(19) United States
(12) Patent Application Publication
(10) Pub. No.: US 2026/0297081 A1
(43) Pub. Date: Oct. 1, 2026

(54) COMPOUNDS AND METHODS OF TREATING CANCERS

(71) Applicants: Cullgen Inc., San Diego, CA (US); Cullgen (Shanghai), Inc., Shanghai (CN)

(72) Inventors: Jing LIU, Oradell, NJ (US); Michael Bruno PLEWE, San Diego, CA (US); Xiaoran HAN, Shanghai (CN); Chengwei ZHANG, Shanghai (CN); Ting YANG, Shanghai (CN); Jing ZHOU, Shanghai (CN); Jie DING, Shanghai (CN)

(21) Appl. No.: 19/162,225

(22) PCT Filed: Mar. 7, 2024
(86) PCT No.: PCT/CN2024/080573

§ 371 Date: Sep. 4, 2025

(30) Foreign Application Priority Data
Mar. 7, 2023 (WO) PCT/CN2023/080134
Dec. 25, 2023 (WO) PCT/CN2023/141582

(51) Int. Cl. C07D 417/12; A61K 31/4439; A61K 31/444; A61K 31/501; A61K 31/519; A61P 35/00; C07D 401/12; C07D 401/14; C07D 471/04; C07D 471/10; C07D 487/20; C07D 491/107

(52) U.S. Cl. CPC C07D 417/12; A61K 31/4439; A61K 31/444; A61K 31/501; A61K 31/519; A61P 35/00; C07D 401/12; C07D 401/14; C07D 471/04; C07D 471/10; C07D 487/20; C07D 491/107

(57) ABSTRACT

Provided herein are heterobifunctional compounds of any of Formulae (I)-(XII), as well as pharmaceutically acceptable salts, pharmaceutical compositions, and the uses thereof, which may be useful for the targeted degradation of proteins of interest. Also provided are E3 ligase binding compounds of Formula (XIII), as well as salts, compositions, and the uses thereof.

### CROSS-REFERENCE

This application claims the benefit of PCT Application No. PCT/CN2023/080134, filed Mar. 7, 2023, PCT Application No. PCT/CN2023/141582, filed Dec. 25, 2023, each of which application is incorporated herein by reference in its entirety.

### SEQUENCE LISTING

The instant application contains a Sequence Listing which has been submitted electronically in XML file format and is hereby incorporated by reference in its entirety. Said XML copy is entitled 54922_723_603_SL.xml, was created on Mar. 1, 2024 and is 6,844 bytes in size.

### FIELD OF THE INVENTION

Provided herein are heterobifunctional compounds and methods useful for the modulation of cyclin D (CCND), cyclin dependent kinase 4 (CDK4), and/or cyclin dependent kinase 6 (CDK6), via ubiquitination and/or targeted protein degradation. Also provided are pharmaceutically acceptable salts of such compounds, pharmaceutical compositions comprising such compounds and salts, and the uses thereof.

The compounds, salts and compositions described herein may be useful for the treatment of various diseases associated with CCND, CDK4 and/or CDK6, including abnormal cell proliferative diseases such as cancer.

### BACKGROUND OF THE INVENTION

The ubiquitin (Ub) proteasome system (UPS) plays a major role in the turnover of intracellular proteins and the maintenance of protein homeostasis, via the selective elimination of abnormally folded or damaged proteins. Targeted protein degradation, which exploits the UPS to selectively degrade a disease-associated protein of interest (“POI”), has emerged as a promising strategy for drug discovery. (M. Békés D. R. Langley and C. M. Crews, PROTAC targeted protein degraders: the past is prologue, Nature Reviews (2022), 21:181-200.)

Targeted protein degraders are heterobifunctional compounds comprising a protein binding moiety (“PBM”) directed to a target protein, which is covalently attached to an E3 ligase binding moiety via a linker. By binding to a target protein and the corresponding E3 ligase, such heterobifunctional degrader molecules may induce ubiquitination and degradation of a POI by the UPS. Typically, the POI is the target protein to which the PBM is bound. Examples of heterobifunctional compounds that degrade a protein which is not directly bound to the PBM have been recently reported. Sometimes termed “bridged” or “bystander” degradation, such off-target degradation may occur unpredictably when an associated protein is ubiquitinated and degraded as part of a degrader-POI complex. Harnessing this phenomenon to selectively target an associated protein of interest could enable the degradation of otherwise undruggable proteins that are associated with a degrader-target protein complex.

Specificity in the UPS is largely achieved through the function of E3 ubiquitin ligases (E3s), which promote the covalent transfer of Ub onto a lysine residue of the substrate protein in a highly regulated manner, leading to polyubiquitination and protein degradation via the 26S proteasome. More than 600 E3s have been identified, which are classified into four families: HECT (homologous to E6AP C terminus) type, U-box type, RING (really interesting new gene) type, and RBR (RING-between-RING) type. (G. Kleiger & T. Mayor, Perilous Journey: a Tour of the Ubiquitin Proteasome System. Trends Cell Biol. (2014), 24:352-359.) However, only a few E3s have been widely used to make degraders, with cereblon (CRBN) and von Hippel-Lindau tumor suppressor (VHL) being the most common. (A. Bricelj et al., E3 Ligase Ligands in Successful PROTACs: An Overview of Syntheses and Attachment Points, Front. Chem. (2021) 9:1-46.) The discovery of degraders acting through different E3 ligases may be useful to address resistance or overcome potential liabilities of CRBN- and VHL-based degraders. (K. Moreau et al., Proteolysis-targeting chimeras in drug development: A safety perspective, Br J Pharmacol (2019), 177:1709-1718.) DDB1 (damaged DNA binding protein 1) was first identified as a subunit of the heterodimeric complex involved in DNA repair. Later, it was discovered that DDB1 functions as a linker protein to connect substrate receptor proteins to CUL4 to assemble multiple CUL4-RING E3 ligase complexes (CRL4). The CRL family of E3 ligases is frequently hijacked by various viruses to degrade different host restriction factors, likely due to the intrinsic flexibility of the CRL ligases. Notably, DDB1 is among the most frequently hijacked E3 factors. Structural analyses of DDB1 in complex with HBx or SV5-V H-Box motifs provided insight into the binding site of DDB1 with viral proteins.

Cyclin-dependent kinases (CDKs) and related serine/threonine protein kinases are important cellular enzymes that perform essential functions in regulating cell division and proliferation. CDKs 1-4, 6, 10 and 11 have been reported to play a direct role in cell cycle progression, while CDKs 3, 5 and 7-9 may play an indirect role (e.g., through activation of other CDKs, regulation of transcription or neuronal functions). The CDK catalytic units are activated by binding to regulatory subunits, known as cyclins, which can be divided into four general classes (G₁, G₁/S, S and M cyclins) whose expression levels vary at different points in the cell cycle. Cyclin B/CDK1, cyclin A/CDK2, cyclin E/CDK2, cyclin D/CDK4, cyclin D/CDK6, and likely other cyclin/CDK heterodimers are important regulators of cell cycle progression.

CDK4 and CDK6 have proven to be tractable targets for development of traditional small molecule inhibitors. Three CDK4/6 inhibitors (palbociclib, ribociclib, and abemaciclib) have been approved for use in combination with endocrine therapy in the treatment of hormone receptor (HR)-positive, human epidermal growth factor receptor 2 (HER2)-negative advanced or metastatic breast cancer, and one (trilaciclib) has been approved to reduce chemotherapy-induced bone marrow suppression. As with other kinase inhibitors, resistance to CDK4/6 inhibitors develops over time, leading to disease progression. Identified mechanisms of resistance to CDK4/6 inhibitors loss of RB1, amplification or overexpression of CDK4 or CDK6, and amplification or overexpression of cyclin E1 (CCNE1) or cyclin E2 (CCNE2). (M.

Alvarez-Fernandez & M. Malumbres, Mechanisms of Sensitivity and Resistance to CDK4/6 Inhibition, Cancer Cell (2020), 37:514-529.) Degraders of CDK4 and CDK6 have been reported, although none appear to have been advanced into clinical development. (see, e.g., B. Jiang et al., Development of Dual and Selective Degraders of Cyclin-Dependent Kinases 4 and 6, Angew Chem Int Ed Engl (2019), 58(19):6321-6326; B. Zhao & K. Burgess, PROTACs suppression of CDK4/6, crucial kinases for cell cycle regulation in cancer, Chem Commun (Camb). (2019), 55(18):2704-2707; S. Rana et al., Selective degradation of CDK6 by a palbociclib based PROTAC, Bioorg Med Chem Lett (2019), 29:1375-1379.)

Cyclin D (CCND) includes three closely related isoforms, cyclin D1 (CCND1), cyclin D2 (CCND2) and cyclin D3 (CCND3), which heterodimerize with CDK4 and CDK6 to form CCND-CDK4/6 complexes that can phosphorylate and inactivate retinoblastoma (Rb). The D-cyclins are activated by mitogenic and oncogenic signals and promote G1-to-S transition, leading to cell proliferation. Amplification or overexpression CCND1 has been observed in solid tumors and hematological cancers, including in breast cancer, bone cancer, central nervous system (CNS) cancer, non-small cell lung cancer (NSCLC), ovarian cancer, endometrial cancer, gastric cancer, oral cancer, esophageal cancer, head and neck cancer, colorectal cancer, pancreatic cancer, melanoma, mantle cell lymphoma (MCL) and multiple myeloma. (S. Chen & L. Li, Degradation strategy of CCND1 in cancer cells and potential clinical application, Front. Oncol. (2022), 1-10.)

The D-cyclins have long been identified as potential therapeutic targets but have proven intractable to traditional small molecule drug discovery efforts due to the lack of actionable binding sites on the protein. (E. A. Musgrove et al., Cyclin D as a therapeutic target in cancer, Nature (2011), 11:558-572.) Cyclin D1 was identified as a top priority cancer target by the Cancer Dependency Map (DepMap) project. (F. M. Behan et al., Prioritization of cancer therapeutic targets using CRISPR-Cas9 screens. Nature (2019), 568:511-516.)

Limited examples of CDK4-targeted degraders that can modulate CCND have been recently reported. (International Publication No. WO 2020/247537; International Publication No. WO 2021/239117; Y. Xiong et al., Bridged Proteolysis Targeting Chimera (PROTAC) Enables Degradation of Undruggable Targets, J. Am. Chem. Soc. (2022), 144:22622-22632.)

Targeted protein degraders may permit the more flexible regulation of protein levels in vitro and in vivo as compared with other techniques, such as gene knockout or short hairpin RNA-mediated (shRNA) knockdown. Unlike gene knockout or shRNA knockdown, a small molecule approach further provides an opportunity to study dose and time dependency in a disease model through modulating the administration routes, concentrations, and frequencies of administration of the corresponding heterobifunctional small molecule compounds.

There remains a need to discover protein degraders that can modulate the activity of CCND, CDK4 and/or CDK6, particularly degraders utilizing novel E3 ligases. Such degraders may be useful for the treatment of disorders associated with CCND, CDK4 and/or CDK6 and may offer advantages in overcoming resistance or reducing toxicity associated with CDK4/6 inhibitors, or with cereblon or VHL-based degraders.

### BRIEF SUMMARY OF THE INVENTION

Described herein are monofunctional and heterobifunctional compounds comprising a DDB1 (damaged DNA binding protein 1) E3 ligase binding moiety. Monofunctional compounds may be useful as molecular glues or as synthetic intermediates for the preparation of heterobifunctional compounds. Heterobifunctional compounds comprising a DDB1 binding moiety conjugated to a protein binding moiety via a linker may be useful for several purposes, including but not limited to use as: 1) antiviral drugs; 2) DDB1 protein level modulators (e.g., increasing or decreasing DDB1 protein levels); 3) DDB1 function modulators (e.g., DDB1 activators or inhibitors); 4) molecular glues (e.g., increasing a protein-protein interaction between DDB1 and a second protein); or 5) targeted protein degraders. The molecular glue or targeted protein degradation functions may be useful for affecting activity or protein levels of the protein bound directly to the PBM (i.e., the target protein) and/or an associated protein of interest (POI) not directly bound to the PBM.

Provided herein are heterobifunctional compounds of any of Formulae (I)-(XII), and embodiments thereof as further described herein, and pharmaceutically acceptable salts, pharmaceutical compositions, and uses thereof.

In one aspect, provided is a heterobifunctional compound of Formula (I):

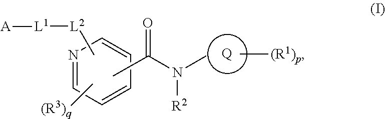

or a pharmaceutically acceptable salt thereof, wherein:
A is a protein binding moiety (PBM) capable of binding to CCND, CDK4 and/or CDK6;
L¹ is a bond or a bivalent linker;
L² is a bond, —NH—, —N(C₁-C₃ alkyl)-, —NHC(O)—, —N(C₁-C₃ alkyl)C(O)—, —C(O)NH—, —C(O)N(C₁-C₃ alkyl)-, —O—, —C₁-C₄-alkylene-, —C₂-C₄-alkenylene- or —C₂-C₄-alkynylene-;
Q is C₆-C₁₀ aryl, 5-10 membered heteroaryl, C₃-C₁₀ cycloalkyl or 4-10 membered heterocyclyl;
each R¹ is independently R^{1A}, R^{1B}, R^{1C}, R^{1D} or R^{1E};
each R^{1A} is independently H, C₁-C₆ alkyl, C₃-C₆ cycloalkyl, or C₁-C₆ heteroalkyl, where each said C₁-C₆ alkyl, C₃-C₆ cycloalkyl, or C₁-C₆ heteroalkyl is optionally substituted by one or more R^{4A};
each R^{1B}, RIG, R^{1D} and R^{1E} is independently H, C₁-C₆ alkyl, C₁-C₆ heteroalkyl, C₁-C₆ alkoxy, OH, D, halo, CN, NO₂, NR^{5A}R^{5B}, C(═O)R^{5A}, C(═O)OR^{5A}, OC(═O)R^{5A}, C(O)NR^{5A}R^{5B}, N(R^{5A})C(O)R^{5B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl, or C₁-C₆ alkoxy is optionally substituted by one or more R^{4B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{4C};
R² is H or C₁-C₃ alkyl;
each R³ is independently R^{3A}, R^{3B} or R^{3C}; or two R³ may be taken together with the atoms to which they are attached to form a C₃-C₁₀ cycloalkyl, 4-10 membered heterocyclyl, C₆-C₁₀ aryl or 5-10 membered heteroaryl, where each said C₃-C₁₀ cycloalkyl, 4-10 membered heterocyclyl, C₆-C₁₀ aryl or 5-10 membered heteroaryl is optionally substituted by one or more R^{6A};
each R^{3A}, R^{3B} and R^{3C} is independently C₁-C₆ alkyl, C₁-C₆ heteroalkyl, C₁-C₆ alkoxy, OH, D, halo, CN, NO₂, NR^{7A}R^{7B}, C(═O)R^{7A}, C(═O)OR^{7A}, OC(═O)R^{7A}, C(O)NR^{7A}R^{7B}, N(R^{7A})C(O)R^{7B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{6B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{6C};
each R^{4A}, R^{4B} and R^{6B} is independently C₁-C₆ alkoxy, oxo, OH, D, halo, CN or NR^{5A}R^{5B};
each R^{4C}, R^{6A} and R^{6C} is independently C₁-C₄ alkyl, C₁-C₆ alkoxy, oxo, OH, D, halo, CN or NR^{9A}R^{9B};
each R^{5A}, R^{5B}, R^{7A}, R^{7B}, R^{8A}, R^{8B}, R^{9A} and R^{9B} is independently H, C₁-C₆ alkyl, C₁-C₆ heteroalkyl, C₃-C₁₀ cycloalkyl, 4-10 membered heterocyclyl, C₆-C₁₀ aryl or 5-10 membered heteroaryl, where each said C₁-C₆ alkyl or C₁-C₆ heteroalkyl is optionally substituted by one or more R^{10A}, and each said C₃-C₁₀ cycloalkyl, 4-10 membered heterocyclyl, C₆-C₁₀ aryl or 5-10 membered heteroaryl is optionally substituted by one or more R^{10B}; or
R^{5A} and R^{5B}, R^{7A} and R^{7B}, R^{8A} and R^{8B}, or R^{9A} and R^{9B} may be taken together with the N-atom to which they are attached to form a 4-10 membered heterocyclyl ring or 5-10 membered heteroaryl ring, where each said 4-10 membered heterocyclyl or 5-10 membered heteroaryl is optionally substituted by one or more R^{10C};
each R^{10A} is independently C₁-C₆ alkoxy, oxo, OH, D, halo, CN or NR^{11A}R^{11B}; each R^{10B} and R^{10C} is independently C₁-C₄ alkyl, C₁-C₆ alkoxy, oxo, OH, D, halo, CN or NR^{12A}R^{12B}.
each R^{11A}, R^{11B}, R^{12A} and R^{12B} is independently H, C₁-C₄ alkyl, or C₃-C₆ cycloalkyl;
p is an integer from 0 to 4; and
q is an integer from 0 to 3;
with the proviso the compound of Formula (I) is not:
2-(3-(2-(2-(2-(4-(6-((6-acetyl-8-cyclopentyl-5-methyl-7-oxo-7,8-dihydropyrido[2,3-d]pyrimidin-2-yl)amino)pyridine-3-yl)piperazin-1-yl)acetamido)ethoxy)ethoxy)propanamido)-N-(4,5-dimethylthiazol-2-yl)-6-methylnicotinamide; or
2-((7-(2-(4-(6-((6-acetyl-8-cyclopentyl-5-methyl-7-oxo-7,8-dihydropyrido[2,3-d]pyrimidin-2-yl)amino)pyridine-3-yl)piperazin-1-yl)acetamido)heptyl)amino)-N-(4,5-dimethylthiazol-2-yl)-6-methylnicotinamide.

In one embodiment, the heterobifunctional compound of Formula (I) has the structure of Formula (II):

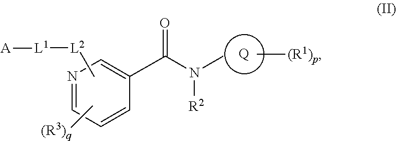

or a pharmaceutically acceptable salt thereof.

In another embodiment, the heterobifunctional compound of Formula (I) or (II) has the structure of Formula (III):

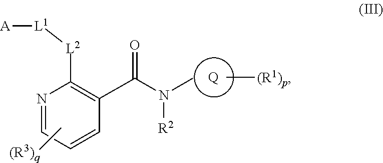

or a pharmaceutically acceptable salt thereof.

In another embodiment, the heterobifunctional compound of Formula (I), (II) or (III) has the structure of Formula (IV):

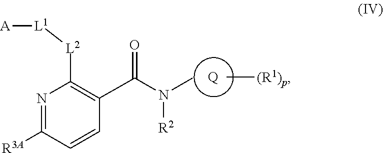

or a pharmaceutically acceptable salt thereof.

In other embodiments, provided herein are heterobifunctional compounds of any of sub-formulae Formulae (V)-(XII), or pharmaceutically acceptable salts thereof, as further described herein.

In another aspect, also provided herein are DDB1 binding compounds of Formula (XIII), as further described, and salts, compositions, and uses thereof.

In another aspect, also provided are pharmaceutical compositions and medicaments comprising the heterobifunctional compounds or salts of any of Formulae (I)-(XII), alone or in combination with additional therapeutic agents, such as additional anticancer therapeutic agents.

In a further aspect, also provided are methods for preparing the heterobifunctional compounds, salts and compositions described, and methods of using the foregoing, for example in methods to degrade a protein of interest (POI), such as CCND or CDK4, or in methods for the treatment of a disease or disorder associated with abnormal cell growth, such as cancer.

All publications, patents, and patent applications mentioned in this specification are herein incorporated by reference in their entirety for the specific purposes identified herein.

### BRIEF DESCRIPTION OF THE DRAWINGS

FIG. 1A shows immunoblots of CCND1, CCND3 and p-Rb proteins expressed by Calu-1 cells after treatment with a dose range of CDK4/6 inhibitor palbociclib or heterobifunctional compound CPD-10 for 4 hours.

FIG. 1B shows immunoblots of CCND1, CCND3, CDK4, FoxM1, Cyclin A2 and p-Rb proteins expressed by MCF7 cells under serum starvation condition after treatment with a dose range of CDK4/6 inhibitor palbociclib or heterobifunctional compound CPD-10 for 24 hours.

FIG. 2A shows immunoblots of CCND1, CCND3, CDK4, p-Rb, FoxM1 and cyclin A2 proteins expressed by T47D cells after treatment with a dose range of heterobifunctional compound CPD-10, or its negative control compound CPD-143 for 48 hours.

FIG. 2B shows anti-viability curves of Calu-1, MCF7 or T47D cells in the presence of CPD-10, or CPD-143 for 3-6 days.

FIG. 3A shows flow cytometric analysis of Annexin V/7-AAD stained T47D cells after treatment with DMSO, palbociclib, or heterobifunctional compound CPD-10 at indicated concentrations for 6 days.

FIG. 3B shows flow cytometric analysis of Annexin V/7-AAD stained HCC1428 cells after treatment with DMSO, palbociclib, heterobifunctional compound CPD-10, or its control compound CPD-143 at indicated concentrations for 6 days.

FIG. 4 shows anti-viability curves of T47D parental or palbo-resistant cells in the presence of palbociclib, or heterobifunctional compounds CPD-10 or CPD-39 for 6 days.

FIG. 5 shows graphs of plasma concentrations of CPD-39 over time after dosing via intravenous (i.v.) injection or oral (p.o.) gavage.

### DETAILED DESCRIPTION

Provided herein are heterobifunctional compounds of any of Formulae (I)-(XII), and pharmaceutically acceptable salts, pharmaceutical compositions, and uses thereof. Also provided are monofunctional compounds of Formula (XIII), and salts, compositions, and uses thereof. Various embodiments of the foregoing, including enumerated embodiments E1 to E85, are described herein.

Features specified in each embodiment may be combined with other specified features to provide further embodiments.

The present invention may be understood more readily by reference to the following detailed description of the aspects and embodiments of the invention, as well as the Examples described herein.

### Definitions

As used herein and in the appended claims, the singular forms “a”, “and”, and “the” include plural referents unless the context clearly dictates otherwise. Thus, for example, reference to “an agent” includes a plurality of such agents, and reference to “the cell” includes reference to one or more cells (or to a plurality of cells), and so forth unless otherwise indicated.

As used herein, “and/or” means “one or more” of a list of alternatives, unless otherwise indicated. For example, references to “CCND, CDK4 and/or CDK6” means “one or more of CCND, CDK4 or CDK6.”

When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between ±1% and ±15% of the stated number or numerical range, and preferably within ±10%.

The invention described herein may be suitably practiced in the absence of any element(s) not specifically disclosed herein. Thus, for example, in each instance herein any of the terms “comprising”, “consisting essentially of”, and “consisting of” may be replaced with either of the other two terms. As used in the specification and appended claims, unless specified to the contrary, the following terms have the meaning indicated below.

“Amino” refers to an “unsubstituted amino” radical of the form —NH₂ or a “substituted amino” radical of the form —NHR or —N(R)₂ as described herein and defined by the claims.

“Azido” or “azide” refers to the —N₃ radical.

“Cyano” refers to the —CN radical.

“Halo” or “halogen” refers to bromo, chloro, fluoro or iodo substituents.

“Hydrazino” refers to the ═N—NH₂ radical.

“Hydroxy” refers to the —OH radical.

“Imino” refers to the ═N—H radical.

“Nitro” refers to the —NO₂ radical.

“Oxa” refers to the —O— radical.

“Oximo” refers to the ═N—OH radical.

“Oxo” refers to the ═O radical.

“Thioxo” refers to the ═S radical.

“Alkyl” refers to a straight or branched hydrocarbon chain radical consisting solely of carbon and hydrogen atoms, containing no unsaturation, having the specified number of carbon atoms. In certain embodiments, an alkyl comprises from one to fifteen carbon atoms (e.g., C₁-C₁₅ alkyl). In certain embodiments, an alkyl comprises one to thirteen carbon atoms (e.g., C₁-C₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), I-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

Unless stated otherwise, an alkyl group (including an alkenyl or alkynyl group) may be optionally substituted by one or more substituent groups, as further defined by the claims and disclosure herein. The total number of substituent groups may equal the total number of hydrogen atoms on the alkyl moiety, to the extent such substitution makes chemical sense. Substituted alkyl groups typically contain from 1 to 6 optional substituents, sometimes 1 to 5 optional substituents, 1 to 4 optional substituents, or preferably 1 to 3 optional substituents. Optional substituent groups are independently selected unless otherwise stated.

“Alkenyl” refers to an alkyl group, as defined herein, consisting of at least two carbon atoms and at least one carbon-carbon double bond. In certain embodiments, an alkenyl comprises from two to fifteen carbon atoms (e.g., C₂-C₁₅ alkenyl). In certain embodiments, an alkenyl comprises two to thirteen carbon atoms (e.g., C₂-C₁₃ alkenyl). In certain embodiments, an alkenyl comprises two to eight carbon atoms (e.g., C₂-C₈ alkenyl). In other embodiments, an alkenyl comprises two to five carbon atoms (e.g., C₂-C₈ alkenyl). In other embodiments, an alkenyl comprises two to four carbon atoms (e.g., C₂-C₄ alkenyl). In other embodiments, an alkenyl comprises two to three carbon atoms (e.g., C₂-C₃ alkenyl). Unless stated otherwise, alkenyl groups may be optionally substituted by one or more substituent groups, as further defined by the claims and disclosure herein.

“Alkynyl” refers to an alkyl group, as defined herein, consisting of at least two carbon atoms and at least one carbon-carbon triple bond. In certain embodiments, an alkynyl comprises from two to fifteen carbon atoms (e.g., C₂-C₁₅ alkynyl). In certain embodiments, an alkynyl comprises two to thirteen carbon atoms (e.g., C₂-C₁₃ alkynyl). In certain embodiments, an alkynyl comprises two to eight carbon atoms (e.g., C₂-C₈ alkynyl). In other embodiments, an alkynyl comprises two to five carbon atoms (e.g., C₂-C₈ alkynyl).

In other embodiments, an alkynyl comprises two to four carbon atoms (e.g., C₂-C₄ alkynyl). In other embodiments, an alkynyl comprises two to three carbon atoms (e.g., C₂-C₃ alkynyl). Unless stated otherwise, alkynyl groups may be optionally substituted by one or more substituent groups, as further defined by the claims and disclosure herein.

Exemplary groups suitable as optional substituents on an alkyl, alkenyl or alkynyl moiety include, but are not limited to: halo (preferably F), cyano, nitro, oxo, thioxo, imino, oximo, trimethylsilanyl, R^{a}, —OR^{a}, —SR, —OC(O)R^{a}, —N(R)₂, —C(O)R, —C(O)OR, —C(O)N(R^{a})₂, —N(R)C(O)OR^{a}, —OC(O)—N(R^{a})₂, —N(R^{a})C(O)R^{a}, —OC(O)—OR^{a}, —O—R^{c}—C(O)N(R^{a})₂, —N(R^{a})S(O)ₜR^{a}, —S(O)ₜOR^{a}, —S(O)ₜR^{a} and —S(O)ₜN(R^{a})₂ (each where t is 1 or 2), where each R^{a} is independently hydrogen, alkyl, fluoroalkyl, cycloalkyl, cycloalkylalkyl, carbocyclyl, carbocyclylalkyl, heterocyclyl, heterocyclylalkyl, aryl, arylalkyl, heteroaryl, or heteroarylalkyl, and R^{c} is a straight or branched alkylene or alkenylene chain, and where each of the above substituents is unsubstituted or optionally further substituted, as indicated, with groups suitable for the substituent type. For example, alkyl or alkylene moieties (including the “alkyl” portion of cycloalkylalkyl, carbocyclylalkyl or heterocyclylalkyl groups, and the like) may be optionally further substituted with F, oxo, OH, alkoxy, amino, alkylamino or dialkylamino; cycloalkyl or heterocyclyl moieties (including the “cyclic” portion of cycloalkylalkyl, carbocyclylalkyl, heterocyclylalkyl groups) may be optionally further substituted with alkyl, F, oxo, OH, alkoxy, amino, alkylamino or dialkylamino; and aryl or heteroaryl moieties (including the “aromatic” portion of arylalkyl and heteroarylalkyl groups) may be optionally further substituted with halo, OH, alkoxy, CN, amino, alkylamino or dialkylamino.

“Alkylene” refers to a straight or branched bivalent hydrocarbyl group having the specified number of carbon atoms, which can link two other groups together. Sometimes it refers to a straight chain group —(CH₂)ₜ— where t is 1-8, and preferably t is 1-4. Examples include, e.g., methylene, ethylene, propylene, n-butylene, and the like. Typically, an alkylene chain has from one to twelve carbon atoms (C₁-C₁₂ alkylene), sometimes one to eight carbon atoms (e.g., C₁-C₈ alkylene), one to five carbon atoms (e.g., C₁-C₅ alkylene), one to four carbon atoms (e.g., C₁-C₄ alkylene), one to three carbon atoms (e.g., C₁-C₃ alkylene), one to two carbon atoms (e.g., C₁-C₂ alkylene), or one carbon atom (e.g., C₁ alkylene). In other embodiments, an alkylene comprises five to eight carbon atoms (e.g., C₅-C₅ alkylene), two to five carbon atoms (e.g., C₂-C₈ alkylene), or three to five carbon atoms (e.g., C₃-C₅ alkylene). Where specified, an alkylene can also be substituted by other groups and may include one or more degrees of unsaturation (i.e., an alkenylene or alkynylene chain) or rings. The open valences of an alkylene need not be at opposite ends of the chain. Also included within the scope of the term ‘alkylenes’ are branched alkylene groups, such as —CH(Me)-, —CH₂CH(Me)- and —C(Me)₂—, or cyclic groups such as cyclopropan-1,1-diyl and unsaturated groups such as ethylene (—CH═CH—) or propylene (—CH₂—CH═CH—). Where an alkylene group is described as optionally substituted, the substituents may include those typically present on alkyl groups as described herein and defined by the claims.

“Alkoxy” refers to a radical bonded through an oxygen atom of the formula —O-alkyl, where alkyl is an alkyl chain as defined above. Similarly, “thioalkoxy” refers to a radical bonded through a sulfur atom of the formula —S-alkyl, where alkyl is an alkyl chain as defined above.

In some instances, substituted alkyl groups are specifically named by reference to the substituent group. For example, “haloalkyl” refers to an alkyl group of the indicated number of carbon atoms that is substituted by one or more halogens. Examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. “Fluoroalkyl” refers to an alkyl radical specifically substituted by one or more fluoro radicals, e.g., trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally further substituted as defined above for an alkyl group.

An “alkoxyalkyl” refers to an alkyl group substituted by one or more alkoxy substituents, e.g., methoxymethyl (—CH₂OMe) or 2-ethoxyethyl (—CH₂CH₂OEt).

An “aminoalkyl” refers to an alkyl group substituted by one or more substituted or unsubstituted amino substituents, e.g., aminomethyl (—CH₂NH₂), aminoethyl (—CH₂CH₂NH₂), NN-dimethylaminoethyl (—CH₂CH₂N(Me)₂) or N-pyrrolidinylethyl (—CH₂CH₂—N-pyrrolidinyl).

A “hydroxyalkyl” refers to an alkyl group substituted by one or more hydroxy substituents, e.g., hydroxymethyl (—CH₂OH) or 2-hydroxyethyl (—CH₂CH₂OH).

“Heteroalkyl”, “heteroalkenyl” and “heteroalkynyl” refer to substituted or unsubstituted alkyl, alkenyl or alkynyl groups, in which one or more skeletal chain atoms is replaced by a heteroatom selected from O, N, S, P, or Si, or combinations thereof, wherein nitrogen, sulfur and phosphorus heteroatoms may optionally be oxidized, and nitrogen heteroatoms may optionally be substituted or quaternized. Heteroalkyl groups include the indicated number of chain atoms and one or more heteroatoms selected from —O—, —N(R″)—, —S(O)— and —S(O)₂—, where R″ is H or C₁-C₄ alkyl unless otherwise indicated. If given, a numerical range refers to the chain length in total, including both carbon and chain heteroatoms. For example, a 2-8 membered heteroalkyl has a chain length of 2 to 8 atoms, including both carbon and chain heteroatoms. Such a heteroalkyl chain may be referred to herein as a “C₂-C₈ heteroalkyl”. Connection to the rest of the molecule may be through either a heteroatom or a carbon in the heteroalkyl, heteroalkenyl or heteroalkynyl chain. Unless stated otherwise as unsubstituted, a heteroalkyl, heteroalkenyl, or heteroalkynyl group may be optionally substituted by one or more substituents such as those described herein as suitable for alkyl moieties. Bivalent heteroalkyl, heteroalkenyl and heteroalkynyl moieties may be referred to respectively as heteroalkylene, heteroalkenylene or heteroalkynylene moieties of the indicated chain length. It will be understood that the number and location of heteroatoms in a saturated or unsaturated heteroalkyl chain is limited to extent that such compounds are chemically stable (i.e., excluding peroxide moieties, disulfide moieties, and the like).

“Aryl” or “aromatic” refers to a radical derived from an aromatic monocyclic or multicyclic hydrocarbon ring system by removing a hydrogen atom from a ring carbon atom. Bivalent aryl moieties may be referred to as arylene moieties. The aromatic monocyclic or multicyclic hydrocarbon ring system contains only hydrogen and carbon. Typically aryl groups contain six to twenty carbon atoms (“C₆-C₂₀” aryl), six to fourteen carbon atoms (“C₆-C₁₄” aryl), six to twelve carbon atoms (“C₆-C₁₂” aryl), or preferably six to ten carbon atoms (“C₆-C₁₀” aryl) as ring members, where at least one of the rings in the ring system is fully unsaturated, i.e., it contains a cyclic, delocalized (4n+2) p-electron system in accordance with the Hückel theory. The ring system from which aryl groups are derived include, but are not limited to, groups such as benzene, fluorene, indane, indene, tetralin, and naphthalene. Unless stated otherwise specifically in the specification, the term “aryl” or the prefix “ar-” (such as in “arylalkyl” or “aralkyl”) is meant to include aryl radicals optionally substituted by one or more substituents as defined in the claims and as further described below.

“Arylalkyl” or “aralkyl” refers to a radical of the formula —R^{c}-aryl where Re is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group. The number of carbon atoms in the alkyl and aryl portions of the arylalkyl moiety, respectively, may be indicated together or separately. For example, a benzyl group may be described as C₇-arylalkyl or in the alternative as C₆-aryl-C₁-alkyl.

“Carbocyclyl” or “cycloalkyl” refers to a stable non-aromatic monocyclic or polycyclic hydrocarbon radical consisting solely of carbon and hydrogen atoms, which may be monocyclic, or include fused, spirocyclic or bridged ring systems, having from three to fifteen carbon atoms (e.g., a “C₃-C₁₅ cycloalkyl”). Such a cycloalkyl ring systems may be referred to in the alternative as a 3-15 membered cycloalkyl. In some embodiments, a cycloalkyl ring comprises three to thirteen carbon atoms (e.g., a “C₃-C₁₃ cycloalkyl”). In some embodiments, a cycloalkyl ring comprises three to twelve carbon atoms (e.g., a “C₃-C₁₂ cycloalkyl”). In some embodiments, a cycloalkyl ring comprises three to ten carbon atoms (e.g., a “C₃-C₁₀ cycloalkyl”). In other embodiments, a cycloalkyl may comprise three to eight carbon atoms (e.g., a “C₃-C₈ cycloalkyl”) or five to seven carbon atoms (e.g., a “C₅-C₇ cycloalkyl”) or three to six carbon atoms (e.g., a “C₃-C₆ cycloalkyl”). The cycloalkyl may be attached to the rest of the molecule by a single bond or an exocyclic double bond. A carbocyclyl may be fully saturated (i.e., containing single C—C bonds only) or partially unsaturated (i.e., containing one or more double bonds or triple bonds). A fully saturated carbocyclyl radical is also referred to as “cycloalkyl.” Partially unsaturated carbocyclyl rings may also be referred to as cycloalkenyl or cycloalkynyl moieties. Bivalent cycloalkyl moieties may be referred to as cycloalkylene moieties. Unless stated otherwise specifically in the specification, the terms “carbocyclyl” and “cycloalkyl” are meant to include carbocyclyl radicals that are optionally substituted by one or more substituents as defined in the claims and as further described below.

Examples of monocyclic cycloalkyls include, e.g., cyclopropyl, cyclobutyl, cyclopentyl, cyclohexyl, cycloheptyl, and cyclooctyl. Examples of monocyclic cycloalkenyls include, e.g., cyclopentenyl, cyclohexenyl, cycloheptenyl, or cyclooctenyl. Examples of bridged cycloalkyls include, e.g., adamantyl (i.e., tricyclo[3.3.1.1]decanyl), norbomyl (i.e., bicyclo[2.2.1]heptanyl), norbornenyl (i.e., bicyclo[2.2.1]hept-2-enyl), or 7,7-dimethyl-bicyclo[2.2.1]heptanyl. Examples of fused cycloalkyls include, e.g., decalinyl, bicyclo[4.3.0]nonanyl, bicyclo[3.3.0]octanyl. Examples of spirocyclic cycloalkyls include, e.g., spiro[3.3]heptanyl, spiro[3.4]octanyl, spiro[4.4]nonyl, spiro[4.5]decanyl, or spiro[5.5]undecanyl.

“Carbocyclylalkyl” or “cycloalkylalkyl” refers to a radical of the formula —R^{c}-carbocyclyl where Re is an alkylene chain as defined above. The alkylene chain and the carbocyclyl radical are optionally substituted as defined above.

“Heterocyclyl” or “heterocycloalkyl” refers to a stable 3- to 20-membered non-aromatic ring radical that comprises two to fourteen carbon atoms and from one to six heteroatoms (preferably from one to three heteroatoms) selected from nitrogen, oxygen, and sulfur (i.e., N, O and S(O)_{z}, where z is 0, 1 or 2). Such a ring system may be monocyclic, or include fused, spirocyclic or bridged ring systems. In some embodiments, the heterocyclyl ring system comprises 3-20 ring atoms (including both carbon and heteroatom ring atoms), referred to herein as a 3-20 membered heterocyclyl. In some embodiments, the heterocyclyl ring system comprises 3-14 ring atoms, i.e., is a 3-14 membered heterocyclyl. In some embodiments, the heterocyclyl ring system is a 3-12 membered heterocyclyl, preferably containing from 1-3 heteroatoms as described above. In some embodiments, the heterocyclyl ring system is a 3-10 membered heterocyclyl, preferably containing from 1-3 heteroatoms as described above. In some embodiments, the heterocyclyl ring system is a 3-8 membered heterocyclyl, preferably containing from 1-3 heteroatoms as described above. In some embodiments herein, the heterocyclyl ring system comprises a 5-6 membered heterocyclyl, a 3-8 membered heterocyclyl, a 4-13 membered heterocyclyl, or a 4-10 membered heterocyclyl, wherein each such heterocyclyl preferably contains from 1-3 heteroatoms. Bivalent heterocycloalkyl moieties may be referred to as “heterocyclene” moieties. It will be understood that the number and location of heteroatoms in a heterocyclic ring is limited to extent that such compounds are chemically stable. The heteroatoms in the heterocyclyl radical are optionally oxidized. One or more nitrogen atoms, if present, are optionally quaternized. The heterocyclyl radical is partially or fully saturated. The heterocyclyl may be attached to the rest of the molecule through a C or N atom of the ring(s). Unless stated otherwise specifically in the specification, the term “heterocyclyl” is meant to include heterocyclyl radicals that are optionally substituted by one or more substituents as defined in the claims and as further described below.

Examples of heterocyclyl radicals include, e.g., azetidinyl, pyrrolidinyl, piperidinyl, piperazinyl, azepanyl, diazepanyl, tetrahydrofuranyl, tetrahydropyranyl, tetrahydrothiophenyl, tetrahydrothiopyranyl, morpholinyl, thiomorpholinyl, dioxolanyl, thienyl[1,3]dithianyl, decahydroisoquinolyl, imidazolinyl, imidazolidinyl, isothiazolidinyl, isoxazolidinyl, octahydroindolyl, octahydroisoindolyl, 2-oxopiperazinyl, 2-oxopiperidinyl, 2-oxopyrrolidinyl, 4-piperidonyl, quinuclidinyl, trithianyl, pyrazolidinyl, oxazolidinyl, thiazolidinyl, 1-oxo-thiomorpholinyl, and 1,1-dioxo-thiomorpholinyl. Spirocyclic heterocyclyl radicals include, e.g., 2-azaspiro[3.3]heptanyl, 2,6-diazaspiro[3.3]heptanyl, 2-azaspiro[3.4]octanyl, 2,6-diaza-spiro[3.4]octanyl, 2-azaspiro[4.4]nonanyl, 2,7-diazaspiro[4.4]nonanyl, 2-azaspiro[4.5]decanyl, 2,8-diaza-spiro[4.5]decanyl, 3-azaspiro[5.5]undecanyl, or 3,9-diazaspiro[5.5]undecanyl.

Cycloalkyl and heterocyclyl moieties described herein as optionally substituted may be substituted by one or more substituent groups, which are selected independently unless otherwise indicated. The total number of substituent groups may equal the total number of hydrogen atoms on the cycloalkyl or heterocyclyl moiety, to the extent such substitution makes chemical sense. Optionally substituted cycloalkyl or heterocyclyl groups typically contain from 1 to 5 optional substituents, sometimes 1 to 4 optional substituents, preferably 1 to 3 optional substituents, or more preferably from 1 to 2 optional substituents.

Exemplary groups suitable as substituents on a cycloalkyl or heterocyclyl moiety include: alkyl, fluoroalkyl alkenyl, alkynyl, halo (preferably F), cyano, nitro, oxo, thioxo, imino, oximo, trimethylsilanyl, R^{a}, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—N(R^{a})C(O)OR^{a}, —R^{b}—OC(O)N(R^{a})₂, —R_{b}—N(R^{a})C(O)R^{a}, —R^{b}—OC(O)—OR^{a}, —R^{b}—O—R^{c}—C(O)N(R^{a})₂, —R^{b}—N(R^{a})S(O)ₜR^{a}, —R^{b}—S(O)ₜOR^{a}, —R^{b}—S(O)ₜR^{a} and —R^{b}—S(O)ₜN(R^{a})₂ (each where t is 1 or 2), where each R^{a} is independently hydrogen, alkyl, fluoroalkyl, cycloalkyl, cycloalkylalkyl, carbocyclyl, carbocyclylalkyl, heterocyclyl, heterocyclylalkyl, aryl, arylalkyl, heteroaryl, or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated, or optionally further substituted as indicated with groups suitable for the substituent type.

“N-heterocyclyl” or “N-linked heterocyclyl” refers to a heterocyclyl radical as defined above containing at least one nitrogen and where the point of attachment of the heterocyclyl radical to the rest of the molecule is through a nitrogen atom in the heterocyclyl radical. An N-linked heterocyclyl radical is optionally substituted as described above for heterocyclyl radicals. Examples of such N-linked heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-linked heterocyclyl” refers to a heterocyclyl radical as defined above containing at least one heteroatom and where the point of attachment of the heterocyclyl radical to the rest of the molecule is through a carbon atom in the heterocyclyl radical. A C-linked heterocyclyl radical is optionally substituted as described above for heterocyclyl radicals. Examples of such C-linked heterocyclyl radicals include, but are not limited to, 2-morpholinyl, 2- or 3- or 4-piperidinyl, 2-piperazinyl, 2- or 3-pyrrolidinyl, and the like.

“Heteroaryl” or “heteroaromatic” refers to a radical derived from a monocyclic, fused bicyclic or polycyclic hydrocarbon ring system by removing a hydrogen atom from a ring atom, where at least one ring carbon atom has been replaced by N, O, or S, and wherein at least one of the rings in the ring system is fully unsaturated, i.e., it contains a cyclic, delocalized (4n+2) p-electron system in accordance with the Hückel theory. The inclusion of heteroatoms permits aromaticity in 5-membered rings as well as 6-membered rings. Typically, heteroaryl groups contain 5 to 20 ring atoms (“5-20 membered heteroaryl”), sometimes 5 to 14 ring atoms (“5-14 membered heteroaryl”), or 5 to 12 ring atoms (“5-12 membered heteroaryl”), and preferably 5 to 10 ring atoms (“5-10 membered heteroaryl”), in each case including both carbon and hetero- ring atoms. Heteroaryl rings are attached to the base molecule via a ring atom of the heteroaromatic ring, such that aromaticity is maintained. Thus, 6-membered heteroaryl rings may be attached to the base molecule via a ring C atom, while 5-membered heteroaryl rings may be attached to the base molecule via a ring C or N atom. Bivalent heteroaryl moieties may be referred to as heteroarylene moieties. Unless stated otherwise specifically in the specification, the term “heteroaryl” or the prefix “heteroar-” (such as in “heteroaralkyl”) is meant to include heteroaryl radicals optionally substituted by one or more substituents as defined in the claims and as further described below. By analogy to arylalkyl moieties, the terms “heteroarylalkyl” or “heteroaralkyl” refers to a radical of the formula —R^{c}-heteroaryl.

Examples of heteroaryl rings include, but are not limited to, pyrrole, furan, thiophene, pyrazole, imidazole, isoxazole, oxazole, isothiazole, thiazole, triazole (including 1,2,3-triazole and 1,3,4-triazole), oxadiazole (including 1,2,3-oxadiazole, 1,2,4-oxadiazole, 1,2,5-oxadiazole, and 1,3,4-oxadiazole), thiadiazole (including 1,2,3-thiadiazole, 1,2,4-thiadiazole, 1,2,5-thiadiazole, and 1,3,4-thiadiazole), tetrazole, pyridine, pyridazine, pyrimidine, pyrazine, triazine, benzofuran, benzothiophene, indole, benzimidazole, indazole, benzotriazole, pyrrolopyridine (including pyrrolo[2,3-b]pyridine, pyrrolo[2,3-c]pyridine, pyrrolo[3,2-b]pyridine, pyrrolo[3,2-c]pyridine), imidazopyridine (including imidazo[4,5-b]pyridine, imidazo[4,5-c]pyridine), pyrazolopyridine, (including pyrazolo[4,3-d]pyridine, pyrazolo[4,3-c]pyridine, pyrazolo[3,4-c]pyridine, pyrazolo[3,4-b]pyridine), isoindole, indazole, purine, indolizine, imidazopyridine (including imidazo[1,2-a]pyridine, imidazo[1,5-a]pyridine), pyrazolo[1,5-a]pyridine, pyrrolo[1,2-b]pyridazine, imidazo[1,2-c]pyrimidine, quinoline, isoquinoline, cinnoline, quinazoline, quinoxaline, phthalazine, naphthyridine (including 1,6-naphthyridine, 1,7-naphthyridine, 1,8-naphthyridine, 1,5-naphthyridine, 2,6-naphthyridine, 2,7-naphthyridine), pyridopyrimidine (including pyrido[3,2-d]pyrimidine, pyrido[4,3-d]pyrimidine, pyrido[3,4-d]pyrimidine, pyrido[2,3-d]pyrimidine), pyridopyrazine (including pyrido[2,3-b]pyrazine, pyrido[3,4-b]pyrazine), pyrimidopyrimidine (including pyrimido[5,4-d]pyrimidine, pyrimido[4,5-d]pyrimidine), pyrazino[2,3-b]pyrazine, and carbazole. In preferred embodiments, 5- or 6-membered heteroaryl groups are selected from the group consisting of pyrrolyl, furanyl, thiophenyl, pyrazolyl, imidazolyl, isoxazolyl, oxazolyl, isothiazolyl, thiazolyl, triazolyl, oxadiazolyl, thiadiazolyl, pyridinyl, pyrimidinyl, pyrazinyl and pyridazinyl rings.

Aryl and heteroaryl moieties described herein as optionally substituted may be substituted by one or more substituent groups, which are selected independently unless otherwise indicated. The total number of substituent groups may equal the total number of hydrogen atoms on the aryl or heteroaryl moiety, to the extent such substitution makes chemical sense and aromaticity is maintained. Optionally substituted aryl or heteroaryl groups typically contain from 1 to 5 optional substituents, sometimes 1 to 4 optional substituents, preferably 1 to 3 optional substituents, or more preferably from 1 to 2 optional substituents.

Exemplary groups suitable as substituents on an aryl or heteroaryl moiety include: alkyl, fluoroalkyl alkenyl, alkynyl, halo, cyano, nitro, trimethylsilanyl, R^{a}, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—N(R^{a})C(O)OR^{a}, —R^{b}—OC(O)N(R^{a})₂, —R^{b}—N(R^{a})C(O)R^{a}, —R^{b}—OC(O)—OR^{a}, —R^{b}—O—R^{c}—C(O)N(R^{a})₂, —R^{b}—N(R^{a})S(O)R^{a}, —R^{b}—S(O)ₜOR^{a}, —R^{b}—S(O)R^{a} and —R^{b}—S(O)ₜN(R^{a})₂ (each where t is 1 or 2), where each R^{a} is independently hydrogen, alkyl, fluoroalkyl, cycloalkyl, cycloalkylalkyl, carbocyclyl, carbocyclylalkyl, heterocyclyl, heterocyclylalkyl, aryl, arylalkyl, heteroaryl, or heteroarylalkyl, each R^{b} is independently a direct bond or a straight or branched alkylene or alkenylene chain, and R^{C} is a straight or branched alkylene or alkenylene chain, and where each of the above substituents is unsubstituted unless otherwise indicated, or optionally further substituted as indicated with groups suitable for the substituent type.

“N-heteroaryl” or “N-linked heteroaryl” refers to a heteroaryl radical as defined above containing at least one nitrogen and where the point of attachment of the heteroaryl radical to the rest of the molecule is through a N atom in the heteroaryl radical. An N-linked heteroaryl radical is optionally substituted as described above for heteroaryl radicals.

“C-heteroaryl” or “C-linked heteroaryl” refers to a heteroaryl radical as defined above and where the point of attachment of the heteroaryl radical to the rest of the molecule is through a carbon atom in the heteroaryl radical. A C-linked heteroaryl radical is optionally substituted as described above for heteroaryl radicals.

“Optional” or “optionally” means that the subsequently described event or circumstance may, but need not necessarily, occur, and the description includes instances where the event or circumstance occurs and instances in which it does not.

The terms “optionally substituted” and “substituted or unsubstituted” may be used interchangeably to indicate that the group being described may have no non-hydrogen substituents (i.e., is unsubstituted), or the group may have one or more non-hydrogen substituents (i.e., is substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the groups are independently selected and may be the same or different. Throughout the disclosure, it will be understood that the number and nature of optional substituent groups will be limited to the extent that such substitutions make chemical sense to yield chemically stable molecules.

As used herein, the term “reactive functional group” refers to atoms, or associated groups of atoms, that are intended, or may reasonably be expected, to undergo chemical reaction. Examples of reactive functional groups include, but are not limited to: protected or unprotected primary or secondary amine; carboxylic acid, carboxylate ester, or an activated derivative thereof (e.g., acyl halide, acid anhydride, Weinreb amide, activated ester, etc.); halo, hydroxy, formyl, oxo (═O), sulfonate ester (—OSO₂R^{°} where R^{°} is alkyl, haloalkyl or aryl, e.g., mesylate, triflate or tosylate), boronic acid, boronate ester; azide; alkyl moieties substituted by halo, hydroxy, oxo, sulfonate ester, boronic acid, boronate ester, carboxylic acid, carboxylate ester, alkoxy, amide, ketone, etc.; aldehyde, ketone, amide, nitrile, imine, or acetal; aryl or heteroaryl groups substituted by halo, hydroxy, sulfonate ester, boronic acid, or boronate ester; α,β-unsaturated amide, α,β-unsaturated ketone, α,β-unsaturated acid, or α,β-unsaturated ester; α-halo amide, α-halo ketone, α-halo acid, or α-halo ester; sulfonic acid or sulfonyl halide; protected or unprotected thiol, thioether, or disulfide; alkene or alkyne moieties; epoxide, aziridine, or the like. Suitable protecting groups for protected amines include carbamates (e.g., Boc, Cbz, Fmoc or Alloc), silyl protecting groups (e.g., trimethylsilyl (TMS), triethylsilyl (TES), tert-butyldimethylsilyl (TBS), tert-butyldiphenylsilyl (TBDPS), triisopropylsilyl (TIPS)), benzylic protecting groups (e.g., benzyl (Bn) or p-methoxybenzyl (PMB)), and other suitable protecting groups known in the art. P. G. M. Wuts, Greene's Protective Groups in Organic Synthesis, Wiley, 5^{th} Ed.

The compounds disclosed herein, in some embodiments, contain one or more asymmetric centers and may give rise to enantiomers, diastereomers, racemates, or other stereoisomeric forms or mixtures thereof, which in some embodiments are defined in terms of absolute stereochemistry as (R)- or (S)-. Unless otherwise indicated, it is intended that all stereoisomeric forms of the compounds disclosed herein are contemplated by this disclosure. Bonds of compounds containing asymmetric centers may be depicted using a solid line (-), a solid wedge ( [symbol] ), or a dotted wedge ( [symbol] ). The use of a solid line to depict a bond to an asymmetric center is meant to indicate that the stereochemistry is undefined, and all possible stereoisomers (e.g., specific isomers, racemic mixtures, etc.) at the stereocenter are included. The use of a solid or dotted wedge to depict a bond to an asymmetric center is meant to indicate that only the stereoisomer shown is included. Where defined, the absolute configuration may also be indicated by an (R)- or (S)-designation. In compounds containing more than one asymmetric center, depictions with both solid lines and solid or dotted wedges at different asymmetric centers indicate the structure is undefined at the stereocenter depicted using the solid line.

The compounds disclosed herein also include geometric isomers, atropisomers, other conformational isomers and/or tautomeric forms. When the compounds described herein contain alkenyl groups, it is intended that this disclosure includes both E and Z geometric isomers (e.g., cis or trans) of an alkene double bond, unless otherwise indicated. Positional isomers (e.g., structural isomers such as ortho-, meta-, and para-isomers around a benzene ring) may be included if indicated by a variable point of attachment in the structure as drawn.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The ratio of tautomers depends on several factors, including physical state, temperature, solvent, and pH. Examples of tautomeric equilibrium include:

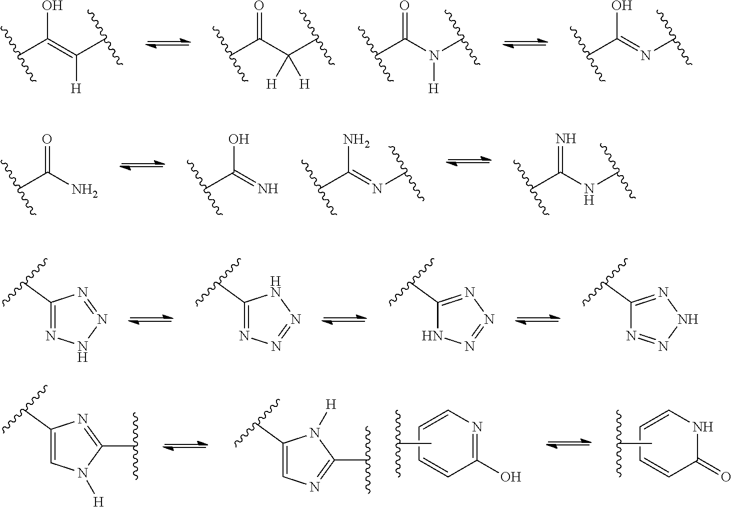

Unless otherwise stated, structures depicted herein are intended to include compounds which differ only in the presence of one or more isotopically enriched atoms, which are otherwise identical to those recited in one of the formulae provided, but for the fact that one or more atoms are replaced by an atom having an atomic mass or mass number different from the atomic mass or mass number usually found in nature. For example, included within the scope of the present disclosure are compounds having the present structures except for the replacement of one or more hydrogen atoms by a deuterium (²H) or tritium (³H), or the replacement of a carbon atom by ¹³C- or ¹⁴C-enriched carbon.

The compounds of the present disclosure optionally contain unnatural proportions of atomic isotopes at one or more atoms that constitute such compounds. For example, the compounds may be labeled with isotopes of hydrogen, carbon, nitrogen, oxygen, fluorine, phosphorus, sulfur, chlorine, bromine, or iodine. Isotopic substitution with ²H, ³H, ¹¹C, ¹³C, ¹⁴C, ¹⁵C, ¹²N, ¹³N, ¹⁵N, ¹⁶N, ¹⁶O, ¹⁷O, ¹⁸O, ¹⁴F, ¹⁵F, ¹⁶F, ¹⁷F, ¹⁸F, ³¹P, ³²P, ³³S, ³⁴S, ³⁵S, ³⁶S, ³⁵Cl, ³⁷Cl, ⁷⁹Br, ⁸¹Br, ¹²⁵I are contemplated. All isotopic variations of the compounds of the present invention, whether radioactive or not, are encompassed within the scope of the present invention. In some embodiments, the compounds herein include isotopic forms enriched in the content of ²H, ³H, ¹C, ¹³C and/or ¹⁴C.

Certain isotopically-labeled compounds of the invention, for example those into which radioactive isotopes such as ¹³H and ¹⁴C are incorporated, may be useful in drug and/or substrate tissue distribution assays. Tritiated, i.e., ³H, and carbon-14, i.e., ¹⁴C, isotopes are particularly preferred for their ease of preparation and detectability.

Substitution with heavier isotopes, such as deuterium, i.e., ²H, may also afford certain therapeutic advantages such as greater metabolic stability, increased in vivo half-life, increased duration of action, or reduced dosage requirements. In some embodiments, a compound is deuterated in at least one position. In certain embodiments, a compound disclosed herein has some or all the H atoms replaced with ²H atoms.

Isotopically-labeled compounds can generally be prepared by carrying out the procedures disclosed in the Schemes and/or in the Examples below, substituting an isotopically-labeled reagent for a non-isotopically-labeled reagent. Methods for the synthesis for deuterium-containing compounds are known and include, by way of non-limiting example only, the procedures described in U.S. Pat. Nos. 5,846,514 and 6,334,997.

Unless indicated otherwise, all references to compounds herein include references to salts (including pharmaceutically acceptable salts), solvates (including hydrates), and complexes thereof, as well as to solvates and complexes of the salts thereof, and isotopically labelled versions thereof.

In certain embodiments, a compound or salt described herein as “substantially pure”, means that it contains less than about 5%, preferably less than about 10%, and or more preferably less than about 0.1% of other organic small molecules, such as unreacted intermediates or synthetic by-products that are created, for example, in one or more of the steps of a synthesis method.

Compounds described herein may exist in the form of salts. The term “salts” refers to inorganic and organic salts of a compound herein. Such salts may be prepared in situ during the isolation and purification of a compound, or by separately treating the compound with a suitable organic or inorganic acid or base and isolating the salt thus formed.

“Pharmaceutically acceptable salts” are salts that retain the biological effectiveness and properties of the free base compound that are suitable for administration to a subject. Reference to “a pharmaceutically acceptable salt” of compounds described herein is intended to encompass any pharmaceutically suitable salt form. Preferred pharmaceutically acceptable salts include pharmaceutically acceptable acid addition salts and pharmaceutically acceptable base addition salts. Non-pharmaceutically acceptable salts, including salts of chiral acids, may also be useful for synthesis, isolation, purification, chiral resolution, and the like.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M et al., “Pharmaceutical Salts,” Journal of Pharmaceutical Science, 66:1-19 (1997)).

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Salts can be prepared according to methods known in the art, for example by mixing a solution of the basic or acidic compound and the desired acid or base, respectively. The resulting salt form may be isolated by precipitation and filtration or may be recovered by evaporation of the solvent. Compounds existing in free base form having a basic functionality maybe converted to the acid addition salts by treating with a stoichiometric excess of the appropriate acid. Such acid addition salts may be reconverted to the corresponding free base by treating with a stoichiometric excess of a suitable base, such as potassium carbonate or sodium hydroxide, typically in the presence of aqueous solvent at a temperature between about 0° C. and 100° C. The free base form may be isolated by conventional means, such as extraction into an organic solvent. Acid addition salts may be interchanged by taking advantage of differential solubilities of the salts, volatilities or acidities of the acids, or by treating with the appropriately loaded ion exchange resin. For example, the interchange may be affected by the reaction of a salt with a slight stoichiometric excess of an acid of a lower pK than the acid component of the starting salt. Such interconversions are typically carried out at a temperature between about 0 [symbol] and the boiling point of the solvent being used as the medium for the procedure. Similar exchanges are possible with base addition salts, typically via the intermediacy of the free base form.

The compounds and salts of any of Formulae (I)-(XIII) may exist in unsolvated or solvated forms. A “solvate” refers to a molecular complex comprising a compound or salt and one or more solvent molecules. Preferably, the solvate includes one or more pharmaceutically acceptable solvents, such as water or ethanol. The term “hydrate” is use when said solvent is water. Hydrates may be classified as isolated site, channel, or metal-ion coordinated hydrates. When solvent or water molecules are tightly bound, the complex may have a well-defined stoichiometry independent of humidity. When solvent or water molecules are weakly bound, the solvent or water content may be non-stoichiometric and depend on humidity or drying conditions.

Also included herein are multi-component complexes (other than salts and solvates) wherein the compound of any of Formulae (I)-(XII) and at least one other component are present in stoichiometric or non-stoichiometric amounts. Examples of such complexes include clathrates (i.e., drug-host inclusion complexes) and co-crystals, which typically are crystalline complexes wherein the constituents are bound together through non-covalent interactions.

A “prodrug” refers to a masked compound that functions as a drug precursor, which may itself have little or no pharmacological activity, and that releases the active drug in vivo following administration via a chemical or physiological process (e.g., due to exposure to physiological pH or through enzymatic action). See, e.g., ‘Pro-drugs as Novel Delivery Systems, Vol. 14, ACS Symposium Series (T. Higuchi and W. Stella); ‘Bioreversible Carriers in Drug Design’, Pergamon Press, 1987 (ed. E. B. Roche, American Pharmaceutical Association); “Design of Prodrugs” by H. Bundgaard (Elsevier, 1985). Compounds or salts of any of Formulae (I)-(XII) may be administered in the form of prodrugs.

### Heterobifunctional Compounds

Provided herein are heterobifunctional compounds of any of Formulae (I)-(XII) and pharmaceutically acceptable salts thereof, and pharmaceutical compositions comprising such compounds and salts. The heterobifunctional compounds described herein comprise a DNA damage-binding protein 1 (DDB1) binding moiety, a bivalent linker, and a protein binding moiety (PBM). In some embodiments, the DDB1 binding moiety is a natural product. In some embodiments, the DDB1 binding moiety is a synthetic product. In some embodiments, the PBM is capable of binding to CCND, CDK4 and/or CDK6. In certain preferred embodiments, the PBM is capable of binding to CDK4 and/or CDK6. Such compounds or salts may be useful for several purposes, including modulating the protein level or activity of CCND, CDK4 and/or CDK6.

Enumerated embodiments E1 to E49 relate to compounds of Formulae (I) to (XII), and pharmaceutically acceptable salts thereof, as well as pharmaceutical compositions of such compounds and salts.

E1. A heterobifunctional compound of Formula (I):

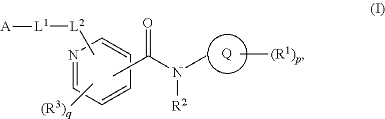

or a pharmaceutically acceptable salt thereof, as described in the Brief Summary of the Invention.
   E2. The heterobifunctional compound of embodiment E1, having the structure of Formula (II):

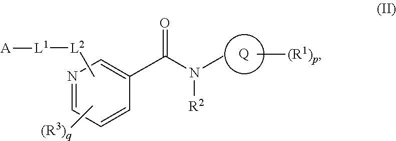

or a pharmaceutically acceptable salt thereof.
   E3. The heterobifunctional compound of embodiment E1 or E2, having the structure of Formula (III):

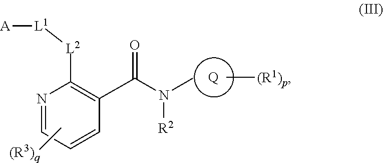

or a pharmaceutically acceptable salt thereof.
   E4. The heterobifunctional compound of any one of embodiments E1 to E3, having the structure of Formula (IV):

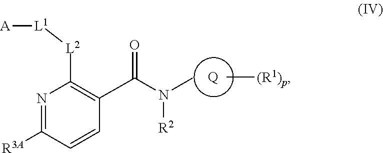

or a pharmaceutically acceptable salt thereof.
   E5. The heterobifunctional compound of any one of embodiments E1 to E4, or a pharmaceutically acceptable salt thereof, wherein Q is selected from the group consisting of:

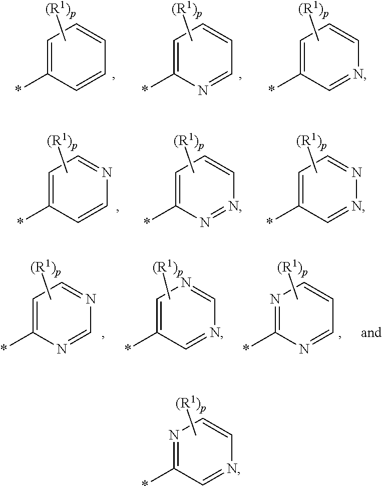

* represents the point of attachment; and
R¹ and p are defined as in Formula (I).
   E6. The heterobifunctional compound of embodiment E5, or a pharmaceutically acceptable salt thereof, wherein Q is selected from the group consisting of:

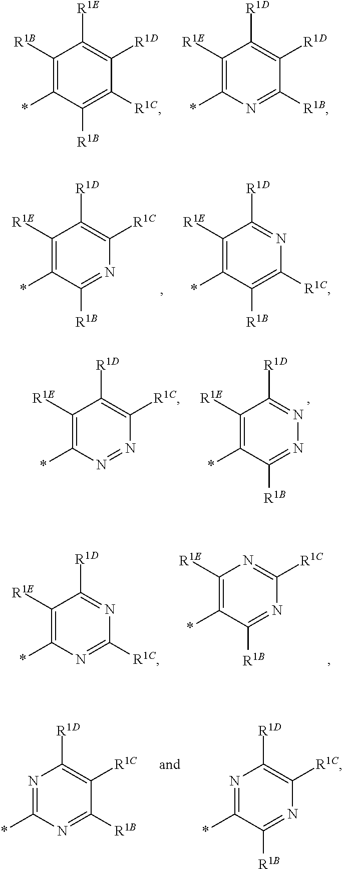

wherein:
* represents the point of attachment; and
R^{1B}, R^{1C}, R^{1D} and R^{1E} are defined as in Formula (I).
   E7. The heterobifunctional compound of any one of embodiments E1 to E4, or a pharmaceutically acceptable salt thereof, wherein Q is selected from the group consisting of:

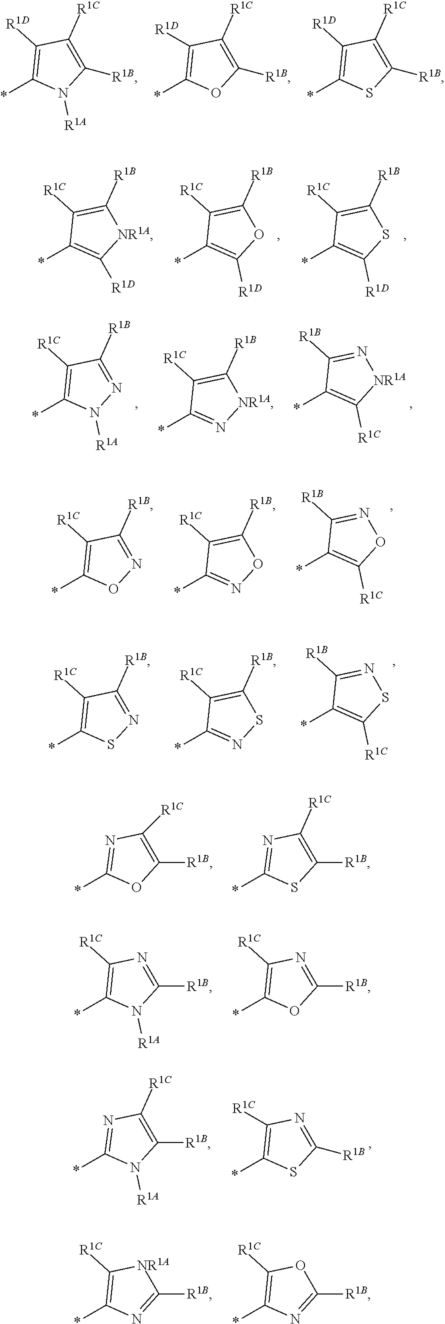

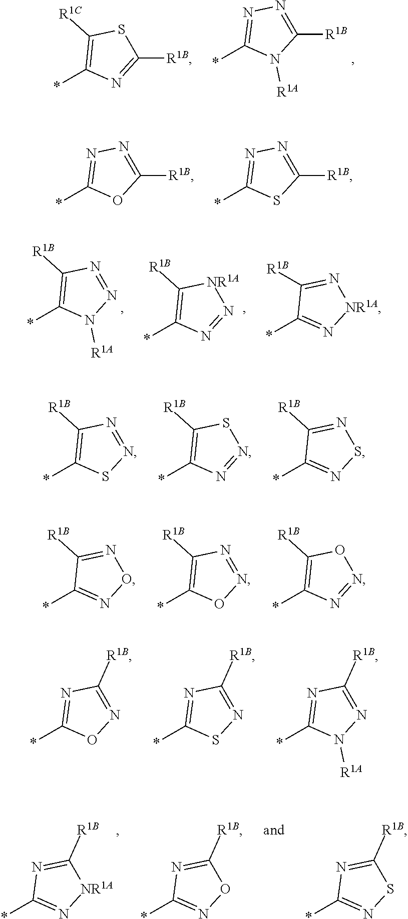

wherein:
* represents the point of attachment; and
R^{1A}, R^{1B}, R^{1C} and R^{1D} are defined as in Formula (I).
   E8. The heterobifunctional compound of embodiment E7, or a pharmaceutically acceptable salt thereof, wherein Q is selected from the group consisting of:

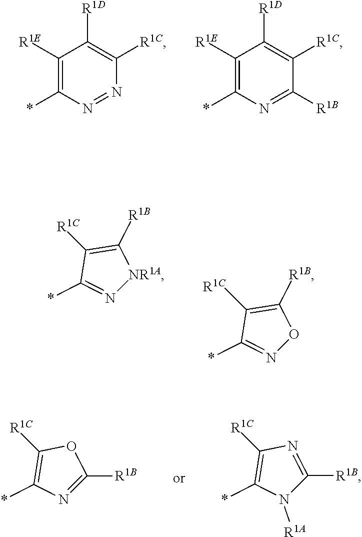

wherein:
* represents the point of attachment; and
R^{1A}, R^{1B}, R^{1C}, R^{1D} and R^{1E} are defined as in Formula (I).
   E9. The heterobifunctional compound of embodiment E3, having the structure of Formula (V):

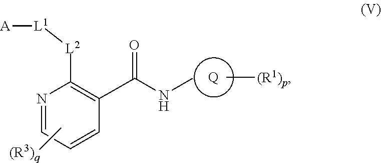

or a pharmaceutically acceptable salt thereof, wherein:
R¹, R³, A, L¹, L², p and q are defined as in Formula (I); and
Q is a C₆ aryl or a 6-membered heteroaryl.
   E10. The heterobifunctional compound of embodiment E9, having the structure of Formula (V-A), (V-B), (V-C) or (V-D):

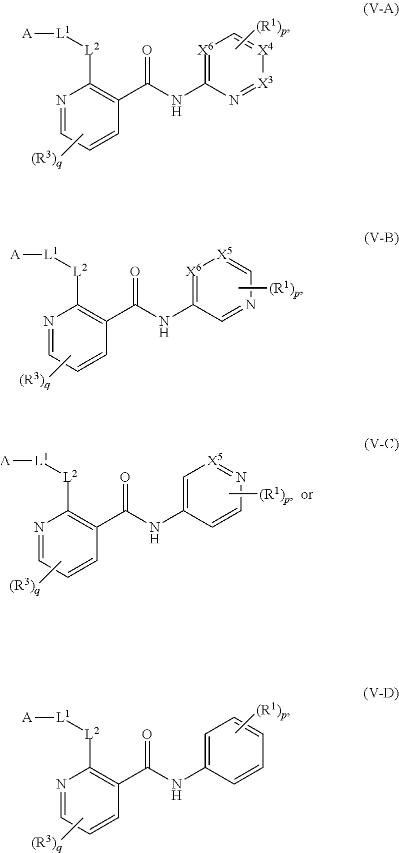

or a pharmaceutically acceptable salt thereof, wherein:
R¹, R², R³, A, L, L², p, q, R^{1B} R^{1C}, R^{1D} and R^{1E} are defined as in Formula (I);
X³ is N or CR^{1B};
X⁴ is N or CR^{1C};
X⁵ is N or CR^{1D}; and
X⁶ is N or CR^{1E}.
   E11. The heterobifunctional compound of embodiment E9 or E10, having the structure:

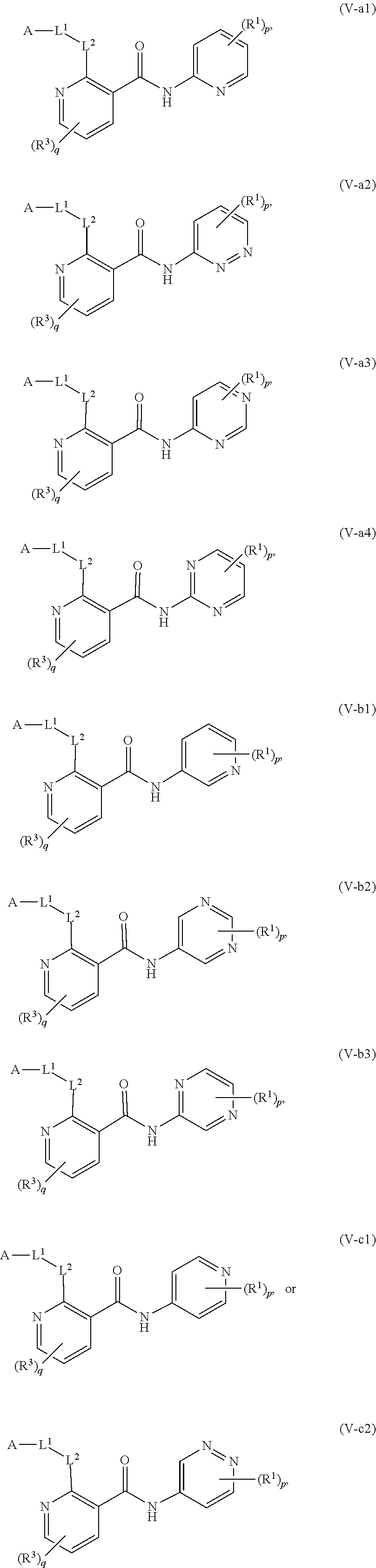

or a pharmaceutically acceptable salt thereof, wherein:
R¹, R², R³, A, L, L¹, p, and q are defined as in Formula (I).
   E12. The heterobifunctional compound of embodiment E3, having the structure of Formula (VI):

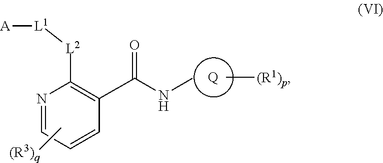

or a pharmaceutically acceptable salt thereof, wherein:
R¹, R³, A, L, L², p and q are defined as in Formula (I); and
Q is a 5-membered heteroaryl.
   E13. The heterobifunctional compound of embodiment E12, having the structure:

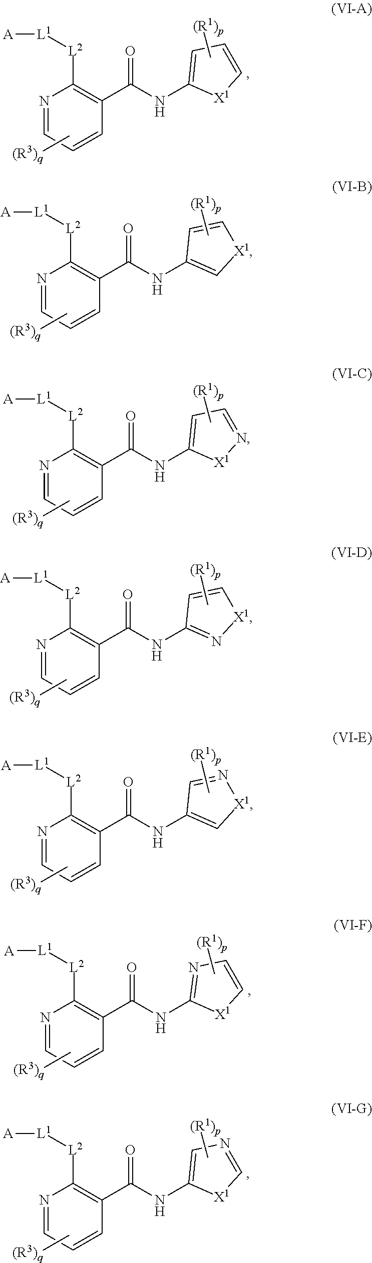

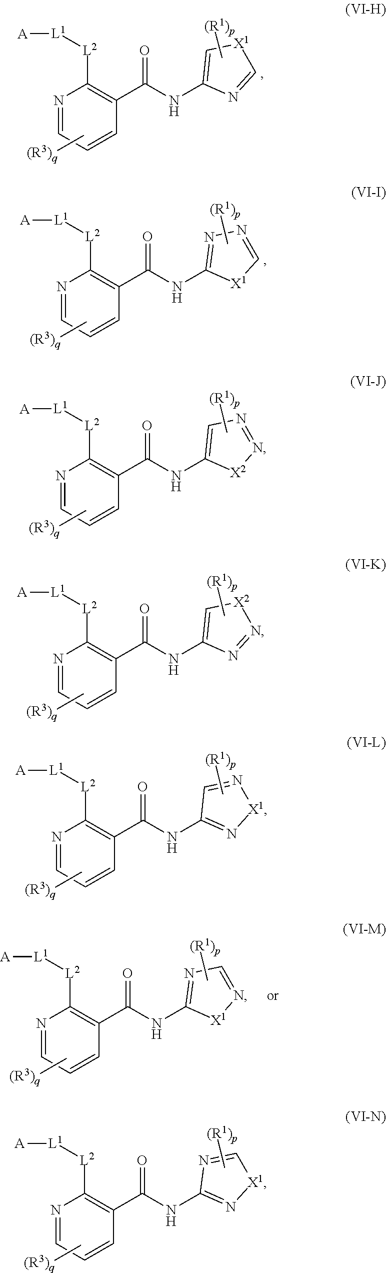

or a pharmaceutically acceptable salt thereof, wherein:
R¹, R³, A, L¹, L², p, q and R^{1A} are defined as in Formula (I);
X¹ is O, S or NR^{1A}; and
X¹ is O, S or NR^{1A}.
   E14. The heterobifunctional compound of embodiment E13, having the structure:

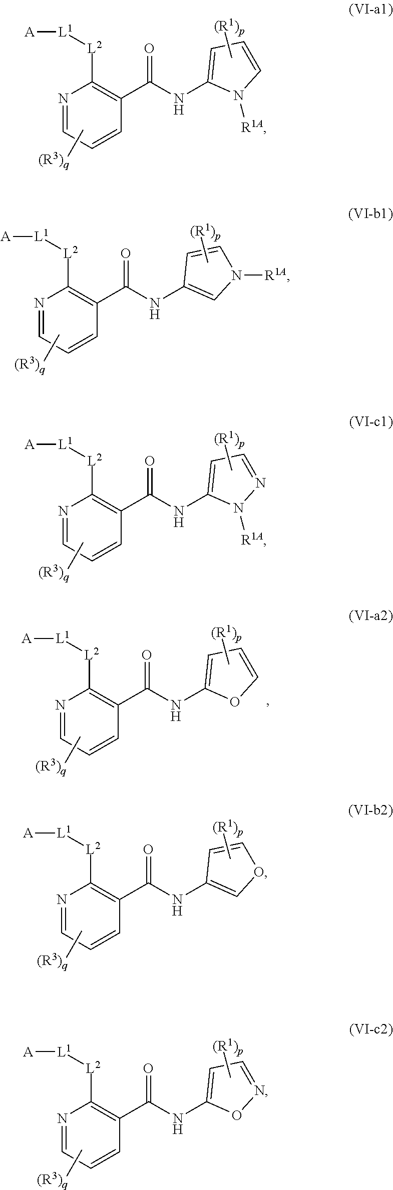

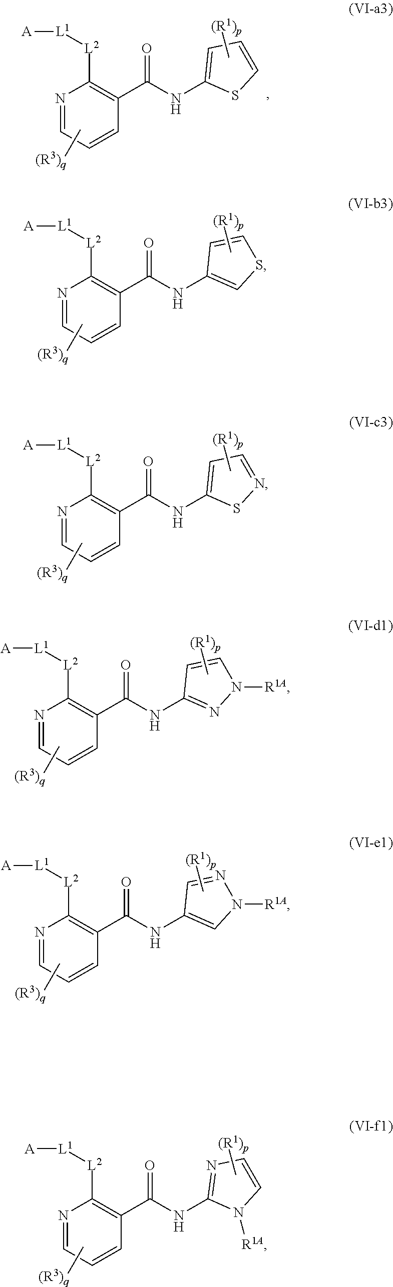

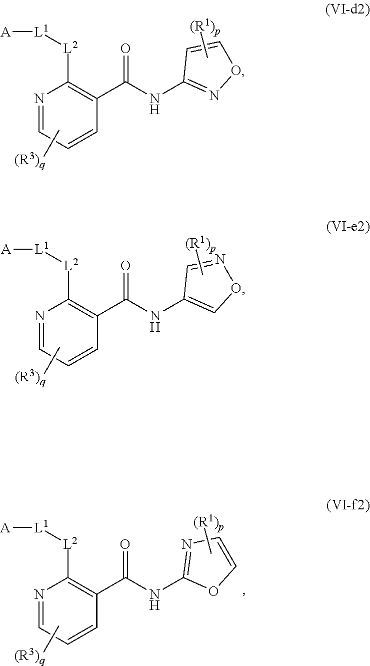

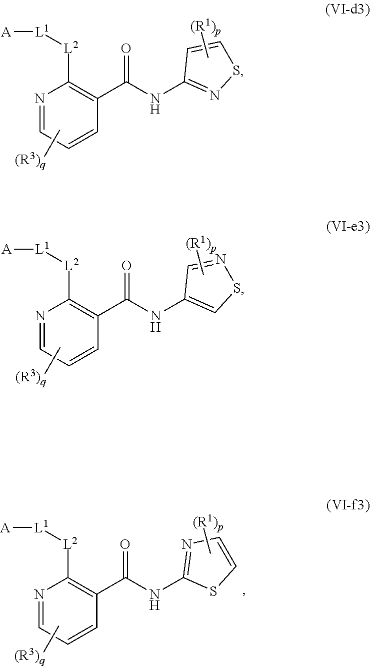

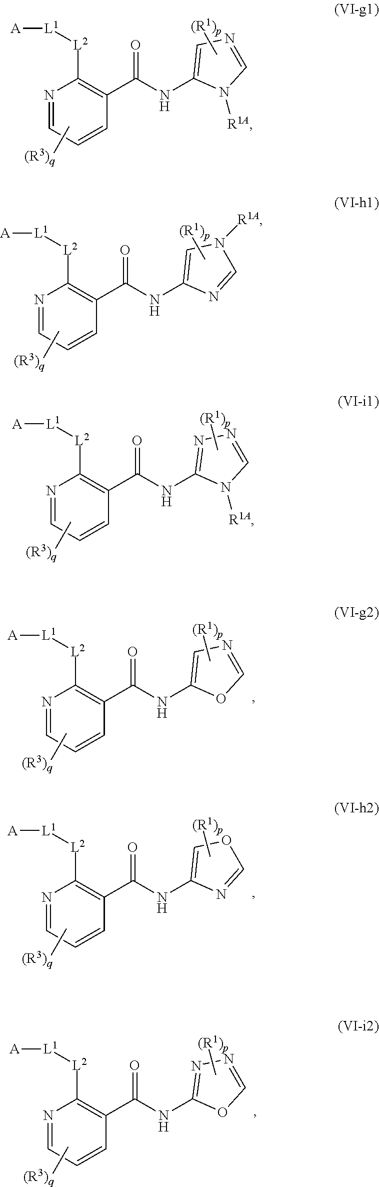

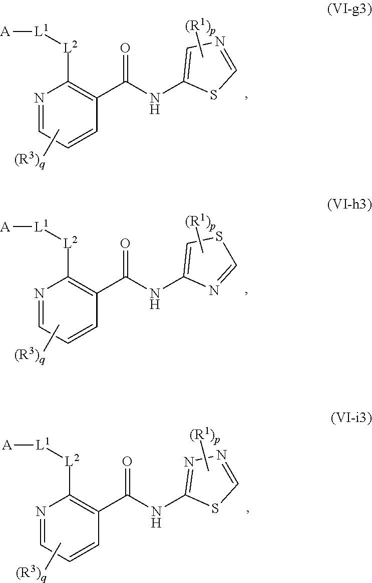

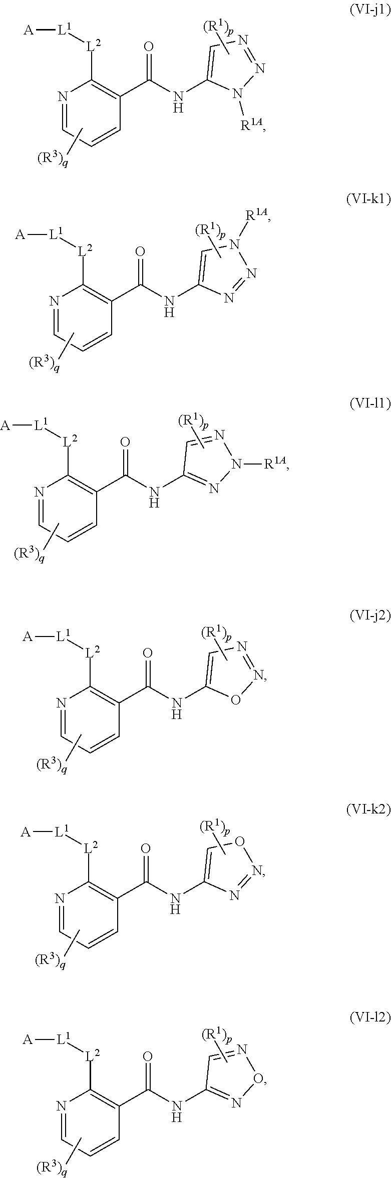

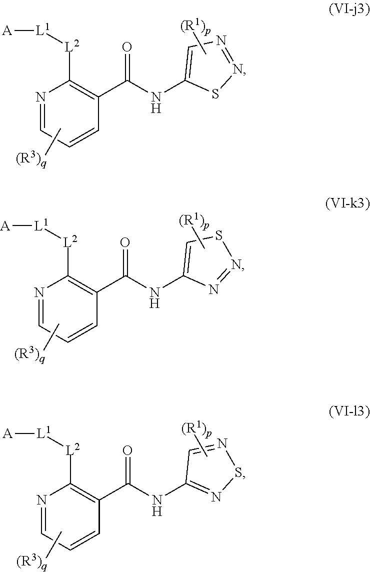

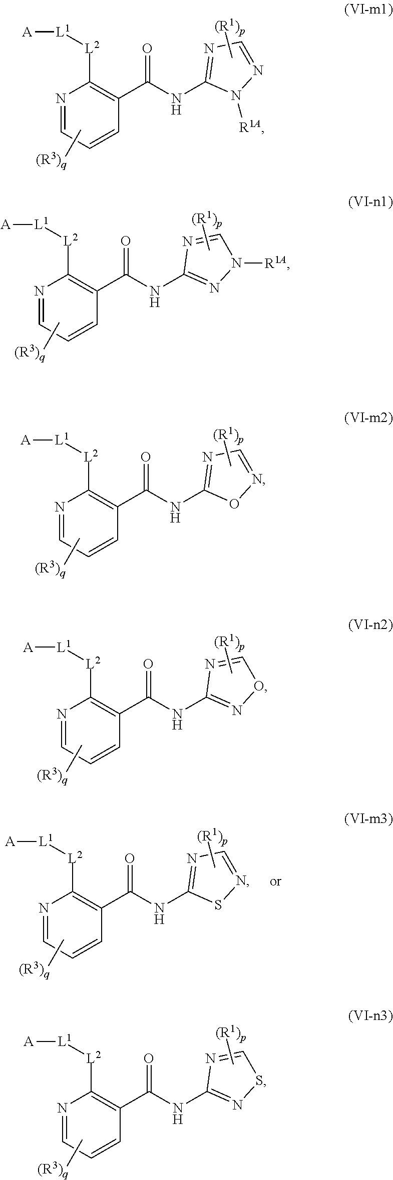

or a pharmaceutically acceptable salt thereof, wherein:
R¹, R³, A, L¹, L², p, q and R^{1A} are defined as in Formula (I).
   E15. The heterobifunctional compound of embodiment E4, having the structure of Formula (VII):

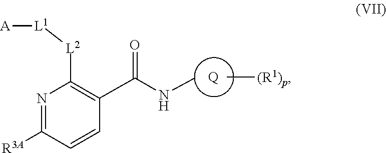

or a pharmaceutically acceptable salt thereof, wherein:
R¹, R^{3A}, A, L¹, L² and p are defined as in Formula (I); and
Q is a C₆ aryl or a 6-membered heteroaryl.
   E16. The heterobifunctional compound of embodiment E15, having the structure of Formula (VII-A), (VII-B), (VII-C) or (VII-D):

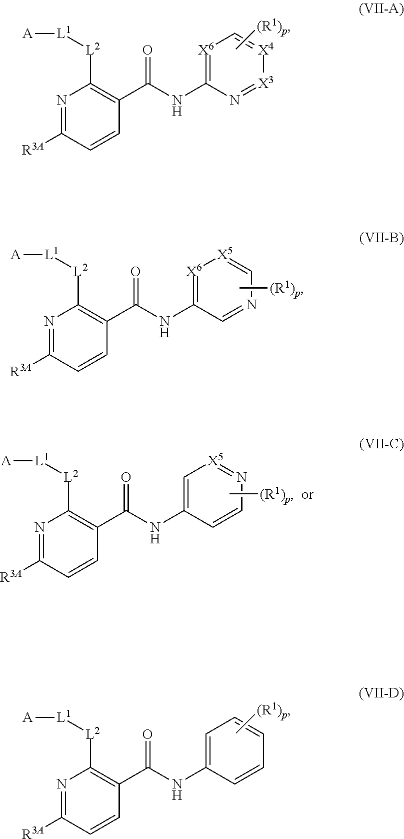

or a pharmaceutically acceptable salt thereof, wherein:
R¹, R^{3A}, A, L¹, L², p, R^{1B}, R^{1C}, R_{1D} and R^{1E} are defined as in Formula (I);
X³ is N or CR^{1B};
X⁴ is N or CR^{1C};
X⁵ is N or CR^{1D}; and
X⁶ is N or CR^{1E}.
   E17. The heterobifunctional compound of embodiment E15 or E16, having the structure:

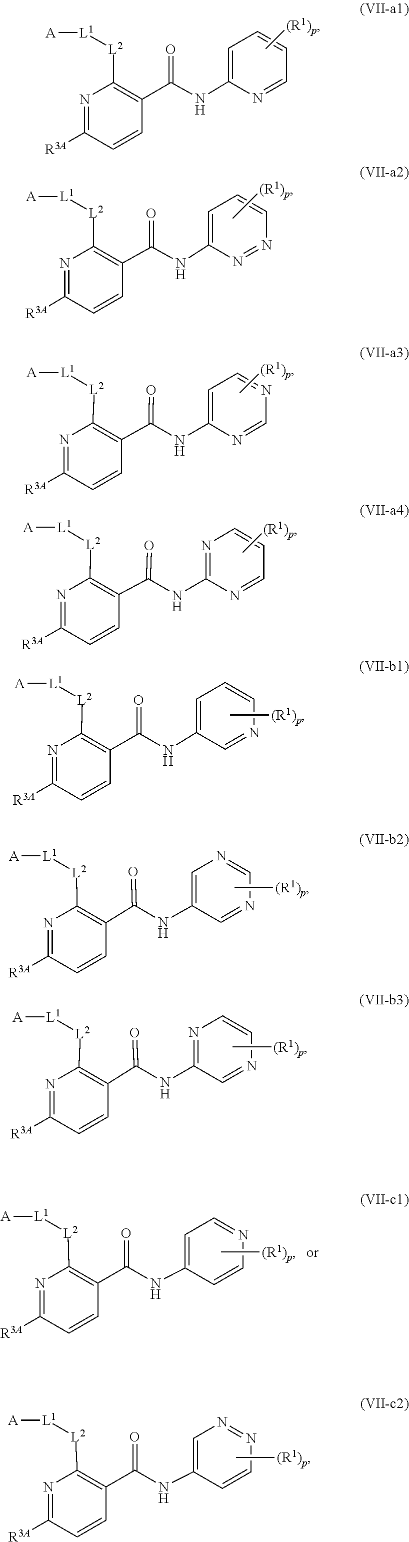

or a pharmaceutically acceptable salt thereof, wherein:
R¹, R^{1A}, A, L, L², and p are defined as in Formula (I).
   E18. The heterobifunctional compound of embodiment E4, E15, E16 or E17, having the structure of Formula (IX) selected from:

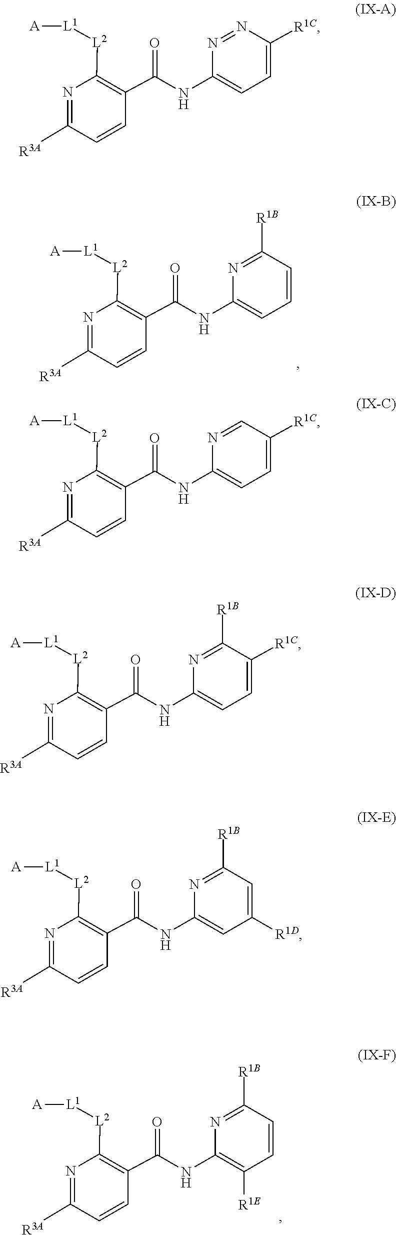

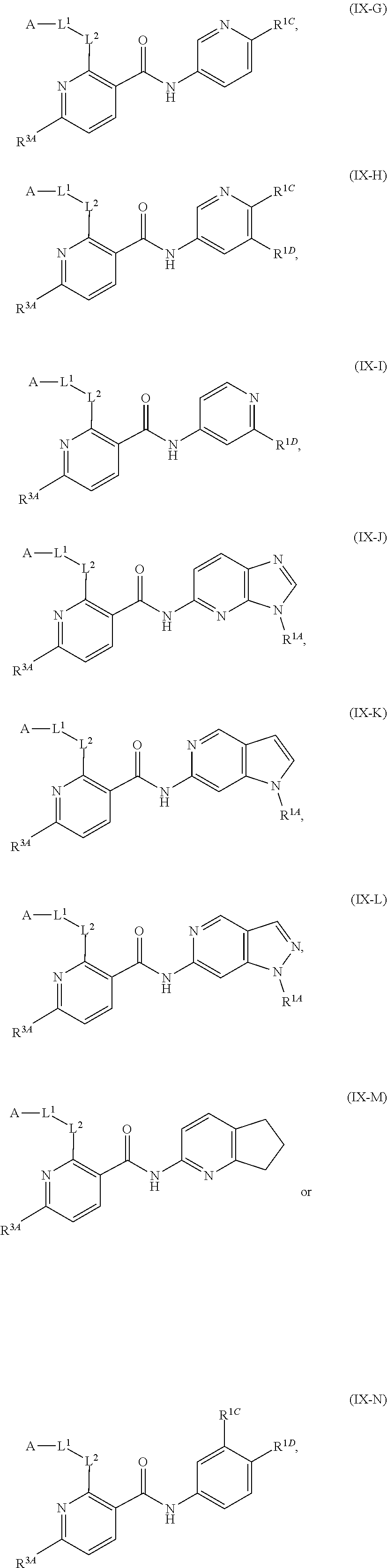

or a pharmaceutically acceptable salt thereof, wherein:
R^{1A}, R^{1B}, R^{1C}, R^{3A}, A, L¹ and L² are defined as in Formula (I).
   E19. The heterobifunctional compound of embodiment E4, having the structure of Formula (VIII):

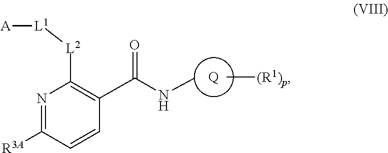

or a pharmaceutically acceptable salt thereof, wherein:
R¹, R^{3A}, A, L¹, L² and p are defined as in Formula (I); and
Q is a 5-membered heteroaryl.
   E20. The heterobifunctional compound of embodiment E19, having the structure:

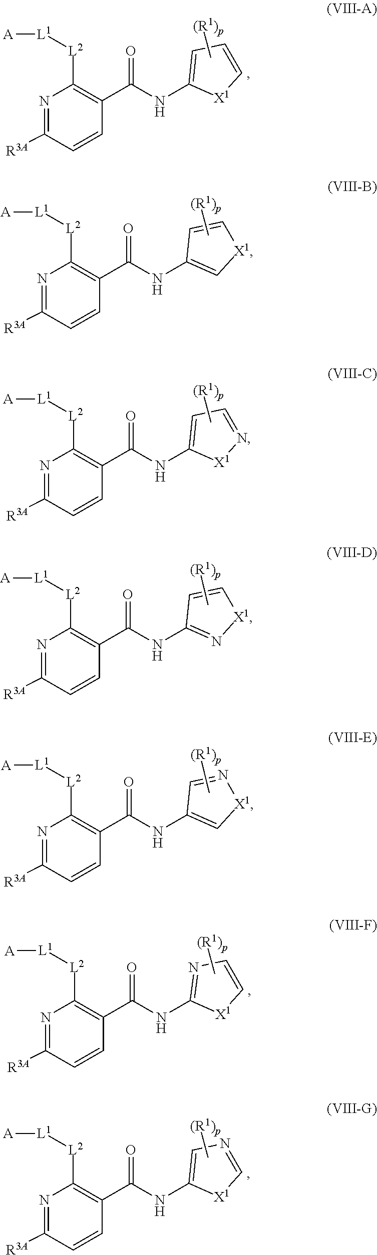

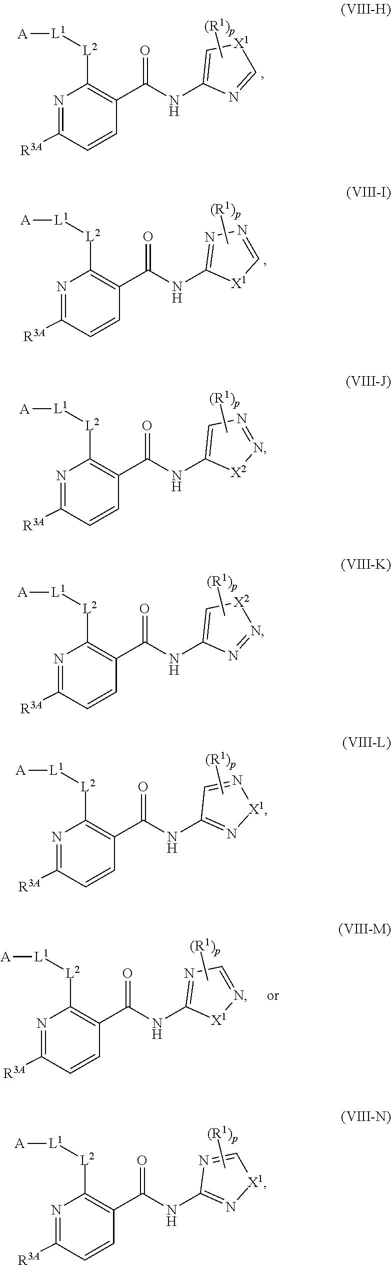

or a pharmaceutically acceptable salt thereof, wherein:
R¹, R^{3A}, A, L, L², p and R^{1A} are defined as in Formula (I);
X¹ is O, S or NR^{1A}; and
X² is O, S or NR^{1A}.
   E21. The heterobifunctional compound of embodiment E19 or E20, having the structure:

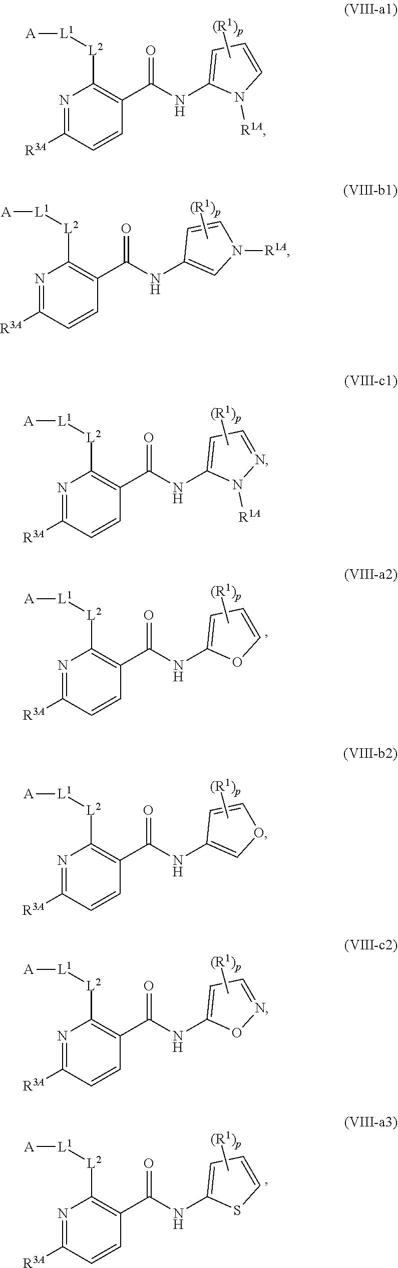

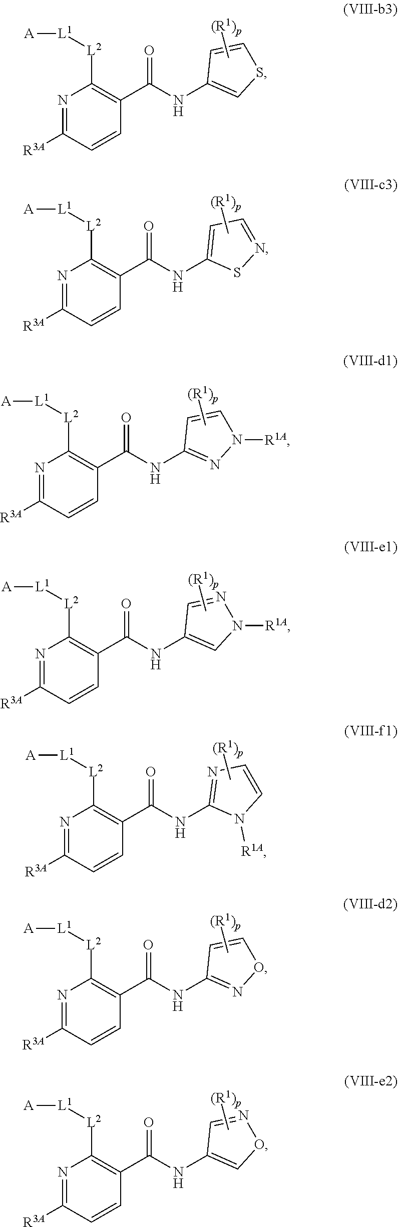

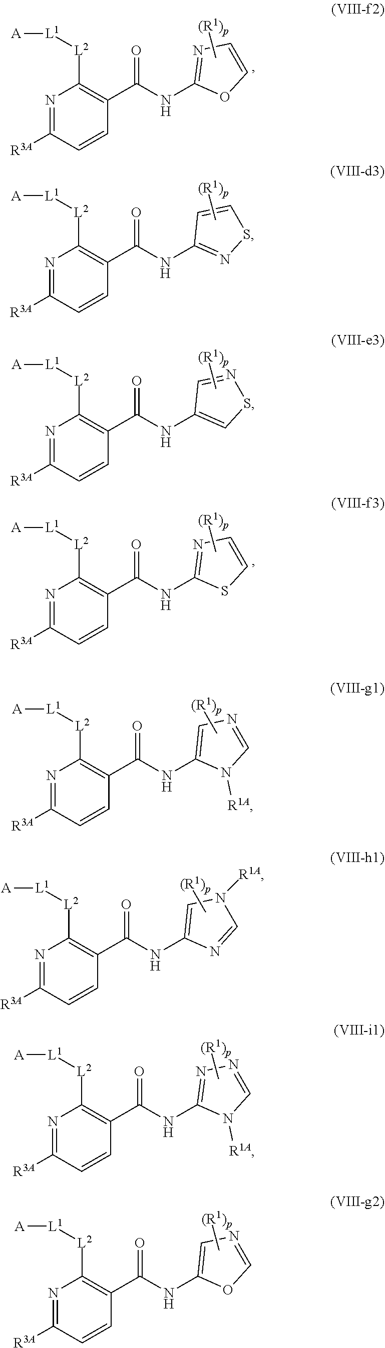

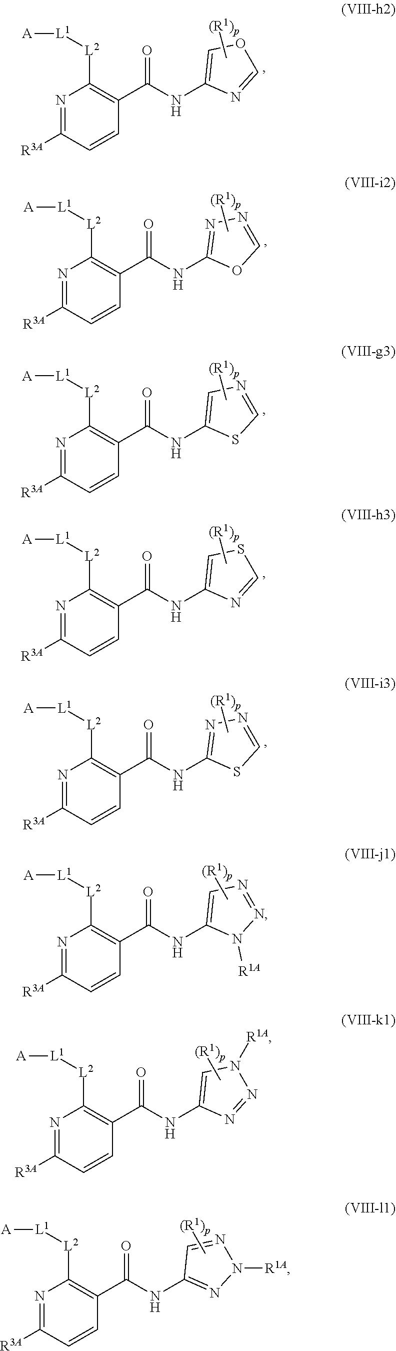

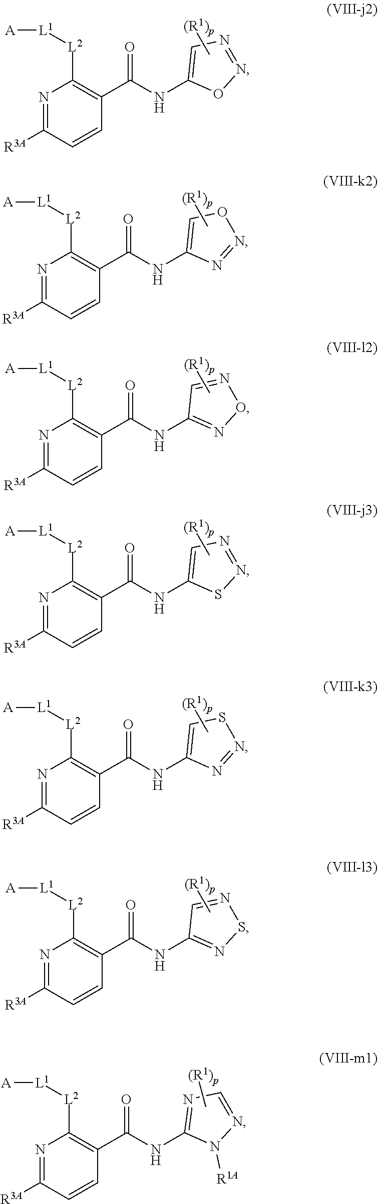

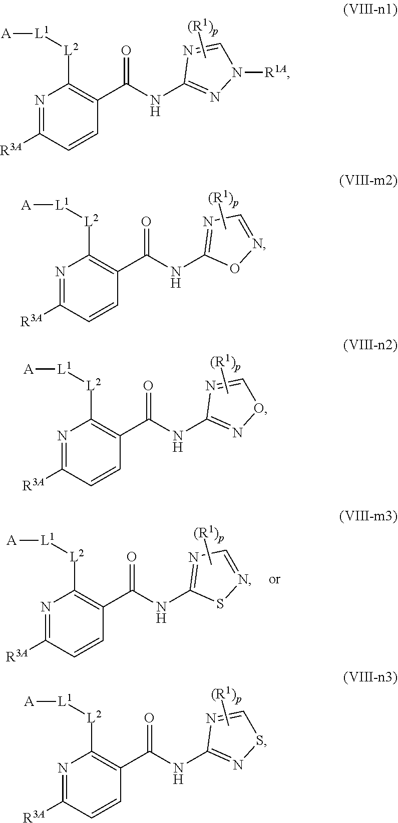

or a pharmaceutically acceptable salt thereof, wherein:
R¹, R^{3A}, A, L¹, L², p and R^{1A} are defined as in Formula (I).
   E22. The heterobifunctional compound of embodiment E4, E19, E20 or E21, having the structure:

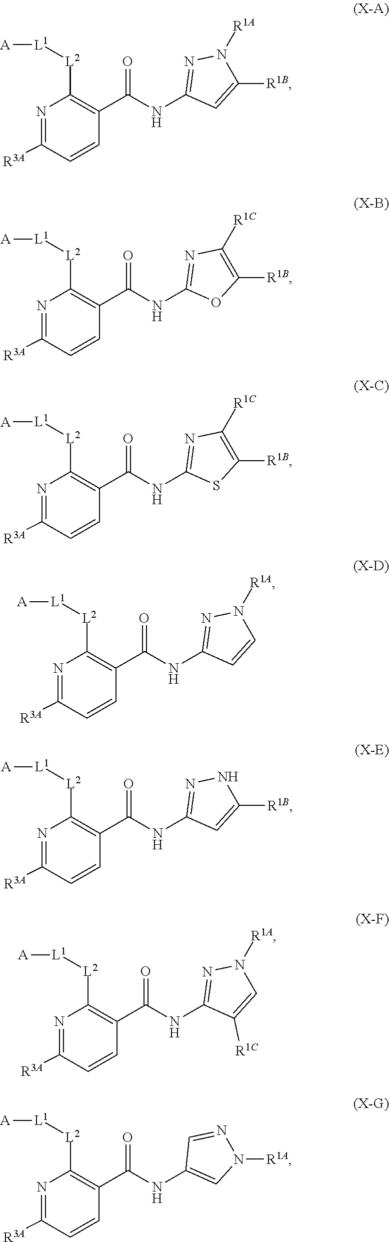

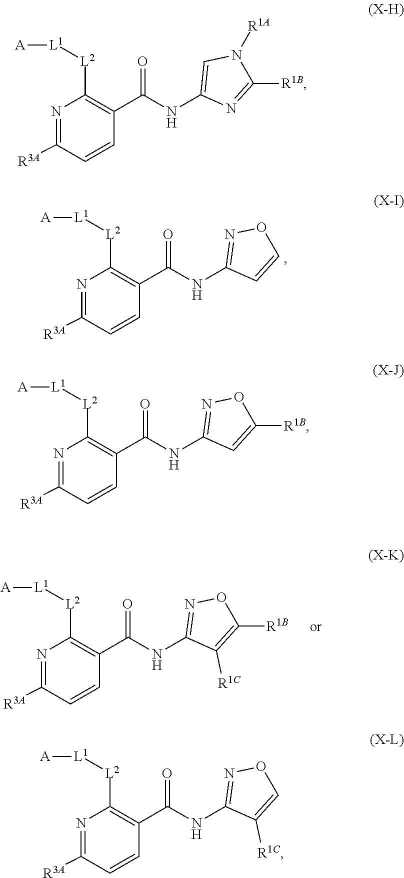

or a pharmaceutically acceptable salt thereof, wherein:
R^{1A}, R^{1B}, R^{1C}, R^{3A}, A, L¹ and L² are defined as in Formula (I).
   E23. The heterobifunctional compound of any one of embodiments E1 to E22, wherein A is a PBM having the structure of Formula (A):

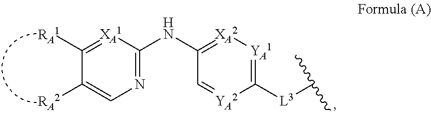

or a pharmaceutically acceptable salt thereof, wherein:
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, optionally substituted C₆-C₁₂ aryl, or optionally substituted 5-13 membered heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or 3-8 membered heterocyclyl; or
R_{A}¹ and R_{A}², together with the atom(s) to which they are attached optionally form an optionally substituted C₃-C₈ cycloalkyl, 3-8 membered heterocyclyl, C₆-C₁₂ aryl or 5-13 membered heteroaryl;
L³ is a divalent group selected from —R_{A}^{3A}-R_{A}^{3B}—, wherein R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₂ cycloalkylene, 3-12 membered heterocyclene, C₆-C₁₂ arylene, or 5-13 membered heteroarylene, provided two —O— and/or —S— are not contiguous;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl;
R_{A}⁵ and R_{A}⁶ are independently selected from hydrogen, C₁-C₈ alkyl, C₁-C₈ haloalkyl, C₁-C₈alkoxyalkyl, C₁-C₈ heteroalkyl, C₃-C₈ cycloalkyl, 3-8 membered heterocyclyl, C₆-C₁₂ aryl, or 5-13 membered heteroaryl; or
R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl or 5-13 membered heteroaryl; or R_{A}⁸ and R_{A}⁹ together with the atom(s) to which they are connected optionally form a 3-20 membered heterocyclyl ring.
   E24. The heterobifunctional compound of embodiment E23, wherein A is a PBM of Formula (A) having the structure of Formula (A1), (A2), or (A3):

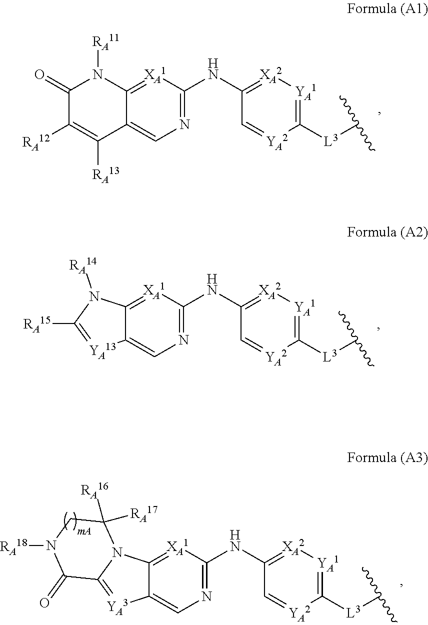

or a pharmaceutically acceptable salt thereof, wherein:
Y_{A}³ is CR_{A}¹⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or 3-8 membered heterocyclyl, C₆-C₁₂ aryl, or 5-13 membered heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹, wherein R_{A}²⁰ and R_{A}²¹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or 3-8 membered heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or 3-8 membered heterocyclyl;
R_{A}¹⁶ and R_{A}¹⁷ are each independently selected from hydrogen, C₁-C₇ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, 3-8 membered heterocyclyl, C₆-C₁₂ aryl, or 5-13 membered heteroaryl; or
R_{A}¹⁶ and R_{A}¹⁷, together with the atom(s) to which they are connected optionally form C₃-C₈ cycloalkyl, or 3-8 membered heterocyclyl; and
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈alkenyl, C₂-C₈ alkynyl, C₃-C₈ cycloalkyl, or 3-8 membered heterocyclyl; and
m_{A} is 0, 1, or 2.
   E25. The heterobifunctional compound of embodiment E23, wherein A is a PBM of Formula (A) having the structure of Formula (A4):

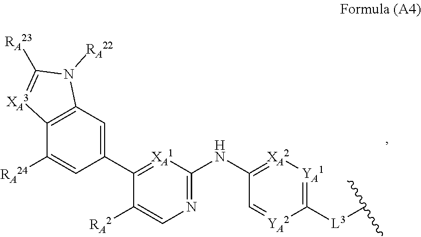

or a pharmaceutically acceptable salt thereof, wherein:
X_{A}³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, C₆-C₁₂ aryl, or 3-13 membered heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.
   E26. The heterobifunctional compound of any one of embodiments E1 to E23, wherein A is a PBM of Formula (A) having the structure of any of Formulae (A5), (A6), (A7), (A8) or (A9):

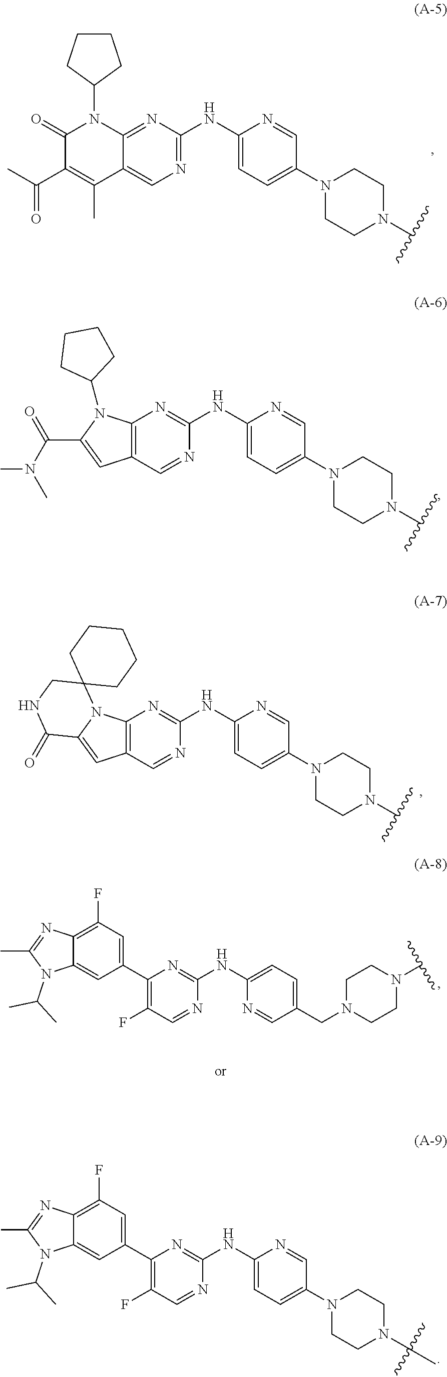 [SMILES: *N1CCN(c2ccc(Nc3ncc4c(C)c(C(C)=O)c(=O)n(C5CCCC5)c4n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc(F)c(-c4cc(F)c5nc(C)n(C(C)C)c5c4)n3)nc2)CC1]

E27. The heterobifunctional compound of embodiment E4, having the structure of Formula (XI):

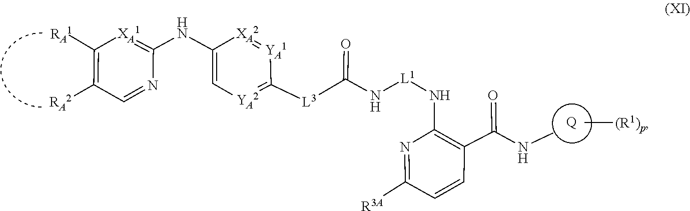

or a pharmaceutically acceptable salt thereof, wherein:
R¹, R^{3A}, L¹, Q and p are defined as for Formula (I);
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, optionally substituted C₆-C₁₂ aryl, or optionally substituted 5-13 membered heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or 3-8 membered heterocyclyl; or
R_{A}¹ and R_{A}², together with the atom(s) to which they are attached optionally form an optionally substituted C₃-C₈ cycloalkyl, 3-8 membered heterocyclyl, C₆-C₁₂ aryl or 5-13 membered heteroaryl;
L³ is a divalent group selected from —R_{A}^{3A}-R_{A}^{3B}—, wherein R_{A}^{3A} and R_{A}^{3B} are each independently a bond, —O—, —S—, —NR_{A}⁷-, —C(═O)—, —C(═O)NR_{A}⁷-, —S(═O)—, —S(═O)NR_{A}⁷-, —S(═O)₂—, —S(═O)₂NR_{A}⁷-, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₂ cycloalkylene, 3-12 membered heterocyclene, C₆-C₁₂ arylene, or 5-13 membered heteroarylene, provided two —O— and/or —S— are not contiguous;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl;
R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl; or
R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl or 5-13 membered heteroaryl; or
R_{A}⁸ and R_{A}⁹ together with the atom(s) to which they are connected optionally form a 3-20 membered heterocyclyl ring.
   E28. The heterobifunctional compound of embodiment E27, having the structure of Formula (XI-A), (XI-B), (XI-C) or (XI-D):

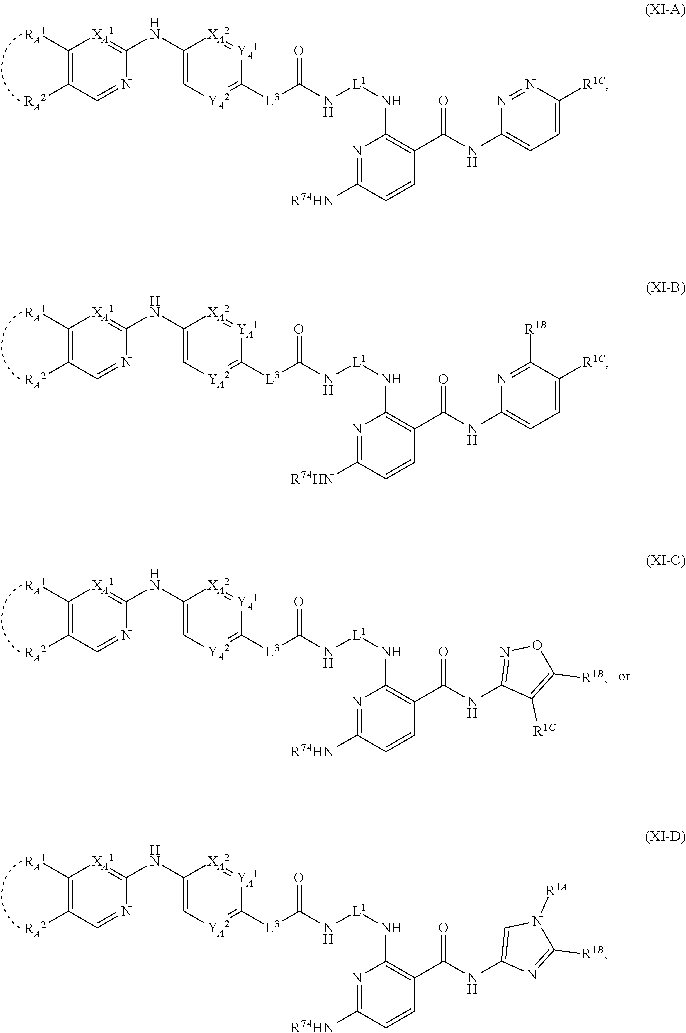

or a pharmaceutically acceptable salt thereof, wherein:
R^{1A}, R^{1B} and R^{1C} are defined as for Formula (I).
   E29. The heterobifunctional compound of embodiment E4, having the structure of Formula (XII):

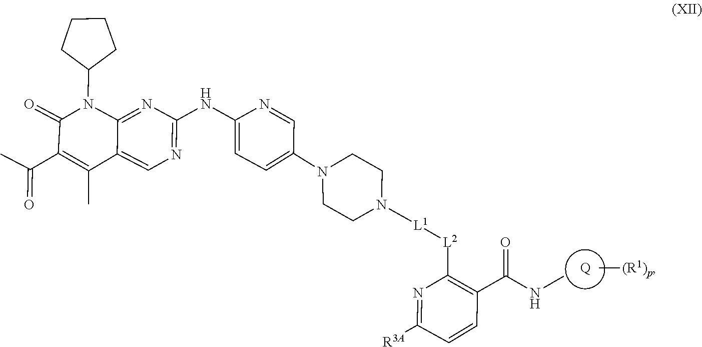

or a pharmaceutically acceptable salt thereof, wherein:
R¹, R^{3A}, L¹, L², Q and p are defined as for Formula (I).
   E30. The heterobifunctional compound of embodiment E29, having the structure of Formula (XII-A), (XII-B), (XII-C) or (XII-D):

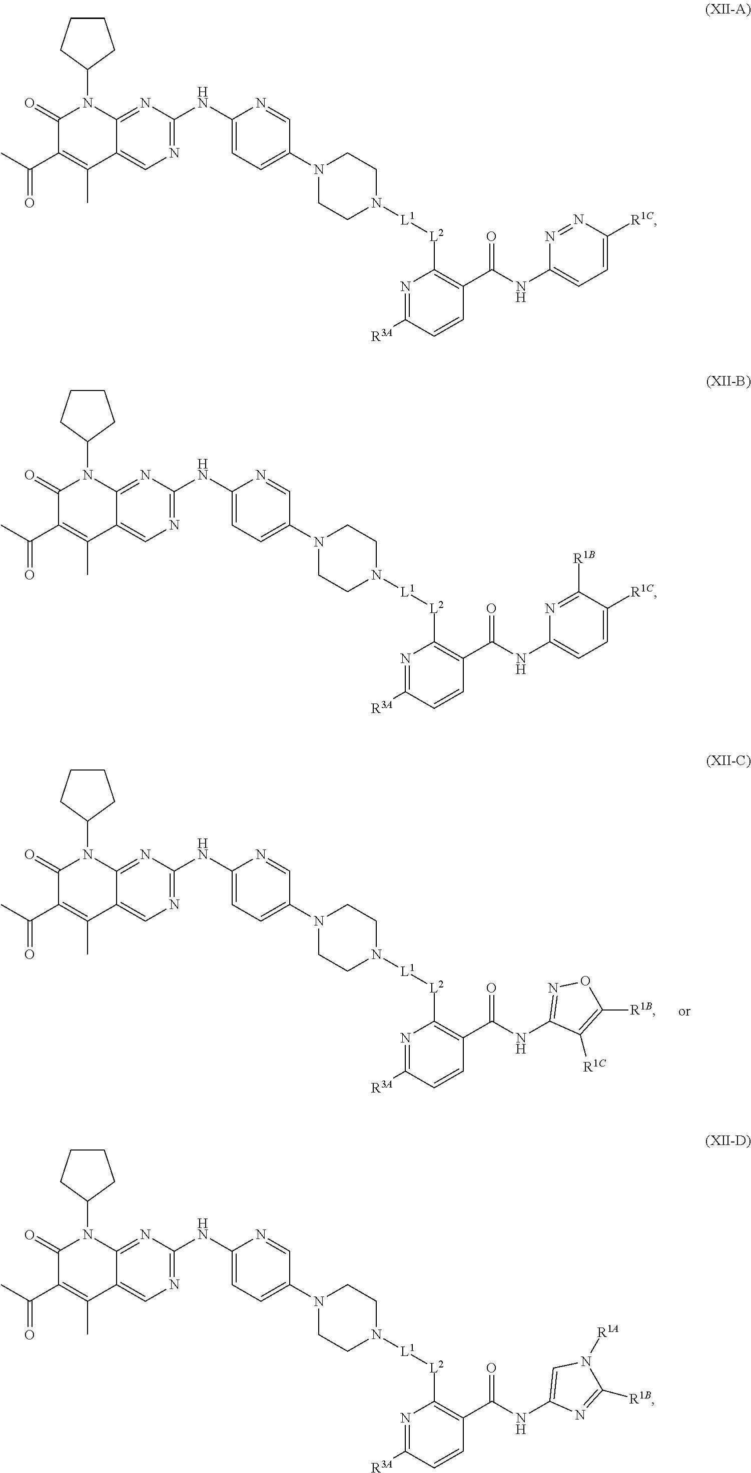

or a pharmaceutically acceptable salt thereof, wherein:
R^{1A}, R^{1B}, R^{1C}, R^{3A}, L¹ and L² are defined as for Formula (I).
   E31. The heterobifunctional compound of any one of embodiments E1 to E30, or a pharmaceutically acceptable salt thereof, wherein L¹ is a bond or a bivalent linker of Formula (L-1):

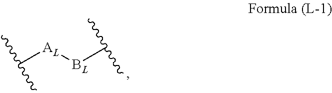

wherein:
each A_{L} and B_{L} is a bivalent moiety independently selected from the group consisting of a bond, R_{L}^{a}, R_{L}^{a}-R_{L}^{b}, R_{L}^{a}C(O)R_{L}^{b}, R_{L}^{a}C(O)OR_{L}^{b}, R_{L}^{a}OC(O)R_{L}^{b}, R_{L}^{a}C(O)N(R_{L}¹)R_{L}^{b}, R_{L}^{a}N(R_{L}¹)C(O)R_{L}^{b}, R_{L}^{a}C(S)N(R_{L}¹)R_{L}^{b}, R_{L}^{a}N(R_{L}¹)C(S)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)SO₂R_{L}^{b}, R_{L}^{a}N(R_{L}¹)R_{L}^{b}, and R_{L}^{a}N(R_{L}¹)C(O)N(R_{L}²)R_{L}^{b};
each R_{L}^{a} and R_{L}^{b} is independently selected from the group consisting of a bond, R_{L}^{r}, (C₁-C₈ alkylene)-R_{L}^{r}, R_{L}¹-(C₁-C₈ alkylene), (C₁-C₈ alkylene)-R_{L}^{r}-(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, where each said C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene moiety is optionally substituted by one or more R_{L}^{c};
each R_{L}^{r} is independently selected from the group consisting of C₃-C₁₂ cycloalkylene, 3-12 membered heterocyclene, C₆-C₁₂ arylene, and 5-13 membered heteroarylene, where each said C₃-C₁₂ cycloalkylene or 3-12 membered heterocyclene is optionally substituted by one or more R_{L}^{d}, and each said C₆-C₁₂ arylene or 5-13 membered heteroarylene is optionally substituted by one or more R_{L}^{e};
each R_{L}¹ and R_{L}² is independently selected from the group consisting of H, C₁-C₈ alkyl, C₂-C₈ alkenyl, C₂-C₈ alkynyl, C₃-C₂ cycloalkyl, 3-12 membered heterocyclyl, C₆-C₁₂ aryl, and 5-13 membered heteroaryl, where each said C₁-C₈ alkyl, C₂-C₈ alkenyl or C₂-C₈ alkynyl is optionally substituted by one or more R_{L}^{f}, each said C₃-C₁₂ cycloalkyl or 3-12 membered heterocyclyl is optionally substituted by one or more R_{L}^{g}, and each said C₆-C₁₂ aryl or 5-13 membered heteroaryl is optionally substituted by one or more R_{L}^{h}; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₁₂ carbocyclyl or 3-12 membered heterocyclyl;
each R_{L}^{e} and R_{L}^{f} is independently selected from the group consisting of F, OH, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl);
each R_{L}^{d} and R_{L}^{g} is independently selected from the group consisting of F, OH, C₁-C₄ alkyl, C₁-C₄ fluoroalkyl, C₃-C₆ cycloalkyl, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); and
each R_{L}^{e} and R_{L}^{h} is independently selected from the group consisting of halo, OH, C₁-C₄ alkyl, C₁-C₄ fluoroalkyl, C₃-C₆ cycloalkyl, C₁-C₄ alkoxy, C₃-C₆ cycloalkoxy, oxo, CN, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); or
two R_{L}^{c}, R_{L}^{d}, R_{L}^{e}, R_{L}^{f}, R_{L}^{g}, and R_{L}^{h} together with the atom(s) to which they are attached optionally form a C₃-C₁₂ carbocyclyl or 3-12 membered heterocyclyl.
   E32. The heterobifunctional compound or salt of embodiment E31, wherein A_{L} is a bivalent moiety selected from the group consisting of R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b} and R_{L}^{a}N(R_{L}¹)R_{L}^{b}.
   E33. The heterobifunctional compound or salt of embodiment E31 or E32, wherein:
A_{L} is R_{L}^{a}C(O)NHR_{L}^{b}, R_{L}^{a} is C₁-C₄ alkylene, and R_{L}^{b} is C₁-C₄ alkylene;
A_{L} is R_{L}^{a}C(O)NHR_{L}^{b}, R_{L}^{a} is(C₁-C₄ alkylene)-R_{L}^{r}, and R_{L}^{b} is C₁-C₄ alkylene;
A_{L} is R_{L}^{a}C(O)NHR_{L}^{b}, R_{L}^{a} is C₁-C₄ alkylene, and R_{L}^{b} is R_{L}^{r}-(C₁-C₄ alkylene);
A_{L} is R_{L}^{a}C(O)NHR_{L}^{b}, R_{L}^{a} is a bond, and R_{L}^{b} is(C₁-C₄ alkylene)-R_{L}^{r}-(C₁-C₄ alkylene);
A_{L} is R_{L}^{a}C(O)NHR_{L}^{b}, R_{L}^{a} is C₁-C₄ alkylene, and R_{L}^{b} is a bond;
A_{L} is R_{L}^{a}C(O)NHR_{L}^{b}, R_{L}^{a} is a bond, and R_{L}^{b} is C₁-C₄ alkylene;
A_{L} is R_{L}^{a}C(O)NHR_{L}^{b}, R_{L}^{a} is a bond, and R_{L}^{b} is a bond;
A_{L} is R_{L}^{a}N(R_{L}¹)C(O)R_{L}^{b}, R_{L}^{a} is C₁-C₄ alkylene, and R_{L}^{b} is C₁-C₄ alkylene;
A_{L} is R_{L}^{a}N(R_{L}¹)C(O)R_{L}^{b}, R_{L}^{a} is C₁-C₄ alkylene, and R_{L}^{b} is a bond;
A_{L} is R_{L}^{a}N(R_{L}¹)C(O)R_{L}^{b}, R_{L}^{a} is a bond, and R_{L}^{b} is C₁-C₄ alkylene;
A_{L} is R_{L}^{a}N(R_{L}¹)C(O)R_{L}^{b}, R_{L}^{a} is a bond, and R_{L}^{b} is a bond;
A_{L} is R_{L}^{a}NHC(O)R_{L}^{b}, R_{L}^{a} is C₁-C₄ alkylene, and R_{L}^{b} is C₁-C₄ alkylene;
A_{L} is R_{L}^{a}C(O)R_{L}^{b}, R_{L}^{a} is (C₁-C₄ alkylene)-R_{L}^{r}, and R_{L}^{b} is a bond;
A_{L} is R_{L}^{a}C(O)R_{L}^{b}, R_{L}^{a} is C₁-C₄ alkylene, and R_{L}^{b} is R_{L}^{r}—(C₁-C₄ alkylene);
A_{L} is R_{L}^{a}C(O)R_{L}^{b}, R_{L}^{a} is C₁-C₄ alkylene, and R_{L}^{b} is C₁-C₄ alkylene;
A_{L} is R_{L}^{a}C(O)R_{L}^{b}, R_{L}^{a} is C₁-C₄ alkylene, and R_{L}^{b} is a bond;
A_{L} is R_{L}^{a}C(O)R_{L}^{b}, R_{L}^{a} is a bond, and R_{L}^{b} is C₁-C₄ alkylene;
A_{L} is R_{L}^{a}C(O)R_{L}^{b}, R_{L}^{a} is a bond, and R_{L}^{b} is a bond;
A_{L} is R_{L}^{a}-R_{L}^{b}, R_{L}^{a} is C₁-C₄ alkylene, and R_{L}^{b} is C₁-C₄ alkylene;
A_{L} is R_{L}^{a}-R_{L}^{b}, R_{L}^{a} is (C₁-C₄ alkylene)-R_{L}^{r}, and R_{L}^{b} is C₁-C₄ alkylene;
A_{L} is R_{L}^{a}NHR_{L}^{b}, R_{L}^{a} is C₁-C₈ alkylene, and R_{L}^{b} is a bond;
A_{L} is R_{L}^{a}NHR_{L}^{b}, R_{L}^{a} is C₁-C₄ alkylene, and R_{L}^{b} is C₁-C₄ alkylene; or
A_{L} is R_{L}^{a}NHR_{L}^{b}, R_{L}^{a} is a bond, and R_{L}^{b} is C₁-C₈ alkylene.
   E34. The heterobifunctional compound or salt of any one of embodiments E31 to E33, wherein B_{L} is a bivalent moiety selected from the group consisting of a bond, R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b} and R_{L}^{a}N(R_{L}¹)R_{L}^{b}.
   E35. The heterobifunctional compound or salt of embodiment E34, wherein B_{L} is a bivalent moiety selected from the group consisting of a bond, R_{L}^{a}, R_{L}^{a}OR_{L}^{b} and R_{L}^{a}N(R_{L}¹)R_{L}^{b}.
   E36. The heterobifunctional compound or salt of embodiment E35, wherein B_{L} is R_{L}^{a}OR_{L}^{b}, R_{L}^{a} isa bond, and R_{L}^{b} is R_{L}^{r}, (C₁-C₈ alkylene)-R_{L}^{r}, or R_{L}^{r}-(C₁-C₈ alkylene).
   E37. The heterobifunctional compound or salt of embodiment E35, wherein B_{L} is R_{L}^{a}N(R_{L})R_{L}^{b}, R_{L}^{a} is a bond, and R_{L}^{b} is R_{L}^{r}, (C₁-C₈ alkylene)-R_{L}^{r}, or R_{L}^{r}-(C₁-C₈ alkylene).
   E38. The heterobifunctional compound or salt of embodiment E35, wherein B_{L} is R_{L}^{a}, where R_{L}^{a} is R_{L}^{r}, (C₁-C₈ alkylene)-R_{L}^{r}, R_{L}^{r}—(C₁-C₈ alkylene) or (C₁-C₈ alkylene)-R_{L}^{r}-(C₁-C₈ alkylene).
   E39. The heterobifunctional compound or salt of embodiment E38, wherein B_{L} is R_{L}^{a}, where R_{L}^{a} is R_{L}^{r}, (C₁-C₄ alkylene)-R_{L}^{r}, R_{L}^{T}-(C₁-C₄ alkylene) or (C₁-C₄ alkylene)-R_{L}^{r}-(C₁-C₄ alkylene).
   E40. The heterobifunctional compound or salt of any one of embodiments E31 to E39, wherein at least one R_{L}^{r} moiety in A_{L} or B_{L} is C₃-C₁₂ cycloalkylene or 3-12 membered heterocyclene, each optionally substituted by one or more R_{L}^{d}.
   E41. The heterobifunctional compound or salt of any one of embodiments E31 to E39, wherein at least one R_{L}^{r} moiety in A_{L} or B_{L} is C₆-C₁₂ arylene or 5-13 membered heteroarylene, each optionally substituted by one or more R_{L}^{e}.
   E42. The heterobifunctional compound or salt of any one of embodiments E31 to E41, wherein L¹ comprises one or more R_{L}^{r} moieties selected from the group consisting of Formula (L-2), Formula (L-3), Formula (L-4), Formula (L-5) and Formula (L-6):

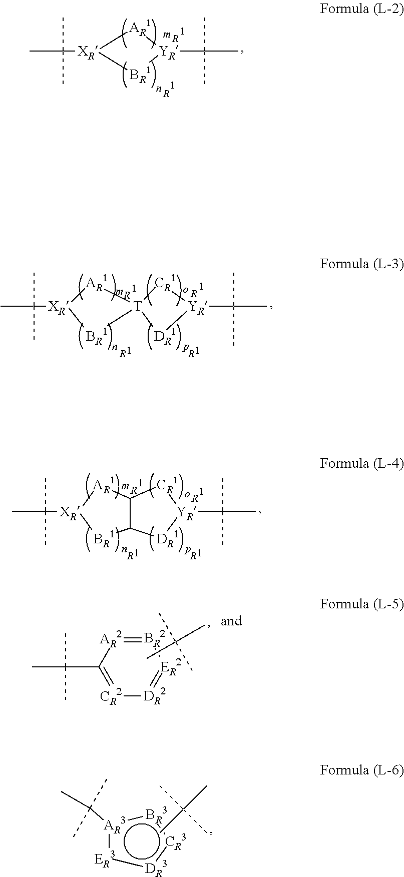

wherein:
X_{R}′ and Y_{R}′ are independently selected from N and CR_{R}^{b};
A_{R}¹, B_{R}¹, C_{R}¹ and D_{R}¹, at each occurrence, are independently selected from a bond, O, CO, SO, SO₂, C(O)NR_{R}^{b}, S(O)₂NR_{R}^{b}, NR_{R}^{b} and CR_{R}^{b}R_{R}^{c};
A_{R}², B_{R}², C_{R}², D_{R}², and E_{R}², at each occurrence, are independently selected from N and CR_{R}^{b};
A_{R}³, at each occurrence, is independently selected from N and C, and B_{R}³, C_{R}³, D_{R}³, and E_{R}³, at each occurrence, are independently selected from N, O, S, NR_{R}^{b} and CR_{R}^{b};
R_{R}^{b} and R_{R}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylamino C₁-C₈ alkyl, optionally substituted 3-12 membered carbocyclyl, optionally substituted 3-12 membered cycloalkoxy, optionally substituted 3-12 membered carbocyclylamino, optionally substituted 4-12 membered heterocyclyl, optionally substituted C₆-C₁₂ aryl, and optionally substituted 5-13 membered heteroaryl; or
two R_{R}^{b} and R_{R}^{c} together with the atom(s) to which they are attached optionally form a C₃-C₁₂ carbocyclyl or 3-12 membered heterocyclyl; and
m_{R}¹, n_{R}¹, o_{R}¹ and p_{R}¹ are independently selected from 0, 1, 2, 3, 4 and 5.
   E43. The heterobifunctional compound or salt of embodiment E42, wherein R_{L}^{r} is selected from the group consisting of:

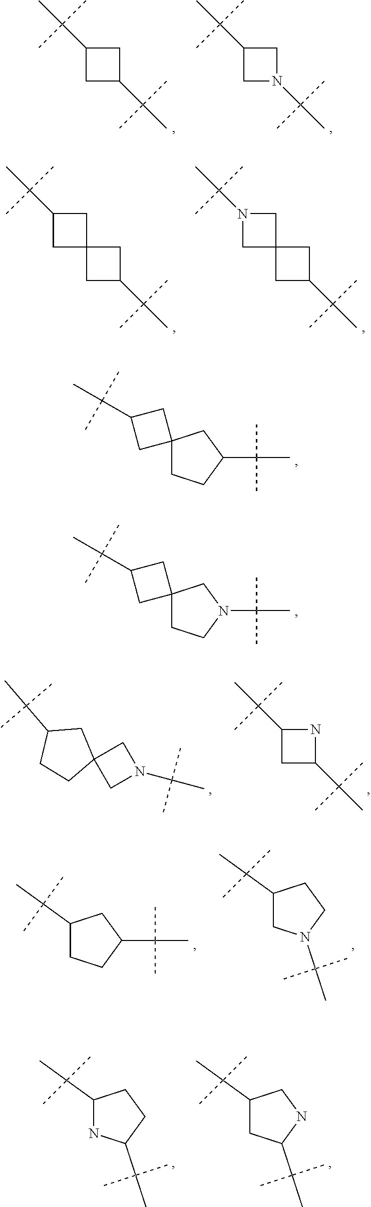

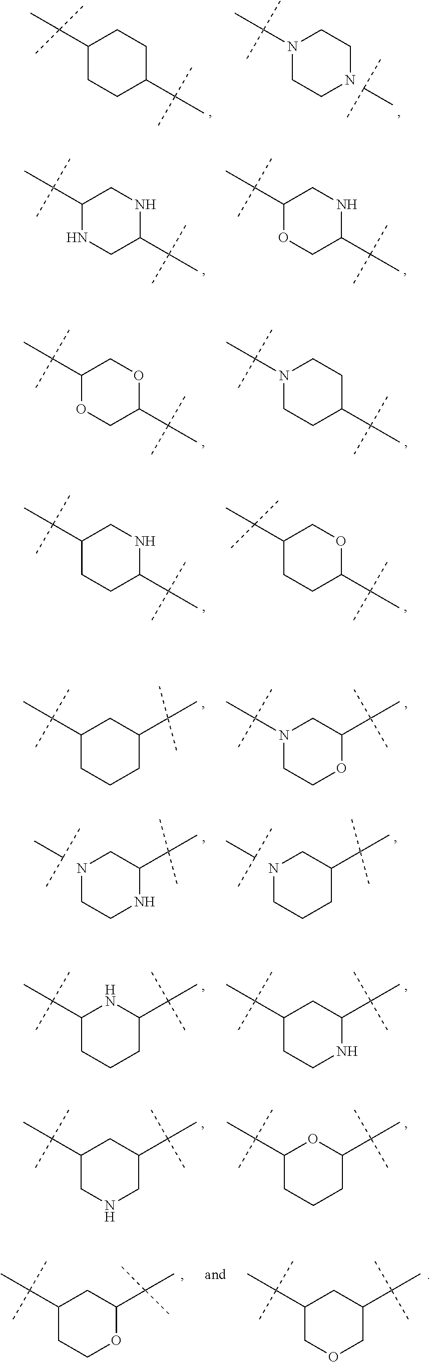

E44. The heterobifunctional compound or salt of embodiment E43, wherein R_{L}^{r} is selected from the group consisting of:

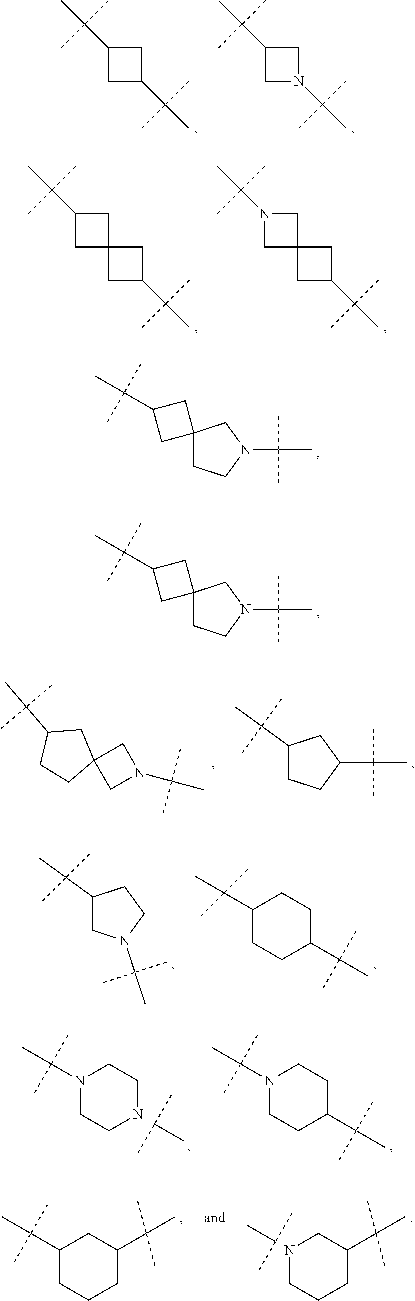

E45. The heterobifunctional compound or salt of any one of embodiments E31 to E35, wherein B_{L} is a bond.
E46. The heterobifunctional compound or salt of any one of embodiments E31 to E35, wherein B_{L} is R_{L}^{T}.
E47. The heterobifunctional compound of any one of embodiments E1 to E30, or a pharmaceutically acceptable salt thereof, wherein L¹ is a bivalent linker selected from the group consisting of:

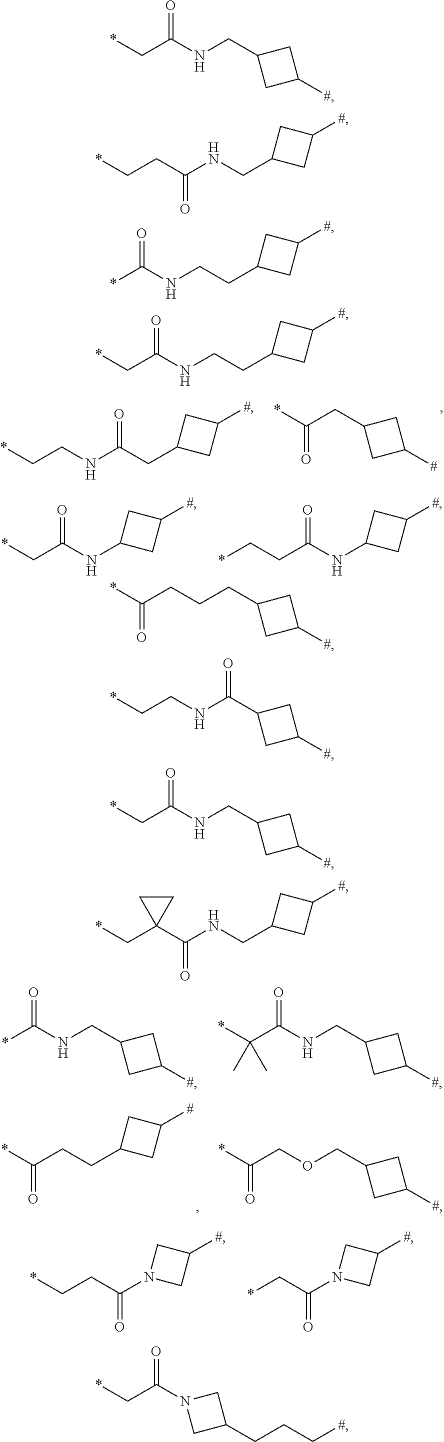

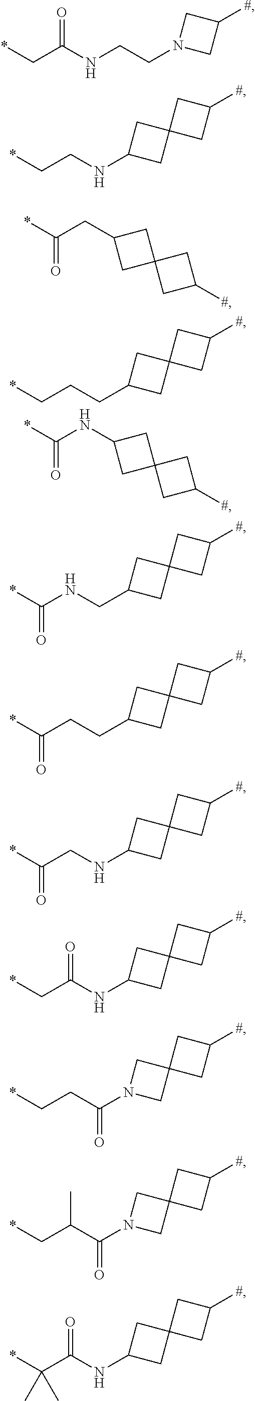

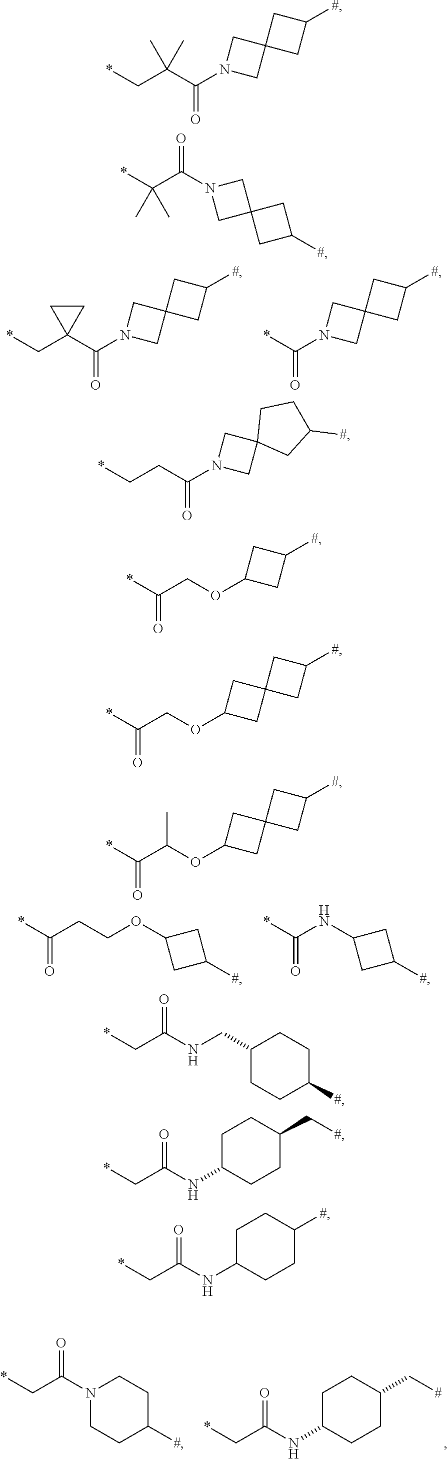

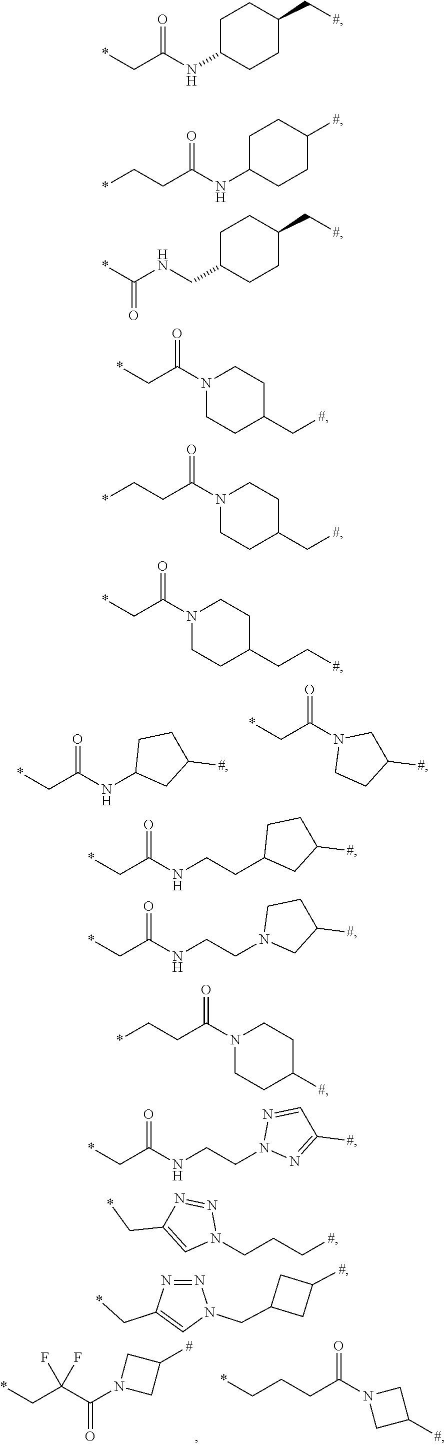

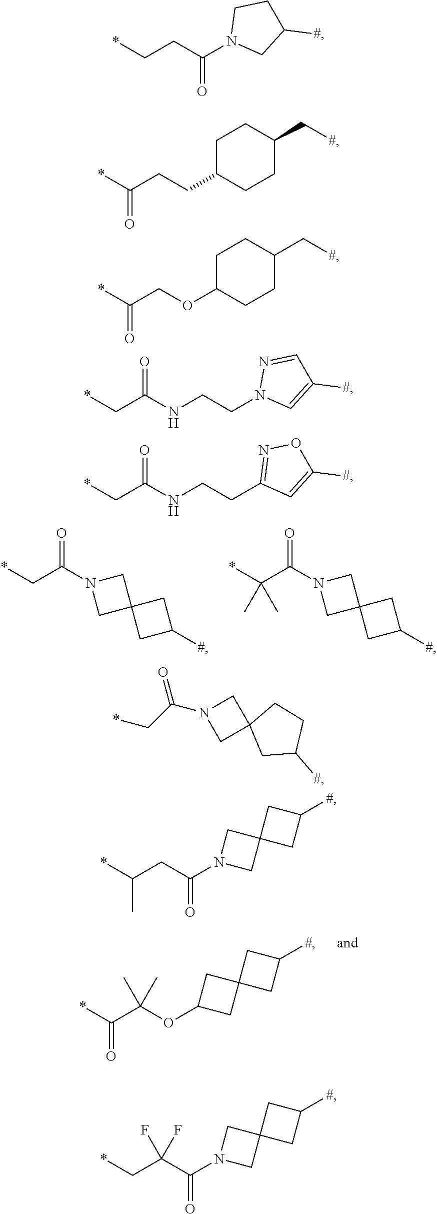

where #is the point of attachment to L² and * is the point of attachment to A.
   E48. The heterobifunctional compound of embodiment E47, or a pharmaceutically acceptable salt thereof, wherein L¹ is a bivalent linker selected from the group consisting of:

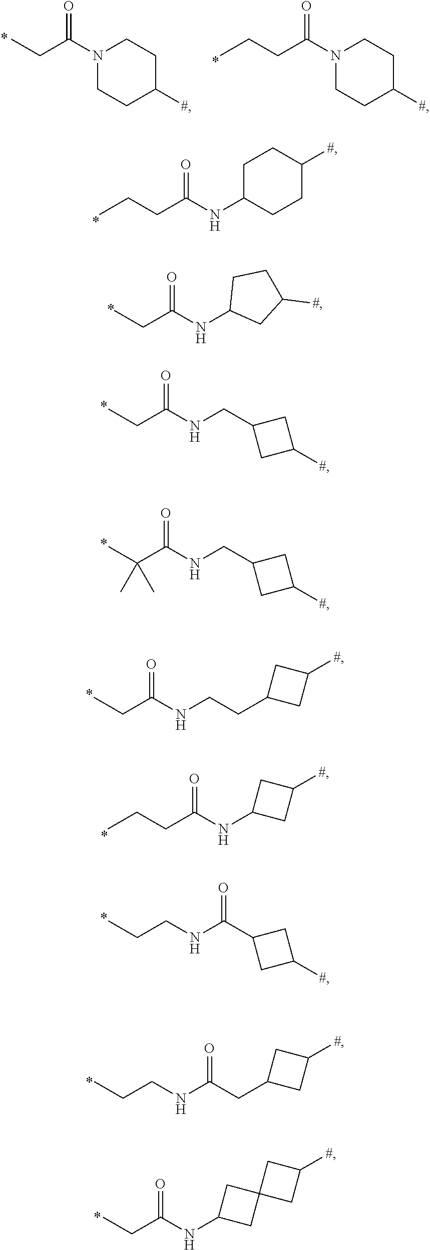

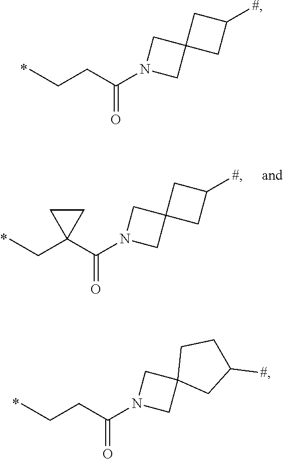

where #is the point of attachment to L² and * is the point of attachment to A.
   E49. A pharmaceutical composition comprising the compound of any one of embodiments E11 to E48, or a pharmaceutically acceptable salt thereof, and at least one pharmaceutically acceptable excipient.

In compounds of any of Formulae (I)-(IV), A is a PBM capable of binding to CCND, CDK4 and/or CDK6. In some embodiments, A is a PBM capable of binding to CDK4 and/or CDK6. In some embodiments, A is a PBM capable of binding to CDK4. In some embodiments, A is a PBM capable of binding to CDK6. In frequent embodiments, A is a PBM capable of binding to CDK4 and CDK6. In some embodiments of any of Formulae (I)-(IV), A is a PBM of any of Formulae (A), (A1), (A2), (A3) or (A4), as further described herein. In some embodiments, A is a PBM of any of Formulae (A5), (A6), (A7), (A8) or (A9), as described herein. In some preferred embodiments, A is a PBM of Formula (A5).

In compounds of any of Formulae (I)-(IV), L¹ is a bond or a bivalent linker. In some embodiments, L¹ is a bond or a bivalent linker of Formula (L), as further described herein. In some preferred embodiments, L¹ is a bond or a bivalent linker of Formula (L-1), as further described herein. In some embodiments, L¹ is a bond or a bivalent linker of Formula (J), as further described herein.

In compounds of any of Formulae (I)-(IV), L² is a bond, —NH—, —N(C₁-C₃ alkyl)-, —NHC(O)—, —N(C₁-C₃ alkyl)C(O)—, —C(O)NH—, —C(O)N(C₁-C₃ alkyl)-, —O—, —C₁-C₄-alkylene-, —C₂-C₄-alkenylene- or —C₂-C₄-alkynylene-. In some embodiments, L² is —NH—, —N(C₁-C₃ alkyl)-, —NHC(O)— or —N(C₁-C₃ alkyl)C(O)—. In some such embodiments, L² is —NH— or —N(C₁-C₃ alkyl)-. In preferred embodiments, L² is —NH—. In some embodiments, L² is —N(C₁-C₃ alkyl)-. In other embodiments, L² is —C(O)NH— or —C(O)N(C₁-C₃ alkyl)-. In some embodiments, L² is a bond, —C₁-C₄-alkylene-, —C₂-C₄-alkenylene- or —C₂-C₄-alkynylene-. In some embodiments, L² is a bond or —C₁-C₄-alkylene-. In some such embodiments, L² is a bond. In other such embodiments, L² is —C₁-C₄-alkylene-.

In compounds of any of Formulae (I)-(IV), Q is C₆-C₁₀ aryl, 5-10 membered heteroaryl, C₃-C₁₀ cycloalkyl or 4-10 membered heterocyclyl. In some embodiments, Q is C₆-C₁₀ aryl or 5-10 membered heteroaryl. In some embodiments, Q is C₆-aryl (i.e., phenyl) or 5-6 membered heteroaryl. In some embodiments, Q is phenyl. In some embodiments, Q is a 5-6 membered heteroaryl. In some preferred embodiments, Q is a 5-6 membered heteroaryl selected from the group consisting of pyridine, pyridazine, imidazole, isoxazole, oxazole and pyrazole. In some preferred embodiments, Q is selected from the group consisting of phenyl, pyridine, pyridazine, imidazole, isoxazole, oxazole and pyrazole. In some embodiments, Q is a 6 membered heteroaryl. In some such embodiments, Q is a 6 membered heteroaryl selected from the group consisting of pyridine, pyridazine, pyrimidine, and pyrazine. In some preferred embodiments, Q is pyridine or pyridazine. In some preferred embodiments, Q is pyridine-2-yl or pyridazine-3-yl. In some embodiments, Q is a 5 membered heteroaryl. In some such embodiments, Q is a 5 membered heteroaryl selected from the group consisting of pyrrole, pyrazole, imidazole, isoxazole, oxazole, isothiazole, thiazole, triazole, oxadiazole, and thiadiazole. In some preferred embodiments, Q is imidazole, isoxazole or pyrazole. In some such embodiments, Q is imidazole. In some such embodiments, Q is isoxazole. In some such embodiments, Q is oxazole. In some such embodiments, Q is pyrazole.

In compounds of any of Formulae (I)-(IV), Q is optionally substituted by p substituent groups R¹, where p is an integer from 0 to 4, and each R¹ is independently R^{1A}, R^{1B}, R^{1C}, R^{1D} or R^{1E}. In some embodiments, p is an integer from 1 to 3. In frequent embodiments, p is an integer from 1 to 2. In some embodiments, p is 1. In some embodiments, p is 2.

In frequent embodiments of any of Formulae (I)-(IV), each R^{1A} is an optional substituent group attached to a nitrogen atom of ring Q, and each R^{1B}, R^{1C}, R^{1D} or R^{1E} is an optional substituent group attached to a carbon atom of ring Q.

In compounds of any of Formulae (I)-(IV), each R^{1A} is independently H, C₁-C₆ alkyl, C₃-C₆ cycloalkyl, or C₁-C₆ heteroalkyl, where each said C₁-C₆ alkyl, C₃-C₆ cycloalkyl, or C₁-C₆ heteroalkyl is optionally substituted by one or more R^{4A}, as further described. In other embodiments, each optionally substituted moiety of R^{1A} is unsubstituted. In some such embodiments, each R^{1A} is independently H, C₁-C₆ alkyl, C₃-C₆ cycloalkyl, or C₁-C₆ heteroalkyl. In some embodiments, each R^{1A} is independently H, C₁-C₆ alkyl, or C₃-C₆ cycloalkyl. In some preferred embodiments, each R^{1A} is independently H or C₁-C₄ alkyl. In some such embodiments, each R^{1A} is independently H, CH₃, CH₂CH₃, CH₂CH₂CH₃ or CH(CH₃)₂. In some such embodiments, each R^{1A} is independently H, CH₃ or CH₂CH₃. In some preferred embodiments, each R^{1A} is independently C₃-C₆ cycloalkyl. In some such embodiments, each R^{1A} is independently cyclopropyl, cyclobutyl, cyclopentyl, or cyclohexyl. In some such embodiments, each R^{1A} is independently cyclopropyl or cyclobutyl.

In compounds of any of Formulae (I)-(IV), where present, each R^{4A} is independently C₁-C₆ alkoxy, oxo, OH, D, halo (preferably F), CN or NR^{8A}R^{8B}. In some embodiments when R^{4A} is NR_{8A}R^{8B}, each R^{8A} and R^{8B} is independently H, C₁-C₄ alkyl or C₃-C₆ cycloalkyl, or R^{8A} and R^{8B} are taken together with the N-atom to which they are attached to form a 4-6 membered heterocyclyl ring. In some such embodiments, each R^{8A} and R^{8B} is independently H or C₁-C₄ alkyl. In other embodiments, each NR^{8A}R^{8B} is independently NH₂, NH(C₁-C₄-alkyl) or N(C₁-C₄-alkyl)₂. In some preferred embodiments, each R^{4A} is independently C₁-C₄ alkoxy, OH, D, F, NH₂, NH(C₁-C₄-alkyl) or N(C₁-C₄-alkyl)₂.

In compounds of any of Formulae (I)-(IV), each R^{1B}, R^{1C}, R^{1D} and R^{1E} is independently H, C₁-C₆ alkyl, C₁-C₆ heteroalkyl, C₁-C₆ alkoxy, OH, D, halo, CN, NO₂, NR^{5A}R^{5B}, C(═O)R^{5A}, C(═O)OR^{5A}, OC(═O)R^{5A}, C(O)NR^{5A}R^{5B}, N(R^{5A})C(O)R^{5B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{4B}, and each said C₃-C₁₀ cycloalkyl, 4-10 membered heterocyclyl, C₆-C₁₀ aryl or 5-10 membered heteroaryl is optionally substituted by one or more R^{4C}, as further described.

In some embodiments, each R^{1B}, R^{1C}, R^{1D} and R^{1E} is independently H, C₁-C₆ alkyl, C₁-C₆ alkoxy, halo, CN, NR^{5A}R^{5B}, C₃-C₁₀ cycloalkyl, or C₃-C₁₀ cycloalkoxy, where each said C₁-C₆ alkyl or C₁-C₆ alkoxy is optionally substituted by one or more R^{4B} and each said C₃-C₁₀ cycloalkyl or C₃-C₁₀ cycloalkoxy is optionally substituted by one or more R^{4C}, as further described. In some embodiments, each optionally substituted moiety of R^{1B}, R^{1C}, R^{1D} and R^{1E} is unsubstituted. In some preferred embodiments, each R^{1B}R^{1C}, R^{1D} and R^{1E} is independently H, C₁-C₄ alkyl, C₁-C₄ alkoxy, halo, CN, NR^{5A}R^{5B}, C₃-C₆ cycloalkyl, or C₃-C₁₀ cycloalkoxy.

In some embodiments, when R^{1B}, R^{1C}, R^{1D} or R^{1E} is NR^{5A}R^{5B}, each R^{5A} and R^{5B} is independently H, C₁-C₆ alkyl or C₃-C₁₀ cycloalkyl, or R^{5A} and R^{5B} are taken together with the N-atom to which they are attached to form a 4-10 membered heterocyclyl ring. In some preferred embodiments, each R^{5A} and R^{5B} is independently H, C₁-C₄ alkyl or C₃-C₆ cycloalkyl, or R^{5A} and R^{5B} are taken together with the N-atom to which they are attached to form a 4-6 membered heterocyclyl ring.

In some embodiments, each R^{1B}, R^{1C}, R^{1D} and R^{1E} is independently H, CH₃, CH₂CH₃, CH₂CH₂CH₃, CH(CH₃)₂, OCH₃, F, Cl, CN, NH₂, NHCH₃, N(CH₃)₂, N-pyrrolidinyl, or cyclopropyl.

In some embodiments of any of Formulae (I)-(IV), where present, each R^{4B} is independently C₁-C₆ alkoxy, oxo, OH, D, halo (preferably F), CN or NR^{8A}R^{8B}; and each R^{4C} is independently C₁-C₄ alkyl, C₁-C₆ alkoxy, oxo, OH, D, halo (preferably F), CN or NR^{9A}R^{9B}. In some such embodiments, each R^{8A}, R^{8B}, R^{9A} and R^{9B} is independently H or C₁-C₆ alkyl. In some such embodiments, each R^{8A}, R^{8B}, R^{9A} and R^{9B} is independently H or C₁-C₄ alkyl. In other embodiments, each NR^{8A}R^{8B} and NR^{9A}R^{9B} is independently NH₂, NH(C₁-C₄-alkyl) or N(C₁-C₄-alkyl)₂. In some preferred embodiments of any of Formulae (I)-(IV), where present, each R^{4B} is independently C₁-C₄ alkoxy, OH, D, F, NH₂, NH(C₁-C₄-alkyl) or N(C₁-C₄-alkyl)₂. In some preferred embodiments of any of Formulae (I)-(IV), where present, each R^{4C} is independently C₁-C₄ alkyl, C₁-C₄ alkoxy, OH, D, F, NH₂, NH(C₁-C₄-alkyl) or N(C₁-C₄-alkyl)₂. In some such embodiments, each R^{4B} is independently C₁-C₄ alkoxy, OH, D, F, NH₂, NH(C₁-C₄-alkyl) or N(C₁-C₄-alkyl)₂, and each R^{4C} is independently C₁-C₄ alkyl, C₁-C₄ alkoxy, OH, D, F, NH₂, NH(C₁-C₄-alkyl) or N(C₁-C₄-alkyl)₂.

In some embodiments of any of Formulae (I)-(IV), at least one of R^{1B}, R^{1C}, R^{1D} and R^{1E} is NR^{5A}R^{5B} where each R^{5A} and R^{5B} is independently H, C₁-C₆ alkyl or C₃-C₁₀ cycloalkyl, or R^{5A} and R^{5B} are taken together with the N-atom to which they are attached to form a 4-10 membered heterocyclyl ring; and preferably, or C₃-C₆ cycloalkyl, or R^{5A} and R^{5B} are taken together with the N-atom to which they are attached to form a 4-6 membered heterocyclyl ring (e.g., an N-linked pyrrolidinyl, piperidinyl, piperazinyl or morpholinyl moiety).

In some preferred embodiments, each R^{1A} is independently H, C₁-C₄ alkyl or C₃-C₆ cycloalkyl, and each R^{1B}, R^{1C}, R^{1D} and R^{1E} is independently H, C₁-C₄ alkyl, C₁-C₄ alkoxy, halo, CN, NR^{5A}R^{5B} or C₃-C₆ cycloalkyl. In such embodiments, each R^{1A} is independently H, CH₃, CH₂CH₃, CH₂CH₂CH₃, CH(CH₃)₂, cyclopropyl or cyclobutyl, and each R^{1B}, R^{1C}, R^{1D} and R^{1E} is independently H, CH₃, CH₂CH₃, CH₂CH₂CH₃, CH(CH₃)₂, OCH₃, F, Cl, NH₂, NHCH₃, N(CH₃)₂, N-pyrrolidinyl or cyclopropyl.

In compounds of any of Formulae (I)-(IV), R² is H or C₁-C₃ alkyl. In preferred embodiments, R² is H.

In compounds of any of Formulae (I)-(III), the core pyridine ring is optionally substituted by q substituent groups R³, where q is an integer from 0 to 3 and each R³ is independently R^{3A}, R^{3B} or R^{3C}; or two R³ may be taken together with the atoms to which they are attached to form a fused C₃-C₁₀ cycloalkyl, 4-10 membered heterocyclyl, C₆-C₁₀ aryl or 5-10 membered heteroaryl, each optionally substituted by one or more R^{6A}. In some embodiments, q is an integer from 1 to 3. In some embodiments, q is an integer from 1 to 2. In some embodiments, q is 1. In some embodiments, q is 2.

In compounds of Formula (IV), the core pyridine ring is substituted on the indicated carbon atom by R^{3A}.

In compounds of any of Formulae (I)-(IV), each R^{3A}, R^{3B} and R^{3C} is independently C₁-C₆ alkyl, C₁-C₆ heteroalkyl, C₁-C₆ alkoxy, OH, halo, CN, NO₂, NR^{7A}R^{7B}, C(═O)R^{7A}, C(═O)OR^{7A}, OC(═O)R^{7A}, C(O)NR^{7A}R^{7B}, N(R^{7A})C(O)R^{7B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{6B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{6C}, as further described. In some embodiments, each R^{3A}, R^{3B} and R^{3C} is independently C₁-C₆ alkyl, C₁-C₆ alkoxy, halo, CN, NR^{7A}R^{7B}, C₃-C₁₀ cycloalkyl, or C₃-C₁₀ cycloalkoxy, where each said C₁-C₆ alkyl or C₁-C₆ alkoxy is optionally substituted by one or more R^{6B}, and each said C₃-C₁₀ cycloalkyl or C₃-C₁₀ cycloalkoxy is optionally substituted by one or more R^{6C}, as further described. In some embodiments, each optionally substituted moiety of R^{3A}, R^{3B} and R^{3C} is unsubstituted. In some such embodiments, each R^{3A}, R^{3B} and R^{3C} is independently C₁-C₄ alkyl, C₁-C₄ alkoxy, halo, CN, NR^{7A}R^{7B}, C₃-C₆ cycloalkyl, or C₃-C₁₀ cycloalkoxy.

In some embodiments of any of Formulae (I)-(IV), at least one of R^{3A}, R^{3B} or R^{3C} is NR^{7A}R^{7B}. In some such embodiments when R^{3A}, R^{3B} or R^{3C} is NR^{7A}R^{7B}, each R^{7A} and R^{7B} is independently H, C₁-C₆ alkyl or C₃-C₁₀ cycloalkyl, or R^{7A} and R^{7B} are taken together with the N-atom to which they are attached to form a 4-10 membered heterocyclyl ring. In some such embodiments, each R^{7A} and R^{7B} is independently H, C₁-C₄ alkyl or C₃-C₆ cycloalkyl, or R^{7A} and R^{7B} are taken together with the N-atom to which they are attached to form a 4-6 membered heterocyclyl ring.

In some preferred embodiments, each R^{3A}, R^{3B} and R^{3C} is independently H, C₁-C₄ alkyl, C₁-C₄ alkoxy, halo, CN, NR^{7A}R^{7B}, C₃-C₆ cycloalkyl, or C₃-C₁₀ cycloalkoxy. In some such embodiments, each R^{3A}, R^{3B} and R^{3C} is independently CH₃, CH₂CH₃, CH₂CH₂CH₃, CH(CH₃)₂, OCH₃, F, Cl, CN, NH₂, NHCH₃, NHCH(CH₃)₂, NH(c-butyl), NH(t-butyl), N(CH₃)₂, N-pyrrolidinyl, or cyclopropyl.

In some preferred embodiments of Formula (IV), R^{3A} is NR^{7A}R^{7B}. In some such embodiments, R^{3A} is NH₂, NHCH₃, NHCD₃, NHCH(CH₃)₂, NH(c-butyl), NH(t-butyl), N(CH₃)², or N-pyrrolidinyl.

In compounds of any of Formulae (I)-(III), where present, each R^{6A} is independently C₁-C₄ alkyl, C₁-C₆ alkoxy, oxo, OH, D, halo, CN or NR^{9A}R^{9B}. In compounds of any of Formulae (I)-(IV), where present, each R^{6B} is independently C₁-C₆ alkoxy, oxo, OH, D, halo, CN or NR^{9A}R^{9B}; and preferably, each R^{6B} is independently C₁-C₄ alkoxy, OH, D, F, NH₂, NH(C₁-C₄-alkyl) or N(C₁-C₄-alkyl)₂. In compounds of any of Formulae (I)-(IV), where present, each R^{6C} is independently C₁-C₄ alkyl, C₁-C₆ alkoxy, oxo, OH, D, halo, CN or NR^{9A}R^{9B}; and preferably, each R^{6C} is independently C₁-C₄ alkyl, C₁-C₄ alkoxy, OH, D, F, NH₂, NH(C₁-C₄-alkyl) or N(C₁-C₄-alkyl)₂. In some embodiments when any of R^{6A}, R^{1B} or R^{6C} is NR^{9A}R^{9B}, each R^{9A} and R^{9B} is independently H, C₁-C₆ alkyl or C₃-C₁₀ cycloalkyl, or R^{9A} and R^{9B} are taken together with the N-atom to which they are attached to form a 4-10 membered heterocyclyl ring. In some such embodiments, each R^{9A} and R^{9B} is independently H, C₁-C₄ alkyl or C₃-C₆ cycloalkyl, or R^{9A} and R^{9B} are taken together with the N-atom to which they are attached to form a 4-6 membered heterocyclyl ring.

In compounds of any of Formulae (I)-(IV), where present, each R^{5A}, R^{5B}, R^{7A}, R^{7B}, R^{8A}, R^{8B}, R^{9A} and R^{9B} is independently H, C₁-C₆ alkyl, C₁-C₆ heteroalkyl, C₃-C₁₀ cycloalkyl, 4-10 membered heterocyclyl, C₆-C₁₀ aryl or 5-10 membered heteroaryl, where each said C₁-C₆ alkyl or C₁-C₆ heteroalkyl is optionally substituted by one or more R^{10A}, and each said C₃-C₁₀ cycloalkyl, 4-10 membered heterocyclyl, C₆-C₁₀ aryl or 5-10 membered heteroaryl is optionally substituted by one or more R^{10B} or R^{5A} and R^{5B}, R^{7A} and R^{7B}, R^{8A} and R^{8B}, or R^{9A} and R^{9B} may be taken together with the N-atom to which they are attached to form a 4-10 membered heterocyclyl ring or 5-10 membered heteroaryl ring, where each said 4-10 membered heterocyclyl or 5-10 membered heteroaryl is optionally substituted by one or more R^{10C}.

In some such embodiments, where present, each R^{10A} is independently C₁-C₆ alkoxy, oxo, OH, D, halo, CN or NR^{11A}R^{11B}; preferably, each R^{10A} is independently C₁-C₄ alkoxy, OH, D, F, NH₂, NH(C₁-C₄-alkyl) or N(C₁-C₄-alkyl)₂. In some embodiments, where present, each R^{10B} and R^{10C} is independently C₁-C₄ alkyl, C₁-C₆ alkoxy, oxo, OH, D, halo, CN or NR^{12A}R^{12B}; preferably, each R^{10B} and R^{10C} is independently C₁-C₄ alkyl, C₁-C₄ alkoxy, F, Cl, CN, NH₂, NH(C₁-C₄-alkyl) or N(C₁-C₄-alkyl)₂. In some embodiments, where present, each R^{11A}, R^{11B}, R^{12A} and R^{12B} is independently H or C₁-C₄ alkyl.

In some embodiments of any of Formulae (I)-(IV), where present, each R^{5A}, R^{5B}, R^{7A}, R^{7B}, R^{8A}, R^{8B}, R^{9A} and R^{9B} is independently H, C₁-C₆ alkyl, or C₃-C₁₀ cycloalkyl; or R^{5A} and R^{5B}, R^{7A} and R^{7B}, R^{8A} and R^{8B}, or R^{9A} and R^{9B} may be taken together with the N-atom to which they are attached to form a 4-10 membered heterocyclyl. In some such embodiments, each R^{5A}, R^{5B}, R^{7A}, R^{7B}, R^{8A}, R^{8B}, R^{9A} and R^{9B} is independently H, C₁-C₄ alkyl, or C₃-C₆ cycloalkyl; or R^{5A} and R^{5B}, R^{7A} and R^{7B}, R^{8A} and R^{8B}, or R^{9A} and R^{9B} may be taken together with the N-atom to which they are attached to form a 4-6 membered heterocyclyl.

In some preferred embodiments of any of Formulae (I)-(IV), Q is selected from:

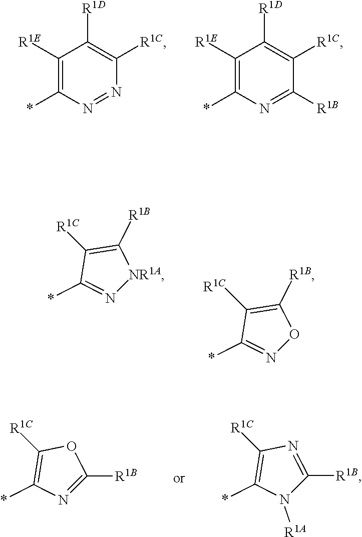

wherein:
*represents the point of attachment; and
R^{1A}, R^{1B}, R^{1C}, R^{1D} and R^{1E} are defined as in Formula (I).

In some such embodiments of any of Formulae (I)-(IV), Q is selected from:

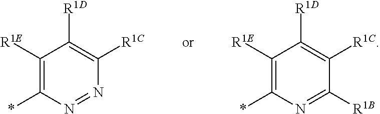

In some such embodiments of any of Formulae (I)-(IV), Q is selected from:

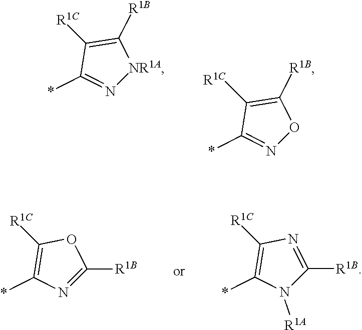

Embodiments described herein for any of Formulae (I)-(IV) are also applicable to compounds of any of Formulae (V)-(XII), to the extent such embodiments are not incompatible.

In some embodiments, the heterobifunctional compound of Formula (III) has the structure of Formula (V), or a pharmaceutically acceptable salt thereof, as described above. In some such embodiments, the compound of Formula (V) has the structure of Formula (V-A), (V-B), (V-C) or (V-D).

In some such embodiments, the compound of Formula (V) has the structure of Formula (V-A), wherein: X³ is N or CR^{1B}; X⁴ is N or CR^{1C}; and X⁶ is N or CR^{1E}. In some such embodiments, X³ is N; X⁴ is CR^{1C}; and X⁶ is CR^{1E}. In some such embodiments, X³ is CR^{1B}; X⁴ is CR^{1C}; and X⁶ is CR^{1E}. In some of the foregoing embodiments, R^{1E} is H.

In some such embodiments, the compound of Formula (V) has the structure of Formula (V-B), wherein: X⁵ is N or CR^{1D}; and X⁶ is N or CR^{1E}. In some such embodiments, X⁵ is CR^{1D}; and X⁶ is CR^{1E}. In some such embodiments, X⁵ is N; and X⁶ is CR^{1E}. In some such embodiments, X⁵ is CR^{1D}; and X⁶ is N. In some of the foregoing embodiments, R^{1D} is H. In some of the foregoing embodiments, R^{1E} is H.

In some such embodiments, the compound of Formula (V) has the structure of Formula (V-C), wherein: X⁵ is N or CR^{1D}. In some such embodiments, X⁵ is N. In some such embodiments, X⁵ is CR^{1D}.

In some such embodiments, the compound of Formula (V) has the structure of Formula (V-D).

In some embodiments, the compound of Formula (V) has the structure of any of Formula (V-a1) to (V-c2). In some such embodiments, the compound of Formula (V) has the structure of Formula (V-a1) or (V-a2).

In some embodiments, the heterobifunctional compound of Formula (III) has the structure of Formula (VI), or a pharmaceutically acceptable salt thereof, as described above.

In some such embodiments, the heterobifunctional compound of Formula (VI) has the structure of any of (VI-A) to (VI-N). In some such embodiments, the heterobifunctional compound of Formula (VI) has the structure of any of (VI-a1) to (VI-n3).

In some embodiments, the heterobifunctional compound of Formula (IV) has the structure of Formula (VII), or a pharmaceutically acceptable salt thereof, as described above.

In some such embodiments, the compound of Formula (VII) has the structure of Formula (VII-A), (VII-B), (VII-C) or (VII-D).

In some such embodiments, the compound of Formula (VII) has the structure of Formula (VII-A), wherein: X³ is N or CR^{1B}; X⁴ is N or CR^{1C}; and X⁶ is N or CR^{1E}. In some such embodiments, X³ is N; X⁴ is CR^{1C}; and X⁶ is CR^{1E}, where R^{1E} is H. In some such embodiments, X³ is CR^{1B}; X⁴ is CR^{1C}; and X⁶ is CR^{1E}, where R^{1E} is H.

In some such embodiments, the compound of Formula (VII) has the structure of Formula (VII-B), wherein: X⁵ is N or CR^{1D}; and X⁶ is N or CR^{1E}. In some such embodiments, X⁵ is CR^{1D}; and X⁶ is CR^{1E}. In some such embodiments, X⁵ is N; and X⁶ is CR^{1E}. In some such embodiments, X⁵ is CR^{1D}; and X¹ is N.

In some such embodiments, the compound of Formula (VII) has the structure of Formula (VII-C), wherein: X⁵ is N or CR^{1D}. In some such embodiments, X⁵ is N. In some such embodiments, X⁵ is CR^{1D}.

In some such embodiments, the compound of Formula (VII) has the structure of Formula (VII-D).

In some embodiments, the compound of Formula (VII) has the structure of any of Formula (VII-a1) to (VII-c2). In some such embodiments, the heterobifunctional compound of Formula (VII) has the structure of Formula (VII-a1) or (VII-a2).

In some embodiments, the heterobifunctional compound of Formula (IV) has the structure of Formula (VIII), or a pharmaceutically acceptable salt thereof, as described above.

In some such embodiments, the compound of Formula (VIII) has the structure of any of Formula (VIII-A) to (VIII-N). In some such embodiments, the compound of Formula (VIII) has the structure of any of Formula (VIII-a1) to (VIII-n3).

In preferred embodiments of compounds of any of Formulae (I)-(VIII), R² is H.

In some embodiments, the heterobifunctional compound of Formula (IV) has the structure of Formula (IX), or a pharmaceutically acceptable salt thereof, as described above.

In some such embodiments, the compound of Formula (IX) has the structure of any of Formula (IX-A) to (IX-N).

In some embodiments, the heterobifunctional compound of Formula (IV) has the structure of Formula (X) selected from any of Formulae (X-A) to (X-L), or a pharmaceutically acceptable salt thereof, as described above.

In some embodiments of any of Formulae (I)-(X), A is a PBM of any of Formulae (A), (A1), (A2), (A3) or (A4), as described herein. In some such embodiments, A is a PBM of any of Formulae (A5), (A), (A7), (A8) or (A9), as described herein. In some preferred embodiments, A is a PBM of Formula (A5).

In some embodiments, the heterobifunctional compound of Formula (IV) has the structure of Formula (XI), or a pharmaceutically acceptable salt thereof, as described above. In some such embodiments, the compound of Formula (XI) has the structure of Formula (XI-A), (XI0B), (XI-C) or (XI-D).

In some embodiments, the heterobifunctional compound of Formula (IV) has the structure of Formula (XII), or a pharmaceutically acceptable salt thereof, as described above. In some such embodiments, the compound of Formula (XII) has the structure of Formula (XII-A), (XII-B), (XII-C) or (XII-D).

Some preferred embodiments of the compounds or salts of any of Formulae (I)-(XII) have any combination of one, two, three, four, five, or more than five of the following selections, to the extent such embodiments are not incompatible:
A is a PBM capable of binding to CDK4 and/or CDK6;
A is a PBM having the structure of Formula (A);
A is a PBM having the structure of Formula (A1), (A2), (A3) or (A4);
A is a PBM having the structure of Formula (A5), (A6), (A7), (A8) or (A9);
A is a PBM having the structure of Formula (A5);
L¹ is a bond or a bivalent linker of Formula (L-1);
L¹ is a bond or a bivalent linker of Formula (L-1), wherein A_{L} is a bivalent moiety selected from the group consisting of R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b} and R_{L}^{a}N(R_{L}¹)R_{L}^{b};
L¹ is a bond or a bivalent linker of Formula (L-1), wherein A_{L} is a bivalent moiety selected from the group consisting of R_{L}^{a}C(O)R_{L}^{b}, R_{L}^{a}C(O)N(R_{L}¹)R_{L}^{b}, R_{L}^{a}N(R_{L}¹)C(O)R_{L}^{b}, and R_{L}^{a}N(R_{L}¹)R_{L}^{b};
L¹ is a bond or a bivalent linker of Formula (L-1), wherein B_{L} is a bivalent moiety selected from the group consisting of a bond, R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}²OR_{L}^{b} and R_{L}^{a}N(R_{L}¹)R_{L}^{b};
L¹ is a bond or a bivalent linker of Formula (L-1), wherein B_{L} is a bivalent moiety selected from the group consisting of a bond, R_{L}^{a}, R_{L}^{a}-R_{L}^{b}, R_{L}^{a}OR_{L}^{b} and R_{L}^{a}N(R_{L}¹)R_{L}^{b};
L¹ is a bond or a bivalent linker of Formula (L-1), wherein B_{L} is a bivalent moiety selected from the group consisting of a bond and R_{L}^{a}, where R_{L}^{a} is R_{L}^{r};
L¹ comprises one or more R_{L}^{r} moieties selected from the group consisting of Formula (L-2), Formula (L-3), Formula (L-4), Formula (L-5) and Formula (L-6);
R_{L}^{r} is selected from the group consisting of:

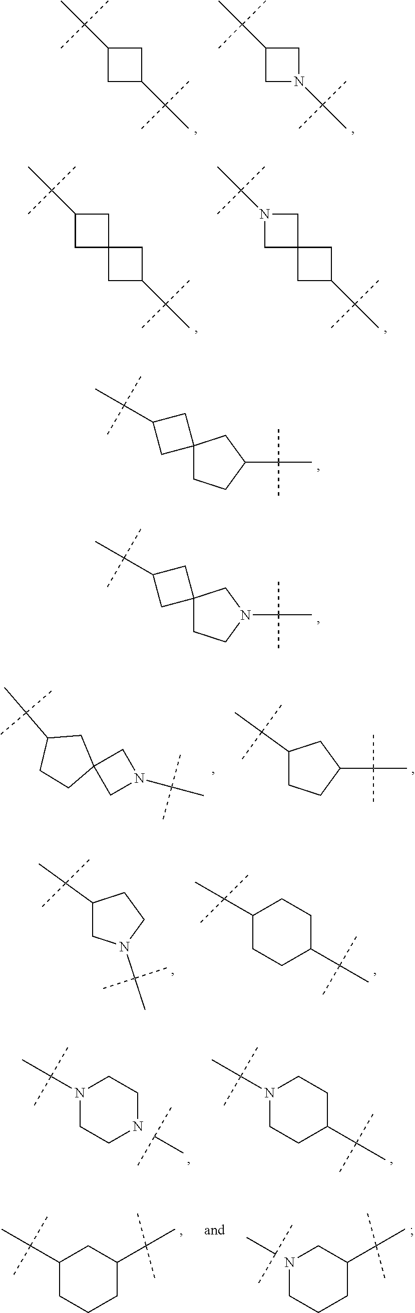

L¹ is a bond or a bivalent linker of Formula (J);
L² is —NH—;
Q is a 5-6 membered heteroaryl;
Q is a 5-6 membered heteroaryl selected from the group consisting of pyridine, pyridazine, imidazole, isoxazole, oxazole and pyrazole;
Q is pyridine or pyridazine;
Q is pyridin-2-yl or pyridazin-3-yl;
Q is imidazole, isoxazole, oxazole or pyrazole;
Q is imidazole;
Q is isoxazole;
Q is oxazole;
Q is pyrazole;
Q is selected from:

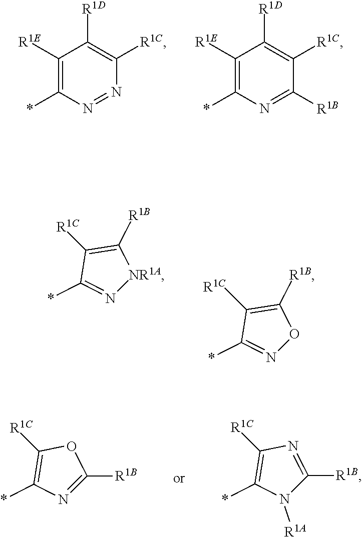

wherein * represents the point of attachment;
Q is selected from:

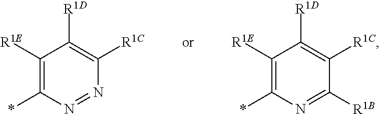

wherein * represents the point of attachment;
Q is selected from:

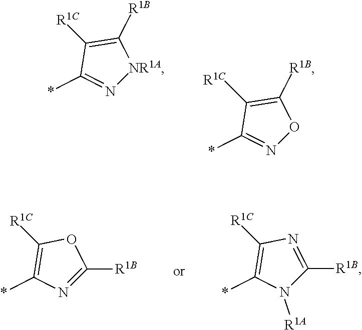

wherein * represents the point of attachment;
each R^{1A} is independently H, C₁-C₄ alkyl, or C₃-C₆ cycloalkyl;
each R^{1A} is independently H, CH₃, CH₂CH₃, CH₂CH₂CH₃, CH(CH₃)₂, cyclopropyl, or cyclobutyl;
each R^{1A} is independently H or C₁-C₄ alkyl;
each R^{1A} is independently H, CH₃, CH₂CH₃, CH₂CH₂CH₃ or CH(CH₃)₂;
each R^{1B}, R^{1C}, R^{1D} and R^{1E} is independently H, C₁-C₄ alkyl, C₁-C₄ alkoxy, halo, CN, NR^{5A}R^{5B}, C₃-C₆ cycloalkyl, or C₁-C₄ cycloalkoxy;
each R^{1B}, R^{1C}, R^{1D} and R^{1E} is independently H, CH₃, CH₂CH₃, CH₂CH₂CH₃, CH(CH₃)₂, OCH₃, F, Cl, NH₂, NHCH₃, N(CH₃)₂, N-pyrrolidinyl or cyclopropyl;
at least one of R^{1B}, R^{1C}, R^{1D} and R^{1E} is NR^{5A}R^{5B}, where each R^{5A} and R^{5B} is independently H, C₁-C₄ alkyl or C₃-C₆ cycloalkyl, or R^{5A} and R^{5B} are taken together with the N-atom to which they are attached to form a 4-6 membered heterocyclyl ring;
R² is H;
each R^{3A}, R^{3B} and R^{3C} is independently H, C₁-C₄ alkyl, C₁-C₄ alkoxy, halo, CN, NR^{7A}R^{7B} or C₃-C₆ cycloalkyl;
each R^{3A}, R^{3B} and R^{3C} is independently CH₃, CH₂CH₃, CH₂CH₂CH₃, CH(CH₃)₂, OCH₃, F, Cl, CN, NH₂, NHCH₃, NHCD₃, NHCH(CH₃)₂, NH(c-butyl), NH(t-butyl), N(CH₃)₂, N-pyrrolidinyl, or cyclopropyl;
at least one of R^{3A}, R^{3B} and R^{3C} is NR^{7A}R^{7B};
R^{3A} is NR^{7A}R^{7B};
each R^{4A} is independently C₁-C₄ alkoxy, OH, D, F, NH₂, NH(C₁-C₄-alkyl) or N(C₁-C₄-alkyl)₂;
each R^{4B} is independently C₁-C₄ alkoxy, OH, D, F, NH₂, NH(C₁-C₄-alkyl) or N(C₁-C₄-alkyl)₂;
each R^{4C} is independently C₁-C₄ alkyl, C₁-C₄ alkoxy, OH, D, F, NH₂, NH(C₁-C₄-alkyl) or N(C₁-C₄-alkyl)₂;
each R^{5A} and R^{5B} is independently H, C₁-C₄ alkyl or C₃-C₆ cycloalkyl, or R^{5A} and R^{5B} are taken together with the N-atom to which they are attached to form a 4-6 membered heterocyclyl ring;
each R^{6B} is independently C₁-C₄ alkoxy, OH, D, F, NH₂, NH(C₁-C₄-alkyl) or N(C₁-C₄-alkyl)₂;
each R^{6C} is independently C₁-C₄ alkyl, C₁-C₄ alkoxy, OH, D, F, NH₂, NH(C₁-C₄-alkyl) or N(C₁-C₄-alkyl)₂;
each R^{7A} and R^{7B} is independently H, C₁-C₄ alkyl or C₃-C₆ cycloalkyl, or R^{7A} and R^{7B} are taken together with the N-atom to which they are attached to form a 4-6 membered heterocyclyl ring;
each R^{8A} and R^{8B} is independently H, C₁-C₄ alkyl or C₃-C₆ cycloalkyl, or R^{8A} and R^{8B} are taken together with the N-atom to which they are attached to form a 4-6 membered heterocyclyl ring;
each R^{9A} and R^{9B} is independently H, C₁-C₄ alkyl or C₃-C₆ cycloalkyl, or R^{9A} and R^{9B} are taken together with the N-atom to which they are attached to form a 4-6 membered heterocyclyl ring;
each R^{10A} is independently C₁-C₄ alkoxy, OH, D, F, NH₂, NH(C₁-C₄-alkyl) or N(C₁-C₄-alkyl)₂;
each R^{10B} and R^{10C} is independently C₁-C₄ alkyl, C₁-C₄ alkoxy, F, Cl, CN, NH₂, NH(C₁-C₄-alkyl) or N(C₁-C₄-alkyl)₂;
each R^{11A}, R^{11B}, R^{12A} and R^{12B} is independently H, C₁-C₄ alkyl, or C₃-C₆ cycloalkyl;
each R^{11A}, R^{11B}, R^{12A} and R^{12B} is independently H or C₁-C₄ alkyl;
p is an integer from 1 to 3;
p is an integer from 1 to 2;
q is an integer from 1 to 2; and
q is 1.

Representative examples of heterobifunctional compounds of Formulae (I)-(XII) are shown in Table 4A and Table 4B.

### DDB1 Binding Moieties

Further described herein are monofunctional compounds comprising a DDB1 E3 ligase binding moiety. In some embodiments, such compounds may consist of the DDB1 binding moiety. In some embodiments, such compounds may consist essentially of the DDB1 binding moiety (e.g., a DDB1 binding moiety modified with a linker moiety, optionally including a reactive functional group, but lacking a PBM). Monofunctional compounds maybe useful for various purposes, e.g., as molecular glues or as synthetic intermediates for the preparation of heterobifunctional compounds, as further described herein.

Enumerated embodiments E50 to E61 relate to monofunctional compounds of Formula (XIII), and salts thereof, as well as compositions of such compounds and salts.

E50. A compound of Formula (XIII):

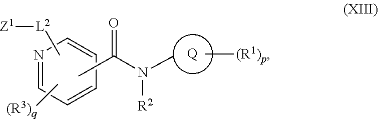

or a salt thereof, wherein:
Z¹ is L¹-Z² or L¹-G;
L¹ is a bond or a bivalent linker;
Z² is H, D, halo, C₁-C₄ alkyl, C₃-C₁₀ cycloalkyl, or 4-10 membered heterocyclyl, where each said C₁-C₄ alkyl, C₃-C₁₀ cycloalkyl, or 4-10 membered heterocyclyl is optionally substituted by halo, OH, oxo, C₁-C₄ alkoxy, NH₂, NH(C₁-C₄-alkyl), N(C₁-C₄-alkyl)₂;
G is a reactive functional group;
L² is a bond, —NH—, —N(C₁-C₃ alkyl)-, —NHC(O)—, —N(C₁-C₃ alkyl)C(O)—, —C(O)NH—, —C(O)N(C₁-C₃ alkyl)-, —O—, —C₁-C₄-alkylene-, —C₂-C₄-alkenylene- or —C₂-C₄-alkynylene-;
Q is C₆-C₁₀ aryl, 5-10 membered heteroaryl, C₃-C₁₀ cycloalkyl or 4-10 membered heterocyclyl;
each R¹ is independently R^{1A}, R^{1B}, R^{1C}, R^{1D} or R^{1E};
each R^{1A} is independently H, C₁-C₆ alkyl, C₃-C₆ cycloalkyl, or C₁-C₆ heteroalkyl, where each said C₁-C₆ alkyl, C₃-C₆ cycloalkyl, or C₁-C₆ heteroalkyl is optionally substituted by one or more R^{4A};
each R^{1B}, R^{1C}, R^{1D} and R^{1E} is independently H, C₁-C₆ alkyl, C₁-C₆ heteroalkyl, C₁-C₆ alkoxy, OH, D, halo, CN, NO₂, NR^{5A}R_{5B}, C(═O)R^{5A}, C(═O)OR^{5A}, OC(═O)R^{5A}, C(O)NR^{5A}R^{5B}, N(R^{5A})C(O)R^{5B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl, or C₁-C₆ alkoxy is optionally substituted by one or more R^{4B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{4C};
R² is H or C₁-C₃ alkyl;
each R³ is independently R^{3A}, R^{3B} or R^{3C}; or two R³ may be taken together with the atoms to which they are attached to form a C₃-C₁₀ cycloalkyl, 4-10 membered heterocyclyl, C₆-C₁₀ aryl or 5-10 membered heteroaryl, where each said C₃-C₁₀ cycloalkyl, 4-10 membered heterocyclyl, C₆-C₁₀ aryl or 5-10 membered heteroaryl is optionally substituted by one or more R^{A};
each R^{3A}, R^{3B} and R^{3C} is independently C₁-C₆ alkyl, C₁-C₆ heteroalkyl, C₁-C₆ alkoxy, OH, D, halo, CN, NO₂, NR^{7A}R^{7B}, C(═O)R^{7A}, C(═O)OR^{7A}, OC(═O)R^{7A}, C(O)NR^{7A}R^{7B}, N(R^{7A})C(O)R^{7B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{6B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{6C};
each R^{4A}, R^{4B} and R^{6B} is independently C₁-C₆ alkoxy, oxo, OH, D, halo, CN or NR^{8A}R^{8B};
each R^{4C}, R^{6A} and R^{6C} is independently C₁-C₄ alkyl, C₁-C₆ alkoxy, oxo, OH, D, halo, CN or NR^{9A}R^{9B};
each R^{5A}, R^{5B}, R^{7A}, R^{7B}, R^{8A}, R^{8B}, R^{9A} and R^{1B} is independently H, C₁-C₆ alkyl, C₁-C₆ heteroalkyl, C₃-C₁₀ cycloalkyl, 4-10 membered heterocyclyl, C₆-C₁₀ aryl or 5-10 membered heteroaryl, where each said C₁-C₆ alkyl or C₁-C₆ heteroalkyl is optionally substituted by one or more R^{10A}, and each said C₃-C₁O cycloalkyl, 4-10 membered heterocyclyl, C₆-C₁₀ aryl or 5-10 membered heteroaryl is optionally substituted by one or more R^{10B}; or
R^{5A} and R^{5B}, R^{7A} and R^{7B}, R^{5A} and R^{1B}, or R^{9A} and R^{1B} may be taken together with the N-atom to which they are attached to form a 4-10 membered heterocyclyl ring or 5-10 membered heteroaryl ring, where each said 4-10 membered heterocyclyl or 5-10 membered heteroaryl is optionally substituted by one or more R^{10C};
each R^{1A} is independently C₁-C₆ alkoxy, oxo, OH, D, halo, CN or NR^{11A}R^{11B};
each R^{10B} and R^{10C} is independently C₁-C₄ alkyl, C₁-C₆ alkoxy, oxo, OH, D, halo, CN or NR^{12A}R^{12B};
each R^{11A}, R^{11B}, R^{12A} and R^{12B} is independently H, C₁-C₄ alkyl, or C₃-C₆ cycloalkyl;
p is an integer from 0 to 4; and
q is an integer from 0 to 3;
with the proviso the compound of Formula (XIII) is not:
2-amino-N-(4,5-dimethylthiazol-2-yl)-6-methylnicotinamide;
tert-butyl (2-(2-(3-((3-((4,5-dimethylthiazol-2-yl)carbamoyl)-6-methylpyridin-2-yl)amino)-3-oxopropoxy)ethoxy)ethyl)carbamate;
2-(3-(2-(2-aminoethoxy)ethoxy)propanamido)-N-(4,5-dimethylthiazol-2-yl)-6-methylnicotinamide;
tert-butyl (7-((6-chloro-3-((4,5-dimethylthiazol-2-yl)carbamoyl)-pyridin-2-yl)amino)heptyl)carbamate;
tert-butyl (7-((3-((4,5-dimethylthiazol-2-yl)carbamoyl)-6-methylpyridin-2-yl)amino)heptyl)carbamate; or
2-((7-aminoheptyl)amino)-N-(4,5-dimethylthiazol-2-yl)-6-methylnicotinamide.
   E51. The compound of embodiment E50, having the structure of Formula (XIII-A), (XIII-B), (XIII-C) or (XIII-D):

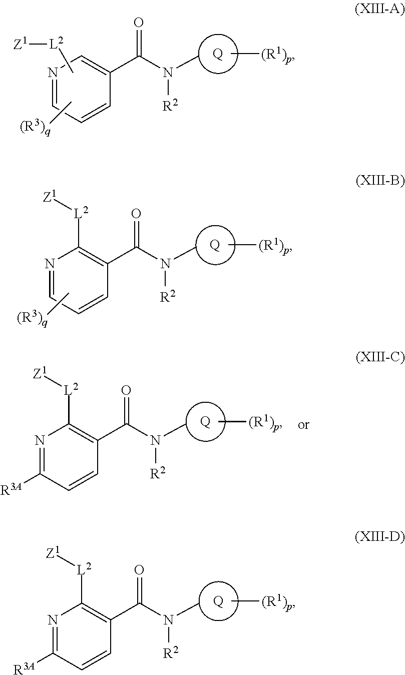

or a salt thereof.
   E52. The compound or salt of embodiment E50 or E51, wherein Q is phenyl or 5-6 membered heteroaryl.
   E53. The compound or salt of embodiment E52, wherein Q is selected from the group consisting of pyridine, pyridazine, imidazole, isoxazole, oxazole and pyrazole.
   E54. The compound or salt of embodiment E53, wherein Q is selected from:

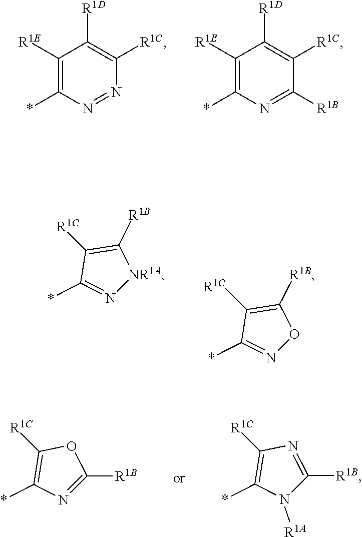

wherein * represents the point of attachment.
   E55. The compound or salt of embodiment E54, wherein:
each R^{1A} is independently H, C₁-C₄ alkyl, or C₃-C₆ cycloalkyl; and
each R^{1B}, R^{1C}, R^{1D} and R^{1E} is independently H, C₁-C₄ alkyl, C₁-C₄ alkoxy, halo, CN, NR^{5A}R^{5B} C₃-C₆ cycloalkyl, or C₃-C_{E} cycloalkoxy.
   E56. The compound or salt of any one of embodiments E50 to E55, Z¹ is L¹-G.
   E57. The compound or salt of any one of embodiments E50 to E56, wherein G is selected from the group consisting of protected or unprotected primary or secondary amine, carboxylic acid, carboxylate ester, halo, hydroxy, C₁-C₄ alkoxy, C₁-C₄ haloalkoxy, formyl, C₁-C₄ alkyl sulfonate ester, C₁-C₄ haloalkyl sulfonate ester, C₆-C₁₀ aryl sulfonate ester, boronic acid, boronate ester, and azide.
   E58. The compound or salt of any one of embodiments E50 to E57, wherein, G is halo, OH, OR^{d}, NH₂, NH(C₁-C₄-alkyl), NH—CO₂R^{d}, N₃, CO₂H, CO₂R^{d} or OSO₂R^{d}, where each R^{d} is independently C₁-C₄ alkyl, C₁-C₄ haloalkyl, C₂-C₄ alkenyl, optionally substituted C₇-C₁₄ arylalkyl, or optionally substituted C₆-C₁₃ aryl.
   E59. The compound or salt of any one of embodiments E50 to E55, wherein Z¹ is L¹-Z².
   E60. The compound or salt of embodiment E59, wherein Z² is H, D, or halo.
   E61. The compound or salt of embodiment E59, wherein Z² is C₁-C₄ alkyl, C₃-C₁₀ cycloalkyl, or 4-10 membered heterocyclyl, where each said C₁-C₄ alkyl, C₃-C₁₀ cycloalkyl, or 4-10 membered heterocyclyl is optionally substituted by halo, OH, oxo, C₁-C₄ alkoxy, NH₂, NH(C₁-C₄-alkyl), N(C₁-C₄-alkyl)₂.

The embodiments described herein for compounds of any of Formulae (I)-(XII) with respect to Q, R¹, R², R³, R^{3A}, L¹, L², p and q are also applicable to compounds or salts of Formula (XIII), (XIII-A), (XIII-B), (XIII-C) or (XIII-D), to the extent such embodiments are not incompatible.

In some embodiments of Formula (XIII), L¹ is a bond. In some embodiments of Formula (XIII), L¹ is a bivalent linker of Formula (L), as described herein. In some preferred embodiments of Formula (XIII), L¹ is a bivalent linker of Formula (L-1), as described herein. In some embodiments of Formula (XIII), L¹ is a bivalent linker of Formula (J), as described herein.

In some embodiments of Formula (XIII), (XIII-A), (XIII-B), (XIII-C) or (XIII-D), G is selected from the group consisting of protected or unprotected primary or secondary amine, carboxylic acid, carboxylate ester (e.g., C₁-C₄ alkyl, C₂-C₄ alkenyl, benzyl or PMB ester), halo, hydroxy, C₁-C₄ alkoxy, C₁-C₄ haloalkoxy, formyl, C₁-C₄ alkyl sulfonate ester (e.g., mesylate), C₁-C₄ haloalkyl sulfonate ester (e.g., triflate), C₆-C₁₀ aryl sulfonate ester (e.g., tosylate), boronic acid, boronate ester, and azide. In some embodiments, G is a carbamate-protected primary amine (e.g., NH-Boc, NH-Cbz, NH-Fmoc, or NH-Alloc), an unprotected primary amine, a carboxylic acid, a halo, or a hydroxy moiety.

Some preferred embodiments of Formula (XIII), (XIII-A), (XIII-B), (XIII-C) or (XIII-D) have any combination of one, two, three, four, five, or more than five of the following selections to the extent such embodiments are not incompatible:
L¹ is a bond or a bivalent linker of Formula (L-1);
L¹ is a bond or a bivalent linker of Formula (L-1), wherein A_{L} is a bivalent moiety selected from the group consisting of R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b}, and R_{L}^{a}N(R_{L}¹)R_{L}^{b};
L¹ is a bond or a bivalent linker of Formula (L-1), wherein B_{L} is a bivalent moiety selected from the group consisting of a bond, R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b}, and R_{L}^{a}N(R_{L}¹)R_{L}^{b};
L¹ is a bond or a bivalent linker of Formula (J);
L² is —NH—;
G is halo, OH, OR^{d}, NH₂, NH(C₁-C₄-alkyl), NH—CO₂R^{d}, N₃, CO₂H, CO₂R^{d} or OSO₂R^{d}, where each R^{d} is independently C₁-C₄ alkyl, C₁-C₄ haloalkyl, C₂-C₄ alkenyl, optionally substituted C₇-C₁₄ arylalkyl, or optionally substituted C₆-C₁₃ aryl;
Q is a 5-6 membered heteroaryl;
Q is a 5-6 membered heteroaryl selected from the group consisting of pyridine, pyridazine, imidazole, isoxazole, oxazole and pyrazole;
Q is pyridine or pyridazine;
Q is pyridin-2-yl or pyridazin-3-yl;
Q is imidazole, isoxazole, oxazole or pyrazole;
Q is imidazole;
Q is isoxazole;
Q is oxazole;
Q is pyrazole;
Q is selected from:

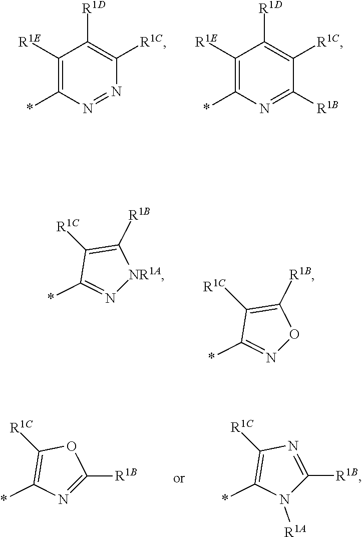

wherein * represents the point of attachment;
Q is selected from:

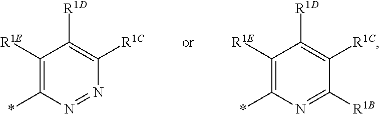

wherein * represents the point of attachment;
Q is selected from:

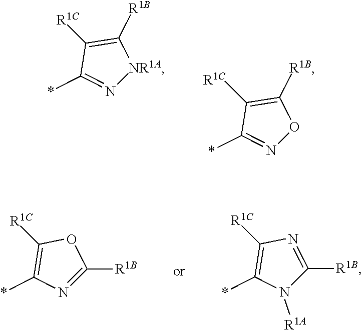

wherein * represents the point of attachment;
each R^{1A} is independently H, C₁-C₄ alkyl, or C₃-C₆ cycloalkyl;
each R^{1A} is independently H, CH₃, CH₂CH₃, CH₂CH₂CH₃, CH(CH₃)₂, cyclopropyl, or cyclobutyl;
each R^{1A} is independently H or C₁-C₄ alkyl;
each R^{1A} is independently H, CH₃, CH₂CH₃, CH₂CH₂CH₃ or CH(CH₃)₂;
each R^{1B}, R^{1C}, R^{1D} and R^{1E} is independently H, C₁-C₄ alkyl, C₁-C₄ alkoxy, halo, CN, NR^{5A}R^{5B}, C₃-C₆ cycloalkyl, or C₃-C₆ cycloalkoxy;
each R^{1B}, R^{1C}, R^{1D} and R^{1E} is independently H, CH₃, CH₂CH₃, CH₂CH₂CH₃, CH(CH₃)₂, OCH₃, F, Cl, NH₂, NHCH₃, N(CH₃)₂, N-pyrrolidinyl or cyclopropyl;
at least one of R^{1B}, R^{1C}, R^{1D} and R^{1E} is NR^{5A}R^{5B}, where each R^{1A} and R^{5B} is independently H, C₁-C₄ alkyl or C₃-C₆ cycloalkyl, or R^{5A} and R^{5B} are taken together with the N-atom to which they are attached to form a 4-6 membered heterocyclyl ring;
R² is H;
each R^{3A}, R^{3B} and R^{3C} is independently H, C₁-C₄ alkyl, C₁-C₄ alkoxy, halo, CN, NR^{7A}R^{7B} or C₃-C₆ cycloalkyl;
each R^{3A}, R^{3B} and R^{3C} is independently CH₃, CH₂CH₃, CH₂CH₂CH₃, CH(CH₃)₂, OCH₃, F, Cl, CN, NH₂, NHCH₃, NHCD₃, NHCH(CH₃)₂, NH(c-butyl), NH(t-butyl), N(CH₃)₂, N-pyrrolidinyl, or cyclopropyl;
at least one of R^{3A}, R^{3B} and R^{3C} is NR^{7A}R^{7B};
R^{3A} is NR^{7A}R^{7B};
each R^{4A} is independently C₁-C₄ alkoxy, OH, D, F, NH₂, NH(C₁-C₄-alkyl) or N(C₁-C₄-alkyl)₂;
each R^{4B} is independently C₁-C₄ alkoxy, OH, D, F, NH₂, NH(C₁-C₄-alkyl) or N(C₁-C₄-alkyl)₂;
each R^{4C} is independently C₁-C₄ alkyl, C₁-C₄ alkoxy, OH, D, F, NH₂, NH(C₁-C₄-alkyl) or N(C₁-C₄-alkyl)₂;
each R^{5A} and R^{5B} is independently H, C₁-C₄ alkyl or C₃-C₆ cycloalkyl, or R^{5A} and R^{5B} are taken together with the N-atom to which they are attached to form a 4-6 membered heterocyclyl ring;
each R^{6B} is independently C₁-C₄ alkoxy, OH, D, F, NH₂, NH(C₁-C₄-alkyl) or N(C₁-C₄-alkyl)₂;
each R^{6C} is independently C₁-C₄ alkyl, C₁-C₄ alkoxy, OH, D, F, NH₂, NH(C₁-C₄-alkyl) or N(C₁-C₄-alkyl)₂;
each R^{7A} and R^{7B} is independently H, C₁-C₄ alkyl or C₃-C₆ cycloalkyl, or R^{7A} and R^{7B} are taken together with the N-atom to which they are attached to form a 4-6 membered heterocyclyl ring;
each R^{8A} and R^{8B} is independently H, C₁-C₄ alkyl or C₃-C₆ cycloalkyl, or R^{8A} and R^{8B} are taken together with the N-atom to which they are attached to form a 4-6 membered heterocyclyl ring;
each R^{9A} and R^{9B} is independently H, C₁-C₄ alkyl or C₃-C₆ cycloalkyl, or R^{9A} and R^{9B} are taken together with the N-atom to which they are attached to form a 4-6 membered heterocyclyl ring;
each R^{10A} is independently C₁-C₄ alkoxy, OH, D, F, NH₂, NH(C₁-C₄-alkyl) or N(C₁-C₄-alkyl)₂;
each R^{10B} and R^{10C} is independently C₁-C₄ alkyl, C₁-C₄ alkoxy, F, Cl, CN, NH₂, NH(C₁-C₄-alkyl) or N(C₁-C₄-alkyl)₂;
each R^{11A}, R^{11B}, R^{12A} and R^{12B} is independently H, C₁-C₄ alkyl, or C₃-C₆ cycloalkyl;
each R^{11A}, R^{11B}, R^{12A} and R^{12B} is independently H or C₁-C₄ alkyl;
p is an integer from 1 to 3;
p is an integer from 1 to 2;
q is an integer from 1 to 2; and
q is 1.

Representative compounds of Formula (XIII) are shown in Tables 1A and 1B.

Linkers Described herein are compounds comprising a bivalent linker. In some embodiments, provided herein are compounds of any of Formulae (I)-(XIII), comprising a DDB1 binding moiety and a linker moiety, L¹, which may be a bond or a bivalent linker. In some embodiments of any of Formulae (I)-(XI), L¹ is covalently attached to a PBM. In some embodiments of any of Formulae (I)-(XIII), L¹ is covalently attached to the DDB1 binding moiety via L². In some embodiments of any of Formulae (I)-(XII), L¹ is covalently attached to both a PBM and the DDB1 binding moiety via L².

In some embodiments of any of Formulae (I)-(XIII), L¹ is a bivalent linker of Formula (L):

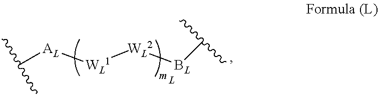

wherein:
each A_{L}, W_{L}¹, W_{L}² and B_{L} is a bivalent moiety independently selected from the group consisting of a bond, R_{L}^{a}, R_{L}^{a}-R_{L}^{b}, R_{L}^{a}C(O)R_{L}^{b}, R_{L}^{a}C(O)OR_{L}^{b}, R_{L}^{a}OC(O)R_{L}^{b}, R_{L}^{a}C(O)N(R_{L}¹)R_{L}^{b}, R_{L}^{a}N(R_{L}¹)C(O)R_{L}^{b}, R_{L}^{a}C(S)N(R_{L}¹)R_{L}^{b}, R_{L}^{a}N(R_{L}¹)C(S)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)SO₂R_{L}^{b}, R_{L}^{a}N(R_{L}¹)R_{L}^{b}, and R_{L}^{a}N(R_{L}¹)C(O)N(R_{L}²)R_{L}^{b};
each R_{L}^{a} and R_{L}^{b} is independently selected from the group consisting of a bond, R_{L}^{r}, (C₁-C₈ alkylene)-R_{L}^{r}, R_{L}^{r}-(C₁-C₈ alkylene), (C₁-C₈ alkylene)-R_{L}^{r}-(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, where each said C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene is optionally substituted by one or more R_{L}^{c};
each R_{L}^{r} is independently selected from the group consisting of C₃-C₁₂ cycloalkylene, 3-12 membered heterocyclene, C₆-C₁₂ arylene, and 5-13 membered heteroarylene, where each said C₃-C₁₂ cycloalkylene or 3-12 membered heterocyclene is optionally substituted by one or more R_{L}^{d}, and each said C₆-C₁₂ arylene or 5-13 membered heteroarylene is optionally substituted by one or more R_{L}^{e};
each R_{L}¹ and R_{L}² is independently selected from the group consisting of H, C₁-C₈ alkyl, C₂-C₈ alkenyl, C₂-C₈ alkynyl, C₃-C₁₂ cycloalkyl, 3-12 membered heterocyclyl, C₆-C₁₂ aryl, and 5-13 membered heteroaryl, where each said C₁-C₈ alkyl, C₂-C₈ alkenyl or C₂-C₈ alkynyl is optionally substituted by one or more R_{L}^{f}, each said C₃-C₁₂ cycloalkyl or 3-12 membered heterocyclyl is optionally substituted by one or more R_{L}^{g}, and each said C₆-C₁₂ aryl or 5-13 membered heteroaryl is optionally substituted by one or more R_{L}^{h}; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₁₂ carbocyclyl or 3-12 membered heterocyclyl;
each R_{L}^{c} and R_{L}^{f} is independently selected from the group consisting of F, OH, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl);
each R_{L}^{d} and R_{L}^{g} is independently selected from the group consisting of F, OH, C₁-C₄ alkyl, C₁-C₄ fluoroalkyl, C₃-C₆ cycloalkyl, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl);
each R_{L}^{e} and R_{L}^{h} is independently selected from the group consisting of halo, OH, C₁-C₄ alkyl, C₁-C₄ fluoroalkyl, C₃-C₆ cycloalkyl, C₁-C₄ alkoxy, C₃-C₆ cycloalkoxy, oxo, CN, NH₂, NH(C₁-C₄ alkyl), N(C₁-C₄ alkyl)₂, NH(C₃-C₈ cycloalkyl), and N(C₁-C₄ alkyl)(C₃-C₆ cycloalkyl); or
two of R_{L}^{e}, R_{L}^{d}, R_{L}^{e}, R_{L}^{f}, R_{L}^{g}, and R_{L}^{h} together with the atom(s) to which they are attached optionally form a C₃-C₁₂ carbocyclyl or 3-12 membered heterocyclyl; and
m_{L} is an integer selected from 0 to 15.

In some embodiments of Formula (L), mL is 0, and W_{L}¹ and W_{L}² are null.

In some embodiments of Formula (L), mL is 0, and the bivalent linker has the structure of Formula (L-1), wherein A_{L} and B_{L} are defined as for Formula (L).

In some embodiments of any of Formulae (I)-(XIII), L¹ is a linker of Formula (L-1):

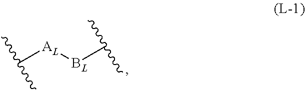

wherein:
each A_{L}, and B_{L} is a bivalent moiety independently selected from the group consisting of a bond, R_{L}^{a}, R_{L}^{a}-R_{L}^{b}, R_{L}^{a}C(O)R_{L}^{b}, R_{L}^{a}C(O)OR_{L}^{b}, R_{L}^{a}OC(O)R_{L}^{b}, R_{L}^{a}C(O)N(R_{L}¹)R_{L}^{b}, R_{L}^{a}N(R_{L}¹)C(O)R_{L}^{b}, R_{L}^{a}C(S)N(R_{L}¹)R_{L}^{b}, R_{L}^{a}N(R_{L}¹)C(S)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)SO₂R_{L}^{b}, R_{L}^{a}N(R_{L}¹)R_{L}^{b}, and R_{L}^{a}N(R_{L}¹)C(O)N(R_{L}²)R_{L}^{b};
each R_{L}^{a} and R_{L}^{b} is independently selected from the group consisting of a bond, R_{L}^{r}, (C₁-C₈ alkylene)-R_{L}^{r}, R_{L}^{r}-(C₁-C₈ alkylene), (C₁-C₈ alkylene)-R_{L}^{r}-(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, where each said C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene moiety is optionally substituted by one or more R_{L}^{c}; each R_{L}^{r} is independently selected from the group consisting of C₃-C₁₂ cycloalkylene, 3-12 membered heterocyclene, C₆-C₁₂ arylene, and 5-13 membered heteroarylene, where each said C₃-C₁₂ cycloalkylene or 3-12 membered heterocyclene is optionally substituted by one or more R_{L}^{d}, and each said C₆-C₁₂ arylene or 5-13 membered heteroarylene is optionally substituted by one or more R_{L}^{e}; each R_{L}¹ and R_{L}² is independently selected from the group consisting of H, C₁-C₈ alkyl, C₂-C₈ alkenyl, C₂-C₈ alkynyl, C₃-C₁₂ cycloalkyl, 3-12 membered heterocyclyl, C₆-C₁₂ aryl, and 5-13 membered heteroaryl, where each said C₁-C₈ alkyl, C₂-C₈ alkenyl or C₂-C₈ alkynyl is optionally substituted by one or more R_{L}^{f}, each said C₃-C₁₂ cycloalkyl or 3-12 membered heterocyclyl is optionally substituted by one or more R_{L}^{g}, and each said C₆-C₁₂ aryl or 5-13 membered heteroaryl is optionally substituted by one or more R_{L}^{h}; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₁₂ carbocyclyl or 3-12 membered heterocyclyl;
each R_{L}^{c} and R_{L}^{f} is independently selected from the group consisting of F, OH, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl);
each R_{L}^{d} and R_{L}^{g} is independently selected from the group consisting of F, OH, C₁-C₄ alkyl, C₁-C₄ fluoroalkyl, C₃-C₆ cycloalkyl, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); and
each R_{L}^{c} and R_{L}^{h} is independently selected from the group consisting of halo, OH, C₁-C₄ alkyl, C₁-C₄ fluoroalkyl, C₃-C₆ cycloalkyl, C₁-C₄ alkoxy, C₃-C₆ cycloalkoxy, oxo, CN, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); or
two R_{L}^{c}, R_{L}^{d}, R_{L}^{e}, R_{L}, R_{L}^{g}, and R_{L}^{h} together with the atom(s) to which they are attached optionally form a C₃-C₁₂ carbocyclyl or 3-12 membered heterocyclyl.

In some embodiments of Formula (L) or Formula (L-1), A_{L} is a bond. In some embodiments of Formula (L) or Formula (L-1), A_{L} is R_{L}^{a}, and R_{L}^{a} is C₁-C₈ alkylene. In some embodiments of Formula (L) or Formula (L-1), A_{L} is R_{L}^{a}N(R_{L}¹)C(O)R_{L}^{b}, R_{L}^{a} is C₁-C₈ alkylene and R_{L}^{b} is C₁-C₈ alkylene. In some embodiments of Formula (L) or Formula (L-1), A_{L} is R_{L}^{a}N(R_{L}¹)C(O)R_{L}^{b}, R_{L}^{a} is C₁-C₈ alkylene and R_{L}^{b} is a bond. In some embodiments of Formula (L) or Formula (L-1), A_{L} is R_{L}^{a}—N(R_{L}¹)C(O)R_{L}^{b}, R_{L}^{a} is a bond and R_{L}^{b} is C₁-C₈ alkylene. In some embodiments of Formula (L) or Formula (L-1), A_{L} is R_{L}^{a}N(R_{L}¹)C(O)R_{L}^{b}, R_{L}^{a} is a bond and R_{L}^{h} is a bond. In some embodiments of Formula (L) or Formula (L-1), A_{L} is R_{L}^{a}C(O)N(R_{L}¹)R_{L}^{b}, R_{L}^{a} is C₁-C₈ alkylene and R_{L}^{b} is C₁-C₈ alkylene. In some embodiments of Formula (L) or Formula (L-1), A_{L} is R_{L}^{a}C(O)N(R_{L}¹)R_{L}^{b}, R_{L}^{a} is C₁-C₈ alkylene and R_{L}^{b} is a bond. In some embodiments of Formula (L) or Formula (L-1), A_{L} is R_{L}^{a}C(O)N(R_{L}¹)R_{L}^{b}, R_{L}^{a} is a bond and R_{L}^{b} is C₁-C₈ alkylene. In some embodiments of Formula (L) or Formula (L-1), A_{L} is R_{L}^{a}C(O)N(R_{L}¹)R_{L}^{b}, R_{L}^{a} is a bond and R_{L}^{b} is a bond. In some embodiments of Formula (L) or Formula (L-1), A_{L} is R_{L}^{a}C(O)R_{L}^{b}, R_{L}^{a} is a bond and R_{L}^{b} is C₁-C₈ alkylene. In some embodiments of Formula (L) or Formula (L-1), A_{L} is R_{L}^{a}C(O)R_{L}^{b}, R^{a} is a C₁-C₈ alkylene and R_{L}^{b} is a bond. In some embodiments of Formula (L) or Formula (L-1), A_{L} is R_{L}^{a}C(O) R_{L}^{b}, R_{L}^{a} is a bond and R_{L}^{b} is a bond. In some embodiments of any of the foregoing, B_{L} is a bivalent moiety selected from the group consisting of: a bond; R_{L}^{a}, where R_{L}^{a} is R_{L}^{r} or R_{L}^{r}—(C₁-C₈ alkylene); R_{L}^{a}OR_{L}^{b}, where R_{L}^{a} is a bond and R_{L}^{b} is R_{L}^{r}, R_{L}R^{r}(C₁-C₈ alkylene) or (C₁-C₈ alkylene)-R_{L}^{r}; and R_{L}^{a}N(R_{L}¹)R_{L}^{b}, where R_{L}^{a} is a bond and R_{L}^{b} is R_{L}^{r}; wherein each R_{L}U moiety is optionally substituted C₃-C₁₂ cycloalkylene or 3-12 membered heterocyclene.

In some embodiments of Formula (L) or Formula (L-1), two R_{L}^{c} groups, together with the atom(s) to which they are attached optionally form a C₃-C₆ carbocyclyl or 3-6 membered heterocyclyl.

In some embodiments of any of Formulae (I)-(XIII), L is a bivalent linker of Formula (L) or Formula (L-1), wherein:
A_{L} is a bivalent moiety selected from the group consisting of:
   1) R_{L}^{a}C(O)N(R_{L}¹)R_{L}^{b}, where:
      (a) R_{L}^{a} is C₁-C₈ alkylene and R_{L}^{b} is C₁-C₈ alkylene;
      (b) R_{L}^{a} is C₁-C₈ alkylene and R_{L}^{b} is a bond;
      (c) R_{L}^{a} is a bond and R_{L}^{b} is C₁-C₈ alkylene;
      (d) R_{L}^{a} is a bond and R_{L}^{b} is a bond;
      (e) R_{L}^{a} is (C₁-C₈ alkylene)-R_{L}^{T} and R_{L}^{b} is C₁-C₈ alkylene;
      (i) R_{L}^{a} is C₁-C₈ alkylene and R_{L}^{b} is R_{L}^{r}-(C₁-C₈ alkylene);
      (g) R_{L}^{a} is C₁-C₈ alkylene and R_{L}^{b} is (C₁-C₈ alkylene)-R_{L}^{r}-(C₁-C₈ alkylene);
      (h) R_{L}^{a} is C₁-C₈ alkylene and R_{L}^{b} is (C₁-C₈ alkylene)-R_{L}^{r};
      (i) R_{L}^{a} is C₁-C₈ alkylene and R_{L}^{b} is R_{L};
      (j) R_{L}^{a} is a bond and R_{L}^{b} is (C₁-C₈ alkylene)-R_{L}^{r}-(C₁-C₈ alkylene);
      (k) R_{L}^{a} is a bond and R_{L}^{b} is R_{L}^{r}-(C₁-C₈ alkylene);
      (l) R_{L}^{a} is a bond and R_{L}^{b} is (C₁-C₈ alkylene)-R_{L}^{r}; or
      (m) R_{L}^{a} is a bond and R_{L}^{b} is R_{L}^{r};
   2) R_{L}^{a}N(R_{L}¹)C(O)R_{L}^{b}, where:
      (a) R_{L}^{a} is C₁-C₈ alkylene and R_{L}^{b} is C₁-C₈ alkylene;
      (b) R_{L}^{a} is C₁-C₈ alkylene and R_{L}^{b} is a bond;
      (c) R_{L}^{a} is a bond and R_{L}^{b} is C₁-C₈ alkylene;
      (d) R_{L}^{a} is a bond and R_{L}^{b} is a bond;
      (e) R_{L}^{a} is (C₁-C₈ alkylene)-R_{L}^{T} and R_{L}^{b} is C₁-C₈ alkylene;
      (t) R_{L}^{a} is C₁-C₈ alkylene and R_{L}^{b} is R_{L}^{T}—(C₁-C₈ alkylene);
      (g) R_{L}^{a} is C₁-C₈ alkylene and R_{L}^{b} is (C₁-C₈ alkylene)-R_{L}^{r}-(C₁-C₈ alkylene);
      (h) R_{L} is C₁-C₈ alkylene and R_{L}^{b} is (C₁-C₈ alkylene)-R_{L}^{r};
      (i) R_{L}^{a} is C₁-C₈ alkylene and R_{L}^{b} is R_{L}^{r};
      (j) R_{L}^{a} is a bond and R_{L}^{b} is (C₁-C₈ alkylene)-R_{L}^{T}—(C₁-C₈ alkylene);
      (k) R_{L}^{a} is a bond and R_{L}^{b} is R_{L}^{r}-(C₁-C₈ alkylene);
      (l) R_{L}^{a} is a bond and R_{L}^{b} is (C₁-C₈ alkylene)-R_{L}^{T}; or
      (m) R_{L}^{a} is a bond and R_{L}^{b} is R_{L}^{T};
   3) R_{L}^{a}C(O)R_{L}^{b}, where:
      (a) R_{L}^{a} is C₁-C₈ alkylene and R_{L}^{b} is C₁-C₈ alkylene;
      (b) R_{L}^{a} is C₁-C₈ alkylene and R_{L}^{b} is a bond;
      (c) R_{L}^{a} is a bond and R_{L}^{b} is C₁-C₈ alkylene;
      (d) R_{L}^{a} is a bond and R_{L}^{b} is a bond;
      (e) R_{L}^{a} is (C₁-C₈ alkylene)-R_{L}^{r} and R_{L}^{b} is C₁-C₈ alkylene;
      (f) R_{L}^{a} is C₁-C₈ alkylene and R_{L}^{b} is (C₁-C₈ alkylene)-R_{L}^{r}-(C₁-C₈ alkylene);
      (g) R_{L}^{a} is C₁-C₈ alkylene and R_{L}^{b} is (C₁-C₈ alkylene)-R_{L}^{r};
      (h) R_{L}^{a} is C₁-C₈ alkylene and R_{L}e is R_{L}^{r};
      (i) R_{L}^{a} is C₁-C₈ alkylene and R_{L}e is R_{L}^{r}-(C₁-C₈ alkylene);
      (j) R_{L}^{a} is a bond and R_{L}^{b} is (C₁-C₈ alkylene)-R_{L}^{r}-(C₁-C₈ alkylene);
      (k) R_{L}^{a} is a bond and R_{L}^{b} is R_{L}^{r}-(C₁-C₈ alkylene);
      (l) R_{L}^{a} is a bond and R_{L}^{b} is (C₁-C₈ alkylene)-R_{L}^{r}; or
      (m) R_{L}^{a} is a bond and R_{L}^{b} is R_{L}^{r};
   4) R_{L}^{a}N(R_{L}¹)R_{L}^{b}, where:
      (a) R_{L}^{a} is C₁-C₈ alkylene and R_{L}e is C₁-C₈ alkylene;
      (b) R_{L}^{a} is C₁-C₈ alkylene and R_{L}^{b} is a bond;
      (c) R_{L}^{a} is a bond and R_{L}^{b} is C₁-C₈ alkylene;
      (d) R_{L}^{a} is a bond and R_{L}^{b} is a bond;
      (e) R_{L}^{a} is (C₁-C₈ alkylene)-R_{L}^{r} and R_{L}^{b} is C₁-C₈ alkylene;
      (f) R_{L}^{a} is C₁-C₈ alkylene and R_{L}^{b} is (C₁-C₈ alkylene)-R_{L}^{r}-(C₁-C₈ alkylene);
      (g) R_{L}^{a} is C₁-C₈ alkylene and R_{L}^{b} is (C₁-C₈ alkylene)-R_{L}^{r};
      (h) R_{L}^{a} is C₁-C₈ alkylene and R_{L}^{b} is R_{L}^{r};
      (i) R_{L}^{a} is C₁-C₈ alkylene and R_{L}^{b} is R_{L}^{r}-(C₁-C₈ alkylene);
      (j) R_{L}^{a} is a bond and R_{L}^{b} is (C₁-C₈ alkylene)-R_{L}^{r}-(C₁-C₈ alkylene);
      (k) R_{L}^{a} is a bond and R_{L}^{b} is R_{L}^{r}-(C₁-C₈ alkylene);
      (l) R_{L}^{a} is a bond and R_{L}^{b} is (C₁-C₈ alkylene)-R_{L}^{r}; or
      (m) R_{L}^{a} is a bond and R_{L}^{b} is R_{L}^{r}; and
   5) R_{L}^{a}-R_{L}^{b}, where:
      (a) R_{L}^{a} is C₁-C₈ alkylene and R_{L}^{b} is a bond;
      (b) R_{L}^{a} is (C₁-C₈ alkylene)-R_{L}^{r} and R_{L}^{b} is C₁-C₈ alkylene;
      (c) R_{L}^{a} is (C₁-C₈ alkylene)-R_{L}^{T} and R_{L}^{b} is a bond; or
      (d) R_{L}^{a} is R_{L}^{r}-(C₁-C₈ alkylene) and R_{L}^{b} is a bond;
wherein each R_{L}¹ moiety is C₃-C₁₂ cycloalkylene or 3-12 membered heterocyclene;
each C₁-C₈ alkylene moiety is optionally substituted by one or more R_{L}^{c}; and
each C₃-C₁₂ cycloalkylene or 3-12 membered heterocyclene is optionally substituted by one or more R_{L}^{d}.

In some embodiments any of Formulae (I)-(XIII), two of R_{L}^{c} groups, together with the atom(s) to which they are attached optionally form a C₃-C₆ carbocyclyl or 3-6 membered heterocyclyl.

In some embodiments of any of Formulae (I)-(XIII), L¹ is a bivalent linker of Formula (L) or Formula (L-1), wherein:
A_{L} is R_{L}^{a}C(O)N(R_{L}¹)R_{L}^{b}, and:
   (a) R_{L}^{a} is C₁-C₄ alkylene and R_{L}^{b} is C₁-C₄ alkylene;
   (b) R_{L}^{a} is C₁-C₄ alkylene and R_{L}^{b} is a bond;
   (c) R_{L}^{a} is a bond and R_{L}^{b} is C₁-C₄ alkylene;
   (c) R_{L}^{a} is a bond and R_{L}^{b} is a bond;
   (d) R_{L}^{a} is (C₁-C₄ alkylene)-R_{L}^{T} and R_{L}^{b} is C₁-C₄ alkylene;
   (e) R_{L}^{a} is C₁-C₄alkylene and R_{L}^{b} is R_{L}^{r}-(C₁-C₄ alkylene); or
   (f) R_{L}^{a} is a bond and R_{L}^{b} is (C₁-C₄ alkylene)-R_{L}^{T}-(C₁-C₄ alkylene);

In some embodiments of any of Formulae (I)-(XIII), L¹ is a bivalent linker of Formula (L) or Formula (L-1), wherein:
A_{L} is R_{L}^{a}C(O)R_{L}^{b}, and:
   (a) R_{L}^{a} is C₁-C₄ alkylene and R_{L}^{b} is C₁-C₄ alkylene;
   (b) R_{L}^{a} is C₁-C₄ alkylene and R_{L}^{b} is a bond;
   (c) R_{L}^{a} is a bond and R_{L}^{b} is C₁-C₄ alkylene;
   (d) R_{L}^{a} is a bond and R_{L}^{b} is a bond;
   (e) R_{L}^{a} is (C₁-C₄ alkylene)-R_{L}^{T} and R_{L}^{b} is C₁-C₄ alkylene;
   (f) R_{L}^{a} is C₁-C₄alkylene and R_{L}^{b} is R_{L}^{r}-(C₁-C₄ alkylene); or
   (g) R_{L}^{a} is a bond and R_{L}^{b} is (C₁-C₄ alkylene)-R_{L}^{T}—(C₁-C₄ alkylene);

In some preferred embodiments, A_{L} is R_{L}^{a}C(O)N(R_{L}¹)R_{L}^{b}, and:
(a) R_{L}^{a} is C₁-C₂ alkylene and R_{L}^{b} is C₁-C₂ alkylene;
(b) R_{L}^{a} is C₁-C₂ alkylene and R_{L}^{b} is a bond;
(c) R_{L}^{a} is a bond and R_{L}^{b} is C₁-C₂ alkylene;
(d) R_{L}^{a} is a bond and R_{L}^{b} is a bond;
(e) R_{L}^{a} is (C₁-C₂ alkylene)-R_{L}^{T} and R_{L}^{b} is C₁-C₂ alkylene;
(t) R_{L}^{a} is C₁-C₂alkylene and R_{L}^{b} is R_{L}^{r}-(C₁-C₂ alkylene); or
(g) R_{L}^{a} is a bond and R_{L}^{b} is (C₁-C₂ alkylene)-R_{L}^{T}-(C₁-C₂ alkylene).

In some preferred embodiments, A_{L} is R_{L}^{a}C(O)R_{L}^{b}, and:
(a) R_{L}^{a} is C₁-C₂ alkylene and R_{L}^{b} is C₁-C₂ alkylene;
(b) R_{L}^{a} is C₁-C₂ alkylene and R_{L}^{b} is a bond;
(c) R_{L}^{a} is a bond and R_{L}e is C₁-C₂ alkylene;
(d) R_{L}^{a} is a bond and R_{L}^{b} is a bond;
(e) R_{L}^{a} is (C₁-C₂ alkylene)-R_{L}^{T} and R_{L}^{b} is C₁-C₂ alkylene;
(f) R_{L}^{a} is C₁-C₂alkylene and R_{L}^{b} is R_{L}^{r}-(C₁-C₂ alkylene); or
(g) R_{L}^{a} is a bond and R_{L}^{b} is (C₁-C₂ alkylene)-R_{L}^{r}-(C₁-C₂ alkylene).

In some preferred embodiments of any of the foregoing, R_{L}¹ is H.

In some preferred embodiments of any of the foregoing embodiments of A_{L} in Formula (L) or (L-1), B_{L} is a bivalent moiety selected from the group consisting of:
1) a bond;
2) R_{L}^{a}, where R_{L}^{a} is R_{L}^{r} or R_{L}^{r}-(C₁-C₈ alkylene);
3) R_{L}^{a}OR_{L}^{b}, where R_{L}^{a} is a bond, R_{L}^{b} is R_{L}^{T}, R_{L}^{r}-(C₁-C₈ alkylene) or (C₁-C₈ alkylene)-R_{L}^{T}; and
4) R_{L}^{a}N(R_{L}¹)R_{L}^{b}, where R^{a} is a bond and R_{L}^{b} is R_{L}^{r};

wherein each R_{L}^{r} moiety is C₃-C₁₂ cycloalkylene or 3-12 membered heterocyclene optionally substituted by one or more R_{L}¹, or 5-13 membered heteroarylene optionally substituted by one or more R_{L}^{e}; and

each C₁-C₈ alkylene moiety is optionally substituted by one or more R_{L}^{c}.

In some embodiments where L¹ is a linker of Formula (L) or (L-1), one or more of A_{L}, W_{L}¹, W_{L}² and B_{L}, at each occurrence, is R_{L}^{a}, where R_{L}^{a} is optionally substituted R_{L}^{r}, (C₁-C₈ alkylene)-R_{L}¹, R_{L}^{r}-(C₁—C₈ alkylene) or (C₁-C₈ alkylene)-R_{L}^{r}-(C₁-C₈ alkylene), and each R_{L}^{r} is independently selected from the group consisting of optionally substituted C₃-C₁₂ cycloalkylene, 3-12 membered heterocyclene, C₆-C₂ arylene, and 5-13 membered heteroarylene.

In some embodiments where L¹ is a linker of Formula (L) or (L-1), one or more of A_{L}, W_{L}¹, W_{L}², and B_{L}, at each occurrence, is R_{L}^{a}-R_{L}^{b}, where each R_{L}^{a} and R_{L}^{b} is independently selected from the group consisting of a bond, R_{L}^{r}, (C₁-C₈ alkylene)-R_{L}^{r}, R_{L}^{r}-(C₁-C₈ alkylene), (C₁-C₈ alkylene)-R_{L}^{r}-(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, each optionally substituted as described herein.

In some embodiments where L¹ is a linker of Formula (L) or (L-1), one or more of A_{L}, W_{L}¹, W_{L}², and B_{L}, at each occurrence, is R_{L}^{a}-R_{L}^{b}, wherein one of R_{L}^{a} and R_{L}^{b} is optionally substituted C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene and the other of R_{L}^{a} and R_{L}^{b} is R_{L}^{r} wherein R_{L}^{r} is independently selected from the group consisting of optionally substituted C₃-C₁₂ cycloalkylene, 3-12 membered heterocyclene, C₆-C₁₂ arylene, and 5-13 membered heteroarylene.

In some embodiments where L¹ is a linker of Formula (L) or (L-1), one or more of A_{L}, W_{L}¹, W_{L}², and B_{L}, at each occurrence, is R_{L}^{a}, where R_{L}^{a} is optionally substituted C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene.

In some embodiments where L¹ is a linker of Formula (L) or (L-1), one or more of A_{L}, W_{L}¹, W_{L}², and B_{L}, at each occurrence, is R_{L}^{a}, where R_{L}^{a} is R_{L}^{r} and R_{L}^{r} is optionally substituted C₃-C₁₂ cycloalkylene, 3-12 membered heterocyclene, C₆-C₁₂ arylene, and 5-13 membered heteroarylene. In some such embodiments, R_{L}^{r} is optionally substituted C₃-C₁₂ cycloalkylene. In some such embodiments, R_{L}^{r} is optionally substituted 3-12 membered heterocyclene. In some such embodiments, R_{L}^{r} is optionally substituted C₆-C₁₂ arylene or 5-13 membered heteroarylene.

In some embodiments where L¹ is a linker of Formula (L) or (L-1), A_{L}, W_{L}¹, W_{L}², and B_{L}, at each occurrence, is independently optionally substituted C₁-C₈ alkylene. In some embodiments, A_{L}, W_{L}¹, W_{L}², and B_{L}, at each occurrence, is optionally substituted C₂-C₈ alkenylene. In some embodiments, A_{L}, W_{L}¹, W_{L}², and B_{L}, at each occurrence, is optionally substituted C₂-C₈ alkynylene. In some embodiments, A_{L}, W_{L}¹, W_{L}², and B_{L}, at each occurrence, is optionally substituted C₂-C₈ heteroalkylene. In some embodiments, A_{L}, W_{L}¹, W_{L}², and B_{L}, at each occurrence, is optionally substituted C₃-C₈ heteroalkenylene. In some embodiments, A_{L}, W_{L}¹, W_{L}², and B_{L}, at each occurrence, is optionally substituted C₃-C₈ heteroalkynylene.

In some embodiments where L¹ is a linker of Formula (L), m_{L} is 0 and W_{L}¹ and W_{L}² are null (i.e., the linker has the structure of Formula (L-1)).

In some embodiments where L¹ is a linker of Formula (L) or (L-1), each R_{L}^{a} and R_{L}^{b} is independently selected from the group consisting of a bond, R_{L}^{r}, (C₁-C₈ alkylene)-R_{L}^{r}, R_{L}^{T}-(C₁-C₈alkylene), (C₁-C₈ alkylene)-R_{L}^{r}-(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, where each said C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene is optionally substituted by one or more R_{L}^{T}. In such embodiments, each Ru is independently selected from the group consisting of C₃-C₁₂ cycloalkylene, 3-12 membered heterocyclene, C₆-C₁₂ arylene, and 5-13 membered heteroarylene, where each said C₃-C₁₂ cycloalkylene or 3-12 membered heterocyclene is optionally substituted by one or more R_{L}^{d}, and each said C₆-C₁₂ arylene or 5-13 membered heteroarylene is optionally substituted by one or more R_{L}^{e}. In some such embodiments, one or both of R_{L} and R_{L}^{b} is R_{L}^{r}, where R_{L}^{r} is optionally substituted C₃-C₁₀ cycloalkylene, 3-10 membered heterocyclene, C₆-C₁₂ arylene, or 5-13 membered heteroarylene.

In some embodiments, A_{L} is a bivalent moiety selected from the group consisting of R_{L}, R_{L}^{a}-R_{L}^{b}R_{L}^{a}C(O)R_{L}^{b}, R_{L}C(O)N(R_{L}¹)R_{L}^{b}, R_{L}^{a}N(R_{L}¹)C(O)R_{L}^{b}, R_{L}^{a}OR_{L}^{b}, and R_{L}^{a}N(R_{L}¹)R_{L}^{b}.

In some such embodiments: A_{L} is R_{L}^{a}C(O)NHR_{L}^{b}, R_{L}^{a} is C₁-C₄ alkylene, and R_{L}^{b} is C₁-C₄ alkylene; A_{L} is R_{L}^{a}C(O)NHR_{L}^{b}, R_{L}^{a} is (C₁-C₄ alkylene)-R_{L}^{r}, and R_{L}^{b} is C₁-C₄ alkylene; A_{L} is R_{L}^{a}C(O)NHR_{L}^{b}, R_{L}^{a} is C₁-C₄ alkylene, and R_{L}^{b} is R_{L}^{r}-(C₁-C₄ alkylene); A_{L} is R_{L}^{a}C(O)NHR_{L}^{b}, R_{L}^{a} is a bond, and R_{L}^{b} is (C₁-C₄ alkylene)-R_{L}^{r}-(C₁-C₄ alkylene); A_{L} is R_{L}^{a}C(O)NHR_{L}^{b}, R_{L}^{a} is C₁-C₄ alkylene, and R_{L}^{b} is a bond; A_{L} is R_{L}^{a}C(O)NHR_{L}^{b}, R_{L} is a bond, and R_{L}^{b} is C₁-C₄ alkylene; A_{L} is R_{L}^{a}C(O)NHR_{L}^{b}, R_{L} is a bond, and R_{L}^{b} is a bond; A_{L} is R_{L}^{a}N(R_{L}¹)C(O)R_{L}^{b}, R_{L}^{a} is C₁-C₄ alkylene, and R_{L}^{b} is C₁-C₄ alkylene; A_{L} is R_{L}^{a}N(R_{L}¹)C(O)R_{L}^{b}, R_{L}^{a} is C₁-C₄ alkylene, and R_{L}^{b} is a bond; A_{L} is R_{L}^{a}N(R_{L}¹)C(O)R_{L}^{b}, R_{L}^{a} is a bond, and R_{L}^{b} is C₁-C₄ alkylene; A_{L} is R_{L}^{a}N(R_{L})C(O)R_{L}^{b}, R_{L}^{a} is a bond, and R_{L}^{b} is a bond; A_{L} is R_{L}^{a}NHC(O)R_{L}, R_{L}^{a} is C₁-C₄ alkylene, and R_{L}^{b} is C₁-C₄ alkylene; A_{L} is R_{L}^{a}C(O)R_{L}^{b}, R_{L} is(C₁-C₄ alkylene)-R^{r}, and R_{L}^{b} is a bond; A_{L} is R_{L}^{a}C(O)R_{L}^{b}, R_{L}^{a} is C₁-C₄ alkylene, and R_{L}^{b} is R_{L}^{r}-(C₁-C₄ alkylene); A_{L} is R_{L}^{a}C(O)R_{L}^{b}, R_{L} is C₁-C₄ alkylene, and R_{L}^{b} is C₁-C₄ alkylene; A_{L} is R_{L}^{a}C(O)R_{L}^{b}, R_{L}^{a} is C₁-C₄ alkylene, and R_{L}^{b} is a bond; A_{L} is R_{L}^{a}C(O)R_{L}^{b}, R_{L}^{a} is a bond, and R_{L}^{b} is C₁-C₄ alkylene; A_{L} is R_{L}^{a}C(O)R_{L}^{b}, R_{L}^{a} is a bond, and R_{L}^{b} is a bond; A_{L} is R_{L}^{a}-R_{L}^{b}, R_{L}^{a} is C₁-C₄ alkylene, and R_{L}^{b} is C₁-C₄ alkylene; A_{L} is R_{L}^{a}-R_{L}^{b}, R_{L}^{a} is (C₁-C₄ alkylene)-R_{L}^{r}, and R_{L}^{b} is C₁-C₄ alkylene; A_{L} is R_{L}^{a}NHR_{L}^{b}, R_{L}^{a} is C₁-C₈ alkylene, and R_{L}^{b} is a bond; A_{L} is R_{L}^{a}NHR_{L}^{b}, R_{L}^{a} is C₁-C₄ alkylene, and R_{L}^{b} is C₁-C₄ alkylene; or A_{L} is R_{L}^{a}NHR_{L}^{b}, R_{L}^{a} is a bond, and R_{L}^{b} is C₁-C₈ alkylene. In some embodiments of A_{L}, each R_{L}^{r} moiety is C₃-C₁₂ cycloalkylene or 3-12 membered heterocyclene, each optionally substituted by one or more R_{L}^{d}. In other such embodiments of A_{L}, each R_{L}^{r} moiety is C₆-C₁₂ arylene or 5-13 membered heteroarylene, each optionally substituted by one or more R_{L}^{e}.

In some embodiments, A_{L} is a bond, —C(═O)—, —C(═O)NH—, —NH—, —NH—C(═O)—, —O—, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C—C₈ alkylene)-O—, —C₁-C₈ alkylene-, —C₂-C₈ alkenylene, or —C₂-C₈ alkynylene-. In some embodiments, A_{L} is a bond, —(C₁-C₈ alkylene)-C(═O)NH—, —(C₁-C₈ alkylene)-C(═O)—, —(C₁-C₈ alkylene)NH—, —(C₁-C₈ alkylene)-NH—C(═O)—, —(C₁-C₈ alkylene)-O—, or —C₁-C₈ alkylene-. In some embodiments, A_{L} is a bond. In some embodiments, A_{L} is —C(═O)—. In some embodiments, A_{L} is —C(═O)NH—. In some embodiments, A_{L} is —NH—. In some embodiments, A_{L} is —NH—C(═O)—. In some embodiments, A_{L} is —O—. In some embodiments, A_{L} is —(C₁-C₈ alkylene)-C(═O)NH—. In some embodiments, A_{L} is —(C₁-C₈ alkylene)-C(═O)—. In some embodiments, A_{L} is —(C₁-C₈ alkylene)NH—. In some embodiments, A_{L} is —(C₁-C₈ alkylene)-NH—C(═O)—. In some embodiments, A_{L} is —(C₁-C₈ alkylene)-O—. In some embodiments, A_{L} is —C₁-C₈ alkylene-. In some embodiments, A_{L} is —C₂-C₈ alkenylene. In some embodiments, A_{L} is —C₂-C₈ alkynylene-.

In some embodiments, B_{L} is a bivalent moiety selected from the group consisting of a bond, R_{L}^{a}, R_{L}^{a}-R_{L}^{b}, R_{L}^{a}C(O)R_{L}^{b}, R_{L}^{a}C(O)N(R_{L}¹)R_{L}^{b}, R_{L}^{a}N(R_{L}¹)C(O)R_{L}^{b}, R_{L}^{a}OR_{L}^{b} and R_{L}^{a}N(R_{L}¹)R_{L}^{b}. In some embodiments, B_{L} is a bivalent moiety selected from the group consisting of a bond, R_{L}^{a}, R_{L}^{a}OR_{L}^{b} and R_{L}^{a}N(R_{L}¹)R_{L}^{b}. In some embodiments, B_{L} is a bivalent moiety selected from the group consisting of a bond, R_{L}^{a}, R_{L}^{a}OR_{L}^{b} and R_{L}^{a}N(R_{L}¹)R_{L}^{b}, wherein R_{L}^{a} is R_{L}^{r} or R_{L}^{r}-(C₁-C₄ alkylene).

In some embodiments, B_{L} is R_{L}^{a}OR_{L}^{b}, R_{L}^{a} is a bond, and R_{L}^{b} is R_{L}^{r}, (C₁-C₈ alkylene)-R_{L}^{r}, or R_{L}^{r}—(C₁-C₈ alkylene). In some embodiments, B_{L} is R_{L}^{a}N(R_{L}¹)R_{L}^{b}, R_{L}^{a} is a bond, and R_{L}^{b} is R_{L}^{r}, (C₁-C₈ alkylene)-R_{L}^{r}, or R_{L}^{r}-(C₁-C₈ alkylene). In some embodiments, B_{L} is a bond. In other embodiments, B_{L} is R_{L}^{a}, where R_{L}^{a} is R_{L}^{r}, (C₁-C₈ alkylene)-R_{L}^{r}, R_{L}^{r}-(C₁-C₈ alkylene) or (C₁-C₈ alkylene)-R_{L}^{r}-(C₁-C₈ alkylene). In some such embodiments, B_{L} is R_{L}^{a}, where R_{L}^{a} is R_{L}^{r}, (C₁-C₄ alkylene)-R_{L}^{r}, R_{L}^{r}-(C₁-C₄ alkylene) or (C₁-C₄ alkylene)-R_{L}^{r}-(C₁-C₄ alkylene).

In some embodiments of B_{L}, each R_{L}^{r} moiety is C₃-C₁₂ cycloalkylene or 3-12 membered heterocyclene, each optionally substituted by one or more R_{L}^{d}. In some embodiments of B_{L}, each R_{L}^{T} moiety is C₆-C₁₂ arylene or 5-13 membered heteroarylene, each optionally substituted by one or more R_{L}^{e}.

In some embodiments, B_{L} is a bond, —C(═O)—, —C(═O)NH—, —NH—, —NH—C(═O)—, —O—, —C₁-C₈ alkylene-, —C₂-C₈ alkenylene-, —C₂-C₈ alkynylene-, —NH—(C₁-C₈ alkylene)-, —O—(C₁-C₈ alkylene)-, —C(═O)-(C₁-C₈ alkylene)-, —C(═O)NH—(C₁-C₈ alkylene)-, or —NH—C(═O)-(C₁-C₈ alkylene)-. In some embodiments, B_{L} is a bond, —(C₁-C₈ alkylene)-, —NH—(C₁-C₈ alkylene)-, —O—(C₁-C₈ alkylene)-, —C(═O)-(C₁-C₈ alkylene)-, —C(═O)NH—(C₁-C₈ alkylene)-, or —NH—C(═O)-(C₁-C₈ alkylene)-. In some embodiments, B_{L} is a bond. In some embodiments, B_{L} is —C(═O)—. In some embodiments, B_{L} is —C(═O)NH—. In some embodiments, B_{L} is —NH—. In some embodiments, B_{L} is —NH—C(═O)—. In some embodiments, B_{L} is —O—. In some embodiments, B_{L} is —C₁-C₈ alkylene-. In some embodiments, B_{L} is —C₂-C₈ alkenylene-. In some embodiments, B_{L} is —C₂-C₈ alkynylene-. In some embodiments, B_{L} is —NH—(C₁-C₈ alkylene)-. In some embodiments, B_{L} is —O—(C₁-C₈ alkylene)-. In some embodiments, B_{L} is —C(═O)-(C₁-C₈ alkylene)-. In some embodiments, B_{L} is —C(═O)NH—(C₁-C₈ alkylene)-. In some embodiments, B_{L} is —NH—C(═O)-(C₁-C₈ alkylene)-.

In some embodiments, where present, each W_{L}¹ is independently R_{L}^{r} or C₁-C₃ alkylene; and each W_{L}² is independently a bond, O, or NH. In some such embodiments, each W_{L}¹ is independently R_{L}^{r}; and each W_{L}² is independently a bond, O, or NH. In some such embodiments, each W_{L}¹ is independently C₁—, C₂—, or C₃-alkylene; and each W_{L}² is independently a bond, O, or NH. In some such embodiments, each W_{L}¹ is independently C₁-, C₂- or C₃-alkylene; and each W_{L}² is independently O or NH. In other such embodiments, each W_{L}¹ is independently C₁-, C₂- or C₃-alkylene; and each W_{L}² is independently O. In still other embodiments, each W_{L}¹ is independently C₁-, C₂- or C₃-alkylene; and each W_{L}² is independently NH.

In some embodiments, where present, each W_{L}¹ is independently a bond, O, or NH; and each W_{L}² is independently R_{L}^{r} or C₁-C₃ alkylene. In some such embodiments, each W_{L}¹ is independently a bond, O, or NH; and each W_{L}² is independently R_{L}^{r}. In some such embodiments, each W_{L}¹ is independently a bond, O, or NH; and each W_{L}² is independently C₁-, C₂- or C₃-alkylene. In other such embodiments, each W_{L}¹ is independently a bond or O; and each W_{L}² is independently C₁-, C₂- or C₃-alkylene. In some such embodiments, each W_{L}¹ is independently O; and each W_{L}² is independently C₁-, C₂- or C₃-alkylene. In some such embodiments, each W_{L}¹ is independently NH; and each W_{L}² is independently C₁-, C₂- or C₃-alkylene.

In some embodiments, where present, each —W_{L}¹-W_{L}²— is independently —CH₂CH₂O— or —CH₂—. In some embodiments, each —W_{L}¹-W_{L}²— is independently —CH₂CH₂O—. In some embodiments, each —W_{L}¹-W_{L}²— is independently —CH₂—.

In some embodiments, at least one R_{L}^{r} moiety in A_{L} or B_{L} is C₃-C₁₂ cycloalkylene or 3-12 membered heterocyclene, each optionally substituted by one or more R_{L}^{d}. In other embodiments, at least one R_{L}^{r} moiety in A_{L} or B_{L} is C₆-C₁₂ arylene or 5-13 membered heteroarylene, each optionally substituted by one or more R_{L}^{c}.

In some embodiments, each R_{L}^{r}moiety is independently selected from C₃-C₁₀ cycloalkylene or 3-12 membered heterocyclene, each optionally substituted by one or more R_{L}^{d}. In some embodiments, each R_{L}^{r} moiety is independently selected from C₆-C₁₂ arylene or 5-13 membered heteroarylene, each optionally substituted by one or more R_{L}^{e}.

In some embodiments, each R_{L}^{r} moiety is optionally substituted C₃-C₁₂ cycloalkylene. In some embodiments, each R_{L}^{r} moiety is optionally substituted 3-12 membered heterocyclene. some embodiments, each R_{L}^{r} moiety is optionally substituted C₆-C₁₂ arylene. In some embodiments, each R_{L}^{r} moiety is optionally substituted 5-13 membered heteroarylene.

In some embodiments, m_{L} is an integer selected from 0-15, 1-14, 1-13, 1-12, 1-11, 1-10, 1-9, 1-8, 1-7, 1-6, 1-5, 1-4, 1-3, or 1-2. In some embodiments, m_{L} is an integer selected from 0-13. In some embodiments, m_{L} is an integer selected from 0-12. In some embodiments, m_{L} is an integer selected from 0-11. In some embodiments, m_{L} is an integer selected from 0-10. In some embodiments, m_{L} is an integer selected from 0-9. In some embodiments, m_{L} is an integer selected from 0-8. In some embodiments, m_{L} is an integer selected from 0-7. In some embodiments, m_{L} is an integer selected from 0-6. In some embodiments, m_{L} is an integer selected from 0-5. In some embodiments, m_{L} is an integer selected from 0-4. In some embodiments, m_{L} is an integer selected from 0-3. In some embodiments, m_{L} is an integer selected from 0, 1, 2, 3, 4, 5, 6, 7, 8, 9, 10, 11, 12, 13, 14, or 15.

In some embodiments of any of Formulae (I)-(XIII), when L¹ is a linker of Formula (L), the linker comprises one or more optionally substituted polyethylene glycol (PEG) units. In some embodiments, the PEG is optionally substituted and 1-5, 2-7, 2-10, 2-20, 5-25, or 4-30 —(O—CH₂CH₂)— units in length. In some embodiments, L¹ comprises an optionally substituted alkylene. In some embodiments, L¹ is a straight chain alkylene. In some embodiments, L¹ comprises optionally substituted C₂-C₃₀, C₂-C₂₅, C₃-C₂₅, C₄-C₁₀, C₆-C₁₂, C₆-C₁₈, or C₄-C₂₀ alkyl units. In some embodiments, L¹ comprises an optionally substituted carbocycle ring. In some embodiments, L¹ comprises an optionally substituted heterocycle ring. In some embodiments, L¹ comprises an optionally substituted C₆-C₁₂ aryl ring. In some embodiments, L¹ comprises an optionally substituted 5-13 membered heteroaryl ring. In some embodiments, L¹ comprises one or more ethers. In some embodiments, L¹ comprises one or more C₂-C₃₀, C₂-C₂₅, C₃-C₂₅, C₄-C₁₀, C₆-C₁₂, C₆-C₁₈, or C₄-C₂₀ alkyl ether units. In some embodiments, L¹ comprises one or more amines. In some embodiments, L comprises one or more C₂-C₃₀, C₂-C₂₅, C₃-C₂₅, C₄-C₁₀, C₆-C₁₂, C₆-C₁₈, or C₄-C₂₀ alkylamino units. In some embodiments, L¹ comprises optionally substituted 1-5, 2-7, 2-10, 2-20, 5-25, or 4-30 —(NH—CH₂CH₂)-units.

In some embodiments, L¹ comprises one or more amides. In some embodiments, L¹ comprises one or more sulfonamides. In some embodiments, L¹ comprises one or more carbamates. In some embodiments, L comprises one or more carbonates.

In some embodiments of any of Formulae (I)-(XIII), L¹ is a linker of Formula (L) or (L-1) comprising at least one ring moiety, R_{L}^{r}, wherein R_{L}^{r} may be monocyclic, fused or spirocyclic C₃-C₁₂ cycloalkylene or 3-12 membered heterocyclene, or monocyclic or fused C₆-C₁₂ arylene or 5-13 membered heteroarylene, each optionally substituted as described. In other embodiments of any of Formulae (I)-(XIII), L¹ is a linker of Formula (J) comprising at least one ring moiety, —Ar″— or -Cy″-, as described herein. Examples of ring moieties suitable for inclusion in linkers of Formula (L), (L-1) or (J) include the moieties of Formula (L-2), Formula (L-3), Formula (L-4), Formula (L-5) and Formula (L-6), and embodiments thereof described herein.

In some embodiments of any of Formulae (I)-(XIII), L¹ comprises one or more rings selected from the group consisting of Formula (L-2), Formula (L-3), Formula (L-4), Formula (L-5) and Formula (L-6):

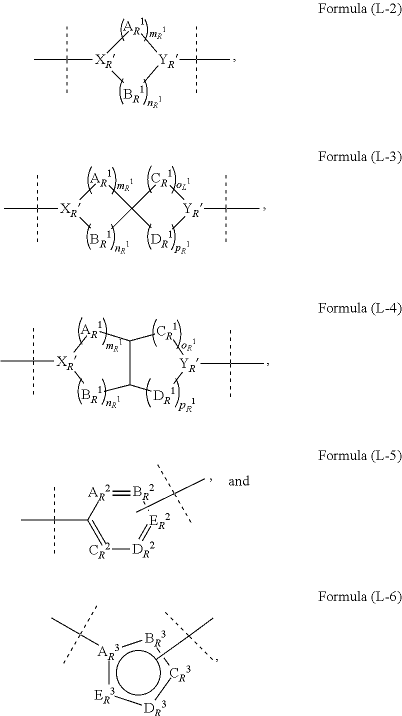

wherein:
X_{R}′ and Y_{R}′ are independently selected from N and CR_{R}^{b};
A_{R}¹, B_{R}¹, C_{R}¹ and D_{R}¹, at each occurrence, are independently selected from a bond, O, CO, SO, SO₂, C(O)NR_{R}^{b}, S(O)₂NR_{R}^{b}, NR_{R}^{b} and CR_{R}^{b}R_{R}^{c};
A_{R}², B_{R}², C_{R}², D_{R}², and E_{R}², at each occurrence, are independently selected from N and CR_{R}^{b}; A_{R}³, at each occurrence, is independently selected from N and C, and B_{R}³, C_{R}³, D_{R}³, and E_{R}³, at each occurrence, are independently selected from N, O, S, NR_{R}^{b} and CR_{R}^{b};
R_{R}^{b} and R_{R}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-12 membered carbocyclyl, optionally substituted 3-12 membered cycloalkoxy, optionally substituted 3-12 membered carbocyclylamino, optionally substituted 4-12 membered heterocyclyl, optionally substituted C₆-C₁₂ aryl, and optionally substituted 5-13 membered heteroaryl; or
two R_{R}^{b} and R_{R}^{c} together with the atom(s) to which they are attached optionally form a C₃-C₁₂ carbocyclyl or 3-12 membered heterocyclyl; and
m_{R}¹, n_{R}¹, o_{R}¹ and p_{R}¹ are independently selected from 0, 1, 2, 3, 4 and 5.

In some embodiments of any of Formulae (I)-(XIII), the linker L¹ comprises one or more rings selected from the group consisting of:

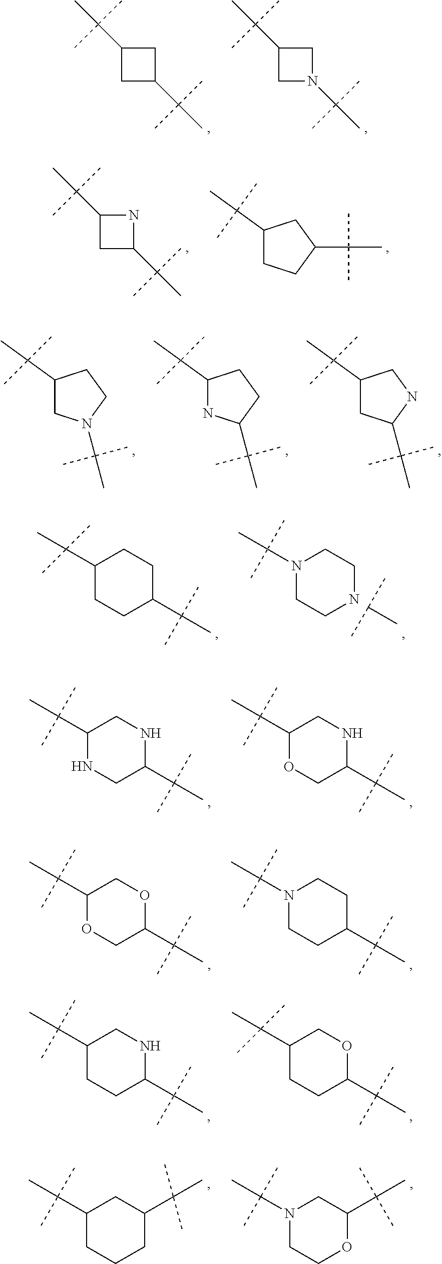

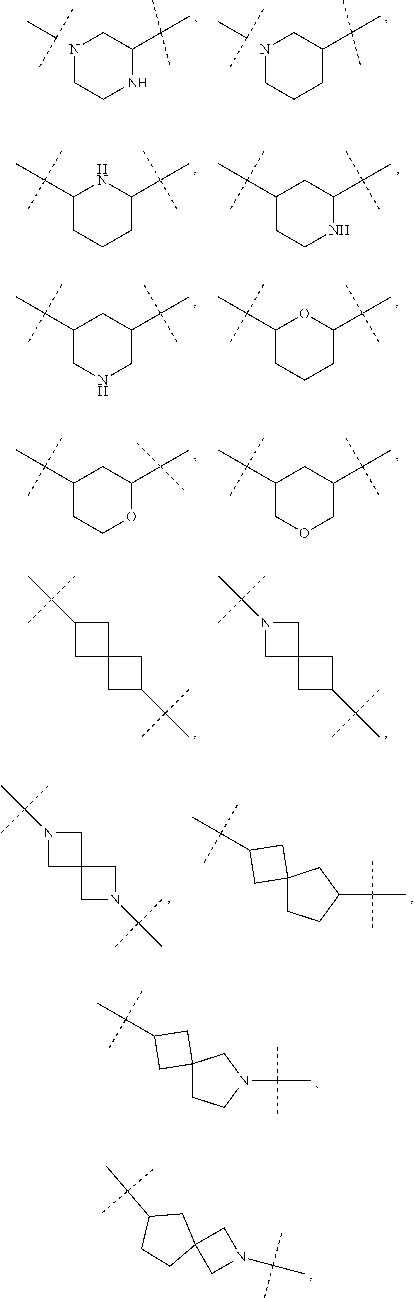

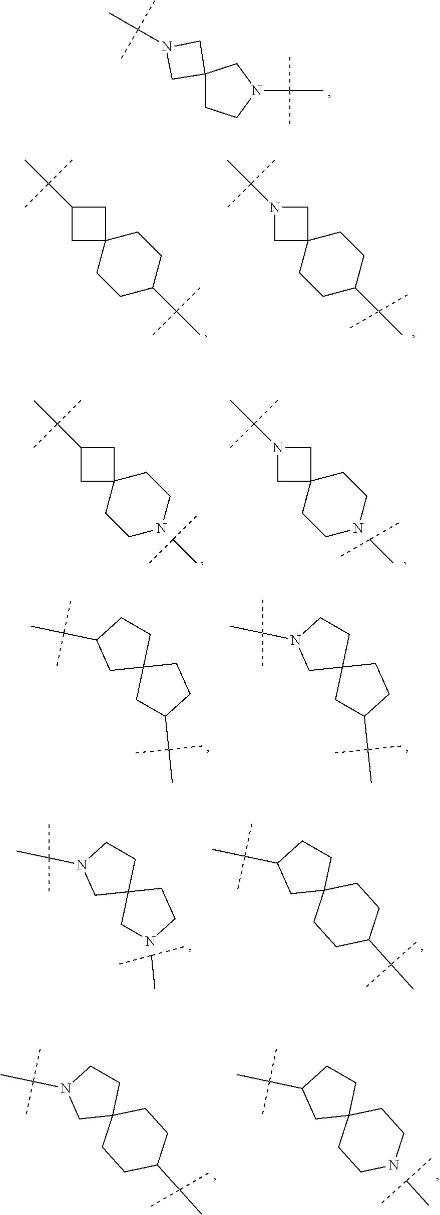

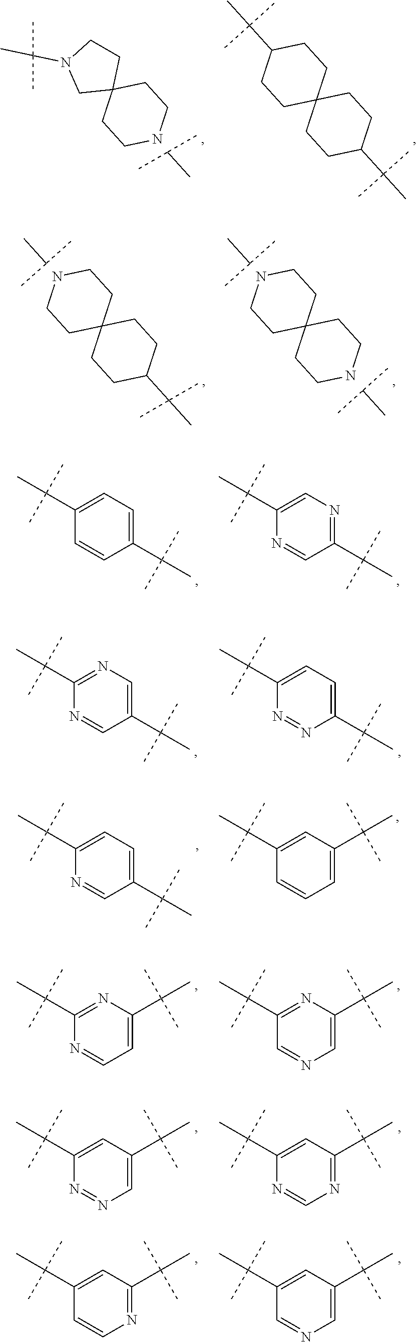

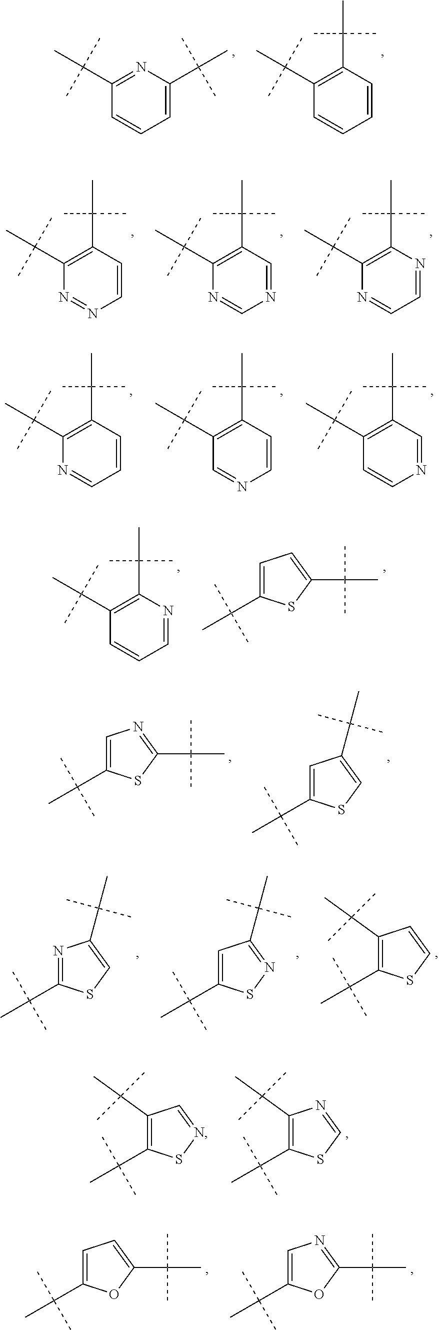

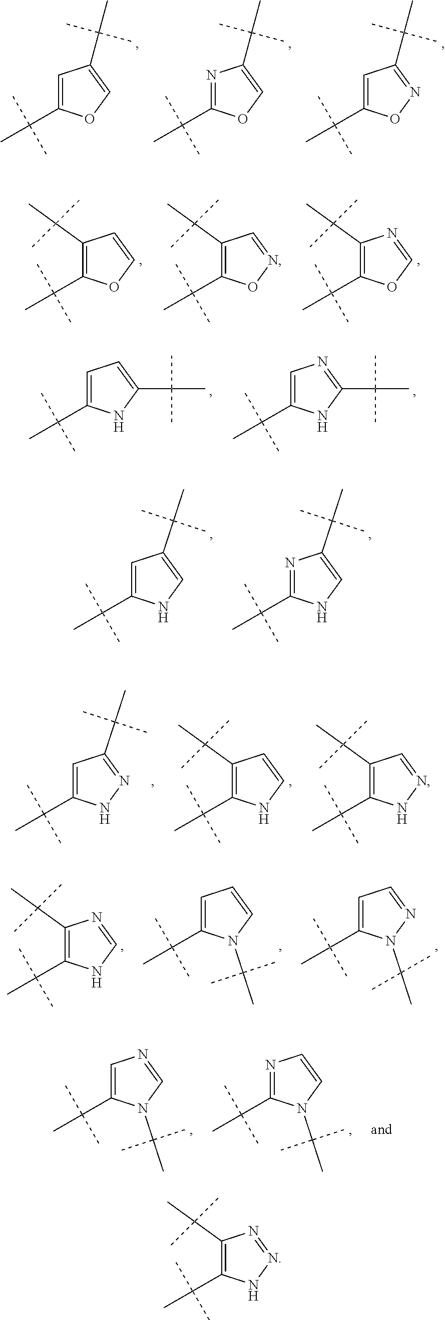

In some such embodiments, the linker L¹ comprises one or more rings selected from the group consisting of:

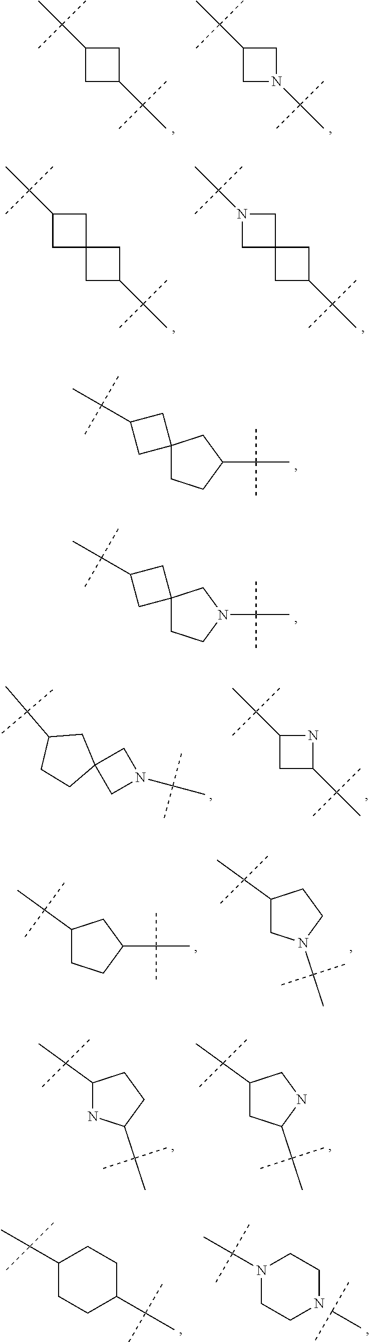

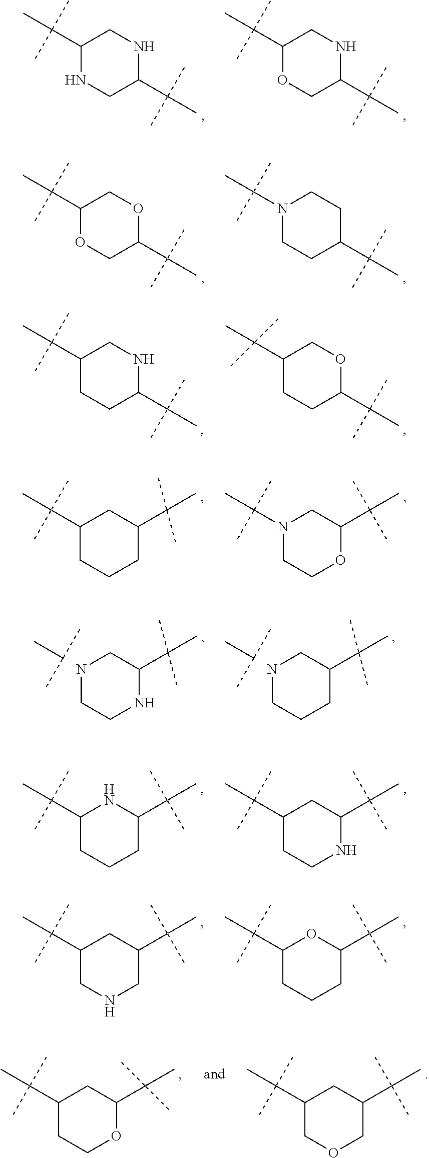

In some such embodiments, the linker L¹ comprises one or more rings selected from the group consisting of:

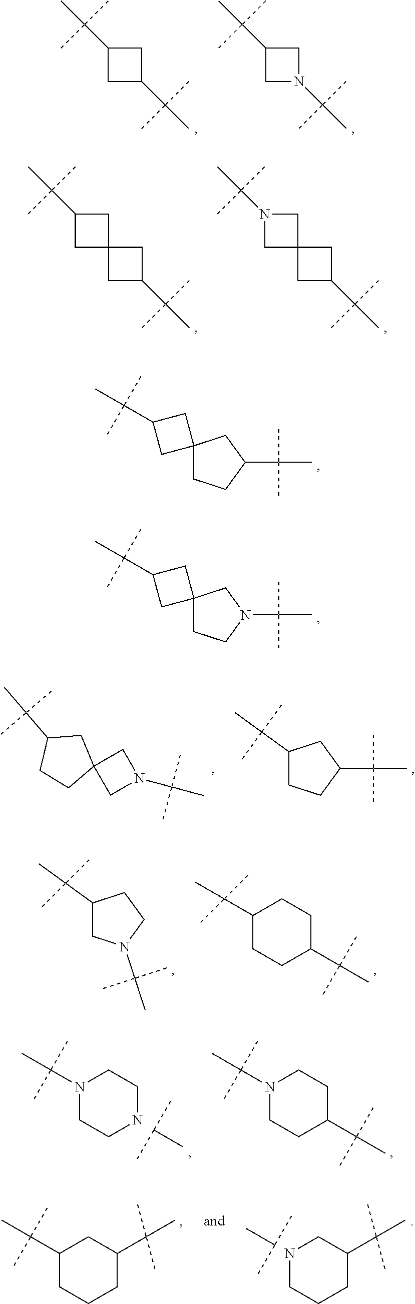

In some such embodiments, the linker L¹ comprises one or more rings selected from the group consisting of:

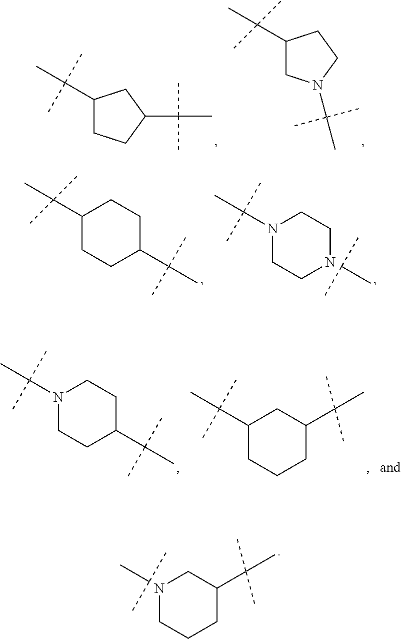

In some such embodiments, the linker L¹ comprises one or more rings selected from the group consisting of:

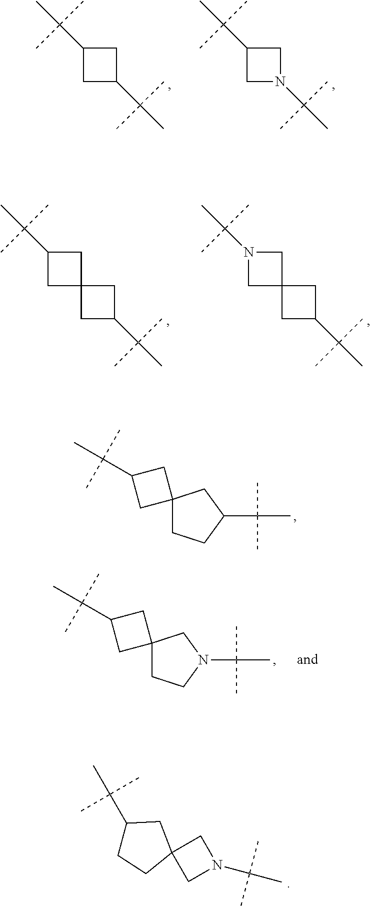

When the linker L¹ comprises one or more rings as described above, such rings may be incorporated as R_{L}^{r} moieties in compounds where L¹ a bivalent linker of Formula (L) or (L-1).

Alternatively, such rings may be incorporated as —Ar″— or -Cy″- moieties in compounds where L¹ a bivalent linker of Formula (J).

In some embodiments, L¹ is a linker of Formula (L) or (L-1) comprising one or more R_{L} moieties selected from the group consisting of Formula (L-2), Formula (L-3), Formula (L-4), Formula (L-5) and Formula (L-6). In other embodiments, L¹ is a linker of Formula (J) comprising one or more —Ar″— or -Cy″- moieties selected from the group consisting of Formula (L-2), Formula (L-3), Formula (L-4), Formula (L-5) and Formula (L-6).

In some embodiments, L¹ is selected from —(CH₂)ₚ₁C(═O)NH—(CH₂CH₂O)ₚ₂—(CH₂)ₚ₃—, —(CH₂)ₚ₁C(═O)NH(CH₂)ₚ₂—, —(CH₂)ₚ₁NHC(═O)-(CH₂CH₂O)ₚ₂—(CH₂)₃—, —(CH₂)ₚ₁NHC(═O)-(CH₂)ₚ₂—, —(CH₂)ₚ₁C(═O)-(CH₂CH₂O)ₚ₂—(CH₂)ₚ₃—, —(CH₂)ₚ₁C(═O)-(CH₂)ₚ₂—, —(CH₂)ₚ₁NH(CH₂CH₂O)ₚ₂—(CH₂)ₚ₃—, —(CH₂)ₚ₁NH(CH₂)ₚ₂—, —(CH₂CH₂O)ₚ₂—(CH₂)ₚ₃—, or —(CH₂)ₚ₂—; wherein p1 is an integer selected from 0 to 9; p2 is an integer selected from 0 to 15; and p3 is an integer selected from 0 to 9.

In some embodiments, the linker L¹ is —(CH₂)₀₋₃—(C₃-C₁₂ cycloalkyl)-(CH₂)₀₋₄—, —(CH₂)₀₋₃-(3-12 membered heterocyclyl)-(CH₂)₀₋₄—, —(CH₂)₀₋₃—(C₆-C₁₂ aryl)-(CH₂)₀₋₄—, —(CH₂)₀₋₃-(5-13 membered heteroaryl)-(CH₂)₀₋₄—, —(CH₂)₀₋₃—C(═O)-(CH₂)₀₋₃—(C₃-C₁₂ cycloalkyl)-(CH₂)₀₋₄—, —(CH₂)₀₋₃—C(═O)-(CH₂)₀₋₃-(3-12 membered heterocyclyl)-(CH₂)₀₋₄—, —(CH₂)₀₋₃—C(═O)-(CH₂)₀₋₃—(C₆-C₁₂ aryl)-(CH₂)₀₋₄—, —(CH₂)₀₋₃—C(═O)-(CH₂)₀₋₃-(5-13 membered heteroaryl)-(CH₂)₀₋₄—, —(CH₂)₀₋₃—C(═O)NH—(CH₂)₀₋₃—(C₃-C₁₂ cycloalkyl)-(CH₂)₀₋₄—, —(CH₂)₀₋₃—C(═O)NH—(CH₂)₀₋₃-(3-12 membered heterocyclyl)-(CH₂)₀₋₄—, —(CH₂)₀₋₃—C(═O)NH—(CH₂)₀₋₃—(C₆-C₁₂ aryl)-(CH₂)₀₋₄—, —(CH₂)₀₋₃—C(═O)NH—(CH₂)₀₋₃-(5-13 membered heteroaryl)-(CH₂)₀₋₄—, —(CH₂)₀₋₃—NHC(═O)-(CH₂)₀₋₃—(C₃-C₁₂ cycloalkyl)-(CH₂)₀₋₄—, —(CH₂)₀₋₃—NHC(═O)—(CH₂)₀₋₃-(3-12 membered heterocyclyl)-(CH₂)₀₋₄—, —(CH₂)₀₋₃—NHC(═O)-(CH₂)₀₋₃—(C₆-C₁₂ aryl)-(CH₂)₀₋₄—, or —(CH₂)₀₋₃—NHC(═O)-(CH₂)₀₋₃-(5-13 membered heteroaryl)-(CH₂)₀₋₄—.

In some embodiments, the linker L¹ is —(CH₂)₀₋₃—(C₃-C₁₂ cycloalkyl)-(CH₂)₀₋₄—, —(CH₂)₀₋₃-(3-12 membered heterocyclyl)-(CH₂)₀₋₄—, —(CH₂)₀₋₃—C(═O)-(CH₂)₀₋₃—(C₃-C₁₂ cycloalkyl)-(CH₂)₀₋₄—, —(CH₂)₀₋₃—C(═O)-(CH₂)₀₋₃-(3-12 membered heterocyclyl)-(CH₂)₀₋₄—, —(CH₂)₀₋₃—C(═O)NH—(CH₂)₀₋₃—(C₃-C₁₂ cycloalkyl)-(CH₂)₀₋₄—, —(CH₂)₀₋₃—C(═O)NH—(CH₂)₀₋₃-(3-12 membered heterocyclyl)-(CH₂)₀₋₄—, —(CH₂)₀₋₃—NHC(═O)-(CH₂)₀₋₃—(C₃-C₁₂ cycloalkyl)-(CH₂)₀₋₄—, or —(CH₂)₀₋₃—NHC(═O)-(CH₂)₀₋₃-(3-12 membered heterocyclyl)-(CH₂)₀₋₄—.

In some embodiments, the linker L¹ is —C(═O)-(CH₂)₁₋₈—, —(CH₂)₁₋₉—, —(CH₂)₁₋₂—C(═O)—NH—(CH₂)₂₋₉—, —(CH₂)₁₋₂—C(═O)—NH—(CH₂)₁₋₃—(OCH₂CH₂)₁₋₇—, —(CH₂)₀₋₁—C(═O)-(CH₂)₁₋₃—(OCH₂CH₂)₁₋₇—, —C(═O)-(CH₂)₀₋₃-(alkenylene)-(CH₂)₀₋₃—, —C(═O)-(CH₂)₀₋₃-(alkynylene)-(CH₂)₀₋₃—, —C(═O)-(CH₂)₀₋₃-(3-8 membered carbocyclyl)-(CH₂)₀₋₃—, —C(═O)-(CH₂)₀₋₃-(3-8 membered heterocarbocyclyl)-(CH₂)₀₋₃—, (CH₂)₀₋₃-(alkenylene)-(CH₂)₀₋₃—, —(CH₂)₀₋₃-(alkynylene)-(CH₂)₀₋₃—, —(CH₂)₀₋₃-(3-8 membered carbocyclyl)-(CH₂)₀₋₃—, or —(CH₂)₀₋₃-(3-8 membered heterocarbocyclyl)-(CH₂)₀₋₃—.

In some embodiments of any of Formulae (I)-(XIII), L¹ is a bivalent linker of Formula (J):

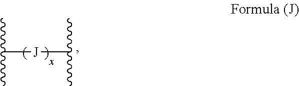

wherein:
x is an integer from 1 to 30;
each unit -(J)- is independently selected from the group consisting of: a bond, —N(R¹³)—, —(C(R¹⁴)₂)₁₋₃-, —O—, —C(O)—, —C(═N(R¹³))—, —C(S)—, —C(R¹⁴)═C(R¹⁴)—, —C≡C—, —S—, —S(O)—, —S(O)₂—, —Ar″— and -Cy″- provided two —O— and/or —S— are not contiguous;
each —Ar″— is independently C₆-C₁₀ arylene or 5-10 membered heteroarylene, each optionally substituted with one or more R¹⁵;
each -Cy″- is independently a monocyclic, fused or spirocyclic C₃-C₁₂ cycloalkylene or 3-12 membered heterocyclene, each optionally substituted with one or more R¹⁶;
each R¹³ is independently selected from the group consisting of H, D, C₁-C₆ alkyl, C₃-C₆ cycloalkyl and 3-6-membered heterocyclyl, wherein each said C₁-C₆ alkyl is optionally substituted with one or more R^{17A}, and each said C₃-C₆ cycloalkyl and 3-6-membered heterocyclyl is optionally substituted with one or more R^{17B};
each R¹⁴ is independently selected from the group consisting of H, D, F, OH, NH₂, C₁-C₆ alkyl, C₃-C₆ cycloalkyl, C₁-C₆ alkoxy, C₃-C₆ cycloalkoxy, C₁-C₆ alkylamino, C₃-C₆ cycloalkylamino, and 3-6 membered heterocyclyl, wherein each said C₁-C₆ alkyl, C₁-C₆ alkoxy, C₁-C₆ alkylamino and (C₁—C alkyl)₂amino is optionally substituted with one or more R^{18A}, and each said C₃-C₆ cycloalkyl and 3-6 membered heterocyclyl is optionally substituted with one or more R^{18B}; or two R¹⁴ together with the atom(s) to which they are attached optionally form a C₃-C₆ carbocyclyl or 3-6 membered heterocyclyl;
each R¹⁵ is independently selected from the group consisting of H, D, F, CN, OH, NH₂, C₁-C₆alkyl, C₃-C₆ cycloalkyl, C₁-C₆ alkoxy, C₃-C₆ cycloalkoxy, C₁-C₆ alkylamino, (C₁-C₆ alkyl)₂amino, C₃-C₆ cycloalkylamino and oxo, wherein each said C₁-C₆ alkyl, C₁-C₆ alkoxy, C₁-C₆ alkylamino and (C₁-C₆ alkyl)₂amino is optionally substituted with one or more R^{19A}, and each said C₃-C₆ cycloalkyl, C₃-C₆ cycloalkoxy, C₃-C₆ cycloalkylamino, and 3-6 membered heterocyclyl is optionally substituted with one or more R^{19B};
each R^{17A}, R^{18A} and R^{19A} is independently selected from the group consisting of D, F, OH, C₁-C₄ alkoxy, oxo, NH₂, NH(C₁-C₄ alkyl) and N(C₁-C₄ alkyl)₂; and
each R¹⁶, R^{17B}, R^{18B} and R^{19B} is independently selected from the group consisting of D, F, OH, C₁-C₄ alkyl, C₁-C₄ fluoroalkyl, C₁-C₄ alkoxy, oxo, NH₂, NH(C₁-C₄ alkyl) and N(C₁-C₄ alkyl)₂.

In some embodiments of Formula (J), x is an integer from 1 to 20. In some embodiments of Formula (J), x is an integer from 1 to 15. In some embodiments of Formula (J), x is an integer from 1 to 10. In some embodiments of Formula (J), x is an integer from 1 to 5.

In some embodiments, where x is an integer from 1 to 10, Formula (J) may be represented as:

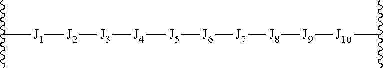

wherein each unit J₁, J₂, J₃, J₄, J₅, J₆, J₇, J₈, J₉ and J₁₀ is independently defined as for -(J)-.

In some embodiments, where x is an integer from 1 to 5, Formula (J) may be represented as:

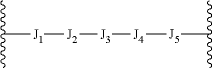

wherein each unit J₁, J₂, J₃, J₄ and J₅ is independently defined as for -(J)-.

In some embodiments, where x is an integer from 1 to 3, Formula (J) may be represented as:

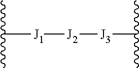

wherein each unit J₁, J₂ and J₃ is independently defined as for -(J)-.

Similar representations may be used for other integer ranges of x in Formula (J).

In some embodiments of Formula (J), one or more groups -(J)- are sequentially arranged in a pattern of repeating polyethylene glycol (PEG) units comprising —(CH₂—CH₂—O)—. In some embodiments of Formula (J), one or more groups -(J)- are sequentially arranged in a pattern of repeating carboxamide units comprising —C(O)—N(R¹³)— or —C(O)—N(R¹³)—(C(R¹⁴)₂)₁₋₃)—. In some embodiments of Formula (J), one or more (J)- of -(J)ₓ- are sequentially arranged in a pattern of alternating polyethylene glycol (PEG) units comprising —(CH₂—CH₂—O)— and carboxamide units comprising —C(O)—N(R¹³).

In some preferred embodiments of any of Formulae (I)-(XIII), L¹ is a bivalent linker of Formula (L-1), wherein A_{L} and B_{L} are defined as in Table 3.

TABLE 3
Exemplary Linkers of Formula (L-1)

| A_{L}= | R_{L}^{a} | R_{L}^{b} | B_{L} = R_{L}^{a} = R_{L}^{r} |
|---|---|---|---|
| R_{L}^{a}C(O)NHR_{L}^{b} | C₁-C₄ alkylene | C₁-C₄ alkylene | R_{L}^{r} = heteroaryl |
| R_{L}^{a}C(O)NHR_{L}^{b} | C₁-C₄ alkylene | C₁-C₄ alkylene | R_{L}^{r} = cycloalkyl |
| R_{L}^{a}C(O)NHR_{L}^{b} | C₁-C₄ alkylene | C₁-C₄ alkylene | R_{L}^{r} = heterocyclyl |
| R_{L}^{a}C(O)NHR_{L}^{b} | (C₁-C₄ alkylene)-R_{L}^{r}; | C₁-C₄ alkylene | R_{L}^{r} = cycloalkyl |
| | R_{L}^{r} = cycloalkyl | | |
| R_{L}^{a}C(O)NHR_{L}^{b} | C₁-C₄ alkylene | bond | R_{L}^{r} = cycloalkyl |
| R_{L}^{a}C(O)N(R_{L}¹)R_{L}^{b} | C₁-C₄ alkylene | bond | R_{L}^{r} = cycloalkyl |
| R_{L}^{a}C(O)N(R_{L}¹)R_{L}^{b} | C₁-C₄ alkylene | C₁-C₄ alkylene | R_{L}^{r} = cycloalkyl |
| R_{L}^{a}C(O)NHR_{L}^{b} | bond | C₁-C₄ alkylene | R_{L}^{r} = cycloalkyl |
| R_{L}^{a}C(O)NHR_{L}^{b} | bond | bond | R_{L}^{r} = cycloalkyl |
| R_{L}^{a}C(O)NHR_{L}^{b} | R_{L}^{r}; | bond | R_{L}^{r} = cycloalkyl |
| | R_{L}^{r} = heterocyclyl | | |
| R_{L}^{a}C(O)NHR_{L}^{b} | R_{L}^{r}; | C₁-C₄ alkylene | R_{L}^{r} = cycloalkyl |
| | R_{L}^{r} = heterocyclyl | | |
| R_{L}^{a}C(O)NHR_{L}^{b} | R_{L}^{r}; | bond | R_{L}^{r} = heterocyclyl |
| | R_{L}^{r} = heterocyclyl | | |
| R_{L}^{a}C(O)NHR_{L}^{b} | R_{L}^{r}; | C₁-C₄ alkylene | R_{L}^{r} = heterocyclyl |
| | R_{L}^{r} = heterocyclyl | | |
| R_{L}^{a}C(O)NHR_{L}^{b} | R_{L}^{r}; | bond | R_{L}^{r} = cycloalkyl |
| | R_{L}^{r} = cycloalkyl | | |
| R_{L}^{a}C(O)NHR_{L}^{b} | R_{L}^{r}; | bond | R_{L}^{r} = heterocyclyl |
| | R_{L}^{r} = cycloalkyl | | |
| R_{L}^{a}N(R_{L}¹)C(O)R_{L}^{b} | C₁-C₄ alkylene | bond | R_{L}^{r} = cycloalkyl |
| R_{L}^{a}N(R_{L}¹)C(O)R_{L}^{b} | C₁-C₄ alkylene | C₁-C₄ alkylene | R_{L}^{r} = cycloalkyl |
| R_{L}^{a}N(R_{L}¹)C(O)R_{L}^{b} | bond | C₁-C₄ alkylene | R_{L}^{r} = cycloalkyl |
| R_{L}^{a}N(R_{L}¹)C(O)R_{L}^{b} | bond | bond | R_{L}^{r} = cycloalkyl |
| R_{L}^{a}N(R_{L}¹)C(O)R_{L}^{b} | bond | bond | R_{L}^{r} = heterocyclyl |
| R_{L}^{a}N(R_{L}¹)C(O)R_{L}^{b} | bond | C₁-C₄ alkylene | R_{L}^{r} = heterocyclyl |
| R_{L}^{a}C(O)R_{L}^{b} | C₁-C₄ alkylene | bond | R_{L}^{r} = cycloalkyl |
| R_{L}^{a}C(O)R_{L}^{b} | C₁-C₄ alkylene | bond | R_{L}^{r} = heterocyclyl |
| R_{L}^{a}C(O)R_{L}^{b} | (C₁-C₄ alkylene)-R_{L}^{r}; | bond | R_{L}^{r} = heterocyclyl |
| | R_{L}^{r} = cycloalkyl | | |
| R_{L}^{a}C(O)R_{L}^{b} | bond | C₁-C₄ alkylene | R_{L}^{r} = cycloalkyl |
| R_{L}^{a}C(O)R_{L}^{b} | bond | C₁-C₄ alkylene | R_{L}^{r} = heterocyclyl |
| R_{L}^{a}C(O)R_{L}^{b} | bond | bond | R_{L}^{r} = heterocyclyl |
| RL^{a}C(O)R_{L}^{b} | bond | bond | R_{L}^{r} = cycloalkyl |
| R_{L}^{a}C(O)R_{L}^{b} | R_{L}^{r}; | bond | R_{L}^{r} = cycloalkyl |
| | R_{L}^{r} = heterocyclyl | | |
| R_{L}^{a}C(O)R_{L}^{b} | R_{L}^{r}; | bond | R_{L}^{r} = heterocyclyl |
| | R_{L}^{r} = heterocyclyl | | |
| R_{L}^{a}C(O)R_{L}^{b} | (C₁-C₈ alkylene)-R_{L}^{r}; | bond | R_{L}^{r} = heterocyclyl |
| | R_{L}^{r} = cycloalkyl | | |
| R_{L}^{a}-R_{L}^{b} | C₁-C₄ alkylene | C₁-C₄ alkylene | R_{L}^{r} = cycloalkyl |
| R_{L}^{a}-R_{L}^{b} | (C₁-C₄ alkylene)-R_{L}^{r}; | C₁-C₄ alkylene | R_{L}^{r} = cycloalkyl |
| | R_{L}^{r} = heteroaryl | | |
| R_{L}^{a}-R_{L}^{b} | (C₁-C₄ alkylene)-R_{L}^{r}; | C₁-C₄ alkylene | R_{L}^{r} = cycloalkyl |
| | R_{L}^{r} = heterocyclyl | | |
| R_{L}^{a} | (C₁-C₄ alkylene)-R_{L}^{r}; | — | R_{L}^{r} = cycloalkyl |
| | R_{L}^{r} = heteroaryl | | |
| R_{L}^{a} | (C₁-C₄ alkylene)-R_{L}^{r}; | — | R_{L}^{r} = cycloalkyl |
| | R_{L}^{r} = heterocyclyl | | |
| R_{L}^{a}NHR_{L}^{b} | C₁-C₄ alkylene | C₁-C₄ alkylene | R_{L}^{r} = cycloalkyl |
| R_{L}^{a}NHR_{L}^{b} | C₁-C₈ alkylene | bond | R_{L}^{r} = cycloalkyl |
| R_{L}^{a}NHR_{L}^{b} | bond | C₁-C₈ alkylene | R_{L}^{r} = cycloalkyl |
| A_{L}= | R_{L}^{a} | R_{L}^{b} | B_{L} = R_{L}^{a}C(O)R_{L}^{b} |
| R_{L}^{a}NHR_{L}^{b} | bond | C₁-C₄ alkylene | R_{L}^{a}C(O)R_{L}^{b}, |
| | | | R_{L}^{a} = bond, R_{L}^{b} = |
| | | | R_{L}^{r} = heterocyclyl |
| R_{L}^{a}N(R_{L}¹)R_{L}^{b} | bond | C₁-C₄ alkylene | R_{L}^{a}C(O)R_{L}^{b}, |
| | | | R_{L}^{a} = bond, R_{L}^{b} = |
| | | | R_{L}^{r} = heterocyclyl |
| A_{L}= | R_{L}^{a} | R_{L}^{b} | B_{L} = R_{L}^{a}C(O)NHR_{L}^{b} |
| R_{L}^{a}NHR_{L}^{b} | bond | C₁-C₄ alkylene | R_{L}^{a} = bond, R_{L}^{b} = |
| | | | R_{L}^{r} = cycloalkyl |
| A_{L}= | R_{L}^{a} | R_{L}^{b} | B_{L} = R_{L}^{a} = |
| | | | R_{L}^{r}-(C₁-C₄ |
| | | | alkylene) |
| R_{L}^{a}C(O)NHR_{L}^{b} | C₁-C₄ alkylene | bond | R_{L}^{r} = heterocyclyl |
| R_{L}^{a}C(O)NHR_{L}^{b} | C₁-C₄ alkylene | C₁-C₄ alkylene | R_{L}^{r} = cycloalkyl |
| R_{L}^{a}C(O)R_{L}^{b} | C₁-C₄ alkylene | bond | R_{L}^{r} = heterocyclyl |
| R_{L}^{a}C(O)R_{L}^{b} | bond | C₁-C₄ alkylene | R_{L}^{r} = cycloalkyl |
| A_{L}= | R_{L}^{a} | R_{L}^{b} | B_{L} = R_{L}^{a}OR_{L}^{b} |
| R_{L}^{a}C(O)R_{L}^{b} | bond | C₁-C₄ alkylene | R_{L}^{a} = bond |
| | | | R_{L}^{b} = (C₁-C₈ |
| | | | alkylene)-R_{L}^{r} |
| | | | R_{L}^{a} = cycloalkyl |
| R_{L}^{a}C(O)R_{L}^{b} | bond | C₁-C₄ alkylene | R_{L}^{a} = bond |
| | | | R_{L}^{b} = R_{L}^{r}-(C₁-C₈ |
| | | | alkylene) |
| | | | R_{L}^{r} = cycloalkyl |
| R_{L}^{a}C(O)R_{L}^{b} | bond | C₁-C₄ alkylene | R_{L}^{a} = bond |
| | | | R_{L}^{b} = R_{L}^{r} |
| | | | R_{L}^{r} = cycloalkyl |
| A_{L}= | R_{L}^{a} | R_{L}^{b} | B_{L} = R_{L}^{a}NHR_{L}^{b} |
| R_{L}^{a}C(O)R_{L}^{b} | bond | C₁-C₄ alkylene | R_{L}^{a} = bond |
| | | | R_{L}^{b} = R_{L}^{r} |
| | | | R_{L}^{r} = cycloalkyl |
| A_{L}= | R_{L}^{a} | R_{L}^{b} | B_{L} = bond |
| R_{L}^{a}C(O)NHR_{L}^{b} | C₁-C₄ alkylene | C₁-C₄ alkylene | — |
| R_{L}^{a}C(O)NHR_{L}^{b} | C₁-C₄ alkylene | R_{L}^{r}-(C₁-C₄ | — |
| | | alkylene); | |
| | | R_{L}^{r} = cycloalkyl | |
| R_{L}^{a}C(O)NHR_{L}^{b} | bond | (C₁-C₄ alkylene)-R_{L}^{r}- | — |
| | | (C₁-C₄ alkylene); | |
| | | R_{L}^{r} = cycloalkyl | |
| R_{L}^{a}NHC(O)R_{L}^{b} | C₁-C₄ alkylene | C₁-C₄ alkylene | — |
| R_{L}^{a}C(O)R_{L}^{b} | C₁-C₄ alkylene | bond | — |
| R_{L}^{a}C(O)R_{L}^{b} | C₁-C₄ alkylene | R_{L}^{r}-(C₁-C₄ | — |
| | | alkylene); | |
| | | R_{L}^{r} = heterocycle | |
| R_{L}^{a}C(O)R_{L}^{b} | R_{L}^{a} = R_{L}^{r}, | R_{L}^{b} = R_{L}^{r}, | — |
| | R_{L}^{r} = heterocyclyl | R_{L}^{r} = cycloalkyl | |
| R_{L}^{a}C(O)R_{L}^{b} | R_{L}^{a} = R_{L}^{r}, | R_{L}^{b} = R_{L}^{r}, | — |
| | R_{L}^{r} = cycloalkyl | R_{L}^{r} = heterocyclyl | |
| R_{L}^{a}C(O)R_{L}^{b} | R_{L}^{a} = R_{L}^{r}, | R_{L}^{b} = R_{L}^{r}, | — |
| | R_{L}^{r} = heterocyclyl | R_{L}^{r} = heterocyclyl | |
| R_{L}^{a}-R_{L}^{b} | (C₁-C₄ alkylene)-R_{L}^{r}; | C₁-C₄ alkylene | — |
| | R_{L}^{r} = heteroaryl | | |
| R_{L}^{a}NHR_{L}^{b} | C₁-C₄ alkylene | C₁-C₄ alkylene | — |

In some embodiments of any of Formulae (I)-(XIII), L¹ is a bivalent linker selected from the group consisting of:

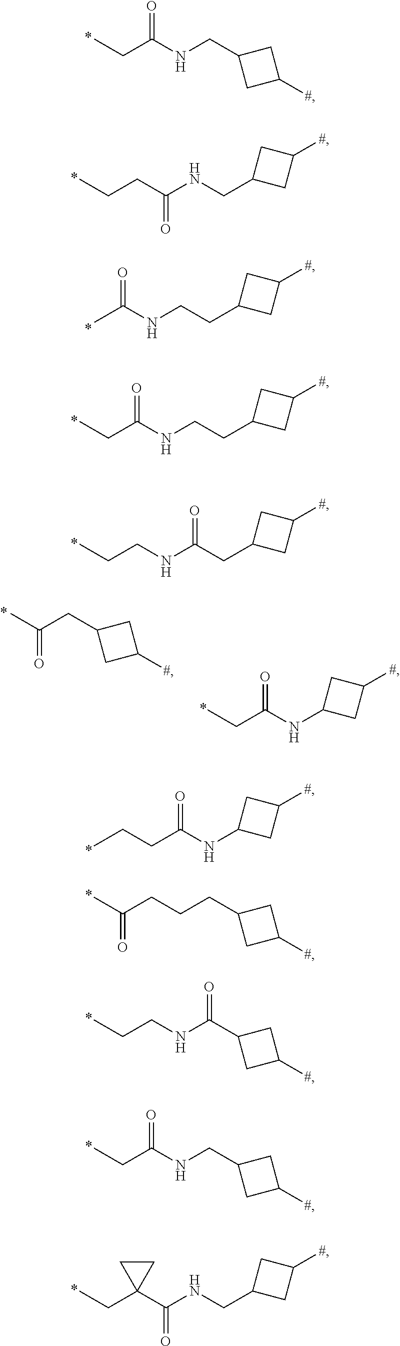

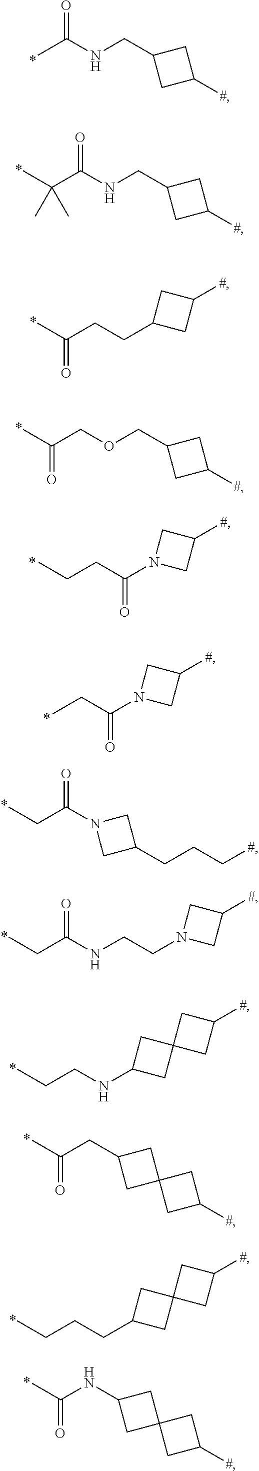

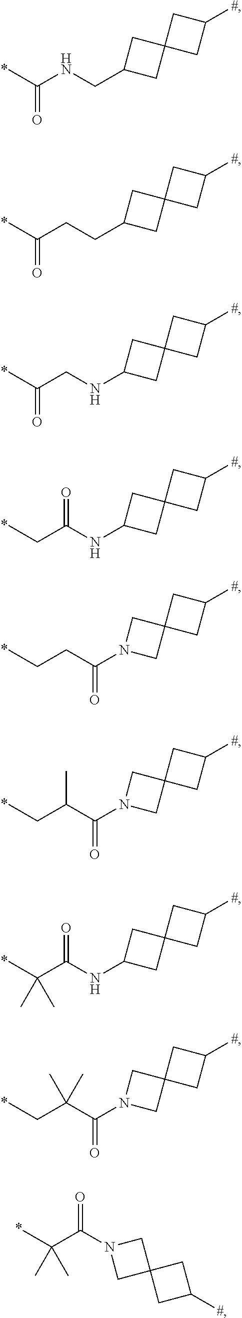

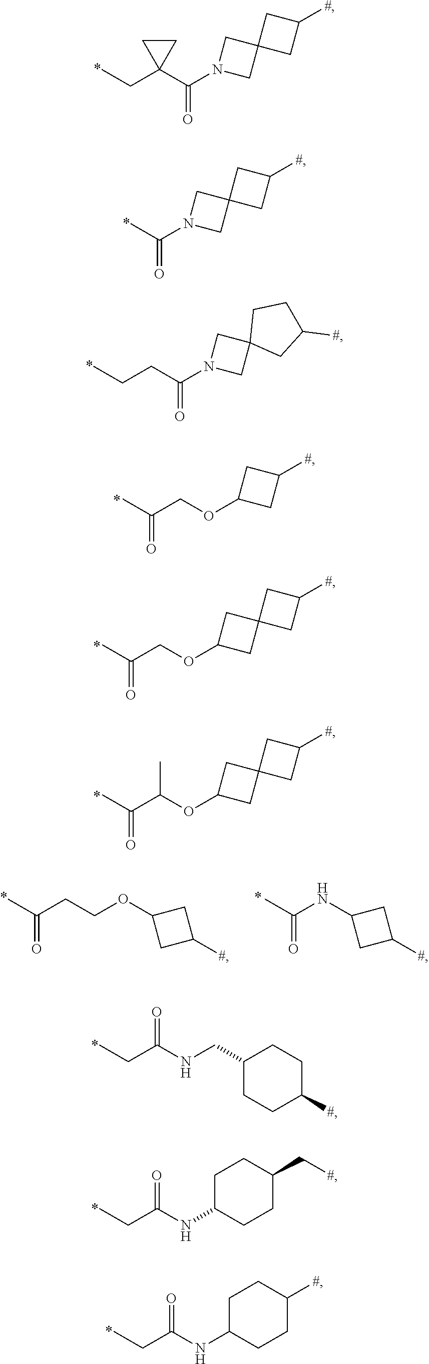

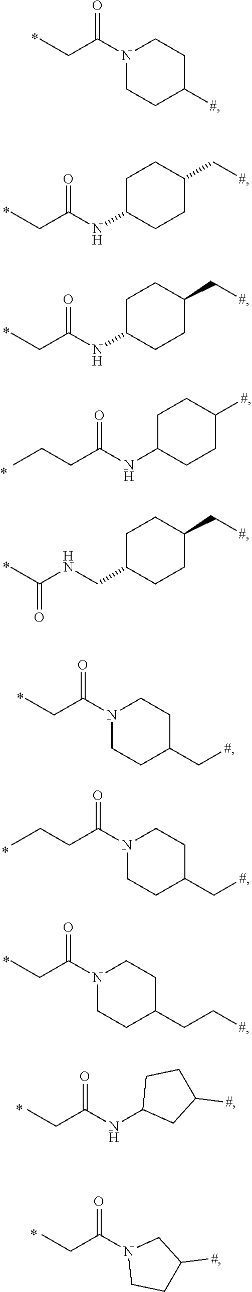

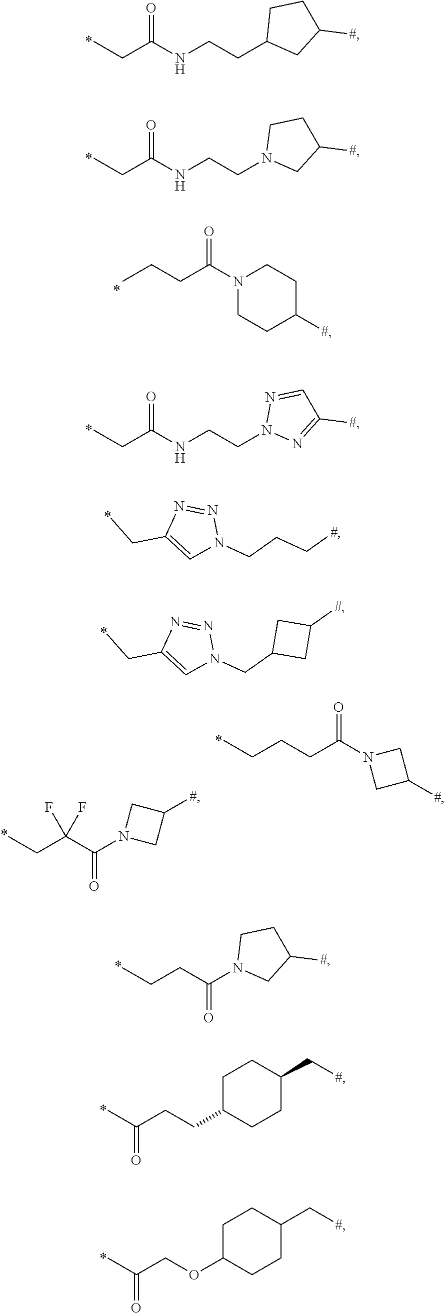

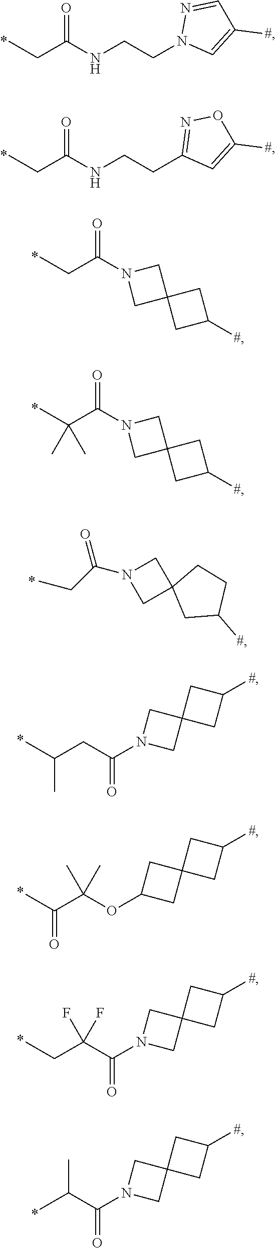

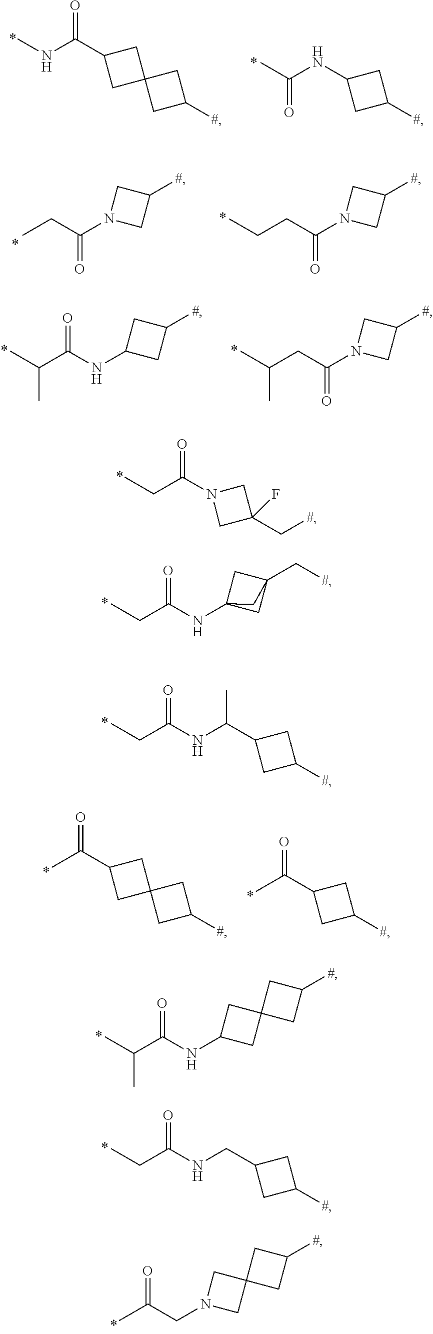

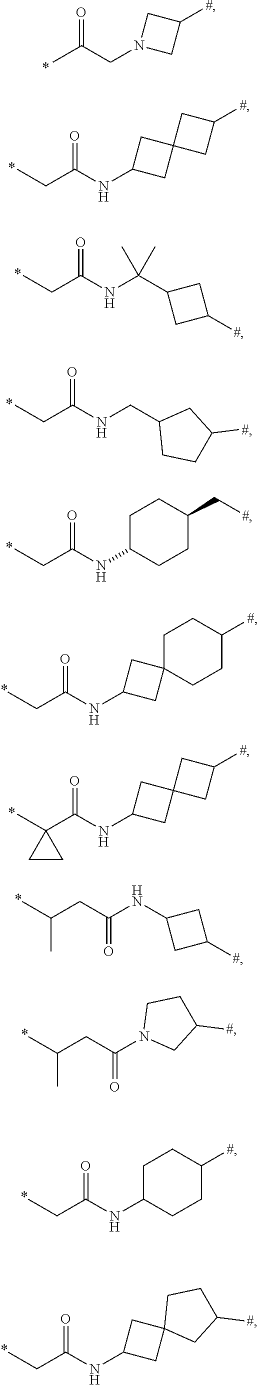

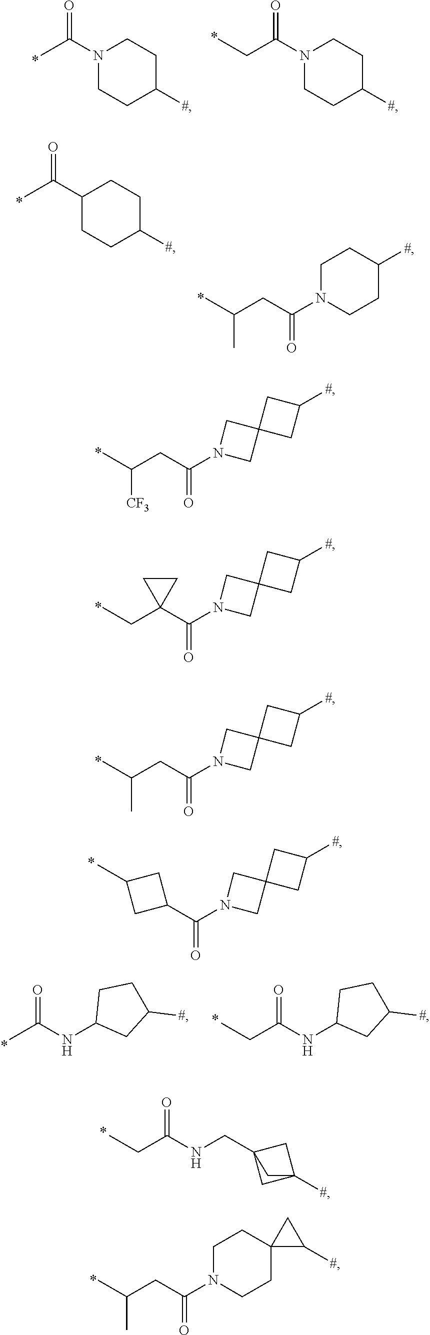

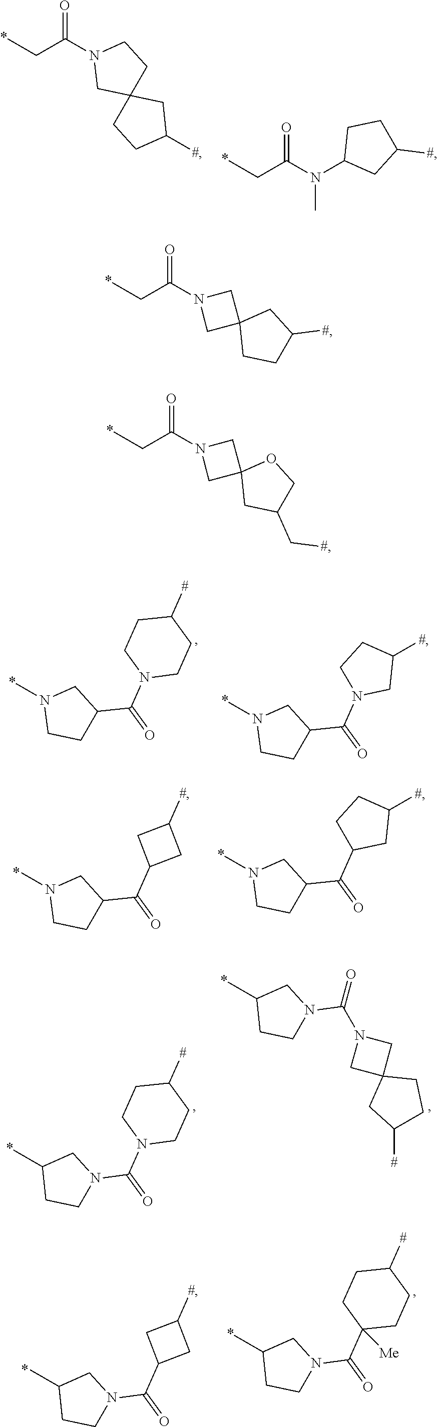

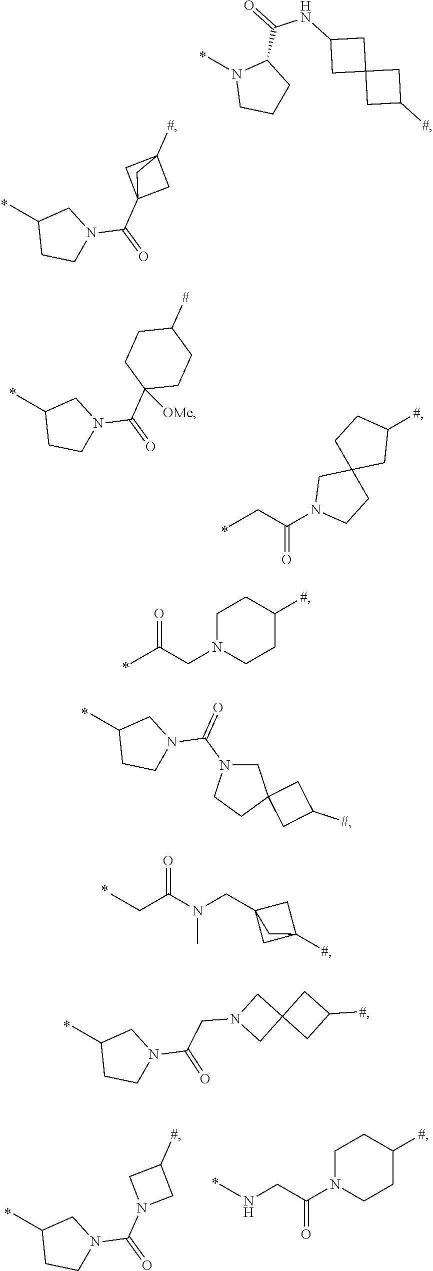

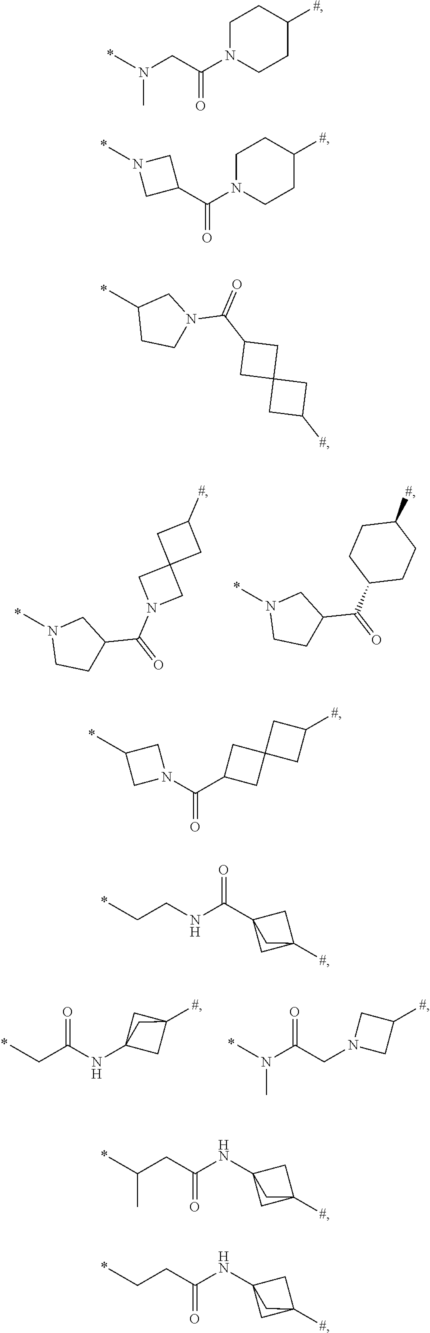

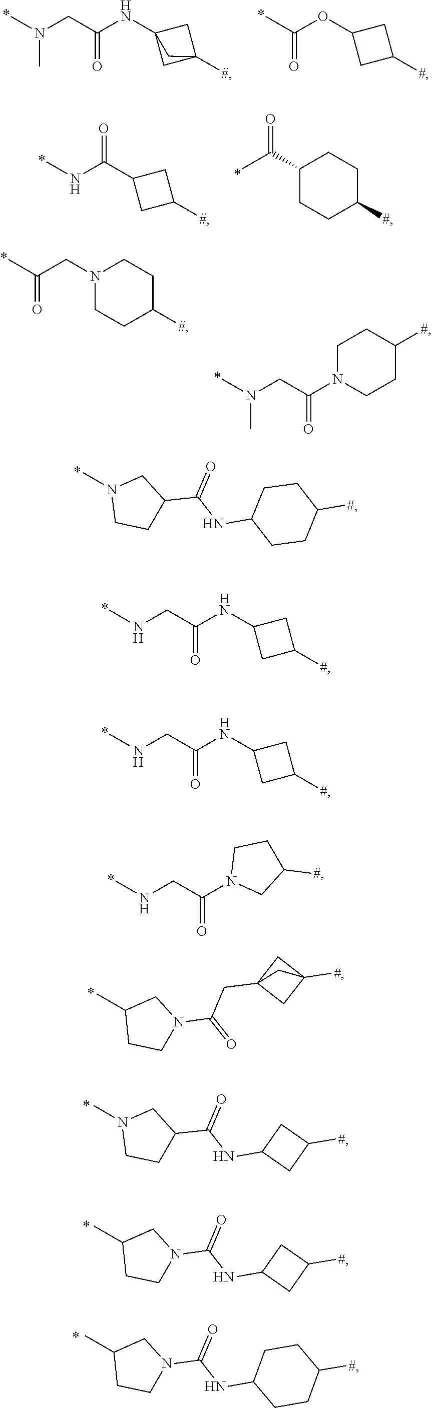

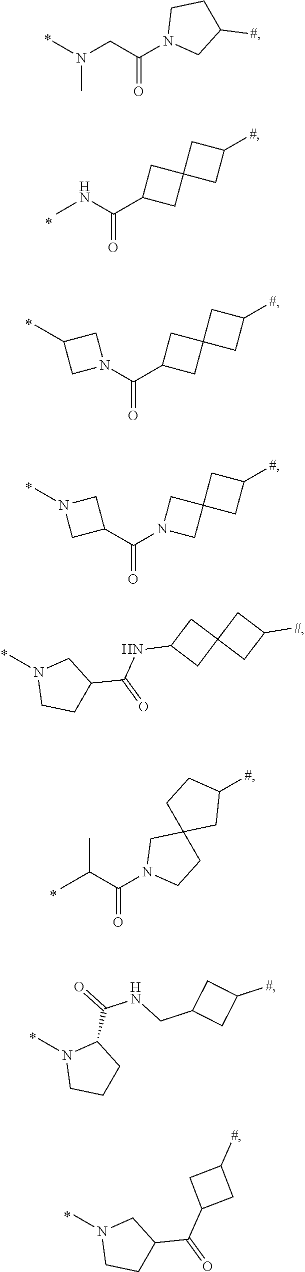

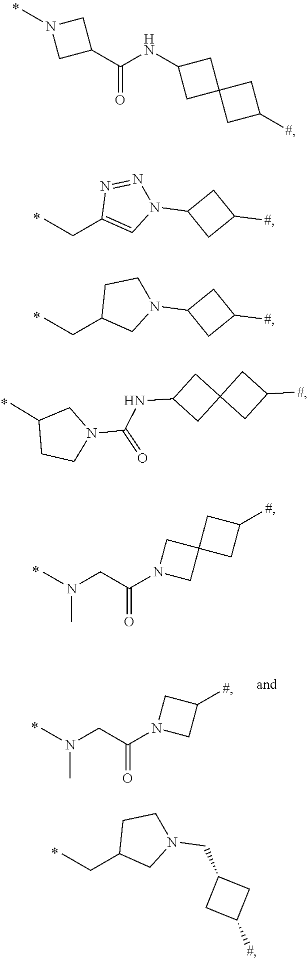

where #is the point of attachment to L² and * is the point of attachment to A in any of Formulae (I)-(XII); or #is the point of attachment to L² and * is the point of attachment to Z² or G in Formula (XIII).

In some embodiments of any of Formulae (I)-(XIII), L¹ is a bivalent linker selected from the group consisting of:

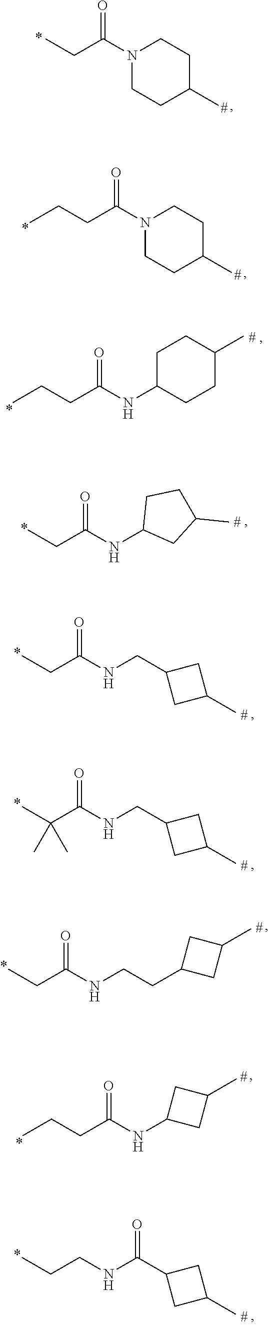

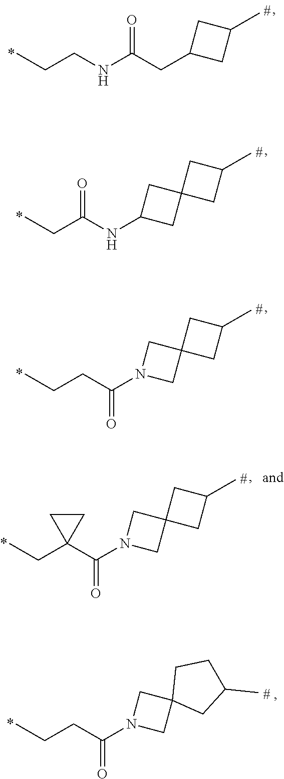

where #is the point of attachment to L² and * is the point of attachment to A in any of Formulae (I)-(XII); or #is the point of attachment to L² and * is the point of attachment to Z² or G in Formula (XIII).

Certain exemplary DDB1 binding compounds and intermediates have the structures shown in Table 1A and Table 1B.

TABLE 1A
Representative DDB1 binding compounds and intermediates

| Cpd. No. | Structure | Chemical Name |
|---|---|---|
| B1-1 | 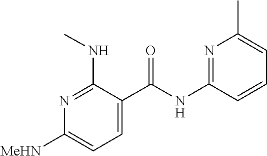 [SMILES: CNc1ccc(C(=O)Nc2cccc(C)n2)c(NC)n1] | 2,6-bis(methylamino)-N-(6- methylpyridin-2-yl)nicotinamide |
| B1-2 | 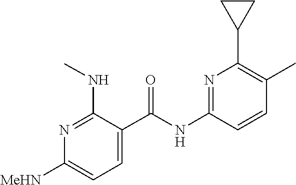 [SMILES: CNc1ccc(C(=O)Nc2ccc(C)c(C3CC3)n2)c(NC)n1] | N-(6-cyclopropyl-5-methylpyridin-2- yl)-2,6- bis(methylamino)nicotinamide |
| B1-3 | 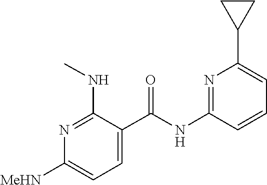 [SMILES: CNc1ccc(C(=O)Nc2cccc(C3CC3)n2)c(NC)n1] | N-(6-cyclopropylpyridin-2-yl)-2,6- bis(methylamino)nicotinamide |
| B1-4 | 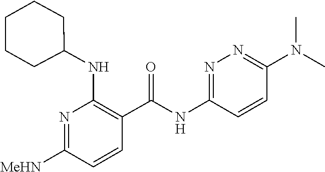 [SMILES: CNc1ccc(C(=O)Nc2ccc(N(C)C)nn2)c(NC2CCCCC2)n1] | 2-(cyclohexylamino)-N-(6- (dimethylamino)pyridazin-3-yl)-6- (methylamino)nicotinamide |
| B1-5 | 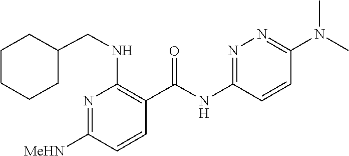 [SMILES: CNc1ccc(C(=O)Nc2ccc(N(C)C)nn2)c(NCC2CCCCC2)n1] | 2-((cyclohexylmethyl)amino)-N-(6- (dimethylamino)pyridazin-3-yl)-6- (methylamino)nicotinamide |
| B1-6 | 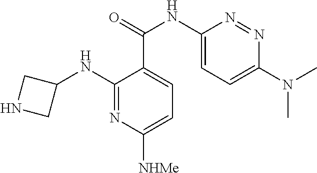 [SMILES: CNc1ccc(C(=O)Nc2ccc(N(C)C)nn2)c(NC2CNC2)n1] | 2-(azetidin-3-ylamino)-N-(6- (dimethylamino)pyridazin-3-yl)-6- (methylamino)nicotinamide |
| B1-7 | 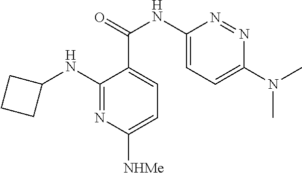 [SMILES: CNc1ccc(C(=O)Nc2ccc(N(C)C)nn2)c(NC2CCC2)n1] | 2-(cyclobutylamino)-N-(6- (dimethylamino)pyridazin-3-yl)-6- (methylamino)nicotinamide |
| B1-8 | 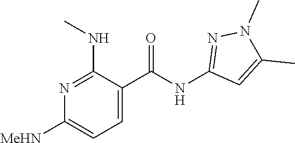 [SMILES: CNc1ccc(C(=O)Nc2cc(C)n(C)n2)c(NC)n1] | N-(1,5-dimethyl-1H-pyrazol-3-yl)- 2,6-bis(methylamino)nicotinamide |
| B1-9 | 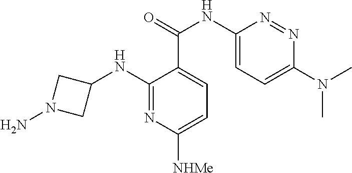 [SMILES: CNc1ccc(C(=O)Nc2ccc(N(C)C)nn2)c(NC2CN(N)C2)n1] | 2-((3-aminocyclobutyl)amino)-N-(6- (dimethylamino)pyridazin-3-yl)-6- (methylamino)nicotinamide |
| B1-10 | 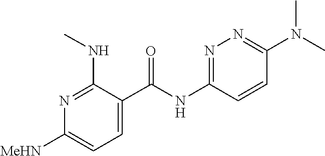 [SMILES: CNc1ccc(C(=O)Nc2ccc(N(C)C)nn2)c(NC)n1] | N-(6-(dimethylamino)pyridazin-3- yl)-2,6- bis(methylamino)nicotinamide |
| B1-11 | 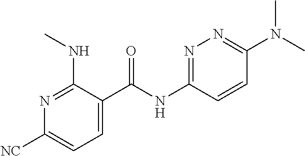 [SMILES: CNc1nc(C#N)ccc1C(=O)Nc1ccc(N(C)C)nn1] | 6-cyano-N-(6- (dimethylamino)pyridazin-3-yl)-2- (methylamino)nicotinamide |
| B1-12 | 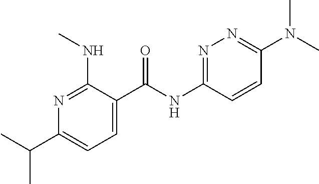 [SMILES: CNc1nc(C(C)C)ccc1C(=O)Nc1ccc(N(C)C)nn1] | N-(6-(dimethylamino)pyridazin-3- yl)-6-isopropyl-2- (methylamino)nicotinamide |
| B1-13 | 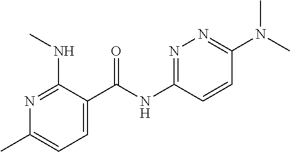 | N-(6-(dimethylamino)pyridazin-3- yl)-6-methyl-2- (methylamino)nicotinamide |
| B1-14 | 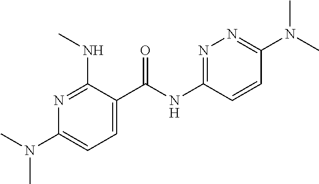 | 6-(dimethylamino)-N-(6- (dimethylamino)pyridazin-3-yl)-2- (methylamino)nicotinamide |
| B1-15 | 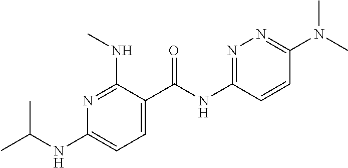 [SMILES: CNc1nc(NC(C)C)ccc1C(=O)Nc1ccc(N(C)C)nn1] | N-(6-(dimethylamino)pyridazin-3- yl)-6-(isopropylamino)-2- (methylamino)nicotinamide |
| B1-16 | 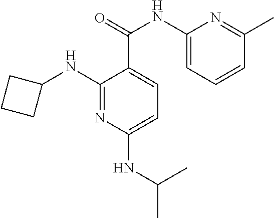 [SMILES: Cc1cccc(NC(=O)c2ccc(NC(C)C)nc2NC2CCC2)n1] | 2-(cyclobutylamino)-6- (isopropylamino)-N-(6- methylpyridin-2-yl)nicotinamide |
| B1-17 | 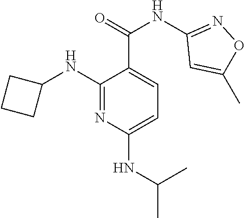 [SMILES: Cc1cc(NC(=O)c2ccc(NC(C)C)nc2NC2CCC2)no1] | 2-(cyclobutylamino)-6- (isopropylamino)-N-(5- methylisoxazol-3-yl)nicotinamide |
| B1-18 | 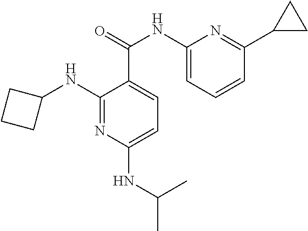 [SMILES: CC(C)Nc1ccc(C(=O)Nc2cccc(C3CC3)n2)c(NC2CCC2)n1] | 2-(cyclobutylamino)-N-(6- cyclopropylpyridin-2-yl)-6- (isopropylamino)nicotinamide |
| B1-19 | 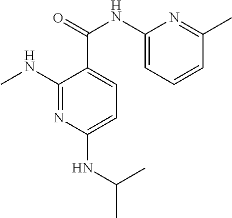 | 6-(isopropylamino)-2- (methylamino)-N-(6-methylpyridin- 2-yl)nicotinamide |
| B1-20 | 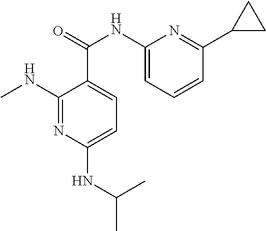 [SMILES: CNc1nc(NC(C)C)ccc1C(=O)Nc1cccc(C2CC2)n1] | N-(6-cyclopropylpyridin-2-yl)-6- (isopropylamino)-2- (methylamino)nicotinamide |
| B1-21 | 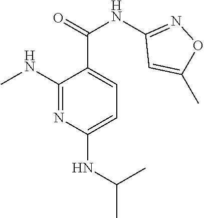 | 6-(isopropylamino)-2- (methylamino)-N-(5-methylisoxazol- 3-yl)nicotinamide |
| B1-22 | 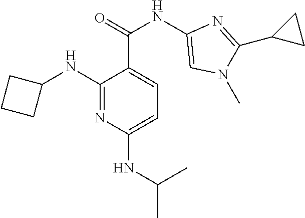 [SMILES: CC(C)Nc1ccc(C(=O)Nc2cn(C)c(C3CC3)n2)c(NC2CCC2)n1] | 2-(cyclobutylamino)-N-(2- cyclopropyl-1-methyl-1H-imidazol- 4-yl)-6- (isopropylamino)nicotinamide |
| B1-23 | 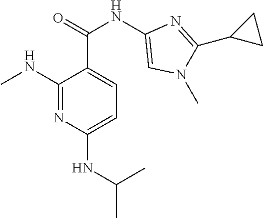 [SMILES: CNc1nc(NC(C)C)ccc1C(=O)Nc1cn(C)c(C2CC2)n1] | N-(2-cyclopropyl-1-methyl-1H- imidazol-4-yl)-6-(isopropylamino)- 2-(methylamino)nicotinamide |
| B1-24 | 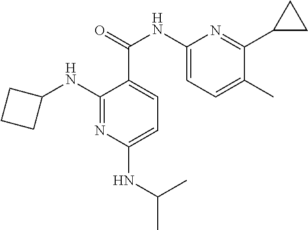 [SMILES: Cc1ccc(NC(=O)c2ccc(NC(C)C)nc2NC2CCC2)nc1C1CC1] | 2-(cyclobutylamino)-N-(6- cyclopropyl-5-methylpyridin-2-yl)- 6-(isopropylamino)nicotinamide |
| B1-25 | 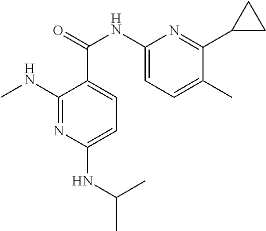 [SMILES: CNc1nc(NC(C)C)ccc1C(=O)Nc1ccc(C)c(C2CC2)n1] | N-(6-cyclopropyl-5-methylpyridin-2- yl)-6-(isopropylamino)-2- (methylamino)nicotinamide |

TABLE 1B
Representative DDB1 binding compounds and intermediates

| Cpd. No. | Structure | Chemical Name |
|---|---|---|
| BL1-1 | 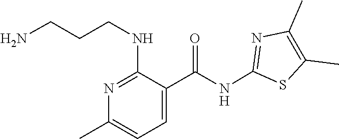 [SMILES: Cc1ccc(C(=O)Nc2nc(C)c(C)s2)c(NCCCN)n1] | 2-((3-aminopropyl)amino)-N-(4,5- dimethylthiazol-2-yl)-6- methylnicotinamide |
| BL1-2 | 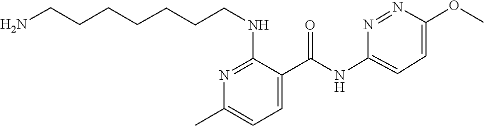 [SMILES: COc1ccc(NC(=O)c2ccc(C)nc2NCCCCCCCN)nn1] | 2-((7-aminoheptyl)amino)-N-(6- methoxypyridazin-3-yl)-6- methylnicotinamide |
| BL1-3 | 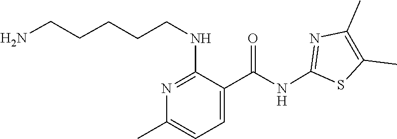 [SMILES: Cc1ccc(C(=O)Nc2nc(C)c(C)s2)c(NCCCCCN)n1] | 2-((5-aminopentyl)amino)-N-(4,5- dimethylthiazol-2-yl)-6- methylnicotinamide |
| BL1-4 | 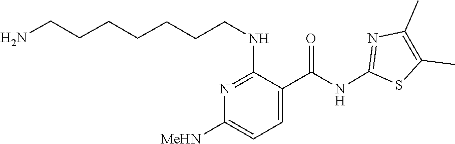 [SMILES: CNc1ccc(C(=O)Nc2nc(C)c(C)s2)c(NCCCCCCCN)n1] | 2-((7-aminoheptyl)amino)-N-(4,5- dimethylthiazol-2-yl)-6- (methylamino)nicotinamide |
| BL1-5 | 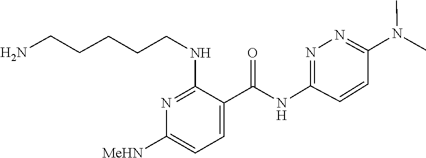 [SMILES: CNc1ccc(C(=O)Nc2ccc(N(C)C)nn2)c(NCCCCCN)n1] | 2-((5-aminopentyl)amino)-N-(6- (dimethylamino)pyridazin-3-yl)-6- (methylamino)nicotinamide |
| BL1-6 | 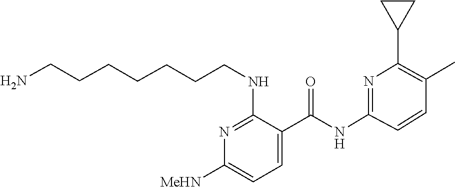 [SMILES: CNc1ccc(C(=O)Nc2ccc(C)c(C3CC3)n2)c(NCCCCCCCN)n1] | 2-((7-aminoheptyl)amino)-N-(6- cyclopropyl-5-methylpyridin-2-yl)- 6-(methylamino)nicotinamide |
| BL1-7 | 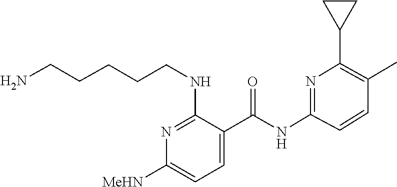 [SMILES: CNc1ccc(C(=O)Nc2ccc(C)c(C3CC3)n2)c(NCCCCCN)n1] | 2-((5-aminopentyl)amino)-N-(6- cyclopropyl-5-methylpyridin-2-yl)- 6-(methylamino)nicotinamide |
| BL1-8 | 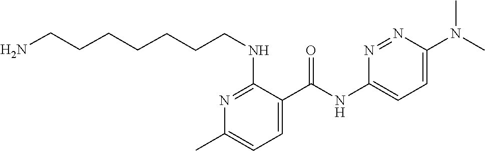 [SMILES: Cc1ccc(C(=O)Nc2ccc(N(C)C)nn2)c(NCCCCCCCN)n1] | 2-((7-aminoheptyl)amino)-N-(6- (dimethylamino)pyridazin-3-yl)-6- methylnicotinamide |
| BL1-9 | 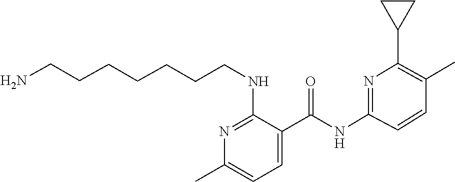 [SMILES: Cc1ccc(C(=O)Nc2ccc(C)c(C3CC3)n2)c(NCCCCCCCN)n1] | 2-((7-aminoheptyl)amino)-N-(6- cyclopropyl-5-methylpyridin-2-yl)- 6-methylnicotinamide |
| BL1-10 | 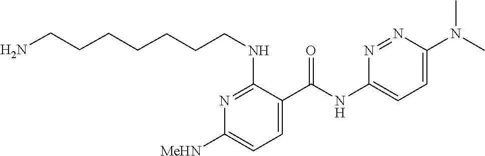 [SMILES: CNc1ccc(C(=O)Nc2ccc(N(C)C)nn2)c(NCCCCCCCN)n1] | 2-((7-aminoheptyl)amino)-N-(6- (dimethylamino)pyridazin-3-yl)-6- (methylamino)nicotinamide |
| BL1-11 | 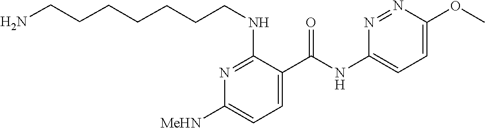 [SMILES: CNc1ccc(C(=O)Nc2ccc(OC)nn2)c(NCCCCCCCN)n1] | 2-((7-aminoheptyl)amino)-N-(6- methoxypyridazin-3-yl)-6- (methylamino)nicotinamide |
| BL1-12 | 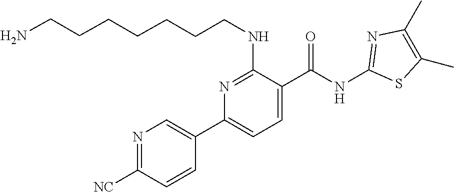 [SMILES: Cc1nc(NC(=O)c2ccc(-c3ccc(C#N)nc3)nc2NCCCCCCCN)sc1C] | 6-((7-aminoheptyl)amino)-6′-cyano- N-(4,5-dimethylthiazol-2-yl)-[2,3′- bipyridine]-5-carboxamide |
| BL1-13 | 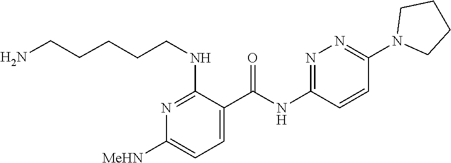 [SMILES: CNc1ccc(C(=O)Nc2ccc(N3CCCC3)nn2)c(NCCCCCN)n1] | 2-((5-aminopentyl)amino)-6- (methylamino)-N-(6-(pyrrolidin-1- yl)pyridazin-3-yl)nicotinamide |
| BL1-14 | 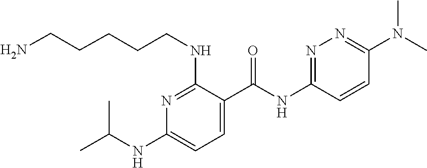 [SMILES: CC(C)Nc1ccc(C(=O)Nc2ccc(N(C)C)nn2)c(NCCCCCN)n1] | 2-((5-aminopentyl)amino)-N-(6- (dimethylamino)pyridazin-3-yl)-6- (isopropylamino)nicotinamide |
| BL1-15 | 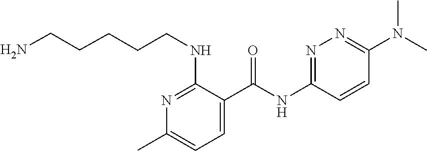 [SMILES: Cc1ccc(C(=O)Nc2ccc(N(C)C)nn2)c(NCCCCCN)n1] | 2-((5-aminopentyl)amino)-N-(6- (dimethylamino)pyridazin-3-yl)-6- methylnicotinamide |
| BL1-16 | 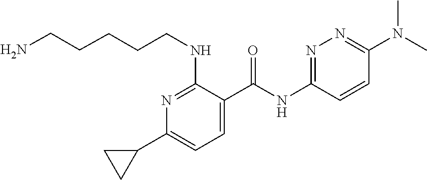 [SMILES: CN(C)c1ccc(NC(=O)c2ccc(C3CC3)nc2NCCCCCN)nn1] | 2-((5-aminopentyl)amino)-6- cyclopropyl-N-(6- (dimethylamino)pyridazin-3- yl)nicotinamide |
| BL1-17 | 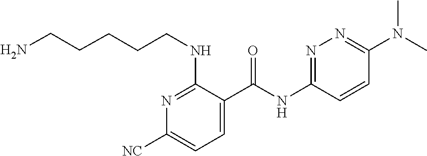 [SMILES: CN(C)c1ccc(NC(=O)c2ccc(C#N)nc2NCCCCCN)nn1] | 2-((5-aminopentyl)amino)-6-cyano- N-(6-(dimethylamino)pyridazin-3- yl)nicotinamide |
| BL1-18 | 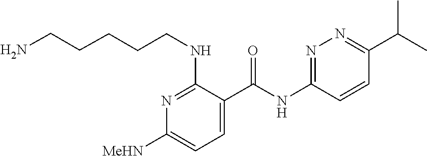 [SMILES: CNc1ccc(C(=O)Nc2ccc(C(C)C)nn2)c(NCCCCCN)n1] | 2-((5-aminopentyl)amino)-N-(6- isopropylpyridazin-3-yl)-6- (methylamino)nicotinamide |
| BL1-19 | 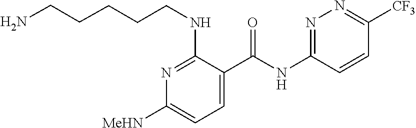 [SMILES: CNc1ccc(C(=O)Nc2ccc(C(F)(F)F)nn2)c(NCCCCCN)n1] | 2-((5-aminopentyl)amino)-6- (methylamino)-N-(6- (trifluoromethyl)pyridazin-3- yl)nicotinamide |
| BL1-20 | 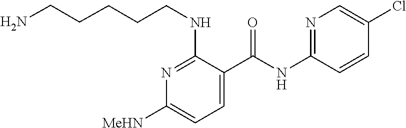 [SMILES: CNc1ccc(C(=O)Nc2ccc(Cl)cn2)c(NCCCCCN)n1] | 2-((5-aminopentyl)amino)-N-(5- chloropyridin-2-yl)-6- (methylamino)nicotinamide |
| BL1-21 | 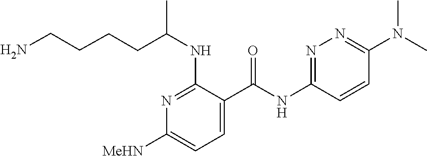 [SMILES: CNc1ccc(C(=O)Nc2ccc(N(C)C)nn2)c(NC(C)CCCCN)n1] | 2-((6-aminohexan-2-yl)amino)-N-(6- (dimethylamino)pyridazin-3-yl)-6- (methylamino)nicotinamide |
| BL1-22 | 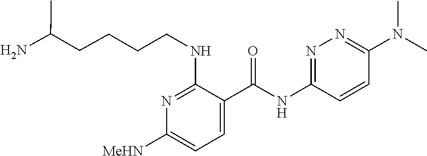 [SMILES: CNc1ccc(C(=O)Nc2ccc(N(C)C)nn2)c(NCCCCC(C)N)n1] | 2-((5-aminohexyl)amino)-N-(6- (dimethylamino)pyridazin-3-yl)-6- (methylamino)nicotinamide |
| BL1-23 | 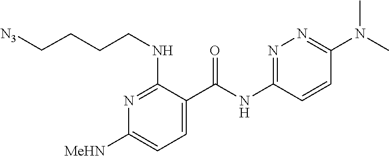 [SMILES: CNc1ccc(C(=O)Nc2ccc(N(C)C)nn2)c(NCCCCN=[N+]=[N-])n1] | 2-((4-azidobutyl)amino)-N-(6- (dimethylamino)pyridazin-3-yl)-6- (methylamino)nicotinamide |
| BL1-24 | 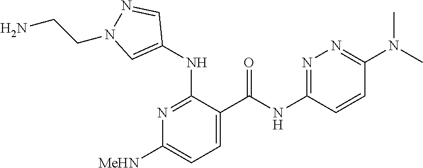 [SMILES: CNc1ccc(C(=O)Nc2ccc(N(C)C)nn2)c(Nc2cnn(CCN)c2)n1] | 2-((1-(2-aminoethyl)-1H-pyrazol-4- yl)amino)-N-(6- (dimethylamino)pyridazin-3-yl)-6- (methylamino)nicotinamide |
| BL1-25 | 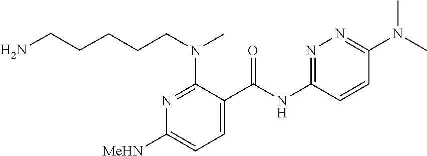 [SMILES: CNc1ccc(C(=O)Nc2ccc(N(C)C)nn2)c(N(C)CCCCCN)n1] | 2-((5-aminopentyl)(methyl)amino)- N-(6-(dimethylamino)pyridazin-3- yl)-6-(methylamino)nicotinamide |
| BL1-26 | 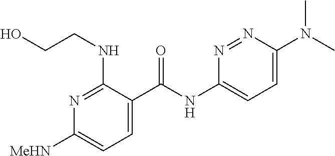 [SMILES: CNc1ccc(C(=O)Nc2ccc(N(C)C)nn2)c(NCCO)n1] | N-(6-(dimethylamino)pyridazin-3- yl)-2-((2-hydroxyethyl)amino)-6- (methylamino)nicotinamide |
| BL1-27 | 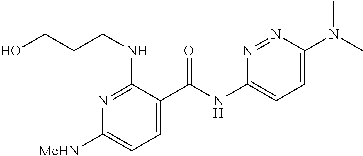 [SMILES: CNc1ccc(C(=O)Nc2ccc(N(C)C)nn2)c(NCCCO)n1] | N-(6-(dimethylamino)pyridazin-3- yl)-2-((3-hydroxypropyl)amino)-6- (methylamino)nicotinamide |
| BL1-28 | 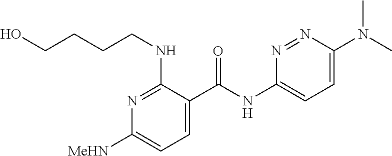 [SMILES: CNc1ccc(C(=O)Nc2ccc(N(C)C)nn2)c(NCCCCO)n1] | N-(6-(dimethylamino)pyridazin-3- yl)-2-((4-hydroxybutyl)amino)-6- (methylamino)nicotinamide |
| BL1-29 | 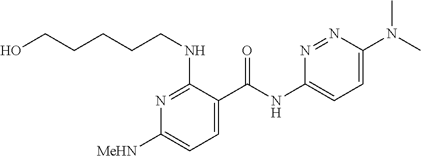 [SMILES: CNc1ccc(C(=O)Nc2ccc(N(C)C)nn2)c(NCCCCCO)n1] | N-(6-(dimethylamino)pyridazin-3- yl)-2-((5-hydroxypentyl)amino)-6- (methylamino)nicotinamide |
| BL1-30 | 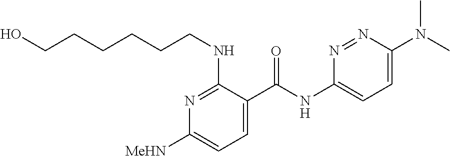 [SMILES: CNc1ccc(C(=O)Nc2ccc(N(C)C)nn2)c(NCCCCCCO)n1] | N-(6-(dimethylamino)pyridazin-3- yl)-2-((6-hydroxyhexyl)amino)-6- (methylamino)nicotinamide |
| BL1-31 | 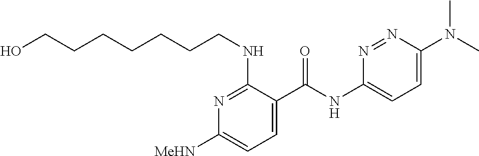 [SMILES: CNc1ccc(C(=O)Nc2ccc(N(C)C)nn2)c(NCCCCCCCO)n1] | N-(6-(dimethylamino)pyridazin-3- yl)-2-((7-hydroxyheptyl)amino)-6- (methylamino)nicotinamide |
| BL1-32 | 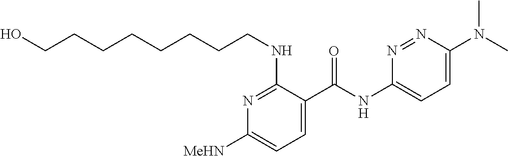 [SMILES: CNc1ccc(C(=O)Nc2ccc(N(C)C)nn2)c(NCCCCCCCCO)n1] | N-(6-(dimethylamino)pyridazin-3- yl)-2-((8-hydroxyoctyl)amino)-6- (methylamino)nicotinamide |
| BL1-33 | 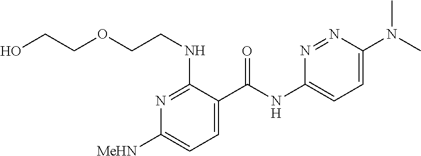 [SMILES: CNc1ccc(C(=O)Nc2ccc(N(C)C)nn2)c(NCCOCCO)n1] | N-(6-(dimethylamino)pyridazin-3- yl)-2-((2-(2- hydroxyethoxy)ethyl)amino)-6- (methylamino)nicotinamide |
| BL1-34 | 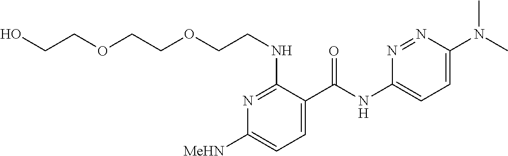 [SMILES: CNc1ccc(C(=O)Nc2ccc(N(C)C)nn2)c(NCCOCCOCCO)n1] | N-(6-(dimethylamino)pyridazin-3- yl)-2-((2-(2-(2- hydroxyethoxy)ethoxy)ethyl)amino)- 6-(methylamino)nicotinamide |
| BL1-35 | 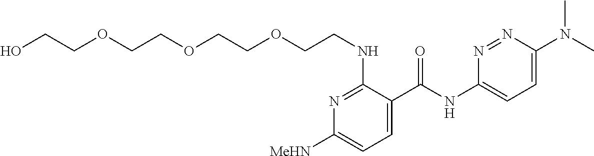 [SMILES: CNc1ccc(C(=O)Nc2ccc(N(C)C)nn2)c(NCCOCCOCCOCCO)n1] | N-(6-(dimethylamino)pyridazin-3- yl)-2-((2-(2-(2-(2- hydroxyethoxy)ethoxy)ethoxy)ethyl) amino)-6- (methylamino)nicotinamide |
| BL1-36 | 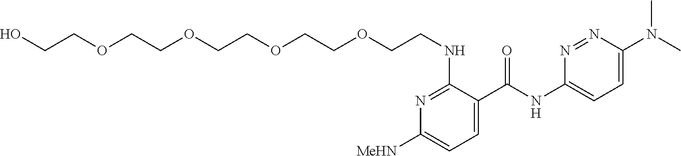 [SMILES: CNc1ccc(C(=O)Nc2ccc(N(C)C)nn2)c(NCCOCCOCCOCCOCCO)n1] | N-(6-(dimethylamino)pyridazin-3- yl)-2-((14-hydroxy-3,6,9,12- tetraoxatetradecyl)amino)-6- (methylamino)nicotinamide |
| BL1-37 | 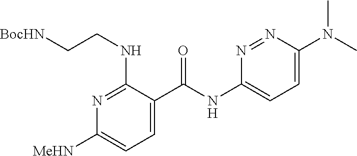 [SMILES: CNc1ccc(C(=O)Nc2ccc(N(C)C)nn2)c(NCCNC(=O)OC(C)(C)C)n1] | tert-butyl (2-((3-((6- (dimethylamino)pyridazin-3- yl)carbamoyl)-6- (methylamino)pyridin-2- yl)amino)ethyl)carbamate |
| BL1-38 | 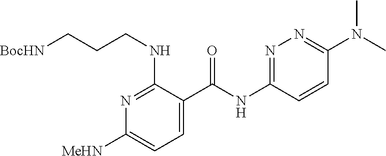 [SMILES: CNc1ccc(C(=O)Nc2ccc(N(C)C)nn2)c(NCCCNC(=O)OC(C)(C)C)n1] | tert-butyl (3-((3-((6- (dimethylamino)pyridazin-3- yl)carbamoyl)-6- (methylamino)pyridin-2- yl)amino)propyl)carbamate |
| BL1-39 | 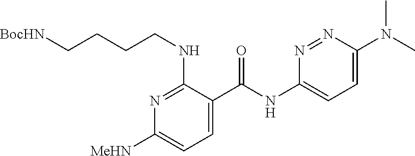 [SMILES: CNc1ccc(C(=O)Nc2ccc(N(C)C)nn2)c(NCCCCNC(=O)OC(C)(C)C)n1] | tert-butyl (4-((3-((6- (dimethylamino)pyridazin-3- yl)carbamoyl)-6- (methylamino)pyridin-2- yl)amino)butyl)carbamate |
| BL1-40 | 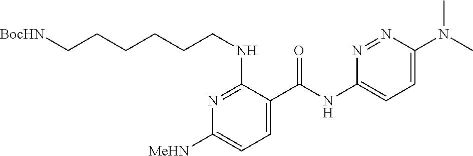 [SMILES: CNc1ccc(C(=O)Nc2ccc(N(C)C)nn2)c(NCCCCCCNC(=O)OC(C)(C)C)n1] | tert-butyl (6-((3-((6- (dimethylamino)pyridazin-3- yl)carbamoyl)-6- (methylamino)pyridin-2- yl)amino)hexyl)carbamate |
| BL1-41 | 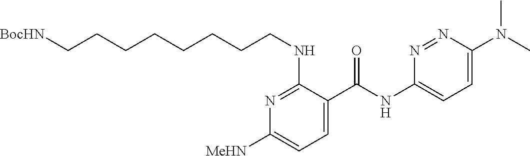 [SMILES: CNc1ccc(C(=O)Nc2ccc(N(C)C)nn2)c(NCCCCCCCCNC(=O)OC(C)(C)C)n1] | 2-((8-aminooctyl)amino)-N-(6- (dimethylamino)pyridazin-3-yl)-6- (methylamino)nicotinamide |
| BL1-42 | 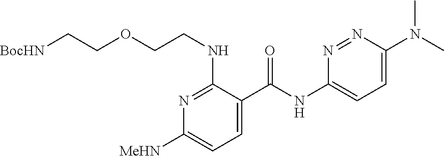 [SMILES: CNc1ccc(C(=O)Nc2ccc(N(C)C)nn2)c(NCCOCCNC(=O)OC(C)(C)C)n1] | tert-butyl (2-(2-((3-((6- (dimethylamino)pyridazin-3- yl)carbamoyl)-6- (methylamino)pyridin-2- yl)amino)ethoxy)ethyl)carbamate |
| BL1-43 | 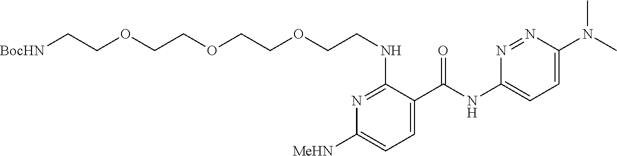 [SMILES: CNc1ccc(C(=O)Nc2ccc(N(C)C)nn2)c(NCCOCCOCCOCCNC(=O)OC(C)(C)C)n1] | tert-butyl (2-(2-(2-((3-((6- (dimethylamino)pyridazin-3- yl)carbamoyl)-6-(methylamino)- pyridin-2-yl)amino)ethoxy)- ethoxy)ethyl)carbamate |
| BL1-44 | 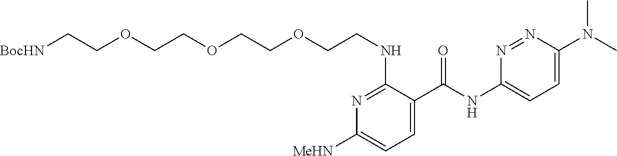 [SMILES: CNc1ccc(C(=O)Nc2ccc(N(C)C)nn2)c(NCCOCCOCCOCCNC(=O)OC(C)(C)C)n1] | tert-butyl (2-(2-(2-(2-((3-((6- (dimethylamino)pyridazin-3- yl)carbamoyl)-6- (methylamino)pyridin-2- yl)amino)ethoxy)ethoxy)ethoxy) ethyl)carbamate |
| BL1-45 | 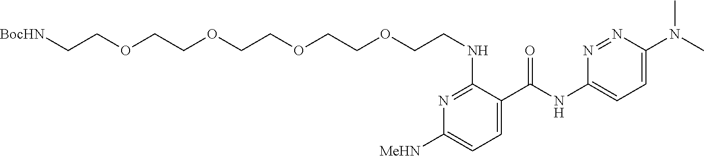 [SMILES: CNc1ccc(C(=O)Nc2ccc(N(C)C)nn2)c(NCCOCCOCCOCCOCCNC(=O)OC(C)(C)C)n1] | tert-butyl (14-((3-((6- (dimethylamino)pyridazin-3- yl)carbamoyl)-6-(methylamino)- pyridin-2-yl)amino)-3,6,9,12- tetraoxatetradecyl)carbamate |
| BL1-46 | 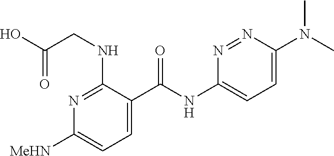 [SMILES: CNc1ccc(C(=O)Nc2ccc(N(C)C)nn2)c(NCC(=O)O)n1] | (3-((6-(dimethylamino)pyridazin-3- yl)carbamoyl)-6- (methylamino)pyridin-2-yl)glycine |
| BL1-47 | 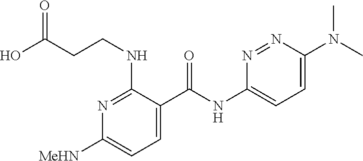 [SMILES: CNc1ccc(C(=O)Nc2ccc(N(C)C)nn2)c(NCCC(=O)O)n1] | 3-((3-((6-(dimethylamino)pyridazin- 3-yl)carbamoyl)-6- (methylamino)pyridin-2- yl)amino)propanoic acid |
| BL1-48 | 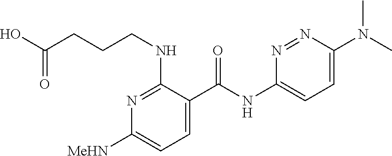 [SMILES: CNc1ccc(C(=O)Nc2ccc(N(C)C)nn2)c(NCCCC(=O)O)n1] | 4-((3-((6-(dimethylamino)pyridazin- 3-yl)carbamoyl)-6- (methylamino)pyridin-2- yl)amino)butanoic acid |
| BL1-49 | 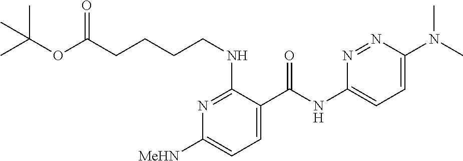 [SMILES: CNc1ccc(C(=O)Nc2ccc(N(C)C)nn2)c(NCCCCC(=O)OC(C)(C)C)n1] | tert-butyl 5-((3-((6- (dimethylamino)pyridazin-3- yl)carbamoyl)-6- (methylamino)pyridin-2- yl)amino)pentanoate |
| BL1-50 | 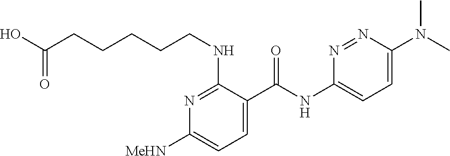 [SMILES: CNc1ccc(C(=O)Nc2ccc(N(C)C)nn2)c(NCCCCCC(=O)O)n1] | 6-((3-((6-(dimethylamino)pyridazin- 3-yl)carbamoyl)-6-(methylamino)- pyridin-2-yl)amino)hexanoic acid |
| BL1-51 | 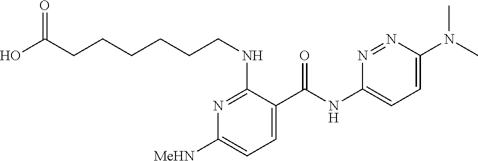 [SMILES: CNc1ccc(C(=O)Nc2ccc(N(C)C)nn2)c(NCCCCCCC(=O)O)n1] | 7-((3-((6- (dimethylamino)109yridazine-3- yl)carbamoyl)-6- (methylamino)109yridazi-2- yl)amino)heptanoic acid |
| BL1-52 | 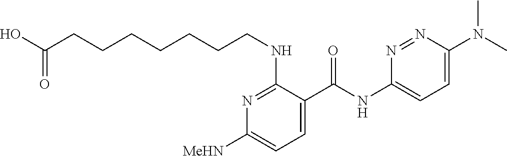 [SMILES: CNc1ccc(C(=O)Nc2ccc(N(C)C)nn2)c(NCCCCCCCC(=O)O)n1] | 8-((3-((6- (dimethylamino)109yridazine-3- yl)carbamoyl)-6- (methylamino)pyridazi-2- yl)amino)octanoic acid |
| BL1-53 | 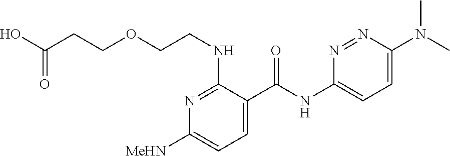 [SMILES: CNc1ccc(C(=O)Nc2ccc(N(C)C)nn2)c(NCCOCCC(=O)O)n1] | 3-(2-((3-((6- (dimethylamino)pyridazine-3- yl)carbamoyl)-6- (methylamino)109yridazi-2- yl)amino)ethoxy)propanoic acid |
| BL1-54 | 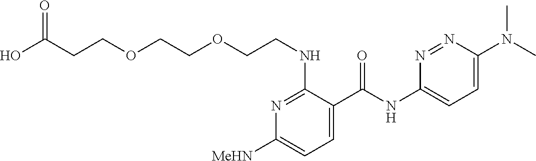 [SMILES: CNc1ccc(C(=O)Nc2ccc(N(C)C)nn2)c(NCCOCCOCCC(=O)O)n1] | 3-(2-(2-((3-((6- (dimethylamino)pyridazine-3- yl)carbamoyl)-6-(methylamino)- pyridin-2-yl)amino)ethoxy)- ethoxy)propanoic acid |
| BL1-55 | 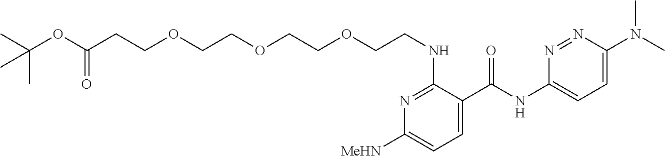 [SMILES: CNc1ccc(C(=O)Nc2ccc(N(C)C)nn2)c(NCCOCCOCCOCCC(=O)OC(C)(C)C)n1] | tert-butyl 3-(2-(2-(2-((3-((6- (dimethylamino)pyridazin-3- yl)carbamoyl)-6-(methylamino)- pyridin-2-yl)amino)ethoxy)- ethoxy)ethoxy)propanoate |
| BL1-56 | 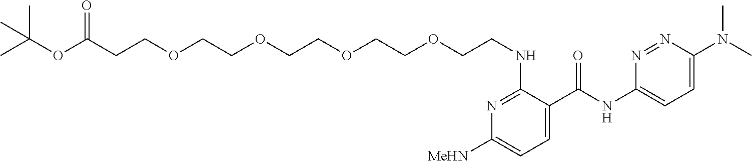 [SMILES: CNc1ccc(C(=O)Nc2ccc(N(C)C)nn2)c(NCCOCCOCCOCCOCCC(=O)OC(C)(C)C)n1] | Tert-butyl 1-((3-((6- (dimethylamino)pyridazin-3- yl)carbamoyl)-6-(methylamino)- pyridin-2-yl)amino)-3,6,9,12- tetraoxapentadecan-15-oate |

### Target Protein Binding Moieties

Disclosed herein, in some embodiments, are compounds comprising a protein binding moiety (PBM) which binds to a target protein. The compound may comprise a heterobifunctional molecule comprising the protein binding moiety.

Disclosed herein, in some embodiments, the target protein is cyclin-dependent kinase 4 (CDK4) and/or cyclin dependent kinase 6 (CDK6). In some embodiments, the target protein is CDK4. In some embodiments, the target protein is CDK6.

In some embodiments, A is a PBM that binds to CDK4 and/or a CDK6. In some such embodiments, A is a PBM that binds to CDK4. In some such embodiments, A is a PBM that binds to CDK6. In some such embodiments, A is a PBM that binds to CDK4 and CDK6.

In some embodiments, A is a PBM having the structure of Formula (A):

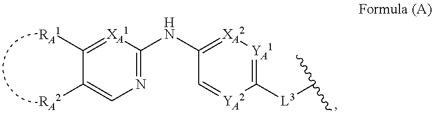

wherein:
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, optionally substituted C₆-C₁₂ aryl, or optionally substituted 5-13 membered heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or 3-8 membered heterocyclyl; or
R_{A}¹ and R_{A}², together with the atom(s) to which they are attached optionally form an optionally substituted C₃-C₈ cycloalkyl, 3-8 membered heterocyclyl, C₆-C₁₂ aryl or 5-13 membered heteroaryl;
L³ is a divalent group selected from —R_{A}^{3A}-R_{A}^{3B}—, wherein R_{A}^{3A} and R_{A}^{3B} are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₂ cycloalkylene, 3-12 membered heterocyclene, C₆-C₁₂ arylene, or 5-13 membered heteroarylene, provided two —O— and/or —S— are not contiguous;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl;
R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl; or
R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl or 5-13 membered heteroaryl; or
R_{A}⁸ and R_{A}⁹ together with the atom(s) to which they are connected optionally form a 3-20 membered heterocyclyl ring.

In some embodiments, R_{A}¹ and R_{A}² together with the atom(s) to which they are connected, form an optionally substituted 3-8 membered heterocyclyl or 5-13 membered heteroaryl.

In some embodiments, A is a PBM of Formula (A) having the structure of Formula (A1), (A2), or (A3):

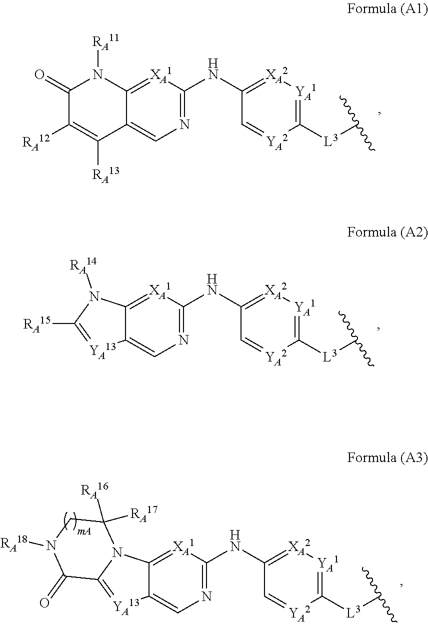

wherein:
Y_{A}³ is CR_{A}¹⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or 3-8 membered heterocyclyl, C₆-C₁₂ aryl, or 5-13 membered heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹, wherein R_{A}²⁰ and R_{A}²¹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or 3-8 membered heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or 3-8 membered heterocyclyl;
R_{A}¹⁶ and R_{A}¹⁷ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, 3-8 membered heterocyclyl, C₆-C₁₂ aryl, or 5-13 membered heteroaryl; or
R_{A}¹⁶ and R_{A}¹⁷, together with the atom(s) to which they are connected optionally form C₃-C₈ cycloalkyl, or 3-8 membered heterocyclyl; and
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or 3-8 membered heterocyclyl; and
m_{A} is 0, 1, or 2.

In some embodiments, the PBM of Formula (A) has the structure of Formula (A1).

In some embodiments, the PBM of Formula (A) has the structure of Formula (A2).

In some embodiments, the PBM of Formula (A) has the structure of Formula (A3).

In some embodiments, m_{A} is 1.

In some embodiments, R_{A}¹ is optionally substituted C₆-C₁₂ aryl, or optionally substituted 3-13 membered heteroaryl.

In some embodiments, the PBM of Formula (A) has the structure of Formula (A4):

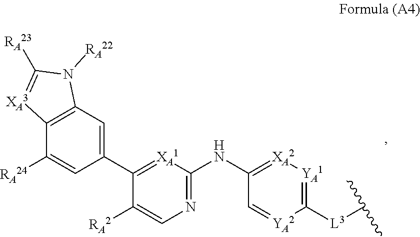

wherein:
X_{A}²³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, C₆-C₁₂ aryl, or 3-13 membered heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.

In some embodiments, X_{A}¹, X_{A}², and X_{A}³ are each N. In some embodiments, X_{A}¹ is N. In some embodiments, X_{A}² is N. In some embodiments, X_{A}³ is N.

In some embodiments, X_{A}¹ is CR_{A}⁴. In some embodiments, X_{A}² is CR_{A}⁴. In some embodiments, X_{A}³ is CR_{A}⁴. In some embodiments, X_{A}¹ is CH. In some embodiments, X_{A}² is CH. In some embodiments, X_{A}³ is CH.

In some embodiments, Y_{A}¹, Y_{A}², and Y_{A}³ are each N. In some embodiments, Y_{A}¹ is N. In some embodiments, Y_{A}² is N. In some embodiments, Y_{A}³ is N

In some embodiments, Y_{A}¹ is CR_{A}⁴. In some embodiments, Y_{A}² is CR_{A}⁴. In some embodiments, Y_{A}³ is CR_{A}⁴. In some embodiments, Y_{A}¹, Y_{A}², and Y_{A}³ are each CH.

In some embodiments, R_{A}², R_{A}⁴, R_{A}¹³, R_{A}¹⁹, R_{A}²³, and R_{A}²⁴ are each independently selected from hydrogen, halogen, C₁-C₃ alkyl, or C₃-C₆ cycloalkyl. In some embodiments, R_{A}², R_{A}⁴, R_{A}¹³, R_{A}¹⁹, R_{A}²³, and R_{A}²⁴ are each independently selected from hydrogen, F, Cl, CH₃, CH₂CH₃, CH(CH₃)₂, CF₃, CHF₂, cyclopropyl, or cyclobutyl.

In some embodiments, R_{A}¹¹ and R_{A}¹⁴ are each independently selected from hydrogen, C₁-C₈ alkyl, C₃-C₈ cycloalkyl, or C₂-C₈ heterocyclyl. In some embodiments, R_{A}¹¹ and R_{A}¹⁴ are each independently selected from C₁-C₈alkyl, or C₃-C₈ cycloalkyl. In some embodiments, R_{A}¹¹ and R_{A}¹⁴ are each independently selected from C₁-C₈ alkyl. In some embodiments, R_{A}¹¹ and R_{A}¹⁴ are each independently selected from C₃-C₅ cycloalkyl.

In some embodiments, R_{A}¹² and R_{A}¹⁵ are each independently selected from R_{A}²⁰, COR_{A}²⁰, or CONR_{A}²⁰R_{A}²¹, wherein R_{A}²⁰ and R_{A}²¹ are each independently selected from C₁-C₈ alkyl, C₃-C₈ cycloalkyl, or C₂-C₈ heterocyclyl. In some embodiments, R_{A}¹² and R_{A}¹⁵ are each independently selected from COR_{A}²⁰, or CONR_{A}²⁰R_{A}²¹, wherein R_{A}²⁰ and R_{A}²¹ are each independently selected from C₁-C₈ alkyl.

In some embodiments, R_{A}¹⁶ and R_{A}¹⁷ are each independently selected from hydrogen, C₁-C₈ alkyl, C₃-C₈ cycloalkyl, or C₂-C₈ heterocyclyl. In some embodiments, R_{A}¹⁶ and R_{A}¹⁷ are each independently selected from C₁-C₈ alkyl. In some embodiments, R_{A}¹⁶ and R_{A}¹⁷ are each independently selected from C₃-C₈ cycloalkyl. In some embodiments, R_{A}¹⁶ and R_{A}¹⁷ are each independently selected from C₂-C₈ heterocyclyl.

In some embodiments, R_{A}¹⁶ and R_{A}¹⁷ together with the atom(s) to which they are connected optionally form a 3-6 membered cycloalkyl or 3-6 membered heterocyclyl ring. In some embodiments, R_{A}¹⁶ and R_{A}¹⁷ together with the atom(s) to which they are connected optionally form a 3-6 membered cycloalkyl. In some embodiments, R_{A}¹⁶ and R_{A}¹⁷ together with the atom(s) to which they are connected optionally form a 3-6 membered heterocyclyl ring. In some embodiments, R_{A}¹⁸ and R_{A}²² are each independently selected from hydrogen, C₁-C₈ alkyl, C₃-C₈ cycloalkyl, or C₂-C₈ heterocyclyl. In some embodiments, R_{A}¹⁸ and R_{A}²² are each independently selected from H, CH₃, CH₂CH₃, CH(CH₃)₂, CF₃, CHF₂, cyclopropyl, or cyclobutyl.

In some embodiments, L³ is a divalent group selected from —R_{A}^{3A}-R_{A}^{3B}—, wherein R_{A}BA and R_{A}^{3B} are each independently a bond, —O—, —S—, —NR_{A}⁷—, —C(═O)—, —C(═O)NR_{A}⁷—, —S(═O)—, —S(═O)NR_{A}⁷-, —S(═O)₂—, —S(═O)₂NR_{A}⁷—, C₁-C₈alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, 3-12 membered heterocyclene, C₆-C₁₂ arylene, or 5-13 membered heteroarylene, provided two —O— and/or —S— are not contiguous. In some such embodiments, R_{A}^{3A} and R_{A}³B are each independently a bond, —O—, —S—, —NR_{A}⁷—, —C(═O)—, —C(═O)NR_{A}⁷—, —S(═O)—, —S(═O)NR_{A}⁷—, —S(═O)₂—, —S(═O)₂NR_{A}⁷-. In some such embodiments, R_{A}^{3A} and R_{A}^{3B} are each independently C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, 3-12 membered heterocyclene, C₆-C₁₂ arylene, or 5-13 membered heteroarylene.

In some embodiments, R_{A}^{3A} is selected from a bond, —O—, —S—, —NR_{A}⁷—, —C(═O)—, —C(═O)NR_{A}⁷-, —S(═O)—, —S(═O)NR_{A}⁷-, —S(═O)₂—, —S(═O)₂NR_{A}⁷-; and R_{A}^{3B} is selected from C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, 3-12 membered heterocyclene, C₆-C₁₂ arylene, or 5-13 membered heteroarylene.

In some embodiments, R_{A}^{3B} is selected from a bond, —O—, —S—, —NR_{A}⁷—, —C(═O)—, —C(═O)NR_{A}⁷-—S(═O)—, —S(═O)NR_{A}⁷—, —S(═O)₂—, —S(═O)₂NR_{A}⁷-; and R_{A}^{3A} is selected from C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₃ cycloalkylene, 3-12 membered heterocyclene, C₆-C₁₂ arylene, or 5-13 membered heteroarylene.

In some embodiments, L³ is a bond, C₁-C₃ alkylene, C₃-C₈ cycloalkylene, C₂-C₈ heteroalkylene, 3-8 membered heterocyclene, —(C₁-C₃ alkylene)-(C₃-C₈ cycloalkylene)-, —(C₁-C₃ alkylene)-(3-8 membered heterocyclene)-, or —(C₁-C₃ alkylene)-(C₂-C₈ heteroalkylene). In some embodiments, L³ is a bond, C₁-C₃ alkylene, C₃-C₁₂ cycloalkylene, C₂-C₈ heteroalkylene, 3-12 membered heterocyclene, —(C₁-C₃ alkylene)-(C₃-C₁₂ cycloalkylene)-, —(C₁-C₃ alkylene)-(3-12 membered heterocyclene)-, or —(C₁-C₃ alkylene)-(C₂-C₈ heteroalkylene).

In some embodiments, L³ is a bond. In some embodiments, L³ is C₁-C₃ alkylene. In some embodiments, L³ is C₃-C₈ cycloalkylene. In some embodiments, L³ is C₂-C₈ heteroalkylene. In some embodiments, L³ is 3-8 membered heterocyclene. In some embodiments, L³ is —(C₁-C₃ alkylene)-(C₃-C₈ cycloalkylene)-. In some embodiments, L³ is —(C₁-C₃ alkylene)-(3-8 membered heterocyclene)-. In some embodiments, L³ is —(C₁-C₃ alkylene)-(C₂-C₈ heteroalkylene). In some embodiments, L³ is C₃-C₁₂ cycloalkylene. In some embodiments, L³ is 3-12 membered heterocyclene. In some embodiments, L³ is —(C₁-C₃ alkylene)-(C₃-C₁₂ cycloalkylene)-. In some embodiments, L³ is —(C₁-C₃ alkylene)-(3-12 membered heterocyclene)-.

In some embodiments, L³ is —R_{A}^{3A}-R_{A}^{3B}—, wherein R_{A}^{3A} is a bond or C₁-C₈ alkylene, and R_{A}³B is a 3-12 membered heterocyclene or C₃-C₁₃ cycloalkylene. In some such embodiments, R_{A}^{3A} is a bond, and R_{A}^{3B} is a 3-12 membered heterocyclene. In some such embodiments, R_{A}^{3A} is a bond, and R_{A}^{3B} is a C₃-C₁₃ cycloalkylene. In some such embodiments, R_{A}^{3A} is a C₁-C₈ alkylene, and R_{A}^{3B} is a 3-12 membered heterocyclene or C₃-C₁₃ cycloalkylene. In some such embodiments, R_{A}^{3A} is a C₁-C₈ alkylene, and R_{A}^{3B} is a 3-12 membered heterocyclene. In some such embodiments, R_{A}^{3A} is a C₁-C₈ alkylene, and R_{A}^{3B} is a C₃-C₁₃ cycloalkylene.

In some embodiments, L is —R_{A}^{3A}-R_{A}^{3B}—, wherein R_{A}^{3A} is a 3-12 membered heterocyclene or C₃-C₁₃ cycloalkylene, and R_{A}^{3B} is a 3-12 membered heterocyclene or C₃-C₁₃ cycloalkylene. In some such embodiments, R_{A}^{3A} is a 3-12 membered heterocyclene, and R_{A}^{3B} is a 3-12 membered heterocyclene. In some such embodiments, R_{A}^{3A} is a 3-12 membered heterocyclene, and R_{A}^{3B} is a C₃-C₁₃ cycloalkylene.

In some embodiments, L³ is —R_{A}^{3A}-R_{A}^{3B}—, wherein R_{A}^{3A} is a 3-12 membered heterocyclene or C₃-C₁₃ cycloalkylene, and R_{A}^{3B} is —NR_{A}⁷-. In some such embodiments, R_{A}^{3A} is a 3-12 membered heterocyclene, and R_{A}³B is —NR_{A}⁷—. In some such embodiments, R_{A}^{3A} is a C₃-C₁₃ cycloalkylene, and R_{A}^{3B} is —NR_{A}⁷—.

In some embodiments L³ is selected from:

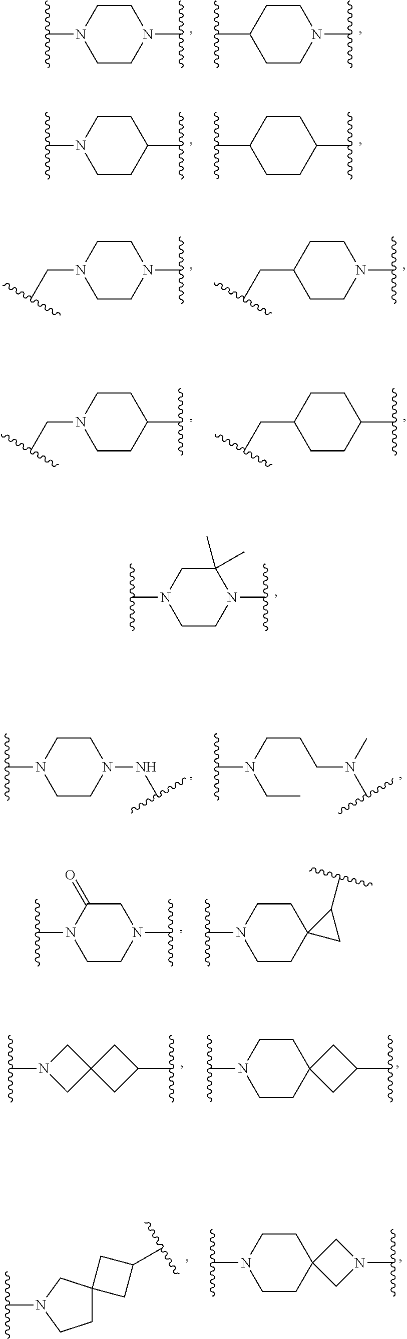

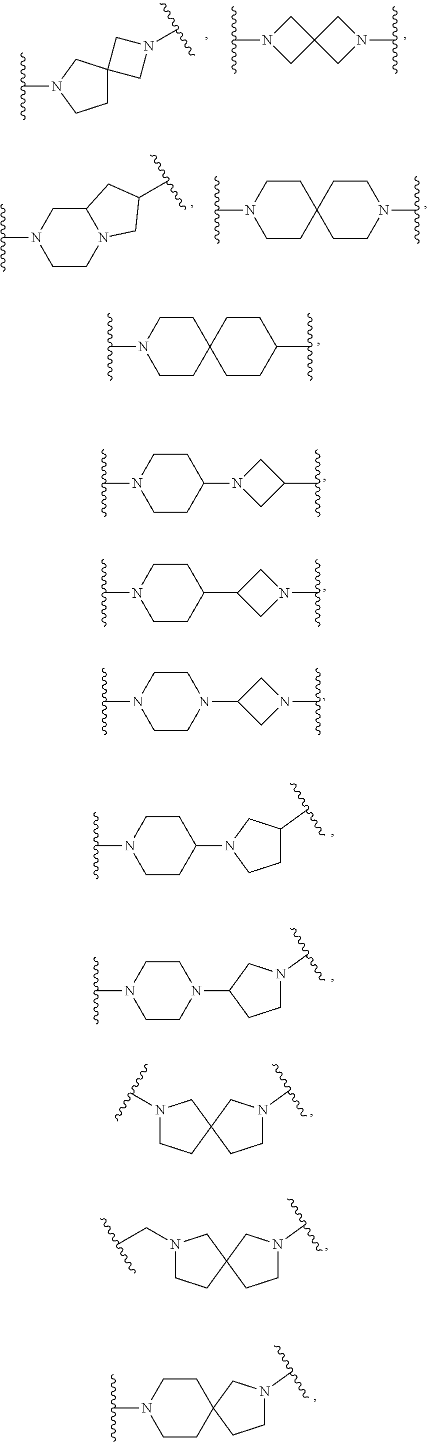

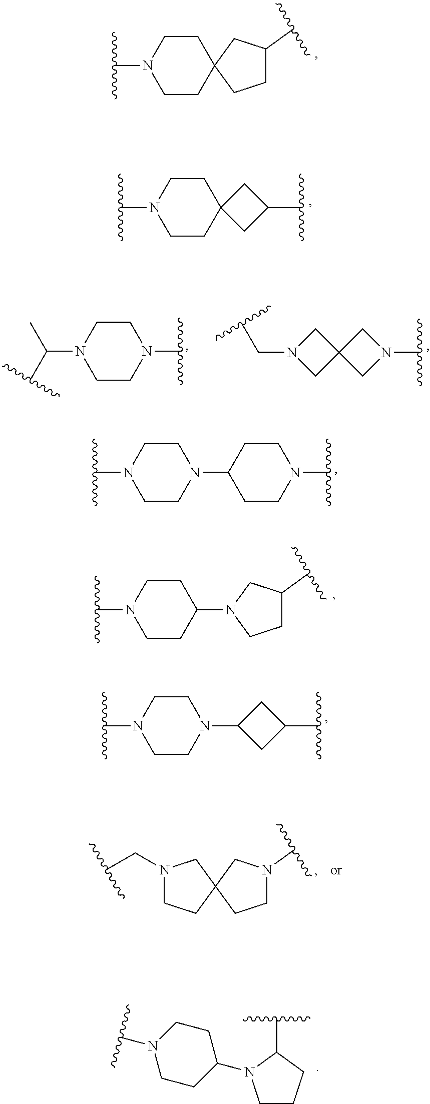

In some embodiments, L³ is a bond,

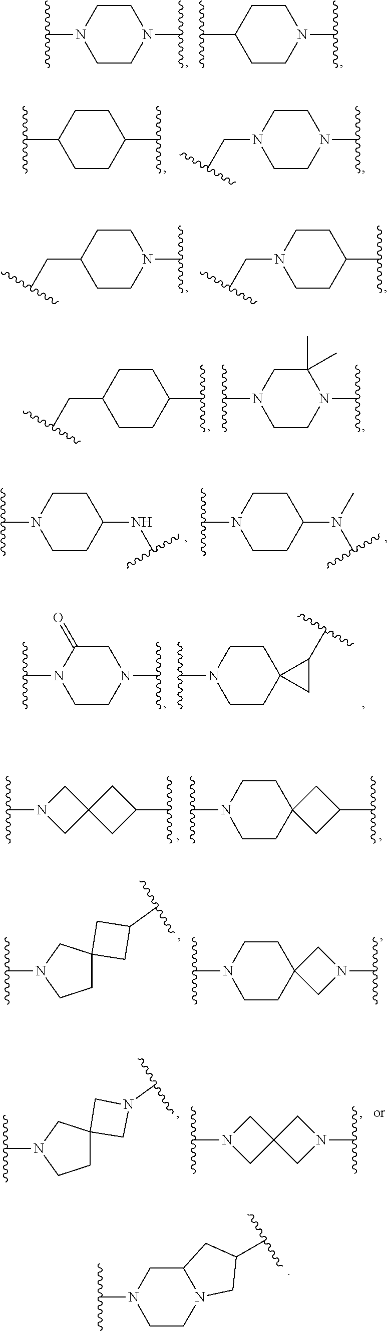

In some embodiments, L³ is

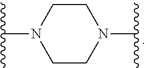 [SMILES: *N1CCN(*)CC1]

In some embodiments, L³ is

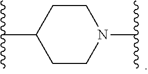 [SMILES: *C1CCN(*)CC1]

In some embodiments, L³ is

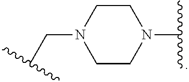 [SMILES: *CN1CCN(*)CC1]

In some embodiments, L³ is

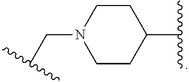 [SMILES: *CN1CCC(*)CC1]

In some embodiments, L³ is

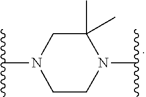 [SMILES: *N1CCN(*)C(C)(C)C1]

In some embodiments, L³ is

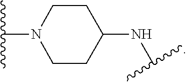 [SMILES: *NC1CCN(*)CC1]

In some preferred embodiments, A is a PBM of Formula (A) having the structure of Formula (A5), (A6), (A7), (A8) or (A9):

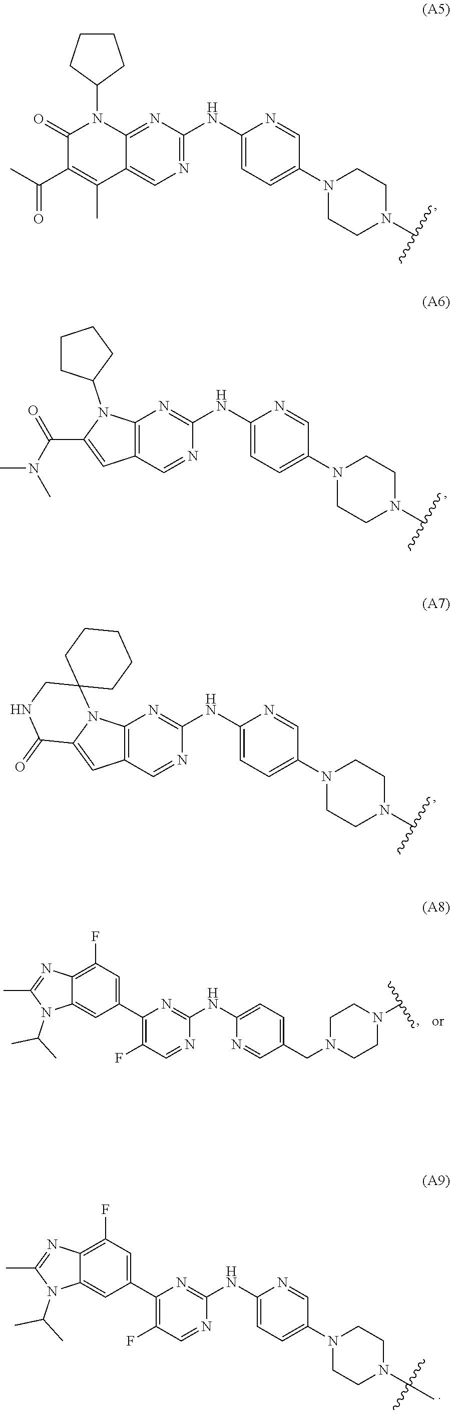 [SMILES: *N1CCN(c2ccc(Nc3ncc4c(C)c(C(C)=O)c(=O)n(C5CCCC5)c4n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc(F)c(-c4cc(F)c5nc(C)n(C(C)C)c5c4)n3)nc2)CC1]

In particularly preferred embodiments, A is a PBM having the structure of Formula (A5).

In some embodiments, A is a PBM having the structure of Formula (A6).

In some embodiments, A is a PBM having the structure of Formula (A7).

In some embodiments, A is a PBM having the structure of Formula (A8).

In some embodiments, A is a PBM having the structure of Formula (A9).

In some embodiments, the protein of interest (POI) is CCND. In some embodiments, the protein of interest is CCND1. In some embodiments, the protein of interest is CCND2. In some embodiments, the protein of interest is CCND3.

Exemplary protein binding moieties are shown in Table 2.

TABLE 2

| Cpd. | | |
|---|---|---|
| No. | Structure | Chemical Name |
| B2-1 | 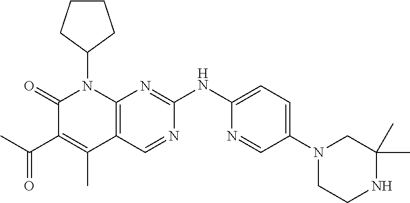 [SMILES: CC(=O)c1c(C)c2cnc(Nc3ccc(N4CCNC(C)(C)C4)cn3)nc2n(C2CCCC2)c1=O] | 6-acetyl-8-cyclopentyl-2-((5-(3,3- dimethylpiperazin-1-yl)pyridin-2- yl)amino)-5-methylpyrido[2,3- d]pyrimidin-7(8H)-one |
| B2-2 | 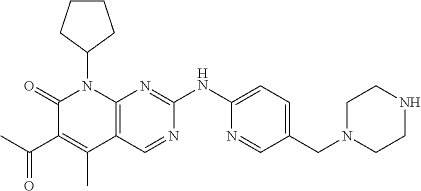 [SMILES: CC(=O)c1c(C)c2cnc(Nc3ccc(CN4CCNCC4)cn3)nc2n(C2CCCC2)c1=O] | 6-acetyl-8-cyclopentyl-5-methyl-2- ((5-(piperazin-1-ylmethyl)pyridin-2- yl)amino)pyrido[2,3-d]pyrimidin- 7(8H)-one |
| B2-3 | 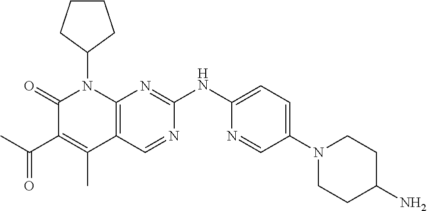 [SMILES: CC(=O)c1c(C)c2cnc(Nc3ccc(N4CCC(N)CC4)cn3)nc2n(C2CCCC2)c1=O] | 6-acetyl-2-((5-(4-aminopiperidin-1- yl)pyridin-2-yl)amino)-8- cyclopentyl-5-methylpyrido[2,3- d]pyrimidin-7(8H)-one |
| B2-4 | 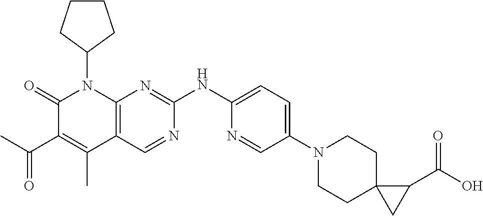 [SMILES: CC(=O)c1c(C)c2cnc(Nc3ccc(N4CCC5(CC4)CC5C(=O)O)cn3)nc2n(C2CCCC2)c1=O] | 6-(6-((6-acetyl-8-cyclopentyl-5- methyl-7-oxo-7,8-dihydropyrido[2,3- d]pyrimidin-2-yl)amino)pyridin-3- yl)-6-azaspiro[2.5]octane-1- carboxylic acid |
| B2-5 | 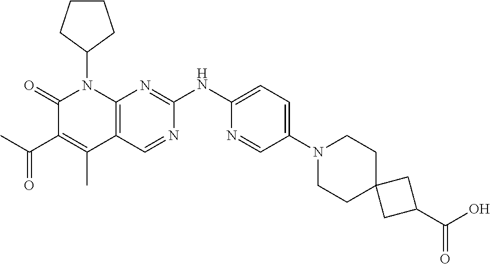 [SMILES: CC(=O)c1c(C)c2cnc(Nc3ccc(N4CCC5(CC4)CC(C(=O)O)C5)cn3)nc2n(C2CCCC2)c1=O] | 7-(6-((6-acetyl-8-cyclopentyl-5- methyl-7-oxo-7,8-dihydropyrido[2,3- d]pyrimidin-2-yl)amino)pyridin-3- yl)-7-azaspiro[3.5]nonane-2- carboxylic acid |
| B2-6 | 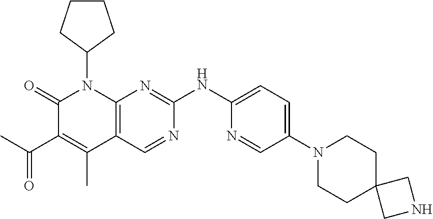 [SMILES: CC(=O)c1c(C)c2cnc(Nc3ccc(N4CCC5(CC4)CNC5)cn3)nc2n(C2CCCC2)c1=O] | 2-((5-(2,7-diazaspiro[3.5]nonan-7- yl)pyridin-2-yl)amino)-6-acetyl-8- cyclopentyl-5-methylpyrido[2,3- d]pyrimidin-7(8H)-one |
| B2-7 | 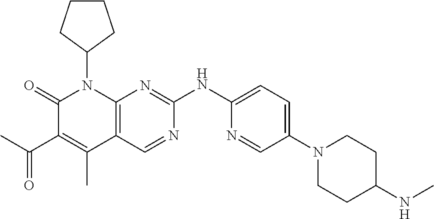 [SMILES: CNC1CCN(c2ccc(Nc3ncc4c(C)c(C(C)=O)c(=O)n(C5CCCC5)c4n3)nc2)CC1] | 2-((5-(3,9-diazaspiro[5.5]undecan-3- yl)pyridin-2-yl)amino)-6-acetyl-8- cyclopentyl-5-methylpyrido[2,3- d]pyrimidin-7(8H)-one |
| B2-8 | 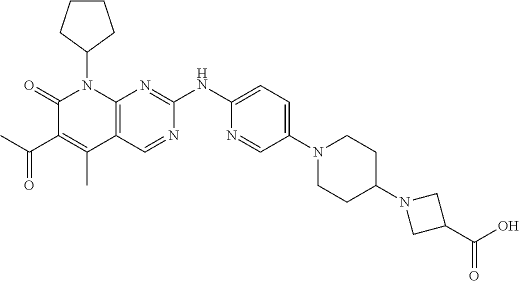 [SMILES: CC(=O)c1c(C)c2cnc(Nc3ccc(N4CCC(N5CC(C(=O)O)C5)CC4)cn3)nc2n(C2CCCC2)c1=O] | 6-acetyl-8-cyclopentyl-5-methyl-2- ((5-(4-(methylamino)piperidin-1- yl)pyridin-2-yl)amino)pyrido[2,3- d]pyrimidin-7(8H)-one |
| B2-9 | 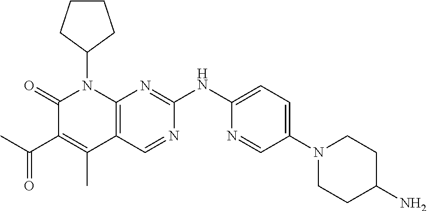 [SMILES: CC(=O)c1c(C)c2cnc(Nc3ccc(N4CCC(N)CC4)cn3)nc2n(C2CCCC2)c1=O] | 1-(1-(6-((6-acetyl-8-cyclopentyl-5- methyl-7-oxo-7,8-dihydropyrido[2,3- d]pyrimidin-2-yl)amino)pyridin-3- yl)piperidin-4-yl)azetidine-3- carboxylic acid |
| B2- 10 | 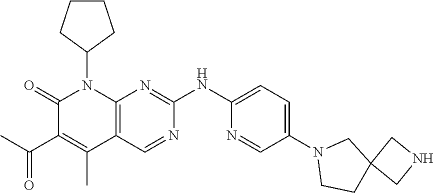 [SMILES: CC(=O)c1c(C)c2cnc(Nc3ccc(N4CCC5(CNC5)C4)cn3)nc2n(C2CCCC2)c1=O] | 2-((5-(2,6-diazaspiro[3.4]octan-6- yl)pyridin-2-yl)amino)-6-acetyl-8- cyclopentyl-5-methylpyrido[2,3- d]pyrimidin-7(8H)-one |
| B2- 11 | 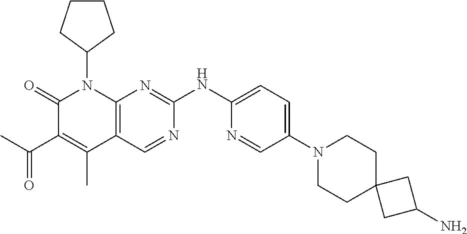 [SMILES: CC(=O)c1c(C)c2cnc(Nc3ccc(N4CCC5(CC4)CC(N)C5)cn3)nc2n(C2CCCC2)c1=O] | 6-acetyl-2-((5-(2-amino-7- azaspiro[3.5]nonan-7-yl)pyridin-2- yl)amino)-8-cyclopentyl-5- methylpyrido[2,3-d]pyrimidin- 7(8H)-one |
| B2- 1H2 | 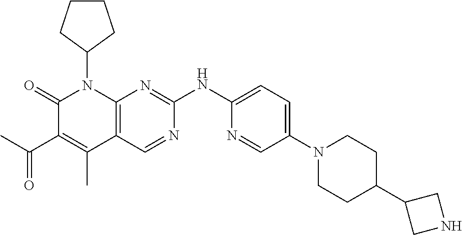 [SMILES: CC(=O)c1c(C)c2cnc(Nc3ccc(N4CCC(C5CNC5)CC4)cn3)nc2n(C2CCCC2)c1=O] | 6-acetyl-2-((5-(4-(azetidin-3- yl)piperidin-1-yl)pyridin-2- yl)amino)-8-cyclopentyl-5- methylpyrido[2,3-d]pyrimidin- 7(8H)-one |
| B2- 13 | 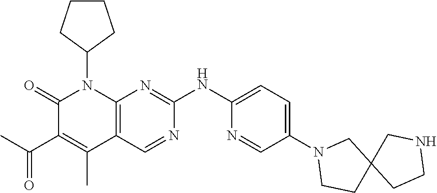 [SMILES: CC(=O)c1c(C)c2cnc(Nc3ccc(N4CCC5(CCNC5)C4)cn3)nc2n(C2CCCC2)c1=O] | 2-((5-(2,7-diazaspiro[4.4]nonan-2- yl)pyridin-2-yl)amino)-6-acetyl-8- cyclopentyl-5-methylpyrido[2,3- d]pyrimidin-7(8H)-one |
| B2- 14 | 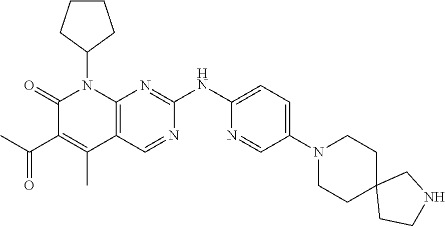 [SMILES: CC(=O)c1c(C)c2cnc(Nc3ccc(N4CCC5(CCNC5)CC4)cn3)nc2n(C2CCCC2)c1=O] | 2-((5-(2,8-diazaspiro[4.5]decan-8- yl)pyridin-2-yl)amino)-6-acetyl-8- cyclopentyl-5-methylpyrido[2,3- d]pyrimidin-7(8H)-one |
| B2- 15 | 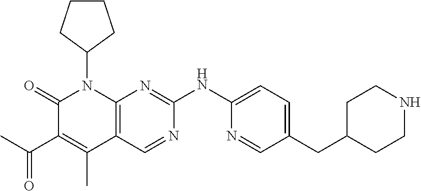 [SMILES: CC(=O)c1c(C)c2cnc(Nc3ccc(CC4CCNCC4)cn3)nc2n(C2CCCC2)c1=O] | 6-acetyl-8-cyclopentyl-5-methyl-2- ((5-(piperidin-4-ylmethyl)pyridin-2- yl)amino)pyrido[2,3-d]pyrimidin- 7(8H)-one |
| B2- 16 | 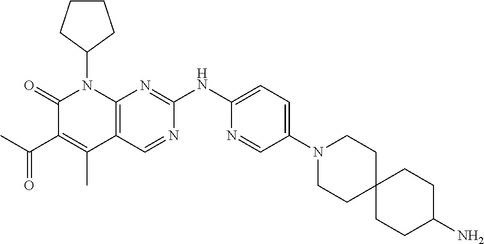 [SMILES: CC(=O)c1c(C)c2cnc(Nc3ccc(N4CCC5(CCC(N)CC5)CC4)cn3)nc2n(C2CCCC2)c1=O] | 6-acetyl-2-((5-(9-amino-3- azaspiro[5.5]undecan-3-yl)pyridin-2- yl)amino)-8-cyclopentyl-5- methylpyrido[2,3-d]pyrimidin- 7(8H)-one |
| B2- 17 | 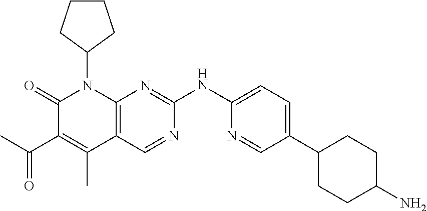 [SMILES: CC(=O)c1c(C)c2cnc(Nc3ccc(C4CCC(N)CC4)cn3)nc2n(C2CCCC2)c1=O] | 6-acetyl-2-((5-(4- aminocyclohexyl)pyridin-2- yl)amino)-8-cyclopentyl-5- methylpyrido[2,3-d]pyrimidin- 7(8H)-one |
| B2- 18 | 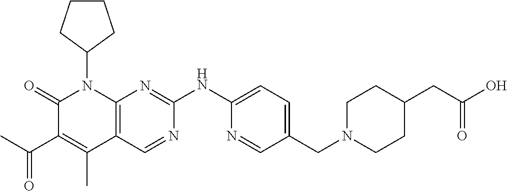 [SMILES: CC(=O)c1c(C)c2cnc(Nc3ccc(CN4CCC(CC(=O)O)CC4)cn3)nc2n(C2CCCC2)c1=O] | 2-(1-((6-((6-acetyl-8-cyclopentyl-5- methyl-7-oxo-7,8-dihydropyrido[2,3- d]pyrimidin-2-yl)amino)pyridin-3- yl)methyl)piperidin-4-yl)acetic acid |
| B2- 19 | 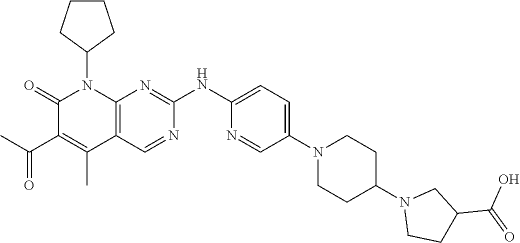 [SMILES: CC(=O)c1c(C)c2cnc(Nc3ccc(N4CCC(N5CCC(C(=O)O)C5)CC4)cn3)nc2n(C2CCCC2)c1=O] | 1-(1-(6-((6-acetyl-8-cyclopentyl-5- methyl-7-oxo-7,8-dihydropyrido[2,3- d]pyrimidin-2-yl)amino)pyridin-3- yl)piperidin-4-yl)pyrrolidine-3- carboxylic acid |
| B2- 20 | 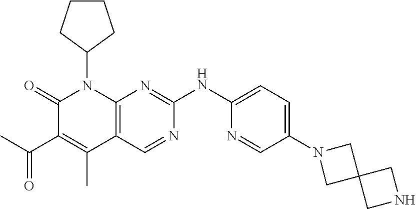 [SMILES: CC(=O)c1c(C)c2cnc(Nc3ccc(N4CC5(CNC5)C4)cn3)nc2n(C2CCCC2)c1=O] | 2-((5-(2,6-diazaspiro[3.3]heptan-2- yl)pyridin-2-yl)amino)-6-acetyl-8- cyclopentyl-5-methylpyrido[2,3- d]pyrimidin-7(8H)-one |
| B2- 21 | 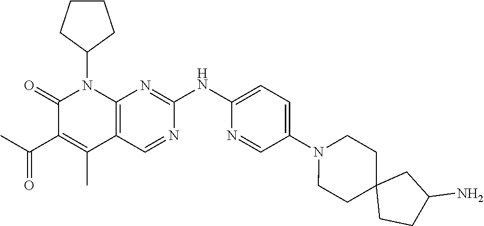 [SMILES: CC(=O)c1c(C)c2cnc(Nc3ccc(N4CCC5(CCC(N)C5)CC4)cn3)nc2n(C2CCCC2)c1=O] | 6-acetyl-2-((5-(2-amino-8- azaspiro[4.5]decan-8-yl)pyridin-2- yl)amino)-8-cyclopentyl-5- methylpyrido[2,3-d]pyrimidin- 7(8H)-one |
| B2- 22 | 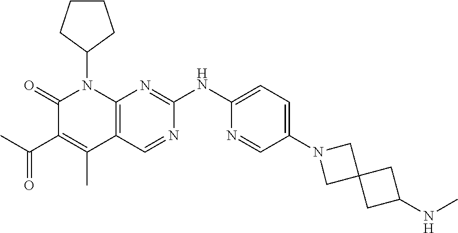 [SMILES: CNC1CC2(C1)CN(c1ccc(Nc3ncc4c(C)c(C(C)=O)c(=O)n(C5CCCC5)c4n3)nc1)C2] | 6-acetyl-8-cyclopentyl-5-methyl-2- ((5-(6-(methylamino)-2- azaspiro[3.3]heptan-2-yl)pyridin-2- yl)amino)pyrido[2,3-d]pyrimidin- 7(8H)-one |
| B2- 23 | 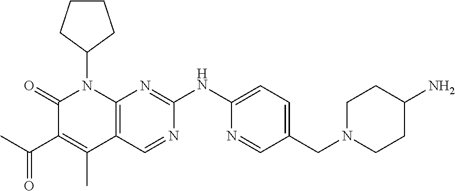 [SMILES: CC(=O)c1c(C)c2cnc(Nc3ccc(CN4CCC(N)CC4)cn3)nc2n(C2CCCC2)c1=O] | 6-acetyl-2-((5-((4-aminopiperidin-1- yl)methyl)pyridin-2-yl)amino)-8- cyclopentyl-5-methylpyrido[2,3- d]pyrimidin-7(8H)-one |
| B2- 24 | 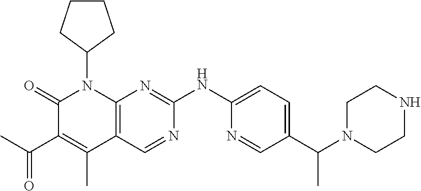 [SMILES: CC(=O)c1c(C)c2cnc(Nc3ccc(C(C)N4CCNCC4)cn3)nc2n(C2CCCC2)c1=O] | 6-acetyl-8-cyclopentyl-5-methyl-2- ((5-(1-(piperazin-1-yl)ethyl)pyridin- 2-yl)amino)pyrido[2,3-d]pyrimidin- 7(8H)-one |
| B2- 25 | 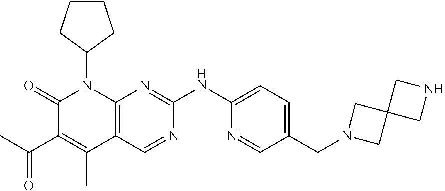 [SMILES: CC(=O)c1c(C)c2cnc(Nc3ccc(CN4CC5(CNC5)C4)cn3)nc2n(C2CCCC2)c1=O] | 2-((5-((2,6-diazaspiro[3.3]heptan-2- yl)methyl)pyridin-2-yl)amino)-6- acetyl-8-cyclopentyl-5- methylpyrido[2,3-d]pyrimidin- 7(8H)-one |
| B2- 26 | 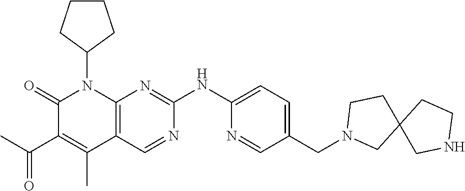 [SMILES: CC(=O)c1c(C)c2cnc(Nc3ccc(CN4CCC5(CCNC5)C4)cn3)nc2n(C2CCCC2)c1=O] | 2-((5-((2,7-diazaspiro[4.4]nonan-2- yl)methyl)pyridin-2-yl)amino)-6- acetyl-8-cyclopentyl-5- methylpyrido[2,3-d]pyrimidin- 7(8H)-one |
| B2- 27 | 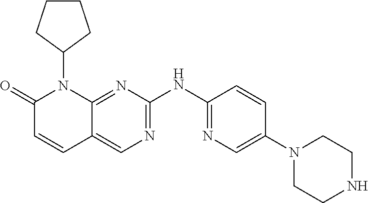 [SMILES: O=c1ccc2cnc(Nc3ccc(N4CCNCC4)cn3)nc2n1C1CCCC1] | 8-cyclopentyl-2-((5-(piperazin-1- yl)pyridin-2-yl)amino)pyrido[2,3- d]pyrimidin-7(8H)-one |
| B2- 28 | 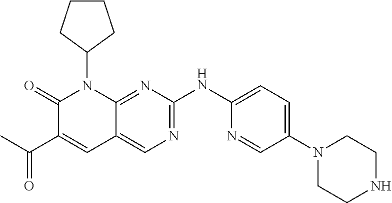 [SMILES: CC(=O)c1cc2cnc(Nc3ccc(N4CCNCC4)cn3)nc2n(C2CCCC2)c1=O] | 6-acetyl-8-cyclopentyl-2-((5- (piperazin-1-yl)pyridin-2- yl)amino)pyrido[2,3-d]pyrimidin- 7(8H)-one |
| B2- 29 | 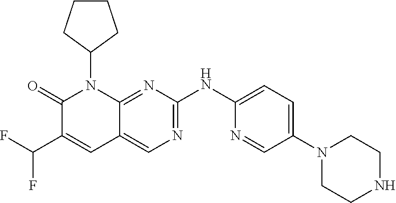 [SMILES: O=c1c(C(F)F)cc2cnc(Nc3ccc(N4CCNCC4)cn3)nc2n1C1CCCC1] | 8-cyclopentyl-6-(difluoromethyl)-2- ((5-(piperazin-1-yl)pyridin-2- yl)amino)pyrido[2,3-d]pyrimidin- 7(8H)-one |

### Heterobifunctonal Compounds

In some embodiments, the compound comprises a heterobifunctional compound of any of Formulae (I)-(XII). Exemplary heterobifunctional compounds of Formulae (I)-(XII) are shown in Table 4A and Table 4B.

TABLE 4A
Representative heterobifunctional compounds.

| Cpd. | |
|---|---|
| No. | Chemical Name |
| CPD-1 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)propyl)amino)-N- |
| | (4,5-dimethylthiazol-2-yl)-6-methylnicotinamide |
| CPD-2 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | methoxypyridazin-3-yl)-6-methylnicotinamide |
| CPD-3 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N- |
| | (4,5-dimethylthiazol-2-yl)-6-methylnicotinamide |
| CPD-4 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N- |
| | (4,5-dimethylthiazol-2-yl)-6-(methylamino)nicotinamide |
| CPD-5 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-(methylamino)nicotinamide |
| CPD-6 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | cyclopropyl-5-methylpyridin-2-yl)-6-(methylamino)nicotinamide |
| CPD-7 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropyl-5-methylpyridin-2-yl)-6-(methylamino)nicotinamide |
| CPD-8 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | (dimethylamino)pyridazin-3-yl)-6-methylnicotinamide |
| CPD-9 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | cyclopropyl-5-methylpyridin-2-yl)-6-methylnicotinamide |
| CPD-10 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | (dimethylamino)pyridazin-3-yl)-6-(methylamino)nicotinamide |
| CPD-11 | 2-((7-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)heptyl)amino)-N-(6- |
| | methoxypyridazin-3-yl)-6-(methylamino)nicotinamide |
| CPD-12 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-3- |
| | fluorobenzamide |
| CPD-13 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-3- |
| | (trifluoromethyl)benzamide |
| CPD-14 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanamido)pentyl)amino)-N- |
| | (6-(dimethylamino)pyridazin-3-yl)-6-(methylamino)nicotinamide |
| CPD-15 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | isopropylpyridazin-3-yl)-6-(methylamino)nicotinamide |
| CPD-16 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-methylnicotinamide |
| CPD-17 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | (methylamino)-N-(6-(pyrrolidin-1-yl)pyridazin-3-yl)nicotinamide |
| CPD-18 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-(isopropylamino)nicotinamide |
| CPD-19 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | (methylamino)-N-(6-(trifluoromethyl)pyridazin-3-yl)nicotinamide |
| CPD-20 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(5- |
| | chloropyridin-2-yl)-6-(methylamino)nicotinamide |
| CPD-21 | 2-((5-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)ethyl)amino)pentyl)amino)-N- |
| | (6-(dimethylamino)pyridazin-3-yl)-6-(methylamino)nicotinamide |
| CPD-22 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)hexyl)amino)-N-(6- |
| | (dimethylamino)pyridazin-3-yl)-6-(methylamino)nicotinamide |
| CPD-23 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)pentyl)(methyl)amino)-N-(6-(dimethylamino)pyridazin-3-yl)-6- |
| | (methylamino)nicotinamide |
| CPD-24 | 2-((1-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)ethyl)- 1H-pyrazol- |
| | 4-yl)amino)-N-(6-(dimethylamino)pyridazin-3-yl)-6-(methylamino)nicotinamide |
| CPD-25 | 2-((2-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)ethyl)-2H-1,2,3- |
| | triazol-4-yl)amino)-N-(6-(dimethylamino)pyridazin-3-yl)-6- |
| | (methylamino)nicotinamide |
| CPD-26 | 2-((3-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)ethyl)isoxazol-5- |
| | yl)amino)-N-(6-(dimethylamino)pyridazin-3-yl)-6-(methylamino)nicotinamide |
| CPD-27 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | cyclopropyl-N-(6-(dimethylamino)pyridazin-3-yl)nicotinamide |
| CPD-28 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | cyano-N-(6-(dimethylamino)pyridazin-3-yl)nicotinamide |
| CPD-29 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)hexan-2-yl)amino)- |
| | N-(6-(dimethylamino)pyridazin-3-yl)-6-(methylamino)nicotinamide |
| CPD-30 | 2-((4-(4-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)-1H-1,2,3-triazol-1- |
| | yl)butyl)amino)-N-(6-(dimethylamino)pyridazin-3-yl)-6- |
| | (methylamino)nicotinamide |
| CPD-31 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropylpyridazin-3-yl)-6-(methylamino)nicotinamide |
| CPD-32 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(1- |
| | methyl-1H-pyrazolo[4,3-b]pyridin-5-yl)-6-(methylamino)nicotinamide |
| CPD-33 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(1- |
| | methyl-1H-pyrrolo[3,2-b]pyridin-5-yl)-6-(methylamino)nicotinamide |
| CPD-34 | 2-((3-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)ethyl)cyclobutyl)amino)-N-(6-(dimethylamino)pyridazin-3-yl)-6- |
| | (methylamino)nicotinamide |
| CPD-35 | 4-(6-((6-acetyl-8-cyclopentyl-5-methyl-7-oxo-7,8-dihydropyrido[2,3-d]pyrimidin- |
| | 2-yl)amino)pyridin-3-yl)-N-(2-(3-((3-((6-(dimethylamino)pyridazin-3- |
| | yl)carbamoyl)-6-(methylamino)pyridin-2-yl)amino)cyclobutyl)ethyl)piperazine-1- |
| | carboxamide |
| CPD-36 | 2-((5-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)ethyl)amino)-5- |
| | oxopentyl)amino)-N-(6-(dimethylamino)pyridazin-3-yl)-6- |
| | (methylamino)nicotinamide |
| CPD-37 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanamido)butyl)amino)-N- |
| | (6-(dimethylamino)pyridazin-3-yl)-6-(methylamino)nicotinamide |
| CPD-38 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(3- |
| | methyl-3H-imidazo[4,5-b]pyridin-5-yl)-6-(methylamino)nicotinamide |
| CPD-39 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-(dimethylamino)pyridazin-3-yl)-6-(methylamino)nicotinamide |
| CPD-40 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N- |
| | (6,7-dihydro-5H-cyclopenta[b]pyridin-2-yl)-6-(methylamino)nicotinamide |
| CPD-41 | 2-((2-(3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclobutyl)ethyl)amino)-N-(6-(dimethylamino)pyridazin-3-yl)-6- |
| | (methylamino)nicotinamide |
| CPD-42 | 2-(4-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)ethyl)piperidin-1- |
| | yl)-N-(6-(dimethylamino)pyridazin-3-yl)-6-(methylamino)nicotinamide |
| CPD-43 | 2-((trans-4-((2-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)acetamido)methyl)cyclohexyl)amino)-N-(6-(dimethylamino)pyridazin-3-yl)-6- |
| | (methylamino)nicotinamide |
| CPD-44 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | cyclopropyl-N-(6-cyclopropyl-5-methylpyridin-2-yl)nicotinamide |
| CPD-45 | 2-((3-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)ethyl)cyclobutyl)amino)-6-cyclopropyl-N-(6-cyclopropyl-5- |
| | methylpyridin-2-yl)nicotinamide |
| CPD-46 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(4- |
| | (dimethylamino)phenyl)-6-(methylamino)nicotinamide |
| CPD-47 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(5- |
| | (dimethylamino)pyridin-2-yl)-6-(methylamino)nicotinamide |
| CPD-48 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(4- |
| | (dimethylamino)cyclohexyl)-6-(methylamino)nicotinamide |
| CPD-49 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(1- |
| | methyl-1H-pyrazol-3-yl)-6-(methylamino)nicotinamide |
| CPD-50 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N- |
| | (1,5-dimethyl-1H-pyrazol-3-yl)-6-(methylamino)nicotinamide |
| CPD-51 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropylpyridin-2-yl)-6-(methylamino)nicotinamide |
| CPD-52 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropylpyridin-2-yl)-6-(methylamino)nicotinamide |
| CPD-53 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropyl-4-methylpyridin-2-yl)-6-(methylamino)nicotinamide |
| CPD-54 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-ethylnicotinamide |
| CPD-55 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(3- |
| | (dimethylamino)cyclopentyl)-6-(methylamino)nicotinamide |
| CPD-56 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(4- |
| | cyclopropyl-5-methylthiazol-2-yl)-6-(methylamino)nicotinamide |
| CPD-57 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-cyclopropyl-5-methylpyridin-2-yl)-6-(methylamino)nicotinamide |
| CPD-58 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridin-3-yl)-6-(methylamino)nicotinamide |
| CPD-59 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | (cyclobutylamino)-N-(6-(dimethylamino)pyridazin-3-yl)nicotinamide |
| CPD-60 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)hexyl)amino)-6- |
| | cyclopropyl-N-(6-cyclopropyl-5-methylpyridin-2-yl)nicotinamide |
| CPD-61 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | cyclopropyl-3-methylpyridin-2-yl)-6-(methylamino)nicotinamide |
| CPD-62 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)-2- |
| | azaspiro[3.3]heptan-6-yl)amino)-N-(6-(dimethylamino)pyridazin-3-yl)-6- |
| | (methylamino)nicotinamide |
| CPD-63 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-isopropylnicotinamide |
| CPD-64 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(3- |
| | (dimethylamino)cyclobutyl)-6-(methylamino)nicotinamide |
| CPD-65 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(3- |
| | ((dimethylamino)methyl)cyclobutyl)-6-(methylamino)nicotinamide |
| CPD-66 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-((methyl-d₃)amino)nicotinamide |
| CPD-67 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)spiro[3.3]heptan-2-yl)-6-(methylamino)nicotinamide |
| CPD-68 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-6-(trifluoromethyl)nicotinamide |
| CPD-69 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6,7-dihydro-5H-cyclopenta[b]pyridin-2-yl)-6- |
| | (methylamino)nicotinamide |
| CPD-70 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(1,5-dimethyl-1H-pyrazol-3-yl)-6-(methylamino)nicotinamide |
| CPD-71 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-N-(6- |
| | (dimethylamino)pyridazin-3-yl)-N-methyl-6-(methylamino)nicotinamide |
| CPD-72 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-(methylamino)nicotinamide |
| CPD-73 | 2-((trans-4-(2-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)acetamido)cyclohexyl)methyl)amino)-N-(6-(dimethylamino)pyridazin-3-yl)-6- |
| | (methylamino)nicotinamide |
| CPD-74 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-cyclopropylpyridin-3-yl)-6-(methylamino)nicotinamide |
| CPD-75 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-6-(methylamino)-N-(6-methylpyridin-2-yl)nicotinamide |
| CPD-76 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-isopropylpyridin-2-yl)-6-(methylamino)nicotinamide |
| CPD-77 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | (methylamino)-N-(6-oxo-1,6-dihydropyridazin-3-yl)nicotinamide |
| CPD-78 | 2-((6-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-2- |
| | oxoethyl)amino)spiro[3.3]heptan-2-yl)amino)-N-(6-(dimethylamino)pyridazin-3- |
| | yl)-6-(methylamino)nicotinamide |
| CPD-79 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(6-(dimethylamino)pyridazin-3-yl)-6- |
| | (methylamino)nicotinamide |
| CPD-80 | 2-((3-((3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)methyl)cyclobutyl)amino)-N-(6-(dimethylamino)pyridazin-3-yl)-6- |
| | (methylamino)nicotinamide |
| CPD-81 | 4-(6-((6-acetyl-8-cyclopentyl-5-methyl-7-oxo-7,8-dihydropyrido[2,3-d]pyrimidin- |
| | 2-yl)amino)pyridin-3-yl)-N-((6-((3-((6-(dimethylamino)pyridazin-3-yl)carbamoyl)- |
| | 6-(methylamino)pyridin-2-yl)amino)spiro[3.3]heptan-2-yl)methyl)piperazine-1- |
| | carboxamide |
| CPD-82 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-3-oxopropyl)spiro[3.3]heptan- |
| | 2-yl)amino)-N-(6-(dimethylamino)pyridazin-3-yl)-6-(methylamino)nicotinamide |
| CPD-83 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propyl)spiro[3.3]heptan-2- |
| | yl)amino)-N-(6-(dimethylamino)pyridazin-3-yl)-6-(methylamino)nicotinamide |
| CPD-84 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-2-oxoethoxy)spiro[3.3]heptan- |
| | 2-yl)amino)-N-(6-(dimethylamino)pyridazin-3-yl)-6-(methylamino)nicotinamide |
| CPD-85 | 2-((5-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)pentyl)amino)-6- |
| | (tert-butylamino)-N-(6-(dimethylamino)pyridazin-3-yl)nicotinamide |
| CPD-86 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(2-cyclopropylpyridin-4-yl)-6-(methylamino)nicotinamide |
| CPD-87 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-cyclopropyl-5-(trifluoromethyl)pyridin-2-yl)-6- |
| | (methylamino)nicotinamide |
| CPD-88 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-(dimethylamino)pyridin-2-yl)-6-(methylamino)nicotinamide |
| CPD-89 | 2-((2-(1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)piperidin-4- |
| | yl)ethyl)amino)-N-(6-(dimethylamino)pyridazin-3-yl)-6- |
| | (methylamino)nicotinamide |
| CPD-90 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(6-cyanopyridin-2-yl)-6-(methylamino)nicotinamide |
| CPD-91 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-6-(methylamino)-N-(6-(trifluoromethyl)pyridin-2-yl)nicotinamide |
| CPD-92 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(5-methyl-1H-pyrazol-3-yl)-6-(methylamino)nicotinamide |
| CPD-93 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(1-methyl-1H-pyrazol-3-yl)-6-(methylamino)nicotinamide |
| CPD-94 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(1-methyl-1H-pyrazol-4-yl)-6-(methylamino)nicotinamide |
| CPD-95 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(5-isopropyl-1-methyl-1H-pyrazol-3-yl)-6-(methylamino)nicotinamide |
| CPD-96 | 2-((6-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)ethyl)amino)spiro[3.3]heptan- |
| | 2-yl)amino)-N-(6-(dimethylamino)pyridazin-3-yl)-6-(methylamino)nicotinamide |
| CPD-97 | 2-((2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)-2-azaspiro[3.3]heptan- |
| | 6-yl)amino)-N-(6-(dimethylamino)pyridazin-3-yl)-6-(methylamino)nicotinamide |
| CPD-98 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(5-cyclopropyl-1-methyl-1H-pyrazol-3-yl)-6- |
| | (methylamino)nicotinamide |
| CPD-99 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan-2- |
| | yl)amino)-N-(1-isopropyl-5-methyl-1H-pyrazol-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 100 | d]pyrimidin-2-yl)amino)pyridin-3-yl)piperazin-1-yl)acetamido)spiro[3.3]heptan-2- |
| | yl)amino)-6-(methylamino)-N-(1H-pyrazol-3-yl)nicotinamide |
| CPD- | 2-((3-(2-((2-(4-(6-((6-acetyl-8-cyclopentyl-5-methyl-7-oxo-7,8-dihydropyrido[2,3- |
| 101 | d]pyrimidin-2-yl)amino)pyridin-3-yl)piperazin-1-yl)ethyl)amino)-2- |
| | oxoethyl)cyclobutyl)amino)-N-(6-(dimethylamino)pyridazin-3-yl)-6- |
| | (methylamino)nicotinamide |
| CPD- | 2-((3-(1-(2-(4-(6-((6-acetyl-8-cyclopentyl-5-methyl-7-oxo-7,8-dihydropyrido[2,3- |
| 102 | d]pyrimidin-2-yl)amino)pyridin-3-yl)piperazin-1-yl)acetyl)azetidin-3- |
| | yl)propyl)amino)-N-(6-(dimethylamino)pyridazin-3-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 103 | d]pyrimidin-2-yl)amino)pyridin-3-yl)piperazin-1-yl)propanoyl)-2- |
| | azaspiro[3.3]heptan-6-yl)amino)-N-(6-cyclopropylpyridin-2-yl)-6- |
| | (methylamino)nicotinamide |
| CPD- | 2-((5-(2-(4-(6-((6-acetyl-8-cyclopentyl-5-methyl-7-oxo-7,8-dihydropyrido[2,3- |
| 104 | d]pyrimidin-2-yl)amino)pyridin-3-yl)piperazin-1-yl)acetamido)pentyl)amino)-N-(6- |
| | aminopyridazin-3-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 105 | d]pyrimidin-2-yl)amino)pyridin-3-yl)piperazin-1-yl)acetamido)pentyl)amino)-6- |
| | (methylamino)-N-(6-methylpyridazin-3-yl)nicotinamide |
| CPD- | 2-((1-(2-(2-(4-(6-((6-acetyl-8-cyclopentyl-5-methyl-7-oxo-7,8-dihydropyrido[2,3- |
| 106 | d]pyrimidin-2-yl)amino)pyridin-3-yl)piperazin-1-yl)acetamido)ethyl)pyrrolidin-3- |
| | yl)amino)-N-(6-(dimethylamino)pyridazin-3-yl)-6-(methylamino)nicotinamide |
| CPD- | 2-((1-(2-(2-(4-(6-((6-acetyl-8-cyclopentyl-5-methyl-7-oxo-7,8-dihydropyrido[2,3- |
| 107 | d]pyrimidin-2-yl)amino)pyridin-3-yl)piperazin-1-yl)acetamido)ethyl)azetidin-3- |
| | yl)amino)-N-(6-(dimethylamino)pyridazin-3-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 108 | d]pyrimidin-2-yl)amino)pyridin-3-yl)piperazin-1-yl)acetamido)pentyl)amino)-N-(6- |
| | methoxypyridazin-3-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 109 | d]pyrimidin-2-yl)amino)pyridin-3-yl)piperazin-1-yl)acetamido)pentyl)amino)-N- |
| | (5,6-dimethylpyridin-2-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 110 | d]pyrimidin-2-yl)amino)pyridin-3-yl)piperazin-1-yl)acetamido)pentyl)amino)-6- |
| | (methylamino)-N-(6-(methylamino)pyridazin-3-yl)nicotinamide |
| CPD- | 2-((5-(2-(4-(6-((6-acetyl-8-cyclopentyl-5-methyl-7-oxo-7,8-dihydropyrido[2,3- |
| 111 | d]pyrimidin-2-yl)amino)pyridin-3-yl)piperazin-1-yl)acetamido)pentyl)amino)-N-(6- |
| | isopropyl-5-methylpyridin-2-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 112 | d]pyrimidin-2-yl)amino)pyridin-3-yl)piperazin-1-yl)acetamido)pentyl)amino)-N-(5- |
| | chloro-6-cyclopropylpyridin-2-yl)-6-(methylamino)nicotinamide |
| CPD- | 2-((5-(2-(4-(6-((6-acetyl-8-cyclopentyl-5-methyl-7-oxo-7,8-dihydropyrido[2,3- |
| 113 | d]pyrimidin-2-yl)amino)pyridin-3-yl)piperazin-1-yl)acetamido)pentyl)amino)-N-(6- |
| | cyclopropyl-5-fluoropyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 114 | d]pyrimidin-2-yl)amino)pyridin-3-yl)piperazin-1-yl)acetamido)spiro[3.3]heptan-2- |
| | yl)amino)-N-(6-cyclopropyl-3-fluoropyridin-2-yl)-6-(methylamino)nicotinamide |
| CPD- | 2-((2-(1-((4-(6-((6-acetyl-8-cyclopentyl-5-methyl-7-oxo-7,8-dihydropyrido[2,3- |
| 115 | d]pyrimidin-2-yl)amino)pyridin-3-yl)piperazin-1-yl)methyl)cyclopropane-1- |
| | carbonyl)-2-azaspiro[3.3]heptan-6-yl)amino)-N-(6-(dimethylamino)pyridazin-3-yl)- |
| | 6-(methylamino)nicotinamide |
| CPD- | 2-((5-(2-(4-(6-((6-acetyl-8-cyclopentyl-5-methyl-7-oxo-7,8-dihydropyrido[2,3- |
| 116 | d]pyrimidin-2-yl)amino)pyridin-3-yl)piperazin-1-yl)acetamido)pentyl)amino)-N-(6- |
| | methoxy-5-methylpyridin-2-yl)-6-(methylamino)nicotinamide |
| CPD- | 2-((2-(3-(4-(6-((6-acetyl-8-cyclopentyl-5-methyl-7-oxo-7,8-dihydropyrido[2,3- |
| 117 | d]pyrimidin-2-yl)amino)pyridin-3-yl)piperazin-1-yl)-2-methylpropanoyl)-2- |
| | azaspiro[3.3]heptan-6-yl)amino)-N-(6-(dimethylamino)pyridazin-3-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 118 | d]pyrimidin-2-yl)amino)pyridin-3-yl)piperazin-1-yl)-2,2-dimethylpropanoyl)-2- |
| | azaspiro[3.3]heptan-6-yl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 119 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-isopropylnicotinamide |
| CPD- | 2-((6-(2-(4-(6-((6-acetyl-8-cyclopentyl-5-methyl-7-oxo-7,8-dihydropyrido[2,3- |
| 120 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-methylnicotinamide |
| CPD- | 2-((6-(2-(4-(6-((6-acetyl-8-cyclopentyl-5-methyl-7-oxo-7,8-dihydropyrido[2,3- |
| 121 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-(trifluoromethyl)nicotinamide |
| CPD- | 2-((6-(2-(4-(6-((6-acetyl-8-cyclopentyl-5-methyl-7-oxo-7,8-dihydropyrido[2,3- |
| 122 | d]pyrimidin-2-yl)amino)pyridin-3-yl)piperazin-1-yl)acetamido)spiro[3.3]heptan-2- |
| | yl)amino)-6-(methylamino)-N-(6-oxo-1,6-dihydropyridin-2-yl)nicotinamide |
| CPD- | 2-((5-(2-(4-(6-((6-acetyl-8-cyclopentyl-5-methyl-7-oxo-7,8-dihydropyrido[2,3- |
| 123 | d]pyrimidin-2-yl)amino)pyridin-3-yl)piperazin-1-yl)acetamido)pentyl)amino)-N-(5- |
| | methyl-6-oxo-1,6-dihydropyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 124 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-ethylnicotinamide |
| CPD- | 2-((6-(2-(4-(6-((6-acetyl-8-cyclopentyl-5-methyl-7-oxo-7,8-dihydropyrido[2,3- |
| 125 | d]pyrimidin-2-yl)amino)pyridin-3-yl)piperazin-1-yl)acetamido)spiro[3.3]heptan-2- |
| | yl)amino)-N-(6-cyclopropyl-5-methoxypyridin-2-yl)-6-(methylamino)nicotinamide |
| CPD- | 2-(((trans-4-(2-(4-(6-((6-acetyl-8-cyclopentyl-5-methyl-7-oxo-7,8- |
| 126 | dihydropyrido[2,3-d]pyrimidin-2-yl)amino)pyridin-3-yl)piperazin-1- |
| | yl)acetamido)cyclohexyl)methyl)amino)-6-(methylamino)-N-(6-methylpyridin-2- |
| | yl)nicotinamide |
| CPD- | 2-((6-(2-(4-(6-((6-acetyl-8-cyclopentyl-5-methyl-7-oxo-7,8-dihydropyrido[2,3- |
| 127 | d]pyrimidin-2-yl)amino)pyridin-3-yl)piperazin-1-yl)acetamido)spiro[3.3]heptan-2- |
| | yl)amino)-N-(1-methyl-5-(trifluoromethyl)-1H-pyrazol-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 128 | d]pyrimidin-2-yl)amino)pyridin-3-yl)piperazin-1-yl)acetamido)spiro[3.3]heptan-2- |
| | yl)amino)-N-(6-cyclopropyl-5-fluoropyridin-2-yl)-6-(methylamino)nicotinamide |
| CPD- | 2-((2-(3-(4-(6-((6-acetyl-8-cyclopentyl-5-methyl-7-oxo-7,8-dihydropyrido[2,3- |
| 129 | d]pyrimidin-2-yl)amino)pyridin-3-yl)piperazin-1-yl)propanoyl)-2- |
| | azaspiro[3.3]heptan-6-yl)amino)-N-(1,5-dimethyl-1H-pyrazol-3-yl)-6- |
| | (methylamino)nicotinamide |
| CPD- | 2-((4-(2-(4-(6-((6-acetyl-8-cyclopentyl-5-methyl-7-oxo-7,8-dihydropyrido[2,3- |
| 130 | d]pyrimidin-2-yl)amino)pyridin-3-yl)piperazin-1-yl)acetamido)cyclohexyl)amino)- |
| | N-(6-(dimethylamino)pyridazin-3-yl)-6-(methylamino)nicotinamide |
| CPD- | 2-(((trans-4-(2-(4-(6-((6-acetyl-8-cyclopentyl-5-methyl-7-oxo-7,8- |
| 131 | dihydropyrido[2,3-d]pyrimidin-2-yl)amino)pyridin-3-yl)piperazin-1- |
| | yl)acetamido)cyclohexyl)methyl)amino)-N-(1,5-dimethyl-1H-pyrazol-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 132 | d]pyrimidin-2-yl)amino)pyridin-3-yl)piperazin-1-yl)acetamido)spiro[3.3]heptan-2- |
| | yl)amino)-N-(5-cyano-6-cyclopropylpyridin-2-yl)-6-(methylamino)nicotinamide |
| CPD- | 2-((2-(4-(6-((6-acetyl-8-cyclopentyl-5-methyl-7-oxo-7,8-dihydropyrido[2,3- |
| 133 | d]pyrimidin-2-yl)amino)pyridin-3-yl)piperazine-1-carbonyl)-2-azaspiro[3.3]heptan- |
| | 6-yl)amino)-N-(6-(dimethylamino)pyridazin-3-yl)-6-(methylamino)nicotinamide |
| CPD- | 4-(6-((6-acetyl-8-cyclopentyl-5-methyl-7-oxo-7,8-dihydropyrido[2,3-d]pyrimidin- |
| 134 | 2-yl)amino)pyridin-3-yl)-N-(6-((3-((6-(dimethylamino)pyridazin-3-yl)carbamoyl)- |
| | 6-(methylamino)pyridin-2-yl)amino)spiro[3.3]heptan-2-yl)piperazine-1- |
| | carboxamide |
| CPD- | 2-((6-(2-(4-(6-((6-acetyl-8-cyclopentyl-5-methyl-7-oxo-7,8-dihydropyrido[2,3- |
| 135 | d]pyrimidin-2-yl)amino)pyridin-3-yl)piperazin-1-yl)acetamido)spiro[3.3]heptan-2- |
| | yl)amino)-N-(2-cyclopropyl-1-methyl-1H-imidazol-4-yl)-6- |
| | (methylamino)nicotinamide |
| CPD- | 2-((3-(2-(2-(4-(6-((6-acetyl-8-cyclopentyl-5-methyl-7-oxo-7,8-dihydropyrido[2,3- |
| 136 | d]pyrimidin-2-yl)amino)pyridin-3-yl)piperazin-1- |
| | yl)acetamido)ethyl)cyclopentyl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 137 | d]pyrimidin-2-yl)amino)pyridin-3-yl)piperazin-1-yl)acetamido)spiro[3.3]heptan-2- |
| | yl)amino)-6-(methylamino)-N-(pyridin-2-yl)nicotinamide |
| CPD- | 2-((6-(2-(4-(6-((6-acetyl-8-cyclopentyl-5-methyl-7-oxo-7,8-dihydropyrido[2,3- |
| 138 | d]pyrimidin-2-yl)amino)pyridin-3-yl)piperazin-1-yl)acetamido)spiro[3.3]heptan-2- |
| | yl)amino)-6-cyclopropyl-N-(6-cyclopropylpyridin-2-yl)nicotinamide |
| CPD- | 2-((6-(2-(4-(6-((6-acetyl-8-cyclopentyl-5-methyl-7-oxo-7,8-dihydropyrido[2,3- |
| 139 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-(isopropylamino)nicotinamide |
| CPD- | 2-((6-(4-(6-((6-acetyl-8-cyclopentyl-5-methyl-7-oxo-7,8-dihydropyrido[2,3- |
| 140 | d]pyrimidin-2-yl)amino)pyridin-3-yl)piperazine-1-carbonyl)spiro[3.3]heptan-2- |
| | yl)amino)-N-(6-(dimethylamino)pyridazin-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 141 | d]pyrimidin-2-yl)amino)pyridin-3-yl)piperazin-1-yl)acetamido)spiro[3.3]heptan-2- |
| | yl)amino)-6-cyano-N-(6-cyclopropylpyridin-2-yl)nicotinamide |
| CPD- | 6-((7-(2-(4-(6-((6-acetyl-8-cyclopentyl-5-methyl-7-oxo-7,8-dihydropyrido[2,3- |
| 142 | d]pyrimidin-2-yl)amino)pyridin-3-yl)piperazin-1-yl)acetamido)heptyl)amino)-6'- |
| | cyano-N-(4,5-dimethylthiazol-2-yl)-[2,3′-bipyridine]-5-carboxamide |
| CPD- | 2-((7-(2-(4-(6-((6-acetyl-8-cyclopentyl-5-methyl-7-oxo-7,8-dihydropyrido[2,3- |
| 143 | d]pyrimidin-2-yl)(methyl)amino)pyridin-3-yl)piperazin-1- |
| | yl)acetamido)heptyl)amino)-N-(6-(dimethylamino)pyridazin-3-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 144 | d]pyrimidin-2-yl)amino)pyridin-3-yl)piperazin-1-yl)propanoyl)-2- |
| | azaspiro[3.3]heptan-6-yl)amino)-N-(1-methyl-1H-pyrazol-3-yl)-6- |
| | (methylamino)nicotinamide |
| CPD- | 2-(((1-(2-(4-(6-((6-acetyl-8-cyclopentyl-5-methyl-7-oxo-7,8-dihydropyrido[2,3- |
| 145 | d]pyrimidin-2-yl)amino)pyridin-3-yl)piperazin-1-yl)acetyl)piperidin-4- |
| | yl)methyl)amino)-N-(6-(dimethylamino)pyridazin-3-yl)-6- |
| | (methylamino)nicotinamide |
| CPD- | 2-(((1-(3-(4-(6-((6-acetyl-8-cyclopentyl-5-methyl-7-oxo-7,8-dihydropyrido[2,3- |
| 146 | d]pyrimidin-2-yl)amino)pyridin-3-yl)piperazin-1-yl)propanoyl)piperidin-4- |
| | yl)methyl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 147 | d]pyrimidin-2-yl)amino)pyridin-3-yl)piperazin-1-yl)-2-oxoethyl)spiro[3.3]heptan-2- |
| | yl)amino)-N-(6-(dimethylamino)pyridazin-3-yl)-6-(methylamino)nicotinamide |
| CPD- | 2-((3-(2-(4-(6-((6-acetyl-8-cyclopentyl-5-methyl-7-oxo-7,8-dihydropyrido[2,3- |
| 148 | d]pyrimidin-2-yl)amino)pyridin-3-yl)piperazin-1-yl)-2-oxoethyl)cyclobutyl)amino)- |
| | N-(6-(dimethylamino)pyridazin-3-yl)-6-(methylamino)nicotinamide |
| CPD- | 2-((3-(2-(4-(6-((6-acetyl-8-cyclopentyl-5-methyl-7-oxo-7,8-dihydropyrido[2,3- |
| 149 | d]pyrimidin-2-yl)amino)pyridin-3-yl)piperazin-1-yl)acetamido)cyclobutyl)amino)- |
| | N-(6-(dimethylamino)pyridazin-3-yl)-6-(methylamino)nicotinamide |
| CPD- | 2-((3-(3-(4-(6-((6-acetyl-8-cyclopentyl-5-methyl-7-oxo-7,8-dihydropyrido[2,3- |
| 150 | d]pyrimidin-2-yl)amino)pyridin-3-yl)piperazin-1- |
| | yl)propanamido)cyclobutyl)amino)-N-(6-(dimethylamino)pyridazin-3-yl)-6- |
| | (methylamino)nicotinamide |
| CPD- | 4-(6-((6-acetyl-8-cyclopentyl-5-methyl-7-oxo-7,8-dihydropyrido[2,3-d]pyrimidin- |
| 151 | 2-yl)amino)pyridin-3-yl)-N-(3-((3-((6-(dimethylamino)pyridazin-3-yl)carbamoyl)- |
| | 6-(methylamino)pyridin-2-yl)amino)cyclobutyl)piperazine-1-carboxamide |
| CPD- | 2-((6-(2-(4-(6-((6-acetyl-8-cyclopentyl-5-methyl-7-oxo-7,8-dihydropyrido[2,3- |
| 152 | d]pyrimidin-2-yl)amino)pyridin-3-yl)piperazin-1-yl)acetamido)spiro[3.3]heptan-2- |
| | yl)amino)-N-(1-methyl-1H-imidazol-4-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 153 | d]pyrimidin-2-yl)amino)pyridin-3-yl)piperazin-1-yl)acetamido)spiro[3.3]heptan-2- |
| | yl)amino)-N-(1-methyl-1H-pyrazol-5-yl)-6-(methylamino)nicotinamide |
| CPD- | 2-(((trans-4-(2-(4-(6-((6-acetyl-8-cyclopentyl-5-methyl-7-oxo-7,8- |
| 154 | dihydropyrido[2,3-d]pyrimidin-2-yl)amino)pyridin-3-yl)piperazin-1- |
| | yl)acetamido)cyclohexyl)methyl)amino)-N-(6-cyclopropylpyridin-2-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 155 | d]pyrimidin-2-yl)amino)pyridin-3-yl)piperazin-1-yl)propanoyl)-2- |
| | azaspiro[3.3]heptan-6-yl)amino)-6-(methylamino)-N-(6-methylpyridin-2- |
| | yl)nicotinamide |
| CPD- | 2-((3-(4-(4-(6-((6-acetyl-8-cyclopentyl-5-methyl-7-oxo-7,8-dihydropyrido[2,3- |
| 156 | d]pyrimidin-2-yl)amino)pyridin-3-yl)piperazin-1-yl)-4- |
| | oxobutyl)cyclobutyl)amino)-N-(6-(dimethylamino)pyridazin-3-yl)-6- |
| | (methylamino)nicotinamide |
| CPD- | 2-((3-((2-(4-(6-((6-acetyl-8-cyclopentyl-5-methyl-7-oxo-7,8-dihydropyrido[2,3- |
| 157 | d]pyrimidin-2-yl)amino)pyridin-3-yl)piperazin-1- |
| | yl)ethyl)carbamoyl)cyclobutyl)amino)-N-(6-(dimethylamino)pyridazin-3-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 158 | d]pyrimidin-2-yl)amino)pyridin-3-yl)piperazin-1-yl)propanoyl)-2- |
| | azaspiro[3.4]octan-6-yl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 159 | d]pyrimidin-2-yl)amino)pyridin-3-yl)piperazin-1-yl)acetamido)spiro[3.3]heptan-2- |
| | yl)amino)-N-(isoxazol-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 160 | d]pyrimidin-2-yl)amino)pyridin-3-yl)piperazin-1-yl)acetamido)spiro[3.3]heptan-2- |
| | yl)amino)-6-(methylamino)-N-(5-methylisoxazol-3-yl)nicotinamide |
| CPD- | 2-((6-(2-(4-(6-((6-acetyl-8-cyclopentyl-5-methyl-7-oxo-7,8-dihydropyrido[2,3- |
| 161 | d]pyrimidin-2-yl)amino)pyridin-3-yl)piperazin-1-yl)acetamido)spiro[3.3]heptan-2- |
| | yl)amino)-N-(6-methoxypyridin-2-yl)-6-(methylamino)nicotinamide |
| CPD- | 2-((3-((2-(4-(6-((6-acetyl-8-cyclopentyl-5-methyl-7-oxo-7,8-dihydropyrido[2,3- |
| 162 | d]pyrimidin-2-yl)amino)pyridin-3-yl)piperazin-1- |
| | yl)acetamido)methyl)cyclobutyl)amino)-N-(6-cyclopropylpyridin-2-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 163 | d]pyrimidin-2-yl)amino)pyridin-3-yl)piperazin-1-yl)acetamido)spiro[3.3]heptan-2- |
| | yl)amino)-N-(6-cyclopropylpyridin-2-yl)-6-((methyl-d3)amino)nicotinamide |
| CPD- | 2-((3-((2-(4-(6-((6-acetyl-8-cyclopentyl-5-methyl-7-oxo-7,8-dihydropyrido[2,3- |
| 164 | d]pyrimidin-2-yl)amino)pyridin-3-yl)piperazin-1-yl)-2- |
| | oxoethoxy)methyl)cyclobutyl)amino)-N-(6-(dimethylamino)pyridazin-3-yl)-6- |
| | (methylamino)nicotinamide |
| CPD- | 2-((3-((2-(4-(6-((6-acetyl-8-cyclopentyl-5-methyl-7-oxo-7,8-dihydropyrido[2,3- |
| 165 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | (methylamino)nicotinamide |
| CPD- | 4-(6-((6-acetyl-8-cyclopentyl-5-methyl-7-oxo-7,8-dihydropyrido[2,3-d]pyrimidin- |
| 166 | 2-yl)amino)pyridin-3-yl)-N-((3-((3-((6-(dimethylamino)pyridazin-3-yl)carbamoyl)- |
| | 6-(methylamino)pyridin-2-yl)amino)cyclobutyl)methyl)piperazine-1-carboxamide |
| CPD- | 2-((3-((2-(4-(6-((6-acetyl-8-cyclopentyl-5-methyl-7-oxo-7,8-dihydropyrido[2,3- |
| 167 | d]pyrimidin-2-yl)amino)pyridin-3-yl)piperazin-1-yl)-2- |
| | methylpropanamido)methyl)cyclobutyl)amino)-N-(6-(dimethylamino)pyridazin-3- |
| | yl)-6-(methylamino)nicotinamide |
| CPD- | 2-((3-(2-(4-(6-((6-acetyl-8-cyclopentyl-5-methyl-7-oxo-7,8-dihydropyrido[2,3- |
| 168 | d]pyrimidin-2-yl)amino)pyridin-3-yl)piperazin-1-yl)-2- |
| | oxoethoxy)cyclobutyl)amino)-N-(6-(dimethylamino)pyridazin-3-yl)-6- |
| | (methylamino)nicotinamide |
| CPD- | 4-(6-((6-acetyl-8-cyclopentyl-5-methyl-7-oxo-7,8-dihydropyrido[2,3-d]pyrimidin- |
| 169 | 2-yl)amino)pyridin-3-yl)-N-((trans-4-(((3-((6-(dimethylamino)pyridazin-3- |
| | yl)carbamoyl)-6-(methylamino)pyridin-2- |
| | yl)amino)methyl)cyclohexyl)methyl)piperazine-1-carboxamide |
| CPD- | 2-((3-((2-(4-(6-((6-acetyl-8-cyclopentyl-5-methyl-7-oxo-7,8-dihydropyrido[2,3- |
| 170 | d]pyrimidin-2-yl)amino)pyridin-3-yl)piperazin-1- |
| | yl)acetamido)methyl)cyclobutyl)amino)-6-(methylamino)-N-(6-methylpyridin-2- |
| | yl)nicotinamide |
| CPD- | 2-((2-(1-((4-(6-((6-acetyl-8-cyclopentyl-5-methyl-7-oxo-7,8-dihydropyrido[2,3- |
| 171 | d]pyrimidin-2-yl)amino)pyridin-3-yl)piperazin-1-yl)methyl)cyclopropane-1- |
| | carbonyl)-2-azaspiro[3.3]heptan-6-yl)amino)-6-(methylamino)-N-(6-methylpyridin- |
| | 2-yl)nicotinamide |
| CPD- | 2-((3-((1-((4-(6-((6-acetyl-8-cyclopentyl-5-methyl-7-oxo-7,8-dihydropyrido[2,3- |
| 172 | d]pyrimidin-2-yl)amino)pyridin-3-yl)piperazin-1-yl)methyl)cyclopropane-1- |
| | carboxamido)methyl)cyclobutyl)amino)-N-(6-(dimethylamino)pyridazin-3-yl)-6- |
| | (methylamino)nicotinamide |
| CPD- | 2-((2-(1-((4-(6-((6-acetyl-8-cyclopentyl-5-methyl-7-oxo-7,8-dihydropyrido[2,3- |
| 173 | d]pyrimidin-2-yl)amino)pyridin-3-yl)piperazin-1-yl)methyl)cyclopropane-1- |
| | carbonyl)-2-azaspiro[3.3]heptan-6-yl)amino)-N-(6-cyclopropylpyridin-2-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 174 | d]pyrimidin-2-yl)amino)pyridin-3-yl)piperazin-1-yl)-2- |
| | methylpropanamido)spiro[3.3]heptan-2-yl)amino)-N-(6-(dimethylamino)pyridazin- |
| | 3-yl)-6-(methylamino)nicotinamide |
| CPD- | 2-((2-(2-(4-(6-((6-acetyl-8-cyclopentyl-5-methyl-7-oxo-7,8-dihydropyrido[2,3- |
| 175 | d]pyrimidin-2-yl)amino)pyridin-3-yl)piperazin-1-yl)-2-methylpropanoyl)-2- |
| | azaspiro[3.3]heptan-6-yl)amino)-N-(6-cyclopropylpyridin-2-yl)-6- |
| | (methylamino)nicotinamide |
| CPD- | 2-((2-(2-(4-(6-((6-acetyl-8-cyclopentyl-5-methyl-7-oxo-7,8-dihydropyrido[2,3- |
| 176 | d]pyrimidin-2-yl)amino)pyridin-3-yl)piperazin-1-yl)-2-methylpropanoyl)-2- |
| | azaspiro[3.3]heptan-6-yl)amino)-6-(methylamino)-N-(6-methylpyridin-2- |
| | yl)nicotinamide |
| CPD- | 2-((6-(2-(4-(6-((6-acetyl-8-cyclopentyl-5-methyl-7-oxo-7,8-dihydropyrido[2,3- |
| 177 | d]pyrimidin-2-yl)amino)pyridin-3-yl)piperazin-1-yl)acetamido)spiro[3.3]heptan-2- |
| | yl)amino)-N-(6-cyclopropyl-5-(dimethylamino)pyridin-2-yl)-6- |
| | (methylamino)nicotinamide |
| CPD- | 2-((3-(3-(4-(6-((6-acetyl-8-cyclopentyl-5-methyl-7-oxo-7,8-dihydropyrido[2,3- |
| 178 | d]pyrimidin-2-yl)amino)pyridin-3-yl)piperazin-1-yl)-3- |
| | oxopropoxy)cyclobutyl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 179 | d]pyrimidin-2-yl)amino)pyridin-3-yl)piperazin-1-yl)acetamido)spiro[3.3]heptan-2- |
| | yl)amino)-N-(1,2-dimethyl-1H-imidazol-4-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 180 | d]pyrimidin-2-yl)amino)pyridin-3-yl)piperazin-1-yl)acetamido)spiro[3.3]heptan-2- |
| | yl)amino)-N-(6-aminopyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 181 | d]pyrimidin-2-yl)amino)pyridin-3-yl)piperazin-1-yl)acetamido)spiro[3.3]heptan-2- |
| | yl)amino)-6-(methylamino)-N-(6-(methylamino)pyridin-2-yl)nicotinamide |
| CPD- | 2-((6-(2-(4-(6-((6-acetyl-8-cyclopentyl-5-methyl-7-oxo-7,8-dihydropyrido[2,3- |
| 182 | d]pyrimidin-2-yl)amino)pyridin-3-yl)piperazin-1-yl)acetamido)spiro[3.3]heptan-2- |
| | yl)amino)-N-(5,6-dimethylpyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 183 | d]pyrimidin-2-yl)amino)pyridin-3-yl)piperazin-1-yl)acetamido)spiro[3.3]heptan-2- |
| | yl)amino)-N-(1-methyl-1H-pyrrol-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 184 | d]pyrimidin-2-yl)amino)pyridin-3-yl)piperazin-1-yl)acetamido)spiro[3.3]heptan-2- |
| | yl)amino)-N-(6-(difluoromethyl)pyridin-2-yl)-6-(methylamino)nicotinamide |
| CPD- | 2-((3-(3-(4-(6-((6-acetyl-8-cyclopentyl-5-methyl-7-oxo-7,8-dihydropyrido[2,3- |
| 185 | d]pyrimidin-2-yl)amino)pyridin-3-yl)piperazin-1-yl)-3- |
| | oxopropyl)cyclobutyl)amino)-N-(6-(dimethylamino)pyridazin-3-yl)-6- |
| | (methylamino)nicotinamide |
| CPD- | 2-((1-(3-(4-(6-((6-acetyl-8-cyclopentyl-5-methyl-7-oxo-7,8-dihydropyrido[2,3- |
| 186 | d]pyrimidin-2-yl)amino)pyridin-3-yl)piperazin-1-yl)propanoyl)azetidin-3- |
| | yl)amino)-N-(6-(dimethylamino)pyridazin-3-yl)-6-(methylamino)nicotinamide |
| CPD- | 2-((1-(2-(4-(6-((6-acetyl-8-cyclopentyl-5-methyl-7-oxo-7,8-dihydropyrido[2,3- |
| 187 | d]pyrimidin-2-yl)amino)pyridin-3-yl)piperazin-1-yl)acetyl)azetidin-3-yl)amino)-N- |
| | (6-(dimethylamino)pyridazin-3-yl)-6-(methylamino)nicotinamide |
| CPD- | 2-(((3-((2-(4-(6-((6-acetyl-8-cyclopentyl-5-methyl-7-oxo-7,8-dihydropyrido[2,3- |
| 188 | d]pyrimidin-2-yl)amino)pyridin-3-yl)piperazin-1- |
| | yl)acetamido)methyl)cyclobutyl)methyl)amino)-N-(6-(dimethylamino)pyridazin-3- |
| | yl)-6-(methylamino)nicotinamide |
| CPD- | 2-((6-((1-(4-(6-((6-acetyl-8-cyclopentyl-5-methyl-7-oxo-7,8-dihydropyrido[2,3- |
| 189 | d]pyrimidin-2-yl)amino)pyridin-3-yl)piperazin-1-yl)-1-oxopropan-2- |
| | yl)oxy)spiro[3.3]heptan-2-yl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 190 | d]pyrimidin-2-yl)amino)pyridin-3-yl)piperazin-1-yl)acetamido)spiro[3.3]heptan-2- |
| | yl)amino)-6-(isopropylamino)-N-(6-methylpyridin-2-yl)nicotinamide |
| CPD- | 2-((6-(2-(4-(6-((6-acetyl-8-cyclopentyl-5-methyl-7-oxo-7,8-dihydropyrido[2,3- |
| 191 | d]pyrimidin-2-yl)amino)pyridin-3-yl)piperazin-1-yl)acetamido)spiro[3.3]heptan-2- |
| | yl)amino)-6-methyl-N-(6-methylpyridin-2-yl)nicotinamide |
| CPD- | 2-((6-(2-(4-(6-((6-acetyl-8-cyclopentyl-5-methyl-7-oxo-7,8-dihydropyrido[2,3- |
| 192 | d]pyrimidin-2-yl)amino)pyridin-3-yl)piperazin-1-yl)acetamido)spiro[3.3]heptan-2- |
| | yl)amino)-6-methyl-N-(5-methylisoxazol-3-yl)nicotinamide |
| CPD- | 2-(((cis-4-(2-(4-(6-((6-acetyl-8-cyclopentyl-5-methyl-7-oxo-7,8-dihydropyrido[2,3- |
| 193 | d]pyrimidin-2-yl)amino)pyridin-3-yl)piperazin-1- |
| | yl)acetamido)cyclohexyl)methyl)amino)-N-(6-(dimethylamino)pyridazin-3-yl)-6- |
| | (methylamino)nicotinamide |
| CPD- | 2-(2-(4-(6-((6-acetyl-8-cyclopentyl-5-methyl-7-oxo-7,8-dihydropyrido[2,3- |
| 194 | d]pyrimidin-2-yl)amino)pyridin-3-yl)piperazin-1-yl)acetamido)-N-(6- |
| | (dimethylamino)pyridazin-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 195 | d]pyrimidin-2-yl)amino)pyridin-3-yl)piperazin-1-yl)acetamido)spiro[3.3]heptan-2- |
| | yl)amino)-N-(2-isopropyl-1-methyl-1H-imidazol-4-yl)-6- |
| | (methylamino)nicotinamide |
| CPD- | 2-((3-(2-(4-(6-((6-acetyl-8-cyclopentyl-5-methyl-7-oxo-7,8-dihydropyrido[2,3- |
| 196 | d]pyrimidin-2-yl)amino)pyridin-3-yl)piperazin-1-yl)acetamido)cyclobutyl)amino)- |
| | 6-(methylamino)-N-(6-methylpyridin-2-yl)nicotinamide |
| CPD- | 2-((3-(2-(4-(6-((6-acetyl-8-cyclopentyl-5-methyl-7-oxo-7,8-dihydropyrido[2,3- |
| 197 | d]pyrimidin-2-yl)amino)pyridin-3-yl)piperazin-1-yl)acetamido)cyclobutyl)amino)- |
| | N-(1,5-dimethyl-1H-pyrazol-3-yl)-6-(methylamino)nicotinamide |
| CPD- | 2-((3-(2-(4-(6-((6-acetyl-8-cyclopentyl-5-methyl-7-oxo-7,8-dihydropyrido[2,3- |
| 198 | d]pyrimidin-2-yl)amino)pyridin-3-yl)piperazin-1-yl)acetamido)cyclobutyl)amino)- |
| | 6-(methylamino)-N-(5-methylisoxazol-3-yl)nicotinamide |
| CPD- | 2-((6-(2-(4-(6-((6-acetyl-8-cyclopentyl-5-methyl-7-oxo-7,8-dihydropyrido[2,3- |
| 199 | d]pyrimidin-2-yl)amino)pyridin-3-yl)piperazin-1-yl)acetamido)spiro[3.3]heptan-2- |
| | yl)amino)-6-(isopropylamino)-N-(5-methylisoxazol-3-yl)nicotinamide |
| CPD- | 2-((1-(2-(4-(6-((6-acetyl-8-cyclopentyl-5-methyl-7-oxo-7,8-dihydropyrido[2,3- |
| 200 | d]pyrimidin-2-yl)amino)pyridin-3-yl)piperazin-1-yl)acetyl)piperidin-4-yl)amino)- |
| | N-(6-(dimethylamino)pyridazin-3-yl)-6-(methylamino)nicotinamide |
| CPD- | 2-((1-(2-(4-(6-((6-acetyl-8-cyclopentyl-5-methyl-7-oxo-7,8-dihydropyrido[2,3- |
| 201 | d]pyrimidin-2-yl)amino)pyridin-3-yl)piperazin-1-yl)acetyl)pyrrolidin-3-yl)amino)- |
| | N-(6-(dimethylamino)pyridazin-3-yl)-6-(methylamino)nicotinamide |
| CPD- | 2-((3-((4-((4-(6-((6-acetyl-8-cyclopentyl-5-methyl-7-oxo-7,8-dihydropyrido[2,3- |
| 202 | d]pyrimidin-2-yl)amino)pyridin-3-yl)piperazin-1-yl)methyl)-1H-1,2,3-triazol-1- |
| | yl)methyl)cyclobutyl)amino)-N-(6-(dimethylamino)pyridazin-3-yl)-6- |
| | (methylamino)nicotinamide |
| CPD- | 2-((1-(3-(4-(6-((6-acetyl-8-cyclopentyl-5-methyl-7-oxo-7,8-dihydropyrido[2,3- |
| 203 | d]pyrimidin-2-yl)amino)pyridin-3-yl)piperazin-1-yl)propanoyl)piperidin-4- |
| | yl)amino)-N-(6-(dimethylamino)pyridazin-3-yl)-6-(methylamino)nicotinamide |
| CPD- | 2-(((trans-4-(3-(4-(6-((6-acetyl-8-cyclopentyl-5-methyl-7-oxo-7,8- |
| 204 | dihydropyrido[2,3-d]pyrimidin-2-yl)amino)pyridin-3-yl)piperazin-1-yl)-3- |
| | oxopropyl)cyclohexyl)methyl)amino)-N-(6-(dimethylamino)pyridazin-3-yl)-6- |
| | (methylamino)nicotinamide |
| CPD- | 2-((3-((2-(4-(6-((6-acetyl-8-cyclopentyl-5-methyl-7-oxo-7,8-dihydropyrido[2,3- |
| 205 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | (isopropylamino)nicotinamide |
| CPD- | 2-((3-((2-(4-(6-((6-acetyl-8-cyclopentyl-5-methyl-7-oxo-7,8-dihydropyrido[2,3- |
| 206 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | ((methyl-d₃)amino)nicotinamide |
| CPD- | 2-((6-(2-(4-(6-((6-acetyl-8-cyclopentyl-5-methyl-7-oxo-7,8-dihydropyrido[2,3- |
| 207 | d]pyrimidin-2-yl)amino)pyridin-3-yl)piperazin-1-yl)acetamido)spiro[3.3]heptan-2- |
| | yl)amino)-N-(2-cyclopropyl-1-methyl-1H-imidazol-4-yl)-6-methylnicotinamide |
| CPD- | 2-((2-(3-(4-(6-((6-acetyl-8-cyclopentyl-5-methyl-7-oxo-7,8-dihydropyrido[2,3- |
| 208 | d]pyrimidin-2-yl)amino)pyridin-3-yl)piperazin-1-yl)-2,2-difluoropropanoyl)-2- |
| | azaspiro[3.3]heptan-6-yl)amino)-N-(6-(dimethylamino)pyridazin-3-yl)-6- |
| | (methylamino)nicotinamide |
| CPD- | 2-((3-((2-(4-(6-((6-acetyl-8-cyclopentyl-5-methyl-7-oxo-7,8-dihydropyrido[2,3- |
| 209 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | methylnicotinamide |
| CPD- | 2-((3-((2-(4-(6-((6-acetyl-8-cyclopentyl-5-methyl-7-oxo-7,8-dihydropyrido[2,3- |
| 210 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | ethylnicotinamide |
| CPD- | 2-((1-(4-(4-(6-((6-acetyl-8-cyclopentyl-5-methyl-7-oxo-7,8-dihydropyrido[2,3- |
| 211 | d]pyrimidin-2-yl)amino)pyridin-3-yl)piperazin-1-yl)butanoyl)azetidin-3-yl)amino)- |
| | N-(6-(dimethylamino)pyridazin-3-yl)-6-(methylamino)nicotinamide |
| CPD- | 2-((3-((2-(4-(6-((6-acetyl-8-cyclopentyl-5-methyl-7-oxo-7,8-dihydropyrido[2,3- |
| 212 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-6- |
| | isopropylnicotinamide |
| CPD- | 2-((3-((2-(4-(6-((6-acetyl-8-cyclopentyl-5-methyl-7-oxo-7,8-dihydropyrido[2,3- |
| 213 | d]pyrimidin-2-yl)amino)pyridin-3-yl)piperazin-1- |
| | yl)acetamido)methyl)cyclobutyl)amino)-6-cyclopropyl-N-(1,5-dimethyl-1H- |
| | pyrazol-3-yl)nicotinamide |
| CPD- | 2-(((4-(2-(4-(6-((6-acetyl-8-cyclopentyl-5-methyl-7-oxo-7,8-dihydropyrido[2,3- |
| 214 | d]pyrimidin-2-yl)amino)pyridin-3-yl)piperazin-1-yl)-2- |
| | oxoethoxy)cyclohexyl)methyl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 215 | d]pyrimidin-2-yl)amino)pyridin-3-yl)piperazin-1-yl)acetamido)spiro[3.3]heptan-2- |
| | yl)amino)-N-(5-cyclopropylisoxazol-3-yl)-6-(methylamino)nicotinamide |
| CPD- | 2-((6-((1-(4-(6-((6-acetyl-8-cyclopentyl-5-methyl-7-oxo-7,8-dihydropyrido[2,3- |
| 216 | d]pyrimidin-2-yl)amino)pyridin-3-yl)piperazin-1-yl)-2-methyl-1-oxopropan-2- |
| | yl)oxy)spiro[3.3]heptan-2-yl)amino)-N-(6-(dimethylamino)pyridazin-3-yl)-6- |
| | (methylamino)nicotinamide |
| CPD- | 2-((3-(2-(4-(6-((6-acetyl-8-cyclopentyl-5-methyl-7-oxo-7,8-dihydropyrido[2,3- |
| 217 | d]pyrimidin-2-yl)amino)pyridin-3-yl)piperazin-1-yl)acetamido)cyclobutyl)amino)- |
| | N-(2-cyclopropyl-1-methyl-1H-imidazol-4-yl)-6-(methylamino)nicotinamide |
| CPD- | 2-((1-(3-(4-(6-((6-acetyl-8-cyclopentyl-5-methyl-7-oxo-7,8-dihydropyrido[2,3- |
| 218 | d]pyrimidin-2-yl)amino)pyridin-3-yl)piperazin-1-yl)propanoyl)pyrrolidin-3- |
| | yl)amino)-N-(6-(dimethylamino)pyridazin-3-yl)-6-(methylamino)nicotinamide |
| CPD- | 2-((3-(2-(4-(6-((6-acetyl-8-cyclopentyl-5-methyl-7-oxo-7,8-dihydropyrido[2,3- |
| 219 | d]pyrimidin-2-yl)amino)pyridin-3-yl)piperazin-1-yl)acetamido)cyclopentyl)amino)- |
| | N-(6-(dimethylamino)pyridazin-3-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 220 | d]pyrimidin-2-yl)amino)pyridin-3-yl)piperazin-1-yl)acetamido)spiro[3.3]heptan-2- |
| | yl)amino)-N-(5-methoxy-6-methylpyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 221 | d]pyrimidin-2-yl)amino)pyridin-3-yl)piperazin-1-yl)acetamido)spiro[3.3]heptan-2- |
| | yl)amino)-N-(5-fluoro-6-methylpyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 222 | d]pyrimidin-2-yl)amino)pyridin-3-yl)piperazin-1-yl)acetamido)spiro[3.3]heptan-2- |
| | yl)amino)-N-(2-cyclopropyl-1-methyl-1H-imidazol-4-yl)-6- |
| | (isopropylamino)nicotinamide |
| CPD- | 2-((trans-4-(3-(4-(6-((6-acetyl-8-cyclopentyl-5-methyl-7-oxo-7,8- |
| 223 | dihydropyrido[2,3-d]pyrimidin-2-yl)amino)pyridin-3-yl)piperazin-1- |
| | yl)propanamido)cyclohexyl)amino)-N-(6-(dimethylamino)pyridazin-3-yl)-6- |
| | (methylamino)nicotinamide |
| CPD- | 2-((2-(3-(4-(6-((6-acetyl-8-cyclopentyl-5-methyl-7-oxo-7,8-dihydropyrido[2,3- |
| 224 | d]pyrimidin-2-yl)amino)pyridin-3-yl)piperazin-1-yl)butanoyl)-2- |
| | azaspiro[3.3]heptan-6-yl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 225 | d]pyrimidin-2-yl)amino)pyridin-3-yl)piperazin-1-yl)acetamido)spiro[3.3]heptan-2- |
| | yl)amino)-6-((methyl-d₃)amino)-N-(5-methylisoxazol-3-yl)nicotinamide |
| CPD- | 2-((6-((1-(4-(6-((6-acetyl-8-cyclopentyl-5-methyl-7-oxo-7,8-dihydropyrido[2,3- |
| 226 | d]pyrimidin-2-yl)amino)pyridin-3-yl)piperazin-1-yl)-2-methyl-1-oxopropan-2- |
| | yl)oxy)spiro[3.3]heptan-2-yl)amino)-6-((methyl-d₃)amino)-N-(6-methylpyridin-2- |
| | yl)nicotinamide |
| CPD- | 2-((6-(2-(4-(6-((6-acetyl-8-cyclopentyl-5-methyl-7-oxo-7,8-dihydropyrido[2,3- |
| 227 | d]pyrimidin-2-yl)amino)pyridin-3-yl)piperazin-1-yl)acetamido)spiro[3.3]heptan-2- |
| | yl)amino)-N-(5-isopropylisoxazol-3-yl)-6-(methylamino)nicotinamide |
| CPD- | 2-((1-(2-(4-(6-((6-acetyl-8-cyclopentyl-5-methyl-7-oxo-7,8-dihydropyrido[2,3- |
| 228 | d]pyrimidin-2-yl)amino)pyridin-3-yl)piperazin-1-yl)acetyl)piperidin-4-yl)amino)- |
| | N-(1,5-dimethyl-1H-pyrazol-3-yl)-6-(methylamino)nicotinamide |
| CPD- | 2-((1-(2-(4-(6-((6-acetyl-8-cyclopentyl-5-methyl-7-oxo-7,8-dihydropyrido[2,3- |
| 229 | d]pyrimidin-2-yl)amino)pyridin-3-yl)piperazin-1-yl)acetyl)piperidin-4-yl)amino)-6- |
| | (methylamino)-N-(5-methylisoxazol-3-yl)nicotinamide |
| CPD- | 2-((1-(2-(4-(6-((6-acetyl-8-cyclopentyl-5-methyl-7-oxo-7,8-dihydropyrido[2,3- |
| 230 | d]pyrimidin-2-yl)amino)pyridin-3-yl)piperazin-1-yl)acetyl)piperidin-4-yl)amino)- |
| | N-(1,5-dimethyl-1H-pyrazol-3-yl)-6-((methyl-d₃)amino)nicotinamide |
| CPD- | 2-((1-(2-(4-(6-((6-acetyl-8-cyclopentyl-5-methyl-7-oxo-7,8-dihydropyrido[2,3- |
| 231 | d]pyrimidin-2-yl)amino)pyridin-3-yl)piperazin-1-yl)acetyl)piperidin-4-yl)amino)-6- |
| | ((methyl-d₃)amino)-N-(5-methylisoxazol-3-yl)nicotinamide |
| CPD- | 2-((6-(2-(4-(6-((6-acetyl-8-cyclopentyl-5-methyl-7-oxo-7,8-dihydropyrido[2,3- |
| 232 | d]pyrimidin-2-yl)amino)pyridin-3-yl)piperazin-1-yl)acetamido)spiro[3.3]heptan-2- |
| | yl)amino)-6-((methyl-d₃)amino)-N-(6-methylpyridin-2-yl)nicotinamide |
| CPD- | 2-((1-(3-(4-(6-((6-acetyl-8-cyclopentyl-5-methyl-7-oxo-7,8-dihydropyrido[2,3- |
| 233 | d]pyrimidin-2-yl)amino)pyridin-3-yl)piperazin-1-yl)-2,2- |
| | difluoropropanoyl)azetidin-3-yl)amino)-N-(1,5-dimethyl-1H-pyrazol-3-yl)-6- |
| | ((methyl-d₃)amino)nicotinamide |
| CPD- | 2-((1-(3-(4-(6-((6-acetyl-8-cyclopentyl-5-methyl-7-oxo-7,8-dihydropyrido[2,3- |
| 234 | d]pyrimidin-2-yl)amino)pyridin-3-yl)piperazin-1-yl)-2,2- |
| | difluoropropanoyl)azetidin-3-yl)amino)-6-((methyl-d₃)amino)-N-(5-methylisoxazol- |
| | 3-yl)nicotinamide |
| CPD- | 2-((2-(2-(4-(6-((6-acetyl-8-cyclopentyl-5-methyl-7-oxo-7,8-dihydropyrido[2,3- |
| 235 | d]pyrimidin-2-yl)amino)pyridin-3-yl)piperazin-1-yl)acetyl)-2-azaspiro[3.4]octan-6- |
| | yl)amino)-N-(6-(dimethylamino)pyridazin-3-yl)-6-(methylamino)nicotinamide |
| CPD- | 2-((1-(2-(4-(6-((6-acetyl-8-cyclopentyl-5-methyl-7-oxo-7,8-dihydropyrido[2,3- |
| 236 | d]pyrimidin-2-yl)amino)pyridin-3-yl)piperazin-1-yl)acetyl)piperidin-4-yl)amino)- |
| | N-(2-cyclopropyl-1-methyl-1H-imidazol-4-yl)-6-((methyl-d₃)amino)nicotinamide |
| CPD- | 2-((6-(2-(4-(6-((6-acetyl-8-cyclopentyl-5-methyl-7-oxo-7,8-dihydropyrido[2,3- |
| 237 | d]pyrimidin-2-yl)amino)pyridin-3-yl)piperazin-1-yl)acetamido)spiro[3.3]heptan-2- |
| | yl)amino)-N-(2-cyclopropyl-1-methyl-1H-imidazol-4-yl)-6-((methyl- |
| | d₃)amino)nicotinamide |
| CPD- | 2-((6-(2-(4-(6-((6-acetyl-8-cyclopentyl-5-methyl-7-oxo-7,8-dihydropyrido[2,3- |
| 238 | d]pyrimidin-2-yl)amino)pyridin-3-yl)piperazin-1-yl)acetamido)spiro[3.3]heptan-2- |
| | yl)amino)-N-(1-isopropyl-2-methyl-1H-imidazol-4-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 239 | d]pyrimidin-2-yl)amino)pyridin-3-yl)piperazin-1-yl)acetamido)spiro[3.3]heptan-2- |
| | yl)amino)-N-(6-cyclopropyl-5-fluoropyridin-2-yl)-6-((methyl- |
| | d₃)amino)nicotinamide |
| CPD- | 2-((3-(2-(4-(6-((6-acetyl-8-cyclopentyl-5-methyl-7-oxo-7,8-dihydropyrido[2,3- |
| 240 | d]pyrimidin-2-yl)amino)pyridin-3-yl)piperazin-1-yl)acetamido)cyclobutyl)amino)- |
| | N-(6-cyclopropylpyridin-2-yl)-6-(methylamino)nicotinamide |
| CPD- | 2-((6-(2-(4-(6-((6-acetyl-8-cyclopentyl-5-methyl-7-oxo-7,8-dihydropyrido[2,3- |
| 241 | d]pyrimidin-2-yl)amino)pyridin-3-yl)piperazin-1-yl)acetamido)spiro[3.3]heptan-2- |
| | yl)amino)-N-(6-(dimethylamino)pyridazin-3-yl)-6-((methyl-d₃)amino)nicotinamide |
| CPD- | 2-((3-(2-(4-(6-((6-acetyl-8-cyclopentyl-5-methyl-7-oxo-7,8-dihydropyrido[2,3- |
| 242 | d]pyrimidin-2-yl)amino)pyridin-3-yl)piperazin-1- |
| | yl)propanamido)cyclobutyl)amino)-N-(6-(dimethylamino)pyridazin-3-yl)-6- |
| | (methylamino)nicotinamide |
| CPD- | 2-((3-(2-(4-(6-((6-acetyl-8-cyclopentyl-5-methyl-7-oxo-7,8-dihydropyrido[2,3- |
| 243 | d]pyrimidin-2-yl)amino)pyridin-3-yl)piperazin-1-yl)-2- |
| | methylpropanamido)cyclobutyl)amino)-N-(6-(dimethylamino)pyridazin-3-yl)-6- |
| | (methylamino)nicotinamide |
| CPD- | 2-((6-(2-(4-(6-((6-acetyl-8-cyclopentyl-5-methyl-7-oxo-7,8-dihydropyrido[2,3- |
| 244 | d]pyrimidin-2-yl)amino)pyridin-3-yl)piperazin-1-yl)acetamido)spiro[3.3]heptan-2- |
| | yl)amino)-N-(1,5-dimethyl-1H-pyrazol-3-yl)-6-((methyl-d₃)amino)nicotinamide |
| CPD- | 2-((3-(2-(4-(6-((6-acetyl-8-cyclopentyl-5-methyl-7-oxo-7,8-dihydropyrido[2,3- |
| 245 | d]pyrimidin-2-yl)amino)pyridin-3-yl)piperazin-1-yl)acetamido)cyclobutyl)amino)- |
| | N-(6-(dimethylamino)pyridazin-3-yl)-6-((methyl-d₃)amino)nicotinamide |
| CPD- | 2-((6-(2-(4-(6-((6-acetyl-8-cyclopentyl-5-methyl-7-oxo-7,8-dihydropyrido[2,3- |
| 246 | d]pyrimidin-2-yl)amino)pyridin-3-yl)piperazin-1-yl)acetamido)spiro[3.3]heptan-2- |
| | yl)amino)-6-(methylamino)-N-(5-methyloxazol-2-yl)nicotinamide |
| CPD- | 2-((6-(2-(4-(6-((6-acetyl-8-cyclopentyl-5-methyl-7-oxo-7,8-dihydropyrido[2,3- |
| 247 | d]pyrimidin-2-yl)amino)pyridin-3-yl)piperazin-1-yl)acetamido)spiro[3.3]heptan-2- |
| | yl)amino)-N-(4,5-dimethyloxazol-2-yl)-6-(methylamino)nicotinamide |
| CPD- | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-2,2- |
| 248 | difluoropropanoyl)azetidin-3-yl)amino)-N-(6-(dimethylamino)pyridazin-3-yl)-6- |
| | ((methyl-d₃)amino)nicotinamide |
| CPD- | 2-((2-(3-(4-(6-((6-acetyl-8-cyclopentyl-5-methyl-7-oxo-7,8-dihydropyrido[2,3- |
| 249 | d]pyrimidin-2-yl)amino)pyridin-3-yl)piperazin-1-yl)-2,2-difluoropropanoyl)-2- |
| | azaspiro[3.3]heptan-6-yl)amino)-N-(6-(dimethylamino)pyridazin-3-yl)-6-((methyl- |
| | d₃)amino)nicotinamide |
| CPD- | 2-((1-(2-(4-(6-((6-acetyl-8-cyclopentyl-5-methyl-7-oxo-7,8-dihydropyrido[2,3- |
| 250 | d]pyrimidin-2-yl)amino)pyridin-3-yl)piperazin-1-yl)acetyl)piperidin-4-yl)amino)- |
| | N-(6-(dimethylamino)pyridazin-3-yl)-6-((methyl-d₃)amino)nicotinamide |
| CPD- | 6-((5-(2-(4-(6-((6-acetyl-8-cyclopentyl-5-methyl-7-oxo-7,8-dihydropyrido[2,3- |
| 251 | d]pyrimidin-2-yl)amino)pyridin-3-yl)piperazin-1-yl)acetamido)pentyl)amino)-N-(6- |
| | (dimethylamino)pyridazin-3-yl)-1H-pyrrolo[2,3-b]pyridine-5-carboxamide |
| CPD- | 2-((1-(2-(4-(6-((6-acetyl-8-cyclopentyl-5-methyl-7-oxo-7,8-dihydropyrido[2,3- |
| 252 | d]pyrimidin-2-yl)amino)pyridin-3-yl)piperazin-1-yl)acetyl)piperidin-4-yl)amino)- |
| | N-(6-cyclopropylpyridin-2-yl)-6-((methyl-d₃)amino)nicotinamide |
| CPD- | 2-((6-(2-(4-(6-((6-acetyl-8-cyclopentyl-5-methyl-7-oxo-7,8-dihydropyrido[2,3- |
| 253 | d]pyrimidin-2-yl)amino)pyridin-3-yl)piperazin-1-yl)acetamido)spiro[3.3]heptan-2- |
| | yl)amino)-N-(1,5-dimethyl-1H-pyrazol-3-yl)-6-(isopropylamino)nicotinamide |
| CPD- | 2-((3-((2-(4-(6-((6-acetyl-8-cyclopentyl-5-methyl-7-oxo-7,8-dihydropyrido[2,3- |
| 254 | d]pyrimidin-2-yl)amino)pyridin-3-yl)piperazin-1- |
| | yl)acetamido)methyl)cyclobutyl)amino)-N-(2-cyclopropyl-1-methyl-1H-imidazol- |
| | 4-yl)-6-(methylamino)nicotinamide |
| CPD- | 2-((3-(2-(2-(4-(6-((6-acetyl-8-cyclopentyl-5-methyl-7-oxo-7,8-dihydropyrido[2,3- |
| 255 | d]pyrimidin-2-yl)amino)pyridin-3-yl)piperazin-1- |
| | yl)acetamido)ethyl)cyclobutyl)amino)-4-chloro-N-(6-cyclopropyl-5-methylpyridin- |
| | 2-yl)benzamide |
| CPD- | 4-(6-((6-acetyl-8-cyclopentyl-5-methyl-7-oxo-7,8-dihydropyrido[2,3-d]pyrimidin- |
| 256 | 2-yl)amino)pyridin-3-yl)-N-(2-(3-((5-chloro-2-((6-cyclopropyl-5-methylpyridin-2- |
| | yl)carbamoyl)phenyl)amino)cyclobutyl)ethyl)piperazine-1-carboxamide |
| CPD- | 2-((3-(2-(2-(4-(6-((6-acetyl-8-cyclopentyl-5-methyl-7-oxo-7,8-dihydropyrido[2,3- |
| 257 | d]pyrimidin-2-yl)amino)pyridin-3-yl)piperazin-1- |
| | yl)acetamido)ethyl)cyclobutyl)amino)-4-chloro-N-(6-(dimethylamino)pyridazin-3- |
| | yl)benzamide |
| CPD- | 2-((3-(2-(2-(4-(6-((6-acetyl-8-cyclopentyl-5-methyl-7-oxo-7,8-dihydropyrido[2,3- |
| 258 | d]pyrimidin-2-yl)amino)pyridin-3-yl)piperazin-1- |
| | yl)acetamido)ethyl)cyclobutyl)amino)-N-(6-(dimethylamino)pyridazin-3-yl)-4- |
| | fluorobenzamide |
| CPD- | 2-((6-(2-(4-(6-((6-acetyl-8-cyclopentyl-5-methyl-7-oxo-7,8-dihydropyrido[2,3- |
| 259 | d]pyrimidin-2-yl)amino)pyridin-3-yl)piperazin-1-yl)acetamido)spiro[3.3]heptan-2- |
| | yl)amino)-4-chloro-N-(5-methylisoxazol-3-yl)benzamide |
| CPD- | 2-((6-(2-(4-(6-((6-acetyl-8-cyclopentyl-5-methyl-7-oxo-7,8-dihydropyrido[2,3- |
| 260 | d]pyrimidin-2-yl)amino)pyridin-3-yl)piperazin-1-yl)acetamido)spiro[3.3]heptan-2- |
| | yl)amino)-4-fluoro-N-(5-methylisoxazol-3-yl)benzamide |
| CPD- | 2-((3-((2-(4-(6-((6-acetyl-8-cyclopentyl-5-methyl-7-oxo-7,8-dihydropyrido[2,3- |
| 261 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-4- |
| | (trifluoromethyl)benzamide |
| CPD- | 2-((3-((2-(4-(6-((6-acetyl-8-cyclopentyl-5-methyl-7-oxo-7,8-dihydropyrido[2,3- |
| 262 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-5- |
| | (trifluoromethyl)benzamide |
| CPD- | 2-((3-((2-(4-(6-((6-acetyl-8-cyclopentyl-5-methyl-7-oxo-7,8-dihydropyrido[2,3- |
| 263 | d]pyrimidin-2-yl)amino)pyridin-3-yl)piperazin- 1- |
| | yl)acetamido)methyl)cyclobutyl)amino)-N-(1,5-dimethyl-1H-pyrazol-3-yl)-5- |
| | fluorobenzamide |
| CPD- | 2-((3-((2-(4-(6-((6-acetyl-8-cyclopentyl-5-methyl-7-oxo-7,8-dihydropyrido[2,3- |
| 264 | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-4- |
| | fluorobenzamide |

TABLE 4B
Additional representative heterobifunctional compounds.

| Cpd. No. | Chemical Name |
|---|---|
| CPD-265 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-3- |
| | fluorobenzamide |
| CPD-266 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(1,5-dimethyl-1H-pyrazol-3-yl)-3- |
| | (trifluoromethyl)benzamide |
| CPD-267 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)-2,2-dimethylpiperazin-1-yl)propanoyl)-2- |
| | azaspiro[3.3]heptan-6-yl)amino)-N-(6-(dimethylamino)pyridazin-3-yl)-6- |
| | (methylamino)nicotinamide |
| CPD-268 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[3.3]heptan-6-yl)amino)-N-(6-(dimethylamino)pyridazin-3-yl)-6- |
| | (methylamino)nicotinamide |
| CPD-269 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)azetidin-3- |
| | yl)amino)-N-(6-(dimethylamino)pyridazin-3-yl)-6-(methylamino)nicotinamide |
| CPD-270 | 2-((1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)piperidin-4-yl)amino)- |
| | 6-((methyl-d₃)amino)-N-(6-methylpyridin-2-yl)nicotinamide |
| CPD-271 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclopentyl)amino)-N-(6-(dimethylamino)pyridazin-3-yl)-6- |
| | (methylamino)nicotinamide |
| CPD-272 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(6-(dimethylamino)pyridazin-3-yl)-6- |
| | (isopropylamino)nicotinamide |
| CPD-273 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)pyrrolidin-3- |
| | yl)amino)-N-(2-cyclopropyl-1-methyl-1H-imidazol-4-yl)-6-((methyl- |
| | d3)amino)nicotinamide |
| CPD-274 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)pyrrolidin-3- |
| | yl)amino)-N-(2-cyclopropyl-1-methyl-1H-imidazol-4-yl)-6- |
| | (isopropylamino)nicotinamide |
| CPD-275 | 2-(((trans-4-(2-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)acetamido)cyclohexyl)methyl)amino)-N-(2-cyclopropyl-1-methyl-1H- |
| | imidazol-4-yl)-6-(isopropylamino)nicotinamide |
| CPD-276 | 2-(((trans-4-(2-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)acetamido)cyclohexyl)methyl)amino)-N-(2-cyclopropyl-1-methyl-1H- |
| | imidazol-4-yl)-6-((methyl-d₃)amino)nicotinamide |
| CPD-277 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(2-cyclopropyl-1-methyl-1H-imidazol- |
| | 4-yl)-6-(isopropylamino)nicotinamide |
| CPD-278 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(2-cyclopropyl-1-methyl-1H-imidazol- |
| | 4-yl)-6-((methyl-d₃)amino)nicotinamide |
| CPD-279 | 2-((1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)piperidin-4-yl)amino)- |
| | N-(2-cyclopropyl-1-methyl-1H-imidazol-4-yl)-6-(isopropylamino)nicotinamide |
| CPD-280 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclobutyl)amino)-N-(6-(dimethylamino)pyridazin-3-yl)-6- |
| | (isopropylamino)nicotinamide |
| CPD-281 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-6-(methylamino)-N-(5-methylisoxazol-3- |
| | yl)nicotinamide |
| CPD-282 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-6-(isopropylamino)-N-(5- |
| | methylisoxazol-3-yl)nicotinamide |
| CPD-283 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-6-((methyl-d₃)amino)-N-(5- |
| | methylisoxazol-3-yl)nicotinamide |
| CPD-284 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclopentyl)amino)-N-(2-cyclopropyl-1-methyl-1H-imidazol-4-yl)- |
| | 6-(isopropylamino)nicotinamide |
| CPD-285 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclopentyl)amino)-N-(2-cyclopropyl-1-methyl-1H-imidazol-4-yl)- |
| | 6-((methyl-d₃)amino)nicotinamide |
| CPD-286 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-4-methyl-N-(5-methylisoxazol-3-yl)benzamide |
| CPD-287 | 4-(6-((6-acetyl-8-cyclopentyl-5-methyl-7-oxo-7,8-dihydropyrido[2,3-d]pyrimidin- |
| | 2-yl)amino)pyridin-3-yl)-N-(3-((3-((2-cyclopropyl-1-methyl-1H-imidazol-4- |
| | yl)carbamoyl)-6-((methyl-d₃)amino)pyridin-2-yl)amino)cyclobutyl)piperazine-1- |
| | carboxamide |
| CPD-288 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclobutyl)amino)-N-(2-cyclopropyl-1-methyl-1H-imidazol-4-yl)-6- |
| | (isopropylamino)nicotinamide |
| CPD-289 | 4-(6-((6-acetyl-8-cyclopentyl-5-methyl-7-oxo-7,8-dihydropyrido[2,3-d]pyrimidin- |
| | 2-yl)amino)pyridin-3-yl)-N-(3-((3-((2-cyclopropyl-1-methyl-1H-imidazol-4- |
| | yl)carbamoyl)-6-(isopropylamino)pyridin-2-yl)amino)cyclobutyl)piperazine-1- |
| | carboxamide |
| CPD-290 | 2-((1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)piperidin-4-yl)amino)- |
| | 6-(isopropylamino)-N-(5-methylisoxazol-3-yl)nicotinamide |
| CPD-291 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6- |
| | (dimethylamino)nicotinamide |
| CPD-292 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6- |
| | isopropoxynicotinamide |
| CPD-293 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6- |
| | (ethylamino)nicotinamide |
| CPD-294 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclopentyl)amino)-6-((methyl-d3)amino)-N-(5-methylisoxazol-3- |
| | yl)nicotinamide |
| CPD-295 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclopentyl)amino)-6-(isopropylamino)-N-(5-methylisoxazol-3- |
| | yl)nicotinamide |
| CPD-296 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopentyl-1-methyl-1H-imidazol-4-yl)-6- |
| | (methylamino)nicotinamide |
| CPD-297 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(1-cyclobutyl-5-methyl-1H-pyrazol-3-yl)-6- |
| | (methylamino)nicotinamide |
| CPD-298 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6- |
| | (propylamino)nicotinamide |
| CPD-299 | 2-((1-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)glycyl)piperidin-4-yl)amino)- |
| | N-(6-(dimethylamino)pyridazin-3-yl)-6-(methylamino)nicotinamide |
| CPD-300 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(6-(dimethylamino)pyridazin-3-yl)-6-(methylamino)nicotinamide |
| CPD-301 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)piperidin-4- |
| | yl)amino)-N-(6-(dimethylamino)pyridazin-3-yl)-6-(isopropylamino)nicotinamide |
| CPD-302 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)-2- |
| | azaspiro[3.3]heptan-6-yl)amino)-N-(1,5-dimethyl-1H-pyrazol-3-yl)-6- |
| | (isopropylamino)nicotinamide |
| CPD-303 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)azetidin-3- |
| | yl)amino)-N-(6-(dimethylamino)pyridazin-3-yl)-6-(isopropylamino)nicotinamide |
| CPD-304 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)azetidin-3- |
| | yl)amino)-N-(2-cyclopropyl-1-methyl-1H-imidazol-4-yl)-6- |
| | (isopropylamino)nicotinamide |
| CPD-305 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)-2- |
| | azaspiro[3.3]heptan-6-yl)amino)-N-(6-(dimethylamino)pyridazin-3-yl)-6- |
| | (isopropylamino)nicotinamide |
| CPD-306 | 2-((2-(1-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)cyclopropane-1- |
| | carbonyl)-2-azaspiro[3.3]heptan-6-yl)amino)-N-(6-(dimethylamino)pyridazin-3- |
| | yl)-6-(isopropylamino)nicotinamide |
| CPD-307 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)cyclobutyl)amino)-N-(2-cyclopropyl-1-methyl-1H-imidazol-4-yl)-6- |
| | ((methyl-d₃)amino)nicotinamide |
| CPD-308 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-ethyl-1-methyl-1H-imidazol-4-yl)-6- |
| | (methylamino)nicotinamide |
| CPD-309 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(1-methyl-2-propyl-1H-imidazol-4-yl)-6- |
| | (methylamino)nicotinamide |
| CPD-310 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclobutyl-1-methyl-1H-imidazol-4-yl)-6- |
| | (methylamino)nicotinamide |
| CPD-311 | 3-((3-((6-(dimethylamino)pyridazin-3-yl)carbamoyl)-6-(methylamino)pyridin-2- |
| | yl)amino)cyclobutyl 4-(6-((6-acetyl-8-cyclopentyl-5-methyl-7-oxo-7,8- |
| | dihydropyrido[2,3-d]pyrimidin-2-yl)amino)pyridin-3-yl)piperazine-1-carboxylate |
| CPD-312 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(1-ethyl-5-methyl-1H-pyrazol-3-yl)-6-(methylamino)nicotinamide |
| CPD-313 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)piperidin-4- |
| | yl)amino)-N-(2-cyclopropyl-1-methyl-1H-imidazol-4-yl)-6- |
| | (isopropylamino)nicotinamide |
| CPD-314 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(5-methyl-1-propyl-1H-pyrazol-3-yl)-6- |
| | (methylamino)nicotinamide |
| CPD-315 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(1-cyclopropyl-5-methyl-1H-pyrazol-3-yl)-6- |
| | (methylamino)nicotinamide |
| CPD-316 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(6-(dimethylamino)pyridazin-3-yl)-6- |
| | (isopropylamino)nicotinamide |
| CPD-317 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)oxy)-N-(2-cyclopropyl-1-methyl-1H-imidazol-4-yl)-6- |
| | (isopropylamino)nicotinamide |
| CPD-318 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6- |
| | isobutylnicotinamide |
| CPD-319 | 2-((3-(3-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)pyrrolidin-1- |
| | yl)cyclobutyl)amino)-N-(6-(dimethylamino)pyridazin-3-yl)-6- |
| | (methylamino)nicotinamide |
| CPD-320 | 2-((2-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)-2- |
| | azaspiro[3.3]heptan-6-yl)amino)-N-(2-cyclopropyl-1-methyl-1H-imidazol-4-yl)- |
| | 6-(methylamino)nicotinamide |
| CPD-321 | 2-((2-(7-(6-((6-acetyl-8-cyclopentyl-5-methyl-7-oxo-7,8-dihydropyrido[2,3- |
| | d]pyrimidin-2-yl)amino)pyridin-3-yl)-7-azaspiro[3.5]nonane-2-carbonyl)-2- |
| | azaspiro[3.3]heptan-6-yl)amino)-N-(2-cyclopropyl-1-methyl-1H-imidazol-4-yl)- |
| | 6-(methylamino)nicotinamide |
| CPD-322 | 2-((2-(1-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)cyclopropane-1- |
| | carbonyl)-2-azaspiro[3.3]heptan-6-yl)amino)-N-(2-cyclopropyl-1-methyl-1H- |
| | imidazol-4-yl)-6-(isopropylamino)nicotinamide |
| CPD-323 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)-2- |
| | azaspiro[3.3]heptan-6-yl)amino)-N-(2-cyclopropyl-1-methyl-1H-imidazol-4-yl)- |
| | 6-(isopropylamino)nicotinamide |
| CPD-324 | 2-((2-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)-2- |
| | azaspiro[3.3]heptan-6-yl)amino)-N-(6-(dimethylamino)pyridazin-3-yl)-6- |
| | (methylamino)nicotinamide |
| CPD-325 | 2-((2-(7-(6-((6-acetyl-8-cyclopentyl-5-methyl-7-oxo-7,8-dihydropyrido[2,3- |
| | d]pyrimidin-2-yl)amino)pyridin-3-yl)-7-azaspiro[3.5]nonane-2-carbonyl)-2- |
| | azaspiro[3.3]heptan-6-yl)amino)-N-(6-(dimethylamino)pyridazin-3-yl)-6- |
| | (methylamino)nicotinamide |
| CPD-326 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)cyclobutyl)amino)-N-(6-(dimethylamino)pyridazin-3-yl)-6- |
| | (isopropylamino)nicotinamide |
| CPD-327 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)cyclobutyl)amino)-N-(2-cyclopropyl-1-methyl-1H-imidazol-4- |
| | yl)-6-(isopropylamino)nicotinamide |
| CPD-328 | 2-((1-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)piperidin- |
| | 4-yl)amino)-N-(6-(dimethylamino)pyridazin-3-yl)-6-(methylamino)nicotinamide |
| CPD-329 | 2-((1-(7-(6-((6-acetyl-8-cyclopentyl-5-methyl-7-oxo-7,8-dihydropyrido[2,3- |
| | d]pyrimidin-2-yl)amino)pyridin-3-yl)-7-azaspiro[3.5]nonane-2- |
| | carbonyl)piperidin-4-yl)amino)-N-(6-(dimethylamino)pyridazin-3-yl)-6- |
| | (methylamino)nicotinamide |
| CPD-330 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-(1-hydroxyethyl)-1-methyl-1H-imidazol-4-yl)-6- |
| | (methylamino)nicotinamide |
| CPD-331 | 2-((3-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[3.5]nonane-2- |
| | carbonyl)cyclobutyl)amino)-N-(6-(dimethylamino)pyridazin-3-yl)-6- |
| | (methylamino)nicotinamide |
| CPD-332 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)azetidine-1- |
| | carbonyl)cyclobutyl)amino)-N-(6-(dimethylamino)pyridazin-3-yl)-6- |
| | (methylamino)nicotinamide |
| CPD-333 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclobutyl)amino)-N-(6-(dimethylamino)pyridazin-3-yl)-6- |
| | (methylamino)nicotinamide |
| CPD-334 | 2-((3-(4-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)piperidine-1- |
| | carbonyl)cyclobutyl)amino)-N-(6-(dimethylamino)pyridazin-3-yl)-6- |
| | (methylamino)nicotinamide |
| CPD-335 | 2-((3-(4-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)-1H-1,2,3-triazol-1- |
| | yl)cyclobutyl)amino)-N-(6-(dimethylamino)pyridazin-3-yl)-6- |
| | (methylamino)nicotinamide |
| CPD-336 | 2-(((3-((3-((4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)methyl)pyrrolidin-1- |
| | yl)methyl)cyclobutyl)amino)-N-(6-(dimethylamino)pyridazin-3-yl)-6- |
| | (methylamino)nicotinamide |
| CPD-337 | 2-((3-(9-(6-((6-acetyl-8-cyclopentyl-5-methyl-7-oxo-7,8-dihydropyrido[2,3- |
| | d]pyrimidin-2-yl)amino)pyridin-3-yl)-3,9-diazaspiro[5.5]undecane-3- |
| | carbonyl)cyclobutyl)amino)-N-(6-(dimethylamino)pyridazin-3-yl)-6- |
| | (methylamino)nicotinamide |
| CPD-338 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)-2- |
| | azaspiro[3.3]heptan-6-yl)amino)-N-(2-cyclopropyl-1-methyl-1H-imidazol-4-yl)- |
| | 6-(isopropylamino)nicotinamide |
| CPD-339 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)-2- |
| | azaspiro[3.3]heptan-6-yl)amino)-N-(2-cyclopropyl-1-methyl-1H-imidazol-4-yl)- |
| | 6-((methyl-d₃)amino)nicotinamide |
| CPD-340 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-(2-hydroxypropan-2-yl)-1-methyl-1H-imidazol-4-yl)-6- |
| | (methylamino)nicotinamide |
| CPD-341 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-342 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-343 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)cyclobutyl)amino)-N-(6-(dimethylamino)pyridazin-3-yl)-6- |
| | ((methyl-d₃)amino)nicotinamide |
| CPD-344 | 2-(((1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)-3-fluoroazetidin-3- |
| | yl)methyl)amino)-N-(6-(dimethylamino)pyridazin-3-yl)-6- |
| | (methylamino)nicotinamide |
| CPD-345 | 2-(((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)bicyclo[1.1.1]pentan-1-yl)methyl)amino)-N-(6- |
| | (dimethylamino)pyridazin-3-yl)-6-(methylamino)nicotinamide |
| CPD-346 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)bicyclo[1.1.1 ]pentan-1-yl)amino)-N-(6-(dimethylamino)pyridazin- |
| | 3-yl)-6-(methylamino)nicotinamide |
| CPD-347 | 2-((2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.5]nonan-7- |
| | yl)amino)-N-(2-cyclopropyl-1-methyl-1H-imidazol-4-yl)-6- |
| | (methylamino)nicotinamide |
| CPD-348 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)cyclobutane-1-carbonyl)-2- |
| | azaspiro[3.3]heptan-6-yl)amino)-N-(6-(dimethylamino)pyridazin-3-yl)-6- |
| | (methylamino)nicotinamide |
| CPD-349 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6-(1- |
| | hydroxyethyl)nicotinamide |
| CPD-350 | 2-((4-(9-(6-((6-acetyl-8-cyclopentyl-5-methyl-7-oxo-7,8-dihydropyrido[2,3- |
| | d]pyrimidin-2-yl)amino)pyridin-3-yl)-3,9-diazaspiro[5.5]undecane-3- |
| | carbonyl)cyclohexyl)amino)-N-(6-(dimethylamino)pyridazin-3-yl)-6- |
| | (methylamino)nicotinamide |
| CPD-351 | 2-((4-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[3.5]nonane-2- |
| | carbonyl)cyclohexyl)amino)-N-(6-(dimethylamino)pyridazin-3-yl)-6- |
| | (methylamino)nicotinamide |
| CPD-352 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(2-cyclopropyl-1-methyl-1H-imidazol-4-yl)-6-(2-hydroxypropan- |
| | 2-yl)nicotinamide |
| CPD-353 | 2-((3-(1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)ethyl)cyclobutyl)amino)-N-(1,5-dimethyl-1H-pyrazol-3-yl)-6- |
| | ((methyl-d₃)amino)nicotinamide |
| CPD-354 | 2-(((2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)-5-oxa-2- |
| | azaspiro[3.4]octan-7-yl)methyl)amino)-N-(6-(dimethylamino)pyridazin-3-yl)-6- |
| | (methylamino)nicotinamide |
| CPD-355 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)azetidin-3- |
| | yl)amino)-N-(6-(dimethylamino)pyridazin-3-yl)-6-(isopropylamino)nicotinamide |
| CPD-356 | 2-((1-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)glycyl)azetidin-3-yl)amino)- |
| | N-(6-(dimethylamino)pyridazin-3-yl)-6-(isopropylamino)nicotinamide |
| CPD-357 | 2-((1-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)glycyl)piperidin-4-yl)amino)- |
| | N-(6-(dimethylamino)pyridazin-3-yl)-6-(isopropylamino)nicotinamide |
| CPD-358 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(6-(dimethylamino)pyridazin-3-yl)-6-(isopropylamino)nicotinamide |
| CPD-359 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)piperidin-4- |
| | yl)amino)-N-(6-(dimethylamino)pyridazin-3-yl)-6-(isopropylamino)nicotinamide |
| CPD-360 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)piperidin-4- |
| | yl)amino)-N-(6-(dimethylamino)pyridazin-3-yl)-6-(isopropylamino)nicotinamide |
| CPD-361 | 2-((3-(1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)ethyl)cyclobutyl)amino)-6-((methyl-d₃)amino)-N-(5- |
| | methylisoxazol-3-yl)nicotinamide |
| CPD-362 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)azetidine-1- |
| | carbonyl)spiro[3.3]heptan-2-yl)amino)-N-(6-(dimethylamino)pyridazin-3-yl)-6- |
| | (methylamino)nicotinamide |
| CPD-363 | 2-((6-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[3.5]nonane-2- |
| | carbonyl)spiro[3.3]heptan-2-yl)amino)-N-(6-(dimethylamino)pyridazin-3-yl)-6- |
| | (methylamino)nicotinamide |
| CPD-364 | 2-((2-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)azetidine-3-carbonyl)-2- |
| | azaspiro[3.3]heptan-6-yl)amino)-N-(6-(dimethylamino)pyridazin-3-yl)-6- |
| | (methylamino)nicotinamide |
| CPD-365 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)butanamido)cyclobutyl)amino)-N-(6-(dimethylamino)pyridazin-3-yl)-6- |
| | (isopropylamino)nicotinamide |
| CPD-366 | 2-((6-(1-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)cyclopropane-1- |
| | carboxamido)spiro[3.3]heptan-2-yl)amino)-N-(2-cyclopropyl-1-methyl-1H- |
| | imidazol-4-yl)-6-(isopropylamino)nicotinamide |
| CPD-367 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(6-(dimethylamino)pyridazin-3-yl)-6- |
| | (isopropylamino)nicotinamide |
| CPD-368 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)-2- |
| | azaspiro[3.3]heptan-6-yl)amino)-N-(6-(dimethylamino)pyridazin-3-yl)-6- |
| | (isopropylamino)nicotinamide |
| CPD-369 | 2-((1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetyl)piperidin-4-yl)amino)- |
| | N-(6-(dimethylamino)pyridazin-3-yl)-6-(isopropylamino)nicotinamide |
| CPD-370 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)pyrrolidin-3- |
| | yl)amino)-N-(6-(dimethylamino)pyridazin-3-yl)-6-(isopropylamino)nicotinamide |
| CPD-371 | 2-((6-(1-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)cyclopropane-1- |
| | carboxamido)spiro[3.3]heptan-2-yl)amino)-6-(isopropylamino)-N-(5- |
| | methylisoxazol-3-yl)nicotinamide |
| CPD-372 | 2-((1-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)glycyl)pyrrolidin-3- |
| | yl)amino)-N-(6-(dimethylamino)pyridazin-3-yl)-6-(isopropylamino)nicotinamide |
| CPD-373 | 2-((2-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.4]octan-2-yl)acetyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-374 | 2-((6-((7-(6-((6-acetyl-8-cyclopentyl-5-methyl-7-oxo-7,8-dihydropyrido[2,3- |
| | d]pyrimidin-2-yl)amino)pyridin-3-yl)-7-azaspiro[3.5]nonan-2- |
| | yl)carbamoyl)spiro[3.3]heptan-2-yl)amino)-N-(6-(dimethylamino)pyridazin-3-yl)- |
| | 6-(methylamino)nicotinamide |
| CPD-375 | 2-((3-((7-(6-((6-acetyl-8-cyclopentyl-5-methyl-7-oxo-7,8-dihydropyrido[2,3- |
| | d]pyrimidin-2-yl)amino)pyridin-3-yl)-7-azaspiro[3.5]nonan-2- |
| | yl)carbamoyl)cyclobutyl)amino)-N-(6-(dimethylamino)pyridazin-3-yl)-6- |
| | (methylamino)nicotinamide |
| CPD-376 | 2-((6-(3-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)azetidine-1- |
| | carbonyl)spiro[3.3]heptan-2-yl)amino)-N-(6-(dimethylamino)pyridazin-3-yl)-6- |
| | (methylamino)nicotinamide |
| CPD-377 | 2-((3-(1-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)ethyl)cyclobutyl)amino)-6-(isopropylamino)-N-(5-methylisoxazol- |
| | 3-yl)nicotinamide |
| CPD-378 | 2-((6-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.4]octane-2- |
| | carbonyl)spiro[3.3]heptan-2-yl)amino)-N-(6-(dimethylamino)pyridazin-3-yl)-6- |
| | ((methyl-d₃)amino)nicotinamide |
| CPD-379 | 2-((6-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonane-2- |
| | carbonyl)spiro[3.3]heptan-2-yl)amino)-N-(6-(dimethylamino)pyridazin-3-yl)-6- |
| | ((methyl-d₃)amino)nicotinamide |
| CPD-380 | 2-((6-(8-(6-((6-acetyl-8-cyclopentyl-5-methyl-7-oxo-7,8-dihydropyrido[2,3- |
| | d]pyrimidin-2-yl)amino)pyridin-3-yl)-2,8-diazaspiro[4.5]decane-2- |
| | carbonyl)spiro[3.3]heptan-2-yl)amino)-N-(6-(dimethylamino)pyridazin-3-yl)-6- |
| | ((methyl-d₃)amino)nicotinamide |
| CPD-381 | 2-((3-(8-(6-((6-acetyl-8-cyclopentyl-5-methyl-7-oxo-7,8-dihydropyrido[2,3- |
| | d]pyrimidin-2-yl)amino)pyridin-3-yl)-2,8-diazaspiro[4.5]decane-2- |
| | carbonyl)cyclobutyl)amino)-N-(6-(dimethylamino)pyridazin-3-yl)-6- |
| | (methylamino)nicotinamide |
| CPD-382 | 2-((3-(2-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4- |
| | yl)amino)acetamido)cyclobutyl)amino)-N-(6-(dimethylamino)pyridazin-3-yl)-6- |
| | (isopropylamino)nicotinamide |
| CPD-383 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)-6- |
| | azaspiro[2.5]octan-1-yl)amino)-N-(6-(dimethylamino)pyridazin-3-yl)-6-((methyl- |
| | d₃)amino)nicotinamide |
| CPD-384 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-385 | 2-((4-(8-(6-((6-acetyl-8-cyclopentyl-5-methyl-7-oxo-7,8-dihydropyrido[2,3- |
| | d]pyrimidin-2-yl)amino)pyridin-3-yl)-2,8-diazaspiro[4.5]decane-2- |
| | carbonyl)cyclohexyl)amino)-N-(6-(dimethylamino)pyridazin-3-yl)-6- |
| | (methylamino)nicotinamide |
| CPD-386 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-1-yl)acetyl)piperidin-4- |
| | yl)amino)-N-(6-(dimethylamino)pyridazin-3-yl)-6-(isopropylamino)nicotinamide |
| CPD-387 | 2-((3-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)propan-2- |
| | yl)cyclobutyl)amino)-N-(1,5-dimethyl-1H-pyrazol-3-yl)-6- |
| | (isopropylamino)nicotinamide |
| CPD-388 | 2-((3-((3-(6-((6-acetyl-8-cyclopentyl-5-methyl-7-oxo-7,8-dihydropyrido[2,3- |
| | d]pyrimidin-2-yl)amino)pyridin-3-yl)-3-azaspiro[5.5]undecan-9- |
| | yl)carbamoyl)cyclobutyl)amino)-N-(6-(dimethylamino)pyridazin-3-yl)-6- |
| | (methylamino)nicotinamide |
| CPD-389 | 2-((1-((4-(6-((6-acetyl-8-cyclopentyl-5-methyl-7-oxo-7,8-dihydropyrido[2,3- |
| | d]pyrimidin-2-yl)amino)pyridin-3-yl)cyclohexyl)glycyl)piperidin-4-yl)amino)-N- |
| | (6-(dimethylamino)pyridazin-3-yl)-6-(isopropylamino)nicotinamide |
| CPD-390 | 2-((1-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)azetidine-3- |
| | carbonyl)piperidin-4-yl)amino)-N-(6-(dimethylamino)pyridazin-3-yl)-6- |
| | (isopropylamino)nicotinamide |
| CPD-391 | 2-((2-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)azetidine-3-carbonyl)-2- |
| | azaspiro[3.3]heptan-6-yl)amino)-N-(6-(dimethylamino)pyridazin-3-yl)-6- |
| | (isopropylamino)nicotinamide |
| CPD-392 | 2-((1-(2-(1-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-4-yl)acetyl)piperidin-4- |
| | yl)amino)-N-(6-(dimethylamino)pyridazin-3-yl)-6-(isopropylamino)nicotinamide |
| CPD-393 | 2-((6-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)azetidine-3- |
| | carboxamido)spiro[3.3]heptan-2-yl)amino)-N-(6-(dimethylamino)pyridazin-3-yl)- |
| | 6-(isopropylamino)nicotinamide |
| CPD-394 | 2-((1-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carbonyl)azetidin-3-yl)amino)-N-(6-(dimethylamino)pyridazin-3-yl)-6- |
| | (isopropylamino)nicotinamide |
| CPD-395 | 2-((1-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carbonyl)pyrrolidin-3-yl)amino)-N-(6-(dimethylamino)pyridazin-3-yl)-6- |
| | (isopropylamino)nicotinamide |
| CPD-396 | 2-((1-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carbonyl)piperidin-4-yl)amino)-N-(6-(dimethylamino)pyridazin-3-yl)-6- |
| | (isopropylamino)nicotinamide |
| CPD-397 | 2-((6-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carboxamido)spiro[3.3]heptan-2-yl)amino)-N-(6-(dimethylamino)pyridazin-3-yl)- |
| | 6-(isopropylamino)nicotinamide |
| CPD-398 | 2-((2-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3-carbonyl)-2- |
| | azaspiro[3.3]heptan-6-yl)amino)-N-(6-(dimethylamino)pyridazin-3-yl)-6- |
| | (isopropylamino)nicotinamide |
| CPD-399 | 2-((-4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(6-(dimethylamino)pyridazin-3-yl)-6- |
| | (isopropylamino)nicotinamide |
| CPD-400 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)spiro[3.3]heptan-2-yl)amino)-N-(6-(dimethylamino)pyridazin-3-yl)-6- |
| | (isopropylamino)nicotinamide |
| CPD-401 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)azetidine-1- |
| | carbonyl)spiro[3.3]heptan-2-yl)amino)-N-(6-(dimethylamino)pyridazin-3-yl)-6- |
| | (isopropylamino)nicotinamide |
| CPD-402 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclobutyl)amino)-N-(6-(dimethylamino)pyridazin-3-yl)-6- |
| | (isopropylamino)nicotinamide |
| CPD-403 | 2-(((1R,3S)-3-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclopentyl)amino)-N-(6-(dimethylamino)pyridazin-3- |
| | yl)-6-(isopropylamino)nicotinamide |
| CPD-404 | 2-((2-(3-(4-(6-((6-acetyl-8-cyclopentyl-5-methyl-7-oxo-7,8-dihydropyrido[2,3- |
| | d]pyrimidin-2-yl)(methyl)amino)pyridin-3-yl)piperazin-1-yl)propanoyl)-2- |
| | azaspiro[3.3]heptan-6-yl)amino)-N-(1,5-dimethyl-1H-pyrazol-3-yl)-6- |
| | (isopropylamino)nicotinamide |
| CPD-405 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)spiro[3.3]heptan-2-yl)amino)-N-(2-cyclopropyl-1-methyl-1H- |
| | imidazol-4-yl)-6-((methyl-d₃)amino)nicotinamide |
| CPD-406 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)spiro[3.3]heptan-2-yl)amino)-N-(6-(dimethylamino)pyridazin-3- |
| | yl)-6-(isopropylamino)nicotinamide |
| CPD-407 | 2-((1-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)piperidin- |
| | 4-yl)amino)-N-(6-(dimethylamino)pyridazin-3-yl)-6- |
| | (isopropylamino)nicotinamide |
| CPD-408 | 2-((1-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)piperidin- |
| | 4-yl)amino)-N-(1,5-dimethyl-1H-pyrazol-3-yl)-6-(isopropylamino)nicotinamide |
| CPD-409 | 2-((1-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)piperidin- |
| | 4-yl)amino)-N-(2-cyclopropyl-1-methyl-1H-imidazol-4-yl)-6- |
| | (isopropylamino)nicotinamide |
| CPD-410 | 2-((3-(2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)propan-2- |
| | yl)cyclobutyl)amino)-6-(isopropylamino)-N-(5-methylisoxazol-3-yl)nicotinamide |
| CPD-411 | 2-((2-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)-2- |
| | azaspiro[3.3]heptan-6-yl)amino)-N-(6-(dimethylamino)pyridazin-3-yl)-6- |
| | (isopropylamino)nicotinamide |
| CPD-412 | 2-((2-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)-2- |
| | azaspiro[3.3]heptan-6-yl)amino)-N-(2-cyclopropyl-1-methyl-1H-imidazol-4-yl)- |
| | 6-(isopropylamino)nicotinamide |
| CPD-413 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(3-((3-((6-(dimethylamino)pyridazin-3- |
| | yl)carbamoyl)-6-(isopropylamino)pyridin-2-yl)amino)cyclobutyl)-6- |
| | azaspiro[2.5]octane-1-carboxamide |
| CPD-414 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(6-((3-((6-(dimethylamino)pyridazin-3- |
| | yl)carbamoyl)-6-(isopropylamino)pyridin-2-yl)amino)spiro[3.3]heptan-2-yl)-6- |
| | azaspiro[2.5]octane-1-carboxamide |
| CPD-415 | 2-((2-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)-2- |
| | azaspiro[3.3]heptan-6-yl)amino)-N-(1,5-dimethyl-1H-pyrazol-3-yl)-6- |
| | (isopropylamino)nicotinamide |
| CPD-416 | 2-((2-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.4]octan-6- |
| | yl)amino)-N-(6-(dimethylamino)pyridazin-3-yl)-6-(methylamino)nicotinamide |
| CPD-417 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(3-((3-((6-(dimethylamino)pyridazin-3- |
| | yl)carbamoyl)-6-(isopropylamino)pyridin-2-yl)amino)cyclopentyl)-6- |
| | azaspiro[2.5]octane-1-carboxamide |
| CPD-418 | 2-((1-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)acetyl)piperidin-4-yl)amino)-N-(6-(dimethylamino)pyridazin-3-yl)-6- |
| | (isopropylamino)nicotinamide |
| CPD-419 | 2-((1-(6-(6-((6-acetyl-8-cyclopentyl-5-methyl-7-oxo-7,8-dihydropyrido[2,3- |
| | d]pyrimidin-2-yl)amino)pyridin-3-yl)-6-azaspiro[2.5]octane-1-carbonyl)piperidin- |
| | 4-yl)amino)-6-(isopropylamino)-N-(5-methylisoxazol-3-yl)nicotinamide |
| CPD-420 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(2-cyclopropyl-1-methyl-1H-imidazol-4-yl)-6- |
| | (isopropylamino)nicotinamide |
| CPD-421 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(1,5-dimethyl-1H-pyrazol-3-yl)-6-(isopropylamino)nicotinamide |
| CPD-422 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[3.3]heptan-6-yl)amino)-N-(6-(dimethylamino)pyridazin-3-yl)-6- |
| | (isopropylamino)nicotinamide |
| CPD-423 | 2-((2-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2-yl)acetyl)-2- |
| | azaspiro[3.3]heptan-6-yl)amino)-N-(6-(dimethylamino)pyridazin-3-yl)-6- |
| | (isopropylamino)nicotinamide |
| CPD-424 | 2-((1-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)acetyl)pyrrolidin-3-yl)amino)-N-(6-(dimethylamino)pyridazin-3-yl)-6- |
| | (isopropylamino)nicotinamide |
| CPD-425 | 2-((2-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)-2- |
| | azaspiro[3.3]heptan-6-yl)amino)-N-(6-(dimethylamino)pyridazin-3-yl)-6- |
| | (isopropylamino)nicotinamide |
| CPD-426 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)pyrrolidin-3- |
| | yl)amino)-N-(6-(dimethylamino)pyridazin-3-yl)-6-(isopropylamino)nicotinamide |
| CPD-427 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)azetidin-3- |
| | yl)amino)-N-(6-(dimethylamino)pyridazin-3-yl)-6-(isopropylamino)nicotinamide |
| CPD-428 | 2-((3-((8-(6-((6-acetyl-8-cyclopentyl-5-methyl-7-oxo-7,8-dihydropyrido[2,3- |
| | d]pyrimidin-2-yl)amino)pyridin-3-yl)-8-azaspiro[4.5]decan-2- |
| | yl)carbamoyl)cyclobutyl)amino)-N-(6-(dimethylamino)pyridazin-3-yl)-6- |
| | (methylamino)nicotinamide |
| CPD-429 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)acetamido)methyl)bicyclo[1.1.1]pentan-1-yl)amino)-N-(6- |
| | (dimethylamino)pyridazin-3-yl)-6-(isopropylamino)nicotinamide |
| CPD-430 | 2-((3-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-N- |
| | methylacetamido)cyclopentyl)amino)-N-(6-(dimethylamino)pyridazin-3-yl)-6- |
| | (methylamino)nicotinamide |
| CPD-431 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)butanoyl)piperidin-4- |
| | yl)amino)-N-(2-cyclopropyl-1-methyl-1H-imidazol-4-yl)-6- |
| | (isopropylamino)nicotinamide |
| CPD-432 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)propanoyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-433 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(1,5-dimethyl-1H-pyrazol-3-yl)-6- |
| | (isopropylamino)nicotinamide |
| CPD-434 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(1,5-dimethyl-1H-pyrazol-3-yl)-6-((methyl- |
| | d3)amino)nicotinamide |
| CPD-435 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(2-cyclopropyl-1-methyl-1H-imidazol-4-yl)- |
| | 6-((methyl-d₃)amino)nicotinamide |
| CPD-436 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(2-cyclopropyl-1-methyl-1H-imidazol-4-yl)- |
| | 6-(isopropylamino)nicotinamide |
| CPD-437 | 7-(6-((6-acetyl-8-cyclopentyl-5-methyl-7-oxo-7,8-dihydropyrido[2,3-d]pyrimidin- |
| | 2-yl)amino)pyridin-3-yl)-N-(6-((3-((6-(dimethylamino)pyridazin-3- |
| | yl)carbamoyl)-6-(isopropylamino)pyridin-2-yl)amino)spiro[3.3]heptan-2-yl)-7- |
| | azaspiro[3.5]nonane-2-carboxamide |
| CPD-438 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)spiro[3.3]heptan-2-yl)amino)-N-(6-(dimethylamino)pyridazin-3- |
| | yl)-6-((methyl-d₃)amino)nicotinamide |
| CPD-439 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(6-((3-((6-(dimethylamino)pyridazin-3- |
| | yl)carbamoyl)-6-((methyl-d₃)amino)pyridin-2-yl)amino)spiro[3.3]heptan-2-yl)-6- |
| | azaspiro[2.5]octane-1-carboxamide |
| CPD-440 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(5,6-dihydro-4H-pyrrolo[1,2-b]pyrazol-2-yl)-6- |
| | (isopropylamino)nicotinamide |
| CPD-441 | 2-((1-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetyl)piperidin-4-yl)amino)-N-(6-(dimethylamino)pyridazin-3-yl)-6-((methyl- |
| | d₃)amino)nicotinamide |
| CPD-442 | 2-((1-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetyl)azetidin-3-yl)amino)-N-(6-(dimethylamino)pyridazin-3-yl)-6-((methyl- |
| | d₃)amino)nicotinamide |
| CPD-443 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(2-cyclopropyl-1-methyl-1H-imidazol-4-yl)- |
| | 6-((methyl-d₃)amino)nicotinamide |
| CPD-444 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(2-cyclopropyl-1-methyl-1H-imidazol-4-yl)- |
| | 6-(isopropylamino)nicotinamide |
| CPD-445 | 2-((3-((2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetamido)methyl)cyclobutyl)amino)-N-(6-(dimethylamino)pyridazin-3-yl)-6- |
| | ((methyl-d₃)amino)nicotinamide |
| CPD-446 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(1,5-dimethyl-1H-pyrazol-3-yl)-6- |
| | (isopropylamino)nicotinamide |
| CPD-447 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(1,5-dimethyl-1H-pyrazol-3-yl)-6-((methyl- |
| | d₃)amino)nicotinamide |
| CPD-448 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(6-(dimethylamino)pyridazin-3-yl)-6- |
| | (isopropylamino)nicotinamide |
| CPD-449 | 2-((6-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetamido)spiro[3.3]heptan-2-yl)amino)-N-(6-(dimethylamino)pyridazin-3-yl)- |
| | 6-((methyl-d₃)amino)nicotinamide |
| CPD-450 | 2-((3-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetamido)cyclobutyl)amino)-N-(6-(dimethylamino)pyridazin-3-yl)-6- |
| | ((methyl-d₃)amino)nicotinamide |
| CPD-451 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(6-((3-((1,5-dimethyl-1H-pyrazol-3-yl)carbamoyl)-6- |
| | (isopropylamino)pyridin-2-yl)amino)spiro[3.3]heptan-2-yl)-6- |
| | azaspiro[2.5]octane-1-carboxamide |
| CPD-452 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(1-cyclopropyl-5-methyl-1H-pyrazol-3-yl)-6- |
| | (isopropylamino)nicotinamide |
| CPD-453 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-6-(isopropylamino)-N-(6-methoxypyridin-2-yl)nicotinamide |
| CPD-454 | 2-((1-(3-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)propanoyl)piperidin-4-yl)amino)-N-(6-(dimethylamino)pyridazin-3-yl)-6- |
| | (isopropylamino)nicotinamide |
| CPD-455 | 2-((2-(3-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2-yl)propanoyl)- |
| | 2-azaspiro[3.3]heptan-6-yl)amino)-N-(6-(dimethylamino)pyridazin-3-yl)-6- |
| | (isopropylamino)nicotinamide |
| CPD-456 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)propanoyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-457 | 6-(6-((6-acetyl-8-cyclopentyl-5-methyl-7-oxo-7,8-dihydropyrido[2,3-d]pyrimidin- |
| | 2-yl)amino)pyridin-3-yl)-N-(6-((3-((2-cyclopropyl-1-methyl-1H-imidazol-4- |
| | yl)carbamoyl)-6-(isopropylamino)pyridin-2-yl)amino)spiro[3.3]heptan-2-yl)-6- |
| | azaspiro[2.5]octane-1-carboxamide |
| CPD-458 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)-2,2-dimethylpiperazin-1-yl)propanoyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-459 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)-2,2-dimethylpiperazin-1-yl)propanoyl)-2- |
| | azaspiro[3.3]heptan-6-yl)amino)-N-(6-(dimethylamino)pyridazin-3-yl)-6- |
| | (isopropylamino)nicotinamide |
| CPD-460 | 2-((3-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carboxamido)cyclobutyl)amino)-N-(6-(dimethylamino)pyridazin-3-yl)-6- |
| | (isopropylamino)nicotinamide |
| CPD-461 | 2-((3-((1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carboxamido)methyl)cyclobutyl)amino)-N-(6-(dimethylamino)pyridazin-3-yl)-6- |
| | (isopropylamino)nicotinamide |
| CPD-462 | 2-((4-(1-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)pyrrolidine-3- |
| | carboxamido)cyclohexyl)amino)-N-(6-(dimethylamino)pyridazin-3-yl)-6- |
| | (isopropylamino)nicotinamide |
| CPD-463 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)piperidin-4- |
| | yl)amino)-N-(6-(dimethylamino)pyridazin-3-yl)-6-((methyl- |
| | d₃)amino)nicotinamide |
| CPD-464 | 2-((2-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-465 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)piperidin-4- |
| | yl)amino)-N-(1,5-dimethyl-1H-pyrazol-3-yl)-6-(isopropylamino)nicotinamide |
| CPD-466 | 2-((1-(N-(1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-N-methylglycyl)piperidin-4- |
| | yl)amino)-N-(2-cyclopropyl-1-methyl-1H-imidazol-4-yl)-6- |
| | (isopropylamino)nicotinamide |
| CPD-467 | 2-((6-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)acetamido)spiro[3.3]heptan-2-yl)amino)-N-(6-(dimethylamino)pyridazin-3-yl)- |
| | 6-(isopropylamino)nicotinamide |
| CPD-468 | 2-((3-((2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)acetamido)methyl)cyclobutyl)amino)-N-(6-(dimethylamino)pyridazin-3-yl)-6- |
| | (isopropylamino)nicotinamide |
| CPD-469 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)piperidin-4-yl)amino)-N-(6-(dimethylamino)pyridazin-3-yl)-6- |
| | (isopropylamino)nicotinamide |
| CPD-470 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carboxamido)spiro[3.3]heptan-2-yl)amino)-N-(6-(dimethylamino)pyridazin-3-yl)- |
| | 6-(isopropylamino)nicotinamide |
| CPD-471 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclobutyl)amino)-N-(6-(dimethylamino)pyridazin-3-yl)-6-((methyl- |
| | d3)amino)nicotinamide |
| CPD-472 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(6-(dimethylamino)pyridazin-3-yl)- |
| | 6-(isopropylamino)nicotinamide |
| CPD-473 | 2-((4-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)cyclohexyl)amino)-N-(6-(dimethylamino)pyridazin-3-yl)-6- |
| | (isopropylamino)nicotinamide |
| CPD-474 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(6-(dimethylamino)pyridazin-3-yl)-6-((methyl- |
| | d₃)amino)nicotinamide |
| CPD-475 | 2-((3-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)cyclobutyl)amino)-N-(6-(dimethylamino)pyridazin-3-yl)-6- |
| | (isopropylamino)nicotinamide |
| CPD-476 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-6-((methyl-d₃)amino)nicotinamide |
| CPD-477 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(1,5-dimethyl-1H-pyrazol-3-yl)-6-((methyl- |
| | d₃)amino)nicotinamide |
| CPD-478 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(1,5-dimethyl-1H-pyrazol-3-yl)-6- |
| | (isopropylamino)nicotinamide |
| CPD-479 | 2-((3-((2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)methyl)bicyclo[1.1.1]pentan-1-yl)amino)-N-(6- |
| | (dimethylamino)pyridazin-3-yl)-6-(isopropylamino)nicotinamide |
| CPD-480 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(2-cyclopropyl-1-methyl-1H-imidazol-4-yl)-6- |
| | ((methyl-d₃)amino)nicotinamide |
| CPD-481 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(2-cyclopropyl-1-methyl-1H-imidazol-4-yl)-6- |
| | (isopropylamino)nicotinamide |
| CPD-482 | 2-((1-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)piperidin-4-yl)amino)-N-(6-(dimethylamino)pyridazin-3-yl)-6-((methyl- |
| | d₃)amino)nicotinamide |
| CPD-483 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carboxamido)cyclohexyl)amino)-N-(6-(dimethylamino)pyridazin-3-yl)-6- |
| | (isopropylamino)nicotinamide |
| CPD-484 | 2-((1-(N-(2-(6-((6-acetyl-8-cyclopentyl-5-methyl-7-oxo-7,8-dihydropyrido[2,3- |
| | d]pyrimidin-2-yl)amino)pyridin-3-yl)-2-azaspiro[3.3]heptan-6-yl)-N- |
| | methylglycyl)piperidin-4-yl)amino)-N-(6-(dimethylamino)pyridazin-3-yl)-6- |
| | (isopropylamino)nicotinamide |
| CPD-485 | 2-((3-((2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetamido)methyl)bicyclo[1.1.1]pentan-1-yl)amino)-N-(6- |
| | (dimethylamino)pyridazin-3-yl)-6-(isopropylamino)nicotinamide |
| CPD-486 | 2-((6-((1-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-4- |
| | yl)carbamoyl)spiro[3.3]heptan-2-yl)amino)-N-(6-(dimethylamino)pyridazin-3-yl)- |
| | 6-(isopropylamino)nicotinamide |
| CPD-487 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(6-(dimethylamino)pyridazin-3-yl)-6- |
| | (isopropylamino)nicotinamide |
| CPD-488 | 2-((2-(2-(4-(1-(6-((6-acetyl-8-cyclopentyl-5-methyl-7-oxo-7,8-dihydropyrido[2,3- |
| | d]pyrimidin-2-yl)amino)pyridin-3-yl)ethyl)piperazin-1-yl)acetyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-489 | 2-((2-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2-yl)acetyl)-2- |
| | azaspiro[3.3]heptan-6-yl)amino)-N-(6-(dimethylamino)pyridazin-3-yl)-6- |
| | ((methyl-d₃)amino)nicotinamide |
| CPD-490 | 2-((1-(2-(6-(6-((6-acetyl-8-cyclopentyl-5-methyl-7-oxo-7,8-dihydropyrido[2,3- |
| | d]pyrimidin-2-yl)amino)pyridin-3-yl)-2,6-diazaspiro[3.3]heptan-2- |
| | yl)acetyl)piperidin-4-yl)amino)-N-(6-(dimethylamino)pyridazin-3-yl)-6-((methyl- |
| | d₃)amino)nicotinamide |
| CPD-491 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)-2- |
| | oxoethyl)piperidin-4-yl)amino)-N-(6-(dimethylamino)pyridazin-3-yl)-6-((methyl- |
| | d₃)amino)nicotinamide |
| CPD-492 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(6-(dimethylamino)pyridazin-3-yl)-6- |
| | ethylnicotinamide |
| CPD-493 | 2-((3-((2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)methyl)cyclobutyl)amino)-N-(6-(dimethylamino)pyridazin-3-yl)-6- |
| | (isopropylamino)nicotinamide |
| CPD-494 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carboxamido)cyclobutyl)amino)-N-(6-(dimethylamino)pyridazin-3-yl)-6- |
| | ((methyl-d₃)amino)nicotinamide |
| CPD-495 | 2-((4-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazine-1- |
| | carbonyl)cyclohexyl)amino)-N-(6-(dimethylamino)pyridazin-3-yl)-6-((methyl- |
| | d₃)amino)nicotinamide |
| CPD-496 | 2-((3-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2- |
| | yl)acetamido)bicyclo[1.1.1]pentan-1-yl)amino)-N-(6-(dimethylamino)pyridazin- |
| | 3-yl)-6-(isopropylamino)nicotinamide |
| CPD-497 | 2-((2-(2-(6-((6-((6-acetyl-8-cyclopentyl-5-methyl-7-oxo-7,8-dihydropyrido[2,3- |
| | d]pyrimidin-2-yl)amino)pyridin-3-yl)methyl)-2,6-diazaspiro[3.3]heptan-2-yl)-2- |
| | oxoethyl)-2-azaspiro[3.3]heptan-6-yl)amino)-N-(6-(dimethylamino)pyridazin-3- |
| | yl)-6-(isopropylamino)nicotinamide |
| CPD-498 | 2-((1-(2-(6-((6-((6-acetyl-8-cyclopentyl-5-methyl-7-oxo-7,8-dihydropyrido[2,3- |
| | d]pyrimidin-2-yl)amino)pyridin-3-yl)methyl)-2,6-diazaspiro[3.3]heptan-2-yl)-2- |
| | oxoethyl)piperidin-4-yl)amino)-N-(6-(dimethylamino)pyridazin-3-yl)-6- |
| | (isopropylamino)nicotinamide |
| CPD-499 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-6-isopropylnicotinamide |
| CPD-500 | 2-((trans-4-(3-(4-(6-((6-acetyl-8-cyclopentyl-5-ethyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-6-(isopropylamino)-N-(6- |
| | methoxypyridazin-3-yl)nicotinamide |
| CPD-501 | 2-((3-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)cyclopentyl)amino)-N-(6-(dimethylamino)pyridazin-3-yl)-6- |
| | (isopropylamino)nicotinamide |
| CPD-502 | 2-((3-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)bicyclo[1.1.1]pentan-1-yl)amino)-N-(6-(dimethylamino)pyridazin- |
| | 3-yl)-6-(isopropylamino)nicotinamide |
| CPD-503 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)propanamido)bicyclo[1.1.1]pentan-1-yl)amino)-N-(6- |
| | (dimethylamino)pyridazin-3-yl)-6-(isopropylamino)nicotinamide |
| CPD-504 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)butanamido)bicyclo[1.1.1]pentan-1-yl)amino)-N-(6-(dimethylamino)pyridazin- |
| | 3-yl)-6-(isopropylamino)nicotinamide |
| CPD-505 | 2-((3-(2-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4- |
| | yl)(methyl)amino)acetamido)bicyclo[1.1.1]pentan-1-yl)amino)-N-(6- |
| | (dimethylamino)pyridazin-3-yl)-6-(isopropylamino)nicotinamide |
| CPD-506 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1- |
| | yl)ethyl)carbamoyl)bicyclo[1.1.1]pentan-1-yl)amino)-N-(6- |
| | (dimethylamino)pyridazin-3-yl)-6-(isopropylamino)nicotinamide |
| CPD-507 | 2-((2-(2-(7-((6-((6-acetyl-8-cyclopentyl-5-methyl-7-oxo-7,8-dihydropyrido[2,3- |
| | d]pyrimidin-2-yl)amino)pyridin-3-yl)methyl)-2,7-diazaspiro[4.4]nonan-2-yl)-2- |
| | oxoethyl)-2-azaspiro[3.3]heptan-6-yl)amino)-N-(6-(dimethylamino)pyridazin-3- |
| | yl)-6-((methyl-d₃)amino)nicotinamide |
| CPD-508 | 2-((2-(2-(6-((6-((6-acetyl-8-cyclopentyl-5-methyl-7-oxo-7,8-dihydropyrido[2,3- |
| | d]pyrimidin-2-yl)amino)pyridin-3-yl)methyl)-2,6-diazaspiro[3.3]heptan-2- |
| | yl)acetyl)-2-azaspiro[3.3]heptan-6-yl)amino)-N-(6-(dimethylamino)pyridazin-3- |
| | yl)-6-(isopropylamino)nicotinamide |
| CPD-509 | 2-((1-(2-(6-((6-((6-acetyl-8-cyclopentyl-5-methyl-7-oxo-7,8-dihydropyrido[2,3- |
| | d]pyrimidin-2-yl)amino)pyridin-3-yl)methyl)-2,6-diazaspiro[3.3]heptan-2- |
| | yl)acetyl)piperidin-4-yl)amino)-N-(6-(dimethylamino)pyridazin-3-yl)-6- |
| | (isopropylamino)nicotinamide |
| CPD-510 | 2-((3-(3-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)pyrrolidine-1- |
| | carbonyl)cyclobutyl)amino)-N-(6-(dimethylamino)pyridazin-3-yl)-6- |
| | (isopropylamino)nicotinamide |
| CPD-511 | 2-((3-(3-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)pyrrolidine-1- |
| | carbonyl)cyclopentyl)amino)-N-(6-(dimethylamino)pyridazin-3-yl)-6- |
| | (isopropylamino)nicotinamide |
| CPD-512 | 2-((4-(3-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)pyrrolidine-1- |
| | carbonyl)cyclohexyl)amino)-N-(6-(dimethylamino)pyridazin-3-yl)-6- |
| | (isopropylamino)nicotinamide |
| CPD-513 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)-2-oxoethyl)azetidin- |
| | 3-yl)amino)-N-(6-(dimethylamino)pyridazin-3-yl)-6-(methylamino)nicotinamide |
| CPD-514 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-6-(isopropylamino)-N-(5-methyl-1-(trifluoromethyl)-1H-pyrazol-3- |
| | yl)nicotinamide |
| CPD-515 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[3.4]octan-6-yl)amino)-N-(6-(dimethylamino)pyridazin-3-yl)-6- |
| | (isopropylamino)nicotinamide |
| CPD-516 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-4- |
| | methylcyclohexyl)amino)-N-(6-(dimethylamino)pyridazin-3-yl)-6- |
| | (isopropylamino)nicotinamide |
| CPD-517 | 2-((1-(2-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)(methyl)amino)-2- |
| | oxoethyl)azetidin-3-yl)amino)-N-(6-(dimethylamino)pyridazin-3-yl)-6- |
| | (isopropylamino)nicotinamide |
| CPD-518 | 2-((2-(2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidin-1-yl)-2-oxoethyl)- |
| | 2-azaspiro[3.3]heptan-6-yl)amino)-N-(6-(dimethylamino)pyridazin-3-yl)-6- |
| | (isopropylamino)nicotinamide |
| CPD-519 | 2-((2-(2-(7-((6-((6-acetyl-8-cyclopentyl-5-methyl-7-oxo-7,8-dihydropyrido[2,3- |
| | d]pyrimidin-2-yl)amino)pyridin-3-yl)methyl)-2,7-diazaspiro[4.4]nonan-2- |
| | yl)acetyl)-2-azaspiro[3.3]heptan-6-yl)amino)-N-(6-(dimethylamino)pyridazin-3- |
| | yl)-6-((methyl-d₃)amino)nicotinamide |
| CPD-520 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(5,6-dihydro-4H-pyrrolo[1,2- |
| | b]pyrazol-2-yl)-6-(isopropylamino)nicotinamide |
| CPD-521 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(5,6-dihydro-4H-pyrrolo[1,2- |
| | b]pyrazol-2-yl)-6-((methyl-d₃)amino)nicotinamide |
| CPD-522 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-6-(isopropylamino)-N-(6- |
| | methoxypyridin-2-yl)nicotinamide |
| CPD-523 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-methoxypyridin-2-yl)-6- |
| | ((methyl-d₃)amino)nicotinamide |
| CPD-524 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(5,6-dihydro-4H-pyrrolo[1,2-b]pyrazol-2-yl)- |
| | 6-((methyl-d₃)amino)nicotinamide |
| CPD-525 | 2-((3-((2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-N- |
| | methylacetamido)methyl)bicyclo[1.1.1]pentan-1-yl)amino)-N-(6- |
| | (dimethylamino)pyridazin-3-yl)-6-(isopropylamino)nicotinamide |
| CPD-526 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-4-ethylbenzamide |
| CPD-527 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-4-(isopropylamino)benzamide |
| CPD-528 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-4-isopropylbenzamide |
| CPD-529 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(5,6-dihydro-4H-pyrrolo[1,2-b]pyrazol-2-yl)- |
| | 6-(isopropylamino)nicotinamide |
| CPD-530 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(5,6-dihydro-4H-pyrrolo[1,2-b]pyrazol-2-yl)-6- |
| | (isopropylamino)nicotinamide |
| CPD-531 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(6-methoxypyridin-2-yl)-6-((methyl- |
| | d₃)amino)nicotinamide |
| CPD-532 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-6-(isopropylamino)-N-(6-methoxypyridin-2- |
| | yl)nicotinamide |
| CPD-533 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-6-(isopropylamino)-N-(6-methoxypyridin-2-yl)nicotinamide |
| CPD-534 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(6-(dimethylamino)pyridazin-3-yl)- |
| | 6-(isopropylamino)nicotinamide |
| CPD-535 | 2-((4-(3-(4-(6-((8-cyclopentyl-7-oxo-7,8-dihydropyrido[2,3-d]pyrimidin-2- |
| | yl)amino)pyridin-3-yl)piperazin-1-yl)pyrrolidine-1-carbonyl)cyclohexyl)amino)- |
| | N-(6-(dimethylamino)pyridazin-3-yl)-6-(isopropylamino)nicotinamide |
| CPD-536 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-4- |
| | methoxycyclohexyl)amino)-N-(6-(dimethylamino)pyridazin-3-yl)-6- |
| | (isopropylamino)nicotinamide |
| CPD-537 | 2-((3-(2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidin-1-yl)-2- |
| | oxoethyl)bicyclo[1.1.1]pentan-1-yl)amino)-N-(6-(dimethylamino)pyridazin-3-yl)- |
| | 6-(isopropylamino)nicotinamide |
| CPD-538 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)-2-oxoethyl)-2- |
| | azaspiro[3.3]heptan-6-yl)amino)-N-(6-(dimethylamino)pyridazin-3-yl)-6- |
| | (isopropylamino)nicotinamide |
| CPD-539 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-6- |
| | azaspiro[3.4]octan-2-yl)amino)-N-(6-(dimethylamino)pyridazin-3-yl)-6- |
| | (isopropylamino)nicotinamide |
| CPD-540 | trans-6-acetyl-8-cyclopentyl-2-((5-(4-(1-(4-((3-(5-(dimethylamino)-1H- |
| | benzo[d]imidazol-2-yl)-6-(isopropylamino)pyridin-2-yl)amino)cyclohexane-1- |
| | carbonyl)pyrrolidin-3-yl)piperazin-1-yl)pyridin-2-yl)amino)-5-methylpyrido[2,3- |
| | d]pyrimidin-7(8H)-one |
| CPD-541 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)-2- |
| | oxoethyl)piperidin-4-yl)amino)-N-(6-(dimethylamino)pyridazin-3-yl)-6- |
| | (isopropylamino)nicotinamide |
| CPD-542 | (S)-2-((3-((1-(1-(6-((6-acetyl-8-cyclopentyl-5-methyl-7-oxo-7,8- |
| | dihydropyrido[2,3-d]pyrimidin-2-yl)amino)pyridin-3-yl)piperidin-4- |
| | yl)pyrrolidine-2-carboxamido)methyl)cyclobutyl)amino)-N-(6- |
| | (dimethylamino)pyridazin-3-yl)-6-(isopropylamino)nicotinamide |
| CPD-543 | (S)-2-((6-(1-(1-(6-((6-acetyl-8-cyclopentyl-5-methyl-7-oxo-7,8- |
| | dihydropyrido[2,3-d]pyrimidin-2-yl)amino)pyridin-3-yl)piperidin-4- |
| | yl)pyrrolidine-2-carboxamido)spiro[3.3]heptan-2-yl)amino)-N-(6- |
| | (dimethylamino)pyridazin-3-yl)-6-(isopropylamino)nicotinamide |
| CPD-544 | 2-((2-((1-(6-((6-acetyl-8-cyclopentyl-5-methyl-7-oxo-7,8-dihydropyrido[2,3- |
| | d]pyrimidin-2-yl)amino)pyridin-3-yl)piperidin-4-yl)-L-prolyl)-2- |
| | azaspiro[3.3]heptan-6-yl)amino)-N-(6-(dimethylamino)pyridazin-3-yl)-6- |
| | (isopropylamino)nicotinamide |
| CPD-545 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-2- |
| | azaspiro[3.4]octan-6-yl)amino)-N-(6-(dimethylamino)pyridazin-3-yl)-6- |
| | (isopropylamino)nicotinamide |
| CPD-546 | trans-6-acetyl-8-cyclopentyl-2-((5-(4-(1-(4-((3-(5-(dimethylamino)-3H- |
| | imidazo[4,5-b]pyridin-2-yl)-6-(isopropylamino)pyridin-2-yl)amino)cyclohexane- |
| | 1-carbonyl)pyrrolidin-3-yl)piperazin-1-yl)pyridin-2-yl)amino)-5- |
| | methylpyrido[2,3-d]pyrimidin-7(8H)-one |
| CPD-547 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(1,5-dimethyl-1H-pyrazol-3-yl)-6- |
| | (isopropylamino)nicotinamide |
| CPD-548 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(2-cyclopropyl-1-methyl-1H-imidazol-4-yl)-6- |
| | (isopropylamino)nicotinamide |
| CPD-549 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(5,6-dihydro-4H-pyrrolo[1,2-b]pyrazol-2-yl)- |
| | 6-(isopropylamino)nicotinamide |
| CPD-550 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)propanoyl)-2- |
| | azaspiro[4.4]nonan-7-yl)amino)-N-(6-(dimethylamino)pyridazin-3-yl)-6- |
| | (isopropylamino)nicotinamide |
| CPD-551 | 2-((2-(2-(4-(1-(6-((6-acetyl-8-cyclopentyl-5-methyl-7-oxo-7,8-dihydropyrido[2,3- |
| | d]pyrimidin-2-yl)amino)pyridin-3-yl)ethyl)piperazin-1-yl)acetyl)-2- |
| | azaspiro[4.4]nonan-7-yl)amino)-N-(6-(dimethylamino)pyridazin-3-yl)-6- |
| | (isopropylamino)nicotinamide |
| CPD-552 | trans-6-acetyl-8-cyclopentyl-2-((5-(4-(1-(trans-4-((3-(6-(dimethylamino)-3H- |
| | imidazo[4,5-c]pyridin-2-yl)-6-(isopropylamino)pyridin-2-yl)amino)cyclohexane- |
| | 1-carbonyl)pyrrolidin-3-yl)piperazin-1-yl)pyridin-2-yl)amino)-5- |
| | methylpyrido[2,3-d]pyrimidin-7(8H)-one |
| CPD-553 | N-(6-(dimethylamino)pyridazin-3-yl)-2-((2-(2-(4-((6-((4-(2-ethyl-1-oxo-1,2,3,4- |
| | tetrahydroisoquinolin-6-yl)-5-fluoropyrimidin-2-yl)amino)pyridin-3- |
| | yl)methyl)piperazin-1-yl)acetyl)-2-azaspiro[3.3]heptan-6-yl)amino)-6- |
| | (isopropylamino)nicotinamide |
| CPD-554 | N-(6-(dimethylamino)pyridazin-3-yl)-2-((6-(2-(4-((6-((4-(2-ethyl-1-oxo-1,2,3,4- |
| | tetrahydroisoquinolin-6-yl)-5-fluoropyrimidin-2-yl)amino)pyridin-3- |
| | yl)methyl)piperazin-1-yl)acetamido)spiro[3.3]heptan-2-yl)amino)-6- |
| | (isopropylamino)nicotinamide |
| CPD-555 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-2- |
| | azaspiro[3.5]nonan-7-yl)amino)-N-(6-(dimethylamino)pyridazin-3-yl)-6- |
| | (isopropylamino)nicotinamide |
| CPD-556 | 2-((6-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)spiro[3.4]octan-2-yl)amino)-N-(6-(dimethylamino)pyridazin-3-yl)-6- |
| | (isopropylamino)nicotinamide |
| CPD-557 | N-(6-(dimethylamino)pyridazin-3-yl)-6-(isopropylamino)-2-((2-(2-(4-((6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)methyl)piperazin-1-yl)acetyl)-2- |
| | azaspiro[3.3]heptan-6-yl)amino)nicotinamide |
| CPD-558 | N-(6-(dimethylamino)pyridazin-3-yl)-6-(isopropylamino)-2-((6-(2-(4-((6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)methyl)piperazin-1- |
| | yl)acetamido)spiro[3.3]heptan-2-yl)amino)nicotinamide |
| CPD-559 | 2-((trans-4-(3-(4-(6-((8-cyclopentyl-6-(difluoromethyl)-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-6-(isopropylamino)nicotinamide |
| CPD-560 | 2-((trans-4-(3-(4-(6-((6-acetyl-8-cyclopentyl-7-oxo-7,8-dihydropyrido[2,3- |
| | d]pyrimidin-2-yl)amino)pyridin-3-yl)piperazin-1-yl)pyrrolidine-1- |
| | carbonyl)cyclohexyl)amino)-N-(6-(dimethylamino)pyridazin-3-yl)-6- |
| | (isopropylamino)nicotinamide |
| CPD-561 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-2- |
| | azaspiro[4.4]nonan-7-yl)amino)-N-(6-(dimethylamino)pyridazin-3-yl)-6- |
| | (isopropylamino)nicotinamide |
| CPD-562 | 7-cyclopentyl-2-((5-((4-(2-((6-((3-((6-(dimethylamino)pyridazin-3-yl)carbamoyl)- |
| | 6-(isopropylamino)pyridin-2-yl)amino)spiro[3.3]heptan-2-yl)amino)-2- |
| | oxoethyl)piperazin-1-yl)methyl)pyridin-2-yl)amino)-N,N-dimethyl-7H- |
| | pyrrolo[2,3-d]pyrimidine-6-carboxamide |
| CPD-563 | 7-cyclopentyl-2-((5-((4-(2-(6-((3-((6-(dimethylamino)pyridazin-3-yl)carbamoyl)- |
| | 6-(isopropylamino)pyridin-2-yl)amino)-2-azaspiro[3.3]heptan-2-yl)-2- |
| | oxoethyl)piperazin-1-yl)methyl)pyridin-2-yl)amino)-N,N-dimethyl-7H- |
| | pyrrolo[2,3-d]pyrimidine-6-carboxamide |
| CPD-564 | 2-((1-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)piperidin-4- |
| | yl)amino)-N-(6,7-dihydro-5H-pyrrolo[1,2-a]imidazol-2-yl)-6- |
| | (isopropylamino)nicotinamide |
| CPD-565 | 2-((2-(2-(7-(6-((6-acetyl-8-cyclopentyl-5-methyl-7-oxo-7,8-dihydropyrido[2,3- |
| | d]pyrimidin-2-yl)amino)pyridin-3-yl)-2,7-diazaspiro[4.4]nonan-2-yl)acetyl)-2- |
| | azaspiro[3.3]heptan-6-yl)amino)-N-(6,7-dihydro-5H-pyrrolo[1,2-a]imidazol-2- |
| | yl)-6-(isopropylamino)nicotinamide |
| CPD-566 | 2-((6-(2-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)acetamido)spiro[3.3]heptan- |
| | 2-yl)amino)-N-(6,7-dihydro-5H-pyrrolo[1,2-a]imidazol-2-yl)-6- |
| | (isopropylamino)nicotinamide |
| CPD-567 | 2-((2-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)-4,4,4-trifluorobutanoyl)-2- |
| | azaspiro[3.3]heptan-6-yl)amino)-N-(6,7-dihydro-5H-pyrrolo[1,2-a]imidazol-2- |
| | yl)-6-((methyl-d₃)amino)nicotinamide |
| CPD-568 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6,7-dihydro-5H-pyrrolo[1,2- |
| | a]imidazol-2-yl)-6-(isopropylamino)nicotinamide |
| CPD-569 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6,7-dihydro-5H-pyrrolo[1,2- |
| | a]imidazol-2-yl)-6-((methyl-d₃)amino)nicotinamide |
| CPD-570 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-6- |
| | azaspiro[3.4]octan-2-yl)amino)-N-(6-(dimethylamino)pyridazin-3-yl)-6- |
| | (isopropylamino)nicotinamide |
| CPD-571 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-6-(trifluoromethyl)nicotinamide |
| CPD-572 | 2-((4-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1-carbonyl)-4- |
| | fluorocyclohexyl)amino)-N-(6-(dimethylamino)pyridazin-3-yl)-6- |
| | (isopropylamino)nicotinamide |
| CPD-573 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-6-cyclopropyl-N-(6- |
| | (dimethylamino)pyridazin-3-yl)nicotinamide |
| CPD-574 | 2-((cis-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)-4-hydroxycyclohexyl)amino)-N-(6- |
| | (dimethylamino)pyridazin-3-yl)-6-(isopropylamino)nicotinamide |
| CPD-575 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)-4-hydroxycyclohexyl)amino)-N-(6- |
| | (dimethylamino)pyridazin-3-yl)-6-(isopropylamino)nicotinamide |
| CPD-576 | 2-((trans-4-(3-(4-(6-((6-acetyl-8-cyclopentyl-5-methyl-7-oxo-7,8- |
| | dihydropyrido[2,3-d]pyrimidin-2-yl)amino)pyridin-3-yl)piperazin-1- |
| | yl)pyrrolidine-1-carbonyl)cyclohexyl)amino)-N-(6-(dimethylamino)pyridazin-3- |
| | yl)-6-methylnicotinamide |
| CPD-577 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(6-(dimethylamino)pyridazin-3-yl)- |
| | 6-ethylnicotinamide |
| CPD-578 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(6-(dimethylamino)pyridazin-3-yl)- |
| | 6-ethylnicotinamide |
| CPD-579 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(6-(dimethylamino)pyridazin-3-yl)- |
| | 6-isopropylnicotinamide |
| CPD-580 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(6-(dimethylamino)pyridazin-3-yl)- |
| | 6-isopropylnicotinamide |
| CPD-581 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-6-cyclopropyl-N-(6-(dimethylamino)pyridazin-3- |
| | yl)nicotinamide |
| CPD-582 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(6-(dimethylamino)pyridazin-3-yl)-6- |
| | ethylnicotinamide |
| CPD-583 | 2-((2-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)acetyl)-2- |
| | azaspiro[4.4]nonan-7-yl)amino)-N-(6-(dimethylamino)pyridazin-3-yl)-6- |
| | isopropylnicotinamide |
| CPD-584 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(5,6-dihydro-4H-pyrrolo[1,2- |
| | b]pyrazol-2-yl)-6-(isopropylamino)nicotinamide |
| CPD-585 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)propanamido)spiro[3.3]heptan-2-yl)amino)-N-(6-(dimethylamino)pyridazin-3- |
| | yl)-6-(isopropylamino)nicotinamide |
| CPD-586 | 2-((6-(2-(4-(1-(6-((6-acetyl-8-cyclopentyl-5-methyl-7-oxo-7,8-dihydropyrido[2,3- |
| | d]pyrimidin-2-yl)amino)pyridin-3-yl)ethyl)piperazin-1- |
| | yl)acetamido)spiro[3.3]heptan-2-yl)amino)-N-(6-(dimethylamino)pyridazin-3-yl)- |
| | 6-(isopropylamino)nicotinamide |
| CPD-587 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(1,5-dimethyl-1H-pyrazol-3-yl)-6- |
| | (isopropylamino)nicotinamide |
| CPD-588 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(2-cyclopropyl-1-methyl-1H- |
| | imidazol-4-yl)-6-(isopropylamino)nicotinamide |
| CPD-589 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(2-cyclopropyl-1-methyl-1H- |
| | imidazol-4-yl)-6-(isopropylamino)nicotinamide |
| CPD-590 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(6-(dimethylamino)pyridazin-3-yl)- |
| | 6-methylnicotinamide |
| CPD-591 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-6-(isopropylamino)-N-(5- |
| | methylisoxazol-3-yl)nicotinamide |
| CPD-592 | 2-((3-(3-(4-(6-((6-acetyl-8-cyclopentyl-5-methyl-7-oxo-7,8-dihydropyrido[2,3- |
| | d]pyrimidin-2-yl)amino)pyridin-3-yl)piperazin-1-yl)pyrrolidine-1- |
| | carbonyl)bicyclo[1.1.1]pentan-1-yl)amino)-N-(5,6-dihydro-4H-pyrrolo[1,2- |
| | b]pyrazol-2-yl)-6-(isopropylamino)nicotinamide |
| CPD-593 | 2-((6-(2-(1-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-4- |
| | yl)acetamido)spiro[3.3]heptan-2-yl)amino)-N-(6-(dimethylamino)pyridazin-3-yl)- |
| | 6-(isopropylamino)nicotinamide |
| CPD-594 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1-yl)-N- |
| | methylacetamido)spiro[3.3]heptan-2-yl)amino)-N-(6-(dimethylamino)pyridazin- |
| | 3-yl)-6-(isopropylamino)nicotinamide |
| CPD-595 | 2-((6-((1-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-4- |
| | yl)(methyl)carbamoyl)spiro[3.3]heptan-2-yl)amino)-N-(6- |
| | (dimethylamino)pyridazin-3-yl)-6-(isopropylamino)nicotinamide |
| CPD-596 | 2-((6-(2-(1-((6-((6-acetyl-8-cyclopentyl-5-methyl-7-oxo-7,8-dihydropyrido[2,3- |
| | d]pyrimidin-2-yl)amino)pyridin-3-yl)methyl)pyrrolidin-3- |
| | yl)acetamido)spiro[3.3]heptan-2-yl)amino)-N-(6-(dimethylamino)pyridazin-3-yl)- |
| | 6-(isopropylamino)nicotinamide |
| CPD-597 | 2-((trans-4-(3-(4-(4-((6-acetyl-8-cyclopentyl-5-methyl-7-oxo-7,8- |
| | dihydropyrido[2,3-d]pyrimidin-2-yl)amino)cyclohexyl)piperazin-1-yl)pyrrolidine- |
| | 1-carbonyl)cyclohexyl)amino)-N-(6-(dimethylamino)pyridazin-3-yl)-6- |
| | ethylnicotinamide |
| CPD-598 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperazin-1- |
| | yl)acetamido)spiro[3.3]heptan-2-yl)amino)-N-(6-(dimethylamino)pyridazin-3-yl)- |
| | 4-ethylbenzamide |
| CPD-599 | 2-((6-(2-(4-((6-((6-acetyl-8-cyclopentyl-5-methyl-7-oxo-7,8-dihydropyrido[2,3- |
| | d]pyrimidin-2-yl)amino)pyridin-3-yl)methyl)piperidin-1- |
| | yl)acetamido)spiro[3.3]heptan-2-yl)amino)-N-(6-(dimethylamino)pyridazin-3-yl)- |
| | 6-(isopropylamino)nicotinamide |
| CPD-600 | N-(6-(dimethylamino)pyridazin-3-yl)-2-((3-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-3-oxopropyl)amino)-6-(methylamino)nicotinamide |
| CPD-601 | N-(6-(dimethylamino)pyridazin-3-yl)-2-((4-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-4-oxobutyl)amino)-6-(methylamino)nicotinamide |
| CPD-602 | N-(6-(dimethylamino)pyridazin-3-yl)-2-((5-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-5-oxopentyl)amino)-6-(methylamino)nicotinamide |
| CPD-603 | N-(6-(dimethylamino)pyridazin-3-yl)-2-((6-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-6-oxohexyl)amino)-6-(methylamino)nicotinamide |
| CPD-604 | N-(6-(dimethylamino)pyridazin-3-yl)-2-((7-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-7-oxoheptyl)amino)-6-(methylamino)nicotinamide |
| CPD-605 | N-(6-(dimethylamino)pyridazin-3-yl)-2-((8-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-8-oxooctyl)amino)-6-(methylamino)nicotinamide |
| CPD-606 | N-(6-(dimethylamino)pyridazin-3-yl)-2-((2-(3-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-3-oxopropoxy)ethyl)amino)-6- |
| | (methylamino)nicotinamide |
| CPD-607 | N-(6-(dimethylamino)pyridazin-3-yl)-2-((2-(2-(3-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-3-oxopropoxy)ethoxy)ethyl)amino)-6- |
| | (methylamino)nicotinamide |
| CPD-608 | N-(6-(dimethylamino)pyridazin-3-yl)-2-((2-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)ethyl)amino)-6-(methylamino)nicotinamide |
| CPD-609 | N-(6-(dimethylamino)pyridazin-3-yl)-2-((3-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)propyl)amino)-6-(methylamino)nicotinamide |
| CPD-610 | N-(6-(dimethylamino)pyridazin-3-yl)-2-((4-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)butyl)amino)-6-(methylamino)nicotinamide |
| CPD-611 | N-(6-(dimethylamino)pyridazin-3-yl)-2-((5-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)pentyl)amino)-6-(methylamino)nicotinamide |
| CPD-612 | N-(6-(dimethylamino)pyridazin-3-yl)-2-((6-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)hexyl)amino)-6-(methylamino)nicotinamide |
| CPD-613 | N-(6-(dimethylamino)pyridazin-3-yl)-2-((7-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)heptyl)amino)-6-(methylamino)nicotinamide |
| CPD-614 | N-(6-(dimethylamino)pyridazin-3-yl)-2-((8-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)octyl)amino)-6-(methylamino)nicotinamide |
| CPD-615 | N-(6-(dimethylamino)pyridazin-3-yl)-2-((2-(2-(2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)acetamido)ethoxy)ethyl)amino)-6- |
| | (methylamino)nicotinamide |
| CPD-616 | 7-cyclopentyl-2-((5-(4-(3-((3-((6-(dimethylamino)pyridazin-3-yl)carbamoyl)-6- |
| | (methylamino)pyridin-2-yl)amino)propanoyl)piperazin-1-yl)pyridin-2-yl)amino)- |
| | N,N-dimethyl-7H-pyrrolo[2,3-d]pyrimidine-6-carboxamide |
| CPD-617 | 7-cyclopentyl-2-((5-(4-(4-((3-((6-(dimethylamino)pyridazin-3-yl)carbamoyl)-6- |
| | (methylamino)pyridin-2-yl)amino)butanoyl)piperazin-1-yl)pyridin-2-yl)amino)- |
| | N,N-dimethyl-7H-pyrrolo[2,3-d]pyrimidine-6-carboxamide |
| CPD-618 | 7-cyclopentyl-2-((5-(4-(5-((3-((6-(dimethylamino)pyridazin-3-yl)carbamoyl)-6- |
| | (methylamino)pyridin-2-yl)amino)pentanoyl)piperazin-1-yl)pyridin-2-yl)amino)- |
| | N,N-dimethyl-7H-pyrrolo[2,3-d]pyrimidine-6-carboxamide |
| CPD-619 | 7-cyclopentyl-2-((5-(4-(6-((3-((6-(dimethylamino)pyridazin-3-yl)carbamoyl)-6- |
| | (methylamino)pyridin-2-yl)amino)hexanoyl)piperazin-1-yl)pyridin-2-yl)amino)- |
| | N,N-dimethyl-7H-pyrrolo[2,3-d]pyrimidine-6-carboxamide |
| CPD-620 | 7-cyclopentyl-2-((5-(4-(7-((3-((6-(dimethylamino)pyridazin-3-yl)carbamoyl)-6- |
| | (methylamino)pyridin-2-yl)amino)heptanoyl)piperazin-1-yl)pyridin-2-yl)amino)- |
| | N,N-dimethyl-7H-pyrrolo[2,3-d]pyrimidine-6-carboxamide |
| CPD-621 | 7-cyclopentyl-2-((5-(4-(8-((3-((6-(dimethylamino)pyridazin-3-yl)carbamoyl)-6- |
| | (methylamino)pyridin-2-yl)amino)octanoyl)piperazin-1-yl)pyridin-2-yl)amino)- |
| | N,N-dimethyl-7H-pyrrolo[2,3-d]pyrimidine-6-carboxamide |
| CPD-622 | 7-cyclopentyl-2-((5-(4-(3-(2-((3-((6-(dimethylamino)pyridazin-3-yl)carbamoyl)- |
| | 6-(methylamino)pyridin-2-yl)amino)ethoxy)propanoyl)piperazin-1-yl)pyridin-2- |
| | yl)amino)-N,N-dimethyl-7H-pyrrolo[2,3-d]pyrimidine-6-carboxamide |
| CPD-623 | 7-cyclopentyl-2-((5-(4-(3-(2-(2-((3-((6-(dimethylamino)pyridazin-3- |
| | yl)carbamoyl)-6-(methylamino)pyridin-2- |
| | yl)amino)ethoxy)ethoxy)propanoyl)piperazin-1-yl)pyridin-2-yl)amino)-N,N- |
| | dimethyl-7H-pyrrolo[2,3-d]pyrimidine-6-carboxamide |
| CPD-624 | 7-cyclopentyl-2-((5-(4-(2-((2-((3-((6-(dimethylamino)pyridazin-3-yl)carbamoyl)- |
| | 6-(methylamino)pyridin-2-yl)amino)ethyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-625 | 7-cyclopentyl-2-((5-(4-(2-((3-((3-((6-(dimethylamino)pyridazin-3-yl)carbamoyl)- |
| | 6-(methylamino)pyridin-2-yl)amino)propyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-626 | 7-cyclopentyl-2-((5-(4-(2-((4-((3-((6-(dimethylamino)pyridazin-3-yl)carbamoyl)- |
| | 6-(methylamino)pyridin-2-yl)amino)butyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-627 | 7-cyclopentyl-2-((5-(4-(2-((5-((3-((6-(dimethylamino)pyridazin-3-yl)carbamoyl)- |
| | 6-(methylamino)pyridin-2-yl)amino)pentyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-628 | 7-cyclopentyl-2-((5-(4-(2-((6-((3-((6-(dimethylamino)pyridazin-3-yl)carbamoyl)- |
| | 6-(methylamino)pyridin-2-yl)amino)hexyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-629 | 7-cyclopentyl-2-((5-(4-(2-((7-((3-((6-(dimethylamino)pyridazin-3-yl)carbamoyl)- |
| | 6-(methylamino)pyridin-2-yl)amino)heptyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-630 | 7-cyclopentyl-2-((5-(4-(2-((8-((3-((6-(dimethylamino)pyridazin-3-yl)carbamoyl)- |
| | 6-(methylamino)pyridin-2-yl)amino)octyl)amino)-2-oxoethyl)piperazin-1- |
| | yl)pyridin-2-yl)amino)-N,N-dimethyl-7H-pyrrolo[2,3-d]pyrimidine-6- |
| | carboxamide |
| CPD-631 | 7-cyclopentyl-2-((5-(4-(2-((2-(2-((3-((6-(dimethylamino)pyridazin-3- |
| | yl)carbamoyl)-6-(methylamino)pyridin-2-yl)amino)ethoxy)ethyl)amino)-2- |
| | oxoethyl)piperazin-1-yl)pyridin-2-yl)amino)-N,N-dimethyl-7H-pyrrolo[2,3- |
| | d]pyrimidine-6-carboxamide |
| CPD-632 | N-(6-(dimethylamino)pyridazin-3-yl)-6-(methylamino)-2-((3-oxo-3-(4-(6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)propyl)amino)nicotinamide |
| CPD-633 | N-(6-(dimethylamino)pyridazin-3-yl)-6-(methylamino)-2-((4-oxo-4-(4-(6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)butyl)amino)nicotinamide |
| CPD-634 | N-(6-(dimethylamino)pyridazin-3-yl)-6-(methylamino)-2-((5-oxo-5-(4-(6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)pentyl)amino)nicotinamide |
| CPD-635 | N-(6-(dimethylamino)pyridazin-3-yl)-6-(methylamino)-2-((6-oxo-6-(4-(6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)hexyl)amino)nicotinamide |
| CPD-636 | N-(6-(dimethylamino)pyridazin-3-yl)-6-(methylamino)-2-((7-oxo-7-(4-(6-((6′- |
| | oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)heptyl)amino)nicotinamide |
| CPD-637 | N-(6-(dimethylamino)pyridazin-3-yl)-6-(methylamino)-2-((8-oxo-8-(4-(6-((6′- |
| | ox0-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1-yl)octyl)amino)nicotinamide |
| CPD-638 | N-(6-(dimethylamino)pyridazin-3-yl)-6-(methylamino)-2-((2-(3-oxo-3-(4-(6-((6′- |
| | oxo-7′,8′-dihydro-6′H-spiro[cyclohexane-1,9′-pyrazino[1′,2′: 1,5]pyrrolo[2,3- |
| | d]pyrimidin]-2′-yl)amino)pyridin-3-yl)piperazin-1- |
| | yl)propoxy)ethyl)amino)nicotinamide |
| CPD-639 | N-(6-(dimethylamino)pyridazin-3-yl)-6-(methylamino)-2-((2-(2-(3-oxo-3-(4-(6- |
| | ((6′-oxo-7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)propoxy)ethoxy)ethyl)amino)nicotinamide |
| CPD-640 | N-(6-(dimethylamino)pyridazin-3-yl)-6-(methylamino)-2-((2-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)ethyl)amino)nicotinamide |
| CPD-641 | N-(6-(dimethylamino)pyridazin-3-yl)-6-(methylamino)-2-((3-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)propyl)amino)nicotinamide |
| CPD-642 | N-(6-(dimethylamino)pyridazin-3-yl)-6-(methylamino)-2-((4-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)butyl)amino)nicotinamide |
| CPD-643 | N-(6-(dimethylamino)pyridazin-3-yl)-6-(methylamino)-2-((5-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)pentyl)amino)nicotinamide |
| CPD-644 | N-(6-(dimethylamino)pyridazin-3-yl)-6-(methylamino)-2-((6-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1 - |
| | yl)acetamido)hexyl)amino)nicotinamide |
| CPD-645 | N-(6-(dimethylamino)pyridazin-3-yl)-6-(methylamino)-2-((7-(2-(4-(6-((6′-oxo- |
| | 7′,8′-dihydro-6′H-spiro[cyclohexane-1,9′-pyrazino[11,2′:1,5]pyrrolo[2,3- |
| | d]pyrimidin]-2′-yl)amino)pyridin-3-yl)piperazin-1- |
| | yl)acetamido)heptyl)amino)nicotinamide |
| CPD-646 | N-(6-(dimethylamino)pyridazin-3-yl)-6-(methylamino)-2-((8-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)octyl)amino)nicotinamide |
| CPD-647 | N-(6-(dimethylamino)pyridazin-3-yl)-6-(methylamino)-2-((2-(2-(2-(4-(6-((6′-oxo- |
| | 7′,8′-dihydro-6′H-spiro[cyclohexane-1,9′-pyrazino[1′,2′:1,5]pyrrolo[2,3- |
| | d]pyrimidin]-2′-yl)amino)pyridin-3-yl)piperazin-1- |
| | yl)acetamido)ethoxy)ethyl)amino)nicotinamide |
| CPD-648 | N-(6-(dimethylamino)pyridazin-3-yl)-2-((3-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-3-oxopropyl)amino)-6-(methylamino)nicotinamide |
| CPD-649 | N-(6-(dimethylamino)pyridazin-3-yl)-2-((4-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-4-oxobutyl)amino)-6-(methylamino)nicotinamide |
| CPD-650 | N-(6-(dimethylamino)pyridazin-3-yl)-2-((5-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-5-oxopentyl)amino)-6-(methylamino)nicotinamide |
| CPD-651 | N-(6-(dimethylamino)pyridazin-3-yl)-2-((6-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-6-oxohexyl)amino)-6-(methylamino)nicotinamide |
| CPD-652 | N-(6-(dimethylamino)pyridazin-3-yl)-2-((7-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-7-oxoheptyl)amino)-6-(methylamino)nicotinamide |
| CPD-653 | N-(6-(dimethylamino)pyridazin-3-yl)-2-((8-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-8-oxooctyl)amino)-6-(methylamino)nicotinamide |
| CPD-654 | N-(6-(dimethylamino)pyridazin-3-yl)-2-((2-(3-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-3-oxopropoxy)ethyl)amino)-6-(methylamino)nicotinamide |
| CPD-655 | N-(6-(dimethylamino)pyridazin-3-yl)-2-((2-(2-(3-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)-3-oxopropoxy)ethoxy)ethyl)amino)-6- |
| | (methylamino)nicotinamide |
| CPD-656 | N-(6-(dimethylamino)pyridazin-3-yl)-2-((2-(4-((6-((5-fluoro-4-(4-fluoro-1- |
| | isopropyl-2-methyl-1H-benzo[d]imidazol-6-yl)pyrimidin-2-yl)amino)pyridin-3- |
| | yl)methyl)piperazin-1-yl)-2-oxoethyl)amino)-6-(methylamino)nicotinamide |
| CPD-657 | 7-cyclopentyl-2-((5-(4-((3-((6-(dimethylamino)pyridazin-3-yl)carbamoyl)-6- |
| | (methylamino)pyridin-2-yl)glycyl)piperazin-1-yl)pyridin-2-yl)amino)-N,N- |
| | dimethyl-7H-pyrrolo[2,3-d]pyrimidine-6-carboxamide |
| CPD-658 | N-(6-(dimethylamino)pyridazin-3-yl)-2-((2-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)ethyl)amino)-6-(methylamino)nicotinamide |
| CPD-659 | N-(6-(dimethylamino)pyridazin-3-yl)-2-((3-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)propyl)amino)-6-(methylamino)nicotinamide |
| CPD-660 | N-(6-(dimethylamino)pyridazin-3-yl)-2-((4-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)butyl)amino)-6-(methylamino)nicotinamide |
| CPD-661 | N-(6-(dimethylamino)pyridazin-3-yl)-2-((5-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)pentyl)amino)-6-(methylamino)nicotinamide |
| CPD-662 | N-(6-(dimethylamino)pyridazin-3-yl)-2-((6-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)hexyl)amino)-6-(methylamino)nicotinamide |
| CPD-663 | N-(6-(dimethylamino)pyridazin-3-yl)-2-((7-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)heptyl)amino)-6-(methylamino)nicotinamide |
| CPD-664 | N-(6-(dimethylamino)pyridazin-3-yl)-2-((8-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)octyl)amino)-6-(methylamino)nicotinamide |
| CPD-665 | N-(6-(dimethylamino)pyridazin-3-yl)-2-((2-(2-(2-(4-(6-((5-fluoro-4-(4-fluoro-1- |
| | isopropyl-2-methyl-1H-benzo[d]imidazol-6-yl)pyrimidin-2-yl)amino)pyridin-3- |
| | yl)piperazin-1-yl)acetamido)ethoxy)ethyl)amino)-6-(methylamino)nicotinamide |
| CPD-666 | 2-((3-((4-((8-cyclopentyl-7-oxo-7,8-dihydropyrido[2,3-d]pyrimidin-2- |
| | yl)amino)cyclohexyl)amino)-3-oxopropyl)amino)-N-(6- |
| | (dimethylamino)pyridazin-3-yl)-6-(methylamino)nicotinamide |
| CPD-667 | 2-((4-((4-((8-cyclopentyl-7-oxo-7,8-dihydropyrido[2,3-d]pyrimidin-2- |
| | yl)amino)cyclohexyl)amino)-4-oxobutyl)amino)-N-(6-(dimethylamino)pyridazin- |
| | 3-yl)-6-(methylamino)nicotinamide |
| CPD-668 | 2-((5-((4-((8-cyclopentyl-7-oxo-7,8-dihydropyrido[2,3-d]pyrimidin-2- |
| | yl)amino)cyclohexyl)amino)-5-oxopentyl)amino)-N-(6- |
| | (dimethylamino)pyridazin-3-yl)-6-(methylamino)nicotinamide |
| CPD-669 | 2-((6-((4-((8-cyclopentyl-7-oxo-7,8-dihydropyrido[2,3-d]pyrimidin-2- |
| | yl)amino)cyclohexyl)amino)-6-oxohexyl)amino)-N-(6-(dimethylamino)pyridazin- |
| | 3-yl)-6-(methylamino)nicotinamide |
| CPD-670 | 2-((7-((4-((8-cyclopentyl-7-oxo-7,8-dihydropyrido[2,3-d]pyrimidin-2- |
| | yl)amino)cyclohexyl)amino)-7-oxoheptyl)amino)-N-(6- |
| | (dimethylamino)pyridazin-3-yl)-6-(methylamino)nicotinamide |
| CPD-671 | 2-((8-((4-((8-cyclopentyl-7-oxo-7,8-dihydropyrido[2,3-d]pyrimidin-2- |
| | yl)amino)cyclohexyl)amino)-8-oxooctyl)amino)-N-(6-(dimethylamino)pyridazin- |
| | 3-yl)-6-(methylamino)nicotinamide |
| CPD-672 | 2-((2-(3-((4-((8-cyclopentyl-7-oxo-7,8-dihydropyrido[2,3-d]pyrimidin-2- |
| | yl)amino)cyclohexyl)amino)-3-oxopropoxy)ethyl)amino)-N-(6- |
| | (dimethylamino)pyridazin-3-yl)-6-(methylamino)nicotinamide |
| CPD-673 | 2-((3-((2-((8-cyclopentyl-7-oxo-7,8-dihydropyrido[2,3-d]pyrimidin-2- |
| | yl)amino)spiro[3.5]nonan-7-yl)amino)-3-oxopropyl)amino)-N-(6- |
| | (dimethylamino)pyridazin-3-yl)-6-(methylamino)nicotinamide |
| CPD-674 | 2-((4-((2-((8-cyclopentyl-7-oxo-7,8-dihydropyrido[2,3-d]pyrimidin-2- |
| | yl)amino)spiro[3.5]nonan-7-yl)amino)-4-oxobutyl)amino)-N-(6- |
| | (dimethylamino)pyridazin-3-yl)-6-(methylamino)nicotinamide |
| CPD-675 | 2-((5-((2-((8-cyclopentyl-7-oxo-7,8-dihydropyrido[2,3-d]pyrimidin-2- |
| | yl)amino)spiro[3.5]nonan-7-yl)amino)-5-oxopentyl)amino)-N-(6- |
| | (dimethylamino)pyridazin-3-yl)-6-(methylamino)nicotinamide |
| CPD-676 | 2-((6-((2-((8-cyclopentyl-7-oxo-7,8-dihydropyrido[2,3-d]pyrimidin-2- |
| | yl)amino)spiro[3.5]nonan-7-yl)amino)-6-oxohexyl)amino)-N-(6- |
| | (dimethylamino)pyridazin-3-yl)-6-(methylamino)nicotinamide |
| CPD-677 | 2-((7-((2-((8-cyclopentyl-7-oxo-7,8-dihydropyrido[2,3-d]pyrimidin-2- |
| | yl)amino)spiro[3.5]nonan-7-yl)amino)-7-oxoheptyl)amino)-N-(6- |
| | (dimethylamino)pyridazin-3-yl)-6-(methylamino)nicotinamide |
| CPD-678 | 2-((8-((2-((8-cyclopentyl-7-oxo-7,8-dihydropyrido[2,3-d]pyrimidin-2- |
| | yl)amino)spiro[3.5]nonan-7-yl)amino)-8-oxooctyl)amino)-N-(6- |
| | (dimethylamino)pyridazin-3-yl)-6-(methylamino)nicotinamide |
| CPD-679 | 2-((2-(3-((2-((8-cyclopentyl-7-oxo-7,8-dihydropyrido[2,3-d]pyrimidin-2- |
| | yl)amino)spiro[3.5]nonan-7-yl)amino)-3-oxopropoxy)ethyl)amino)-N-(6- |
| | (dimethylamino)pyridazin-3-yl)-6-(methylamino)nicotinamide |

Enumerated embodiments F1 to F679 relate to compounds exemplified compound in Tables 4A and 4B, or a pharmaceutically acceptable salts thereof, as well as a pharmaceutical composition of such a compound or salt.

F1. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propyl)amino)-N-(4,5-dimethylthiazol-2-yl)-6-methylnicotinamide, or a pharmaceutically acceptable salt thereof.
F2. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-methoxypyridazin-3-yl)-6-methylnicotinamide, or a pharmaceutically acceptable salt thereof.
F3. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4,5-dimethylthiazol-2-yl)-6-methylnicotinamide, or a pharmaceutically acceptable salt thereof.
F4. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-6-(methylamino)nicotinamide, or a pharmaceutically acceptable salt thereof.
F5. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(methylamino)nicotinamide, or a pharmaceutically acceptable salt thereof.
F6. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-cyclopropyl-5-methylpyridin-2-yl)-6-(methylamino)nicotinamide, or a pharmaceutically acceptable salt thereof.
F7. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-5-methylpyridin-2-yl)-6-(methylamino)nicotinamide, or a pharmaceutically acceptable salt thereof.
F8. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-(dimethylamino)pyridazin-3-yl)-6-methylnicotinamide, or a pharmaceutically acceptable salt thereof.
F9. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-cyclopropyl-5-methylpyridin-2-yl)-6-methylnicotinamide, or a pharmaceutically acceptable salt thereof.
F10. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-(dimethylamino)pyridazin-3-yl)-6-(methylamino)nicotinamide, or a pharmaceutically acceptable salt thereof.
F11. A heterobifunctional compound of 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-methoxypyridazin-3-yl)-6-(methylamino)nicotinamide, or a pharmaceutically acceptable salt thereof.
F12. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-fluorobenzamide, or a pharmaceutically acceptable salt thereof.
F13. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-(trifluoromethyl)benzamide, or a pharmaceutically acceptable salt thereof.
F14. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)pentyl)amino)-N-(6-(dimethylamino)pyridazin-3-yl)-6-(methylamino)nicotinamide, or a pharmaceutically acceptable salt thereof.
F15. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-isopropylpyridazin-3-yl)-6-(methylamino)nicotinamide, or a pharmaceutically acceptable salt thereof.
F16. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-methylnicotinamide, or a pharmaceutically acceptable salt thereof.
F17. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(pyrrolidin-1-yl)pyridazin-3-yl)nicotinamide, or a pharmaceutically acceptable salt thereof.
F18. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(isopropylamino)nicotinamide, or a pharmaceutically acceptable salt thereof.
F19. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(trifluoromethyl)pyridazin-3-yl)nicotinamide, or a pharmaceutically acceptable salt thereof.
F20. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-chloropyridin-2-yl)-6-(methylamino)nicotinamide, or a pharmaceutically acceptable salt thereof.
F21. A heterobifunctional compound of 2-((5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)pentyl)amino)-N-(6-(dimethylamino)pyridazin-3-yl)-6-(methylamino)nicotinamide, or a pharmaceutically acceptable salt thereof.
F22. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-N-(6-(dimethylamino)pyridazin-3-yl)-6-(methylamino)nicotinamide, or a pharmaceutically acceptable salt thereof.
F23. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)(methyl)amino)-N-(6-(dimethylamino)pyridazin-3-yl)-6-(methylamino)nicotinamide, or a pharmaceutically acceptable salt thereof.
F24. A heterobifunctional compound of 2-((1-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)-1H-pyrazol-4-yl)amino)-N-(6-(dimethylamino)pyridazin-3-yl)-6-(methylamino)nicotinamide, or a pharmaceutically acceptable salt thereof.
F25. A heterobifunctional compound of 2-((2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)-2H-1,2,3-triazol-4-yl)amino)-N-(6-(dimethylamino)pyridazin-3-yl)-6-(methylamino)nicotinamide, or a pharmaceutically acceptable salt thereof.
F26. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)isoxazol-5-yl)amino)-N-(6-(dimethylamino)pyridazin-3-yl)-6-(methylamino)nicotinamide, or a pharmaceutically acceptable salt thereof.
F27. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyclopropyl-N-(6-(dimethylamino)pyridazin-3-yl)nicotinamide, or a pharmaceutically acceptable salt thereof.
F28. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyano-N-(6-(dimethylamino)pyridazin-3-yl)nicotinamide, or a pharmaceutically acceptable salt thereof.
F29. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)hexan-2-yl)amino)-N-(6-(dimethylamino)pyridazin-3-yl)-6-(methylamino)nicotinamide, or a pharmaceutically acceptable salt thereof.
F30. A heterobifunctional compound of 2-((4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)butyl)amino)-N-(6-(dimethylamino)pyridazin-3-yl)-6-(methylamino)nicotinamide, or a pharmaceutically acceptable salt thereof.
F31. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridazin-3-yl)-6-(methylamino)nicotinamide, or a pharmaceutically acceptable salt thereof.
F32. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrazolo[4,3-b]pyridin-5-yl)-6-(methylamino)nicotinamide, or a pharmaceutically acceptable salt thereof.
F33. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrrolo[3,2-b]pyridin-5-yl)-6-(methylamino)nicotinamide, or a pharmaceutically acceptable salt thereof.
F34. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(6-(dimethylamino)pyridazin-3-yl)-6-(methylamino)nicotinamide, or a pharmaceutically acceptable salt thereof.
F35 4-(6-((6-acetyl-8-cyclopentyl-5-methyl-7-oxo-7,8-dihydropyrido[2,3-d]pyrimidin-2-yl)amino)pyridin-3-yl)-N-(2-(3-((3-((6-(dimethylamino)pyridazin-3-yl)carbamoyl)-6-(methylamino)pyridin-2-yl)amino)cyclobutyl)ethyl)piperazine-1-carboxamide, or a pharmaceutically acceptable salt thereof.
F36. A heterobifunctional compound of 2-((5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-5-oxopentyl)amino)-N-(6-(dimethylamino)pyridazin-3-yl)-6-(methylamino)nicotinamide, or a pharmaceutically acceptable salt thereof.
F37. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)butyl)amino)-N-(6-(dimethylamino)pyridazin-3-yl)-6-(methylamino)nicotinamide, or a pharmaceutically acceptable salt thereof.
F38. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-methyl-3H-imidazo[4,5-b]pyridin-5-yl)-6-(methylamino)nicotinamide, or a pharmaceutically acceptable salt thereof.
F39. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-(methylamino)nicotinamide, or a pharmaceutically acceptable salt thereof.
F40. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6,7-dihydro-5H-cyclopenta[b]pyridin-2-yl)-6-(methylamino)nicotinamide, or a pharmaceutically acceptable salt thereof.
F41. A heterobifunctional compound of 2-((2-(3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)ethyl)amino)-N-(6-(dimethylamino)pyridazin-3-yl)-6-(methylamino)nicotinamide, or a pharmaceutically acceptable salt thereof.
F42 2-(4-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)piperidin-1-yl)-N-(6-(dimethylamino)pyridazin-3-yl)-6-(methylamino)nicotinamide, or a pharmaceutically acceptable salt thereof.
F43. A heterobifunctional compound of 2-((trans-4-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclohexyl)amino)-N-(6-(dimethylamino)pyridazin-3-yl)-6-(methylamino)nicotinamide, or a pharmaceutically acceptable salt thereof.
F44. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyclopropyl-N-(6-cyclopropyl-5-methylpyridin-2-yl)nicotinamide, or a pharmaceutically acceptable salt thereof.
F45. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-cyclopropyl-N-(6-cyclopropyl-5-methylpyridin-2-yl)nicotinamide, or a pharmaceutically acceptable salt thereof.
F46. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-(dimethylamino)phenyl)-6-(methylamino)nicotinamide, or a pharmaceutically acceptable salt thereof.
F47. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-(dimethylamino)pyridin-2-yl)-6-(methylamino)nicotinamide, or a pharmaceutically acceptable salt thereof.
F48. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-(dimethylamino)cyclohexyl)-6-(methylamino)nicotinamide, or a pharmaceutically acceptable salt thereof.
F49. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrazol-3-yl)-6-(methylamino)nicotinamide, or a pharmaceutically acceptable salt thereof.
F50. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1,5-dimethyl-1H-pyrazol-3-yl)-6-(methylamino)nicotinamide, or a pharmaceutically acceptable salt thereof.
F51. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridin-2-yl)-6-(methylamino)nicotinamide, or a pharmaceutically acceptable salt thereof.
F52. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridin-2-yl)-6-(methylamino)nicotinamide, or a pharmaceutically acceptable salt thereof.
F53. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-4-methylpyridin-2-yl)-6-(methylamino)nicotinamide, or a pharmaceutically acceptable salt thereof.
F54. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-ethylnicotinamide, or a pharmaceutically acceptable salt thereof.
F55. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-(dimethylamino)cyclopentyl)-6-(methylamino)nicotinamide, or a pharmaceutically acceptable salt thereof.
F56. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-cyclopropyl-5-methylthiazol-2-yl)-6-(methylamino)nicotinamide, or a pharmaceutically acceptable salt thereof.
F57. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-methylpyridin-2-yl)-6-(methylamino)nicotinamide, or a pharmaceutically acceptable salt thereof.
F58. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridin-3-yl)-6-(methylamino)nicotinamide, or a pharmaceutically acceptable salt thereof.
F59. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(cyclobutylamino)-N-(6-(dimethylamino)pyridazin-3-yl)nicotinamide, or a pharmaceutically acceptable salt thereof.
F60. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-6-cyclopropyl-N-(6-cyclopropyl-5-methylpyridin-2-yl)nicotinamide, or a pharmaceutically acceptable salt thereof.
F61. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-3-methylpyridin-2-yl)-6-(methylamino)nicotinamide, or a pharmaceutically acceptable salt thereof.
F62. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F63. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-isopropylnicotinamide, or a pharmaceutically acceptable salt thereof.
F64. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-(dimethylamino)cyclobutyl)-6-(methylamino)nicotinamide, or a pharmaceutically acceptable salt thereof.
F65. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-((dimethylamino)methyl)cyclobutyl)-6-(methylamino)nicotinamide, or a pharmaceutically acceptable salt thereof.
F66. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-((methyl-d3)amino)nicotinamide, or a pharmaceutically acceptable salt thereof.
F67. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)spiro[3.3]heptan-2-yl)-6-(methylamino)nicotinamide, or a pharmaceutically acceptable salt thereof.
F68. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(trifluoromethyl)nicotinamide, or a pharmaceutically acceptable salt thereof.
F69. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6,7-dihydro-5H-cyclopenta[b]pyridin-2-yl)-6-(methylamino)nicotinamide, or a pharmaceutically acceptable salt thereof.
F70. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-(methylamino)nicotinamide, or a pharmaceutically acceptable salt thereof.
F71. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-N-methyl-6-(methylamino)nicotinamide, or a pharmaceutically acceptable salt thereof.
F72. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(methylamino)nicotinamide, or a pharmaceutically acceptable salt thereof.
F73. A heterobifunctional compound of 2-((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-(dimethylamino)pyridazin-3-yl)-6-(methylamino)nicotinamide, or a pharmaceutically acceptable salt thereof.
F74. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-3-yl)-6-(methylamino)nicotinamide, or a pharmaceutically acceptable salt thereof.
F75. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-methylpyridin-2-yl)nicotinamide, or a pharmaceutically acceptable salt thereof.
F76. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-isopropylpyridin-2-yl)-6-(methylamino)nicotinamide, or a pharmaceutically acceptable salt thereof.
F77. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-oxo-1,6-dihydropyridazin-3-yl)nicotinamide, or a pharmaceutically acceptable salt thereof.
F78. A heterobifunctional compound of 2-((6-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)spiro[3.3]heptan-2-yl)amino)-N-(6-(dimethylamino)pyridazin-3-yl)-6-(methylamino)nicotinamide, or a pharmaceutically acceptable salt thereof.
F79. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-(methylamino)nicotinamide, or a pharmaceutically acceptable salt thereof.
F80. A heterobifunctional compound of 2-((3-((3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)methyl)cyclobutyl)amino)-N-(6-(dimethylamino)pyridazin-3-yl)-6-(methylamino)nicotinamide, or a pharmaceutically acceptable salt thereof.
F81 4-(6-((6-acetyl-8-cyclopentyl-5-methyl-7-oxo-7,8-dihydropyrido[2,3-d]pyrimidin-2-yl)amino)pyridin-3-yl)-N-((6-((3-((6-(dimethylamino)pyridazin-3-yl)carbamoyl)-6-(methylamino)pyridin-2-yl)amino)spiro[3.3]heptan-2-yl)methyl)piperazine-1-carboxamide, or a pharmaceutically acceptable salt thereof.
F82. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)spiro[3.3]heptan-2-yl)amino)-N-(6-(dimethylamino)pyridazin-3-yl)-6-(methylamino)nicotinamide, or a pharmaceutically acceptable salt thereof.
F83. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propyl)spiro[3.3]heptan-2-yl)amino)-N-(6-(dimethylamino)pyridazin-3-yl)-6-(methylamino)nicotinamide, or a pharmaceutically acceptable salt thereof.
F84. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)spiro[3.3]heptan-2-yl)amino)-N-(6-(dimethylamino)pyridazin-3-yl)-6-(methylamino)nicotinamide, or a pharmaceutically acceptable salt thereof.
F85. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(tert-butylamino)-N-(6-(dimethylamino)pyridazin-3-yl)nicotinamide, or a pharmaceutically acceptable salt thereof.
F86. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropylpyridin-4-yl)-6-(methylamino)nicotinamide, or a pharmaceutically acceptable salt thereof.
F87. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-(trifluoromethyl)pyridin-2-yl)-6-(methylamino)nicotinamide, or a pharmaceutically acceptable salt thereof.
F88. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridin-2-yl)-6-(methylamino)nicotinamide, or a pharmaceutically acceptable salt thereof.
F89. A heterobifunctional compound of 2-((2-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)ethyl)amino)-N-(6-(dimethylamino)pyridazin-3-yl)-6-(methylamino)nicotinamide, or a pharmaceutically acceptable salt thereof.
F90. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyanopyridin-2-yl)-6-(methylamino)nicotinamide, or a pharmaceutically acceptable salt thereof.
F91. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-(trifluoromethyl)pyridin-2-yl)nicotinamide, or a pharmaceutically acceptable salt thereof.
F92. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methyl-1H-pyrazol-3-yl)-6-(methylamino)nicotinamide, or a pharmaceutically acceptable salt thereof.
F93. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-3-yl)-6-(methylamino)nicotinamide, or a pharmaceutically acceptable salt thereof.
F94. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-4-yl)-6-(methylamino)nicotinamide, or a pharmaceutically acceptable salt thereof.
F95. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-isopropyl-1-methyl-1H-pyrazol-3-yl)-6-(methylamino)nicotinamide, or a pharmaceutically acceptable salt thereof.
F96. A heterobifunctional compound of 2-((6-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)spiro[3.3]heptan-2-yl)amino)-N-(6-(dimethylamino)pyridazin-3-yl)-6-(methylamino)nicotinamide, or a pharmaceutically acceptable salt thereof.
F97. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F98. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyclopropyl-1-methyl-1H-pyrazol-3-yl)-6-(methylamino)nicotinamide, or a pharmaceutically acceptable salt thereof.
F99. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-isopropyl-5-methyl-1H-pyrazol-3-yl)-6-(methylamino)nicotinamide, or a pharmaceutically acceptable salt thereof.
F100. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(1H-pyrazol-3-yl)nicotinamide, or a pharmaceutically acceptable salt thereof.
F101. A heterobifunctional compound of 2-((3-(A heterobifunctional compound of 2-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-2-oxoethyl)cyclobutyl)amino)-N-(6-(dimethylamino)pyridazin-3-yl)-6-(methylamino)nicotinamide, or a pharmaceutically acceptable salt thereof.
F102. A heterobifunctional compound of 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)azetidin-3-yl)propyl)amino)-N-(6-(dimethylamino)pyridazin-3-yl)-6-(methylamino)nicotinamide, or a pharmaceutically acceptable salt thereof.
F103. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-cyclopropylpyridin-2-yl)-6-(methylamino)nicotinamide, or a pharmaceutically acceptable salt thereof.
F104. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-aminopyridazin-3-yl)-6-(methylamino)nicotinamide, or a pharmaceutically acceptable salt thereof.
F105. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-methylpyridazin-3-yl)nicotinamide, or a pharmaceutically acceptable salt thereof.
F106. A heterobifunctional compound of 2-((1-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)pyrrolidin-3-yl)amino)-N-(6-(dimethylamino)pyridazin-3-yl)-6-(methylamino)nicotinamide, or a pharmaceutically acceptable salt thereof.
F107. A heterobifunctional compound of 2-((1-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)azetidin-3-yl)amino)-N-(6-(dimethylamino)pyridazin-3-yl)-6-(methylamino)nicotinamide, or a pharmaceutically acceptable salt thereof.
F108. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-methoxypyridazin-3-yl)-6-(methylamino)nicotinamide, or a pharmaceutically acceptable salt thereof.
F109. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5,6-dimethylpyridin-2-yl)-6-(methylamino)nicotinamide, or a pharmaceutically acceptable salt thereof.
F110. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(methylamino)pyridazin-3-yl)nicotinamide, or a pharmaceutically acceptable salt thereof.
F111. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-isopropyl-5-methylpyridin-2-yl)-6-(methylamino)nicotinamide, or a pharmaceutically acceptable salt thereof.
F112. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-chloro-6-cyclopropylpyridin-2-yl)-6-(methylamino)nicotinamide, or a pharmaceutically acceptable salt thereof.
F113. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-5-fluoropyridin-2-yl)-6-(methylamino)nicotinamide, or a pharmaceutically acceptable salt thereof.
F114. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-3-fluoropyridin-2-yl)-6-(methylamino)nicotinamide, or a pharmaceutically acceptable salt thereof.
F115. A heterobifunctional compound of 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F116. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-methoxy-5-methylpyridin-2-yl)-6-(methylamino)nicotinamide, or a pharmaceutically acceptable salt thereof.
F117. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F118. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-dimethylpropanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F119. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-isopropylnicotinamide, or a pharmaceutically acceptable salt thereof.
F120. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-methylnicotinamide, or a pharmaceutically acceptable salt thereof.
F121. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(trifluoromethyl)nicotinamide, or a pharmaceutically acceptable salt thereof.
F122. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-oxo-1,6-dihydropyridin-2-yl)nicotinamide, or a pharmaceutically acceptable salt thereof.
F123. A heterobifunctional compound of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-methyl-6-oxo-1,6-dihydropyridin-2-yl)-6-(methylamino)nicotinamide, or a pharmaceutically acceptable salt thereof.
F124. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-ethylnicotinamide, or a pharmaceutically acceptable salt thereof.
F125. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-methoxypyridin-2-yl)-6-(methylamino)nicotinamide, or a pharmaceutically acceptable salt thereof.
F126. A heterobifunctional compound of 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-6-(methylamino)-N-(6-methylpyridin-2-yl)nicotinamide, or a pharmaceutically acceptable salt thereof.
F127. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-5-(trifluoromethyl)-1H-pyrazol-3-yl)-6-(methylamino)nicotinamide, or a pharmaceutically acceptable salt thereof.
F128. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-fluoropyridin-2-yl)-6-(methylamino)nicotinamide, or a pharmaceutically acceptable salt thereof.
F129. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1,5-dimethyl-1H-pyrazol-3-yl)-6-(methylamino)nicotinamide, or a pharmaceutically acceptable salt thereof.
F130. A heterobifunctional compound of 2-((4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)amino)-N-(6-(dimethylamino)pyridazin-3-yl)-6-(methylamino)nicotinamide, or a pharmaceutically acceptable salt thereof.
F131. A heterobifunctional compound of 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(1,5-dimethyl-1H-pyrazol-3-yl)-6-(methylamino)nicotinamide, or a pharmaceutically acceptable salt thereof.
F132. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyano-6-cyclopropylpyridin-2-yl)-6-(methylamino)nicotinamide, or a pharmaceutically acceptable salt thereof.
F133. A heterobifunctional compound of 2-((2-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F134 4-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(methylamino)pyridin-2-yl)amino)spiro[3.3]heptan-2-yl)piperazine-1-carboxamide, or a pharmaceutically acceptable salt thereof.
F135. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(methylamino)nicotinamide, or a pharmaceutically acceptable salt thereof.
F136. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclopentyl)amino)-N-(6-(dimethylamino)pyridazin-3-yl)-6-(methylamino)nicotinamide, or a pharmaceutically acceptable salt thereof.
F137. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(pyridin-2-yl)nicotinamide, or a pharmaceutically acceptable salt thereof.
F138. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-cyclopropyl-N-(6-cyclopropylpyridin-2-yl)nicotinamide, or a pharmaceutically acceptable salt thereof.
F139. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(isopropylamino)nicotinamide, or a pharmaceutically acceptable salt thereof.
F140. A heterobifunctional compound of 2-((6-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)spiro[3.3]heptan-2-yl)amino)-N-(6-(dimethylamino)pyridazin-3-yl)-6-(methylamino)nicotinamide, or a pharmaceutically acceptable salt thereof.
F141. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-cyano-N-(6-cyclopropylpyridin-2-yl)nicotinamide, or a pharmaceutically acceptable salt thereof.
F142 6-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-6′-cyano-N-(4,5-dimethylthiazol-2-yl)-[2,3′-bipyridine]-5-carboxamide, or a pharmaceutically acceptable salt thereof.
F143. A heterobifunctional compound of 2-((7-(2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetamido)heptyl)amino)-N-(6-(dimethylamino)pyridazin-3-yl)-6-(methylamino)nicotinamide, or a pharmaceutically acceptable salt thereof.
F144. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1-methyl-1H-pyrazol-3-yl)-6-(methylamino)nicotinamide, or a pharmaceutically acceptable salt thereof.
F145. A heterobifunctional compound of 2-(((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)methyl)amino)-N-(6-(dimethylamino)pyridazin-3-yl)-6-(methylamino)nicotinamide, or a pharmaceutically acceptable salt thereof.

F146. A heterobifunctional compound of 2-(((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)methyl)amino)-N-(6-(dimethylamino)pyridazin-3-yl)-6-(methylamino)nicotinamide, or a pharmaceutically acceptable salt thereof.

F147. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)spiro[3.3]heptan-2-yl)amino)-N-(6-(dimethylamino)pyridazin-3-yl)-6-(methylamino)nicotinamide, or a pharmaceutically acceptable salt thereof.
F148. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)cyclobutyl)amino)-N-(6-(dimethylamino)pyridazin-3-yl)-6-(methylamino)nicotinamide, or a pharmaceutically acceptable salt thereof.
F149. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-(methylamino)nicotinamide, or a pharmaceutically acceptable salt thereof.
F150. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(methylamino)nicotinamide, or a pharmaceutically acceptable salt thereof.
F151 4-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(methylamino)pyridin-2-yl)amino)cyclobutyl)piperazine-1-carboxamide, or a pharmaceutically acceptable salt thereof.
F152. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-imidazol-4-yl)-6-(methylamino)nicotinamide, or a pharmaceutically acceptable salt thereof.
F153. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-5-yl)-6-(methylamino)nicotinamide, or a pharmaceutically acceptable salt thereof.
F154. A heterobifunctional compound of 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-cyclopropylpyridin-2-yl)-6-(methylamino)nicotinamide, or a pharmaceutically acceptable salt thereof.
F155. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-6-(methylamino)-N-(6-methylpyridin-2-yl)nicotinamide, or a pharmaceutically acceptable salt thereof.
F156. A heterobifunctional compound of 2-((3-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)cyclobutyl)amino)-N-(6-(dimethylamino)pyridazin-3-yl)-6-(methylamino)nicotinamide, or a pharmaceutically acceptable salt thereof.
F157. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamoyl)cyclobutyl)amino)-N-(6-(dimethylamino)pyridazin-3-yl)-6-(methylamino)nicotinamide, or a pharmaceutically acceptable salt thereof.
F158. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.4]octan-6-yl)amino)-N-(6-(dimethylamino)pyridazin-3-yl)-6-(methylamino)nicotinamide, or a pharmaceutically acceptable salt thereof.
F159. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(isoxazol-3-yl)-6-(methylamino)nicotinamide, or a pharmaceutically acceptable salt thereof.
F160. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(5-methylisoxazol-3-yl)nicotinamide, or a pharmaceutically acceptable salt thereof.
F161. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-methoxypyridin-2-yl)-6-(methylamino)nicotinamide, or a pharmaceutically acceptable salt thereof.
F162. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-cyclopropylpyridin-2-yl)-6-(methylamino)nicotinamide, or a pharmaceutically acceptable salt thereof.
F163. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-((methyl-d3)amino)nicotinamide, or a pharmaceutically acceptable salt thereof.
F164. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)methyl)cyclobutyl)amino)-N-(6-(dimethylamino)pyridazin-3-yl)-6-(methylamino)nicotinamide, or a pharmaceutically acceptable salt thereof.
F165. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-(methylamino)nicotinamide, or a pharmaceutically acceptable salt thereof.
F166 4-(6-((6-acetyl-8-cyclopentyl-5-methyl-7-oxo-7,8-dihydropyrido[2,3-d]pyrimidin-2-yl)amino)pyridin-3-yl)-N-((3-((3-((6-(dimethylamino)pyridazin-3-yl)carbamoyl)-6-(methylamino)pyridin-2-yl)amino)cyclobutyl)methyl)piperazine-1-carboxamide, or a pharmaceutically acceptable salt thereof.
F167. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)methyl)cyclobutyl)amino)-N-(6-(dimethylamino)pyridazin-3-yl)-6-(methylamino)nicotinamide, or a pharmaceutically acceptable salt thereof.
F168. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)cyclobutyl)amino)-N-(6-(dimethylamino)pyridazin-3-yl)-6-(methylamino)nicotinamide, or a pharmaceutically acceptable salt thereof.
F169 4-(6-((6-acetyl-8-cyclopentyl-5-methyl-7-oxo-7,8-dihydropyrido[2,3-d]pyrimidin-2-yl)amino)pyridin-3-yl)-N-((trans-4-(((3-((6-(dimethylamino)pyridazin-3-yl)carbamoyl)-6-(methylamino)pyridin-2-yl)amino)methyl)cyclohexyl)methyl)piperazine-1-carboxamide, or a pharmaceutically acceptable salt thereof.
F170. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(methylamino)-N-(6-methylpyridin-2-yl)nicotinamide, or a pharmaceutically acceptable salt thereof.
F171. A heterobifunctional compound of 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-6-(methylamino)-N-(6-methylpyridin-2-yl)nicotinamide, or a pharmaceutically acceptable salt thereof.
F172. A heterobifunctional compound of 2-((3-((1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carboxamido)methyl)cyclobutyl)amino)-N-(6-(dimethylamino)pyridazin-3-yl)-6-(methylamino)nicotinamide, or a pharmaceutically acceptable salt thereof.
F173. A heterobifunctional compound of 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane- 1-carbonyl)-2-azaspiro[3.3]heptan-6-yl)amino)-N-(6-cyclopropylpyridin-2-yl)-6-(methylamino)nicotinamide, or a pharmaceutically acceptable salt thereof.
F174. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)spiro[3.3]heptan-2-yl)amino)-N-(6-(dimethylamino)pyridazin-3-yl)-6-(methylamino)nicotinamide, or a pharmaceutically acceptable salt thereof.
F175. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoyl)-2-azaspiro[3.3]heptan-6-yl)amino)-N-(6-cyclopropylpyridin-2-yl)-6-(methylamino)nicotinamide, or a pharmaceutically acceptable salt thereof.
F176. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoyl)-2-azaspiro[3.3]heptan-6-yl)amino)-6-(methylamino)-N-(6-methylpyridin-2-yl)nicotinamide, or a pharmaceutically acceptable salt thereof.
F177. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-(dimethylamino)pyridin-2-yl)-6-(methylamino)nicotinamide, or a pharmaceutically acceptable salt thereof.
F178. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)cyclobutyl)amino)-N-(6-(dimethylamino)pyridazin-3-yl)-6-(methylamino)nicotinamide, or a pharmaceutically acceptable salt thereof.
F179. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,2-dimethyl-1H-imidazol-4-yl)-6-(methylamino)nicotinamide, or a pharmaceutically acceptable salt thereof.
F180. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-aminopyridin-2-yl)-6-(methylamino)nicotinamide, or a pharmaceutically acceptable salt thereof.
F181. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-(methylamino)pyridin-2-yl)nicotinamide, or a pharmaceutically acceptable salt thereof.
F182. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5,6-dimethylpyridin-2-yl)-6-(methylamino)nicotinamide, or a pharmaceutically acceptable salt thereof.
F183. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrrol-3-yl)-6-(methylamino)nicotinamide, or a pharmaceutically acceptable salt thereof.
F184. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(difluoromethyl)pyridin-2-yl)-6-(methylamino)nicotinamide, or a pharmaceutically acceptable salt thereof.
F185. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)cyclobutyl)amino)-N-(6-(dimethylamino)pyridazin-3-yl)-6-(methylamino)nicotinamide, or a pharmaceutically acceptable salt thereof.
F186. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(6-(dimethylamino)pyridazin-3-yl)-6-(methylamino)nicotinamide, or a pharmaceutically acceptable salt thereof.
F187. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)azetidin-3-yl)amino)-N-(6-(dimethylamino)pyridazin-3-yl)-6-(methylamino)nicotinamide, or a pharmaceutically acceptable salt thereof.
F188. A heterobifunctional compound of 2-(((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)methyl)amino)-N-(6-(dimethylamino)pyridazin-3-yl)-6-(methylamino)nicotinamide, or a pharmaceutically acceptable salt thereof.
F189. A heterobifunctional compound of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-1-oxopropan-2-yl)oxy)spiro[3.3]heptan-2-yl)amino)-N-(6-(dimethylamino)pyridazin-3-yl)-6-(methylamino)nicotinamide, or a pharmaceutically acceptable salt thereof.
F190. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(6-methylpyridin-2-yl)nicotinamide, or a pharmaceutically acceptable salt thereof.
F191. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-methyl-N-(6-methylpyridin-2-yl)nicotinamide, or a pharmaceutically acceptable salt thereof.
F192. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-methyl-N-(5-methylisoxazol-3-yl)nicotinamide, or a pharmaceutically acceptable salt thereof.
F193. A heterobifunctional compound of 2-(((cis-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-(dimethylamino)pyridazin-3-yl)-6-(methylamino)nicotinamide, or a pharmaceutically acceptable salt thereof.
F194 2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)-N-(6-(dimethylamino)pyridazin-3-yl)-6-(methylamino)nicotinamide, or a pharmaceutically acceptable salt thereof.
F195. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-isopropyl-1-methyl-1H-imidazol-4-yl)-6-(methylamino)nicotinamide, or a pharmaceutically acceptable salt thereof.
F196. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-6-(methylamino)-N-(6-methylpyridin-2-yl)nicotinamide, or a pharmaceutically acceptable salt thereof.
F197. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(1,5-dimethyl-1H-pyrazol-3-yl)-6-(methylamino)nicotinamide, or a pharmaceutically acceptable salt thereof.
F198. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-6-(methylamino)-N-(5-methylisoxazol-3-yl)nicotinamide, or a pharmaceutically acceptable salt thereof.
F199. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(5-methylisoxazol-3-yl)nicotinamide, or a pharmaceutically acceptable salt thereof.
F200. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-(methylamino)nicotinamide, or a pharmaceutically acceptable salt thereof.
F201. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)pyrrolidin-3-yl)amino)-N-(6-(dimethylamino)pyridazin-3-yl)-6-(methylamino)nicotinamide, or a pharmaceutically acceptable salt thereof.
F202. A heterobifunctional compound of 2-((3-((4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)methyl)cyclobutyl)amino)-N-(6-(dimethylamino)pyridazin-3-yl)-6-(methylamino)nicotinamide, or a pharmaceutically acceptable salt thereof.
F203. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(6-(dimethylamino)pyridazin-3-yl)-6-(methylamino)nicotinamide, or a pharmaceutically acceptable salt thereof.
F204. A heterobifunctional compound of 2-(((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)cyclohexyl)methyl)amino)-N-(6-(dimethylamino)pyridazin-3-yl)-6-(methylamino)nicotinamide, or a pharmaceutically acceptable salt thereof.
F205. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-(isopropylamino)nicotinamide, or a pharmaceutically acceptable salt thereof.
F206. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-((methyl-d3)amino)nicotinamide, or a pharmaceutically acceptable salt thereof.
F207. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-methylnicotinamide, or a pharmaceutically acceptable salt thereof.
F208. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F209. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-methylnicotinamide, or a pharmaceutically acceptable salt thereof.
F210. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-ethylnicotinamide, or a pharmaceutically acceptable salt thereof.
F211. A heterobifunctional compound of 2-((1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)azetidin-3-yl)amino)-N-(6-(dimethylamino)pyridazin-3-yl)-6-(methylamino)nicotinamide, or a pharmaceutically acceptable salt thereof.
F212. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-H-pyrazol-3-yl)-6-isopropylnicotinamide, or a pharmaceutically acceptable salt thereof.
F213. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-cyclopropyl-N-(1,5-dimethyl-1H-pyrazol-3-yl)nicotinamide, or a pharmaceutically acceptable salt thereof.
F214. A heterobifunctional compound of 2-(((4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)cyclohexyl)methyl)amino)-N-(6-(dimethylamino)pyridazin-3-yl)-6-(methylamino)nicotinamide, or a pharmaceutically acceptable salt thereof.
F215. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyclopropylisoxazol-3-yl)-6-(methylamino)nicotinamide, or a pharmaceutically acceptable salt thereof.
F216. A heterobifunctional compound of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-N-(6-(dimethylamino)pyridazin-3-yl)-6-(methylamino)nicotinamide, or a pharmaceutically acceptable salt thereof.
F217. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-(methylamino)nicotinamide, or a pharmaceutically acceptable salt thereof.
F218. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(6-(dimethylamino)pyridazin-3-yl)-6-(methylamino)nicotinamide, or a pharmaceutically acceptable salt thereof.
F219. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(6-(dimethylamino)pyridazin-3-yl)-6-(methylamino)nicotinamide, or a pharmaceutically acceptable salt thereof.
F220. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methoxy-6-methylpyridin-2-yl)-6-(methylamino)nicotinamide, or a pharmaceutically acceptable salt thereof.
F221. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-fluoro-6-methylpyridin-2-yl)-6-(methylamino)nicotinamide, or a pharmaceutically acceptable salt thereof.
F222. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(isopropylamino)nicotinamide, or a pharmaceutically acceptable salt thereof.
F223. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclohexyl)amino)-N-(6-(dimethylamino)pyridazin-3-yl)-6-(methylamino)nicotinamide, or a pharmaceutically acceptable salt thereof.
F224. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F225. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-((methyl-d3)amino)-N-(5-methylisoxazol-3-yl)nicotinamide, or a pharmaceutically acceptable salt thereof.
F226. A heterobifunctional compound of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-6-((methyl-d3)amino)-N-(6-methylpyridin-2-yl)nicotinamide, or a pharmaceutically acceptable salt thereof.
F227. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-isopropylisoxazol-3-yl)-6-(methylamino)nicotinamide, or a pharmaceutically acceptable salt thereof.
F228. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(1,5-dimethyl-1H-pyrazol-3-yl)-6-(methylamino)nicotinamide, or a pharmaceutically acceptable salt thereof.
F229. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-(methylamino)-N-(5-methylisoxazol-3-yl)nicotinamide, or a pharmaceutically acceptable salt thereof.
F230. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(1,5-dimethyl-1H-pyrazol-3-yl)-6-((methyl-d3)amino)nicotinamide, or a pharmaceutically acceptable salt thereof.
F231. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-((methyl-d3)amino)-N-(5-methylisoxazol-3-yl)nicotinamide, or a pharmaceutically acceptable salt thereof.
F232. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-((methyl-d3)amino)-N-(6-methylpyridin-2-yl)nicotinamide, or a pharmaceutically acceptable salt thereof.
F233. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-N-(1,5-dimethyl-1H-pyrazol-3-yl)-6-((methyl-d3)amino)nicotinamide, or a pharmaceutically acceptable salt thereof.
F234. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-6-((methyl-d3)amino)-N-(5-methylisoxazol-3-yl)nicotinamide, or a pharmaceutically acceptable salt thereof.
F235. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-2-azaspiro[3.4]octan-6-yl)amino)-N-(6-(dimethylamino)pyridazin-3-yl)-6-(methylamino)nicotinamide, or a pharmaceutically acceptable salt thereof.
F236. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(2-cyclopropyl-1-methyl-1H-imidazol-4-yl)-6-((methyl-d3)amino)nicotinamide, or a pharmaceutically acceptable salt thereof.
F237. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-((methyl-d3)amino)nicotinamide, or a pharmaceutically acceptable salt thereof.
F238. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-isopropyl-2-methyl-1H-imidazol-4-yl)-6-(methylamino)nicotinamide, or a pharmaceutically acceptable salt thereof.
F239. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-fluoropyridin-2-yl)-6-((methyl-d3)amino)nicotinamide, or a pharmaceutically acceptable salt thereof.
F240. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-cyclopropylpyridin-2-yl)-6-(methylamino)nicotinamide, or a pharmaceutically acceptable salt thereof.
F241. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-((methyl-d3)amino)nicotinamide, or a pharmaceutically acceptable salt thereof.
F242. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(methylamino)nicotinamide, or a pharmaceutically acceptable salt thereof.
F243. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)cyclobutyl)amino)-N-(6-(dimethylamino)pyridazin-3-yl)-6-(methylamino)nicotinamide, or a pharmaceutically acceptable salt thereof.
F244. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-((methyl-d3)amino)nicotinamide, or a pharmaceutically acceptable salt thereof.
F245. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-((methyl-d3)amino)nicotinamide, or a pharmaceutically acceptable salt thereof.
F246. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(5-methyloxazol-2-yl)nicotinamide, or a pharmaceutically acceptable salt thereof.
F247. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(4,5-dimethyloxazol-2-yl)-6-(methylamino)nicotinamide, or a pharmaceutically acceptable salt thereof.
F248. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-N-(6-(dimethylamino)pyridazin-3-yl)-6-((methyl-d3)amino)nicotinamide, or a pharmaceutically acceptable salt thereof.
F249. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F250. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-((methyl-d3)amino)nicotinamide, or a pharmaceutically acceptable salt thereof.
F251 6-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-1H-pyrrolo[2,3-b]pyridine-5-carboxamide, or a pharmaceutically acceptable salt thereof.
F252. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-cyclopropylpyridin-2-yl)-6-((methyl-d3)amino)nicotinamide, or a pharmaceutically acceptable salt thereof.
F253. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-(isopropylamino)nicotinamide, or a pharmaceutically acceptable salt thereof.
F254. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-(methylamino)nicotinamide, or a pharmaceutically acceptable salt thereof.
F255. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-4-chloro-N-(6-cyclopropyl-5-methylpyridin-2-yl)benzamide, or a pharmaceutically acceptable salt thereof.
F256 4-(6-((6-acetyl-8-cyclopentyl-5-methyl-7-oxo-7,8-dihydropyrido[2,3-d]pyrimidin-2-yl)amino)pyridin-3-yl)-N-(2-(3-((5-chloro- A heterobifunctional compound of 2-((6-cyclopropyl-5-methylpyridin-2-yl)carbamoyl)phenyl)amino)cyclobutyl)ethyl)piperazine-1-carboxamide, or a pharmaceutically acceptable salt thereof.
F257. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-4-chloro-N-(6-(dimethylamino)pyridazin-3-yl)benzamide, or a pharmaceutically acceptable salt thereof.
F258. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(6-(dimethylamino)pyridazin-3-yl)-4-fluorobenzamide, or a pharmaceutically acceptable salt thereof.
F259. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-chloro-N-(5-methylisoxazol-3-yl)benzamide, or a pharmaceutically acceptable salt thereof.
F260. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-fluoro-N-(5-methylisoxazol-3-yl)benzamide, or a pharmaceutically acceptable salt thereof.
F261. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-4-(trifluoromethyl)benzamide, or a pharmaceutically acceptable salt thereof.
F262. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-5-(trifluoromethyl)benzamide, or a pharmaceutically acceptable salt thereof.
F263. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-5-fluorobenzamide, or a pharmaceutically acceptable salt thereof.
F264. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-4-fluorobenzamide, or a pharmaceutically acceptable salt thereof.
F265. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-fluorobenzamide, or a pharmaceutically acceptable salt thereof.
F266. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-(trifluoromethyl)benzamide, or a pharmaceutically acceptable salt thereof.
F267. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F268. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F269. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)azetidin-3-yl)amino)-N-(6-(dimethylamino)pyridazin-3-yl)-6-(methylamino)nicotinamide, or a pharmaceutically acceptable salt thereof.
F270. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-((methyl-d3)amino)-N-(6-methylpyridin-2-yl)nicotinamide, or a pharmaceutically acceptable salt thereof.
F271. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclopentyl)amino)-N-(6-(dimethylamino)pyridazin-3-yl)-6-(methylamino)nicotinamide, or a pharmaceutically acceptable salt thereof.
F272. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F273. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(2-cyclopropyl-1-methyl-1H-imidazol-4-yl)-6-((methyl-d3)amino)nicotinamide, or a pharmaceutically acceptable salt thereof.
F274. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(2-cyclopropyl-1-methyl-1H-imidazol-4-yl)-6-(isopropylamino)nicotinamide, or a pharmaceutically acceptable salt thereof.
F275. A heterobifunctional compound of 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(2-cyclopropyl-1-methyl-1H-imidazol-4-yl)-6-(isopropylamino)nicotinamide, or a pharmaceutically acceptable salt thereof.
F276. A heterobifunctional compound of 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(2-cyclopropyl-1-methyl-1H-imidazol-4-yl)-6-((methyl-d3)amino)nicotinamide, or a pharmaceutically acceptable salt thereof.
F277. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-(isopropylamino)nicotinamide, or a pharmaceutically acceptable salt thereof.
F278. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-((methyl-d3)amino)nicotinamide, or a pharmaceutically acceptable salt thereof.
F279. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(2-cyclopropyl-1-methyl-1H-imidazol-4-yl)-6-(isopropylamino)nicotinamide, or a pharmaceutically acceptable salt thereof.
F280. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-(isopropylamino)nicotinamide, or a pharmaceutically acceptable salt thereof.
F281. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(methylamino)-N-(5-methylisoxazol-3-yl)nicotinamide, or a pharmaceutically acceptable salt thereof.
F282. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(isopropylamino)-N-(5-methylisoxazol-3-yl)nicotinamide, or a pharmaceutically acceptable salt thereof.
F283. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-((methyl-d3)amino)-N-(5-methylisoxazol-3-yl)nicotinamide, or a pharmaceutically acceptable salt thereof.
F284. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(2-cyclopropyl-1-methyl-1H-imidazol-4-yl)-6-(isopropylamino)nicotinamide, or a pharmaceutically acceptable salt thereof.
F285. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(2-cyclopropyl-1-methyl-1H-imidazol-4-yl)-6-((methyl-d3)amino)nicotinamide, or a pharmaceutically acceptable salt thereof.
F286. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-methyl-N-(5-methylisoxazol-3-yl)benzamide, or a pharmaceutically acceptable salt thereof.
F287. A heterobifunctional compound of 4-(6-((6-acetyl-8-cyclopentyl-5-methyl-7-oxo-7,8-dihydropyrido[2,3-d]pyrimidin-2-yl)amino)pyridin-3-yl)-N-(3-((3-((2-cyclopropyl-1-methyl-1H-imidazol-4-yl)carbamoyl)-6-((methyl-d3)amino)pyridin-2-yl)amino)cyclobutyl)piperazine-1-carboxamide, or a pharmaceutically acceptable salt thereof.
F288. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-(isopropylamino)nicotinamide, or a pharmaceutically acceptable salt thereof.
F289. A heterobifunctional compound oF 4-(6-((6-acetyl-8-cyclopentyl-5-methyl-7-oxo-7,8-dihydropyrido[2,3-d]pyrimidin-2-yl)amino)pyridin-3-yl)-N-(3-((3-((2-cyclopropyl-1-methyl-1H-imidazol-4-yl)carbamoyl)-6-(isopropylamino)pyridin-2-yl)amino)cyclobutyl)piperazine-1-carboxamide, or a pharmaceutically acceptable salt thereof.
F290. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-(isopropylamino)-N-(5-methylisoxazol-3-yl)nicotinamide, or a pharmaceutically acceptable salt thereof.
F291. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(dimethylamino)nicotinamide, or a pharmaceutically acceptable salt thereof.
F292. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-isopropoxynicotinamide, or a pharmaceutically acceptable salt thereof.
F293. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(ethylamino)nicotinamide, or a pharmaceutically acceptable salt thereof.
F294. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-6-((methyl-d3)amino)-N-(5-methylisoxazol-3-yl)nicotinamide, or a pharmaceutically acceptable salt thereof.
F295. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-6-(isopropylamino)-N-(5-methylisoxazol-3-yl)nicotinamide, or a pharmaceutically acceptable salt thereof.
F296. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopentyl-1-methyl-1H-imidazol-4-yl)-6-(methylamino)nicotinamide, or a pharmaceutically acceptable salt thereof.
F297. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-cyclobutyl-5-methyl-1H-pyrazol-3-yl)-6-(methylamino)nicotinamide, or a pharmaceutically acceptable salt thereof.
F298. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(propylamino)nicotinamide, or a pharmaceutically acceptable salt thereof.
F299. A heterobifunctional compound of 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)piperidin-4-yl)amino)-N-(6-(dimethylamino)pyridazin-3-yl)-6-(methylamino)nicotinamide, or a pharmaceutically acceptable salt thereof.
F300. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-(methylamino)nicotinamide, or a pharmaceutically acceptable salt thereof.
F301. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(6-(dimethylamino)pyridazin-3-yl)-6-(isopropylamino)nicotinamide, or a pharmaceutically acceptable salt thereof.
F302. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1,5-dimethyl-1H-pyrazol-3-yl)-6-(isopropylamino)nicotinamide, or a pharmaceutically acceptable salt thereof.
F303. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(6-(dimethylamino)pyridazin-3-yl)-6-(isopropylamino)nicotinamide, or a pharmaceutically acceptable salt thereof.
F304. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(2-cyclopropyl-1-methyl-1H-imidazol-4-yl)-6-(isopropylamino)nicotinamide, or a pharmaceutically acceptable salt thereof.
F305. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F306. A heterobifunctional compound of 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane- 1-carbonyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F307. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-((methyl-d3)amino)nicotinamide, or a pharmaceutically acceptable salt thereof.
F308. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-ethyl-1-methyl-1H-imidazol-4-yl)-6-(methylamino)nicotinamide, or a pharmaceutically acceptable salt thereof.
F309. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(l -methyl-2-propyl-1H-imidazol-4-yl)-6-(methylamino)nicotinamide, or a pharmaceutically acceptable salt thereof.
F310. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclobutyl-1-methyl-1H-imidazol-4-yl)-6-(methylamino)nicotinamide, or a pharmaceutically acceptable salt thereof.
F3 11. A heterobifunctional compound of 3-((3-((6-(dimethylamino)pyridazin-3-yl)carbamoyl)-6-(methylamino)pyridin-2-yl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate, or a pharmaceutically acceptable salt thereof.
F312. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-ethyl-5-methyl-1H-pyrazol-3-yl)-6-(methylamino)nicotinamide, or a pharmaceutically acceptable salt thereof.
F313. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(2-cyclopropyl-1-methyl-1H-imidazol-4-yl)-6-(isopropylamino)nicotinamide, or a pharmaceutically acceptable salt thereof.
F314. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methyl-1-propyl-1H-pyrazol-3-yl)-6-(methylamino)nicotinamide, or a pharmaceutically acceptable salt thereof.
F315. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-cyclopropyl-5-methyl-1H-pyrazol-3-yl)-6-(methylamino)nicotinamide, or a pharmaceutically acceptable salt thereof.
F316. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-(isopropylamino)nicotinamide, or a pharmaceutically acceptable salt thereof.
F317. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)oxy)-N-(2-cyclopropyl-1-methyl-1H-imidazol-4-yl)-6-(isopropylamino)nicotinamide, or a pharmaceutically acceptable salt thereof.
F318. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-isobutylnicotinamide, or a pharmaceutically acceptable salt thereof.
F319. A heterobifunctional compound of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-N-(6-(dimethylamino)pyridazin-3-yl)-6-(methylamino)nicotinamide, or a pharmaceutically acceptable salt thereof.
F320. A heterobifunctional compound of 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(2-cyclopropyl-1-methyl- 1H-imidazol-4-yl)-6-(methylamino)nicotinamide, or a pharmaceutically acceptable salt thereof.
F321. A heterobifunctional compound of 2-((2-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)-2-azaspiro[3.3]heptan-6-yl)amino)-N-(2-cyclopropyl-1-methyl-1H-imidazol-4-yl)-6-(methylamino)nicotinamide, or a pharmaceutically acceptable salt thereof.
F322. A heterobifunctional compound of 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F323. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F324. A heterobifunctional compound of 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F325. A heterobifunctional compound of 2-((2-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F326. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(isopropylamino)nicotinamide, or a pharmaceutically acceptable salt thereof.
F327. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(2-cyclopropyl-1-methyl-1H-imidazol-4-yl)-6-(isopropylamino)nicotinamide, or a pharmaceutically acceptable salt thereof.
F328. A heterobifunctional compound of 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(6-(dimethylamino)pyridazin-3-yl)-6-(methylamino)nicotinamide, or a pharmaceutically acceptable salt thereof.
F329. A heterobifunctional compound of 2-((1-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)piperidin-4-yl)amino)-N-(6-(dimethylamino)pyridazin-3-yl)-6-(methylamino)nicotinamide, or a pharmaceutically acceptable salt thereof.
F330. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-(1-hydroxyethyl)-1-methyl-1H-imidazol-4-yl)-6-(methylamino)nicotinamide, or a pharmaceutically acceptable salt thereof.
F331. A heterobifunctional compound of 2-((3-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonyl)cyclobutyl)amino)-N-(6-(dimethylamino)pyridazin-3-yl)-6-(methylamino)nicotinamide, or a pharmaceutically acceptable salt thereof.
F332. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)cyclobutyl)amino)-N-(6-(dimethylamino)pyridazin-3-yl)-6-(methylamino)nicotinamide, or a pharmaceutically acceptable salt thereof.
F333. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-(methylamino)nicotinamide, or a pharmaceutically acceptable salt thereof.
F334. A heterobifunctional compound of 2-((3-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)cyclobutyl)amino)-N-(6-(dimethylamino)pyridazin-3-yl)-6-(methylamino)nicotinamide, or a pharmaceutically acceptable salt thereof.
F335. A heterobifunctional compound of 2-((3-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)cyclobutyl)amino)-N-(6-(dimethylamino)pyridazin-3-yl)-6-(methylamino)nicotinamide, or a pharmaceutically acceptable salt thereof.
F336. A heterobifunctional compound of 2-(((3-((3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)methyl)cyclobutyl)amino)-N-(6-(dimethylamino)pyridazin-3-yl)-6-(methylamino)nicotinamide, or a pharmaceutically acceptable salt thereof.
F337. A heterobifunctional compound of 2-((3-(9-(6-((6-acetyl-8-cyclopentyl-5-methyl-7-oxo-7,8-dihydropyrido[2,3-d]pyrimidin-2-yl)amino)pyridin-3-yl)-3,9-diazaspiro[5.5]undecane-3-carbonyl)cyclobutyl)amino)-N-(6-(dimethylamino)pyridazin-3-yl)-6-(methylamino)nicotinamide, or a pharmaceutically acceptable salt thereof.
F338. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F339. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(2-cyclopropyl-1-methyl-1H-imidazol-4-yl)-6-((methyl-d3)amino)nicotinamide, or a pharmaceutically acceptable salt thereof.
F340. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-(2-hydroxypropan-2-yl)-1-methyl-1H-imidazol-4-yl)-6-(methylamino)nicotinamide, or a pharmaceutically acceptable salt thereof.
F341. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F342. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F343. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-((methyl-d3)amino)nicotinamide, or a pharmaceutically acceptable salt thereof.
F344. A heterobifunctional compound of 2-(((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-3-fluoroazetidin-3-yl)methyl)amino)-N-(6-(dimethylamino)pyridazin-3-yl)-6-(methylamino)nicotinamide, or a pharmaceutically acceptable salt thereof.
F345. A heterobifunctional compound of 2-(((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)bicyclo[1.1.1]pentan-1-yl)methyl)amino)-N-(6-(dimethylamino)pyridazin-3-yl)-6-(methylamino)nicotinamide, or a pharmaceutically acceptable salt thereof.
F346. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)bicyclo[1.1.1]pentan-1-yl)amino)-N-(6-(dimethylamino)pyridazin-3-yl)-6-(methylamino)nicotinamide, or a pharmaceutically acceptable salt thereof.
F347. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.5]nonan-7-yl)amino)-N-(2-cyclopropyl-1-methyl-1H-imidazol-4-yl)-6-(methylamino)nicotinamide, or a pharmaceutically acceptable salt thereof.
F348. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclobutane-1-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F349. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(1-hydroxyethyl)nicotinamide, or a pharmaceutically acceptable salt thereof.
F350. A heterobifunctional compound of 2-((4-(9-(6-((6-acetyl-8-cyclopentyl-5-methyl-7-oxo-7,8-dihydropyrido[2,3-d]pyrimidin-2-yl)amino)pyridin-3-yl)-3,9-diazaspiro[5.5]undecane-3-carbonyl)cyclohexyl)amino)-N-(6-(dimethylamino)pyridazin-3-yl)-6-(methylamino)nicotinamide, or a pharmaceutically acceptable salt thereof.
F351. A heterobifunctional compound of 2-((4-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonyl)cyclohexyl)amino)-N-(6-(dimethylamino)pyridazin-3-yl)-6-(methylamino)nicotinamide, or a pharmaceutically acceptable salt thereof.
F352. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(2-hydroxypropan-2-yl)nicotinamide, or a pharmaceutically acceptable salt thereof.
F353. A heterobifunctional compound of 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(1,5-dimethyl-1H-pyrazol-3-yl)-6-((methyl-d3)amino)nicotinamide, or a pharmaceutically acceptable salt thereof.
F354. A heterobifunctional compound of 2-(((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-5-oxa-2-azaspiro[3.4]octan-7-yl)methyl)amino)-N-(6-(dimethylamino)pyridazin-3-yl)-6-(methylamino)nicotinamide, or a pharmaceutically acceptable salt thereof.
F355. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)azetidin-3-yl)amino)-N-(6-(dimethylamino)pyridazin-3-yl)-6-(isopropylamino)nicotinamide, or a pharmaceutically acceptable salt thereof.
F356. A heterobifunctional compound of 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)azetidin-3-yl)amino)-N-(6-(dimethylamino)pyridazin-3-yl)-6-(isopropylamino)nicotinamide, or a pharmaceutically acceptable salt thereof.
F357. A heterobifunctional compound of 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)piperidin-4-yl)amino)-N-(6-(dimethylamino)pyridazin-3-yl)-6-(isopropylamino)nicotinamide, or a pharmaceutically acceptable salt thereof.
F358. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-(isopropylamino)nicotinamide, or a pharmaceutically acceptable salt thereof.
F359. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)piperidin-4-yl)amino)-N-(6-(dimethylamino)pyridazin-3-yl)-6-(isopropylamino)nicotinamide, or a pharmaceutically acceptable salt thereof.
F360. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(6-(dimethylamino)pyridazin-3-yl)-6-(isopropylamino)nicotinamide, or a pharmaceutically acceptable salt thereof.
F361. A heterobifunctional compound of 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-((methyl-d3)amino)-N-(5-methylisoxazol-3-yl)nicotinamide, or a pharmaceutically acceptable salt thereof.
F362. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)spiro[3.3]heptan-2-yl)amino)-N-(6-(dimethylamino)pyridazin-3-yl)-6-(methylamino)nicotinamide, or a pharmaceutically acceptable salt thereof.
F363. A heterobifunctional compound of 2-((6-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonyl)spiro[3.3]heptan-2-yl)amino)-N-(6-(dimethylamino)pyridazin-3-yl)-6-(methylamino)nicotinamide, or a pharmaceutically acceptable salt thereof.
F364. A heterobifunctional compound of 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)-2-azaspiro[3.3]heptan-6-yl)amino)-N-(6-(dimethylamino)pyridazin-3-yl)-6-(methylamino)nicotinamide, or a pharmaceutically acceptable salt thereof.
F365. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanamido)cyclobutyl)amino)-N-(6-(dimethylamino)pyridazin-3-yl)-6-(isopropylamino)nicotinamide, or a pharmaceutically acceptable salt thereof.
F366. A heterobifunctional compound of 2-((6-(1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclopropane-1-carboxamido)spiro[3.3]heptan-2-yl)amino)-N-(2-cyclopropyl-1-methyl-1H-imidazol-4-yl)-6-(isopropylamino)nicotinamide, or a pharmaceutically acceptable salt thereof.
F367. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F368. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F369. A heterobifunctional compound of 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-(isopropylamino)nicotinamide, or a pharmaceutically acceptable salt thereof.
F370. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)pyrrolidin-3-yl)amino)-N-(6-(dimethylamino)pyridazin-3-yl)-6-(isopropylamino)nicotinamide, or a pharmaceutically acceptable salt thereof.
F371. A heterobifunctional compound of 2-((6-(1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclopropane-1-carboxamido)spiro[3.3]heptan-2-yl)amino)-6-(isopropylamino)-N-(5-methylisoxazol-3-yl)nicotinamide, or a pharmaceutically acceptable salt thereof.
F372. A heterobifunctional compound of 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)pyrrolidin-3-yl)amino)-N-(6-(dimethylamino)pyridazin-3-yl)-6-(isopropylamino)nicotinamide, or a pharmaceutically acceptable salt thereof.
F373. A heterobifunctional compound of 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octan-2-yl)acetyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F374. A heterobifunctional compound of 2-((6-((7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonan-2-yl)carbamoyl)spiro[3.3]heptan-2-yl)amino)-N-(6-(dimethylamino)pyridazin-3-yl)-6-(methylamino)nicotinamide, or a pharmaceutically acceptable salt thereof.
F375. A heterobifunctional compound of 2-((3-((7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonan-2-yl)carbamoyl)cyclobutyl)amino)-N-(6-(dimethylamino)pyridazin-3-yl)-6-(methylamino)nicotinamide, or a pharmaceutically acceptable salt thereof.
F376. A heterobifunctional compound of 2-((6-(3-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-1-carbonyl)spiro[3.3]heptan-2-yl)amino)-N-(6-(dimethylamino)pyridazin-3-yl)-6-(methylamino)nicotinamide, or a pharmaceutically acceptable salt thereof.
F377. A heterobifunctional compound of 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-(isopropylamino)-N-(5-methylisoxazol-3-yl)nicotinamide, or a pharmaceutically acceptable salt thereof.
F378. A heterobifunctional compound of 2-((6-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octane-2-carbonyl)spiro[3.3]heptan-2-yl)amino)-N-(6-(dimethylamino)pyridazin-3-yl)-6-((methyl-d3)amino)nicotinamide, or a pharmaceutically acceptable salt thereof.
F379. A heterobifunctional compound of 2-((6-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonane-2-carbonyl)spiro[3.3]heptan-2-yl)amino)-N-(6-(dimethylamino)pyridazin-3-yl)-6-((methyl-d3)amino)nicotinamide, or a pharmaceutically acceptable salt thereof.
F380. A heterobifunctional compound of 2-((6-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)spiro[3.3]heptan-2-yl)amino)-N-(6-(dimethylamino)pyridazin-3-yl)-6-((methyl-d3)amino)nicotinamide, or a pharmaceutically acceptable salt thereof.
F381. A heterobifunctional compound of 2-((3-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)cyclobutyl)amino)-N-(6-(dimethylamino)pyridazin-3-yl)-6-(methylamino)nicotinamide, or a pharmaceutically acceptable salt thereof.
F382. A heterobifunctional compound of 2-((3-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)amino)acetamido)cyclobutyl)amino)-N-(6-(dimethylamino)pyridazin-3-yl)-6-(isopropylamino)nicotinamide, or a pharmaceutically acceptable salt thereof.
F383. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-6-azaspiro[2.5]octan-1-yl)amino)-N-(6-(dimethylamino)pyridazin-3-yl)-6-((methyl-d3)amino)nicotinamide, or a pharmaceutically acceptable salt thereof.
F384. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F385. A heterobifunctional compound of 2-((4-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)cyclohexyl)amino)-N-(6-(dimethylamino)pyridazin-3-yl)-6-(methylamino)nicotinamide, or a pharmaceutically acceptable salt thereof.
F386. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperidin-1-yl)acetyl)piperidin-4-yl)amino)-N-(6-(dimethylamino)pyridazin-3-yl)-6-(isopropylamino)nicotinamide, or a pharmaceutically acceptable salt thereof.
F387. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propan-2-yl)cyclobutyl)amino)-N-(1,5-dimethyl-1H-pyrazol-3-yl)-6-(isopropylamino)nicotinamide, or a pharmaceutically acceptable salt thereof.
F388. A heterobifunctional compound of 2-((3-((3-(6-((6-acetyl-8-cyclopentyl-5-methyl-7-oxo-7,8-dihydropyrido[2,3-d]pyrimidin-2-yl)amino)pyridin-3-yl)-3-azaspiro[5.5]undecan-9-yl)carbamoyl)cyclobutyl)amino)-N-(6-(dimethylamino)pyridazin-3-yl)-6-(methylamino)nicotinamide, or a pharmaceutically acceptable salt thereof.
F389. A heterobifunctional compound of 2-((1-((4-(6-((6-acetyl-8-cyclopentyl-5-methyl-7-oxo-7,8-dihydropyrido[2,3-d]pyrimidin-2-yl)amino)pyridin-3-yl)cyclohexyl)glycyl)piperidin-4-yl)amino)-N-(6-(dimethylamino)pyridazin-3-yl)-6-(isopropylamino)nicotinamide, or a pharmaceutically acceptable salt thereof.
F390. A heterobifunctional compound of 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)piperidin-4-yl)amino)-N-(6-(dimethylamino)pyridazin-3-yl)-6-(isopropylamino)nicotinamide, or a pharmaceutically acceptable salt thereof.
F391. A heterobifunctional compound of 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F392. A heterobifunctional compound of 2-((1-(2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetyl)piperidin-4-yl)amino)-N-(6-(dimethylamino)pyridazin-3-yl)-6-(isopropylamino)nicotinamide, or a pharmaceutically acceptable salt thereof.
F393. A heterobifunctional compound of 2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxamido)spiro[3.3]heptan-2-yl)amino)-N-(6-(dimethylamino)pyridazin-3-yl)-6-(isopropylamino)nicotinamide, or a pharmaceutically acceptable salt thereof.
F394. A heterobifunctional compound of 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)azetidin-3-yl)amino)-N-(6-(dimethylamino)pyridazin-3-yl)-6-(isopropylamino)nicotinamide, or a pharmaceutically acceptable salt thereof.
F395. A heterobifunctional compound of 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)pyrrolidin-3-yl)amino)-N-(6-(dimethylamino)pyridazin-3-yl)-6-(isopropylamino)nicotinamide, or a pharmaceutically acceptable salt thereof.
F396. A heterobifunctional compound of 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)piperidin-4-yl)amino)-N-(6-(dimethylamino)pyridazin-3-yl)-6-(isopropylamino)nicotinamide, or a pharmaceutically acceptable salt thereof.
F397. A heterobifunctional compound of 2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)spiro[3.3]heptan-2-yl)amino)-N-(6-(dimethylamino)pyridazin-3-yl)-6-(isopropylamino)nicotinamide, or a pharmaceutically acceptable salt thereof.
F398. A heterobifunctional compound of 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F399. A heterobifunctional compound of 2-((−4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide, or a pharmaceutically acceptable salt thereof.
F400. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)spiro[3.3]heptan-2-yl)amino)-N-(6-(dimethylamino)pyridazin-3-yl)-6-(isopropylamino)nicotinamide, or a pharmaceutically acceptable salt thereof.
F401. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)spiro[3.3]heptan-2-yl)amino)-N-(6-(dimethylamino)pyridazin-3-yl)-6-(isopropylamino)nicotinamide, or a pharmaceutically acceptable salt thereof.
F402. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-(isopropylamino)nicotinamide, or a pharmaceutically acceptable salt thereof.
F403. A heterobifunctional compound of 2-(((1R,3S)-3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclopentyl)amino)-N-(6-(dimethylamino)pyridazin-3-yl)-6-(isopropylamino)nicotinamide, or a pharmaceutically acceptable salt thereof.
F404. A heterobifunctional compound of 2-((2-(3-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)propanoyl)-2-azaspiro[3.3]heptan-6-yl)amino)-N-(1,5-dimethyl-1H-pyrazol-3-yl)-6-(isopropylamino)nicotinamide, or a pharmaceutically acceptable salt thereof.
F405. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(2-cyclopropyl-1-methyl-1H-imidazol-4-yl)-6-((methyl-d3)amino)nicotinamide, or a pharmaceutically acceptable salt thereof.
F406. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(6-(dimethylamino)pyridazin-3-yl)-6-(isopropylamino)nicotinamide, or a pharmaceutically acceptable salt thereof.
F407. A heterobifunctional compound of 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(6-(dimethylamino)pyridazin-3-yl)-6-(isopropylamino)nicotinamide, or a pharmaceutically acceptable salt thereof.
F408. A heterobifunctional compound of 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(1,5-dimethyl-1H-pyrazol-3-yl)-6-(isopropylamino)nicotinamide, or a pharmaceutically acceptable salt thereof.
F409. A heterobifunctional compound of 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(2-cyclopropyl-1-methyl-1H-imidazol-4-yl)-6-(isopropylamino)nicotinamide, or a pharmaceutically acceptable salt thereof.
F410. A heterobifunctional compound of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propan-2-yl)cyclobutyl)amino)-6-(isopropylamino)-N-(5-methylisoxazol-3-yl)nicotinamide, or a pharmaceutically acceptable salt thereof.
F411. A heterobifunctional compound of 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane- 1-carbonyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F412. A heterobifunctional compound of 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F413. A heterobifunctional compound of 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(isopropylamino)pyridin-2-yl)amino)cyclobutyl)-6-azaspiro[2.5]octane-1-carboxamide, or a pharmaceutically acceptable salt thereof.
F414. A heterobifunctional compound o6-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(isopropylamino)pyridin-2-yl)amino)spiro[3.3]heptan-2-yl)-6-azaspiro[2.5]octane-1-carboxamide, or a pharmaceutically acceptable salt thereof.
F415. A heterobifunctional compound of 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(1,5-dimethyl-1H-pyrazol-3-yl)-6-(isopropylamino)nicotinamide, or a pharmaceutically acceptable salt thereof.
F416. A heterobifunctional compound of 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.4]octan-6-yl)amino)-N-(6-(dimethylamino)pyridazin-3-yl)-6-(methylamino)nicotinamide, or a pharmaceutically acceptable salt thereof.
F417. A heterobifunctional compound of 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(isopropylamino)pyridin-2-yl)amino)cyclopentyl)-6-azaspiro[2.5]octane-1-carboxamide, or a pharmaceutically acceptable salt thereof.
F418. A heterobifunctional compound of 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)piperidin-4-yl)amino)-N-(6-(dimethylamino)pyridazin-3-yl)-6-(isopropylamino)nicotinamide, or a pharmaceutically acceptable salt thereof.
F419. A heterobifunctional compound of 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-6-(isopropylamino)-N-(5-methylisoxazol-3-yl)nicotinamide, or a pharmaceutically acceptable salt thereof.
F420. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(2-cyclopropyl-1-methyl-1H-imidazol-4-yl)-6-(isopropylamino)nicotinamide, or a pharmaceutically acceptable salt thereof.
F421. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(1,5-dimethyl-1H-pyrazol-3-yl)-6-(isopropylamino)nicotinamide, or a pharmaceutically acceptable salt thereof.
F422. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F423. A heterobifunctional compound of 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F424. A heterobifunctional compound of 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)pyrrolidin-3-yl)amino)-N-(6-(dimethylamino)pyridazin-3-yl)-6-(isopropylamino)nicotinamide, or a pharmaceutically acceptable salt thereof.
F425. A heterobifunctional compound of 2-((2-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F426. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)pyrrolidin-3-yl)amino)-N-(6-(dimethylamino)pyridazin-3-yl)-6-(isopropylamino)nicotinamide, or a pharmaceutically acceptable salt thereof.
F427. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)azetidin-3-yl)amino)-N-(6-(dimethylamino)pyridazin-3-yl)-6-(isopropylamino)nicotinamide, or a pharmaceutically acceptable salt thereof.
F428. A heterobifunctional compound of 2-((3-((8-(6-((6-acetyl-8-cyclopentyl-5-methyl-7-oxo-7,8-dihydropyrido[2,3-d]pyrimidin-2-yl)amino)pyridin-3-yl)-8-azaspiro[4.5]decan-2-yl)carbamoyl)cyclobutyl)amino)-N-(6-(dimethylamino)pyridazin-3-yl)-6-(methylamino)nicotinamide, or a pharmaceutically acceptable salt thereof.
F429. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F430. A heterobifunctional compound of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-N-(6-(dimethylamino)pyridazin-3-yl)-6-(methylamino)nicotinamide, or a pharmaceutically acceptable salt thereof.
F431. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)piperidin-4-yl)amino)-N-(2-cyclopropyl-1-methyl-1H-imidazol-4-yl)-6-(isopropylamino)nicotinamide, or a pharmaceutically acceptable salt thereof.
F432. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F433. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(1,5-dimethyl-1H-pyrazol-3-yl)-6-(isopropylamino)nicotinamide, or a pharmaceutically acceptable salt thereof.
F434. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(1,5-dimethyl-1H-pyrazol-3-yl)-6-((methyl-d3)amino)nicotinamide, or a pharmaceutically acceptable salt thereof.
F435. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(2-cyclopropyl-1-methyl-1H-imidazol-4-yl)-6-((methyl-d3)amino)nicotinamide, or a pharmaceutically acceptable salt thereof.
F436. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F437. A heterobifunctional compound of 7-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(isopropylamino)pyridin-2-yl)amino)spiro[3.3]heptan-2-yl)-7-azaspiro[3.5]nonane-2-carboxamide, or a pharmaceutically acceptable salt thereof.
F438. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(6-(dimethylamino)pyridazin-3-yl)-6-((methyl-d3)amino)nicotinamide, or a pharmaceutically acceptable salt thereof.
F439. A heterobifunctional compound of 6-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-((methyl-d3)amino)pyridin-2-yl)amino)spiro[3.3]heptan-2-yl)-6-azaspiro[2.5]octane-1-carboxamide, or a pharmaceutically acceptable salt thereof.
F440. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5,6-dihydro-4H-pyrrolo[1,2-b]pyrazol-2-yl)-6-(isopropylamino)nicotinamide, or a pharmaceutically acceptable salt thereof.
F441. A heterobifunctional compound of 2-((1-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)piperidin-4-yl)amino)-N-(6-(dimethylamino)pyridazin-3-yl)-6-((methyl-d3)amino)nicotinamide, or a pharmaceutically acceptable salt thereof.
F442. A heterobifunctional compound of 2-((1-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)azetidin-3-yl)amino)-N-(6-(dimethylamino)pyridazin-3-yl)-6-((methyl-d3)amino)nicotinamide, or a pharmaceutically acceptable salt thereof.
F443. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(2-cyclopropyl-1-methyl-1H-imidazol-4-yl)-6-((methyl-d3)amino)nicotinamide, or a pharmaceutically acceptable salt thereof.
F444. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(2-cyclopropyl-1-methyl-1H-imidazol-4-yl)-6-(isopropylamino)nicotinamide, or a pharmaceutically acceptable salt thereof.
F445. A heterobifunctional compound of 2-((3-((2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)methyl)cyclobutyl)amino)-N-(6-(dimethylamino)pyridazin-3-yl)-6-((methyl-d3)amino)nicotinamide, or a pharmaceutically acceptable salt thereof.
F446. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(1,5-dimethyl-1H-pyrazol-3-yl)-6-(isopropylamino)nicotinamide, or a pharmaceutically acceptable salt thereof.
F447. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(1,5-dimethyl-1H-pyrazol-3-yl)-6-((methyl-d3)amino)nicotinamide, or a pharmaceutically acceptable salt thereof.
F448. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F449. A heterobifunctional compound of 2-((6-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)spiro[3.3]heptan-2-yl)amino)-N-(6-(dimethylamino)pyridazin-3-yl)-6-((methyl-d3)amino)nicotinamide, or a pharmaceutically acceptable salt thereof.
F450. A heterobifunctional compound of 2-((3-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)cyclobutyl)amino)-N-(6-(dimethylamino)pyridazin-3-yl)-6-((methyl-d3)amino)nicotinamide, or a pharmaceutically acceptable salt thereof.
F451. A heterobifunctional compound of 6-(6-((6-acetyl-8-cyclopentyl-5-methyl-7-oxo-7,8-dihydropyrido[2,3-d]pyrimidin-2-yl)amino)pyridin-3-yl)-N-(6-((3-((1,5-dimethyl-1H-pyrazol-3-yl)carbamoyl)-6-(isopropylamino)pyridin-2-yl)amino)spiro[3.3]heptan-2-yl)-6-azaspiro[2.5]octane-1-carboxamide, or a pharmaceutically acceptable salt thereof.
F452. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-cyclopropyl-5-methyl-1H-pyrazol-3-yl)-6-(isopropylamino)nicotinamide, or a pharmaceutically acceptable salt thereof.
F453. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(6-methoxypyridin-2-yl)nicotinamide, or a pharmaceutically acceptable salt thereof.
F454. A heterobifunctional compound of 2-((1-(3-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)propanoyl)piperidin-4-yl)amino)-N-(6-(dimethylamino)pyridazin-3-yl)-6-(isopropylamino)nicotinamide, or a pharmaceutically acceptable salt thereof.
F455. A heterobifunctional compound of 2-((2-(3-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)propanoyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F456. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F457. A heterobifunctional compound of 6-(6-((6-acetyl-8-cyclopentyl-5-methyl-7-oxo-7,8-dihydropyrido[2,3-d]pyrimidin-2-yl)amino)pyridin-3-yl)-N-(6-((3-((2-cyclopropyl-1-methyl-1H-imidazol-4-yl)carbamoyl)-6-(isopropylamino)pyridin-2-yl)amino)spiro[3.3]heptan-2-yl)-6-azaspiro[2.5]octane-1-carboxamide, or a pharmaceutically acceptable salt thereof.
F458. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F459. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F460. A heterobifunctional compound of 2-((3-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)cyclobutyl)amino)-N-(6-(dimethylamino)pyridazin-3-yl)-6-(isopropylamino)nicotinamide, or a pharmaceutically acceptable salt thereof.
F461. A heterobifunctional compound of 2-((3-((1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)methyl)cyclobutyl)amino)-N-(6-(dimethylamino)pyridazin-3-yl)-6-(isopropylamino)nicotinamide, or a pharmaceutically acceptable salt thereof.
F462. A heterobifunctional compound of 2-((4-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)cyclohexyl)amino)-N-(6-(dimethylamino)pyridazin-3-yl)-6-(isopropylamino)nicotinamide, or a pharmaceutically acceptable salt thereof.
F463. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(6-(dimethylamino)pyridazin-3-yl)-6-((methyl-d3)amino)nicotinamide, or a pharmaceutically acceptable salt thereof.
F464. A heterobifunctional compound of 2-((2-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F465. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(1,5-dimethyl-1H-pyrazol-3-yl)-6-(isopropylamino)nicotinamide, or a pharmaceutically acceptable salt thereof.
F466. A heterobifunctional compound of 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(2-cyclopropyl-1-methyl-1H-imidazol-4-yl)-6-(isopropylamino)nicotinamide, or a pharmaceutically acceptable salt thereof.
F467. A heterobifunctional compound of 2-((6-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F468. A heterobifunctional compound of 2-((3-((2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetamido)methyl)cyclobutyl)amino)-N-(6-(dimethylamino)pyridazin-3-yl)-6-(isopropylamino)nicotinamide, or a pharmaceutically acceptable salt thereof.
F469. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)piperidin-4-yl)amino)-N-(6-(dimethylamino)pyridazin-3-yl)-6-(isopropylamino)nicotinamide, or a pharmaceutically acceptable salt thereof.
F470. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)spiro[3.3]heptan-2-yl)amino)-N-(6-(dimethylamino)pyridazin-3-yl)-6-(isopropylamino)nicotinamide, or a pharmaceutically acceptable salt thereof.
F471. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-((methyl-d3)amino)nicotinamide, or a pharmaceutically acceptable salt thereof.
F472. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F473. A heterobifunctional compound of 2-((4-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclohexyl)amino)-N-(6-(dimethylamino)pyridazin-3-yl)-6-(isopropylamino)nicotinamide, or a pharmaceutically acceptable salt thereof.
F474. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-((methyl-d3)amino)nicotinamide, or a pharmaceutically acceptable salt thereof.
F475. A heterobifunctional compound of 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclobutyl)amino)-N-(6-(dimethylamino)pyridazin-3-yl)-6-(isopropylamino)nicotinamide, or a pharmaceutically acceptable salt thereof.
F476. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-((methyl-d3)amino)nicotinamide, or a pharmaceutically acceptable salt thereof.
F477. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-((methyl-d3)amino)nicotinamide, or a pharmaceutically acceptable salt thereof.
F478. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-(isopropylamino)nicotinamide, or a pharmaceutically acceptable salt thereof.
F479. A heterobifunctional compound of 2-((3-((2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)methyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F480. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(2-cyclopropyl-1-methyl-1H-imidazol-4-yl)-6-((methyl-d3)amino)nicotinamide, or a pharmaceutically acceptable salt thereof.
F481. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(2-cyclopropyl-1-methyl-1H-imidazol-4-yl)-6-(isopropylamino)nicotinamide, or a pharmaceutically acceptable salt thereof.
F482. A heterobifunctional compound of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)piperidin-4-yl)amino)-N-(6-(dimethylamino)pyridazin-3-yl)-6-((methyl-d3)amino)nicotinamide, or a pharmaceutically acceptable salt thereof.
F483. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)cyclohexyl)amino)-N-(6-(dimethylamino)pyridazin-3-yl)-6-(isopropylamino)nicotinamide, or a pharmaceutically acceptable salt thereof.
F484. A heterobifunctional compound of 2-((1-(N-(2-(6-((6-acetyl-8-cyclopentyl-5-methyl-7-oxo-7,8-dihydropyrido[2,3-d]pyrimidin-2-yl)amino)pyridin-3-yl)-2-azaspiro[3.3]heptan-6-yl)-N-methylglycyl)piperidin-4-yl)amino)-N-(6-(dimethylamino)pyridazin-3-yl)-6-(isopropylamino)nicotinamide, or a pharmaceutically acceptable salt thereof.
F485. A heterobifunctional compound of 2-((3-((2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)methyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F486. A heterobifunctional compound of 2-((6-((1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)carbamoyl)spiro[3.3]heptan-2-yl)amino)-N-(6-(dimethylamino)pyridazin-3-yl)-6-(isopropylamino)nicotinamide, or a pharmaceutically acceptable salt thereof.
F487. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-(isopropylamino)nicotinamide, or a pharmaceutically acceptable salt thereof.
F488. A heterobifunctional compound of 2-((2-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F489. A heterobifunctional compound of 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F490. A heterobifunctional compound of 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)piperidin-4-yl)amino)-N-(6-(dimethylamino)pyridazin-3-yl)-6-((methyl-d3)amino)nicotinamide, or a pharmaceutically acceptable salt thereof.
F491. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)piperidin-4-yl)amino)-N-(6-(dimethylamino)pyridazin-3-yl)-6-((methyl-d3)amino)nicotinamide, or a pharmaceutically acceptable salt thereof.
F492. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-ethylnicotinamide, or a pharmaceutically acceptable salt thereof.
F493. A heterobifunctional compound of 2-((3-((2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)methyl)cyclobutyl)amino)-N-(6-(dimethylamino)pyridazin-3-yl)-6-(isopropylamino)nicotinamide, or a pharmaceutically acceptable salt thereof.
F494. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)cyclobutyl)amino)-N-(6-(dimethylamino)pyridazin-3-yl)-6-((methyl-d3)amino)nicotinamide, or a pharmaceutically acceptable salt thereof.
F495. A heterobifunctional compound of 2-((4-(4-((6-((6-acetyl-8-cyclopentyl-5-methyl-7-oxo-7,8-dihydropyrido[2,3-d]pyrimidin-2-yl)amino)pyridin-3-yl)methyl)piperazine-1-carbonyl)cyclohexyl)amino)-N-(6-(dimethylamino)pyridazin-3-yl)-6-((methyl-d3)amino)nicotinamide, or a pharmaceutically acceptable salt thereof.
F496. A heterobifunctional compound of 2-((3-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)bicyclo[1.1.1]pentan-1-yl)amino)-N-(6-(dimethylamino)pyridazin-3-yl)-6-(isopropylamino)nicotinamide, or a pharmaceutically acceptable salt thereof.
F497. A heterobifunctional compound of 2-((2-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)-2-oxoethyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F498. A heterobifunctional compound of 2-((1-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)-2-oxoethyl)piperidin-4-yl)amino)-N-(6-(dimethylamino)pyridazin-3-yl)-6-(isopropylamino)nicotinamide, or a pharmaceutically acceptable salt thereof.
F499. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-isopropylnicotinamide, or a pharmaceutically acceptable salt thereof.
F500. A heterobifunctional compound of 2-((trans-4-(3-(4-(6-((6-acetyl-8-cyclopentyl-5-ethyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-(isopropylamino)-N-(6-methoxypyridazin-3-yl)nicotinamide, or a pharmaceutically acceptable salt thereof.
F501. A heterobifunctional compound of 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclopentyl)amino)-N-(6-(dimethylamino)pyridazin-3-yl)-6-(isopropylamino)nicotinamide, or a pharmaceutically acceptable salt thereof.
F502. A heterobifunctional compound of 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)bicyclo[1.1.1]pentan-1-yl)amino)-N-(6-(dimethylamino)pyridazin-3-yl)-6-(isopropylamino)nicotinamide, or a pharmaceutically acceptable salt thereof.
F503. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)bicyclo[1.1.1]pentan-1-yl)amino)-N-(6-(dimethylamino)pyridazin-3-yl)-6-(isopropylamino)nicotinamide, or a pharmaceutically acceptable salt thereof.
F504. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanamido)bicyclo[1.1.1]pentan-1-yl)amino)-N-(6-(dimethylamino)pyridazin-3-yl)-6-(isopropylamino)nicotinamide, or a pharmaceutically acceptable salt thereof.
F505. A heterobifunctional compound of 2-((3-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)acetamido)bicyclo[1.1.1]pentan-1-yl)amino)-N-(6-(dimethylamino)pyridazin-3-yl)-6-(isopropylamino)nicotinamide, or a pharmaceutically acceptable salt thereof.
F506. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamoyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F507. A heterobifunctional compound of 2-((2-(2-(7-((6-((6-acetyl-8-cyclopentyl-5-methyl-7-oxo-7,8-dihydropyrido[2,3-d]pyrimidin-2-yl)amino)pyridin-3-yl)methyl)-2,7-diazaspiro[4.4]nonan-2-yl)-2-oxoethyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F508. A heterobifunctional compound of 2-((2-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)acetyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F509. A heterobifunctional compound of 2-((1-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)acetyl)piperidin-4-yl)amino)-N-(6-(dimethylamino)pyridazin-3-yl)-6-(isopropylamino)nicotinamide, or a pharmaceutically acceptable salt thereof.
F510. A heterobifunctional compound of 2-((3-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclobutyl)amino)-N-(6-(dimethylamino)pyridazin-3-yl)-6-(isopropylamino)nicotinamide, or a pharmaceutically acceptable salt thereof.
F511. A heterobifunctional compound of 2-((3-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclopentyl)amino)-N-(6-(dimethylamino)pyridazin-3-yl)-6-(isopropylamino)nicotinamide, or a pharmaceutically acceptable salt thereof.
F512. A heterobifunctional compound of 2-((4-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclohexyl)amino)-N-(6-(dimethylamino)pyridazin-3-yl)-6-(isopropylamino)nicotinamide, or a pharmaceutically acceptable salt thereof.
F5 13. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)azetidin-3-yl)amino)-N-(6-(dimethylamino)pyridazin-3-yl)-6-(methylamino)nicotinamide, or a pharmaceutically acceptable salt thereof.
F514. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(5-methyl-1-(trifluoromethyl)-1H-pyrazol-3-yl)nicotinamide, or a pharmaceutically acceptable salt thereof.
F515. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.4]octan-6-yl)amino)-N-(6-(dimethylamino)pyridazin-3-yl)-6-(isopropylamino)nicotinamide, or a pharmaceutically acceptable salt thereof.
F516. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-methylcyclohexyl)amino)-N-(6-(dimethylamino)pyridazin-3-yl)-6-(isopropylamino)nicotinamide, or a pharmaceutically acceptable salt thereof.
F517. A heterobifunctional compound of 2-((1-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)-2-oxoethyl)azetidin-3-yl)amino)-N-(6-(dimethylamino)pyridazin-3-yl)-6-(isopropylamino)nicotinamide, or a pharmaceutically acceptable salt thereof.
F518. A heterobifunctional compound of 2-((2-(2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidin-1-yl)-2-oxoethyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F519. A heterobifunctional compound of 2-((2-(2-(7-((6-((6-acetyl-8-cyclopentyl-5-methyl-7-oxo-7,8-dihydropyrido[2,3-d]pyrimidin-2-yl)amino)pyridin-3-yl)methyl)-2,7-diazaspiro[4.4]nonan-2-yl)acetyl)-2-azaspiro[3.3]heptan-6-yl)amino)-N-(6-(dimethylamino)pyridazin-3-yl)-6-((methyl-d3)amino)nicotinamide, or a pharmaceutically acceptable salt thereof.
F520. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(5,6-dihydro-4H-pyrrolo[1,2-b]pyrazol-2-yl)-6-(isopropylamino)nicotinamide, or a pharmaceutically acceptable salt thereof.
F521. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(5,6-dihydro-4H-pyrrolo[1,2-b]pyrazol-2-yl)-6-((methyl-d3)amino)nicotinamide, or a pharmaceutically acceptable salt thereof.
F522. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-(isopropylamino)-N-(6-methoxypyridin-2-yl)nicotinamide, or a pharmaceutically acceptable salt thereof.
F523. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-methoxypyridin-2-yl)-6-((methyl-d3)amino)nicotinamide, or a pharmaceutically acceptable salt thereof.
F524. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(5,6-dihydro-4H-pyrrolo[1,2-b]pyrazol-2-yl)-6-((methyl-d3)amino)nicotinamide, or a pharmaceutically acceptable salt thereof.
F525. A heterobifunctional compound of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)methyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F526. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-ethylbenzamide, or a pharmaceutically acceptable salt thereof.
F527. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-(isopropylamino)benzamide, or a pharmaceutically acceptable salt thereof.
F528. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-isopropylbenzamide, or a pharmaceutically acceptable salt thereof.
F529. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(5,6-dihydro-4H-pyrrolo[1,2-b]pyrazol-2-yl)-6-(isopropylamino)nicotinamide, or a pharmaceutically acceptable salt thereof.
F530. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(5,6-dihydro-4H-pyrrolo[1,2-b]pyrazol-2-yl)-6-(isopropylamino)nicotinamide, or a pharmaceutically acceptable salt thereof.
F531. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-methoxypyridin-2-yl)-6-((methyl-d3)amino)nicotinamide, or a pharmaceutically acceptable salt thereof.
F532. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-6-(isopropylamino)-N-(6-methoxypyridin-2-yl)nicotinamide, or a pharmaceutically acceptable salt thereof.
F533. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-6-(isopropylamino)-N-(6-methoxypyridin-2-yl)nicotinamide, or a pharmaceutically acceptable salt thereof.
F534. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F535. A heterobifunctional compound of 2-((4-(3-(4-(6-((8-cyclopentyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide, or a pharmaceutically acceptable salt thereof.
F536. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-methoxycyclohexyl)amino)-N-(6-(dimethylamino)pyridazin-3-yl)-6-(isopropylamino)nicotinamide, or a pharmaceutically acceptable salt thereof.
F537. A heterobifunctional compound of 2-((3-(2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidin-1-yl)-2-oxoethyl)bicyclo[1.1.1]pentan-1-yl)amino)-N-(6-(dimethylamino)pyridazin-3-yl)-6-(isopropylamino)nicotinamide, or a pharmaceutically acceptable salt thereof.
F538. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F539. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-6-azaspiro[3.4]octan-2-yl)amino)-N-(6-(dimethylamino)pyridazin-3-yl)-6-(isopropylamino)nicotinamide, or a pharmaceutically acceptable salt thereof.
F540. A heterobifunctional compound of trans-6-acetyl-8-cyclopentyl-2-((5-(4-(1-(4-((3-(5-(dimethylamino)-1H-benzo[d]imidazol-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one, or a pharmaceutically acceptable salt thereof.
F541. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)piperidin-4-yl)amino)-N-(6-(dimethylamino)pyridazin-3-yl)-6-(isopropylamino)nicotinamide, or a pharmaceutically acceptable salt thereof.
F542. A heterobifunctional compound of (S)-2-((3-((1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-2-carboxamido)methyl)cyclobutyl)amino)-N-(6-(dimethylamino)pyridazin-3-yl)-6-(isopropylamino)nicotinamide, or a pharmaceutically acceptable salt thereof.
F543. A heterobifunctional compound of (S)-2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-2-carboxamido)spiro[3.3]heptan-2-yl)amino)-N-(6-(dimethylamino)pyridazin-3-yl)-6-(isopropylamino)nicotinamide, or a pharmaceutically acceptable salt thereof.
F544. A heterobifunctional compound of 2-((2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)-L-prolyl)-2-azaspiro[3.3]heptan-6-yl)amino)-N-(6-(dimethylamino)pyridazin-3-yl)-6-(isopropylamino)nicotinamide, or a pharmaceutically acceptable salt thereof.
F545. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[3.4]octan-6-yl)amino)-N-(6-(dimethylamino)pyridazin-3-yl)-6-(isopropylamino)nicotinamide, or a pharmaceutically acceptable salt thereof.
F546. A heterobifunctional compound of trans-6-acetyl-8-cyclopentyl-2-((5-(4-(1-(4-((3-(5-(dimethylamino)-3H-imidazo[4,5-b]pyridin-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one, or a pharmaceutically acceptable salt thereof.
F547. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(1,5-dimethyl-1H-pyrazol-3-yl)-6-(isopropylamino)nicotinamide, or a pharmaceutically acceptable salt thereof.
F548. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(2-cyclopropyl-1-methyl-1H-imidazol-4-yl)-6-(isopropylamino)nicotinamide, or a pharmaceutically acceptable salt thereof.
F549. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(5,6-dihydro-4H-pyrrolo[1,2-b]pyrazol-2-yl)-6-(isopropylamino)nicotinamide, or a pharmaceutically acceptable salt thereof.
F550. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanoyl)-2-azaspiro[4.4]nonan-7-yl)amino)-N-(6-(dimethylamino)pyridazin-3-yl)-6-(isopropylamino)nicotinamide, or a pharmaceutically acceptable salt thereof.
F551. A heterobifunctional compound of 2-((2-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetyl)-2-azaspiro[4.4]nonan-7-yl)amino)-N-(6-(dimethylamino)pyridazin-3-yl)-6-(isopropylamino)nicotinamide, or a pharmaceutically acceptable salt thereof.
F552. A heterobifunctional compound of trans-6-acetyl-8-cyclopentyl-2-((5-(4-(1-(trans-4-((3-(6-(dimethylamino)-3H-imidazo[4,5-c]pyridin-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one, or a pharmaceutically acceptable salt thereof.
F553. A heterobifunctional compound of N-(6-(dimethylamino)pyridazin-3-yl)-2-((2-(2-(4-((6-((4-(2-ethyl-1-oxo-1,2,3,4-tetrahydroisoquinolin-6-yl)-5-fluoropyrimidin-2-yl)amino)pyridin-3-yl)methyl)piperazin-1-yl)acetyl)-2-azaspiro[3.3]heptan-6-yl)amino)-6-(isopropylamino)nicotinamide, or a pharmaceutically acceptable salt thereof.
F554. A heterobifunctional compound of N-(6-(dimethylamino)pyridazin-3-yl)-2-((6-(2-(4-((6-((4-(2-ethyl-1-oxo-1,2,3,4-tetrahydroisoquinolin-6-yl)-5-fluoropyrimidin-2-yl)amino)pyridin-3-yl)methyl)piperazin-1-yl)acetamido)spiro[3.3]heptan-2-yl)amino)-6-(isopropylamino)nicotinamide, or a pharmaceutically acceptable salt thereof.
F555. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[3.5]nonan-7-yl)amino)-N-(6-(dimethylamino)pyridazin-3-yl)-6-(isopropylamino)nicotinamide, or a pharmaceutically acceptable salt thereof.
F556. A heterobifunctional compound of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)spiro[3.4]octan-2-yl)amino)-N-(6-(dimethylamino)pyridazin-3-yl)-6-(isopropylamino)nicotinamide, or a pharmaceutically acceptable salt thereof.
F557. A heterobifunctional compound of N-(6-(dimethylamino)pyridazin-3-yl)-6-(isopropylamino)-2-((2-(2-(4-((6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)methyl)piperazin-1-yl)acetyl)-2-azaspiro[3.3]heptan-6-yl)amino)nicotinamide, or a pharmaceutically acceptable salt thereof.
F558. A heterobifunctional compound of N-(6-(dimethylamino)pyridazin-3-yl)-6-(isopropylamino)-2-((6-(2-(4-((6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)methyl)piperazin-1-yl)acetamido)spiro[3.3]heptan-2-yl)amino)nicotinamide, or a pharmaceutically acceptable salt thereof.
F559. A heterobifunctional compound of 2-((trans-4-(3-(4-(6-((8-cyclopentyl-6-(difluoromethyl)-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide, or a pharmaceutically acceptable salt thereof.
F560. A heterobifunctional compound of 2-((trans-4-(3-(4-(6-((6-acetyl-8-cyclopentyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide, or a pharmaceutically acceptable salt thereof.
F561. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[4.4]nonan-7-yl)amino)-N-(6-(dimethylamino)pyridazin-3-yl)-6-(isopropylamino)nicotinamide, or a pharmaceutically acceptable salt thereof.
F562. A heterobifunctional compound of 7-cyclopentyl-2-((5-((4-(2-((6-((3-((6-(dimethylamino)pyridazin-3-yl)carbamoyl)-6-(isopropylamino)pyridin-2-yl)amino)spiro[3.3]heptan-2-yl)amino)-2-oxoethyl)piperazin-1-yl)methyl)pyridin-2-yl)amino)-N,N-dimethyl-7H-pyrrolo[2,3-d]pyrimidine-6-carboxamide, or a pharmaceutically acceptable salt thereof.
F563. A heterobifunctional compound of 7-cyclopentyl-2-((5-((4-(2-(6-((3-((6-(dimethylamino)pyridazin-3-yl)carbamoyl)-6-(isopropylamino)pyridin-2-yl)amino)-2-azaspiro[3.3]heptan-2-yl)-2-oxoethyl)piperazin-1-yl)methyl)pyridin-2-yl)amino)-N,N-dimethyl-7H-pyrrolo[2,3-d]pyrimidine-6-carboxamide, or a pharmaceutically acceptable salt thereof.
F564. A heterobifunctional compound of 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6,7-dihydro-5H-pyrrolo[1,2-a]imidazol-2-yl)-6-(isopropylamino)nicotinamide, or a pharmaceutically acceptable salt thereof.
F565. A heterobifunctional compound of 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6,7-dihydro-5H-pyrrolo[1,2-a]imidazol-2-yl)-6-(isopropylamino)nicotinamide, or a pharmaceutically acceptable salt thereof.
F566. A heterobifunctional compound of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6,7-dihydro-5H-pyrrolo[1,2-a]imidazol-2-yl)-6-(isopropylamino)nicotinamide, or a pharmaceutically acceptable salt thereof.
F567. A heterobifunctional compound of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6,7-dihydro-5H-pyrrolo[1,2-a]imidazol-2-yl)-6-((methyl-d3)amino)nicotinamide, or a pharmaceutically acceptable salt thereof.
F568. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6,7-dihydro-5H-pyrrolo[1,2-a]imidazol-2-yl)-6-(isopropylamino)nicotinamide, or a pharmaceutically acceptable salt thereof.
F569. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6,7-dihydro-5H-pyrrolo[1,2-a]imidazol-2-yl)-6-((methyl-d3)amino)nicotinamide, or a pharmaceutically acceptable salt thereof.
F570. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-6-azaspiro[3.4]octan-2-yl)amino)-N-(6-(dimethylamino)pyridazin-3-yl)-6-(isopropylamino)nicotinamide, or a pharmaceutically acceptable salt thereof.
F571. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(trifluoromethyl)nicotinamide, or a pharmaceutically acceptable salt thereof.
F572. A heterobifunctional compound of 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-fluorocyclohexyl)amino)-N-(6-(dimethylamino)pyridazin-3-yl)-6-(isopropylamino)nicotinamide, or a pharmaceutically acceptable salt thereof.
F573. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-cyclopropyl-N-(6-(dimethylamino)pyridazin-3-yl)nicotinamide, or a pharmaceutically acceptable salt thereof.
F574. A heterobifunctional compound of 2-((cis-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-hydroxycyclohexyl)amino)-N-(6-(dimethylamino)pyridazin-3-yl)-6-(isopropylamino)nicotinamide, or a pharmaceutically acceptable salt thereof.
F575. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-hydroxycyclohexyl)amino)-N-(6-(dimethylamino)pyridazin-3-yl)-6-(isopropylamino)nicotinamide, or a pharmaceutically acceptable salt thereof.
F576. A heterobifunctional compound of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-methylnicotinamide, or a pharmaceutically acceptable salt thereof.
F577. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-ethylnicotinamide, or a pharmaceutically acceptable salt thereof.
F578. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-ethylnicotinamide, or a pharmaceutically acceptable salt thereof.
F579. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-isopropylnicotinamide, or a pharmaceutically acceptable salt thereof.
F580. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-isopropylnicotinamide, or a pharmaceutically acceptable salt thereof.
F581. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-6-cyclopropyl-N-(6-(dimethylamino)pyridazin-3-yl)nicotinamide, or a pharmaceutically acceptable salt thereof.
F582. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-ethylnicotinamide, or a pharmaceutically acceptable salt thereof.
F583. A heterobifunctional compound of 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-isopropylnicotinamide, or a pharmaceutically acceptable salt thereof.
F584. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(5,6-dihydro-4H-pyrrolo[1,2-b]pyrazol-2-yl)-6-(isopropylamino)nicotinamide, or a pharmaceutically acceptable salt thereof.
F585. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanamido)spiro[3.3]heptan-2-yl)amino)-N-(6-(dimethylamino)pyridazin-3-yl)-6-(isopropylamino)nicotinamide, or a pharmaceutically acceptable salt thereof.
F586. A heterobifunctional compound of 2-((6-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F587. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(1,5-dimethyl-1H-pyrazol-3-yl)-6-(isopropylamino)nicotinamide, or a pharmaceutically acceptable salt thereof.
F588. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(2-cyclopropyl-1-methyl-1H-imidazol-4-yl)-6-(isopropylamino)nicotinamide, or a pharmaceutically acceptable salt thereof.
F589. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(2-cyclopropyl-1-methyl-1H-imidazol-4-yl)-6-(isopropylamino)nicotinamide, or a pharmaceutically acceptable salt thereof.
F590. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-methylnicotinamide, or a pharmaceutically acceptable salt thereof.
F591. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-6-(isopropylamino)-N-(5-methylisoxazol-3-yl)nicotinamide, or a pharmaceutically acceptable salt thereof.
F592. A heterobifunctional compound of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(5,6-dihydro-4H-pyrrolo[1,2-b]pyrazol-2-yl)-6-(isopropylamino)nicotinamide, or a pharmaceutically acceptable salt thereof.
F593. A heterobifunctional compound of 2-((6-(2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F594. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-N-methylacetamido)spiro[3.3]heptan-2-yl)amino)-N-(6-(dimethylamino)pyridazin-3-yl)-6-(isopropylamino)nicotinamide, or a pharmaceutically acceptable salt thereof.
F595. A heterobifunctional compound of 2-((6-((1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)(methyl)carbamoyl)spiro[3.3]heptan-2-yl)amino)-N-(6-(dimethylamino)pyridazin-3-yl)-6-(isopropylamino)nicotinamide, or a pharmaceutically acceptable salt thereof.
F596. A heterobifunctional compound of 2-((6-(2-(1-((6-((6-acetyl-8-cyclopentyl-5-methyl-7-oxo-7,8-dihydropyrido[2,3-d]pyrimidin-2-yl)amino)pyridin-3-yl)methyl)pyrrolidin-3-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F597. A heterobifunctional compound of 2-((trans-4-(3-(4-(4-((6-acetyl-8-cyclopentyl-5-methyl-7-oxo-7,8-dihydropyrido[2,3-d]pyrimidin-2-yl)amino)cyclohexyl)piperazin-1-yl)pyrrolidine-1-carbonyl)cyclohexyl)amino)-N-(6-(dimethylamino)pyridazin-3-yl)-6-ethylnicotinamide, or a pharmaceutically acceptable salt thereof.
F598. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-4-ethylbenzamide, or a pharmaceutically acceptable salt thereof.
F599. A heterobifunctional compound of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperidin-1-yl)acetamido)spiro[3.3]heptan-2-yl)amino)-N-(6-(dimethylamino)pyridazin-3-yl)-6-(isopropylamino)nicotinamide, or a pharmaceutically acceptable salt thereof.
F600. A heterobifunctional compound of N-(6-(dimethylamino)pyridazin-3-yl)-2-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropyl)amino)-6-(methylamino)nicotinamide, or a pharmaceutically acceptable salt thereof.
F601. A heterobifunctional compound of N-(6-(dimethylamino)pyridazin-3-yl)-2-((4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-4-oxobutyl)amino)-6-(methylamino)nicotinamide, or a pharmaceutically acceptable salt thereof.
F602. A heterobifunctional compound of N-(6-(dimethylamino)pyridazin-3-yl)-2-((5-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-5-oxopentyl)amino)-6-(methylamino)nicotinamide, or a pharmaceutically acceptable salt thereof.
F603. A heterobifunctional compound of N-(6-(dimethylamino)pyridazin-3-yl)-2-((6-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-6-oxohexyl)amino)-6-(methylamino)nicotinamide, or a pharmaceutically acceptable salt thereof.
F604. A heterobifunctional compound of N-(6-(dimethylamino)pyridazin-3-yl)-2-((7-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-7-oxoheptyl)amino)-6-(methylamino)nicotinamide, or a pharmaceutically acceptable salt thereof.
F605. A heterobifunctional compound of N-(6-(dimethylamino)pyridazin-3-yl)-2-((8-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-8-oxooctyl)amino)-6-(methylamino)nicotinamide, or a pharmaceutically acceptable salt thereof.
F606. A heterobifunctional compound of N-(6-(dimethylamino)pyridazin-3-yl)-2-((2-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropoxy)ethyl)amino)-6-(methylamino)nicotinamide, or a pharmaceutically acceptable salt thereof.
F607. A heterobifunctional compound of N-(6-(dimethylamino)pyridazin-3-yl)-2-((2-(2-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropoxy)ethoxy)ethyl)amino)-6-(methylamino)nicotinamide, or a pharmaceutically acceptable salt thereof.
F608. A heterobifunctional compound of N-(6-(dimethylamino)pyridazin-3-yl)-2-((2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethyl)amino)-6-(methylamino)nicotinamide, or a pharmaceutically acceptable salt thereof.
F609. A heterobifunctional compound of N-(6-(dimethylamino)pyridazin-3-yl)-2-((3-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)propyl)amino)-6-(methylamino)nicotinamide, or a pharmaceutically acceptable salt thereof.
F610. A heterobifunctional compound of N-(6-(dimethylamino)pyridazin-3-yl)-2-((4-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)butyl)amino)-6-(methylamino)nicotinamide, or a pharmaceutically acceptable salt thereof.
F611. A heterobifunctional compound of N-(6-(dimethylamino)pyridazin-3-yl)-2-((5-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)pentyl)amino)-6-(methylamino)nicotinamide, or a pharmaceutically acceptable salt thereof.
F612. A heterobifunctional compound of N-(6-(dimethylamino)pyridazin-3-yl)-2-((6-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)hexyl)amino)-6-(methylamino)nicotinamide, or a pharmaceutically acceptable salt thereof.
F613. A heterobifunctional compound of N-(6-(dimethylamino)pyridazin-3-yl)-2-((7-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)heptyl)amino)-6-(methylamino)nicotinamide, or a pharmaceutically acceptable salt thereof.
F614. A heterobifunctional compound of N-(6-(dimethylamino)pyridazin-3-yl)-2-((8-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)octyl)amino)-6-(methylamino)nicotinamide, or a pharmaceutically acceptable salt thereof.
F6 15. A heterobifunctional compound of N-(6-(dimethylamino)pyridazin-3-yl)-2-((2-(2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethoxy)ethyl)amino)-6-(methylamino)nicotinamide, or a pharmaceutically acceptable salt thereof.
F616. A heterobifunctional compound of 7-cyclopentyl-2-((5-(4-(3-((3-((6-(dimethylamino)pyridazin-3-yl)carbamoyl)-6-(methylamino)pyridin-2-yl)amino)propanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F617. A heterobifunctional compound of 7-cyclopentyl-2-((5-(4-(4-((3-((6-(dimethylamino)pyridazin-3-yl)carbamoyl)-6-(methylamino)pyridin-2-yl)amino)butanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F618. A heterobifunctional compound of 7-cyclopentyl-2-((5-(4-(5-((3-((6-(dimethylamino)pyridazin-3-yl)carbamoyl)-6-(methylamino)pyridin-2-yl)amino)pentanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F619. A heterobifunctional compound of 7-cyclopentyl-2-((5-(4-(6-((3-((6-(dimethylamino)pyridazin-3-yl)carbamoyl)-6-(methylamino)pyridin-2-yl)amino)hexanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F620. A heterobifunctional compound of 7-cyclopentyl-2-((5-(4-(7-((3-((6-(dimethylamino)pyridazin-3-yl)carbamoyl)-6-(methylamino)pyridin-2-yl)amino)heptanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F621. A heterobifunctional compound of 7-cyclopentyl-2-((5-(4-(8-((3-((6-(dimethylamino)pyridazin-3-yl)carbamoyl)-6-(methylamino)pyridin-2-yl)amino)octanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F622. A heterobifunctional compound of 7-cyclopentyl-2-((5-(4-(3-(2-((3-((6-(dimethylamino)pyridazin-3-yl)carbamoyl)-6-(methylamino)pyridin-2-yl)amino)ethoxy)propanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F623. A heterobifunctional compound of 7-cyclopentyl-2-((5-(4-(3-(2-(2-((3-((6-(dimethylamino)pyridazin-3-yl)carbamoyl)-6-(methylamino)pyridin-2-yl)amino)ethoxy)ethoxy)propanoyl)piperazin-1-yl)pyridin-2-yl)amino)-N,N-dimethyl-7H-pyrrolo[2,3-d]pyrimidine-6-carboxamide, or a pharmaceutically acceptable salt thereof.
F624. A heterobifunctional compound of 7-cyclopentyl-2-((5-(4-(2-((2-((3-((6-(dimethylamino)pyridazin-3-yl)carbamoyl)-6-(methylamino)pyridin-2-yl)amino)ethyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F625. A heterobifunctional compound of 7-cyclopentyl-2-((5-(4-(2-((3-((3-((6-(dimethylamino)pyridazin-3-yl)carbamoyl)-6-(methylamino)pyridin-2-yl)amino)propyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F626. A heterobifunctional compound of 7-cyclopentyl-2-((5-(4-(2-((4-((3-((6-(dimethylamino)pyridazin-3-yl)carbamoyl)-6-(methylamino)pyridin-2-yl)amino)butyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F627. A heterobifunctional compound of 7-cyclopentyl-2-((5-(4-(2-((5-((3-((6-(dimethylamino)pyridazin-3-yl)carbamoyl)-6-(methylamino)pyridin-2-yl)amino)pentyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F628. A heterobifunctional compound of 7-cyclopentyl-2-((5-(4-(2-((6-((3-((6-(dimethylamino)pyridazin-3-yl)carbamoyl)-6-(methylamino)pyridin-2-yl)amino)hexyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F629. A heterobifunctional compound of 7-cyclopentyl-2-((5-(4-(2-((7-((3-((6-(dimethylamino)pyridazin-3-yl)carbamoyl)-6-(methylamino)pyridin-2-yl)amino)heptyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F630. A heterobifunctional compound of 7-cyclopentyl-2-((5-(4-(2-((8-((3-((6-(dimethylamino)pyridazin-3-yl)carbamoyl)-6-(methylamino)pyridin-2-yl)amino)octyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F631. A heterobifunctional compound of 7-cyclopentyl-2-((5-(4-(2-((2-(2-((3-((6-(dimethylamino)pyridazin-3-yl)carbamoyl)-6-(methylamino)pyridin-2-yl)amino)ethoxy)ethyl)amino)-2-oxoethyl)piperazin-1-yl)pyridin-2-yl)amino)-N,N-dimethyl-7H-pyrrolo[2,3-d]pyrimidine-6-carboxamide, or a pharmaceutically acceptable salt thereof.
F632. A heterobifunctional compound of N-(6-(dimethylamino)pyridazin-3-yl)-6-(methylamino)-2-((3-oxo-3-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)propyl)amino)nicotinamide, or a pharmaceutically acceptable salt thereof.
F633. A heterobifunctional compound of N-(6-(dimethylamino)pyridazin-3-yl)-6-(methylamino)-2-((4-oxo-4-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)butyl)amino)nicotinamide, or a pharmaceutically acceptable salt thereof.
F634. A heterobifunctional compound of N-(6-(dimethylamino)pyridazin-3-yl)-6-(methylamino)-2-((5-oxo-5-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)pentyl)amino)nicotinamide, or a pharmaceutically acceptable salt thereof.
F635. A heterobifunctional compound of N-(6-(dimethylamino)pyridazin-3-yl)-6-(methylamino)-2-((6-oxo-6-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)hexyl)amino)nicotinamide, or a pharmaceutically acceptable salt thereof.
F636. A heterobifunctional compound of N-(6-(dimethylamino)pyridazin-3-yl)-6-(methylamino)-2-((7-oxo-7-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)heptyl)amino)nicotinamide, or a pharmaceutically acceptable salt thereof.
F637. A heterobifunctional compound of N-(6-(dimethylamino)pyridazin-3-yl)-6-(methylamino)-2-((8-oxo-8-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)octyl)amino)nicotinamide, or a pharmaceutically acceptable salt thereof.
F638. A heterobifunctional compound of N-(6-(dimethylamino)pyridazin-3-yl)-6-(methylamino)-2-((2-(3-oxo-3-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)propoxy)ethyl)amino)nicotinamide, or a pharmaceutically acceptable salt thereof.
F639. A heterobifunctional compound of N-(6-(dimethylamino)pyridazin-3-yl)-6-(methylamino)-2-((2-(2-(3-oxo-3-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)propoxy)ethoxy)ethyl)amino)nicotinamide, or a pharmaceutically acceptable salt thereof.
F640. A heterobifunctional compound of N-(6-(dimethylamino)pyridazin-3-yl)-6-(methylamino)-2-((2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethyl)amino)nicotinamide, or a pharmaceutically acceptable salt thereof.
F641. A heterobifunctional compound of N-(6-(dimethylamino)pyridazin-3-yl)-6-(methylamino)-2-((3-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)propyl)amino)nicotinamide, or a pharmaceutically acceptable salt thereof.
F642. A heterobifunctional compound of N-(6-(dimethylamino)pyridazin-3-yl)-6-(methylamino)-2-((4-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)butyl)amino)nicotinamide, or a pharmaceutically acceptable salt thereof.
F643. A heterobifunctional compound of N-(6-(dimethylamino)pyridazin-3-yl)-6-(methylamino)-2-((5-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)pentyl)amino)nicotinamide, or a pharmaceutically acceptable salt thereof.
F644. A heterobifunctional compound of N-(6-(dimethylamino)pyridazin-3-yl)-6-(methylamino)-2-((6-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)hexyl)amino)nicotinamide, or a pharmaceutically acceptable salt thereof.
F645. A heterobifunctional compound of N-(6-(dimethylamino)pyridazin-3-yl)-6-(methylamino)-2-((7-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)heptyl)amino)nicotinamide, or a pharmaceutically acceptable salt thereof.
F646. A heterobifunctional compound of N-(6-(dimethylamino)pyridazin-3-yl)-6-(methylamino)-2-((8-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)octyl)amino)nicotinamide, or a pharmaceutically acceptable salt thereof.
F647. A heterobifunctional compound of N-(6-(dimethylamino)pyridazin-3-yl)-6-(methylamino)-2-((2-(2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethoxy)ethyl)amino)nicotinamide, or a pharmaceutically acceptable salt thereof.
F648. A heterobifunctional compound of N-(6-(dimethylamino)pyridazin-3-yl)-2-((3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropyl)amino)-6-(methylamino)nicotinamide, or a pharmaceutically acceptable salt thereof.
F649. A heterobifunctional compound of N-(6-(dimethylamino)pyridazin-3-yl)-2-((4-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-4-oxobutyl)amino)-6-(methylamino)nicotinamide, or a pharmaceutically acceptable salt thereof.
F650. A heterobifunctional compound of N-(6-(dimethylamino)pyridazin-3-yl)-2-((5-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-5-oxopentyl)amino)-6-(methylamino)nicotinamide, or a pharmaceutically acceptable salt thereof.
F651. A heterobifunctional compound of N-(6-(dimethylamino)pyridazin-3-yl)-2-((6-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-6-oxohexyl)amino)-6-(methylamino)nicotinamide, or a pharmaceutically acceptable salt thereof.
F652. A heterobifunctional compound of N-(6-(dimethylamino)pyridazin-3-yl)-2-((7-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-7-oxoheptyl)amino)-6-(methylamino)nicotinamide, or a pharmaceutically acceptable salt thereof.
F653. A heterobifunctional compound of N-(6-(dimethylamino)pyridazin-3-yl)-2-((8-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-8-oxooctyl)amino)-6-(methylamino)nicotinamide, or a pharmaceutically acceptable salt thereof.
F654. A heterobifunctional compound of N-(6-(dimethylamino)pyridazin-3-yl)-2-((2-(3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropoxy)ethyl)amino)-6-(methylamino)nicotinamide, or a pharmaceutically acceptable salt thereof.
F655. A heterobifunctional compound of N-(6-(dimethylamino)pyridazin-3-yl)-2-((2-(2-(3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropoxy)ethoxy)ethyl)amino)-6-(methylamino)nicotinamide, or a pharmaceutically acceptable salt thereof.
F656. A heterobifunctional compound of N-(6-(dimethylamino)pyridazin-3-yl)-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)-6-(methylamino)nicotinamide, or a pharmaceutically acceptable salt thereof.
F657. A heterobifunctional compound of 7-cyclopentyl-2-((5-(4-((3-((6-(dimethylamino)pyridazin-3-yl)carbamoyl)-6-(methylamino)pyridin-2-yl)glycyl)piperazin-1-yl)pyridin-2-yl)amino)-N,N-dimethyl-7H-pyrrolo[2,3-d]pyrimidine-6-carboxamide, or a pharmaceutically acceptable salt thereof.
F658. A heterobifunctional compound of N-(6-(dimethylamino)pyridazin-3-yl)-2-((2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethyl)amino)-6-(methylamino)nicotinamide, or a pharmaceutically acceptable salt thereof.
F659. A heterobifunctional compound of N-(6-(dimethylamino)pyridazin-3-yl)-2-((3-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)propyl)amino)-6-(methylamino)nicotinamide, or a pharmaceutically acceptable salt thereof.
F660. A heterobifunctional compound of N-(6-(dimethylamino)pyridazin-3-yl)-2-((4-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)butyl)amino)-6-(methylamino)nicotinamide, or a pharmaceutically acceptable salt thereof.
F661. A heterobifunctional compound of N-(6-(dimethylamino)pyridazin-3-yl)-2-((5-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)pentyl)amino)-6-(methylamino)nicotinamide, or a pharmaceutically acceptable salt thereof.
F662. A heterobifunctional compound of N-(6-(dimethylamino)pyridazin-3-yl)-2-((6-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)hexyl)amino)-6-(methylamino)nicotinamide, or a pharmaceutically acceptable salt thereof.
F663. A heterobifunctional compound of N-(6-(dimethylamino)pyridazin-3-yl)-2-((7-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)heptyl)amino)-6-(methylamino)nicotinamide, or a pharmaceutically acceptable salt thereof.
F664. A heterobifunctional compound of N-(6-(dimethylamino)pyridazin-3-yl)-2-((8-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)octyl)amino)-6-(methylamino)nicotinamide, or a pharmaceutically acceptable salt thereof.
F665. A heterobifunctional compound of N-(6-(dimethylamino)pyridazin-3-yl)-2-((2-(2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethoxy)ethyl)amino)-6-(methylamino)nicotinamide, or a pharmaceutically acceptable salt thereof.
F666. A heterobifunctional compound of 2-((3-((4-((8-cyclopentyl-7-oxo-7,8-dihydropyrido[2,3-d]pyrimidin-2-yl)amino)cyclohexyl)amino)-3-oxopropyl)amino)-N-(6-(dimethylamino)pyridazin-3-yl)-6-(methylamino)nicotinamide, or a pharmaceutically acceptable salt thereof.
F667. A heterobifunctional compound of 2-((4-((4-((8-cyclopentyl-7-oxo-7,8-dihydropyrido[2,3-d]pyrimidin-2-yl)amino)cyclohexyl)amino)-4-oxobutyl)amino)-N-(6-(dimethylamino)pyridazin-3-yl)-6-(methylamino)nicotinamide, or a pharmaceutically acceptable salt thereof.
F668. A heterobifunctional compound of 2-((5-((4-((8-cyclopentyl-7-oxo-7,8-dihydropyrido[2,3-d]pyrimidin-2-yl)amino)cyclohexyl)amino)-5-oxopentyl)amino)-N-(6-(dimethylamino)pyridazin-3-yl)-6-(methylamino)nicotinamide, or a pharmaceutically acceptable salt thereof.
F669. A heterobifunctional compound of 2-((6-((4-((8-cyclopentyl-7-oxo-7,8-dihydropyrido[2,3-d]pyrimidin-2-yl)amino)cyclohexyl)amino)-6-oxohexyl)amino)-N-(6-(dimethylamino)pyridazin-3-yl)-6-(methylamino)nicotinamide, or a pharmaceutically acceptable salt thereof.
F670. A heterobifunctional compound of 2-((7-((4-((8-cyclopentyl-7-oxo-7,8-dihydropyrido[2,3-d]pyrimidin-2-yl)amino)cyclohexyl)amino)-7-oxoheptyl)amino)-N-(6-(dimethylamino)pyridazin-3-yl)-6-(methylamino)nicotinamide, or a pharmaceutically acceptable salt thereof.
F671. A heterobifunctional compound of 2-((8-((4-((8-cyclopentyl-7-oxo-7,8-dihydropyrido[2,3-d]pyrimidin-2-yl)amino)cyclohexyl)amino)-8-oxooctyl)amino)-N-(6-(dimethylamino)pyridazin-3-yl)-6-(methylamino)nicotinamide, or a pharmaceutically acceptable salt thereof.
F672. A heterobifunctional compound of 2-((2-(3-((4-((8-cyclopentyl-7-oxo-7,8-dihydropyrido[2,3-d]pyrimidin-2-yl)amino)cyclohexyl)amino)-3-oxopropoxy)ethyl)amino)-N-(6-(dimethylamino)pyridazin-3-yl)-6-(methylamino)nicotinamide, or a pharmaceutically acceptable salt thereof.
F673. A heterobifunctional compound of 2-((3-((2-((8-cyclopentyl-7-oxo-7,8-dihydropyrido[2,3-d]pyrimidin-2-yl)amino)spiro[3.5]nonan-7-yl)amino)-3-oxopropyl)amino)-N-(6-(dimethylamino)pyridazin-3-yl)-6-(methylamino)nicotinamide, or a pharmaceutically acceptable salt thereof.
F674. A heterobifunctional compound of 2-((4-((2-((8-cyclopentyl-7-oxo-7,8-dihydropyrido[2,3-d]pyrimidin-2-yl)amino)spiro[3.5]nonan-7-yl)amino)-4-oxobutyl)amino)-N-(6-(dimethylamino)pyridazin-3-yl)-6-(methylamino)nicotinamide, or a pharmaceutically acceptable salt thereof.
F675. A heterobifunctional compound of 2-((5-((2-((8-cyclopentyl-7-oxo-7,8-dihydropyrido[2,3-d]pyrimidin-2-yl)amino)spiro[3.5]nonan-7-yl)amino)-5-oxopentyl)amino)-N-(6-(dimethylamino)pyridazin-3-yl)-6-(methylamino)nicotinamide, or a pharmaceutically acceptable salt thereof.
F676. A heterobifunctional compound of 2-((6-((2-((8-cyclopentyl-7-oxo-7,8-dihydropyrido[2,3-d]pyrimidin-2-yl)amino)spiro[3.5]nonan-7-yl)amino)-6-oxohexyl)amino)-N-(6-(dimethylamino)pyridazin-3-yl)-6-(methylamino)nicotinamide, or a pharmaceutically acceptable salt thereof.
F677. A heterobifunctional compound of 2-((7-((2-((8-cyclopentyl-7-oxo-7,8-dihydropyrido[2,3-d]pyrimidin-2-yl)amino)spiro[3.5]nonan-7-yl)amino)-7-oxoheptyl)amino)-N-(6-(dimethylamino)pyridazin-3-yl)-6-(methylamino)nicotinamide, or a pharmaceutically acceptable salt thereof.
F678. A heterobifunctional compound of 2-((8-((2-((8-cyclopentyl-7-oxo-7,8-dihydropyrido[2,3-d]pyrimidin-2-yl)amino)spiro[3.5]nonan-7-yl)amino)-8-oxooctyl)amino)-N-(6-(dimethylamino)pyridazin-3-yl)-6-(methylamino)nicotinamide, or a pharmaceutically acceptable salt thereof.
F679. A heterobifunctional compound of 2-((2-(3-((2-((8-cyclopentyl-7-oxo-7,8-dihydropyrido[2,3-d]pyrimidin-2-yl)amino)spiro[3.5]nonan-7-yl)amino)-3-oxopropoxy)ethyl)amino)-N-(6-(dimethylamino)pyridazin-3-yl)-6-(methylamino)nicotinamide, or a pharmaceutically acceptable salt thereof.

In some embodiments, the monofunctional or heterobifunctional compounds described herein may compete for binding to DDB1 with one or more viral proteins or viral-derived peptides. In some embodiments, the monofunctional or heterobifunctional compound competitively binds to the same binding site on DBB1 as a viral protein or a viral-derived peptide. Such competitive binding can be measured with a competition binding assay and used to identify and characterize the residues comprising the DBB1 binding site and/or the binding affinity of the monofunctional or heterobifunctional compound. In some embodiments, the viral protein or viral-derived peptide comprises any of SEQ ID NOs:1-7, as shown in Table 5. In some embodiments, the viral-derived peptide is a peptide consisting essentially of any of SEQ ID NOs:1-7, or a labeled form thereof. Such peptides or labeled forms may be used in a competition binding assay for the monofunctional or heterobifunctional compounds described herein. In some embodiments, provided is a method of determining whether a test compound is capable of binding to DDB1, comprising the steps of: (1) contacting DDB1 with a radiolabeled viral protein or viral-derived peptide and the test compound, and (2) measuring reactivity bound to the DDB1.

TABLE 5
Peptide DDB1 binding moieties

| SEQ ID NO: | Peptide Sequence |
|---|---|
| 1 | ILPKVLHKRTLGLS |
| 2 | ILPKVWHKRELGLS |
| 3 | ILPKVLHKRTLGL |
| 4 | ILPKVLHKRTFGL |
| 5 | KVLHKRTLG |
| 6 | NFTSRLNRRASFP |
| 7 | SRLNRRASF |

In another aspect, provided herein is a method of degrading a protein of interest (POI), comprising contacting the POI with a heterobifunctional compound of any of the formulae described herein. In some embodiments, the contacting comprises contacting a cell comprising the POI with the heterobifunctional compound. In some embodiments, contacting the POI with the heterobifunctional compound comprises administering the heterobifunctional compound to a subject comprising the cell. In some embodiments, the contacting results in degradation of the target protein.

In another aspect, provided herein is a method of identifying a compound capable of degrading a POI, comprising the steps of: (1) providing a test system for monitoring degradation of the POI; and (2) determining whether a given test compound leads to degradation of the POI in said test system. In some embodiments, the test system comprises a cell. In some embodiments, the test system comprises a cancer cell. In some embodiments, the cell is in a subject.

In some embodiments of the foregoing aspects, the POI is selected from CCND, CDK4 and/or CDK6. In some such embodiments, the POI is CCND. In some such embodiments, the POI is CCND1. In some such embodiments, the POI is CCND3. In some such embodiments, the POI is CDK4. In some embodiments, degradation is determined by an immunoassay. In some embodiments, degradation is ubiquitin-mediated. In some embodiments, degradation is by a proteasome.

Also described herein are modified proteins and protein-ligand complexes. The modified proteins and protein-ligand complexes of some embodiments are useful for biotechnology applications such as selective degradation of a target protein, molecular glues, or anti-microbial drugs.

In some embodiments, the binding between the DDB1 binding moiety and the DDB1 protein is non-covalent. The binding may include a non-covalent bond. The binding may include more than one non-covalent bond. Some non-limiting examples of non-covalent bonds include a salt-bridge, a Coulombic interaction, a hydrogen bond, a stereoelectronic interaction, or a dispersion contact. The binding may include a combination of non-covalent bonds. In some embodiments, the binding between the DDB1 binding moiety and the DDB1 protein is covalent.

### Therapeutic Methods and Uses

Also provided herein are therapeutic methods and uses comprising administering a compound of any of Formulae (I)-(XII), or a pharmaceutically acceptable salt thereof, or a pharmaceutical composition comprising the compound or salt of any of Formulae (I)-(XII), alone or in combination with one or more other therapeutic agents or palliative agents.

In one aspect, provided herein is a method for the treatment of a disease or disorder associated with abnormal cell growth, such as cancer, in a subject in need thereof, comprising administering to the subject a therapeutically effective amount of a heterobifunctional compound of any of Formulae (I)-(XII), or a pharmaceutically acceptable salt thereof. In some embodiments, provided herein is a method for the treatment of cancer in a subject in need thereof, comprising administering to the subject a therapeutically effective amount of a heterobifunctional compound of any of Formulae (I)-(XII), or a pharmaceutically acceptable salt thereof. In some embodiments, the method further comprises administering an additional therapeutic agent (e.g., an anticancer therapeutic agent) to the subject.

In another aspect, provided is a method for the treatment of a disease or disorder associated with abnormal cell growth, such as cancer, in a subject in need thereof, comprising administering to the subject an amount of a heterobifunctional compound of any of Formulae (I)-(XII), or a pharmaceutically acceptable salt thereof, in combination with an amount of an additional therapeutic agent (e.g., an anticancer therapeutic agent), wherein the amounts are together effective in treating said abnormal cell growth, such as cancer.

Enumerated embodiments E62 to E76 relate to methods of treatment, uses, or compounds for use, comprising heterobifunctional compounds of any of Formulae (I)-(XII). Enumerated embodiments E77 to E85 relate to methods of degrading a POI, or methods of identifying compounds capable of degrading a POI, preferably comprising heterobifunctional compounds of any of Formulae (I)-(XII).

E62. A method for the treatment of a disease or disorder associated with abnormal cell growth, preferably cancer, in a subject in need thereof, comprising administering to the subject a therapeutically effective amount of a heterobifunctional compound of any one of embodiments E1 to E48, or a pharmaceutically acceptable salt thereof, or a pharmaceutical composition of embodiment E49.
E63. The method of embodiment E62, wherein the cancer is selected from breast cancer, ovarian cancer, endometrial cancer, uterine cancer, bladder cancer, prostate cancer, lung cancer, bone cancer, central nervous system (CNS) cancer, oral cancer, esophageal cancer, head and neck cancer, colorectal cancer, kidney cancer, liver cancer, pancreatic cancer, gastric cancer, thyroid cancer, melanoma, and a hematopoietic or lymphoid cancer.
E64. The method of embodiment E62 or E63, wherein the cancer is mediated by cyclin D (CCND), cyclin dependent kinase 4 (CDK4) and/or cyclin dependent kinase 6 (CDK6).
E65. The method of embodiment E64, wherein the cancer is mediated by cyclin D (CCND).
E66. The method of embodiment E65, wherein the cancer is mediated by cyclin D1 (CCND1).
E67. The method of embodiment E65, wherein the cancer is mediated by cyclin D3 (CCND3).
E68. The method of embodiment E62 or E63, wherein the cancer is characterized by amplification or overexpression of one or more of CCND, CDK4, or CDK6.
E69. A method to degrade one or more proteins selected from CCND, CDK4 or CDK6, comprising administering a heterobifunctional compound of any one of embodiments E1 to E48, or a pharmaceutically acceptable salt thereof, or a pharmaceutical composition of embodiment E49.
E70. The method of embodiment E69, wherein the method degrades the protein CCND.
E71. The method of embodiment E69, wherein the method degrades the proteins CCND and CDK4.
E72. The method of any one of embodiments E69 to E71, wherein the amount of the protein is decreased in a subject after administration of a compound according to any one of embodiments E1 to E48, or a pharmaceutically acceptable salt thereof, relative to a baseline measurement of the protein in the subject.
E73. A heterobifunctional compound of any one of embodiments E1 to E48, or a pharmaceutically acceptable salt thereof, or a pharmaceutical composition of embodiment E49, for use in the treatment of a disease or disorder associated with abnormal cell growth, preferably cancer.
E74. A heterobifunctional compound of any one of embodiments E1 to E48, or a pharmaceutically acceptable salt thereof, or a pharmaceutical composition of embodiment E49, for use in a method to degrade one or more proteins selected from CCND, CDK4 or CDK6.
E75. Use of a heterobifunctional compound of any one of embodiments E1 to E48, or a pharmaceutically acceptable salt thereof, for the manufacture of a medicament for the treatment of a disease or disorder associated with abnormal cell growth, preferably cancer.
E76. Use of a heterobifunctional compound of any one of embodiments E1 to E48, or a pharmaceutically acceptable salt thereof, for the manufacture of a medicament to degrade one or more proteins selected from CCND, CDK4 or CDK6.
E77. A method of degrading a protein selected from CCND, CDK4 and/or CDK6, comprising contacting the protein with a heterobifunctional compound of any one of embodiments E1 to E48, or a pharmaceutically acceptable salt thereof, or a pharmaceutical composition of embodiment E49, wherein the contacting comprises contacting a cell comprising the protein with the heterobifunctional compound and results in degradation of the target protein.
E78. A method of identifying a compound capable of degrading a protein selected from CCND, CDK4 and/or CDK6, comprising the steps of: (l) providing a test system for monitoring the degradation of the protein; and (2) determining whether a given test compound leads to degradation of the protein in said test system (wherein the test compound is preferably a heterobifunctional compound or salt of any one of embodiments E1 to E48, or a pharmaceutical composition of embodiment E49).
E79. The method of embodiment E78, wherein the test system comprises a cell.
E80. The method of embodiment E77 or E78, wherein the cell is a cancer cell.
E81. The method of embodiment E80, wherein the cancer cell is in a subject.
E82. The method of any one of embodiments E77 to E81, wherein the protein is CCND.
E83. The method of any one of embodiments E77 to E82, wherein the degradation is determined by an immunoassay.
E84. The method of any one of embodiments E77 to E83, wherein the degradation is ubiquitin-mediated.
E85. The method of any one of embodiments E77 to E84, wherein the degradation is by a proteasome.

In another aspect, provided is a heterobifunctional compound of any of Formulae (I)-(XII), or a pharmaceutically acceptable salt thereof, for use in the treatment of abnormal cell growth, such as cancer, in a subject.

In a further aspect, provided is the use of a heterobifunctional compound of any of Formulae (I)-(XII), or a pharmaceutically acceptable salt thereof, for the treatment of abnormal cell growth, such as cancer, in a subject.

In a further aspect, provided is the use of a heterobifunctional compound of any of Formulae (I)-(XII), or a pharmaceutically acceptable salt thereof, for use as a medicament, e.g., a medicament for the treatment of abnormal cell growth, such as cancer.

In a further aspect, provided is the use of a heterobifunctional compound of any of Formulae (I)-(XII), or a pharmaceutically acceptable salt thereof, for use in the manufacture of a medicament, including, e.g., a medicament for the treatment of abnormal cell growth, such as cancer.

In some embodiments of the compounds, compositions, methods and uses described herein, the heterobifunctional compound of any of Formulae (I)-(XII), or a pharmaceutically acceptable salt thereof, selectively degrades CCND over CDK4 and/or CDK6. In some embodiments, the heterobifunctional compound or salt of any of Formulae (I)-(XII) degrades both CCND and CDK4. In some embodiments, the heterobifunctional compound or salt of any of Formulae (I)-(XII) selectively degrades CCND and CDK4 relative to CDK6.

In frequent embodiments of each of the methods and uses herein, the abnormal cell growth is cancer. In some such embodiments, the cancer is mediated by CCND.

In some embodiments of any of the methods or uses herein, the disease or disorder associated with abnormal cell growth is cancer. In some embodiments of any of the methods or uses herein, the disease or disorder is cancer mediated by CCND, CDK4 and/or CDK6. In some embodiments of any of the methods or uses herein, the disease or disorder is cancer mediated by CCND. In some embodiments of any of the methods or uses herein, the disease or disorder is mediated by CCND1. In some embodiments of any of the methods or uses herein, the disease or disorder is mediated by CCND3. In some embodiments of any of the methods or uses herein, the disease or disorder is mediated by CCND2. In some embodiments of any of the methods or uses herein, the disease or disorder is mediated CCND1 and/or CCND3. In some embodiments of any of the methods or uses herein, the disease or disorder is cancer mediated by CDK4 and/or CDK6.

In some such embodiments, the cancer is characterized by amplification or overexpression of one or more of CCND, CDK4, or CDK6. In some such embodiments, the cancer is characterized by amplification or overexpression of CCND. In some such embodiments, the cancer is characterized by amplification or overexpression of CCND1. In some such embodiments, the cancer is characterized by amplification or overexpression of CCND3. In some such embodiments, the cancer is characterized by amplification or overexpression of CCND2. In some such embodiments, the cancer is characterized by amplification or overexpression of CCND1 and/or CCND3. In some such embodiments, the cancer is characterized by amplification or overexpression of CDK4 and/or CDK6.

In other embodiments, the cancer is characterized by amplification, overexpression, rearrangement, microdeletion, or mutation of CCND, including, e.g., CCND1 and/or CCND3.

In some embodiments of any of the methods and uses provided herein, the subject is identified as having a cancer characterized by amplification or overexpression of CCND. In some such embodiments, the subject is identified as having a cancer characterized by amplification or overexpression of CCND1. In some such embodiments, the subject is identified as having a cancer characterized by amplification or overexpression of CCND3. In some such embodiments, the subject is identified as having a cancer characterized by amplification or overexpression of CCND2. In some such embodiments, the subject is identified as having a cancer characterized by amplification or overexpression of CCND1 and/or CCND3.

In some embodiments of any of the methods and uses provided herein, the subject is identified as having a cancer characterized by amplification or overexpression of CDK4 and/or CDK6. In some such embodiments, the subject is identified as having a cancer characterized by amplification or overexpression of CDK4. In some such embodiments, the subject is identified as having a cancer characterized by amplification or overexpression of CDK6.

In another aspect, provided herein is a method inducing apoptosis of cancer cells in a subject in need thereof, comprising administering to the subject a heterobifunctional compound of any of Formulae (I)-(XII), or a pharmaceutically acceptable salt thereof, in an amount effective to activate apoptosis.

In some embodiments, provided herein is a heterobifunctional compound of any of Formulae (I)-(XII), or a pharmaceutically acceptable salt thereof, for use in a method of treating a disease or disorder associated with abnormal cell growth, such as cancer.

“Abnormal cell growth” refers to cell growth that is independent of normal regulatory mechanisms (e.g., loss of contact inhibition). Abnormal cell growth may be benign (not cancerous), or malignant (cancerous).

In some embodiments, provided herein is a method to (a) degrade, (b) inhibit, or (c) modulate one or more proteins selected from CCND (i.e., CCND1, CCND3 and/or CCND2), CDK4 or CDK6. In one embodiment, provided herein is a method to degrade one or more proteins selected from CCND (i.e., CCND1, CCND3 and/or CCND2), CDK4 or CDK6. In another embodiments, provided herein is a method to modulate one or more proteins selected from CCND (i.e., CCND1, CCND3 and/or CCND2), CDK4 or CDK6. In another embodiment, provided herein is a method to inhibit one or more proteins selected from CCND (i.e., CCND1, CCND3 and/or CCND2), CDK4 or CDK6. In a further embodiment, provided herein is a method to (a) degrade, (b) inhibit, or (c) modulate one or more proteins selected from CCND, CDK4 or CDK6, comprising administering to a subject in need thereof an effective amount of a heterobifunctional compound of any of Formulae (I)-(XII), or a pharmaceutically acceptable salt thereof. In some embodiments, the method degrades one or more proteins. In some embodiments, the method inhibits one or more proteins. In some embodiments, the method modulates one or more proteins, for example by reducing the activity of the protein, or by reducing the expression level of the protein or reducing the expression level of its corresponding gene.

In some embodiments, provided herein is a heterobifunctional compound of any of Formulae (I)-(XII), or a pharmaceutically acceptable salt thereof, thereof, for use in a method to degrade, inhibit, or modulate one or more proteins selected from CCND (i.e., CCND1, CCND3 and/or CCND2), CDK4 and CDK6. In preferred embodiments, provided is a method to selectively degrade, inhibit, or modulate a protein selected from CCND, CDK4 or CDK6. In preferred embodiments, the protein is CCND (including, e.g., CCND1, CCND3 and/or CCND2). In preferred embodiments, the method selectively degrades CCND (including, e.g., CCND1, CCND3 and/or CCND2).

In some embodiments of the foregoing methods and compounds for use, the protein is CCND. In some such embodiments, the protein is CCND1. In some such embodiments, the POI is CCND2. In other such embodiments, the protein is CCND3. In some embodiments of the foregoing methods and compounds for use, the protein is CDK4. In some embodiments of the foregoing methods and compounds for use, the proteins are CCND and/or CDK4. In some such embodiments, the proteins are CCND1 and/or CDK4. In some such embodiments, the proteins are CCND3 and/or CDK4. In some such embodiments, the proteins are CCND and CDK4. In some such embodiments, the proteins are CCND1 and CDK4. In some such embodiments, the proteins are CCND3 and CDK4. In some embodiments, the method or compound for use selectively degrades CCND (relative to CDK4 and/or CDK6). In some embodiments, the method or compound for use degrades both CCND and CDK4. In some such embodiments, CDK4 is degraded more slowly than CCND. In some such embodiments, CDK4 is degraded to a lesser extent than CCND. In some embodiments, the compound of CCND is degraded while CDK4 is not degraded.

In some embodiments wherein the protein is degraded, inhibited, or modulated, respectively, the amount of the protein(s) is decreased in the subject after administration of a compound described herein relative to a baseline measurement. The amount of the protein(s) may be measured in a test sample (e.g., a tissue sample or fluid sample) obtained from a subject following administration, and the amount may be determined relative to a baseline measurement for the protein(s) in a first sample obtained from the subject prior to administration of said compound. Some embodiments include measuring a decrease in the amount of one or more protein(s) following the administration.

In some embodiments, the method further comprises comparing the amount of the protein(s) in a first sample obtained from a subject prior to administration of a compound described herein, and the amount of the protein(s) in a test sample (i.e., a second or later sample) obtained from the subject (at a relevant timepoint) after administration of said compound. Where the protein is degraded by the compound of interest, the amount of the protein(s) in the test sample is expected to be decreased relative to a baseline measurement from the first sample. The extent of the decrease may be greater than: 10%, 20%, 30%, 40%, 50%, 60%, 70%, 80%, or 90% at relevant timepoints. Each such sample may be a tissue sample or a fluid sample.

The measurement of the amount of a protein in a first (baseline) sample and/or a test (treatment) sample may be obtained directly in the subject. The measurement may include a concentration. The measurement may be normalized, for example to a sample weight, to a sample volume, to a total sample protein measurement, or to a housekeeping protein measurement. Measurement of the protein in the test sample obtained at defined timepoint(s) after administration of the compound described herein, during administration of the compound described herein to a subject.

Measurements of test and baseline levels of POIs may include any method known in the art. For example, such measurements may be obtained using an assay such as an immunoassay, a colorimetric assay, a lateral flow assay, a fluorescence assay, a proteomics assay, or a cell-based assay. The immunoassay may include an immunoblot such as a Western blot or a dot blot, an enzyme-linked immunosorbent assay, or immunostaining. The proteomics assay may include mass spectrometry. Such measurements may be obtained using flow cytometry. Such measurements may also be obtained using chromatography, for example high performance liquid chromatography.

In some embodiments of any of the methods and uses herein, the disease or disorder is cancer. Cancer includes solid tumors or cancers of the blood, bone marrow, or lymphatic system (sometimes referred to as “liquid tumors”). “Solid tumors” refer to abnormal masses of cells that grow in an organ system and generally do not contain cysts or liquid areas. Solid tumors may be noncancerous (benign) or cancerous (malignant) and may include a single mass or multiple masses. Cancerous solid tumors are typically named for the type of cells that form them, and include, but are not limited to, sarcomas and carcinomas. “Liquid tumors” may circulate throughout the body via the bloodstream. Cancers of the blood include, but are not limited to, leukemia, lymphoma, or myeloma. Cancer may include a primary cancer that originates at a specific site in the body, a metastatic cancer that has spread from the place in which it started to other parts of the body, a recurrence from the original primary cancer after remission, or a second primary cancer in a person with a prior history of cancer of a different type.

In some such embodiments, the cancer is selected from breast cancer, ovarian cancer, endometrial cancer, uterine cancer, bladder cancer, prostate cancer, lung cancer (e.g., NSCLC, SCLC, squamous cell carcinoma or adenocarcinoma), bone cancer, central nervous system (CNS) cancer, oral cancer, esophageal cancer, head and neck cancer (e.g., HNSSC), colorectal cancer, kidney cancer (e.g., RCC), liver cancer (e.g., HCC), pancreatic cancer, gastric cancer, thyroid cancer, melanoma, and a hematopoietic or lymphoid cancer (e.g., mantle cell lymphoma (MCL) or multiple myeloma). In some such embodiments, the cancer is characterized by amplification or overexpression of CCND, such as CCND1 and/or CCND3.

In some embodiments of each of the methods and uses described herein, a compound or composition as described herein may be administered as a single agent or in combination with one or more additional therapeutic agents, e.g., an additional therapeutic agent that is a standard of care agent for the disease or disorder being treated, such as an anti-cancer therapeutic agent appropriate for a particular cancer.

In some embodiments, the compound or composition is administered in combination with an endocrine therapy agent (e.g., letrozole, fulvestrant, elacestrant, tamoxifen, exemestane, or anastrozole). In some embodiments, the compound or composition is administered in combination with a chemotherapeutic agent (e.g., docetaxel, paclitaxel, cisplatin, carboplatin, capecitabine, gemcitabine, vinorelbine, and the like).

In some embodiments, the cancer is characterized by primary or acquired resistance to treatment with one or more standard of care agents for a particular cancer. In some embodiments, the cancer is advanced or metastatic cancer.

In some embodiments of each of the methods and uses herein, the cancer is breast cancer. In some embodiments, the breast cancer is hormone receptor positive (HR+), human epidermal growth factor 2 (HER2)-negative breast cancer. In some embodiments, the breast cancer is triple negative breast cancer (TNBC). In some embodiments, the breast cancer is advanced or metastatic breast cancer. In some embodiments, the breast cancer is HR+, HER2-negative advanced or metastatic breast cancer. In some embodiments, the cancer has progressed on prior treatment with CDK4/6 inhibitors and/or endocrine therapy. In some embodiments, the cancer demonstrates primary or acquired resistance to treatment with CDK4/6 inhibitors and/or endocrine therapy.

In some embodiments of any of the methods and uses herein, the cancer is ovarian cancer, endometrial cancer, or uterine cancer. In some embodiments, the cancer is ovarian cancer. In some embodiments, the cancer is endometrial cancer. In some embodiments, the cancer is uterine cancer. In some embodiments, the ovarian cancer, endometrial cancer, or uterine cancer, respectively, is characterized by amplification or overexpression of CCND, such as CCND1 and/or CCND3.

In some embodiments of any of the methods and uses herein, the cancer is bladder cancer.

In some embodiments of any of the methods and uses herein, the cancer is prostate cancer. In some embodiments, the prostate cancer is characterized by amplification or overexpression of CCND, such as CCND1 and/or CCND3.

In some embodiments of any of the methods and uses herein, the cancer is lung cancer. In some embodiments, the lung cancer is characterized by amplification or overexpression of CCND, such as CCND1 and/or CCND3. In some embodiments, the lung cancer is NSCLC, SCLC, squamous cell carcinoma or adenocarcinoma. In some such embodiments, the lung cancer is NSCLC. In some such embodiments, the NSCLC is characterized by amplification or overexpression of CCND, such as CCND1 and/or CCND3. In some embodiments, the NSCLC is KRAS mutant NSCLC. In some embodiments, the lung cancer is lung adenocarcinoma (LUAD).

In some embodiments of any of the methods and uses herein, the cancer is bone cancer. In some embodiments, the bone cancer is primary bone cancer. In some embodiments, the bone cancer is bone metastasis. In some embodiments, the bone cancer is sarcoma. In some such embodiments, the sarcoma is Ewing sarcoma or rhabdomyosarcoma. In some embodiments, the methods and uses herein may reduce or ameliorate bone cancer pain (BCP). In some embodiments, the bone cancer is characterized by amplification or overexpression of CCND, such as CCND1 and/or CCND3.

In some embodiments of any of the methods and uses herein, the cancer is a nervous system cancer. In some embodiments, the cancer is central nervous system (CNS) cancer. In some embodiments the CNS cancer is glioma. In some such embodiments, the glioma is astrocytoma, oligodendroglioma, or glioblastoma. In some embodiments, the cancer is a sympathetic nervous system cancer. In some such embodiments, the nervous system cancer is neuroblastoma. In some embodiments, the nervous system cancer is characterized by amplification or overexpression of CCND, such as CCND1 and/or CCND3.

In some embodiments of any of the methods and uses herein, the cancer is oral cancer, esophageal cancer, or head and neck cancer. In some embodiments, the cancer is oral cancer. In some embodiments, the cancer is esophageal cancer. In some such embodiments, the esophageal cancer is ESCC, including, e.g., CDK4 amplified ESCC. In some embodiments, the cancer is head and neck cancer. In some such embodiments, the head and neck cancer is HNSSC. In some such embodiments, the HNSSC is HPV-negative HNSCC. In some such embodiments, the HNSSC is HPV-positive HNSCC. In some embodiments, the oral cancer, esophageal cancer, or head and neck cancer, respectively, is characterized by amplification or overexpression of CCND, such as CCND1 and/or CCND3. In some such embodiments, the cancer is HNSSC characterized by amplification or overexpression of CCND, such as CCND1 and/or CCND3.

In some embodiments of any of the methods and uses herein, the cancer is colorectal cancer, kidney cancer, liver cancer, pancreatic cancer, gastric cancer, or thyroid cancer. In some embodiments, the cancer is colorectal cancer. In some embodiments, the cancer is kidney cancer, e.g., RCC. In some embodiments, the cancer is liver cancer, e.g., HCC. In some embodiments, the cancer is pancreatic cancer. In some embodiments, the cancer is gastric cancer. In some embodiments, the cancer is thyroid cancer. In some embodiments the colorectal cancer, kidney cancer, liver cancer, pancreatic cancer, gastric cancer, or thyroid cancer, respectively, is characterized by amplification or overexpression of CCND, such as CCND1 and/or CCND3.

In some embodiments of any of the methods and uses herein, the cancer is melanoma. In some such embodiments, the melanoma is characterized by amplification or overexpression of CCND, such as CCND1 and/or CCND3.

In some embodiments of any of the methods and uses herein, the cancer is a hematopoietic or lymphoid cancer. In some embodiments, the hematopoietic or lymphoid cancer is lymphoma, myeloma, or leukemia. In some such embodiments, the hematopoietic or lymphoid cancer is characterized by amplification or overexpression of CCND, such as CCND1 and/or CCND3. In some such embodiments, the cancer is mantle cell lymphoma (MCL). In some such embodiments, the MCL is characterized by amplification or overexpression of CCND, such as CCND1 and/or CCND3. In some such embodiments, the cancer is multiple myeloma. In some such embodiments, the multiple myeloma is characterized by amplification or overexpression of CCND, such as CCND1 and/or CCND3.

In some embodiments, the methods and uses herein relate to treatment of (1) tumors or tumor cells having increased expression of CCND, CDK4 and/or CDK6; (2) tumors or tumor cells that proliferate by aberrant CCND, CDK4 and/or CDK6 activation; (3) tumors or tumor cells characterized by amplification or overexpression of CCND1, CCND2 and/or CCND3; (4) tumors or tumor cells characterized by amplification or overexpression of CDK4 and/or CDK6; or (5) tumors or tumor cells that are resistant to endocrine therapy and/or CDK4/6 inhibition.

In some embodiments, of any of the methods and uses herein, the heterobifunctional compound of any of the formulae herein is administered as first line therapy. In other embodiments, the heterobifunctional compound of any of the formulae herein is administered as second (or later) line therapy.

In some embodiments, the heterobifunctional compound is administered as second (or later) line therapy following treatment with, or progression on, one or more standard of care agent(s) for the disease or disorder, such as an anti-cancer therapeutic agent appropriate for a particular cancer. In some embodiments, the heterobifunctional compound is administered as second (or later) line therapy following treatment with, or progression on, one or more chemotherapy agents. In some embodiments, the heterobifunctional compound is administered as second (or later) line therapy following treatment with, or progression on, one or more targeted anti-cancer therapeutic agents.

In some embodiments, the cancer is breast cancer and the heterobifunctional compound is administered as second (or later) line therapy following treatment with, or progression on, an endocrine therapeutic agent and/or a CDK4/6 inhibitor. In some such embodiments, the heterobifunctional compound is administered as second (or later) line therapy following treatment with, or progression on, an endocrine therapeutic agent (e.g., an aromatase inhibitor, a SERM or a SERD). In some such embodiments, the heterobifunctional compound is administered as second (or later) line therapy following treatment with, or progression on, a CDK4/6 inhibitor.

In another aspect, provided herein is a method of degrading a target protein or POI in a sample by contacting the sample with a compound or salt of any of the formulae herein, or pharmaceutical composition comprising such compound or salt, thereby degrading the target protein or POI. In some embodiments, the sample is a biological sample. In some embodiments, the biological sample comprises a tissue, a cell, or a biological fluid. In some such embodiments, the biological sample is a cell. In some embodiments, the cell is a eukaryotic cell. In some embodiments, the cell is a mammalian cell. In some embodiments, the cell is a human cell. In some embodiments, the cell is a cancer cell. In some embodiments, the cell is a cancer cell collected from a subject, e.g., via biopsy. In some embodiments, the contacting is in vitro. In some embodiments, the contacting is in vivo. In some embodiments, upon being contacted with the compound or composition, the target protein or POI is ubiquitinated to form a ubiquitinated protein. In some embodiments, upon administration or contact, the ubiquitinated protein is degraded. In some embodiments, the degradation of the target protein or POI is selective for the target protein or POI. In some embodiments, the target protein or POI comprises proteasomal degradation. In some embodiments, the target protein or POI is degraded by the proteasome.

In another aspect, the compounds or compositions described herein may be used as antimicrobial drugs. In one aspect, provided herein is a method of treating a microbial infection comprising administering an effective amount of a compound of any of formulae disclosed herein to a subject in need thereof. The method may reduce the severity of the microbial infection in the subject, relative to a baseline measurement. The compound may bind a target protein involved in the microbial infection, resulting in inhibition or degradation of the target protein. The microbial infection may include a virus infection. The microbial infection may include a bacterial infection. The compound may be a heterobifunctional compound of any of the formulae herein where the target protein or POI is a microbial protein. The microbial protein may include a viral protein. The microbial protein may include a bacterial protein. The target protein or POI may be a non-microbial protein that exacerbates the microbial infection. The target protein or POI may be a non-microbial protein that prevents or decreases inhibition of the microbial infection. In some embodiments, the compound enters a cell of the subject, binds to a microbial protein in the cell via its

(PBM), binds to DDB1 via its DDB1 binding moiety, and induces ubiquitin-mediated degradation of the microbial protein. Such an action may be useful against microbes such as bacteria or viruses that infect or reside within the cell.

In some embodiments, a compound or composition described herein may be useful for modulating DDB1 protein levels. For example, the compounds or compositions may be used to increase DDB1 protein levels or decrease DDB1 protein levels. In some embodiments, the compound increases DDB1 protein levels. For example, the compound may bind to DDB1 and prevent its degradation. In some embodiments, the compound decreases DDB1 protein levels. For example, the compound may bind to DDB1 and increase its degradation.

In some embodiments, a compound or composition described herein may increase DDB1 activity or decrease DDB1 activity in a subject, relative to a baseline measurement. In some embodiments, the compound increases DDB1 activity. In some embodiments, the compound or composition decreases or inhibits DDB1 activity. In some embodiments, the compound allosterically activates or allosterically inhibits DDB1. In some embodiments, the compound activates or inhibits DDB1 by binding to a protein binding site on DDB1. In some embodiments, a compound or composition described herein may be useful for modulating DDB1 function. For example, the compound or composition may activate or inhibit DDB1.

Some embodiments of any of the methods and uses herein include a step of measuring the amount of a cyclin or a CDK in the cell. In some embodiments, the interaction between the cyclin and the CDK comprises binding or dimerization. In some such embodiments, the cyclin is CCND. In some such embodiments, the CDK is CDK4 and/or CDK6. In some embodiments, CCND is CCND1, CCND2, or CCND3. In some embodiments, the CCND is CCND1. In some embodiments, the CCND is CCND2. In some embodiments, the CCND is CCND3. In some embodiments, the CDK comprises CDK4. In some embodiments, the CDK comprises CDK6. In some embodiments, the CDK comprises CDK4 and CDK6 (CDK4/6).

“Abnormal cell growth” refers to cell growth that is independent of normal regulatory mechanisms (e.g., loss of contact inhibition). Abnormal cell growth may be benign (not cancerous), or malignant (cancerous).

The term “additional anticancer therapeutic agents” refers to one or more therapeutic agent, other than a compound described herein, that can be used in the treatment of cancer. In some embodiments, such additional anticancer therapeutic agents may include one or more: mitotic inhibitors, alkylating agents, antimetabolites, antitumor antibiotics, anti-angiogenesis agents, topoisomerase I and II inhibitors, plant alkaloids, hormone or endocrine therapy agents (such as aromatase inhibitors, SERMs or SERDs), growth factor inhibitors, radiation therapy, signal transduction inhibitors (such as tyrosine or serine/threonine kinase inhibitors), cell cycle inhibitors (such as CDK inhibitors), biological response modifiers (such as immunotherapy agents or immune checkpoint inhibitors), enzyme inhibitors, antisense oligonucleotides or derivatives, cytotoxic agents, and the like. The terms “ameliorate” or “ameliorating” mean a lessening or improvement of one or more symptoms of a disease or disorder upon treatment with a compound of composition as described herein, as compared to not administering the compound of composition.

The terms “cancer” or “cancerous” refer to any malignant and/or invasive growth or tumor caused by abnormal cell growth, typically including the rapid, uncontrolled or invasive growth of aberrant cells.

The terms “effective amount” or “therapeutically effective amount” refer to the amount of a compound or pharmaceutical composition as described herein, alone or in combination with one or more other agents, that is sufficient to achieve a desired or beneficial biological result, e.g., the amount sufficient to affect the biochemical, histological, or behavioral symptoms of a disease or disorder, its complications, or pathological phenotypes.

In some embodiments, the “therapeutically effective amount” is the amount of a compound or composition that, when administered to a subject, achieves a therapeutic effect relevant for the disease or disorder. In some embodiments, when the disease or disorder is cancer, the therapeutically effective amount is the amount that: (1) treats or prevents the disease or disorder; (2) ameliorates or eliminates one or more symptoms of the disease or disorder; or (3) prevents or delays the onset, progression, or recurrence of one or more symptoms of the disease or disorder.

In other embodiments, the effective amount is the amount of a compound, salt, or composition that, when administered to a cell, a tissue, a system, or a non-cellular biological material or medium, is effective to at least partially inhibit or degrade a POI, inhibit or reduce the activity of a POI, or reduce the expression level of a POI or its corresponding gene.

The terms “prevent”, “preventing” or “prevention” refer to the prophylactic treatment of a disease or disorder, or to delaying the onset, progression or recurrence of the disease or disorder.

As used herein, a “subject” refers to a human or non-human animal subject. Examples of subjects include humans and other mammals, such as dogs, cats, cattle, mice, rats, monkeys, or other non-human primates. In some preferred embodiments, the subject is a human. Subjects may include, e.g., human or veterinary patients, or human or veterinary subjects participating in clinical trials.

The terms “treat” or “treating” as used herein means to administer a compound, salt, or composition, as described herein, to a subject having a disease or disorder, such as cancer, to achieve at least one positive therapeutic effect. Such therapeutic effects may include reversing, relieving, alleviating, or slowing the progression of, or any damage associated with any symptoms of the disease or disorder. The term “treatment”, as used herein, unless otherwise indicated, refers to the act of treating as “treating” as defined above.

For the treatment of cancer, a therapeutic (or “anti-cancer”) effect may include a decrease in the number of cancer cells; a decrease in tumor size or volume; a decrease in the rate of cancer cell infiltration into peripheral organs; a decrease in the rate or number of metastases; a decrease in rate of tumor growth. In some embodiments of any of the methods or uses herein wherein the disease or disorder is cancer, the method or use results in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

Additional beneficial effects in treating cancer may include: decreasing or ameliorating at least one symptom of the cancer; increasing the quality of life of the subject having cancer; decreasing the dose, or enhancing the effectiveness, of another agent used to treat the cancer; inducing remission of the cancer; delaying the progression or recurrence of the cancer; curing the cancer; overcoming one or more resistance mechanisms of the cancer; and/or prolonging survival of the subject having cancer.

Positive therapeutic effects in cancer can be measured in several ways, including by measuring a reduction in tumor growth or tumor volume. In some embodiments, treatment results in reduction in tumor growth and/or tumor volume by at least about 20%, at least about 30%, at least about 40%, at least about 50%, at least about 60%, at least about 70%, or at least about 80%, relative to untreated subjects. The effect may also be determined in animal model systems predictive of efficacy in human tumors. Alternatively, the effect may be evaluated by measuring responses using in vitro, ex vivo, or in vivo assays known to the skilled practitioner. For example, in some embodiments the tumor growth inhibition T/C ratio may be used to quantify treatment effects in tumor xenograft experiments. (J. Wu, Statistical Inference for Tumor Growth Inhibition T/C Ratio, J. Biopharm. Stat. 2010, 20:954-964.)

In clinical studies, efficacy can be determined by analyzing data obtained from well-designed clinical trials using appropriate biostatistical methods known in the art (e.g., T-test, ANOVA, Chi-squared test, Fisher's exact test, Kaplan-Meier curves, log-rank test, Cox proportional hazards model, linear or logistic regression, Mann-Whitney U test, sign test, Wilcoxon signed-rank test, or Kruskal-Wallis test). Biostatistical methods may be applied to unadjusted or adjusted findings, and analyze continuous, binary, or time-to-event outcomes. The effect of treatment may be defined by reference to partial response (PR), complete response (CR), overall response (OR), progression free survival (PFS), disease free survival (DFS) and overall survival (OS). Evaluation of PR, CR, PFS, DFS, OR OS may be assessed using Response Evaluation Criteria in Solid Tumors (RECIST) 1.1 response criteria.

### Pharmaceutical Compositions, Medicaments, and Kits

A “pharmaceutical composition” refers to an admixture of one or more compounds, or a salt, solvate, or prodrug thereof, and at least one pharmaceutically acceptable excipient. Such compositions are sometimes referred to in the alternative as medicaments.

In one aspect, provided herein is a pharmaceutical composition comprising a compound of any of Formulae (I)-(XII), or a pharmaceutically acceptable salt thereof, and at least one pharmaceutically acceptable excipient. In some embodiments, the pharmaceutical composition comprises two or more pharmaceutically acceptable carriers and/or excipients.

In some embodiments, the pharmaceutical composition comprises a compound of any of Formulae (I)-(XII), or a pharmaceutically acceptable salt thereof, and at least one additional therapeutic agent. In some embodiments, the additional therapeutic agent is an additional anti-cancer agent. In some embodiments, the compound or salt of any of Formulae (I)-(XII) is co-formulated with an additional therapeutic agent, such as an additional anti-cancer agent (i.e., a fixed dose combination). In some embodiments, the compound or salt of any of Formulae (I)-(XII) is packaged with an additional therapeutic agent, such as an additional anti-cancer agent.

In some embodiments, the pharmaceutical composition further comprises at least one additional therapeutic agent. In some embodiments, the pharmaceutical composition further comprises at least one additional therapeutic agent (e.g., a fixed dose combination). In some embodiments, the pharmaceutical composition further comprises at least one additional anti-cancer agent.

As used herein, the term “combination therapy” refers to the administration of a compound of the invention together with an at least one additional therapeutic agent (e.g., an additional anti-cancer agent). The two agents may be administered sequentially or simultaneously, in any order. In some embodiments, the compound of the invention is administered prior to the additional therapeutic agent. In other embodiments, the compound of the invention is administered after the additional therapeutic agent. In other embodiments, the compound of the invention and the additional therapeutic agent are administered simultaneously. When administering a combination therapy, the two or more agents can be administered with independent frequencies, dosing regimens and/or routes of administration.

In some embodiments of any of the methods and uses herein, the compound of any of Formulae (I)-(XII), or a pharmaceutically acceptable salt thereof, can be administered in the form of a pharmaceutical composition.

In some embodiments provided herein is the use of a compound of any of Formulae (I)-(XII), or a pharmaceutically acceptable salt thereof, for the manufacture of a medicament. In some such embodiments, the medicament if for use in the treatment of a disease or disorder associated with abnormal cell growth, such as cancer.

In some embodiments provided herein is a compound of any of Formulae (I)-(XII), or a pharmaceutically acceptable salt thereof, for use in the manufacture of a medicament.

In some embodiments provided herein is a compound of any of Formulae (I)-(XII), or a pharmaceutically acceptable salt thereof, for use in the treatment of a disease or disorder associated with abnormal cell growth, such as cancer.

The term “pharmaceutically acceptable” means the compound, salt, or pharmaceutical composition is suitable for administration to a subject.

The term “pharmaceutically acceptable excipients” refers to substances other than the active pharmaceutical ingredient (“API”) included in a pharmaceutical formulation or medicament, which are compatible with the other ingredients of the composition, are not deleterious to the subject, and do not abrogate the biological activity and properties of the API. Excipients, such as fillers or diluents, may bulk-up the formulation, allowing convenient and accurate dispensation of a drug substance when producing a dosage form. Excipients may also facilitate drug absorption, solubility, or other pharmacokinetic properties, or enhance stability of the drug product. In the manufacturing process, excipients may improve handling of the API (e.g., by facilitating powder flowability or non-stick properties). The choice of excipient(s) also depends on factors such as the intended route of administration, the effect of the excipient on solubility and stability, the nature of the dosage form, and standard pharmaceutical practice. See, e.g., Remington: The Science and Practice of Pharmacy (Gennaro, 21^{st} Ed Mack Pub. Co., Easton, PA (2005)).

The pharmaceutical composition maybe in a dosage form suitable for oral administration (e.g., a tablet or capsule), for parenteral injection (e.g., a sterile solution, suspension, or emulsion), for topical administration (e.g., an ointment or cream), or for rectal administration (e.g., a suppository).

Formulations suitable for oral administration include solid dosage forms, such as tablets, capsules (containing particulates, liquids, or powders), lozenges (including liquid-filled), chews, etc. Liquid formulations for oral administration include suspensions, solutions, syrups, or elixirs. Such formulations may also be used as fillers in soft or hard capsules, typically including a carrier (e.g., water, ethanol, polyethylene glycol, propylene glycol, methylcellulose, or a suitable oil), an emulsifying agent, and/or a suspending agent. Liquid formulations may also be prepared by the reconstitution of a solid formulation by addition of appropriate solvent or carrier.

The concentration of various excipients in a pharmaceutical formulation may be expressed as a ratio or percentage of the excipient to the drug product, by weight or volume. For solid dosage forms, concentrations are frequently described as the weight percent (wt %) of the total weight, or as the ratio of weight in weight (denoted as w/w). The concentration of a solid ingredient in a liquid vehicle may be expressed as the ratio of weight in volume (denoted as w/v). Alternatively, the concentration of a liquid ingredient in a solid vehicle may be expressed as the ratio of volume in weight (denoted as v/w). If both ingredients are liquids, the ratio may be expressed as the ratio of volume in volume (denoted as v/v).

Frequently used excipients in solid dosage forms include diluents, binders, glidants, disintegrants, lubricants, or anti-oxidants. Such dosage forms may also include release modifiers (e.g., pH modifiers), colorants, sweeteners, flavorants, preservatives, suspending agents, emulsifiers, or coating agents (e.g., film coating agents).

For tablet dosage forms, depending on dose, the API may make up from 1 wt % to 80 wt % of the dosage form, more typically from 5 wt % to 60 wt % of the dosage form. In addition to the API, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, more typically from 5 wt % to 20 wt % of the dosage form.

Binders or binding agents may be used to impart cohesive qualities to a tablet formulation. Suitable binders include microcrystalline cellulose, gelatin, sugars, polyethylene glycol, natural and synthetic gums, polyvinylpyrrolidone (e.g., povidone k-30), pregelatinized starch, carboxymethyl cellulose, sodium cellulose, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch, pregelatinized starch, sugar, calcium carbonate, calcium phosphate, dibasic calcium phosphate dihydrate, or tribasic calcium sulfate cellulose.

Tablets may also optionally include surface active agents (e.g., wetting, dispersion, or emulsion agents), such as sodium lauryl sulfate and polysorbate 80, and glidants such as silicon dioxide, magnesium silicate, calcium silicate and tale. When present, surface active agents are typically present in amounts from 0.2 wt % to 5 wt % of the tablet, and glidants are typically present in amounts from 0.2 wt % to 1 wt % of the tablet.

Tablets may also contain lubricants, such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, more typically from 0.5 wt % to 3 wt % of the tablet.

Other conventional ingredients include anti-oxidants (e.g., butyl hydroxyl toluene, butyl hydroxyl anisole, ascorbic acid, etc.), colorants, flavoring agents, preservatives (e.g., benzyl alcohol, sodium benzoate, paraben esters, chlorocresol, etc.), taste-masking agents, and coating agents (e.g., carboxymethyl cellulose, cellulose acetate, cellulose acetate phthalate (CAP), ethyl cellulose, hydroxypropyl methylcellulose (HPMC), hydroxypropyl methyl cellulose phthalate, methacrylic acid copolymer, methyl cellulose, PEG, polyvinyl acetate, Shellac, titanium dioxide, wax, Carnauba wax, microcrystalline zein, etc.

Exemplary tablets contain up to about 80 wt % API, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated (e.g., film coated), uncoated, or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid compositions may also be employed in soft and hard filled gelatin capsules. Non-limiting examples of excipients include lactose or milk sugar and high molecular weight polyethylene glycols. Capsules (made, for example, from gelatin or HPMC), blisters and cartridges may be formulated to contain a powder mix of the compound of the invention, a suitable powder base such as lactose (e.g., anhydrous lactose or lactose monohydrate) or starch and a performance modifier such as I-leucine, mannitol, or magnesium stearate. Other suitable excipients include dextran, glucose, maltose, sorbitol, xylitol, fructose, sucrose, and trehalose.

For oral aqueous suspensions or solutions, the API may be combined with various sweetening or flavoring agents, colorants, emulsifying agents or suspending agents, together with diluents such as water, ethanol, propylene glycol, glycerin, or combinations thereof. Frequently used excipients in oral liquid dosage formulations include solvents (e.g., water, alcohol, acetic acid, etc.), co-solvents (e.g., ethanol, sorbitol, glycerin, propylene glycol, etc.), buffers (e.g., phosphate, acetate or citric acid phosphate buffers), antimicrobial preservatives (e.g., benzyl alcohol, sodium benzoate, paraben esters, etc.), antioxidants (e.g., ascorbic acid, sodium bisulphate, thiourea, BHT, tocopherols, etc.), wetting agents (e.g., sodium lauryl sulphate, Tween 80, Spans, lecithins, etc.), emulsifying agents (e.g., sodium lauryl sulphate, PEG esters, sorbitan esters, etc.), or sweeteners (e.g., sucrose, sorbitol, saccharin, aspartame, sucralose, etc.).

Exemplary parenteral administration forms include solutions or suspensions of active compounds in sterile aqueous solutions, for example, aqueous propylene glycol or dextrose solutions. Such dosage forms may be buffered, if desired. Parenteral formulations are typically aqueous solutions which may contain excipients such as salts, carbohydrates, and buffering agents (preferably to a pH of from 3 to 9).

For some applications, parenteral formulations may be formulated as a sterile non-aqueous solution or as a dried form to be reconstituted and used in conjunction with a suitable vehicle such as sterile, pyrogen-free water.

The preparation of parenteral formulations under sterile conditions, for example, by lyophilization, may readily be accomplished using standard pharmaceutical techniques known to those skilled in the art. The solubility of compounds or salts as used in the preparation of parenteral solutions may be increased by appropriate formulation techniques, such as the incorporation of solubility-enhancing agents. Also provided herein are kits comprising two or more pharmaceutical compositions suitable for coadministration of the compositions. Typically, the kit includes two or more separate pharmaceutical compositions, at least one of which contains a compound or salt of any of the formulae disclosed herein, and means for separately retaining said compositions, such as a container, divided bottle, or divided foil packet. An example of such a kit is the familiar blister pack used for the packaging of tablets, capsules, and the like. Kits are particularly suitable for administering different dosage forms, for example, oral and parenteral, for administering the separate compositions at different dosage intervals, or for titrating the separate compositions against one another. To assist compliance, kits typically includes written directions for use and may be provided with a memory aid.

### Dosage Forms and Dosing Regimens

The compounds described herein may be administered by any method which delivers the compound systemically and/or locally to the intended site of action. Exemplary methods of administration include oral, parenteral (e.g., intravenous, intramuscular, subcutaneous, intraperitoneal, intravascular, or intramedullary injection or infusion), intraduodenal, rectal, buccal, intranasal, topical, transdermal, etc.

The dose and dosing regimen for a compound or composition as described herein, including the frequency of administration, the total dose, the dose administered per administration, the time interval between administrations, and the duration or treatment, may vary according to factors such as the disease state, age, and weight of the patient, and the ability of the therapy to elicit a response in the subject.

An effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, typically about 0.7 to about 2500 mg/day. In some instances, dosage levels below the lower limit of the range above may be sufficient, while in other cases still larger doses may be used without causing harmful side effects, with larger doses typically divided into several smaller doses for administration throughout the day.

A “dosing regimen” or “dosing schedule” refers to the dose and timing of administration of the compounds or compositions described herein comprising one or more treatment cycles, wherein each cycle can include administration of one or more agents described herein at different times or in different amounts, alone or in combination with another therapeutic agent. Dosing regimens may be adjusted to provide the desired prophylactic or therapeutic response. An effective dose can be administered in one or more administrations. The total dose may be administered as a single dose or divided into several doses and administered over time. In some embodiments, a compound or composition is administered once per day (QD), twice per day (BID), or three times per day (TID). In some embodiments, the compound or composition is administered on a continuous dosing regimen comprising daily administration, during one or more treatment cycles. In some embodiments, the compound or composition is administered on an intermittent dosing regimen comprising administration on one or more consecutive days followed by one or more consecutive days of rest on which the compound is not administered, during one or more treatment cycles. In certain embodiments, the compound or composition is administered orally. The dose may be reduced or increased proportionally as appropriate for the circumstances.

For convenience, the compounds described herein can be formulated and administered in unit dosage forms. “Unit dosage forms” refer to physically discrete units suited as unitary dosages for the subjects to be treated. Each unit dosage form contains a predetermined quantity of the API, wherein the dose is calculated to produce the desired therapeutic effect. For oral administration, the unit dosage form may be a tablet or capsule.

The pharmaceutical compositions described herein may be administered in an immediate release dosage or a modified release dosage. An immediate-release (IR) dosage includes an oral dosage form that is formulated to release the API immediately after administration. An extended-release (ER) dosage includes an oral dosage form that is formulated to release the API over a prolonged period after administration. ER dosages include sustained-release (SR) or controlled-release (CR) dosage forms. SR means the API is released over a sustained period, but not at a constant rate. CR means the API is released over a sustained period at a nearly constant rate.

Methods of defining an appropriate dose include determining the Maximum Tolerated Dose (MTD), Maximum Feasible Dose (MFD), limiting dose (1000 mg/kg), exposure saturation, or the dose providing a 50-fold margin of exposure. The MTD is the highest dose that does not cause unacceptable side effects or overt toxicity in a specific period of time, and can be determined by acute toxicity studies, short duration dose escalation studies and dose ranging studies. An appropriate dosage may also be determined by a measurable parameter, such as a reduction in tumor growth, e.g., by at least about 20%, at least about 40%, at least about 60%, or at least about 80% relative to untreated subjects. The effect may be evaluated in an animal model system predictive of efficacy in human tumors. Alternatively, the effect may be evaluated by measuring responses in vitro, ex vivo, or in vivo assays or models known to the skilled practitioner.

The dose and dosing regimen may be adjusted over time according to the individual need and the professional judgment of the person administering or supervising the administration of the compositions.

Dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan.

### Characterization of Exemplary of Heterobifunctional Compounds

Disclosed herein are heterobifunctional compounds that modulate the protein level of either CCND, or CDK4. These compounds were designed and synthesized by incorporating three moieties: DDB1 ligands, linkers, and CDK4/6 binders.

Specific exemplary heterobifunctional compounds were characterized in Calu-1, MCF7, T47D and other cells. Cells that express cyclin D1-3 and CDK4/6 proteins were treated with heterobifunctional compounds disclosed herein at indicated concentrations. MCF7 cells were deprived of serum for 2 hours followed by the treatment with heterobifunctional compounds. Cells were collected, lysed and subject to immunoblotting using an antibody specific to CCND1, CCND2, CCND3, CDK4, or FoxM1, cyclin A2, or phosphorylated Rb (S807/811) proteins. Tubulin or GAPDH was used as the loading control. DMSO treatment was used as the negative control. As illustrated in Table 6, following a 16-hour treatment of various heterobifunctional compounds at indicated concentrations, CCND1, and CDK4 protein levels in Calu-1 cells were significantly decreased.

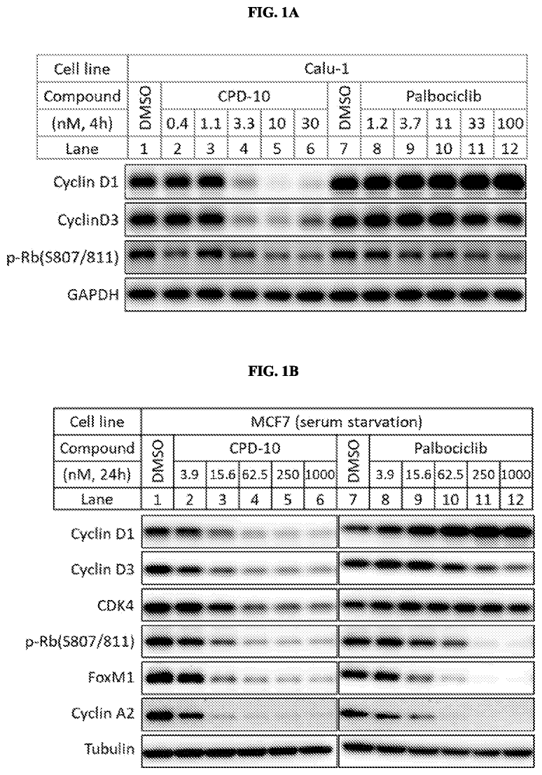

Heterobifunctional compounds, exemplified by CPD-10, were found to be particularly effective in reducing CCND1, CCND3, and CDK4 protein levels in a concentration-dependent manner in Calu-1 and MCF7 cells (FIG. 1A-1B, DC₅₀<5 nM in Calu-1, DC₅₀<20 nM in MCF7). Palbociclib, a CDK4/6 inhibitor, has no significant effect on CCND and CDK4 protein levels in Calu-1 cells, while showed accumulation of CCND1 protein levels in MCF7 cells. Heterobifunctional compounds also inhibited downstream FoxM1 and cyclin A2 protein levels, and Rb phosphorylation in a concentration-dependent manner in T47D cells (FIG. 1B).

To evaluate anti-proliferation activities of heterobifunctional compounds, NSCLC Calu-1 or ER+ breast cancer T47D cells were treated with selected heterobifunctional compounds following a 10-point serial dilution for 3 or 6 days, respectively. As illustrated in Table 7, exemplary heterobifunctional compounds potently suppressed the viability of Calu-1, T47D, or MCF7 cells.

HiBiT detection system quantitatively measures total amounts of HiBiT-tagged proteins using a luminescent based method. MCF7 cells stably expressing HiBiT tagged target proteins were treated with heterobifunctional compounds at indicated concentrations for 16 hours, then lysed with Nano-Glo HiBiT Lytic detection reagents. As illustrated in Table 8, following treatment, cyclin D1 and CDK4 protein levels were significantly decreased.

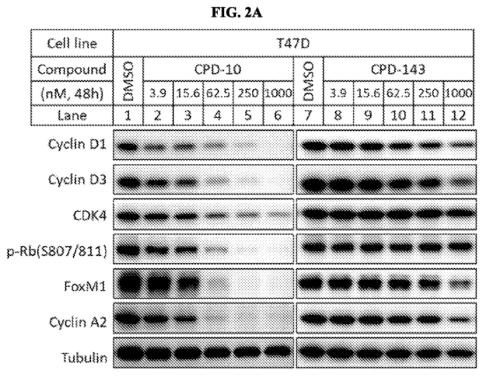

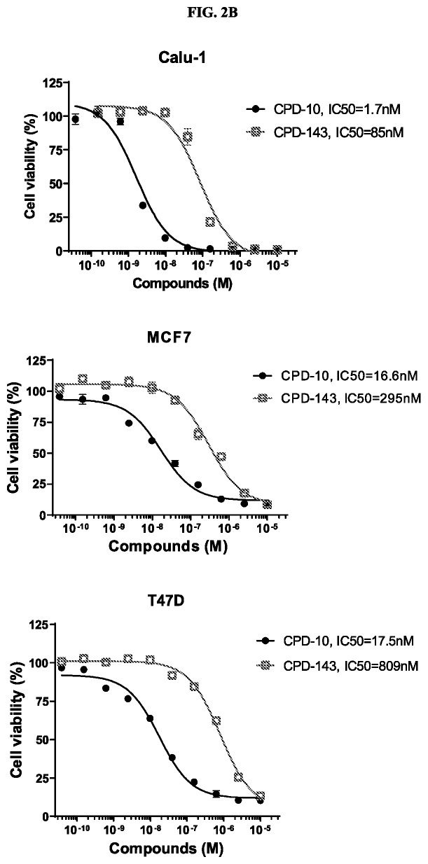

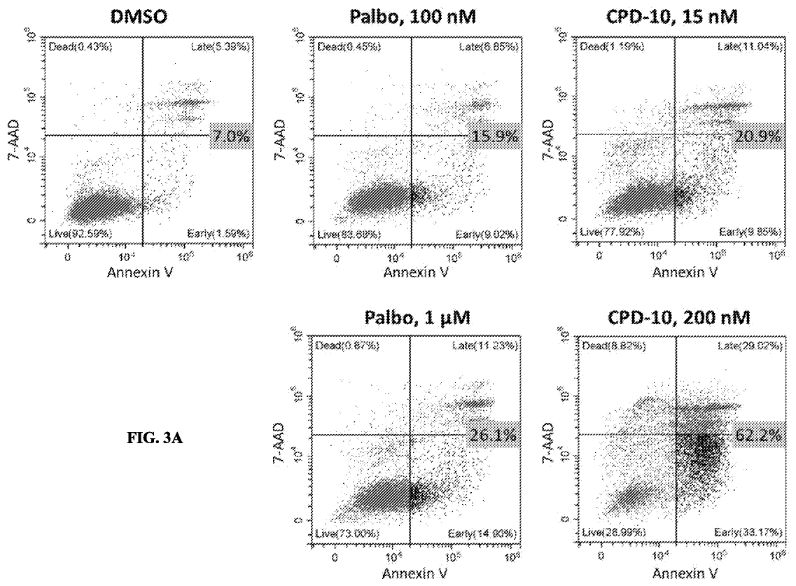

To verify that heterobifunctional compound-mediated degradation is dependent on the binding to CDK4-cyclin D complex, a negative control compound, CPD-143, which is derived from active heterobifunctional compound CPD-10 was designed. CPD-143 bears the same DDB1 ligand and linker as CPD-10 but with a modified warhead to impair the binding of the control compound to CDK4. As illustrated in FIG. 2A-2B, compared with the corresponding active heterobifunctional compound CPD-10, the negative control compound CPD-143 showed much weaker degradation potencies (FIG. 2A, >100-fold decrease in DC₅₀) and cellular anti-proliferation activities (FIG. 2B, >50-fold decrease in Calu-1 and T47D, >15-fold decrease in MCF7). These results confirm that heterobifunctional compound-mediated CCND and CDK4/6 degradation is dependent on their direct binding to CDK4.

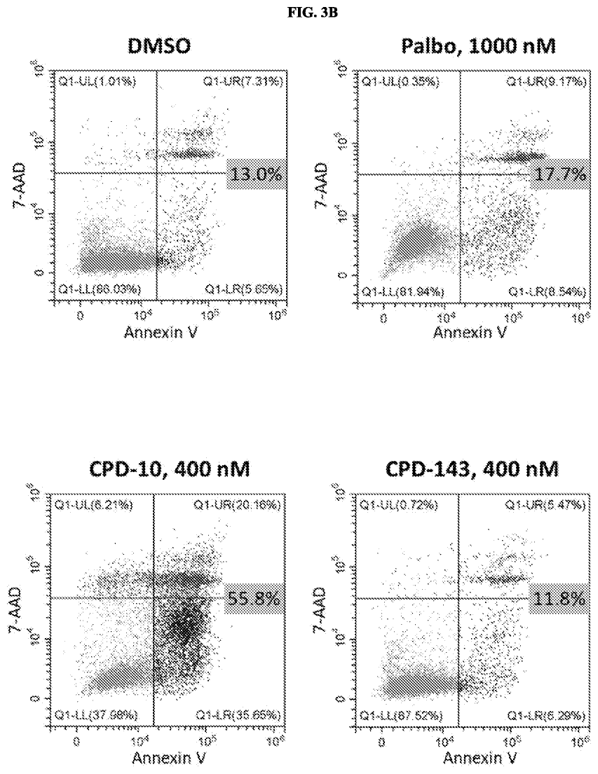

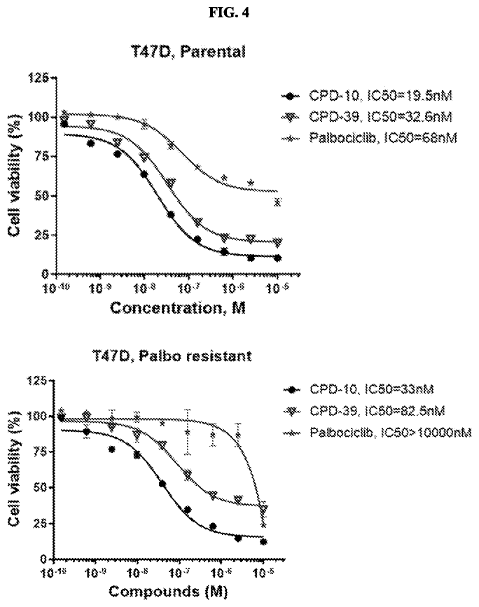

Flow cytometric analysis of Annexin V/7-AAD stained ER⁺ breast cancer cells demonstrated that heterobifunctional degraders inhibited tumor cell growth by a different mechanism of action from CDK4/6 inhibitors. T47D cells were treated with DMSO, palbociclib, heterobifunctional compound CPD-10 for 6 days at doses approximating IC₅₀and IC₉₀ concentrations as indicated (FIG. 3A). HCC1428 cells were treated with DMSO, palbociclib, heterobifunctional compound CPD-10, or its negative control compound CPD-143 for 6 days at doses approximating IC₉₀ concentrations as indicated (FIG. 3B). Cells were harvested by trypsinization, staining was carried out using the Annexin V Apoptosis Detection Kit. The percentages of early apoptotic (Annexin V⁺7-AAD⁻, lower right quadrant), late apoptotic (Annexin V⁺7-AAD⁺, upper right quadrant) and necrotic cells (Annexin V⁻7-AAD⁺, upper left quadrant) are indicated on dot plots. As illustrated in FIG. 3A-3B, heterobifunctional degrader CPD-10 was found to cause significant cell apoptosis at both doses approximating IC₅₀ and IC₉₀ concentrations in T47D and HCC1428 cells apoptosis (Annexin V⁺ population: 20.9% at 15 nM, 62.2% at 200 nM in T47D; 55.8% in HCC1428), while CDK4/6 inhibitor palbociclib or negative control compound CPD-143 showed much less effect on cell apoptosis even at the concentration up to 1 μM (palbo: 15.9% at 100 nM, 26.1% at 1 μM in T47D; palbo: 17.7% at 1 μM in HCC1428, CPD-143: 11.8% at 400 nM in HCC1428), compared to DMSO treated cells (7.0% in T47D; 13.0% in HCC1428).

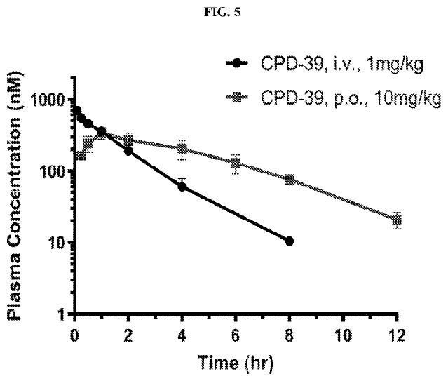

Furthermore, we developed one ER⁺ breast cancer T47D model with acquired resistance after long period of treatment with 1 μM palbociclib (over IC₉₀). Cells were deemed resistant when growing in the presence of palbo at the same rate as parental cells. Palbo resistance was determined by CellTiter-Lumi cell viability assay (FIG. 4, IC₅₀>10,000 nM). Heterobifunctional compounds CPD-10 and CPD-39 were found to remain effective in T47D palbo-resistant model compared to parental cells (FIG. 4, CPD-10: IC₅₀=19.5 nM and 33 nM in parental and palbo resistant cells; CPD-39: IC₅₀=32.6 nM and 82.5 nM in parental and palbo resistant cells). Taken together, these results indicated that degradation of CCND/CDK4 proteins could therapeutically target multiple cancer types and demonstrate more potent capability than CDK4/6 inhibitors.

To investigate the pharmacokinetics of heterobifunctional compounds, exemplified by CPD-39, a single dose of CPD-39 via 1 mg/kg intravenous (i.v.) injection or 10 mg/kg was evaluated in mice. Plasma concentration reported at each time points post dosing represents the mean value derived from 3 experimental animals. Data showed over 15% oral bioavailability of CPD-39 in fed mice (FIG. 5, AUC_{inf}, i.v.=1032.0 hr*ng/mL, AUC_{inf}, p.o.=1582.7 hr*ng/mL; CL, i.v.=16.3 mL/min/kg; V_{SS}, i.v.=1.6 L/kg).

Taken together, these results indicated that degradation of CCND proteins could therapeutically target multiple cancer types beyond breast cancer and demonstrate more potent capability than CDK4/6 inhibitors.

### Preparation of Compounds

Compounds of the invention are prepared according to the exemplary procedures and general synthetic routes provided herein, and modifications thereof using general methods known to those of skill in the art.

The compounds described herein may be made from commercially available reagents and chemical starting materials obtained from commercial sources and/or compounds described in the chemical literature. Alternatively, specific and analogous reactants can be identified and prepared through publicly available indices of known chemicals and reactions, such as the Chemical Abstract Service of the American Chemical Society, or other on-line databases. Chemicals that are known but not commercially available may optionally be prepared by custom chemical synthesis.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. A reference for the preparation and selection of pharmaceutical salts of the compound described herein is P. H. Stahl & C. G. Wermuth “Handbook of Pharmaceutical Salts”, Verlag Helvetica Chimica Acta, Zurich, 2002.

Compounds analogous to those for which experimental details are provided may be prepared according to the following General Schemes A and B, or modifications thereof known to the skilled practitioner.

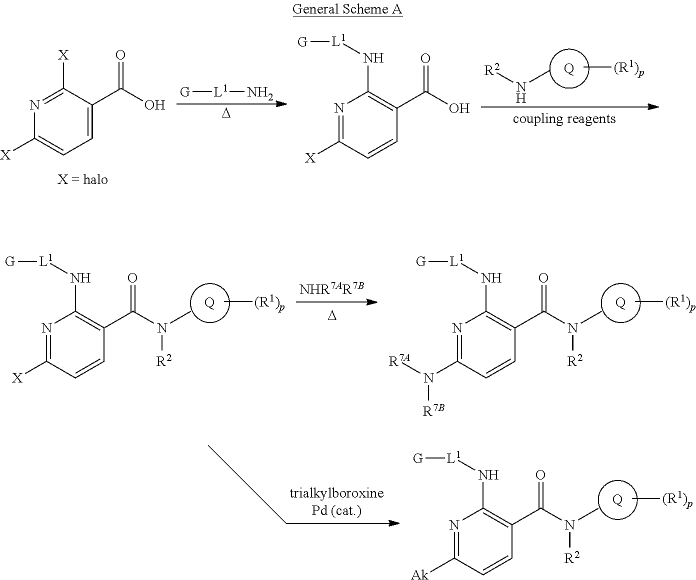

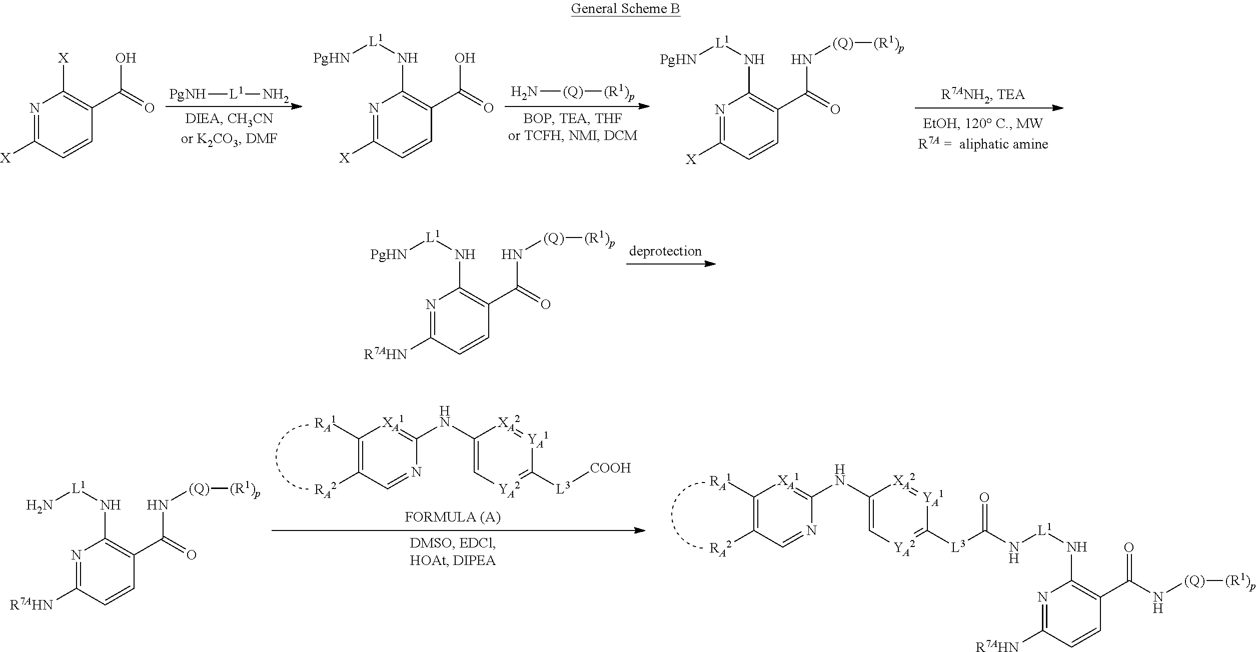

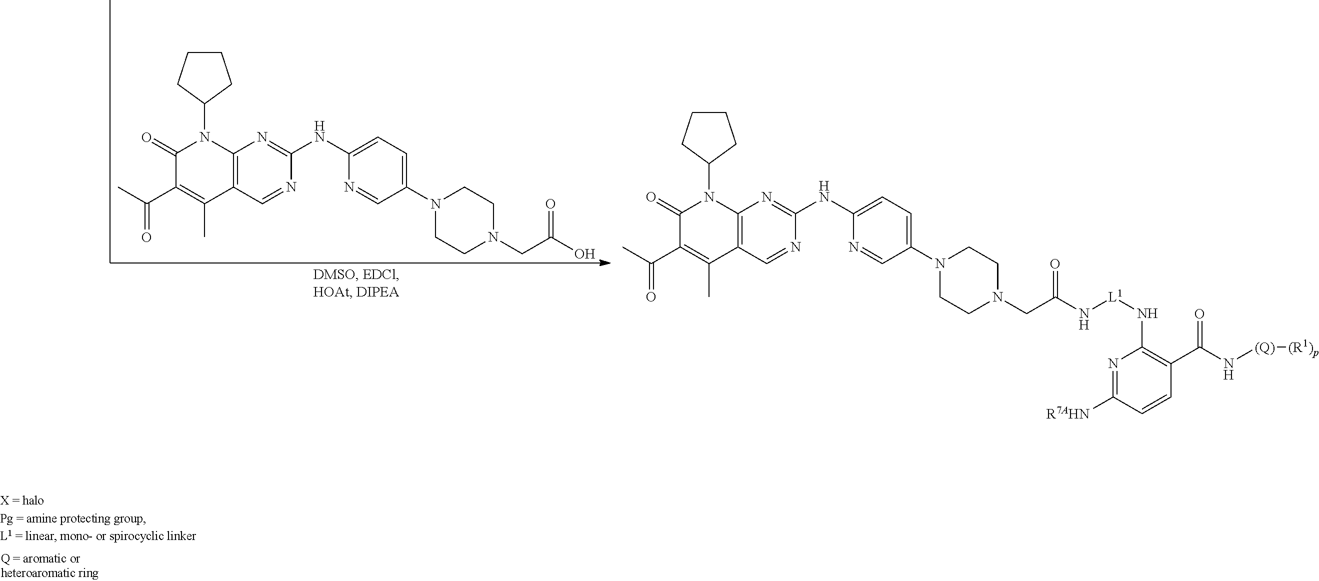

While preferred embodiments of the present invention have been shown and described herein, variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are set forth to illustrate more clearly the principle and practice of instances disclosed herein to those skilled in the art and are not to be construed as limiting the scope of any claimed instances, including specific synthetic methods of making. Unless otherwise stated, all parts and percentages are on a weight basis.

### General Chemistry Methods

All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quaternary Solvent [symbol] Manager) with degasser, an autosampler (FTN), a column oven (40 [symbol] , unless otherwise indicated), a photo-diode array PDA detector. Chromatography was performed on an AcQuity UPLC BEH C18 (1.7 mm, 2.1×50 mm) with water containing 0.10% formic acid as solvent A and acetonitrile containing 0.1% formic acid as solvent B at a flow rate of 0.6 m_{L}/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance 111400 spectrometer. Chemical shifts are expressed in parts per million (ppm) and reported as 6 value (chemical shift 6). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 μm, 30×75 mm,) at room temperature. The flow rate was 40 m_{L}/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm, or 280 nm. The flow rate was 40 mL/min. A linear gradient was used with H₂O containing 0.05% TFA as solvent A and 100% of MeOH containing 0.05% TFA as solvent B. All compounds showed 95% purity using the LCMS methods described above.

### Synthetic Methods

Compounds of the invention are prepared according to the exemplary procedures provided herein and modifications thereof known to those of skill in the art.

The following abbreviations are used throughout the Examples: “Ac” means acetyl, “AcO” or “OAc” means acetoxy, “aq.” means aqueous, “Bn” means benzyl, “BOC”, “Boc” or “boc” means N-tert-butoxycarbonyl, “BOP” means (benzotriazol-1-yloxytris(dimethylamino)phosphonium hexa-fluorophosphate), “Bu” means butyl, “nBu” means normal-butyl, “tBu” means tert-butyl, “Cbz” means benzyloxycarbonyl, “CDI” meanscarbonyldiimidazole, “DCE” (CH₂ClCH₂Cl) means 1,2-dichloroethane, “DCM” (CH₂Cl₂) means methylene chloride/dichloromethane, “DEA” means diethylamine, “DIEA” or “DIPEA” means diisopropylethylamine, “DMA” means N,N-dimethylacetamide, “DMAP” means 4-dimethylaminopyridine, “DMF” means N,N-dimethyl formamide, “DMSO” means dimethylsulfoxide, “dppa” means [azido(phenoxy)phosphoryl]-oxybenzene, “dppf” means 1,1′-bis(diphenylphosphino)ferrocene, “Et” means ethyl, “EtOAc” means ethyl acetate, “EtOH” means ethanol, “HATU” means 1-[bis(dimethylamino)methylene]-1H-1,2,3-triazolo[4,5-b]pyridinium 3-oxide hexafluorophosphate, “HOAc” or “AcOH” means acetic acid, “HOAt” means 1-hydroxy-7-azabenzotriazole, “i-Pr” or “iPr” means isopropyl, “IPA” means isopropyl alcohol, “Me” means methyl, “MeOH” means methanol, “MS” means mass spectrometry, “NMI” means 1-methylimidazole, “NMP” means N-methyl-2-pyrrolidone, “Ph” means phenyl, “PMB” means p-methoxybenzyl, “sat.” means saturated, “TBS” means tert-butyl(dimethyl)silyl, “TFCH” means N,N,N′,N′-tetramethylchloroformamidinium hexafluorophosphate, “TEA” means triethyl amine, “TFA” means trifluoroacetic acid, “THF” means tetrahydrofuran, “TLC” means thin layer chromatography, “TsCl” means p-toluenesulfonyl chloride, “rt” means room temperature, “h” means hours, “min” means minutes.

### Example 1. 2-((3-Aminopropyl)amino)-N-(4,5-dimethylthiazol-2-yl)-6-methylnicotinamide (BL1-1)

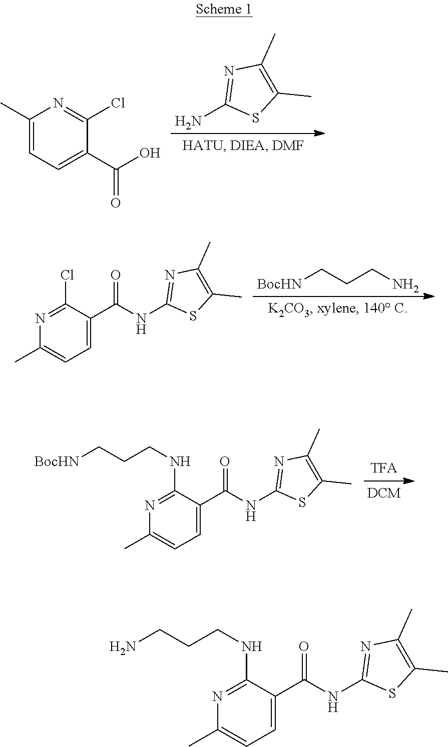

### Step 1. Synthesis of 2-chloro-N-(4,5-dimethylthiazol-2-yl)-6-methylnicotinamide

To a solution of 2-chloro-6-methylnicotinic acid (2 g, 11.7 mmol) in DMF (20 mL) were added 4,5-dimethylthiazol-2-amine (1.5 g, 11.7 mmol), HATU (4.4 g, 11.7 mmol) and DIEA (3.02 g, 23.4 mmol). The mixture was stirred at rt for 5 h, before it was concentrated under reduced pressure. The residue was purified by column chromatography on silica-gel (petroleum ether/ethyl acetate=2:1) to provide the desired product (2.07 g, 63% yield) as a white solid. MS (ESI) m/z=282.0 [M+H]⁺.

### Step 2. Synthesis of tert-butyl (3-((3-((4,5-dimethylthiazol-2-yl)carbamoyl)-6-methylpyridin-2-yl)amino)propyl)carbamate

To a solution of 2-chloro-N-(6-methoxypyridazin-3-yl)-6-methylnicotinamide (500 mg, 1.8 mmol) in xylene (10.0 mL) were added K₂CO₃ (497 mg, 3.6 mmol) and tert-butyl (3-aminopropyl)carbamate (313 mg, 1.8 mmol). The mixture was stirred at 140 [symbol] for 12 h. After cooling down to rt, the solution was concentrated and the residue was diluted with H₂O and extracted with ethyl acetate (20 ml×3). The combined organic layers were washed with brine (30 mL), dried over sodium sulfate, filtered, and concentrated. The crude product was purified by column chromatography on silica-gel (DCM/MeOH=20:1) to provide the desired product (98 mg, 13% yield) as a white solid. ¹HNMR (400 MHz, DMSO-d₆) δ 12.16 (s, 1H), 8.37 (s, 1H), 8.17 (d, J=8.0 Hz, 1H), 6.87 (t, J=5.2 Hz, 1H), 6.45 (d, J=7.6 Hz, 1H), 3.46 (q, J=6.4 Hz, 2H), 2.99 (q, J=6.4 Hz, 2H), 2.33 (s, 3H), 2.24 (s, 3H), 2.19 (s, 3H), 1.70-1.66 (m, 2H), 1.38 (s, 9H). MS (ESI) m/z=420.1 [M+H]⁺.

### Step 3. Synthesis of 2-((3-aminopropyl)amino)-N-(4,5-dimethylthiazol-2-yl)-6-methylnicotinamide

To a solution of tert-butyl (3-((3-((4,5-dimethylthiazol-2-yl)carbamoyl)-6-methylpyridin-2-yl)amino)propyl)carbamate (16 mg, 0.038 mmol) in DCM (2 mL) was added TFA (1 mL) at rt. After the reaction mixture was stirred at rt for 2 h, it was concentrated under reduced pressure to provide the desired product (18 mg, 87% yield) as TFA salt. MS (ESI) m/z=320.3 [M+H]⁺.

### Example 2. 2-((7-Aminoheptyl)amino)-N-(6-methoxypyridazin-3-yl)-6-methylnicotinamide (BL1-2)

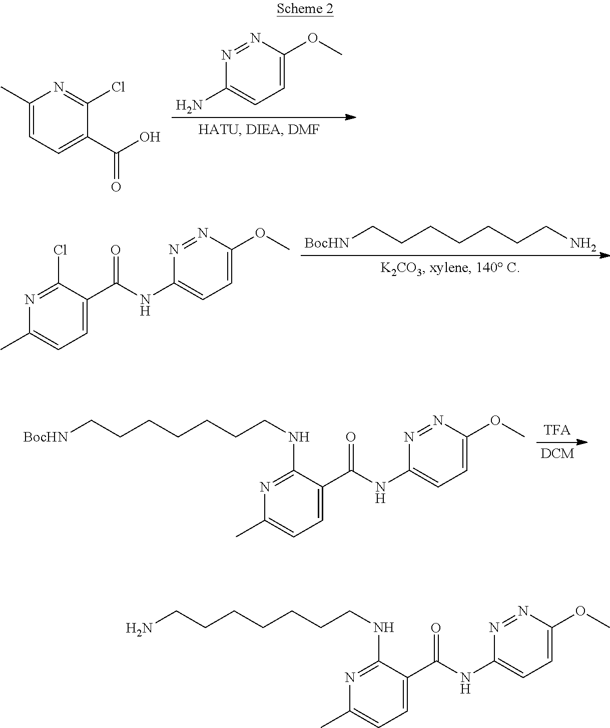

BL1-2 was synthesized following the procedures for preparing BL1-1 (15 mg, 8% yield over 3 steps) as TFA salt. MS (ESI) m/z=373.4 [M+H]⁺.

### Example 3. 2-((5-Aminopentyl)amino)-N-(4,5-dimethylthiazol-2-yl)-6-methylnicotinamide (BL1-3)

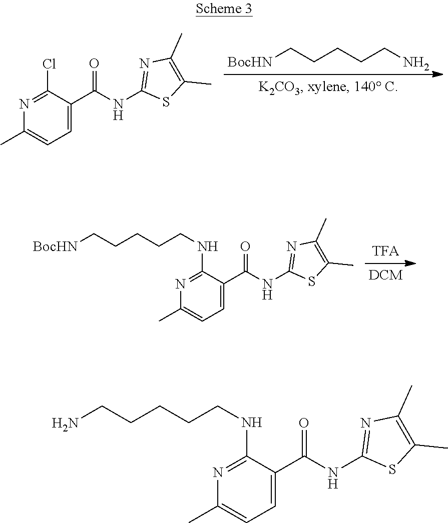

### Step 1. Synthesis of tert-butyl (5-((3-((4,5-dimethylthiazol-2-yl)carbamoyl)-6-methylpyridin-2-yl)amino)pentyl)carbamate

The title compound was synthesized following the procedure of step 2 for preparing BL1-1 (110 mg, 14% yield) as a white solid. ¹HNMR (400 MHz, DMSO-d₆) δ 12.14 (s, 1H), 8.38 (s, 1H), 8.18 (d, J=8.0 Hz, 1H), 6.76 (t, J=5.6 Hz, 1H), 6.44 (d, J=7.6 Hz, 1H), 3.42 (q, J=6.4 Hz, 2H), 2.92 (q, J=6.4 Hz, 2H), 2.33 (s, 3H), 2.24 (s, 3H), 2.18 (s, 3H), 1.60-1.53 (m, 2H), 1.44-1.39 (m, 2H), 1.36 (s, 9H), 1.33-1.29 (m, 2H). MS (ESI) m/z=448.1 [M+H]⁺.

### Step 2. Synthesis of 2-((5-aminopentyl)amino)-N-(4,5-dimethylthiazol-2-yl)-6-methylnicotinamide

The title compound was synthesized following the standard procedure to provide the desired product (13 mg, 84% yield) as TFA salt. MS (ESI) m/z=348.4 [M+H]⁺.

### Example 4. 2-((7-aminoheptyl)amino)-N-(4,5-dimethylthiazol-2-yl)-6-(methylamino)nicotinamide (BL1-4)

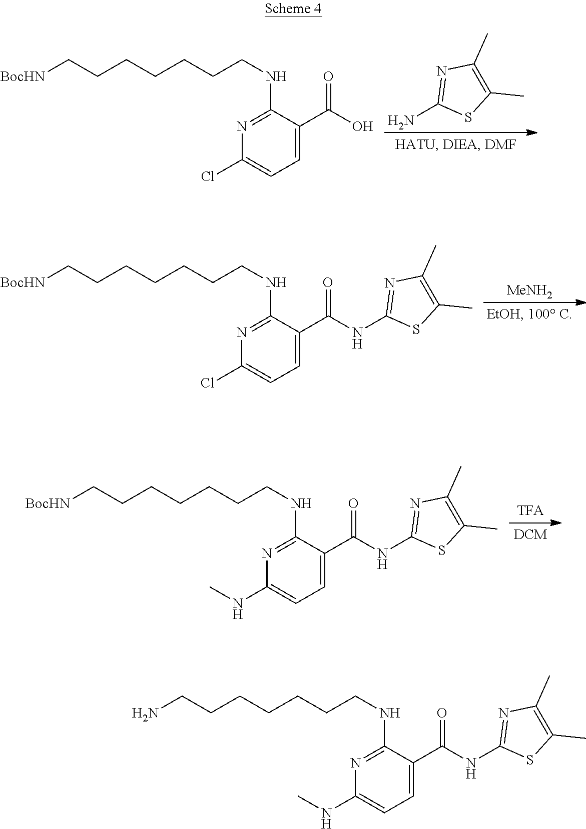

### Step 1. Synthesis of tert-butyl (7-((6-chloro-3-((4,5-dimethylthiazol-2-yl)carbamoyl)pyridin-2-yl)amino)heptyl)carbamate

A mixture of 2-((5-((tert-butoxycarbonyl)amino)pentyl)amino)-6-chloronicotinic acid (170 mg, 0.44 mmol), 4,5-dimethylthiazol-2-amine (69 mg, 0.53 mmol), HATU (201 mg, 0.53 mmol) and DIEA (170 mg, 1.32 mmol) in DMF (10.0 mL) was stirred at 40 [symbol] overnight. After cooling down to rt, the solution was concentrated under reduced pressure. The residue was purified by silica gel flash chromatography (petroleum ether/ethyl acetate=1:1) to provide the desired product (150 mg, 50% yield) as a yellow solid. MS (ESI) m/z=496.2 [M+H]⁺.

### Step 2. Synthesis of tert-butyl (7-((3-((4,5-dimethylthiazol-2-yl)carbamoyl)-6-(methylamino)pyridin-2-yl)amino)heptyl)carbamate

A mixture tert-butyl (7-((6-chloro-3-((4,5-dimethylthiazol-2-yl)carbamoyl)pyridin-2-yl)amino)heptyl)carbamate (120 mg, 0.24 mmol) and TEA (75 mg, 0.73 mmol) in methylamine (30% in ethanol, 5.0 mL) was stirred at 120 [symbol] under microwave irradiation for 2 h. After cooling down to rt, the solution was concentrated and the residue was purified by silica gel flash chromatography (petroleum ether/ethyl acetate=1:1) to provide the desired product (65 mg, 47% yield) as a yellow solid. ¹ HNMR (400 MHz, DMSO-d₆) δ 11.40 (s, 1H), 8.70-8.65 (m, 1H), 7.97 (d, J=8.4 Hz, 1H), 6.99 (s, 1H), 6.74-6.72 (m, 1H), 5.66 (d, J=8.8 Hz, 1H), 3.45-3.39 (m, 2H), 2.92-2.85 (m, 2H), 2.80 (d, J=4.8 Hz, 3H), 2.22 (s, 3H), 2.16 (s, 3H), 1.56-1.51 (m, 2H), 1.36 (s, 9H), 1.32-1.23 (m, 8H). MS (ESI) m/z=491.1 [M+H]⁺.

### Step 3. Synthesis of 2-((7-aminoheptyl)amino)-N-(4,5-dimethylthiazol-2-yl)-6-(methylamino)nicotinamide

The title compound was synthesized following the standard procedure to provide the desired product (15 mg, 97% yield) as TFA salt. MS (ESI) m/z=391.3 [M+H]⁺.

### Example 5. 2-((5-Aminopentyl)amino)-N-(6-(dimethylamino)pyridazin-3-yl)-6-(methylamino)nicotinamide (BL1-5)

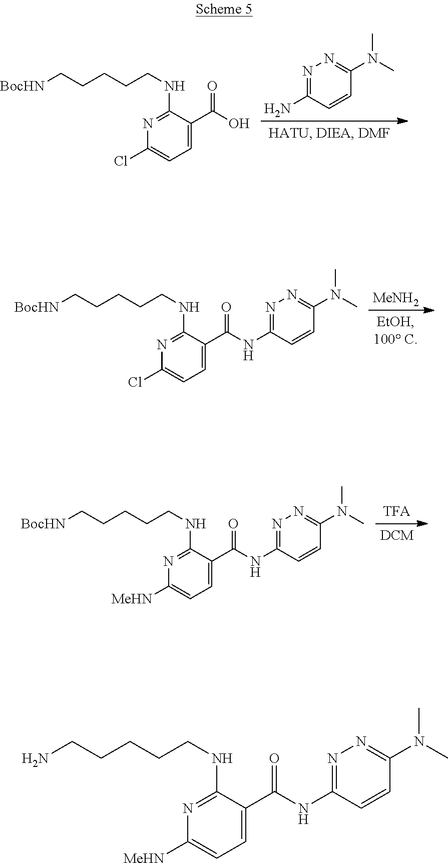

### Step 1. Synthesis of tert-butyl (5-((6-chloro-3-((6-(dimethylamino)pyridazin-3-yl)carbamoyl)pyridin-2-yl)amino)pentyl)carbamate

A mixture of 2-((5-((tert-butoxycarbonyl)amino)pentyl)amino)-6-chloronicotinic acid (300 mg, 0.78 mmol), N³,N³-dimethylpyridazine-3,6-diamine (130 mg, 0.93 mmol), HATU (380 mg, 1.0 mmol) and DIAE (300 mg, 2.34 mmol) in DMF (10.0 mL) was stirred at 40 [symbol] overnight. After cooling down to rt, the solution was concentrated under reduced pressure. The residue was purified by silica gel flash chromatography (petroleum ether/ethyl acetate=1:1) to provide the desired product (220 mg, 55% yield) as a yellow solid. MS (ESI) m/z=478.2 [M+H]⁺.

### Step 2. Synthesis of tert-butyl (5-((3-((6-(dimethylamino)pyridazin-3-yl)carbamoyl)-6-(methylamino)pyridin-2-yl)amino)pentyl)carbamate

A mixture of tert-butyl (5-((6-chloro-3-((6-(dimethylamino)pyridazin-3-yl)carbamoyl)pyridin-2-yl)amino)pentyl)carbamate (220 mg, 0.46 mmol) and TEA (140 mg, 1.38 mmol) in methylamine (30% in ethanol, 5.0 mL) was stirred at 120 [symbol] under microwave irradiation for 2 h. After cooling down to rt, the solution was concentrated under reduced pressure. The residue was purified by silica gel flash chromatography (petroleum ether/ethyl acetate=1:1) to provide the desired product (110 mg, 50% yield) as a yellow solid. ¹HNMR (400 MHz, DMSO-d₆) δ 10.04 (s, 1H), 8.72 (s, 1H), 7.95 (d, J=8.4 Hz, 1H), 7.81 (d, J=9.6 Hz, 1H), 7.12 (d, J=9.6 Hz, 1H), 6.88 (s, 1H), 6.76-6.72 (m, 1H), 5.67 (d, J=8.8 Hz, 1H), 3.42-3.36 (m, 2H), 3.07 (s, 6H), 2.93-2.88 (m, 2H), 2.81 (d, J=4.8 Hz, 3H), 1.58-1.50 (m, 2H), 1.36 (s, 9H), 1.44-1.27 (m, 4H). MS (ESI) m/z=473.2 [M+H]⁺.

### Step 3. Synthesis of 2-((5-aminopentyl)amino)-N-(6-(dimethylamino)pyridazin-3-yl)-6-(methylamino)nicotinamide

The title compound was synthesized following the standard procedure to provide the desired product (15 mg, 97% yield) as TFA salt. MS (ESI) m/z=373.4 [M+H]⁺.

### Example 6. 2-((7-Aminoheptyl)amino)-N-(6-cyclopropyl-5-methylpyridin-2-yl)-6-(methylamino)nicotinamide (BL1-6)

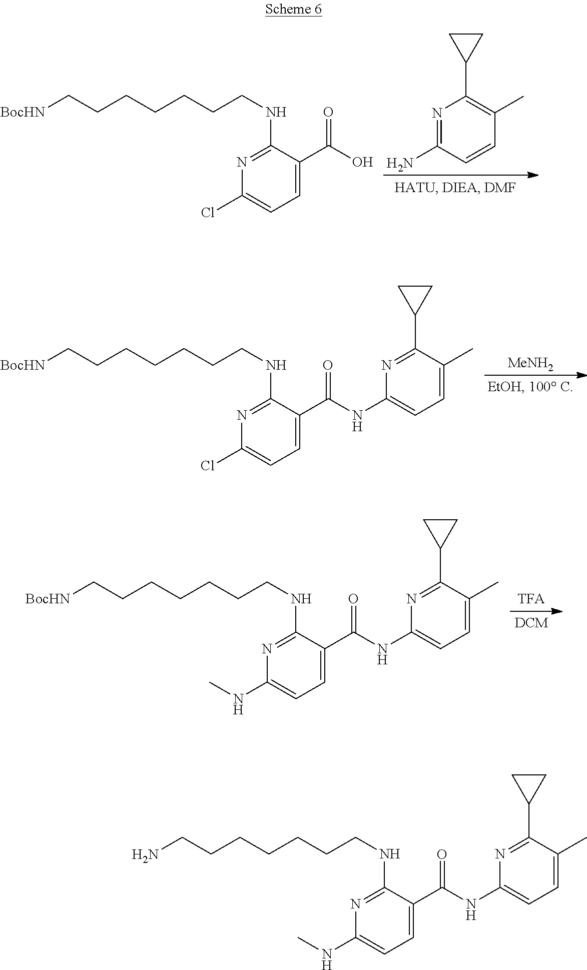 [SMILES: "MeNH2" ; CC(C)(C)OC(=O)NCCCCCCCNc1nc(Cl)ccc1C(=O)O ; Cc1ccc(N)nc1C1CC1 ; Cc1ccc(NC(=O)c2ccc(Cl)nc2NCCCCCCCNC(=O)OC(C)(C)C)nc1C1CC1 ; CNc1ccc(C(=O)Nc2ccc(C)c(C3CC3)n2)c(NCCCCCCCNC(=O)OC(C)(C)C)n1 ; CNc1ccc(C(=O)Nc2ccc(C)c(C3CC3)n2)c(NCCCCCCCN)n1]

BL1-6 was synthesized following the procedures for preparing BL1-5 (15 mg, 35% yield over 3 steps) as TFA salt. MS (ESI) m/z=411.4 [M+H]⁺.

### Example 7. 2-((5-Aminopentyl)amino)-N-(6-cyclopropyl-5-methylpyridin-2-yl)-6-(methylamino)nicotinamide (BL1-7)

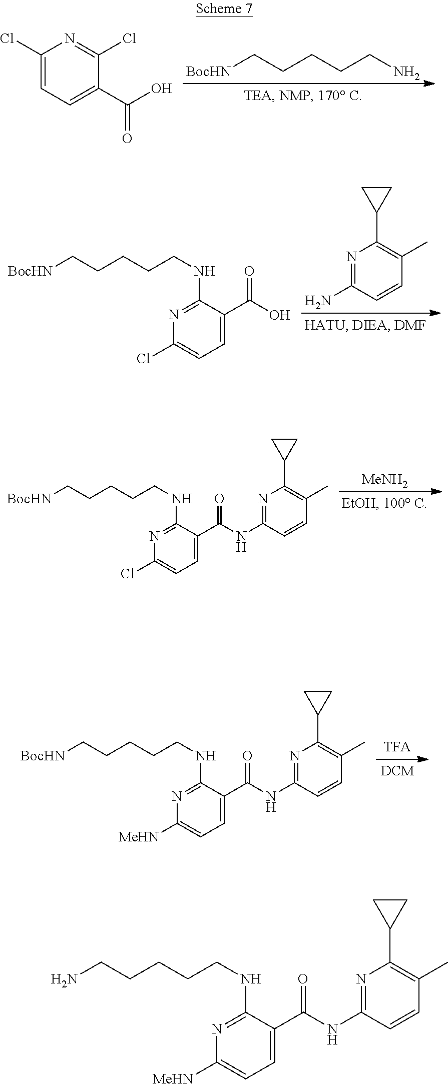

### Step 1. Synthesis of 2-((5-((tert-butoxycarbonyl)amino)pentyl)amino)-6-chloronicotinic acid

A mixture of 2,6-dichloronicotinic acid (950 mg, 4.94 mmol), tert-butyl (5-aminopentyl)carbamate (1.0 g, 4.94 mmol) and TEA (1.5 g, 14.82 mmol) in NMP (50 mL) was stirred at 170 [symbol] under microwave irradiation for 2 h. After cooling down to rt, the reaction mixture was purified by reversed-phase column chromatography to provide the title compound (800 mg, 45% yield) as a white solid. MS (ESI) m/z=358.2 [M+H]⁺.

### Step 2. Synthesis of tert-butyl (5-((6-chloro-3-((6-cyclopropyl-5-methylpyridin-2-yl)carbamoyl)pyridin-2-yl)amino)pentyl)carbamate

The title compound was synthesized following the procedure of step 1 for preparing BL1-5 to provide the desired product (320 mg, 56% yield) as a yellow solid. MS (ESI) m/z=488.4 [M+H]⁺.

### Step 3. Synthesis of tert-butyl (5-((3-((6-cyclopropyl-5-methylpyridin-2-yl)carbamoyl)-6-(methylamino)pyridin-2-yl)amino)pentyl)carbamate

The title compound was synthesized following the procedure of step 2 for preparing BL1-5 to provide the desired product (220 mg, 70% yield) as a yellow solid. ¹HNMR (400 MHz, DMSO-d₆) δ 9.31 (s, 1H), 8.72 (s, 1H), 7.88 (d, J=8.8 Hz, 1H), 7.61 (d, J=8.4 Hz, 1H), 7.42 (d, J=8.8 Hz, 1H), 6.90 (s, 1H), 6.78 (t, J=5.2 Hz, 1H), 5.67 (d, J=8.8 Hz, 1H), 3.42-3.36 (m, 2H), 2.93-2.88 (m, 2H), 2.81 (d, J=4.8 Hz, 3H), 2.32 (m, 3H), 2.12-2.05 (m, 1H), 1.58-1.50 (m, 2H), 1.36 (s, 9H), 1.44-1.30 (m, 4H), 1.01-0.97 (m, 2H), 0.90-0.86 (m, 2H). MS (ESI) m/z=483.3 [M+H]⁺.

### Step 4. Synthesis of 2-((5-aminopentyl)amino)-N-(6-cyclopropyl-5-methylpyridin-2-yl)-6-(methylamino)nicotinamide

The title compound was synthesized following the standard procedure to provide the desired product (15 mg, 97% yield) as TFA salt. MS (ESI) m/z=383.4 [M+H]⁺.

### Example 8. 2-((7-Aminoheptyl)amino)-N-(6-(dimethylamino)pyridazin-3-yl)-6-methylnicotinamide (BL1-8)

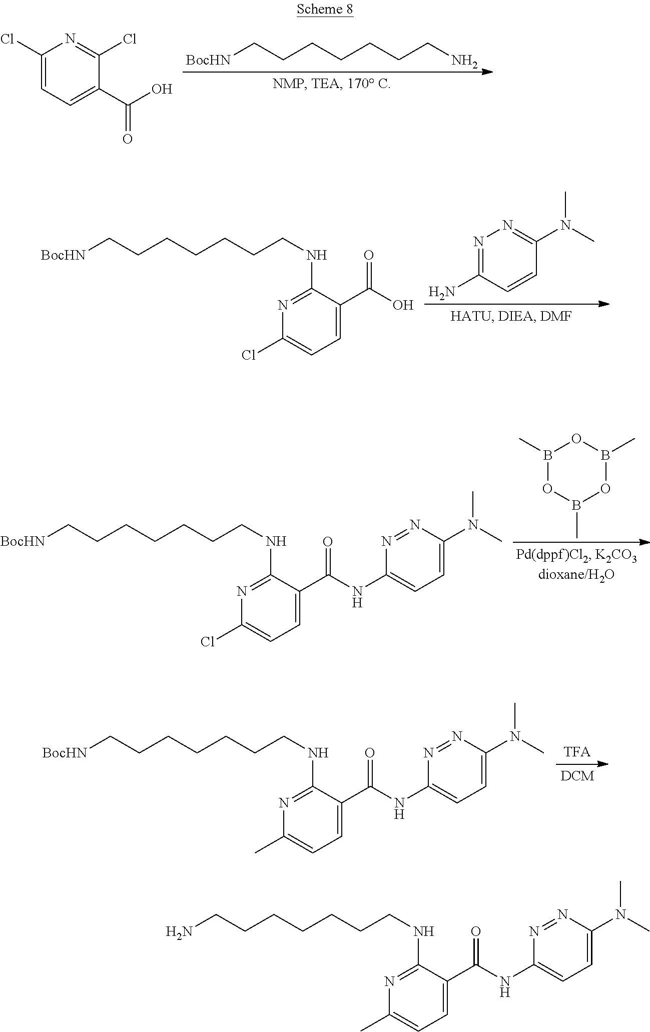

### Step 1. Synthesis of 2-((7-((tert-butoxycarbonyl)amino)heptyl)amino)-6-chloronicotinic acid

The title compound was synthesized following the procedure of step 1 for preparing BL1-7 to provide the desired product (700 mg, 45% yield) as a white solid. MS (ESI) m/z=386.2 [M+H]⁺.

### Step 2. Synthesis of tert-butyl (7-((6-chloro-3-((6-(dimethylamino)pyridazin-3-yl)carbamoyl)pyridin-2-yl)amino)heptyl)carbamate

The title compound was synthesized following the procedure of step 1 for preparing BL1-1 to provide the desired product (250 mg, 64% yield) as a yellow solid. MS (ESI) m/z=506.2 [M+H]⁺.

### Step 3. Synthesis of tert-butyl (7-((3-((6-(dimethylamino)pyridazin-3-yl)carbamoyl)-6-methylpyridin-2-yl)amino)heptyl)carbamate

A mixture of tert-butyl (7-((6-chloro-3-((6-(dimethylamino)pyridazin-3-yl)carbamoyl)pyridin-2-yl)amino)heptyl)carbamate (230 mg, 0.45 mmol), 2,4,6-trimethyl-1,3,5,2,4,6-trioxatriborinane (1.0 mL), K₂CO₃ (170 mg, 1.2 mmol) and Pd(dppf)Cl₂ in 1,4-dioxane (10.0 mL) and H₂O (2.0 mL) was heated to reflux overnight. After cooling down to rt, the reaction was quenched with water (20 mL) and extracted with ethyl acetate (30 mL×3). The combined organic layers were washed with brine, dried over anhydrous sodium sulfate, filtered, and concentrated. The residue was purified by silica gel flash chromatography to provide the desired product (170 mg, 77% yield) as a yellow solid. ¹HNMR (400 MHz, DMSO-d₆) δ 10.69 (s, 1H), 8.25 (t, J=5.2 Hz, 1H), 8.12 (d, J=7.6 Hz, 1H), 7.84 (d, J=10.0 Hz, 1H), 7.17 (d, J=10.0 Hz, 1H), 6.74 (t, J=5.4 Hz, 1H), 6.45 (d, J=8.0 Hz, 1H), 3.42-3.37 (m, 2H), 3.08 (s, 6H), 2.91-2.86 (m, 2H), 2.33 (s, 3H), 1.56-1.50 (m, 2H), 1.36 (s, 9H), 1.34-1.23 (m, 8H). MS (ESI) m/z=486.2 [M+H]⁺.

### Step 4. Synthesis of 2-((7-aminoheptyl)amino)-N-(6-(dimethylamino)pyridazin-3-yl)-6-methylnicotinamide

The title compound was synthesized following the standard procedure to provide the desired product (14 mg, 91% yield) as TFA salt. MS (ESI) m/z=386.4 [M+H]⁺.

### Example 9. 2-((7-Aminoheptyl)amino)-N-(6-cyclopropyl-5-methylpyridin-2-yl)-6-methylnicotinamide (BL1-9)

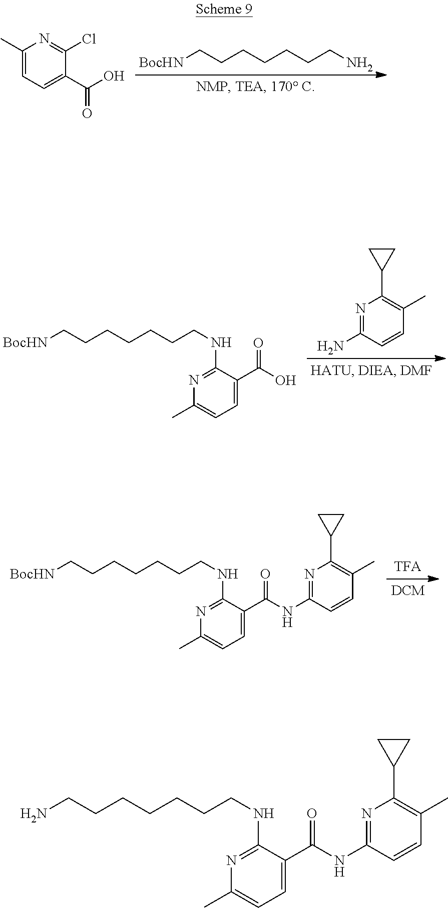 [SMILES: Cc1ccc(C(=O)O)c(Cl)n1 ; CC(C)(C)OC(=O)NCCCCCCCN ; Cc1ccc(C(=O)O)c(NCCCCCCCNC(=O)OC(C)(C)C)n1 ; Cc1ccc(N)nc1C1CC1 ; Cc1ccc(C(=O)Nc2ccc(C)c(C3CC3)n2)c(NCCCCCCCNC(=O)OC(C)(C)C)n1 ; Cc1ccc(C(=O)Nc2ccc(C)c(C3CC3)n2)c(NCCCCCCCN)n1]

### Step 1. Synthesis of 2-((7-((tert-butoxycarbonyl)amino)heptyl)amino)-6-methylnicotinic acid

The title compound was synthesized following the procedure of step 1 for preparing BL1-7 to provide the desired product (155 mg, 43% yield) as a white solid. MS (ESI) m/z=366.2 [M+H]⁺.

### Step 2. Synthesis of tert-butyl (7-((3-((6-cyclopropyl-5-methylpyridin-2-yl)carbamoyl)-6-methylpyridin-2-yl)amino)heptyl)carbamate

The title compound was synthesized following the procedure of step 1 for preparing BL1-1 to provide the desired product (55 mg, 40% yield) as a white solid. ¹HNMR (400 MHz, DMSO-d6) δ 10.04 (s, 1H), 8.15 (t, J=5.2 Hz, 1H), 8.04 (d, J=7.6 Hz, 1H), 7.64 (d, J=8.4 Hz, 1H), 7.49 (d, J=8.4 Hz, 1H), 6.74 (t, J=5.4 Hz, 1H), 6.44 (d, J=7.6 Hz, 1H), 3.42-3.37 (m, 2H), 2.91-2.85 (m, 2H), 3.35 (s, 3H), 2.33 (s, 3H), 2.13-2.09 (m, 1H), 1.56-1.52 (m, 2H), 1.36 (s, 9H), 1.32-1.22 (m, 8H), 1.00-0.97 (m, 2H), 0.92-0.88 (m, 2H). MS (ESI) m/z=496.2 [M+H]⁺.

### Step 3. Synthesis of 2-((7-aminoheptyl)amino)-N-(6-cyclopropyl-5-methylpyridin-2-yl)-6-methylnicotinamide

The title compound was synthesized following the standard procedure to provide the desired product (10 mg, 97% yield) as TFA salt. MS (ESI) m/z=396.4 [M+H]⁺.

### Example 10. 2-((7-Aminoheptyl)amino)-N-(6-(dimethylamino)pyridazin-3-yl)-6-(methylamino)nicotinamide (BL1-10)

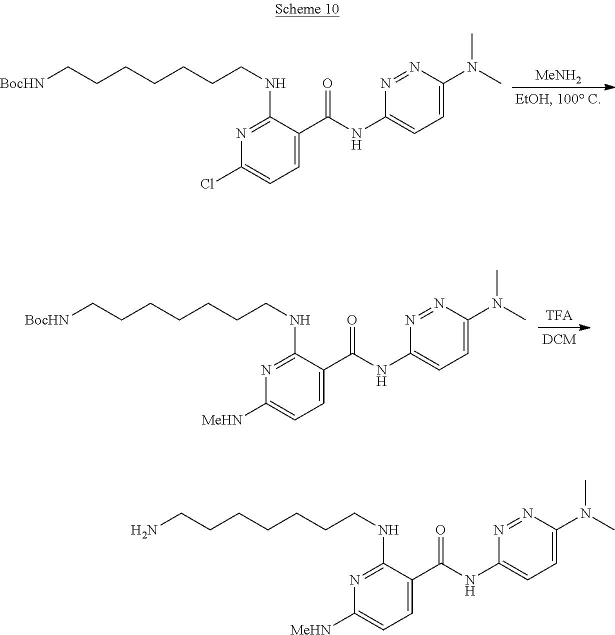 [SMILES: "MeNH2" ; CN(C)c1ccc(NC(=O)c2ccc(Cl)nc2NCCCCCCCNC(=O)OC(C)(C)C)nn1 ; CNc1ccc(C(=O)Nc2ccc(N(C)C)nn2)c(NCCCCCCCNC(=O)OC(C)(C)C)n1 ; CNc1ccc(C(=O)Nc2ccc(N(C)C)nn2)c(NCCCCCCCN)n1]

### Step 1. Synthesis of tert-butyl (7-((3-((6-(dimethylamino)pyridazin-3-yl)carbamoyl)-6-(methylamino)pyridin-2-yl)amino)heptyl)carbamate

The title compound was synthesized following the procedure of step 2 for preparing BL1-5 to provide the desired product (90 mg, 45% yield) as a yellow solid. ¹HNMR (400 MHz, DMSO-d₆) δ 10.03 (s, 1H), 8.73 (s, 1H), 7.95 (d, J=8.8 Hz, 1H), 7.80 (d, J=8.8 Hz, 1H), 7.13 (d, J=9.6 Hz, 1H), 6.88 (s, 1H), 6.73 (t, J=5.4 Hz, 1H), 5.67 (d, J=8.4 Hz, 1H), 3.43-3.36 (m, 2H), 3.07 (s, 6H), 2.90-2.85 (m, 2H), 2.81 (d, J=4.8 Hz, 3H), 1.56-1.50 (m, 2H), 1.36 (s, 9H), 1.34-1.24 (m, 8H). MS (ESI) m/z=501.2 [M+H]⁺.

### Step 2. Synthesis of 2-((7-aminoheptyl)amino)-N-(6-(dimethylamino)pyridazin-3-yl)-6-(methylamino)nicotinamide

The title compound was synthesized following the standard procedure to provide the desired product (61 mg, 98% yield) as TFA salt. MS (ESI) m/z=401.6 [M+H]⁺.

### Example 11. 2-((7-Aminoheptyl)amino)-N-(6-methoxypyridazin-3-yl)-6-(methylamino)nicotinamide (BL1-11)

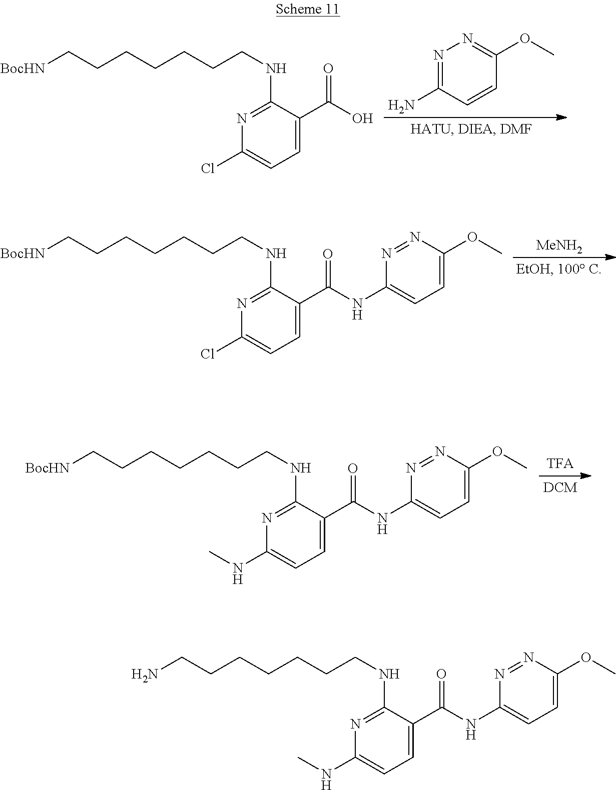

### Step 1. Synthesis of tert-butyl (7-((6-chloro-3-((6-methoxypyridazin-3-yl)carbamoyl)pyridin-2-yl)amino)heptyl)carbamate

The title compound was synthesized following the procedure of step 1 for preparing BL1-5 to provide the desired product (190 mg, 35% yield) as a yellow solid. MS (ESI) m/z=493.2 [M+H]⁺.

### Step 2. Synthesis of tert-butyl (7-((3-((6-methoxypyridazin-3-yl)carbamoyl)-6-(methylamino)pyridin-2-yl)amino)heptyl)carbamate

The title compound was synthesized following the procedure of step 2 for preparing BL1-5 to provide the desired product (150 mg, 84% yield) as a yellow solid. ¹HNMR (400 MHz, DMSO-d₆) 10.31 (s, 1H), 8.70 (brs, 1H), 8.05 (d, J=9.6 Hz, 1H), 7.96 (d, J=8.8 Hz, 1H), 7.19 (d, J=9.2 Hz, 1H), 6.96 (s, 1H), 6.73 (s, 1H), 5.68 (d, J=8.8 Hz, 1H), 3.99 (s, 3H), 3.43-3.38 (m, 2H), 2.91-2.85 (m, 2H), 2.81 (d, J=4.8 Hz, 3H), 1.56-1.53 (m, 2H), 1.36 (s, 9H), 1.34-1.20 (m, 8H). MS (ESI) m/z=488.2 [M+H]⁺.

### Step 3. Synthesis of 2-((7-aminoheptyl)amino)-N-(6-methoxypyridazin-3-yl)-6-(methylamino)nicotinamide

The title compound was synthesized following the standard procedure to provide the desired product (12 mg, 97% yield) as TFA salt. MS (ESI) m/z=388.4 [M+H]⁺.

### Example 12. 6-((7-Aminoheptyl)amino)-6′-cyano-N-(4,5-dimethylthiazol-2-yl)-[2,3′-b]pyridine]-5-carboxamide (BL1-12)

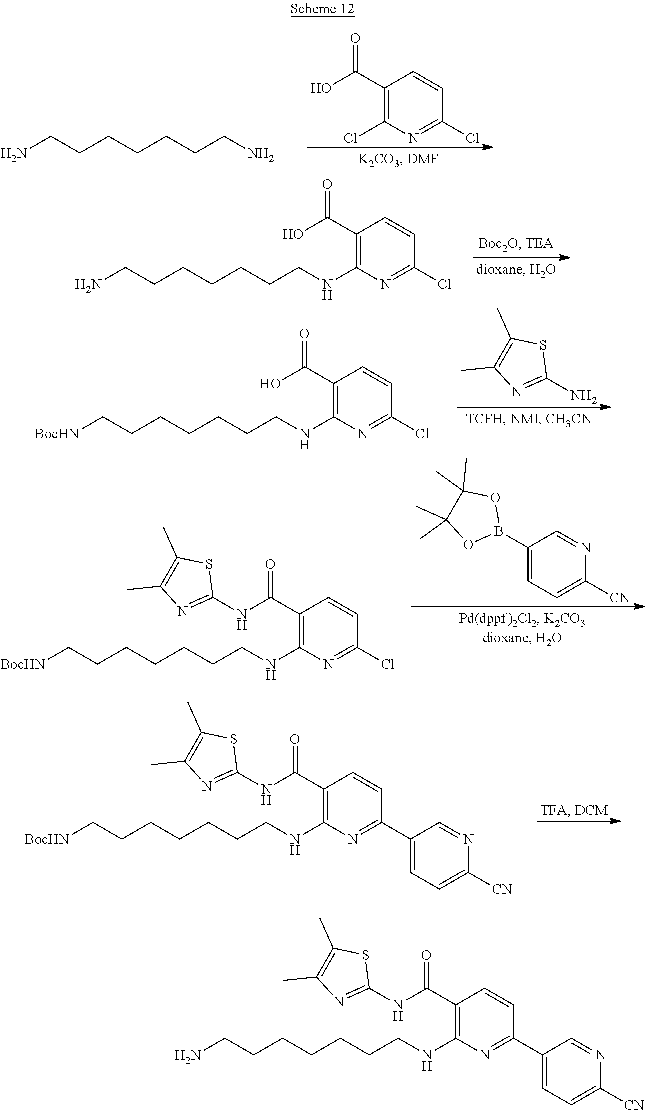

### Step 1. Synthesis of 2-((7-amino heptyl)amino)-6-chloronicotinic acid

To a solution of 2,6-dichloropyridine-3-carboxylic acid (500 mg, 2.6 mmol) in DMSO (6 mL) were added heptane-1,7-diamine (678.3 mg, 5.2 mmol) and K₂CO₃ (467.9 mg, 3.4 mmol). The mixture was stirred at 100° C. for 1 h, before it was purified by reverse-phase chromatography (H₂O/MeOH=100:0 to 60:40) to provide the title compound (400 mg, 53% yield). MS (ESI) m/z=286.09 [M+H]⁺.

### Step 2. Synthesis of 2-((7-((tert-butoxycarbonyl)amino)heptyl)amino)-6-chloronicotinic acid

To a solution of 2-(7-aminoheptylamino)-6-chloro-pyridine-3-carboxylic acid (400 mg, 1.4 mmol) in dioxane (5 mL) and water (5 mL) were added Boc₂O (336.0 mg, 1.54 mmol) and TEA (155.8 mg, 1.54 mmol). The mixture was stirred at 25° C. for 1 h, before it was purified by reverse-phase chromatography (H₂O/MeOH=100:0 to 60:40) to provide the title compound (500 mg, 92% yield). MS (ESI) m/z=286.10 [M+H-100]⁺.

### Step 3. Synthesis of tert-butyl (7-((6-chloro-3-((4,5-dimethylthiazol-2-yl)carbamoyl)pyridin-2-yl)amino)heptyl)carbamate

To a solution of 2-((7-((tert-butoxycarbonyl)amino)heptyl)amino)-6-chloronicotinic acid (300 mg, 777.43 mmol), 4,5-dimethylthiazol-2-amine (129.56 mg, 1.01 mmol) and 1-methylimidazole (NMI) (226.14 mg, 2.72 mmol) in CH₃CN (6 mL) was added TCFH (33.62 mg, 932.92 mmol). The mixture was stirred at rt for 6 h, before it was concentrated under reduced pressure and purified by reverse-phase chromatography (water/CH₃CN=100:0 to 0:100) and prep-TLC (DCM/MeOH=20:1) to provide the title compound (143 mg, 36% yield). MS (ESI) m/z=496.18 [M+H]⁺.

### Step 4. Synthesis of tert-butyl (7-((6′-cyano-5-((4,5-dimethylthiazol-2-yl)carbamoyl)-[2,3′-bipyridin]-6-yl)amino)heptyl)carbamate

To a solution of tert-butyl (7-((6-chloro-3-((4,5-dimethylthiazol-2-yl)carbamoyl)pyridin-2-yl)amino)heptyl)carbamate (60.00 mg, 120.95 mmol) in dioxane (2 mL) and water (0.4 mL) were added K₂CO₃ (50.15 mg, 362.86 mmol) and 5-(4,4,5,5-tetramethyl-1,3,2-dioxaborolan-2-yl)pyridine-2-carbonitrile (36.18 mg, 157.24 mmol). The mixture was degassed and purged with N₂ for three times, and Pd(dppf)₂Cl₂ (8.78 mg, 12.10 mmol) was added. The mixture was stirred at 80° C. for 2 h, before it was filtered and concentrated. The residue was purified by reverse-phase chromatography (water/CH₃CN=100:0 to 48:52) to provide the title compound (54 mg, 75% yield). MS (ESI) m/z=564.25 [M+H]⁺.

### Step 5. Synthesis of 6-((7-aminoheptyl)amino)-6′-cyano-N-(4,5-dimethylthiazol-2-yl)-[2,3′-bipyridine]-5-carboxamide

To a solution of tert-butyl (7-((6′-cyano-5-((4,5-dimethylthiazol-2-yl)carbamoyl)-[2,3′-bipyridin]-6-yl)amino)heptyl)carbamate (25 mg, 44.35 mmol) in DCM (3 mL) was add TFA (1.5 mL). The mixture was stirred at rt for 0.5 h, before it was concentrated to provide the title compound (TFA salt, 25 mg, 96% yield).

### Example 13. 2-((5-Aminopentyl)amino)-6-(methylamino)-N-(6-(pyrrolidin-1-yl)pyridazin-3-yl)nicotinamide (BL1-13)

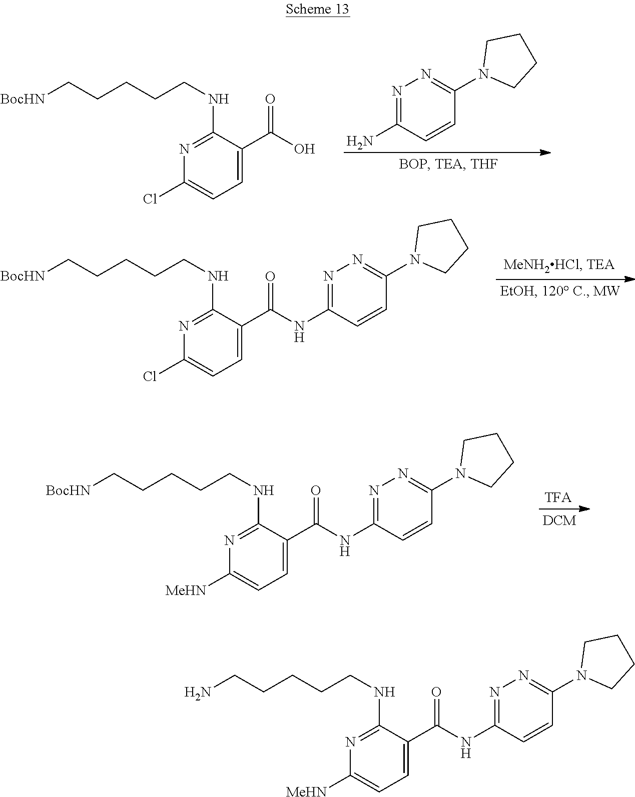 [SMILES: CC(C)(C)OC(=O)NCCCCCNc1nc(Cl)ccc1C(=O)O ; Nc1ccc(N2CCCC2)nn1 ; CC(C)(C)OC(=O)NCCCCCNc1nc(Cl)ccc1C(=O)Nc1ccc(N2CCCC2)nn1 ; CNc1ccc(C(=O)Nc2ccc(N3CCCC3)nn2)c(NCCCCCNC(=O)OC(C)(C)C)n1 ; CNc1ccc(C(=O)Nc2ccc(N3CCCC3)nn2)c(NCCCCCN)n1]

### Step 1. Synthesis of tert-butyl (5-((6-chloro-3-((6-(pyrrolidin-1-yl)pyridazin-3-yl)carbamoyl)pyridin-2-yl)amino)pentyl)carbamate

To a solution of 2-[5-(tert-butoxycarbonylamino)pentylamino]-6-chloro-pyridine-3-carboxylic acid (300 mg, 0.84 mmol), BOP (450 mg, 1.02 mmol) and TEA (262 mg, 2.59 mmol) in THF (3.0 mL) was added 6-pyrrolidin-1-ylpyridazin-3-amine (150 mg, 0.91 mmol). After stirring at rt for 12 h, the mixture was poured into water (30.0 mL) and extracted with ethyl acetate (15.0 mL×2). The combined organic phase was washed with brine (30.0 mL), dried with anhydrous Na₂SO₄, filtered, and concentrated in vacuum. The residue was purified by prep-TLC (petroleum ether/ethyl acetate=1:2) to provide the desired product (161 mg, 38% yield) as yellow solid. MS (ESI) m/z=504.3 [M+H]⁺.

### Step 2. Synthesis of tert-butyl (5-((6-(methylamino)-3-((6-(pyrrolidin-1-yl)pyridazin-3-yl)carbamoyl)pyridin-2-yl)amino)pentyl)carbamate

To a solution of tert-butyl N-[5-[[6-chloro-3-[(6-pyrrolidin-1-ylpyridazin-3-yl)carbamoyl]-2-pyridyl]amino]pentyl]carbamate (161 mg, 0.32 mmol) and TEA (218 mg, 2.16 mmol) in EtOH (2.00 mL) was added methanamine hydrochloride (608 mg, 9.0 mmol). The reaction mixture was stirred at 120° C. for 2 h under microwave irradiation. After cooling down to rt, the mixture was poured into water (20.0 mL) and extracted with EtOAc (5.0 mL×3). The combined organic phase was washed with brine (10.0 mL), dried with anhydrous Na₂SO₄, filtered, and concentrated in vacuum. The resulting residue was purified by prep-TLC (petroleum ether/ethyl acetate=0:1) to provide desired product (86.8 mg, 53% yield) as a yellow solid. ¹HNMR (400 MHz, MeOH-d₄) δ 7.99 (d, J=9.6 Hz, 1H), 7.76 (d, J=8.8 Hz, 1H), 7.00 (d, J=9.6 Hz, 1H), 6.53 (s, 0.5H), 5.74 (d, J=8.8 Hz, 1H), 3.50 (t, J=6.4 Hz, 6H), 3.05 (q, J=6.8 Hz, 2H), 2.91 (s, 3H), 2.09-2.04 (m, 4H), 1.70-1.60 (m, 2H), 1.57-1.48 (m, 2H), 1.46-1.43 (m, 2H), 1.42 (s, 9H). MS (ESI) m/z=499.5 [M+H]⁺.

### Step 3. Synthesis of 2-((5-aminopentyl)amino)-6-(methylamino)-N-(6-(pyrrolidin-1-yl)pyridazin-3-yl)nicotinamide

The title compound was synthesized following the standard procedure to provide the desired product (11 mg, 97% yield) as TFA salt. MS (ESI) n/z=399.5 [M+H]⁺.

### Example 14. 2-((5-Aminopentyl)amino)-N-(6-(dimethylamino)pyridazin-3-yl)-6-(isopropylamino)nicotinamide (BL1-14)

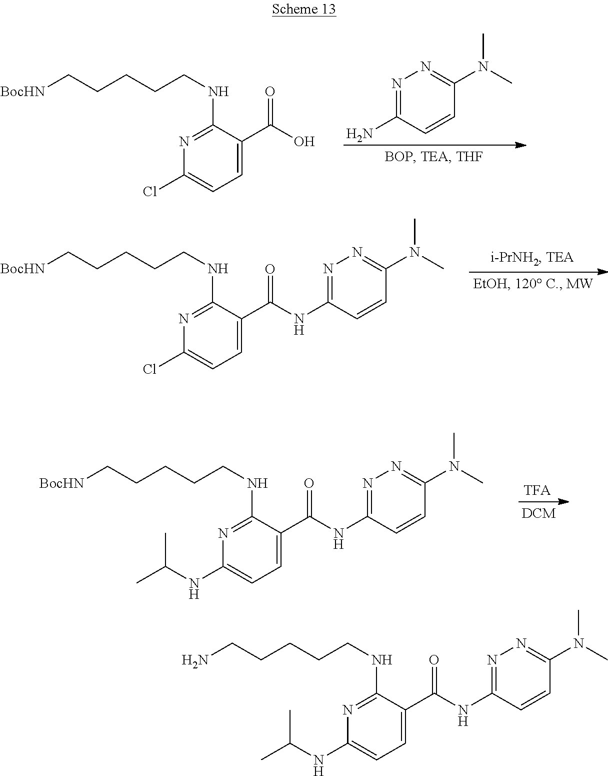

### Step 1. Synthesis of tert-butyl (5-((6-chloro-3-((6-(dimethylamino)pyridazin-3-yl)carbamoyl)pyridin-2-yl)amino)pentyl)carbamate

The title compound was synthesized following the procedure of step 1 for preparing BL1-13 to provide the desired product (287 mg, 72% yield) as a yellow solid. MS (ESI) m/z=478.2 [M+H]⁺.

### Step 2. Synthesis of tert-butyl (5-((3-((6-(dimethylamino)pyridazin-3-yl)carbamoyl)-6-(isopropylamino)pyridin-2-yl)amino)pentyl)carbamate

The title compound was synthesized following the procedure of step 2 for preparing BL1-13 to provide the desired product (95 mg, 30% yield) as a yellow solid. ¹HNMR (400 MHz, MeOH-d₄) δ 8.00 (d, J=9.6 Hz, 1H), 7.73 (d, J=8.4 Hz, 1H), 7.18 (d, J=9.6 Hz, 1H), 6.61-6.51 (m, 1H), 5.73 (d, J=9.2 Hz, 1H), 4.23-4.09 (m, 1H), 3.48 (t, J=6.8 Hz, 2H), 3.13 (s, 6H), 3.08-2.97 (m, 2H), 1.65 (q, J=7.2 Hz, 2H), 1.56-1.47 (m, 2H), 1.42 (s, 11H), 1.22 (d, J=6.8 Hz, 6H). MS (ESI) m/z=501.4 [M+H]⁺.

### Step 3. Synthesis of 2-((5-aminopentyl)amino)-N-(6-(dimethylamino)pyridazin-3-yl)-6-(isopropylamino)nicotinamide

The title compound was synthesized following the standard procedure to provide the desired product (15 mg, 97% yield) as TFA salt. MS (ESI) m/z=401.5 [M+H]⁺.

### Example 15. 2-((5-Aminopentyl)amino)-N-(6-(dimethylamino)pyridazin-3-yl)-6-methylnicotinamide (BL1-15)

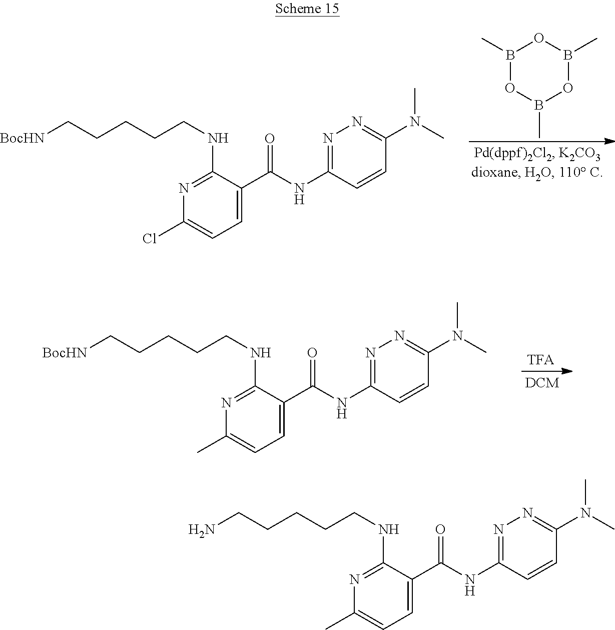

### Step 1. Synthesis of tert-butyl (5-((3-((6-(dimethylamino)pyridazin-3-yl)carbamoyl)-6-methylpyridin-2-yl)amino)pentyl)carbamate

To a solution of tert-butyl N-[5-[[6-chloro-3-[[6-(dimethylamino)pyridazin-3-yl]carbamoyl]-2-pyridyl]amino]pentyl]carbamate (232 mg, 0.49 mmol) and 2,4,6-trimethyl-1,3,5,2,4,6-trioxatriborinane (898 mg, 3.58 mmol) in dioxane (4 mL) and H₂O (1 mL) were added K₂CO₃ (203 mg, 1.47 mmol) and Pd(dppf)Cl₂ (36 mg, 0.049 mmol). The reaction mixture was stirred at 110° C. for 4 h. After cooling down to rt, the reaction mixture was concentrated under reduced pressure. The residue was purified by column chromatography on silica gel (petroleum ether/ethyl acetate=5:1 to 0:1) to provide the desired product (111 mg, 41% yield) as a yellow gum. ¹HNMR (400 MHz, MeOH-d₄) δ 8.06 (d, J=10.0 Hz, 1H), 7.99 (d, J=7.6 Hz, 1H), 7.22 (d, J=10.0 Hz, 1H), 6.50 (d, J=8.0 Hz, 1H), 3.51 (t, J=6.8 Hz, 2H), 3.17 (s, 6H), 3.10-3.02 (m, 3H), 1.75-1.64 (m, 2H), 1.59-1.51 (m, 2H), 1.50-1.45 (m, 2H), 1.43 (s, 9H).

### Step 2. Synthesis of 2-((5-aminopentyl)amino)-N-(6-(dimethylamino)pyridazin-3-yl)-6-methylnicotinamide

The title compound was synthesized following the standard procedure to provide the desired product (11 mg, 97% yield) as TFA salt. MS (ESI) m/z=358.4 [M+H]⁺.

### Example 16. 2-((5-Aminopentyl)amino)-6-cyclopropyl-N-(6-(dimethylamino)pyridazin-3-yl)nicotinamide (BL1-16)

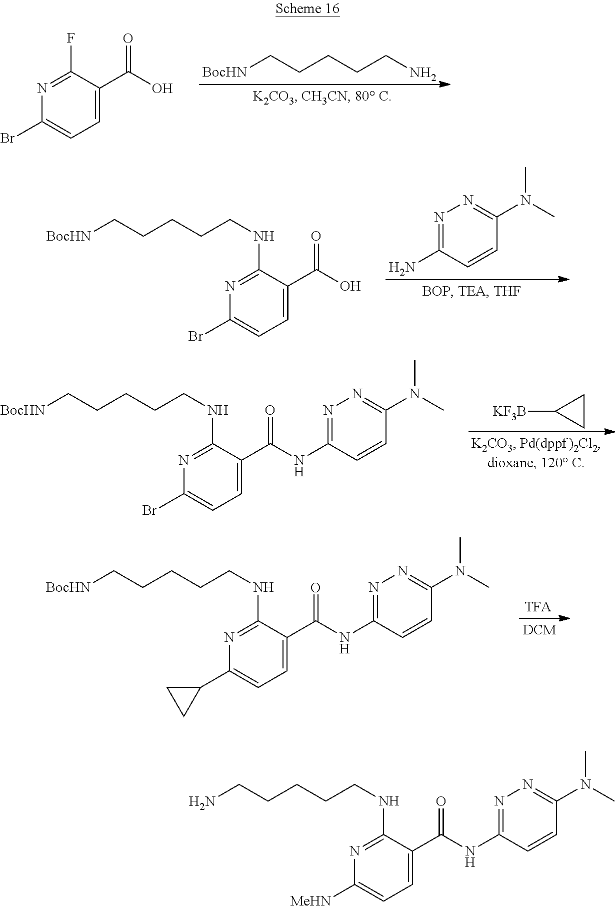

### Step 1. Synthesis of 6-bromo-2-((5-((tert-butoxycarbonyl)amino)pentyl)amino)nicotinic acid

To a solution of 6-bromo-2-fluoro-pyridine-3-carboxylic acid (2.00 g, 9.09 mmol) and tert-butyl N-(5-aminopentyl)carbamate (1.84 g, 9.10 mmol) in CH₃CN (30.0 mL) was added K₂CO₃ (2.51 g, 18.2 mmol) at rt. The reaction mixture was stirred at 80° C. for 12 h, before it was filtered and the filtrate was concentrated under reduced pressure. The residue was purified by silica gel column chromatography (petroleum ether/ethyl acetate=20:1 to 5:1) to provide the desired product (3.80 g, 97% yield) as a yellow solid. ¹HNMR (400 MHz, DMSO-d6) δ 9.50 (s, 1H), 7.83 (d, J=8.0 Hz, 1H), 6.77 (t, J=5.2 Hz, 1H), 6.55 (d, J=7.6 Hz, 1H), 3.30-3.27 (m, 2H), 2.90 (q, J=6.8 Hz, 2H), 1.54-1.48 (m, 2H), 1.42-1.37 (m, 2H), 1.36 (s, 9H), 1.34-1.29 (m, 2H).

### Step 2. Synthesis of tert-butyl (5-((6-bromo-3-((6-(dimethylamino)pyridazin-3-yl)carbamoyl)pyridin-2-yl)amino)pentyl)carbamate

To a solution of 6-bromo-2-[5-(tert-butoxycarbonylamino)pentylamino]pyridine-3-carboxylic acid (3.70 g, 8.74 mmol) in THF (40 mL) were added BOP (3.86 g, 8.74 mmol), TEA (2.65 g, 26.2 mmol, 3.65 mL) and N³,N³-dimethylpyridazine-3,6-diamine (1.20 g, 8.65 mmol). The reaction mixture was stirred at rt for 6 h, before it was poured into water (50 mL) and extracted with ethyl acetate (20 mL×3). The combined organic layers were washed with brine (60 mL), dried over Na₂SO₄, filtered, and concentrated. The residue was purified by silica gel column chromatography (petroleum ether/ethyl acetate=5:1 to 1:2) to provide the desired product (1.80 g, 39% yield) as a yellow solid. ¹HNMR (400 MHz, MeOH-d₄) δ 8.04 (d, J=9.6 Hz, 1H), 7.91 (d, J=8.0 Hz, 1H), 7.20 (d, J=10.0 Hz, 1H), 6.73 (d, J=8.0 Hz, 1H), 6.57 (s, 1H), 3.46 (t, J=7.2 Hz, 2H), 3.15 (s, 6H), 3.05 (q, J=6.8 Hz, 2H), 1.69-1.60 (m, 2H), 1.57-1.49 (m, 2H), 1.41 (s, 11H).

### Step 3. Synthesis of tert-butyl (5-((6-cyclopropyl-3-((6-(dimethylamino)pyridazin-3-yl)carbamoyl)pyridin-2-yl)amino)pentyl)carbamate

To the solution of tert-butyl N-[5-[[6-bromo-3-[[6-(dimethylamino)pyridazin-3-yl]carbamoyl]-2-pyridyl]amino]pentyl]carbamate (500 mg, 0.96 mmol) in dioxane (20 mL) and H₂O (6 mL) were added cyclopropyl(trifluoro)boron; potassium hydride (850 mg, 5.74 mmol), K₂CO₃ (397 mg, 2.87 mmol) and Pd(dppf)Cl₂ (280 mg, 0.38 mmol) at rt. The reaction mixture was degassed and purged with N₂ for 3 times, then the mixture was stirred at 120° C. for 12 h under N₂. After the reaction mixture was concentrated under reduced pressure, it was quenched with water (30 mL) and extracted with ethyl acetate (100 mL×3). The combined organic layers were washed with brine (50 mL), dried over Na₂SO₄, filtered, and concentrated. The crude product was purified by prep-HPLC to provide the desired product (68.5 mg, 14.5% yield) as a yellow solid. ¹HNMR (400 MHz, MeOH-d₄) δ 8.21-7.99 (m, 1H), 7.93-7.88 (m, 1H), 7.24-7.19 (m, 1H), 6.51 (d, J=7.2 Hz, 1H), 3.46-3.42 (m, 2H), 3.14 (s, 6H), 3.04 (t, J=7.2 Hz, 2H), 2.02-1.90 (m, 1H), 1.63-1.59 (m, 2H), 1.52-1.48 (m, 2H), 1.42 (s, 11H), 1.08-1.04 (s, 2H), 0.99-0.87 (m, 2H). MS (ESI) m/z=484.5 [M+H]⁺.

### Step 4. Synthesis of 2-((5-aminopentyl)amino)-6-cyclopropyl-N-(6-(dimethylamino)pyridazin-3-yl)nicotinamide

The title compound was synthesized following the standard procedure to provide the desired product (14 mg, 97% yield) as TFA salt. MS (ESI) m/z=384.5 [M+H]⁺.

### Example 17. 2-((5-Aminopentyl)amino)-6-cyano-N-(6-(dimethylamino)pyridazin-3-yl)nicotinamide (BL1-17)

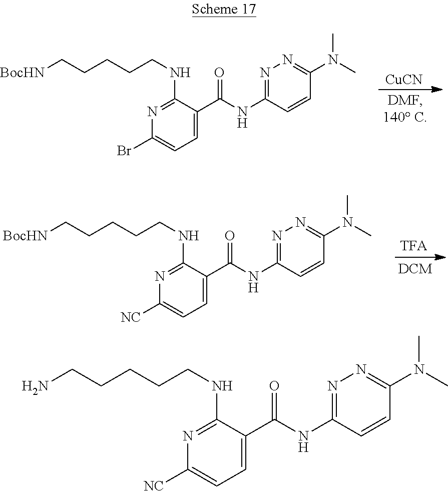

### Step 1. Synthesis of tert-butyl (5-((6-cyano-3-((6-(dimethylamino)pyridazin-3-yl)carbamoyl)pyridin-2-yl)amino)pentyl)carbamate

A mixture of tert-butyl N-[5-[[6-bromo-3-[[6-(dimethylamino)pyridazin-3-yl]carbamoyl]-2-pyridyl]amino]pentyl]carbamate (360 mg, 0.69 mmol) and CuCN (310 mg, 3.46 mmol) in DMF (5 mL) was degassed and purged with N₂ for 3 times. The reaction mixture was stirred at 140° C. for 4 h under N₂. After cooling down to rt, the reaction mixture was quenched with water (30 mL) and extracted with ethyl acetate (50 mL×3). The combined organic layers were washed with brine (50 mL), dried over Na₂SO₄, filtered, and concentrated. The residue was purified by prep-HPLC to provide the desired product (37.7 mg, 110% yield) as a yellow solid. ¹HNMR (400 MHz, DMSO-d₆) δ 8.24 (d, J=7.20 Hz, 1H), 8.14 (s, 1H), 7.89 (d, J=9.20 Hz, 1H), 7.22-7.16 (m, 1H), 6.74 (brs, 1H), 3.42-3.37 (m, 2H), 3.09 (s, 6H), 2.90 (q, J=6.00 Hz, 2H), 1.62-1.49 (m, 2H), 1.47-1.20 (m, 12H). MS (ESI) m/z=469.5 [M+H]⁺.

### Step 2. Synthesis of 2-((5-aminopentyl)amino)-6-cyano-N-(6-(dimethylamino)pyridazin-3-yl)nicotinamide

The title compound was synthesized following the standard procedure to provide the desired product (12 mg, 97% yield) as TFA salt. MS (ESI) m/z=369.5 [M+H]⁺.

### Example 18. 2-((5-aminopentyl)amino)-N-(6-isopropylpyridazin-3-yl)-6-(methylamino)nicotinamide (BL1-18)

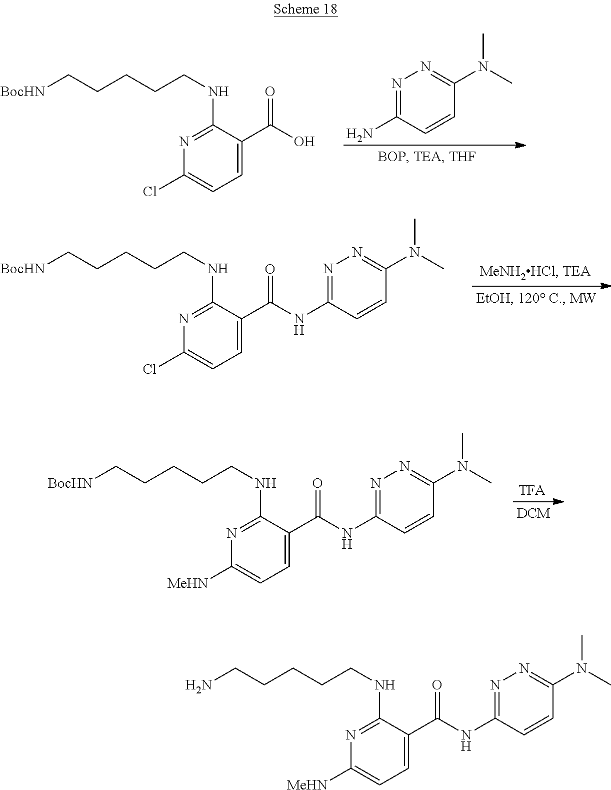

### Step 1. Synthesis of tert-butyl N-[5-[[6-chloro-3-[(6-isopropylpyridazin-3-yl)carbamoyl]-2-pyridyl]amino]pentyl]carbamate

The title compound was synthesized following the procedure of step 1 for preparing BL1-13 to provide the desired product (0.12 g, 30% yield) as yellow solid. MS (ESI) m/z=477.4 [M+H]⁺.

### Step 2. Synthesis of tert-butyl N-[5-[[3-[(6-isopropylpyridazin-3-yl)carbamoyl]-6-(methylamino)-2-pyridyl]amino]pentyl]carbamate

The title compound was synthesized following the procedure of step 2 for preparing BL1-13 to provide the desired product (50 mg, 42% yield) as a yellow solid. ¹HNMR (400 MHz, CDCl₃) δ 8.55 (s, 2H), 8.36 (d, J=9.2 Hz, 1H), 7.63 (d, J=7.6 Hz, 1H), 7.35 (d, J=9.2 Hz, 1H), 5.71 (d, J=8.8 Hz, 1H), 4.55 (s, 1H), 3.53-3.48 (m, 2H), 3.29-3.27 (m, 1H), 3.25-3.14 (m, 1H), 2.98 (d, J=5.2 Hz, 3H), 1.70-1.64 (m, 2H), 1.50-1.40 (s, 13H), 1.36 (d, J=7.2 Hz, 6H). MS (ESI) m/z=472.4 [M+H]⁺.

### Step 3. Synthesis of 2-((5-aminopentyl)amino)-N-(6-isopropylpyridazin-3-yl)-6-(methylamino)nicotinamide

The title compound was synthesized following the standard procedure to provide the desired product (13 mg, 97% yield) as a yellow solid. MS (ESI) m/z=372.5 [M+H]⁺.

### Example 19. 2-((5-aminopentyl)amino)-6-(methylamino)-N-(6-(trifluoromethyl)pyridazin-3-yl)nicotinamide (BL1-19)

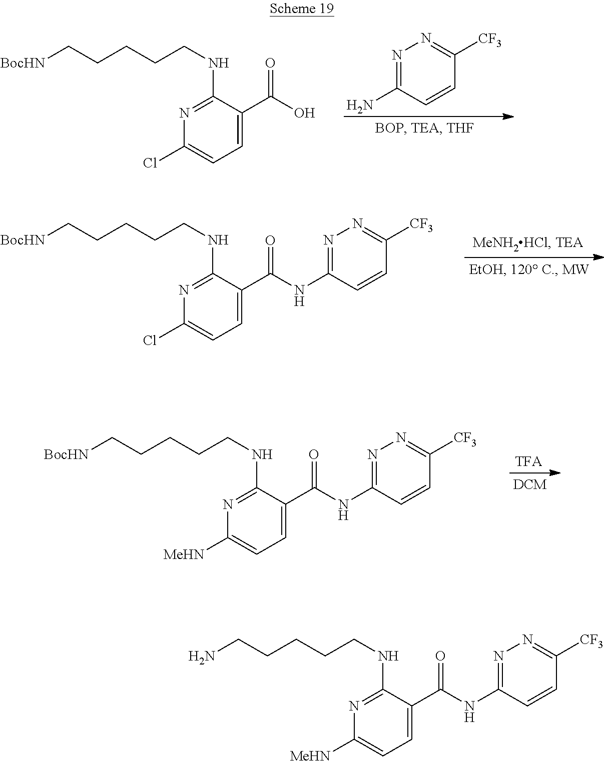 [SMILES: CC(C)(C)OC(=O)NCCCCCNc1nc(Cl)ccc1C(=O)O ; Nc1ccc(C(F)(F)F)nn1 ; CC(C)(C)OC(=O)NCCCCCNc1nc(Cl)ccc1C(=O)Nc1ccc(C(F)(F)F)nn1 ; CNc1ccc(C(=O)Nc2ccc(C(F)(F)F)nn2)c(NCCCCCNC(=O)OC(C)(C)C)n1 ; CNc1ccc(C(=O)Nc2ccc(C(F)(F)F)nn2)c(NCCCCCN)n1]

### Step 1. Synthesis of tert-butyl (5-((6-chloro-3-((6-(trifluoromethyl)pyridazin-3-yl)carbamoyl)pyridin-2-yl)amino)pentyl)carbamate

The title compound was synthesized following the procedure of step 1 for preparing BL1-13 to provide the desired product (0.28 g, 41% yield) as a yellow solid. ¹HNMR (400 MHz, CDCl₃) δ 9.55 (s, 1H), 8.67 (d, J=9.2 Hz, 1H), 8.33 (s, 1H), 7.94 (d, J=8.0 Hz, 1H), 7.86 (d, J=9.2 Hz, 1H), 6.58 (d, J=8.0 Hz, 1H), 4.54 (s, 1H), 3.44-3.66 (m, 2H), 3.16-3.13 (m, 2H), 1.66-1.71 (m, 2H), 1.53-1.59 (m, 2H), 1.44 (s, 11H). MS (ESI) m/z=503.2 [M+H]⁺.

### Step 2. Synthesis of tert-butyl (5-((6-(methylamino)-3-((6-(trifluoromethyl)pyridazin-3-yl)carbamoyl)pyridin-2-yl)amino)pentyl)carbamate

The title compound was synthesized following the procedure of step 2 for preparing BL1-13 to provide the desired product (108 mg, 47% yield) as a green solid. ¹HNMR (400 MHz, CDCl₃) δ 8.81 (s, 1H), 8.65 (d, J=9.2 Hz, 1H), 8.56 (s, 1H), 7.76 (d, J=9.6 Hz, 1H), 7.62 (d, J=8.8 Hz, 1H), 5.73 (d, J=8.8 Hz, 1H), 5.15-4.85 (m, 1H), 4.54 (s, 1H), 3.54-3.48 (m, 2H), 3.19-3.13 (m, 2H), 2.99 (d, J=4.2 Hz, 3H), 1.72-1.65 (m, 2H), 1.57-1.51 (m, 2H), 1.45 (s, 11H). MS (ESI) m/z 498.4 [M+H]⁺.

### Step 3. Synthesis of 2-((5-aminopentyl)amino)-6-(methylamino)-N-(6-(trifluoromethyl)pyridazin-3-yl)nicotinamide

The title compound was synthesized following the standard procedure to provide the desired product (15 mg, 97% yield) as a yellow solid. MS (ESI) m/z=398.6 [M+H]⁺.

### Example 20. 2-((5-Aminopentyl)amino)-N-(5-chloropyridin-2-yl)-6-(methylamino)nicotinamide (BL1-20)

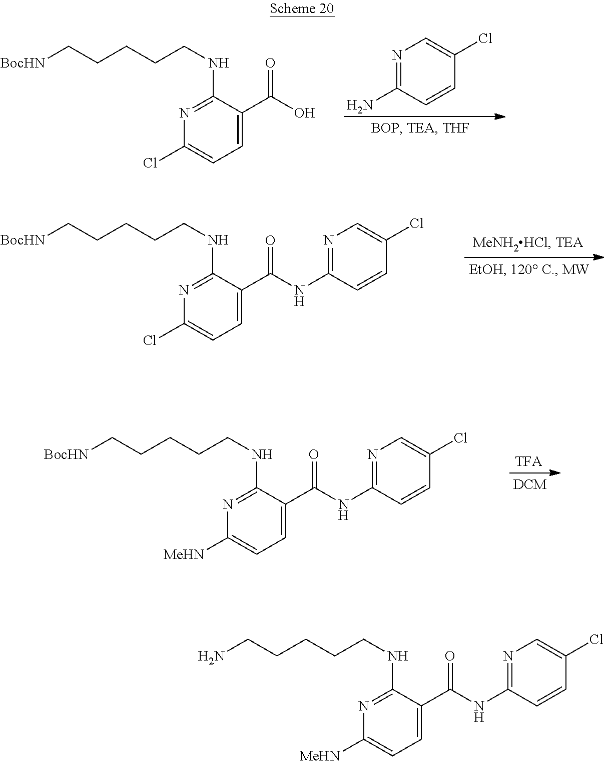 [SMILES: CC(C)(C)OC(=O)NCCCCCNc1nc(Cl)ccc1C(=O)O ; Nc1ccc(Cl)cn1 ; CC(C)(C)OC(=O)NCCCCCNc1nc(Cl)ccc1C(=O)Nc1ccc(Cl)cn1 ; CNc1ccc(C(=O)Nc2ccc(Cl)cn2)c(NCCCCCNC(=O)OC(C)(C)C)n1 ; CNc1ccc(C(=O)Nc2ccc(Cl)cn2)c(NCCCCCN)n1]

### Step 1. Synthesis of tert-butyl (5-((6-chloro-3-((5-chloropyridin-2-yl)carbamoyl)pyridin-2-yl)amino)pentyl)carbamate

To a solution of 2-[5-(tert-butoxycarbonylamino)pentylamino]-6-chloro-pyridine-3-carboxylic acid (1.00 g, 2.79 mmol) in CH₃CN (10 mL) were added 1-methylimidazole (500 mg, 6.09 mmol) and 5-chloropyridin-2-amine (400 mg, 3.11 mmol) at rt. The reaction mixture was stirred at rt for 1 h, then TCFH (1.00 g, 3.56 mmol) was added to the mixture. After the mixture was stirred at rt for additional 15 h, it was concentrated under reduced pressure. The residue was purified by prep-TLC (petroleum ether/ethyl acetate=3:1) to provide the desired product (0.39 g, 27% yield) as a yellow oil. ¹HNMR (400 MHz, CDCl₃) δ 8.47 (s, 1H), 8.38-8.31 (m, 1H), 8.29-8.20 (m, 2H), 7.77-7.68 (m, 2H), 6.54 (d, J=8.0 Hz, 1H), 4.54 (s, 1H), 3.54-3.49 (m, 2H), 3.16-3.13 (m, 2H), 1.74-1.63 (m, 4H), 1.45 (s, 11H).

### Step 2. Synthesis of tert-butyl (5-((3-((5-chloropyridin-2-yl)carbamoyl)-6-(methylamino)pyridin-2-yl)amino)pentyl)carbamate

The title compound was synthesized following the procedure of step 2 for preparing BL1-13 to provide the desired product (0.16 g, 52% yield) as a white solid. ¹HNMR (400 MHz, CDCl₃) δ 8.57 (s, 1H), 8.24-8.18 (m, 2H), 8.14 (s, 1H), 7.65 (dd, J=2.4, 8.8 Hz, 1H), 7.54 (d, J=8.4 Hz, 1H), 5.67 (d, J=8.8 Hz, 1H), 4.99-4.78 (m, 1H), 4.54 (s, 1H), 3.51-3.47 (m, 2H), 3.19-3.10 (m, 2H), 2.97 (d, J=5.2 Hz, 3H), 1.71-1.63 (m, 2H), 1.56-1.51 (m, 2H), 1.45 (s, 11H), MS (ESI) m/z=463.2 [M+H]⁺.

### Step 3. Synthesis of 2-((5-aminopentyl)amino)-N-(5-chloropyridin-2-yl)-6-(methylamino)nicotinamide

The title compound was synthesized following the standard procedure to provide the desired product (15 mg, 97% yield) as a yellow solid. MS (ESI) m/z=363.4 [M+H]⁺.

### Example 21. 2-((6-Aminohexan-2-yl)amino)-N-(6-(dimethylamino)pyridazin-3-yl)-6-(methylamino)nicotinamide (BL1-21)

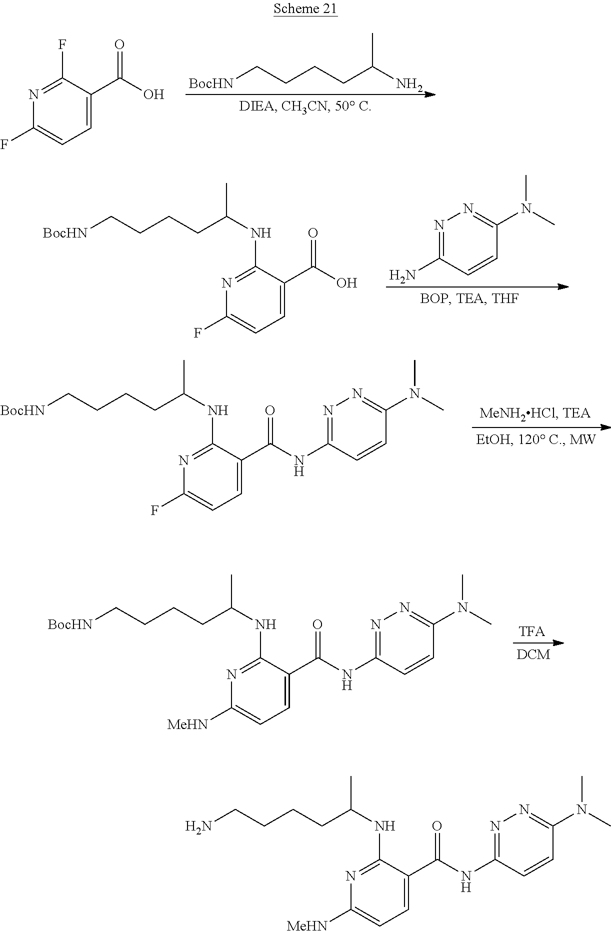

### Step 1. Synthesis of 2-((6-((tert-butoxycarbonyl)amino)hexan-2-yl)amino)-6-fluoronicotinic acid

To a solution of tert-butyl N-(5-aminohexyl) carbamate (1.20 g, 5.55 mmol) and 2,6-difluoropyridine-3-carboxylic acid (600 mg, 3.77 mmol) in CH₃CN (6 mL) was added DIEA (1.78 g, 13.7 mmol) at rt. After the reaction mixture was stirred at 50° C. for 2 h, it was concentrated under vacuum. The residue was purified by prep-TLC (DCM/MeOH=10:1) to provide the desired product (0.40 g, crude) as a yellow oil. ¹HNMR (400 MHz, CDCl₃) δ 8.25 (t, J=8.4 Hz, 1H), 8.06-7.92 (m, 1H), 6.12-6.02 (m, 1H), 4.55 (s, 1H), 4.21 (d, J=5.6 Hz, 1H), 3.16-3.08 (m, 2H), 1.63-1.52 (m, 4H), 1.44 (brs, 11H), 1.23 (d, J=6.0 Hz, 3H).

### Step 2. Synthesis of tert-butyl (5-((3-((6-(dimethylamino)pyridazin-3-yl)carbamoyl)-6-fluoropyridin-2-yl)amino)hexyl)carbamate

The title compound was synthesized following the procedure of step 1 for preparing BL1-13 to provide the desired product (110 mg, 31% yield) as a yellow solid. ¹HNMR (400 MHz, CDCl₃) δ 8.42-8.40 (m, 1H), 8.18 (d, J=9.6 Hz, 1H), 8.07 (t, J=8.4 Hz, 1H), 6.95 (d, J=9.6 Hz, 1H), 6.10 (dd, J=2.8, 8.0 Hz, 1H), 4.52 (s, 1H), 4.27-4.17 (m, 1H), 3.17 (s, 6H), 3.14-3.10 (m, 2H), 1.55-1.48 (m, 4H), 1.43 (s, 11H), 1.23 (d, J=6.4 Hz, 3H).

### Step 3. Synthesis of tert-butyl (5-((3-((6-(dimethylamino)pyridazin-3-yl)carbamoyl)-6-(methylamino)pyridin-2-yl)amino)hexyl)carbamate

The title compound was synthesized following the procedure of step 2 for preparing BL1-13 to provide the desired product (100 mg, 85% yield) as a yellow solid. ¹ HNMR (400 MHz, CDCl₃) δ 8.42-8.34 (m, 2H), 8.17 (d, J=9.6 Hz, 1H), 7.61 (d, J=8.8 Hz, 1H), 6.89 (d, J=10.0 Hz, 1H), 5.67 (d, J=8.8 Hz, 1H), 5.00-4.83 (m, 1H), 4.55 (s, 1H), 4.32-4.23 (m, 1H), 3.15 (s, 6H), 3.13-3.07 (m, 2H), 2.95 (d, J=5.2 Hz, 3H), 1.57-1.49 (m, 4H), 1.44 (s, 11H), 1.22 (d, J=6.4 Hz, 3H), MS (ESI) m/z=487.5 [M+H]⁺.

### Step 4. Synthesis of 2-((6-aminohexan-2-yl)amino)-N-(6-(dimethylamino)pyridazin-3-yl)-6-(methylamino)nicotinamide

The title compound was synthesized following the standard procedure to provide the desired product (11 mg, 97% yield) as a yellow solid. MS (ESI) m/z=387.6 [M+H]⁺.

### Example 22. 2-((5-Aminohexyl)amino)-N-(6-(dimethylamino)pyridazin-3-yl)-6-(methylamino)nicotinamide (BL1-22)

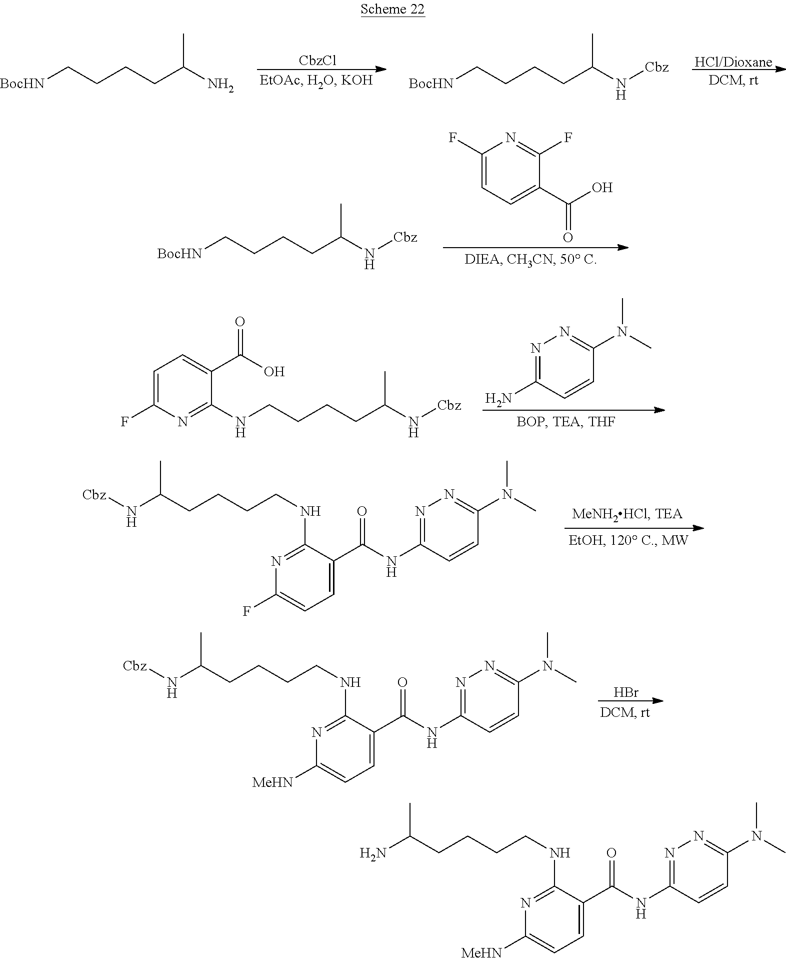

### Step 1. Synthesis of tert-butyl N-[5-(benzyloxycarbonylamino)hexyl]carbamate

To a solution of tert-butyl N-(5-aminohexyl)carbamate (1.00 g, 4.62 mmol) and CbzCl (1.08 g, 6.33 mmol) in EtOAc (10 mL) was added a solution of KOH (520 mg, 9.27 mmol) in H₂O (10 mL) at rt. After the mixture was stirred at rt for 1 h, it was diluted with H₂O (20 mL) and extracted with EtOAc (30 mL×2). The combined organic layers were washed with brine (30 mL), dried over Na₂SO₄, filtered, and concentrated. The residue was purified by silica gel column chromatography (petroleum ether/ethyl acetate=1:0 to 3:1) to provide the desired product (1.10 g, 68% yield) as a yellow oil. ¹HNMR (400 MHz, CDCl₃) δ 7.40-7.28 (m, 5H), 5.15-5.04 (m, 2H), 4.60-4.57 (m, 2H), 3.78-3.64 (m, 1H), 3.13-3.09 (m, 2H), 1.44 (s, 11H), 1.38-1.33 (m, 2H), 1.14 (d, J=6.8 Hz, 3H).

### Step 2. Synthesis of benzyl N-(5-amino-1-methyl-pentyl)carbamate

To a solution of tert-butyl N-[5-(benzyloxycarbonylamino)hexyl]carbamate (1.10 g, 3.14 mmol) in DCM (10 mL) was added HCl solution (4 M in dioxane, 6 mL) at rt. After the mixture was stirred at rt for 12 h, it was concentrated under vacuum to provide the desired product (0.80 g, 88% yield) as a white solid. ¹HNMR (400 MHz, DMSO-d₆) δ 7.40-7.28 (m, 5H), 7.13 (d, J=8.4 Hz, 1H), 5.00 (s, 2H), 3.51-3.44 (m, 1H), 2.74 (t, J=7.6 Hz, 2H), 1.61-1.22 (m, 8H), 1.04 (d, J=6.8 Hz, 3H).

### Step 3. Synthesis of 2-[5-(benzyloxycarbonylamino) hexylamino]-6-fluoro-pyridine-3-carboxylic acid

The title compound was synthesized following the procedure of step 1 for preparing BL1-13 to provide the desired product (550 mg, crude) as a yellow oil. ¹HNMR (400 MHz, CDCl₃) J=8.88 (s, 1H), 8.25-8.22 (s, 1H), 7.39-7.28 (m, 51H), 5.98 (d, J=5.2 Hz, 1H), 5.13-4.99 (m, 2H), 4.67 (d, J=7.6 Hz, 1H), 3.72-3.65 (m, 3H), 1.48-1.45 (m, 4H), 1.44-1.40 (m, 11H), 1.13 (d, J=6.4 Hz, 3H).

### Step 4. Synthesis of benzyl N-[5-[[3-[[6-(dimethylamino)pyridazin-3-yl]carbamoyl]-6-fluoro-2-pyridyl]amino]-1-methyl-pentyl]carbamate

The title compound was synthesized following the procedure of step 1 for preparing BL1-13 to provide the desired product (0.23 g, 35% yield) as a yellow oil. ¹HNMR (400 MHz, CDCl₃) δ 8.54-8.51 (m, 1H), 8.15-8.06 (m, 2H), 7.37-7.29 (m, 5H), 6.91 (d, J=10.0 Hz, 1H), 6.10 (dd, J=2.8, 8.4 Hz, 1H), 5.13-5.03 (m, 2H), 4.57 (d, J=8.4 Hz, 1H), 3.79-3.68 (m, 1H), 3.47 (q, J=6.4 Hz, 2H), 3.16 (s, 6H), 1.50-1.45 (m, 6H), 1.15 (d, J=6.4 Hz, 3H).

### Step 5. Synthesis of benzyl N-[5-[[3-[[6-(dimethylamino)pyridazin-3-yl]carbamoyl]-6-(methylamino)-2-pyridyl]amino]-1-methyl-pentyl]carbamate

The title compound was synthesized following the procedure of step 2 for preparing BL1-13 to provide the desired product (0.22 g, 93% yield) as a yellow solid. ¹HNMR (400 MHz, CDCl₃) δ 8.54-8.53 (m, 1H), 8.35 (s, 1H), 8.17 (d, J=9.6 Hz, 1H), 7.60 (d, J=8.4 Hz, 1H), 7.36-7.30 (m, 5H), 6.86 (d, J=10.0 Hz, 1H), 5.67 (d, J=8.4 Hz, 1H), 5.12-5.05 (m, 2H), 4.83 (s, 1H), 4.60 (brd, J=8.4 Hz, 1H), 3.76-3.72 (m, 1H), 3.49-3.43 (m, 2H), 3.14 (s, 6H), 2.95 (d, J=4.8 Hz, 3H), 1.56-1.32 (m, 6H), 1.15 (d, J=6.4 Hz, 3H)

### Step 6. Synthesis of 2-(5-aminohexylamino)-N-[6-(dimethylamino)pyridazin-3-yl]-6-(methylamino)pyridine-3-carboxamide

To a solution of benzyl N-[5-[[3-[[6-(dimethylamino)pyridazin-3-yl]carbamoyl]-6-(methylamino)-2-pyridyl]amino]-1-methyl-pentyl]carbamate (200 mg, 0.38 mmol) in DCM (2 mL) was added HBr (372 mg, 1.52 mmol, 0.25 mL, 33 wt %) at rt. After the mixture was stirred at rt for 2 h, it was poured into H₂O (5 mL) and the aqueous layer was extracted with ethyl acetate (5 mL×2). The aqueous layer was adjusted pH to 10 with 2N NaOH (2 mL), then extracted with DCM/MeOH (v/v=10:1, 20 mL×3). The organic layer was washed with brine (30 mL), dried over Na₂SO₄, filtered, and concentrated. The residue was purified by prep-TLC (DCM/MeOH=10:1) to provide the desired product (0.06 g, 37% yield) as a yellow solid. ¹′HNMR (400 MHz, DMSO-d₆) δ 10.09-10.02 (m, 1H), 8.74 (s, 1H), 7.96 (brd, J=8.8 Hz, 1H), 7.80 (d, J=10.0 Hz, 1H), 7.14 (d, J=10.0 Hz, 1H), 6.94-6.87 (m, 1H), 5.69 (d, J=8.8 Hz, 1H), 3.42-3.41 (m, 4H), 3.08 (s, 6H), 2.82 (d, J=4.4 Hz, 3H), 1.57-1.55 (m, 2H), 1.36 (s, 4H), 1.11 (d, J=6.4 Hz, 3H), MS (ESI) m/z=387.2 [M+H]⁺.

### Example 23. 2-((4-Azidobutyl)amino)-N-(6-(dimethylamino)pyridazin-3-yl)-6-(methylamino)nicotinamide (BL1-23)

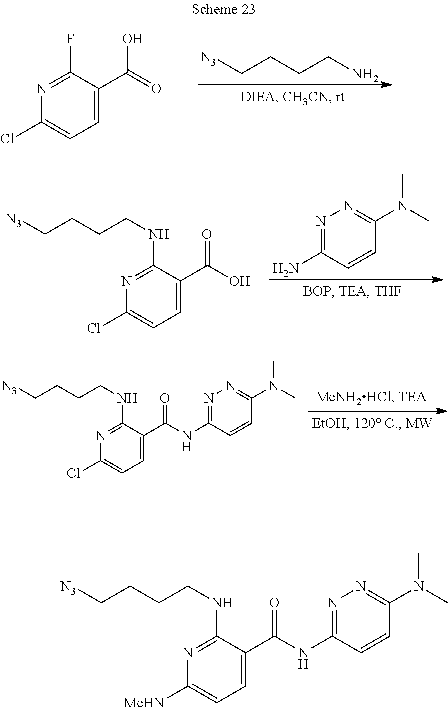

### Step 1. Synthesis of 2-(4-azidobutylamino)-6-chloro-pyridine-3-carboxylic acid

To a mixture of 6-chloro-2-fluoro-pyridine-3-carboxylic acid (300 mg, 1.71 mmol) and 4-azidobutan-1-amine hydrochloride (420 mg, 2.79 mmol) in CH₃CN (5 mL) was added DIEA (1.48 g, 11.5 mmol) at rt. The reaction mixture was stirred at rt for 12 h, before it was concentrated under reduced pressure. The residue was purified by prep-TLC (dichloromethane/methanol=10:1) to provide the desired product (0.24 g, 32% yield) as a yellow oil. MS (ESI) m/z=270.2 [M+H]⁺.

### Step 2. Synthesis of 2-(4-azidobutylamino)-6-chloro-N-[6-(dimethylamino)pyridazin-3-yl]pyridine-3-carboxamide

The title compound was synthesized following the procedure of step 1 for preparing BL1-13 to provide the desired product (0.15 g, 69% yield) as a yellow oil. 1HNMR (400 MHz, CDCl₃) δ 8.44 (s, 1H), 8.10 (d, J=8.0 Hz, 2H), 6.94 (d, J=10.0 Hz, 1H), 6.52 (d, J=8.0 Hz, 1H), 3.58-3.52 (m, 1H), 3.35 (t, J=6.4 Hz, 2H), 3.16 (s, 6H), 1.76-1.69 (m, 4H). MS (ESI) m/z=390.3 [M+H]⁺.

### Step 3. Synthesis of 2-(4-azidobutylamino)-N-[6-(dimethylamino)pyridazin-3-yl]-6-(methylamino)pyridine-3-carboxamide

The title compound was synthesized following the procedure of step 2 for preparing BL1-13 to provide the desired product (80.8 mg, 46% yield) as a yellow solid. ¹HNMR (400 MHz, CDCl₃) δ 8.58 (s, 1H), 8.34 (s, 1H), 8.18 (d, J=9.6 Hz, 1H), 7.61 (d, J=8.8 Hz, 1H), 6.89 (d, J=9.6 Hz, 1H), 5.70 (d, J=8.8 Hz, 1H), 4.86-4.66 (m, 1H), 3.60-3.50 (m, 1H), 3.40-3.25 (t, J=6.0 Hz, 2H), 3.20-3.07 (m, 6H), 2.97 (d, J=4.8 Hz, 3H), 1.76-1.70 (m, 4H). MS (ESI) m/z=385.3 [M+H]⁺.

### Example 24. 2-((1-(2-Aminoethyl)-1H-pyrazol-4-yl)amino)-N-(6-(dimethylamino)pyridazin-3-yl)-6-(methylamino)nicotinamide (BL1-24)

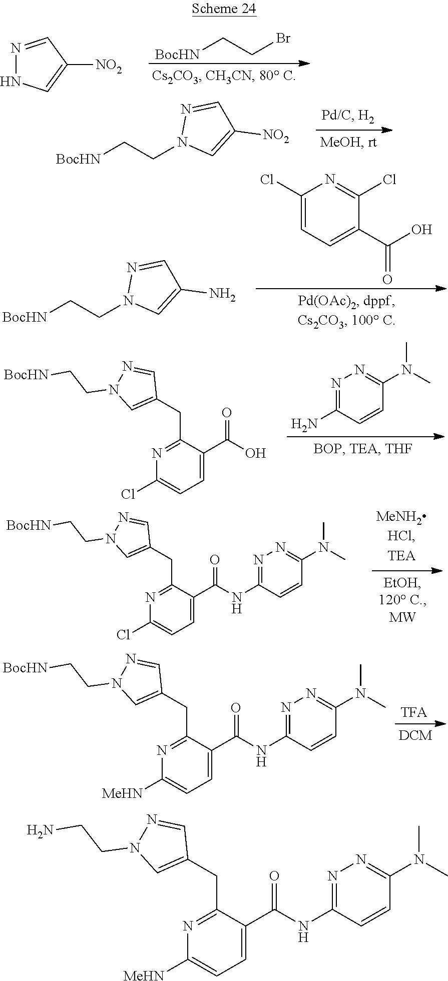

### Step 1. Synthesis of tert-butyl N-[2-(4-nitropyrazol-1-yl)ethyl]carbamate

To a mixture of 4-nitro-1H-pyrazole (1.00 g, 8.84 mmol) and Cs₂CO₃ (3.46 g, 10.6 mmol) in CH₃CN (10 mL) was added tert-butyl N-(2-bromoethyl)carbamate (2. 18 g, 9.73 mmol) at rt. The reaction mixture was stirred at 80 for 1.5 h. After cooling down to rt, the reaction mixture was filtered and the filtrate was concentrated to provide the desired product (2.20 g, crude) as a brown solid. ¹HNMR (400 MHz, CDCl₃) δ 8.14 (s, 1H), 8.10 (s, 1H), 4.80 (s, 1H), 4.33-4.26 (n, 2H), 3.62-3.58 (m, 2H), 1.44 (s, 9H). MS (ESI) m/z=157.3 [M+H]⁺.

### Step 2. Synthesis of tert-butyl N-[2-(4-aminopyrazol-1-yl)ethyl]carbamate

To a mixture of tert-butyl N-[2-(4-nitropyrazol-1-yl)ethyl]carbamate (2.20 g, 8.59 mmol) in MeOH (30 mL) was added 10% Pd/C (0.30 g, 8.59 mmol) at rt. The reaction mixture was stirred at rt for 18 h under H₂. The mixture was filtered and the filtrate was concentrated in vacuum to provide the desired product (1.90 g, 97% yield) as brown solid. ¹HNMR (400 MHz, DMSO-d₆) δ 6.974 (s, 1H), 6.90 (s, 1H), 6.86-6.80 (m, 1H), 3.93 (t, J=6.8 Hz, 2H), 3.24-3.18 (s, 2H), 1.37 (s, 9H).

### Step 3. Synthesis of 2-[[1-[2-(tert-butoxycarbonylamino)ethyl]pyrazol-4-yl]amino]-6-chloro-pyridine-3-carboxylic acid

To a mixture of 2,6-dichloropyridine-3-carboxylic acid (0.30 g, 1.56 mmol) and tert-butyl N-[2-(4-aminopyrazol-1-yl)ethyl]carbamate (700 mg, 3.09 mmol) in dioxane (5 mL) were added (5-diphenylphosphanyl-9,9-dimethyl-xanthen-4-yl)-diphenyl-phosphane (150 mg, 0.26 mmol), Pd(OAc)₂ (30 mg, 0.13 mmol) and Cs₂CO₃ (3.00 g, 9.21 mmol) at rt under N₂. The reaction mixture was stirred at 100° C. for 2 h. After cooling down to rt, the reaction mixture was poured into water (20 mL) and extracted with ethyl acetate (20 mL×3). The combined organic phase was washed with brine (30 mL), dried over anhydrous Na₂SO₄, filtered, and concentrated in vacuum. The residue was purified by prep-HPLC to provide the desired product (0.14 g, 24% yield) as a brown solid. ¹HNMR (400 MHz, CDCl₃) δ 9.84 (s, 1H), 8.30 (s, 1H), 7.84 (d, J=8.0 Hz, 1H), 7.52 (s, 1H), 7.10-7.00 (m, 1H), 6.36 (d, J=8.0 Hz, 1H), 4.29-4.16 (m, 2H), 3.59-3.57 (m, 2H), 1.61 (s, 9H). MS (ESI) m/z=382.1 [M+H]⁺.

### Step 4. Synthesis of tert-butyl N-[2-[4-[[6-chloro-3-[[6-(dimethylamino)pyridazin-3-yl]carbamoyl]-2-pyridyl]amino]pyrazol-1-yl]ethyl]carbamate

The title compound was synthesized following the procedure of step 2 for preparing BL1-13 to provide the desired product (0.17 g, 75% yield) as a brown solid. MS (ESI) m/z=502.4 [M+H]⁺.

### Step 5. Synthesis of tert-butyl N-[2-[4-[[3-[[6-(dimethylamino)pyridazin-3-yl]carbamoyl]-6-(methylamino)-2-pyridyl]amino]pyrazol-1-yl]ethyl]carbamate

The title compound was synthesized following the procedure of step 2 for preparing BL1-13 to provide the desired product (108 mg, 47% yield) as a white solid. ¹HNMR (400 MHz, CDCl₃) δ 10.68 (s, 11H), 8.75-8.43 (m, 1H), 8.21 (d, J=10.0 Hz, 1H), 7.95 (s, 1H), 7.82-7.64 (m, 2H), 6.93 (d, J=9.6 Hz, 1H), 5.84 (d, J=8.8 Hz, 1H), 4.99-4.30 (m, 2H), 4.20 (t, J=5.2 Hz, 214), 3.61-3.59 (m, 214), 3.17 (s, 6H), 3.04 (d, J=4.8 Hz, 3H), 1.44 (s, 914). MS (ESI) m/z=497.4 [M+H]⁺.

### Step 6. Synthesis of 2-((1-(2-aminoethyl)-1H-pyrazol-4-yl)amino)-N-(6-(dimethylamino)pyridazin-3-yl)-6-(methylamino)nicotinamide

The title compound was synthesized following the standard procedure to provide the desired product (11 mg, 97% yield) as a yellow solid. MS (ESI) m/z=397.5 [M+H]⁺.

### Example 25. 2-((5-Aminopentyl)(methyl)amino)-N-(6-(dimethylamino)pyridazin-3-yl)-6-(methylamino)nicotinamide (BL1-25)

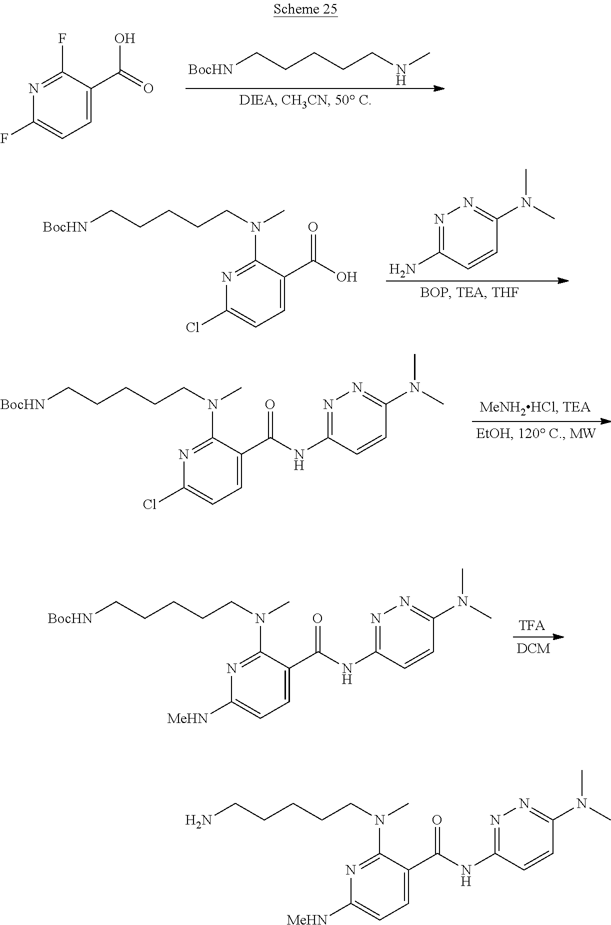

### Step 1. Synthesis of 2-[5-(tert-butoxycarbonylamino)pentyl-methyl-amino]-6-chloro-pyridine-3-carboxylic acid

The title compound was synthesized following the procedure of step 1 for preparing BL1-23 to provide the desired product (0.31 g, 69% yield) as a light-yellow oil. ¹HNMR (400 MHz, CDCl₃) δ 8.36 (d, J=7.6 Hz, 1H), 7.25-7.16 (m, 1H), 4.55 (s, 1H), 3.27-3.16 (m, 2H), 3.14-3.04 (m, 2H), 2.85 (s, 3H), 1.56-1.51 (m, 2H), 1.50-1.38 (m, 11H), 1.35-1.32 (m, 2H). MS (ESI) m/z=372.1 [M+H]⁺.

### Step 2. Synthesis of tert-butyl N-[5-[[6-chloro-3-[[6-(dimethylamino)pyridazin-3-yl]carbamoyl]-2-pyridyl]-methyl-amino]pentyl]carbamate

The title compound was synthesized following the procedure of step 1 for preparing BL1-13 to provide the desired product (0.30 g, 73% yield) as a yellow oil. ¹HNMR (400 MHz, CDCl₃) δ 10.93 (s, 1H), 8.36 (d, J=10.0 Hz, 1H), 8.24 (d, J=8.0 Hz, 1H), 7.06 (d, J=8.0 Hz, 1H), 6.92 (d, J=10.0 Hz, 1H), 4.84 (s, 1H), 3.36-3.28 (m, 2H), 3.18 (s, 6H), 3.11-3.08 (m, 2H), 2.92 (s, 3H), 1.69-1.63 (m, 2H), 1.52-1.46 (m, 2H), 1.41 (s, 9H), 1.35-1.30 (m, 2H). MS (ESI) m/z=492.4 [M+H]⁺.

### Step 3. Synthesis of tert-butyl N-[5-[[3-[[6-(dimethylamino)pyridazin-3-yl]carbamoyl]-6-(methylamino)-2-pyridyl]-methyl-amino]pentyl]carbamate

The title compound was synthesized following the procedure of step 2 for preparing BL1-13 to provide the desired product (55.7 mg, 18% yield) as a brown gum. ¹HNMR (400 MHz, CDCl₃) δ 12.13 (s, 1H), 8.48-8.37 (m, 1H), 8.27-8.15 (m, 1H), 6.89 (d, J=10.0 Hz, 1H), 6.19 (d, J=8.4 Hz, 1H), 4.96 (s, 2H), 3.20-3.13 (m, 8H), 3.04-3.01 (m, 2H), 2.97-2.94 (m, 3H), 2.79 (s, 3H), 1.63-1.50 (m, 2H), 1.40 (s, 9H), 1.32-1.24 (m, 2H). MS (ESI) m/z=487.5 [M+H]⁺.

### Step 4. Synthesis of 2-((5-aminopentyl)(methyl)amino)-N-(6-(dimethylamino)pyridazin-3-yl)-6-(methylamino)nicotinamide

The title compound was synthesized following the standard procedure to provide the desired product (13 mg, 90% yield) as a yellow solid. MS (ESI) m/z=387.5 [M+H]⁺.

### Example 26. N-(6-(Dimethylamino)pyridazin-3-yl)-2-((2-hydroxyethyl)amino)-6-(methylamino)nicotinamide (BL1-26)

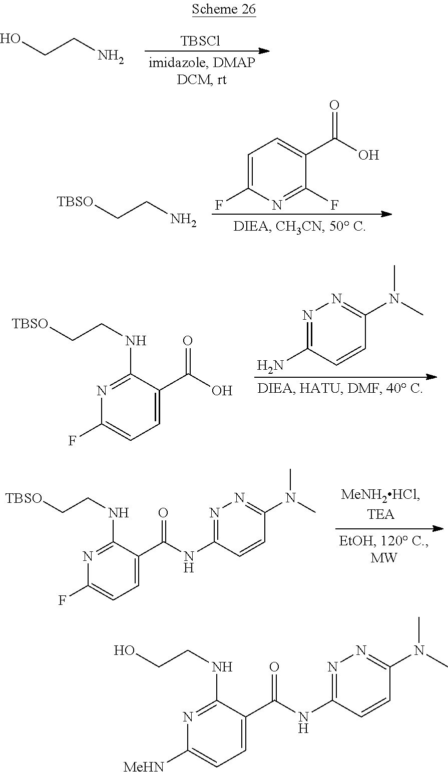

### Step 1. Synthesis of 2-((tert-butyldimethylsilyl)oxy)ethanamine

To a solution of 2-aminoethanol (2.00 g, 32.0 mmol) in DCM (10 mL) were added imidazole (6.70 g, 98.0 mmol), DMAP (400 mg, 3.27 mmol) and TBSCl (5.50 g, 36.5 mmol) at 0 [symbol] After the reaction mixture was stirred at rt for 5 h, it was diluted with H₂O (20 mL) and extracted with ethyl acetate (30 mL×3). The combined organic layers were washed with brine (30 mL), dried over anhydrous Na₂SO₄, filtered, and concentrated to provide the desired product (5.74 g, 100% yield) as a colorless oil. ¹HNMR (400 MHz, CDCl₃) δ 7.75 (s, 1H), 3.76 (t, J=5.2 Hz, 2H), 2.95 (m, 2H), 0.83 (s, 9H), 0.04 (s, 6H).

### Step 2. Synthesis of 2-((2-((tert-butyldimethylsilyl)oxy)ethyl)amino)-6-fluoronicotinic acid

To a solution of 2-[tert-butyl(dimethyl)silyl]oxyethanamine (5.74 g, 32.7 mmol) and 2,6-difluoropyridine-3-carboxylic acid (5.21 g, 32.0 mmol) in CH₃CN (50 mL) was added DIEA (13.0 g, 100 mmol) at rt. The reaction mixture was stirred at 50 [symbol] for 1 h. Then the reaction mixture was diluted with citric acid (20 mL) and extracted with ethyl acetate (30 mL×3). The combined organic layers were washed with brine (30 mL), dried over anhydrous Na₂SO₄, filtered, and concentrated under reduced pressure. The residue was purified by silica gel column chromatography (DCM/EtOAc=1:0) to provide the desired product (2.09 g, 20% yield) as a white solid. ¹HNMR (400 MHz, CDCl₃) δ 8.35 (s, 1H), 6.12 (dd, J=2.8 Hz, 8.4 Hz, 1H), 3.82-3.81 (m, 2H), 3.80-3.78 (m, 2H), 0.83 (s, 9H), 0.10 (s, 6H). MS (ESI) m/z=315.1 [M+H]⁺.

### Step 3. Synthesis of 2-((2-((tert-butyldimethylsilyl)oxy)ethyl)amino)-N-(6-(dimethylamino)pyridazin-3-yl)-6-fluoronicotinamide

To a solution of 2-[2-[tert-butyl(dimethyl)silyl]oxyethylamino]-6-fluoro-pyridine-3-carboxylic acid (100 mg, 0.32 mmol), N³,N³-dimethylpyridazine-3,6-diamine (50 mg, 0.36 mmol) in DMF (1 mL) were added DIEA (126 mg, 0.98 mmol) and HATU (150 mg, 0.39 mmol) at rt. The reaction mixture was stirred at 40° C. for 8 h. The reaction mixture was diluted with H₂O (20 mL) and extracted with ethyl acetate (30 mL×3). The combined organic layers were washed with brine (30 mL), dried over anhydrous Na₂SO₄, filtered, and concentrated. The residue was purified by silica gel column chromatography (petroleum ether/ethyl acetate=5:1 to 1:1) to provide the desired product (710 mg, 26% yield) as a brown oil. MS (ESI) m/z=435.2 [M+H]⁺.

### Step 4. Synthesis of N-(6-(dimethylamino)pyridazin-3-yl)-2-((2-hydroxyethyl)amino)-6-(methylamino)nicotinamide

To a solution of 2-[2-[tert-butyl(dimethyl)silyl]oxyethylamino]-N-[6-(dimethylamino)pyridazin-3-yl]-6-fluoro-pyridine-3-carboxamide (710 mg, 1.63 mmol) in EtOH (5 mL) were added methanamine hydrochloride (1.10 g, 16.3 mmol) and DIEA (2.11 g, 16.3 mmol, 2.85 mL) at rt. The reaction mixture was heated at 120 [symbol] for 4 h under microwave irradiation. After cooling down to rt, the reaction mixture was diluted with H₂O (20 mL) and extracted with ethyl acetate (30 mL×3). The combined organic layers were washed with brine (30 mL), dried over anhydrous Na₂SO₄, filtered, and concentrated under reduced pressure. The residue was purified by prep- HPLC (0.1% NH₃—H₂O) to provide the desired product (270 mg, 47% yield) as a brown solid. ¹HNMR (400 MHz, MeOH-d4) δ 8.01 (d, J=9.6 Hz, 11H), 7.79 (d, J=8.8 Hz, 1H), 7.18 (d, J=10.0 Hz, 1H), 5.78 (d, J=9.2 Hz, 1H), 3.75-3.73 (m, 2H), 3.66-3.62 (m, 2H), 3.14 (s, 6H), 2.91 (s, 3H). MS (ESI) m/z=332.4 [M+H]⁺.

### Example 27. N-(6-(Dimethylamino)pyridazin-3-yl)-2-((3-hydroxypropyl)amino)-6-(methylamino)nicotinamide (BL1-27)

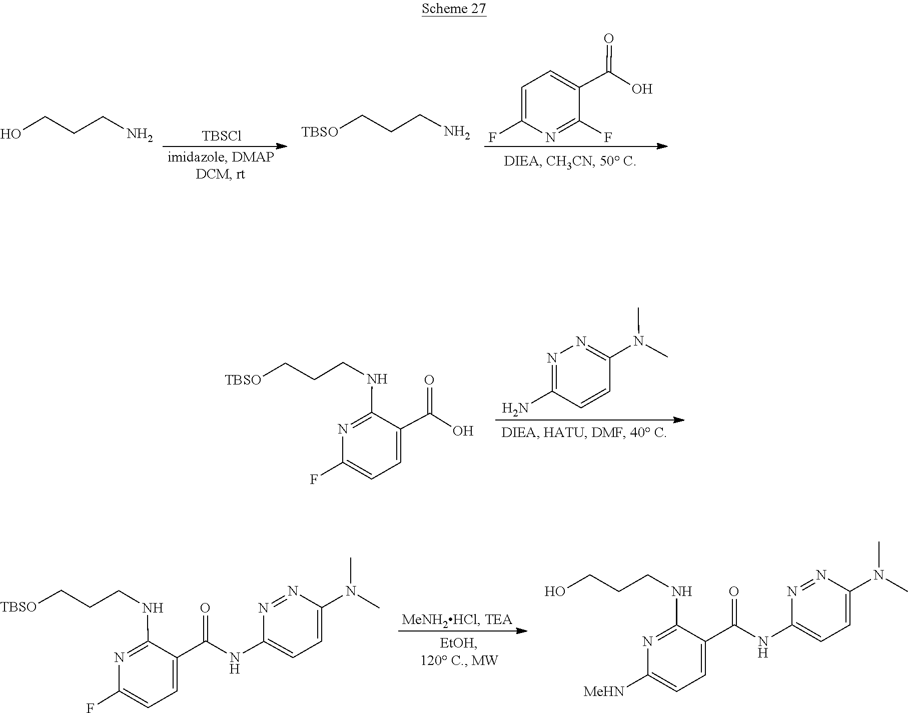

BL1-27 was synthesized following the procedures for preparing BL1-26 (189 mg, 2% yield over 4 steps) as a green solid. ¹HNMR (400 MHz, MeOH-d₄) δ 8.00 (d, J=10.0 Hz, 11H), 7.78 (d, J=8.8 Hz, 11H), 7.17 (d, J=10.0 Hz, 1H), 5.75 (d, J=8.8 Hz, 1H), 3.67-3.58 (m, 4H), 3.13 (s, 6H), 2.91 (s, 3H), 1.87-1.81 (m, 2H). MS (ESI) m/z=346.3 [M+H]⁺.

### Example 28. N-(6-(dimethylamino)pyridazin-3-yl)-2-((4-hydroxybutyl)amino)-6-(methylamino)nicotinamide (BL1-28)

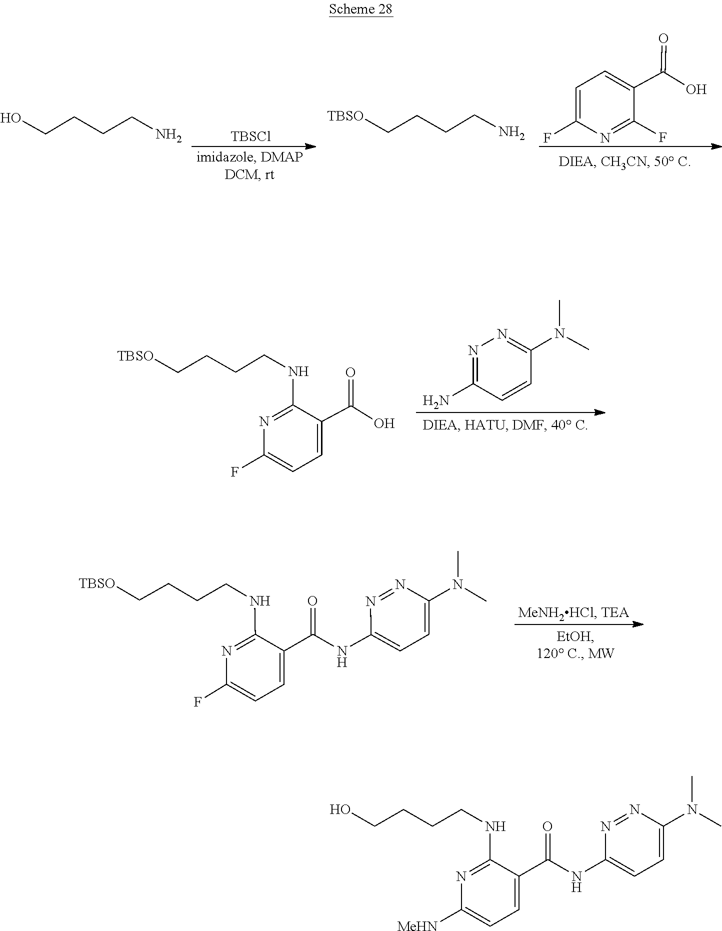

BL1-28 was synthesized following the procedures for preparing BL1-26 (107 mg, 1% yield over 4 steps) as a yellow solid. ¹ HNMR (400 MHz, MeOH-d₄) b 7.99 (d, J=10.0, 1H), 7.77 (d, J=8.8, 2H), 7.18 (d, J=9.6, 1H), 5.74 (d, J=8.8, 1H), 3.60 (t, J=6.8, 2H), 3.53 (t, J=6.8, 2H), 3.13 (s, 6H), 2.91 (s, 3H), 1.69-1.62 (m, 4H). MS (ESI) m/z=360.1 [M+H]⁺.

### Example 29. N-(6-(dimethylamino)pyridazin-3-yl)-2-((5-hydroxypentyl)amino)-6-(methylamino)nicotinamide (BL1-29)

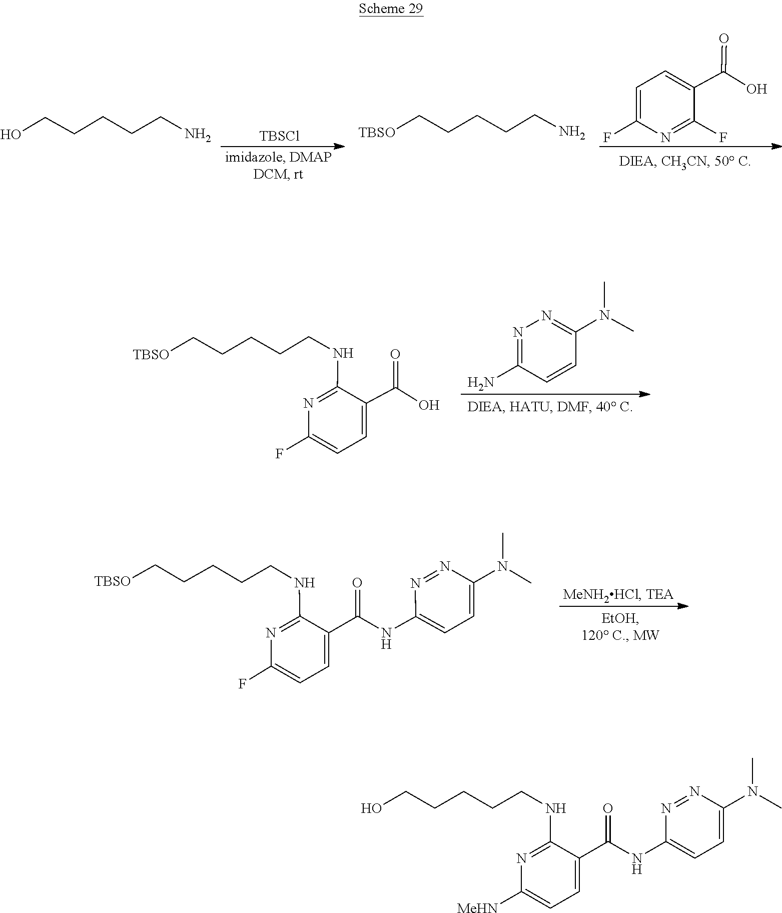

BL1-29 was synthesized following the procedures for preparing BL1-26 (150 mg, 2% yield over 4 steps) as a green solid. ¹HNMR (400 MHz, MeOH-d₄) δ 7.99 (d, J=9.2 Hz, 1H), 7.77 (d, J=8.8 Hz, 1H), 7.19 (d, J=10.0 Hz, 1H), 5.74 (d, J=8.8 Hz, 1H), 3.58-3.54 (m, 2H), 3.52-3.49 (m, 2H), 3.13 (s, 6H), 2.91 (s, 3H), 1.68-1.57 (m, 4H), 1.48-1.47 (m, 2H). MS (ESI) m/z=374.1 [M+H]⁺.

### Example 30. N-(6-(dimethylamino)pyridazin-3-yl)-2-((6-hydroxyhexyl)amino)-6-(methylamino)nicotinamide (BL1-30)

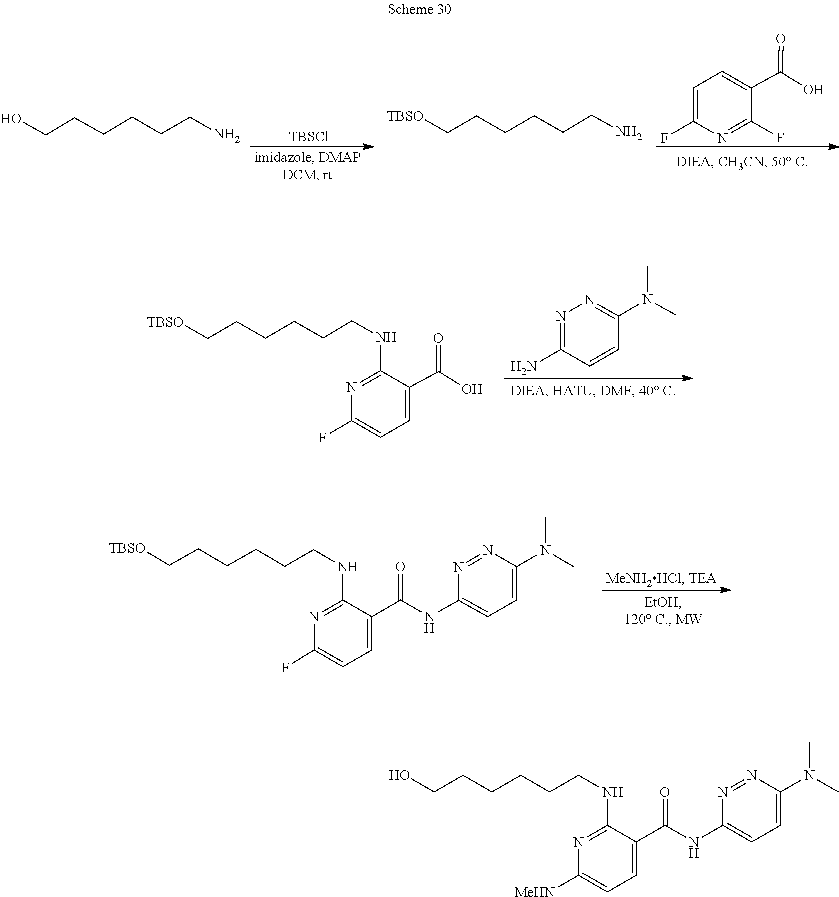

BL1-30 was synthesized following the procedures for preparing BL1-26 (211 mg, 3% yield over 4 steps) as a white solid. ¹HNMR (400 MHz, CDCl₃) δ 8.00 (d, J=10.0 Hz, 1H), 7.77 (d, J=8.8 Hz, 1H), 7.21 (d, J=9.6 Hz, 1H), 5.74 (d, J=8.8 Hz, 1H), 3.57-3.50 (m, 5H), 3.14 (s, 6H), 2.92 (s, 3H), 1.68-1.63 (m, 2H), 1.58-1.54 (m, 2H), 1.44-1.42 (m, 5H). MS (ESI) m/z=388.5 [M+H]⁺.

### Example 31. N-(6-(dimethylamino)pyridazin-3-yl)-2-((7-hydroxyheptyl)amino)-6-(methylamino)nicotinamide (BL1-31)

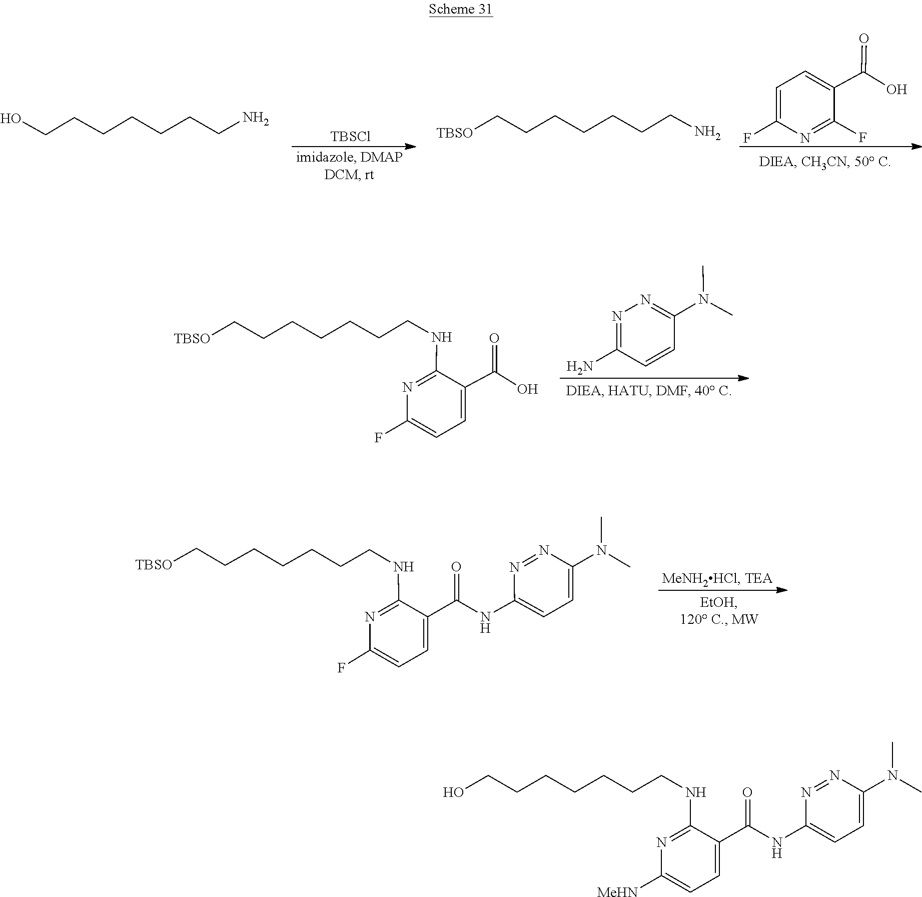

BL1-31 was synthesized following the procedures for preparing BL1-26 (149 mg, 3% yield over 4 steps) as a grey solid. ¹HNMR (400 MHz, MeOH-d₄) δ 7.99 (d, J=9.6 Hz, 1H), 7.77 (d, J=8.8 Hz, 1H), 7.19 (d, J=10.0 Hz, 1H), 5.74 (d, J=8.8 Hz, 1H), 3.54 (t, J=6.8 Hz, 2H), 3.49 (t, J=6.8 Hz, 2H), 3.13 (s, 6H), 2.92 (s, 3H), 1.67-1.61 (m, 2H), 1.56-1.51 (m, 2H), 1.42-1.38 (m, 6H). MS (ESI) m/z=402.1 [M+H]⁺.

### Example 32. N-(6-(dimethylamino)pyridazin-3-yl)-2-((8-hydroxyoctyl)amino)-6-(methylamino)nicotinamide (BL1-32)

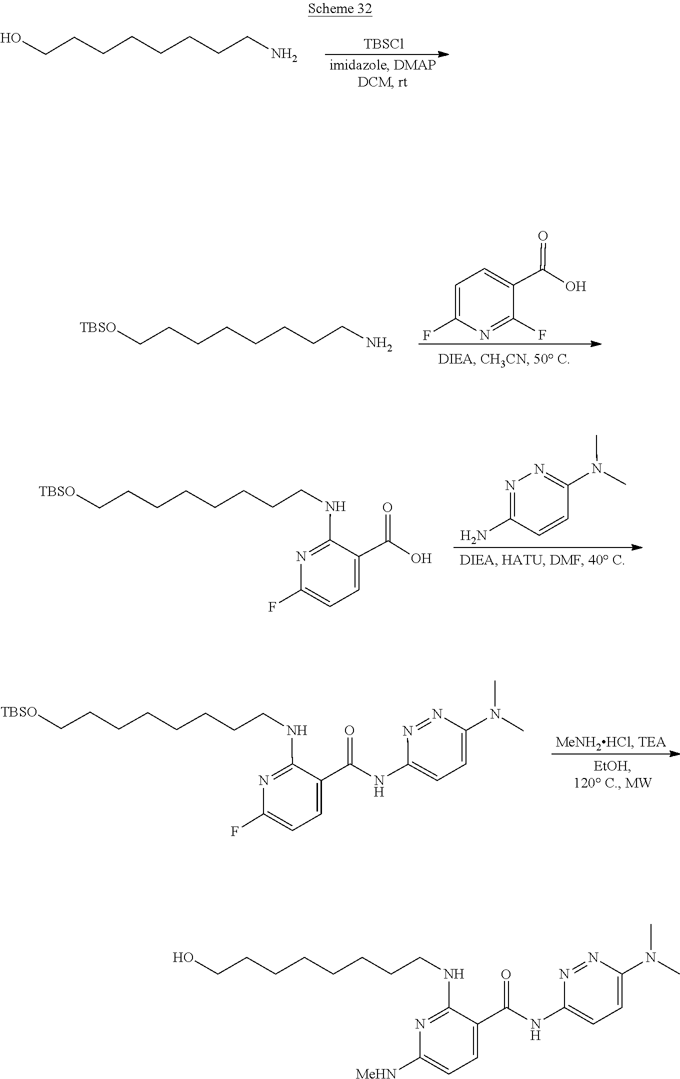

BL1-32 was synthesized following the procedures for preparing BL1-26 (411 mg, 3% yield over 4 steps) as a green solid. ¹HNMR (400 MHz, CDCl₃) δ 8.53 (s, 1H), 8.16 (d, J=10.0 Hz, 1H), 7.65 (d, J=8.8 Hz, 1H), 6.90 (d, J=10.0 Hz, 1H), 5.67 (d, J=8.8 Hz, 1H), 4.86 (s, 1H), 3.69-3.61 (m, 2H), 3.53-3.43 (m, 2H), 3.15 (s, 6H), 2.96 (d, J=4.4 Hz, 3H), 1.73-1.49 (m, 6H), 1.46-1.29 (m, 6H). MS (ESI) m/z=416.2 [M+H]⁺.

### Example 33. N-(6-(dimethylamino)pyridazin-3-yl)-2-((2-(2-hydroxyethoxy)ethyl)amino)-6-(methylamino)nicotinamide (BL1-33)

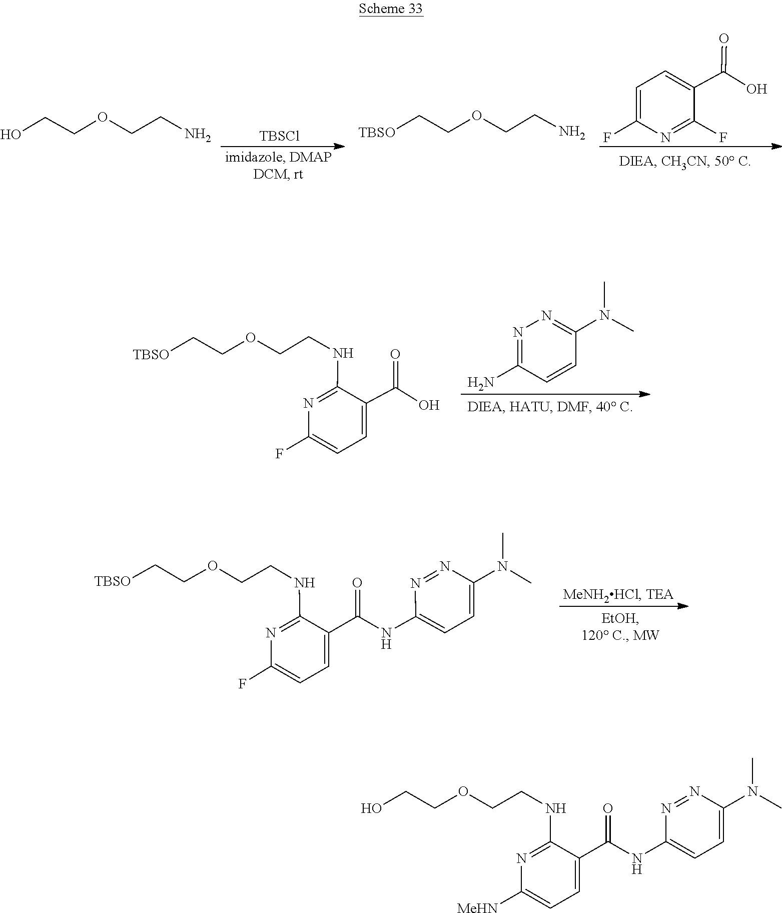

BL1-33 was synthesized following the procedures for preparing BL1-26 (212 mg, 3% yield over 4 steps) as a green solid. ¹HNMR (400 MHz, MeOH-d₄) δ 7.99 (d, J=10.0 Hz, 1H), 7.78 (d, J=8.4 Hz, 1H), 7.18 (d, J=9.6 Hz, 1H), 5.77 (d, J=8.8 Hz, 1H), 3.71-3.65 (m, 6H), 3.58 (t, J=4.4 Hz, 2H), 3.13 (s, 6H), 2.91 (s, 3H). MS (ESI) m/z=376.0 [M+H]⁺.

### Example 34. N-(6-(dimethylamino)pyridazin-3-yl)-2-((2-(2-(2-hydroxyethoxy)ethoxy)ethyl)amino)-6-(methylamino)nicotinamide (BL1-34)

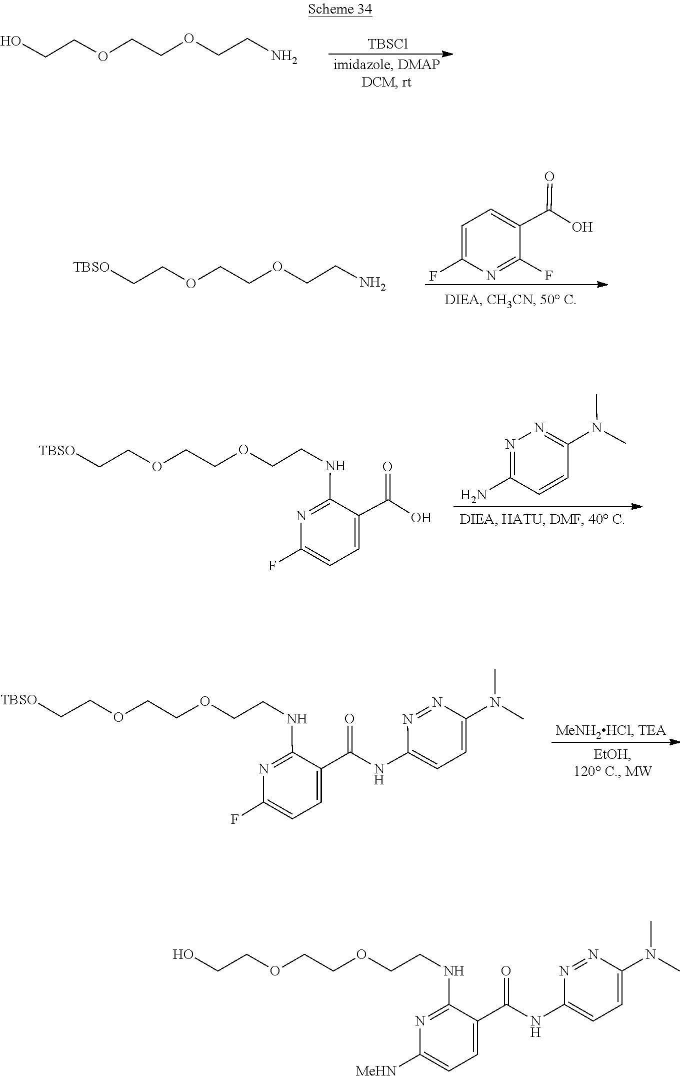

BL1-34 was synthesized following the procedures for preparing BL1-26 (122 mg, 2% yield over 4 steps) as a gray solid. ¹HNMR (400 MHz, MeOH-d₄) δ 8.00 (d, J=9.6 Hz, 1H), 7.78 (d, J=8.8 Hz, 1H), 7.18 (d, J=10.0 Hz, 1H), 5.76 (d, J=8.8 Hz, 1H), 3.70 (s, 4H), 3.68-3.62 (m, 6H), 3.60-3.54 (m, 2H), 3.13 (s, 6H), 2.91 (s, 3H). MS (ESI) m/z=420.3 [M+H]⁺.

### Example 35. N-(6-(dimethylamino)pyridazin-3-yl)-2-((2-(2-(2-(2-hydroxyethoxy)ethoxy)ethoxy)ethyl)amino)-6-(methylamino)nicotinamide (BL1-35)

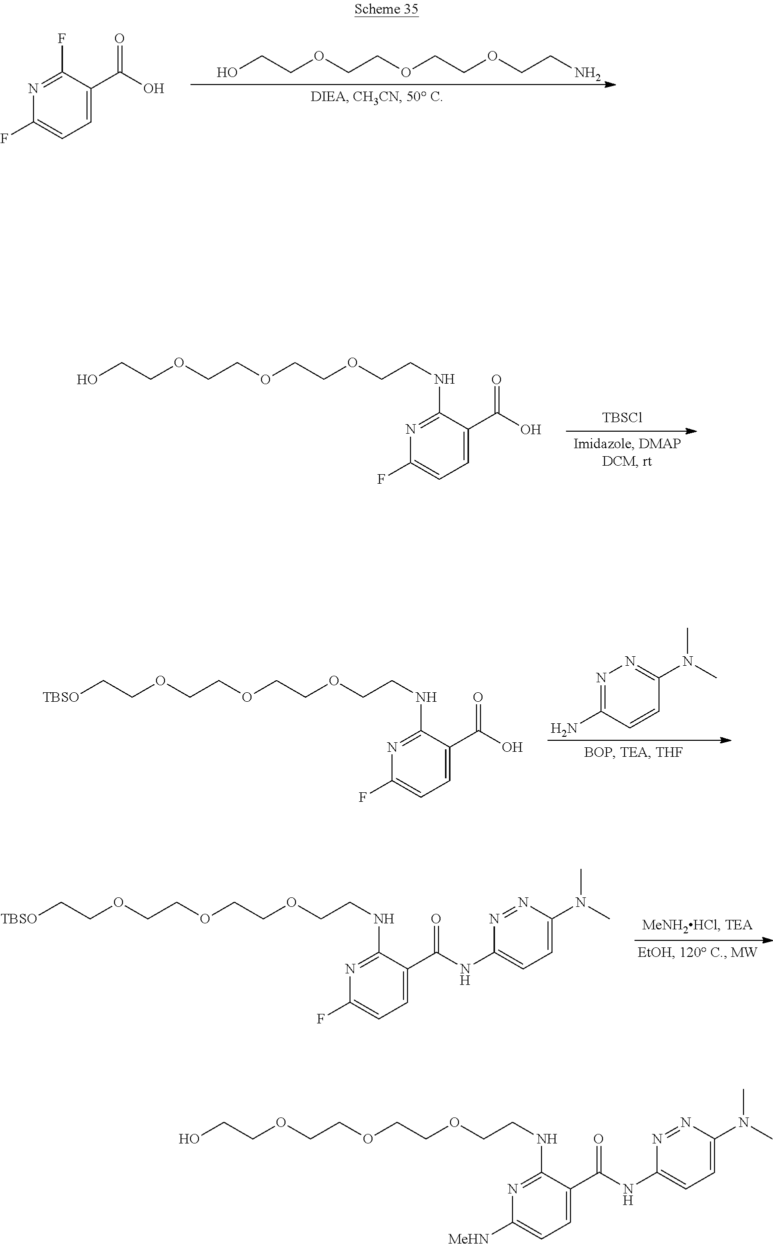

### Step 1. Synthesis of 6-fluoro-2-[2-[2-[2-(2-hydroxyethoxy)ethoxy]ethoxy]ethylamino]pyridine-3-carboxylic acid

The title compound was synthesized following the procedure of step 2 for preparing BL1-26 to provide the desired product (1.25 g, 73% yield) as a yellow oil. MS (ESI) m/z=333.3 [M+H]⁺.

### Step 2. Synthesis of 2-[2-[2-[2-[2-[tert-butyl(dimethyl)silyl]oxyethoxy]ethoxy]ethoxy]ethylamino]-6-fluoro-pyridine-3-carboxylic acid

The title compound was synthesized following the procedure of step 1 for preparing BL1-26 to provide the desired product (1.27 g, 76% yield) as a yellow oil. MS (ESI) m/z=447.2 [M+H]⁺.

### Step 3. Synthesis of N-(6-(dimethylamino)pyridazin-3-yl)-6-fluoro-2-((2,2,3,3-tetramethyl-4,7,10,13-tetraoxa-3-silapentadecan-15-yl)amino)nicotinamide

The title compound was synthesized following the procedure of step 3 for preparing BL1-26 to provide the desired product (200 mg, 16% yield) as a yellow solid. MS (ESI) m/z=453.3 [M+H]⁺.

### Step 4. Synthesis of N-[6-(dimethylamino)pyridazin-3-yl]-2-[2-[2-[2-(2-hydroxyethoxy)ethoxy]ethoxy]ethylamino]-6-(methylamino)pyridine-3-carboxamide

The title compound was synthesized following the procedure of step 4 for preparing BL1-268 to provide the desired product (167 mg, 78% yield) as a brown gum. ¹HNMR (400 MHz, MeOH-d4) δ 7.99 (d, J=9.6 Hz, 1H), 7.77 (d, J=8.4 Hz, 1H), 7.19 (d, J=10.0 Hz, 1H), 5.76 (d, J=8.8 Hz, 1H), 3.69 (s, 4H), 3.66-3.63 (m, 6H), 3.63-3.57 (m, 4H), 3.53-3.50 (m, 2H), 3.13 (s, 6H), 2.91 (s, 3H). MS (ESI) m/z=464.3 [M+H]⁺.

### Example 36. N-(6-(dimethylamino)pyridazin-3-yl)-2-((14-hydroxy-3,6,9,12-tetraoxatetradecyl)amino)-6-(methylamino)nicotinamide (BL1-36)

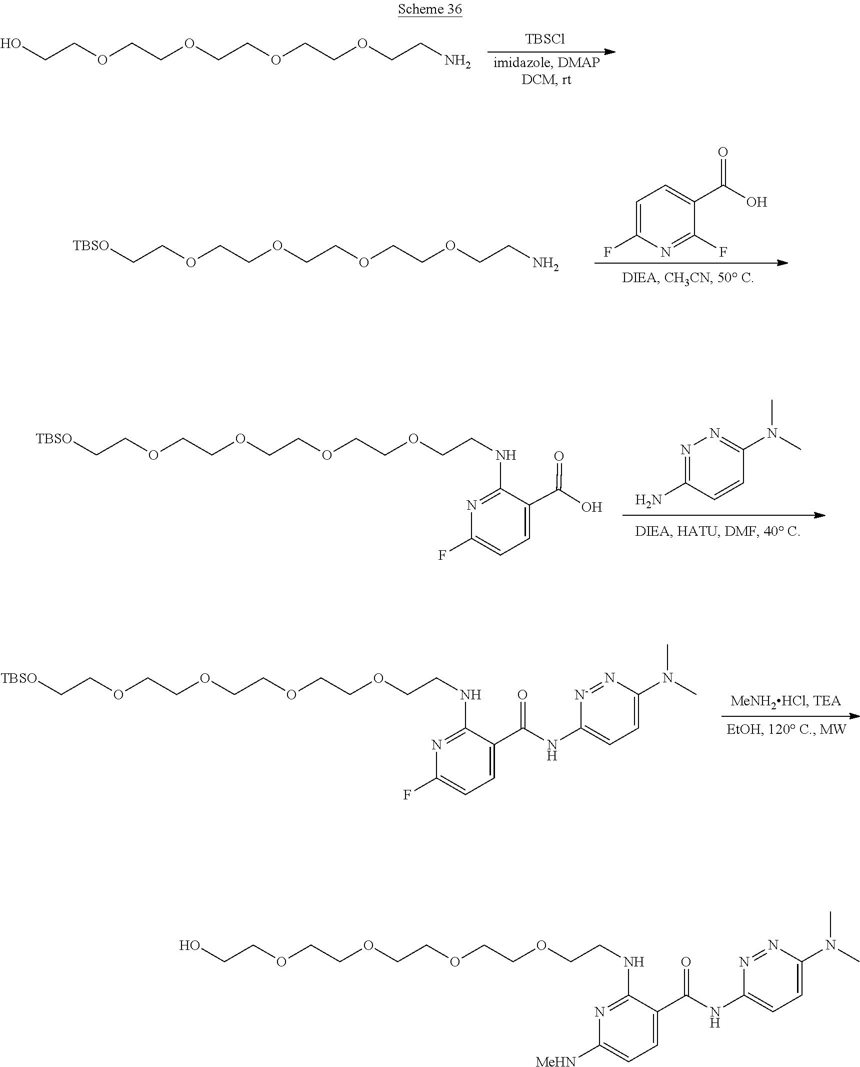

BL1-36 was synthesized following the procedures for preparing BL1-26 (212 mg, 5% yield over 4 steps) as a yellow gum. ¹HNMR (400 MHz, MeOH-d₄) δ 8.00 (d, J=10.0 Hz, 1H), 7.78 (d, J=8.8 Hz, 1H), 7.19 (d, J=9.6 Hz, 1H), 5.77 (d, J=8.8 Hz, 1H), 3.69 (s, 4H), 3.66-3.62 (m, 8H), 3.62-3.58 (m, 6H), 3.53 (t, J=5.2 Hz, 2H), 3.13 (s, 6H), 2.91 (s, 3H). MS (ESI) m/z=508.4 [M+H]⁺.

### Example 37. tert-butyl (2-((3-((6-(dimethylamino)pyridazin-3-yl)carbamoyl)-6-(methylamino)pyridin-2-yl)amino)ethyl)carbamate (BL1-37)

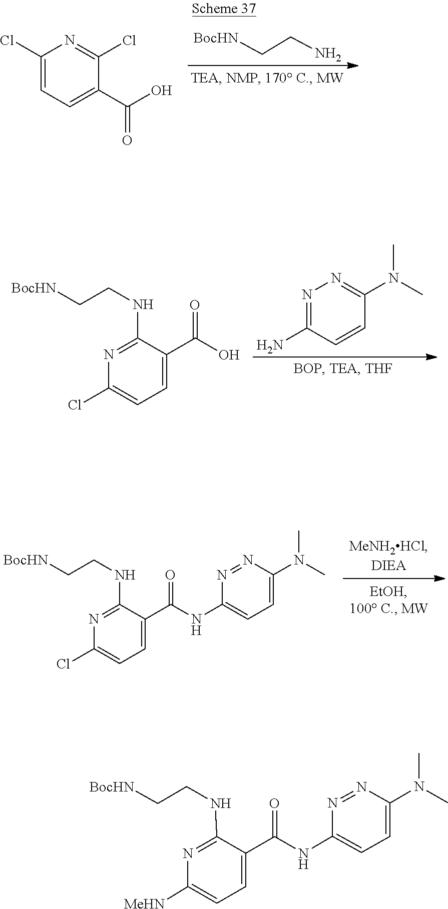

### Step 1. Synthesis of 2-[2-(tert-butoxycarbonylamino)ethylamino]-6-chloro-pyridine-3-carboxylic acid

To a solution of 2,6-dichloropyridine-3-carboxylic acid (0.50 g, 2.60 mmol) and tert-butyl N-(2-aminoethyl) carbamate (420 mg, 2.60 mmol) in NMP (3 mL) was added TEA (800 mg, 7.90 mmol) at rt under N₂. The reaction mixture was stirred at 170 [symbol] under N₂ by microwave irradiation for 2 h. After cooling down to rt, the reaction mixture was purified by reversed-phase chromatography to provide the desired product (0.44 g, 53% yield) as a brown solid. MS (ESI) m/z=260.1 [M+H-56]⁺

### Step 2. Synthesis of tert- butyl N-[2-[[6-chloro-3-[[6-(dimethylamino)pyridazin-3-yl]carbamoyl]-2-pyridyl]amino]ethyl]carbamate

To a solution of 2-[2-(tert-butoxycarbonylamino)ethylamino]-6-chloro-pyridine-3-carboxylic acid (0.44 g, 1.40 mmol), TEA (427 mg, 4.2 mmol) and BOP (609 mg, 1.40 mmol) in THF (8 mL) was added N³,N³-dimethylpyridazine-3,6-diamine (212 mg, 1.50 mmol). The mixture was stirred at rt for 12 h, before it was quenched with water (30 mL) and extracted with ethyl acetate (30 mL×3). The combined organic layers were washed with brine (30 mL), dried over anhydrous sodium sulfate, filtered, and concentrated. The crude product was purified by silica gel column chromatography (petroleum ether/ethyl acetate=20:1 to 0:1) to provide the desired product (0.35 g, 59% yield) as a yellow oil. MS (ESI) m/z=436.0 [M+H]⁺

### Step 3. Synthesis of tert -butyl N-[2-[[3-[[6-(dimethylamino)pyridazin-3-yl]carbamoyl]-6-(methylamino)-2-pyridyl]amino]ethyl]carbamate

To a solution of tert-butyl N-[2-[[6-chloro-3-[[6-(dimethylamino)pyridazin-3-yl]carbamoyl]-2-pyridyl]amino]ethyl]carbamate (0.35 g, 0.81 mmol) and methanamine hydrochloride (1.10 g, 16.2 mmol) in EtOH (10 mL) was added DIEA (1.00 g, 8.10 mmol). The reaction mixture was stirred at 120 [symbol] under microwave irradiation for 3 h. After cooling down to rt, the reaction mixture was concentrated under vacuum. The crude product was purified by silica gel column chromatography (dichloromethane/methanol=20:1 to 0:1) to provide the desired product (0.18 g, 47% yield) as a yellow solid. ¹HNMR (400 MHz, MeOH-d₄) b 8.00 (d, J=10.0 Hz, 1H), 7.77 (d, J=8.8 Hz, 1H), 7.19 (d, J=9.6 Hz, 1H), 5.78 (d, J=8.8 Hz, 1H), 3.62-3.57 (m, 2H), 3.30-3.25 (m, 2H), 3.13 (s, 6H), 2.93 (s, 3H), 1.41 (s, 9H). MS (ESI) m/z=431.4 [M+H]⁺,

### Example 38. tert-butyl (3-((3-((6-(dimethylamino)pyridazin-3-yl)carbamoyl)-6-(methylamino)pyridin-2-yl)amino)propyl)carbamate (BL1-38)

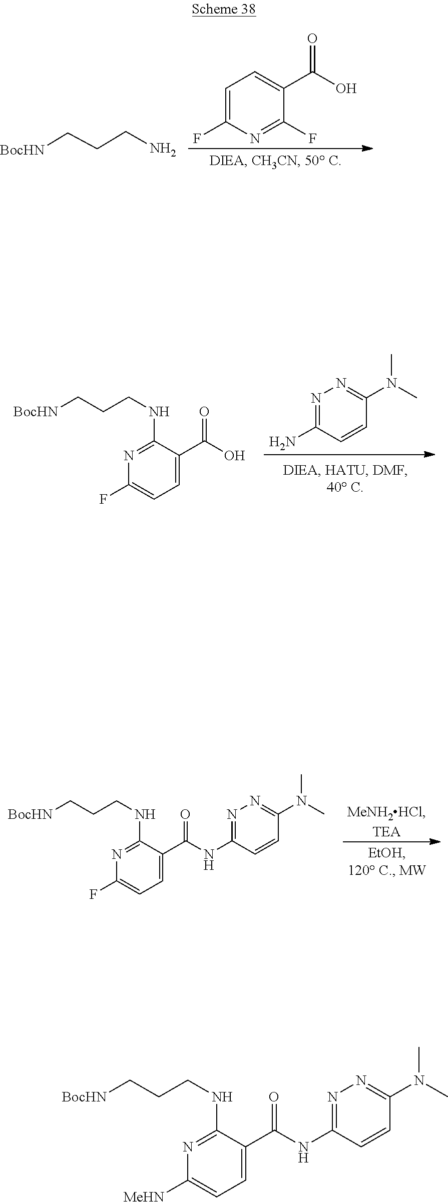

BL1-40 was synthesized following the procedures for preparing BL1-26 (160 mg, 6% yield over 3 steps) as a green solid. 1HNMR (400 MHz, MeOH-d₄) δ 8.01 (d, J=9.6 Hz, 1H), 7.77 (d, J=8.8 Hz, 1H), 7.18 (d, J=10.0 Hz, 1H), 5.76 (d, J=8.8 Hz, 1H), 3.74-3.72 (m, 2H), 3.55 (t, J=6.8 Hz, 2H), 3.13 (s, 6H), 2.93 (s, 3H), 1.88-1.86 (m, 2H), 1.78 (t, J=6.8 Hz, 2H), 1.43 (s, 9H). MS (ESI) m/z=445.5 [M+H]⁺

### Example 39. tert-butyl (4-((3-((6-(dimethylamino)pyridazin-3-yl)carbamoyl)-6-(methylamino)pyridin-2-yl)amino)butyl)carbamate (BL1-39)

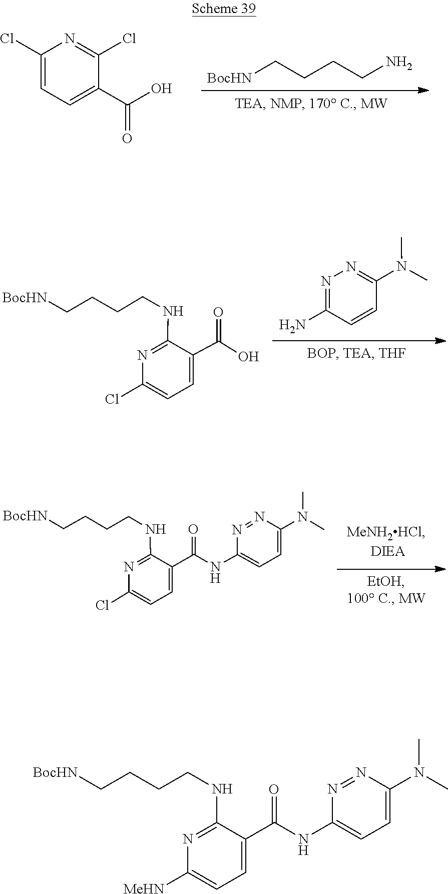

BL1-39 was synthesized following the procedures for preparing BL1-37 (120 mg, 10% yield over 3 steps) as a yellow solid. ¹HNMR (400 MHz, MeOH-d₄) δ 8.00 (d, J=10.0 Hz, 1H), 7.77 (d, J=8.8 Hz, 1H), 7.17 (d, J=9.6 Hz, 1H), 5.74 (d, J=8.4 Hz, 1H), 3.51 (t, J=6.4 Hz, 2H), 3.13 (s, 6H), 3.08 (t, J=5.6 Hz, 2H), 2.91 (s, 3H), 1.70-1.61 (m, 2H), 1.60-1.53 (m, 2H), 1.42 (s, 9H). MS (ESI) m/z=459.4 [M+H]⁺.

### Example 40. tert-butyl (6-((3-((6-(dimethylamino)pyridazin-3-yl)carbamoyl)-6-(methylamino)pyridin-2-yl)amino)hexyl)carbamate (BL1-40)

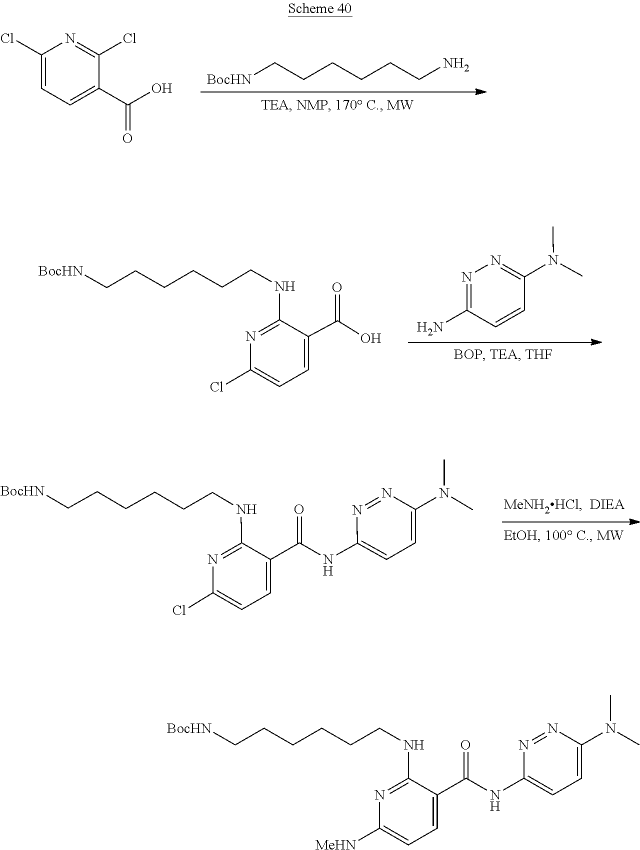

BL1-40 was synthesized following the procedures for preparing BL1-37 (140 mg, 11% yield over 3 steps) as a yellow solid. ¹HNMR (400 MHz, MeOH-d₄) (8.00 (d, J=9.6 Hz, 1H), 7.77 (d, J=8.8 Hz, 1H), 7.19 (d, J=10.0 Hz, 1H), 5.74 (d, J=8.8 Hz, 1H), 3.49 (t, J=6.8 Hz, 2H), 3.13 (s, 6H), 3.07-2.97 (m, 2H), 2.91 (s, 3H), 1.68-1.62 (m, 2H), 1.52-1.46 (m, 2H), 1.42 (s, 9H), 1.39-1.34 (m, 4H). MS (ESI) m/z=487.4 [M+H]⁺.

### Example 41. tert-butyl (8-((3-((6-(dimethylamino)pyridazin-3-yl)carbamoyl)-6-(methylamino)pyridin-2-yl)amino)octyl)carbamate (BL1-41)

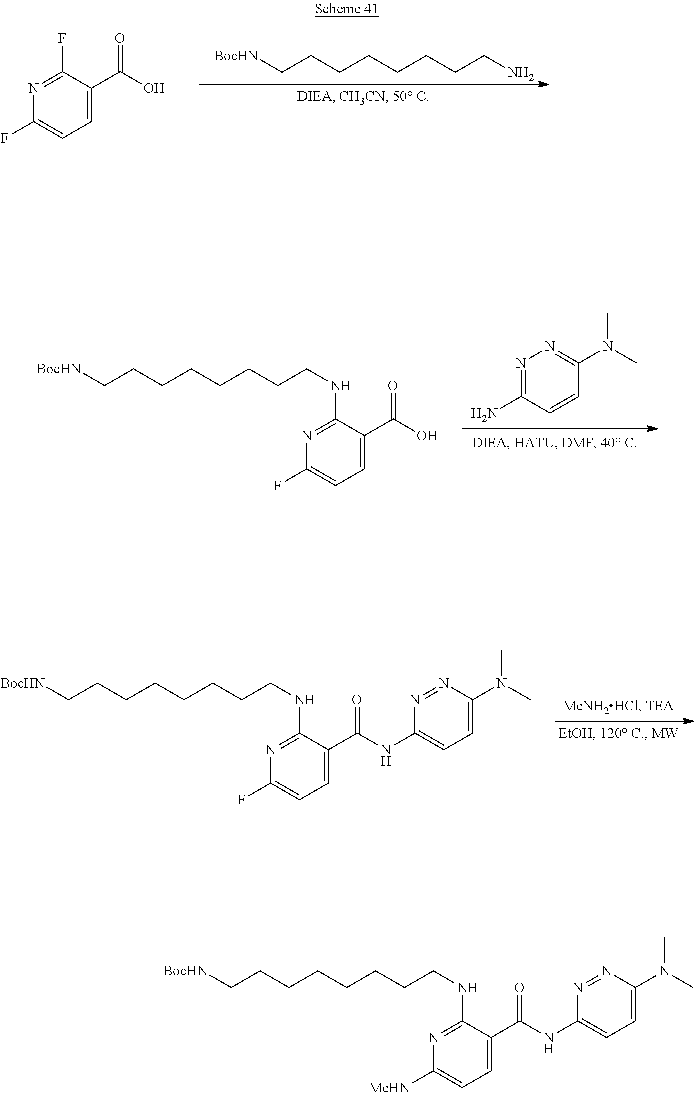

BL1-41 was synthesized following the procedures for preparing BL1-26 (386 mg, 20% yield over 3 steps) as a yellow solid. MS (ESI) m/z=515.4 [M+H]⁺.

### Example 42. tert-butyl (2-(2-((3-((6-(dimethylamino)pyridazin-3-yl)carbamoyl)-6-(methylamino)pyridin-2-yl)amino)ethoxy)ethyl)carbamate (BL1-42)

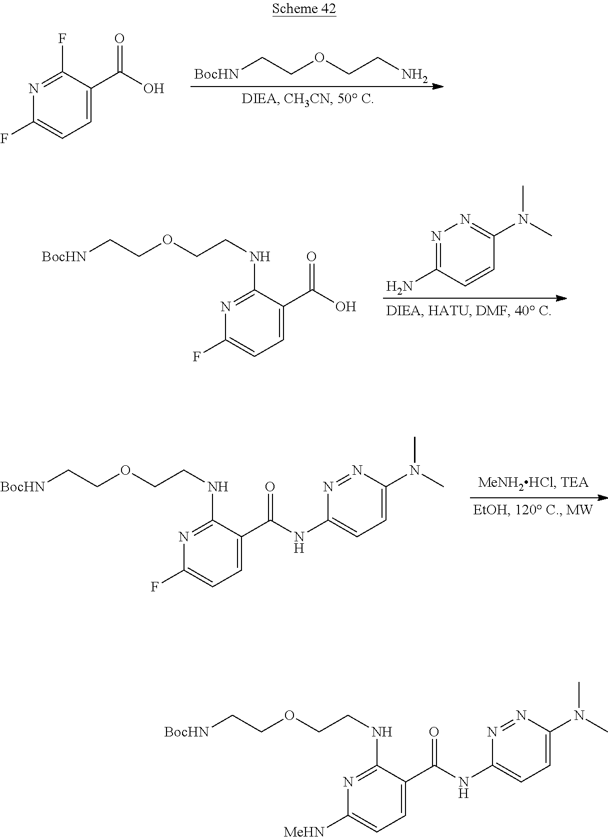

BL1-42 was synthesized following the procedures for preparing BL1-26 (574 mg, 17% yield over 3 steps) as a brown solid. ¹′HNMR (400 MHz, MeOH-d₄) δ 8.02 (d, J=10.0 Hz, 1H), 7.78 (d, J=8.8 Hz, 1H), 7.18 (d, J=8.8 Hz, 1H), 5.77 (d, J=8.4 Hz, 1H), 3.71-3.63 (m, 4H), 3.53 (t, J=5.6 Hz, 2H), 3.23 (t, J=5.6 Hz, 2H), 3.13 (s, 6H), 2.91 (s, 3H), 1.38 (s, 9H). MS (ESI) m/z=475.4 [M+H]⁺.

### Example 43. tert-butyl (2-(2-(2-((3-((6-(dimethylamino)pyridazin-3-yl)carbamoyl)-6-(methylamino)pyridin-2-yl)amino)ethoxy)ethoxy)ethyl)carbamate (BL1-43)

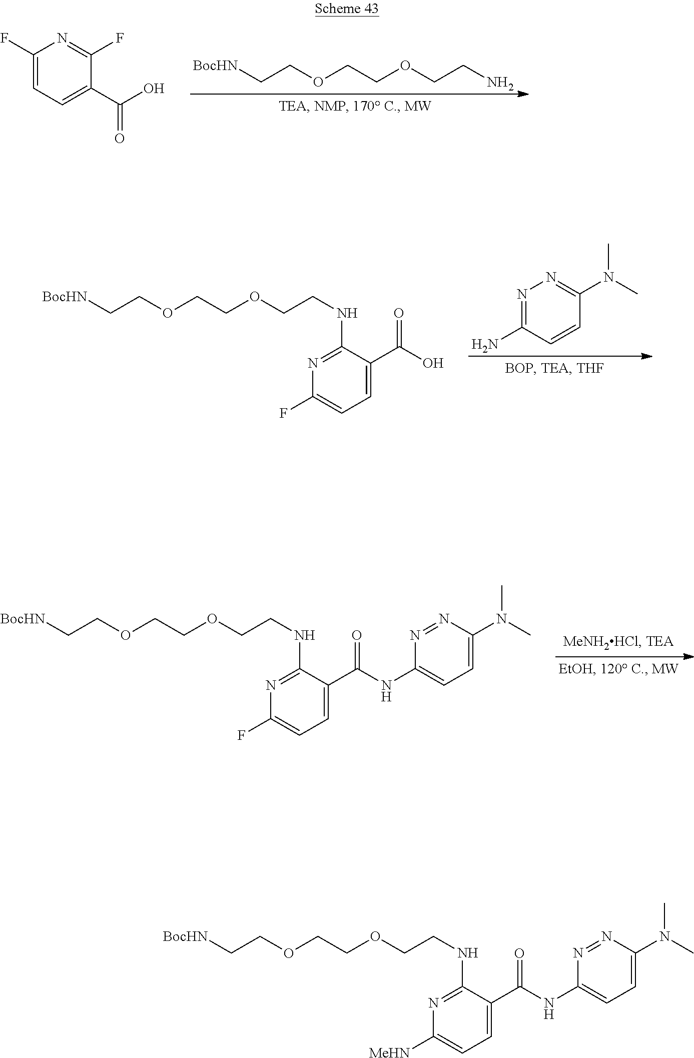

BL1-43 was synthesized following the procedures for preparing BL1-37 (263 mg, 27% yield over 3 steps) as a yellow solid. ¹HNMR (400 MHz, MeOH-d₄) δ 8.00 (brd, J=9.6 Hz, 1H), 7.78 (d, J=8.8 Hz, 1H), 7.19 (d, J=9.6 Hz, 1H), 5.77 (d, J=8.8 Hz, 1H), 3.70 (s, 4H), 3.67-3.60 (m, 4H), 3.52 (t, J=5.6 Hz, 2H), 3.20 (t, J=5.6 Hz, 2H), 3.13 (s, 6H), 2.91 (s, 3H), 1.40 (s, 9H). MS (ESI) m/z=519.3 [M+H]⁺.

### Example 44. tert-butyl (2-(2-(2-(2-((3-((6-(dimethylamino)pyridazin-3-yl)carbamoyl)-6-(methylamino)pyridin-2-yl)amino)ethoxy)ethoxy)ethoxy)ethyl)carbamate (BL1-44)

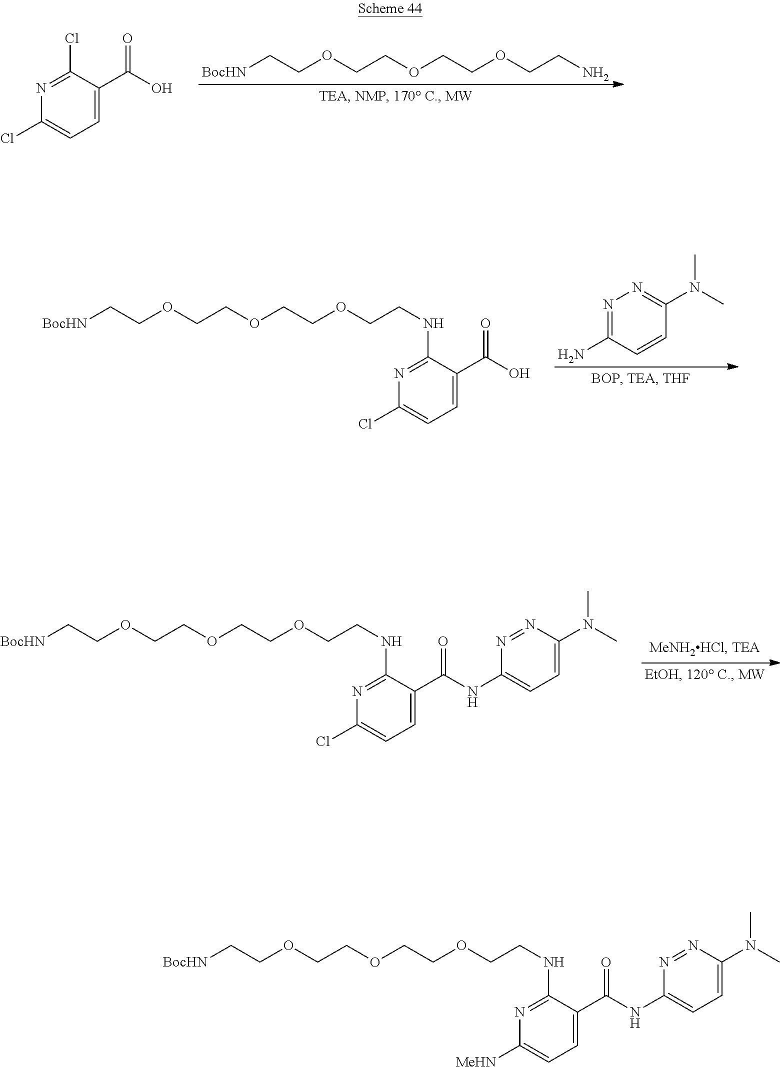

BL1-44 was synthesized following the procedures for preparing BL1-37 (166 mg, 11% yield over 3 steps) as a yellow gum. ¹HNMR (400 MHz, MeOH-d₄) δ 8.01 (d, J=10.0 Hz, 1H), 7.78 (d, J=8.8 Hz, 1H), 7.18 (d, J=10.0 Hz, 1H), 5.76 (d, J=8.8 Hz, 1H), 3.70 (s, 4H), 3.68-3.62 (m, 6H), 3.59-3.54 (m, 2H), 3.46 (t, J=5.2 Hz, 2H), 3.18 (t, J=5.6 Hz, 2H), 3.13 (s, 6H), 2.91 (s, 3H), 1.42 (s, 9H). MS (ESI) m/z=563.5 [M+H].

### Example 45. tert-butyl (14-((3-((6-(dimethylamino)pyridazin-3-yl)carbamoyl)-6-(methylamino)pyridin-2-yl)amino)-3,6,9,12-tetraoxatetradecyl)carbamate (BL1-45)

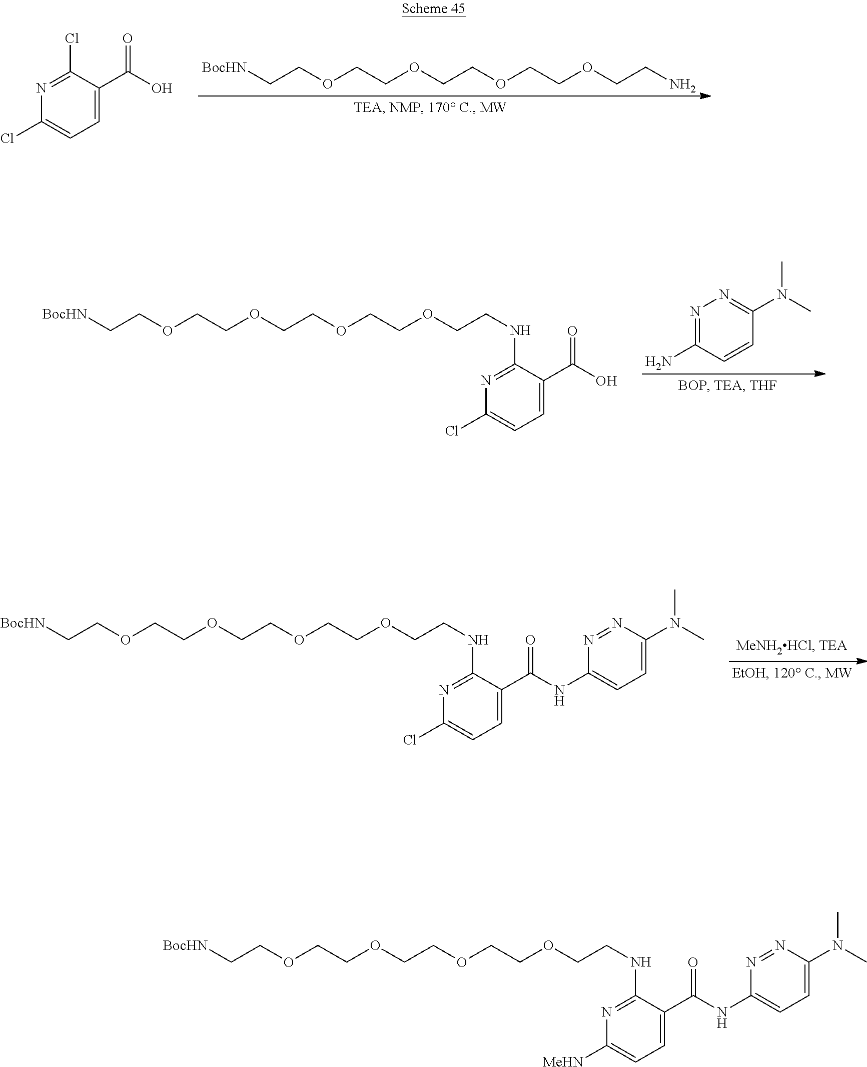

BL1-45 was synthesized following the procedures for preparing BL1-37 (110 mg, 7% yield over 3 steps) as a yellow oil. MS (ESI) m/z=607.4 [M+H]⁺.

### Example 46. (3-((6-(dimethylamino)pyridazin-3-yl)carbamoyl)-6-(methylamino)pyridin-2-yl)glycine (BL1-46)

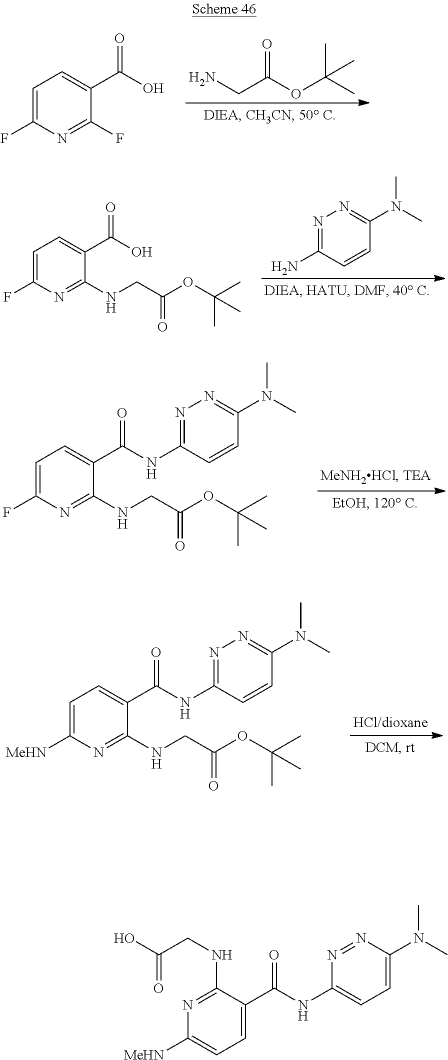

### Step 1. Synthesis of 2-((2-(tert-butoxy)-2-oxoethyl)amino)-6-fluoronicotinic acid

To a solution of 2,6-difluoropyridine-3-carboxylic acid (2.43 g, 15.3 mmol) in CH₃CN (20 mL) were added tert-butyl 2-aminoacetate (2.00 g, 15.3 mmol) and DIEA (5.91 g, 45.7 mmol) at rt. The reaction mixture was stirred at 50° C. for 12 h. Then the reaction mixture was diluted with citric acid (4 M, 20 mL) and extracted with dichloromethane (30 mL×3). The combined organic layers were washed with brine (30 mL), dried over anhydrous Na₂SO₄, filtered, and concentrated under reduced pressure. The residue was purified by silica gel column chromatography (dichloromethane/methanol=20:1) to provide the desired product (3.70 g, 89% yield) as a yellow oil. ¹HNMR (400 MHz, MeOH-d₄) 8.24 (t, J=8.4 Hz, 1H), 6.16-6.08 (m, 1H), 4.05 (s, 2H), 1.46 (s, 9H). MS (ESI) m/z=215.0 [M+H]⁺

### Step 2. Synthesis of 2-((3-((6-(dimethylamino)pyridazin-3-yl)carbamoyl)-6-fluoropyridin-2-yl)amino)acetic acid

To a solution of 2-[(2-tert-butoxy-2-oxo-ethyl)amino]-6-fluoro-pyridine-3-carboxylic acid (1.00 g, 3.70 mmol) in DMF (10 mL) were added HATU (1.41 g, 3.70 mmol), DIEA (1.43 g, 11.1 mmol) and N³,N³-dimethylpyridazine-3,6-diamine (512 mg, 3.71 mmol) at rt. After the reaction mixture was stirred at 40° C. for 5 h, it was poured into H₂O (15 mL) and extracted with ethyl acetate (10 mL×3). The combined organic layers were washed with brine (50 mL), dried over Na₂SO₄, filtered, and concentrated. The residue was purified by silica gel column chromatography (petroleum ether/ethyl acetate=1:1) to provide the desired product (1.10 g, 76% yield) as a yellow oil. MS (ESI) m/z=391.4 [M+H]⁺

### Step 3. Synthesis of 2-((3-((6-(dimethylamino)pyridazin-3-yl)carbamoyl)-6-(methylamino)pyridin-2-yl)amino)acetic acid

To a solution of tert-butyl 2-[[3-[[6-(dimethylamino)pyridazin-3-yl]carbamoyl]-6-fluoro-2-pyridyl]amino]acetate (1.00 g, 2.56 mmol) in EtOH (5 mL) was added TEA (2.58 g, 25.6 mmol) and methylamine hydrochloride (800 mg, 25.8 mmol) at rt. The mixture was stirred at 120 [symbol] for 4 h. After cooling down to rt, the reaction mixture was concentrated under vacuum. The residue was diluted with H₂O (15 mL) and extracted with ethyl acetate (30 mL×3). The combined organic layers were washed with brine (50 mL), dried over Na₂SO₄, filtered, and concentrated. The residue was purified by silica gel column chromatography (petroleum ether/ethyl acetate=0:1) to provide the desired product (120 mg, 12% yield) as a yellow solid. ¹HNMR (400 MHz, MeOH-d₄) δ 8.04 (d, J=9.6 Hz, 1H), 7.80 (d, J=8.8 Hz, 1H), 7.19 (d, J=10.0 Hz, 1H), 5.81 (d, J=8.8 Hz, 1H), 4.11 (s, 2H), 3.14 (s, 6H), 2.91 (s, 3H), 1.46 (s, 9H). MS (ESI) m/z=402.4 [M+H]⁺.

### Step 4. Synthesis of 2-((3-((6-(dimethylamino)pyridazin-3-yl)carbamoyl)-6-(methylamino)pyridin-2-yl)amino)acetic acid

To a solution of tert-butyl 2-[[3-[[6-(dimethylamino)pyridazin-3-yl]carbamoyl]-6-(methylamino)-2-pyridyl]amino]acetate (100 mg, 0.25 mmol) in DCM (1 mL) was added HCl solution (4 M in dioxane, 1 mL) at rt. After the mixture was stirred at rt for 12 h, it was concentrated under vacuum to provide the desired product (101 mg, 93% yield) as a green solid. ¹HNMR (400 MHz, D₂O) δ 8.23-8.04 (m, 2H), 7.72 (d, J=10.4 Hz, 1H), 6.09 (d, J=9.6 Hz, 1H), 4.22 (s, 2H), 3.28 (s, 6H), 2.95 (s, 3H). MS (ESI) m/z=346.4 [M+H]⁺.

### Example 47. 3-((3-((6-(dimethylamino)pyridazin-3-yl)carbamoyl)-6-(methylamino)pyridin-2-yl)amino)propanoic acid (BL1-47)

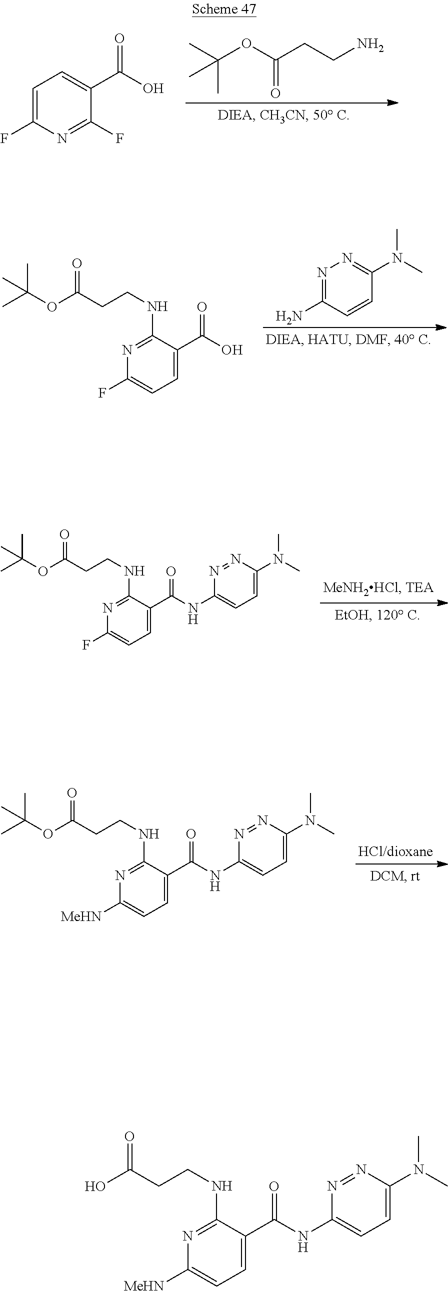

BL1-47 was synthesized following the procedures for preparing BL1-46 (159 mg, 13% yield over 4 steps) as an off-white solid. ¹HNMR (400 MHz, MeOH-d₄) δ 8.37 (d, J=10.0 Hz, 1H), 8.32-8.21 (m, 1H), 7.88 (d, J=10.0 Hz, 1H), 6.15 (d, J=9.2 Hz, 1H), 3.79 (t, J=5.6 Hz, 2H), 3.33 (s, 6H), 3.09 (s, 3H), 2.79 (t, J=6.0 Hz, 2H). MS (ESI) m/z=360.1 [M+H]⁺.

### Example 48. 4-((3-((6-(dimethylamino)pyridazin-3-yl)carbamoyl)-6-(methylamino)pyridin-2-yl)amino)butanoic acid (BL1-48)

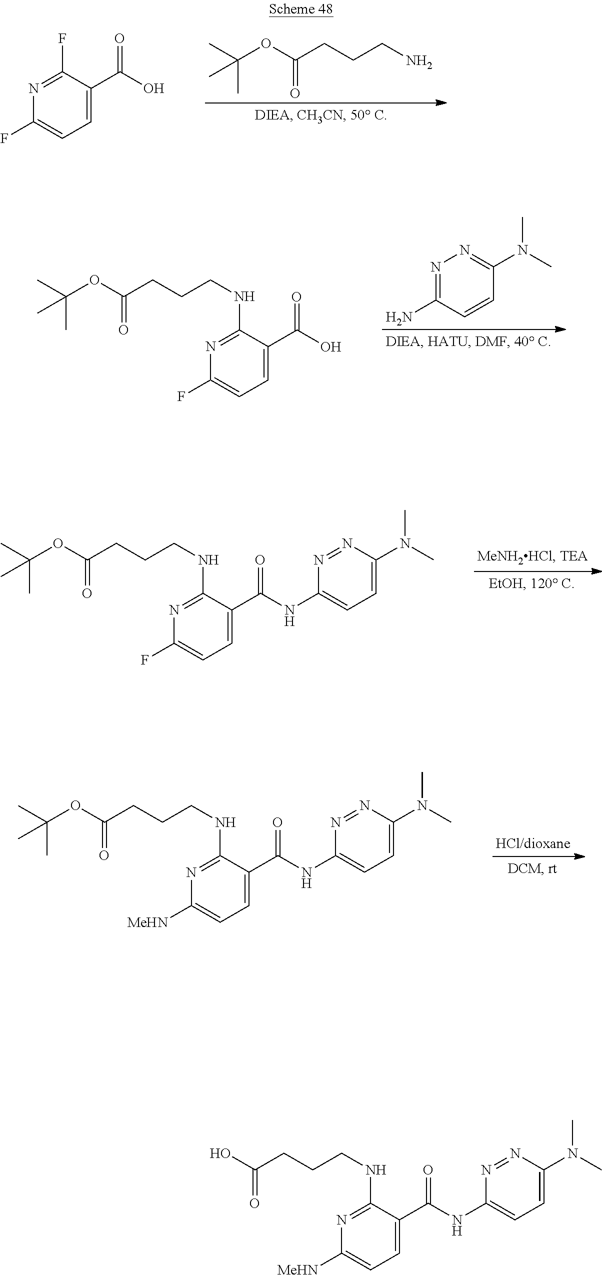

BL1-48 was synthesized following the procedures for preparing BL1-46 (226 mg, 23% yield over 4 steps) as a yellow solid. ¹HNMR (400 MHz, MeOH-d₄) δ 8.43-8.22 (m, 2H), 7.88 (d, J=10.4 Hz, 1H), 6.13 (d, J=9.2 Hz, 1H), 3.61-3.52 (m, 2H), 3.33 (s, 6H), 3.06 (s, 3H), 2.49 (t, J=6.8 Hz, 2H), 2.00 (m, J=7.2 Hz, 2H). MS (ESI) m/z- 374.4 [M+H]⁺.

### Example 49. 5-((3-((6-(dimethylamino)pyridazin-3-yl)carbamoyl)-6-(methylamino)pyridin-2-yl)amino)pentanoic acid (BL1-49)

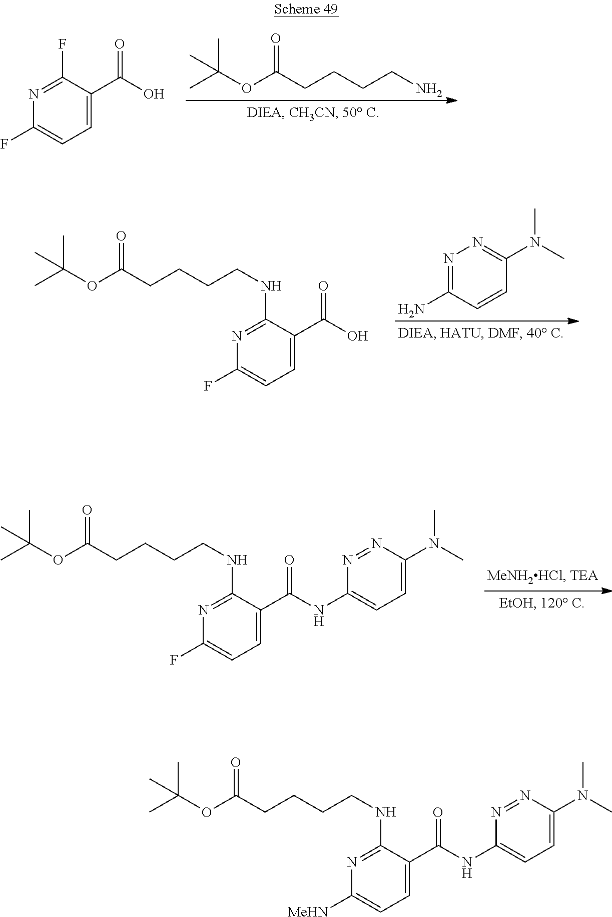

BL1-49 was synthesized following the procedures for preparing BL1-46 (109 mg, 7% yield over 3 steps) as a yellow solid. ¹HNMR (400 MHz, MeOH-d₄) b 8.01 (d, J=9.6 Hz, 1H), 7.77 (d, J=8.8 Hz, 1H), 7.18 (d, J=10.0 Hz, 1H), 5.75 (d, J=8.8 Hz, 1H), 3.52 (t, J=5.6 Hz, 2H), 3.14 (s, 6H), 2.92 (s, 3H), 2.28 (t, J=6.8 Hz, 2H), 1.70-1.62 (m, 4H), 1.43 (s, 9H). MS (ESI) m/z=444.2 [M+H]⁺.

### Example 50. 6-((3-((6-(dimethylamino)pyridazin-3-yl)carbamoyl)-6-(methylamino)pyridin-2-yl)amino)hexanoic acid (BL1-50)

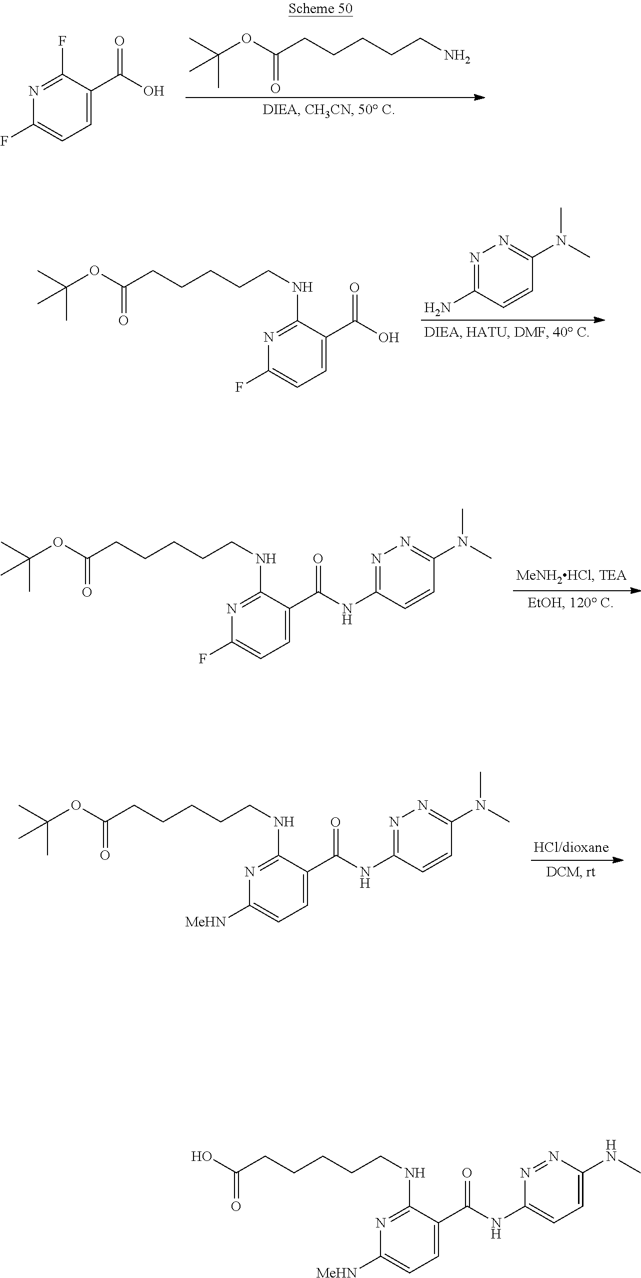

BL1-50 was synthesized following the procedures for preparing BL1-46 (131 mg, 19% yield over 4 steps) as a yellow solid. ¹HNMR (400 MHz, MeOH-d₄) b 8.38-8.30 (m, 2H), 7.88 (d, J=10.4 Hz, 1H), 6.11 (d, J=9.2 Hz, 1H), 3.53 (t, J=7.2 Hz, 2H), 3.33 (s, 6H), 3.04 (s, 3H), 2.33 (t, J=7.6 Hz, 2H), 1.80-1.69 (m, 4H), 1.67-1.41 (m, 2H). MS (ESI) m/z=402.3 [M+H]⁺.

### Example 51. 7-((3-((6-(dimethylamino)pyridazin-3-yl)carbamoyl)-6-(methylamino)pyridin-2-yl)amino)heptanoic acid (BL1-51)

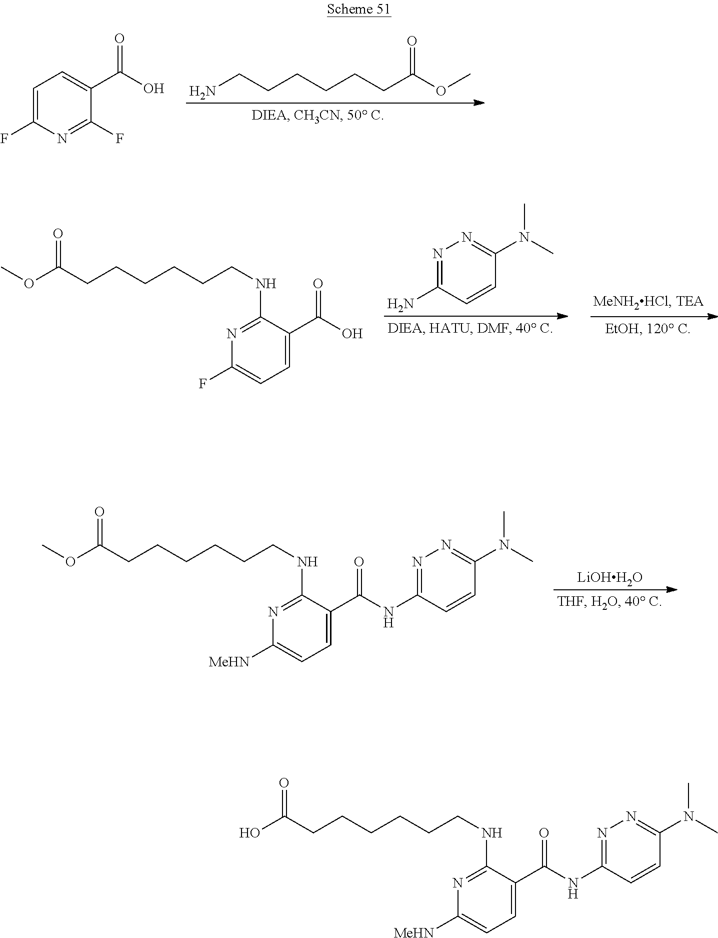

### Step 1. Synthesis of 6-fluoro-2-((7-methoxy-7-oxoheptyl)amino)nicotinic acid

The title compound was synthesized following the procedure of step 1 for preparing BL1-46 to provide the desired product (1.60 g, 52% yield) as a white solid. ¹HNMR (400 MHz, MeOH-d4) δ 8.24 (t, J=8.4 Hz, 1H), 6.10 (dd, J=2.8 Hz, 8.4 Hz, 1H), 3.65 (s, 3H), 3.43 (t, J=6.8 Hz, 2H), 2.33 (t, J=7.6 Hz, 2H), 1.67-1.58 (m, 4H), 1.44-1.37 (m, 4H). MS (ESI) m/z=299.2 [M+H]⁺.

### Step 2. Synthesis of methyl 7-((3-((6-(dimethylamino)pyridazin-3-yl)carbamoyl)-6-fluoropyridin-2-yl)amino)heptanoate

The title compound was synthesized following the procedure of step 2 for preparing BL1-46 to provide the desired product (1.00 g, 33% yield) as a yellow solid. MS (ESI) m/z=445.5 [M+H]⁺.

### Step 3. Synthesis of methyl 7-((3-((6-(dimethylamino)pyridazin-3-yl)carbamoyl)-6-(methylamino)pyridin-2-yl)amino)heptanoate

The title compound was synthesized following the procedure of step 3 for preparing BL1-46 to provide the desired product (110 mg, 11% yield) as a green solid. ¹HNMR (400 MHz, MeOH-d₄) δ 8.00 (d, J=9.6 Hz, 1H), 7.77 (d, J=8.8 Hz, 1H), 7.18 (d, J=9.6 Hz, 1H), 5.74 (d, J=8.8 Hz, 1H), 3.64 (s, 3H), 3.45-3.33 (t, J=6.8 Hz, 2H), 3.14 (s, 6H), 2.91 (s, 3H), 2.34-2.29 (m, 2H), 1.64-1.54 (m, 4H), 1.44-1.39 (m, 4H). MS (ESI) m/z=430.2 [M+H]⁺.

### Step 4. Synthesis of 8-((3-((6-(dimethylamino)pyridazin-3-yl)carbamoyl)-6-(methylamino)pyridin-2-yl)amino)octanoic acid

To a solution of methyl 7-[[3-[[6-(dimethylamino)pyridazin-3-yl]carbamoyl]-6-(methylamino)-2-pyridyl]amino]heptanoate (90 mg, 0.21 mmol) in H₂O (1 mL) and THF (1 mL) was added LiOH·H₂O (18.0 mg, 0.75 mmol) at rt. The reaction mixture was stirred at 40 [symbol] for 3 h, before it was concentrated and purified by prep-HPLC to provide the desired product (52.9 mg, 59% yield) as a yellow solid. ¹HNMR (400 MHz, MeOH-d₄) δ 8.01 (d, J=9.6 Hz, 1H), 7.77 (d, J=8.8 Hz, 1H), 7.19 (d, J=10.0 Hz, 1H), 5.75 (d, J=8.8 Hz, 1H), 3.50 (t, J=6.8 Hz, 2H), 3.14 (s, 6H), 2.92 (s, 3H), 2.28 (t, J=7.2 Hz, 2H), 1.68-1.60 (m, 4H), 1.47-1.39 (m, 4H). MS (ESI) m/z=416.4 [M+H]⁺.

### Example 52. 8-((3-((6-(dimethylamino)pyridazin-3-yl)carbamoyl)-6-(methylamino)pyridin-2-yl)amino)octanoic acid (BL1-52)

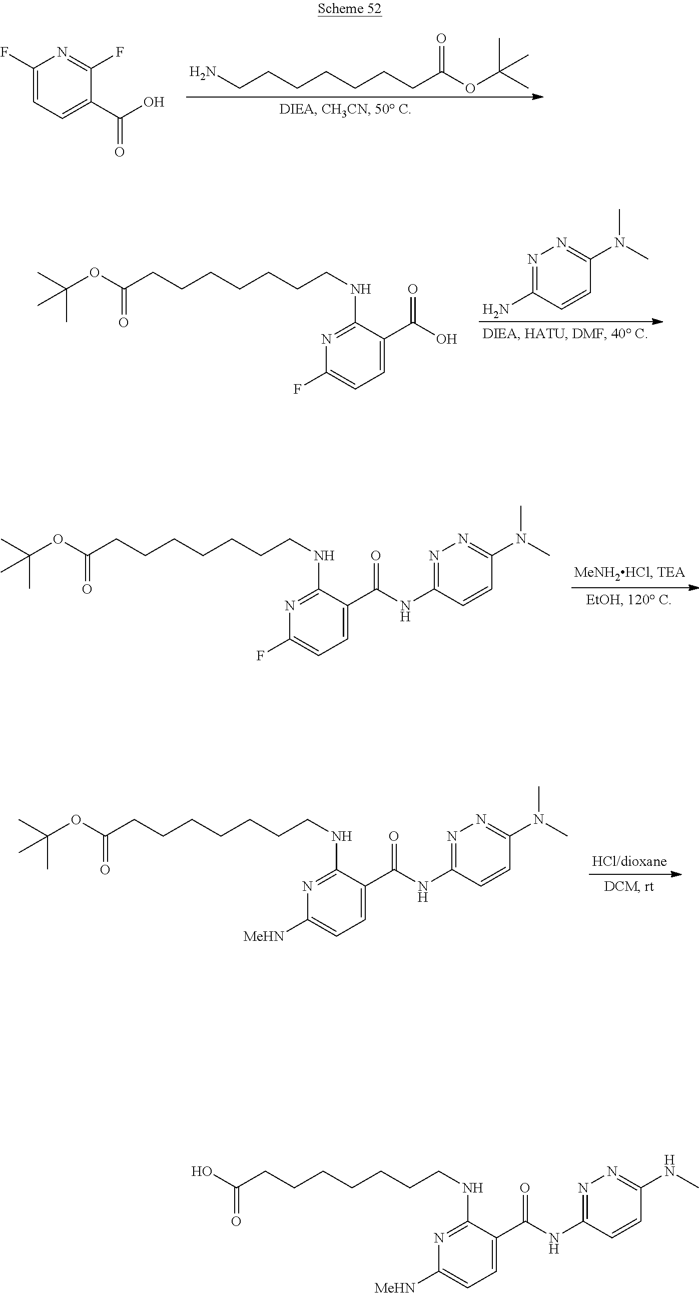

BL1-52 was synthesized following the procedures for preparing BL1-46 (62 mg, 6% yield over 4 steps) as a yellow solid. ¹HNMR (400 MHz, MeOH-d₄) δ 12.22-11.75 (m, 1H), 10.04 (s, 1H), 8.72 (s, 1H), 7.95 (d, J=8.8 Hz, 1H), 7.80 (d, J=10.0 Hz, 1H), 7.13 (d, J=9.6 Hz, 1H), 7.01-6.78 (m, 1H), 5.67 (d, J=8.8 Hz, 1H), 3.44-3.36 (m, 3H), 3.07 (s, 6H), 2.80 (d, J=4.4 Hz, 3H), 2.18 (t, J=7.2 Hz, 3H), 1.55-1.47 (m, 4H), 1.32-1.27 (m, 6H). MS (ESI) m/z=430.5 [M+H]⁺.

### Example 53. 3-(2-((3-((6-(dimethylamino)pyridazin-3-yl)carbamoyl)-6-(methylamino)pyridin-2-yl)amino)ethoxy)propanoic acid (BL1-53)

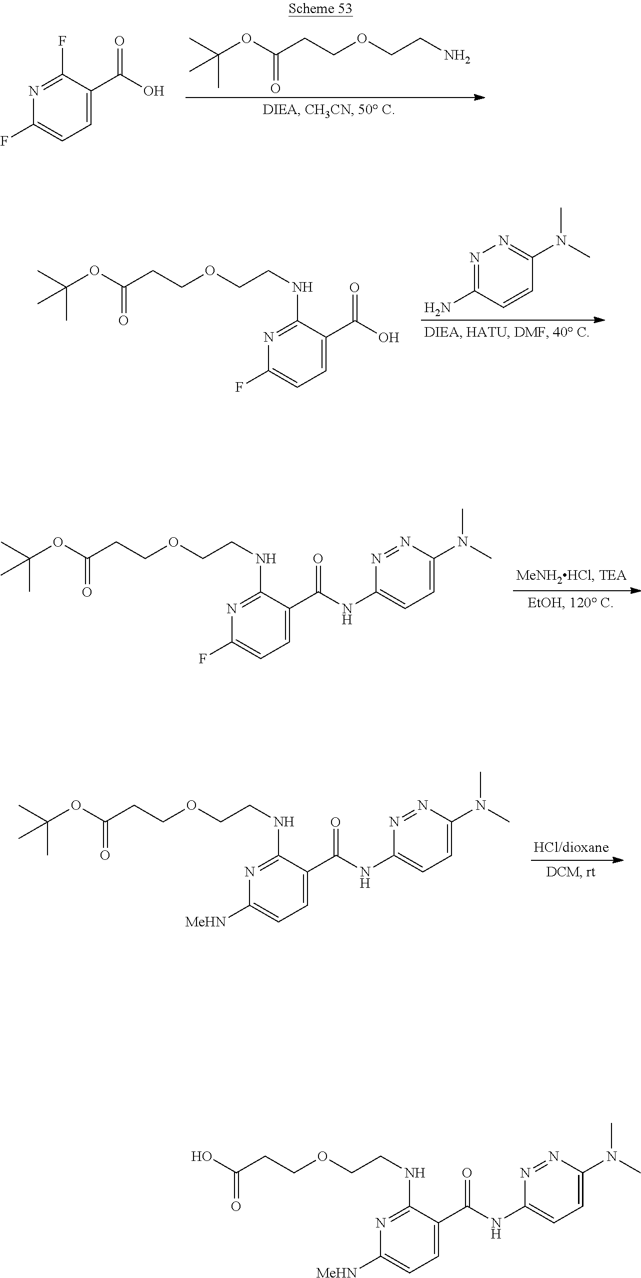

BL1-53 was synthesized following the procedures for preparing BL1-46 (284 mg, 19% yield over 4 steps) as a yellow solid. ¹HNMR (400 MHz, MeOH-d₄) δ 8.40 (d, J=10.4 Hz, 1H), 8.32 (d, J=9.2 Hz, 1H), 7.88 (d, J=10.4 Hz, 1H), 6.15 (d, J=9.2 Hz, 1H), 3.83-3.73 (m, 6H), 3.34 (s, 6H), 3.06 (s, 3H), 2.58 (t, J=5.6 Hz, 2H). MS (ESI) m/z=404.2 [M+H]⁺.

### Example 54. 3-(2-(2-((34(6-(dimethylamino)pyridazin-3-yl)carbamoyl)-6-(methylamino)pyridin-2-yl)amino)ethoxy)ethoxy)propanoic acid (BL1-54)

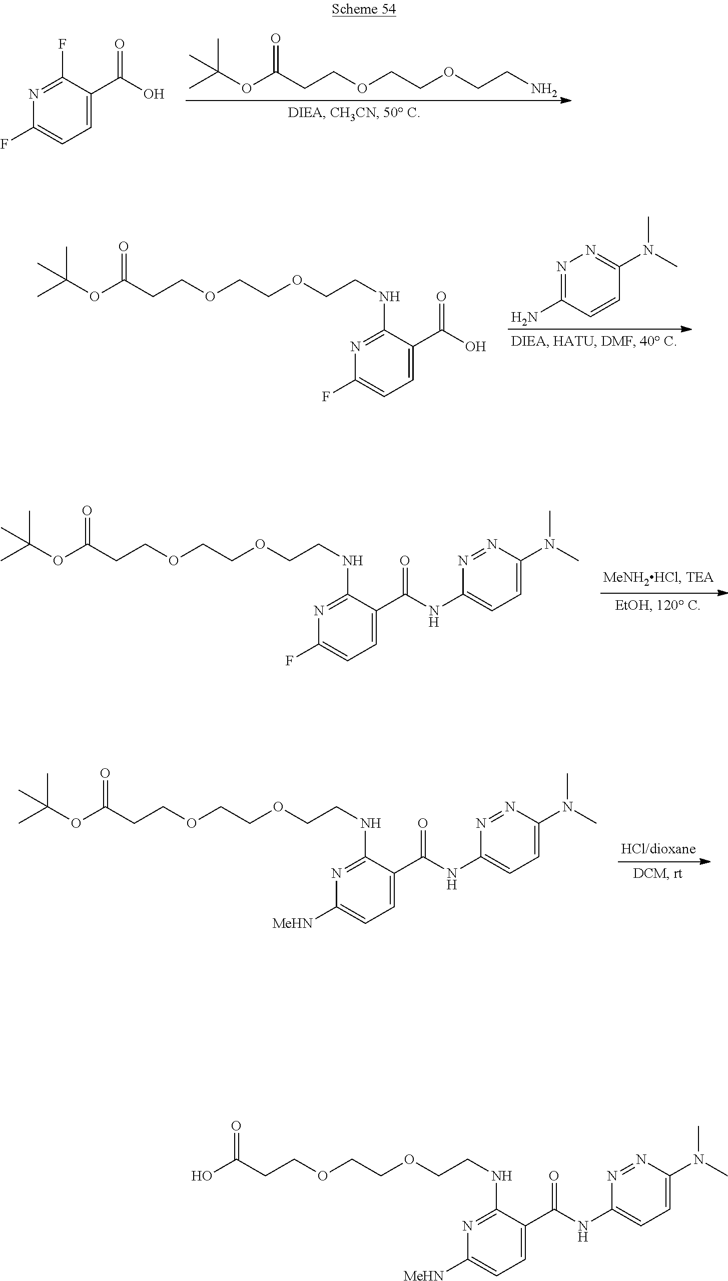

BL1-54 was synthesized following the procedures for preparing BL1-46 (134 mg, 7% yield over 4 steps) as a yellow gum. ¹HNMR (400 MHz, MeOH-d₄) δ 8.37 (d, J=10.0 Hz, 11H), 8.27 (s, 11H), 7.87 (d, J=9.6 Hz, 1H), 6.14 (d, J=9.2 Hz, 1H), 3.81-3.63 (m, 10H), 3.33 (s, 6H), 3.06 (s, 3H), 2.50 (t, J=6.0 Hz, 2H). MS (ESI) m/z=448.2 [M+H]⁺.

### Example 55. tert-butyl 3-(2-(2-(2-((3-((6-(dimethylamino)pyridazin-3-yl)carbamoyl)-6-(methylamino)pyridin-2-yl)amino)ethoxy)ethoxy)ethoxy)propanoate (BL1-55)

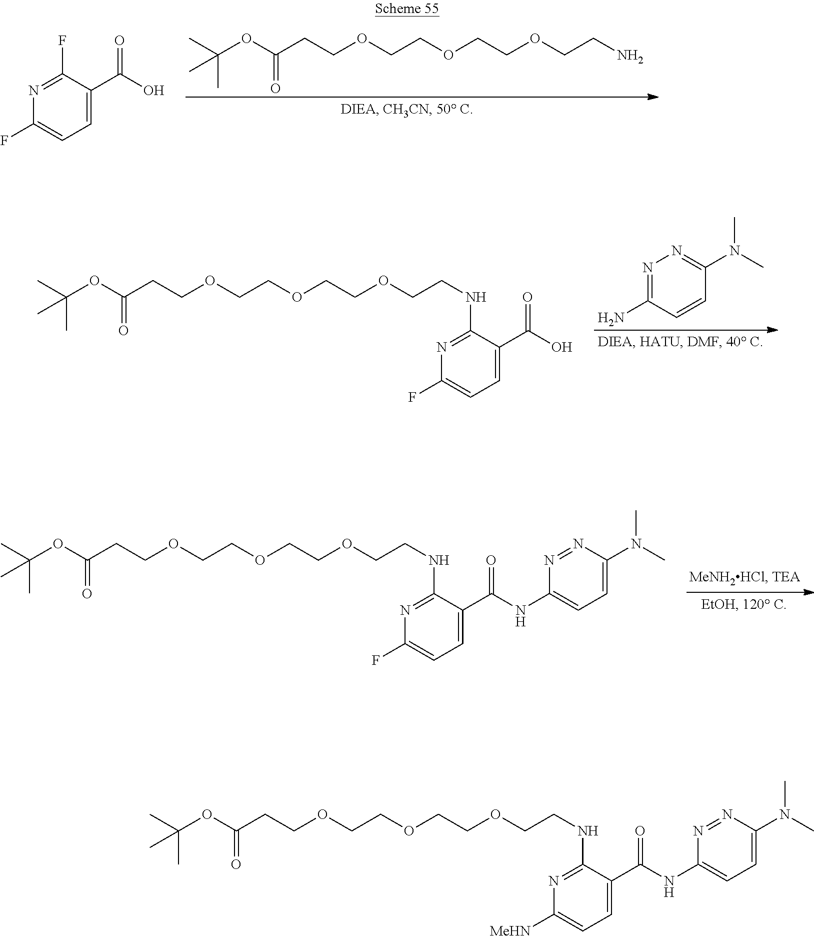

BL1-55 was synthesized following the procedures for preparing BL1-46 (538 mg, 13% yield over 3 steps) as a brown solid. ¹HNMR (400 MHz, MeOH-d₄) δ 8.01 (d, J=10.0 Hz, 1H), 7.78 (d, J=8.8 Hz, 11H), 7.19 (d, J=9.6 Hz, 1H), 5.76 (d, J=8.8 Hz, 1H), 3.69 (s, 4H), 3.67-3.64 (m, 6H), 3.63-3.61 (m, 2H), 3.57-3.54 (m, 2H), 3.13 (s, 6H), 2.91 (s, 3H), 2.44 (t, J=6.0 Hz, 2H), 1.43 (s, 9H). MS (ESI) m/z=548.4 [M+H]⁺.

### Example 56. tert-butyl 1-((3-((6-(dimethylamino)pyridazin-3-yl)carbamoyl)-6-(methylamino)pyridin-2-yl)amino)-3,6,9,12-tetraoxapentadecan-15-oate (BL1-56)

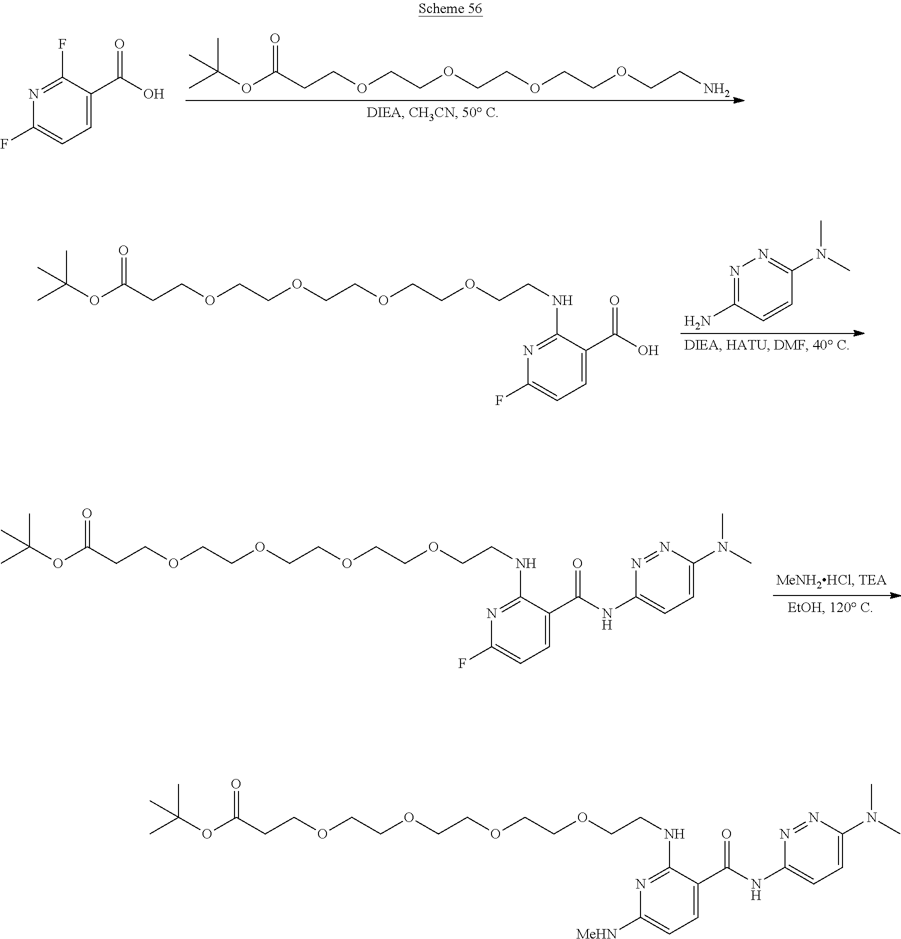

BL1-56 was synthesized following the procedures for preparing BL1-46 (411 mg, 10% yield over 3 steps) as a yellow gum. ¹HNMR (400 MHz, MeOH-d₄) b 8.01 (d, J=10.0 Hz, 1H), 7.78 (d, J=8.8 Hz, 1H), 7.19 (d, J=9.6 Hz, 1H), 5.77 (d, J=8.8 Hz, 1H), 3.70 (s, 4H), 3.67-3.64 (m, 6H), 3.64-3.62 (m, 2H), 3.60-3.58 (m, 2H), 3.58-3.54 (m, 4H), 3.14 (s, 6H), 2.91 (s, 3H), 2.45 (t, J=6.0 Hz, 2H), 1.43 (s, 9H). MS (ESI) m/z=592.4 [M+H]⁺.

### Example 57. 2-((3-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)propyl)amino)-N-(4,5-dimethylthiazol-2-yl)-6-methylnicotinamide (CPD-1)

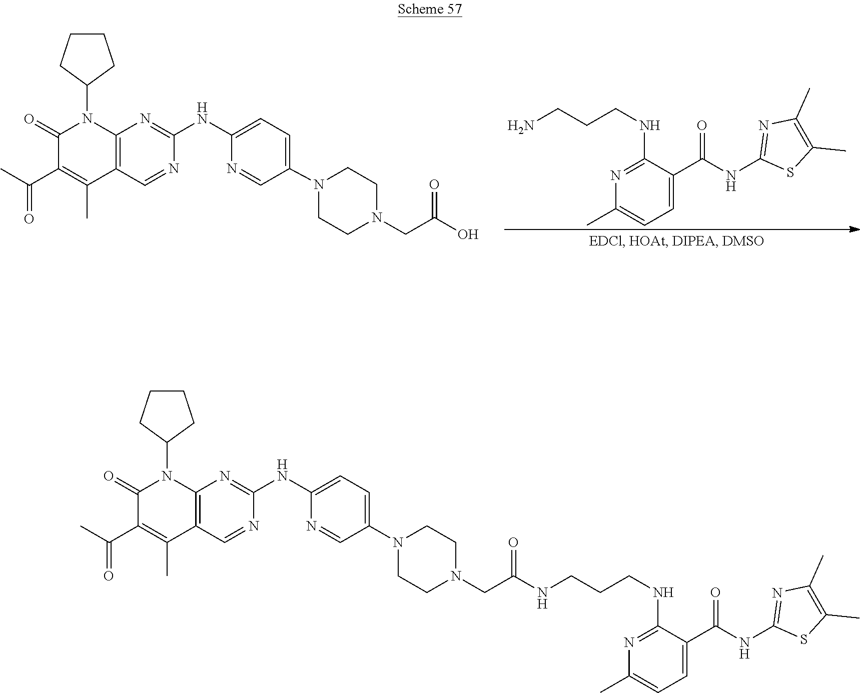 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2nc(C)c(C)s2)c(NCCCN)n1 ; CC(=O)c1c(C)c2cnc(Nc3ccc(N4CCN(CC(=O)NCCCNc5nc(C)ccc5C(=O)Nc5nc(C)c(C)s5)CC4)cn3)nc2n(C2CCCC2)c1=O]

A mixture of 2-(3-aminopropylamino)-N-(4,5-dimethylthiazol-2-yl)-6-methyl-pyridine-3-carboxamide (18 mg, 0.04 mmol), 2-[4-[6-[(6-acetyl-8-cyclopentyl-5-methyl-7-oxo-pyrido[2,3-d]pyrimidin-2-yl)amino]-3-pyridyl]piperazin-1-yl]acetic acid (21.0 mg, 0.04 mmol), HOAt (8.5 mg, 0.06 mmol), EDCl (11.9 mg, 0.06 mmol) and DIPEA (26.8 mg, 0.21 mmol) in DMSO (2 mL) was stirred at rt for 16 h. The reaction mixture was poured into water (20 mL) and extracted with ethyl acetate (3×20 mL). The combined organic layers were washed with saturated brine (20 mL), dried over anhydrous sodium sulfate, filtered, and evaporated under reduced pressure. The resulting residue was purified by reverse-phase chromatography and pre-TLC to provide the desired product (9.8 mg, 29% yield) as a yellow solid. MS (ESI) m/z=807.4 [M+H]⁺.

### Example 58. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-methoxypyridazin-3-yl)-6-methylnicotinamide (CPD-2)

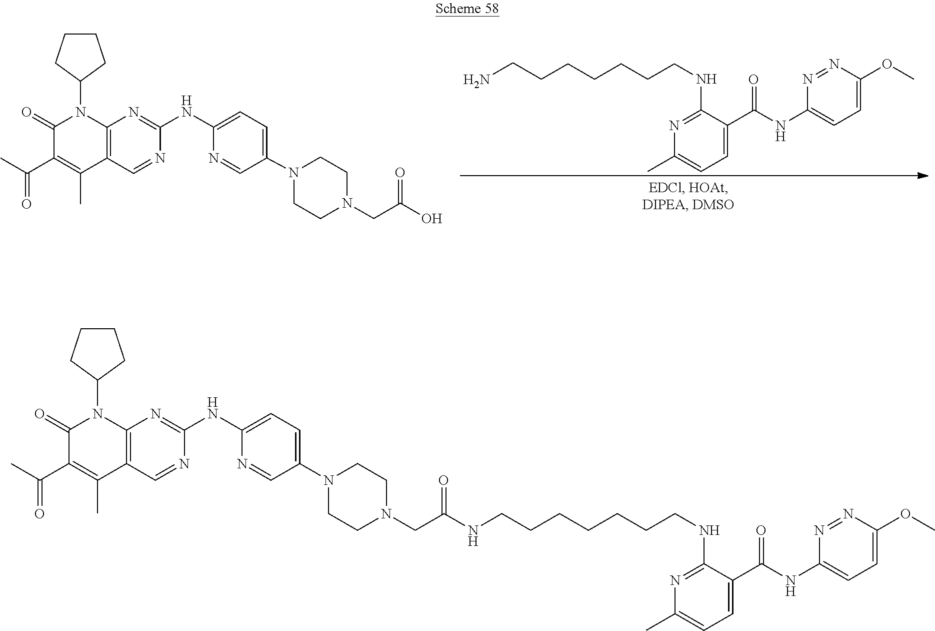 [SMILES: CC(=O)c1c(C)c2cnc(Nc3ccc(N4CCN(CC(=O)O)CC4)cn3)nc2n(C2CCCC2)c1=O ; COc1ccc(NC(=O)c2ccc(C)nc2NCCCCCCCN)nn1 ; COc1ccc(NC(=O)c2ccc(C)nc2NCCCCCCCNC(=O)CN2CCN(c3ccc(Nc4ncc5c(C)c(C(C)=O)c(=O)n(C6CCCC6)c5n4)nc3)CC2)nn1]

CPD-2 was synthesized following the standard procedure for preparing CPD-1 (8.3 mg, 24% yield) as a yellow solid. MS (ESI) m/z=860.4 [M+H]⁺.

### Example 59. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4,5-dimethylthiazol-2-yl)-6-methylnicotinamide (CPD-3)

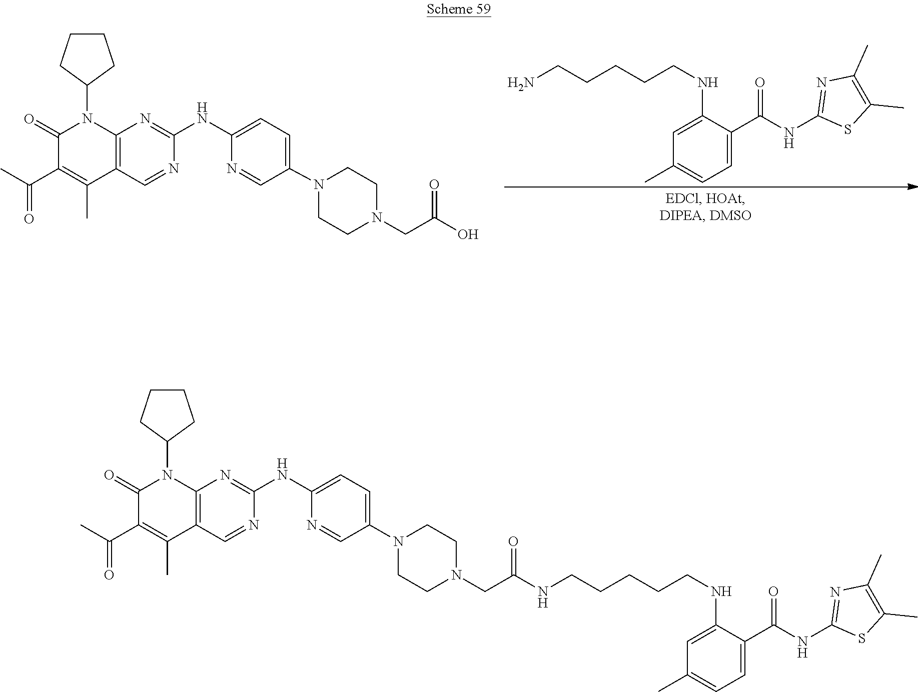 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2nc(C)c(C)s2)c(NCCCCCN)c1 ; CC(=O)c1c(C)c2cnc(Nc3ccc(N4CCN(CC(=O)NCCCCCNc5cc(C)ccc5C(=O)Nc5nc(C)c(C)s5)CC4)cn3)nc2n(C2CCCC2)c1=O]

CPD-3 was synthesized following the standard procedure for preparing CPD-1 (4.7 mg, 16% yield) as a yellow solid. MS (ESI) m/z=835.5 [M+H]⁺.

### Example 60. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-6-(methylamino)nicotinamide (CPD-4)

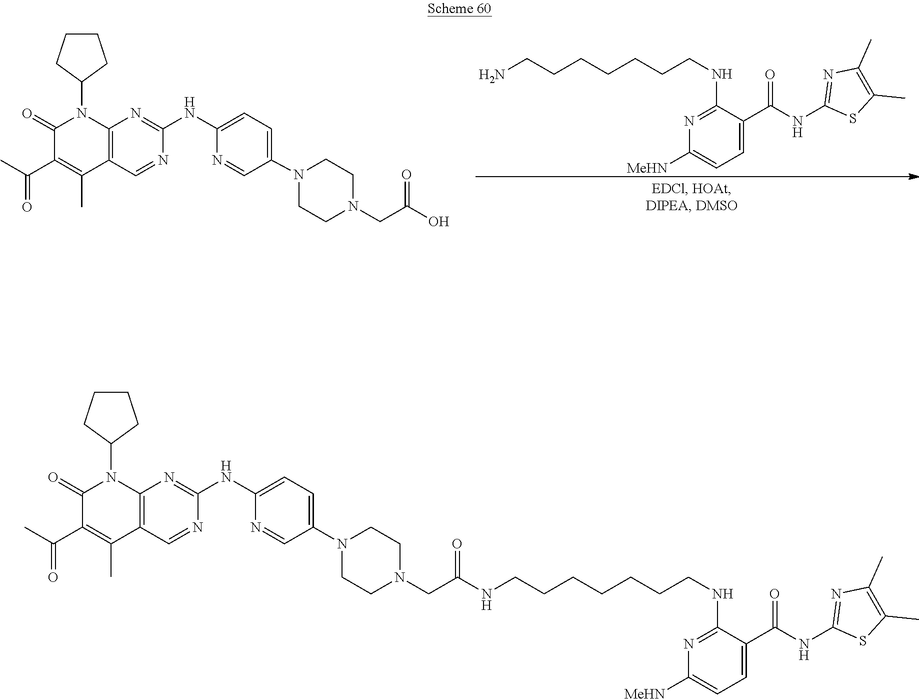 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2nc(C)c(C)s2)c(NCCCCCCCN)n1 ; CNc1ccc(C(=O)Nc2nc(C)c(C)s2)c(NCCCCCCCNC(=O)CN2CCN(c3ccc(Nc4ncc5c(C)c(C(C)=O)c(=O)n(C6CCCC6)c5n4)nc3)CC2)n1]

CPD-4 was synthesized following the standard procedure for preparing CPD-1 (5.8 mg, 22% yield) as a yellow solid. MS (ESI) m/z=878.5 [M+H]⁺.

### Example 61. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(methylamino)nicotinamide (CPD-5)

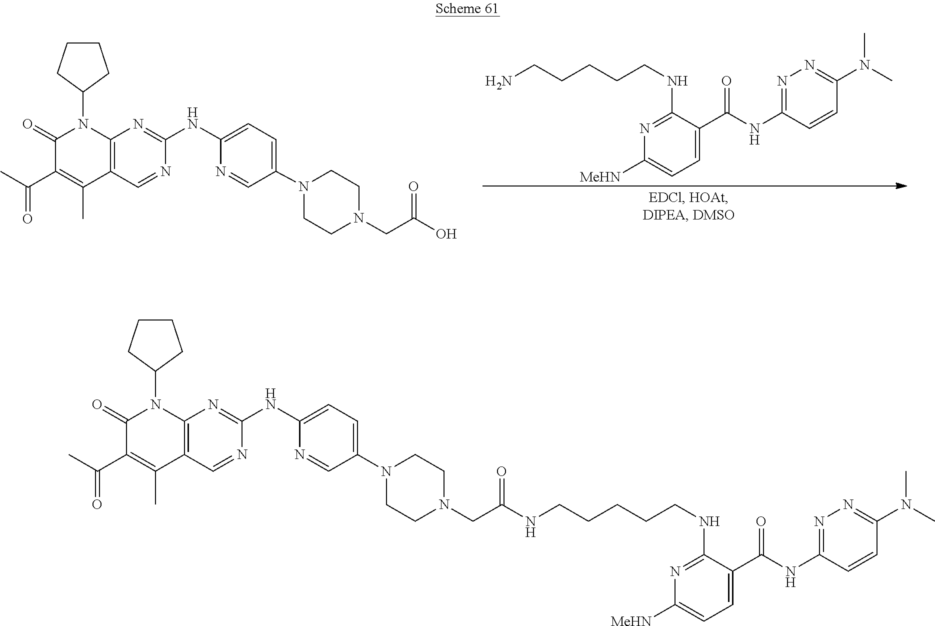 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(NCCCCCN)n1 ; CNc1ccc(C(=O)Nc2ccc(N(C)C)nn2)c(NCCCCCNC(=O)CN2CCN(c3ccc(Nc4ncc5c(C)c(C(C)=O)c(=O)n(C6CCCC6)c5n4)nc3)CC2)n1]

CPD-5 was synthesized following the standard procedure for preparing CPD-1 (5.1 mg, 19% yield) as a yellow solid. MS (ESI) m/z=860.5 [M+H]⁺.

### Example 62. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-cyclopropyl-5-methylpyridin-2-yl)-6-(methylamino)nicotinamide (CPD-6)

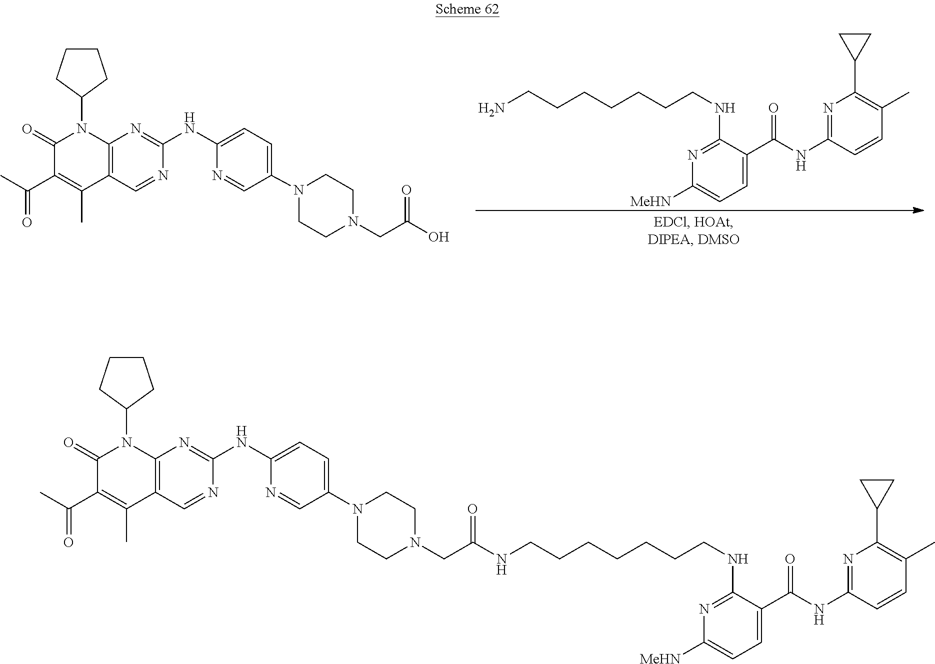 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(C)c(C3CC3)n2)c(NCCCCCCCN)n1 ; CNc1ccc(C(=O)Nc2ccc(C)c(C3CC3)n2)c(NCCCCCCCNC(=O)CN2CCN(c3ccc(Nc4ncc5c(C)c(C(C)=O)c(=O)n(C6CCCC6)c5n4)nc3)CC2)n1]

CPD-6 was synthesized following the standard procedure for preparing CPD-1 (7.8 mg, 30% yield) as a yellow solid. MS (ESI) m/z=898.6 [M+H]⁺.

### Example 63. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-5-methylpyridin-2-yl)-6-(methylamino)nicotinamide (CPD-7)

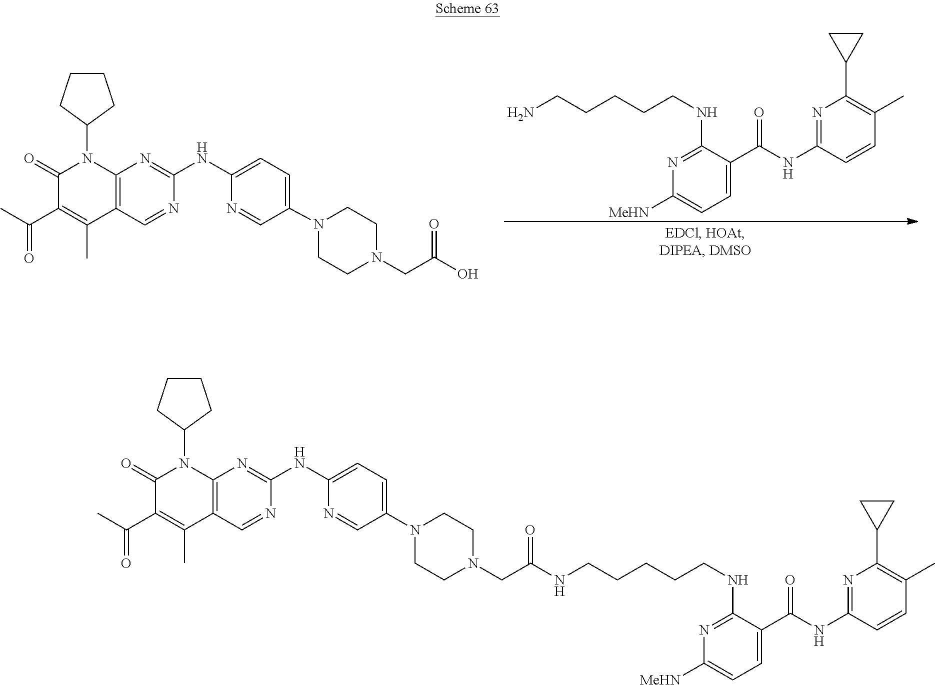 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(C)c(C3CC3)n2)c(NCCCCCN)n1 ; CNc1ccc(C(=O)Nc2ccc(C)c(C3CC3)n2)c(NCCCCCNC(=O)CN2CCN(c3ccc(Nc4ncc5c(C)c(C(C)=O)c(=O)n(C6CCCC6)c5n4)nc3)CC2)n1]

CPD-7 was synthesized following the standard procedure for preparing CPD-1 (16.6 mg, 63% yield) as a yellow solid. MS (ESI) m/z=870.5 [M+H]⁺.

### Example 64. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-(dimethylamino)pyridazin-3-yl)-6-methylnicotinamide (CPD-8)

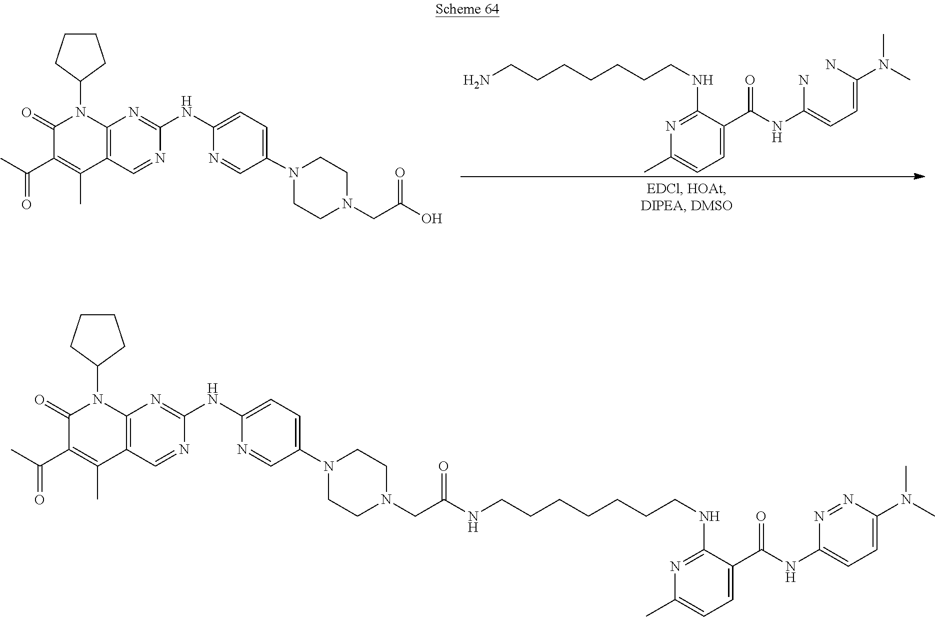 [SMILES: CC(=O)c1c(C)c2cnc(Nc3ccc(N4CCN(CC(=O)O)CC4)cn3)nc2n(C2CCCC2)c1=O ; Cc1ccc(C(=O)N/C(N)=C/C=C(\N)N(C)C)c(NCCCCCCCN)n1 ; CC(=O)c1c(C)c2cnc(Nc3ccc(N4CCN(CC(=O)NCCCCCCCNc5nc(C)ccc5C(=O)Nc5ccc(N(C)C)nn5)CC4)cn3)nc2n(C2CCCC2)c1=O]

CPD-8 was synthesized following the standard procedure for preparing CPD-1 (9.3 mg, 38% yield) as a yellow solid. MS (ESI) m/z=873.4 [M+H]⁺.

### Example 65. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-cyclopropyl-5-methylpyridin-2-yl)-6-methylnicotinamide (CPD-9)

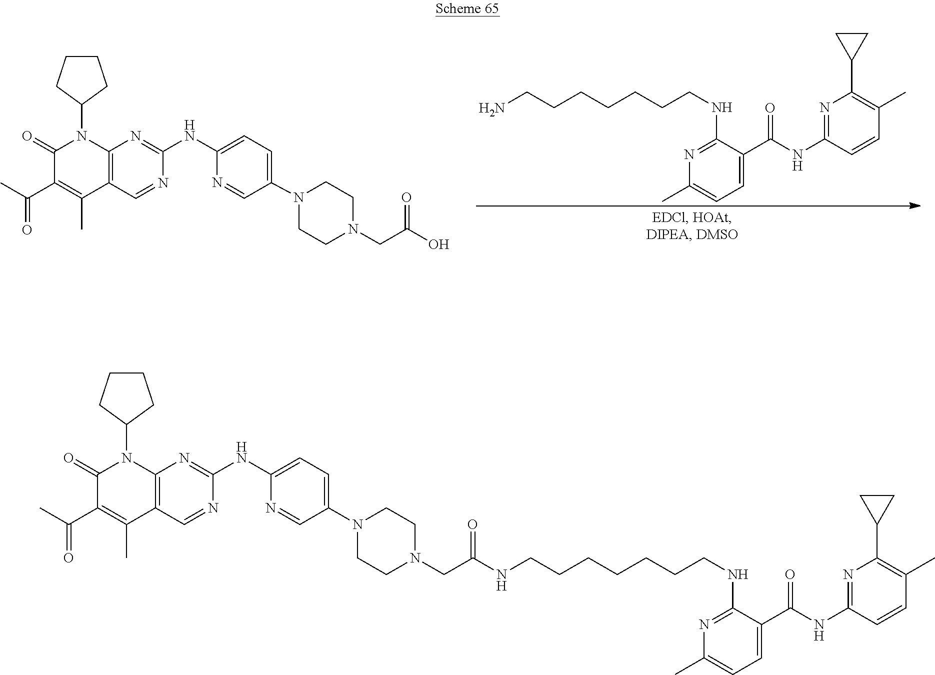 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2ccc(C)c(C3CC3)n2)c(NCCCCCCCN)n1 ; CC(=O)c1c(C)c2cnc(Nc3ccc(N4CCN(CC(=O)NCCCCCCCNc5nc(C)ccc5C(=O)Nc5ccc(C)c(C6CC6)n5)CC4)cn3)nc2n(C2CCCC2)c1=O]

CPD-9 was synthesized following the standard procedure for preparing CPD-1 (7.9 mg, 45% yield) as a yellow solid. MS (ESI) m/z=883.7 [M+H]⁺.

### Example 66. 2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-(dimethylamino)pyridazin-3-yl)-6-(methylamino)nicotinamide (CPD-10)

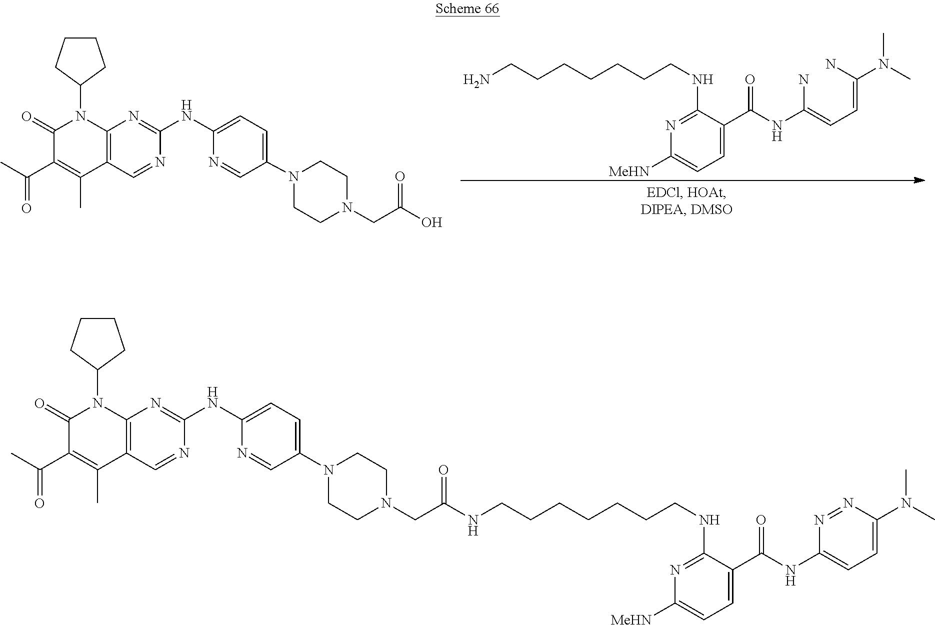 [SMILES: CC(=O)c1c(C)c2cnc(Nc3ccc(N4CCN(CC(=O)O)CC4)cn3)nc2n(C2CCCC2)c1=O ; CNc1ccc(C(=O)N/C(N)=C/C=C(\N)N(C)C)c(NCCCCCCCN)n1 ; CNc1ccc(C(=O)Nc2ccc(N(C)C)nn2)c(NCCCCCCCNC(=O)CN2CCN(c3ccc(Nc4ncc5c(C)c(C(C)=O)c(=O)n(C6CCCC6)c5n4)nc3)CC2)n1]

CPD-10 was synthesized following the standard procedure for preparing CPD-1 (41.3 mg, 39% yield) as a yellow solid. MS (ESI) m/z=888.5 [M+H]⁺.

### Example 67. 2-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(6-methoxypyridazin-3-yl)-6-(methylamino)nicotinamide (CPD-11)

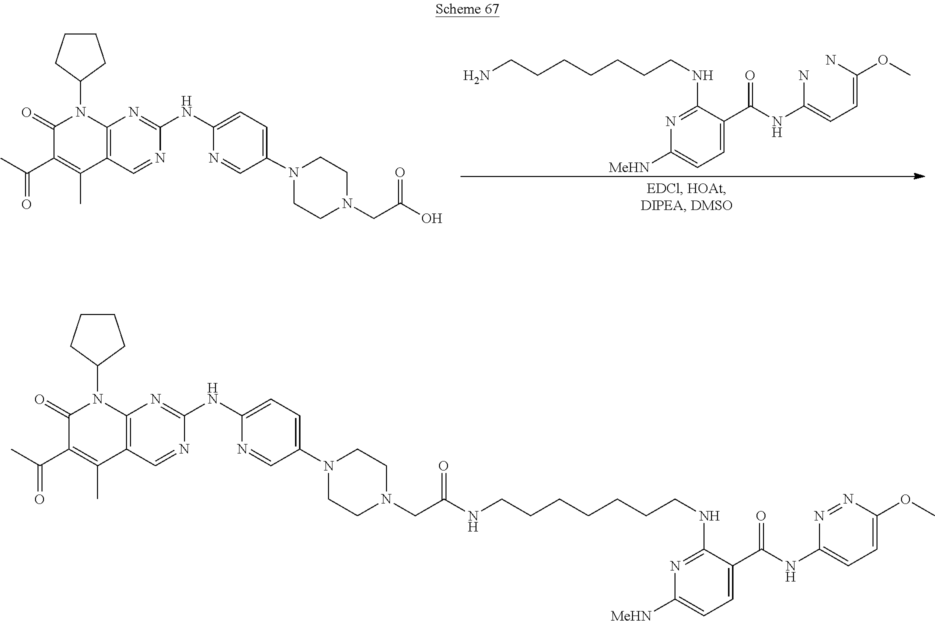 [SMILES: CC(=O)c1c(C)c2cnc(Nc3ccc(N4CCN(CC(=O)O)CC4)cn3)nc2n(C2CCCC2)c1=O ; CNc1ccc(C(=O)N/C(N)=C/C=C(\N)OC)c(NCCCCCCCN)n1 ; CNc1ccc(C(=O)Nc2ccc(OC)nn2)c(NCCCCCCCNC(=O)CN2CCN(c3ccc(Nc4ncc5c(C)c(C(C)=O)c(=O)n(C6CCCC6)c5n4)nc3)CC2)n1]

CPD-11 was synthesized following the standard procedure for preparing CPD-1 (9.6 mg, 45% yield) as a yellow solid. MS (ESI) m/z=875.6 [M+H]⁺.

### Example 68. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propanamido)pentyl)amino)-N-(6-(dimethylamino)pyridazin-3-yl)-6-(methylamino)nicotinamide (CPD-14)

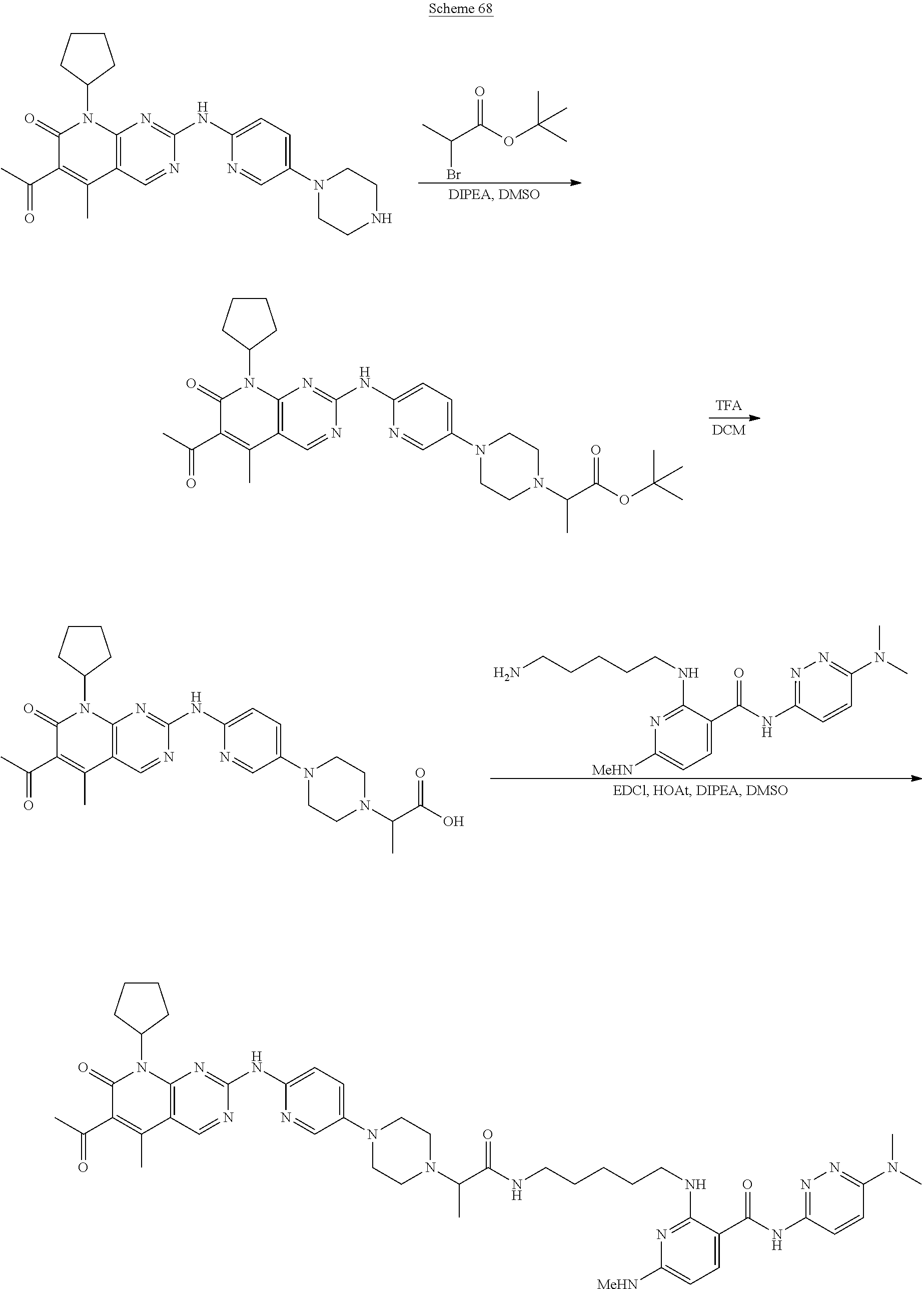

### Step 1. Synthesis of tert-butyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoate

To a mixture of 6-acetyl-8-cyclopentyl-5-methyl-2-((5-(piperazin-1-yl)pyridin-2-yl)amino)pyrido[2,3-d]pyrimidin-7(8H)-one (30 mg, 0.06 mmol) and DIPEA (23 mg, 0.18 mmol) in DMSO (2 mL) was added tert-butyl 2-bromopropanoate (13 mg, 0.06 mmol) at 0 [symbol] . The reaction mixture was stirred at 100° C. for 16 h. After cooling down to rt, the reaction mixture was quenched with water (20 mL) and extracted with ethyl acetate (20 mL×2). The combined organic layers were washed with saturated brine (20 mL), dried over anhydrous sodium sulfate, filtered, and evaporated under reduced pressure to provide the desired product (25 mg, 64%) as a colorless oil. MS (ESI) m/z=576.5 [M+H]⁺.

### Step 2. Synthesis of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoic acid

To a solution of tert-butyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoate (25 mg, 0.04 mmol) in DCM (2 mL) was added TFA (1 mL) at 0 [symbol] . The reaction mixture was stirred at rt for 1 h. The solution was concentrated under reduced pressure to provide the desired product (18 mg, 70% yield) as a yellow oil, which was used in the next step directly without further purification. MS (ESI) m/z=520.5 [M+H]⁺.

### Step 3. Synthesis of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)pentyl)amino)-N-(6-(dimethylamino)pyridazin-3-yl)-6-(methylamino)nicotinamide

The title compound was synthesized following the standard procedure for preparing CPD-1 (1.1 mg, 13% yield) as a yellow solid. MS (ESI) m/z=876.0 [M+H]⁺.

### Example 69. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-isopropylpyridazin-3-yl)-6-(methylamino)nicotinamide (CPD-15)

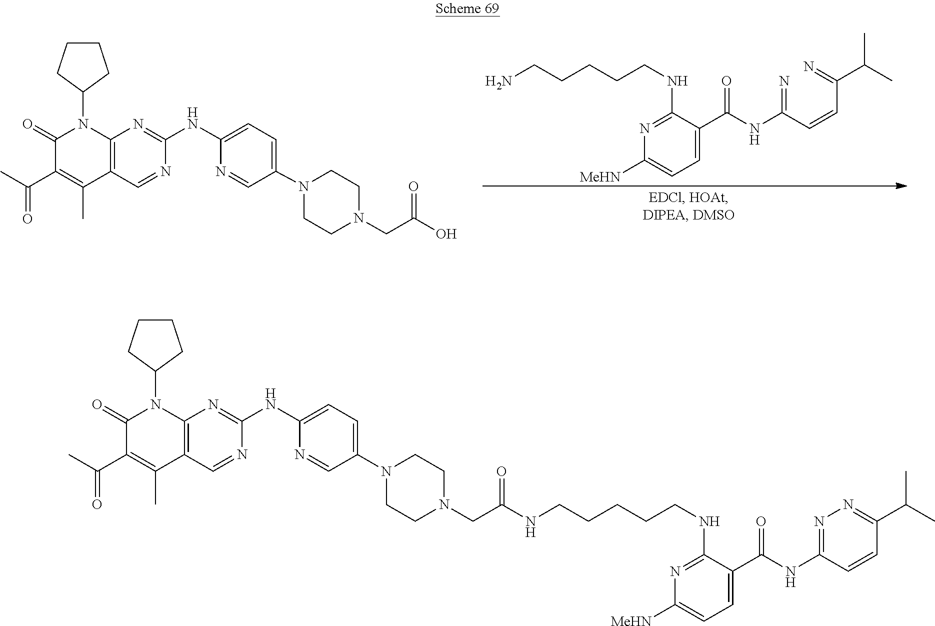 [SMILES: CC(=O)c1c(C)c2cnc(Nc3ccc(N4CCN(CC(=O)O)CC4)cn3)nc2n(C2CCCC2)c1=O ; CNc1ccc(C(=O)NC(=N)/C=C\C(=N)C(C)C)c(NCCCCCN)n1 ; CNc1ccc(C(=O)Nc2ccc(C(C)C)nn2)c(NCCCCCNC(=O)CN2CCN(c3ccc(Nc4ncc5c(C)c(C(C)=O)c(=O)n(C6CCCC6)c5n4)nc3)CC2)n1]

CPD-15 was synthesized following the standard procedure for preparing CPD-1 (11 mg, 48% yield) as a yellow solid. MS (ESI) nm/z=859.7 [M+H]⁺.

### Example 70. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-methylnicotinamide (CPD-16)

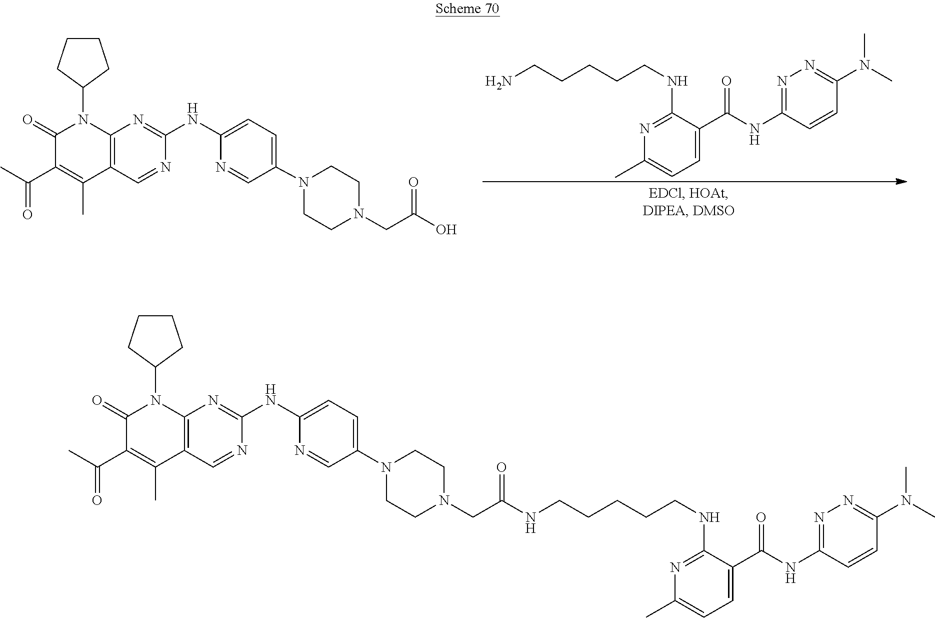 [SMILES: CC(=O)c1c(C)c2cnc(Nc3ccc(N4CCN(CC(=O)O)CC4)cn3)nc2n(C2CCCC2)c1=O ; Cc1ccc(C(=O)Nc2ccc(N(C)C)nn2)c(NCCCCCN)n1 ; CC(=O)c1c(C)c2cnc(Nc3ccc(N4CCN(CC(=O)NCCCCCNc5nc(C)ccc5C(=O)Nc5ccc(N(C)C)nn5)CC4)cn3)nc2n(C2CCCC2)c1=O]

CPD-16 was synthesized following the standard procedure for preparing CPD-1 (2.7 mg, 12% yield) as a yellow solid. MS (ESI) m/z=845.7 [M+H]⁺.

### Example 71. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(pyrrolidin-1-yl)pyridazin-3-yl)nicotinamide (CPD-17)

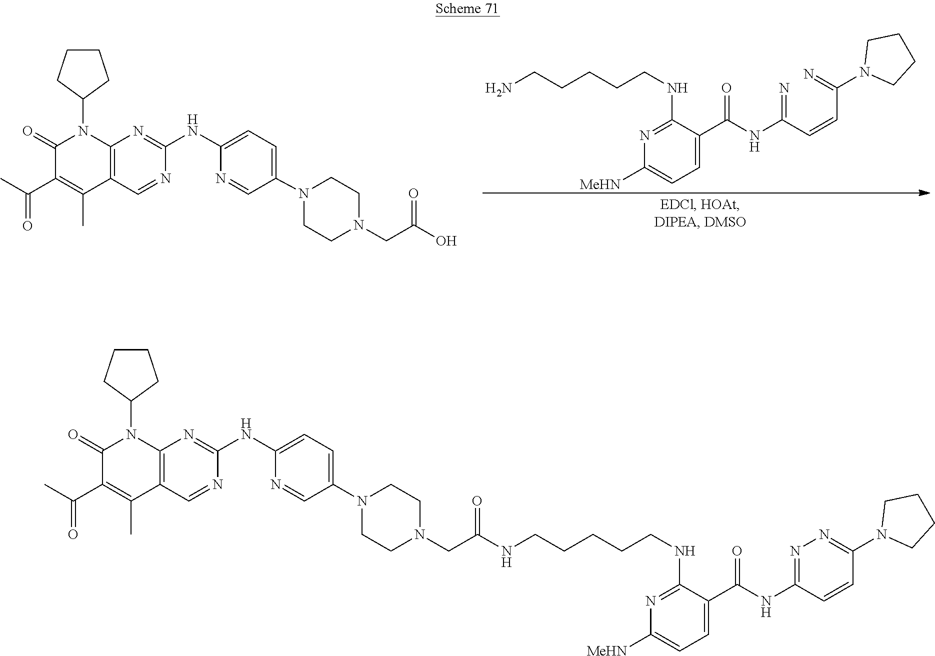 [SMILES: CC(=O)c1c(C)c2cnc(Nc3ccc(N4CCN(CC(=O)O)CC4)cn3)nc2n(C2CCCC2)c1=O ; CNc1ccc(C(=O)NC(=N)/C=C\C(=N)N2CCCC2)c(NCCCCCN)n1 ; CNc1ccc(C(=O)Nc2ccc(N3CCCC3)nn2)c(NCCCCCNC(=O)CN2CCN(c3ccc(Nc4ncc5c(C)c(C(C)=O)c(=O)n(C6CCCC6)c5n4)nc3)CC2)n1]

CPD-17 was synthesized following the standard procedure for preparing CPD-1 (8.8 mg, 46% yield) as a yellow solid. MS (ESI) m/z=886.7 [M+H]⁺.

### Example 72. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(isopropylamino)nicotinamide (CPD-18)

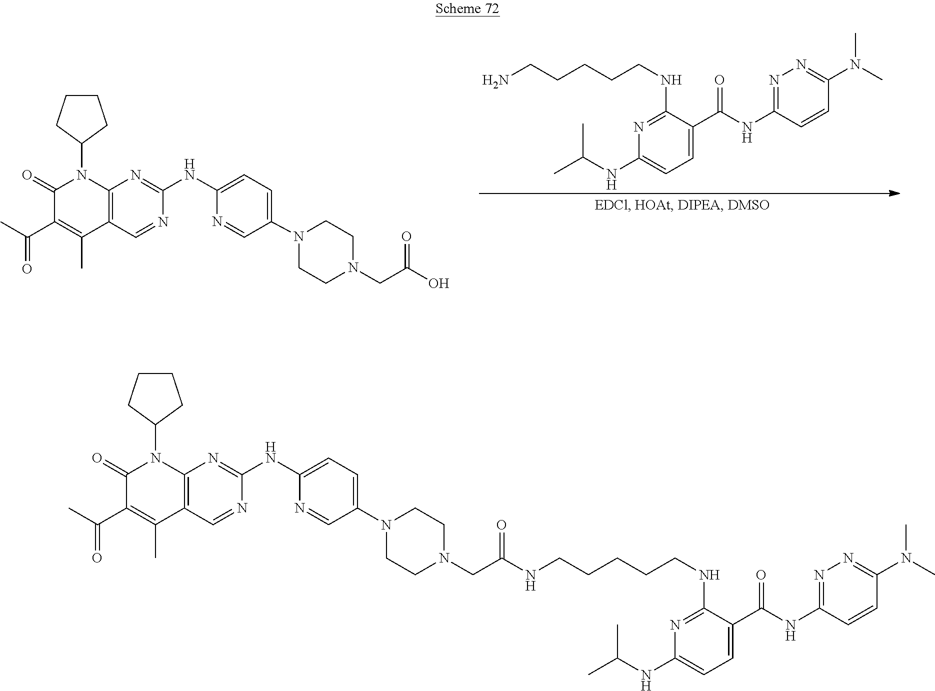 [SMILES: CC(=O)c1c(C)c2cnc(Nc3ccc(N4CCN(CC(=O)O)CC4)cn3)nc2n(C2CCCC2)c1=O ; CC(C)Nc1ccc(C(=O)Nc2ccc(N(C)C)nn2)c(NCCCCCN)n1 ; CC(=O)c1c(C)c2cnc(Nc3ccc(N4CCN(CC(=O)NCCCCCNc5nc(NC(C)C)ccc5C(=O)Nc5ccc(N(C)C)nn5)CC4)cn3)nc2n(C2CCCC2)c1=O]

CPD-18 was synthesized following the standard procedure for preparing CPD-1 (10.2 mg, 35% yield) as a yellow solid. MS (ESI) m/z=888.9 [M+H]⁺.

### Example 73. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(trifluoromethyl)pyridazin-3-yl)nicotinamide (CPD-19)

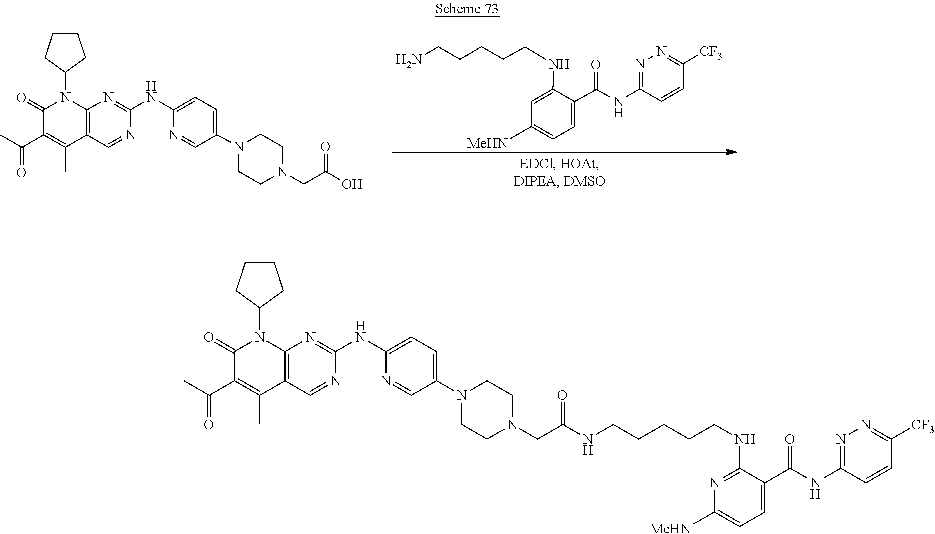 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(C(F)(F)F)nn2)c(NCCCCCN)c1 ; CNc1ccc(C(=O)Nc2ccc(C(F)(F)F)nn2)c(NCCCCCNC(=O)CN2CCN(c3ccc(Nc4ncc5c(C)c(C(C)=O)c(=O)n(C6CCCC6)c5n4)nc3)CC2)n1]

CPD-19 was synthesized following the standard procedure for preparing CPD-1 (10 mg, 34% yield) as a yellow solid. MS (ESI) n/z=885.7 [M+H]⁺.

### Example 74. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-chloropyridin-2-yl)-6-(methylamino)nicotinamide (CPD-20)

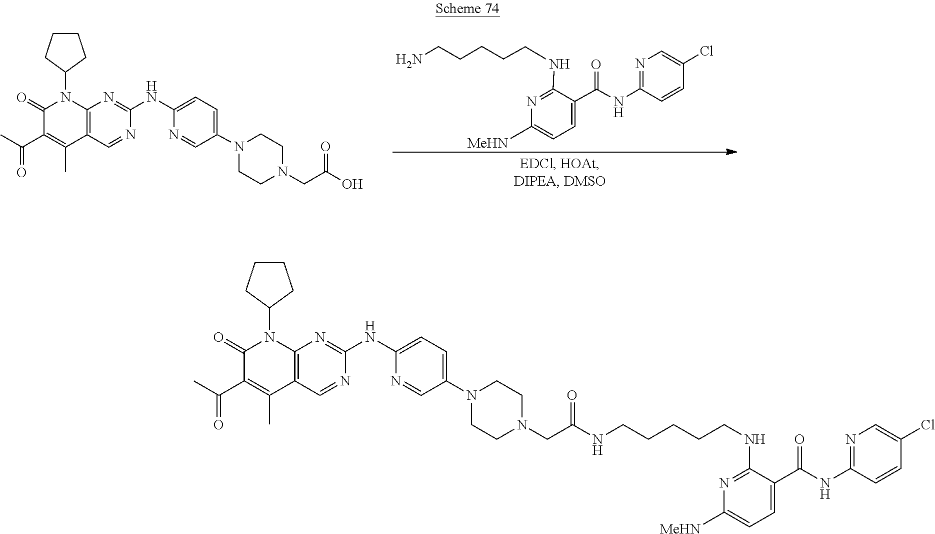 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(Cl)cn2)c(NCCCCCN)n1 ; CNc1ccc(C(=O)Nc2ccc(Cl)cn2)c(NCCCCCNC(=O)CN2CCN(c3ccc(Nc4ncc5c(C)c(C(C)=O)c(=O)n(C6CCCC6)c5n4)nc3)CC2)n1]

CPD-20 was synthesized following the standard procedure for preparing CPD-1 (7.5 mg, 25% yield) as a yellow solid. MS (ESI) m/z=850.7 [M+H].

### Example 75. 2-((5-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)pentyl)amino)-N-(6-(dimethylamino)pyridazin-3-yl)-6-(methylamino)nicotinamide (CPD-21)

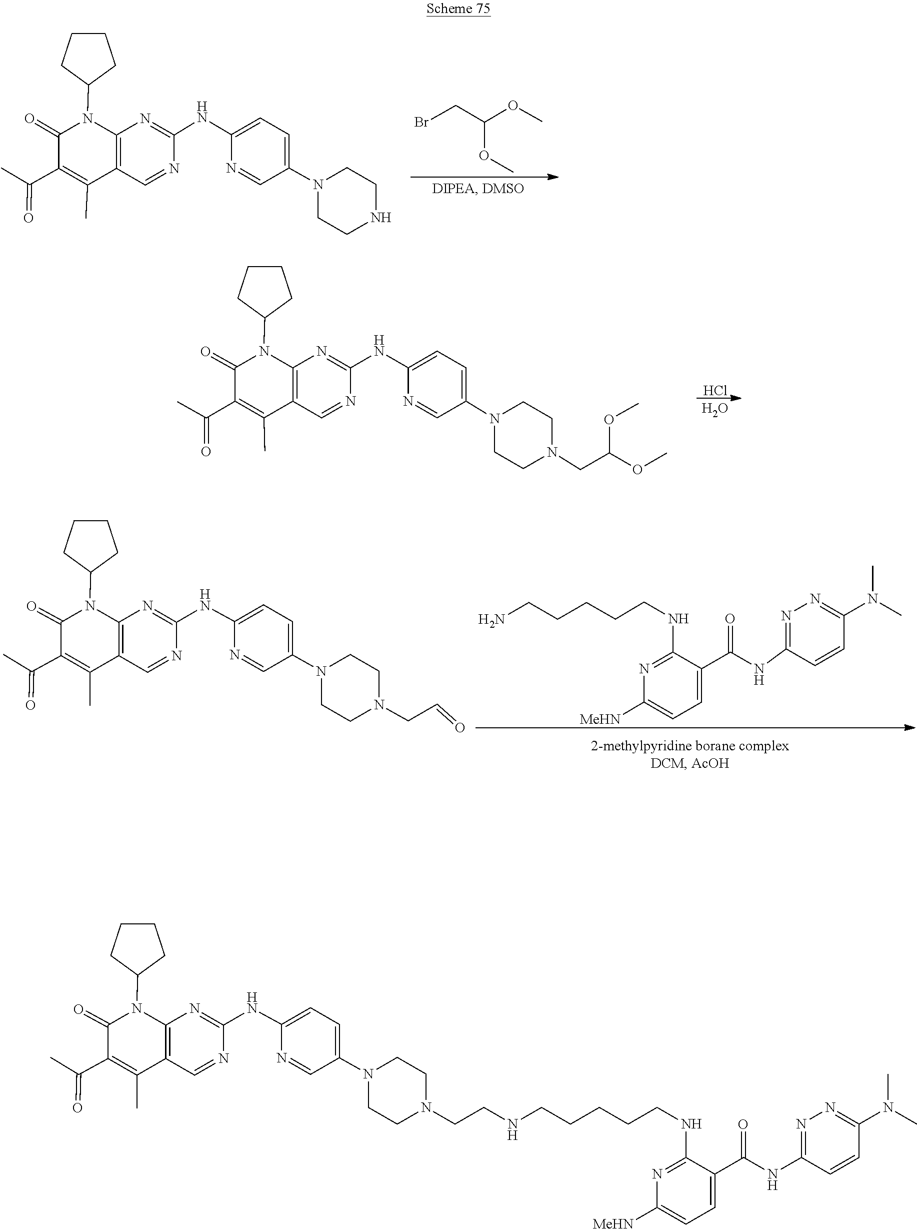

### Step 1. Synthesis of 6-acetyl-8-cyclopentyl-2-((5-(4-(2,2-dimethoxyethyl)piperazin-1-yl)pyridin-2-yl)amino)-5-methylpyrido[2,3-d]pyrimidin-7(8H)-one

To a mixture of 6-acetyl-8-cyclopentyl-5-methyl-2-((5-(piperazin-1-yl)pyridin-2-yl)amino)pyrido[2,3-d]pyrimidin-7(8H)-one (400 mg, 0.89 mmol) and DIPEA (347 mg, 2.67 mmol) in DMSO (5 mL) was added 2-bromo-1,1-dimethoxyethane (150 mg, 0.89 mmol) at rt. The reaction mixture was stirred at 70° C. for 16 h. After cooling down to rt, the reaction was quenched with water (30 mL) and extracted with ethyl acetate (20 mL×2). The combined organic layers were washed with brine (20 mL), dried over anhydrous sodium sulfate, filtered and concentrated to provide the desired product (450 mg, 94%) as a colorless oil. MS (ESI) m/z=536.6 [M+H]⁺.

### Step 2. Synthesis of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetaldehyde

A solution of 6-acetyl-8-cyclopentyl-2-((5-(4-(2,2-dimethoxyethyl)piperazin-1-yl)pyridin-2-yl)amino)-5-methylpyrido[2,3-d]pyrimidin-7(8H)-one (230 mg, 0.4 mmol) in aq. HCl (1 M, 10 mL) was stirred at 50° C. for 16 h. After cooling down to rt, the reaction mixture was extracted with ethyl acetate (20 mL×2). The combined organic layers were washed brine, dried over anhydrous sodium sulfate, filtered, and concentrated to provide the desired product (160 mg, 76%) as a colorless oil. MS (ESI) m/z=490.4 [M+H]⁺.

### Step 3. Synthesis of 2-((5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)pentyl)amino)-N-(6-(dimethylamino)pyridazin-3-yl)-6-(methylamino)nicotinamide

To a mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetaldehyde (60 mg, 0.1 mmol) and 2-((5-aminopentyl)amino)-N-(6-(dimethylamino)pyridazin-3-yl)-6-(methylamino)nicotinamide (30 mg, 0.05 mmol) in DCM (10 mL) and AcOH (1 mL) was added 2-methylpyridine borane complex (13 mg, 0.1 mmol) at 0 [symbol] . Then the reaction mixture was stirred at rt for 16 h. The reaction mixture was poured into water (20 mL) and extracted with ethyl acetate (3×20 mL). The combined organic layers were washed with saturated brine (20 mL), dried over anhydrous sodium sulfate, filtered, and evaporated under reduced pressure. The residue was purified by reverse-phase chromatography to provide the desired product (7.4 mg, 12% yield) as a yellow solid. MS (ESI) m/z=846.9 [M+H]⁺.

### Example 76. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-N-(6-(dimethylamino)pyridazin-3-yl)-6-(methylamino)nicotinamide (CPD-22)

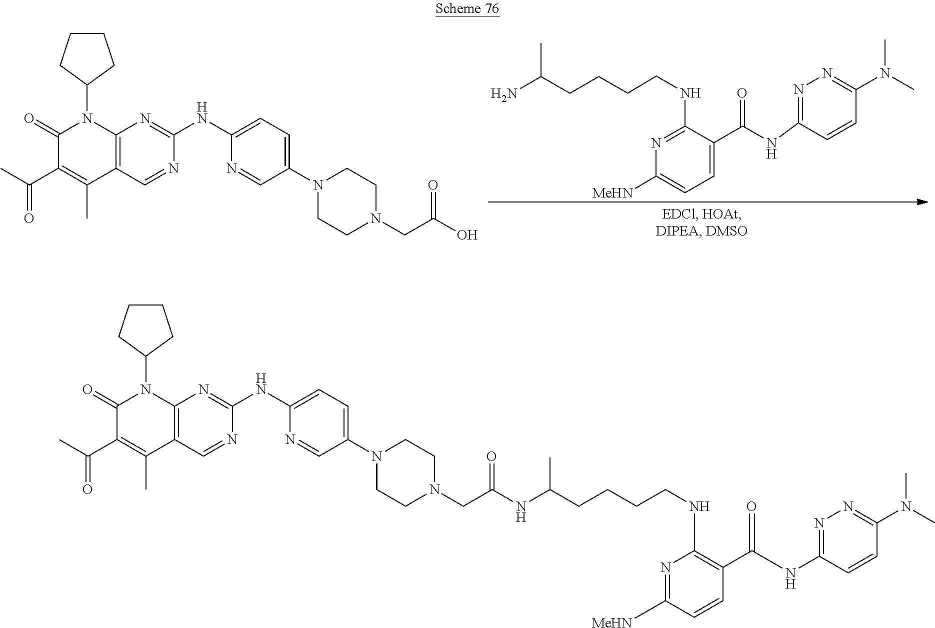 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(NCCCCC(C)N)n1 ; CNc1ccc(C(=O)Nc2ccc(N(C)C)nn2)c(NCCCCC(C)NC(=O)CN2CCN(c3ccc(Nc4ncc5c(C)c(C(C)=O)c(=O)n(C6CCCC6)c5n4)nc3)CC2)n1]

CPD-22 was synthesized following the standard procedure for preparing CPD-1 (12.5 mg, 49% yield) as a yellow solid. MS (ESI) m/z=874.9 [M+H]⁺.

### Example 77. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)(methyl)amino)-N-(6-(dimethylamino)pyridazin-3-yl)-6-(methylamino)nicotinamide (CPD-23)

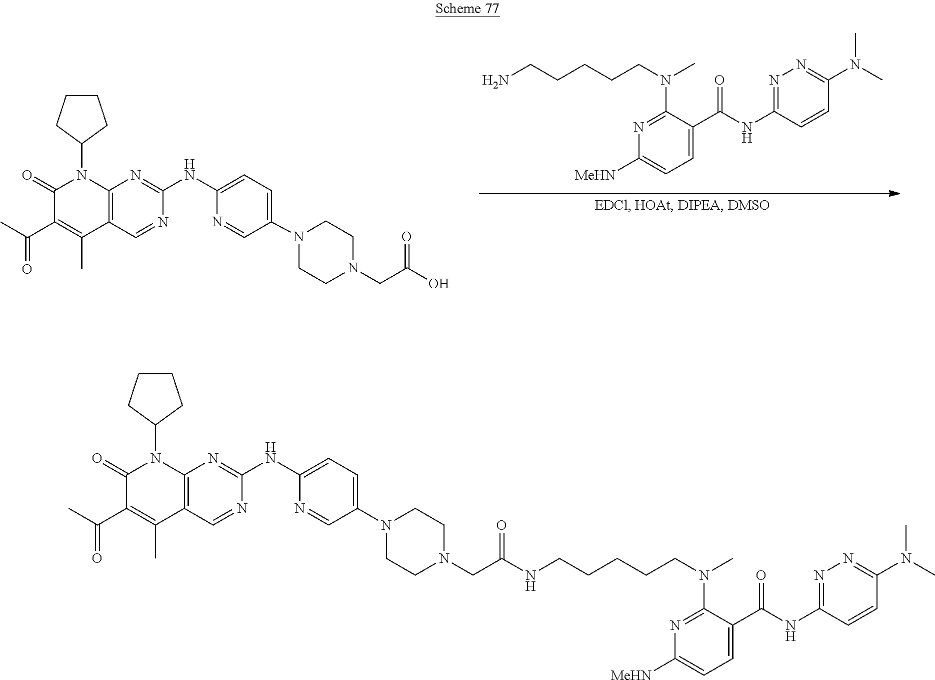 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(N(C)CCCCCN)n1 ; CNc1ccc(C(=O)Nc2ccc(N(C)C)nn2)c(N(C)CCCCCNC(=O)CN2CCN(c3ccc(Nc4ncc5c(C)c(C(C)=O)c(=O)n(C6CCCC6)c5n4)nc3)CC2)n1]

CPD-23 was synthesized following the standard procedure for preparing CPD-1 (8.1 mg, 32% yield) as a yellow solid. MS (ESI) m/z=874.9 [M+H]⁺.

### Example 78. 2-((1-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)-1H-pyrazol-4-yl)amino)-N-(6-(dimethylamino)pyridazin-3-yl)-6-(methylamino)nicotinamide (CPD-24)

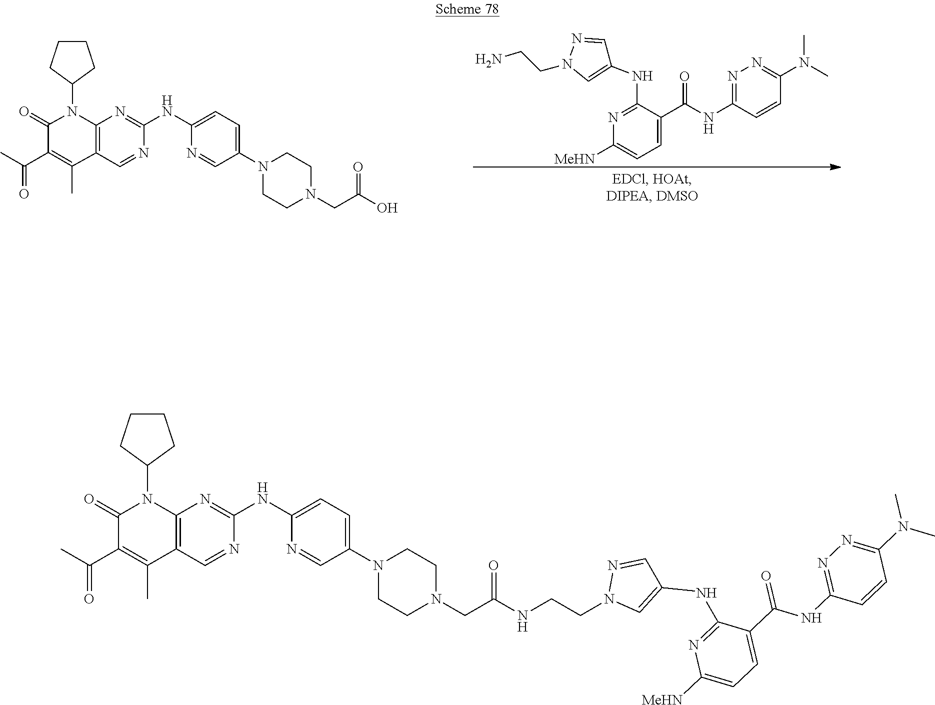

CPD-24 was synthesized following the standard procedure for preparing CPD-1 (2.8 mg, 13% yield) as a yellow solid. MS (ESI) n/z=884.9 [M+H]⁺.

### Example 79. 2-((2-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)-2H-1,2,3-triazol-4-yl)amino)-N-(6-(dimethylamino)pyridazin-3-yl)-6-(methylamino)nicotinamide (CPD-25)

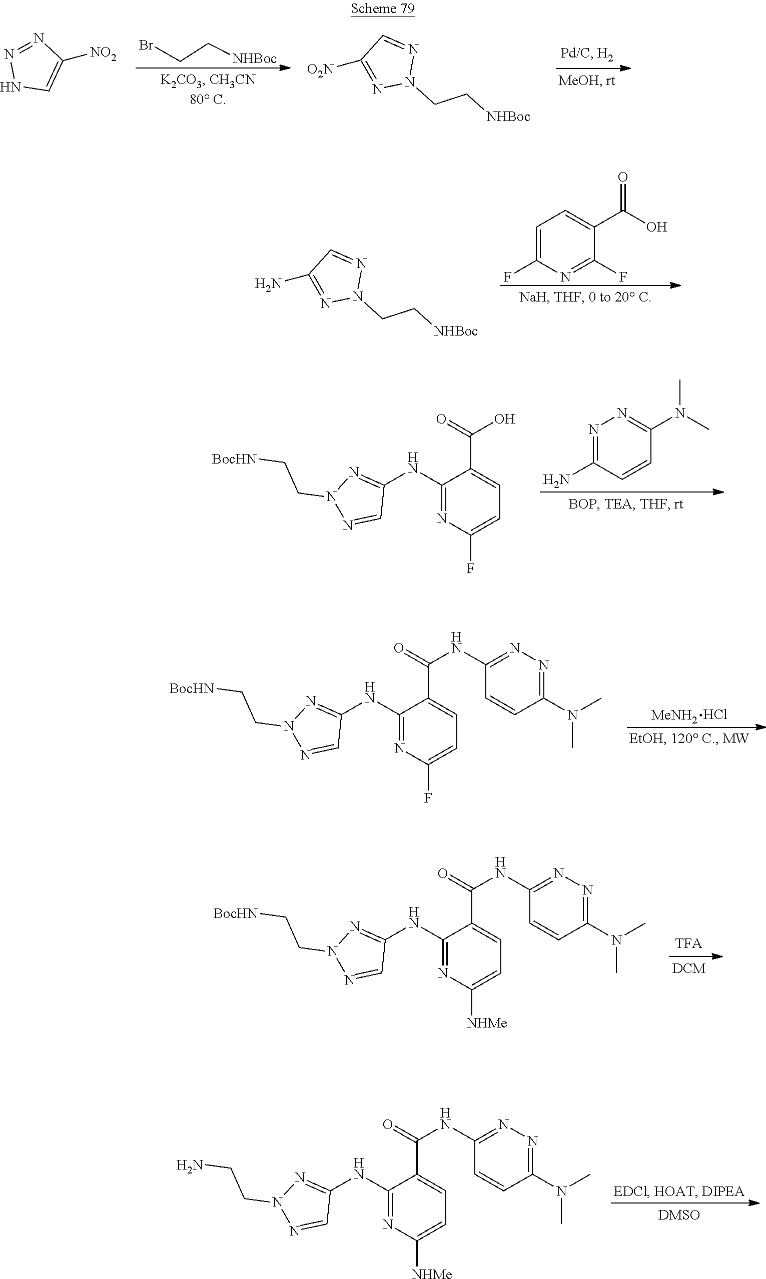

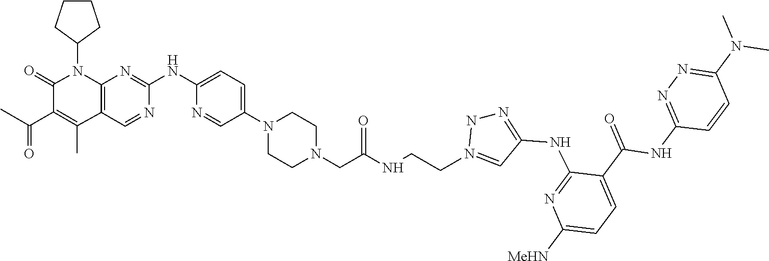

### Step 1. Synthesis of tert-butyl N-[2-(4-nitrotriazol-2-yl)ethyl]carbamate

To a solution of 4-nitro-1H-triazole (2.00 g, 17.5 mmol) in CH₃CN (40 mL) were added K₂CO₃ (7.20 g, 52.1 mmol) and tert-butyl N-(2-bromoethyl) carbamate (4.00 g, 17.8 mmol) at rt. The reaction mixture was stirred at 80 [symbol] for 1 h. After cooling down to rt, the mixture was filtered and the filter cake was washed with EtOAc (50 mL). The filtrate was combined and concentrated. The residue was purified by silica gel column chromatography (petroleum ether/ethyl acetate=10:1 to 3:1) to provide the desired product (1.70 g, 38% yield) as a yellow oil. ¹HNMR (400 MHz, CDCl₃) δ 8.32 (s, 1H), 4.83 (s, 1H), 4.62 (t, J=5.2 Hz, 2H), 3.69 (q, J=6.0 Hz, 2H), 1.43 (s, 9H).

### Step 2. Synthesis of tert-butyl N-[2-(4-aminotriazol-2-yl)ethyl]carbamate

To a solution of tert-butyl N-[2-(4-nitrotriazol-2-yl)ethyl]carbamate (400 mg, 1.55 mmol) in MeOH (5 mL) was added 10% Pd/C (0.10 g) at rt. The reaction mixture was stirred at rt for 4 h under H₂ (15 psi).

The reaction mixture was filtered through Celite. The filtrate was concentrated to provide the desired product (316 mg, 89% yield) as a yellow oil. ¹H NMR (400 MHz, CDCl₃) δ 6.99 (s, 1H), 5.02 (s, 1H), 4.33-4.30 (m, 2H), 3.70 (s, 2H), 3.64-3.60 (m, 2H), 1.44 (s, 9H).

### Step 3. 2-[[2-[2-(tert-butoxycarbonylamino)ethyl]triazol-4-yl]amino]-6-fluoro-pyridine-3-carboxylic acid

To a solution of tert-butyl N-[2-(4-aminotriazol-2-yl)ethyl]carbamate (0.20 g, 0.88 mmol) in THF (1 mL) was added NaH (200 mg, 5.0 mmol, 60% in mineral oil) at 0° C. The mixture was stirred at 0° C. for 0.5 h, then 2,6-difluoropyridine-3-carboxylic acid (200 mg, 1.26 mmol) was added at 0 [symbol] . The reaction mixture was stirred at rt for additional 16 h, before it was quenched with NH₄Cl solution and extracted with ethyl acetate (10 mL×3). The combined organic layers were washed with brine (10 mL), dried with Na₂SO₄, filtered, and concentrated under reduced pressure. The residue was purified by prep-HPLC to provide the desired product (40 mg, 12% yield) as a yellow solid. ¹HNMR (400 MHz, DMSO-d₆) δ 10.91 (s, 1H), 8.41 (t, J=8.4 Hz, 1H), 8.01 (s, 1H), 6.93 (t, J=5.6 Hz, 1H), 6.61 (dd, J=2.8, 8.4 Hz, 1H), 4.34 (t, J=6.0 Hz, 2H), 3.43-3.38 (m, 2H), 1.33 (s, 9H).

### Step 4. Synthesis of tert-butyl N-[2-[4-[[3-[[6-(dimethylamino)pyridazin-3-yl]carbamoyl]-6-fluoro-2-pyridyl]amino]triazol-2-yl]ethyl]carbamate

To a solution of 2-[[2-[2-(tert-butoxycarbonylamino)ethyl]triazol-4-yl]amino]-6-fluoro-pyridine-3-carboxylic acid (60 mg, 0.16 mmol) in THF (1 mL) were added TEA (87.2 mg, 0.86 mmol), BOP (90.0 mg, 0.20 mmol) and N³,N³-dimethylpyridazine-3,6-diamine (30.0 mg, 0.217 mmol) at rt. After the reaction mixture was stirred at rt for 4 h, it was concentrated under reduced pressure. The residue was purified by prep-TLC (petroleum ether/ethyl acetate=0:1) to provide the desired product (23 mg, 28% yield) as a yellow gum. MS (ESI) m/z=487.2 [M+H]⁺.

### Step 5. Synthesis of tert-butyl N-[2-[4-[[3-[[6-(dimethylamino)pyridazin-3-yl]carbamoyl]-6-(methylamino)-2-pyridyl]amino]triazol-2-yl]ethyl]carbamate

To a solution of tert-butyl N-[2-[4-[[3-[[6-(dimethylamino)pyridazin-3-yl]carbamoyl]-6-fluoro-2-pyridyl]amino]triazol-2-yl]ethyl]carbamate (23 mg, 0.047 mmol) in EtOH (1 mL) were added methylamine hydrochloride (100 mg, 1.48 mmol) and TEA (72.7 mg, 0.72 mmol) at rt. The reaction mixture was stirred at 120 [symbol] under microwave irradiation for 2 h. After cooling down to rt, the mixture was poured into H₂O (10 mL) and extracted with EtOAc (15 mL×2). The combined organic layers were washed with brine (20 mL), dried over Na₂SO₄, filtered, and concentrated to provide the desired product (25 mg, 95% yield) as a yellow solid. ¹HNMR (400 MHz, CDCl₃) δ 11.30 (s, 1H), 8.60-8.49 (m, 1H), 8.28-8.25 (m, 1H), 8.20 (s, 1H), 7.75 (d, J=8.4 Hz, 1H), 6.93 (d, J=10.0 Hz, 1H), 5.94 (d, J=8.8 Hz, 1H), 5.03-4.91 (m, 2H), 4.43 (t, J=4.8 Hz, 2H), 3.70-3.67 (m, 2H), 3.16 (s, 6H), 3.07 (d, J=5.2 Hz, 3H), 1.45 (s, 9H). MS (ESI) m/z=498.2 [M+H]⁺.

### Step 6. Synthesis of 2-((2-(2-aminoethyl)-2H-1,2,3-triazol-4-yl)amino)-N-(6-(dimethylamino)pyridazin-3-yl)-6-(methylamino)nicotinamide

To a solution of tert-butyl (2-(4-((3-((6-(dimethylamino)pyridazin-3-yl)carbamoyl)-6-(methylamino)pyridin-2-yl)amino)-2H-1,2,3-triazol-2-yl)ethyl)carbamate (10 mg, 0.02 mmol) in DCM (2 mL) was added TFA (1 mL) at 0° C. After stirring at rt for 1 h, the solution was concentrated under reduced pressure to provide the desired product (10 mg, 97% yield) as TFA salt. MS (ESI) m/z=398.5 [M+H]⁺.

### Step 7. Synthesis of 2-((2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)-2H-1,2,3-triazol-4-yl)amino)-N-(6-(dimethylamino)pyridazin-3-yl)-6-(methylamino)nicotinamide

The title compound was synthesized following the procedure for preparing CPD-1 (4.6 mg, 24% yield) as a yellow solid. MS (ESI) m/z=885.8 [M+H]⁺.

### Example 80. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)isoxazol-5-yl)amino)-N-(6-(dimethylamino)pyridazin-3-yl)-6-(methylamino)nicotinamide (CPD-26)

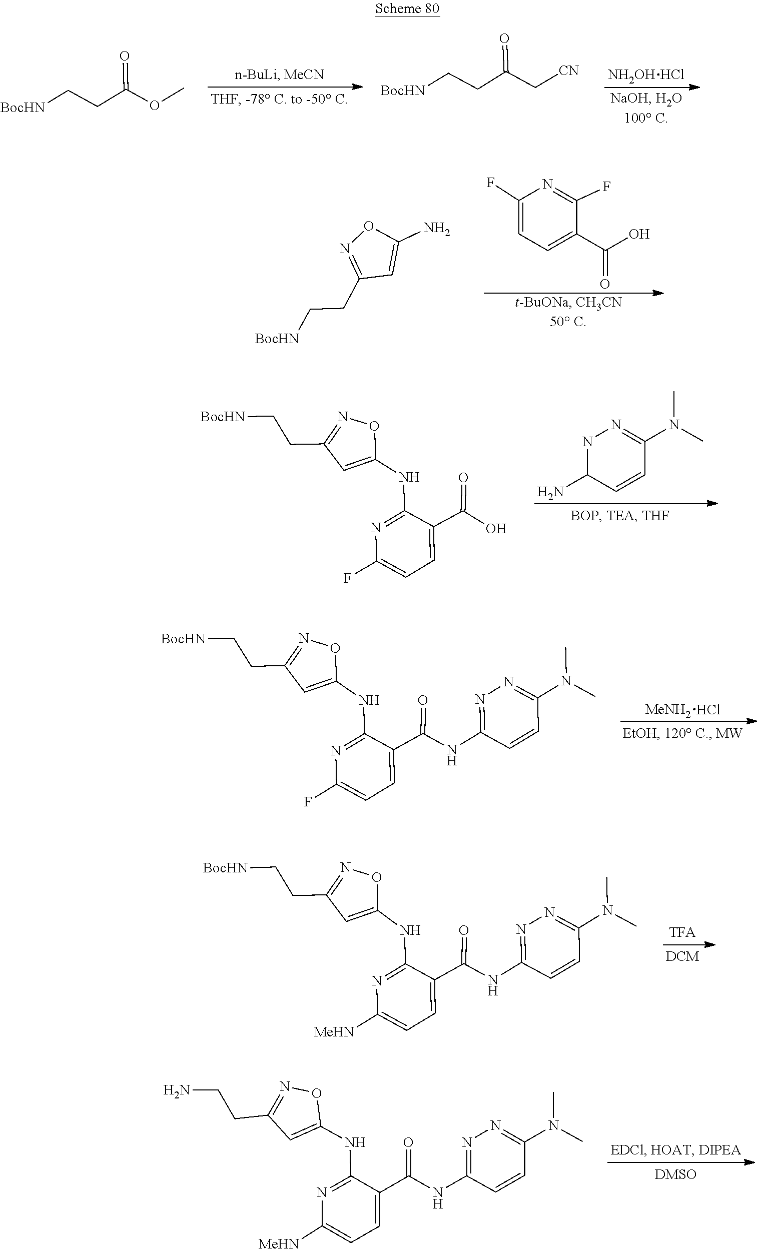

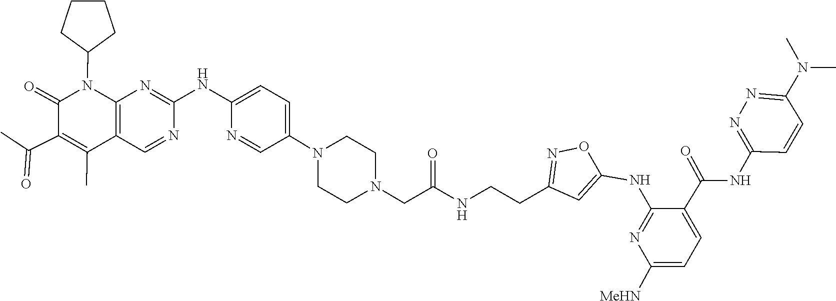

### Step 1. Synthesis of tert-butyl N-(4-cyano-3-oxo-butyl)carbamate

To a solution of n-BuLi (2.5 M in THF, 100 mL) in THF (200 mL) was dropwise a solution of CH₃CN (12.4 g, 304 mmol) in THF (20 mL) at −78 [symbol] . The reaction mixture was stirred at −78 [symbol] for 1 h, then a solution of methyl 3-(tert-butoxycarbonylamino) propanoate (22.0 g, 108 mmol) in THF (20 mL) was dropwise to the mixture at −78 [symbol] . After the reaction mixture was stirred at −50 [symbol] for additional 1 h, it was quenched with AcOH (100 mL), H₂O (200 mL) at −10~0 [symbol] slowly. The solution was extracted with EtOAc (100 mL×3). The combined organic layers were washed with brine (200 mL), dried over Na₂SO₄, filtered, and concentrated to provide the desired product (23.0 g, crude) as a brown oil. ¹HNMR (400 MHz, CDCl₃) δ 3.54 (s, 2H), 3.40-3.36 (m, 2H), 2.81 (t, J=5.6 Hz, 2H), 1.41 (s, 9H).

### Step 2. Synthesis of tert-butyl N-[2-(5-aminoisoxazol-3-yl)ethyl]carbamate

To a solution of tert-butyl N-(4-cyano-3-oxo-butyl) carbamate (20.0 g, 94.2 mmol) in H₂O (400 mL) was added NaOH (10.0 g, 250 mmol) and NH₂OH·HCl (8.00 g, 115 mmol) at rt. The reaction mixture was stirred at 100 [symbol] for 3 h. After cooling down to rt, the solution was filtered and the filtrate was extracted with EtOAc (200 mL×3). The combined organic layers were washed with brine (200 mL), dried over Na₂SO₄, filtered, and concentrated to provide the desired product (6.50 g, 30% yield) as a yellow solid. ¹HNMR (400 MHz, CDCl₃) J=5.00 (s, 1H), 4.91 (s, 1H), 4.59 (s, 2H), 3.44-3.39 (m, 2H), 2.70 (t, J=6.4 Hz, 2H), 1.42 (s, 9H)

### Step 3. Synthesis of 2-[[3-[2-(tert-butoxycarbonylamino)ethyl]isoxazol-5-yl]amino]-6-fluoro-pyridine-3-carboxylic acid

To a solution of tert-butyl N-[2-(5-aminoisoxazol-3-yl)ethyl]carbamate (3.00 g, 13.2 mmol) in CH₃CN (20 mL) were added t-BuONa (3.00 g, 31.2 mmol) and 2,6-difluoropyridine-3-carboxylic acid (1.50 g, 9.43 mmol) at 25 [symbol] . The reaction mixture was stirred at 50 [symbol] for 16 h. After cooling down to rt, the reaction mixture was concentrated under vacuum. The residue was diluted with H₂O (50 mL), adjusted pH to 3~4 with aq. HCl solution (1N, 25 mL) and extracted with ethyl acetate (50 mL×3). The combined organic layers were washed with brine (100 mL), dried with Na₂SO₄, filtered, and concentrated under reduced pressure. The crude product was purified by prep-HPLC to provide the desired product (0.64 g, 19% yield) as a brown solid. ¹HNMR (400 MHz, DMSO-d₆) δ 11.48 (brs, 1H), 8.48 (t, J=8.4 Hz, 1H), 6.97 (t, J=5.6 Hz, 1H), 6.80 (dd, J=2.8, 8.4 Hz, 1H), 6.33 (s, 1H), 3.22 (q, J=6.8 Hz, 2H), 2.72 (t, J=7.2 Hz, 2H), 1.36 (s, 9H).

The remaining steps were performed according to the procedures of steps 4 to 7 for preparing CPD-25 to provide the desired product (7.3 mg, 16% yield for 4 steps) as a yellow solid. MS (ESI) m/z=885.8 [M+H]⁺.

### Example 81. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyclopropyl-N-(6-(dimethylamino)pyridazin-3-yl)nicotinamide (CPD-27)

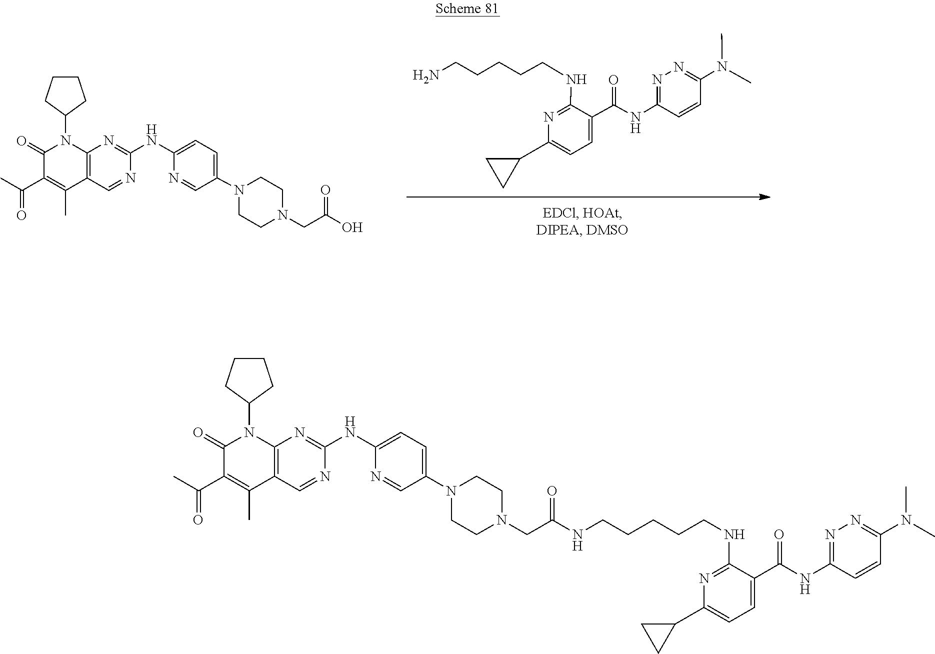 [SMILES: CC(=O)c1c(C)c2cnc(Nc3ccc(N4CCN(CC(=O)O)CC4)cn3)nc2n(C2CCCC2)c1=O ; CN(C)c1ccc(NC(=O)c2ccc(C3CC3)nc2NCCCCCN)nn1 ; CC(=O)c1c(C)c2cnc(Nc3ccc(N4CCN(CC(=O)NCCCCCNc5nc(C6CC6)ccc5C(=O)Nc5ccc(N(C)C)nn5)CC4)cn3)nc2n(C2CCCC2)c1=O]

CPD-27 was synthesized following the standard procedure for preparing CPD-1 (12.6 mg, 52% yield) as a yellow solid. MS (ESI) m/z=871.8 [M+H]⁺.

### Example 82. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyano-N-(6-(dimethylamino)pyridazin-3-yl)nicotinamide (CPD-28)

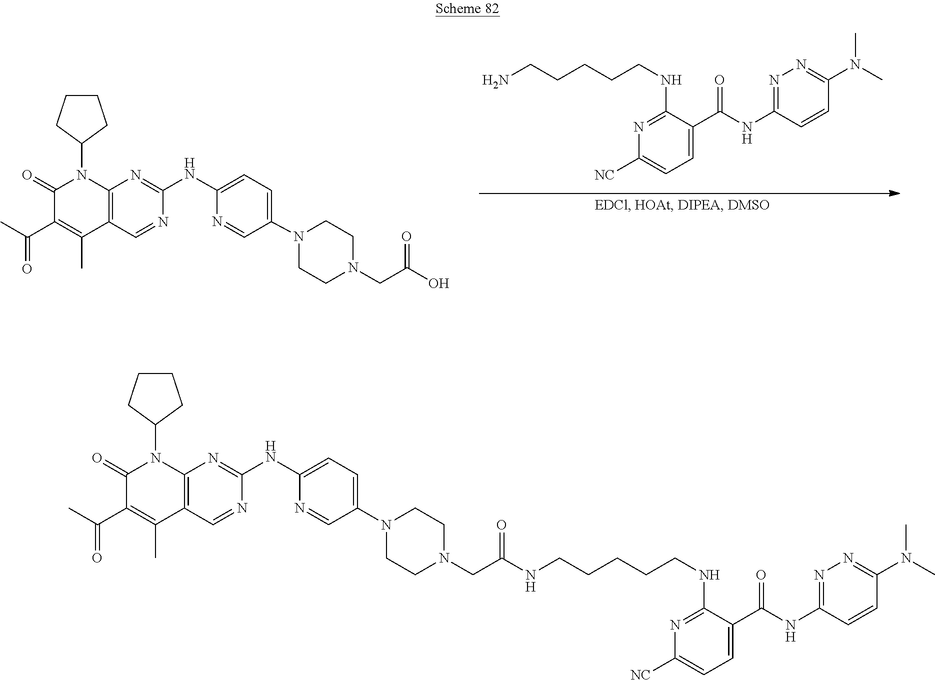 [SMILES: CC(=O)c1c(C)c2cnc(Nc3ccc(N4CCN(CC(=O)O)CC4)cn3)nc2n(C2CCCC2)c1=O ; CN(C)c1ccc(NC(=O)c2ccc(C#N)nc2NCCCCCN)nn1 ; CC(=O)c1c(C)c2cnc(Nc3ccc(N4CCN(CC(=O)NCCCCCNc5nc(C#N)ccc5C(=O)Nc5ccc(N(C)C)nn5)CC4)cn3)nc2n(C2CCCC2)c1=O]

CPD-28 was synthesized following the standard procedure for preparing CPD-1 (5.4 mg, 25% yield) as a yellow solid. MS (ESI) m/z=856.8 [M+H]⁺.

### Example 83. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexan-2-yl)amino)-N-(6-(dimethylamino)pyridazin-3-yl)-6-(methylamino)nicotinamide (CPD-29)

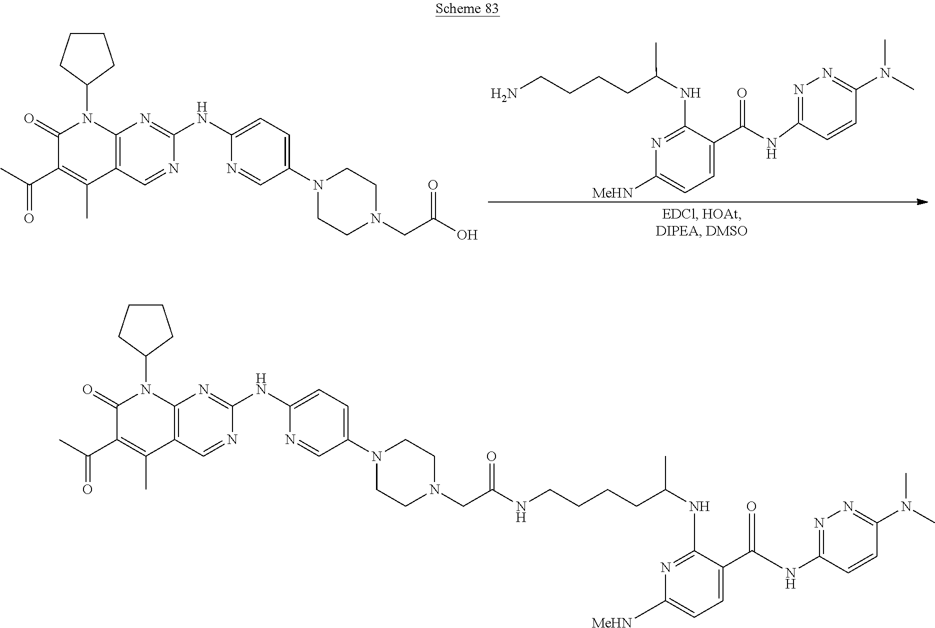 [SMILES: CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(NC(C)CCCCN)n1 ; CNc1ccc(C(=O)Nc2ccc(N(C)C)nn2)c(NC(C)CCCCNC(=O)CN2CCN(c3ccc(Nc4ncc5c(C)c(C(C)=O)c(=O)n(C6CCCC6)c5n4)nc3)CC2)n1]

CPD-29 was synthesized following the standard procedure for preparing CPD-1 (6.1 mg, 28% yield) as a yellow solid. MS (ESI) m/z=874.8 [M+H].

### Example 84. 2-((4-(4-((4-(6-((6-Acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)butyl)amino)-N-(6-(dimethylamino)pyridazin-3-yl)-6-(methylamino)nicotinamide (CPD-30)

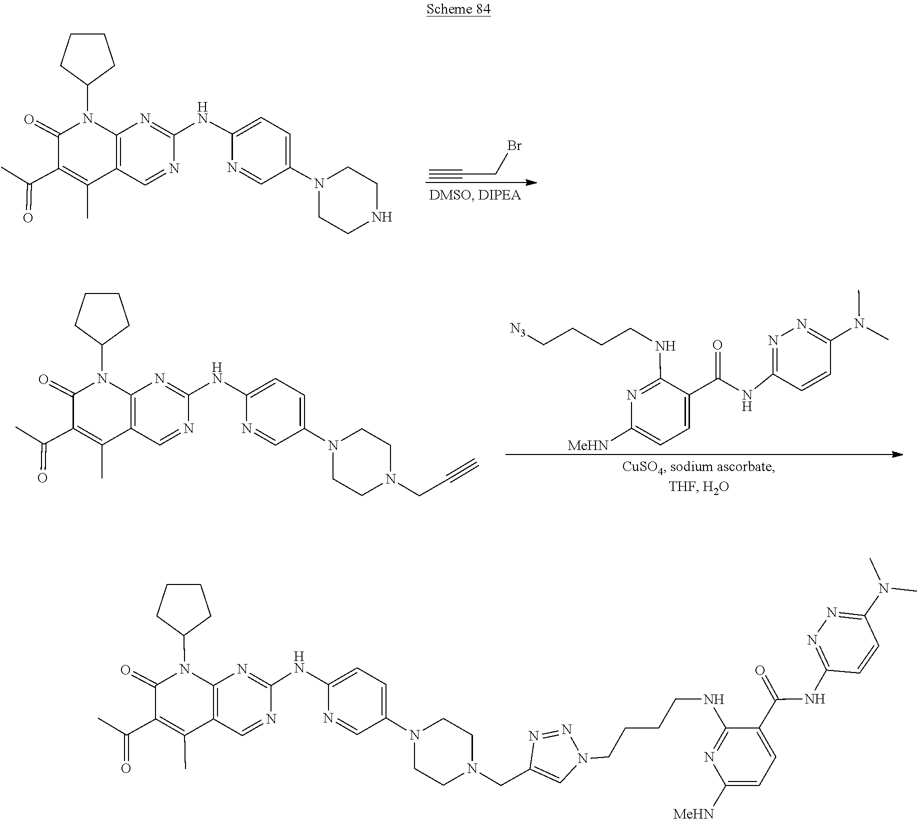

### Step 1. Synthesis of 6-acetyl-8-cyclopentyl-5-methyl-2-((5-(4-(prop-2-yn-1-yl)piperazin-1-yl)pyridin-2-yl)amino)pyrido[2,3-d]pyrimidin-7(8H)-one

To a mixture of 6-acetyl-8-cyclopentyl-5-methyl-2-((5-(piperazin-1-yl)pyridin-2-yl)amino)pyrido[2,3-d]pyrimidin-7(8H)-one (30 mg, 0.06 mmol) and DIPEA (43 mg, 0.3 mmol) in DMSO (2 mL) was added 3-bromoprop-1-yne (10 mg, 0.08 mmol) at rt. The reaction mixture was stirred at 90 [symbol] for 1 h. After cooling down to rt, the reaction was quenched with water (30 mL) and extracted with ethyl acetate (20 mL×2). The combined organic layers were washed with brine, dried over anhydrous sodium sulfate, filtered, and concentrated. The residue was purified by silica gel flash chromatography to provide the desired product (28 mg, 86% yield) as a colorless oil. MS (ESI) m/z=486.6 [M+H]⁺.

### Step 2. Synthesis of 2-((4-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)butyl)amino)-N-(6-(dimethylamino)pyridazin-3-yl)-6-(methylamino)nicotinamide

A mixture of 6-acetyl-8-cyclopentyl-5-methyl-2-[[5-(4-prop-2-ynylpiperazin-1-yl)-2-pyridyl]amino]pyrido[2,3-d]pyrimidin-7-one (12 mg, 0.02 mmol), 2-(4-azidobutylamino)-N-[6-(dimethylamino)pyridazin-3-yl]-6-(methylamino)pyridine-3-carboxamide (10 mg, 0.02 mmol), sodium ascorbate (5 mg, 0.002 mmol) and copper(II) sulfate pentahydrate (6 mg, 0.002 mmol) in THF (2 mL) and H₂O (2 mL) was stirred at rt for 1 h. The reaction mixture was purified by reverse-phase chromatography and prep-TLC to provide the desired product (7.2 mg, 28% yield) as a yellow solid. MS (ESI) m/z=870.9 [M+H]⁺.

### Example 85. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridazin-3-yl)-6-(methylamino)nicotinamide (CPD-31)

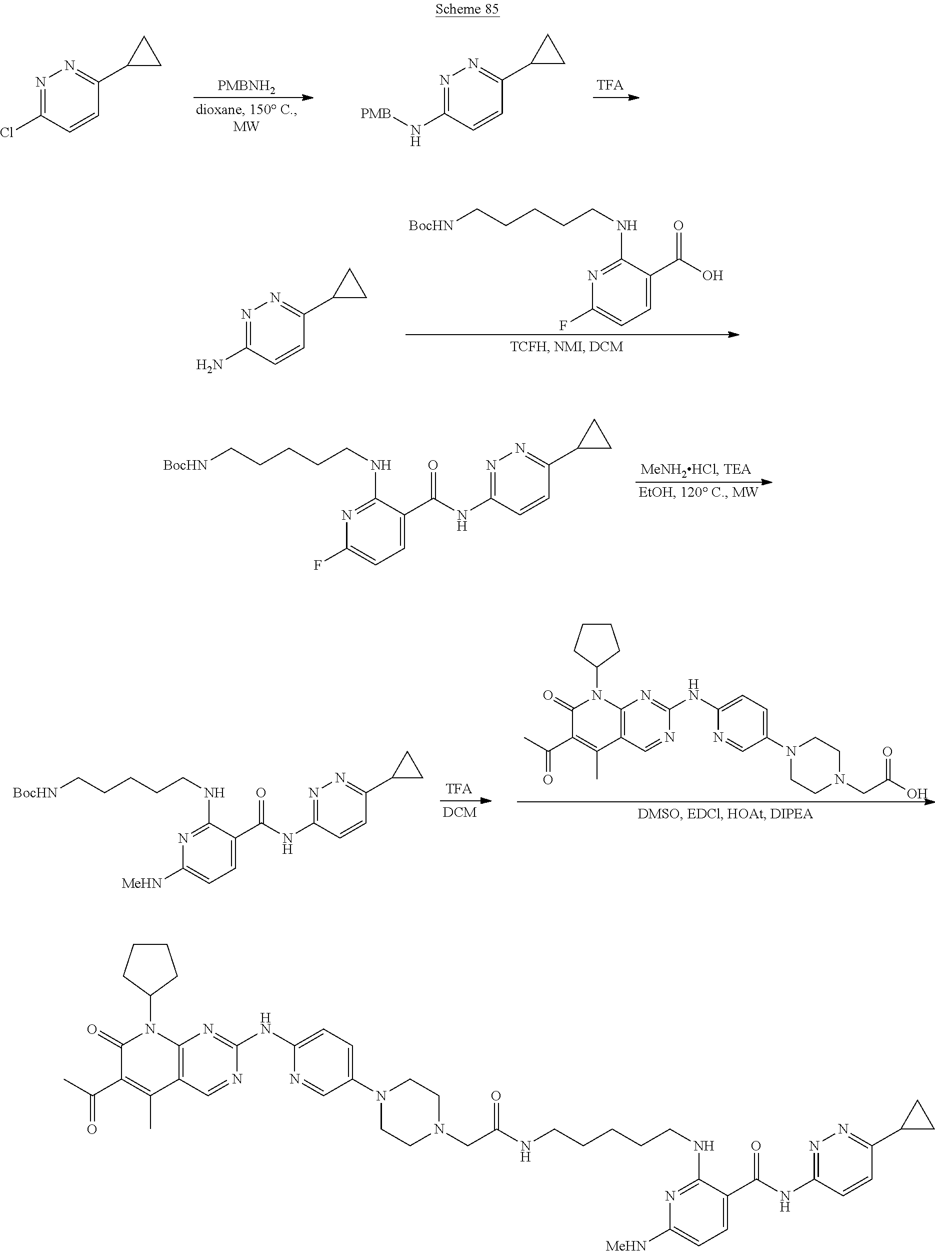

### Step 1. Synthesis of 6-cyclopropyl-N-[(4-methoxyphenyl)methyl]pyridazin-3-amine

A solution of 3-chloro-6-cyclopropyl-pyridazine (0.27 g, 1.75 mmol), DIEA (742 mg, 5.74 mmol) and [symbol] (4-methoxyphenyl) methanamine (3.18 g, 23.1 mmol) in dioxane (0.5 mL) was stirred at 150° C. under microwave irradiation for 4 h. After cooling down to rt, the mixture was poured into H₂O (10 mL) and extracted with EtOAc (20 mL×2). The combined organic layers were washed with brine (20 mL), dried over Na₂SO₄, filtered, and concentrated. The residue was purified by silica gel column chromatography (petroleum ether/ethyl acetate=0:1 to 1:0) to provide the desired product (0.21 g, 47% yield) as a yellow oil. ¹HNMR (400 MHz, CDCl₃) δ 7.33-7.28 (m, 2H), 6.95 (d, J=9.2 Hz, 1H), 6.87 (d, J=8.8 Hz, 2H), 6.55 (d, J=8.8 Hz, 1H), 4.53 (d, J=5.6 Hz, 2H), 3.80 (s, 3H), 2.04-1.97 (m, 1H), 1.06-0.94 (m, 4H)

### Step 2. Synthesis of 6-cyclopropylpyridazin-3-amine

A solution of 6-cyclopropyl-N-[(4-methoxyphenyl)methyl]pyridazin-3-amine (0.21 g, 0.82 mmol) in TFA (5 mL) was stirred at rt for 12 h. The reaction mixture was slowly poured into saturated NaHCO₃ solution (20 mL) and adjusted pH to 11~12 with aq. NaOH solution (2N, 4 mL). The aqueous phase was extracted with EtOAc (20 mL×5). The combined organic layers were washed with brine (30 mL), dried over Na₂SO₄, filtered, and concentrated. The residue was purified by prep-TLC (dichloromethane/Methanol=10:1) to provide the desired product (60 mg, 54% yield) as a yellow solid. ¹H NMR (400 MHz, CDCl₃) δ 7.01 (d, J=9.2 Hz, 1H), 6.72 (d, J=9.2 Hz, 1H), 4.77 (s, 2H), 2.05 (m, 1H), 1.01 (d, J=6.4 Hz, 4H)

### Step 3. Synthesis of tert-butyl N-[5-[[3-[(6-cyclopropylpyridazin-3-yl)carbamoyl]-6-fluoro-2-pyridyl]amino]pentyl]carbamate

To a solution of 2-[5-(tert-butoxycarbonylamino)pentylamino]-6-fluoro-pyridine-3-carboxylic acid (0.15 g, 0.44 mmol) in DCM (2 mL) were added 1-methylimidazole (103 mg, 1.25 mmol), 6-cyclopropylpyridazin-3-amine (48 mg, 0.36 mmol) and TCFH (150 mg, 0.53 mmol) at rt. The reaction mixture was stirred at rt for 15 h. The reaction mixture was concentrated and purified by prep-TLC (petroleum ether/ethyl acetate=1:1) to provide the desired product (55 mg, 27% yield) as a yellow solid. ¹HNMR (400 MHz, CDCl₃) δ 9.19-8.97 (m, 1H), 8.52 (s, 1H), 8.34-8.26 (m, 1H), 8.05 (t, J=7.6 Hz, 1H), 6.13 (dd, J=2.8, 8.4 Hz, 1H), 4.53 (s, 1H), 3.52-3.45 (m, 2H), 3.17-3.09 (m, 2H), 2.23-2.11 (m, 1H), 1.70-1.65 (m, 2H), 1.56-1.51 (m, 2H), 1.44 (s, 11H), 1.13 (t, J=6.4 Hz, 4H).

The remaining steps were performed according to the procedures of steps 5 to 7 for preparing CPD-25 to provide the desired product (1.2 mg, 5% yield for 3 steps) as a yellow solid. MS (ESI) m/z=857.8 [M+H]⁺.

### Example 86. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrazolo[4,3-bipyridin-5-yl)-6-(methylamino)nicotinamide (CPD-32)

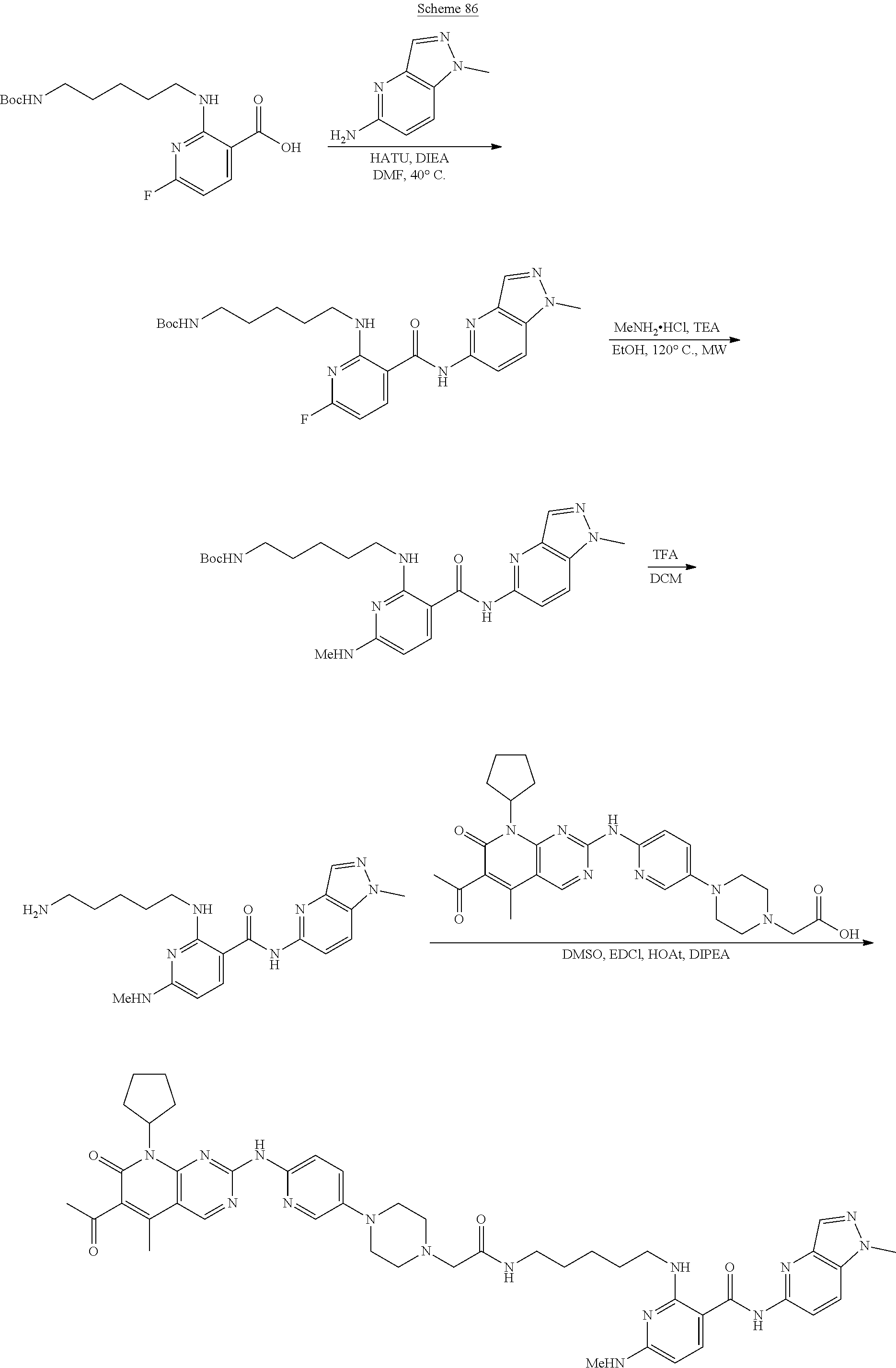

### Step 1. Synthesis of tert-butyl N-[5-[[6-fluoro-3-[(1-methylpyrazolo[4, 3-b]pyridin-5-yl) carbamoyl]-2-pyridyl]amino]pentyl]carbamate

To a solution of 2-[5-(tert-butoxycarbonylamino)pentylamino]-6-fluoro-pyridine-3-carboxylic acid (350 mg, 1.03 mmol) and 1-methylpyrazolo[4,3-b]pyridin-5-amine (230 mg, 1.55 mmol) in DMF (3 mL) were added DIEA (519 mg, 4.02 mmol) and HATU (510 mg, 1.34 mmol) at rt. After the reaction mixture was stirred at 40° C. for 12 h, it was poured into H₂O (20 mL) and extracted with EtOAc (10 mL×3). The combined organic layers were washed with brine (15 mL×3), dried over Na₂SO₄, filtered, and concentrated. The crude product was purified by pre-TLC (petroleum ether/ethyl acetate=1:1) to provide the desired product (305 mg, 49% yield) as a yellow solid. MS (ESI) m/z=472.3 [M+H]⁺.

The remaining steps were performed according to the procedures of steps 5 to 7 for preparing CPD-25 to provide the desired product (1.2 mg, 2% yield for 3 steps) as a yellow solid. MS (ESI) m/z=870.8 [M+H]⁺.

### Example 87. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrrolo[3,2-b]pyridin-5-yl)-6-(methylamino)nicotinamide (CPD-33)

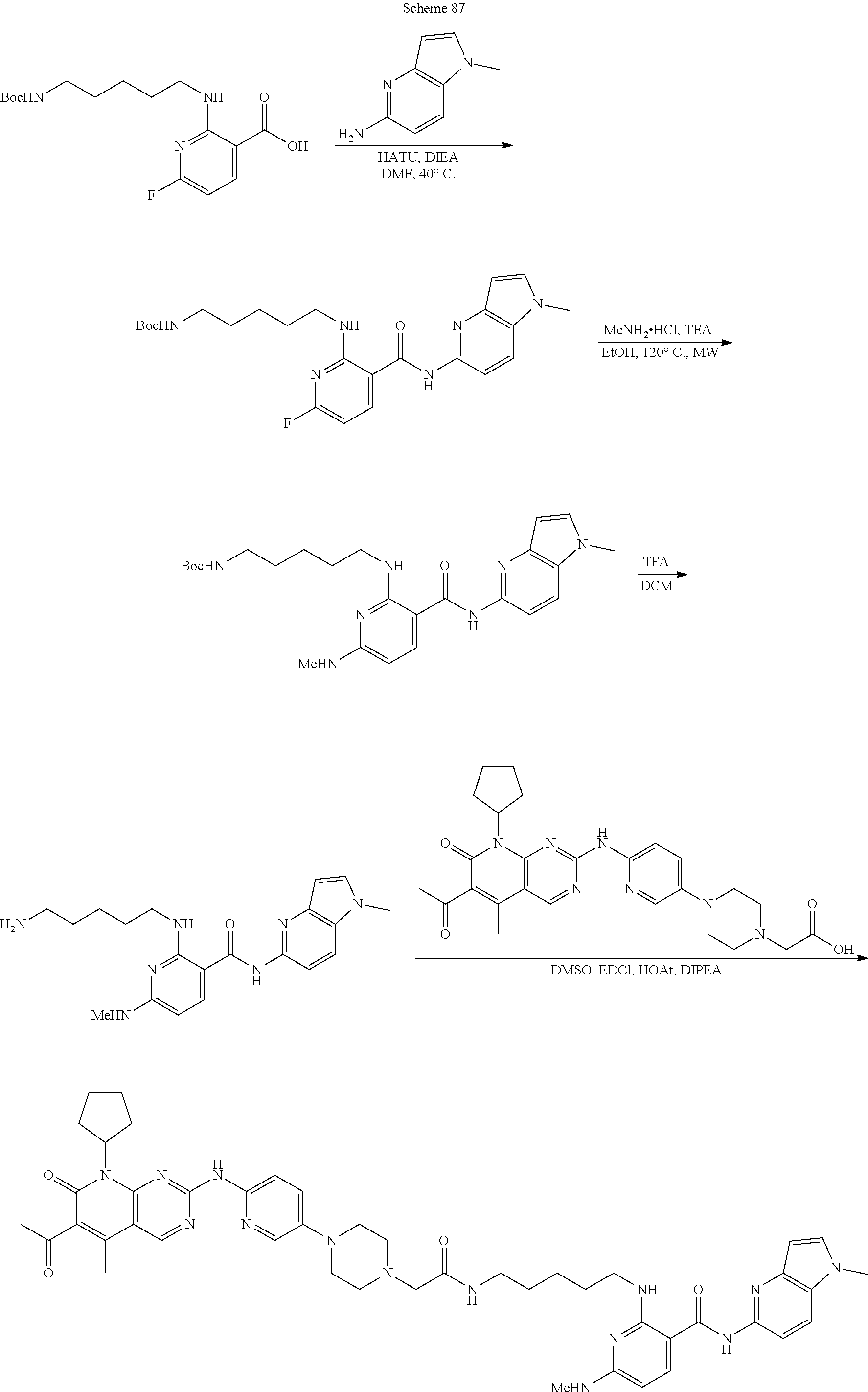

### Step 1. Synthesis of tert-butyl N-[5-[[6-fluoro-3-[(1-methylpyrrolo[3, 2-b]pyridin-5-yl) carbamoyl]-2-pyridyl]amino]pentyl]carbamate

The title compound was synthesized following the procedure of step 1 for preparing CPD-32 (180 mg, 61% yield) as a yellow solid. ¹H NMR (400 MHz, MeOH-d₄) δ 8.22 (t, J=8.0 Hz, 1H), 7.90 (d, J=8.8 Hz, 1H), 7.79 (d, J=9.2 Hz, 1H), 7.47 (d, J=3.2 Hz, 1H), 6.49 (d, J=3.2 Hz, 1H), 6.17 (dd, J=2.8, 8.4 Hz, 1H), 3.87 (s, 3H), 3.45 (t, J=7.2 Hz, 2H), 3.05 (q, J=7.2 Hz, 2H), 1.68-1.61 (m, 2H), 1.55-1.49 (m, 2H), 1.47-1.43 (m, 2H), 1.41 (s, 9H). MS (ESI) m/z=471.5 [M+H]⁺.

The remaining steps were performed according to the procedures of steps 5 to 7 for preparing CPD-25 to provide the desired product (7.8 mg, 19% yield for 3 steps) as a yellow solid. MS (ESI) m/z=869.9 [M+H]⁺.

### Example 88. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(6-(dimethylamino)pyridazin-3-yl)-6-(methylamino)nicotinamide (CPD-34)

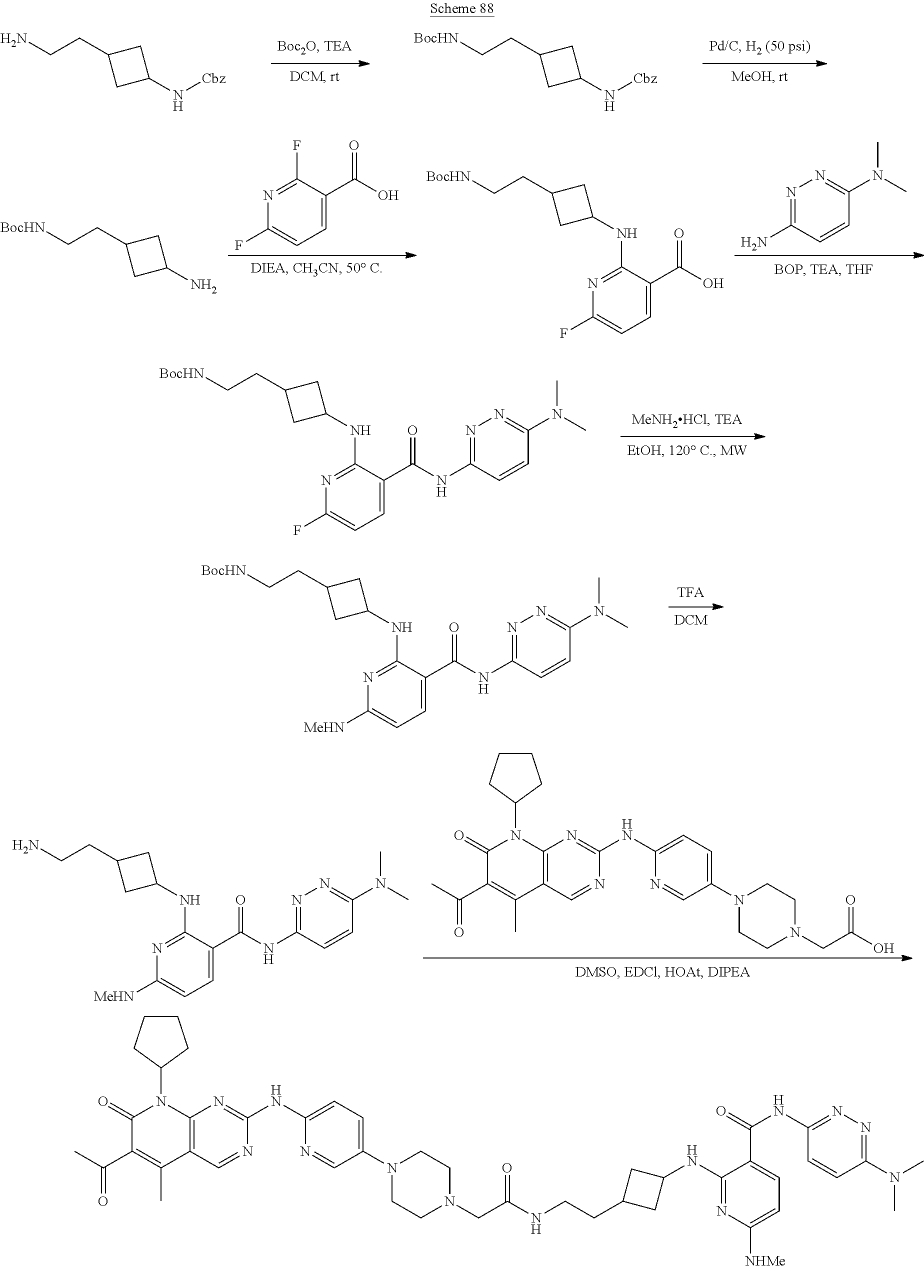

### Step 1. Synthesis of tert-butyl N-[2-[3-(benzyloxycarbonylamino)cyclobutyl]ethyl]carbamate

To a solution of benzyl N-[3-(2-aminoethyl)cyclobutyl]carbamate (0.40 g, 1.61 mmol) in DCM (4 mL) were added TEA (509 mg, 5.03 mmol) and (Boc)₂O (380 mg, 1.74 mmol) at rt. The reaction mixture was stirred at rt for 5 h, before it was poured into H₂O (20 mL) and adjusted pH to 3~4 with aq. HCl (0.5 N, 6 mL). The aqueous layer was extracted with DCM (20 mL×2). The combined organic layers were washed with brine (30 mL), dried over Na₂SO₄, filtered, and concentrated to provide the desired product (0.48 g, 85% yield) as a yellow oil. ¹HNMR (400 MHz, CDCl₃) δ 7.41-7.29 (m, 5H), 5.08 (s, 2H), 4.98-4.77 (m, 1H), 4.47 (s, 1H), 4.30-3.97 (m, 1H), 3.06 (s, 2H), 2.52 (ds, 1H), 2.26-1.85 (m, 3H), 1.70-1.64 (m, 1H), 1.59-1.51 (m, 2H), 1.44 (s, 9H).

### Step 2. Synthesis of tert-butyl N-[2-(3-aminocyclobutyl)ethyl]carbamate

To a mixture of tert-butyl N-[2-[3-(benzyloxycarbonylamino)cyclobutyl]ethyl]carbamate (0.43 g, 1.23 mmol) in MeOH (5 mL) was added 10% Pd/C (0.10 g, 1.23 mmol) at rt. The reaction mixture was stirred at rt for 5 h under H₂ (50 psi). The reaction mixture was filtered through Celite. The filtrate was concentrated to provide the desired product (0.24 g, 91% yield) as a colorless oil. ¹HNMR (400 MHz, CDCl₃) δ 4.52 (s, 1H), 3.10-2.96 (m, 2H), 2.49-2.37 (m, 1H), 2.29-2.12 (m, 1H), 2.07-1.96 (m, 3H), 1.90-1.80 (m, 1H), 1.65-1.50 (m, 2H), 1.43 (s, 9H), 1.37-1.23 (m, 1H).

### Step 3. Synthesis of 2-[[3-[2-(tert-butoxycarbonylamino)ethyl]cyclobutyl]amino]-6-fluoro-pyridine-3-carboxylic acid

To a mixture of tert-butyl N-[2-(3-aminocyclobutyl)ethyl]carbamate (0.24 g, 1.12 mmol) and 2,6-difluoropyridine-3-carboxylic acid (0.23 g, 1.45 mmol) in CH₃CN (3 mL) was added DIEA (445 mg, 3.44 mmol) at rt. The reaction mixture was stirred at 50 [symbol] for 12 h. After cooling down to rt, the reaction mixture was concentrated in vacuum. The residue was purified by prep-TLC (dichloromethane/methanol=10:1) to provide the desired product (350 mg, 85% yield) as a yellow oil. MS (ESI) m/z=298.3 [M+H-56].

The remaining steps were performed according to the procedures of steps 4 to 7 for preparing CPD-25 to provide the desired product (8.1 mg, 1% yield for 4 steps) as a yellow solid. MS (ESI) m/z=872.8 [M+H]⁺.

### Example 89. 4-(6-((6-Acetyl-8-cyclopentyl-5-methyl-7-oxo-7,8-dihydropyrido[2,3-d]pyrimidin-2-yl)amino)pyridin-3-yl)-N-(2-(3-((3-((6-(dimethylamino)pyridazin-3-yl)carbamoyl)-6-(methylamino)pyridin-2-yl)amino)cyclobutyl)ethyl)piperazine-1-carboxamide (CPD-35)

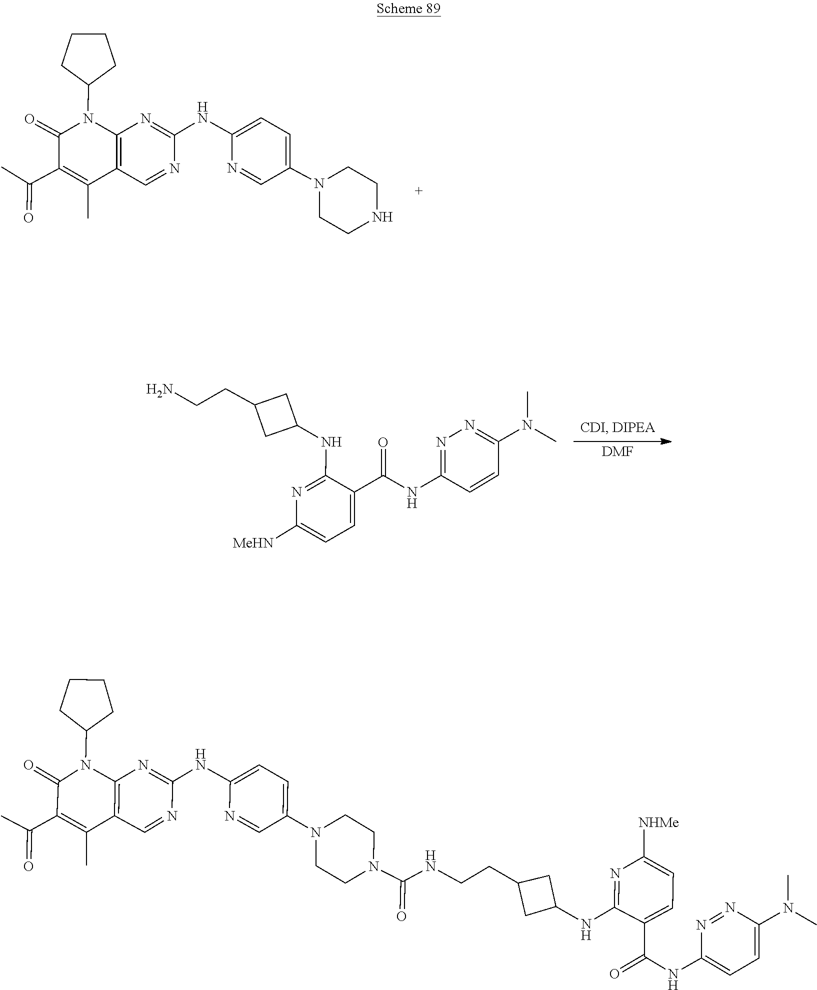

To a mixture of 6-acetyl-8-cyclopentyl-5-methyl-2-((5-(piperazin-1-yl)pyridin-2-yl)amino)pyrido[2,3-d]pyrimidin-7(8H)-one (9 mg, 0.02 mmol) and 2-((3-(2-aminoethyl)cyclobutyl)amino)-N-(6-(dimethylamino)pyridazin-3-yl)-6-(methylamino)nicotinamide (7 mg, 0.015 mmol) in DMF (2 mL) were added CDI (4 mg, 0.02 mmol) and DIPEA (4 mg, 0.03 mmol) at rt. After the reaction mixture was stirred at rt for 16 h, it was poured into water (20 mL) and extracted with ethyl acetate (3×20 mL). The combined organic layers were washed with saturated brine (20 mL), dried over anhydrous sodium sulfate, filtered, and evaporated under reduced pressure. The residue was purified by reverse-phase chromatography to provide the desired product (6.0 mg, 44% yield) as a yellow solid. MS (ESI) m/z=858.8 [M+H]⁺.

### Example 90. 2-((5-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)-5-oxopentyl)amino)-N-(6-(dimethylamino)pyridazin-3-yl)-6-(methylamino)nicotinamide (CPD-36)

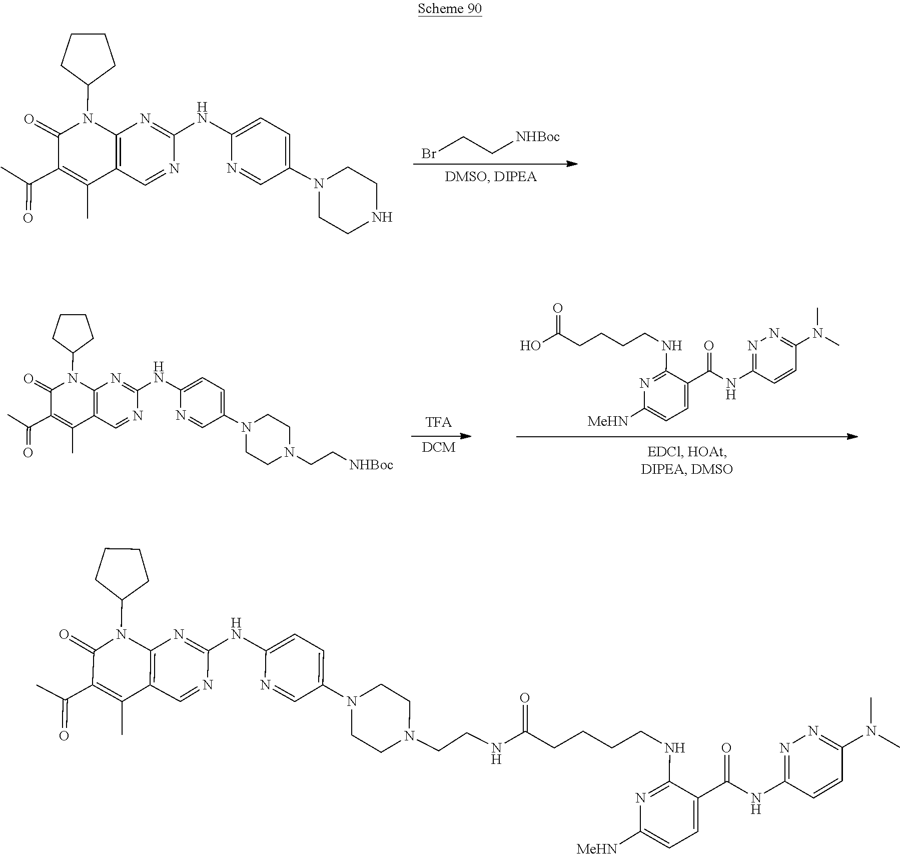

### Step 1. Synthesis of tert-butyl (2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamate

To a mixture of 6-acetyl-8-cyclopentyl-5-methyl-2-((5-(piperazin-1-yl)pyridin-2-yl)amino)pyrido[2,3-d]pyrimidin-7(8H)-one (30 mg, 0.06 mmol) and DIPEA (26 mg, 0.2 mmol) in DMSO (2 mL) was added tert-butyl (2-bromoethyl)carbamate (15 mg, 0.06 mmol) at rt. The reaction mixture was stirred at 100 [symbol] for 2 h. After the reaction mixture was cooled down to rt, it was poured into water (20 mL) and extracted with DCM (3×20 mL). The combined organic layers were washed with saturated brine (20 mL), dried over anhydrous sodium sulfate, filtered, and evaporated under reduced pressure. The residue was purified by silica gel flash chromatography to provide the desired product (21 mg, 44% yield) as a colorless oil. MS (ESI) m/z=591.7 [M+H]⁺.

### Step 2. Synthesis of 6-acetyl-2-((5-(4-(2-aminoethyl)piperazin-1-yl)pyridin-2-yl)amino)-8-cyclopentyl-5-methylpyrido[2,3-d]pyrimidin-7(8H)-one

A solution of tert-butyl (2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamate (21 mg, 0.03 mmol) in TFA (1 mL) and DCM (1 mL) was stirred at rt for 1 h. The reaction mixture was concentrated and purified by reverse-phase chromatography to provide the desired product (17 mg, 79% yield) as a colorless oil. MS (ESI) m/z=491.6 [M+H]⁺.

### Step 3. Synthesis of 2-((5-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)amino)-5-oxopentyl)amino)-N-(6-(dimethylamino)pyridazin-3-yl)-6-(methylamino)nicotinamide

The title compound was synthesized following the procedure for preparing CPD-1 (8.7 mg, 35% yield) as a light-yellow solid. MS (ESI) m/z=860.8 [M+H]⁺.

### Example 91. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)butyl)amino)-N-(6-(dimethylamino)pyridazin-3-yl)-6-(methylamino)nicotinamide (CPD-37)

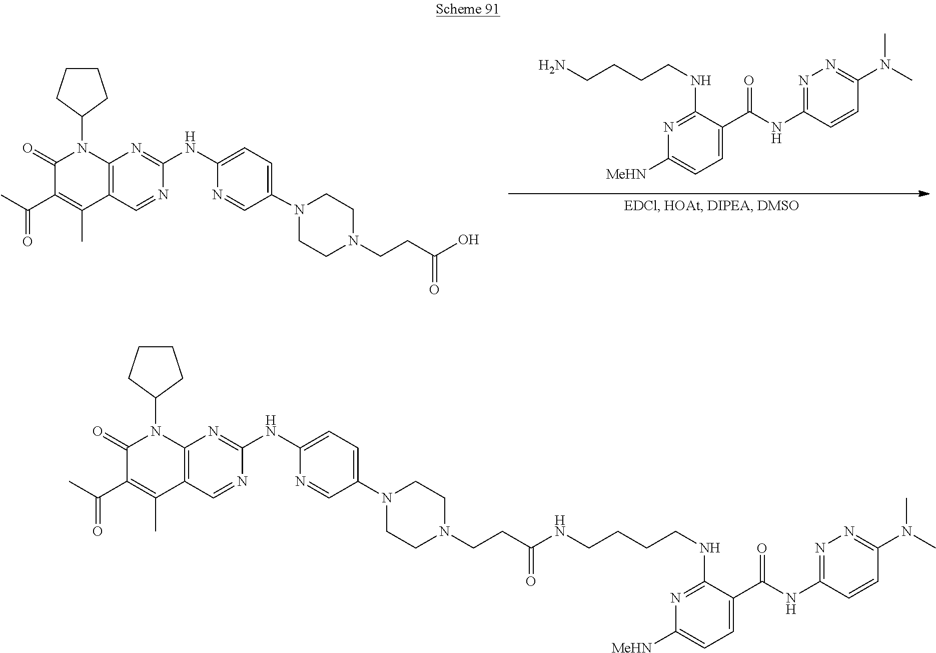 [SMILES: CC(=O)c1c(C)c2cnc(Nc3ccc(N4CCN(CCC(=O)O)CC4)cn3)nc2n(C2CCCC2)c1=O ; CNc1ccc(C(=O)Nc2ccc(N(C)C)nn2)c(NCCCCN)n1 ; CNc1ccc(C(=O)Nc2ccc(N(C)C)nn2)c(NCCCCNC(=O)CCN2CCN(c3ccc(Nc4ncc5c(C)c(C(C)=O)c(=O)n(C6CCCC6)c5n4)nc3)CC2)n1]

CPD-37 was synthesized following the standard procedure for preparing CPD-1 (11 mg, 37% yield) as a yellow solid. MS (ESI) m/z=860.7 [M+H]⁺.

### Example 92. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-methyl-3H-imidazo[4,5-b]pyridin-5-yl)-6-(methylamino)nicotinamide (CPD-38)

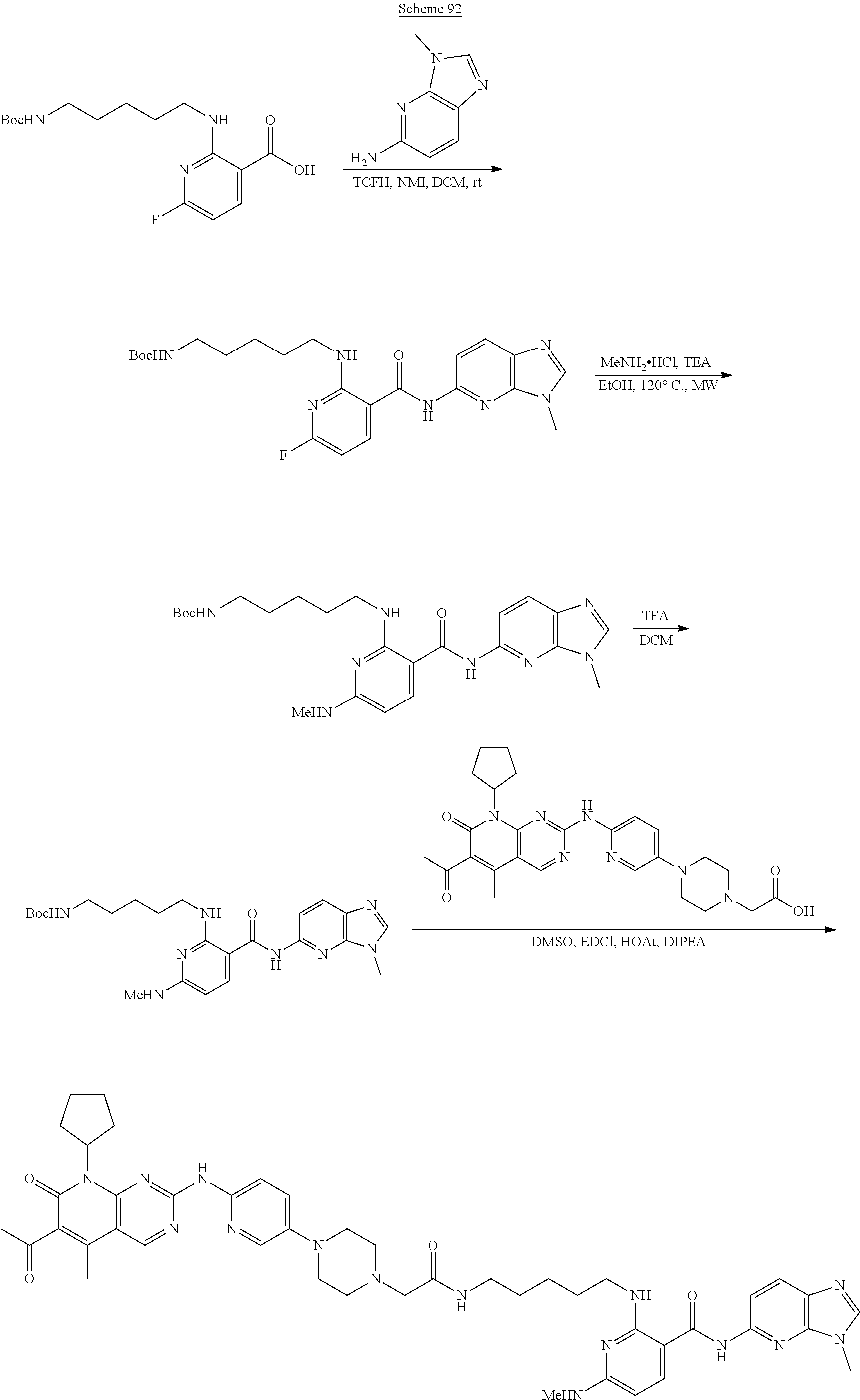

CPD-38 was synthesized following the procedures for preparing CPD-31 (4.9 mg, 5% yield over 4 steps) as a yellow solid. MS (ESI) m/z=870.8 [M+H]⁺.

### Example 93. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-(methylamino)nicotinamide (CPD-39)

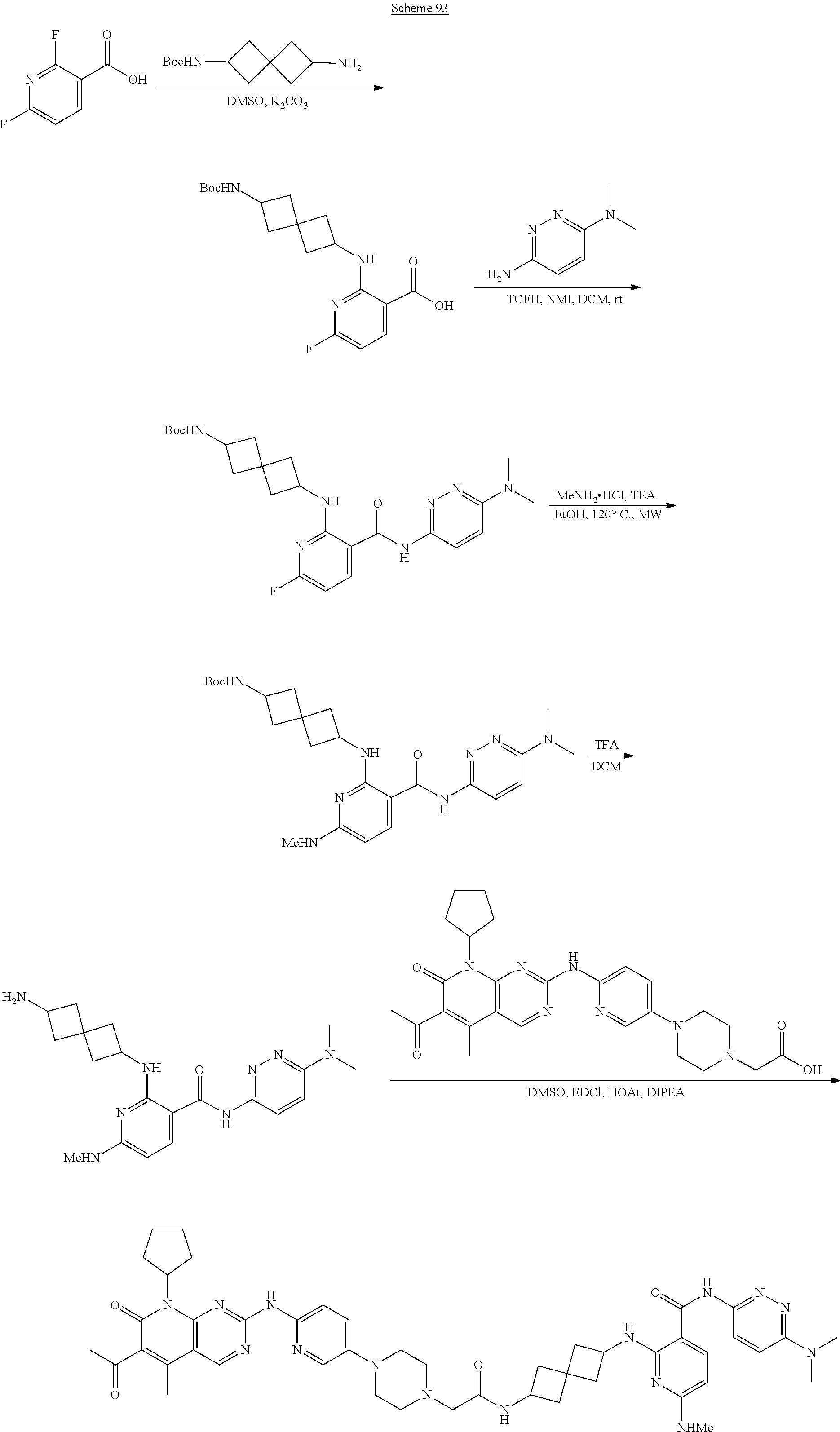

### Step 1. Synthesis of 2-((6-((tert-butoxycarbonyl)amino)spiro[3.3]heptan-2-yl)amino)-6-fluoronicotinic acid

To a solution of 2,6-difluoronicotinic acid (50 mg, 0.3 mmol) and tert-butyl (6-aminospiro[3.3]heptan-2-yl)carbamate (60 mg, 0.26 mmol) in DMSO (5 mL) was added K₂CO₃ (112 mg, 0.81 mmol) at rt. The reaction mixture was stirred at 100° C. for 16 h. After cooling down to rt, the mixture was purified by reverse-phase chromatography to provide the desired product (65 mg, 67% yield) as a white solid. MS (ESI) m/z=366.5 [M+H]⁺.

The remaining steps were performed according to the procedures for preparing CPD-31 to provide the desired product (3.5 mg, 2% yield over 4 steps) as a yellow solid. MS (ESI) m/z=884.8 [M+H]⁺.

### Example 94. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6,7-dihydro-5H-cyclopenta[b]pyridin-2-yl)-6-(methylamino)nicotinamide (CPD-40)

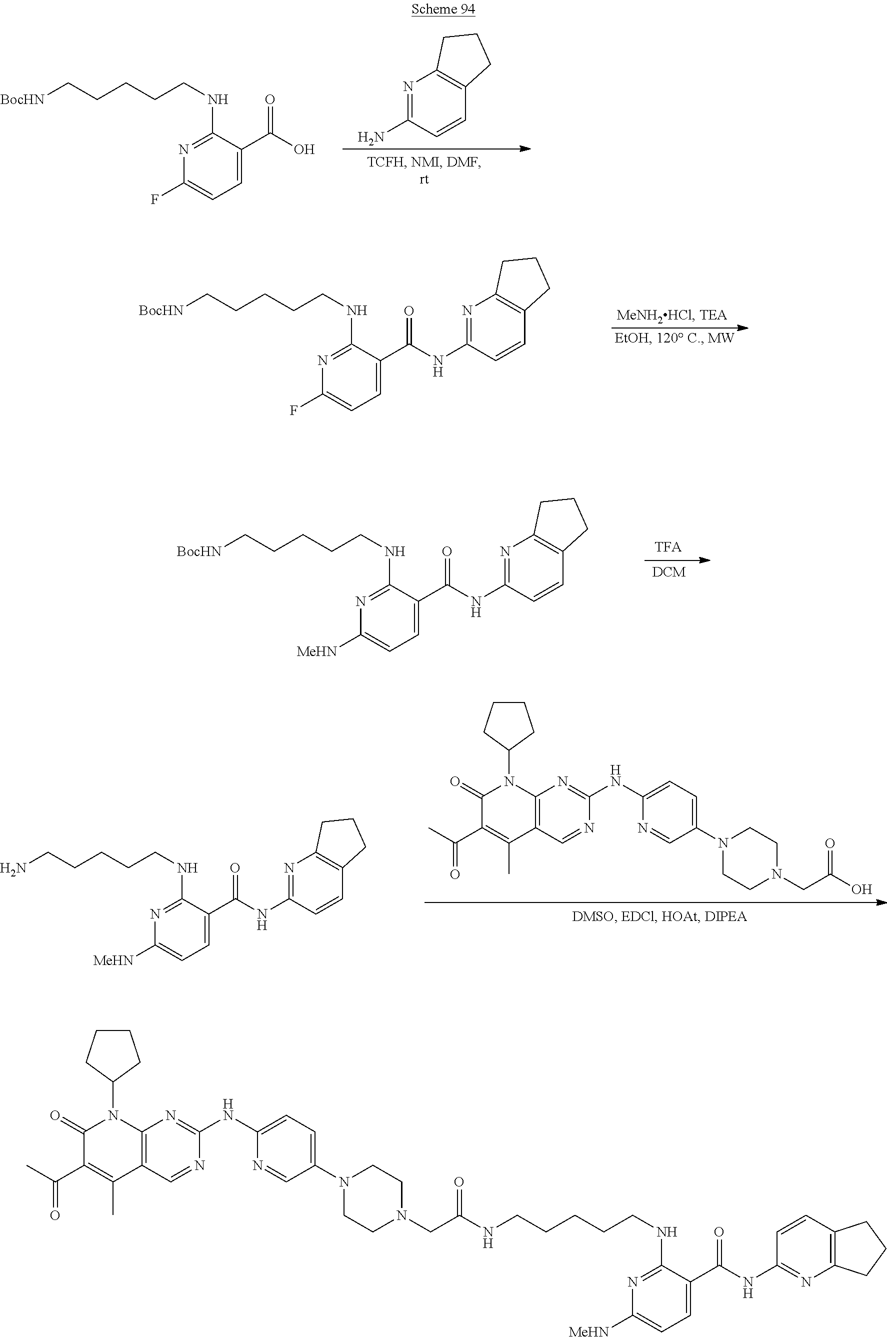

CPD-40 was synthesized following the procedures for preparing CPD-31 (4.6 mg, 1% yield over 4 steps) as a yellow solid. MS (ESI) m/z=856.8 [M+H]⁺.

### Example 95. 2-((2-(3-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)ethyl)amino)-N-(6-(dimethylamino)pyridazin-3-yl)-6-(methylamino)nicotinamide (CPD-41)

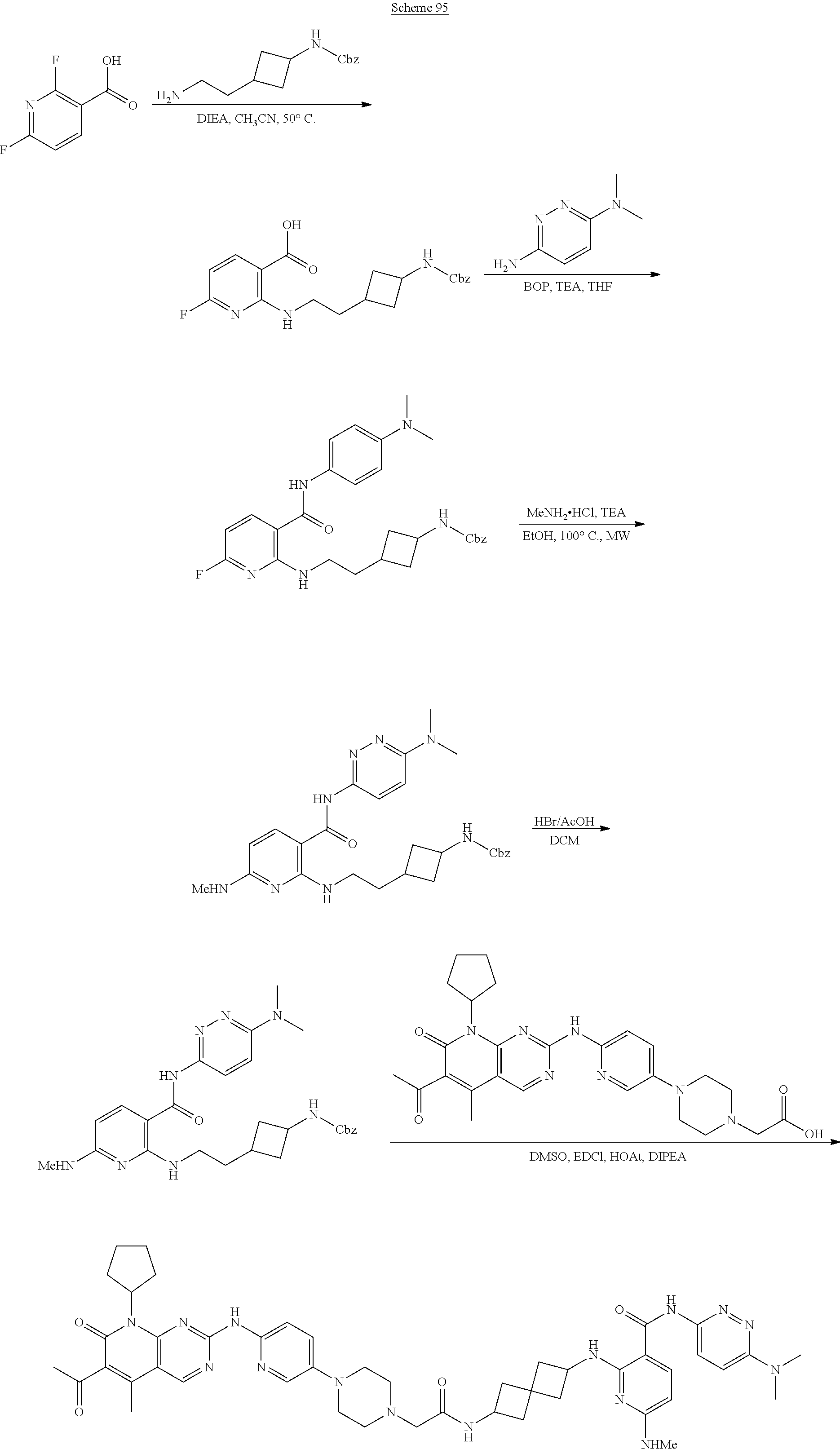

### Step 1. Synthesis of 2-[2-[3-(benzyloxycarbonylamino)cyclobutyl]ethylamino]-6-fluoro-pyridine-3-carboxylic acid

The title compound was synthesized following the procedure of step 3 for preparing CPD-34 (250 mg, 21%0 yield) as a yellow solid. ¹HNMR (400 MHz, MeOH-d₄) δ 8.24 (t, J=8.4 Hz, 1H), 7.35-7.28 (m, 5H), 6.10 (dd, J=2.8, 8.4 Hz, 1H), 5.06-5.03 (m, 2H), 4.19-3.90 (m, 1H), 3.44-3.36 (m, 2H), 2.48-2.44 (M, 1H), 2.13-1.77 (m, 4H), 1.73-1.69 (m, 2H), 1.62-1.59 (in, H). MS (ESI) m/z=388.4 [M+H]⁺.

### Step 2. Synthesis of benzyl N-[3-[2-[[3-[[6-(dimethylamino)pyridazin-3-yl]carbamoyl]-6-fluoro-2-pyridyl]amino]ethyl]cyclobutyl]carbamate

The title compound was synthesized following the procedure of step 4 for preparing CPD-25 (180 mg, 57% yield) as a yellow solid. ¹HNMR (400 MHz, CDCl₃) δ 8.46 (s, 1H), 8.1 (t, J=8.0 Hz, 1H), 7.37-7.33 (m, 5H), 7.24 (d, J=9.2 Hz, 1H), 6.98 (d, J=10.0 Hz, 1H), 6.11 (dd, J=2.8, 8.4 Hz, 1H), 5.09-5.07 (m, 2H), 4.95-4.84 (m, 2H), 4.37-3.99 (m, 1H), 3.45-3.39 (m, 2H), 3.18 (s, 6H), 2.55-2.49 (m, 1H), 2.16-2.11 (m, 2H), 1.84-1.81 (m, 2H), 1.76-1.72 (m, 1H). MS (ESI) z/z=508.4 [M+H]⁺.

### Step 3. Synthesis of benzyl N-[3-[2-[[3-[[6-(dimethylamino)pyridazin-3-yl]carbamoyl]-6-(methylamino)-2-pyridyl]amino]ethyl]cyclobutyl]carbamate

The title compound was synthesized following the procedure of step 5 for preparing CPD-25 (0.14 g, crude) as a yellow oil. ¹HNMR (400 MHz, MeOH-d) δ 8.07-7.92 (m, 1H), 7.76 (d, J=8.4 Hz, 1H), 7.37-7.25 (i, 5H), 7.19 (d, J=10.0 Hz, 1H), 5.74 (d, J=8.8 Hz, 1H), 5.04 (d, J=4.0 Hz, 2H), 4.27-3.85 (m, 1H), 3.50-3.40 (m, 2H), 3.10 (s, 6H), 2.91 (s, 3H), 2.52-2.25 (m, 2H), 2.14-2.08 (m, 2H), 1.81-1.49 (m, 5H). MS (ESI) m/z=519.5 [M+H]⁺.

### Step 4. Synthesis of 2-[2-(3-aminocyclobutyl)ethylamino]-N-[6-(dimethylamino)pyridazin-3-yl]-6-(methylamino)pyridine-3-carboxamide

To a solution of benzyl N-[3-[2-[[3-[[6-(dimethylamino)pyridazin-3-yl]carbamoyl]-6-(methylamino)-2-pyridyl]amino]ethyl]cyclobutyl]carbamate (130 mg, 0.25 mmol) in DCM (2 mL) was added HBr (33 wt % in AcOH, 1.49 g, 6.08 mmol) at rt. The reaction mixture was stirred at rt for 0.5 h, before it was concentrated and purified by prep-HPLC to provide the desired product (15 mg, 14% yield) as a yellow solid. ¹HNMR (400 MHz, MeOH-d₄) b 7.98 (d, J=10.0 Hz, 1H), 7.77 (d, J=8.4 Hz, 1H), 7.18 (d, J=9.6 Hz, 1H), 5.75 (d, J=8.0 Hz, 1H), 3.63-3.56 (m, 0.5H), 3.52-3.43 (m, 2.5H), 3.14 (s, 6H), 2.92 (s, 3H), 2.49-2.38 (m, 1.5H), 2.14-2.00 (m, 2.5H), 1.83-1.72 (m, 2H), 1.57-1.49 (m, 1H). MS (ESI) m/z=385.2 [M+H]⁺.

### Step 5. Synthesis of 2-((2-(3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)ethyl)amino)-N-(6-(dimethylamino)pyridazin-3-yl)-6-(methylamino)nicotinamide

The title compound was synthesized following the procedure for preparing CPD-1 (12.8 mg, 49% yield) as a yellow solid. MS (ESI) m/z=872.8 [M+H]⁺.

### Example 96. 2-(4-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)piperidin-1-yl)-N-(6-(dimethylamino)pyridazin-3-yl)-6-(methylamino)nicotinamide (CPD-42)

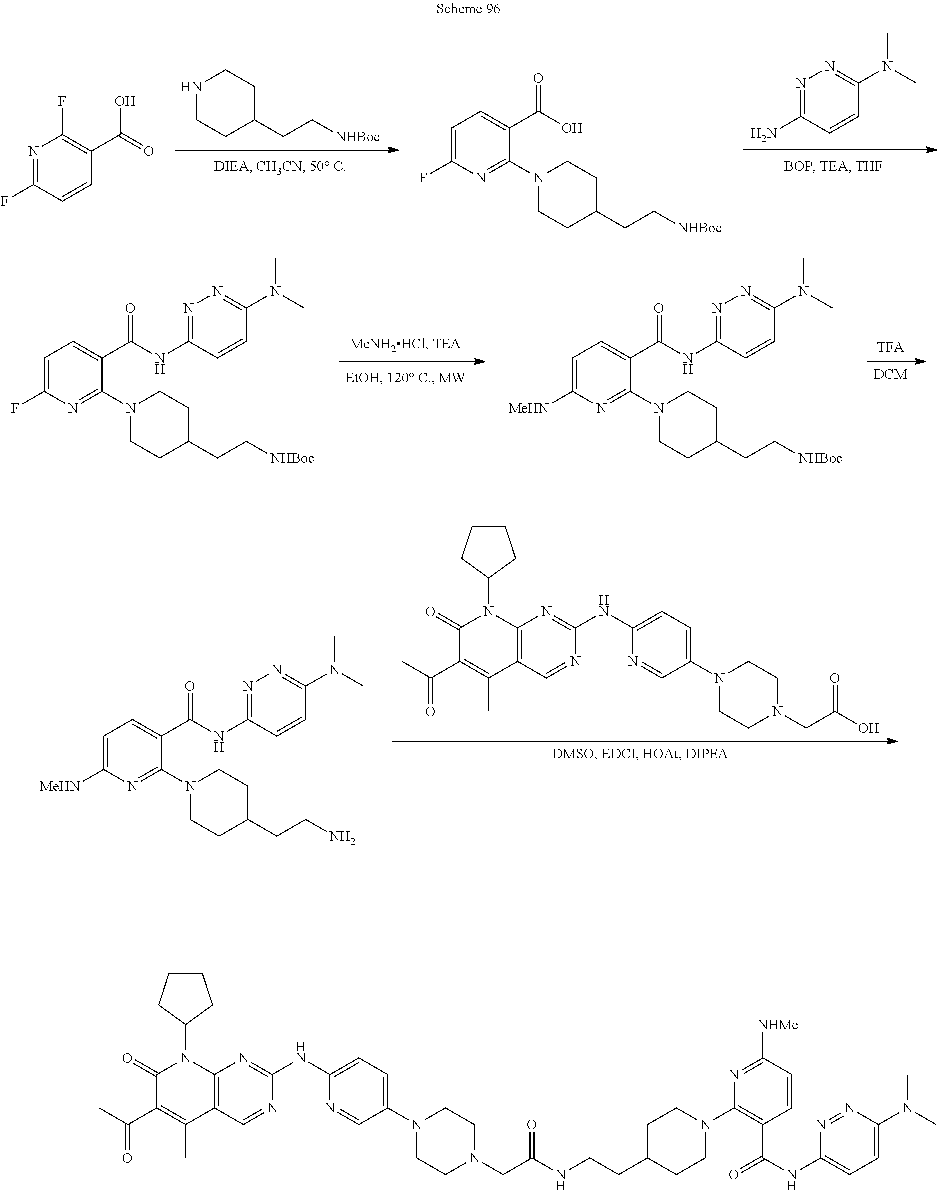

CPD-42 was synthesized following the procedures for preparing CPD-34 (5.9 mg, 1% yield over 5 steps) as a yellow solid. MS (ESI) m/z=886.8 [M+H]⁺.

### Example 97. 2-((trans-4-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclohexyl)amino)-N-(6-(dimethylamino)pyridazin-3-yl)-6-(methylamino)nicotinamide (CPD-43)

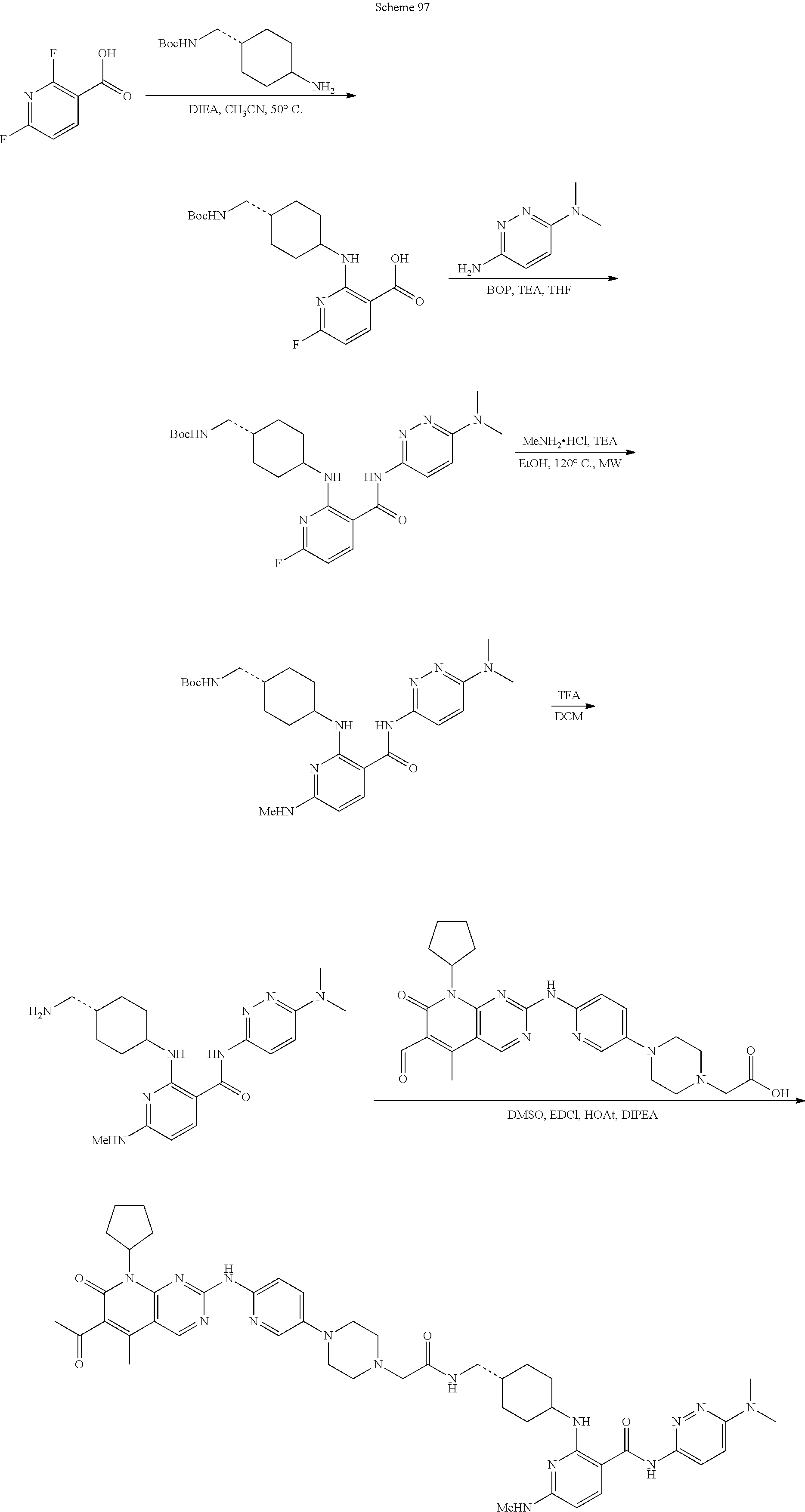

CPD-43 was synthesized following the procedures for preparing CPD-34 (7.7 mg, 1% yield over 5 steps) as a yellow solid. MS (ESI) m/z=886.9 [M+H]⁺.

### Example 98. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-cyclopropyl-N-(6-cyclopropyl-5-methylpyridin-2-yl)nicotinamide (CPD-44)

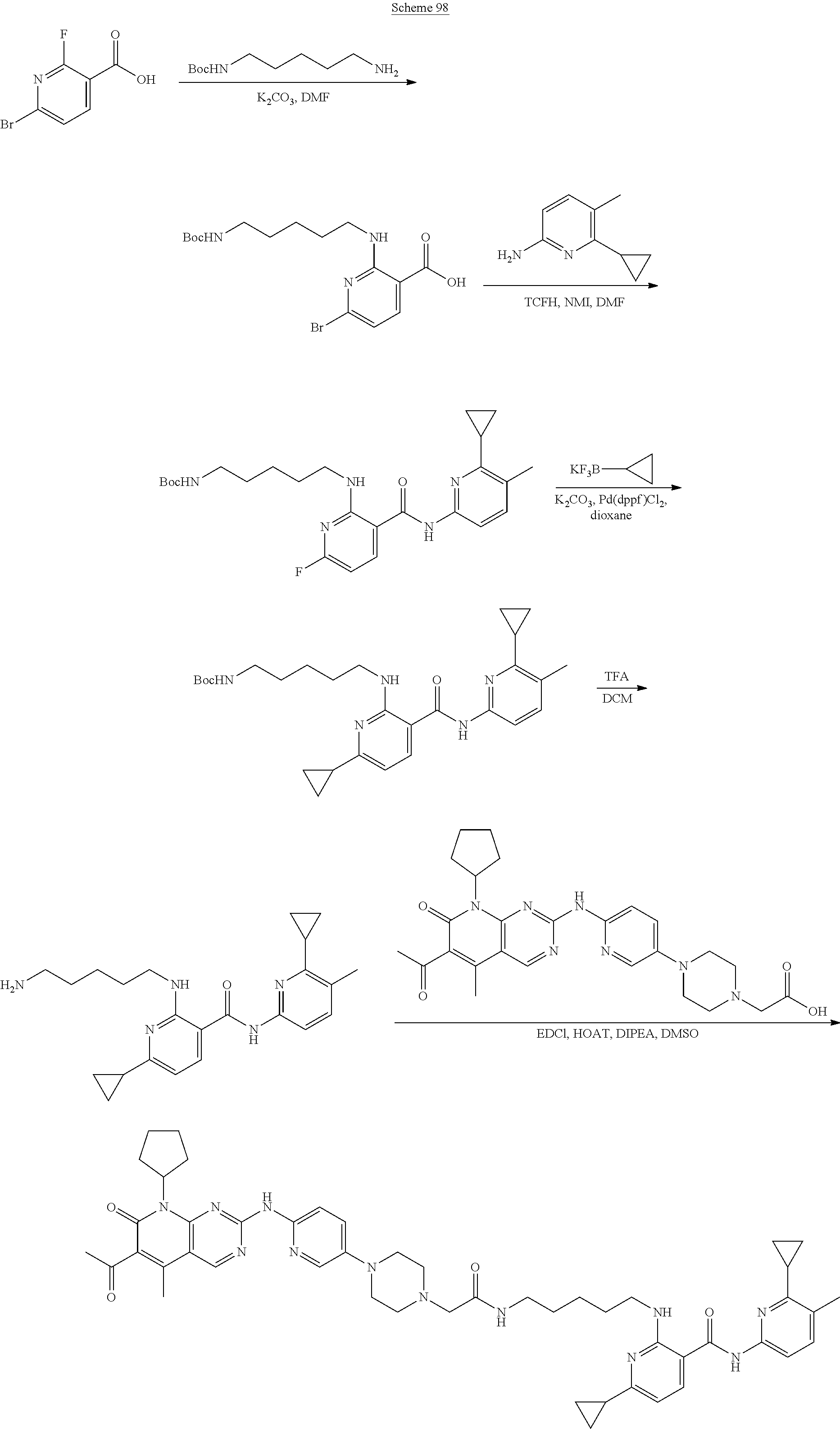

### Step 1. Synthesis of 6-bromo-2-((5-((tert-butoxycarbonyl)amino)pentyl)amino)nicotinic acid

To a solution of 6-bromo-2-fluoronicotinic acid (300 mg, 1.36 mmol) in DMF (10 mL) were added tert-butyl (5-aminopentyl)carbamate (276 mg, 1.36 mmol) and K₂CO₃ (940 mg, 6.82 mmol). The reaction mixture was stirred at 80° C. for 3 h. After cooling down to rt, the solution was diluted with water (50 mL) and the pH was adjusted to 5-6 with hydrochloric acid solution. The aqueous phase was extracted with EtOAc (3×60 mL). The combined organic layers were washed with saturated brine (20 mL), dried over anhydrous sodium sulfate, filtered, and evaporated under reduced pressure. The residue was purified by reverse-phase chromatography to provide the desired product (500 mg, 91% yield) as a light-yellow solid. MS (ESI) m/z=402.4 [M+H]⁺.

### Step 2. Synthesis of tert-butyl (5-((6-bromo-3-((6-cyclopropyl-5-methylpyridin-2-yl)carbamoyl)pyridin-2-yl)amino)pentyl)carbamate

To a solution of 6-bromo-2-((5-((tert-butoxycarbonyl)amino)pentyl)amino)nicotinic acid (100 mg, 0.25 mmol) in DMF (5 mL) were added 6-cyclopropyl-5-methylpyridin-2-amine (37 mg, 0.25 mmol), NMI (105 mg, 1.24 mmol) and TCFH (90 mg, 0.32 mmol). The mixture was stirred at rt for 2 h. The reaction mixture was poured into water (30 mL) and extracted with ethyl acetate (3×30 mL). The combined organic layers were washed with saturated brine (20 mL), dried over anhydrous sodium sulfate, filtered, and evaporated under reduced pressure. The residue was purified by silica gel chromatography to provide the desired product (100 mg, 76% yield) as a light-yellow solid. MS (ESI) m/z=532.5 [M+H]⁺.

### Step 3. Synthesis of tert-butyl (5-((6-cyclopropyl-3-((6-cyclopropyl-5-methylpyridin-2-yl)carbamoyl)pyridin-2-yl)amino)pentyl)carbamate

To a solution of tert-butyl (5-((6-bromo-3-((6-cyclopropyl-5-methylpyridin-2-yl)carbamoyl)pyridin-2-yl)amino)pentyl)carbamate (70 mg, 0.13 mmol) in dioxane (10 mL) were added potassium cyclopropyltrifluoroborate (40 mg, 0.26 mmol), Pd(dppf)Cl₂ (10 mg, 0.013 mmol) and K₂CO₃ (91 mg, 0.66 mmol) at rt. The reaction mixture was stirred at 100° C. under argon atmosphere for 6 h. After cooling down to rt, the reaction mixture was poured into water (30 mL) and extracted with ethyl acetate (3×30 mL). The combined organic layers were washed with saturated brine (20 mL), dried over anhydrous sodium sulfate, filtered, and evaporated under reduced pressure. The residue was purified by silica gel chromatography to provide the desired product (15 mg, 23% yield) as a brown solid. MS (ESI) m/z=494.6 [M+H]⁺.

The remaining steps was performed according to the standard procedures to provide the desired product (3.0 mg, 13% yield over 2 steps) as a yellow solid. MS (ESI) m/z=881.8 [M+H]⁺.

### Example 99. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-cyclopropyl-N-(6-cyclopropyl-5-methylpyridin-2-yl)nicotinamide (CPD-45)

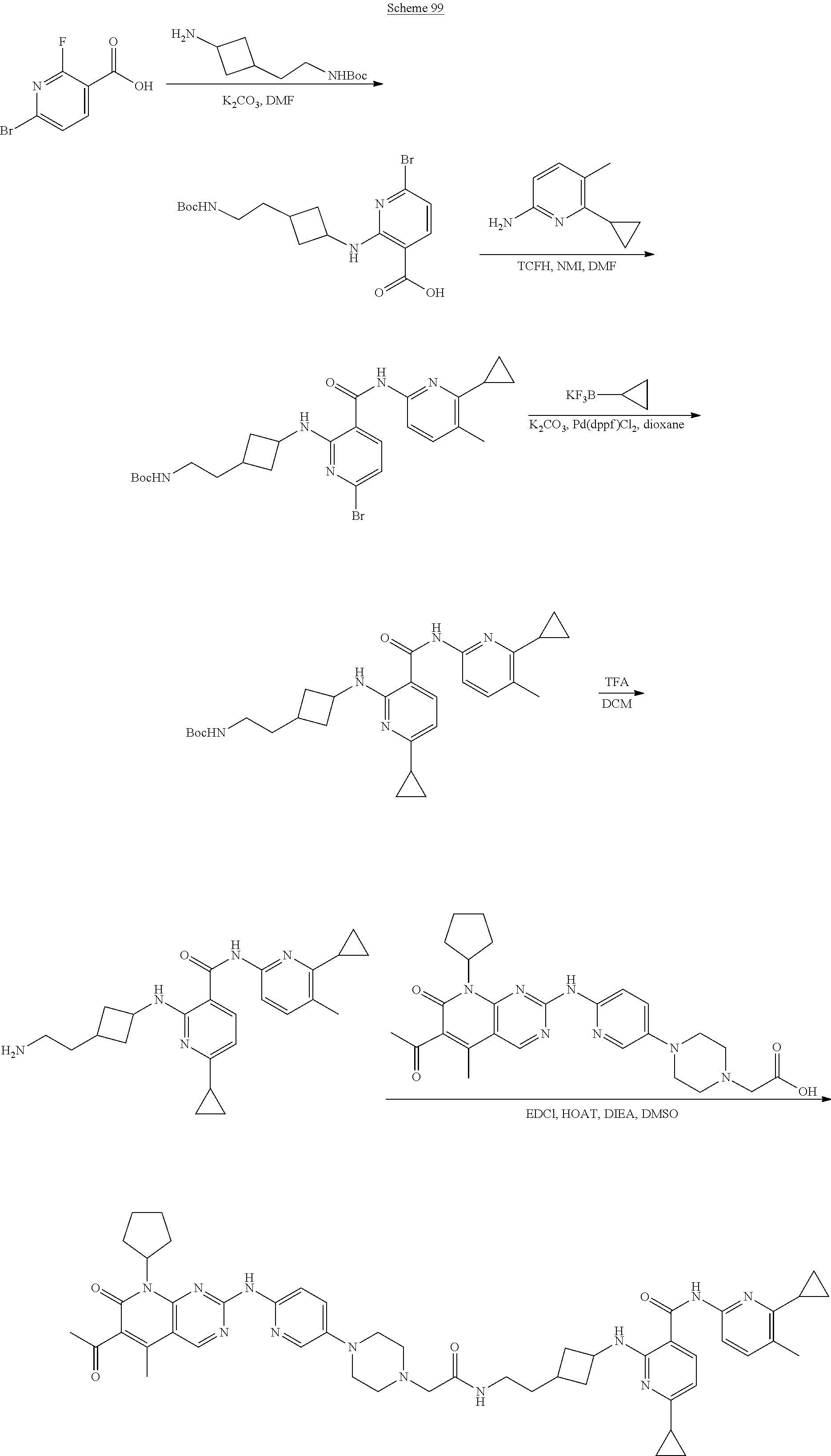

CPD-45 was synthesized following the procedures for preparing CPD-44 (5.6 mg, 3% yield over 5 steps) as a yellow solid. MS (ESI) m/z=893.8 [M+H]⁺.

### Example 100. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-(dimethylamino)phenyl)-6-(methylamino)nicotinamide (CPD-46)

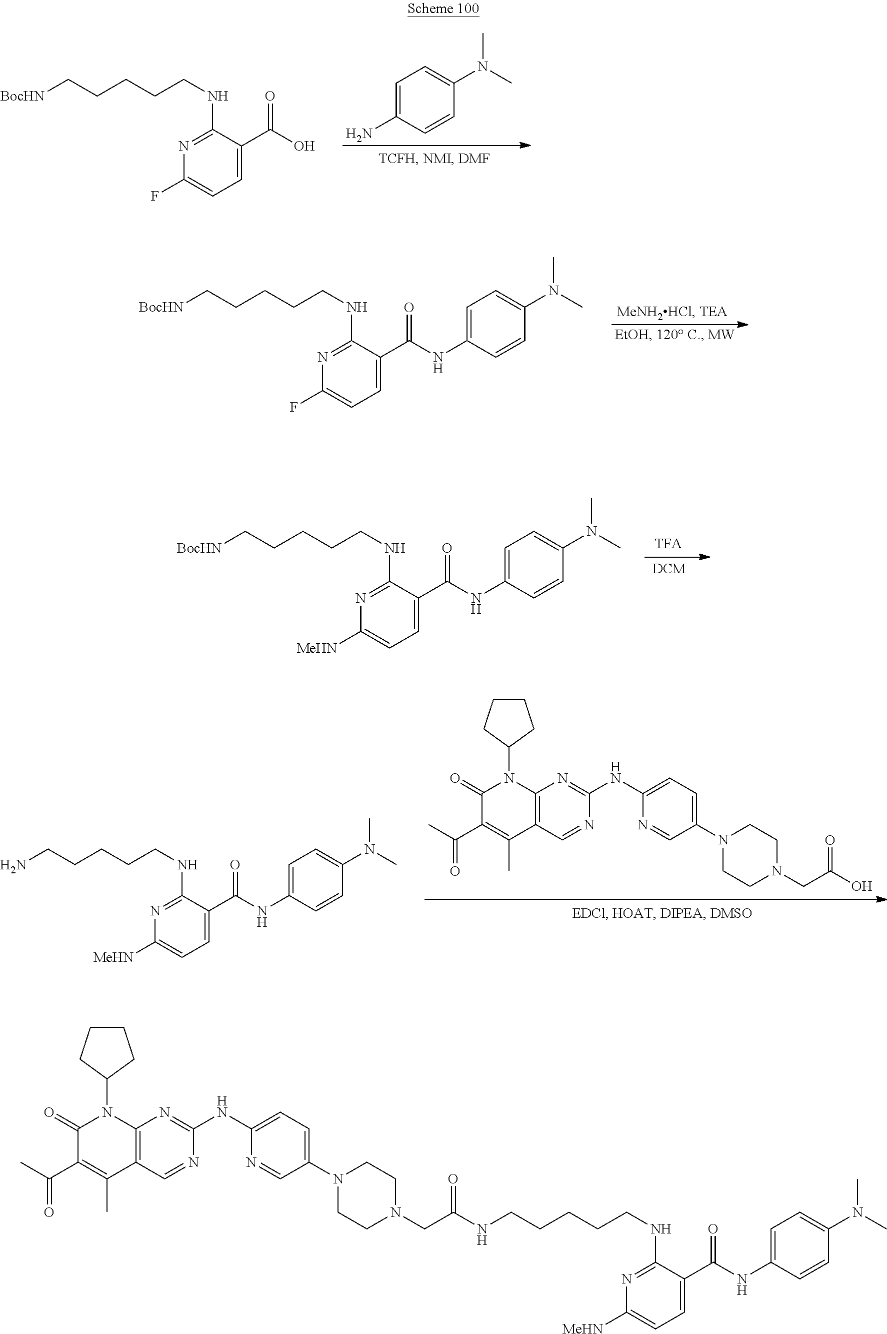

CPD-46 was synthesized following the procedures for preparing CPD-31 (8 mg, 15% yield over 4 steps) as a yellow solid. MS (ESI) m/z=858.8 [M+H]⁺.

### Example 101. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-(dimethylamino)pyridin-2-yl)-6-(methylamino)nicotinamide (CPD-47)

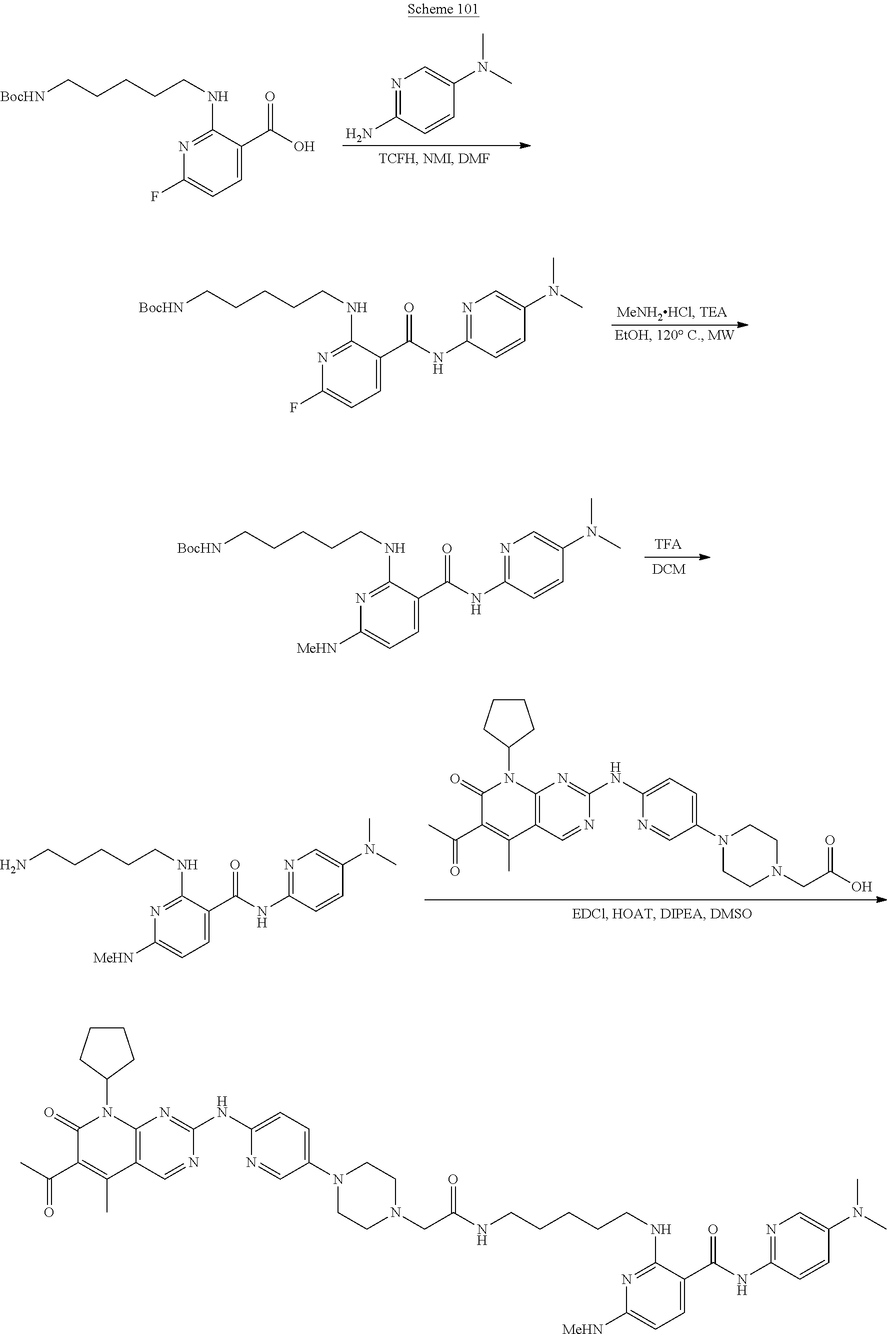

CPD-47 was synthesized following the procedures for preparing CPD-31 (9 mg, 26% yield over 4 steps) as a yellow solid. MS (ESI) m/z=859.9 [M+H]⁺.

### Example 102. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-(dimethylamino)cyclohexyl)-6-(methylamino)nicotinamide (CPD-48)

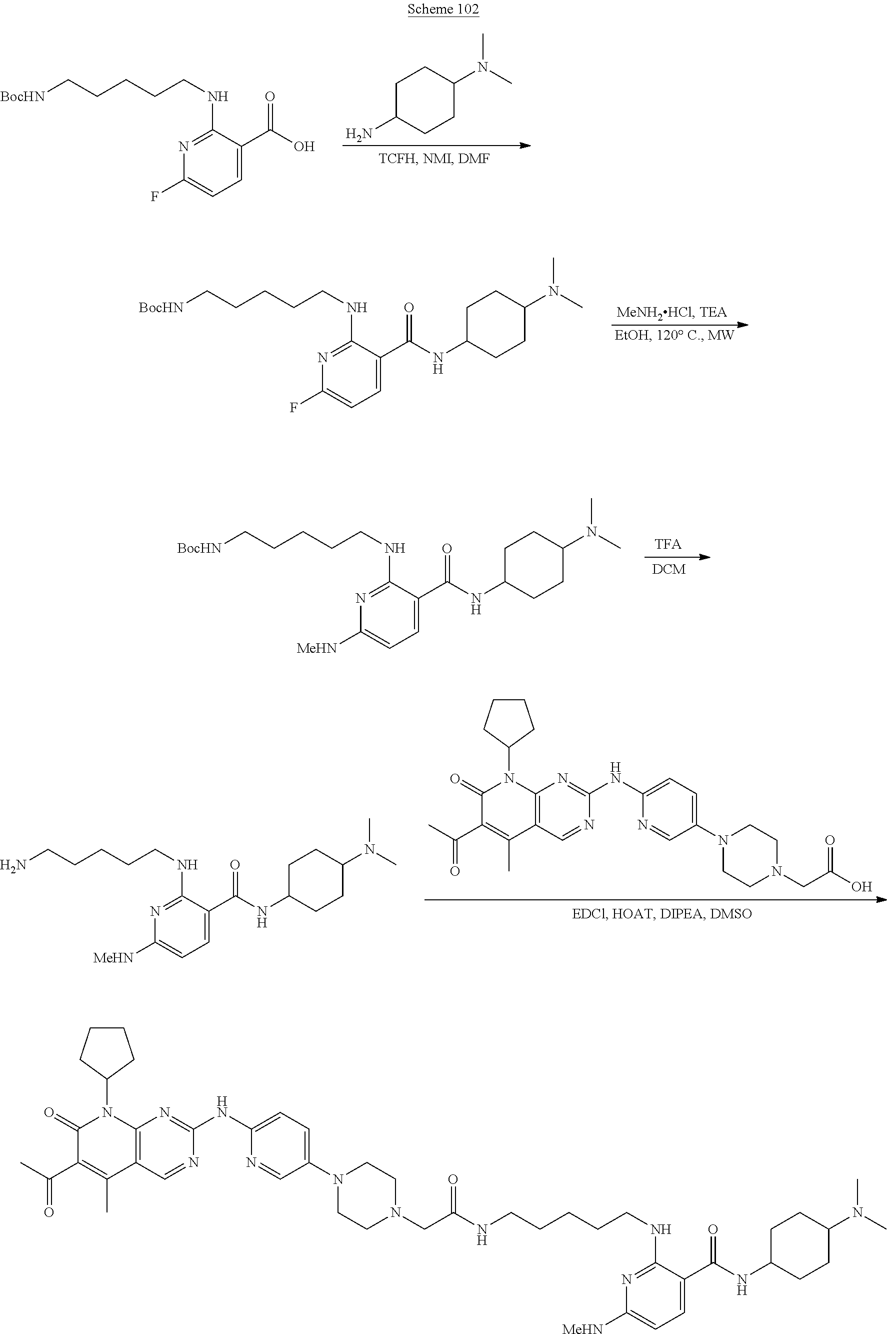

CPD-48 was synthesized following the procedures for preparing CPD-31 (10 mg, 22% yield over 4 steps) as a yellow solid. MS (ESI) m/z=864.9 [M+H]⁺.

### Example 103. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1-methyl-1H-pyrazol-3-yl)-6-(methylamino)nicotinamide (CPD-49)

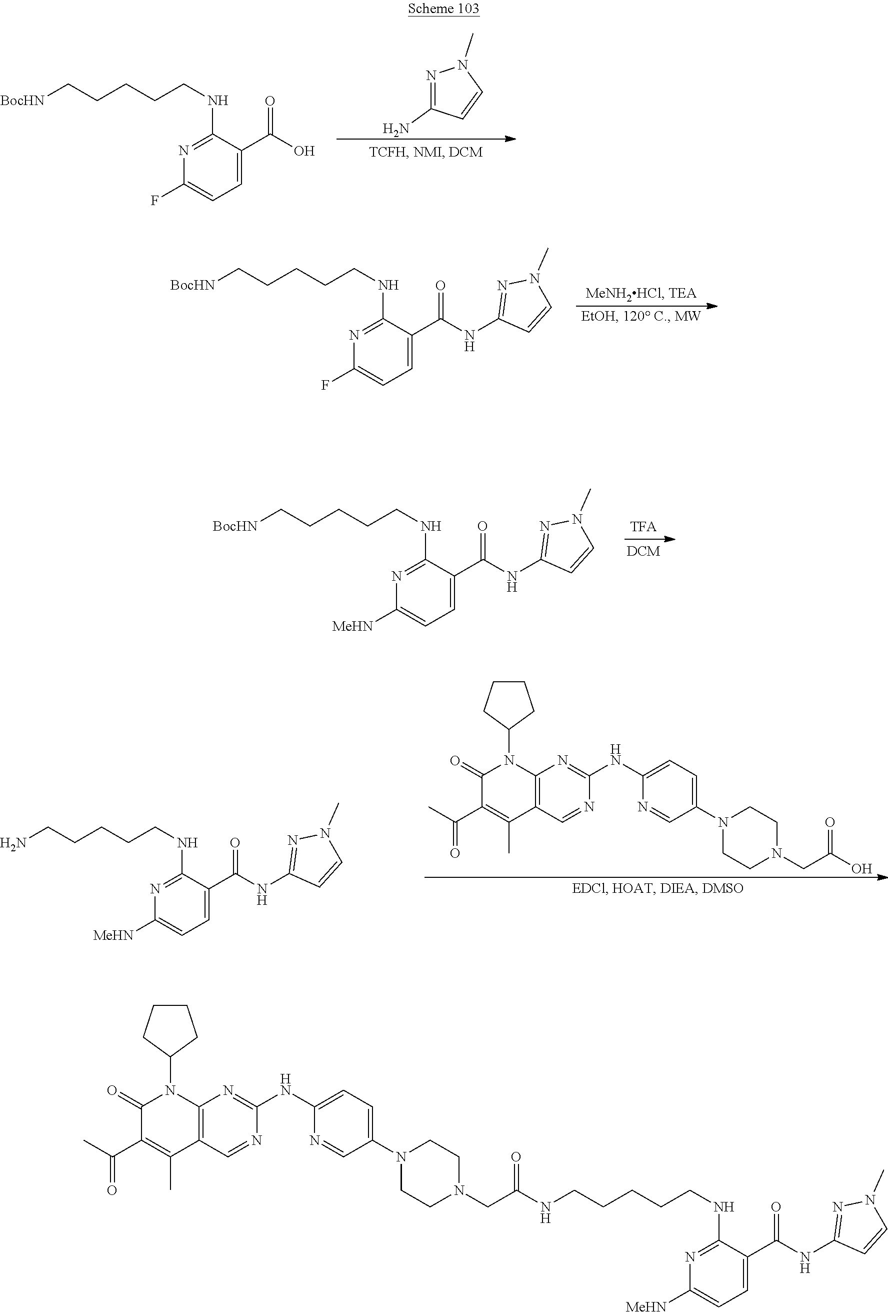

CPD-49 was synthesized following the procedures for preparing CPD-31 (3.4 mg, 3% yield over 4 steps) as a yellow solid. MS (ESI) m/z=819.7 [M+H]⁺.

### Example 104. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(1,5-dimethyl-1H-pyrazol-3-yl)-6-(methylamino)nicotinamide (CPD-50)

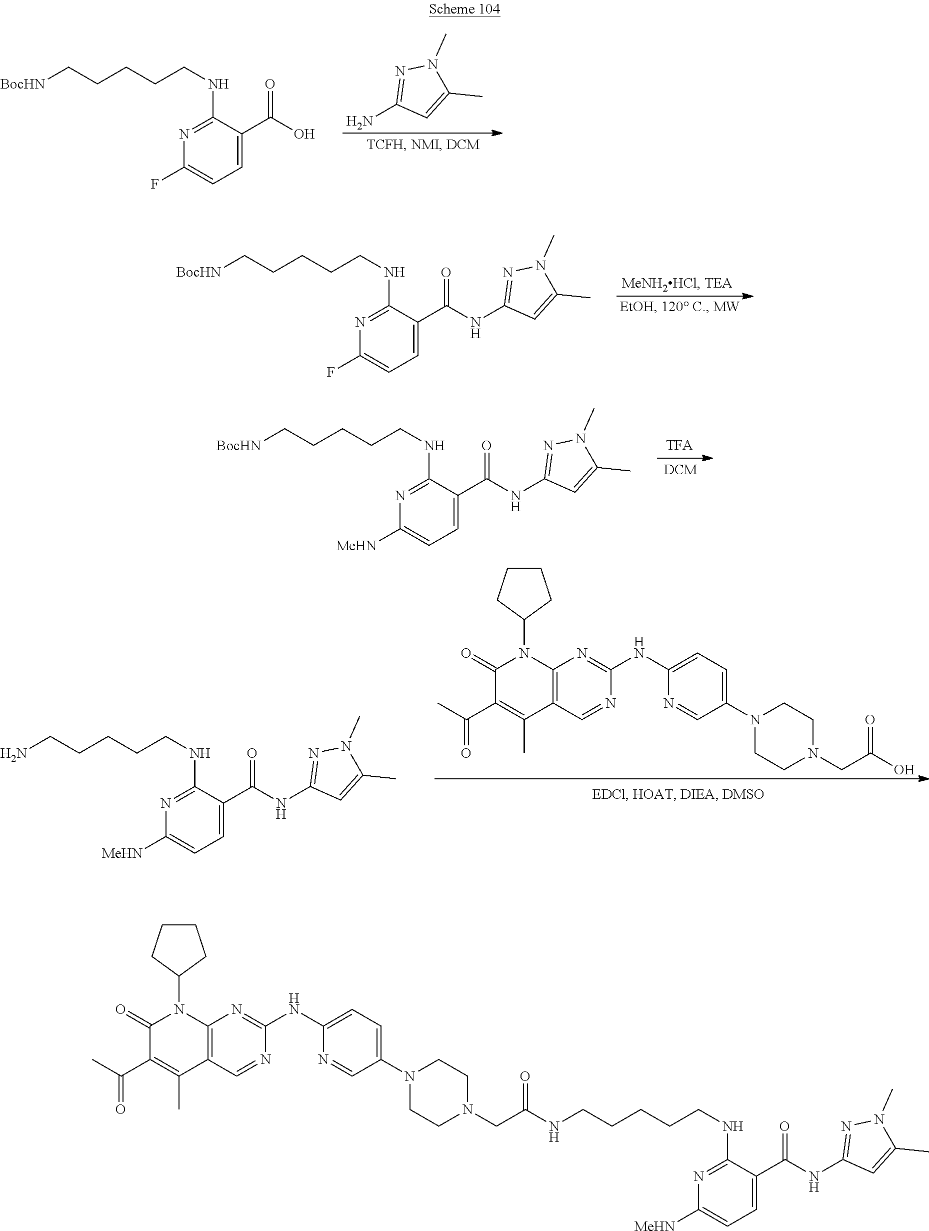

CPD-50 was synthesized following the procedures for preparing CPD-31 (5.8 mg, 5% yield over 4 steps) as a yellow solid. MS (ESI) m/z=833.7 [M+H]⁺.

### Example 105. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridin-2-yl)-6-(methylamino)nicotinamide (CPD-51)

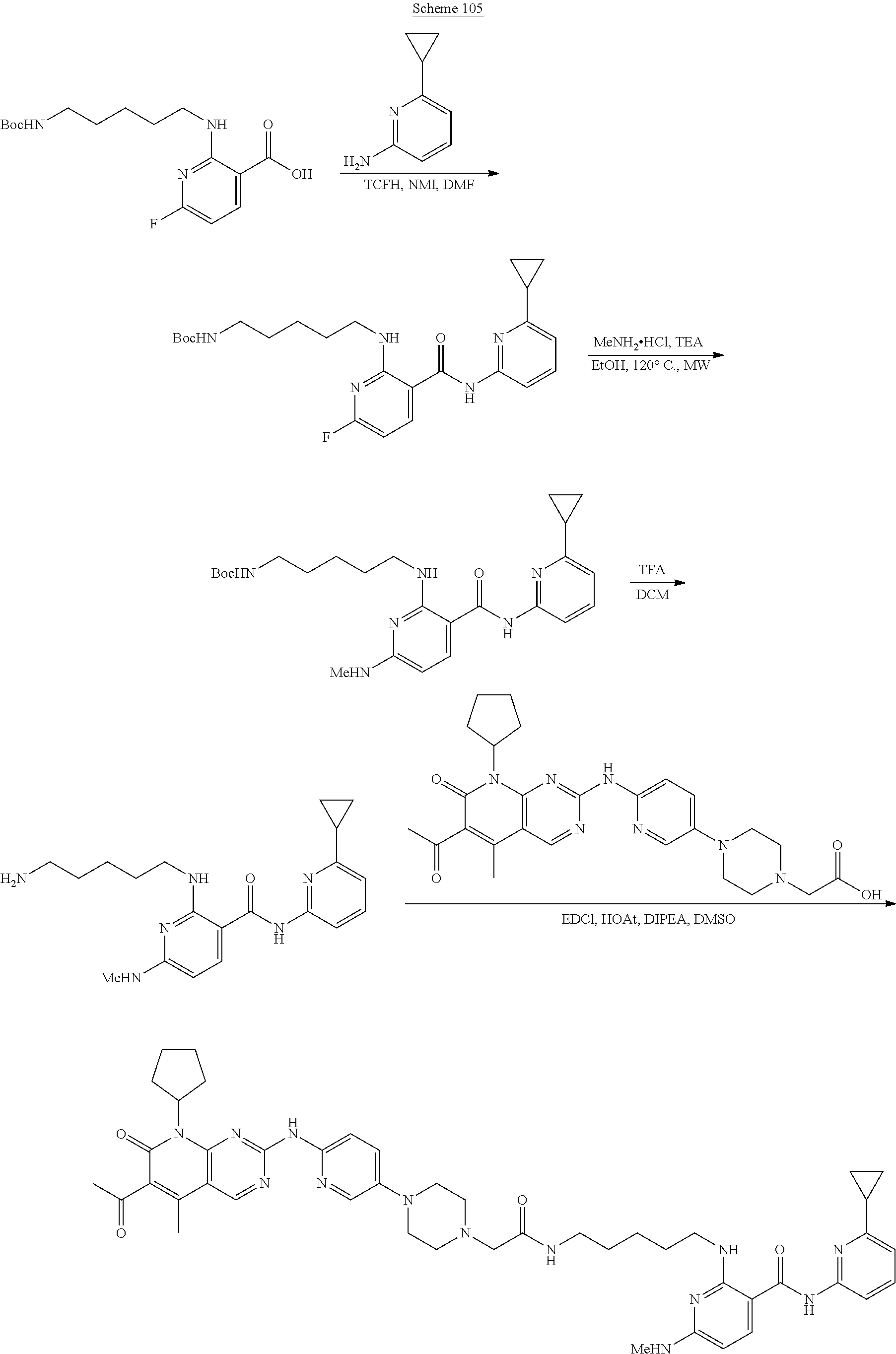

CPD-51 was synthesized following the procedures for preparing CPD-31 (5.2 mg, 1% yield over 4 steps) as a yellow solid. MS (ESI) m/z=856.9 [M+H]⁺.

### Example 106. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropylpyridin-2-yl)-6-(methylamino)nicotinamide (CPD-52)

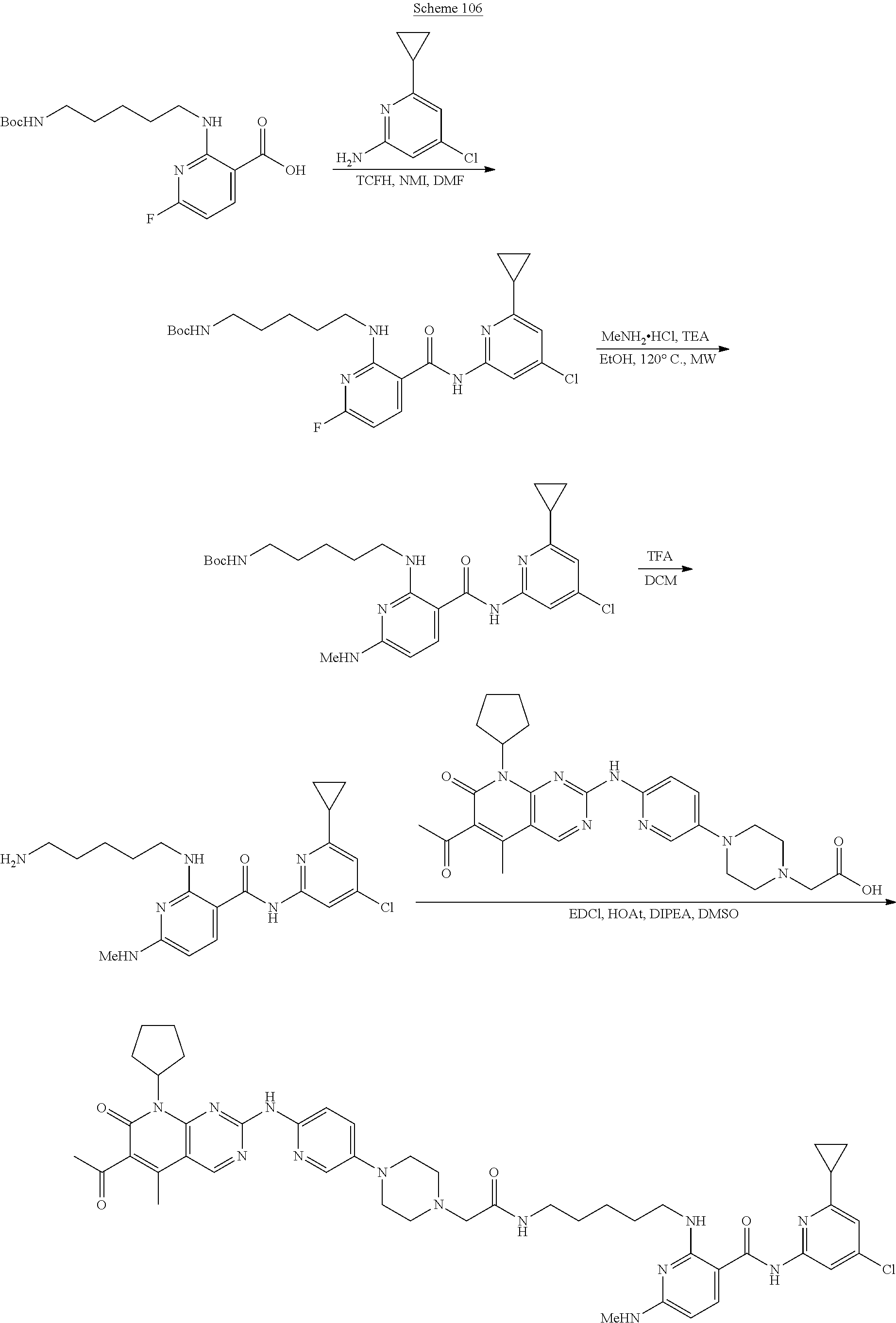

CPD-52 was synthesized following the procedures for preparing CPD-31 (1.5 mg, 1% yield over 4 steps) as a yellow solid. MS (ESI) m/z=890.8 [M+H]⁺.

### Example 107. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-4-methylpyridin-2-yl)-6-(methylamino)nicotinamide (CPD-53)

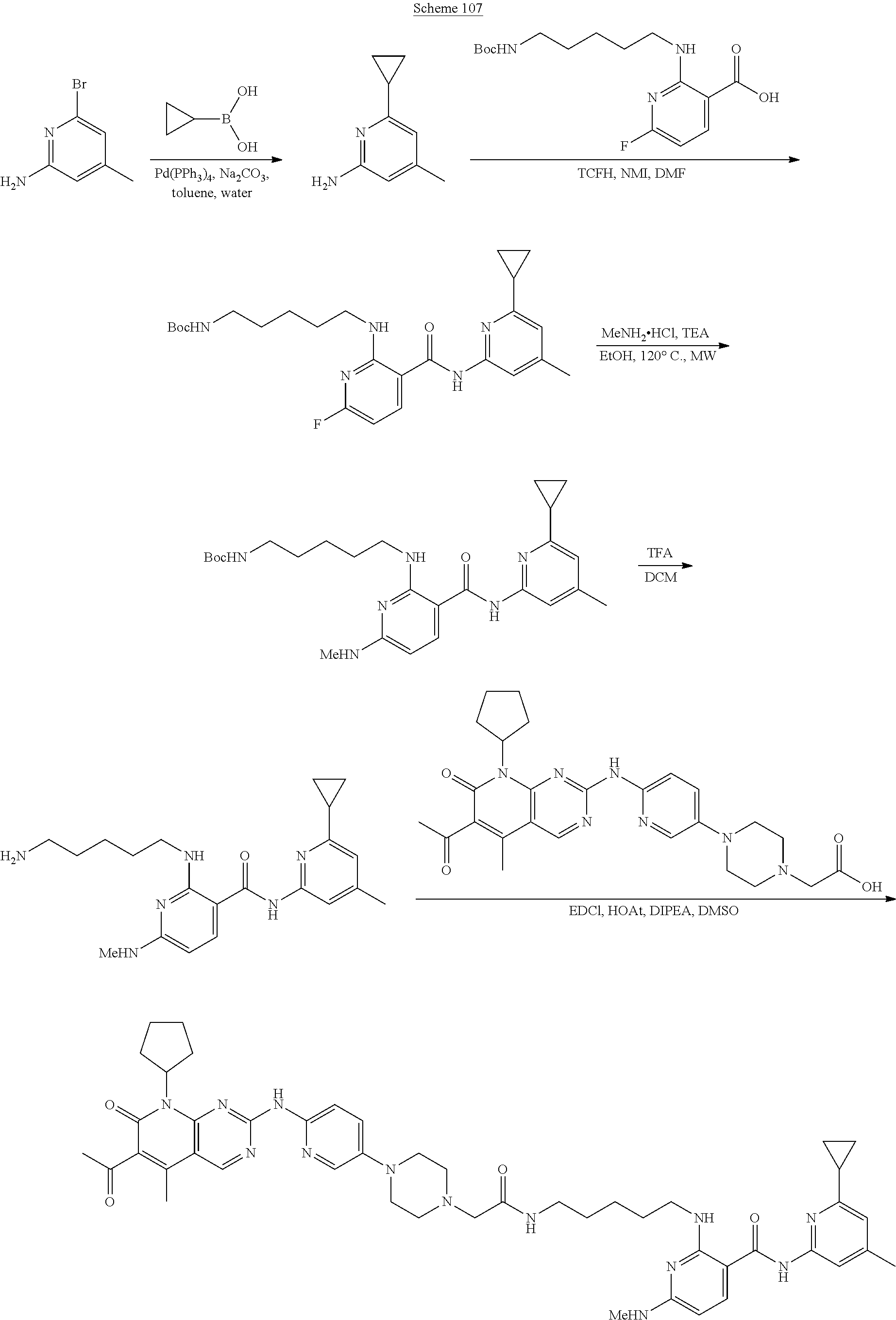

### Step 1. Synthesis of 6-cyclopropyl-4-methylpyridin-2-amine

To a solution of 6-bromo-4-methyl-pyridin-2-amine (1.08 g, 5.78 mmol) in toluene (15 mL) and water (15 mL) were added Na₂CO₃ (612.6 mg, 5.78 mmol) and Pd(PPh₃)₄ (667.6 mg, 0.578 mmol). The reaction mixture was stirred at 80° C. for 2 h. After cooling down to rt, the reaction was diluted with water (15 mL) and extracted with ethyl acetate (20 mL×3). The organic phase was washed with brine, dried over anhydrous Na₂SO₄, filtered, and concentrated. The residue was purified by reverse-phase chromatography to provide the desired product (500 mg, 58% yield) as a light yellow solid. MS (ESI) m/z=149.3 [M+H]⁺.

The remaining steps were performed according to the procedures for preparing CPD-31 to provide the desired product (3.1 mg, 12% yield over 4 steps) as a yellow solid. MS (ESI) m/z=870.8 [M+H]⁺.

### Example 108. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-ethylnicotinamide (CPD-54)

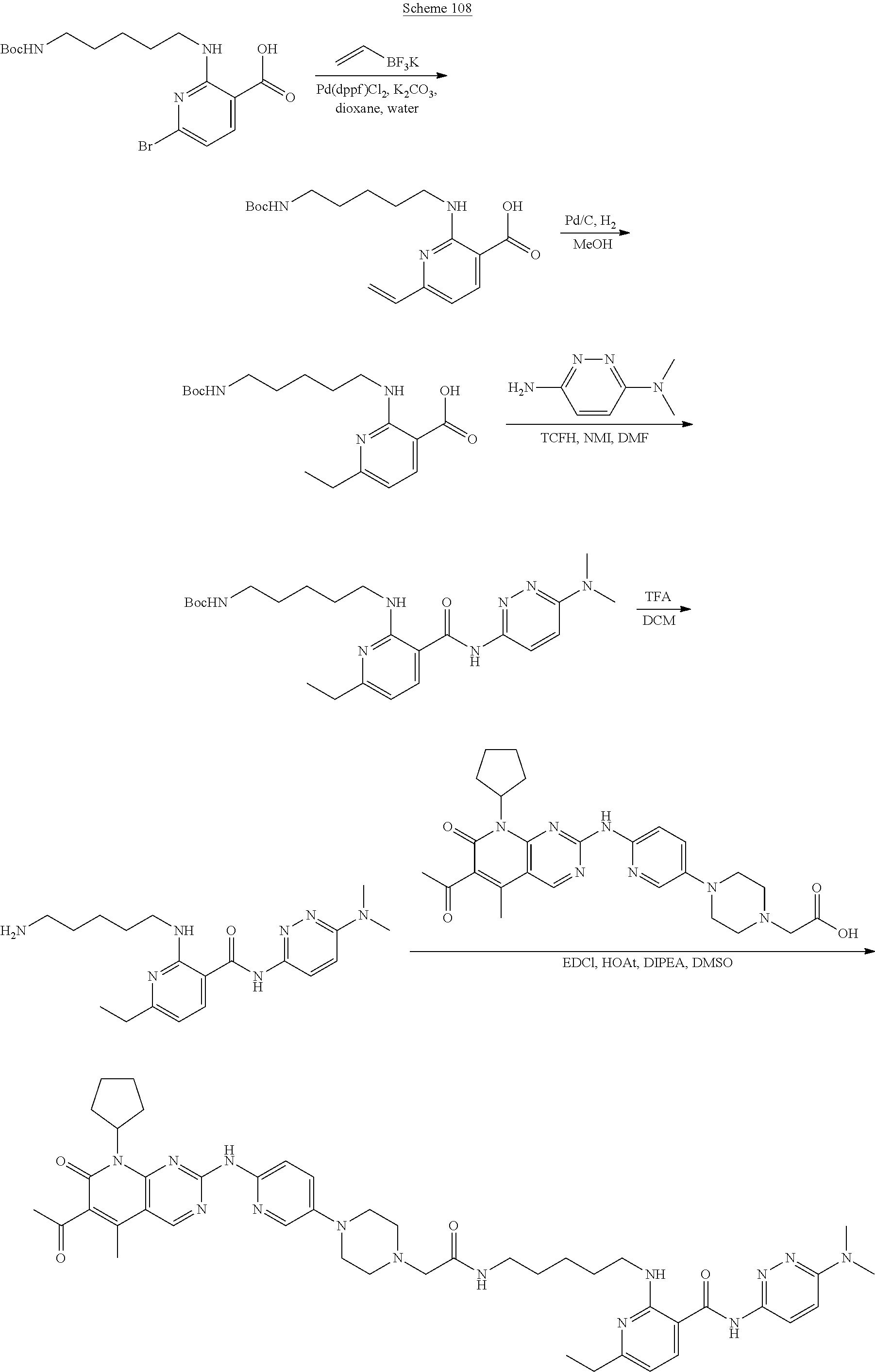

### Step 1. Synthesis of 2-((5-((tert-butoxycarbonyl)amino)pentyl)amino)-6-vinylnicotinic acid

To a solution of 6-bromo-2-((5-((tert-butoxycarbonyl)amino)pentyl)amino)nicotinic acid (50 mg, 0.12 mmol) and potassium vinyltrifluoroborate (11.8 mg, 0.12 mmol) in dioxane (2 mL) and water (2 mL) were added K₂CO₃ (51.4 mg, 0.37 mmol) and Pd(dppf)Cl₂ (18.0 mg, 0.024 mmol). The reaction mixture was stirred at 90° C. for 2 h. After cooling down to rt, the mixture was diluted with H₂O (10 mL) and extracted with ethyl acetate (20 mL×3). The organic phase was washed with brine, dried over anhydrous Na₂SO₄, filtered, and concentrated. The residue was purified by silica gel column chromatography to provide the desired product (80.3 mg, 64% yield) as a yellow oil. MS (ESI) m/z -350.5 [M+H]⁺.

### Step 2. Synthesis of 2-((5-((tert-butoxycarbonyl)amino)pentyl)amino)-6-ethylnicotinic acid

To a mixture of 2-[5-(tert-butoxycarbonylamino)pentylamino]-6-vinyl-pyridine-3-carboxylic acid (40 mg, 0.11 mmol) in MeOH (3 mL) was added 10% Pd/C (13.9 mg, 0.11 mmol). The reaction mixture was stirred at rt under hydrogen atmosphere for 12 h. The mixture was filtered through Celite and the filtrate was concentrated under reduced pressure to provide the desired product (35 mg, 87% yield) as a yellow oil. This compound was used directly in the next step without further purification.

The remaining steps were performed according to the procedures for preparing CPD-31 to provide the desired product (2 mg, 6% yield over 3 steps) as a yellow solid. MS (ESI) m/z=859.9 [M+H]⁺.

### Example 109. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-(dimethylamino)cyclopentyl)-6-(methylamino)nicotinamide (CPD-55)

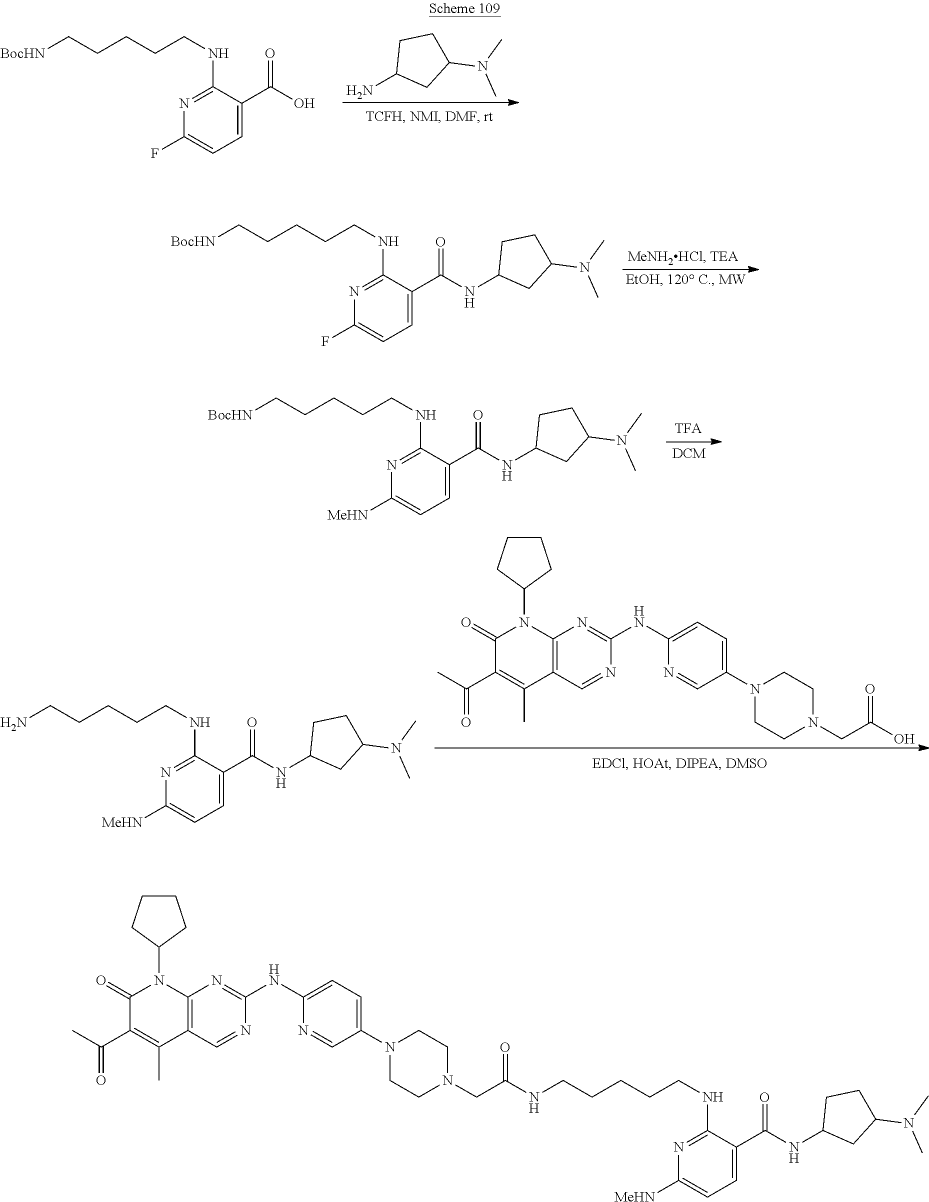

CPD-55 was synthesized following the procedures for preparing CPD-31 (16.2 mg, 16% yield over 4 steps) as a yellow solid. MS (ESI) m/z=850.9 [M+H]⁺.

### Example 110. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(4-cyclopropyl-5-methylthiazol-2-yl)-6-(methylamino)nicotinamide (CPD-56)

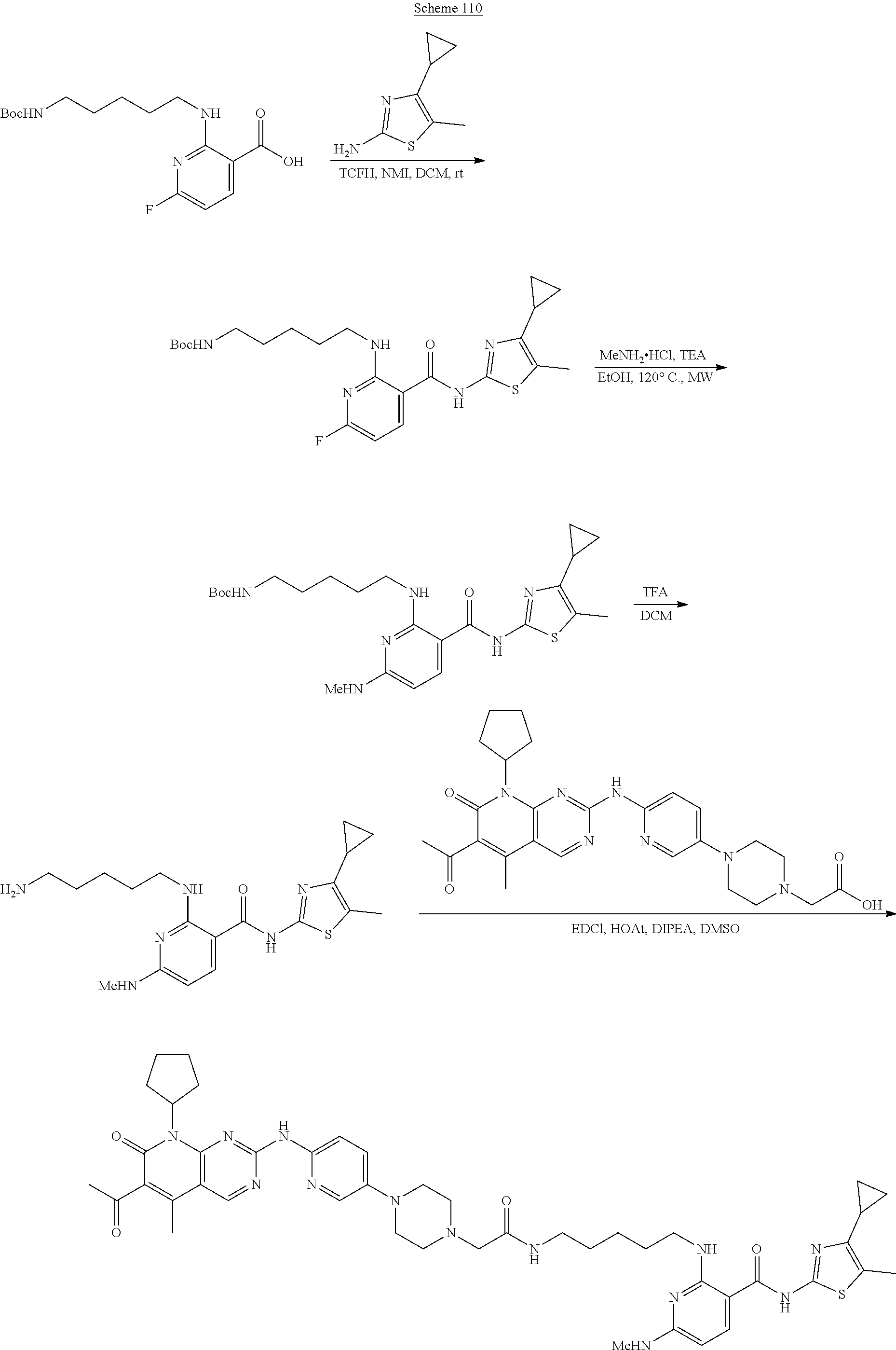

CPD-56 was synthesized following the procedures for preparing CPD-31 (5.5 mg, 12% yield over 4 steps) as a yellow solid. MS (ESI) m/z=876.7 [M+H]⁺.

### Example 111. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-methylpyridin-2-yl)-6-(methylamino)nicotinamide (CPD-57)

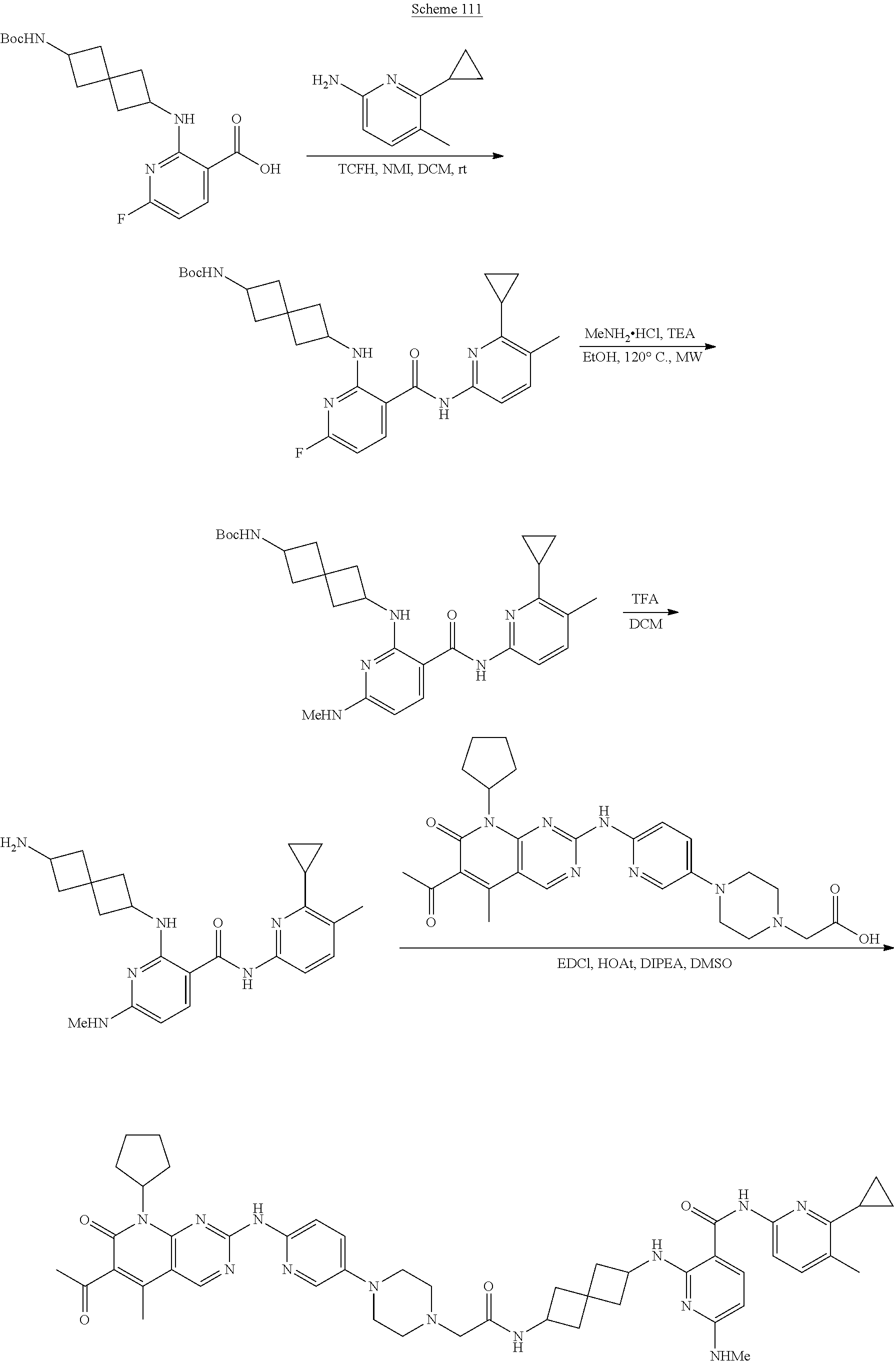

CPD-57 was synthesized following the procedures for preparing CPD-31 (10.2 mg, 16% yield over 4 steps) as a yellow solid. MS (ESI) m/z=894.9 [M+H]⁺.

### Example 112. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridin-3-yl)-6-(methylamino)nicotinamide (CPD-58)

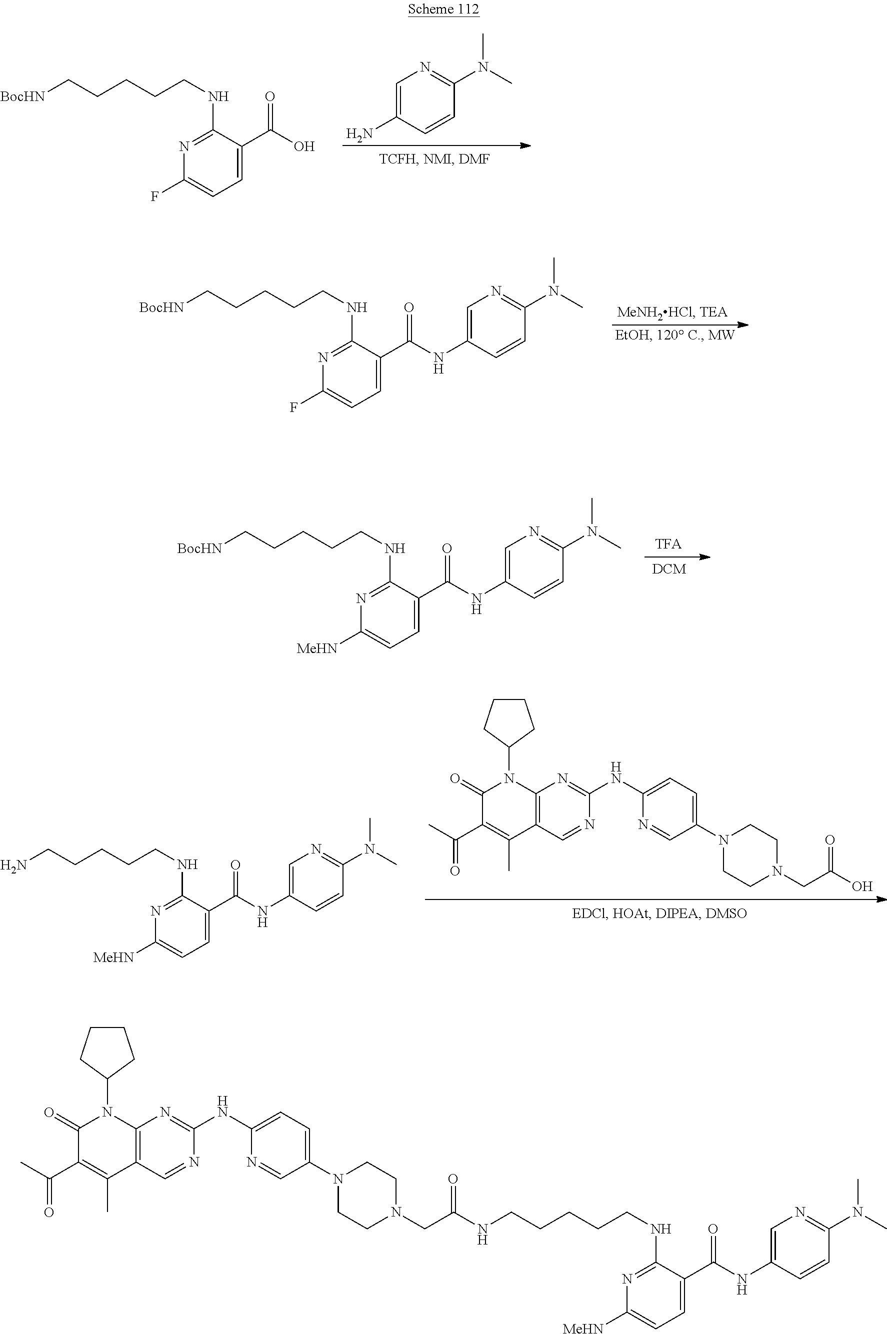

CPD-58 was synthesized following the procedures for preparing CPD-31 (8 mg, 11% yield over 4 steps) as a yellow solid. MS (ESI) m/z=859.7 [M+H]⁺.

### Example 113. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(cyclobutylamino)-N-(6-(dimethylamino)pyridazin-3-yl)nicotinamide (CPD-59)

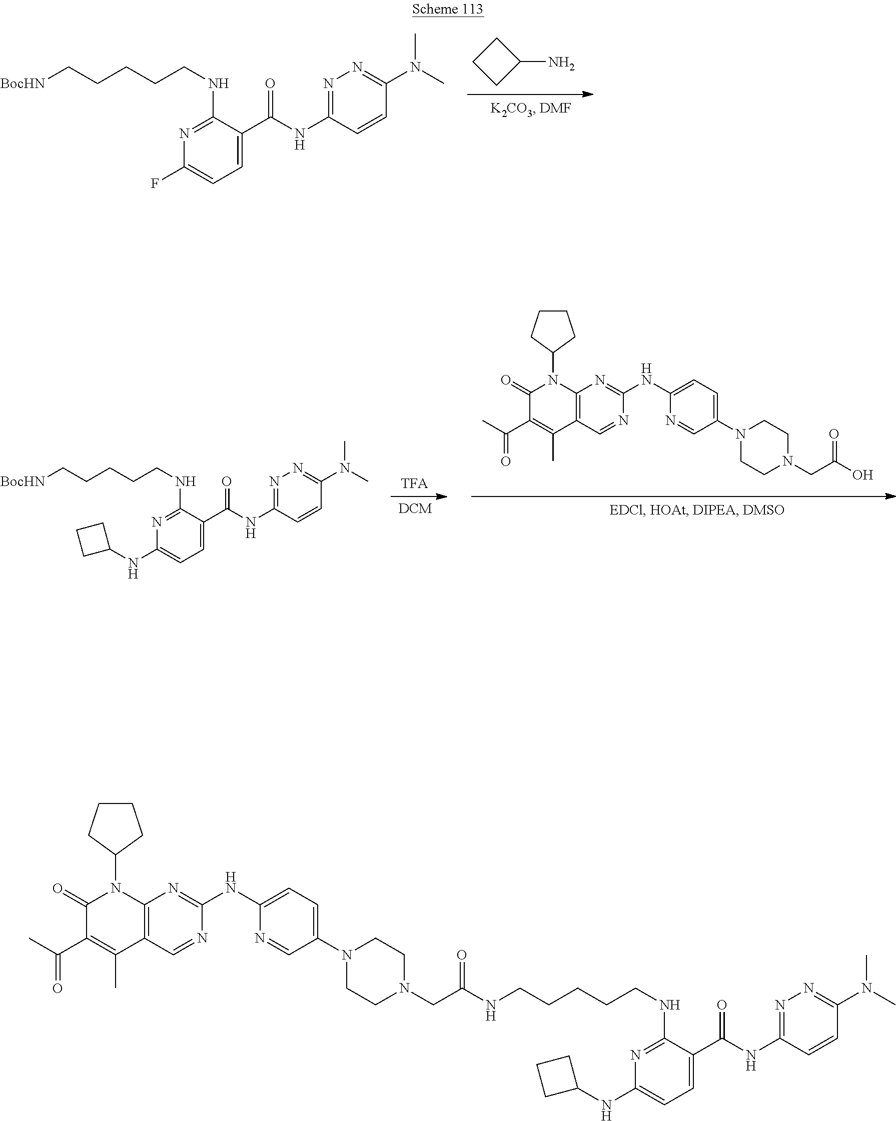

### Step 1. Synthesis of tert-butyl (5-((6-(cyclobutylamino)-3-((6-(dimethylamino)pyridazin-3-yl)carbamoyl)pyridin-2-yl)amino)pentyl)carbamate

To a solution of tert-butyl (5-((3-((6-(dimethylamino)pyridazin-3-yl)carbamoyl)-6-fluoropyridin-2-yl)amino)pentyl)carbamate (30 mg, 0.065 mmol) in DMF (3 mL) were added cyclobutanamine (23 mg, 0.33 mmol) and K₂CO₃ (45 mg, 0.33 mmol) at rt. The reaction mixture was stirred 90° C. for 6 h. After cooling down to rt, the reaction mixture was poured into water (20 mL) and extracted with ethyl acetate (3×20 mL). The combined organic layers were washed with saturated brine (20 mL), dried over anhydrous sodium sulfate, filtered, and evaporated under reduced pressure. The resulting residue was purified by silica gel chromatography to provide the desired product (18 mg, 54% yield) as a light-yellow solid. MS (ESI) m/z=513.7 [M+H]⁺.

The remaining steps were performed according to the standard procedures to provide the desired product (5 mg, 16% yield over 2 steps) as a yellow solid. MS (ESI) m/z=900.8 [M+H]⁺.

### Example 114. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-6-cyclopropyl-N-(6-cyclopropyl-5-methylpyridin-2-yl)nicotinamide (CPD-60)

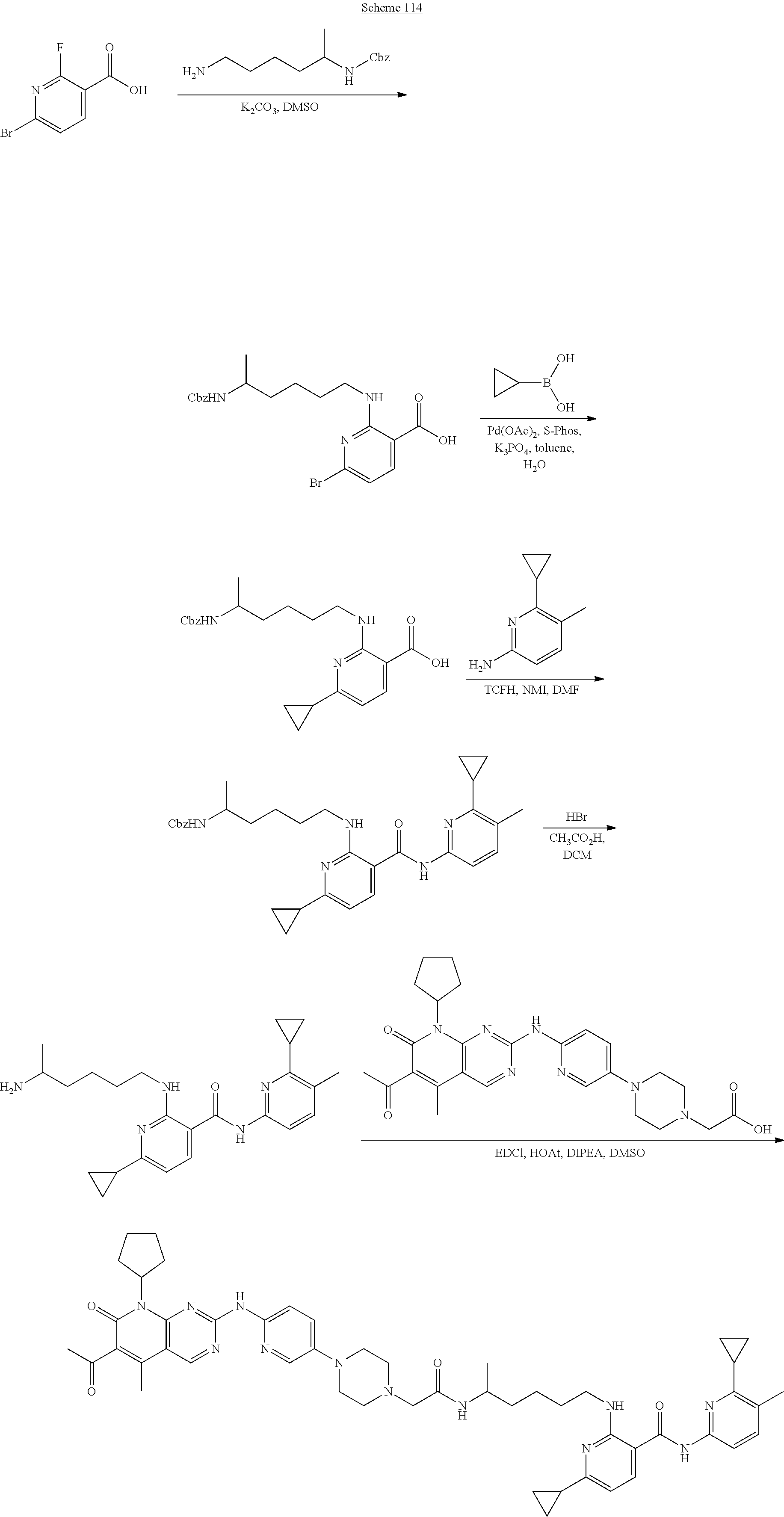

### Step 1. Synthesis of 2-((5-(((benzyloxy)carbonyl)amino)hexyl)amino)-6-bromonicotinic acid

To a solution of 6-bromo-2-fluoronicotinic acid (50 mg, 0.2 mmol) and benzyl (6-aminohexan-2-yl)carbamate (56 mg, 0.2 mmol) in DMSO (5 mL) was added K₂CO₃ (94 mg, 0.6 mmol) at rt. The reaction mixture was stirred at 100° C. for 16 h. After cooling down to rt, the reaction mixture was poured into water (30 mL) and extracted with DCM (20 mL×3). The combined organic layers were washed with saturated brine, dried over anhydrous sodium sulfate, filtered, and evaporated under reduced pressure. The residue was purified by reverse-phase chromatography to provide the desired product (56 mg, 54% yield) as a colorless oil. MS (ESI) m/z=450.4 [M+H]⁺.

### Step 2. Synthesis of 2-((5-(((benzyloxy)carbonyl)amino)hexyl)amino)-6-cyclopropylnicotinic acid

To a mixture of 2-((5-(((benzyloxy)carbonyl)amino)hexyl)amino)-6-bromonicotinic acid (100 mg, 0.2 mmol) and cyclopropylboronic acid (19 mg, 0.2 mmol) in toluene (4 mL) and H₂O (1 mL) were added K₃PO₄ (141 mg, 0.6 mmol), S-Phos(9 mg, 0.02 mmol) and Pd(OAc)₂ (5 mg, 0.02 mmol) at rt under N₂.

The reaction mixture was stirred at 100° C. for 16 h. After cooling down to rt, the reaction mixture was concentrated and purified by reverse-phase chromatography to provide the desired product (45 mg, 49% yield) as a yellow solid. MS (ESI) m/z=412.8 [M+H]⁺.

### Step 3. Synthesis of benzyl (6-((6-cyclopropyl-3-((6-cyclopropyl-5-methylpyridin-2-yl)carbamoyl)pyridin-2-yl)amino)hexan-2-yl)carbamate

To a mixture of 6-cyclopropyl-5-methyl-pyridin-2-amine (9 mg, 0.06 mmol) and 2-((5-(((benzyloxy)carbonyl)amino)hexyl)amino)-6-bromonicotinic acid (24 mg, 0.06 mmol) in DMF (3 mL) were added 1-methylimidazole (5 mg, 0.06 mmol), TCFH (18 mg, 0.06 mmol) at rt. After the reaction mixture was stirred at rt for 2 h, it was concentrated and purified by reverse-phase chromatography to provide the desired product (15 mg, 46% yield) as a yellow solid. MS (ESI) m/z=542.7 [M+H]⁺.

### Step 4. Synthesis of 2-((5-aminohexyl)amino)-6-cyclopropyl-N-(6-cyclopropyl-5-methylpyridin-2-yl)nicotinamide

To a solution of benzyl (6-((6-cyclopropyl-3-((6-cyclopropyl-5-methylpyridin-2-yl)carbamoyl)pyridin-2-yl)amino)hexan-2-yl)carbamate (15 mg, 0.028 mmol) in DCM (3 mL) were added hydrogen bromide (6.7 mg, 0.083 mmol) and CH₃CO₂H (16 mg, 0.028 mmol) at 0° C. After stirring at rt for 1 h, the mixture was concentrated and purified by reverse-phase chromatography to provide the desired product (6 mg, 53% yield) as a yellow solid. MS (ESI) m/z=408.6 [M+H]⁺.

### Step 5. Synthesis of 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)hexyl)amino)-6-cyclopropyl-N-(6-cyclopropyl-5-methylpyridin-2-yl)nicotinamide

The title compound was synthesized following the standard procedure for preparing CPD-1 (2.0 mg, 30% yield) as a light-yellow solid. MS (ESI) m/z=895.8 [M+H]⁺.

### Example 115. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-3-methylpyridin-2-yl)-6-(methylamino)nicotinamide (CPD-61)

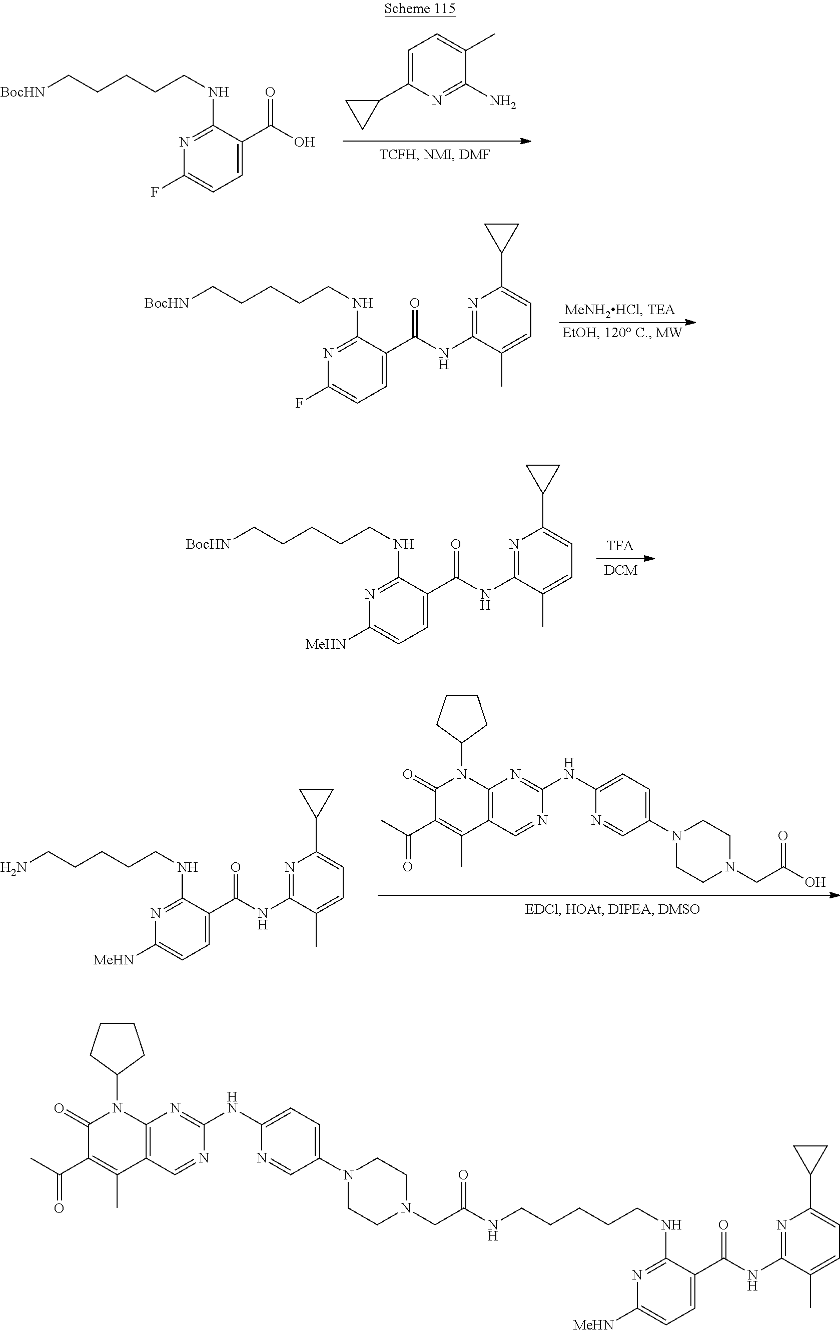

CPD-61 was synthesized following the procedures for preparing CPD-31 (3.0 mg, 1% yield over 4 steps) as a yellow solid. MS (ESI) m/z=870.9 [M+H]⁺.

### Example 116. 2-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-(methylamino)nicotinamide (CPD-62)

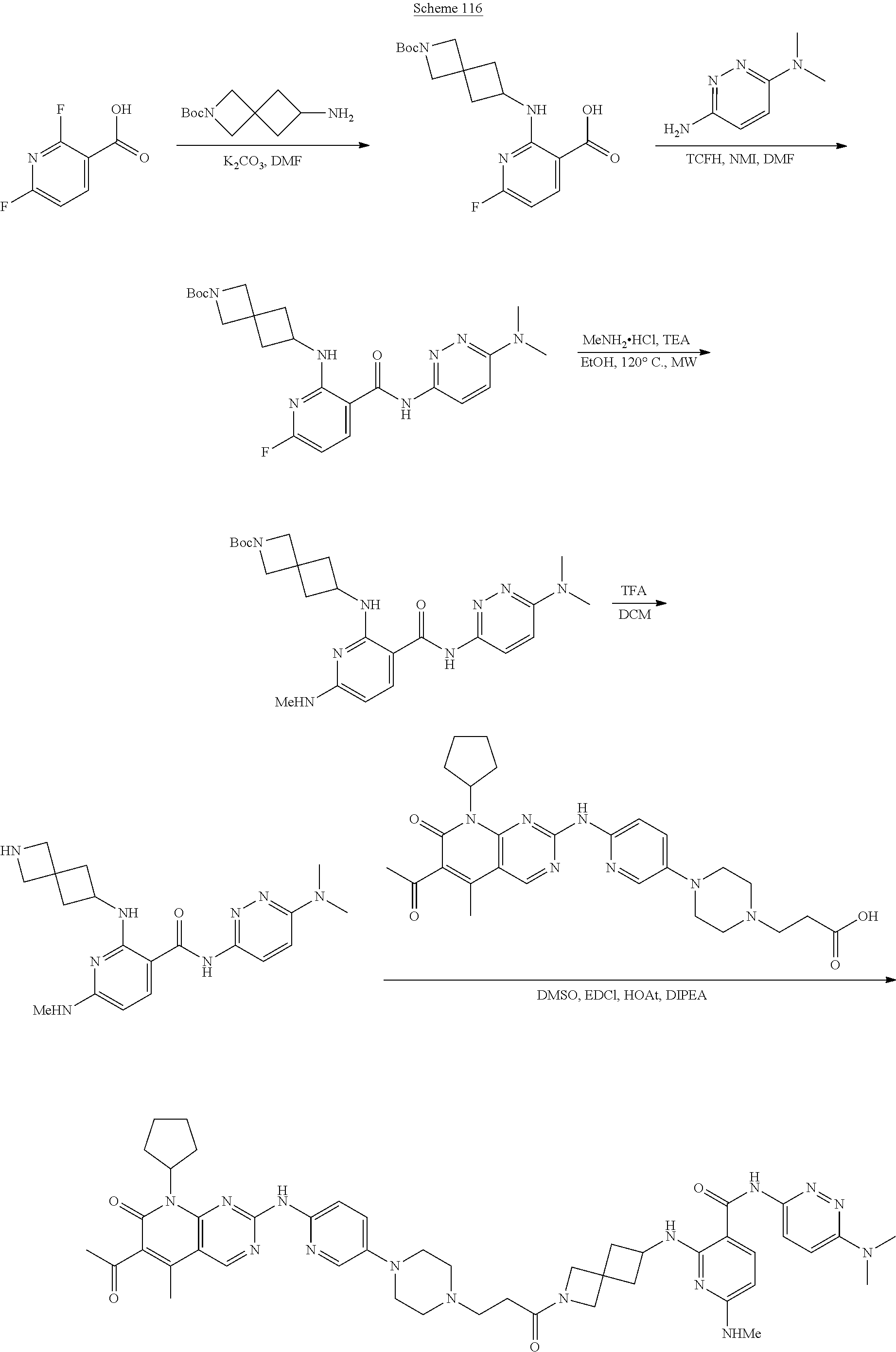

CPD-62 was synthesized following the procedures for preparing CPD-39 and CPD-31 (3 mg, 6% yield over 4 steps) as a yellow solid. MS (ESI) m/z=884.8 [M+H]⁺.

### Example 117. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-isopropylnicotinamide (CPD-63)

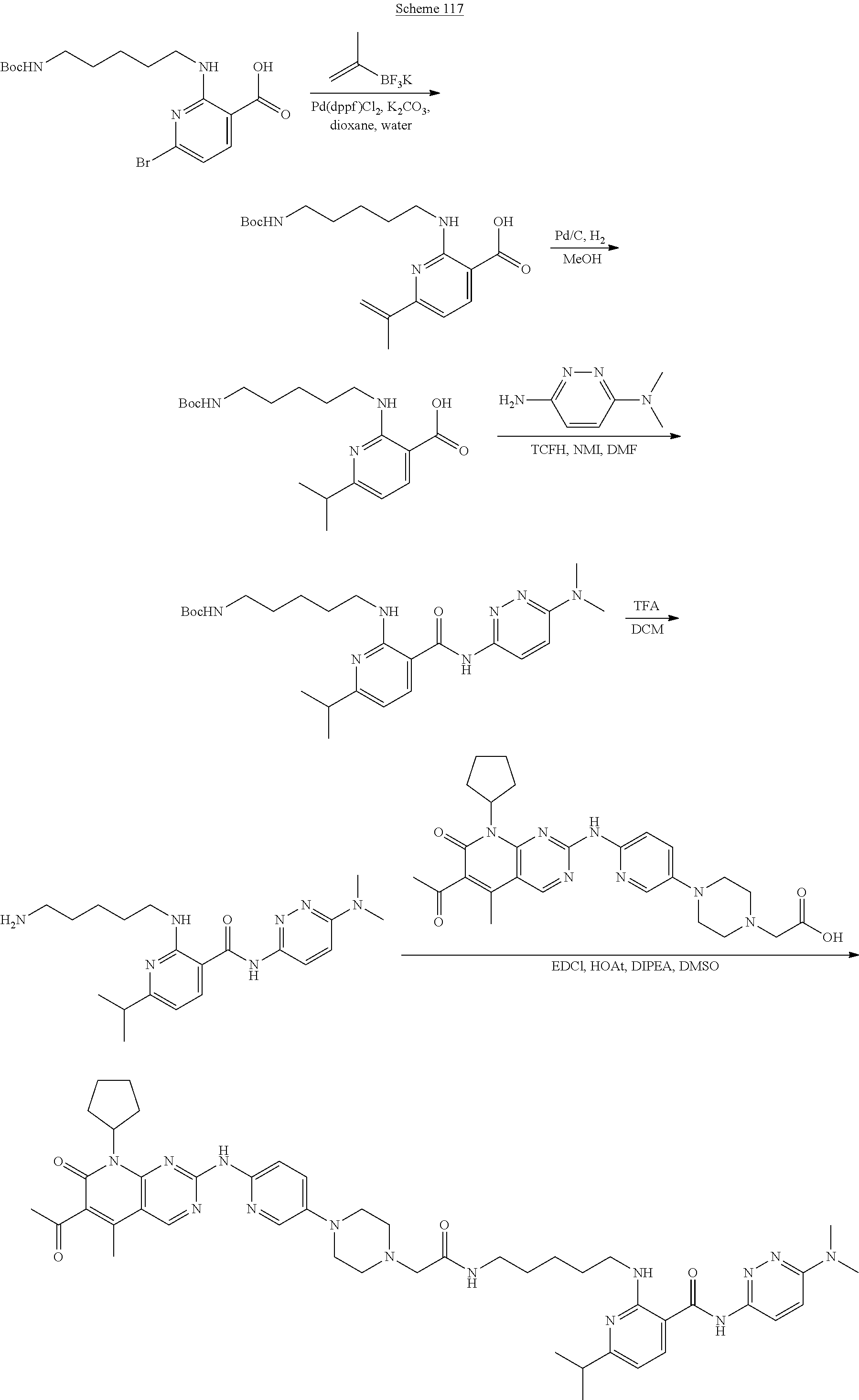

CPD-63 was synthesized following the procedures for preparing CPD-54 (2.5 mg, 2% yield over 5 steps) as a yellow solid. MS (ESI) m/z=873.9 [M+H]⁺.

### Example 118. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-(dimethylamino)cyclobutyl)-6-(methylamino)nicotinamide (CPD-64)

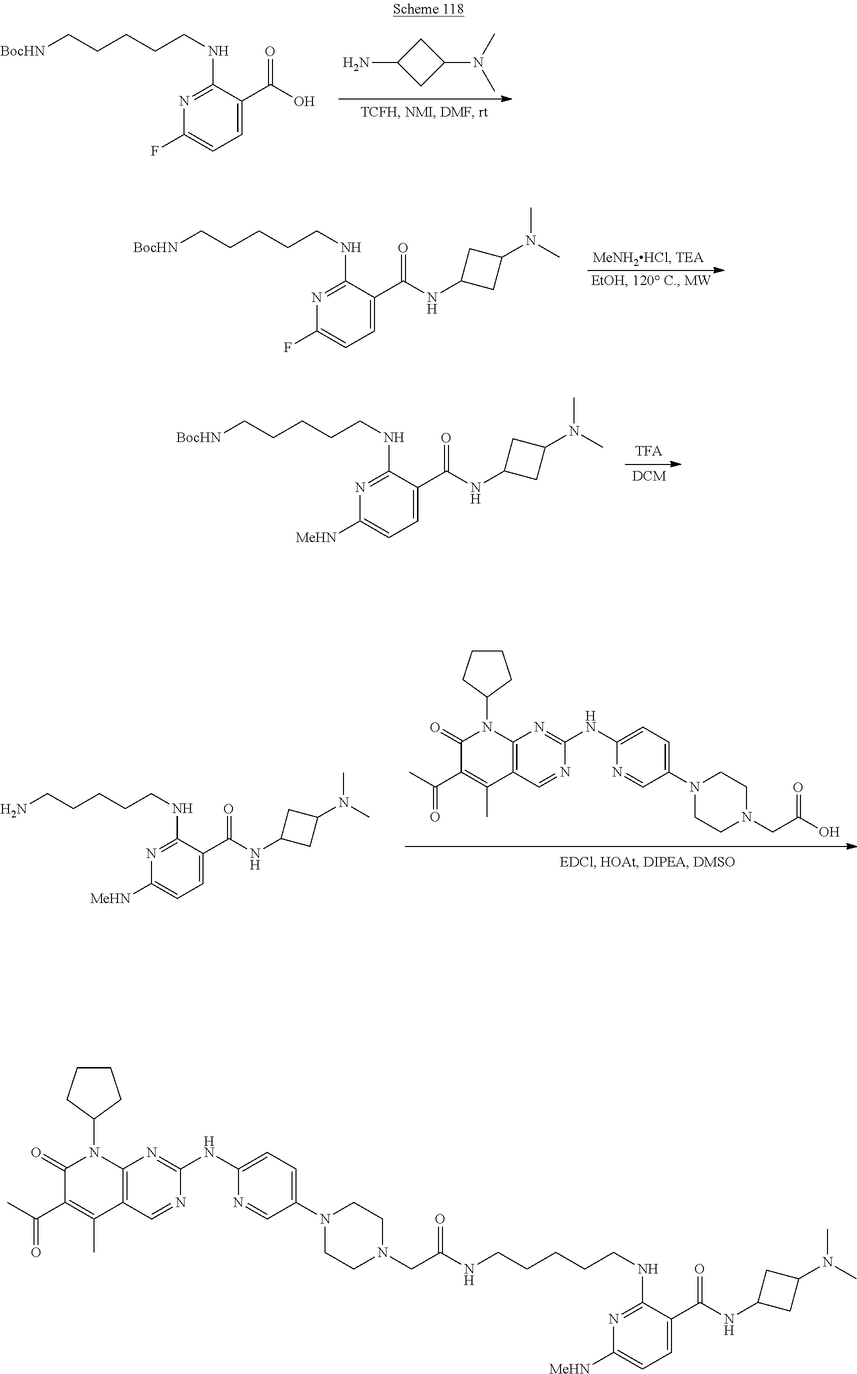

CPD-64 was synthesized following the procedures for preparing CPD-31 (16.6 mg, 7% yield over 4 steps) as a yellow solid. MS (ESI) m/z=836.8 [M+H]⁺.

### Example 119. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(3-((dimethylamino)methyl)cyclobutyl)-6-(methylamino)nicotinamide (CPD-65)

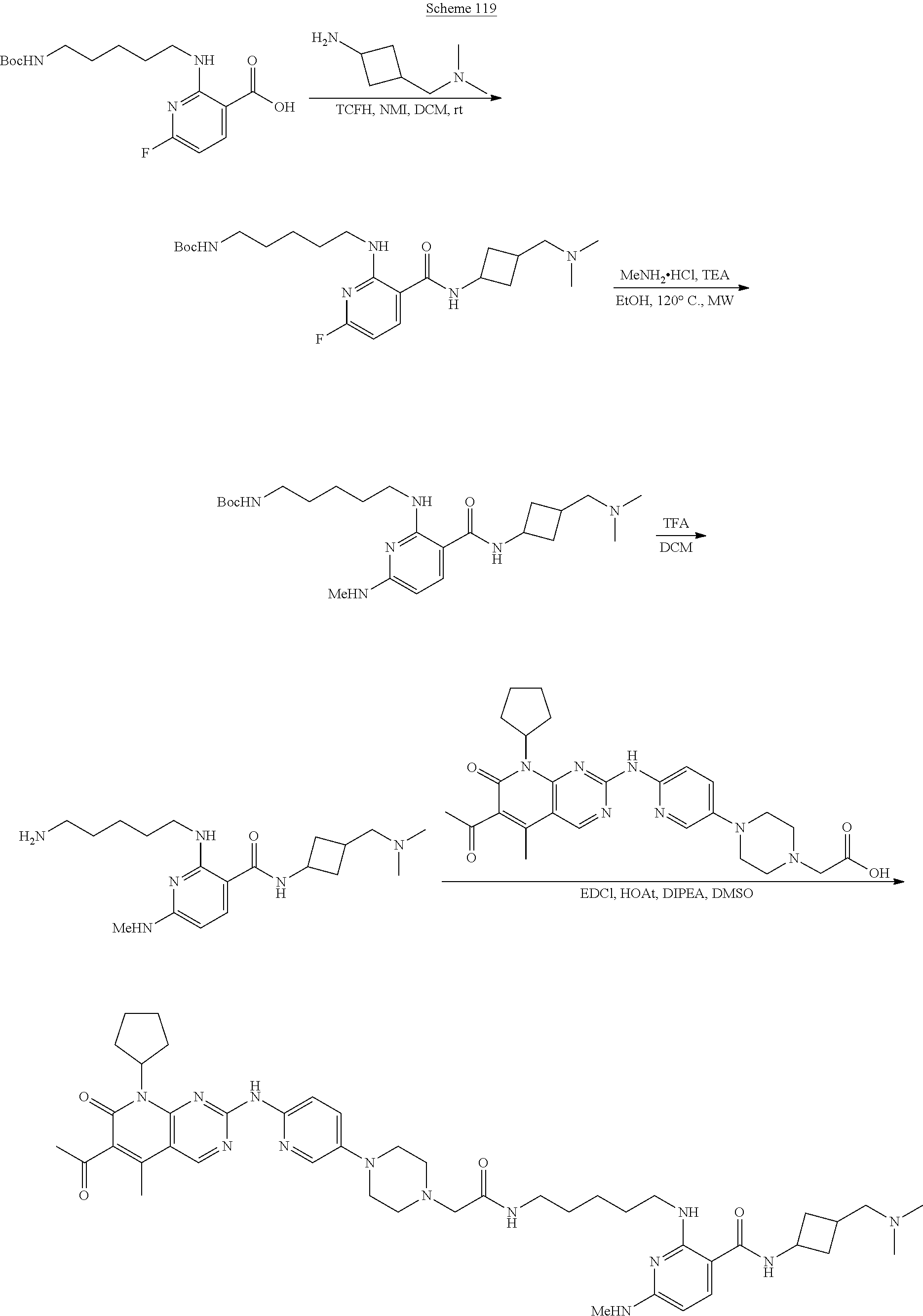

CPD-65 was synthesized following the procedures for preparing CPD-31 (15.3 mg, 6% yield over 4 steps) as a yellow solid. MS (ESI) m/z=851.0 [M+H]⁺.

### Example 120. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-((methyl-d₃)amino)nicotinamide (CPD-66)

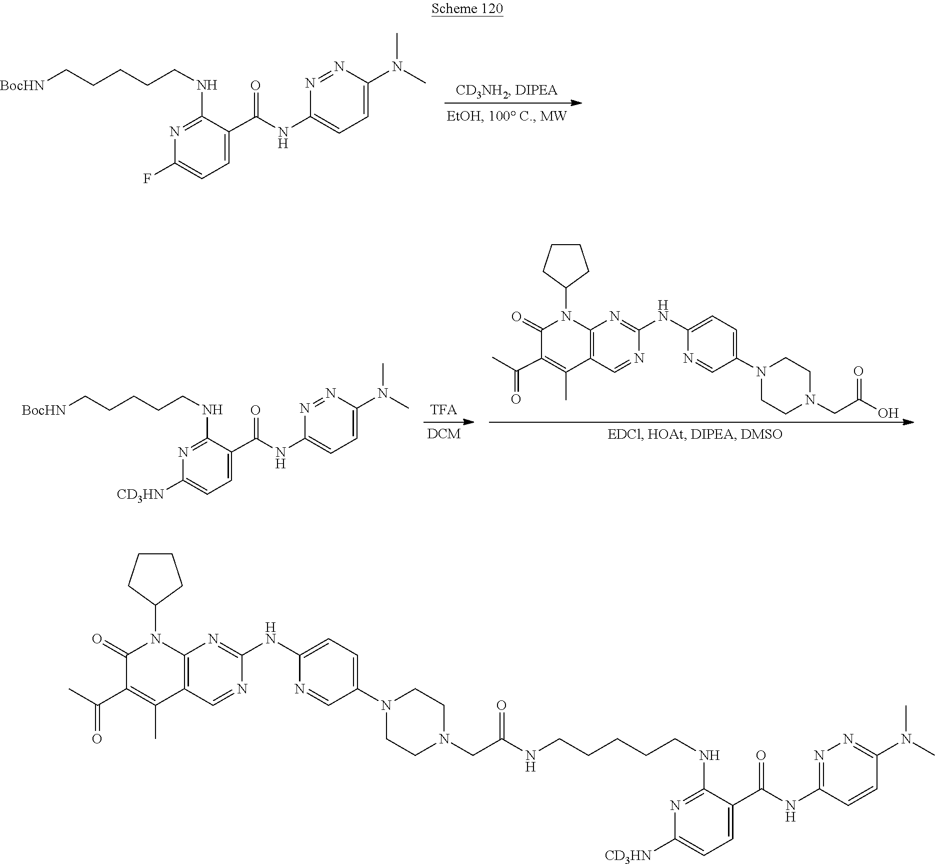

CPD-66 was synthesized following the procedures for preparing CPD-31 (2.8 mg, 5% yield over 3 steps) as a yellow solid. MS (ESI) m/z=863.8 [M+H]⁺.

### Example 121. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)spiro[3.3]heptan-2-yl)-6-(methylamino)nicotinamide (CPD-67)

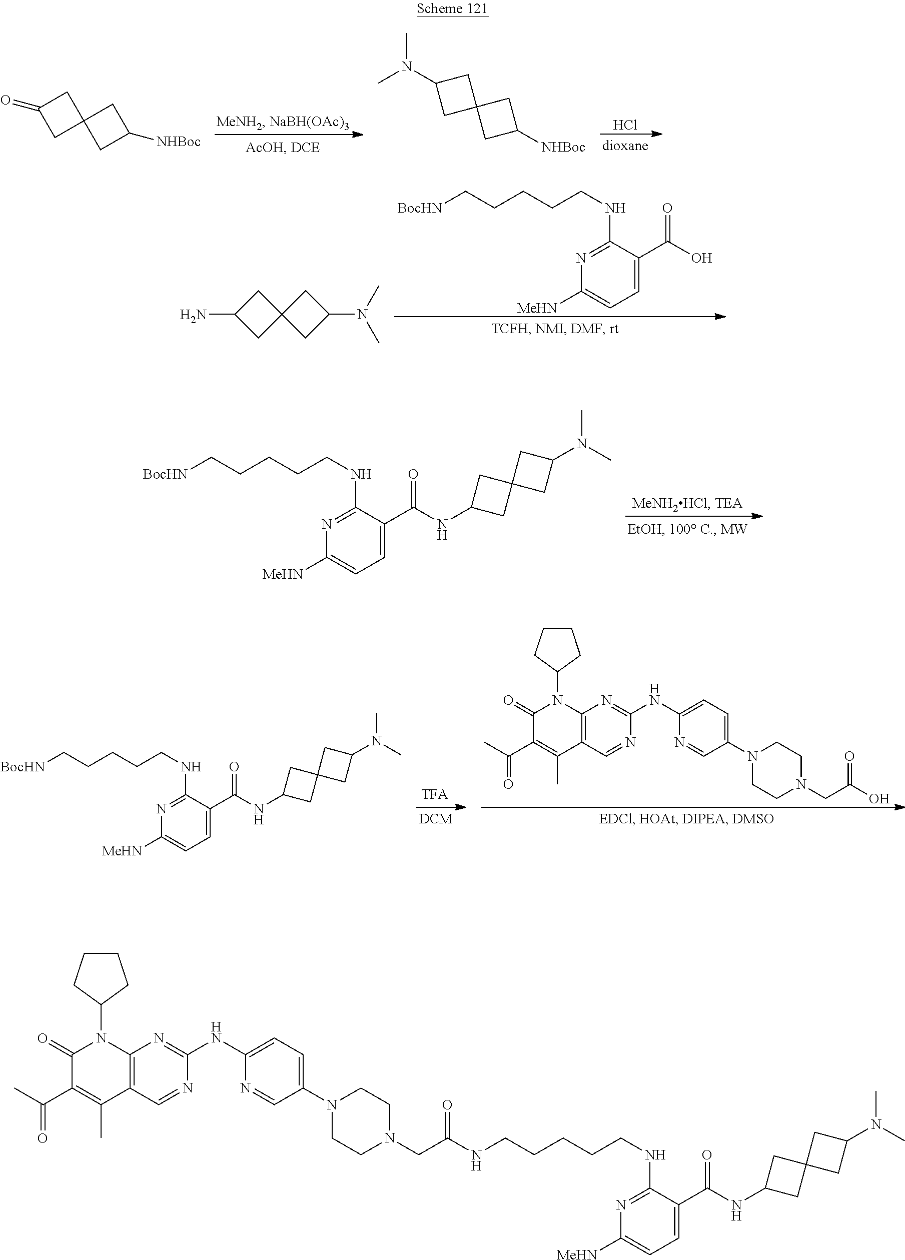

### Step 1. Synthesis of tert-butyl (6-(dimethylamino)spiro[3.3]heptan-2-yl)carbamate

To a solution of tert-butyl N-(2-oxospiro[3.3]heptan-6-yl)carbamate (80 mg, 0.355 mmol), N-methylmethanamine (160.1 mg, 3.55 mmol) and acetic acid (21.3 mg, 0.355 mmol) in DCE (5 mL) was added sodium triacetoxyborohydride (150.5 mg, 0.71 mmol) in portions at rt. After the reaction mixture was stirred at rt for 1 h, it was quenched with H₂O (20 mL) and extracted with dichloromethane (20 mL×3). The combined organic phase was washed with brine, dried over anhydrous Na₂SO₄, filtered, and concentrated to provide the desired product (80 mg, crude, 89% yield) as a yellow oil. MS (ESI) m/z
255.6 [M+H]⁺.

### Step 2. Synthesis of N²,N²-dimethylspiro[3.3]heptane-2,6-diamine

To a solution of tert-butyl N-[2-(dimethylamino)spiro[3.3]heptan-6-yl]carbamate (80 mg, 0.31 mmol) in dioxane (2 mL) was added HCl solution (4 M in dioxane, 0.1 mL, 0.4 mmol). After the reaction mixture was stirred at rt for 1 h, it was concentrated under vacuum to provide the desired product (45 mg, crude, 93% yield) as a white solid.

The remaining steps were performed according to the procedures for preparing CPD-31 to provide the desired product (12.3 mg, 19% yield over 4 steps) as a yellow solid. MS (ESI) m/z=876.9 [M+H]⁺.

### Example 122. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-6-(trifluoromethyl)nicotinamide (CPD-68)

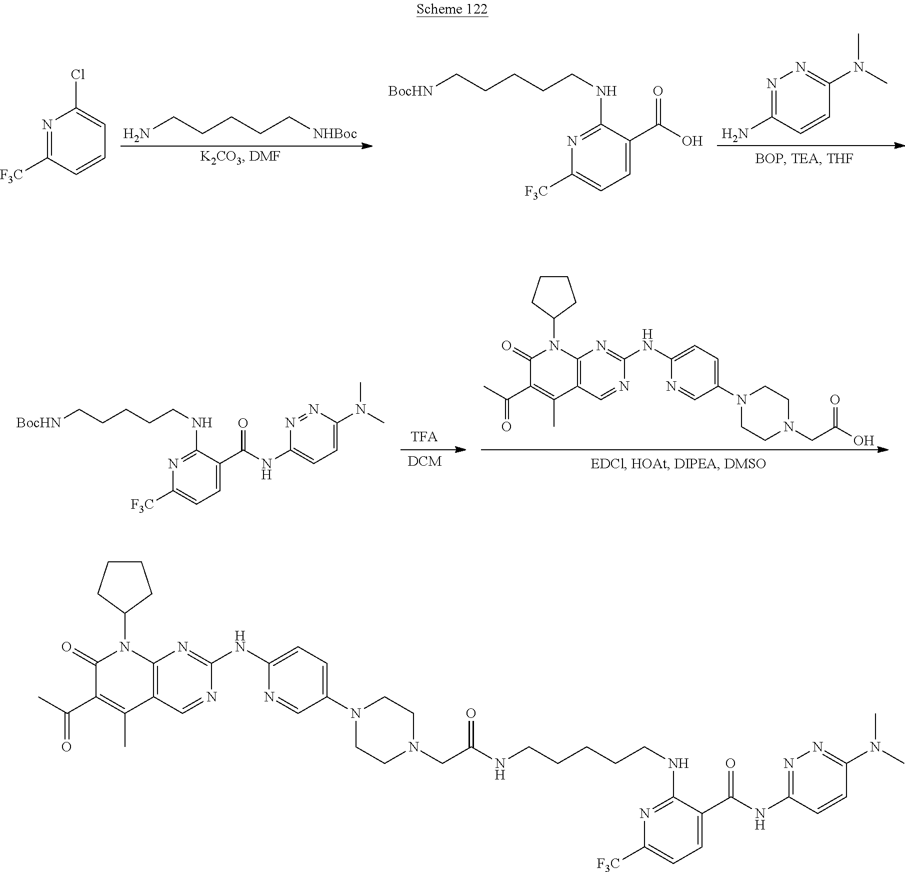

CPD-68 was synthesized following the procedures for preparing CPD-39 and CPD-34 (3.3 mg, 4% yield over 4 steps) as a yellow solid. MS (ESI) m/z=899.7 [M+H]⁺.

### Example 123. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6,7-dihydro-5H-cyclopenta[b]pyridin-2-yl)-6-(methylamino)nicotinamide (CPD-69)

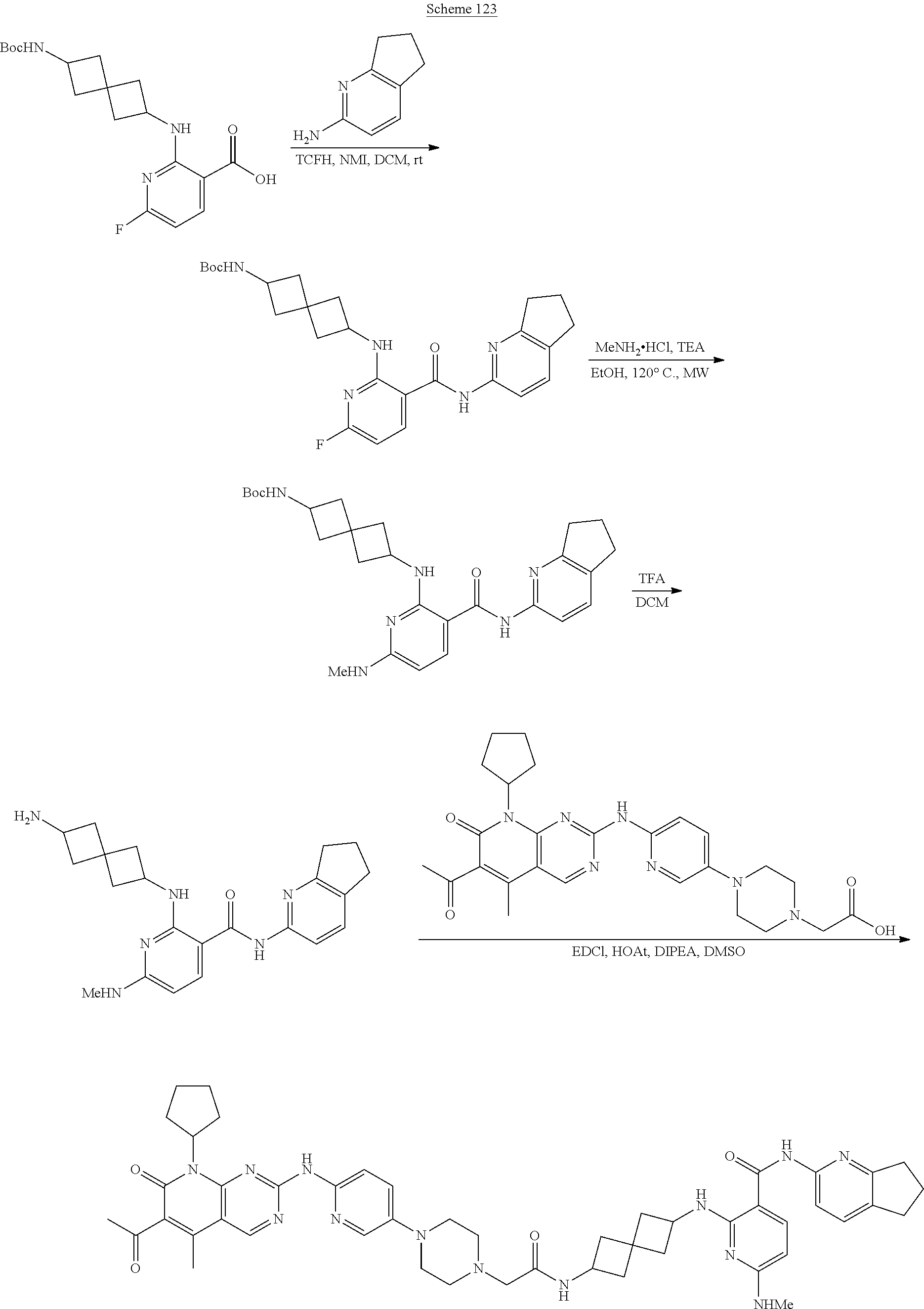

CPD-69 was synthesized following the procedures for preparing CPD-31 (2.4 mg, 4% yield over 4 steps) as a yellow solid. MS (ESI) m/z=880.9 [M+H]⁺.

### Example 124. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-(methylamino)nicotinamide (CPD-70)

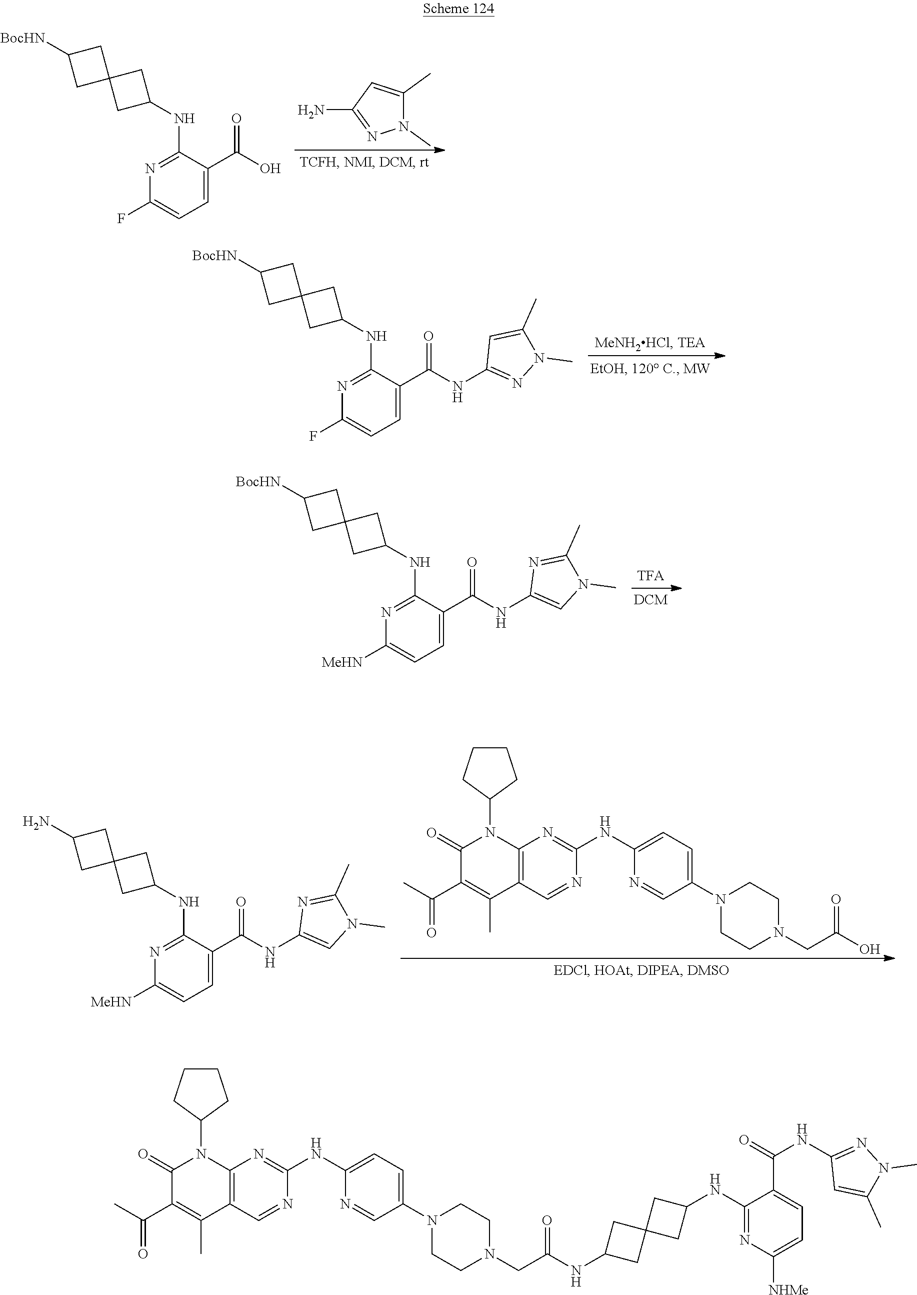

CPD-70 was synthesized following the procedures for preparing CPD-31 (5.2 mg, 11% yield over 4 steps) as a yellow solid. MS (ESI) m/z=857.8 [M+H]⁺.

### Example 125. 2-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-N-methyl-6-(methylamino)nicotinamide (CPD-71)

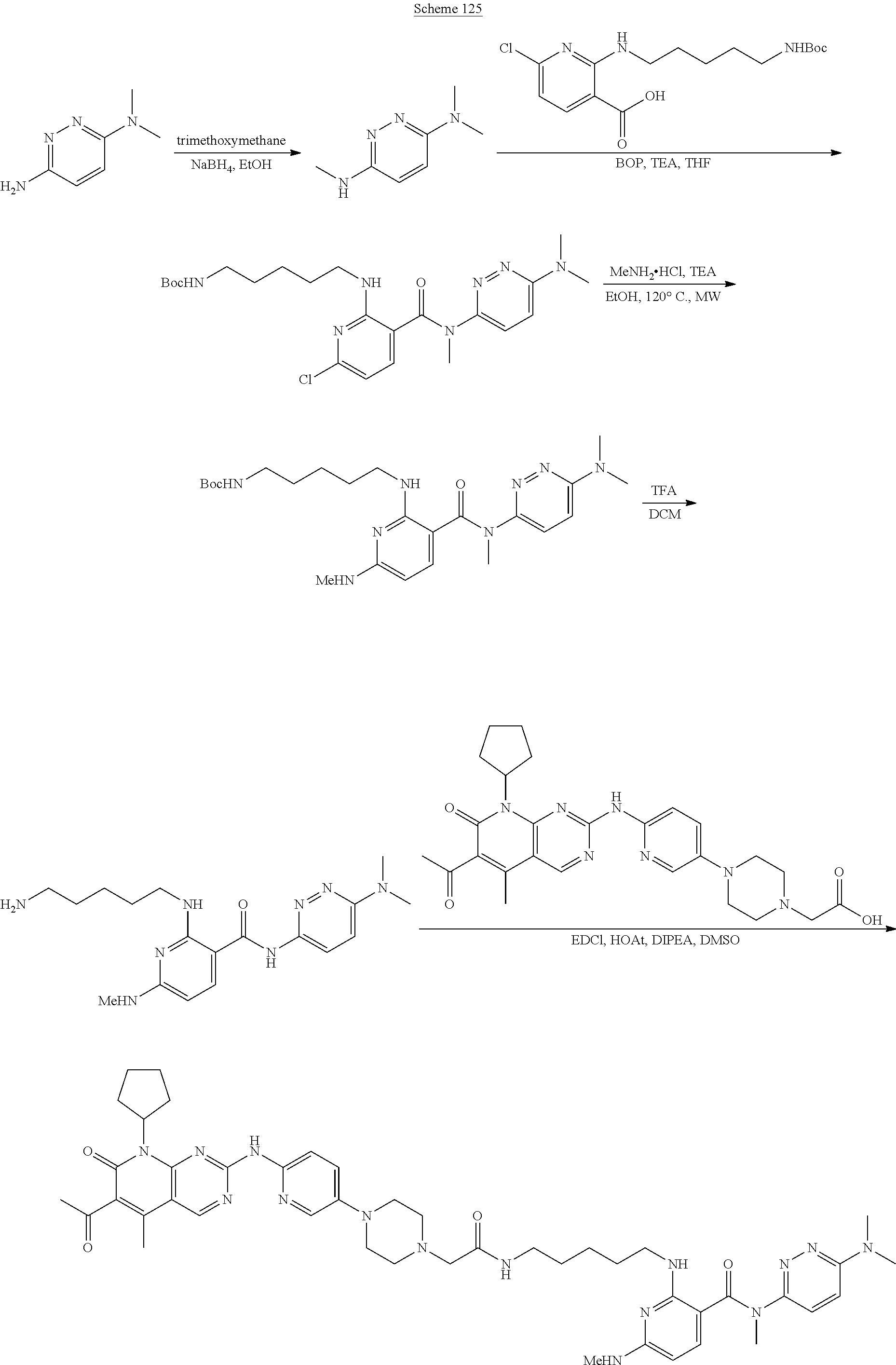

### Step 1. Synthesis of N³,N³, N⁶-trimethylpyridazine-3, 6-diamine

To a solution of N³,N³-dimethylpyridazine-3, 6-diamine (100.0 mg, 0.72 mmol), trimethoxymethane (768.0 mg, 7.2 mmol) in ethanol (5 ml) was added NaBH₄ (22.4 mg, 0.72 mmol) at rt. The reaction mixture was stirred at 80 [symbol] for 2 h. After cooling down to rt, the reaction mixture was quenched with H₂O (20 mL) and extracted with ethyl acetate (20 mL×3). The organic phase was washed with brine, dried with anhydrous Na₂SO₄, filtered, and concentrated. The residue was purified by silica gel column chromatography to provide the desired product (80.0 mg, 73% yield) as a light-yellow solid. MS (ESI) m/z=153.1 [M+H]⁺.

The remaining steps were performed according to the procedures for preparing CPD-34 to provide the desired product (1.6 mg, 6% yield over 4 steps) as a light-yellow solid. MS (ESI) m/z=874.8 [M+H]⁺.

### Example 126. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(methylamino)nicotinamide (CPD-72)

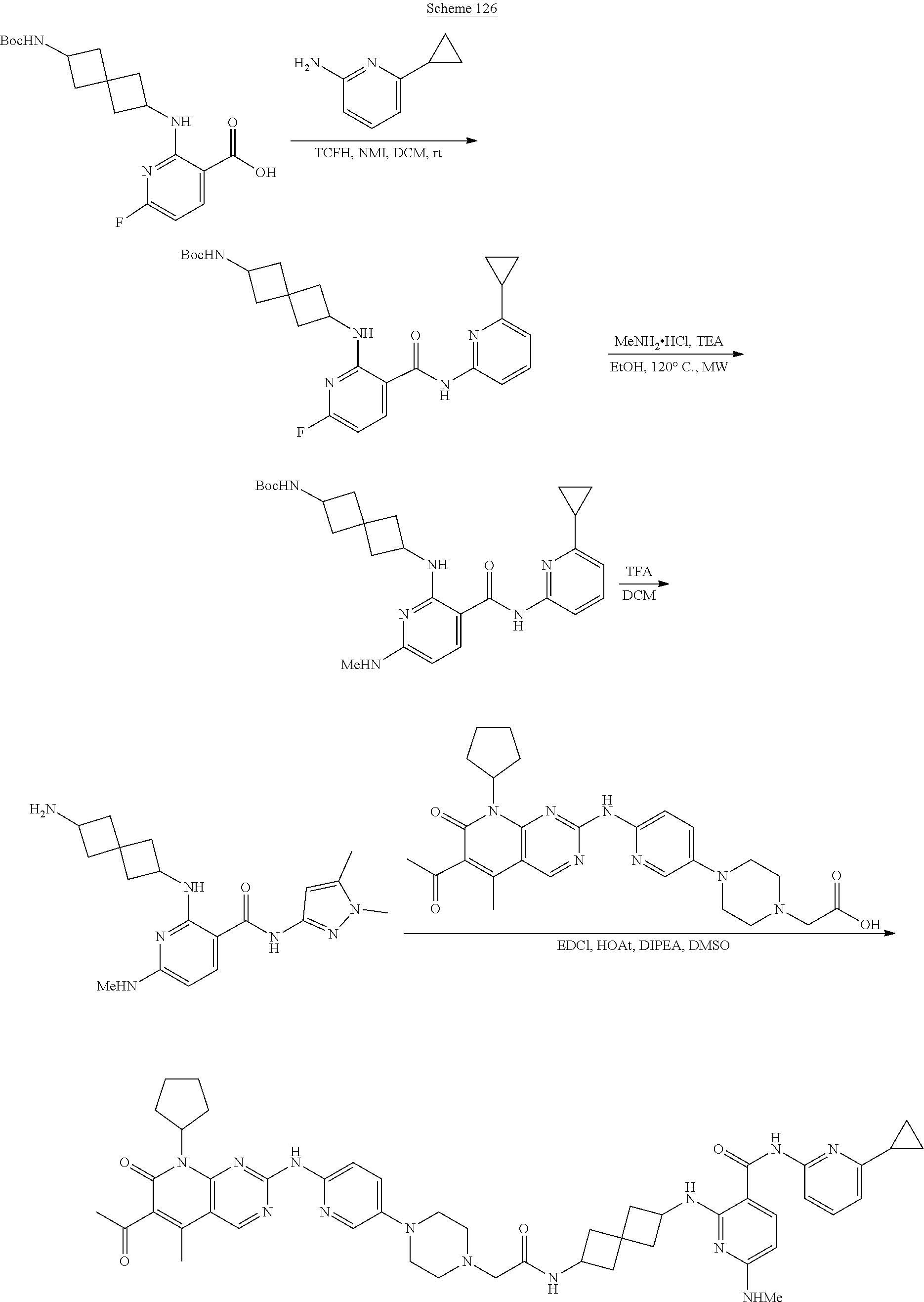

CPD-72 was synthesized following the procedures for preparing CPD-31 (3.1 mg, 8% yield over 4 steps) as a yellow solid. MS (ESI) m/z=880.9 [M+H]⁺.

### Example 127. 2-((trans-4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-(dimethylamino)pyridazin-3-yl)-6-(methylamino)nicotinamide (CPD-73)

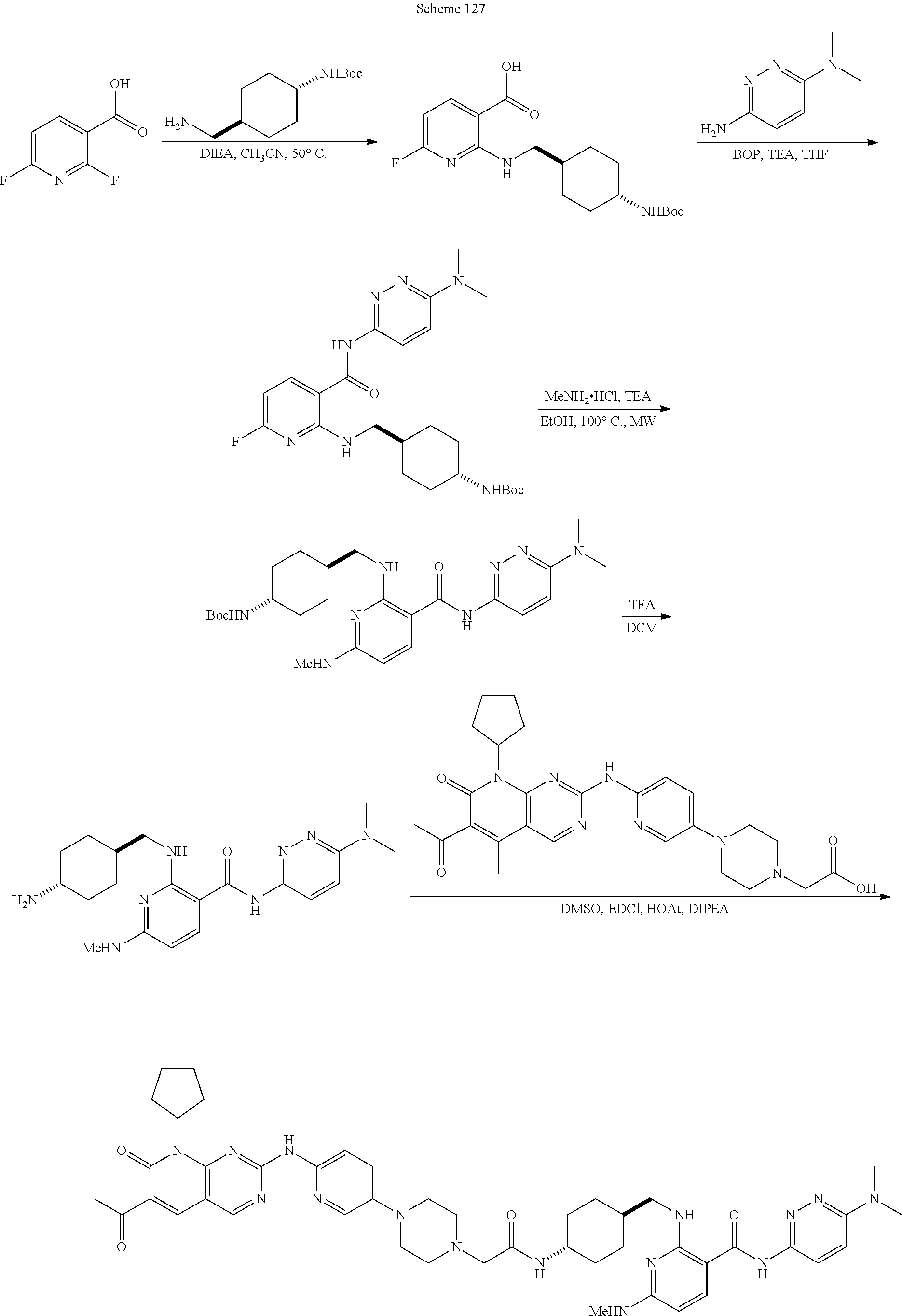

CPD-73 was synthesized following the procedures for preparing CPD-34 (5.6 mg, 5% yield over 5 steps) as a yellow solid. MS (ESI) m/z=886.9 [M+H]⁺.

### Example 128. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-3-yl)-6-(methylamino)nicotinamide (CPD-74)

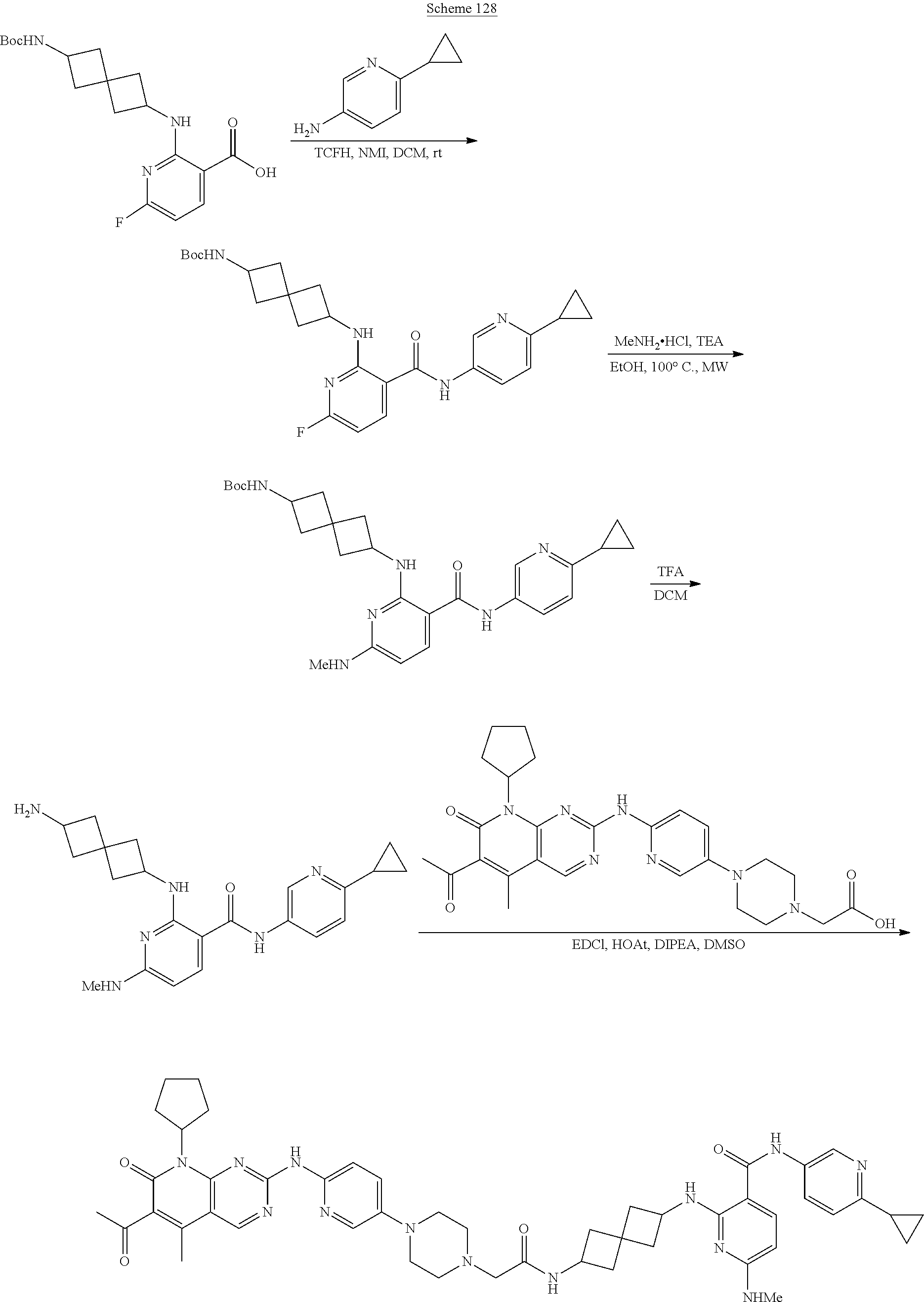

CPD-74 was synthesized following the procedures for preparing CPD-31 (1.2 mg, 1% yield over 4 steps) as a yellow solid. MS (ESI) n/z=880.9 [M+H]⁺.

### Example 129. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-methylpyridin-2-yl)nicotinamide (CPD-75)

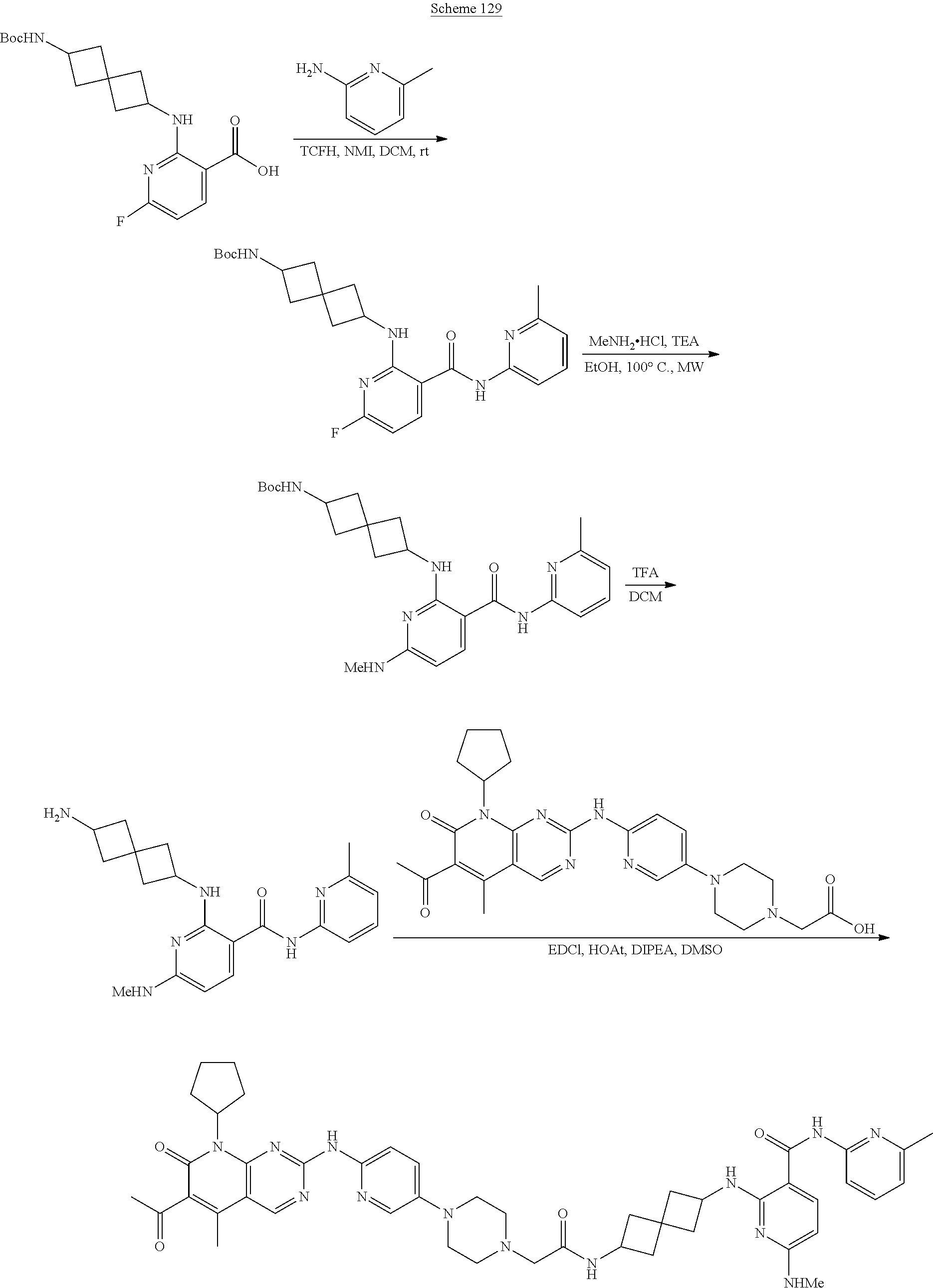

CPD-75 was synthesized following the procedures for preparing CPD-31 (5.3 mg, 7% yield over 4 steps) as a yellow solid. MS (ESI) m/z=854.9 [M+H]⁺.

### Example 130. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-isopropylpyridin-2-yl)-6-(methylamino)nicotinamide (CPD-76)

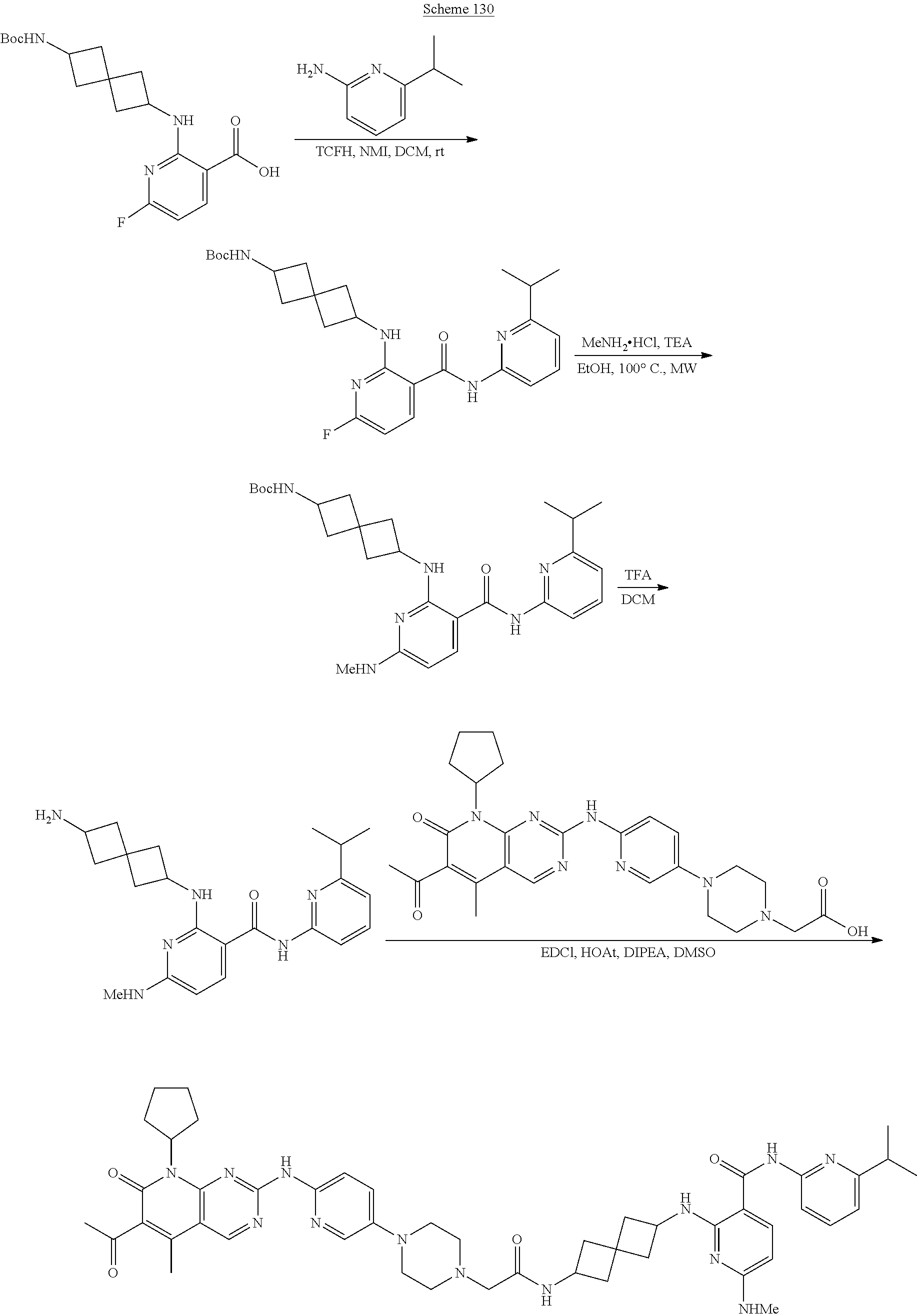

CPD-76 was synthesized following the procedures for preparing CPD-31 (3.6 mg, 5.10% yield over 4 steps) as a yellow solid. MS (ESI) n/z=882.9 [M+H]⁺.

### Example 131. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-oxo-1,6-dihydropyridazin-3-yl)nicotinamide (CPD-77)

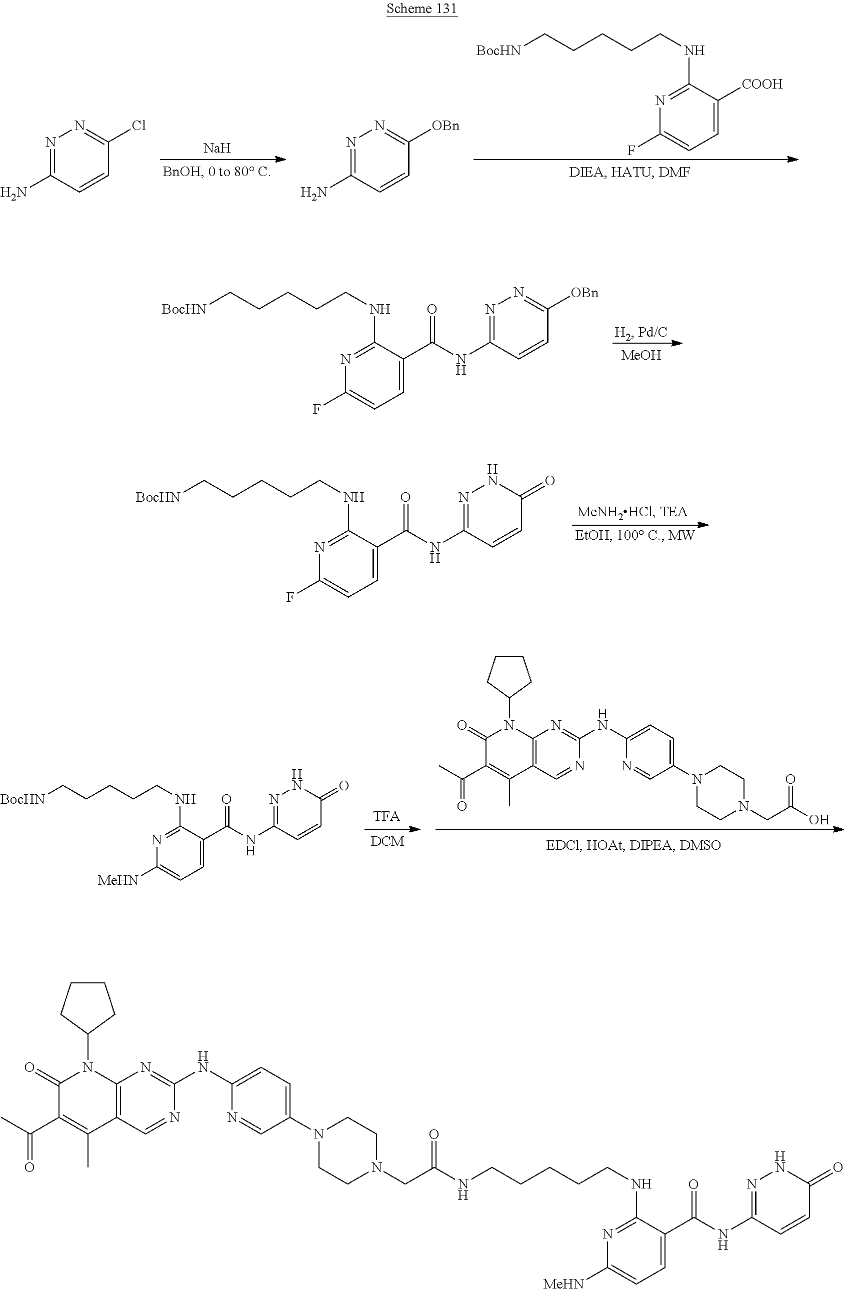

### Step 1. Synthesis of 6-(benzyloxy)pyridazin-3-amine

To a solution of 6-chloropyridazin-3-amine (3.0 g, 23.1 mmol) in BnOH (72 mL) was added NaH (2.0 g, 50.0 mmol, 60 wt % in mineral oil) at 0 [symbol] . The reaction mixture was stirred at 80 [symbol] for 12 h. The reaction mixture was poured into water (200 mL) and extracted with ethyl acetate (50 mL×3). The combined organic layers were washed with brine (150 mL), dried over Na₂SO₄, filtered, and concentrated. The residue was purified by prep-HPLC to provide the desired product (1.40 g, 30% yield) as a white solid. ¹HNMR (400 MHz, DMSO-d₆) δ 8.37 (s, 2H), 7.59 (d, J=9.6 Hz, 1H), 7.51-7.29 (m, 5H), 5.28 (s, 2H).

### Step 2. Synthesis of tert-butyl (5-((3-((6-(benzyloxy)pyridazin-3-yl)carbamoyl)-6-fluoropyridin-2-yl)amino)pentyl)carbamate

To a solution of 2-[5-(tert-butoxycarbonylamino)pentylamino]-6-fluoro-pyridine-3-carboxylic acid (1.7 g, 4.97 mmol), DIEA (2.04 g, 15.7 mmol) and HATU (2.28 g, 6.0 mmol) in DMF (10 mL) was added 6-benzyloxypyridazin-3-amine (1.0 g, 4.97 mmol). After the reaction mixture was stirred at rt for 12 h, it was poured into water (50 mL) and extracted with ethyl acetate (20 mL×3). The combined organic layers were washed with brine (50 mL), dried over Na₂SO₄, filtered, and concentrated. The residue was purified by column chromatography on silica gel (petroleum ether/ethyl acetate=5:1 to 1:1) to provide the desired product (1.20 g, 46% yield) as a yellow oil. ¹HNMR (400 MHz, CDCl₃) δ 8.51 (s, 1H), 8.41 (d, J=10.0 Hz, 2H), 8.06 (t, J=8.0 Hz, 1H), 7.55-7.47 (m, 2H), 7.44-7.31 (m, 3H), 7.13 (d, J=9.6 Hz, 1H), 6.18-6.07 (m, 1H), 5.52 (s, 2H), 3.57-3.44 (m, 2H), 3.15-3.12 (m, 2H), 1.70-1.63 (m, 4H), 1.57-1.52 (m, 2H), 1.44 (s, 9H).

### Step 3. Synthesis of tert-butyl (5-((6-fluoro-3-((6-hydroxypyridazin-3-yl)carbamoyl)pyridin-2-yl)amino)pentyl)carbamate

To a solution of tert-butyl N-[5-[[3-[(6-benzyloxypyridazin-3-yl)carbamoyl]-6-fluoro-2-pyridyl]amino]pentyl]carbamate (300 mg, 0.57 mmol) in MeOH (1 mL) was added 10% Pd/C (200 mg).

The reaction mixture was stirred at rt under hydrogen balloon for 12 h. Then the reaction mixture was filtered through a Celite pad and the filtrate was evaporated under reduced pressure to provide the desired product (350 mg, crude, 98% yield) as a yellow oil. MS (ESI) m/z=435.3 [M+H]⁺.

### Step 4. Synthesis of tert-butyl (5-((6-(methylamino)-3-((6-oxo-1,6-dihydropyridazin-3-yl)carbamoyl)pyridin-2-yl)amino)pentyl)carbamate

To a solution of tert-butyl N-[5-[[6-fluoro-3-[(6-hydroxypyridazin-3-yl)carbamoyl]-2-pyridyl]amino]pentyl]carbamate (350 mg, 0.57 mmol) in EtOH (2 mL) was added TEA (363 mg, 3.59 mmol) and methanamine hydrochloride (196 mg, 2.87 mmol). The reaction mixture was stirred at 100° C. for 12 h. After cooling down to rt, the reaction mixture was poured into water (20 mL) and extracted with ethyl acetate (10 mL×3). The combined organic layers were washed with brine (30 mL), dried over Na₂SO₄, filtered, and concentrated. The residue was purified by prep-TLC (petroleum ether/ethyl acetate=0:1) to provide the desired product (40.0 mg, 15% yield) as a brown solid. ¹HNMR (400 MHz, MeOH-d₄) δ 8.02 (d, J=10.0 Hz, 1H), 7.70 (d, J=8.4 Hz, 1H), 6.96 (d, J=10.0 Hz, 1H), 5.72 (d, J=8.8 Hz, 1H), 3.49 (t, J=6.8 Hz, 2H), 3.04 (t, J=6.8 Hz, 3H), 2.91 (s, 3H), 1.70-1.60 (m, 2H), 1.56-1.48 (m, 4H), 1.42 (s, 9H). MS (ESI) m/z=446.1 [M+H]⁺.

The remaining steps were performed according to the standard procedures to provide the desired product (4.2 mg, 23% yield over 2 steps) as a yellow solid. MS (ESI) m/z=833.9 [M+H]⁺.

### Example 132. 2-((6-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)amino)spiro[3.3]heptan-2-yl)amino)-N-(6-(dimethylamino)pyridazin-3-yl)-6-(methylamino)nicotinamide (CPD-78)

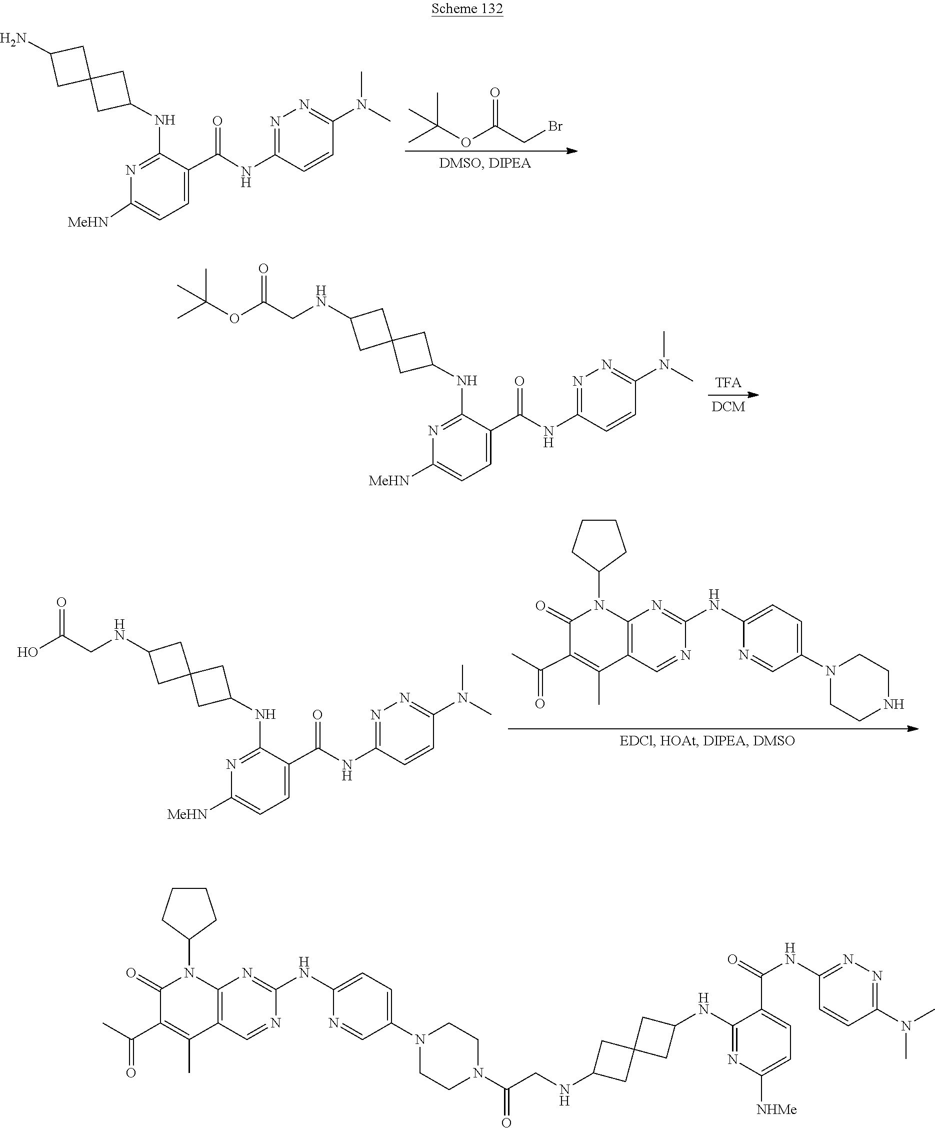

### Step 1. Synthesis of tert-butyl (6-((3-((6-(dimethylamino)pyridazin-3-yl)carbamoyl)-6-(methylamino)pyridin-2-yl)amino)spiro[3.3]heptan-2-yl)glycinate

To a solution of 2-((6-aminospiro[3.3]heptan-2-yl)amino)-N-(6-(dimethylamino)pyridazin-3-yl)-6-(methylamino)nicotinamide (15 mg, 0.03 mmol) and tert-butyl 2-bromoacetate (7.4 mg, 0.03 mmol) in DMSO (2 mL) was added DIPEA (12 mg, 0.09 mmol) at rt. After the reaction mixture was stirred at rt for 1 h, it was purified by reverse-phase chromatography to provide the desired product (5.2 mg, 27% yield) as a white solid. MS (ESI) m/z=511.6 [M+H]⁺.

The remaining steps were performed according to the standard procedures to provide the desired product (2.8 mg, 25% yield over 2 steps) as a yellow solid. MS (ESI) m/z=884.8 [M+H]⁺.

### Example 133. 2-((3-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-(methylamino)nicotinamide (CPD-79)

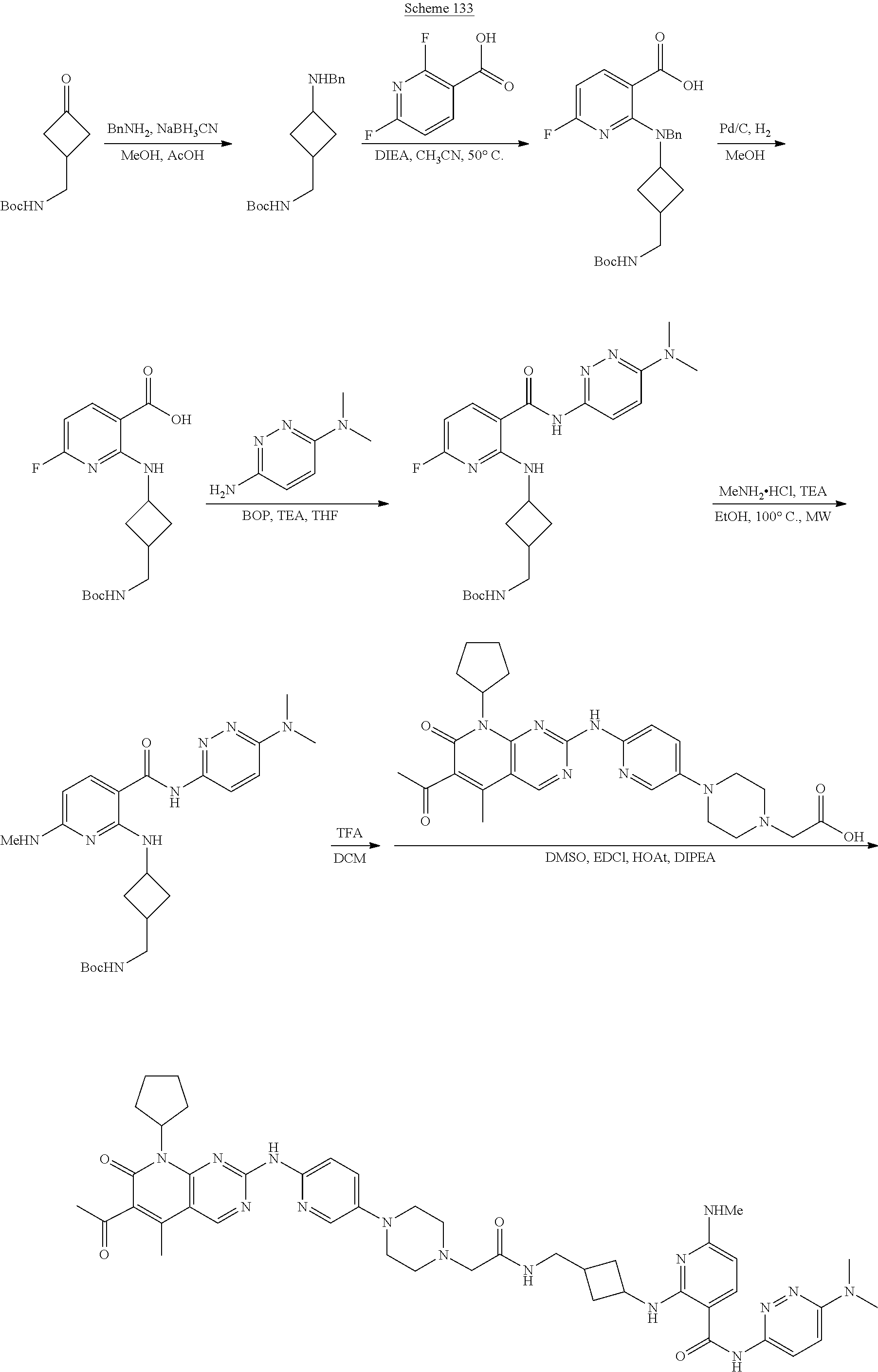

### Step 1. Synthesis of tert-butyl N-[[3-(benzylamino)cyclobutyl]methyl]carbamate

To a mixture of tert-butyl N-[(3-oxocyclobutyl)methyl]carbamate (0.50 g, 2.51 mmol) and phenylmethanamine (649 mg, 6.05 mmol) in MeOH (5 mL) were added NaBH₃CN (250 mg, 3.98 mmol) and AcOH (252 mg, 4.20 mmol) at rt. After the reaction mixture was stirred at rt for 12 h, it was poured into water (20 mL) and extracted with ethyl acetate (30 mL×3). The combined organic phase was washed with brine (50 mL), dried with anhydrous Na₂SO₄, filtered, and concentrated in vacuum. The residue was purified by prep-TLC (dichloromethane/methanol=10:1) to provide the desired product (0.55 g, crude) as a yellow oil. ¹HNMR (400 MHz, CDCl₃) δ 7.35-7.30 (m, 4H), 7.27-7.20 (m, 1H), 3.74-3.68 (m, 2H), 3.23-3.10 (m, 3H), 2.39-2.30 (m, 2H), 2.20-1.97 (m, 2H), 1.95-1.82 (m, 1H), 1.45 (s, 9H).

### Step 2. Synthesis of 2-[benzyl-[3-[(tert-butoxycarbonylamino)methyl]cyclobutyl]amino]-6-fluoro-pyridine-3-carboxylic acid

To a mixture of 2,6-difluoropyridine-3-carboxylic acid (260 mg, 1.63 mmol) and tert-butyl N-[[3-(benzylamino)cyclobutyl]methyl]carbamate (0.35 g, 1.21 mmol) in CH₃CN (5 mL) was added DIEA (519 mg, 4.02 mmol) at rt. After the reaction mixture was stirred at 50 [symbol] for 12 h, it was concentrated and purified by prep-TLC (dichloromethane/methanol=10:1) to provide the desired product (0.50 g, 96% yield) as a yellow oil. MS (ESI) m/z=430.3 [M+H]⁺.

### Step 3. Synthesis of 2-[[3-[(tert-butoxycarbonylamino)methyl]cyclobutyl]amino]-6-fluoro-pyridine-3-carboxylic acid

To a solution of 2-[benzyl-[3-[(tert-butoxycarbonylamino)methyl]cyclobutyl]amino]-6-fluoro-pyridine-3-carboxylic acid (0.50 g, 1.16 mmol) in MeOH (5 mL) was added 10% Pd/C (0.05 g) at rt under H₂. The reaction mixture was stirred at rt for 12 h. Then the reaction mixture was filtered through a Celite pad and the filtrate was evaporated in vacuo to provide the desired product (0.36 g, 91% yield) as a gray oil. ¹HNMR (400 MHz, CDCl₃) δ 8.29-8.12 (m, 1H), 7.42-7.30 (m, 1H), 6.04-5.91 (m, 1H), 3.74-3.64 (m, 1H), 3.16-3.04 (m, 3H), 2.26-2.00 (m, 4H), 1.42 (s, 9H).

The remaining steps were performed according to the procedures for preparing CPD-34 to provide the desired product (3.9 mg, 2% yield over 4 steps) as a yellow solid. MS (ESI) m/z=858.8 [M+H]⁺.

### Example 134. 2-((3-((3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propanamido)methyl)cyclobutyl)amino)-N-(6-(dimethylamino)pyridazin-3-yl)-6-(methylamino)nicotinamide (CPD-80)

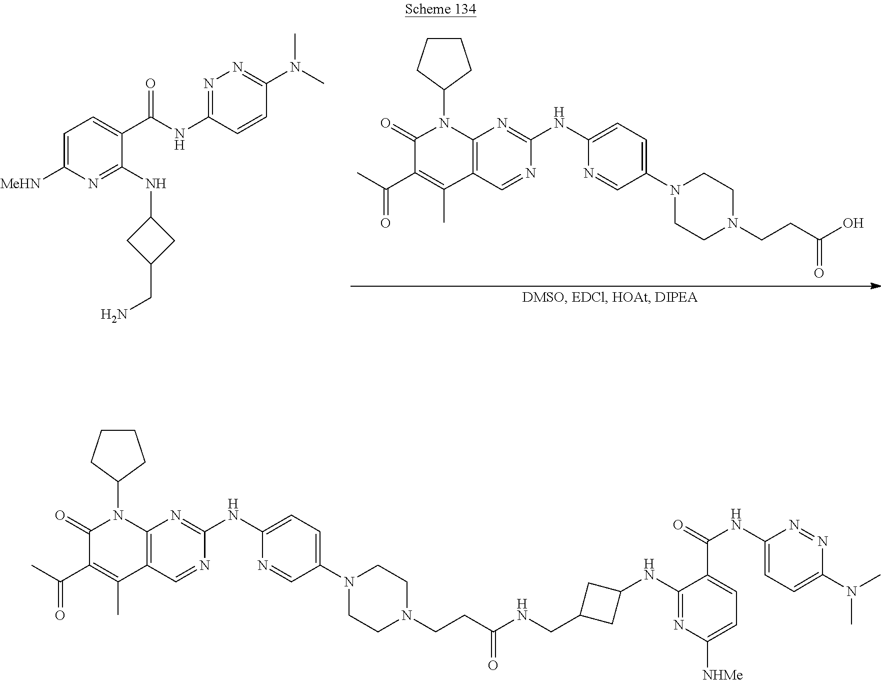

CPD-80 was synthesized following the procedures for preparing CPD-1 (8.7 mg, 47% yield) as a yellow solid. MS (ESI) m/z=872.9 [M+H]⁺.

### Example 135. 4-(6-((6-Acetyl-8-cyclopentyl-5-methyl-7-oxo-7,8-dihydropyrido[2,3-d]pyrimidin-2-yl)amino)pyridin-3-yl)-N-((6-((3-((6-(dimethylamino)pyridazin-3-yl)carbamoyl)-6-(methylamino)pyridin-2-yl)amino)spiro[3.3]heptan-2-yl)methyl)piperazine-1-carboxamide (CPD-81)

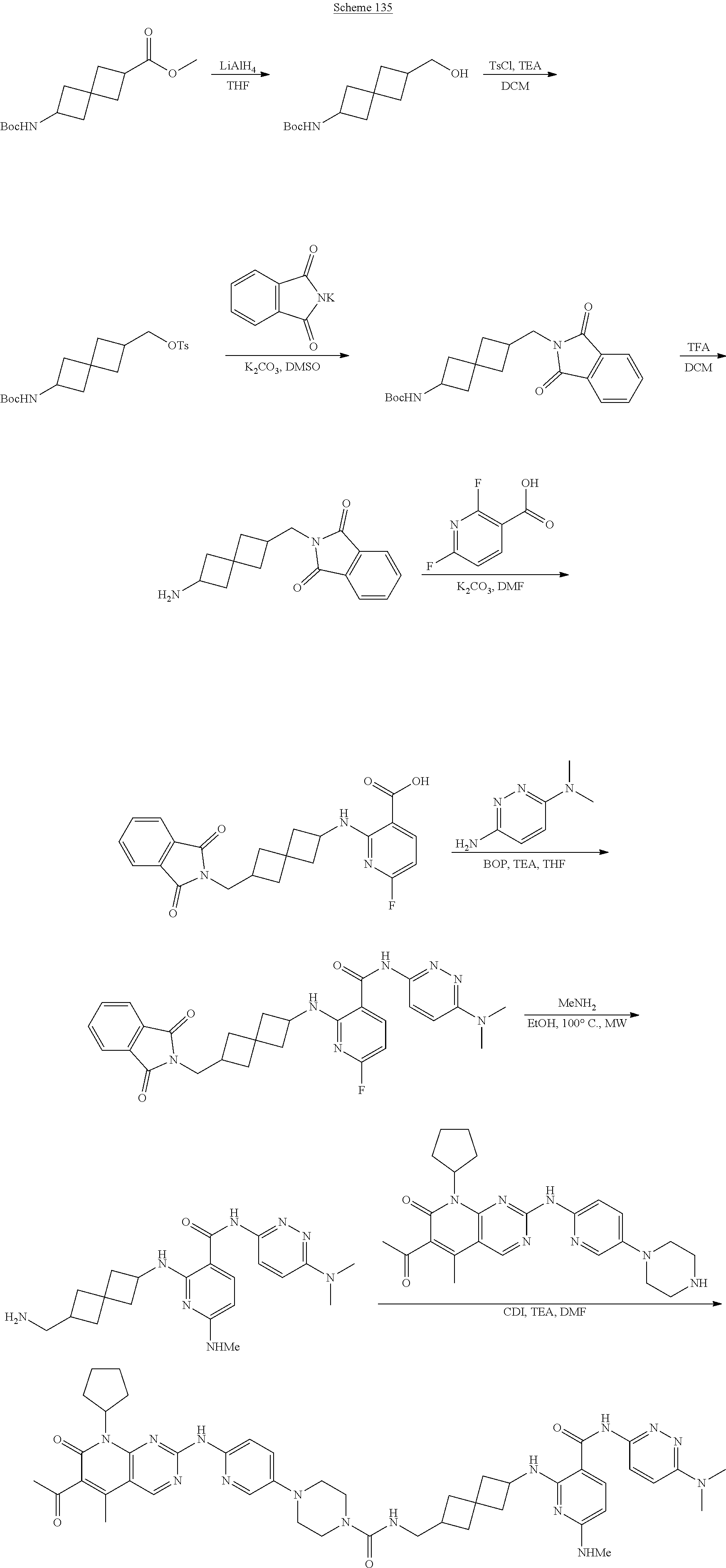

### Step 1. Synthesis of tert-butyl (6-(hydroxymethyl)spiro[3.3]heptan-2-yl)carbamate

To a solution of methyl 6-((tert-butoxycarbonyl)amino)spiro[3.3]heptane-2-carboxylate (800 mg, 2.97 mmol) in THF (50 mL) was added LiAlH₄ (225 mg, 5.94 mmol) in portions at 0° C. After the reaction mixture was stirred at rt for 2 h, it was quenched with water (20 mL) slowly and extracted with ethyl acetate (3×50 mL). The combined organic phase was washed with brine, dried with anhydrous Na₂SO₄, filtered, and concentrated. The residue was purified by silica gel chromatography to provide the desired product (600 mg, 84% yield) as a colorless oil.

### Step 2. Synthesis of (6-((tert-butoxycarbonyl)amino)spiro[3.3]heptan-2-yl)methyl 4-methylbenzenesulfonate

To a solution of tert-butyl (6-(hydroxymethyl)spiro[3.3]heptan-2-yl)carbamate (200 mg, 0.83 mmol) in DCM (30 mL) were added 4-methylbenzenesulfonyl chloride (158 mg, 0.83 mmol) and TEA (840 mg, 8.29 mmol). After the reaction mixture was stirred at 40° C. for 4 h, it was poured into water (30 mL) and extracted with DCM (3×30 mL). The combined organic layers were washed with saturated brine (20 mL), dried over anhydrous sodium sulfate, filtered, and evaporated under reduced pressure. The resulting residue was purified by silica gel chromatography to provide the desired product (200 mg, 61% yield) as a colorless oil. MS (ESI) m/z=296.3 [M+H-100]⁺.

### Step 3. Synthesis of tert-butyl (6-((1,3-dioxoisoindolin-2-yl)methyl)spiro[3.3]heptan-2-yl)carbamate

To a solution of (6-((tert-butoxycarbonyl)amino)spiro[3.3]heptan-2-yl)methyl 4-methylbenzenesulfonate (200 mg, 0.51 mmol) in DMSO (10 mL) were added potassium 1,3-dioxoisoindolin-2-ide (140 mg, 0.76 mmol) and K₂CO₃ (350 mg, 2.53 mmol) at rt. The mixture was stirred 90° C. for 6 h. After cooling down to rt, the reaction mixture was poured into water (50 mL) and extracted with ethyl acetate (3×50 mL). The combined organic layers were washed with saturated brine (30 mL), dried over anhydrous sodium sulfate, filtered, and evaporated under reduced pressure. The resulting residue was purified by silica gel chromatography to provide the desired product (150 mg, 80% yield) as a light-yellow solid. MS (ESI) m/z=271.3 [M+H-100]⁺.

### Step 4. Synthesis of 2-((6-aminospiro[3.3]heptan-2-yl)methyl)isoindoline-1,3-dione

To a solution of tert-butyl (6-((1,3-dioxoisoindolin-2-yl)methyl)spiro[3.3]heptan-2-yl)carbamate (150 mg, 0.40 mmol) in DCM (5 mL) was added TFA (2 mL). The mixture was stirred at rt for 1 h. Then the solution was concentrated under reduced pressure to provide the crude product (100 mg, 91% yield) as a brown solid. This compound was used directly in the next step without further purification. MS (ESI) m/z=271.4 [M+H]⁺.

### Step 5. Synthesis of 2-((6-((1,3-dioxoisoindolin-2-yl)methyl)spiro[3.3]heptan-2-yl)amino)-6-fluoronicotinic acid

To a solution of 2-((6-aminospiro[3.3]heptan-2-yl)methyl)isoindoline-1,3-dione (100 mg, 0.37 mmol) in DMF (5 mL) were added 2,6-difluoronicotinic acid (60 mg, 0.37 mmol) and K₂CO₃ (255 mg, 1.85 mmol) at rt. The reaction mixture was stirred 90° C. for 6 h. After cooling down to rt, the solution was diluted with water (30 mL) and the pH was adjusted to 5~6 with HCl solution (1 M). The aqueous phase was extracted with EtOAc (3×40 mL). The combined organic layers were washed with saturated brine (20 mL), dried over anhydrous sodium sulfate, filtered, and evaporated under reduced pressure. The resulting residue was purified by reverse-phase chromatography to provide the desired product (70 mg, 46% yield) as a light-yellow solid. MS (ESI) m/z=410.4 [M+H]⁺.

### Step 6. Synthesis of N-(6-(dimethylamino)pyridazin-3-yl)-2-((6-((1,3-dioxoisoindolin-2-yl)methyl)spiro[3.3]heptan-2-yl)amino)-6-fluoronicotinamide

To a solution of 2-((6-((1,3-dioxoisoindolin-2-yl)methyl)spiro[3.3]heptan-2-yl)amino)-6-fluoronicotinic acid (70 mg, 0.17 mmol) in THF (10 mL) were added N³,N³-dimethylpyridazine-3,6-diamine (23 mg, 0.17 mmol), BOP (150 mg, 0.34 mmol) and TEA (86 mg, 0.85 mmol). After the reaction mixture was stirred at rt for 2 h, it was quenched with water (20 mL) and extracted with ethyl acetate (2×30 mL). The combined organic layers were washed with saturated brine (20 mL), dried over anhydrous sodium sulfate, filtered, and evaporated under reduced pressure. The resulting residue was purified by silica gel chromatography to provide the desired product (25 mg, 27% yield) as a brown solid. MS (ESI) m/z=530.5 [M+H]⁺

### Step 7. Synthesis of 2-((6-(aminomethyl)spiro[3.3]heptan-2-yl)amino)-N-(6-(dimethylamino)pyridazin-3-yl)-6-(methylamino)nicotinamide

To a solution of N-(6-(dimethylamino)pyridazin-3-yl)-2-((6-((1,3-dioxoisoindolin-2-yl)methyl)spiro[3.3]heptan-2-yl)amino)-6-fluoronicotinamide (20 mg, 0.038 mmol) in EtOH (2 mL) was added MeNH₂ solution (30% in EtOH, 2 mL). The reaction mixture was stirred at 100° C. under microwave irradiation for 1 h. After cooling down to rt, the reaction mixture was poured into water (30 mL) and extracted with ethyl acetate (3×30 mL). The combined organic layers were washed with saturated brine (20 mL), dried over anhydrous sodium sulfate, filtered, and evaporated under reduced pressure. The resulting residue was purified by reverse-phase chromatography to provide the desired product (12 mg, 77% yield) as a brown solid. MS (ESI) m/z=411.5 [M+H]⁺.

### Step 8. Synthesis of 4-(6-((6-acetyl-8-cyclopentyl-5-methyl-7-oxo-7,8-dihydropyrido[2,3-d]pyrimidin-2-yl)amino)pyridin-3-yl)-N-((6-((3-((6-(dimethylamino)pyridazin-3-yl)carbamoyl)-6-(methylamino)pyridin-2-yl)amino)spiro[3.3]heptan-2-yl)methyl)piperazine-1-carboxamide

To a solution of 2-((6-(aminomethyl)spiro[3.3]heptan-2-yl)amino)-N-(6-(dimethylamino)pyridazin-3-yl)-6-(methylamino)nicotinamide (12 mg, 0.029 mmol) in DMF (3 mL) were added CDI (7 mg, 0.044 mmol) and TEA (30 mg, 0.29 mmol). The mixture was stirred at rt for 20 min, then 6-acetyl-8-cyclopentyl-5-methyl-2-((5-(piperazin-1-yl)pyridin-2-yl)amino)pyrido[2,3-d]pyrimidin-7(8H)-one (20 mg, 0.044 mmol) was added. The reaction mixture was stirred at rt for additional 12 h, before it was poured into water (20 mL) and extracted with ethyl acetate (3×30 mL). The combined organic layers were washed with saturated brine (20 mL), dried over anhydrous sodium sulfate, filtered, and evaporated under reduced pressure. The resulting residue was purified by reverse-phase chromatography to give the desired product (5 mg, 19% yield) as a yellow solid. MS (ESI) m/z=884.9 [M+H]⁺.

### Example 136. 2-((6-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)spiro[3.3]heptan-2-yl)amino)-N-(6-(dimethylamino)pyridazin-3-yl)-6-(methylamino)nicotinamide (CPD-82)

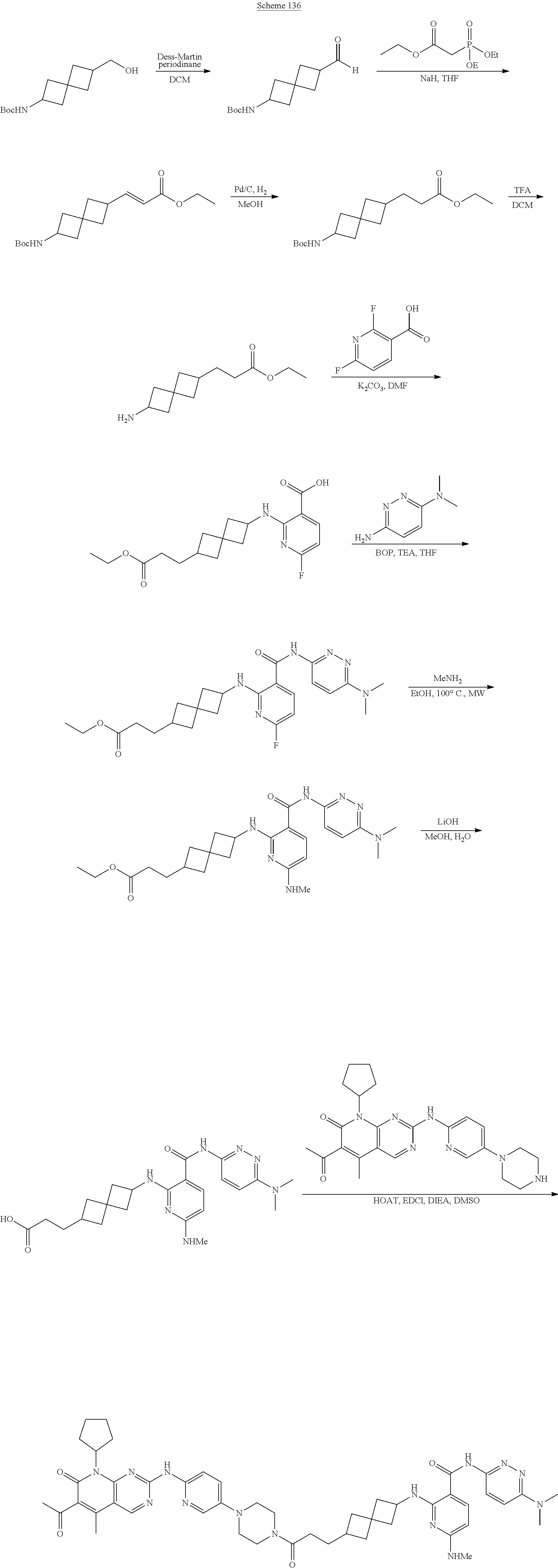

### Step 1. Synthesis of tert-butyl (6-formylspiro[3.3]heptan-2-yl)carbamate

To a solution of tert-butyl (6-(hydroxymethyl)spiro[3.3]heptan-2-yl)carbamate (400 mg, 1.66 mmol) in DCM (20 mL) was added Dess-Martin periodinane (1050 mg, 2.49 mmol) in portions at rt. After the reaction mixture was stirred at rt for 10 h, it was quenched with water (20 mL) and extracted with DCM (3×30 mL). The combined organic phase was washed with brine, dried over anhydrous sodium sulfate, filtered, and concentrated. The residue was purified by silica gel chromatography to provide the desired product (350 mg, 88% yield) as a colorless oil.

### Step 2. Synthesis of methyl (E)-3-(6-((tert-butoxycarbonyl)amino)spiro[3.3]heptan-2-yl)acrylate

To a solution of tert-butyl (6-formylspiro[3.3]heptan-2-yl)carbamate (350 mg, 1.46 mmol) in THF (25 mL) was added NaH (117 mg, 2.93 mmol, 60% in mineral oil) in portions. After the mixture was stirred at 0° C. for 1 h, ethyl 2-(diethoxyphosphoryl)acetate (330 mg, 1.46 mmol) was added. The mixture was warmed to rt and stirred for another 1 h. The mixture was quenched with water (20 mL) and extracted with ethyl acetate (3×50 mL). The organic phase was washed with brine, dried over anhydrous sodium sulfate, filtered, and concentrated. The residue was purified by silica gel chromatography to provide the desired product (400 mg, 88% yield) as a colorless oil.

### Step 3. Synthesis of ethyl 3-(6-((tert-butoxycarbonyl)amino)spiro[3.3]heptan-2-yl)propanoate

To a solution of ethyl (E)-3-(6-((tert-butoxycarbonyl)amino)spiro[3.3]heptan-2-yl)acrylate (400 mg, 1.29 mmol) in MeOH (20 mL) was added 10% Pd/C (100 mg). The reaction mixture was stirred at rt under hydrogen atmosphere for 5 h. The mixture was filtered through celite and the filtrate was concentrated under reduced pressure to provide the desired product (380 mg, 94% yield) as a brown oil. This compound was used directly in the next step without further purification.

### Step 4 to 7. Synthesis of ethyl 3-(6-((3-((6-(dimethylamino)pyridazin-3-yl)carbamoyl)-6-(methylamino)pyridin-2-yl)amino)spiro[3.3]heptan-2-yl)propanoate

These four steps were performed according to the procedures of step 4 to 7 for preparing CPD-81 to provide the title compound (200 mg, 30% yield over 4 steps) as a yellow solid. MS (ESI) m/z=482.5 [M+H]⁺.

### Step 8. Synthesis of 3-(6-((3-((6-(dimethylamino)pyridazin-3-yl)carbamoyl)-6-(methylamino)pyridin-2-yl)amino)spiro[3.3]heptan-2-yl)propanoic acid

To a solution of ethyl 3-(6-((3-((6-(dimethylamino)pyridazin-3-yl)carbamoyl)-6-(methylamino)pyridin-2-yl)amino)spiro[3.3]heptan-2-yl)propanoate (25 mg, 0.052 mmol) in MeOH (8 mL) and water (2 mL) was added LiOH (13 mg, 0.52 mmol). The mixture was stirred at 50° C. for 8 h. After cooling down to rt, the solution was diluted with water (30 mL) and the pH was adjusted to 5~6 with HCl solution (1 M). The solution was extracted with EtOAc (3×30 mL). The combined organic layers were washed with saturated brine (20 mL), dried over anhydrous sodium sulfate, filtered, and evaporated under reduced pressure. The resulting residue was purified by reverse-phase chromatography to give the desired product (15 mg, 64% yield) as a light-yellow solid. MS (ESI) m/z=454.5 [M+H]⁺.

### Step 9. Synthesis of 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)spiro[3.3]heptan-2-yl)amino)-N-(6-(dimethylamino)pyridazin-3-yl)-6-(methylamino)nicotinamide

The title compound was synthesized following the standard procedure (5 mg, 17% yield) as a yellow solid. MS (ESI) m/z=883.8 [M+H]⁺

### Example 137. 2-((6-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propyl)spiro[3.3]heptan-2-yl)amino)-N-(6-(dimethylamino)pyridazin-3-yl)-6-(methylamino)nicotinamide (CPD-83)

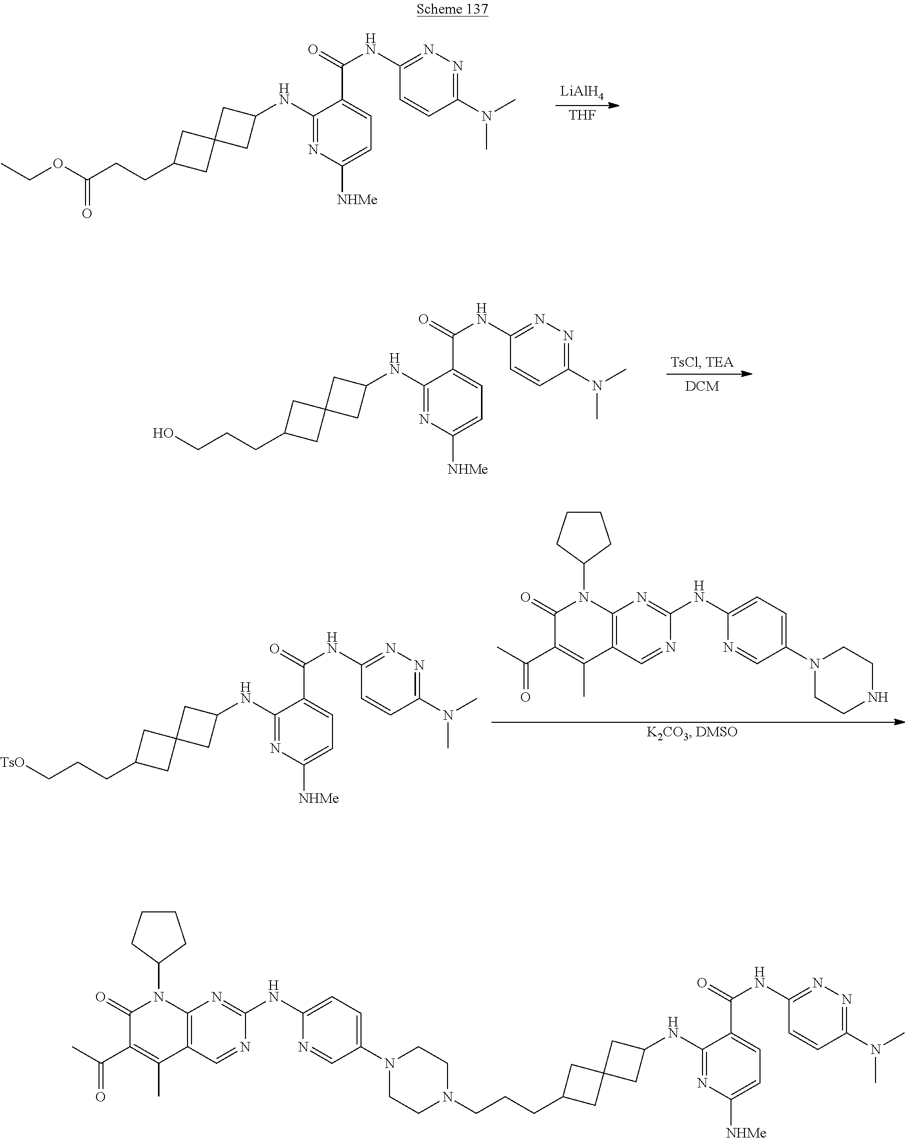

CPD-83 was synthesized following the procedures of step 1 to 3 for preparing CPD-81 (3.0 mg, 4% yield over 3 steps) as a yellow solid. MS (ESI) m/z=869.8 [M+H]⁺.

### Example 138. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)spiro[3.3]heptan-2-yl)amino)-N-(6-(dimethylamino)pyridazin-3-yl)-6-(methylamino)nicotinamide (CPD-84)

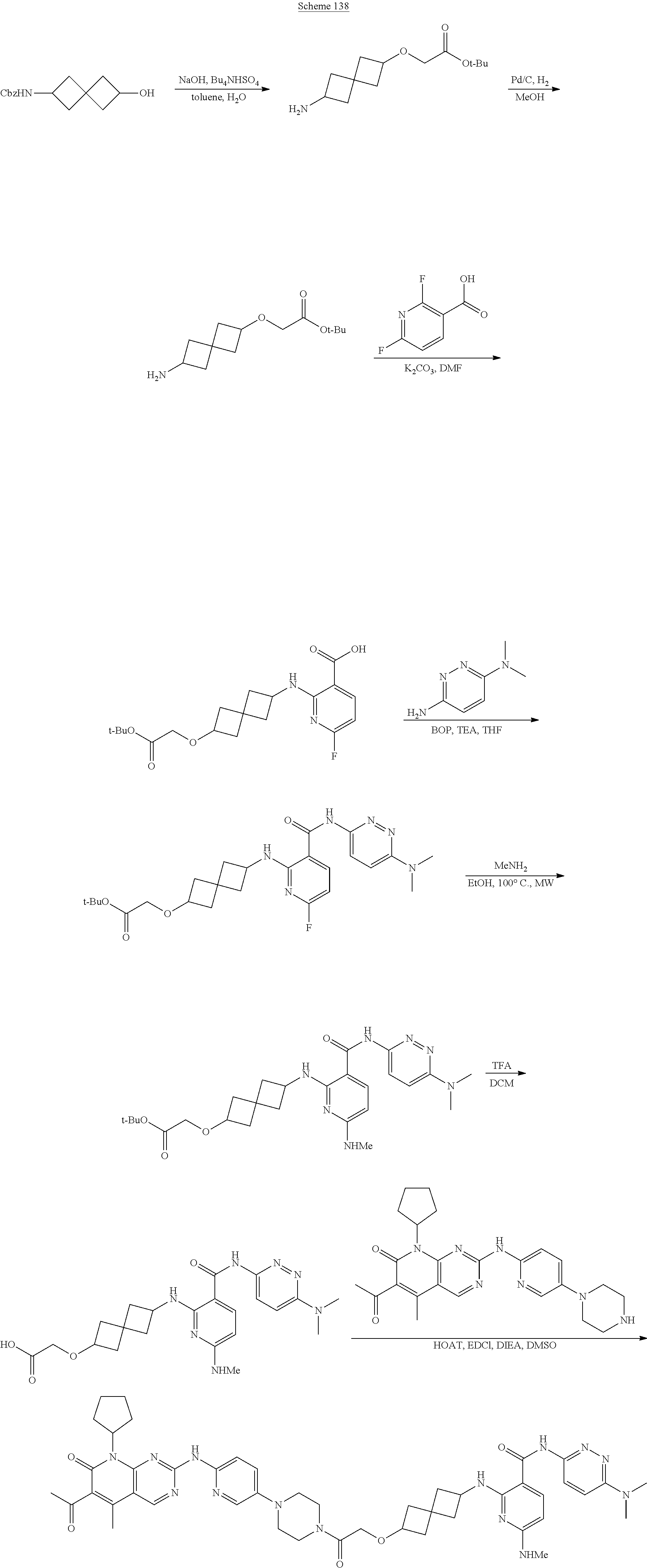

### Step 1. Synthesis of tert-butyl 2-((6-(((benzyloxy)carbonyl)amino)spiro[3.3]heptan-2-yl)oxy)acetate

To a solution of benzyl (6-hydroxyspiro[3.3]heptan-2-yl)carbamate (200 mg, 0.77 mmol) and tert-butyl 2-bromoacetate (179 mg, 0.92 mmol) in toluene (15 mL) and water (5 mL) were added tetrabutylammonium hydrogen sulfate (208 mg, 0.62 mmol) and sodium hydroxide (2.5 g, 62.5 mmol) at rt. After the reaction mixture was stirred at rt for 12 h, it was quenched with H₂O (20 mL) and extracted with ethyl acetate (20 mL×3). The organic phase was washed with brine, dried over anhydrous Na₂SO₄, filtered, and concentrated. The residue was purified by silica gel column chromatography (petroleum ether/ethyl acetate - 1:1) to provide the desired product (100 mg, 35% yield) as a colorless oil. MS (ESI) m/z=320.3 [M+H-56]⁺.

### Step 2. Synthesis of tert-butyl 2-((6-aminospiro[3.3]heptan-2-yl)oxy)acetate

To a solution of tert-butyl 2-[6-(benzyloxycarbonylamino)spiro[3.3]heptan-2-yl]oxyacetate (40 mg, 0.11 mmol) in methanol (5 mL) was added 10% Pd/C (13 mg). The reaction mixture was stirred at rt under hydrogen atmosphere for 12 h. Then the mixture was filtered through Celite and the filtrate was concentrated under reduced pressure to provide the desired product (25 mg, crude) as a yellow oil. MS (ESI) m/z 242.3 [M+H]⁺.

The remaining steps were performed according to the procedures of steps 5 to 7 for preparing CPD-81 and steps 6 to 7 for preparing CPD-25 to provide the desired product (2.5 mg, 2% yield over 5 steps) as a yellow solid. MS (ESI) m/z=885.8 [M+H]⁺.

### Example 139. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(tert-butylamino)-N-(6-(dimethylamino)pyridazin-3-yl)nicotinamide (CPD-85)

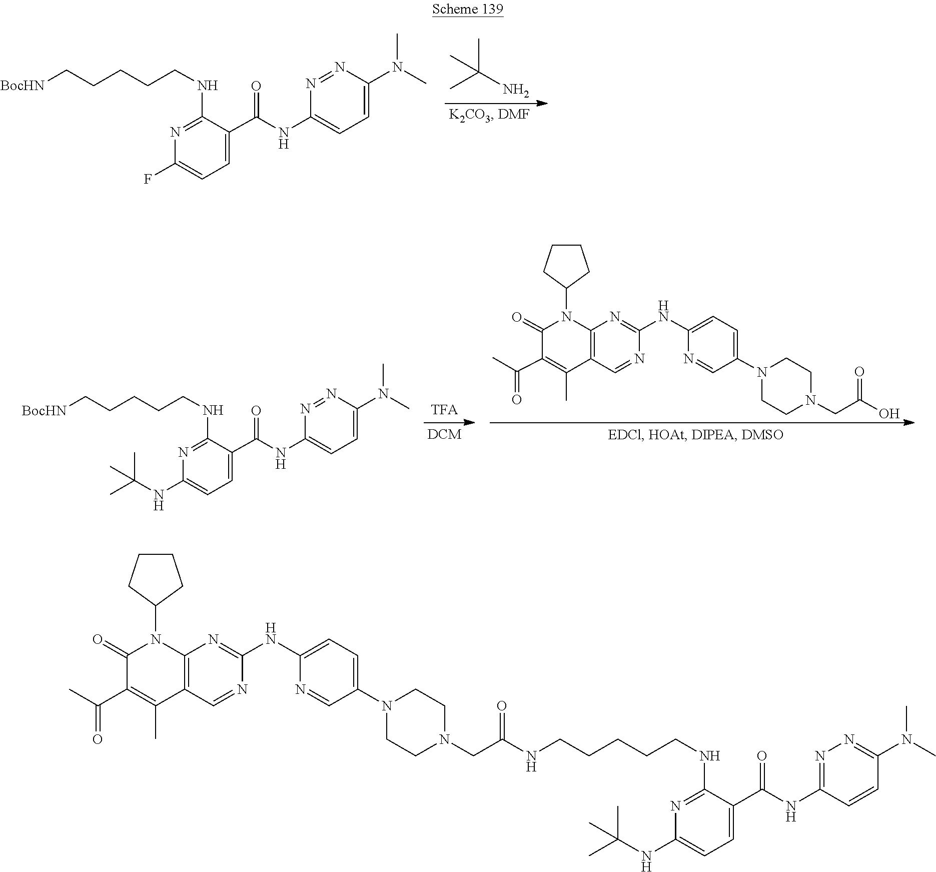

CPD-85 was synthesized following the procedures for preparing CPD-59 (0.73 mg, 1% yield over 3 steps) as a yellow solid. MS (ESI) m/z=902.8 [M+H].

### Example 140. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropylpyridin-4-yl)-6-(methylamino)nicotinamide (CPD-86)

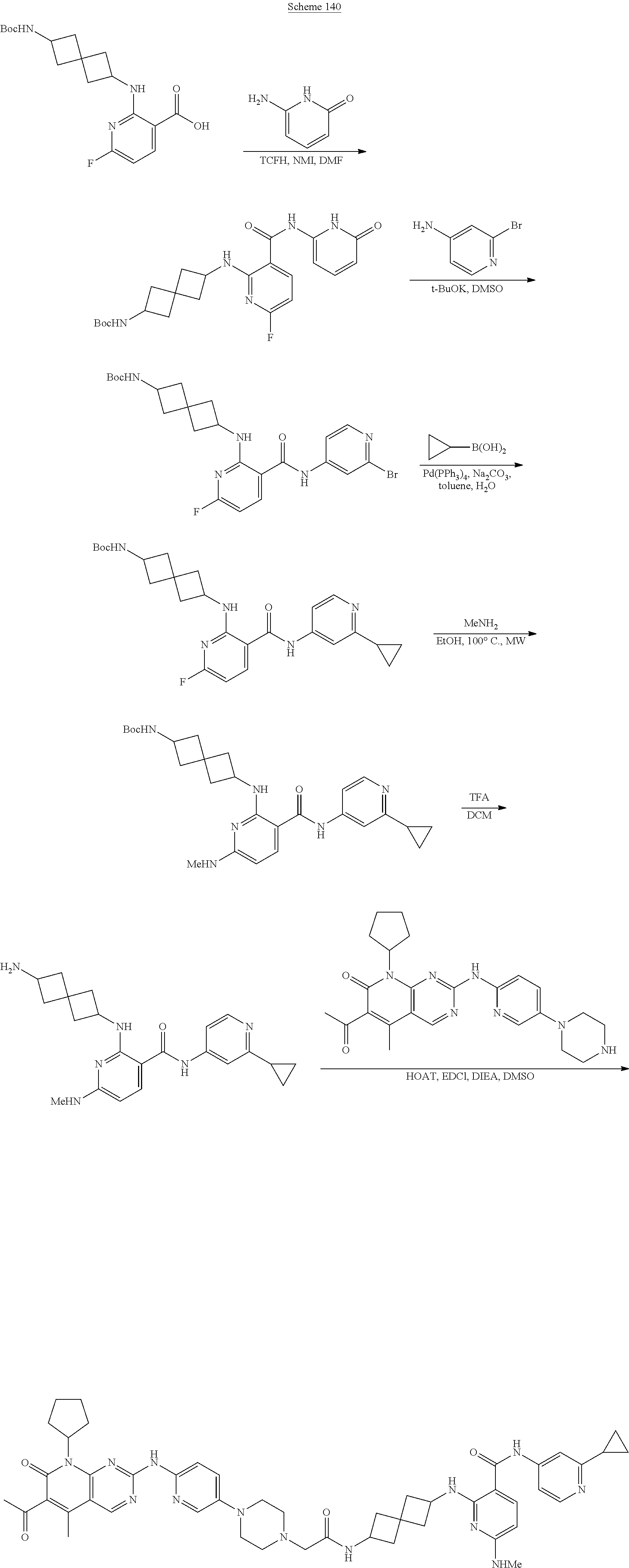

### Step 1. Synthesis of tert-butyl (6-((6-fluoro-3-((6-oxo-1,6-dihydropyridin-2-yl)carbamoyl)pyridin-2-yl)amino)spiro[3.3]heptan-2-yl)carbamate

A mixture of 2-[[2-(tert-butoxycarbonylamino)spiro[3.3]heptan-6-yl]amino]-6-fluoro-pyridine-3-carboxylic acid (130 mg, 0.36 mmol), 6-amino-1H-pyridin-2-one (58.8 mg, 0.53 mmol), NMI (116.8 mg, 1.42 mmol) and TCFH (149.7 mg, 0.53 mmol) in DMF (2 mL) was stirred at rt for 2 h. The reaction mixture was diluted with EtOAc (20 mL) and washed with brine (10 mL×3). The organic layer was concentrated and purified by reverse- phase chromatography to provide the desired product (140 mg, 86% yield) as a white solid. MS (ESI) m/z=458.8 [M+H]⁺.

### Step 2. Synthesis of tert-butyl (6-((3-((2-Bromopyridin-4-yl)carbamoyl)-6-fluoropyridin-2-yl)amino)spiro[3.3]heptan-2-yl)carbamate

To a mixture of tert-butyl (6-((6-fluoro-3-((6-oxo-1,6-dihydropyridin-2-yl)carbamoyl)pyridin-2-yl)amino)spiro[3.3]heptan-2-yl)carbamate (50 mg, 0.11 mmol) and 2-bromopyridin-4-amine (37.8 mg, 0.22 mmol) in DMSO (1 mL) was added t-BuOK solution (1 M in THF, 0.3 mL, 0.3 mmol) at 0° C. The reaction mixture was stirred at rt for 20 min. Then the reaction mixture was purified by reverse-phase chromatography to provide the desired product (25 mg, 44% yield) as a yellow solid. MS (ESI) m/z 520.4 [M+H]⁺.

### Step 3. Synthesis of tert-butyl (6-((3-((2-cyclopropylpyridin-4-yl)carbamoyl)-6-fluoropyridin-2-yl)amino)spiro[3.3]heptan-2-yl)carbamate

A mixture of tert-butyl (6-((3-((2-Bromopyridin-4-yl)carbamoyl)-6-fluoropyridin-2-yl)amino)spiro[3.3]heptan-2-yl)carbamate (25 mg, 0.048 mmol), cyclopropylboronic acid (12.4 mg, 0.14 mmol), Na₂CO₃ (25.5 mg, 0.24 mmol) and Pd(PPh₃)₄ (8.3 mg, 0.007 mmol) in toluene (2 mL) and water (0.4 mL) was stirred at 100 [symbol] for 16 h under nitrogen atmosphere. After cooling down to rt, the reaction mixture was diluted with water (5 mL) and extracted with ethyl acetate (10 mL×3). The combined organic layers was washed with brine, dried over anhydrous Na₂SO₄, filtered, and concentrated. The residue was purified by silica gel chromatography to provide the desired product (15 mg, 65% yield) as a yellow solid. MS (ESI) m/z=482.8 [M+H]⁺.

The remaining steps were performed according to the procedures of CDP-25 to provide the desired product (2.7 mg, 10% yield over 3 steps) as a yellow solid. MS (ESI) m/z=880.7 [M+H]⁺.

### Example 141. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-(trifluoromethyl)pyridin-2-yl)-6-(methylamino)nicotinamide (CPD-87)

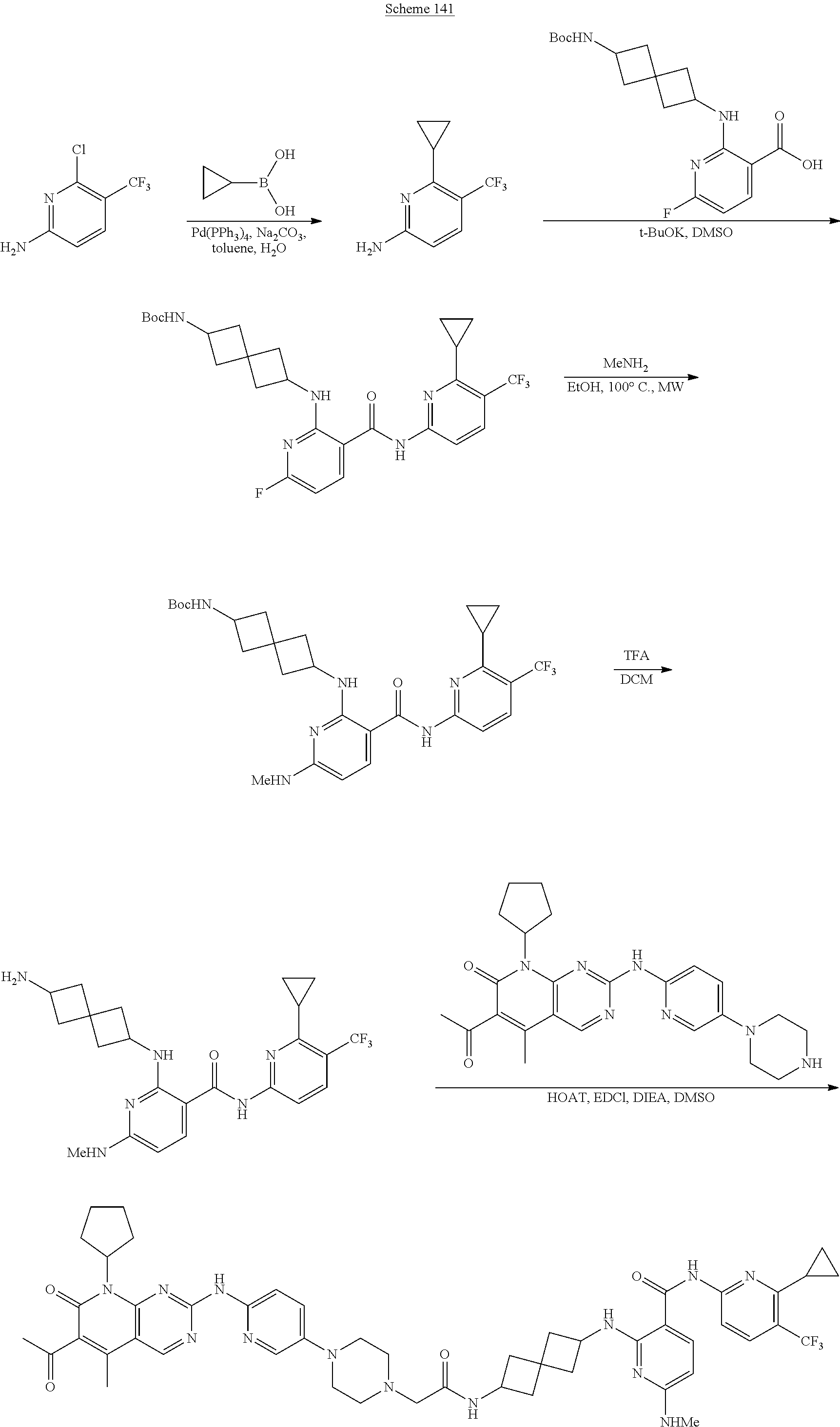

### Step 1. Synthesis of 6-cyclopropyl-5-(trifluoromethyl)pyridin-2-amine

To a solution of 6-chloro-5-(trifluoromethyl)pyridin-2-amine (200 mg, 1.02 mmol) and cyclopropylboronic acid (87.4 mg, 1.02 mmol) in toluene (10 mL) and H₂O (2 mL) were added Na₂CO₃ (107.8 mg, 1.02 mmol) and Pd(PPh₃)₄ (1 17.5 mg, 0. 102 mmol). The reaction mixture was stirred at 90 [symbol] for 18 h. After cooling down to rt, the mixture was quenched with H₂O (20 mL) and extracted with ethyl acetate (20 mL×3). The organic phase was washed with brine, dried over anhydrous Na₂SO₄, filtered, and concentrated. The residue was purified by silica gel column chromatography to afford the desired product (40 mg, 19% yield) as a yellow oil. MS (ESI) m/z=203.2 [M+H]⁺.

The remaining steps were performed according to the procedures of CPD-86 and CPD-25 to provide the desired product (3.3 mg, 4% yield over 4 steps) as a yellow solid. MS (ESI) m/z=948.7 [M+H]⁺.

### Example 142. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridin-2-yl)-6-(methylamino)nicotinamide (CPD-88)

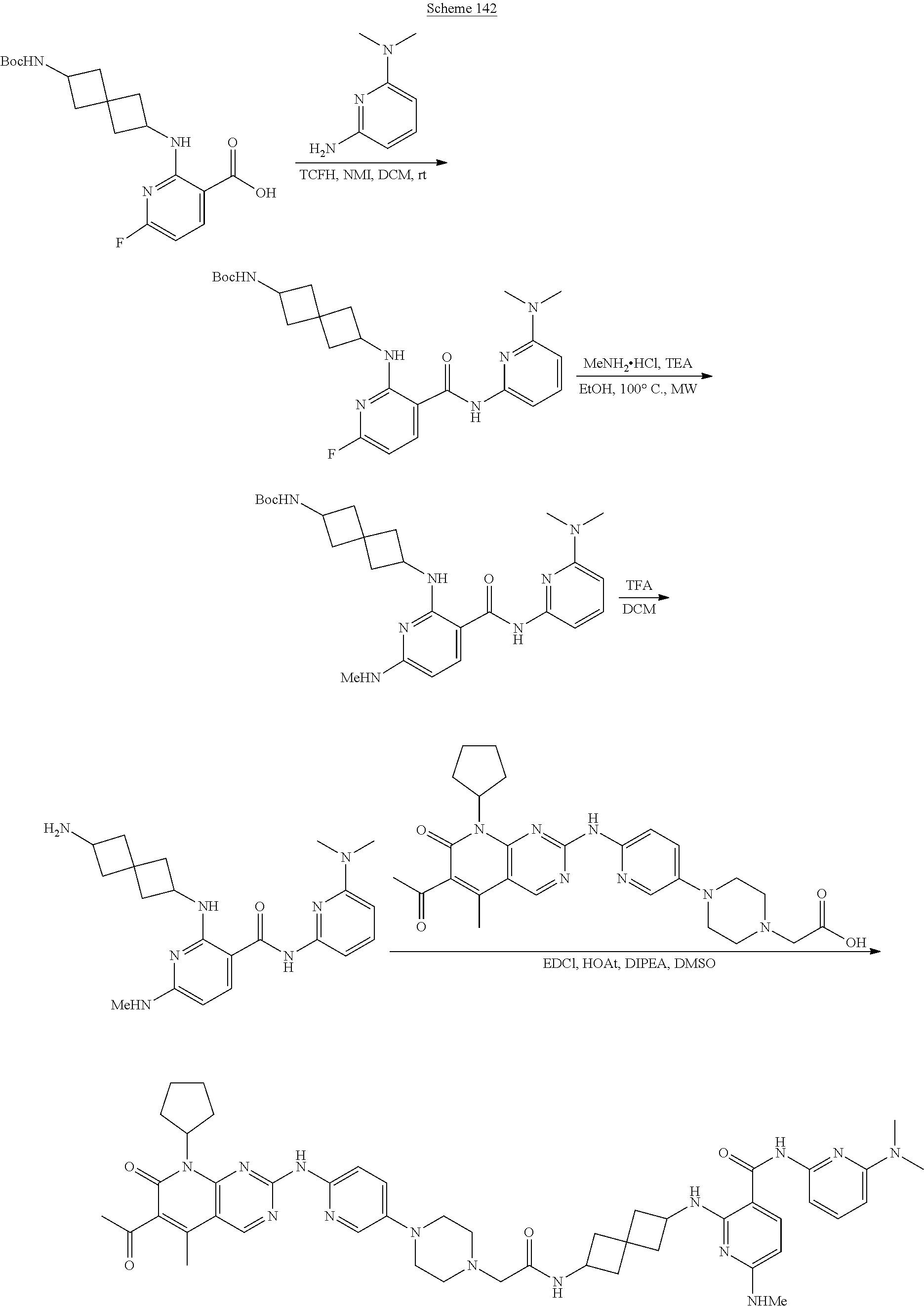

CPD-88 was synthesized following the procedures for preparing CPD-31 (3.2 mg, 4% yield over 4 steps) as a yellow solid. MS (ESI) m/z=883.8 [M+H]⁺.

### Example 143. 2-((2-(1-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)ethyl)amino)-N-(6-(dimethylamino)pyridazin-3-yl)-6-(methylamino)nicotinamide (CPD-89)

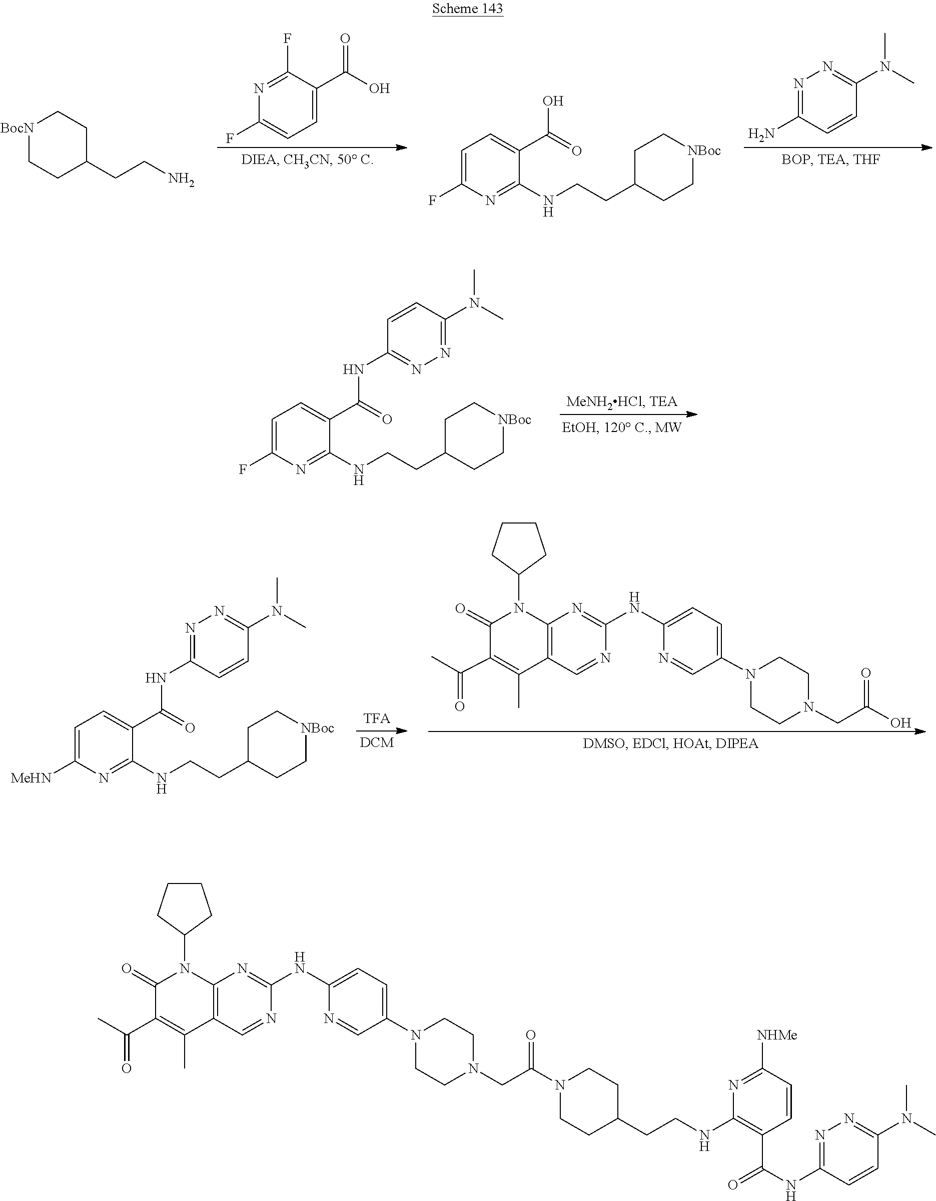

CPD-89 was synthesized following the procedures for preparing CPD-34 (1.2 mg, 1% yield over 5 steps) as a yellow solid. MS (ESI) m/z=886.8 [M+H]⁺.

### Example 144. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyanopyridin-2-yl)-6-(methylanino)nicotinamide (CPD-90)

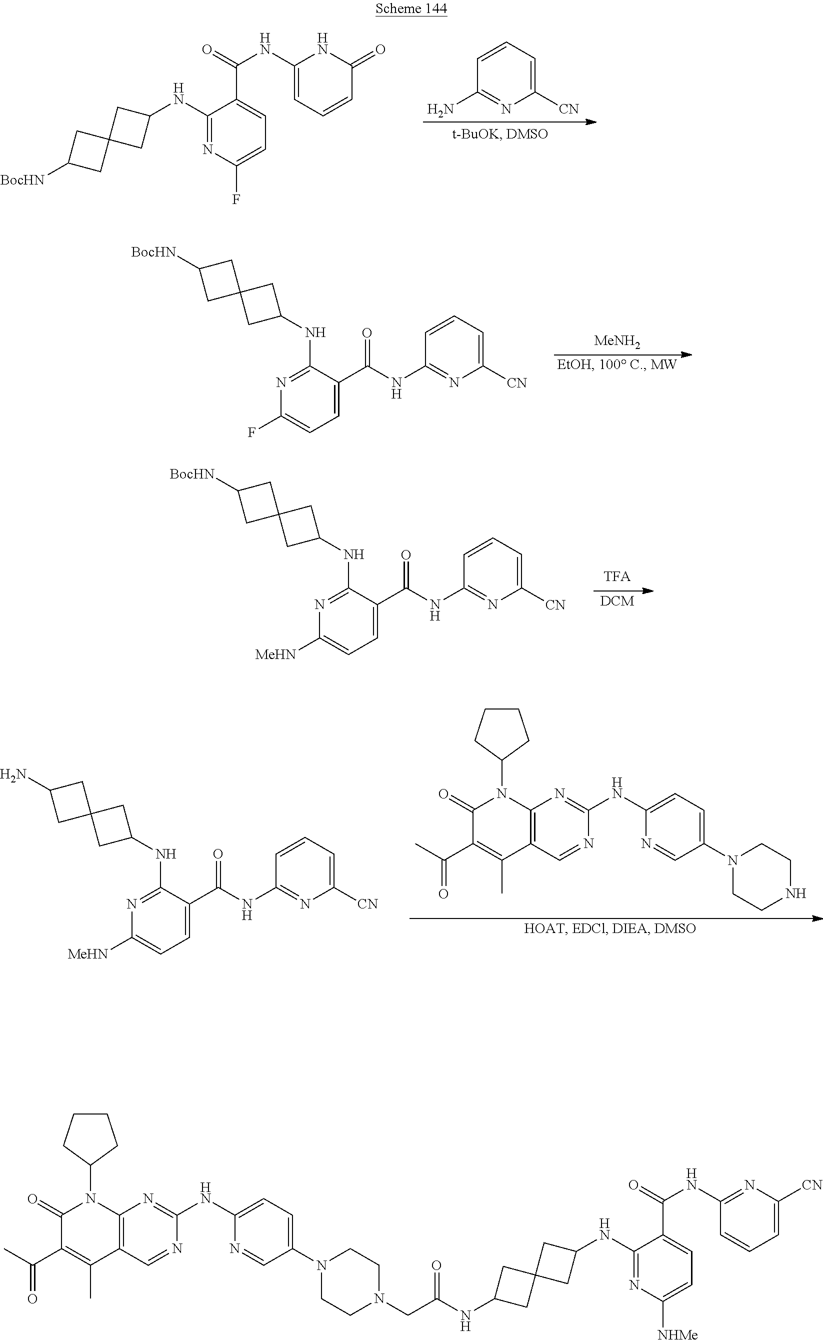

CPD-90 was synthesized following the procedures for preparing CPD-86 and CPD-25 (7.2 mg, 10% yield over 4 steps) as a yellow solid. MS (ESI) m/z=865.7 [M+H]⁺.

### Example 145. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-(trifluoromethyl)pyridin-2-yl)nicotinamide (CPD-91)

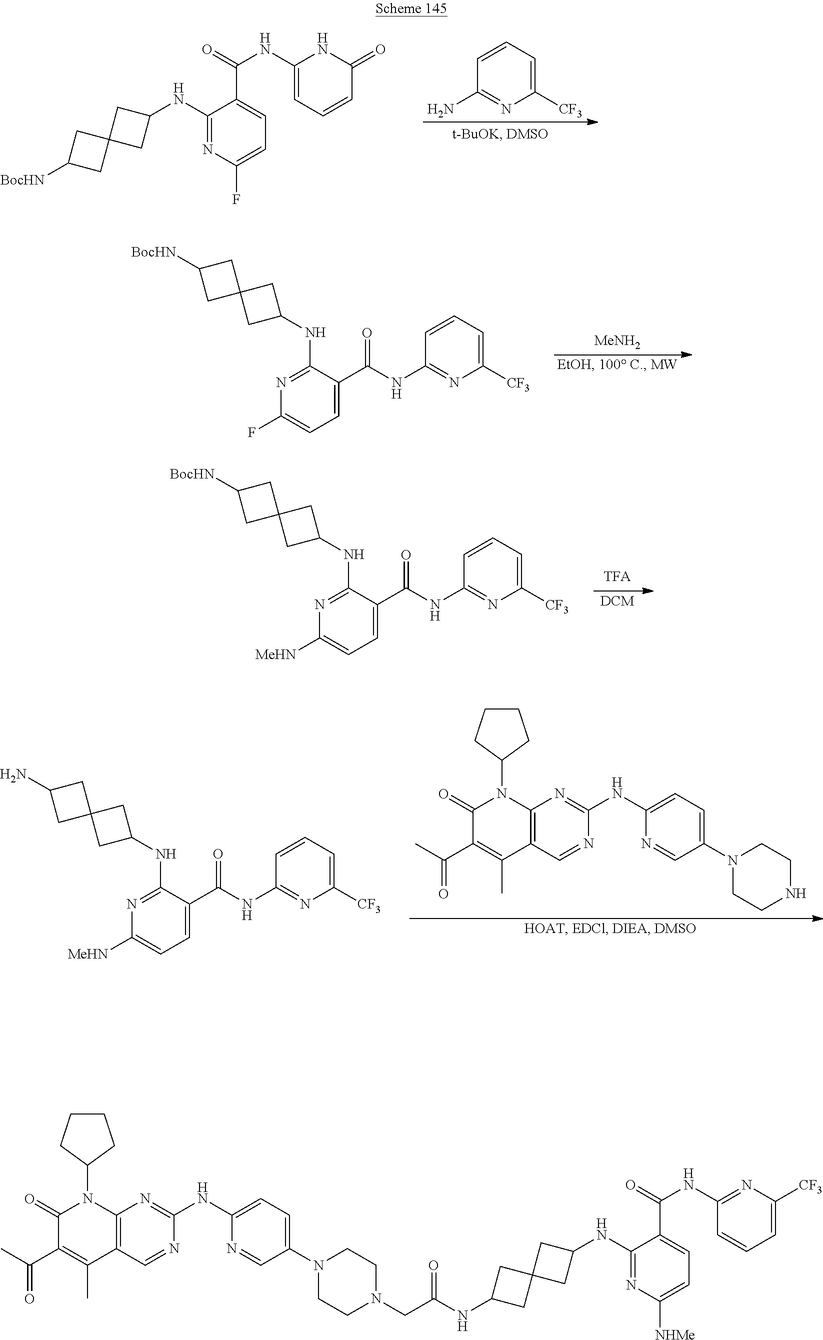

CPD-91 was synthesized following the procedures for preparing CPD-86 and CPD-25 (6.7 mg, 8% yield over 4 steps) as a yellow solid. MS (ESI) m/z=908.7 [M+H]⁺.

### Example 146. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methyl-1H-pyrazol-3-yl)-6-(methylamino)nicotinamide (CPD-92)

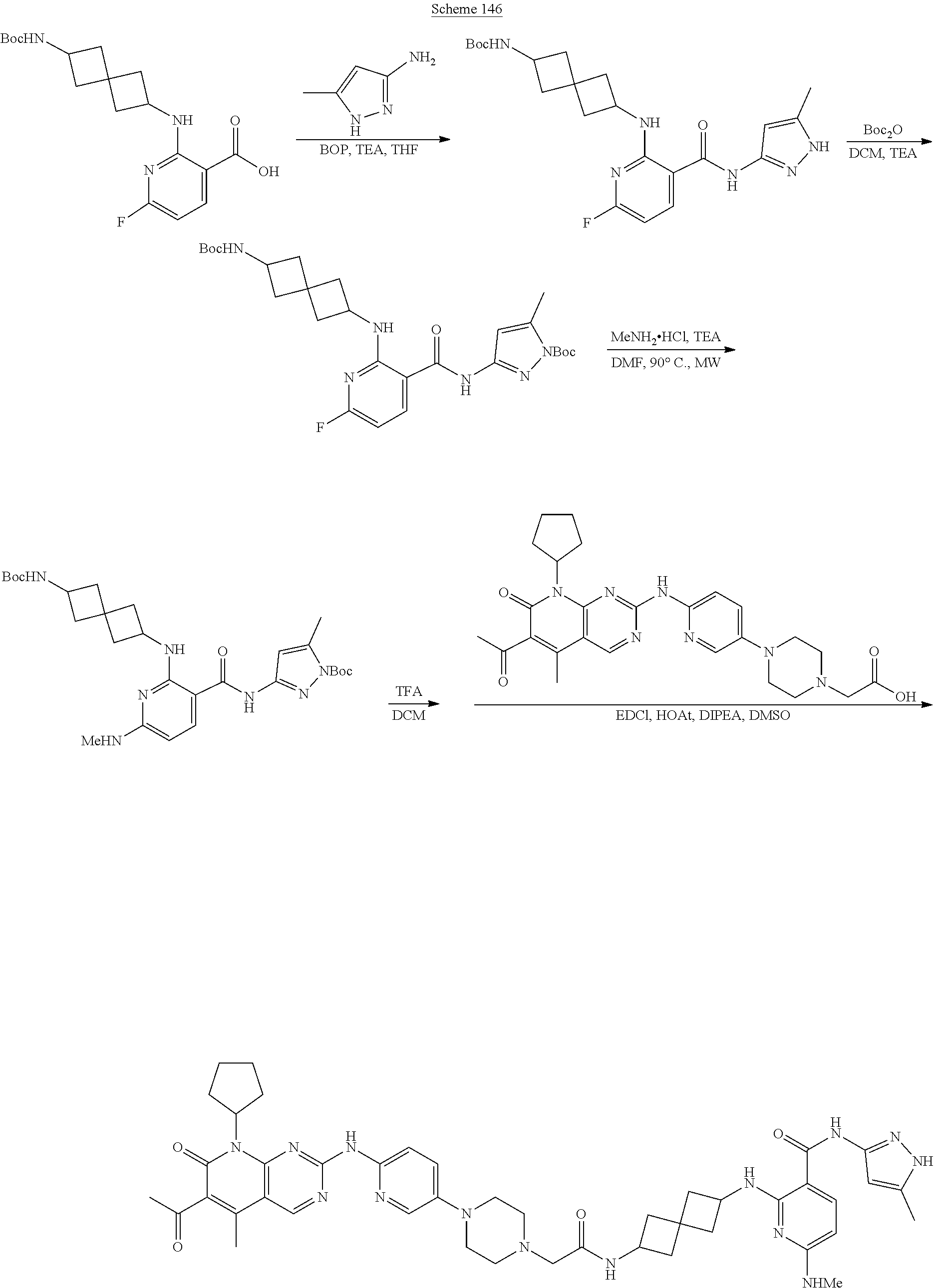

### Step 1. Synthesis of tert-butyl (6-((6-fluoro-3-((5-methyl-1H-pyrazol-3-yl)carbamoyl)pyridin-2-yl)amino)spiro[3.3]heptan-2-yl)carbamate

To a solution of 2-((6-((tert-butoxycarbonyl)amino)spiro[3.3]heptan-2-yl)amino)-6-fluoronicotinic acid (50 mg, 0.14 mmol) in THE (10 mL) were added 5-methyl-1H-pyrazol-3-amine (26 mg, 0.27 mmol), BOP (120 mg, 0.27 mmol) and TEA (140 mg, 1.37 mmol). The reaction mixture was stirred at 40° C. for 2 h. Then the mixture was poured into water (20 mL) and extracted with ethyl acetate (2×30 mL). The combined organic layers were washed with saturated brine (20 mL), dried over anhydrous sodium sulfate, filtered, and evaporated under reduced pressure. The resulting residue was purified by silica gel chromatography to provide the desired product (50 mg, 82% yield) as a light-yellow solid. MS (ESI) m/z=445.7 [M+H]⁺.

### Step 2. Synthesis of tert-butyl 3-(2-((6-((tert-butoxycarbonyl)amino)spiro[3.3]heptan-2-yl)amino)-6-fluoronicotinamido)-5-methyl-1H-pyrazole-1-carboxylate

To a solution of tert-butyl (6-((6-fluoro-3-((5-methyl-1H-pyrazol-3-yl)carbamoyl)pyridin-2-yl)amino)spiro[3.3]heptan-2-yl)carbamate (50 mg, 0.11 mmol) in DCM (6 mL) were added di-tert-butyl dicarbonate (50 mg, 0.22 mmol) and TEA (113 mg, 1.12 mmol). After the reaction mixture was stirred at rt for 4 h, it was poured into water (30 mL) and extracted with ethyl acetate (3×30 mL). The combined organic layers were washed with saturated brine (20 mL), dried over anhydrous sodium sulfate, filtered, and evaporated under reduced pressure. The resulting residue was purified by silica gel chromatography to provide the desired product (40 mg, 65% yield) as a white solid. MS (ESI) m/z=545.5 [M+H]⁺.

### Step 3. Synthesis of tert-butyl 3-(2-((6-((tert-butoxycarbonyl)amino)spiro[3.3]heptan-2-yl)amino)-6-(methylamino)nicotinamido)-5-methyl-1H-pyrazole-1-carboxylate

To a solution of tert-butyl 3-(2-((6-((tert-butoxycarbonyl)amino)spiro[3.3]heptan-2-yl)amino)-6-fluoronicotinamido)-5-methyl-1H-pyrazole-1-carboxylate (40 mg, 73.45 mmol) in DMF (3 mL) were added MeNH₂·HCl (50 mg, 0.73 mmol) and DIEA (95 mg, 0.73 mmol). The reaction mixture was stirred at 90° C. for 2 h. After cooling down to rt, the reaction mixture was quenched with water (20 mL) and extracted with ethyl acetate (3×20 mL). The combined organic layers were washed with saturated brine (20 mL), dried over anhydrous sodium sulfate, filtered, and evaporated under reduced pressure. The resulting residue was purified by silica gel chromatography to provide the desired product (20 mg, 49% yield) as a yellow solid. MS (ESI) m/z=556.8 [M+H]⁺

The remaining steps were performed following the standard procedures for preparing CPD-25 to provide the desired product (4 mg, 13% yield over 2 steps) as a yellow solid. MS (ESI) m/z=843.8 [M+H]⁺.

### Example 147. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-3-yl)-6-(methylamino)nicotinamide (CPD-93)

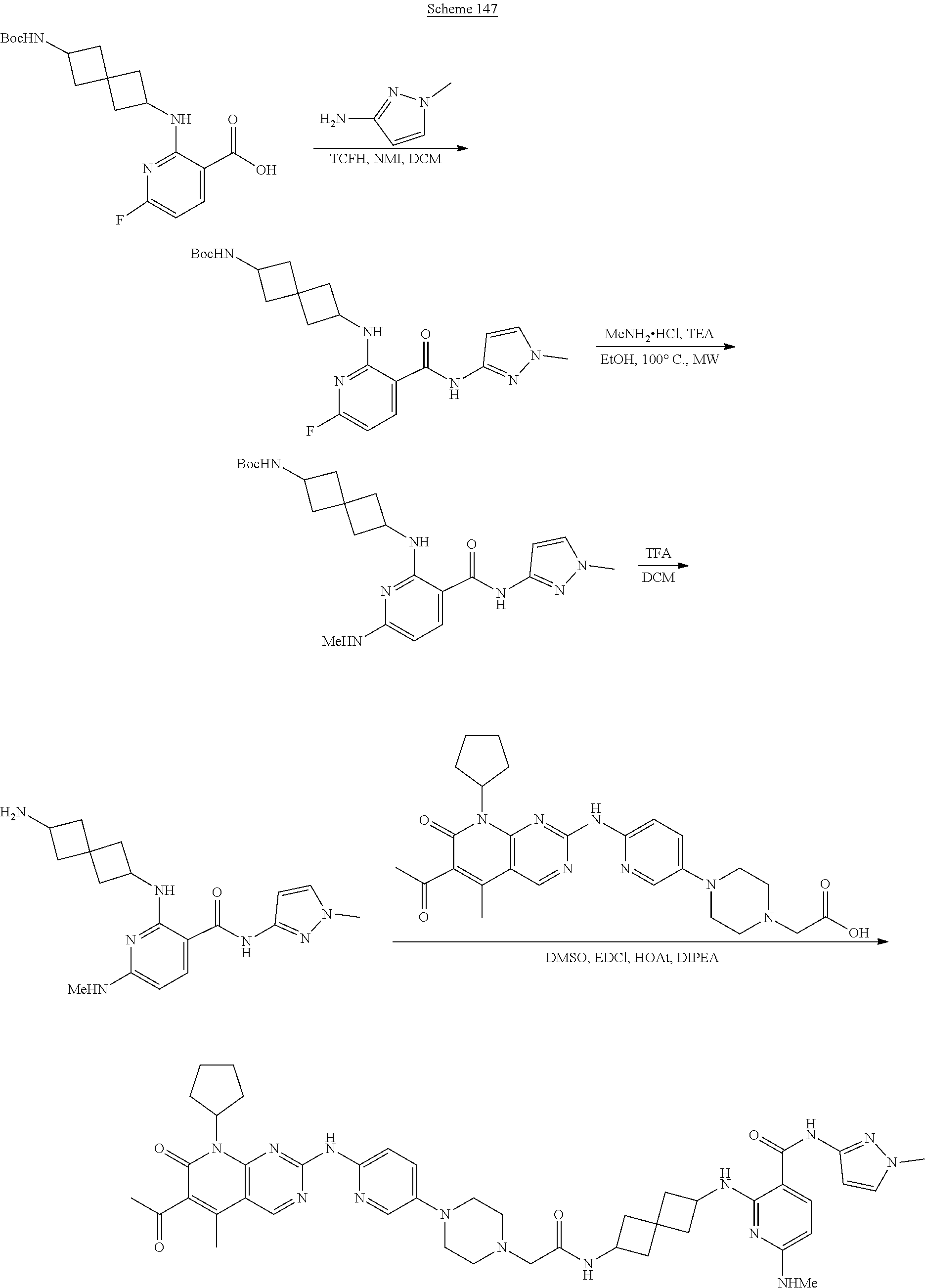

CPD-93 was synthesized following the procedures for preparing CPD-31 (3.7 mg, 8% yield over 4 steps) as a yellow solid. MS (ESI) m/z=843.8 [M+H]⁺.

### Example 148. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-4-yl)-6-(methylamino)nicotinamide (CPD-94)

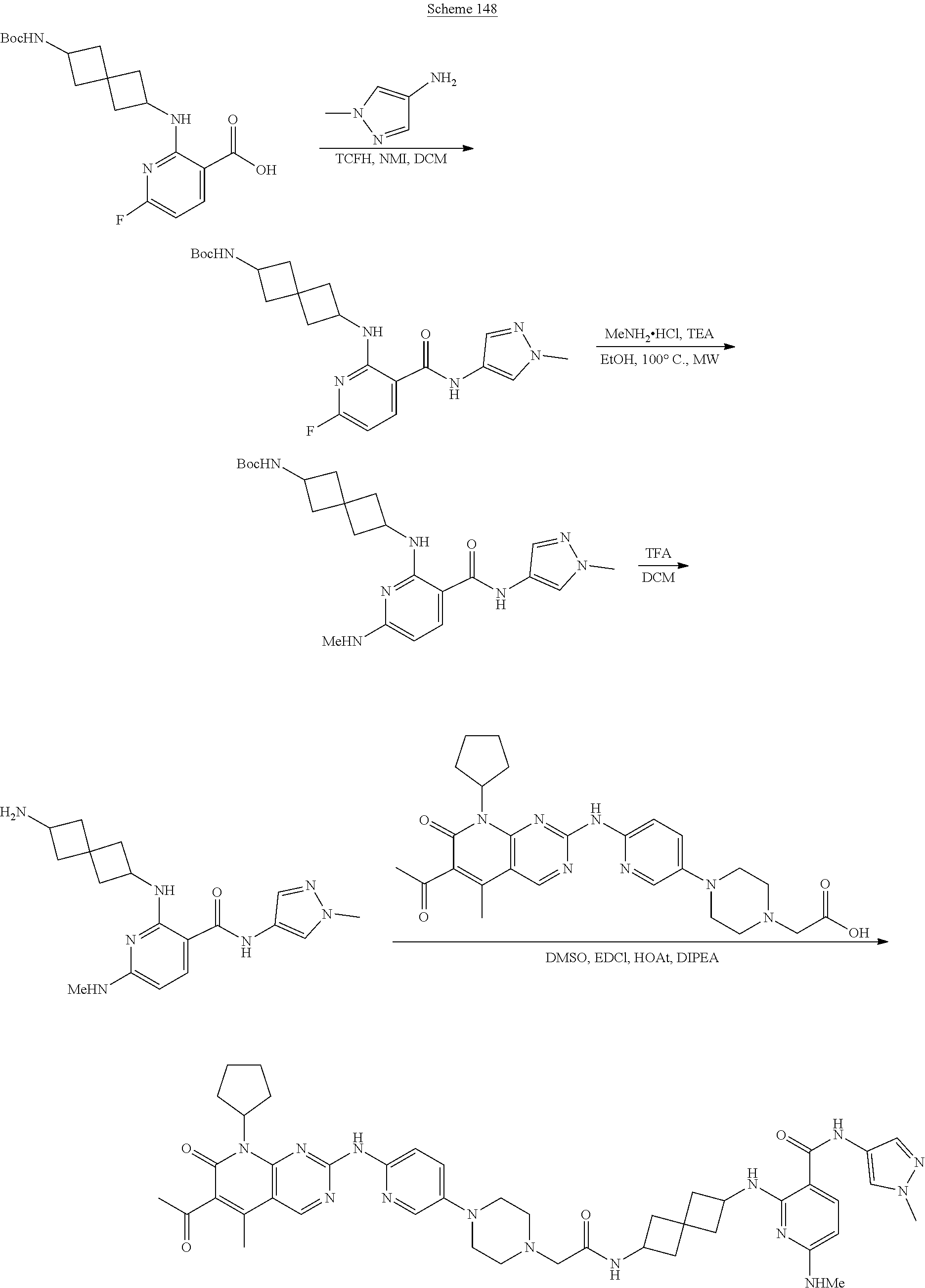

CPD-94 was synthesized following the procedures for preparing CPD-31 (5.5 mg, 12% yield over 4 steps) as a yellow solid. MS (ESI) m/z=843.8 [M+H]⁺.

### Example 149. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-isopropyl-1-methyl-1H-pyrazol-3-yl)-6-(methylamino)nicotinamide (CPD-95)

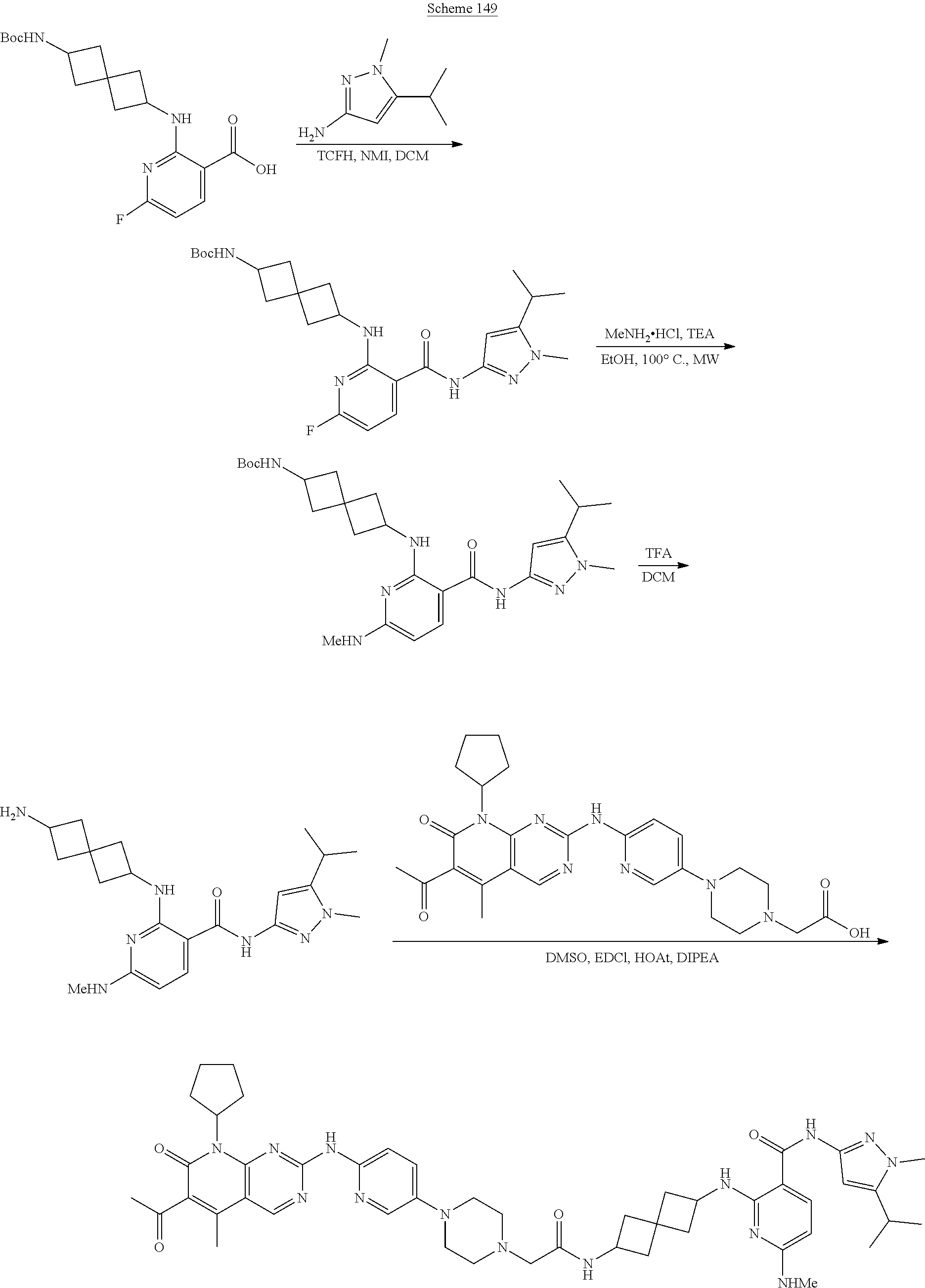

CPD-95 was synthesized following the procedures for preparing CPD-31 (4.8 mg, 10% yield over 4 steps) as a yellow solid. MS (ESI) m/z=885.8 [M+H]⁺.

### Example 150. 2-((6-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)spiro[3.3]heptan-2-yl)amino)-N-(6-(dimethylamino)pyridazin-3-yl)-6-(methylamino)nicotinamide (CPD-96)

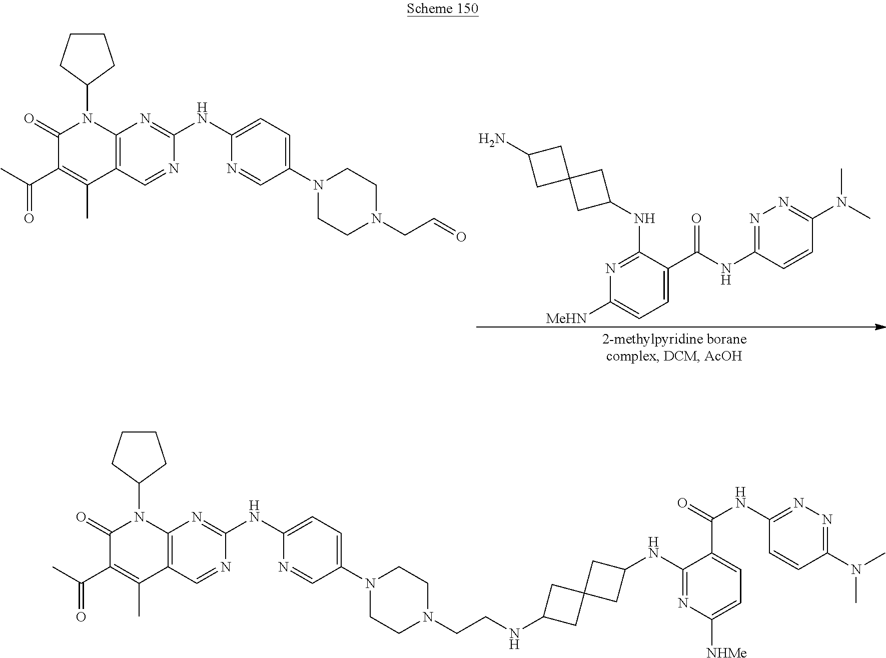 [SMILES: CC(=O)c1c(C)c2cnc(Nc3ccc(N4CCN(CC=O)CC4)cn3)nc2n(C2CCCC2)c1=O ; CNc1ccc(C(=O)Nc2ccc(N(C)C)nn2)c(NC2CC3(CC(N)C3)C2)n1 ; CNc1ccc(C(=O)Nc2ccc(N(C)C)nn2)c(NC2CC3(CC(NCCN4CCN(c5ccc(Nc6ncc7c(C)c(C(C)=O)c(=O)n(C8CCCC8)c7n6)nc5)CC4)C3)C2)n1]

CPD-96 was synthesized following the procedures for preparing CPD-21 (0.8 mg, 2% yield) as a yellow solid. MS (ESI) m/z=870.9 [M+H]⁺.

### Example 151. 2-((2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetyl)-2-azaspiro[3.31heptan-6-yl)amino)-N-(6-(dimethylamino)pyridazin-3-yl)-6-(methylamino)nicotinamide (CPD-97)

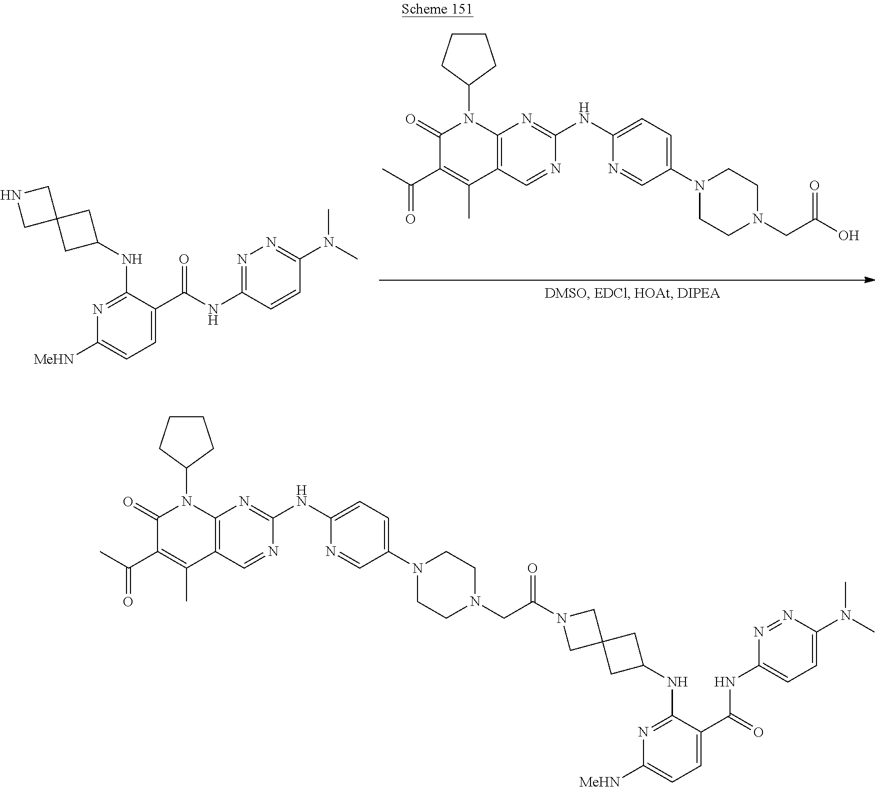 [SMILES: CNc1ccc(C(=O)Nc2ccc(N(C)C)nn2)c(NC2CC3(CNC3)C2)n1 ; CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(NC2CC3(C2)CN(C(=O)CN2CCN(c4ccc(Nc5ncc6c(C)c(C(C)=O)c(=O)n(C7CCCC7)c6n5)nc4)CC2)C3)n1]

CPD-97 was synthesized following the procedures for preparing CPD-1 (5.2 mg, 44% yield) as a yellow solid. MS (ESI) m/z=870.8 [M+H]⁺.

### Example 152. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyclopropyl-1-methyl-1H-pyrazol-3-yl)-6-(methylamino)nicotinamide (CPD-98)

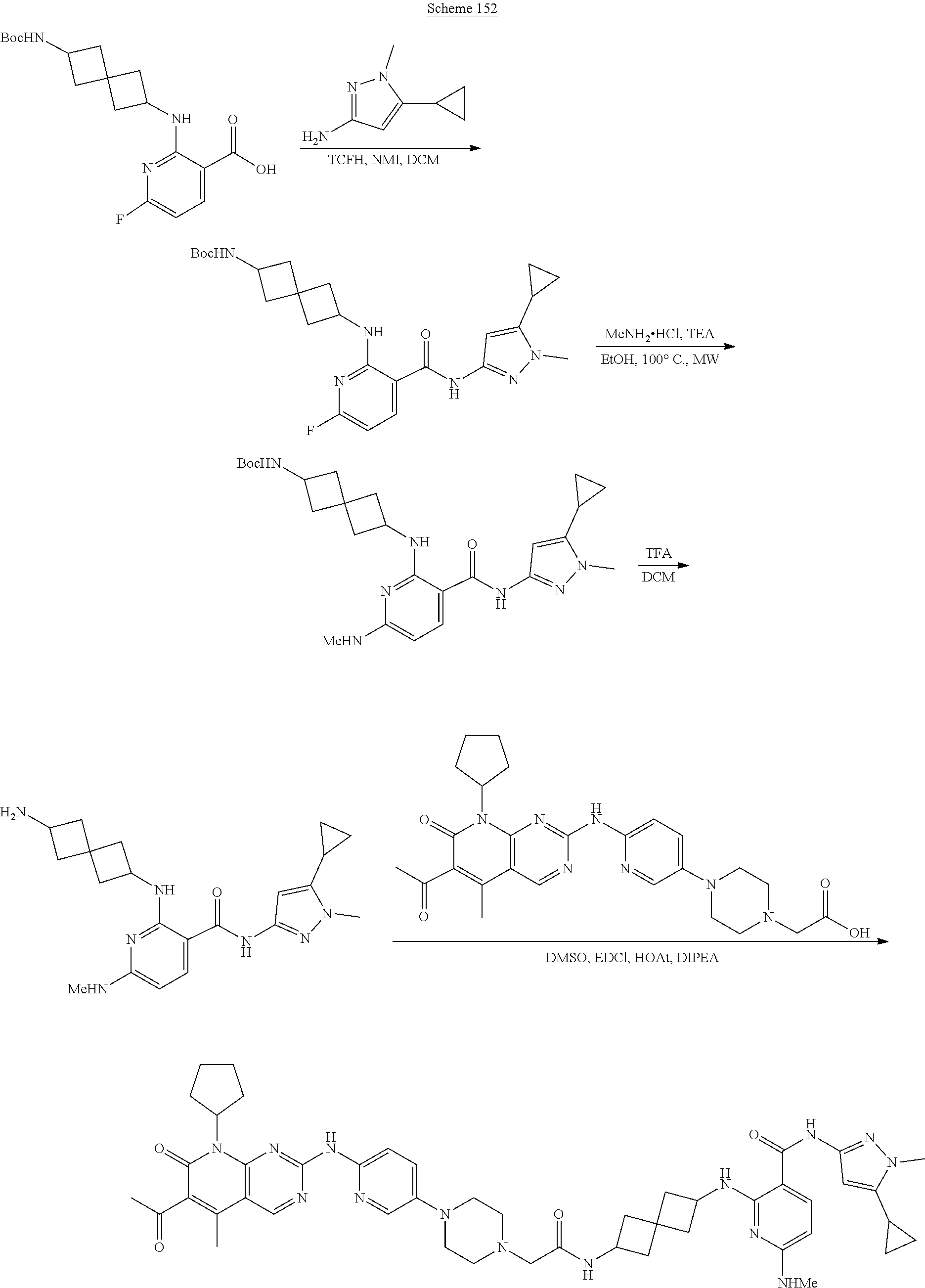

CPD-98 was synthesized following the procedures for preparing CPD-31 (2.0 mg, 2% yield over 4 steps) as a yellow solid. MS (ESI) m/z=883.8 [M+H]⁺.

### Example 153. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-isopropyl-5-methyl-1H-pyrazol-3-yl)-6-(methylamino)nicotinamide (CPD-99)

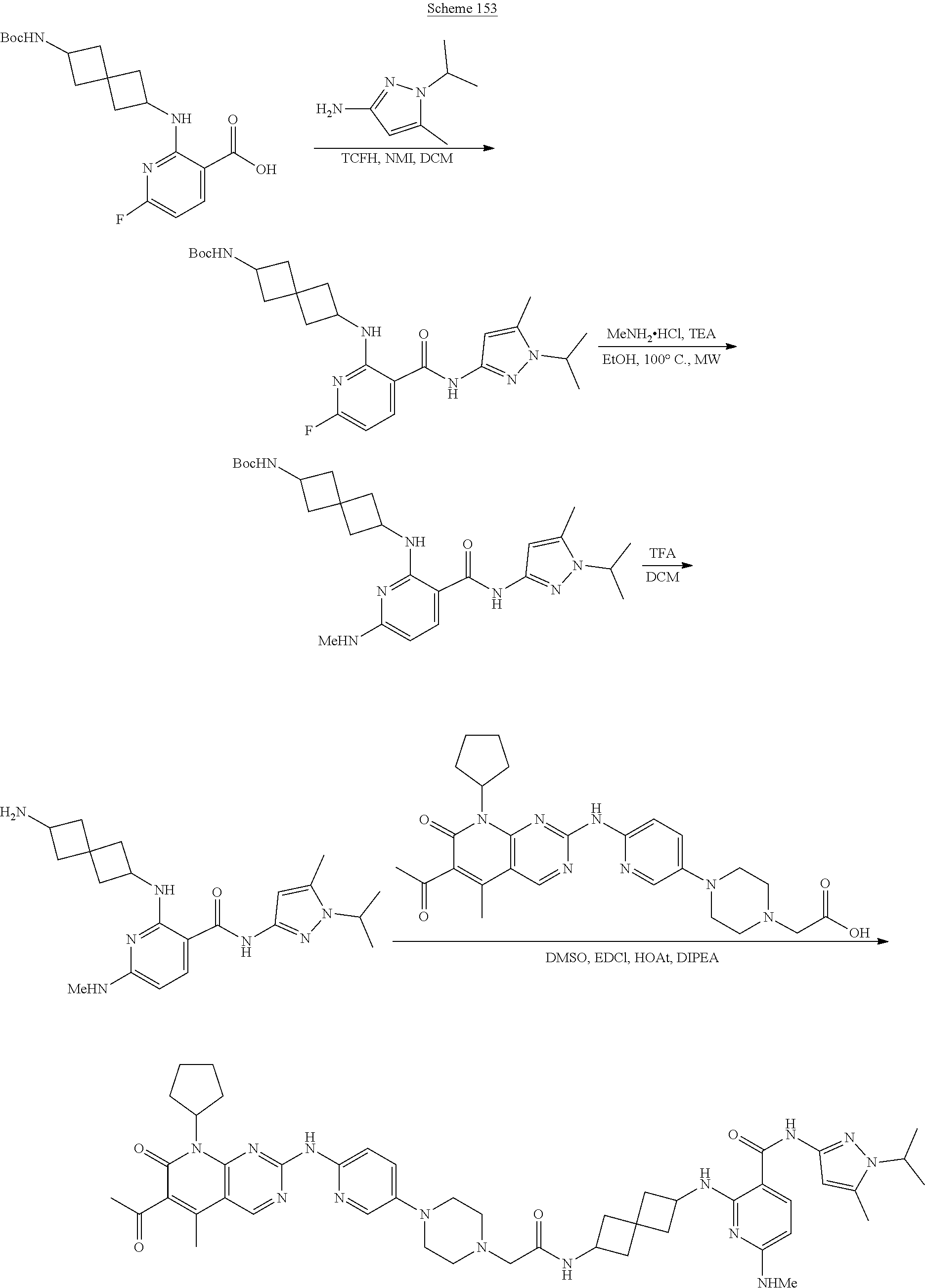

CPD-99 was synthesized following the procedures for preparing CPD-31 (5.0 mg, 4% yield over 4 steps) as a yellow solid. MS (ESI) m/z=885.9 [M+H]⁺.

### Example 154. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(1H-pyrazol-3-yl)nicotinamide (CPD-100)

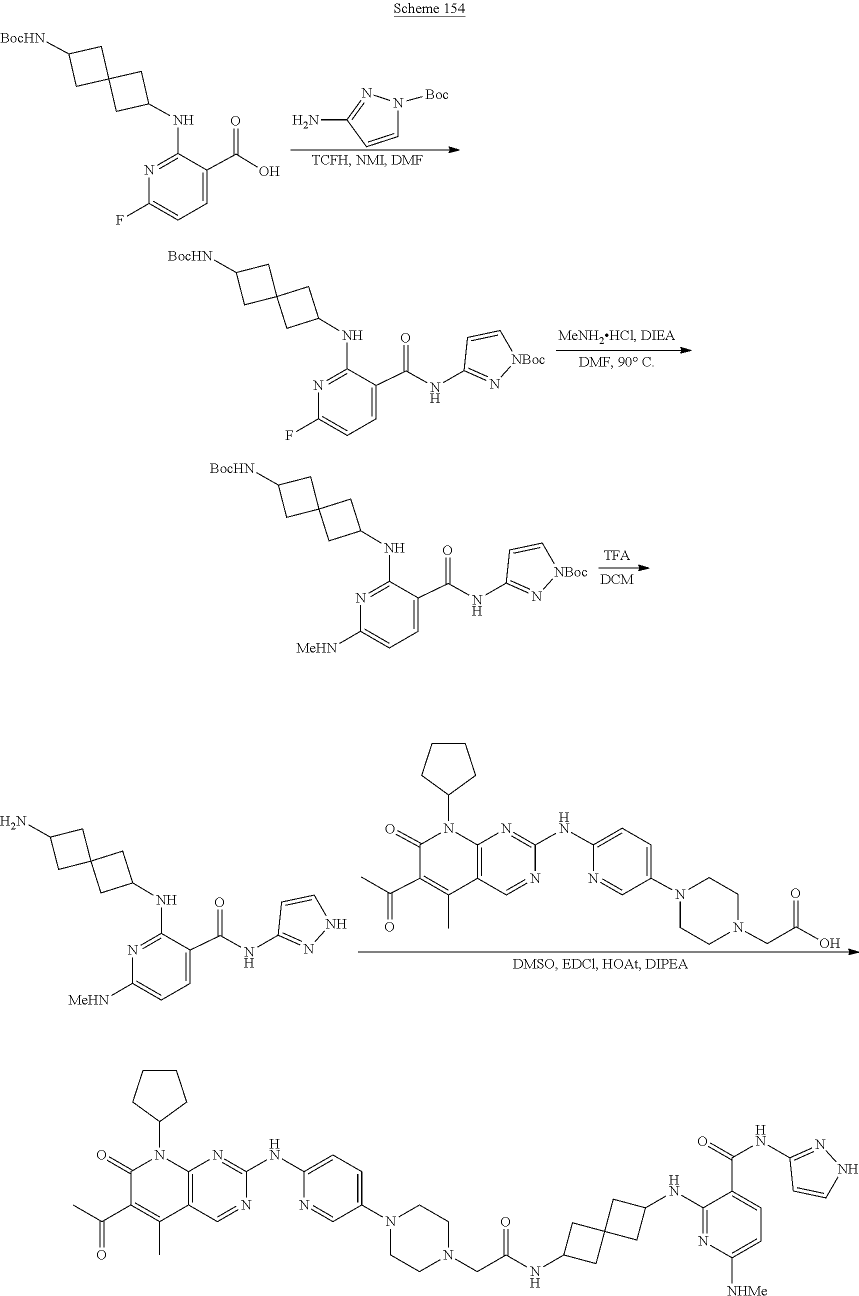

CPD-100 was synthesized following the procedures for preparing CPD-31 (5.0 mg, 7% yield over 4 steps) as a yellow solid. MS (ESI) m/z=829.7 [M+H]⁺.

### Example 155. 2-((3-(2-((2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)ethyl)amino)-2-oxoethyl)cyclobutyl)amino)-N-(6-(dimethylamino)pyridazin-3-yl)-6-(methylamino)nicotinamide (CPD-101)

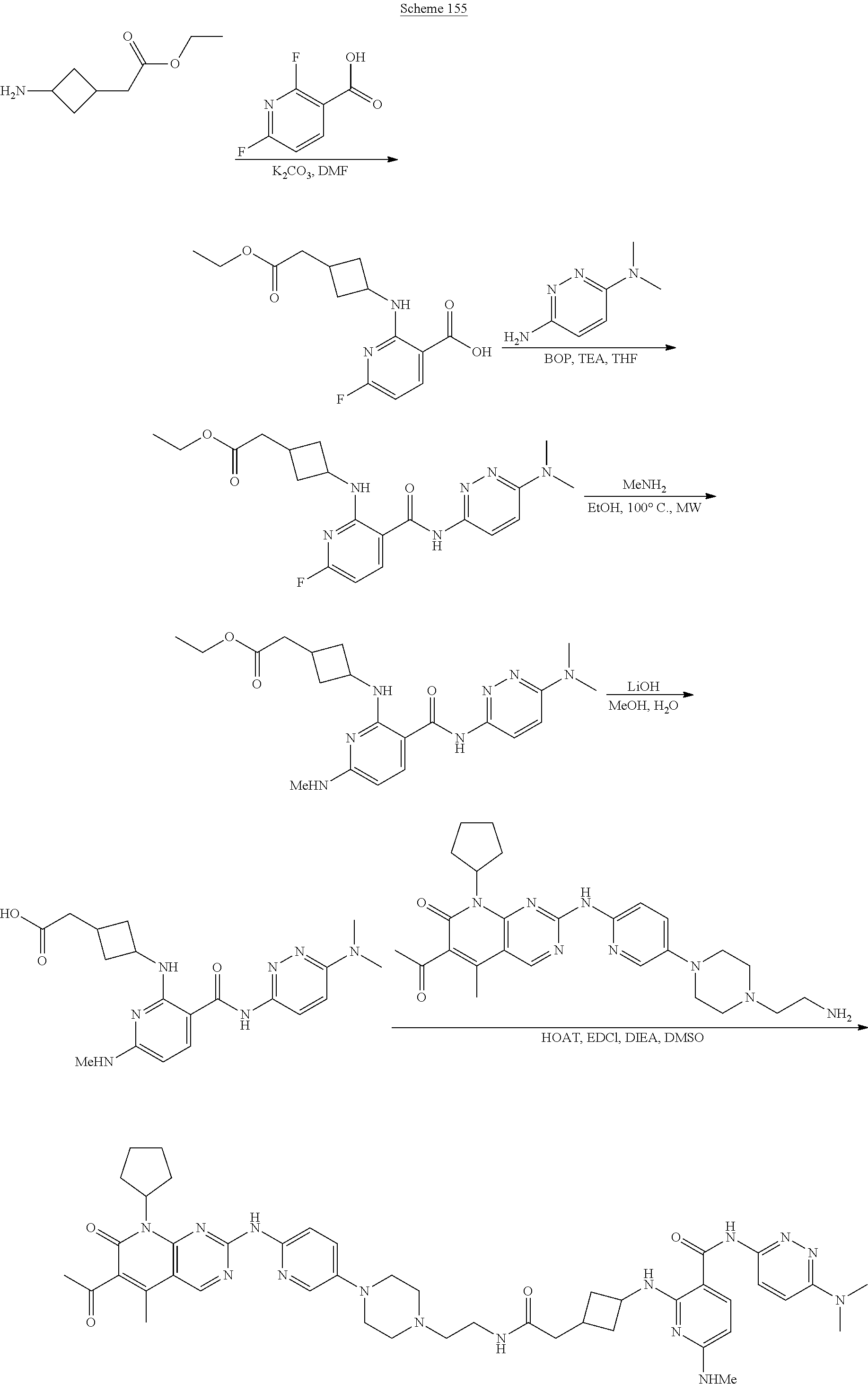

CPD-101 was synthesized following the procedures for preparing CPD-82 (2.0 mg, 1% yield over 5 steps) as a yellow solid. MS (ESI) m/z=872.9 [M+H]⁺.

### Example 156. 2-((3-(1-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetyl)azetidin-3-yl)propyl)amino)-N-(6-(dimethylamino)pyridazin-3-yl)-6-(methylamino)nicotinamide (CPD-102)

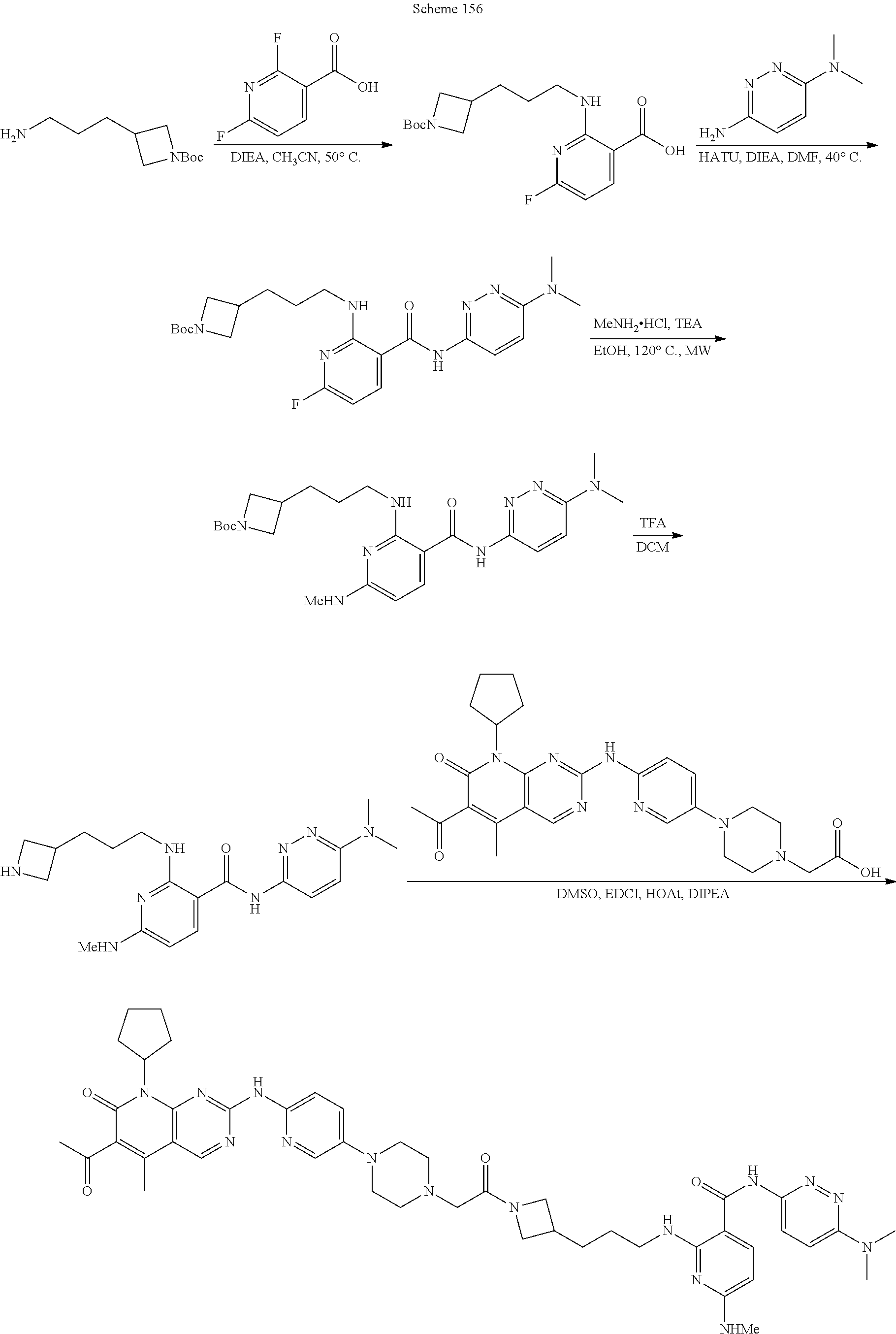

CPD-102 was synthesized following the procedures for preparing CPD-34 and CPD-32 (2.9 mg, 2% yield over 5 steps) as a yellow solid. MS (ESI) m/z=872.9 [M+H]⁺.

### Example 157. 2-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-cyclopropylpyridin-2-yl)-6-(methylamino)nicotinamide (CPD-103)

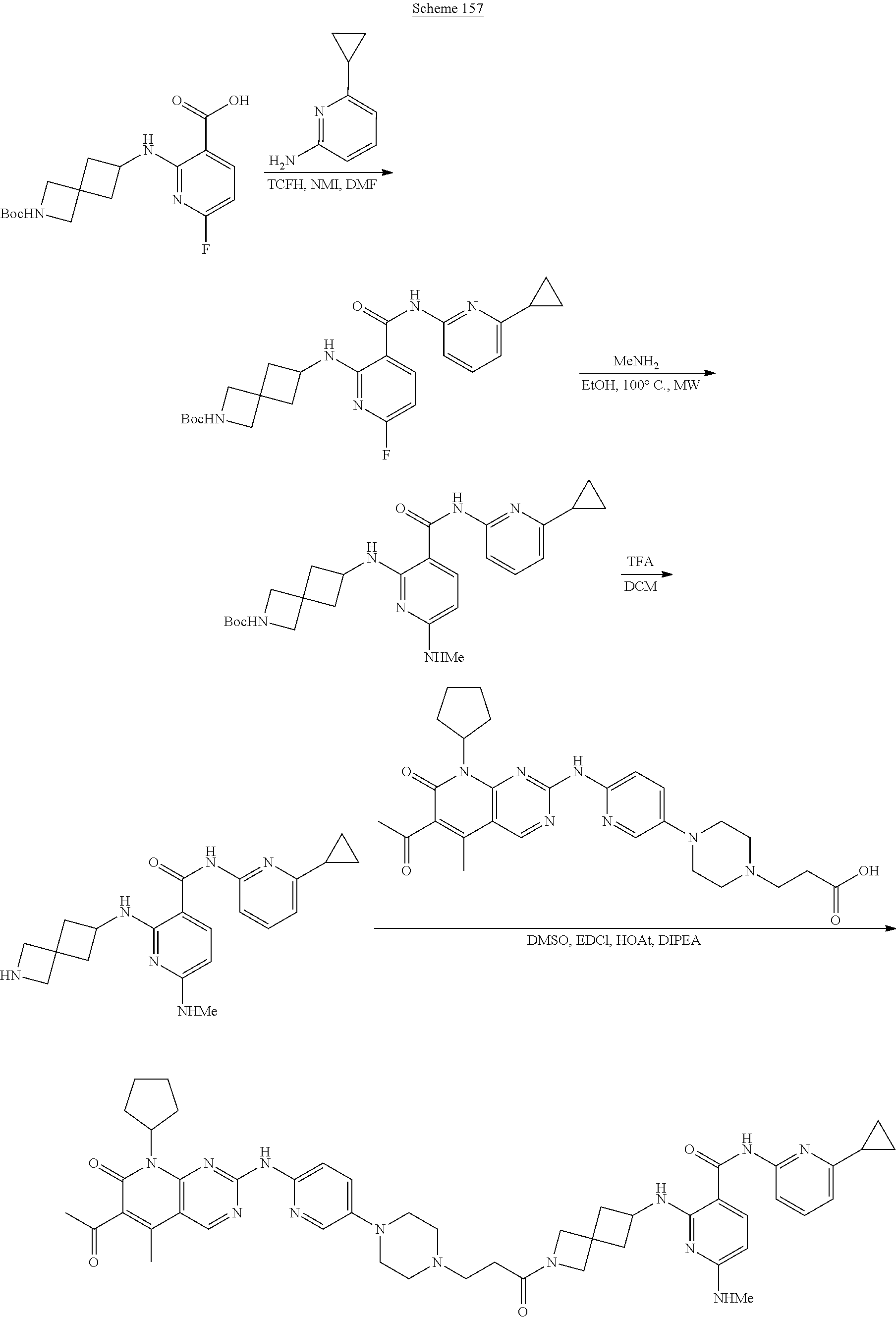

CPD-103 was synthesized following the procedures for preparing CPD-31 (8.9 mg, 30% yield over 4 steps) as a yellow solid. MS (ESI) m/z=880.8 [M+H]⁺.

### Example 1582. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-aminopyridazin-3-yl)-6-(methylamino)nicotinamide (CPD-104)

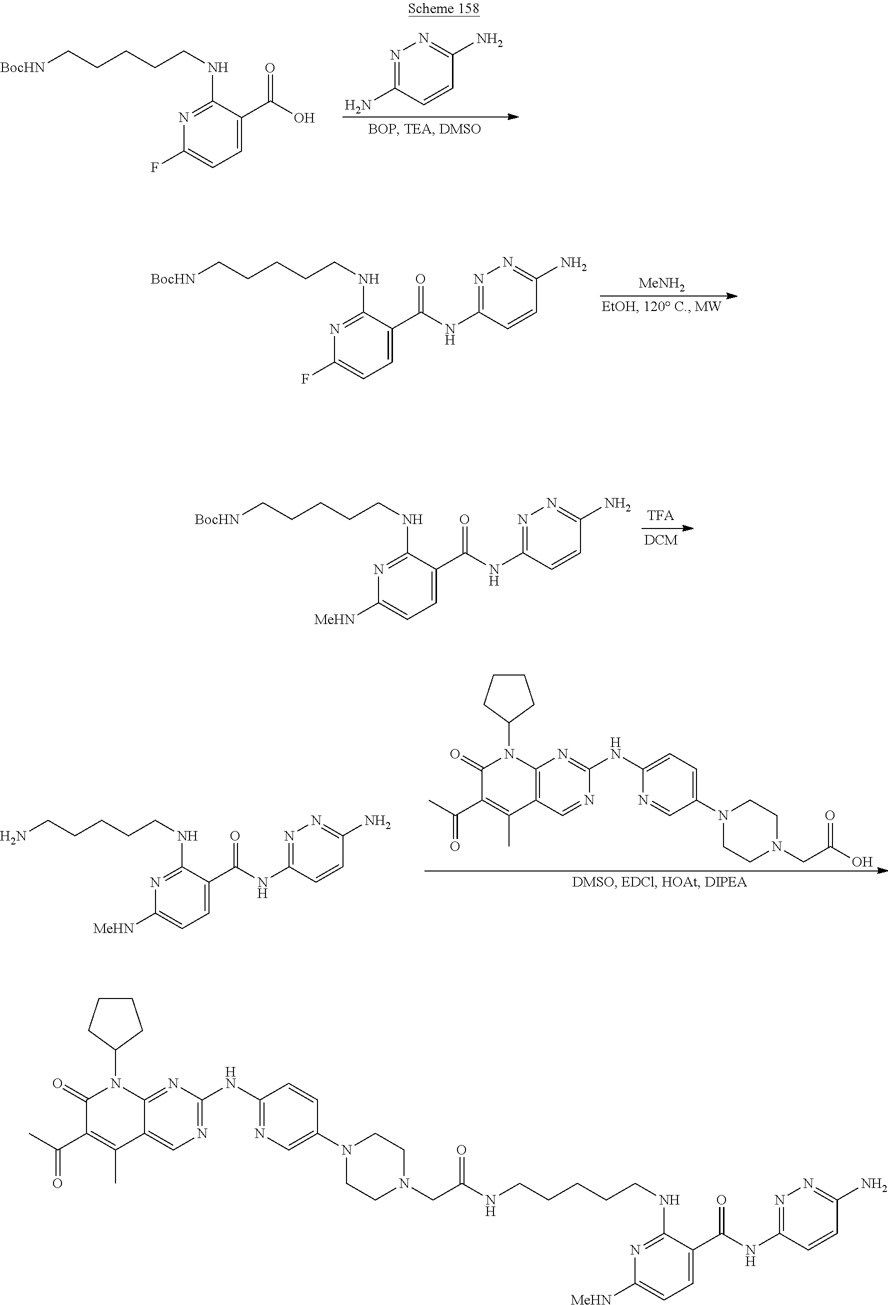

CPD-104 was synthesized following the procedures for preparing CPD-25 (9.5 mg, 5% yield over 4 steps) as a yellow solid. MS (ESI) m/z=417.1 [M/2+H]⁺.

### Example 159. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-methylpyridazin-3-yl)nicotinamide (CPD-105)

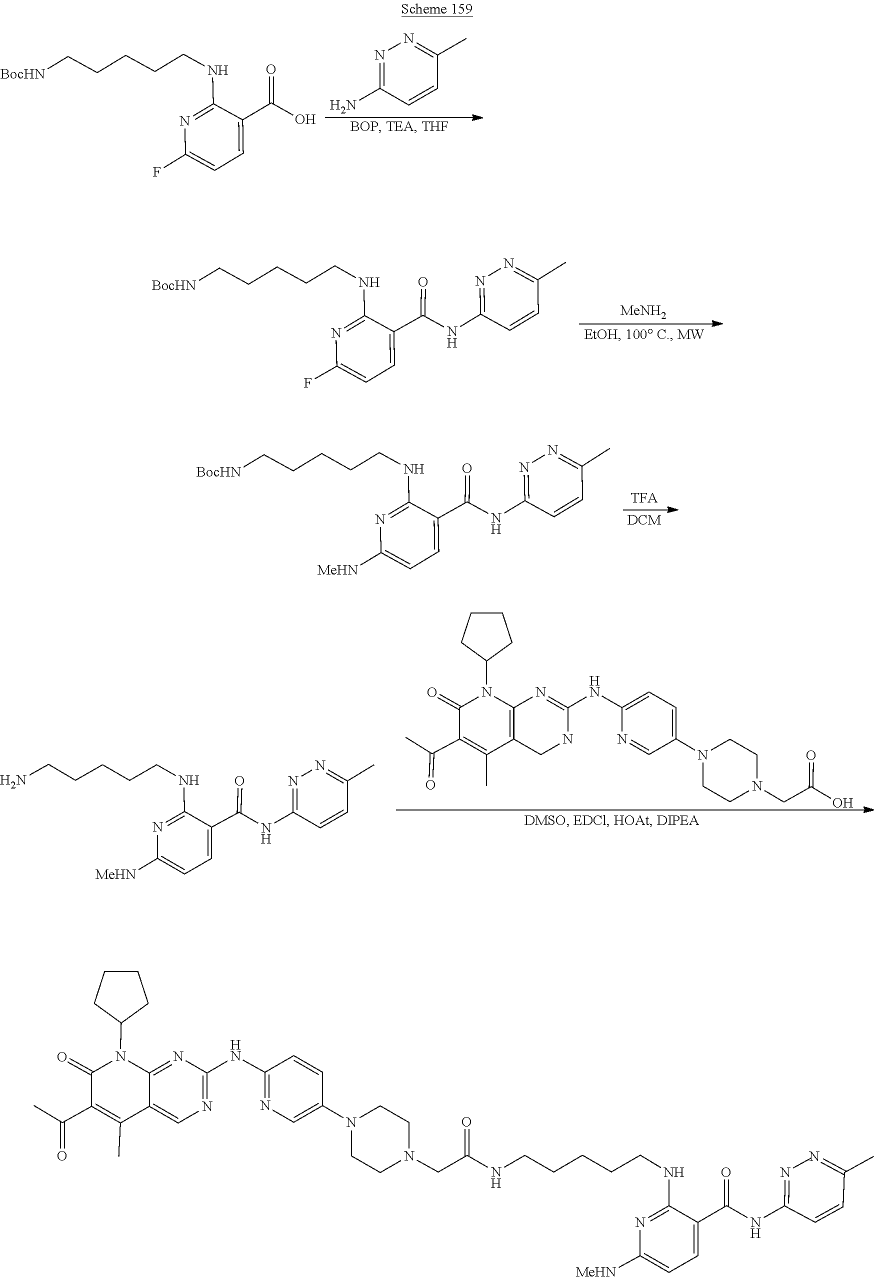

CPD-105 was synthesized following the procedures for preparing CPD-25 (4.4 mg, 3% yield over 4 steps) as a yellow solid. MS (ESI) m/z=416.6 [M/2+H]⁺.

### Example 160. 2-((1-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)pyrrolidin-3-yl)amino)-N-(6-(dimethylamino)pyridazin-3-yl)-6-(methylamino)nicotinamide (CPD-106)

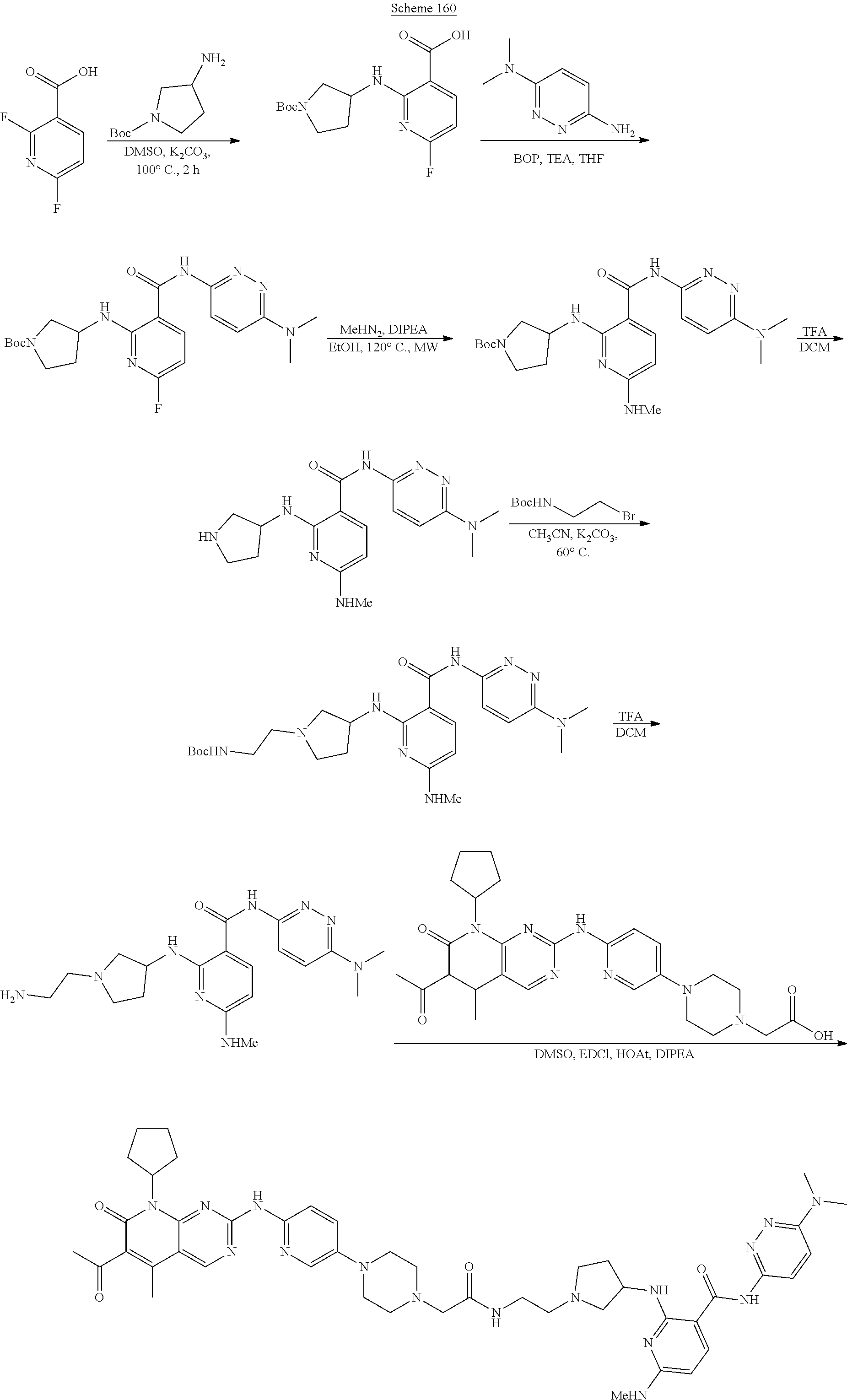

### Step 1. Synthesis of 2-((1-(tert-butoxycarbonyl)pyrrolidin-3-yl)amino)-6-fluoronicotinic acid

To a mixture of 2,6-difluoronicotinic acid (500 mg, 3.14 mmol) in DMSO (5 mL) were added tert-butyl 3-methylpyrrolidine-1-carboxylate (643.9 mg, 3.46 mmol), potassium carbonate (1.30 g, 9.43 mmol). The mixture was stirred at 80° C. for 2 h. After cooling down to rt, the mixture was diluted with H₂O and adjusted pH to 3 by aq. HCl solution (1N). The solution was extracted with DCM. The organic layer was washed with brine, dried over sodium sulfate, filtered, and concentrated. The residue was purified by silica gel chromatography (DCM/MeOH=25:1) to provide the title product (510 mg, 50% yield) as a pink solid. MS (ESI) m/z=384.4 [M+H]⁺.

### Step 2. Synthesis of tert-butyl 3-((3-((6-(dimethylamino)pyridazine-3-yl)carbamoyl)-6-fluoropyridin-2-yl)amino)pyrrolidine-1-carboxylate

To a mixture of 2-((1-(tert-butoxycarbonyl)pyrrolidin-3-yl)amino)-6-fluoronicotinic acid (200 mg, 614.75 μmol) in THF (6 mL) were added M-N³-dimethylpyridazine-3,6-diamine (84.94 mg, 614.75 μmol), 1H-benzotriazol-1-yloxytris(dimethylamino)phosphonium hexafluorophosphate (407.84 mg, 922.13 μmol) and TEA. The reaction mixture was stirred at 50° C. for 14 h. After cooling down to rt, the mixture was diluted with H₂O and extracted with DCM. The organic layer was washed with brine, dried over sodium sulfate, filtered, and concentrated. The residue was purified by silica gel chromatography (DCM/MeOH=20:1) to provide the title product (220 mg, crude) as a yellow gum. MS (ESI) m/z=446.5 [M+H]⁺.

### Step 3. Synthesis of tert-butyl 3-((3-((6-(dimethylamino)pyridazin-3-yl)carbamoyl)-6-(methyl amino)pyridin-2-yl)amino)pyrrolidine-1-carboxylate

To a mixture of tert-butyl 3-((3-((6-(dimethylamino)pyridazin-3-yl)carbamoyl)-6-fluoropyridin-2-yl)amino)pyrrolidine-1-carboxylate (40 mg, 89.79 μmol) in EtOH (1.5 mL) were added methylamine hydrochloride (30.31 mg, 448.94 μmol) and DIPEA (58.02 mg, 448.94 mol). The mixture was stirred at 120 cc for 2 h under microwave irradiation. After cooling down to rt, the mixture was diluted with H₂O and extracted with DCM. The organic layer was washed with brine, dried over sodium sulfate, filtered, and concentrated. The residue was purified by prep-TLC (DCM/MeOH=15:1) to provide the title product (14 mg, 34% yield) as a yellow gum. MS (ESI) m/z=457.6 [M+H]⁺.

### Step 4. Synthesis of N-(6-(dimethylamino)pyridazin-3-yl)-6-(methylamino)-2-(pyrrolidin-3-yl-amino)nicotinamide 2,2,2-trifluoroacetic acid

To a mixture of tert-butyl 3-((3-((6-(dimethylamino)pyridazin-3-yl)carbamoyl)-6-(methylamino)-pyridin-2-yl)amino)pyrrolidine-1-carboxylate (30 mg, 65.71 μmol) in DCM (3 mL) was added TFA (1.5 mL). The mixture was stirred at rt for 2 h. The mixture was concentrated to provide crude product (30 mg, crude) as a yellow gum. MS (ESI) m/z=357.5 [M+H]⁺.

### Step 5. Synthesis of tert-butyl (2-(3-((3-((6-(dimethylamino)pyridazin-3-yl)carbamoyl)-6-(methylamino)pyridin-2-yl)amino)pyrrolidin-1-yl)ethyl)carbamate

To a mixture of N-(6-(dimethylamino)pyridazin-3-yl)-6-(methylamino)-2-(pyrrolidin-3-ylamino)nicotinamide 2,2,2-trifluoroacetic acid (30 mg, 63.77 μmol) in CH₃CN (4 mL) were added tert-butyl (2-bromoethyl)carbamate (21.44 mg, 95.65 μmol) and potassium carbonate (26.44 mg, 191.31 μmol). The mixture was stirred at 60° C. for 5 h. After cooling down to rt, the mixture was diluted with H₂O and extracted with DCM. The organic layer was washed with brine, dried over sodium sulfate, filtered, and concentrated. The residue was purified by silica gel chromatography (DCM/MeOH=15:1) to provide the title product (18 mg, 57% yield) as a yellow solid. MS (ESI) m/z=500.6 [M+H]⁺.

The remaining steps were performed according to the standard procedures to provide the desired product (5.95 mg, 19% yield) as a yellow solid. MS (ESI) m/z=887.8 [M+H]⁺.

### Example 161. 2-((1-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)azetidin-3-yl)amino)-N-(6-(dimethylamino)pyridazin-3-yl)-6-(methylamino)nicotinamide (CPD-107)

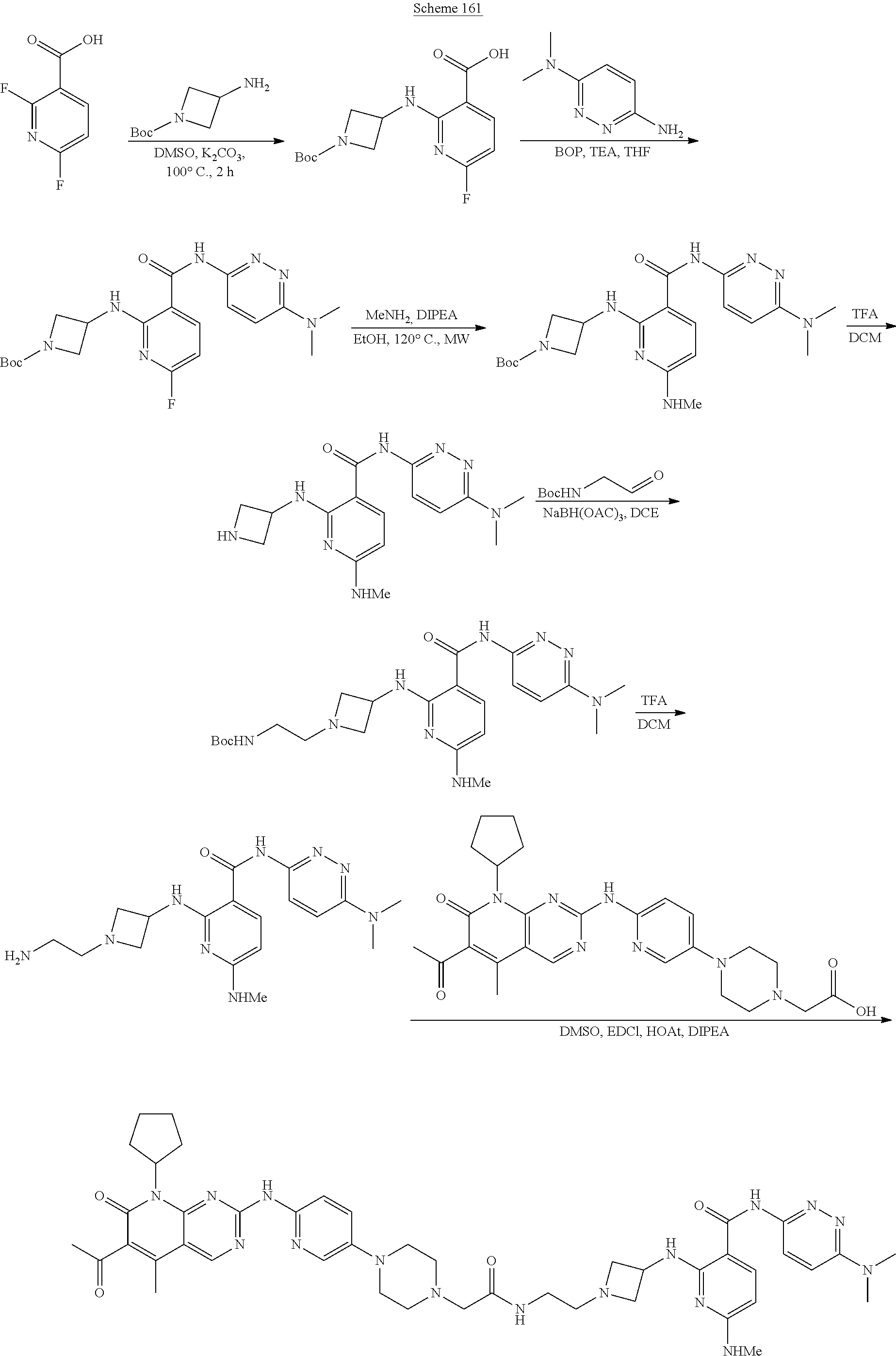

### Step 1 to 4. Synthesis of 2-(azetidin-3-ylamino)-N-(6-(dimethylamino)pyridazin-3-yl)-6-(methylamino) nicotinamide 2,2,2-trifluoroacetic acid

The title compound was synthesized following the procedures of step 1 to 4 for preparing CPD-106 (170 mg, 14% yield over 4 steps) as a yellow gum. MS (ESI) m/z=343.5 [M+H]⁺.

### Step 5. Synthesis of tert-butyl (2-(3-((3-((6-(dimethylamino)pyridazin-3-yl)carbamoyl)-6-(methyl amino)pyridin-2-yl)amino)azetidin-1-yl)ethyl)carbamate

To a mixture of 2-(azetidin-3-ylamino)-N-(6-(dimethylamino)pyridazin-3-yl)-6-(methylamino) nicotinamide 2,2,2-trifluoroacetic acid (170 mg, 372.46 μmol) in DCE (6 mL) were added tert-butyl N-(2-oxoethyl)carbamate (118.58 mg, 744.93 μmol) and sodium triacetoxyborohydride (157.88 mg, 744.93 μmol). The mixture was stirred at rt for 4 h. The mixture was diluted with H₂O and extracted with DCM. The organic layer was washed with brine, dried over sodium sulfate, filtered, and concentrated. The residue was purified by silica gel chromatography (DCM/MeOH=15:1) to provide the title product (15 mg, 8% yield) as a yellow gum. MS (ESI) m/z=486.6 [M+H]⁺.

The remaining steps were performed according to the standard procedures to provide the desired product (3.5 mg, 15% yield over 2 steps) as a yellow solid. MS (ESI) m/z=873.9 [M+H]⁺.

### Example 162. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-methoxypyridazin-3-yl)-6-(methylamino)nicotinamide (CPD-108)

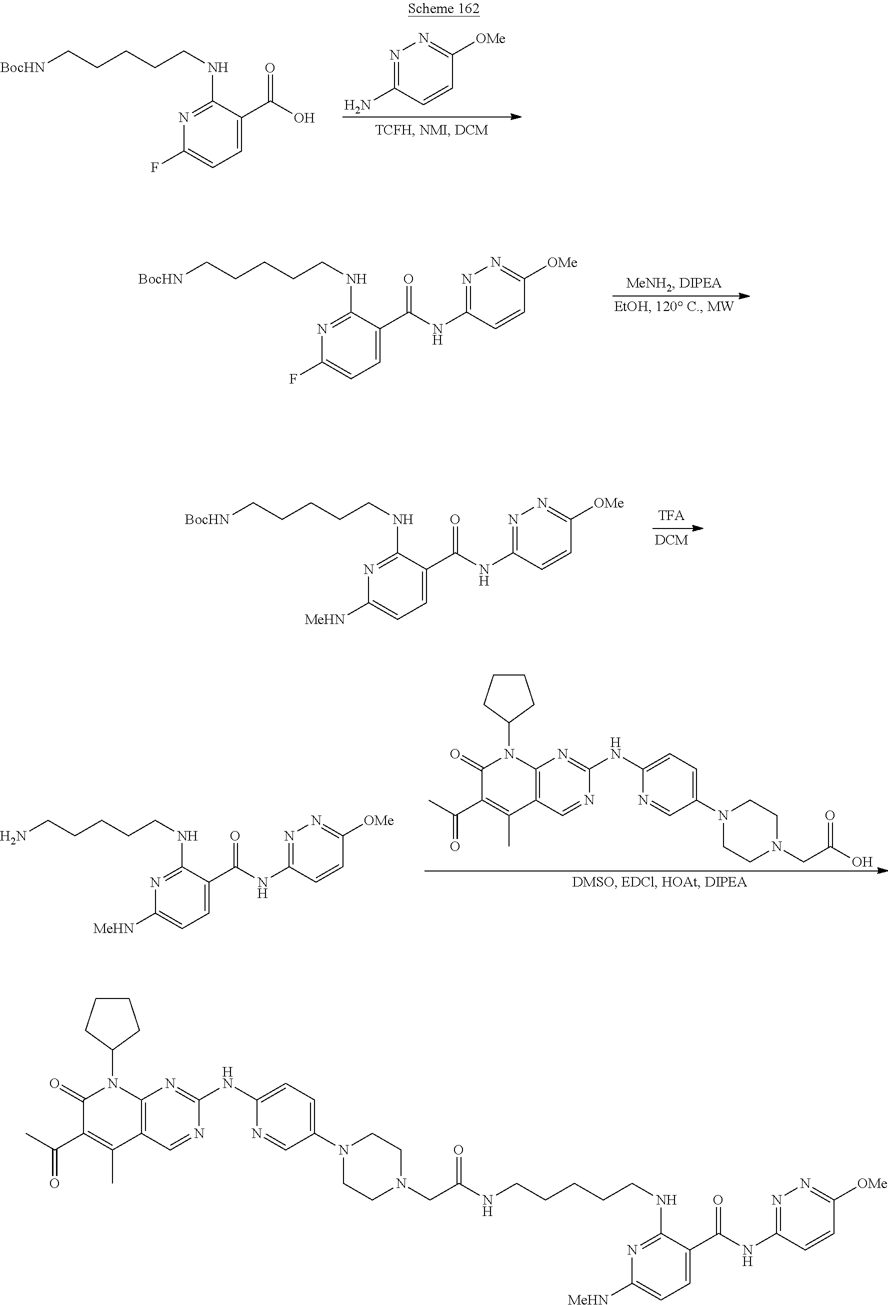

CPD-108 was synthesized following the procedures for preparing CPD-31 (18 mg, 15% yield over 4 steps) as a yellow solid. MS (ESI) m/z=847.8 [M+H]⁺.

### Example 163. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5,6-dimethylpyridin-2-yl)-6-(methylamino)nicotinamide (CPD-109)

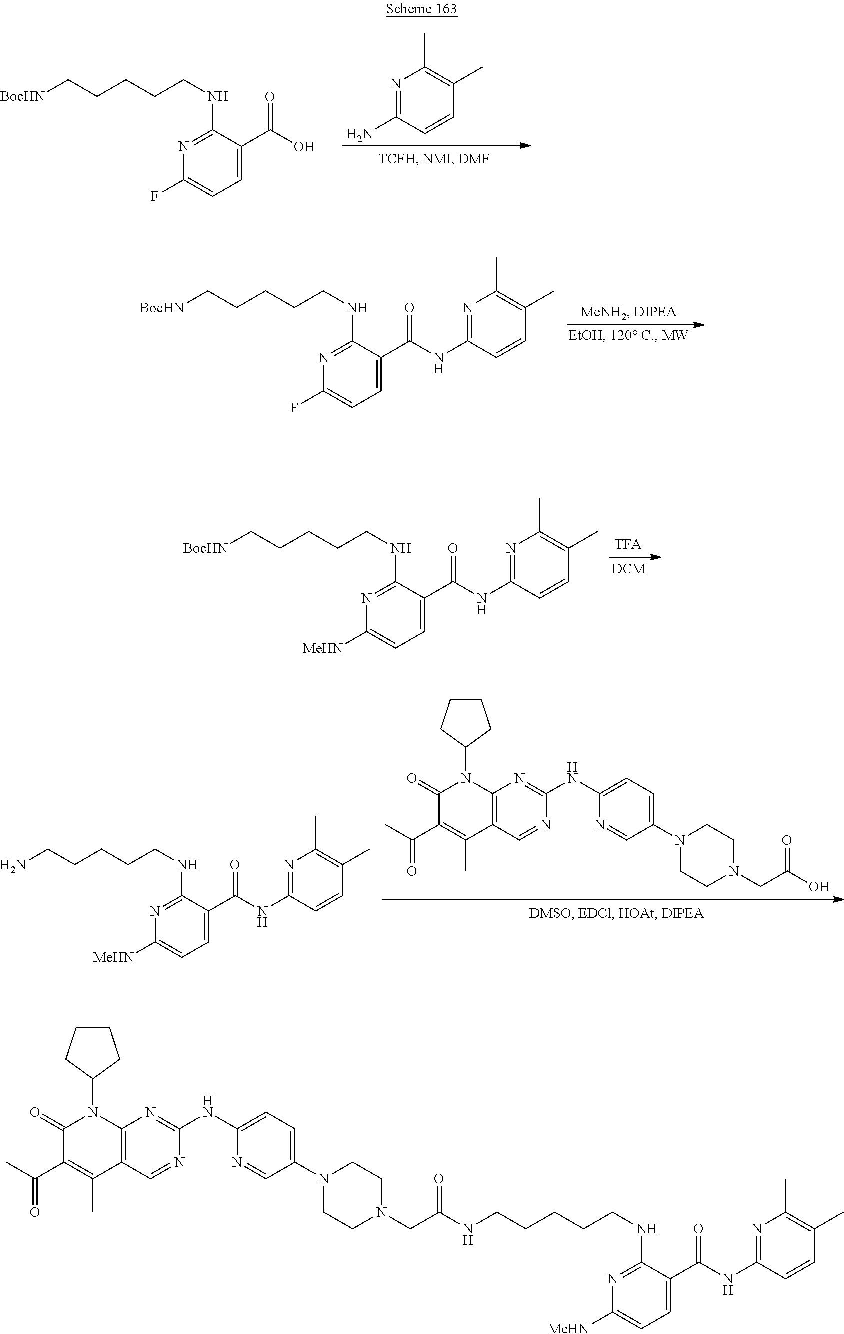

CPD-109 was synthesized following the procedures for preparing CPD-31 (14.4 mg, 1% yield over 4 steps) as a yellow solid. MS (ESI) m/z=844.8 [M+H]⁺.

### Example 164. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-6-(methylamino)-N-(6-(methylamino)pyridazin-3-yl)nicotinamide (CPD-110)

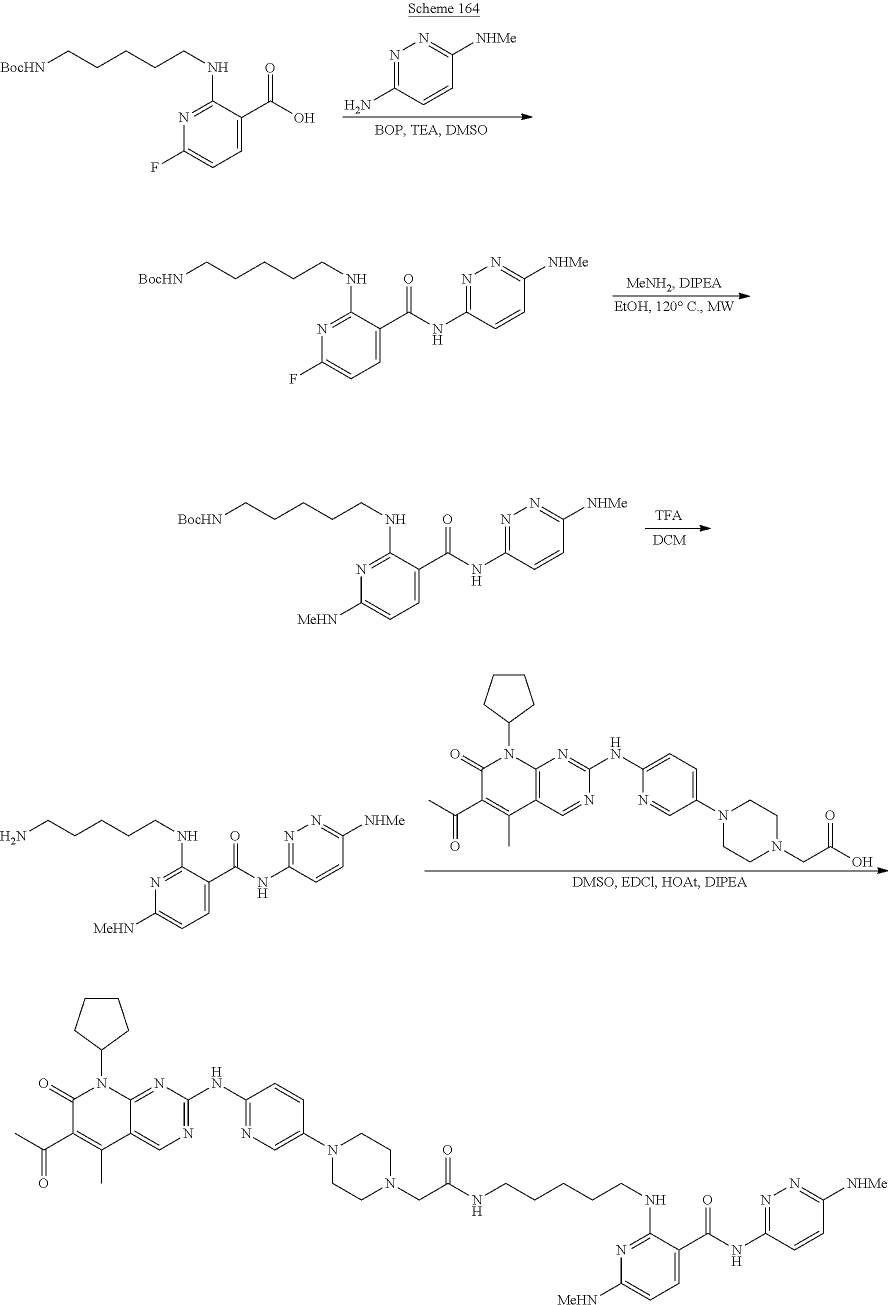

CPD-110 was synthesized following the procedures for preparing CPD-25 (4.4 mg, 1% yield over 4 steps) as a yellow solid. MS (ESI) m/z=846.7 [M+H]⁺.

### Example 165. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-isopropyl-5-methylpyridin-2-yl)-6-(methylamino)nicotinamide (CPD-1H)

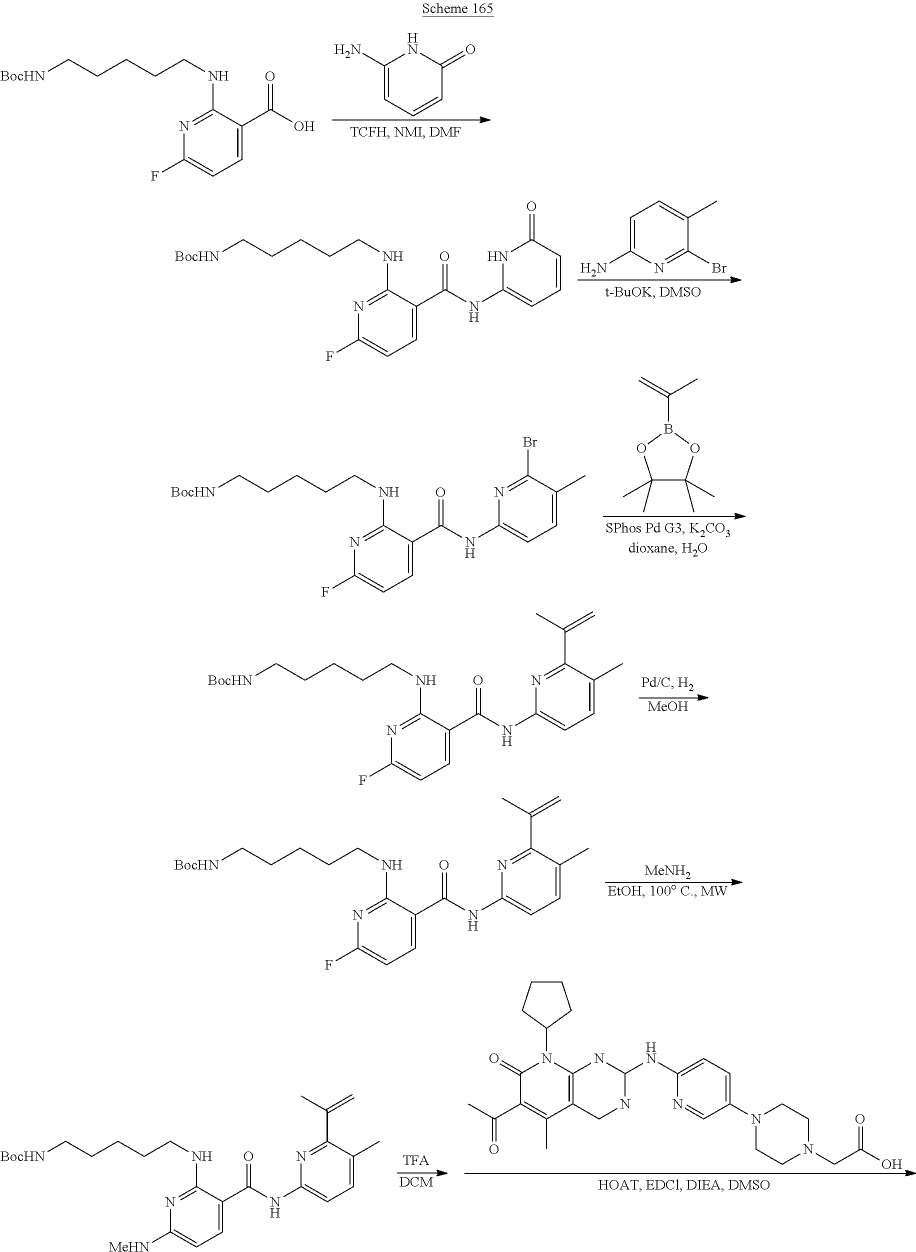

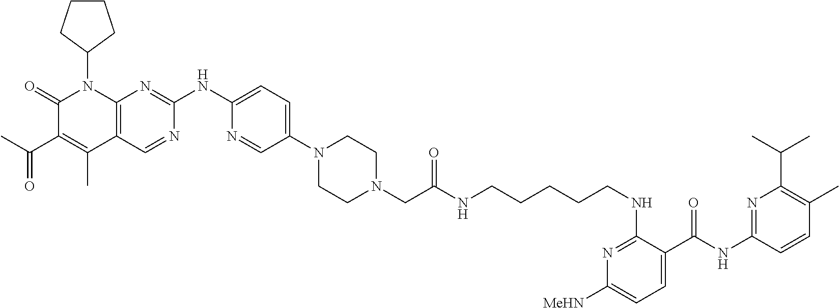 [SMILES: CNc1ccc(C(=O)Nc2ccc(C)c(C(C)C)n2)c(NCCCCCNC(=O)CN2CCN(c3ccc(Nc4ncc5c(C)c(C(C)=O)c(=O)n(C6CCCC6)c5n4)nc3)CC2)n1]

### Step 1. Synthesis of tert-butyl (5-((6-fluoro-3-((6-oxo-1,6-dihydropyridin-2-yl)carbamoyl)pyridin-2-yl)amino)pentyl)carbamate

To a solution of 2-((5-((tert-butoxycarbonyl)amino)pentyl)amino)-6-fluoronicotinic acid (300.0 mg, 0.88 mmol) in DMF (5.0 mL) were added 6-aminopyridin-2(1H)-one (96.8 mg, 0.88 mmol), TCFH (369.1 mg, 1.32 mmol) and NMI (252.5 mg, 3.08 mmol). The reaction mixture was stirred at rt for 2 h. The reaction mixture was poured into water (50.0 mL) and extracted with ethyl acetate (3×20.0 mL). The combined organic layers were washed with saturated brine (30.0 mL), dried over anhydrous sodium sulfate, filtered, and evaporated under reduced pressure. The resulting residue was purified by reverse-phase chromatography to provide the desired product (280.0 mg, 74% yield) as a pale white solid. MS (ESI) m/z=434.6 [M+H]⁺.

### Step 2. Synthesis of tert-butyl (5-((3-((6-bromo-5-methylpyridin-2-yl)carbamoyl)-6-fluoropyridin-2-yl)amino)pentyl)carbamate

To a solution of tert-butyl (5-((6-fluoro-3-((6-oxo-1,6-dihydropyridin-2-yl)carbamoyl)pyridin-2-yl)amino)pentyl)carbamate (100.0 mg, 0.23 mmol) in DMSO (2 mL) were added 6-bromo-5-methylpyridin-2-amine (51.78 mg, 276.83 mmol) and t-BuOK (1M in THF, 0.9 mL). After the reaction mixture was stirred at rt for 0.5 h, it was poured into water (50 mL) and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography to provide the desired product (50.0 mg, 42% yield) as a pale white solid. MS (ESI) m/z=510.4 [M+H]⁺.

### Step 3. Synthesis of tert-butyl (5-((6-fluoro-3-((5-methyl-6-(prop-1-en-2-yl)pyridin-2-yl)carbamoyl)pyridin-2-yl)amino)pentyl)carbamate

To a solution of tert-butyl (5-((3-((6-bromo-5-methylpyridin-2-yl)carbamoyl)-6-fluoropyridin-2-yl)amino)pentyl)carbamate (50.0 mg, 0.098 mmol) in dioxane (5 mL) and water (1 mL) were added 4,4,5,5-tetramethyl-2-(prop-1-en-2-yl)-1,3,2-dioxaborolane (16.46 mg, 0.098 mmol), SPhos Pd G3 (8.49 mg, 9.80 μmol) and K₂CO₃ (13.52 mg, 0.098 mmol) at rt. The reaction mixture was stirred at 100° C. under inert atmosphere for 16 h. After cooling down to rt, the reaction mixture was quenched with water (30 mL) and extracted with ethyl acetate (3×20 mL). The combined organic layers were washed with saturated brine (30 mL), dried over anhydrous sodium sulfate, filtered, and evaporated under reduced pressure. The resulting residue was purified by silica gel chromatography to provide the desired product (30.0 mg, 65% yield) as a pale white solid. MS (ESI) m/z=472.9 [M+H]⁺.

### Step 4. Synthesis of tert-butyl (5-((6-fluoro-3-((6-isopropyl-5-methylpyridin-2-yl)carbamoyl)pyridin-2-yl)amino)pentyl)carbamate

To a solution of tert-butyl (5-((6-fluoro-3-((5-methyl-6-(prop-1-en-2-yl)pyridin-2-yl)carbamoyl)pyridin-2-yl)amino)pentyl)carbamate (30.0 mg, 0.064 mmol) in MeOH (5 mL) was added 5% Pd/C (10.0 mg). The reaction mixture was stirred at rt under hydrogen atmosphere for 2 h. The reaction mixture was filtered and the filtrate was concentrated under reduced pressure. The residue was purified by silica gel chromatography to provide the desired product (25.0 mg, 83% yield). MS (ESI) m/z=474.7 [M+H]⁺.

The remaining steps were performed according to the procedures for preparing CPD-25 to provide the desired product (9.0 mg, 20% yield over 3 steps) as a yellow solid. MS (ESI) m/z=872.8 [M+H]⁺.

### Example 166. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-chloro-6-cyclopropylpyridin-2-yl)-6-(methylamino)nicotinamide (CPD-112)

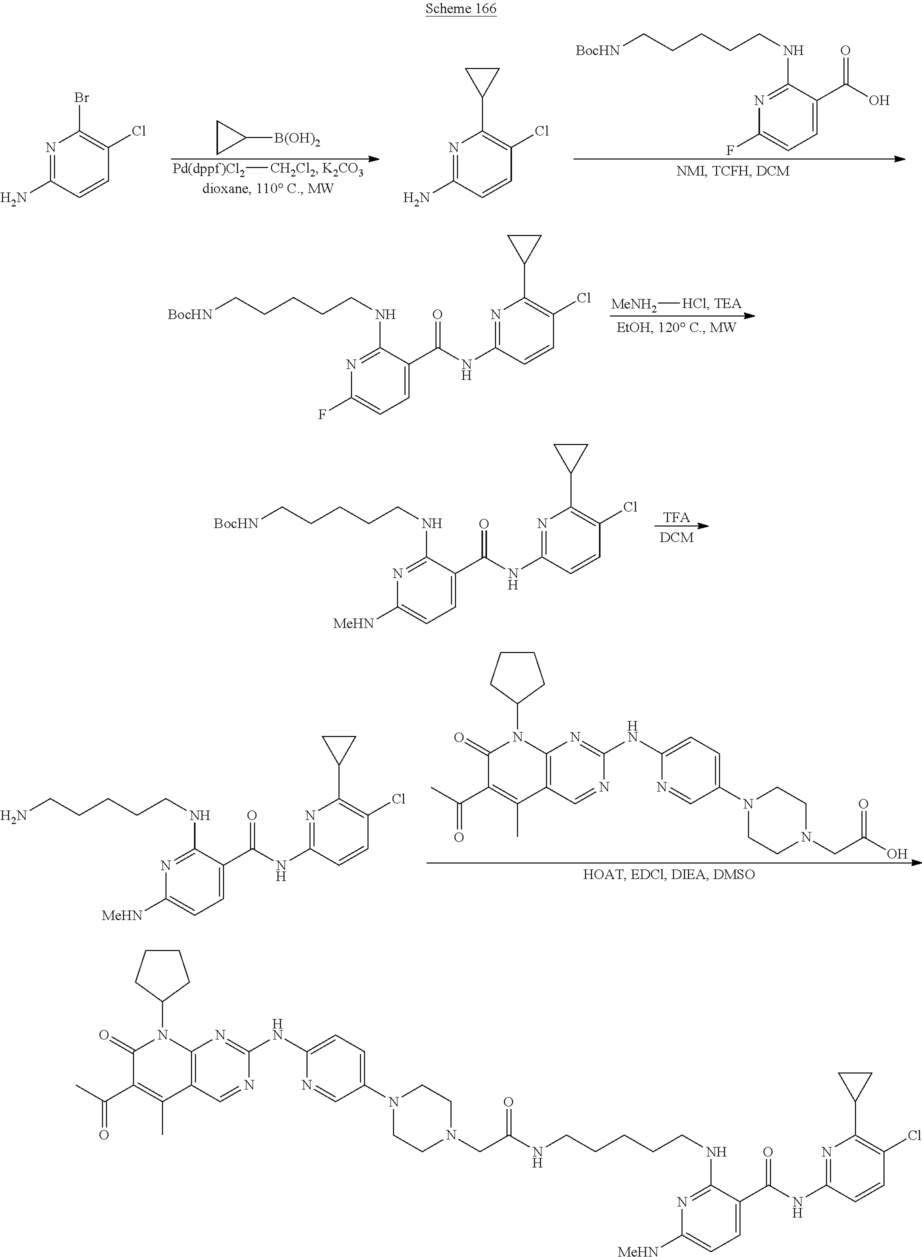

CPD-112 was synthesized following the procedures for preparing CPD-1 13 and CPD-31 (4.0 mg, 2% yield over 5 steps) as a yellow solid. MS (ESI) m/z=890.7 [M+H]⁺.

### Example 167. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-cyclopropyl-5-fluoropyridin-2-yl)-6-(methylamino)nicotinamide (CPD-113)

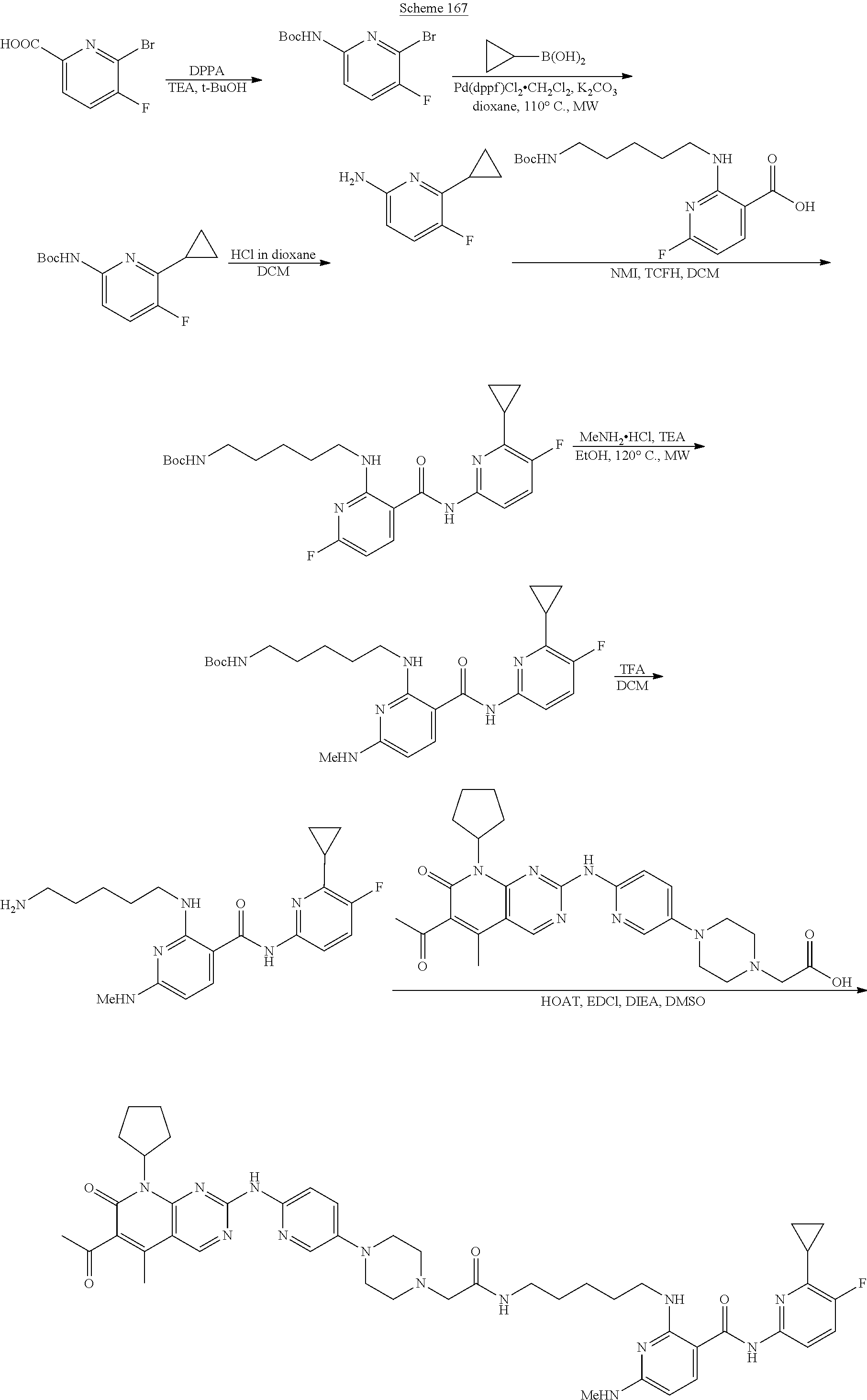

### Step 1. Synthesis of tert-butyl N-(6-bromo-5-fluoro-2-pyridyl)carbamate

To a solution of 6-bromo-5-fluoro-pyridine-2-carboxylic acid (1.00 g, 4.55 mmol) in tert-butanol (10 mL) were added DPPA (1.14 g, 4.15 mmol) and TEA (436 mg, 4.31 mmol) at rt. The reaction mixture was stirred at 70° C. for 2 h. After cooling down to rt, the reaction mixture was diluted with H₂O (30 mL) and extracted with EtOAc (10 mL×3). The combined organic layers were dried over Na₂SO₄, filtered, and concentrated under reduced pressure. The residue was purified by silica gel column chromatography (petroleum ether/ethyl acetate=30:1) to get the desired product (643 mg, 49% yield) as a colorless oil. ¹HNMR (400 MHz, CDCl₃) δ 7.89 (d, J=8.8 Hz, 1H), 7.34 (d, J=8.8 Hz, 1H), 1.44 (s, 9H).

### Step 2. Synthesis of tert-butyl N-(6-cyclopropyl-5-fluoro-2-pyridyl)carbamate

To a solution of cyclopropylboronic acid (900 mg, 10.5 mmol) and tert-butyl N-(6-bromo-5-fluoro-2-pyridyl)carbamate (643 mg, 2.21 mmol) in dioxane (8 mL) were added Pd(dppf)Cl₂ CH₂Cl₂ (350 mg, 0.43 mmol) and K₂CO₃ (450 mg, 3.26 mmol) at rt. The reaction mixture was stirred at 110° C. for 4 h under microwave irradiation. After cooling down to rt, the mixture was diluted with water (10 mL) and extracted with ethyl acetate (10 mL×3). The combined organic phase was washed with brine (10 mL×3), dried over anhydrous Na₂SO₄, filtered, and concentrated in vacuum. The residue was purified by prep-TLC (petroleum ether/ethyl acetate=3:1) to get the desired product (65.0 mg, 11% yield) as a yellow oil. MS (ESI) m/z=197.1 [M+H-55]⁺.

### Step 3. Synthesis of 6-cyclopropyl-5-fluoro-pyridin-2-amine

To a solution of tert-butyl N-(6-cyclopropyl-5-fluoro-2-pyridyl)carbamate (65.0 mg, 0.26 mmol) in DCM (1 mL) was added HCl solution (4 M in dioxane, 1 mL). The mixture was stirred at rt for 2 h. The reaction mixture was concentrated under vacuum to afford the desired product (16.7 mg, 310% yield) as a brown gum. ¹HNMR (400 MHz, DMSO-d₆) δ 7.80-7.51 (m, 1H), 6.59 (d, J=5.2 Hz, 1H), 2.17-2.13 (m, 1H), 1.16-1.02 (m, 4H). MS (ESI) m/z=153.4 [M+H]⁺.

The remaining steps were performed according to the procedures for preparing CPD-31 to provide the desired product (5.0 mg, 9% yield over 4 steps) as a yellow solid. MS (ESI) m/z=874.8 [M+H]⁺.

### Example 168. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-3-fluoropyridin-2-yl)-6-(methylamino)nicotinamide (CPD-114)

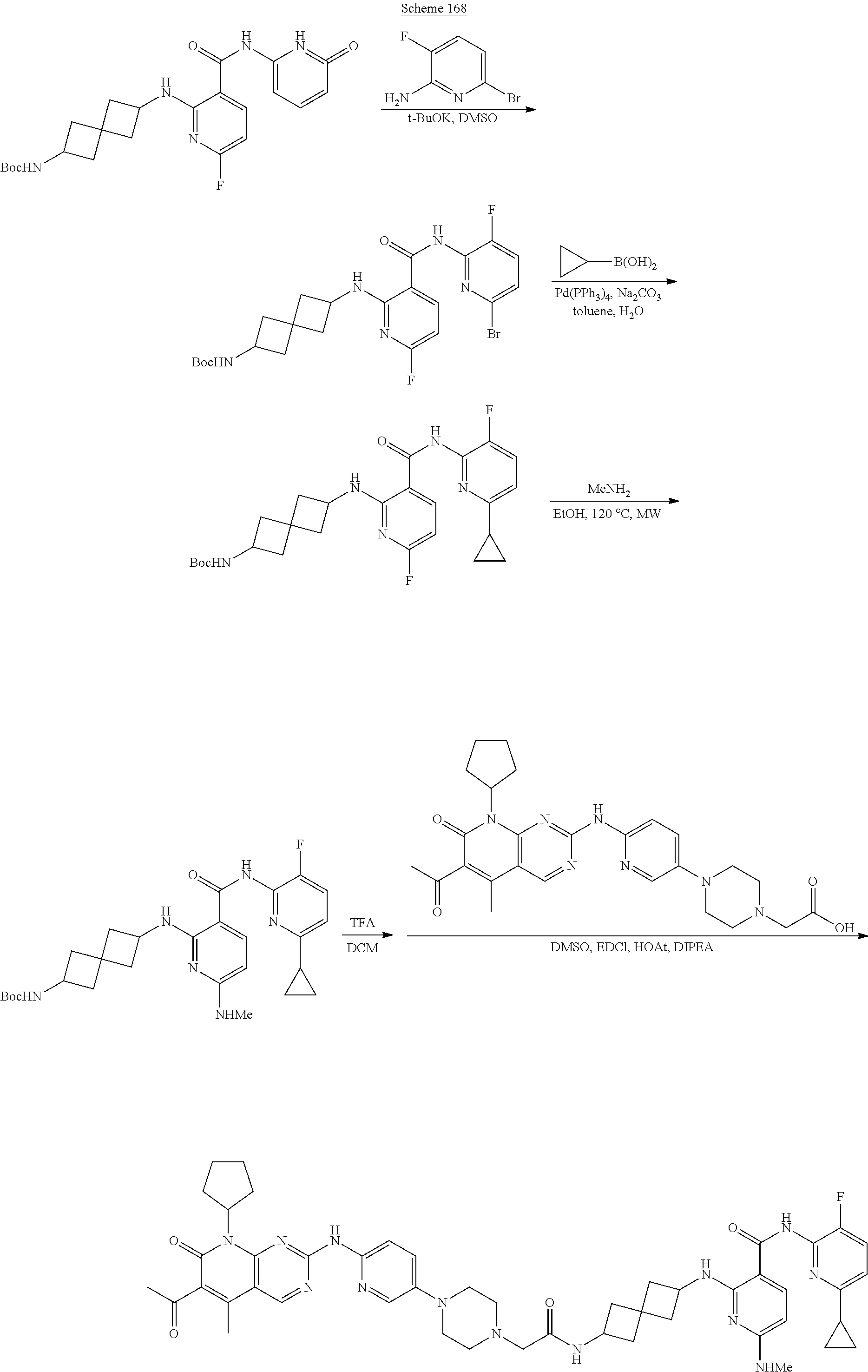

### Step 1. Synthesis of tert-butyl (6-((3-((6-Bromo-3-fluoropyridin-2-yl)carbamoyl)-6-fluoropyridin-2-yl)amino)spiro[3.3]heptan-2-yl)carbamate

To a mixture of tert-butyl (6-((6-fluoro-3-((6-oxo-1,6-dihydropyridin-2-yl)carbamoyl)pyridine-2-yl)amino)spiro[3.3]heptan-2-yl)carbamate (70 mg, 0.15 mmol) in DMSO (1 mL) was added t-BuOK (1 M in THF, 0.1 mL, 0.1 mmol) at rt. The reaction mixture was stirred at rt for 20 min. The reaction mixture was quenched with water (10 mL) and extracted with ethyl acetate (10 mL×3). The combined organic layers were dried over anhydrous Na₂SO₄, filtered, and concentrated in vacuum. The residue was purified by reverse- phase chromatography to provide the desired product (25 mg, 30% yield) as a pale-yellow solid. MS (ESI) m/z=538.4 [M+H]⁺.

### Step 2. Synthesis of tert-butyl (6-((3-((6-cyclopropyl-3-fluoropyridin-2-yl)carbamoyl)-6-fluoropyridin-2-yl)amino)spiro[3.3]heptan-2-yl)carbamate

A mixture of tert-butyl (6-((3-((6-Bromo-3-fluoropyridin-2-yl)carbamoyl)-6-fluoropyridin-2-yl)amino)spiro[3.3]heptan-2-yl)carbamate (25 mg, 0.046 mmol), cyclopropylboronic acid (12 mg, 0.14 mmol), Na₂CO₃ (24.6 mg, 0.23 mmol) and Pd(PPh₃)₄ (8 mg, 7.0 μmol) in toluene (3 mL) and water (0.6 mL) was stirred at 100° C. for 6 h under nitrogen atmosphere. After cooling down to rt, the reaction mixture was diluted with water and extracted with DCM. The combined organic layers were concentrated and purified by reverse-phase chromatography to provide the desired product (10 mg, 43% yield) as a yellow solid. MS (ESI) m/z=500.6 [M+H]⁺.

The remaining steps were performed according to the procedures for preparing CPD-25 to provide the desired product (0.9 mg, 5% yield over 3 steps) as a yellow solid. MS (ESI) m/z=898.8 [M+H]⁺.

### Example 169. 2-((2-(1-((4-(6-((6-Acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(6-(dimethylamino)pyridazin-3-yl)-6-(methylamino)nicotinamide (CPD-115)

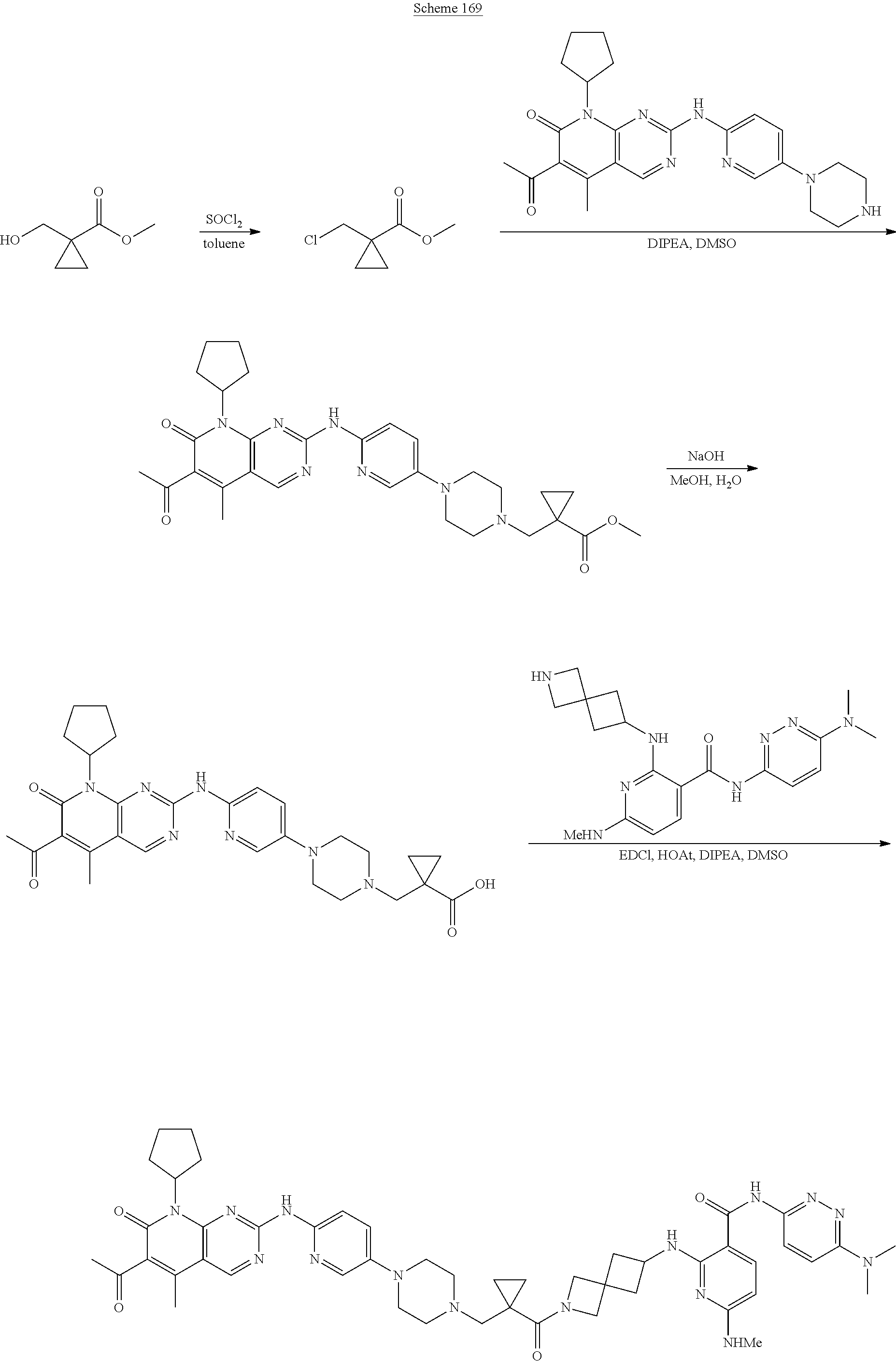

### Step 1. Synthesis of methyl 1-(chloromethyl)cyclopropane-1-carboxylate

A mixture of methyl 1-(hydroxymethyl)cyclopropanecarboxylate (100 mg, 0.77 mmol) and SOCl₂ (274 mg, 2.31 mmol) in toluene (1.0 mL) was stirred at 50° C. for 2 h. Then the reaction mixture was concentrated to afford the crude product (100 mg, 88% yield) as a white solid.

### Step 2. Synthesis of methyl 1-((4-(6-((6-acetyl-8-cyclopentyl-5-methyl-7-oxo-7,8-dihydro pyrido[2,3-d]pyrimidin-2-yl)amino)pyridin-3-yl)piperazin-1-yl)methyl)cyclopropane-1-carboxylate

A mixture of 6-acetyl-8-cyclopentyl-5-methyl-2-[(5-piperazin-1-yl-2-pyridyl)amino]pyrido[2,3-d]pyrimidin-7-one (60 mg, 0.13 mmol), DIPEA (104 mg, 0.8 mmol) and methyl 1-(chloromethyl)cyclopropane-1-carboxylate (60 mg, 0.4 mmol) in DMSO (1 mL) was stirred at 90° C. for 1.5 h. After cooling down to rt, the reaction mixture was purified by reverse-phase chromatography to afford the desired product (15 mg, 20% yield) as a pale-yellow solid. MS (ESI) m/z=560.6 [M+H]⁺.

### Step 3. Synthesis of 1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carboxylic acid

A mixture of methyl 1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carboxylate (15 mg, 0.027 mmol) and NaOH (4.3 mg, 0.11 mmol) in MeOH (1 mL) and water (1 mL) was stirred at 50° C. for 1 h. The reaction mixture was concentrated and purified by reverse-phase chromatography to afford the desired product (8 mg, 55% yield) as a yellow solid. MS (ESI) m/z=546.6 [M+H]⁺.

### Step 4. Synthesis of 2-((2-(1-((4-(6-((6-acetyl-8-cyclopentyl-5-methyl-7-oxo-7,8-dihydro pyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(6-(dimethylamino)pyridazin-3-yl)-6-(methylamino)nicotinamide

The title compound was synthesized following the procedure for preparing CPD-1 (0.95 mg, 10% yield) as a yellow solid. MS (ESI) m/z=910.7 [M+H]⁺.

### Example 170. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-methoxy-5-methylpyridin-2-yl)-6-(methylamino)nicotinamide (CPD-116)

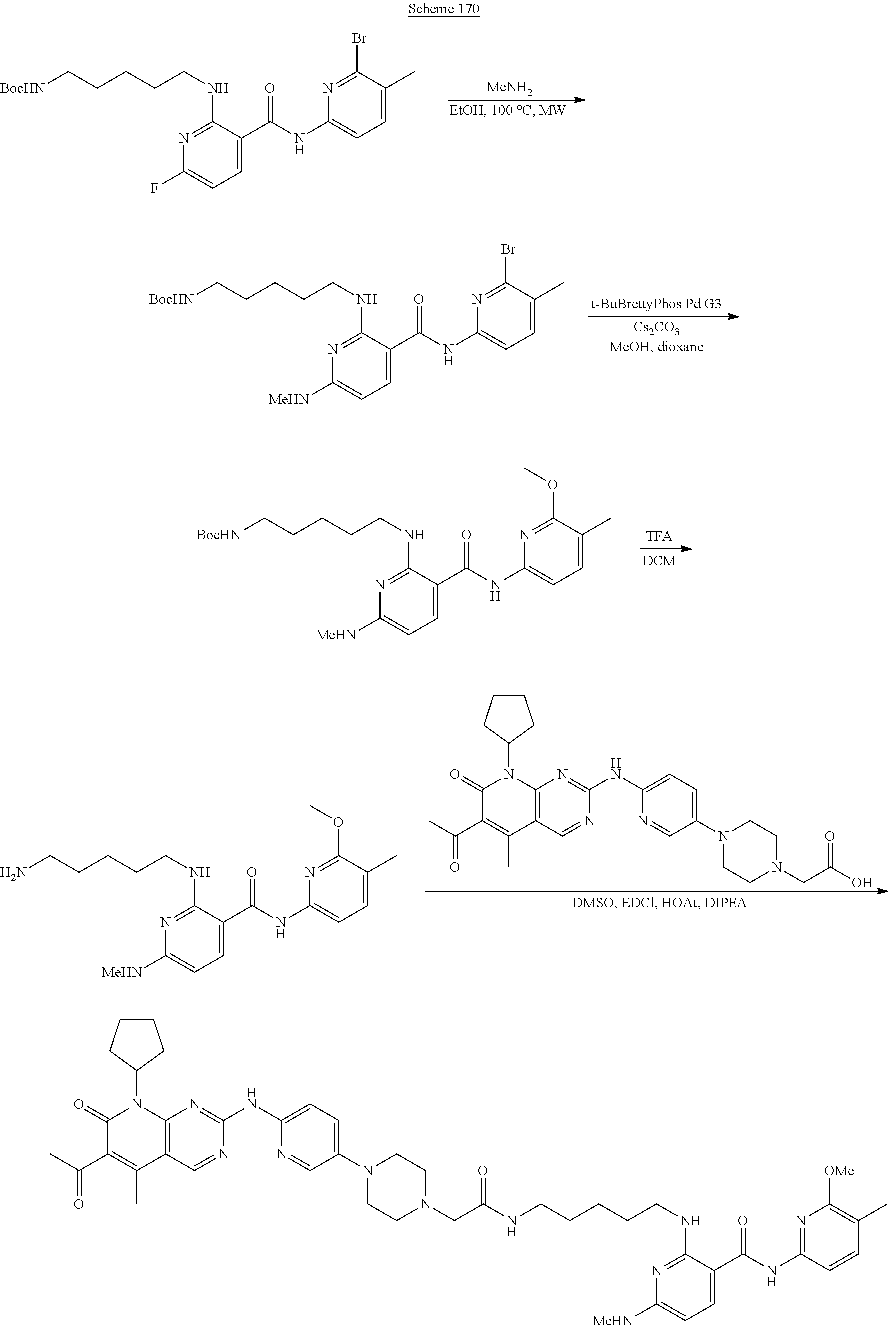

### Step 1. Synthesis of tert-butyl (5-((3-((6-bromo-5-methylpyridin-2-yl)carbamoyl)-6-(methylamino)pyridin-2-yl)amino)pentyl)carbamate

To a solution of tert-butyl (5-((3-((6-bromo-5-methylpyridin-2-yl)carbamoyl)-6-fluoropyridin-2-yl)amino)pentyl)carbamate (130.0 mg, 0.25 mmol) in EtOH (1 mL) was added MeNH₂ solution (1.0 mL, 30 wt % in EtOH) at rt. The reaction mixture was stirred at 100° C. under microwave irradiation for 40 min. After cooling down to rt, the reaction mixture was diluted with water (10 mL) and extracted with ethyl acetate (3×10 mL). The combined organic layers were washed with saturated brine (15 mL), dried over anhydrous sodium sulfate, filtered, and evaporated under reduced pressure. The resulting residue was purified by silica gel chromatography to provide the desired product (125.0 mg, 94% yield) as a pale-white solid. MS (ESI) m/z=521.4 [M+H]⁺.

### Step 2. Synthesis of tert-butyl (5-((3-((6-methoxy-5-methylpyridin-2-yl)carbamoyl)-6-(methylamino)pyridin-2-yl)amino)pentyl)carbamate

To a solution of tert-butyl (5-((3-((6-bromo-5-methylpyridin-2-yl)carbamoyl)-6-(methylamino)pyridin-2-yl)amino)pentyl)carbamate (125.0 mg, 0.24 mmol) in MeOH (5 mL) and dioxane (5 mL) were added t-BuBrettPhos Pd G3 (20.5 mg, 0.024 mmol), Cs₂CO₃ (156.3 mg, 0.48 mmol) at rt. The reaction mixture was stirred at 100° C. under inert atmosphere for 16 h. After cooling down to rt, the reaction mixture was quenched with water (50 mL) and extracted with ethyl acetate (3×50 mL). The combined organic layers were washed with saturated brine (50 mL), dried over anhydrous sodium sulfate, filtered, and evaporated under reduced pressure. The resulting residue was purified by silica gel chromatography to provide the desired product (79.0 mg, 70% yield) as a pale-white solid. MS (ESI) m/z=473.7 [M+H]⁺.

The remaining steps were performed according to the procedures for preparing CPD-25 to provide the desired product (2.0 mg, 4% yield over 2 steps) as a light-yellow solid. MS (ESI) m/z=860.8 [M+H]⁺.

### Example 171. 2-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2-methylpropanoyl)-2-azaspiro[3.3]heptan-6-yl)amino)-N-(6-(dimethylamino)pyridazin-3-yl)-6-(methylamino)nicotinamide (CPD-117)

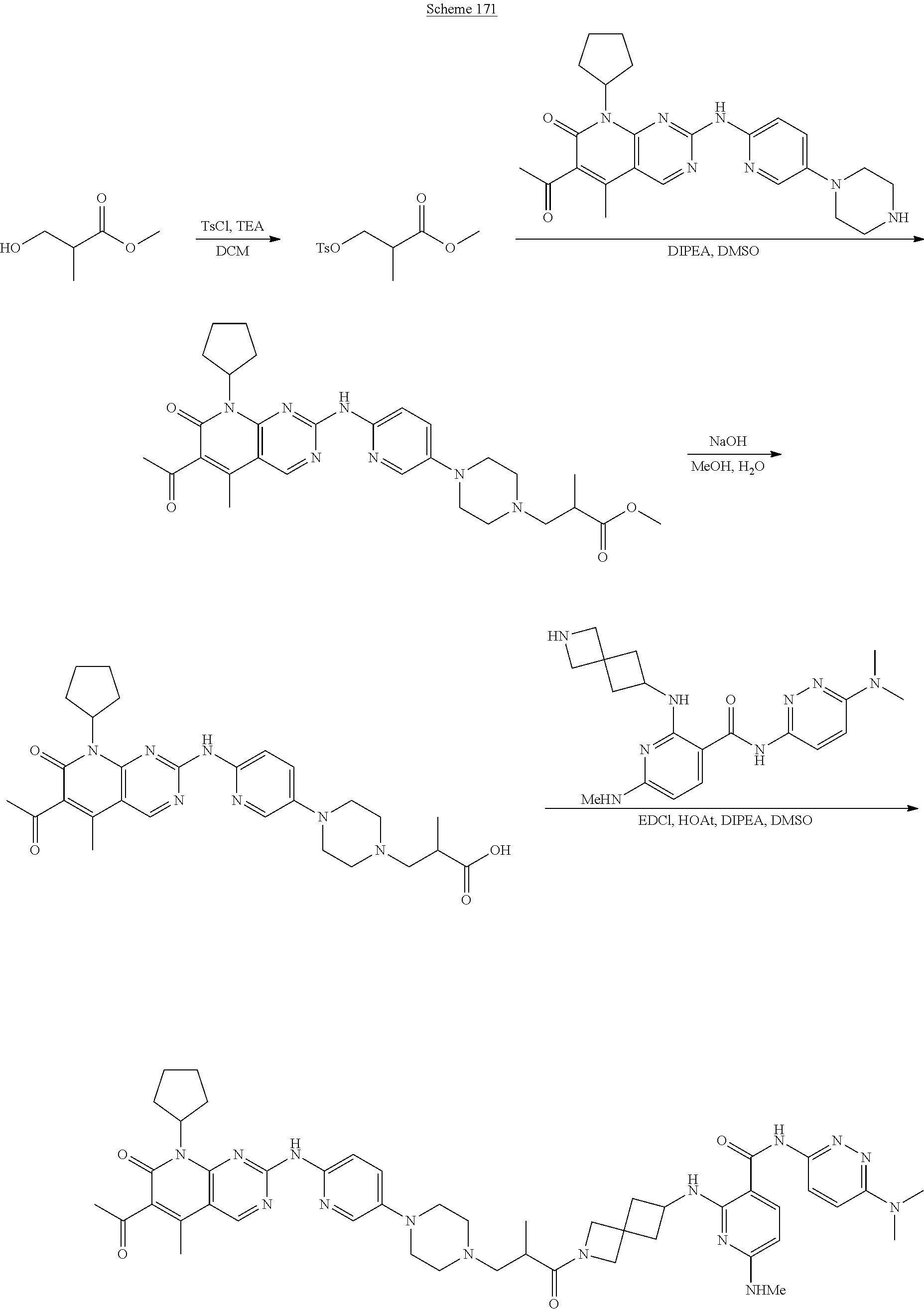

### Step 1. Synthesis of methyl 2-methyl-3-(tosyloxy)propanoate

To a solution of methyl 3-hydroxy-2-methylpropanoate (200 mg, 1.69 mmol) in DCM (10 mL) were added TsCl (321.1 mg, 1.69 mmol) and TEA (513.1 mg, 5.08 mmol) at rt. The reaction mixture was stirred at rt for 16 h. The reaction mixture was poured into water (20 mL) and extracted with DCM (3×20 mL). The combined organic layers were washed with saturated brine, dried over anhydrous sodium sulfate, filtered, and evaporated under reduced pressure. The resulting residue was purified by reverse-phase chromatography to provide the desired product (220 mg, 4800 yield) as a colorless oil. MS (ESI) m/z=273.2 [M+H]⁺.

The remaining steps were performed according to the procedures for preparing CPD-115 to provide the desired product (6.0 mg, 4% yield over 3 steps) as a yellow solid. MS (ESI) m/z=898.8 [M+H]⁺.

### Example 172. 2-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)-2,2-dimethylpropanoyl)-2-azaspiro[3.3]heptan-6-yl)amino)-N-(6-(dimethylamino)pyridazin-3-yl)-6-(methylamino)nicotinamide (CPD-118)

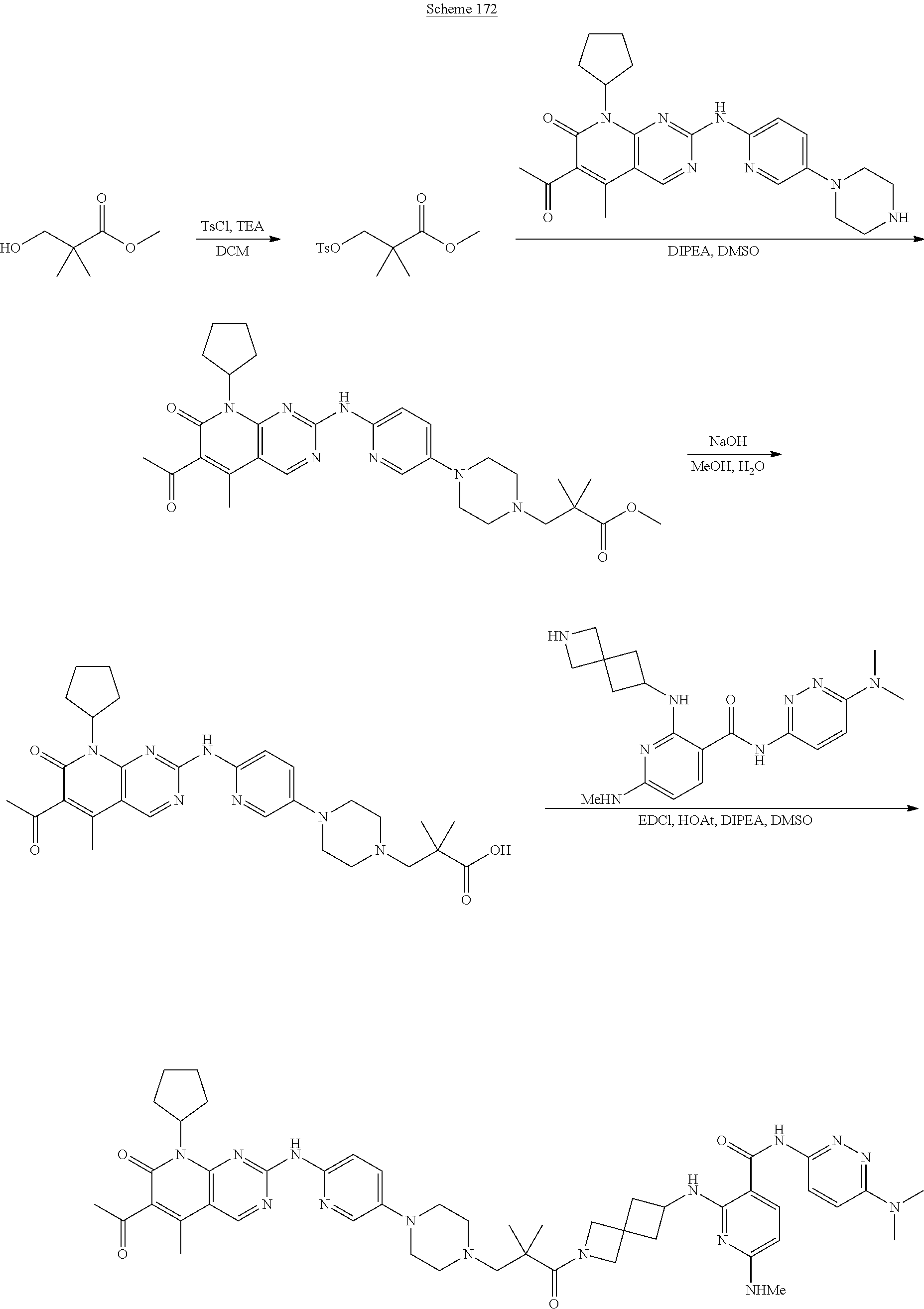

CPD-118 was synthesized following the procedures for preparing CPD-1 17 and CPD-1 15 (0.73 mg, 1% yield over 4 steps) as a yellow solid. MS (ESI) m/z=912.9 [M+H]⁺.

### Example 173. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-isopropylnicotinamide (CPD-119)

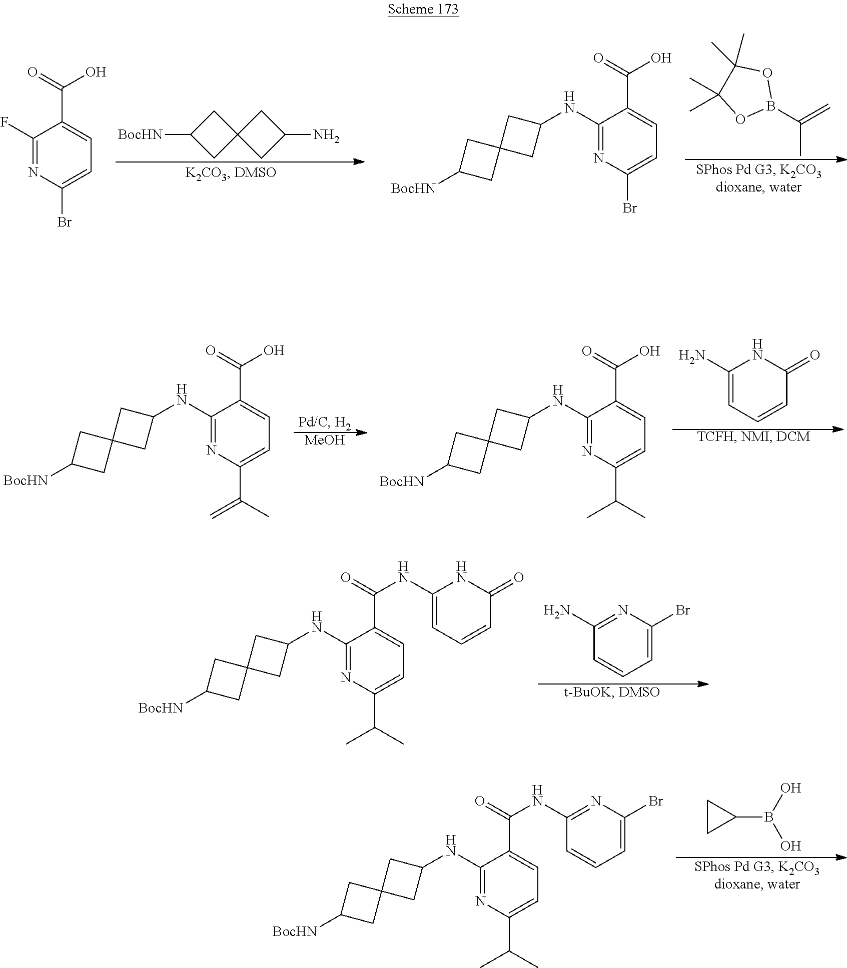

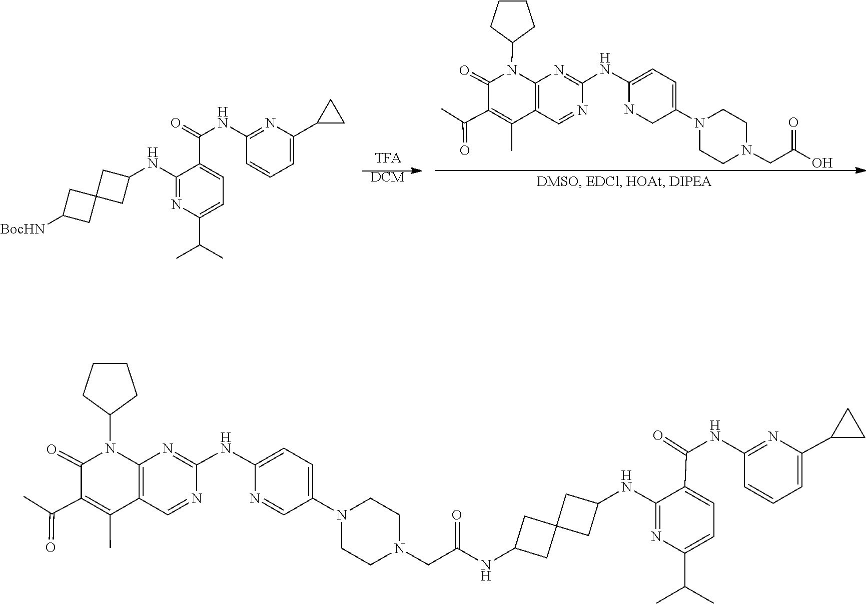

### Step 1. Synthesis of 6-bromo-2-((6-((tert-butoxycarbonyl)amino)spiro[3.3]heptan-2-yl)amino)nicotinic acid

To a mixture of 6-bromo-2-fluoronicotinic acid (500.0 mg, 2.27 mmol) and tert-butyl (6-aminospiro[3.3]heptan-2-yl)carbamate (565.80 mg, 2.50 mmol) in DMSO (10 mL) was added K₂CO₃ (627.3 mg, 4.55 mmol) at rt. The reaction mixture was stirred at 100 [symbol] for 16 h. After cooling down to rt, the reaction mixture was quenched with water (100 mL) and extracted with ethyl acetate (3×30 mL). The combined organic layers were washed with saturated brine (60 mL), dried over anhydrous sodium sulfate, filtered, and evaporated under reduced pressure. The resulting residue was purified by silica gel chromatography to give the desired product (700.0 mg, 72% yield) as a pale-white solid. MS (ESI) m/z=426.3 [M+H]⁺.

### Step 2. Synthesis of 2-((6-((tert-butoxycarbonyl)amino)spiro[3.3]heptan-2-yl)amino)-6-(prop-1-en-2-yl)nicotinic acid

To a solution of 6-bromo-2-((6-((tert-butoxycarbonyl)amino)spiro[3.3]heptan-2-yl)amino)nicotinic acid (100.0 mg, 0.23 mmol) in dioxane (5 mL) and water (1 mL) were added 4,4,5,5-tetramethyl-2-(prop-1-en-2-yl)-1,3,2-dioxaborolane (39.42 mg, 0.23 mmol), SPhos Pd G3 (20.3 mg, 0.023 mmol) and K₂CO₃ (64.7 mg, 0.47 mmol) at rt. The reaction mixture was stirred at 100 [symbol] under inert atmosphere for 16 h. After cooling down to rt, the reaction mixture was diluted with water (50 mL) and extracted with ethyl acetate (3×20 mL). The combined organic layers were washed with saturated brine (30 mL), dried over anhydrous sodium sulfate, filtered, and evaporated under reduced pressure. The resulting residue was purified by silica gel chromatography to provide the desired product (80.0 mg, 88% yield) as a pale-white solid. MS (ESI) m/z=388.7 [M+H]⁺.

### Step 3. Synthesis of 2-((6-((tert-butoxycarbonyl)amino)spiro[3.3]heptan-2-yl)amino)-6-isopropylnicotinic acid

To a solution of 2-((6-((tert-butoxycarbonyl)amino)spiro[3.3]heptan-2-yl)amino)-6-(prop-1-en-2-yl)nicotinic acid (80.0 mg, 0.21 mmol) in MeOH (5 mL) was added 5% Pd/C (10.0 mg). The reaction mixture was stirred at rt under hydrogen atmosphere for 2 h. The reaction mixture was filtered and concentrated to give the crude. The residue was purified by silica gel chromatography to provide the desired product (75.0 mg, 93% yield). MS (ESI) m/z=390.8 [M+H]⁺.

The remaining steps were performed according to the procedures for preparing CPD-111 to provide the desired product (1.2 mg, 1% yield over 5 steps) as a yellow solid. MS (ESI) m/z=893.8 [M+H]⁺.

### Example 174. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-methylnicotinamide (CPD-120)

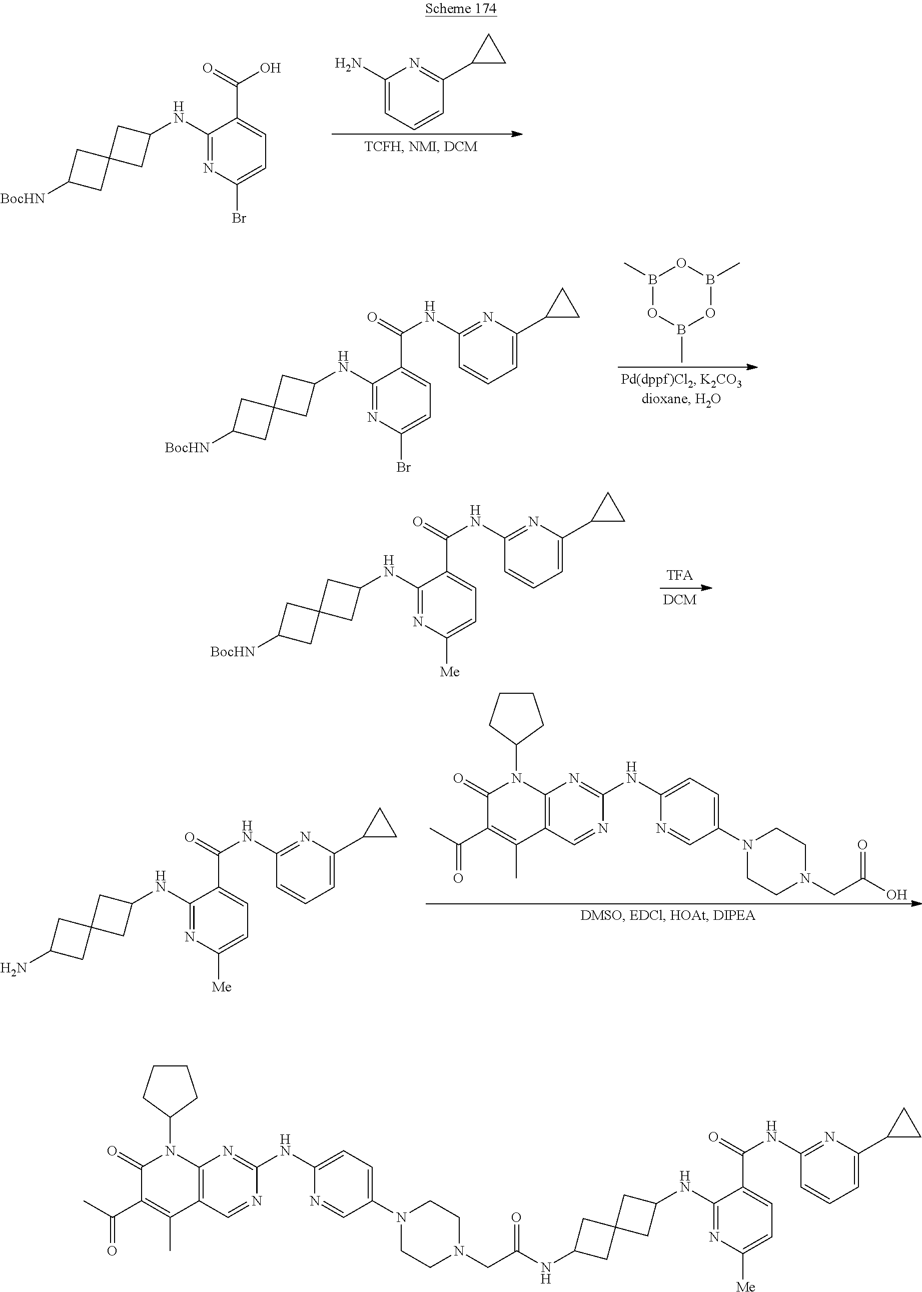

CPD-120 was synthesized following the procedures for preparing CPD-45 (5.6 mg, 13% yield over 4 steps) as a yellow solid. MS (ESI) m/z 865.9 [M+H]⁺.

### Example 175. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(trifluoromethyl)nicotinamide (CPD-121)

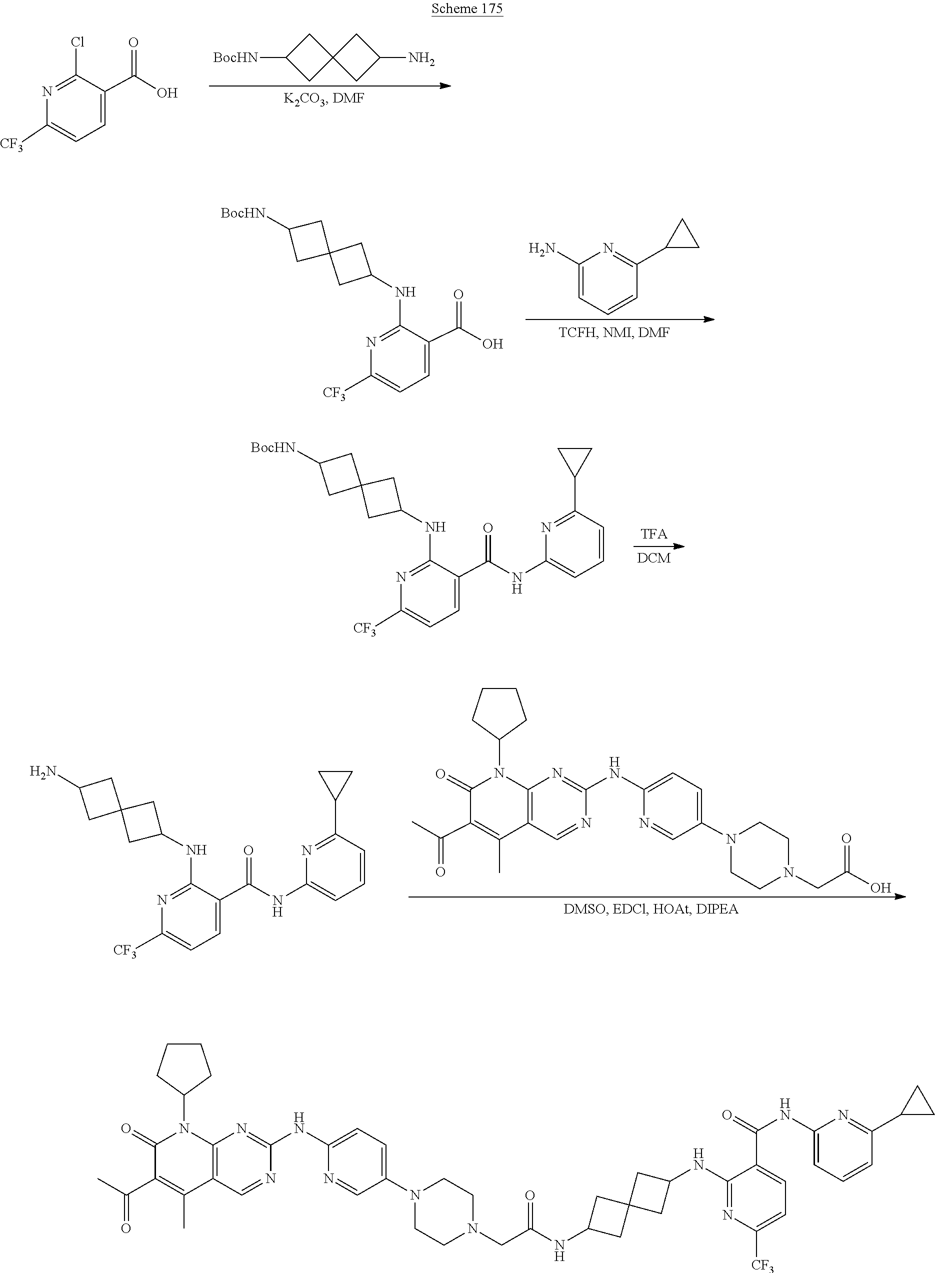

CPD-121 was synthesized following the procedures for preparing CPD-39 and CPD-31 (2.0 mg, 1% yield over 4 steps) as a yellow solid. MS (ESI) m/z=919.9 [M+H]⁺.

### Example 176. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-oxo-1,6-dihydropyridin-2-yl)nicotinamide (CPD-122)

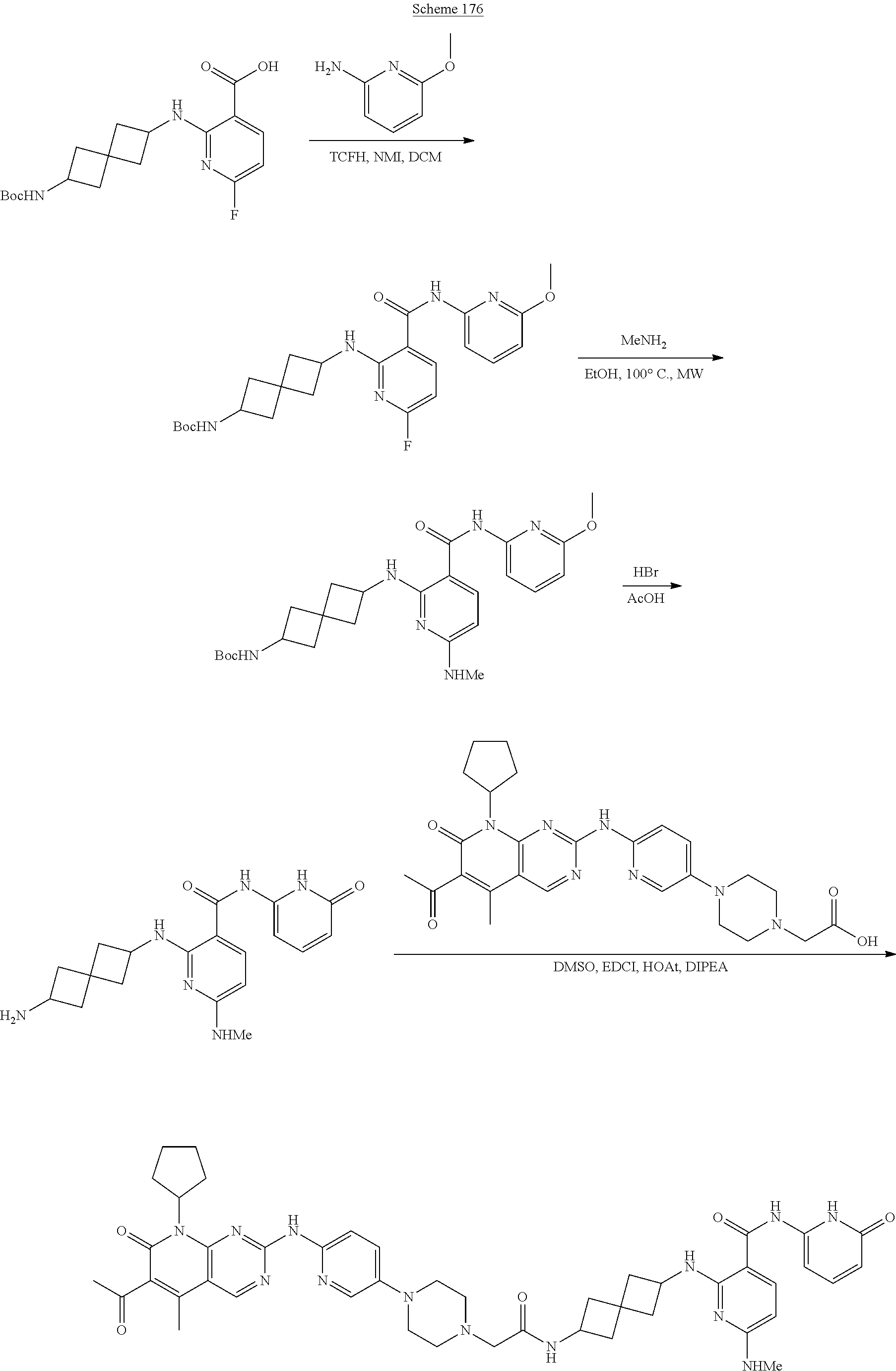

### Step 1 to 2. Synthesis of tert-butyl (6-((3-((6-methoxypyridin-2-yl)carbamoyl)-6-(methylamino)pyridin-2-yl)amino)spiro[3.3]heptan-2-yl)carbamate

The title compound was synthesized following the procedures for preparing CPD-31 (83 mg, 64% yield over 2 steps) as a pale-white solid. MS (ESI) m/z=483.8 [M+H]⁺.

### Step 3. Synthesis of 2-((6-aminospiro[3.3]heptan-2-yl)amino)-6-(methylamino)-N-(6-oxo-1,6-dihydropyridin-2-yl)nicotinamide

A solution of tert-butyl (6-((3-((6-methoxypyridin-2-yl)carbamoyl)-6-(methylamino)pyridin-2-yl)amino)spiro[3.3]heptan-2-yl)carbamate (83.0 mg, 0.17 mmol) in HBr solution (4.0 mL, 33 wt % in AcOH) was stirred at 100° C. for 3 h. After cooling down to rt, the reaction mixture was poured into water (30 mL), neutralized with saturated sodium bicarbonate solution, and extracted with ethyl acetate (3×20 mL). The combined organic layers were washed with saturated brine (15 mL), dried over anhydrous sodium sulfate, filtered, and evaporated under reduced pressure. The resulting residue was purified by reverse-phase chromatography to provide the desired product (45.0 mg, 71% yield) as a pale-white solid. MS (ESI) m/z=369.5 [M+H]⁺.

### Step 4. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-oxo-1,6-dihydropyridin-2-yl)nicotinamide

The title compound was synthesized following the procedure for preparing CPD-1 to provide the desired product (3.1 mg, 3% yield) as a light yellow solid. MS (ESI) m/z=856.7 [M+H]⁺.

### Example 177. 2-((5-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(5-methyl-6-oxo-1,6-dihydropyridin-2-yl)-6-(methylamino)nicotinamide (CPD-123)

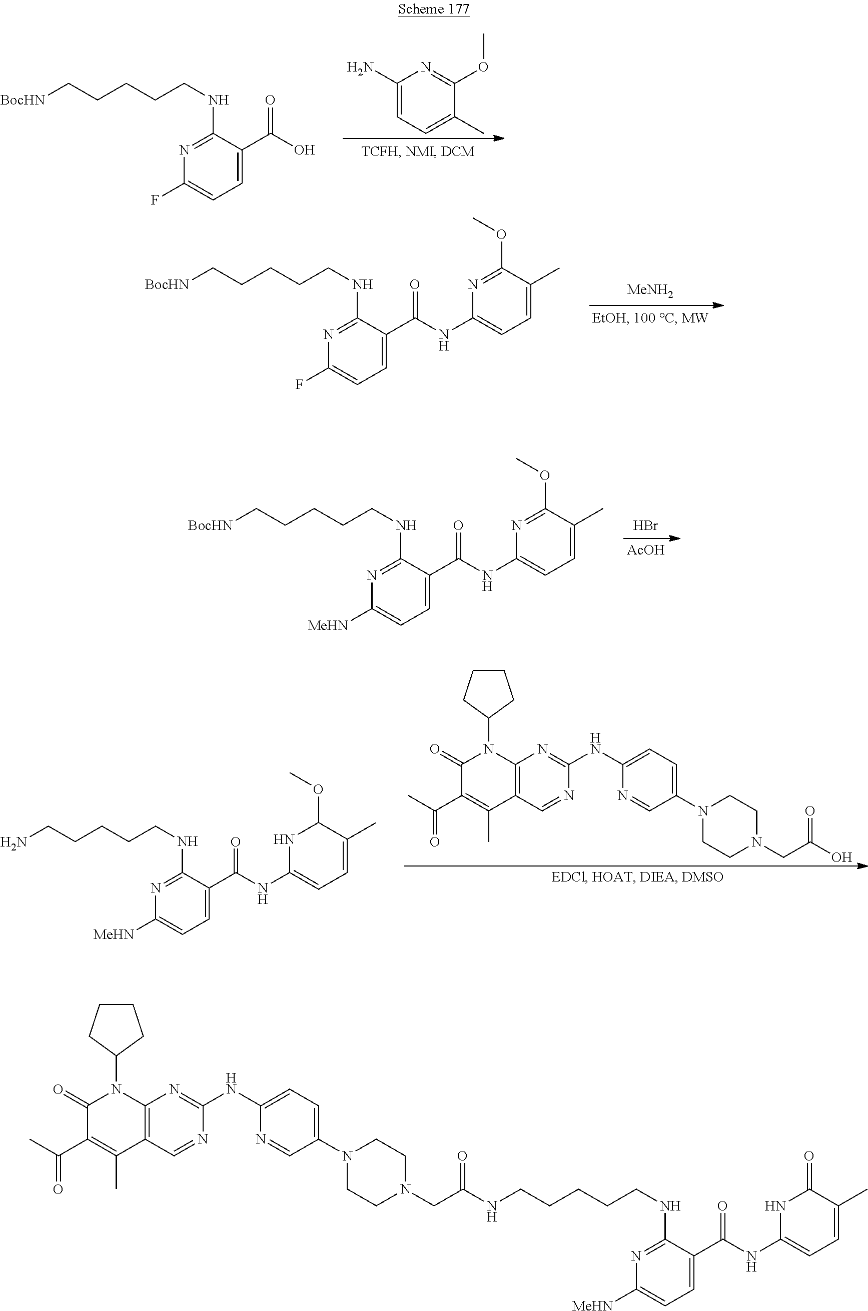

CPD-123 was synthesized following the procedures for preparing CPD-31 and CPD-122 (9.5 mg, 10% yield over 4 steps) as a yellow solid. MS (ESI) m/z=846.8 [M+H]⁺.

### Example 178. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-ethylnicotinamide (CPD-124)

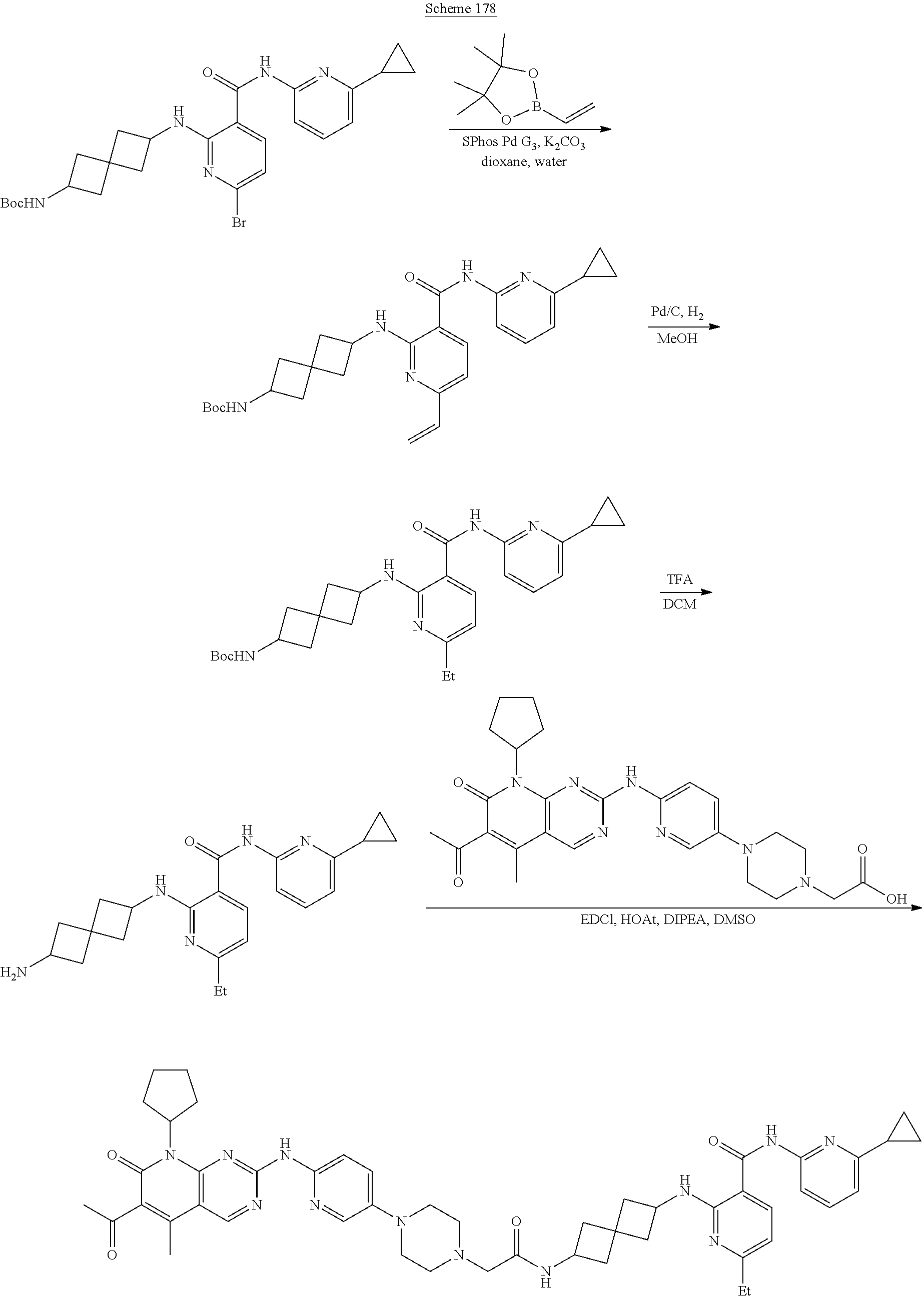

CPD-124 was synthesized following the procedures for preparing CPD-1 19 (10.1 mg, 18% o yield over 4 steps) as a yellow solid. MS (ESI) m/z=879.7 [M+H]⁺.

### Example 179. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-methoxypyridin-2-yl)-6-(methylamino)nicotinamide (CPD-125)

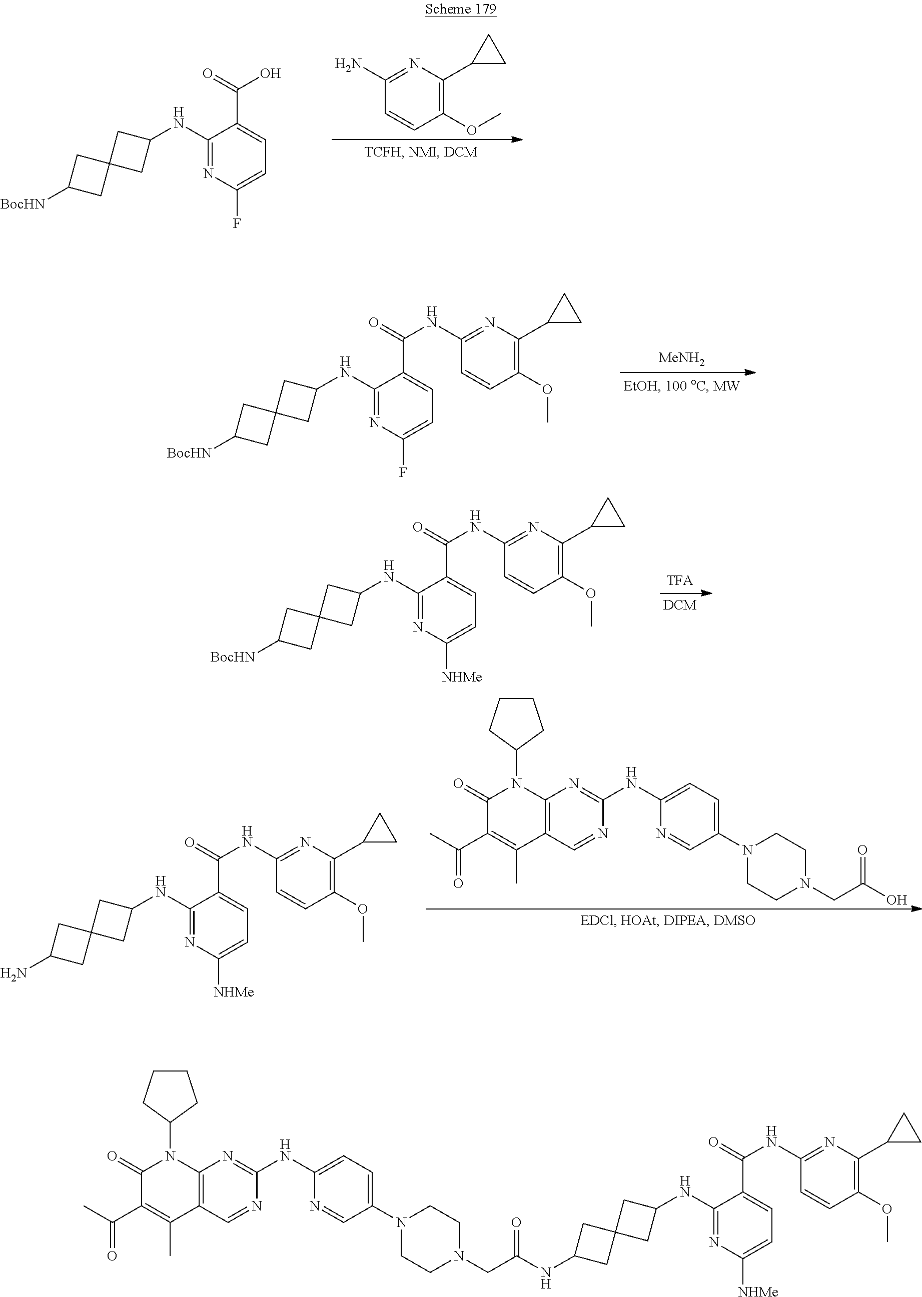

CPD-125 was synthesized following the procedures for preparing CPD-31 (3.6 mg, 5% yield over 4 steps) as a yellow solid. MS (ESI) m/z=910.8 [M+H]⁺.

### Example 180. 2-(((trans-4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-6-(methylamino)-N-(6-methylpyridin-2-yl)nicotinamide (CPD-126)

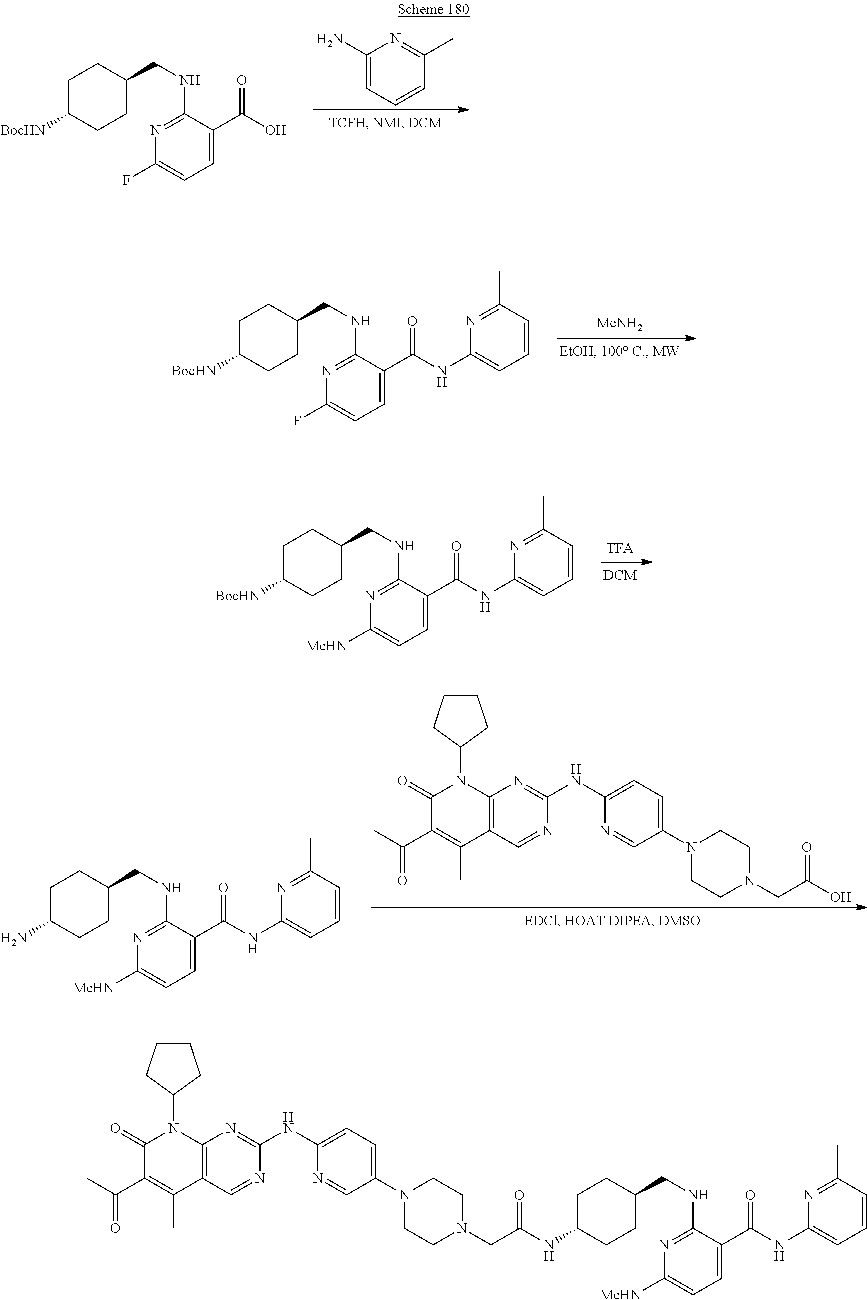

CPD-126 was synthesized following the procedures for preparing CPD-31 (0.91 mg, 2% yield over 4 steps) as a yellow solid. MS (ESI) m/z=856.8 [M+H]⁺.

### Example 181. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-5-(trifluoromethyl)-1H-pyrazol-3-yl)-6-(methylamino)nicotinamide (CPD-127)

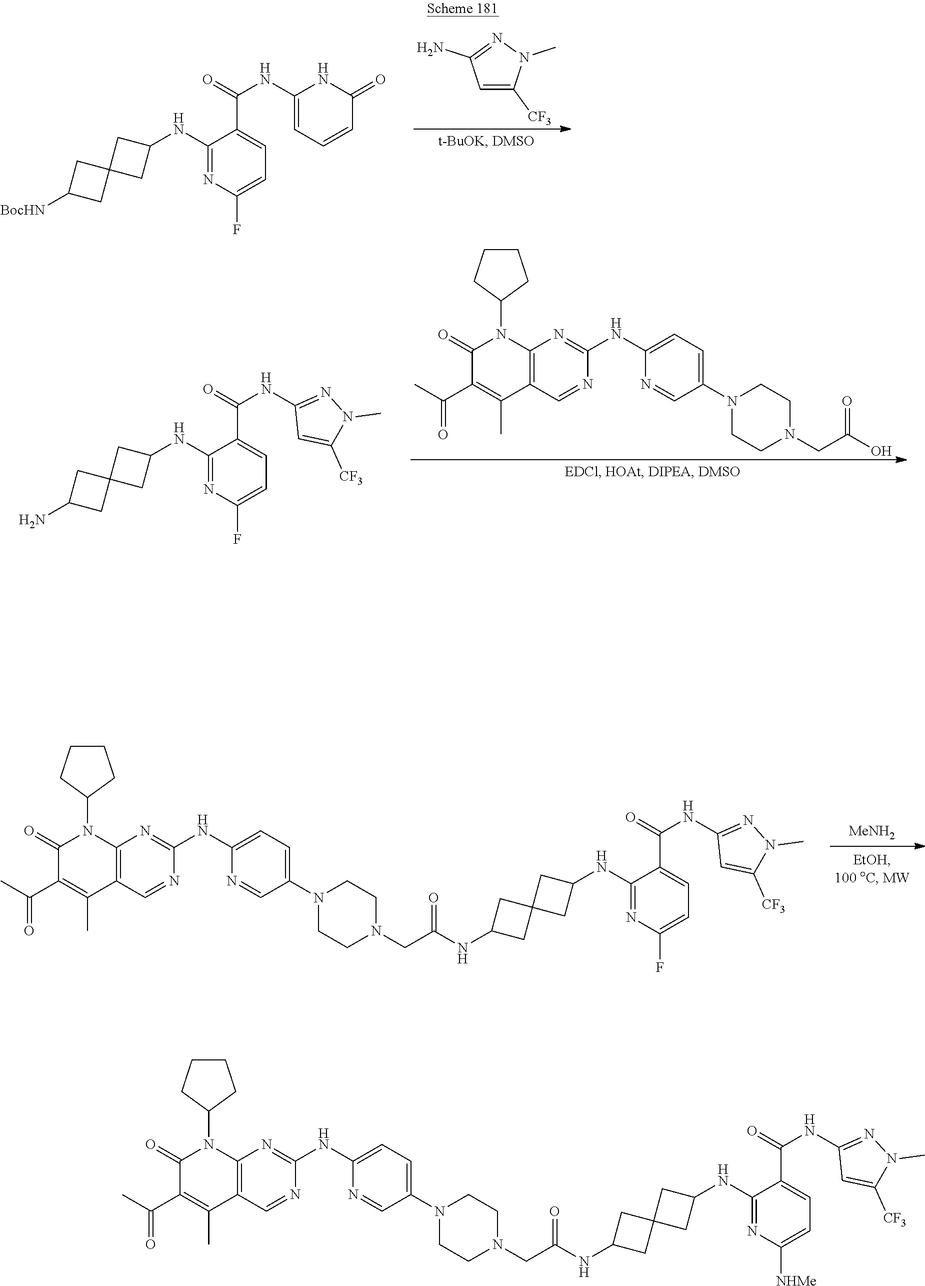

### Step 1. Synthesis of 2-((6-aminospiro[3.3]heptan-2-yl)amino)-6-fluoro-N-(1-methyl-5-(trifluoromethyl)-1H-pyrazol-3-yl)nicotinamide

To a solution of tert-butyl (6-((6-fluoro-3-((6-oxo-1,6-dihydropyridin-2-yl)carbamoyl)pyridin-2-yl)amino)spiro[3.3]heptan-2-yl)carbamate (40 mg, 87.4 mmol) and 1-methyl-5-(trifluoromethyl)-1H-pyrazol-3-amine (21.6 mg, 0.13 mmol) in DMSO (2 mL) was added t-BuOK (1 M in THF, 350 mL, 0.35 mmol). The reaction mixture was stirred at rt for 12 h. The mixture was purified by pre-HPLC to provide the desired product (8 mg, 22% yield) as a yellow oil. MS (ESI) m/z=413.1 [M+H]⁺.

### Step 2. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-fluoro-N-(1-methyl-5-(trifluoromethyl)-1H-pyrazol-3-yl)nicotinamide

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (12.7 mg, 0.025 mmol) in DMSO (2 mL) were added 2-((6-aminospiro[3.3]heptan-2-yl)amino)-6-fluoro-N-(1-methyl-5-(trifluoromethyl)-1H-pyrazol-3-yl)nicotinamide (8 mg, 0.019 mmol), EDCl (4 mg, 0.029 mmol), HOAt (5.6 mg, 0.029 mmol) and DIPEA (0.1 mL). The reaction mixture was stirred at rt for 12 h. The mixture was quenched with H₂O (10 mL), extracted with DCM (20 mL×3). The organic phase was washed with brine, dried with anhydrous Na₂SO₄, filtered, and concentrated under vacuum. The residue was purified by silica gel chromatography to provide the desired product (10 mg, 57% yield) as a yellow solid. MS (ESI) m/z=900.8 [M+H]⁺.

### Step 3. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-5-(trifluoromethyl)-1H-pyrazol-3-yl)-6-(methylamino)nicotinamide

A mixture of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-fluoro-N-(1-methyl-5-(trifluoromethyl)-1H-pyrazol-3-yl)nicotinamide (10 mg, 0.011 mmol) and methylamine (30% in EtOH, 0.2 mL) in EtOH (1 mL) was stirred at 100° C. for 1 h under microwave irradiation. After cooling down to rt, the reaction was quenched with H₂O (10 mL), extracted with DCM (20 mL×3). The organic phase was washed with brine, dried over anhydrous Na₂SO₄, filtered, and concentrated. The residue was purified by silica gel chromatography to provide the desired product (1.8 mg, 17% yield) as a yellow solid. MS (ESI) m/z=911.8 [M+H]⁺.

### Example 182. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-fluoropyridin-2-yl)-6-(methylamino)nicotinamide (CPD-128)

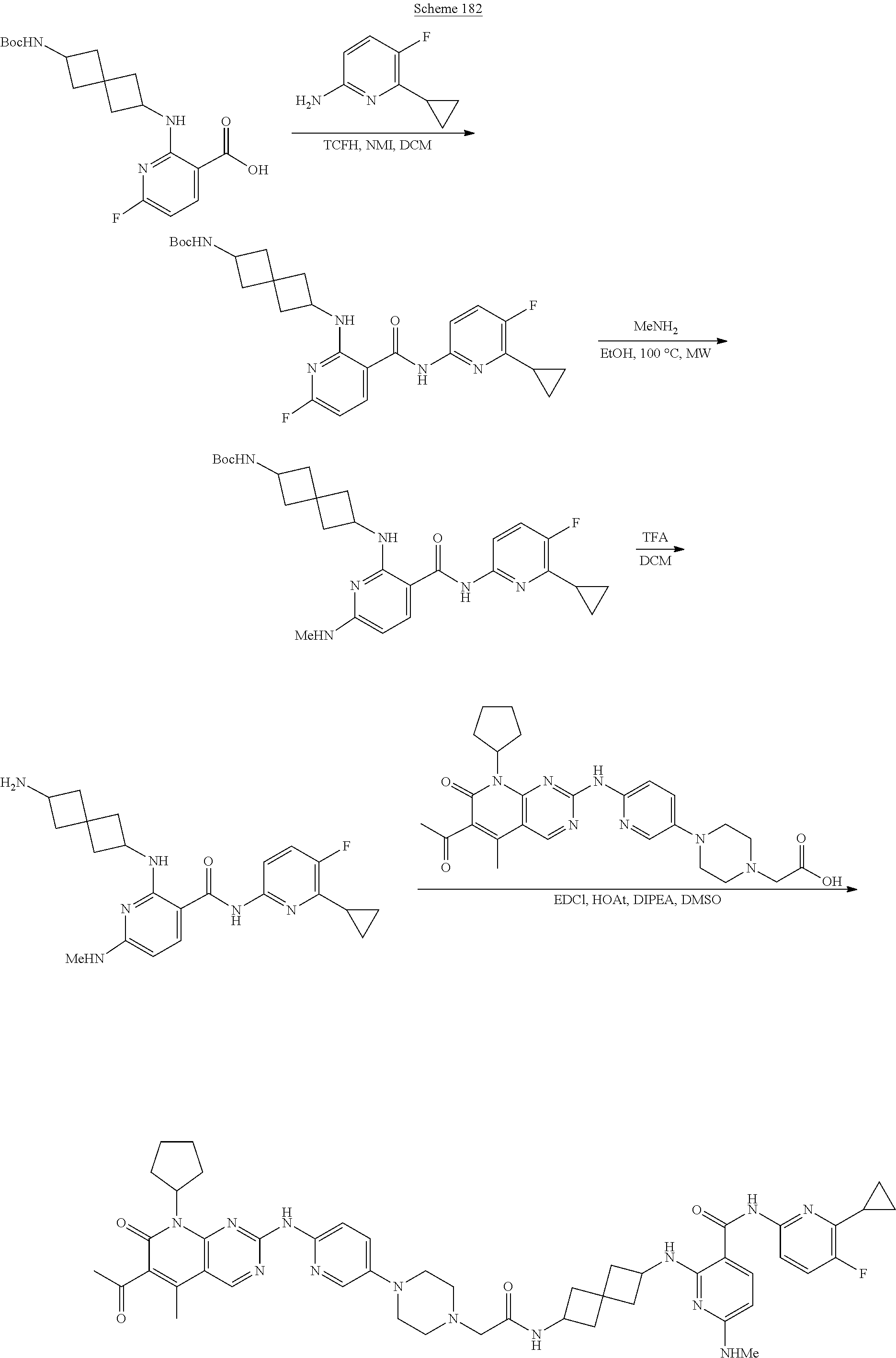

CPD-128 was synthesized following the procedures for preparing CPD-31 (5.1 mg, 4% yield over 4 steps) as a yellow solid. MS (ESI) m/z=898.8 [M+H]⁺.

### Example 183. 2-((2-(3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1,5-dimethyl-1H-pyrazol-3-yl)-6-(methylamino)nicotinamide (CPD-129)

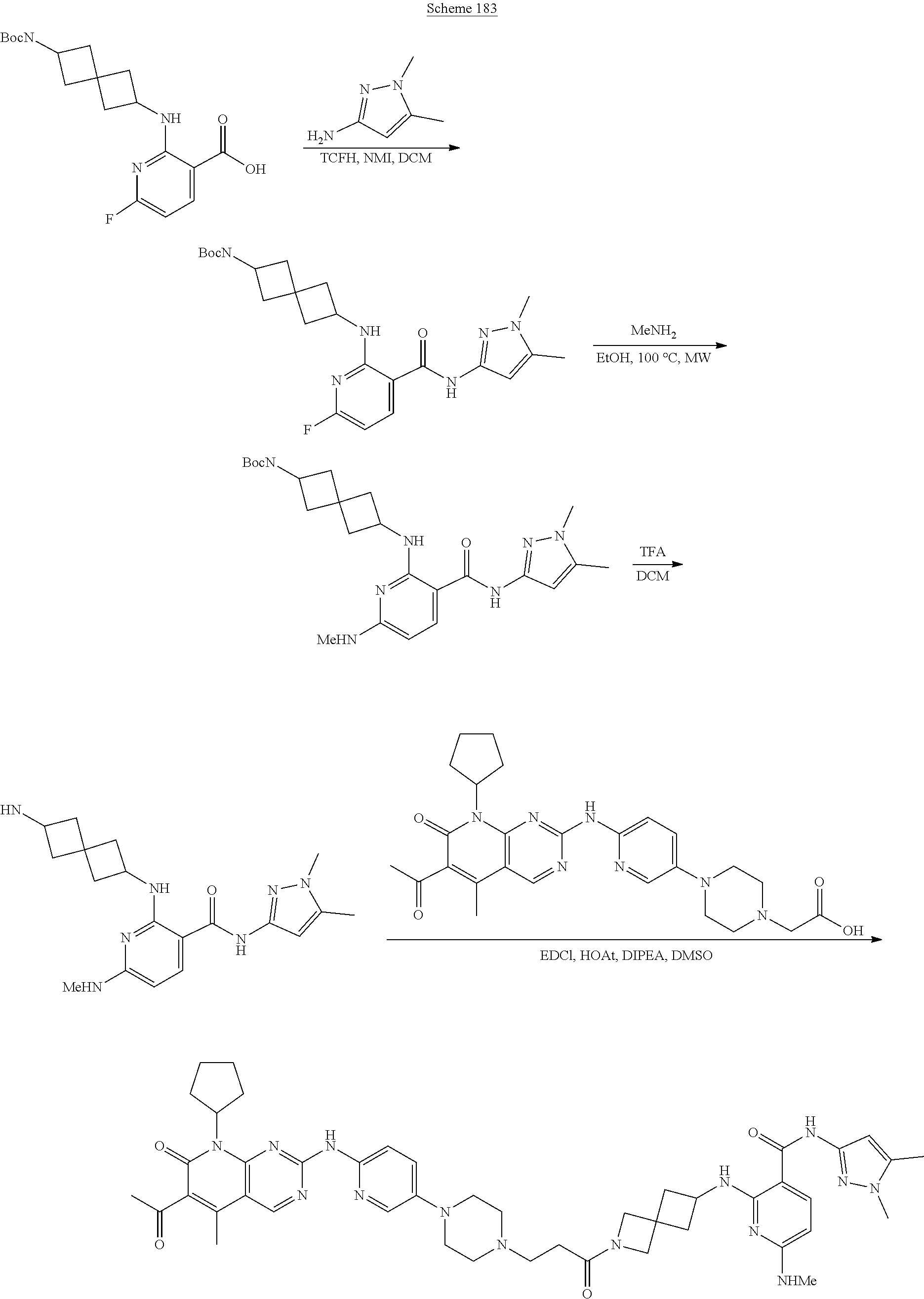

CPD-129 was synthesized following the procedures for preparing CPD-31 (12.5 mg, 11% yield over 4 steps) as a yellow solid. MS (ESI) m/z=857.8 [M+H]⁺.

### Example 184. 2-((4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)amino)-N-(6-(dimethylamino)pyridazin-3-yl)-6-(methylamino)nicotinamide (CPD-130)

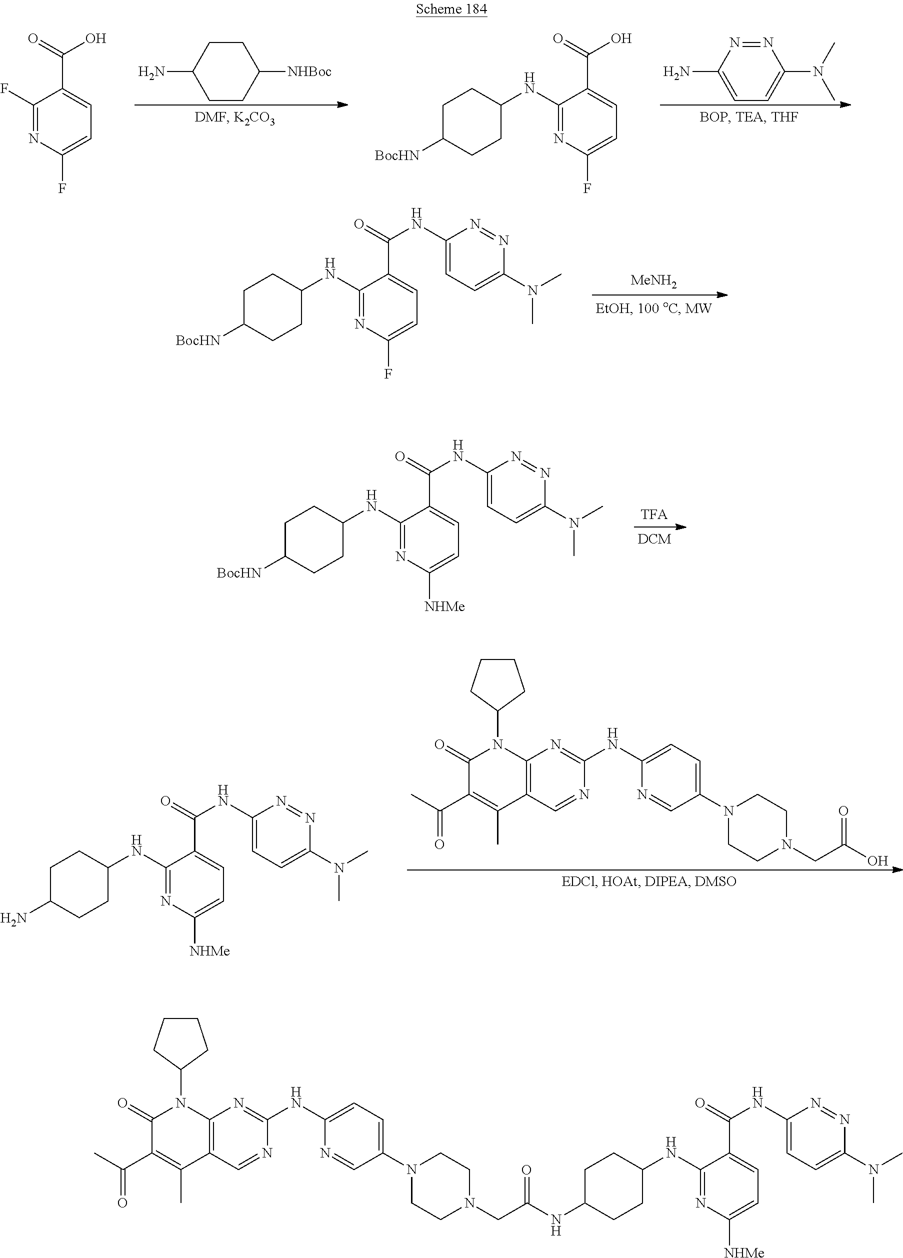

CPD-130 was synthesized following the procedures for preparing CPD-81 and CPD-25 (5.1 mug, 7% yield over 5 steps) as a yellow solid. MS (ESI) m/z=872.8 [M+H]⁺

### Example 185. 2-(((trans-4-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(1,5-dimethyl-1H-pyrazol-3-yl)-6-(methylamino)nicotinamide (CPD-131)

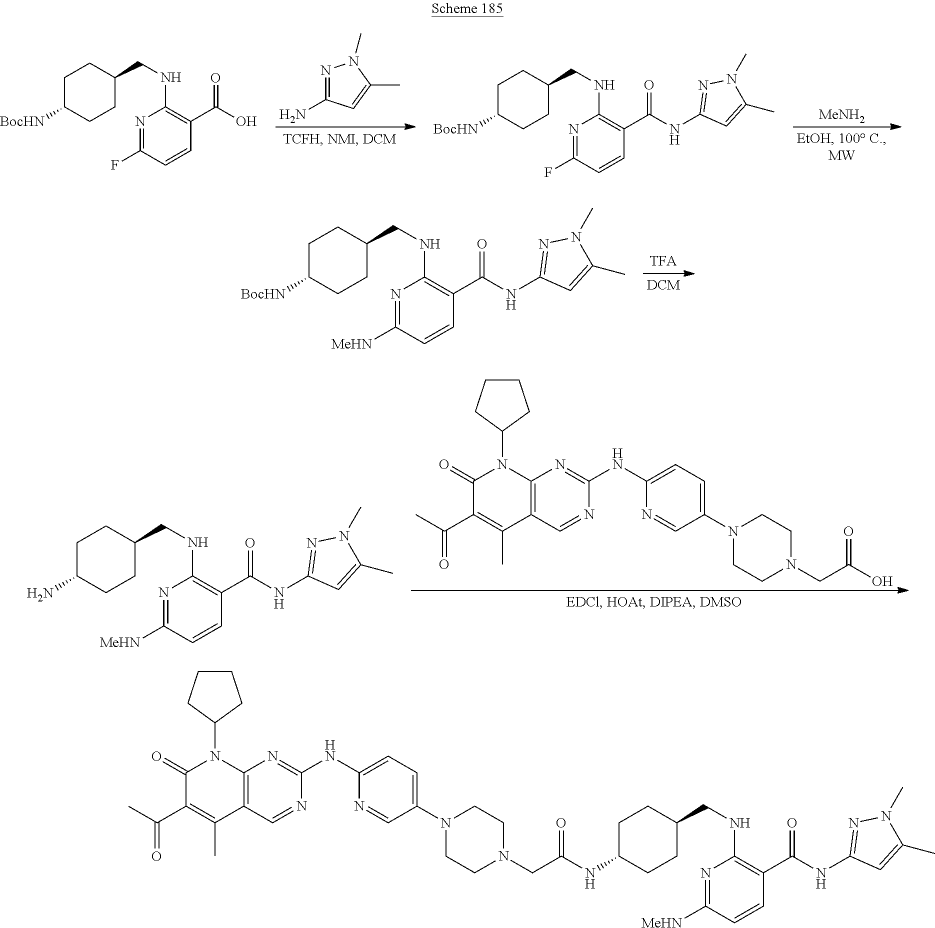

CPD-131 was synthesized following the procedures for preparing CPD-31 (8.2 mg, 16% yield over 4 steps) as a yellow solid. MS (ESI) m/z=859.8 [M+H]⁺.

### Example 186. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyano-6-cyclopropylpyridin-2-yl)-6-(methylamino)nicotinamide (CPD-132)

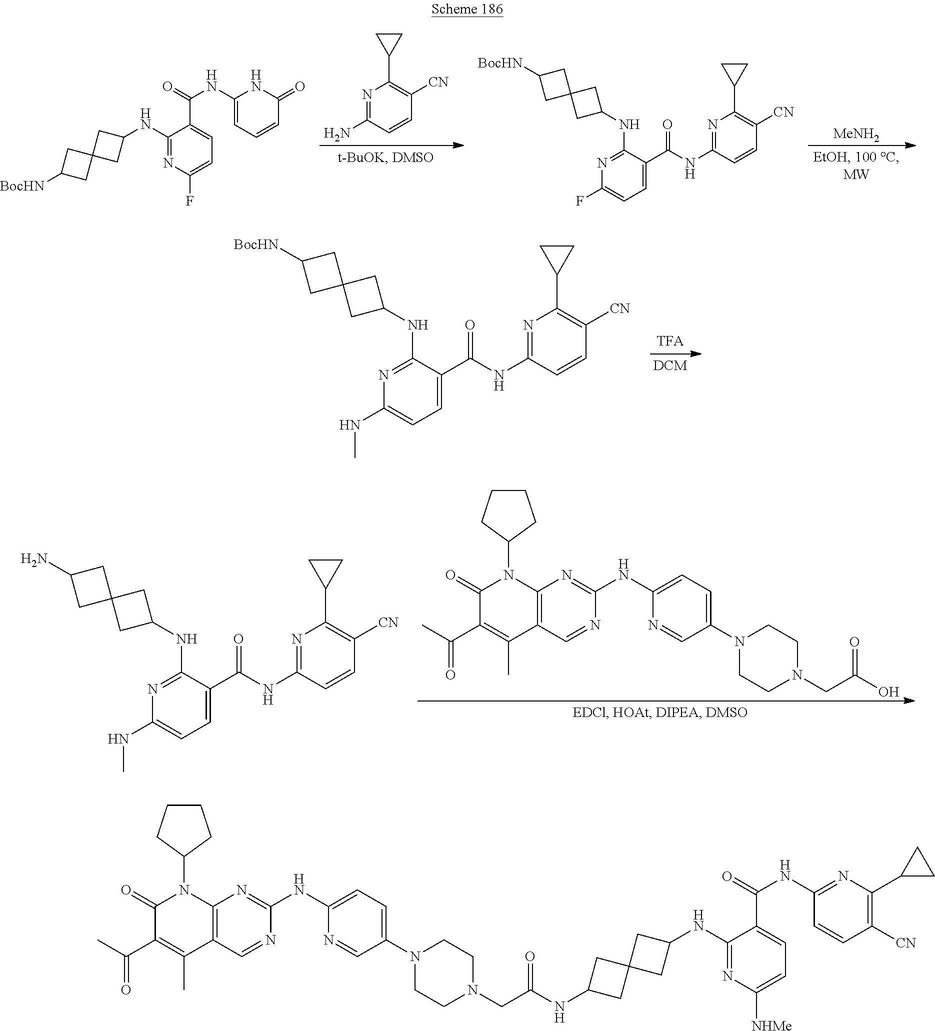

CPD-132 was synthesized following the procedures for preparing CPD-86 and CPD-25 (6 mg, 10% yield over 4 steps) as a yellow solid. MS (ESI) m/z=905.7 [M+H]⁺.

### Example 187. 2-((2-(4-(6-((6-Acetyl-8-cyclopentyl-5-methyl-7-oxo-7,8-dihydropyrido[2,3-d]pyrimidin-2-yl)amino)pyridin-3-yl)piperazine-1-carbonyl)-2-azaspiro[3.3]heptan-6-yl)amino)-N-(6-(dimethylamino)pyridazin-3-yl)-6-(methylamino)nicotinamide (CPD-133)

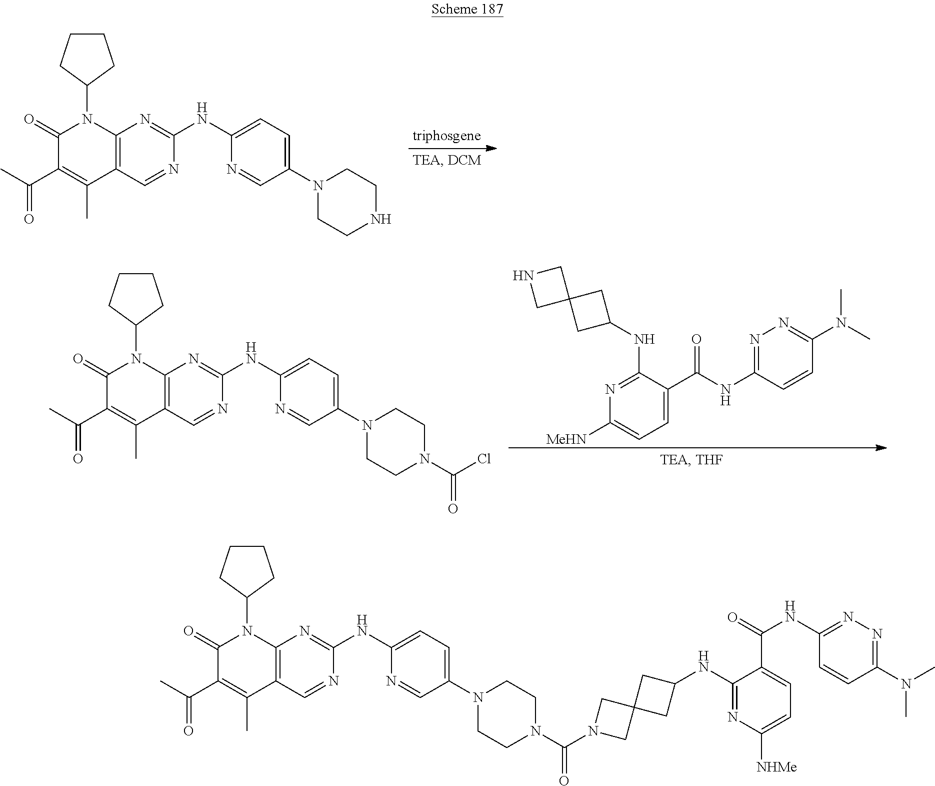 [SMILES: CC(=O)c1c(C)c2cnc(Nc3ccc(N4CCNCC4)cn3)nc2n(C2CCCC2)c1=O ; CC(=O)c1c(C)c2cnc(Nc3ccc(N4CCN(C(=O)Cl)CC4)cn3)nc2n(C2CCCC2)c1=O ; CNc1ccc(C(=O)Nc2ccc(N(C)C)nn2)c(NC2CC3(CNC3)C2)n1 ; CNc1ccc(C(=O)Nc2ccc(N(C)C)nn2)c(NC2CC3(C2)CN(C(=O)N2CCN(c4ccc(Nc5ncc6c(C)c(C(C)=O)c(=O)n(C7CCCC7)c6n5)nc4)CC2)C3)n1]

### Step 1. Synthesis of 4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl chloride

To a solution of 6-acetyl-8-cyclopentyl-5-methyl-2-((5-(piperazin-1-yl)pyridin-2-yl)amino)pyrido[2,3-d]pyrimidin-7(8H)-one (20 mg, 0.045 mmol) in DCM (5 mL) were added TEA (45.2 mg, 0.45 mmol) and triphosgene (13.3 mg, 0.045 mmol) at 0° C. The reaction mixture was stirred at 0° C. for 2 h, then it was quenched with H₂O (20 mL) and extracted with DCM (20 mL×3). The organic phase was washed with brine, dried over anhydrous Na₂SO₄, filtered, and concentrated. The residue was purified by reverse-phase chromatography to provide the desired product (13 mg, 57% yield) as a yellow oil. MS (ESI) m/z=510.5 [M+H]⁺.

### Step 2. Synthesis of 2-((2-(4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)-2-azaspiro[3.3]heptan-6-yl)amino)-N-(6-(dimethylamino)pyridazin-3-yl)-6-(methylamino)nicotinamide

A solution of 4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl chloride (13.4 mg, 0.03 mmol), 2-((2-azaspiro[3.3]heptan-6-yl)amino)-N-(6-(dimethylamino)pyridazin-3-yl)-6-(methylamino)nicotinamide (11.5 mg, 0.03 mmol) and TEA (30.3 mg, 0.3 mmol) in THF (5 mL) was stirred at rt for 2 h. The mixture was quenched with H₂O (20 mL) and extracted with ethyl acetate (20 mL×3). The organic phase was washed with brine, dried over anhydrous Na₂SO₄, filtered, and concentrated. The residue was purified by reverse-phase chromatography to provide the desired product (4.0 mg, 29% yield) as a yellow solid. MS (ESI) m/z=856.8 [M+H]⁺.

### Example 188. 4-(6-((6-Acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(methylamino)pyridin-2-yl)amino)spiro[3.3]heptan-2-yl)piperazine-1-carboxamide (CPD-134)

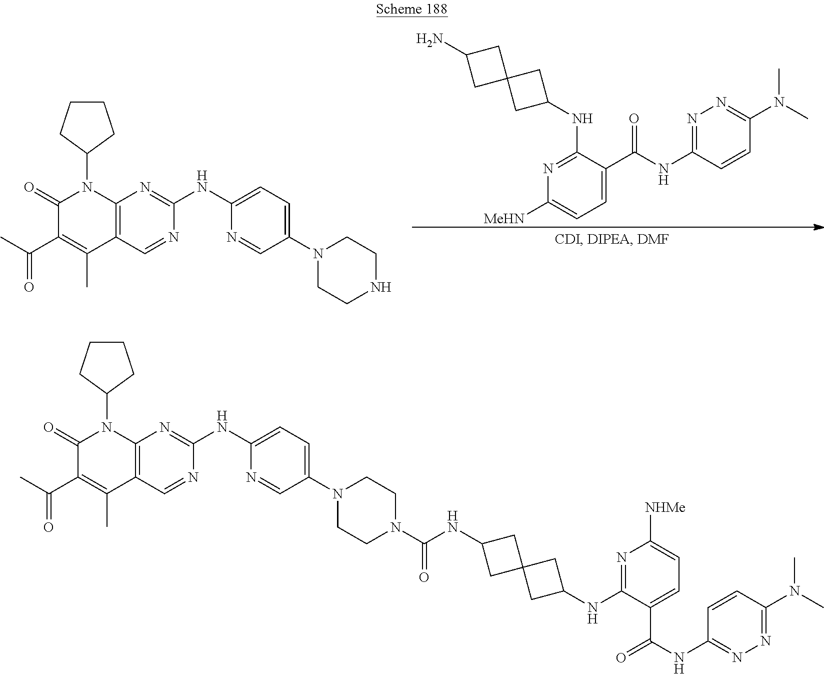 [SMILES: CC(=O)c1c(C)c2cnc(Nc3ccc(N4CCNCC4)cn3)nc2n(C2CCCC2)c1=O ; CNc1ccc(C(=O)Nc2ccc(N(C)C)nn2)c(NC2CC3(CC(N)C3)C2)n1 ; CNc1ccc(C(=O)Nc2ccc(N(C)C)nn2)c(NC2CC3(CC(NC(=O)N4CCN(c5ccc(Nc6ncc7c(C)c(C(C)=O)c(=O)n(C8CCCC8)c7n6)nc5)CC4)C3)C2)n1]

A mixture of 2-((6-aminospiro[3.3]heptan-2-yl)amino)-N-(6-(dimethylamino)pyridazin-3-yl)-6-(methylamino)nicotinamide (10 mg, 0.02 mmol), CDI (5.6 mg, 0.04 mmol) and DIPEA (5 mg, 0.04 mmol) in DMF (2 mL) was stirred at rt for 30 min. Then 6-acetyl-8-cyclopentyl-5-methyl-2-((5-(piperazin-1-yl)pyridin-2-yl)amino)pyrido[2,3-d]pyrimidin-7(8H)-one (17 mg, 0.4 mmol) was added. The reaction mixture was stirred at rt for additional 16 h. The reaction mixture was diluted with water (20 mL) and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography and prep-TLC to provide the desired product (7.3 mg, 38% yield) as a yellow solid. MS (ESI) m/z=870.8 [M+H]⁺.

### Example 189. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(methylamino)nicotinamide (CPD-135)

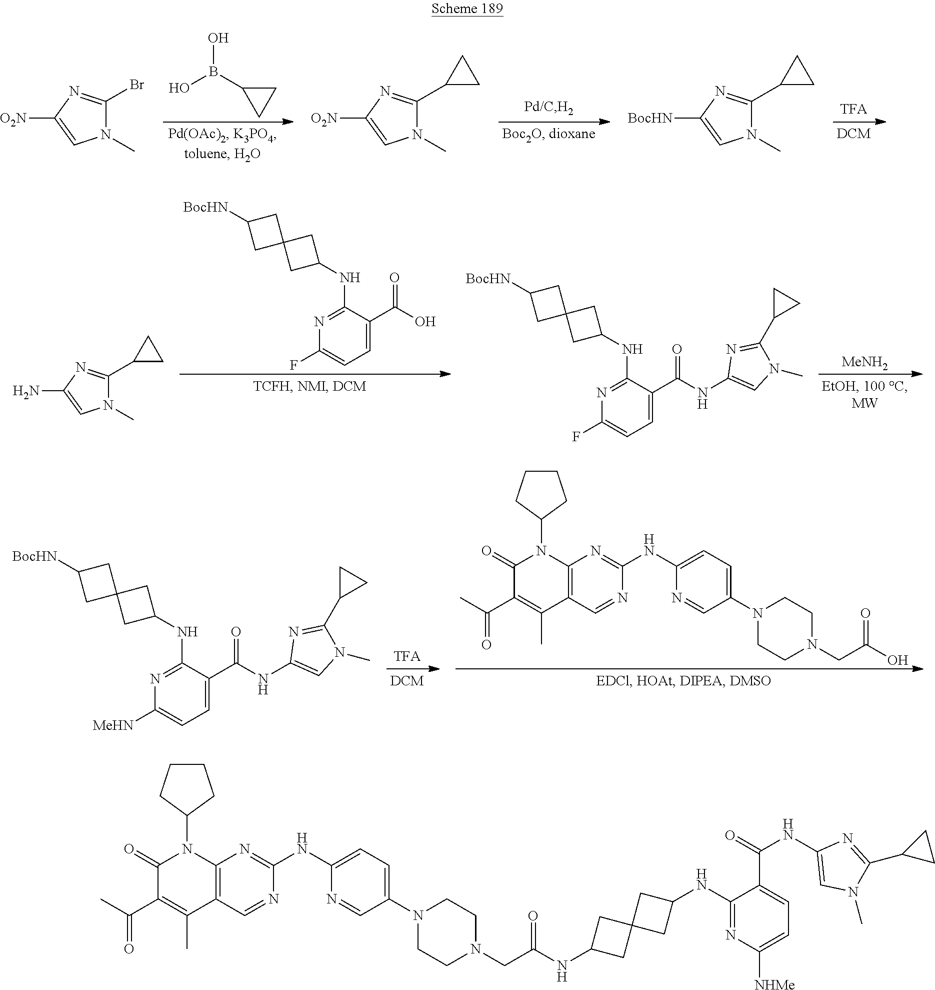

### Step 1. Synthesis of 2-cyclopropyl-1-methyl-4-nitro-1H-imidazole

To a mixture of 2-bromo-1-methyl-4-nitro-1H-imidazole (260 mg, 1.26 mmol) and cyclopropylboronic acid (542 mg, 6.31 mmol) in toluene (10 mL) and H₂O (1 mL) were added K₃PO₄ (803 mg, 3.79 mmol), S-phos (52 mg, 0. 12 mmol) and Pd(OAc)₂ (28 mg, 0.12 mmol) at rt under N₂. The reaction mixture was stirred at 100 [symbol] for 16 h. After cooling down to rt, the reaction mixture was diluted with water (20 mL) and extracted with ethyl acetate (3×20 mL). The combined organic layers were washed with brine, dried over anhydrous sodium sulfate, filtered, and concentrated. The residue was purified by reverse-phase chromatography to provide the desired product (190 mg, 90% yield) as a yellow solid. MS (ESI) m/z=168.2 [M+H]⁺.

### Step 2. Synthesis of tert-butyl (2-cyclopropyl-1-methyl-1H-imidazol-4-yl)carbamate

To a solution of 2-cyclopropyl-1-methyl-4-nitro-1H-imidazole (81 mg, 0.48 mmol) in dioxane (10 mL) were added Boc₂O (528 mg, 2.42 mmol), DIPEA (187 mg, 1.45 mmol) and 10% Pd/C (30 mg). The reaction mixture was stirred at 50° C. for 16 h under hydrogen balloon. Then the reaction mixture was filtered and concentrated. The resulting residue was purified by silica gel flash chromatography to provide the desired product (82 mg, 71% yield) as a white solid. MS (ESI) m/z=238.6 [M+H]⁺.

### Step 3. Synthesis of 2-cyclopropyl-1-methyl-1H-imidazol-4-amine

A solution of tert-butyl (2-cyclopropyl-1-methyl-1H-imidazol-4-yl)carbamate (17 mg, 0.07 mmol) in DCM (1 mL) and TFA (1 mL) was stirred at rt for 1 h. The solution was concentrated under vacuum to provide the crude product. MS (ESI) m/z=138.2 [M+H]⁺.

The remaining steps were performed according to the procedures for preparing CPD-31 to provide the desired product (12.8 mg, 19% yield over 4 steps) as a yellow solid. MS (ESI) m/z=883.8 [M+H]⁺.

### Example 190. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclopentyl)amino)-N-(6-(dimethylamino)pyridazin-3-yl)-6-(methylamino)nicotinamide (CPD-136)

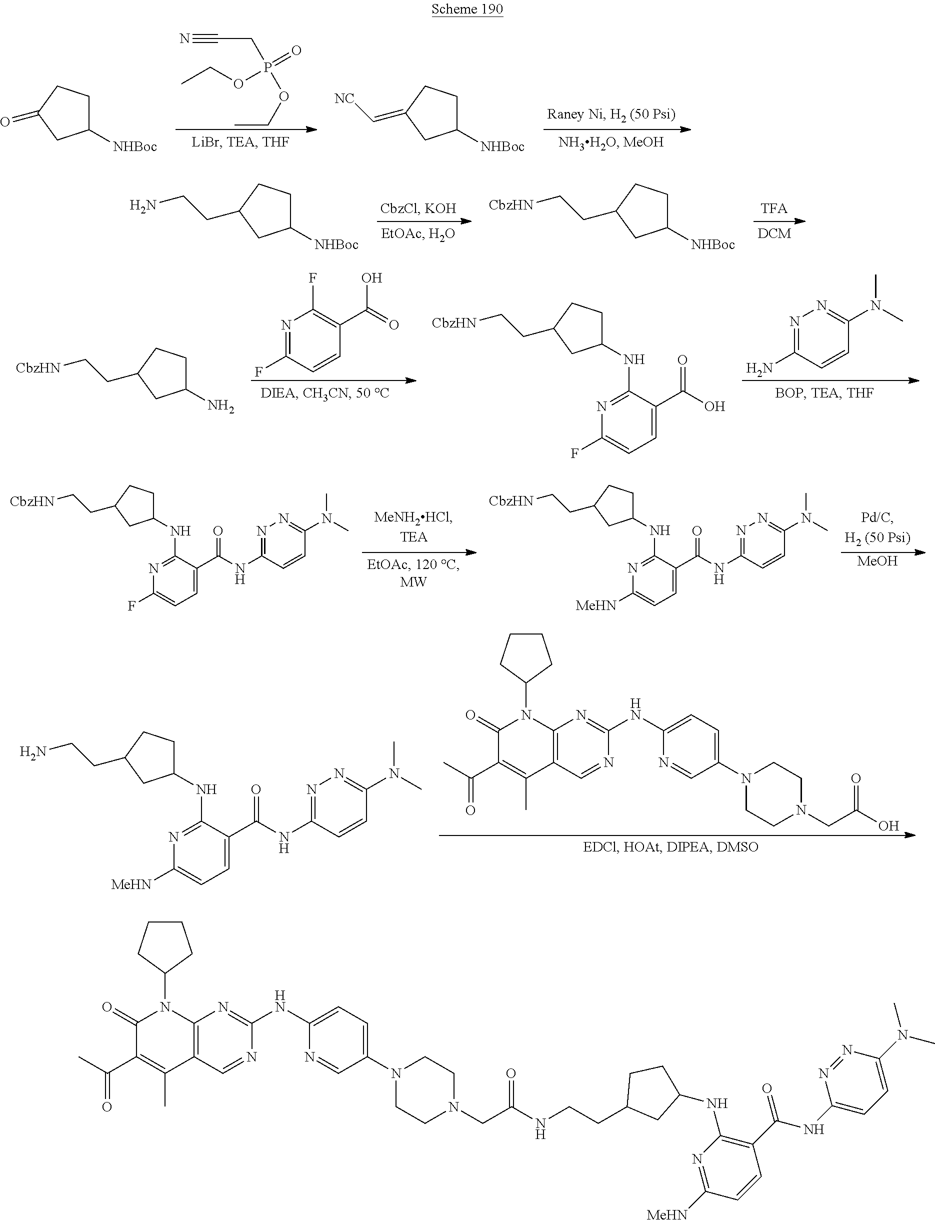

### Step 1. Synthesis of tert-butyl N-[(3E)-3-(cyanomethylene)cyclopentyl]carbamate

To a mixture of LiBr (840 mg, 9.67 mmol) and TEA (2.11 g, 20.8 mmol) in THF (10 mL) was added 2- [symbol] diethoxyphosphorylacetonitrile (1.86 g, 10.5 mmol) dropwise at 0. The mixture was stirring at rt for 0.5 h. Then tert-butyl N-(3-oxocyclopentyl)carbamate (0.90 g, 4.52 mmol) in THF (10 mL) was dropwise to the mixture at 0 [symbol] . After the mixture was stirred at rt for additional 12 h, it was poured into water (50 mL) and extracted with DCM (50 mL×3). The combined organic phase was washed with brine (50 mL×4), dried with anhydrous Na₂SO₄, filtered, and concentrated in vacuum. The residue was purified by silica gel column chromatography (petroleum ether/ethyl acetate=15:1 to 10:1) to provide the desired product (730 mg, 73% yield) as a yellow oil. ¹HNMR (400 MHz, CDCl₃) δ 5.29-5.23 (m, 1H), 4.63-4.39 (m, 1H), 4.21-3.97 (m, 1H), 3.11-2.98 (m, 1H), 2.94-2.73 (m, 1H), 2.69-2.57 (m, 1H), 2.56-2.35 (m, 2H), 2.20-2.08 (m, 1H), 1.79-1.65 (m, 1H), 1.46 (s, 9H). MS (ESI) m/z=167.3 [M+H-56]⁺.

### Step 2. Synthesis of tert-butyl N-[3-(2-aminoethyl)cyclopentyl]carbamate

To a mixture of tert-butyl N-[(3E)-3-(cyanomethylene)cyclopentyl]carbamate (0.70 g, 3.15 mmol) in MeOH (7 mL) was added NH₃·H₂O (6.37 g, 54.5 mmol) and Raney-Ni (140 mg, 163 μmol) at rt. The reaction mixture was stirred at rt for 12 h under H₂ (50 psi). The reaction mixture was filtered and the filtrate was concentrated to provide the desired product (0.71 g, 98% yield) as a yellow oil. ¹HNMR (400 MHz, CDCl₃) b 4.58-4.53 (m, 1H), 4.08-3.82 (m, 1H), 2.27 (s, 1H), 2.14-1.55 (m, 5H), 1.44 (s, 9H), 1.39-1.32 (m, 1H), 1.30-1.16 (m, 1H), 1.12-0.92 (m, 1H).

### Step 3. Synthesis of tert-butyl N-[3-[2-(benzyloxycarbonylamino)ethyl]cyclopentyl]carbamate

To a mixture of tert-butyl N-[3-(2-aminoethyl)cyclopentyl]carbamate (0.70 g, 3.07 mmol) in EtOAc (10 mL) was added KOH (350 mg, 6.24 mmol) in H₂O (10 mL) at rt. Then CbzCl (693 mg, 4.06 mmol) was added dropwise at rt. After the reaction mixture was stirred at rt for 1 h, it was diluted with water (10 mL) and extracted with ethyl acetate (30 mL×2). The combined organic phase was washed with brine (30 mL), dried with anhydrous Na₂SO₄, filtered, and concentrated in vacuum to provide the crude product (0.91 g, 82% yield) as a light-yellow oil. MS (ESI) m/z=263.3 [M+H-100]⁺.

### Step 4. Synthesis of benzyl N-[2-(3-aminocyclopentyl)ethyl]carbamate

To a mixture of tert-butyl N-[3-[2-(benzyloxycarbonylamino)ethyl]cyclopentyl]carbamate (0.90 g, 2.48 mmol) in DCM (10 mL) was added TFA (2 mL) at rt. The reaction mixture was stirred at rt for 12 h. The reaction mixture was concentrated in vacuum to provide the desired product (1.00 g, crude) as a brown oil. MS (ESI) m/z=263.4 [M+H]⁺.

### Step 5. Synthesis of 2-[[3-[2-(benzyloxycarbonylamino)ethyl]cyclopentyl]amino]-6-fluoro-pyridine-3-carboxylic acid

To a mixture of benzyl N-[2-(3-aminocyclopentyl)ethyl]carbamate (0.90 g, 2.39 mmol,) and 2,6-difluoropyridine-3-carboxylic acid (900 mg, 5.66 mmol) in CH₃CN (9 mL) was added DIEA (2.23 g, 17.2 mmol) at 20° C. The reaction mixture was stirred at 50° C. for 12 h. After cooling down to rt, the mixture was diluted with water (50 mL) and extracted with ethyl acetate (50 mL×3). The combined organic phase was washed with brine (50 mL×2), dried with anhydrous Na₂SO₄, filtered, and concentrated in vacuum to provide the desired product (1.00 g, crude) as a brown solid. MS (ESI) m/z=402.2 [M+H]⁺.

### Step 6. Synthesis of benzyl N-[2-[3-[[3-[[6-(dimethylamino)pyridazin-3-yl]carbamoyl]-6-fluoro-2-pyridyl]amino]cyclopentyl]ethyl]carbamate

To a mixture of 2-[[3-[2-(benzyloxycarbonylamino)ethyl]cyclopentyl]amino]-6-fluoro-pyridine-3-carboxylic acid (0.99 g, 2.47 mmol) and N³,N³-dimethylpyridazine-3,6-diamine (0.40 g, 2.89 mmol) in THF (10 mL) were added BOP (1.50 g, 3.39 mmol) and TEA (1.45 g, 14.4 mmol) at rt. The reaction mixture was stirred at rt for 12 h. The mixture was diluted with water (50 mL) and extracted with ethyl acetate (100 mL×3). The combined organic phase was washed with brine (100 mL), dried with anhydrous Na₂SO₄, filtered, and concentrated in vacuum. The residue was purified by prep-HPLC to provide the desired product (0.50 g, 39% yield) as a yellow oil. MS (ESI) m/z=522.4 [M+H]⁺.

### Step 7. Synthesis of benzyl N-[2-[3-[[3-[[6-(dimethylamino)pyridazin-3-yl]carbamoyl]-6-(methylamino)-2-pyridyl]amino]cyclopentyl]ethyl]carbamate

To a solution of benzyl N-[2-[3-[[3-[[6-(dimethylamino)pyridazin-3-yl]carbamoyl]-6-fluoro-2-pyridyl]amino]cyclopentyl]ethyl]carbamate (0.50 g, 959 μmol) in EtOH (5 mL) were added methanamine hydrochloride (600 mg, 8.89 mmol) and TEA (1.02 g, 10.1 mmol) at rt. The reaction [symbol] mixture was stirred at 120° C. for 2 h under microwave irradiation. After cooling down to rt, the residue was diluted with water (20 mL) and extracted with ethyl acetate (20 mL×3). The combined organic phase was washed with brine (50 mL), dried with anhydrous Na₂SO₄, filtered, and concentrated in vacuum to provide the desired product (0.40 g, 78% yield) as a brown solid. ¹HNMR (400 MHz, CDCl₃) δ 8.56 (s, 1H), 8.15 (d, J=9.6 Hz, 1H), 7.61 (d, J=8.8 Hz, 1H), 7.41-7.29 (m, 5H), 6.89 (d, J=10.0 Hz, 1H), 5.68 (d, J=8.4 Hz, 1H), 5.10 (s, 2H), 4.75 (s, 2H), 4.55-4.34 (m, 1H), 3.23-3.19 (m, 2H), 3.14 (s, 6H), 2.96 (d, J=4.8 Hz, 3H), 2.46-2.27 (m, 1H), 2.25-2.10 (m, 1H), 1.95-1.82 (m, 2H), 1.65-1.49 (m, 4H), 1.43-1.32 (m, 1H), 1.20-1.05 (m, 1H). MS (ESI) m/z=533.3 [M+H]⁺.

### Step 8. Synthesis of 2-[[3-(2-aminoethyl)cyclopentyl]amino]-N-[6-(dimethylamino)pyridazin-3-yl]-6-(methylamino)pyridine-3-carboxamide

To a mixture of benzyl N-[2-[3-[[3-[[6-(dimethylamino)pyridazin-3-yl]carbamoyl]-6-(methylamino)-2-pyridyl]amino]cyclopentyl]ethyl]carbamate (120 mg, 225 mol) in THF (2 mL) was added Pd/C (12.0 mg, 93.9 μmol) at rt. The reaction mixture was stirred at rt for 12 h under H₂ (50 psi). The reaction mixture was filtered. The filtrate was concentrated and purified by prep-HPLC to provide the desired product (53.5 mg, 59% yield) as a yellow solid. ¹HNMR (400 MHz, DMSO-d₆) δ 10.48 (s, 1H), 8.68 (s, 1H), 8.06 (d, J=10.0 Hz, 1H), 7.88 (d, J=7.6 Hz, 1H), 7.74-7.66 (m, 3H), 7.43-6.89 (m, 1H), 5.75 (d, J=8.8 Hz, 1H), 4.44-4.33 (m, 1H), 3.21 (s, 6H), 2.85-2.76 (m, 5H), 2.36-2.28 (m, 1H), 2.08-1.79 (m, 3H), 1.63-1.56 (m, 2H), 1.50-1.41 (m, 1H), 1.33-1.13 (m, 1.5H), 1.06-0.97 (s, 1H). MS (ESI) m/z=399.5 [M+H]⁺.

### Step 9. Synthesis of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclopentyl)amino)-N-(6-(dimethylamino)pyridazin-3-yl)-6-(methylamino)nicotinamide

A mixture of 2-((3-(2-aminoethyl)cyclopentyl)amino)-N-(6-(dimethylamino)pyridazin-3-yl)-6-(methylamino)nicotinamide (10 mg, 0.02 mmol), 5-((3-((6-(dimethylamino)pyridazin-3-yl)carbamoyl)-6-(methylamino)pyridin-2-yl)amino)pentanoic acid (10 mg, 0.02 mmol), EDCl (5.6 mg, 0.03 mmol), HOAt (3.9 mg, 0.03 mmol) and DIPEA (12.6 mg, 0.1 mmol) in DMSO (2 mL) was stirred at rt for 16 h. The reaction mixture was diluted with water (20 mL) and extracted with ethyl acetate (3×20 mL). The combined organic layers were washed with saturated brine (50 mL), dried over anhydrous sodium sulfate, filtered, and evaporated under reduced pressure. The resulting residue was purified by reverse-phase chromatography and pre-TLC to provide the desired product (7.9 mg, 41% yield) as a light yellow solid. MS (ESI) m/z=886.7 [M+H]⁺.

### Example 191. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(pyridin-2-yl)nicotinamide (CPD-137)

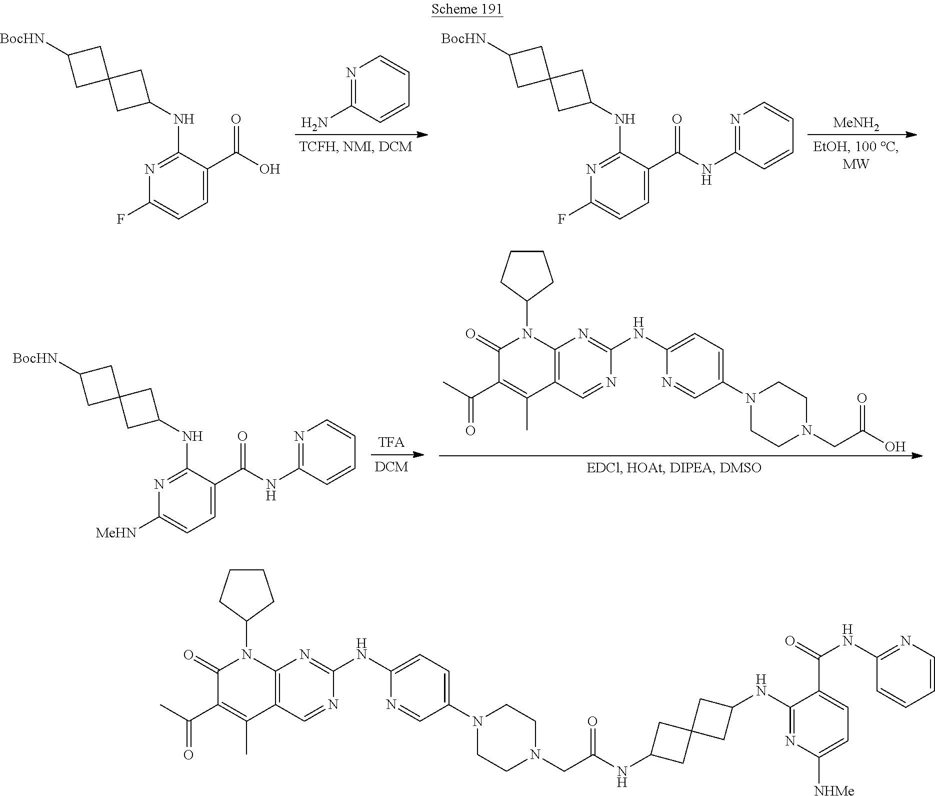

CPD-137 was synthesized following the procedures for preparing CPD-31 (15.6 mg, 26% yield over 4 steps) as a yellow solid. MS (ESI) m/z=840.7 [M+H]⁺.

### Example 192. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-cyclopropyl-N-(6-cyclopropylpyridin-2-yl)nicotinamide (CPD-138)

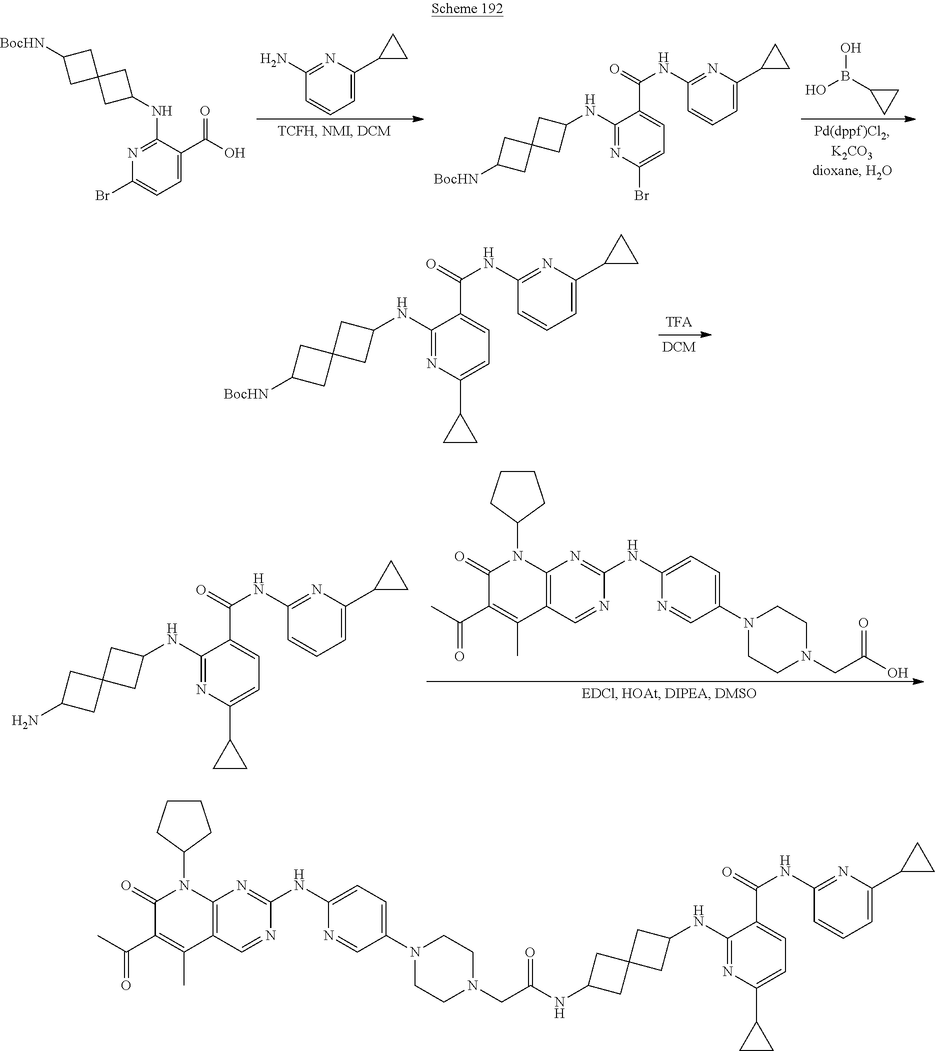

CPD-138 was synthesized following the procedures for preparing CPD-44 (6.0 mg, 10% yield over 4 steps) as a yellow solid. MS (ESI) m/z=891.7 [M+H]⁺.

### Example 193. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-(isopropylamino)nicotinamide (CPD-139)

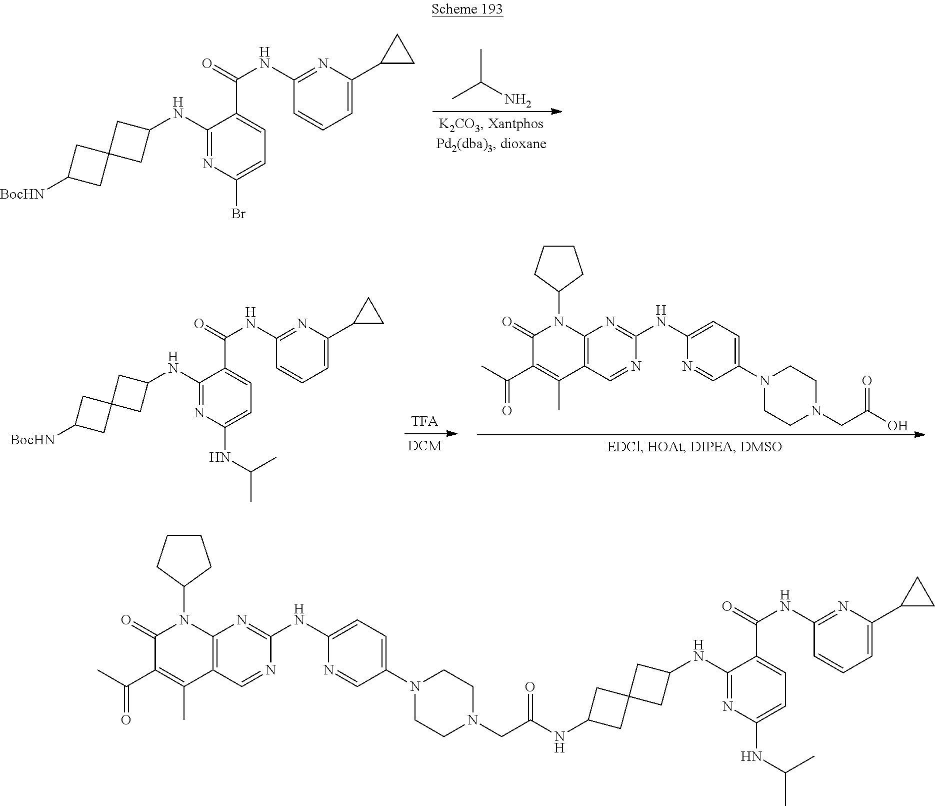

### Step 1. Synthesis of tert-butyl (6-((3-((6-cyclopropylpyridin-2-yl)carbamoyl)-6-(isopropylamino)pyridin-2-yl)amino)spiro[3.3]heptan-2-yl)carbamate

To a solution of tert-butyl (6-((6-bromo-3-((6-cyclopropylpyridin-2-yl)carbamoyl)pyridin-2-yl)amino)spiro[3.3]heptan-2-yl)carbamate (30 mg, 0.055 mmol) in dioxane (6 mL) were added propan-2-amine (33 mg, 0.55 mmol), Pd₂(dba)₃ (5 mg, 5.5 μmol), Xantphos (10 mg, 0.016 mmol) and K₂CO₃ (38 mg, 0.28 mmol) at rt. The reaction mixture was stirred at 90° C. for 6 h under argon atmosphere. After cooling down to rt, the reaction mixture was diluted with water (30 mL) and extracted with ethyl acetate (3×30 mL). The combined organic layers were washed with saturated brine (30 mL), dried over anhydrous sodium sulfate, filtered, and evaporated under reduced pressure. The resulting residue was purified by silica gel chromatography to provide the desired product (22 mg, 76% yield) as a light-yellow solid. MS (ESI) m/z=521.7 [M+H]⁺.

The remaining steps were performed according to the procedures for preparing CPD-25 to provide the desired product (9 mg, 24% yield over 2 steps) as a yellow solid. MS (ESI) m/z=908.7 [M+H]⁺.

### Example 194. 2-((6-(4-(6-((6-Acetyl-8-cyclopentyl-5-methyl-7-oxo-7,8-dihydropyrido[2,3-d]pyrimidin-2-yl)amino)pyridin-3-yl)piperazine-1-carbonyl)spiro[3.3]heptan-2-yl)amino)-N-(6-(dimethylamino)pyridazin-3-yl)-6-(methylamino)nicotinamide (CPD-140)

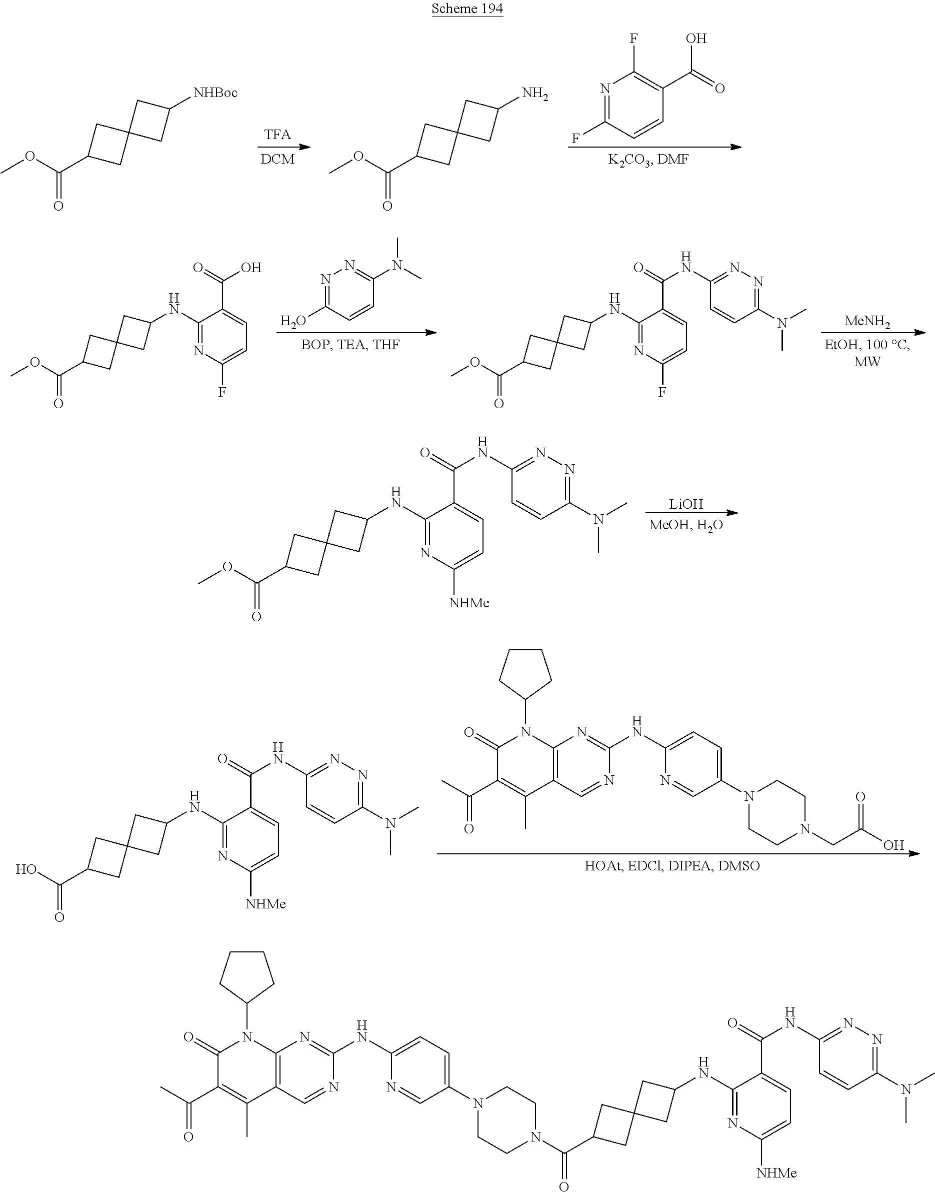

CPD-140 was synthesized following the procedures for preparing CPD-82 (5.0 mg, 3% yield over 6 steps) as a yellow solid. MS (ESI) m/z=855.8 [M+H]⁺.

### Example 195. 2-((6-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-cyano-N-(6-cyclopropylpyridin-2-yl)nicotinamide (CPD-141)

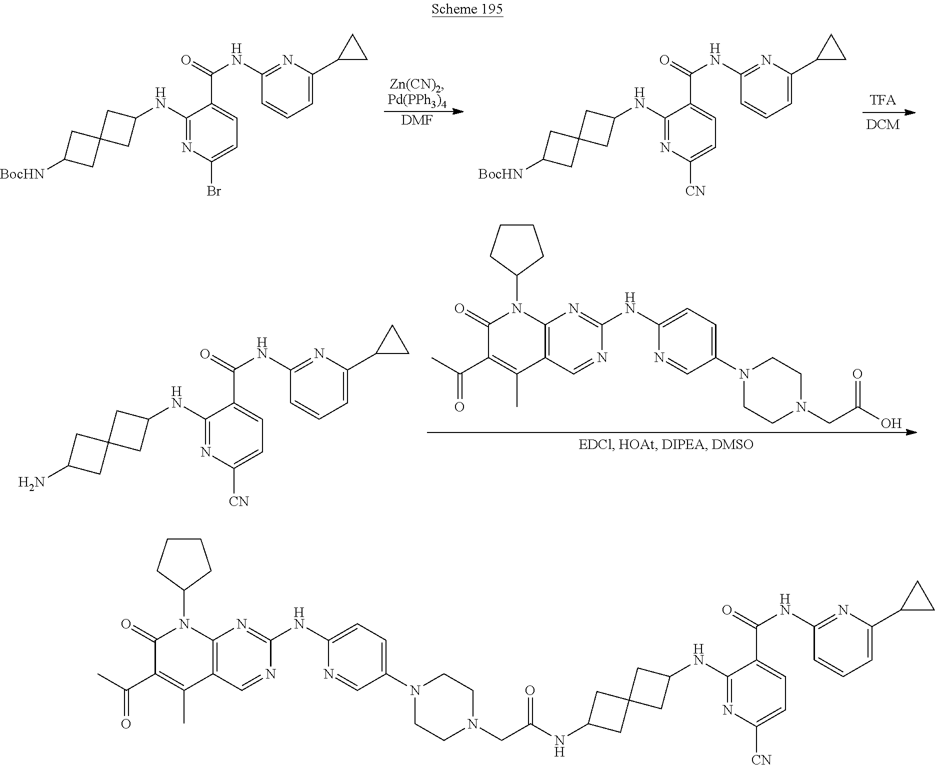

### Step 1. Synthesis of tert-butyl (6-((6-cyano-3-((6-cyclopropylpyridin-2-yl)carbamoyl)pyridin-2-yl)amino)spiro[3.3]heptan-2-yl)carbamate

To a solution of tert-butyl (6-((6-bromo-3-((6-cyclopropylpyridin-2-yl)carbamoyl)pyridin-2-yl)amino)spiro[3.3]heptan-2-yl)carbamate (50 mg, 92.17 mmol) in DMF (5 mL) were added Zn(CN)₂ (22 mg, 0.18 mmol) and Pd(PPh₃)₄ (21 mg, 0.018 mmol) at rt. The reaction mixture was stirred at 90° C. for 3 h under argon atmosphere. After cooling down to rt, the reaction mixture was diluted with water (30 mL) and extracted with ethyl acetate (3×30 mL). The combined organic layers were washed with saturated brine (30 mL), dried over anhydrous sodium sulfate, filtered, and evaporated under reduced pressure. The resulting residue was purified by silica gel chromatography to provide the desired product (30 mg, 67% yield) as a white solid. MS (ESI) m/z=489.7 [M+H]⁺.

The remaining steps were performed according to the procedures for preparing CPD-25 to provide the desired product (15 mg, 28% yield over 2 steps) as a yellow solid. MS (ESI) n/z=876.8 [M+H]⁺.

### Example 196. 6-((7-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-6′-cyano-N-(4,5-dimethylthiazol-2-yl)-[2,3′-b]pyridine]-5-carboxamide (CPD-142)

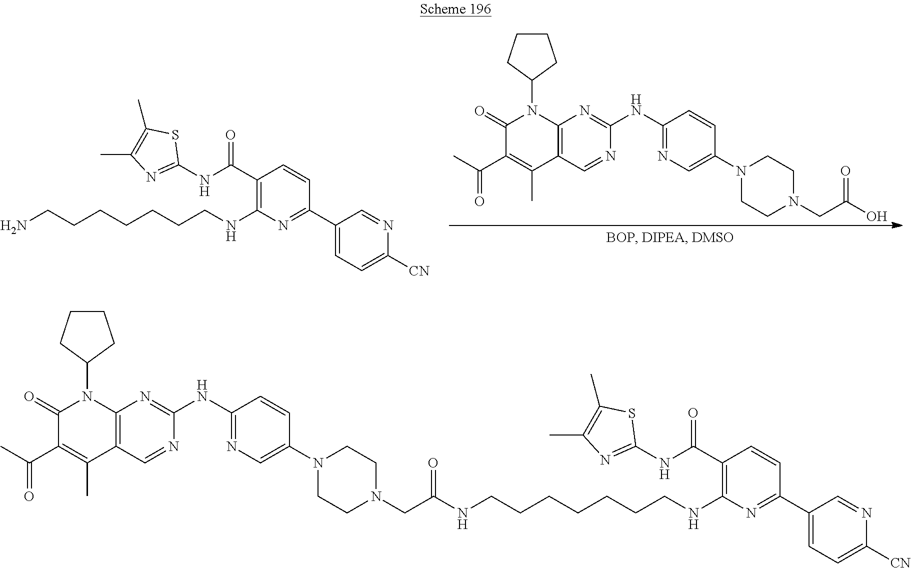 [SMILES: Cc1nc(NC(=O)c2ccc(-c3ccc(C#N)nc3)nc2NCCCCCCCN)sc1C ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCCCCCCCNc5nc(-c6ccc(C#N)nc6)ccc5C(=O)Nc5nc(C)c(C)s5)CC4)cn3)nc2n(C2CCCC2)c1=O]

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (16.83 mg, 33.29 mmol) in DMSO (3 mL) were added DIPEA (21.51 mg, 166.47 mmol, 27.51 mL), BOP (10.19 mg, 49.94 mmol) and 2-(7-aminoheptylamino)-6-(6-cyano-3-pyridyl)-N-(4,5-dimethylthiazol-2-yl)pyridine-3-carboxamide (TFA salt, 25 mg, 43.28 mmol). The mixture was stirred at rt for 2 h, before it was diluted with EtOAc (20 mL) and washed with water (15 mL). The aqueous phase was extracted with EtOAc (15 mL×3). The combined organic phase was concentrated under reduced pressure and purified by prep-TLC (DCM/MeOH=20:1) and prep-HPLC to provide the title compound (15.5 mg, 48% yield) as a yellow solid. MS (ESI) m/z=476.46 [M/2+H]⁺.

### Example 197. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-4-chloro-N-(6-cyclopropyl-5-methylpyridin-2-yl)benzamide (CPD-255)

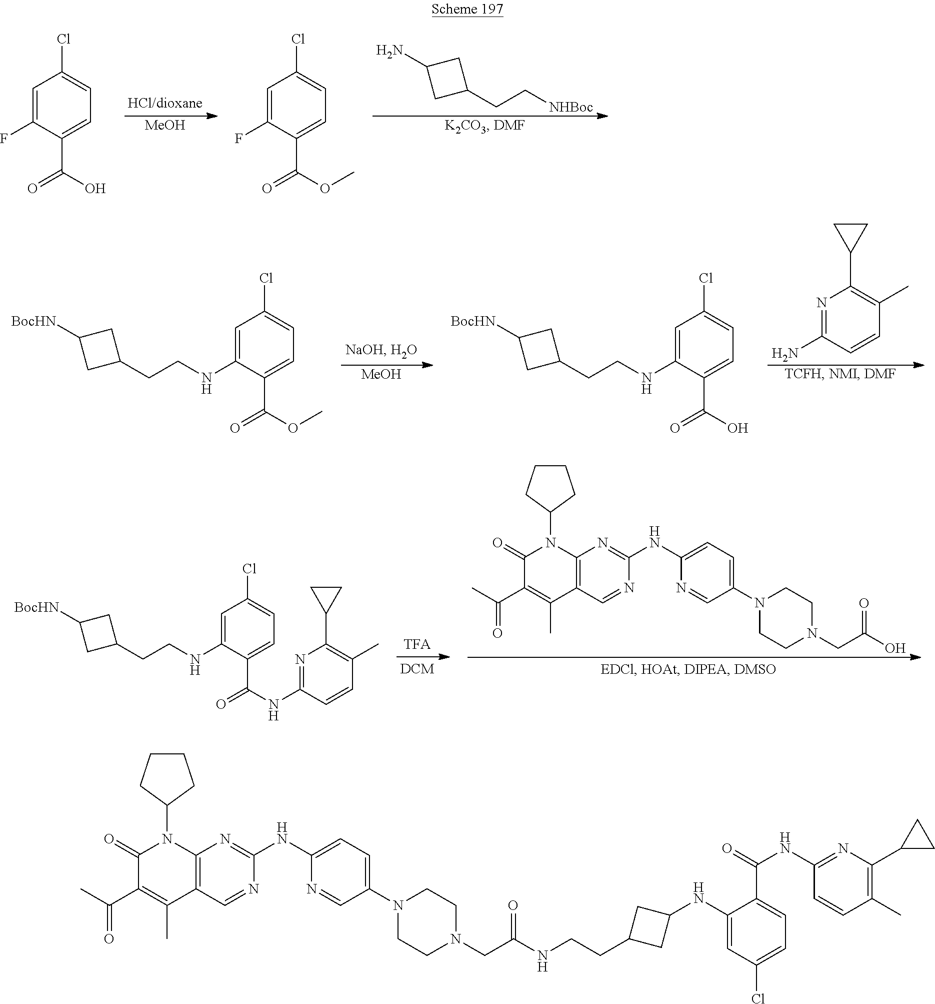

### Step 1. Synthesis of methyl 4-chloro-2-fluorobenzoate

To a solution of 4-chloro-2-fluoro-benzoic acid (2.0 g, 11.46 mmol) in MeOH (20 mL) was added HCl solution (4 M in dioxane, 10 mL, 40.0 mmol) at 0 [symbol] . The mixture was stirred at 80 [symbol] under inert atmosphere for 2 h. The reaction mixture was cooled to rt, diluted with water (100 mL), and extracted with ethyl acetate (3×50 mL). The combined organic layers were washed with saturated brine (100 mL), dried over anhydrous sodium sulfate, filtered, and evaporated under reduced pressure. The resulting residue was purified by silica gel chromatography to provide the desired product (2.0 g, 93% yield) as a pale-white solid. MS (ESI) m/z=189.5 [M+H]⁺.

### Step 2. Synthesis of methyl 2-((3-(2-((tert-butoxycarbonyl)amino)ethyl)cyclobutyl)amino)-4-chlorobenzoate

To a solution of methyl 4-chloro-2-fluoro-benzoate (80.0 mg, 0.42 mmol) in DMF (4 mL) were added methyl 4-chloro-2-fluoro-benzoate (80.0 mg, 0.42 mmol) and K₂CO₃ (117.1 mg, 0.85 mmol) at rt. The reaction mixture was stirred at 100 [symbol] for 16 h. The mixture was cooled to rt, diluted with water (50 mL), and extracted with ethyl acetate (3×20 mL). The combined organic layers were washed with saturated brine (50 mL), dried over anhydrous sodium sulfate, filtered, and evaporated under reduced pressure. The resulting residue was purified by silica gel chromatography to provide the desired product (45.0 mg, 28% yield) as a white solid. MS (ESI) m/z=383.9 [M+H]⁺.

### Step 3. Synthesis of 2-((3-(2-((tert-butoxycarbonyl)amino)ethyl)cyclobutyl)amino)-4-chlorobenzoic acid

To a solution of methyl 2-[[3-[2-(tert-butoxycarbonylamino)ethyl]cyclobutyl]amino]-4-chloro-benzoate (45.0 mg, 0.12 mmol) in MeOH (5 mL) and water (1 mL) was added NaOH (18.8 mg, 0.47 mmol) at rt. The reaction mixture was stirred at 80 [symbol] for 1 h. After cooling down to rt, the mixture was diluted with water (30 mL), acidified with HCl solution (1 M), and extracted with ethyl acetate (3×10 mL). The combined organic layers were washed with saturated brine (15 mL), dried over anhydrous sodium sulfate, filtered, and evaporated under reduced pressure. The resulting residue was purified by reverse-phase chromatography to provide the desired product (40.0 mg, 92% yield) as a white solid. MS (ESI) m/z=369.9 [M+H]⁺.

### Step 4. Synthesis of tert-butyl (2-(3-((5-chloro-2-((6-cyclopropyl-5-methylpyridin-2-yl)carbamoyl)phenyl)amino)cyclobutyl)ethyl)carbamate

To a solution of 2-[[3-[2-(tert-butoxycarbonylamino)ethyl]cyclobutyl]amino]-4-chloro-benzoic acid (40.0 mg, 0.11 mmol) in DMF (5 mL) were added 6-cyclopropyl-5-methyl-pyridin-2-amine (16.07 mg, 0.11 mmol), TCFH (45.6 mg, 0.16 mmol) and NMI (31.2 mg, 0.38 mmol). The reaction mixture was stirred at rt for 4 h. The reaction mixture was poured into water (30 mL) and extracted with ethyl acetate (3×20 mL). The combined organic layers were washed with saturated brine (15 mL), dried over anhydrous sodium sulfate, filtered, and evaporated under reduced pressure. The resulting residue was purified by reverse-phase chromatography to provide the desired product (32.0 mg, 59% yield) as a white solid. MS (ESI) m/z=500.1 [M+H]⁺.

### Step 5. Synthesis of 2-((3-(2-aminoethyl)cyclobutyl)amino)-4-chloro-N-(6-cyclopropyl-5-methylpyridin-2-yl)benzamide

A solution of tert-butyl N-[2-[3-[5-chloro-2-[(6-cyclopropyl-5-methyl-2-pyridyl)carbamoyl]anilino]cyclobutyl]ethyl]carbamate (32.0 mg, 64.1 μmol) in DCM (2 mL) and TFA (1 mL) was stirred at rt for 1 h. The solution was concentrated and purified by reverse-phase chromatography to provide the desired product (20.0 mg, 78% yield) as a light yellow solid. MS (ESI) m/z=399.9 [M+H]⁺.

### Step 6. Synthesis of 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-4-chloro-N-(6-cyclopropyl-5-methylpyridin-2-yl)benzamide

A mixture of 2-[4-[6-[(8-cyclopentyl-6-formyl-5-methyl-7-oxo-pyrido[2,3-d]pyrimidin-2-yl)amino]-3-pyridyl]piperazin-1-yl]acetic acid (12.3 mg, 25.1 μmol), 2-[[3-(2-aminoethyl)cyclobutyl]amino]-4-chloro-N-(6-cyclopropyl-5-methyl-2-pyridyl)benzamide (10.0 mg, 25.1 μmol), EDCl (7.2 mg, 37.6 μmol), HOAt (5.1 mg, 37.6 μmol) and DIPEA (16. 1 mg, 125 mol) in DMSO (2 mL) was stirred at rt for 16 h. The reaction mixture was poured into water (20 mL) and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography to provide the desired product (8.2 mg, 37% yield) as a light yellow solid. MS (ESI) m/z=886.8 [M+H]⁺.

### Example 198. 4-(6-((6-Acetyl-8-cyclopentyl-5-methyl-7-oxo-7,8-dihydropyrido[2,3-d]pyrimidin-2-yl)amino)pyridin-3-yl)-N-(2-(3-((5-chloro-2-((6-cyclopropyl-5-methylpyridin-2-yl)carbamoyl)phenyl)amino)cyclobutyl)ethyl)piperazine-1-carboxamide (CPD-256)

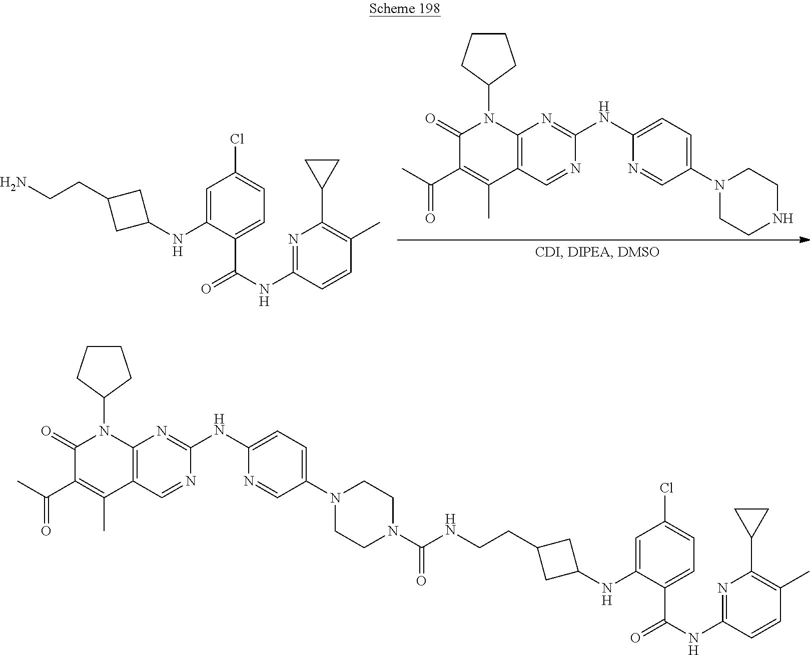 [SMILES: Cc1ccc(NC(=O)c2ccc(Cl)cc2NC2CC(CCN)C2)nc1C1CC1 ; CC(=O)c1c(C)c2cnc(Nc3ccc(N4CCNCC4)cn3)nc2n(C2CCCC2)c1=O ; CC(=O)c1c(C)c2cnc(Nc3ccc(N4CCN(C(=O)NCCC5CC(Nc6cc(Cl)ccc6C(=O)Nc6ccc(C)c(C7CC7)n6)C5)CC4)cn3)nc2n(C2CCCC2)c1=O]

To a solution of 2-((3-(2-aminoethyl)cyclobutyl)amino)-4-chloro-N-(6-cyclopropyl-5-methylpyridin-2-yl)benzamide (10.0 mg, 25.1 mol) in DMSO (2 mL) were added CDI (4.1 mg, 25.1 μmol) and DIPEA (6.5 mg, 50.1 mol). The reaction mixture was stirred at rt for 0.5 h. Then 6-acetyl-8-cyclopentyl-5-methyl-2-((5-(piperazin-1-yl)pyridin-2-yl)amino)pyrido[2,3-d]pyrimidin-7(8H)-one (11.2 mg, 25.1 μmol) was added and the mixture was stirred at rt for another 0.5 h. The reaction mixture was diluted with water (20 mL) and extracted with ethyl acetate (3×10 mL). The combined organic layers were washed with saturated brine (15 mL), dried over anhydrous sodium sulfate, filtered, and evaporated under reduced pressure. The resulting residue was purified by reverse-phase chromatography to provide the desired product (4.1 mg, 19% yield) as a light yellow solid. MS (ESI) m/z=872.7 [M+H]⁺.

### Example 199. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-4-chloro-N-(6-(dimethylamino)pyridazin-3-yl)benzamide (CPD-257)

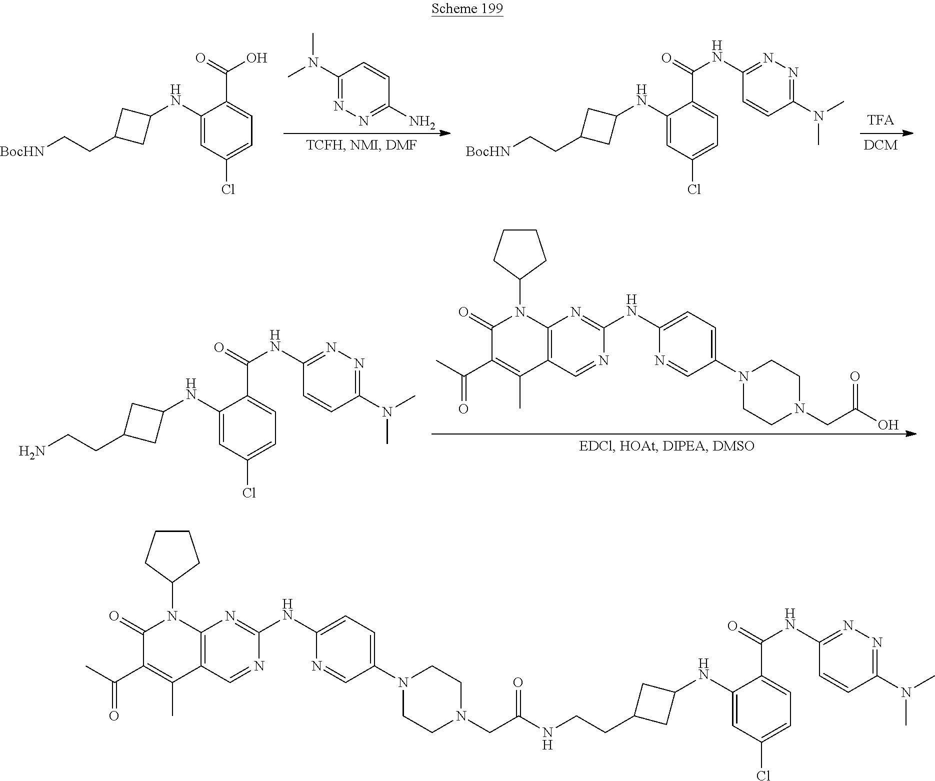

CPD-257 was synthesized following the procedures for preparing CPD-255 (4.0 mg, 4% yield over 3 steps) as a light-yellow solid. MS (ESI) m/z=876.8 [M+H]⁺.

### Example 200. 2-((3-(2-(2-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(6-(dimethylamino)pyridazin-3-yl)-4-fluorobenzamide (CPD-258)

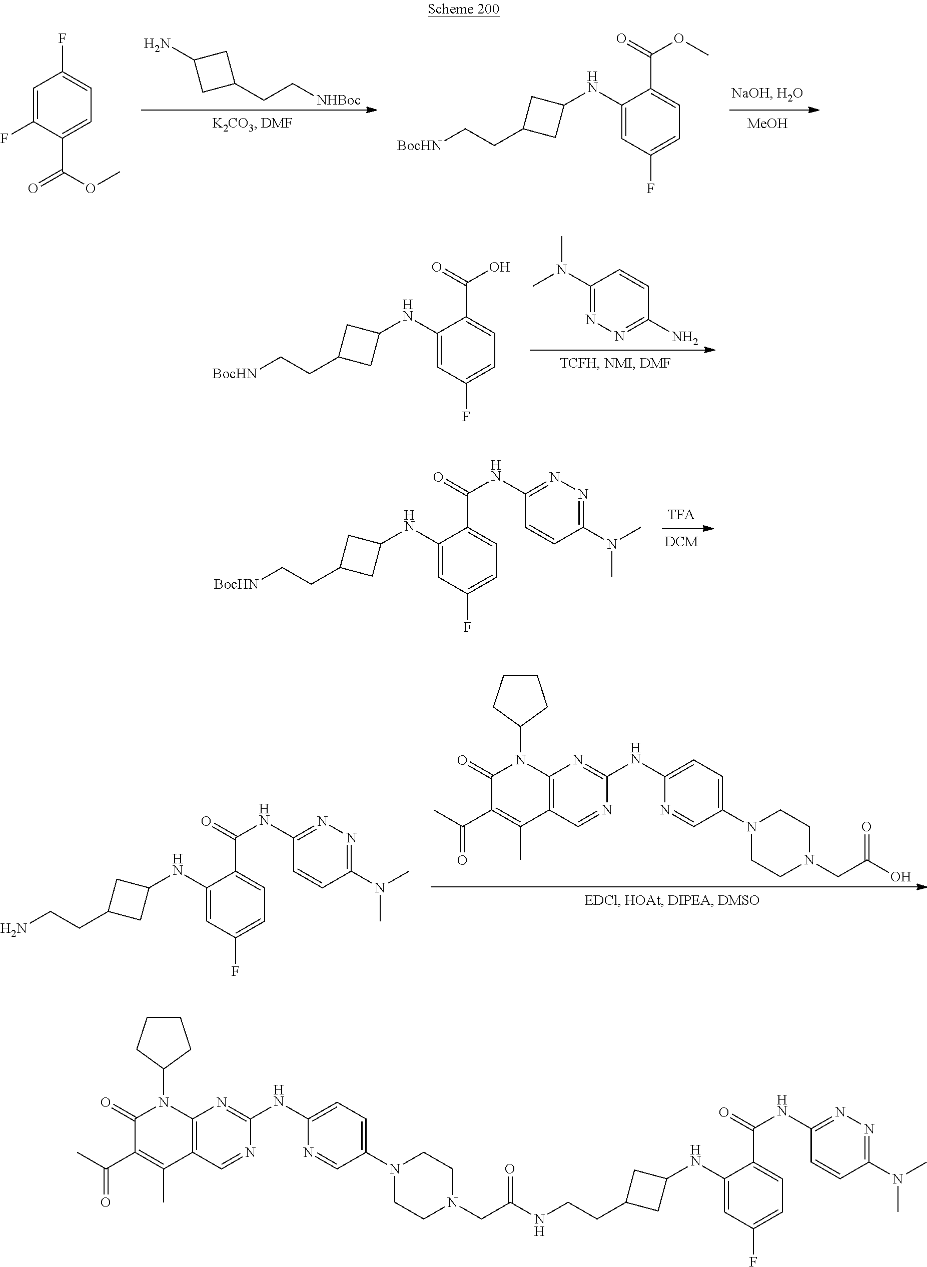

CPD-258 was synthesized following the procedures for preparing CPD-255 (12.9 mg, 3% yield over 5 steps) as a light-yellow solid. MS (ESI) m/z=860.8 [M+H]⁺.

### Example 201. 2-((7-(2-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)acetamido)heptyl)amino)-N-(6-(dimethylamino)pyridazin-3-yl)-6-(methylamino)nicotinamide (CPD-143)

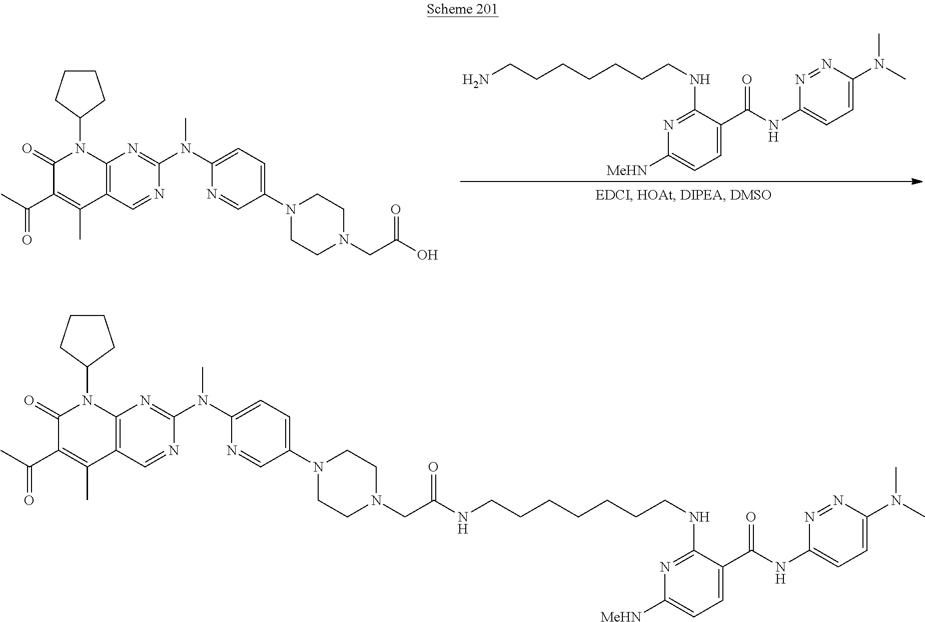 [SMILES: CC(=O)c1c(C)c2cnc(N(C)c3ccc(N4CCN(CC(=O)O)CC4)cn3)nc2n(C2CCCC2)c1=O ; CNc1ccc(C(=O)Nc2ccc(N(C)C)nn2)c(NCCCCCCCN)n1 ; CNc1ccc(C(=O)Nc2ccc(N(C)C)nn2)c(NCCCCCCCNC(=O)CN2CCN(c3ccc(N(C)c4ncc5c(C)c(C(C)=O)c(=O)n(C6CCCC6)c5n4)nc3)CC2)n1]

A mixture of 2-(7-aminoheptylamino)-N-[6-(dimethylamino)pyridazin-3-yl]-6-(methylamino)pyridine-3-carboxamide (5 mg, 9.72 μmol), 2-[4-[6-[(6-acetyl-8-cyclopentyl-5-methyl-7-oxo-pyrido[2,3-d]pyrimidin-2-yl)-methyl-amino]-3-pyridyl]piperazin-1-yl]acetic acid (5.05 mg, 9.72 μmol), EDCI (2.79 mg, 14.58 μmol), HOAt (3.31 mg, 14.58 Mol) and DIPEA (6.28 mg, 48.59 μmol) in DMSO (2 mL) was stirred at rt for 16 h. The reaction mixture was poured into water (20 mL) and extracted with ethyl acetate (3×20 mL). The combined organic layers were washed with saturated brine (20 mL), dried over anhydrous sodium sulfate, filtered, and evaporated under reduced pressure. The resulting residue was purified by reverse-phase chromatography and pre-HPLC to give the desired product (2.2 mg, 22% yield) as a yellow solid. MS (ESI) m/z=902.8 [M+H]⁺.

### Example 202. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-y)propanoyl)-2-azaspiro[3.3]heptan-6-yl)amino)-N-(1-methyl-1H-pyrazol-3-yl)-6-(methylamino)nicotinamide (CPD-144)

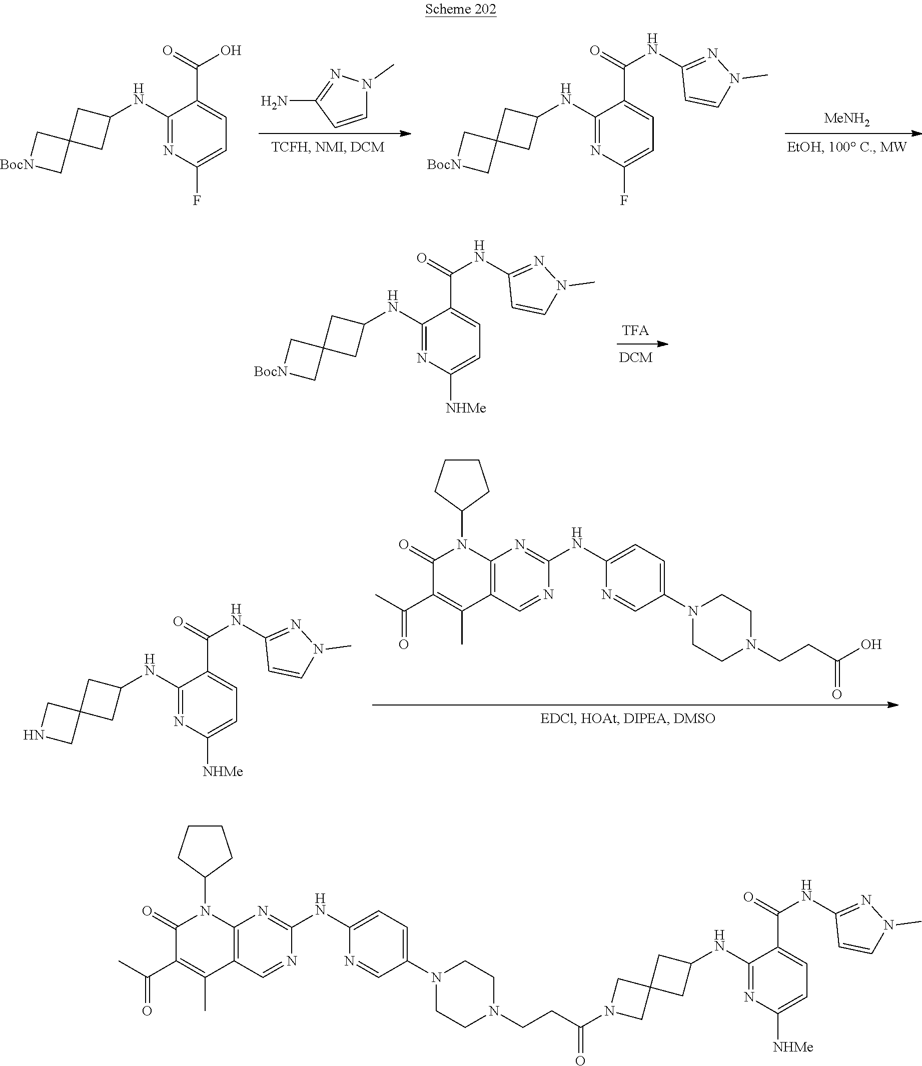

### Step 1. Synthesis of tert-butyl 6-((6-fluoro-3-((1-methyl-1H-pyrazol-3-yl)carbamoyl)pyridin-2-yl)amino)-2-azaspiro[3.3]heptane-2-carboxylate

To a mixture of 2-((2-(tert-butoxycarbonyl)-2-azaspiro[3.3]heptan-6-yl)amino)-6-fluoronicotinic acid (30 mg, 85.4 μmol) and 1-methyl-1H-pyrazol-3-amine (8.3 mg, 85.4 μmol) in dichloromethane (5 mL) were added NMI (21.0 mg, 0.256 mmol) and TCFH (47.8 mg, 0.171 mmol). The mixture was stirred at rt for 2 h, before it was concentrated and purified by flash chromatography on silica gel (petroleum ether/ethyl acetate=5:1) to give the desired product (30 mg, 82% yield) as a white solid. MS (ESI) m/z=431.2 [M+H]⁺.

### Step 2. Synthesis of tert-butyl 6-((3-((1-methyl-1H-pyrazol-3-yl)carbamoyl)-6-(methylamino)pyridin-2-yl)amino)-2-azaspiro[3.3]heptane-2-carboxylate

A mixture of tert-butyl 6-((6-fluoro-3-((1-methyl-1H-pyrazol-3-yl)carbamoyl)pyridin-2-yl)amino)-2-azaspiro[3.3]heptane-2-carboxylate (30.0 mg, 69.7 μmol) in methanamine solution (1 mL, 30% in EtOH) was heated to 100° C. under microwave irradiation for 1 h. After cooling down to rt, the mixture was concentrated and purified by flash chromatography on silica gel (petroleum ether/ethyl acetate=3:1) to give the desired product (24 mg, 78% yield) as a yellow solid. MS (ESI) m/z=442.2 [M+H]⁺.

### Step 3. Synthesis of 2-((2-azaspiro[3.3]heptan-6-yl)amino)-N-(1-methyl-1H-pyrazol-3-yl)-6-(methylamino)nicotinamide

To a solution of tert-butyl 6-((3-((1-methyl-1H-pyrazol-3-yl)carbamoyl)-6-(methylamino)pyridin-2-yl)amino)-2-azaspiro[3.3]heptane-2-carboxylate (20 mg, 45.3 gmol) in dichloromethane (5 mL) was added TFA (1.5 mL). After the reaction mixture was stirred at rt for 1 h, it was concentrated to give the crude product (14 mg, 91% yield) as a yellow oil. This compound was used directly in the next step without further purification. MS (ESI) m/z=342.2 [M+H]⁺.

### Step 4. Synthesis of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1-methyl-1H-pyrazol-3-yl)-6-(methylamino)nicotinamide

To a mixture of 2-((2-azaspiro[3.3]heptan-6-yl)amino)-N-(1-methyl-1H-pyrazol-3-yl)-6-(methylamino)nicotinamide (7.0 mg, 20.5 μmol), DIPEA (26.5 mg, 205.0 μmol) and HOAt (5.6 mg, 41.0 μmol) in DMSO (3 mL) were added EDCI (7.9 mg, 41.0 μmol) and 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoic acid (10.7 mg, 20.5 μmol). After the reaction mixture was stirred at rt for 3 h, it was purified by prep-HPLC to provide the desired product (5 mg, 29% yield) as a yellow solid. MS (ESI) m/z=843.8 [M+H]⁺.

### Example 203. 2-(((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)methyl)amino)-N-(6-(dimethylamino)pyridazin-3-yl)-6-(methylamino)nicotinamide (CPD-145)

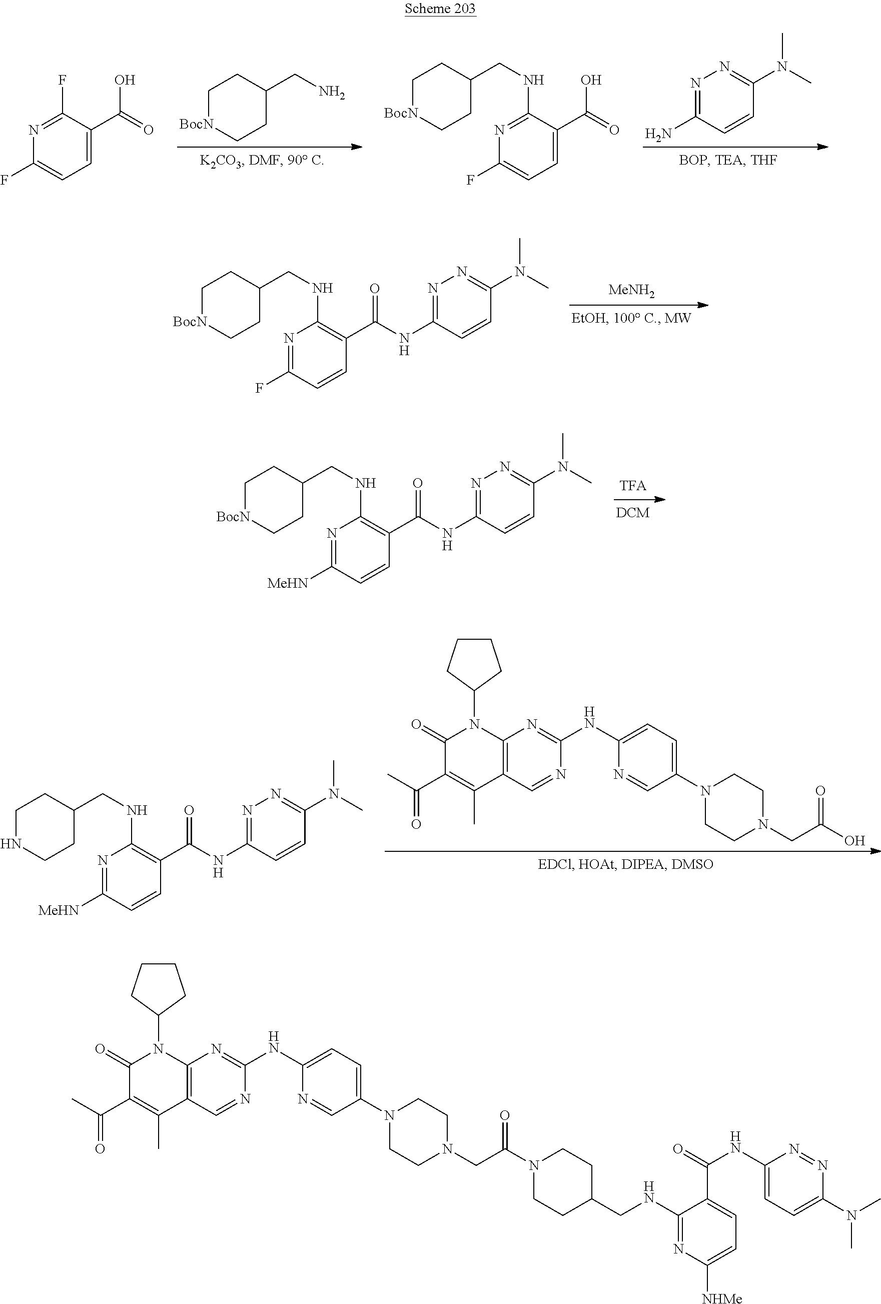

### Step 1. Synthesis of 2-(((1-(tert-butoxycarbonyl)piperidin-4-yl)methyl)amino)-6-fluoronicotinic acid

To a solution of 2,6-difluoronicotinic acid (100 mg, 0.628 mmol) and tert-butyl 4-(aminomethyl)piperidine-1-carboxylate (134.7 mg, 0.628 mmol) in DMF (5 mL) was added K₂CO₃ (260.2 mg, 1.89 mmol) at rt. The reaction mixture was stirred at 90 [symbol] for 10 h. After cooling down to rt, it was filtered and purified by reverse-phase chromatography to give the desired product (90 mg, 41% yield) as a white solid. MS (ESI) m/z=354.2 [M+H]⁺.

### Step 2. Synthesis of tert-butyl 4-(((3-((6-(dimethylamino)pyridazin-3-yl)carbamoyl)-6-fluoropyridin-2-yl)amino)methyl)piperidine-1-carboxylate

To a mixture of 2-(((1-(tert-butoxycarbonyl)piperidin-4-yl)methyl)amino)-6-fluoronicotinic acid (90 mg, 0.255 mmol) and N³,N³-dimethylpyridazine-3,6-diamine (35.2 mg, 0.255 mmol) in THF (4 mL) were added BOP (104.0 mg, 0.509 mmol) and TEA (257.7 mg, 2.55 mmol). The mixture was stirred at 50 [symbol] for 10 h. After cooling down to rt, the mixture was concentrated and purified by flash chromatography on silica gel (petroleum ether/ethyl acetate=3:1) to provide the desired product (50 mg, 41% yield) as a yellow solid. MS (ESI) m/z=474.3 [M+H]⁺.

The remaining steps were performed following the standard procedures for preparing CPD-144 to provide the desired product (3.5 mg, 10% yield over 3 steps) as a light-yellow solid. MS (ESI) m/z=872.7 [M+H]⁺.

### Example 204. 2-(((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)methyl)amino)-N-(6-(dimethylamino)pyridazin-3-yl)-6-(methylamino)nicotinamide (CPD-146)

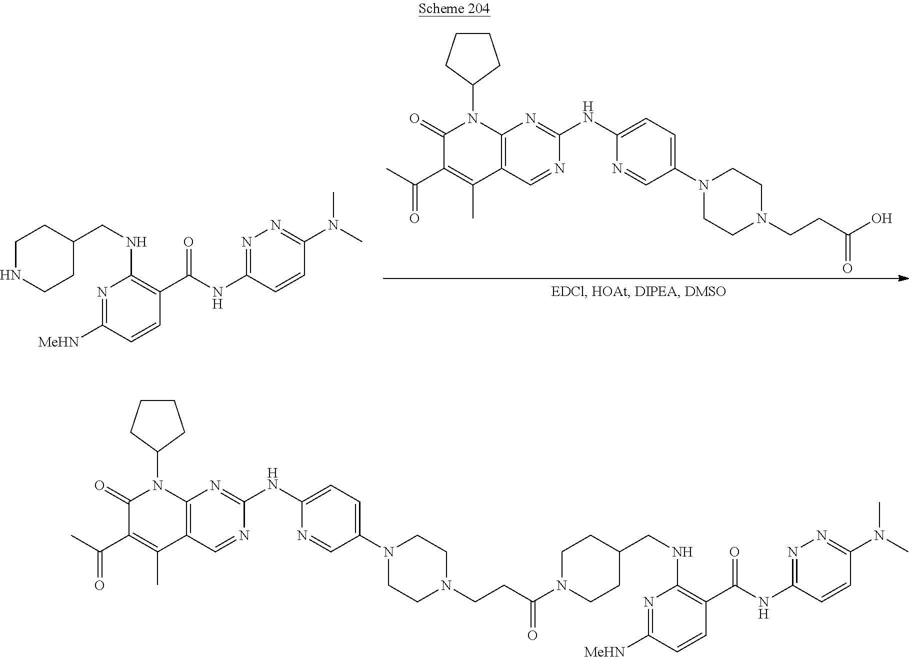 [SMILES: CNc1ccc(C(=O)Nc2ccc(N(C)C)nn2)c(NCC2CCNCC2)n1 ; CC(=O)c1c(C)c2cnc(Nc3ccc(N4CCN(CCC(=O)O)CC4)cn3)nc2n(C2CCCC2)c1=O ; CNc1ccc(C(=O)Nc2ccc(N(C)C)nn2)c(NCC2CCN(C(=O)CCN3CCN(c4ccc(Nc5ncc6c(C)c(C(C)=O)c(=O)n(C7CCCC7)c6n5)nc4)CC3)CC2)n1]

CPD-146 was synthesized following the standard procedure for preparing CPD-143 (1.2 mg, 5% yield) as a light-yellow solid. MS (ESI) m/z=886.8 [M+H]⁺.

### Example 205. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)spiro[3.3]heptan-2-yl)amino)-N-(6-(dimethylamino)pyridazin-3-yl)-6-(methylamino)nicotinamide (CPD-147)

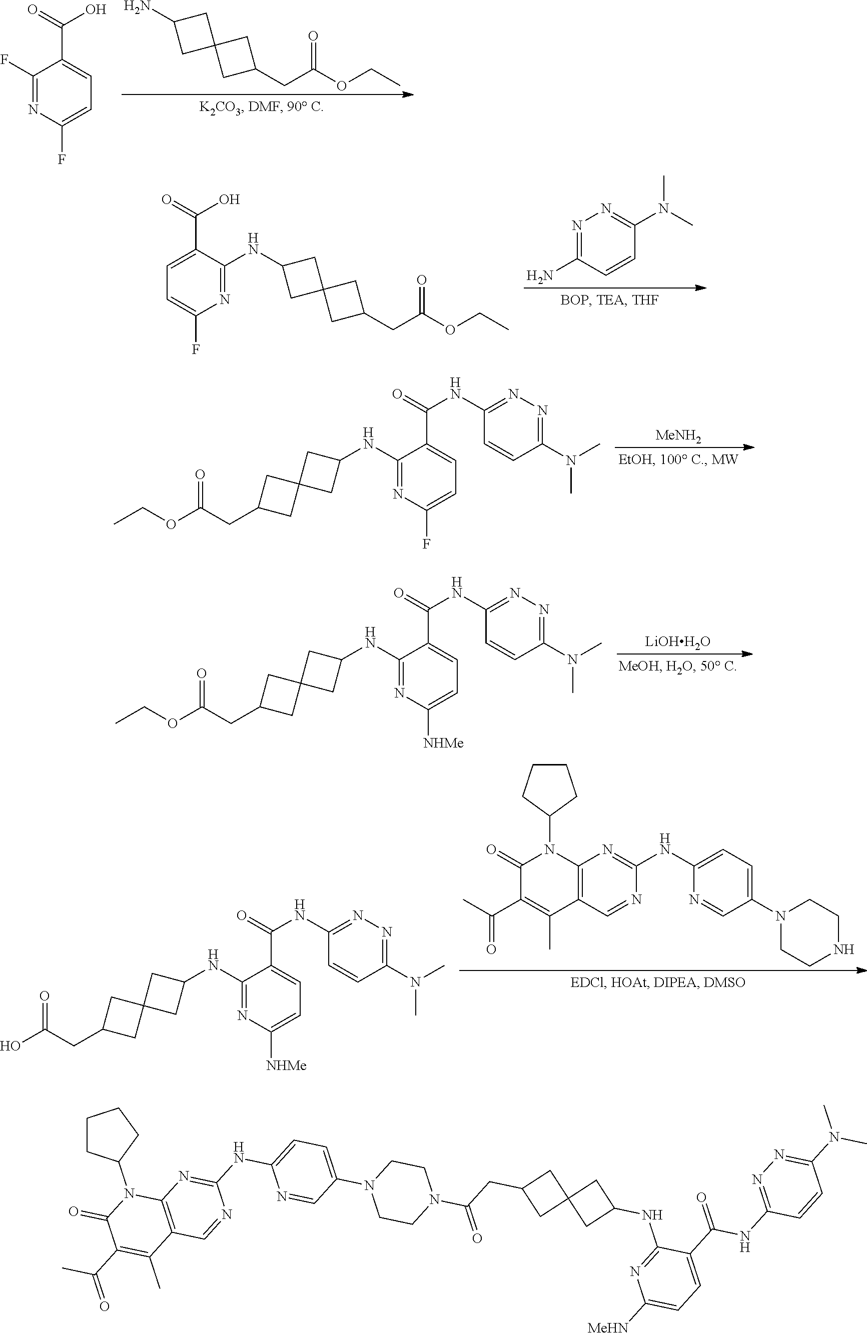

CPD-147 was synthesized following the standard procedures for preparing CPD-82 (11 mg, 7% yield over 5 steps) as a light-yellow solid. MS (ESI) m/z=869.8 [M+H]⁺.

### Example 206. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethyl)cyclobutyl)amino)-N-(6-(dimethylamino)pyridazin-3-yl)-6-(methylamino)nicotinamide (CPD-148)

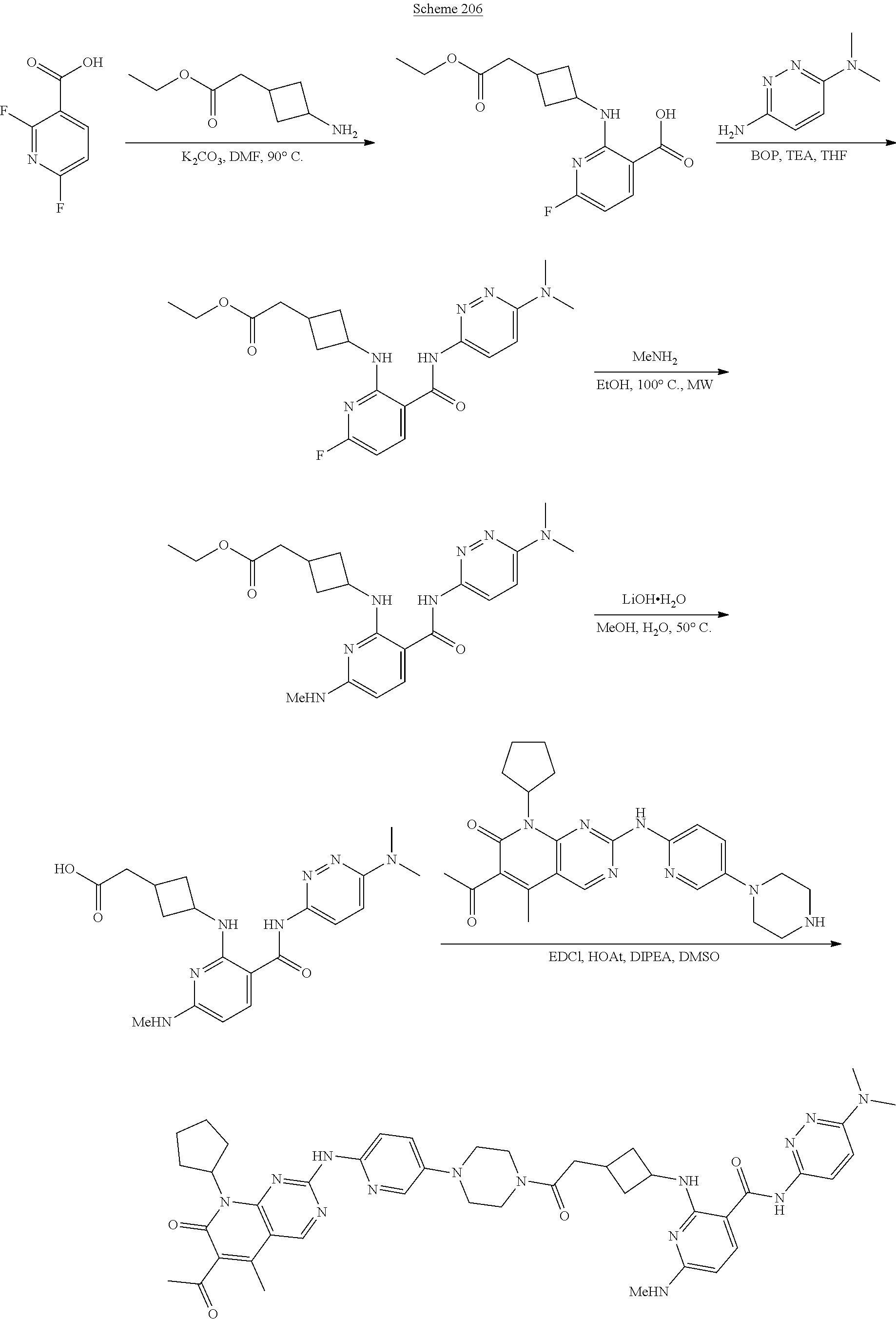

CPD-148 was synthesized following the standard procedures for preparing CPD-82 (11 mg, 5% yield over 5 steps) as a light-yellow solid. MS (ESI) m/z=829.8 [M+H]⁺.

### Example 207. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-(methylamino)nicotinamide (CPD-149)

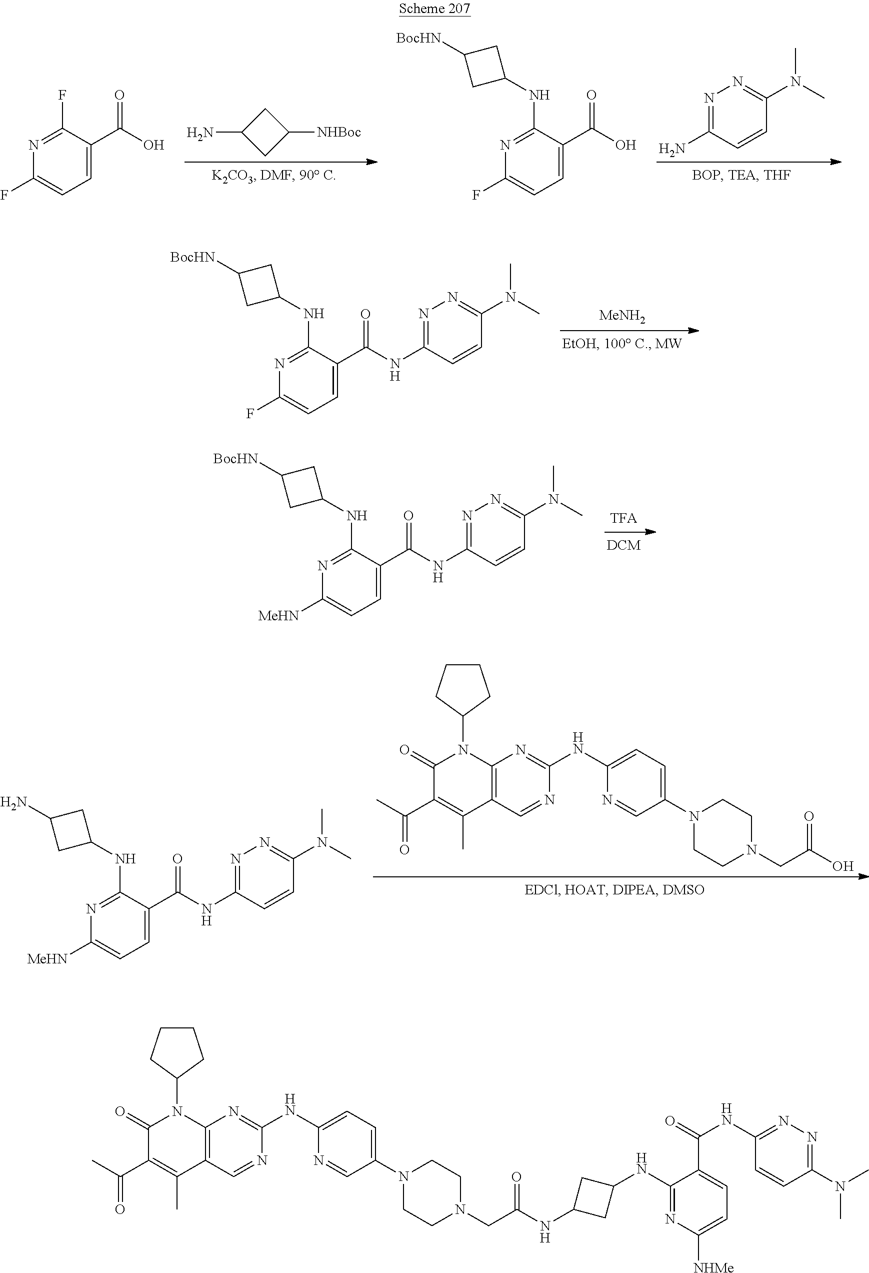

CPD-149 was synthesized following the standard procedures for preparing CPD-145 (12.2 mg, 7% yield over 5 steps) as a light-yellow solid. MS (ESI) m/z=844.7 [M+H]⁺.

### Example 208. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(methylamino)nicotinamide (CPD-150)

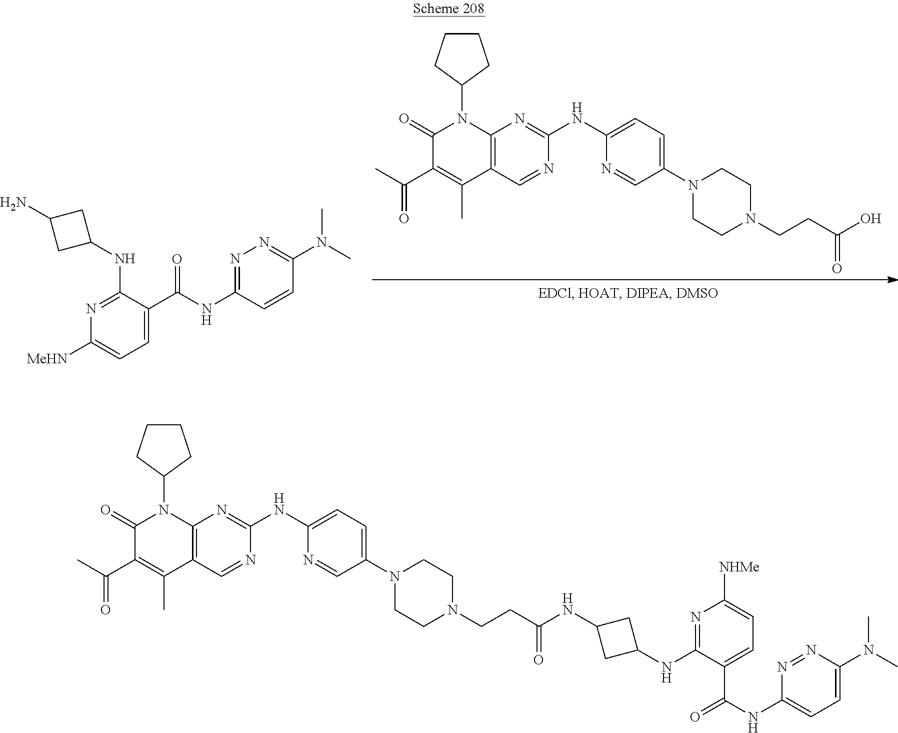 [SMILES: CNc1ccc(C(=O)Nc2ccc(N(C)C)nn2)c(NC2CC(N)C2)n1 ; CC(=O)c1c(C)c2cnc(Nc3ccc(N4CCN(CCC(=O)O)CC4)cn3)nc2n(C2CCCC2)c1=O ; CNc1ccc(C(=O)Nc2ccc(N(C)C)nn2)c(NC2CC(NC(=O)CCN3CCN(c4ccc(Nc5ncc6c(C)c(C(C)=O)c(=O)n(C7CCCC7)c6n5)nc4)CC3)C2)n1]

CPD-150 was synthesized following the standard procedure for preparing CPD-143 (7.8 mg, 28% yield) as a yellow solid. MS (ESI) m/z=858.6 [M+H]⁺.

### Example 209. 4-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(methylamino)pyridin-2-yl)amino)cyclobutyl)piperazine-1-carboxamide (CPD-151)

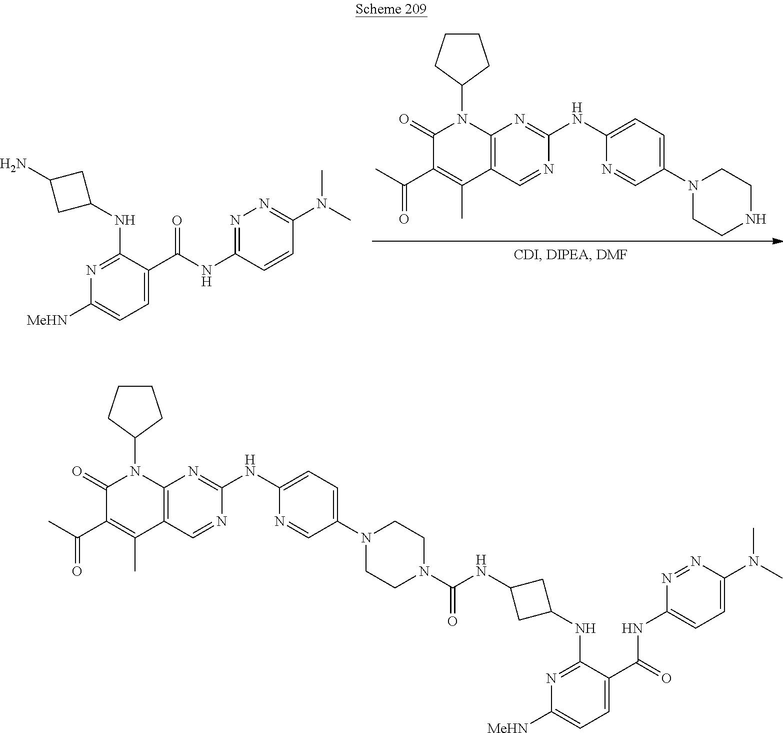 [SMILES: CNc1ccc(C(=O)Nc2ccc(N(C)C)nn2)c(NC2CC(N)C2)n1 ; CC(=O)c1c(C)c2cnc(Nc3ccc(N4CCNCC4)cn3)nc2n(C2CCCC2)c1=O ; CNc1ccc(C(=O)Nc2ccc(N(C)C)nn2)c(NC2CC(NC(=O)N3CCN(c4ccc(Nc5ncc6c(C)c(C(C)=O)c(=O)n(C7CCCC7)c6n5)nc4)CC3)C2)n1]

A mixture of 6-acetyl-8-cyclopentyl-5-methyl-2-((5-(piperazin-1-yl)pyridin-2-yl)amino)pyrido[2,3-d]pyrimidin-7(8H)-one (25 mg, 0.04 mmol), 2-((3-aminocyclobutyl)amino)-N-(6-(dimethylamino)pyridazin-3-yl)-6-(methylamino)nicotinamide (10 mg, 0.02 mmol), CDI (4 mg, 0.02 mmol) and DIPEA (4 mg, 0.03 mmol) in DMF (2 mL) was stirred at rt for 16 h. The reaction mixture was poured into water (10 mL) and extracted with ethyl acetate (3×10 mL). The combined organic layers were washed with saturated brine (15 mL), dried over anhydrous sodium sulfate, filtered, and evaporated under reduced pressure. The resulting residue was purified by reverse-phase chromatography to give the desired product (13.6 mg, 510% yield) as a yellow solid. MS (ESI) m/z=830.8 [M+H]⁺.

### Example 210. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-imidazol-4-yl)-6-(methylamino)nicotinamide (CPD-152)

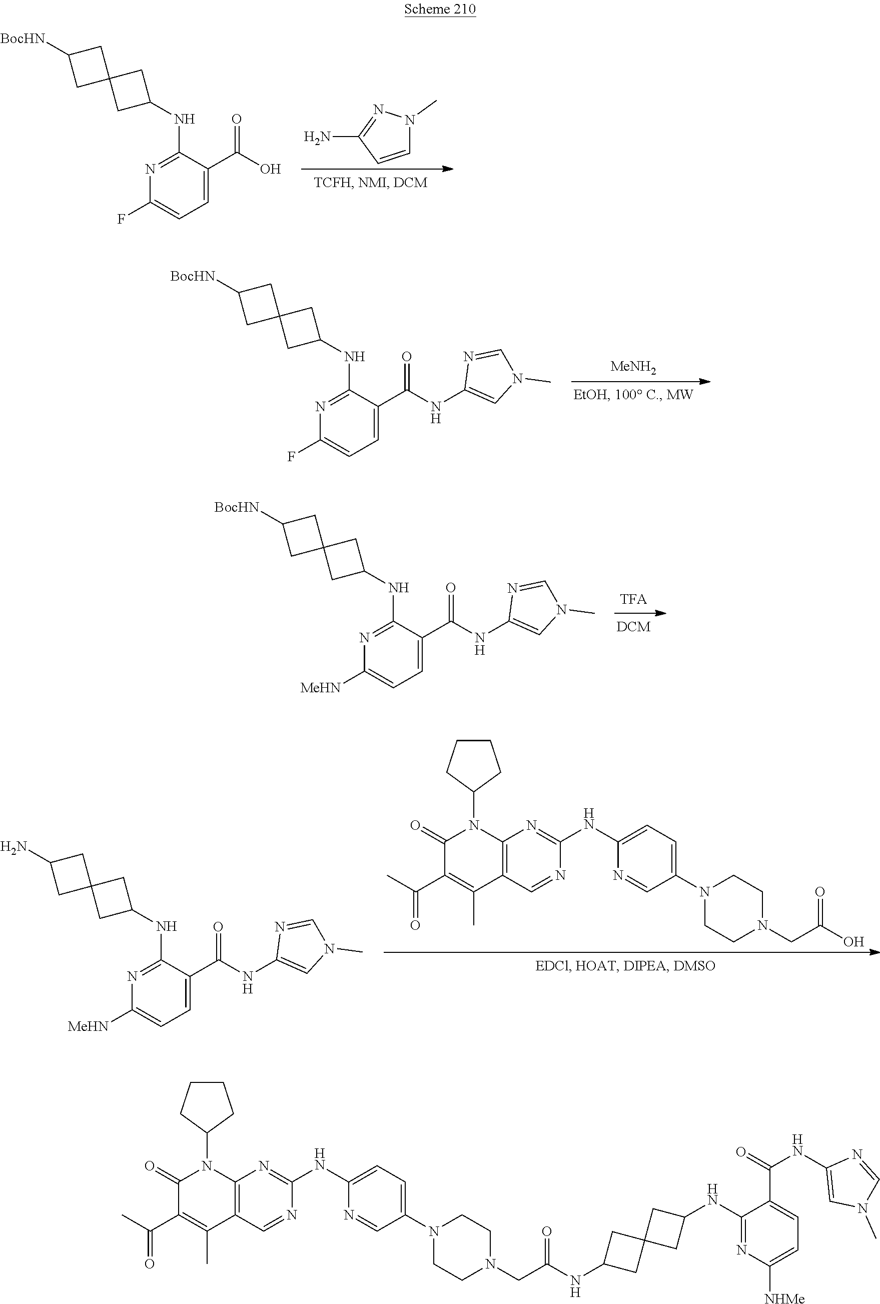

CPD-152 was synthesized following the standard procedures for preparing CPD-144 (21.2 mg, 46% yield over 4 steps) as a yellow solid. MS (ESI) m/z=843.8 [M+H]⁺.

### Example 211. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrazol-5-yl)-6-(methylamino)nicotinamide (CPD-153)

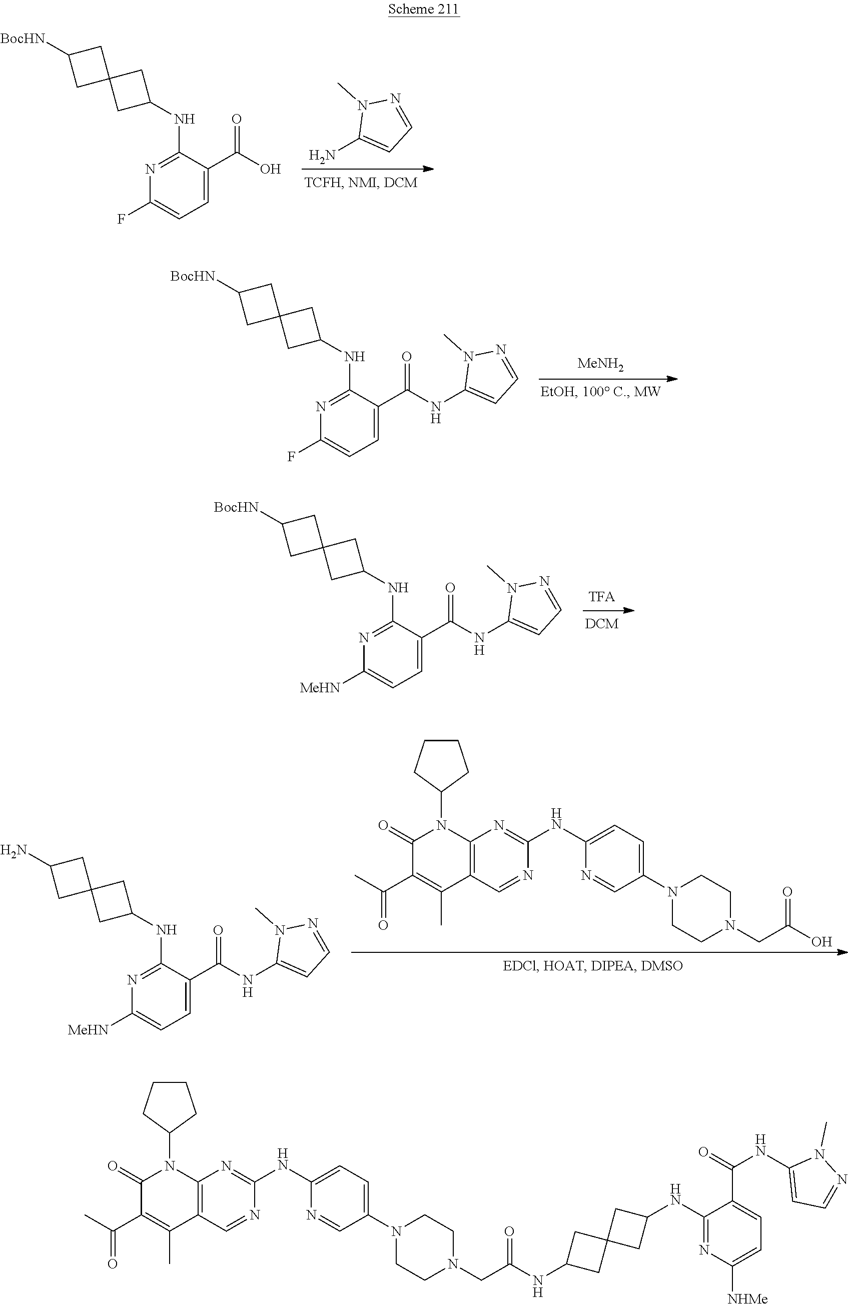

CPD-153 was synthesized following the standard procedures for preparing CPD-144 (13.7 mg, 29% yield over 4 steps) as a yellow solid. MS (ESI) m/z=843.9 [M+H]⁺.

### Example 212. 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-cyclopropylpyridin-2-yl)-6-(methylamino)nicotinamide (CPD-154)

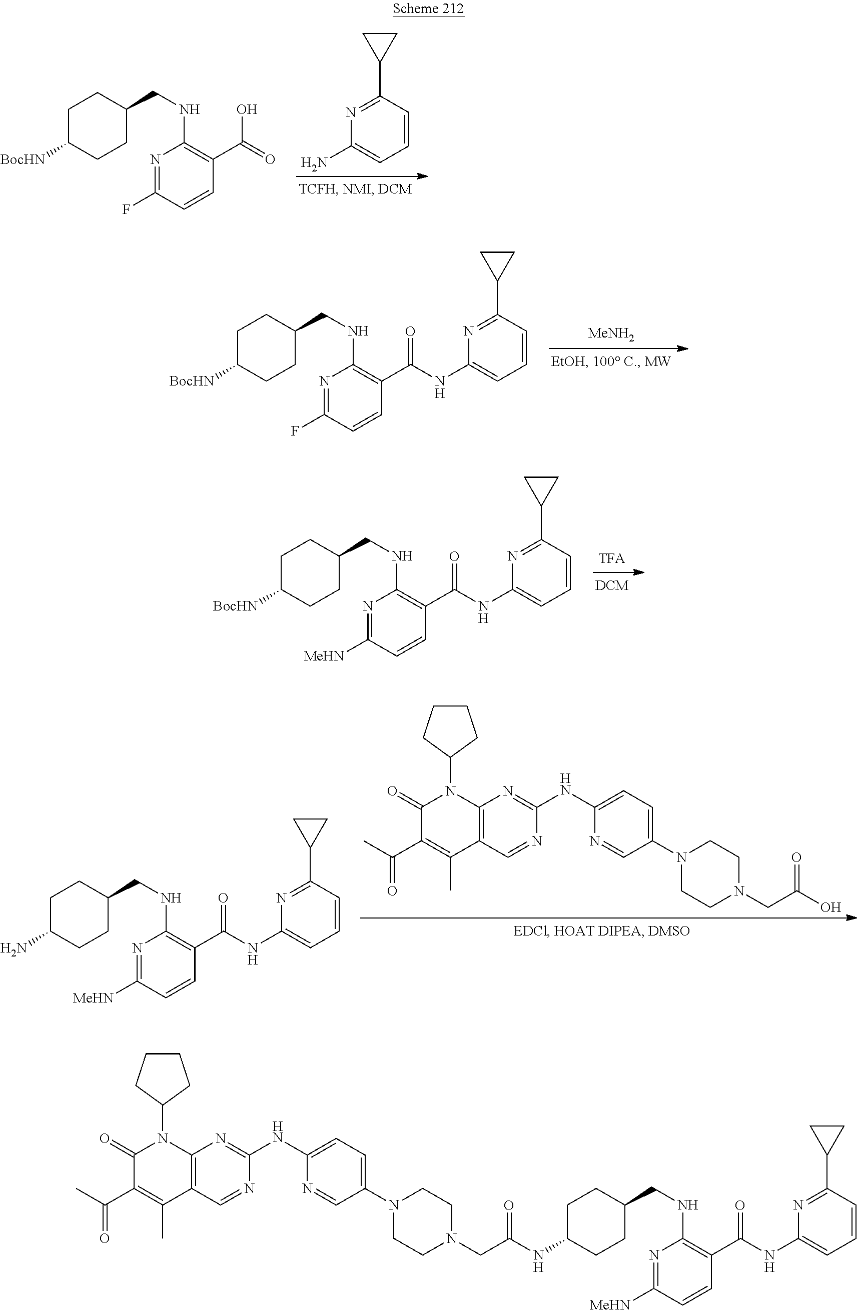

CPD-154 was synthesized following the standard procedures for preparing CPD-144 (1.6 mg, 7% yield over 4 steps) as a yellow solid. MS (ESI) m/z=882.7 [M+H]⁺.

### Example 213. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-6-(methylamino)-N-(6-methylpyridin-2-yl)nicotinamide (CPD-155)

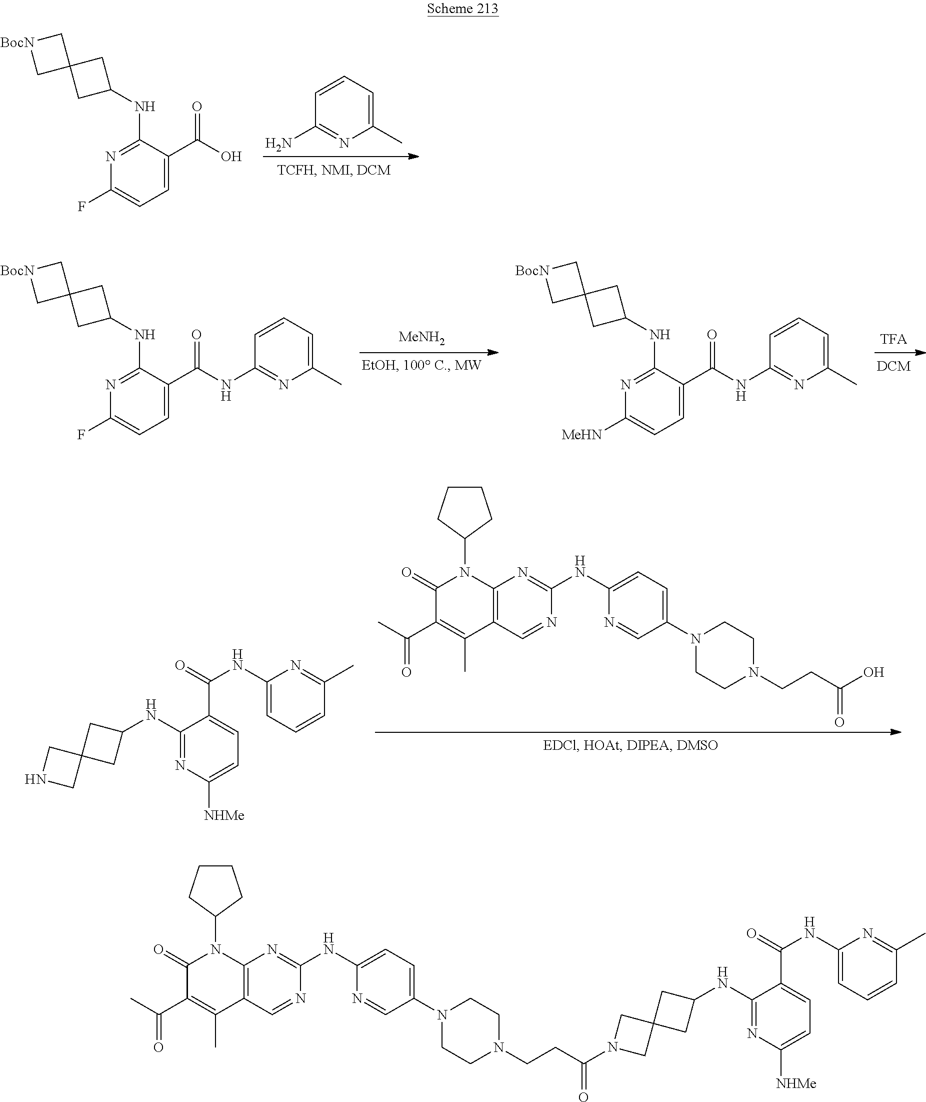

CPD-155 was synthesized following the standard procedures for preparing CPD-144 (5.3 mg, 4% yield over 4 steps) as a yellow solid. MS (ESI) m/z=854.7 [M+H]⁺.

### Example 214. 2-((3-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4-oxobutyl)cyclobutyl)amino)-N-(6-(dimethylamino)pyridazin-3-yl)-6-(methylamino)nicotinamide (CPD-156)

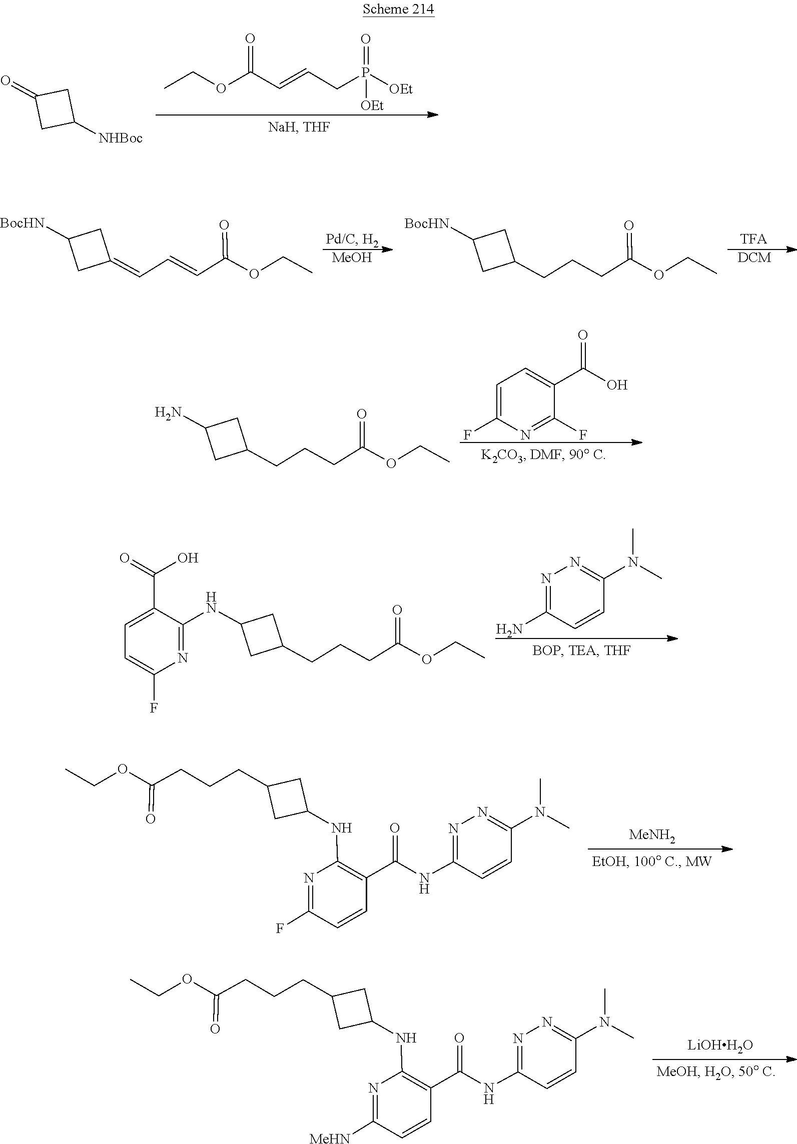

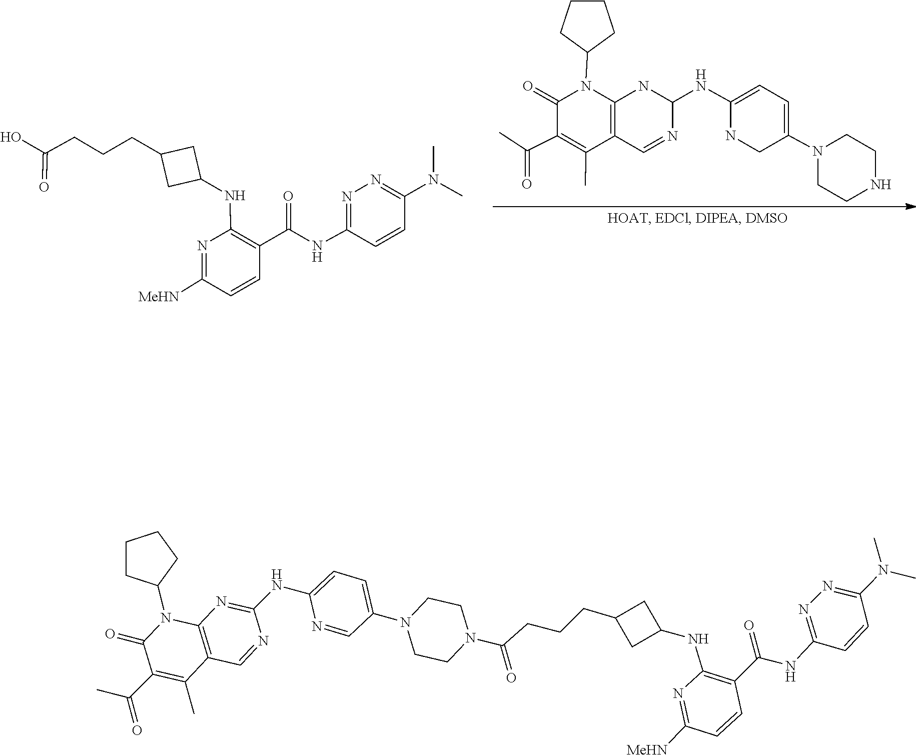

CPD-156 was synthesized following the standard procedures for preparing CPD-82 (16 mg, 2% yield over 8 steps) as a yellow solid. MS (ESI) m/z=857.7 [M+H]⁺.

### Example 215. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamoyl)cyclobutyl)amino)-N-(6-(dimethylamino)pyridazin-3-yl)-6-(methylamino)nicotinamide (CPD-157)

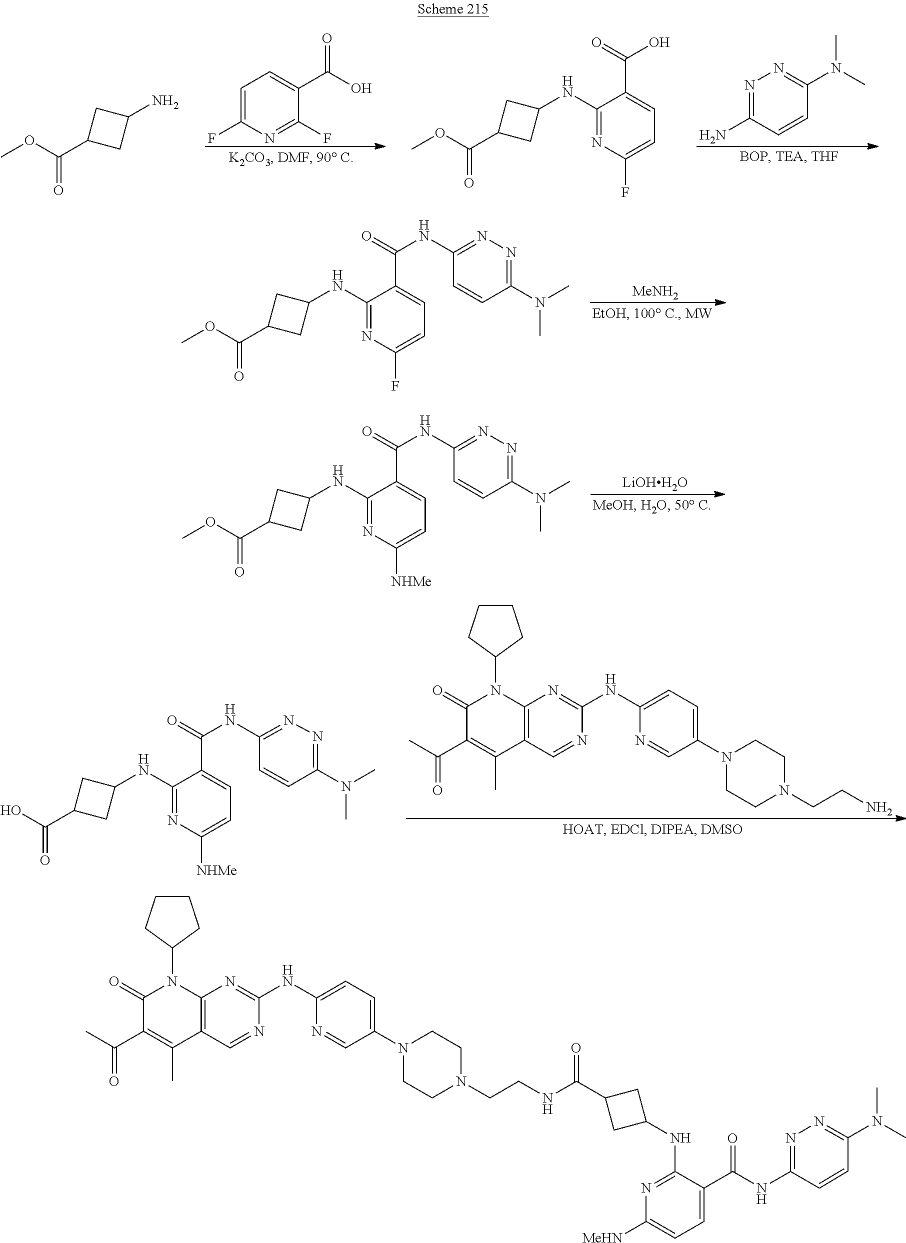

CPD-157 was synthesized following the standard procedures for preparing CPD-82 (16 mg, 2% yield over 5 steps) as a yellow solid. MS (ESI) m/z=858.8 [M+H]⁺.

### Example 216. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.4]octan-6-yl)amino)-N-(6-(dimethylamino)pyridazin-3-yl)-6-(methylamino)nicotinamide (CPD-158)

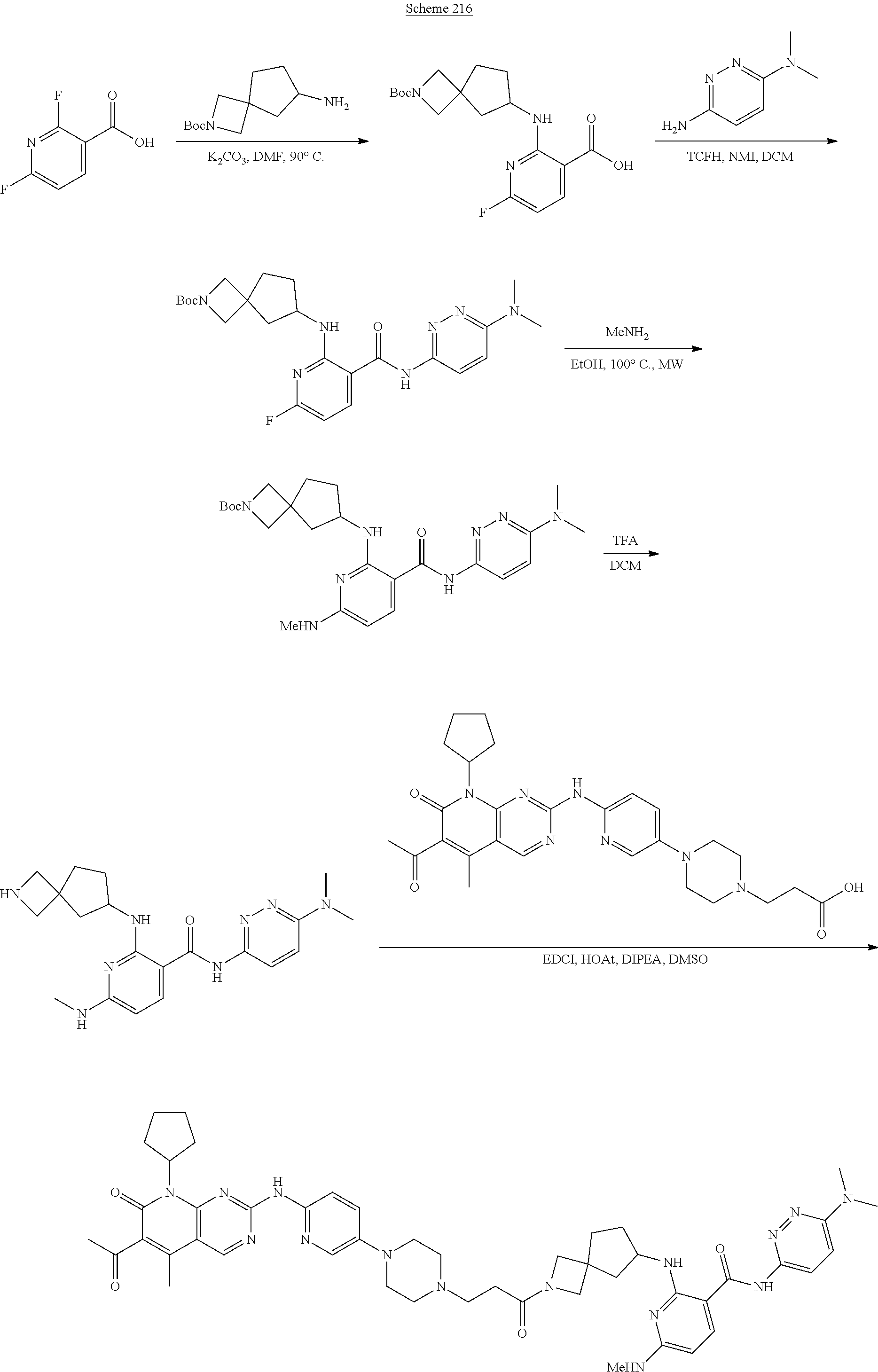

CPD-158 was synthesized following the standard procedures for preparing CPD-144 (8.8 mg, 8% yield over 5 steps) as a yellow solid. MS (ESI) m/z=898.9 [M+H]⁺.

### Example 217. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(isoxazol-3-yl)-6-(methylamino)nicotinamide (CPD-159)

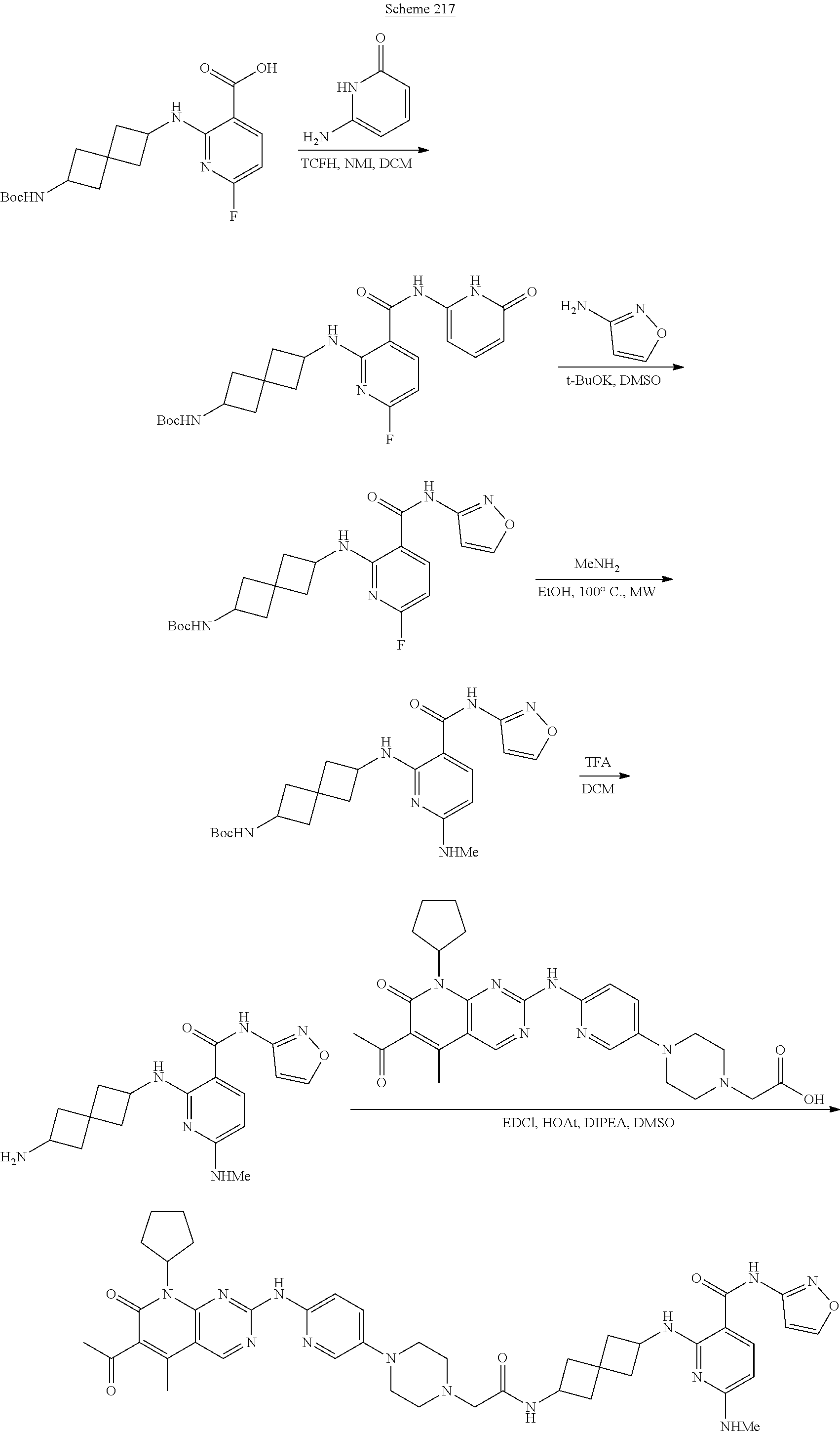

### Step 1. Synthesis of tert-butyl (6-((6-fluoro-3-((6-oxo-1,6-dihydropyridin-2-yl)carbamoyl)pyridin-2-yl)amino)spiro[3.3]heptan-2-yl)carbamate

To a solution of 2-((6-((tert-butoxycarbonyl)amino)spiro[3.3]heptan-2-yl)amino)-6-fluoronicotinic acid (149.85 mg, 410.10 μmol) in dichloromethane (4 mL) were added 6-amino-1H-pyridin-2-one (45.16 mg, 410.10 mol), TCFH (172.24 mg, 615.15 μmol) and NMI (117.84 mg, 1.44 mmol). After the reaction mixture was stirred at rt for 1 h, it was poured into water (30 mL) and extracted with ethyl acetate (3×20 mL). The combined organic layers were washed with saturated brine (15 mL), dried over anhydrous sodium sulfate, filtered, and evaporated under reduced pressure. The resulting residue was purified by reverse-phase chromatography to give the desired product (130 mg, 69% yield) as a pale-white solid. MS (ESI) m/z=458.7 [M+H]⁺.

### Step 2. Synthesis of tert-butyl (6-((6-fluoro-3-(isoxazol-3-ylcarbamoyl)pyridin-2-yl)amino)spiro[3.3]heptan-2-yl)carbamate

To a solution of tert-butyl (6-((6-fluoro-3-((6-oxo-1,6-dihydropyridin-2-yl)carbamoyl)pyridin-2-yl)amino)spiro[3.3]heptan-2-yl)carbamate (60 mg, 131.2 μmol) in DMSO (2 mL) was added isoxazol-3-amine (16.54 mg, 196.7 μmol). The reaction mixture was stirred at rt for 5 min. After this time, the reaction mixture was added t-BuOK solution (1 M in THF, 3.93 mmol, 0.4 mL) and stirred at rt for additional 0.5 h. The reaction mixture was poured into water (20 mL) and extracted with ethyl acetate (3×20 mL). The combined organic layers were washed with saturated brine (15 mL), dried over anhydrous sodium sulfate, filtered, and evaporated under reduced pressure. The resulting residue was purified by reverse-phase chromatography to give the desired product (25 mg, 44% yield) as a pale-white solid. MS (ESI) m/z=432.5 [M+H]⁺.

### Step 3. Synthesis of tert-butyl (6-((3-(isoxazol-3-ylcarbamoyl)-6-(methylamino)pyridin-2-yl)amino)spiro[3.3]heptan-2-yl)carbamate

A solution of tert-butyl (6-((6-fluoro-3-(isoxazol-3-ylcarbamoyl)pyridin-2-yl)amino)spiro[3.3]heptan-2-yl)carbamate (25 mg, 57.94 mol) in MeNH₂ solution (30% in EtOH, 1 mL) was stirred at 100° C. under microwave irradiation for 10 min. After cooling down to rt, the mixture was concentrated and purified by silica gel chromatography to give the desired product (6 mg, 23% yield) as a pale-white solid. MS (ESI) m/z=443.6 [M+H]⁺.

### Step 4. Synthesis of 2-((6-aminospiro[3.3]heptan-2-yl)amino)-N-(isoxazol-3-yl)-6-(methylamino)nicotinamide

A solution of tert-butyl (6-((3-(isoxazol-3-ylcarbamoyl)-6-(methylamino)pyridin-2-yl)amino)spiro[3.3]heptan-2-yl)carbamate (6 mg, 13.6 mol) in dichloromethane (2 mL) and TFA (1 mL) was stirred at rt for 1 h. The reaction mixture was concentrated and purified by reverse-phase chromatography to give the desired product (4.5 mg, 97% yield) as a yellow oil. MS (ESI) m/z=343.5 [M+H]⁺.

### Step 5. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(isoxazol-3-yl)-6-(methylamino)nicotinamide

A mixture of 2-((6-aminospiro[3.3]heptan-2-yl)amino)-N-(isoxazol-3-yl)-6-(methylamino)nicotinamide (4.5 mg, 13.1 μmol), 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (7.97 mg, 15.8 μmol), EDCI (3.78 mg, 19.71 μmol), HOAt (2.68 mg, 19.7 μmol) and DIPEA (8.49 mg, 65.7 μmol) in DMSO (1 mL) was stirred at rt for 16 h. The reaction mixture was purified by prep-HPLC to give the desired product (1.2 mg, 110% yield) as a yellow solid. MS (ESI) m/z=830.8 [M+H]⁺.

### Example 218. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(5-methylisoxazol-3-yl)nicotinamide (CPD-160)

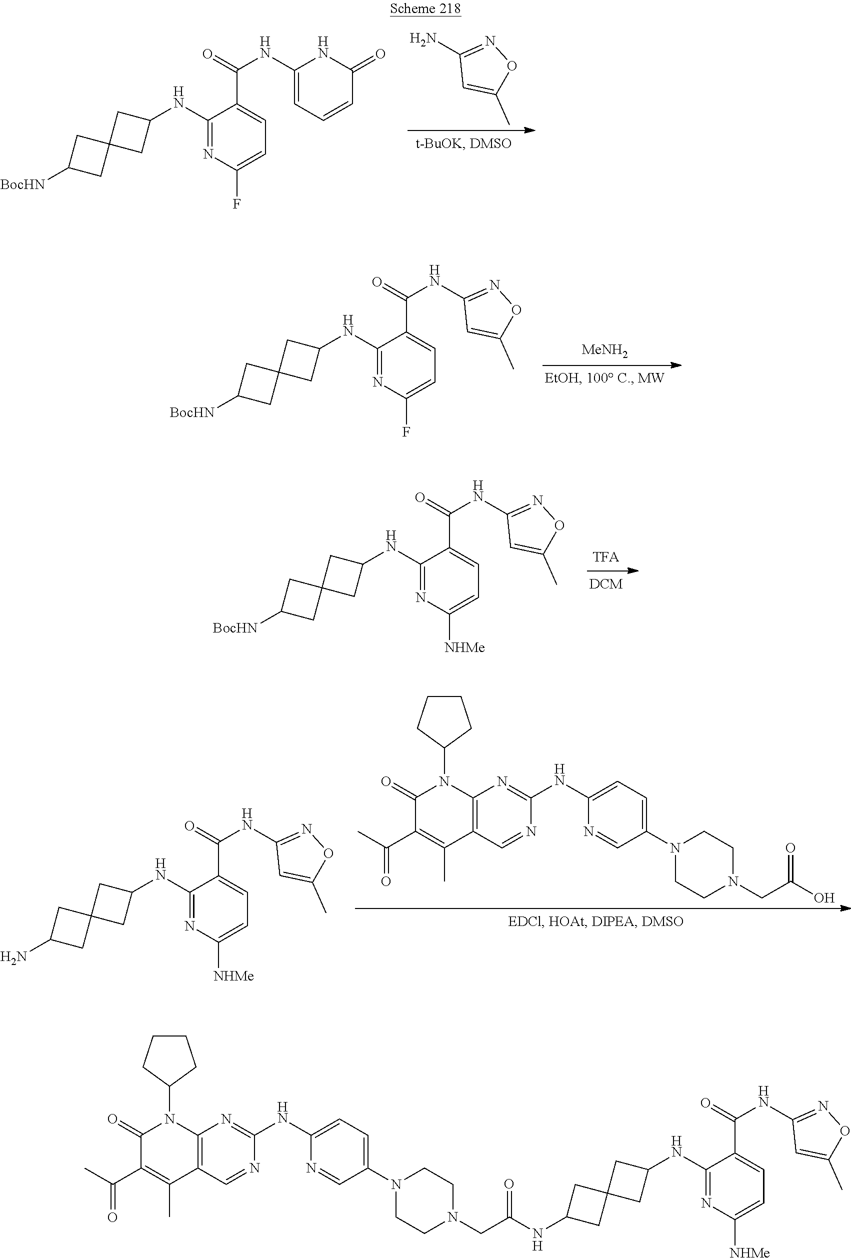

CPD-160 was synthesized following the standard procedures for preparing CPD-159 (7.9 mg, 19% yield over 4 steps) as a yellow solid. MS (ESI) m/z=844.7 [M+H]⁺.

### Example 219. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-methoxypyridin-2-yl)-6-(methylamino)nicotinamide (CPD-161)

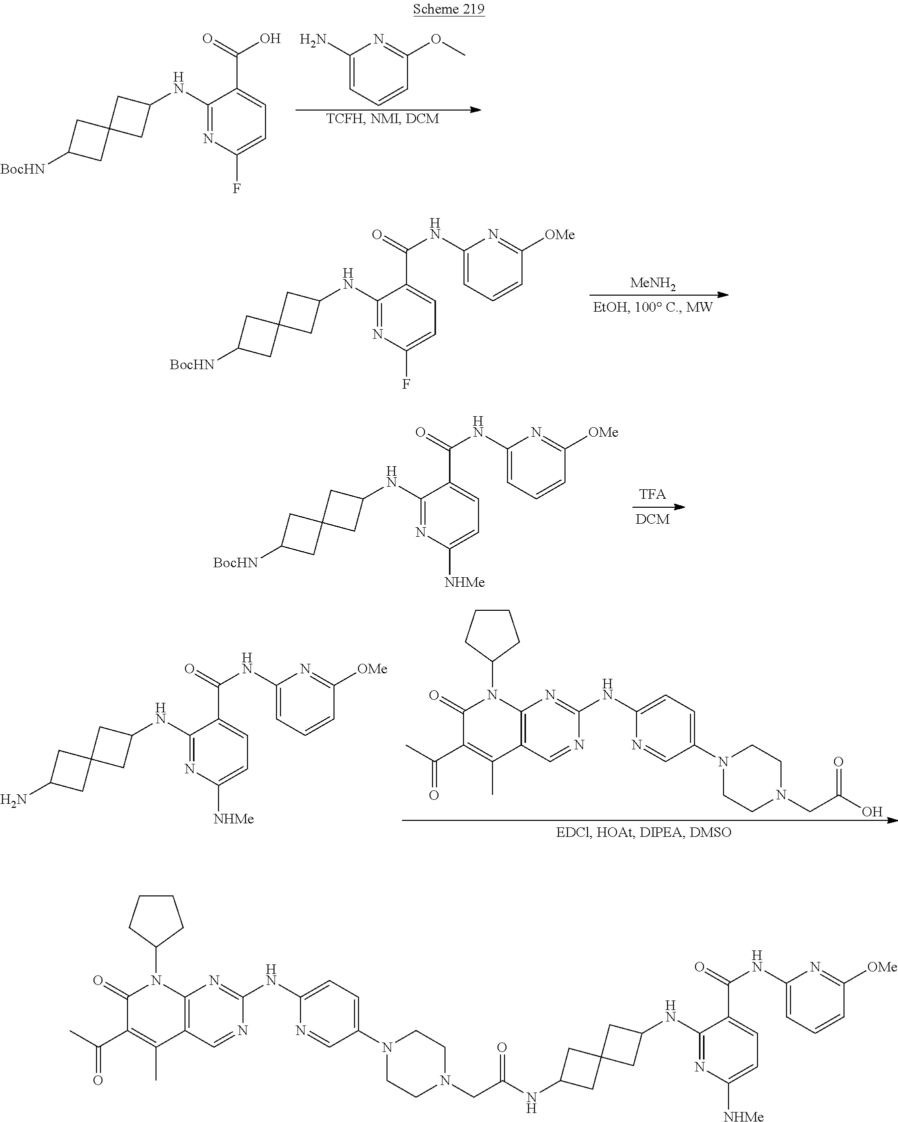

CPD-161 was synthesized following the standard procedures for preparing CPD-144 (1.0 mg, 2% yield over 4 steps) as a yellow solid. MS (ESI) m/z=870.8 [M+H]⁺.

### Example 220. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-cyclopropylpyridin-2-yl)-6-(methylamino)nicotinamide (CPD-162)

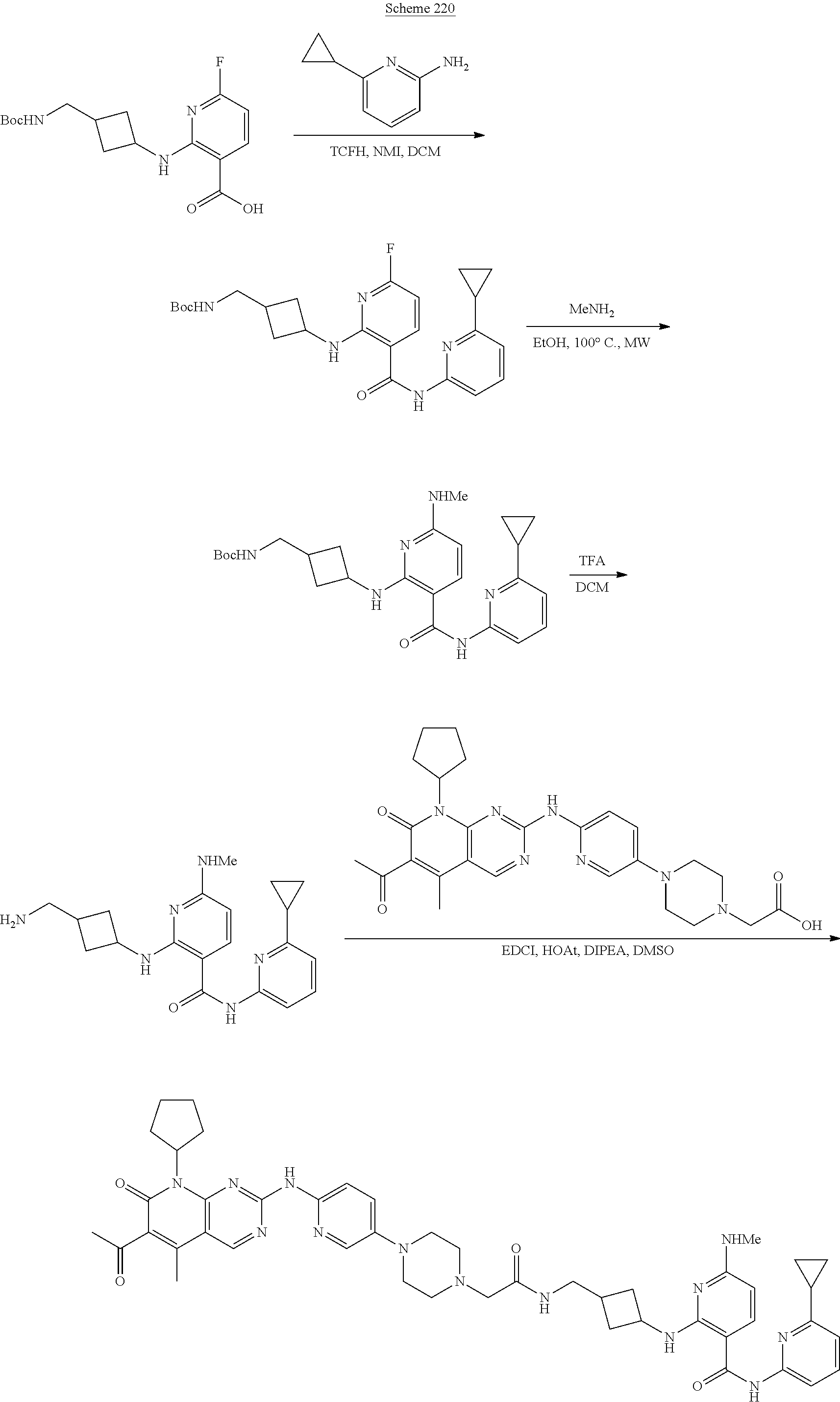

CPD-162 was synthesized following the standard procedures for preparing CPD-144 (3.3 mg, 5% yield over 4 steps) as a yellow solid. MS (ESI) m/z=854.8 [M+H]⁺.

### Example 221. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropylpyridin-2-yl)-6-((methyl-d3)amino)nicotinamide (CPD-163)

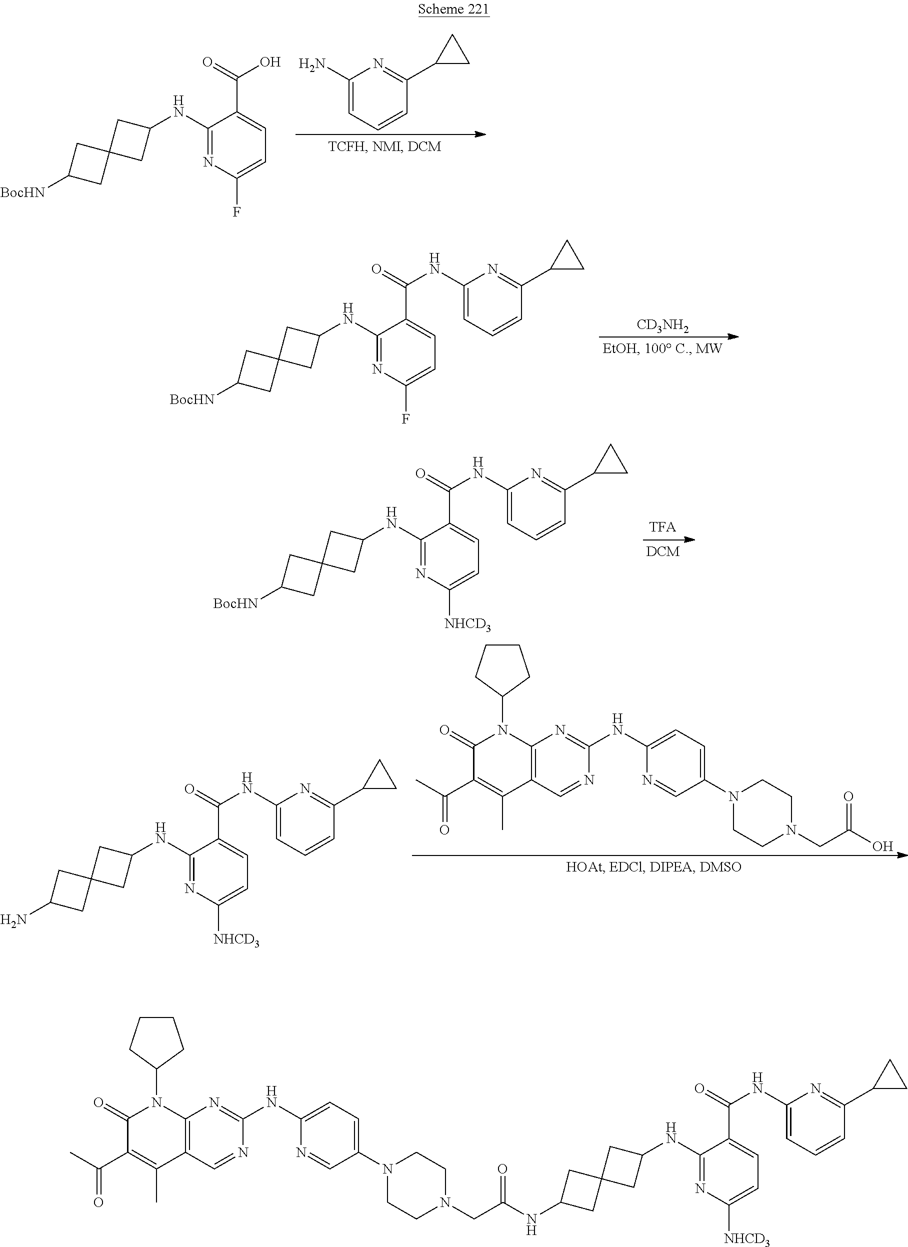

CPD-163 was synthesized following the standard procedures for preparing CPD-144 (1.4 mg, 3% yield over 4 steps) as a yellow solid. MS (ESI) m/z=883.9 [M+H]⁺.

### Example 222. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)methyl)cyclobutyl)amino)-N-(6-(dimethylamino)pyridazin-3-yl)-6-(methylamino)nicotinamide (CPD-164)

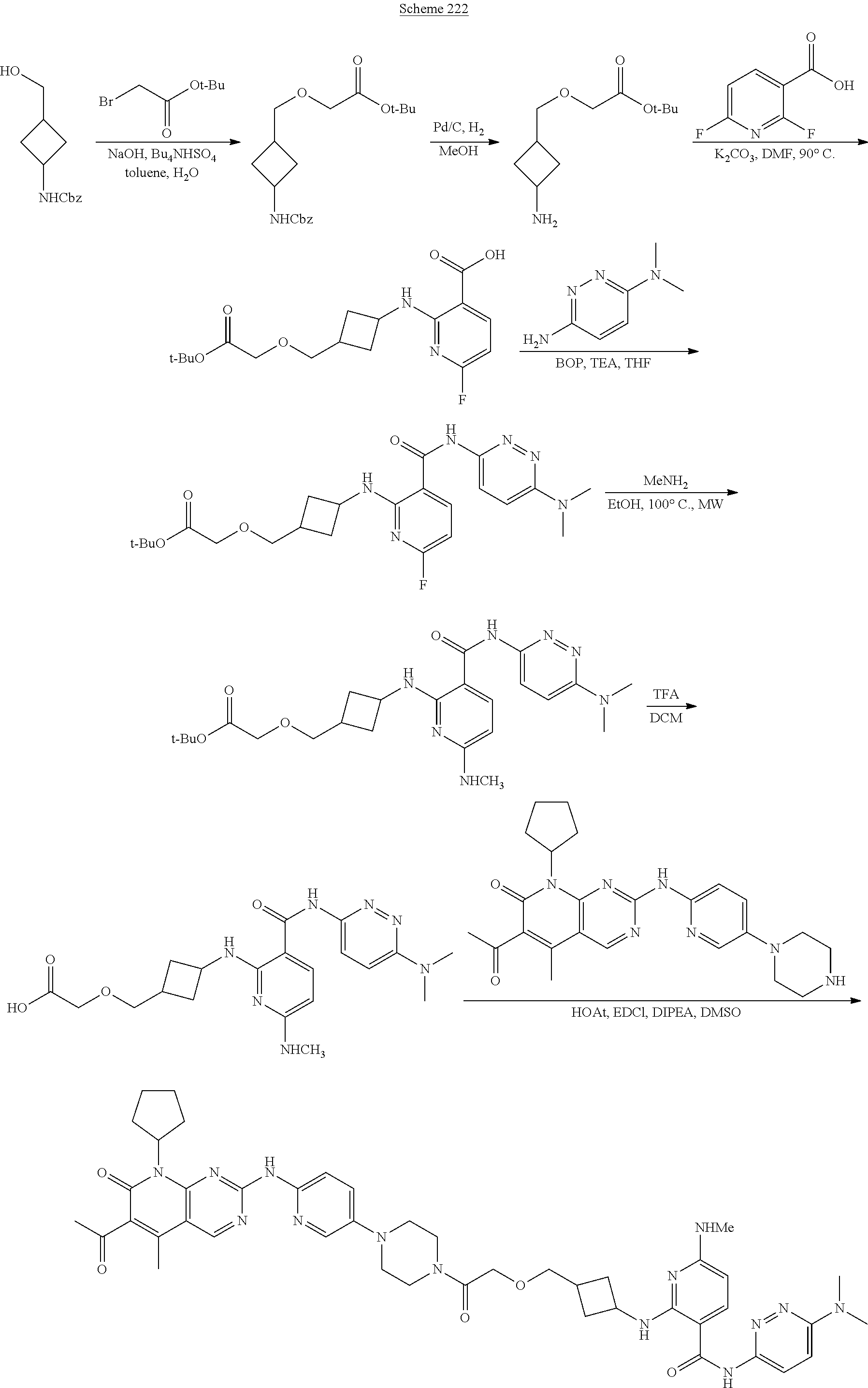

### Step 1. Synthesis of tert-butyl 2-((3-(((benzyloxy)carbonyl)amino)cyclobutyl)methoxy)acetate

To a solution of benzyl (3-(hydroxymethyl)cyclobutyl)carbamate (500 mg, 2.1 mmol), tetrabutylammonium hydrogen sulfate (577 mg, 1.7 mmol) and NaOH (3.7 g, 92.6 mmol) in H₂O (5 mL) and toluene (15 mL) was added tert-butyl 2-bromoacetate (497 mg, 2.6 mmol). The reaction mixture was stirred at rt for 12 h. The mixture was quenched with H₂O (20 mL) and extracted with dichloromethane (40 mL×3). The combined organic phase was washed with brine, dried with anhydrous Na₂SO₄, filtered and concentrated. The residue was purified by silica gel chromatography to give the desired product (300 mg, 40% yield) as a colorless oil. MS (ESI) m/z=350.4 [M+H]⁺.

### Step 2. Synthesis of tert-butyl 2-((3-aminocyclobutyl)methoxy)acetate

To a solution tert-butyl 2-((3-(((benzyloxy)carbonyl)amino)cyclobutyl)methoxy)acetate (300 mg, 0.86 mmol) in methanol (10 mL) was added 10% Pd/C (104 mg). The reaction mixture was purged three times with hydrogen and stirred under hydrogen atmosphere for 12 h. The mixture was filtered through Celite and the filtrate was concentrated under reduced pressure to provide the desired product (180 mg, 97% yield) as a yellow oil. This compound was used directly in the next step without further purification. MS (ESI) m/z=216.5 [M+H]⁺.

The remaining steps were performed following the standard procedures for preparing CPD-145 to provide the desired product (21 mg, 3% yield over 5 steps) as a yellow solid. MS (ESI) m/z=859.9 [M+H]⁺.

### Example 223. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-(methylamino)nicotinamide (CPD-165)

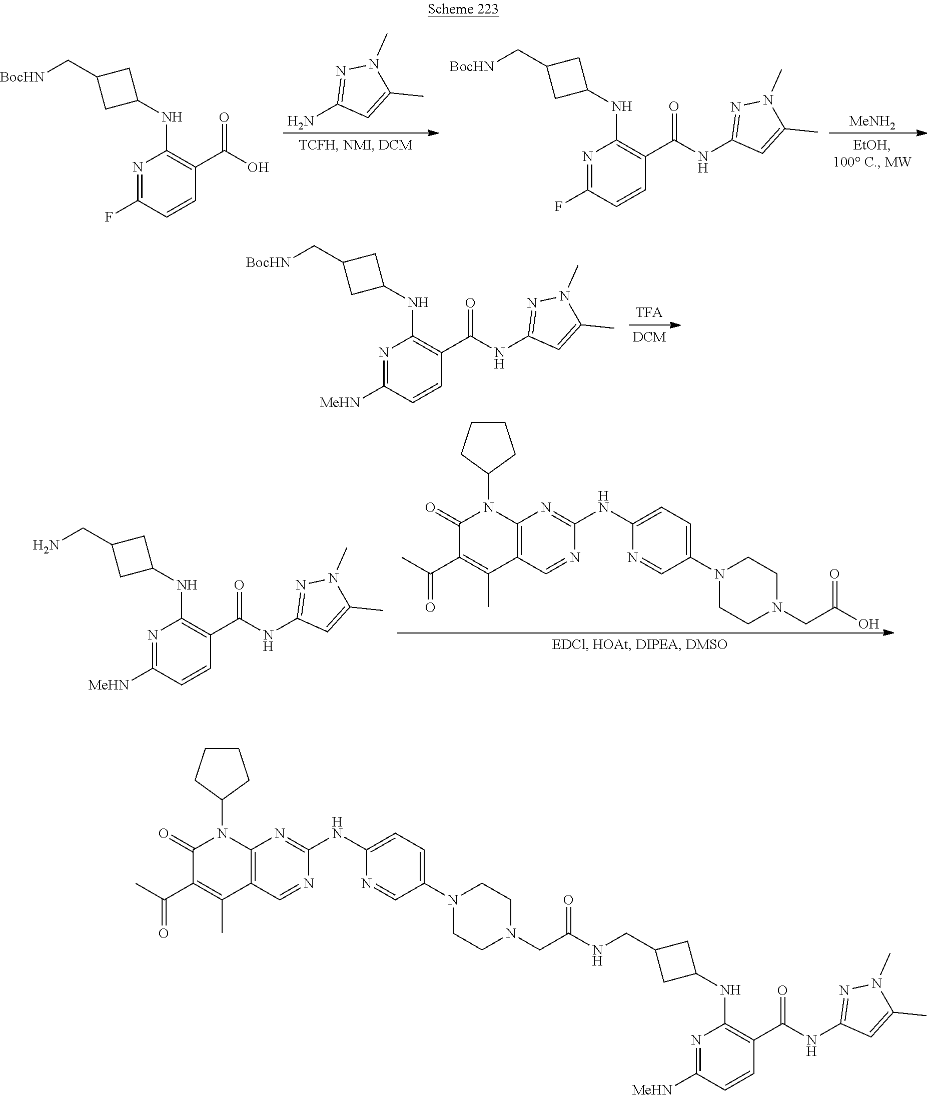

CPD-165 was synthesized following the standard procedures for preparing CPD-144 (4.0 mg, 17% yield over 4 steps) as a yellow solid. MS (ESI) m/z=831.8 [M+H]⁺.

### Example 224. 4-(6-((6-acetyl-8-cyclopentyl-5-methyl-7-oxo-7,8-dihydropyrido[2,3-d]pyrimidin-2-yl)amino)pyridin-3-yl)-N-((3-((3-((6-(dimethylamino)pyridazin-3-yl)carbamoyl)-6-(methylamino)pyridin-2-yl)amino)cyclobutyl)methyl)piperazine-1-carboxamide (CPD-166)

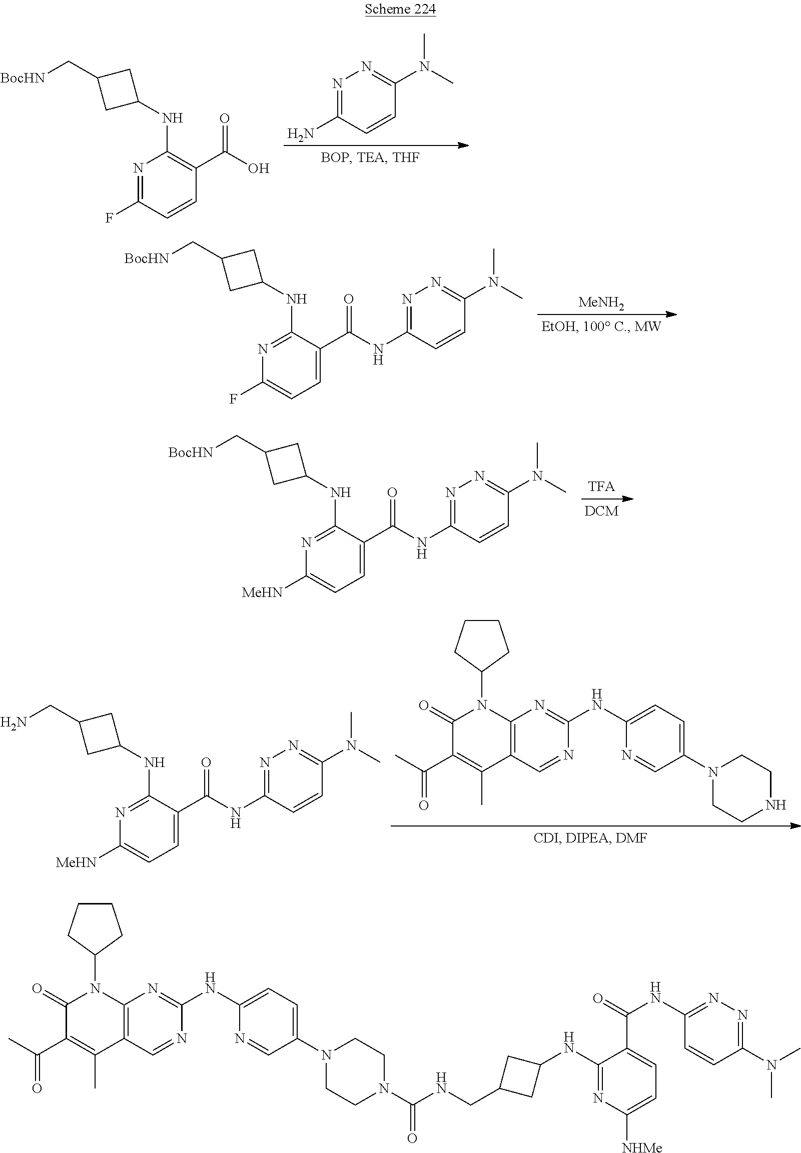

CPD-166 was synthesized following the standard procedures for preparing CPD-145 and CPD-151 (6.4 mg, 24% yield over 4 steps) as a yellow solid. MS (ESI) m/z=844.8 [M+H]⁺.

### Example 225. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)methyl)cyclobutyl)amino)-N-(6-(dimethylamino)pyridazin-3-yl)-6-(methylamino)nicotinamide (CPD-167)

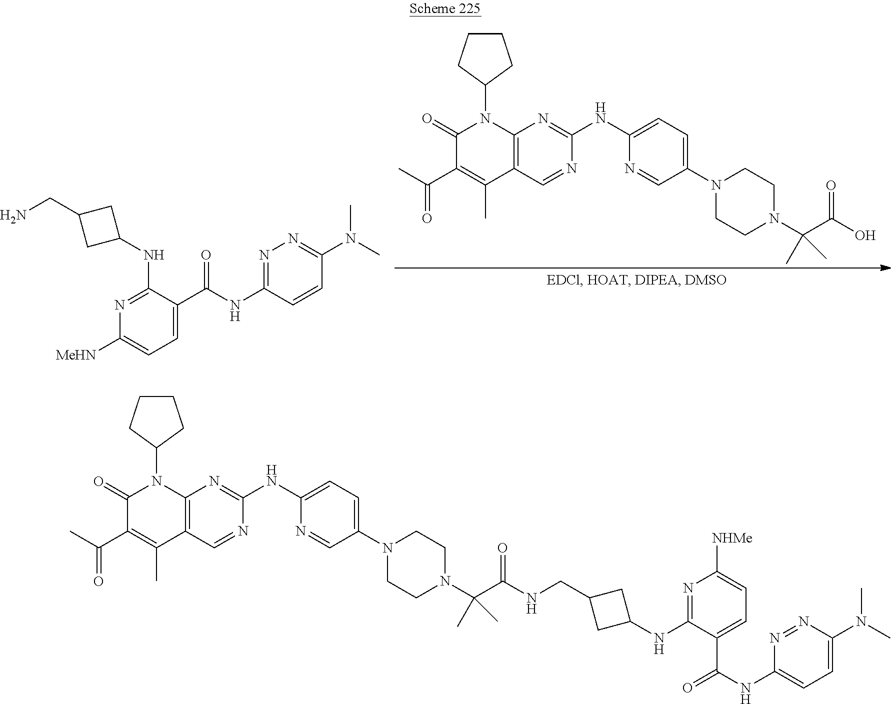

CPD-167 was synthesized following the standard procedure for preparing CPD-143 (4.6 mg, 18% yield) as a yellow solid. MS (ESI) m/z=886.7 [M+H]⁺.

### Example 226. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)cyclobutyl)amino)-N-(6-(dimethylamino)pyridazin-3-yl)-6-(methylamino)nicotinamide (CPD-168)

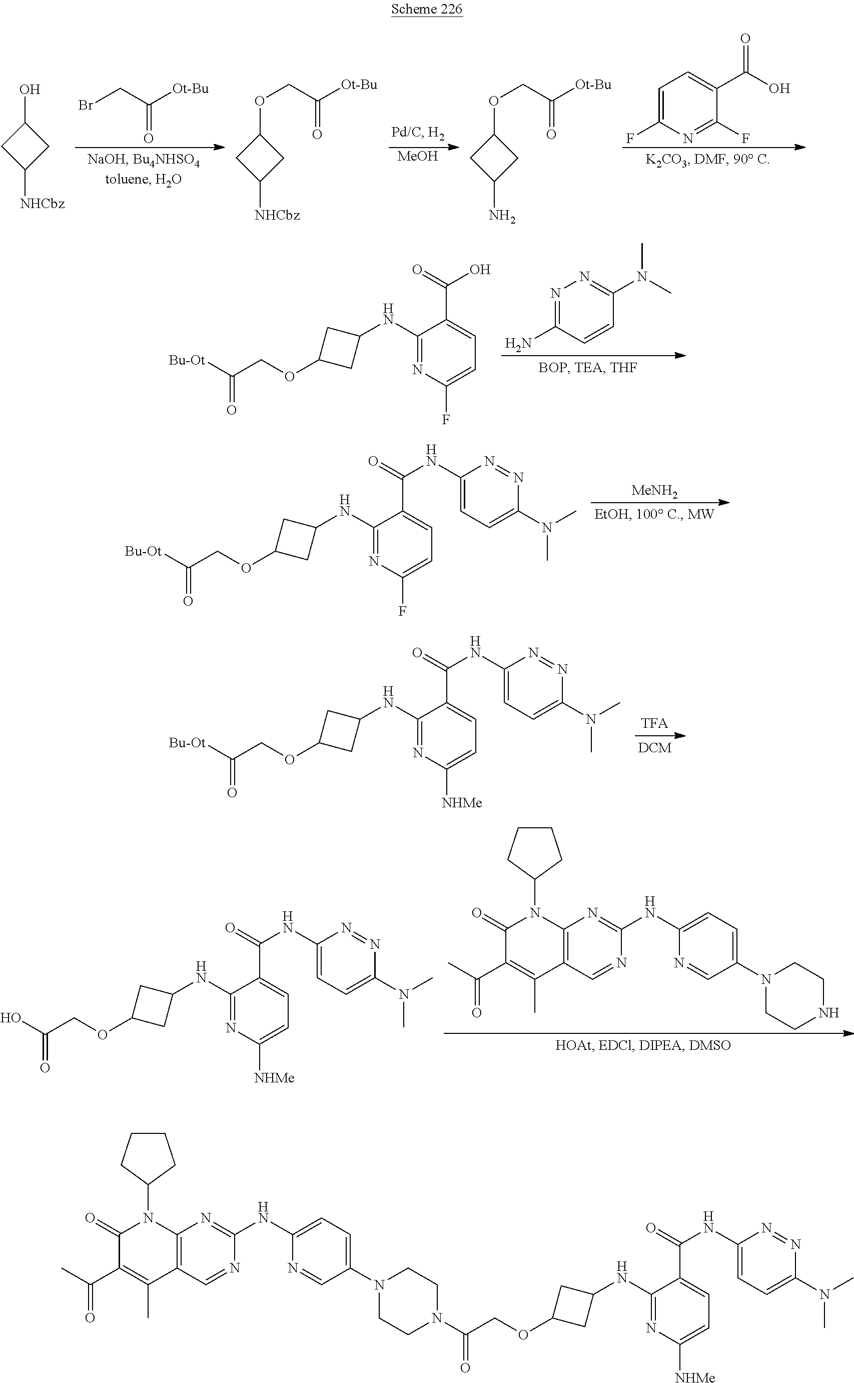

CPD-168 was synthesized following the standard procedures for preparing CPD-164 (42 mg, 2% yield over 7 steps) as a yellow solid. MS (ESI) m/z=845.8 [M+H]⁺.

### Example 227. 4-(6-((6-acetyl-8-cyclopentyl-5-methyl-7-oxo-7,8-dihydropyrido[2,3-d]pyrimidin-2-yl)amino)pyridin-3-yl)-N-((trans-4-(((3-((6-(dimethylamino)pyridazin-3-yl)carbamoyl)-6-(methylamino)pyridin-2-yl)amino)methyl)cyclohexyl)menthyl)piperazine-1-carboxamide (CPD-169)

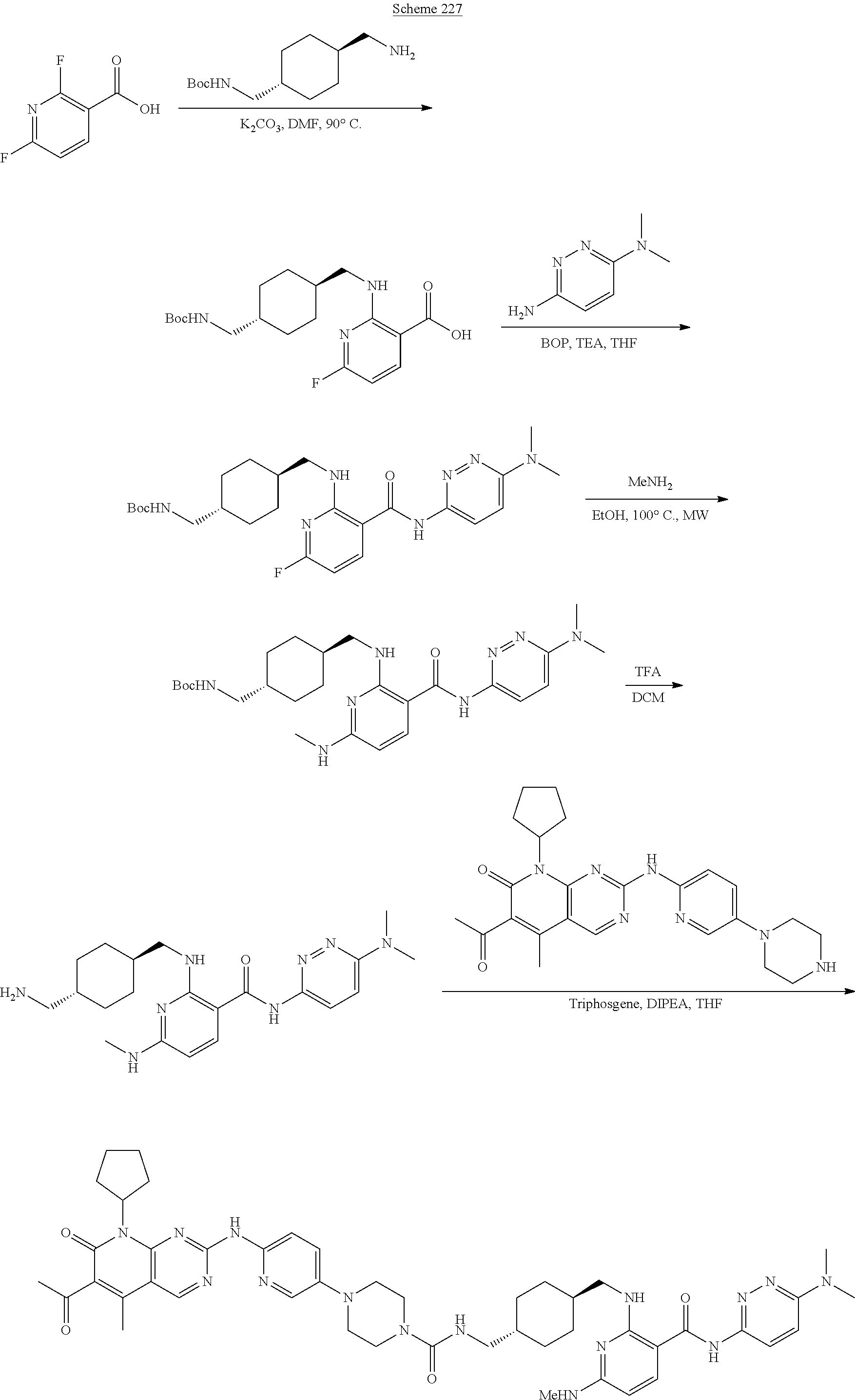

### Step 1 to step 4. Synthesis of 2-(((trans-4-(aminomethyl)cyclohexyl)methyl)amino)-N-(6-(dimethyl amino)pyridazin-3-yl)-6-(methylamino)nicotinamide

These steps were performed following the standard procedures for preparing CDP-145 to provide the desired product (35 mg, 18% yield over 4 steps) as a pale-yellow solid. MS (ESI) m/z=413.6 [M+H]⁺.

### Step 5. Synthesis of 4-(6-((6-acetyl-8-cyclopentyl-5-methyl-7-oxo-7,8-dihydropyrido[2,3-d]pyrimidin-2-yl)amino)pyridin-3-yl)-N-((trans-4-(((3-((6-(dimethylamino)pyridazin-3-yl)carbamoyl)-6-(methylamino)pyridin-2-yl)amino)methyl)cyclohexyl)methyl)piperazine-1-carboxamide

A mixture of 6-acetyl-8-cyclopentyl-5-methyl-2-[(5-piperazin-1-yl-2-pyridyl)amino]pyrido[2,3-d]pyrimidin-7-one (13.1 mg, 29.1 μmol), DIPEA (12.5 mg, 97.0 μmol) and triphosgene (3.6 mg, 12.1 μmol) in THF (1 mL) was stirred at 0 [symbol] for 1 h, followed by adding 2-(((trans-4-(aminomethyl)cyclohexyl)methyl)amino)-N-(6-(dimethylamino)pyridazin-3-yl)-6-(methylamino)nicotinamide (10 mg, 24.2 μmol). The resulting mixture was stirred at 50 [symbol] for 2 h. After cooling down to 0 T, the reaction mixture was diluted with dichloromethane (10 mL) and aq. HCl solution (1 M, 2 mL). The organic layer was separated and concentrated under reduced pressure. The residue was purified by silica gel chromatography and prep-HPLC to give the desired product (1.3 mg, 6% yield) as a yellow solid. MS (ESI) m/z=886.7 [M+H]⁺.

### Example 228. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(methylamino)-N-(6-methylpyridin-2-yl)nicotinamide (CPD-170)

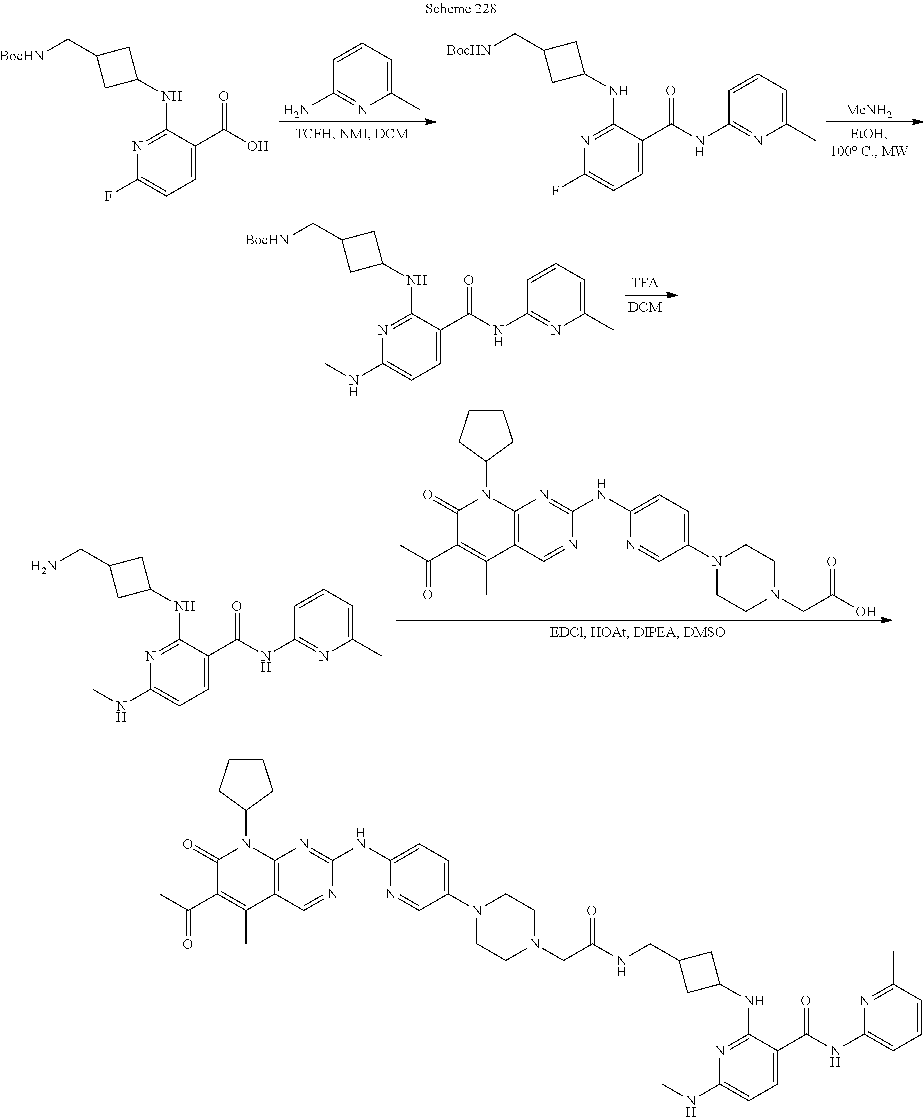

CPD-170 was synthesized following the standard procedures for preparing CPD-144 (13 mg, 21% yield over 4 steps) as a yellow solid. MS (ESI) m/z=828.8 [M+H]⁺.

### Example 229. 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-6-(methylamino)-N-(6-methylpyridin-2-yl)nicotinamide (CPD-171)

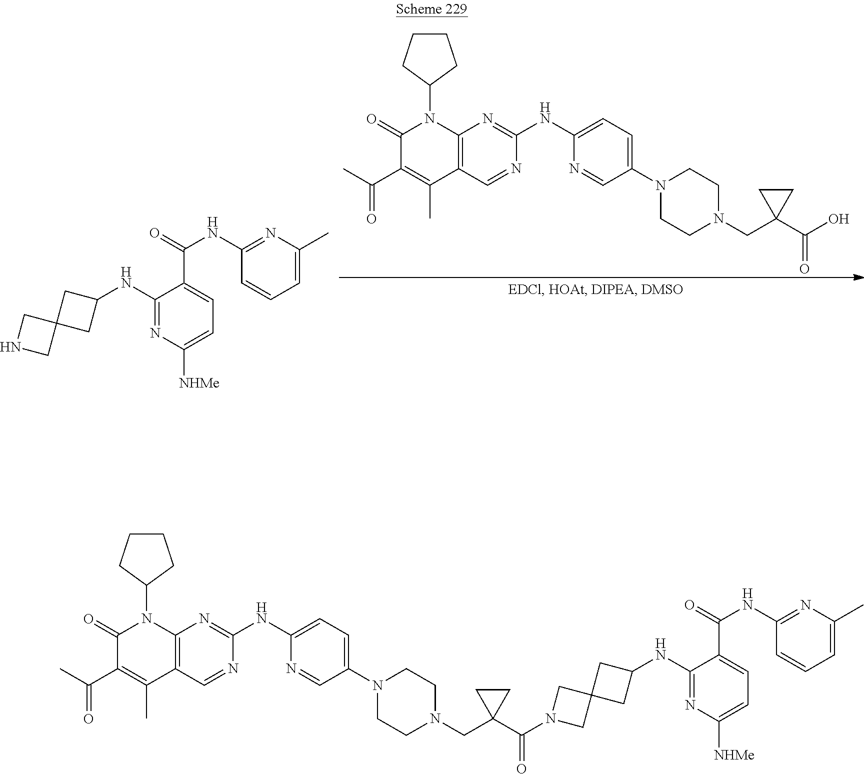

CPD-171 was synthesized following the standard procedure for preparing CPD-143 (2.3 mg, 29% yield) as a yellow solid. MS (ESI) m/z=880.8 [M+H]⁺.

### Example 230. 2-((3-((1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carboxamido)methyl)cyclobutyl)amino)-N-(6-(dimethylamino)pyridazin-3-yl)-6-(methylamino)nicotinamide (CPD-172)

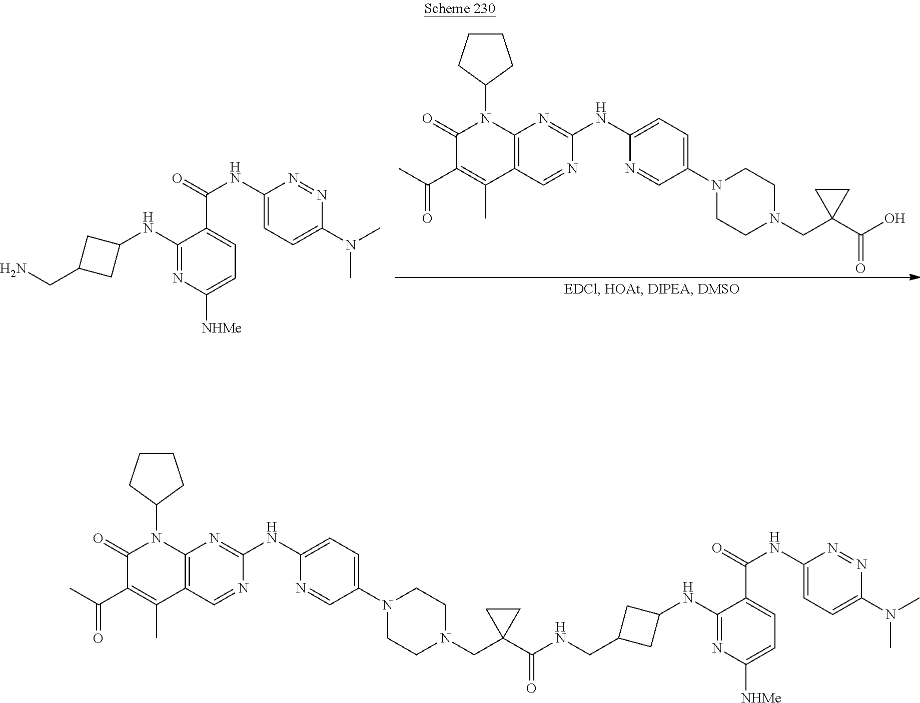 [SMILES: CNc1ccc(C(=O)Nc2ccc(N(C)C)nn2)c(NC2CC(CN)C2)n1 ; CC(=O)c1c(C)c2cnc(Nc3ccc(N4CCN(CC5(C(=O)O)CC5)CC4)cn3)nc2n(C2CCCC2)c1=O ; CNc1ccc(C(=O)Nc2ccc(N(C)C)nn2)c(NC2CC(CNC(=O)C3(CN4CCN(c5ccc(Nc6ncc7c(C)c(C(C)=O)c(=O)n(C8CCCC8)c7n6)nc5)CC4)CC3)C2)n1]

CPD-172 was synthesized following the standard procedure for preparing CPD-143 (2.2 mg, 29% yield) as a yellow solid. MS (ESI) m/z=898.8 [M+H]⁺.

### Example 231. 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(6-cyclopropylpyridin-2-yl)-6-(methylamino)nicotinamide (CPD-173)

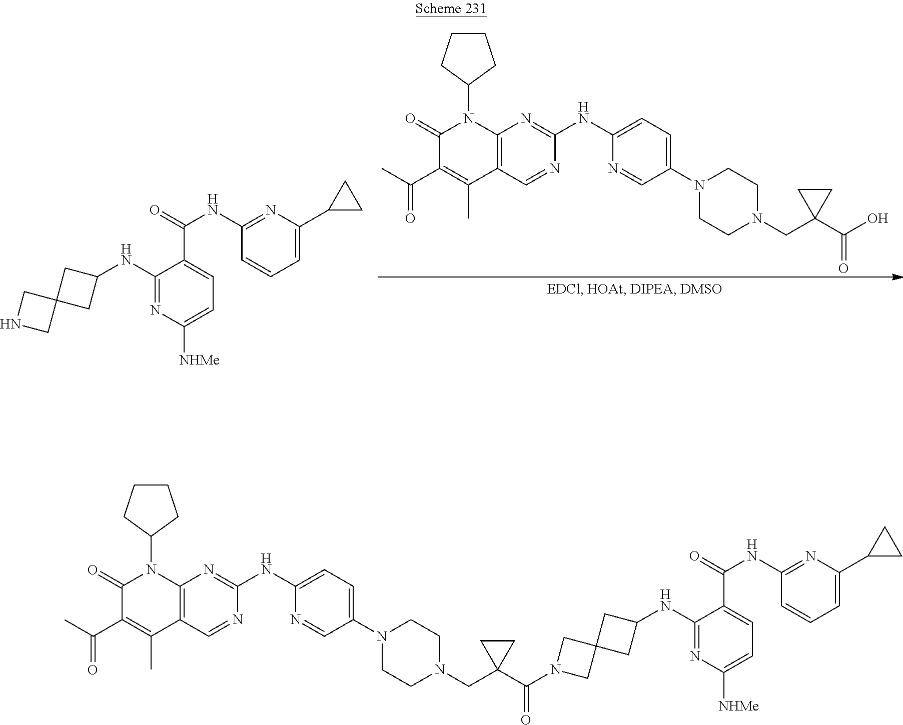

CPD-173 was synthesized following the standard procedure for preparing CPD-143 (2.4 mg, 2700 yield) as a yellow solid. MS (ESI) m/z=906.8 [M+H]⁺.

### Example 232. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)spiro[3.3]heptan-2-yl)amino)-N-(6-(dimethylamino)pyridazin-3-yl)-6-(methylamino)nicotinamide (CPD-174)

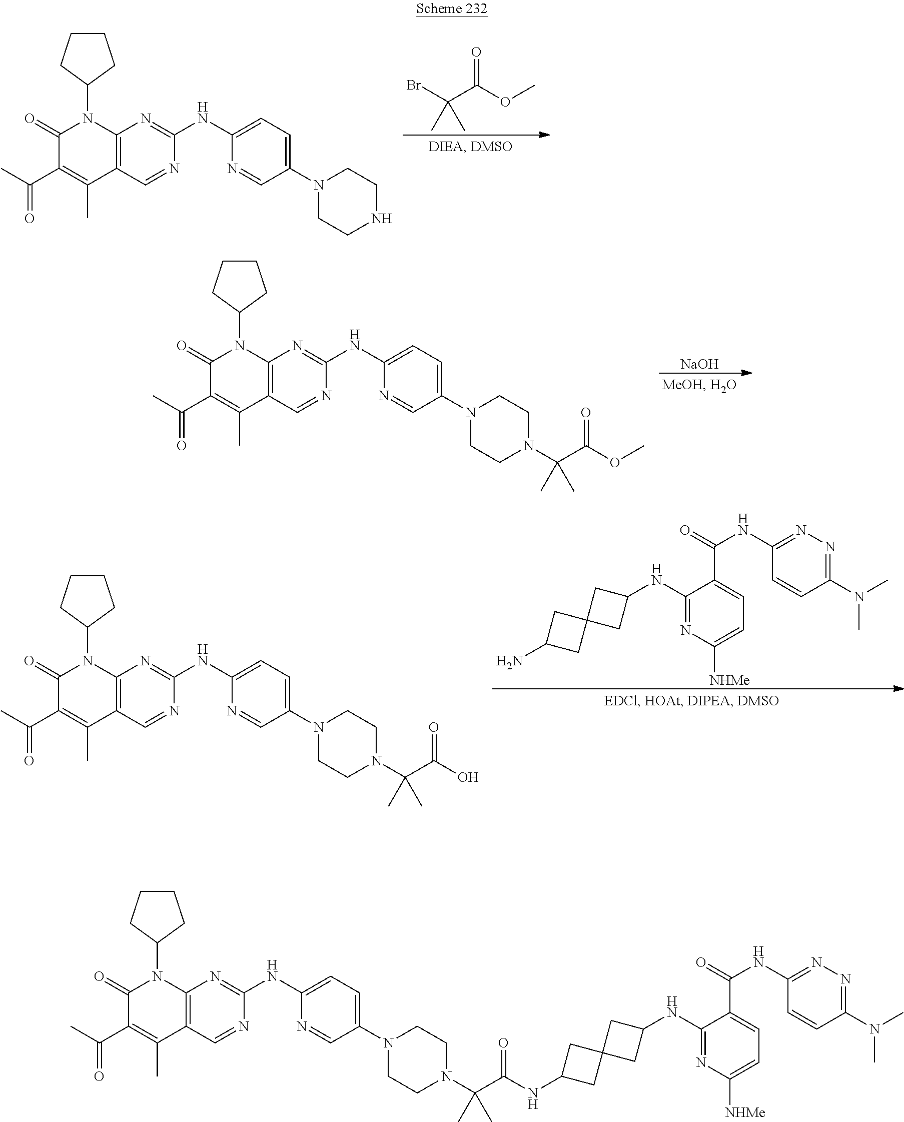

### Step 1. Synthesis of methyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoate

To a solution of 6-acetyl-8-cyclopentyl-5-methyl-2-((5-(piperazin-1-yl)pyridin-2-yl)amino)pyrido[2,3-d]pyrimidin-7(8H)-one (140 mg, 0.313 mmol) and methyl 2-bromo-2-methylpropanoate (169.9 mg, 0.938 mmol) in DMSO (6 mL) was added DIEA (404.3 mg, 3.13 mmol). The reaction mixture was stirred at 90° C. for 16 h. After cooling down to rt, the mixture was purified by reverse-phase chromatography to give the desired product (100 mg, 58% yield) as a light-yellow solid. MS (ESI) m/z=548.3 [M+H]⁺.

### Step 2. Synthesis of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoic acid

To a solution of methyl 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoate (100 mg, 182.60 μmol) in MeOH (5 mL) were added NaOH (73.0 mg, 1.83 mmol) and H₂O (1 ml). The mixture was stirred at 80° C. for 16 h, before it was purified by reverse-phase chromatography to give the desired product (70 mg, 72% yield) as a yellow solid. MS (ESI) m/z=534.3 [M+H]⁺.

### Step 3. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)spiro[3.3]heptan-2-yl)amino)-N-(6-(dimethylamino)pyridazin-3-yl)-6-(methylamino)nicotinamide

The title compound was synthesized following the standard procedure for preparing CPD-143 (2.2 mg, 16% yield) as a light-yellow solid. MS (ESI) m/z=912.8 [M+H]⁺.

### Example 233. 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanoyl)-2-azaspiro[3.3]heptan-6-yl)amino)-N-(6-cyclopropylpyridin-2-yl)-6-(methylamino)nicotinamide (CPD-175)

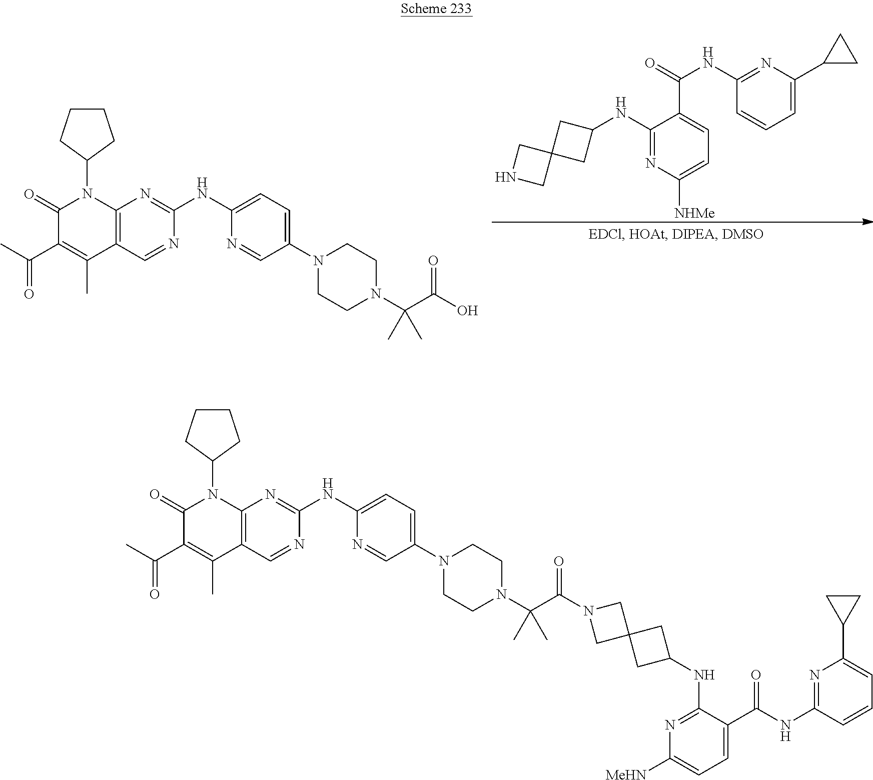 [SMILES: CC(=O)c1c(C)c2cnc(Nc3ccc(N4CCN(C(C)(C)C(=O)O)CC4)cn3)nc2n(C2CCCC2)c1=O ; CNc1ccc(C(=O)Nc2cccc(C3CC3)n2)c(NC2CC3(CNC3)C2)n1 ; CNc1ccc(C(=O)Nc2cccc(C3CC3)n2)c(NC2CC3(C2)CN(C(=O)C(C)(C)N2CCN(c4ccc(Nc5ncc6c(C)c(C(C)=O)c(=O)n(C7CCCC7)c6n5)nc4)CC2)C3)n1]

CPD-175 was synthesized following the standard procedure for preparing CPD-143 (4.0 mg, 19% yield) as a light-yellow solid. MS (ESI) m/z=894.7 [M+H]⁺.

### Example 234. 2-((2-(2-(4-(6-((6-acetyl-8-cyclopentyl-5-methyl-7-oxo-7,8-dihydropyrido[2,3-di]pyrimidin-2-yl)amino)pyridin-3-yl)piperazin-1-yl)-2-methylpropanoyl)-2-azaspiro[3.3]heptan-6-yl)amino)-6-(methylamino)-N-(6-methylpyridin-2-yl)nicotinamide (CPD-176)

### Scheme 234

CPD-176 was synthesized following the standard procedure for preparing CPD-143 (2.1 mg, 17% yield) as a light-yellow solid. MS (ESI) m/z=868.8 [M+H]⁺.

### Example 235. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-(dimethylamino)pyridin-2-yl)-6-(methylamino)nicotinamide (CPD-177)

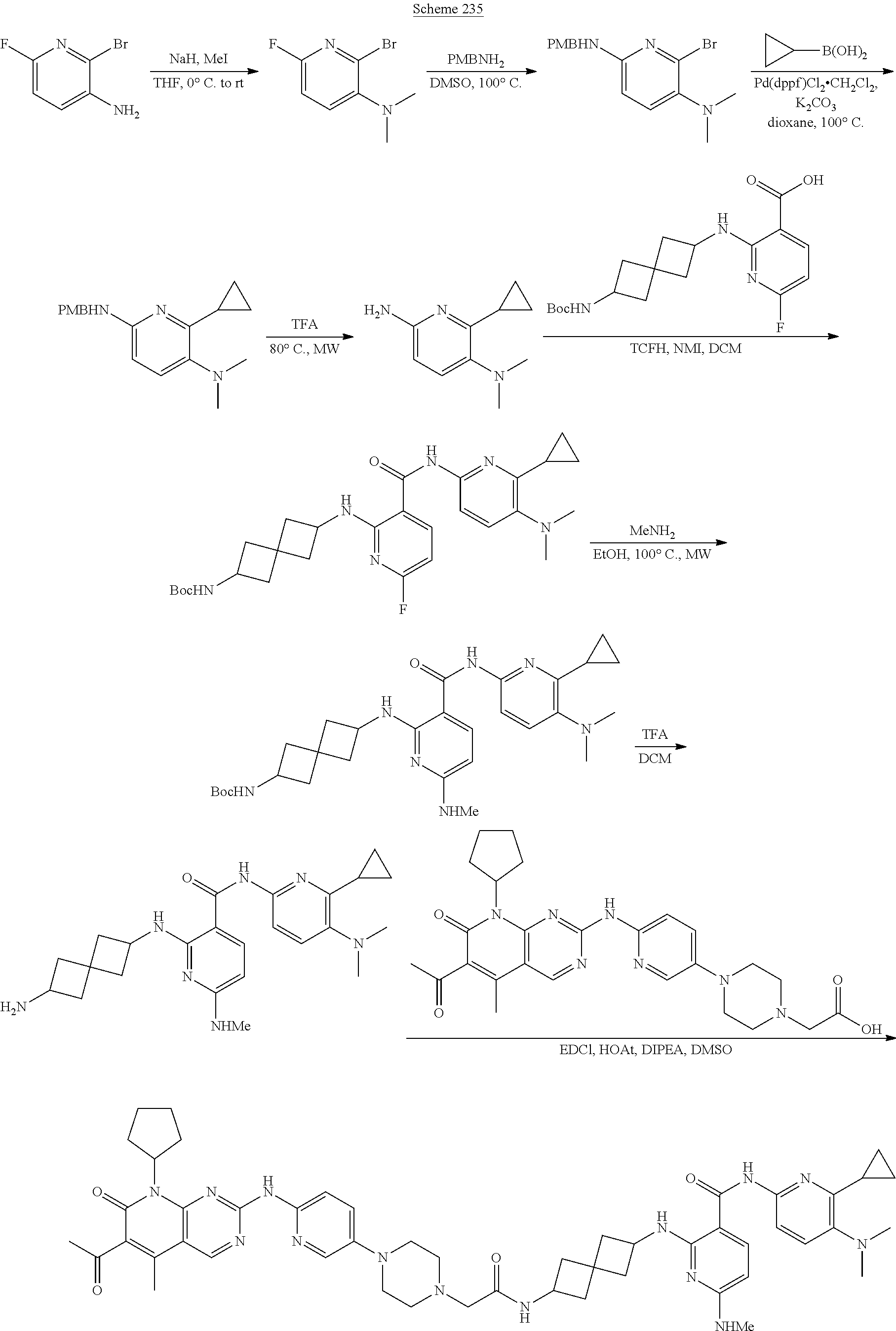

### Step 1. Synthesis of 2-bromo-6-fluoro-N,N-dimethylpyridin-3-amine

To a solution of 2-bromo-6-fluoro-pyridin-3-amine (2.00 g, 10.5 mmol) in THF (20 mL) was added NaH (1.30 g, 32.5 mmol, 60% in mineral oil) at 0 [symbol] . After the reaction mixture was stirred at 0 [symbol] or 0.5 h, it was added MeI (9.12 g, 64.3 mmol) at 0 [symbol] . The reaction mixture was stirred at rt for 12 h. The reaction mixture was quenched with aq. NH₄Cl solution (10 mL) and extracted with ethyl acetate (20 mL×3).

The combined organic layers were washed with brine (40 mL), dried over Na₂SO₄, filtered and concentrated. The residue was purified by silica gel column chromatography (petroleum ether/ethyl acetate=10:1 to 7:1) to provide the desired product (1.20 g, 52% yield) as a colorless oil. ¹HNMR (400 MHz, CDCl₃) δ 7.49-7.44 (m, 1H), 6.89-6.84 (m, 1H), 2.79 (s, 6H). MS (ESI) m/z=221.1 [M+H]⁺.

### Step 2. Synthesis of 6-bromo-N²-(4-methoxybenzyl)-N⁵,N⁵-dimethylpyridine-2,5-diamine

To a solution of 2-bromo-6-fluoro-N,N-dimethylpyridin-3-amine (1.00 g, 4.57 mmol) in DMSO (3 mL) was added PMBNH₂ (3.13 g, 22.8 mmol) at rt. The reaction was stirred at 100 [symbol] for 12 h. The reaction mixture was diluted with H₂O (10 mL) and extracted with ethyl acetate (10 mL×3). The combined organic layers were dried over Na₂SO₄, filtered, and concentrated under reduced pressure to give a residue. The residue was purified by silica gel column chromatography (petroleum ether/ethyl acetate=5:1) to provide the desired product (0.90 g, 59% yield) as a yellow oil. ¹HNMR (400 MHz, CDCl₃) δ 7.33-7.26 (m, 3H), 6.95-6.86 (m, 2H), 6.32 (d, J=8.8 Hz, 1H), 4.89 (s, 1H), 4.40 (d, J=5.2 Hz, 2H), 3.84 (s, 3H), 2.73 (s, 6H). MS (ESI) m/z=366.3 [M+H]⁺.

### Step 3. Synthesis of 6-cyclopropyl-N²-(4-methoxybenzyl)-N⁵,N⁵-dimethylpyridine-2,5-diamine

To a mixture of 6-bromo-A¹-[(4-methoxyphenyl)methyl]-N⁵,N⁵-dimethyl-pyridine-2,5-diamine (0.90 g, 2.68 mmol) in dioxane (20 mL) were added cyclopropylboronic acid (1.15 g, 13.4 mmol), K₂CO₃ (1.13 g, 8.14 mmol) and Pd(dppf)Cl₂·CH₂Cl₂ (220 mg, 269 μmol) at rt. The reaction mixture was stirred at 100° C. for 12 h. The reaction mixture was poured into H₂O (20 mL) and extracted with ethyl acetate (20 mL×3). The combined organic layers were washed with brine (40 mL), dried over Na₂SO₄, filtered and concentrated. The residue was purified by silica gel column chromatography (petroleum ether/ethyl acetate=5:1) to provide the desired product (260 mg, 33% yield) as a yellow oil. ¹HNMR (400 MHz, CDCl₃) δ 7.26 (d, J=7.2 Hz, 2H), 7.16 (d, J=8.4 Hz, 1H), 6.85 (d, J=8.4 Hz, 2H), 6.11 (d, J=8.4 Hz, 1H), 4.46 (s, 1H), 4.33 (d, J=4.4 Hz, 2H), 3.79 (s, 3H), 2.66 (s, 6H), 2.52-2.40 (m, 1H), 1.01 (s, 2H), 0.90-0.78 (m, 2H). MS (ESI) m/z=298.2 [M+H]⁺.

### Step 4. Synthesis of 6-cyclopropyl-N⁵,N⁵-dimethylpyridine-2,5-diamine

A solution of 6-cyclopropyl-N²-[(4-methoxyphenyl)methyl]-N⁵,N⁵-dimethyl-pyridine-2,5-diamine (260 mg, 874 μmol) in TFA (1 mL) was stirred at 80° C. under microwave irradiation for 2 h. After cooling down to rt, the reaction mixture was adjusted pH to 9 with aq. NaOH solution (2 N). The aqueous layer was extracted with ethyl acetate (20 mL×3). The combined organic layers were washed with brine (50 mL), dried over Na₂SO₄, and filtered. The filtrate was concentrated and purified by prep-TLC (dichloromethane/methanol=10:1) to provide the desired product (80.5 mg, 50% yield) as a yellow oil. ¹HNMR (400 MHz, CDCl₃) δ 7.18 (d, J=8.8 Hz, 1H), 6.25 (d, J=8.4 Hz, 1H), 4.11 (s, 2H), 2.68 (s, 6H), 2.51-2.44 (m, 1H), 1.05-0.99 (m, 2H), 0.92-0.87 (m, 2H). MS (ESI) m/z=178.4 [M+H]⁺.

The remaining steps were performed following the standard procedures for preparing CPD-144 to provide the desired product (2 mg, 7% yield over 4 steps) as a yellow solid. MS (ESI) m/z=923.7 [M+H]⁺.

### Example 236. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)cyclobutyl)amino)-N-(6-(dimethylamino)pyridazin-3-yl)-6-(methylamino)nicotinamide (CPD-178)

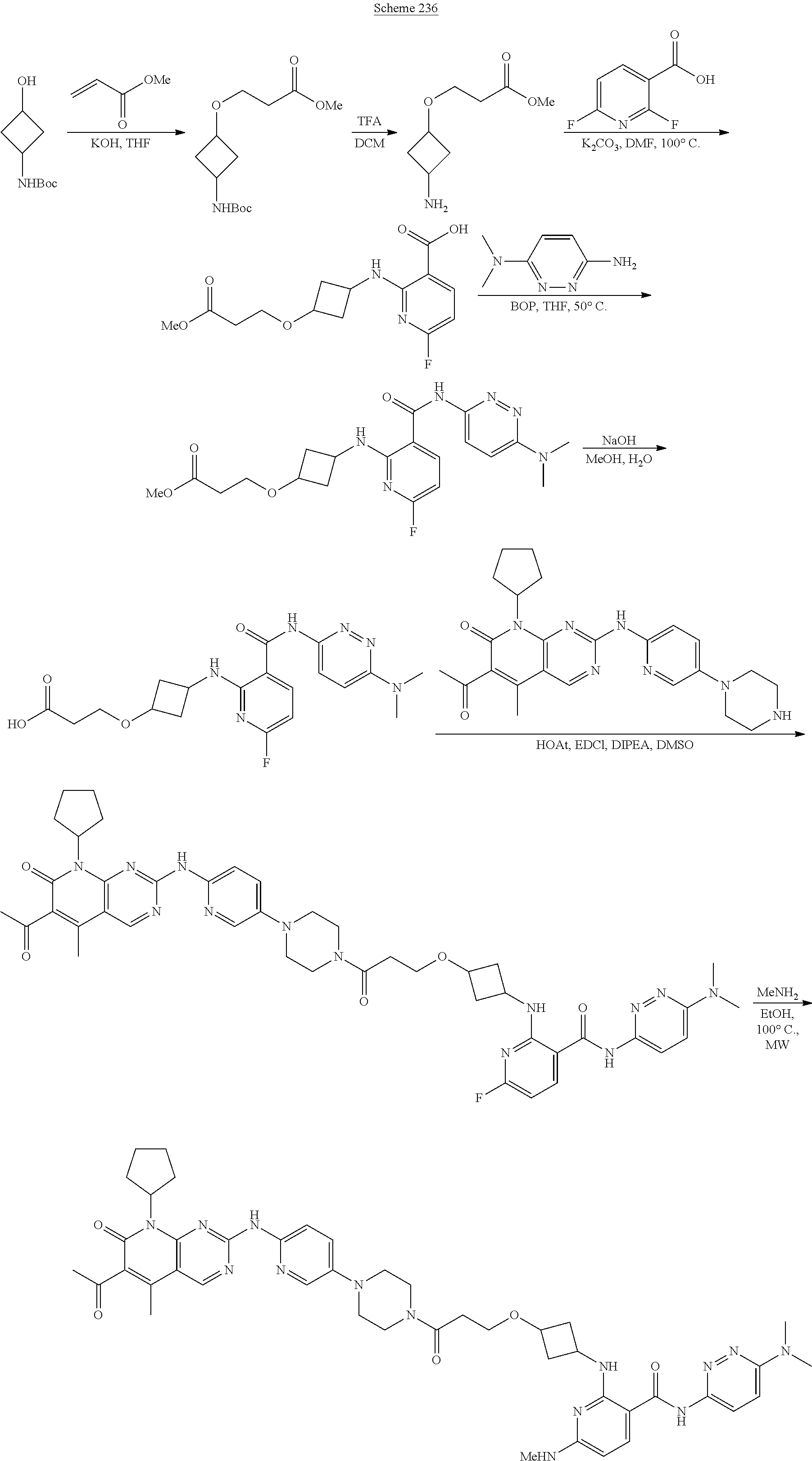

### Step 1. Synthesis of methyl 3-(3-((tert-butoxycarbonyl)amino)cyclobutoxy)propanoate

To a solution of benzyl (3-hydroxycyclobutyl)carbamate (1 g, 5.3 mmol) and methyl acrylate (920 mg, 10.7 mmol) in THF (20 mL) was added KOH (30 mg, 0.53 mmol). The reaction was stirred at rt for 12 h. The mixture was quenched with H₂O (20 mL) and extracted with dichloromethane (20 mL×3). The organic phase was washed with brine, dried with anhydrous Na₂SO₄, filtered and concentrated. The residue was purified by silica gel chromatography to give the desired product (1 g, 68% yield) as a colorless oil. MS (ESI) m/z=274.3 [M+H]⁺.

### Step 2. Synthesis of methyl 3-(3-aminocyclobutoxy)propanoate

To a solution of methyl 3-(3-((tert-butoxycarbonyl)amino)cyclobutoxy)propanoate (1 g, 3.7 mmol) in dichloromethane (10 mL) was added TFA (0.5 mL). The mixture was stirred at rt for 1 h. The resulting mixture was concentrated to give the crude product (600 mg, 95% yield) as a yellow oil. MS (ESI) m/z=174.2 [M+H]⁺.

### Step 3. Synthesis of 6-fluoro-2-((3-(3-methoxy-3-oxopropoxy)cyclobutyl)amino)nicotinic acid

To a solution of methyl 3-(3-aminocyclobutoxy)propanoate (600 mg, 3.5 mmol) and 2,6-difluoronicotinic acid (661 mg, 4.2 mmol) in DMF (5 mL) was added K₂CO₃ (957 mg, 6.9 mmol). The reaction was stirred at 100° C. for 12 h. The mixture was quenched with H₂O (20 mL) and extracted with dichloromethane (20 mL×3). The organic phase was washed with brine, dried with anhydrous Na₂SO₄, filtered and concentrated. The residue was purified by silica gel chromatography to give the desired product (400 mg, 39% yield) as a yellow oil. MS (ESI) m/z=313.4 [M+H]⁺.

### Step 4. Synthesis of methyl 3-(3-((3-((6-(dimethylamino)pyridazin-3-yl)carbamoyl)-6-fluoropyridin-2-yl)amino)cyclobutoxy)propanoate

To a solution of 6-fluoro-2-((3-(3-methoxy-3-oxopropoxy)cyclobutyl)amino)nicotinic acid (400 mg, 1.3 mmol), N³,N³-dimethylpyridazine-3,6-diamine (177 mg, 1.3 mmol) and BOP (523 mg, 2.6 mmol) in THF (10 mL) was added DIPEA (0.5 mL). The reaction was stirred at 50 [symbol] for 12 h. The mixture was quenched with H₂O (20 mL) and extracted with ethyl acetate (20 mL×3). The organic phase was washed with brine, dried with anhydrous Na₂SO₄, filtered and concentrated. The residue was purified by silica gel chromatography to give the desired product (320 mg, 58% yield) as a yellow oil. MS (ESI) m/z=433.6 [M+H]⁺.

### Step 5. Synthesis of 3-(3-((3-((6-(dimethylamino)pyridazin-3-yl)carbamoyl)-6-fluoropyridin-2-yl)amino)cyclobutoxy)propanoic acid

To a solution of methyl 3-(3-((3-((6-(dimethylamino)pyridazin-3-yl)carbamoyl)-6-fluoropyridin-2-yl)amino)cyclobutoxy)propanoate (70 mg, 0.16 mmol) in H₂O (5 mL) and methanol (5 mL) was added NaOH (13 mg, 0.32 mmol). The mixture was stirred at rt for 2 h. The mixture was concentrated under vacuum to obtain the residue. The residue was re-dissolved in H₂O (5 mL). The pH value was adjusted to 4 by adding aq. HCl (4 N). The mixture was extracted with dichloromethane (20 mL×3).

The organic phase was washed with brine, dried over anhydrous Na₂SO₄, filtered. The filtrate was concentrated to give the desired product (50 mg, 74% yield) as a yellow solid. The residue was used directly in the next step without further purification. MS (ESI) m/z=419.4 [M+H]⁺.

### Step 6. Synthesis of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)cyclobutyl)amino)-N-(6-(dimethylamino)pyridazin-3-yl)-6-fluoronicotinamide

To a solution of 3-(3-((3-((6-(dimethylamino)pyridazin-3-yl)carbamoyl)-6-fluoropyridin-2-yl)amino)cyclobutoxy)propanoic acid (50 mg, 0.12 mmol), 6-acetyl-8-cyclopentyl-5-methyl-2-((5-(piperazin-1-yl)pyridin-2-yl)amino)pyrido[2,3-d]pyrimidin-7(8H)-one (64 mg, 0.14 mmol), EDCl (34 mg, 0.18 mmol) and HOAt (24 mg, 0.18 mmol) in DMSO (2 mL) was added DIPEA (0.1 mL). The reaction was stirred at rt for 12 h. The mixture was quenched with H₂O (20 mL) and extracted with dichloromethane (20 mL×3). The organic phase was washed with brine, dried with anhydrous Na₂SO₄, filtered and concentrated. The residue was purified by silica gel chromatography to give the desired product (60 mg, 59% yield) as a yellow solid. MS (ESI) m/z=848.7 [M+H]⁺.

### Step 7. Synthesis of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)cyclobutyl)amino)-N-(6-(dimethylamino)pyridazin-3-yl)-6-(methylamino)nicotinamide

A mixture of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropoxy)cyclobutyl)amino)-N-(6-(dimethylamino)pyridazin-3-yl)-6-fluoronicotinamide (60 mg, 0.07 mmol) and methylamine (30% in EtOH, 0.2 mL) in EtOH (1 mL) was stirred at 100 [symbol] under microwave irradiation for 1 h. After cooling down to rt, the mixture was concentrated and purified by prep-HPLC to give the desired product (24.6 mg, 40% yield) as a yellow solid. MS (ESI) m/z=860.0 [M+H]⁺.

### Example 237. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,2-dimethyl-1H-imidazol-4-yl)-6-(methylamino)nicotinamide (CPD-179)

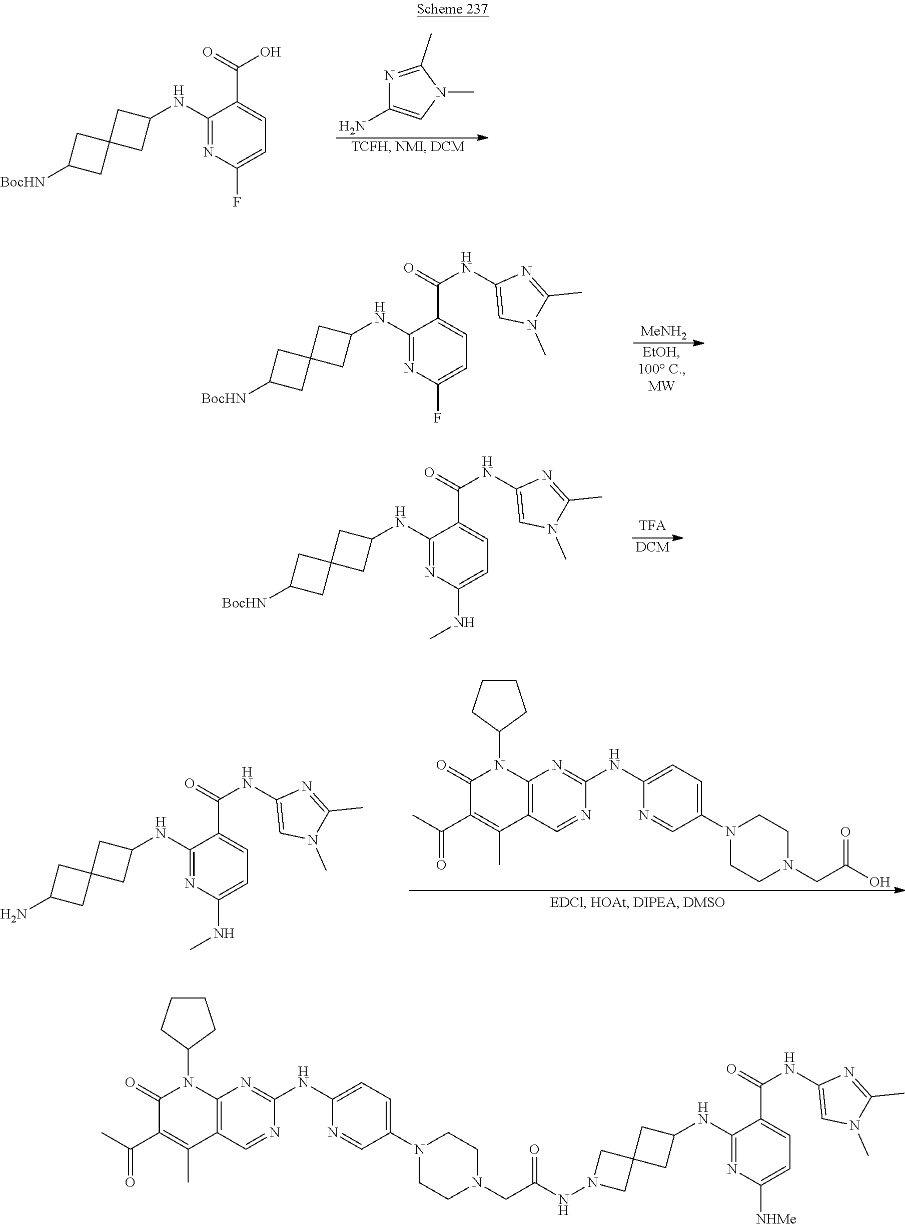

CPD-179 was synthesized following the standard procedures for preparing CPD-144 (6.2 mg, 12% yield over 4 steps) as a yellow solid. MS (ESI) m/z=857.9 [M+H]⁺.

### Example 238. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-aminopyridin-2-yl)-6-(methylamino)nicotinamide (CPD-180)

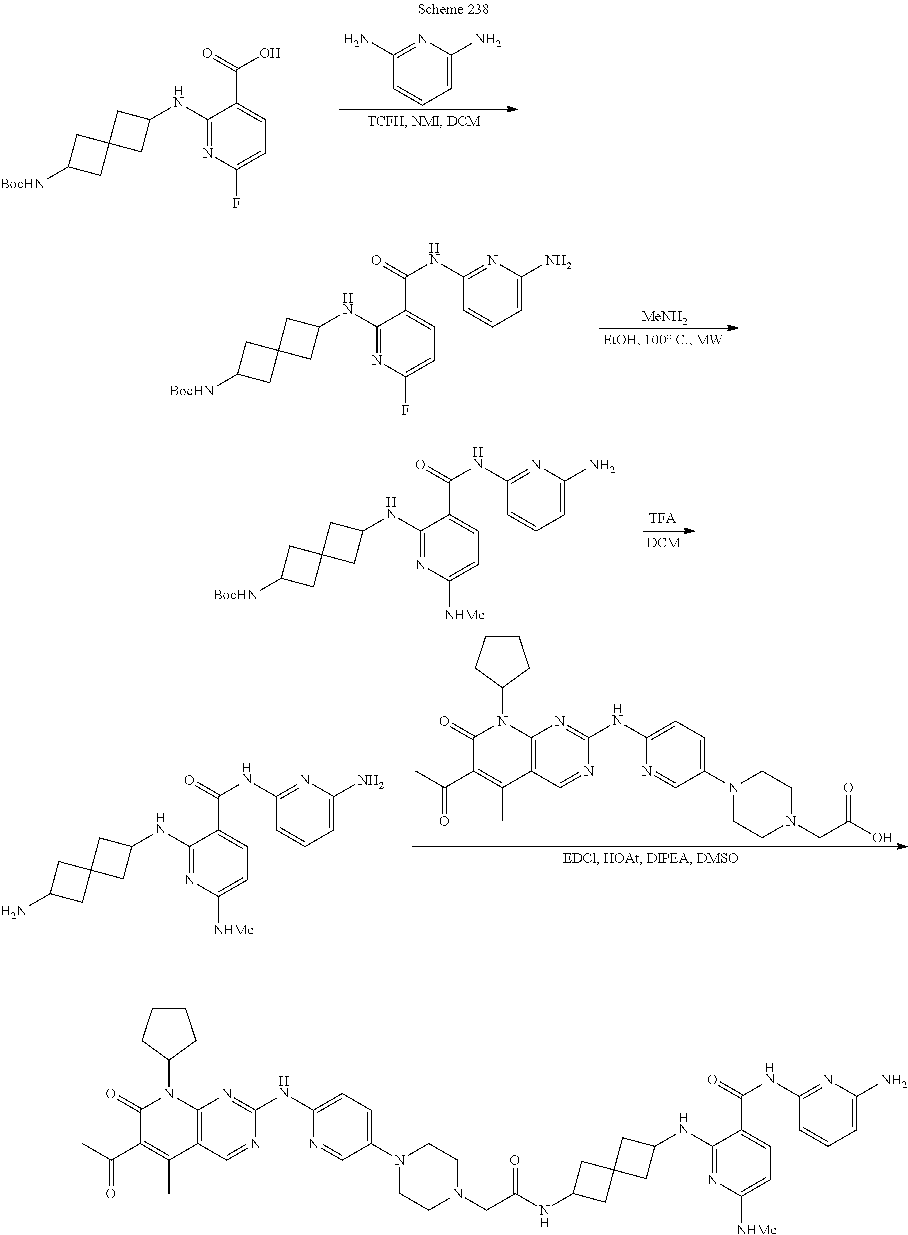

CPD-180 was synthesized following the standard procedures for preparing CPD-144 (1.8 mg, 5% yield over 4 steps) as a yellow solid. MS (ESI) m/z=855.9 [M+H]⁺.

### Example 239. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(6-(methylamino)pyridin-2-yl)nicotinamide (CPD-181)

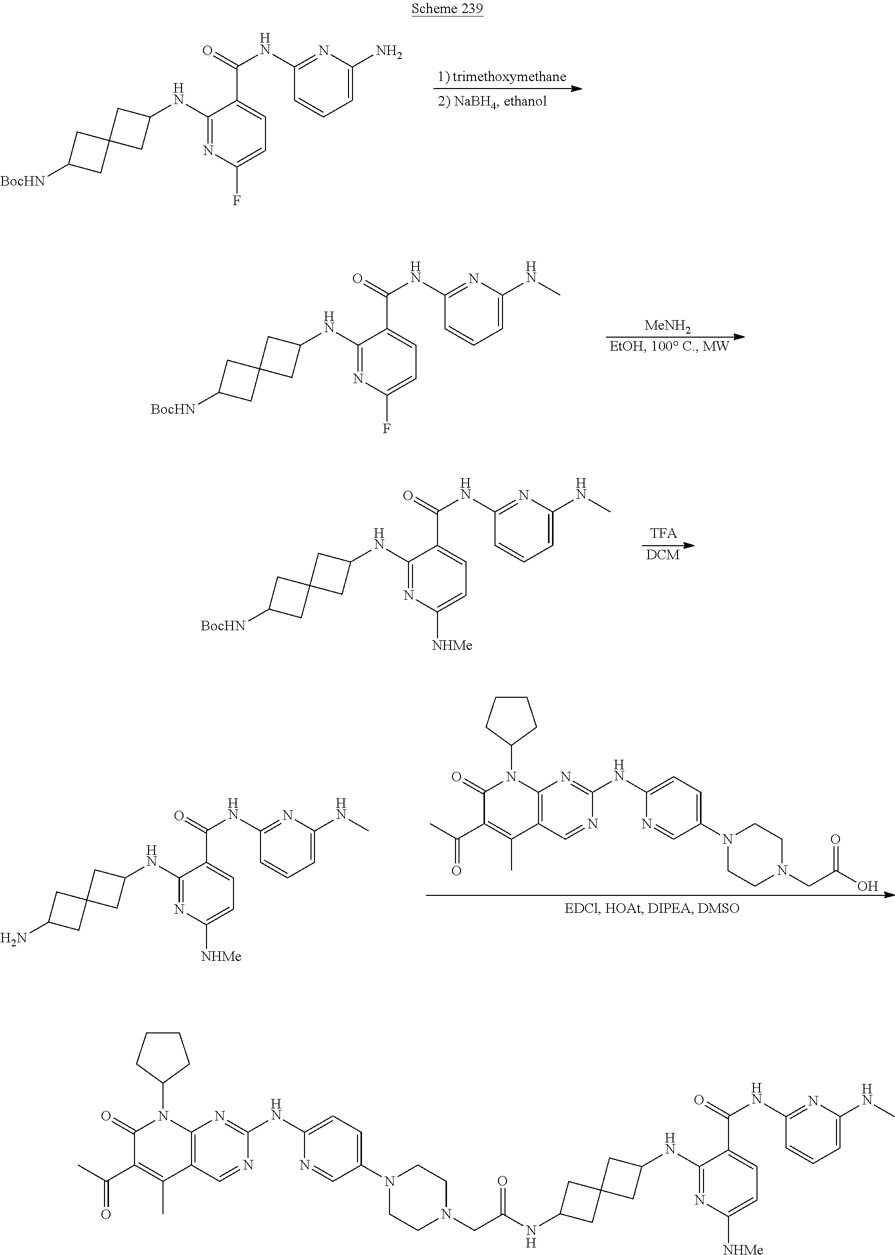

### Step 1. Synthesis of tert-butyl (6-((6-fluoro-3-((6-(methylamino)pyridin-2-yl)carbamoyl)pyridin-2-yl)amino)spiro[3.3]heptan-2-yl)carbamate

A solution of tert-butyl (6-((3-((6-aminopyridin-2-yl)carbamoyl)-6-fluoropyridin-2-yl)amino)spiro[3.3]heptan-2-yl)carbamate (30 mg, 65.72 μmol) in trimethoxymethane (2 mL) was stirred at 140 T for 0.5 h. After cooling down to rt, the solution was concentrated under reduced pressure and dried in vacuo for 30 min. The residue was dissolved in ethanol (5 mL), followed by adding NaBH₄ (24.3 mg, 0.66 mmol). The mixture was stirred at 90° C. for 1 h, before it was concentrated and purified by silica gel flash chromatography (PE/BA=3:1) to give the desired product (15 mg, 48% yield) as a yellow solid. MS (ESI) m/z=471.2 [M+H]⁺.

The remaining steps were performed following the standard procedures for preparing CPD-144 to provide the desired product (2.3 mg, 8% yield over 3 steps) as a light-yellow solid. MS (ESI) m/z=869.8 [M±H]⁺.

### Example 240. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5,6-dimethylpyridin-2-yl)-6-(methylamino)nicotinamide (CPD-182)

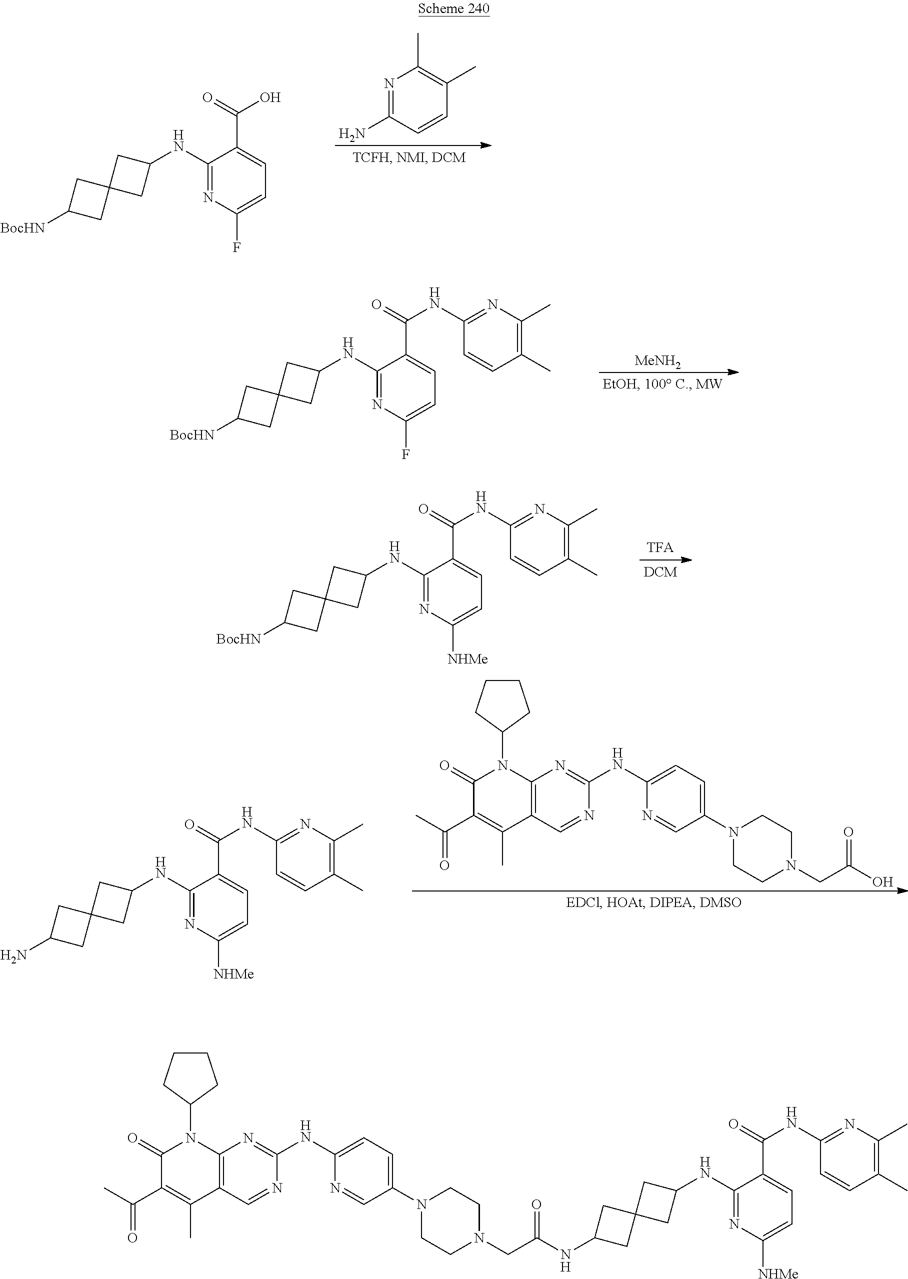

CPD-182 was synthesized following the standard procedures for preparing CPD-144 (3.5 mg, 4% yield over 3 steps) as a yellow solid. MS (ESI) m/z=868.8 [M+H]⁺.

### Example 241. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-1H-pyrrol-3-yl)-6-(methylamino)nicotinamide (CPD-183)

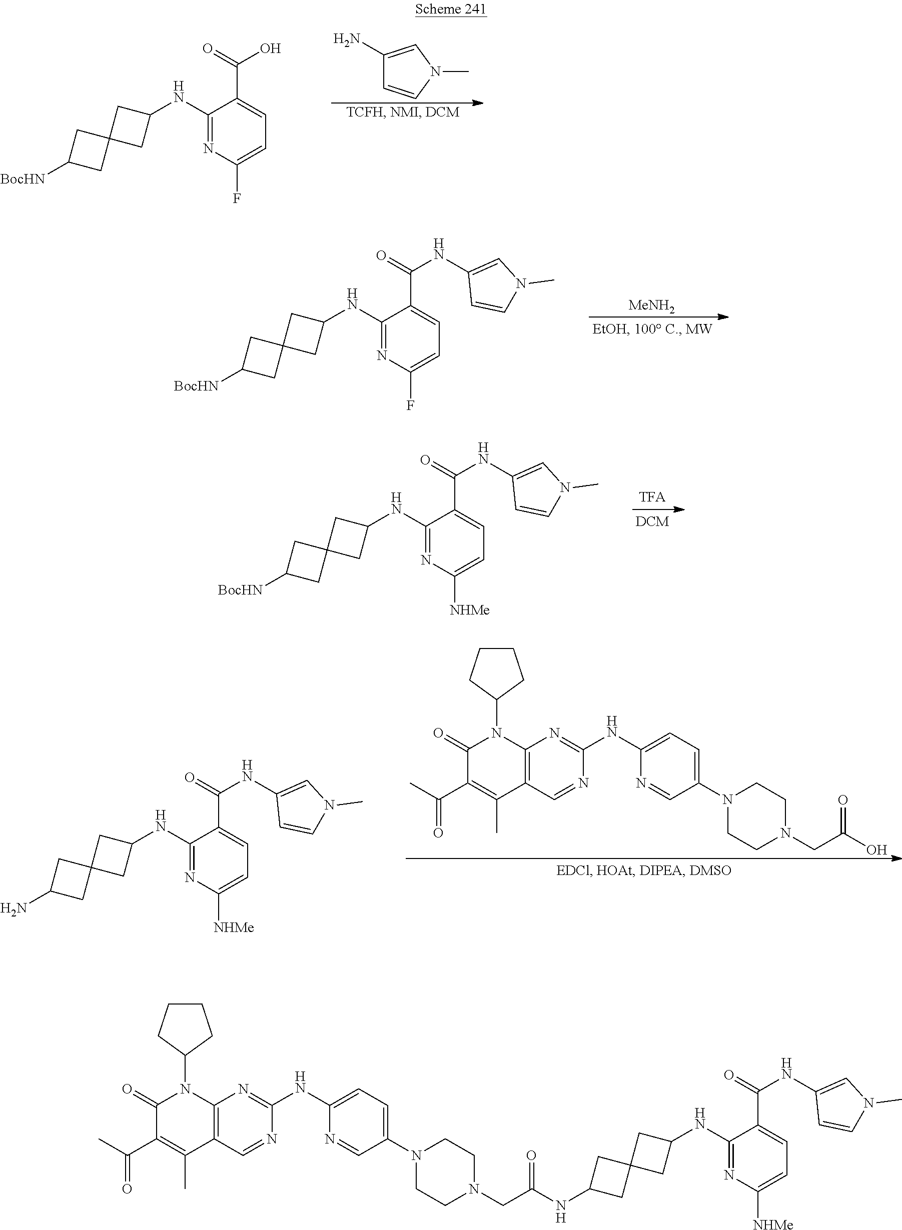

CPD-183 was synthesized following the standard procedures for preparing CPD-144 (19.6 mg, 32% yield over 4 steps) as a yellow solid. MS (ESI) m/z=842.9 [M+H]⁺.

### Example 242. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(difluoromethyl)pyridin-2-yl)-6-(methylamino)nicotinamide (CPD-184)

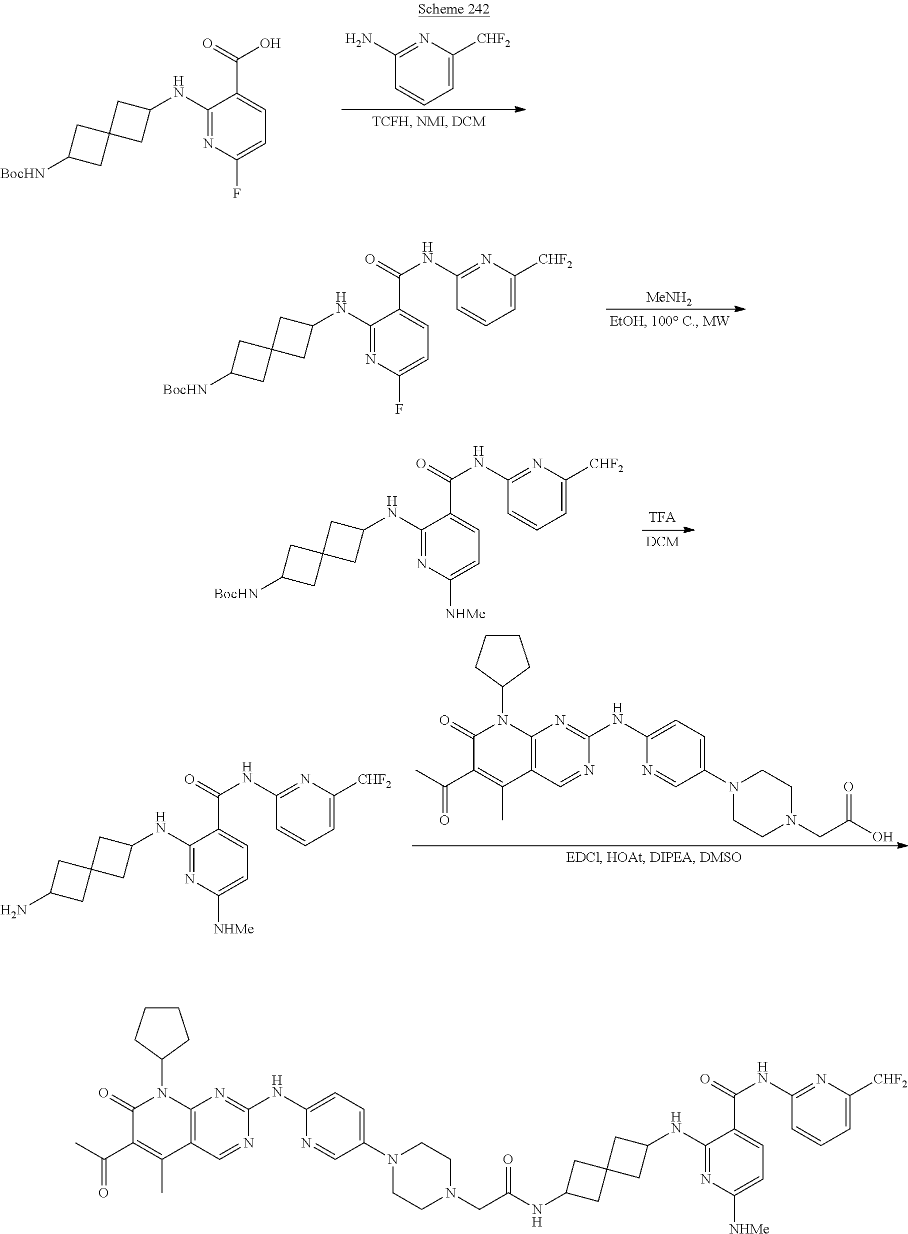

CPD-184 was synthesized following the standard procedures for preparing CPD-144 (8.0 mg, 2% yield over 4 steps) as a yellow solid. MS (ESI) m/z=890.8 [M+H]⁺.

### Example 243. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)cyclobutyl)amino)-N-(6-(dimethylamino)pyridazin-3-yl)-6-(methylamino)nicotinamide (CPD-185)

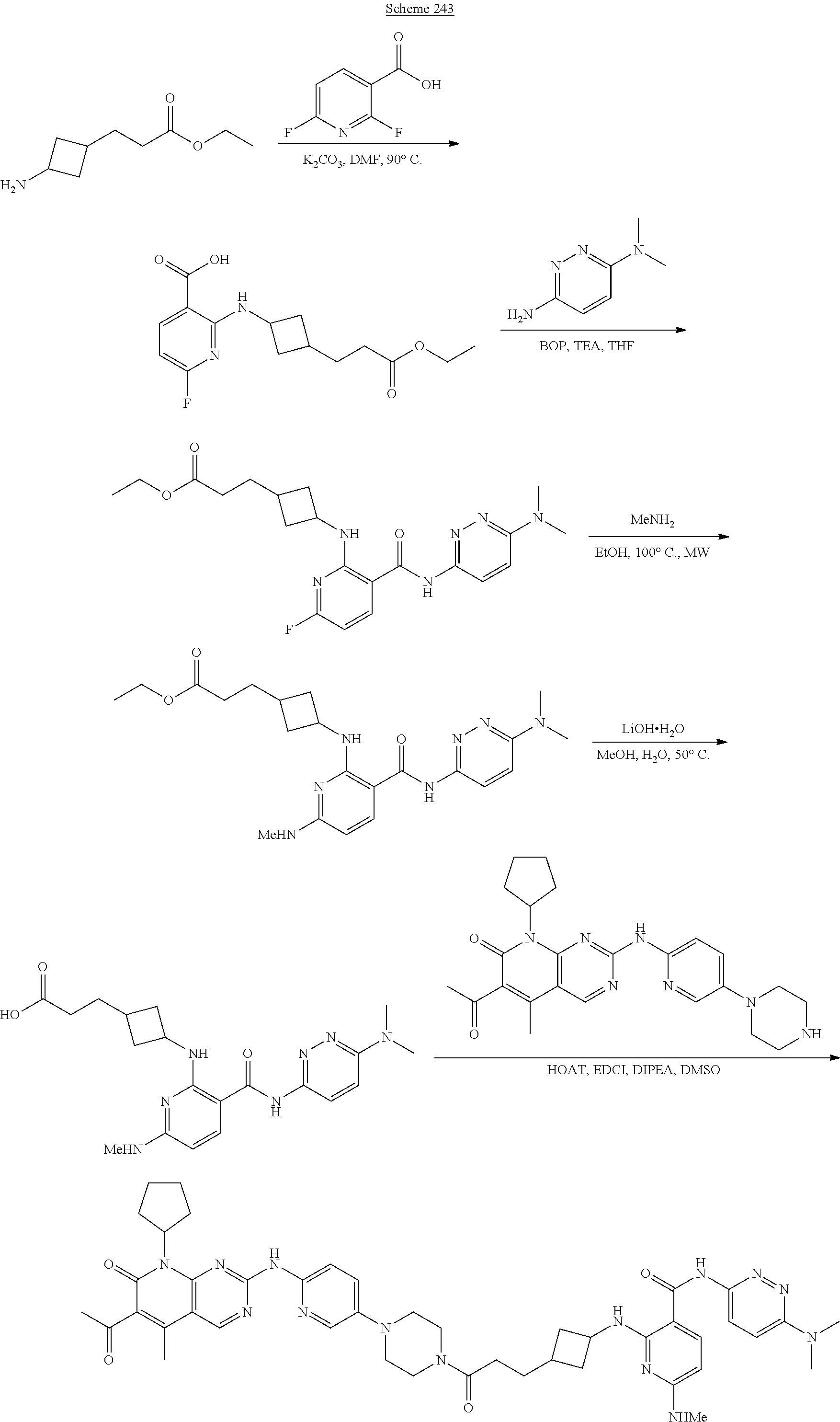

CPD-185 was synthesized following the standard procedures for preparing CPD-82 (4.0 mg, 10% yield over 5 steps) as a yellow solid. MS (ESI) m/z=843.7 [M+H]⁺.

### Example 244. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(6-(dimethylamino)pyridazin-3-yl)-6-(methylamino)nicotinamide (CPD-186)

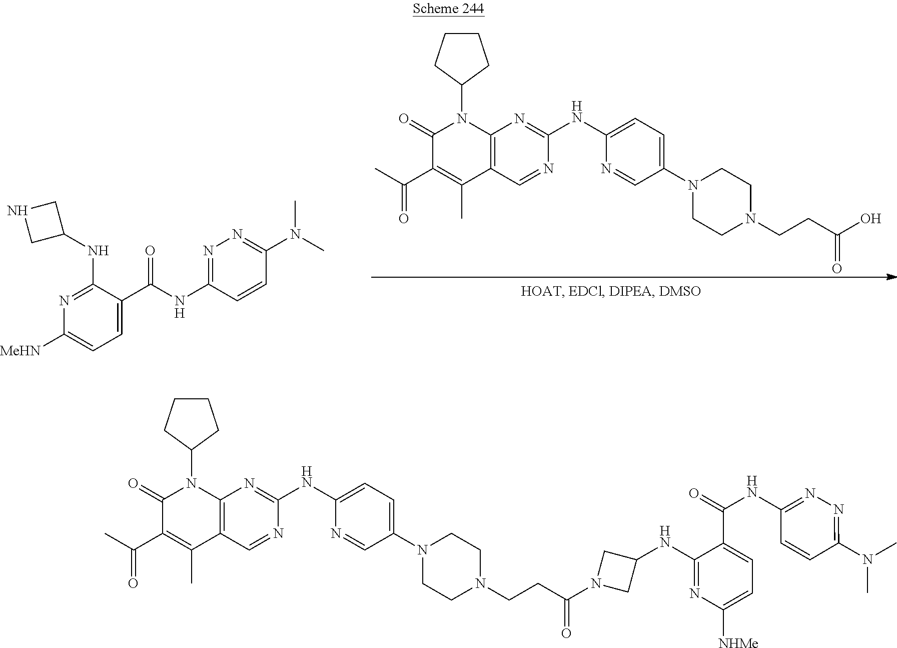

CPD-186 was synthesized following the standard procedure for preparing CPD-143 (6.0 mg, 16% yield) as a yellow solid. MS (ESI) m/z=844.8 [M+H]⁺.

### Example 245. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)azetidin-3-yl)amino)-N-(6-(dimethylamino)pyridazin-3-yl)-6-(methylamino)nicotinamide (CPD-187)

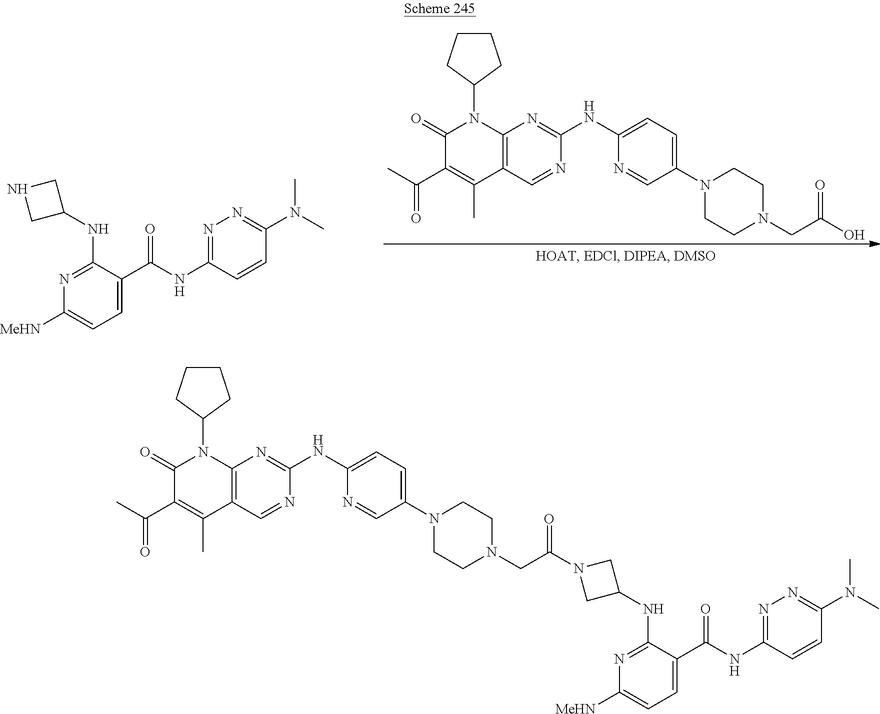 [SMILES: CNc1ccc(C(=O)Nc2ccc(N(C)C)nn2)c(NC2CNC2)n1 ; CC(=O)c1c(C)c2cnc(Nc3ccc(N4CCN(CC(=O)O)CC4)cn3)nc2n(C2CCCC2)c1=O ; CNc1ccc(C(=O)Nc2ccc(N(C)C)nn2)c(NC2CN(C(=O)CN3CCN(c4ccc(Nc5ncc6c(C)c(C(C)=O)c(=O)n(C7CCCC7)c6n5)nc4)CC3)C2)n1]

CPD-187 was synthesized following the standard procedure for preparing CPD-143 (6.0 mg, 17% yield) as a yellow solid. MS (ESI) m/z=830.8 [M+H]⁺.

### Example 246. 2-(((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)methyl)amino)-N-(6-(dimethylamino)pyridazin-3-yl)-6-(methylamino)nicotinamide (CPD-188)

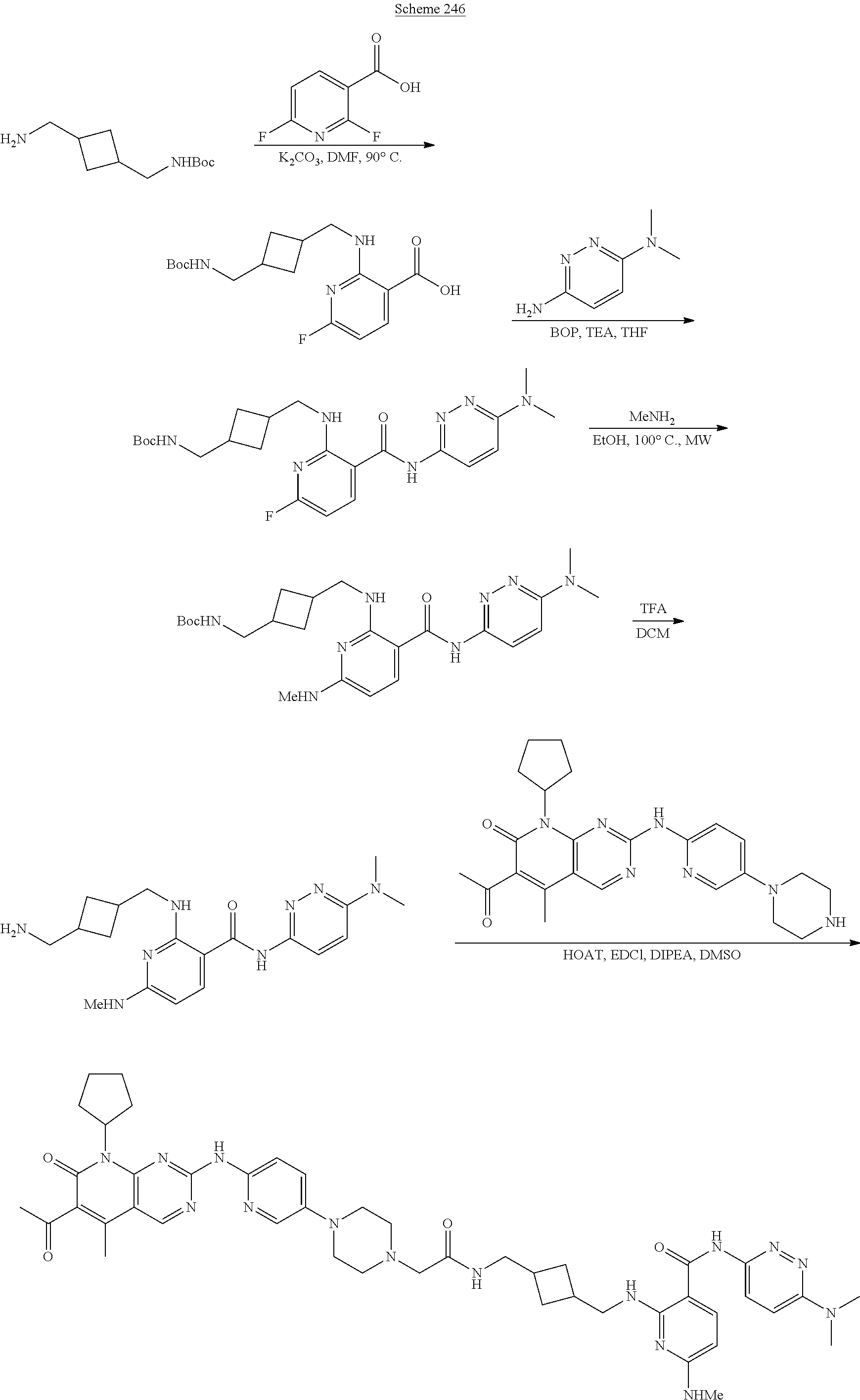

CPD-188 was synthesized following the standard procedures for preparing CPD-145 (5. 1 mg, 3% yield over 5 steps) as a yellow solid. MS (ESI) m/z=872.9 [M+H]⁺.

### Example 247. 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-1-oxopropan-2-yl)oxy)spiro[3.3]heptan-2-yl)amino)-N-(6-(dimethylamino)pyridazin-3-yl)-6-(methylamino)nicotinamide (CPD-189)

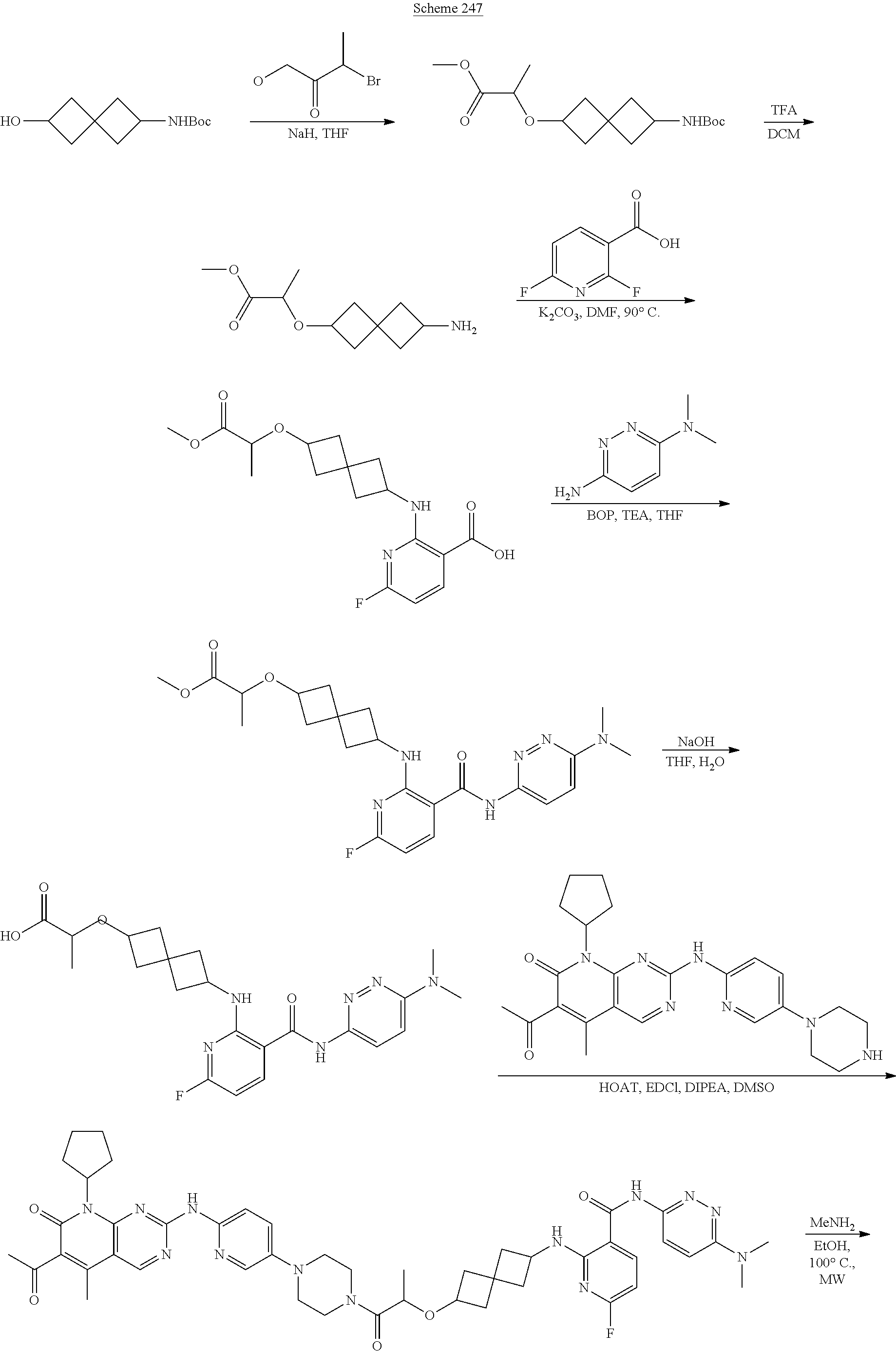

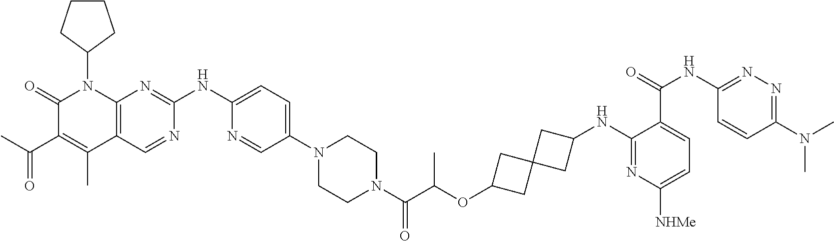 [SMILES: CNc1ccc(C(=O)Nc2ccc(N(C)C)nn2)c(NC2CC3(C2)CC(OC(C)C(=O)N2CCN(c4ccc(Nc5ncc6c(C)c(C(C)=O)c(=O)n(C7CCCC7)c6n5)nc4)CC2)C3)n1]

### Step 1. Synthesis of methyl 2-((6-((tert-butoxycarbonyl)amino)spiro[3. 3]heptan-2-yl)oxy)propanoate

To a solution of tert-butyl (6-hydroxyspiro[3.3]heptan-2-yl)carbamate (200 mg, 0.87 mmol) in THF (5 mL) was added NaH (30 mg, 1.32 mmol) in portions at 0 TC under N₂. After stirring at 0 [symbol] for 0.5 h, methyl 2-bromopropanoate (294 mg, 1.76 mmol) in THF (2 mL) was added dropwise at 0 [symbol] . After the resulting mixture was stirred at rt for 16 h, it was quenched with water (30 mL) slowly and extracted with ethyl acetate (3×20 mL). The organic phase was washed with brine, dried over anhydrous Na₂SO₄, filtered, and evaporated under reduced pressure. The residue was purified by silica gel flash chromatography to give the desired product (172 mg, 62%~yield) as a colorless oil.

### Step 2. Synthesis of methyl 2-((6-aminospiro[3. 3]heptan-2-yl)oxy)propanoate

A solution of methyl 2-((6-((tert-butoxycarbonyl)amino)spiro[3.3]heptan-2-yl)oxy)propanoate (172 mg, 0.55 mmol) in TEA (I mL) and dichloromethane (1 mL) was stirred at rt for 1 h. The solution was concentrated under vacuum to provide the crude product.

The remaining steps were performed following the standard procedures for preparing CPD-178 to provide the desired product (12.4 mg, 6% yield over 5 steps) as a yellow solid. MS (EST) m/z 899.9 [M+H]⁺.

### Example 248. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(6-methylpyridin-2-yl)nicotinamide (CPD-190)

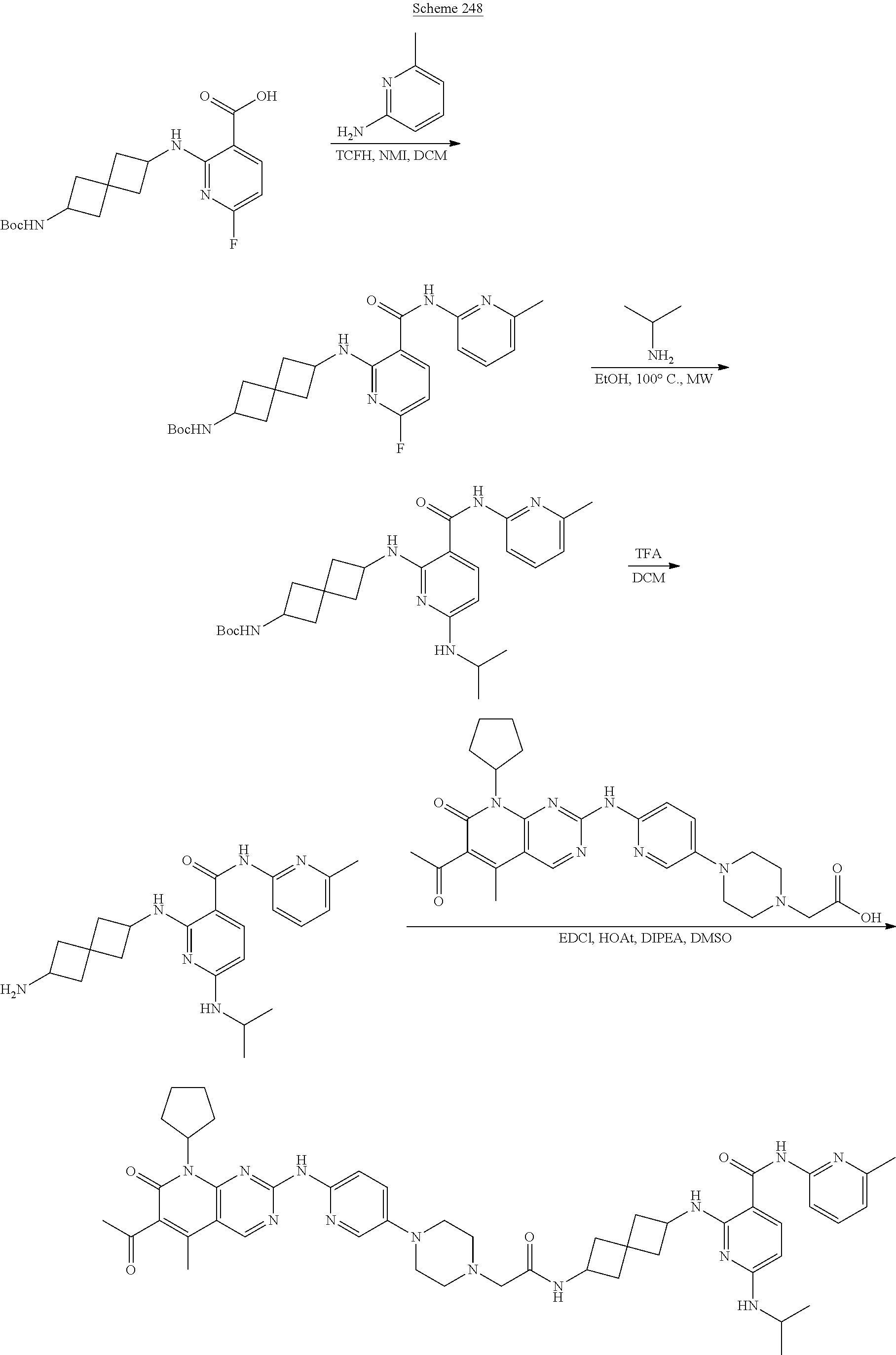

CPD-190 was synthesized following the standard procedures for preparing CPD-144 (3.2 mg, 4% yield over 4 steps) as a yellow solid. MS (ESI) m/z=882.9 [M+H]⁺.

### Example 249. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-methyl-N-(6-methylpyridin-2-yl)nicotinamide (CPD-191)

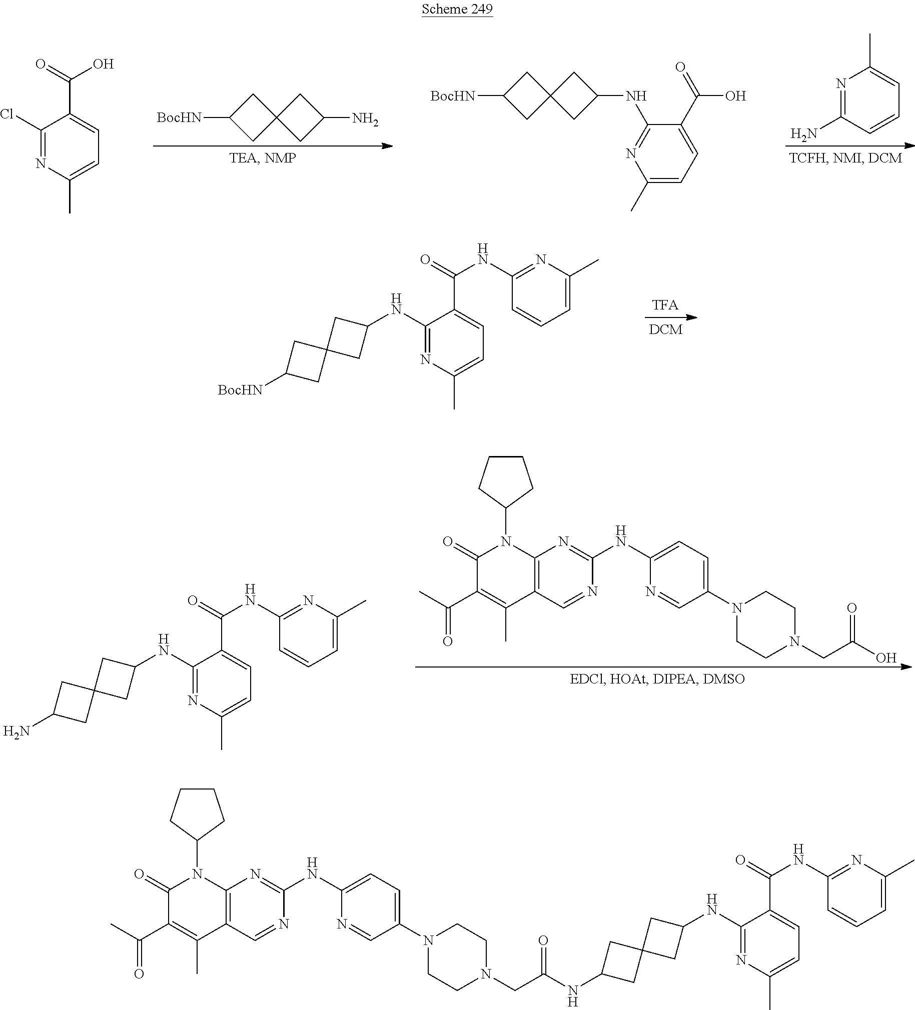

### Step 1. Synthesis of 2-((6-((tert-butoxycarbonyl)amino)spiro[3.3]heptan-2-yl)amino)-6-methylnicotinic acid

To a solution of 2-chloro-6-methylnicotinic acid (91.0 mg, 0.53 mmol), tert-butyl (6-aminospiro[3.3]heptan-2-yl)carbamate (120 mg, 0.53 mmol) in NMP (2 mL) was added TEA (0.3 mL). The reaction mixture was heated at 170 [symbol] under microwave irradiation for 3 h. After cooling down to rt, the mixture was purified by reverse-phase chromatography to give the desired product (90 mg, 470% yield) as a yellow solid. MS (ESI) m/z=362.7 [M+H]⁺.

The remaining steps were performed following the standard procedures for preparing CPD-144 to provide the desired product (1.8 mg, 6% yield over 3 steps) as a light-yellow solid. MS (ESI) m/z=839.8 [M+H]⁺.

### Example 250. ((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-methyl-N-(5-methylisoxazol-3-yl)nicotinamide (CPD-192)

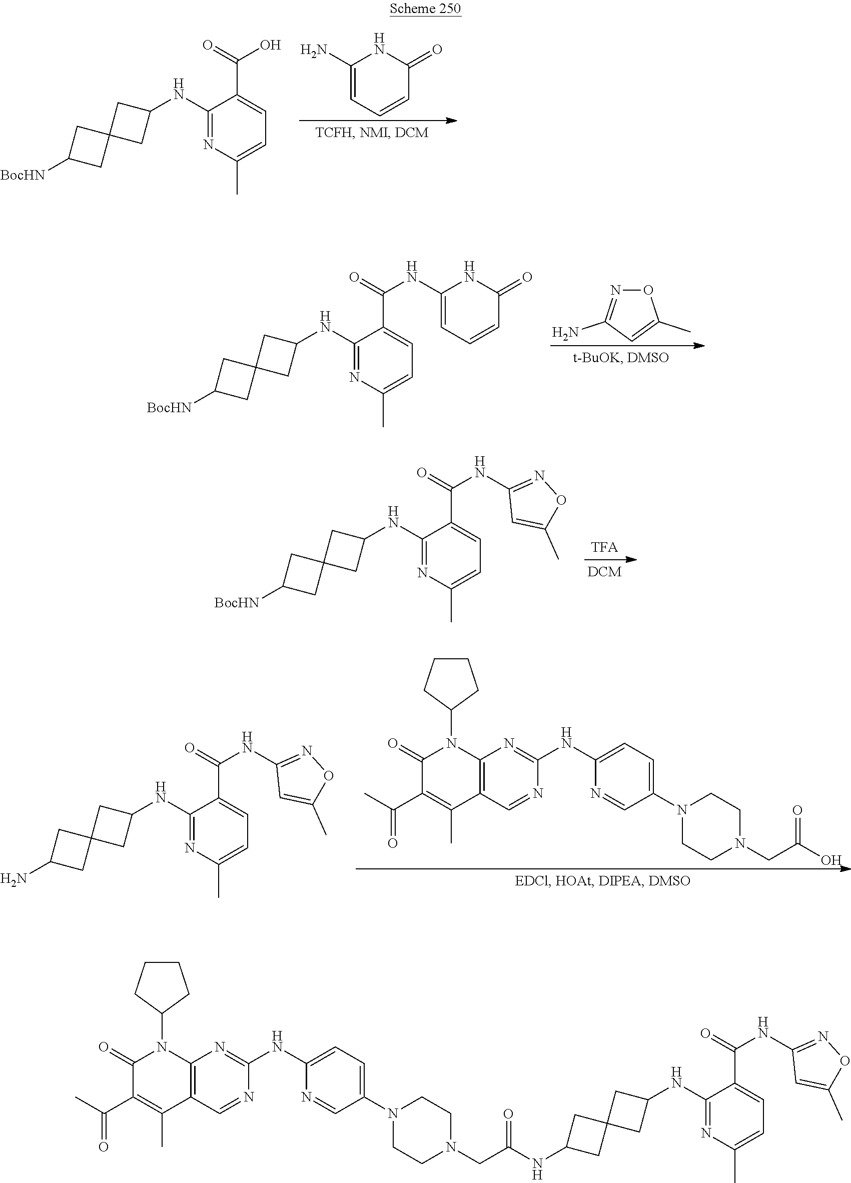

CPD-192 was synthesized following the standard procedures for preparing CPD-159 (2.1 mg, 3% yield over 4 steps) as a yellow solid. MS (ESI) m/z=829.8 [M+H]⁺.

### Example 251. 2-(((cis-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(6-(dimethylamino)pyridazin-3-yl)-6-(methylamino)nicotinamide (CPD-193)

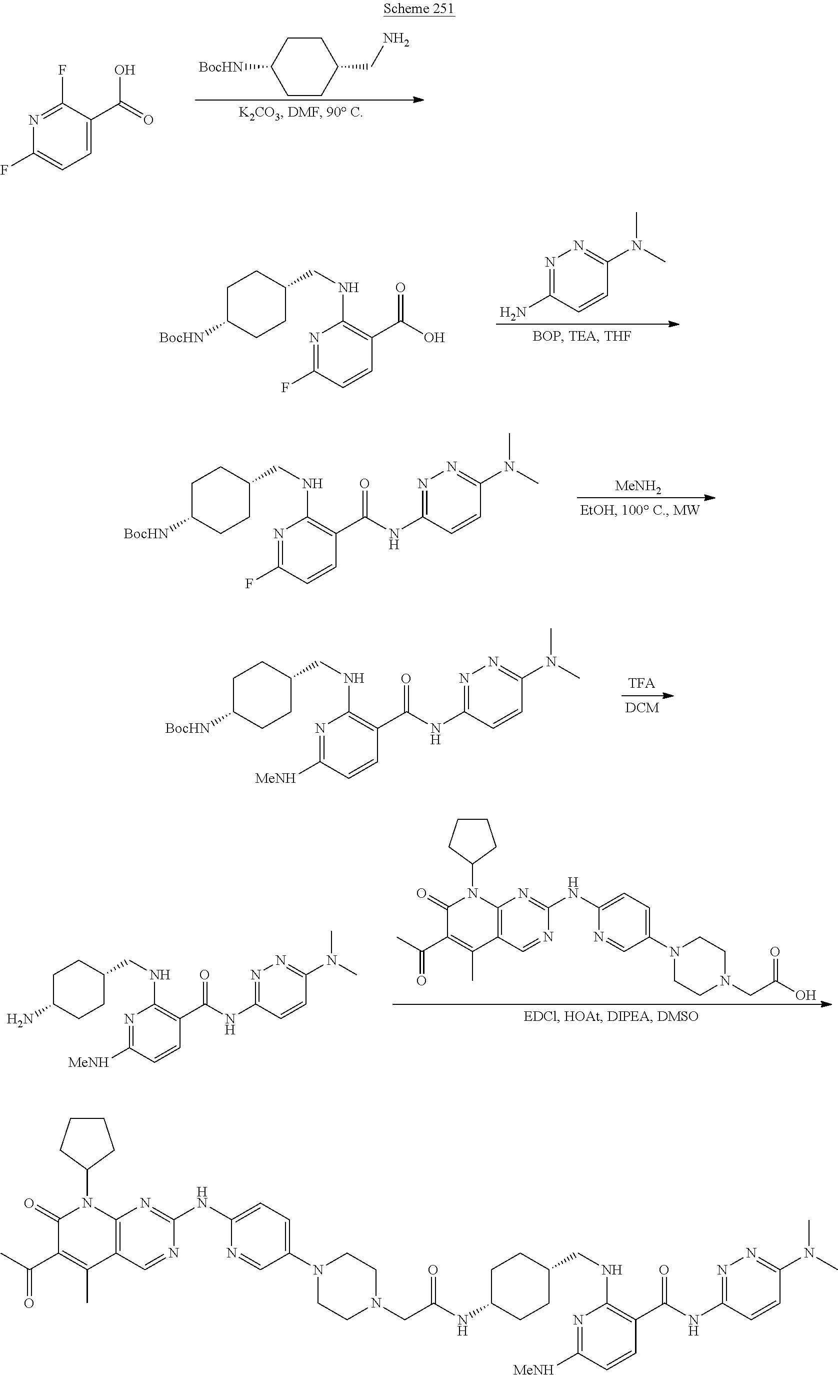

CPD-193 was synthesized following the standard procedures for preparing CPD-145 (2.0 mg, 2% yield over 5 steps) as a yellow solid. MS (ESI) m/z=886.8 [M+H]⁺.

### Example 252. 2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)-N-(6-(dimethylamino)pyridazin-3-yl)-6-(methylamino)nicotinamide (CPD-194)

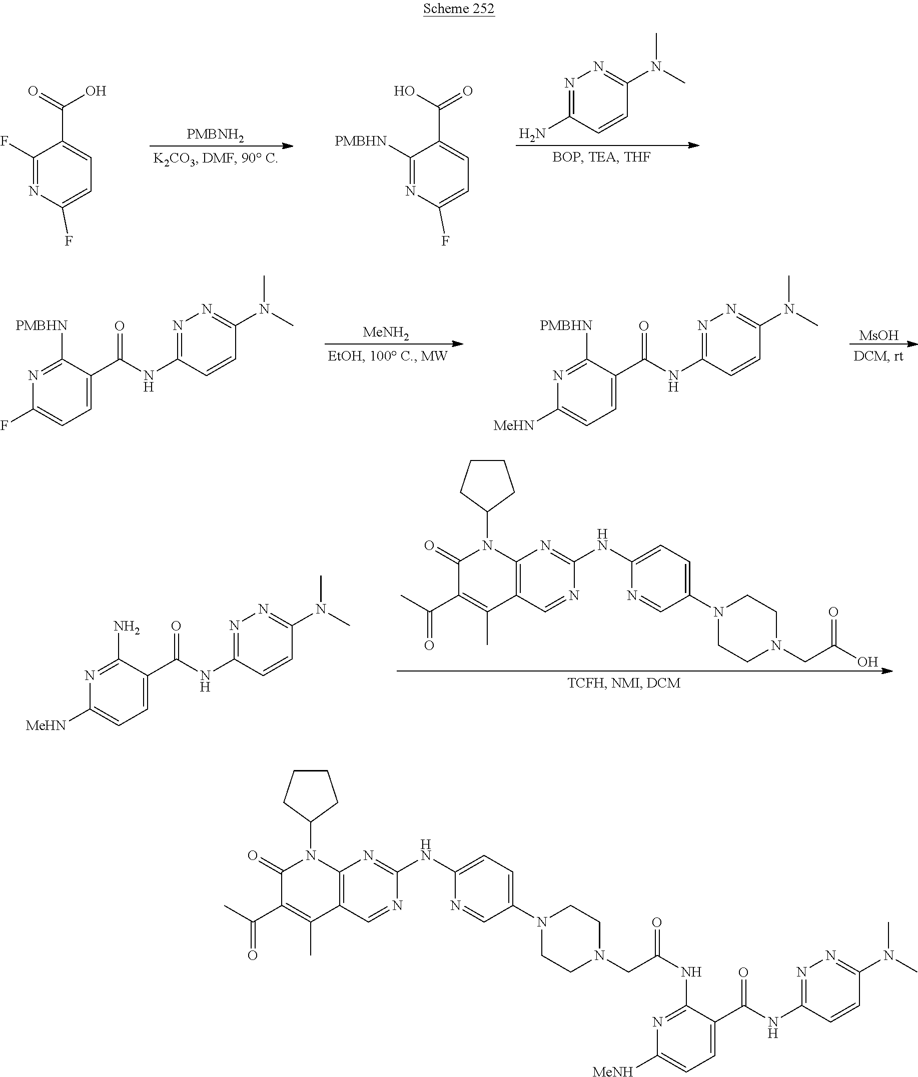

CPD-194 was synthesized following the standard procedures for preparing CPD-145 (2.0 mg, 1% yield over 4 steps) as a yellow solid. MS (ESI) m/z=775.7 [M+H]⁺.

### Example 253. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-isopropyl-1-methyl-1H-imidazol-4-yl)-6-(methylamino)nicotinamide (CPD-195)

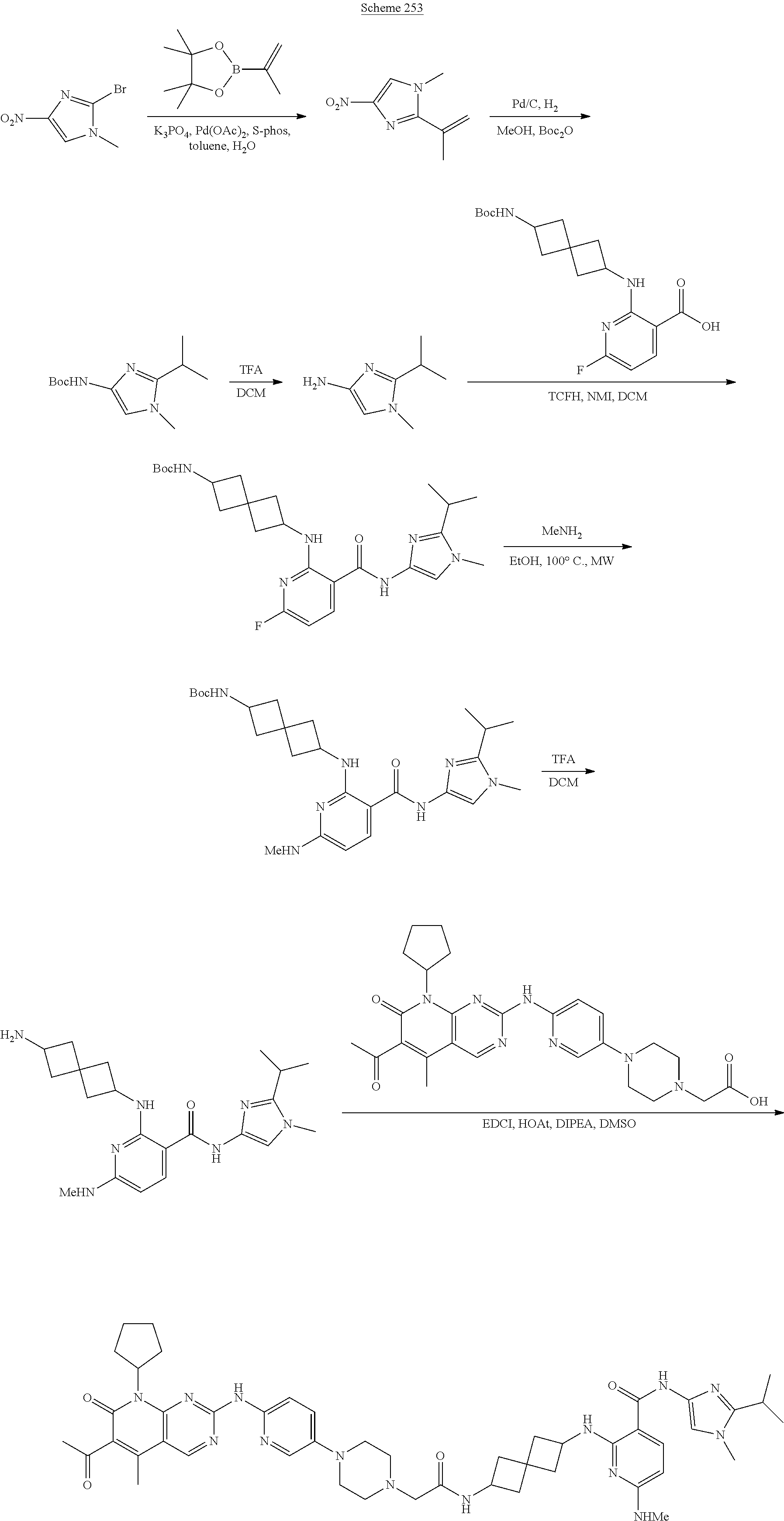

### Step 1. Synthesis of 1-methyl-4-nitro-2-(prop-1-en-2-yl)-1H-imidazole

To a mixture of 2-bromo-1-methyl-4-nitro-1H-imidazole (30 mg, 0.14 mmol) and 4,4,5,5-tetramethyl-2-(prop-1-en-2-yl)-1,3,2-dioxaborolane (122 mg, 0.7 mmol) in toluene (5 mL) and H₂O (1 mL) were added K₃PO₄ (92 mg, 0.4 mmol), S-phos (6 mg, 0.01 mmol) and Pd(OAc)₂ (3 mg, 0.01 mmol) at rt under N₂. After stirring at 100° C. for 16 h, the reaction mixture was filtered and concentrated. The resulting residue was purified by silica gel flash chromatography to give the desired product (21 mg, 86% yield) as a colorless oil. MS (ESI) m/z=168.3 [M+H]⁺.

### Step 2. Synthesis of tert-butyl (2-isopropyl-1-methyl-1H-imidazol-4-yl)carbamate

To a solution of 1-methyl-4-nitro-2-(prop-1-en-2-yl)-1H-imidazole (21 mg, 0.12 mmol) in MeOH (2 mL) were added BOC₂O (274 mg, 1.26 mmol) and 10% Pd/C (14 mg). The reaction mixture was stirred at rt for 3 h under hydrogen balloon. The reaction mixture was filtered and concentrated. The filtrate was purified by silica gel flash chromatography to give the desired product (23 mg, 76% yield) as a colorless oil. MS (ESI) m/z=240.6 [M+H]⁺.

### Step 3. Synthesis of 2-isopropyl-1-methyl-1H-imidazol-4-amine

A solution of tert-butyl (2-isopropyl-1-methyl-1H-imidazol-4-yl)carbamate (23 mg, 0.09 mmol) in TFA (1 mL) and dichloromethane (2 mL) was stirred at rt for 1 h. The solution was concentrated and dried under vacuum to give the crude product.

The remaining steps were performed following the standard procedures for preparing CPD-144 to provide the desired product (8.1 mg, 12% yield over 4 steps) as a yellow solid. MS (ESI) m/z=885.8 [M+H]⁺.

### Example 254. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-6-(methylamino)-N-(6-methylpyridin-2-yl)nicotinamide (CPD-196)

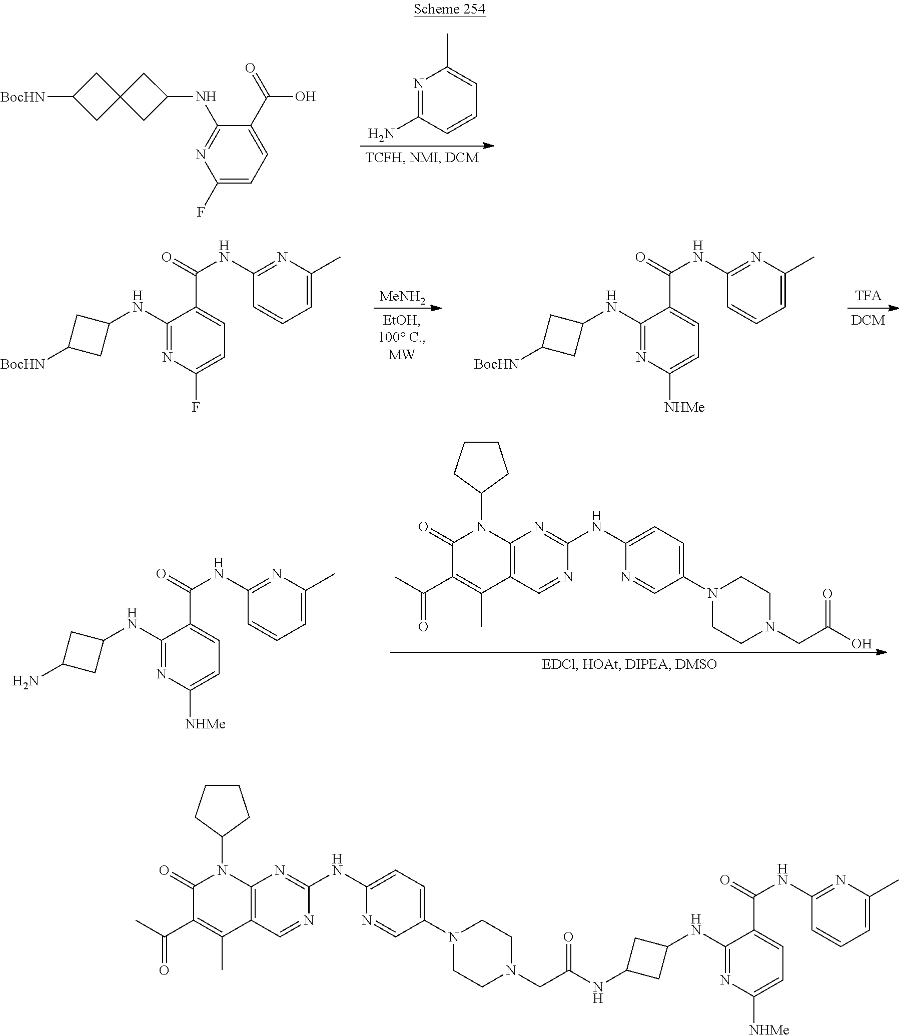

CPD-196 was synthesized following the standard procedures for preparing CPD-144 (1.9 mg, 3% yield over 4 steps) as a yellow solid. MS (ESI) m/z=814.8 [M+H]⁺.

### Example 255. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(1,5-dimethyl-1H-pyrazol-3-yl)-6-(methylamino)nicotinamide (CPD-197)

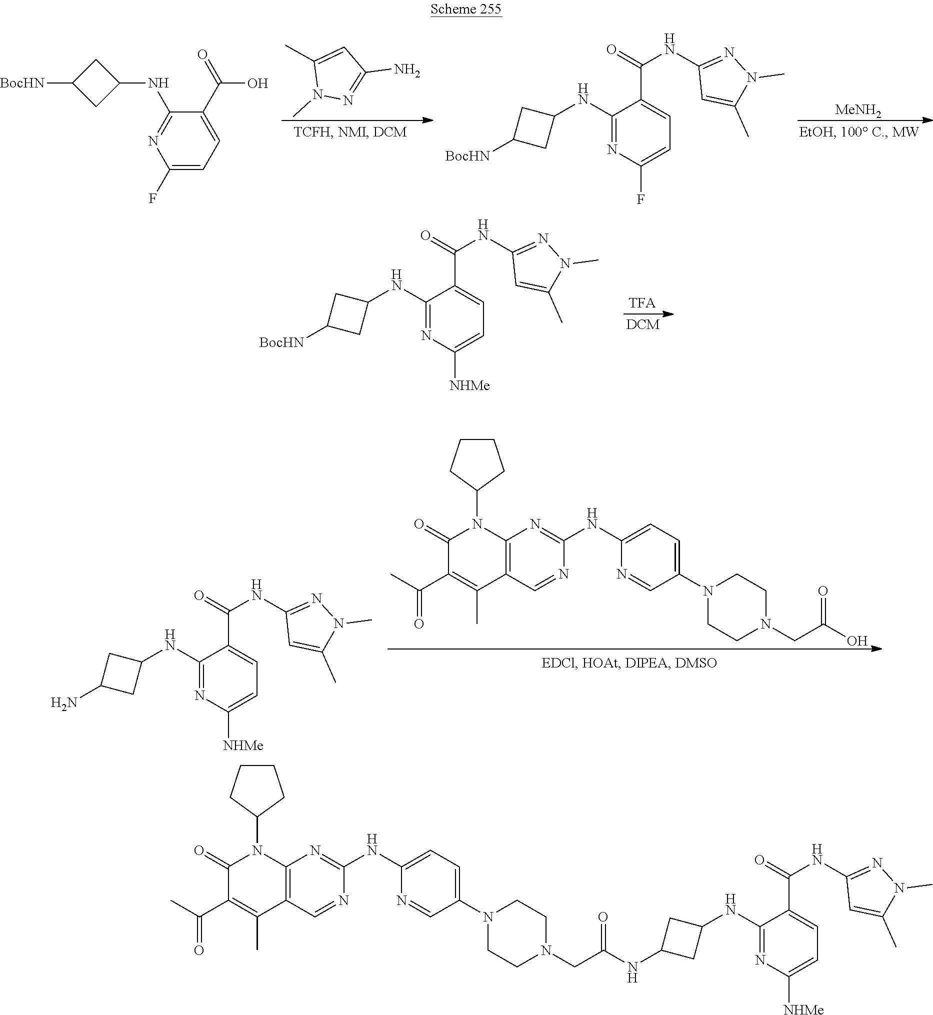

CPD-197 was synthesized following the standard procedures for preparing CPD-144 (1.6 mg, 3% yield over 4 steps) as a yellow solid. MS (ESI) m/z=817.8 [M+H]⁺.

### Example 256. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-6-(methylamino)-N-(5-methylisoxazol-3-yl)nicotinamide (CPD-198)

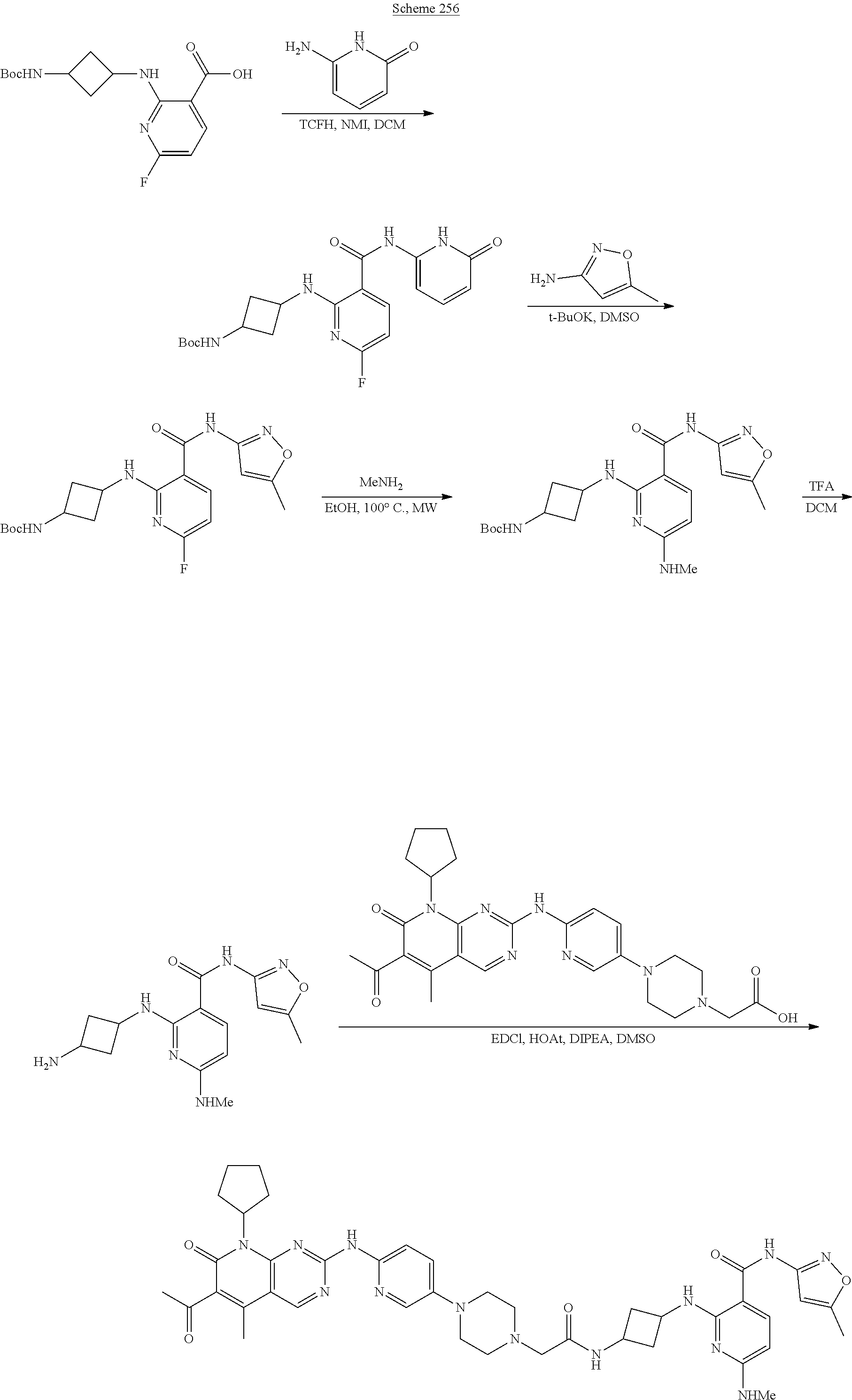

CPD-198 was synthesized following the standard procedures for preparing CPD-159 (1.7 mg, 2% yield over 5 steps) as a yellow solid. MS (ESI) m/z=804.8 [M±H]⁺.

### Example 257. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(5-methylisoxazol-3-yl)nicotinamide (CPD-199)

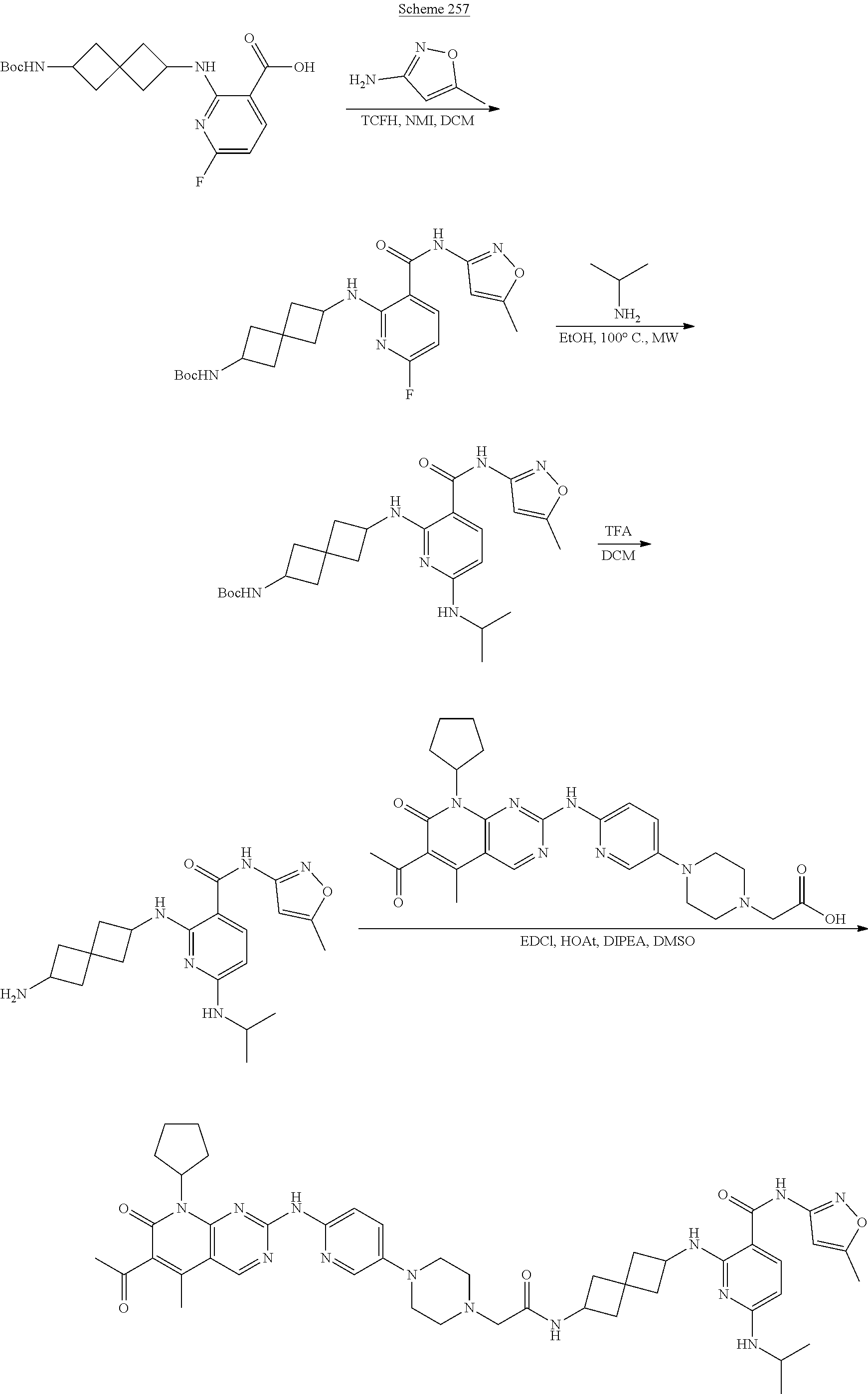

CPD-199 was synthesized following the standard procedures for preparing CPD-144 (3.0 mg, 5% yield over 4 steps) as a yellow solid. MS (ESI) m/z=872.8 [M+H]⁺.

### Example 258. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-(methylamino)nicotinamide (CPD-200)

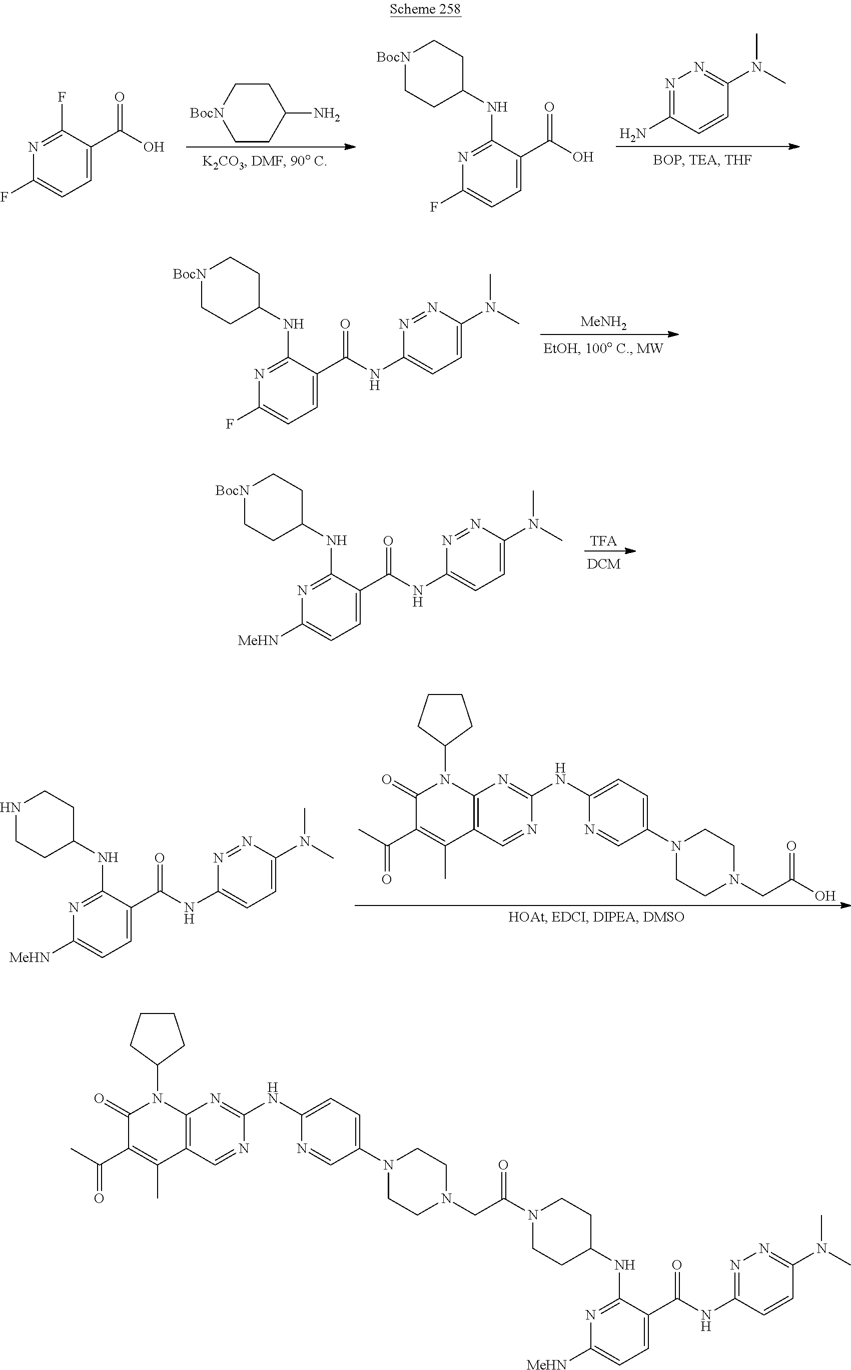

CPD-200 was synthesized following the standard procedures for preparing CPD-145 (6.8 mg, 5% yield over 5 steps) as a yellow solid. MS (ESI) m/z=858.8 [M+H]⁺.

### Example 259. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)pyrrolidin-3-yl)amino)-N-(6-(dimethylamino)pyridazin-3-yl)-6-(methylamino)nicotinamide (CPD-201)

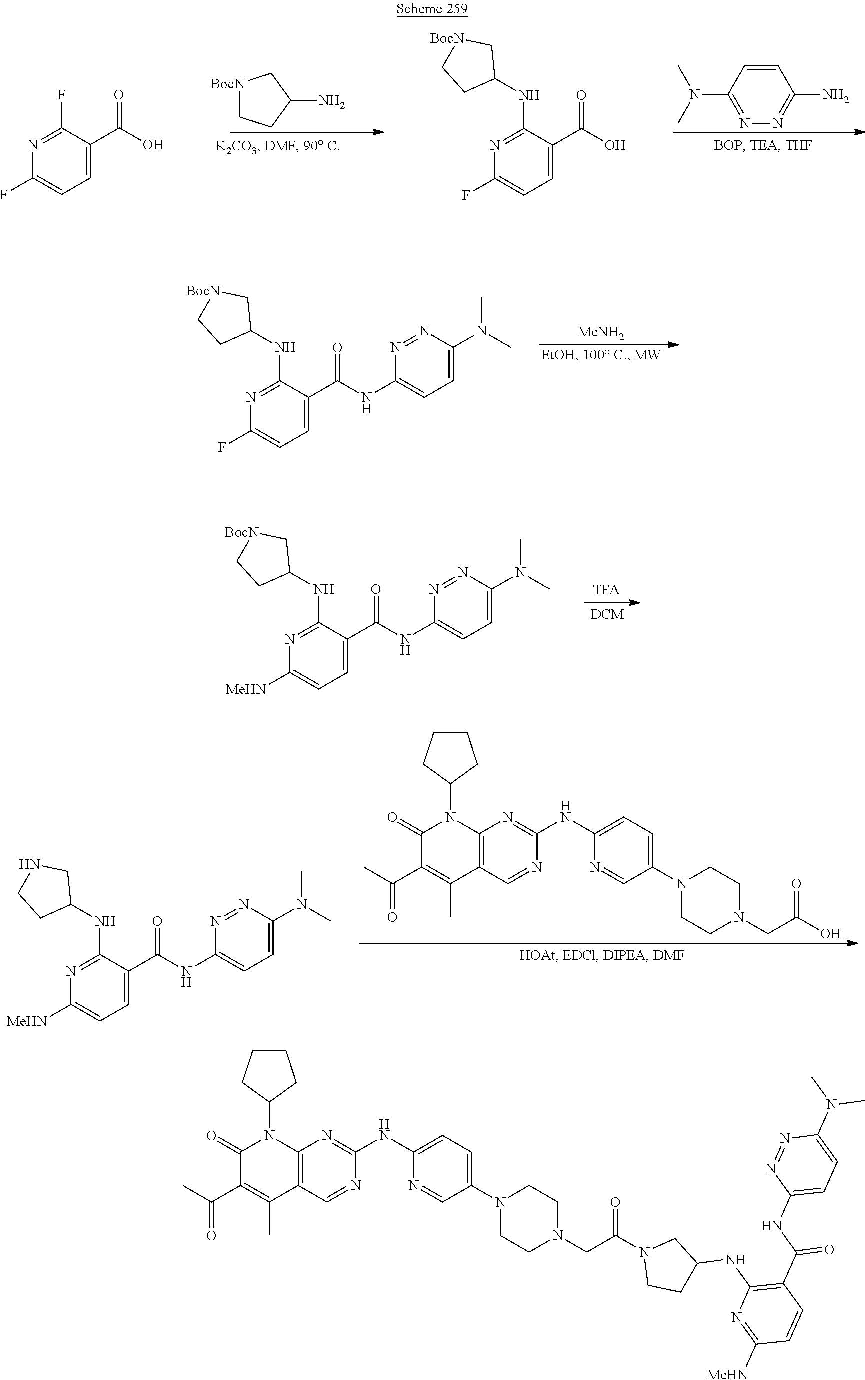

CPD-201 was synthesized following the standard procedures for preparing CPD-145 (6.8 mg, 9% yield over 5 steps) as a yellow solid. MS (ESI) m/z=844.8 [M+H]⁺.

### Example 260. 2-((3-((4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)methyl)cyclobutyl)amino)-N-(6-(dimethylamino)pyridazin-3-yl)-6-(methylamino)nicotinamide (CPD-202)

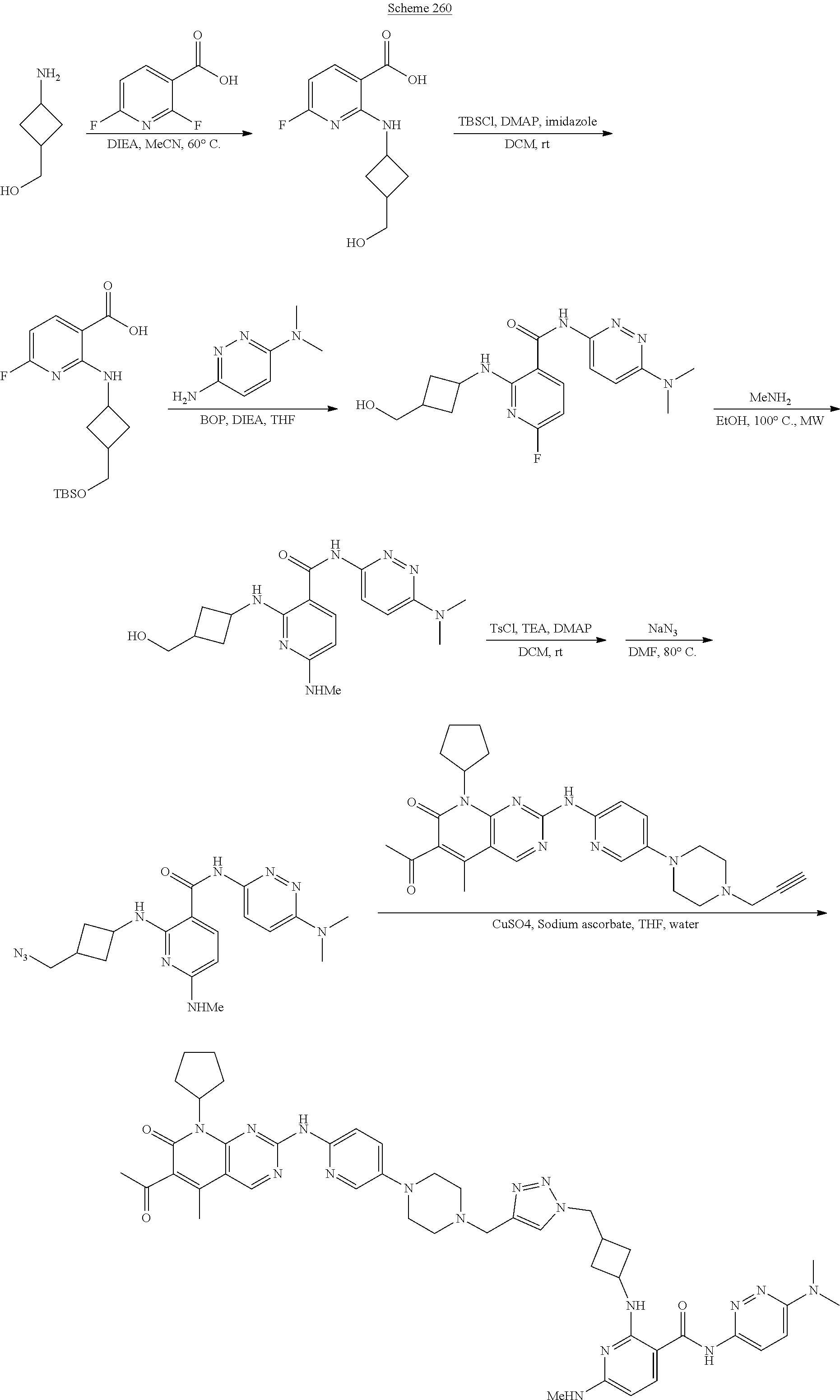

### Step 1. Synthesis of 6-fluoro-2-[[3-(hydroxymethyl)cyclobutyl]amino]pyridine-3-carboxylic acid

To a solution of (3-aminocyclobutyl)methanol (2.00 g, 19.8 mmol) and 2,6-difluoropyridine-3-carboxylic acid (3.18 g, 20.0 mmol) in acetonitrile (20 mL) was added DIEA (12.8 g, 98.9 mmol, 17.2 mL). The mixture was stirred at 60 [symbol] for 12 h. After that, the mixture was concentrated under reduced pressure. The residue was dissolved in aq. NaOH solution (1 N, 30 mL) and the solution was stirred at 40 [symbol] for 12 h. After cooling to rt, the reaction mixture was extracted with ethyl acetate (30 mL×4). The aqueous phase was adjusted pH to 3-4 by aq. HCl (6 N). The mixture was extracted with CH₂Cl₂/CH₃OH (5:1, 30 mL×6). The combined organic layers were dried over Na₂SO₄, filtered, and concentrated under reduced pressure to provide the desired product (3.50 g, 74% yield) as a colorless oil. MS (ESI) m/z=241.3 [M+H]⁺.

### Step 2. Synthesis of 2-[[3-[[tert-butyl(dimethyl)silyl]oxymethyl]cyclobutyl]amino]-6-fluoro-pyridine-3-carboxylic acid

To a solution of 6-fluoro-2-[[3-(hydroxymethyl)cyclobutyl]amino]pyridine-3-carboxylic acid (3.50 g, 14.6 mmol) in dichloromethane (35 mL) were added DMAP (2.14 g, 17.5 mmol), imidazole (5.95 g, 87.4 mmol) and TBSCl (3.29 g, 21.9 mmol, 2.7 mL). The mixture was stirred at rt for 2 h. The reaction mixture was diluted with dichloromethane (20 mL) and washed with sat. K₂CO₃ (20 mL×3). The organic layer was dried over Na₂SO₄, filtered, and concentrated under reduced pressure to provide the crude product (5.00 g, crude) as a yellow oil. MS (ESI) m/z=355.4 [M+H]⁺.

### Step 3. Synthesis of N-[6-(dimethylamino)pyridazin-3-yl]-6-fluoro-2-[[3-(hydroxymethyl)cyclobutyl]amino]pyridine-3-carboxamide

To a solution of 2-[[3-[[tert-butyl(dimethyl)silyl]oxymethyl]cyclobutyl]amino]-6-fluoro-pyridine-3-carboxylic acid (5.00 g, 14.1 mmol) in THF (50 mL) were added BOP (8.11 g, 18.3 mmol) and DIEA (5.47 g, 42.3 mmol, 7.4 mL). The mixture was stirred at rt for 12 h, before it was concentrated under reduced pressure. The residue was diluted with water (200 mL) and extracted with ethyl acetate (100 mL×5). The combined organic layers were washed with brine, dried over Na₂SO₄, filtered, and concentrated under reduced pressure. The residue was purified by reversed-phase HPLC (0.10% TFA condition) to provide the desired product (1.00 g, 20% yield) as a yellow solid. MS (ESI) m/z=361.4 [M+H]⁺.

### Step 4. Synthesis of [3-[[3-[[6-(dimethylamino)pyridazin-3-yl]carbamoyl]-6-(methylamino)-2-pyridyl]amino]cyclobutyl]methyl 4-methylbenzenesulfonate

To a solution of N-[6-(dimethylamino)pyridazin-3-yl]-6-fluoro-2-[[3-(hydroxymethyl)cyclobutyl]amino]pyridine-3-carboxamide (0.50 g, 1.39 mmol) in EtOH (10 mL) were added TEA (1.40 g, 13.9 mmol, 1.93 mL) and methylamine hydrochloride (937 mg, 13.9 mmol). The mixture was stirred at 120 CC for 2 h. The reaction mixture was concentrated under reduced pressure. The residue was diluted with H₂O (30 mL) and extracted with ethyl acetate (30 mL×3). The combined organic layers were washed with brine, dried over Na₂SO₄, filtered, and concentrated under reduced pressure to provide the desired product (800 mg, 78% yield) as a yellow solid. MS (ESI) m/z=372.7 [M+H]⁺.

### Step 5. Synthesis of N-[6-(dimethylamino)pyridazin-3-yl]-2-[[3-(hydroxymethyl)cyclobutyl]amino]-6-(methylamino)pyridine-3-carboxamide

To a solution of N-[6-(dimethylamino)pyridazin-3-yl]-2-[[3-(hydroxymethyl)cyclobutyl]amino]-6-(methylamino)pyridine-3-carboxamide (400 mg, 1.08 mmol) in dichloromethane (5 mL) were added TEA (218 mg, 2.15 mmol, 300 μL), DMAP (13 mg, 108 gmol) and 4-methylbenzenesulfonyl chloride (226 mg, 1.18 mmol) at 0 [symbol] . The mixture was stirred at rt for 2 h. The reaction mixture was quenched with sat. NaHCO₃ (20 mL) and extracted with dichloromethane (20 mL×3). The combined organic layers were dried over Na₂SO₄, filtered, and concentrated under reduced pressure to give a residue. The residue was purified by silica gel column chromatography (ethyl acetate/petroleum ether=9:1 to 1:1) to provide the desired product (230 mg, 19% yield) as a yellow solid. ¹HNMR (400 MHz, CDCl₃) δ 8.56 (d, J=4.8 Hz, 1H), 8.02 (d, J=9.6 Hz, 1H), 7.73 (d, J=8.0 Hz, 2H), 7.65 (d, J=8.4 Hz, 1H), 7.27 (d, J=8.4 Hz, 2H), 6.82 (d, J=10.0 Hz, 1H), 5.59 (d, J=8.8 Hz, 1H), 4.54-4.39 (m, 1H), 3.05 (s, 6H), 2.83 (d, J=3.6 Hz, 3H), 2.47 (s, 1H), 2.37 (s, 3H), 2.19-2.09 (m, 2H), 2.07-2.00 (m, 2H). MS (ESI) m/z=526.4 [M+H]⁺.

### Step 6. Synthesis of 2-[[3-(azidomethyl)cyclobutyl]amino]-N-[6-(dimethylamino)pyridazin-3-yl]-6-(methylamino)pyridine-3-carboxamide

To a solution of [3-[[3-[[6-(dimethylamino)pyridazin-3-yl]carbamoyl]-6-(methylamino)-2-pyridyl]amino]cyclobutyl]methyl 4-methylbenzenesulfonate (230 mg, 438 μmol) in DMF (5 mL) was added NaN₃ (43 mg, 656 μmol). The mixture was stirred at 80° C. for 1 h. The reaction mixture was poured into ice water (15 mL), and then sat. NaOH was added until pH >9. The mixture was extracted with ethyl acetate (10 mL×3). The combined organic layers were washed with brine (15 mL), dried over Na₂SO₄, filtered, and concentrated under reduced pressure to provide the desired product (126 mg, 63% yield) as a yellow solid. ¹HNMR (400 MHz, CDCl₃) (8.60 (d, J=5.6 Hz, 1H), 8.09 (d, J=9.6 Hz, 1H), 7.54 (d, J=8.4 Hz, 1H), 6.83 (d, J=9.6 Hz, 1H), 5.63 (d, J=8.4 Hz, 1H), 4.67 (s, 1H), 4.61-4.51 (m, 1H), 3.36 (d, J=7.6 Hz, 2H), 3.08 (s, 6H), 2.89 (s, 3H), 2.30-2.26 (m, 1H), 2.26-2.18 (m, 2H), 2.16-2.06 (m, 2H). MS (ESI) m/z=397.4 [M+H]⁺.

### Step 7. Synthesis of 2-((3-((4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)methyl)cyclobutyl)amino)-N-(6-(dimethylamino)pyridazin-3-yl)-6-(methylamino)nicotinamide

A mixture of 2-((3-(azidomethyl)cyclobutyl)amino)-N-(6-(dimethylamino)pyridazin-3-yl)-6-(methylamino)nicotinamide (10 mg, 25.22 μmol), 6-acetyl-8-cyclopentyl-5-methyl-2-((5-(4-(prop-2-yn-1-yl)piperazin-1-yl)pyridin-2-yl)amino)pyrido[2,3-d]pyrimidin-7(8H)-one (14.70 mg, 30.27 μmol), CuSO₄ (6.04 mg, 37.84 μmol) and sodium ascorbate (7.50 mg, 37.84 μmol) in water (1 mL) and THF (2 mL) was stirred at rt for 4 h. The reaction mixture was poured into water (20 mL) and extracted with ethyl acetate (3×10 mL). The combined organic layers were washed with saturated brine (15 mL), dried over anhydrous sodium sulfate, filtered, and evaporated under reduced pressure. The resulting residue was purified by reverse-phase chromatography to give the desired product (0.7 mg, 3% yield) as a yellow solid. MS (ESI) m/z=882.7 [M+H]⁺.

### Example 261. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(6-(dimethylamino)pyridazin-3-yl)-6-(methylamino)nicotinamide (CPD-203)

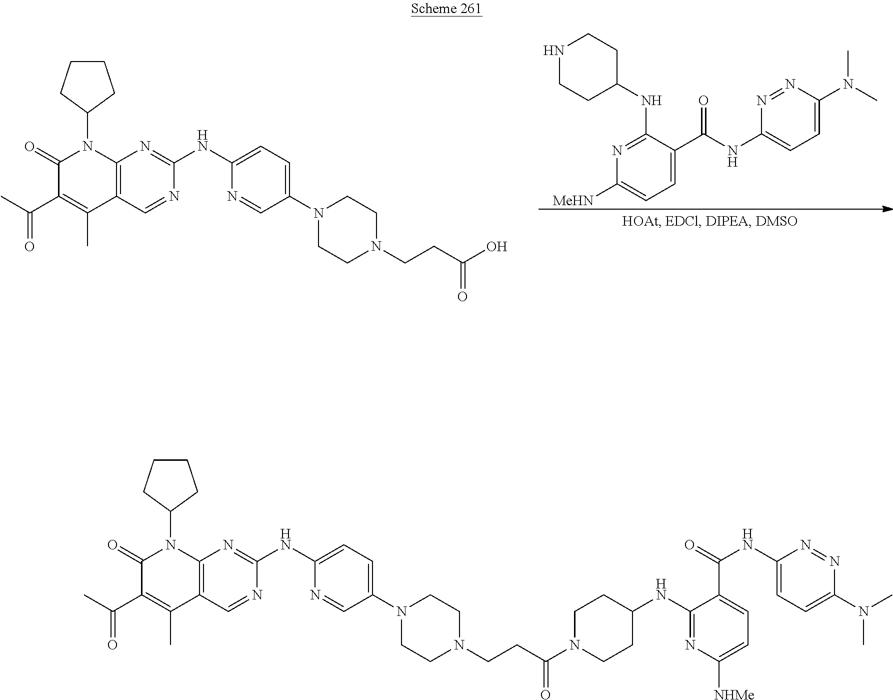 [SMILES: CC(=O)c1c(C)c2cnc(Nc3ccc(N4CCN(CCC(=O)O)CC4)cn3)nc2n(C2CCCC2)c1=O ; CNc1ccc(C(=O)Nc2ccc(N(C)C)nn2)c(NC2CCNCC2)n1 ; CNc1ccc(C(=O)Nc2ccc(N(C)C)nn2)c(NC2CCN(C(=O)CCN3CCN(c4ccc(Nc5ncc6c(C)c(C(C)=O)c(=O)n(C7CCCC7)c6n5)nc4)CC3)CC2)n1]

CPD-201 was synthesized following the standard procedures for preparing CPD-143 (5.4 mg, 11% yield) as a yellow solid. MS (ESI) m/z=872.9 [M+H]⁺.

### Example 262. 2-(((trans-4-(3-(4-(6-((6-acetyl-8-cyclopentyl-S-methyl-7-oxo-7,8-dihydropyrido[2,3-d]pyrimidin-2-yl)amino)pyridin-3-yl)piperazin-1-yl)-3-oxopropyl)cyclohexyl)methyl)amino)-N-(6-(dimethylamino)pyridazin-3-yl)-6-(methylamino)nicotinamide (CPD-204)

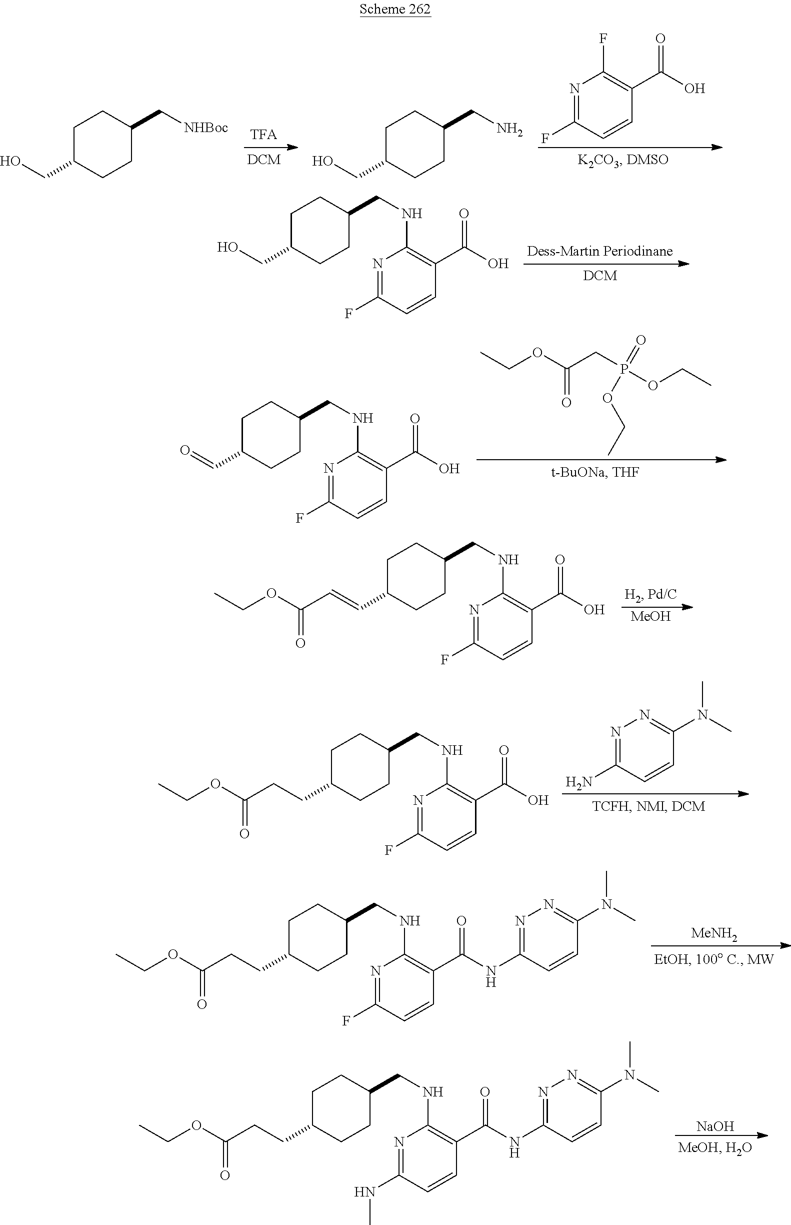

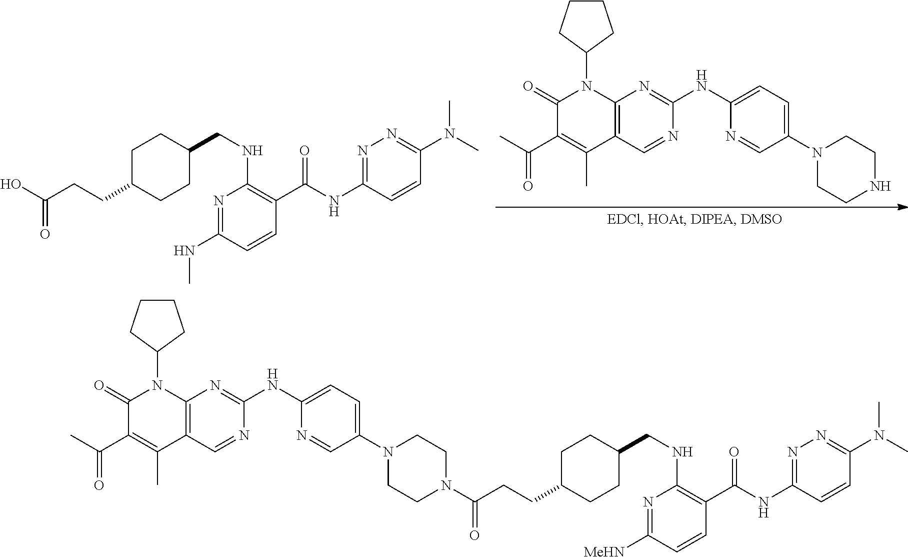

### Step 1. Synthesis of (trans-4-(aminomethyl)cyclohexyl)methanol

A solution of tert-butyl((trans-4-(hydroxymethyl)cyclohexyl)methyl)carbamate (500 mg, 2.05 mmol) in dichloromethane (8 mL) and TFA (4 mL) was stirred at rt for 1 h. The reaction mixture was concentrated under reduced pressure. The residue was diluted with water (30 mL), alkalized with saturated sodium bicarbonate solution, and extracted with ethyl acetate (3×30 mL). The combined organic layers were washed with saturated brine (45 mL), dried over anhydrous sodium sulfate, filtered, and evaporated under reduced pressure. The resulting residue was purified by silica gel chromatography to give the desired product (290 mg, 98% yield) as a yellow oil. MS (ESI) m/z=144.2 [M+H]⁺.

### Step 2. Synthesis of 6-fluoro-2-(((trans-4-(hydroxymethyl)cyclohexyl)methyl)amino)nicotinic acid

To a solution of (trans-4-(aminomethyl)cyclohexyl)methanol (290 mg, 2.02 mmol) in DMSO (10 mL) were added 2,6-difluoronicotinic acid (386.55 mg, 2.43 mmol) and K₂CO₃ (559.69 mg, 4.05 mmol) at rt. The reaction mixture was stirred at 100° C. for 16 h. The mixture was cooled to rt, poured into water (50 mL), and extracted with ethyl acetate (3×30 mL). The combined organic layers were washed with saturated brine, dried over anhydrous sodium sulfate, filtered, and evaporated under reduced pressure. The resulting residue was purified by reverse-phase chromatography to give the desired product (310 mg, 54% yield) as a pale-white solid. MS (ESI) m/z=283.5 [M+H]⁺.

### Step 3. Synthesis of 6-fluoro-2-(((trans-4-formylcyclohexyl)methyl)amino)nicotinic acid

To a suspension of 6-fluoro-2-(((trans-4-(hydroxymethyl)cyclohexyl)methyl)amino)nicotinic acid (300 mg, 1.06 mmol) in dichloromethane (10 mL) was added Dess-Martin periodinane (495.63 mg, 1.17 mmol) at 0° C. The reaction mixture was stirred at 0° C. for 2 h. The reaction mixture was diluted with dichloromethane (10 mL) and filtered through a celite. The filtrate was concentrated and purified by silica gel chromatography to give the desired product (257 mg, 86% yield) as a pale-white solid. MS (ESI) m/z=281.3 [M+H]⁺.

### Step 4. Synthesis of 2-(((trans-4-((E)-3-ethoxy-3-oxoprop-1-en-1-yl)cyclohexyl)methyl)amino)-6-fluoronicotinic acid

To a solution of 6-fluoro-2-(((trans-4-formylcyclohexyl)methyl)amino)nicotinic acid (257 mg, 916.89 μmol) in THF (10 mL) was added t-BuONa (176.23 mg, 1.83 mmol) at 0° C. After stirring at 0° C. for 0.5 h, ethyl 2-diethoxyphosphorylacetate (308.34 mg, 1.38 mmol) was added to the above solution. The mixture was stirred at the same temperature for another 2 h. The reaction mixture was diluted with ice water and extracted with ethyl acetate (3×20 mL). The combined organic layers were washed with saturated brine (30 mL), dried over anhydrous sodium sulfate, filtered, and evaporated under reduced pressure. The resulting residue was purified by silica gel chromatography to give the desired product (280 mg, 87% yield) as a pale-white solid. MS (ESI) m/z=351.5 [M+H]⁺.

### Step 5. Synthesis of 2-(((trans-4-(3-ethoxy-3-oxopropyl)cyclohexyl)methyl)amino)-6-fluoronicotinic acid

To a solution of 2-(((trans-4-((E)-3-ethoxy-3-oxoprop-1-en-1-yl)cyclohexyl)methyl)amino)-6-fluoronicotinic acid (280 mg, 799.12 μmol) in MeOH (10 mL) was added Pd/C (5%, 20 mg). The mixture was stirred at rt under hydrogen atmosphere for 2 h. The reaction mixture was filtered and concentrated under vacuum. The residue was purified by silica gel column chromatography to give the desired product (180 mg, 64% yield) as a white solid. MS (ESI) m/z=353.5 [M+H]⁺.

### Step 6. Synthesis of ethyl 3-(trans-4-(((3-((6-(dimethylamino)pyridazin-3-yl)carbamoyl)-6-fluoropyridin-2-yl)amino)methyl)cyclohexyl)propanoate

To a solution of 2-(((trans-4-(3-ethoxy-3-oxopropyl)cyclohexyl)methyl)amino)-6-fluoronicotinic acid (90 mg, 255.39 mol) in THF (5 mL) were added N³,N³-dimethylpyridazine-3,6-diamine (35.29 mg, 255.39 μmol), BOP (338.86 mg, 766.18 μmol) and TEA (129.22 mg, 1.28 mmol). The reaction mixture was stirred at 50° C. for 4 h. The reaction mixture was poured into water (50 mL) and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography to give the desired product (70 mg, 58% yield) as a white solid. MS (ESI) m/z=473.7 [M+H]⁺.

### Step 7. Synthesis of ethyl 3-(trans-4-(((3-((6-(dimethylamino)pyridazin-3-yl)carbamoyl)-6-(methylamino)pyridin-2-yl)amino)methyl)cyclohexyl)propanoate

A solution of ethyl 3-(trans-4-(((3-((6-(dimethylamino)pyridazin-3-yl)carbamoyl)-6-fluoropyridin-2-yl)amino)methyl)cyclohexyl)propanoate (70 mg, 148.13 μmol) in MeNH₂ solution (30% in EtOH, 2 mL) was stirred at 100° C. under microwave irradiation for 10 min. The reaction mixture was poured into water (30 mL) and extracted with ethyl acetate (3×10 mL). The combined organic layers were washed with saturated brine (15 mL), dried over anhydrous sodium sulfate, filtered, and evaporated under reduced pressure. The resulting residue was purified by silica gel chromatography to give the desired product (65 mg, 91% yield) as a yellow solid. MS (ESI) m/z=484.6 [M+H]⁺.

### Step 8. Synthesis of 3-(trans-4-(((3-((6-(dimethylamino)pyridazin-3-yl)carbamoyl)-6-(methylamino)pyridin-2-yl)amino)methyl)cyclohexyl)propanoic acid

To a solution of ethyl 3-(trans-4-(((3-((6-(dimethylamino)pyridazin-3-yl)carbamoyl)-6-(methylamino)pyridin-2-yl)amino)methyl)cyclohexyl)propanoate (65 mg, 134.41 μmol) in water (2 mL) and MeOH (2 mL) was added NaOH (53.76 mg, 1.34 mmol). The reaction mixture was stirred at 80° C. for 1 h. The reaction mixture was cooled to rt and poured into water (30 mL), acidified with aq. HCl (1 M), and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography to give the desired product (48 mg, 78% yield) as a pale-white solid. MS (ESI) m/z=456.6 [M+H]⁺.

### Step 9. Synthesis of 2-(((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-3-oxopropyl)cyclohexyl)methyl)amino)-N-(6-(dimethylamino)pyridazin-3-yl)-6-(methylamino)nicotinamide

To a mixture of 3-(trans-4-(((3-((6-(dimethylamino)pyridazin-3-yl)carbamoyl)-6-(methylamino)pyridin-2-yl)amino)methyl)cyclohexyl)propanoic acid (48 mg, 105.37 μmol), EDCI (30.30 mg, 158.05 μmol) and HOAt (21.51 mg, 158.05 μmol) in DMSO (1 mL) were added DIPEA (68.09 mg, 526.83 μmol, 87.07 μL) and 6-acetyl-8-cyclopentyl-5-methyl-2-((5-(piperazin-1-yl)pyridin-2-yl)amino)pyrido[2,3-d]pyrimidin-7(8H)-one (56.59 mg, 126.44 μmol). The mixture was stirred at rt for 16 h, before it was purified by prep-HPLC to provide the desired product (1.4 mg, 12% yield) as a yellow solid. MS (ESI) m/z=885.7 [M+H]⁺.

### Example 263. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-(isopropylamino)nicotinamide (CPD-205)

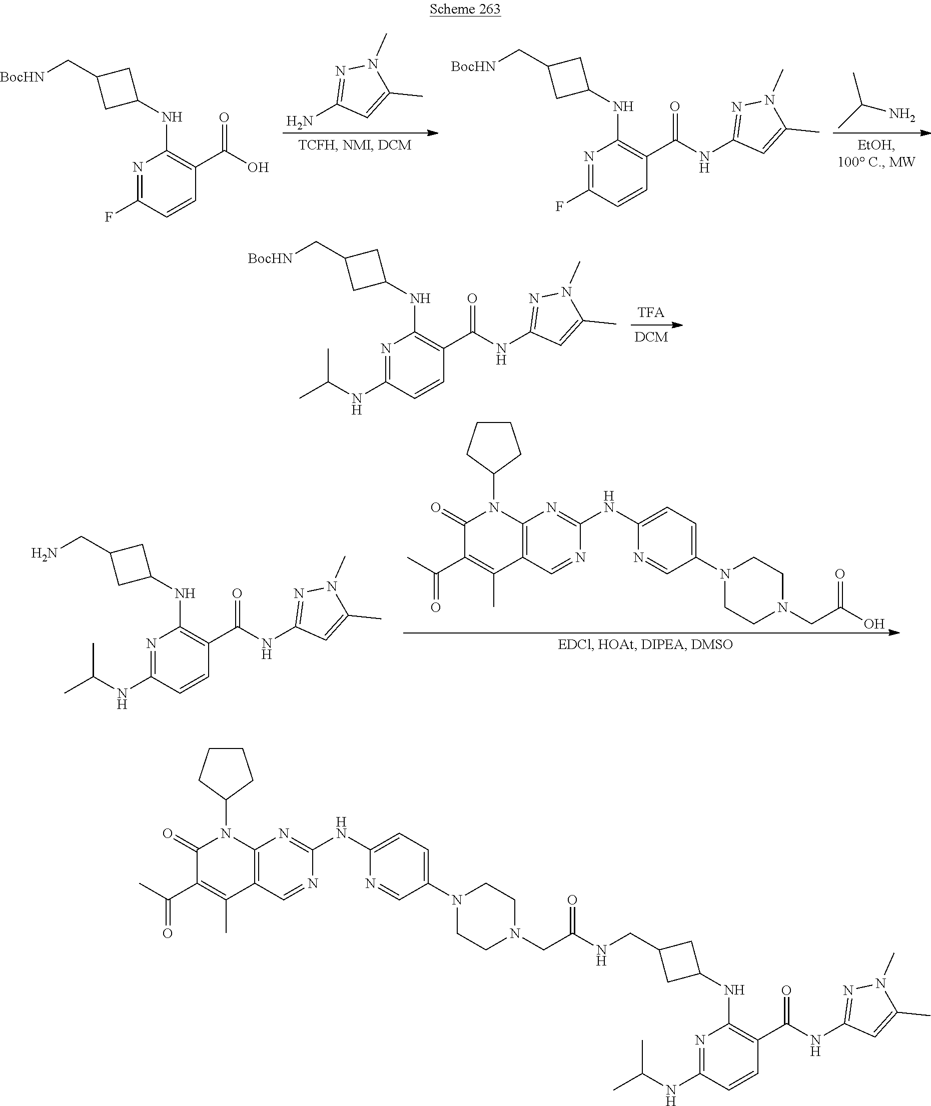

CPD-205 was synthesized following the standard procedures for preparing CPD-144 (4.4 mg, 8% yield over 4 steps) as a yellow solid. MS (ESI) m/z=859.8 [M+H]⁺.

### Example 264. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-((methyl-d₃)amino)nicotinamide (CPD-206)

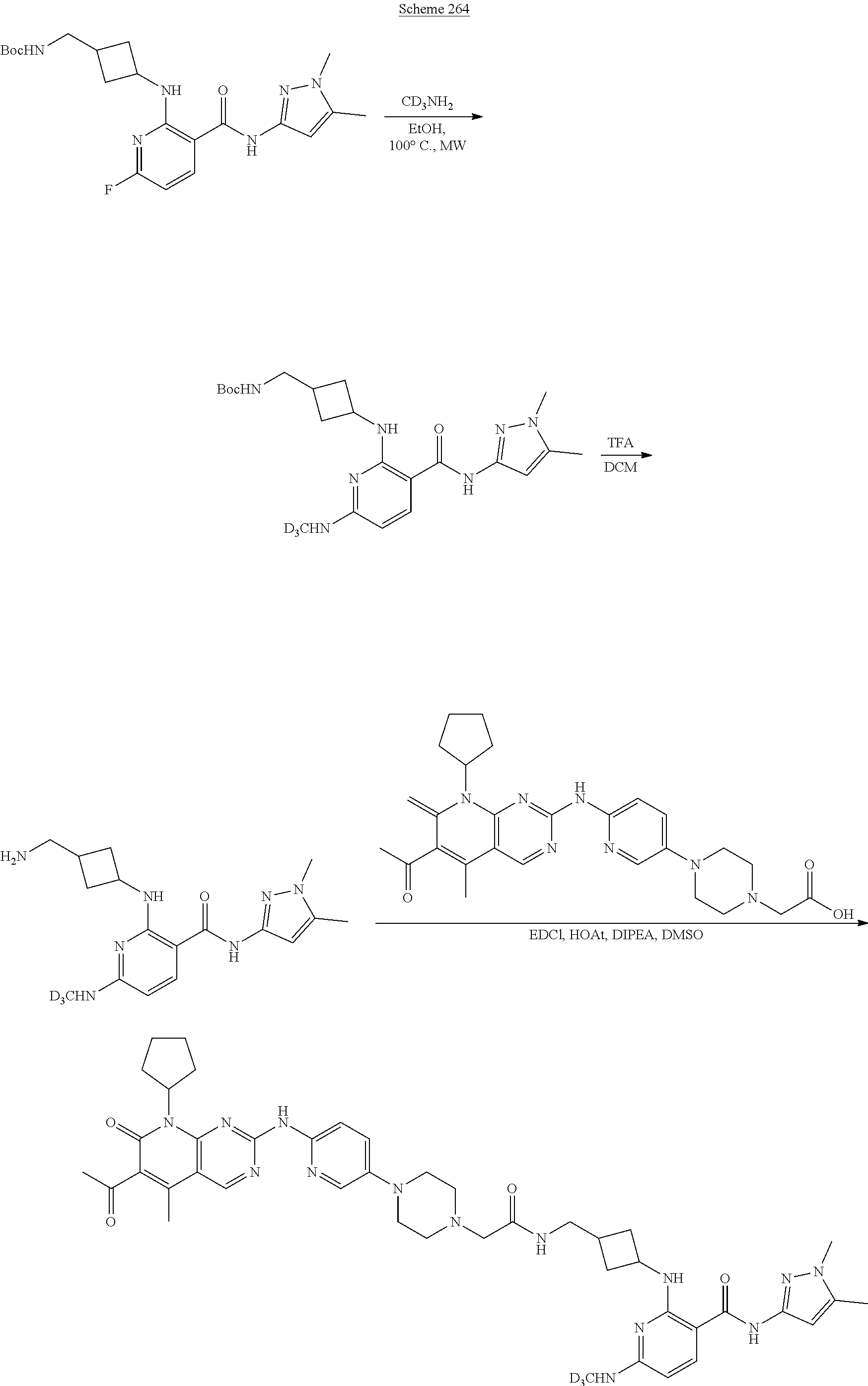

CPD-206 was synthesized following the standard procedures for preparing CPD-144 (5.3 mg, 12% yield over 3 steps) as a yellow solid. MS (ESI) m/z=834.8 [M+H]⁺.

### Example 265. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-methylnicotinamide (CPD-207)

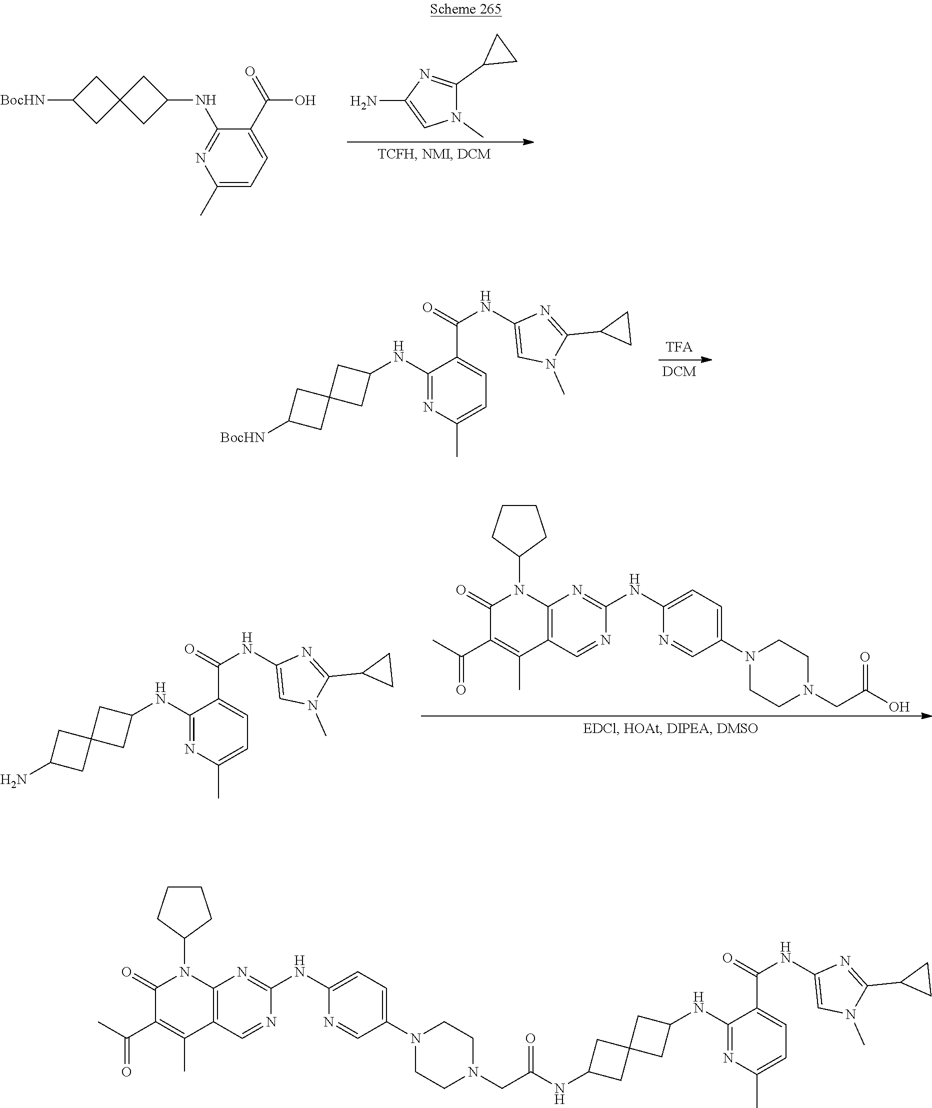

CPD-207 was synthesized following the standard procedures for preparing CPD-144 (1.1 mg, 2% yield over 3 steps) as a yellow solid. MS (ESI) m/z=868.7 [M+H]⁺.

### Example 266. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)-2-azaspiro[3.3]heptan-6-yl)amino)-N-(6-(dimethylamino)pyridazin-3-yl)-6-(methylamino)nicotinamide (CPD-208)

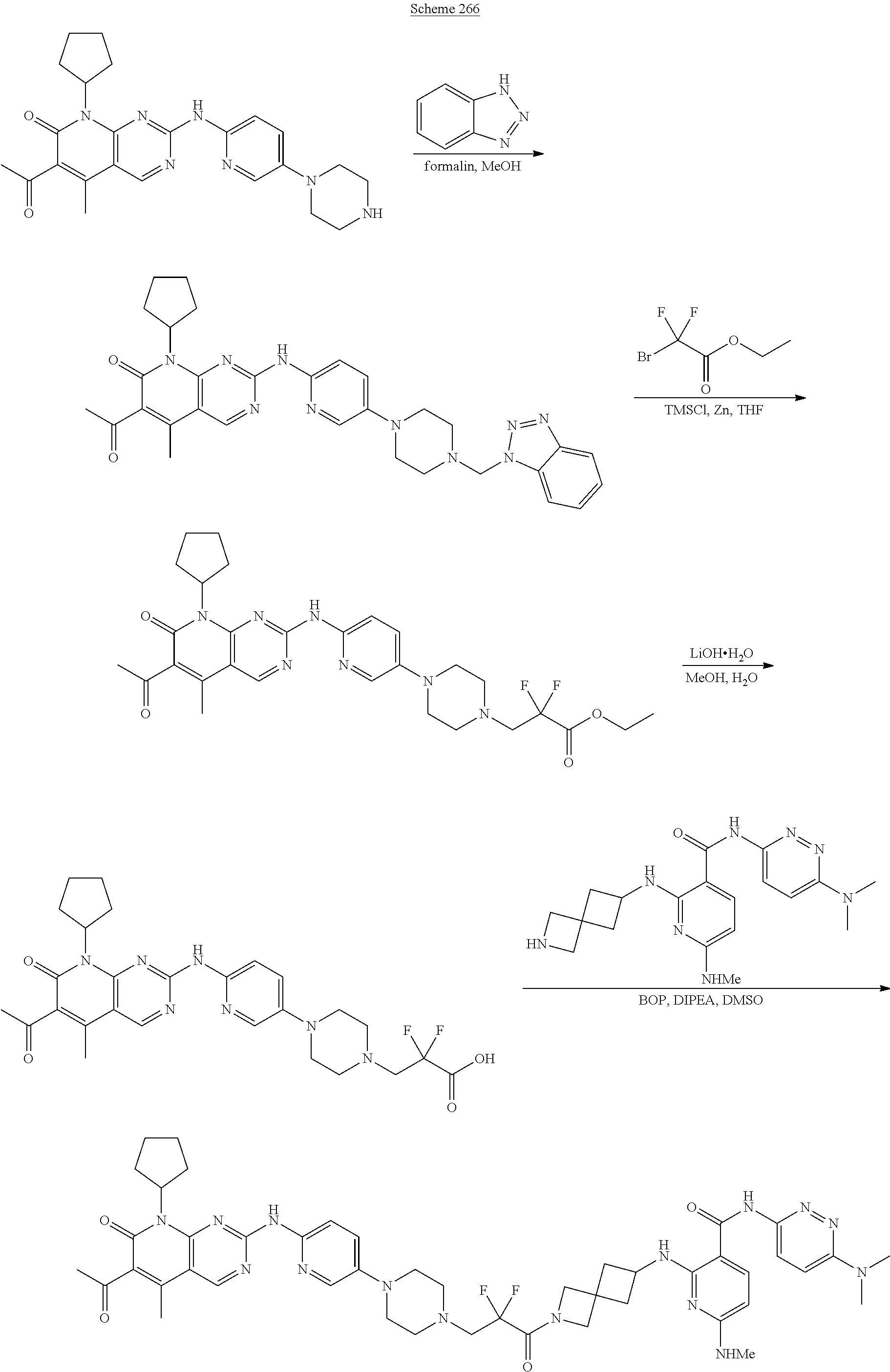

### Step 1. Synthesis of 2-((5-(4-((1H-benzo[d][1,2,3]triazol-1-yl)methyl)piperazin-1-yl)pyridin-2-yl)amino)-6-acetyl-8-cyclopentyl-5-methylpyrido[2,3-d]pyrimidin-7(8H)-one

To a solution of 6-acetyl-8-cyclopentyl-5-methyl-2-((5-(piperazin-1-yl)pyridin-2-yl)amino)pyrido[2,3-d]pyrimidin-7(8H)-one (300 mg, 0.67 mmol) in MeOH (20 mL) were added 1H-benzo[d][1,2,3]triazole (160 mg, 1.34 mmol) and formalin (37% in water, 2 mL). After the mixture was stirred at rt for 2 h, it was filtered and evaporated under reduced pressure to give the crude product (320 mg, 83% yield) as a yellow solid.

### Step 2. Synthesis of ethyl 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoate

To a solution of zinc (102 mg, 1.56 mmol) in THE (25 mL) was added TMSCl (280 mg, 2.59 mmol) at rt under nitrogen. The reaction mixture was stirred at the same temperature for 30 min, then ethyl 2-bromo-2,2-difluoroacetate (210 mg, 1.04 mmol) was added dropwise. The reaction mixture was stirred at rt for another 30 min, before 2-((5-(4-((1H-benzo[d][1,2,3]triazol-1-yl)methyl)piperazin-1-yl)pyridin-2-yl)amino)-6-acetyl-8-cyclopentyl-5-methylpyrido[2,3-d]pyrimidin-7(8H)-one (300 mg, 0.52 mmol) was added. The resulting mixture was stirred at rt for 3 h, then it was quenched with water (30 mL) slowly and extracted with ethyl acetate (2×50 mL). The combined organic layers were washed with saturated brine (30 mL), dried over anhydrous sodium sulfate, filtered, and evaporated under reduced pressure. The residue was purified by silica gel chromatography to provide the desired product (250 mg, 83% yield) as a yellow solid. MS (ESI) m/z=584.5 [M+H]⁺.

### Step 3. Synthesis of 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoic acid

To a solution of ethyl 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoate (250 mg, 0.43 mmol) in MeOH (20 mL) and water (5 mL) was added LiOH·H₂O (180 mg, 4.28 mmol). The mixture was stirred at 50° C. for 4 h. After cooling down to rt, the solution was diluted with water (30 mL) and the pH was adjusted to 5-6 with HCl solution (1 M). The aqueous phase was extracted with ethyl acetate (3×50 mL). The combined organic layers were washed with saturated brine (30 mL), dried over anhydrous sodium sulfate, filtered, and evaporated under reduced pressure. The resulting residue was purified by reverse-phase chromatography to provide the desired product (200 mg, 84% yield) as a yellow solid. MS (ESI) m/z=556.5 [M+H]⁺.

### Step 4. Synthesis of 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)-2-azaspiro[3.3]heptan-6-yl)amino)-N-(6-(dimethylamino)pyridazin-3-yl)-6-(methylamino)nicotinamide

To a solution of 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoic acid (65 mg, 0.12 mmol) in DMSO (3 mL) were added 2-((2-azaspiro[3.3]heptan-6-yl)amino)-N-(6-(dimethylamino)pyridazin-3-yl)-6-(methylamino)nicotinamide (45 mg, 0.12 mmol), DIPEA (76 mg, 0.59 mmol) and BOP (104 mg, 0.24 mmol). The mixture was stirred at rt for 2 h, before it was purified by prep-HPLC to afford the desired product (35 mg, 32% yield) as a yellow solid. MS (ESI) m/z=920.6 [M+H]⁺.

### Example 267. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-methylnicotinamide (CPD-209)

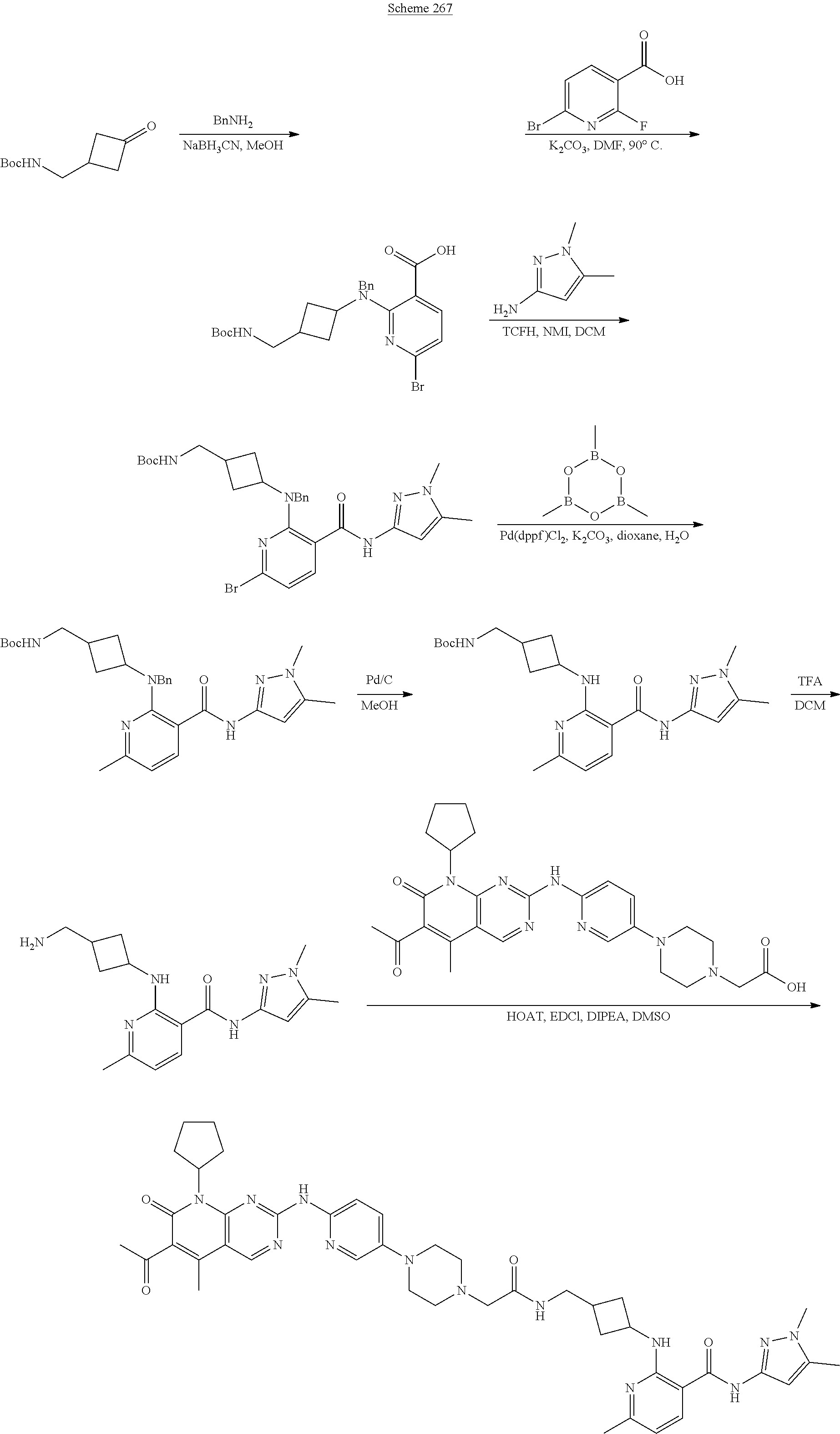

### Step 1. Synthesis of tert-butyl ((3-(benzylamino)cyclobutyl)methyl)carbamate

To a solution of tert-butyl ((3-oxocyclobutyl)methyl)carbamate (500 mg, 2.51 mmol) in MeOH (20 mL) were added phenylmethanamine (269 mg, 2.51 mmol) and NaBH₃CN (790 mg, 12.55 mmol). After the mixture was stirred rt for 4 h, it was quenched with water (30 mL) and extracted with ethyl acetate (2×50 mL). The combined organic layers were washed with saturated brine (30 mL), dried over anhydrous sodium sulfate, filtered, and evaporated under reduced pressure. The resulting residue was purified by silica gel chromatography to provide the desired product (600 mg, 82% yield) as a colorless oil. MS (ESI) m/z=291.4 [M+H]⁺.

### Step 2. Synthesis of 2-(benzyl(3-(((tert-butoxycarbonyl)amino)methyl)cyclobutyl)amino)-6-bromonicotinic acid

To a solution of tert-butyl ((3-(benzylamino)cyclobutyl)methyl)carbamate (600 mg, 2.07 mmol) in DMF (15 mL) were added 6-bromo-2-fluoronicotinic acid (455 mg, 2.07 mmol) and K₂CO₃ (1.43 g, 10.33 mmol). The mixture was stirred at 90° C. for 6 h, After cooling down to rt, the solution was diluted with water (30 mL) and the pH was adjusted to 56 with HCl solution (1 M). The aqueous phase was extracted with ethyl acetate (3×50 mL). The combined organic layers were washed with saturated brine (50 mL), dried over anhydrous sodium sulfate, filtered, and evaporated under reduced pressure. The resulting residue was purified by reverse-phase chromatography to provide the desired product (600 mg, 59% yield) as a light-yellow solid. MS (ESI) m/z=490.3[M+H]⁺.

### Step 3. Synthesis of tert-butyl ((3-(benzyl(6-bromo-3-((1,5-dimethyl-1H-pyrazol-3-yl)carbamoyl)pyridin-2-yl)amino)cyclobutyl)methyl)carbamate

To a solution of 2-(benzyl(3-(((tert-butoxycarbonyl)amino)methyl)cyclobutyl)amino)-6-bromonicotinic acid (600 mg, 1.22 mmol) in dichloromethane (15 mL) were added 1,5-dimethyl-1H-pyrazol-3-amine (136 mg, 1.22 mmol), TCFH (686 mg, 2.45 mmol) and NMI (502 mg, 6.12 mmol). The mixture was stirred at rt for 3 h, before it was purified by silica gel chromatography to provide the desired product (600 mg, 84% yield) as a brown solid. MS (ESI) m/z=585.5 [M+H]⁺.

### Step 4. Synthesis of tert-butyl ((3-(benzyl(3-((1,5-dimethyl-1H-pyrazol-3-yl)carbamoyl)-6-methylpyridin-2-yl)amino)cyclobutyl)methyl)carbamate

To a solution of tert-butyl ((3-(benzyl(6-bromo-3-((1,5-dimethyl-1H-pyrazol-3-yl)carbamoyl)pyridin-2-yl)amino)cyclobutyl)methyl)carbamate (70 mg, 0.12 mmol) in dioxane (6 mL) and water (2 mL) were added 2,4,6-trimethyl-1,3,5,2,4,6-trioxatriborinane (15 mg, 0.12 mmol), K₂CO₃ (83 mg, 0.60 mmol) and Pd(dppf)Cl₂ (18 mg, 0.024 mmol) under argon gas atmosphere. The resulting mixture was stirred at 90° C. for 3 h. After cooling down to rt, the solution was diluted with water (20 mL) and extracted with ethyl acetate (3×30 mL). The combined organic layers were washed with saturated brine (20 mL), dried over anhydrous sodium sulfate, filtered, and evaporated under reduced pressure. The resulting residue was purified by silica gel chromatography to provide the desired product (50 mg, 80% yield) as a light-yellow solid. MS (ESI) m/z=519.8 [M+H]⁺.

### Step 5. Synthesis of tert-butyl ((3-((3-((1,5-dimethyl-1H-pyrazol-3-yl)carbamoyl)-6-methylpyridin-2-yl)amino)cyclobutyl)methyl)carbamate

To a solution of tert-butyl ((3-(benzyl(3-((1,5-dimethyl-1H-pyrazol-3-yl)carbamoyl)-6-methylpyridin-2-yl)amino)cyclobutyl)methyl)carbamate (50 mg, 0.096 mmol) in MeOH (10 mL) was added 5% Pd/C (35 mg). The mixture was stirred at rt under hydrogen atmosphere for 5 h. The mixture was filtered and concentrated to provide the desired product (30 mg, 73% yield) as a colorless solid. MS (ESI) m/z=429.4[M+H]⁺.

The remaining steps were performed following the standard procedures for preparing CPD-144 to provide the desired product (10 mg, 17% yield over 2 steps) as a yellow solid. MS (ESI) m/z=816.7 [M+H]⁺.

### Example 268. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-ethylnicotinamide (CPD-210)

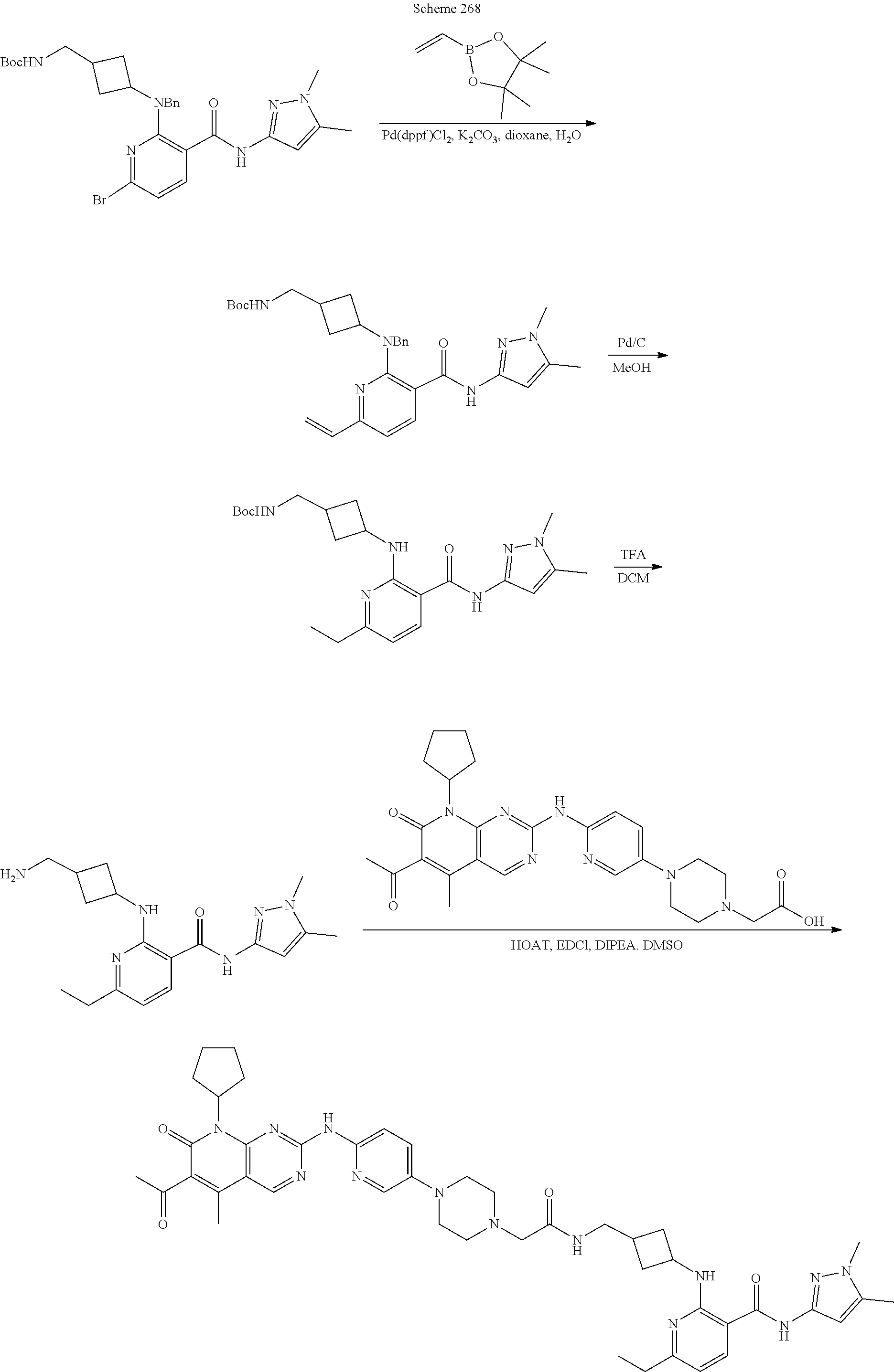

CPD-210 was synthesized following the standard procedures for preparing CPD-209 (17 mg, 17% yield over 4 steps) as a yellow solid. MS (ESI) m/z=830.8 [M+H]⁺.

### Example 269. 2-((1-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)azetidin-3-yl)amino)-N-(6-(dimethylamino)pyridazin-3-yl)-6-(methylamino)nicotinamide (CPD-211)

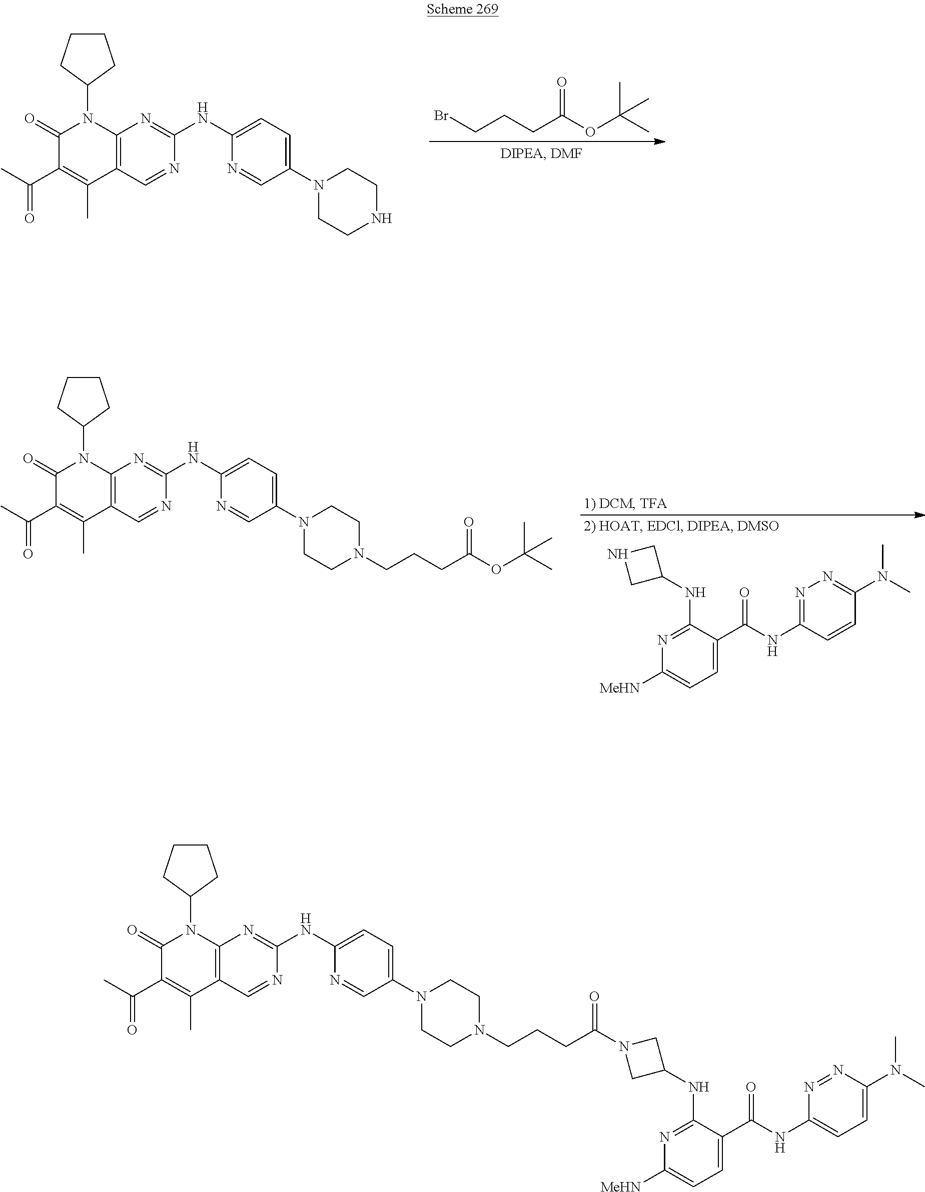 [SMILES: CC(=O)c1c(C)c2cnc(Nc3ccc(N4CCNCC4)cn3)nc2n(C2CCCC2)c1=O ; CC(C)(C)OC(=O)CCCBr ; CC(=O)c1c(C)c2cnc(Nc3ccc(N4CCN(CCCC(=O)OC(C)(C)C)CC4)cn3)nc2n(C2CCCC2)c1=O ; CNc1ccc(C(=O)Nc2ccc(N(C)C)nn2)c(NC2CNC2)n1 ; CNc1ccc(C(=O)Nc2ccc(N(C)C)nn2)c(NC2CN(C(=O)CCCN3CCN(c4ccc(Nc5ncc6c(C)c(C(C)=O)c(=O)n(C7CCCC7)c6n5)nc4)CC3)C2)n1]

### Step 1. Synthesis of tert-butyl 4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoate

To a solution of 6-acetyl-8-cyclopentyl-5-methyl-2-((5-(piperazin-1-yl)pyridin-2-yl)amino)pyrido[2,3-d]pyrimidin-7(8H)-one (300 mg, 0.67 mmol) in DMF (10 mL) were added tert-butyl 4-bromobutanoate (150 mg, 0.67 mmol) and DIPEA (433 mg, 3.35 mmol). The mixture was stirred at 90° C. for 6 h. After cooling down to rt, the solution was diluted with water (50 mL) and extracted with ethyl acetate (3×50 mL). The combined organic layers were washed with saturated brine (50 mL), dried over anhydrous sodium sulfate, filtered, and evaporated under reduced pressure. The resulting residue was purified by silica gel chromatography to provide the desired product (350 mg, 89% yield) as a yellow solid. MS(ESI) m/z=590.8[M+H]⁺.

The remaining steps were performed following the standard procedures for preparing CPD-144 to provide the desired product (10 mg, 25% yield over 2 steps) as a yellow solid. MS (ESI) m/z=858.8 [M+H]⁺.

### Example 270. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-6-isopropylnicotinamide (CPD-212)

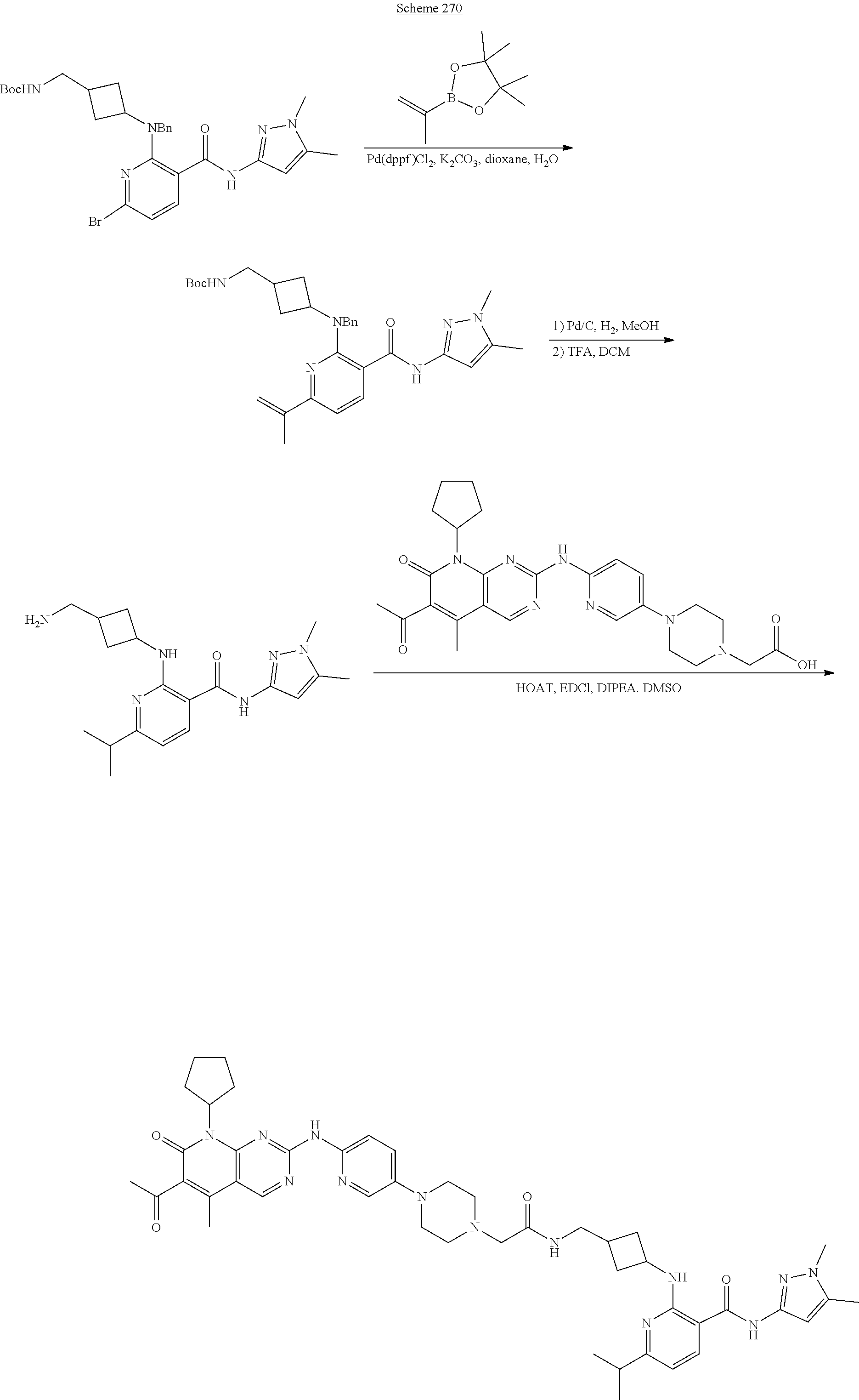 [SMILES: Cc1cc(NC(=O)c2ccc(Br)nc2N(Cc2ccccc2)C2CC(CNC(=O)OC(C)(C)C)C2)nn1C ; C=C(C)B1OC(C)(C)C(C)(C)O1 ; C=C(C)c1ccc(C(=O)Nc2cc(C)n(C)n2)c(N(Cc2ccccc2)C2CC(CNC(=O)OC(C)(C)C)C2)n1 ; Cc1cc(NC(=O)c2ccc(C(C)C)nc2NC2CC(CN)C2)nn1C ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCC5CC(Nc6nc(C(C)C)ccc6C(=O)Nc6cc(C)n(C)n6)C5)CC4)cn3)nc2n(C2CCCC2)c1=O]

CPD-212 was synthesized following the standard procedures for preparing CPD-209 (8.0 mg, 8% yield over 4 steps) as a yellow solid. MS (ESI) m/z=844.8 [M+H]⁺.

### Example 271. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-cyclopropyl-N-(1,5-dimethyl-1H-pyrazol-3-yl)nicotinamide (CPD-213)

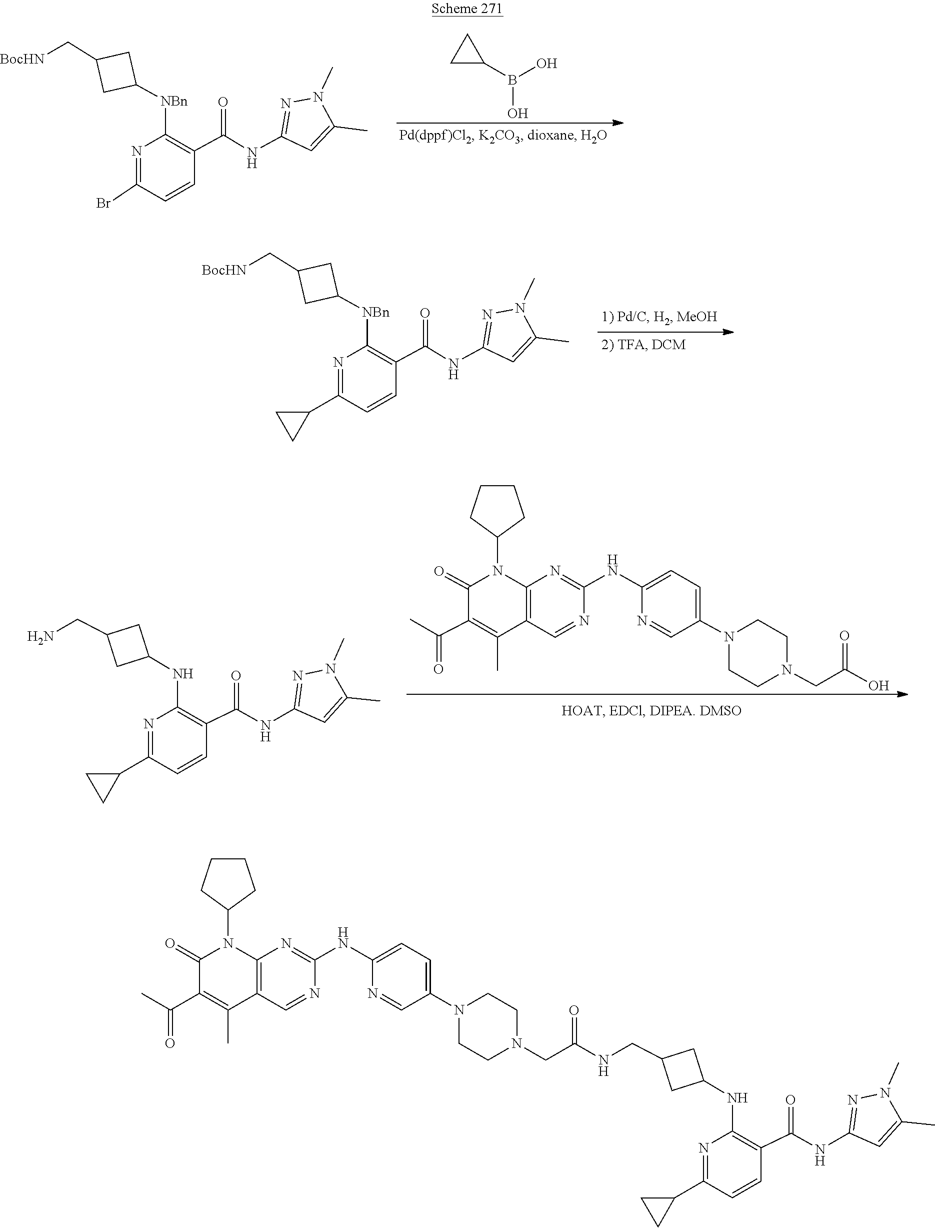 [SMILES: Cc1cc(NC(=O)c2ccc(Br)nc2N(Cc2ccccc2)C2CC(CNC(=O)OC(C)(C)C)C2)nn1C ; OB(O)C1CC1 ; Cc1cc(NC(=O)c2ccc(C3CC3)nc2N(Cc2ccccc2)C2CC(CNC(=O)OC(C)(C)C)C2)nn1C ; Cc1cc(NC(=O)c2ccc(C3CC3)nc2NC2CC(CN)C2)nn1C ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCC5CC(Nc6nc(C7CC7)ccc6C(=O)Nc6cc(C)n(C)n6)C5)CC4)cn3)nc2n(C2CCCC2)c1=O]

CPD-213 was synthesized following the standard procedures for preparing CPD-209 (5.0 mg, 4% yield over 4 steps) as a yellow solid. MS (ESI) m/z=842.7 [M+H]⁺.

### Example 272. 2-(((4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-oxoethoxy)cyclohexyl)methyl)amino)-N-(6-(dimethylamino)pyridazin-3-yl)-6-(methylamino)nicotinamide (CPD-214)

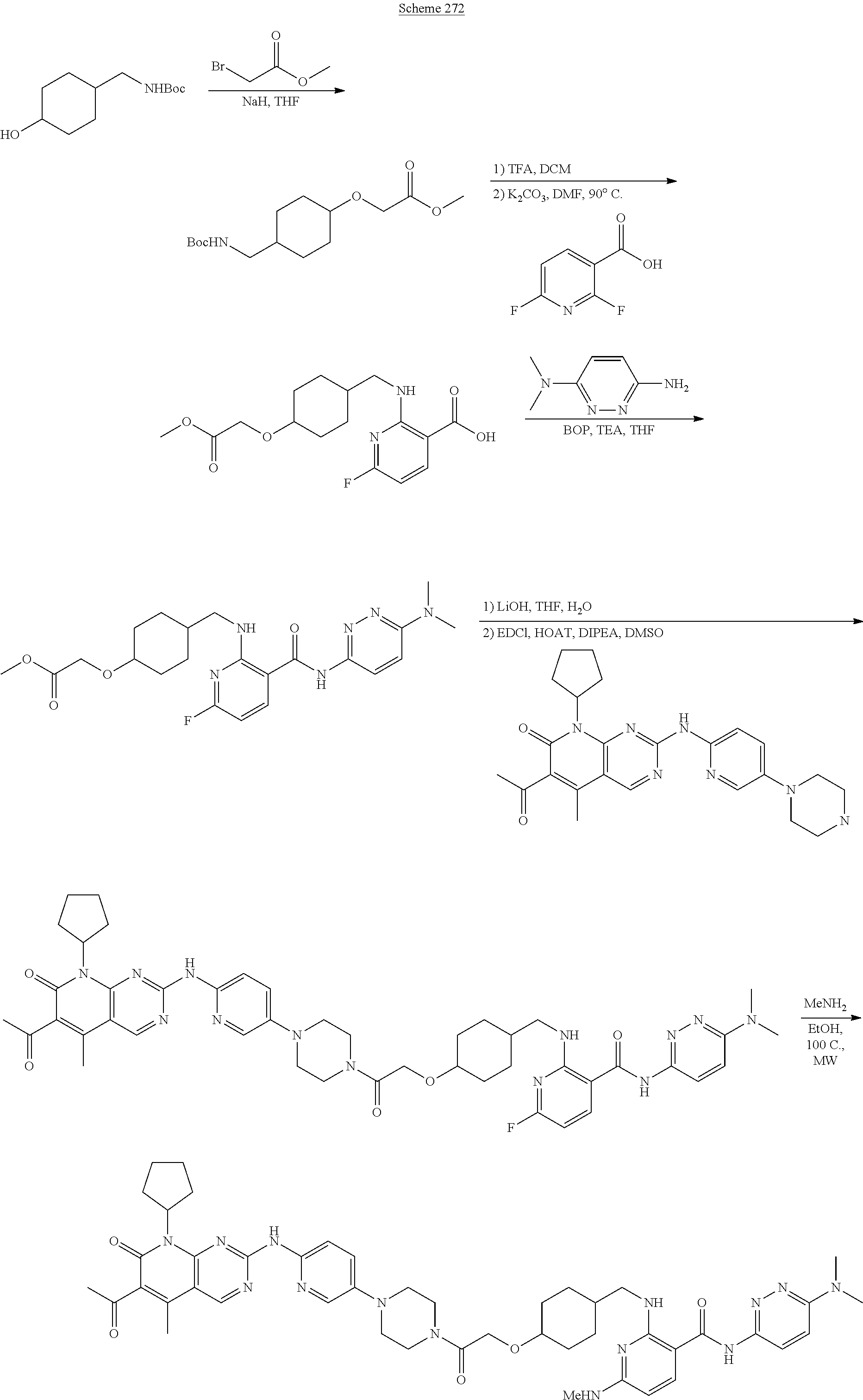

CPD-214 was synthesized following the standard procedures for preparing CPD-189 (1.7 mg, 3% yield over 7 steps) as a yellow solid. MS (ESI) m/z=887.7 [M+H]⁺.

### Example 273. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-cyclopropylisoxazol-3-yl)-6-(methylamino)nicotinamide (CPD-215)

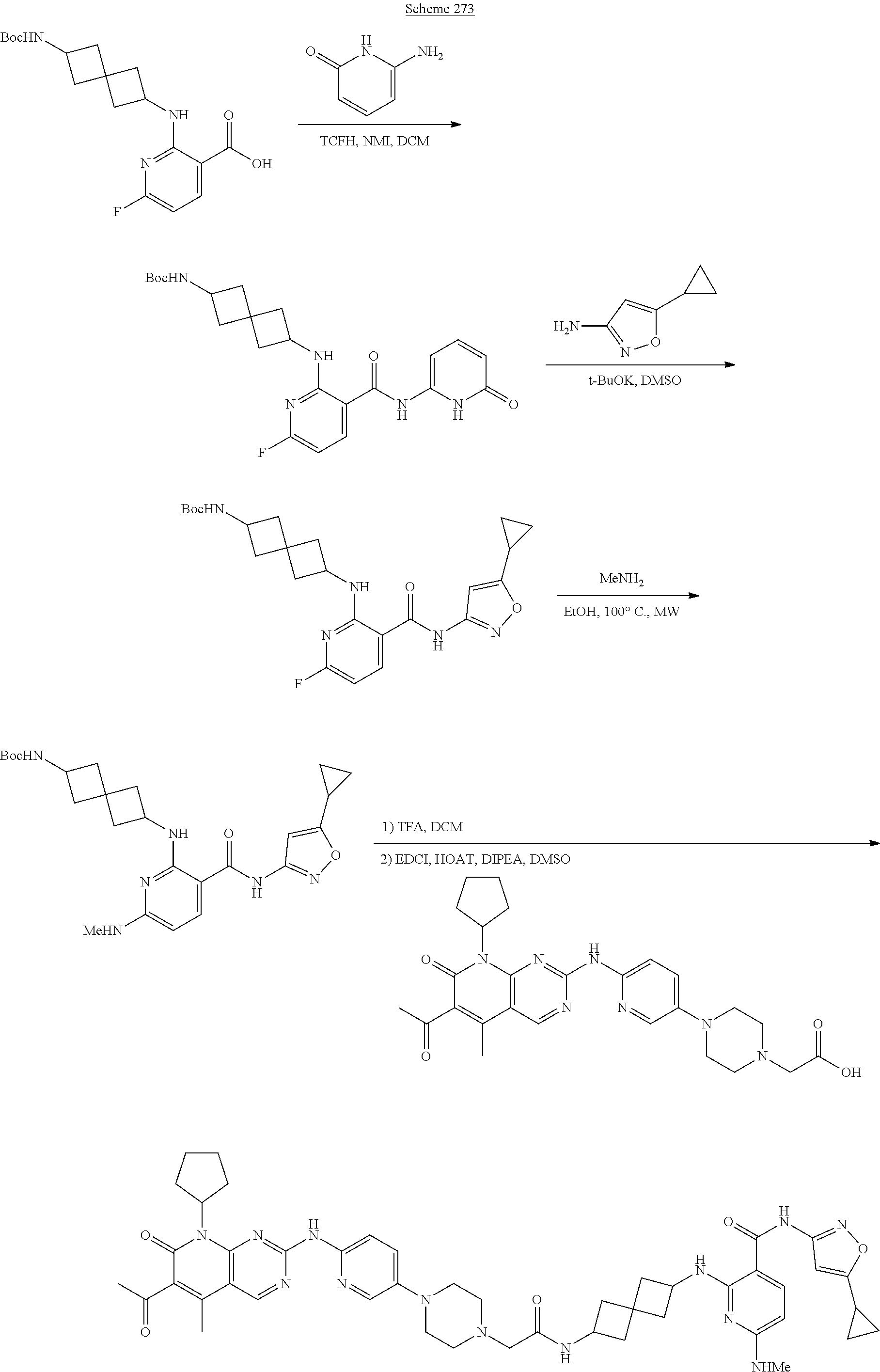

CPD-215 was synthesized following the standard procedures for preparing CPD-159 (7.6 mg, 11% yield over 5 steps) as a yellow solid. MS (ESI) m/z=870.7 [M+H]⁺.

### Example 274. 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-N-(6-(dimethylamino)pyridazin-3-yl)-6-(methylamino)nicotinamide (CPD-216)

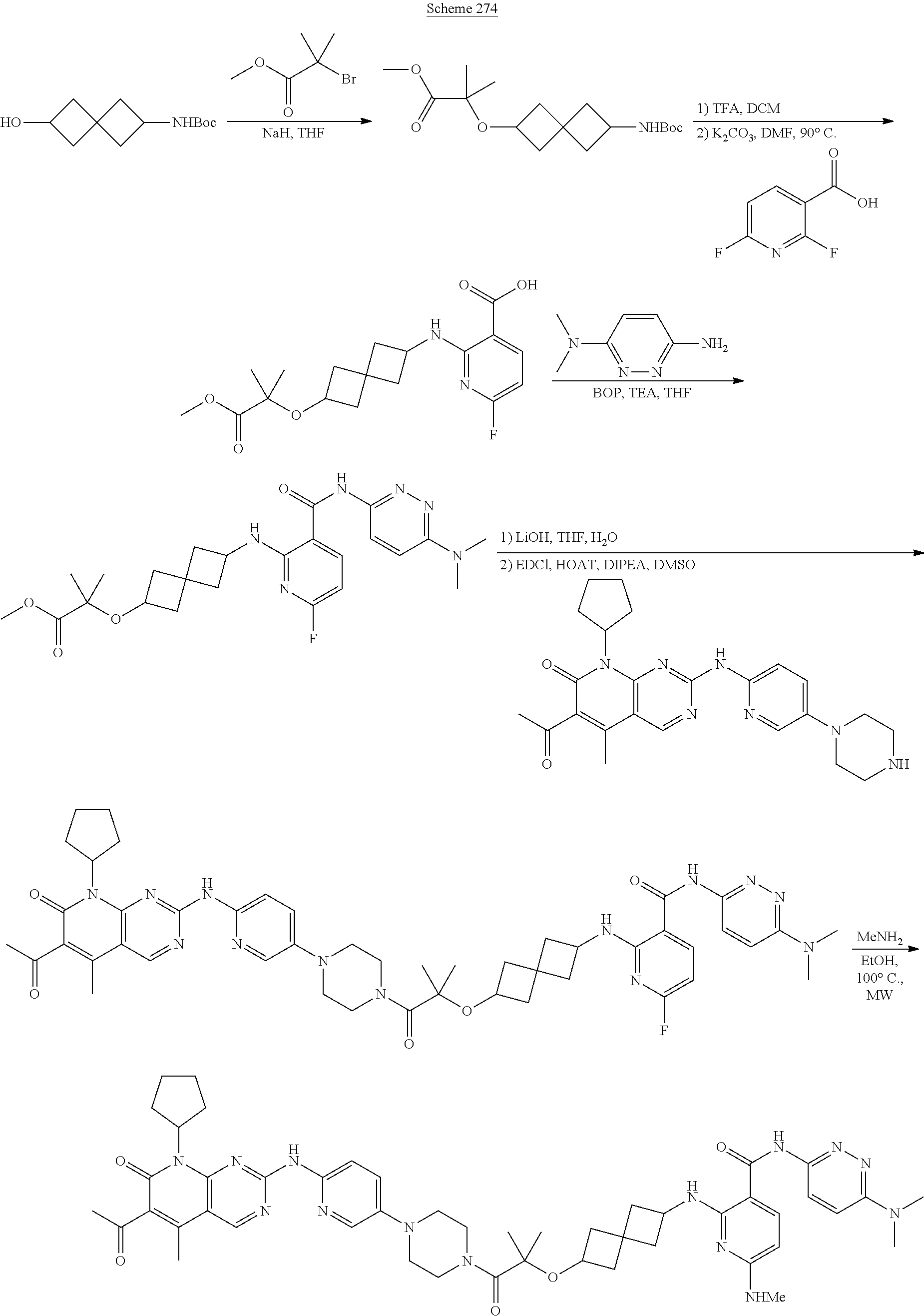

CPD-216 was synthesized following the standard procedures for preparing CPD-189 (5.1 mg, 2% yield over 7 steps) as a yellow solid. MS (ESI) m/z=913.8 [M+H]⁺.

### Example 275. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-(methylamino)nicotinamide (CPD-217)

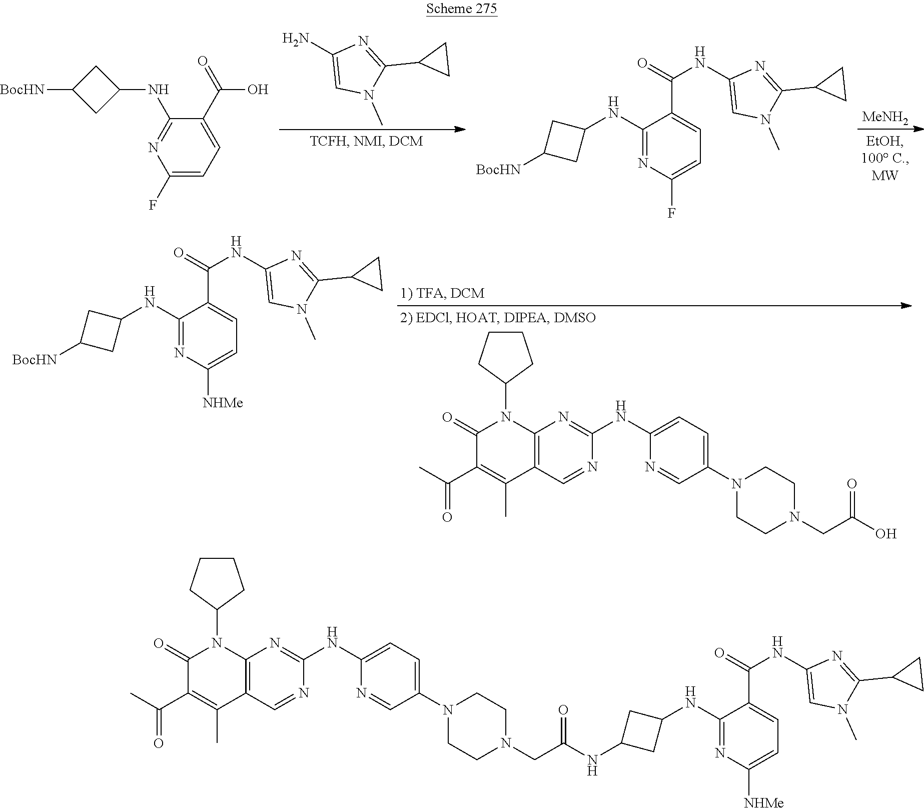

CPD-217 was synthesized following the standard procedures for preparing CPD-144 (3.0 mg, 3% yield over 4 steps) as a yellow solid. MS (ESI) m/z=843.8 [M+H]⁺.

### Example 276. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(6-(dimethylamino)pyridazin-3-yl)-6-(methylamino)nicotinamide (CPD-218)

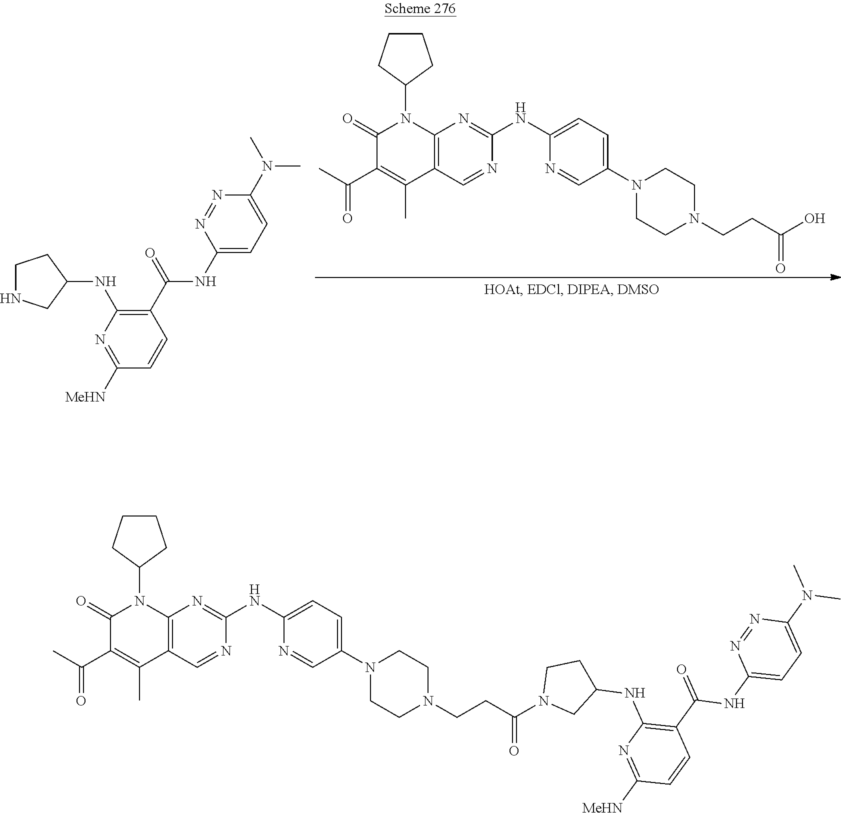

CPD-218 was synthesized following the standard procedure for preparing CPD-143 (7.0 mg, 10% yield) as a yellow solid. MS (ESI) n/z=858.7 [M+H]⁺.

### Example 277. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(6-(dimethylamino)pyridazin-3-yl)-6-(methylamino)nicotinamide (CPD-219)

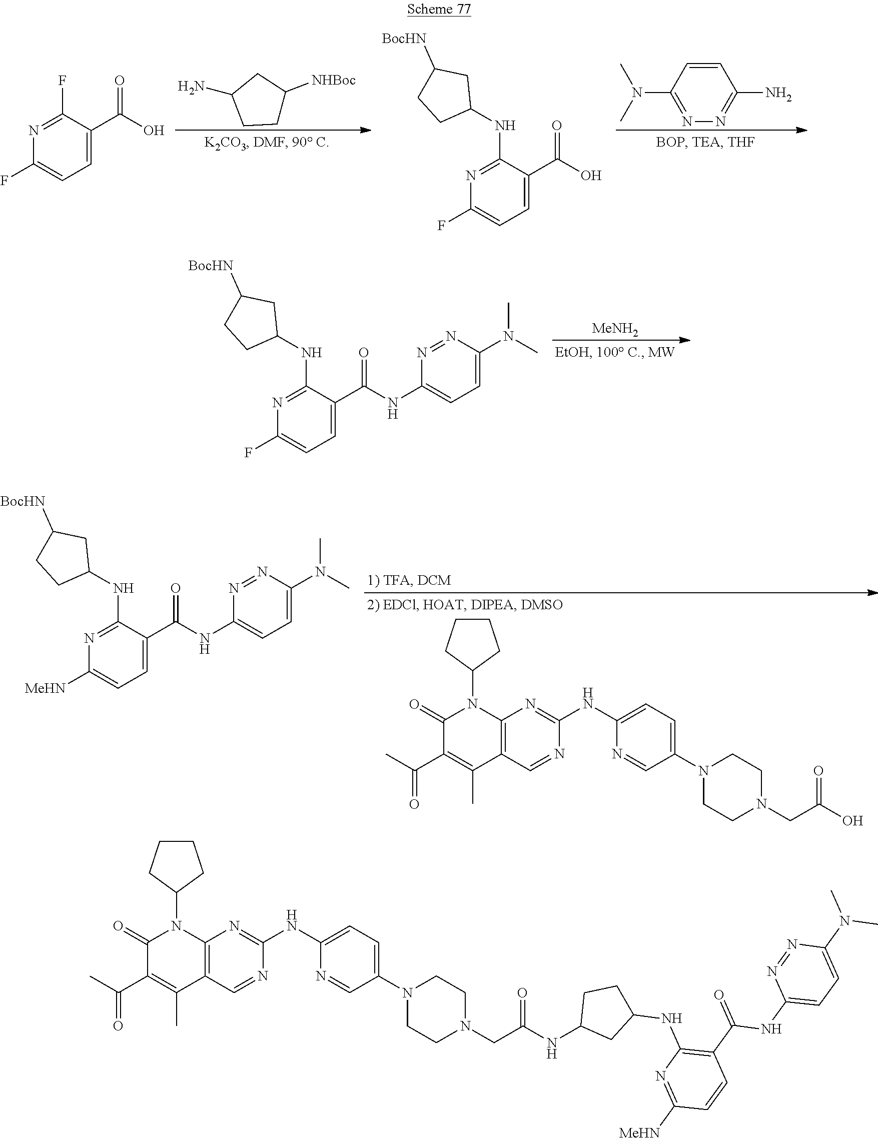

CPD-219 was synthesized following the standard procedures for preparing CPD-145 (7.6 mg, 6% yield over 5 steps) as a yellow solid. MS (ESI) m/z=858.7 [M+H]⁺.

### Example 278. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methoxy-6-methylpyridin-2-yl)-6-(methylamino)nicotinamide (CPD-220)

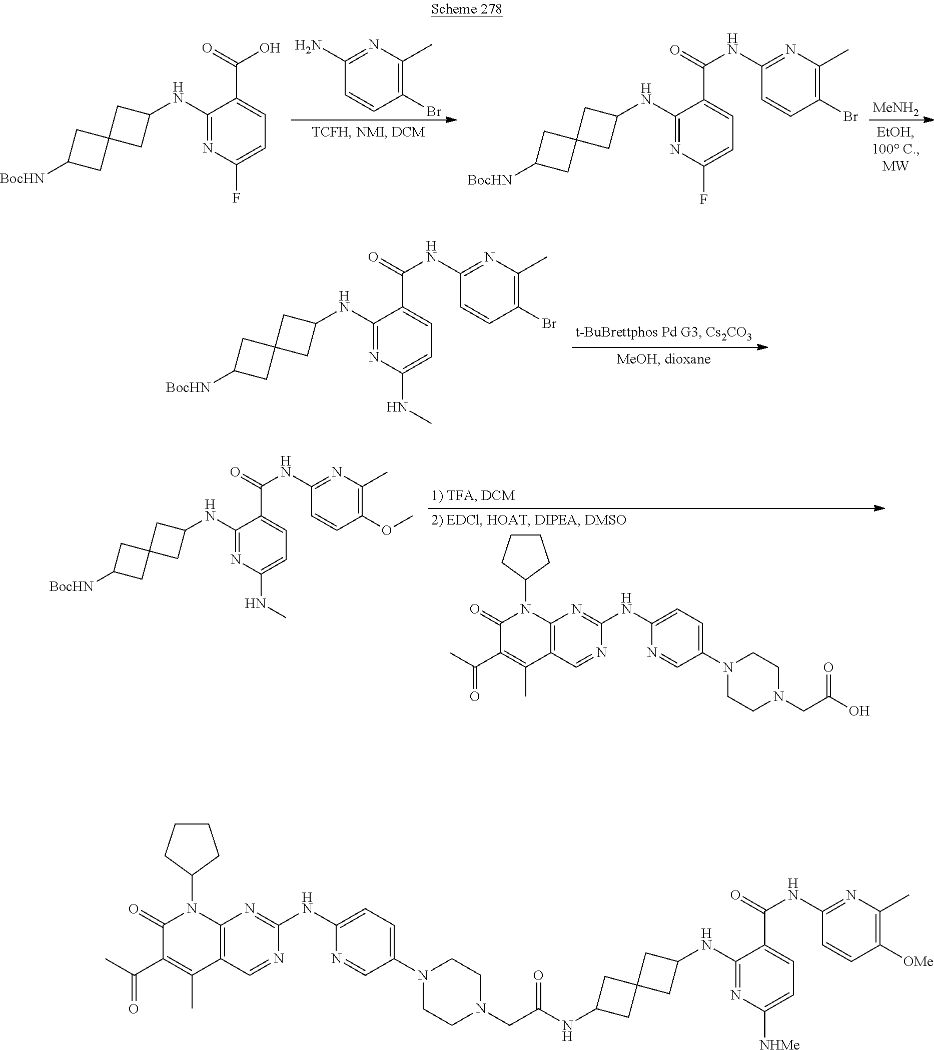

### Step 1. Synthesis of tert-butyl (6-((3-((5-bromo-6-methylpyridin-2-yl)carbamoyl)-6-fluoropyridin-2-yl)amino)spiro[3.3]heptan-2-yl)carbamate

To a solution of 2-((6-((tert-butoxycarbonyl)amino)spiro[3.3]heptan-2-yl)amino)-6-fluoronicotinic acid (60 mg, 164.20 μmol) in dichloromethane (5 mL) were added 5-bromo-6-methylpyridin-2-amine (30.71 mg, 164.20 μmol), TCFH (69.11 mg, 246.31 μmol) and NMI (47.18 mg, 574.72 μmol). The mixture was stirred at rt for 1 h. The reaction mixture was poured into water (50 mL) and extracted with ethyl acetate (3×20 mL). The combined organic layers were washed with saturated brine (30 mL), dried over anhydrous sodium sulfate, filtered, and evaporated under reduced pressure. The resulting residue was purified by reverse-phase chromatography to give the desired product (44 mg, 50% yield) as a pale-white solid. MS (ESI) m/z=534.4 [M+H]⁺.

### Step 2. Synthesis of tert-butyl (6-((3-((5-bromo-6-methylpyridin-2-yl)carbamoyl)-6-(methylamino)pyridin-2-yl)amino)spiro[3.3]heptan-2-yl)carbamate

A solution of tert-butyl (6-((3-((5-bromo-6-methylpyridin-2-yl)carbamoyl)-6-fluoropyridin-2-yl)amino)spiro[3.3]heptan-2-yl)carbamate (44 mg, 82.33 μmol) in MeNH₂ solution (30% in EtOH, 1 mL) was stirred at 100 [symbol] under microwave irradiation for 40 min. The reaction mixture was concentrated and purified by silica gel chromatography to give the desired product (39 mg, 87% yield) as a yellow solid. MS (ESI) m/z=545.4 [M+H]⁺.

### Step 3. Synthesis of tert-butyl (6-((3-((5-methoxy-6-methylpyridin-2-yl)carbamoyl)-6-(methylamino)pyridin-2-yl)amino)spiro[3.3]heptan-2-yl)carbamate

To a solution of tert-butyl (6-((3-((5-bromo-6-methylpyridin-2-yl)carbamoyl)-6-(methylamino)pyridin-2-yl)amino)spiro[3.3]heptan-2-yl)carbamate (39 mg, 71.50 mol) in dioxane (2 mL) and MeOH (2 mL) were added t-BuBrettphos Pd G3 (6.12 mg, 7.15 μmol) and Cs₂CO₃ (23.30 mg, 71.50 μmol) at rt. The mixture was stirred at 100 [symbol] under inert atmosphere for 16 h. The reaction mixture cooled to rt, poured into water (30 mL), and extracted with ethyl acetate (3×20 mL). The combined organic layers were washed with saturated brine (15 mL), dried over anhydrous sodium sulfate, filtered, and evaporated under reduced pressure. The resulting residue was purified by silica gel chromatography to give the desired product (31 mg, 87% yield) as a pale-white solid. MS (ESI) m/z=497.5 [M+H]⁺.

The remaining steps were performed following the standard procedures for preparing CPD-144 to provide the desired product (0.7 mg, 2% yield over 2 steps) as a yellow solid. MS (ESI) m/z=884.7 [M+H]⁺.

### Example 279. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-fluoro-6-methylpyridin-2-yl)-6-(methylamino)nicotinamide (CPD-221)

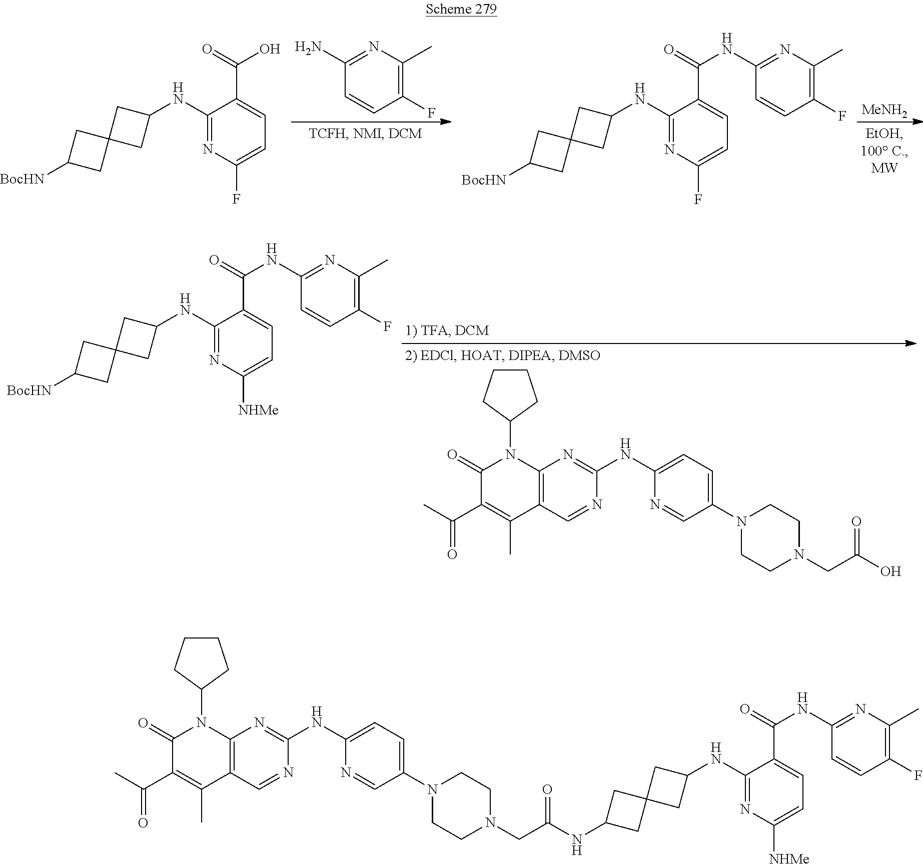

CPD-221 was synthesized following the standard procedures for preparing CPD-144 (31 mg, 19% yield over 4 steps) as a yellow solid. MS (ESI) m/z=872.7 [M+H]⁺.

### Example 280. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(isopropylamino)nicotinamide (CPD-222)

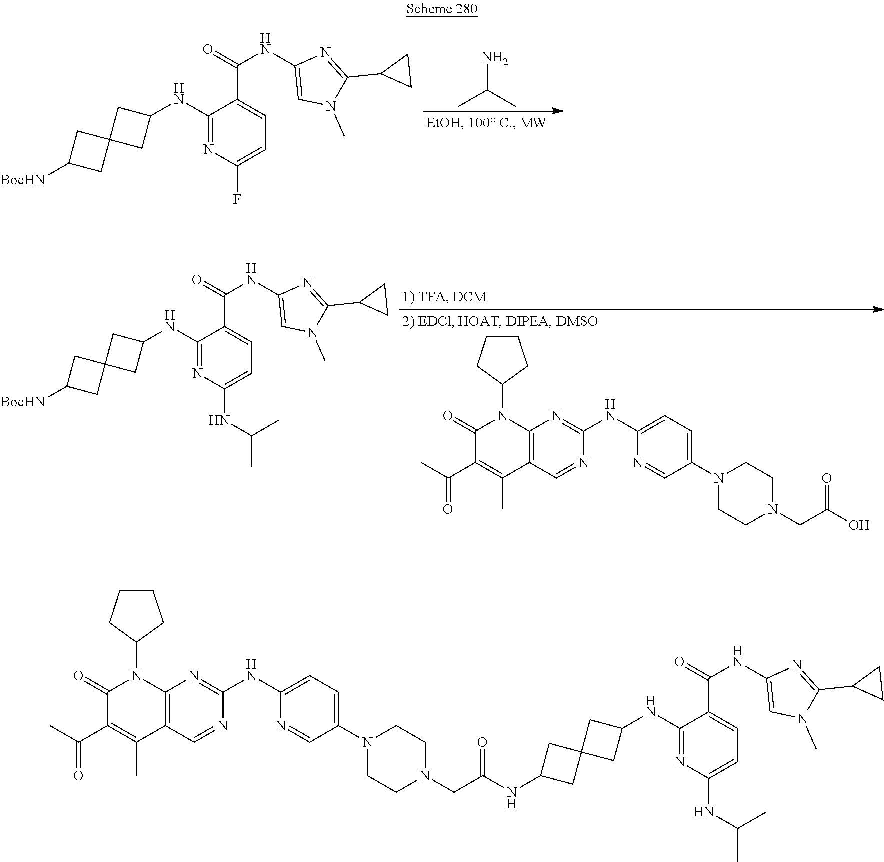

CPD-222 was synthesized following the standard procedures for preparing CPD-144 (12 mg, 33% yield over 3 steps) as a yellow solid. MS (ESI) m/z=911.7 [M+H]⁺.

### Example 281. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclohexyl)amino)-N-(6-(dimethylamino)pyridazin-3-yl)-6-(methylamino)nicotinamide (CPD-223)

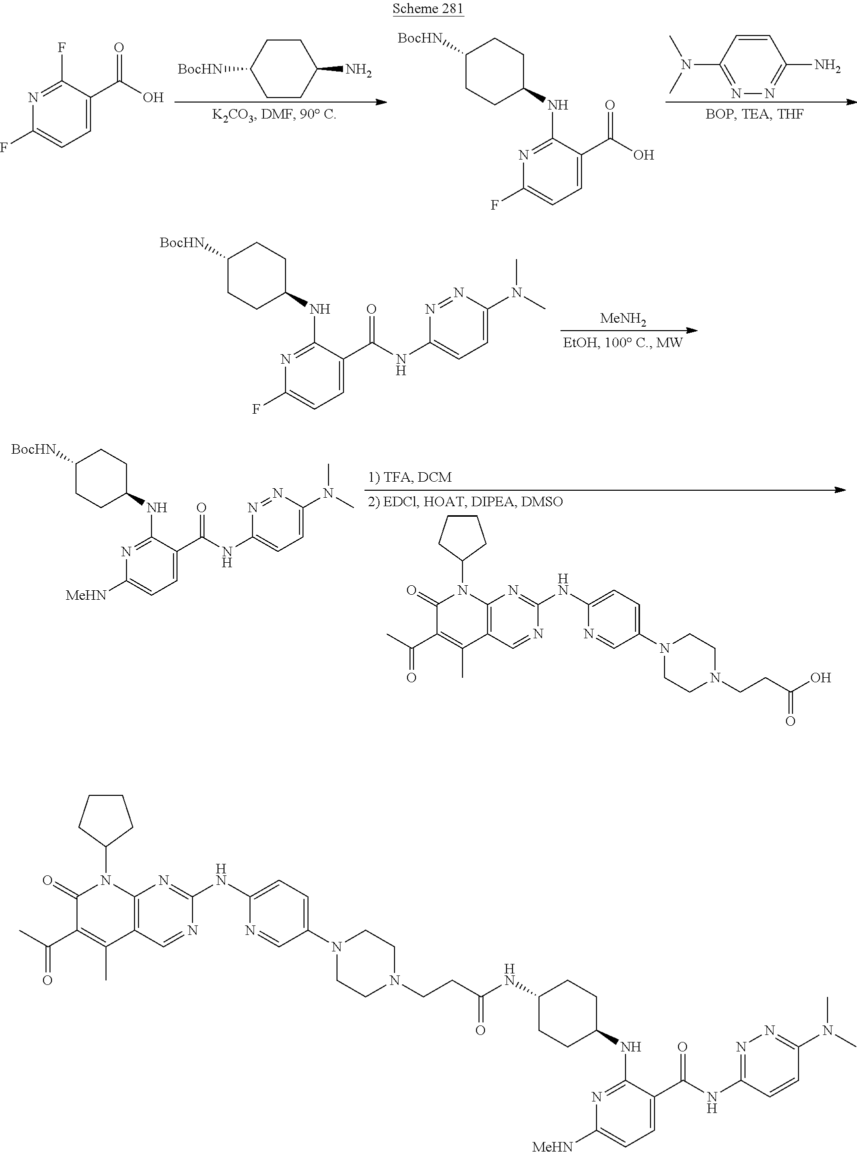

CPD-223 was synthesized following the standard procedures for preparing CPD-145 (6.9 mg, 1% yield over 5 steps) as a yellow solid. MS (ESI) m/z=886.6 [M+H]⁺.

### Example 282. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-(methylamino)nicotinamide (CPD-224)

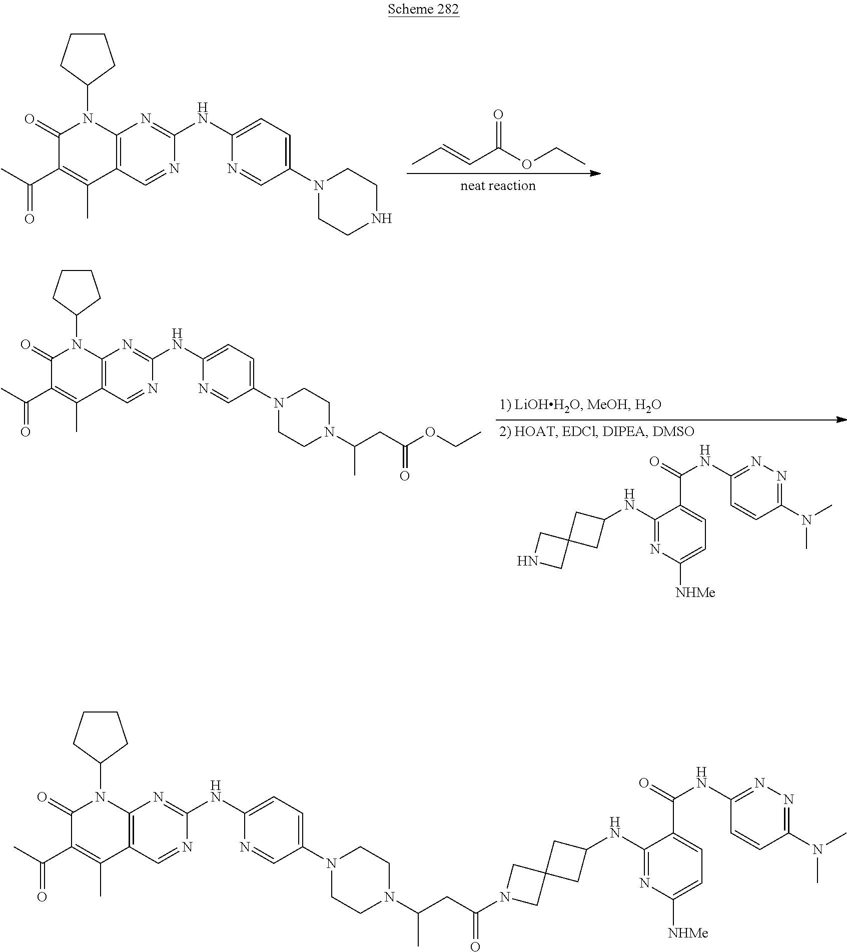

### Step 1. Synthesis of ethyl 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoate

A solution of 6-acetyl-8-cyclopentyl-5-methyl-2-[(5-piperazin-1-yl-2-pyridyl)amino]pyrido[2,3-d]pyrimidin-7-one (50 mg, 0.11 mmol) in ethyl (E)-but-2-enoate (1 ml) was stirred at 90° C. for 12 h. After cooling down to rt, the solution was diluted with water (20 mL) and extracted with ethyl acetate (2×30 mL). The combined organic layers were washed with saturated brine (20 mL), dried over anhydrous sodium sulfate, filtered, and evaporated under reduced pressure. The resulting residue was purified by silica gel chromatography to provide the desired product (10 mg, 16% yield) as a light-yellow solid. MS (ESI) m/z=562.5 [M+H]⁺.

The remaining steps were performed following the standard procedures for preparing CPD-82 to provide the desired product (2 mg, 12% yield over 2 steps) as a yellow solid. MS (ESI) m/z=898.7 [M+H]⁺.

### Example 283. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-((methyl-d₃)amino)-N-(5-methylisoxazol-3-yl)nicotinamide (CPD-225)

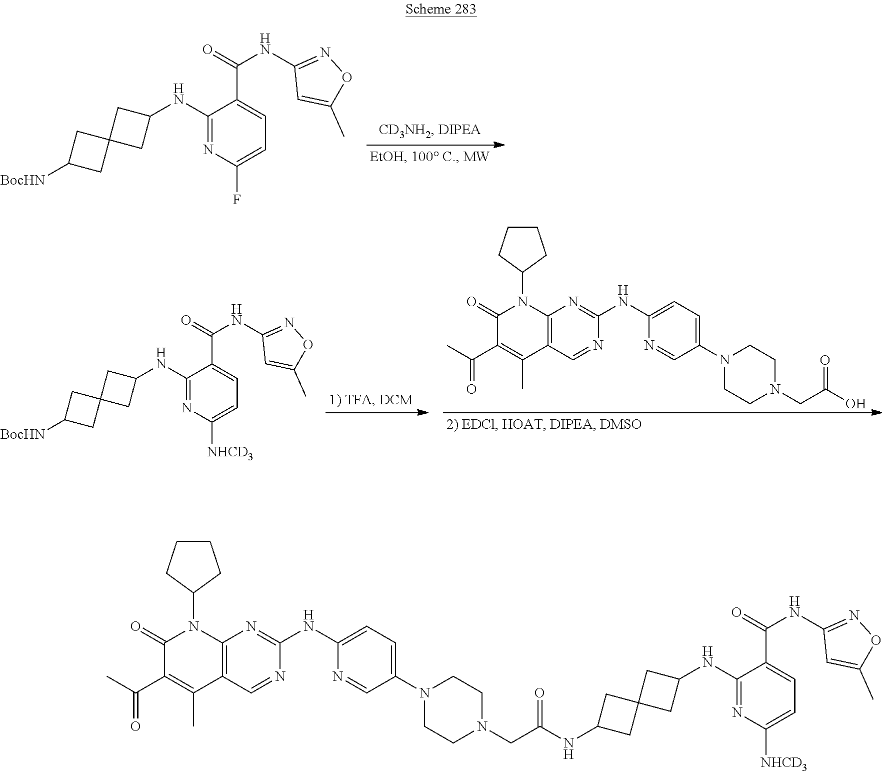

CPD-225 was synthesized following the standard procedures for preparing CPD-144 (6.0 mg, 10% yield over 3 steps) as a yellow solid. MS (ESI) m/z=847.8 [M+H]⁺.

### Example 284. 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-6-((methyl-d₃)amino)-N-(6-methylpyridin-2-yl)nicotinamide (CPD-226)

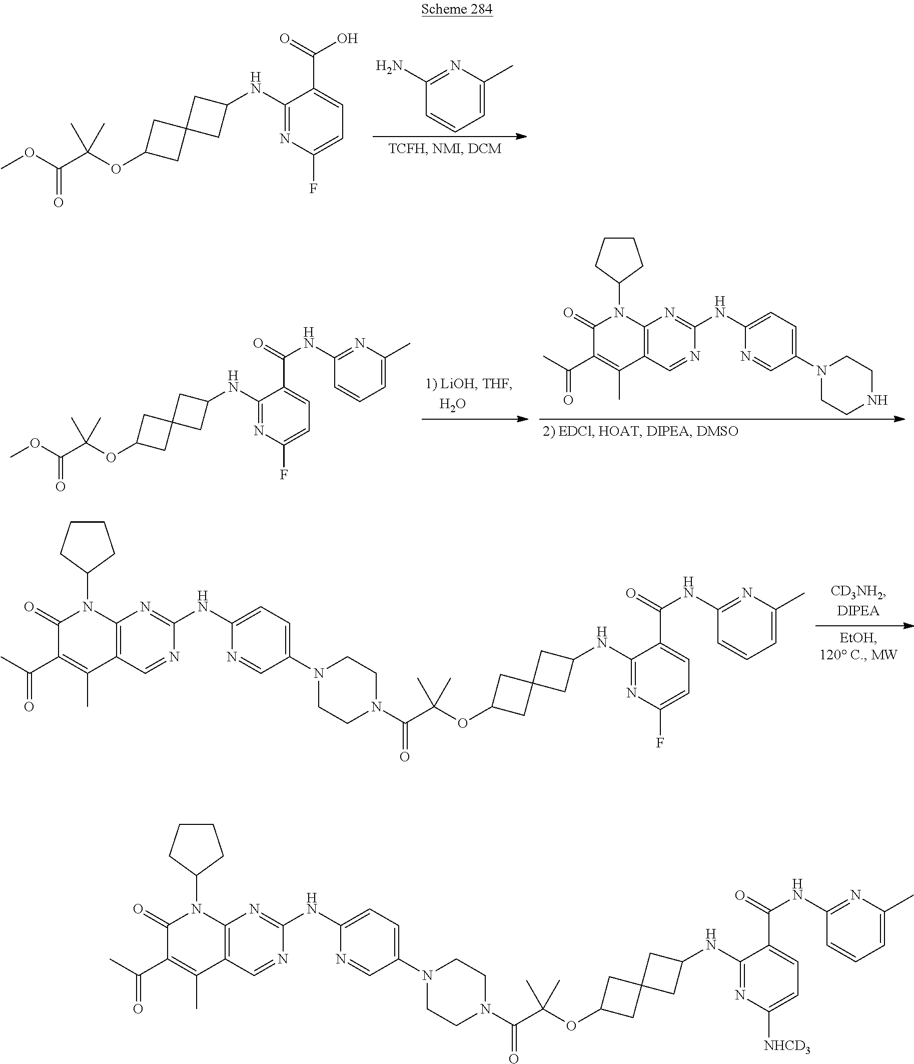

### Step 1. Synthesis of methyl 2-((6-((6-fluoro-3-((6-methylpyridin-2-yl)carbamoyl)pyridin-2-yl)amino)spiro[3.3]heptan-2-yl)oxy)-2-methylpropanoate

A mixture of 6-fluoro-2-((6-((1-methoxy-2-methyl-1-oxopropan-2-yl)oxy)spiro[3.3]heptan-2-yl)amino)nicotinic acid (12 mg, 0.03 mmol), 6-methylpyridin-2-amine (7 mg, 0.06 mmol), TCFH (18 mg, 0.06 mmol) and NMI (13 mg, 0.16 mmol) in dichloromethane (2 mL) was stirred at rt for 2 h. The reaction mixture was poured into water (20 mL) and extracted with ethyl acetate (3×20 mL). The combined organic layers were washed with saturated brine (50 mL), dried over anhydrous Na₂SO₄, filtered, and evaporated under reduced pressure. The resulting residue was purified by silica gel flash chromatography to give the desired product (11 mg, 73% yield) as a colorless oil. MS (ESI) m/z=457.5[M+H]⁺.

### Step 2. Synthesis of 2-((6-((6-fluoro-3-((6-methylpyridin-2-yl)carbamoyl)pyridin-2-yl)amino)spiro[3.3]heptan-2-yl)oxy)-2-methylpropanoic acid

A mixture of methyl 2-((6-((6-fluoro-3-((6-methylpyridin-2-yl)carbamoyl)pyridin-2-yl)amino)spiro[3.3]heptan-2-yl)oxy)-2-methylpropanoate (11 mg, 0.02 mmol) and LiOH (6 mg, 0.2 mmol) in MeOH (1 mL) and H₂O (1 mL) was stirred at rt for 2 h. The resulting mixture was concentrated and purified by reverse-phase chromatography to give the desired product (9.1 mg, 67% yield) as a white solid. MS (ESI) m/z=443.6 [M+H]⁺.

### Step 3. Synthesis of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-6-fluoro-N-(6-methylpyridin-2-yl)nicotinamide

A mixture of 2-((6-((6-fluoro-3-((6-methylpyridin-2-yl)carbamoyl)pyridin-2-yl)amino)spiro[3.3]heptan-2-yl)oxy)-2-methylpropanoic acid (8.5 mg, 0.02 mmol), 6-acetyl-8-cyclopentyl-5-methyl-2-((5-(piperazin-1-yl)pyridin-2-yl)amino)pyrido[2,3-d]pyrimidin-7(8H)-one (6.8 mg, 0.02 mmol), EDCI (4.5 mg, 0.03 mmol), HOAt (3.5 mg, 0.03 mmol) and DIPEA (10 mg, 0.07 mmol) in DMSO (2 mL) was stirred at rt for 16 h. The reaction mixture was purified by reverse-phase chromatography to give the desired product (7.3 mg, 54% yield) as a yellow solid. MS (ESI) m/z=872.7 [M+H]⁺.

### Step 4. Synthesis of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-6-((methyl-d3)amino)-N-(6-methylpyridin-2-yl)nicotinamide

To a mixture of 2-((6-((1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methyl-1-oxopropan-2-yl)oxy)spiro[3.3]heptan-2-yl)amino)-6-fluoro-N-(6-methylpyridin-2-yl)nicotinamide (7.3 mg, 0.008 mmol) in EtOH (1 mL) were added methan-d₃-amine (1.4 mg, 0.04 mmol) and DIPEA (21 mg, 0.16 mmol). The reaction mixture was stirred at 120° C. for 2 h under microwave irradiation. The mixture was concentrated and purified by pre-TLC to give the desired product (1.9 mg, 22% yield) as a white solid. MS (ESI) m/z=886.8 [M+H]⁺.

### Example 285. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-isopropylisoxazol-3-yl)-6-(methylamino)nicotinamide (CPD-227)

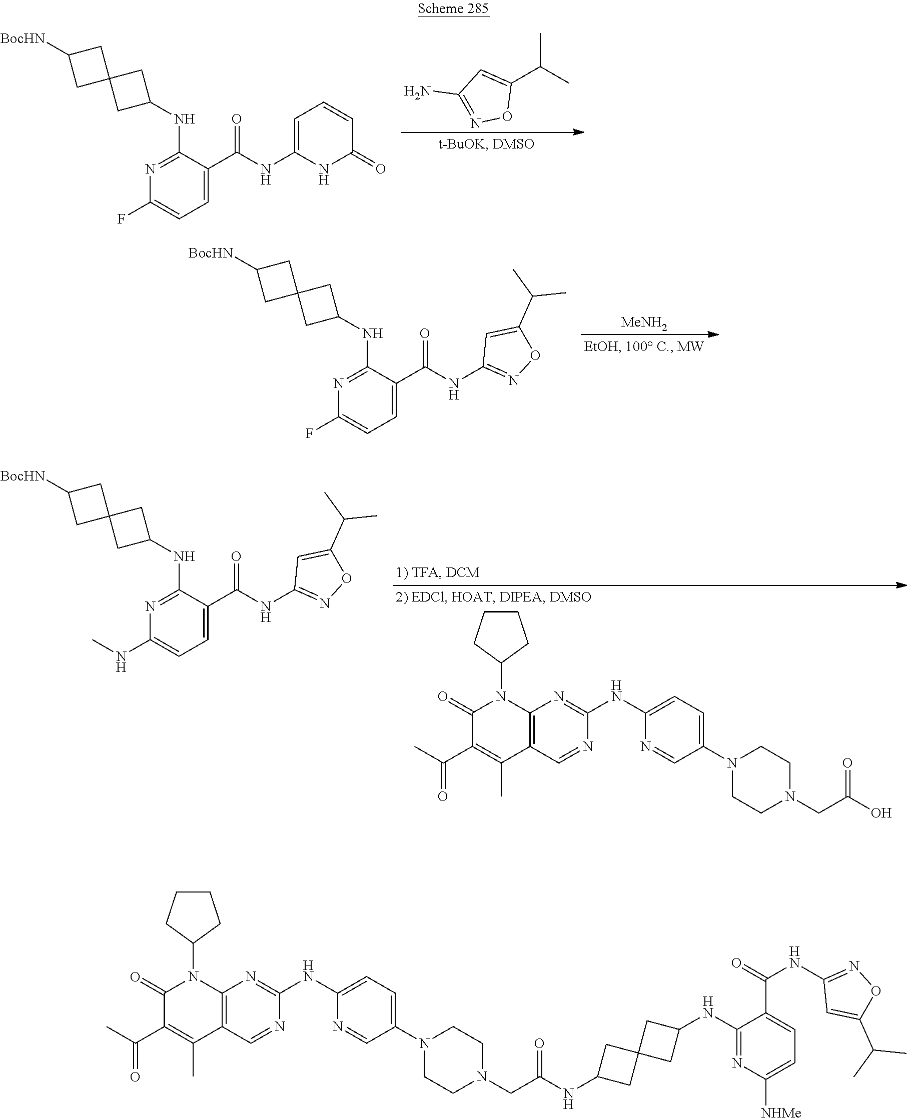

CPD-227 was synthesized following the standard procedures for preparing CPD-159 (10.7 mg, 10% yield over 4 steps) as a yellow solid. MS (ESI) m/z=872.7 [M+H]⁺.

### Example 286. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(1,5-dimethyl-1H-pyrazol-3-yl)-6-(methylamino)nicotinamide (CPD-228)

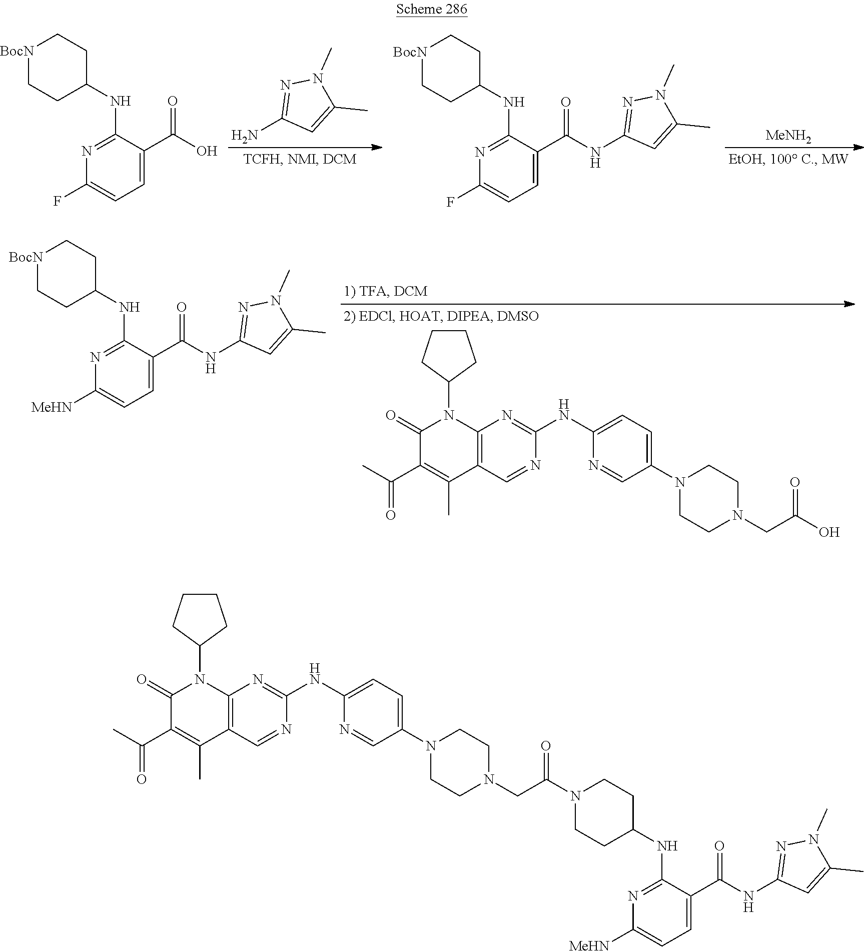

CPD-228 was synthesized following the standard procedures for preparing CPD-144 (22.0 mg, 28% yield over 4 steps) as a yellow solid. MS (ESI) m/z=831.7 [M+H]⁺.

### Example 287. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-(methylamino)-N-(5-methylisoxazol-3-yl)nicotinamide (CPD-229)

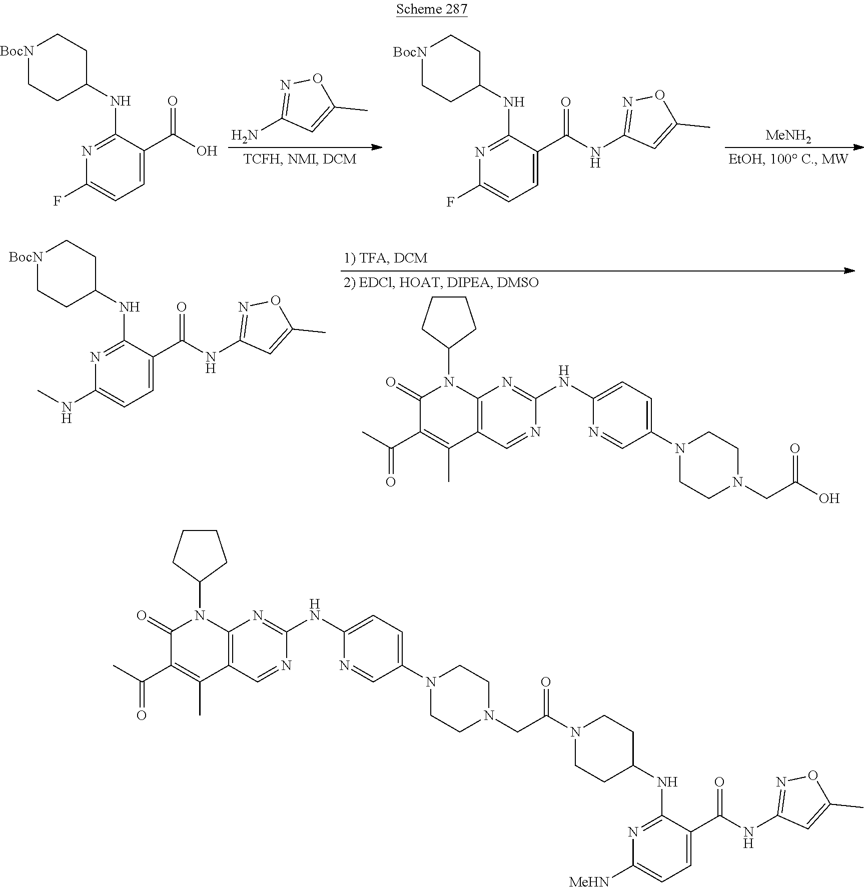

CPD-229 was synthesized following the standard procedures for preparing CPD-144 (6.5 mg, 6% yield over 4 steps) as a yellow solid. MS (ESI) m/z=818.6 [M+H]⁺.

### Example 288. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(1,5-dimethyl-1H-pyrazol-3-yl)-6-((methyl-d₃)amino)nicotinamide (CPD-230)

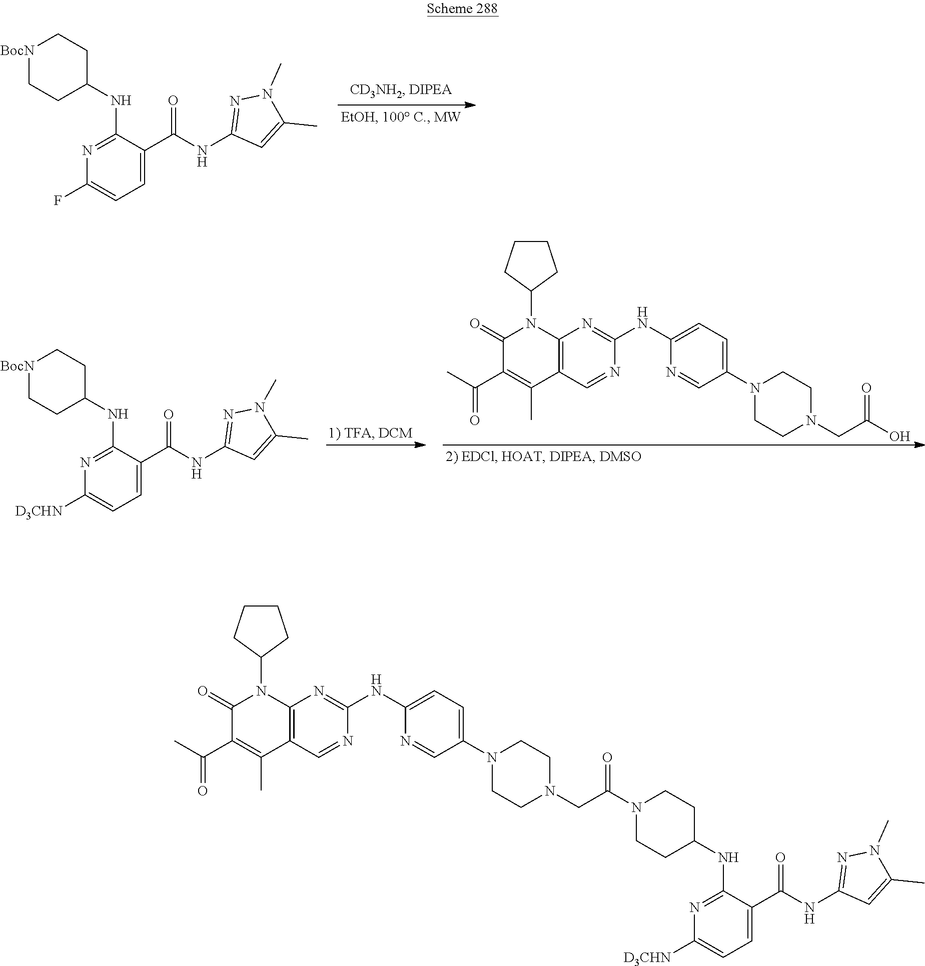

CPD-230 was synthesized following the standard procedures for preparing CPD-144 (6.1 mg, 13% yield over 3 steps) as a yellow solid. MS (ESI) m/z=834.6 [M+H]⁺.

### Example 289. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-((methyl-d₃)amino)-N-(5-methylisoxazol-3-yl)nicotinamide (CPD-231)

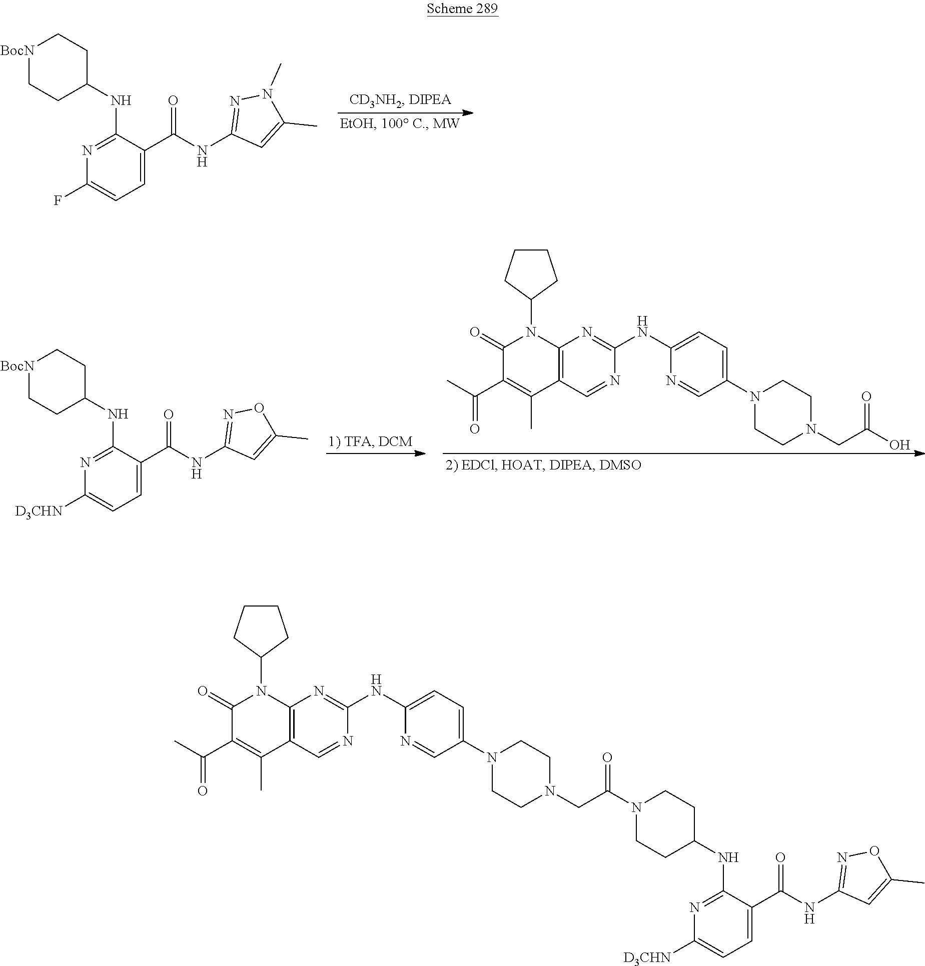

CPD-231 was synthesized following the standard procedures for preparing CPD-144 (6.2 mg, 33% yield over 3 steps) as a yellow solid. MS (ESI) m/z=821.5 [M+H]⁺.

### Example 290. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-((methyl-d₃)amino)-N-(6-methylpyridin-2-yl)nicotinamide (CPD-232)

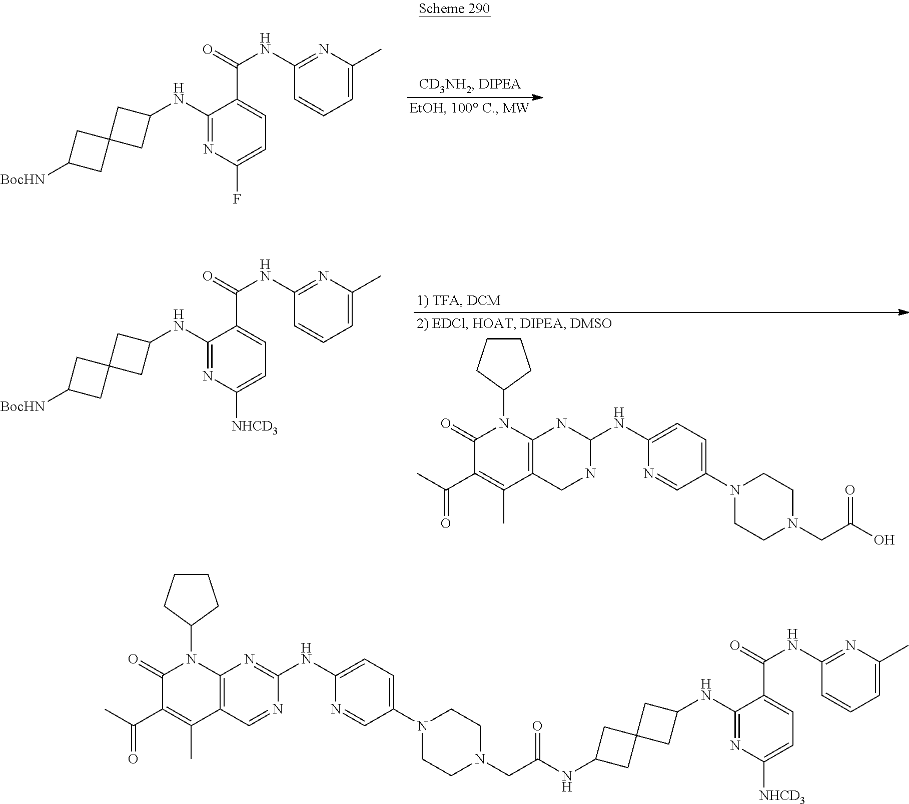

CPD-232 was synthesized following the standard procedures for preparing CPD-144 (5.0 mg, 9% yield over 3 steps) as a yellow solid. MS (ESI) m/z=857.6 [M+H]⁺.

### Example 291. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-N-(1,5-dimethyl-1H-pyrazol-3-yl)-6-((methyl-d₃)amino)nicotinamide (CPD-233)

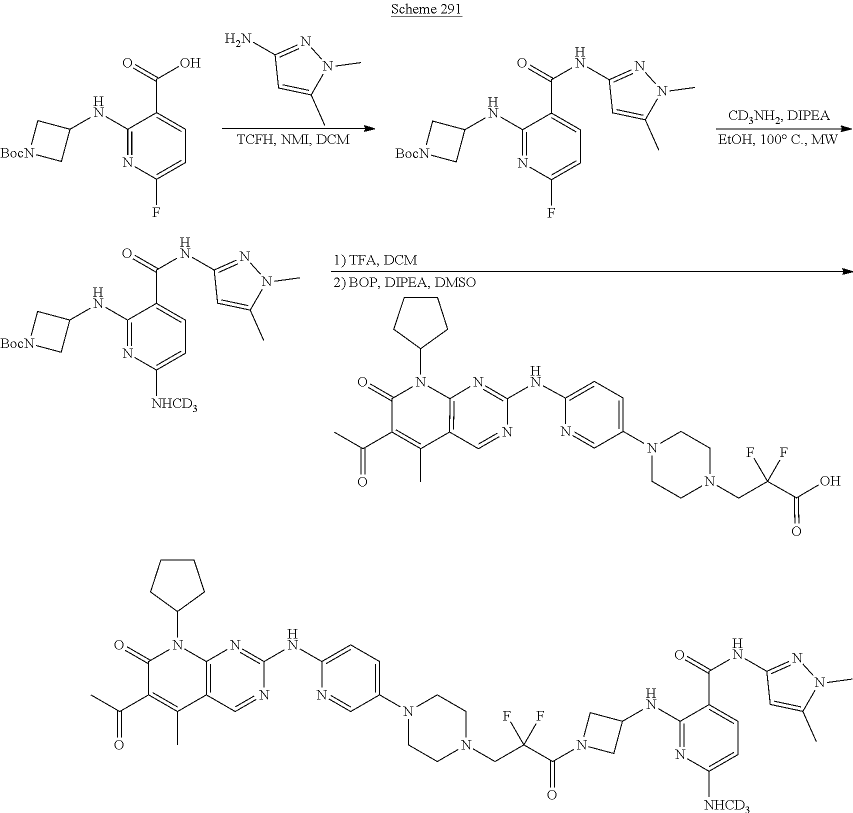

CPD-233 was synthesized following the standard procedures for preparing CPD-144 (7.0 mg, 15% yield over 4 steps) as a yellow solid. MS (ESI) m/z=856.6 [M+H]⁺.

### Example 292. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-6-((methyl-d3)amino)-N-(5-methylisoxazol-3methyl-yl)nicotinamide (CPD-234)

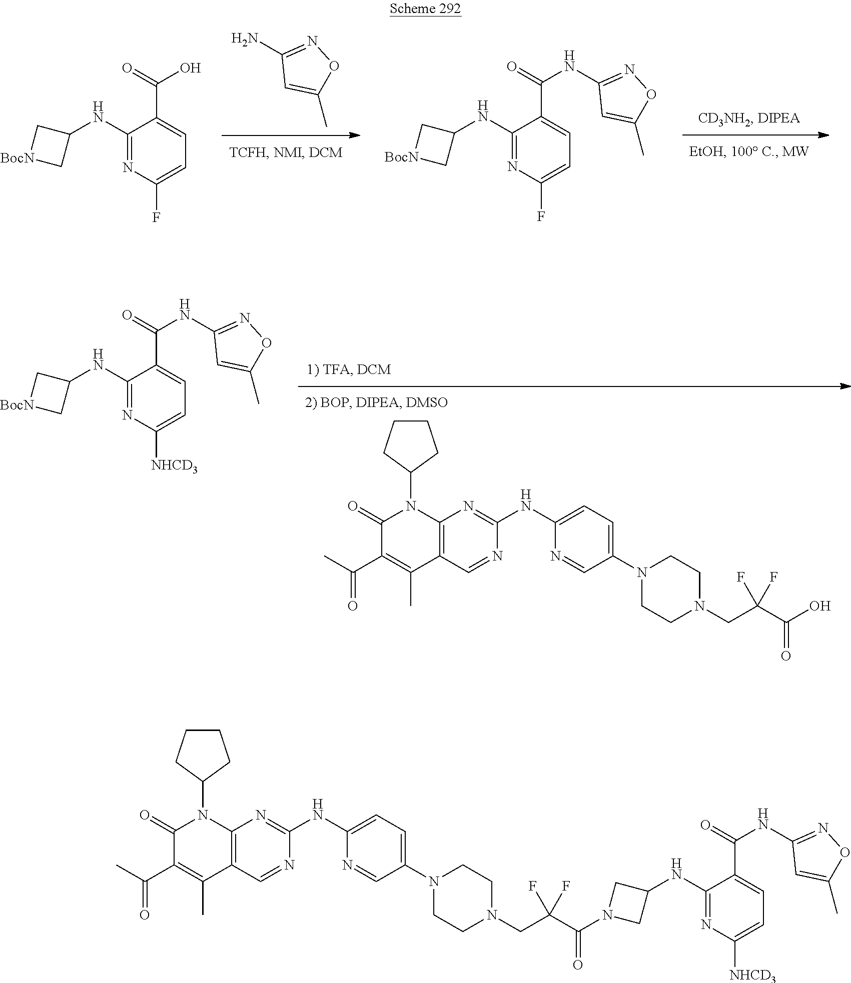

CPD-234 was synthesized following the standard procedures for preparing CPD-144 (8.0 mg, 10% yield over 4 steps) as a yellow solid. MS (ESI) m/z=843.6 [M+H]⁺.

### Example 293. 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-2-azaspiro[3.4]octan-6-yl)amino)-N-(6-(dimethylamino)pyridazin-3-yl)-6-(methylamino)nicotinamide (CPD-235)

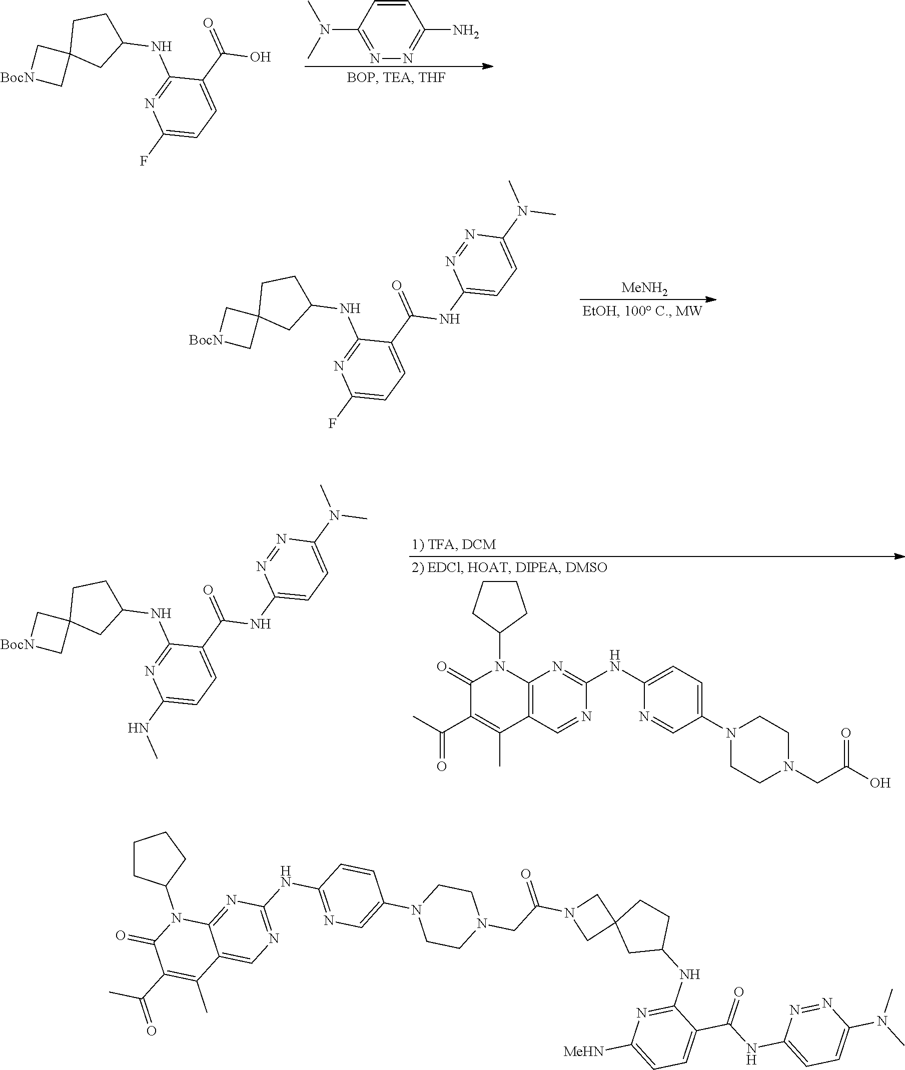

CPD-235 was synthesized following the standard procedures for preparing CPD-145 (2.0 mg, 2% yield over 4 steps) as a yellow solid. MS (ESI) m/z=884.5 [M+H]⁺.

### Example 294. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(2-cyclopropyl-1-methyl-1H-imidazol-4-yl)-6-((methyl-d₃)amino)nicotinamide (CPD-236)

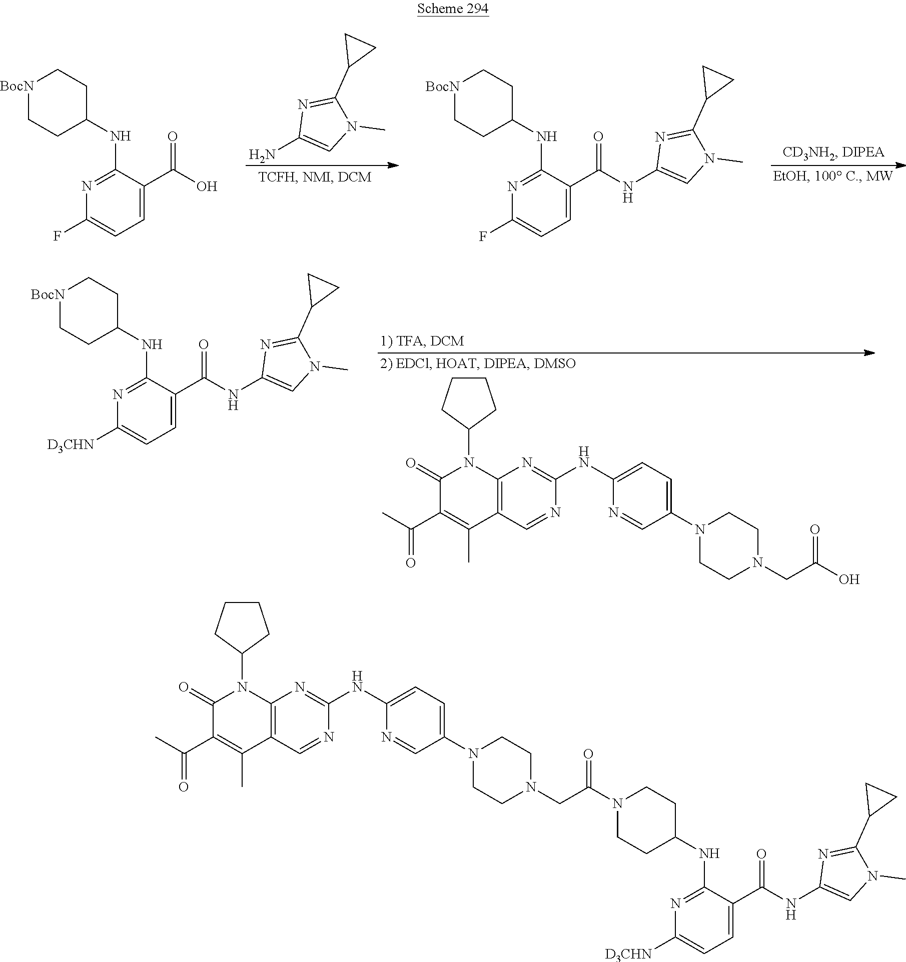

CPD-236 was synthesized following the standard procedures for preparing CPD-144 (5.7 mg, 4% yield over 4 steps) as a yellow solid. MS (ESI) m/z=860.7 [M+H]⁺.

### Example 295. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-((methyl-d3)amino)nicotinamide (CPD-237)

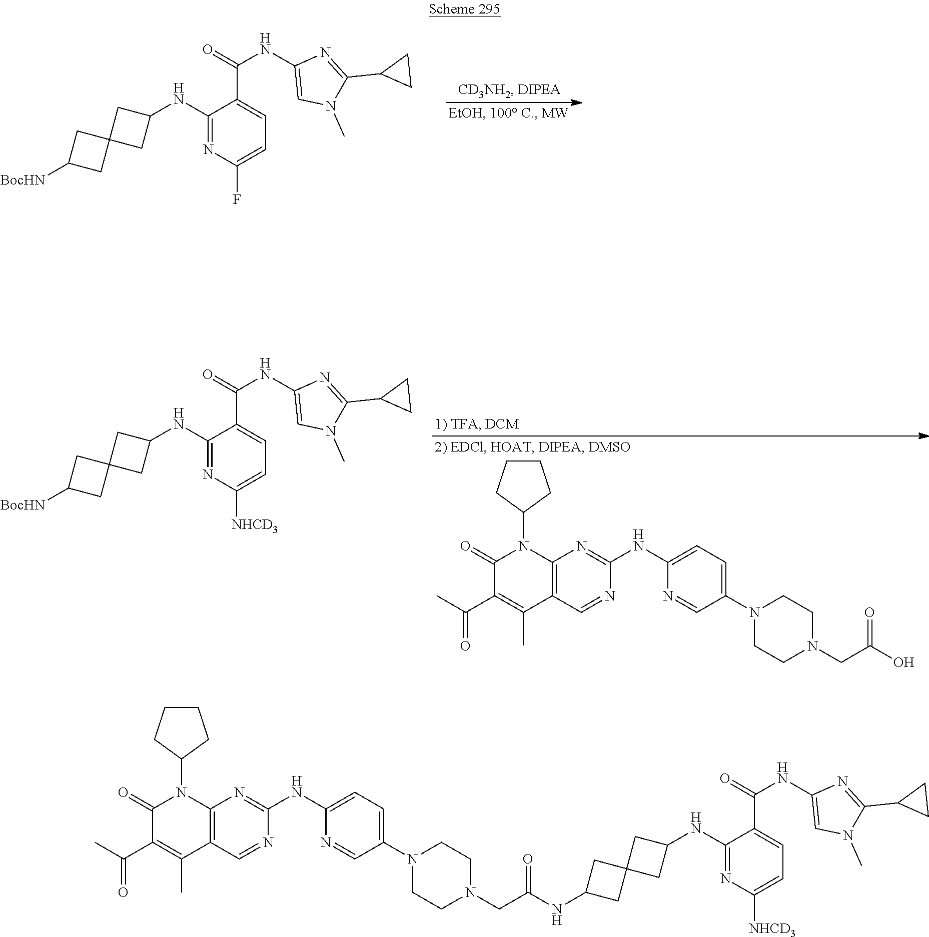

CPD-237 was synthesized following the standard procedures for preparing CPD-144 (5.5 mg, 20% yield over 3 steps) as a yellow solid. MS (ESI) m/z=886.7 [M+H]⁺.

### Example 296. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-isopropyl-2-methyl-1H-imidazol-4-yl)-6-(methylamino)nicotinamide (CPD-238)

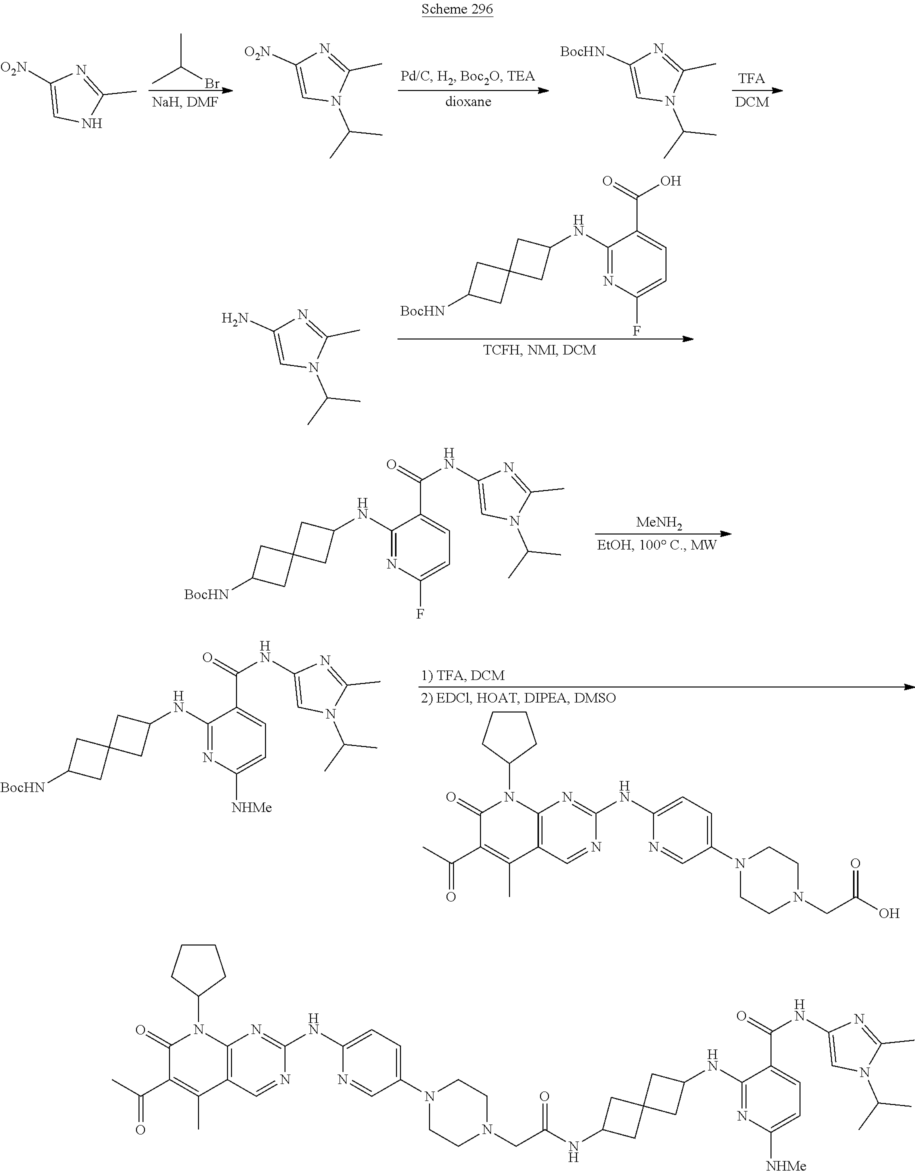

### Step 1. Synthesis of 1-isopropyl-2-methyl-4-nitro-1H-imidazole

To a solution of 2-methyl-4-nitro-1H-imidazole (500 mg, 3.93 mmol) in DMF (10 mL) was added NaH (236.01 mg, 5.90 mmol, 60 wt % in mineral oil) at 0 [symbol] . After stirring at the same temperature for 0.5 h, 2-bromopropane (2.42 g, 19.67 mmol) was added to the mixture. The mixture was stirred at 100 [symbol] for 16 h. The reaction mixture was poured into water (100 mL), acidified with aq. HCl (1 M), and extracted with ethyl acetate (3×50 mL). The combined organic layers were washed with saturated brine (75 mL), dried over anhydrous sodium sulfate, filtered, and evaporated under reduced pressure. The resulting residue was purified by silica gel chromatography to give the desired product (570 mg, 86% yield) as a pale-white solid. MS (ESI) m/z=170.2 [M+H]⁺.

### Step 2. Synthesis of tert-butyl (1-isopropyl-2-methyl-1H-imidazol-4-yl)carbamate

To a solution of 1-isopropyl-2-methyl-4-nitro-1H-imidazole (200 mg, 1.18 mmol) in dioxane (5 mL) were added 10% Pd/C (20 mg), Boc₂O (386.57 mg, 1.77 mmol) and TEA (239.25 mg, 2.36 mmol). The mixture was stirred at rt under hydrogen atmosphere for 2 h. The reaction mixture was filtered and concentrated to give the crude. The resulting residue was purified by silica gel chromatography to give the desired product (180 mg, 64% yield) as a pale-white solid. MS (ESI) m/z=240.4 [M+H]⁺.

### Step 3. Synthesis of 1-isopropyl-2-methyl-1H-imidazol-4-amine

A solution of tert-butyl (1-isopropyl-2-methyl-1H-imidazol-4-yl)carbamate (70 mg, 292.50 μmol) in dichloromethane (2 mL) and TFA (1 mL) was stirred at rt for 1 h. The reaction mixture was filtered and evaporated under reduced pressure to give the crude product (40 mg, 98% yield) as a yellow oil, which was used directly to the next step without further purification. MS (ESI) m/z=140.2 [M+H]⁺.

The remaining steps were performed following the standard procedures for preparing CPD-144 to provide the desired product (8.5 mg, 14% yield over 4 steps) as a yellow solid. MS (ESI) m/z=885.5 [M+H]⁺.

### Example 297. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-cyclopropyl-5-fluoropyridin-2-yl)-6-((methyl-da)amino)nicotinamide (CPD-239)

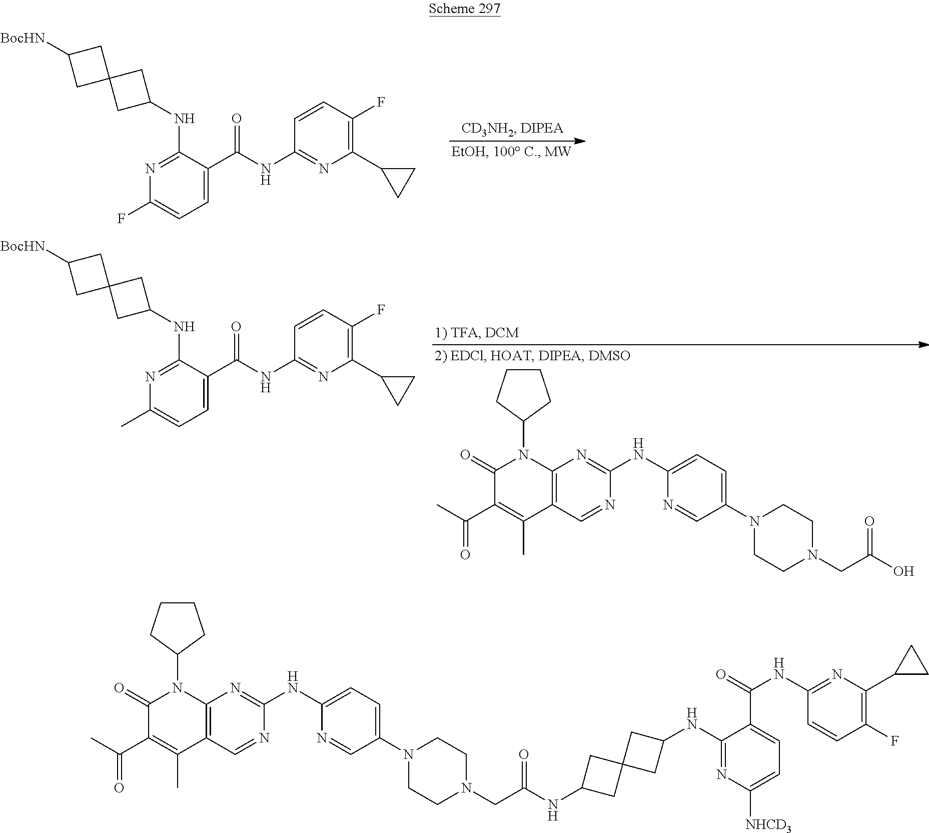

CPD-239 was synthesized following the standard procedures for preparing CPD-144 (0.83 mg, 5% yield over 3 steps) as a yellow solid. MS (ESI) m/z=901.7 [M+H]⁺.

### Example 298. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-cyclopropylpyridin-2-yl)-6-(methylamino)nicotinamide (CPD-240)

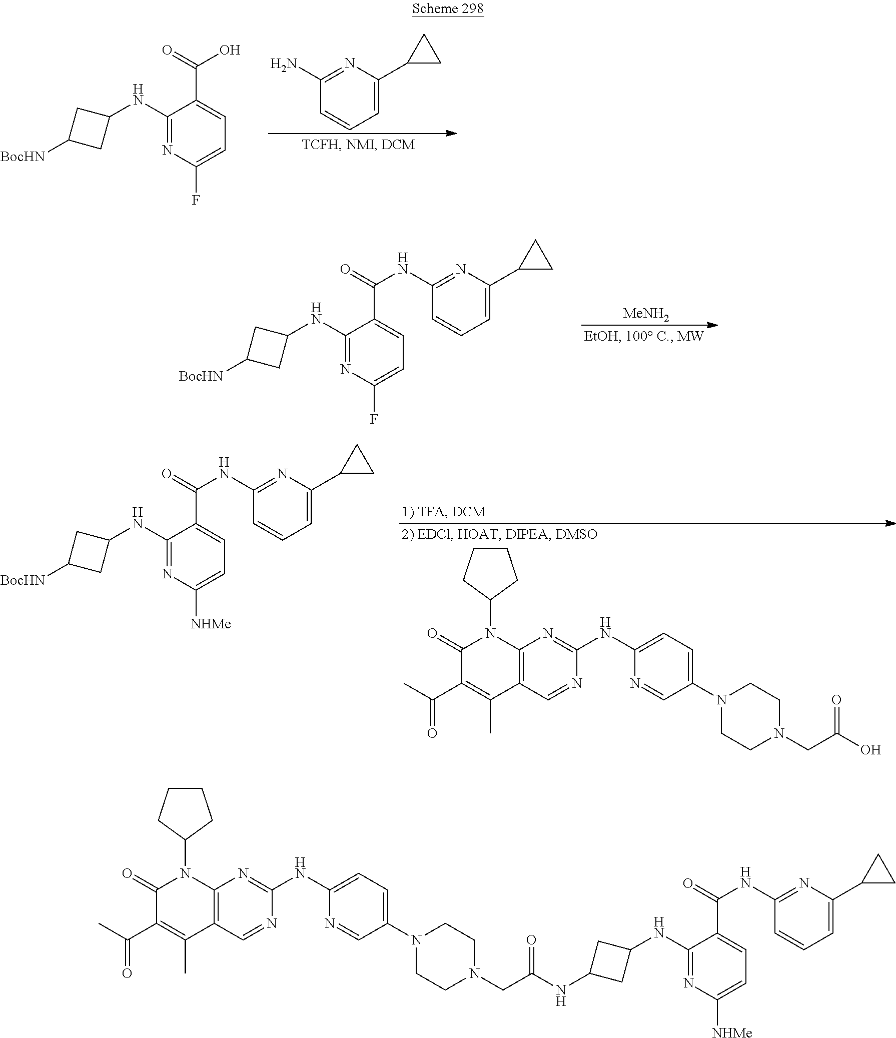

CPD-240 was synthesized following the standard procedures for preparing CPD-144 (2.6 mg, 3% yield over 4 steps) as a yellow solid. MS (ESI) m/z=840.6 [M+H]⁺.

### Example 299. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-((methyl-d3)amino)nicotinamide (CPD-241)

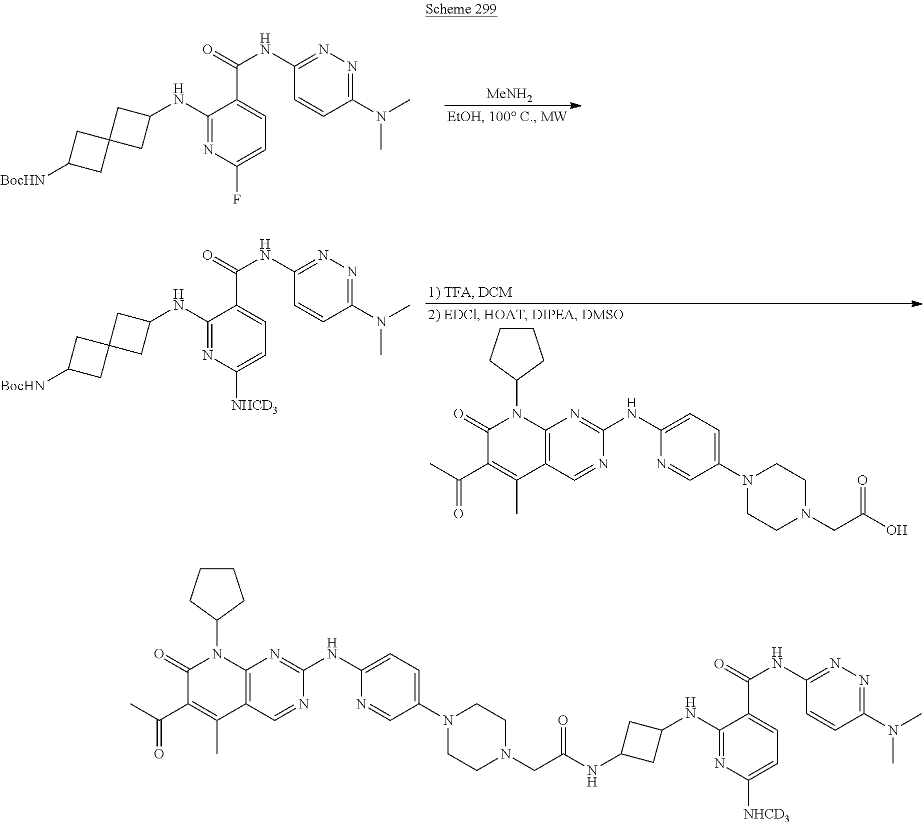

CPD-241 was synthesized following the standard procedures for preparing CPD-144 (12 mg, 19% yield over 3 steps) as a yellow solid. MS (ESI) m/z=887.7 [M+H]⁺.

### Example 300. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(methylamino)nicotinamide (CPD-242)

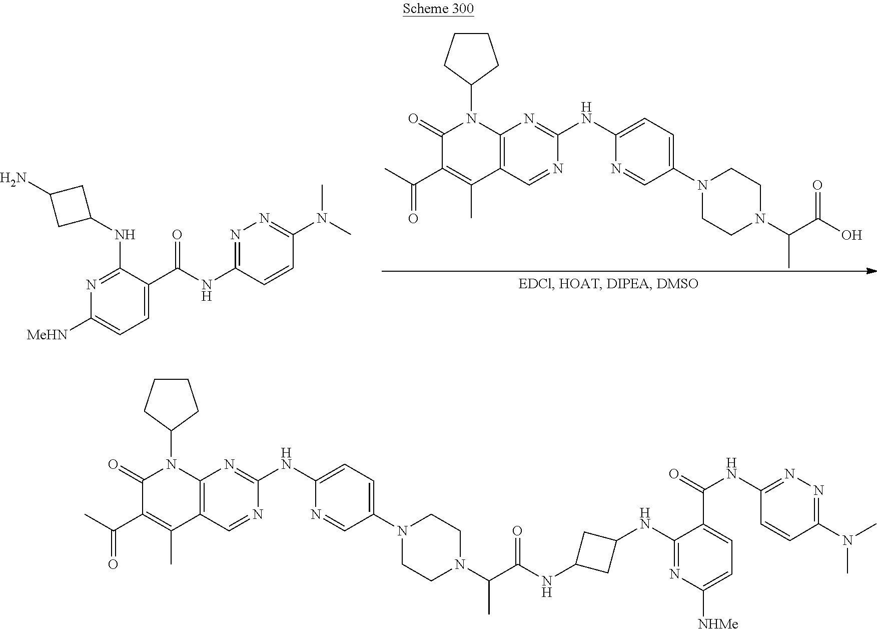

CPD-242 was synthesized following the standard procedures for preparing CPD-143 (13.8 mg, 33% yield) as a yellow solid. MS (ESI) m/z=858.6 [M+H]⁺.

### Example 301. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2-methylpropanamido)cyclobutyl)amino)-N-(6-(dimethylamino)pyridazin-3-yl)-6-(methylamino)nicotinamide (CPD-243)

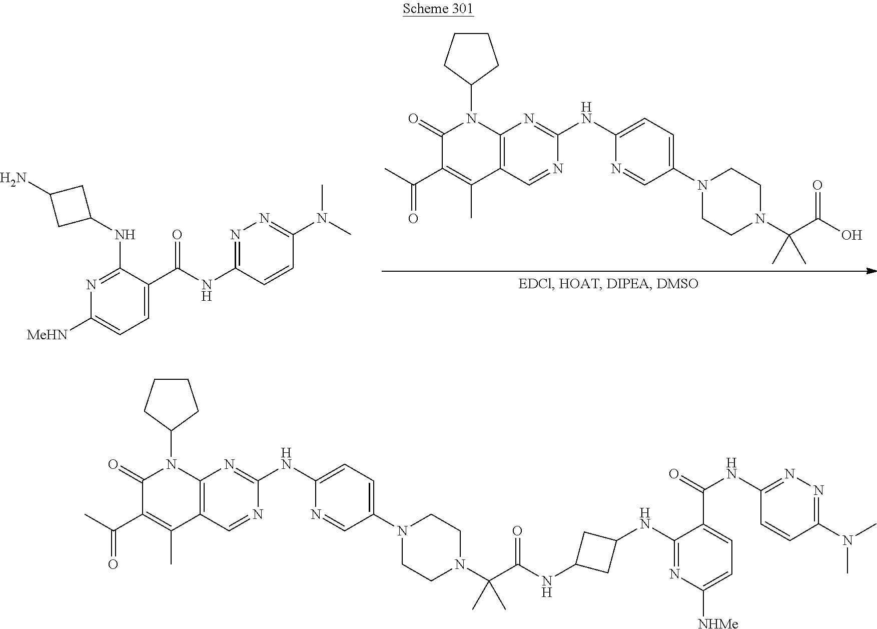

CPD-243 was synthesized following the standard procedures for preparing CPD-143 (6.7 mg, 16% yield) as a yellow solid. MS (ESI) m/z=872.6 [M+H]⁺.

### Example 302. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-((methyl-d3)amino)nicotinamide (CPD-244)

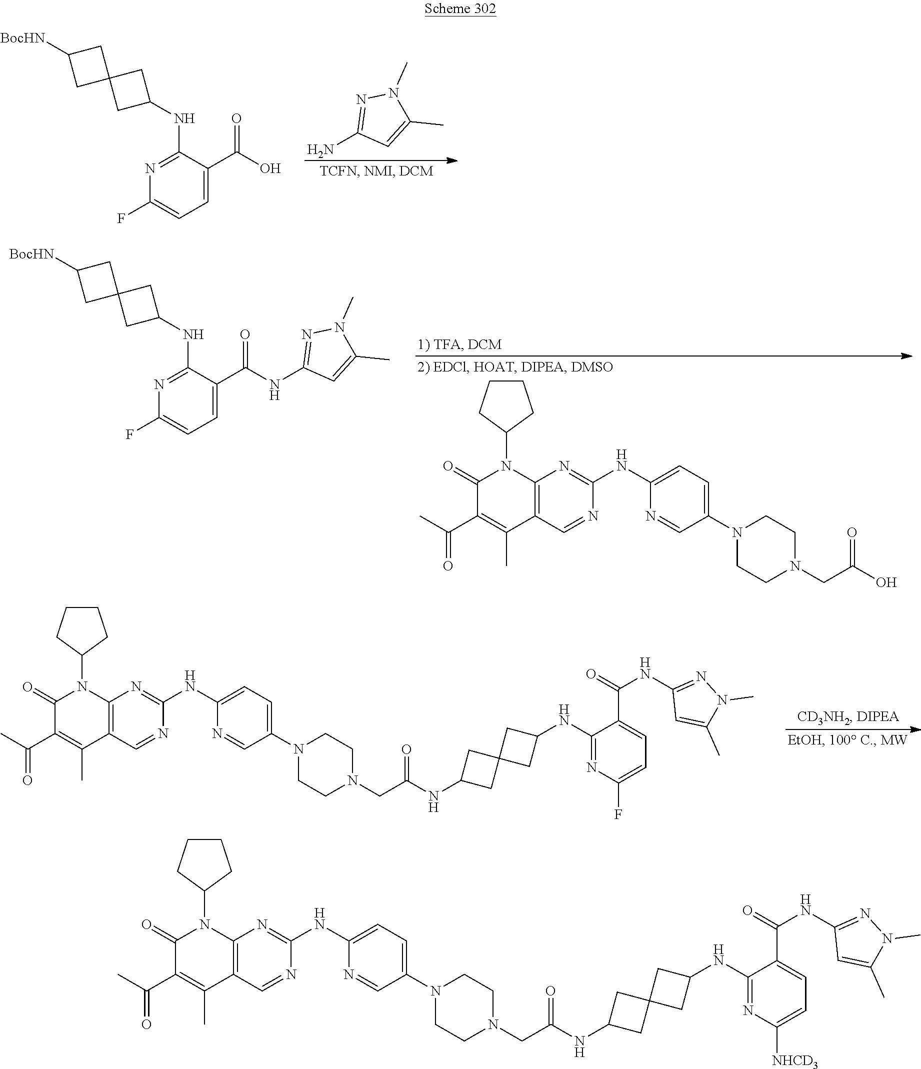

CPD-244 was synthesized following the standard procedures for preparing CPD-226 (6.9 mg, 2% yield over 4 steps) as a yellow solid. MS (ESI) m/z=860.7 [M+H]⁺.

### Example 303. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-((methyl-d₃)amino)nicotinamide (CPD-245)

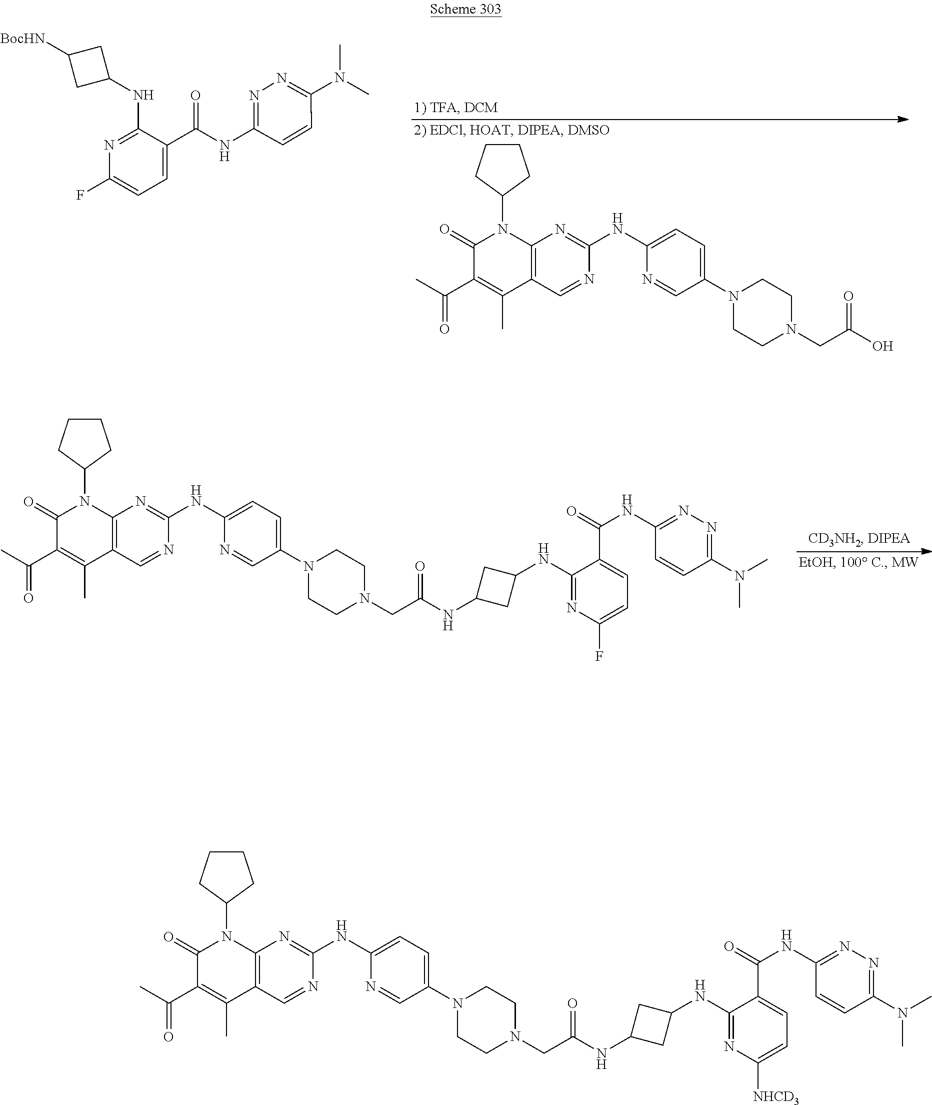

CPD-245 was synthesized following the standard procedures for preparing CPD-226 (5.0 mg, 17% yield over 3 steps) as a yellow solid. MS (ESI) m/z=847.7 [M+H]⁺.

### Example 304. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(methylamino)-N-(5-methyloxazol-2-yl)nicotinamide (CPD-246)

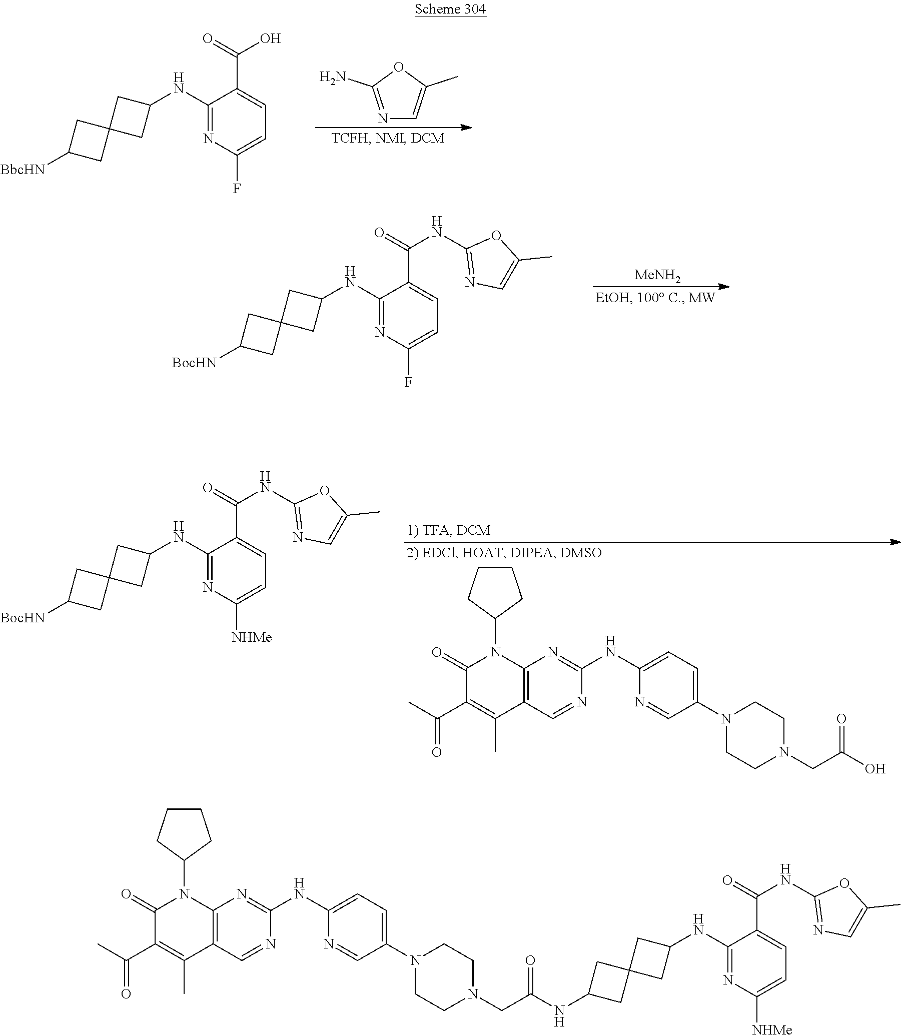

CPD-246 was synthesized following the standard procedures for preparing CPD-144 (1.2 mg, 1% yield over 4 steps) as a yellow solid. MS (ESI) m/z=844.7 [M+H]⁺.

### Example 305. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(4,5-dimethyloxazol-2-yl)-6-(methylamino)nicotinamide (CPD-247)

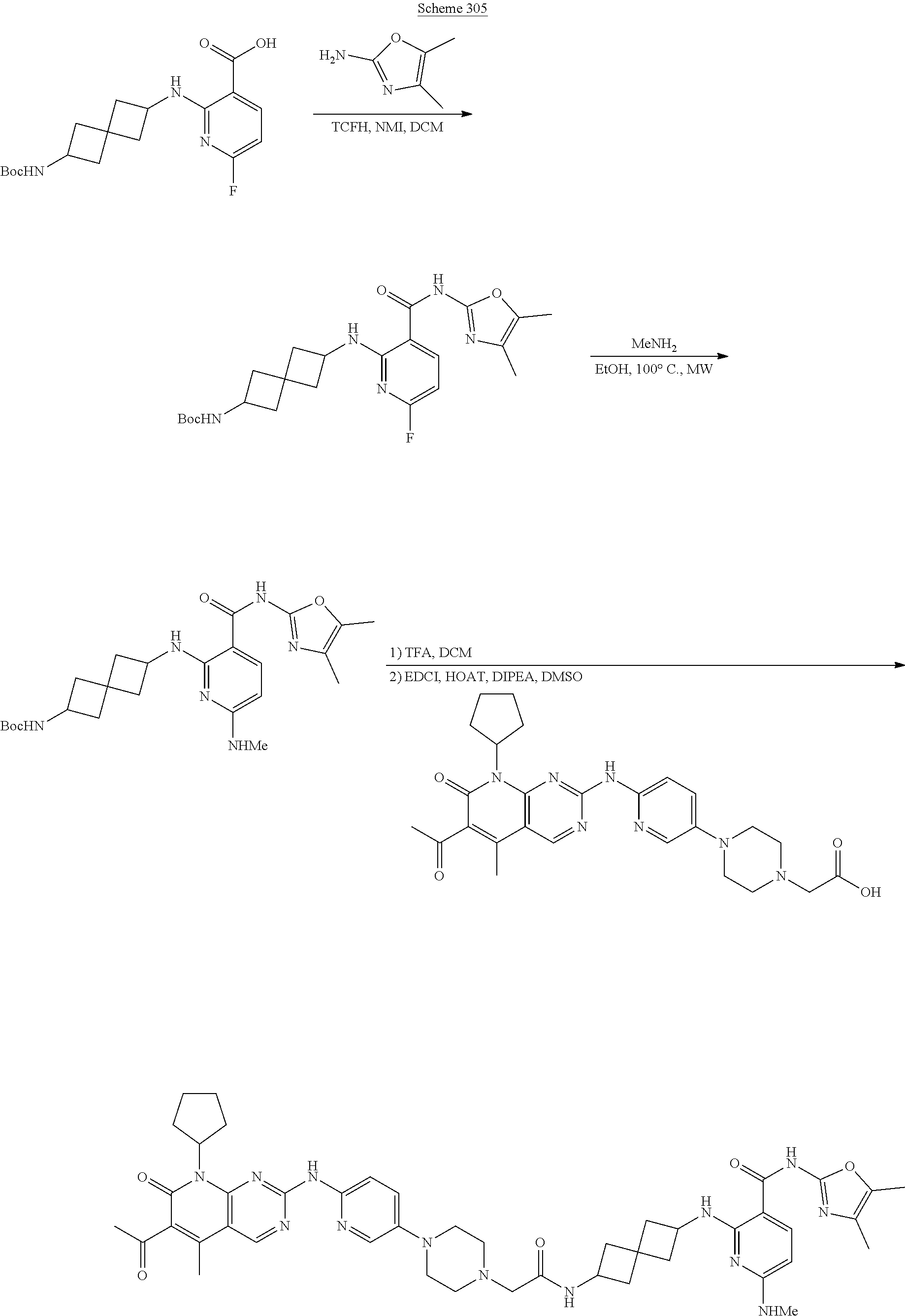

CPD-247 was synthesized following the standard procedures for preparing CPD-144 (0.95 mg, 1% yield over 4 steps) as a yellow solid. MS (ESI) m/z=858.6 [M+H]⁺.

### Example 306. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)azetidin-3-yl)amino)-N-(6-(dimethylamino)pyridazin-3-yl)-6-((methyl-d₃)amino)nicotinamide (CPD-248)

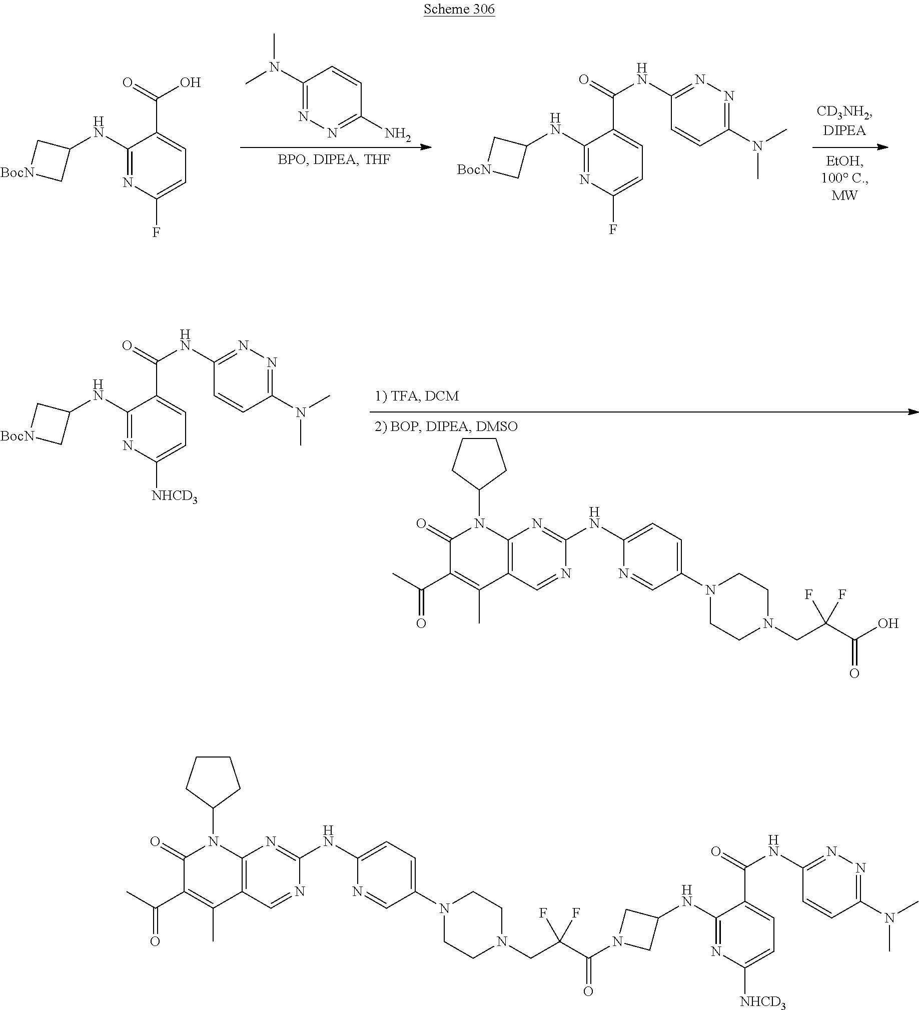

CPD-248 was synthesized following the standard procedures for preparing CPD-145 (9.0 mg, 13% yield over 4 steps) as a yellow solid. MS (ESI) m/z=883.6 [M+H]⁺.

### Example 307. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-2,2-difluoropropanoyl)-2-azaspiro[3.3]heptan-6-yl)amino)-N-(6-(dimethylamino)pyridazin-3-yl)-6-((methyl-d3)amino)nicotinamide (CPD-249)

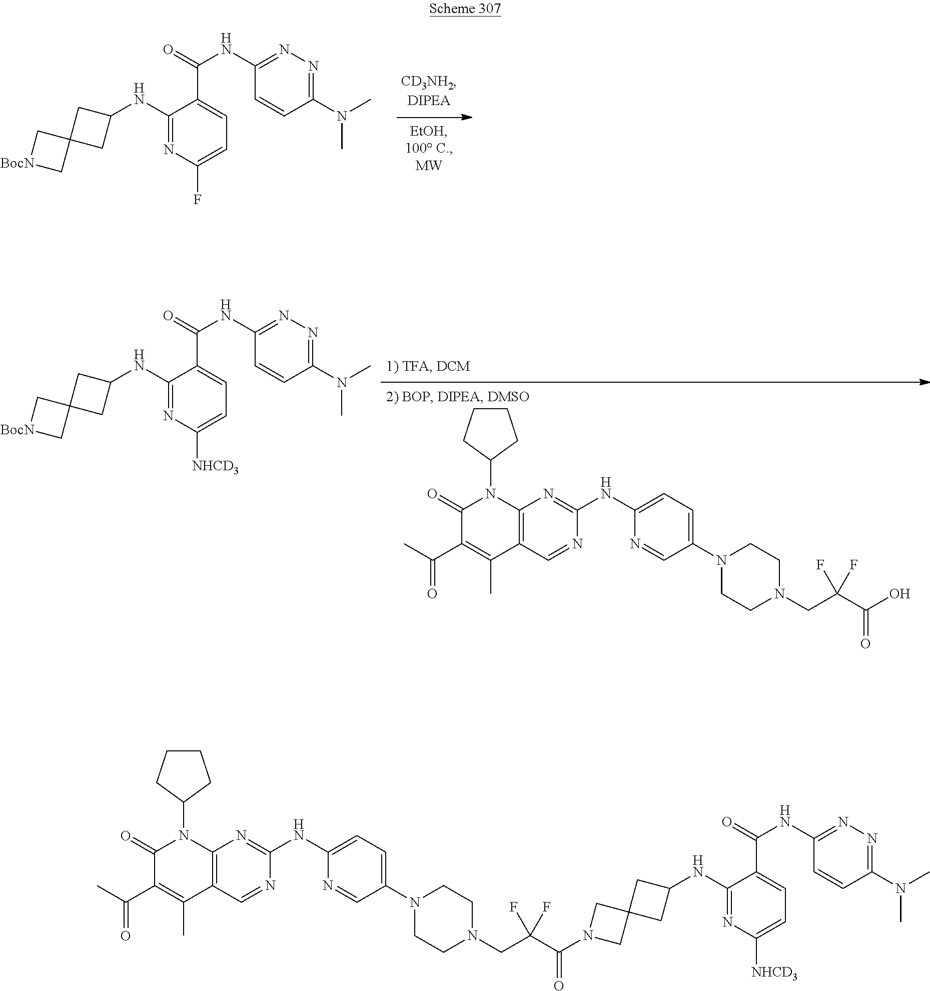

CPD-249 was synthesized following the standard procedures for preparing CPD-144 (8.0 mg, 20% yield over 3 steps) as a yellow solid. MS (ESI) m/z=923.7 [M+H]⁺.

### Example 308. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-((methyl-d3)amino)nicotinamide (CPD-250)

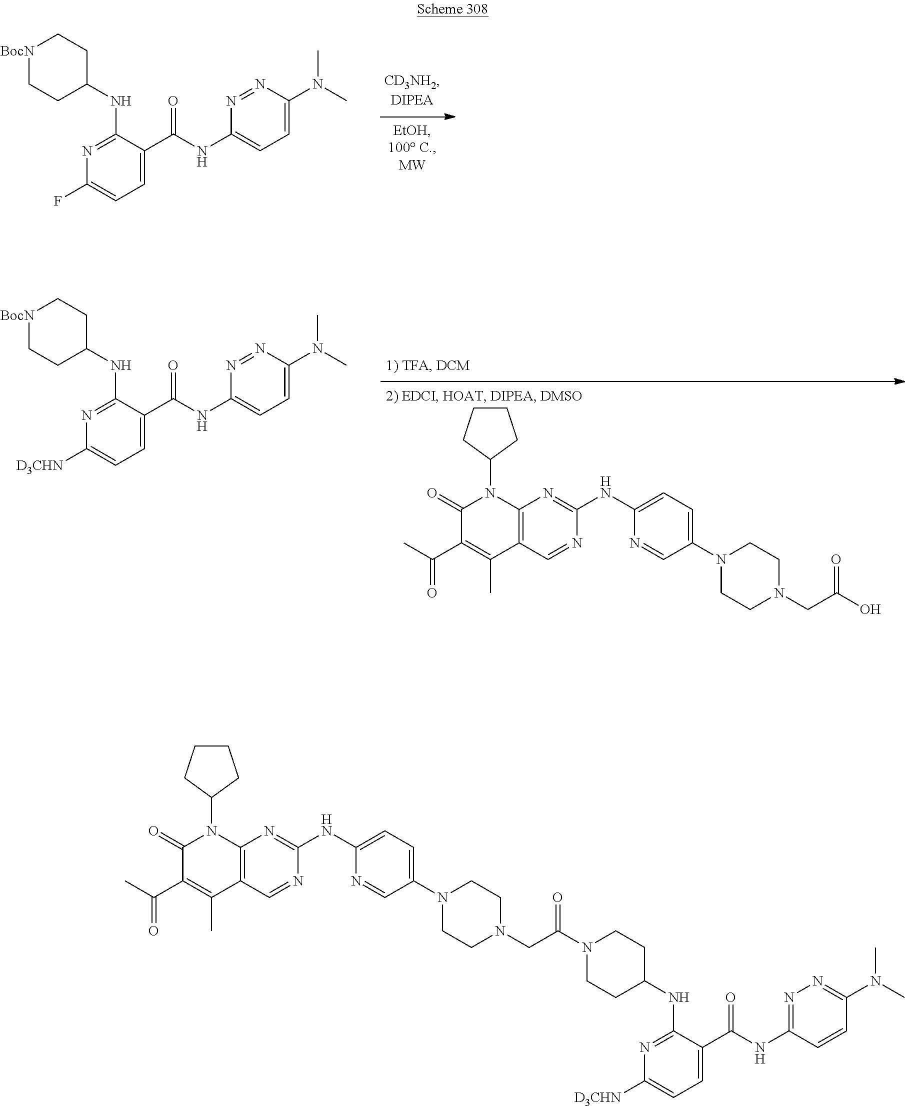

CPD-250 was synthesized following the standard procedures for preparing CPD-144 (2.8 mg, 4% yield over 3 steps) as a yellow solid. MS (ESI) m/z=861.6 [M+H]⁺.

### Example 309. 6-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-1H-pyrrolo[2,3-b]pyridine-5-carboxamide (CPD-251)

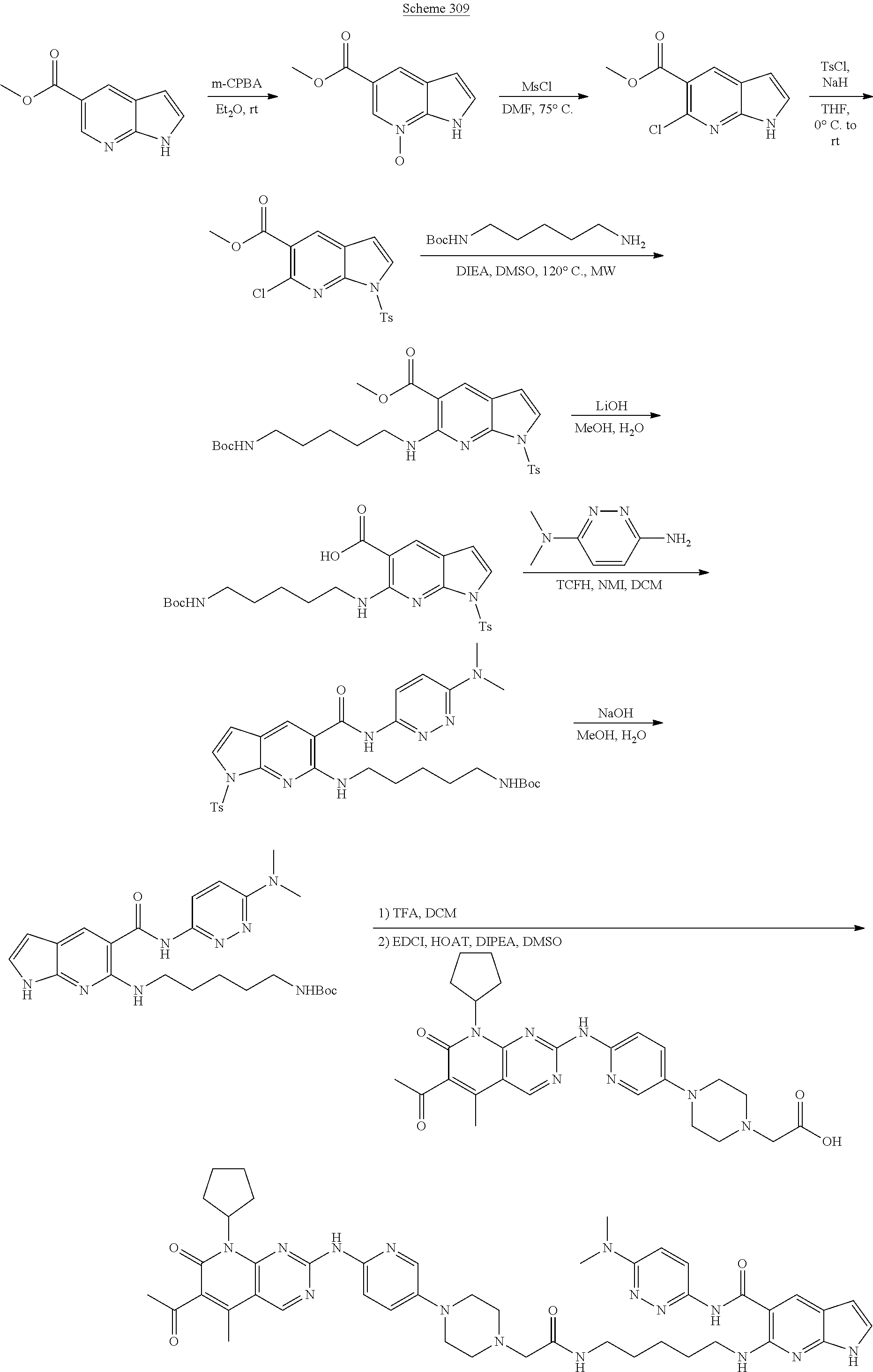

### Step 1. Synthesis of 5-(methoxycarbonyl)-1H-pyrrolo[2,3-b]pyridine 7-oxide

To a solution of methyl 1H-pyrrolo[2,3-b]pyridine-5-carboxylate (3 g, 17.0 mmol) in Et₂O (150 mL) was added m-CPBA (4.1 g, 20.4 mmol). The mixture was stirred at rt for 5 h. The mixture was filtered to get solid product (2.4 g, 73% yield) as a brown solid. MS (ESI) m/z=193.3 [M+H]⁺.

### Step 2. Synthesis of methyl 6-chloro-1H-pyrrolo[2,3-b]pyridine-5-carboxylate

To a solution of 5-(methoxycarbonyl)-1H-pyrrolo[2,3-b]pyridine 7-oxide (2.4 g, 12.5 mmol) in DMF (50 mL) was added MsCl (4.29 g, 37.5 mmol). The mixture was stirred at 75′C for 1 h. The mixture was quenched with water and extracted with EtOAc. The organic phase was washed with water and brine, dried over Na₂SO₄, filtered and concentrated. The residue was purified by column chromatography on silica gel (petroleum ether/ethyl acetate=5:1) to afford the title compound (400 mg, 15% yield) as a white solid. MS (ESI) m/z=211.1 [M+H]⁺.

### Step 3. Synthesis of methyl 6-chloro-1-tosyl-1H-pyrrolo[2,3-b]pyridine-5-carboxylate

To a solution of methyl 6-chloro-1H-pyrrolo[2,3-b]pyridine-5-carboxylate (400 mg, 1.9 mmol) in THF (40 mL) was added sodium hydride (60% dispersion in paraffin liquid, 91 mg, 2.27 mmol) under N₂ at 0 [symbol] C. After stirring at 0 [symbol] for 0.5 h, TsCl (724 mg, 3.7 mmol) was added to the mixture. The mixture was stirred at rt for 16 h. The reaction mixture was subsequently quenched with saturated NH₄Cl solution and extracted with EtOAc. The combined organic layers were washed brine, dried over anhydrous Na₂SO₄, filtered and concentrated. The residue was purified by column chromatography on silica gel (petroleum ether/ethyl acetate=3:1) to provide the desired compound (400 mg, 58% yield) as a white solid. MS (ESI) m/z=365.2 [M+H]⁺.

### Step 4. Synthesis of methyl 6-((5-((tert-butoxycarbonyl)amino)pentyl)amino)-1-tosyl-1H-pyrrolo[2,3-b]pyridine-5-carboxylate

To a solution of methyl 6-chloro-1-tosyl-1H-pyrrolo[2,3-b]pyridine-5-carboxylate (400 mg, 1.12 mmol) in DMSO (12 mL) were added DIEA (283 mg, 2.24 mmol) and tert-butyl (5-aminopentyl)carbamate (221 mg, 1.12 mmol). The mixture was stirred at 120° C. under microwave irradiation for 2 h. The mixture was quenched with water and extracted with EtOAc. The combined organic phase was washed with brine, dried over Na₂SO₄, filtered and concentrated. The residue was purified by reverse-phase chromatography to afford the desired compound (175 mg, 30% yield) as a white solid. MS (ESI) m/z=531.4 [M+H]⁺.

### Step 5. Synthesis of 6-((5-((tert-butoxycarbonyl)amino)pentyl)amino)-1-tosyl-1H-pyrrolo[2,3-b]pyridine-5-carboxylic acid

A mixture of methyl 6-((5-((tert-butoxycarbonyl)amino)pentyl)amino)-1-tosyl-1H-pyrrolo[2,3-b]pyridine-5-carboxylate (50 mg, 0.09 mmol) and LiOH (22 mg, 0.9 mmol) in MeOH (5 mL) and H₂O (1 mL) was stirred at rt for 2 h. The resulting mixture was concentrated and purified by reverse-phase chromatography to give the desired product (42 mg, 70% yield) as a white solid. MS (ESI) m/z=517.4 [M+H]⁺.

### Step 6. Synthesis of tert-butyl (5-((5-((6-(dimethylamino)pyridazin-3-yl)carbamoyl)-1-tosyl-1H-pyrrolo[2,3-b]pyridin-6-yl)amino)pentyl)carbamate

A mixture of 6-((5-((tert-butoxycarbonyl)amino)pentyl)amino)-1-tosyl-1H-pyrrolo[2,3-b]pyridine-5-carboxylic acid (36 mg, 0.07 mmol), N³,N³-dimethylpyridazine-3,6-diamine (14 mg, 0.1 mmol), TCFH (39 mg, 0.14 mmol) and NMI (28 mg, 0.3 mmol) in dichloromethane (5 mL) was stirred at rt for 2 h. The reaction mixture was poured into water (20 mL) and extracted with ethyl acetate (3×20 mL). The combined organic layers were washed with saturated brine (20 mL), dried over anhydrous Na₂SO₄, filtered, and evaporated under reduced pressure. The resulting residue was purified by silica gel flash chromatography to give the desired product (35 mg, 46% yield) as a colorless oil. MS (ESI) m/z=637.7 [M+H]⁺.

### Step 7. Synthesis of tert-butyl (5-((5-((6-(dimethylamino)pyridazin-3-yl)carbamoyl)-1H-pyrrolo[2,3-b]pyridin-6-yl)amino)pentyl)carbamate

A mixture of tert-butyl (5-((5-((6-(dimethylamino)pyridazin-3-yl)carbamoyl)-1-tosyl-1H-pyrrolo[2,3-b]pyridin-6-yl)amino)pentyl)carbamate (59 mg, 0.09 mmol) and NaOH (18 mg, 0.45 mmol) in MeOH (1 mL) and H₂O (1 mL) was stirred at 100° C. for 2 h. The resulting mixture was concentrated and purified by reverse-phase chromatography to give the desired product (28 mg, 37% yield) as a white solid. MS (ESI) m/z=483.5 [M+H]⁺.

### Step 8. Synthesis of 6-((5-aminopentyl)amino)-N-(6-(dimethylamino)pyridazin-3-yl)-1H-pyrrolo[2,3-b]pyridine-5-carboxamide

To a solution of tert-butyl (5-((5-((6-(dimethylamino)pyridazin-3-yl)carbamoyl)-1H-pyrrolo[2,3-b]pyridin-6-yl)amino)pentyl)carbamate (28 mg, 0.06 mmol) in dichloromethane (1 mL) was added TFA (0.5 mL). The mixture was stirred at rt for 1 h. The solution was concentrated under reduced pressure to provide the crude product (28 mg, 52% yield) as a yellow oil. This compound was used directly in the next step without further purification. MS (ESI) m/z=383.4 [M+H]⁺.

### Step 9. Synthesis of 6-((5-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)pentyl)amino)-N-(6-(dimethylamino)pyridazin-3-yl)-1H-pyrrolo[2,3-b]pyridine-5-carboxamide

A mixture of 6-((5-aminopentyl)amino)-N-(6-(dimethylamino)pyridazin-3-yl)-1H-pyrrolo[2,3-b]pyridine-5-carboxamide (12 mg, 0.01 mmol), 5-((3-((6-(dimethylamino)pyridazin-3-yl)carbamoyl)-6-(methylamino)pyridin-2-yl)amino)pentanoic acid (6.2 mg, 0.01 mmol), EDCI (3.5 mg, 0.02 mmol), HOAt (2.7 mg, 0.02 mmol) and DIPEA (8 mg, 0.06 mmol) in DMSO (2 mL) was stirred at rt for 16 h. The reaction mixture was purified by reverse-phase chromatography to give the desired product (1.4 mg, 11% yield) as a light-yellow solid. MS (ESI) m/z=870.5 [M+H]⁺.

### Example 310. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-cyclopropylpyridin-2-yl)-6-((methyl-d₃)amino)nicotinamide (CPD-252)

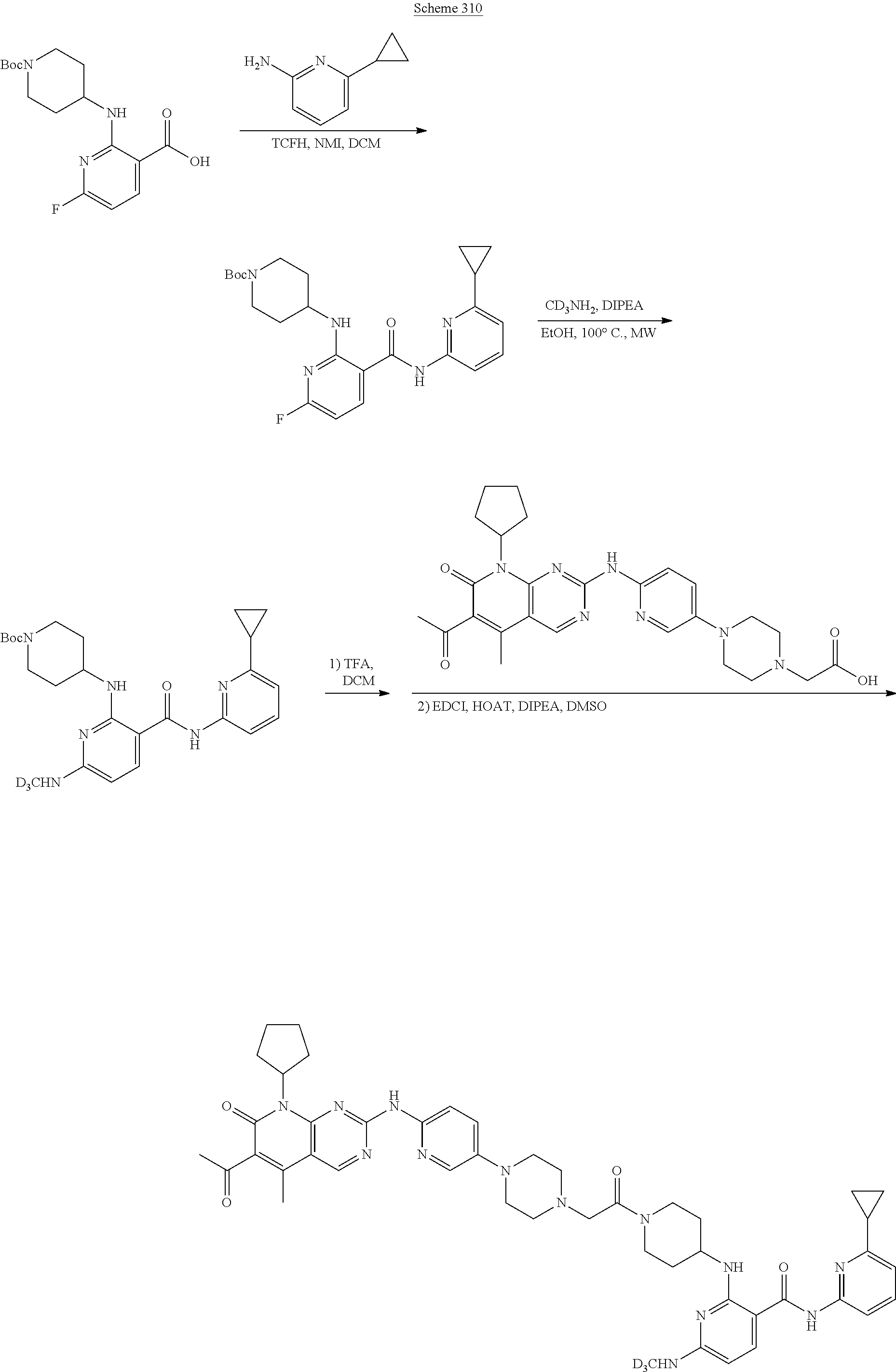

CPD-252 was synthesized following the standard procedures for preparing CPD-144 (15 mg, 30% yield over 4 steps) as a yellow solid. MS (ESI) m/z=857.8 [M+H]⁺.

### Example 311. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-(isopropylamino)nicotinamide (CPD-253)

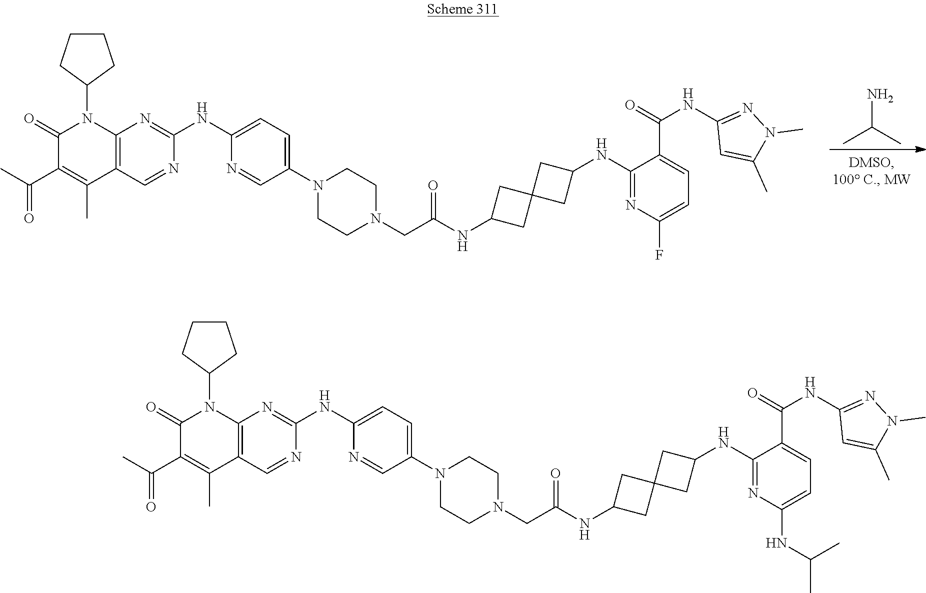

To a mixture of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1,5-dimethyl-1H-pyrazol-3-yl)-6-fluoronicotinamide (6.5 mg, 0.008 mmol) in DMSO (1 mL) was added propan-2-amine (4.5 mg, 0.08 mmol). The reaction mixture was stirred at 100° C. for 1 h under microwave irradiation. After cooling down to rt, the mixture was purified by reverse-phase chromatography to give the desired product (5.6 mg, 72% yield) as a white solid. MS (ESI) m/z=885.7 [M+H]⁺.

### Example 312. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-(methylamino)nicotinamide (CPD-254)

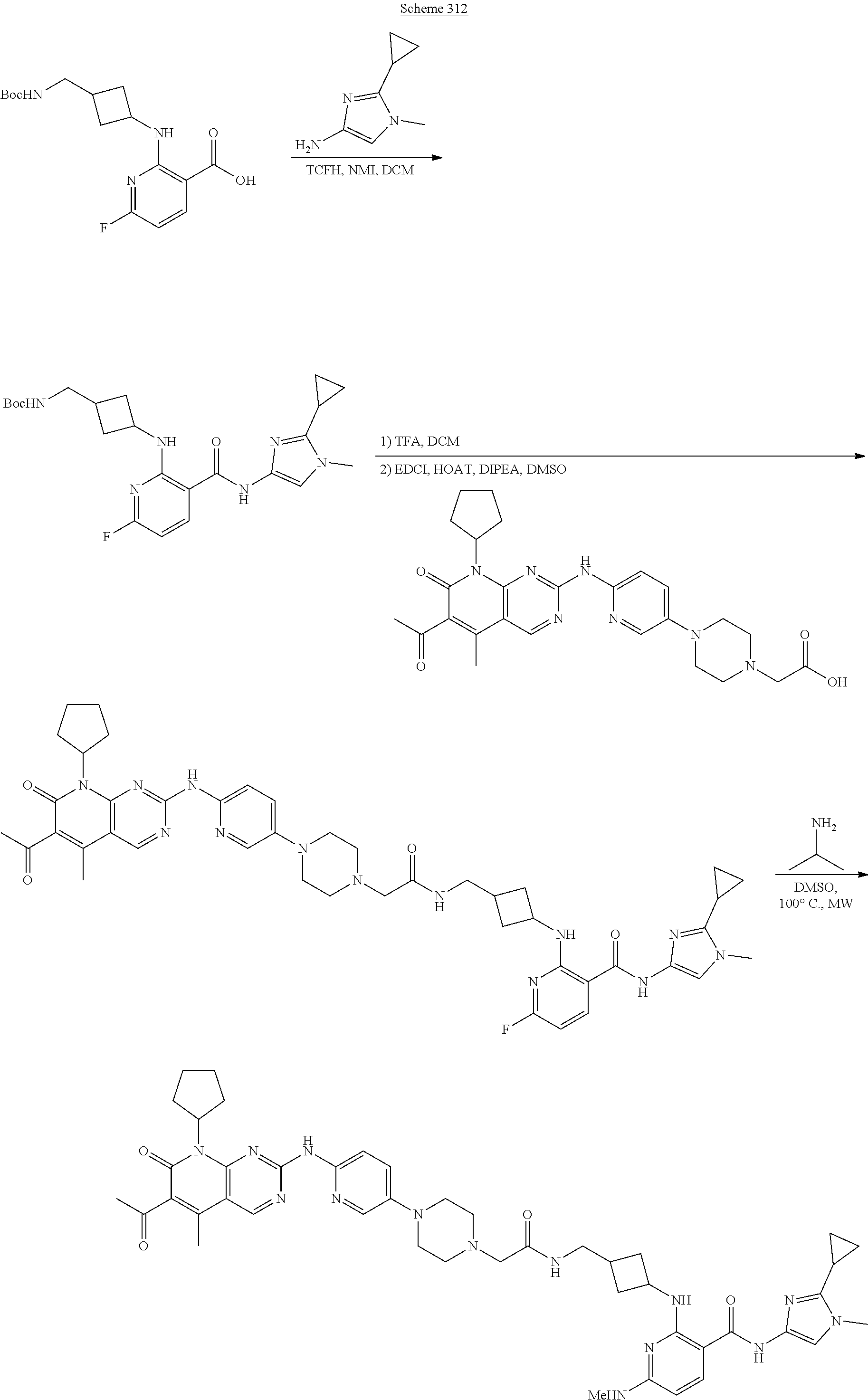

CPD-254 was synthesized following the standard procedures for preparing CPD-144 and CPD-253 (1.8 mg, 5% yield over 4 steps) as a yellow solid. MS (ESI) m/z=857.8 [M+H]⁺.

### Example 313. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-chloro-N-(5-methylisoxazol-3-yl)benzamide (CPD-259)

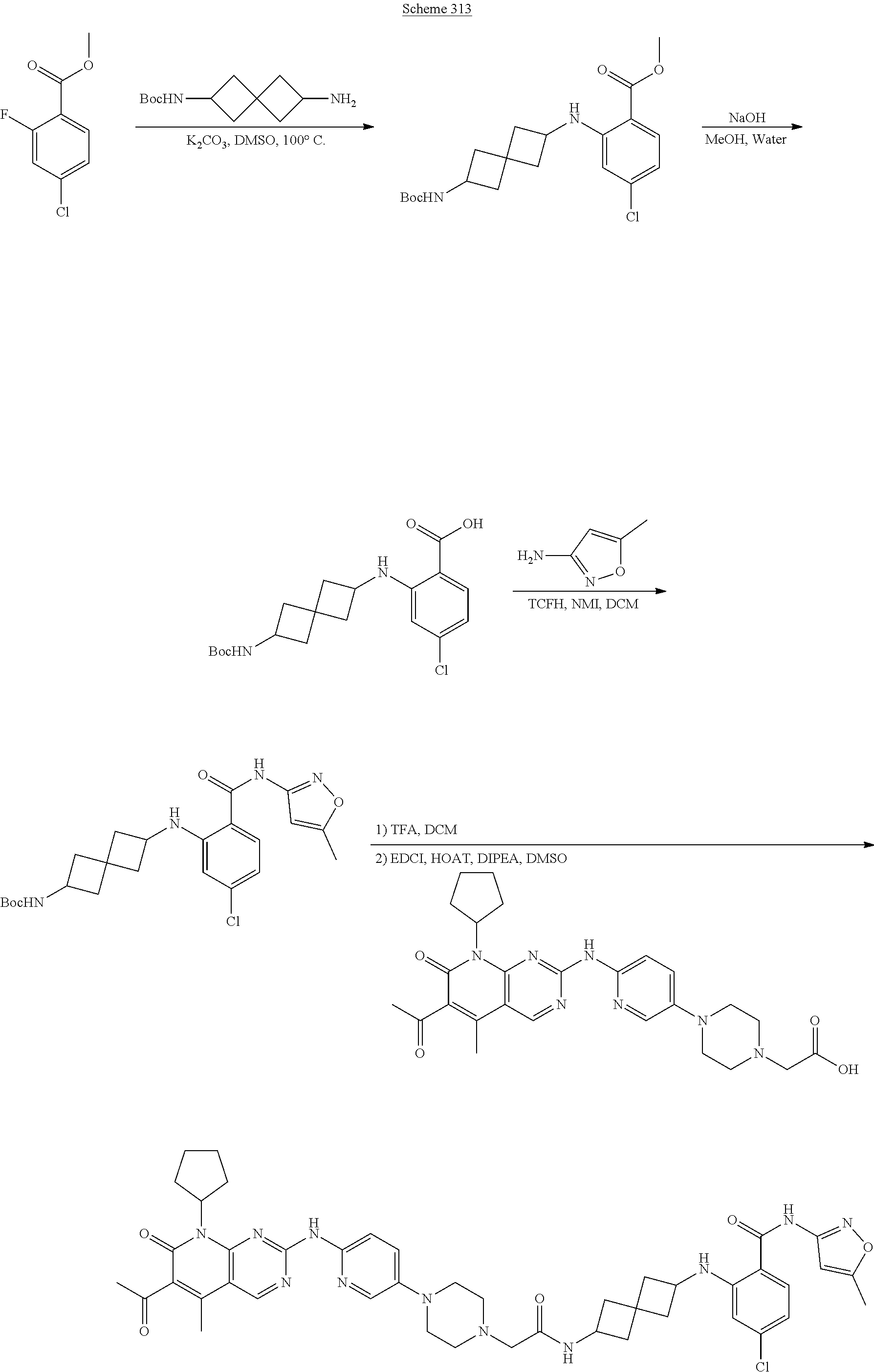

CPD-259 was synthesized following the standard procedures for preparing CPD-255 (11.5 mg, 8% yield over 5 steps) as a yellow solid. MS (ESI) m/z=848.5 [M+H]⁺.

### Example 314. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-fluoro-N-(5-methylisoxazol-3-yl)benzamide (CPD-260)

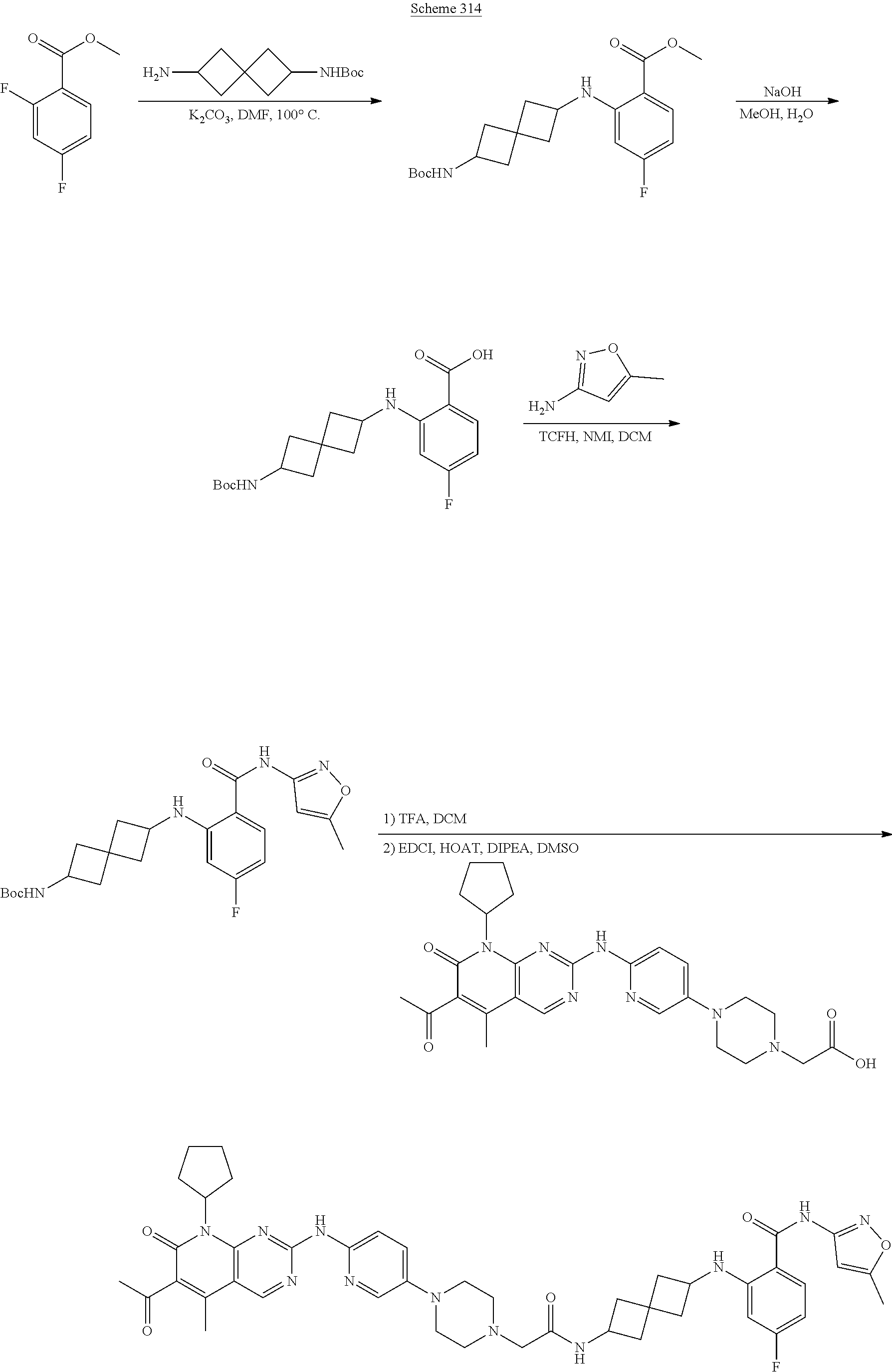

CPD-260 was synthesized following the standard procedures for preparing CPD-255 (4.0 mg, 2% yield over 5 steps) as a yellow solid. MS (ESI) m/z=832.7 [M+H]⁺.

### Example 315. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-4-(trifluoromethyl)benzamide (CPD-261)

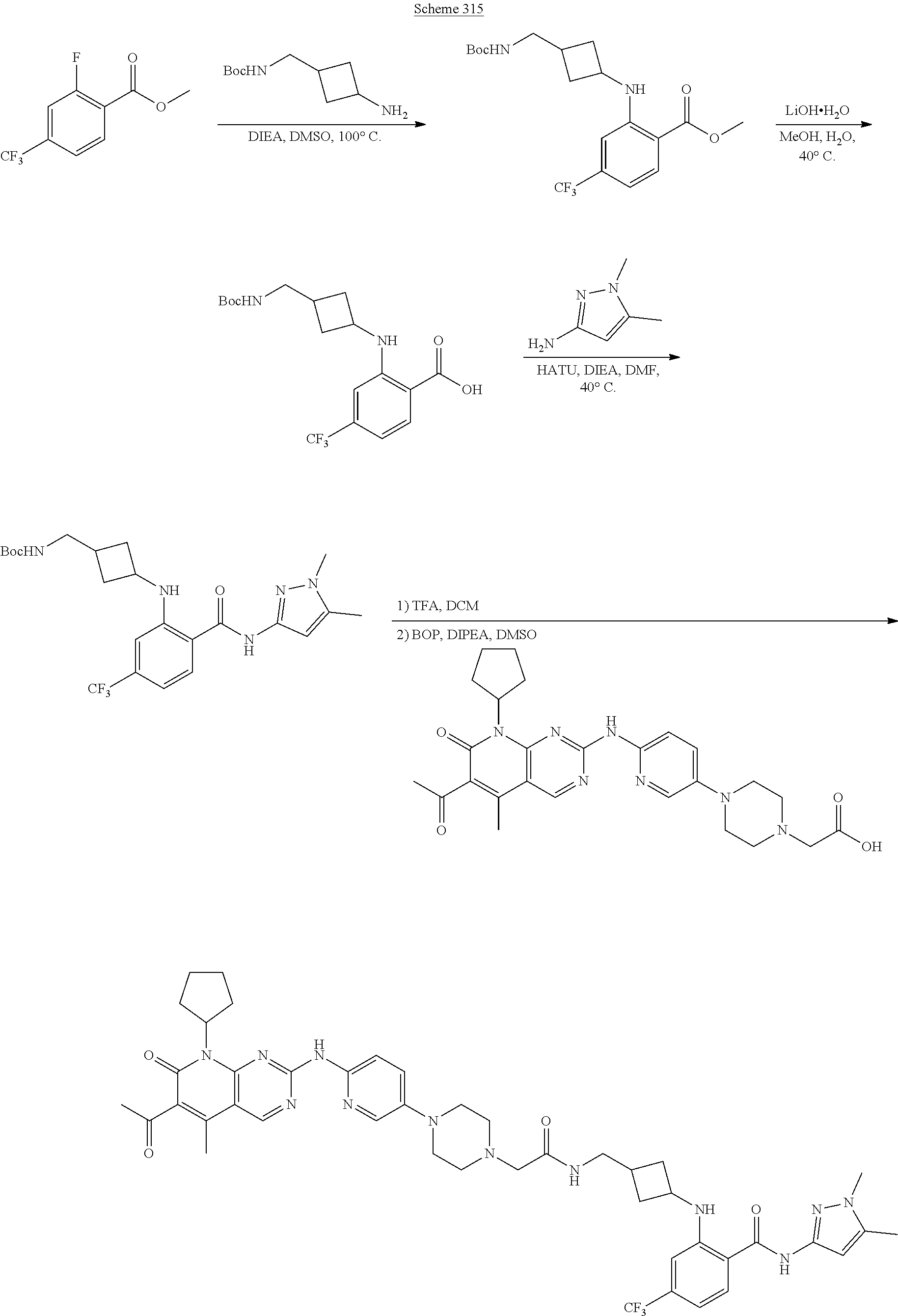

### Step 1. Synthesis of methyl 2-((3-(((tert-butoxycarbonyl)amino)methyl)cyclobutyl)amino)-4-(trifluoromethyl)benzoate

To a solution of tert-butyl N-[(3-aminocyclobutyl)methyl]carbamate (250 mg, 1.25 mmol) and methyl 2-fluoro-4-(trifluoromethyl)benzoate (338 mg, 1.52 mmol) in DMSO (3 mL) was added DIPEA (484 mg, 3.74 mmol). The mixture was stirred at 100° C. for 12 h. The reaction mixture was poured into H₂O (20 mL) and extracted with ethyl acetate (20 mL×3). The combined organic layers were washed with brine (30 mL), dried over Na₂SO₄, filtered and concentrated. The residue was purified by column chromatography on silica gel (petroleum ether/ethyl acetate=15:1 to 5:1) to provide the desired product (150 mg, 34% yield) as a white solid.

### Step 2. Synthesis of 2-((3-(((tert-butoxycarbonyl)amino)methyl)cyclobutyl)amino)-4-fluorobenzoic acid

To a solution of methyl 2-[[3-[(tert-butoxycarbonylamino)methyl]cyclobutyl]amino]-4-(trifluoromethyl)benzoate (150 mg, 372 μmol) in MeOH (3 mL) and H₂O (1 mL) was added LiOH·H₂O (40 mg, 953 μmol). The mixture was stirred at 40 [symbol] for 12 h. The reaction mixture was poured into aq. HCl (2 N, 20 mL) and extracted with ethyl acetate (20 mL×3). The combined organic layers were washed with brine (20 mL), dried over Na₂SO₄, filtered, and concentrated to provide the desired product (112 mg, 77% yield) as a white solid.

### Step 3. Synthesis of tert-butyl ((3-((2-((1,5-dimethyl-1H-pyrazol-3-yl)carbamoyl)-5-(trifluoromethyl)phenyl)amino)cyclobutyl)methyl)carbamate

To a solution of 2-[[3-[(tert-butoxycarbonylamino)methyl]cyclobutyl]amino]-4-(trifluoromethyl)benzoic acid (56 mg, 144 μmol) and 1,5-dimethylpyrazol-3-amine (56 mg, 503 μmol) in DMF (2 mL) were added DIPEA (56 mg, 433 μmol) and HATU (66 mg, 173 μmol). The mixture was stirred at 40° C. for 12 h. The reaction mixture was poured into water (50 mL) and extracted with ethyl acetate (25 mL×3). The combined organic layers were washed with brine (50 mL), dried over Na₂SO₄, filtered and concentrated. The residue was purified by prep-TLC (petroleum ether/ethyl acetate=1:1) to provide the desired product (67 mg, 48% yield) as a white solid. ¹HNMR (400 MHz, CDCl₃) δ 8.43 (s, 11H), 8.26-8.08 (m, 1H), 7.69 (s, 1H), 7.49 (d, J=8.4 Hz, 1H), 6.61 (d, J=8.8 Hz, 1H), 6.52 (s, 1H), 4.56 (s, 1H), 4.10-3.76 (m, 1H), 3.67 (s, 3H), 3.36-3.10 (m, 2H), 2.65-2.54 (m, 1H), 2.53-2.43 (m, 0.4H), 2.29 (s, 3H), 2.27-2.20 (m, 1H), 2.15-2.07 (m, 0.6H), 1.73-1.60 (m, 2H), 1.50-1.40 (m, 9H). MS (ESI) m/z=481.2 [M+H]⁺.

### Step 4. Synthesis of 2-((3-(aminomethyl)cyclobutyl)amino)-N-(1,5-dimethyl-1H-pyrazol-3-yl)-4-(trifluoromethyl)benzamide

To a solution of tert-butyl ((3-((2-((1,5-dimethyl-1H-pyrazol-3-yl)carbamoyl)-5-(trifluoromethyl)phenyl)amino)cyclobutyl)methyl)carbamate (30 mg, 62.30 mol) in dichloromethane (3 mL) was added TFA (1 mL). After the mixture was stirred at rt for 1 h, it was evaporated under reduced pressure to get the desired product (22 mg, 92% yield) as a light-yellow solid, which was used directly to the next step without further purification. MS(ESI) m/z=382.3[M+H]⁺.

### Step 5. Synthesis of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-4-(trifluoromethyl)benzamide

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (29 mg, 57.68 μmol) in DMSO (3 mL) were added 2-((3-(aminomethyl)cyclobutyl)amino)-N-(1,5-dimethyl-1H-pyrazol-3-yl)-4-(trifluoromethyl)benzamide (22 mg, 57.68 μmol), DIPEA (37 mg, 0.29 mmol) and BOP (51 mg, 0.12 mmol). The mixture was stirred at 40° C. for 3 h. The reaction mixture was poured into water (10 mL) and extracted with ethyl acetate (2×20 mL). The combined organic layers were washed with saturated brine (10 mL), dried over anhydrous sodium sulfate, filtered, and evaporated under reduced pressure. The resulting residue was purified by reverse-phase chromatography to give the desired product (20 mg, 40% yield) as a yellow solid. MS(ESI) m/z=869.5 [M+H]⁺.

### Example 316. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-5-(trifluoromethyl)benzamide (CPD-262)

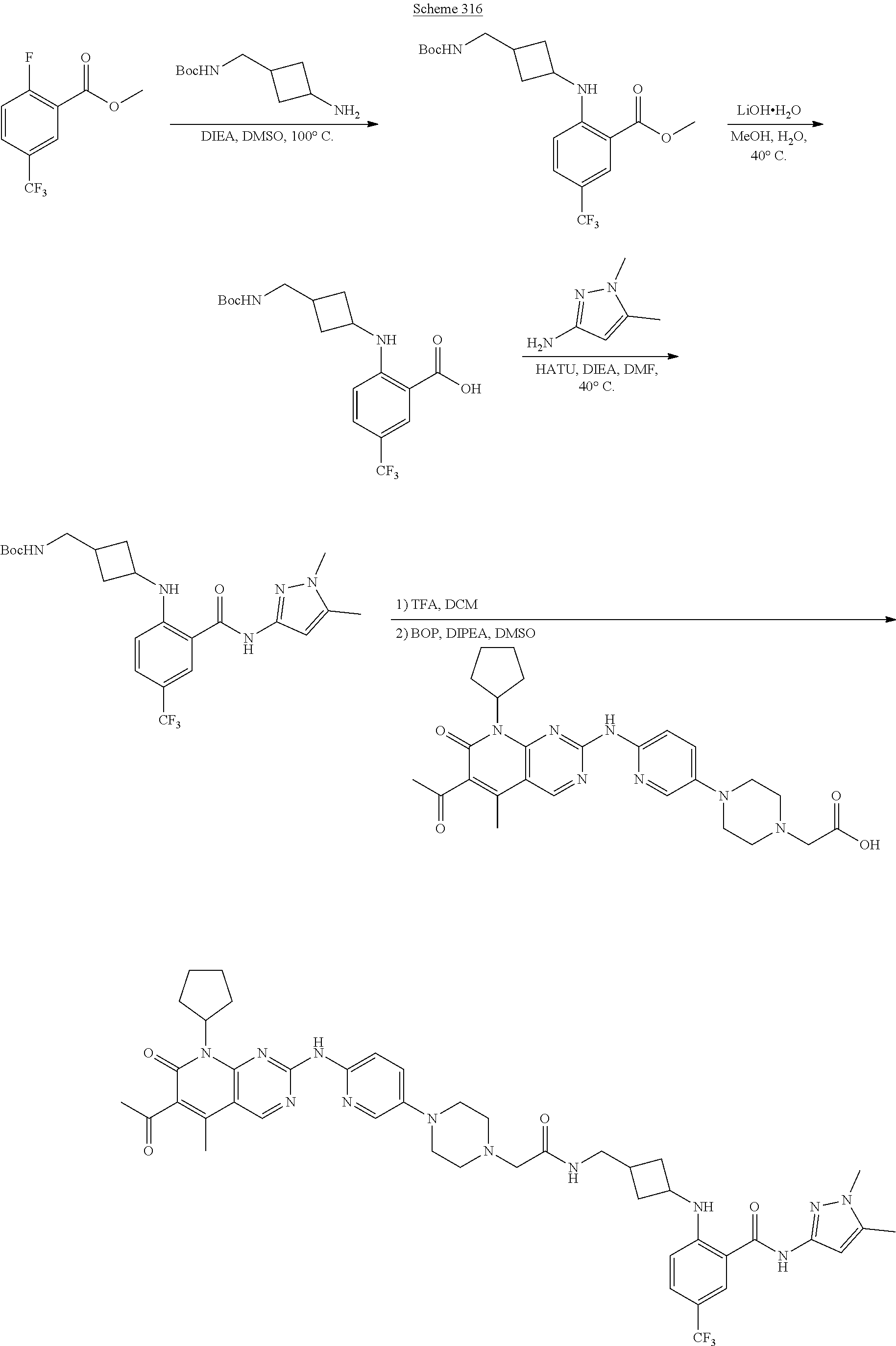

CPD-262 was synthesized following the standard procedures for preparing CPD-261 (10.0 mg, 3% yield over 5 steps) as a yellow solid. MS (ESI) m/z=869.6 [M+H]⁺.

### Example 317. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-5-fluorobenzamide (CPD-263)

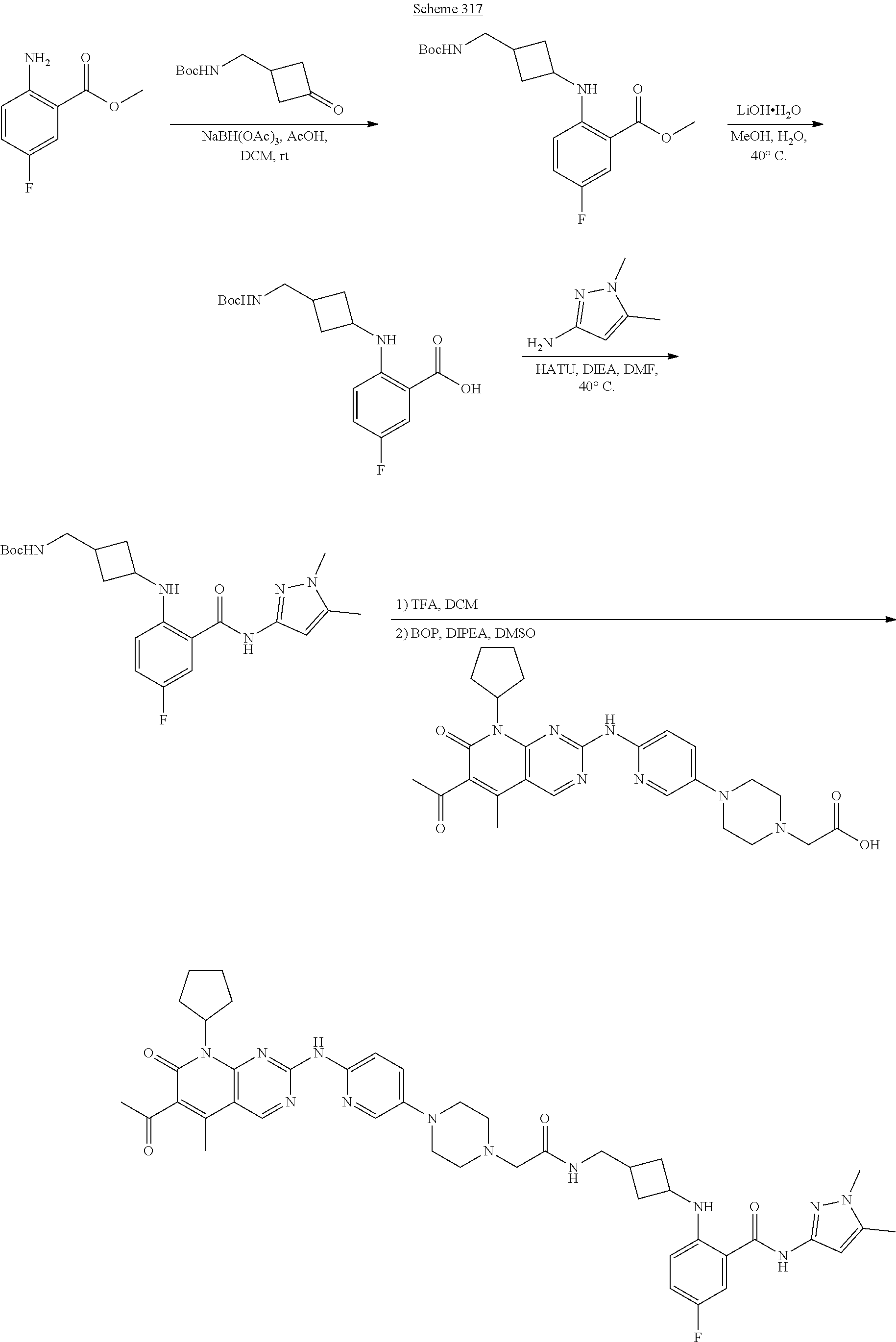

### Step 1. Synthesis of methyl 2-((3-(((tert-butoxycarbonyl)amino)methyl)cyclobutyl)amino)-5-fluorobenzoate

To a solution of methyl 2-amino-5-fluoro-benzoate (300 mg, 1.77 mmol) and tert-butyl N-[(3-oxocyclobutyl)methyl]carbamate (354 mg, 1.78 mmol) in dichloromethane (3 mL) were added AcOH (639 mg, 10.6 mmol) and NaBH(OAc)₃ (451 mg, 2.13 mmol). The mixture was stirred at rt for 12 h. The reaction mixture was poured into saturated aq. NH₄Cl solution (20 mL) and extracted with ethyl acetate (30 mL×3). The combined organic layers were washed with brine (100 mL), dried over Na₂SO₄, filtered and concentrated. The crude product was purified by pre-HPLC to provide the desired product (100 mg, 16% yield) as a yellow oil. MS (ESI) m/z=353.5 [M+H]⁺.

The remaining steps were performed following the standard procedures for preparing CPD-261 to provide the desired product (9.3 mg, 30% yield over 4 steps) as a yellow solid. MS (ESI) m/z=819.6 [M+H]⁺.

### Example 318. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-4-fluorobenzamide (CPD-264)

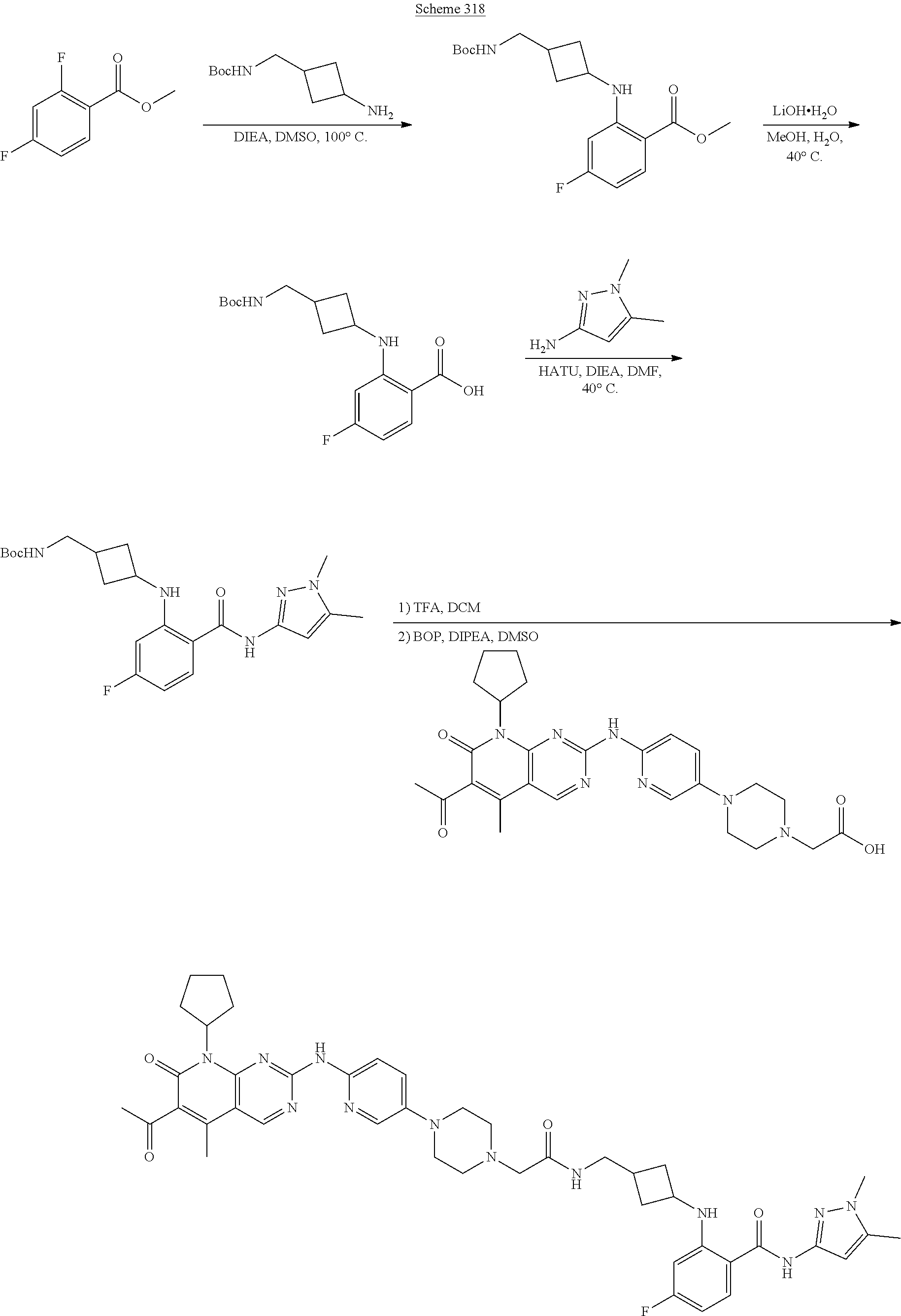

CPD-264 was synthesized following the standard procedures for preparing CPD-261 (6.4 mg, 3% yield over 5 steps) as a yellow solid. MS (ESI) m/z=819.6 [M+H]⁺.

### Example 319. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-fluorobenzamide (CPD-265)

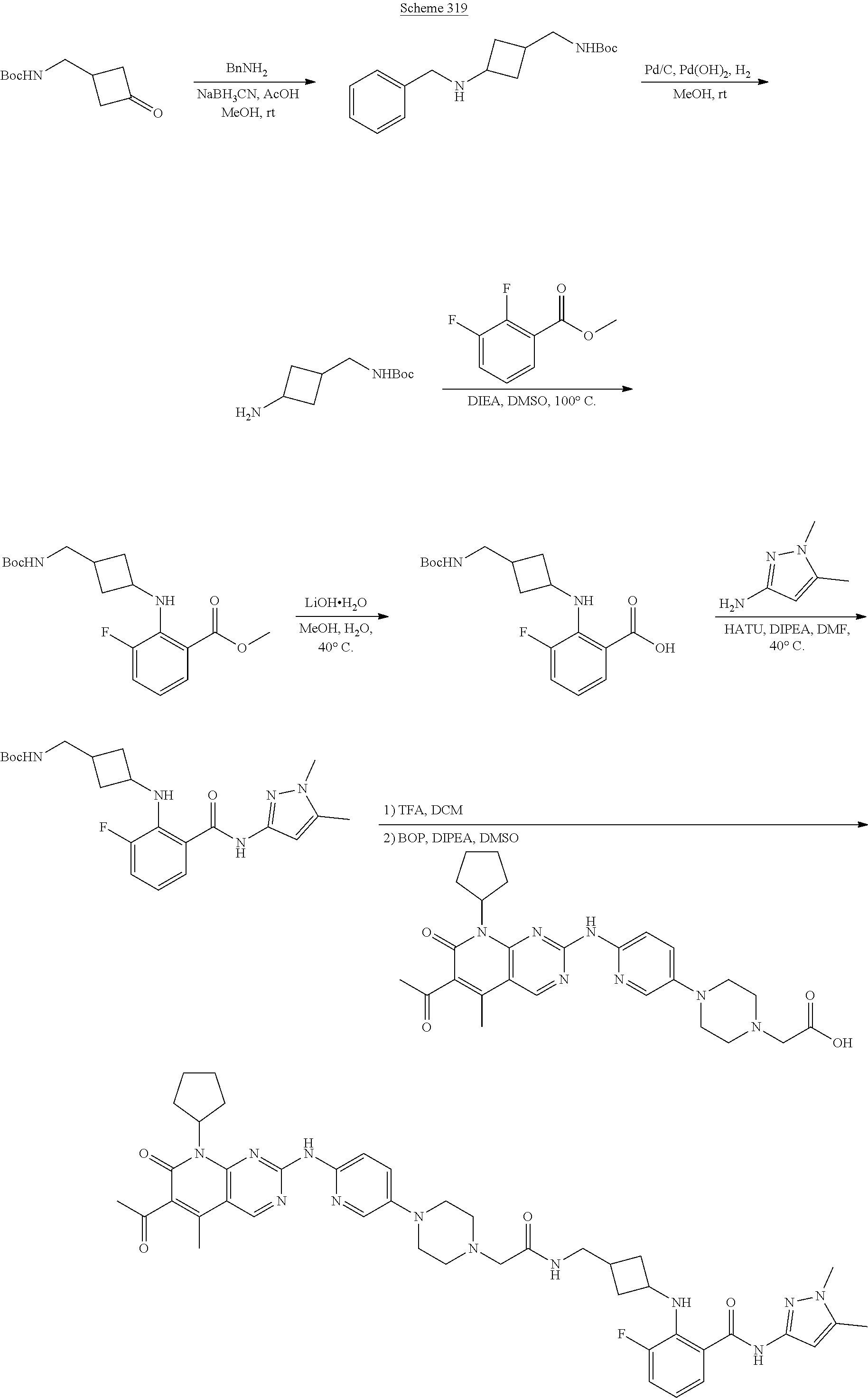

### Step 1. Synthesis of tert-butyl ((3-(benzylamino)cyclobutyl)methyl)carbamate

To a solution of tert-butyl ((3-oxocyclobutyl)methyl)carbamate (2.00 g, 10.0 mmol) and BnNH₂ (2.69 g, 25.0 mmol) in MeOH (20 mL) were added NaBH₃CN (1.01 g, 16.0 mmol) and AcOH (1.02 g, 17.0 mmol). The mixture was stirred at rt for 12 h. The mixture was concentrated under reduced pressure. The residue was diluted with saturated aq. NH₄Cl solution (50 mL) and extracted with ethyl acetate (50 mL×3). The combined organic layers were washed with brine (100 mL), dried over Na₂SO₄, filtered and concentrated. The crude product was purified by pre-HPLC to provide the desired product (2.00 g, 66% yield) as a white solid. ¹HNMR (400 MHz, DMSO) δ 8.30 (s, 1H), 7.40-7.22 (m, 5H), 6.90-6.75 (m, 1H), 3.73 (s, 2H), 3.38-3.10 (m, 1H), 2.99-2.87 (m, 2H), 2.30-2.08 (m, 2H), 2.02-1.84 (m, 2H), 1.57-1.51 (m, 1H), 1.37 (s, 9H).

### Step 2. Synthesis of tert-butyl ((3-aminocyclobutyl)methyl)carbamate

To a solution of tert-butyl N-[[3-(benzylamino)cyclobutyl]methyl]carbamate (1.59 g, 5.49 mmol) in MeOH (20 mL) were added Pd(OH)₂ (615 mg) and Pd/C (580 mg) under N₂ atmosphere. The suspension was degassed and purged with H₂ for 3 times. The mixture was stirred under H₂ (15 psi) at rt for 12 h. The reaction mixture was filtered through a pad of Celite to provide the desired product (600 mg, 55% yield) as a white solid. ¹HNMR (400 MHz, DMSO-d₆) δ 5.07-4.34 (m, 3H), 3.26-3.11 (m, 1H), 3.02-2.94 (m, 1H), 2.89 (t, J=5.6 Hz, 1H), 2.22-2.09 (m, 2H), 1.94-1.78 (m, 2H), 1.46-1.42 (m, 1H), 1.37 (s, 9H).

The remaining steps were performed following the standard procedures for preparing CPD-261 to provide the desired product (15.0 mg, 6% yield over 5 steps) as a yellow solid. MS (ESI) m/z=819.6 [M+H]⁺.

### Example 320. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(1,5-dimethyl-1H-pyrazol-3-yl)-3-(trifluoromethyl)benzamide (CPD-266)

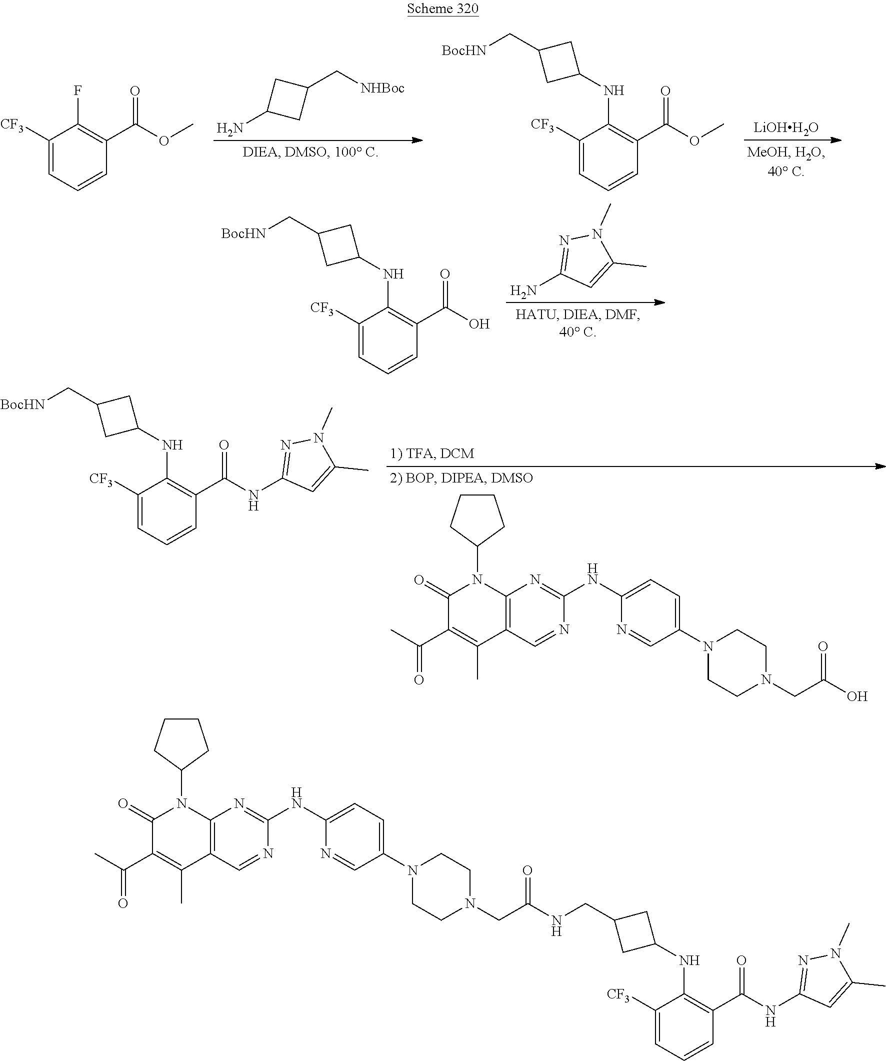

CPD-266 was synthesized following the standard procedures for preparing CPD-261 (15.0 mg, 4% yield over 5 steps) as a yellow solid. MS (ESI) m/z=869.7 [M+H]⁺.

### Example 321. 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-(methylamino)nicotinamide (CPD-267)

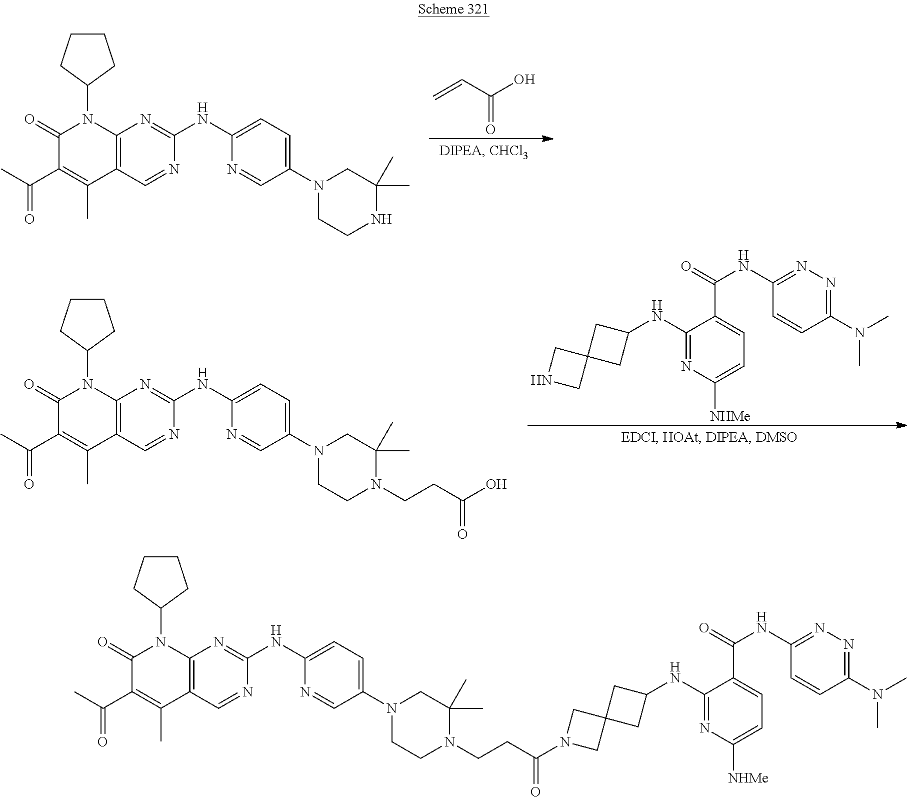

### Step 1. Synthesis of 3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoic acid

To a mixture of 6-acetyl-8-cyclopentyl-2-((5-(3,3-dimethylpiperazin-1-yl)pyridin-2-yl)amino)-5-methylpyrido[2,3-d]pyrimidin-7(8H)-one (9 mg, 18.92 μmol) and acrylic acid (13.6 mg, 189.2 μmol) in CHCl₃ (5 mL) was added DIPEA (24.5 mg, 189.2 μmol). The mixture was stirred at 90° C. for 16 h, before it was concentrated and purified by reverse-phase chromatography to give the desired product MS (ESI) m/z=548.5 [M+H]⁺.

### Step 2. Synthesis of 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-(methylamino)nicotinamide

To a mixture of 3-[4-[6-[(6-acetyl-8-cyclopentyl-5-methyl-7-oxo-pyrido[2,3-d]pyrimidin-2-yl)amino]-3-pyridyl]-2,2-dimethyl-piperazin-1-yl]propanoic acid (7 mg, 12.8 μmol), DIPEA (16.5 mg, 127.8 mol) and HOAt (3.48 mg, 25.6 μmol) in DMSO (1 mL) were added EDCI (4.90 mg, 25.6 μmol) and 2-(2-azaspiro[3.3]heptan-6-ylamino)-N-[6-(dimethylamino)pyridazin-3-yl]-6-(methylamino)pyridine-3-carboxamide (4.9 mg, 12.8 μmol). The mixture was stirred at rt for 10 h, before it was purified by prep-HPLC to provide the title compound (3 mg, 26% yield) as a white solid. MS (ESI) m/z=912.6 [M+H]⁺.

### Example 322. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.3]heptan-6-yl)amino)-N-(6-(dimethylamino)pyridazin-3-yl)-6-(methylamino)nicotinamide (CPD-268)

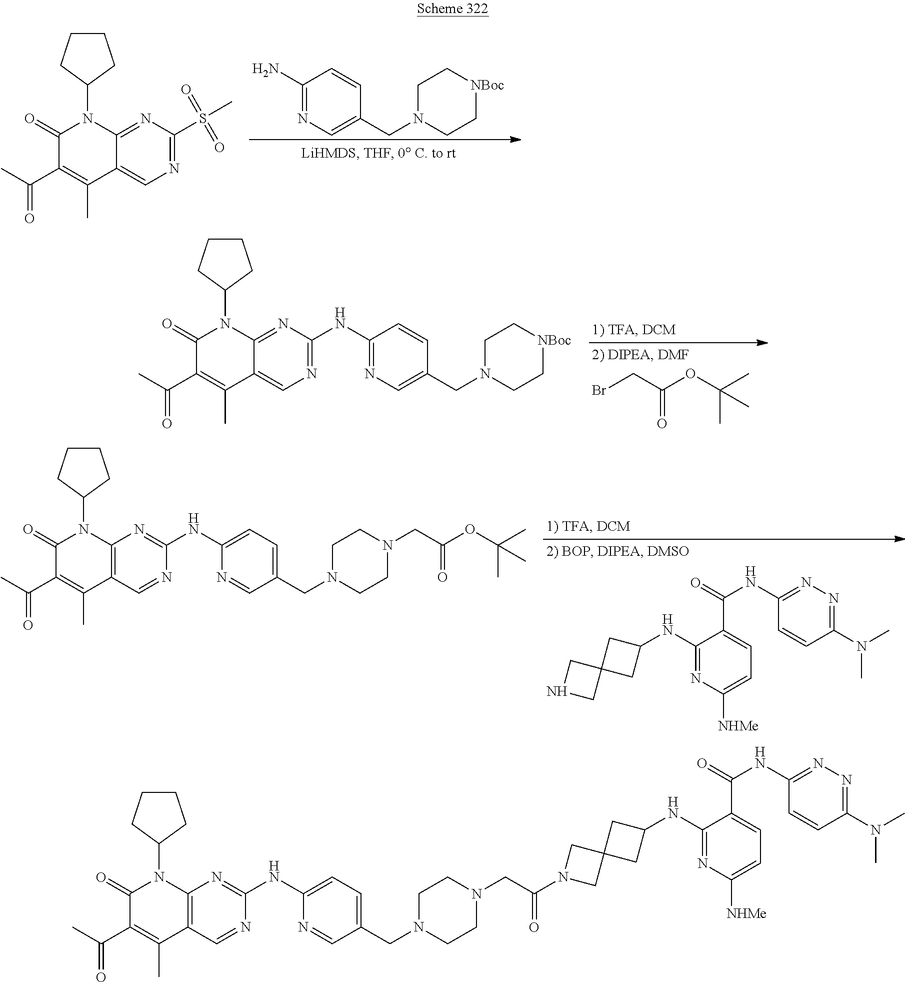

### Step 1. Synthesis of tert-butyl 4-((6-((6-acetyl-8-cyclopentyl-5-methyl-7-oxo-7,8-dihydropyrido[2,3-d]pyrimidin-2-yl)amino)pyridin-3-yl)methyl)piperazine-1-carboxylate

To a solution of tert-butyl (1-(6-aminopyridin-3-yl)piperidin-4-yl)carbamate (177 mg, 0.94 mmol) and 6-acetyl-8-cyclopentyl-5-methyl-2-(methylsulfonyl)pyrido[2,3-d]pyrimidin-7(8H)-one (164 mg, 10.98 mmol) in THF (5 ml) was added LiHMDS (1 M, 1.88 mL, 1.88 mmol) slowly at 0° C. The mixture was stirred at rt for 2 h. The mixture was quenched with water (20 mL) and extracted with ethyl acetate (30 mL×3). The combined organic phase was washed with brine, dried over Na₂SO₄, filtered and concentrated. The residue was purified by reverse-phase chromatography to afford the desired compound as a yellow solid (92 mg, 17% yield). MS (ESI) m/z=562.4 [M+H]⁺.

### Step 2 and step 3. Synthesis of tert-butyl 2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetate

To a solution of tert-butyl 4-((6-((6-acetyl-8-cyclopentyl-5-methyl-7-oxo-7,8-dihydropyrido[2,3-d]pyrimidin-2-yl)amino)pyridin-3-yl)methyl)piperazine-1-carboxylate (92 mg, 0.16 mmol) in dichloromethane (3 ml) was added TFA (0.3 ml). The mixture was stirred at rt for 4 h. The mixture was concentrated to afford the desired compound (123.9 mg, crude) as a brown oil, which was used in the next step directly MS (ESI) m/z=462.3 [M+H]⁺.

To a solution of 6-acetyl-8-cyclopentyl-5-methyl-2-((5-(piperazin-1-ylmethyl)pyridin-2-yl)amino)pyrido[2,3-d]pyrimidin-7(8H)-one (80 mg, 0.17 mmol) in DMF (5 mL) were added tert-butyl 2-bromoacetate (67.62 mg, 0.35 mmol) and DIPEA (112 mg, 0.87 mmol). The mixture was stirred at rt for 2 h. The mixture was poured into water (30 mL) and extracted with ethyl acetate (3×50 mL). The combined organic phase was washed with brine, dried over anhydrous Na₂SO₄, filtered and concentrated. The residue was purified by silica gel chromatography to provide the desired product (80 mg, 80% yield) as a light-yellow solid. MS(ESI) m/z=576.5 [M+H]⁺.

The remaining steps were performed following the standard procedures for preparing CPD-261 to provide the desired product (9 mg, 32% yield over 2 steps) as a yellow solid. MS (ESI) m/z=884.6 [M+H]⁺.

### Example 323. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)azetidin-3-yl)amino)-N-(6-(dimethylamino)pyridazin-3-yl)-6-(methylamino)nicotinamide (CPD-269)

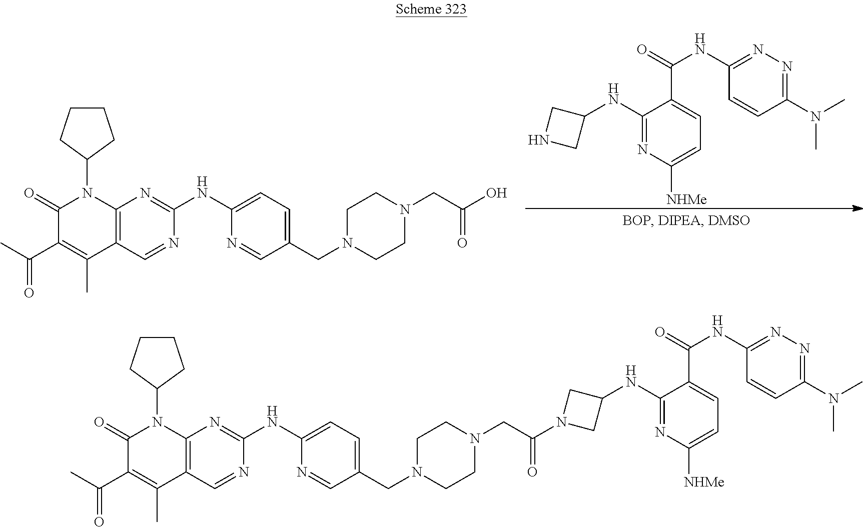 [SMILES: CC(=O)c1c(C)c2cnc(Nc3ccc(CN4CCN(CC(=O)O)CC4)cn3)nc2n(C2CCCC2)c1=O ; CNc1ccc(C(=O)Nc2ccc(N(C)C)nn2)c(NC2CNC2)n1 ; CNc1ccc(C(=O)Nc2ccc(N(C)C)nn2)c(NC2CN(C(=O)CN3CCN(Cc4ccc(Nc5ncc6c(C)c(C(C)=O)c(=O)n(C7CCCC7)c6n5)nc4)CC3)C2)n1]

CPD-269 was synthesized following the standard procedure for preparing CPD-261 (10.0 mg, 35% yield) as a yellow solid. MS (ESI) m/z=844.7 [M+H]⁺.

### Example 324. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-((methyl-d₃)amino)-N-(6-methylpyridin-2-yl)nicotinamide (CPD-270)

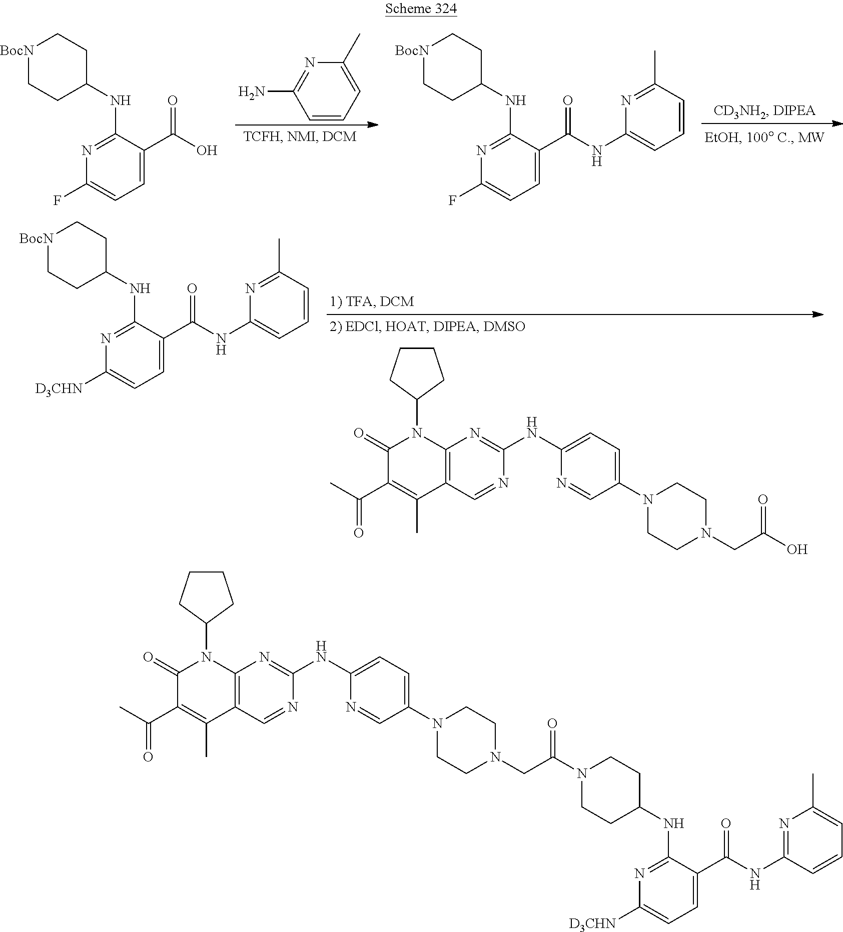

CPD-270 was synthesized following the standard procedures for preparing CPD-144 (17.0 mg, 8% yield over 4 steps) as a yellow solid. MS (ESI) m/z=831.8 [M+H]⁺.

### Example 325. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclopentyl)amino)-N-(6-(dimethylamino)pyridazin-3-yl)-6-(methylamino)nicotinamide (CPD-271)

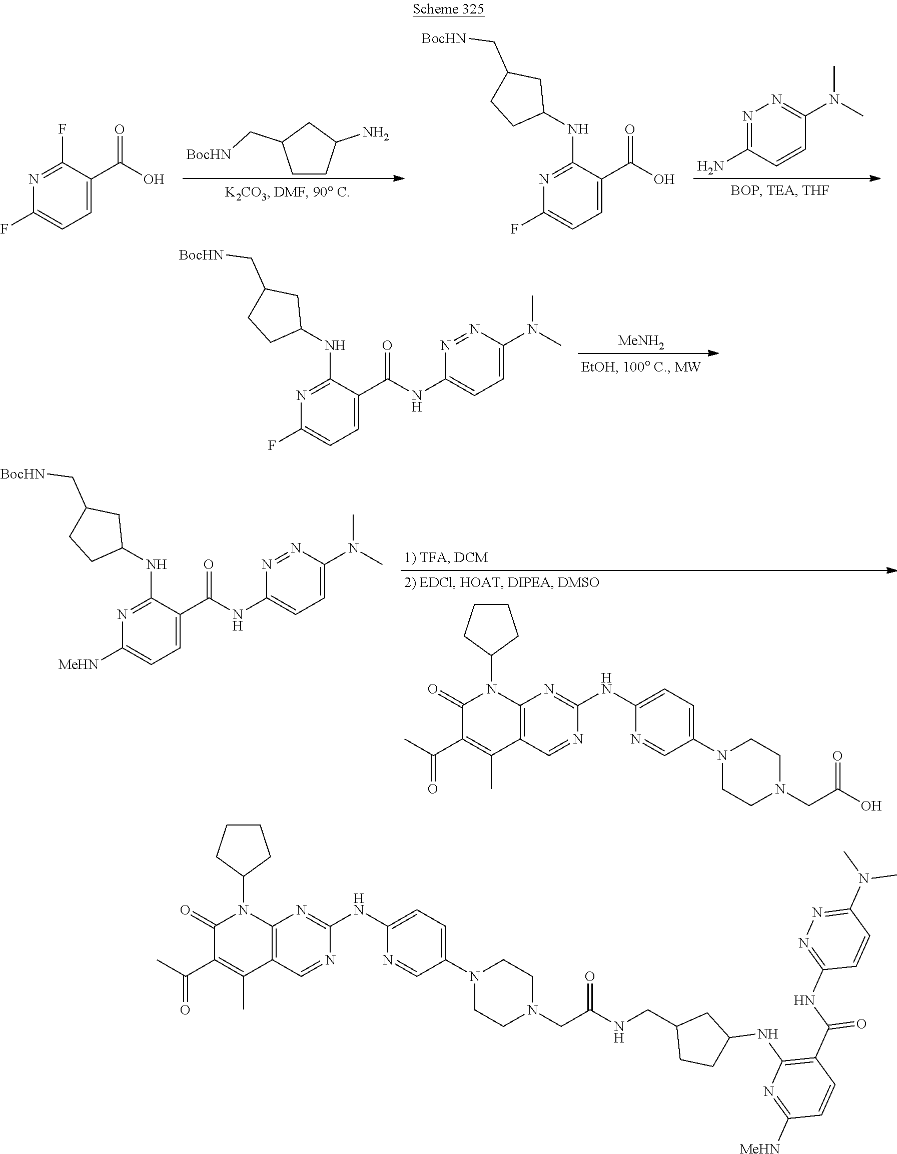

CPD-271 was synthesized following the standard procedures for preparing CPD-145 (2.1 mg, 1% yield over 5 steps) as a yellow solid. MS (ESI) m/z=872.7 [M+H]⁺.

### Example 326. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6-(dimethylamino)pyridazin-3-yl)-6-(isopropylamino)nicotinamide (CPD-272)

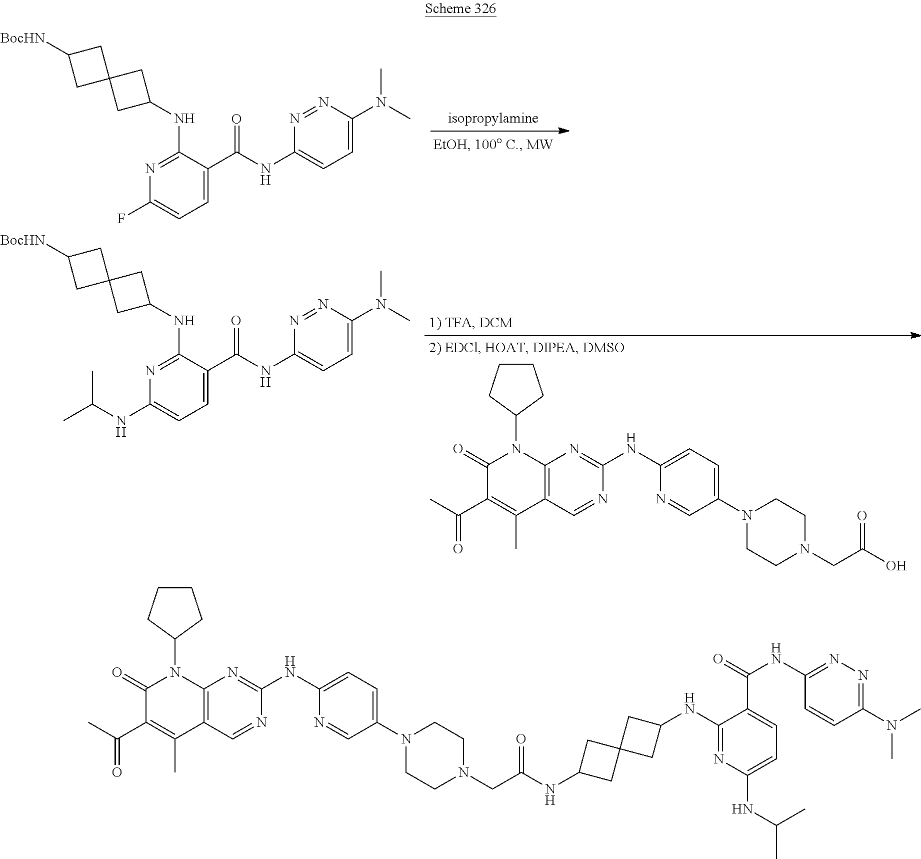

CPD-272 was synthesized following the standard procedures for preparing CPD-144 (2.2 mg, 4% yield over 3 steps) as a yellow solid. MS (ESI) m/z=912.7 [M+H]⁺.

### Example 327. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(2-cyclopropyl-1-methyl-1H-imidazol-4-yl)-6-((methyl-d₃)amino)nicotinamide (CPD-273)

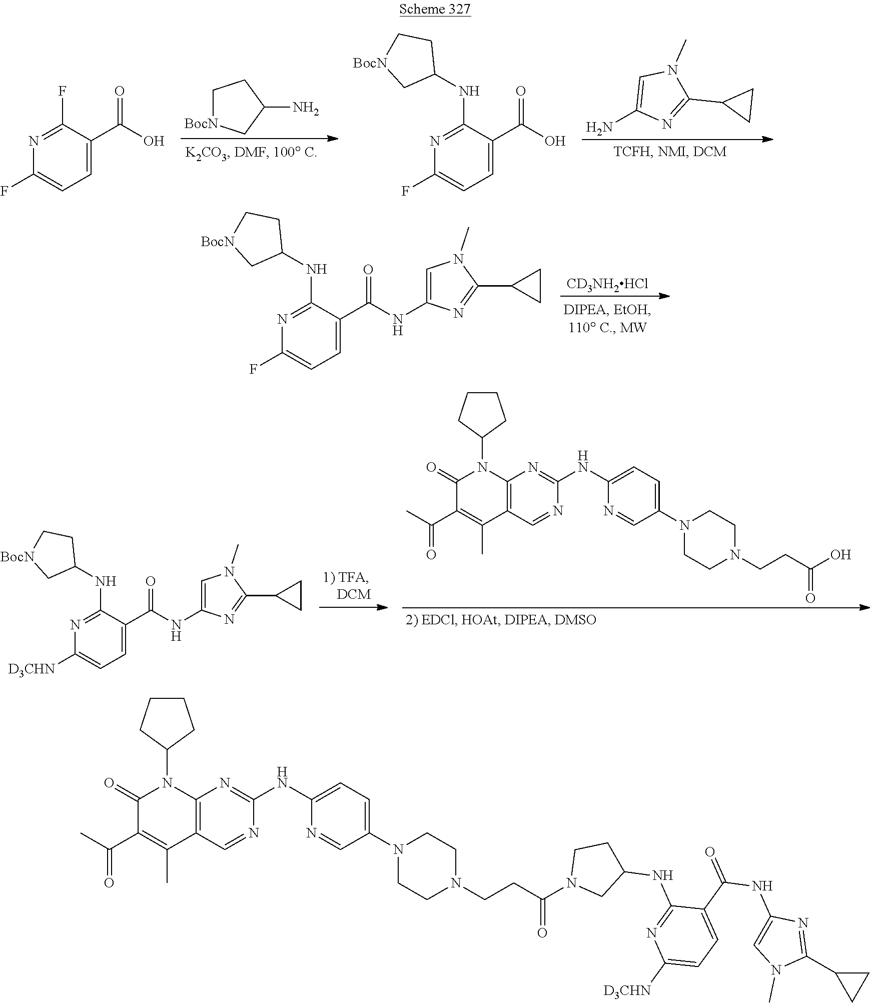

### Step 1. Synthesis of 2-((1-(tert-butoxycarbonyl)pyrrolidin-3-yl)amino)-6-fluoronicotinic acid

A mixture of tert-butyl 3-aminopyrrolidine-1-carboxylate (293 mg, 1.57 mmol), 2,6-difluoropyridine-3-carboxylic acid (500 mg, 3.14 mmol) and K₂CO₃ (652 mg, 4.71 mmol) in DMF (4 mL) was stirred at 100° C. for 12 h. The mixture was cooled to rt. The mixture was diluted with water (30 mL) and extracted with dichloromethane (3×15 mL). The combined organic phase was washed with brine, dried over Na₂SO₄, filtered, and concentrated to give the crude. The residue was purified by reverse phase chromatography to provide the desired product (350 mg, 69% yield) as a gray solid. MS (ESI) m/z=326.2 [M+H]⁺.

### Step 2. Synthesis of tert-butyl 3-((3-((2-cyclopropyl-1-methyl-1H-imidazol-4-yl) carbamoyl)-6-fluoropyridin-2-yl)amino)pyrrolidine-1-carboxylate

To a mixture of 2-[(1-tert-butoxycarbonylpyrrolidin-3-yl)amino]-6-fluoronicotinic acid (80 mg, 0.25 mmol) and NMI (101 mg, 1.23 mmol) in dichloromethane (5 mL) were added 2-cyclopropyl-1-methyl-imidazol-4-amine (40.5 mg, 0.29 mmol) and TCFH (207 mg, 0.74 mmol). The mixture was stirred at rt for 2 h. The mixture was quenched with water (5 mL) and extracted with dichloromethane (3×10 mL). The combined organic phase was washed with brine, dried over Na₂SO₄, filtered, and concentrated to give the crude. The residue was purified by silica gel chromatography to provide the desired product (40 mg, 37% yield) as a white solid. MS (ESI) m/z=445.8 [M+H]⁺.

### Step 3. Synthesis of tert-butyl 3-((3-((2-cyclopropyl-1-methyl-1H-imidazol-4-yl) carbamoyl)-6-((methyl-d₃)amino)pyridin-2-yl)amino)pyrrolidine-1-carboxylate

To a solution of tert-butyl 3-((3-((2-cyclopropyl-1-methyl-1H-imidazol-4-yl)carbamoyl)-6-fluoropyridin-2-yl)amino)pyrrolidine-1-carboxylate (20 mg, 45 μmol) and DIPEA (87.2 mg, 675 μmol) in EtOH (1 mL) was added CD₃NH₂·HCl (31.7 mg, 450 μmol) at rt. The reaction mixture was stirred at 110° C. under N₂ for 3 h under microwave irradiation. Upon completion, the mixture was cooled to rt and concentrated to give the crude. The residue was purified by reverse-phase chromatography to provide the desired product (5 mg, 24% yield) as a white solid. MS (ESI) m/z=459.6 [M+H]⁺.

### Step 4. Synthesis of N-(2-cyclopropyl-1-methyl-1H-imidazol-4-yl)-6-((methyl-d₃)amino)-2-(pyrrolidin-3-ylamino)nicotinamide

A solution of tert-butyl 3-((3-((2-cyclopropyl-1-methyl-1H-imidazol-4-yl)carbamoyl)-6-((methyl-d₃)amino)pyridin-2-yl)amino)pyrrolidine-1-carboxylate (5 mg, 11 μmol) in dichloromethane (1 mL) and TFA (0.5 mL) was stirred at rt for 3 h. The reaction mixture was concentrated to provide the desired product (3.5 mg, 90% yield) as a brown oil. This compound was used directly in the next step without further purification. MS (ESI) m/z=359.5 [M+H]⁺.

### Step 5. Synthesis of 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrolidin-3-yl)amino)-N-(2-cyclopropyl-1-methyl-1H-imidazol-4-yl)-6-((methyl-d3)amino) nicotinamide

To a mixture of N-(2-cyclopropyl-1-methyl-1H-imidazol-4-yl)-6-((methyl-d₃)amino)-2-(pyrrolidin-3-ylamino)nicotinamide (3.5 mg, 9.8 μmol), EDCI (4.7 mg, 24.4 μmol) and HOAt (1.3 mg, 9.8 μmol) in DMSO (1 mL) were added DIPEA (5.1 mg, 39.1 μmol) and 3-[4-[6-[(6-acetyl-8-cyclopentyl-5-methyl-7-oxo-pyrido[2,3-d]pyrimidin-2-yl)amino]-3-pyridyl]piperazin-1-yl]propanoic acid (5.1 mg, 9.8 μmol). The mixture was stirred at rt for 12 h. The mixture was purified by prep-HPLC to provide the desired product (1.67 mg, 20% yield) as a yellow solid. MS (ESI) m/z=860.6 [M+H]⁺.

### Example 328. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)pyrrolidin-3-yl)amino)-N-(2-cyclopropyl-1-methyl-1H-imidazol-4-yl)-6-(isopropylamino)nicotinamide (CPD-274)

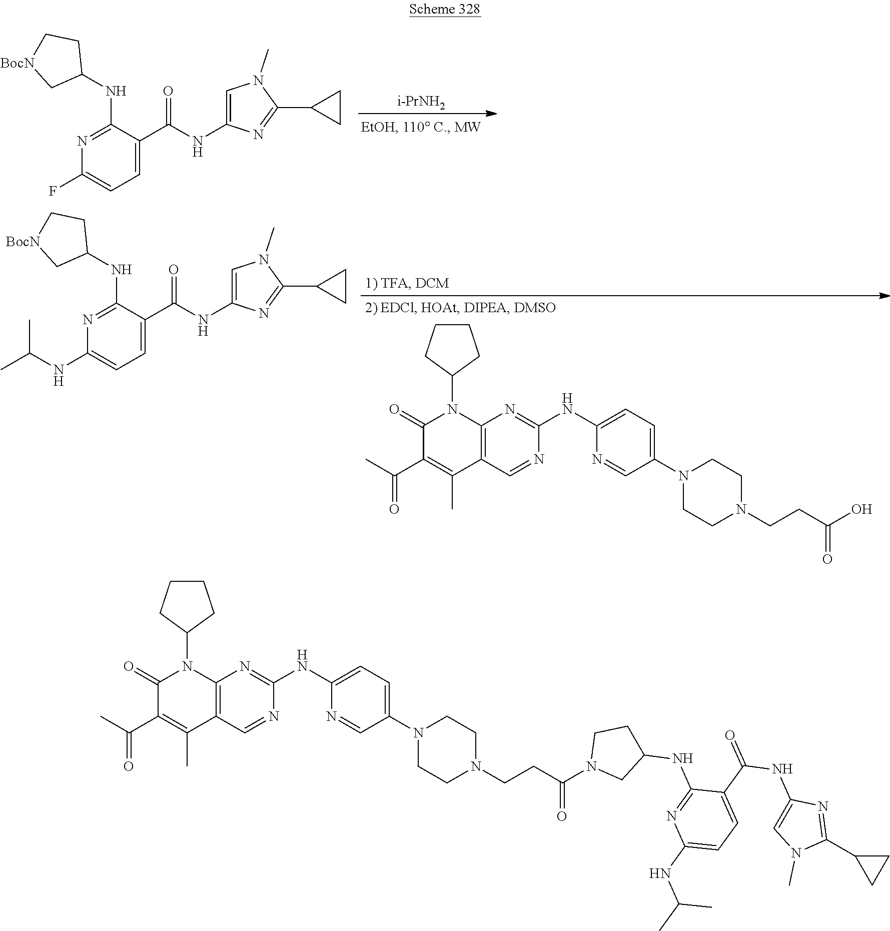

CPD-274 was synthesized following the standard procedures for preparing CPD-144 (1.81 mg, 5% yield for 3 steps) as a yellow solid. MS (ESI) m/z=885.6 [M+H]⁺.

### Example 329. 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(2-cyclopropyl-1-methyl-1H-imidazol-4-yl)-6-(isopropylamino)nicotinamide (CPD-275)

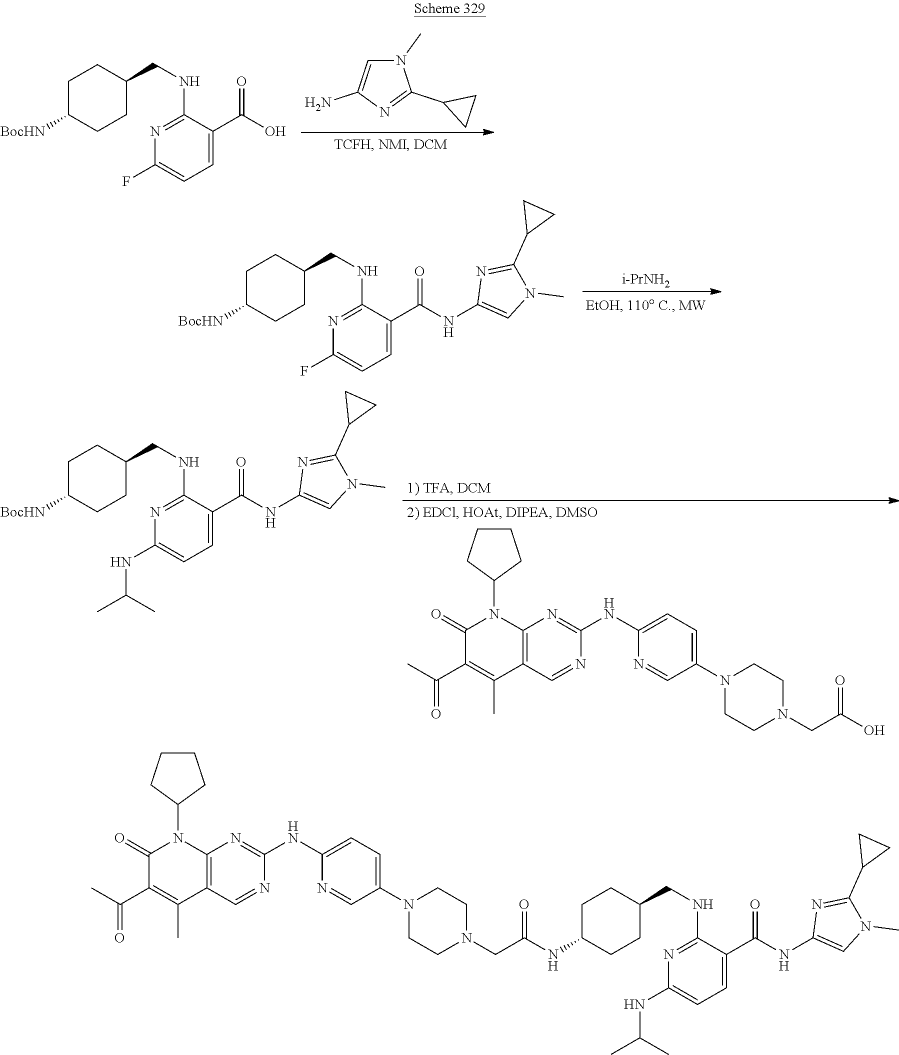

CPD-275 was synthesized following the standard procedures for preparing CPD-144 (5.1 mg, 3% yield for 4 steps) as a yellow solid. MS (ESI) m/z=913.8 [M+H]⁺.

### Example 330. 2-(((trans-4-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclohexyl)methyl)amino)-N-(2-cyclopropyl-1-methyl-1H-imidazol-4-yl)-6-((methyl-d₃)amino)nicotinamide (CPD-276)

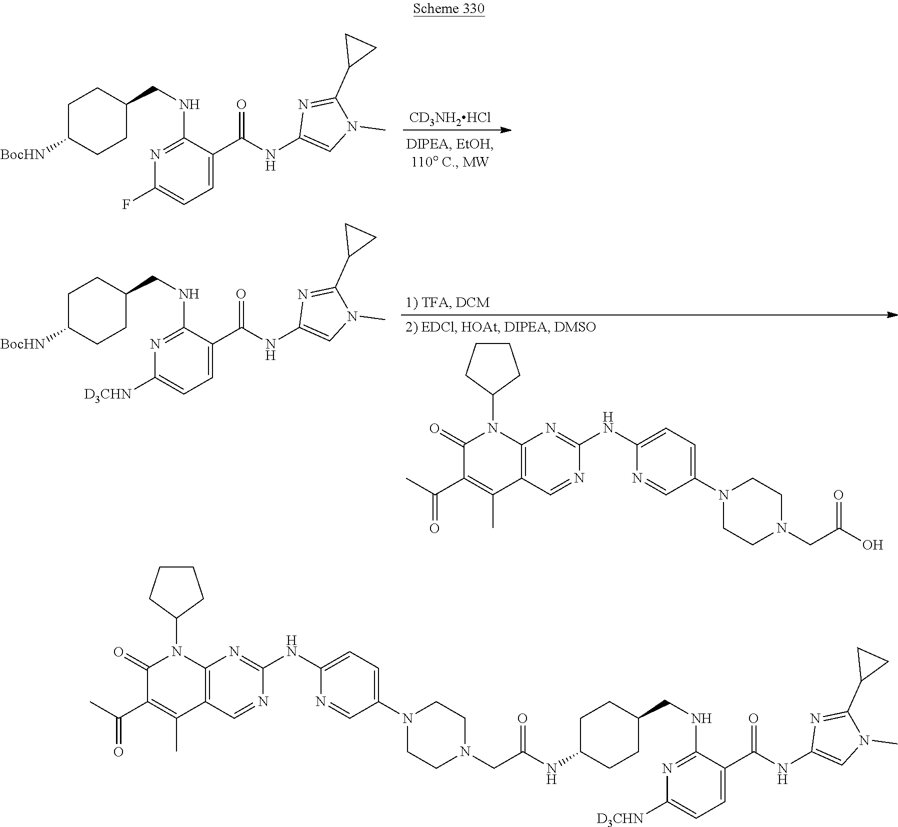

CPD-276 was synthesized following the standard procedures for preparing CPD-144 (4.3 mg, 16% yield for 3 steps) as a yellow solid. MS (ESI) m/z 888.7 [M+H]⁺.

### Example 331. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-(isopropylamino)nicotinamide (CPD-277)

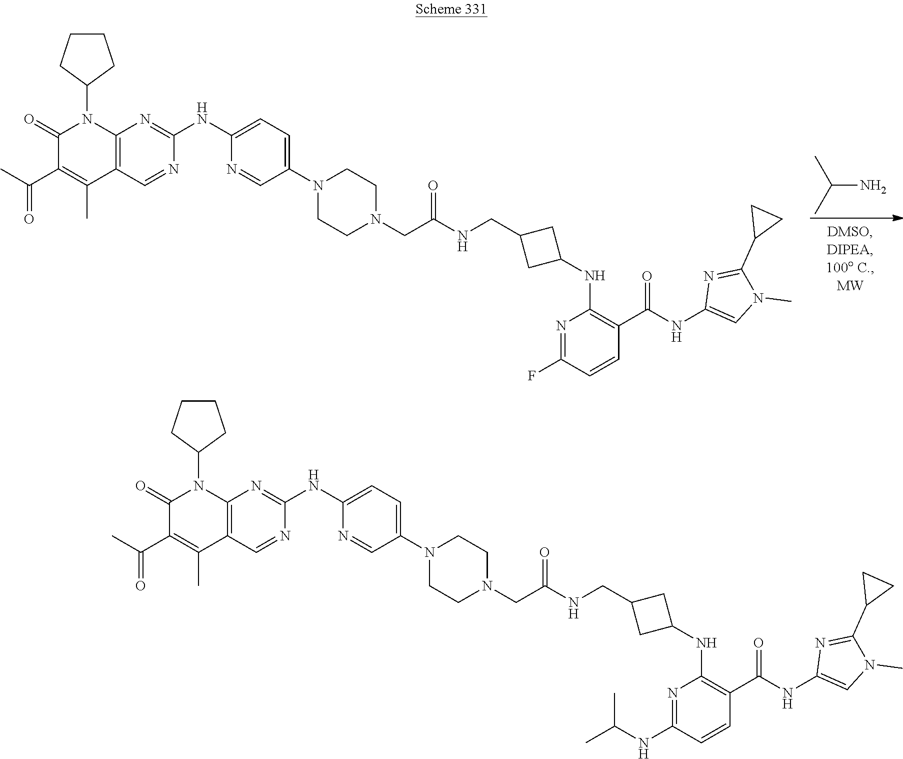

CPD-277 was synthesized following the standard procedure for preparing CPD-253 (12.1 mg, 85% yield) as a yellow solid. MS (ESI) m/z=885.7 [M+H]⁺.

### Example 332. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(2-cyclopropyl-1-methyl-1H-imidazol-4-yl)-6-((methyl-d₃)amino)nicotinamide (CPD-278)

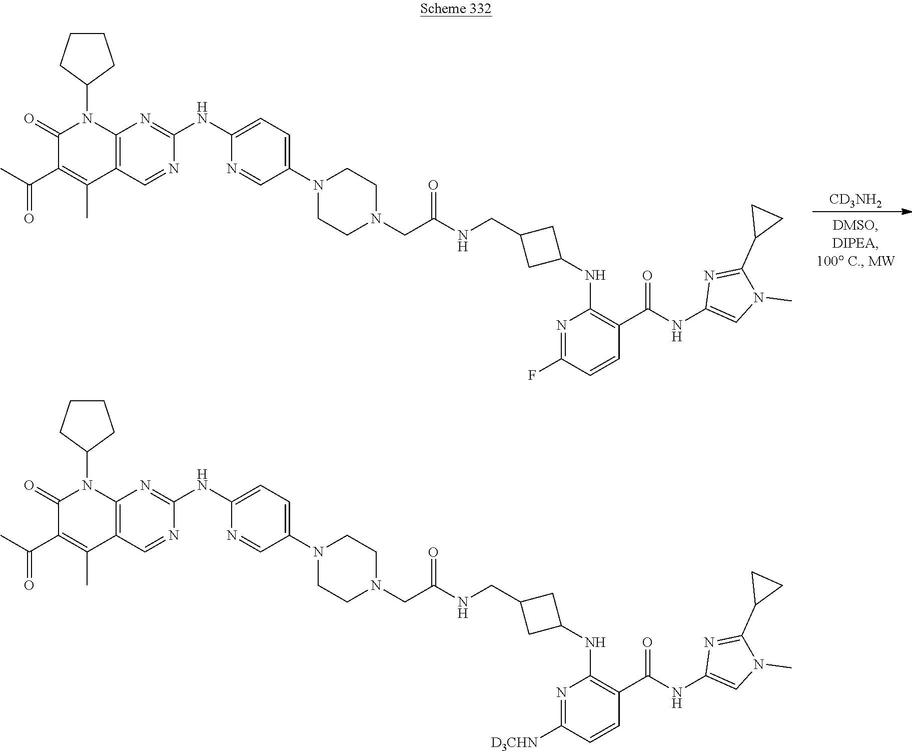

CPD-278 was synthesized following the standard procedure for preparing CPD-253 (12.4 mg, 89% yield) as a yellow solid. MS (ESI) m/z=860.6 [M+H]⁺.

### Example 333. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(2-cyclopropyl-1-methyl-1H-imidazol-4-yl)-6-(isopropylamino)nicotinamide (CPD-279)

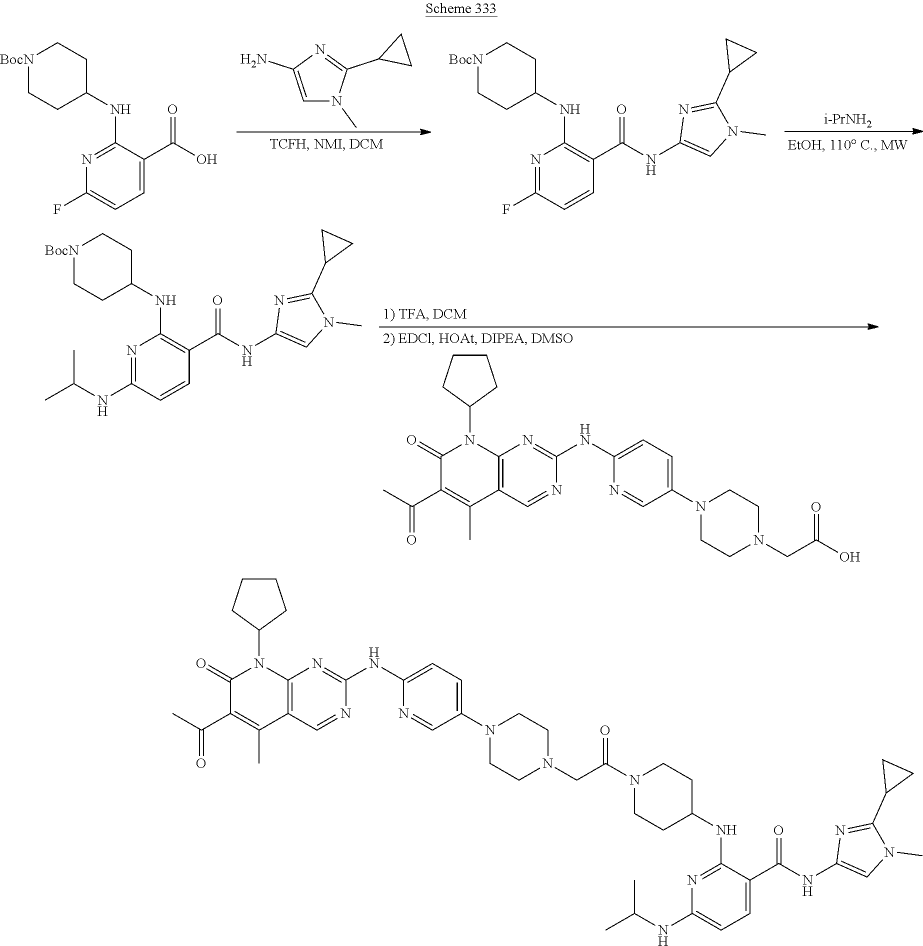

CPD-279 was synthesized following the standard procedures for preparing CPD-144 (13.5 mg, 21% yield for 4 steps) as a yellow solid. MS (ESI) m/z=885.7 [M+H]⁺.

### Example 334. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(6-(dimethylamino)pyridazin-3-yl)-6-(isopropylamino)nicotinamide (CPD-280)

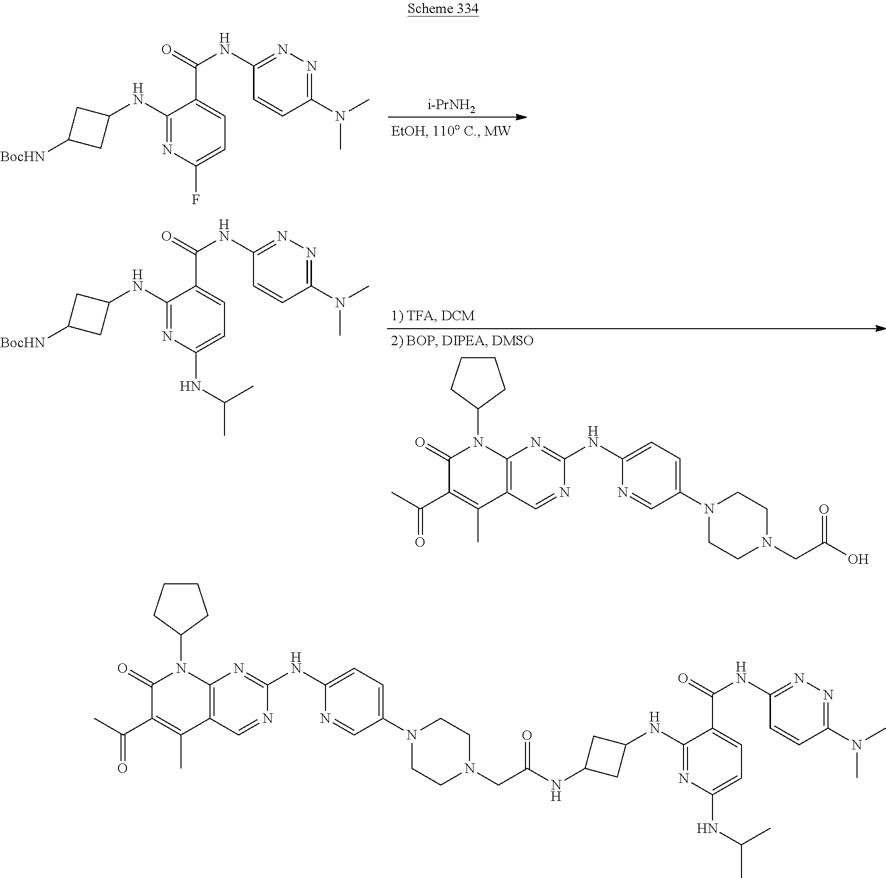

CPD-280 was synthesized following the standard procedures for preparing CPD-144 (10.0 mg, 10% yield for 3 steps) as a yellow solid. MS (ESI) m/z=872.6 [M+H]⁺.

### Example 335. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(methylamino)-N-(5-methylisoxazol-3-yl)nicotinamide (CPD-281)

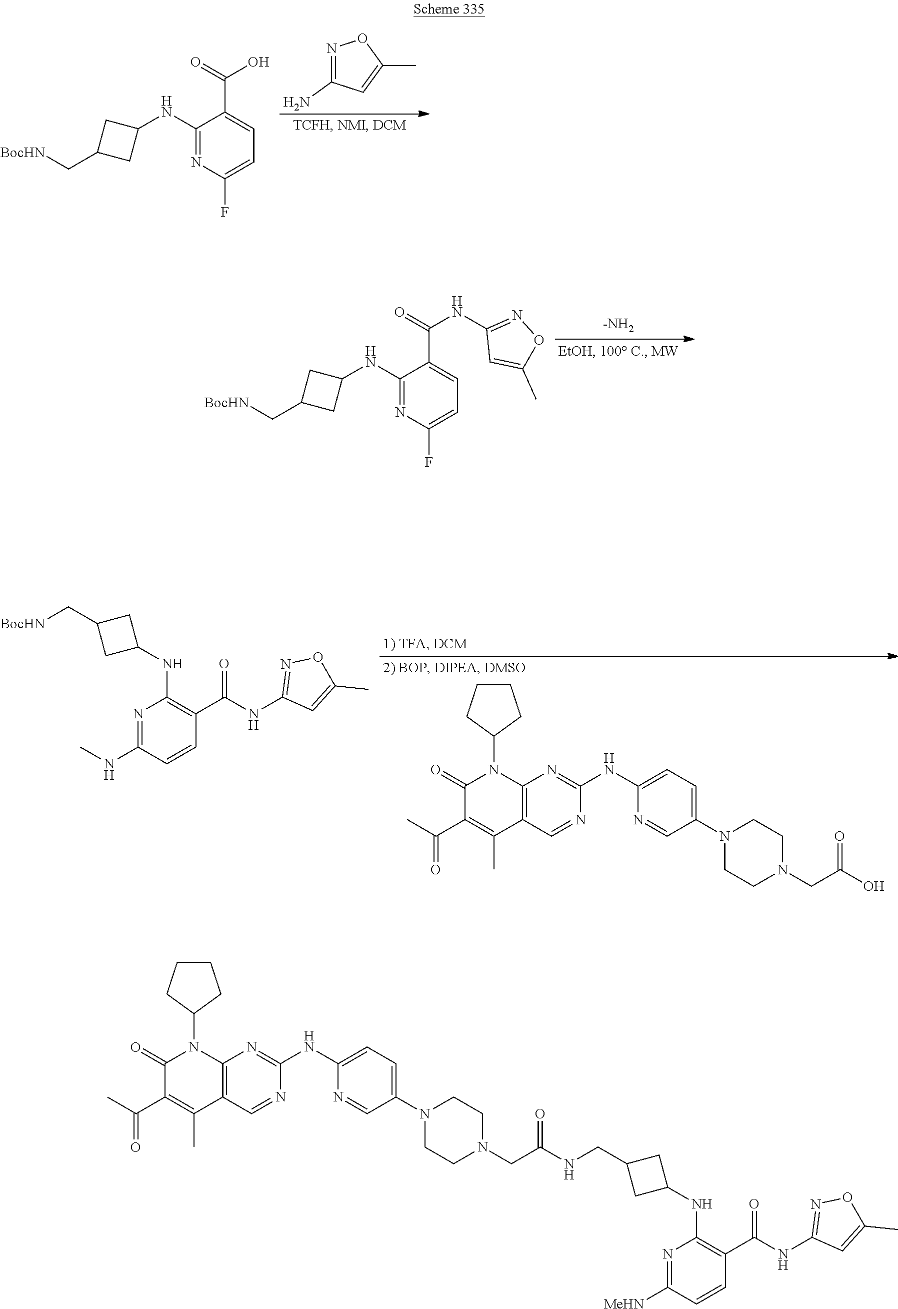

CPD-281 was synthesized following the standard procedures for preparing CPD-145 (5.0 mg, 5% yield for 6 steps) as a yellow solid. MS (ESI) m/z=818.7 [M+H]⁺.

### Example 336. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-(isopropylamino)-N-(5-methylisoxazol-3-yl)nicotinamide (CPD-282)

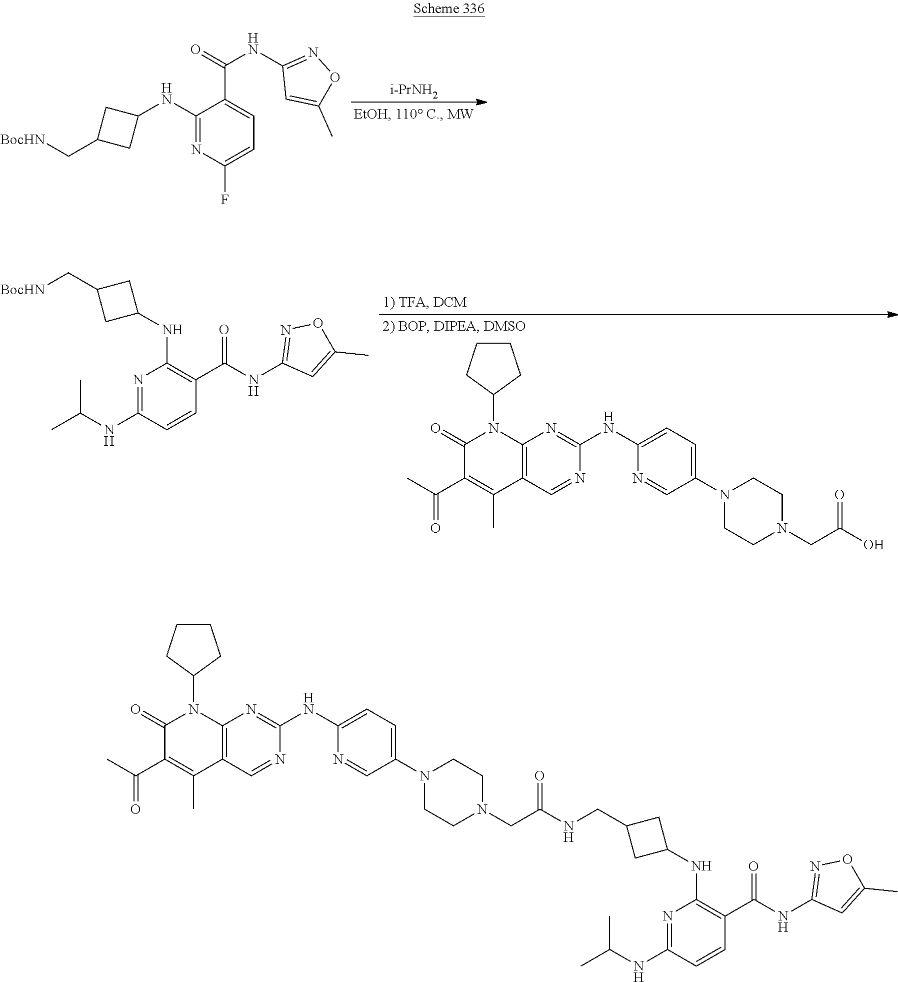 [SMILES: "i-PrNH2" ; Cc1cc(NC(=O)c2ccc(F)nc2NC2CC(CNC(=O)OC(C)(C)C)C2)no1 ; Cc1cc(NC(=O)c2ccc(NC(C)C)nc2NC2CC(CNC(=O)OC(C)(C)C)C2)no1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCC5CC(Nc6nc(NC(C)C)ccc6C(=O)Nc6cc(C)on6)C5)CC4)cn3)nc2n(C2CCCC2)c1=O]

CPD-282 was synthesized following the standard procedures for preparing CPD-144 (5.0 mg, 10% yield for 3 steps) as a yellow solid. MS (ESI) m/z=846.7 [M+H]⁺.

### Example 337. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-6-((methyl-d₃)amino)-N-(5-methylisoxazol-3-yl)nicotinamide (CPD-283)

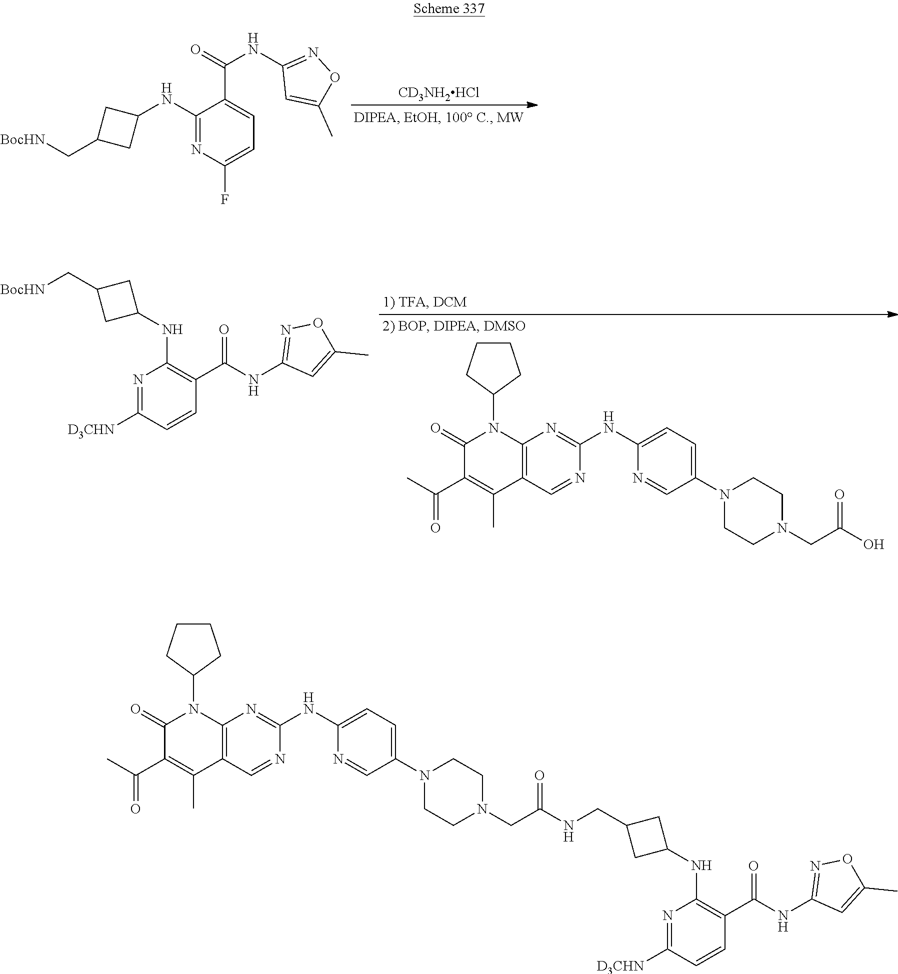

CPD-283 was synthesized following the standard procedures for preparing CPD-144 (8.0 mg, 16% yield for 3 steps) as a yellow solid. MS (ESI) m/z=821.7 [M+H]⁺.

### Example 338. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(2-cyclopropyl-1-methyl-1H-imidazol-4-yl)-6-(isopropylamino)nicotinamide (CPD-284)

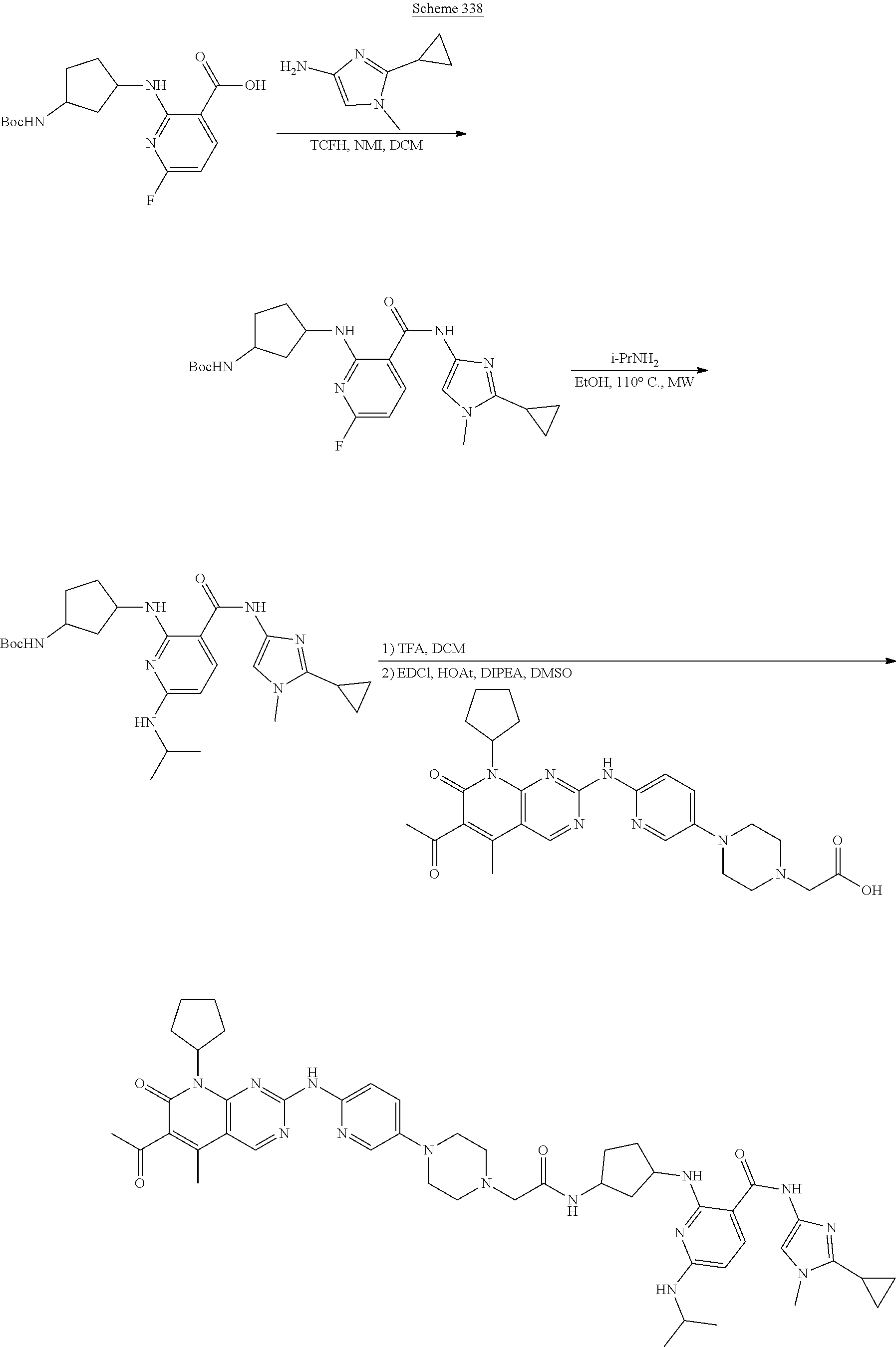

CPD-284 was synthesized following the standard procedures for preparing CPD-144 (3.0 mg, 6% yield for 4 steps) as a yellow solid. MS (ESI) m/z=885.4 [M+H]⁺.

### Example 339. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-N-(2-cyclopropyl-1-methyl-1H-imidazol-4-yl)-6-((methyl-d₃)amino)nicotinamide (CPD-285)

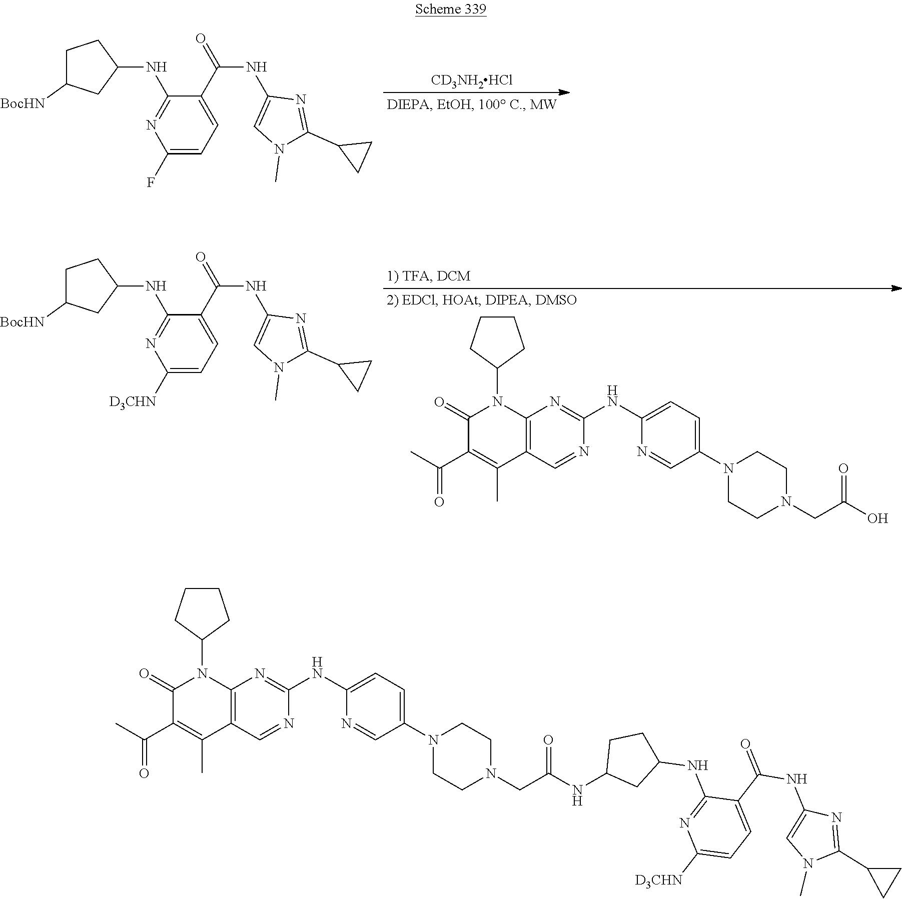

CPD-285 was synthesized following the standard procedures for preparing CPD-144 (3.5 mg, 10% yield for 3 steps) as a yellow solid. MS (ESI) m/z=860.7 [M+H]⁺.

### Example 340. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-4-methyl-N-(5-methylisoxazol-3-yl)benzamide (CPD-286)

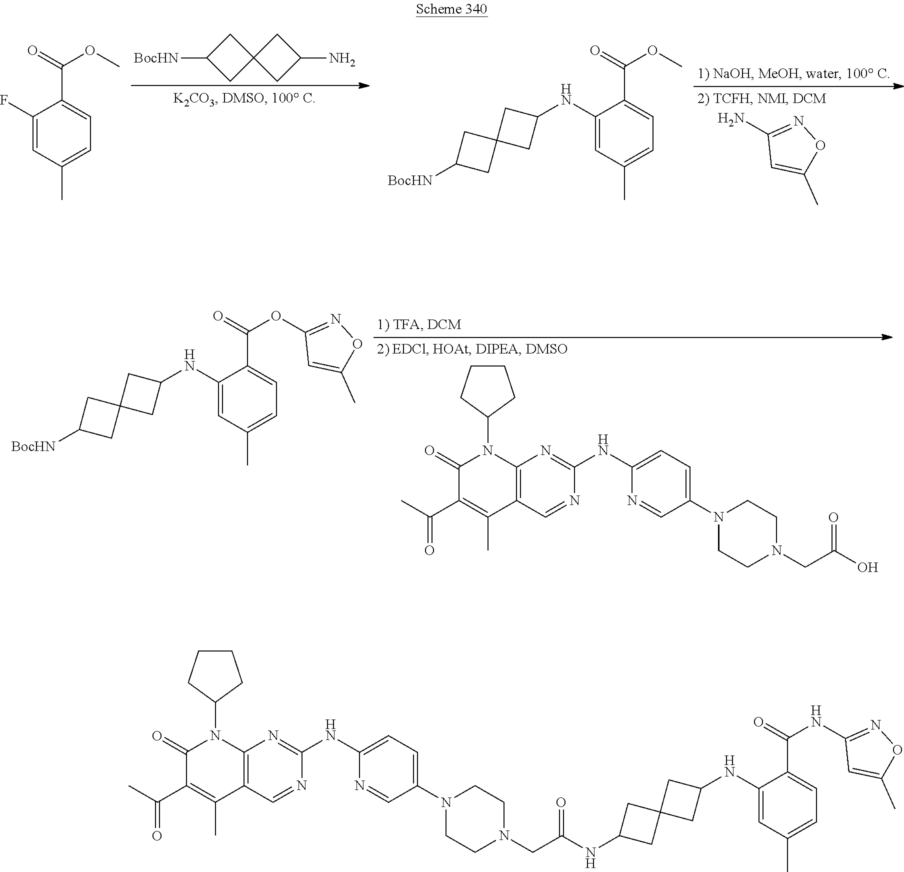

CPD-286 was synthesized following the standard procedures for preparing CPD-255 (0.8 mg, 1% yield over 5 steps) as a yellow solid. MS (ESI) m/z=828.7 [M+H]⁺.

### Example 341. 4-(6-((6-acetyl-8-cyclopentyl-5-methyl-7-oxo-7,8-dihydropyrido[2,3-d]pyrimidin-2-yl)amino)pyridin-3-yl)-N-(3-((3-((2-cyclopropyl-1-methyl-1H-imidazol-4-yl)carbamoyl)-6-((methyl-d3)amino)pyridin-2-yl)amino)cyclobutyl)piperazine-1-carboxamide (CPD-287)

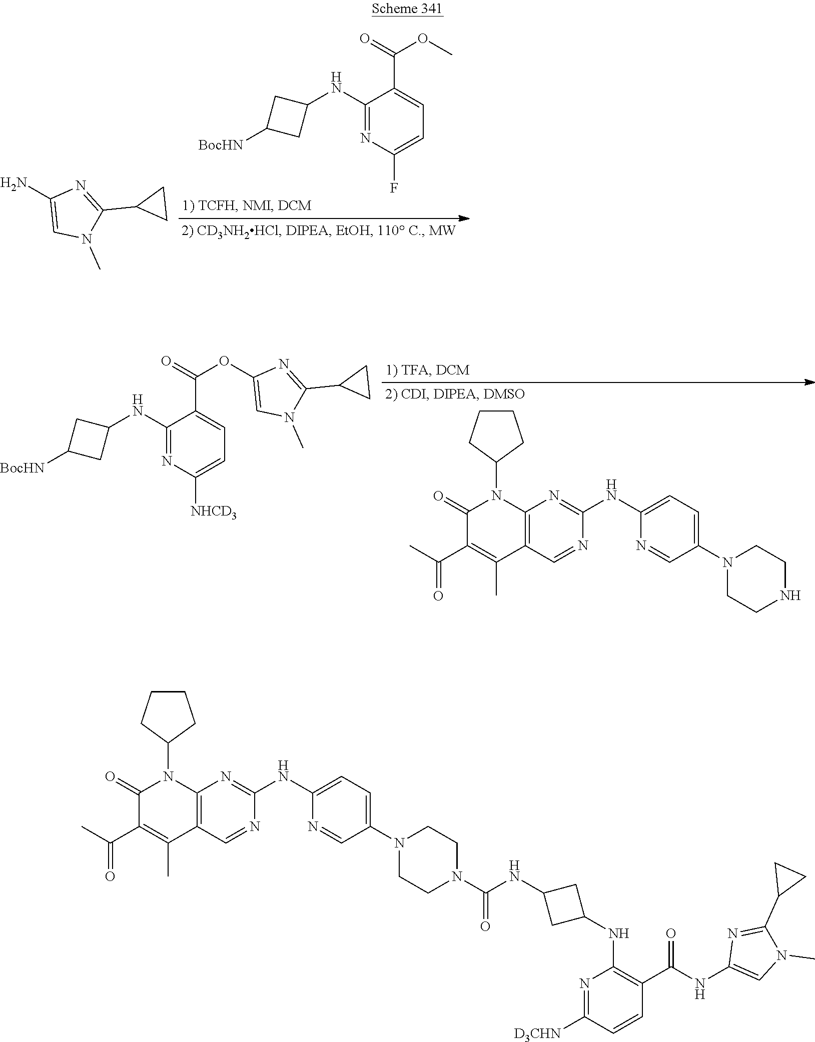

### Step 1. Synthesis of tert-butyl (3-((3-((2-cyclopropyl-1-methyl-1H-imidazol-4-yl)carbamoyl)-6-fluoropyridin-2-yl)amino)cyclobutyl)carbamate

To a solution of 2-cyclopropyl-1-methyl-1H-imidazol-4-amine (75 mg, 546.72 μmol) in dichloromethane (4 mL) were added 2-((3-((tert-butoxycarbonyl)amino)cyclobutyl)amino)-6-fluoronicotinic acid (88.9 mg, 273.36 mol), TCFH (230.1 mg, 820.08 μmol) and NMI (134.7 mg, 1.64 mmol). Then the mixture was stirred at rt for 1 h. The reaction mixture was poured into water (20 mL) and extracted with EtOAc (3×20 mL). The combined organic layers were washed with saturated brine (2×30 mL), dried over anhydrous sodium sulfate, filtered, and evaporated under reduced pressure. The resulting residue was purified by silica gel chromatography to give the desired product (120 mg, 49% yield) as a yellow solid. MS (ESI) m/z=445.7 [M+H]⁺.

### Step 2. Synthesis of tert-butyl (3-((3-((2-cyclopropyl-1-methyl-1H-imidazol-4-yl)carbamoyl)-6-((methyl-d₃)amino)pyridin-2-yl)amino)cyclobutyl)carbamate

To a solution of tert-butyl (3-((3-((2-cyclopropyl-1-methyl-1H-imidazol-4-yl)carbamoyl)-6-fluoropyridin-2-yl)amino)cyclobutyl)carbamate (50 mg, 112.49 μmol) in EtOH (1 mL) were added methyl-d₃-aminehydrochloride (79.6 mg, 1.12 mmol) and DIPEA (290.8 mg, 2.25 mmol). Then the mixture was stirred at 110° C. for 5 h under microwave irradiation. The reaction mixture was poured into water (10 mL) and extracted with dichloromethane (3×10 mL). The combined organic layers were washed with saturated brine (2×15 mL), dried over anhydrous sodium sulfate, filtered, and evaporated under reduced pressure. The resulting residue was purified by silica gel chromatography to give the desired product (48 mg, 93% yield) as a yellow solid. MS (ESI) m/z=459.4 [M+H]⁺.

### Step 3. Synthesis of 2-((3-aminocyclobutyl)amino)-N-(2-cyclopropyl-1-methyl-1H-imidazol-4-yl)-6-((methyl-d₃)amino)nicotinamide

To a solution of tert-butyl (3-((3-((2-cyclopropyl-1-methyl-1H-imidazol-4-yl)carbamoyl)-6-((methyl-d3)amino)pyridin-2-yl)amino)cyclobutyl)carbamate (48 mg, 104.67 μmol) in dichloromethane (2 mL) was added TFA (1 mL). Then the mixture was stirred at rt for 1 h. The reaction mixture was evaporated under reduced pressure to give the crude product (36 mg, 96% yield) as a colorless oil, which was used directly to the next step without further purification. MS (ESI) m/z=359.3 [M+H]⁺.

### Step 4. Synthesis of 4-(6-((6-acetyl-8-cyclopentyl-5-methyl-7-oxo-7,8-dihydropyrido[2,3-d]pyrimidin-2-yl)amino)pyridin-3-yl)-N-(3-((3-((2-cyclopropyl-1-methyl-1H-imidazol-4-yl)carbamoyl)-6-((methyl-d₃)amino)pyridin-2-yl)amino)cyclobutyl)piperazine-1-carboxamide

To a solution of 2-((3-aminocyclobutyl)amino)-N-(2-cyclopropyl-1-methyl-1H-imidazol-4-yl)-6-((methyl-d3)amino)nicotinamide (18 mg, 50.22 gmol) in DMSO (1 mL) were added CDI (7.2 mg, 50.22 μmol) and DIPEA (13.0 mg, 100.43 gmol) at rt. The mixture was stirred at rt for 1 h. Then 6-acetyl-8-cyclopentyl-5-methyl-2-((5-(piperazin-1-yl)pyridin-2-yl)amino)pyrido[2,3-d]pyrimidin-7(8H)-one (22.5 mg, 50.22 μmol) was added and the mixture was stirred at rt for 8 h. The mixture was purified by pre-HPLC to give the desired product (8.1 mg, 19% yield) as a yellow solid. MS (ESI) m/z=832.8 [M+H]⁺.

### Example 342. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-(isopropylamino)nicotinamide (CPD-288)

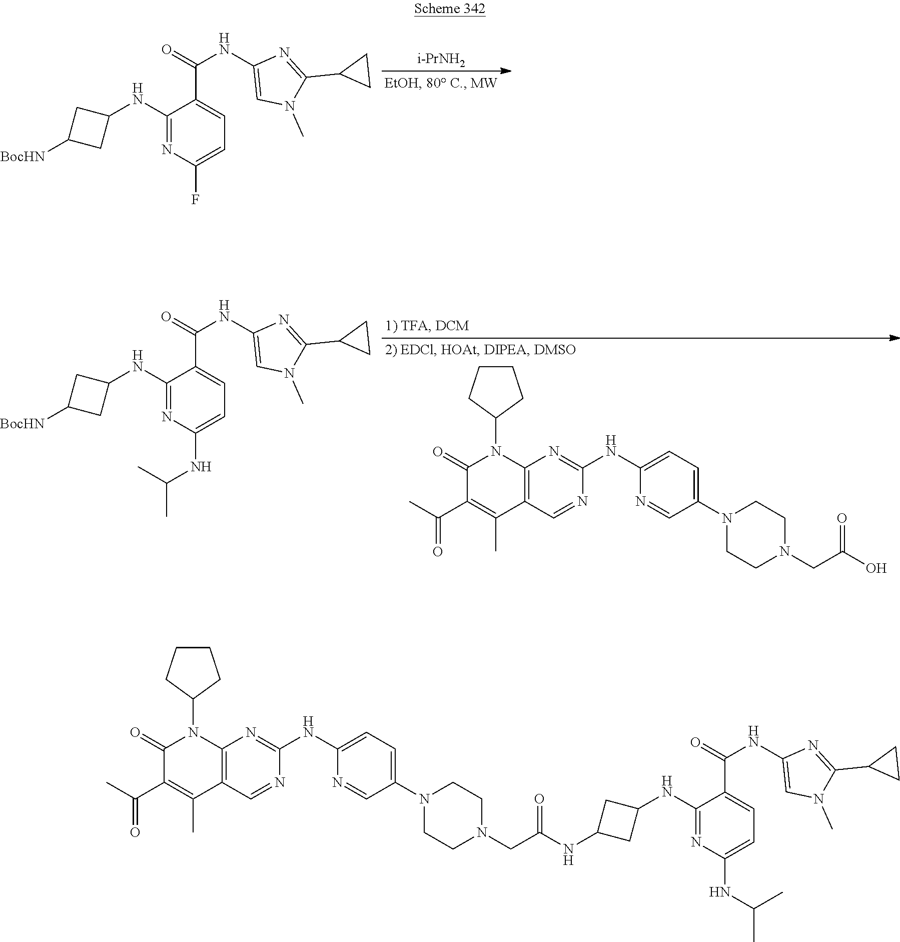 [SMILES: "i-PrNH2" ; Cn1cc(NC(=O)c2ccc(F)nc2NC2CC(NC(=O)OC(C)(C)C)C2)nc1C1CC1 ; CC(C)Nc1ccc(C(=O)Nc2cn(C)c(C3CC3)n2)c(NC2CC(NC(=O)OC(C)(C)C)C2)n1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NC5CC(Nc6nc(NC(C)C)ccc6C(=O)Nc6cn(C)c(C7CC7)n6)C5)CC4)cn3)nc2n(C2CCCC2)c1=O]

CPD-288 was synthesized following the standard procedures for preparing CPD-144 (4.4 mg, 5% yield over 3 steps) as a yellow solid. MS (ESI) m/z=871.6 [M+H]⁺.

### Example 343. 4-(6-((6-acetyl-8-cyclopentyl-5-methyl-7-oxo-7,8-dihydropyrido[2,3-d]pyrimidin-2-yl)amino)pyridin-3-yl)-N-(3-((3-((2-cyclopropyl-1-methyl-1H-imidazol-4-yl)carbamoyl)-6-(isopropylamino)pyridin-2-yl)amino)cyclobutyl)piperazine-1-carboxamide (CPD-289)

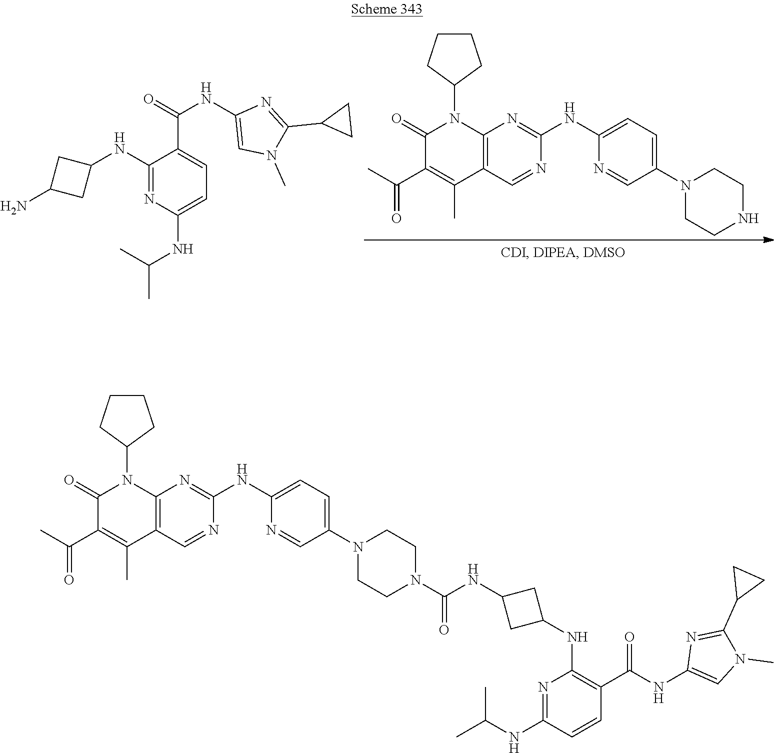

CPD-289 was synthesized following the standard procedure for preparing CPD-287 (6.5 mg, 15% yield) as a yellow solid. MS (ESI) m/z=857.6 [M+H]⁺.

### Example 344. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-6-(isopropylamino)-N-(5-methylisoxazol-3-yl)nicotinamide (CPD-290)

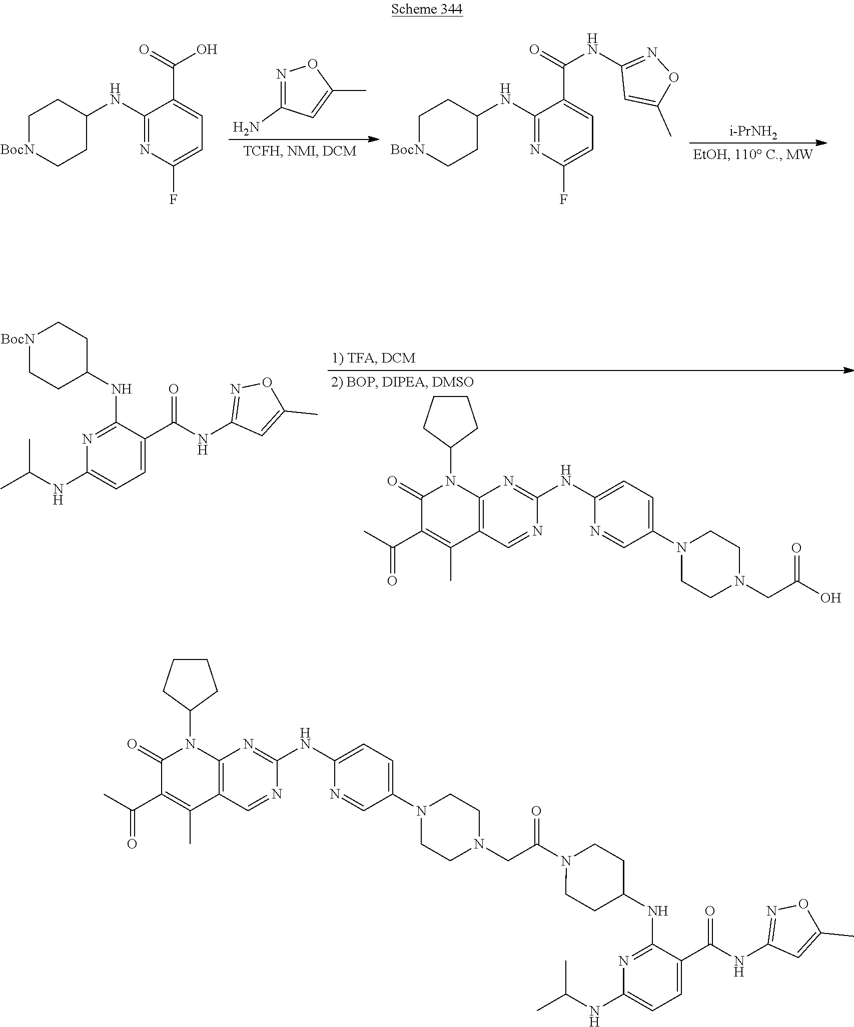

CPD-290 was synthesized following the standard procedures for preparing CPD-144 (7.0 mg, 7% yield over 4 steps) as a yellow solid. MS (ESI) m/z=846.7 [M+H]⁺.

### Example 345. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(dimethylamino)nicotinamide (CPD-291)

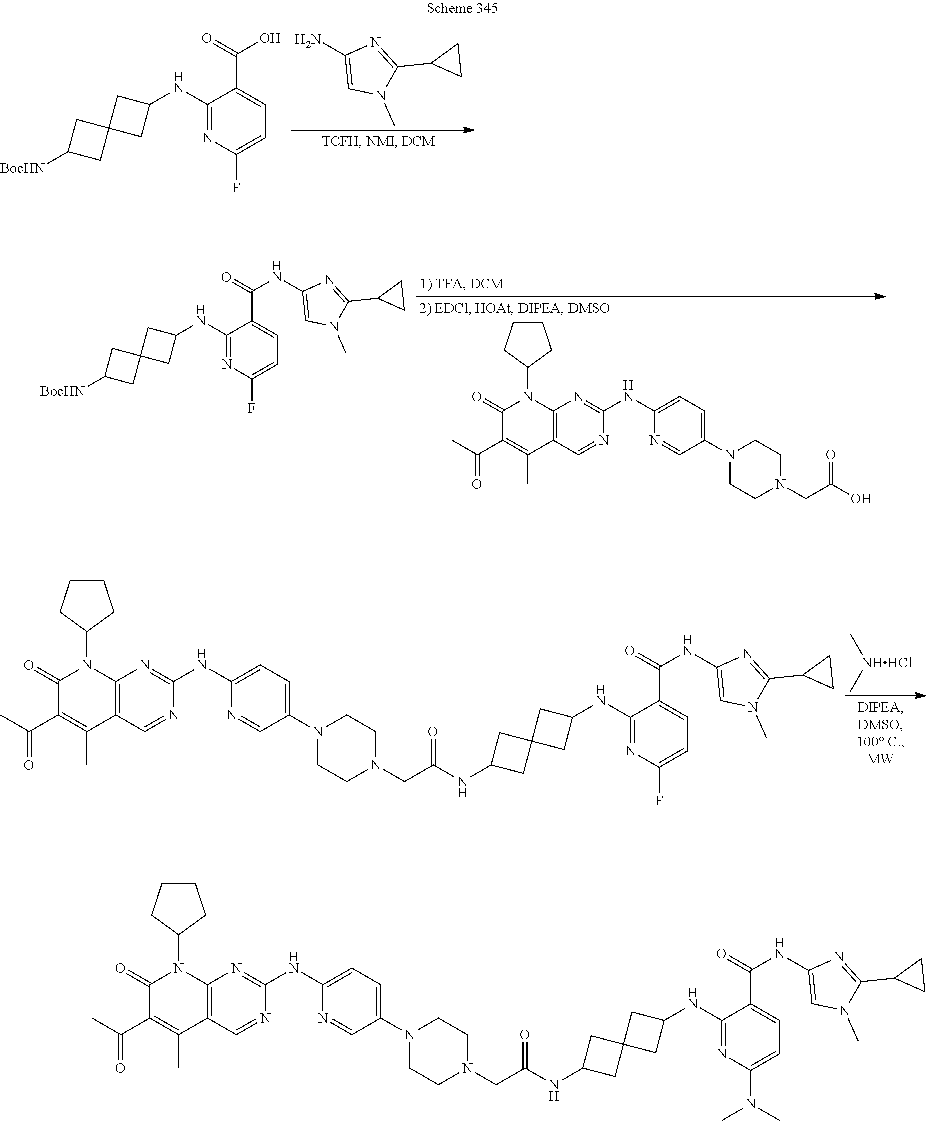

CPD-291 was synthesized following the standard procedures for preparing CPD-144 and CPD-253 (8.0 mg, 6% yield over 4 steps) as a yellow solid. MS (ESI) m/z=897.6 [M+H]⁺.

### Example 346. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-isopropoxynicotinamide (CPD-292)

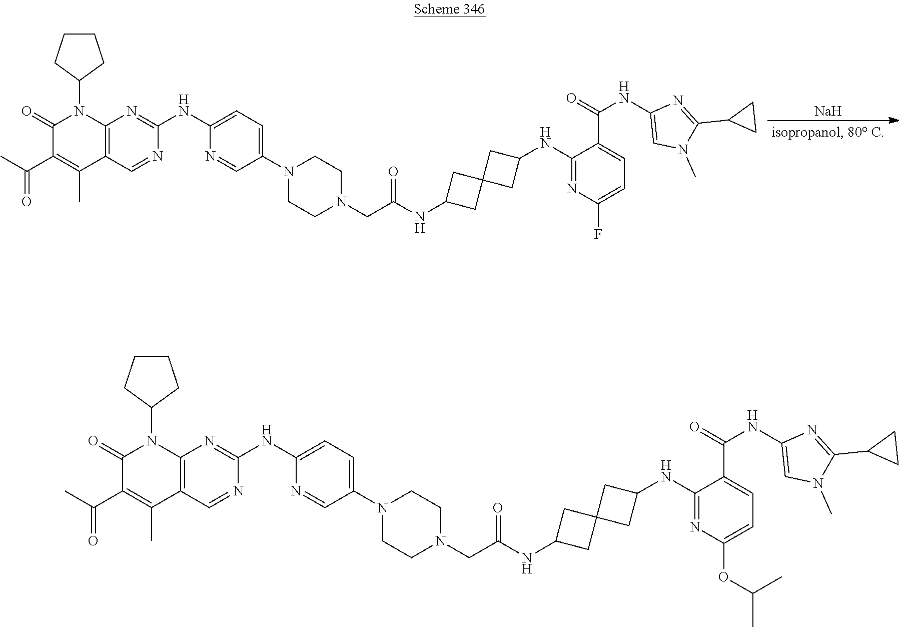

To a solution of NaH (11 mg, 0.46 mmol) in isopropanol (5 mL) was added 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-fluoronicotinamide (40 mg, 45.87 μmol). The mixture was stirred at 80° C. for 6 h, before it was poured into water (30 mL) and extracted with ethyl acetate (2×40 mL). The combined organic layers were washed with saturated brine (30 mL), dried over anhydrous sodium sulfate, filtered, and evaporated under reduced pressure. The residue was purified by reverse-phase chromatography to get the product (3.0 mg, 7% yield) as a yellow solid. MS (ESI) m/z=912.7 [M+H]⁺.

### Example 347. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(ethylamino)nicotinamide (CPD-293)

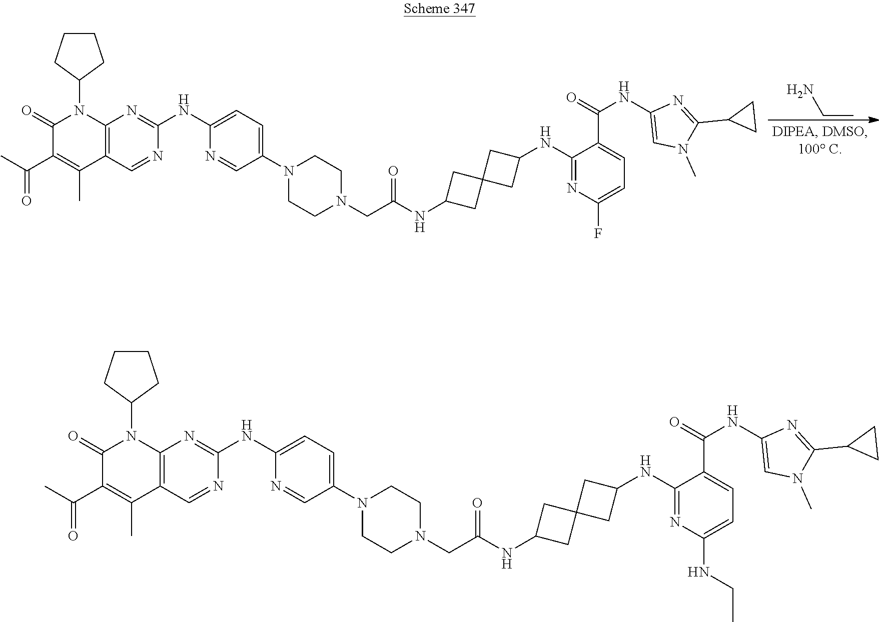

To a solution of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-fluoronicotinamide (20 mg, 22.94 μmol) in DMSO (2 mL) were added ethanamine (10 mg, 0.23 mmol) and DIPEA (30 mg, 0.23 mmol). The mixture was stirred at 100° C. for 2 h, before it was purified by prep-HPLC to get the product (8 mg, 39% yield) as a yellow solid. MS (ESI) m/z=897.7 [M+H]⁺.

### Example 348. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-6-((methyl-d3)amino)-N-(5-methylisoxazol-3-yl)nicotinamide (CPD-294)

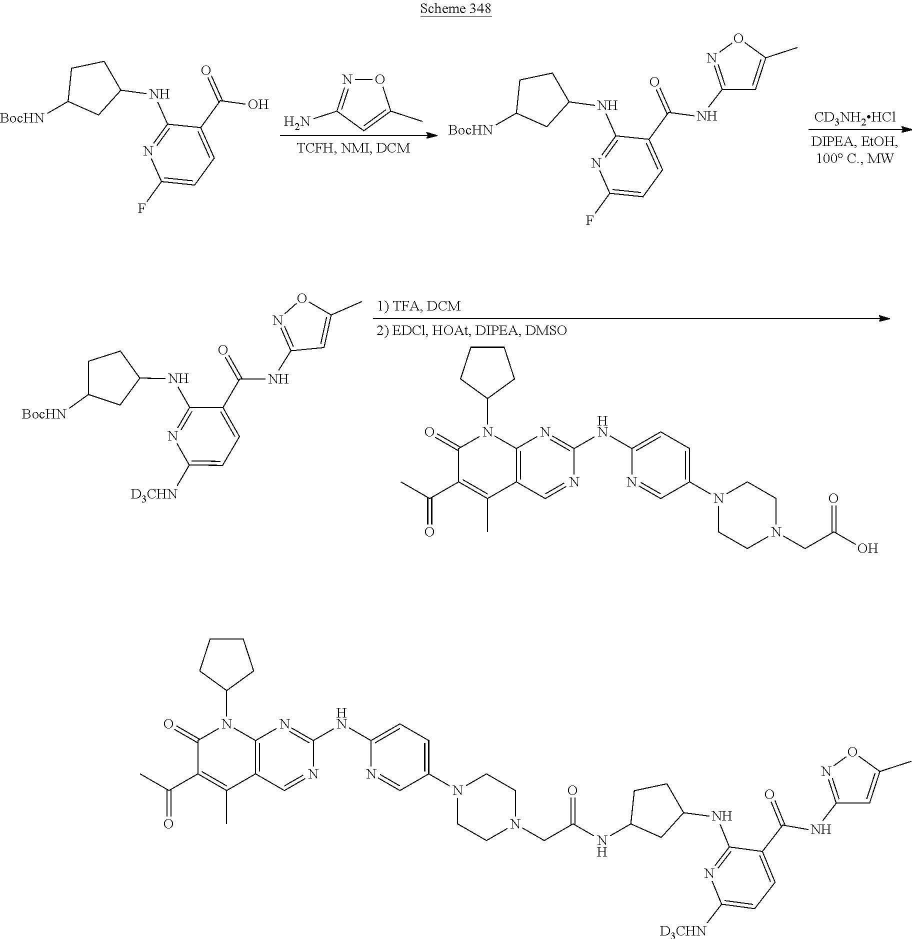

CPD-294 was synthesized following the standard procedures for preparing CPD-144 (5.0 mg, 7% yield over 4 steps) as a yellow solid. MS (ESI) m/z=821.7 [M+H]⁺.

### Example 349. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclopentyl)amino)-6-(isopropylamino)-N-(5-methylisoxazol-3-yl)nicotinamide (CPD-295)

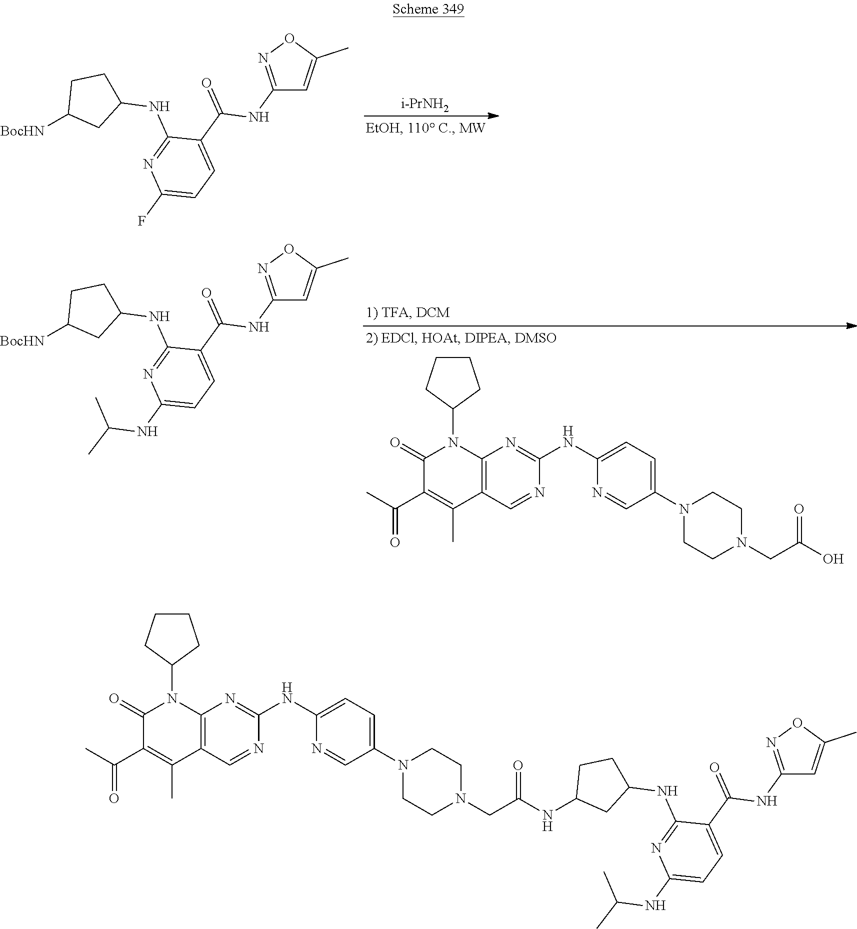 [SMILES: "i-PrNH2" ; Cc1cc(NC(=O)c2ccc(F)nc2NC2CCC(NC(=O)OC(C)(C)C)C2)no1 ; Cc1cc(NC(=O)c2ccc(NC(C)C)nc2NC2CCC(NC(=O)OC(C)(C)C)C2)no1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NC5CCC(Nc6nc(NC(C)C)ccc6C(=O)Nc6cc(C)on6)C5)CC4)cn3)nc2n(C2CCCC2)c1=O]

CPD-295 was synthesized following the standard procedures for preparing CPD-144 (2.4 mg, 6% yield over 3 steps) as a yellow solid. MS (ESI) m/z=846.6 [M+H]⁺.

### Example 350. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopentyl-1-methyl-1H-imidazol-4-yl)-6-(methylamino)nicotinamide (CPD-296)

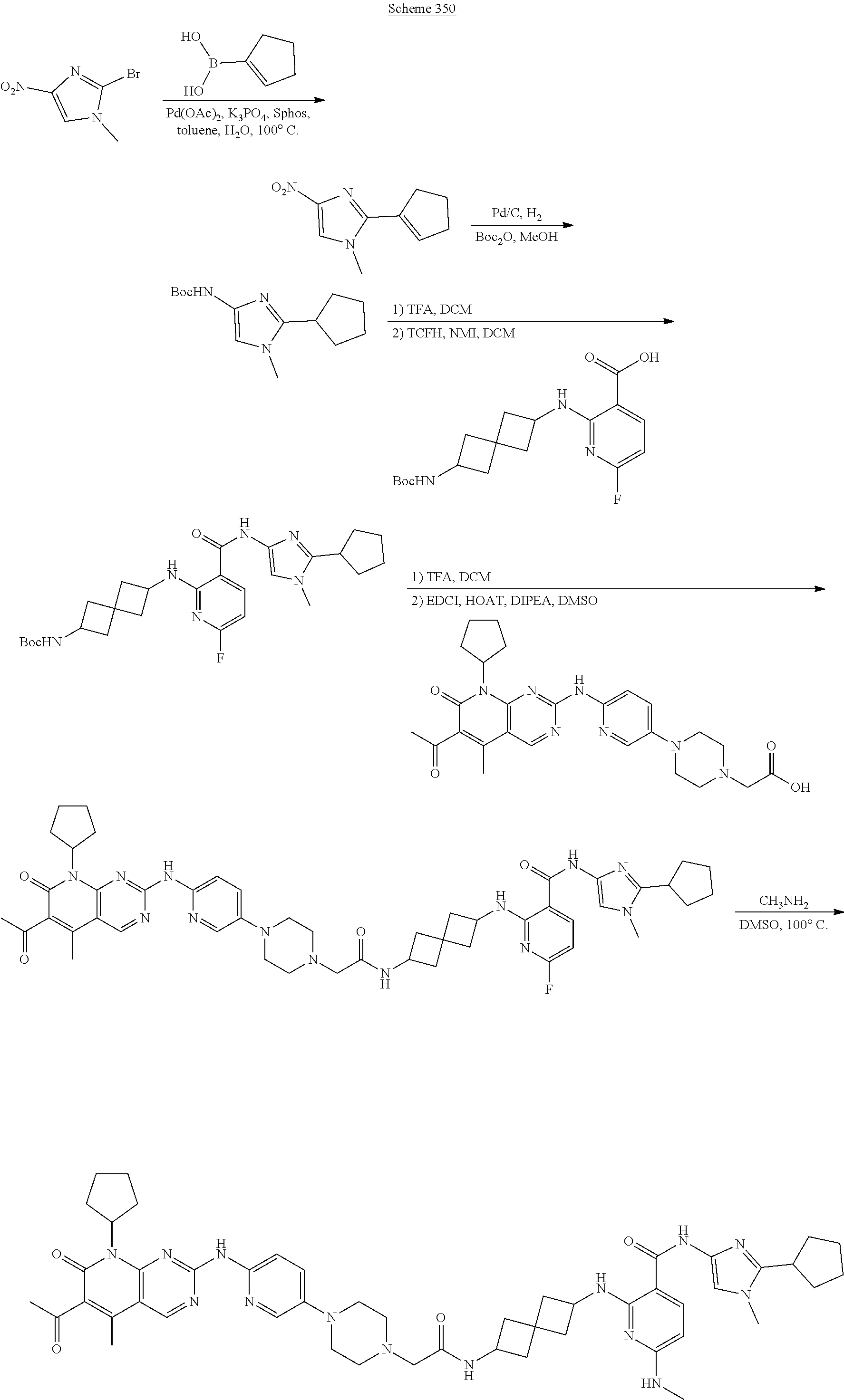

### Step 1. Synthesis of 2-(cyclopent-1-en-1-yl)-1-methyl-4-nitro-1H-imidazole

To a mixture of 2-bromo-1-methyl-4-nitro-1H-imidazole (35 mg, 0.17 mmol) and cyclopent-1-en-1-ylboronic acid (164 mg, 0.8 mmol) in toluene (5 mL) and H₂O (1 mL) were added K₃PO₄ (108 mg, 0.5 mmol), Sphos (7 mg, 0.01 mmol) and Pd(OAc)₂ (4 mg, 0.01 mmol) at room temperature under N₂. The reaction mixture was stirred at 100° C. for 16 h. The mixture was concentrated and the resulting residue was purified by silica gel flash chromatography to give the desired product (26 mg, 79% yield) as a colorless oil. MS (ESI) m/z=194.2 [M+H]⁺.

### Step 2. Synthesis of tert-butyl (2-cyclopentyl-1-methyl-1H-imidazol-4-yl)carbamate

To a solution of 2-(cyclopent-1-en-1-yl)-1-methyl-4-nitro-1H-imidazole (26 mg, 0.13 mmol) in MeOH (5 mL) were added BOC₂O (587 mg, 2.69 mmol) and 10% Pd/C (14 mg). The reaction mixture was stirred at rt for 3 h under hydrogen balloon. The mixture was filtered and concentrated. The resulting residue was purified by silica gel flash chromatography to give the desired product (21 mg, 58% yield) as a white solid. MS (ESI) m/z=266.5 [M+H]⁺.

### Step 3. Synthesis of 2-cyclopentyl-1-methyl-1H-imidazol-4-amine

To a solution of tert-butyl (2-cyclopentyl-1-methyl-1H-imidazol-4-yl)carbamate (21 mg, 0.08 mmol) in dichloromethane (2 mL) was added TFA (1 mL). The reaction mixture was stirred at rt for 1 h. The mixture was concentrated to give the desired product (21 mg, 95% yield) as a colorless oil. This compound was used directly in the next step without further purification. MS (ESI) m/z=166.2 [M+H]⁺.

### Step 4. Synthesis of tert-butyl (6-((3-((2-cyclopentyl-1-methyl-1H-imidazol-4-yl)carbamoyl)-6-fluoropyridin-2-yl)amino)spiro[3.3]heptan-2-yl)carbamate

A mixture of 2-((6-((tert-butoxycarbonyl)amino)spiro[3.3]heptan-2-yl)amino)-6-fluoronicotinic acid (32 mg, 0.09 mmol), 2-cyclopentyl-1-methyl-1H-imidazol-4-amine (25 mg, 0.09 mmol), TCFH (50 mg, 0.18 mmol) and NMI (37 mg, 0.45 mmol) in dichloromethane (5 mL) was stirred at rt for 1 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by silica gel flash chromatography to give the desired product (18 mg, 39% yield) as a colorless oil. MS (ESI) m/z=513.5 [M+H]⁺.

### Step 5. Synthesis of 2-((6-aminospiro[3.3]heptan-2-yl)amino)-N-(2-cyclopentyl-1-methyl-1H-imidazol-4-yl)-6-fluoronicotinamide

To a solution of tert-butyl (6-((3-((2-cyclopentyl-1-methyl-1H-imidazol-4-yl)carbamoyl)-6-fluoropyridin-2-yl)amino)spiro[3.3]heptan-2-yl)carbamate (18 mg, 0.03 mmol) in dichloromethane (2 mL) was added TFA (1 mL). The reaction mixture was stirred at rt for 1 h. The mixture was concentrated to give the desired product (18 mg, 97% yield) as a colorless oil. This compound was used directly in the next step without further purification. MS (ESI) m/z=413.4 [M+H]⁺.

### Step 6. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopentyl-1-methyl-1H-imidazol-4-yl)-6-fluoronicotinamide

A mixture of 2-((6-aminospiro[3.3]heptan-2-yl)amino)-N-(2-cyclopentyl-1-methyl-1H-imidazol-4-yl)-6-fluoronicotinamide (18 mg, 0.03 mmol), 5-((3-((6-(dimethylamino)pyridazin-3-yl)carbamoyl)-6-(methylamino)pyridin-2-yl)amino)pentanoic acid (17 mg, 0.03 mmol), EDCI (9.8 mg, 0.05 mmol), HOAT (6.9 mg, 0.05 mmol) and DIPEA (22 mg, 0.17 mmol) in DMSO (2 mL) was stirred at rt for 16 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by pre-TLC to give the desired product (16 mg, 52% yield) as a yellow solid. MS (ESI) m/z=900.6 [M+H]⁺.

### Step 7. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopentyl-1-methyl-1H-imidazol-4-yl)-6-(methylamino)nicotinamide

To a mixture of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopentyl-1-methyl-1H-imidazol-4-yl)-6-fluoronicotinamide (16 mg, 0.01 mmol) in DMSO (1 mL) was added methanamine (9 mg, 0.1 mmol, 30% in EtOH). The reaction mixture was stirred at 100° C. for 1 h under microwave irradiation. The reaction mixture was purified by reverse phase chromatography to give the desired product (1.1 mg, 6% yield) as a yellow solid. MS (ESI) m/z=911.7 [M+H]⁺.

### Example 351. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-cyclobutyl-5-methyl-1H-pyrazol-3-yl)-6-(methylamino)nicotinamide (CPD-297)

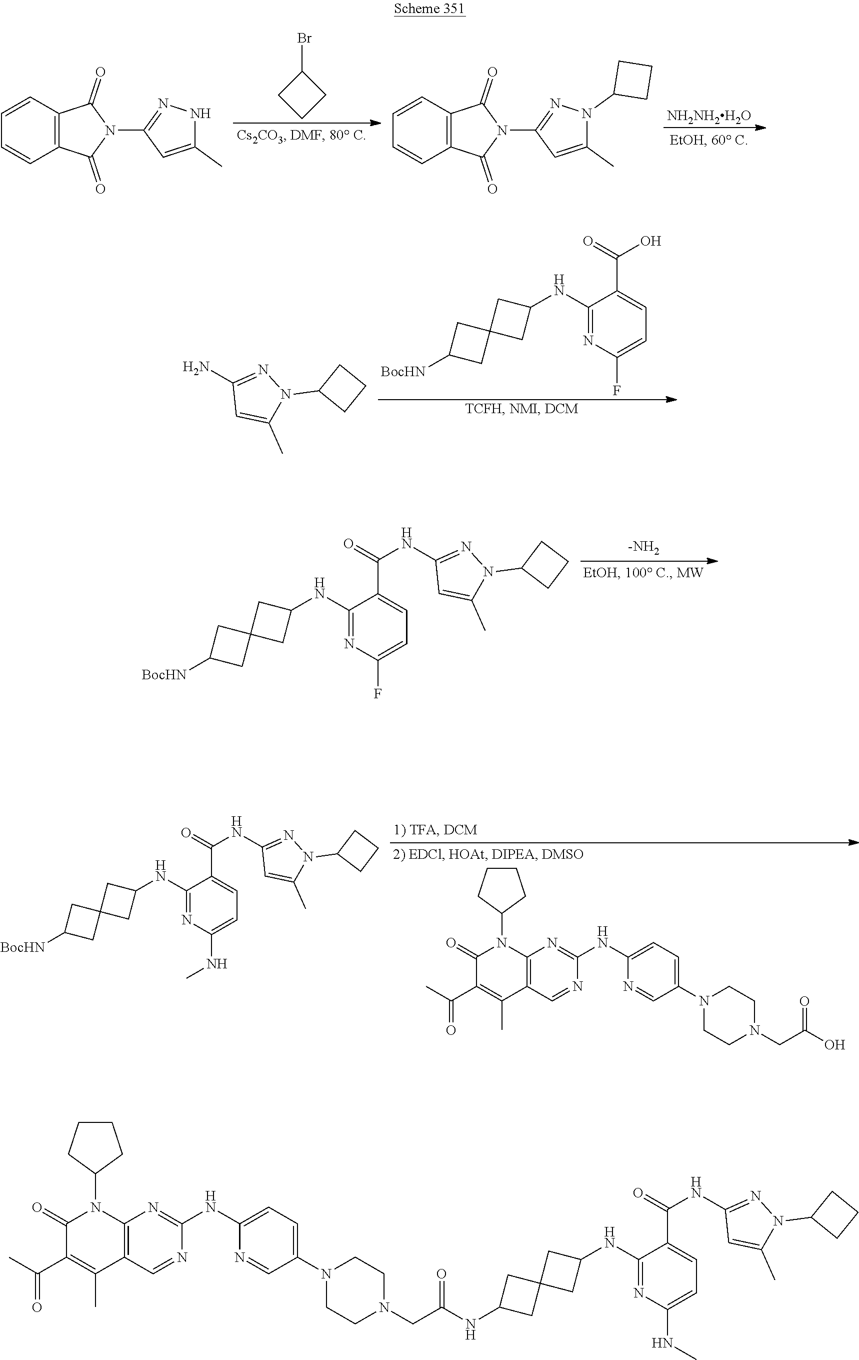

### Step 1. Synthesis of 2-(1-cyclobutyl-5-methyl-1H-pyrazol-3-yl)isoindoline-1,3-dione

To a solution of 2-(5-methyl-1H-pyrazol-3-yl)isoindoline-1,3-dione (600 mg, 2.64 mmol) in DMF (15 mL) were added bromocyclobutane (1.07 g, 7.92 mmol) and Cs₂CO₃ (4.30 g, 13.20 mmol). The mixture was stirred at 80° C. for 4 h, before it was poured into water (50 mL) and extracted with ethyl acetate (2×50 mL). The combined organic extracts were washed with saturated brine (50 mL), dried over anhydrous sodium sulfate, filtered, and evaporated under reduced pressure. The residue was purified by silica gel chromatography to get the product (50 mg, 7% yield) as a light-yellow solid. MS (ESI) m/z=282.5 [M+H]⁺.

### Step 2. Synthesis of 1-cyclobutyl-5-methyl-1H-pyrazol-3-amine

To a solution of 2-(1-cyclobutyl-5-methyl-1H-pyrazol-3-yl)isoindoline-1,3-dione (50 mg, 0.18 mmol) in EtOH (5 mL) was added hydrazine hydrate (1 mL). The mixture was stirred at 60° C. for 2 h, before it was poured into water (20 mL) and extracted with ethyl acetate (3×30 mL). The combined organic extracts were washed with saturated brine (30 mL), dried over anhydrous sodium sulfate, filtered, and evaporated under reduced pressure. The residue was purified by silica gel chromatography (25 mg, 93% yield) as a colorless solid. MS (ESI) m/z=152.3 [M+H]⁺. The remaining steps were performed according to the procedures for preparing CPD-144 to provide the desired product (7 mg, 11% yield over 4 steps) as a yellow solid. MS (ESI) m/z=897.6 [M+H]⁺.

### Example 352. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(propylamino)nicotinamide (CPD-298)

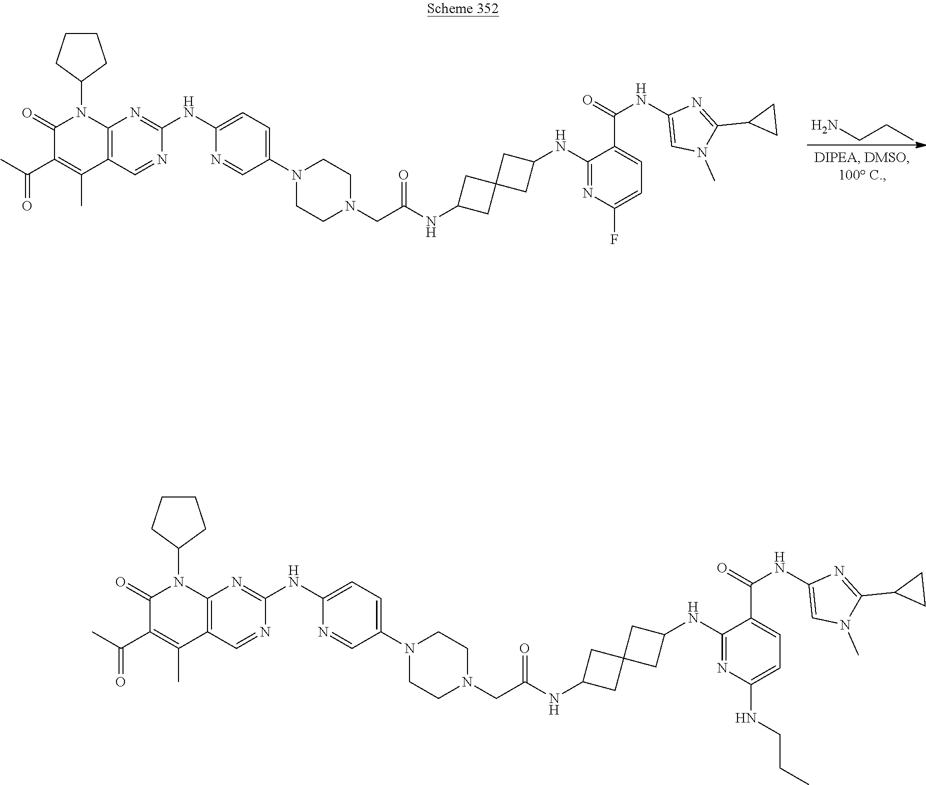

CPD-298 was synthesized following the standard procedure for preparing CPD-293 (10.0 mg, 48% yield) as a yellow solid. MS (ESI) m/z=911.7 [M+H]⁺.

### Example 353. 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)piperidin-4-yl)amino)-N-(6-(dim ethylamino)pyridazin-3-yl)-6-(methylamino)nicotinamide (CPD-299)

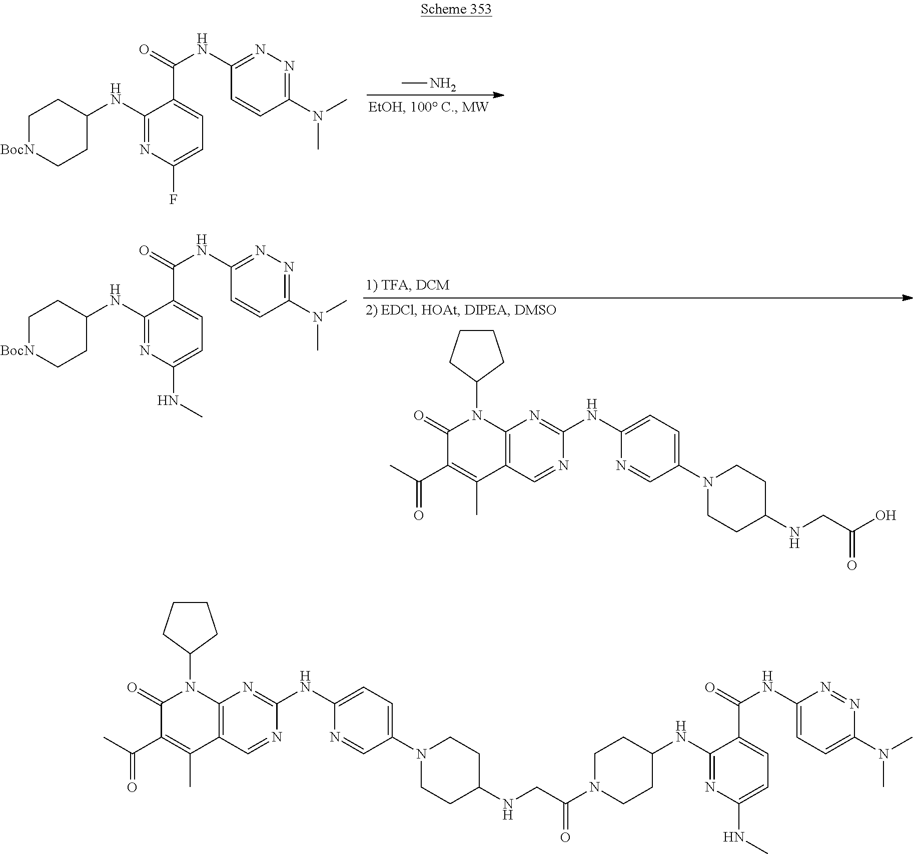

CPD-299 was synthesized following the standard procedures for preparing CPD-144 (10.0 mg, 14% yield over 3 steps) as a yellow solid. MS (ESI) m/z=872.5 [M+H]⁺.

### Example 354. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-(methylamino)nicotinamide (CPD-300)

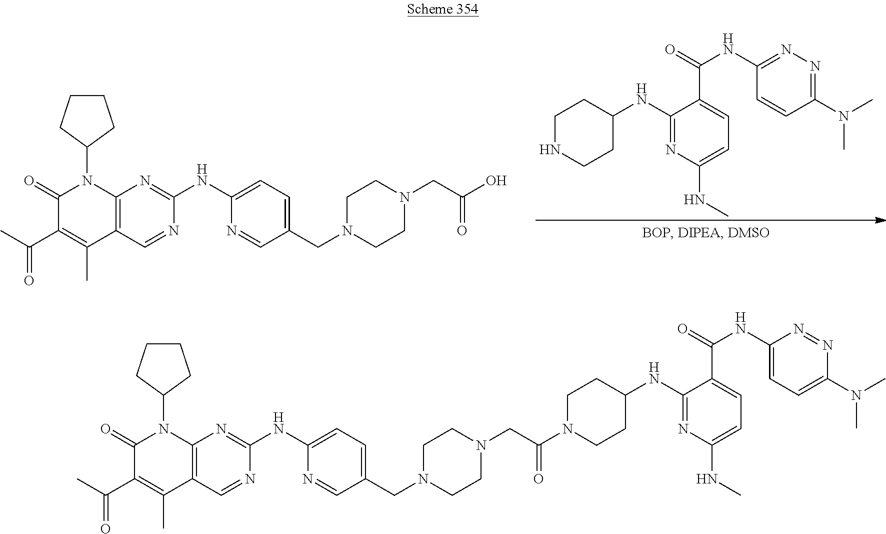 [SMILES: CC(=O)c1c(C)c2cnc(Nc3ccc(CN4CCN(CC(=O)O)CC4)cn3)nc2n(C2CCCC2)c1=O ; CNc1ccc(C(=O)Nc2ccc(N(C)C)nn2)c(NC2CCNCC2)n1 ; CNc1ccc(C(=O)Nc2ccc(N(C)C)nn2)c(NC2CCN(C(=O)CN3CCN(Cc4ccc(Nc5ncc6c(C)c(C(C)=O)c(=O)n(C7CCCC7)c6n5)nc4)CC3)CC2)n1]

CPD-300 was synthesized following the standard procedure for preparing CPD-261 (10.0 mg, 21% yield) as a light-yellow solid. MS (ESI) m/z=872.5 [M+H]⁺.

### Example 355. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(6-(dimethylamino)pyridazin-3-yl)-6-(isopropylamino)nicotinamide (CPD-301)

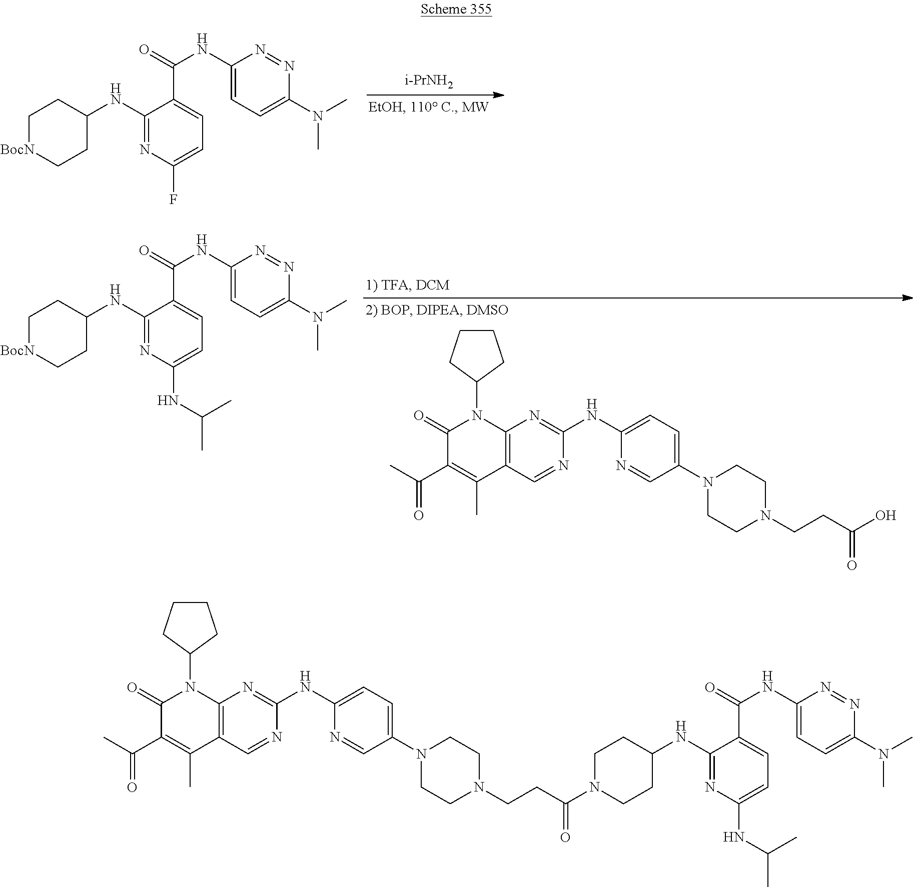 [SMILES: CN(C)c1ccc(NC(=O)c2ccc(F)nc2NC2CCN(C(=O)OC(C)(C)C)CC2)nn1 ; CC(C)Nc1ccc(C(=O)Nc2ccc(N(C)C)nn2)c(NC2CCN(C(=O)OC(C)(C)C)CC2)n1 ; CC(=O)c1c(C)c2cnc(Nc3ccc(N4CCN(CCC(=O)O)CC4)cn3)nc2n(C2CCCC2)c1=O ; CC(=O)c1c(C)c2cnc(Nc3ccc(N4CCN(CCC(=O)N5CCC(Nc6nc(NC(C)C)ccc6C(=O)Nc6ccc(N(C)C)nn6)CC5)CC4)cn3)nc2n(C2CCCC2)c1=O]

CPD-301 was synthesized following the standard procedures for preparing CPD-144 (9.0 mg, 20% yield over 3 steps) as a yellow solid. MS (ESI) m/z=900.6 [M+H]⁺.

### Example 356. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(1,5-dimethyl-1H-pyrazol-3-yl)-6-(isopropylamino)nicotinamide (CPD-302)

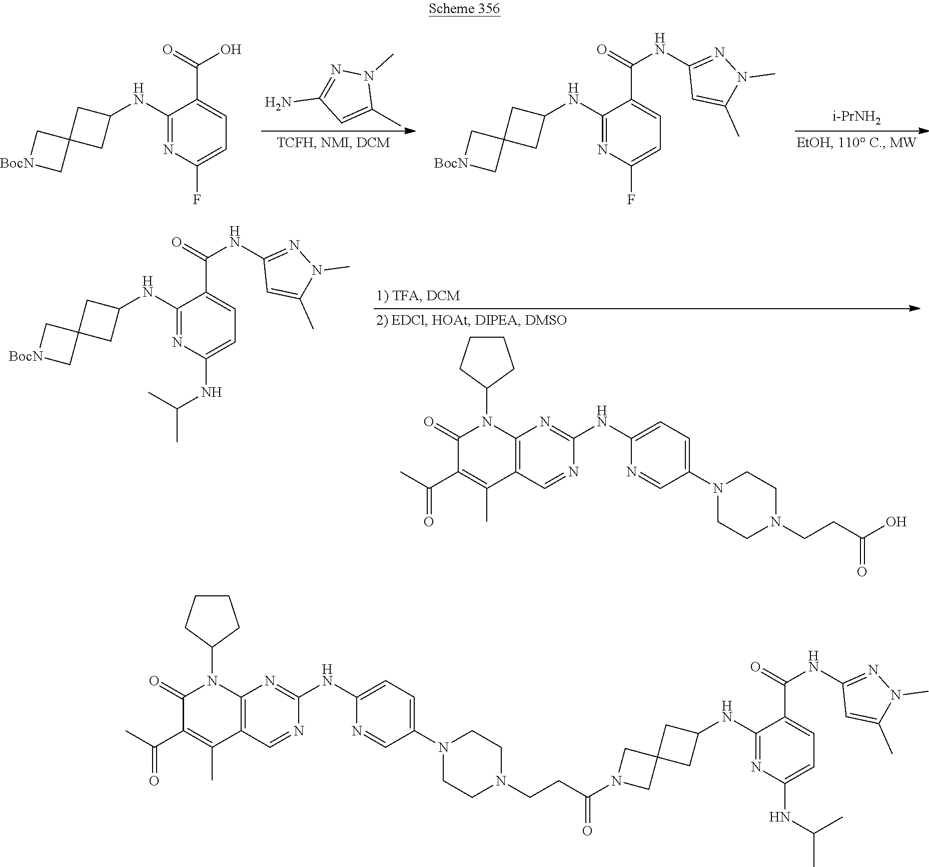

CPD-302 was synthesized following the standard procedures for preparing CPD-144 (2.6 mg, 4% yield over 4 steps) as a yellow solid. MS (ESI) m/z=885.7 [M+H]⁺.

### Example 357. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(6-(dimethylamino)pyridazin-3-yl)-6-(isopropylamino)nicotinamide (CPD-303)

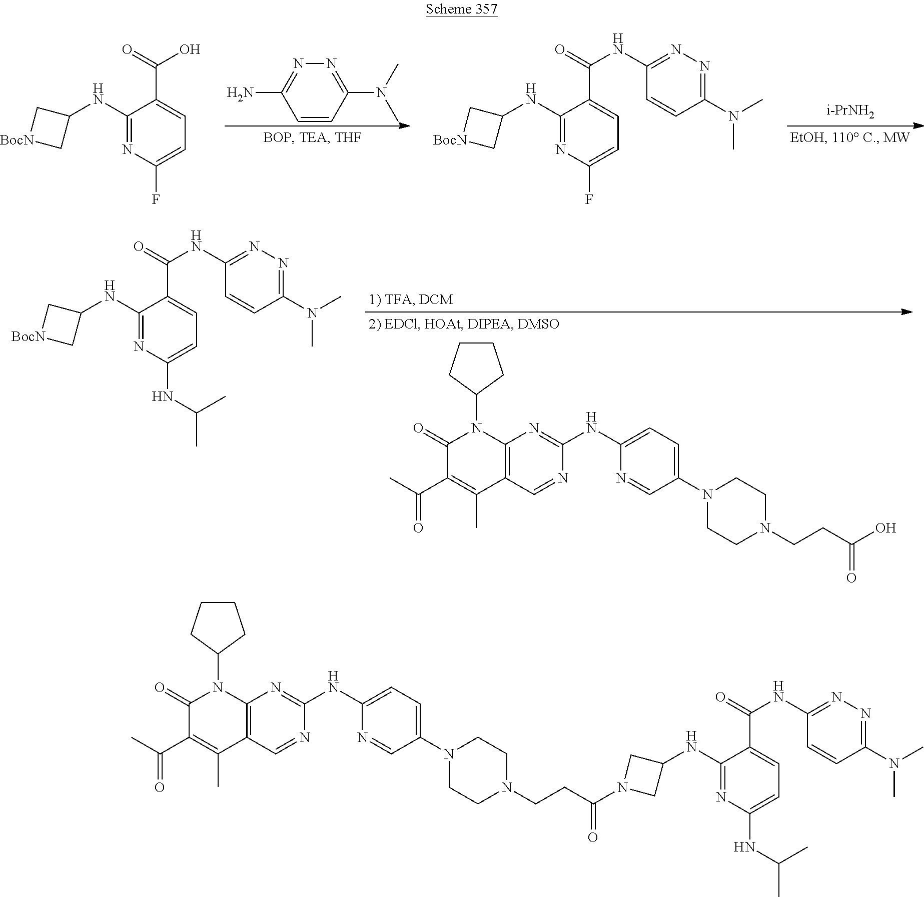

CPD-303 was synthesized following the standard procedures for preparing CPD-145 (3.0 mg, 7% yield over 4 steps) as a yellow solid. MS (ESI) m/z=872.6 [M+H]⁺.

### Example 358. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)azetidin-3-yl)amino)-N-(2-cyclopropyl-1-methyl-1H-imidazol-4-yl)-6-(isopropylamino)nicotinamide (CPD-304)

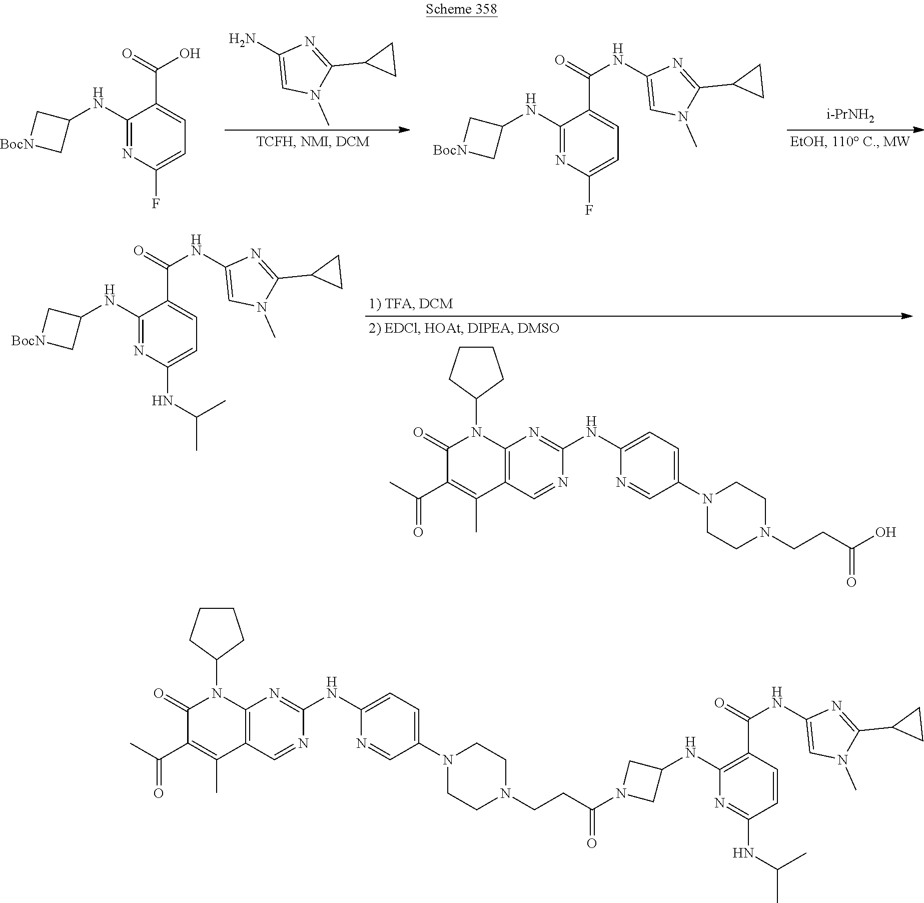

CPD-304 was synthesized following the standard procedures for preparing CPD-144 (3.0 mg, 9% yield over 4 steps) as a yellow solid. MS (ESI) m/z=871.9 [M+H]⁺.

### Example 359. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-(isopropylamino)nicotinamide (CPD-305)

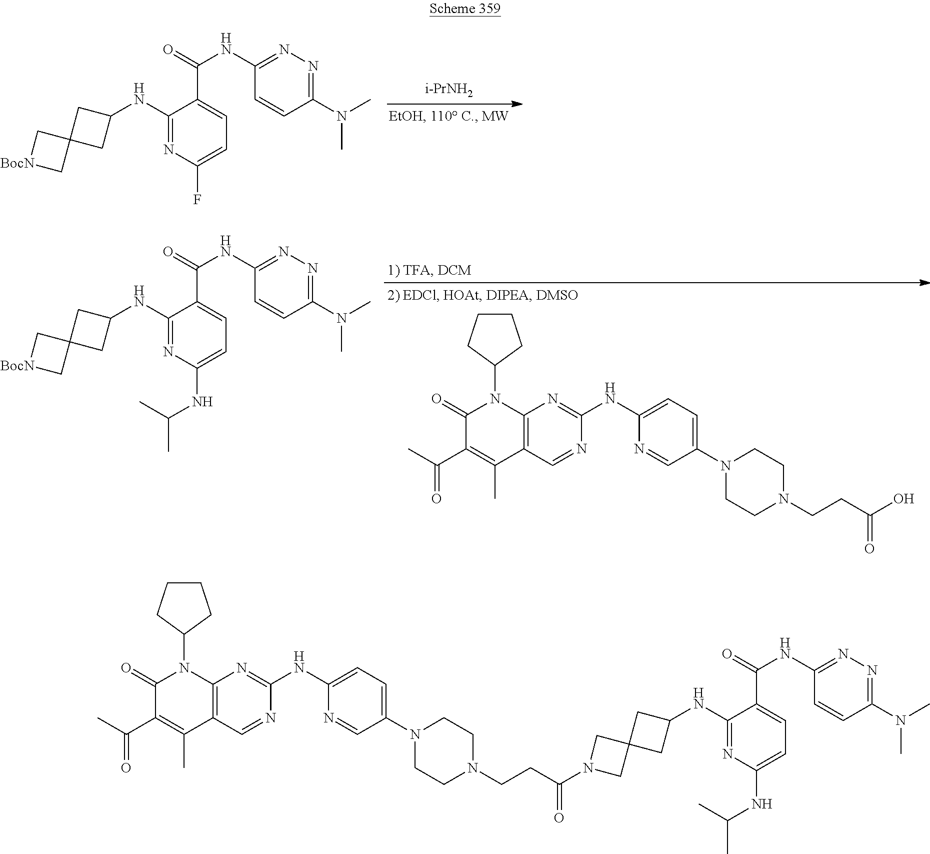 [SMILES: CN(C)c1ccc(NC(=O)c2ccc(F)nc2NC2CC3(C2)CN(C(=O)OC(C)(C)C)C3)nn1 ; CC(C)Nc1ccc(C(=O)Nc2ccc(N(C)C)nn2)c(NC2CC3(C2)CN(C(=O)OC(C)(C)C)C3)n1 ; CC(=O)c1c(C)c2cnc(Nc3ccc(N4CCN(CCC(=O)O)CC4)cn3)nc2n(C2CCCC2)c1=O ; CC(=O)c1c(C)c2cnc(Nc3ccc(N4CCN(CCC(=O)N5CC6(CC(Nc7nc(NC(C)C)ccc7C(=O)Nc7ccc(N(C)C)nn7)C6)C5)CC4)cn3)nc2n(C2CCCC2)c1=O]

CPD-305 was synthesized following the standard procedures for preparing CPD-144 (3.24 mg, 17% yield for 3 steps) as a yellow solid. MS (ESI) m/z=912.6 [M+H]⁺.

### Example 360. 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(6-(dimethylamino)pyridazin-3-yl)-6-(isopropylamino)nicotinamide (CPD-306)

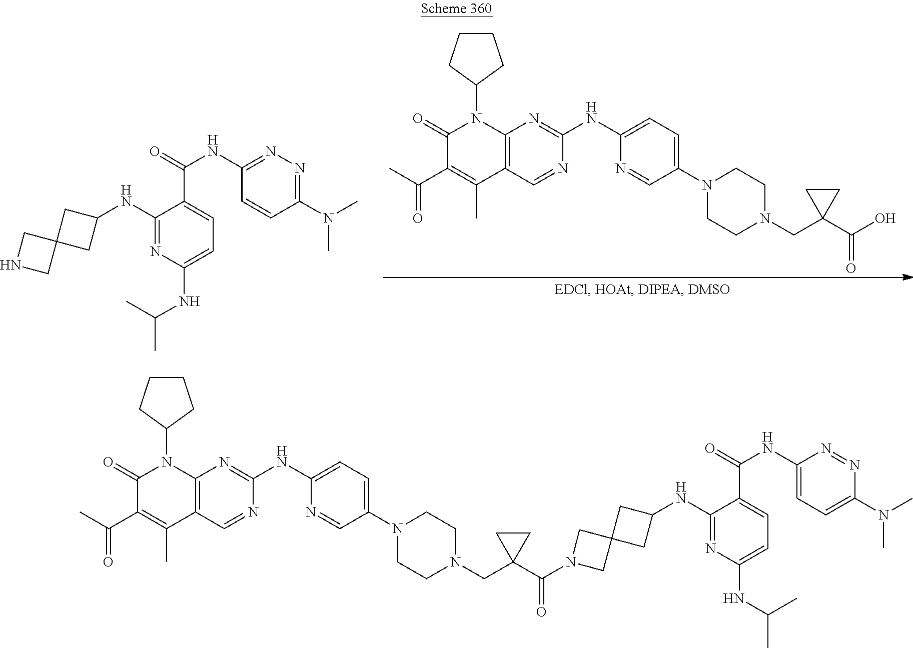

CPD-306 was synthesized following the standard procedure for preparing CPD-143 (1.19 mg, 21% yield) as a yellow solid. MS (ESI) m/z=938.7 [M+H]⁺.

### Example 361. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)cyclobutyl)amino)-N-(2-cyclopropyl-1-methyl-1H-imidazol-4-yl)-6-((methyl-d₃)amino)nicotinamide (CPD-307)

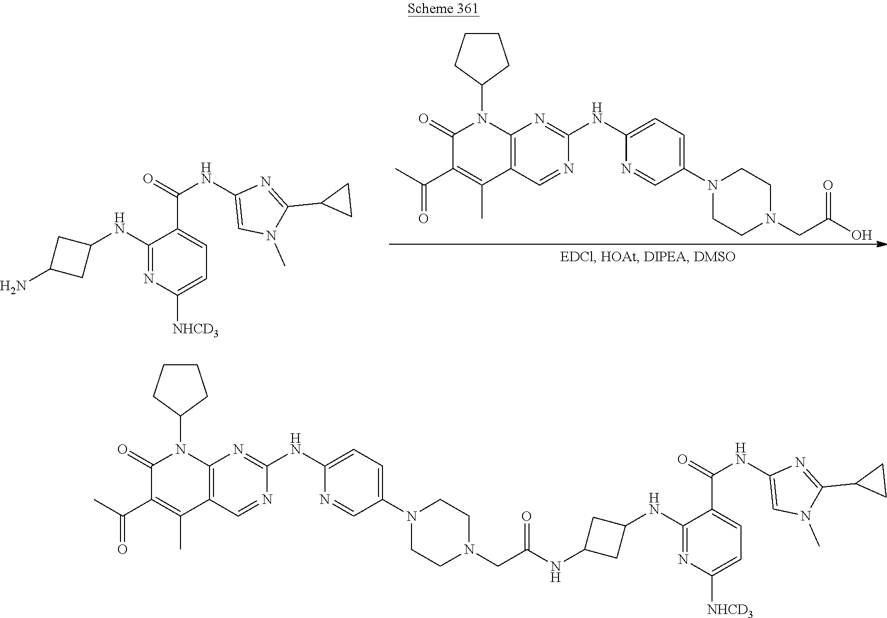

CPD-307 was synthesized following the standard procedure for preparing CPD-143 (3.0 mg, 7% yield) as a yellow solid. MS (ESI) m/z=846.6 [M+H]⁺.

### Example 362. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-ethyl-1-methyl-1H-imidazol-4-yl)-6-(methylamino)nicotinamide (CPD-308)

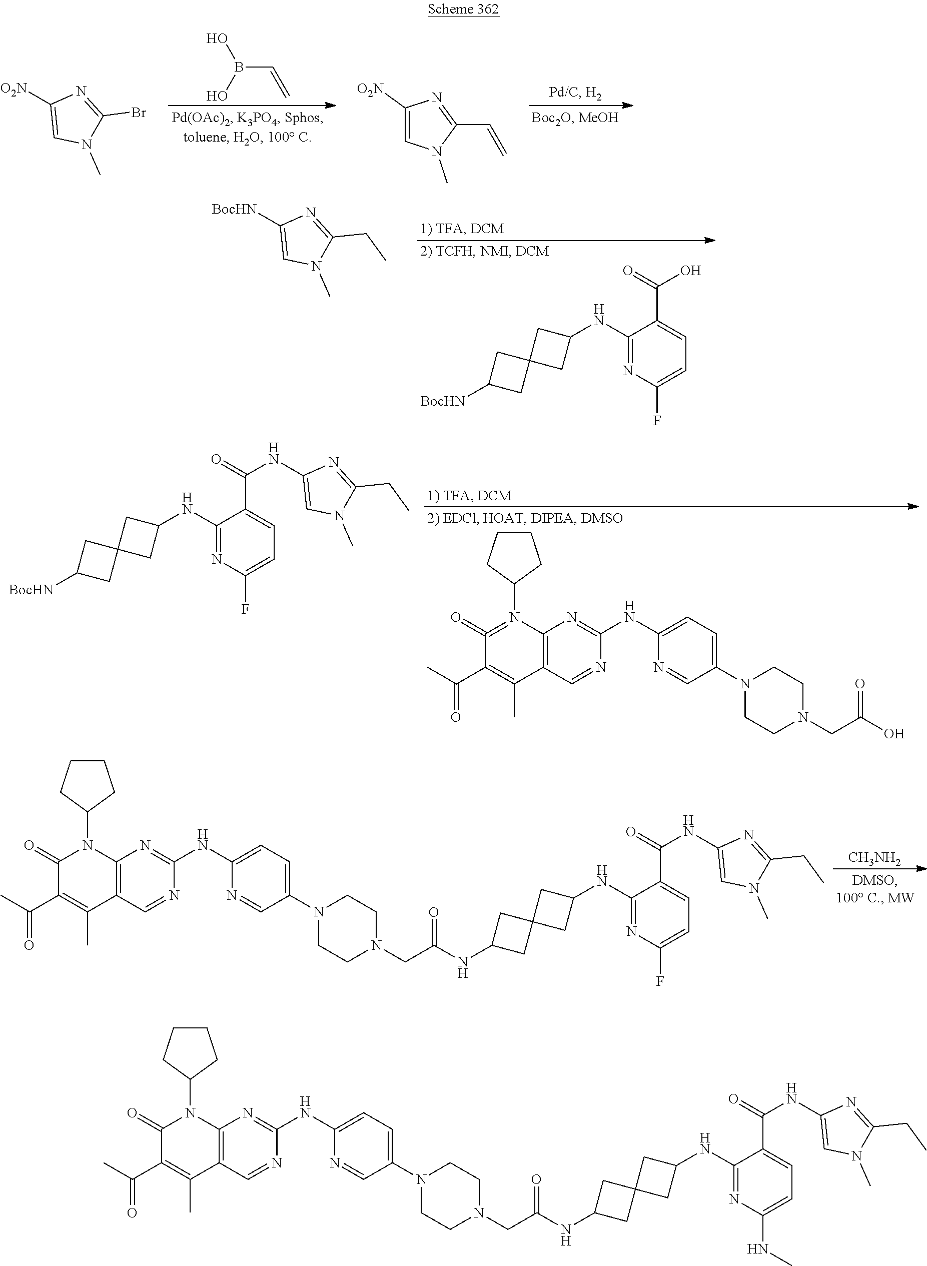

CPD-308 was synthesized following the standard procedures for preparing CPD-296 (12.1 mg, 7% yield for 7 steps) as a yellow solid. MS (ESI) m/z=871.7 [M+H]⁺.

### Example 363. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-methyl-2-propyl-1H-imidazol-4-yl)-6-(methylamino)nicotinamide (CPD-309)

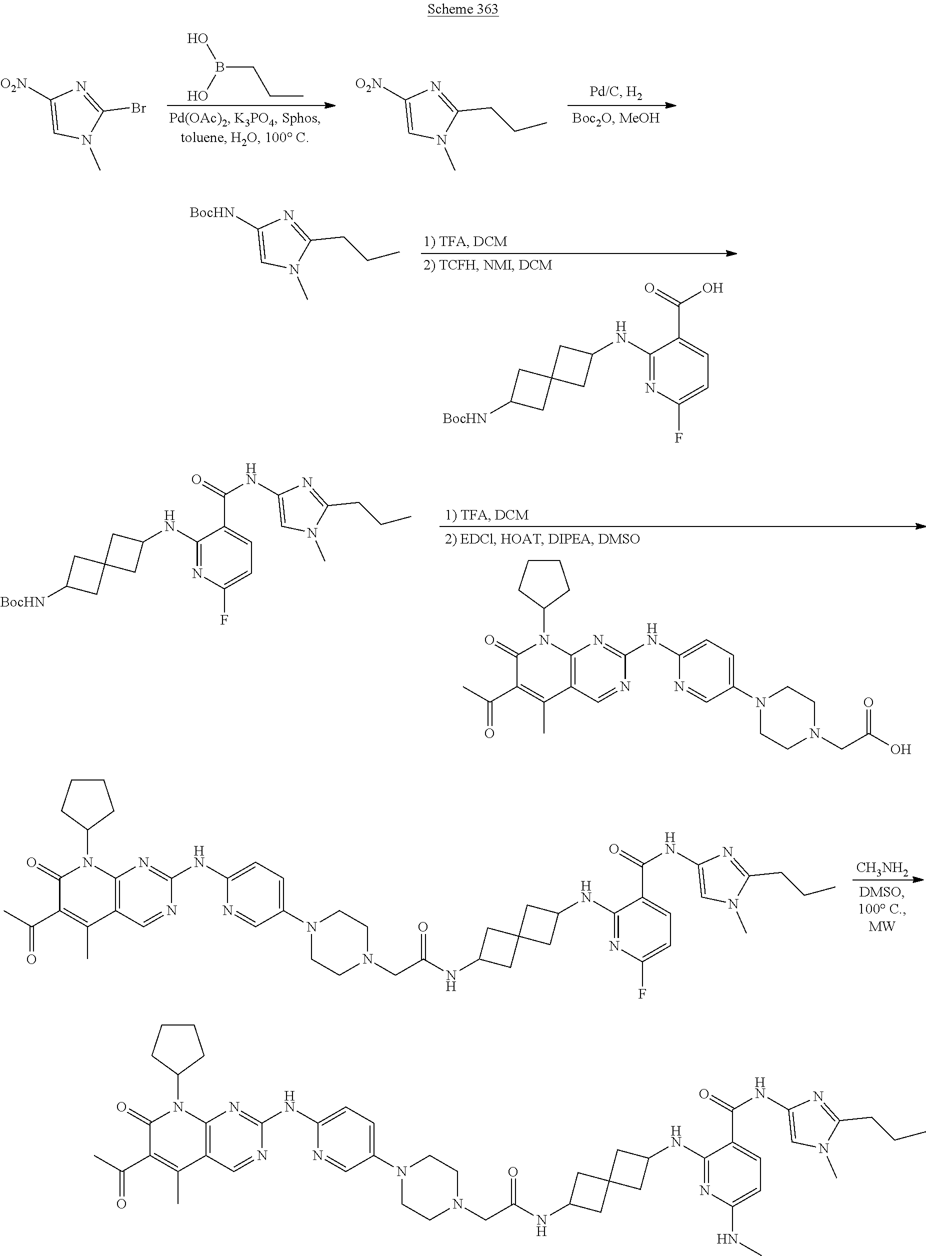

CPD-309 was synthesized following the standard procedures for preparing CPD-296 (8.4 mg, 3% yield for 7 steps) as a yellow solid. MS (ESI) m/z=885.7 [M+H]⁺.

### Example 364. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclobutyl-1-methyl-1H-imidazol-4-yl)-6-(methylamino)nicotinamide (CPD-310)

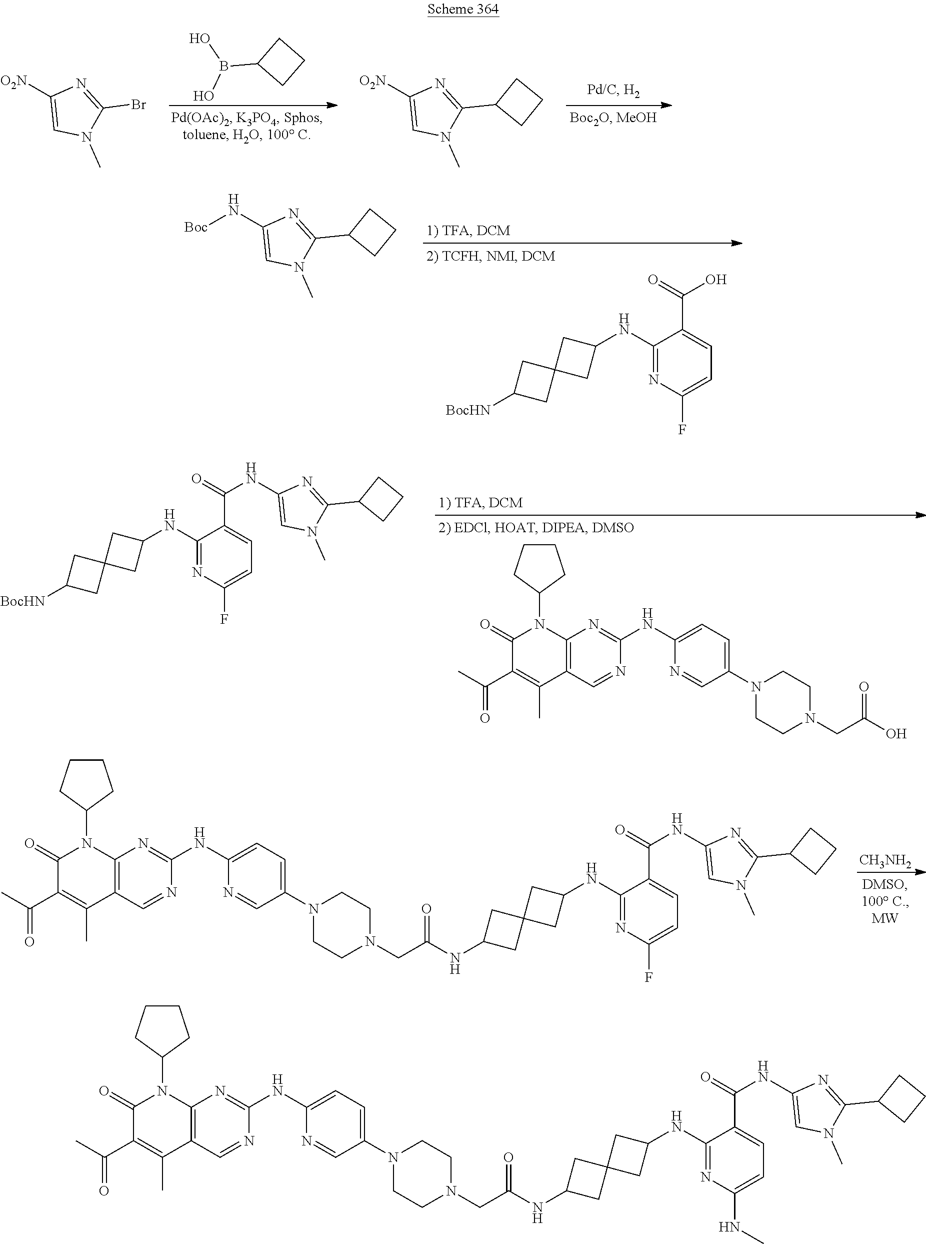

CPD-310 was synthesized following the standard procedures for preparing CPD-296 (7.1 mg, 3% yield for 7 steps) as a yellow solid. MS (ESI) m/z=897.7 [M+H]⁺.

### Example 365. 3-((3-((6-(dimethylamino)pyridazin-3-yl)carbamoyl)-6-(methylamino)pyridin-2-yl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (CPD-311)

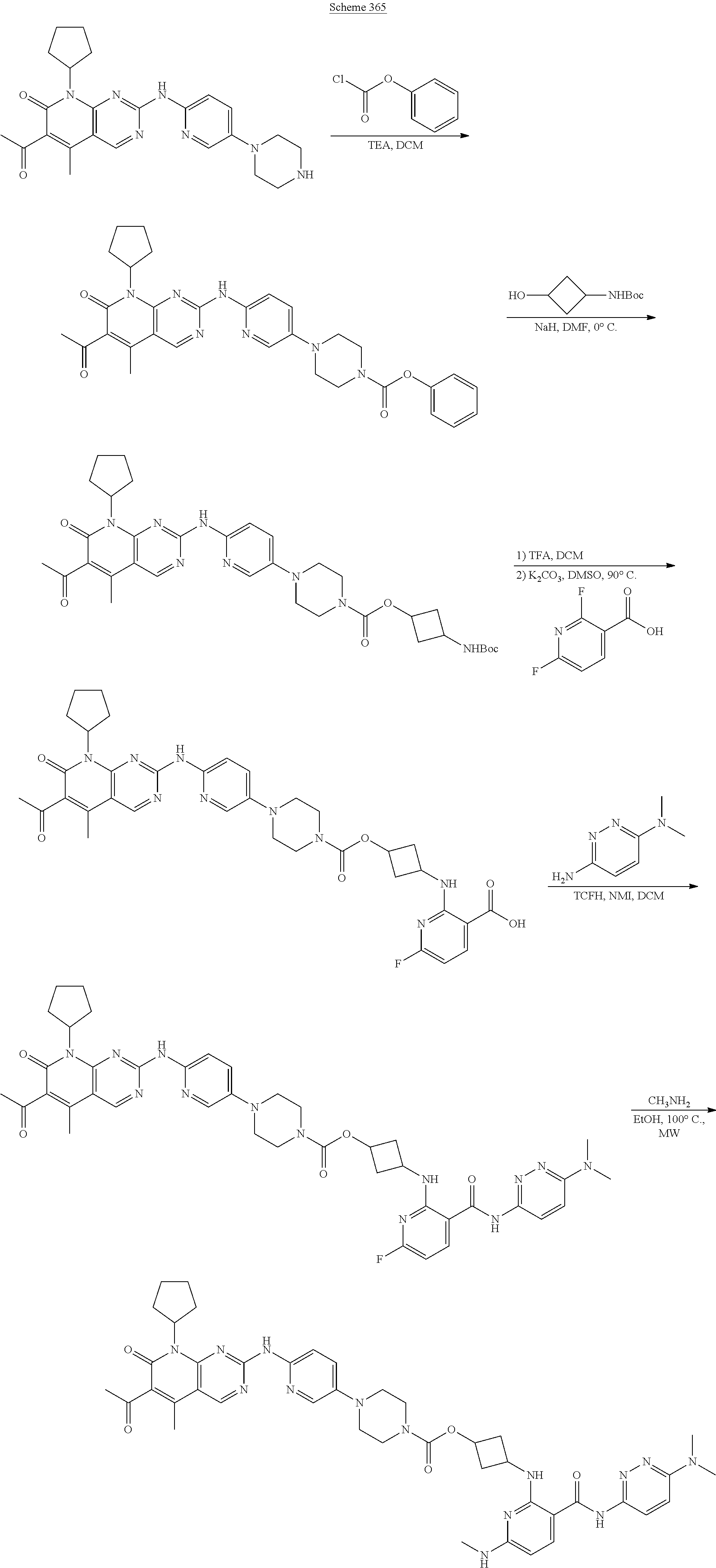

### Step 1. Synthesis of phenyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

A mixture of 6-acetyl-8-cyclopentyl-5-methyl-2-((5-(piperazin-1-yl)pyridin-2-yl)amino)pyrido[2,3-d]pyrimidin-7(8H)-one (208 mg, 0.46 mmol), phenyl carbonochloridate (73 mg, 0.46 mmol) and TEA (141 mg, 1.39 mmol) in dichloromethane (10 mL) was stirred at rt for 16 h. The reaction mixture was poured into water (10 mL) and extracted with dichloromethane (3×10 mL). The combined organic layers were washed with saturated brine (10 mL), dried over anhydrous Na₂SO₄, filtered and concentrated. The resulting residue was purified by silica gel flash chromatography to give the desired product (162 mg, 61%~yield) as a red solid. MS (ESI) m/z=568.5 [M+H]⁺.

### Step 2. Synthesis of 3-((tert-butoxycarbonyl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

To a solution of tert-butyl (3-hydroxycyclobutyl)carbamate (50 mg, 0.26 mmol) in DMF (5 mL) was added sodium hydride (32 mg, 0.8 mmol, 60 wt % in mineral oil) at 0° C. After stirring at 0° C. for 0.5 h, phenyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (151 mg, 0.26 mmol) in DMF (2 mL) was added [symbol] dropwise at 0° C. The reaction mixture was stirred at rt for 16 h. The mixture was quenched with water (10 mL) slowly and extracted with ethyl acetate (3×10 mL). The combined organic layers were washed with brine, dried over anhydrous Na₂SO₄, filtered and concentrated. The residue was purified by reverse-phase chromatography to give the desired product (76 mg, 36% yield) as a yellow solid. MS (ESI) m/z=661.6 [M+H]⁺.

### Step 3. Synthesis of 3-aminocyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

To a solution of 3-((tert-butoxycarbonyl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (76 mg, 0.09 mmol) in dichloromethane (2 mL) was added TFA (1 mL). The reaction mixture was stirred at rt for 1 h. The mixture was concentrated to give the crude product (66 mg, 99% yield) as a yellow oil. This compound was used in the next step directly without further purification. MS (ESI) m/z=561.5 [M+H]⁺.

### Step 4. Synthesis of 2-((3-((4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)oxy)cyclobutyl)amino)-6-fluoronicotinic acid

To a solution of 3-aminocyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (66 mg, 0.09 mmol) in DMSO (5 mL) were added 2,6-difluoronicotinic acid (18 mg, 0.11 mmol) and K₂CO₃ (42 mg, 0.29 mmol). The reaction mixture was stirred 90° C. for 16 h. The reaction mixture was purified by reverse-phase chromatography to give the desired product (55 mg, 69% yield) as a yellow solid. MS (ESI) m/z=700.5 [M+H]⁺.

### Step 5. Synthesis of 3-((3-((6-(dimethylamino)pyridazin-3-yl)carbamoyl)-6-fluoropyridin-2-yl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

A mixture of 2-((3-((4-(6-((6-acetyl-8-cyclopentyl-5-methyl-7-oxo-7,8-dihydropyrido[2,3-d]pyrimidin-2-yl)amino)pyridin-3-yl)piperazine-1-carbonyl)oxy)cyclobutyl)amino)-6-fluoronicotinic acid (54 mg, 0.07 mmol), N,N′-dimethylpyridazine-3,6-diamine (11 mg, 0.07 mmol), TCFH (43 mg, 0.15 mmol) and NMI (32 mg, 0.38 mmol) in dichloromethane (5 mL) was stirred at rt for 2 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by silica gel flash chromatography to give the desired product (35 mg, 33% yield) as a colorless oil. MS (ESI) m/z=820.5 [M+H]⁺.

### Step 6. Synthesis of 3-((3-((6-(dimethylamino)pyridazin-3-yl)carbamoyl)-6-(methylamino)pyridin-2-yl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate

To a solution of 3-((3-((6-(dimethylamino)pyridazin-3-yl)carbamoyl)-6-fluoropyridin-2-yl)amino)cyclobutyl 4-(6-((6-acetyl-8-cyclopentyl-5-methyl-7-oxo-7,8-dihydropyrido[2,3-d]pyrimidin-2-yl)amino)pyridin-3-yl)piperazine-1-carboxylate (35 mg, 0.04 mmol) in EtOH (1 mL) was added methanamine (30% in EtOH, 1 mL). The reaction mixture was stirred at 100° C. for 1 h under microwave irradiation. The reaction mixture was purified by reverse-phase chromatography to give the desired product (5.3 mg, 13% yield) as a white solid. MS (ESI) m/z=831.6 [M+H]⁺.

### Example 366. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-ethyl-5-methyl-1H-pyrazol-3-yl)-6-(methylamino)nicotinamide (CPD-312)

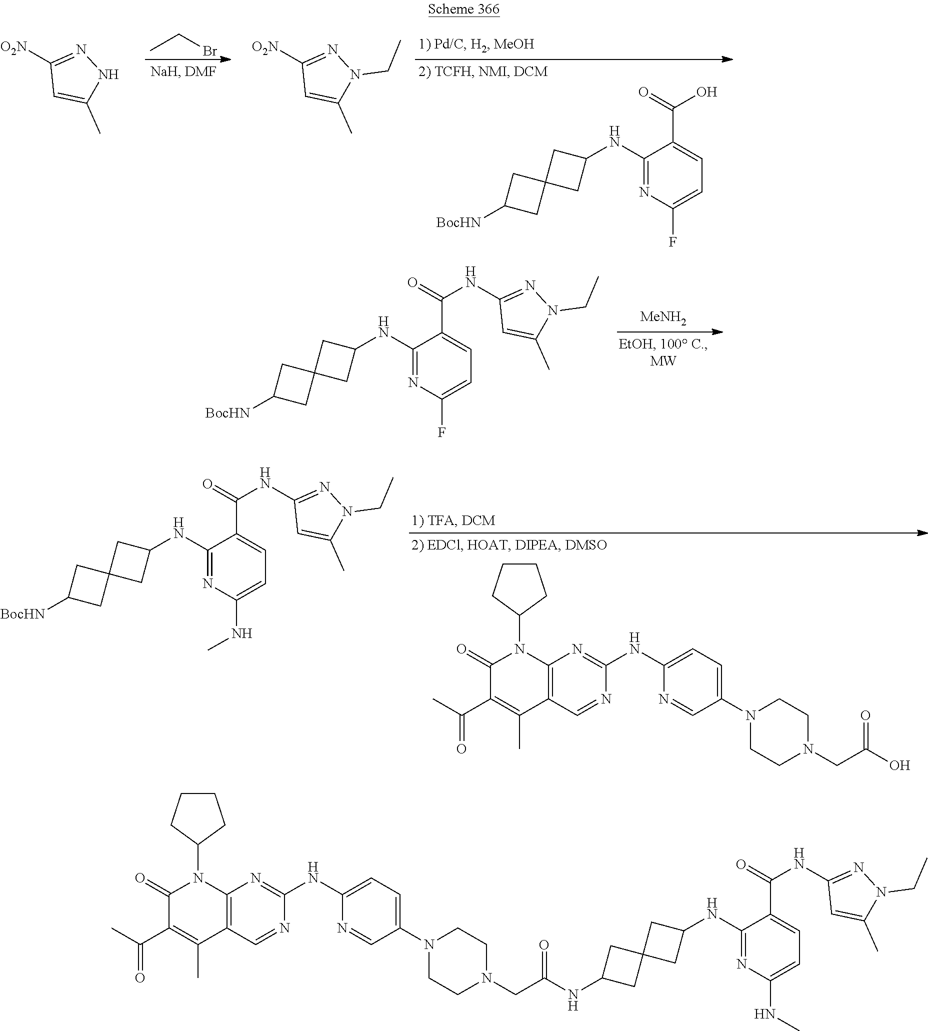

### Step 1. Synthesis of 1-ethyl-5-methyl-3-nitro-1H-pyrazole

To a solution of 5-methyl-3-nitro-1H-pyrazole (500 mg, 3.93 mmol) in DMF (10 mL) was added sodium hydride (226 mg, 5.9 mmol, 60 wt % in mineral oil) in portions at 0° C. After stirring at 0° C. for 0.5 h, bromoethane (643 mg, 5.9 mmol) in DMF (2 mL) was added dropwise at 0 [symbol] . The reaction mixture was stirred at rt for 16 h. The reaction mixture was quenched with water (10 mL) slowly and extracted with ethyl acetate (3×10 mL). The combined organic phase was washed with brine, dried over anhydrous Na₂SO₄, filtered and concentrated. The residue was purified by silica gel flash chromatography to give the desired product (326 mg, 53% yield) as a yellow solid. MS (ESI) m/z=156.1 [M+H]⁺.

### Step 2. Synthesis of 1-ethyl-5-methyl-1H-pyrazol-3-amine

To a solution of 1-ethyl-5-methyl-3-nitro-1H-pyrazole (50 mg, 0.32 mmol) in MeOH (5 mL) was added 10% Pd/C (10 mg). The reaction mixture was stirred at rt for 3 h under hydrogen balloon. The mixture was filtered and concentrated to give the desired product (32 mg, 79% yield) as a colorless oil. MS (ESI) m/z=126.1 [M+H]⁺.

### Step 3. Synthesis of tert-butyl (6-((3-((1-ethyl-5-methyl-1H-pyrazol-3-yl)carbamoyl)-6-fluoropyridin-2-yl)amino)spiro[3.3]heptan-2-yl)carbamate

A mixture of 2-((6-((tert-butoxycarbonyl)amino)spiro[3.3]heptan-2-yl)amino)-6-fluoronicotinic acid (40 mg, 0.11 mmol), 1-ethyl-5-methyl-1H-pyrazol-3-amine (27 mg, 0.22 mmol), TCFH (61 mg, 0.22 mmol) and NMI (45 mg, 0.54 mmol) in dichloromethane (2 mL) was stirred at rt for 2 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by silica gel flash chromatography to give the desired product (35 mg, 67% yield) as a colorless oil. MS (ESI) m/z=473.5 [M+H]⁺.

### Step 4. Synthesis of tert-butyl (6-((3-((1-ethyl-5-methyl-1H-pyrazol-3-yl)carbamoyl)-6-(methylamino)pyridin-2-yl)amino)spiro[3.3]heptan-2-yl)carbamate

To a solution of tert-butyl (6-((3-((1-ethyl-5-methyl-1H-pyrazol-3-yl)carbamoyl)-6-fluoropyridin-2-yl)amino)spiro[3.3]heptan-2-yl)carbamate (35 mg, 0.07 mmol) in EtOH (1 mL) was added methanamine (30% in EtOH, 1 mL). The reaction mixture was stirred at 100° C. for 1 h under microwave irradiation. The reaction mixture was purified by silica gel flash chromatography to give the desired product (27 mg, 75% yield) as a white solid. MS (ESI) m/z=484.4 [M+H]⁺.

### Step 5. Synthesis of 2-((6-aminospiro[3.3]heptan-2-yl)amino)-N-(1-ethyl-5-methyl-1H-pyrazol-3-yl)-6-(methylamino)nicotinamide

To a solution of tert-butyl (6-((3-((1-ethyl-5-methyl-1H-pyrazol-3-yl)carbamoyl)-6-(methylamino)pyridin-2-yl)amino)spiro[3.3]heptan-2-yl)carbamate (25 mg, 0.05 mmol) in dichloromethane (1 mL) was added TFA (1 mL). The reaction mixture was stirred at rt for 1 h. The reaction mixture was concentrated to give the desired product (25 mg, 97% yield) as a yellow oil. This compound was used directly in the next step without further purification. MS (ESI) m/z=384.4 [M+H]⁺.

### Step 6. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-ethyl-5-methyl-1H-pyrazol-3-yl)-6-(methylamino)nicotinamide

A mixture of 2-((6-aminospiro[3.3]heptan-2-yl)amino)-N-(1-ethyl-5-methyl-1H-pyrazol-3-yl)-6-(methylamino)nicotinamide (25 mg, 0.05 mmol), 5-((3-((6-(dimethylamino)pyridazin-3-yl)carbamoyl)-6-(methylamino)pyridin-2-yl)amino)pentanoic acid (25 mg, 0.05 mmol), EDCI (15 mg, 0.07 mmol), HOAT (11 mg, 0.07 mmol) and DIPEA (32 mg, 0.25 mmol) in DMSO (2 mL) was stirred at rt for 16 h. The reaction mixture was purified by reverse-phase chromatography to give the desired product (21.6 mg, 43% yield) as a yellow solid. MS (ESI) m/z=871.5 [M+H]⁺.

### Example 367. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)piperidin-4-yl)amino)-N-(2-cyclopropyl-1-methyl-1H-imidazol-4-yl)-6-(isopropylamino)nicotinamide (CPD-313)

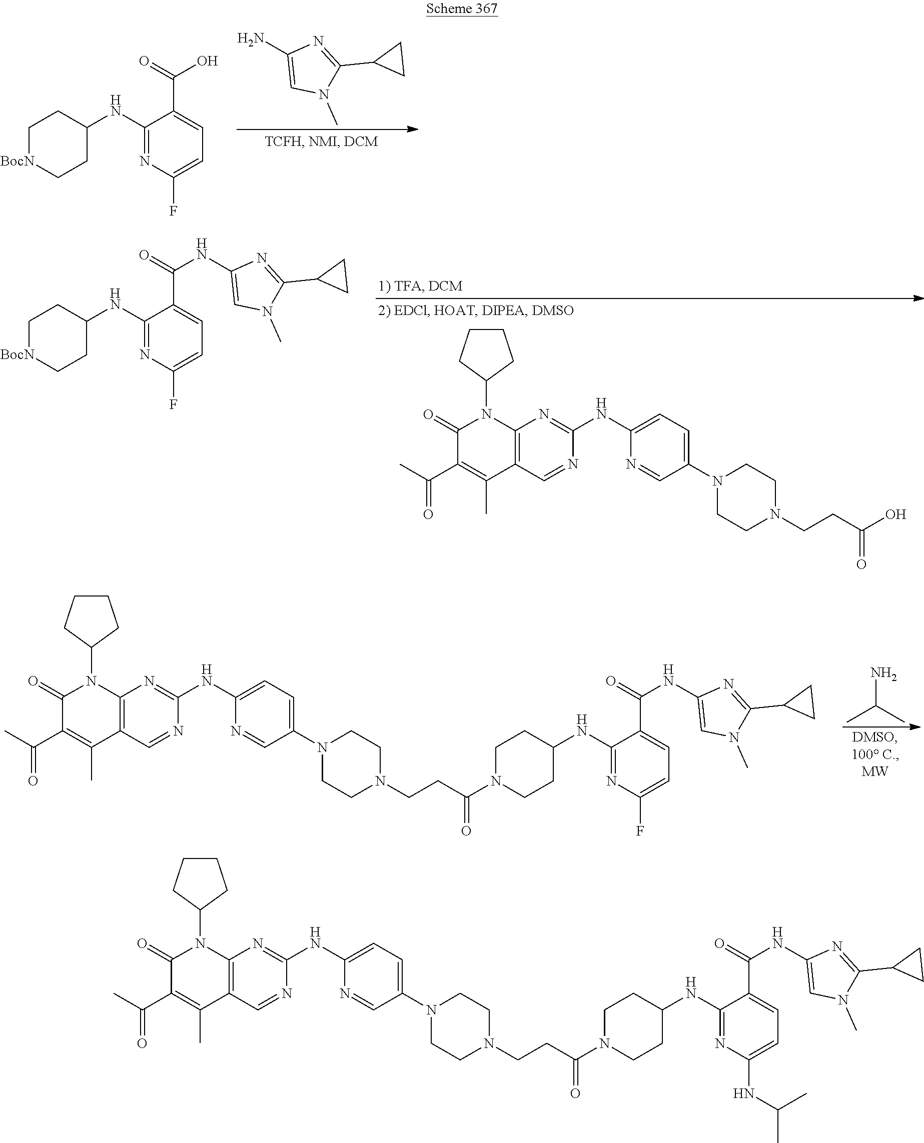

CPD-313 was synthesized following the standard procedures for preparing CPD-144 and CPD-253 (11.8 mg, 24% yield for 4 steps) as a yellow solid. MS (ESI) m/z=899.6 [M+H]⁺.

### Example 368. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5-methyl-1-propyl-1H-pyrazol-3-yl)-6-(methylamino)nicotinamide (CPD-314)

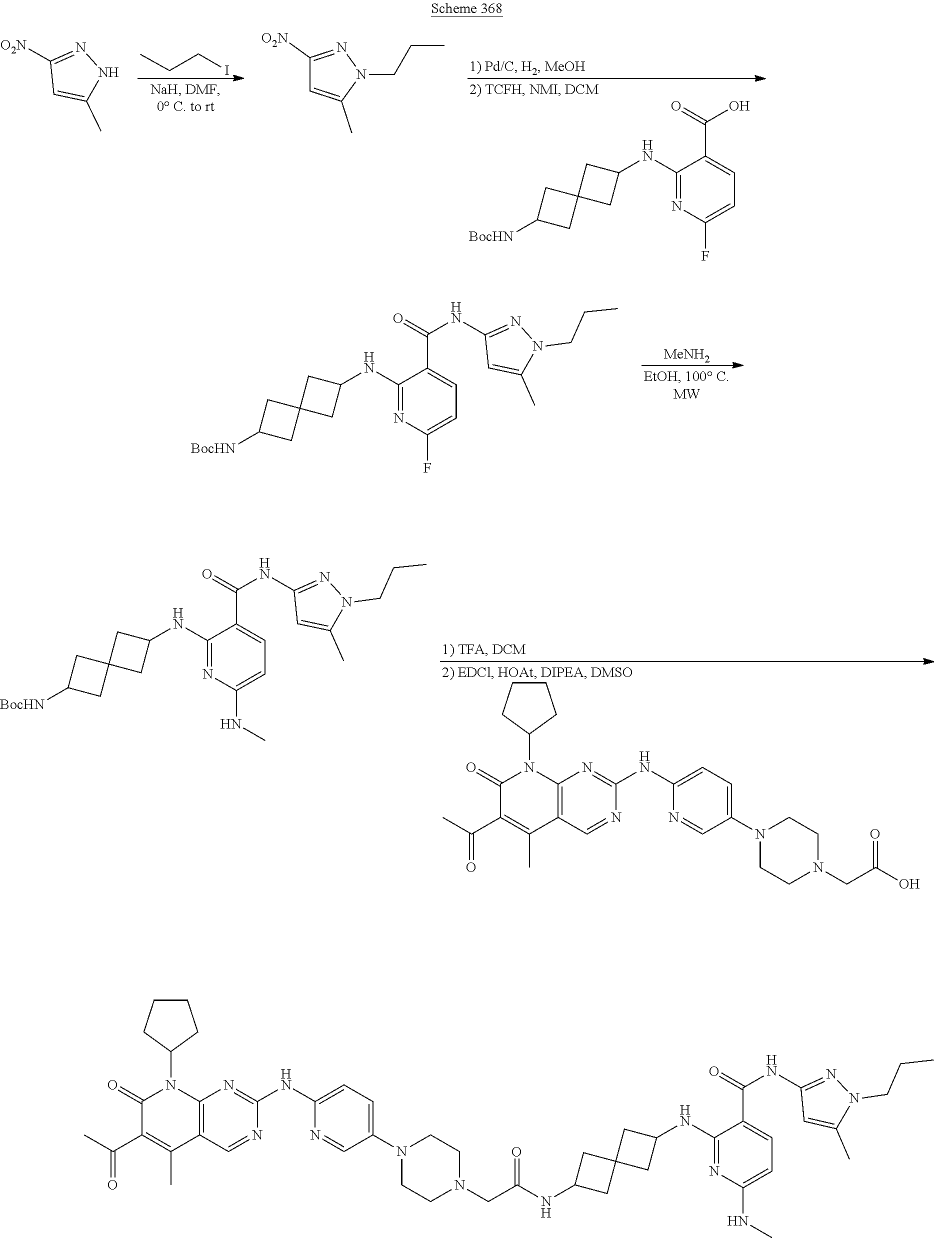

CPD-314 was synthesized following the standard procedures for preparing CPD-312 (21.7 mg, 14% yield for 6 steps) as a yellow solid. MS (ESI) m/z=885.6 [M+H]⁺.

### Example 369. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(1-cyclopropyl-5-methyl-1H-pyrazol-3-yl)-6-(methylamino)nicotinamide (CPD-315)

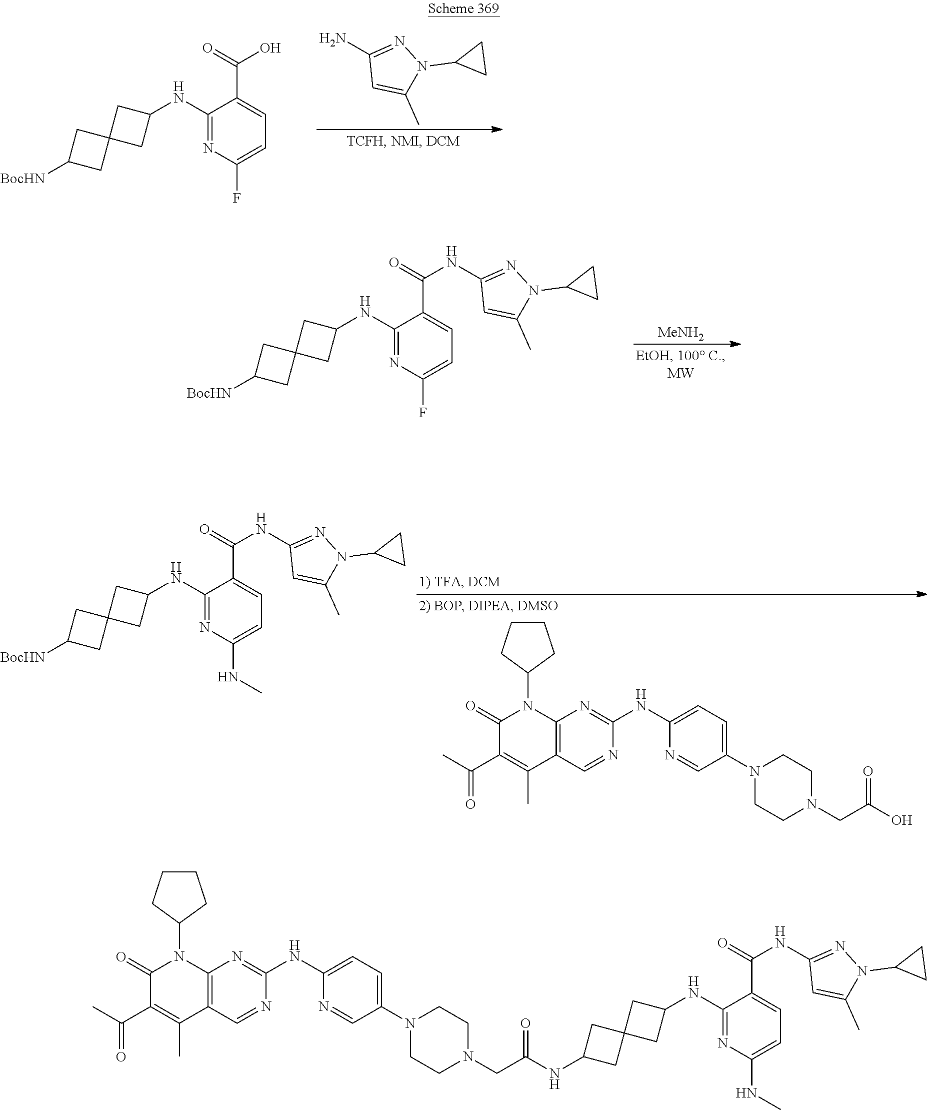

CPD-315 was synthesized following the standard procedures for preparing CPD-144 (11.0 mg, 18% yield for 4 steps) as a yellow solid. MS (ESI) m/z=883.5 [M+H]⁺.

### Example 370. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-(isopropylamino)nicotinamide (CPD-316)

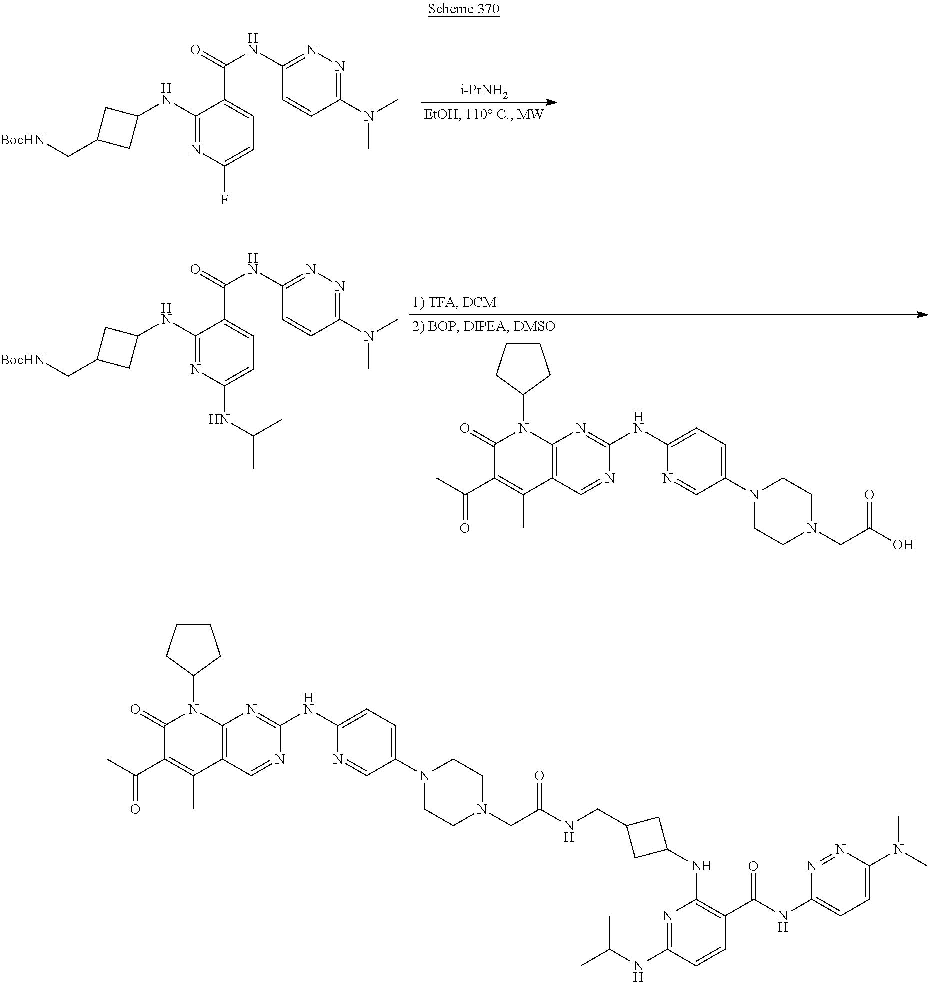 [SMILES: "i-PrNH2" ; CN(C)c1ccc(NC(=O)c2ccc(F)nc2NC2CC(CNC(=O)OC(C)(C)C)C2)nn1 ; CC(C)Nc1ccc(C(=O)Nc2ccc(N(C)C)nn2)c(NC2CC(CNC(=O)OC(C)(C)C)C2)n1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NCC5CC(Nc6nc(NC(C)C)ccc6C(=O)Nc6ccc(N(C)C)nn6)C5)CC4)cn3)nc2n(C2CCCC2)c1=O]

CPD-316 was synthesized following the standard procedures for preparing CPD-144 (8.0 mg, 12% yield for 3 steps) as a yellow solid. MS (ESI) m/z=886.6 [M+H]⁺.

### Example 371. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)oxy)-N-(2-cyclopropyl-1-methyl-1H-imidazol-4-yl)-6-(isopropylamino)nicotinamide (CPD-317)

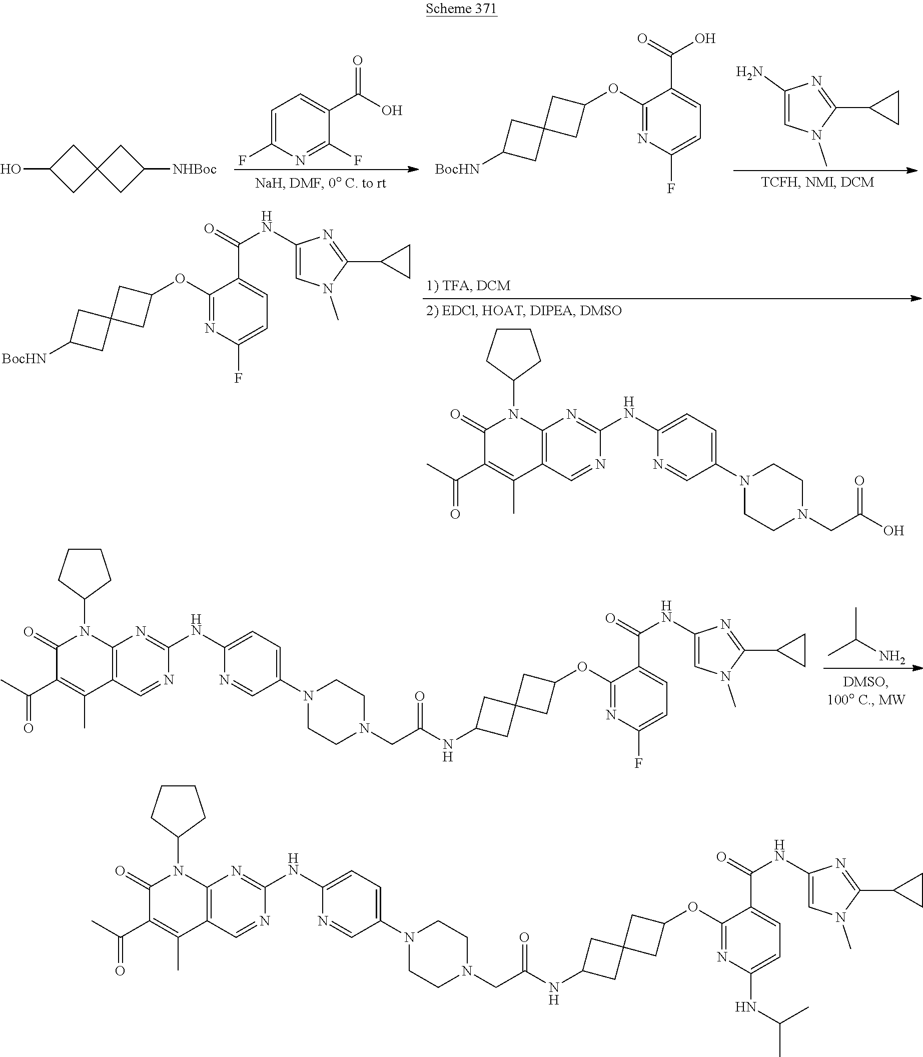

### Step 1. Synthesis of 2-((6-((tert-butoxycarbonyl)amino)spiro[3.3]heptan-2-yl)oxy)-6-fluoronicotinic acid

To a solution of tert-butyl (6-hydroxyspiro[3.3]heptan-2-yl)carbamate (50 mg, 0.22 mmol) in DMF (5 mL) was added sodium hydride (16 mg, 0.65 mmol, 60% in mineral oil) in portions at 0° C. After stirring at 0° C. for 0.5 h, 2,6-difluoronicotinic acid (35 mg, 0.22 mmol) in DMF (2 mL) was added dropwise at 0° C. The reaction mixture was stirred at rt for 16 h. The mixture was purified by reverse-phase chromatography to give the desired product (35 mg, 33% yield) as a white solid. MS (ESI) m/z=267.2 [M-100]⁺.

The remaining steps were performed according to the procedures for preparing CPD-144 and CPD-253 to provide the desired product (3.1 mg, 10% yield over 4 steps) as a white solid. MS (ESI) m/z=912.7 [M+H]⁺.

### Example 372. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-isobutylnicotinamide (CPD-318)

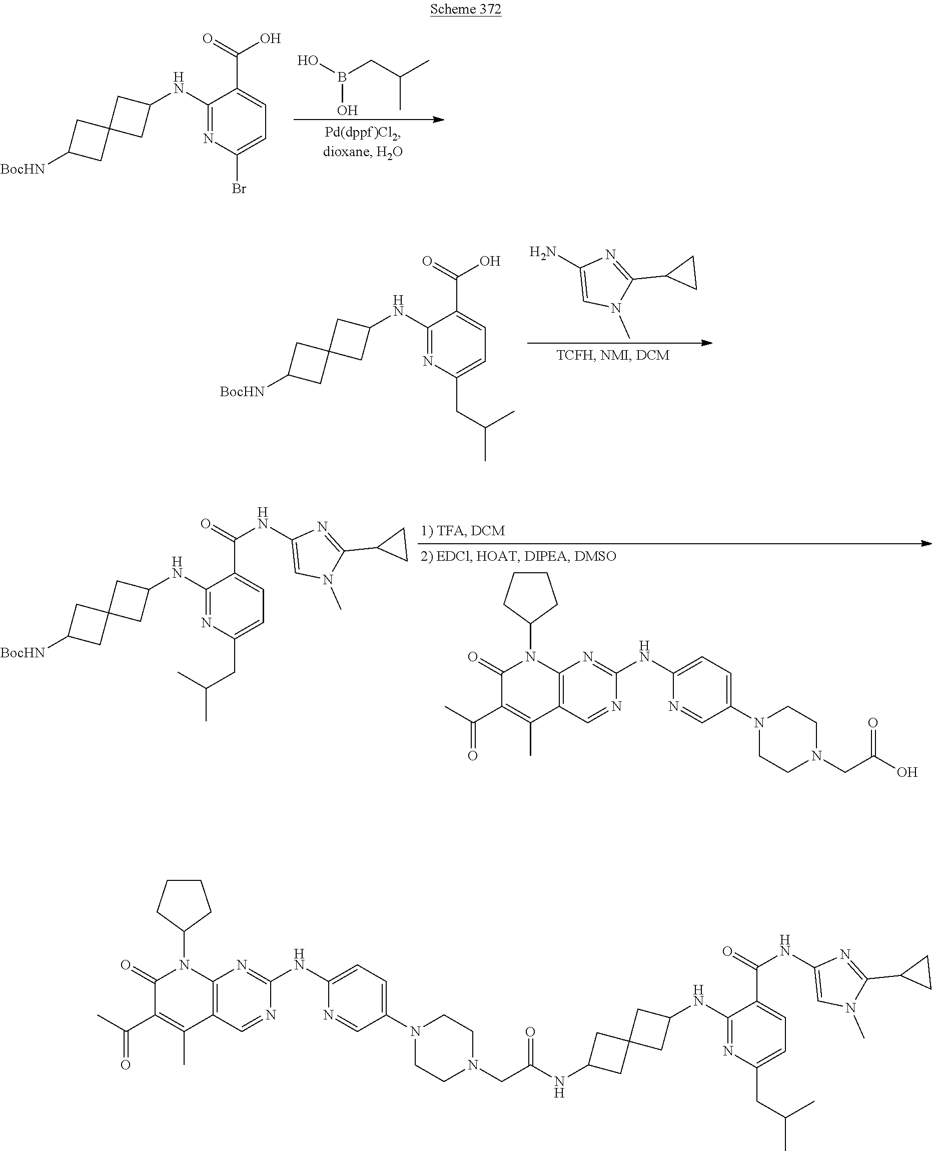

### Step 1. Synthesis of 2-((6-((tert-butoxycarbonyl)amino)spiro[3.3]heptan-2-yl)amino)-6-isobutylnicotinic acid

To a mixture of 6-bromo-2-((6-((tert-butoxycarbonyl)amino)spiro[3.3]heptan-2-yl)amino)nicotinic acid (130 mg, 0.31 mmol) and isobutylboronic acid (155 mg, 1.52 mmol) in dioxane (5 mL) and H₂O (1 mL) were added K₂CO₃ (126 mg, 0.9 mmol) and Pd(dppf)Cl₂ (23 mg, 0.03 mmol) at room temperature. The reaction mixture was stirred at 100° C. for 5 h under N₂. The mixture was concentrated and purified by reverse phase chromatography to give the desired product (45 mg, 13% yield) as a brown oil. MS (ESI) m/z=404.6 [M+H]⁺.

The remaining steps were performed according to the procedures for preparing CPD-144 to provide the desired product (11.1 mg, 5% yield over 3 steps) as a yellow solid. MS (ESI) m/z 910.7 [M+H]⁺.

### Example 373. 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-N-(6-(dimethylamino)pyridazin-3-yl)-6-(methylamino)nicotinamide (CPD-319)

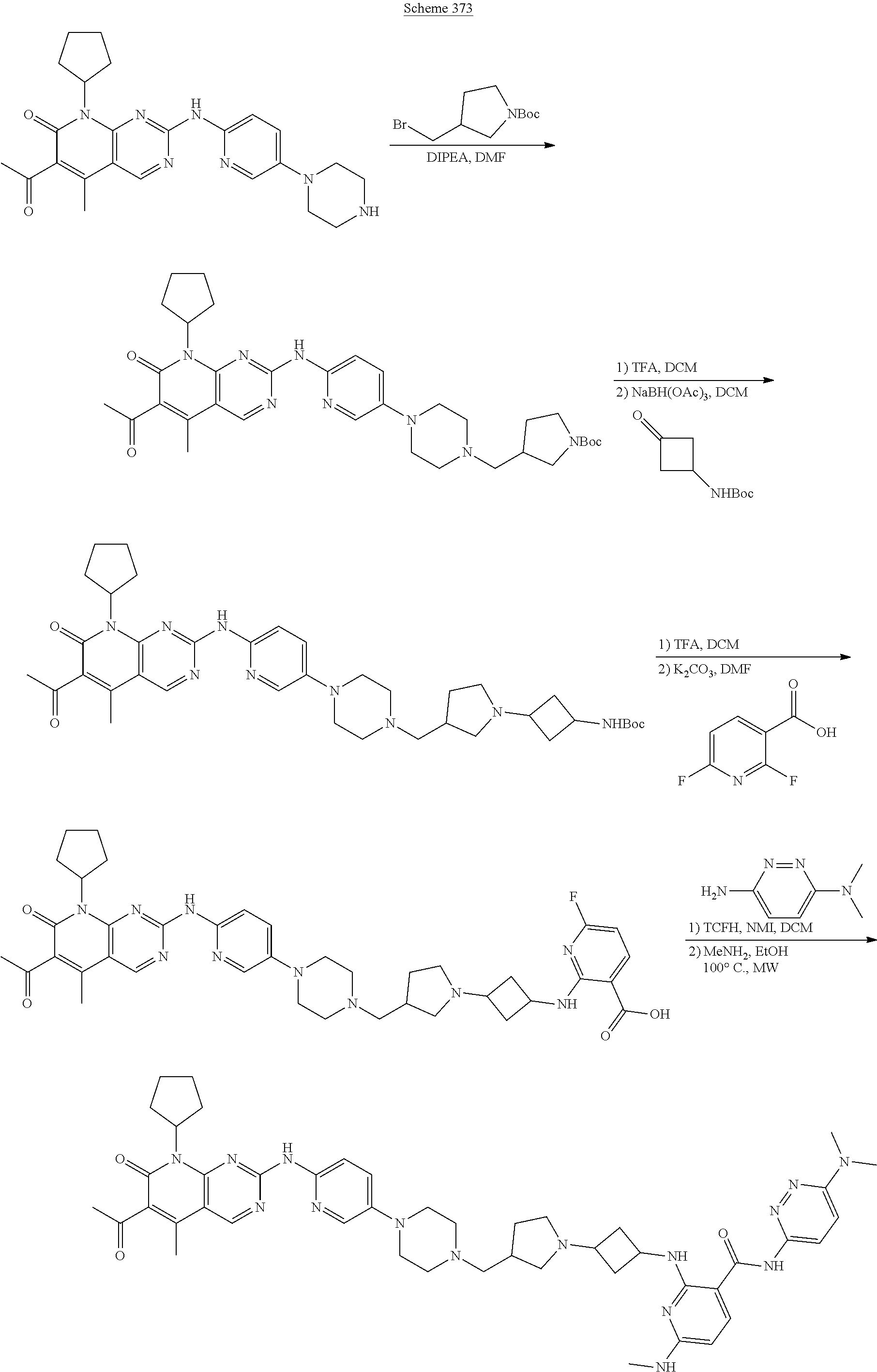

### Step 1. Synthesis of tert-butyl 3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidine-1-carboxylate

To a solution of 6-acetyl-8-cyclopentyl-5-methyl-2-((5-(piperazin-1-yl)pyridin-2-yl)amino)pyrido[2,3-d]pyrimidin-7(8H)-one (100 mg, 0.22 mmol) in DMF (5 mL) were added tert-butyl 3-(bromomethyl)pyrrolidine-1-carboxylate (59 mg, 0.22 mmol) and DIPEA (289 mg, 2.22 mmol) at rt. The reaction mixture was stirred at 90° C. for 16 h. The mixture was cooled to rt. The mixture was diluted with water (30 mL) and extracted with dichloromethane (3×30 mL). The combined organic phase was washed with brine, dried over Na₂SO₄, filtered, and concentrated to give the crude. The residue was purified by reverse-phase chromatography to provide the desired product (90 mg, 64% yield) as a light-yellow solid. MS (ESI) m/z=631.5 [M+H]⁺.

### Step 2. Synthesis of 6-acetyl-8-cyclopentyl-5-methyl-2-((5-(4-(pyrrolidin-3-ylmethyl)piperazin-1-yl)pyridin-2-yl)amino)pyrido[2,3-d]pyrimidin-7(8H)-one

To a solution of tert-butyl 3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidine-1-carboxylate (90 mg, 0.14 mmol) in dichloromethane (5 mL) was added TFA (2 mL). The mixture was stirred at rt for 1 h. Then the solution was concentrated under reduced pressure to provide the crude product (70 mg, 92% yield) as a light-yellow oil. This compound was used directly in the next step without further purification. MS (ESI) m/z=531.4 [M+H]⁺.

### Step 3. Synthesis of tert-butyl (3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)carbamate

To a solution of 6-acetyl-8-cyclopentyl-5-methyl-2-((5-(4-(pyrrolidin-3-ylmethyl)piperazin-1-yl)pyridin-2-yl)amino)pyrido[2,3-d]pyrimidin-7(8H)-one (70 mg, 0.13 mmol) in dichloromethane (10 mL) were added sodium triacetoxyborohydride (80 mg, 0.39 mmol) and tert-butyl (3-oxocyclobutyl)carbamate [symbol] (120 mg, 0.65 mmol) at rt. Then the reaction mixture was stirred at 45 for 3 h. After cooling down to rt, the reaction mixture was poured into water (50 mL) and extracted with ethyl acetate (3×50 mL). The combined organic layers were washed with saturated brine (30 mL), dried over anhydrous sodium sulfate, filtered, and evaporated under reduced pressure. The resulting residue was purified by reverse-phase chromatography to provide the desired product (60 mg, 65% yield) as a light-yellow solid. MS ESI) m/z=700.6 [M+H]⁺.

### Step 4. Synthesis of 6-acetyl-2-((5-(4-((1-(3-aminocyclobutyl)pyrrolidin-3-yl)methyl)piperazin-1-yl)pyridin-2-yl)amino)-8-cyclopentyl-5-methylpyrido[2,3-d]pyrimidin-7(8H)-one

To a solution of tert-butyl (3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)carbamate (60 mg, 0.08 mmol) in dichloromethane (5 mL) was added TFA (2 mL). The mixture was stirred at rt for 1 h. Then the solution was concentrated under reduced pressure to provide the crude product (50 mg, 97% yield) as a yellow oil. This compound was used directly in the next step without further purification. MS (ESI) m/z=600.6 [M+H]⁺.

### Step 5. Synthesis of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-6-fluoronicotinic acid

To a solution of 6-acetyl-2-((5-(4-((1-(3-aminocyclobutyl)pyrrolidin-3-yl)methyl)piperazin-1-yl)pyridin-2-yl)amino)-8-cyclopentyl-5-methylpyrido[2,3-d]pyrimidin-7(8H)-one (50 mg, 0.08 mmol) in DMF (5 mL) were added 2,6-difluoropyridine-3-carboxylic acid (13 mg, 0.08 mmol) and K₂CO₃ (11 mg, 0.08 mmol) at rt. The reaction mixture was stirred at 90° C. for 16 h. The mixture was cooled to rt. The mixture was diluted with water (20 mL) and extracted with dichloromethane (3×20 mL). The combined organic phase was washed with brine, dried over Na₂SO₄, filtered and concentrated. The residue was purified by reverse-phase chromatography to provide the desired product (20 mg, 32% yield) as a yellow solid. MS (ESI) m/z=739.5 [M+H]⁺.

### Step 6. Synthesis of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-N-(6-(dimethylamino)pyridazin-3-yl)-6-fluoronicotinamide

To a mixture of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-6-fluoronicotinic acid (20 mg, 0.03 mmol) and NMI (7 mg, 0.09 mmol) in dichloromethane (5 mL) were added N³,N³-dimethylpyridazine-3,6-diamine (4 mg, 0.03 mmol)) and TCFH (16 mg, 0.06 mmol). The mixture was stirred at rt for 2 h. The mixture was quenched with water (5 mL) and extracted with dichloromethane (3×10 mL). The combined organic phase was washed with brine, dried over Na₂SO₄, filtered, and concentrated to give the crude. The residue was purified by silica gel chromatography to provide the desired product (6 mg, 25% yield) as a light-yellow solid. MS (ESI) m/z=859.7 [M+H]⁺.

### Step 7. Synthesis of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-N-(6-(dimethylamino)pyridazin-3-yl)-6-(methylamino)nicotinamide

To a solution of 2-((3-(3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)cyclobutyl)amino)-N-(6-(dimethylamino)pyridazin-3-yl)-6-fluoronicotinamide (6 mg, 7 mol) in ethanol (0.5 mL) was added CH₃NH₂ solution (30% in ethanol, 0.5 mL). The reaction mixture was stirred at 100° C. under microwave irradiation for 1 h. After cooling down to rt, the reaction mixture was concentrated and purified by reverse-phase chromatography to provide the desired product (1.5 mg, 24% yield) as a yellow solid. MS (ESI) m/z=870.5 [M+H]⁺.

### Example 374. 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(2-cyclopropyl-1-methyl-1H-imidazol-4-yl)-6-(methylamino)nicotinamide (CPD-320)

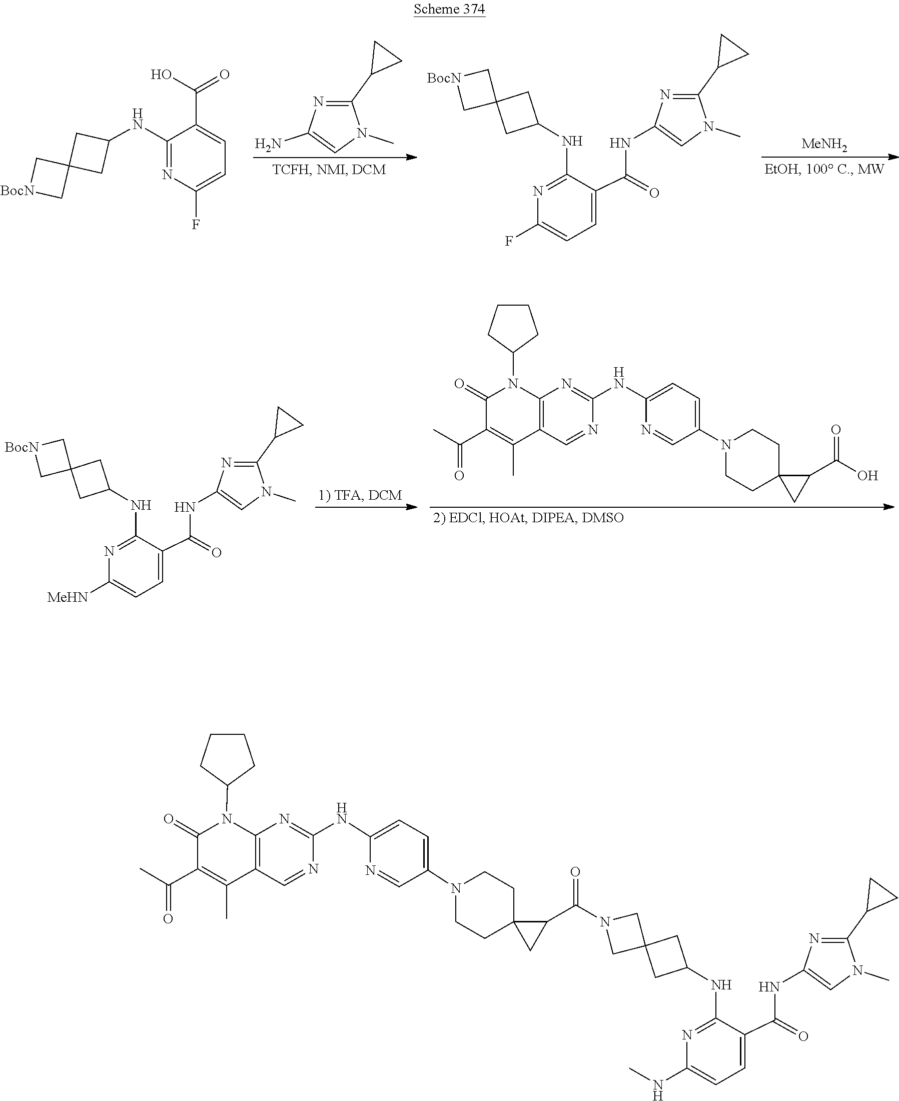

CPD-320 was synthesized following the standard procedures for preparing CPD-144 (3.0 mg, 11% yield for 4 steps) as a yellow solid. MS (ESI) m/z=880.6 [M+H]⁺.

### Example 375. 2-((2-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)-2-azaspiro[3.3]heptan-6-yl)amino)-N-(2-cyclopropyl-1-methyl-1H-imidazol-4-yl)-6-(methylamino)nicotinamide (CPD-321)

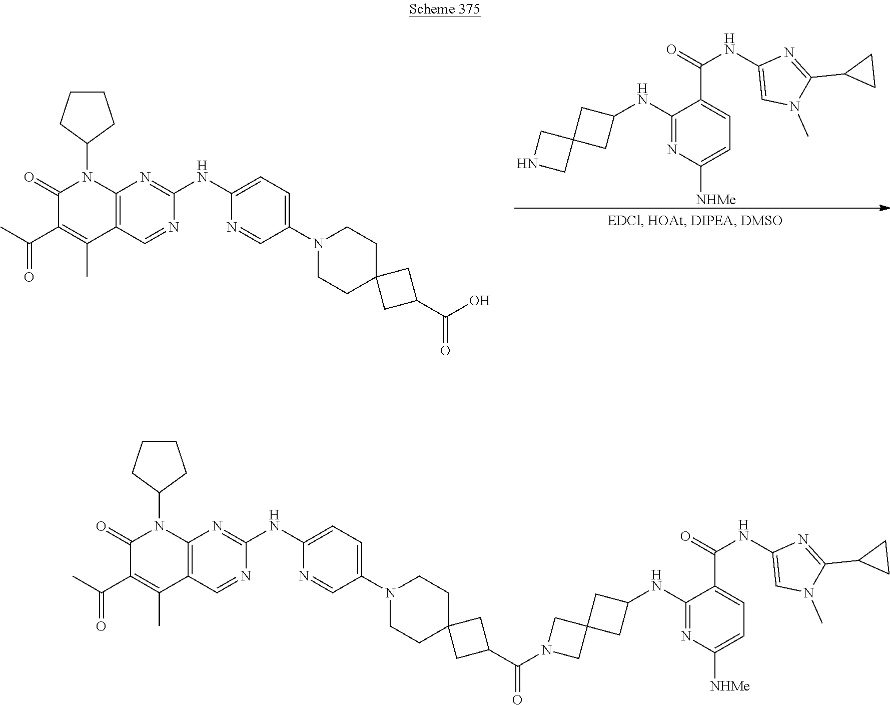 [SMILES: CC(=O)c1c(C)c2cnc(Nc3ccc(N4CCC5(CC4)CC(C(=O)O)C5)cn3)nc2n(C2CCCC2)c1=O ; CNc1ccc(C(=O)Nc2cn(C)c(C3CC3)n2)c(NC2CC3(CNC3)C2)n1 ; CNc1ccc(C(=O)Nc2cn(C)c(C3CC3)n2)c(NC2CC3(C2)CN(C(=O)C2CC4(CCN(c5ccc(Nc6ncc7c(C)c(C(C)=O)c(=O)n(C8CCCC8)c7n6)nc5)CC4)C2)C3)n1]

CPD-321 was synthesized following the standard procedure for preparing CPD-143 (3.5 mg, 35% yield) as a yellow solid. MS (ESI) m/z=894.7 [M+H]⁺.

### Example 376. 2-((2-(1-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)cyclopropane-1-carbonyl)-2-azaspiro[3.3]heptan-6-yl)amino)-N-(2-cyclopropyl-1-methyl-1H-imidazol-4-yl)-6-(isopropylamino)nicotinamide (CPD-322)

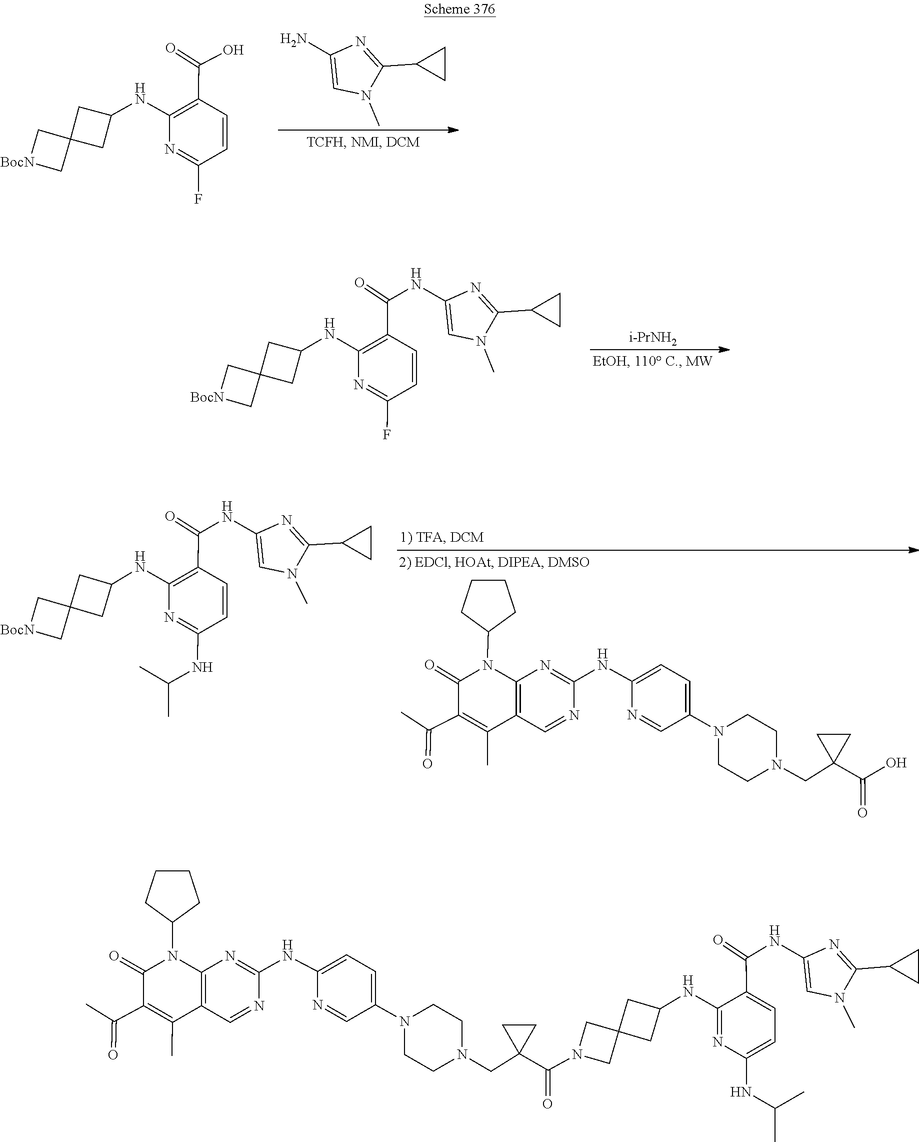

CPD-322 was synthesized following the standard procedures for preparing CPD-144 (0.53 mg, 2% yield over 4 steps) as a yellow solid. MS (ESI) m/z=937.7 [M+H]⁺.

### Example 377. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(2-cyclopropyl-1-methyl-1H-imidazol-4-yl)-6-(isopropylamino)nicotinamide (CPD-323)

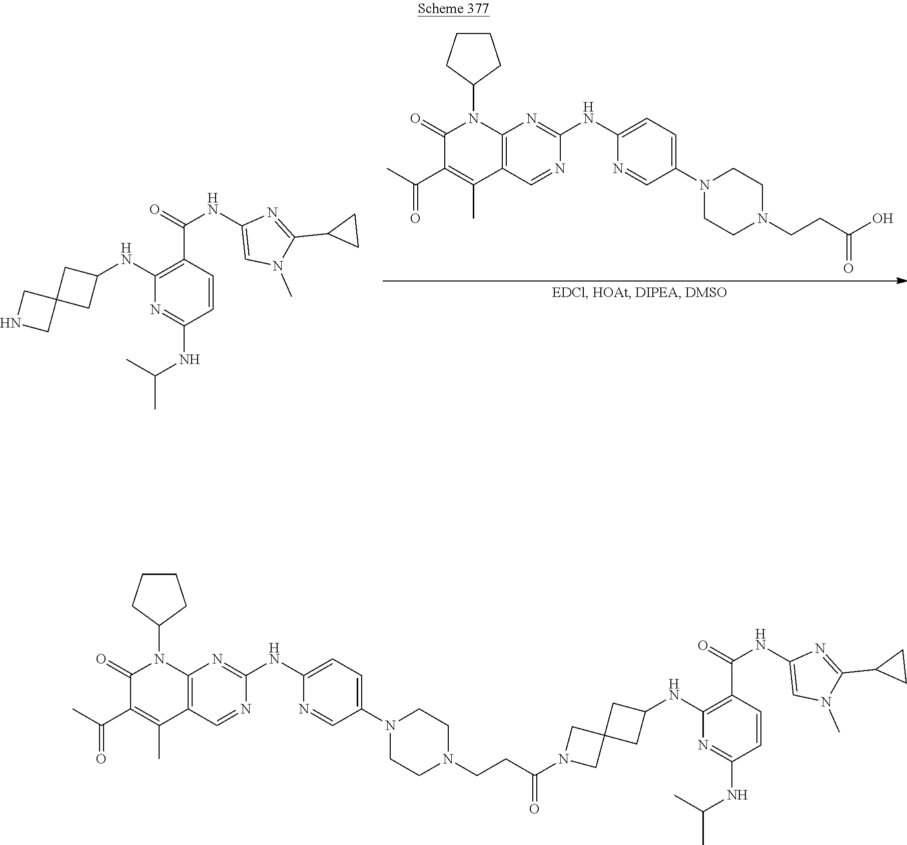 [SMILES: CC(C)Nc1ccc(C(=O)Nc2cn(C)c(C3CC3)n2)c(NC2CC3(CNC3)C2)n1 ; CC(=O)c1c(C)c2cnc(Nc3ccc(N4CCN(CCC(=O)O)CC4)cn3)nc2n(C2CCCC2)c1=O ; CC(=O)c1c(C)c2cnc(Nc3ccc(N4CCN(CCC(=O)N5CC6(CC(Nc7nc(NC(C)C)ccc7C(=O)Nc7cn(C)c(C8CC8)n7)C6)C5)CC4)cn3)nc2n(C2CCCC2)c1=O]

CPD-323 was synthesized following the standard procedure for preparing CPD-143 (1.8 mg, 20% yield) as a yellow solid. MS (ESI) m/z=911.6 [M+H]⁺.

### Example 378. 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(6-(dimethylamino)pyridazin-3-yl)-6-(methylamino)nicotinamide (CPD-324)

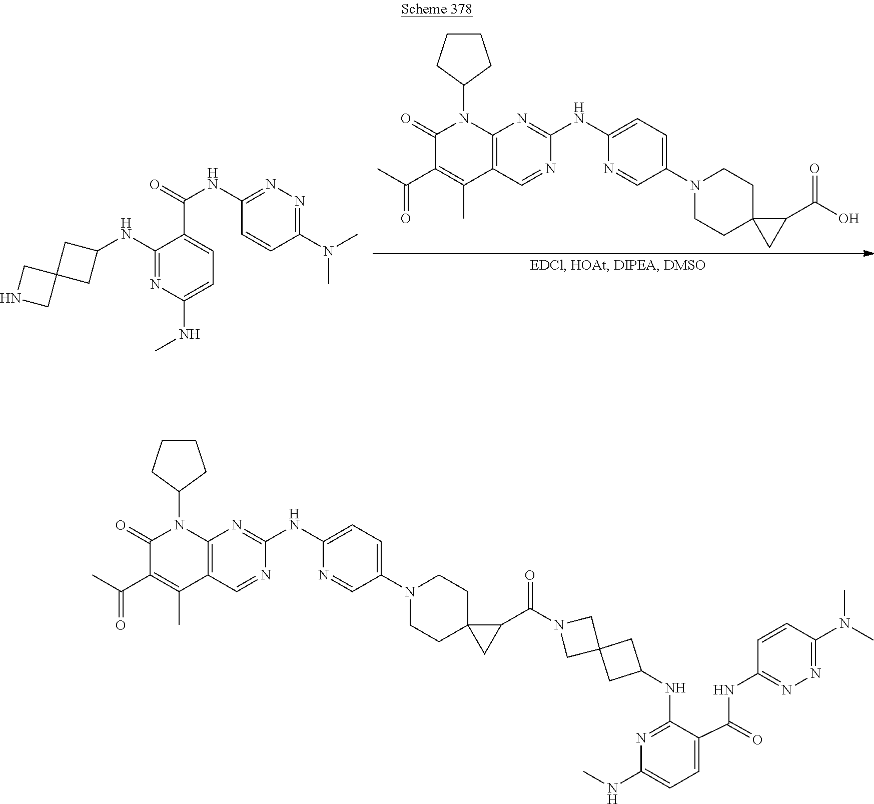 [SMILES: CNc1ccc(C(=O)Nc2ccc(N(C)C)nn2)c(NC2CC3(CNC3)C2)n1 ; CC(=O)c1c(C)c2cnc(Nc3ccc(N4CCC5(CC4)CC5C(=O)O)cn3)nc2n(C2CCCC2)c1=O ; CNc1ccc(C(=O)Nc2ccc(N(C)C)nn2)c(NC2CC3(C2)CN(C(=O)C2CC24CCN(c2ccc(Nc5ncc6c(C)c(C(C)=O)c(=O)n(C7CCCC7)c6n5)nc2)CC4)C3)n1]

CPD-324 was synthesized following the standard procedure for preparing CPD-143 (8.1 mg, 39% yield) as a yellow solid. MS (ESI) m/z=881.5 [M+H]⁺.

### Example 379. 2-((2-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)-2-azaspiro[3.3]heptan-6-yl)amino)-N-(6-(dimethylamino)pyridazin-3-yl)-6-(methylamino)nicotinamide (CPD-325)

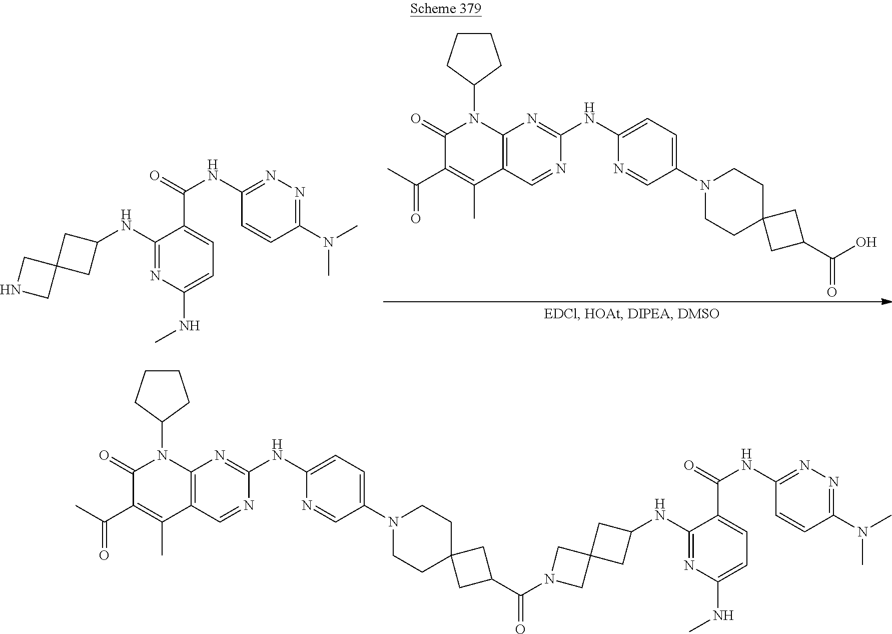

CPD-325 was synthesized following the standard procedure for preparing CPD-143 (6.2 mg, 38% yield)

### Example 380. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(6-(dimethylamino)pyridazin-3-yl)-6-(isopropylamino)nicotinamide (CPD-326)

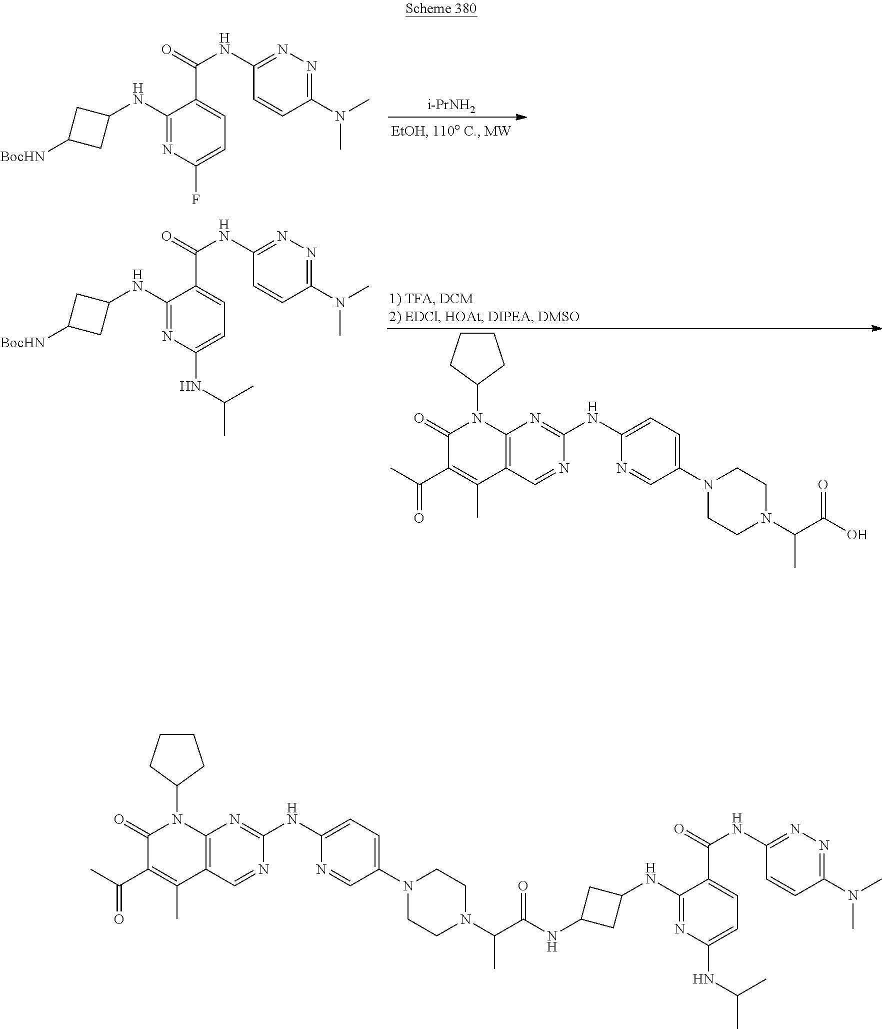

CPD-326 was synthesized following the standard procedures for preparing CPD-144 (1.5 mg, 5% yield over 3 steps) as a yellow solid. MS (ESI) m/z=886.5 [M+H]⁺.

### Example 381. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)cyclobutyl)amino)-N-(2-cyclopropyl-1-methyl-1H-imidazol-4-yl)-6-(isopropylamino)nicotinamide (CPD-327)

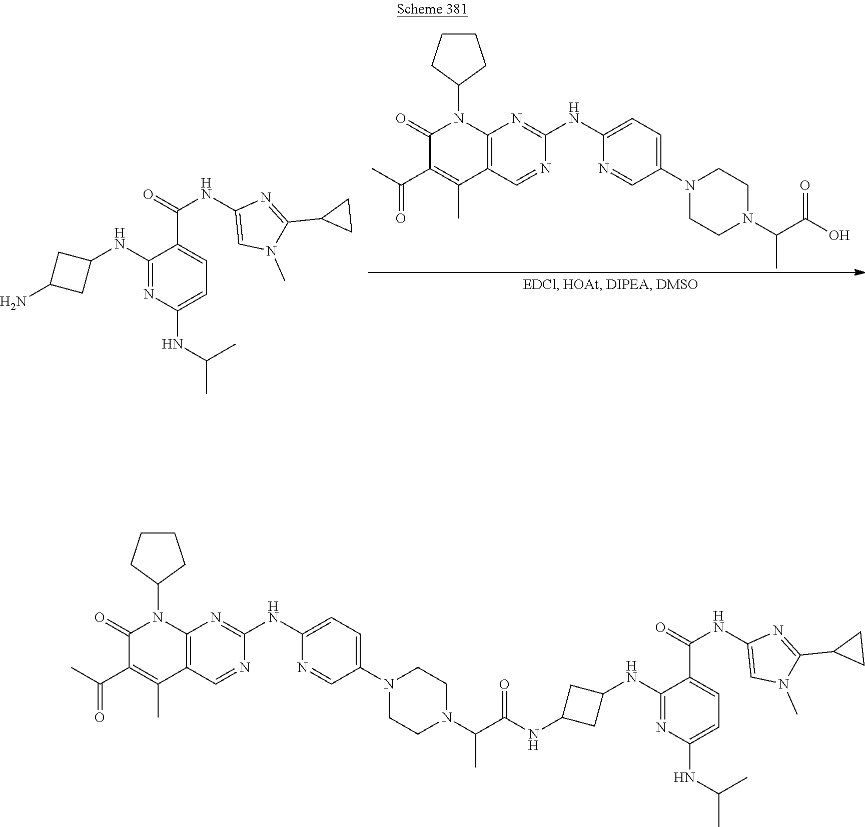

CPD-327 was synthesized following the standard procedure for preparing CPD-143 (5.7 mg, 24% yield) as a yellow solid. MS (ESI) m/z=885.7 [M+H]⁺.

### Example 382. 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(6-(dimethylamino)pyridazin-3-yl)-6-(methylamino)nicotinamide (CPD-328)

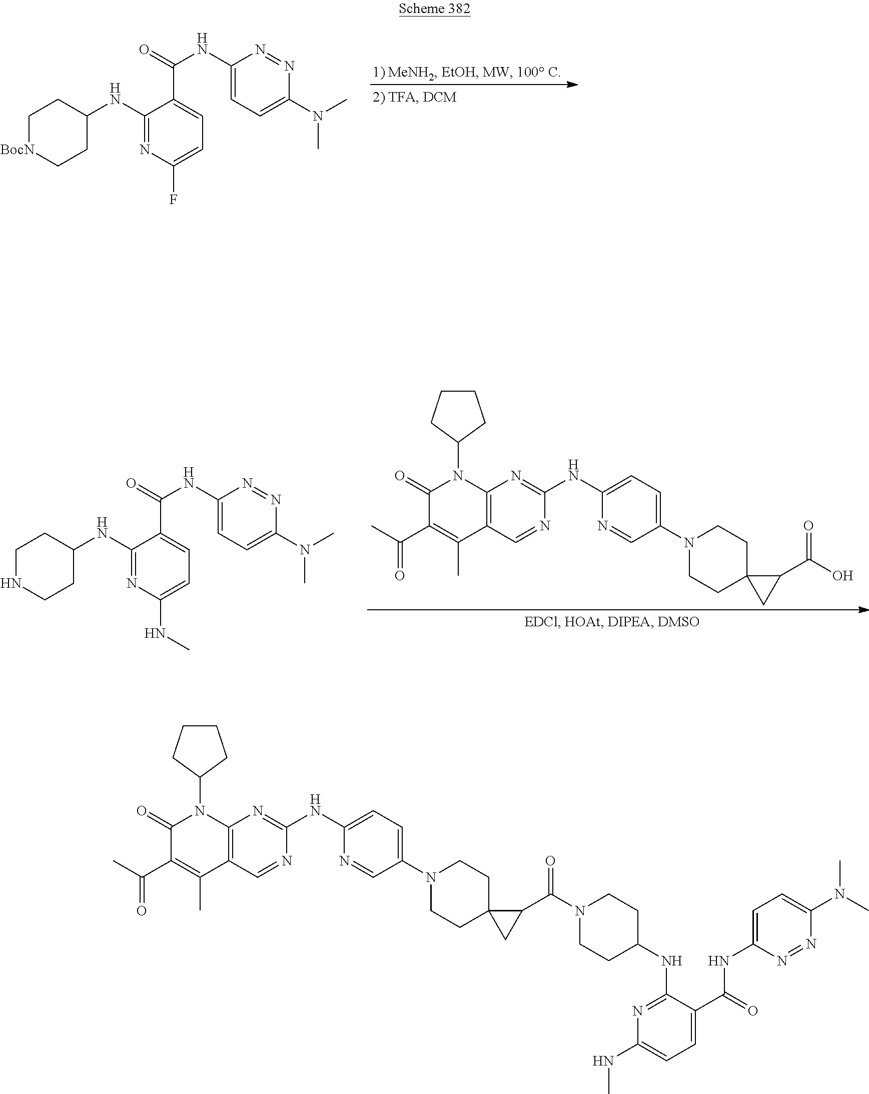

CPD-328 was synthesized following the standard procedures for preparing CPD-144 (8.0 mg, 13% yield over 3 steps) as a yellow solid. MS (ESI) m/z=869.6 [M+H]⁺.

### Example 383. 2-((1-(7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonane-2-carbonyl)piperidin-4-yl)amino)-N-(6-(dimethylamino)pyridazin-3-yl)-6-(methylamino)nicotinamide (CPD-329)

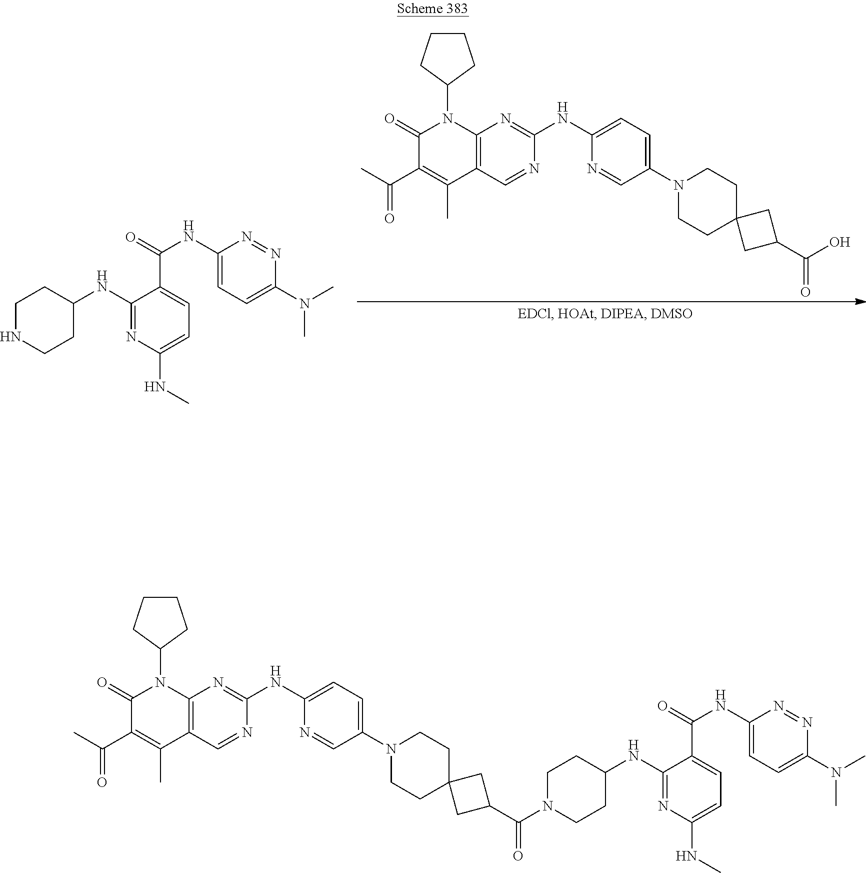 [SMILES: CNc1ccc(C(=O)Nc2ccc(N(C)C)nn2)c(NC2CCNCC2)n1 ; CC(=O)c1c(C)c2cnc(Nc3ccc(N4CCC5(CC4)CC(C(=O)O)C5)cn3)nc2n(C2CCCC2)c1=O ; CNc1ccc(C(=O)Nc2ccc(N(C)C)nn2)c(NC2CCN(C(=O)C3CC4(CCN(c5ccc(Nc6ncc7c(C)c(C(C)=O)c(=O)n(C8CCCC8)c7n6)nc5)CC4)C3)CC2)n1]

CPD-329 was synthesized following the standard procedure for preparing CPD-143 (2.0 mg, 8% yield) as a yellow solid. MS (ESI) m/z=883.7 [M+±H]t.

### Example 384. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-(1-hydroxyethyl)-1-methyl-1H-imidazol-4-yl)-6-(methylamino)nicotinamide (CPD-330)

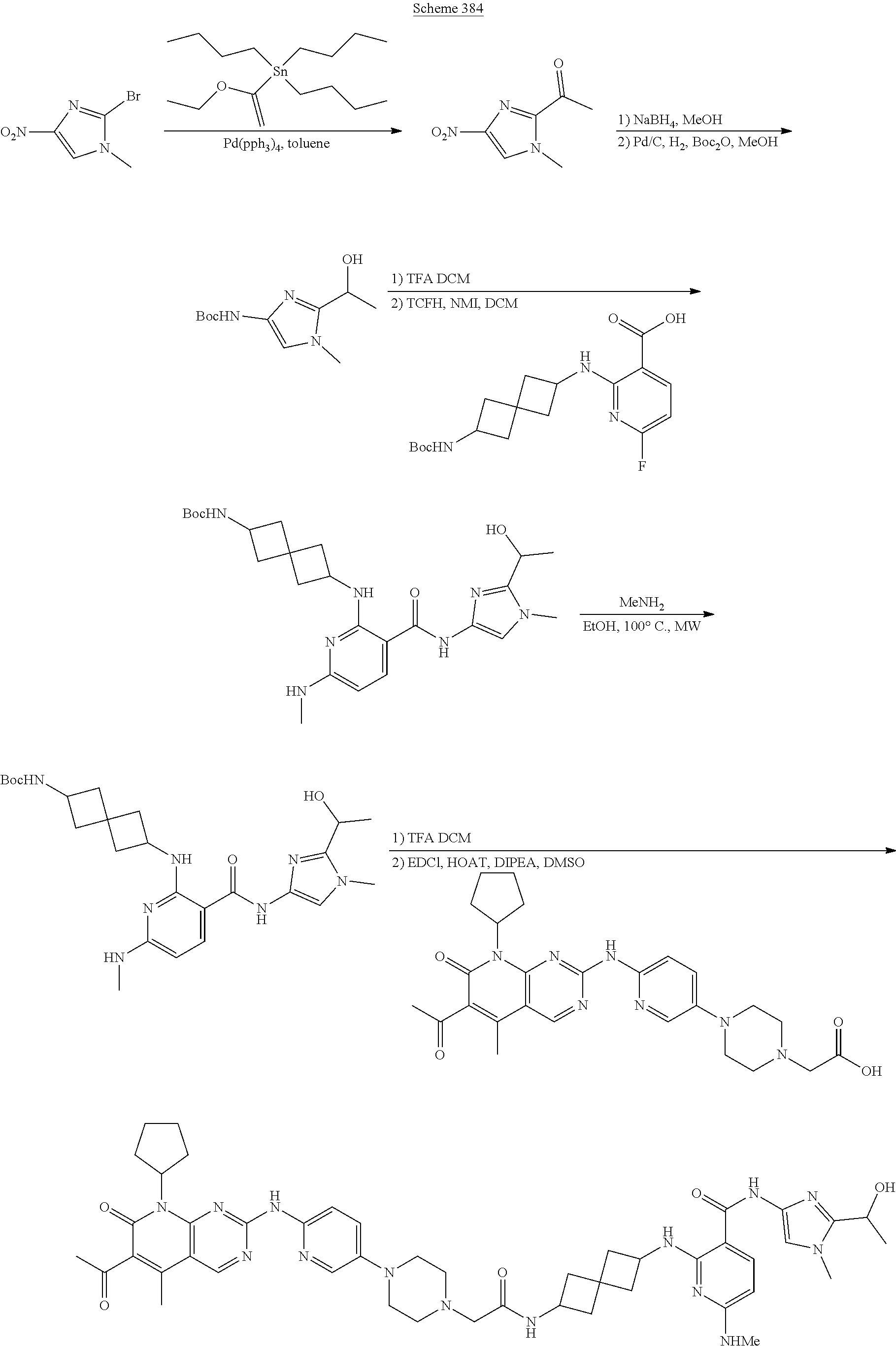

### Step 1. Synthesis of 1-(1-methyl-4-nitro-1H-imidazol-2-yl)ethan-1-one

To a solution of 2-bromo-1-methyl-4-nitro-1H-imidazole (470 mg, 2.28 mmol) and tributyl(1-ethoxyvinyl)stannane (1.07 g, 2.97 mmol) in toluene (10 mL) was added Pd(PPh₃)₄ (132 mg, 0.11 mmol). The reaction mixture was stirred at 100° C. for 16 h. The resulting solution was partitioned between a saturated aqueous ammonium chloride solution and ethyl acetate. The organic layer was concentrated, the residue was re-dissolved in THF (10 mL) and aq. HCl (2 N, 10 mL). The resulting solution was stirred at rt for 2 h. The mixture was extracted with ethyl acetate (3×10 mL). The combined organic layers were washed with brine (10 mL), dried over anhydrous Na₂SO₄, filtered and concentrated. The residue was purified by flash chromatography to give the desired product (260 mg, 67% yield) as a white solid.

### Step 2. Synthesis of 1-(1-methyl-4-nitro-1H-imidazol-2-yl)ethan-1-ol

To a solution of 1-(1-methyl-4-nitro-1H-imidazol-2-yl)ethan-1-one (50 mg, 0.29 mmol) in MeOH (2 mL) was added NaBH₄ (22 mg, 0.59 mmol). The reaction mixture was stirred at rt for 1 h. The reaction mixture was concentrated to give the crude product.

### Step 3. Synthesis of tert-butyl (2-(1-hydroxyethyl)-1-methyl-1H-imidazol-4-yl)carbamate

To a solution of 1-(1-methyl-4-nitro-1H-imidazol-2-yl)ethan-1-ol (50 mg, 0.29 mmol) in MeOH (5 mL) were added Boc₂O (645 mg, 2.96 mmol) and 10% Pd/C (14 mg). The reaction mixture was stirred at rt for 3 h under hydrogen balloon. The reaction mixture was filtered and concentrated. The resulting residue was purified by silica gel flash chromatography to give the desired product (43 mg, 60% yield) as a white solid. MS (ESI) m/z=242.5 [M+H]⁺.

### Step 4. Synthesis of 1-(4-amino-1-methyl-1H-imidazol-2-yl)ethan-1-ol

To a solution of tert-butyl (2-(1-hydroxyethyl)-1-methyl-1H-imidazol-4-yl)carbamate (45 mg, 0.18 mmol) in dichloromethane (1 mL) was added TFA (1 mL). The reaction mixture was stirred at rt for 1 h. The reaction mixture was concentrated to give the desired product (45 mg, 94% yield) as a yellow oil. This compound was used directly in the next step without further purification. MS (ESI) m/z=142.2 [M+H]⁺.

### Step 5. Synthesis of tert-butyl (6-((6-fluoro-3-((2-(1-hydroxyethyl)-1-methyl-1H-imidazol-4-yl)carbamoyl)pyridin-2-yl)amino)spiro[3.3]heptan-2-yl)carbamate

A mixture of 2-((6-((tert-butoxycarbonyl)amino)spiro[3.3]heptan-2-yl)amino)-6-fluoronicotinic acid (30 mg, 0.08 mmol), 1-(4-amino-1-methyl-1H-imidazol-2-yl)ethan-1-ol (42 mg, 0.14 mmol), TCFH (46 mg, 0.16 mmol) and NMI (20 mg, 0.24 mmol) in dichloromethane (5 mL) was stirred at rt for 1 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by silica gel flash chromatography to give the desired product (32 mg, 79% yield) as a colorless oil. MS (ESI) m/z=489.5 [M+H]⁺.

### Step 6. Synthesis of tert-butyl (6-((3-((2-(1-hydroxyethyl)-1-methyl-1H-imidazol-4-yl)carbamoyl)-6-(methylamino)pyridin-2-yl)amino)spiro[3.3]heptan-2-yl)carbamate

To a solution of tert-butyl (6-((6-fluoro-3-((2-(1-hydroxyethyl)-1-methyl-1H-imidazol-4-yl)carbamoyl)pyridin-2-yl)amino)spiro[3.3]heptan-2-yl)carbamate (32 mg, 0.06 mmol) in EtOH (1 mL) was added methanamine (30% in EtOH, 1 mL). The reaction mixture was stirred at 100° C. for 1 h under microwave irradiation. The reaction mixture was concentrated and purified by silica gel flash chromatography to give the desired product (25 mg, 76% yield) as a white solid. MS (ESI) m/z=500.5 [M+H]⁺.

### Step 7. Synthesis of 2-((6-aminospiro[3.3]heptan-2-yl)amino)-N-(2-(1-hydroxyethyl)-1-methyl-1H-imidazol-4-yl)-6-(methylamino)nicotinamide

To a solution of tert-butyl (6-((3-((2-(1-hydroxyethyl)-1-methyl-1H-imidazol-4-yl)carbamoyl)-6-(methylamino)pyridin-2-yl)amino)spiro[3.3]heptan-2-yl)carbamate (25 mg, 0.05 mmol) in dichloromethane (1 mL) was added TFA (1 mL). The reaction mixture was stirred at rt for 1 h. The mixture was concentrated to give the desired product (25 mg, 97% yield) as a yellow oil. This compound was used directly in the next step without further purification. MS (ESI) m/z=400.4 [M+H]⁺.

### Step 8. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-(1-hydroxyethyl)-1-methyl-1H-imidazol-4-yl)-6-(methylamino)nicotinamide

A mixture of 2-((6-aminospiro[3.3]heptan-2-yl)amino)-N-(2-(1-hydroxyethyl)-1-methyl-1H-imidazol-4-yl)-6-(methylamino)nicotinamide (25 mg, 0.05 mmol), 5-((3-((6-(dimethylamino)pyridazin-3-yl)carbamoyl)-6-(methylamino)pyridin-2-yl)amino)pentanoic acid (25 mg, 0.05 mmol), EDCI (14 mg, 0.07 mmol), HOAT (10 mg, 0.07 mmol) and DIPEA (31 mg, 0.24 mmol) in DMSO (2 mL) was stirred at rt for 16 h. The reaction mixture was purified by reverse-phase chromatography and pre-HPLC to give the desired product (6.0 mg, 12% yield) as a yellow solid. MS (ESI) m/z=887.7 [M+H]⁺.

### Example 385. 2-((3-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonyl)cyclobutyl)amino)-N-(6-(dimethylamino)pyridazin-3-yl)-6-(methylamino)nicotinamide (CPD-331)

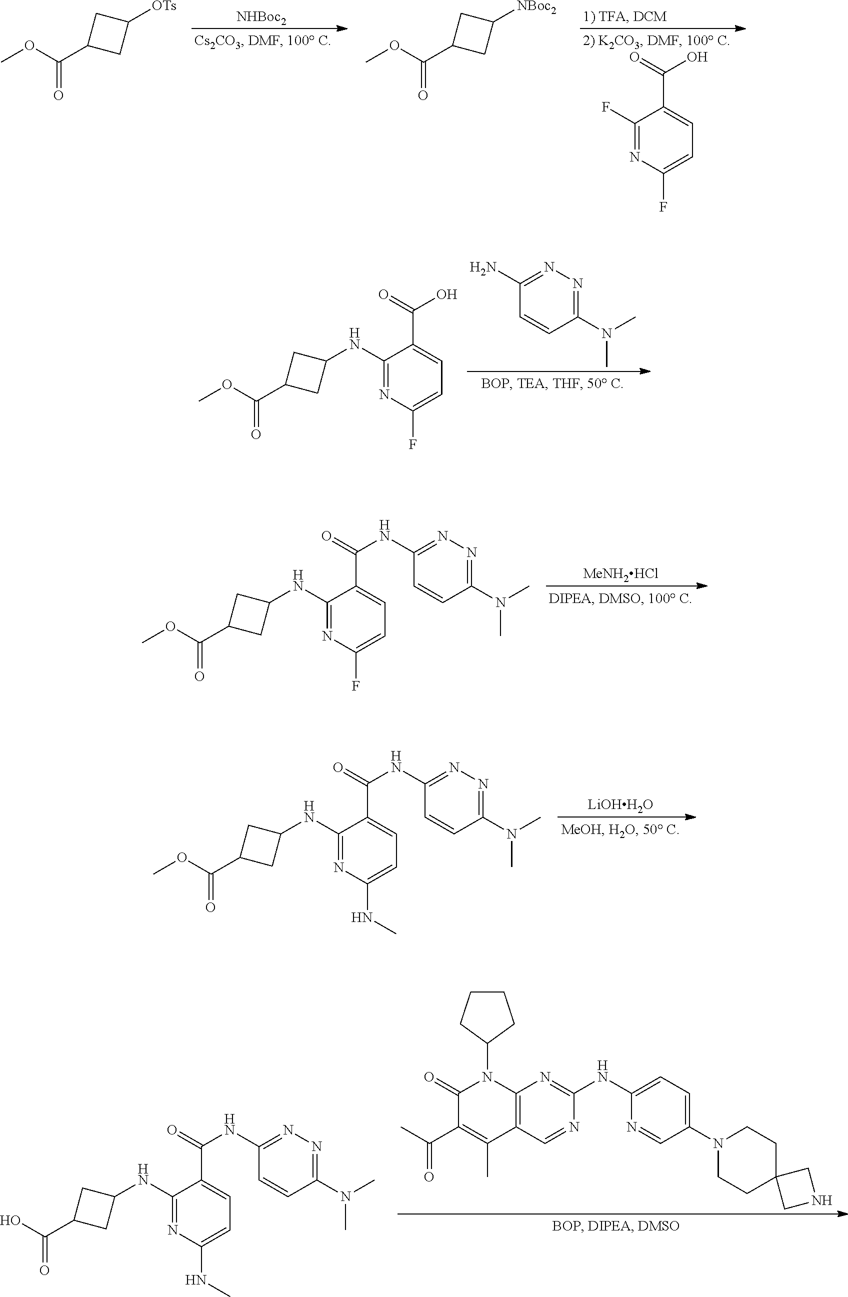

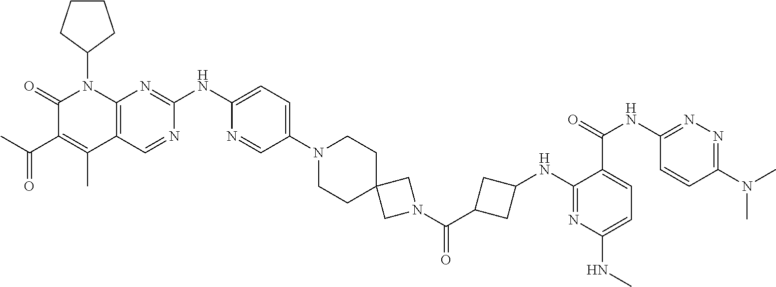 [SMILES: CNc1ccc(C(=O)Nc2ccc(N(C)C)nn2)c(NC2CC(C(=O)N3CC4(CCN(c5ccc(Nc6ncc7c(C)c(C(C)=O)c(=O)n(C8CCCC8)c7n6)nc5)CC4)C3)C2)n1]

### Step 1. Synthesis of methyl 3-[bis(tert-butoxycarbonyl)amino]cyclobutanecarboxylate

To a solution of methyl 3-(tosyloxy)cyclobutane-1-carboxylate (1.5 g, 5.28 mmol) in DMF (10 mL) were added tert-butyl N-tert-butoxycarbonylcarbamate (1.49 g, 6.86 mmol) and Cs₂CO₃ (3.44 g, 10.55 mmol).

The reaction mixture was stirred at 100° C. for 8 h, before it was poured into water (100 mL) and extracted with ethyl acetate (2×100 mL). The combined organic extracts were washed with saturated brine (80 mL), dried over anhydrous sodium sulfate, filtered, and evaporated under reduced pressure. The residue was purified by silica gel chromatography to get the product (1.2 g, 69% yield) as a light-yellow oil. MS (ESI) m/z=130.1 [M+H-200]⁺.

The remaining steps were performed according to the standard procedures for preparing CPD-82 to give the desired product (8 mg, 14% yield over 6 steps) as a yellow solid. MS (ESI) m/z=855.7 [M+H]⁺.

### Example 386. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)cyclobutyl)amino)-N-(6-(dimethylamino)pyridazin-3-yl)-6-(methylamino)nicotinamide (CPD-332)

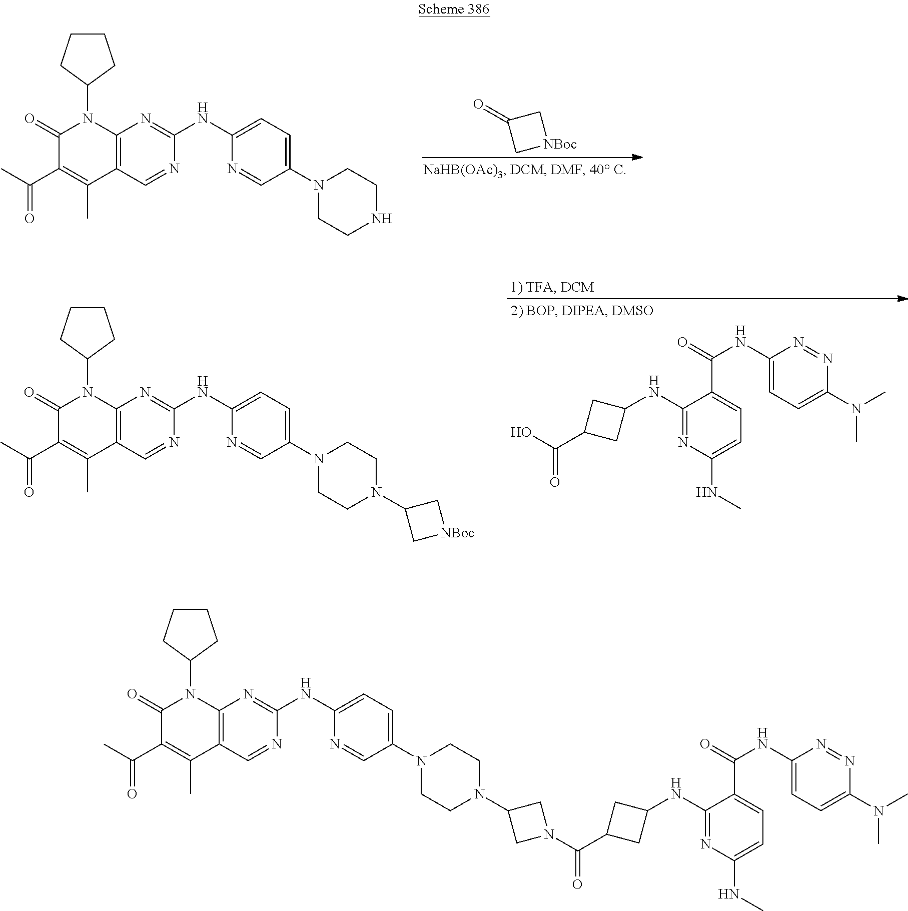

### Step 1. Synthesis of tert-butyl 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carboxylate

To a solution of 6-acetyl-8-cyclopentyl-5-methyl-2-((5-(piperazin-1-yl)pyridin-2-yl)amino)pyrido[2,3-d]pyrimidin-7(8H)-one (100 mg, 0.22 mmol) in dichloromethane (10 mL) and DMF (2 mL) were added tert-butyl 3-oxoazetidine-1-carboxylate (76 mg, 0.44 mmol) and NaHB(OAc)₃ (150 mg, 0.67 mmol). The mixture was stirred at 40° C. for 4 h, before it was poured into water (50 mL) and extracted with ethyl acetate (2×50 mL). The combined organic extracts were washed with saturated brine (30 mL), dried over anhydrous sodium sulfate, filtered, and evaporated under reduced pressure. The residue was purified by silica gel chromatography to get product (100 mg, 74% yield) as a yellow solid. MS (ESI) m/z=603.6 [M+H]⁺.

### Step 2. Synthesis of 6-acetyl-2-((5-(4-(azetidin-3-yl)piperazin-1-yl)pyridin-2-yl)amino)-8-cyclopentyl-5-methylpyrido[2,3-d]pyrimidin-7(8H)-one

To a solution of tert-butyl 3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carboxylate (100 mg, 0.17 mmol) in dichloromethane (3 mL) was added TFA (1 mL). The mixture was stirred at rt for 4 h, before it was evaporated under reduced pressure to get the product (80 mg, 96% yield) as a yellow solid, which was used directly in the next step without further purification. MS (ESI) m/z=503.5 [M+H]⁺.

### Step 3. Synthesis of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)cyclobutyl)amino)-N-(6-(dimethylamino)pyridazin-3-yl)-6-(methylamino)nicotinamide

To a solution of 6-acetyl-2-((5-(4-(azetidin-3-yl)piperazin-1-yl)pyridin-2-yl)amino)-8-cyclopentyl-5-methylpyrido[2,3-d]pyrimidin-7(8H)-one (25 mg, 49.74 μmol) in DMSO (2 mL) were added 3-[[3-[[6-(dimethylamino)pyridazin-3-yl]carbamoyl]-6-(methylamino)-2-pyridyl]amino]cyclobutanecarboxylic acid (23 mg, 59.69 mol), DIPEA (32 mg, 0.25 mmol) and BOP (44 mg, 99.48 μmol). The mixture was stirred at 40° C. for 2 h, before it was purified by prep-HPLC to afford the title compound (10 mg, 23% yield) as a yellow solid. MS (ESI) m/z=870.6 [M+H]⁺.

### Example 387. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-(methylamino)nicotinamide (CPD-333)

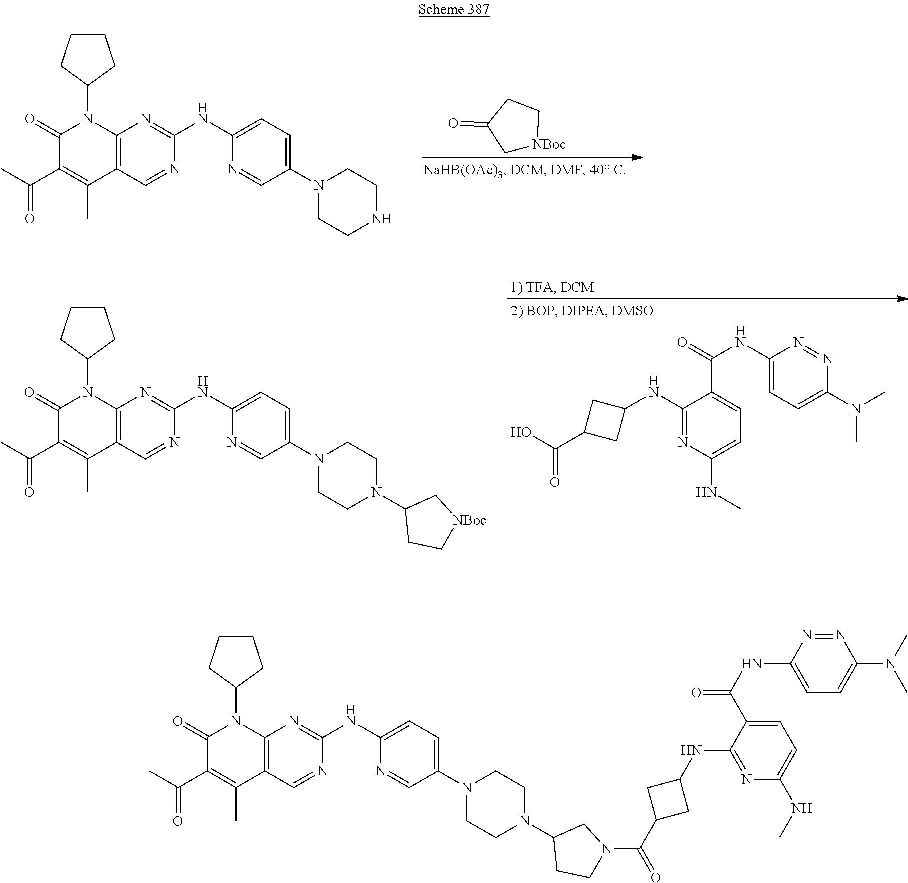

CPD-333 was synthesized following the standard procedures for preparing CPD-332 (10.0 mg, 16% yield over 3 steps) as a yellow solid. MS (ESI) m/z=884.6 [M+H]⁺.

### Example 388. 2-((3-(4-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)piperidine-1-carbonyl)cyclobutylamino)-N-(6-(dimethylamino)pyridazin-3-yl)-6-(methylamino)nicotinamide (CPD-334)

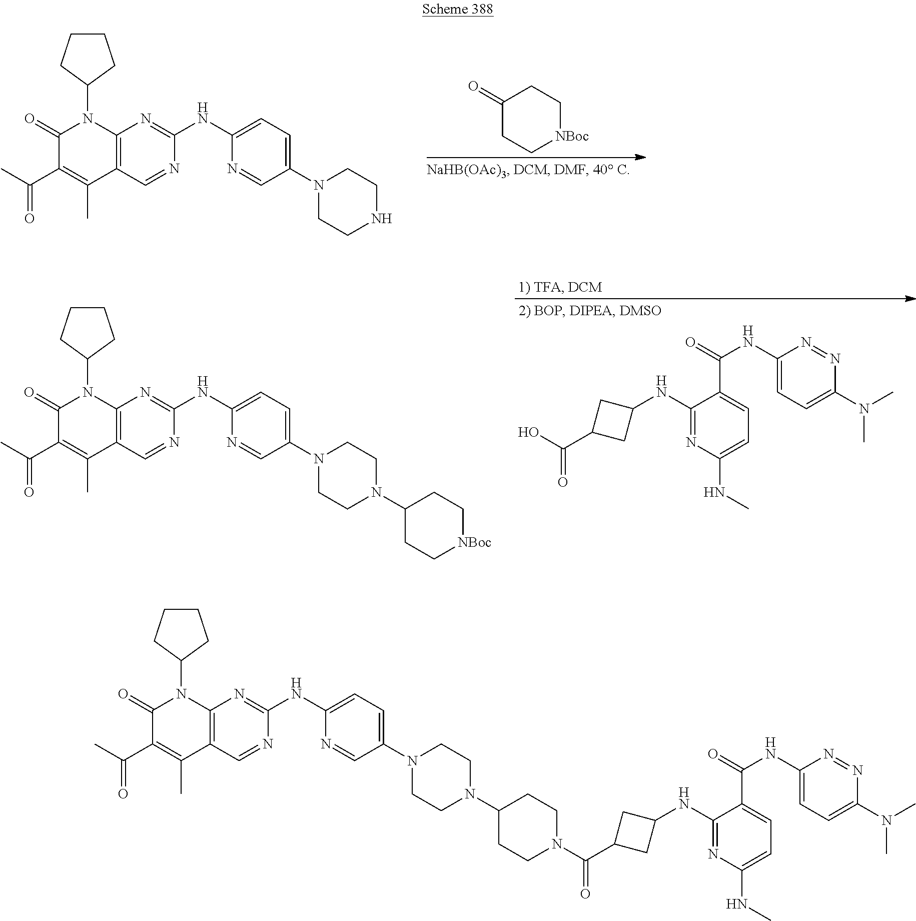

CPD-334 was synthesized following the standard procedures for preparing CPD-332 (8.0 mg, 14% yield over 3 steps) as a yellow solid. MS (ESI) m/z=898.6 [M+H]⁺.

### Example 389. 2-((3-(4-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)-1H-1,2,3-triazol-1-yl)cyclobutyl)amino)-N-(6-(dimethylamino)pyridazin-3-yl)-6-(methylamino)nicotinamide (CPD-335)

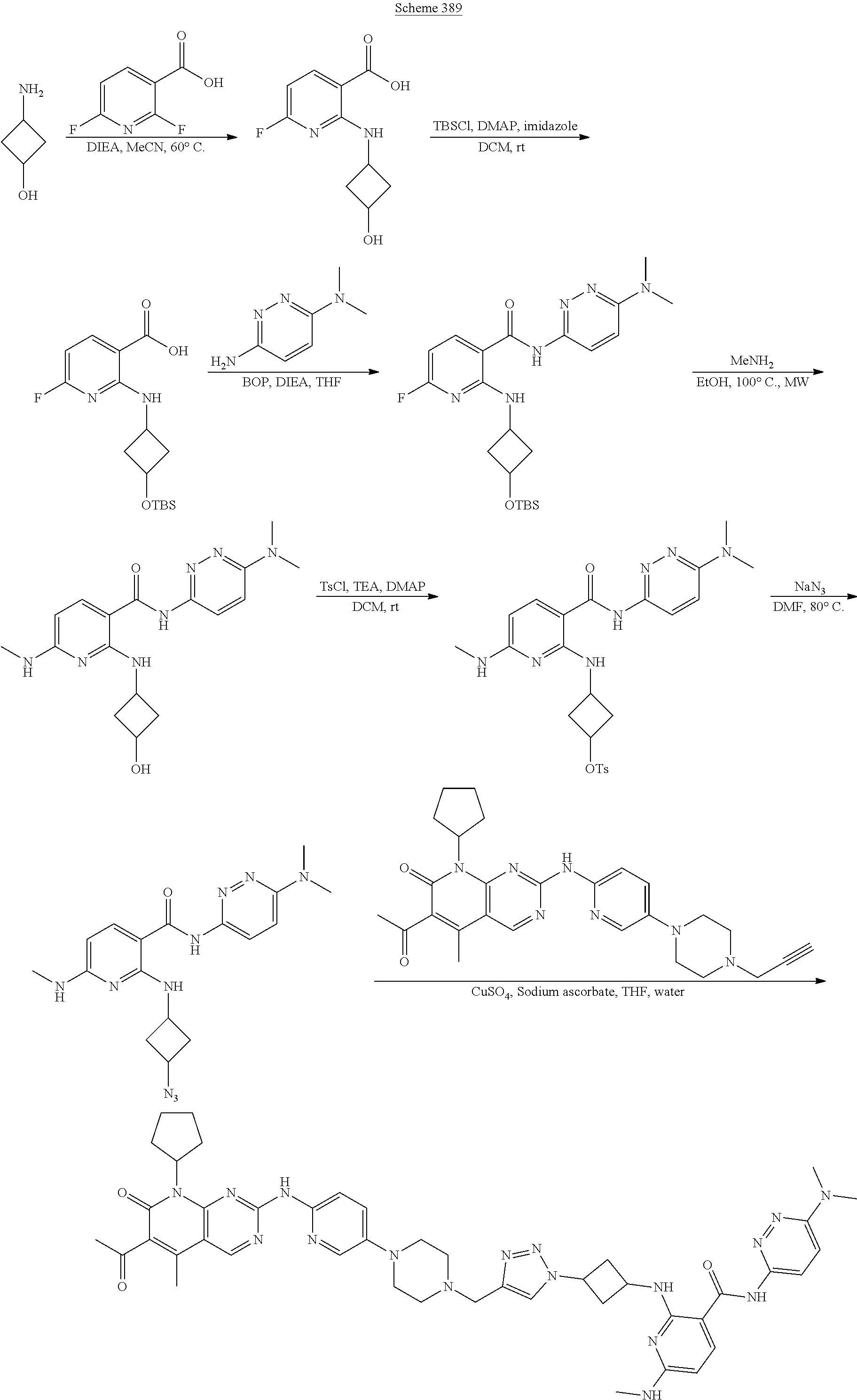

CPD-335 was synthesized following the standard procedures for preparing CPD-202 (1.5 mg, 1% yield over 7 steps) as a yellow solid. MS (ESI) m/z=868.5 [M+H]⁺.

### Example 390. 2-(((3-((3-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)methyl)pyrrolidin-1-yl)methyl)cyclobutyl)amino)-N-(6-(dimethylamino)pyridazin-3-yl)-6-(methylamino)nicotinamide (CPD-336)

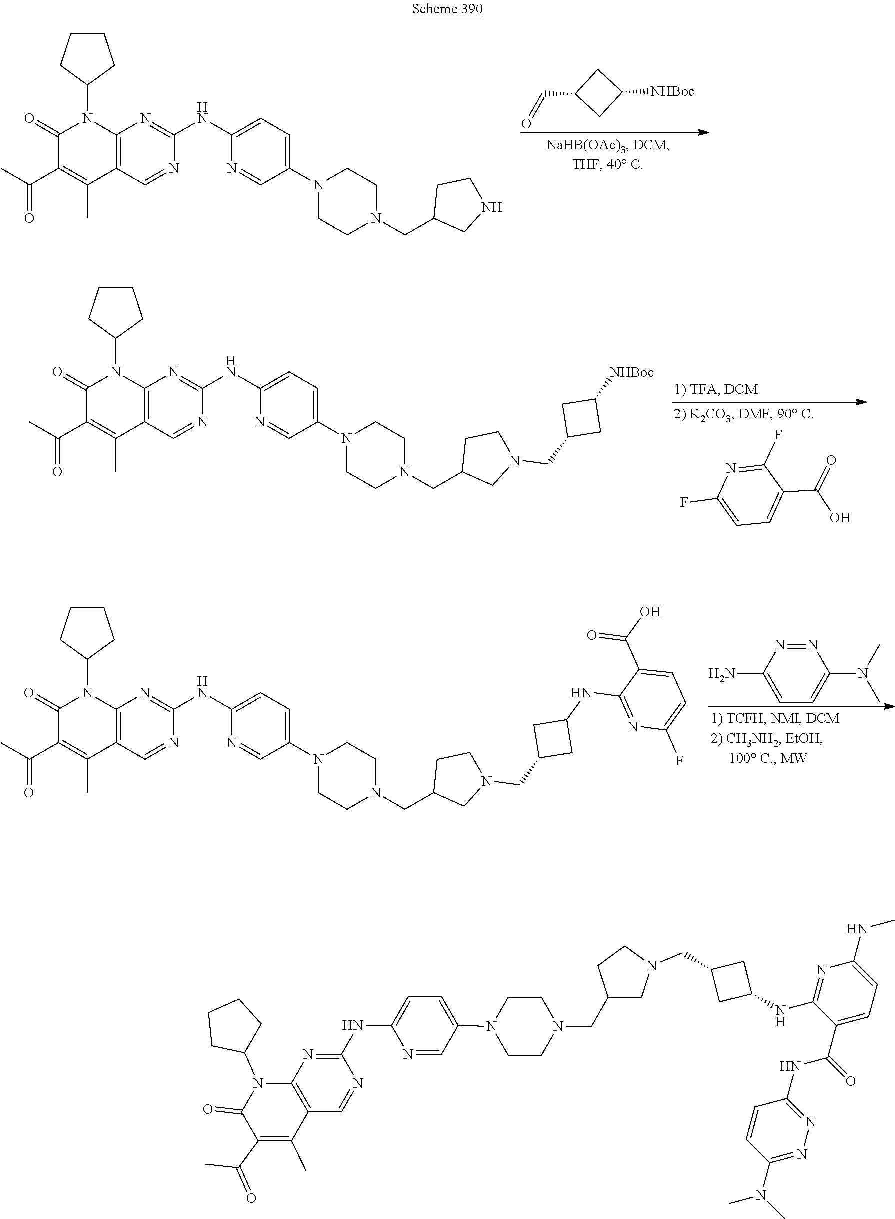

CPD-336 was synthesized following the standard procedures for preparing CPD-319 (2.0 mg, 100 yield over 5 steps) as a yellow solid. MS (ESI) m/z=884.5 [M+H]⁺.

### Example 391. 2-((3-(9-(6-((6-acetyl-8-cyclopentyl-5-methyl-7-oxo-7,8-dihydropyrido[2,3-d]pyrimidin-2-yl)amino)pyridin-3-yl)-3,9-diazaspiro[5.5]undecane-3-carbonyl)cyclobutyl)amino)-N-(6-(dimethylamino)pyridazin-3-yl)-6-(methylamino)nicotinamide (CPD-337)

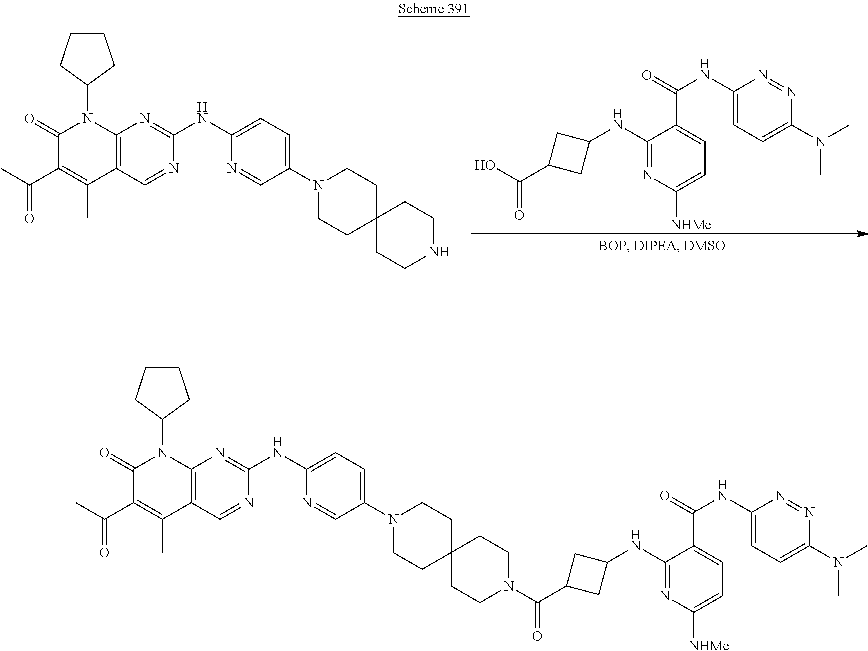 [SMILES: CC(=O)c1c(C)c2cnc(Nc3ccc(N4CCC5(CCNCC5)CC4)cn3)nc2n(C2CCCC2)c1=O ; CNc1ccc(C(=O)Nc2ccc(N(C)C)nn2)c(NC2CC(C(=O)O)C2)n1 ; CNc1ccc(C(=O)Nc2ccc(N(C)C)nn2)c(NC2CC(C(=O)N3CCC4(CC3)CCN(c3ccc(Nc5ncc6c(C)c(C(C)=O)c(=O)n(C7CCCC7)c6n5)nc3)CC4)C2)n1]

CPD-337 was synthesized following the standard procedure for preparing CPD-261 (5.0 mg, 34% yield) as a yellow solid. MS (ESI) m/z=883.6 [M+H]⁺.

### Example 392. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(2-cyclopropyl-1-methyl-1H-imidazol-4-yl)-6-(isopropylamino)nicotinamide (CPD-338)

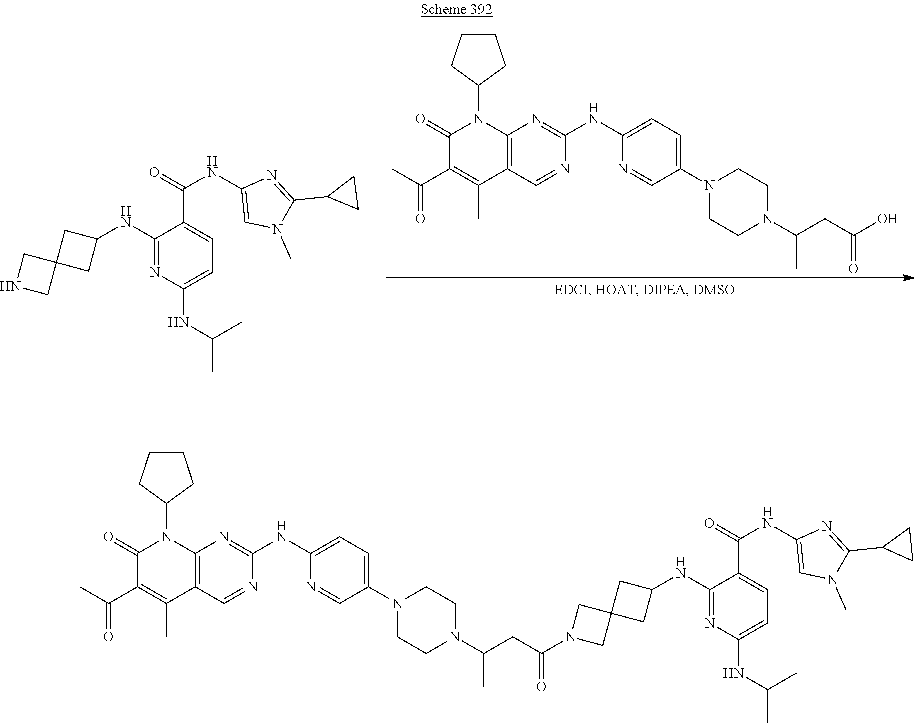

CPD-338 was synthesized following the standard procedure for preparing CPD-143 (3.0 mg, 18% yield) as a yellow solid. MS (ESI) m/z=925.8 [M+H]⁺.

### Example 393. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(2-cyclopropyl-1-methyl-1H-imidazol-4-yl)-6-((methyl-d₃)amino)nicotinamide (CPD-339)

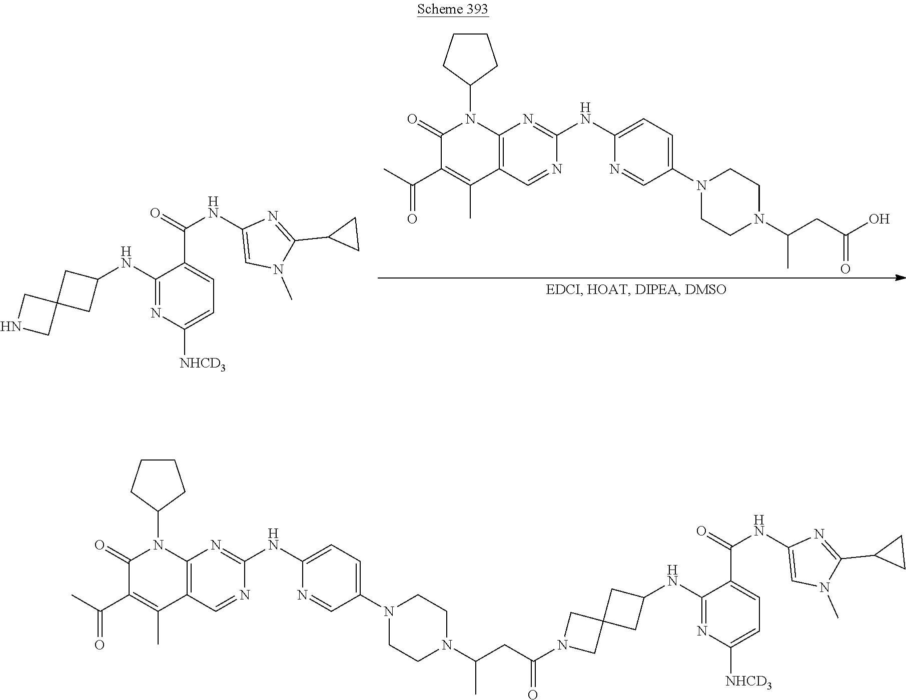

CPD-339 was synthesized following the standard procedure for preparing CPD-143 (2.0 mg, 25% yield) as a yellow solid. MS (ESI) m/z=900.7 [M+H]⁺.

### Example 394. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-(2-hydroxypropan-2-yl)-1-methyl-1H-imidazol-4-yl)-6-(methylamino)nicotinamide (CPD-340)

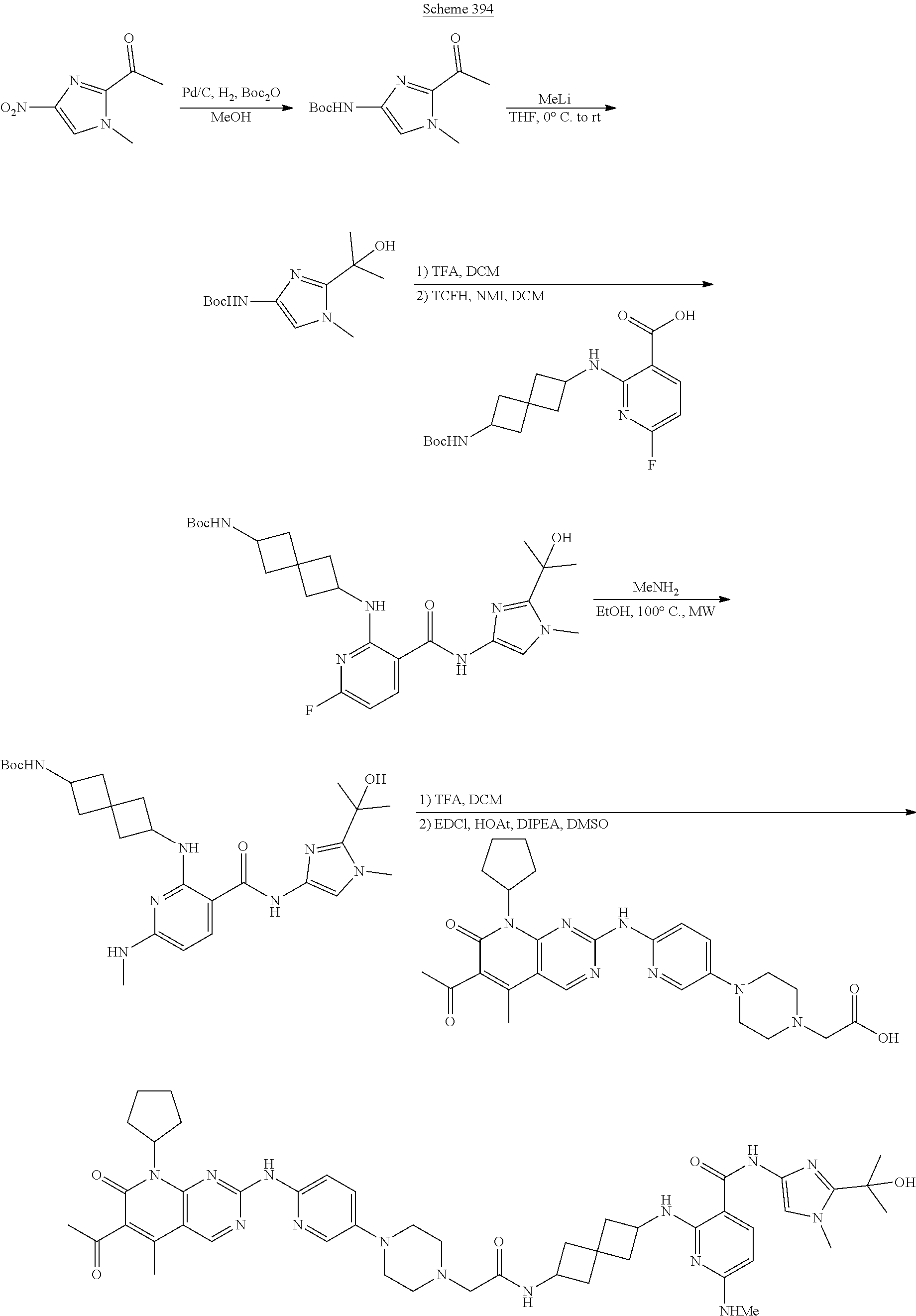

### Step 1. Synthesis of tert-butyl (2-acetyl-1-methyl-1H-imidazol-4-yl)carbamate

To a solution of 1-(1-methyl-4-nitro-1H-imidazol-2-yl)ethan-1-one (50 mg, 0.29 mmol) in MeOH (5 mL) were added Boc₂O (645 mg, 2.96 mmol) and 10% Pd/C (14 mg). The reaction mixture was stirred at rt for 16 h under hydrogen balloon. The reaction mixture was filtered and concentrated. The resulting residue was purified by silica gel flash chromatography to give the desired product (56 mg, 79% yield) as a white solid. MS (ESI) m/z=240.3 [M+H]⁺.

### Step 2. Synthesis of tert-butyl (2-(2-hydroxypropan-2-yl)-1-methyl-1H-imidazol-4-yl)carbamate

To a solution of tert-butyl (2-acetyl-1-methyl-1H-imidazol-4-yl)carbamate (56 mg, 0.23 mmol) in THF (5 mL) was added methyllithium (0.3 mL, 0.46 mmol) dropwise at 0° C. The reaction mixture was stirred at rt for 1 h. The reaction mixture was quenched with water (10 mL) and extracted with ethyl acetate (3×10 mL). The combined organic layers were washed with brine, dried over anhydrous Na₂SO₄, filtered and concentrated. The residue was purified by silica gel chromatography to give the desired product (31 mg, 51% yield) as a colorless oil. MS (ESI) m/z 256.3 [M+H]⁺.

The remaining steps were performed according to the procedures for preparing CPD-144 to provide the desired product (6.1 mg, 1% yield over 5 steps) as a yellow solid. MS (ESI) m/z=901.6 [M+H]⁺.

### Example 395. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-((methyl-d₃)amino)nicotinamide (CPD-341)

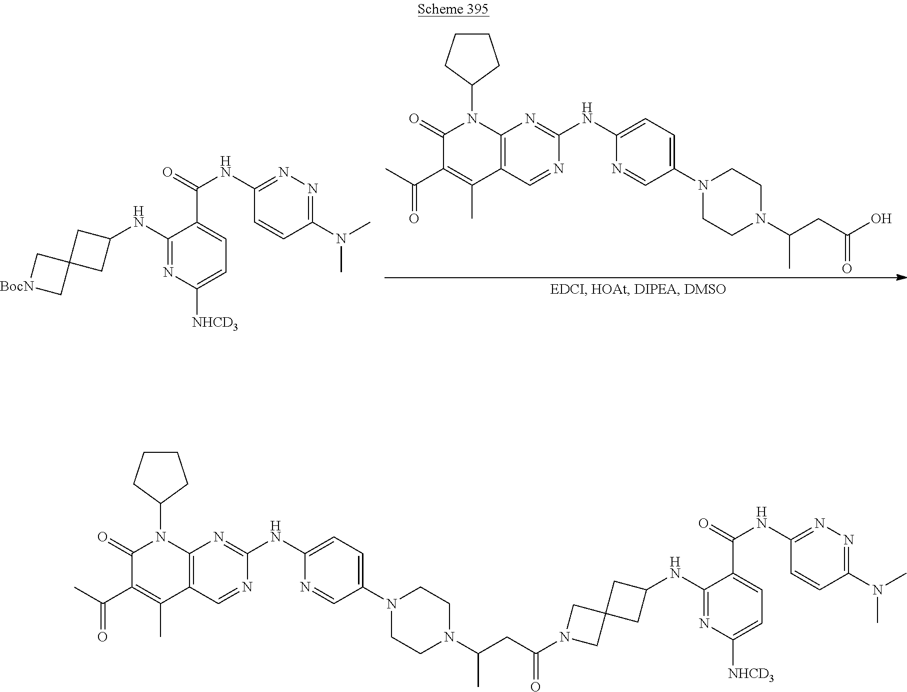

CPD-341 was synthesized following the standard procedure for preparing CPD-143 (3.4 mg, 19% yield) as a yellow solid. MS (ESI) m/z=901.7 [M+H]⁺.

### Example 396. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-(dimethylamino)pyridazin-3-yl)-6-((methyl-d₃)amino)nicotinamide (CPD-342)

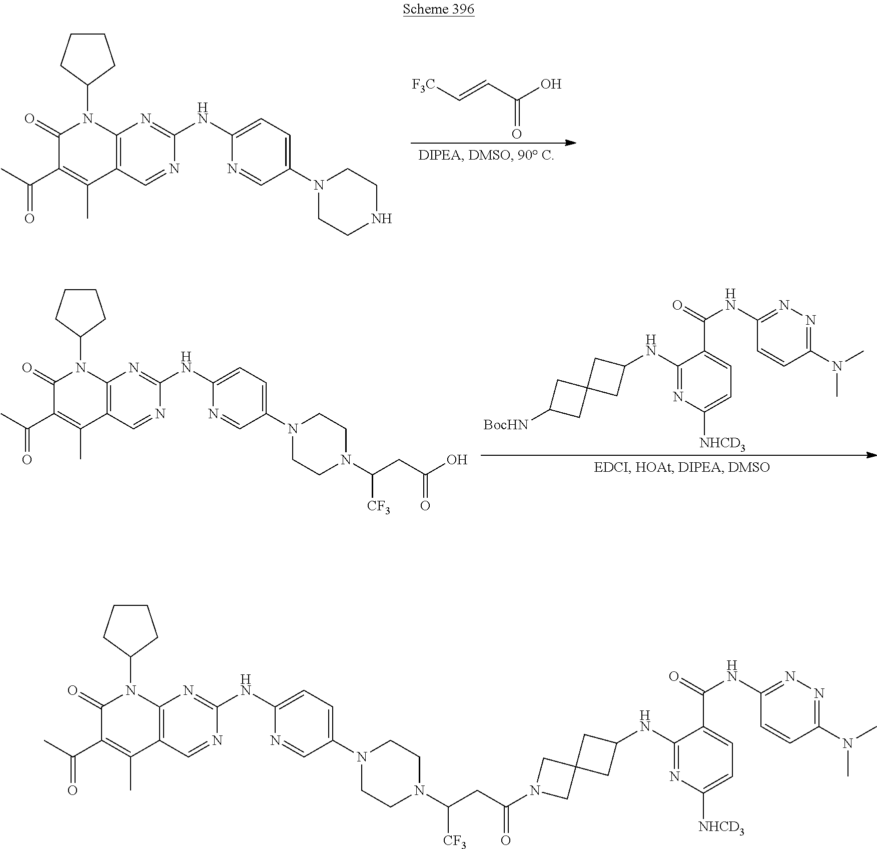

### Step 1. Synthesis of 3-(4-(6-((6-acetyl-8-cyclopentyl-5-methyl-7-oxo-7,8-dihydro pyrido[2,3-d]pyrimidin-2-yl)amino)pyridin-3-yl)piperazin-1-yl)-4,4,4-trifluorobutanoic acid

A mixture of 6-acetyl-8-cyclopentyl-5-methyl-2-[(5-piperazin-1-yl-2-pyridyl)amino]pyrido[2,3-d]pyrimidin-7-one (30 mg, 67 mol), DIPEA (43.3 mg, 335 mol) and (E)-4,4,4-trifluorobut-2-enoic acid (37.6 mg, 268 μmol) in DMSO (1 mL) was stirred at 90° C. for 16 h. After cooling down to rt, the reaction mixture was quenched with aq. HCl solution (5 mL, 2 N) and extracted with dichloromethane (3×10 mL). The combined organic phase was washed with brine, dried over Na₂SO₄, filtered and concentrated. The residue was purified by reverse-phase chromatography to provide the desired product (16 mg, 41% yield) as a yellow solid. MS (ESI) m/z=588.6 [M+H]⁺.

The remaining step was performed according to the standard procedure for preparing CPD-143 to provide the desired product (3.5 mg, 28% yield) as a yellow solid. MS (ESI) m/z=955.7 [M+H]⁺.

### Example 397. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)cyclobutyl)amino)-N-(6-(dimethylamino)pyridazin-3-yl)-6-((methyl-d₃)amino)nicotinamide (CPD-343)

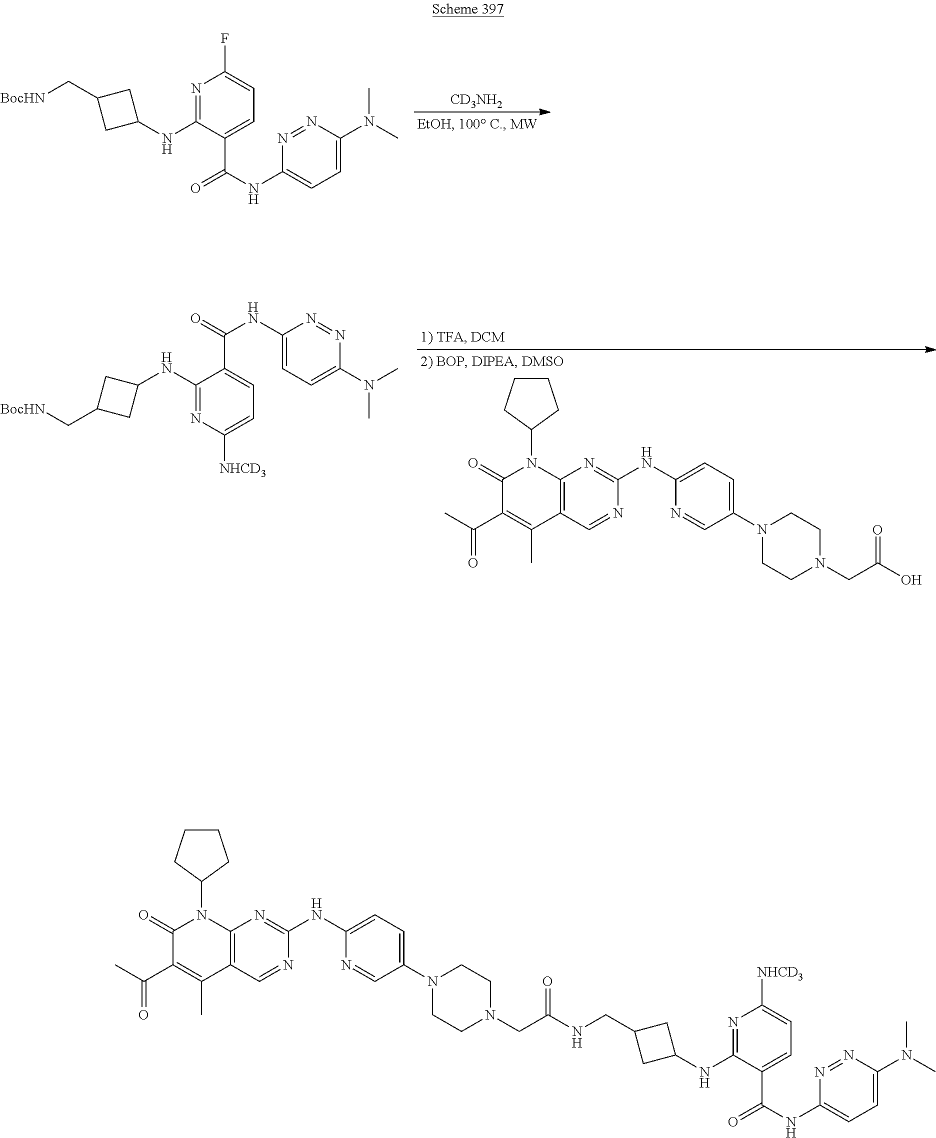

CPD-343 was synthesized following the standard procedures for preparing CPD-144 (10.0 mg, 26% yield over 3 steps) as a yellow solid. MS (ESI) m/z=861.7 [M+H]⁺.

### Example 398. 2-(((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-3-fluoroazetidin-3-yl)methyl)amino)-N-(6-(dimethylamino)pyridazin-3-yl)-6-(methylamino)nicotinamide (CPD-344)

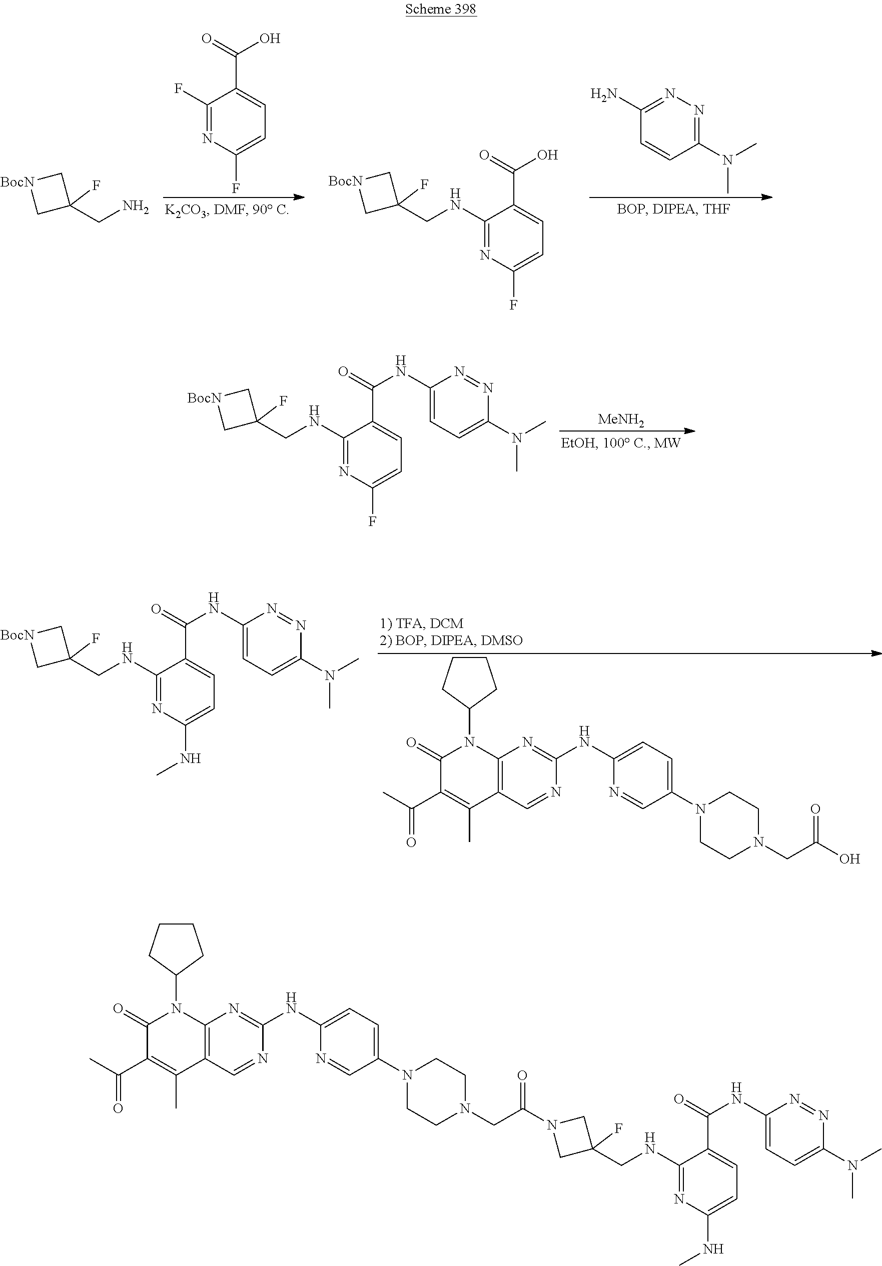

CPD-344 was synthesized following the standard procedures for preparing CPD-145 (12.0 mg, 7% yield over 5 steps) as a yellow solid. MS (ESI) m/z=862.6 [M+H]⁺.

### Example 399. 2-(((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)bicyclo[1.1.1]pentan-1-yl)methyl)amino)-N-(6-(dimethylamino)pyridazin-3-yl)-6-(methylamino)nicotinamide (CPD-345)

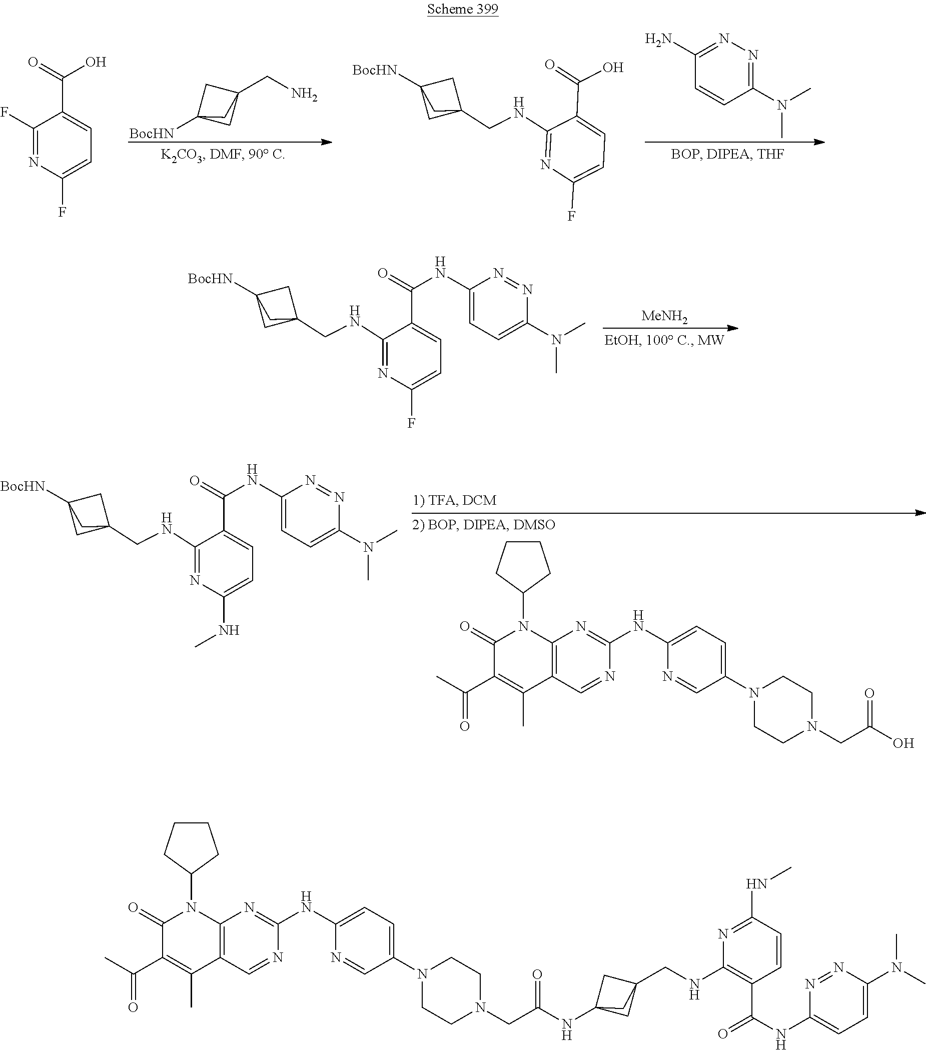

CPD-345 was synthesized following the standard procedures for preparing CPD-145 (15.0 mg, 9% yield over 5 steps) as a yellow solid. MS (ESI) m/z=870.7 [M+H]⁺.

### Example 400. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)bicyclo[1.1.1]pentan-1-yl)amino)-N-(6-(dimethylamino)pyridazin-3-yl)-6-(methylamino)nicotinamide (CPD-346)

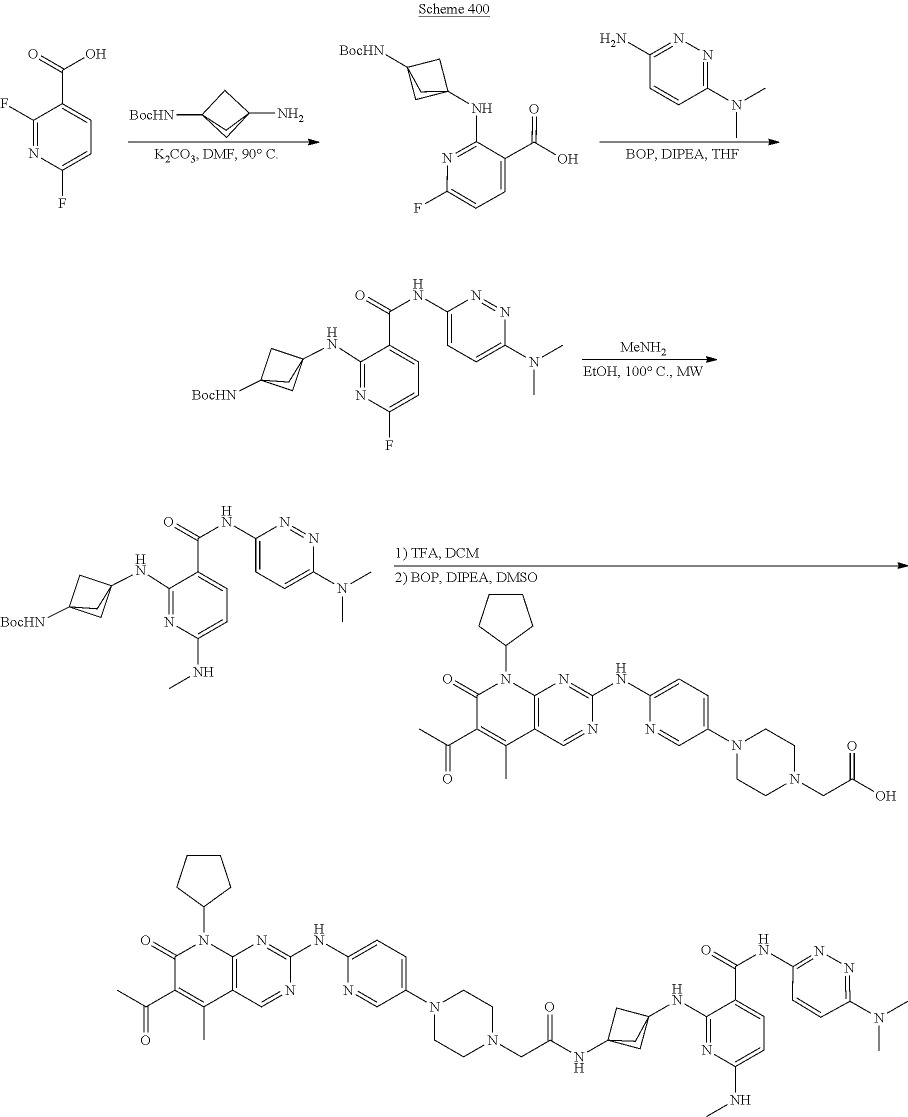

CPD-346 was synthesized following the standard procedures for preparing CPD-145 (8.0 mg, 5% yield over 5 steps) as a yellow solid. MS (ESI) m/z=856.7 [M+H]⁺.

### Example 401. 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.5]nonan-7-yl)amino)-N-(2-cyclopropyl-1-methyl-1H-imidazol-4-yl)-6-(methylamino)nicotinamide (CPD-347)

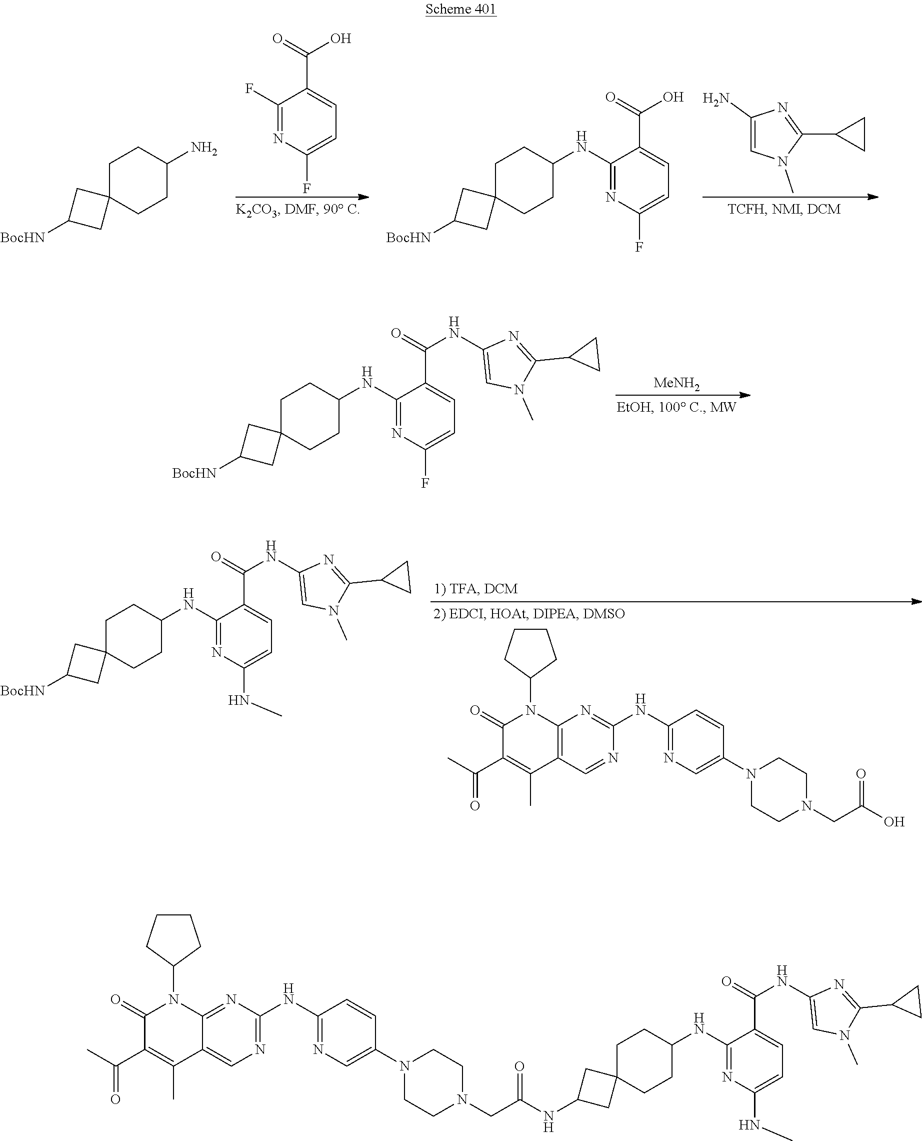

CPD-347 was synthesized following the standard procedures for preparing CPD-145 (5.3 mg, 4% yield over 5 steps) as a yellow solid. MS (ESI) m/z=911.7 [M+H]⁺.

### Example 402. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclobutane-1-carbonyl)-2-azaspiro[3.3]heptan-6-yl)amino)-N-(6-(dimethylamino)pyridazin-3-yl)-6-(methylamino)nicotinamide (CPD-348)

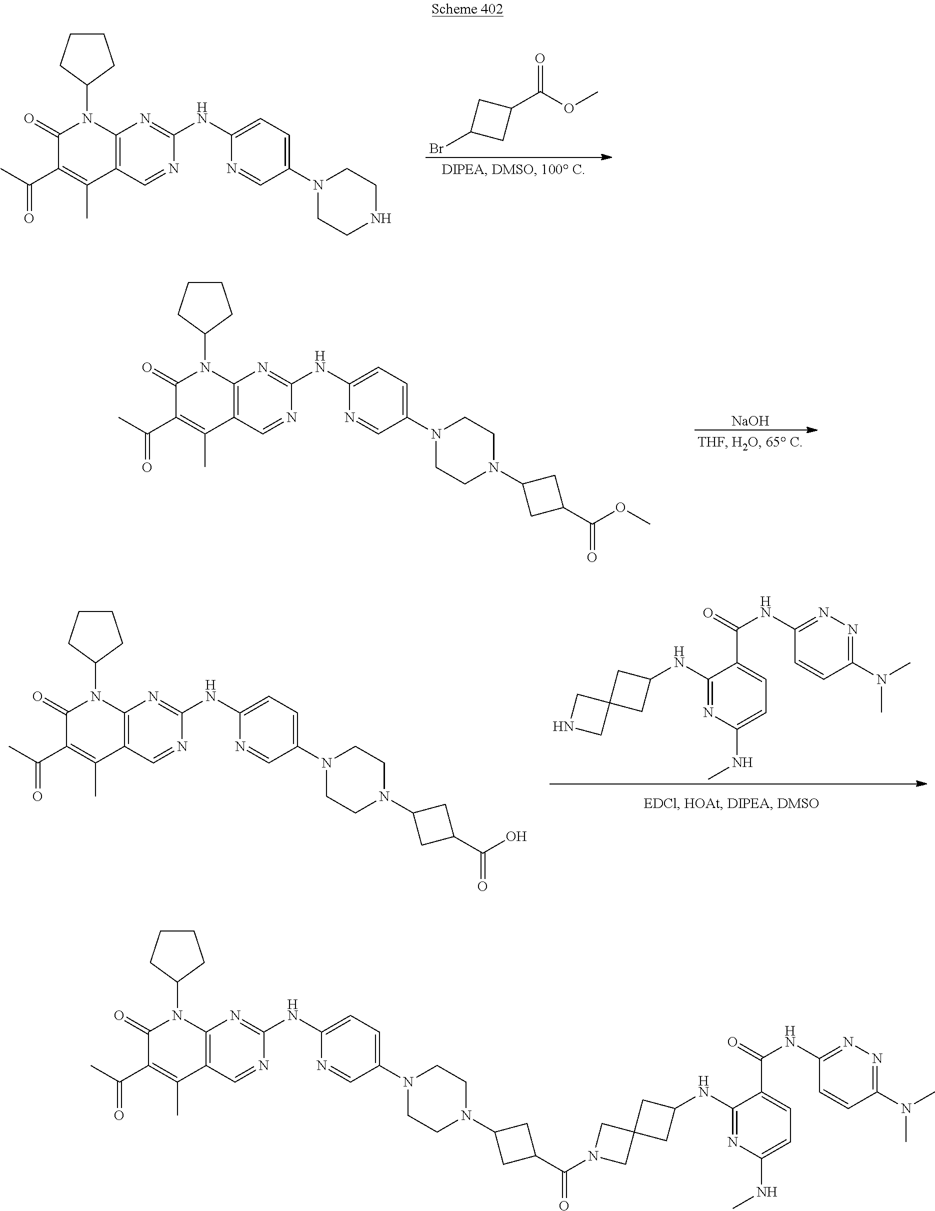

### Step 1. Synthesis of methyl 3-(4-(6-((6-Acetyl-8-cyclopentyl-5-methyl-7-oxo-7,8-dihydropyrido[2,3-d]pyrimidin-2-yl)amino)pyridin-3-yl)piperazin-1-yl)cyclobutane-1-carboxylate

A mixture of 6-acetyl-8-cyclopentyl-5-methyl-2-((5-(piperazin-1-yl)pyridin-2-yl)amino)pyrido[2,3-d]pyrimidin-7(8H)-one (60 mg, 0.13 mmol), methyl 3-bromocyclobutene carboxylate (46.6 mg, 0.24 mmol) and DIPEA (52 mg, 0.4 mmol) in DMSO (2 mL) was stirred at 100° C. for 12 h. The reaction mixture was cooled to rt and poured into ice water (10 mL). The mixture was extracted with dichloromethane (3×15 mL). The combined organic phase was washed with brine, dried over Na₂SO₄, filtered and concentrated. The residue was purified by reverse-phase chromatography to provide the desired product (15 mg, 20% yield) as a dark-brown solid. MS (ESI) m/z=560.5 [M+H]⁺.

### Step 2. Synthesis of 3-(4-(6-((6-acetyl-8-cyclopentyl-5-methyl-7-oxo-7,8-dihydro pyrido[2,3-d]pyrimidin-2-yl)amino)pyridin-3-yl)piperazin-1-yl)cyclobutane-1-carboxylic acid

A mixture of methyl 3-(4-(6-((6-acetyl-8-cyclopentyl-5-methyl-7-oxo-7,8-dihydro pyrido[2,3-d]pyrimidin-2-yl)amino)pyridin-3-yl)piperazin-1-yl)cyclobutane-1-carboxylate (15 mg, 26.8 μmol) and NaOH (10.7 mg, 0.27 mmol) in THF (1 mL) and water (1 mL) was stirred at 65° C. for 3 h. The mixture was cooled to rt and the pH was adjusted to 2 with aq. HCl solution (4 mL, 2 N). The aqueous phase was extracted with dichloromethane (3×10 mL). The combined organic phase was washed with brine, dried over Na₂SO₄, filtered and concentrated. The residue was diluted with EtOAc (10 mL) and filtered to provide the desired product (5 mg, 34% yield) as a yellow solid. This compound was used directly in the next step without further purification. MS (ESI) m/z=546.5 [M+H]⁺.

The remaining step was performed according to the standard procedure for preparing CPD-143 to provide the desired product (1.2 mg, 30% yield) as a yellow solid. MS (ESI) m/z=910.7 [M+H]⁺.

### Example 403. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(1-hydroxyethyl)nicotinamide (CPD-349)

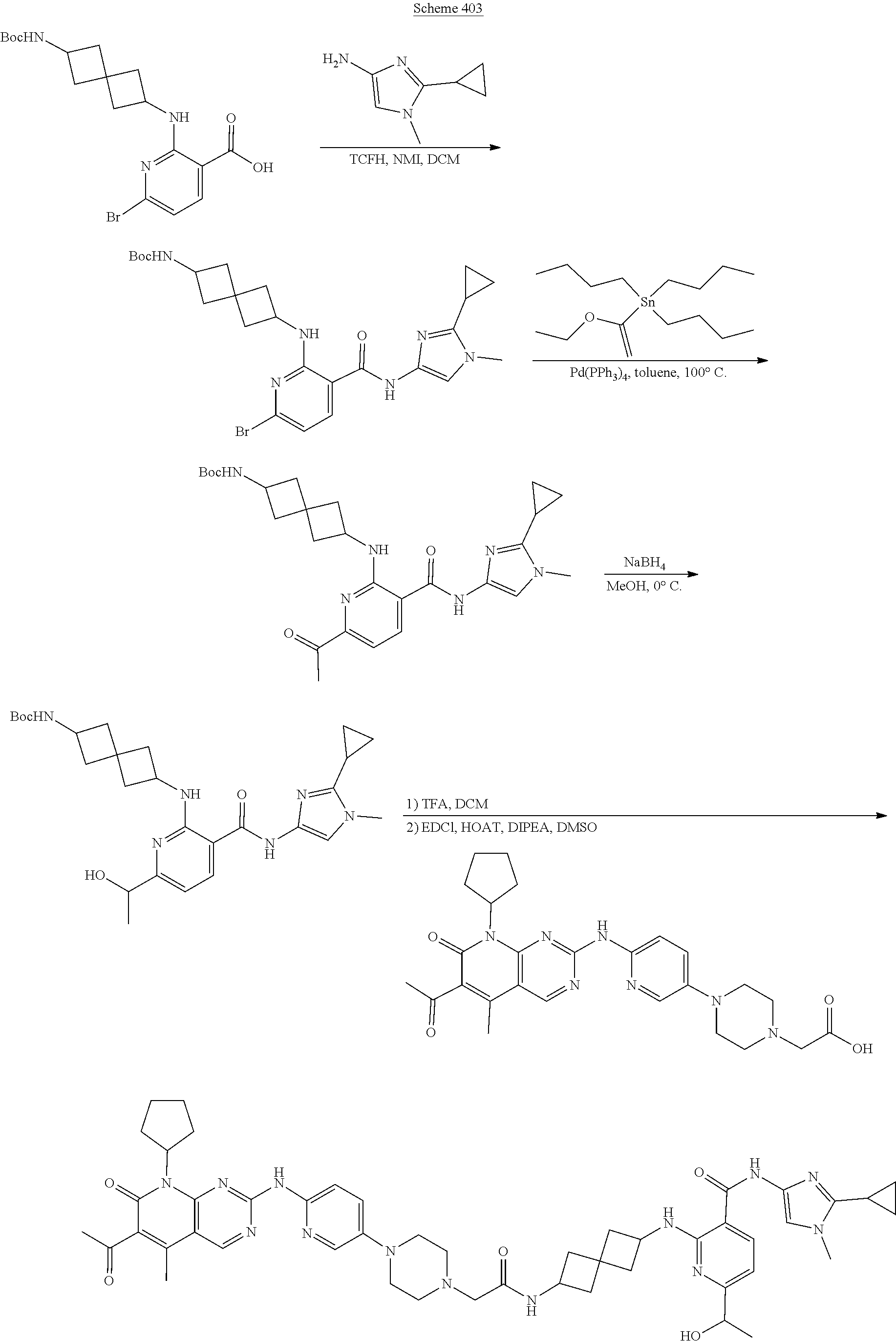

### Step 1. Synthesis of tert-butyl (6-((6-bromo-3-((2-cyclopropyl-1-methyl-1H-imidazol-4-yl)carbamoyl)pyridin-2-yl)amino)spiro[3.3]heptan-2-yl)carbamate

To a solution of 6-bromo-2-((6-((tert-butoxycarbonyl)amino)spiro[3.3]heptan-2-yl)amino)nicotinic acid (202 mg, 0.47 mmol), 2-cyclopropyl-1-methyl-1H-imidazol-4-amine (65 mg, 0.47 mmol) and TCFH (399 mg, 1.42 mmol) in dichloromethane (5 mL) was added NMI (0.1 mL). The reaction mixture was stirred at rt for 12 h. The mixture was quenched with H₂O (20 mL) and extracted with dichloromethane (20 mL×3). The combined organic layers were washed with brine and dried with anhydrous Na₂SO₄, filtered and concentrated. The resulting residue was purified by silica gel flash chromatography to give the desired product (130 mg, 50% yield) as a white solid. MS (ESI) m/z=547.2 [M+H]⁺.

### Step 2. Synthesis of tert-butyl (6-((6-acetyl-3-((2-cyclopropyl-1-methyl-1H-imidazol-4-yl)carbamoyl)pyridin-2-yl)amino)spiro[3.3]heptan-2-yl)carbamate

To a mixture of tert-butyl (6-((6-bromo-3-((2-cyclopropyl-1-methyl-1H-imidazol-4-yl)carbamoyl)pyridin-2-yl)amino)spiro[3.3]heptan-2-yl)carbamate (130 mg, 0.24 mmol), tributyl(1-ethoxyvinyl)stannane (172 mg, 0.48 mmol) in toluene (5 mL) was added Pd(PPh₃)₄ (28 mg, 0.024 mmol). The reaction mixture was stirred at 100 [symbol] for 12 h under N₂. The mixture was quenched with H₂O (20 mL) and extracted with ethyl acetate (20 mL×3). The combined organic layers were washed with brine and dried with anhydrous Na₂SO₄, filtered and concentrated. The resulting residue was purified by silica gel flash chromatography to give the desired product (50 mg, 41% yield) as a white solid. MS (ESI) m/z=509.7 [M+H]⁺.

### Step 3. Synthesis of tert-butyl (6-((3-((2-cyclopropyl-1-methyl-1H-imidazol-4-yl)carbamoyl)-6-(1-hydroxyethyl)pyridin-2-yl)amino)spiro[3.3]heptan-2-yl)carbamate

To a mixture of tert-butyl (6-((6-acetyl-3-((2-cyclopropyl-1-methyl-1H-imidazol-4-yl)carbamoyl)pyridin-2-yl)amino)spiro[3.3]heptan-2-yl)carbamate (10 mg, 0.02 mmol) in MeOH (1 mL) was added NaBH₄ (2 mg, 0.04 mmol). The reaction mixture was stirred at 0 [symbol] for 1 h. The mixture was quenched with H₂O (5 mL) and extracted with ethyl acetate (5 mL×3). The combined organic layers were washed with brine, dried with anhydrous Na₂SO₄, filtered, and concentrated to give the desired product (6 mg, 60% yield) as a colorless oil. The residue was used directly in the next step without further purification. MS (ESI) m/z=511.5 [M+H]⁺.

### Step 4. Synthesis of 2-((6-aminospiro[3.3]heptan-2-yl)amino)-N-(2-cyclopropyl-1-methyl-1H-imidazol-4-yl)-6-(1-hydroxyethyl)nicotinamide

To a mixture of tert-butyl (6-((3-((2-cyclopropyl-1-methyl-1H-imidazol-4-yl)carbamoyl)-6-(1-hydroxyethyl)pyridin-2-yl)amino)spiro[3.3]heptan-2-yl)carbamate (6 mg, 0.01 mmol) in dichloromethane (2 mL) was added TFA (0.2 mL). The reaction mixture was stirred at rt for 1 h. The mixture was concentrated to give the crude product (4 mg, 83% yield) as a yellow oil. The residue was used directly in the next step without further purification. MS (ESI) m/z=411.4 [M+H]⁺.

### Step 5. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(1-hydroxyethyl)nicotinamide

To a mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (6 mg, 0.012 mmol), 2-((6-aminospiro[3.3]heptan-2-yl)amino)-N-(2-cyclopropyl-1-methyl-1H-imidazol-4-yl)-6-(1-hydroxyethyl)nicotinamide (4 mg, 0.01 mmol), EDCI (4 mg, 0.02 mmol) and HOAT (3 mg, 0.02 mmol) in DMSO (2 mL) was added DIPEA (0.1 mL). The reaction mixture was stirred at rt for 12 h. The mixture was purified by pre-HPLC to give the desired product (2.7 mg, 30% yield) as a yellow solid. MS (ESI) m/z=898.6 [M+H]⁺.

### Example 404. 2-((4-(9-(6-((6-acetyl-8-cyclopentyl-5-methyl-7-oxo-7,8-dihydropyrido[2,3-d]pyrimidin-2-yl)amino)pyridin-3-yl)-3,9-diazaspiro[5.5]undecane-3-carbonyl)cyclohexyl)amino)-N-(6-(dimethylamino)pyridazin-3-yl)-6-(methylamino)nicotinamide (CPD-350)

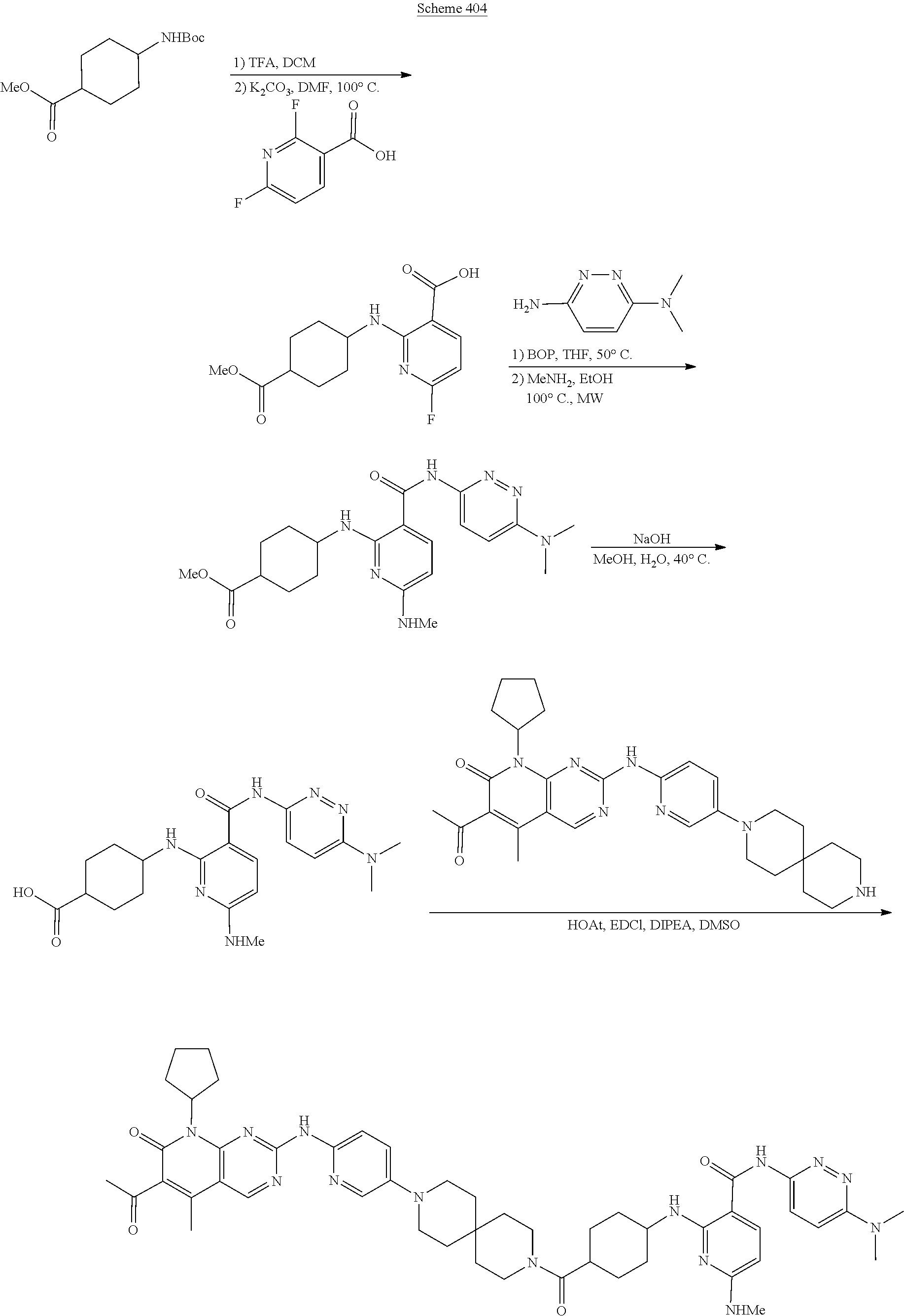

CPD-349 was synthesized following the standard procedures for preparing CPD-82 (1.6 mg, 2% yield over 6 steps) as a yellow solid. MS (ESI) m/z=911.8 [M+H]⁺.

### Example 405. 2-((4-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonyl)cyclohexyl)amino)-N-(6-(dimethylamino)pyridazin-3-yl)-6-(methylamino)nicotinamide (CPD-351)

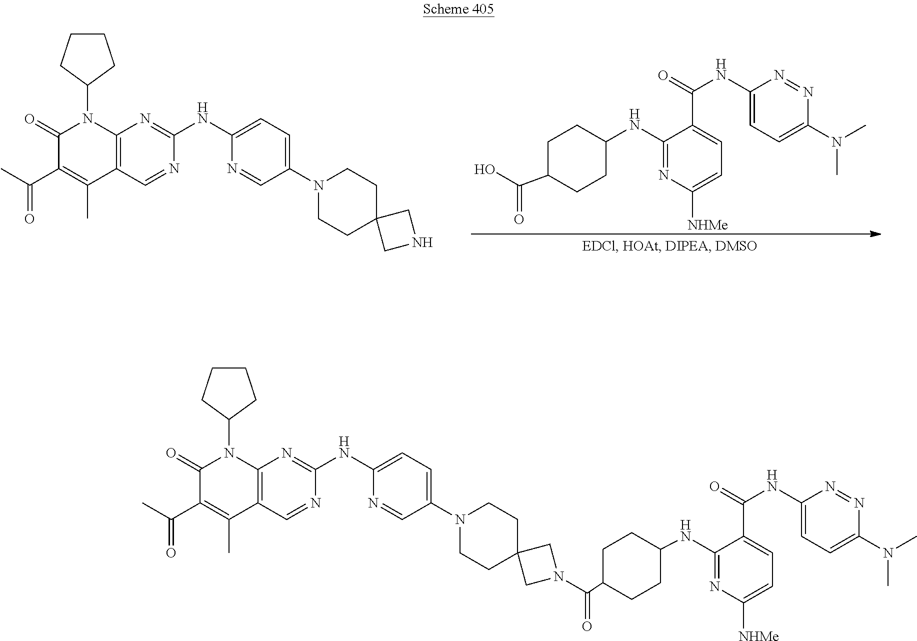 [SMILES: CC(=O)c1c(C)c2cnc(Nc3ccc(N4CCC5(CC4)CNC5)cn3)nc2n(C2CCCC2)c1=O ; CNc1ccc(C(=O)Nc2ccc(N(C)C)nn2)c(NC2CCC(C(=O)O)CC2)n1 ; CNc1ccc(C(=O)Nc2ccc(N(C)C)nn2)c(NC2CCC(C(=O)N3CC4(CCN(c5ccc(Nc6ncc7c(C)c(C(C)=O)c(=O)n(C8CCCC8)c7n6)nc5)CC4)C3)CC2)n1]

CPD-350 was synthesized following the standard procedure for preparing CPD-143 (1.6 mg, 11% yield) as a yellow solid. MS (ESI) m/z=883.7 [M+H]⁺.

### Example 406. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(2-hydroxypropan-2-yl)nicotinamide (CPD-352)

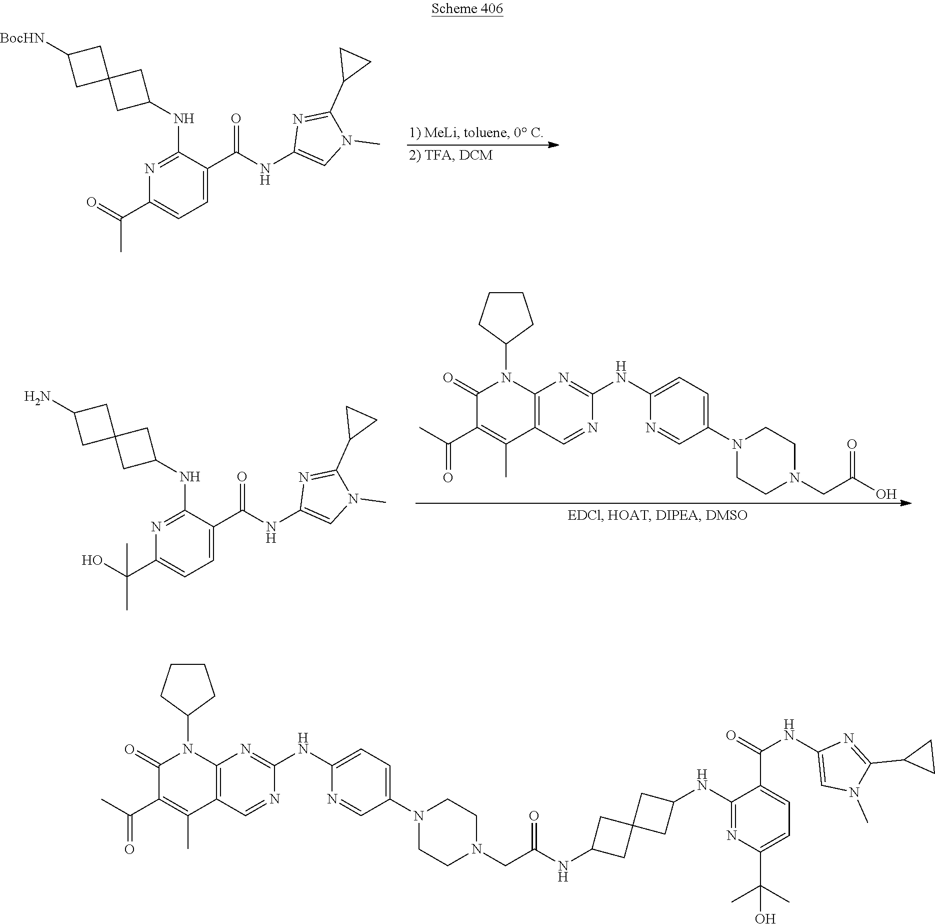

### Step 1. Synthesis of tert-butyl (6-((3-((2-cyclopropyl-1-methyl-1H-imidazol-4-yl)carbamoyl)-6-(2-hydroxypropan-2-yl)pyridin-2-yl)amino)spiro[3.3]heptan-2-yl)carbamate

To a solution of tert-butyl (6-((6-acetyl-3-((2-cyclopropyl-1-methyl-1H-imidazol-4-yl)carbamoyl)pyridin-2-yl)amino)spiro[3.3]heptan-2-yl)carbamate (20 mg, 0.04 mmol) in toluene (1 mL) was added MeLi (1.6 M, 0.05 mL). The reaction mixture was stirred at 0 [symbol] for 1 h. The mixture was quenched with H₂O (10 mL) and extracted with dichloromethane (10 mL×3). The combined organic layers were washed with brine and dried with anhydrous Na₂SO₄, filtered, and concentrated to give the desired product (10 mg, 48% yield) as a yellow oil. The residue was used directly in the next step without further purification. MS (ESI) m/z=525.5 [M+H]⁺.

### Step 2. Synthesis of 2-((6-aminospiro[3.3]heptan-2-yl)amino)-N-(2-cyclopropyl-1-methyl-1H-imidazol-4-yl)-6-(2-hydroxypropan-2-yl)nicotinamide

To a solution of tert-butyl (6-((3-((2-cyclopropyl-1-methyl-1H-imidazol-4-yl)carbamoyl)-6-(2-hydroxypropan-2-yl)pyridin-2-yl)amino)spiro[3.3]heptan-2-yl)carbamate (10 mg, 0.019 mmol) in dichloromethane (1 mL) was added TFA (0.2 mL). The reaction mixture was stirred at rt for 1 h. The mixture was concentrated to give the crude product (8 mg, 99% yield) as a yellow oil. The residue was used directly in the next step without further purification. MS (ESI) m/z=425.4 [M+H]⁺.

### Step 3. Synthesis of 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(2-cyclopropyl-1-methyl-1H-imidazol-4-yl)-6-(2-hydroxypropan-2-yl)nicotinamide

To a mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (17 mg, 0.034 mmol), 2-((6-aminospiro[3.3]heptan-2-yl)amino)-N-(2-cyclopropyl-1-methyl-1H-imidazol-4-yl)-6-(2-hydroxypropan-2-yl)nicotinamide (12 mg, 0.028 mmol), EDCI (11 mg, 0.056 mmol) and HOAT (8 mg, 0.056 mmol) in DMSO (2 mL) was added DIPEA (0.1 mL). The reaction mixture was stirred at rt for 12 h. The mixture was purified by pre-HPLC to give the desired product (2.7 mg, 11% yield) as a yellow solid. MS (ESI) m/z=912.8 [M+H]⁺.

### Example 407. 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-N-(1,5-dimethyl-1H-pyrazol-3-yl)-6-((methyl-d3)amino)nicotinamide (CPD-353)

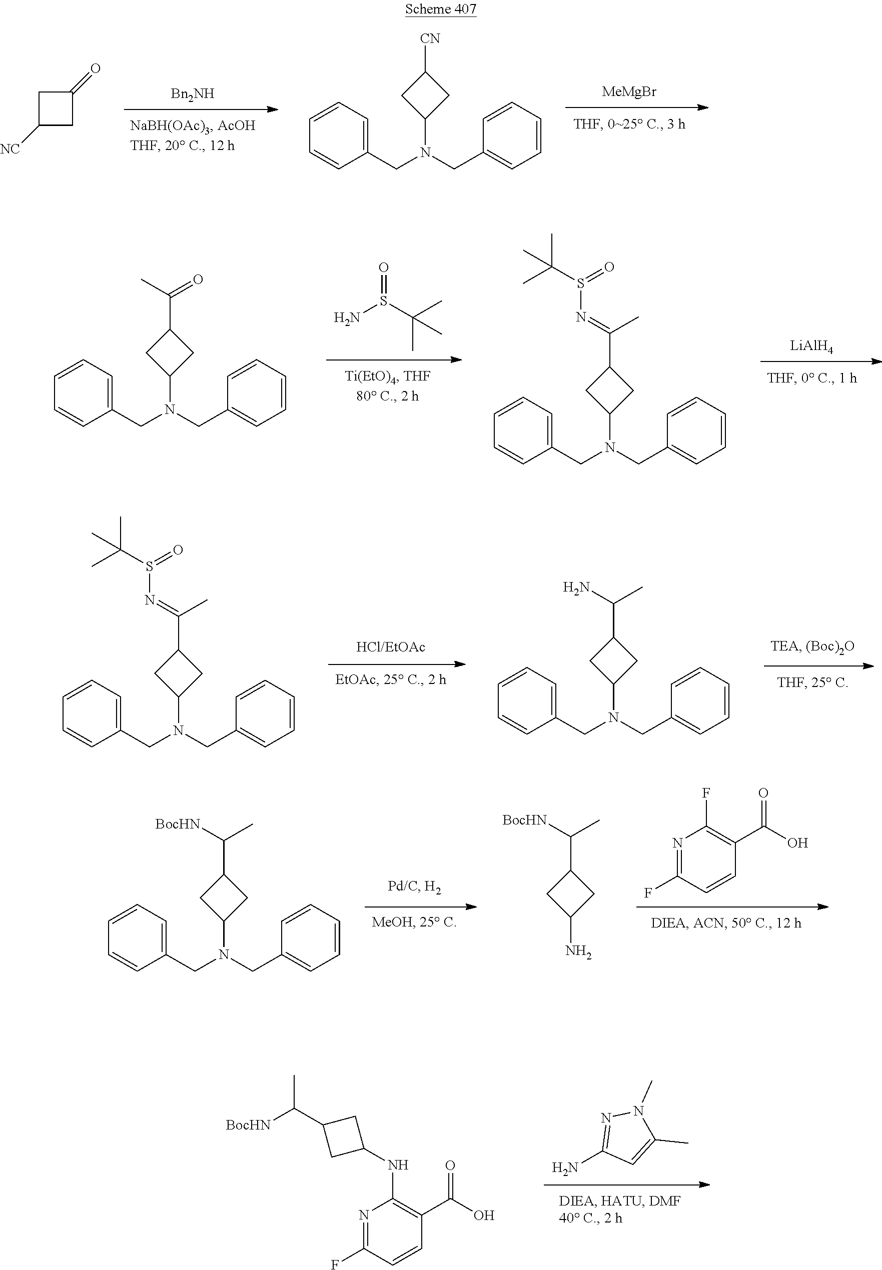

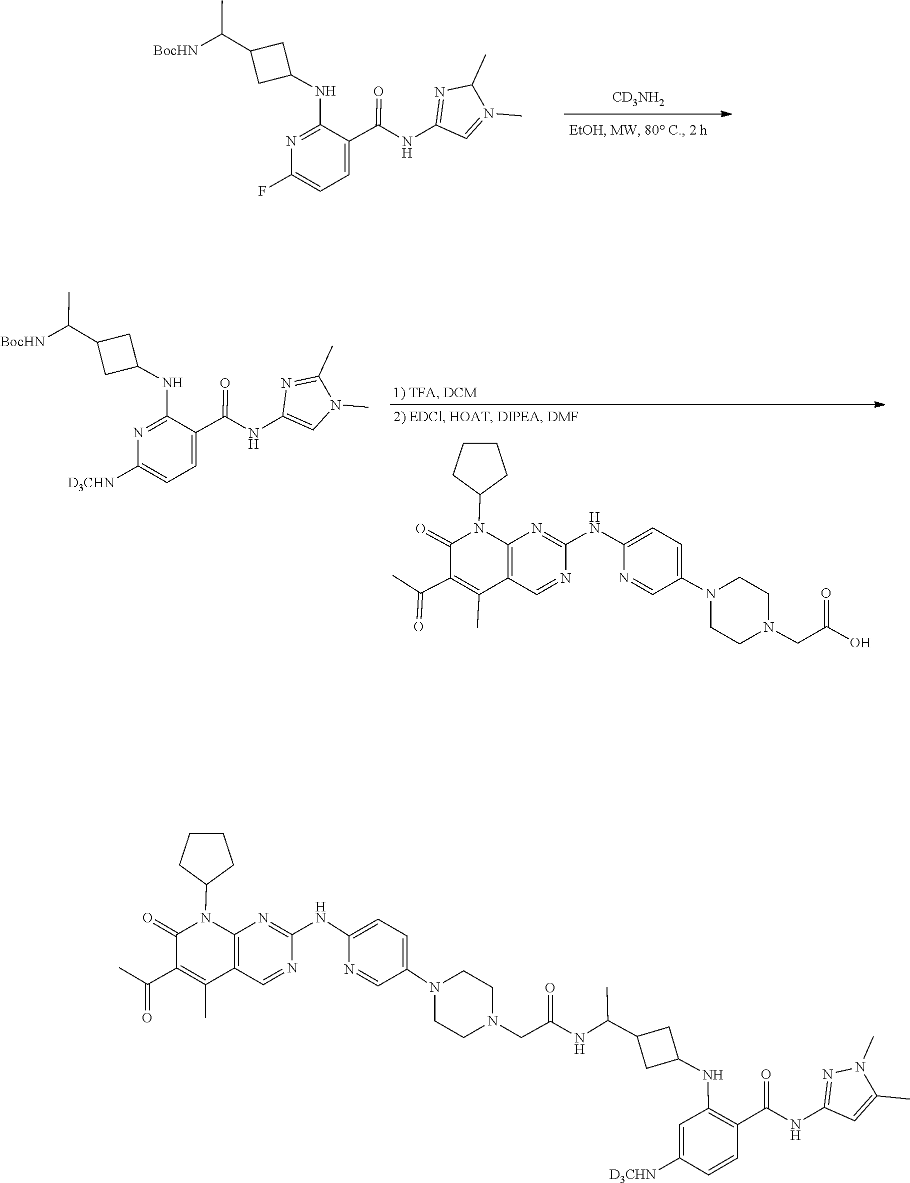

### Step 1. Synthesis of 3-(dibenzylamino)cyclobutane-1-carbonitrile

To a solution of 3-oxocyclobutanecarbonitrile (15.0 g, 157 mmol) and N-benzyl-1-phenyl-methanamine (46.6 g, 236 mmol) in dichloromethane (200 mL) were added NaBH(OAc) 3 (66.8 g, 315 mmol) and AcOH (4.74 g, 78.8 mmol). The mixture was stirred at 20° C. for 12 h. The reaction mixture was poured into saturated NH₄Cl aqueous solution (200 mL) and extracted with dichloromethane (50 mL×3). The combined organic layers were washed with brine (150 mL), dried over Na₂SO₄, filtered and concentrated. The residue was purified by column chromatography on silica gel (petroleum ether/ethyl acetate=50:1 to 10:1) to provide the desired product (12.0 g, 27% yield) as white solid. ¹HNMR (400 MHz, DMSO-d₆) δ 7.34-7.22 (m, 10H), 3.46 (s, 4H), 3.17-3.09 (m, 1H), 2.94-2.90 (m, 1H), 2.37-2.30 (m, 2H), 2.11-2.03 (m, 2H).

### Step 2. Synthesis of 1-(3-(dibenzylamino)cyclobutyl)ethan-1-one

To a solution of 3-(dibenzylamino)cyclobutanecarbonitrile (3.50 g, 12.6 mmol) in THF (35 mL) was added MeMgBr (3 M, 42.2 mL) at 0° C. The mixture was stirred at 25° C. for 3 h. The reaction mixture was poured into saturated NH₄Cl aqueous solution (500 mL) and extracted with ethyl acetate (100 mL×3). The combined organic layers were washed with brine (500 mL), dried over Na₂SO₄, filtered and concentrated. The residue was purified by column chromatography on silica (petroleum ether/ethyl acetate=50:1 to 10:1) to provide the desired product (2.00 g, 54% yield) as a white solid. ¹HNMR (400 MHz, CDCl₃) δ 7.19-7.17 (m, 8H), 7.13-7.10 (m, 2H), 3.37 (s, 4H), 3.04-2.99 (m, 1H), 2.49-2.47 (m, 1H), 2.21-2.19 (m, 11H), 2.11-2.07 (m, 2H), 1.98-1.92 (m, 2H).

### Step 3. Synthesis of (E)-N-(1-(3-(dibenzylamino)cyclobutyl)ethylidene)-2-methylpropane-2-sulfinamide

To a solution of 1-[3-(dibenzylamino)cyclobutyl]ethanone (1.20 g, 4.09 mmol) and 2-methylpropane-2-sulfinamide (743 mg, 6.13 mmol) in THF (10 mL) was added tetraethoxytitanium (1.87 g, 8.18 mmol). The mixture was stirred at 80° C. for 2 h. The reaction was concentrated. The residue was purified by column chromatography on silica gel (petroleum ether/ethyl acetate=5:1) to provide the desired product as a yellow oil (700 mg, 43% yield). ¹HNMR (400 MHz, CDCl₃) δ 7.31 (d, J=4.4 Hz, 7H), 7.31-7.27 (m, 7H), 7.25-7.23 (m, 3H), 3.51 (s, 4H), 3.16-3.12 (m, 1H), 2.74-2.69 (m, 1H), 2.28-2.21 (m, 5H), 2.12-1.98 (m, 2H), 1.25-1.23 (m, 9H).

### Step 4. Synthesis of N-(1-(3-(dibenzylamino)cyclobutyl)ethyl)-2-methylpropane-2-sulfinamide

To a solution of (E)-N-[1-[3-(dibenzylamino)cyclobutyl]ethylidene]-2-methyl-propane-2-sulfinamide (600 mg, 1.51 mmol) in THF (1 mL) was added LiAlH₄ (2.5 M, 726 μL) at 0° C. The mixture was stirred at 0° C. for 1 h. The reaction mixture was quenched with Na₂SO₄·10H₂O (2.00 g), filtered through a celite pad and evaporated in vacuo to provide the desired product as a yellow oil (500 mg, 83% yield). HNMR (400 MHz, CDCl₃) δ 7.35-7.22 (m, 101H), 3.54-3.43 (m, 4H), 3.25-3.21 (m, 1H), 3.04-2.82 (m, 2H), 2.26-2.06 (m, 2H), 1.88-1.82 (m, 1H), 1.74-1.67 (m, 1H), 1.63-1.55 (m, 4H), 1.20 (s, 9H), 1.17-1.07 (m, 3H).

### Step 5. Synthesis of 3-(1-aminoethyl)-N,N-dibenzylcyclobutan-1-amine

To a solution of N-[1-[3-(dibenzylamino)cyclobutyl]ethyl]-2-methyl-propane-2-sulfinamide (160 mg, 401 μmol) in EtOAc (2 mL) was added HCl solution (4 M in EtOAc, 2 mL). The mixture was stirred at 25° C. for 2 h. The reaction mixture was concentrated to provide the desired product as a white solid (130 mg, 97% yield, HCl salt). MS (ESI) m/z=295.0 [M+H]⁺.

### Step 6. Synthesis of tert-butyl (1-(3-(dibenzylamino)cyclobutyl)ethyl)carbamate

To a solution of 3-(1-aminoethyl)-N,N-dibenzyl-cyclobutanamine (130 mg, 392 μmol, HCl salt) in THF (1 mL) were added TEA (80 mg, 790 μmol) and (Boc)₂O (87 mg, 398 μmol). The mixture was stirred at 25° C. for 12 h. The reaction mixture was poured into H₂O (10 mL) and extracted with ethyl acetate (5 mL×3). The combined organic layers were washed with brine (10 mL), dried over Na₂SO₄, filtered and concentrated. The residue was purified by column chromatography on silica (petroleum ether/ethyl acetate=5:1) to provide the desired product as a white solid (150 mg, 96% yield). MS (ESI) m/z=395.2 [M+H]⁺.

### Step 7. Synthesis of tert-butyl (1-(3-aminocyclobutyl)ethyl)carbamate

A mixture of tert-butyl N-[1-[3-(dibenzylamino)cyclobutyl]ethyl]carbamate (150 mg, 380 μmol) and 10% Pd/C (404 mg) in MeOH (1 mL) was degassed and purged with N₂ for 3 times. Then the mixture was stirred at 25° C. for 12 h under H₂ atmosphere. The reaction mixture was filtered through a celite pad and the filtrate was evaporated in vacuo to provide the desired product as a white solid (80.0 mg, 98% yield). MS (ESI) m/z=159.2 [M-56+H]⁺.

### Step 8. Synthesis of 2-((3-(1-((tert-butoxycarbonyl)amino)ethyl)cyclobutyl)amino)-6-fluoronicotinic acid

To a solution of tert-butyl N-[1-(3-aminocyclobutyl)ethyl]carbamate (40.0 mg, 186 μmol) and 2,6-difluoropyridine-3-carboxylic acid (36.0 mg, 226 gmol) in MeCN (1 mL) was added DIEA (73.0 mg, 564 μmol). The mixture was stirred at 50° C. for 12 h. The reaction mixture was concentrated. The residue was purified by prep-TLC (petroleum ether/ethyl acetate=1:1) to provide the desired product as a white solid (45.0 mg, 36% yield). MS (ESI) m/z=237.1 [M-100+H]⁺.

### Step 9. Synthesis of tert-butyl (1-(3-((3-((1,5-dimethyl-1H-pyrazol-3-yl)carbamoyl)-6-fluoropyridin-2-yl)amino)cyclobutyl)ethyl)carbamate

To a solution of 2-[[3-[1-(2,2-dimethylpropanoylamino)ethyl]cyclobutyl]amino]-6-fluoro-pyridine-3-carboxylic acid (70.0 mg, 207 μmol) in DMF (1 mL) were added DIEA (80.0 mg, 618 μmol), HATU (96.0 mg, 252 μmol) and 1,5-dimethyl-1H-pyrazol-3-amine (28.0 mg, 251 μmol). The mixture was stirred at 40° C. for 2 h. The reaction mixture was poured into saturated NH₄Cl aqueous solution (10 mL) and extracted with ethyl acetate (10 mL×3). The combined organic layers were washed with brine (20 mL), dried over Na₂SO₄, filtered and concentrated. The residue was purified by prep-TLC (petroleum ether/ethyl acetate=1:1) to provide the desired product (25.0 mg, 27% yield) as a white solid. MS (ESI) m/z=447.2 [M+H]⁺.

### Step 10. Synthesis of tert-butyl (1-(3-((3-((1,5-dimethyl-1H-pyrazol-3-yl)carbamoyl)-6-((methyl-d3)amino)pyridin-2-yl)amino)cyclobutyl)ethyl)carbamate

To a solution of tert-butyl N-[1-[3-[[3-[(1,5-dimethylpyrazol-3-yl)carbamoyl]-6-fluoro-2-pyridyl]amino]cyclobutyl]ethyl]carbamate (25.0 mg, 55.9 μmol) and trideuterio-methanamine; hydrochloride (44.0 mg, 623 μmol) in EtOH (2 mL) was added DIEA (74.2 mg, 574 μmol).

The mixture was stirred at 80° C. for 2 h. The reaction mixture was concentrated and purified by prep-HPLC to provide the desired product as a white solid (15.0 mg, 49% yield). ¹HNMR (400 MHz, CDCl₃) δ 8.72-8.55 (m, 1H), 8.02 (s, 1H), 7.46 (d, J=8.0 Hz, 1H), 6.48 (s, 1H), 5.63 (d, J=8.4 Hz, 1H), 4.67-4.29 (m, 2H), 3.68 (s, 3H), 3.62-3.60 (m, 1H), 2.49-2.43 (m, 1H), 2.27 (s, 3H), 1.99-1.94 (m, 1H), 1.77-1.60 (m, 4H), 1.45 (s, 9H), 1.05 (d, J=6.4 Hz, 3H). MS (ESI) m/z=461.3 [M+H]⁺.

The remaining steps were performed according to the procedures for preparing CPD-144 to provide the desired product (1.6 mg, 16% yield for 2 steps) as a yellow solid. MS (ESI) m/z=848.8 [M+H]⁺.

### Example 408. 2-(((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)-5-oxa-2-azaspiro[3.4]octan-7-yl)methyl)amino)-N-(6-(dimethylamino)pyridazin-3-yl)-6-(methylamino)nicotinamide (CPD-354)

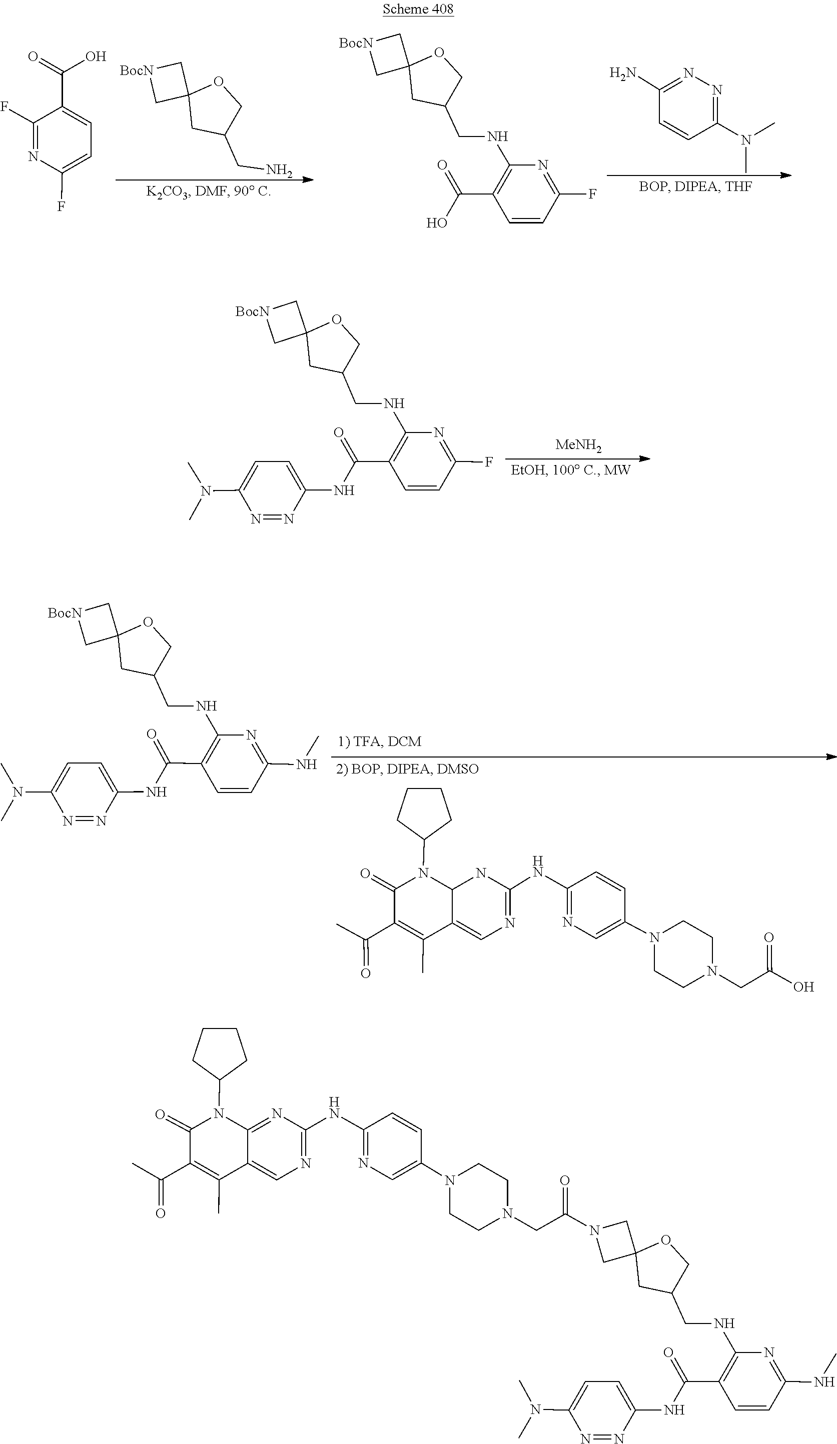

CPD-354 was synthesized following the standard procedures for preparing CPD-145 (18 mg, 12% yield over 5 steps) as a yellow solid. MS (ESI) m/z=900.6 [M+H]⁺.

### Example 409. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)azetidin-3-yl)amino)-N-(6-(dimethylamino)pyridazin-3-yl)-6-(isopropylamino)nicotinamide (CPD-355)

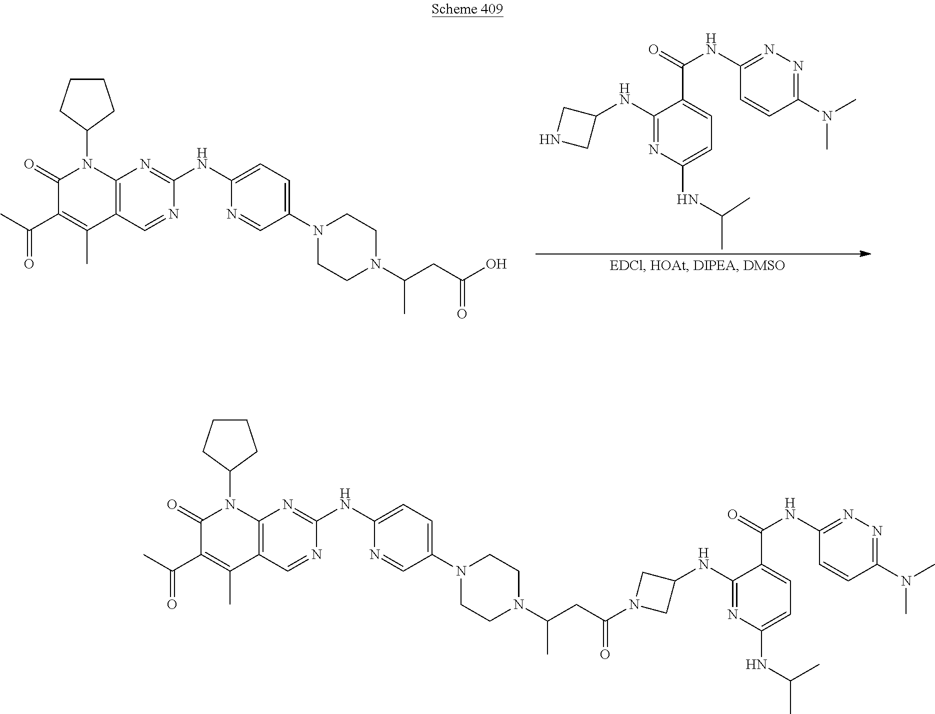 [SMILES: CC(=O)c1c(C)c2cnc(Nc3ccc(N4CCN(C(C)CC(=O)O)CC4)cn3)nc2n(C2CCCC2)c1=O ; CC(C)Nc1ccc(C(=O)Nc2ccc(N(C)C)nn2)c(NC2CNC2)n1 ; CC(=O)c1c(C)c2cnc(Nc3ccc(N4CCN(C(C)CC(=O)N5CC(Nc6nc(NC(C)C)ccc6C(=O)Nc6ccc(N(C)C)nn6)C5)CC4)cn3)nc2n(C2CCCC2)c1=O]

CPD-355 was synthesized following the standard procedure for preparing CPD-143 (1.5 mg, 12% yield) as a yellow solid. MS (ESI) m/z=886.8 [M+H]⁺.

### Example 410. 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)azetidin-3-yl)amino)-N-(6-(dimethylamino)pyridazin-3-yl)-6-(isopropylamino)nicotinamide (CPD-356)

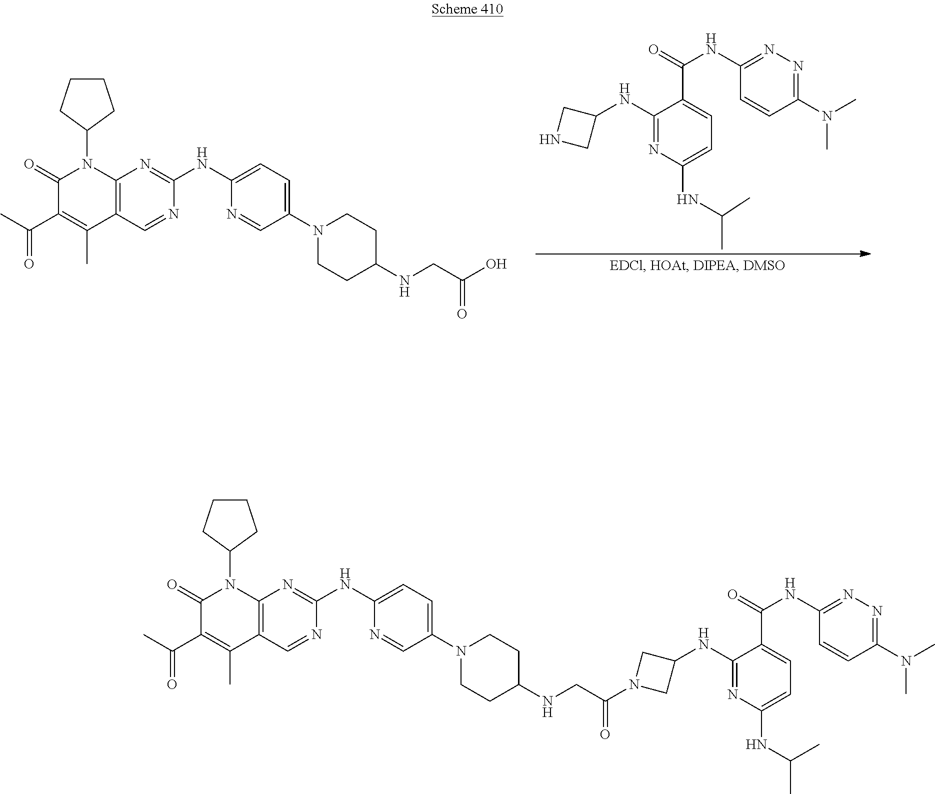 [SMILES: CC(=O)c1c(C)c2cnc(Nc3ccc(N4CCC(NCC(=O)O)CC4)cn3)nc2n(C2CCCC2)c1=O ; CC(C)Nc1ccc(C(=O)Nc2ccc(N(C)C)nn2)c(NC2CNC2)n1 ; CC(=O)c1c(C)c2cnc(Nc3ccc(N4CCC(NCC(=O)N5CC(Nc6nc(NC(C)C)ccc6C(=O)Nc6ccc(N(C)C)nn6)C5)CC4)cn3)nc2n(C2CCCC2)c1=O]

CPD-356 was synthesized following the standard procedure for preparing CPD-143 (3.0 mg, 16% yield) as a yellow solid. MS (ESI) m/z=872.7 [M+H]⁺.

### Example 411. 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)piperidin-4-yl)amino)-N-(6-(dimethylamino)pyridazin-3-yl)-6-(isopropylamino)nicotinamide (CPD-357)

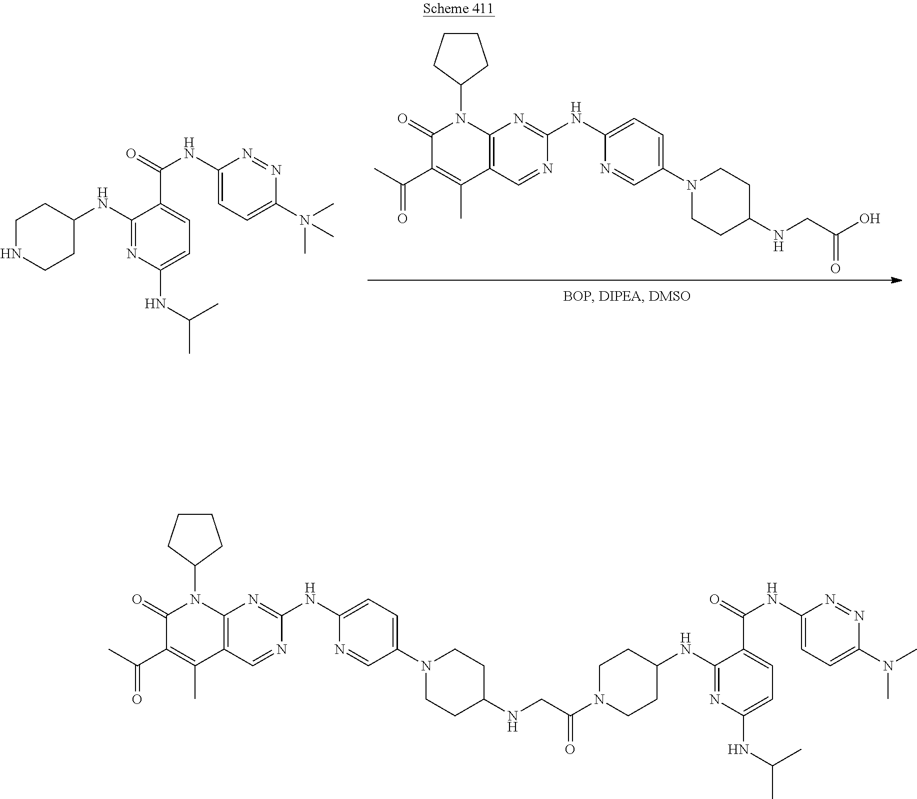

CPD-357 was synthesized following the standard procedure for preparing CPD-261 (6.0 mg, 27% yield) as a yellow solid. MS (ESI) m/z=900.7 [M+H]⁺.

### Example 412. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-(isopropylamino)nicotinamide (CPD-358)

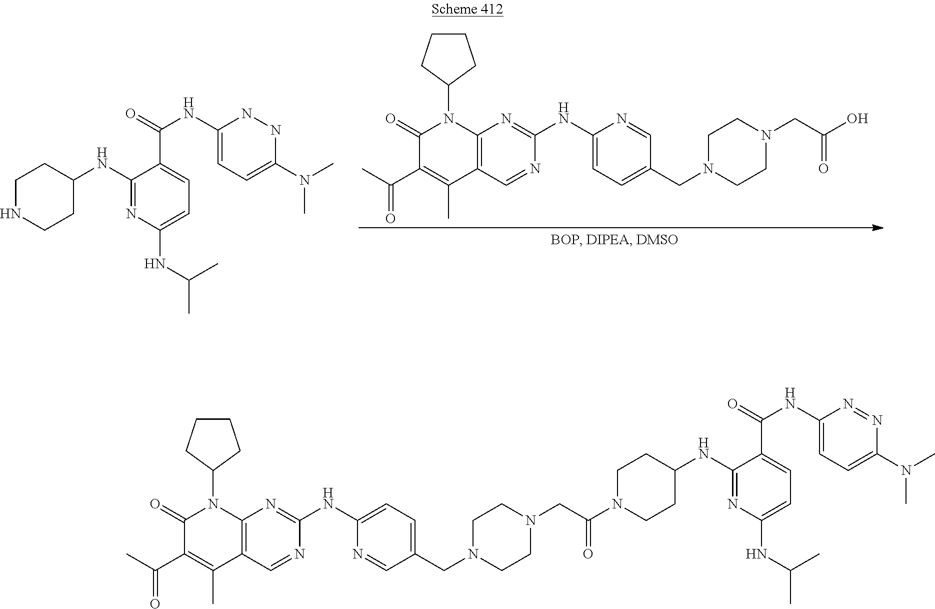

CPD-358 was synthesized following the standard procedure for preparing CPD-261 (8.0 mg, 35% yield) as a yellow solid. MS (ESI) m/z=900.7 [M+H]⁺.

### Example 413. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)piperidin-4-yl)amino)-N-(6-(dimethylamino)pyridazin-3-yl)-6-(isopropylamino)nicotinamide (CPD-359)

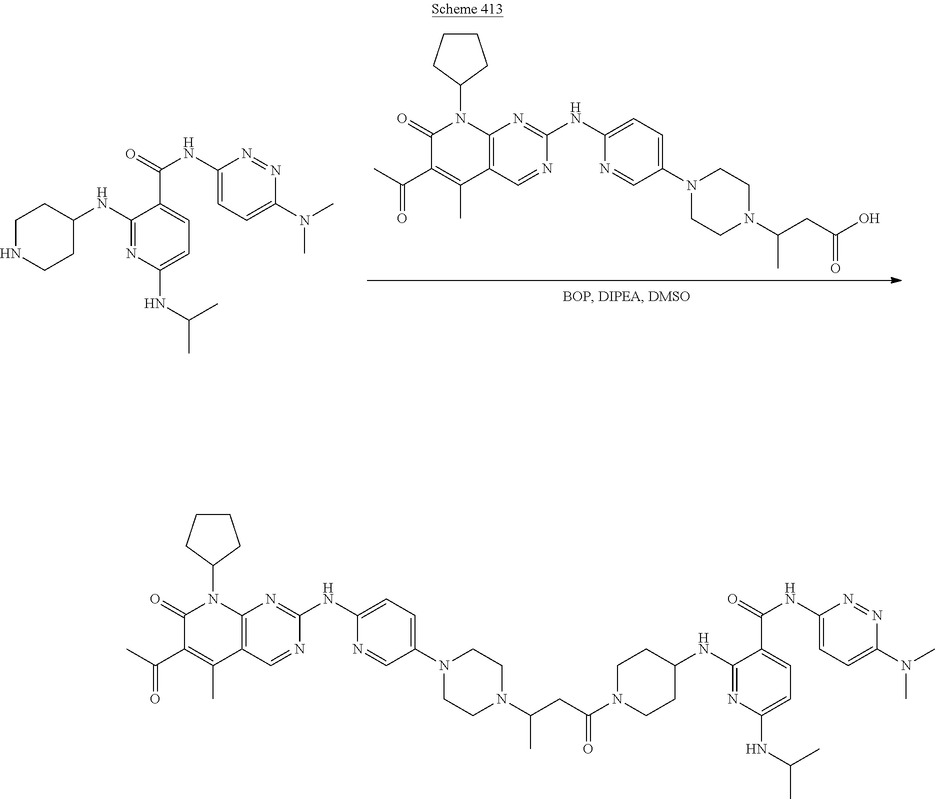 [SMILES: CC(C)Nc1ccc(C(=O)Nc2ccc(N(C)C)nn2)c(NC2CCNCC2)n1 ; CC(=O)c1c(C)c2cnc(Nc3ccc(N4CCN(C(C)CC(=O)O)CC4)cn3)nc2n(C2CCCC2)c1=O ; CC(=O)c1c(C)c2cnc(Nc3ccc(N4CCN(C(C)CC(=O)N5CCC(Nc6nc(NC(C)C)ccc6C(=O)Nc6ccc(N(C)C)nn6)CC5)CC4)cn3)nc2n(C2CCCC2)c1=O]

CPD-359 was synthesized following the standard procedure for preparing CPD-261 (6.0 mg, 26% yield) as a yellow solid. MS (ESI) m/z=914.7 [M+H]⁺.

### Example 414. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(6-(dimethylamino)pyridazin-3-yl)-6-(isopropylamino)nicotinamide (CPD-360)

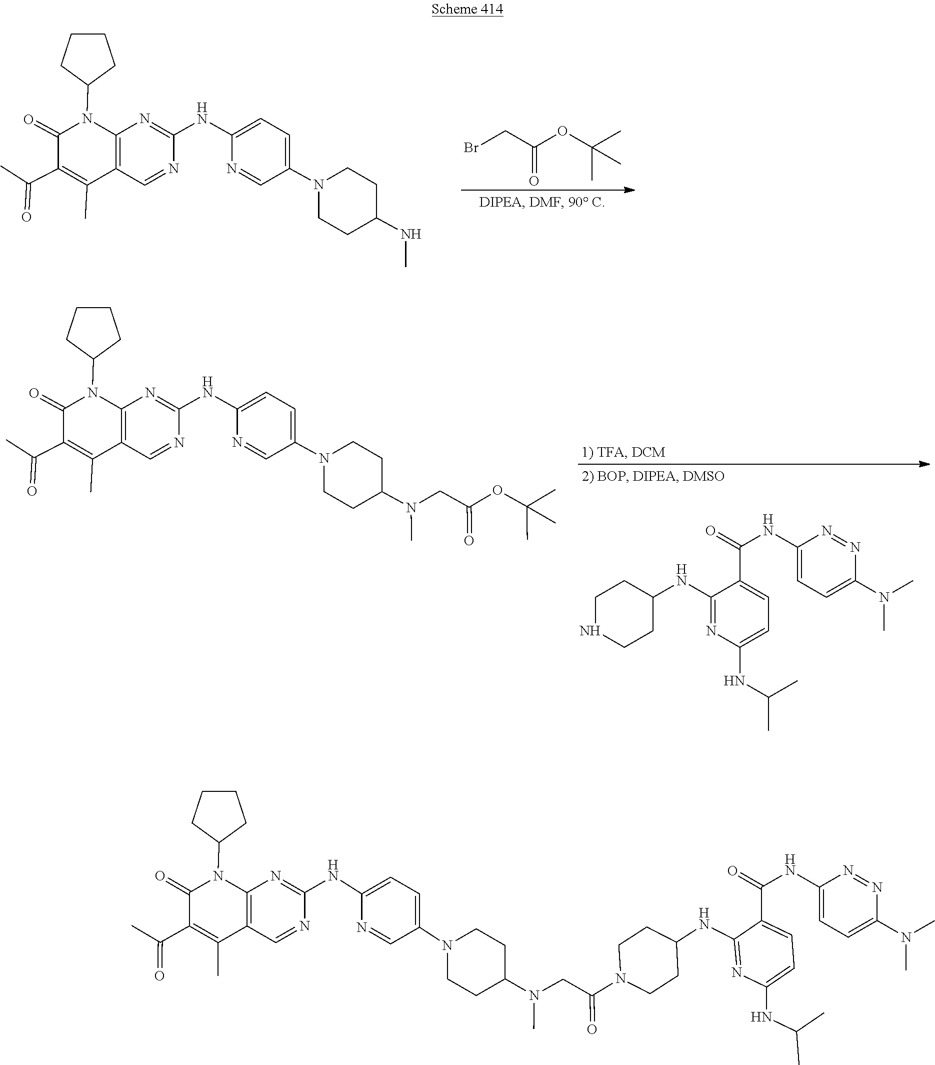

CPD-360 was synthesized following the standard procedures for preparing CPD-261 (10.0 mg, 34% yield over 3 steps) as a yellow solid. MS (ESI) m/z=914.6 [M+H]⁺.

### Example 417. 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-((methyl-d₃)amino)-N-(5-methylisoxazol-3-yl)nicotinamide (CPD-361)

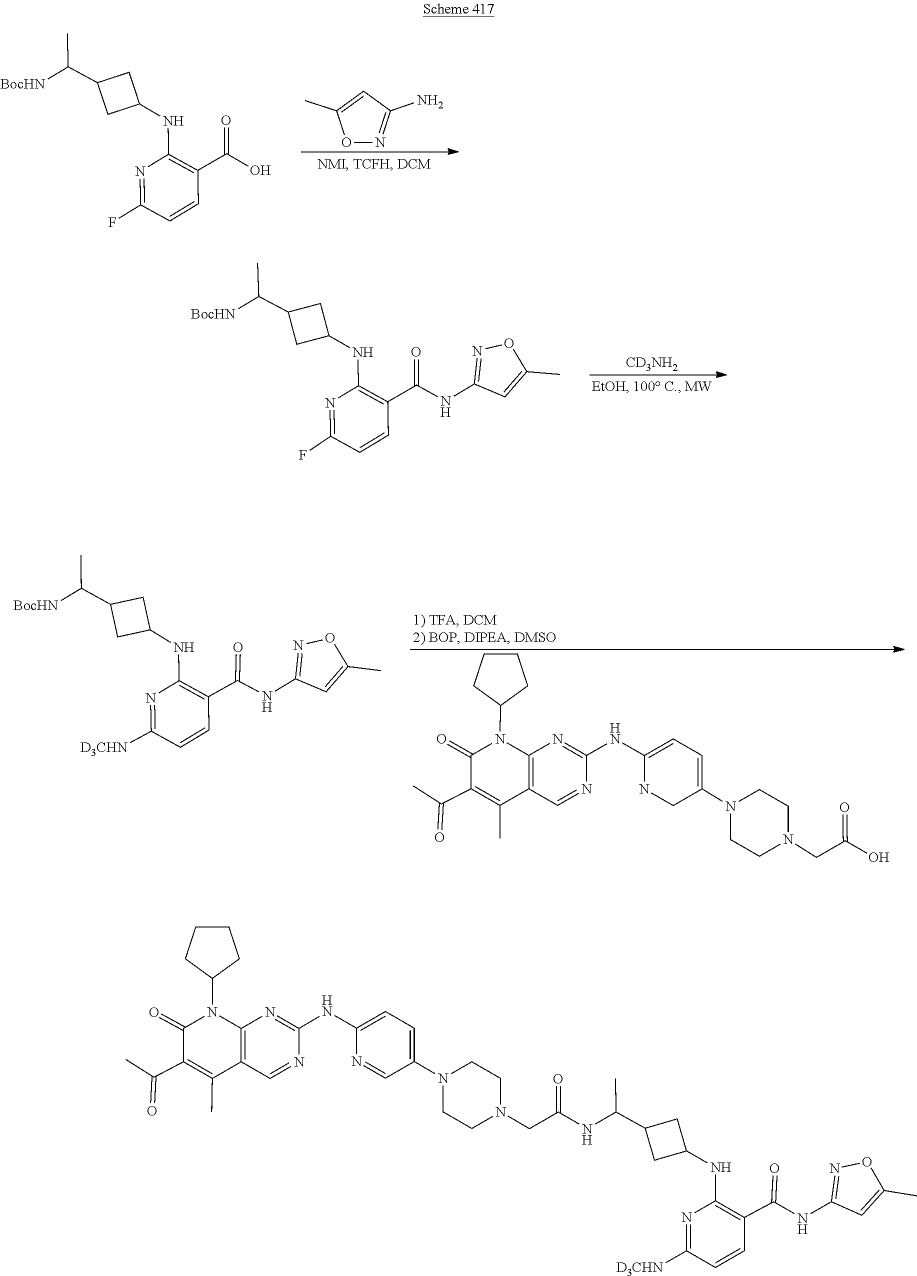

CPD-361 was synthesized following the standard procedures for preparing CPD-144 (2.0 mg, 1% yield over 4 steps) as a yellow solid. MS (ESI) m/z=835.7 [M+H]⁺.

### Example 418. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)spiro[3.3]heptan-2-yl)amino)-N-(6-(dimethylamino)pyridazin-3-yl)-6-(methylamino)nicotinamide (CPD-362)

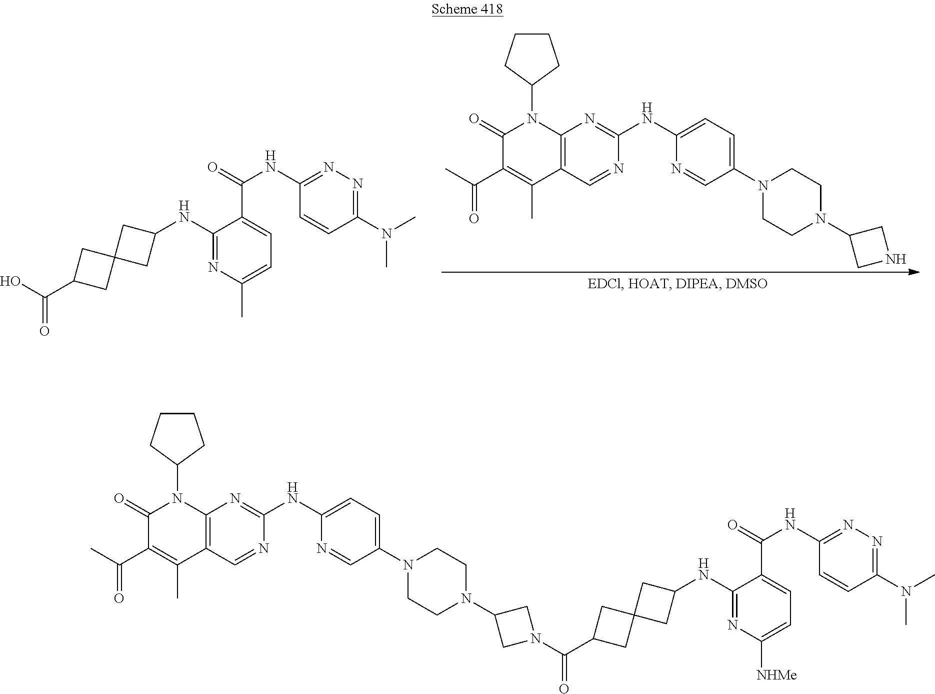 [SMILES: Cc1ccc(C(=O)Nc2ccc(N(C)C)nn2)c(NC2CC3(C2)CC(C(=O)O)C3)n1 ; CC(=O)c1c(C)c2cnc(Nc3ccc(N4CCN(C5CNC5)CC4)cn3)nc2n(C2CCCC2)c1=O ; CNc1ccc(C(=O)Nc2ccc(N(C)C)nn2)c(NC2CC3(C2)CC(C(=O)N2CC(N4CCN(c5ccc(Nc6ncc7c(C)c(C(C)=O)c(=O)n(C8CCCC8)c7n6)nc5)CC4)C2)C3)n1]

CPD-362 was synthesized following the standard procedure for preparing CPD-143 (3.6 mg, 14% yield) as a yellow solid. MS (ESI) m/z=910.7 [M+H]⁺.

### Example 419. 2-((6-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[3.5]nonane-2-carbonylspiro[3.3]heptan-2-yl)amino)-N-(6-(dimethylamino)pyridazin-3-yl)-6-(methylamino)nicotinamide (CPD-363)

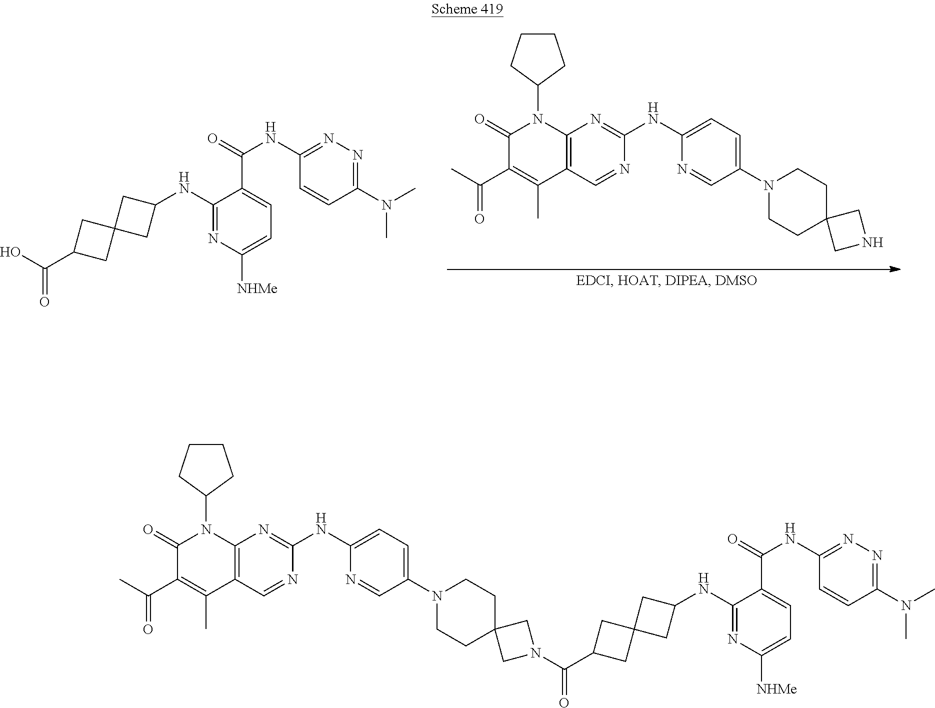

CPD-363 was synthesized following the standard procedure for preparing CPD-143 (4.1 mg, 17% yield) as a yellow solid. MS (ESI) m/z=895.6 [M+H]⁺.

### Example 420. 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)-2-azaspiro[3.3]heptan-6-yl)amino)-N-(6-(dimethylamino)pyridazin-3-yl)-6-(methylamino)nicotinamide (CPD-364)

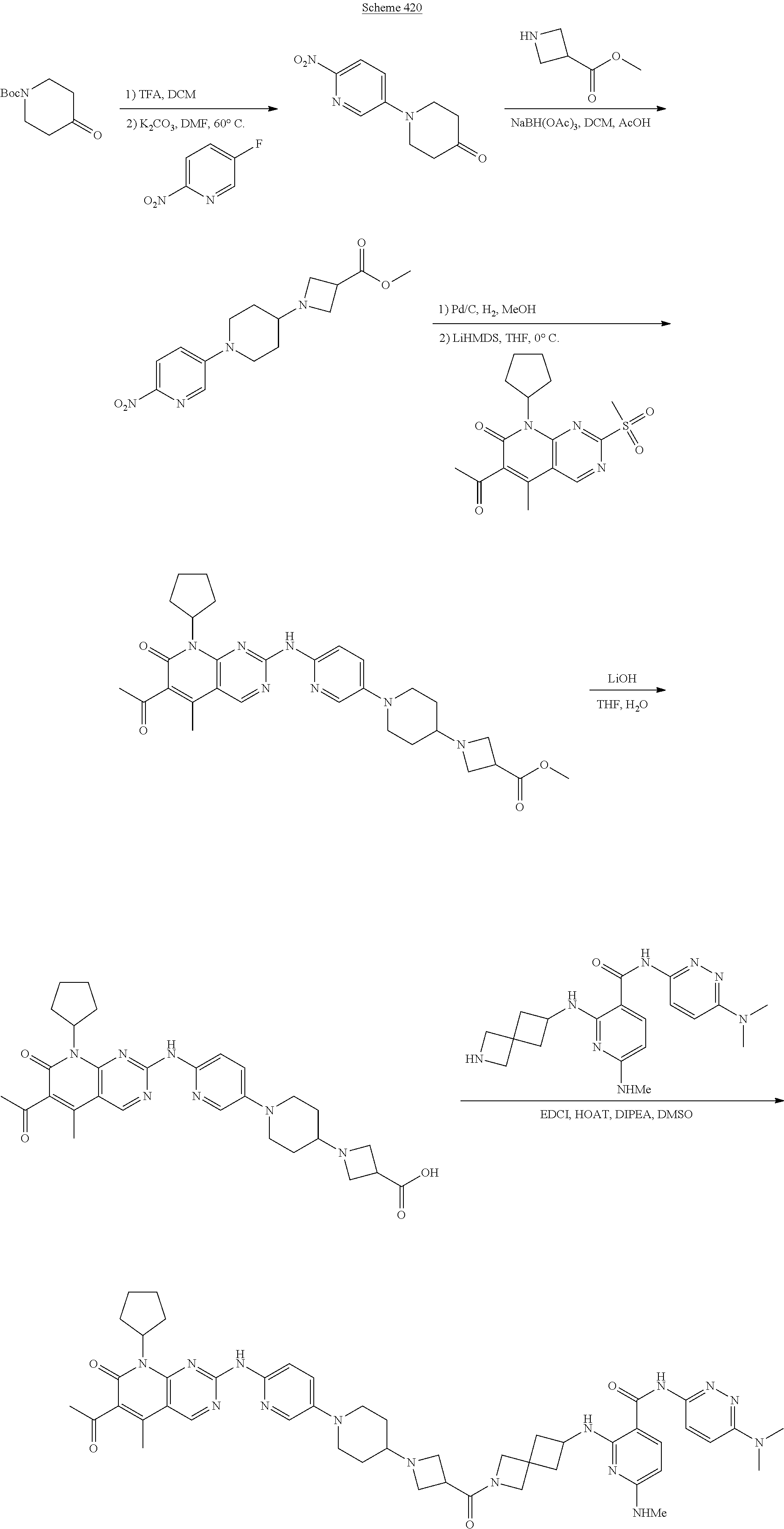

### Step 1. Synthesis of piperidin-4-one

To a solution of tert-butyl 4-oxopiperidine-1-carboxylate (1 g, 5.02 mmol) in dichloromethane (5 mL) was added TFA (1 mL). The mixture was stirred at rt for 1 h. The solution was concentrated to give the desired product (1 g, 93% yield) as a colorless oil. This compound was used directly in the next step without further purification.

### Step 2. Synthesis of 1-(6-nitropyridin-3-yl)piperidin-4-one

To a mixture of 5-fluoro-2-nitropyridine (666 mg, 4.69 mmol) and piperidin-4-one (1 g, 4.69 mmol) in DMF (10 mL) was added K₂CO₃ (1.9 g, 14 mmol) at room temperature. The reaction mixture was stirred at 60° C. for 16 h. The reaction mixture was poured into water (20 mL) and extracted with ethyl acetate (3×20 mL). The combined organic layers were washed with saturated brine (20 mL), dried over anhydrous Na₂SO₄, filtered and concentrated. The resulting residue was purified by silica gel flash chromatography to give the desired product (750 mg, 72% yield) as a white solid. MS (ESI) m/z=222.1 [M+H]⁺.

### Step 3. Synthesis of methyl 1-(1-(6-nitropyridin-3-yl)piperidin-4-yl)azetidine-3-carboxylate

To a solution of 1-(6-nitropyridin-3-yl)piperidin-4-one (70 mg, 0.3 mmol) in dichloromethane (10 mL) and AcOH (1 mL) were added methyl azetidine-3-carboxylate (96 mg, 0.6 mmol) and NaBH(OAc)₃ (134 mg, 0.6 mmol). The reaction mixture was stirred at rt for 2 h. The reaction mixture was poured into water (10 mL) and extracted with dichloromethane (3×10 mL). The combined organic layers were washed with saturated brine (10 mL), dried over anhydrous Na₂SO₄, filtered and concentrated. The resulting residue was purified by silica gel flash chromatography to give the desired product (98 mg, 78% yield) as a yellow solid. MS (ESI) m/z=321.3 [M+H]⁺.

### Step 4. Synthesis of methyl 1-(1-(6-aminopyridin-3-yl)piperidin-4-yl)azetidine-3-carboxylate

To a solution of methyl 1-(1-(6-nitropyridin-3-yl)piperidin-4-yl)azetidine-3-carboxylate (98 mg, 0.3 mmol) in MeOH (10 mL) was added 10% Pd/C (10 mg). The reaction mixture was stirred at rt for 16 h under hydrogen balloon. The reaction mixture was filtered and concentrated to give the desired product (32 mg, 79% yield) as a colorless oil. MS (ESI) m/z=291.2 [M+H]⁺.

### Step 5. Synthesis of methyl 1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxylate

To a mixture of methyl 1-(1-(6-aminopyridin-3-yl)piperidin-4-yl)azetidine-3-carboxylate (87 mg, 0.3 mmol) and 6-acetyl-8-cyclopentyl-5-methyl-2-(methylsulfonyl)pyrido[2,3-d]pyrimidin-7(8H)-one (70 mg, 0.2 mmol) in THF (5 mL) was added LiHMDS (0.4 mL, 1 M in THF) at 0° C. The reaction mixture was stirred at 0° C. for 2 h. The reaction mixture was quenched with water (15 mL) and extracted with ethyl acetate (3×20 mL). The organic layers were washed with saturated brine (10 mL), dried over anhydrous Na₂SO₄, filtered and concentrated. The resulting residue was purified by silica gel flash chromatography to give the desired product (45 mg, 40% yield) as a colorless oil. MS (ESI) m/z=560.6 [M+H]⁺.

### Step 6. Synthesis of 1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxylic acid

A mixture of methyl 1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxylate (45 mg, 0.08 mmol) and lithium hydroxide (22 mg, 0.9 mmol) in THF (2 mL) and H₂O (1 mL) was stirred at rt for 2 h. The reaction mixture was purified by reverse-phase chromatography to give the desired product (35 mg, 66% yield) as a white solid. MS (ESI) m/z=546.5 [M+H]⁺.

### Step 7. Synthesis of 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)-2-azaspiro[3.3]heptan-6-yl)amino)-N-(6-(dimethylamino)pyridazin-3-yl)-6-(methylamino)nicotinamide

A mixture of 1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxylic acid (5 mg, 0.01 mmol), 2-((2-azaspiro[3.3]heptan-6-yl)amino)-N-(6-(dimethylamino)pyridazin-3-yl)-6-(methylamino)nicotinamide (3.5 mg, 0.01 mmol), EDCI (6.7 mg, 0.03 mmol), HOAt (4.8 mg, 0.03 mmol) and DIPEA (16 mg, 0.1 mmol) in DMSO (2 mL) was stirred at rt for 16 h. The reaction mixture was purified by pre-HPLC to give the desired product (2.1 mg, 22% yield) as a yellow solid. MS (ESI) m/z=910.7 [M+H]⁺.

### Example 421. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanamido)cyclobutyl)amino)-N-(6-(dimethylamino)pyridazin-3-yl)-6-(isopropylamino)nicotinamide (CPD-365)

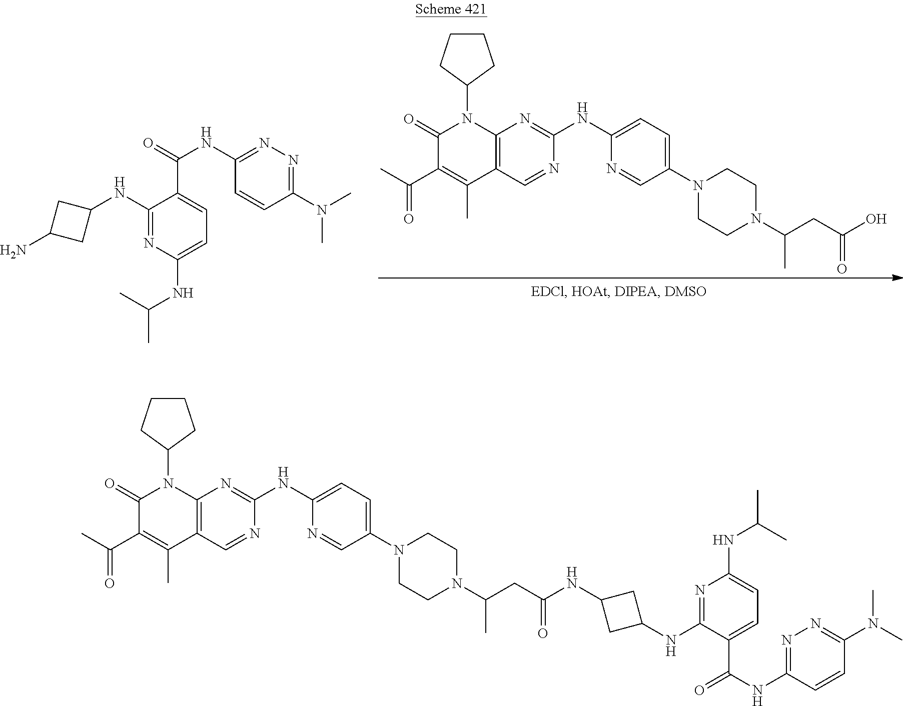

CPD-364 was synthesized following the standard procedure for preparing CPD-143 (5.0 mg, 21% yield) as a yellow solid. MS (ESI) m/z=900.7 [M+H]⁺.

### Example 422. 2-((6-(1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclopropane-1-carboxamido)spiro[3.3]heptan-2-yl)amino)-N-(2-cyclopropyl-1-methyl-1H-imidazol-4-yl)-6-(isopropylamino)nicotinamide (CPD-366)

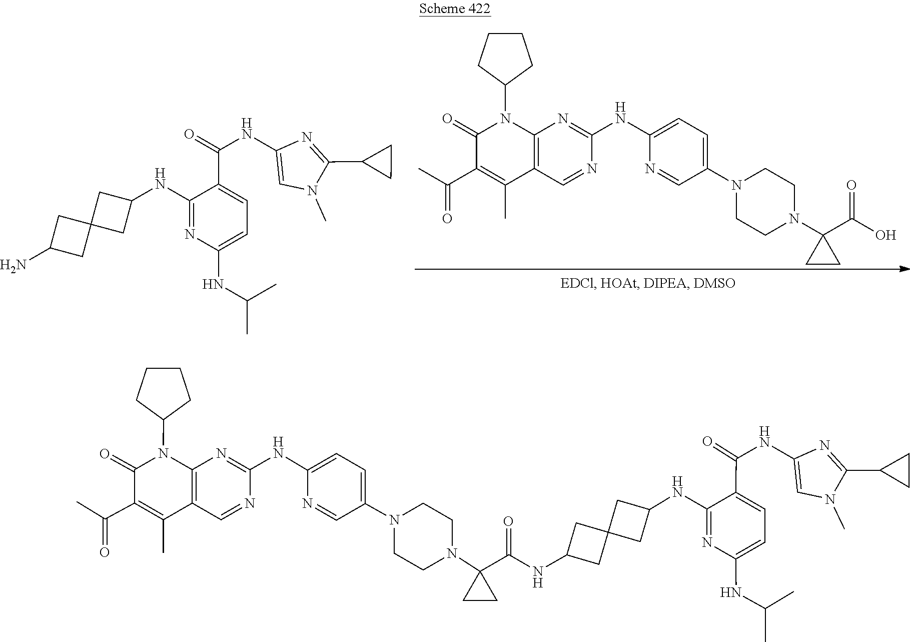

CPD-366 was synthesized following the standard procedure for preparing CPD-143 (7.1 mg, 40% yield) as a yellow solid. MS (ESI) m/z=937.7 [M+H]⁺.

### Example 423. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-(dimethylamino)pyridazin-3-yl)-6-(isopropylamino)nicotinamide (CPD-367)

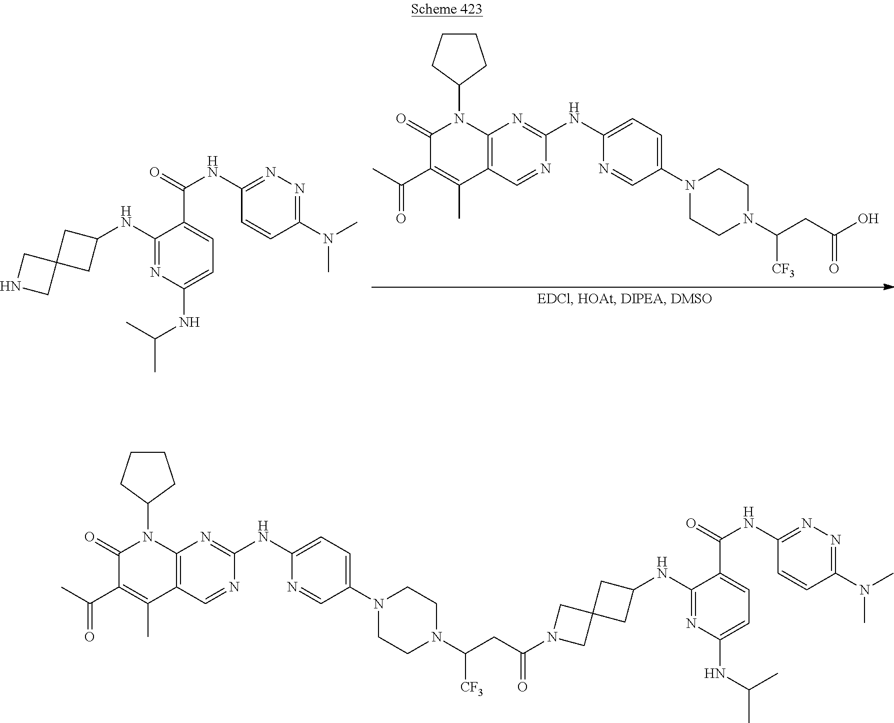

CPD-367 was synthesized following the standard procedure for preparing CPD-143 (3.7 mg, 44% yield) as a yellow solid. MS (ESI) m/z=980.7 [M+H]⁺.

### Example 424. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-2-azaspiro[3.3]heptan-6-yl)amino)-N-(6-(dimethylamino)pyridazin-3-yl)-6-(isopropylamino)nicotinamide (CPD-368)

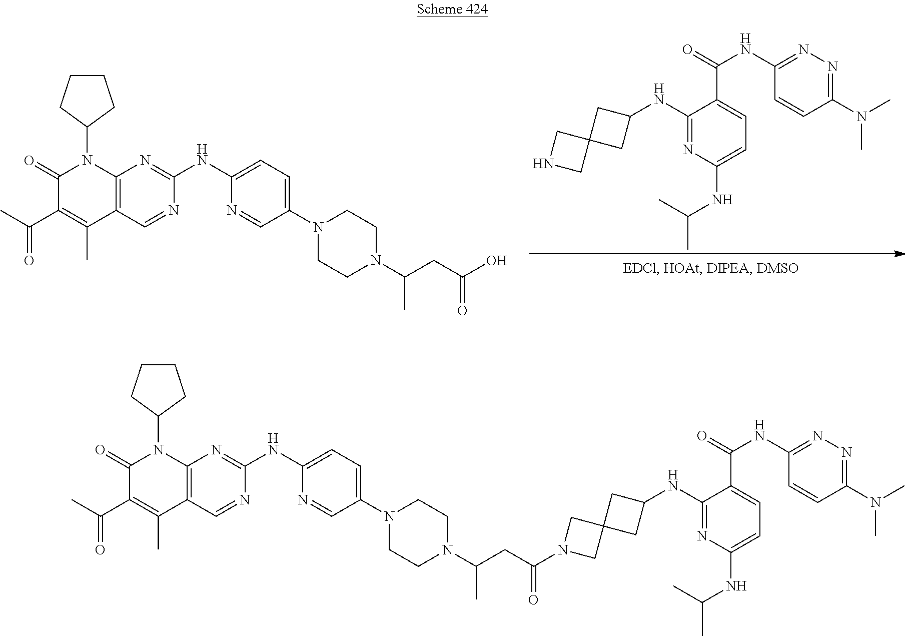 [SMILES: CC(=O)c1c(C)c2cnc(Nc3ccc(N4CCN(C(C)CC(=O)O)CC4)cn3)nc2n(C2CCCC2)c1=O ; CC(C)Nc1ccc(C(=O)Nc2ccc(N(C)C)nn2)c(NC2CC3(CNC3)C2)n1 ; CC(=O)c1c(C)c2cnc(Nc3ccc(N4CCN(C(C)CC(=O)N5CC6(CC(Nc7nc(NC(C)C)ccc7C(=O)Nc7ccc(N(C)C)nn7)C6)C5)CC4)cn3)nc2n(C2CCCC2)c1=O]

CPD-368 was synthesized following the standard procedure for preparing CPD-143 (3.5 mg, 45% yield) as a yellow solid. MS (ESI) m/z=926.6 [M+H]⁺.

### Example 425. 2-((1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetyl)piperidin-4-yl)amino)-N-(6-(dimethylamino)pyridazin-3-yl)-6-(isopropylamino)nicotinamide (CPD-369)

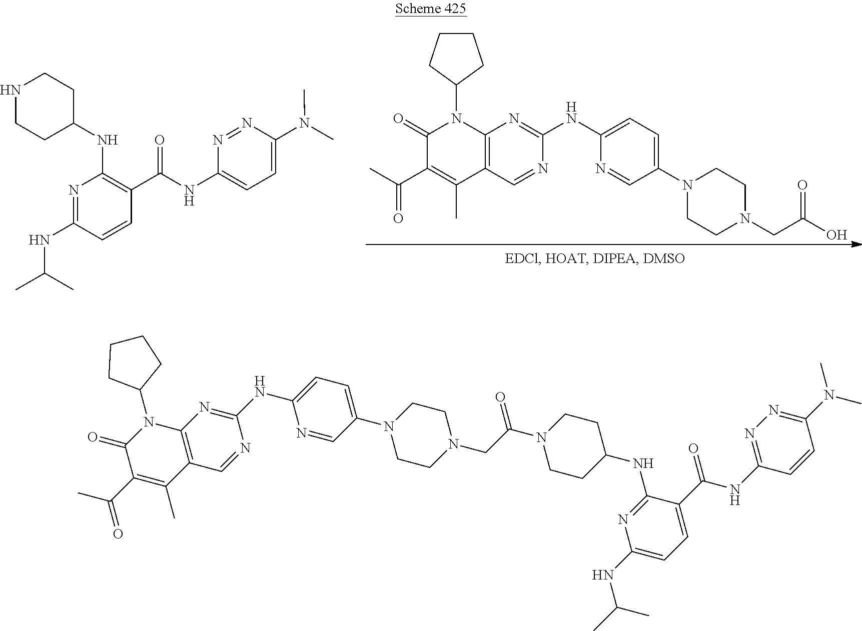

CPD-369 was synthesized following the standard procedure for preparing CPD-143 (1.7 mg, 10% yield) as a yellow solid. MS (ESI) m/z=886.6 [M+H]⁺.

### Example 426. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)pyrrolidin-3-yl)amino)-N-(6-(dimethylamino)pyridazin-3-yl)-6-(isopropylamino)nicotinamide (CPD-370)

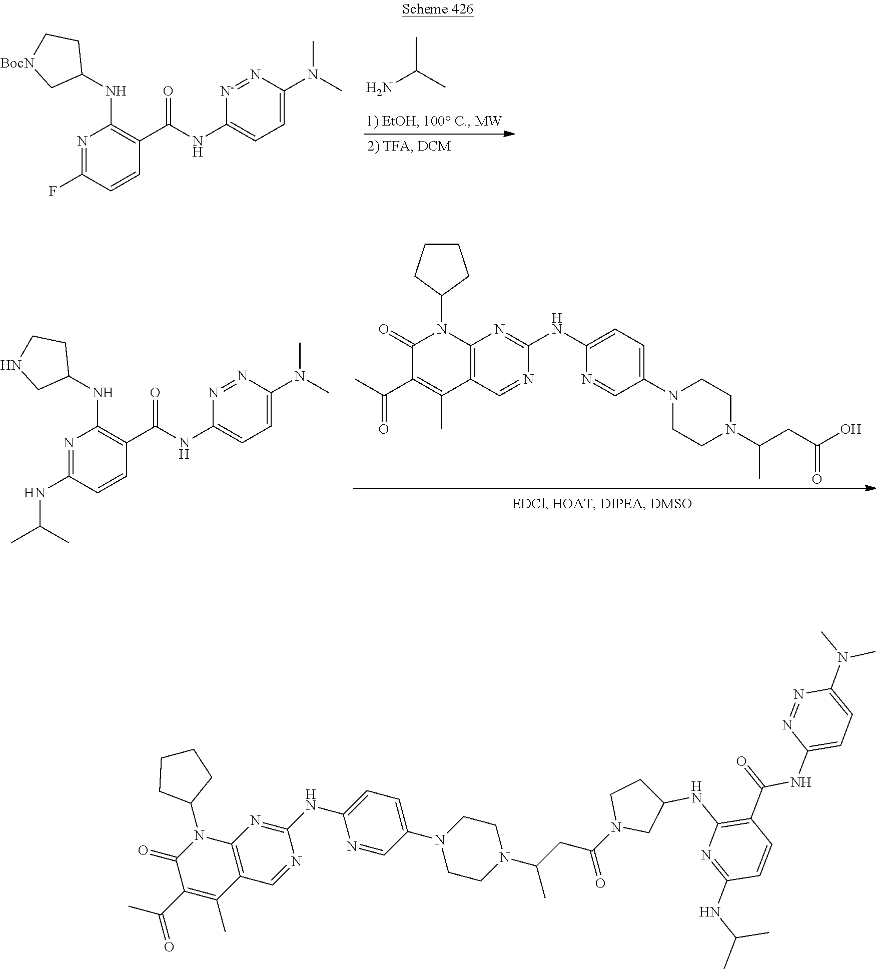

CPD-370 was synthesized following the standard procedures for preparing CPD-144 (1.6 mg, 6% yield over 3 steps) as a yellow solid. MS (ESI) m/z=900.8 [M+H]⁺.

### Example 427. 2-((6-(1-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)cyclopropane-1-carboxamido)spiro[3.3]heptan-2-yl)amino)-6-(isopropylamino)-N-(5-methylisoxazol-3-yl)nicotinamide (CPD-371)

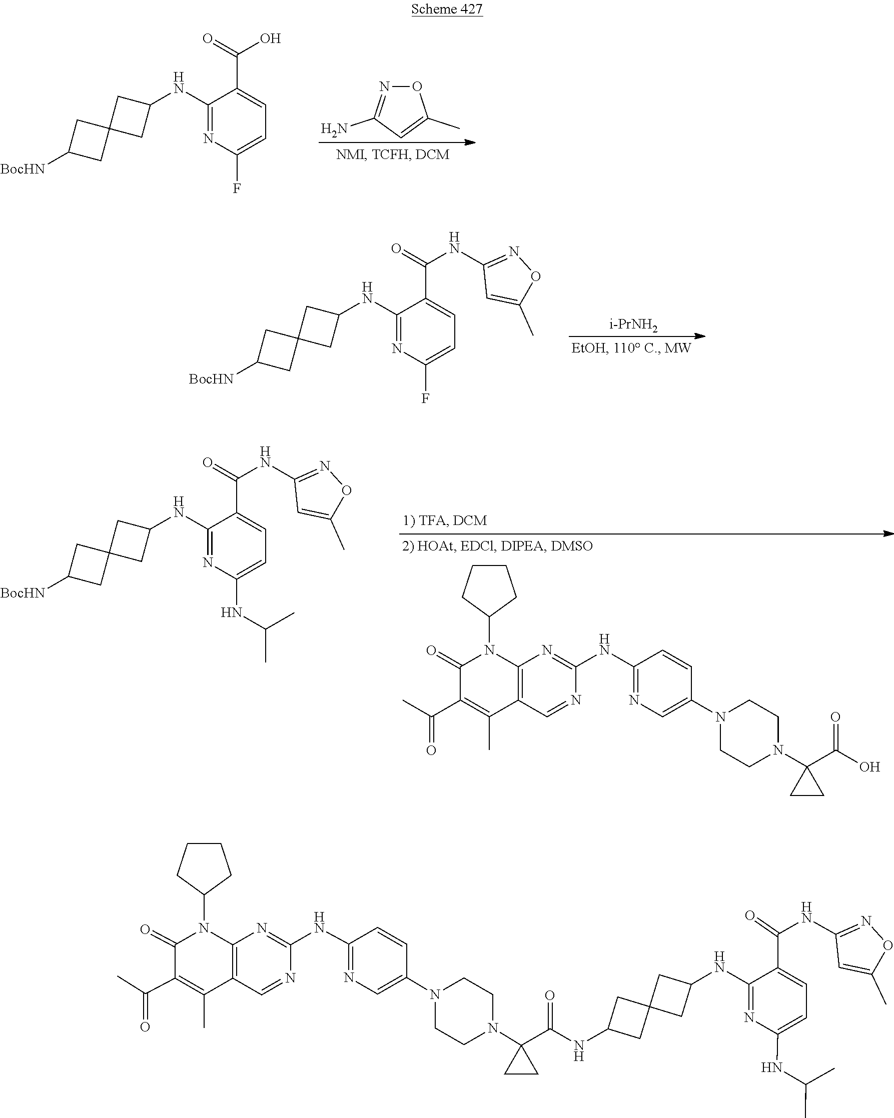

CPD-371 was synthesized following the standard procedures for preparing CPD-144 (2.1 mg, 2% yield over 4 steps) as a yellow solid. MS (ESI) m/z=898.7 [M+H]⁺.

### Example 428. 2-((1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)glycyl)pyrrolidin-3-yl)amino)-N-(6-(dimethylamino)pyridazin-3-yl)-6-(isopropylamino)nicotinamide (CPD-372)

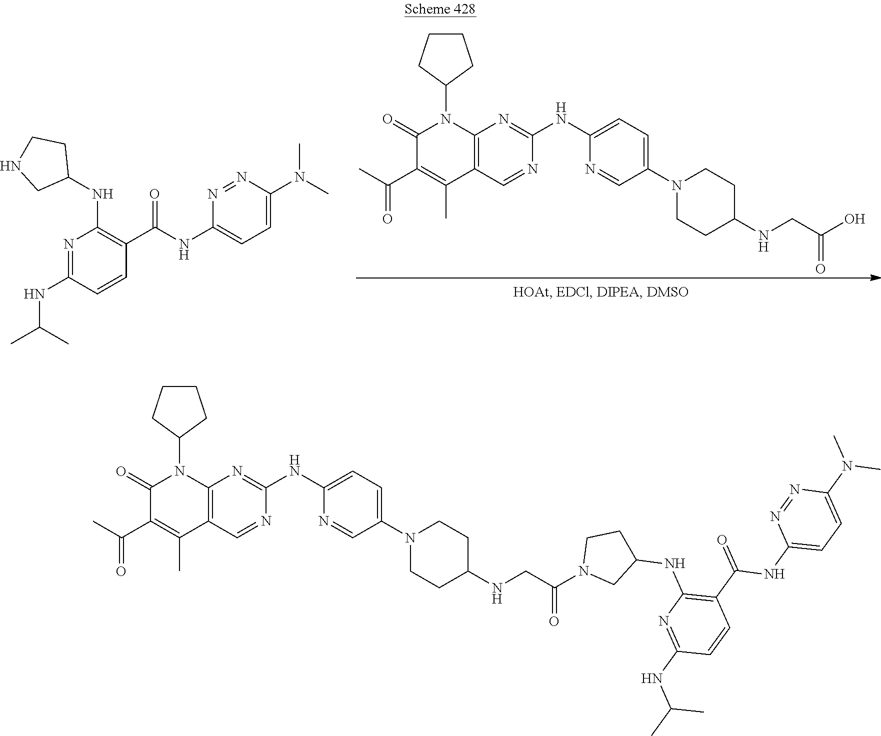

CPD-372 was synthesized following the standard procedure for preparing CPD-143 (1.9 mg, 11% yield) as a yellow solid. MS (ESI) m/z=886.7 [M+H]⁺.

### Example 429. 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octan-2-1)acetyl)-2-azaspiro[3.3]heptan-6-yl)amino)-N-(6-(dimethylamino)pyridazin-3-yl)-6-((methyl-d3)amino)nicotinamide (CPD-373)

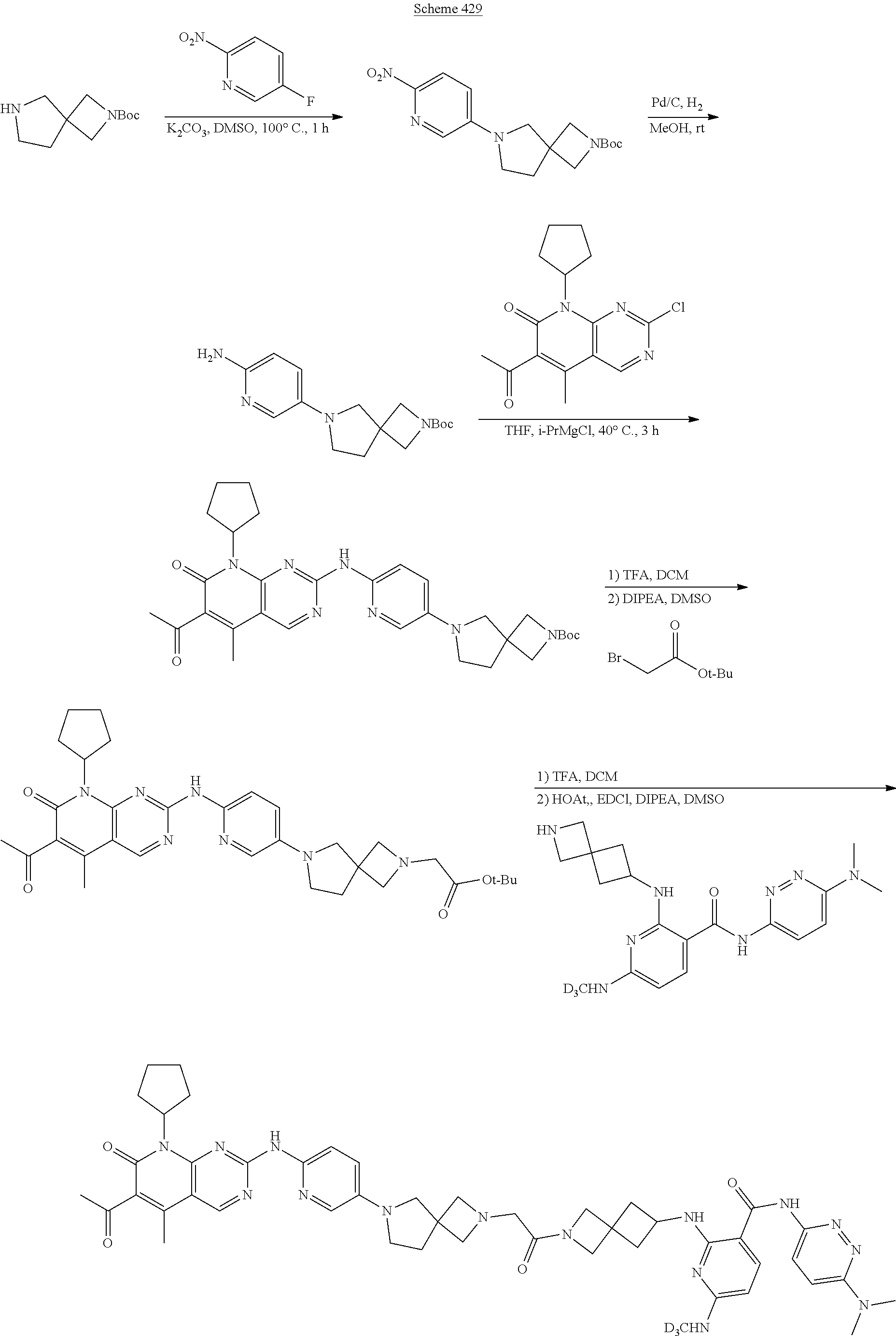

### Step 1. Synthesis of tert-butyl 6-(6-nitropyridin-3-yl)-2,6-diazaspiro[3.4]octane-2-carboxylate

To a solution of tert-butyl 2, 7-diazaspiro[3.4]octane-2-carboxylate (500 mg, 2.36 mmol) in DMSO (2 mL) were added K₂CO₃ (488 mg, 3.53 mmol) and 5-fluoro-2-nitro-pyridine (334 mg, 2.36 mmol). The mixture was stirred at 100° C. for 1 h. The mixture was poured into H₂O (30 mL) and filtered. The filter cake was dissolved in acetonitrile (20 mL) and concentrated under vacuum to provide the desired product (800 mg, 99% yield) as a yellow solid. ¹HNMR (400 MHz, CDCl₃) δ 8.17-8.11 (m, 1H), 7.18 (dd, J=3.2, 9.2 Hz, 1H), 4.70-4.66 (m, 1H), 4.19-4.11 (m, 1H), 3.45-3.35 (m, 4H), 2.37 (t, J=10.0 Hz, 2H), 1.79-1.66 (m, 6H), 1.45 (s, 9H). MS (ESI) m/z 335.3 [M+H]⁺.

### Step 2. Synthesis of tert-butyl 6-(6-aminopyridin-3-yl)-2,6-diazaspiro[3.4]octane-2-carboxylate

To a solution of tert-butyl 7-(6-nitro-3-pyridyl)-2,7-diazaspiro[3.4]octane-2-carboxylate (800 mg, 2.39 mmol) in MeOH (30 mL) was added 10% Pd/C (200 mg) at rt. The mixture was stirred at rt for 1 h under H₂ (15 psi). The resulting mixture was filtered and concentrated under vacuum to provide the desired product (720 mg, 94% yield) as a red gum. ¹HNMR (400 MHz, DMSO-d₆) δ 7.33 (d, J=2.8 Hz, 1H), 6.83 (dd, J=2.8, 8.8 Hz, 1H), 6.39 (d, J=8.8 Hz, 1H), 3.82-3.78 (m, 4H), 3.18-3.12 (m, 4H), 2.12 (t, J=6.8 Hz, 2H), 1.38 (s, 9H). MS (ESI) m/z=305.3 [M+H]⁺.

### Step 3. Synthesis of tert-butyl 6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octane-2-carboxylate

To a solution of tert-butyl 6-(6-aminopyridin-3-yl)-2,6-diazaspiro[3.4]octane-2-carboxylate (398 mg, 1.31 mmol) in THF (4 mL) was added chloro(isopropyl)magnesium (2 M, 1.96 mL) at rt. The mixture was stirred at rt for 1 h. Then 6-acetyl-2-chloro-8-cyclopentyl-5-methyl-pyrido[2,3-d]pyrimidin-7-one (200 mg, 654 μmol) in THF (2 mL) was added into the mixture at rt. The mixture was stirred at 40° C. for 2 h. The reaction was quenched with saturated NH₄Cl solution (10 mL) and extracted with ethyl acetate (10 mL×3). The combined organic layers were washed with brine (10 mL), dried over Na₂SO₄, filtered and concentrated. The residue was purified by column chromatography (SiO₂, petroleum ether/ethyl acetate=10:1 to 3:1) to provide the desired product (250 mg, 63% yield) as a yellow solid. ¹HNMR (400 MHz, CDCl₃) δ 8.80 (s, 1H), 8.11 (d, J=9.2 Hz, 1H), 7.71 (d, J=2.8 Hz, 1H), 6.96 (dd, J=2.8, 9.2 Hz, 1H), 5.88 (q, J=8.8 Hz, 1H), 4.00-3.88 (m, 4H), 3.50 (s, 2H), 3.41 (t, J=6.8 Hz, 2H), 2.56 (s, 3H), 2.42-2.31 (m, 5H), 2.28-2.21 (m, 2H), 2.11-2.02 (m, 2H), 1.93-1.84 (m, 2H), 1.71-1.67 (d, J=5.2 Hz, 2H), 1.47 (s, 9H). MS (ESI) m/z=574.3 [M+H]⁺.

### Step 4. Synthesis of 2-((5-(2,6-diazaspiro[3.4]octan-6-yl)pyridin-2-yl)amino)-6-acetyl-8-cyclopentyl-5-methylpyrido[2,3-d]pyrimidin-7(8H)-one

To a solution of tert-butyl 6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octane-2-carboxylate (10 mg, 0.017 mmol) in dichloromethane (1 mL) was added TFA (0.2 mL). The reaction mixture was stirred at rt for 1 h. The mixture was concentrated to give the crude product (8 mg, 97% yield) as a yellow oil. The residue was used directly in the next step without further purification. MS (ESI) m/z=474.5 [M+H]⁺.

### Step 5. Synthesis of tert-butyl 2-(6-(6-((6-acetyl-8-cyclopentyl-5-methyl-7-oxo-7,8 dihydro-pyrido[2,3-d]pyrimidin-2-yl)amino)pyridin-3-yl)-2,6-diazaspiro[3.4]octan-2-yl)acetate

To a solution of 2-((5-(2,6-diazaspiro[3.4]octan-6-yl)pyridin-2-yl)amino)-6-acetyl-8-cyclopentyl-5-methylpyrido[2,3-d]pyrimidin-7(8H)-one (8 mg, 0.017 mmol) and tert-butyl 2-bromoacetate (4 mg, 0.02 mmol) in DMSO (1 mL) was added DIPEA (0.1 mL). The reaction mixture was stirred at rt for 12 h. The mixture was purified by reverse-phase chromatography to give the desired product (9 mg, 910% yield) as a yellow oil. MS (ESI) m/z=588.5 [M+H]⁺.

### Step 6. Synthesis of 2-(6-(6-((6-acetyl-8-cyclopentyl-5-methyl-7-oxo-7,8-dihydropyrido[2,3d]pyrimidin-2-yl)amino)pyridin-3-yl)-2,6-diazaspiro[3.4]octan-2-yl)acetic acid

To a solution of tert-butyl 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octan-2-yl)acetate (9 mg, 0.015 mmol) in dichloromethane (1 mL) was added TFA (0.2 mL). The reaction mixture was stirred at rt for 1 h. The mixture was concentrated to give the crude product (8 mg, 98% yield) as a yellow oil. The residue was used directly in the next step without further purification. MS (ESI) m/z=532.5 [M+H]⁺.

### Step 7. Synthesis of 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octan-2-yl)acetyl)-2-azaspiro[3.3]heptan-6-yl)amino)-N-(6-(dimethylamino)pyridazin-3-yl)-6-((methyl-d₃)amino)nicotinamide

To a solution of 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octan-2-yl)acetic acid (8 mg, 0.015 mmol), 2-((2-azaspiro[3.3]heptan-6-yl)amino)-N-(6-(dimethylamino)pyridazin-3-yl)-6-((methyl-d₃)amino)nicotinamide (6 mg, 0.015 mmol), EDCI (6 mg, 0.03 mmol) and HOAT (4 mg, 0.03 mmol) in DMSO (1 mL) was added DIPEA (0.1 mL). The reaction mixture was stirred at rt for 12 h. The mixture was purified by prep-HPLC to give the desired product (1.9 mg, 14% yield) as a yellow solid. MS (ESI) m/z=899.7 [M+H]⁺.

### Example 430. 2-((6-((7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonan-2-yl)carbamoyl)spiro[3.3]heptan-2-yl)amino)-N-(6-(dimethylamino)pyridazin-3-yl)-6-(methylamino)nicotinamide (CPD-374)

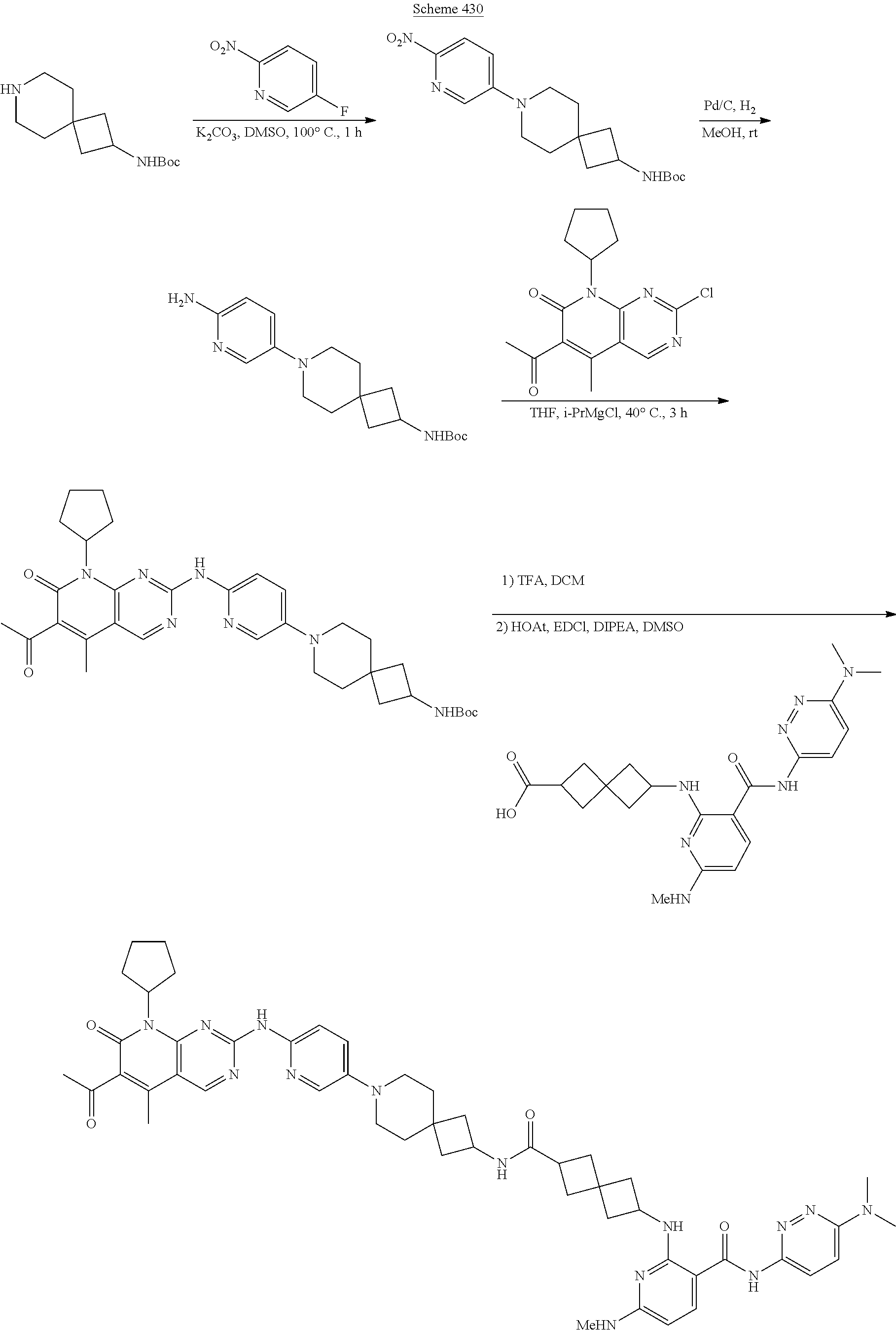

CPD-374 was synthesized following the standard procedures for preparing CPD-373 (2.1 mg, 2% yield over 5 steps) as a yellow solid. MS (ESI) m/z=909.6 [M+H]⁺.

### Example 431. 2-((3-((7-(6-((6-acetyl-8-cyclopentyl-5-methyl-7-oxo-7,8-dihydropyrido[2,3-d]pyrimidin-2-yl)amino)pyridin-3-yl)-7-azaspiro[3.5]nonan-2-yl)carbamoyl)cyclobutyl)amino)-N-(6-(dimethylamino)pyridazin-3-yl)-6-(methylamino)nicotinamide (CPD-375)

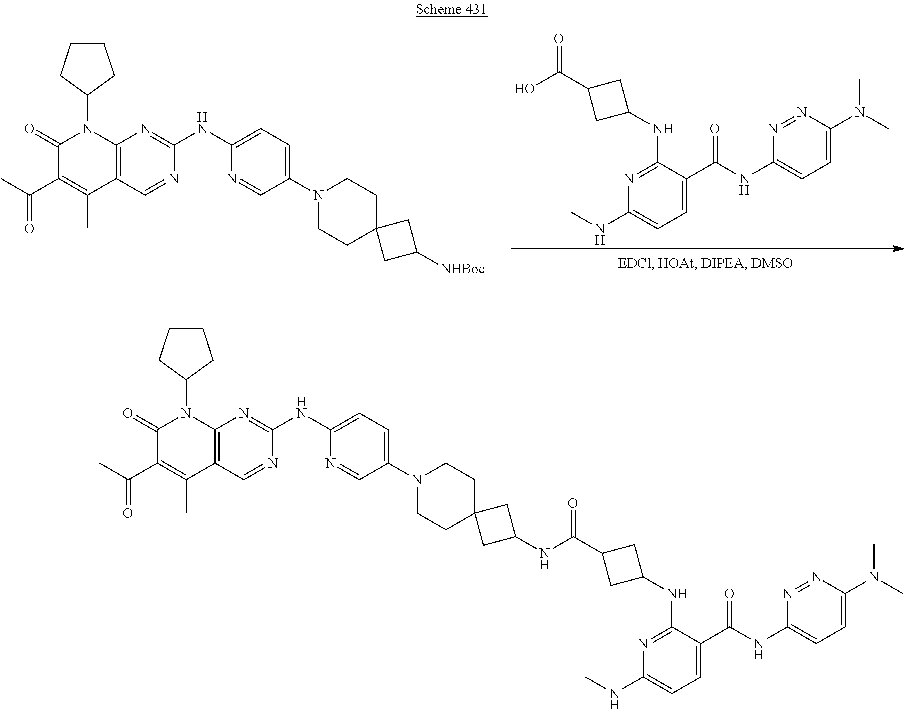 [SMILES: CC(=O)c1c(C)c2cnc(Nc3ccc(N4CCC5(CC4)CC(NC(=O)OC(C)(C)C)C5)cn3)nc2n(C2CCCC2)c1=O ; CNc1ccc(C(=O)Nc2ccc(N(C)C)nn2)c(NC2CC(C(=O)O)C2)n1 ; CNc1ccc(C(=O)Nc2ccc(N(C)C)nn2)c(NC2CC(C(=O)NC3CC4(CCN(c5ccc(Nc6ncc7c(C)c(C(C)=O)c(=O)n(C8CCCC8)c7n6)nc5)CC4)C3)C2)n1]

CPD-375 was synthesized following the standard procedure for preparing CPD-143 (2.6 mg, 30% yield) as a yellow solid. MS (ESI) m/z=869.7 [M+H]⁺.

### Example 432. 2-((6-(3-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-1-carbonyl)spiro[3.3]heptan-2-yl)amino)-N-(6-(dimethylamino)pyridazin-3-yl)-6-(methylamino)nicotinamide (CPD-376)

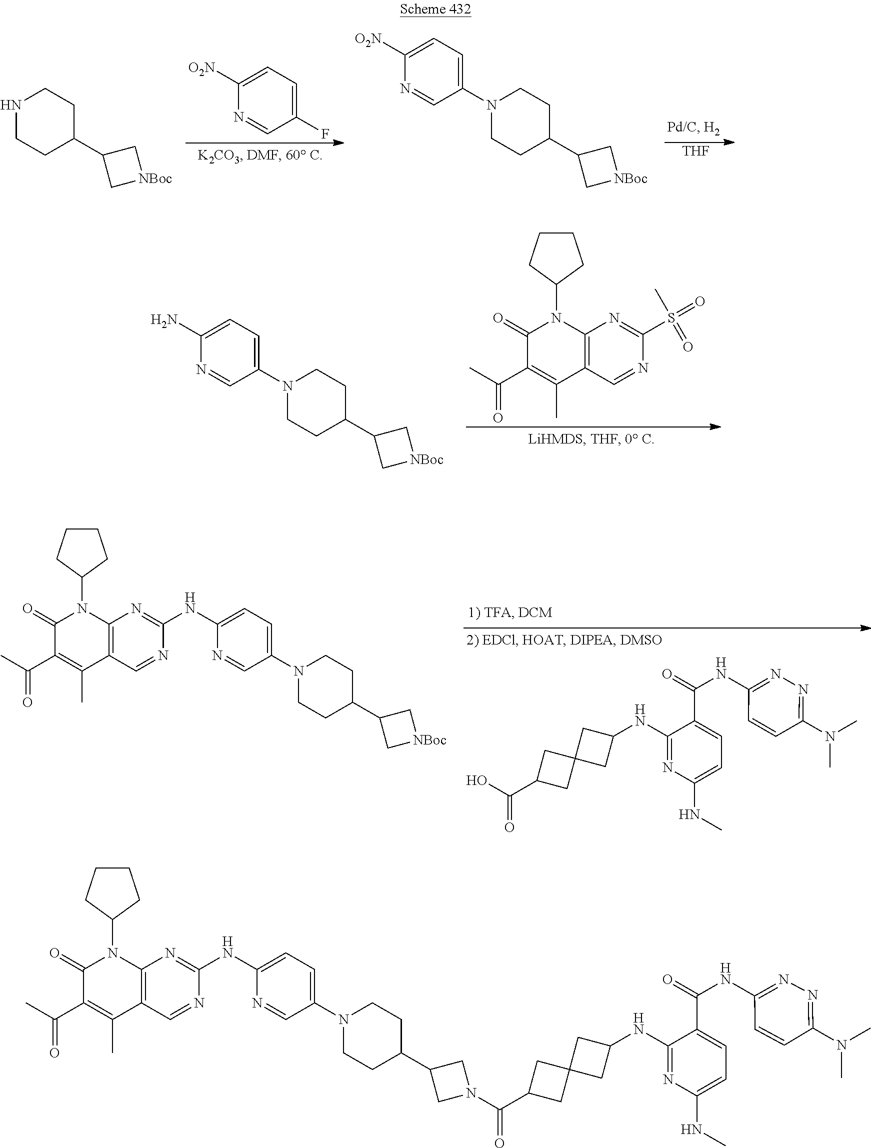

CPD-376 was synthesized following the standard procedures for preparing CPD-364 (2.1 mg, 3% yield over 5 steps) as a yellow solid. MS (ESI) m/z=909.7 [M+H]⁺.

### Example 433. 2-((3-(1-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethyl)cyclobutyl)amino)-6-(isopropylamino)-N-(5-methylisoxazol-3-yl)nicotinamide (CPD-377)

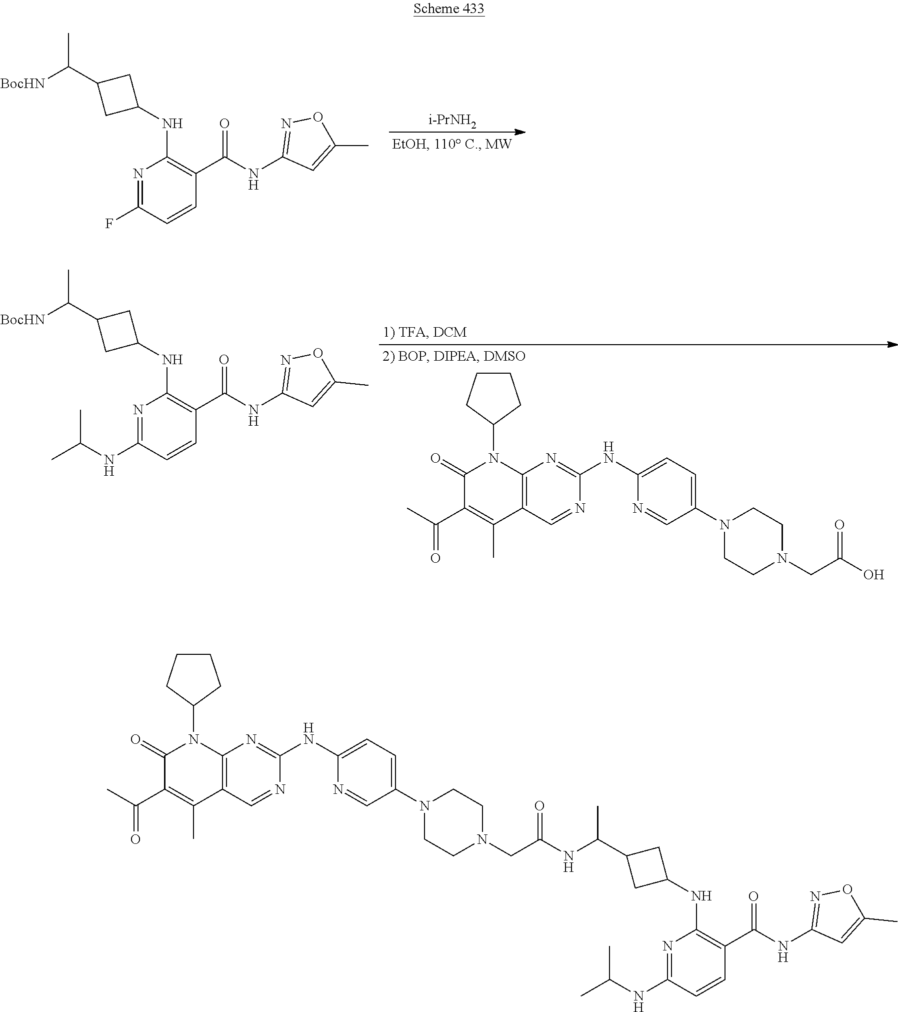 [SMILES: "i-PrNH2" ; Cc1cc(NC(=O)c2ccc(F)nc2NC2CC(C(C)NC(=O)OC(C)(C)C)C2)no1 ; Cc1cc(NC(=O)c2ccc(NC(C)C)nc2NC2CC(C(C)NC(=O)OC(C)(C)C)C2)no1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)NC(C)C5CC(Nc6nc(NC(C)C)ccc6C(=O)Nc6cc(C)on6)C5)CC4)cn3)nc2n(C2CCCC2)c1=O]

CPD-376 was synthesized following the standard procedures for preparing CPD-144 (5.0 mg, 17% yield over 3 steps) as a yellow solid. MS (ESI) m/z=860.7 [M+H]⁺.

### Example 434. 2-((6-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.4]octane-2-carbonyl)spiro[3.3]heptan-2-yl)amino)-N-(6-(dimethylamino)pyridazin-3-yl)-6-((methyl-d₃)amino)nicotinamide (CPD-378)

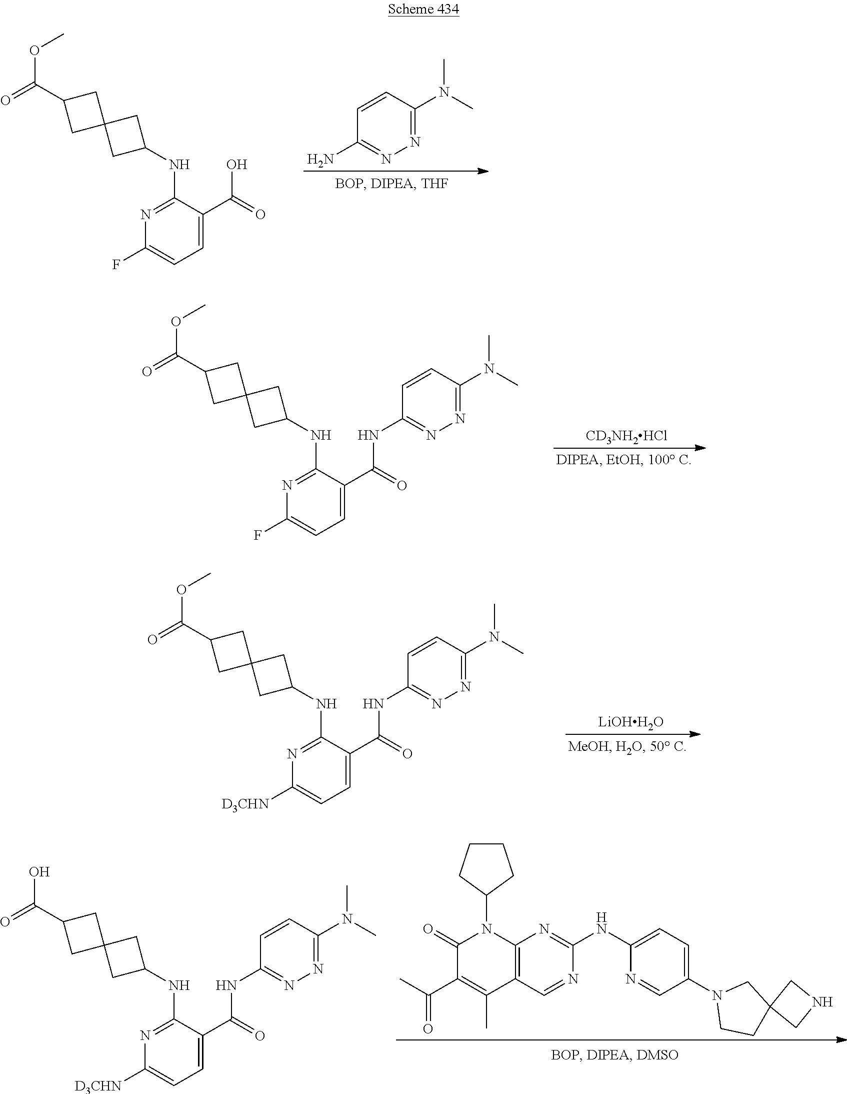

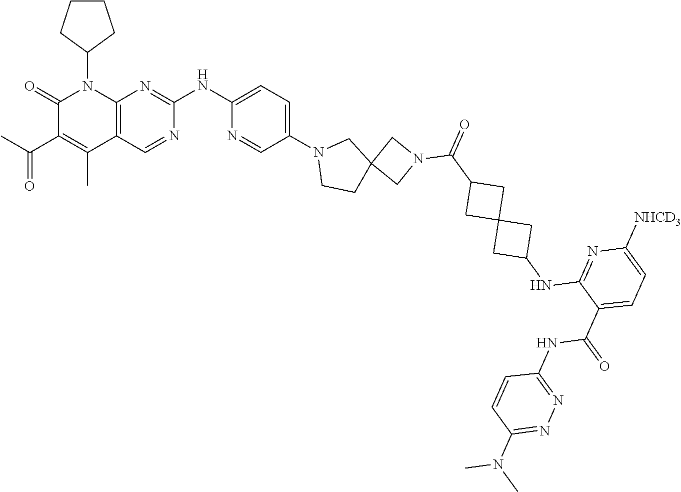

CPD-378 was synthesized following the standard procedures for preparing CPD-82 (7.0 mg, 12% yield over 4 steps) as a yellow solid. MS (ESI) m/z=884.8 [M+H]⁺.

### Example 435. 2-((6-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonane-2-carbonyl)spiro[3.3]heptan-2-yl)amino)-N-(6-(dimethylamino)pyridazin-3-yl)-6-((methyl-d₃)amino)nicotinamide (CPD-379)

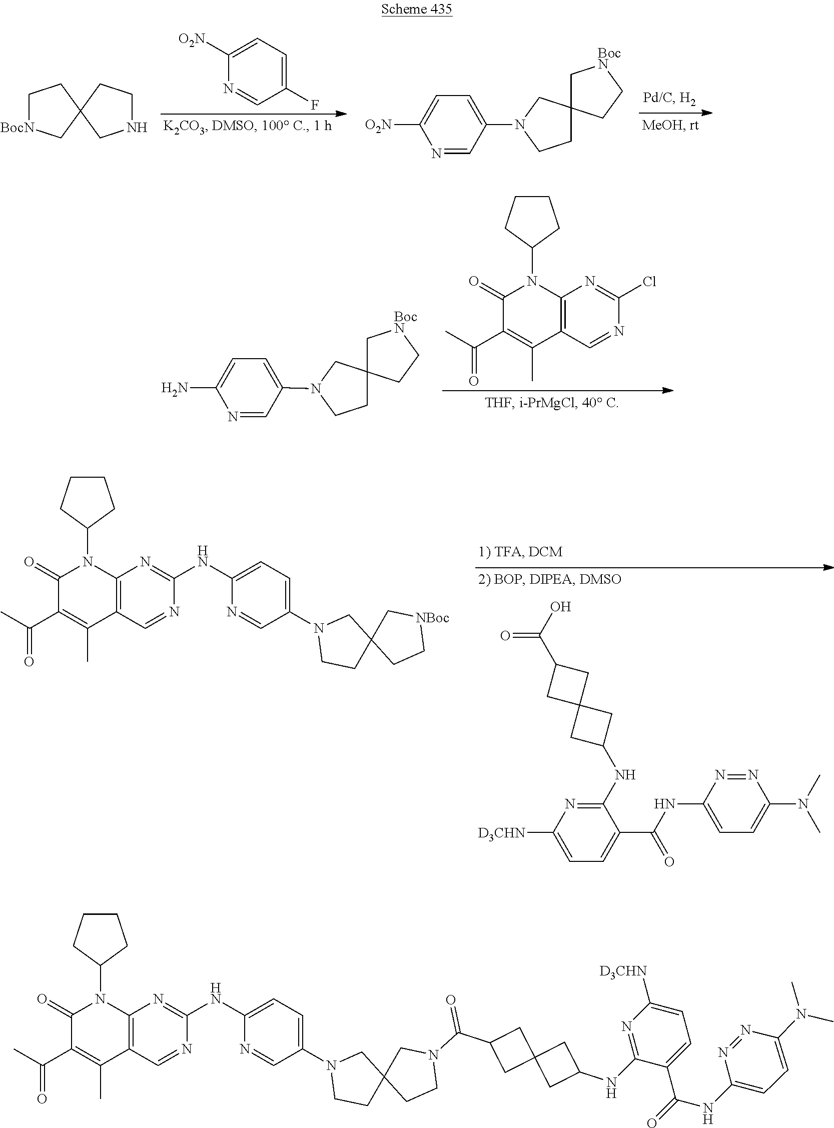

CPD-379 was synthesized following the standard procedures for preparing CPD-373 (8.0 mg, 2%0 yield over 5 steps) as a yellow solid. MS (ESI) m/z=898.7 [M+H]⁺.

### Example 436. 2-((6-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)spiro[3.3]heptan-2-yl)amnion)-N-(6-(dimethylamino)pyridazin-3-yl)-6-((methyl-d3)amnion)nicotinamide (CPD-380)

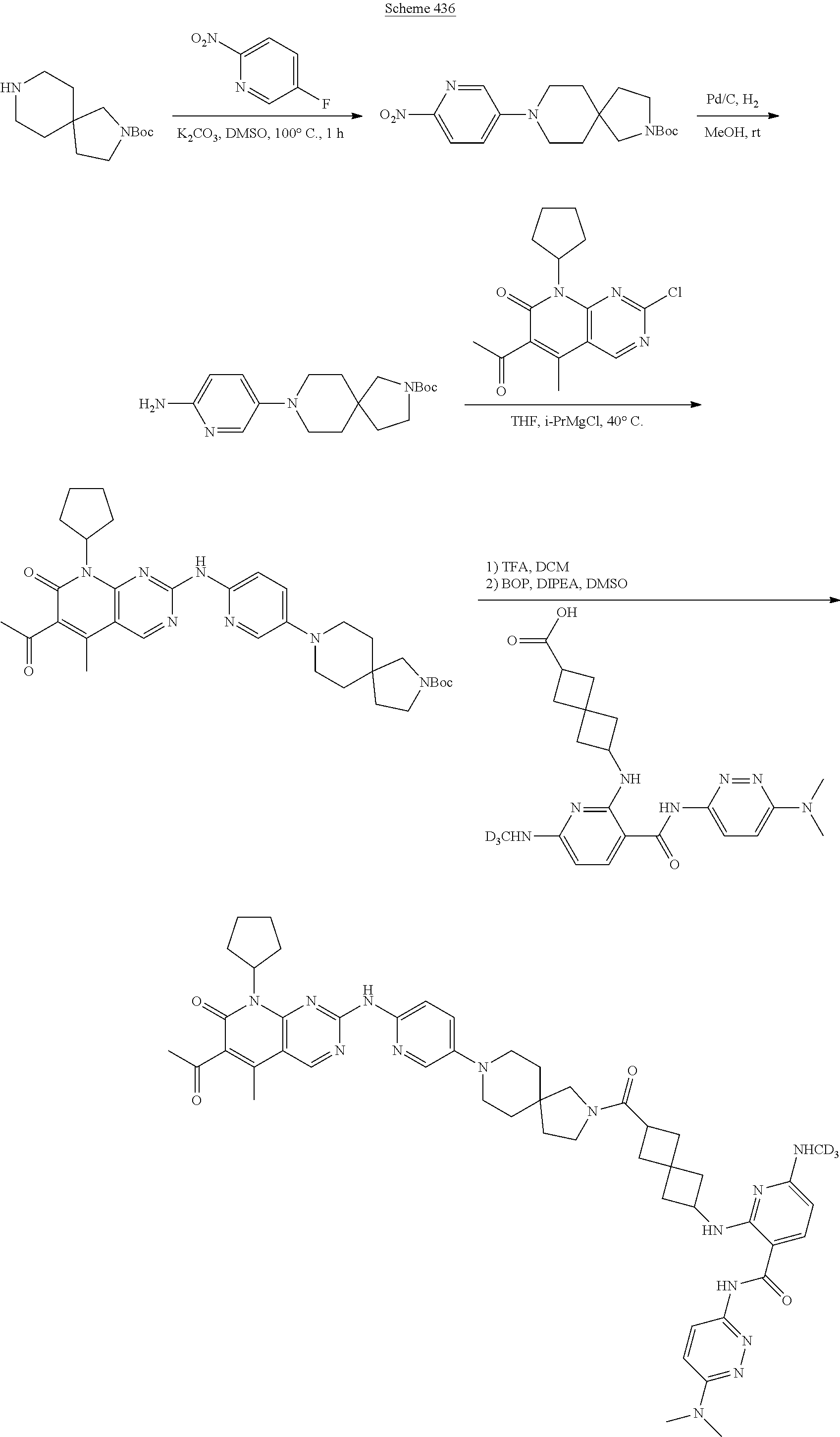

CPD-380 was synthesized following the standard procedures for preparing CPD-373 (9.0 mg, 6% yield over 5 steps) as a yellow solid. MS (ESI) m/z=912.8 [M+H]⁺.

### Example 437. 2-((3-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)cyclobutyl)amino)-N-(6-(dimethylamino)pyridazin-3-yl)-6-(methylamino)nicotinamide (CPD-381)

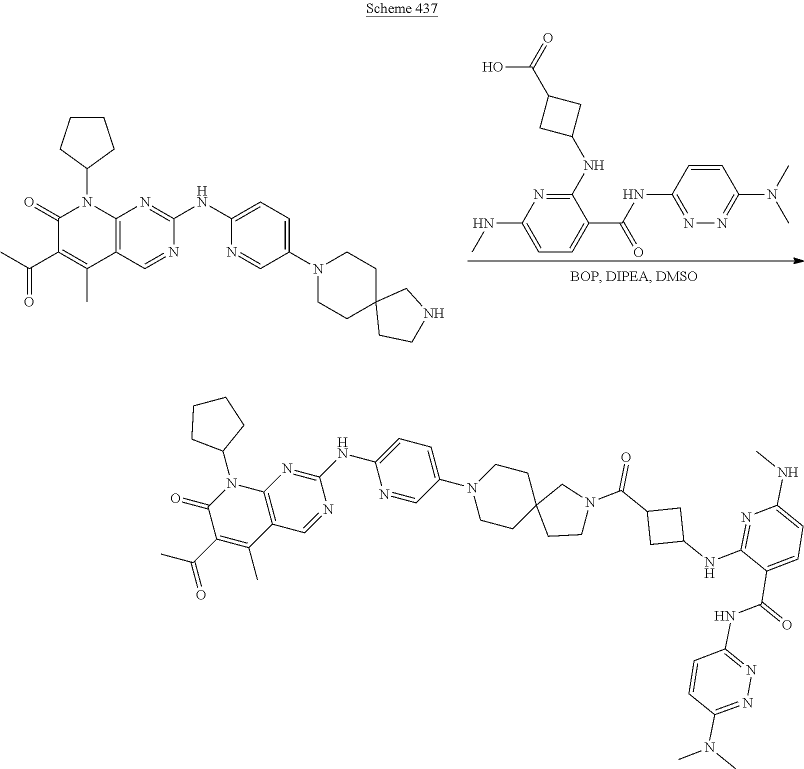

CPD-381 was synthesized following the standard procedure for preparing CPD-261 (7.0 mg, 33% yield) as a yellow solid. MS (ESI) m/z=869.6 [M+H]⁺.

### Example 438. 2-((3-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)amino)acetamido)cyclobutyl)amino)-N-(6-(dimethylamino)pyridazin-3-yl)-6-(isopropylamino)nicotinamide (CPD-382)

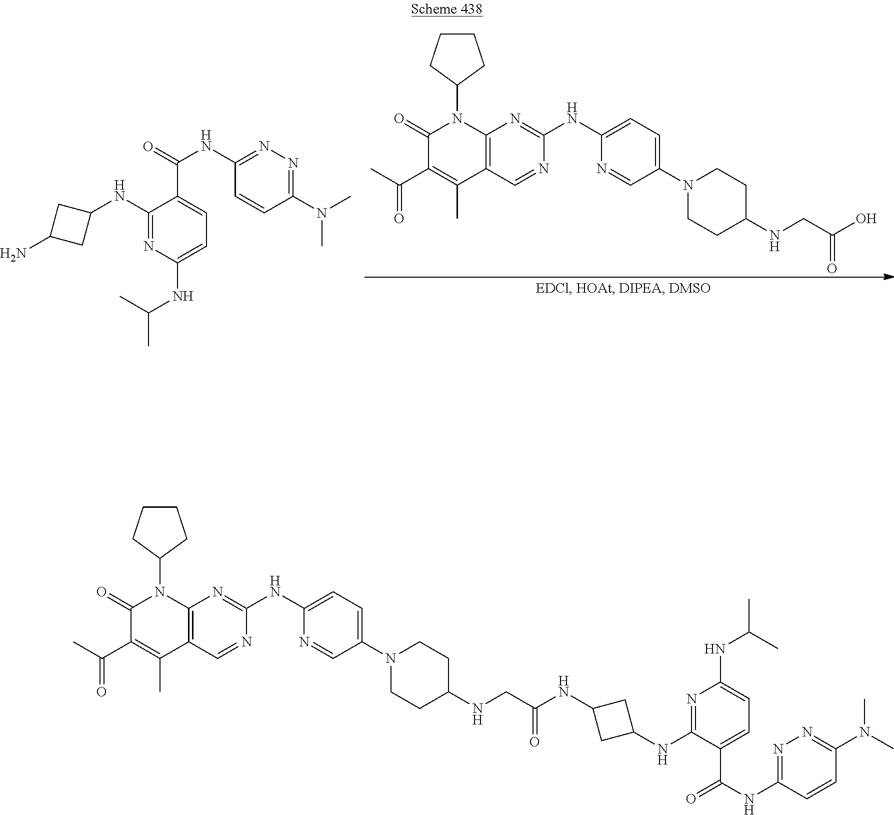

CPD-382 was synthesized following the standard procedure for preparing CPD-143 (2.1 mg, 9% yield) as a yellow solid. MS (ESI) m/z=886.6 [M+H]⁺.

### Example 439. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)-6-azaspiro[2.5]octan-1-yl)amino)-N-(6-(dimethylamino)pyridazin-3-yl)-6-((methyl-d₃)amino)nicotinamide (CPD-383)

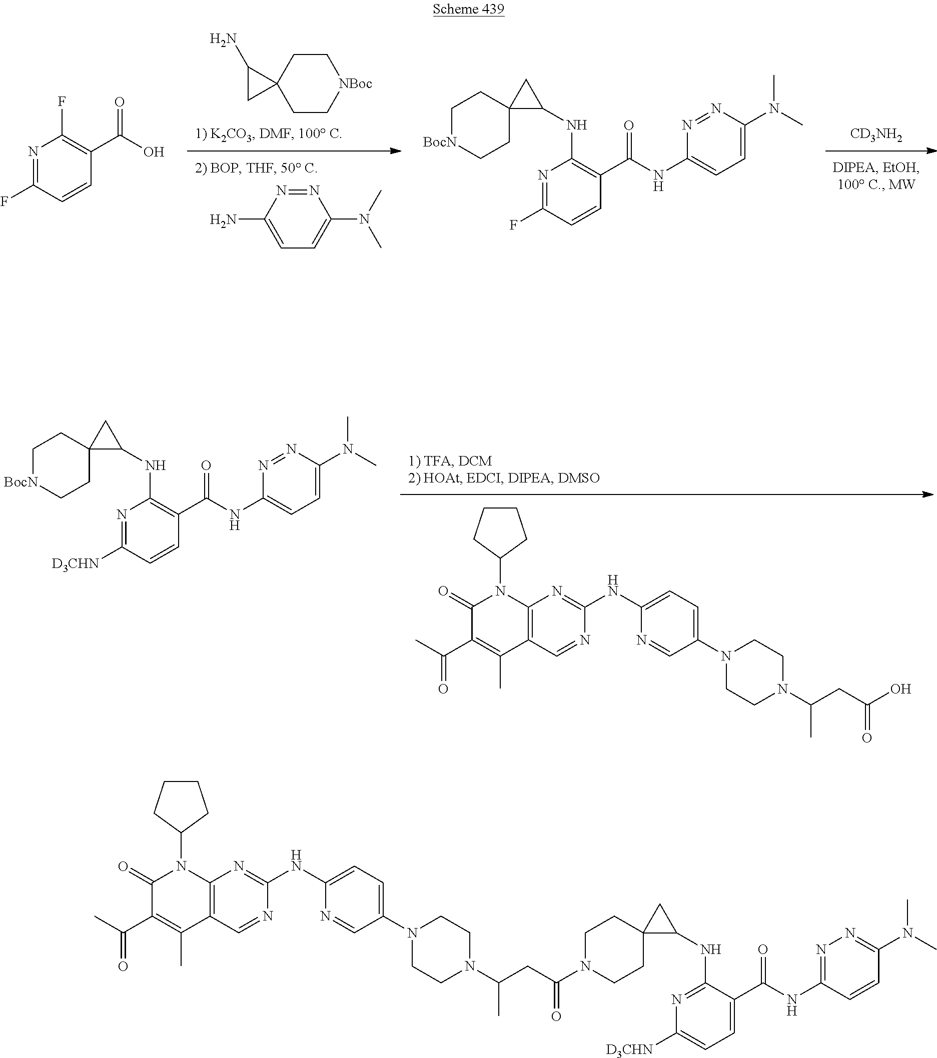

CPD-383 was synthesized following the standard procedures for preparing CPD-145 (1.1 mg, 2% yield over 5 steps) as a yellow solid. MS (ESI) m/z=915.8 [M+H]⁺.

### Example 440. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6-(dimethylamino)pyridazin-3-yl)-6-((methyl-d₃)amino)nicotinamide (CPD-384)

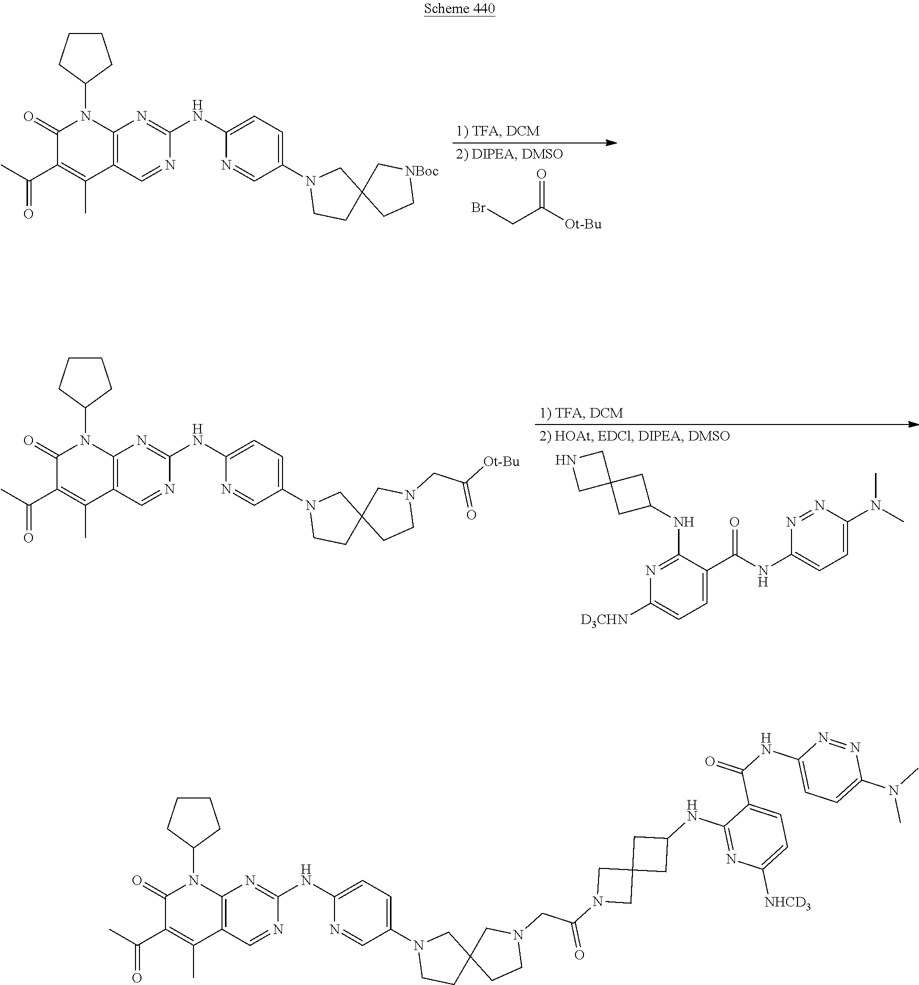

CPD-384 was synthesized following the standard procedures for preparing CPD-373 (2.8 mg, 18% yield over 4 steps) as a yellow solid. MS (ESI) m/z=913.8 [M+H]⁺.

### Example 441. 2-((4-(8-(6-((6-acetyl-8-cyclopentyl-5-methyl-7-oxo-7,8-dihydropyrido[2,3-d]pyrimidin-2-yl)amino)pyridin-3-yl)-2,8-diazaspiro[4.5]decane-2-carbonyl)cyclohexyl)amino)-N-(6-(dimethylamino)pyridazin-3-yl)-6-(methylamino)nicotinamide (CPD-385)

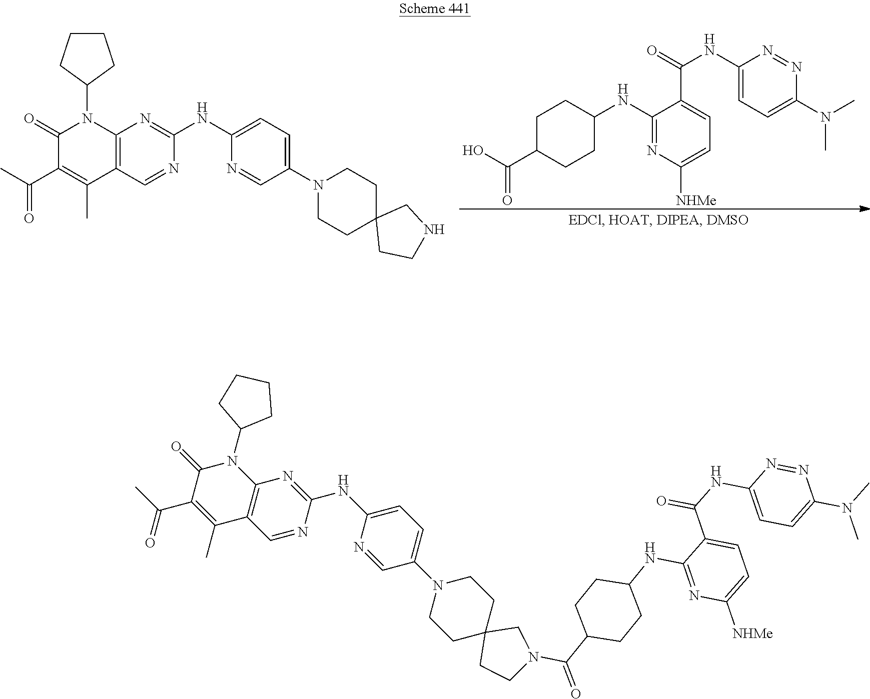 [SMILES: CC(=O)c1c(C)c2cnc(Nc3ccc(N4CCC5(CCNC5)CC4)cn3)nc2n(C2CCCC2)c1=O ; CNc1ccc(C(=O)Nc2ccc(N(C)C)nn2)c(NC2CCC(C(=O)O)CC2)n1 ; CNc1ccc(C(=O)Nc2ccc(N(C)C)nn2)c(NC2CCC(C(=O)N3CCC4(CCN(c5ccc(Nc6ncc7c(C)c(C(C)=O)c(=O)n(C8CCCC8)c7n6)nc5)CC4)C3)CC2)n1]

CPD-385 was synthesized following the standard procedure for preparing CPD-143 (5.0 mg, 33% yield) as a yellow solid. MS (ESI) m/z=897.8 [M+H]⁺.

### Example 442. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperidin-1-yl)acetyl)piperidin-4-yl)amino)-N-(6-(dimethylamino)pyridazin-3-yl)-6-(isopropylamino)nicotinamide (CPD-386)

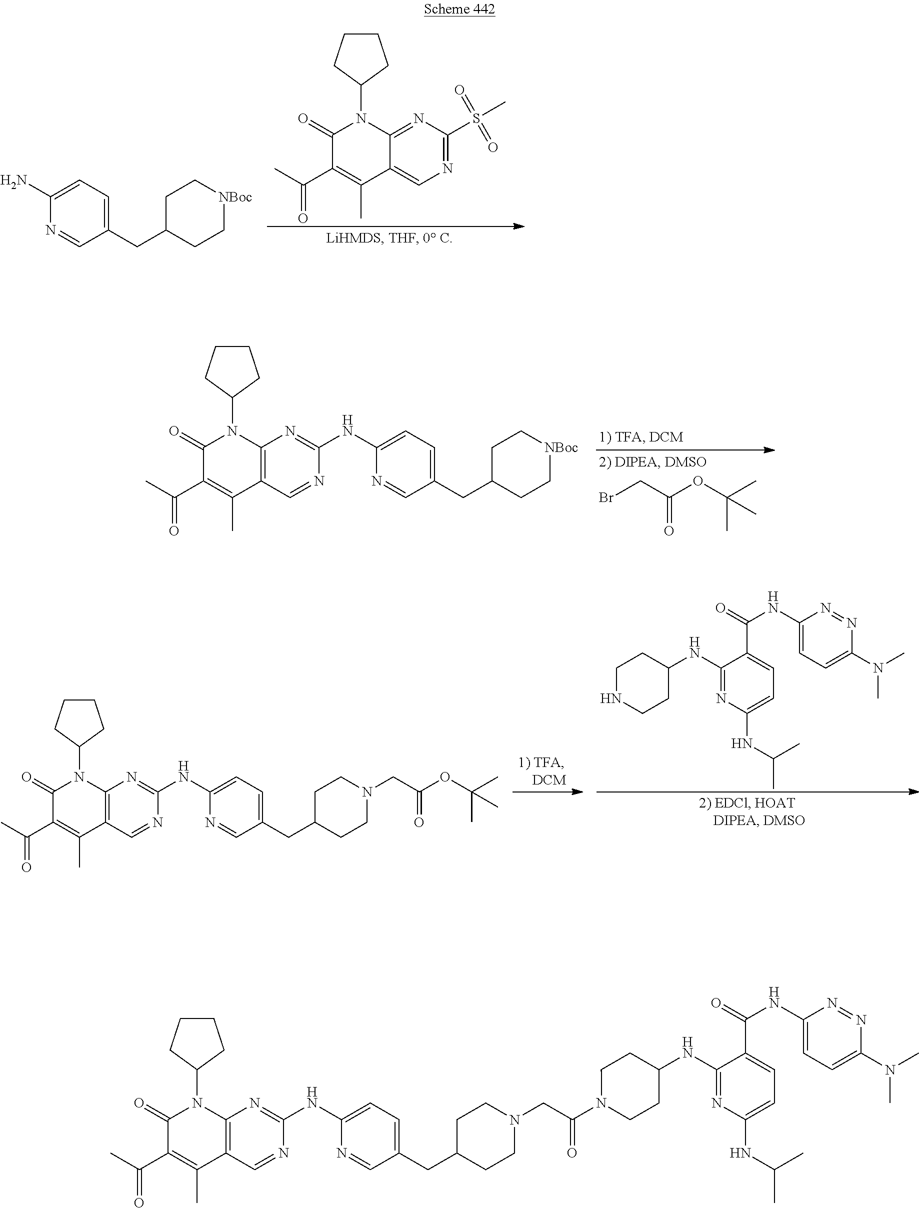

CPD-386 was synthesized following the standard procedures for preparing CPD-364 and CPD-373 (14.0 mg, 13% yield over 5 steps) as a yellow solid. MS (ESI) m/z=899.7 [M+H]⁺.

### Example 443. 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propan-2-yl)cyclobutyl)amino)-N-(1,5-dimethyl-1H-pyrazol-3-yl)-6-(isopropylamino)nicotinamide (CPD-387)

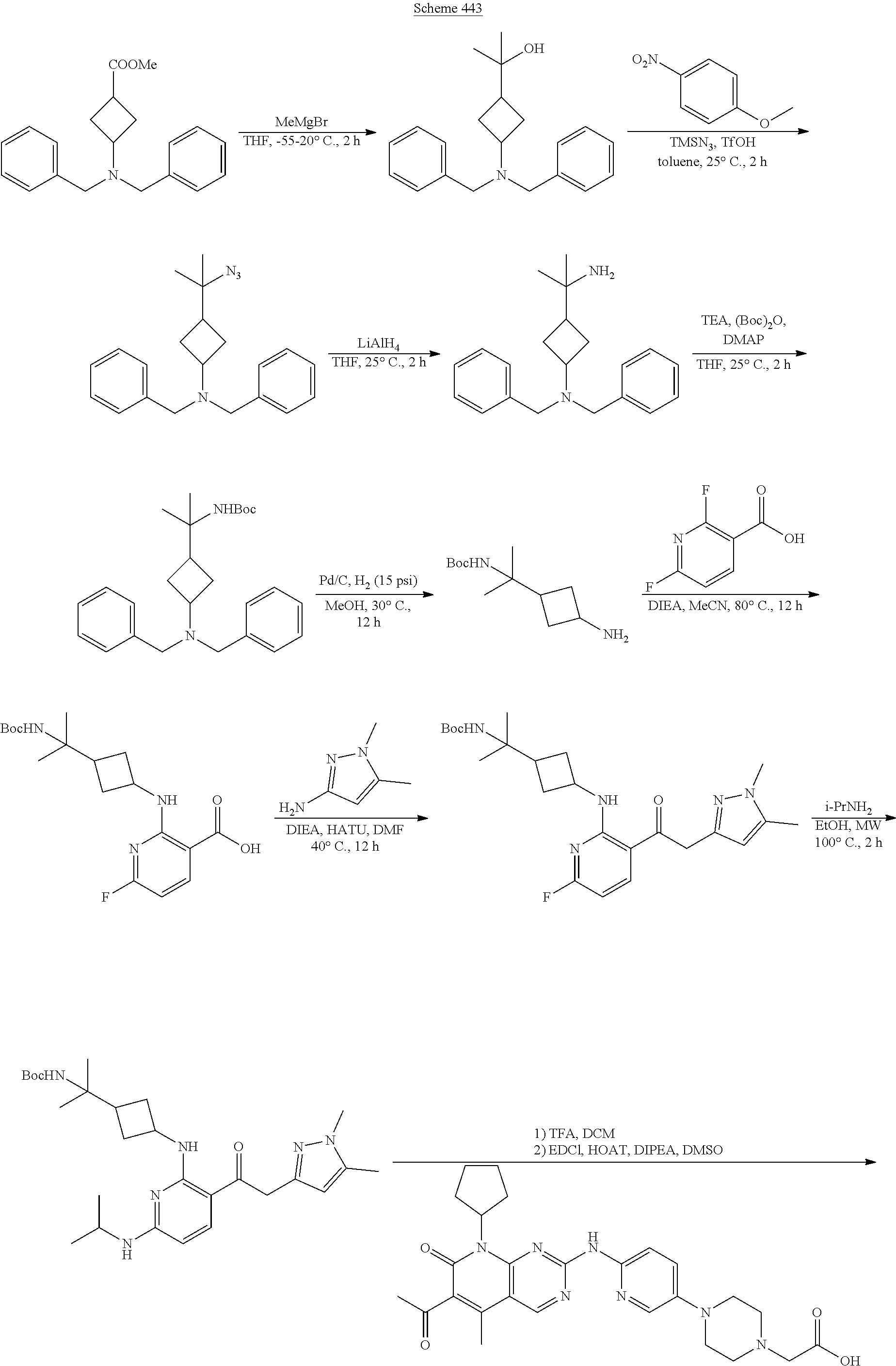

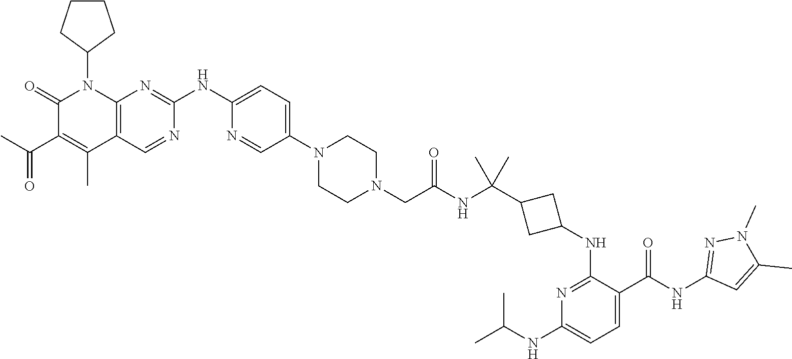 [SMILES: CC(=O)c1c(C)c2cnc(Nc3ccc(N4CCN(CC(=O)NC(C)(C)C5CC(Nc6nc(NC(C)C)ccc6C(=O)Nc6cc(C)n(C)n6)C5)CC4)cn3)nc2n(C2CCCC2)c1=O]

### Step 1. Synthesis of 2-(3-(dibenzylamino)cyclobutyl)propan-2-ol

To a solution of methyl 3-(dibenzylamino)cyclobutanecarboxylate (5.00 g, 16.1 mmol) in THF (50 mL) was added MeMgBr (3 M, 32 mL) at −55 [symbol] . The mixture was stirred at 20 [symbol] for 2 h. The reaction mixture was quenched with saturated NH₄Cl solution (50 mL) and extracted with ethyl acetate (20 mL×3). The combined organic layers were washed with brine (50 mL), dried over Na₂SO₄, filtered and concentrated. The residue was purified by column chromatography on silica (petroleum ether/ethyl acetate=50:1 to 10:1) to provide the desired product (5.00 g, 99% yield) as a yellow oil. MS (ESI) m/z=310.1 [M+H]⁺.

### Step 2. Synthesis of 3-(2-azidopropan-2-yl)-N,N-dibenzylcyclobutan-1-amine

To a solution of 2-[3-(dibenzylamino)cyclobutyl]propan-2-ol (2.00 g, 6.46 mmol), 1-methoxy-4-nitro-benzene (494 mg, 3.23 mmol) and TMSN₃ (2.23 g, 19.3 mmol) in toluene (20 mL) was added trifluoromethanesulfonic acid (1.94 g, 12.9 mmol) at 25° C. The mixture was stirred at 25 [symbol] for 2 h. The reaction mixture was concentrated and purified by column chromatography on silica (petroleum ether/ethyl acetate=10:1 to 1:1) to provide the desired product (1.60 g, 74% yield) as a yellow oil. MS (ESI) m/z=335.4 [M+H]⁺.

### Step 3. Synthesis of 3-(2-aminopropan-2-yl)-N,N-dibenzylcyclobutan-1-amine

To a solution of 3-(1-azido-1-methyl-ethyl)-N,N-dibenzyl-cyclobutanamine (1.60 g, 4.78 mmol) in THF (16 mL) was added LiAlH₄ (2.5 M, 7.65 mL). The mixture was stirred at 25 [symbol] for 2 h. The reaction mixture was quenched with Na₂SO₄·10H₂O (8.00 g) and filtered through a Celite pad. The filtrate was evaporated in vacuo to provide the desired product as a yellow oil (1. 10 g, 74% yield). MS (ESI) m/z=309.1 [M+H]⁺.

### Step 4. Synthesis of tert-butyl (2-(3-(dibenzylamino)cyclobutyl)propan-2-yl)carbamate

To a solution of 3-(1-amino-1-methyl-ethyl)-N,N-dibenzyl-cyclobutanamine (1.35 g, 4.38 mmol) and DMAP (54 mg, 442 μmol) in THF (13 mL) were added (Boc)₂O (1.28 g, 5.88 mmol) and TEA (981 mg, 9.69 mmol). The mixture was stirred at 25° C. for 2 h. The reaction mixture was concentrated. The residue was purified by column chromatography on silica gel (petroleum ether/ethyl acetate=50:1 to 10:1) to provide the desired product (1.10 g, 62% yield) as a white solid. MS (ESI) m/z=409.2 [M+H]⁺.

### Step 5. Synthesis of tert-butyl (2-(3-aminocyclobutyl)propan-2-yl)carbamate

A mixture of tert-butyl N-[1-[3-(dibenzylamino)cyclobutyl]-1-methyl-ethyl]carbamate (1.10 g, 2.69 mmol) and 10% Pd/C (100 mg) in MeOH (20 mL) was degassed and purged with N₂ for 3 times. The mixture was stirred at 30 [symbol] for 12 h under H₂ atmosphere (15 psi). The reaction mixture was filtered through a celite pad and the filtrate was evaporated in vacuo to provide the desired product (600 mg, 97% yield) as a yellow oil. ¹HNMR (400 MHz, CDCl₃) δ 3.24-3.18 (m, 1H), 2.24-2.21 (m, 1H), 1.65 (s, 4H), 1.44 (s, 9H), 1.22 (s, 6H).

The remaining steps were performed according to the procedures for preparing CPD-353 to provide the desired product (2.3 mg, 1% yield for 5 steps) as a yellow solid. MS (ESI) m/z=887.8 [M+H]⁺.

### Example 444. 2-((3-((3-(6-((6-acetyl-8-cyclopentyl-5-methyl-7-oxo-7,8-dihydropyrido[2,3-d]pyrimidin-2-yl)amino)pyridin-3-yl)-3-azaspiro[5.5]undecan-9-yl)carbamoyl)cyclobutyl)amino)-N-(6-(dimethylamino)pyridazin-3-yl)-6-(methylamino)nicotinamide (CPD-388)

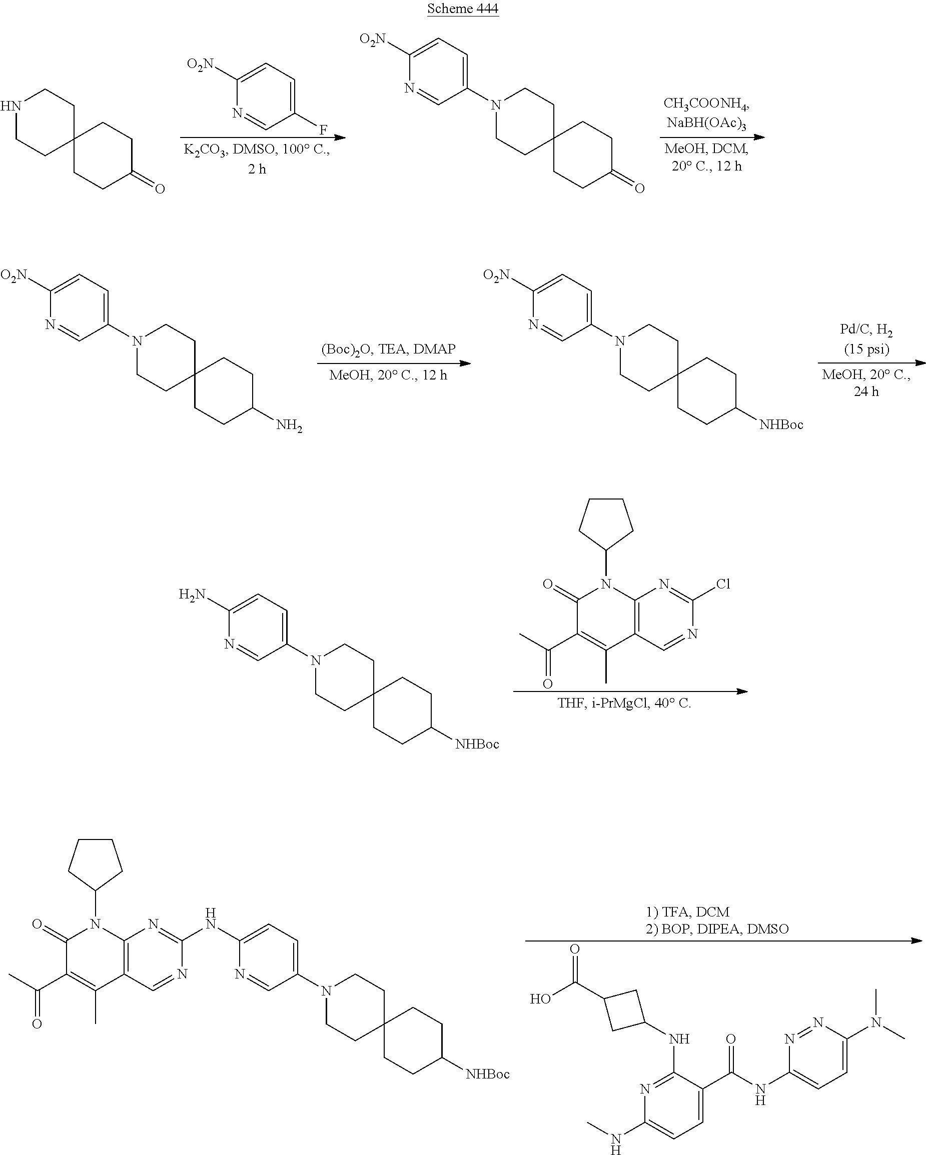

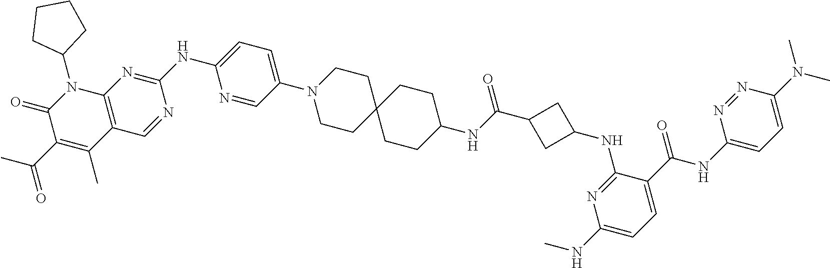 [SMILES: CNc1ccc(C(=O)Nc2ccc(N(C)C)nn2)c(NC2CC(C(=O)NC3CCC4(CC3)CCN(c3ccc(Nc5ncc6c(C)c(C(C)=O)c(=O)n(C7CCCC7)c6n5)nc3)CC4)C2)n1]

### Step 1. Synthesis of 3-(6-nitro-3-pyridyl)-3-azaspiro[5.5]undecan-9-one

To a mixture of 3-azaspiro[5.5]undecan-9-one (1.20 g, 5.89 mmol) and 5-fluoro-2-nitro-pyridine (800 mg, 5.63 mmol) in DMSO (10 mL) was added K₂CO₃ (1.60 g, 11.6 mmol, 2.20 mL) at rt. The reaction mixture was stirred at 100 [symbol] for 2 h. After cooling to rt, the mixture was poured into H₂O (50 mL) and extracted with dichloromethane (50 mL×3). The combined organic phase was washed with brine (100 mL×6), dried with anhydrous Na₂SO₄, filtered, and concentrated in vacuum to provide the desired product (2.27 g, crude) as a yellow solid. MS (ESI) m/z=290.2 [M+H]⁺.

### Step 2. Synthesis of 3-(6-nitro-3-pyridyl)-3-azaspiro[5.5]undecan-9-amine

To a mixture of 3-(6-nitro-3-pyridyl)-3-azaspiro[5.5]undecan-9-one (1.10 g, 3.80 mmol) in MeOH (40 mL) and dichloromethane (15 mL) was added CH₃COONH₄ (6.60 g, 85.6 mmol) at 20 [symbol] . After stirring at rt for 10 min, NaBH(OAc)₃ (2.20 g, 10.4 mmol) was added into the above mixture at rt. The reaction mixture was stirred at rt for additional 12 h. The mixture was poured into H₂O (100 mL) and extracted with dichloromethane (100 mL×3). The combined organic phase was dried with anhydrous Na₂SO₄, filtered, and concentrated in vacuum to provide the desired product (1. 10 g, 99% yield) as a yellow solid. MS (ESI) m/z=291.3 [M+H]⁺.

### Step 3. Synthesis of tert-butyl N-[3-(6-nitro-3-pyridyl)-3-azaspiro[5.5]undecan-9-yl]carbamate

To a mixture of 3-(6-nitro-3-pyridyl)-3-azaspiro[5.5]undecan-9-amine (1.10 g, 3.79 mmol) in dichloromethane (10 mL) were added TEA (1.45 g, 14.34 mmol, 2 mL), (Boc)₂O (1.90 g, 8.71 mmol, 2 mL) and DMAP (50 mg, 409 mol) at 20° C. The reaction mixture was stirred at 20° C. for 12 h. The mixture was poured into H₂O (50 mL) and extracted with dichloromethane (100 mL×5). The combined organic phase was dried with anhydrous Na₂SO₄, filtered, and concentrated in vacuum to provide the desired product (1.00 g, 67% yield) as a yellow oil. MS (ESI) m/z=391.3 [M+H]⁺.

The remaining steps were performed according to the procedures for preparing CPD-373 to provide the desired product (7 mg, 5% yield over 4 steps) as a yellow solid. MS (ESI) m/z=897.8 [M+H]⁺.

### Example 445. 2-((1-((4-(6-((6-acetyl-8-cyclopentyl-5-methyl-7-oxo-7,8-dihydropyrido[2,3-d]pyrimidin-2-yl)amino)pyridin-3-yl)cyclohexyl)glycyl)piperidin-4-yl)amino)-N-(6-(dimethylamino)pyridazin-3-yl)-6-(isopropylamino)nicotinamide (CPD-389)

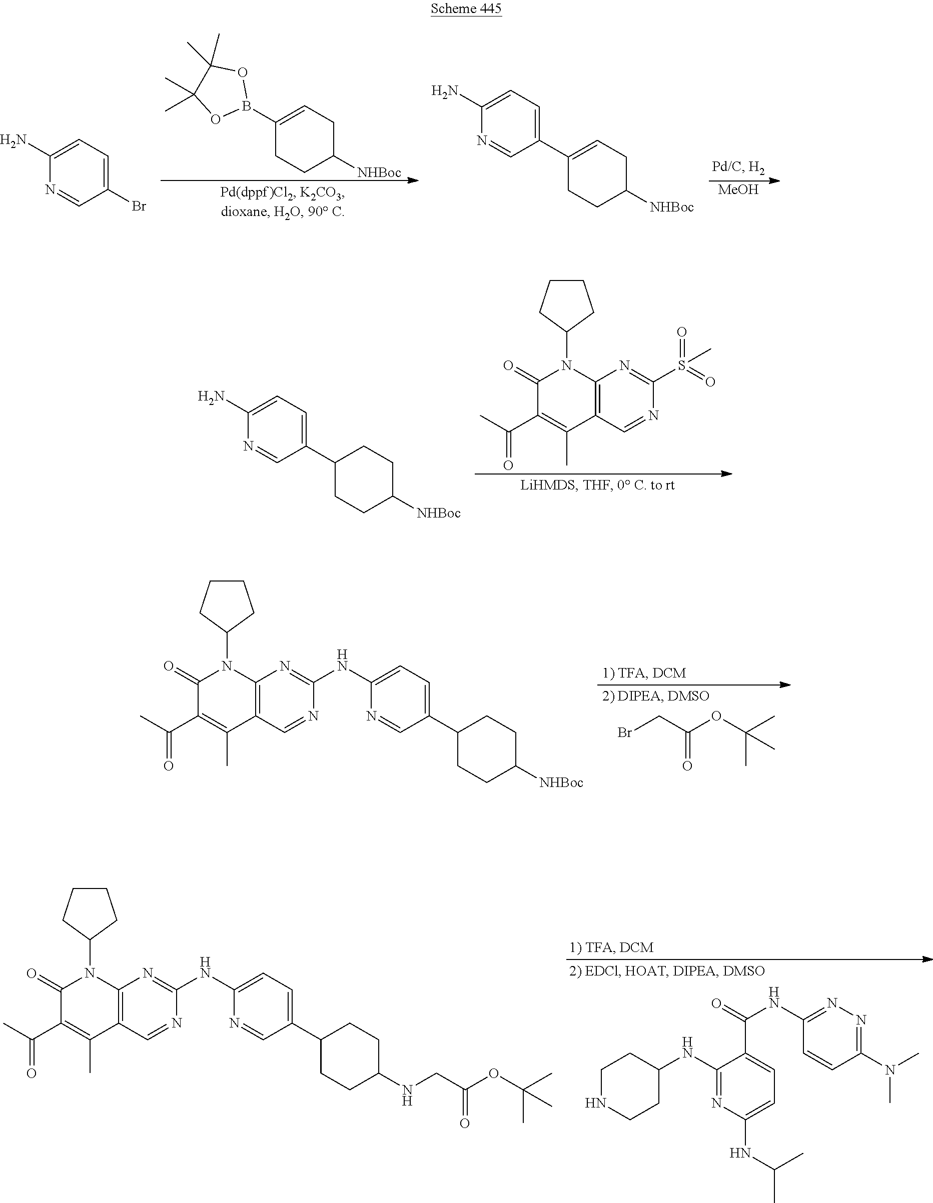

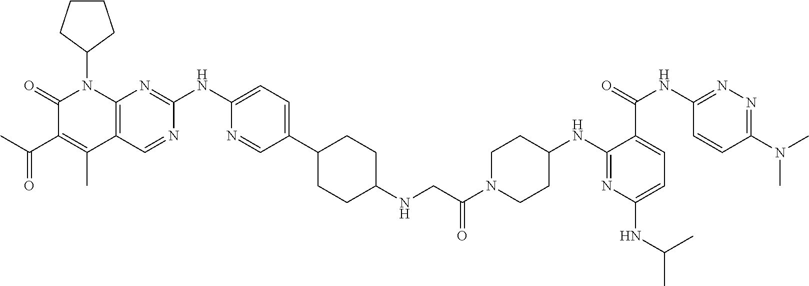 [SMILES: CC(=O)c1c(C)c2cnc(Nc3ccc(C4CCC(NCC(=O)N5CCC(Nc6nc(NC(C)C)ccc6C(=O)Nc6ccc(N(C)C)nn6)CC5)CC4)cn3)nc2n(C2CCCC2)c1=O]

### Step 1. Synthesis of tert-butyl (4-(6-aminopyridin-3-yl)cyclohex-3-en-1-yl)carbamate

A mixture of 5-bromopyridin-2-amine (400 mg, 2.31 mmol) and tert-butyl (4-(4,4,5,5-tetramethyl-1,3,2-dioxaborolan-2-yl)cyclohex-3-en-1-yl)carbamate (747 mg, 2.31 mmol) in dioxane (15 mL) and water (3 mL) were added K₂CO₃ (1.60 g, 11.56 mmol) and Pd(dppf)Cl₂ (169 mg, 0.23 mmol) at rt under N₂. After stirring at 90° C. for 4 h, the reaction mixture was poured into water (100 mL) and extracted with ethyl acetate (3×80 mL). The combined organic layers were washed with saturated brine (80 mL), dried over anhydrous sodium sulfate, filtered, and evaporated under reduced pressure. The resulting residue was purified by silica gel flash chromatography to give the desired product (600 mg, 89% yield) as a brown solid. MS (ESI) m/z=290.6 [M+H]⁺.

### Step 2. Synthesis of tert-butyl (4-(6-aminopyridin-3-yl)cyclohexyl)carbamate

A mixture of tert-butyl (4-(6-aminopyridin-3-yl)cyclohex-3-en-1-yl)carbamate (600 mg, 2.07 mmol) and 10% Pd/C (120 mg) in MeOH (20 mL) was stirred at rt for 8 h under hydrogen atmosphere. The mixture was filtered and concentrated to provide the title compound (500 mg, 83% yield) as a white solid. MS (ESI) m/z=292.7 [M+H]⁺.

### Step 3. Synthesis of tert-butyl (4-(6-((6-acetyl-8-cyclopentyl-5-methyl-7-oxo-7,8-dihydropyrido[2,3-d]pyrimidin-2-yl)amino)pyridin-3-yl)cyclohexyl)carbamate

To a solution of tert-butyl (4-(6-aminopyridin-3-yl)cyclohexyl)carbamate (100 mg, 0.34 mmol) and 6-acetyl-8-cyclopentyl-5-methyl-2-(methylsulfonyl)pyrido[2,3-d]pyrimidin-7(8H)-one (179 mg, 0.51 mmol) in THF (10 mL) was added LiHMDS (172 mg, 1.03 mmol) dropwise at 0° C. The mixture was stirred at rt for 2 h, before it was poured into water (30 mL) and extracted with ethyl acetate (2×30 mL). The combined organic layers were washed with saturated brine (30 mL), dried over anhydrous sodium sulfate, filtered, and evaporated under reduced pressure. The residue was purified by silica gel chromatography to provide the desired product (25 mg, 13% yield) as a light-yellow solid. MS(ESI) m/z=561.7 [M+H]⁺.

The remaining steps were performed according to the procedures for preparing CPD-373 to provide the desired product (3.0 mg, 7% yield over 4 steps) as a yellow solid. MS (ESI) m/z=899.8 [M+H]⁺.

### Example 446. 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)piperidin-4-yl)amino)-N-(6-(dimethylamino)pyridazin-3-yl)-6-(isopropylamino)nicotinamide (CPD-390)

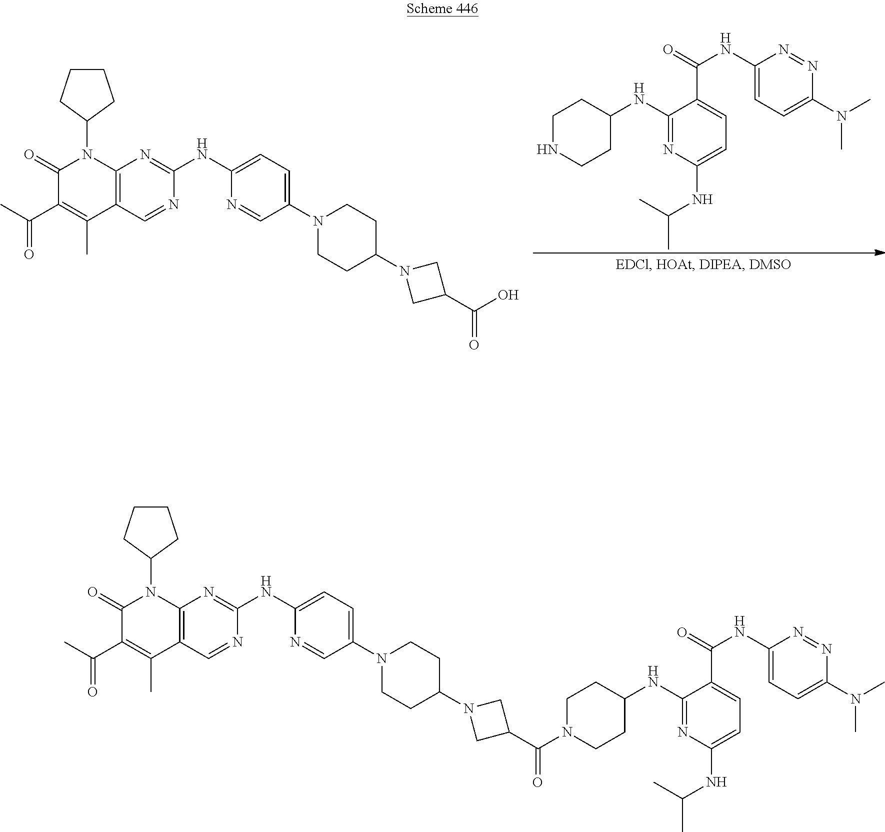 [SMILES: CC(=O)c1c(C)c2cnc(Nc3ccc(N4CCC(N5CC(C(=O)O)C5)CC4)cn3)nc2n(C2CCCC2)c1=O ; CC(C)Nc1ccc(C(=O)Nc2ccc(N(C)C)nn2)c(NC2CCNCC2)n1 ; CC(=O)c1c(C)c2cnc(Nc3ccc(N4CCC(N5CC(C(=O)N6CCC(Nc7nc(NC(C)C)ccc7C(=O)Nc7ccc(N(C)C)nn7)CC6)C5)CC4)cn3)nc2n(C2CCCC2)c1=O]

CPD-390 was synthesized following the standard procedure for preparing CPD-143 (4.6 mg, 5400 yield) as a yellow solid. MS (ESI) m/z=926.6 [M+H]⁺.

### Example 447. 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carbonyl)-2-azaspiro[3.3]heptan-6-yl)amino)-N-(6-(dimethylamino)pyridazin-3-yl)-6-(isopropylamino)nicotinamide (CPD-391)

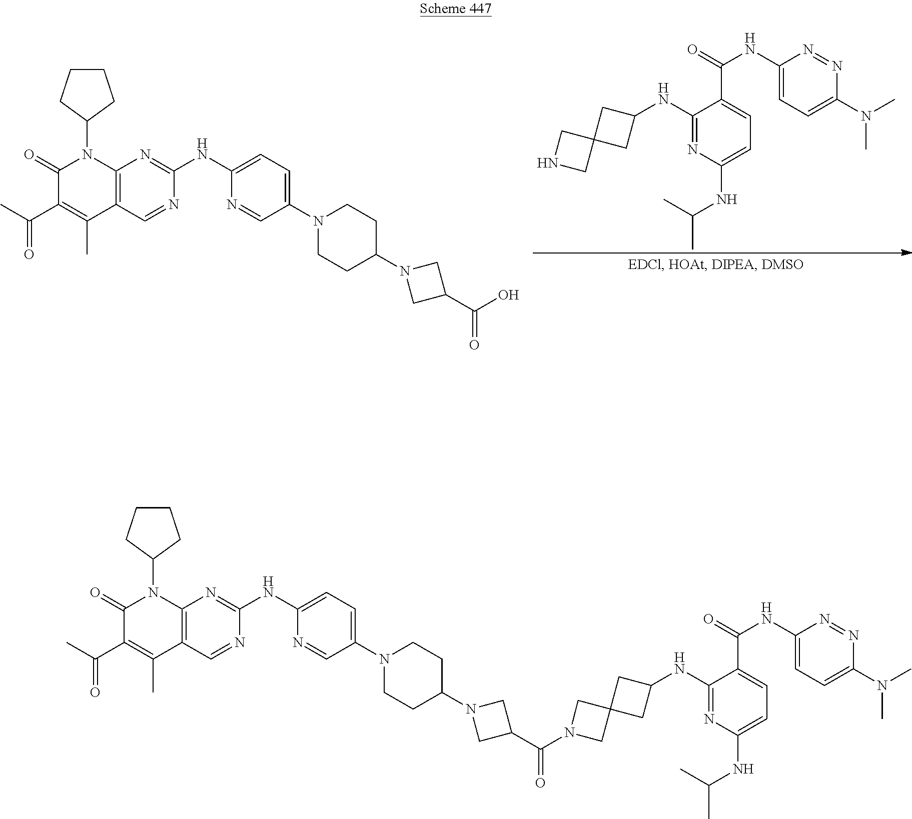 [SMILES: CC(=O)c1c(C)c2cnc(Nc3ccc(N4CCC(N5CC(C(=O)O)C5)CC4)cn3)nc2n(C2CCCC2)c1=O ; CC(C)Nc1ccc(C(=O)Nc2ccc(N(C)C)nn2)c(NC2CC3(CNC3)C2)n1 ; CC(=O)c1c(C)c2cnc(Nc3ccc(N4CCC(N5CC(C(=O)N6CC7(CC(Nc8nc(NC(C)C)ccc8C(=O)Nc8ccc(N(C)C)nn8)C7)C6)C5)CC4)cn3)nc2n(C2CCCC2)c1=O]

CPD-391 was synthesized following the standard procedure for preparing CPD-143 (3.9 mg, 45% yield) as a yellow solid. MS (ESI) m/z=938.5 [M+H]⁺.

### Example 448. 2-((1-(2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetyl)piperidin-4-yl)amino)-N-(6-(dimethylamino)pyridazin-3-yl)-6-(isopropylamino)nicotinamide (CPD-392)

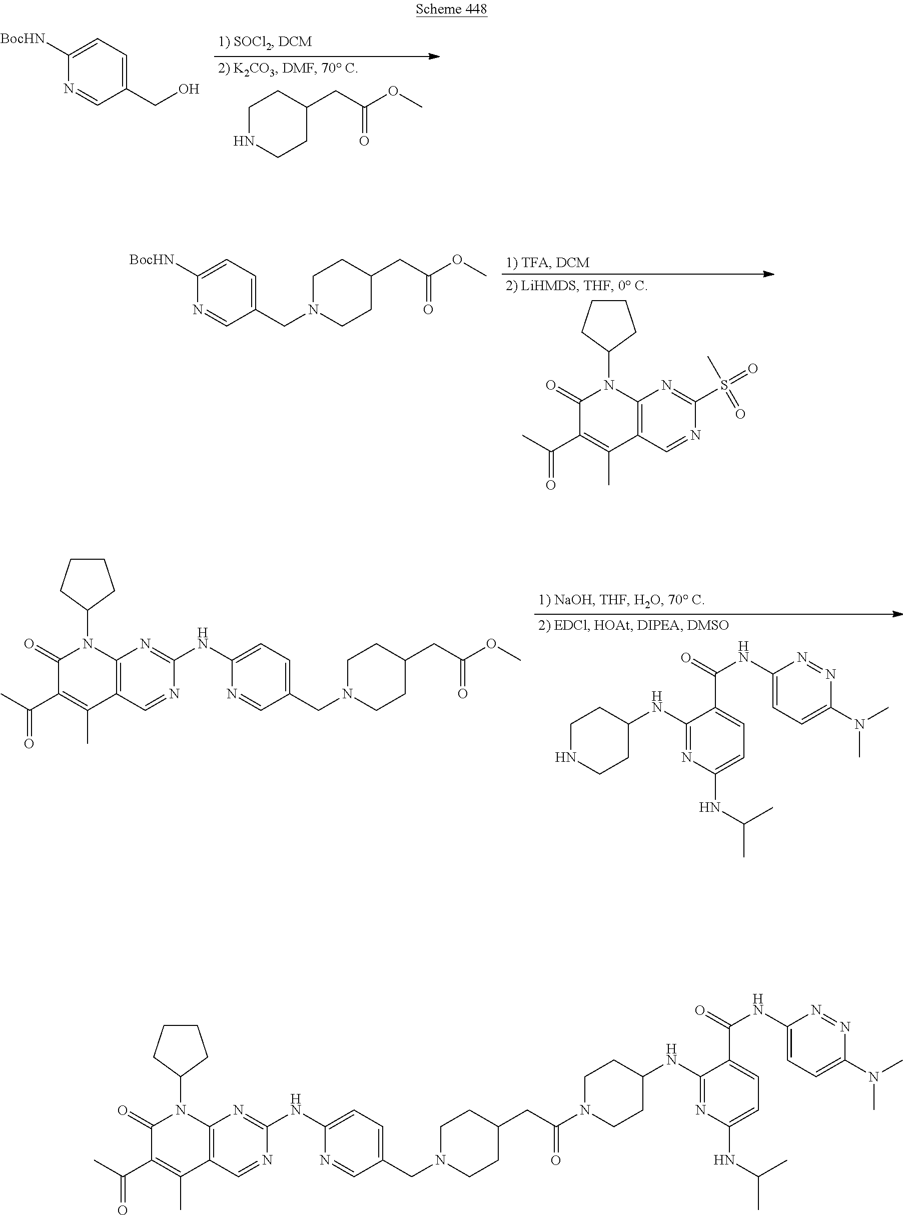

### Step 1. Synthesis of tert-butyl (5-(chloromethyl)pyridin-2-yl)carbamate

To a solution of tert-butyl (5-(hydroxymethyl)pyridin-2-yl)carbamate (130 mg, 0.6 mmol) in dichloromethane (2 mL) was added thionyl chloride (700 mg, 5.8 mmol). The mixture was stirred at rt for 1 h. The mixture was concentrated to give the desired product (140 mg, 99% yield) as a yellow oil. This compound was used directly in the next step without further purification. MS (ESI) m/z-245.1 [M+H]⁺.

### Step 2. Synthesis of methyl 2-(1-((6-((tert-butoxycarbonyl)amino)pyridin-3-yl)methyl)piperidin-4-yl)acetate

To a mixture of tert-butyl (5-(chloromethyl)pyridin-2-yl)carbamate (130 mg, 0.5 mmol) and methyl 2-(piperidin-4-yl)acetate (217 mg, 0.8 mmol) in DMF (5 mL) was added K₂CO₃ (222 mg, 1.6 mmol) at room temperature. The reaction mixture was stirred at 70° C. for 16 h. The reaction mixture was poured into water (20 mL) and extracted with ethyl acetate (3×20 mL). The combined organic layers were washed with saturated brine (20 mL), dried over anhydrous Na₂SO₄, filtered and concentrated. The resulting residue was purified by silica gel flash chromatography to give the desired product (170 mg, 87% yield) as a white solid. MS (ESI) m/z=364.7 [M+H]⁺.

### Step 3. Synthesis of methyl 2-(1-((6-aminopyridin-3-yl)methyl)piperidin-4-yl)acetate

To a solution of methyl 2-(1-((6-((tert-butoxycarbonyl)amino)pyridin-3-yl)methyl)piperidin-4-yl)acetate (170 mg, 0.46 mmol) in dichloromethane (2 mL) was added TFA (2 mL). The mixture was stirred at rt for 1 h. The mixture was concentrated and purified by reverse-phase chromatography to give the desired product (106 mg, 60% yield) as a white solid. MS (ESI) m/z=264.3 [M+H]⁺.

### Step 4. Synthesis of methyl 2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetate

To a mixture of methyl 2-(1-((6-aminopyridin-3-yl)methyl)piperidin-4-yl)acetate (50 mg, 0.18 mmol) and 6-acetyl-8-cyclopentyl-5-methyl-2-(methylsulfonyl)pyrido[2,3-d]pyrimidin-7(8H)-one (53 mg, 0.15 mmol) in THF (5 mL) was added LiHMDS (0.4 mL, 1M in THF) at 0° C. The reaction mixture was stirred rt for 2 h. The reaction mixture was poured into water (10 mL) and extracted with ethyl acetate (3×10 mL). The combined organic layers were washed with saturated brine (10 mL), dried over anhydrous Na₂SO₄, filtered and concentrated. The resulting residue was purified by silica gel flash chromatography to give the desired product (35 mg, 34% yield) as a yellow oil. MS (ESI) m/z=533.5 [M+H]⁺.

### Step 5. Synthesis of 2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetic acid

A mixture of methyl 2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetate (35 mg, 0.06 mmol) and sodium hydroxide (26 mg, 0.6 mmol) in THF (2 mL) and H₂O (1 mL) was stirred at 70° C. for 2 h. The reaction mixture was purified by reverse-phase chromatography to give the desired product (15 mg, 44% yield) as a white solid. MS (ESI) m/z=519.5 [M+H]⁺.

### Step 6. Synthesis of 2-((1-(2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetyl)piperidin-4-yl)amino)-N-(6-(dimethylamino)pyridazin-3-yl)-6-(isopropylamino)nicotinamide

A mixture of 2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetic acid (15 mg, 0.03 mmol), N-(6-(dimethylamino)pyridazin-3-yl)-6-(isopropylamino)-2-(piperidin-4-ylamino)nicotinamide (12 mg, 0.03 mmol), EDCI (8.3 mg, 0.04 mmol), HOAt (5.9 mg, 0.04 mmol) and DIPEA (18 mg, 0.14 mmol) in DMSO (2 mL) was stirred at rt for 16 h. The reaction mixture was purified by reverse-phase chromatography to give the desired product (3.4 mg, 11% yield) as a yellow solid. MS (ESI) m/z=899.7 [M+H]⁺.

### Example 449. 2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)azetidine-3-carboxamido)spiro[3.3]heptan-2-yl)amino)-N-(6-(dimethylamino)pyridazin-3-yl)-6-(isopropylamino)nicotinamide (CPD-393)

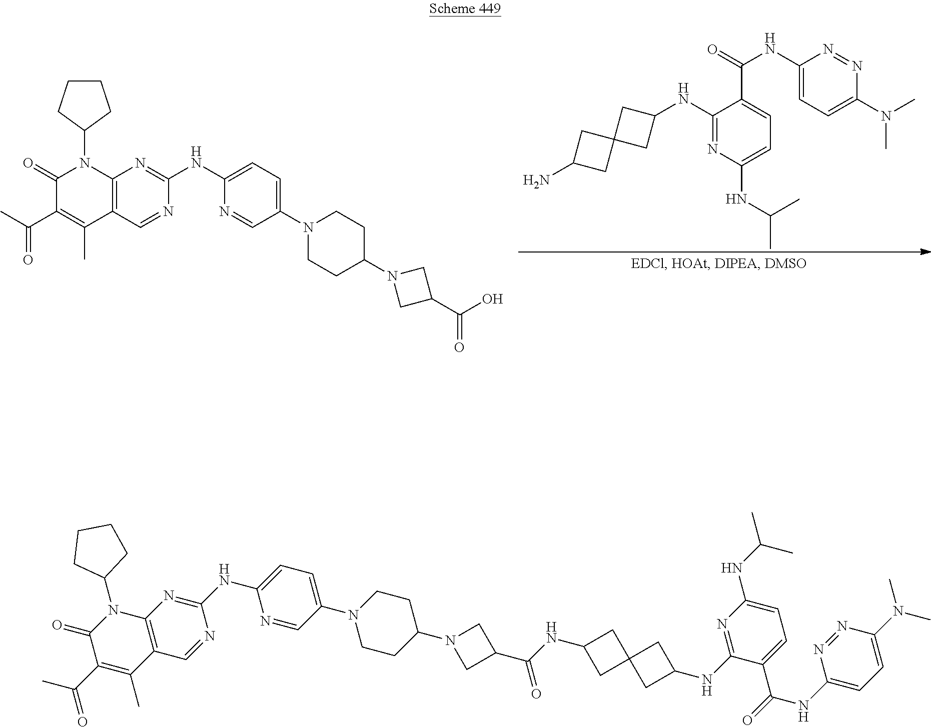 [SMILES: CC(=O)c1c(C)c2cnc(Nc3ccc(N4CCC(N5CC(C(=O)O)C5)CC4)cn3)nc2n(C2CCCC2)c1=O ; CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CCC(N5CC(C(=O)NC6CC7(C6)CC(Nc6nc(NC(C)C)ccc6C(=O)Nc6ccc(N(C)C)nn6)C7)C5)CC4)cn3)nc2n(C2CCCC2)c1=O]

CPD-393 was synthesized following the standard procedure for preparing CPD-143 (4.4 mg, 50% yield) as a yellow solid. MS (ESI) n/z=952.8 [M+H]⁺.

### Example 450. 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)azetidin-3-yl)amino)-N-(6-(dimethylamino)pyridazin-3-yl)-6-(isopropylamino)nicotinamide (CPD-394)

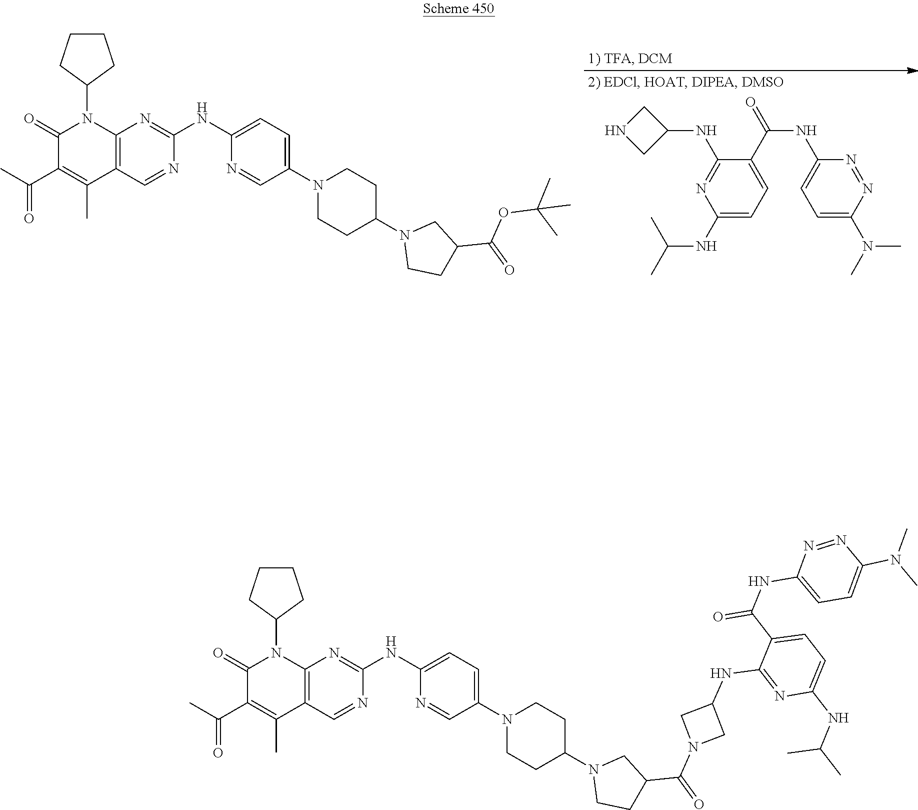

CPD-394 was synthesized following the standard procedures for preparing CPD-144 (2.6 mg, 3% yield over 2 steps) as a yellow solid. MS (ESI) m/z=912.8 [M+H]⁺.

### Example 451. 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)pyrrolidin-3-yl)amino)-N-(6-(dimethylamino)pyridazin-3-yl)-6-(isopropylamino)nicotinamide (CPD-395)

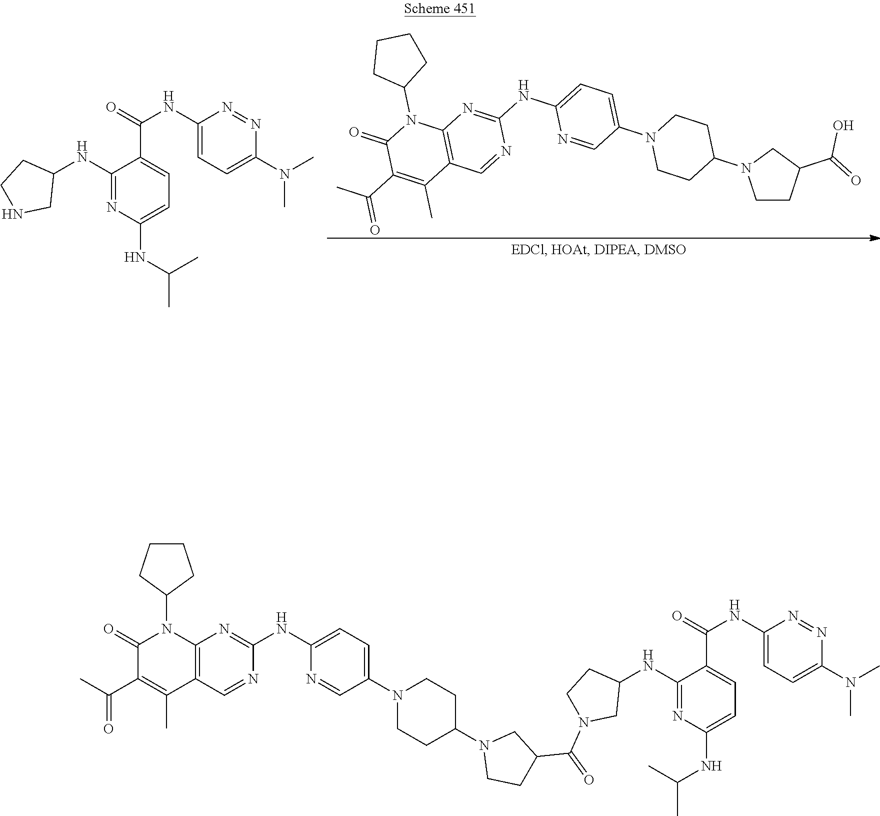

CPD-395 was synthesized following the standard procedure for preparing CPD-143 (2.7 mg, 33% yield) as a yellow solid. MS (ESI) m/z=926.7 [M+H]⁺.

### Example 452. 2-((1-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)piperidin-4-yl)amino)-N-(6-(dimethylamino)pyridazin-3-yl)-6-(isopropylamino)nicotinamide (CPD-396)

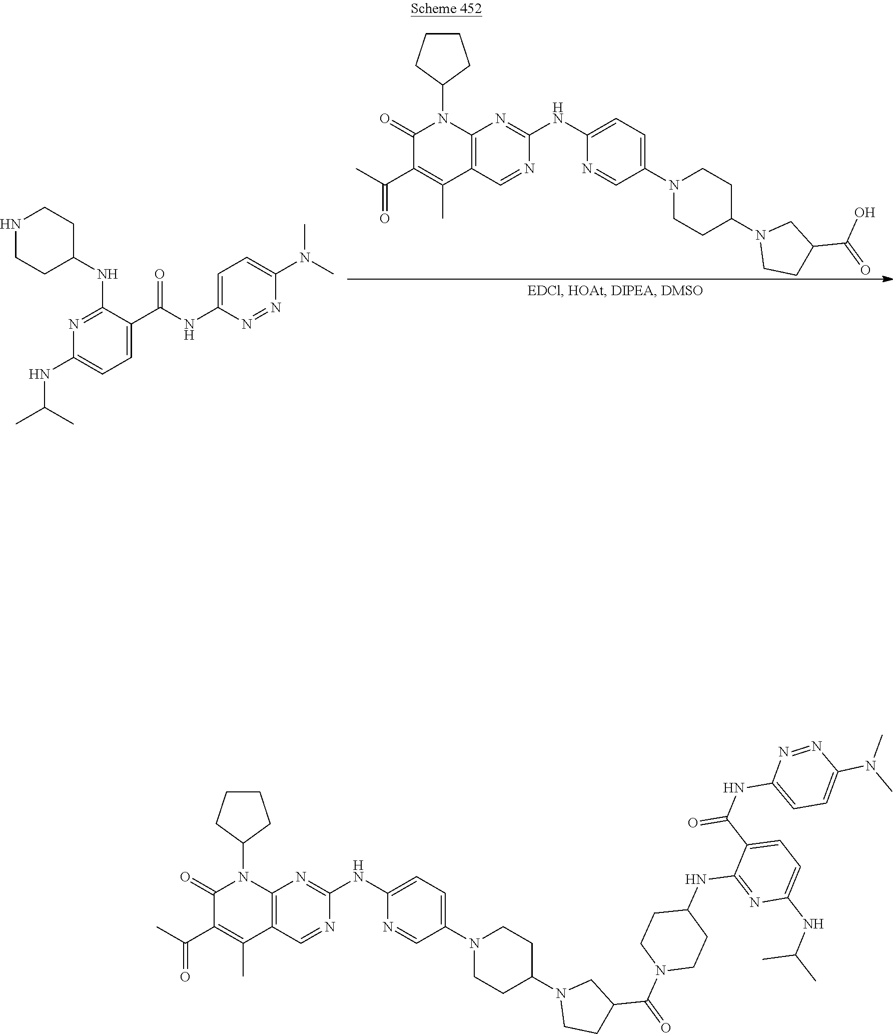 [SMILES: CC(C)Nc1ccc(C(=O)Nc2ccc(N(C)C)nn2)c(NC2CCNCC2)n1 ; CC(=O)c1c(C)c2cnc(Nc3ccc(N4CCC(N5CCC(C(=O)O)C5)CC4)cn3)nc2n(C2CCCC2)c1=O ; CC(=O)c1c(C)c2cnc(Nc3ccc(N4CCC(N5CCC(C(=O)N6CCC(Nc7nc(NC(C)C)ccc7C(=O)Nc7ccc(N(C)C)nn7)CC6)C5)CC4)cn3)nc2n(C2CCCC2)c1=O]

CPD-396 was synthesized following the standard procedure for preparing CPD-143 (2.2 mg, 26% yield) as a yellow solid. MS (ESI) m/z=940.7 [M+H]⁺.

### Example 453. 2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)spiro[3.3]heptan-2-yl)amino)-N-(6-(dimethylamino)pyridazin-3-yl)-6-(isopropylamino)nicotinamide (CPD-397)

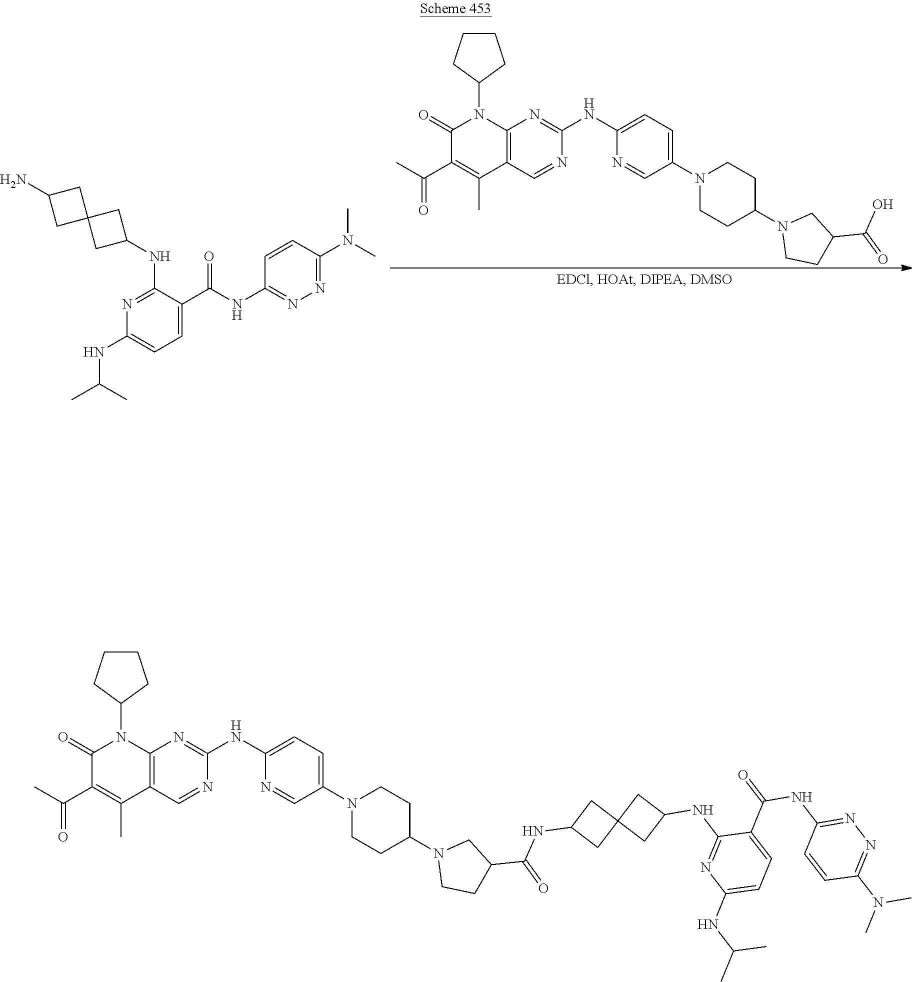 [SMILES: CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CCC(N5CCC(C(=O)O)C5)CC4)cn3)nc2n(C2CCCC2)c1=O ; CC(=O)c1c(C)c2cnc(Nc3ccc(N4CCC(N5CCC(C(=O)NC6CC7(C6)CC(Nc6nc(NC(C)C)ccc6C(=O)Nc6ccc(N(C)C)nn6)C7)C5)CC4)cn3)nc2n(C2CCCC2)c1=O]

CPD-397 was synthesized following the standard procedure for preparing CPD-143 (0.8 mg, 15% yield) as a yellow solid. MS (ESI) m/z=966.8 [M+H]⁺.

### Example 454. 2-((2-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carbonyl)-2-azaspiro[3.3]heptan-6-yl)amino)-N-(6-(dimethylamino)pyridazin-3-yl)-6-(isopropylamino)nicotinamide (CPD-398)

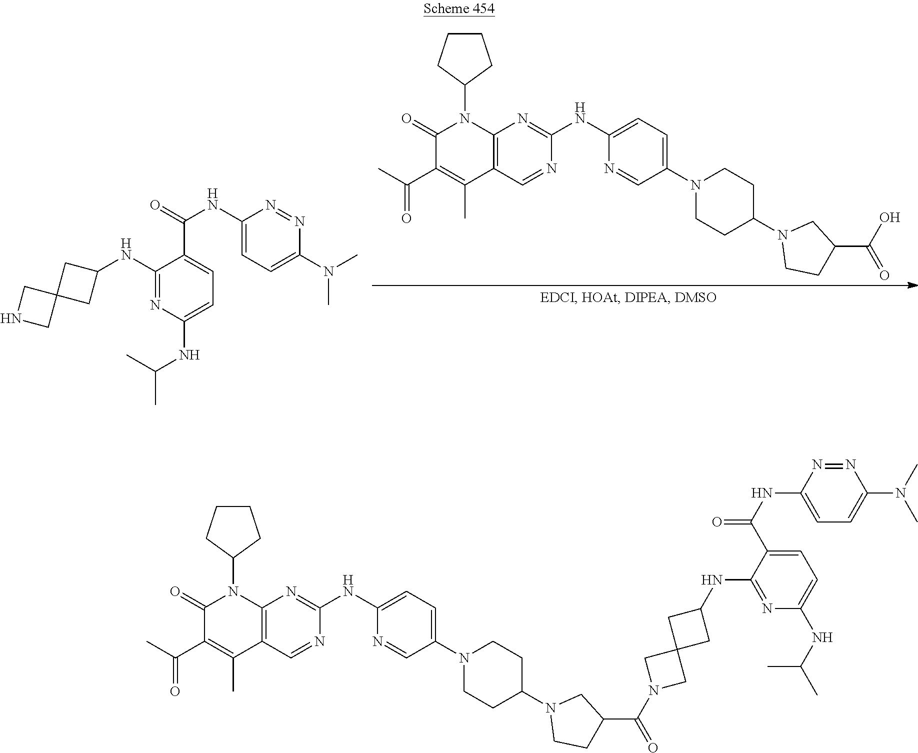 [SMILES: CC(C)Nc1ccc(C(=O)Nc2ccc(N(C)C)nn2)c(NC2CC3(CNC3)C2)n1 ; CC(=O)c1c(C)c2cnc(Nc3ccc(N4CCC(N5CCC(C(=O)O)C5)CC4)cn3)nc2n(C2CCCC2)c1=O ; CC(=O)c1c(C)c2cnc(Nc3ccc(N4CCC(N5CCC(C(=O)N6CC7(CC(Nc8nc(NC(C)C)ccc8C(=O)Nc8ccc(N(C)C)nn8)C7)C6)C5)CC4)cn3)nc2n(C2CCCC2)c1=O]

CPD-398 was synthesized following the standard procedure for preparing CPD-143 (2.9 mg, 34% yield) as a yellow solid. MS (ESI) m/z=952.7 [M+H]⁺.

### Example 455. 2-((−4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide (CPD-399)

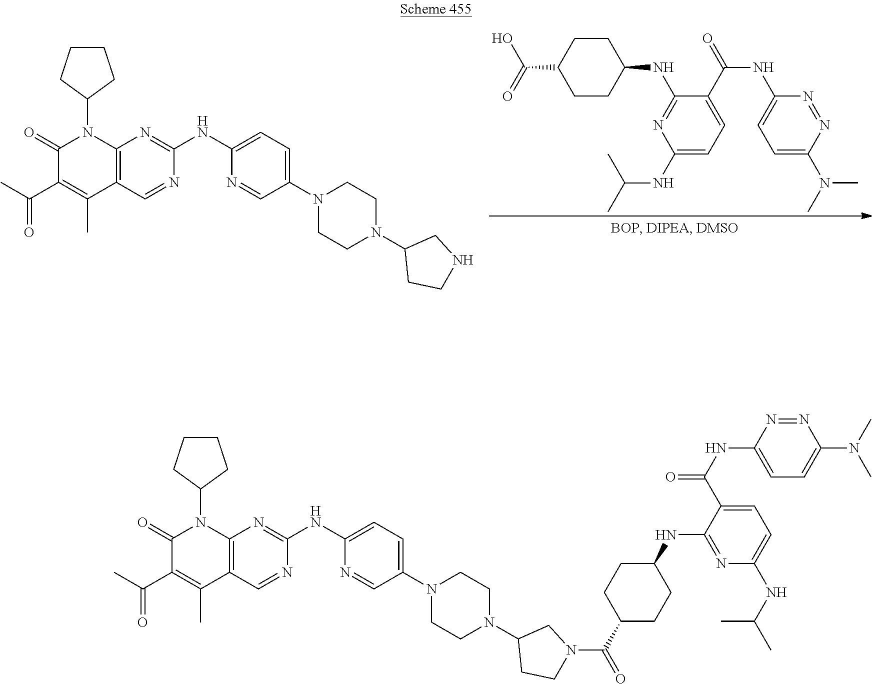

CPD-399 was synthesized following the standard procedure for preparing CPD-261 (10.0 mg, 27% yield) as a yellow solid. MS (ESI) m/z=940.7 [M+H]⁺.

### Example 456. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)spiro[3.3]heptan-2-yl)amino)-N-(6-(dimethylamino)pyridazin-3-yl)-6-(isopropylamino)nicotinamide (CPD-400)

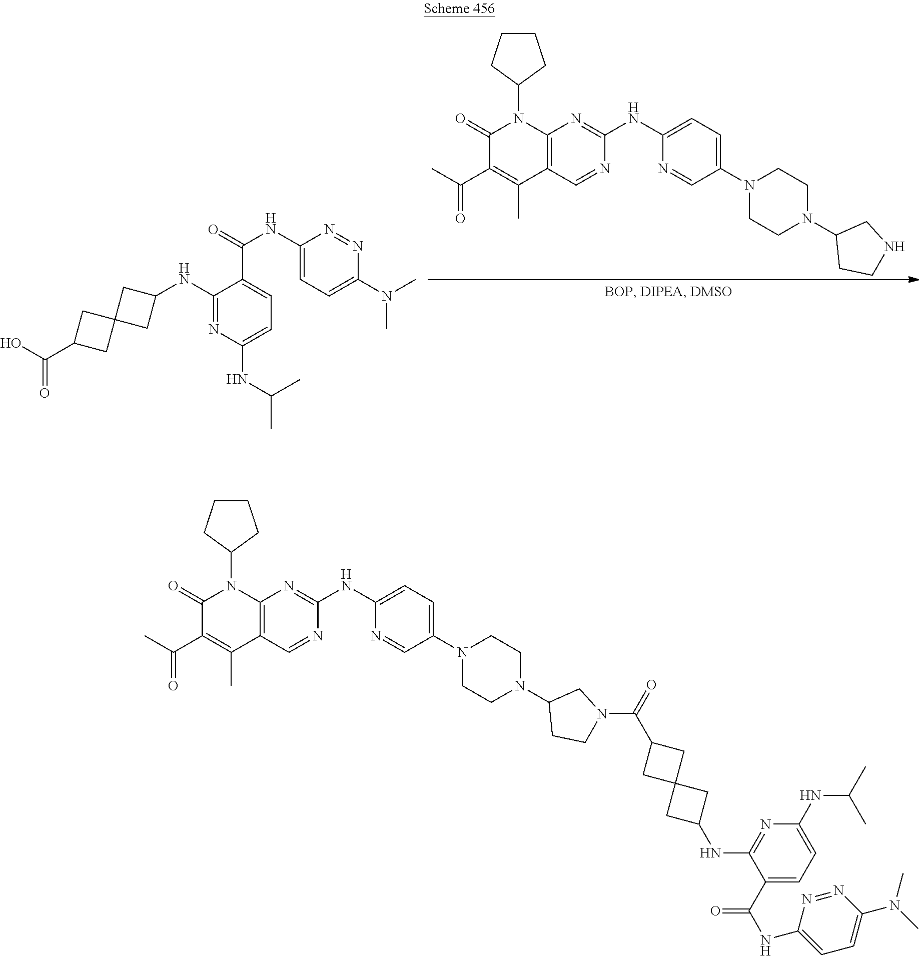 [SMILES: CC(C)Nc1ccc(C(=O)Nc2ccc(N(C)C)nn2)c(NC2CC3(C2)CC(C(=O)O)C3)n1 ; CC(=O)c1c(C)c2cnc(Nc3ccc(N4CCN(C5CCNC5)CC4)cn3)nc2n(C2CCCC2)c1=O ; CC(=O)c1c(C)c2cnc(Nc3ccc(N4CCN(C5CCN(C(=O)C6CC7(CC(Nc8nc(NC(C)C)ccc8C(=O)Nc8ccc(N(C)C)nn8)C7)C6)C5)CC4)cn3)nc2n(C2CCCC2)c1=O]

CPD-400 was synthesized following the standard procedure for preparing CPD-261 (9.0 mg, 33% yield) as a yellow solid. MS (ESI) m/z=952.8 [M+H]⁺.

### Example 457. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)azetidine-1-carbonyl)spiro[3.3]heptan-2-yl)amino)-N-(6-(dimethylamino)pyridazin-3-yl)-6-(isopropylamino)nicotinamide (CPD-401)

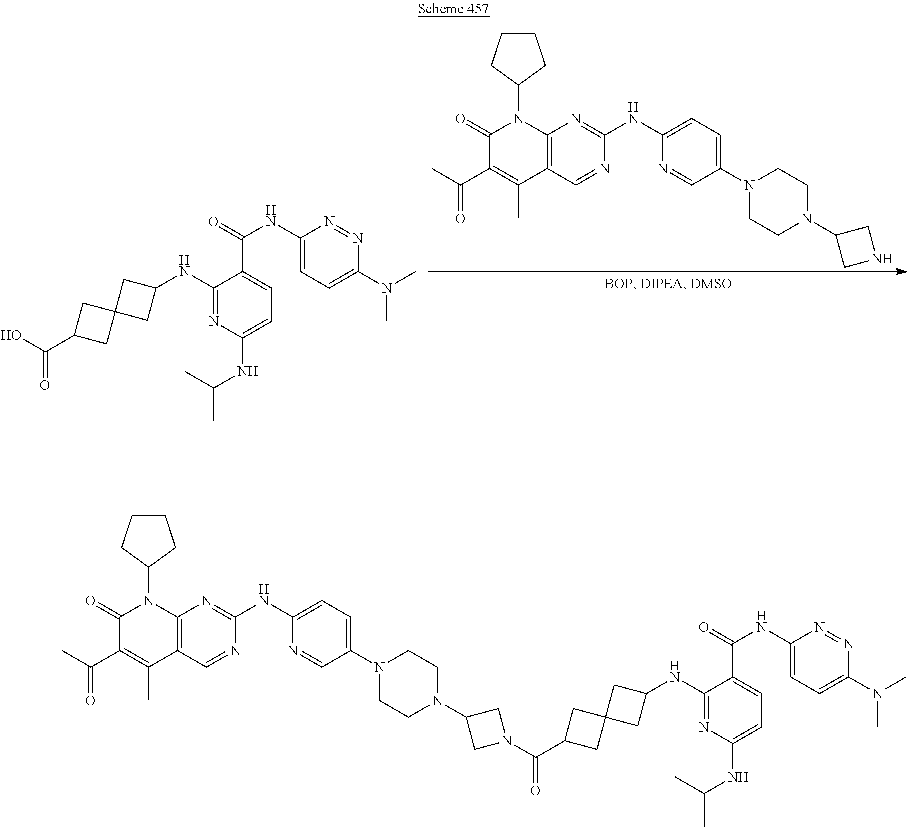 [SMILES: CC(C)Nc1ccc(C(=O)Nc2ccc(N(C)C)nn2)c(NC2CC3(C2)CC(C(=O)O)C3)n1 ; CC(=O)c1c(C)c2cnc(Nc3ccc(N4CCN(C5CNC5)CC4)cn3)nc2n(C2CCCC2)c1=O ; CC(=O)c1c(C)c2cnc(Nc3ccc(N4CCN(C5CN(C(=O)C6CC7(CC(Nc8nc(NC(C)C)ccc8C(=O)Nc8ccc(N(C)C)nn8)C7)C6)C5)CC4)cn3)nc2n(C2CCCC2)c1=O]

CPD-401 was synthesized following the standard procedure for preparing CPD-261 (8.0 mg, 14% yield) as a yellow solid. MS (ESI) m/z=938.7 [M+H]⁺.

### Example 458. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-(isopropylamino)nicotinamide (CPD-402)

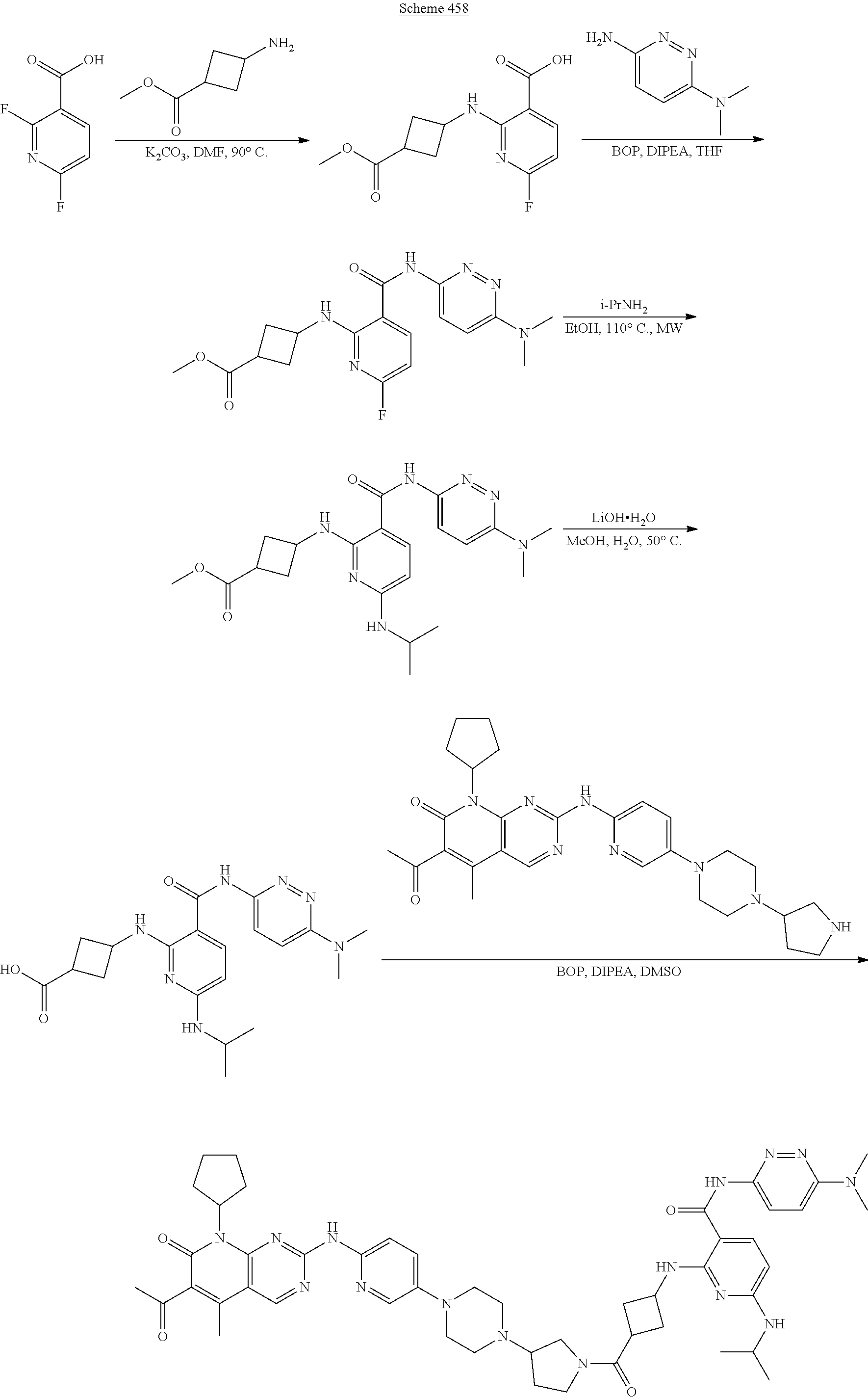

CPD-402 was synthesized following the standard procedures for preparing CPD-82 (10.0 mg, 6% yield over 5 steps) as a yellow solid. MS (ESI) m/z=912.7 [M+H]⁺.

### Example 459. 2-(((1R,3S)-3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclopentyl)amino)-N-(6-(dimethylamino)pyridazin-3-yl)-6-(isopropylamino)nicotinamide (CPD-403)

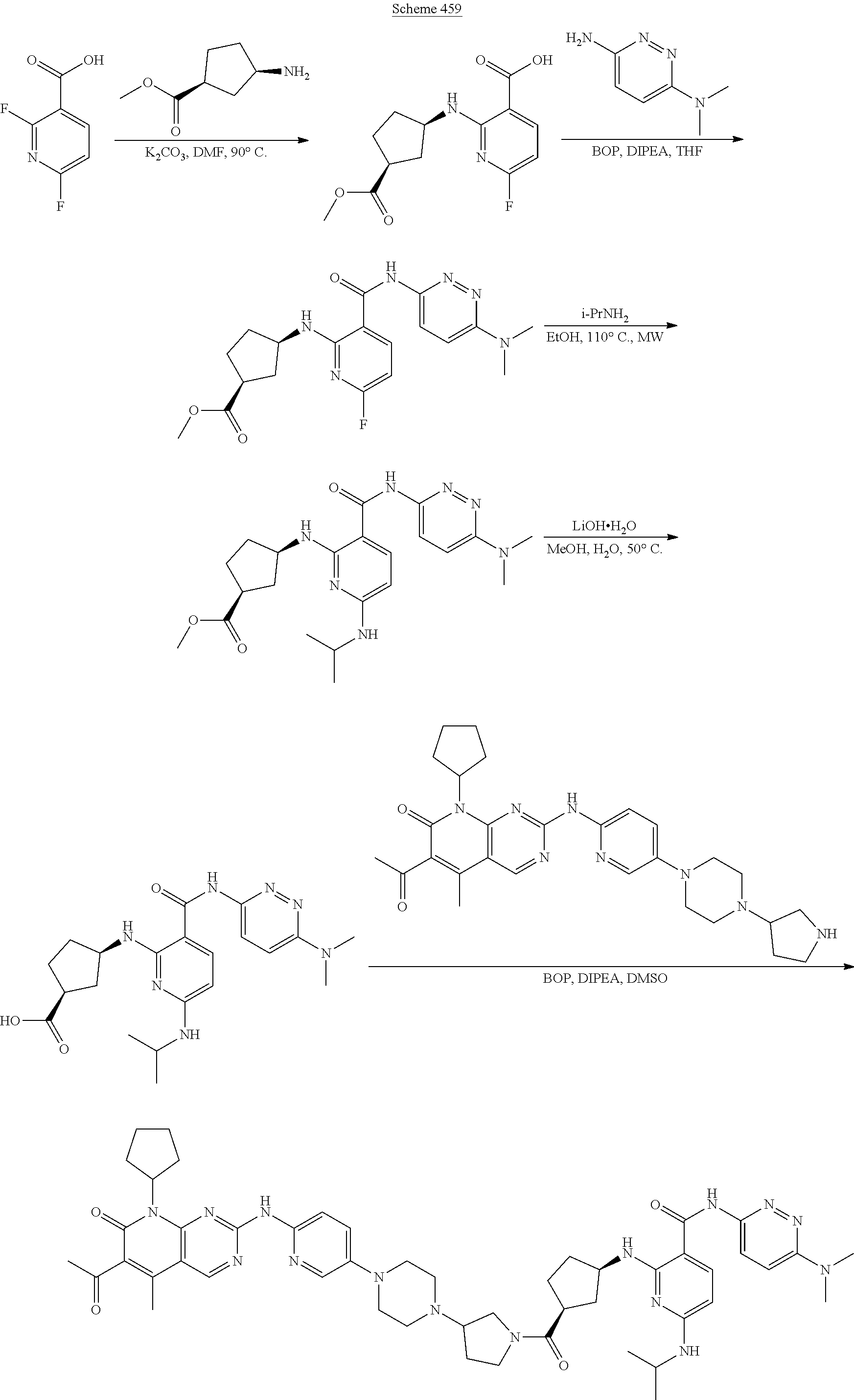

CPD-403 was synthesized following the standard procedures for preparing CPD-82 (10.0 mg, 8% yield over 5 steps) as a yellow solid. MS (ESI) m/z=926.7 [M+H]⁺.

### Example 460. 2-((2-(3-(4-(6-((6-acetyl-8-cyclopentyl-5-methyl-7-oxo-7,8-dihydropyrido[2,3-d]pyrimidin-2-yl)(methyl)amino)pyridin-3-yl)piperazin-1-yl)propanoyl)-2-azaspiro[3.3]heptan-6-yl)amino)-N-(1,5-dimethyl-1H-pyrazol-3-yl)-6-(isopropylamino)nicotinamide (CPD-404)

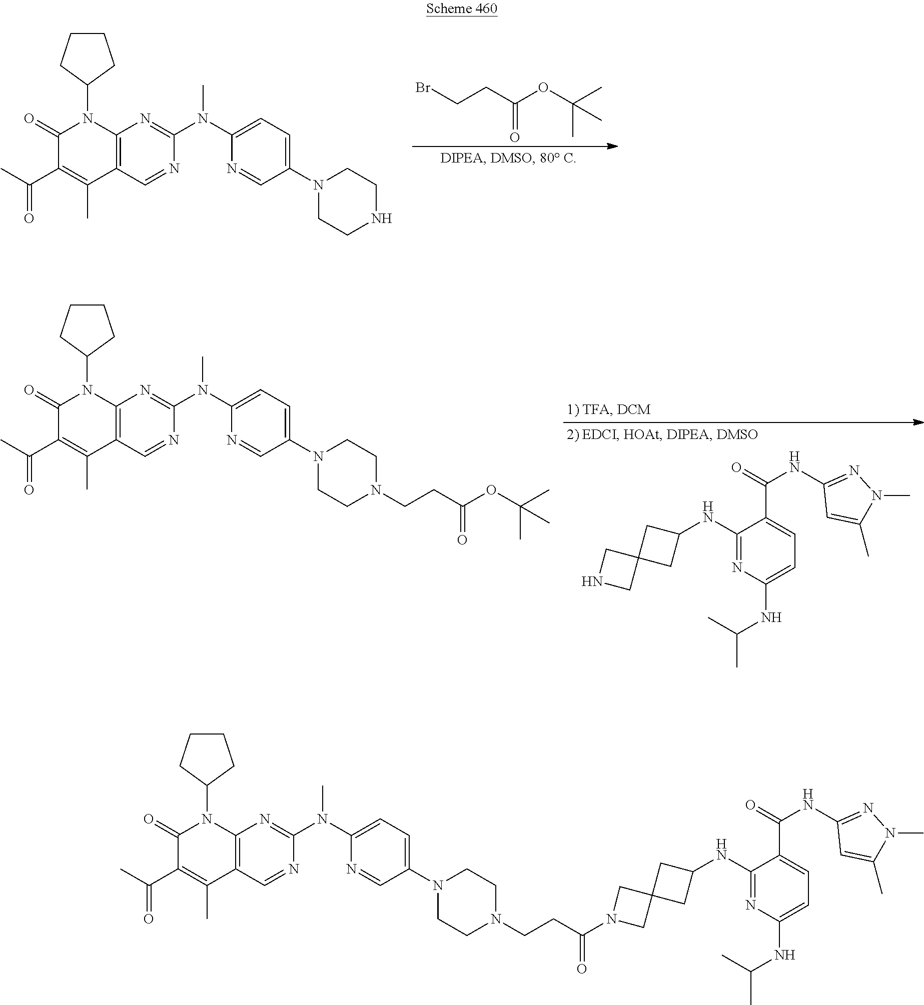

CPD-404 was synthesized following the standard procedures for preparing CPD-373 (25.0 mg, 23% yield over 3 steps) as a yellow solid. MS (ESI) m/z=899.8 [M+H]⁺.

### Example 461. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(2-cyclopropyl-1-methyl-1H-imidazol-4-yl)-6-((methyl-d₃)amino)nicotinamide (CPD-405)

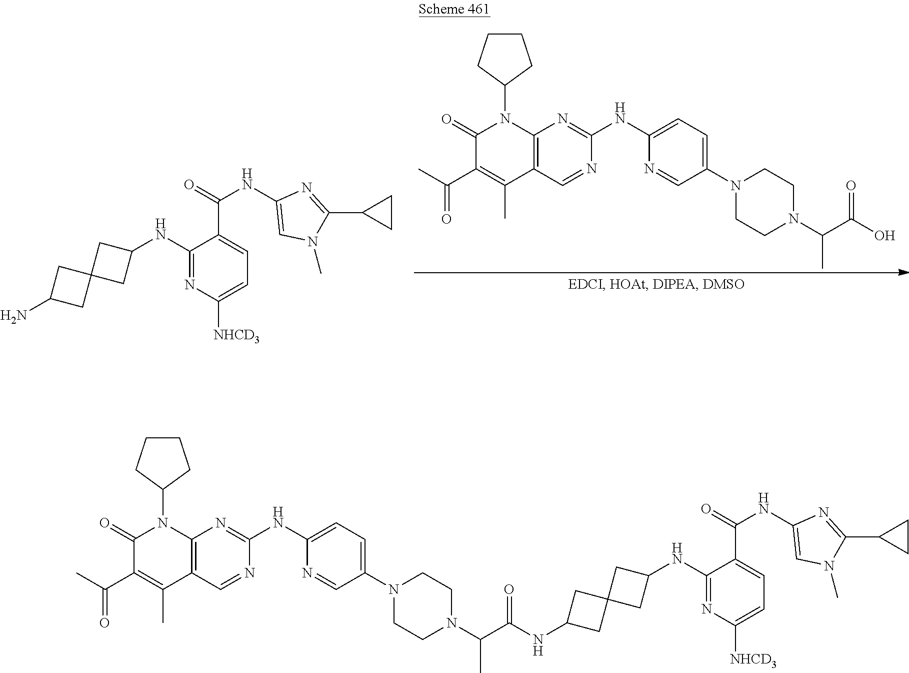

CPD-405 was synthesized following the standard procedure for preparing CPD-143 (2.0 mg, 12% yield) as a yellow solid. MS (ESI) m/z=900.6 [M+H]⁺.

### Example 462. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(6-(dimethylamino)pyridazin-3-yl)-6-(isopropylamino)nicotinamide (CPD-406)

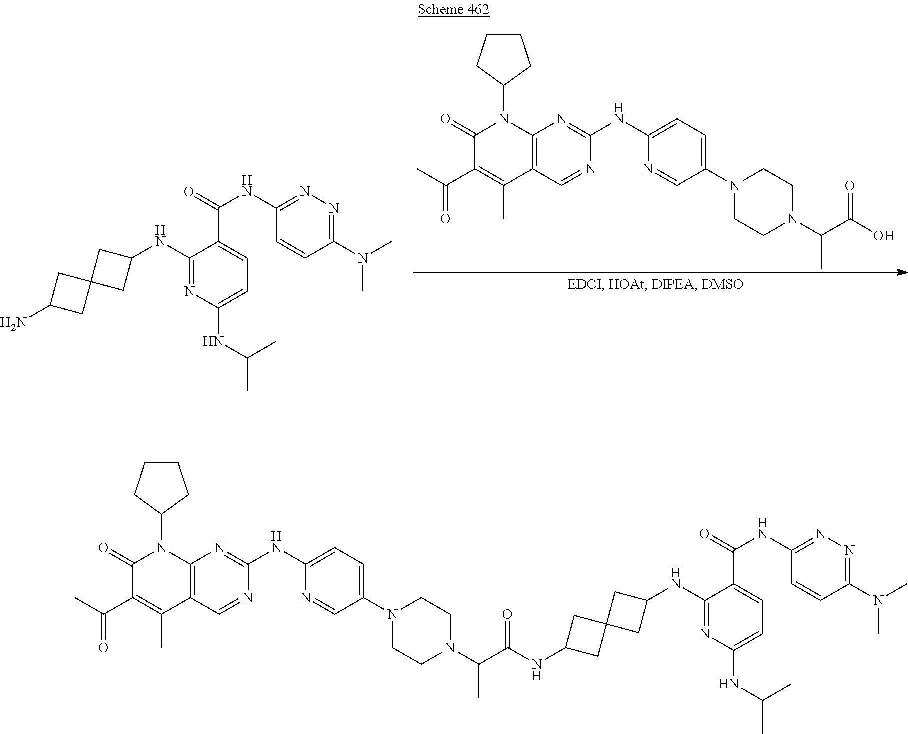 [SMILES: CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CCN(C(C)C(=O)O)CC4)cn3)nc2n(C2CCCC2)c1=O ; CC(=O)c1c(C)c2cnc(Nc3ccc(N4CCN(C(C)C(=O)NC5CC6(C5)CC(Nc5nc(NC(C)C)ccc5C(=O)Nc5ccc(N(C)C)nn5)C6)CC4)cn3)nc2n(C2CCCC2)c1=O]

CPD-406 was synthesized following the standard procedure for preparing CPD-143 (1.2 mg, 4% yield) as a yellow solid. MS (ESI) m/z=926.7 [M+H]⁺.

### Example 463. 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(6-(dimethylamino)pyridazin-3-yl)-6-(isopropylamino)nicotinamide (CPD-407)

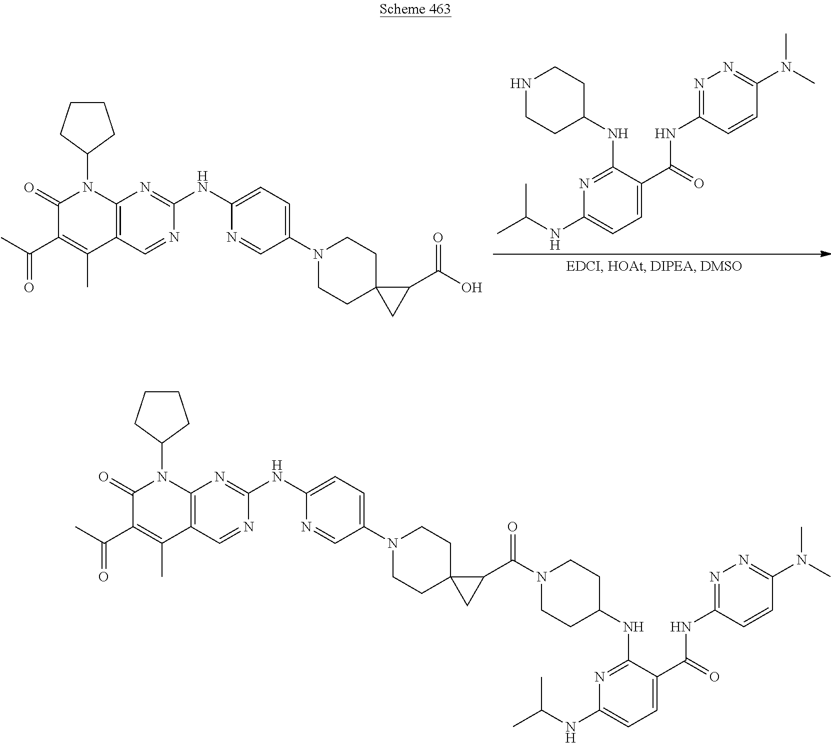 [SMILES: CC(=O)c1c(C)c2cnc(Nc3ccc(N4CCC5(CC4)CC5C(=O)O)cn3)nc2n(C2CCCC2)c1=O ; CC(C)Nc1ccc(C(=O)Nc2ccc(N(C)C)nn2)c(NC2CCNCC2)n1 ; CC(=O)c1c(C)c2cnc(Nc3ccc(N4CCC5(CC4)CC5C(=O)N4CCC(Nc5nc(NC(C)C)ccc5C(=O)Nc5ccc(N(C)C)nn5)CC4)cn3)nc2n(C2CCCC2)c1=O]

CPD-407 was synthesized following the standard procedure for preparing CPD-143 (8.6 mg, 44% yield) as a yellow solid. MS (ESI) m/z=897.7 [M+H]⁺.

### Example 464. 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(1,5-dimethyl-1H-pyrazol-3-yl)-6-(isopropylamino)nicotinamide (CPD-408)

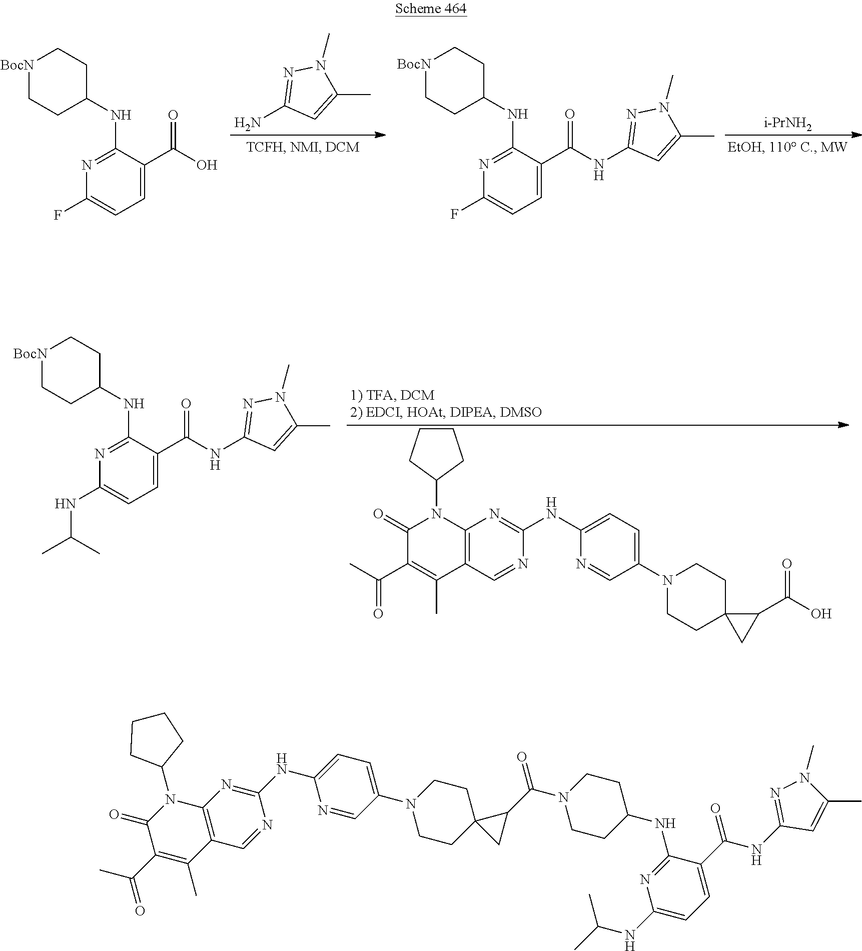

CPD-408 was synthesized following the standard procedures for preparing CPD-144 (9.8 mg, 28% yield over 4 steps) as a yellow solid. MS (ESI) m/z=870.6 [M+H]⁺.

### Example 465. 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-N-(2-cyclopropyl-1-menthyl-1H-imidazol-4-yl)-6-(isopropylamino)nicotinamide (CPD-409)

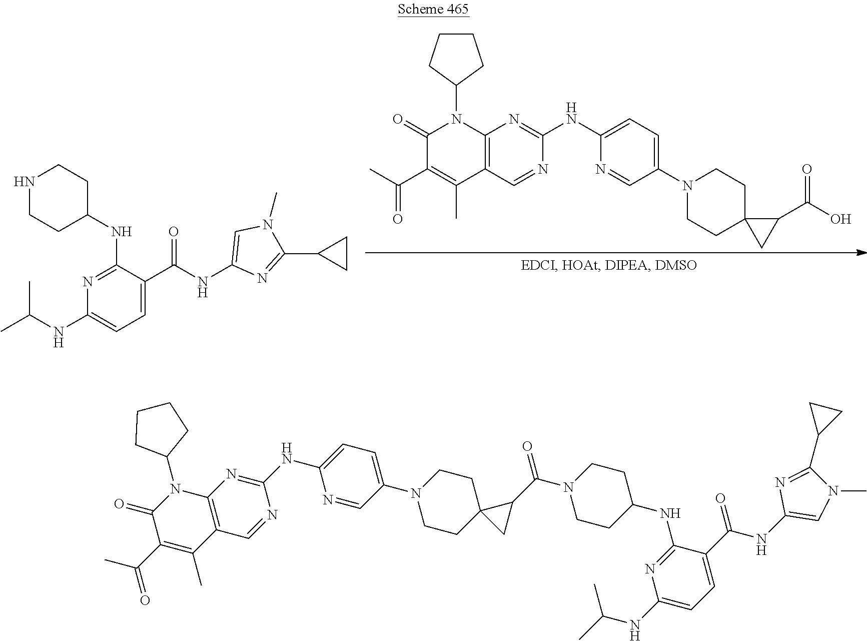 [SMILES: CC(C)Nc1ccc(C(=O)Nc2cn(C)c(C3CC3)n2)c(NC2CCNCC2)n1 ; CC(=O)c1c(C)c2cnc(Nc3ccc(N4CCC5(CC4)CC5C(=O)O)cn3)nc2n(C2CCCC2)c1=O ; CC(=O)c1c(C)c2cnc(Nc3ccc(N4CCC5(CC4)CC5C(=O)N4CCC(Nc5nc(NC(C)C)ccc5C(=O)Nc5cn(C)c(C6CC6)n5)CC4)cn3)nc2n(C2CCCC2)c1=O]

CPD-409 was synthesized following the standard procedures for preparing CPD-143 (17.4 mg, 48% yield) as a yellow solid. MS (ESI) m/z=896.8 [M+H]⁺.

### Example 466. 2-((3-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)propan-2-yl)cyclobutyl)amino)-6-(isopropylamino)-N-(5-methylisoxazol-3-yl)nicotinamide (CPD-410)

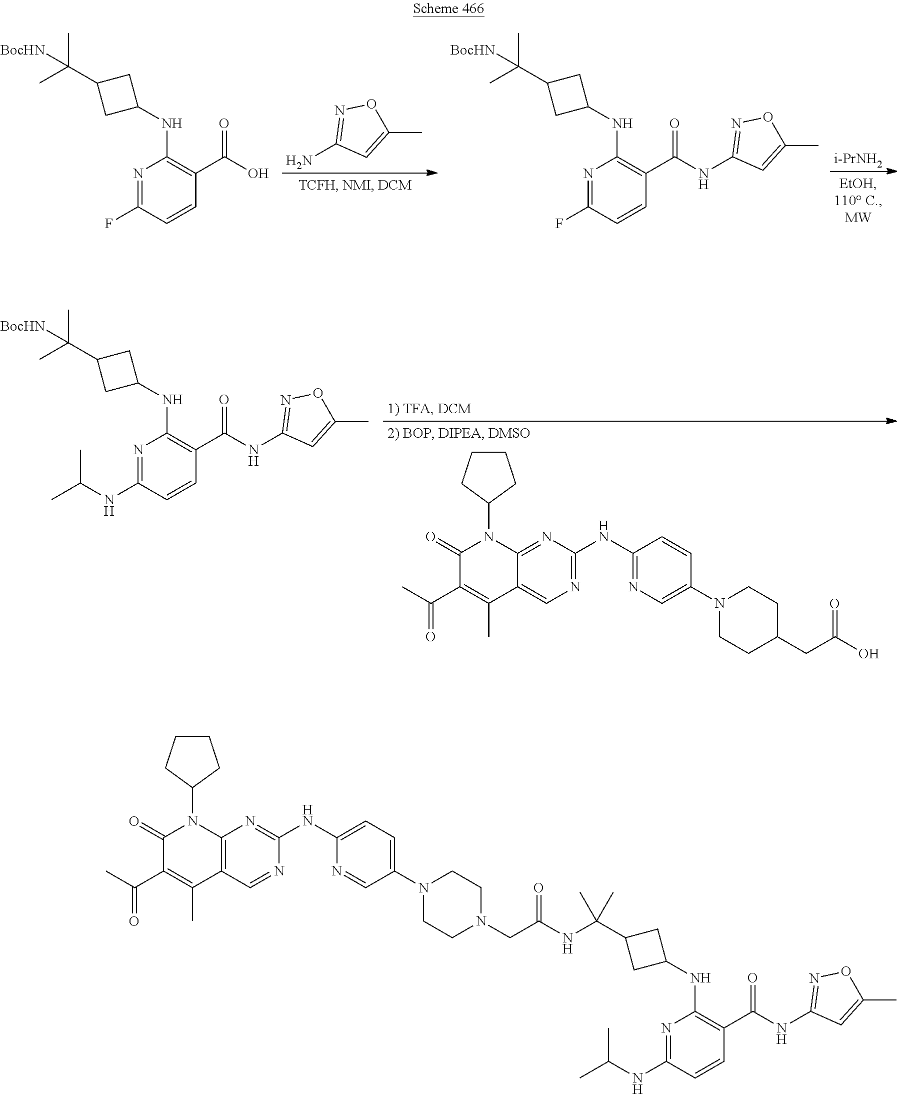

CPD-410 was synthesized following the standard procedures for preparing CPD-144 (10.0 mg, 10% yield over 4 steps) as a yellow solid. MS (ESI) m/z=874.6 [M+H]⁺.

### Example 467. 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(6-(dimethylamino)pyridazin-3-yl)-6-(isopropylamino)nicotinamide (CPD-411)

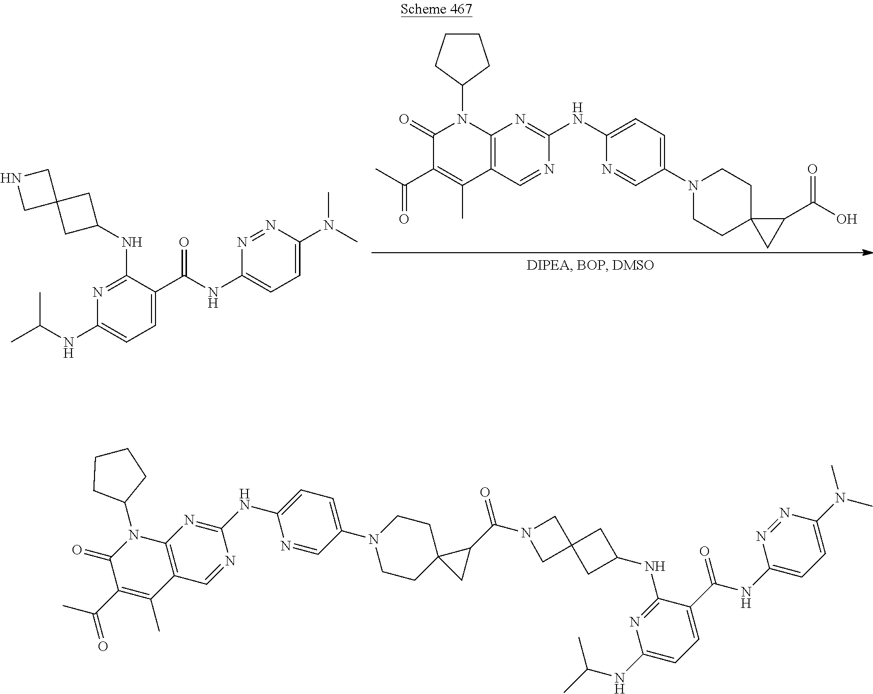 [SMILES: CC(C)Nc1ccc(C(=O)Nc2ccc(N(C)C)nn2)c(NC2CC3(CNC3)C2)n1 ; CC(=O)c1c(C)c2cnc(Nc3ccc(N4CCC5(CC4)CC5C(=O)O)cn3)nc2n(C2CCCC2)c1=O ; CC(=O)c1c(C)c2cnc(Nc3ccc(N4CCC5(CC4)CC5C(=O)N4CC5(CC(Nc6nc(NC(C)C)ccc6C(=O)Nc6ccc(N(C)C)nn6)C5)C4)cn3)nc2n(C2CCCC2)c1=O]

CPD-411 was synthesized following the standard procedure for preparing CPD-261 (10.0 mg, 37% yield) as a yellow solid. MS (ESI) m/z=909.8 [M+H]⁺.

### Example 468. 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(2-cyclopropyl-1-methyl-1H-imidazol-4-yl)-6-(isopropylamino)nicotinamide (CPD-412)

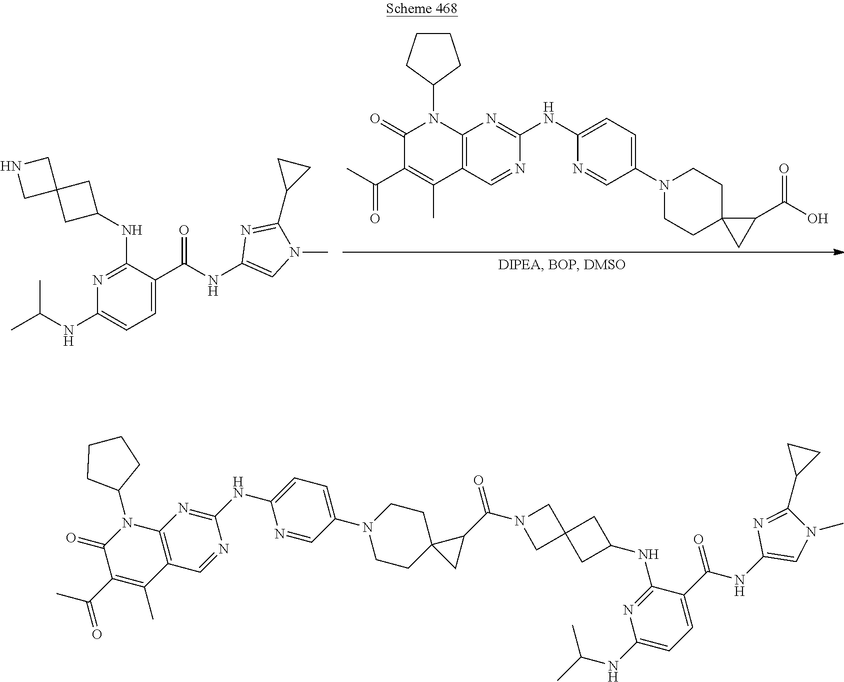

CPD-412 was synthesized following the standard procedure for preparing CPD-261 (9.0 mg, 32% yield) as a yellow solid. MS (ESI) m/z=908.6 [M+H]⁺.

### Example 469. 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(isopropylamino)pyridin-2-yl)amino)cyclobutyl)-6-azaspiro[2.5]octane-1-carboxamide (CPD-413)

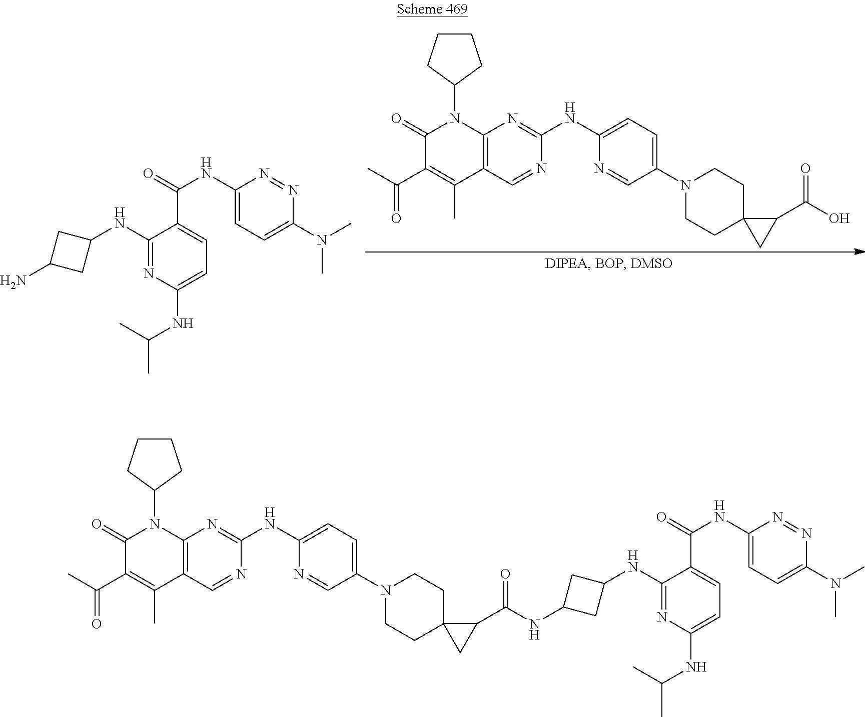

CPD-413 was synthesized following the standard procedure for preparing CPD-261 (8.0 mg, 29% yield) as a yellow solid. MS (ESI) m/z=883.7 [M+H]⁺.

### Example 470. 6-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(isopropylamino)pyridin-2-yl)amino)spiro[3.3]heptan-2-yl)-6-azaspiro[2.5]octane-1-carboxamide (CPD-414)

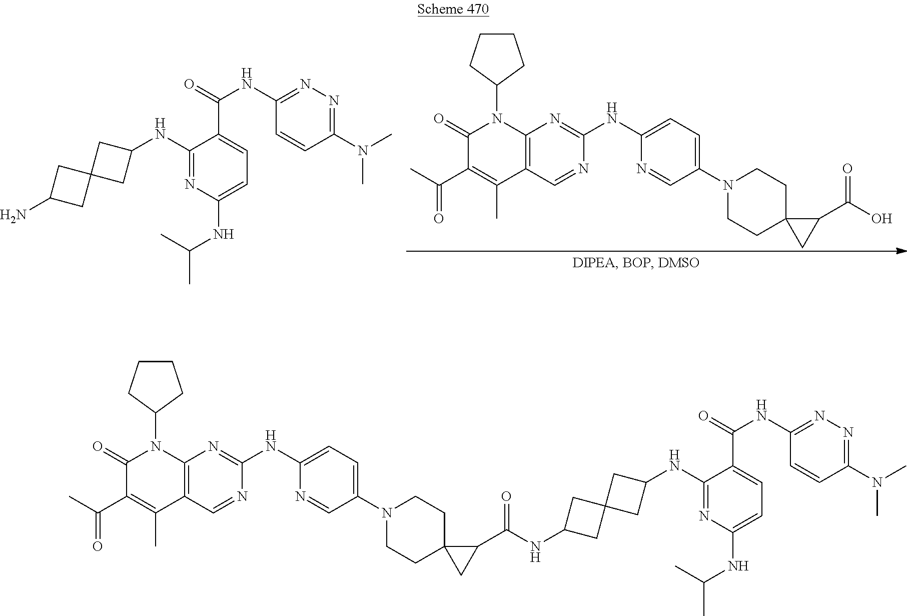

CPD-414 was synthesized following the standard procedure for preparing CPD-261 (10.0 mg, 38% yield) as a yellow solid. MS (ESI) m/z=923.7 [M+H]⁺.

### Example 471. 2-((2-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)-2-azaspiro[3.3]heptan-6-yl)amino)-N-(1,5-dimethyl-1H-pyrazol-3-yl)-6-(isopropylamino)nicotinamide (CPD-415)

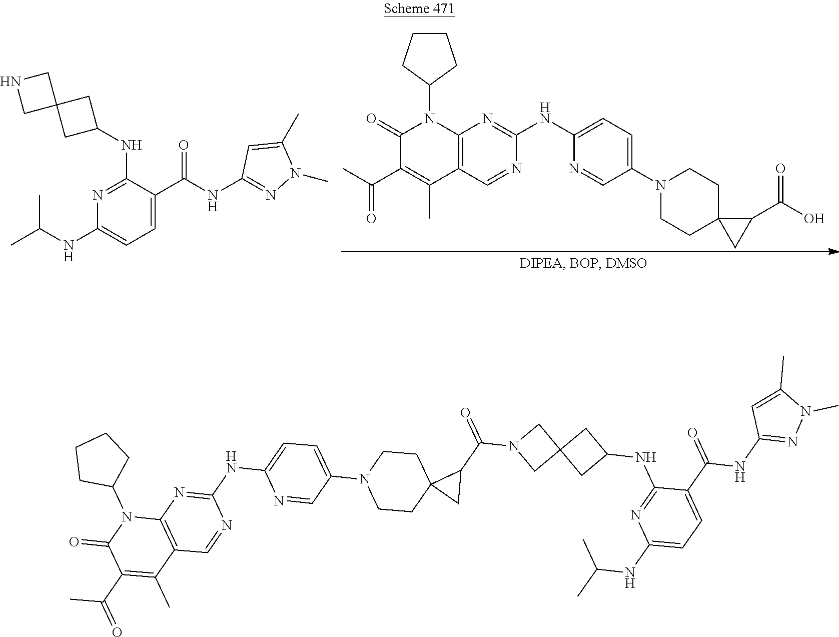

CPD-415 was synthesized following the standard procedure for preparing CPD-261 (8.0 mg, 29% yield) as a yellow solid. MS (ESI) m/z=882.6 [M+H]⁺.

### Example 472. 2-((2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.4]octan-6-yl)amino)-N-(6-(dimethylamino)pyridazin-3-yl)-6-(methylamino)nicotinamide (CPD-416)

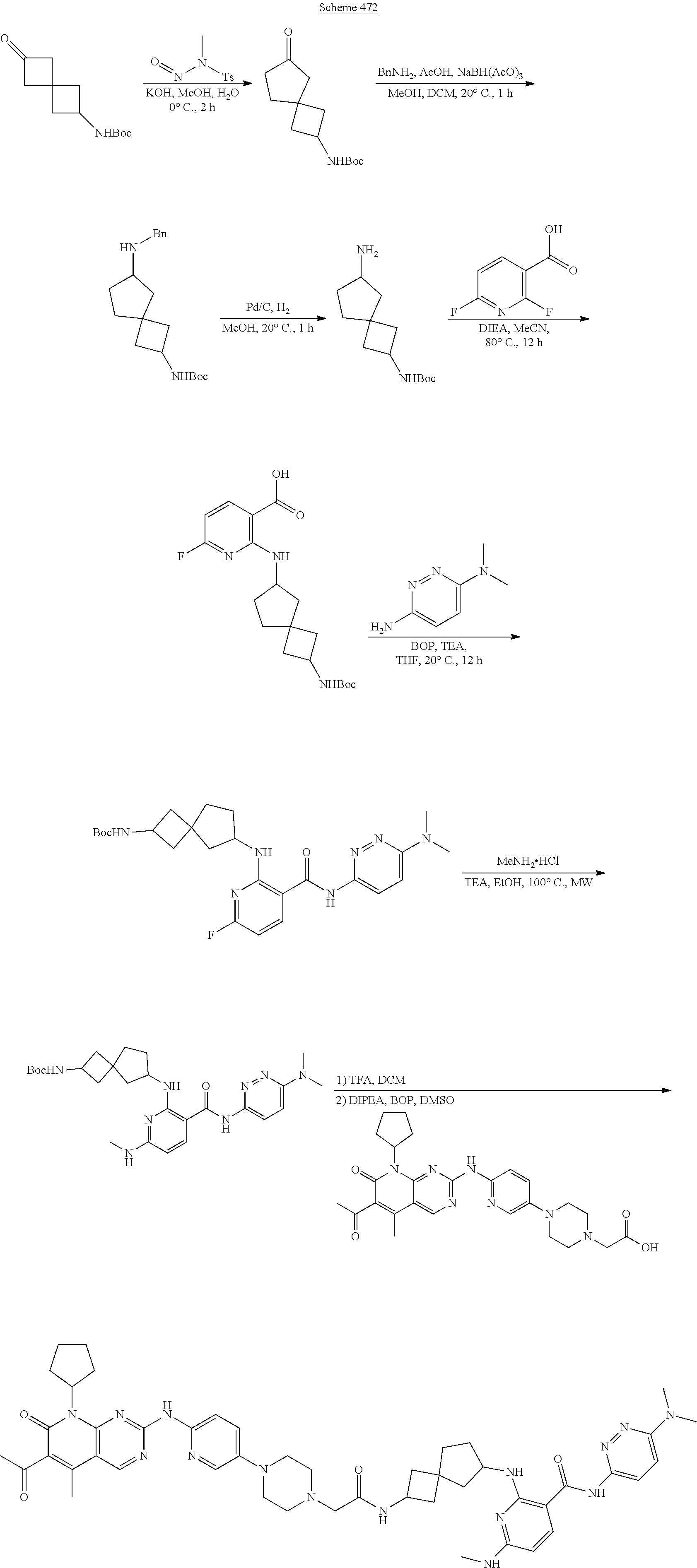

### Step 1. Synthesis of tert-butyl N-(7-oxospiro[3.4]octan-2-yl)carbamate

To a mixture of KOH (3.00 g, 53.5 mmol) in MeOH (10 mL) and H₂O (2 mL) was added tert-butyl N-(2-oxospiro[3.3]heptan-6-yl)carbamate (900 mg, 3.99 mmol) at 0 [symbol] . Then N,4-dimethyl-N-nitroso-benzenesulfonamide (2.16 g, 10.0 mmol) in MeOH (10 mL) was added dropwise. The reaction mixture [symbol] was stirred at 0 for 2 h. The reaction mixture was concentrated in vacuum. The residue was purified by silica gel chromatography (petroleum ether/ethyl acetate=2:1) to provide the desired product (700 mg, 73% yield) as a yellow solid. ¹HNMR (400 Hz, MeOH-d₄) δ 4.08-3.89 (m, 1H), 2.52-2.38 (m, 1H), 2.31-2.27 (m, 1H), 2.26-2.20 (m, 2H), 2.20-2.15 (m, 1H), 2.10-2.03 (m, 11H), 1.97-1.91 (m, 2H), 1.80-1.72 (m, 1H), 1.71-1.61 (m, 1H), 1.43 (s, 9H). MS (ESI) m/z=184.1 [M+H]⁺.

### Step 2. Synthesis of tert-butyl N-[7-(benzylamino)spiro[3.4]octan-2-yl]carbamate

To a solution of tert-butyl N-(7-oxospiro[3.4]octan-2-yl)carbamate (450 mg, 1.88 mmol) in MeOH (6 mL) and dichloromethane (2 mL) were added phenylmethanamine (265 mg, 2.48 mmol, 270 μL), AcOH (524 mg, 8.73 mmol, 0.5 mL) and NaBH(OAc)₃ (900 mg, 4.25 mmol) at 20 [symbol] . The mixture was stirred at 20 [symbol] for 1 h. The reaction mixture was quenched with saturated NaHCO₃ solution and extracted with ethyl acetate (10 mL×3). The combined organic layers were washed with brine (20 mL), dried over Na₂SO₄, filtered, and concentrated to provide the desired product (500 mg, crude) as a yellow oil. MS (ESI) m/z=331.2 [M+H]⁺.

### Step 3. Synthesis of tert-butyl N-(7-aminospiro[3.4]octan-2-yl)carbamate

To a suspension of 10% Pd/C (200 mg) in MeOH (2 mL) was added a solution of tert-butyl N-[7-(benzylamino)spiro[3.4]octan-2-yl]carbamate (500 mg, 1.51 mmol) in MeOH (3 mL) at 20° C. The mixture was stirred at 20° C. for 1 h under H₂ balloon. The reaction mixture was filtered with celite and the filtrate was concentrated to provide the desired product (360 mg, crude) as a yellow oil.

The remaining steps were performed according to the procedures for preparing CPD-353 to provide the desired product (5.0 mg, 2% yield over 5 steps) as a yellow solid. MS (ESI) m/z=898.9 [M+H]⁺.

### Example 473. 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(isopropylamino)pyridin-2-yl)amino)cyclopentyl)-6-azaspiro[2.5]octane-1-carboxamide (CPD-417)

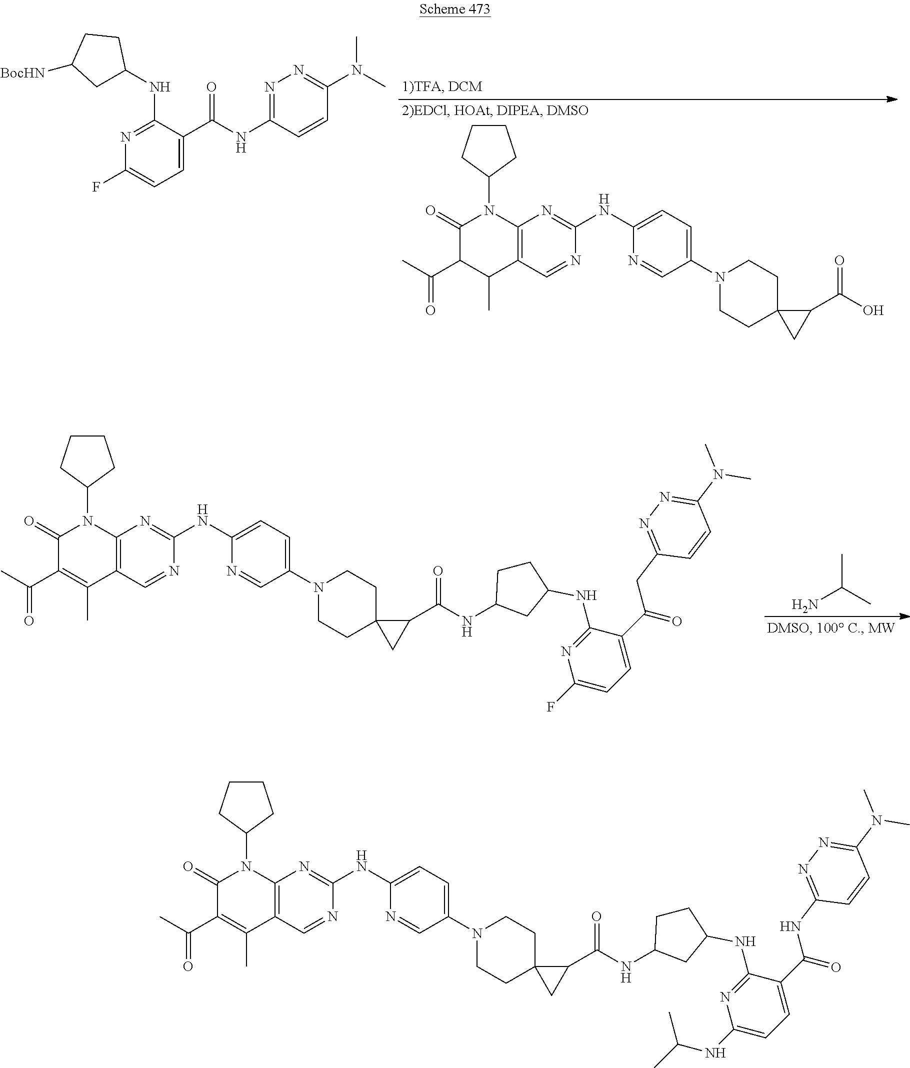

### Step 1. Synthesis of 2-((3-aminocyclopentyl)amino)-N-(6-(dimethylamino)pyridazin-3-yl)-6-fluoronicotinamide

To a solution of tert-butyl (3-((3-((6-(dimethylamino)pyridazin-3-yl)carbamoyl)-6-fluoropyridin-2-yl)amino)cyclopentyl)carbamate (30 mg, 0.06 mmol) in dichloromethane (2 mL) was added TFA (1 mL). The reaction mixture was stirred at rt for 1 h. The mixture was concentrated to give the crude product (30 mg, 970% yield) as a colorless oil. This compound was used directly in the next step without further purification. MS (ESI) mi/z=360.5 [M+H]⁺.

### Step 2. Synthesis of 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-fluoropyridin-2-yl)amino)cyclopentyl)-6-azaspiro[2.5]octane-1-carboxamide

A mixture of 2-((3-aminocyclopentyl)amino)-N-(6-(dimethylamino)pyridazin-3-yl)-6-fluoronicotinamide (30 mg, 0.06 mmol), 6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carboxylic acid (23 mg, 0.44 mmol), EDCI (18 mg, 0.09 mmol), HOAt (13 mg, 0.09 mmol) and DIPEA (41 mg, 0.31 mmol) in DMSO (2 mL) was stirred at rt for 16 h. The reaction mixture was purified by prep-HPLC to give the desired product (16 mg, 29% yield) as a yellow solid. MS (ESI) m/z=858.8 [M+H]⁺.

### Step 3. Synthesis of 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-(isopropylamino)pyridin-2-yl)amino)cyclopentyl)-6-azaspiro[2.5]octane-1-carboxamide

To a solution of 6-(6-((6-acetyl-8-cyclopentyl-5-methyl-7-oxo-7,8-dihydropyrido[2,3-d]pyrimidin-2-yl)amino)pyridin-3-yl)-N-(3-((3-((6-(dimethylamino)pyridazin-3-yl)carbamoyl)-6-fluoropyridin-2-yl)amino)cyclopentyl)-6-azaspiro[2.5]octane-1-carboxamide (16 mg, 0.02 mmol) in DMSO (1 mL) was added propan-2-amine (11 mg, 0.2 mmol). The reaction mixture was stirred at 100° C. under microwave irradiation for 1 h. The reaction mixture was purified by pre-HPLC to give the desired product (3.8 mg, 20% yield) as a yellow solid. MS (ESI) m/z=897.9 [M+H]⁺.

### Example 474. 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)piperidin-4-yl)amino)-N-(6-(dimethylamino)pyridazin-3-yl)-6-(isopropylamino)nicotinamide (CPD-418)

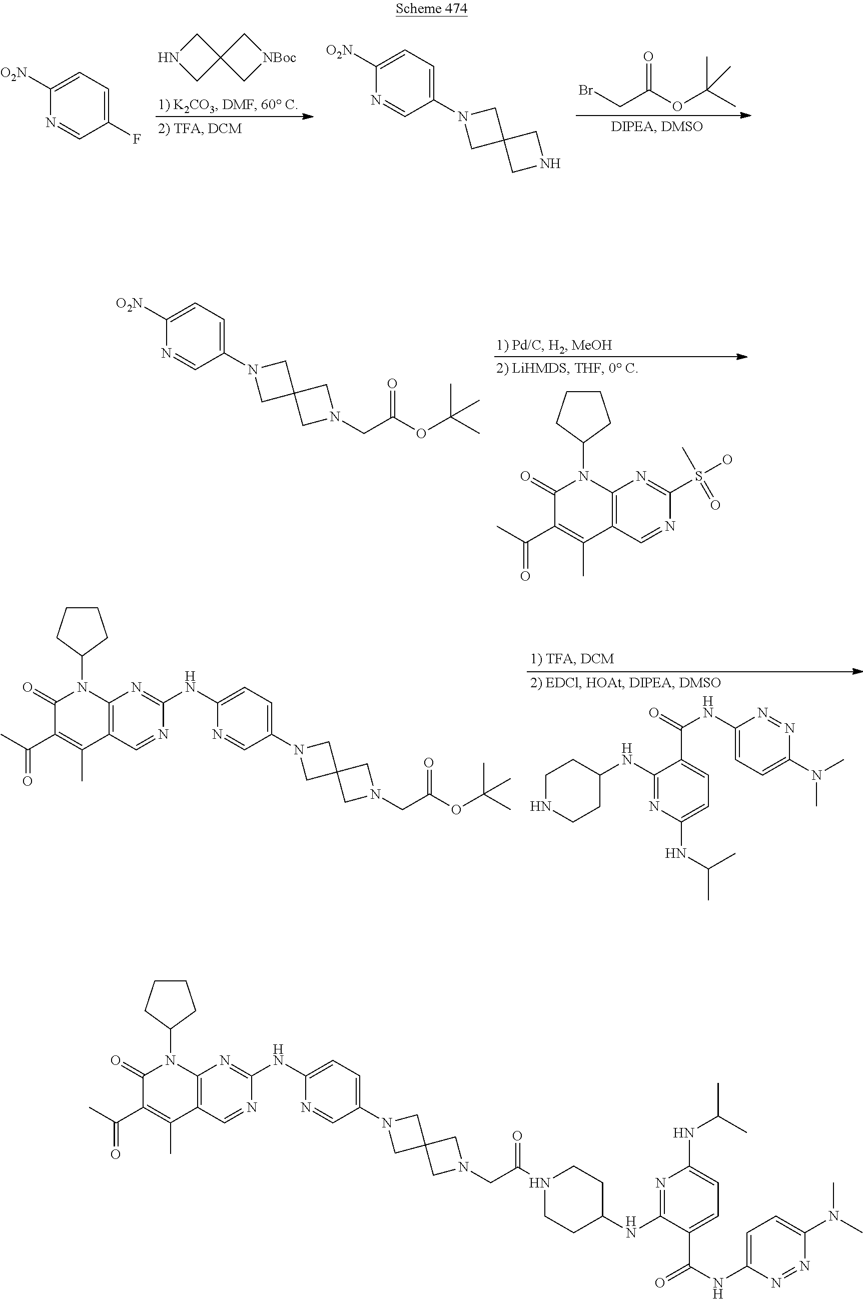

### Step 1. Synthesis of tert-butyl 6-(6-nitropyridin-3-yl)-2,6-diazaspiro[3.3]heptane-2-carboxylate

To a mixture of 5-fluoro-2-nitropyridine (215 mg, 1.5 mmol) and tert-butyl 2,6-diazaspiro[3.3]heptane-2-carboxylate (250 mg, 1.26 mmol) in DMF (10 mL) was added K₂CO₃ (522 mg, 3.7 mmol) at room temperature. The reaction mixture was stirred at 60° C. for 16 h. The reaction mixture was poured into water (20 mL) and extracted with ethyl acetate (3×10 mL). The combined organic layers were washed with saturated brine (20 mL), dried over anhydrous Na₂SO₄, filtered and concentrated. The resulting residue was purified by silica gel flash chromatography to give the desired product (391 mg, 96% yield) as a yellow solid. MS (ESI) m/z=321.6 [M+H]⁺.

### Step 2. Synthesis of 2-(6-nitropyridin-3-yl)-2,6-diazaspiro[3.3]heptane

To a solution of tert-butyl 6-(6-nitropyridin-3-yl)-2,6-diazaspiro[3.3]heptane-2-carboxylate (391 mg, 1.22 mmol) in dichloromethane (2 mL) was added TFA (1 mL). The reaction mixture was stirred at rt for 1 h. The mixture was concentrated to give the crude product (407 mg, 99% yield) as a yellow oil. This compound was used directly in the next step without further purification. MS (ESI) m/z=221.3 [M+H]⁺.

### Step 3. Synthesis of tert-butyl 2-(6-(6-nitropyridin-3-yl)-2,6-diazaspiro[3.3]heptan-2-yl)acetate

To a mixture of 2-(6-nitropyridin-3-yl)-2,6-diazaspiro[3.3]heptane (407 mg, 1.22 mmol) and tert-butyl 2-bromoacetate (285 mg, 1.46 mmol) in DMSO (10 mL) was added DIPEA (472 mg, 3.6 mmol). The reaction mixture was stirred at rt for 16 h. The mixture was poured into water (20 mL) and extracted with ethyl acetate (3×10 mL). The combined organic layers were washed with saturated brine (20 mL), dried over anhydrous Na₂SO₄, filtered and concentrated. The resulting residue was purified by silica gel flash chromatography to give the desired product (310 mg, 76% yield) as a yellow solid. MS (ESI) m/z=335.7 [M+H]⁺.

### Step 4. Synthesis of tert-butyl 2-(6-(6-aminopyridin-3-yl)-2,6-diazaspiro[3.3]heptan-2-yl)acetate

To a solution of tert-butyl-2-(6-(6-nitropyridin-3-yl)-2,6-diazaspiro[3.3]heptan-2-yl)acetate (310 mg, 0.9 mmol) in THF (10 mL) was added 10% Pd/C (20 mg). The reaction mixture was stirred at rt for 16 h under hydrogen balloon. The reaction mixture was filtered and concentrated to give the desired product (220 mg, 77% yield) as a brown oil. MS (ESI) m/z=305.4 [M+H]⁺.

### Step 5. Synthesis of tert-butyl 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetate

To a mixture of tert-butyl 2-(6-(6-aminopyridin-3-yl)-2,6-diazaspiro[3.3]heptan-2-yl)acetate (130 mg, 0.4 mmol) and 6-acetyl-8-cyclopentyl-5-methyl-2-(methylsulfonyl)pyrido[2,3-d]pyrimidin-7(8H)-one (150 mg, 0.4 mmol) in THF (10 mL) was added LiHMDS (0.8 mL, 1 M in THF) at 0° C. The reaction mixture was stirred at 0° C. for 1 h. The reaction mixture was quenched with water (10 mL) and extracted with ethyl acetate (3×10 mL). The combined organic layers were washed with saturated brine (20 mL), dried over anhydrous Na₂SO₄, filtered and concentrated. The resulting residue was purified by silica gel flash chromatography to give the desired product (75 mg, 30% yield) as a yellow oil. MS (ESI) m/z=574.7 [M+H]⁺.

### Step 6. Synthesis of 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetic acid

To a solution of tert-butyl 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetate (75 mg, 0.13 mmol) in dichloromethane (2 mL) was added TFA (1 mL). The reaction mixture was stirred at rt for 1 h. The mixture was concentrated, the resulting residue was purified by reverse-phase chromatography to give the desired product (35 mg, 42% yield) as a brown solid. MS (ESI) m/z=518.7 [M+H]⁺.

### Step 7. Synthesis of 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)piperidin-4-yl)amino)-N-(6-(dimethylamino)pyridazin-3-yl)-6-(isopropylamino)nicotinamide

A mixture of 2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetic acid (10 mg, 0.02 mmol), N-(6-(dimethylamino)pyridazin-3-yl)-6-(isopropylamino)-2-(piperidin-4-ylamino)nicotinamide (7.7 mg, 0.02 mmol), EDCI (5.5 mg, 0.03 mmol), HOAt (3.9 mg, 0.03 mmol) and DIPEA (12 mg, 0.09 mmol) in DMSO (2 mL) was stirred at rt for 16 h. The reaction mixture was purified by reverse-phase chromatography to give the desired product (5.4 mg, 27% yield) as a yellow solid. MS (ESI) m/z=898.9 [M+H]⁺.

### Example 475. 2-((1-(6-(6-((6-acetyl-8-cyclopentyl-5-methyl-7-oxo-7,8-dihydropyrido[2,3-d]pyrimidin-2-yl)amino)pyridin-3-yl)-6-azaspiro[2.5]octane-1-carbonyl)piperidin-4-yl)amino)-6-(isopropylamino)-N-(5-methylisoxazol-3-yl)nicotinamide (CPD-419)

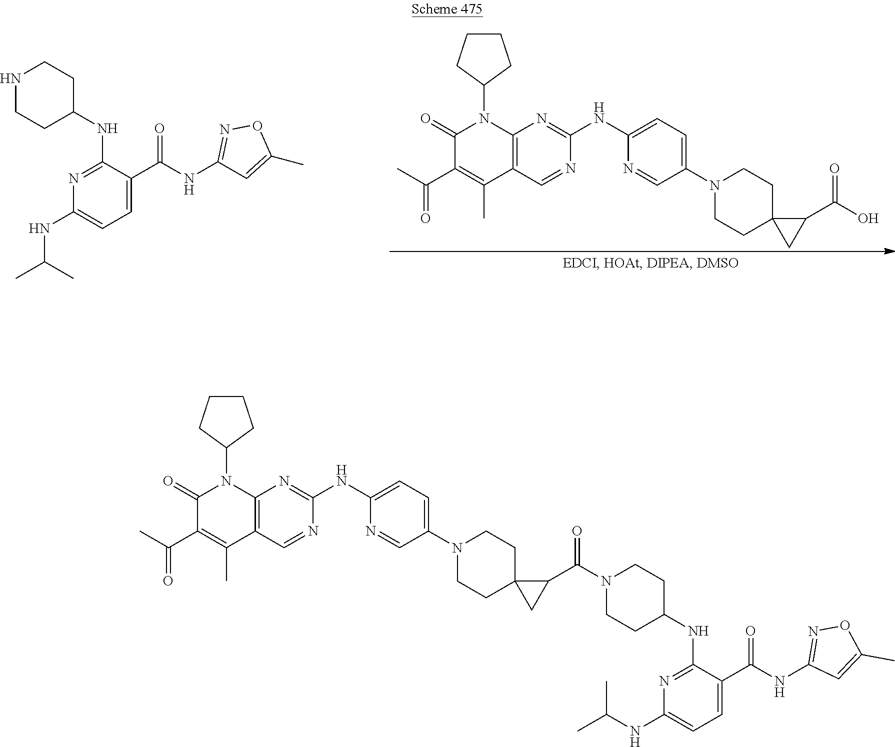 [SMILES: Cc1cc(NC(=O)c2ccc(NC(C)C)nc2NC2CCNCC2)no1 ; CC(=O)c1c(C)c2cnc(Nc3ccc(N4CCC5(CC4)CC5C(=O)O)cn3)nc2n(C2CCCC2)c1=O ; CC(=O)c1c(C)c2cnc(Nc3ccc(N4CCC5(CC4)CC5C(=O)N4CCC(Nc5nc(NC(C)C)ccc5C(=O)Nc5cc(C)on5)CC4)cn3)nc2n(C2CCCC2)c1=O]

CPD-419 was synthesized following the standard procedure for preparing CPD-143 (1.8 mg, 26% yield over 4 steps) as a yellow solid. MS (ESI) m/z=858.0 [M+H]⁺.

### Example 476. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(2-cyclopropyl-1-methyl-1H-imidazol-4-yl)-6-(isopropylamino)nicotinamide (CPD-420)

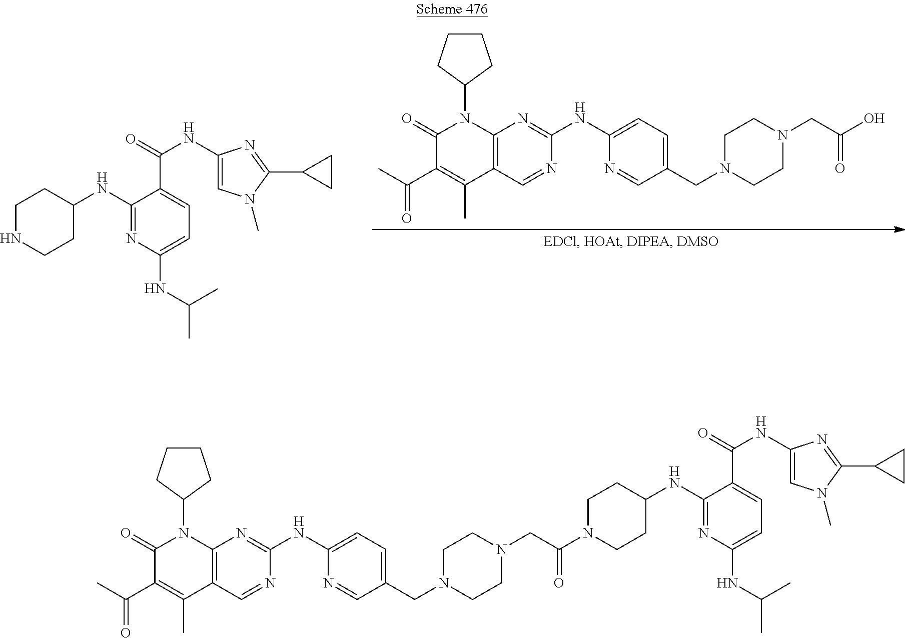 [SMILES: CC(C)Nc1ccc(C(=O)Nc2cn(C)c(C3CC3)n2)c(NC2CCNCC2)n1 ; CC(=O)c1c(C)c2cnc(Nc3ccc(CN4CCN(CC(=O)O)CC4)cn3)nc2n(C2CCCC2)c1=O ; CC(=O)c1c(C)c2cnc(Nc3ccc(CN4CCN(CC(=O)N5CCC(Nc6nc(NC(C)C)ccc6C(=O)Nc6cn(C)c(C7CC7)n6)CC5)CC4)cn3)nc2n(C2CCCC2)c1=O]

CPD-420 was synthesized following the standard procedure for preparing CPD-143 (1.1 mg, 12% yield) as a pale-yellow solid. MS (ESI) m/z=899.9 [M+H]⁺.

### Example 477. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(1,5-dimethyl-1H-pyrazol-3-yl)-6-(isopropylamino)nicotinamide (CPD-421)

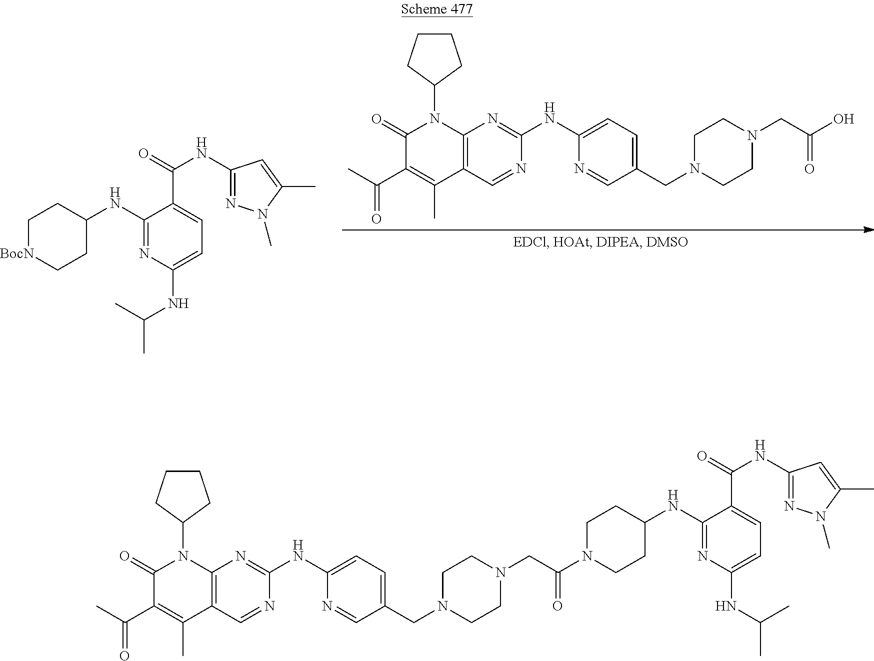

CPD-421 was synthesized following the standard procedure for preparing CPD-143 (1.1 mg, 22% yield) as a yellow solid. MS (ESI) m/z=874.0 [M+H]⁺.

### Example 478. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.3]heptan-6-yl)amino)-N-(6-(dimethylamino)pyridazin-3-yl)-6-(isopropylamino)nicotinamide (CPD-422)

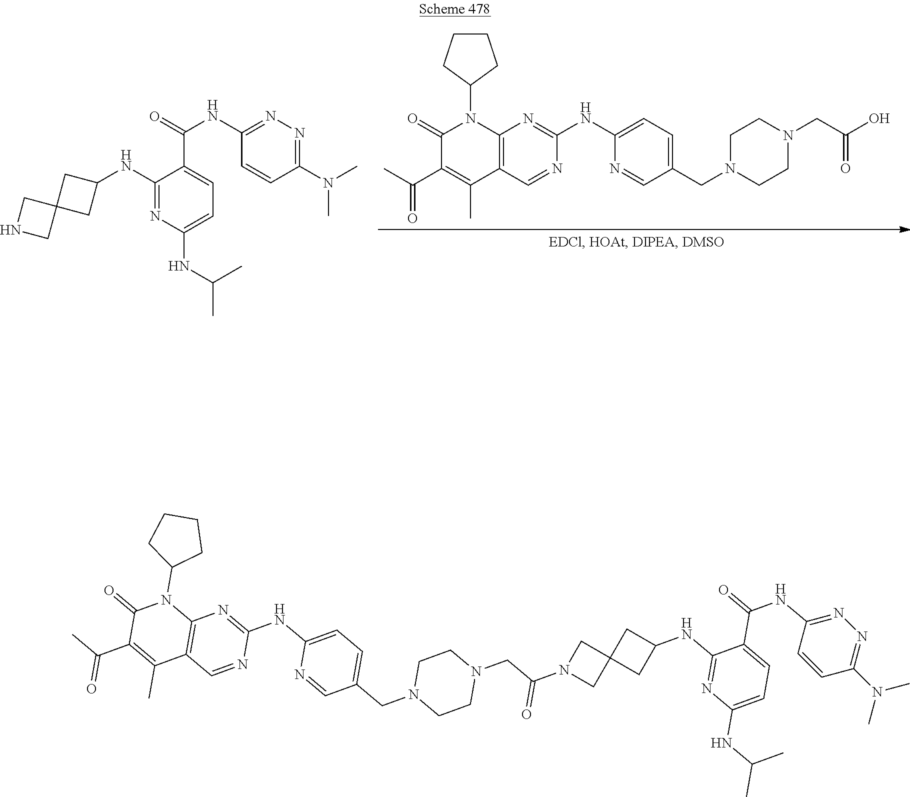

CPD-422 was synthesized following the standard procedure for preparing CPD-143 (1.4 mg, 15% yield) as a yellow solid. MS (ESI) m/z=912.9 [M+H]⁺.

### Example 479. 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)-2-azaspiro[3.3]heptan-6-yl)amino)-N-(6-(dimethylamino)pyridazin-3-yl)-6-(isopropylamino)nicotinamide (CPD-423)

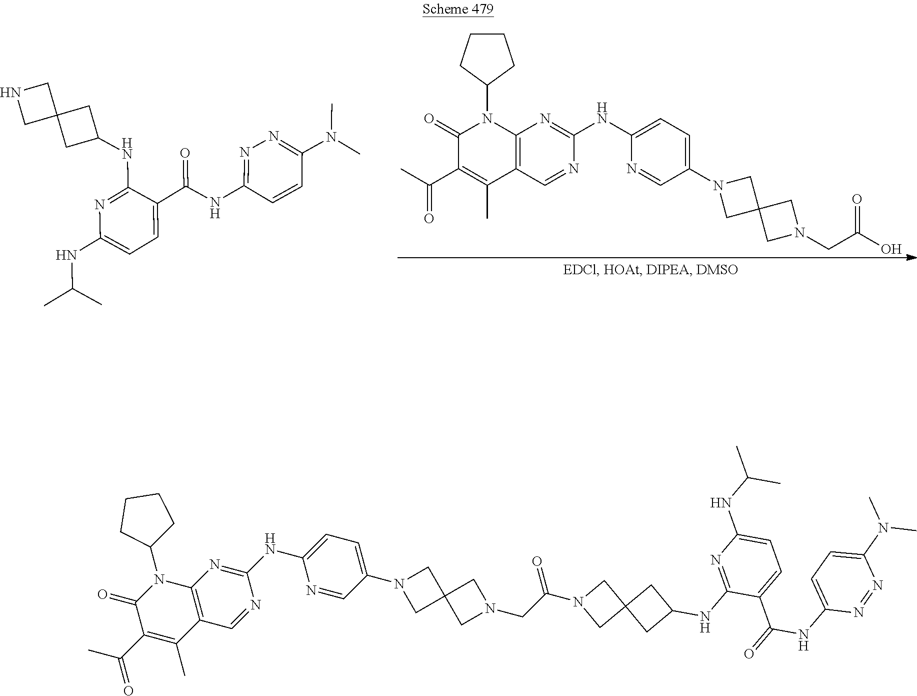

CPD-423 was synthesized following the standard procedure for preparing CPD-143 (2.7 mg, 14% yield) as a yellow solid. MS (ESI) m/z=910.9 [M+H]⁺.

### Example 480. 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)pyrrolidin-3-yl)amino)-N-(6-(dimethylamino)pyridazin-3-yl)-6-(isopropylamino)nicotinamide (CPD-424)

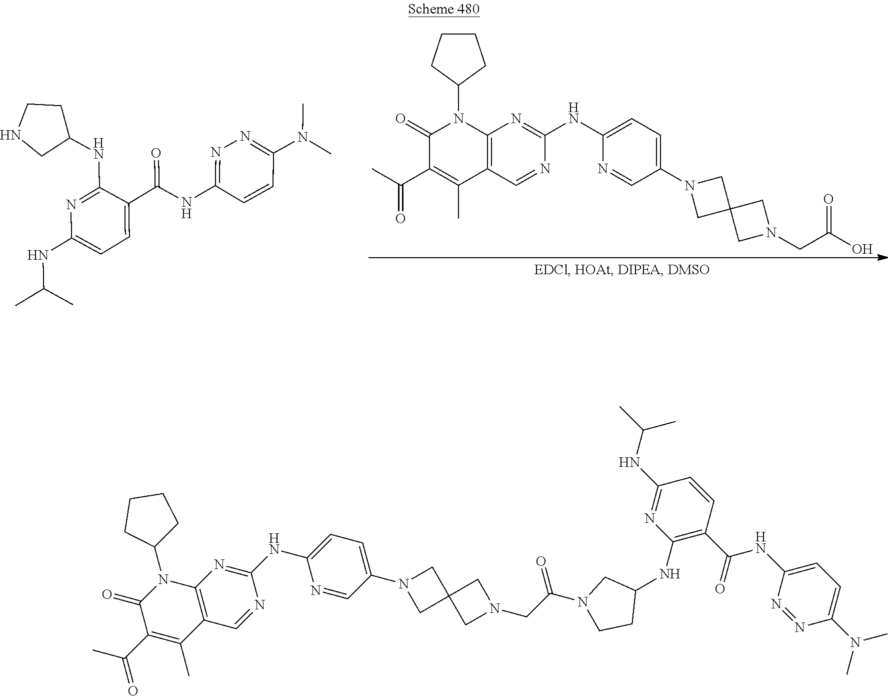

CPD-424 was synthesized following the standard procedure for preparing CPD-143 (3.8 mg, 19% yield) as a yellow solid. MS (ESI) m/z=884.9 [M+H]⁺.

### Example 481. 2-((2-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)-2-azaspiro[3.3]heptan-6-yl)amino)-N-(6-(dimethylamino)pyridazin-3-yl)-6-(isopropylamino)nicotinamide (CPD-425)

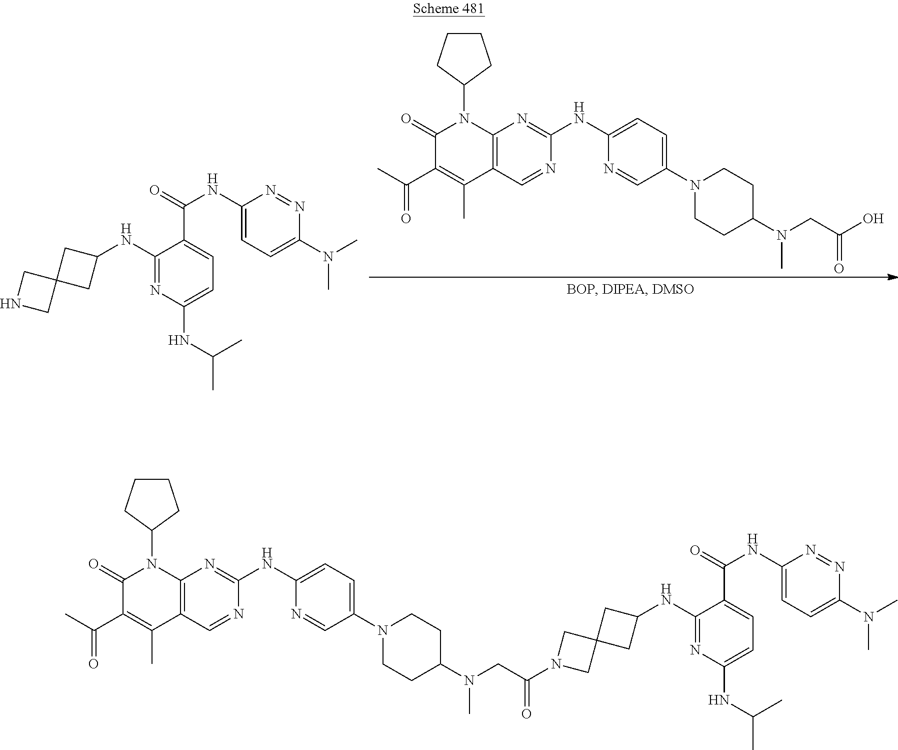 [SMILES: CC(C)Nc1ccc(C(=O)Nc2ccc(N(C)C)nn2)c(NC2CC3(CNC3)C2)n1 ; CC(=O)c1c(C)c2cnc(Nc3ccc(N4CCC(N(C)CC(=O)O)CC4)cn3)nc2n(C2CCCC2)c1=O ; CC(=O)c1c(C)c2cnc(Nc3ccc(N4CCC(N(C)CC(=O)N5CC6(CC(Nc7nc(NC(C)C)ccc7C(=O)Nc7ccc(N(C)C)nn7)C6)C5)CC4)cn3)nc2n(C2CCCC2)c1=O]

CPD-425 was synthesized following the standard procedure for preparing CPD-261 (3.0 mg, 16% yield) as a yellow solid. MS (ESI) m/z=926.7 [M+H]⁺.

### Example 482. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)pyrrolidin-3-yl)amino)-N-(6-(dimethylamino)pyridazin-3-yl)-6-(isopropylamino)nicotinamide (CPD-426)

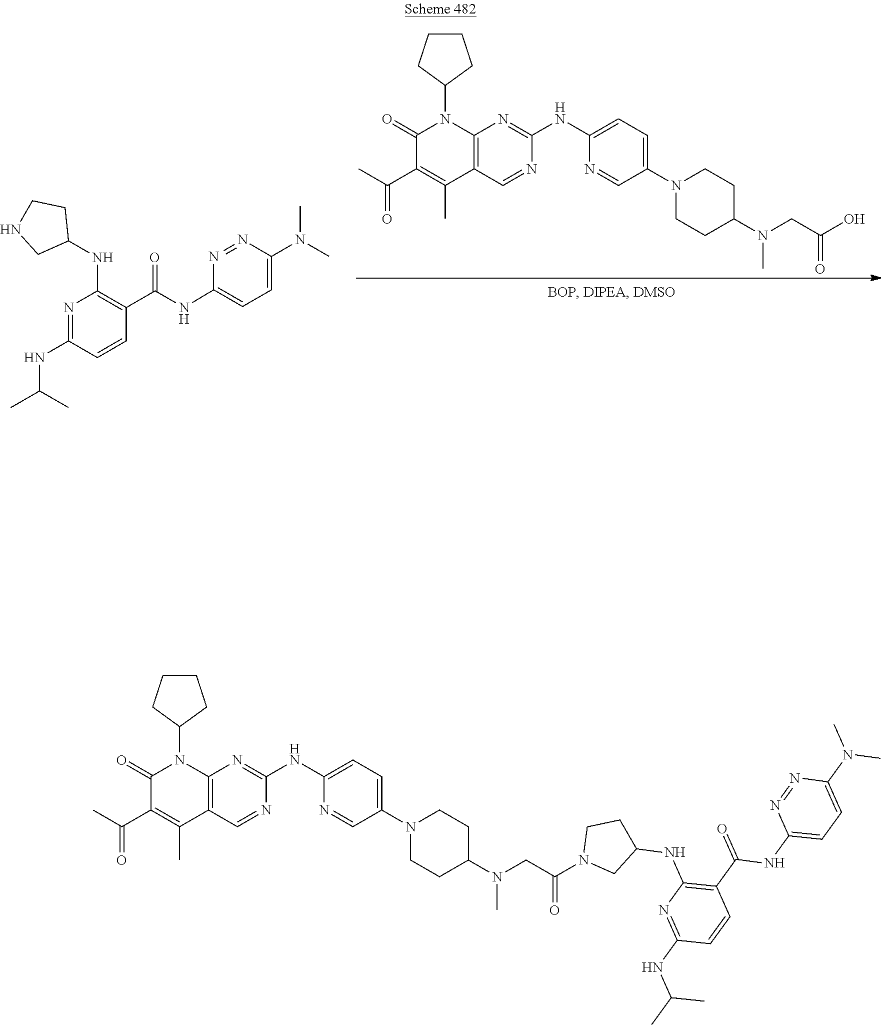

CPD-426 was synthesized following the standard procedure for preparing CPD-261 (5.0 mg, 27% yield) as a yellow solid. MS (ESI) m/z=900.6 [M+H]⁺.

### Example 483. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)azetidin-3-yl)amino)-N-(6-(dimethylamino)pyridazin-3-yl)-6-(isopropylamino)nicotinamide (CPD-427)

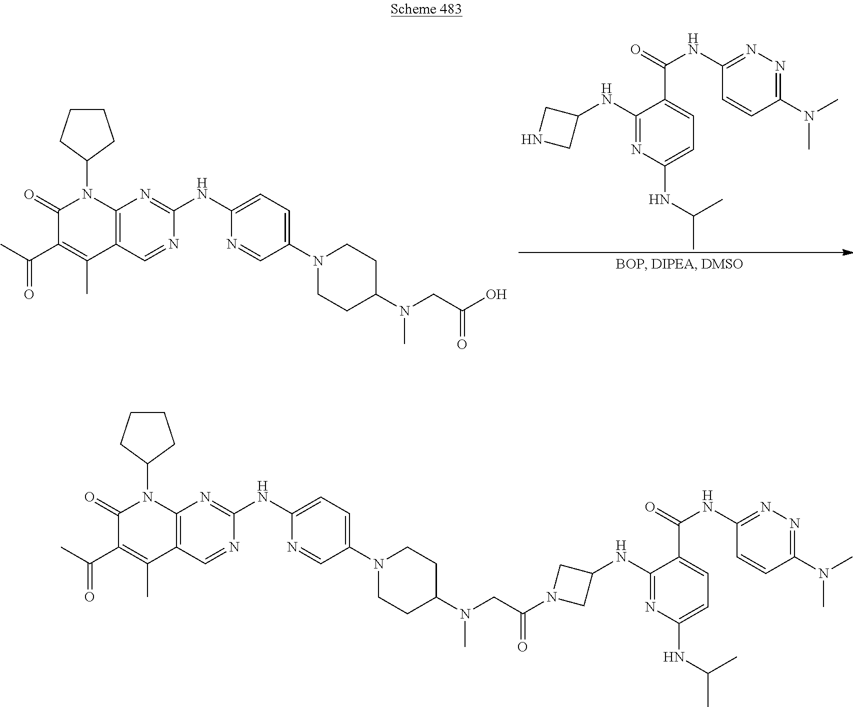 [SMILES: CC(=O)c1c(C)c2cnc(Nc3ccc(N4CCC(N(C)CC(=O)O)CC4)cn3)nc2n(C2CCCC2)c1=O ; CC(C)Nc1ccc(C(=O)Nc2ccc(N(C)C)nn2)c(NC2CNC2)n1 ; CC(=O)c1c(C)c2cnc(Nc3ccc(N4CCC(N(C)CC(=O)N5CC(Nc6nc(NC(C)C)ccc6C(=O)Nc6ccc(N(C)C)nn6)C5)CC4)cn3)nc2n(C2CCCC2)c1=O]

CPD-427 was synthesized following the standard procedure for preparing CPD-261 (4.0 mg, 34% yield) as a yellow solid. MS (ESI) m/z=886.7 [M+H]⁺.

### Example 484. 2-((3-((8-(6-((6-acetyl-8-cyclopentyl-5-methyl-7-oxo-7,8-dihydropyrido[2,3-d]pyrimidin-2-yl)amino)pyridin-3-yl)-8-azaspiro[4.5]decan-2-yl)carbamoyl)cyclobutyl)amino)-N-(6-(dimethylamino)pyridazin-3-yl)-6-(methylamino)nicotinamide (CPD-428)

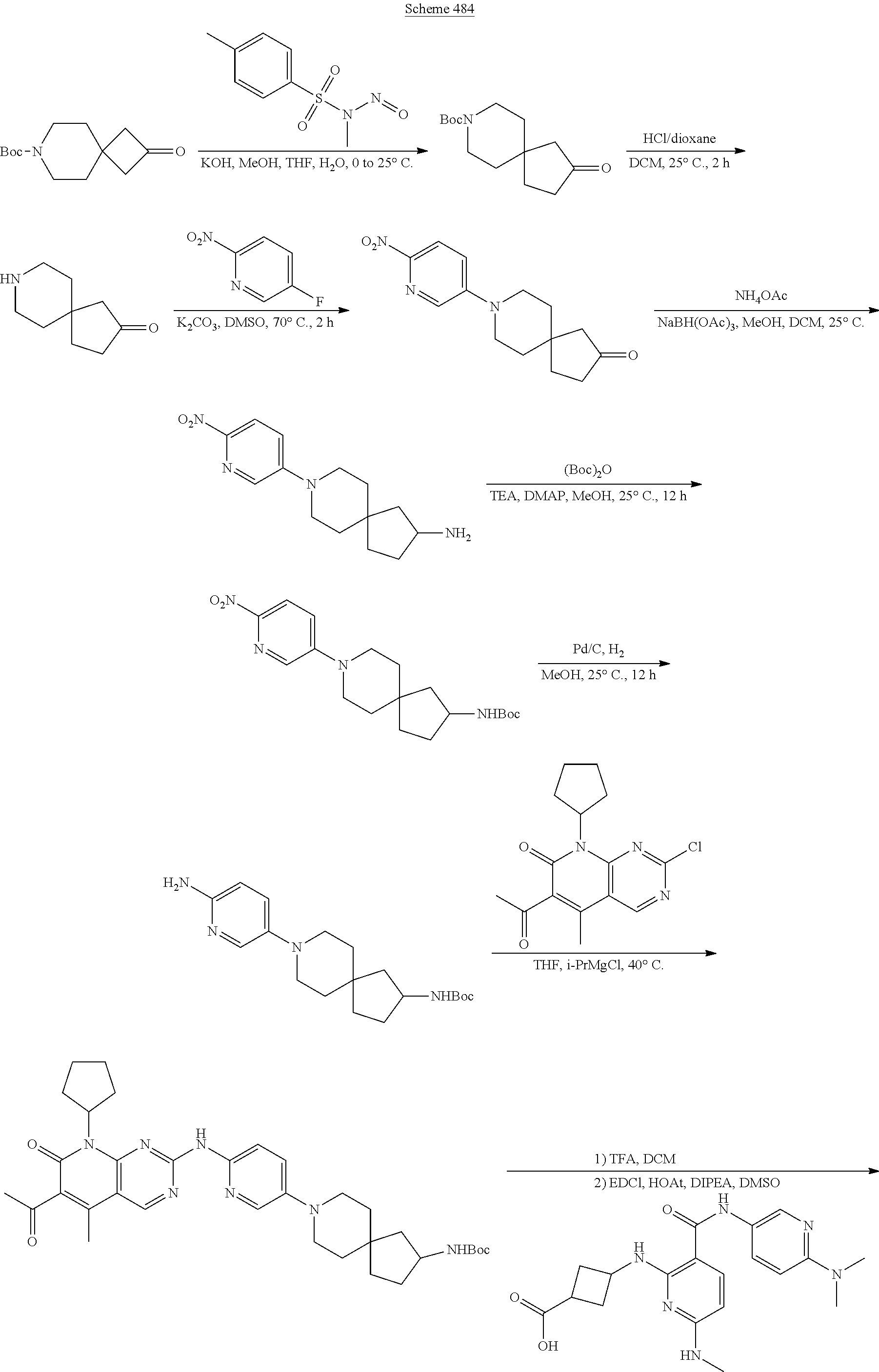

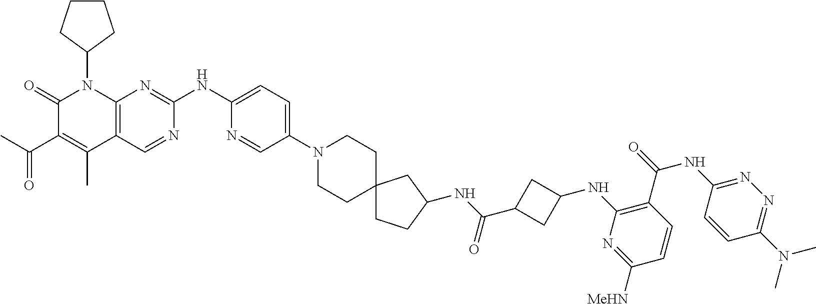 [SMILES: CNc1ccc(C(=O)Nc2ccc(N(C)C)nn2)c(NC2CC(C(=O)NC3CCC4(CCN(c5ccc(Nc6ncc7c(C)c(C(C)=O)c(=O)n(C8CCCC8)c7n6)nc5)CC4)C3)C2)n1]

### Step 1. Synthesis of tert-butyl 3-oxo-8-azaspiro[4.5]decane-8-carboxylate

To a solution of tert-butyl 2-oxo-7-azaspiro[3.5]nonane-7-carboxylate (2.38 g, 9.95 mmol) in MeOH (12 mL) and THF (12 mL) was added KOH (1.43 g, 25.5 mmol) in H₂O (85.5 mL) at 0° C. The N,4-dimethyl-N-nitroso-benzenesulfonamide (5.69 g, 26.6 mmol) in H₂O (85.5 mL) was added dropwise at 0° C. The mixture was stirred at 25° C. for 12 h. The mixture was poured into H₂O (200 mL) and extracted with ethyl acetate (200 mL×3). The combined organic phase was washed with brine (200 mL×3), dried with anhydrous Na₂SO₄, filtered, and concentrated under vacuum. The residue was purified by flash silica gel chromatography (petroleum ether/ethyl acetate=1:1) to provide the desired product (1.83 g, 73% yield) as a colorless oil. ¹HNMR (400 MHz, CDCl₃) δ 3.66-3.54 (m, 2H), 3.25-3.15 (m, 2H), 2.30 (t, J=8.0 Hz, 2H), 2.16 (s, 2H), 1.87 (t, J=8.0 Hz, 1H), 1.56-1.50 (m, 4H), 1.47-1.45 (m, 9H)

### Step 2. Synthesis of 8-azaspiro[4.5]decan-3-one

To a solution of tert-butyl 3-oxo-8-azaspiro[4.5]decane-8-carboxylate (1.83 g, 7.22 mmol) in dichloromethane (20 mL) was added HCl solution (4 M in dioxane, 18.3 mL). The mixture was stirred at 25° C. for 2 h. The reaction mixture was concentrated under vacuum to provide the desired product (1.60 g, crude) as a white oil. ¹HNMR (400 MHz, CDCl₃) δ 9.66 (s, 2H), 3.32-3.30 (m, 2H), 3.13-3.11 (s, 2H), 2.36 (t, J=8.0 Hz, 2H), 2.23 (s, 2H), 2.02-1.94 (m, 4H), 1.92-1.86 (m, 2H) The remaining steps were performed according to the procedures for preparing CPD-388 to provide the desired product (3.1 mg, 2% yield over 7 steps) as a light-yellow solid. MS (ESI) m/z=884.0 [M+H]⁺.

### Example 485. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)methyl)bicyclo[1.1.1]pentan-1-yl)amino)-N-(6-(dimethylamino)pyridazin-3-yl)-6-(isopropylamino)nicotinamide (CPD-429)

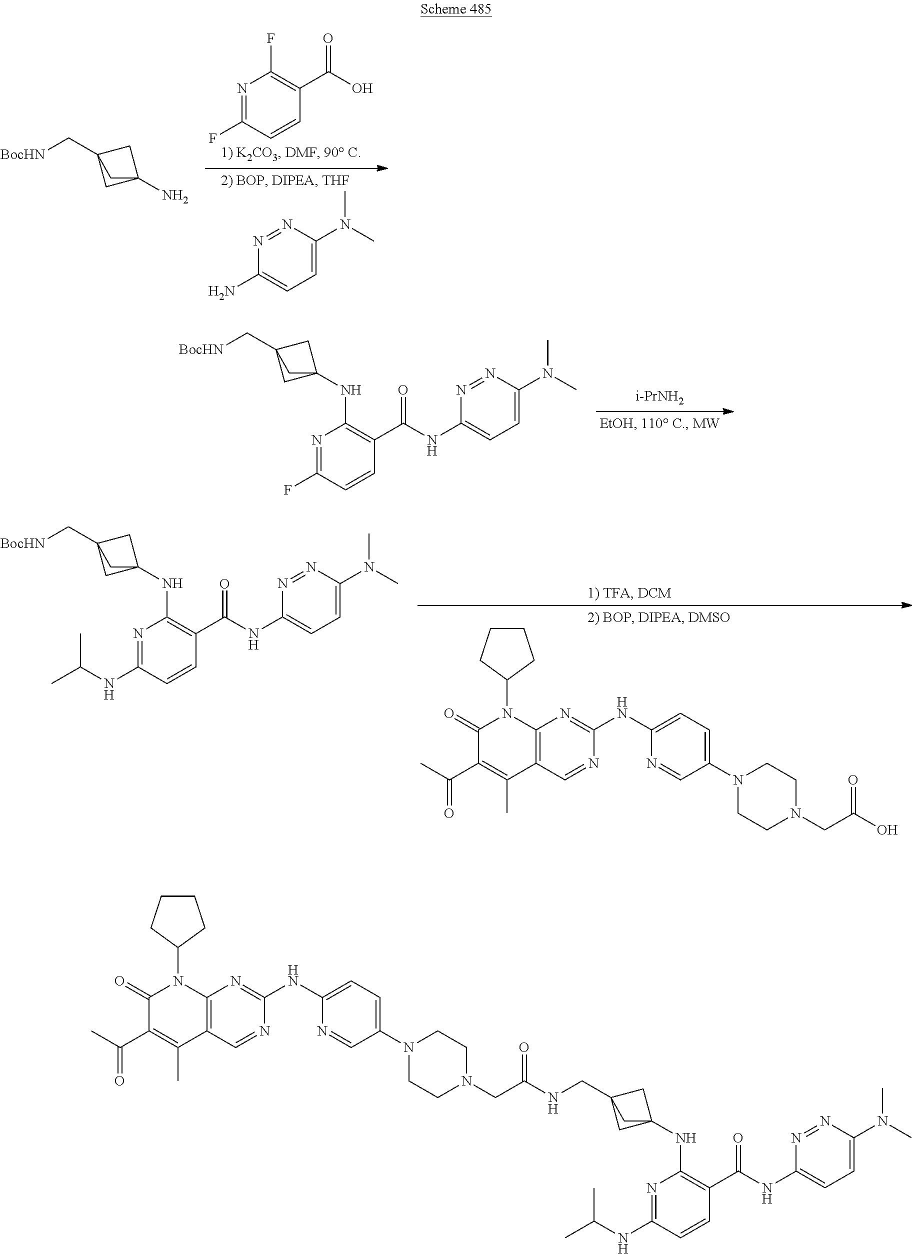

CPD-429 was synthesized following the standard procedures for preparing CPD-145 (6.0 mg, 5% yield over 5 steps) as a yellow solid. MS (ESI) m/z 898.7 [M+H]⁺.

### Example 486. 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-N-(6-(dimethylamino)pyridazin-3-yl)-6-(methylamino)nicotinamide (CPD-430)

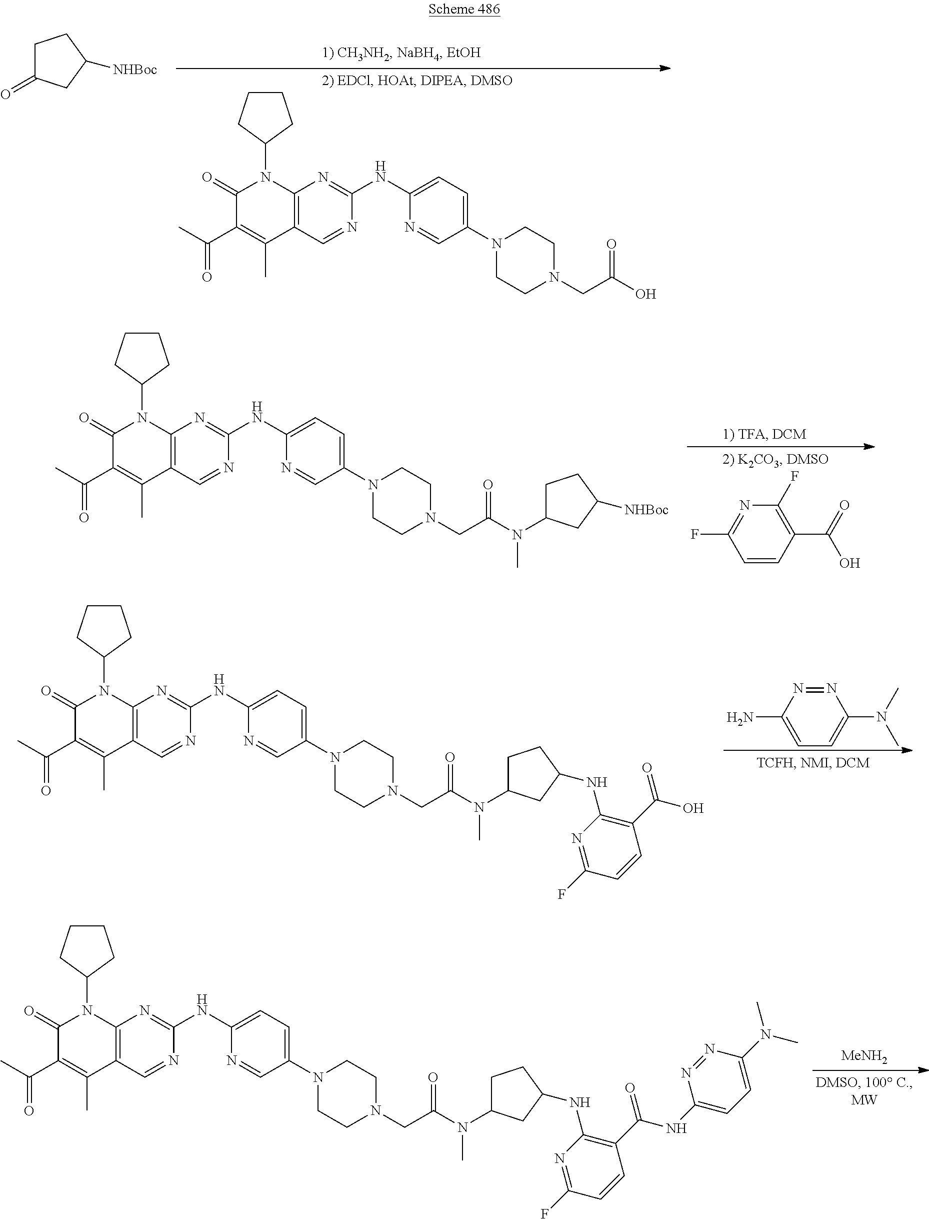

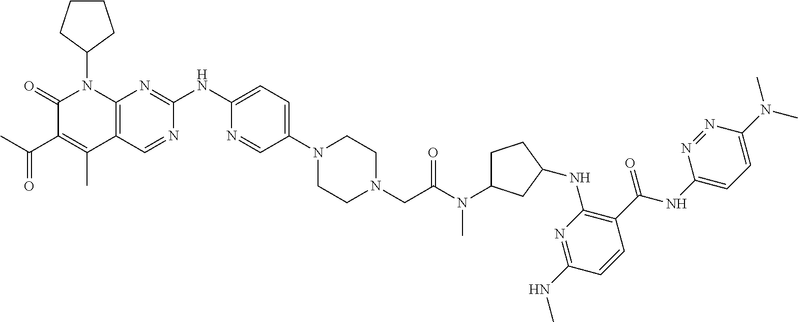 [SMILES: CNc1ccc(C(=O)Nc2ccc(N(C)C)nn2)c(NC2CCC(N(C)C(=O)CN3CCN(c4ccc(Nc5ncc6c(C)c(C(C)=O)c(=O)n(C7CCCC7)c6n5)nc4)CC3)C2)n1]

### Step 1. Synthesis of tert-butyl (3-(methylamino)cyclopentyl)carbamate

To a solution of tert-butyl (3-oxocyclopentyl)carbamate (216 mg, 1.09 mmol) in EtOH (5 mL) was added methylamine ethanol solution (224 mg, 2.17 mmol). The reaction mixture was stirred at rt for 16 h. NaBH₄ (82 mg, 2.17 mmol) was added at 0° C. The reaction mixture was stirred at rt for 1 h. The mixture was concentrated and purified by silica gel chromatography to give the desired product (150 mg, 64% yield) as a white solid. MS (ESI) m/z=215.6 [M+H]⁺.

### Step 2. Synthesis of tert-butyl (3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)carbamate

A mixture of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (100 mg, 0.2 mmol), tert-butyl (3-(methylamino)cyclopentyl)carbamate (42 mg, 0.2 mmol), EDCI (56 mg, 0.3 mmol), HOAt (40 mg, 0.3 mmol) and DIPEA (127 mg, 0.9 nmol) in DMSO (2 mL) was stirred at rt for 16 h. The reaction mixture was purified by reverse-phase chromatography to give the desired product (55 mg, 39% yield) as a yellow solid. MS (ESI) m/z=702.9 [M+H]⁺.

### Step 3. Synthesis of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-(3-aminocyclopentyl)-N-methylacetamide

To a solution of tert-butyl (3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)carbamate (55 mg, 0.07 mmol) in dichloromethane (2 mL) was added TFA (1 mL). The reaction mixture was stirred at rt for 1 h. The mixture was concentrated to give the desired product (55 mg, 98% yield) as a brown oil. This compound was used directly in the next step without further purification. MS (ESI) m/z=602.8 [M+H]⁺.

### Step 4. Synthesis of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-6-fluoronicotinic acid

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-(3-aminocyclopentyl)-N-methylacetamide (55 mg, 0.09 mmol) in DMSO (2 mL) were added 2,6-difluoronicotinic acid (17 mg, 0.1 mmol) and K₂CO₃ (38 mg, 0.2 mmol). The reaction mixture was stirred 90° C. for 3 h. The reaction mixture was purified by reverse-phase chromatography to give the desired product (45 mg, 57% yield) as a yellow solid. MS (ESI) m/z=741.9 [M+H]⁺.

### Step 5. Synthesis of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-N-(6-(dimethylamino)pyridazin-3-yl)-6-fluoronicotinamide

A mixture of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-6-fluoronicotinic acid (15 mg, 0.02 mmol), N,N-dimethylpyridazine-3,6-diamine (2.8 mg, 0.02 mmol), TCFH (11 mg, 0.04 mmol) and NMI (8.3 mg, 0.1 mmol) in dichloromethane (5 mL) was stirred at rt for 1 h. The reaction mixture was concentrated and purified by reverse-phase chromatography to give the desired product (8.5 mg, 43% yield) as a colorless oil. MS (ESI) m/z=861.8 [M+H]⁺.

### Step 6. Synthesis of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-N-(6-(dimethylamino)pyridazin-3-yl)-6-(methylamino)nicotinamide

To a solution of 2-((3-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)cyclopentyl)amino)-N-(6-(dimethylamino)pyridazin-3-yl)-6-fluoronicotinamide (8.5 mg, 0.01 mmol) in DMSO (1 mL) was added MeNH₂ solution (30% in EtOH, 1 mL). The reaction mixture was stirred at 100° C. for 1 h under microwave irradiation. The reaction mixture was purified by reverse-phase chromatography to give the desired product (1.2 mg, 12% yield) as a yellow solid. MS (ESI) m/z=873.0 [M+H]⁺.

### Example 487. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanoyl)piperidin-4-yl)amino)-N-(2-cyclopropyl-1-methyl-1H-imidazol-4-yl)-6-(isopropylamino)nicotinamide (CPD-431)

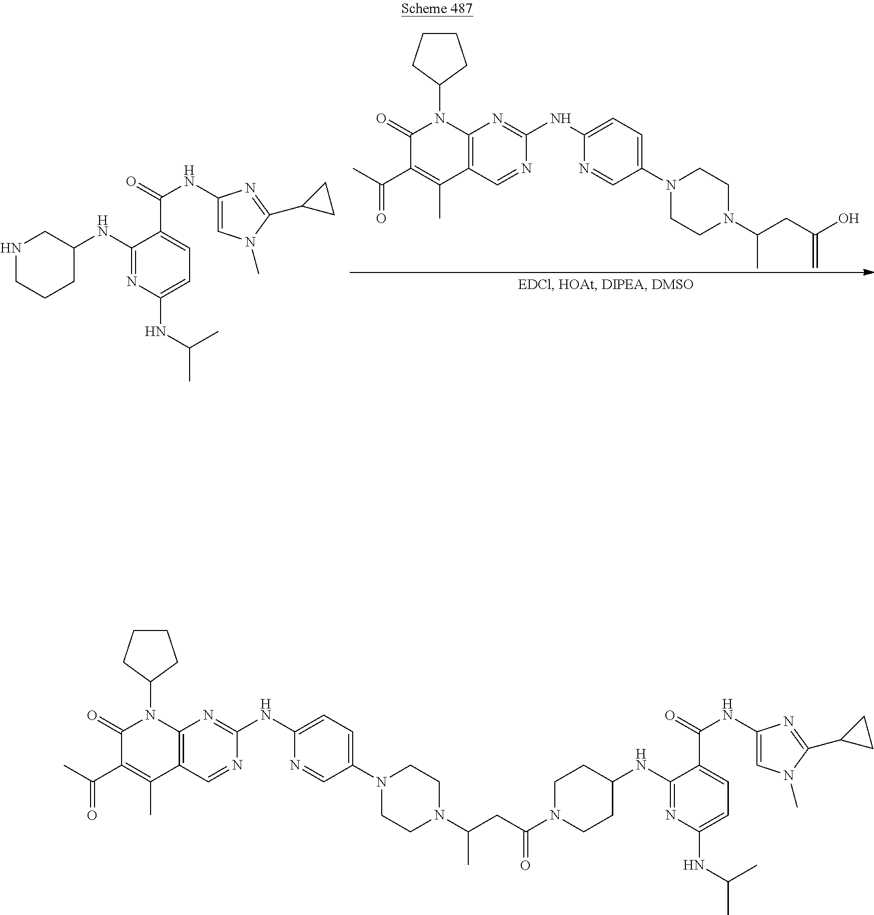

CPD-431 was synthesized following the standard procedure for preparing CPD-143 (2.8 mg, 30% yield) as a yellow solid. MS (ESI) m/z=913.8 [M+H]⁺.

### Example 488. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanoyl)-2-azaspiro[3.3]heptan-6-yl)amino)-N-(6-(dimethylamino)pyridazin-3-yl)-6-((methyl-d₃)amino)nicotinamide (CPD-432)

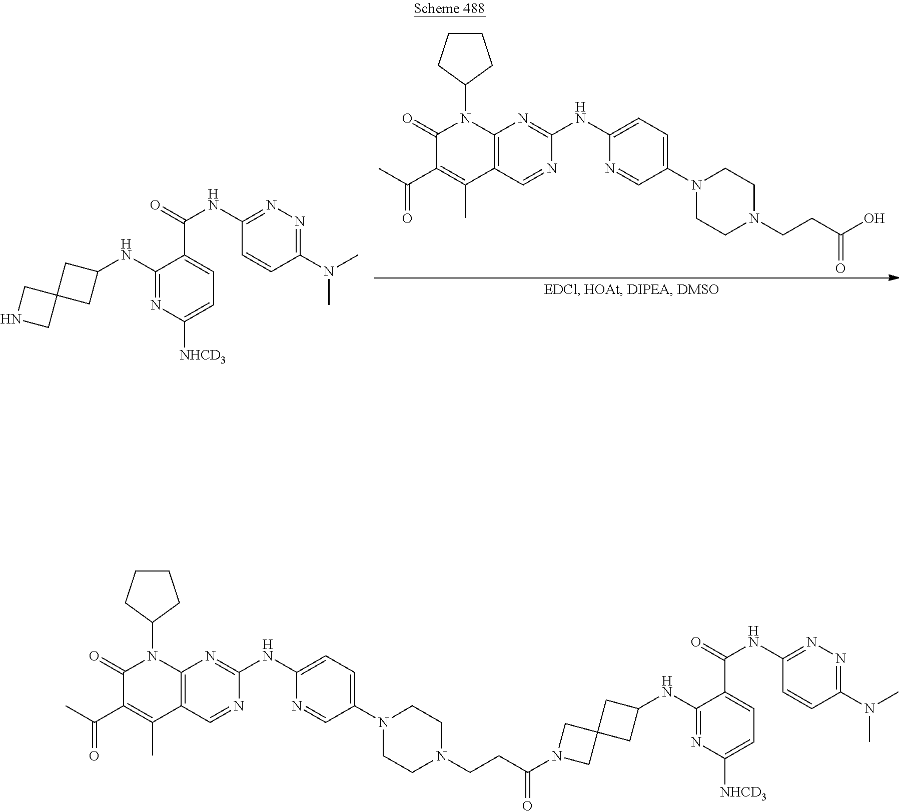

CPD-432 was synthesized following the standard procedure for preparing CPD-143 (2.1 mg, 25% yield) as a yellow solid. MS (ESI) m/z=887.7 [M+H]⁺.

### Example 489. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(1,5-dimethyl-1H-pyrazol-3-yl)-6-(isopropylamino)nicotinamide (CPD-433)

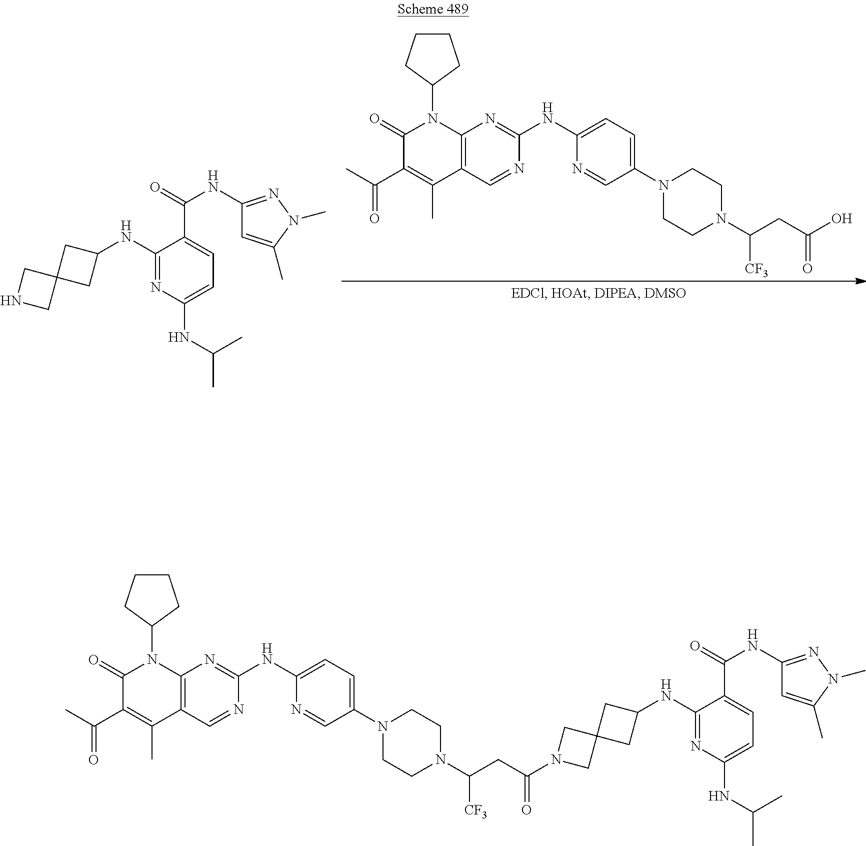 [SMILES: Cc1cc(NC(=O)c2ccc(NC(C)C)nc2NC2CC3(CNC3)C2)nn1C ; CC(=O)c1c(C)c2cnc(Nc3ccc(N4CCN(C(CC(=O)O)C(F)(F)F)CC4)cn3)nc2n(C2CCCC2)c1=O ; CC(=O)c1c(C)c2cnc(Nc3ccc(N4CCN(C(CC(=O)N5CC6(CC(Nc7nc(NC(C)C)ccc7C(=O)Nc7cc(C)n(C)n7)C6)C5)C(F)(F)F)CC4)cn3)nc2n(C2CCCC2)c1=O]

CPD-433 was synthesized following the standard procedure for preparing CPD-143 (2.3 mg, 28% yield) as a yellow solid. MS (ESI) m/z=953.8 [M+H]⁺.

### Example 490. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(1,5-dimethyl-1H-pyrazol-3-yl)-6-((methyl-d3)amino)nicotinamide (CPD-434)

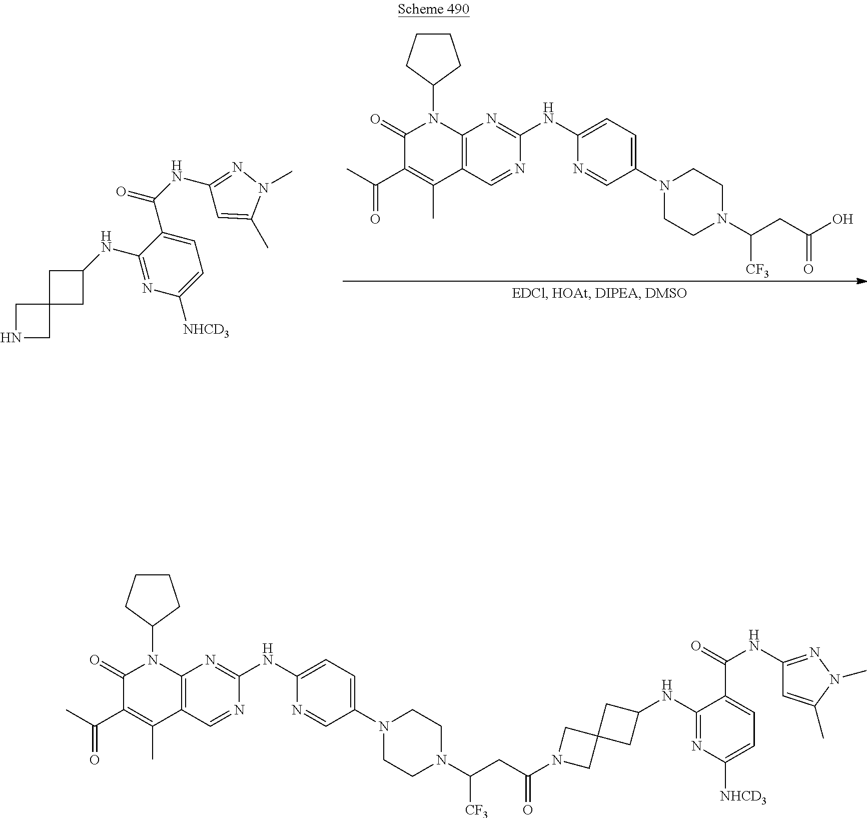

CPD-434 was synthesized following the standard procedure for preparing CPD-143 (2.1 mg, 27% yield) as a yellow solid. MS (ESI) m/z=928.8 [M+H]⁺.

### Example 491. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(2-cyclopropyl-1-methyl-1H-imidazol-4-yl)-6-((methyl-d₃)amino)nicotinamide (CPD-435)

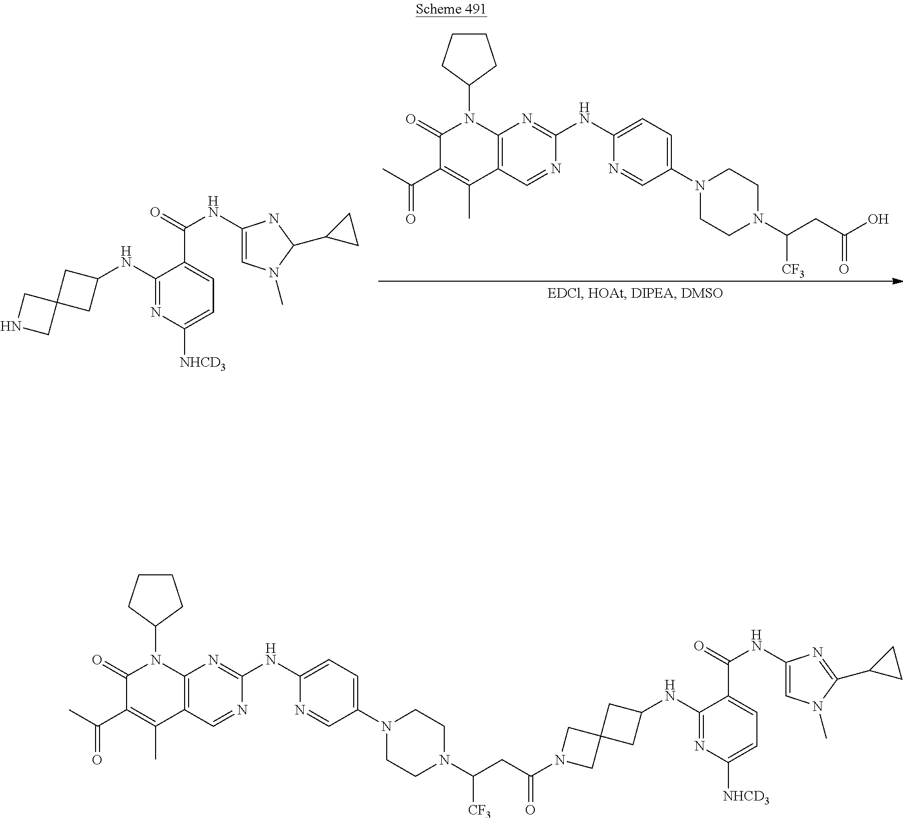

CPD-435 was synthesized following the standard procedure for preparing CPD-143 (2.1 mg, 25% yield) as a yellow solid. MS (ESI) m/z=954.8 [M+H]⁺.

### Example 492. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(2-cyclopropyl-1-methyl-1H-imidazol-4-yl)-6-(isopropylamino)nicotinamide (CPD-436)

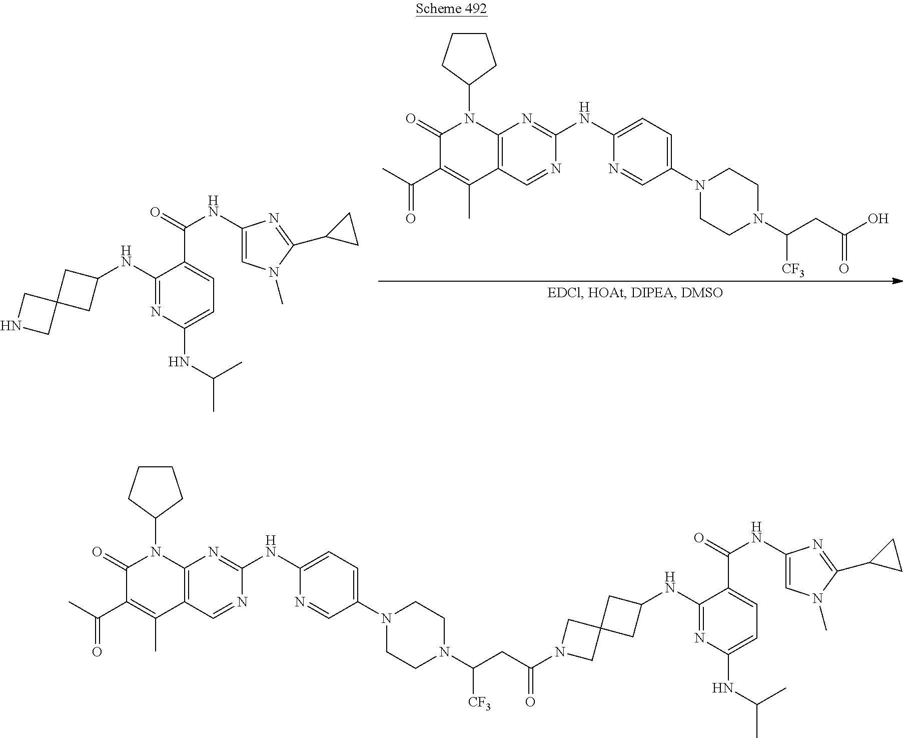 [SMILES: CC(C)Nc1ccc(C(=O)Nc2cn(C)c(C3CC3)n2)c(NC2CC3(CNC3)C2)n1 ; CC(=O)c1c(C)c2cnc(Nc3ccc(N4CCN(C(CC(=O)O)C(F)(F)F)CC4)cn3)nc2n(C2CCCC2)c1=O ; CC(=O)c1c(C)c2cnc(Nc3ccc(N4CCN(C(CC(=O)N5CC6(CC(Nc7nc(NC(C)C)ccc7C(=O)Nc7cn(C)c(C8CC8)n7)C6)C5)C(F)(F)F)CC4)cn3)nc2n(C2CCCC2)c1=O]

CPD-436 was synthesized following the standard procedure for preparing CPD-143 (2.1 mg, 26% yield) as a yellow solid. MS (ESI) m/z=979.9 [M+H]⁺.

### Example 493. 7-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-(isopropylamino)pyridin-2-yl)amino)spiro[3.3]heptan-2-yl)-7-azaspiro[3.5]nonane-2-carboxamide (CPD-437)

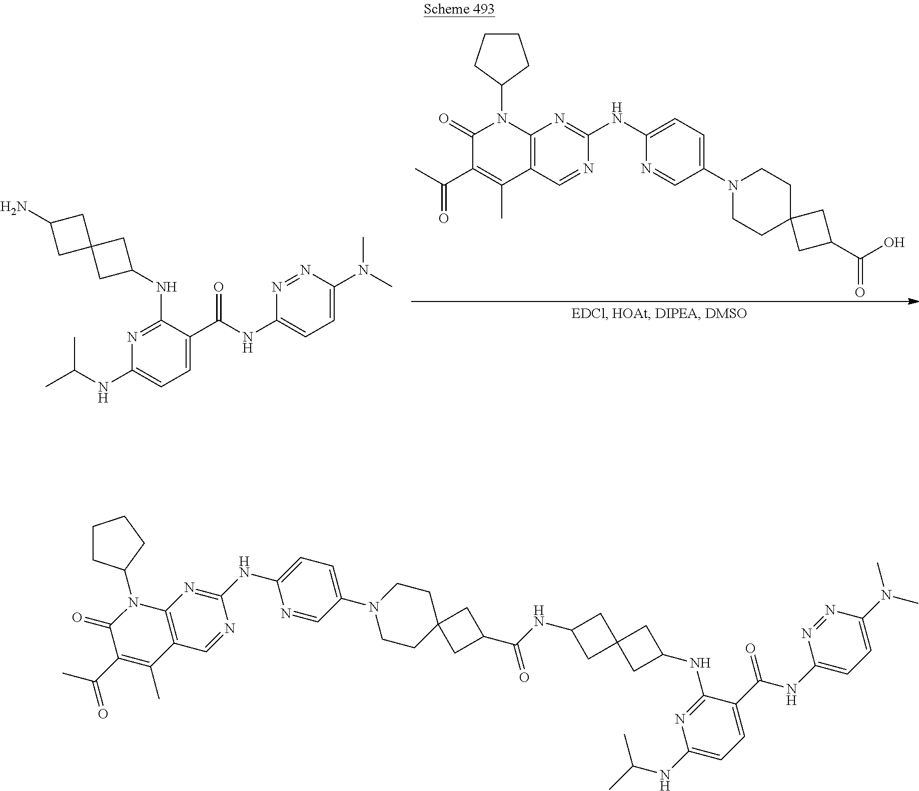 [SMILES: CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CCC5(CC4)CC(C(=O)O)C5)cn3)nc2n(C2CCCC2)c1=O ; CC(=O)c1c(C)c2cnc(Nc3ccc(N4CCC5(CC4)CC(C(=O)NC4CC6(C4)CC(Nc4nc(NC(C)C)ccc4C(=O)Nc4ccc(N(C)C)nn4)C6)C5)cn3)nc2n(C2CCCC2)c1=O]

CPD-437 was synthesized following the standard procedure for preparing CPD-143 (1.3 mg, 11% yield) as a yellow solid. MS (ESI) m/z=938.1 [M+H]⁺.

### Example 494. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)spiro[3.3]heptan-2-yl)amino)-N-(6-(dimethylamino)pyridazin-3-yl)-6-((methyl-d₃)amino)nicotinamide (CPD-438)

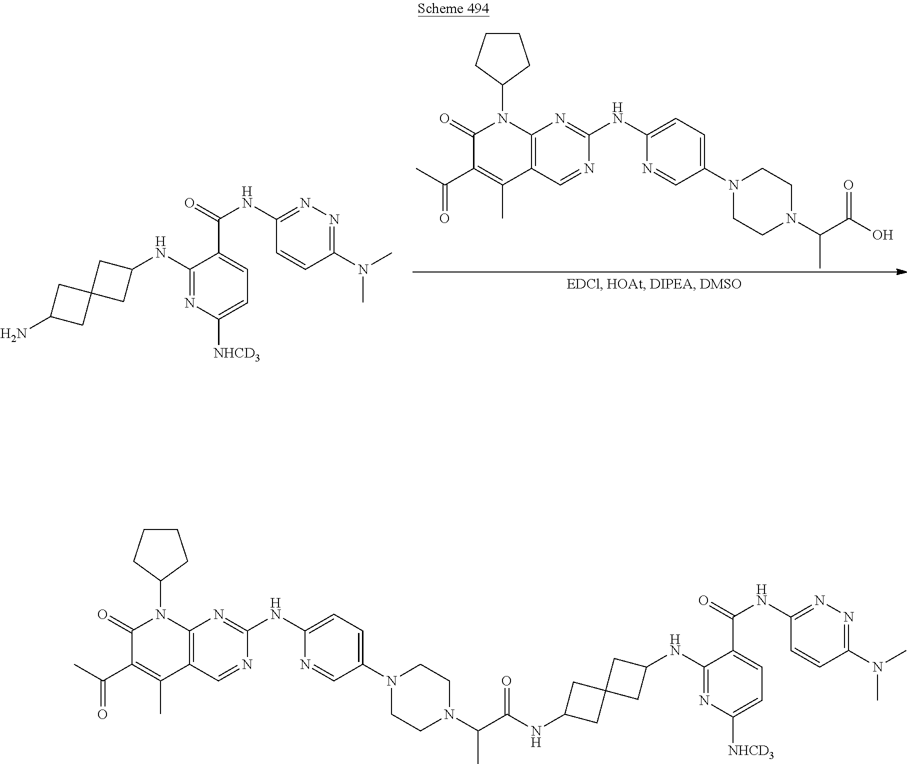

CPD-438 was synthesized following the standard procedure for preparing CPD-143 (1.5 mg, 12% yield) as a yellow solid. MS (ESI) m/z=902.0 [M+H]⁺.

### Example 495. 6-(6-((6-acetyl-8-cyclopentyl-5-methyl-7-oxo-7,8-dihydropyrido[2,3-d]pyrimidin-2-yl)amino)pyridin-3-yl)-N-(6-((3-((6-(dimethylamino)pyridazin-3-yl)carbamoyl)-6-((methyl-d₃)amino)pyridin-2-yl)amino)spiro[3.3]heptan-2-yl)-6-azaspiro[2.5]octane-1-carboxamide (CPD-439)

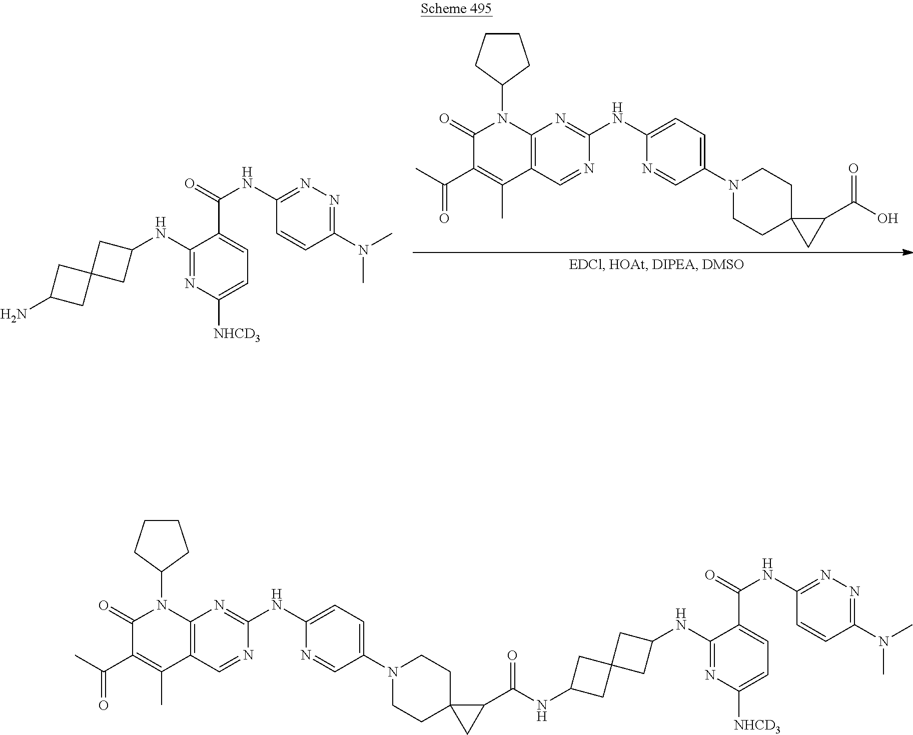

CPD-439 was synthesized following the standard procedure for preparing CPD-143 (2.1 mg, 16% yield) as a yellow solid. MS (ESI) m/z=899.0 [M+H]⁺.

### Example 496. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(5,6-dihydro-4H-pyrrolo[1,2-b]pyrazol-2-yl)-6-(isopropylamino)nicotinamide (CPD-440)

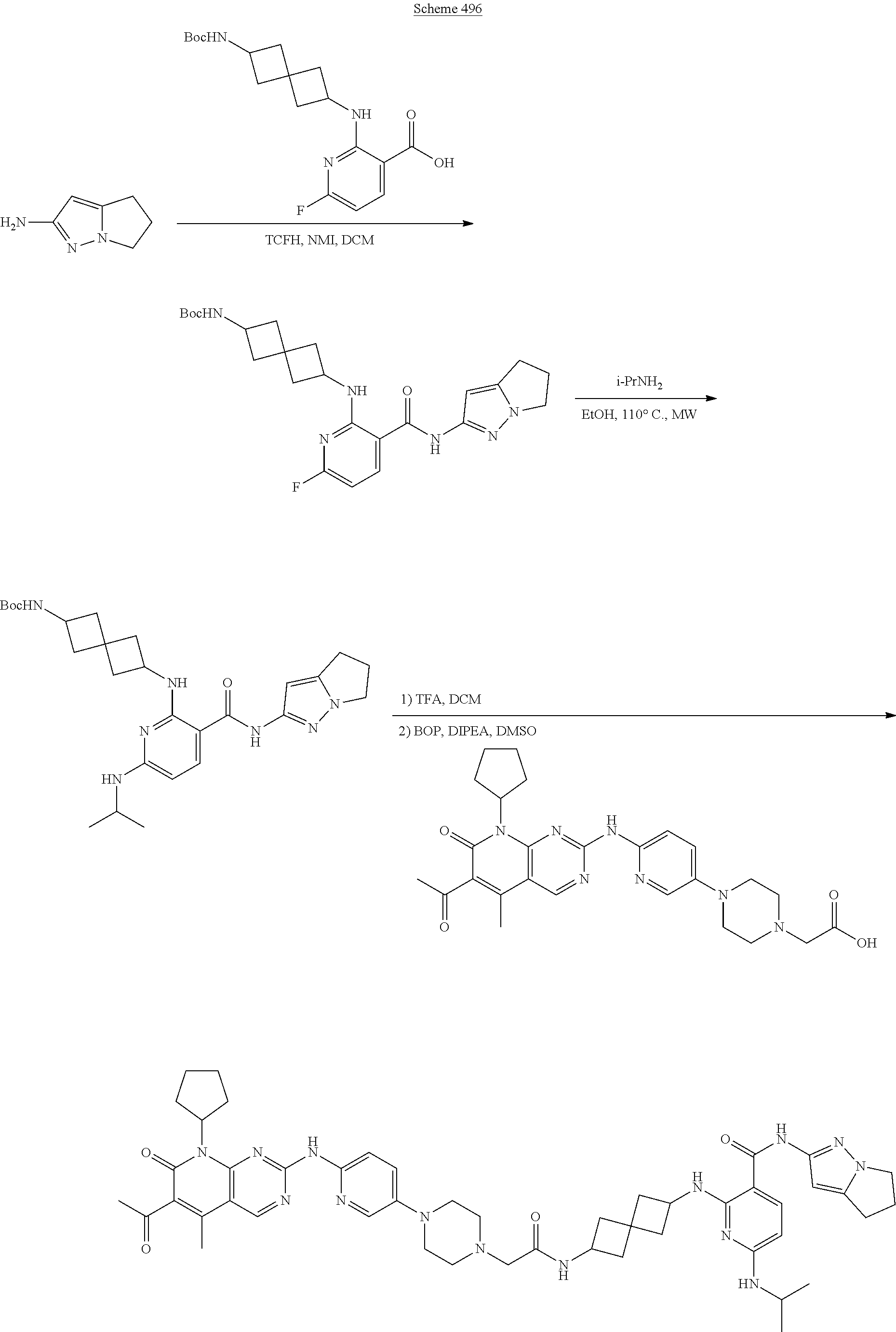

CPD-440 was synthesized following the standard procedures for preparing CPD-144 (13.0 mg, 9% yield over 4 steps) as a yellow solid. MS (ESI) m/z=897.7[M+H]⁺.

### Example 497. 2-((1-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)piperidin-4-yl)amino)-N-(6-(dimethylamino)pyridazin-3-yl)-6-((methyl-d3)amino)nicotinamide (CPD-441)

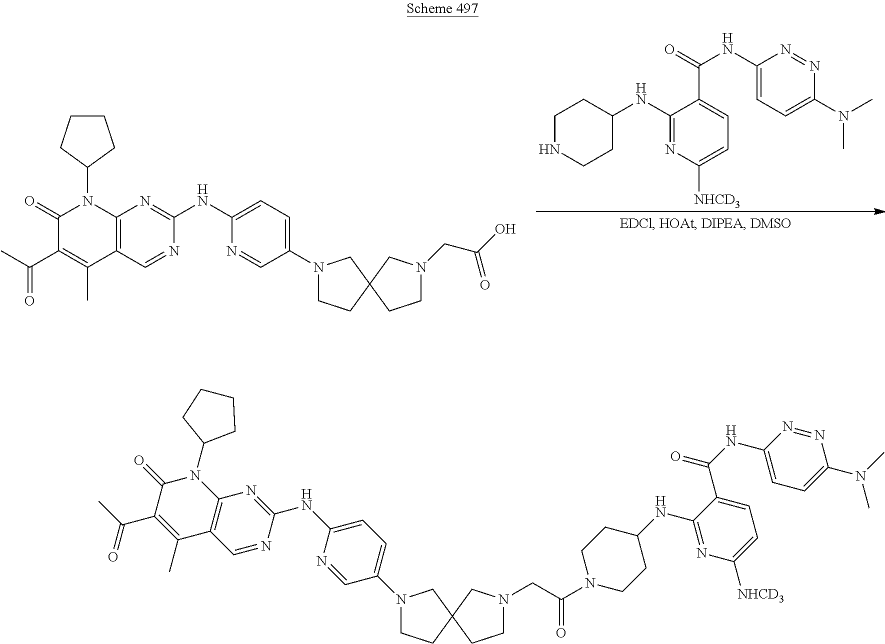

CPD-441 was synthesized following the standard procedure for preparing CPD-143 (1.5 mg, 31% yield) as a yellow solid. MS (ESI) m/z=902.1 [M+H]⁺.

### Example 498. 2-((1-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)azetidin-3-yl)amino)-N-(6-(dimethylamino)pyridazin-3-yl)-6-((methyl-d₃)amino)nicotinamide (CPD-442)

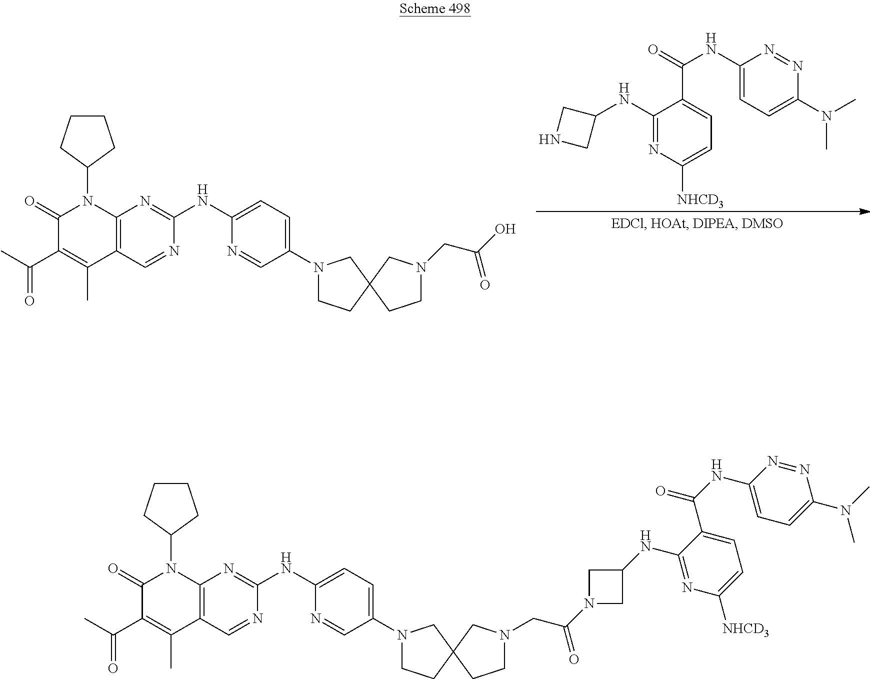

CPD-442 was synthesized following the standard procedure for preparing CPD-143 (3.1 mg, 25% yield) as a yellow solid. MS (ESI) m/z=873.8 [M+H]⁺.

### Example 499. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(2-cyclopropyl-1-methyl-1H-imidazol-4-yl)-6-((methyl-d₃)amino)nicotinamide (CPD-443)

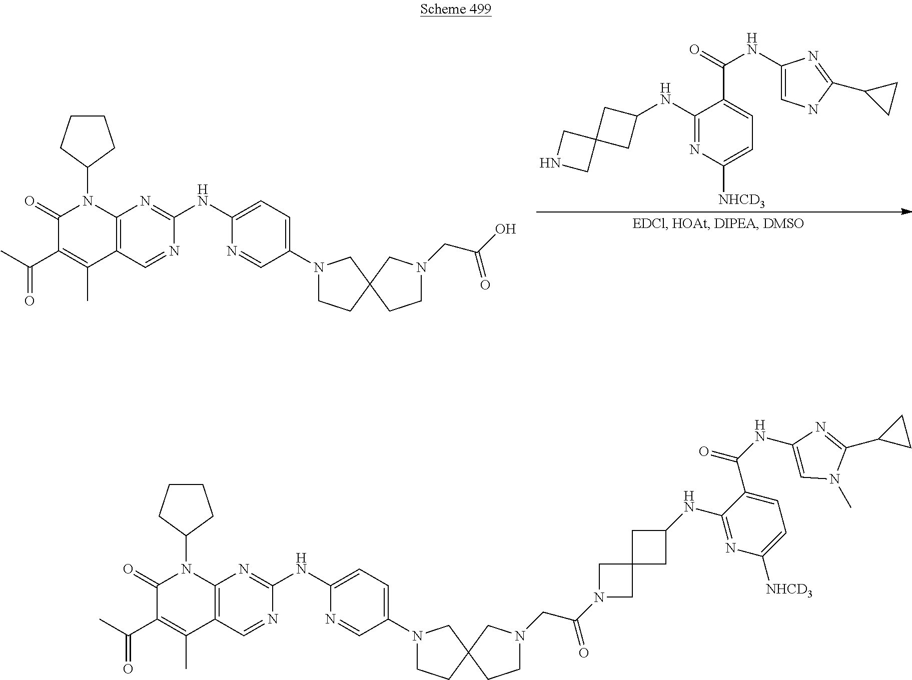

CPD-443 was synthesized following the standard procedure for preparing CPD-143 (2.0 mg, 37% yield) as a yellow solid. MS (ESI) m/z=912.8 [M+H]⁺.

### Example 500. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(2-cyclopropyl-1-methyl-1H-imidazol-4-yl)-6-(isopropylamino)nicotinamide (CPD-444)

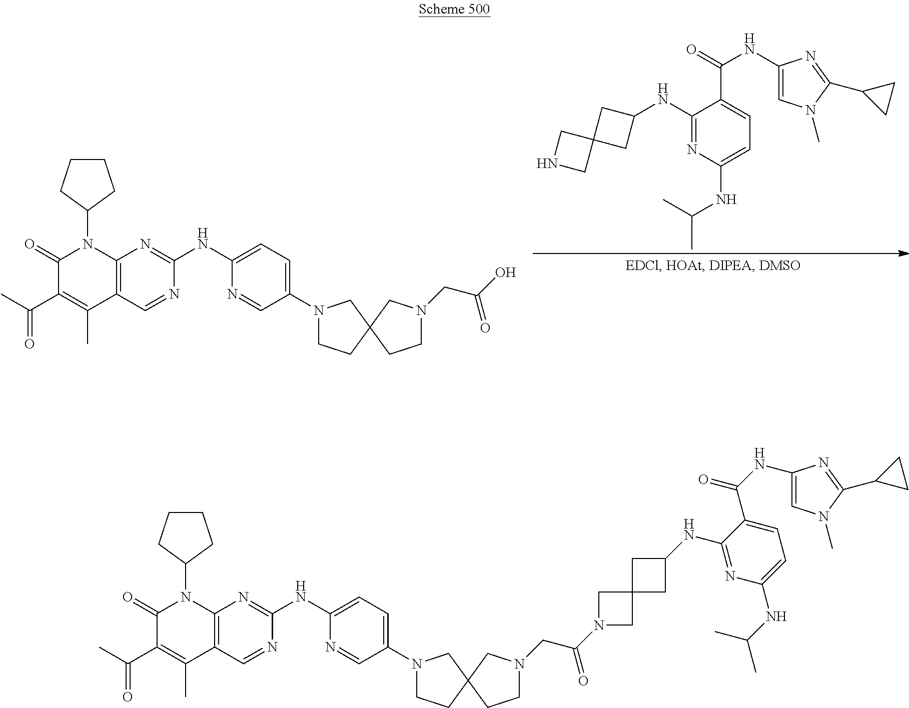 [SMILES: CC(=O)c1c(C)c2cnc(Nc3ccc(N4CCC5(CCN(CC(=O)O)C5)C4)cn3)nc2n(C2CCCC2)c1=O ; CC(C)Nc1ccc(C(=O)Nc2cn(C)c(C3CC3)n2)c(NC2CC3(CNC3)C2)n1 ; CC(=O)c1c(C)c2cnc(Nc3ccc(N4CCC5(CCN(CC(=O)N6CC7(CC(Nc8nc(NC(C)C)ccc8C(=O)Nc8cn(C)c(C9CC9)n8)C7)C6)C5)C4)cn3)nc2n(C2CCCC2)c1=O]

CPD-444 was synthesized following the standard procedure for preparing CPD-143 (1.7 mg, 31% yield) as a yellow solid. MS (ESI) m/z=937.8 [M+H]⁺.

### Example 501. 2-((3-((2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)methyl)cyclobutyl)amino)-N-(6-(dimethylamino)pyridazin-3-yl)-6-((methyl-d₃)amino)nicotinamide (CPD-445)

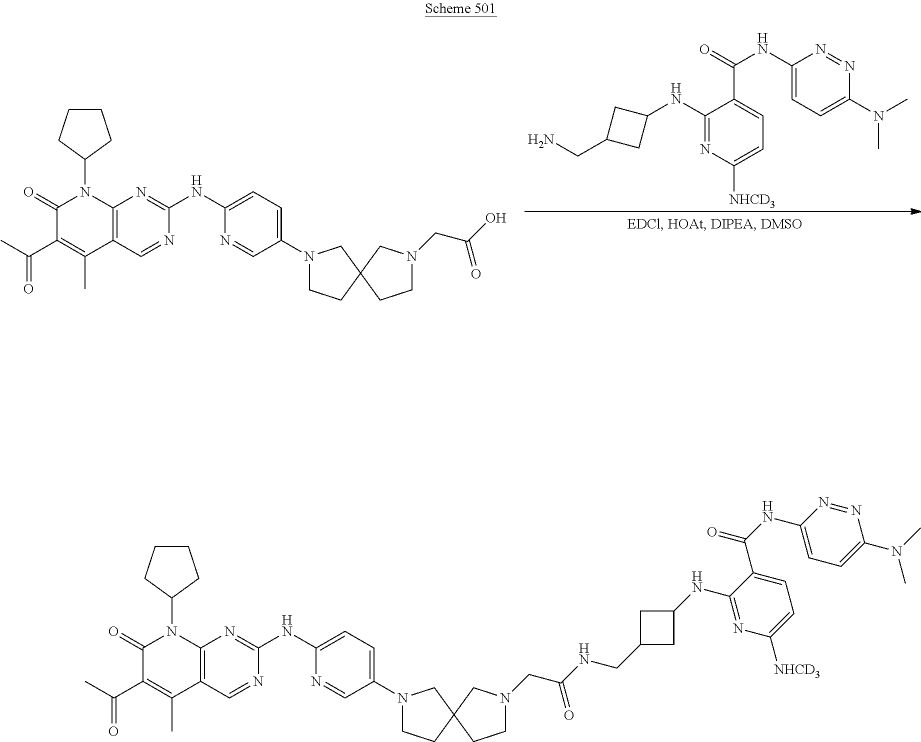

CPD-445 was synthesized following the standard procedure for preparing CPD-143 (1.9 mg, 39% yield) as a yellow solid. MS (ESI) m/z=901.8 [M+H]⁺.

### Example 502. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(1,5-dimethyl-1H-pyrazol-3-yl)-6-(isopropylamino)nicotinamide (CPD-446)

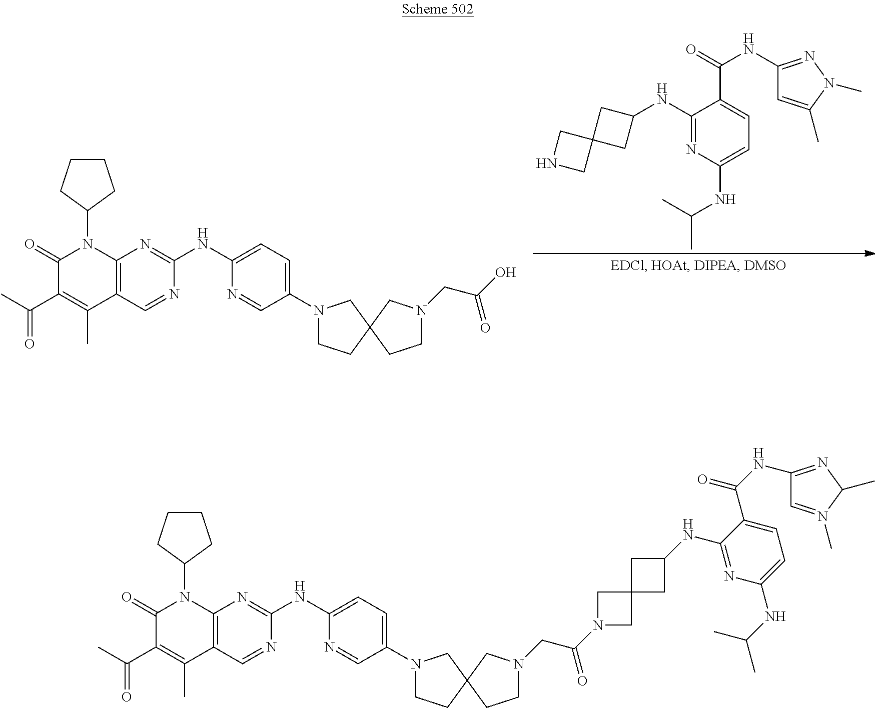

CPD-446 was synthesized following the standard procedure for preparing CPD-143 (1.6 mg, 30% yield) as a yellow solid. MS (ESI) m/z=912.1 [M+H]⁺.

### Example 503. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(1,5-dimethyl-1H-pyrazol-3-yl)-6-((methyl-d3)amino)nicotinamide (CPD-447)

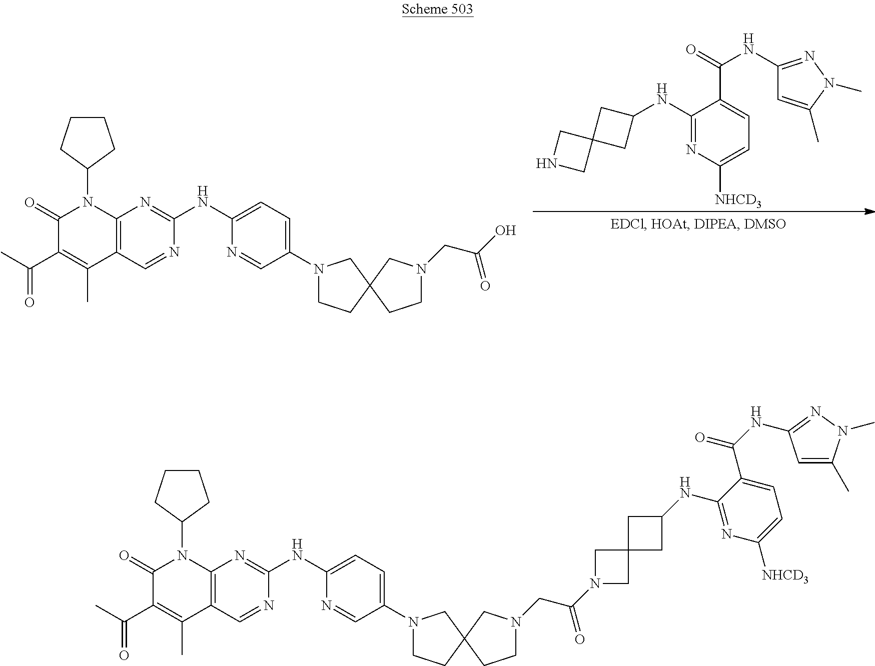

CPD-447 was synthesized following the standard procedure for preparing CPD-143 (1.2 mg, 26% yield) as a yellow solid. MS (ESI) m/z=886.7 [M+H]⁺.

### Example 504. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6-(dimethylamino)pyridazin-3-yl)-6-(isopropylamino)nicotinamide (CPD-448)

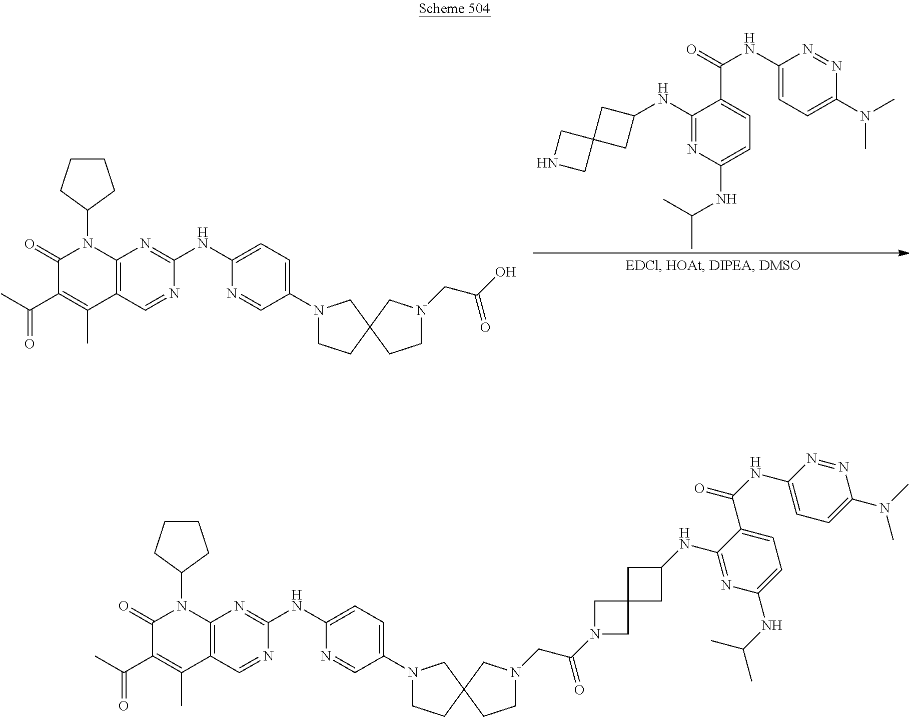 [SMILES: CC(=O)c1c(C)c2cnc(Nc3ccc(N4CCC5(CCN(CC(=O)O)C5)C4)cn3)nc2n(C2CCCC2)c1=O ; CC(C)Nc1ccc(C(=O)Nc2ccc(N(C)C)nn2)c(NC2CC3(CNC3)C2)n1 ; CC(=O)c1c(C)c2cnc(Nc3ccc(N4CCC5(CCN(CC(=O)N6CC7(CC(Nc8nc(NC(C)C)ccc8C(=O)Nc8ccc(N(C)C)nn8)C7)C6)C5)C4)cn3)nc2n(C2CCCC2)c1=O]

CPD-448 was synthesized following the standard procedure for preparing CPD-143 (1.2 mg, 23% yield) as a yellow solid. MS (ESI) m/z 939.1 [M+H]⁺.

### Example 505. 2-((6-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)spiro[3.3]heptan-2-yl)amino)-N-(6-(dimethylamino)pyridazin-3-yl)-6-((methyl-d₃)amino)nicotinamide (CPD-449)

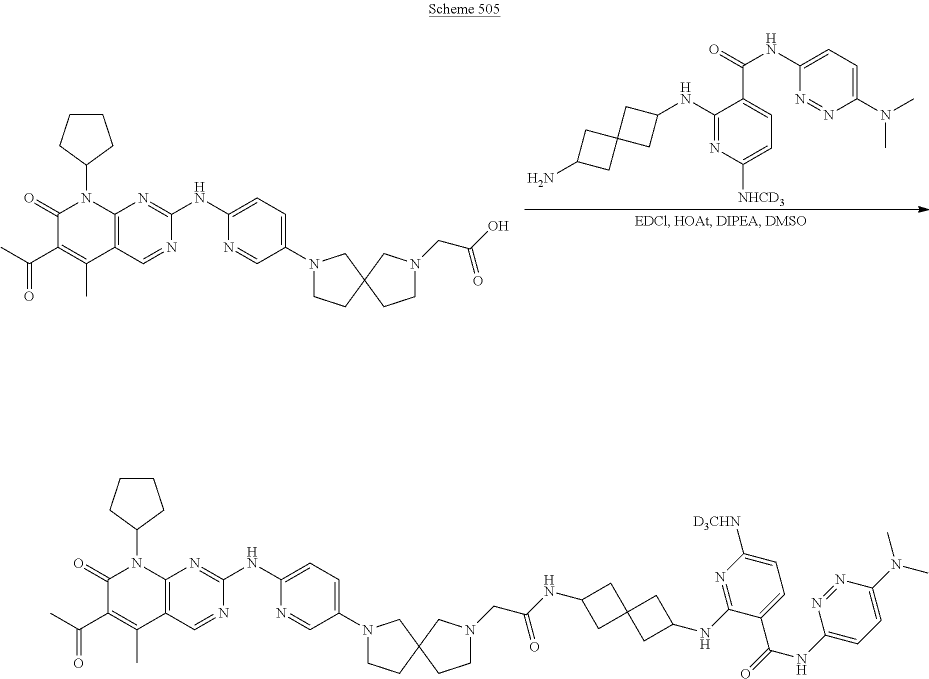

CPD-449 was synthesized following the standard procedure for preparing CPD-143 (1.8 mg, 34% yield) as a yellow solid. MS (ESI) m/z=928.1 [M+H]⁺.

### Example 506. 2-((3-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)cyclobutyl)amino)-N-(6-(dimethylamino)pyridazin-3-yl)-6-((methyl-d₃)amino)nicotinamide (CPD-450)

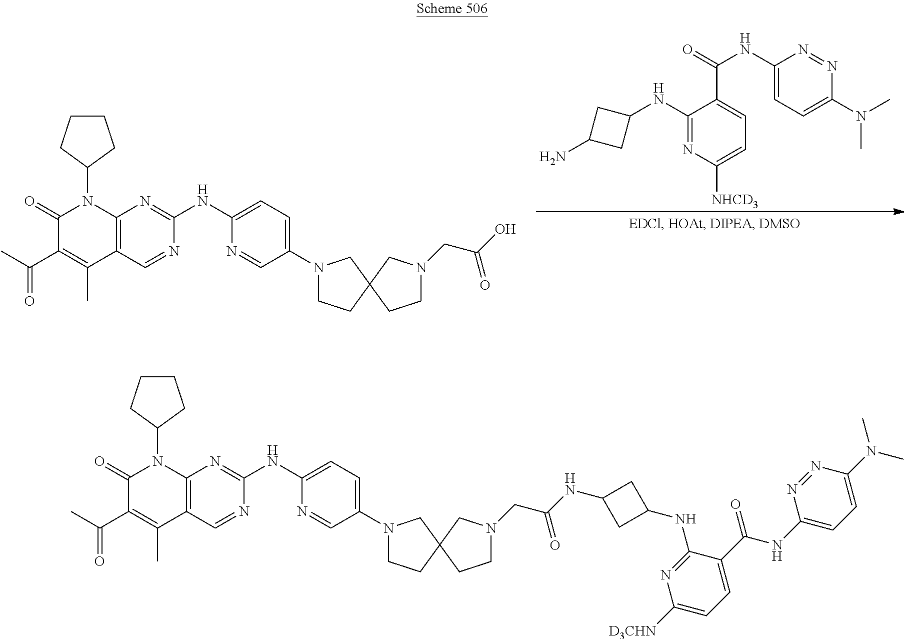

CPD-450 was synthesized following the standard procedure for preparing CPD-143 (1.3 mg, 25% yield) as a yellow solid. MS (ESI) m/z=888.0 [M+H]⁺.

### Example 507. 6-(6-((6-acetyl-8-cyclopentyl-5-methyl-7-oxo-7,8-dihydropyrido[2,3-d]pyrimidin-2-yl)amino)pyridin-3-yl)-N-(6-((3-((1,5-dimethyl-1H-pyrazol-3-yl)carbamoyl)-6-(isopropylamino)pyridin-2-yl)amino)spiro[3.3]heptan-2-yl)-6-azaspiro[2.5]octane-1-carboxamide (CPD-451)

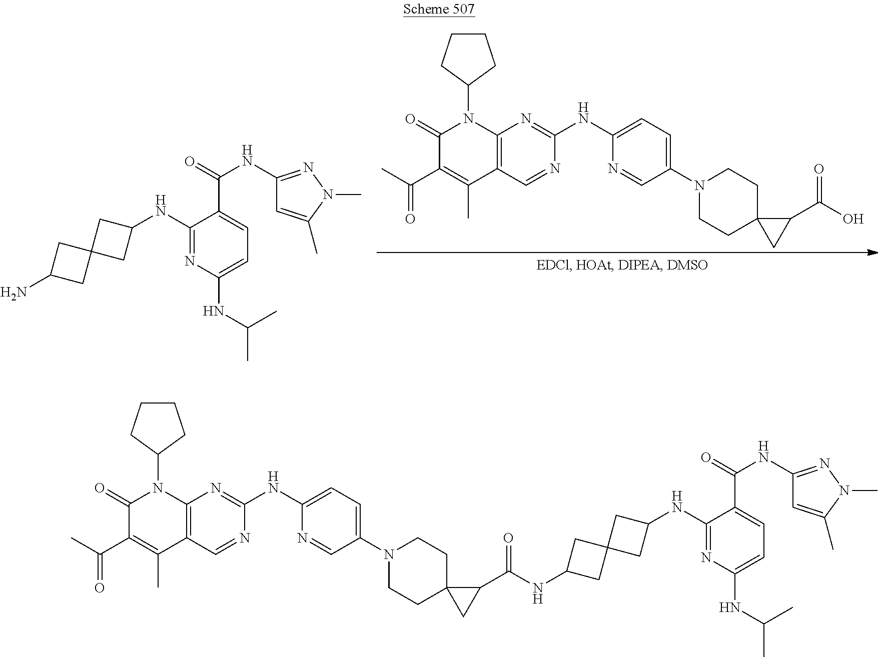

CPD-451 was synthesized following the standard procedure for preparing CPD-143 (2.1 mg, 25% yield) as a yellow solid. MS (ESI) m/z=896.8 [M+H]⁺.

### Example 508. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.31]heptan-2-yl)amino)-N-(1-cyclopropyl-5-methyl-1H-pyrazol-3-yl)-6-(isopropylamino)nicotinamide (CPD-452)

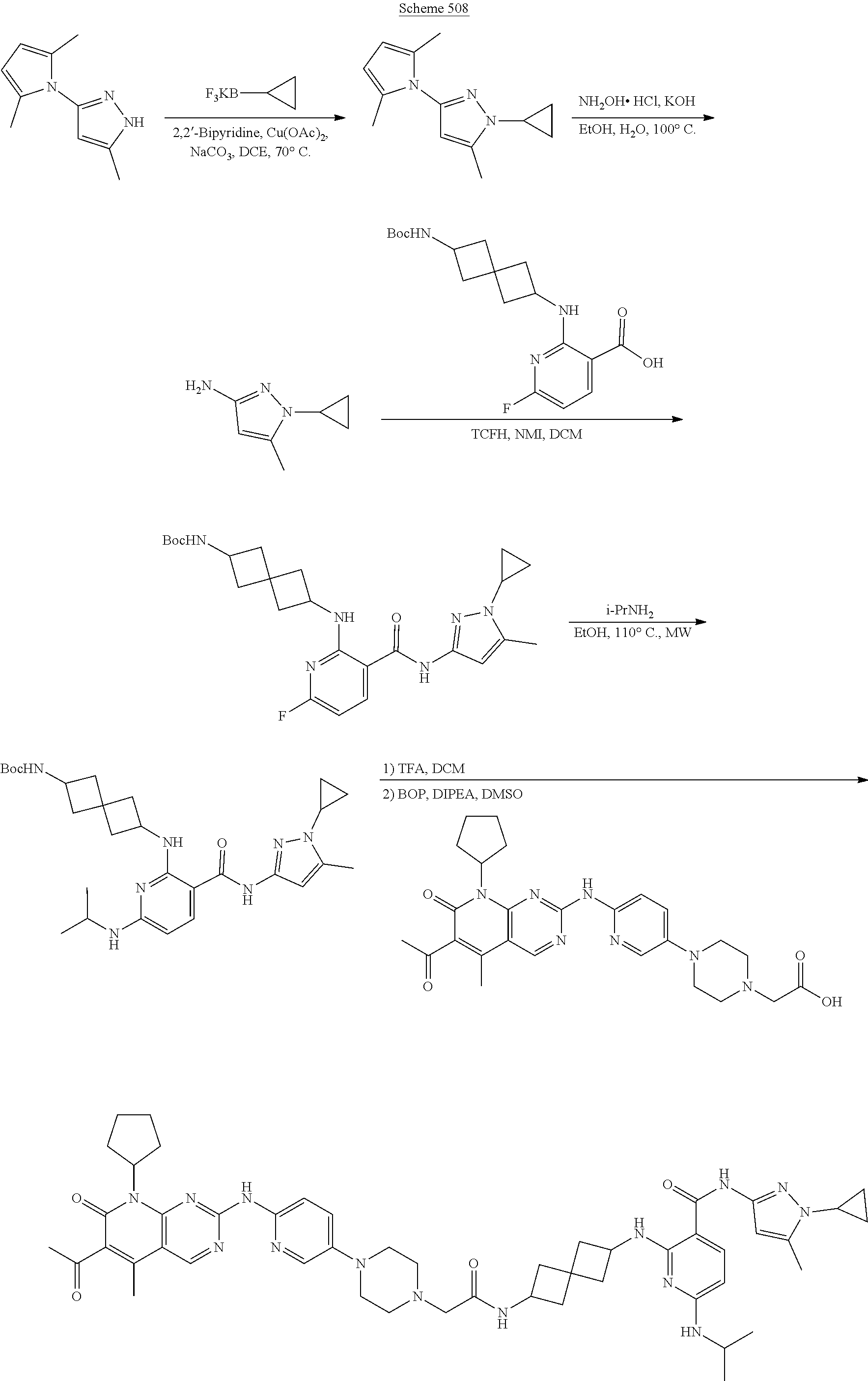

### Step 1. Synthesis of 1-cyclopropyl-3-(2,5-dimethyl-1H-pyrrol-1-yl)-5-methyl-1H-pyrazole

To a solution of 3-(2,5-dimethyl-1H-pyrrol-1-yl)-5-methyl-1H-pyrazole (400 mg, 2.28 mmol) in DCE (15 mL) were added potassium cyclopropyltrifluoroborate (338 mg, 2.28 mmol), Na₂CO₃ (242 mg, 2.28 mmol), 2,2′-Bipyridine (356 mg, 2.28 mmol) and Cu(OAc)₂ (415 mg, 2.28 mmol). The reaction mixture was stirred at 70° C. for 6 h. After cooling down to rt, the mixture was diluted with water and extracted with ethyl acetate (2×50 mL). The combined organic layers were washed with saturated brine (50 mL), dried over anhydrous sodium sulfate, filtered, and concentrated. The crude product was purified by silica gel column chromatography to give the desired product (250 mg, 510% yield) as a light-yellow solid. MS (ESI) m/z=216.6 [M+H]⁺.

### Step 2. Synthesis of 1-cyclopropyl-5-methyl-1H-pyrazol-3-amine

To a solution of 1-cyclopropyl-3-(2,5-dimethyl-1H-pyrrol-1-yl)-5-methyl-1H-pyrazole (250 mg, 1.16 mmol) in EtOH (8 mL) and water (4 mL) were added NH₂OH·HCl (240 mg, 3.48 mmol) and KOH (195 mg, 3.48 mmol). The reaction mixture was stirred at 100° C. for 48 h. The reaction mixture was poured into water (30 mL) and extracted with ethyl acetate (3×30 mL). The combined organic layers were washed with saturated brine (40 mL), dried over anhydrous sodium sulfate, filtered, and concentrated. The residue was purified by silica gel chromatography to give the desired product (100 mg, 63% yield) as a light-yellow solid. MS(ESI) m/z=138.1 [M+H]⁺.

The remaining steps were performed according to the procedures for preparing CPD-144 to give the desired product (10 mg, 110% yield for 4 steps) as a yellow solid. MS (ESI) m/z=911.8 [M+H]⁺.

### Example 509. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(6-methoxypyridin-2-yl)nicotinamide (CPD-453)

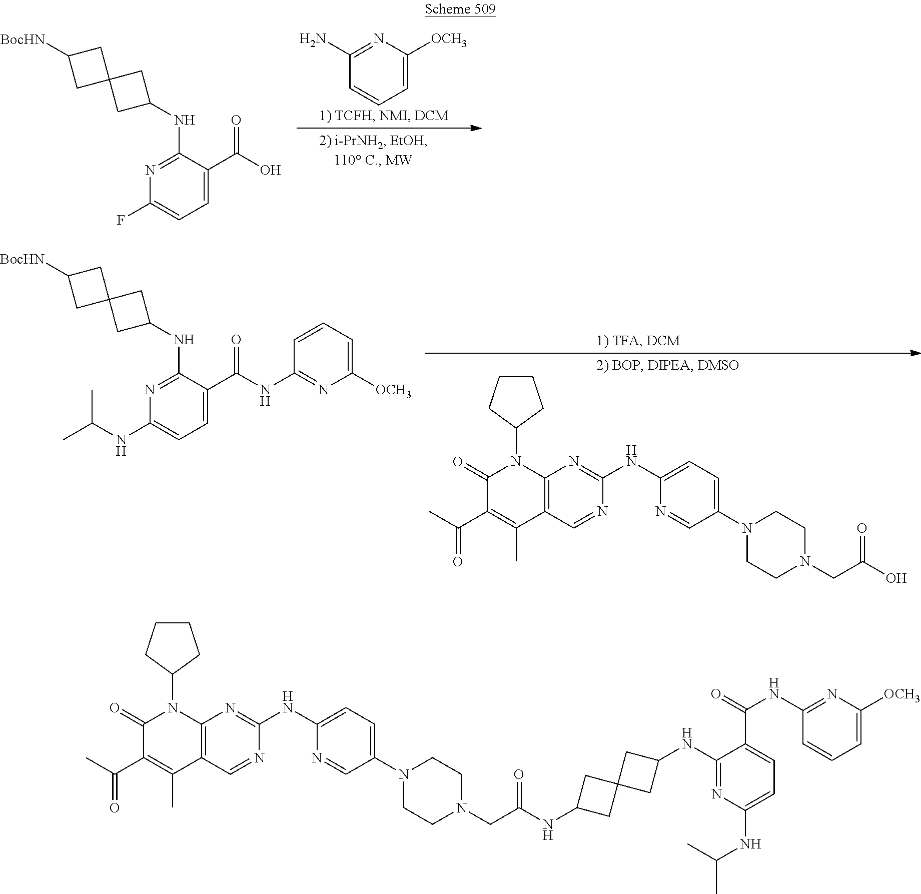 [SMILES: CC(C)(C)OC(=O)NC1CC2(C1)CC(Nc1nc(F)ccc1C(=O)O)C2 ; COc1cccc(N)n1 ; COc1cccc(NC(=O)c2ccc(NC(C)C)nc2NC2CC3(CC(NC(=O)OC(C)(C)C)C3)C2)n1 ; CC(=O)c1c(C)c2cnc(Nc3ccc(N4CCN(CC(=O)O)CC4)cn3)nc2n(C2CCCC2)c1=O ; COc1cccc(NC(=O)c2ccc(NC(C)C)nc2NC2CC3(CC(NC(=O)CN4CCN(c5ccc(Nc6ncc7c(C)c(C(C)=O)c(=O)n(C8CCCC8)c7n6)nc5)CC4)C3)C2)n1]

CPD-453 was synthesized following the standard procedures for preparing CPD-144 (10.0 mg, 10% yield) as a yellow solid. MS (ESI) m/z=898.7 [M+H]⁺.

### Example 510. 2-((1-(3-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)propanoyl)piperidin-4-yl)amino)-N-(6-(dimethylamino)pyridazin-3-yl)-6-(isopropylamino)nicotinamide (CPD-454)

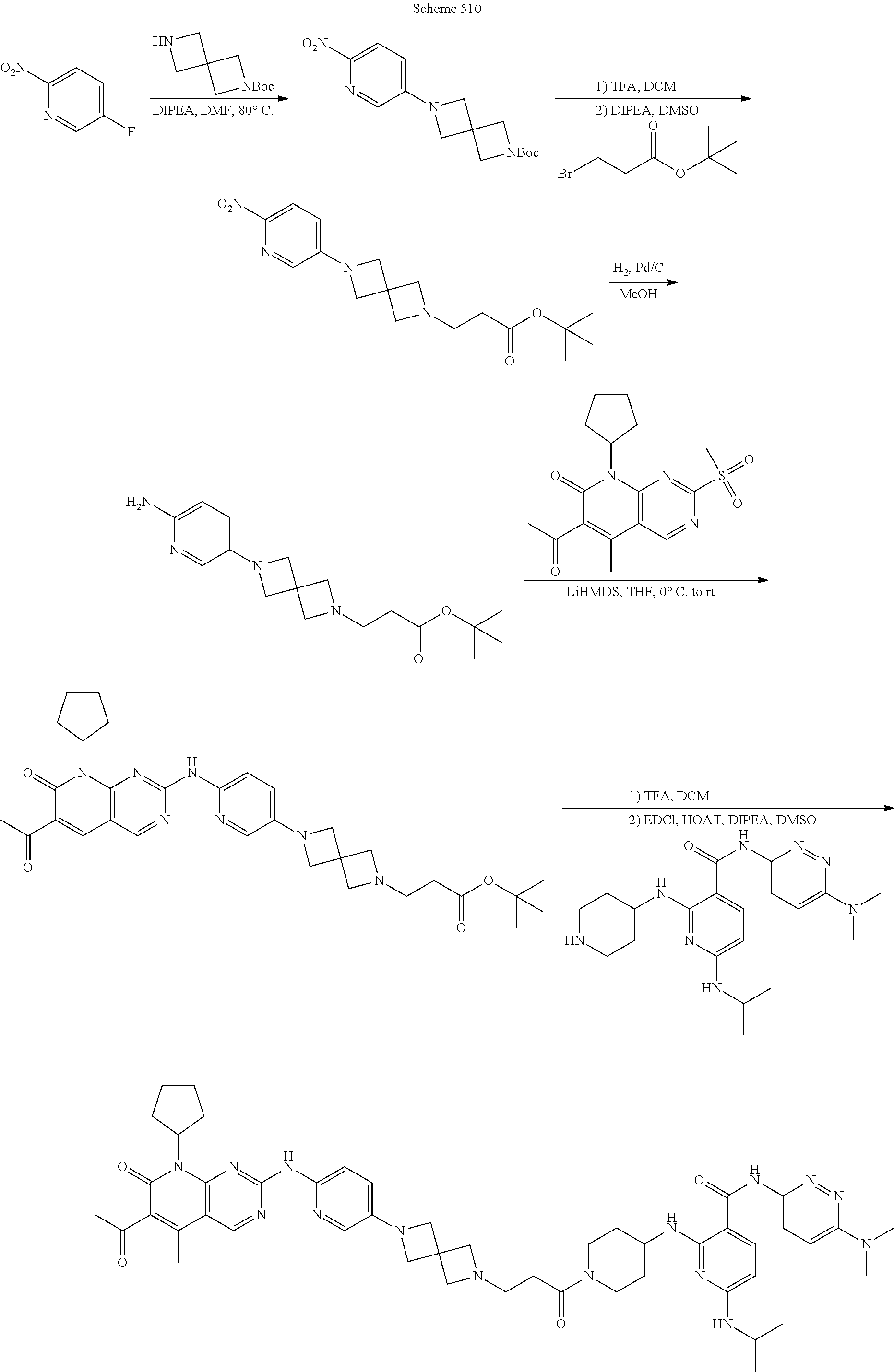

CPD-454 was synthesized following the standard procedures for preparing CPD-418 (4.4 mg, 3% yield over 7 steps) as a yellow solid. MS (ESI) m/z=912.8 [M+H]⁺.

### Example 511. 2-((2-(3-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)propanoyl)-2-azaspiro[3.3]heptan-6-yl)amino)-N-(6-(dimethylamino)pyridazin-3-yl)-6-(isopropylamino)nicotinamide (CPD-455)

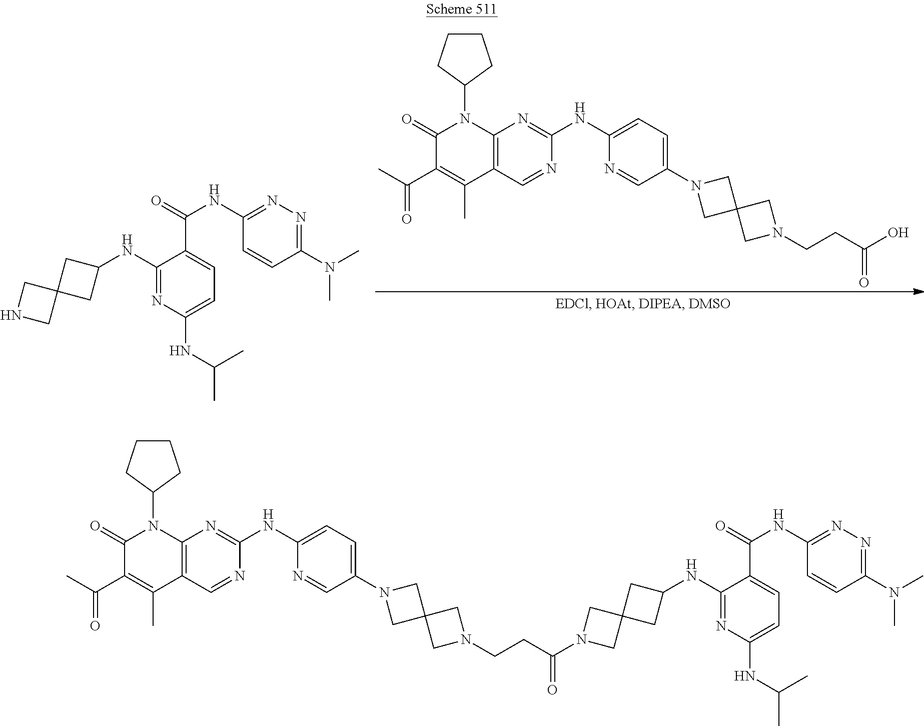 [SMILES: CC(C)Nc1ccc(C(=O)Nc2ccc(N(C)C)nn2)c(NC2CC3(CNC3)C2)n1 ; CC(=O)c1c(C)c2cnc(Nc3ccc(N4CC5(CN(CCC(=O)O)C5)C4)cn3)nc2n(C2CCCC2)c1=O ; CC(=O)c1c(C)c2cnc(Nc3ccc(N4CC5(CN(CCC(=O)N6CC7(CC(Nc8nc(NC(C)C)ccc8C(=O)Nc8ccc(N(C)C)nn8)C7)C6)C5)C4)cn3)nc2n(C2CCCC2)c1=O]

CPD-455 was synthesized following the standard procedure for preparing CPD-143 (2.2 mg, 10% yield) as a yellow solid. MS (ESI) m/z=924.8 [M+H]⁺.

### Example 512. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanoyl)-2-azaspiro[3.3]heptan-6-yl)amino)-N-(6-(dimethylamino)pyridazin-3-yl)-6-((methyl-d3)amino)nicotinamide (CPD-456)

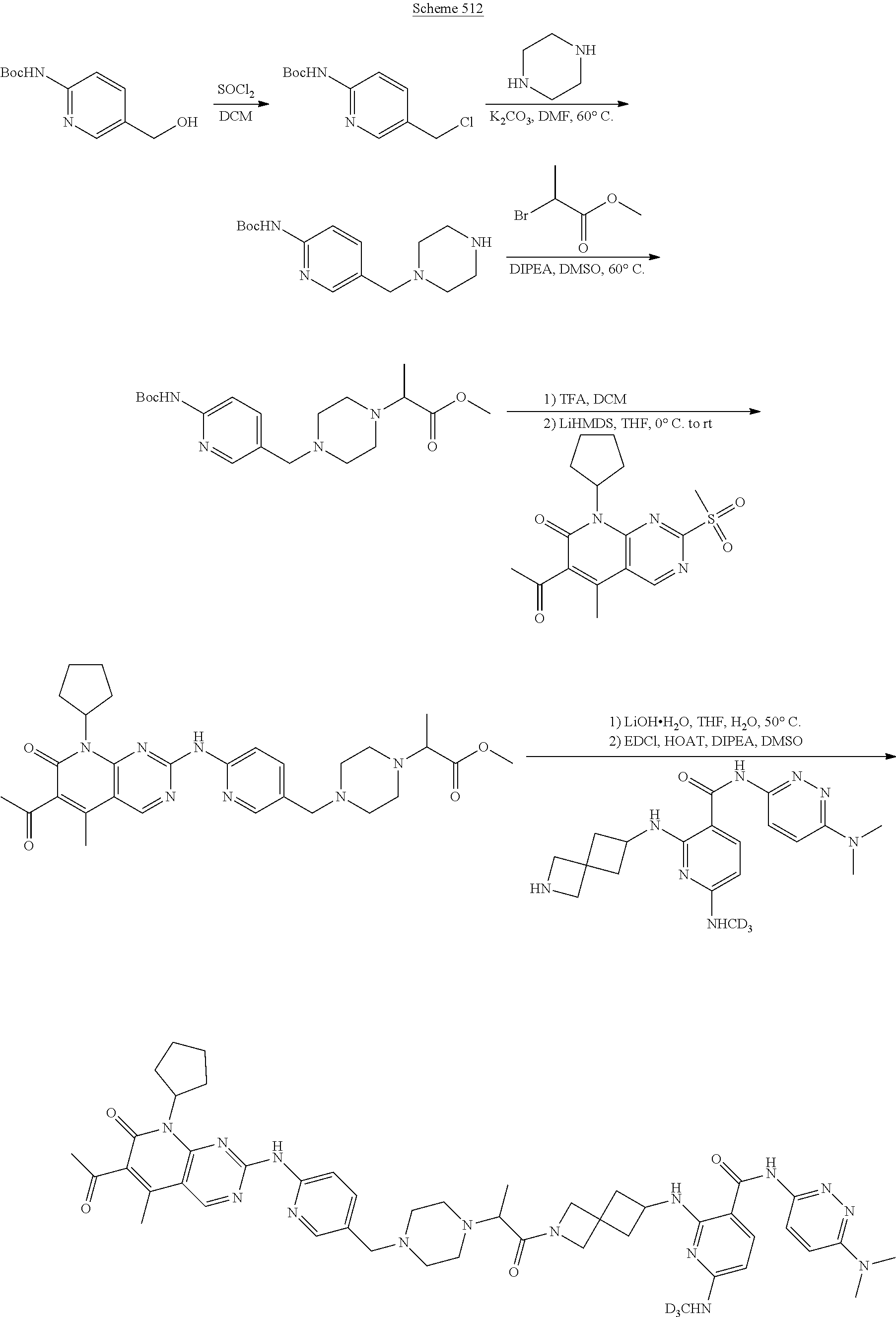

CPD-456 was synthesized following the standard procedures for preparing CPD-392 (3.8 mg, 3% yield over 7 steps) as a yellow solid. MS (ESI) m/z=901.8 [M+H]⁺.

### Example 513. 6-(6-((6-acetyl-8-cyclopentyl-5-methyl-7-oxo-7,8-dihydropyrido[2,3-d]pyrimidin-2-yl)amino)pyridin-3-yl)-N-(6-((3-((2-cyclopropyl-1-methyl-1H-imidazol-4-yl)carbamoyl)-6-(isopropylamino)pyridin-2-yl)amino)spiro[3.3]heptan-2-yl)-6-azaspiro[2.5]octane-1-carboxamide (CPD-457)

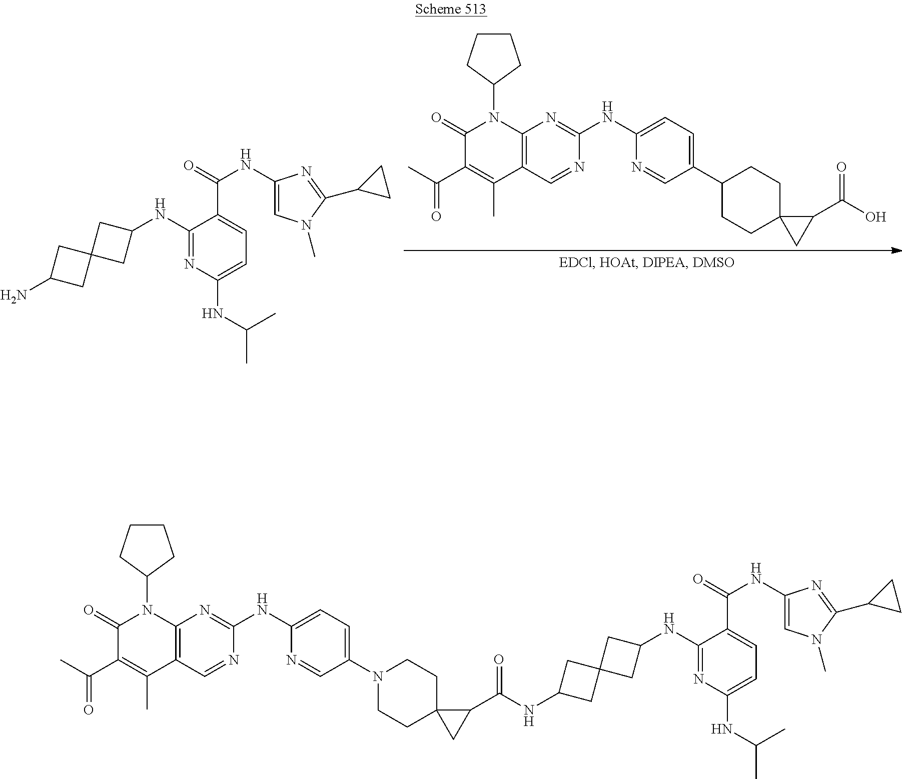

CPD-457 was synthesized following the standard procedure for preparing CPD-143 (4.1 mg, 10% yield) as a yellow solid. MS (ESI) m/z=923.1 [M+H]⁺.

### Example 514. 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-((methyl-d₃)amino)nicotinamide (CPD-458)

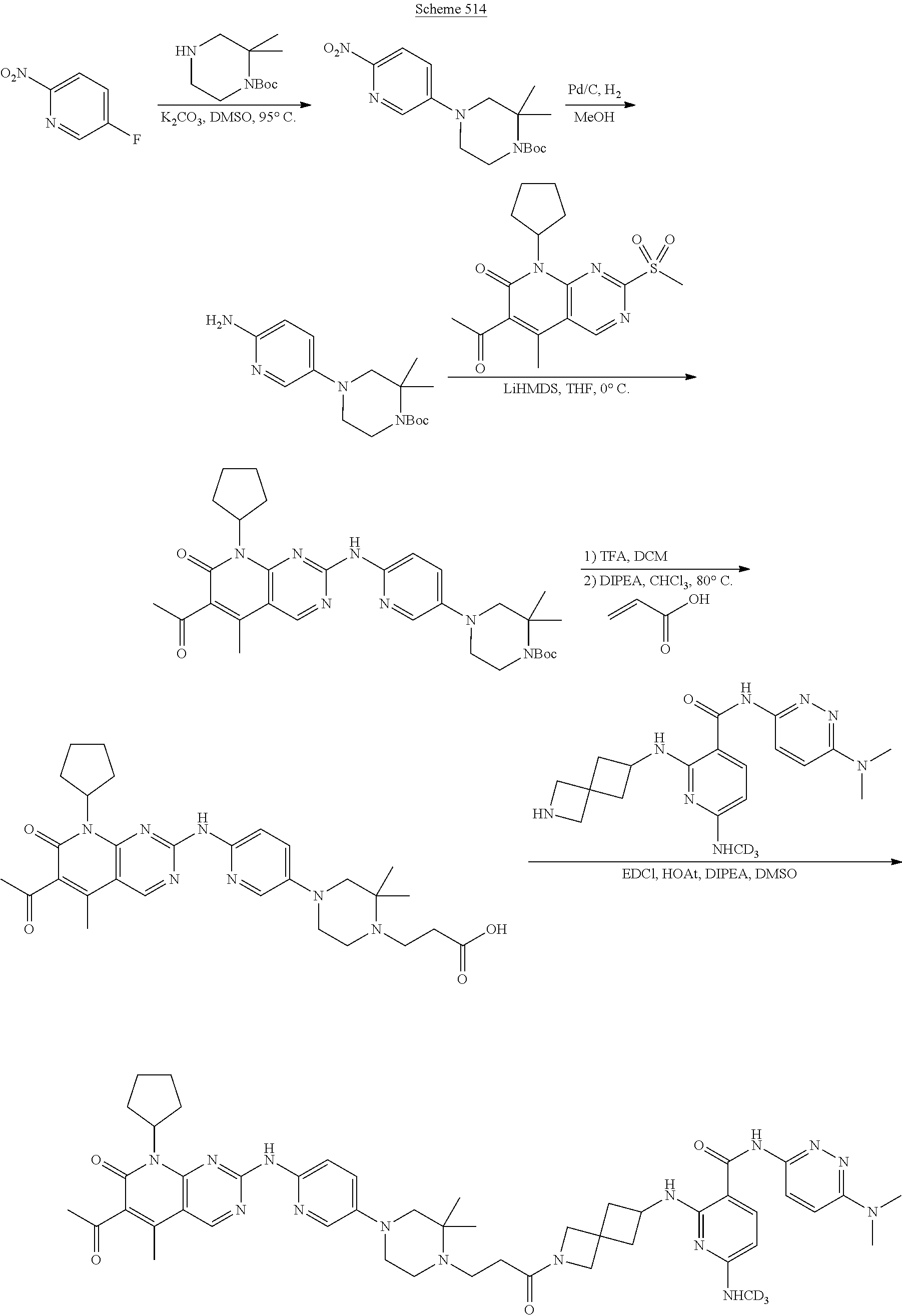

CPD-458 was synthesized following the standard procedures for preparing CPD-364 (2.8 mg, 2% yield over 6 steps) as a yellow solid. MS (ESI) m/z=915.8 [M+H]⁺.

### Example 515. 2-((2-(3-(4-(6-((6-acetyl-8-cyclopentyl-5-methyl-7-oxo-7,8-dihydropyrido[2,3-d]pyrimidin-2-yl)amino)pyridin-3-yl)-2,2-dimethylpiperazin-1-yl)propanoyl)-2-azaspiro[3.3]heptan-6-yl)amino)-N-(6-(dimethylamino)pyridazin-3-yl)-6-(isopropylamino)nicotinamide (CPD-459)

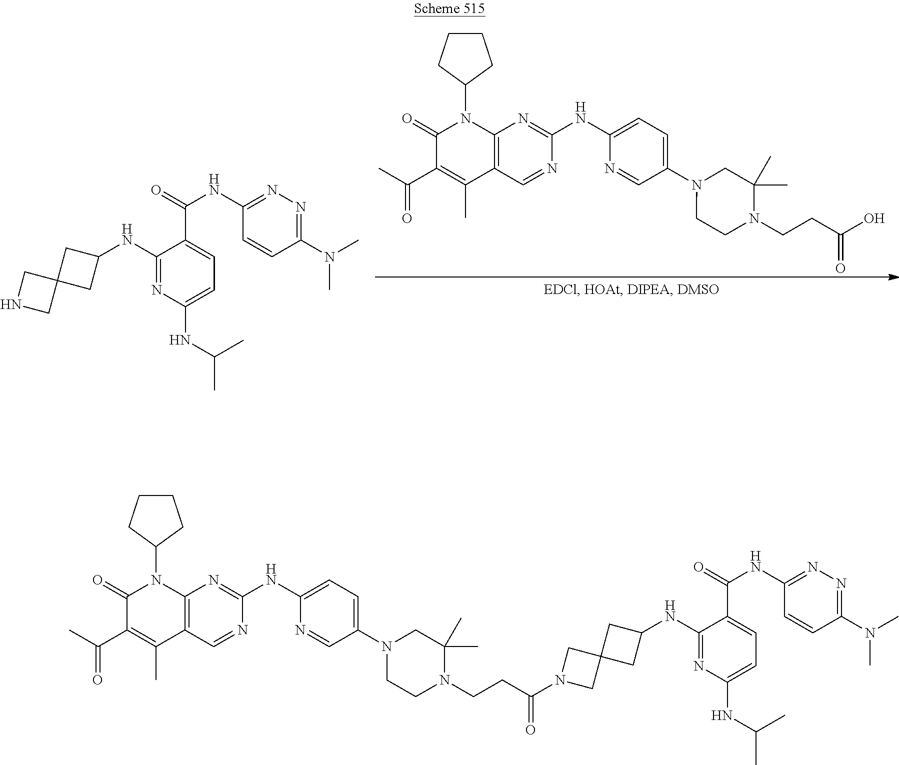 [SMILES: CC(C)Nc1ccc(C(=O)Nc2ccc(N(C)C)nn2)c(NC2CC3(CNC3)C2)n1 ; CC(=O)c1c(C)c2cnc(Nc3ccc(N4CCN(CCC(=O)O)C(C)(C)C4)cn3)nc2n(C2CCCC2)c1=O ; CC(=O)c1c(C)c2cnc(Nc3ccc(N4CCN(CCC(=O)N5CC6(CC(Nc7nc(NC(C)C)ccc7C(=O)Nc7ccc(N(C)C)nn7)C6)C5)C(C)(C)C4)cn3)nc2n(C2CCCC2)c1=O]

CPD-459 was synthesized following the standard procedure for preparing CPD-143 (1.9 mg, 22% yield) as a yellow solid. MS (ESI) m/z=940.8 [M+H]⁺.

### Example 516. 2-((3-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)cyclobutyl)amino)-N-(6-(dimethylamino)pyridazin-3-yl)-6-(isopropylamino)nicotinamide (CPD-460)

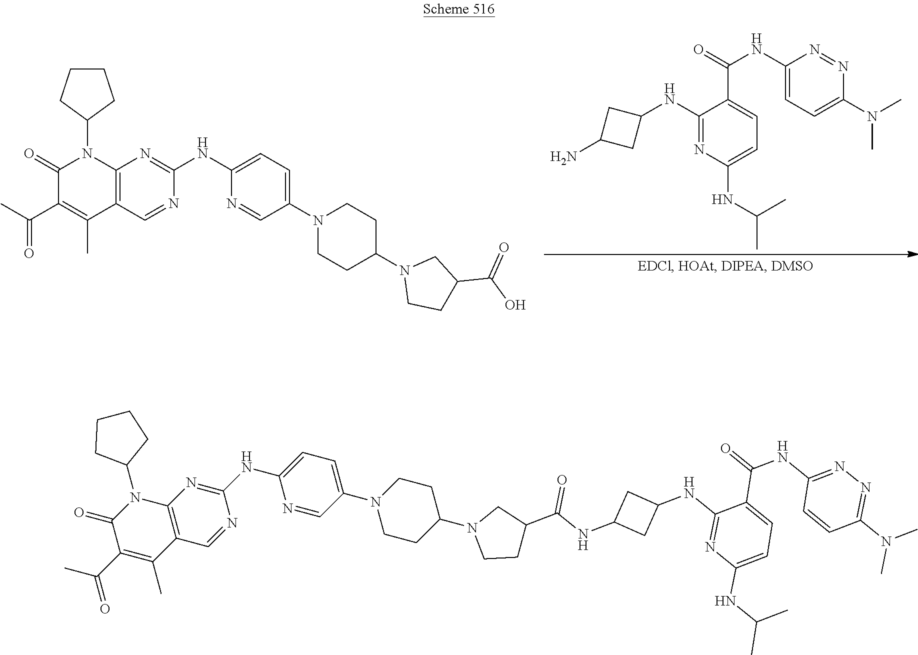 [SMILES: CC(=O)c1c(C)c2cnc(Nc3ccc(N4CCC(N5CCC(C(=O)O)C5)CC4)cn3)nc2n(C2CCCC2)c1=O ; CC(C)Nc1ccc(C(=O)Nc2ccc(N(C)C)nn2)c(NC2CC(N)C2)n1 ; CC(=O)c1c(C)c2cnc(Nc3ccc(N4CCC(N5CCC(C(=O)NC6CC(Nc7nc(NC(C)C)ccc7C(=O)Nc7ccc(N(C)C)nn7)C6)C5)CC4)cn3)nc2n(C2CCCC2)c1=O]

CPD-460 was synthesized following the standard procedure for preparing CPD-143 (1.6 mg, 16% yield) as a yellow solid. MS (ESI) m/z=927.1 [M+H]⁺.

### Example 517. 2-((3-((1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-3-carboxamido)methyl)cyclobutyl)amino)-N-(6-(dimethylamino)pyridazin-3-yl)-6-(isopropylamino)nicotinamide (CPD-461)

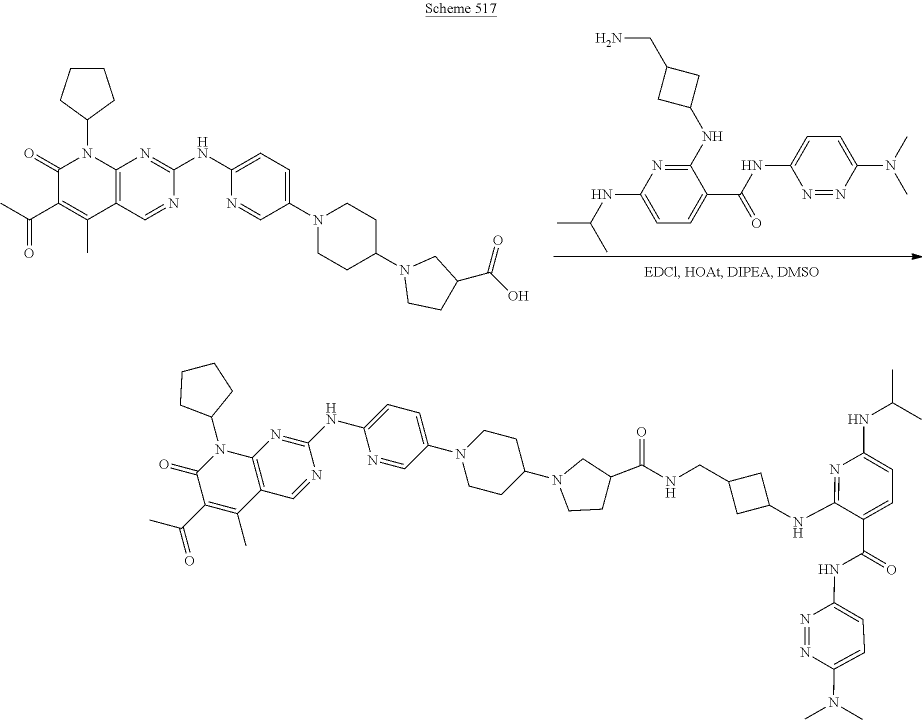 [SMILES: CC(=O)c1c(C)c2cnc(Nc3ccc(N4CCC(N5CCC(C(=O)O)C5)CC4)cn3)nc2n(C2CCCC2)c1=O ; CC(C)Nc1ccc(C(=O)Nc2ccc(N(C)C)nn2)c(NC2CC(CN)C2)n1 ; CC(=O)c1c(C)c2cnc(Nc3ccc(N4CCC(N5CCC(C(=O)NCC6CC(Nc7nc(NC(C)C)ccc7C(=O)Nc7ccc(N(C)C)nn7)C6)C5)CC4)cn3)nc2n(C2CCCC2)c1=O]

CPD-461 was synthesized following the standard procedure for preparing CPD-143 (1.5 mg, 14% yield) as a yellow solid. MS (ESI) m/z=941.1 [M+H]⁺.

### Example 518. 2-((4-(1-(1-(6-((6-acetyl-8-cyclopentyl-5-menthyl-7-oxo-7,8-dihydropyrido[2,3-d]pyrimidin-2-yl)amino)pyridin-3-yl)piperidin-4-yl)pyrrolidine-3-carboxamido)cyclohexyl)amino)-N-(6-(dimethylamino)pyridazin-3-yl)-6-(isopropylamino)nicotinamide (CPD-462)

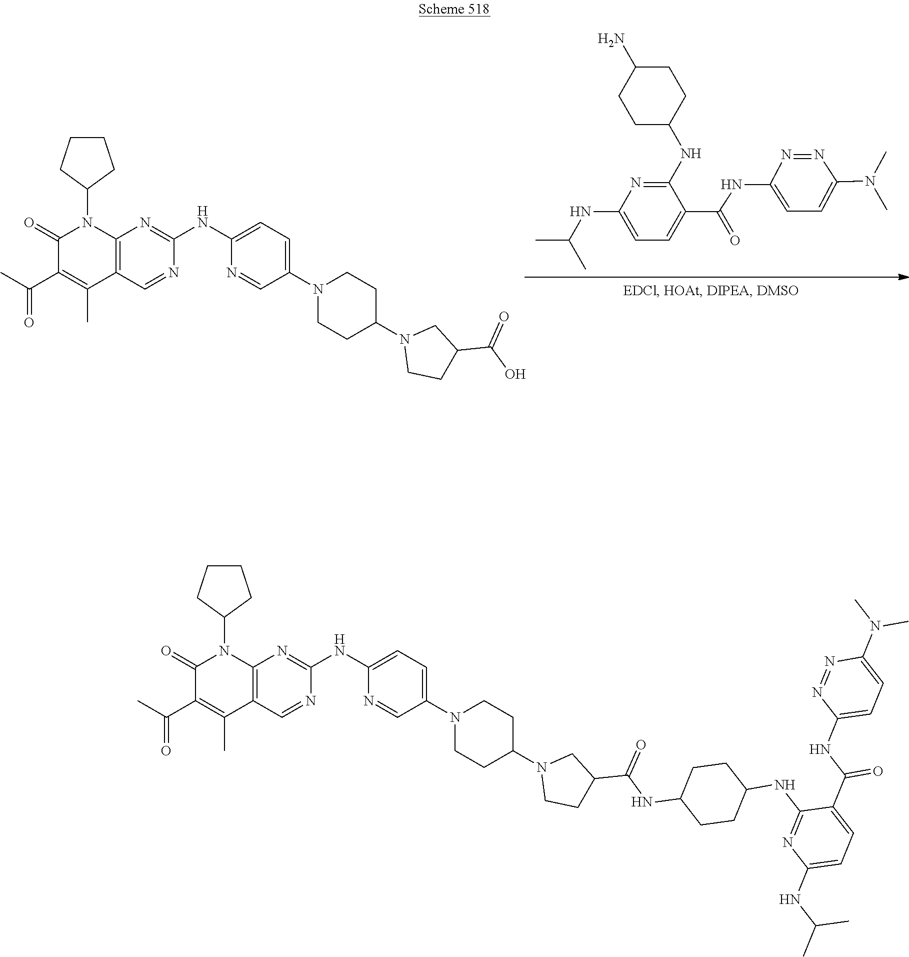 [SMILES: CC(=O)c1c(C)c2cnc(Nc3ccc(N4CCC(N5CCC(C(=O)O)C5)CC4)cn3)nc2n(C2CCCC2)c1=O ; CC(C)Nc1ccc(C(=O)Nc2ccc(N(C)C)nn2)c(NC2CCC(N)CC2)n1 ; CC(=O)c1c(C)c2cnc(Nc3ccc(N4CCC(N5CCC(C(=O)NC6CCC(Nc7nc(NC(C)C)ccc7C(=O)Nc7ccc(N(C)C)nn7)CC6)C5)CC4)cn3)nc2n(C2CCCC2)c1=O]

CPD-462 was synthesized following the standard procedure for preparing CPD-143 (1.2 mg, 15% yield) as a yellow solid. MS (ESI) m/z=955.1 [M+H]⁺.

### Example 519. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(6-(dimethylamino)pyridazin-3-yl)-6-((methyl-d₃)amino)nicotinamide (CPD-463)

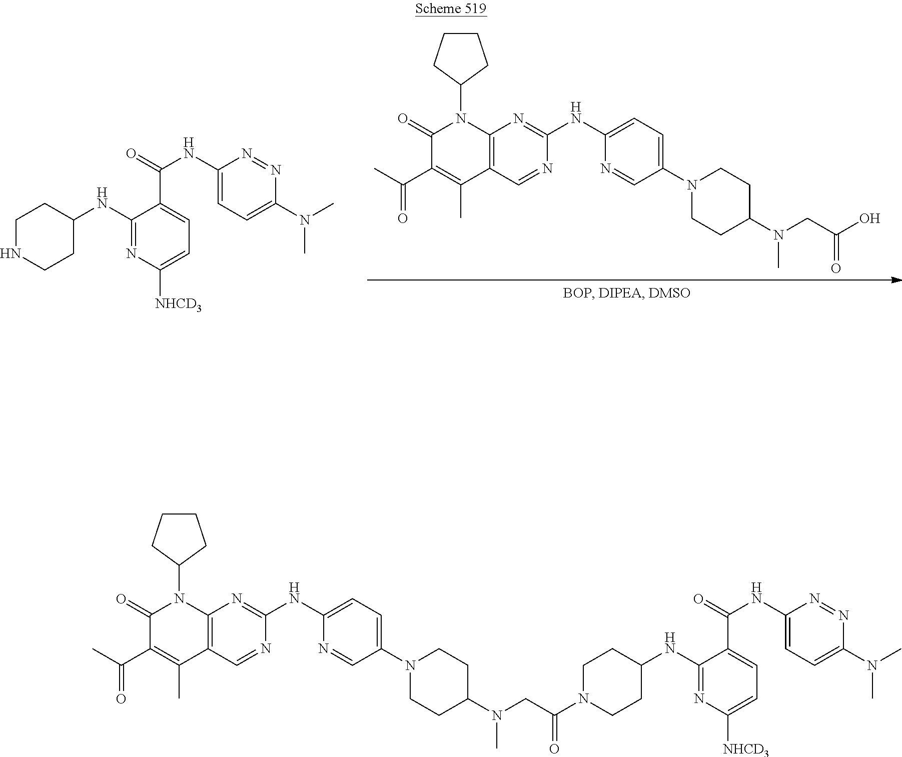

CPD-463 was synthesized following the standard procedure for preparing CPD-261 (8.0 mg, 48% yield) as a yellow solid. MS (ESI) m/z=889.7 [M+H]⁺.

### Example 520. 2-((2-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)-2-azaspiro[3.3]heptan-6-yl)amino)-N-(6-(dimethylamino)pyridazin-3-yl)-6-((methyl-d₃)amino)nicotinamide (CPD-464)

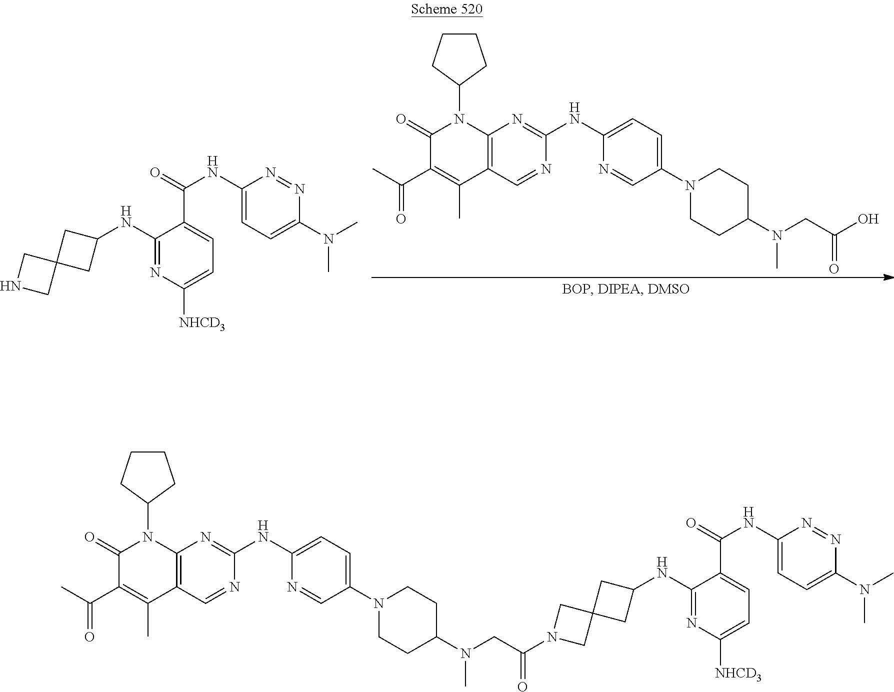

CPD-464 was synthesized following the standard procedure for preparing CPD-261 (7.0 mg, 36% yield) as a yellow solid. MS (ESI) m/z=901.7 [M+H]⁺.

### Example 521. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(1,5-dimethyl-1H-pyrazol-3-yl)-6-(isopropylamino)nicotinamide (CPD-465)

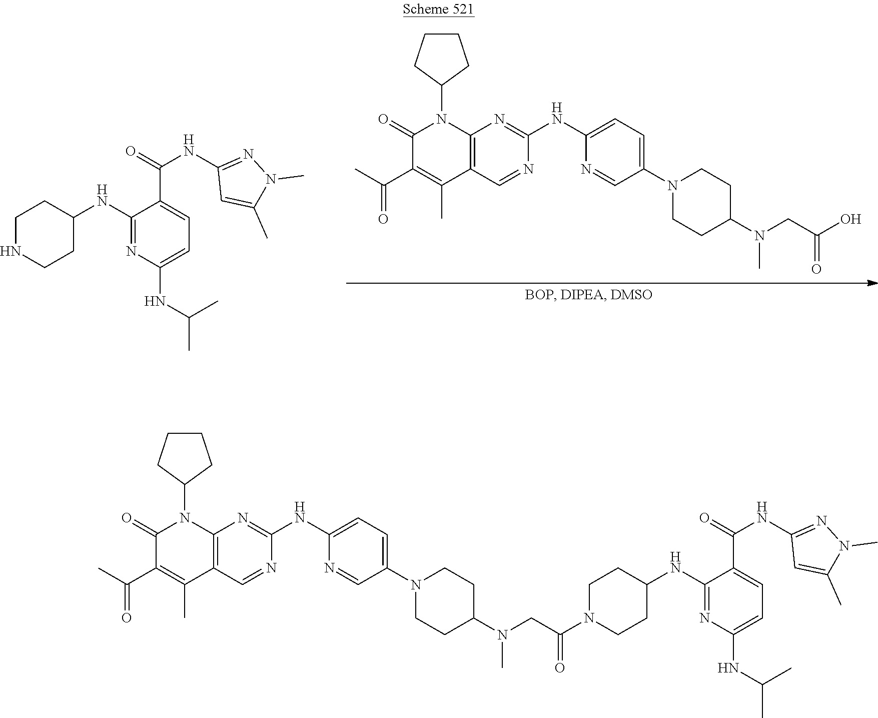 [SMILES: Cc1cc(NC(=O)c2ccc(NC(C)C)nc2NC2CCNCC2)nn1C ; CC(=O)c1c(C)c2cnc(Nc3ccc(N4CCC(N(C)CC(=O)O)CC4)cn3)nc2n(C2CCCC2)c1=O ; CC(=O)c1c(C)c2cnc(Nc3ccc(N4CCC(N(C)CC(=O)N5CCC(Nc6nc(NC(C)C)ccc6C(=O)Nc6cc(C)n(C)n6)CC5)CC4)cn3)nc2n(C2CCCC2)c1=O]

CPD-465 was synthesized following the standard procedure for preparing CPD-261 (7.0 mg, 37% yield) as a yellow solid. MS (ESI) m/z=887.8 [M+H]⁺.

### Example 522. 2-((1-(N-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)-N-methylglycyl)piperidin-4-yl)amino)-N-(2-cyclopropyl-1-methyl-1H-imidazol-4-yl)-6-(isopropylamino)nicotinamide (CPD-466)

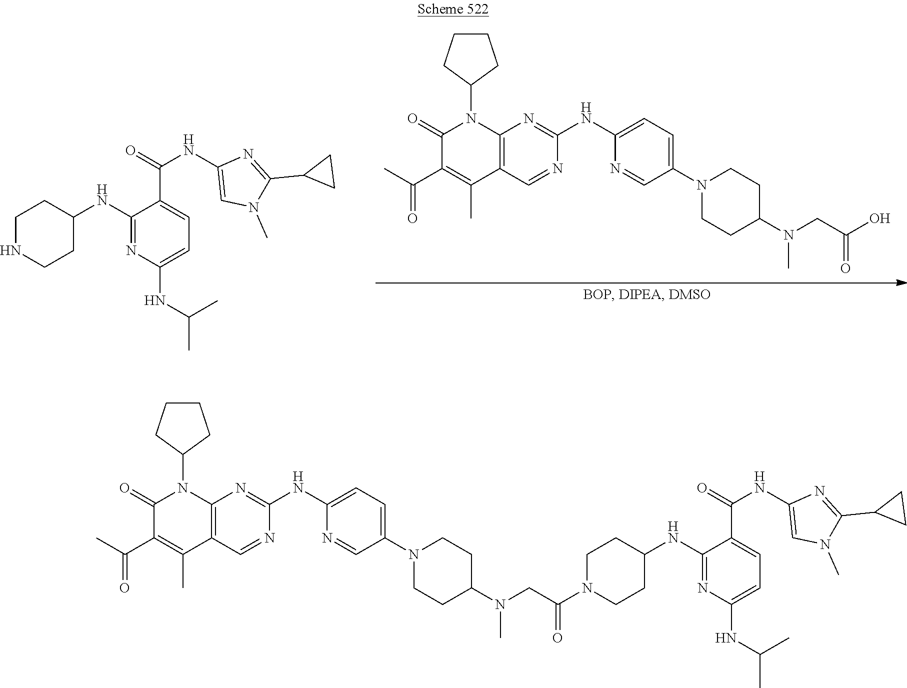 [SMILES: CC(C)Nc1ccc(C(=O)Nc2cn(C)c(C3CC3)n2)c(NC2CCNCC2)n1 ; CC(=O)c1c(C)c2cnc(Nc3ccc(N4CCC(N(C)CC(=O)O)CC4)cn3)nc2n(C2CCCC2)c1=O ; CC(=O)c1c(C)c2cnc(Nc3ccc(N4CCC(N(C)CC(=O)N5CCC(Nc6nc(NC(C)C)ccc6C(=O)Nc6cn(C)c(C7CC7)n6)CC5)CC4)cn3)nc2n(C2CCCC2)c1=O]

CPD-466 was synthesized following the standard procedure for preparing CPD-261 (6.0 mg, 31% yield) as a yellow solid. MS (ESI) m/z=913.8 [M+H]⁺.

### Example 523. 2-((6-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetamido)spiro[3.3]heptan-2-yl)amino)-N-(6-(dimethylamino)pyridazin-3-yl)-6-(isopropylamino)nicotinamide (CPD-467)

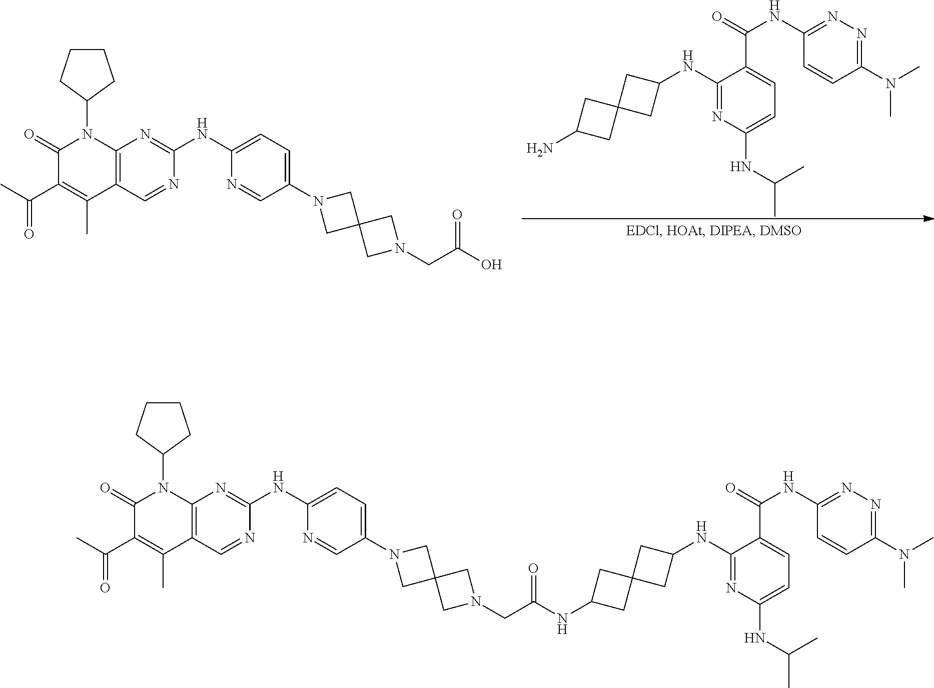 [SMILES: CC(=O)c1c(C)c2cnc(Nc3ccc(N4CC5(CN(CC(=O)O)C5)C4)cn3)nc2n(C2CCCC2)c1=O ; CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CC5(CN(CC(=O)NC6CC7(C6)CC(Nc6nc(NC(C)C)ccc6C(=O)Nc6ccc(N(C)C)nn6)C7)C5)C4)cn3)nc2n(C2CCCC2)c1=O]

CPD-467 was synthesized following the standard procedure for preparing CPD-143 (1.3 mg, 13% yield) as a yellow solid. MS (ESI) m/z=924.7 [M+H]⁺.

### Example 524. 2-((3-((2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetamido)methyl)cyclobutyl)amino)-N-(6-(dimethylamino)pyridazin-3-yl)-6-(isopropylamino)nicotinamide (CPD-468)

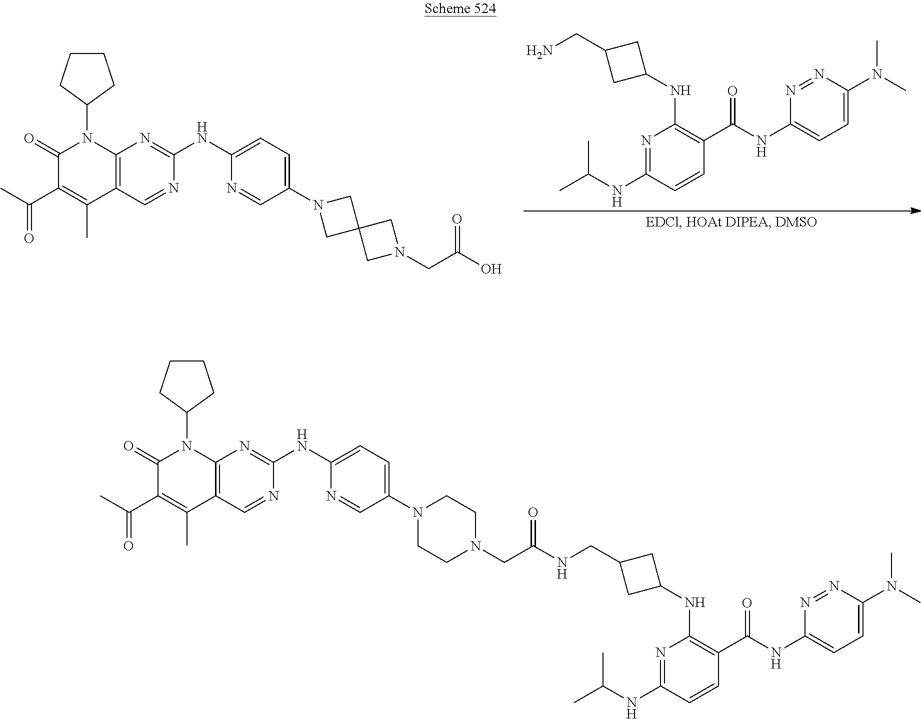 [SMILES: CC(=O)c1c(C)c2cnc(Nc3ccc(N4CC5(CN(CC(=O)O)C5)C4)cn3)nc2n(C2CCCC2)c1=O ; CC(C)Nc1ccc(C(=O)Nc2ccc(N(C)C)nn2)c(NC2CC(CN)C2)n1 ; CC(=O)c1c(C)c2cnc(Nc3ccc(N4CCN(CC(=O)NCC5CC(Nc6nc(NC(C)C)ccc6C(=O)Nc6ccc(N(C)C)nn6)C5)CC4)cn3)nc2n(C2CCCC2)c1=O]

CPD-468 was synthesized following the standard procedure for preparing CPD-143 (1.3 mg, 12% yield) as a yellow solid. MS (ESI) m/z=898.6 [M+H]⁺.

### Example 525. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)piperidin-4-yl)amino)-N-(6-(dimethylamino)pyridazin-3-yl)-6-(isopropylamino)nicotinamide (CPD-469)

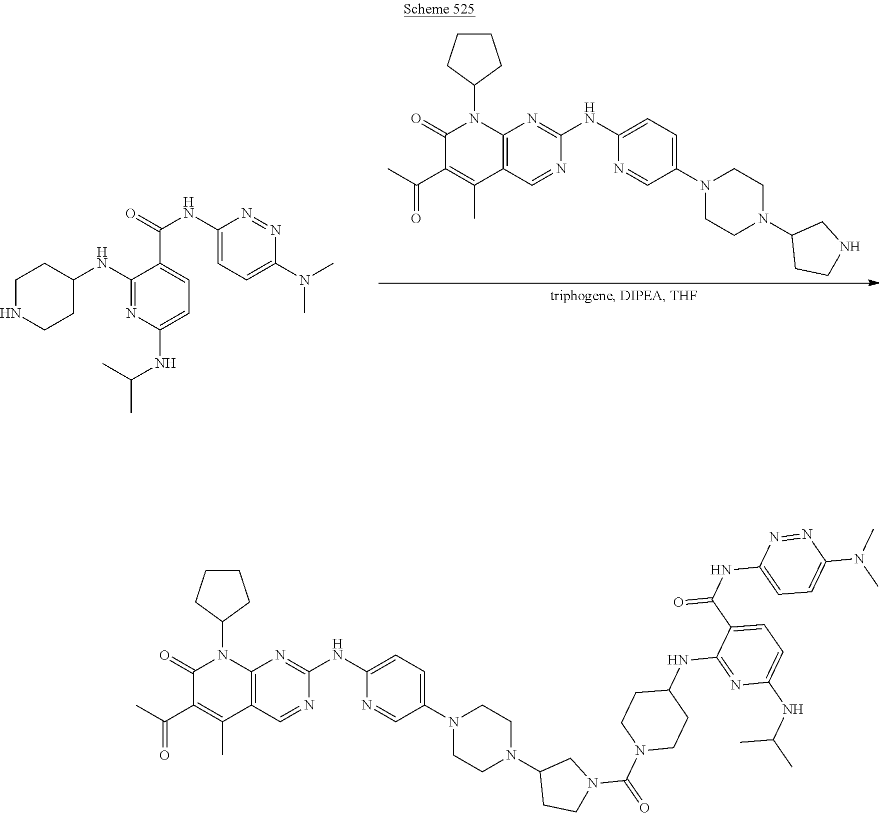 [SMILES: CC(C)Nc1ccc(C(=O)Nc2ccc(N(C)C)nn2)c(NC2CCNCC2)n1 ; CC(=O)c1c(C)c2cnc(Nc3ccc(N4CCN(C5CCNC5)CC4)cn3)nc2n(C2CCCC2)c1=O ; CC(=O)c1c(C)c2cnc(Nc3ccc(N4CCN(C5CCN(C(=O)N6CCC(Nc7nc(NC(C)C)ccc7C(=O)Nc7ccc(N(C)C)nn7)CC6)C5)CC4)cn3)nc2n(C2CCCC2)c1=O]

A mixture of 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (7 mg, 13.55 μmol) and DIPEA (6.99 mg, 54.2 μmol) in THF (4 mL) was added triphosgene (6.03 mg, 20.32 μmol) at 0° C. The mixture was stirred at the same temperature for 0.5 h. Then N-(6-(dimethylamino)pyridazin-3-yl)-6-(isopropylamino)-2-(piperidin-4-ylamino)nicotinamide (6.48 mg, 16.26 gmol) was added at 0° C. The reaction mixture stirred at rt for 16 h. The reaction mixture was poured into water (20 mL) and extracted with dichloromethane (3×20 mL). The combined organic layers were washed with saturated brine (2×15 mL), dried over anhydrous sodium sulfate, filtered, and evaporated under reduced pressure. The resulting residue was purified by prep-HPLC to give the desired product (0.9 mg, 6% yield) as a yellow solid. MS (ESI) m/z=942.0 [M+H]⁺.

### Example 526. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)spiro[3.3]heptan-2-yl)amino)-N-(6-(dimethylamino)pyridazin-3-yl)-6-(isopropylamino)nicotinamide (CPD-470)

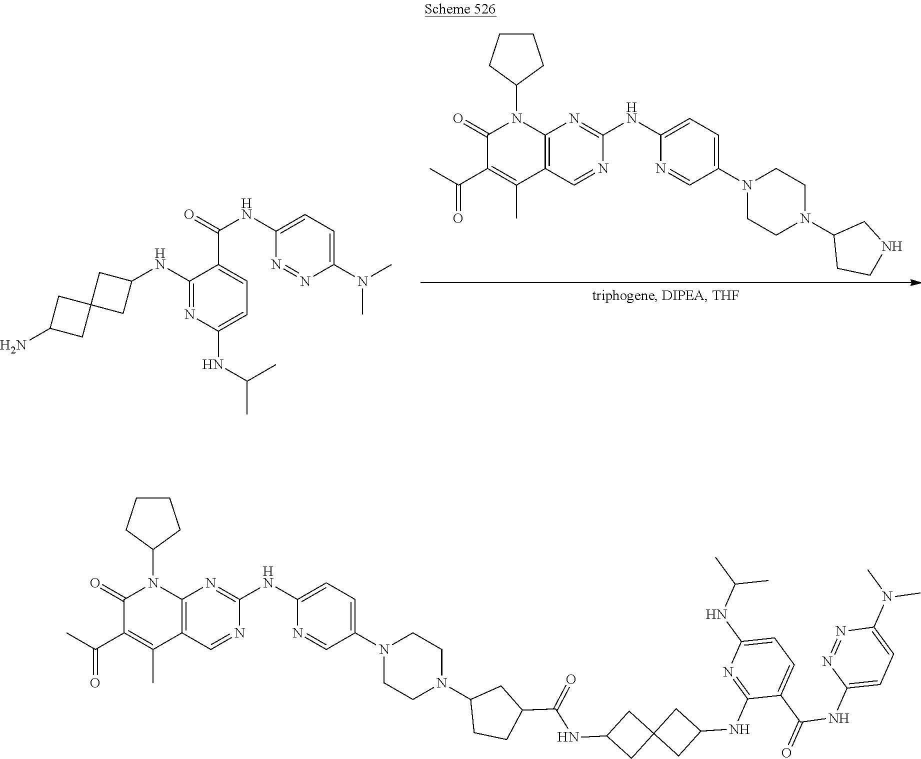 [SMILES: CC(C)Nc1ccc(C(=O)Nc2ccc(N(C)C)nn2)c(NC2CC3(CC(N)C3)C2)n1 ; CC(=O)c1c(C)c2cnc(Nc3ccc(N4CCN(C5CCNC5)CC4)cn3)nc2n(C2CCCC2)c1=O ; CC(=O)c1c(C)c2cnc(Nc3ccc(N4CCN(C5CCC(C(=O)NC6CC7(C6)CC(Nc6nc(NC(C)C)ccc6C(=O)Nc6ccc(N(C)C)nn6)C7)C5)CC4)cn3)nc2n(C2CCCC2)c1=O]

CPD-470 was synthesized following the standard procedure for preparing CPD-469 (1.2 mg, 10% yield) as a yellow solid. MS (ESI) m/z=968.0 [M+H]⁺.

### Example 527. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclobutyl)amino)-N-(6-(dimethylamino)pyridazin-3-yl)-6-((methyl-d3)amino)nicotinamide (CPD-471)

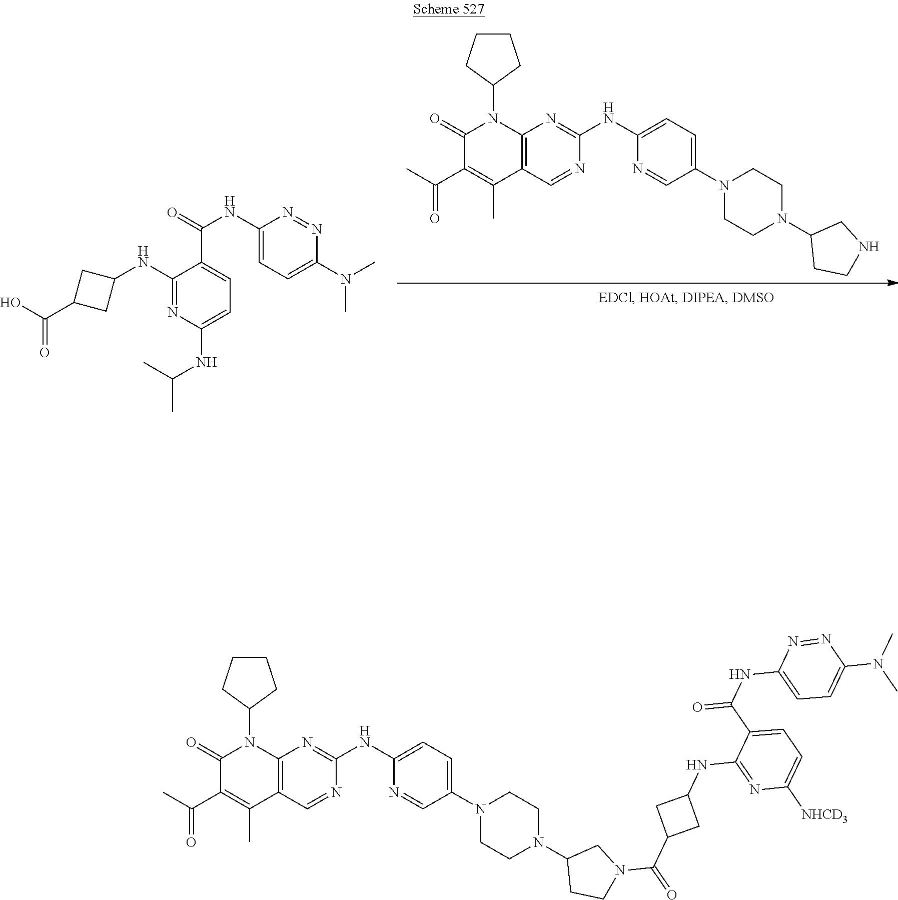

CPD-471 was synthesized following the standard procedure for preparing CPD-143 (1.6 mg, 7% yield) as a yellow solid. MS (ESI) m/z=888.1 [M+H]⁺.

### Example 528. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-(isopropylamino)nicotinamide (CPD-472)

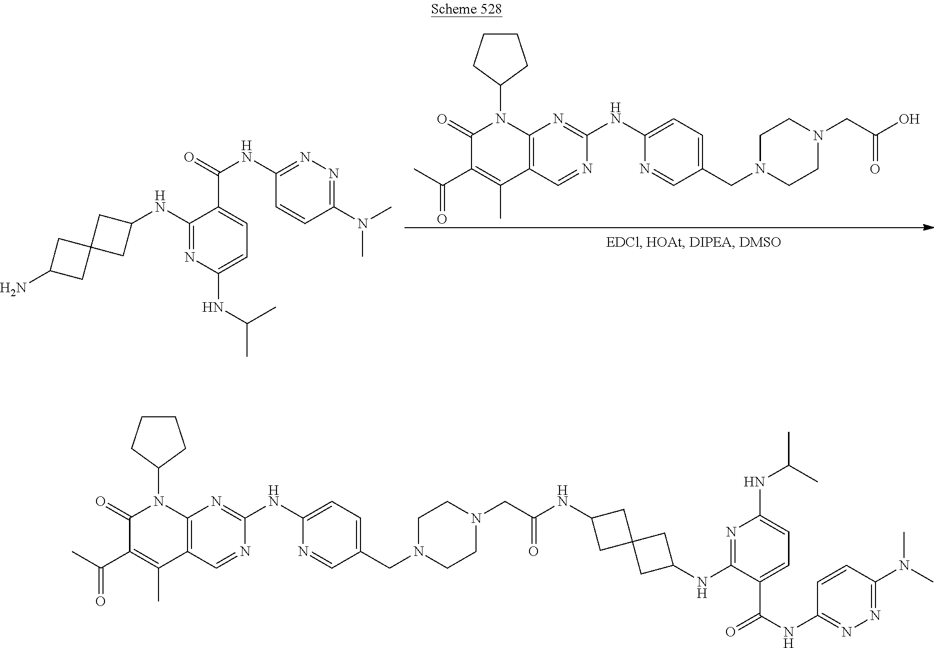

CPD-472 was synthesized following the standard procedure for preparing CPD-143 (3.9 mg, 24% yield) as a yellow solid. MS (ESI) m/z=927.1 [M+H]⁺.

### Example 529. 2-((4-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclohexyl)amino)-N-(6-(dimethylamino)pyridazin-3-yl)-6-(isopropylamino)nicotinamide (CPD-473)

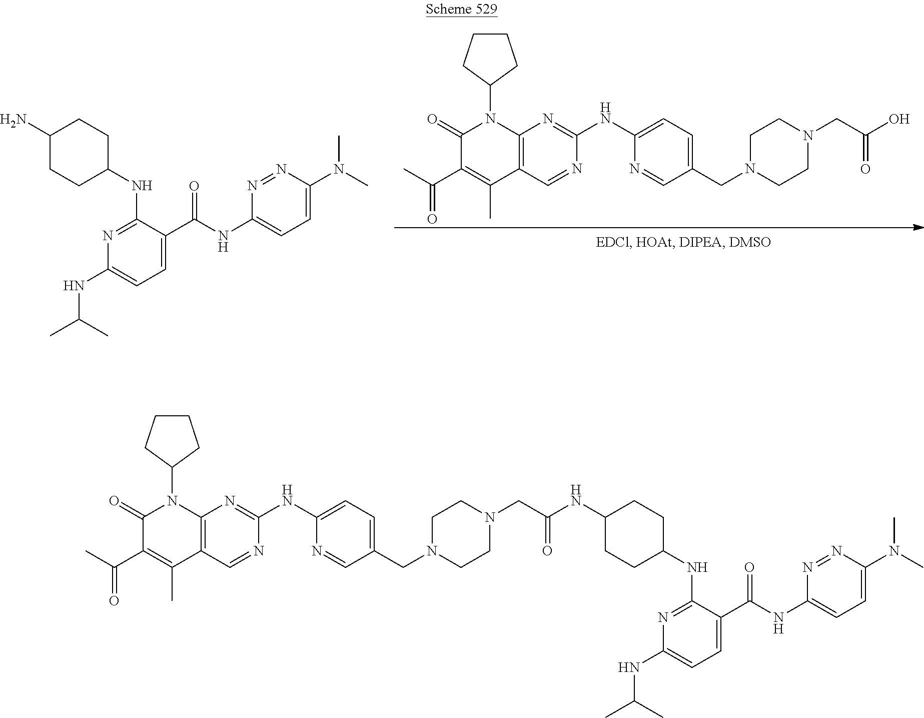

CPD-473 was synthesized following the standard procedure for preparing CPD-143 (1.1 mg, 8% yield) as a yellow solid. MS (ESI) m/z=915.1 [M+H]⁺.

### Example 530. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6-(dimethylamino)pyridazin-3-yl)-6-((methyl-d3)amino)nicotinamide (CPD-474)

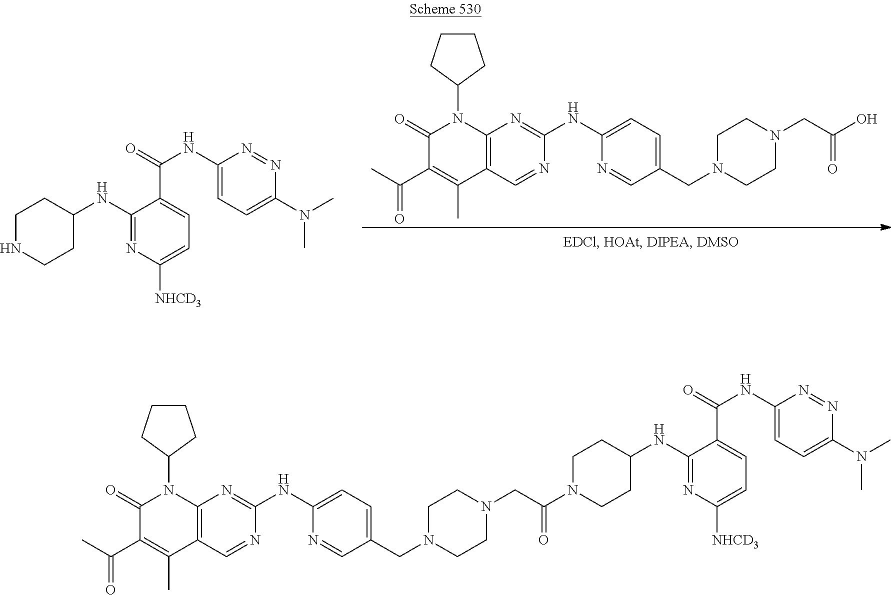

CPD-474 was synthesized following the standard procedure for preparing CPD-143 (3.5 mg, 42% yield) as a yellow solid. MS (ESI) m/z=875.8 [M+H]⁺.

### Example 531. 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclobutyl)amino)-N-(6-(dimethylamino)pyridazin-3-yl)-6-(isopropylamino)nicotinamide (CPD-475)

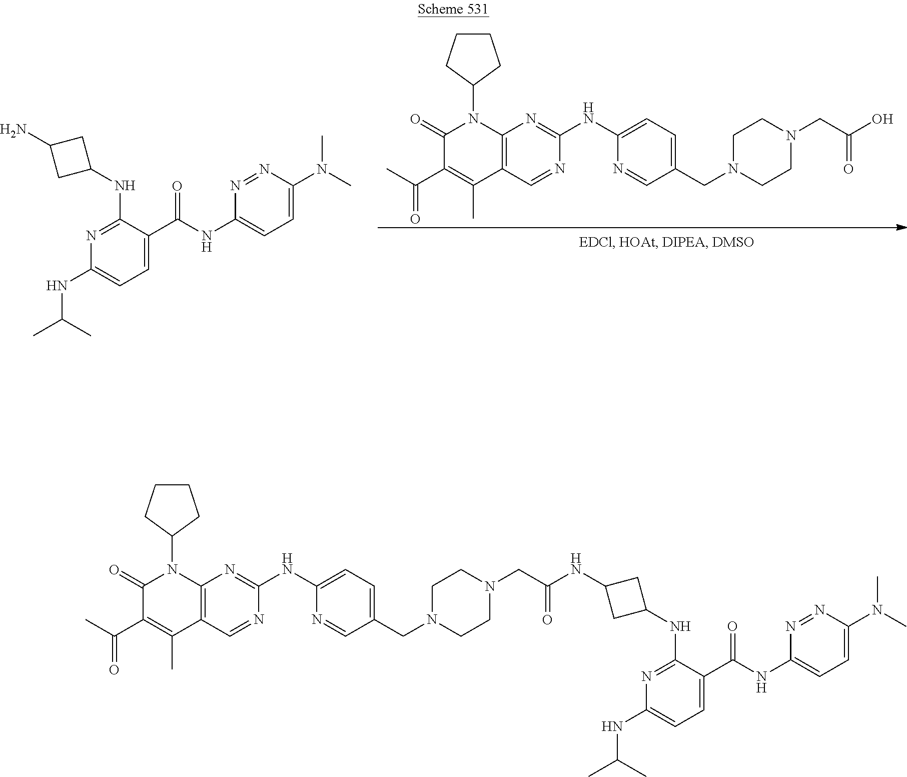

CPD-475 was synthesized following the standard procedure for preparing CPD-143 (3.5 mg, 41% yield) as a yellow solid. MS (ESI) m/z=886.7 [M+H]⁺.

### Example 532. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-((methyl-d3)amino)nicotinamide (CPD-476)

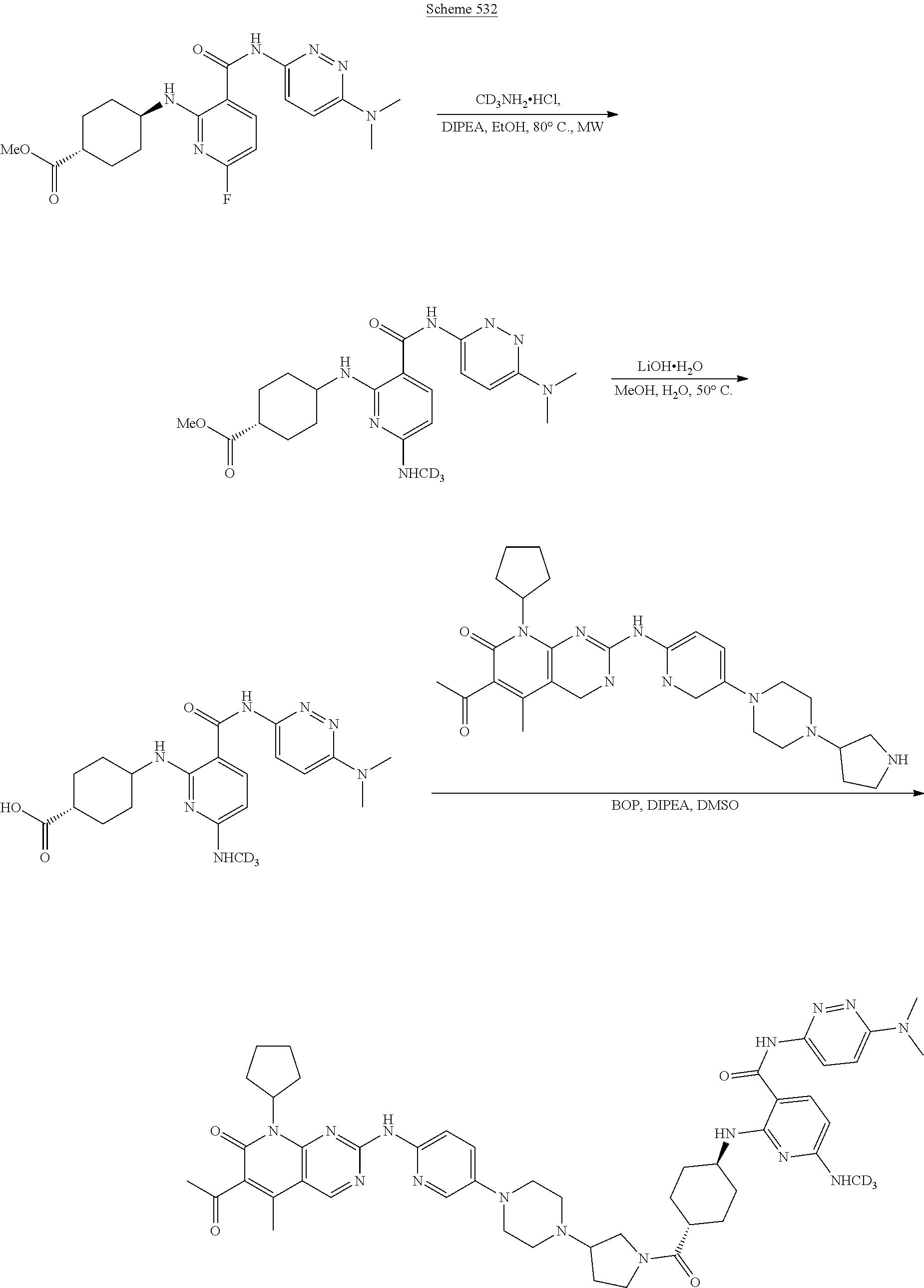

CPD-476 was synthesized following the standard procedures for preparing CPD-82 (8.0 mg, 21% yield over 3 steps) as a yellow solid. MS (ESI) m/z=915.8 [M+H]⁺.

### Example 533. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-((methyl-d₃)amino)nicotinamide (CPD-477)

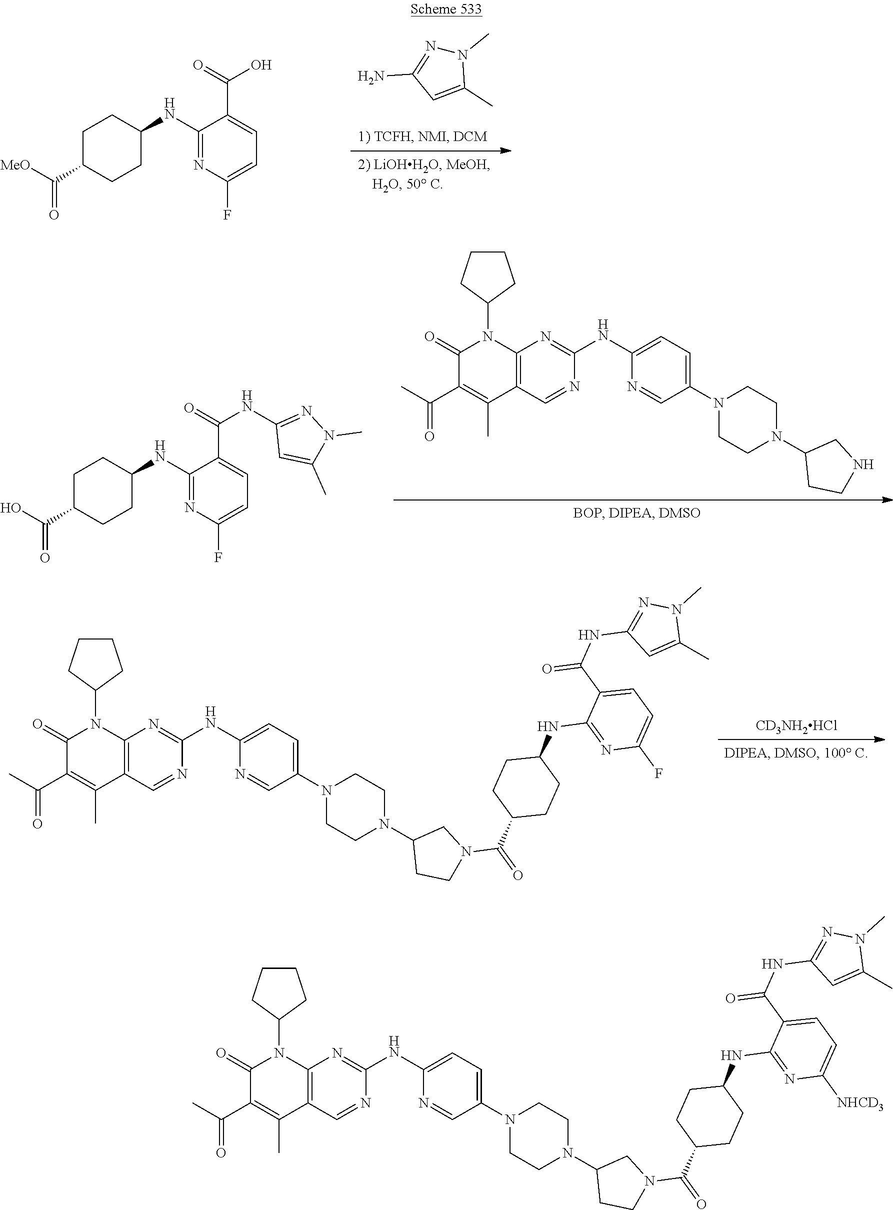

### Step 1. Synthesis of methyl trans-4-((3-((1,5-dimethyl-1H-pyrazol-3-yl)carbamoyl)-6-fluoropyridin-2-yl)amino)cyclohexane-1-carboxylate

To a solution of 6-fluoro-2-((trans-4-(methoxycarbonyl)cyclohexyl)amino)nicotinic acid (120 mg, 0.40 mmol) in dichloromethane (5 mL) were added 1,5-dimethyl-1H-pyrazol-3-amine (45 mg, 0.40 mmol), NMI (166 mg, 2.03 mmol) and TCFH (227 mg, 0.81 mmol). The reaction mixture was stirred at rt for 3 h. The mixture was concentrated and purified by silica gel chromatography to give the desired product (100 mg, 63% yield) as a light-yellow solid. MS(ESI) m/z=390.5 [M+H]⁺.

### Step 2. Synthesis of trans-4-((3-((6-(dimethylamino)pyridazin-3-yl)carbamoyl)-6-fluoropyridin-2-yl)amino)cyclohexane-1-carboxylic acid

To a solution of methyl trans-4-((3-((1,5-dimethyl-1H-pyrazol-3-yl)carbamoyl)-6-fluoropyridin-2-yl)amino)cyclohexane-1-carboxylate (100 mg, 0.25 mmol) in MeOH (10 mL) and water (3 mL) was added LiOH H₂O (108 mg, 2.57 mmol). The reaction mixture was stirred at 50° C. for 6 h. The mixture was concentrated. The residue was re-dissolved in H₂O (20 mL). The pH value was adjusted to 4 by adding aq. HCl (1 N). The mixture was extracted with ethyl acetate (3×20 mL). The combined organic extracts were washed with water (20 mL), dried with Na₂SO₄, filtered and concentrated. The resulting residue was purified by reverse-phase chromatography to provide the desired product (65 mg, 67% yield) as a light-yellow solid. MS (ESI) m/z=376.34 [M+H]⁺.

### Step 3. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-fluoronicotinamide

To a solution of 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (65 mg, 0.12 mmol) in DMSO (2 mL) were added trans-4-((3-((6-(dimethylamino)pyridazin-3-yl)carbamoyl)-6-fluoropyridin-2-yl)amino)cyclohexane-1-carboxylic acid (57 mg, 0.15 mmol), DIPEA (81 mg, 0.63 mmol) and BOP (111 mg, 0.25 mmol). The reaction mixture was stirred at rt for 2 h. The mixture was purified by prep-HPLC to give the desired product (50 mg, 45% yield) as a yellow solid. MS(ESI) m/z=874.7 [M+H]⁺.

### Step 4. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-((methyl-d₃)amino)nicotinamide

To a solution of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-fluoronicotinamide (25 mg, 0.028 mmol) in DMSO (2 mL) were added DIPEA (37 mg, 0.28 mmol) and CD₃NH₂—HCl (20 mg, 0.28 mmol). The mixture was stirred at 100° C. for 3 h. After cooling down to rt, the mixture was purified by reverse-phase chromatography to give the desired product (8 mg, 31% yield) as a yellow solid. MS(ESI) m/z=888.7 [M+H]⁺.

### Example 534. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(1,5-dimethyl-1H-pyrazol-3-yl)-6-(isopropylamino)nicotinamide (CPD-478)

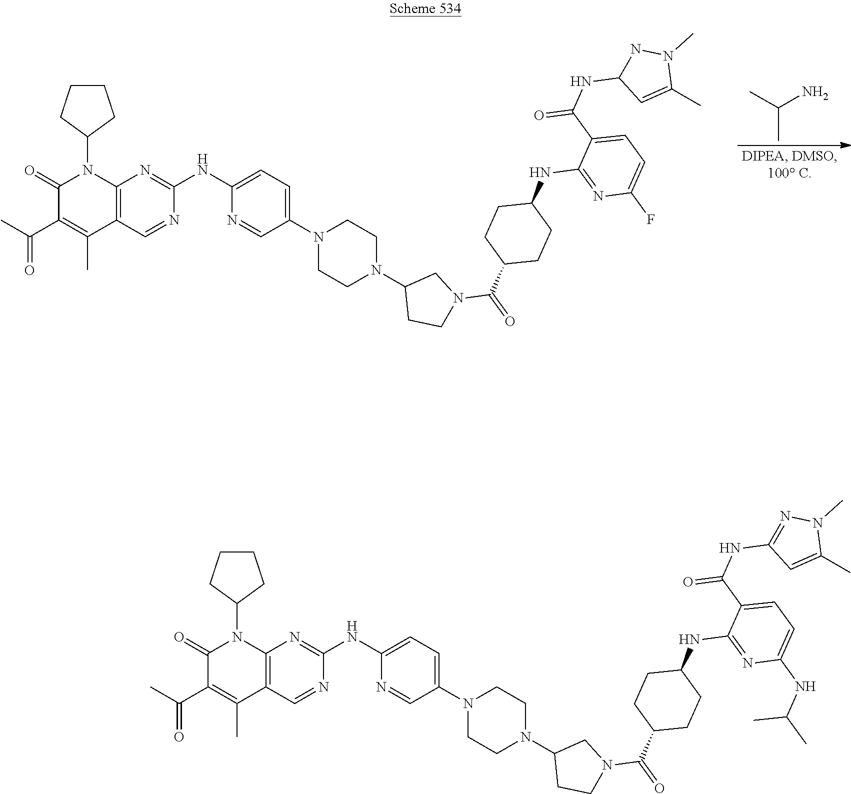

CPD-478 was synthesized following the standard procedure for preparing CPD-477 (6.0 mg, 23% yield) as a yellow solid. MS (ESI) m/z=913.8 [M+H]⁺.

### Example 535. 2-((3-((2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)methyl)bicyclo[1.1.1]pentan-1-yl)amino)-N-(6-(dimethylamino)pyridazin-3-yl)-6-(isopropylamino)nicotinamide (CPD-479)

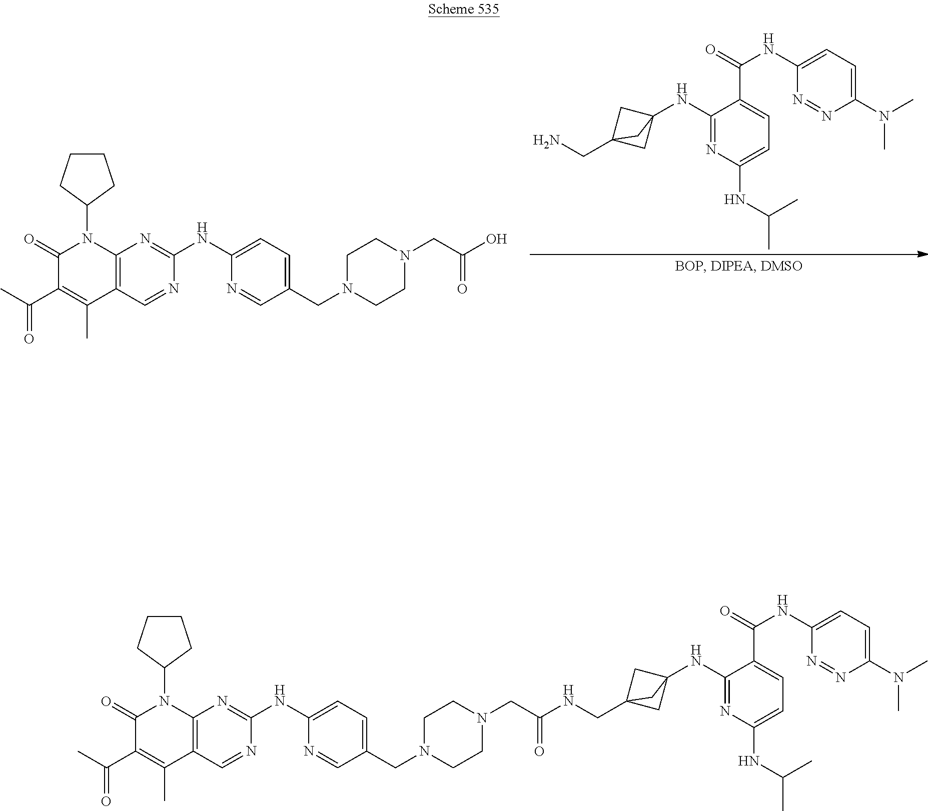 [SMILES: CC(=O)c1c(C)c2cnc(Nc3ccc(CN4CCN(CC(=O)O)CC4)cn3)nc2n(C2CCCC2)c1=O ; CC(C)Nc1ccc(C(=O)Nc2ccc(N(C)C)nn2)c(NC23CC(CN)(C2)C3)n1 ; CC(=O)c1c(C)c2cnc(Nc3ccc(CN4CCN(CC(=O)NCC56CC(Nc7nc(NC(C)C)ccc7C(=O)Nc7ccc(N(C)C)nn7)(C5)C6)CC4)cn3)nc2n(C2CCCC2)c1=O]

CPD-479 was synthesized following the standard procedure for preparing CPD-261 (8.0 mg, 30% yield) as a yellow solid. MS (ESI) m/z=912.7 [M+H]⁺.

### Example 536. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(2-cyclopropyl-1-methyl-1H-imidazol-4-yl)-6-((methyl-d₃)amino)nicotinamide (CPD-480)

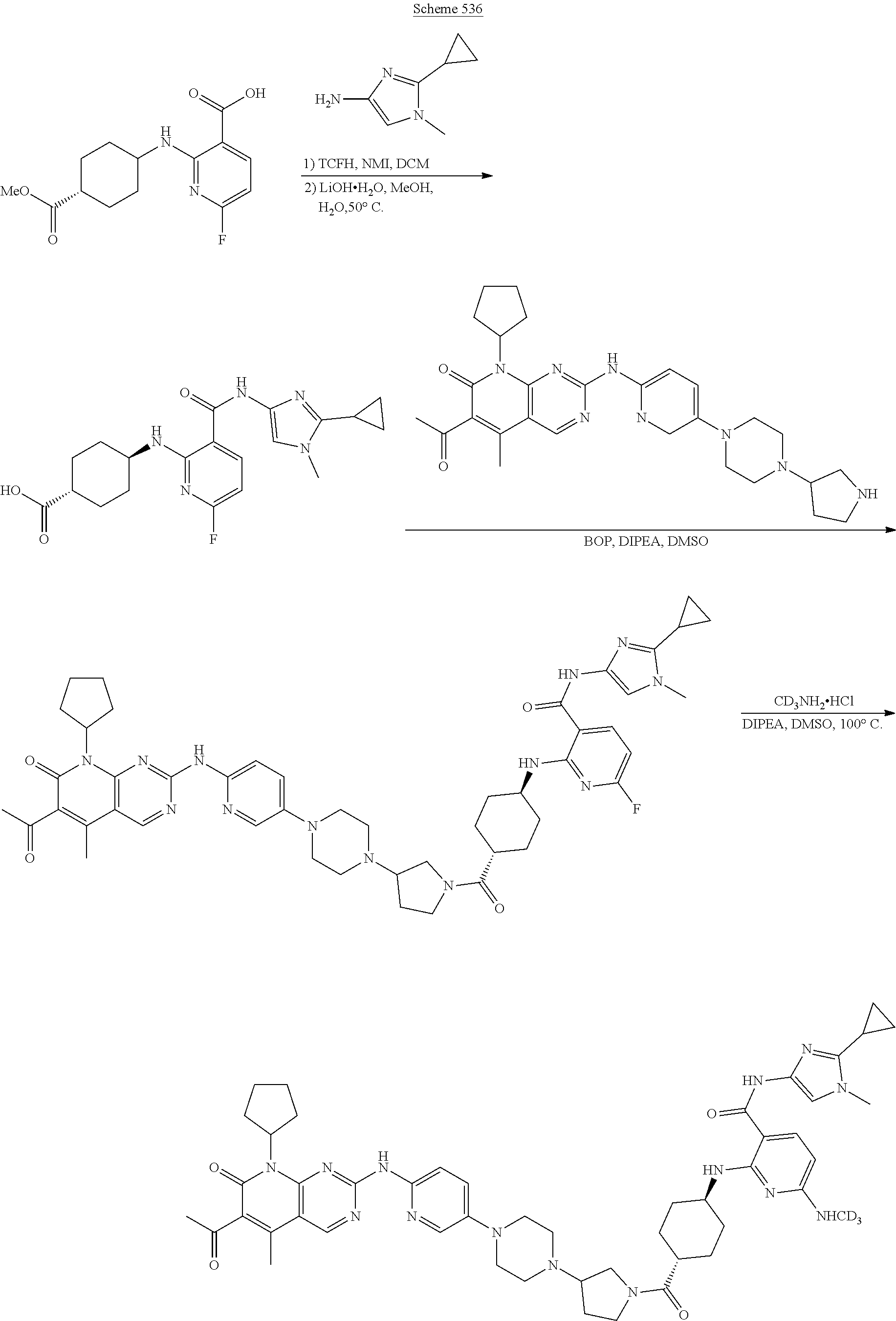

CPD-480 was synthesized following the standard procedures for preparing CPD-477 (2.0 mg, 1% yield over 4 steps) as a yellow solid. MS (ESI) m/z=914.9 [M+H]⁺.

### Example 537. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(2-cyclopropyl-1-methyl-1H-imidazol-4-yl)-6-(isopropylamino)nicotinamide (CPD-481)

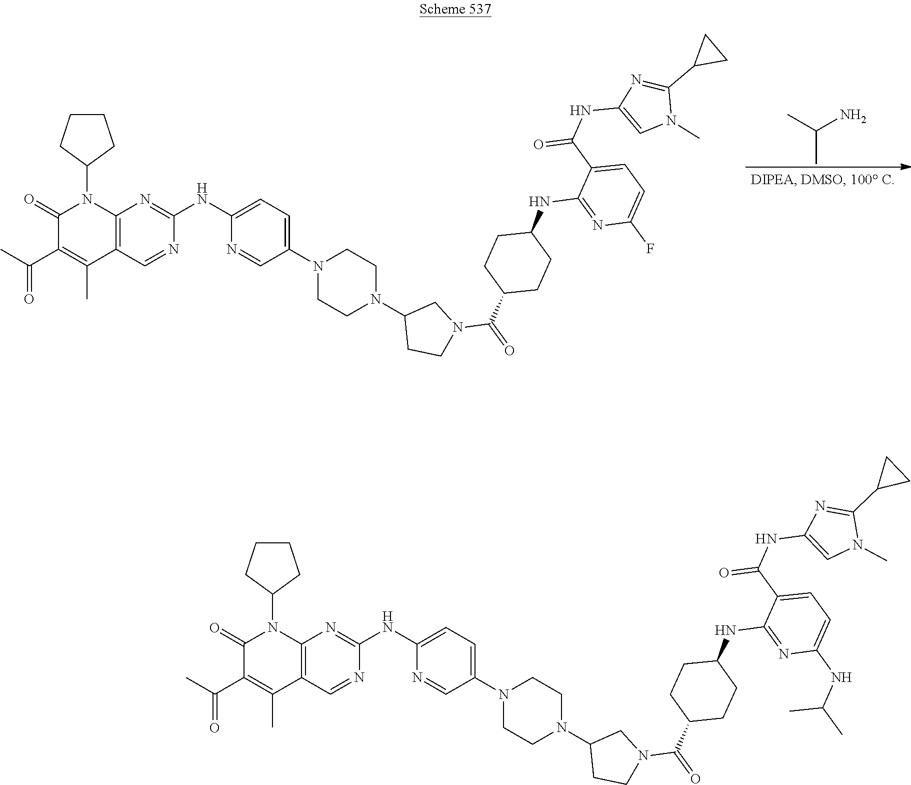

CPD-481 was synthesized following the standard procedure for preparing CPD-477 (3.0 mg, 19% yield) as a yellow solid. MS (ESI) m/z=939.8 [M+H]⁺.

### Example 538. 2-((1-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)piperidin-4-yl)amino)-N-(6-(dimethylamino)pyridazin-3-yl)-6-((methyl-d₃)amino)nicotinamide (CPD-482)

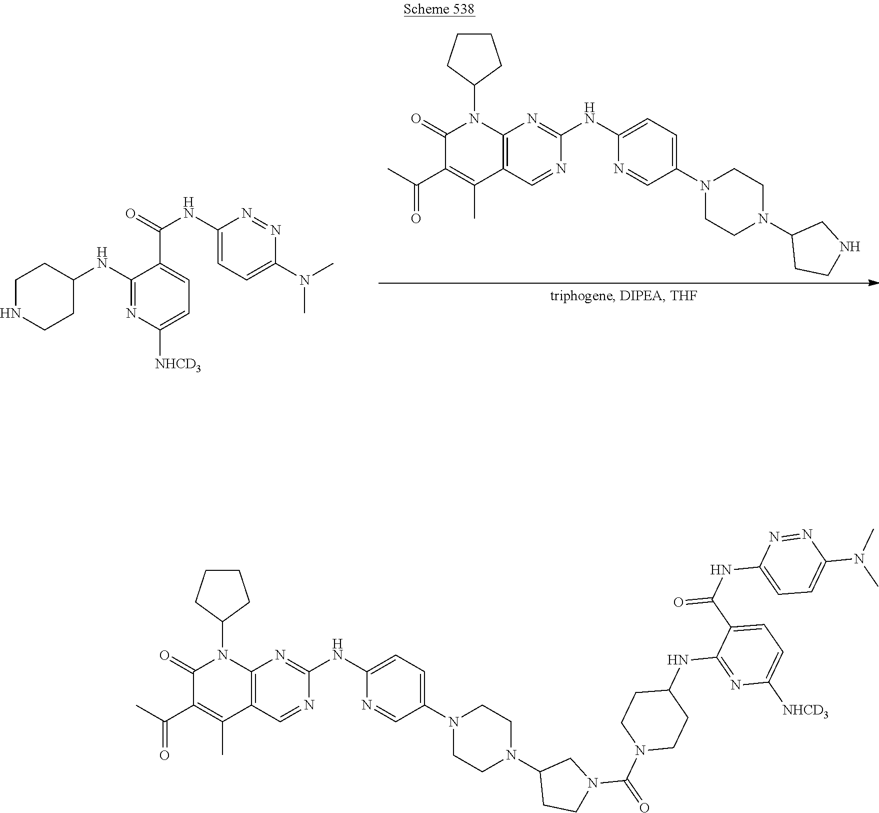

CPD-482 was synthesized following the standard procedure for preparing CPD-469 (0.5 mg, 2% yield) as a yellow solid. MS (ESI) m/z=917.1 [M+H]⁺.

### Example 539. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)cyclohexyl)amino)-N-(6-(dimethylamino)pyridazin-3-yl)-6-(isopropylamino)nicotinamide (CPD-483)

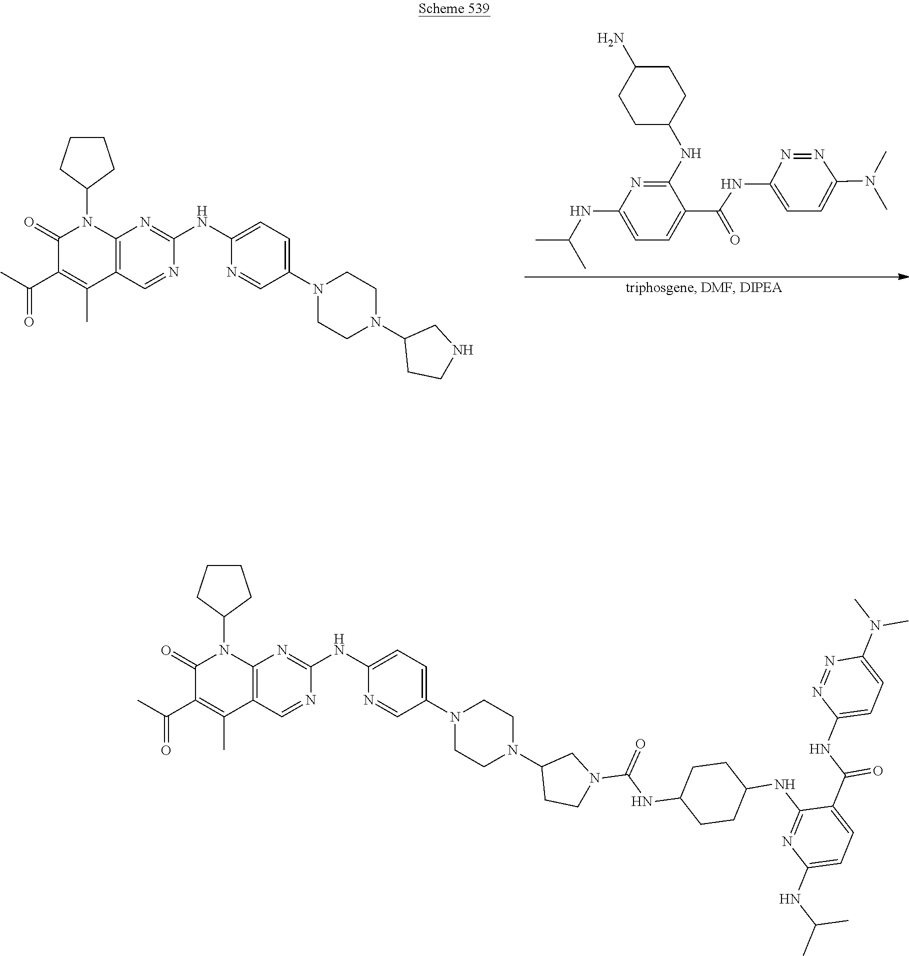 [SMILES: CC(=O)c1c(C)c2cnc(Nc3ccc(N4CCN(C5CCNC5)CC4)cn3)nc2n(C2CCCC2)c1=O ; CC(C)Nc1ccc(C(=O)Nc2ccc(N(C)C)nn2)c(NC2CCC(N)CC2)n1 ; CC(=O)c1c(C)c2cnc(Nc3ccc(N4CCN(C5CCN(C(=O)NC6CCC(Nc7nc(NC(C)C)ccc7C(=O)Nc7ccc(N(C)C)nn7)CC6)C5)CC4)cn3)nc2n(C2CCCC2)c1=O]

CPD-483 was synthesized following the standard procedure for preparing CPD-469 (1.1 mg, 7% yield) as a yellow solid. MS (ESI) m/z=956.1 [M+H]⁺.

### Example 540. 2-((1-(N-(2-(6-((6-acetyl-8-cyclopentyl-5-methyl-7-oxo-7,8-dihydropyrido[2,3-d]pyrimidin-2-yl)amino)pyridin-3-yl)-2-azaspiro[3.3]heptan-6-yl)-N-methylglycyl)piperidin-4-yl)amino)-N-(6-(dimethylamino)pyridazin-3-yl)-6-(isopropylamino)nicotinamide (CPD-484)

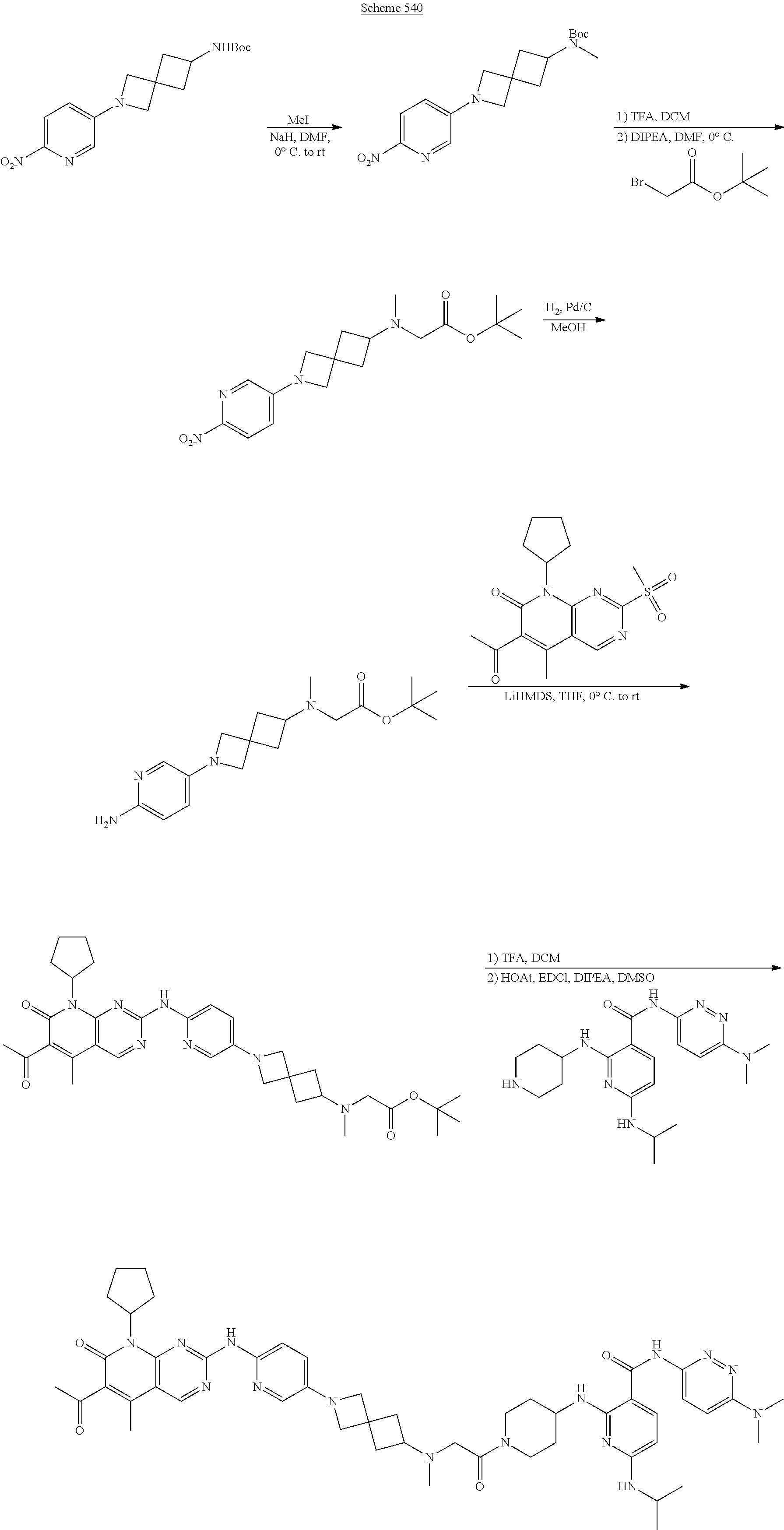

CPD-484 was synthesized following the standard procedures for preparing CPD-418 (3.5 mg, 6% yield over 7 steps) as a yellow solid. MS (ESI) m/z=926.8 [M+H]⁺.

### Example 541. 2-((3-((2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)methyl)bicyclo[1.1.1]pentan-1-yl)amino)-N-(6-(dimethylamino)pyridazin-3-yl)-6-(isopropylamino)nicotinamide (CPD-485)

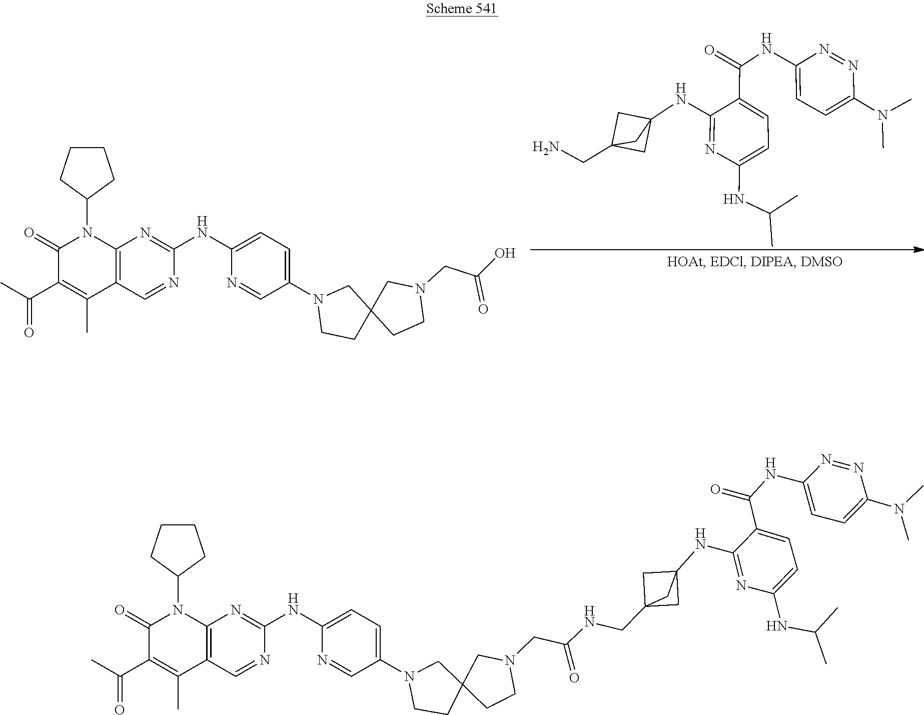 [SMILES: CC(=O)c1c(C)c2cnc(Nc3ccc(N4CCC5(CCN(CC(=O)O)C5)C4)cn3)nc2n(C2CCCC2)c1=O ; CC(C)Nc1ccc(C(=O)Nc2ccc(N(C)C)nn2)c(NC23CC(CN)(C2)C3)n1 ; CC(=O)c1c(C)c2cnc(Nc3ccc(N4CCC5(CCN(CC(=O)NCC67CC(Nc8nc(NC(C)C)ccc8C(=O)Nc8ccc(N(C)C)nn8)(C6)C7)C5)C4)cn3)nc2n(C2CCCC2)c1=O]

CPD-485 was synthesized following the standard procedure for preparing CPD-143 (0.7 mg, 9% yield) as a yellow solid. MS (ESI) m/z=938.8 [M+H]⁺.

### Example 542. 2-((6-((1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)carbamoyl)spiro[3.3]heptan-2-yl)amino)-N-(6-(dimethylamino)pyridazin-3-yl)-6-(isopropylamino)nicotinamide (CPD-486)

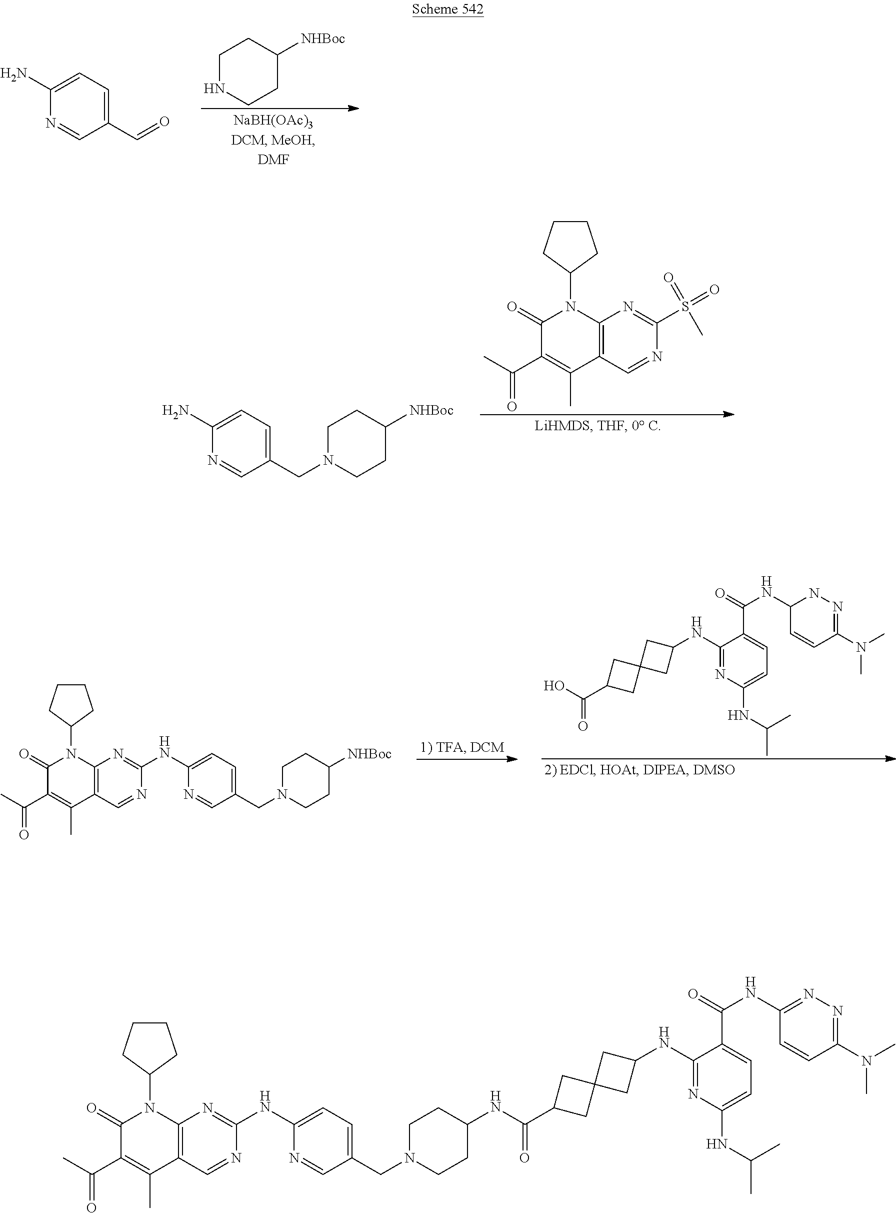

### Step 1. Synthesis of tert-butyl (1-((6-aminopyridin-3-yl)methyl)piperidin-4-yl) carbamate

A mixture of 6-aminopyridine-3-carbaldehyde (120 mg, 0.98 mmol) and tert-butyl piperidin-4-ylcarbamate (394 mg, 1.97 mmol) in dichloromethane (5 mL), MeOH (1 mL) and DMF (0.5 mL) was stirred at rt for 2 h, followed by adding NaBH(OAc)₃ (622 mg, 2.95 mmol) in portions at 0° C. The reaction mixture was stirred at rt for 12 h. The mixture was quenched with aq. NaHCO₃ solution (8 mL, 5 N) and extracted with dichloromethane (3×10 mL). The combined organic phase was washed with brine, dried over Na₂SO₄, filtered, and concentrated to give the crude. The residue was purified by silica gel chromatography to provide the desired product (120 mg, 40% yield) as a white solid. MS (ESI) m/z=307.3 [M+H]⁺.

The remaining steps were performed according to the procedures for preparing CPD-418 to provide the desired product (3.6 mg, 8% yield over 3 steps) as a yellow solid. MS (ESI) m/z=911.8 [M+H]⁺.

### Example 543. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-(isopropylamino)nicotinamide (CPD-487)

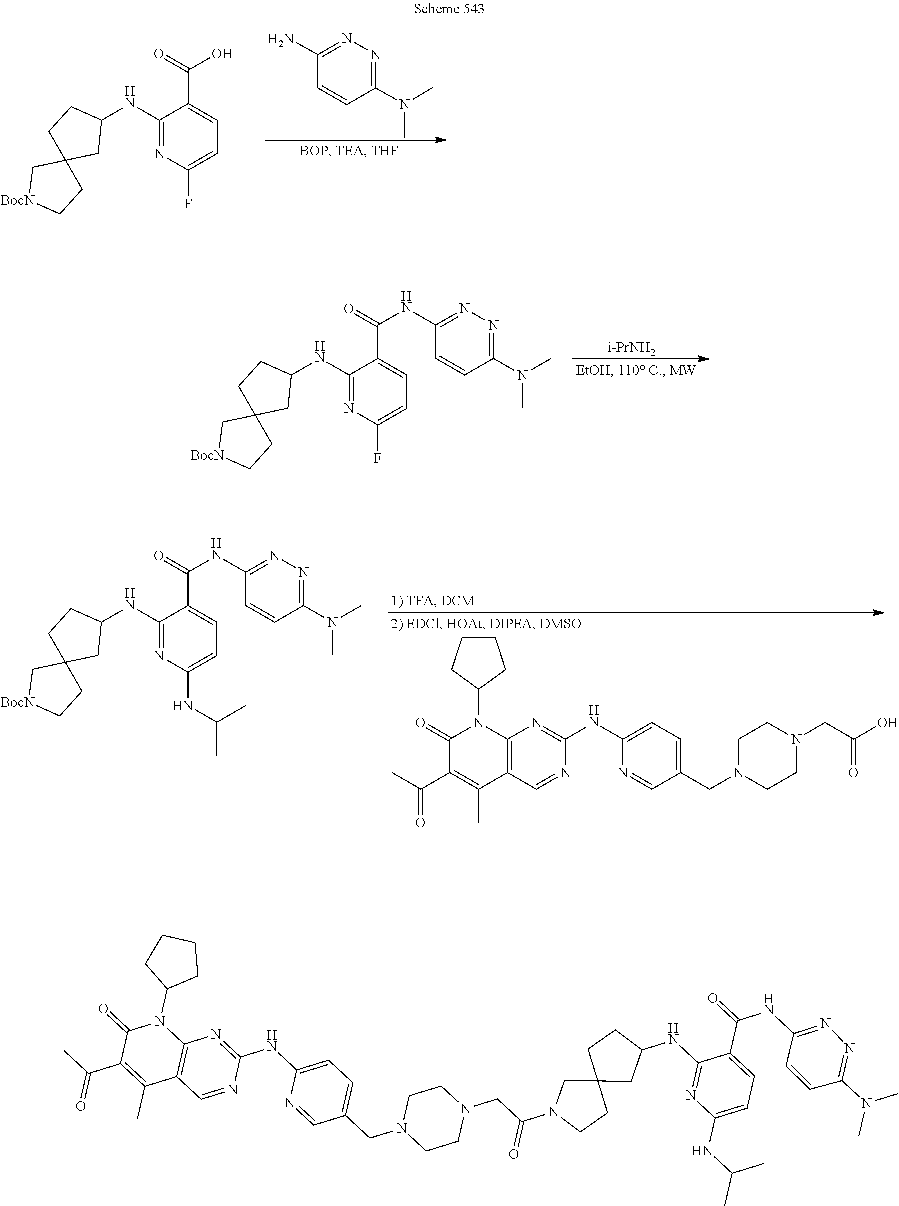

CPD-487 was synthesized following the standard procedures for preparing CPD-145 (12.0 mg, 16% yield over 4 steps) as a yellow solid. MS (ESI) m/z=940.8 [M+H]⁺.

### Example 544. 2-((2-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetyl)-2-azaspiro[3.3]heptan-6-yl)amino)-N-(6-(dimethylamino)pyridazin-3-yl)-6-((methyl-d₃)amino)nicotinamide (CPD-488)

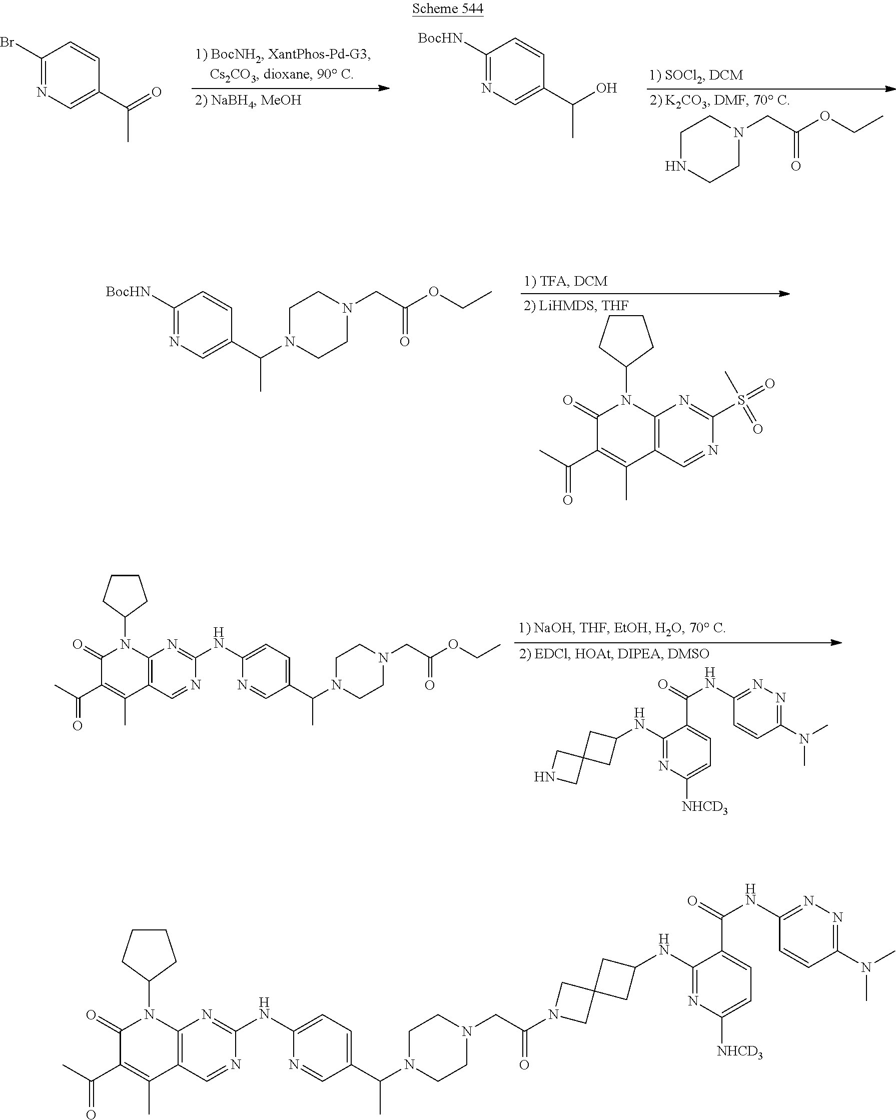

To a mixture of 1-(6-bromopyridin-3-yl)ethan-1-one (1 g, 5 mmol) and tert-butyl carbamate (1.2 g, 10 mmol) in dioxane (20 mL) were added Cs₂CO₃ (4.9 g, 15 mmol) and XantPhos-Pd-G3 (480 mg, 0.5 mmol) at room temperature. The reaction mixture was stirred at 90° C. for 16 h under N₂. The reaction mixture was poured into water (20 mL) and extracted with ethyl acetate (3×20 mL). The combined organic layers were washed with saturated brine (30 mL), dried over anhydrous Na₂SO₄, filtered and concentrated. The resulting residue was purified by silica gel flash chromatography to give the desired product (650 mg, 55%0 yield) as a yellow solid.

### Step 2. Synthesis of tert-butyl (5-(1-hydroxyethyl)pyridin-2-yl)carbamate

To a solution of tert-butyl (5-acetylpyridin-2-yl)carbamate (650 mg, 2.75 mmol) in MeOH (10 mL) was added NaBH₄ (210 mg, 5.5 mmol). The reaction mixture was stirred at rt for 1 h. The reaction mixture was poured into water (20 mL) and extracted with ethyl acetate (3×20 mL). The combined organic layers were washed with saturated brine (30 mL), dried over anhydrous Na₂SO₄, filtered and concentrated. The resulting residue was purified by silica gel flash chromatography to give the desired product (510 mg, 77% yield) as a yellow solid.

### Step 3. Synthesis of tert-butyl (5-(1-chloroethyl)pyridin-2-yl)carbamate

To a solution of tert-butyl (5-(1-hydroxyethyl)pyridin-2-yl)carbamate (410 mg, 1.72 mmol) in dichloromethane (10 mL) was added thionyl chloride (2.1 g, 17 mmol). The mixture was stirred at rt for 1 h. The mixture was concentrated to give the crude product (441 mg, 99% yield) as a brown solid. This compound was used directly in the next step without further purification.

### Step 4. Synthesis of ethyl 2-(4-(1-(6-((tert-butoxycarbonyl)amino)pyridin-3-yl)ethyl)piperazin-1-yl)acetate

To a mixture of tert-butyl (5-(1-chloroethyl)pyridin-2-yl)carbamate (441 mg, 1.72 mmol) and ethyl 2-(piperazin-1-yl)acetate (296 mg, 1.72 mmol) in DMF (10 mL) was added K₂CO₃ (712 mg, 5.2 mmol) at room temperature. The reaction mixture was stirred at 70° C. for 16 h. The reaction mixture was poured into water (20 mL) and extracted with ethyl acetate (3×20 mL). The combined organic layers were washed with saturated brine (20 mL), dried over anhydrous Na₂SO₄, filtered and concentrated. The resulting residue was purified by reverse-phase chromatography to give the desired product (123 mg, 18% yield) as a white solid. MS (ESI) m/z=393.4 [M+H]⁺.

### Step 5. Synthesis of ethyl 2-(4-(1-(6-aminopyridin-3-yl)ethyl)piperazin-1-yl)acetate

To a solution of ethyl 2-(4-(1-(6-((tert-butoxycarbonyl)amino)pyridin-3-yl)ethyl)piperazin-1-yl)acetate (123 mg, 0.3 mmol) in dichloromethane (2 mL) was added TFA (1 mL). The reaction mixture was stirred at rt for 1 h. The mixture was concentrated to give the desired product (123 mg, 96% yield) as a brown solid. This compound was used directly in the next step without further purification. MS (ESI) m/z=293.4 [M+H]⁺.

### Step 6. Synthesis of ethyl 2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetate

To a mixture of ethyl 2-(4-(1-(6-aminopyridin-3-yl)ethyl)piperazin-1-yl)acetate (123 mg, 0.3 mmol) and 6-acetyl-8-cyclopentyl-5-methyl-2-(methylsulfonyl)pyrido[2,3-d]pyrimidin-7(8H)-one (85 mg, 0.2 mmol) in THF (5 mL) was added LiHMDS (0.6 mL, 1 M in THF) at 0° C. The reaction mixture was stirred at 0° C. for 1 h. The reaction mixture was quenched with water (10 mL) and extracted with ethyl acetate (3×20 mL). The combined organic layers were washed with saturated brine (20 mL), dried over anhydrous Na₂SO₄, filtered and concentrated. The resulting residue was purified by silica gel flash chromatography to give the desired product (35 mg, 21% yield) as a yellow oil. MS (ESI) m/z=562.9 [M+H]⁺.

### Step 7. Synthesis of 2-(4-(I-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetic acid

A mixture of ethyl 2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetate (35 mg, 0.06 mmol) and NaOH (25 mg, 0.6 mmol) in THF (2 mL), EtOH (1 mL) and H₂O (1 mL) was stirred at 70° C. for 2 h. The resulting mixture was purified by reverse-phase chromatography to give the desired product (36 mg, 89% yield) as a white solid. MS (ESI) m/z=534.9 [M+H]⁺.

### Step 8. Synthesis of 2-((2-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetyl)-2-azaspiro[3.3]heptan-6-yl)amino)-N-(6-(dimethylamino)pyridazin-3-yl)-6-((methyl-d₃)amino)nicotinamide

A mixture of 2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetic acid (10 mg, 0.02 mmol), 2-((2-azaspiro[3.3]heptan-6-yl)amino)-N-(6-(dimethylamino)pyridazin-3-yl)-6-((methyl-d3)amino)nicotinamide (7.2 mg, 0.02 mmol), EDCI (5.5 mg, 0.03 mmol), HOAt (3.9 mg, 0.03 mmol) and DIPEA (12 mg, 0.1 mmol) in DMSO (2 mL) was stirred at rt for 16 h. The reaction mixture was purified by reverse-phase chromatography to give the desired product (7.5 mg, 44% yield) as a yellow solid. MS (ESI) m/z=902.1 [M+H]⁺.

### Example 545. 2-((2-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)-2-azaspiro[3.3]heptan-6-yl)amino)-N-(6-(dimethylamino)pyridazin-3-yl)-6-((methyl-d₃)amino)nicotinamide (CPD-489)

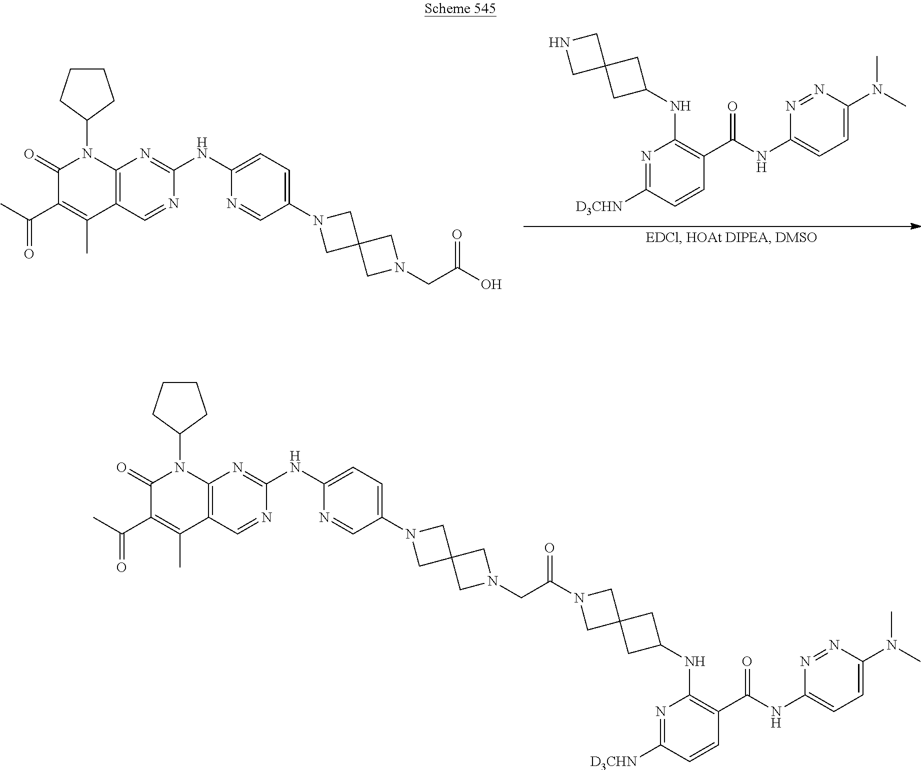

CPD-489 was synthesized following the standard procedure for preparing CPD-143 (6.2 mg, 30% yield) as a yellow solid. MS (ESI) m/z=886.0 [M+H]⁺.

### Example 546. 2-((1-(2-(6-(6-((6-acetyl-8-cyclopentyl-5-methyl-7-oxo-7,8-dihydropyrido[2,3-d]pyrimidin-2-yl)amino)pyridin-3-yl)-2,6-diazaspiro[3.3]heptan-2-yl)acetyl)piperidin-4-yl)amino)-N-(6-(dimethylamino)pyridazin-3-yl)-6-((methyl-d₃)amino)nicotinamide (CPD-490)

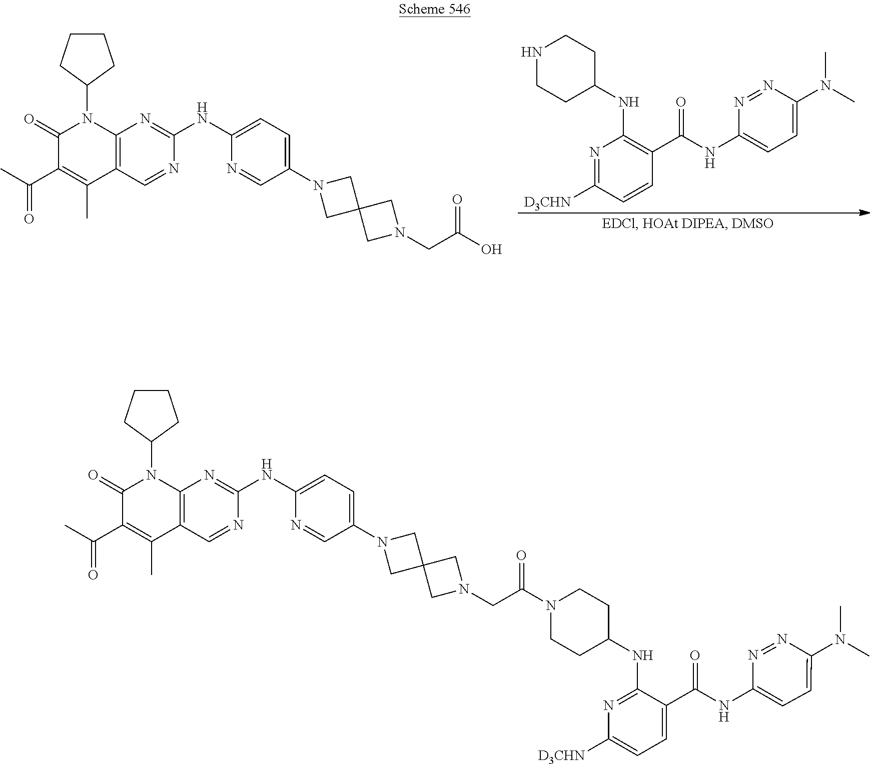

CPD-490 was synthesized following the standard procedure for preparing CPD-143 (9.8 mg, 52% yield) as a yellow solid. MS (ESI) m/z=874.0 [M+H]⁺.

### Example 547. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)piperidin-4-yl)amino)-N-(6-(dimethylamino)pyridazin-3-yl)-6-(methyl-d₃)amino)nicotinamide (CPD-491)

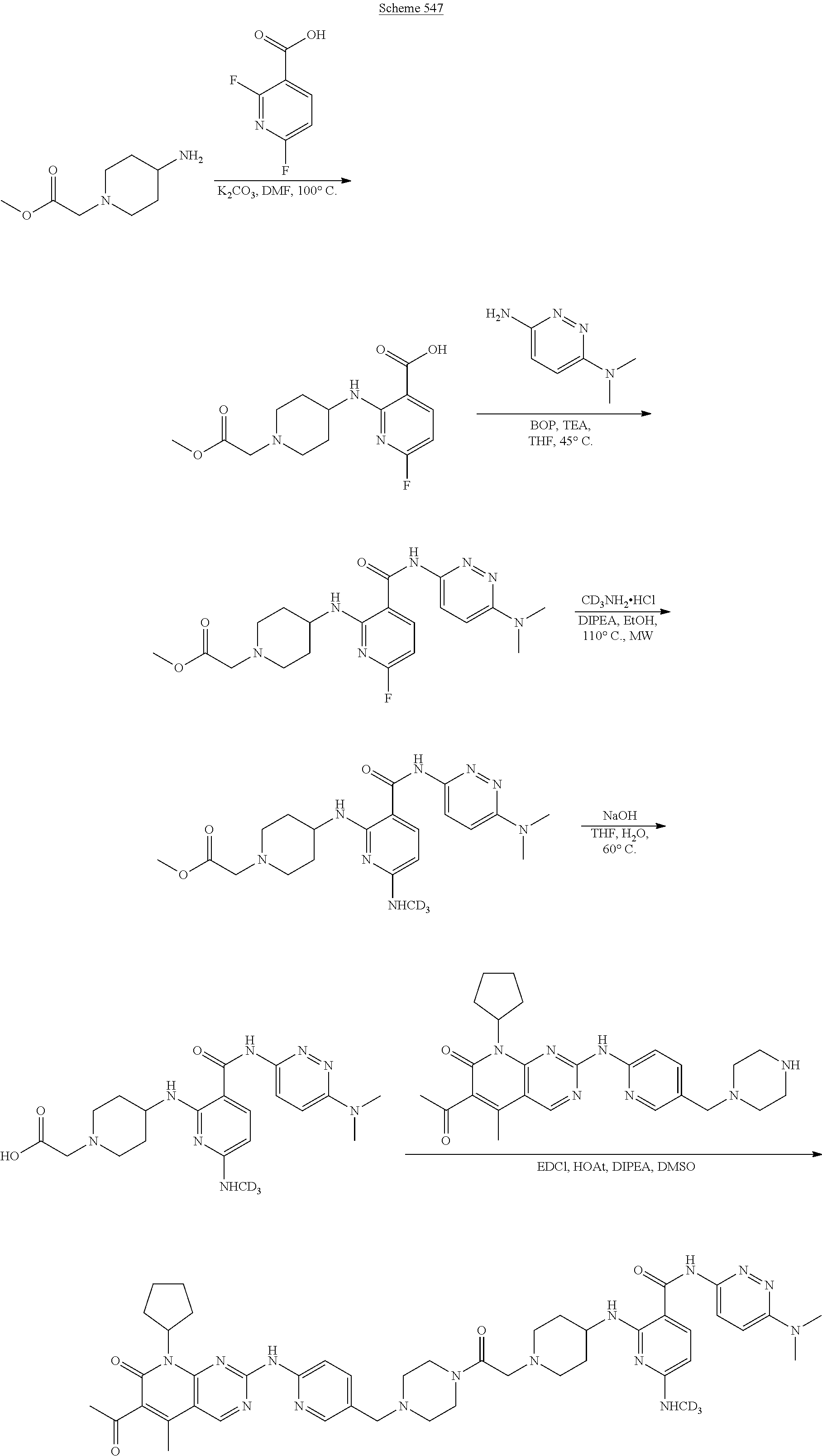

CPD-491 was synthesized following the standard procedures for preparing CPD-82 (3.6 mg, 3% yield over 5 steps) as a yellow solid. MS (ESI) m/z=876.1 [M+H]⁺.

### Example 548. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-ethylnicotinamide (CPD-492)

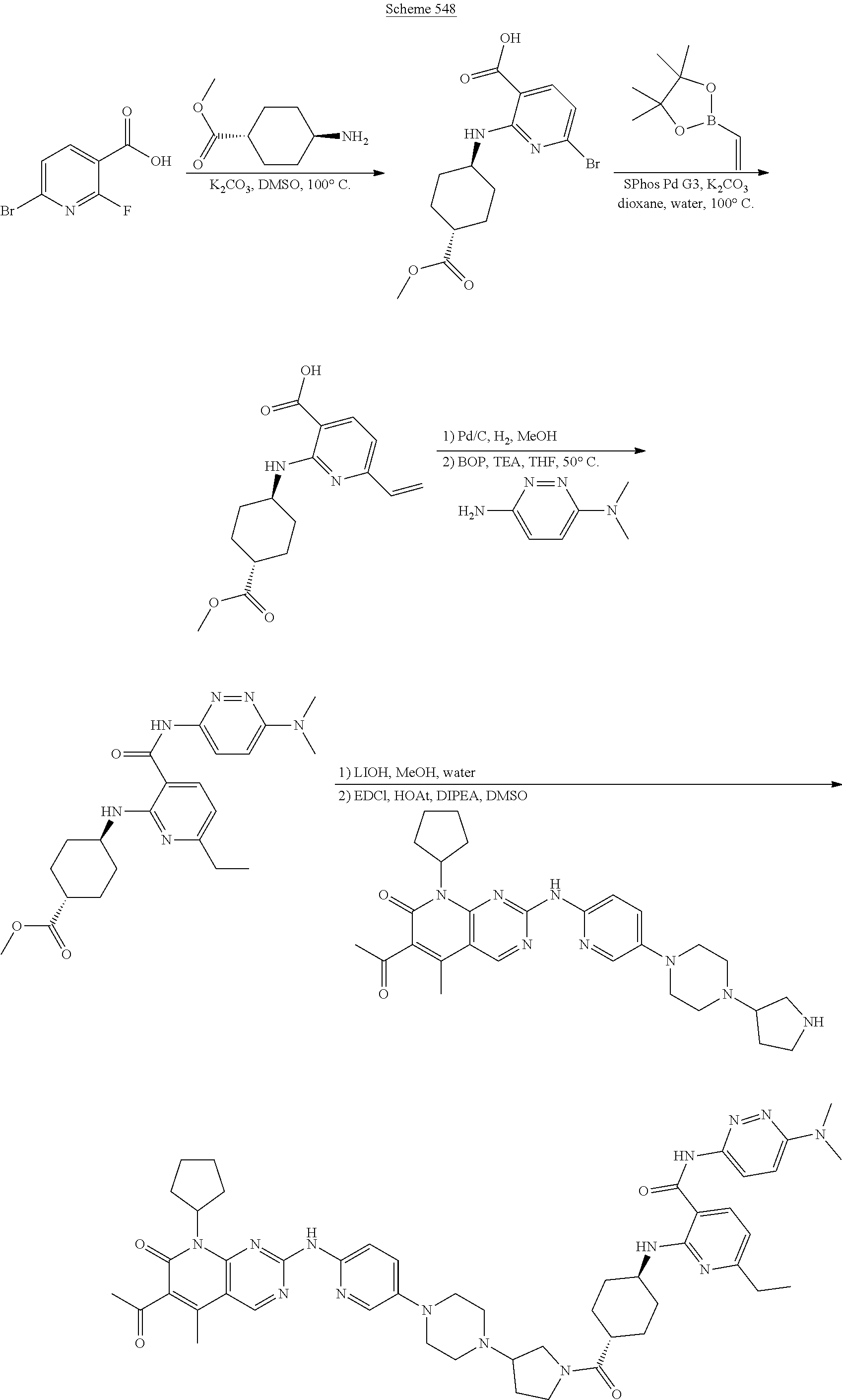

### Step 1. Synthesis of 6-bromo-2-((trans-4-(methoxycarbonyl)cyclohexyl)amino)nicotinic acid

To a solution of 6-bromo-2-fluoronicotinic acid (1 g, 4.55 mmol) in DMSO (20 mL) were added methyl trans-4-aminocyclohexane-1-carboxylate (714.60 mg, 4.55 mmol) and K₂CO₃ (942.33 mg, 6.82 mmol) at rt. The reaction mixture was stirred at 90° C. for 16 h. After cooling down to rt, the mixture was poured into water (200 mL) and extracted with dichloromethane (3×100 mL). The combined organic layers were washed with saturated brine (2×100 mL), dried over anhydrous sodium sulfate, filtered, and evaporated under reduced pressure. The resulting residue was purified by reverse-phase chromatography to give the desired product (800 mg, 49% yield) as a pale white solid. MS (ESI) m/z=357.2 [M+H]⁺.

### Step 2. Synthesis of 2-((trans-4-(methoxycarbonyl)cyclohexyl)amino)-6-vinylnicotinic acid

To a solution of 6-bromo-2-((trans-4-(methoxycarbonyl)cyclohexyl)amino)nicotinic acid (200 mg, 559.91 μmol) in dioxane (4 mL) and water (1 mL) were added 4,4,5,5-tetramethyl-2-vinyl-1,3,2-dioxaborolane (172.47 mg, 1.12 mmol), SPhos Pd G3 (43.69 mg, 55.99 μmol) and K₂CO₃ (77.38 mg, 559.91 μmol) at rt. The mixture was stirred at 100° C. under inert atmosphere for 3 h. After cooling down to rt, the reaction mixture was poured into water (50 mL) and extracted with dichloromethane (3×25 mL). The combined organic layers were washed with saturated brine (30 mL), dried over anhydrous sodium sulfate, filtered, and evaporated under reduced pressure. The resulting residue was purified by silica gel chromatography to give the desired product (145 mg, 85% yield) as a pale-white solid. MS (ESI) m/z=305.3 [M+H]⁺.

### Step 3. Synthesis of 6-ethyl-2-((trans-4-(methoxycarbonyl)cyclohexyl)amino)nicotinic acid

To a solution of 2-((trans-4-(methoxycarbonyl)cyclohexyl)amino)-6-vinylnicotinic acid (130 mg, 427.15 μmol) in MeOH (5 mL) was added 5% Pd/C (13 mg). The reaction mixture was stirred at rt under hydrogen atmosphere for 2 h. The reaction mixture was filtered and concentrated under reduced pressure. The residue was purified by silica gel chromatography to give the desired product (90 mg, 69% yield) as a white solid. MS (ESI) m/z=307.2 [M+H]⁺.

### Step 4. Synthesis of methyl trans-4-((3-((6-(dimethylamino)pyridazin-3-yl)carbamoyl)-6-ethylpyridin-2-yl)amino)cyclohexane-1-carboxylate

To a solution of 6-ethyl-2-((trans-4-(methoxycarbonyl)cyclohexyl)amino)nicotinic acid (20 mg, 65.28 μmol) in THF (2 mL) were added N³,N³-dimethylpyridazine-3,6-diamine (10.82 mg, 78.34 μmol), BOP (43.31 mg, 97.93 μmol) and TEA (33.03 mg, 326.42 mol). The mixture was stirred at 50 TC for 16 h. The reaction mixture was poured into water (20 mL) and extracted with dichloromethane (3×20 mL). The combined organic layers were washed with saturated brine (15 mL), dried over anhydrous sodium sulfate, filtered, and evaporated under reduced pressure. The resulting residue was purified by silica gel chromatography to give the desired product (10 mg, 36% yield) as a pale-white solid. MS (ESI) m/z=427.3 [M+H]⁺.

### Step 5. Synthesis of trans-4-((3-((6-(dimethylamino)pyridazin-3-yl)carbamoyl)-6-ethylpyridin-2-yl)amino)cyclohexane-1-carboxylic acid

To a solution of methyl trans-4-((3-((6-(dimethylamino)pyridazin-3-yl)carbamoyl)-6-ethylpyridin-2-yl)amino)cyclohexane-1-carboxylate (10 mg, 23.45 μmol) in water (1 mL) and MeOH (1 mL) were added LiOH (5.62 mg, 234.46 μmol) at rt. The mixture was stirred at rt for 16 h. The reaction mixture was purified by reverse-phase chromatography to give the desired product (7 mg, 72% yield) as a pale-white solid. MS (ESI) m/z=413.3 [M+H]⁺.

### Step 6. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-ethylnicotinamide

To a mixture of tran-4-((3-((6-(dimethylamino)pyridazin-3-yl)carbamoyl)-6-ethylpyridin-2-yl)amino)cyclohexane-1-carboxylic acid (7 mg, 16.97 μmol), EDCI (4.88 mg, 25.46 mol), HOAt (3.46 mg, 25.46 μmol) and 6-acetyl-8-cyclopentyl-5-methyl-2-((5-(4-(pyrolidin-3-yl)piperazin-1-yl)pyridin-2-yl)amino)pyrido[2,3-d]pyrimidin-7(8H)-one (10.52 mg, 20.36 μmol) in DMSO (1 mL) was added DIPEA (10.97 mg, 84.85 μmol). The mixture was stirred at rt for 16 h, before it was purified by prep-HPLC to provide the desired product (6.5 mg, 37% yield) as a yellow solid. MS (ESI) m/z=911.9 [M+H]⁺.

### Example 549. 2-((3-((2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)methyl)cyclobutyl)amino)-N-(6-(dimethylamino)pyridazin-3-yl)-6-(isopropylamino)nicotinamide (CPD-493)

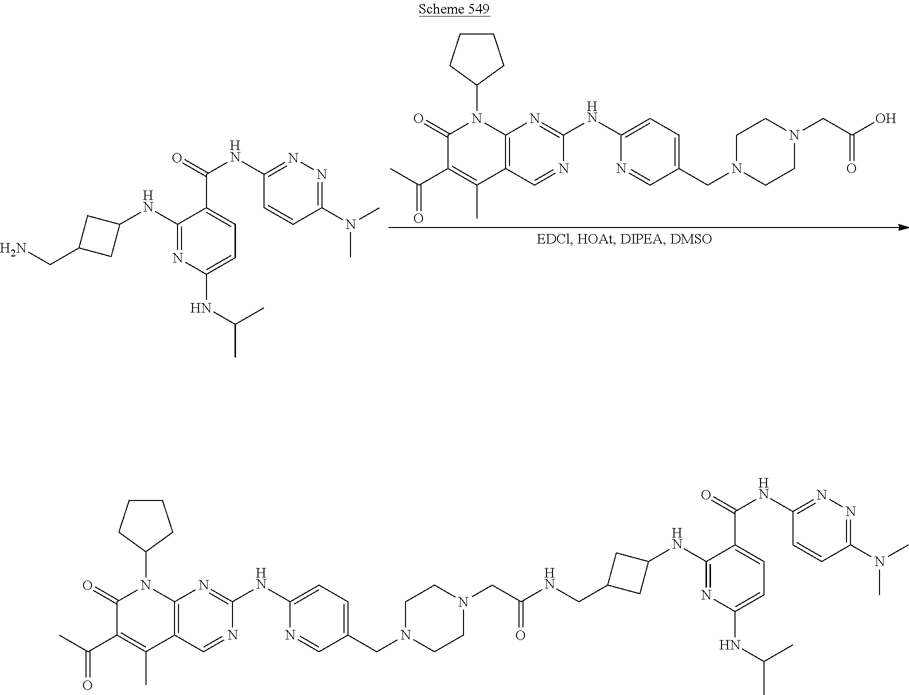

CPD-493 was synthesized following the standard procedure for preparing CPD-143 (6.7 mg, 20% yield) as a yellow solid. MS (ESI) m/z=900.9 [M+H]⁺.

### Example 550. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carboxamido)cyclobutyl)amino)-N-(6-(dimethylamino)pyridazin-3-yl)-6-((methyl-d₃)amino)nicotinamide (CPD-494)

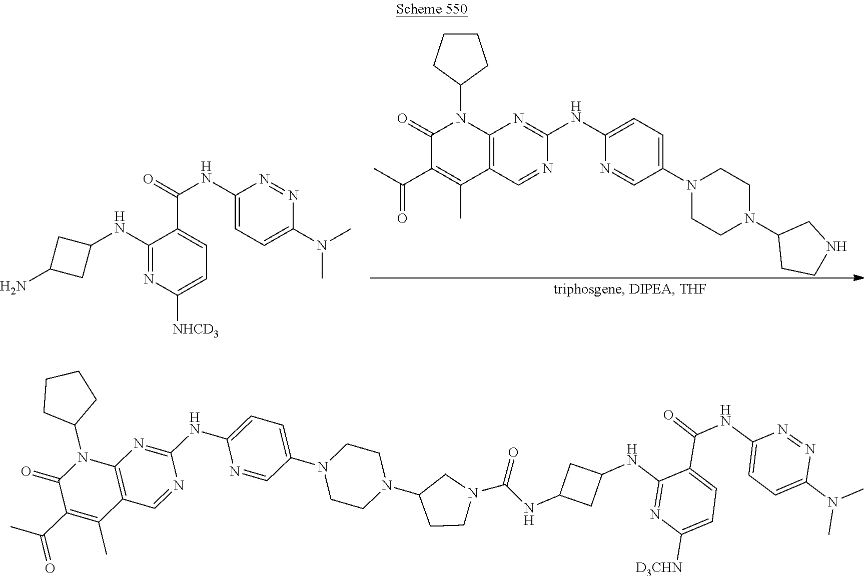

CPD-494 was synthesized following the standard procedure for preparing CPD-469 (0.8 mg, 5% yield) as a yellow solid. MS (ESI) m/z=903.1 [M+H]⁺.

### Example 551. 2-((4-(4-((6-((6-acetyl-8-cyclopentyl-5-methyl-7-oxo-7,8-dihydropyrido[2,3-d]pyrimidin-2-yl)amino)pyridin-3-yl)methyl)piperazine-1-carbonyl)cyclohexyl)amino)-N-(6-(dimethylamino)pyridazin-3-yl)-6-((methyl-d₃)amino)nicotinamide (CPD-495)

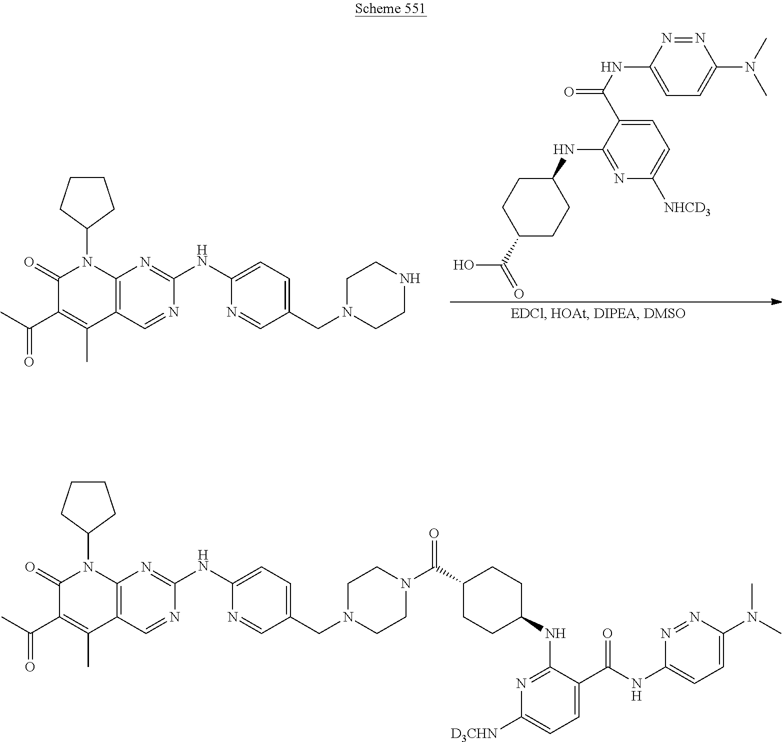

CPD-495 was synthesized following the standard procedure for preparing CPD-143 (4.1 mg, 33% yield) as a yellow solid. MS (ESI) m/z=860.9 [M+H]⁺.

### Example 552. 2-((3-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetamido)bicyclo[1.1.1]pentan-1-yl)amino)-N-(6-(dimethylamino)pyridazin-3-yl)-6-(isopropylamino)nicotinamide (CPD-496)

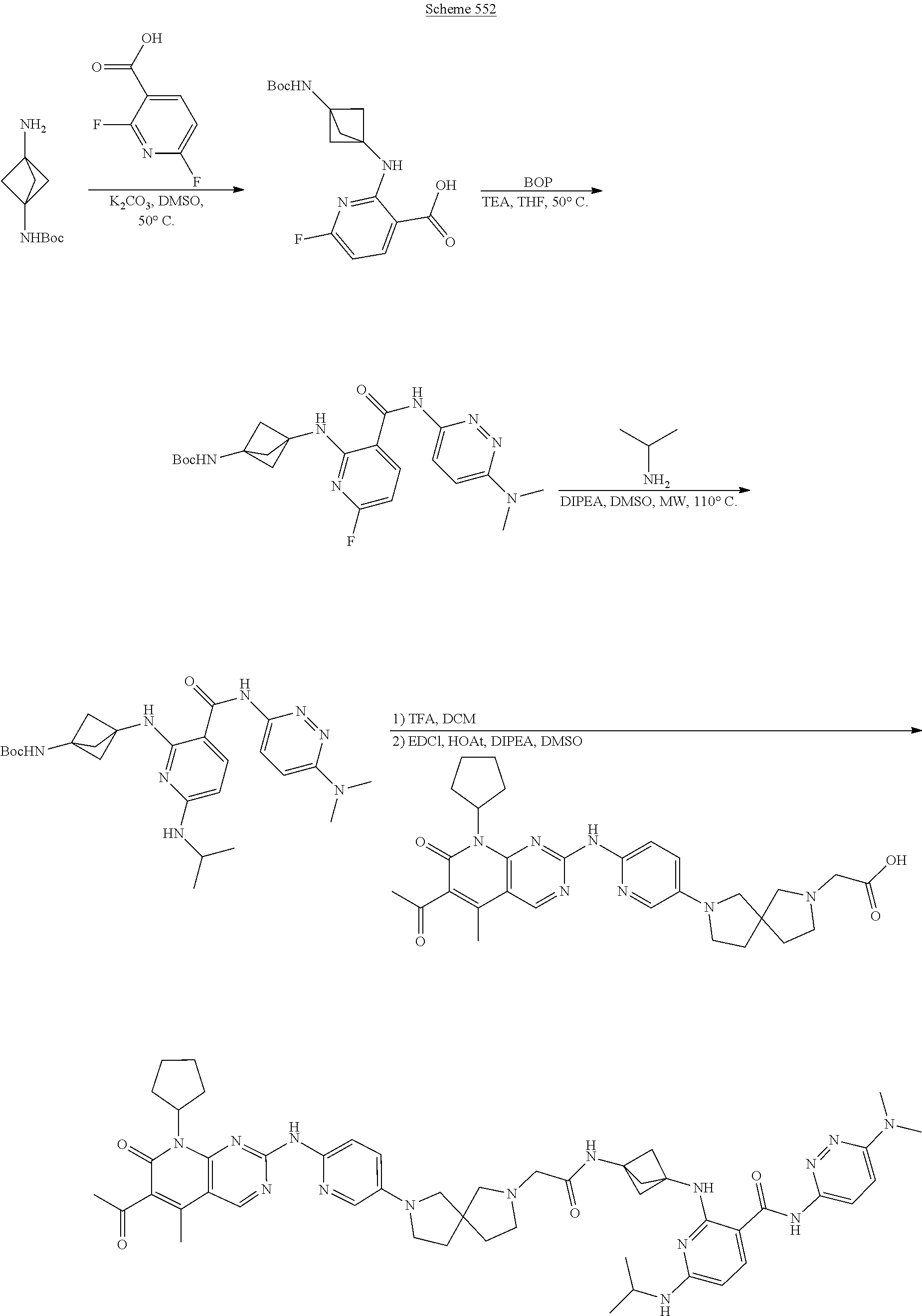

CPD-496 was synthesized following the standard procedures for preparing CPD-145 (7.7 mg, 5% yield over 5 steps) as a yellow solid. MS (ESI) m/z=924.9 [M+H]⁺.

### Example 553. 2-((2-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)-2-oxoethyl)-2-azaspiro[3.3]heptan-6-yl)amino)-N-(6-(dimethylamino)pyridazin-3-yl)-6-(isopropylamino) nicotinamide (CPD-497)

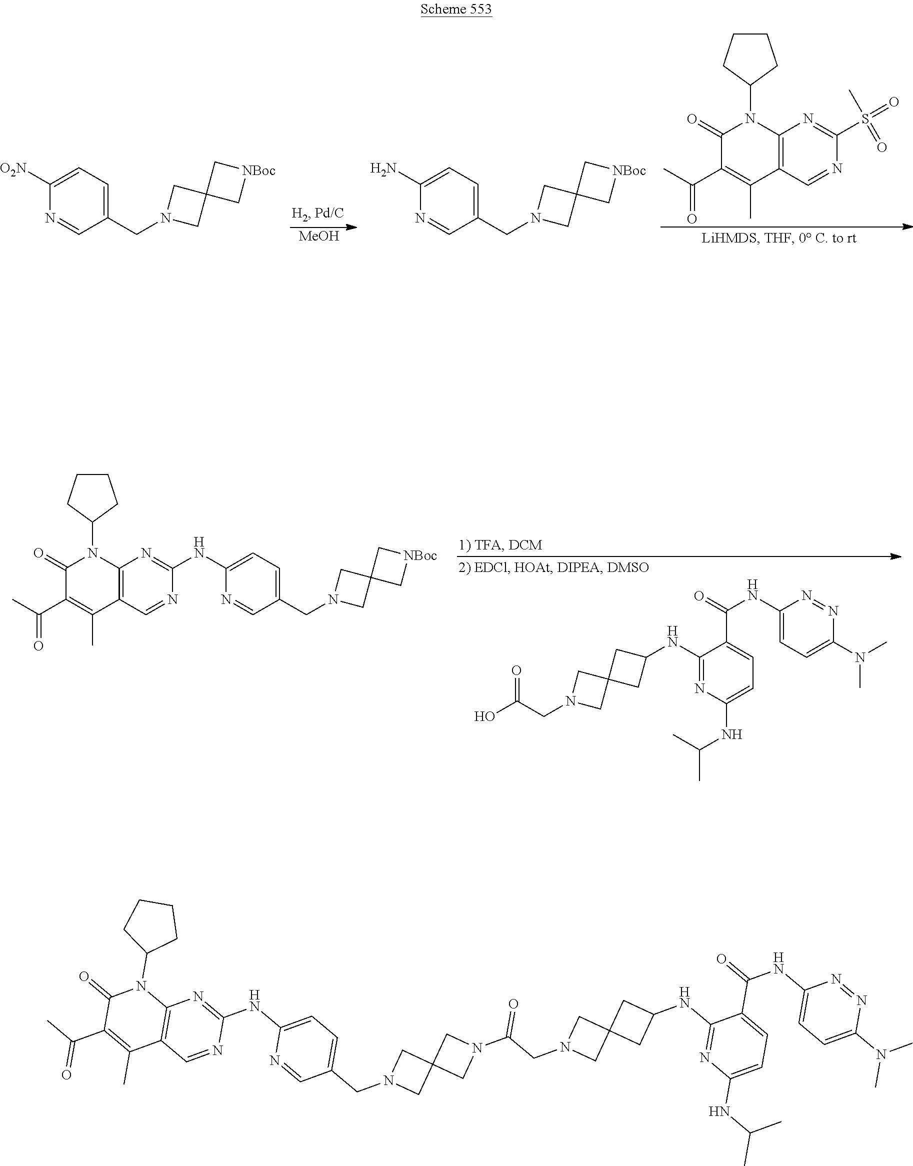

CPD-497 was synthesized following the standard procedures for preparing CPD-418 (1.3 mg, 2% yield over 4 steps) as a yellow solid. MS (ESI) m/z=924.9 [M+H]⁺.

### Example 554. 2-((1-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)-2-oxoethyl)piperidin-4-yl)amino)-N-(6-(dimethylamino)pyridazin-3-yl)-6-(isopropylamino)nicotinamide (CPD-498)

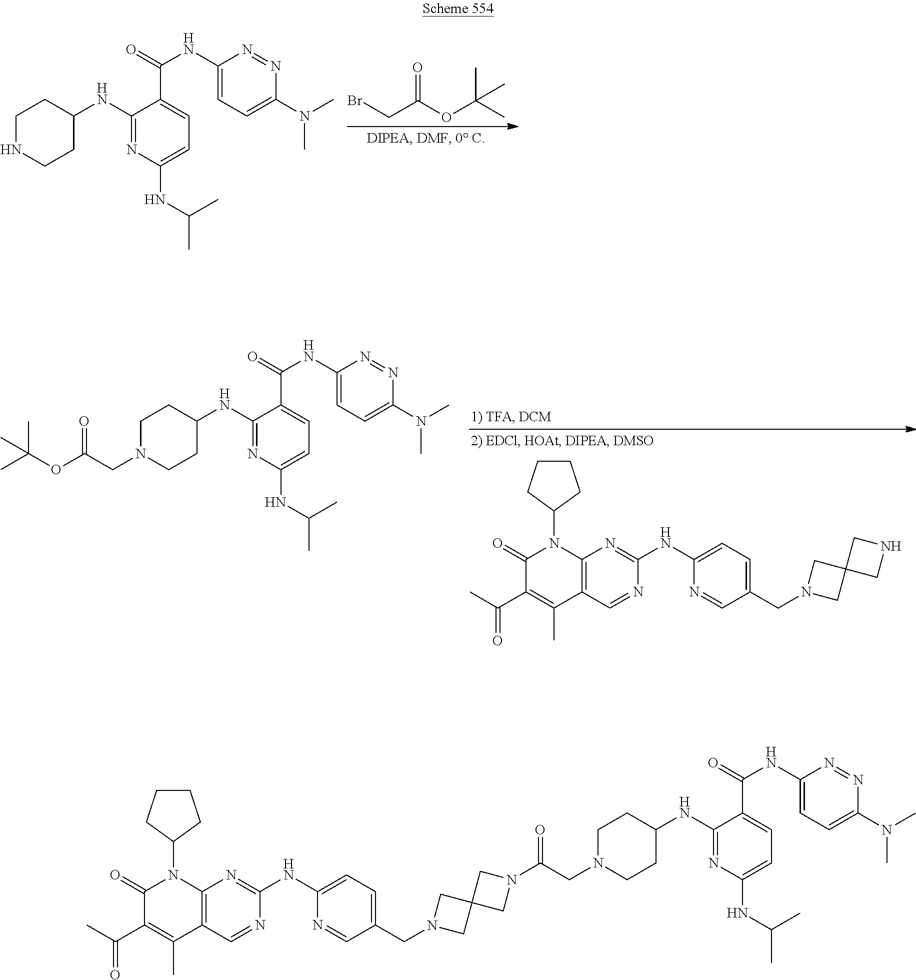

CPD-498 was synthesized following the standard procedures for preparing CPD-418 (16.1 mg, 23% yield over 3 steps) as a yellow solid. MS (ESI) m/z=912.8 [M+H]⁺.

### Example 555. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-isopropylnicotinamide (CPD-499)

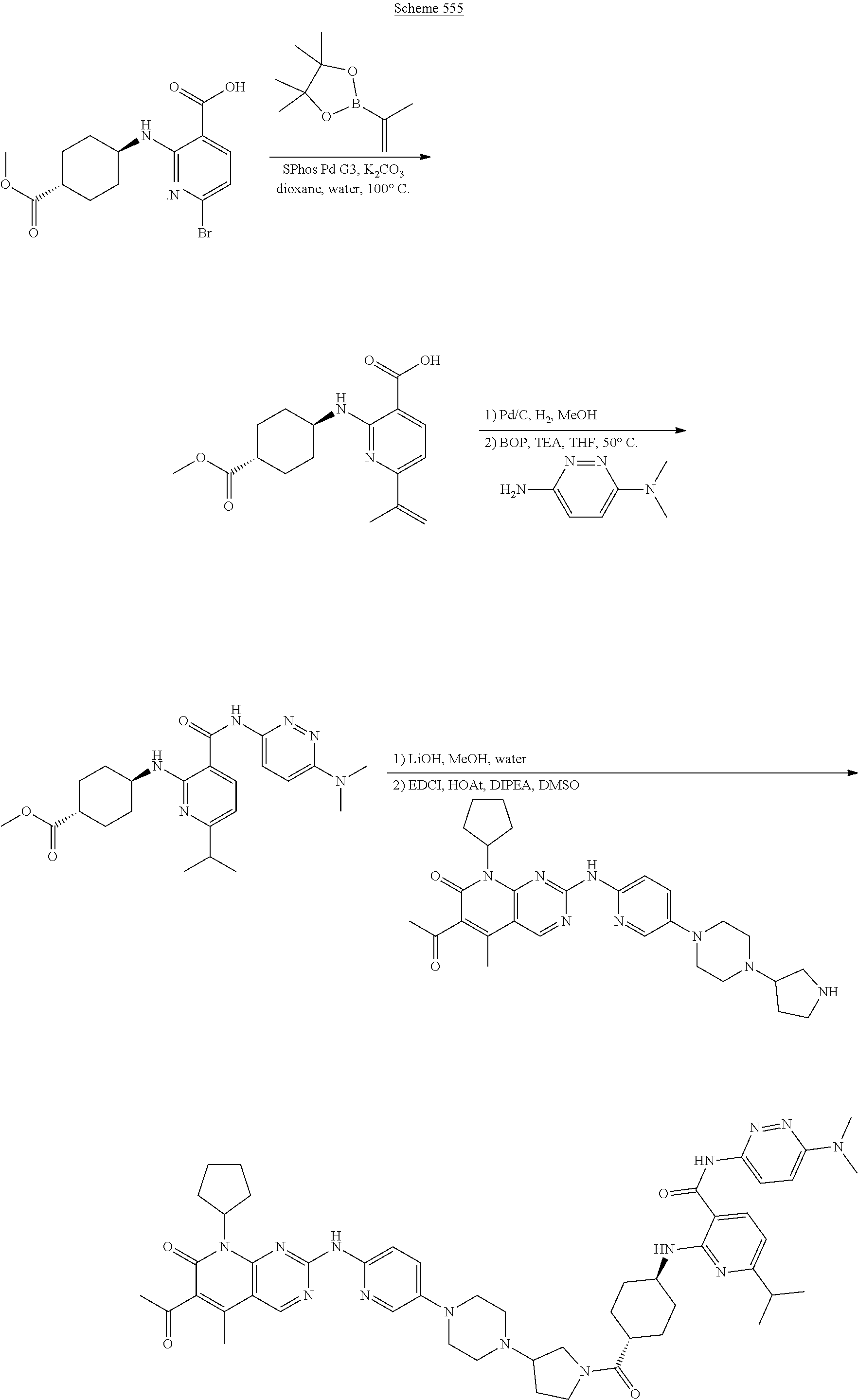

CPD-499 was synthesized following the standard procedures for preparing CPD-492 (4.6 mg, 3% yield over 5 steps) as a yellow solid. MS (ESI) m/z=926.1 [M+H]⁺.

### Example 556. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-(isopropylamino)-N-(6-methoxypyridazin-3-yl)nicotinamide (CPD-500)

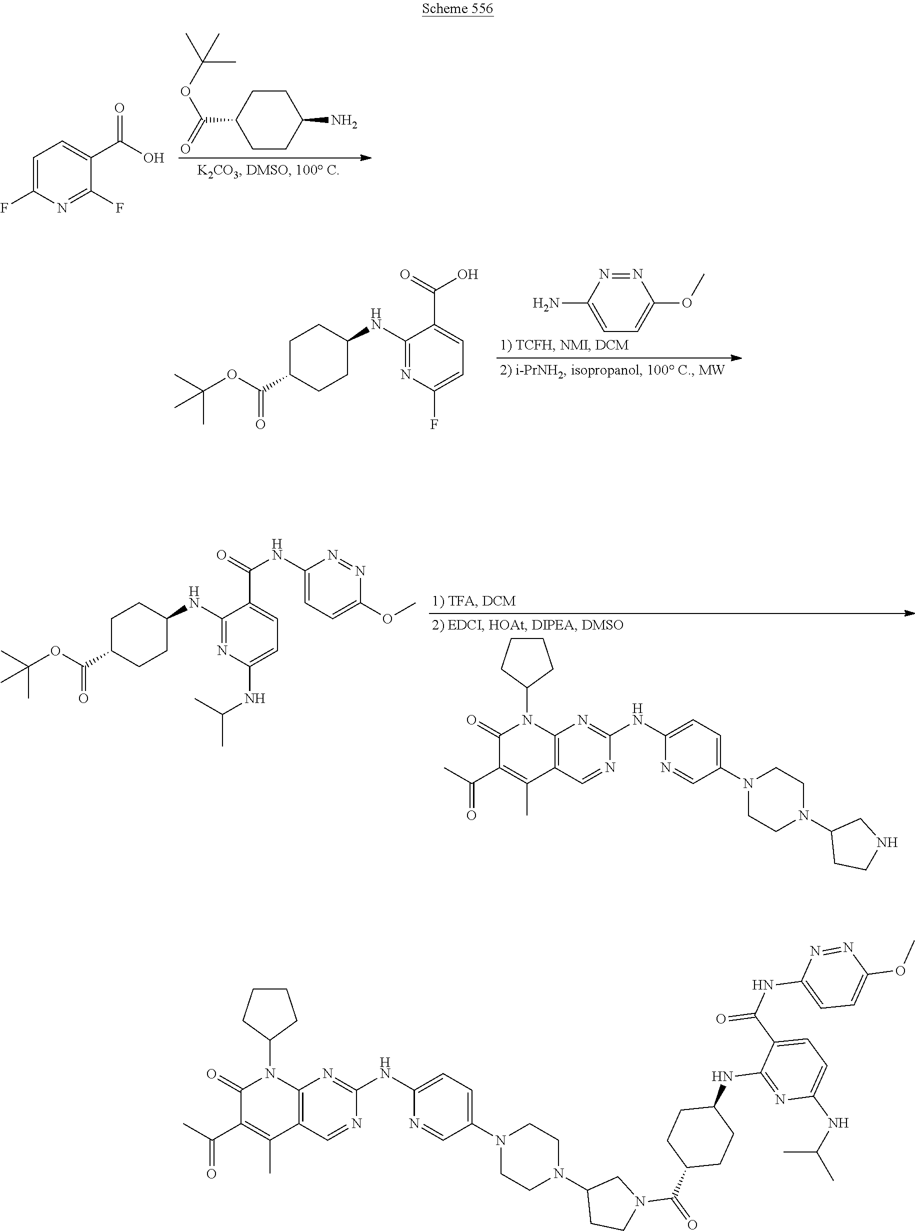

CPD-500 was synthesized following the standard procedures for preparing CPD-145 (1.1 mg, 1% yield over 5 steps) as a yellow solid. MS (ESI) m/z=927.9 [M+H]⁺.

### Example 557. 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)cyclopentyl)amino)-N-(6-(dimethylamino)pyridazin-3-yl)-6-(isopropylamino)nicotinamide (CPD-501)

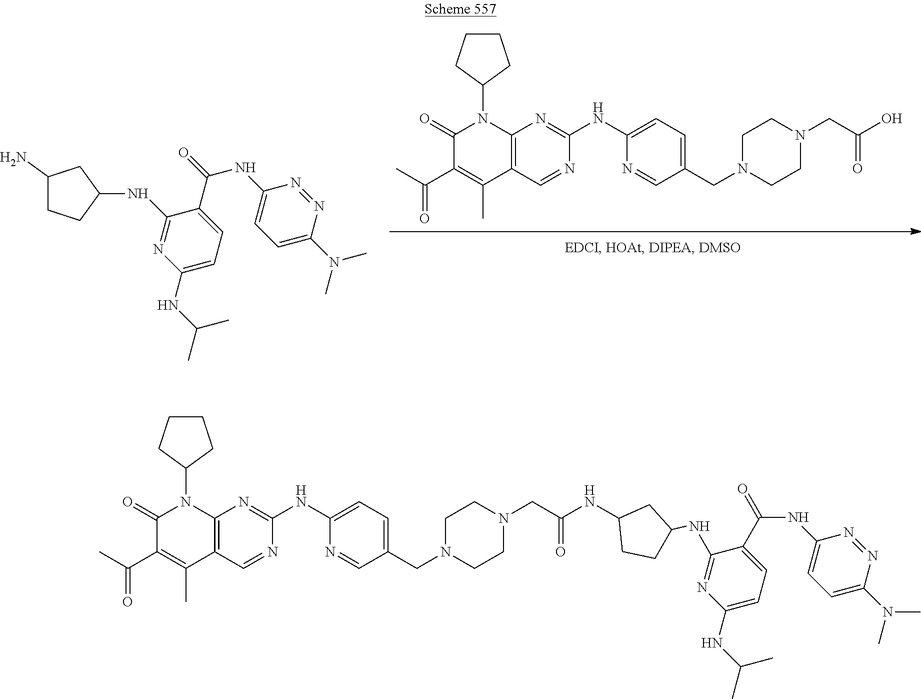 [SMILES: CC(C)Nc1ccc(C(=O)Nc2ccc(N(C)C)nn2)c(NC2CCC(N)C2)n1 ; CC(=O)c1c(C)c2cnc(Nc3ccc(CN4CCN(CC(=O)O)CC4)cn3)nc2n(C2CCCC2)c1=O ; CC(=O)c1c(C)c2cnc(Nc3ccc(CN4CCN(CC(=O)NC5CCC(Nc6nc(NC(C)C)ccc6C(=O)Nc6ccc(N(C)C)nn6)C5)CC4)cn3)nc2n(C2CCCC2)c1=O]

CPD-501 was synthesized following the standard procedure for preparing CPD-143 (3.0 mg, 12% yield) as a yellow solid. MS (ESI) m/z=901.0 [M+H]⁺.

### Example 558. 2-((3-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)bicyclo[1.1.1]pentan-1-yl)amino)-N-(6-(dimethylamino)pyridazin-3-yl)-6-(isopropylamino)nicotinamide (CPD-502)

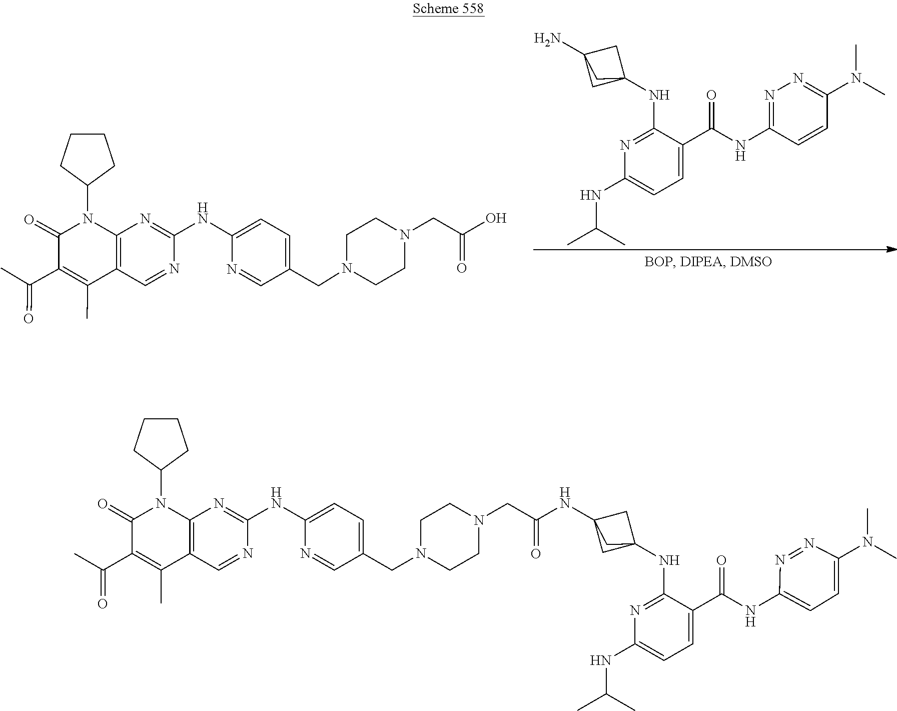 [SMILES: CC(=O)c1c(C)c2cnc(Nc3ccc(CN4CCN(CC(=O)O)CC4)cn3)nc2n(C2CCCC2)c1=O ; CC(C)Nc1ccc(C(=O)Nc2ccc(N(C)C)nn2)c(NC23CC(N)(C2)C3)n1 ; CC(=O)c1c(C)c2cnc(Nc3ccc(CN4CCN(CC(=O)NC56CC(Nc7nc(NC(C)C)ccc7C(=O)Nc7ccc(N(C)C)nn7)(C5)C6)CC4)cn3)nc2n(C2CCCC2)c1=O]

CPD-502 was synthesized following the standard procedure for preparing CPD-261 (6.0 mg, 23% yield) as a yellow solid. MS (ESI) m/z=898.9 [M+H]⁺.

### Example 559. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)propanamido)bicyclo[1.1.1]pentan-1-yl)amino)-N-(6-(dimethylamino)pyridazin-3-yl)-6-(isopropylamino)nicotinamide (CPD-503)

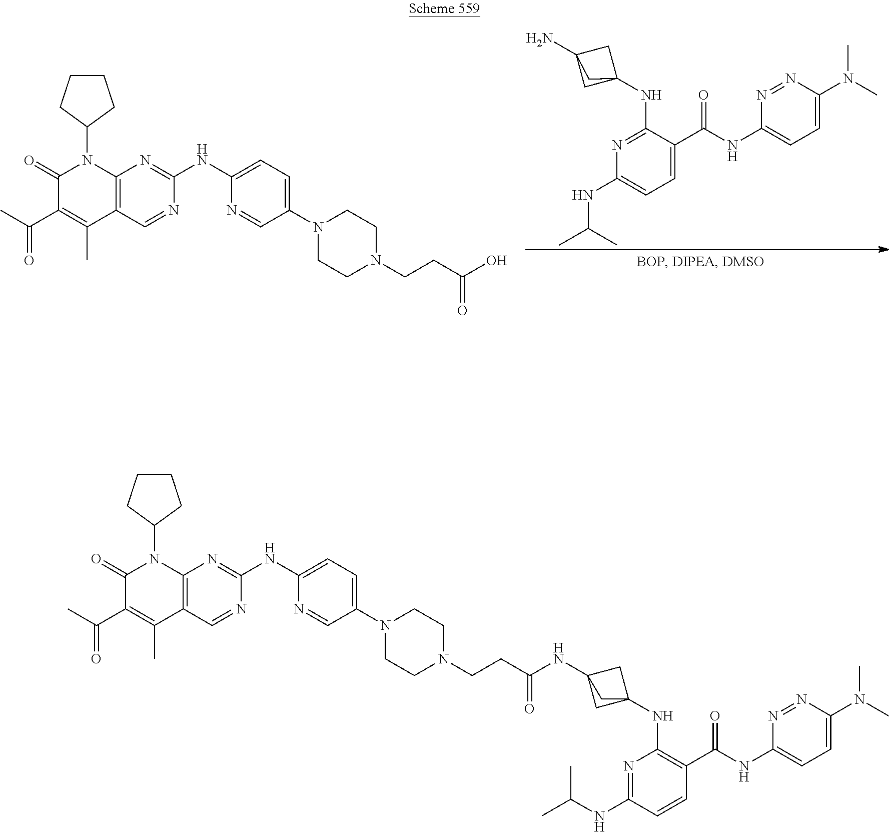 [SMILES: CC(=O)c1c(C)c2cnc(Nc3ccc(N4CCN(CCC(=O)O)CC4)cn3)nc2n(C2CCCC2)c1=O ; CC(C)Nc1ccc(C(=O)Nc2ccc(N(C)C)nn2)c(NC23CC(N)(C2)C3)n1 ; CC(=O)c1c(C)c2cnc(Nc3ccc(N4CCN(CCC(=O)NC56CC(Nc7nc(NC(C)C)ccc7C(=O)Nc7ccc(N(C)C)nn7)(C5)C6)CC4)cn3)nc2n(C2CCCC2)c1=O]

CPD-503 was synthesized following the standard procedure for preparing CPD-261 (7.0 mg, 27% yield) as a yellow solid. MS (ESI) m/z=898.9 [M+H]⁺.

### Example 560. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)butanamido)bicyclo[1.1.1]pentan-1-yl)amino)-N-(6-(dimethylamino)pyridazin-3-yl)-6-(isopropylamino)nicotinamide (CPD-504)

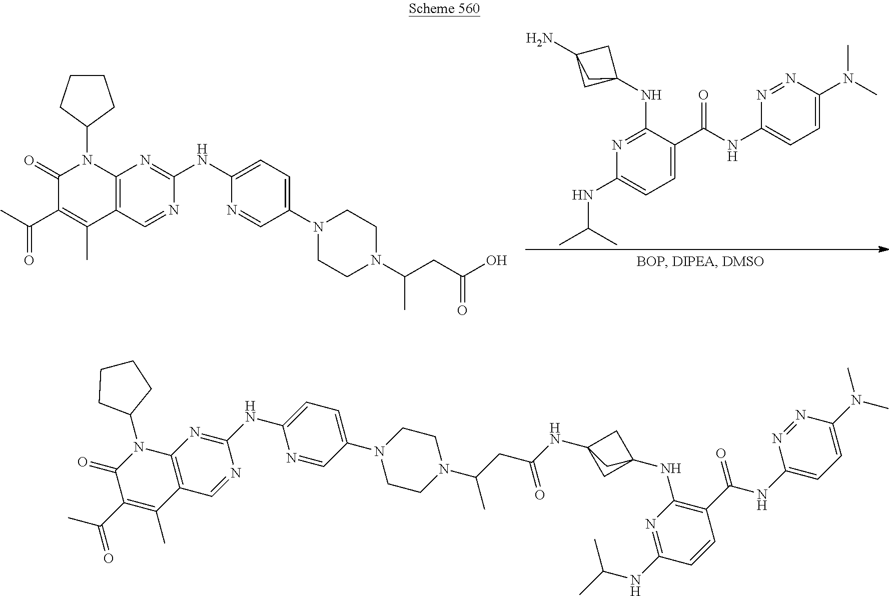 [SMILES: CC(=O)c1c(C)c2cnc(Nc3ccc(N4CCN(C(C)CC(=O)O)CC4)cn3)nc2n(C2CCCC2)c1=O ; CC(C)Nc1ccc(C(=O)Nc2ccc(N(C)C)nn2)c(NC23CC(N)(C2)C3)n1 ; CC(=O)c1c(C)c2cnc(Nc3ccc(N4CCN(C(C)CC(=O)NC56CC(Nc7nc(NC(C)C)ccc7C(=O)Nc7ccc(N(C)C)nn7)(C5)C6)CC4)cn3)nc2n(C2CCCC2)c1=O]

CPD-504 was synthesized following the standard procedure for preparing CPD-261 (12.0 mg, 46% yield) as a yellow solid. MS (ESI) m/z=912.9 [M+H]⁺.

### Example 561. 2-((3-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)acetamido)bicyclo[1.1.1]pentan-1-yl)amino)-N-(6-(dimethylamino)pyridazin-3-yl)-6-(isopropylamino)nicotinamide (CPD-505)

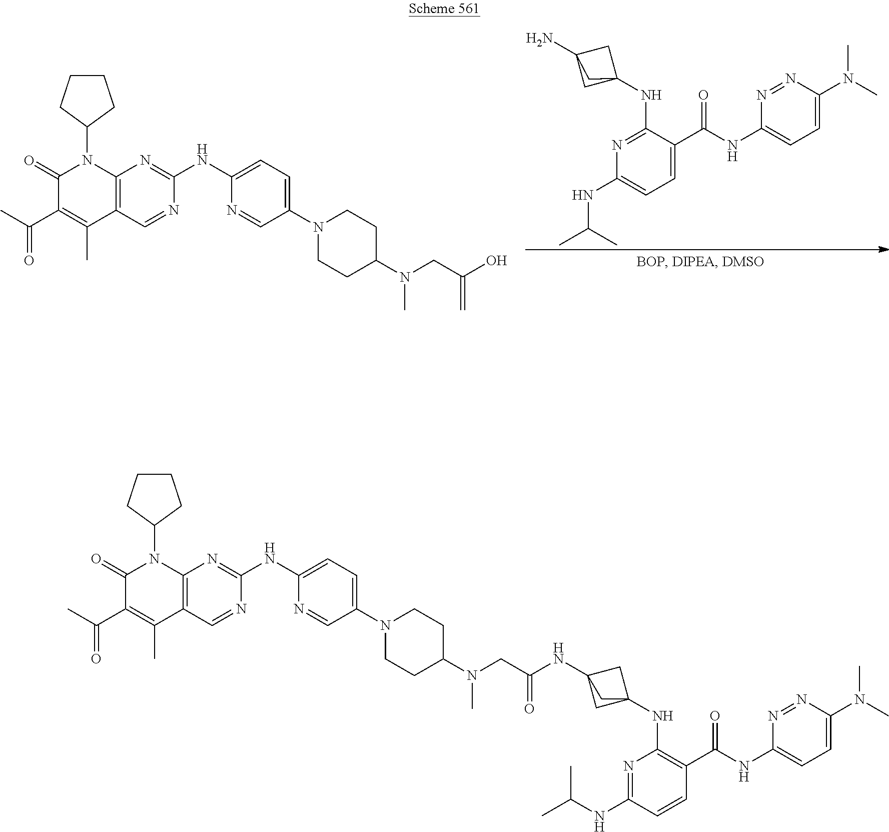 [SMILES: C=C(O)CN(C)C1CCN(c2ccc(Nc3ncc4c(C)c(C(C)=O)c(=O)n(C5CCCC5)c4n3)nc2)CC1 ; CC(C)Nc1ccc(C(=O)Nc2ccc(N(C)C)nn2)c(NC23CC(N)(C2)C3)n1 ; CC(=O)c1c(C)c2cnc(Nc3ccc(N4CCC(N(C)CC(=O)NC56CC(Nc7nc(NC(C)C)ccc7C(=O)Nc7ccc(N(C)C)nn7)(C5)C6)CC4)cn3)nc2n(C2CCCC2)c1=O]

CPD-505 was synthesized following the standard procedure for preparing CPD-261 (10.0 mg, 39% yield) as a yellow solid. MS (ESI) m/z=912.9 [M+H]⁺.

### Example 562. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)carbamoyl)bicyclo[1.1.1]pentan-1-yl)amino)-N-(6-(dimethylamino)pyridazin-3-yl)-6-(isopropylamino)nicotinamide (CPD-506)

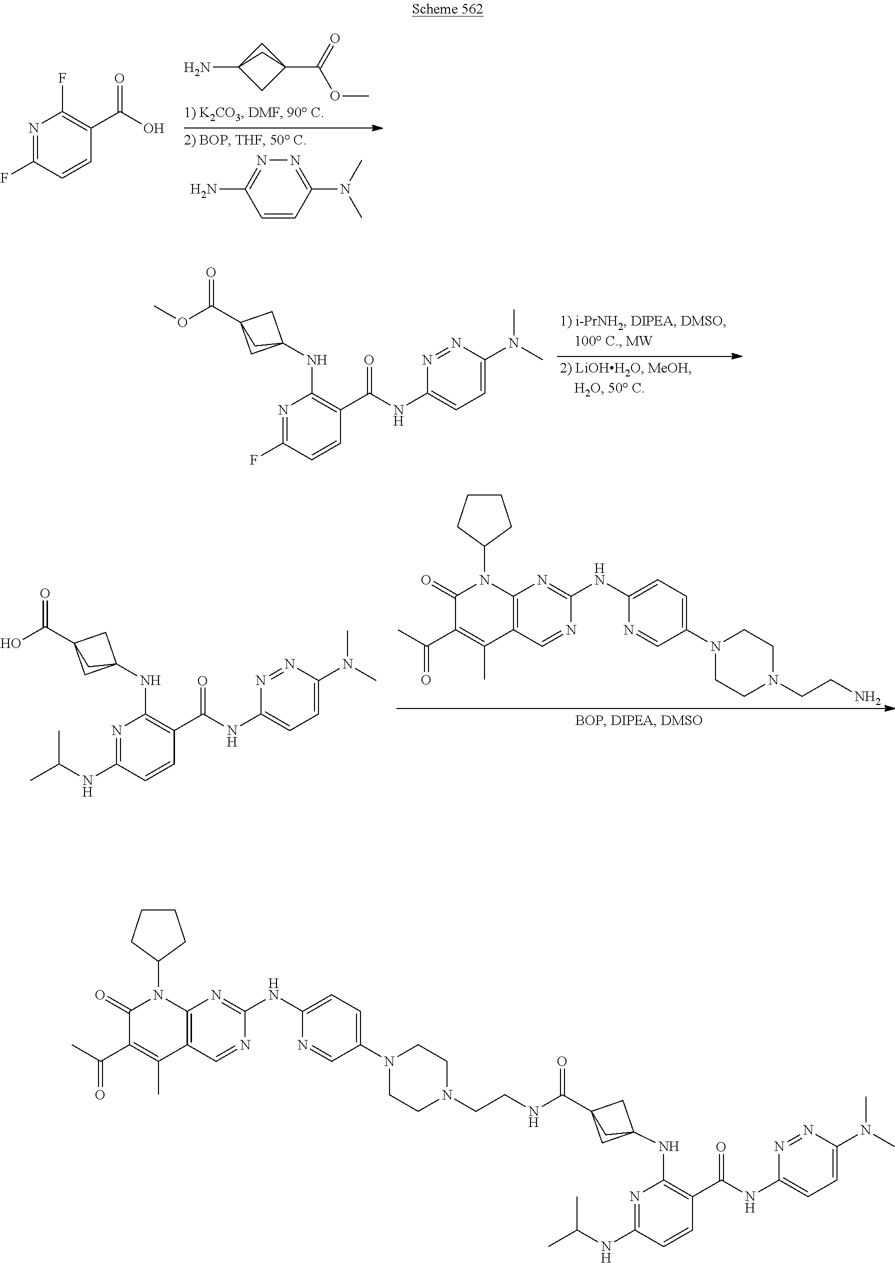

CPD-506 was synthesized following the standard procedures for preparing CPD-82 (5.0 mg, 1% yield over 5 steps) as a yellow solid. MS (ESI) m/z=898.9 [M+H]⁺.

### Example 563. 2-((2-(2-(7-((6-((6-acetyl-8-cyclopentyl-5-methyl-7-oxo-7,8-dihydropyrido[2,3-d]pyrimidin-2-yl)amino)pyridin-3-yl)methyl)-2,7-diazaspiro[4.4]nonan-2-yl)-2-oxoethyl)-2-azaspiro[3.3]heptan-6-yl)amino)-N-(6-(dimethylamino)pyridazin-3-yl)-6-((methyl-d₃)amino)nicotinamide (CPD-507)

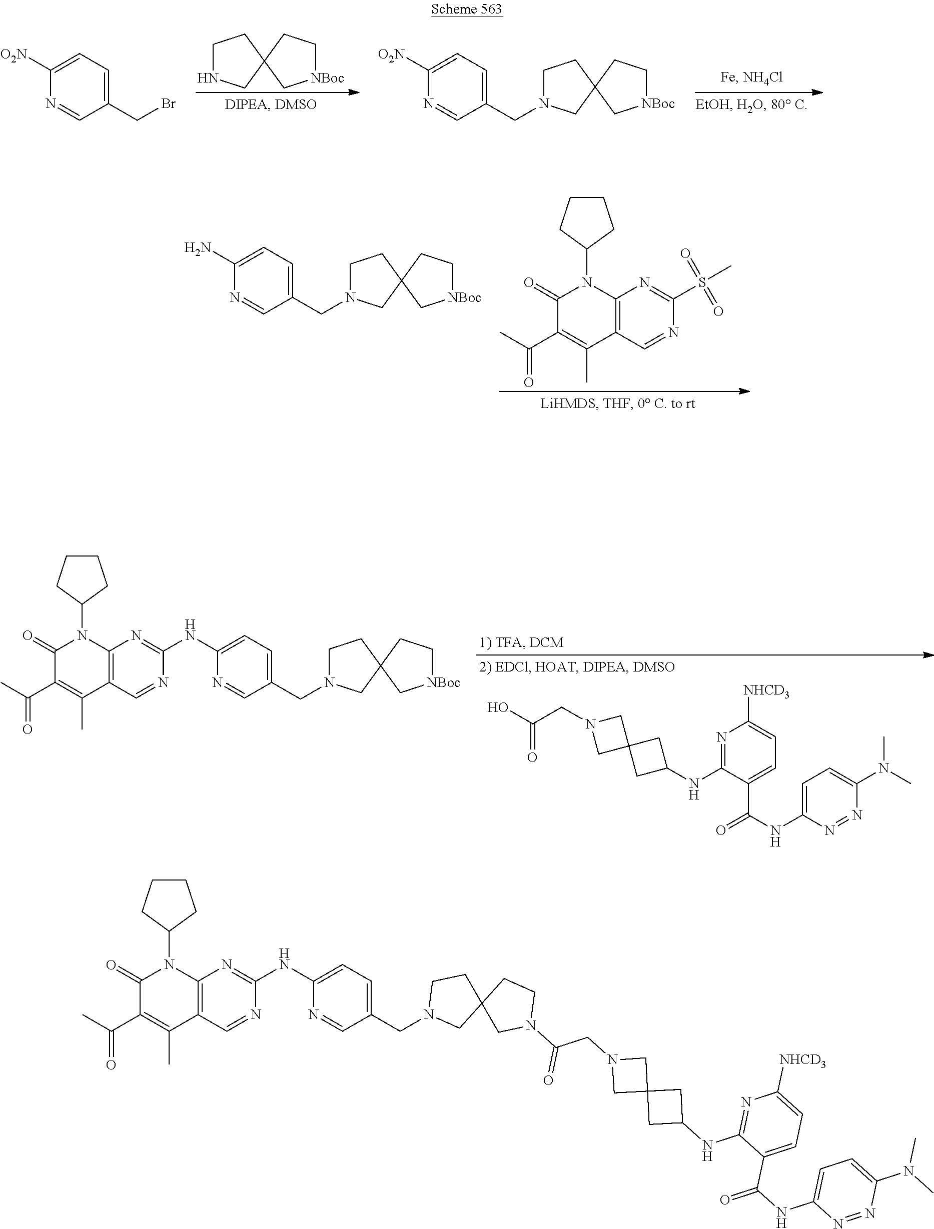

CPD-507 was synthesized following the standard procedures for preparing CPD-418 (2. 1 mg, 1% yield over 5 steps) as a yellow solid. MS (ESI) m/z=927.8 [M+H]⁺.

### Example 564. 2-((2-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)acetyl)-2-azaspiro[3.3]heptan-6-yl)amino)-N-(6-(dimethylamino)pyridazin-3-yl)-6-(isopropylamino)nicotinamide (CPD-508)

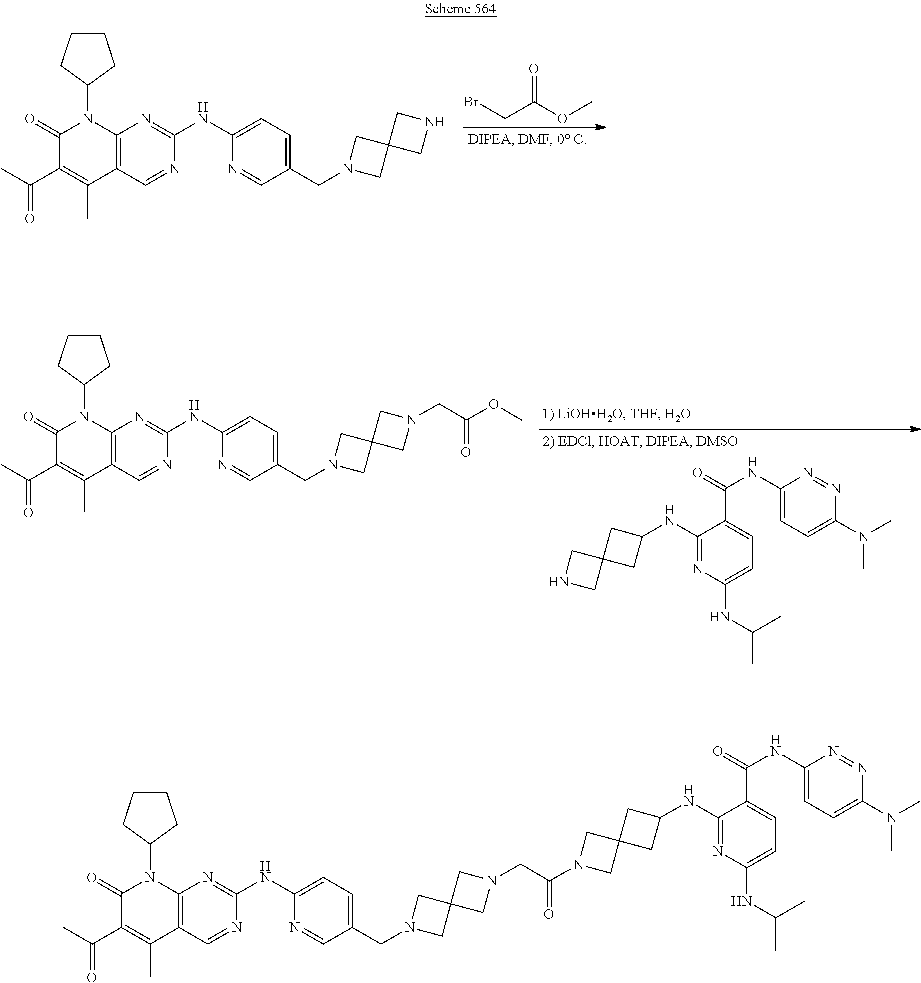

CPD-508 was synthesized following the standard procedures for preparing CPD-392 (8.5 mg, 12% yield over 3 steps) as a yellow solid. MS (ESI) m/z=924.8 [M+H]⁺.

### Example 565. 2-((1-(2-(6-((6-((6-acetyl-8-cyclopentyl-5-methyl-7-oxo-7,8-dihydropyrido[2,3-d]pyrimidin-2-yl)amino)pyridin-3-yl)methyl)-2,6-diazaspiro[3.3]heptan-2-yl)acetyl)piperidin-4-yl)amino)-N-(6-(dimethylamino)pyridazin-3-yl)-6-(isopropylamino)nicotinamide (CPD-509)

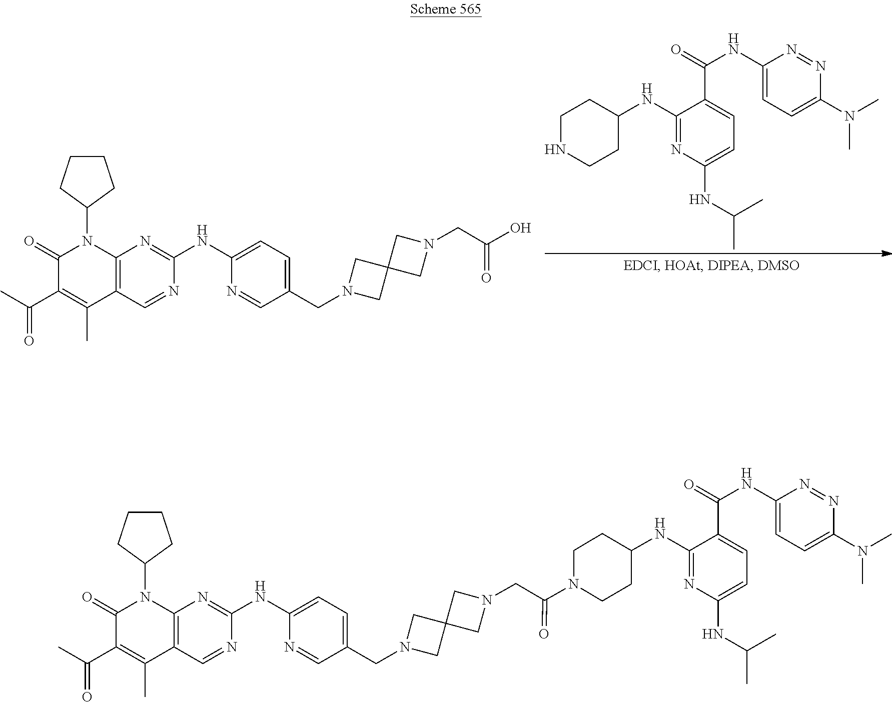 [SMILES: CC(=O)c1c(C)c2cnc(Nc3ccc(CN4CC5(CN(CC(=O)O)C5)C4)cn3)nc2n(C2CCCC2)c1=O ; CC(C)Nc1ccc(C(=O)Nc2ccc(N(C)C)nn2)c(NC2CCNCC2)n1 ; CC(=O)c1c(C)c2cnc(Nc3ccc(CN4CC5(CN(CC(=O)N6CCC(Nc7nc(NC(C)C)ccc7C(=O)Nc7ccc(N(C)C)nn7)CC6)C5)C4)cn3)nc2n(C2CCCC2)c1=O]

CPD-509 was synthesized following the standard procedure for preparing CPD-143 (8.1 mg, 38% yield) as a yellow solid. MS (ESI) m/z=912.8 [M+H]⁺.

### Example 566. 2-((3-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclobutyl)amino)-N-(6-(dimethylamino)pyridazin-3-yl)-6-(isopropylamino)nicotinamide (CPD-510)

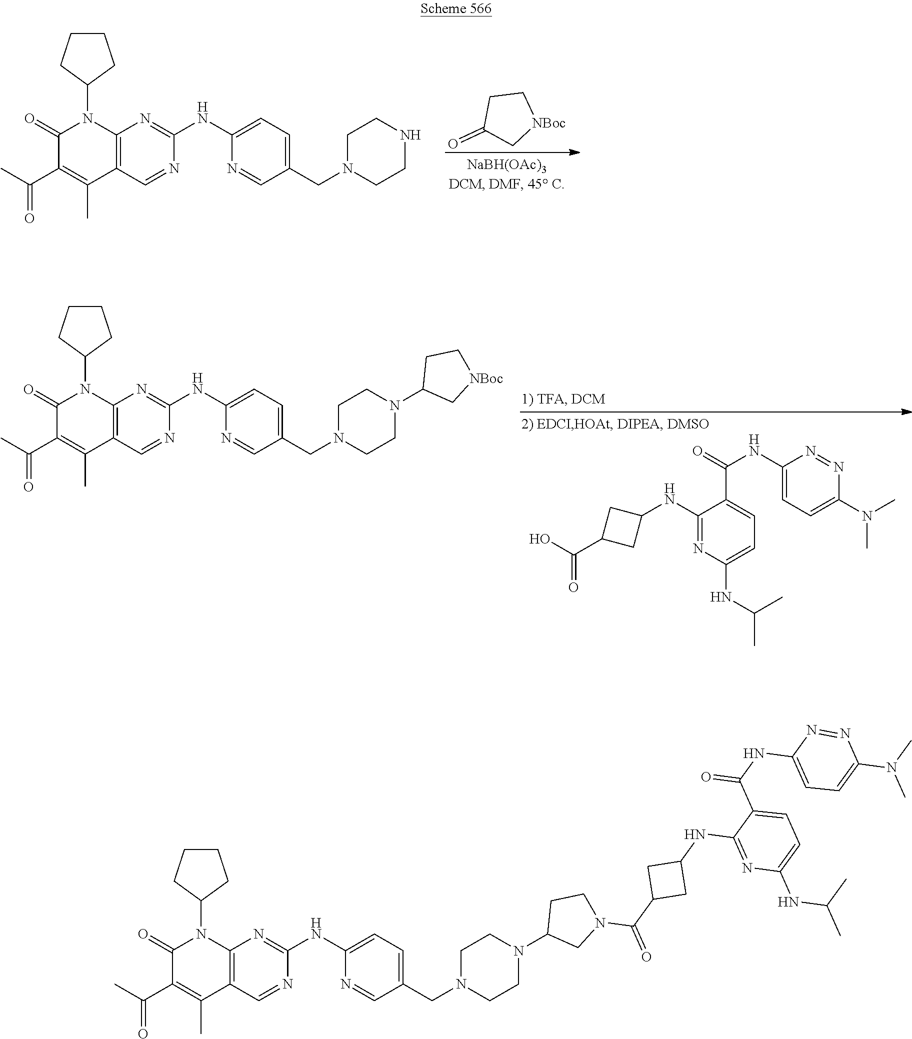 [SMILES: CC(=O)c1c(C)c2cnc(Nc3ccc(CN4CCNCC4)cn3)nc2n(C2CCCC2)c1=O ; CC(C)(C)OC(=O)N1CCC(=O)C1 ; CC(=O)c1c(C)c2cnc(Nc3ccc(CN4CCN(C5CCN(C(=O)OC(C)(C)C)C5)CC4)cn3)nc2n(C2CCCC2)c1=O ; CC(C)Nc1ccc(C(=O)Nc2ccc(N(C)C)nn2)c(NC2CC(C(=O)O)C2)n1 ; CC(=O)c1c(C)c2cnc(Nc3ccc(CN4CCN(C5CCN(C(=O)C6CC(Nc7nc(NC(C)C)ccc7C(=O)Nc7ccc(N(C)C)nn7)C6)C5)CC4)cn3)nc2n(C2CCCC2)c1=O]

### Step 1. Synthesis of tert-butyl 3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carboxylate

To a mixture of 6-acetyl-8-cyclopentyl-5-methyl-2-((5-(piperazin-1-ylmethyl)pyridin-2-yl)amino)pyrido[2,3-d]pyrimidin-7(8H)-one (80 mg, 0.17 mmol) and tert-butyl 3-oxo pyrrolidine-1-carboxylate (96 mg, 0.52 mmol) in dichloromethane (3 mL) and DMF (0.5 mL) was added NaBH(OAc)₃ (183 mg, 0.87 mmol). The reaction mixture was stirred at 45° C. for 16 h. The mixture was quenched with aq. NaHCO₃ solution (8 mL, 5 N) and extracted with dichloromethane (3×10 mL). The combined organic phase was washed with brine, dried over Na₂SO₄, filtered and concentrated. The residue was purified by silica gel chromatography to provide the desired product (45 mg, 41% yield) as a yellow solid. MS (ESI) n/z=632.0 [M+H]⁺.

### Step 2. Synthesis of 6-acetyl-8-cyclopentyl-5-methyl-2-((5-((4-(pyrrolidin-3-yl) piperazin-1-yl)methyl)pyridin-2-yl)amino)pyrido[2,3-d]pyrimidin-7(8H)-one

A mixture of tert-butyl 3-(4-((6-((6-acetyl-8-cyclopentyl-5-methyl-7-oxo-7,8-dihydro pyrido[2,3-d]pyrimidin-2-yl)amino)pyridin-3-yl)methyl)piperazin-1-yl)pyrrolidine-1-carboxylate (45 mg, 71.3 μmol) in dichloromethane (2 mL) and TFA (0.5 mL) was stirred at rt for 3 h. The reaction mixture was concentrated to provide the crude product (30 mg, 79% yield) as a yellow solid. This compound was used directly in the next step without further purification. MS (ESI) n/z=531.9 [M+H]⁺.

### Step 3. Synthesis of 2-((3-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclobutyl)amino)-N-(6-(dimethylamino)pyridazin-3-yl)-6-(isopropylamino) nicotinamide

To a mixture of 3-[[3-[[6-(dimethylamino)pyridazin-3-yl]carbamoyl]-6-(isopropylamino)-2-pyridyl]amino]cyclobutanecarboxylic acid (7.8 mg, 18.8 μmol), EDCI (9.0 mg, 47.1 μmol) and HOAt (2.6 mg, 18.8 μmol) in DMSO (0.5 mL) were added DIPEA (12.2 mg, 94.2 μmol) and 6-acetyl-8-cyclopentyl-5-methyl-2-[[5-[(4-pyrrolidin-3-ylpiperazin-1-yl)methyl]-2-pyridyl]amino]pyrido[2,3-d]pyrimidin-7-one (10 mg, 18.8 mol). The mixture was stirred at rt for 12 h. The mixture was purified by prep-HPLC to provide the title compound (7 mg, 40% yield) as a yellow solid. MS (ESI) n/z=926.8 [M+H]⁺.

### Example 567. 2-((3-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclopentyl)amino)-N-(6-(dimethylamino)pyridazin-3-yl)-6-(isopropylamino)nicotinamide (CPD-511)

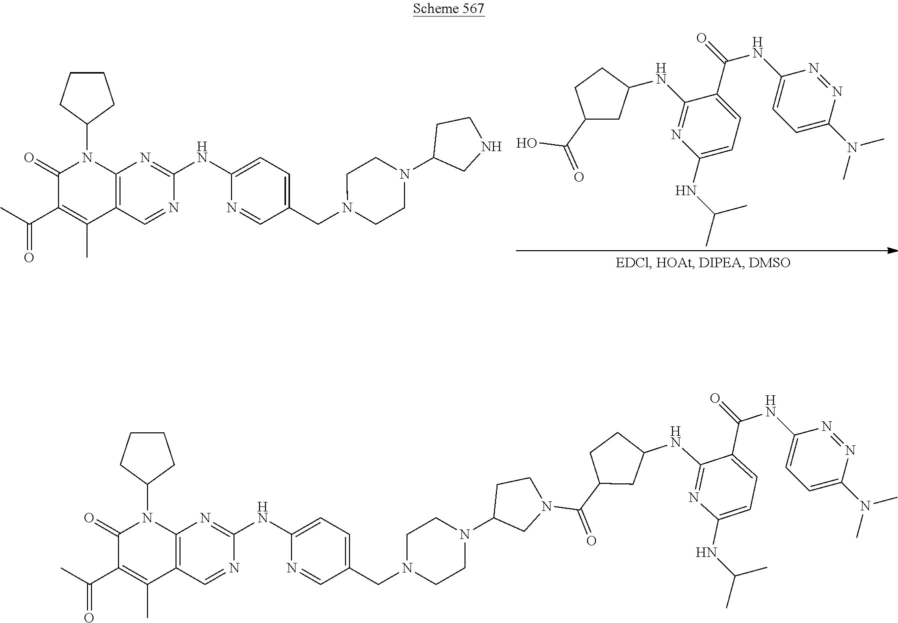

CPD-511 was synthesized following the standard procedure for preparing CPD-143 (11.0 mg, 62% yield) as a yellow solid. MS (ESI) m/z=940.9 [M+H]⁺.

### Example 568. 2-((4-(3-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)pyrrolidine-1-carbonyl)cyclohexyl)amino)-N-(6-(dimethylamino)pyridazin-3-yl)-6-(isopropylamino)nicotinamide (CPD-512)

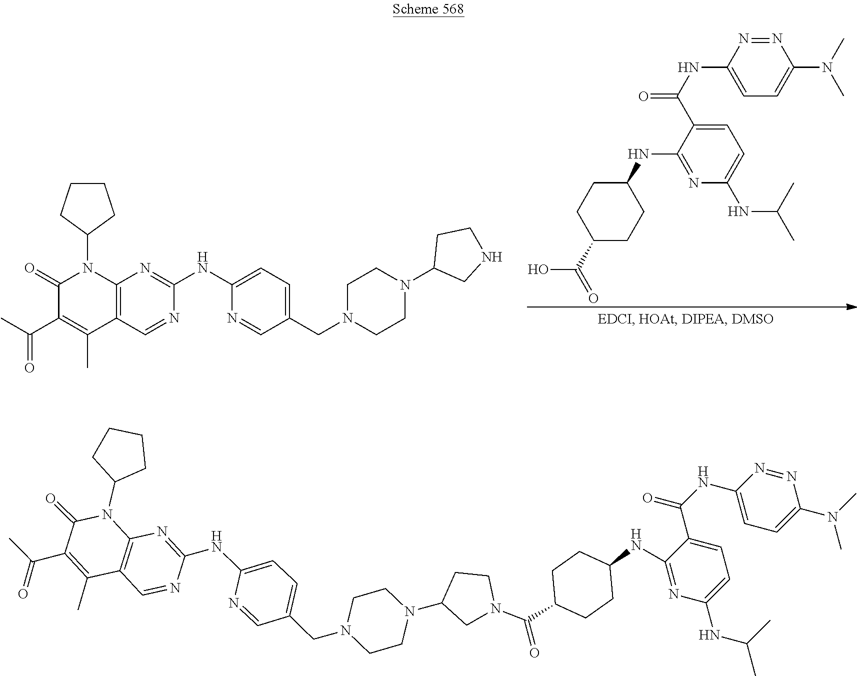 [SMILES: CC(=O)c1c(C)c2cnc(Nc3ccc(CN4CCN(C5CCNC5)CC4)cn3)nc2n(C2CCCC2)c1=O ; CC(C)Nc1ccc(C(=O)Nc2ccc(N(C)C)nn2)c(N[C@H]2CC[C@H](C(=O)O)CC2)n1 ; CC(=O)c1c(C)c2cnc(Nc3ccc(CN4CCN(C5CCN(C(=O)[C@H]6CC[C@H](Nc7nc(NC(C)C)ccc7C(=O)Nc7ccc(N(C)C)nn7)CC6)C5)CC4)cn3)nc2n(C2CCCC2)c1=O]

CPD-512 was synthesized following the standard procedure for preparing CPD-143 (1.9 mg, 10% yield) as a yellow solid. MS (ESI) m/z=954.9 [M+H]⁺.

### Example 569. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)azetidin-3-yl)amino)-N-(6-(dimethylamino)pyridazin-3-yl)-6-(methylamino)nicotinamide (CPD-513)

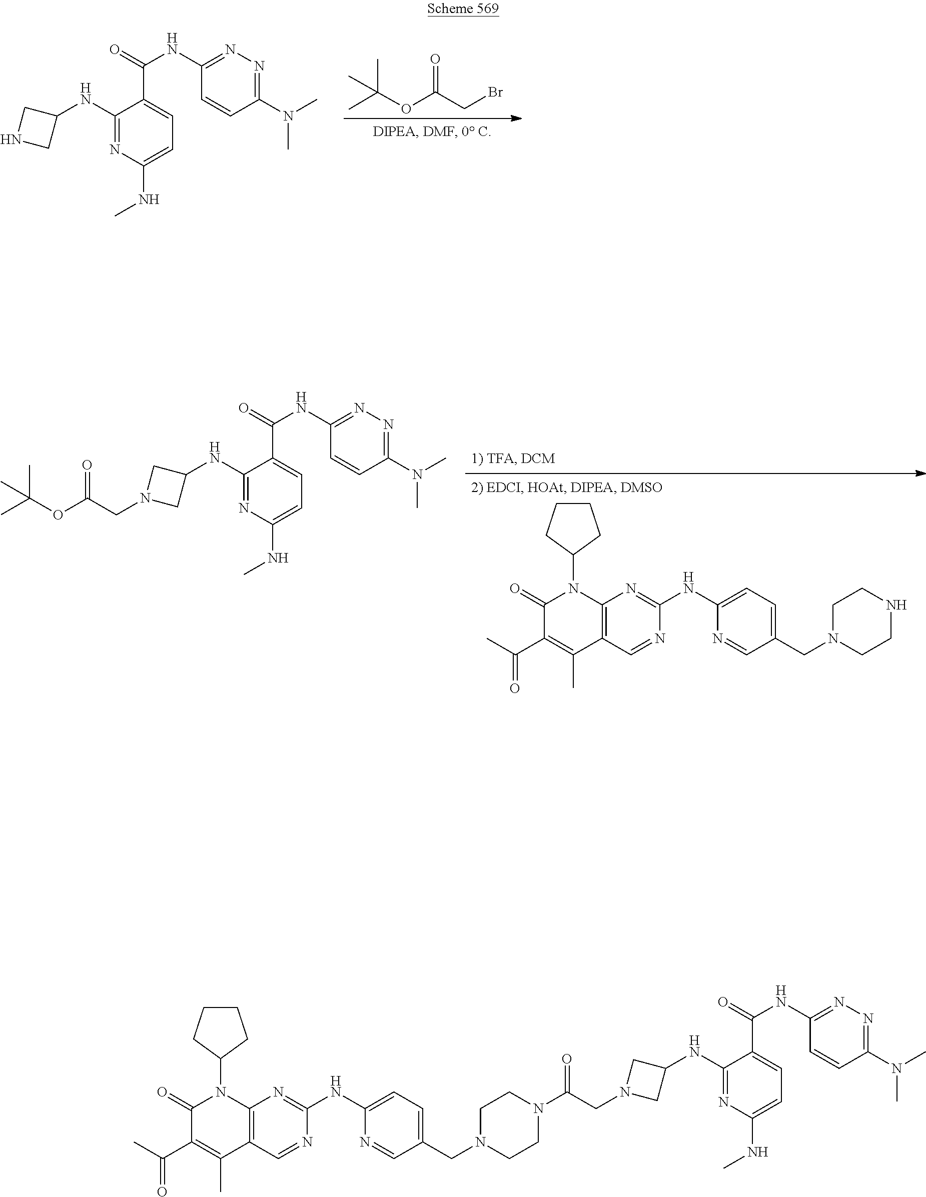

CPD-513 was synthesized following the standard procedures for preparing CPD-418 (6.5 mg, 27% yield over 3 steps) as a yellow solid. MS (ESI) m/z=844.7 [M+H]⁺.

### Example 570. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-6-(isopropylamino)-N-(5-methyl-1-(trifluoromethyl)-1H-pyrazol-3-yl)nicotinamide (CPD-514)

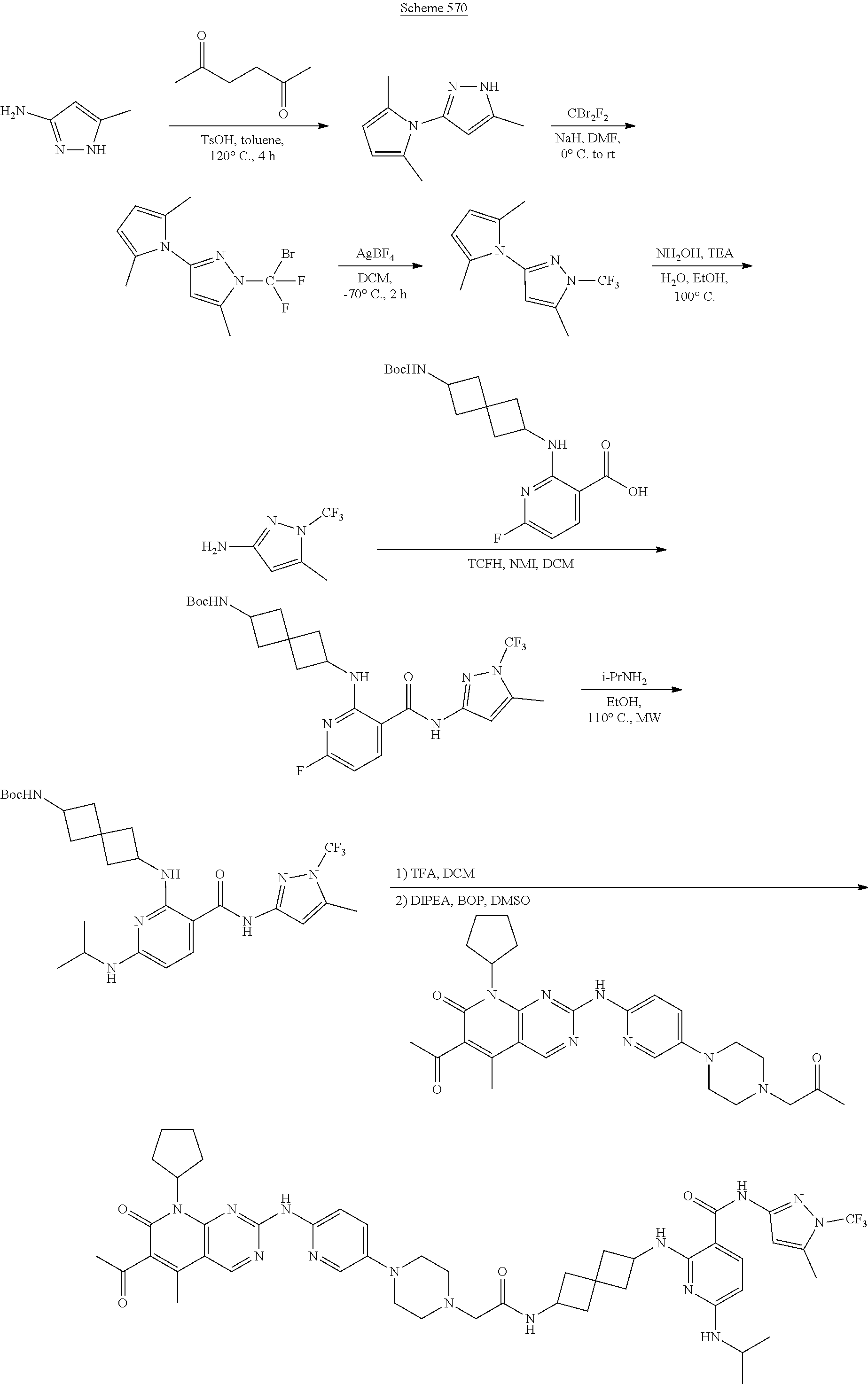

### Step 1. Synthesis of 3-(2,5-dimethylpyrrol-1-yl)-5-methyl-1H-pyrazole

A mixture of 5-methyl-1H-pyrazol-3-amine (5.00 g, 51 mmol), hexane-2,5-dione (5.88 g, 51 mmol, 6.04 and TsOH·H₂O (979 mg, 5.15 mmol) in toluene (20 mL) was degassed and purged with N₂ for 3 times. The mixture was stirred at 120 [symbol] for 4 h under N₂ atmosphere. The reaction mixture was quenched with H₂O (20 mL) and extracted with ethyl acetate (15 mL×3). The combined organic layers were washed with brine (10 mL), dried over Na₂SO₄, filtered, and concentrated under reduced pressure. The residue was purified by column chromatography (SiO₂, petroleum ether/ethyl acetate=10:1 to 5:1) to provide the desired product (3.50 g, 38% yield) as a yellow solid. MS (ESI) m/z=176.2 [M+H]⁺.

### Step 2. Synthesis of 1-[bromo(difluoro)methyl]-3-(2,5-dimethylpyrrol-1-yl)-5-methyl-pyrazole

To a solution of 3-(2,5-dimethylpyrrol-1-yl)-5-methyl-1H-pyrazole (3.50 g, 19.0 mmol) in DMF (40 mL) was added NaH (798 mg, 20.0 mmol) at 0° C. under N₂ atmosphere. The solution was stirred at 0° C. for 10 min. Then dibromo(difluoro)methane (4.61 g, 22.0 mmol) was added at 0° C. The mixture was stirred at 0° C. for 2 h under N₂ atmosphere. The reaction mixture was quenched with H₂O (20 mL) and extracted with ethyl acetate (15 mL×3). The combined organic layers were washed with brine (10 mL), dried over Na₂SO₄, filtered, and concentrated under reduced pressure. The residue was purified by column chromatography (SiO₂, petroleum ether/ethyl acetate=1:0) to provide the desired product (3.50 g, 57% yield) as a yellow oil. MS (ESI) m/z=305.4 [M+H]⁺.

### Step 3. Synthesis of 3-(2,5-dimethylpyrrol-1-yl)-5-methyl-1-(trifluoromethyl)pyrazole

To a stirred solution of 1-[bromo(difluoro)methyl]-3-(2,5-dimethylpyrrol-1-yl)-5-methyl-pyrazole (3.30 g, 10.0 mmol) in dichloromethane (40 mL) was added AgBF₄ (4.22 g, 21.0 mmol) at −78 [symbol] . Then the solution was stirred at 25 [symbol] for 2 h. The reaction mixture was quenched with H₂O (20 mL) and extracted with dichloromethane (20 mL×3). The combined organic layers were washed with brine (15 mL), dried over Na₂SO₄, filtered, and concentrated under reduced pressure to provide the desired product (1.10 g, crude) as a yellow solid. MS (ESI) m/z=244.2 [M+H]⁺.

### Step 4. Synthesis of 5-methyl-1-(trifluoromethyl)pyrazol-3-amine

A mixture of 3-(2,5-dimethylpyrrol-1-yl)-5-methyl-1-(trifluoromethyl)pyrazole (1.00 g, 4.11 mmol), hydroxylamine; hydrochloride (1.43 g, 20.6 mmol) and TEA (1.25 g, 12.33 mmol) in EtOH (20 mL) and H₂O (10 mL) was degassed and purged with N₂ for 3 times in sealed tube. Then the mixture was stirred at 130° C. for 24 h under N₂. The reaction mixture was quenched with H₂O (20 mL) and extracted with ethyl acetate (15 mL×3). The combined organic layers were washed with brine (10 mL), dried over Na₂SO₄, filtered, and concentrated under reduced pressure. The residue was purified by column chromatography (SiO₂, petroleum ether/ethyl acetate=1:1 to 0:1) to provide the desired product (170 mg, 25% yield) as a yellow solid. ¹HNMR (400 MHz, CDCl₃) δ 5.62 (s, 1H), 3.84 (s, 2H), 2.33 (s, 3H). MS (ESI) m/z=166.4 [M+H]⁺.

The remaining steps were performed according to the procedures for preparing CPD-144 to provide the desired product (7.0 mg, 9% yield for 4 steps) as a yellow solid. MS (ESI) m/z=939.8 [M+H]⁺.

### Example 571. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[3.4]octan-6-yl)amino)-N-(6-(dimethylamino)pyridazin-3-yl)-6-(isopropylamino)nicotinamide (CPD-515)

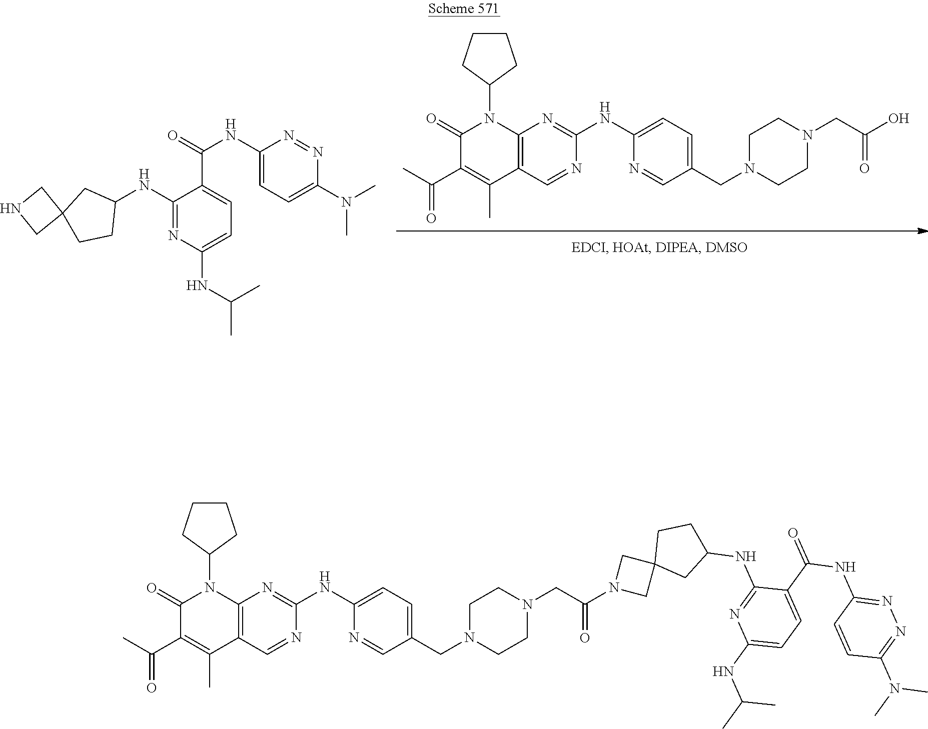

CPD-515 was synthesized following the standard procedure for preparing CPD-143 (3.2 mg, 13% yield) as a yellow solid. MS (ESI) m/z=927.0 [M+H]⁺.

### Example 572. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-methylcyclohexyl)amino)-N-(6-(dimethylamino)pyridazin-3-yl)-6-(isopropylamino)nicotinamide (CPD-516)

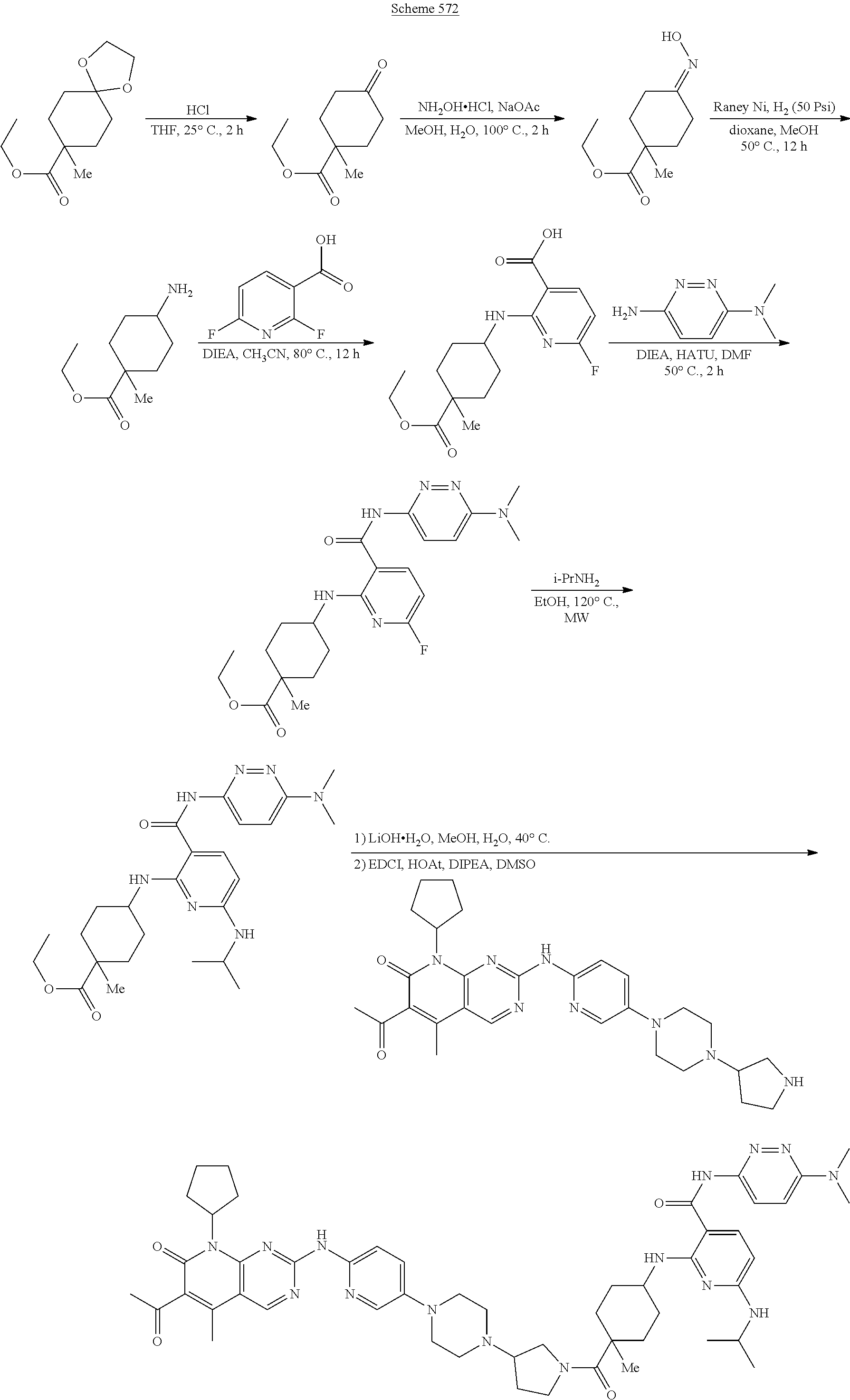

### Step 1. Synthesis of ethyl 1-methyl-4-oxocyclohexane-1-carboxylate

To a solution of ethyl 8-methyl-1,4-dioxaspiro[4.5]decane-8-carboxylate (2.00 g, 8.76 mmol) in THF (20 mL) was added aq. HCl (1 N, 8.75 mL). The mixture was stirred at 25 [symbol] for 2 h. The mixture was adjusted pH to 9~10 with saturated Na₂CO₃ solution and extracted with ethyl acetate (50 mL×3). The combined organic layers were washed with brine (100 mL), dried over Na₂SO₄, filtered, and concentrated to provide the desired product (2.00 g, crude) as a yellow oil. ¹HNMR (400 MHz, CDCl₃) δ 4.25-4.19 (m, 2H), 2.43-2.30 (m, 6H), 1.72-1.64 (m, 2H), 1.31-1.28 (m, 6H).

### Step 2. Synthesis of ethyl 4-(hydroxyimino)-1-methylcyclohexane-1-carboxylate

To a solution of ethyl 1-methyl-4-oxo-cyclohexanecarboxylate (1.00 g, 5.43 mmol) in MeOH (16 mL) and H₂O (4 mL) were added NH₂OH·HCl (420 mg, 6.04 mmol) and NaOAc (490 mg, 5.97 mmol). The mixture was stirred at 100° C. for 2 h. The resulting mixture was concentrated and purified by column chromatography (SiO₂, petroleum ether/ethyl acetate=50:1 to 10:1) to provide the desired product (1.50 g, 69% yield) as a yellow oil. ¹HNMR (400 MHz, CDCl₃) δ 4.19 (q, J=7.2 Hz, 2H), 3.04-2.98 (m, 1H), 2.28-2.20 (m, 6H), 1.46-1.39 (m, 2H), 1.29-1.26 (m, 3H), 1.23 (s, 3H).

### Step 3. Synthesis of ethyl 4-amino-1-methylcyclohexane-1-carboxylate

A mixture of ethyl 4-hydroxyimino-1-methyl-cyclohexanecarboxylate (750 mg, 3.76 mmol) and Raney-Ni (322 mg, 3.76 mmol) in MeOH (1 mL) and dioxane (1 mL) was degassed and purged with N₂ for 3 times. The mixture was stirred at 50 [symbol] for 12 h under H₂ (50 psi). The mixture was filtered through a celite pad and the filtrate evaporated in vacuo to provide the desired product (1.20 g, 86% yield) as a yellow oil. ¹HNMR (400 MHz, CDCl₃) δ 4.17-4.09 (m, 2H), 3.13-2.71 (m, 1H), 1.84-1.70 (m, 4H), 1.47-1.27 (m, 1H), 1.26-1.14 (m, 9H).

### Step 4. Synthesis of 2-((4-(ethoxycarbonyl)-4-methylcyclohexyl)amino)-6-fluoronicotinic acid

To a solution of ethyl 4-amino-1-methyl-cyclohexanecarboxylate (700 mg, 3.78 mmol) and 2,6-difluoropyridine-3-carboxylic acid (700 mg, 4.40 mmol) in CH₃CN (7 mL) was added DIEA (1.47 g, 11.3 mmol). The mixture was stirred at 80° C. for 12 h. The resulting mixture was concentrated and purified by column chromatography (SiO₂, ethyl acetate/AcOH=1:0.001) to provide the desired product (800 mg, 33% yield) as a yellow oil. ¹HNMR (400 MHz, CDCl₃) δ 8.26-8.22 (m, 1H), 6.11-6.07 (m, 1H), 4.15-4.10 (m, 2H), 3.96-3.95 (m, 1H), 2.27-2.01 (m, 1H), 1.95-1.89 (m, 4H), 1.63-1.58 (m, 2H), 1.28-1.25 (m, 3H), 1.19 (s, 3H).

### Step 5. Synthesis of ethyl 4-((3-((6-(dimethylamino)pyridazin-3-yl)carbamoyl)-6-fluoropyridin-2-yl)amino)-1-methylcyclohexane-1-carboxylate

To a solution of 2-[(4-ethoxycarbonyl-4-methyl-cyclohexyl)amino]-6-fluoro-pyridine-3-carboxylic acid (400 mg, 1.23 mmol), HATU (560 mg, 1.47 mmol) and DIEA (480 mg, 3.71 mmol) in DMF (8 mL) was added N³,N³-dimethylpyridazine-3,6-diamine (200 mg, 1.45 mmol). The mixture was stirred at 50° C. for 2 h. The resulting mixture was poured into H₂O (50 mL) and extracted with ethyl acetate (20 mL×3). The combined organic layers were washed with brine (60 mL), dried over Na₂SO₄, filtered and concentrated. The residue was purified by column chromatography (SiO₂, petroleum ether/ethyl acetate=1:1) to provide the desired product (800 mg, 73% yield) as a yellow oil. ¹HNMR (400 MHz, DMSO-d4) (8.44-8.40 (m, 1H), 7.83-7.79 (m, 1H), 7.19-7.15 (m, 1H), 6.28-6.25 (m, 1H), 4.11-4.07 (m, 2H), 4.06-4.03 (m, 1H), 3.08 (s, 6H), 1.91-1.52 (m, 8H), 1.20-1.15 (m, 6H).

The remaining steps were performed according to the procedures for preparing CPD-82 to provide the desired product (3.9 mg, 32% yield) as a yellow solid. MS (ESI) m/z=954.9 [M+H]⁺.

### Example 573. 2-((1-(2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)(methyl)amino)-2-oxoethyl)azetidin-3-yl)amino)-N-(6-(dimethylamino)pyridazin-3-yl)-6-(isopropylamino)nicotinamide (CPD-517)

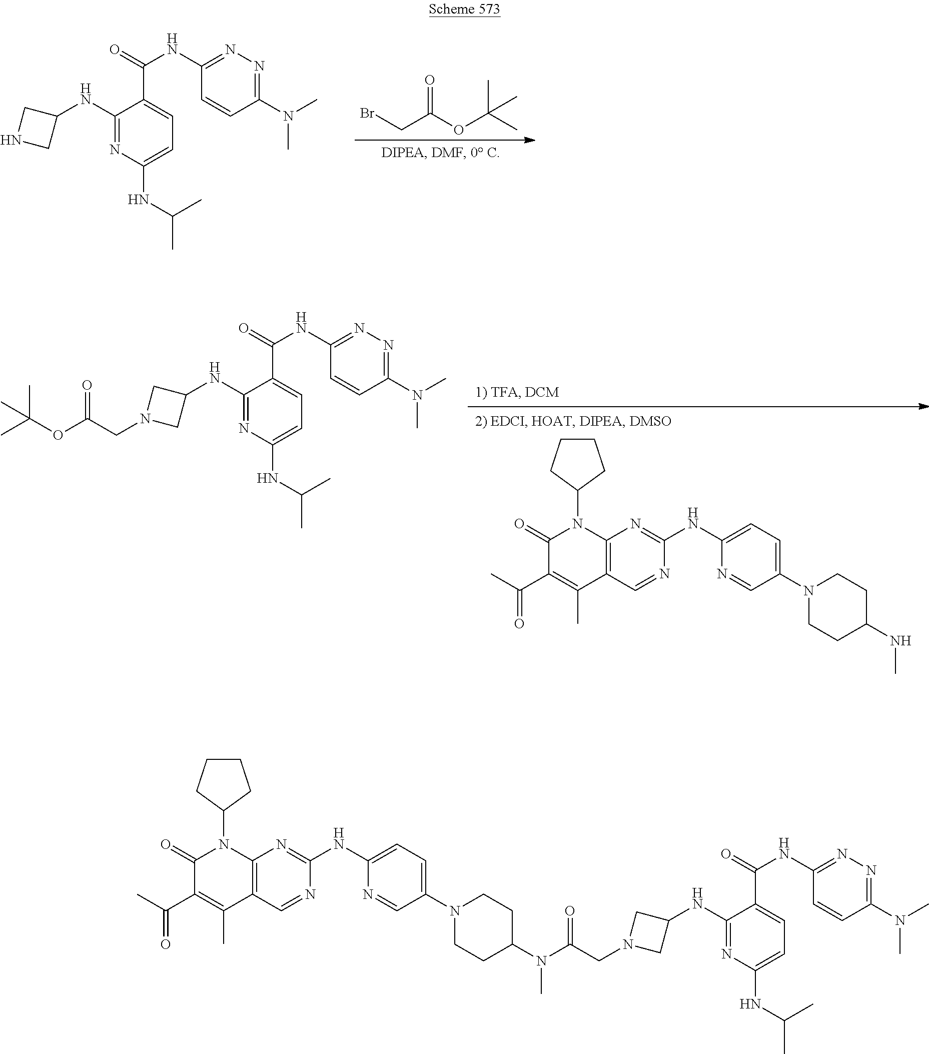

CPD-517 was synthesized following the standard procedures for preparing CPD-418 (1.8 mg, 23% yield over 3 steps) as a yellow solid. MS (ESI) m/z=886.6 [M+H]⁺.

### Example 574. 2-((2-(2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidin-1-yl)-2-oxoethyl)-2-azaspiro[3.3]heptan-6-yl)amino)-N-(6-(dimethylamino)pyridazin-3-yl)-6-(isopropylamino)nicotinamide (CPD-518)

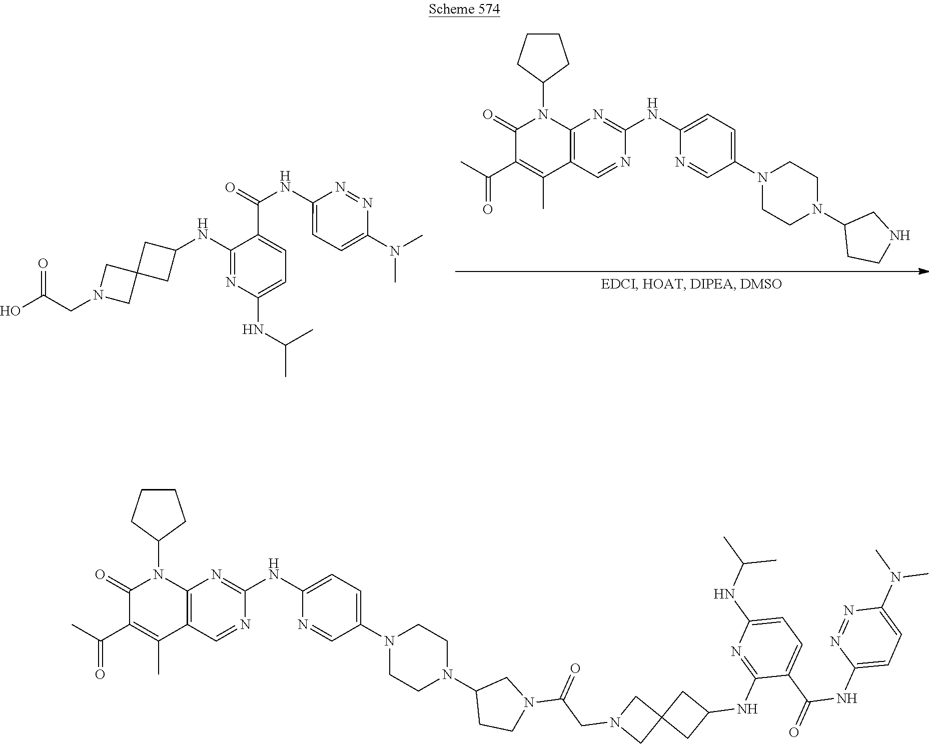

CPD-518 was synthesized following the standard procedures for preparing CPD-143 (7.6 mg, 20% yield) as a yellow solid. MS (ESI) m/z=967.8 [M+H]⁺.

### Example 575. 2-((2-(2-(7-((6-((6-acetyl-8-cyclopentyl-5-methyl-7-oxo-7,8-dihydropyrido[2,3-d]pyrimidin-2-yl)amino)pyridin-3-yl)methyl)-2,7-diazaspiro[4.4]nonan-2-yl)acetyl)-2-azaspiro[3.3]heptan-6-yl)amino)-N-(6-(dimethylamino)pyridazin-3-yl)-6-((methyl-d₃)amino)nicotinamide (CPD-519)

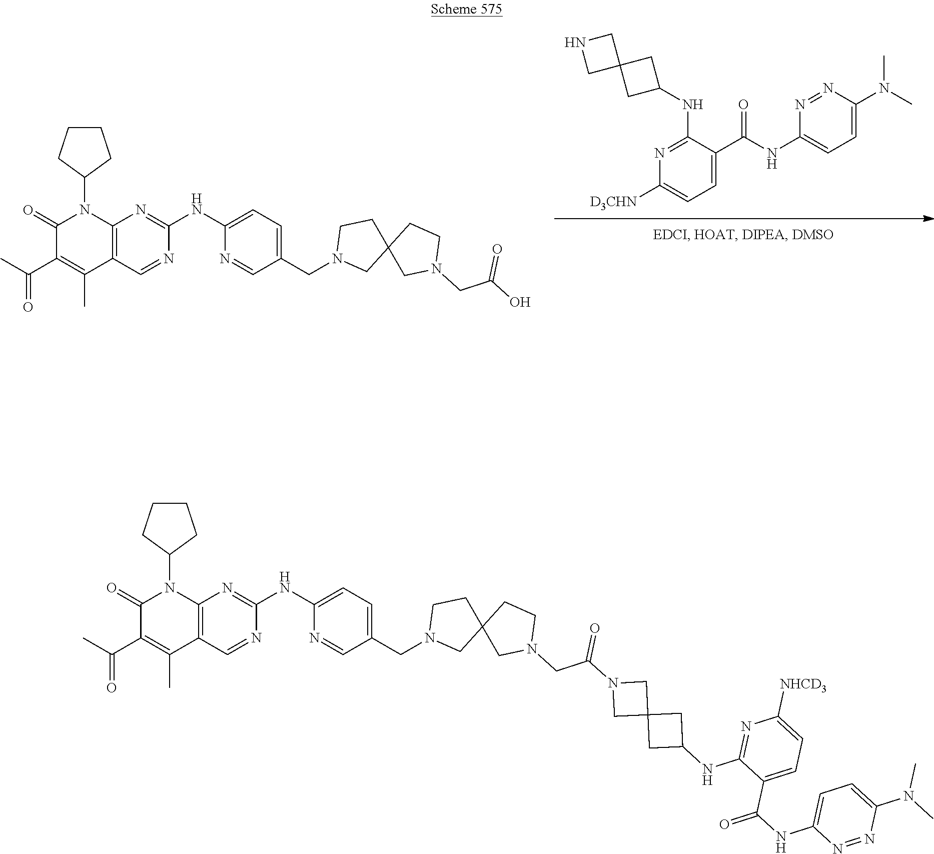

CPD-519 was synthesized following the standard procedure for preparing CPD-143 (7.1 mg, 28% yield) as a yellow solid. MS (ESI) m/z=927.9 [M+H]⁺.

### Example 576. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(5,6-dihydro-4H-pyrrolo[1,2-b]pyrazol-2-yl)-6-(isopropylamino)nicotinamide (CPD-520)

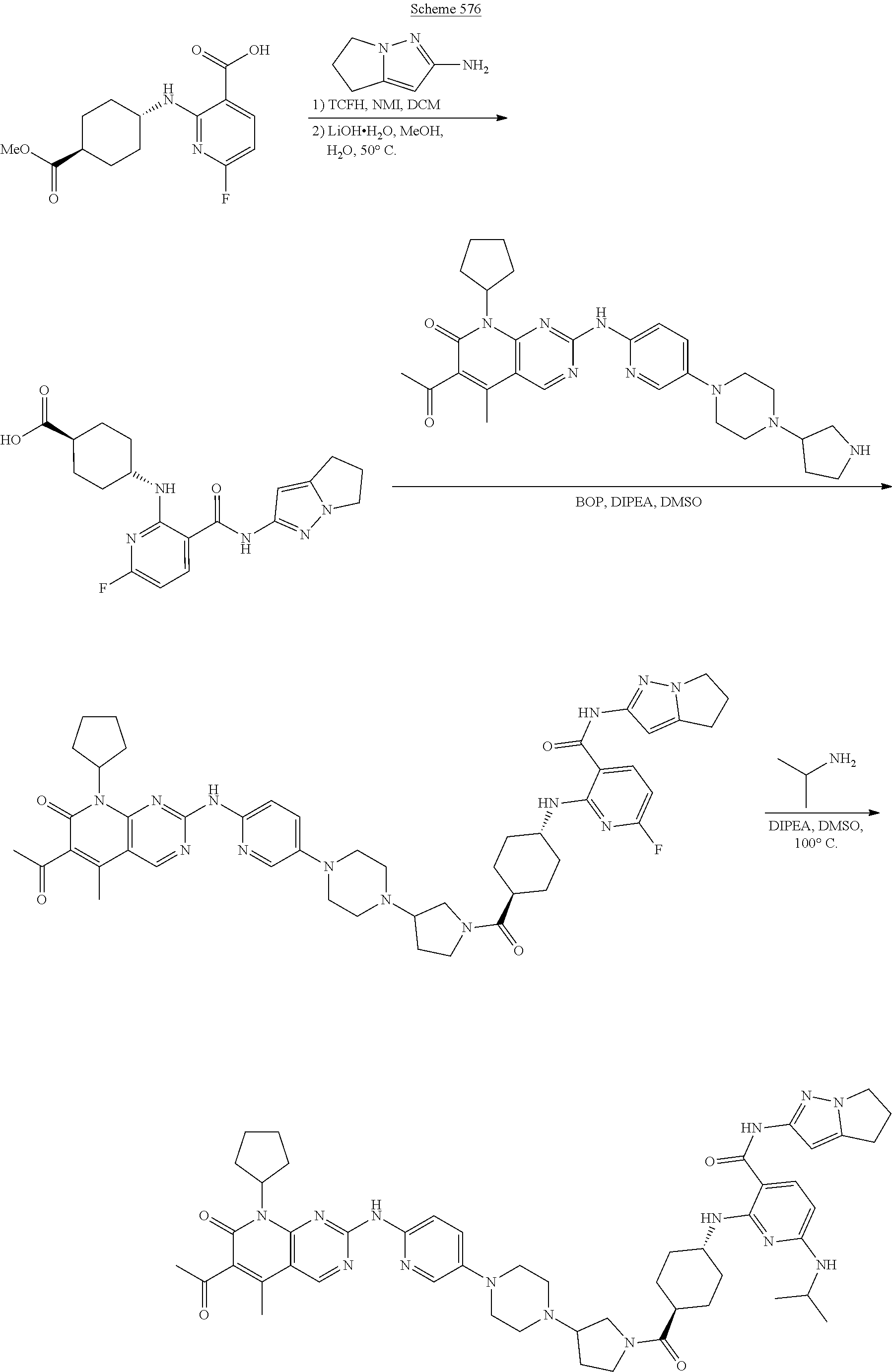

CPD-520 was synthesized following the standard procedures for preparing CPD-477 (7.0 mg, 7% yield over 4 steps) as a yellow solid. MS (ESI) m/z=925.9 [M+H]⁺.

### Example 577. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(5,6-dihydro-4H-pyrrolo[1,2-b]pyrazol-2-yl)-6-((methyl-d3)amino)nicotinamide (CPD-521)

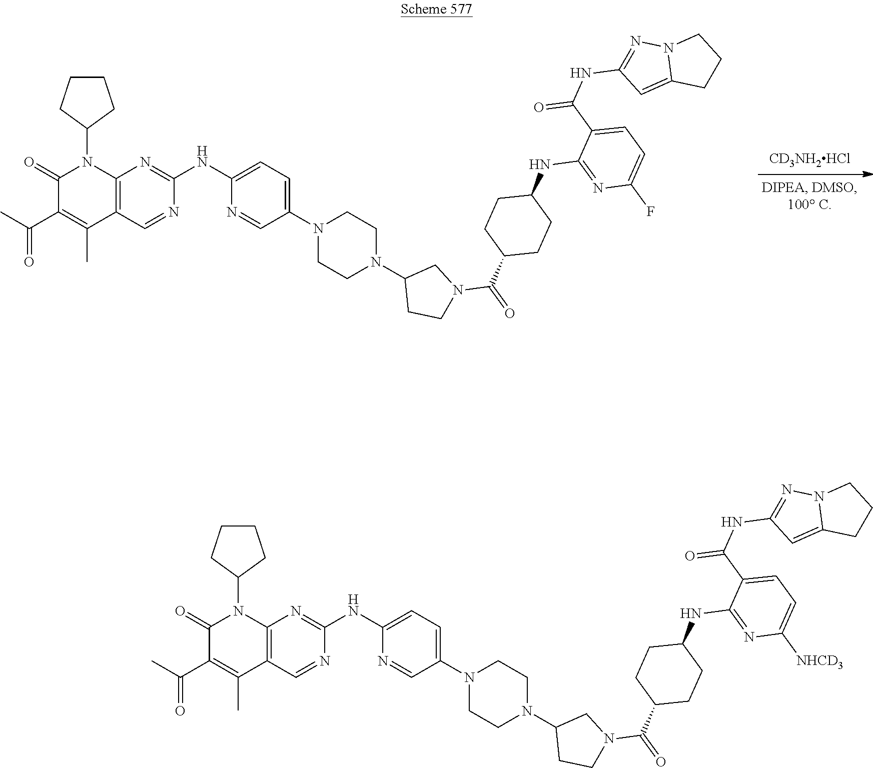

CPD-521 was synthesized following the standard procedure for preparing CPD-477 (10.0 mg, 49% yield) as a yellow solid. MS (ESI) m/z=900.9 [M+H]⁺.

### Example 578. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-(isopropylamino)-N-(6-methoxypyridin-2-yl)nicotinamide (CPD-522)

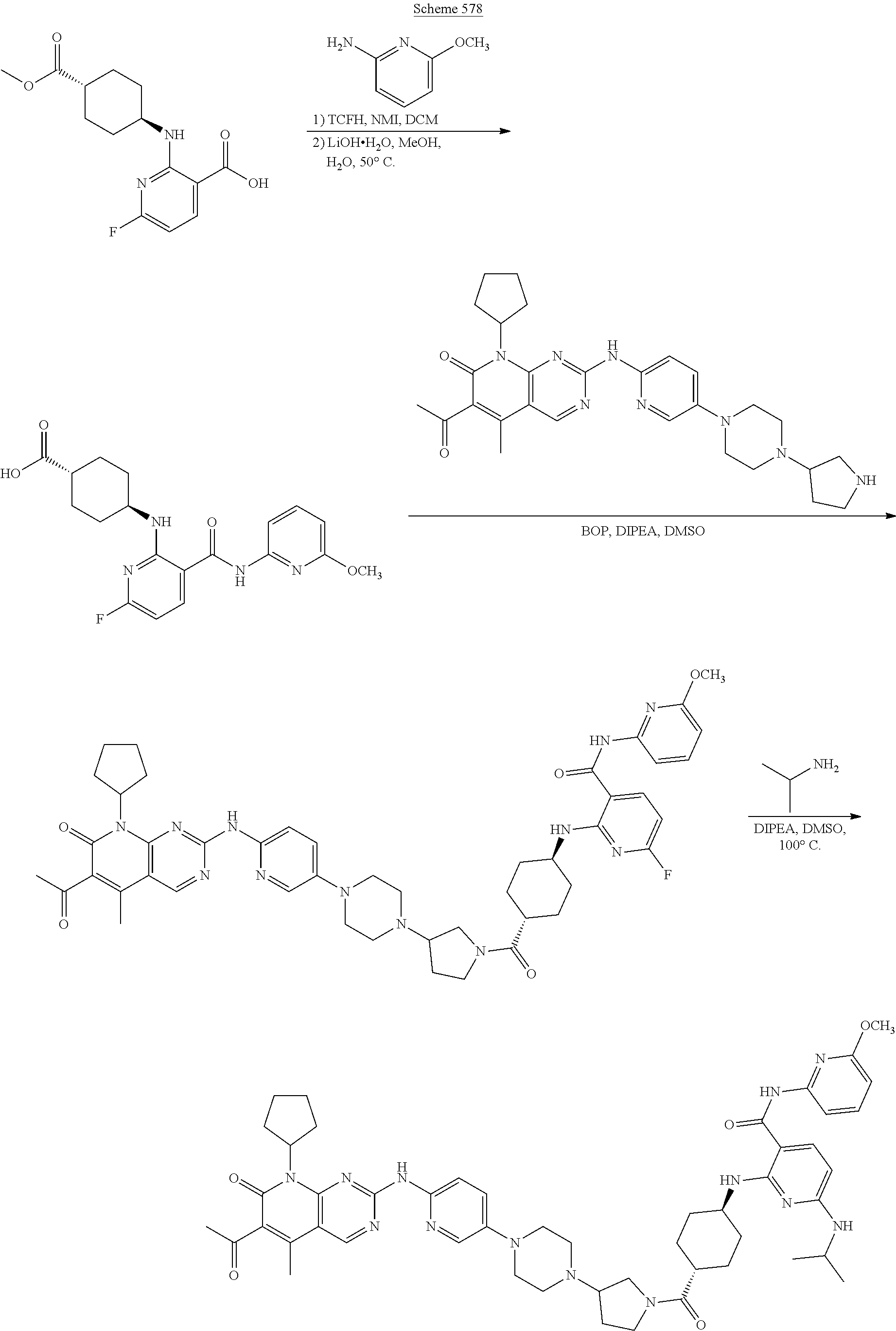

CPD-522 was synthesized following the standard procedures for preparing CPD-477 (8.0 mg, 4% yield over 4 steps) as a yellow solid. MS (ESI) m/z=926.9 [M+H]⁺.

### Example 579. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-methoxypyridin-2-yl)-6-((methyl-d3)amino)nicotinamide (CPD-523)

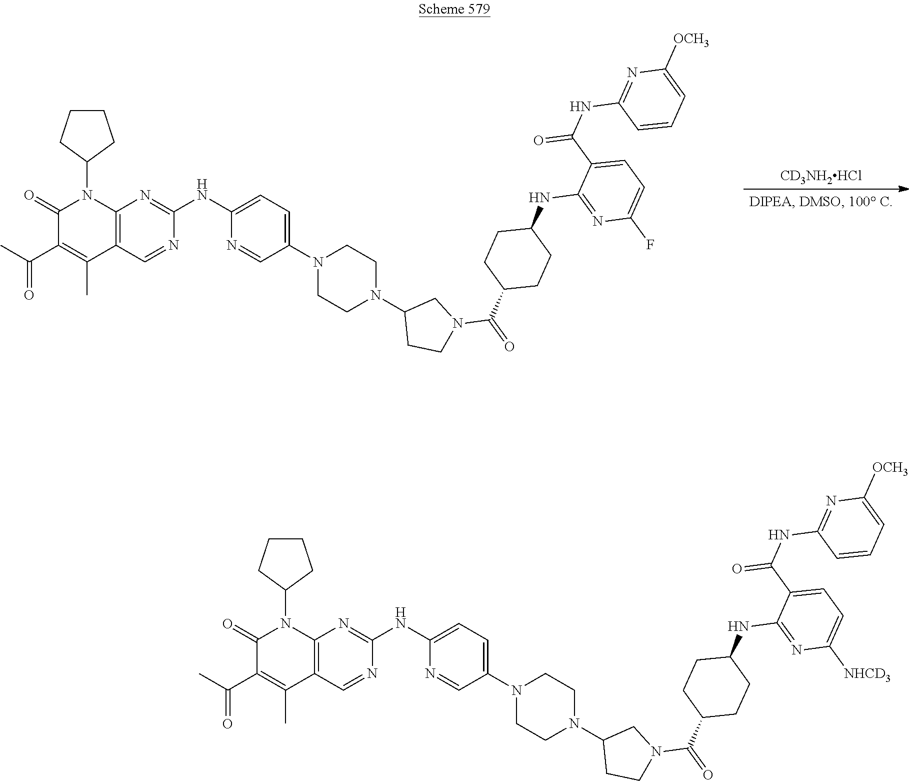

CPD-523 was synthesized following the standard procedure for preparing CPD-477 (8.0 mg, 39% yield) as a yellow solid. MS (ESI) m/z=901.7 [M+H]⁺.

### Example 580. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(5,6-dihydro-4H-pyrrolo[1,2-b]pyrazol-2-yl)-6-((methyl-d₃)amino)nicotinamide (CPD-524)

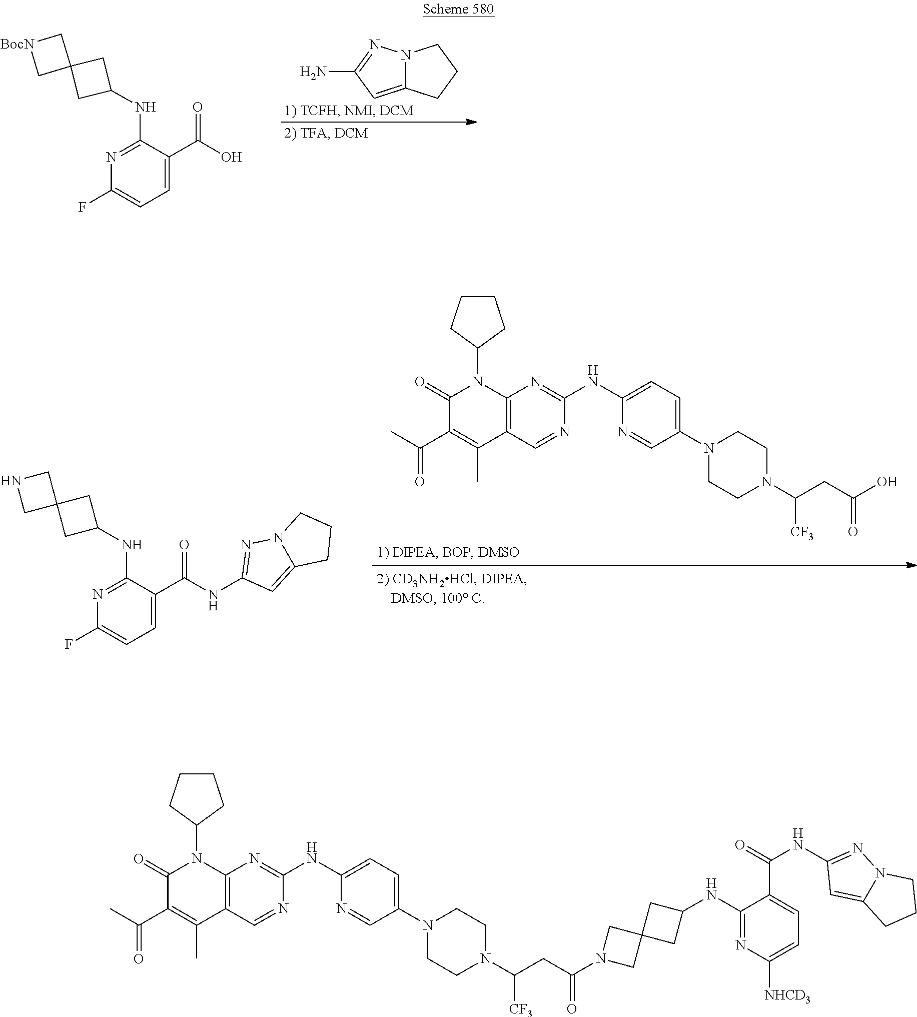

CPD-524 was synthesized following the standard procedures for preparing CPD-477 (5.0 mg, 10% yield over 4 steps) as a yellow solid. MS (ESI) m/z=940.7 [M+H]⁺.

### Example 581. 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)methyl)bicyclo[1.1.1]pentan-1-yl)amino)-N-(6-(dimethylamino)pyridazin-3-yl)-6-(isopropylamino)nicotinamide (CPD-525)

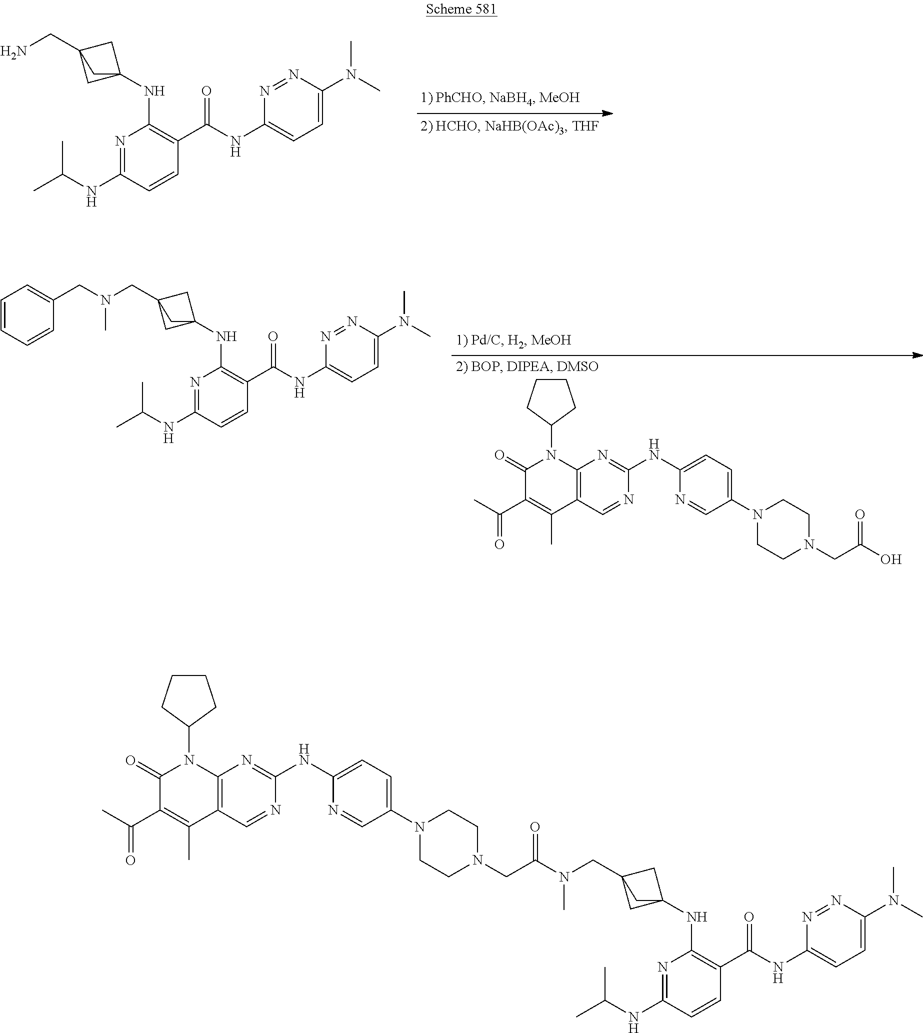 [SMILES: CC(C)Nc1ccc(C(=O)Nc2ccc(N(C)C)nn2)c(NC23CC(CN)(C2)C3)n1 ; CC(C)Nc1ccc(C(=O)Nc2ccc(N(C)C)nn2)c(NC23CC(CN(C)Cc4ccccc4)(C2)C3)n1 ; CC(=O)c1c(C)c2cnc(Nc3ccc(N4CCN(CC(=O)O)CC4)cn3)nc2n(C2CCCC2)c1=O ; CC(=O)c1c(C)c2cnc(Nc3ccc(N4CCN(CC(=O)N(C)CC56CC(Nc7nc(NC(C)C)ccc7C(=O)Nc7ccc(N(C)C)nn7)(C5)C6)CC4)cn3)nc2n(C2CCCC2)c1=O]

### Step 1. Synthesis of 2-((3-((benzylamino)methyl)bicyclo[1.1.1]pentan-1-yl)amino)-N-(6-(dimethylamino)pyridazin-3-yl)-6-(isopropylamino)nicotinamide

To a solution of 2-((3-(aminomethyl)bicyclo[1.1.1]pentan-1-yl)amino)-N-(6-(dimethylamino)pyridazin-3-yl)-6-(isopropylamino)nicotinamide (40 mg, 0.1 mmol) in MeOH (5 mL) were added benzaldehyde (103 mg, 0.97 mmol) and NaBH₄ (37 mg, 0.97 mmol) at 0° C. The mixture was stirred at rt for 4 h. The reaction mixture was poured into water (10 mL) and extracted with ethyl acetate (3×10 mL). The combined organic layers were washed with saturated brine (10 mL), dried over anhydrous sodium sulfate, filtered, and concentrated. The residue was purified by prep-TLC to give the desired product (25 mg, 51% yield) as a light-yellow solid. MS (ESI) m/z=501.6 [M+H]⁺.

### Step 2. Synthesis of 2-((3-((benzyl(methyl)amino)methyl)bicyclo[1.1.1]pentan-1-yl)amino)-N-(6-(dimethylamino)pyridazin-3-yl)-6-(isopropylamino)nicotinamide

To a solution of 2-((3-((benzylamino)methyl)bicyclo[1.1.1]pentan-1-yl)amino)-N-(6-(dimethylamino)pyridazin-3-yl)-6-(isopropylamino)nicotinamide (25 mg, 0.05 mmol) in THF (5 mL) were added formalin (15 mg, 0.50 mmol) and NaBH(OAc)₃ (32 mg, 0.15 mmol). The mixture was stirred at rt for 2 h. The reaction mixture was poured into water (10 mL) and extracted with ethyl acetate (3×10 mL). The combined organic layers were washed with saturated brine (10 mL), dried over anhydrous sodium sulfate, filtered, and concentrated. The residue was purified by pre-TLC to give the desired product (20 mg, 78% yield) as a light-yellow solid. MS (ESI) m/z=515.6 [M+H]⁺.

### Step 3. Synthesis of N-(6-(dimethylamino)pyridazin-3-yl)-6-(isopropylamino)-2-((3-((methylamino)methyl)bicyclo[1.1.1]pentan-1-yl)amino)nicotinamide

To a solution of 2-((3-((benzyl(methyl)amino)methyl)bicyclo[1.1.1]pentan-1-yl)amino)-N-(6-(dimethylamino)pyridazin-3-yl)-6-(isopropylamino)nicotinamide (20 mg, 0.04 mmol) in MeOH (5 mL) was added 10% Pd/C (15 mg). The mixture was stirred at rt under hydrogen atmosphere for 3 h. The mixture was filtered and concentrated to give the desired product (5 mg, 30% yield) as a white solid. MS (ESI) m/z=425.4 [M+H]⁺.

### Step 4. Synthesis of 2-((3-((2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-N-methylacetamido)methyl)bicyclo[1.1.1]pentan-1-yl)amino)-N-(6-(dimethylamino)pyridazin-3-yl)-6-(isopropylamino)nicotinamide

To a solution of 2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetic acid (10 mg, 0.02 mmol) in DMSO (3 mL) were added N-(6-(dimethylamino)pyridazin-3-yl)-6-(isopropylamino)-2-((3-((methylamino)methyl)bicyclo[1.1.1]pentan-1-yl)amino)nicotinamide (13 mg, 0.02 mmol), DIPEA (13 mg, 0.10 mmol) and BOP (18 mg, 0.04 mmol). The mixture was stirred at rt for 2 h. The mixture was purified by reverse-phase chromatography to give the desired product (2 mg, 11% yield) as a yellow solid. MS(ESI) m/z=912.7 [M+H]⁺.

### Example 582. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-ethylbenzamide (CPD-526)

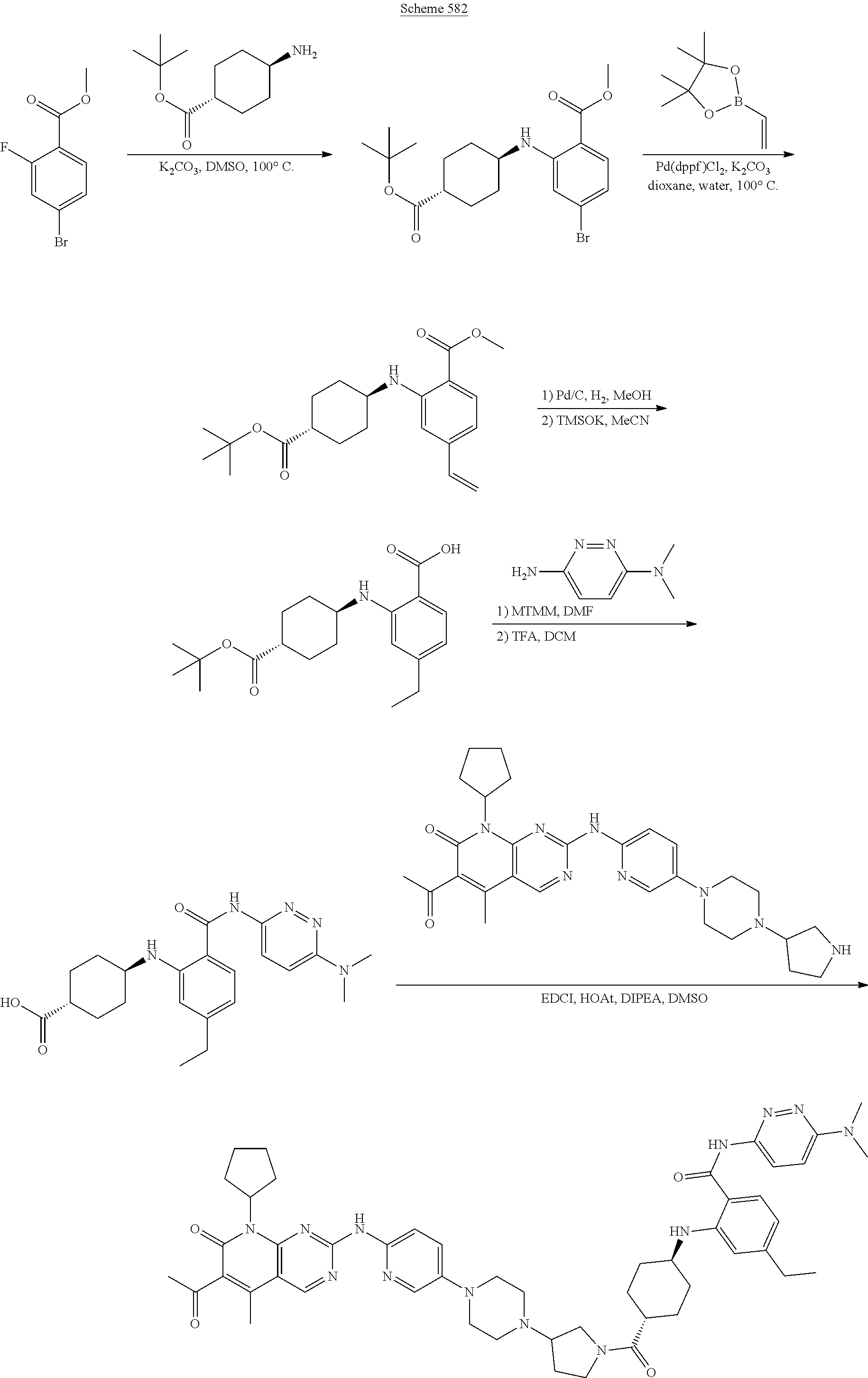

### Step 1. Synthesis of methyl 4-bromo-2-((trans-4-(tert-butoxycarbonyl)cyclohexyl)amino)benzoate

To a solution of methyl 4-bromo-2-fluorobenzoate (300 mg, 1.29 mmol) in DMSO (5 mL) were added tert-butyl trans-4-aminocyclohexane-1-carboxylate (307.87 mg, 1.54 mmol) and K₂CO₃ (266.88 mg, 1.93 mmol) at rt. The reaction mixture was stirred at 100° C. for 16 h. After cooling down to rt, the mixture was poured into water (100 mL) and extracted with EtOAc (3×50 mL). The combined organic layers were washed with saturated brine (75 mL), dried over anhydrous sodium sulfate, filtered, and evaporated under reduced pressure. The resulting residue was purified by reverse-phase chromatography to give the desired product (200 mg, 38% yield) as a pale-white solid. MS (ESI) m/z=412.6 [M+H]⁺.

### Step 2. Synthesis of methyl 2-((trans-4-(tert-butoxycarbonyl)cyclohexyl)amino)-4-vinylbenzoate

To a solution of methyl 4-bromo-2-((trans-4-(tert-butoxycarbonyl)cyclohexyl)amino)benzoate (80 mg, 216.08 μmol) in dioxane (5 mL) and water (I mL) were added 4,4,5,5-tetramethyl-2-vinyl-1,3,2-dioxaborolane (66.56 mg, 432.16 μmol), Pd(dppf)Cl₂ (158.10 mg, 216.08 μmol) and K₂CO₃ (29.86 mg, 216.08 μmol) at rt. The mixture was stirred at 100 [symbol] under inert atmosphere for 16 h. The reaction mixture was cooled to rt, filtered and concentrated. The resulting residue was purified by silica gel chromatography to give the desired product (58 mg, 75% yield) as a pale-white solid. MS (ESI) m/z=360.8 [M+H]⁺.

### Step 3. Synthesis of methyl 2-((trans-4-(tert-butoxycarbonyl)cyclohexyl)amino)-4-ethylbenzoate

To a solution of methyl 2-((trans-4-(tert-butoxycarbonyl)cyclohexyl)amino)-4-vinylbenzoate) in MeOH (5 mL) was added 5% Pd/C (10 mg). The reaction mixture was stirred at rt under hydrogen atmosphere for 2 h. The reaction mixture was filtered and concentrated under reduced pressure. The residue was purified by silica gel chromatography to give the desired product (40 mg, 69% yield) as a white solid. MS (ESI) m/z=363.0 [M+H]⁺.

### Step 4. Synthesis of 2-((trans-4-(tert-butoxycarbonyl)cyclohexyl)amino)-4-ethylbenzoic acid

To a solution of methyl 2-((trans-4-(tert-butoxycarbonyl)cyclohexyl)amino)-4-ethylbenzoate (38 mg, 105.13 mol) in MeCN (5 mL) was added TMSOK (53.94 mg, 420.50 μmol) at rt. The reaction mixture was stirred at rt for 16 h. The mixture was poured into water (30 mL) and extracted with dichloromethane (3×15 mL). The combined organic layers were washed with saturated brine (20 mL), dried over anhydrous sodium sulfate, filtered, and evaporated under reduced pressure. The resulting residue was purified by reverse-phase chromatography to give the desired product (30 mg, 82% yield) as a pale-white solid. MS (ESI) m/z=348.8 [M+H]⁺.

### Step 5. Synthesis of tert-butyl trans-4-((2-((6-(dimethylamino)pyridazin-3-yl)carbamoyl)-5-ethylphenyl)amino)cyclohexane-1-carboxylate

To a solution of 2-((trans-4-(tert-butoxycarbonyl)cyclohexyl)amino)-4-ethylbenzoic acid (30 mg, 86.34 μmol) in DMF (2 mL) were added N³,N³-dimethylpyridazine-3,6-diamine (14.32 mg, 103.61 μmol) and MTMM (28.33 mg, 86.34 μmol) at rt. The reaction mixture was stirred at rt for 16 h. The mixture was poured into water (20 mL) and extracted with dichloromethane (3×10 mL). The combined organic layers were washed with saturated brine (15 mL), dried over anhydrous sodium sulfate, filtered, and evaporated under reduced pressure. The resulting residue was purified by reverse-phase chromatography to give the desired product (15 mg, 37% yield) as a white solid. MS (ESI) m/z=468.8 [M+H]⁺.

### Step 6. Synthesis of trans-4-((2-((6-(dimethylamino)pyridazin-3-yl)carbamoyl)-5-ethylphenyl)amino)cyclohexane-1-carboxylic acid

A solution of tert-butyl trans-4-((2-((6-(dimethylamino)pyridazin-3-yl)carbamoyl)-5-ethylphenyl)amino)cyclohexane-1-carboxylate (15 mg, 32.08 μmol) in dichloromethane (2 mL) and TFA (1 mL) was stirred at rt for 2 h. The reaction mixture was concentrated under reduced pressure to give the crude product (7 mg, 53% yield) as an oil, which was used directly in the next step without further purification. MS(ESI) m/z=412.7 [M+H]⁺.

### Step 7. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-ethylbenzamide

To a mixture of 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (8.79 mg, 17.01 μmol), EDCI (4.89 mg, 25.52 μmol) and HOAt (3.47 mg, 25.52 μmol) in DMSO (1 mL) were added DIPEA (10.99 mg, 85.06 mol) and trans-4-((2-((6-(dimethylamino)pyridazin-3-yl)carbamoyl)-5-ethylphenyl)amino)cyclohexane-1-carboxylic acid (7 mg, 17.01 μmol). The mixture was stirred at rt for 16 h, before it was purified by prep-HPLC to provide the desired product (0.8 mg, 5% yield) as a yellow solid. MS (ESI) m/z=912.0 [M+H]⁺.

### Example 583. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-(isopropylamino)benzamide (CPD-527)

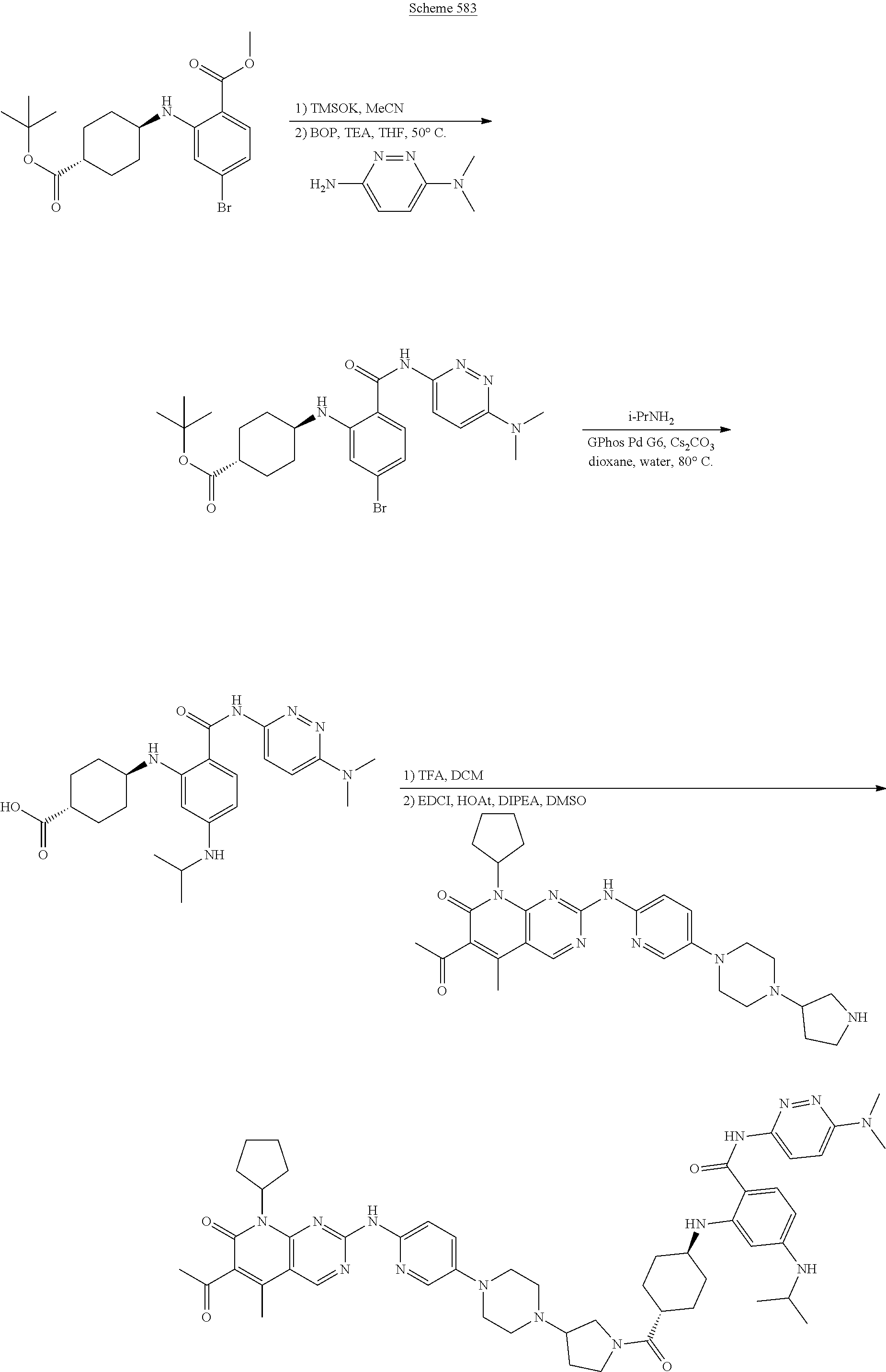

### Step 1. Synthesis of 4-bromo-2-((trans-4-(tert-butoxycarbonyl)cyclohexyl)amino)benzoic acid

To a solution of methyl 4-bromo-2-((trans-4-(tert-butoxycarbonyl)cyclohexyl)amino)benzoate (680 mg, 1.65 mmol) in MeCN (10 mL) was added TMSOK (846.29 mg, 6.60 mmol) at rt. The mixture was stirred at rt for 16 h. The reaction mixture was quenched with water (50 mL), acidified with aq. HCl (1 M), and extracted with dichloromethane (3×40 mL). The combined organic layers were washed with saturated brine (60 mL), dried over anhydrous sodium sulfate, filtered, and concentrated. The resulting residue was purified by reverse-phase chromatography to give the desired product (600 mg, 91% yield) as a pale-white solid. MS (ESI) m/z=398.6 [M+H]⁺.

### Step 2. Synthesis of tert-butyl trans-4-((5-bromo-2-((6-(dimethylamino)pyridazin-3-yl)carbamoyl)phenyl)amino)cyclohexane-1-carboxylate

To a solution of 4-bromo-2-((trans-4-(tert-butoxycarbonyl)cyclohexyl)amino)benzoic acid (100 mg, 251.07 μmol) in THF (5 mL) were added N³,N³-dimethylpyridazine-3,6-diamine (41.63 mg, 301.29 μmol), BOP (222.09 mg, 502.15 μmol) and TEA (127.03 mg, 1.26 mmol). The mixture was stirred at 50° C. for 16 h. The reaction mixture was quenched with water (30 mL) and extracted with dichloromethane (3×20 mL). The combined organic layers were washed with saturated brine (30 mL), dried over anhydrous sodium sulfate, filtered, and concentrated. The resulting residue was purified by reverse-phase chromatography to give the desired product (70 mg, 54% yield) as a white solid. MS (ESI) m/z=518.7 [M+H]⁺.

### Step 3. Synthesis of tert-butyl trans-4-((2-((6-(dimethylamino)pyridazin-3-yl)carbamoyl)-5-(isopropylamino)phenyl)amino)cyclohexane-1-carboxylate

To a solution of tert-butyl trans-4-((5-bromo-2-((6-(dimethylamino)pyridazin-3-yl)carbamoyl)phenyl)amino)cyclohexane-1-carboxylate (70 mg, 135.02 μmol) in dioxane (5 mL) were added isopropylamine (79.81 mg, 1.35 mmol), GPhos Pd G6 (12.78 mg, 13.50 μmol) and Cs₂CO₃ (43.99 mg, 135.02 μmol) at rt. The mixture was stirred at 80 [symbol] under inert atmosphere for 16 h. The reaction mixture was cooled to rt, quenched with water (20 mL), and extracted with dichloromethane (3×20 mL). The combined organic layers were washed with saturated brine (30 mL), dried over anhydrous sodium sulfate, filtered, and concentrated. The resulting residue was purified by silica gel chromatography to give the desired product (25 mg, 37% yield) as a yellow solid. MS (ESI) m/z=497.6 [M+H]⁺.

### Step 4. Synthesis of trans-4-((2-((6-(dimethylamino)pyridazin-3-yl)carbamoyl)-5-(isopropylamino)phenyl)amino)cyclohexane-1-carboxylic acid

A solution of tert-butyl trans-4-((2-((6-(dimethylamino)pyridazin-3-yl)carbamoyl)-5-(isopropylamino)phenyl)amino)cyclohexane-1-carboxylate (25 mg, 50.34 μmol) in dichloromethane (2 mL) and TFA (1 mL) was stirred at rt for 2 h. The reaction mixture was concentrated to give the crude product (15 mg, 68% yield) as a colorless oil, which was used directly in the next step without further purification. MS(ESI) m/z=441.5 [M+H]⁺.

### Step 5. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-(isopropylamino)benzamide

To a mixture of trans-4-((2-((6-(dimethylamino)pyridazin-3-yl)carbamoyl)-5-(isopropylamino)phenyl)amino)cyclohexane-1-carboxylic acid (15 mg, 34.05 μmol), EDCI (9.79 mg, 51.07 μmol) and HOAt (6.95 mg, 51.07 μmol) in DMSO (1 mL) were added DIPEA (22.00 mg, 170.25 μmol) and 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (17.59 mg, 34.05 μmol). The mixture was stirred at rt for 16 h, before it was purified by prep-HPLC to provide the desired product (1.0 mg, 3% yield) as a yellow solid. MS (ESI) m/z=941.1 [M+H]⁺.

### Example 584. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-isopropylbenzamide (CPD-528)

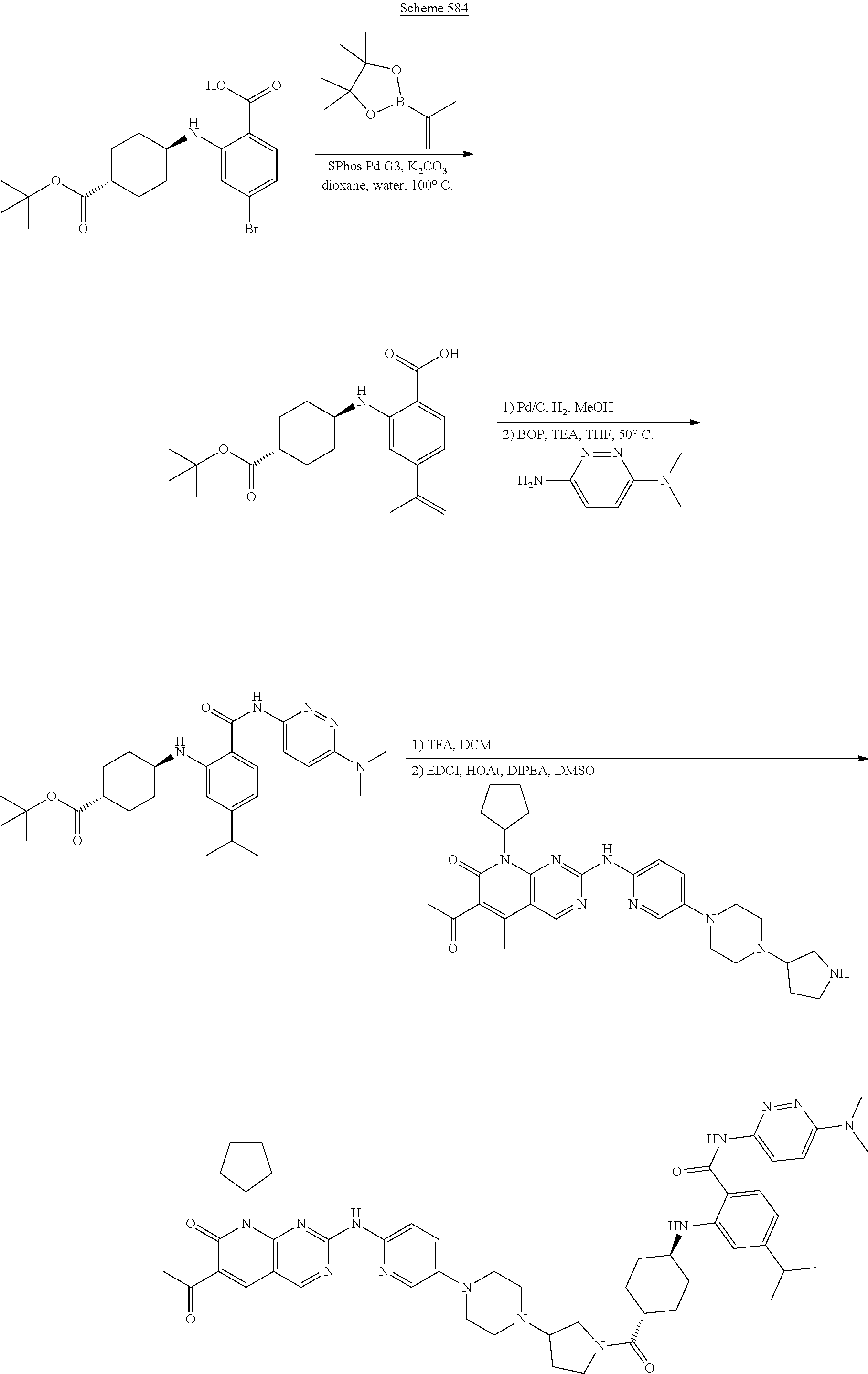

### Step 1. Synthesis of 2-((trans-4-(tert-butoxycarbonyl)cyclohexyl)amino)-4-(prop-1-en-2-yl)benzoic acid

To a solution of 4-bromo-2-((trans-4-(tert-butoxycarbonyl)cyclohexyl)amino)benzoic acid (100 mg, 251.07 μmol) in dioxane (4 mL) and water (1 mL) were added 4,4,5,5-tetramethyl-2-(prop-1-en-2-yl)-1,3,2-dioxaborolane (84.38 mg, 502.15 μmol), SPhos Pd G3 (19.59 mg, 25.11 μmol) and Cs₂CO₃ (81.82 mg, 251.07 μmol) at rt. The mixture was stirred at 100 [symbol] under inert atmosphere for 2 h. The reaction mixture was filtered and concentrated. The resulting residue was purified by silica gel chromatography to give the desired product (81 mg, 90% yield) as a pale-white solid. MS (ESI) m/z=360.8 [M+H]⁺.

### Step 2. Synthesis of 2-((trans-4-(tert-butoxycarbonyl)cyclohexyl)amino)-4-isopropylbenzoic acid

To a solution of 2-((trans-4-(tert-butoxycarbonyl)cyclohexyl)amino)-4-(prop-1-en-2-yl)benzoic acid (81 mg, 225.34 μmol) in MeOH (5 mL) was added 5% Pd/C (10 mg). The mixture was stirred at rt for 2 h under hydrogen atmosphere. The reaction mixture was filtered and concentrated. The resulting residue was purified by silica gel chromatography to give the desired product (55 mg, 68% yield). MS (ESI) m/z=362.5 [M+H]⁺.

### Step 3. Synthesis of tert-butyl trans-4-((2-((6-(dimethylamino)pyridazin-3-yl)carbamoyl)-5-isopropylphenyl)amino)cyclohexane-1-carboxylate

To a solution of 2-((trans-4-(tert-butoxycarbonyl)cyclohexyl)amino)-4-isopropylbenzoic acid (25 mg, 69.16 μmol) in THF (5 mL) were added N³,N³-dimethylpyridazine-3,6-diamine (11.47 mg, 82.99 μmol), BOP (61.18 mg, 138.32 μmol) and TEA (34.99 mg, 345.81 μmol). The mixture was stirred at 50 [symbol] for 16 h. The reaction mixture was cooled to rt and concentrated. The resulting residue was purified by reverse-phase chromatography to give the desired product (18 mg, 54% yield) as a pale-white solid. MS (ESI) m/z=482.6 [M+H]⁺.

### Step 4. Synthesis of trans-4-((2-((6-(dimethylamino)pyridazin-3-yl)carbamoyl)-5-isopropylphenyl)amino)cyclohexane-1-carboxylic acid

To a solution of tert-butyl trans-4-((2-((6-(dimethylamino)pyridazin-3-yl)carbamoyl)-5-isopropylphenyl)amino)cyclohexane-1-carboxylate (18 mg, 37.37 mol) in dichloromethane (2 mL) was added TFA (1 mL). The mixture was stirred at rt for 3 h. The reaction mixture was concentrated to give the crude product (10 mg, 63% yield) as a colorless oil, which was used directly in the next step without further purification. MS(ESI) m/z=426.5 [M+H]⁺.

### Step 5. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-4-isopropylbenzamide

To a mixture of trans-4-((2-((6-(dimethylamino)pyridazin-3-yl)carbamoyl)-5-isopropylphenyl)amino)cyclohexane-1-carboxylic acid (10 mg, 23.50 μmol), EDCI (6.76 mg, 35.25 μmol) and HOAt (4.80 mg, 35.25 μmol) in DMSO (1 mL) were added DIPEA (15.19 mg, 117.50 μmol) and 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (12.14 mg, 23.50 mol). The mixture was stirred at rt for 16 h, before it was purified by prep-HPLC to provide the desired product (1 mg, 5% yield) as a yellow solid MS (ESI) m/z=925.0 [M+H]⁺.

### Example 585. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(5,6-dihydro-4H-pyrrolo[1,2-b]pyrazol-2-yl)-6-(isopropylamino)nicotinamide (CPD-529)

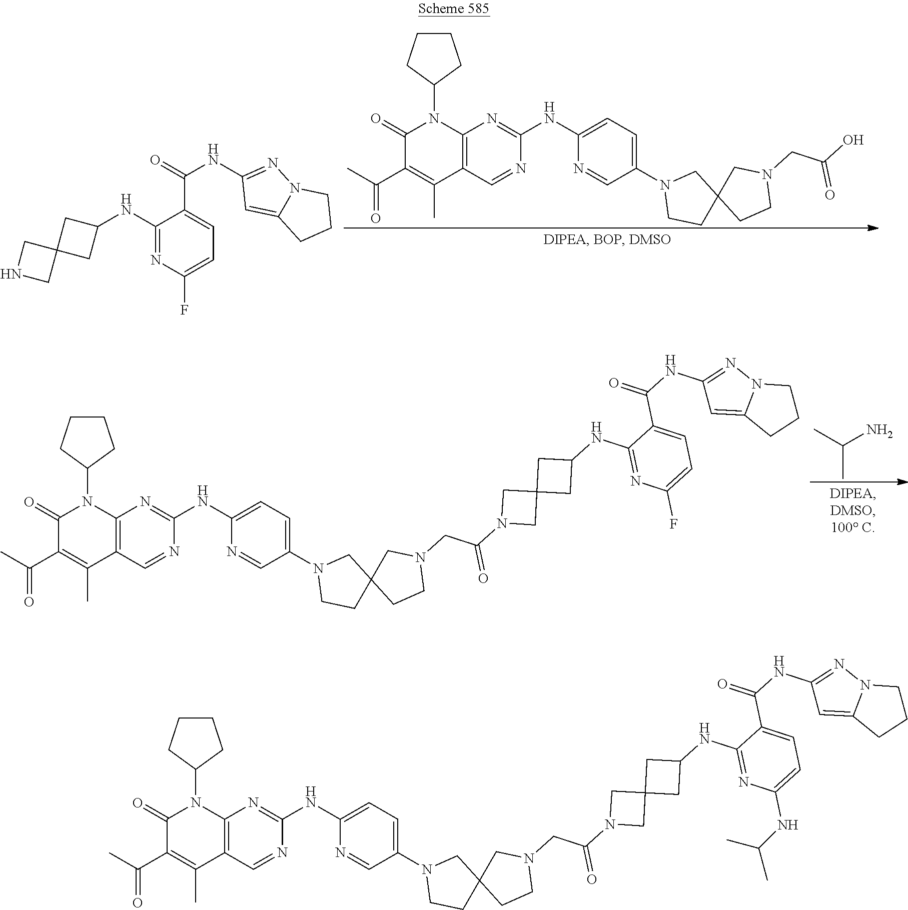

CPD-529 was synthesized following the standard procedures for preparing CPD-477 (10.0 mg, 33% yield over 2 steps) as a yellow solid. MS (ESI) m/z=923.9 [M+H]⁺.

### Example 586. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(5,6-dihydro-4H-pyrrolo[1,2-b]pyrazol-2-yl)-6-(isopropylamino)nicotinamide (CPD-530)

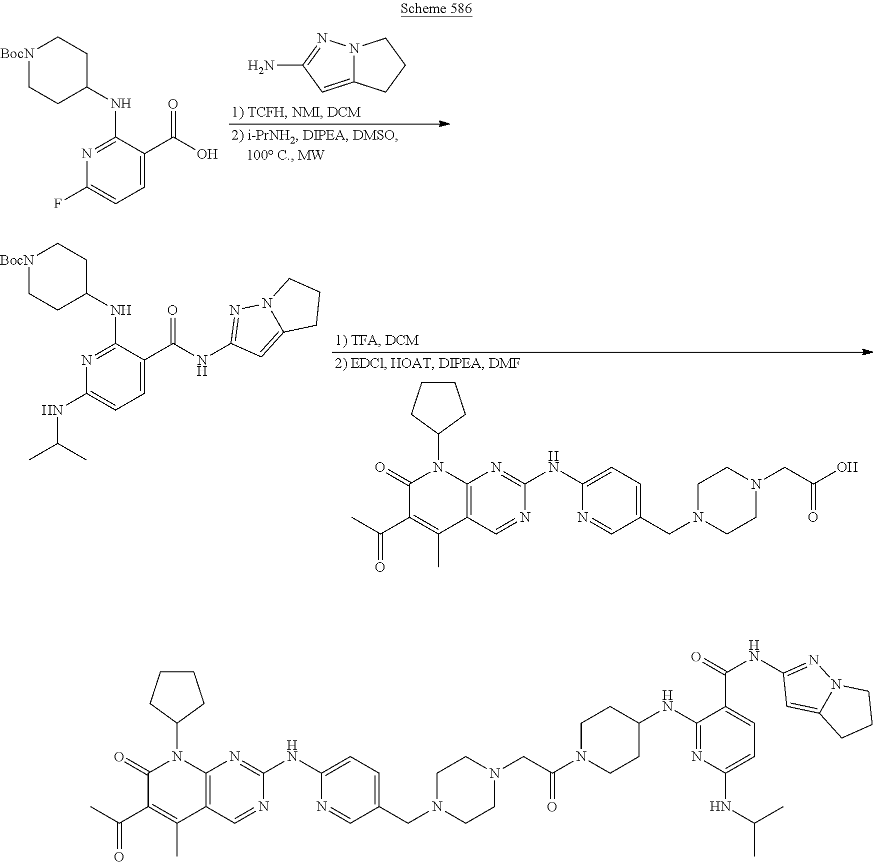

CPD-530 was synthesized following the standard procedures for preparing CPD-144 (8.0 mg, 8% yield over 4 steps) as a yellow solid. MS (ESI) m/z=885.7 [M+H]⁺.

### Example 587. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6-methoxypyridin-2-yl)-6-((methyl-d₃)amino)nicotinamide (CPD-531)

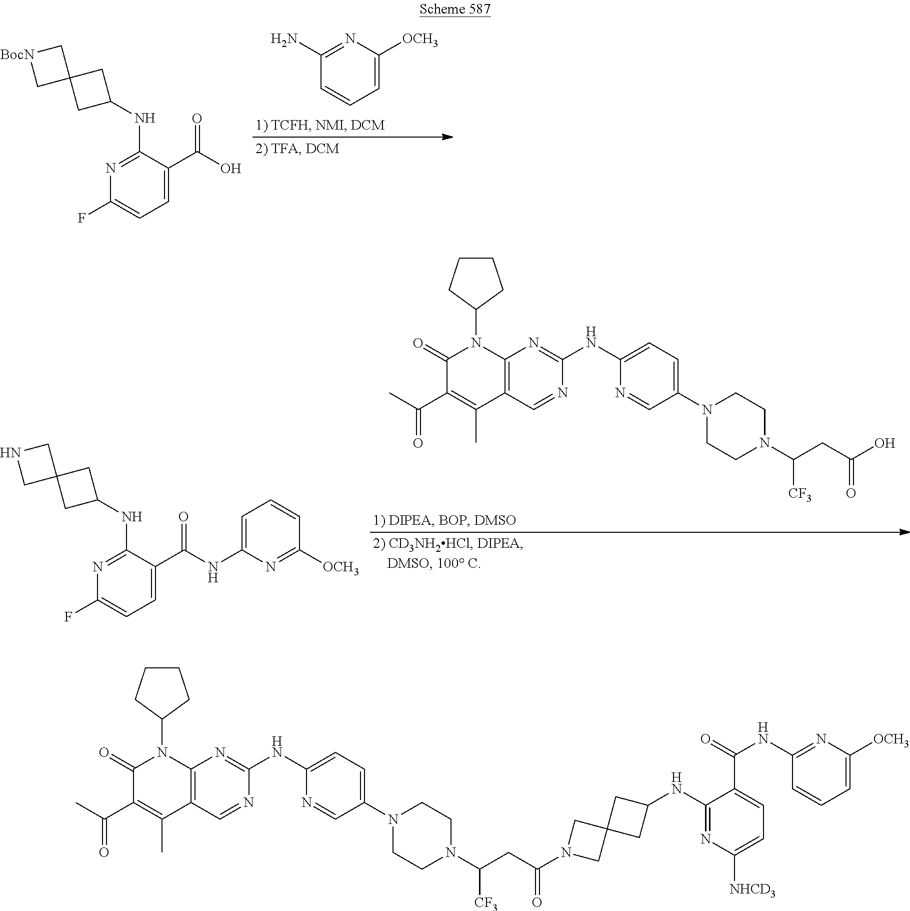

CPD-531 was synthesized following the standard procedures for preparing CPD-477 (9.0 mg, 14% yield over 4 steps) as a yellow solid. MS (ESI) m/z=941.7 [M+H]⁺.

### Example 588. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-6-(isopropylamino)-N-(6-methoxypyridin-2-yl)nicotinamide (CPD-532)

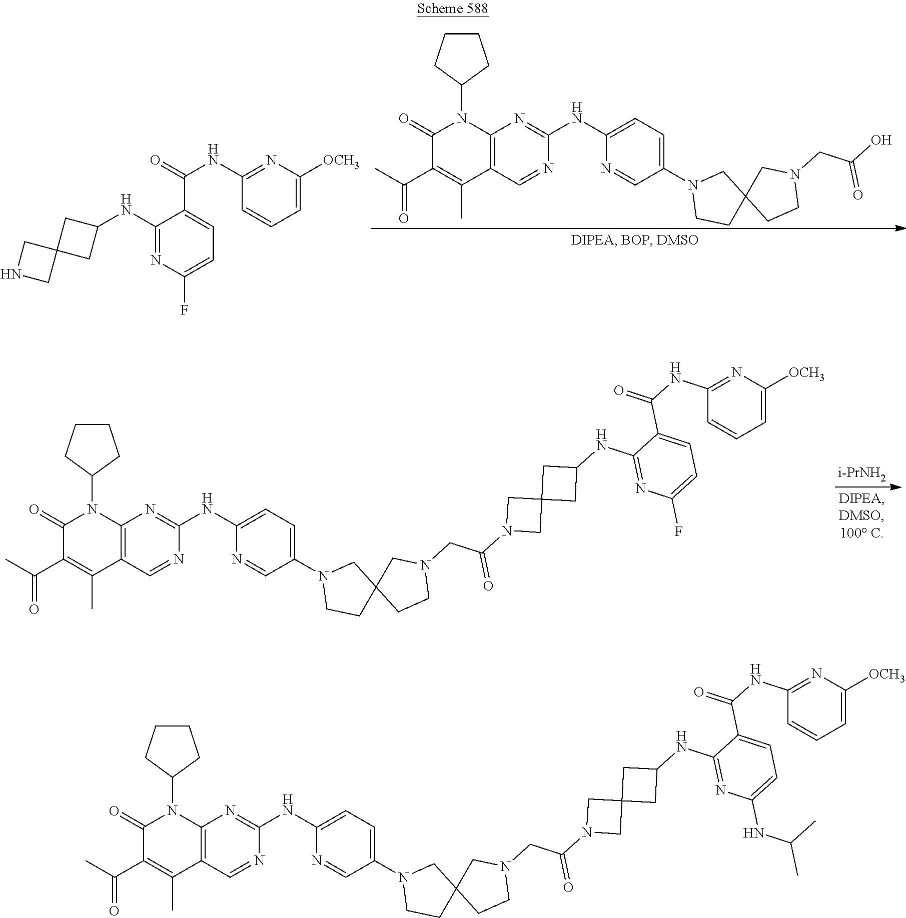 [SMILES: COc1cccc(NC(=O)c2ccc(F)nc2NC2CC3(CNC3)C2)n1 ; CC(=O)c1c(C)c2cnc(Nc3ccc(N4CCC5(CCN(CC(=O)O)C5)C4)cn3)nc2n(C2CCCC2)c1=O ; COc1cccc(NC(=O)c2ccc(F)nc2NC2CC3(C2)CN(C(=O)CN2CCC4(CCN(c5ccc(Nc6ncc7c(C)c(C(C)=O)c(=O)n(C8CCCC8)c7n6)nc5)C4)C2)C3)n1 ; COc1cccc(NC(=O)c2ccc(NC(C)C)nc2NC2CC3(C2)CN(C(=O)CN2CCC4(CCN(c5ccc(Nc6ncc7c(C)c(C(C)=O)c(=O)n(C8CCCC8)c7n6)nc5)C4)C2)C3)n1]

CPD-532 was synthesized following the standard procedures for preparing CPD-477 (10.0 mg, 29% yield over 2 steps) as a yellow solid. MS (ESI) m/z=924.9 [M+H]⁺.

### Example 589. 2-((1-(2-(4-((6-((6-acetyl-8-cyclopentyl-5-methyl-7-oxo-7,8-dihydropyrido[2,3-di pyrimidin-2-yl)amino)pyridin-3-yl)methyl)piperazin-1-yl)acetyl)piperidin-4-yl)amino)-6-(isopropylamino)-N-(6-methoxypyridin-2-yl)nicotinamide (CPD-533)

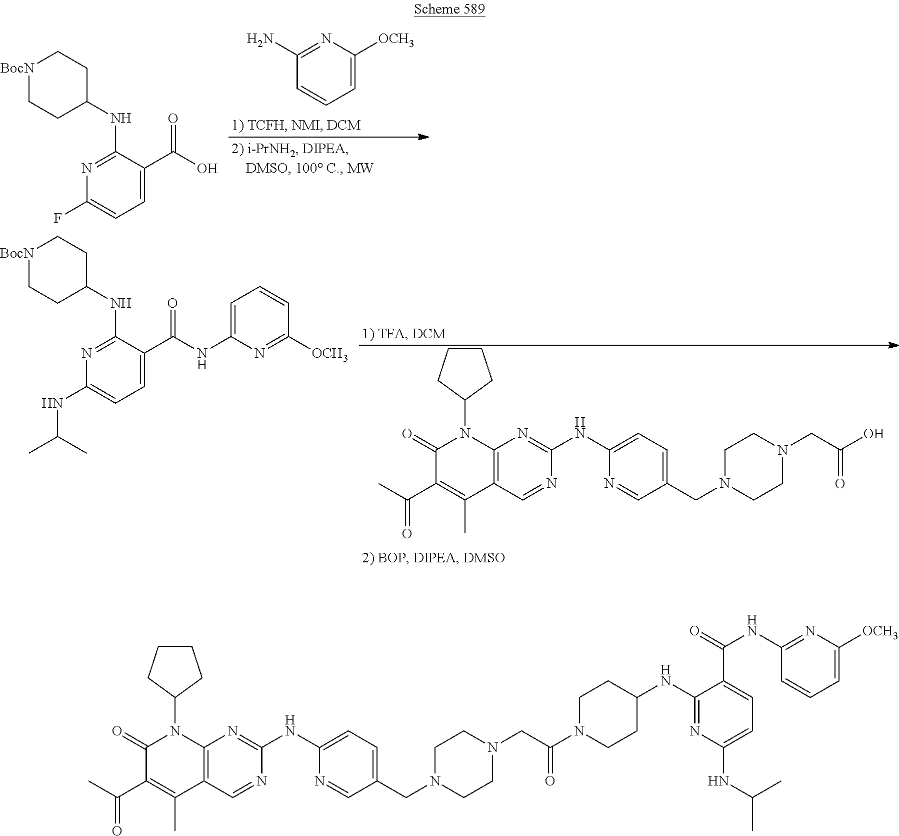

CPD-533 was synthesized following the standard procedures for preparing CPD-144 (5.0 mg, 4% yield over 4 steps) as a yellow solid. MS (ESI) m/z=886.7 [M+H]⁺.

### Example 590. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-(isopropylamino)nicotinamide (CPD-534)

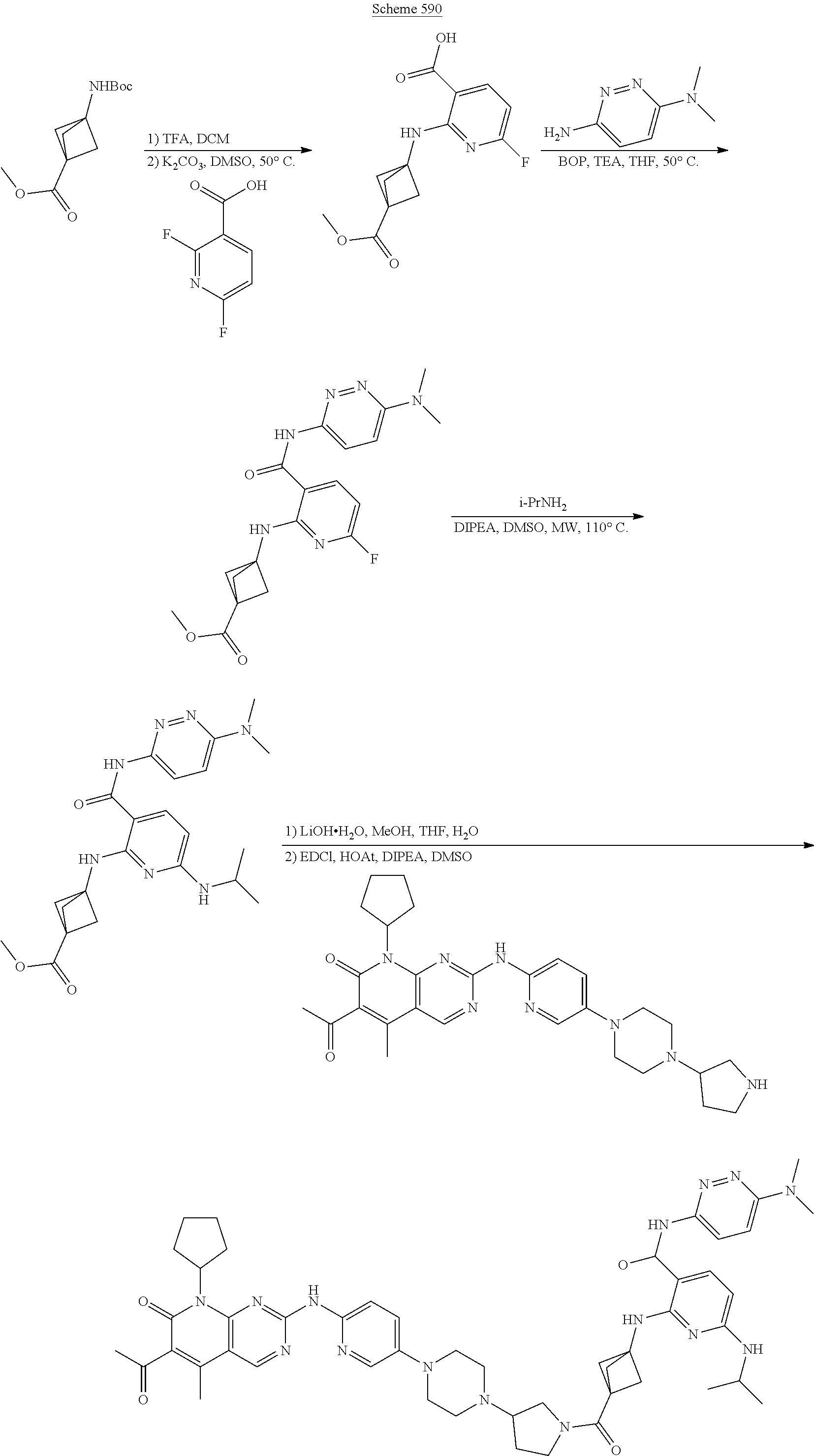

CPD-534 was synthesized following the standard procedures for preparing CPD-82 (7.5 mg, 4% yield over 6 steps) as a yellow solid. MS (ESI) m/z=924.7 [M+H]⁺.

### Example 591. 2-((4-(3-(4-(6-((8-cyclopentyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dim ethylamino)pyridazin-3-yl)-6-(isopropylamino)nicotinamide (CPD-535)

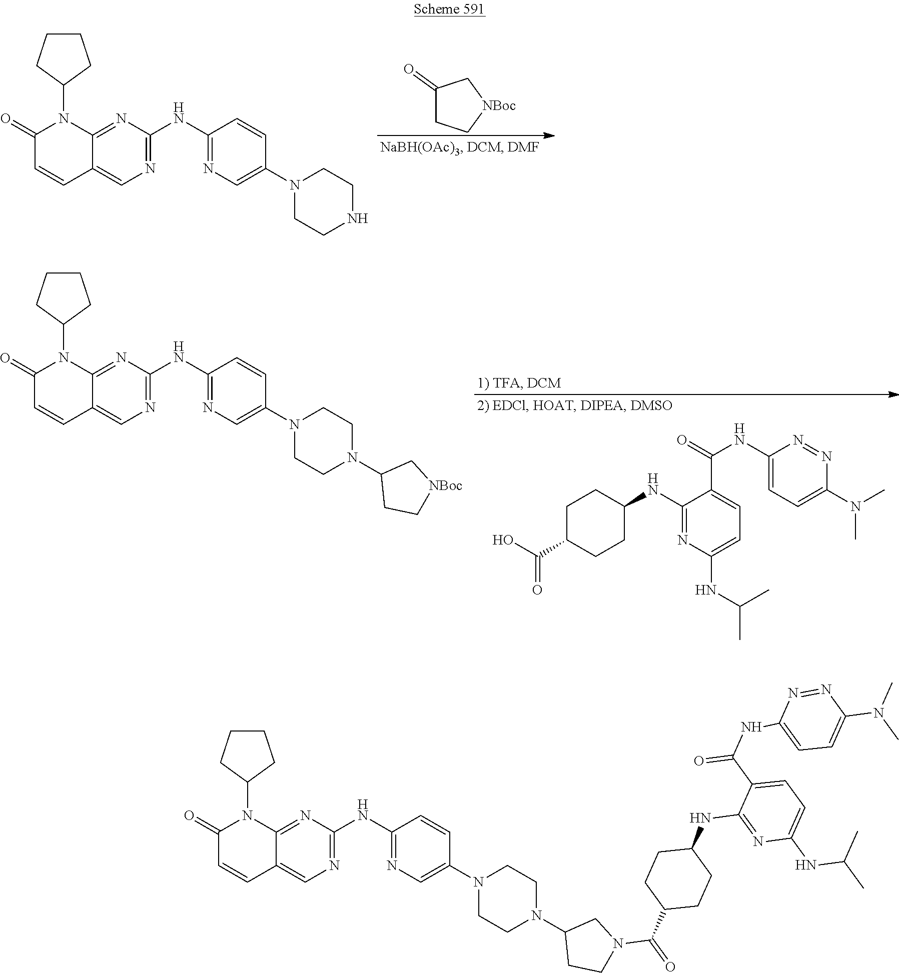

CPD-535 was synthesized following the standard procedures for preparing CPD-510 (8.4 mg, 13% yield over 3 steps) as a yellow solid. MS (ESI) m/z=884.7 [M+H]⁺.

### Example 592. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-methoxycyclohexyl)amino)-N-(6-(dimethylamino)pyridazin-3-yl)-6-(isopropylamino)nicotinamide (CPD-536)

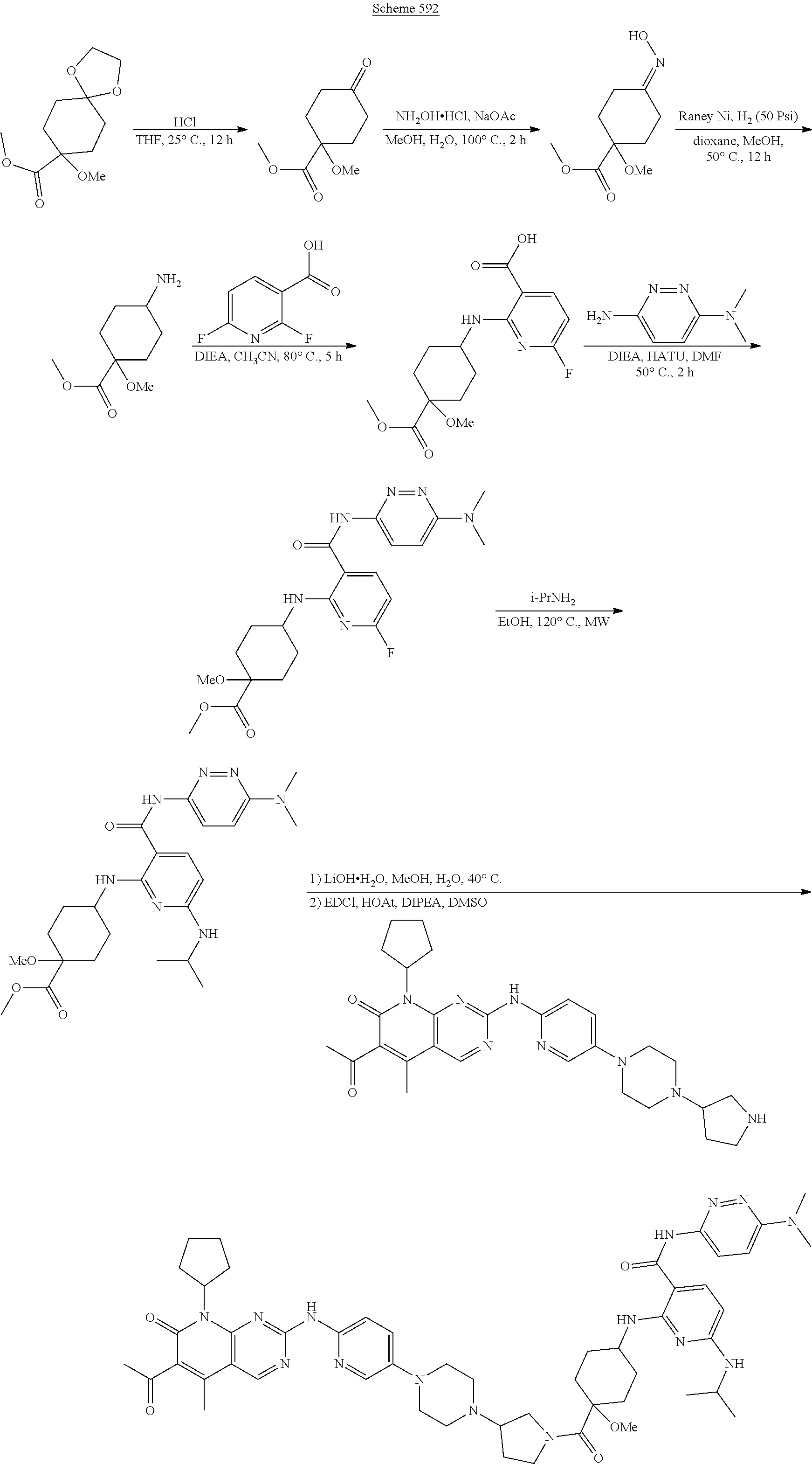

CPD-536 was synthesized following the standard procedures for preparing CPD-516 and CPD-82 (2.5 mg, 1% yield over 8 steps) as a yellow solid. MS (ESI) m/z=971.1 [M+H]⁺.

### Example 593. 2-((3-(2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidin-1-yl)-2-oxoethyl)bicyclo[1.1.1]pentan-1-yl)amino)-N-(6-(dimethylamino)pyridazin-3-yl)-6-(isopropylamino)nicotinamide (CPD-537)

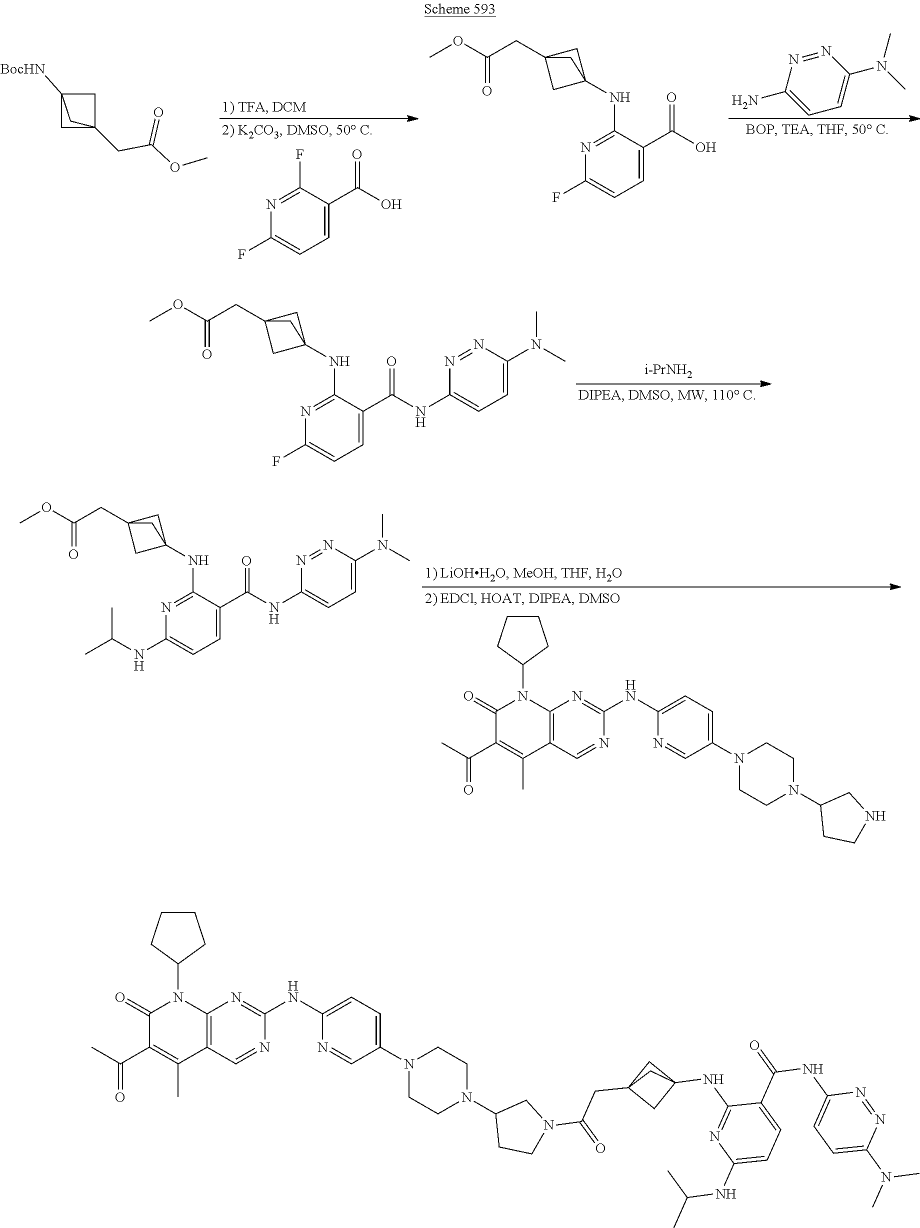

CPD-537 was synthesized following the standard procedures for preparing CPD-82 (5.9 mg, 5% yield over 6 steps) as a yellow solid. MS (ESI) m/z=938.8 [M+H]⁺.

### Example 594. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)-2-azaspiro[3.3]heptan-6-yl)amnion)-N-(6-(dimethylamino)pyridazin-3-yl)-6-(isopropylamino)nicotinamide (CPD-538)

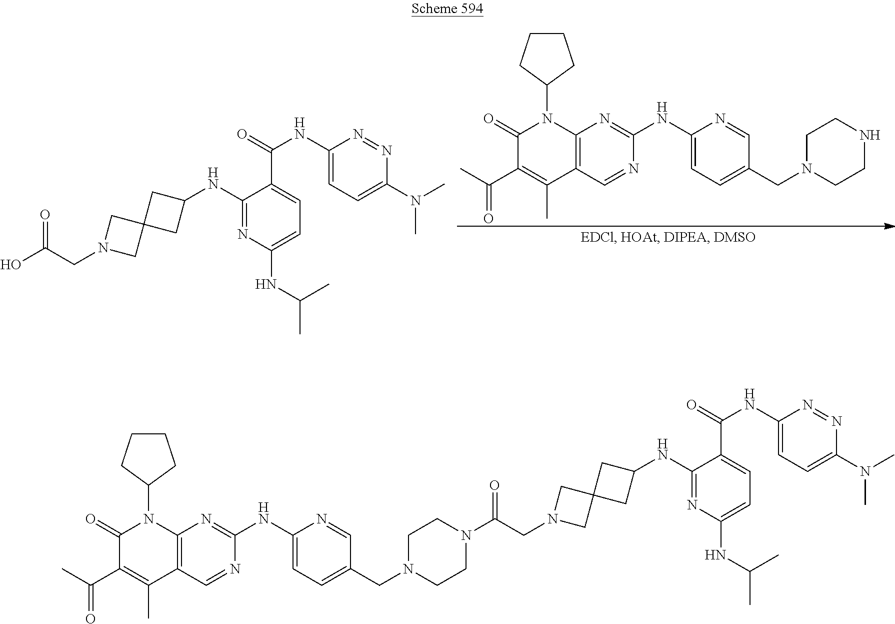

CPD-538 was synthesized following the standard procedure for preparing CPD-143 (1.2 mg, 10% yield) as a yellow solid. MS (ESI) m/z=912.9 [M+H]⁺.

### Example 595. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-6-azaspiro[3.4]octan-2-yl)amino)-N-(6-(dimethylamino)pyridazin-3-yl)-6-(isopropylamino)nicotinamide (CPD-539)

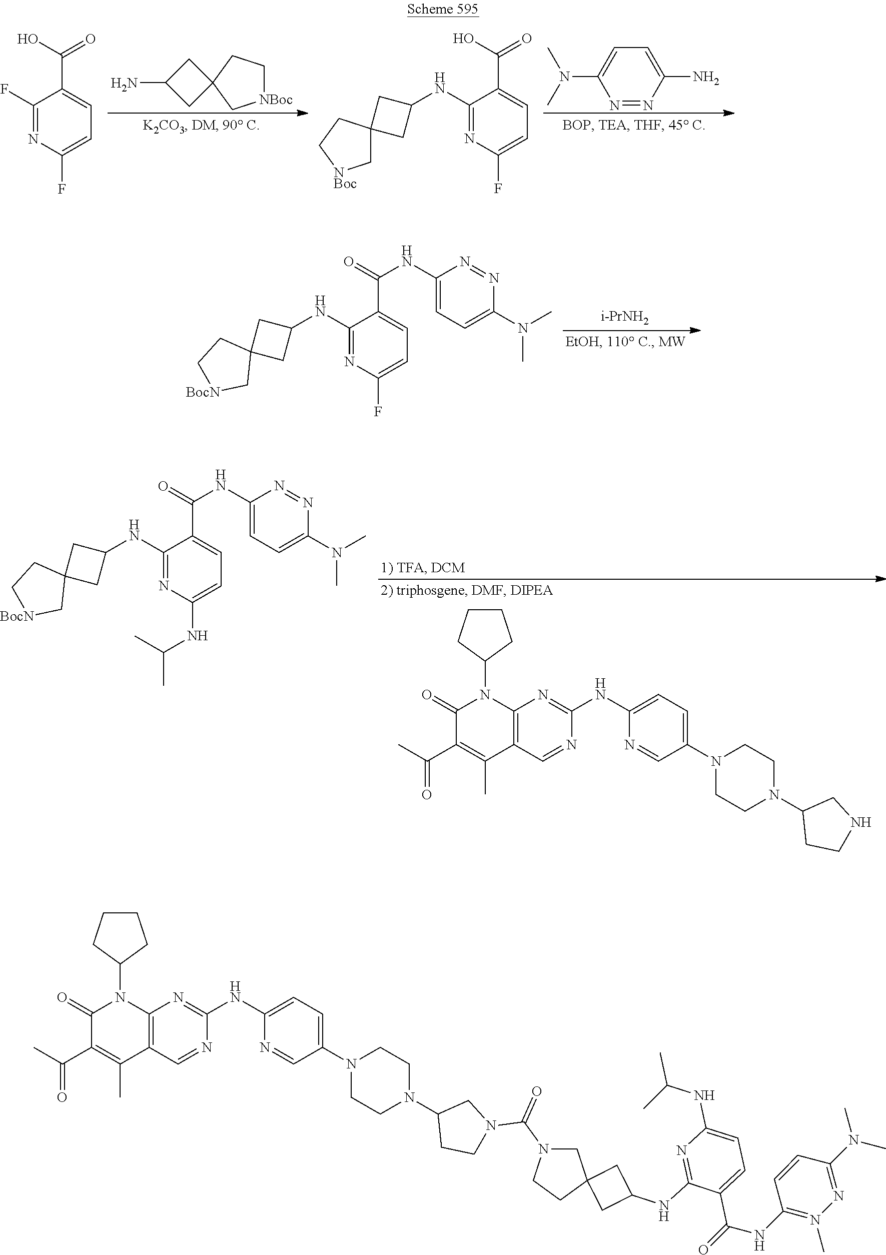

CPD-539 was synthesized following the standard procedures for preparing CPD-145 and CPD-469 (0.9 mg, 10 yield over 5 steps) as a yellow solid. MS (ESI) m/z=968.1 [M+H]⁺.

### Example 596. trans-6-acetyl-8-cyclopentyl-2-((5-(4-(1-(4-((3-(5-(dimethylamino)-1H-benzo[d]imidazol-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one (CPD-540)

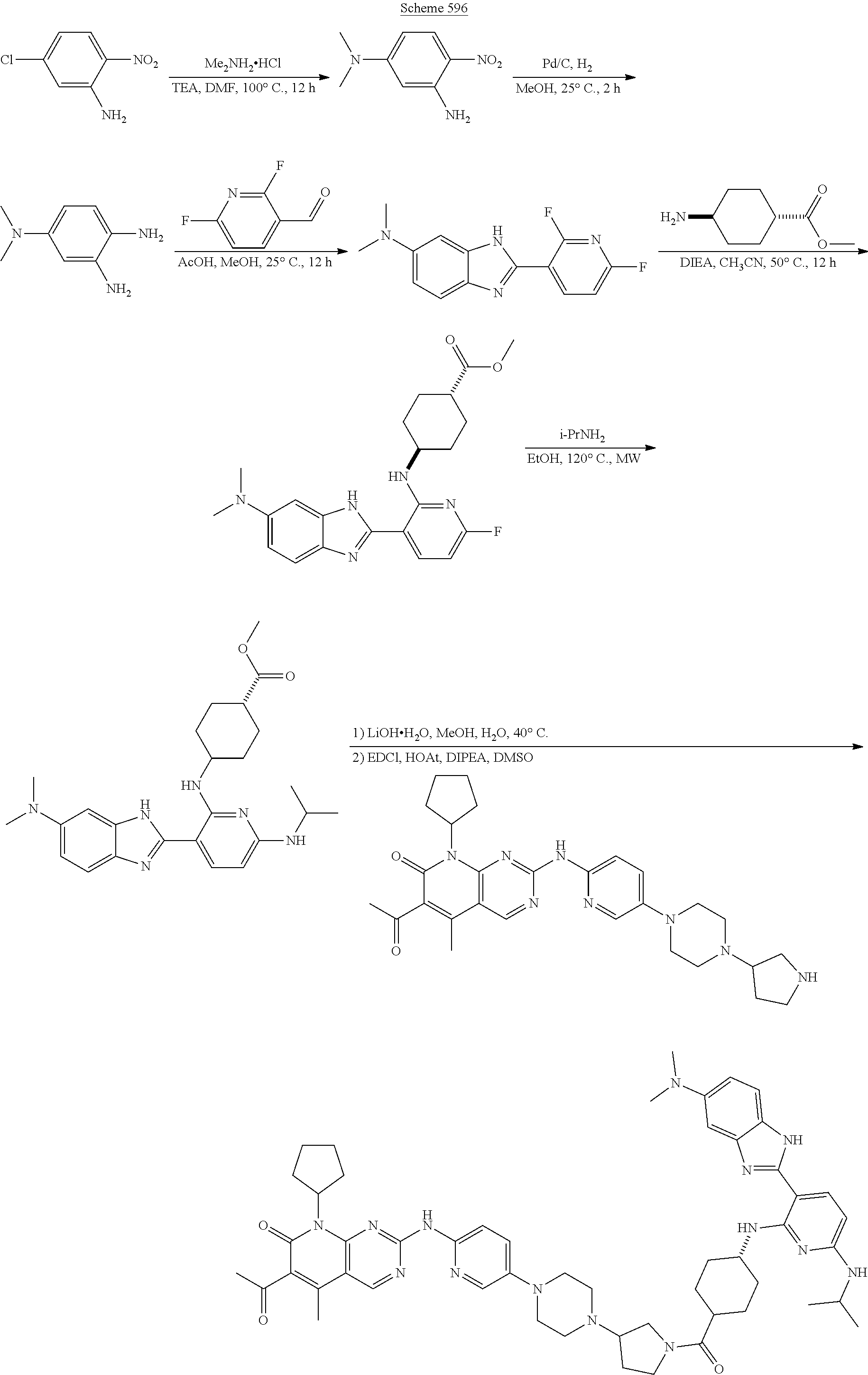

### Step 1. Synthesis of N,N-dimethyl-4-nitrobenzene-1,3-diamine

To a solution of 5-chloro-2-nitro-aniline (5.00 g, 28.9 mmol) in DMF (100 mL) were added TEA (20.5 g, 202 mmol) and dimethylamine (11.8 g, 144 mmol, HCl). The mixture was stirred at 100° C. for 12 h. The resulting mixture was poured into H₂O (500 mL) and extracted with ethyl acetate (100 mL×3). The combined organic layers were washed with brine (300 mL), dried over Na₂SO₄, filtered and concentrated.

The residue was purified by column chromatography (SiO₂, petroleum ether/ethyl acetate=5:1 to 3:1) to provide the desired product (2.40 g, 45% yield) as a yellow solid. ¹HNMR (400 MHz, CDCl₃) δ 8.01 (d, J=9.6 Hz, 1H), 6.17-6.12 (m, 3H), 5.73 (d, J=2.8 Hz, 1H), 3.05 (s, 6H).

### Step 2. Synthesis of N⁴,N⁴-dimethylbenzene-1,2,4-triamine

To a solution of N¹,N¹-dimethyl-4-nitrobenzene-1,3-diamine (1.80 g, 9.93 mmol) in MeOH (18 mL) was [symbol] added 10% Pd/C (360 mg) at 25c. The mixture was stirred at 25 [symbol] for 2 h under H₂ (15 psi). The mixture was filtered and concentrated under vacuum to provide the desired product (1.60 g, crude) as a brown oil. ¹HNMR (400 MHz, CDCl₃) δ 6.65 (d, J 8.4 Hz, 1H), 6.25 (d, J 2.4 Hz, 1H), 6.20 (d, J 7.6 Hz, 1H), 2.83 (s, 6H).

### Step 3. Synthesis of 2-(2,6-difluoropyridin-3-yl)-N,N-dimethyl-1H-benzo[d]imidazol-6-amine

To a solution of 2,6-difluoropyridine-3-carbaldehyde (1.60 g, 11.2 mmol) and N⁴,N⁴-dimethylbenzene-1,2,4-triamine (1.60 g, 10.6 mmol) in MeOH (16 mL) was added AcOH (5.56 g, 92.6 mmol). The mixture was stirred at 25° C. for 12 h. The resulting mixture was concentrated under vacuum to remove most of AcOH and MeOH. The residue was poured into H₂O (1000 mL) and extracted with ethyl acetate (300 mL×3). The combined organic phase was washed with brine (500 mL×3), dried with anhydrous Na₂SO₄, filtered, and concentrated under vacuum to provide the desired product (2.90 g, 99% yield) as a black solid. MS (ESI) m/z=275.1 [M+H]⁺.

The remaining steps were performed according to the procedures for preparing CPD-82 to provide the desired product (4.0 mg, 1% yield over 4 steps) as a yellow solid. MS (ESI) m/z=935.9 [M+H]⁺.

### Example 597. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-2-oxoethyl)piperidin-4-yl)amino)-N-(6-(dimethylamino)pyridazin-3-yl)-6-(isopropylamino)nicotinamide (CPD-541)

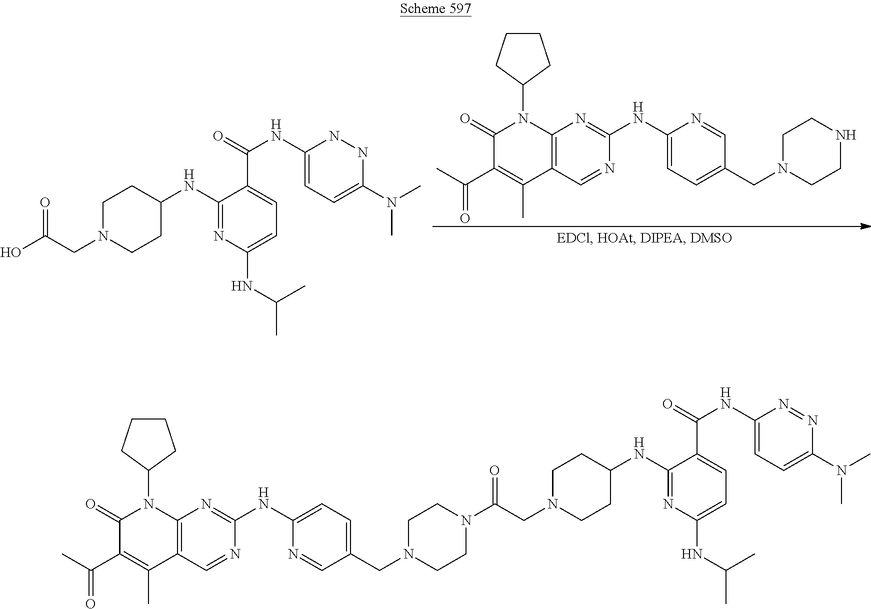

CPD-541 was synthesized following the standard procedure for preparing CPD-143 (3.7 mg, 20% yield) as a yellow solid. MS (ESI) m/z=900.7 [M+H]⁺.

### Example 598. (S)-2-((3-((1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-2-carboxamido)methyl)cyclobutyl)amino)-N-(6-(dimethylamino)pyridazin-3-yl)-6-(isopropylamino)nicotinamide (CPD-542)

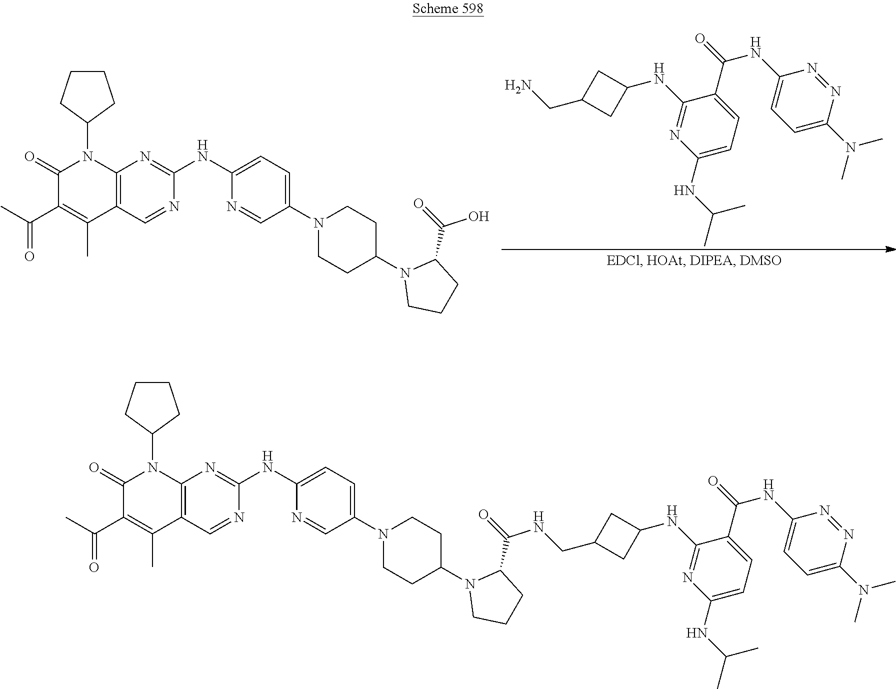 [SMILES: CC(=O)c1c(C)c2cnc(Nc3ccc(N4CCC(N5CCC[C@H]5C(=O)O)CC4)cn3)nc2n(C2CCCC2)c1=O ; CC(C)Nc1ccc(C(=O)Nc2ccc(N(C)C)nn2)c(NC2CC(CN)C2)n1 ; CC(=O)c1c(C)c2cnc(Nc3ccc(N4CCC(N5CCC[C@H]5C(=O)NCC5CC(Nc6nc(NC(C)C)ccc6C(=O)Nc6ccc(N(C)C)nn6)C5)CC4)cn3)nc2n(C2CCCC2)c1=O]

CPD-542 was synthesized following the standard procedure for preparing CPD-143 (8.4 mg, 44% yield) as a yellow solid. MS (ESI) m/z=940.7 [M+H]⁺.

### Example 599. (S)-2-((6-(1-(1-(6-((6-acetyl-8-cyclopentyl-5-methyl-7-oxo-7,8-dihydropyrido[2,3-d]pyrimidin-2-yl)amino)pyridin-3-yl)piperidin-4-yl)pyrrolidine-2-carboxamido)spiro[3.3]heptan-2-yl)amino)-N-(6-(dimethylamino)pyridazin-3-yl)-6-(isopropylamino)nicotinamide (CPD-543)

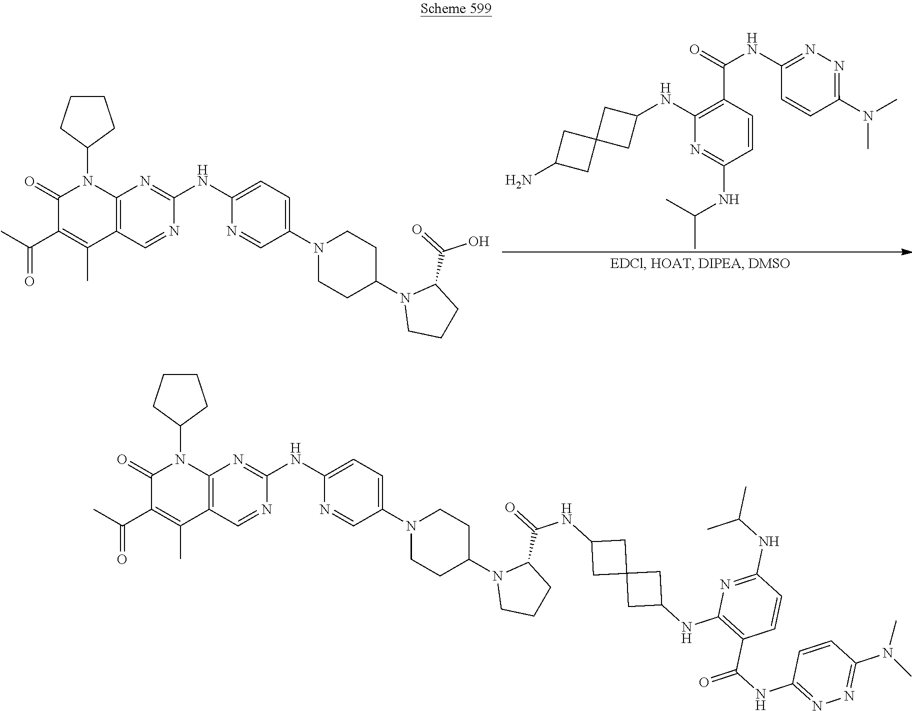

CPD-543 was synthesized following the standard procedure for preparing CPD-143 (8.4 mg, 43% yield) as a yellow solid. MS (ESI) m/z=966.8 [M+H]⁺.

### Example 600. 2-((2-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)-L-prolyl)-2-azaspiro[3.3]heptan-6-yl)amino)-N-(6-(dimethylamino)pyridazin-3-yl)-6-(isopropylamino)nicotinamide (CPD-544)

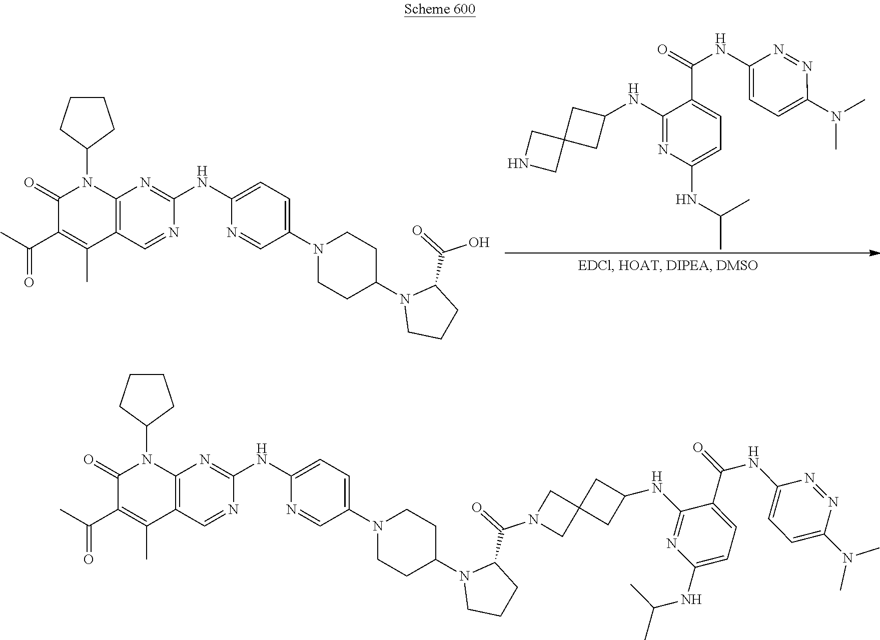

CPD-544 was synthesized following the standard procedure for preparing CPD-143 (6.5 mg, 34% yield) as a yellow solid. MS (ESI) m/z=952.8 [M+H]⁺.

### Example 601. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[3.4]octan-6-yl)amino)-N-(6-(dimethylamino)pyridazin-3-yl)-6-(isopropylamino)nicotinamide (CPD-545)

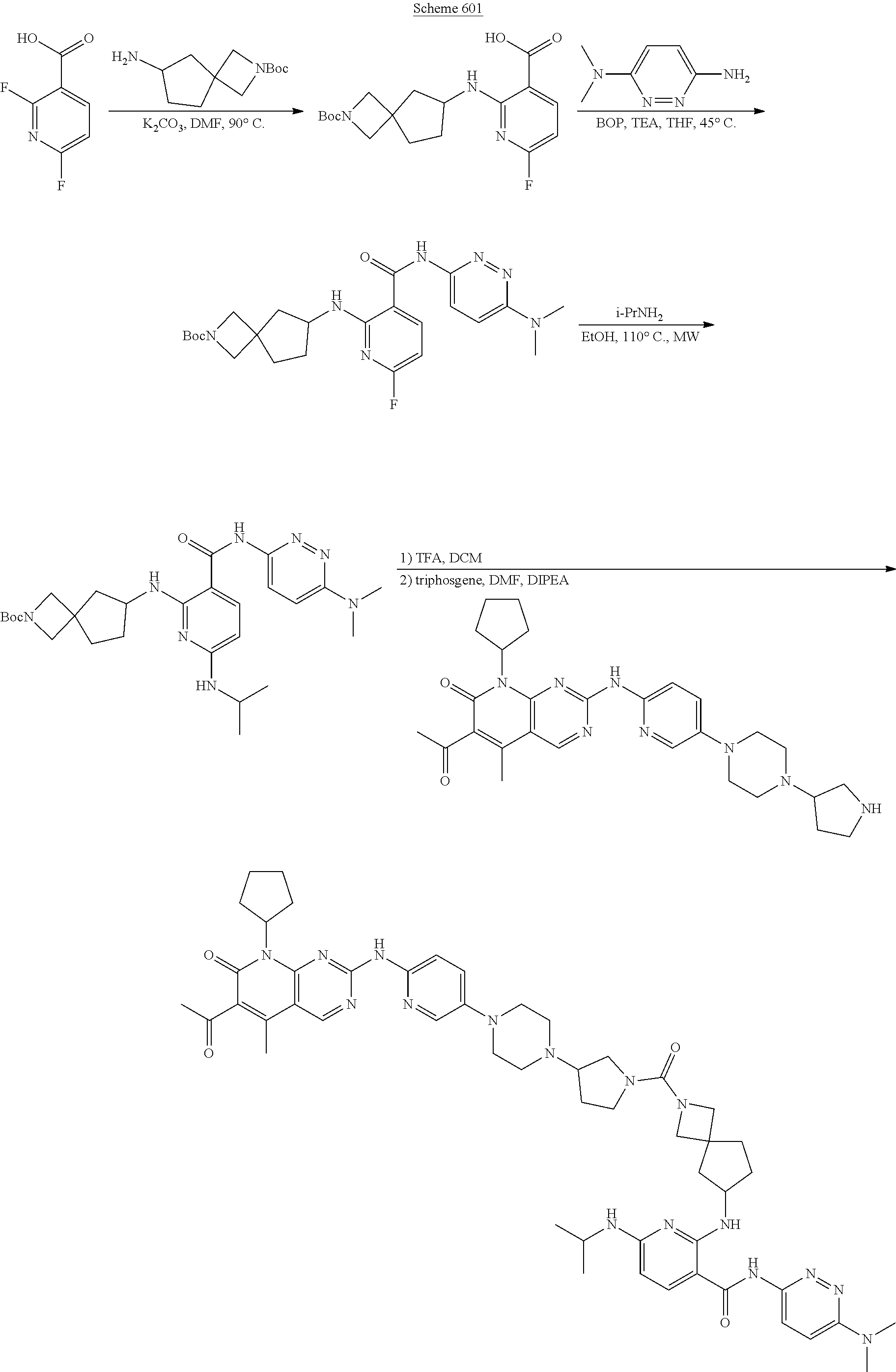

CPD-545 was synthesized following the standard procedures for preparing CPD-145 and CPD-469 (1.3 mg, 8% yield over 5 steps) as a light-yellow solid. MS (ESI) m/z=968.2 [M+H]⁺.

### Example 602. trans-6-acetyl-8-cyclopentyl-2-((5-(4-(1-(4-((3-(5-(dimethylamino)-3H-imidazo[4,5-b]pyridin-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one (CPD-546)

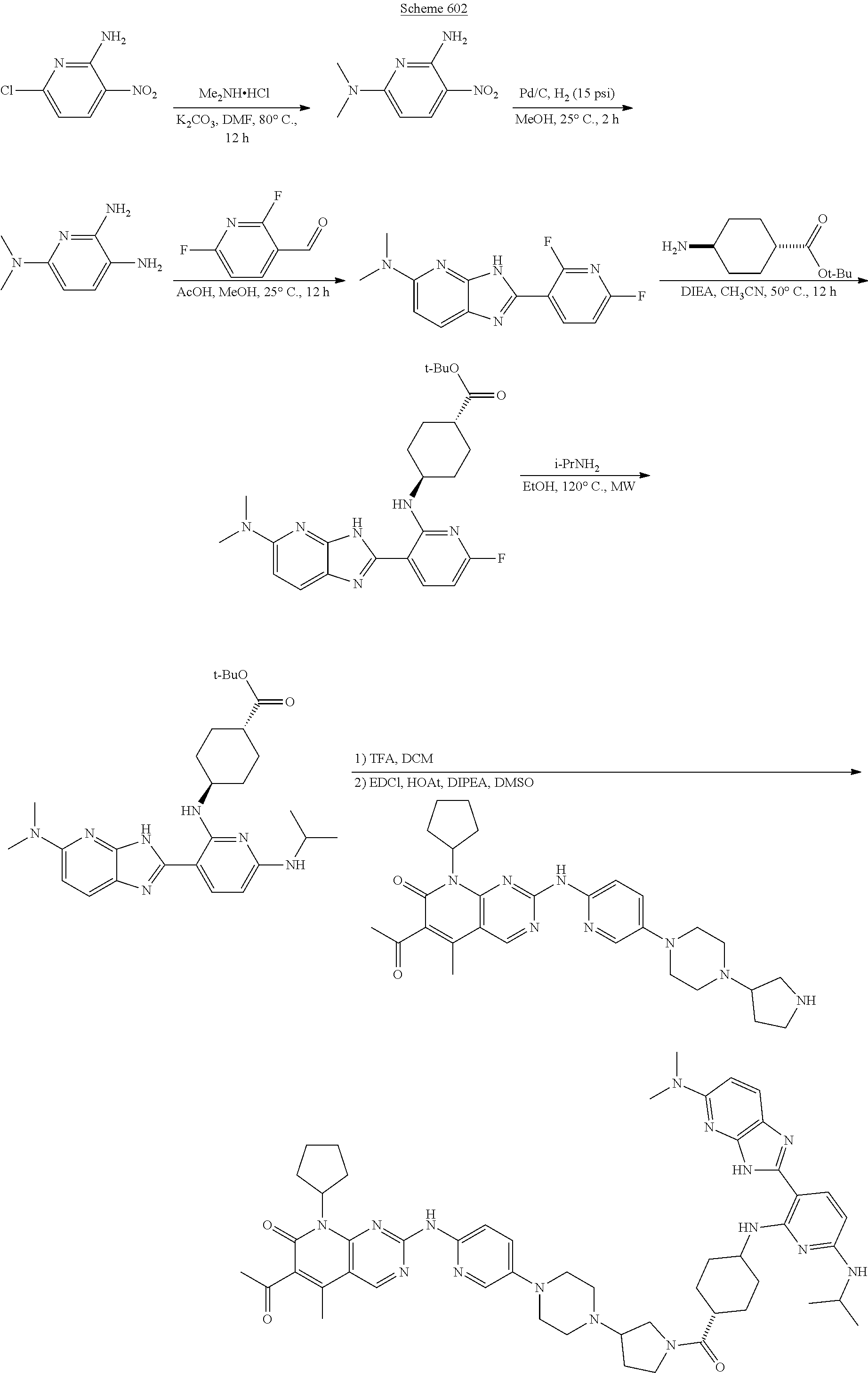

CPD-546 was synthesized following the standard procedures for preparing CPD-540 and CPD-144 (2.0 mug, 1% yield over 7 steps) as a light-yellow solid. MS (ESI) m/z=937.0 [M+H]⁺.

### Example 603. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(1,5-dimethyl-1H-pyrazol-3-yl)-6-(isopropylamino)nicotinamide (CPD-547)

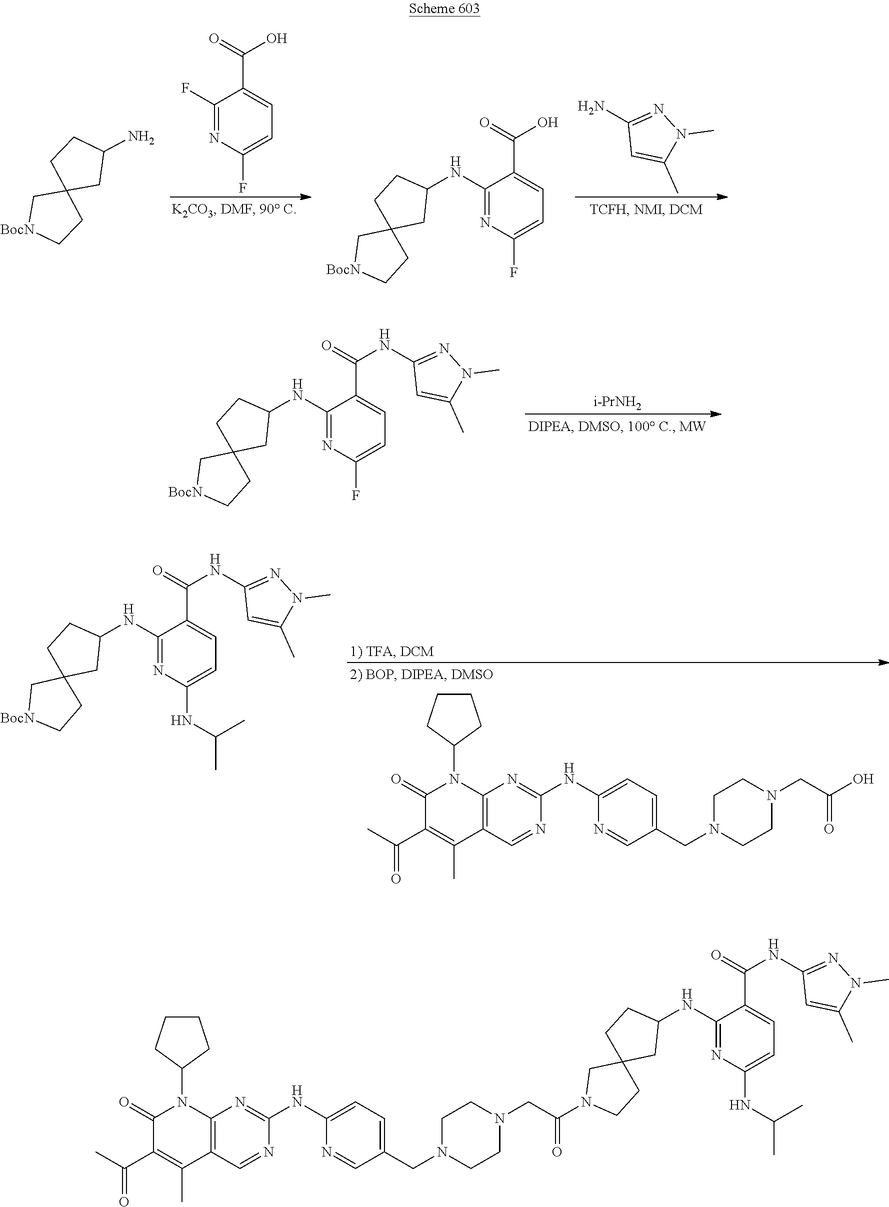

CPD-547 was synthesized following the standard procedures for preparing CPD-144 (6.0 mg, 8% yield over 5 steps) as a yellow solid. MS (ESI) m/z=913.7 [M+H]⁺.

### Example 604. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(2-cyclopropyl-1-methyl-1H-imidazol-4-yl)-6-(isopropylamino)nicotinamide (CPD-548)

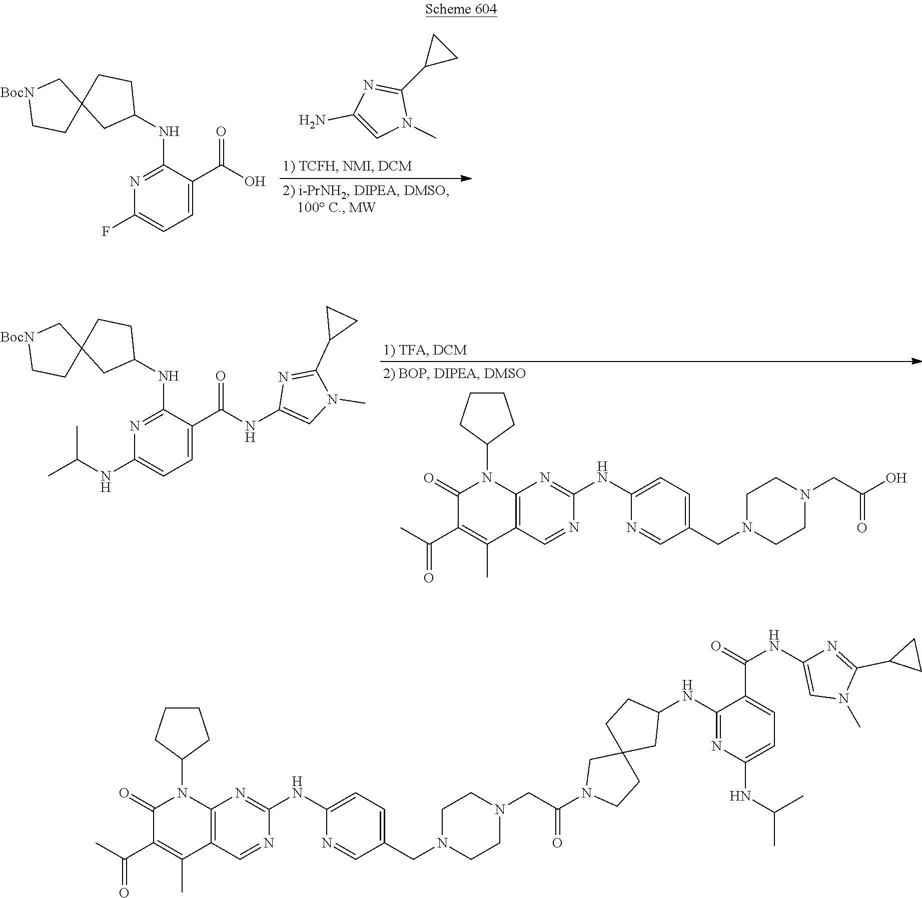

CPD-548 was synthesized following the standard procedures for preparing CPD-144 (6.0 mg, 8% yield over 4 steps) as a yellow solid. MS (ESI) m/z=939.8 [M+H]⁺.

### Example 605. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(5,6-dihydro-4H-pyrrolo[1,2-b]pyrazol-2-yl)-6-(isopropylamino)nicotinamide (CPD-549)

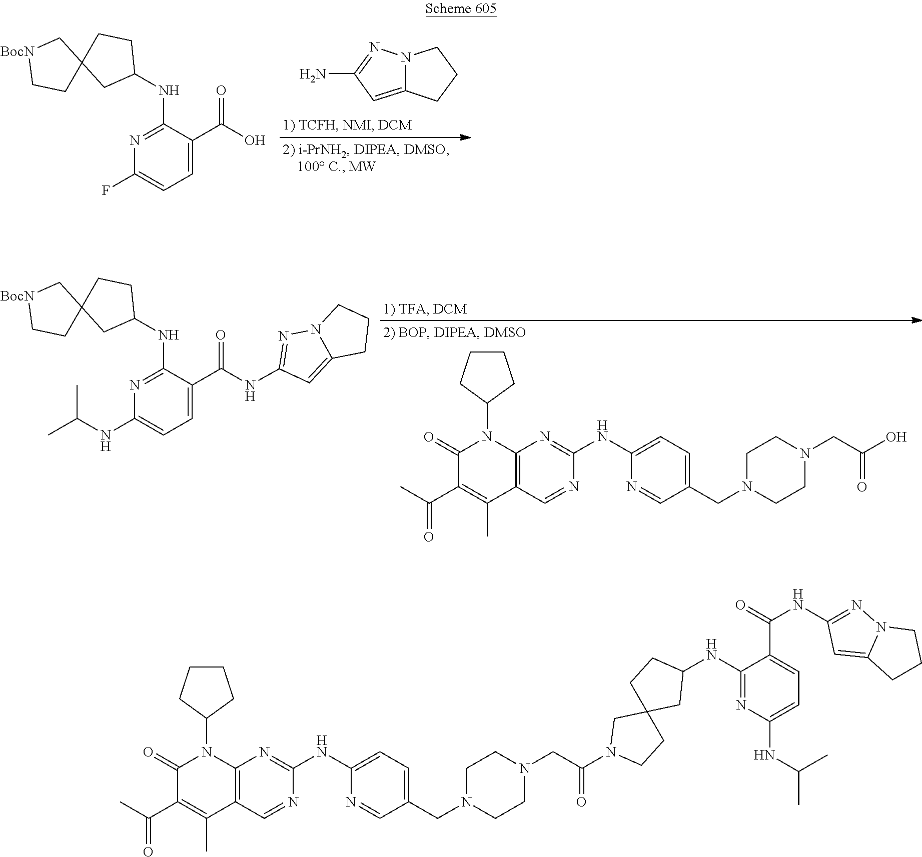 [SMILES: CC(C)(C)OC(=O)N1CCC2(CCC(Nc3nc(F)ccc3C(=O)O)C2)C1 ; Nc1cc2n(n1)CCC2 ; CC(C)Nc1ccc(C(=O)Nc2cc3n(n2)CCC3)c(NC2CCC3(CCN(C(=O)OC(C)(C)C)C3)C2)n1 ; CC(=O)c1c(C)c2cnc(Nc3ccc(CN4CCN(CC(=O)O)CC4)cn3)nc2n(C2CCCC2)c1=O ; CC(=O)c1c(C)c2cnc(Nc3ccc(CN4CCN(CC(=O)N5CCC6(CCC(Nc7nc(NC(C)C)ccc7C(=O)Nc7cc8n(n7)CCC8)C6)C5)CC4)cn3)nc2n(C2CCCC2)c1=O]

CPD-549 was synthesized following the standard procedures for preparing CPD-144 (7.0 mg, 9% yield over 4 steps) as a yellow solid. MS (ESI) m/z=925.7 [M+H]⁺.

### Example 606. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanoyl)-2-azaspiro[4.4]nonan-7-yl)amino)-N-(6-(dimethylamino)pyridazin-3-yl)-6-(isopropylamino)nicotinamide (CPD-550)

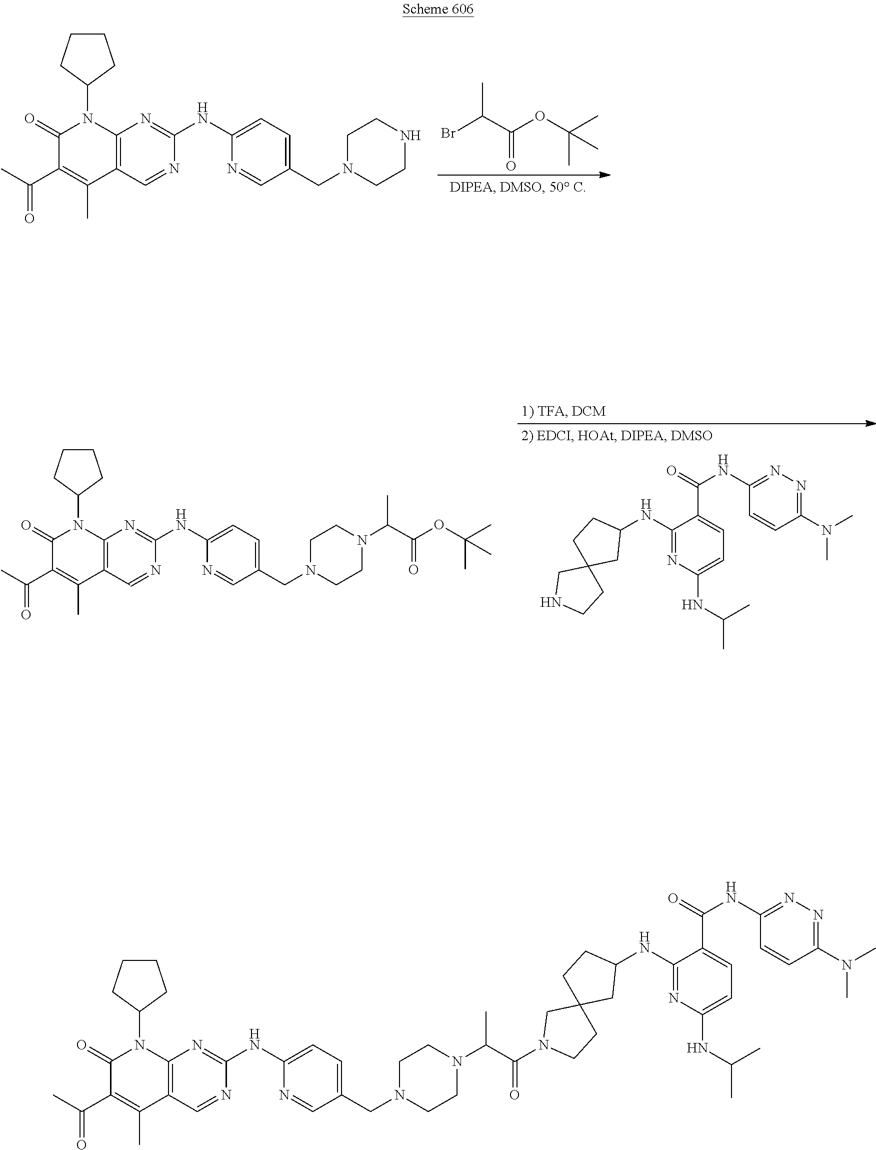

CPD-550 was synthesized following the standard procedures for preparing CPD-418 (3.6 mg, 20% yield over 3 steps) as a yellow solid. MS (ESI) m/z=954.9 [M+H]⁺.

### Example 607. 2-((2-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetyl)-2-azaspiro[4.4]nonan-7-yl)amino)-N-(6-(dimethylamino)pyridazin-3-yl)-6-(isopropylamino)nicotinamide (CPD-551)

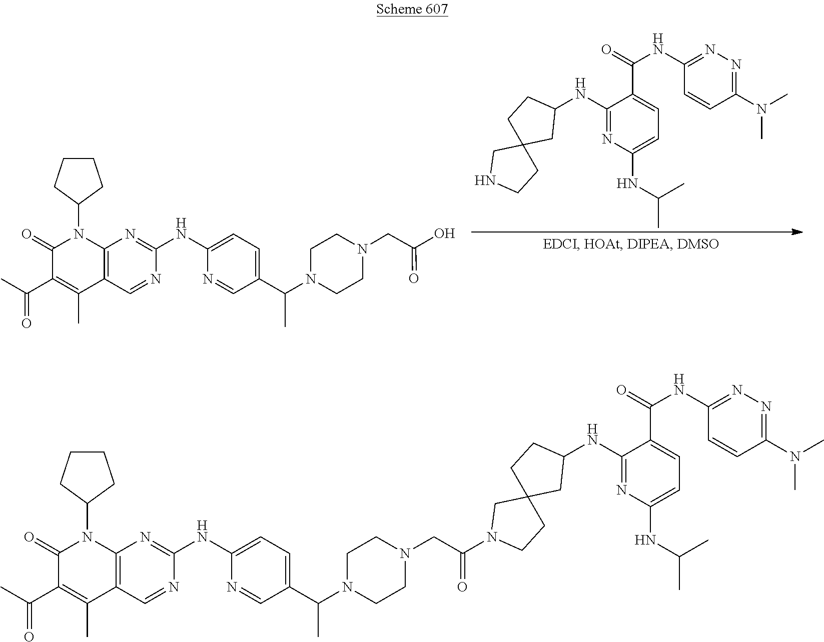 [SMILES: CC(=O)c1c(C)c2cnc(Nc3ccc(C(C)N4CCN(CC(=O)O)CC4)cn3)nc2n(C2CCCC2)c1=O ; CC(C)Nc1ccc(C(=O)Nc2ccc(N(C)C)nn2)c(NC2CCC3(CCNC3)C2)n1 ; CC(=O)c1c(C)c2cnc(Nc3ccc(C(C)N4CCN(CC(=O)N5CCC6(CCC(Nc7nc(NC(C)C)ccc7C(=O)Nc7ccc(N(C)C)nn7)C6)C5)CC4)cn3)nc2n(C2CCCC2)c1=O]

CPD-551 was synthesized following the standard procedure for preparing CPD-143 (2.3 mg, 25% yield) as a yellow solid. MS (ESI) m/z=954.9 [M+H]⁺.

### Example 608. trans-6-acetyl-8-cyclopentyl-2-((5-(4-(1-(trans-4-((3-(6-(dimethylamino)-3H-imidazo[4,5-c]pyridin-2-yl)-6-(isopropylamino)pyridin-2-yl)amino)cyclohexane-1-carbonyl)pyrrolidin-3-yl)piperazin-1-yl)pyridin-2-yl)amino)-5-methylpyrido[2,3-d]pyrimidin-7(8H)-one (CPD-552)

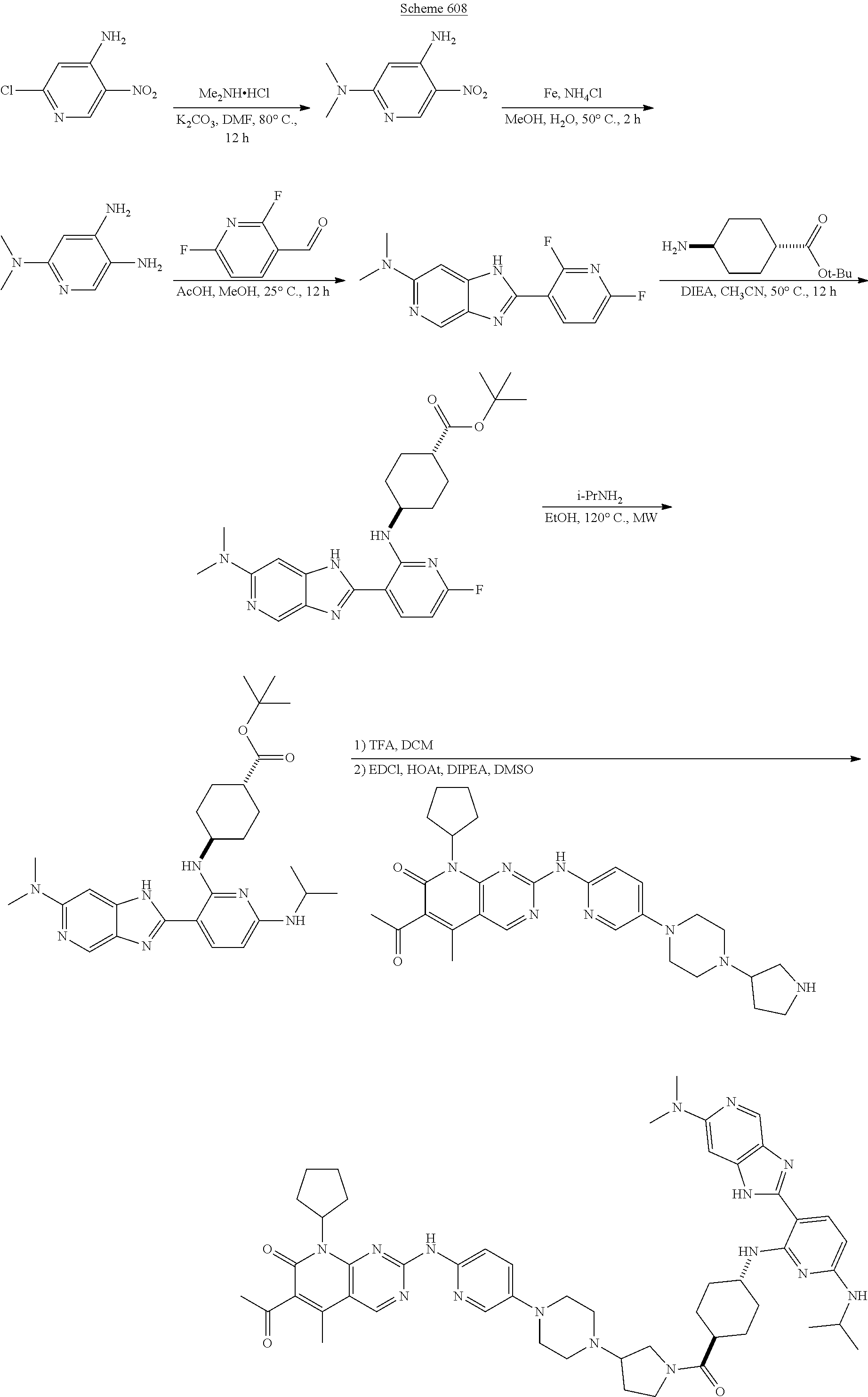

CPD-552 was synthesized following the standard procedures for preparing CPD-540 and CPD-144 (2.9 mg, 1% yield over 7 steps) as a yellow solid. MS (ESI) m/z=937.0 [M+H]⁺.

### Example 609. N-(6-(dimethylamino)pyridazin-3-yl)-2-((2-(2-(4-((6-((4-(2-ethyl-1-oxo-1,2,3,4-tetrahydroisoquinolin-6-yl)-5-fluoropyrimidin-2-yl)amino)pyridin-3-yl)methyl)piperazin-1-yl)acetyl)-2-azaspiro[3.3]heptan-6-yl)amino)-6-(isopropylamino)nicotinamide (CPD-553)

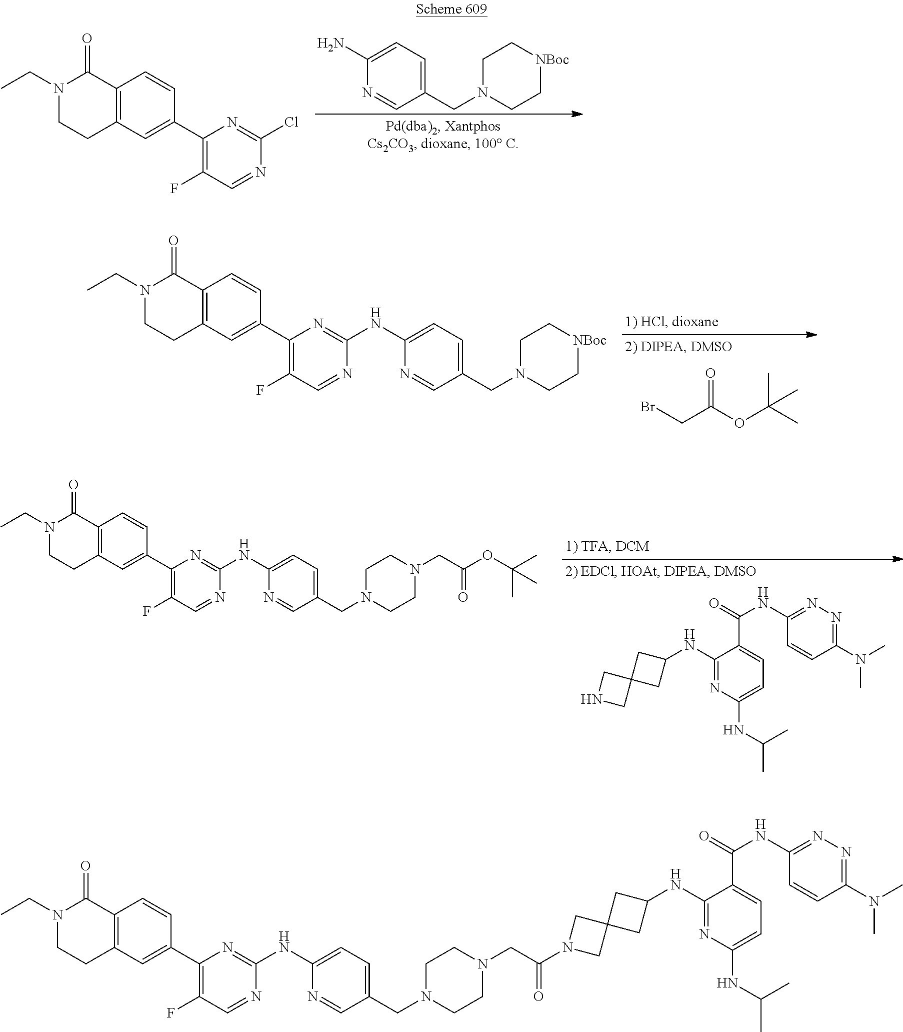

### Step 1. Synthesis of tert-butyl 4-((6-((4-(2-ethyl-1-oxo-1,2,3,4-tetrahydroisoquinolin-6-yl)-5-fluoropyrimidin-2-yl)amino)pyridin-3-yl)methyl)piperazine-1-carboxylate

To a solution of 6-(2-chloro-5-fluoro-pyrimidin-4-yl)-2-ethyl-3,4-dihydroisoquinolin-1-one (1.00 g, 3.27 mmol) and tert-butyl 4-((6-aminopyridin-3-yl)methyl)piperazine-1-carboxylate (1.10 g, 3.76 mmol) in dioxane (20 mL) were added Cs₂CO₃ (2.00 g, 6.14 mmol), Pd(dba)₂ (200 mg, 347 μmol) and Xantphos (400 mg, 691 μmol) at rt under N₂. The mixture was stirred at 80 T for 12 h under N₂. After cooling down to rt, the mixture was poured into H₂O (100 mL) and extracted with ethyl acetate (50 mL×3). The combined organic phase was washed with brine (50 mL), dried with anhydrous Na₂SO₄, filtered, and concentrated under vacuum. The crude product was triturated with MeCN at rt for 1 h. The mixture was filtered and the filter cake was collected to provide the desired product (1.01 g, 54% yield) as a white solid. MS (ESI) m/z=376.1 [M+H]⁺.

### Step 2. Synthesis of 2-ethyl-6-(5-fluoro-2-((5-(piperazin-1-ylmethyl)pyridin-2-yl)amino)pyrimidin-4-yl)-3,4-dihydroisoquinolin-1(2H)-one

To a solution of tert-butyl 4-((6-((4-(2-ethyl-1-oxo-1,2,3,4-tetrahydroisoquinolin-6-yl)-5-fluoropyrimidin-2-yl)amino)pyridin-3-yl)methyl)piperazine-1-carboxylate (400 mg, 712 mol) was added HCl solution (2 M in dioxane, 4.0 mL). The mixture was stirred at rt for 2 h. The mixture was concentrated under vacuum to provide the desired product (328 mg, 88% yield) as a gray solid. ¹HNMR (400 MHz, D₂O) δ 8.61 (d, J=3.2 Hz, 1H), 8.37 (s, 1H), 8.30 (dd, J=2.0, 9.2 Hz, 1H), 7.87 (d, J=8.4 Hz, 1H), 7.80 (s, 1H), 7.72 (d, J=8.4 Hz, 1H), 7.49 (d, J=9.2 Hz, 1H), 4.32 (s, 2H), 3.56 (t, J=6.8 Hz, 2H), 3.53-3.47 (m, 4H), 3.46-3.40 (m, 6H), 2.96 (t, J=6.8 Hz, 2H), 1.11 (t, J=7.2 Hz, 3H). MS (ESI) m/z=462.2 [M+H]⁺.

The remaining steps were performed according to the procedures for preparing CPD-418 to provide the desired product (5.1 mg, 26% yield over 3 steps) as a yellow solid. MS (ESI) m/z=912.8 [M+H]⁺.

### Example 610. N-(6-(dimethylamino)pyridazin-3-yl)-2-((6-(2-(4-((6-((4-(2-ethyl-1-oxo-1,2,3,4-tetrahydroisoquinolin-6-yl)-5-fluoropyrimidin-2-yl)amino)pyridin-3-yl)methyl)piperazin-1-yl)acetamido)spiro[3.3]heptan-2-yl)amino)-6-(isopropylamino)nicotinamide (CPD-554)

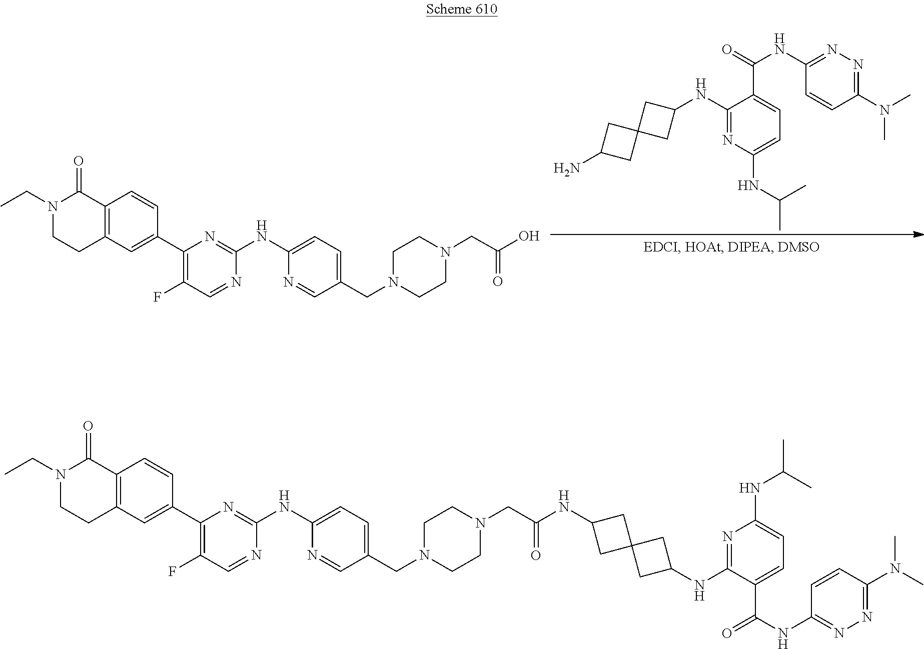 [SMILES: CCN1CCc2cc(-c3nc(Nc4ccc(CN5CCN(CC(=O)O)CC5)cn4)ncc3F)ccc2C1=O ; CC(C)Nc1ccc(C(=O)Nc2ccc(N(C)C)nn2)c(NC2CC3(CC(N)C3)C2)n1 ; CCN1CCc2cc(-c3nc(Nc4ccc(CN5CCN(CC(=O)NC6CC7(C6)CC(Nc6nc(NC(C)C)ccc6C(=O)Nc6ccc(N(C)C)nn6)C7)CC5)cn4)ncc3F)ccc2C1=O]

CPD-554 was synthesized following the standard procedure for preparing CPD-143 (6.4 mg, 45% yield) as a yellow solid. MS (ESI) m/z=926.8 [M+H]⁺.

### Example 611. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[3.5]nonan-7-yl)amino)-N-(6-(dimethylamino)pyridazin-3-yl)-6-(isopropylamino)nicotinamide (CPD-555)

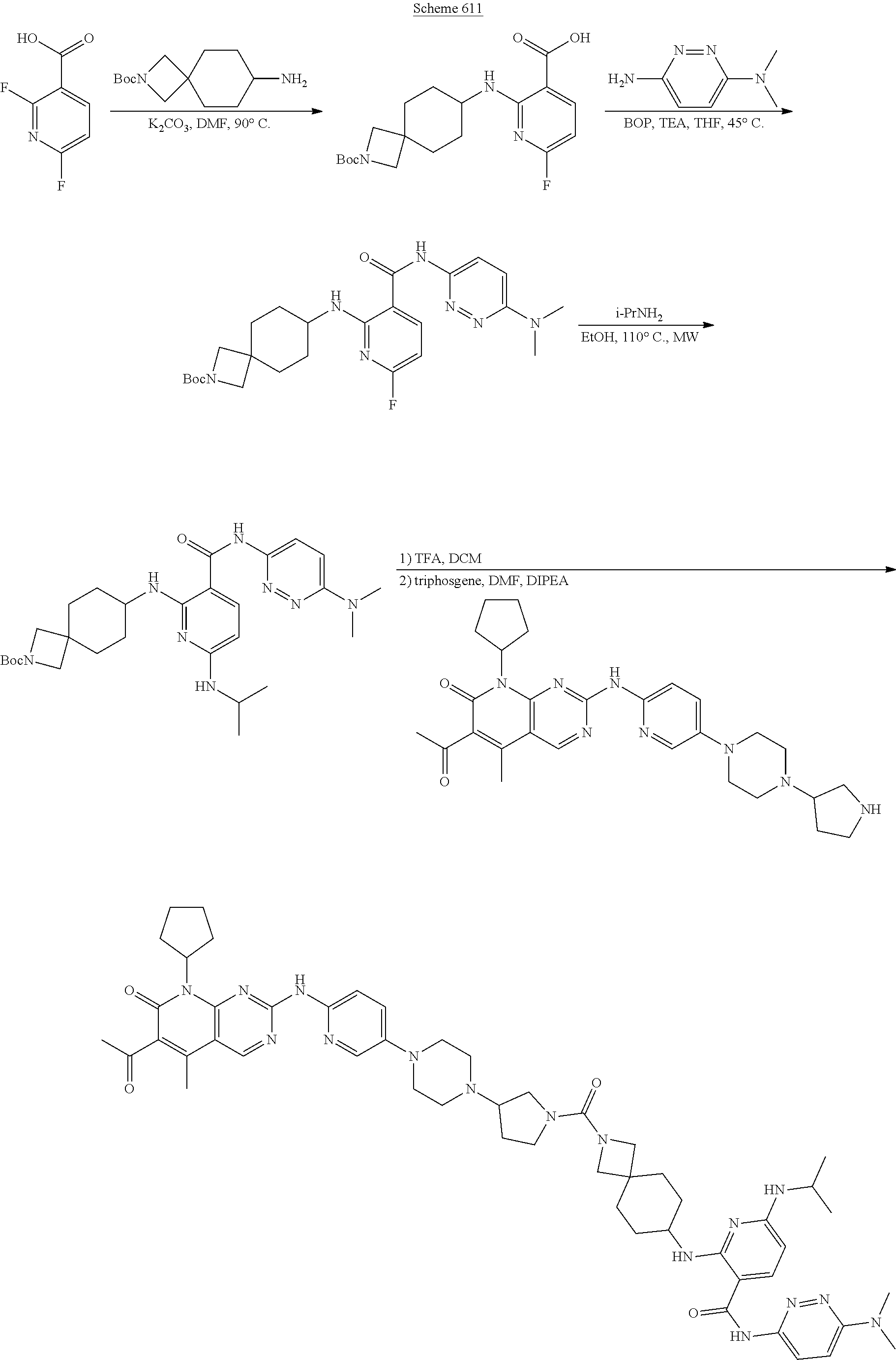

CPD-555 was synthesized following the standard procedures for preparing CPD-145 and CPD-469 (2.5 mg, 1% yield over 5 steps) as a yellow solid. MS (ESI) m/z=981.9 [M+H]⁺.

### Example 612. 2-((6-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)spiro[3.4]octan-2-yl)amino)-N-(6-(dimethylamino)pyridazin-3-yl)-6-(isopropylamino)nicotinamide (CPD-556)

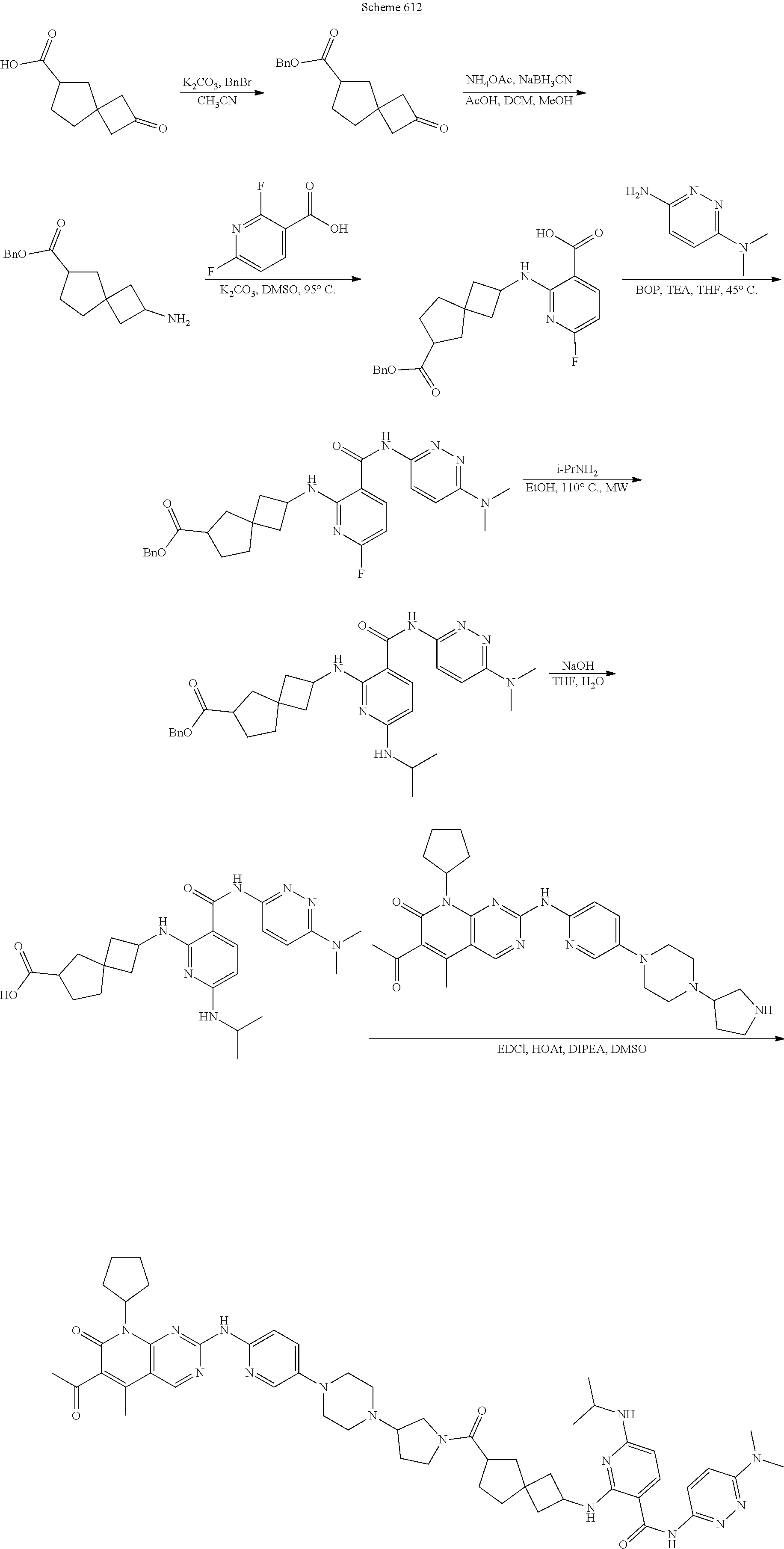

### Step 1. Synthesis of benzyl 2-oxospiro[3.4]octane-6-carboxylate

A mixture of 2-oxospiro[3.4]octane-7-carboxylic acid (80 mg, 0.48 mmol), K₂CO₃ (145 mg, 1.05 mmol) and BnBr (77 mg, 0.71 mmol) in CH₃CN (2 mL) was stirred at rt for 12 h. The mixture was diluted with water (5 mL) and extracted with EtOAc (3×10 mL). The combined organic phase was washed with brine, dried over Na₂SO₄, filtered, and concentrated to give the crude. The residue was purified by silica gel chromatography to provide the desired product (120 mg, 98%0 yield) as a white solid.

### Step 2. Synthesis of benzyl 2-aminospiro[3.4]octane-6-carboxylate

A mixture of benzyl 2-oxospiro[3.4]octane-6-carboxylate (120 mg, 0.46 mmol) and NH₄OAc (282 mg, 3.72 mmol) in dichloromethane (3 mL), MeOH (0.3 mL) and AcOH (0.1 mL) was stirred at rt for 2 h, followed by adding NaBH₃CN (146 mg, 2.32 mmol). The reaction mixture was stirred at rt for 12 h. The mixture was quenched with aq. NaHCO₃ solution (5 mL) and extracted with dichloromethane (3×20 mL). The combined organic phase was washed with brine, dried over Na₂SO₄, filtered and concentrated. The residue was purified by silica gel chromatography to provide the desired product (62 mg, 52% yield) as a white solid.

The remaining steps were performed according to the procedures for preparing CPD-82 to provide the desired product (4.2 mg, 2%8 yield over 5 steps) as a yellow solid. MS (ESI) m/z=966.5 [M+H]⁺.

### Example 613. N-(6-(dimethylamino)pyridazin-3-yl)-6-(isopropylamino)-2-((2-(2-(4-((6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)methyl)piperazin-1-yl)acetyl)-2-azaspiro[3.3]heptan-6-yl)amino)nicotinamide (CPD-557)

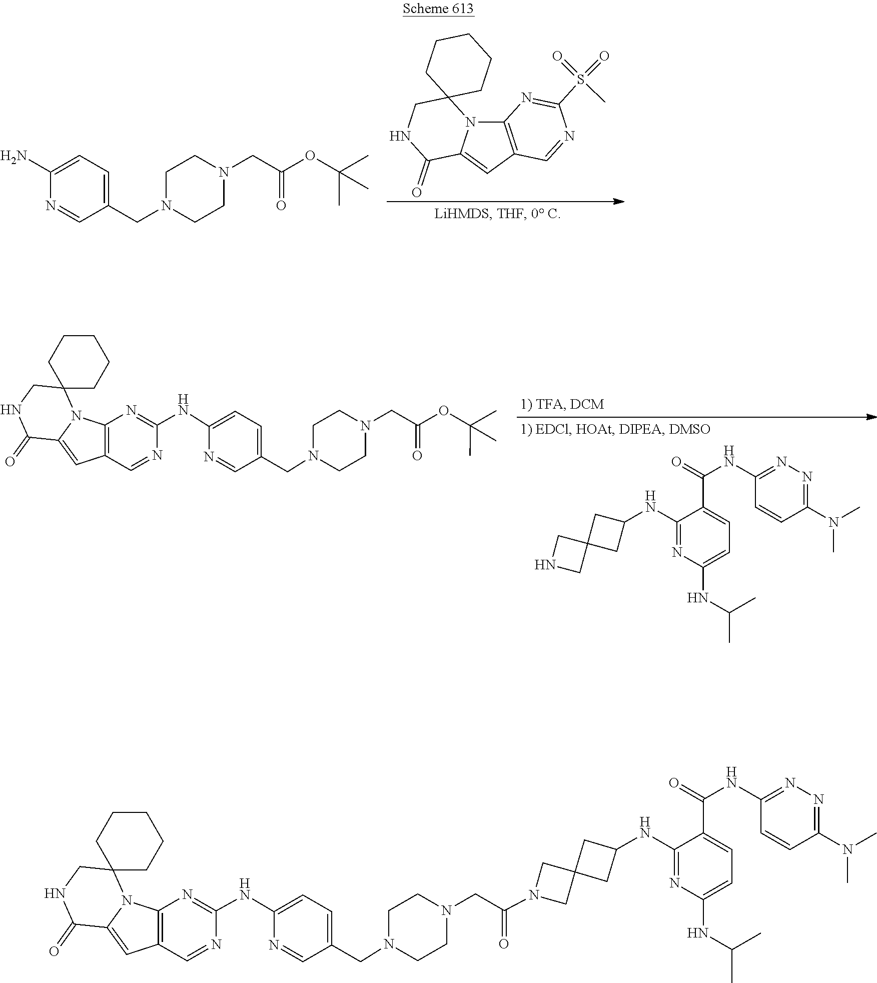

CPD-557 was synthesized following the standard procedures for preparing CPD-418 (1.1 mg, 2% yield over 3 steps) as a yellow solid. MS (ESI) m/z=897.6 [M+H]⁺.

### Example 614. N-(6-(dimethylamino)pyridazin-3-yl)-6-(isopropylamino)-2-((6-(2-(4-((6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)methyl)piperazin-1-yl)acetamido)spiro[3.3]heptan-2-yl)amino)nicotinamide (CPD-558)

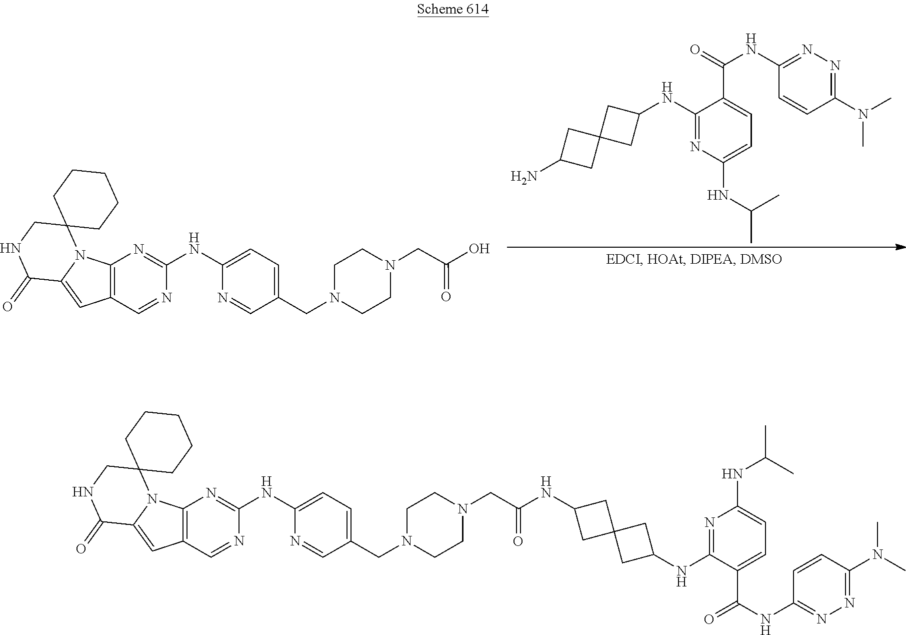 [SMILES: O=C(O)CN1CCN(Cc2ccc(Nc3ncc4cc5n(c4n3)C3(CCCCC3)CNC5=O)nc2)CC1 ; CC(C)Nc1ccc(C(=O)Nc2ccc(N(C)C)nn2)c(NC2CC3(CC(N)C3)C2)n1 ; CC(C)Nc1ccc(C(=O)Nc2ccc(N(C)C)nn2)c(NC2CC3(CC(NC(=O)CN4CCN(Cc5ccc(Nc6ncc7cc8n(c7n6)C6(CCCCC6)CNC8=O)nc5)CC4)C3)C2)n1]

CPD-558 was synthesized following the standard procedure for preparing CPD-143 (3.2 mg, 36% yield) as a yellow solid. MS (ESI) m/z=911.6 [M+H]⁺.

### Example 615. 2-((trans-4-(3-(4-(6-((8-cyclopentyl-6-(difluoromethyl)-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide (CPD-559)

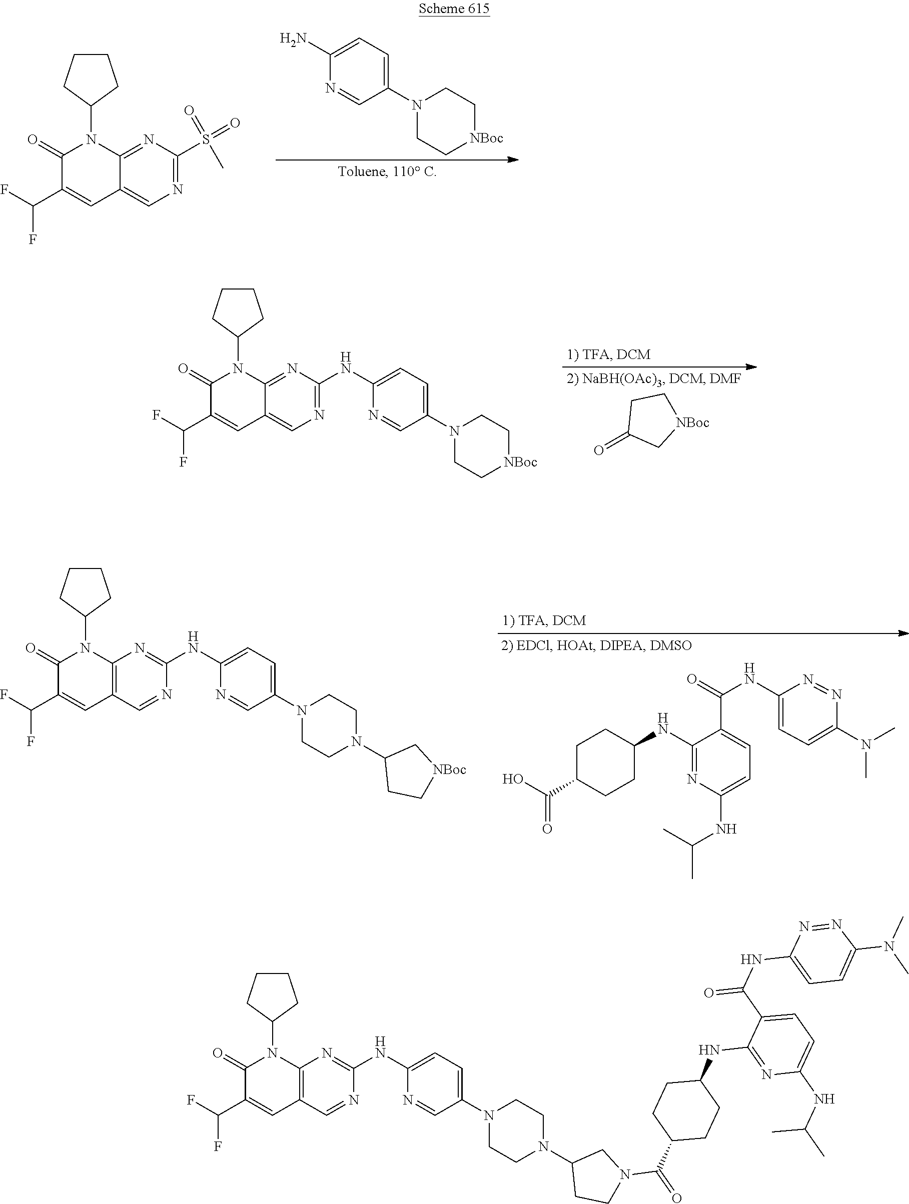

### Step 1. Synthesis of tert-butyl 4-(6-((8-cyclopentyl-6-(difluoromethyl)-7-oxo-7,8-dihydropyrido[2,3-d]pyrimidin-2-yl)amino)pyridin-3-yl)piperazine-1-carboxylate

To a solution of 8-cyclopentyl-6-(difluoromethyl)-2-methylsulfonyl-pyrido[2,3-d]pyrimidin-7-one (120 mg, 350 μmol) in toluene (4 mL) was added tert-butyl 4-(6-amino-3-pyridyl)piperazine-1-carboxylate (117 mg, 419 μmol). The mixture was stirred at 110° C. for 16 h. The mixture was concentrated under vacuum. The residue was triturated with CH₃CN (4 mL) at rt for 5 min to provide the desired product (20.0 mg, 11% yield) as a yellow solid. MS (ESI) m/z=542.3 [M+H]⁺.

### Step 2. Synthesis of 8-cyclopentyl-6-(difluoromethyl)-2-((5-(piperazin-1-yl)pyridin-2-yl)amino)pyrido[2,3-d]pyrimidin-7(8H)-one

To a solution of tert-butyl 4-[6-[[8-cyclopentyl-6-(difluoromethyl)-7-oxo-pyrido[2,3-d]pyrimidin-2-yl]amino]-3-pyridyl]piperazine-1-carboxylate (20.0 mg, 36.9 μmol) in dichloromethane (1 mL) was added TFA (0.5 mL) at rt. The mixture was stirred at rt for 1 h. The reaction mixture was concentrated under vacuum to provide the desired product (15.0 mg, 87% yield) as a yellow solid. ¹HNMR (400 MHz, MeOH-d₄) δ 9.08 (s, 1H), 8.28-8.24 (m, 2H), 7.99 (d, J=2.8 Hz, 1H), 7.59 (d, J=9.6 Hz, 1H), 6.84 (t, J=54.8 Hz, 1H), 6.07-5.99 (m, 1H), 3.58-3.55 (m, 4H), 3.47-3.44 (m, 4H), 2.37-2.30 (m, 2H), 2.15-2.10 (m, 2H), 1.93-1.87 (m, 2H), 1.75-1.68 (m, 2H). MS (ESI) m/z=442.2 [M+H]⁺.

The remaining steps were performed according to the procedures for preparing CPD-510 to provide the desired product (2.4 mg, 13% yield over 3 steps) as a yellow solid. MS (ESI) m/z=934.4 [M+H]⁺.

### Example 616. 2-((trans-4-(3-(4-(6-((6-acetyl-8-cyclopentyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(isopropylamino)nicotinamide (CPD-560)

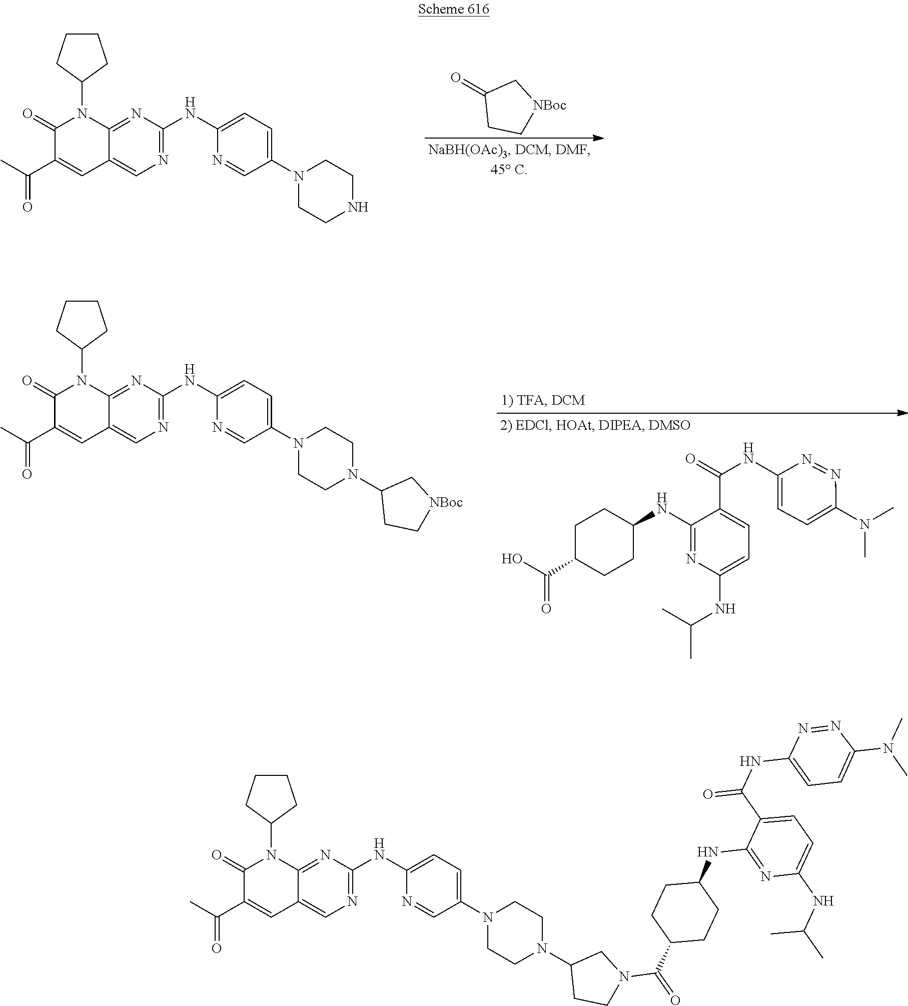 [SMILES: CC(=O)c1cc2cnc(Nc3ccc(N4CCNCC4)cn3)nc2n(C2CCCC2)c1=O ; CC(C)(C)OC(=O)N1CCC(=O)C1 ; CC(=O)c1cc2cnc(Nc3ccc(N4CCN(C5CCN(C(=O)OC(C)(C)C)C5)CC4)cn3)nc2n(C2CCCC2)c1=O ; CC(C)Nc1ccc(C(=O)Nc2ccc(N(C)C)nn2)c(N[C@H]2CC[C@H](C(=O)O)CC2)n1 ; CC(=O)c1cc2cnc(Nc3ccc(N4CCN(C5CCN(C(=O)[C@H]6CC[C@H](Nc7nc(NC(C)C)ccc7C(=O)Nc7ccc(N(C)C)nn7)CC6)C5)CC4)cn3)nc2n(C2CCCC2)c1=O]

CPD-560 was synthesized following the standard procedures for preparing CPD-510 (2.0 mg, 13% yield over 3 steps) as a yellow solid. MS (ESI) m/z=926.4 [M+H]⁺.

### Example 617. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-2-azaspiro[4.4]nonan-7-yl)amino)-N-(6-(dimethylamino)pyridazin-3-yl)-6-(isopropylamino)nicotinamide (CPD-561)

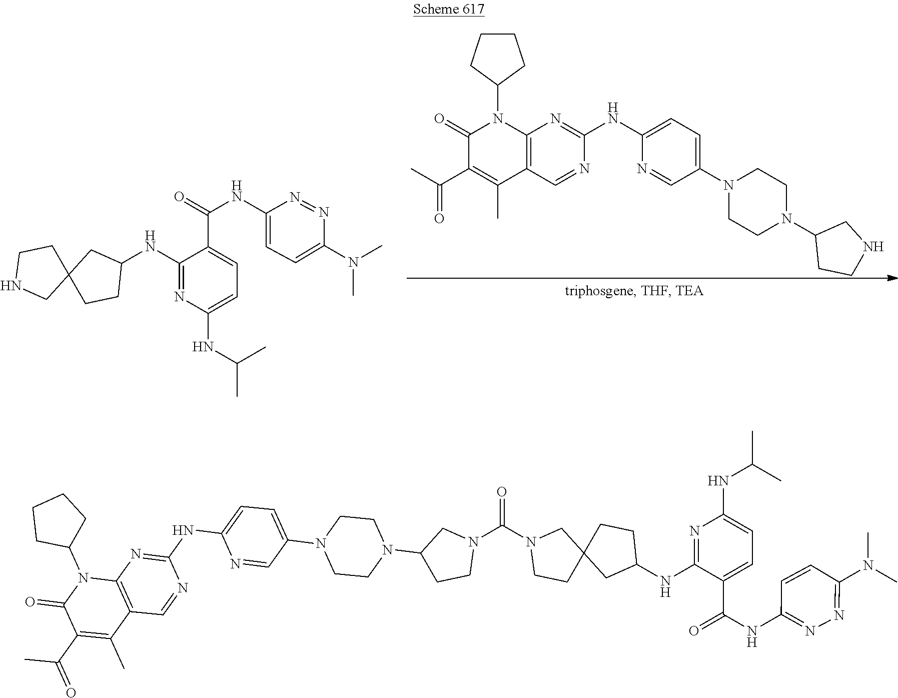 [SMILES: CC(C)Nc1ccc(C(=O)Nc2ccc(N(C)C)nn2)c(NC2CCC3(CCNC3)C2)n1 ; CC(=O)c1c(C)c2cnc(Nc3ccc(N4CCN(C5CCNC5)CC4)cn3)nc2n(C2CCCC2)c1=O ; CC(=O)c1c(C)c2cnc(Nc3ccc(N4CCN(C5CCN(C(=O)N6CCC7(CCC(Nc8nc(NC(C)C)ccc8C(=O)Nc8ccc(N(C)C)nn8)C7)C6)C5)CC4)cn3)nc2n(C2CCCC2)c1=O]

CPD-561 was synthesized following the standard procedure for preparing CPD-469 (3.5 mg, 20% yield) as a yellow solid. MS (ESI) m/z=981.9 [M+H]⁺.

### Example 618. 7-cyclopentyl-2-((5-((4-(2-((6-((3-((6-(dimethylamino)pyridazin-3-yl)carbamoyl)-6-(isopropylamino)pyridin-2-yl)amino)spiro[3.3]heptan-2-yl)amino)-2-oxoethyl)piperazin-1-yl)methyl)pyridin-2-yl)amino)-N,N-dimethyl-7H-pyrrolo[2,3-d]pyrimidine-6-carboxamide (CPD-562)

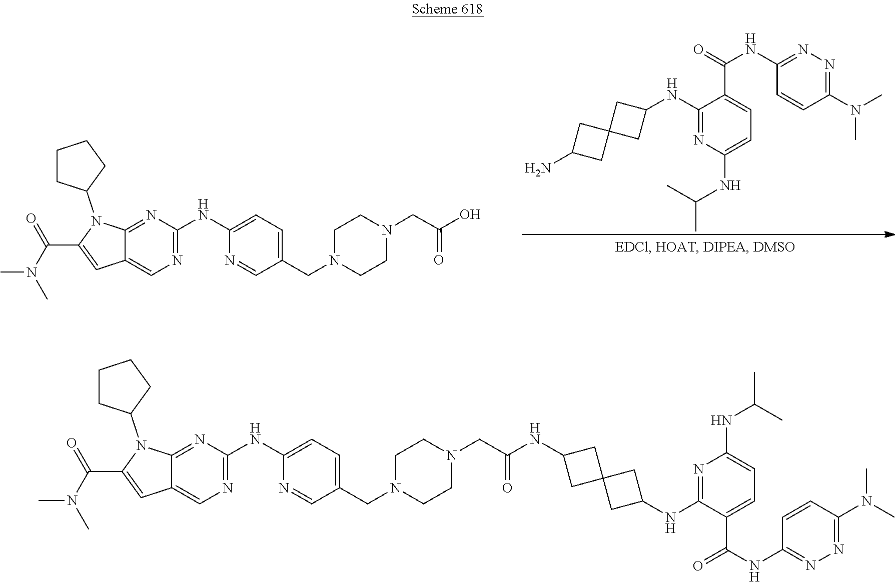

CPD-562 was synthesized following the standard procedure for preparing CPD-143 (2.0 mg, 18% yield) as a yellow solid. MS (ESI) m/z 913.8 [M+H]⁺.

### Example 619. 7-cyclopentyl-2-((5-((4-(2-(6-((3-((6-(dimethylamino)pyridazin-3-yl)carbamoyl)-6-(isopropylamino)pyridin-2-yl)amino)-2-azaspiro[3.3]heptan-2-yl)-2-oxoethyl)piperazin-1-yl)methyl)pyridin-2-yl)amino)-N,N-dimethyl-7H-pyrrolo[2,3-d]pyrimidine-6-carboxamide (CPD-563)

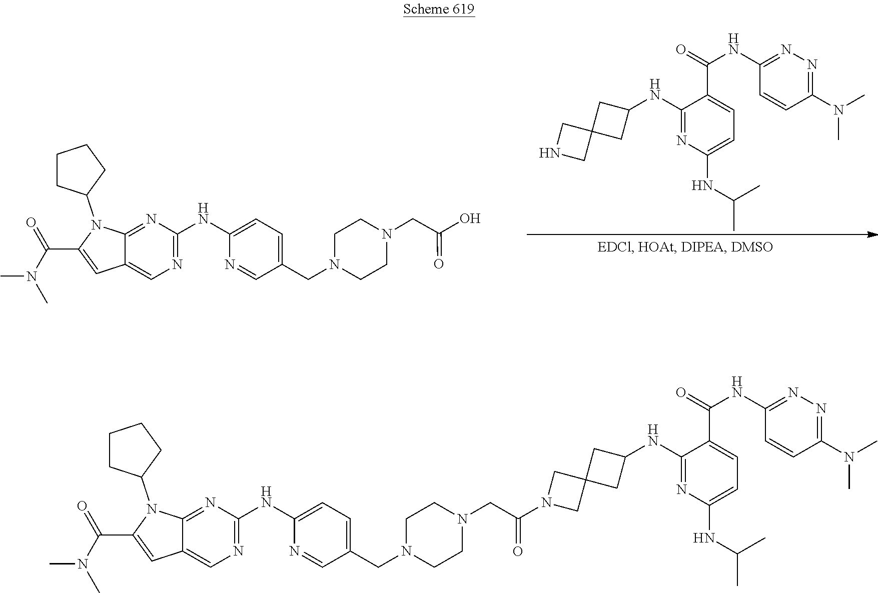

CPD-563 was synthesized following the standard procedure for preparing CPD-143 (2.0 mg, 20% yield) as a yellow solid. MS (ESI) m/z=899.9 [M+H]⁺.

### Example 620. 2-((1-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)piperidin-4-yl)amino)-N-(6,7-dihydro-5H-pyrrolo[1,2-a]imidazol-2-yl)-6-(isopropylamino)nicotinamide (CPD-564)

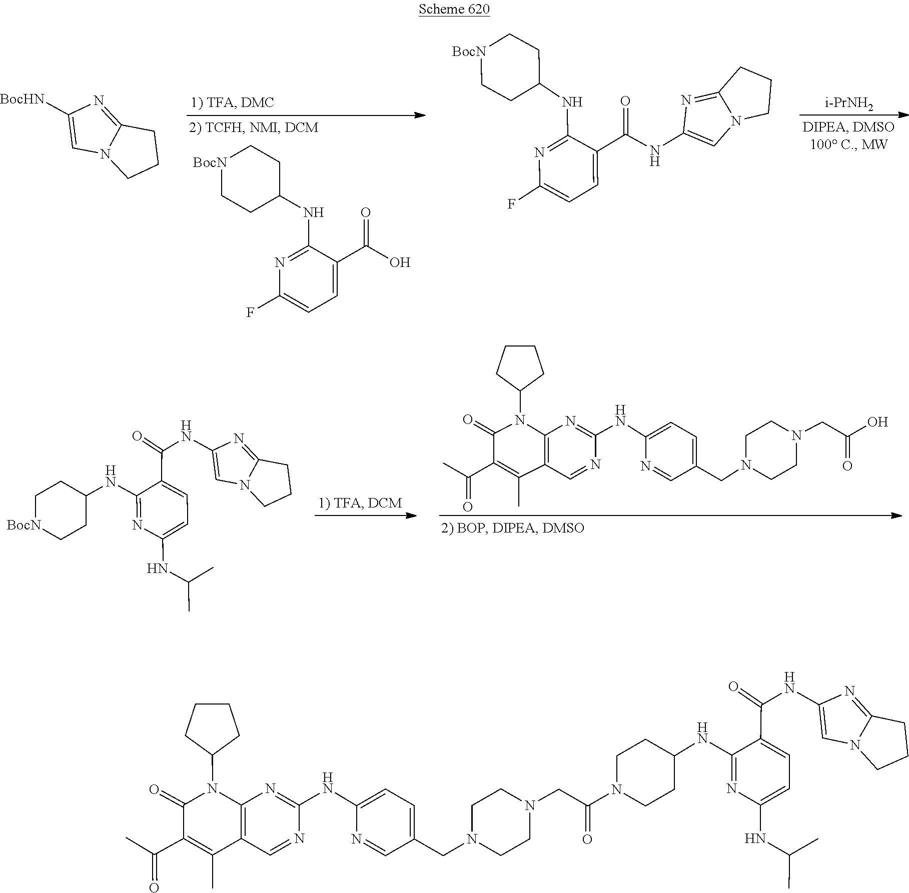

CPD-564 was synthesized following the standard procedures for preparing CPD-144 (5.0 mg, 14% yield over 5 steps) as a yellow solid. MS (ESI) m/z=885.8 [M+H]⁺.

### Example 621. 2-((2-(2-(7-(6-((6-acetyl-8-cyclopentyl-5-methyl-7-oxo-7,8-dihydropyrido[2,3-d]pyrimidin-2-yl)amino)pyridin-3-yl)-2,7-diazaspiro[4.4]nonan-2-yl)acetyl)-2-azaspiro[3.3]heptan-6-yl)amino)-N-(6,7-dihydro-5H-pyrrolo[1,2-a]imidazol-2-yl)-6-(isopropylamino)nicotinamide (CPD-565)

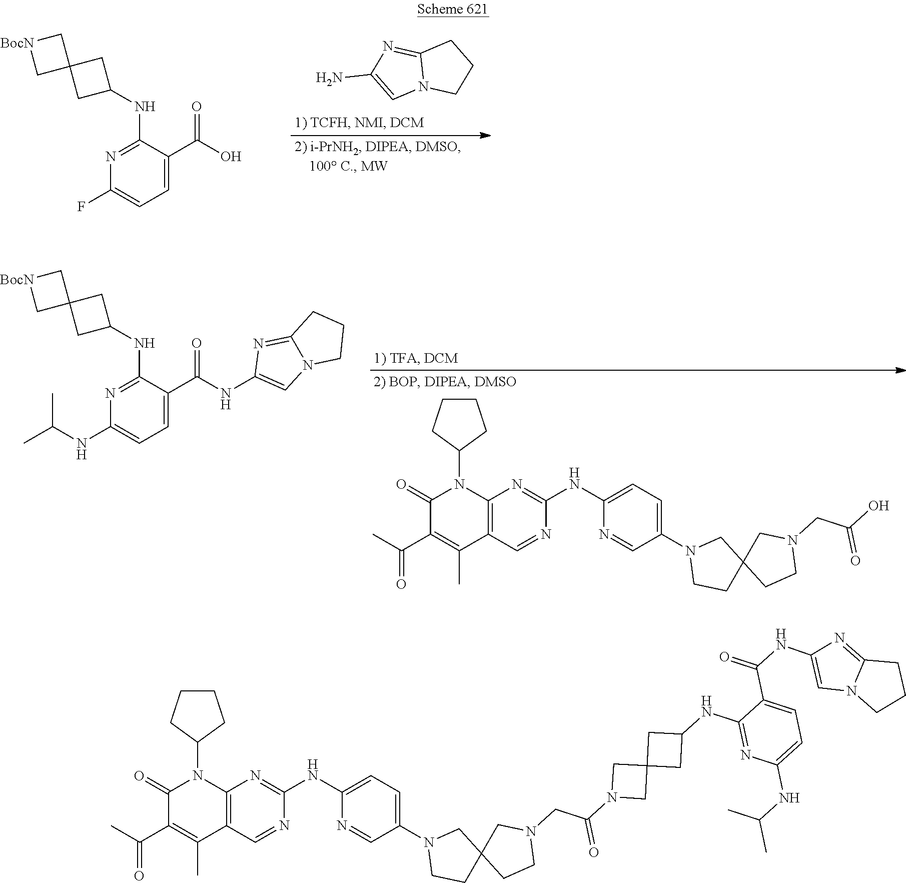 [SMILES: CC(C)(C)OC(=O)N1CC2(CC(Nc3nc(F)ccc3C(=O)O)C2)C1 ; Nc1cn2c(n1)CCC2 ; CC(C)Nc1ccc(C(=O)Nc2cn3c(n2)CCC3)c(NC2CC3(C2)CN(C(=O)OC(C)(C)C)C3)n1 ; CC(=O)c1c(C)c2cnc(Nc3ccc(N4CCC5(CCN(CC(=O)O)C5)C4)cn3)nc2n(C2CCCC2)c1=O ; CC(=O)c1c(C)c2cnc(Nc3ccc(N4CCC5(CCN(CC(=O)N6CC7(CC(Nc8nc(NC(C)C)ccc8C(=O)Nc8cn9c(n8)CCC9)C7)C6)C5)C4)cn3)nc2n(C2CCCC2)c1=O]

CPD-565 was synthesized following the standard procedures for preparing CPD-144 (2.0 mg, 4% yield over 4 steps) as a yellow solid. MS (ESI) m/z=923.7 [M+H]⁺.

### Example 622. 2-((6-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)spiro[3.3]heptan-2-yl)amino)-N-(6,7-dihydro-5H-pyrrolo[1,2-a]imidazol-2-yl)-6-(isopropylamino)nicotinamide (CPD-566)

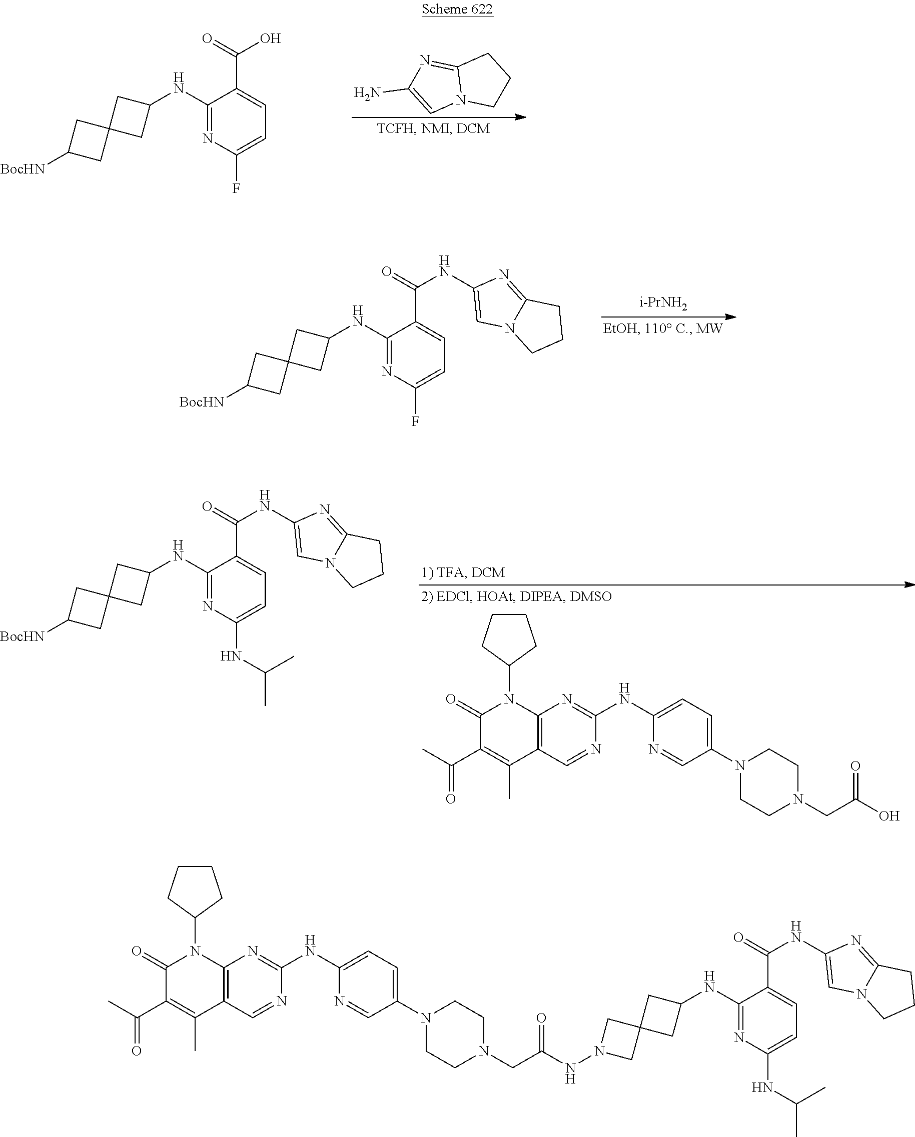

CPD-566 was synthesized following the standard procedures for preparing CPD-144 (4.1 mg, 4% yield over 4 steps) as a yellow solid. MS (ESI) m/z=897.8 [M+H]⁺.

### Example 623. 2-((2-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)-4,4,4-trifluorobutanoyl)-2-azaspiro[3.3]heptan-6-yl)amino)-N-(6,7-dihydro-5H-pyrrolo[1,2-a]imidazol-2-yl)-6-((methyl-d₃)amino)nicotinamide (CPD-567)

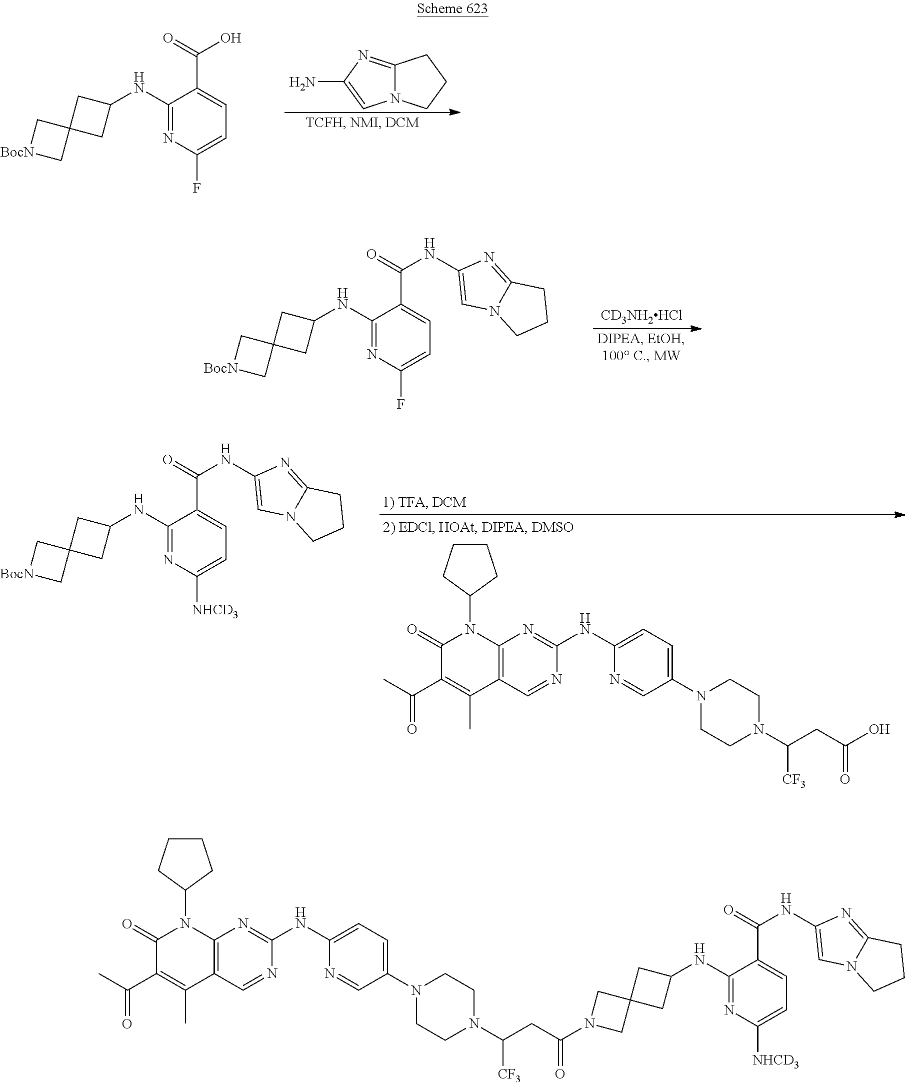

CPD-567 was synthesized following the standard procedures for preparing CPD-144 (1.4 mg, 1% yield over 4 steps) as a yellow solid. MS (ESI) m/z=940.9 [M+H]⁺.

### Example 624. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6,7-dihydro-5H-pyrrolo[1,2-a]imidazol-2-yl)-6-(isopropylamino)nicotinamide (CPD-568)

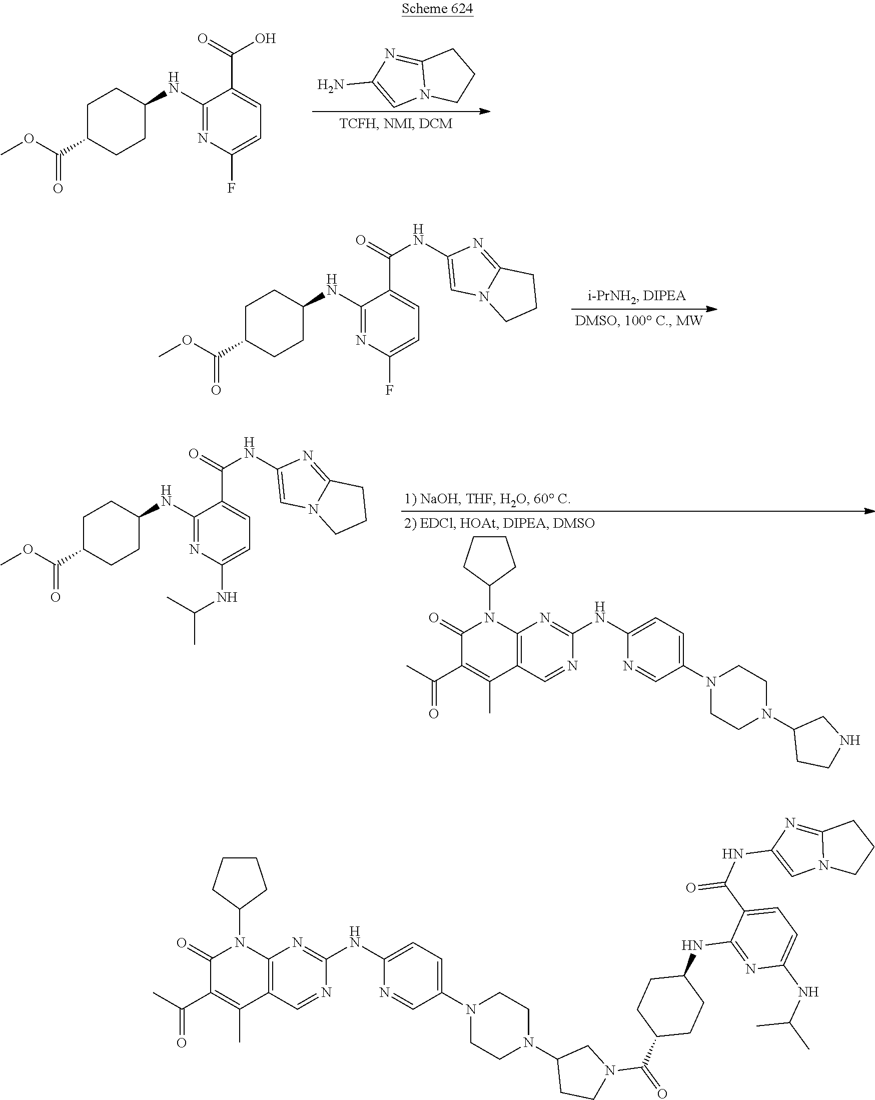 [SMILES: COC(=O)[C@H]1CC[C@H](Nc2nc(F)ccc2C(=O)O)CC1 ; Nc1cn2c(n1)CCC2 ; COC(=O)[C@H]1CC[C@H](Nc2nc(F)ccc2C(=O)Nc2cn3c(n2)CCC3)CC1 ; COC(=O)[C@H]1CC[C@H](Nc2nc(NC(C)C)ccc2C(=O)Nc2cn3c(n2)CCC3)CC1 ; CC(=O)c1c(C)c2cnc(Nc3ccc(N4CCN(C5CCNC5)CC4)cn3)nc2n(C2CCCC2)c1=O ; CC(=O)c1c(C)c2cnc(Nc3ccc(N4CCN(C5CCN(C(=O)[C@H]6CC[C@H](Nc7nc(NC(C)C)ccc7C(=O)Nc7cn8c(n7)CCC8)CC6)C5)CC4)cn3)nc2n(C2CCCC2)c1=O]

CPD-568 was synthesized following the standard procedures for preparing CPD-82 (0.9 mg, 1% yield over 4 steps) as a yellow solid. MS (ESI) m/z=925.8 [M+H]⁺.

### Example 625. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6,7-dihydro-5H-pyrrolo[1,2-a]imidazol-2-yl)-6-((methyl-d₃)amino)nicotinamide (CPD-569)

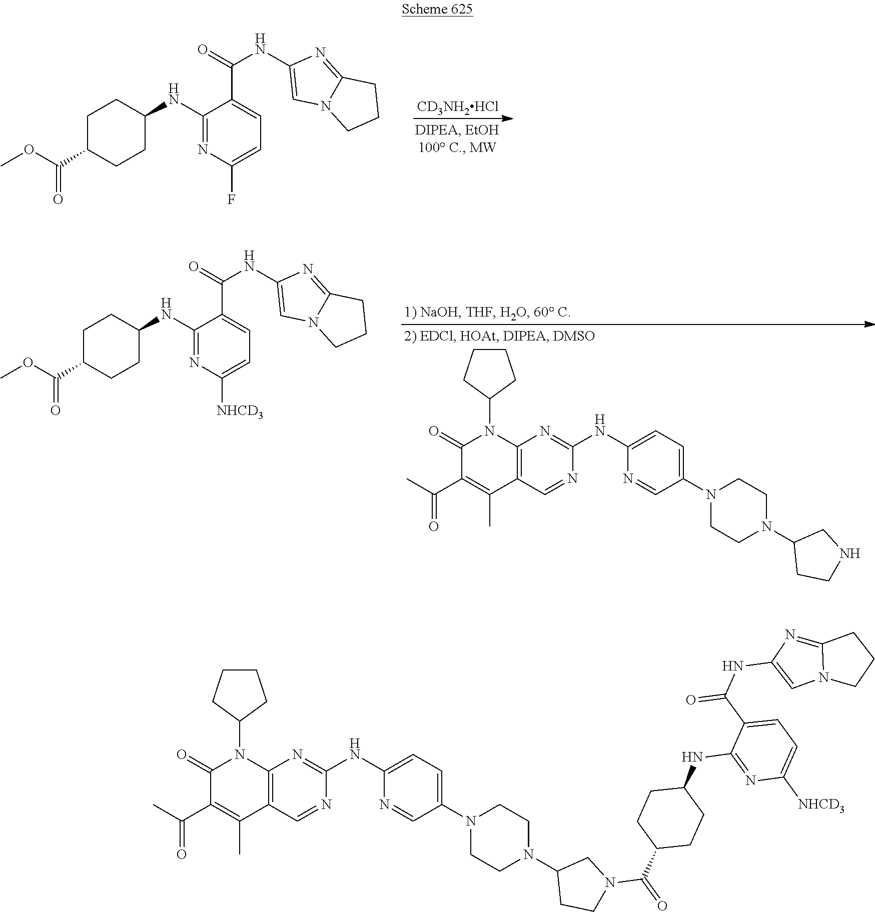

CPD-569 was synthesized following the standard procedures for preparing CPD-82 (1.6 mg, 5% yield over 3 steps) as a yellow solid. MS (ESI) m/z=900.8 [M+H]⁺.

### Example 626. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-6-azaspiro[3.4]octan-2-yl)amino)-N-(6-(dimethylamino)pyridazin-3-yl)-6-(isopropylamino)nicotinamide (CPD-570)

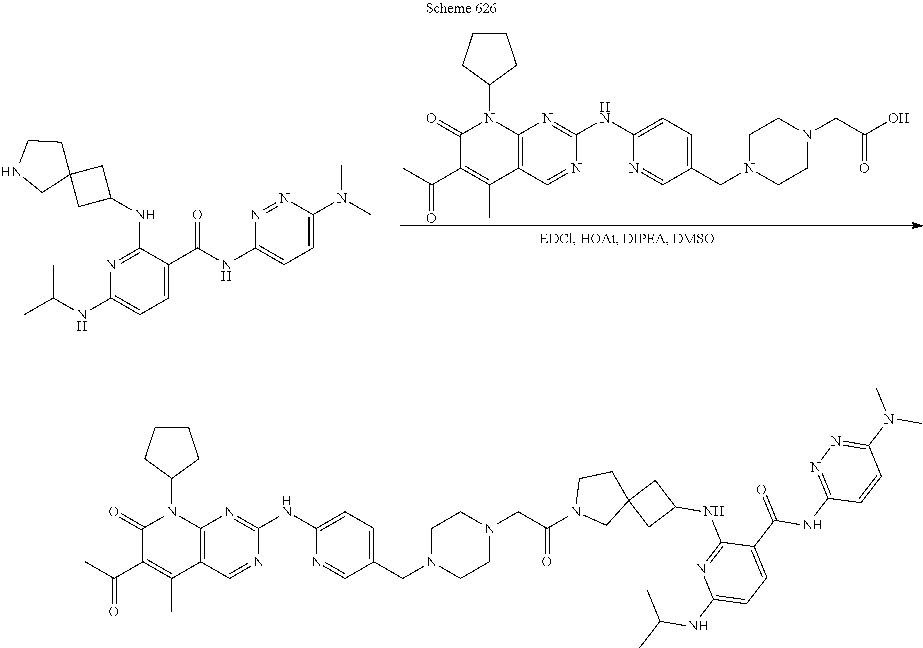 [SMILES: CC(C)Nc1ccc(C(=O)Nc2ccc(N(C)C)nn2)c(NC2CC3(CCNC3)C2)n1 ; CC(=O)c1c(C)c2cnc(Nc3ccc(CN4CCN(CC(=O)O)CC4)cn3)nc2n(C2CCCC2)c1=O ; CC(=O)c1c(C)c2cnc(Nc3ccc(CN4CCN(CC(=O)N5CCC6(CC(Nc7nc(NC(C)C)ccc7C(=O)Nc7ccc(N(C)C)nn7)C6)C5)CC4)cn3)nc2n(C2CCCC2)c1=O]

CPD-570 was synthesized following the standard procedure for preparing CPD-143 (3.0 mg, 27% yield) as a yellow solid. MS (ESI) m/z=926.8 [M+H]⁺.

### Example 627. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-(trifluoromethyl)nicotinamide (CPD-571)

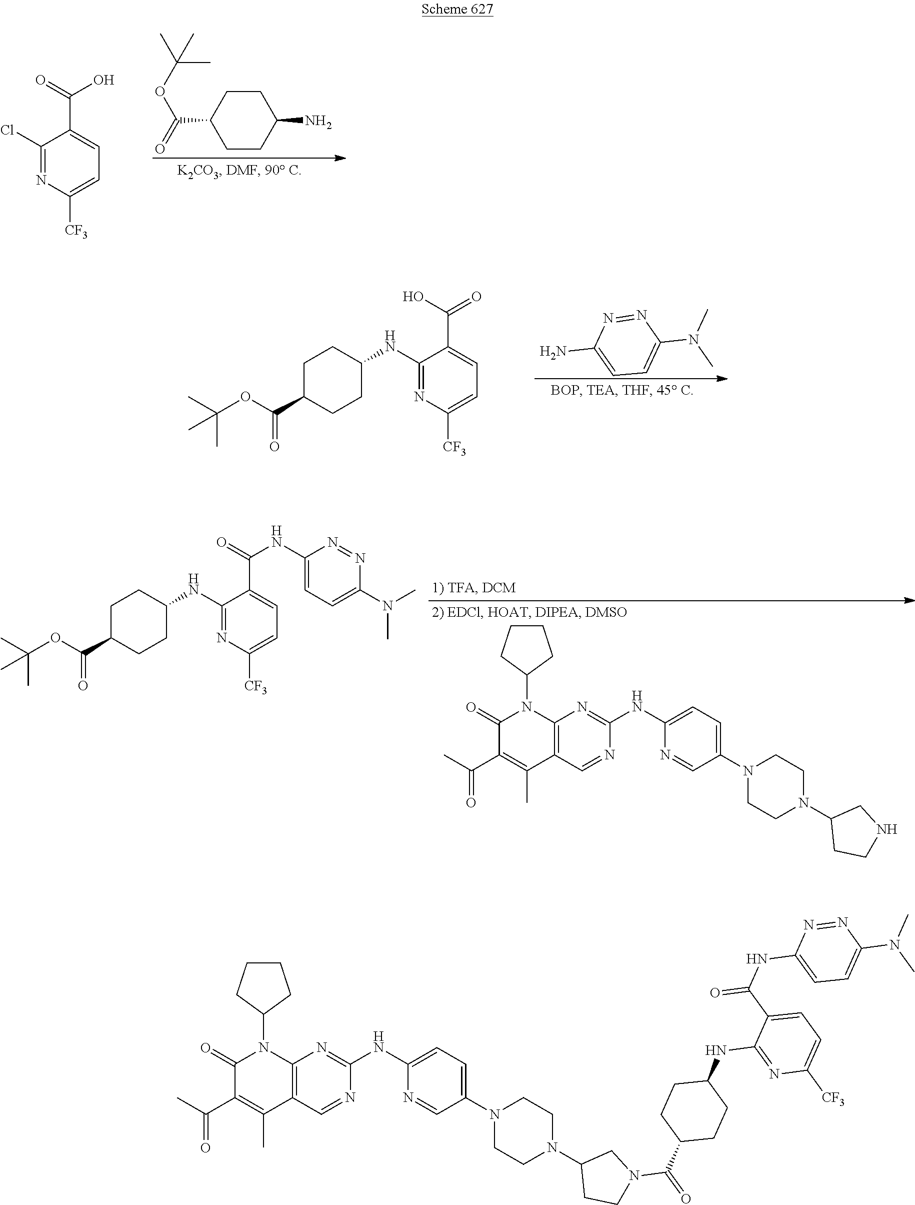

CPD-571 was synthesized following the standard procedure for preparing CPD-145 (3.0 mg, 7% yield over 4 steps) as a yellow solid. MS (ESI) m/z=951.7 [M+H]⁺.

### Example 628. 2-((4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-fluorocyclohexyl)amino)-N-(6-(dimethylamino)pyridazin-3-yl)-6-(isopropylamino)nicotinamide (CPD-572)

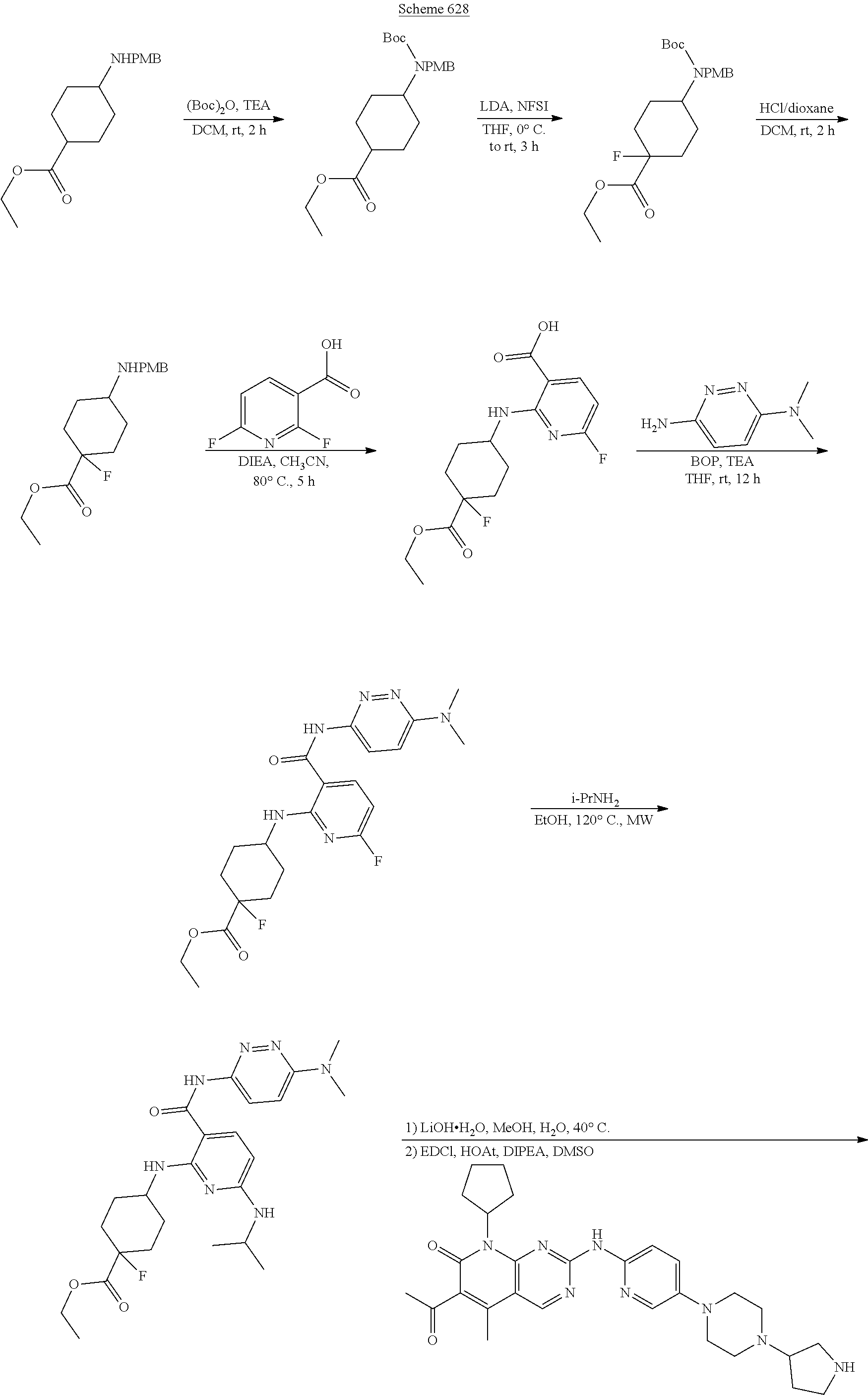

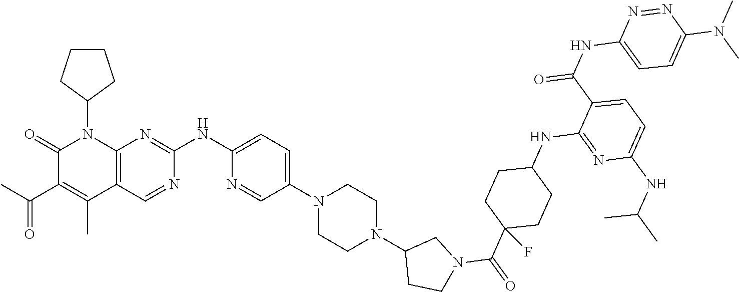 [SMILES: CC(=O)c1c(C)c2cnc(Nc3ccc(N4CCN(C5CCN(C(=O)C6(F)CCC(Nc7nc(NC(C)C)ccc7C(=O)Nc7ccc(N(C)C)nn7)CC6)C5)CC4)cn3)nc2n(C2CCCC2)c1=O]

### Step 1. Synthesis of ethyl 4-[tert-butoxycarbonyl-[(4-methoxyphenyl)methyl]amino]cyclohexanecarboxylate

To a mixture of ethyl 4-[(4-methoxyphenyl)methylamino]cyclohexanecarboxylate (33.0 g, 82.7 mmol) in dichloromethane (330 mL) were added (Boc)₂O (21.9 g, 100 mmol, 23 mL) and TEA (24.0 g, 237 mmol, 33.00 mL) at rt. The mixture was stirred at rt for 2 h. The resulting mixture was concentrated under vacuum and purified by silica gel chromatography (petroleum ether/ethyl acetate=4:1) to provide the desired product (18.0 g, 56% yield) as a yellow oil. ¹HNMR (400 MHz, CDCl₃) δ 7.15-7.13 (m, 2H), 6.85-6.81 (m, 2H), 4.31-4.27 (m, 2H), 4.15-4.07 (m, 2H), 3.80 (s, 3H), 2.19-2.16 (m, 1H) 1.59-1.41 (m, 9H), 1.23 (t, J=9.6 Hz, 3H). MS (ESI) m/z=392.2 [M+H]⁺.

### Step 2. Synthesis of ethyl 4-((tert-butoxycarbonyl)(4-methoxybenzyl)amino)-1-fluorocyclohexane-1-carboxylate

To a solution of ethyl 4-[tert-butoxycarbonyl-[(4-methoxyphenyl)methyl]amino]cyclohexanecarboxylate (15.0 g, 38.3 mmol) in THF (100 mL) was added LDA (2 M, 38.3 mL) dropwise at 0 [symbol] under N₂. After stirring at 0 [symbol] for 1 h, NFSI (24.1 g, 76.6 mmol) was added dropwise into the mixture at 0 CC. Then the mixture was stirred at rt for 2 h under N₂. The mixture was quenched with saturated NH₄Cl solution (400 mL) slowly. The aqueous phase was extracted with ethyl acetate (200 mL×3). The combined organic phase was washed with brine (500 mL), dried with anhydrous Na₂SO₄, filtered and concentrated. The residue was purified by column chromatography (SiO₂, petroleum ether/ethyl acetate=10:1 to 5/1) to provide the desired product (4.80 g, crude) as a yellow oil. MS (ESI) m/z=432.1 [M+Na]⁺.

### Step 3. Synthesis of ethyl 1-fluoro-4-((4-methoxybenzyl)amino)cyclohexane-1-carboxylate

To a solution of ethyl 4-[tert-butoxycarbonyl-[(4-methoxyphenyl)methyl]amino]-1-fluoro-cyclohexanecarboxylate (3.80 g, 9.28 mmol) in dichloromethane (20 mL) was added HCl solution (2 M in dioxane, 20 mL) at 0° C. The mixture was stirred at rt for 2 h. The mixture was concentrated and purified by prep-HPLC to provide the desired product (900 mg, 30% yield) as a yellow oil. ¹HNMR (400 MHz, DMSO-d₆) (7.25 (d, J=8.8 Hz, 2H), 6.85 (d, J=8.8 Hz, 2H), 4.14 (d, J=6.8 Hz, 2H), 3.73 (s, 3H), 3.68 (s, 2H), 2.49-2.44 (m, 1H), 1.97-1.89 (m, 2H), 1.86-1.80 (m, 2H), 1.79-1.63 (m, 2H), 1.33-1.28 (m, 2H), 1.20 (t, J=7.2 Hz, 3H). ¹⁹FNMR (400 MHz, DMSO-d₆) δ −164.49. MS (ESI) m/z=310.2 [M+H]⁺.

### Step 4. Synthesis of 2-((4-(ethoxycarbonyl)-4-fluorocyclohexyl)amino)-6-fluoronicotinic acid

To a solution of 2,6-difluoropyridine-3-carboxylic acid (926 mg, 5.82 mmol) in CH₃CN (8 mL) were added DIEA (1.13 g, 8.73 mmol) and ethyl 1-fluoro-4-[(4-methoxyphenyl)methylamino]cyclohexanecarboxylate (900 mg, 2.91 mmol). The mixture was stirred at 80° C. for 12 h. The reaction mixture was concentrated. The residue was re-dissolved in H₂O (50 mL) and adjusted pH to 4 with HCl (1 M), extracted with ethyl acetate (30 mL×3). The combined organic layers were washed with brine (30 mL), dried over Na₂SO₄, filtered and concentrated. The residue was purified by prep-HPLC and prep-TLC (SiO₂, petroleum ether/ethyl acetate=2:1) to provide the desired product (440 mg, 28% yield) as a yellow solid. ¹HNMR (400 MHz, CDCl₃) δ 8.20-8.36 (m, 1H), 8.03-7.99 (m, 1H), 6.13 (d, J=6.0 Hz, 1H), 4.29-4.23 (m, 2H), 4.18-4.06 (m, 1H), 2.20-1.98 (m, 6H), 1.75-1.60 (m, 2H), 1.32 (t, J=7.2 Hz, 3H). ¹FNMR (400 MHz, CDCl₃) δ −57.69, −166.97.

The remaining steps were performed according to the procedures for preparing CPD-82 to provide the desired product (7.7 mg, 12% yield over 4 steps) as a yellow solid. MS (ESI) m/z=958.8 [M+H]⁺.

### Example 629. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-cyclopropyl-N-(6-(dimethylamino)pyridazin-3-yl)nicotinamide (CPD-573)

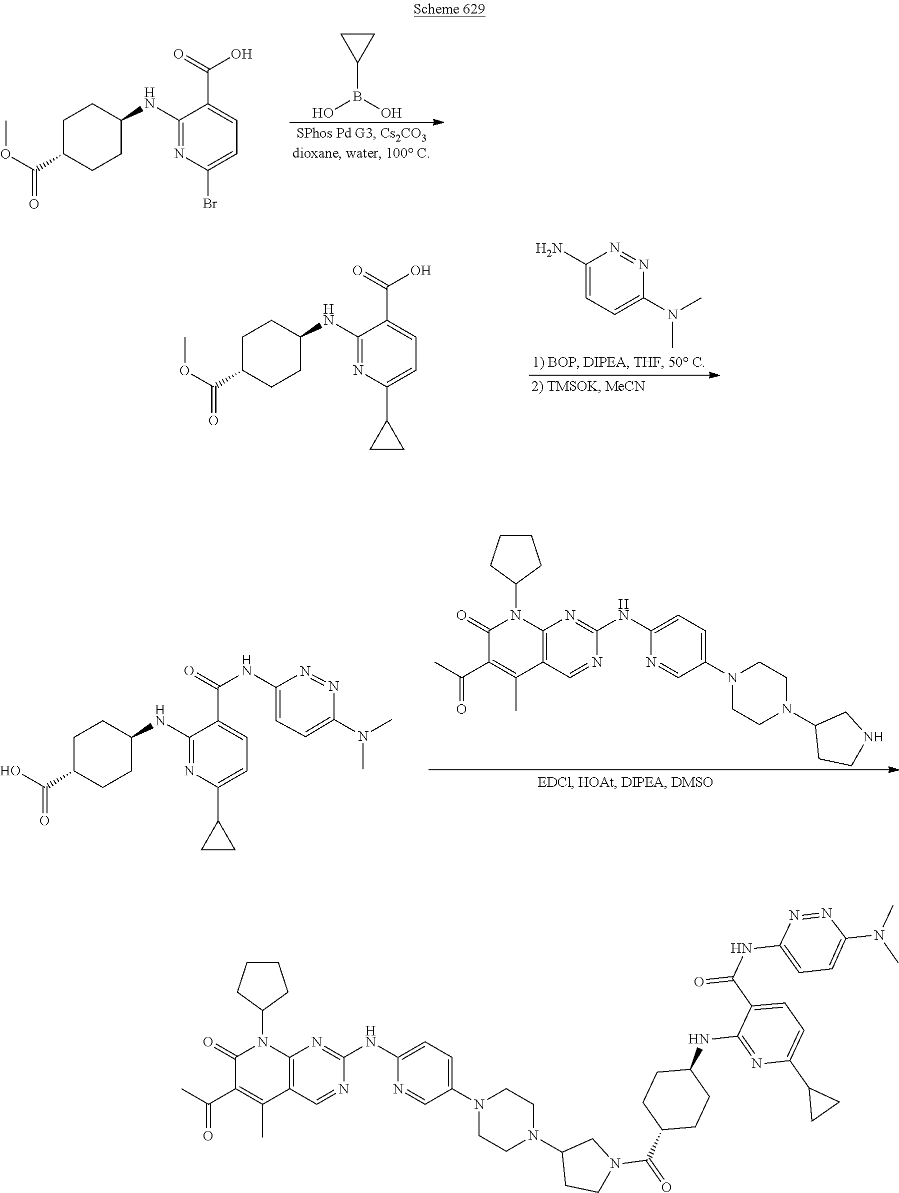

### Step 1. Synthesis of 6-cyclopropyl-2-((trans-4-(methoxycarbonyl)cyclohexyl)amino)nicotinic acid

To a solution of 6-bromo-2-((trans-4-(methoxycarbonyl)cyclohexyl)amino)nicotinic acid (100 mg, 279.96 μmol) in dioxane (5 mL) and water (1 mL) were added cyclopropylboronic acid (120.24 mg, 1.40 mmol), SPhos Pd G3 (10.92 mg, 14.00 μmol) and Cs₂CO₃ (182.43 mg, 559.91 μmol) at rt. The mixture was stirred at 100 [symbol] under inert atmosphere for 6 h. The reaction mixture was cooled to rt, filtered and concentrated. The resulting residue was purified by silica gel chromatography to give the desired product (70 mg, 79% yield) as a white solid. MS (ESI) m/z=319.6 [M+H]⁺.

### Step 2. Synthesis of methyl trans-4-((6-cyclopropyl-3-((6-(dimethylamino)pyridazin-3-yl)carbamoyl)pyridin-2-yl)amino)cyclohexane-1-carboxylate

To a solution of 6-cyclopropyl-2-((trans-4-(methoxycarbonyl)cyclohexyl)amino)nicotinic acid (30 mg, 94.23 mol) in THF (4 mL) were added N³,N³-dimethylpyridazine-3,6-diamine (13.02 mg, 94.23 μmol), BOP (62.51 mg, 141.35 μmol) and TEA (33.37 mg, 329.81 μmol). The mixture was stirred at 50° C. for 16 h. The reaction mixture was cooled to rt and concentrated. The resulting residue was purified by silica gel chromatography to give the desired product (33 mg, 80% yield) as a yellow solid. MS (ESI) m/z=439.5 [M+H]⁺.

### Step 3. Synthesis of trans-4-((6-cyclopropyl-3-((6-(dimethylamino)pyridazin-3-yl)carbamoyl)pyridin-2-yl)amino)cyclohexane-1-carboxylic acid

To a solution of methyl trans-4-((6-cyclopropyl-3-((6-(dimethylamino)pyridazin-3-yl)carbamoyl)pyridin-2-yl)amino)cyclohexane-1-carboxylate (20 mg, 45.61 μmol) in MeCN (5 mL) was added TMSOK (23.40 mg, 182.43 μmol) at rt. The reaction mixture was stirred at rt for 16 h. The mixture was quenched with water (20 mL), acidified with aq. HCl (1 M), and extracted with dichloromethane (3×20 mL). The combined organic layers were washed with saturated brine (15 mL), dried over anhydrous sodium sulfate, filtered, and concentrated. The resulting residue was purified by reverse-phase chromatography to give the desired product (5 mg, 26% yield) as a yellow solid. MS (ESI) m/z=425.5 [M+H]⁺.

### Step 4. Synthesis of 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-6-cyclopropyl-N-(6-(dimethylamino)pyridazin-3-yl)nicotinamide

To a mixture of trans-4-((6-cyclopropyl-3-((6-(dimethylamino)pyridazin-3-yl)carbamoyl)pyridin-2-yl)amino)cyclohexane-1-carboxylic acid (5 mg, 11.78 μmol), EDCI (3.39 mg, 17.67 μmol) and HOAt (2.40 mg, 17.67 μmol) in DMSO (1 mL) were added DIPEA (7.61 mg, 58.89 mol) and 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (6.09 mg, 11.78 μmol). The mixture was stirred at rt for 16 h, before it was purified by prep-HPLC to provide the desired product (2.2 mg, 20% yield) as a yellow solid. MS (ESI) m/z=923.8 [M+H]⁺.

### Example 630. 2-((cis-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-hydroxycyclohexyl)amino)-N-(6-(dimethylamino)pyridazin-3-yl)-6-(isopropylamino)nicotinamide (CPD-574) and 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)-4-hydroxycyclohexyl)amino)-N-(6-(dimethylamino)pyridazin-3-yl)-6-(isopropylamino)nicotinamide (CPD-575)

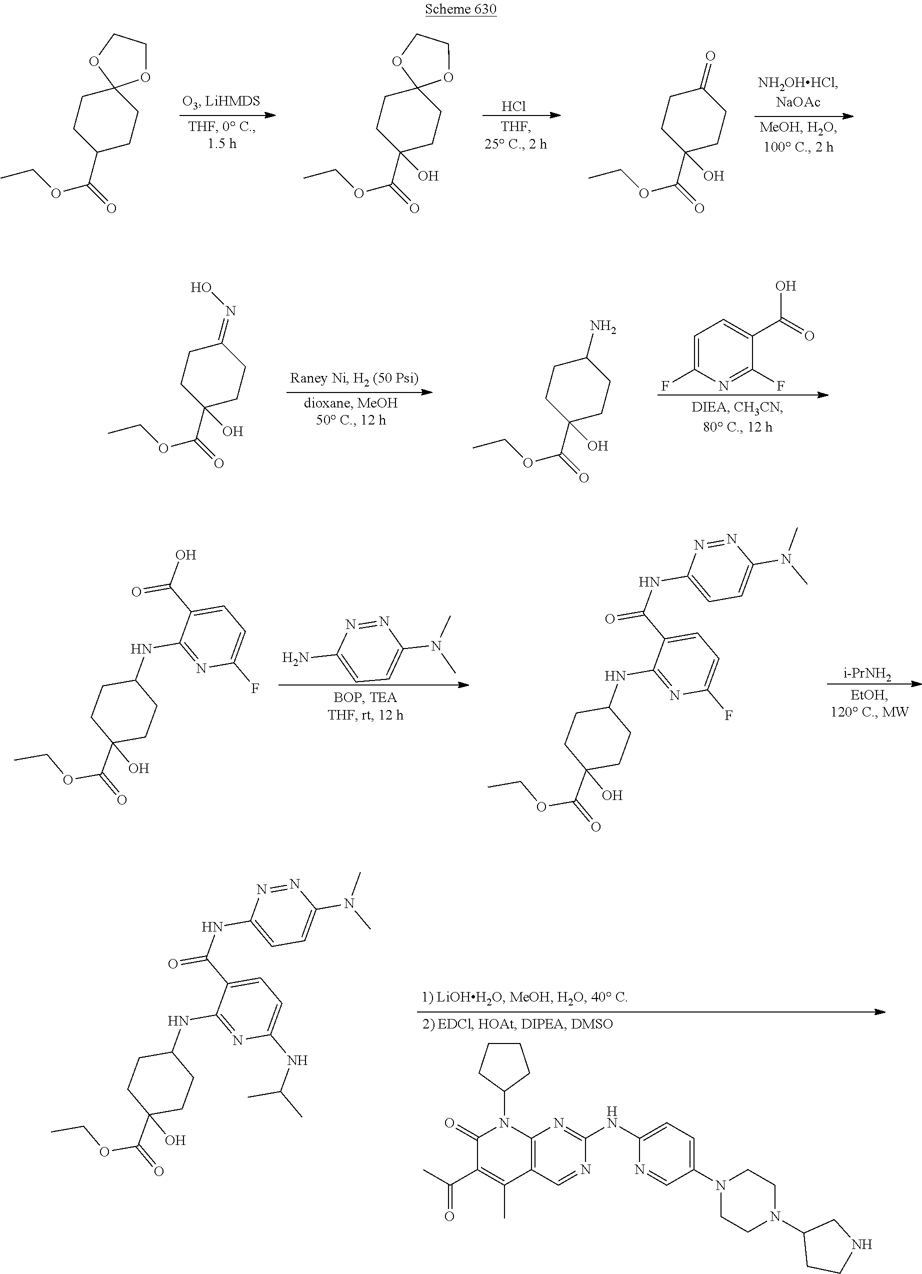

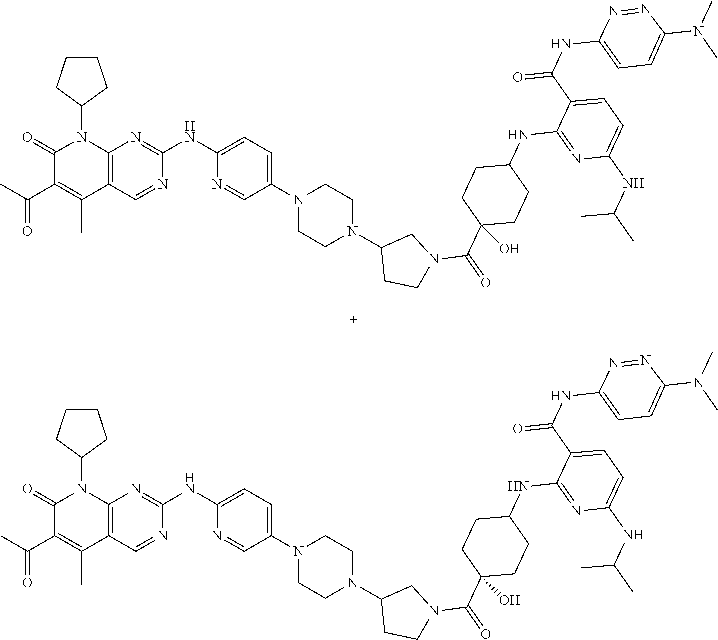

### Step 1. Synthesis of ethyl 8-hydroxy-1,4-dioxaspiro[4.5]decane-8-carboxylate

To a solution of ethyl 1,4-dioxaspiro[4.5]decane-8-carboxylate (2.00 g, 9.33 mmol) in THF (20 mL) was added LiHMDS (1 M, 14.0 mmol) at 0 [symbol] . The mixture was stirred at 0 [symbol] for 1 h. Then ozone was bubbled to the mixture for 0.5 h at 0 [symbol] . The resulting mixture was quenched with Na₂SO₃ solution (50 mL) and extracted with ethyl acetate (30 mL×3). The combined organic layer was washed with brine (30 mL), dried over Na₂SO₄, filtered and concentrated. The residue was purified by column chromatography (SiO₂, petroleum ether/ethyl acetate=20:1 to 10:1) to provide the desired product (1.60 g, 74% yield) as a yellow oil. ¹HNMR (400 MHz, CDCl₃) δ 4.24 (q, J=7.2 Hz, 2H), 3.98 (s, 3H), 3.94 (s, 1H), 2.97 (s, 1H), 2.17-2.06 (m, 2H), 2.04-1.92 (m, 2H), 1.70 (t, J=14.0 Hz, 4H), 1.30 (t, J=6.8 Hz, 3H).

The remaining steps were performed according to the procedures for preparing CPD-516 and CPD-82 to provide the desired products. The two isomers were separated by prep-HPLC. The putative cis isomer CPD-527 (13.0 mg, 2% yield over 8 steps) was got as a yellow solid. MS (ESI) m/z=956.8 [M+H]⁺. The putative trans isomer CPD-528 (4.8 mg, 1% yield over 8 steps) was got as a yellow solid. MS (ESI) m/z=956.9 [M+H]⁺.

### Example 631. 2-((trans-4-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)cyclohexyl)amino)-N-(6-(dimethylamino)pyridazin-3-yl)-6-methylnicotinamide (CPD-576)

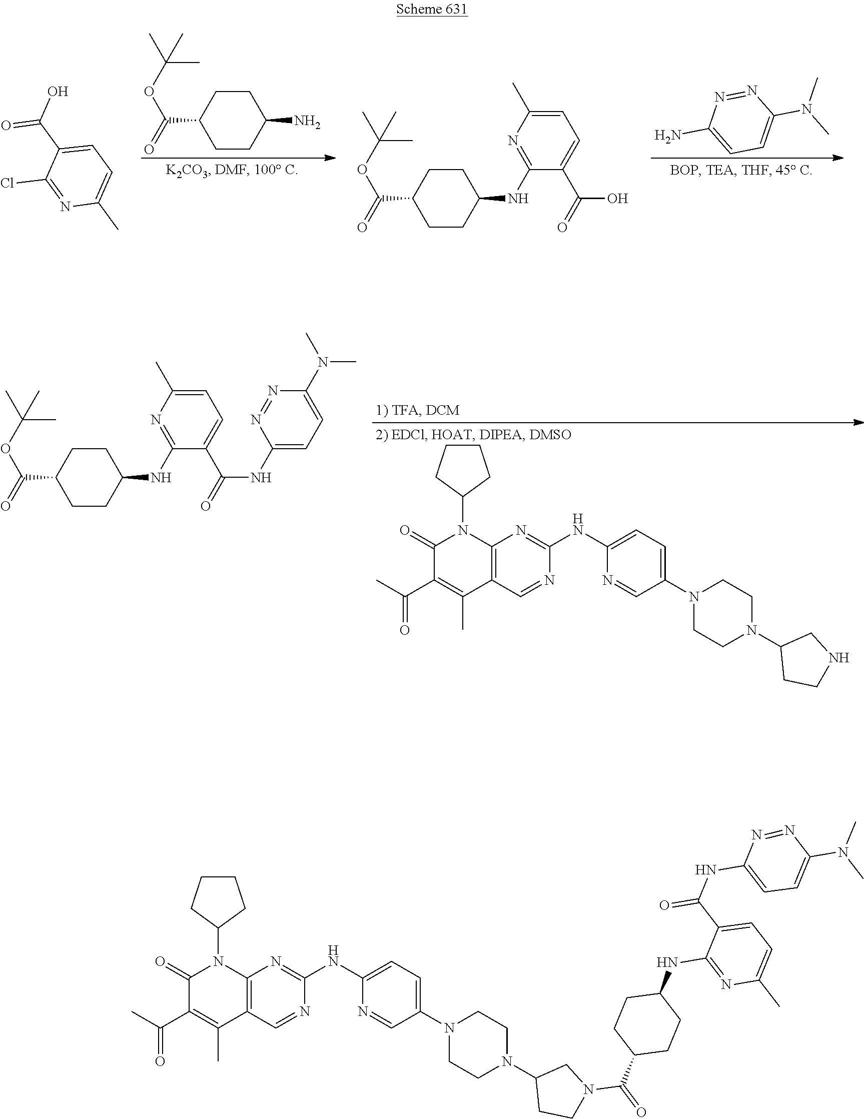

CPD-576 was synthesized following the standard procedures for preparing CPD-145 (2.0 mg, 4% yield over 4 steps) as a light-yellow solid. MS (ESI) m/z=897.8 [M+H]⁺.

### Example 632. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-ethylnicotinamide (CPD-577)

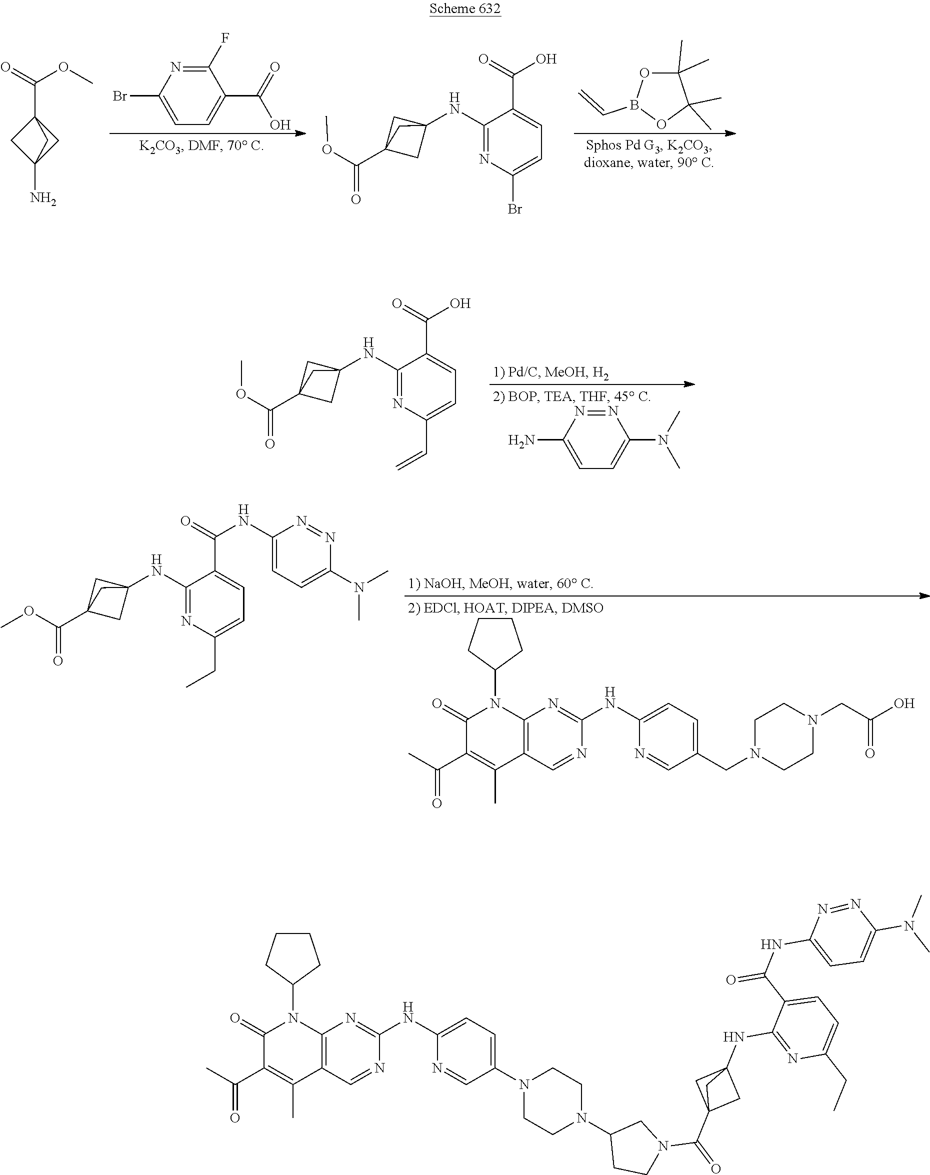

CPD-577 was synthesized following the standard procedures for preparing CPD-492 (3.0 mg, 10% yield over 6 steps) as a yellow solid. MS (ESI) m/z=895.8 [M+H]⁺.

### Example 633. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-ethylnicotinamide (CPD-578)

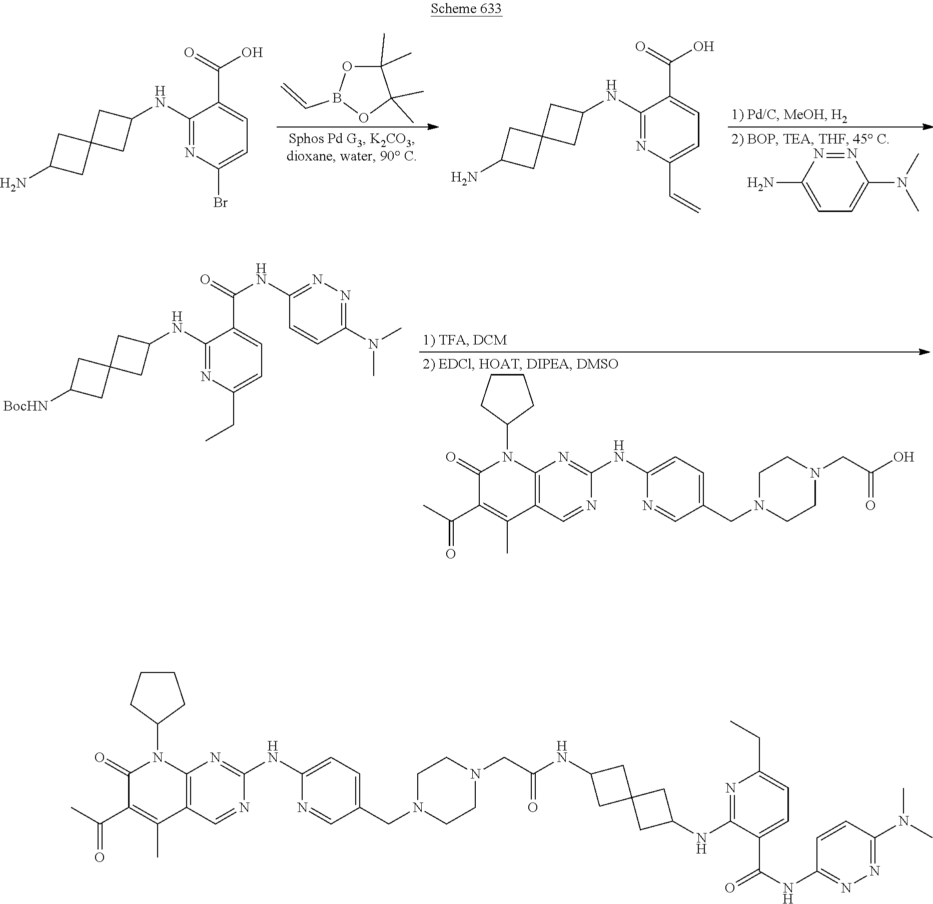

CPD-578 was synthesized following the standard procedures for preparing CPD-528 (2.6 mg, 3% yield over 5 steps) as a yellow solid. MS (ESI) m/z=897.8 [M+H]⁺.

### Example 634. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-6-isopropylnicotinamide (CPD-579)

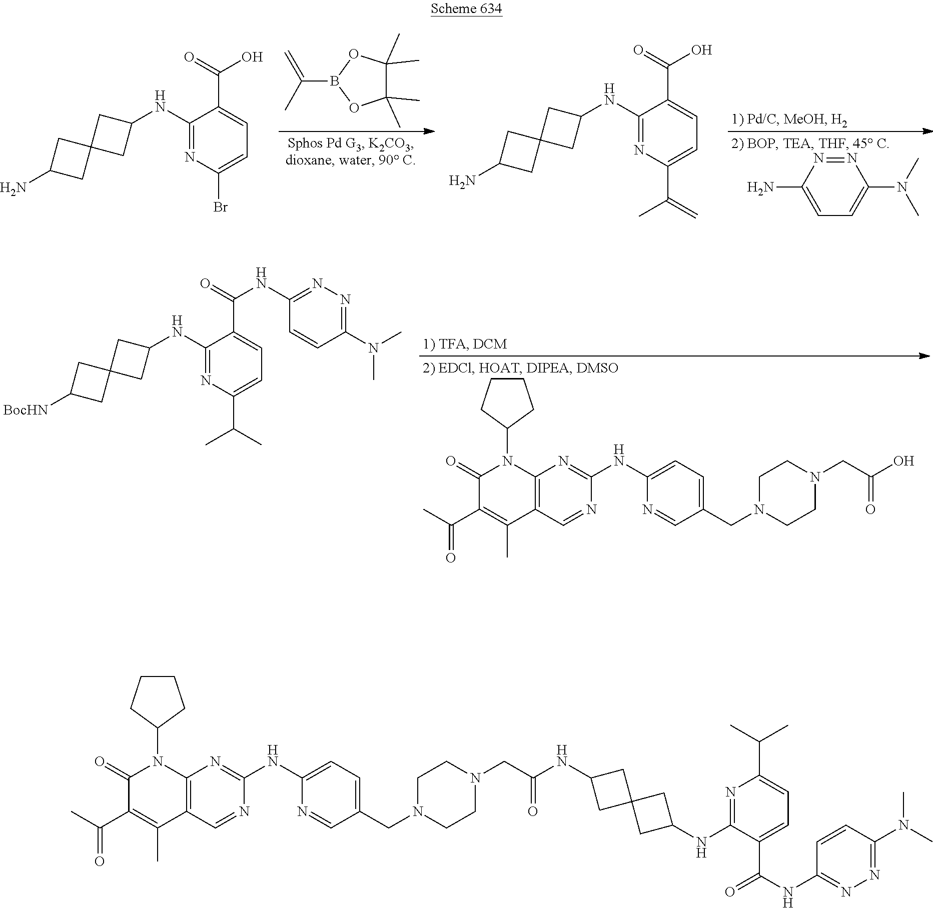

CPD-579 was synthesized following the standard procedures for preparing CPD-528 (3.0 mg, 3% yield over 5 steps) as a yellow solid. MS (ESI) m/z=911.8 [M±H]⁺.

### Example 635. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-isopropylnicotinamide (CPD-580)

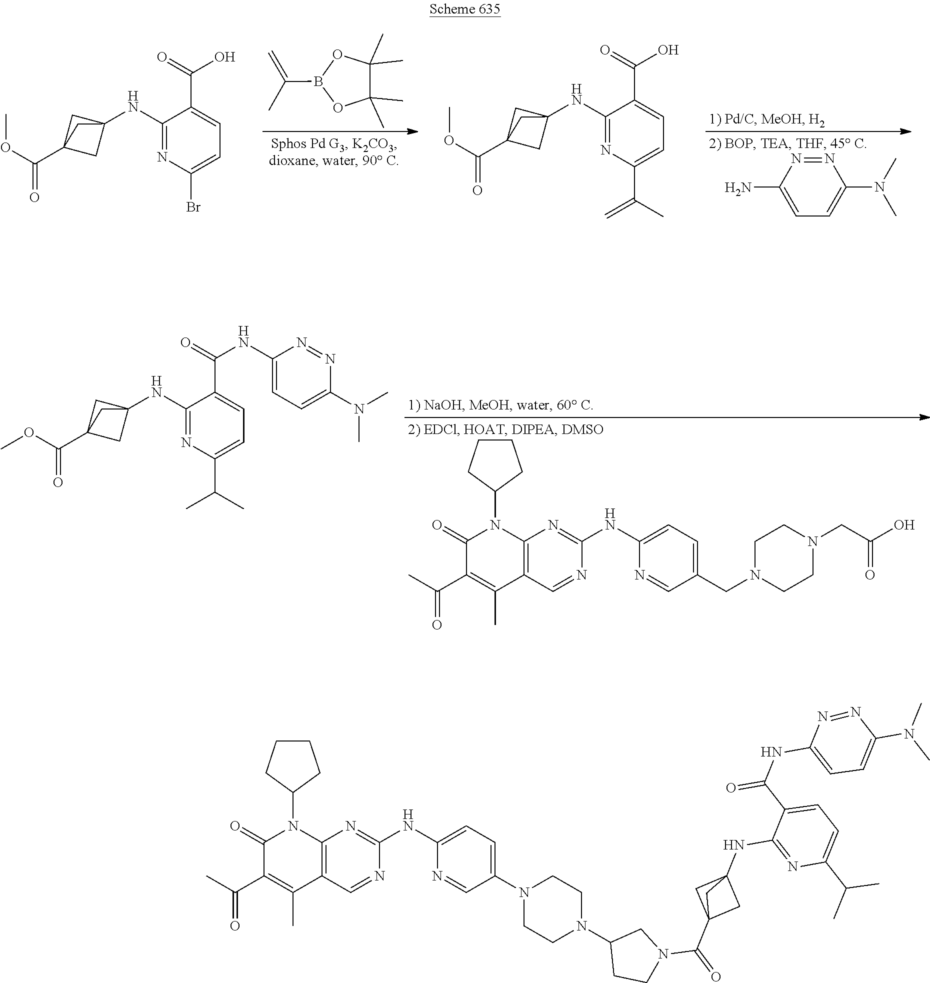

CPD-580 was synthesized following the standard procedures for preparing CPD-492 (3.5 mg, 2% yield over 5 steps) as a yellow solid. MS (ESI) m/z=909.8 [M+H]⁺.

### Example 636. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-6-cyclopropyl-N-(6-(dimethylamino)pyridazin-3-yl)nicotinamide (CPD-581)

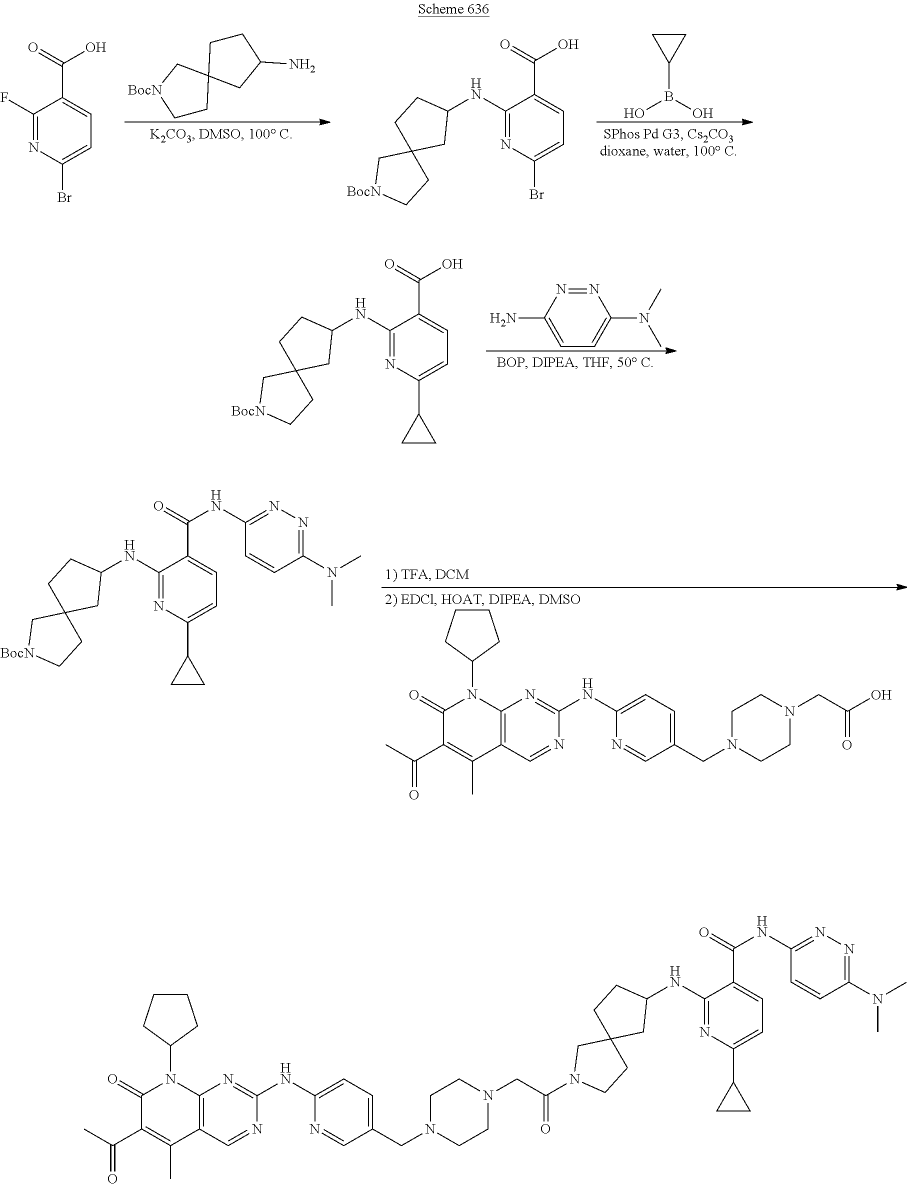

CPD-581 was synthesized following the standard procedures for preparing CPD-528 (2.7 mg, 2% yield over 5 steps) as a yellow solid. MS (ESI) m/z=924.1 [M+H]⁺.

### Example 637. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-ethylnicotinamide (CPD-582)

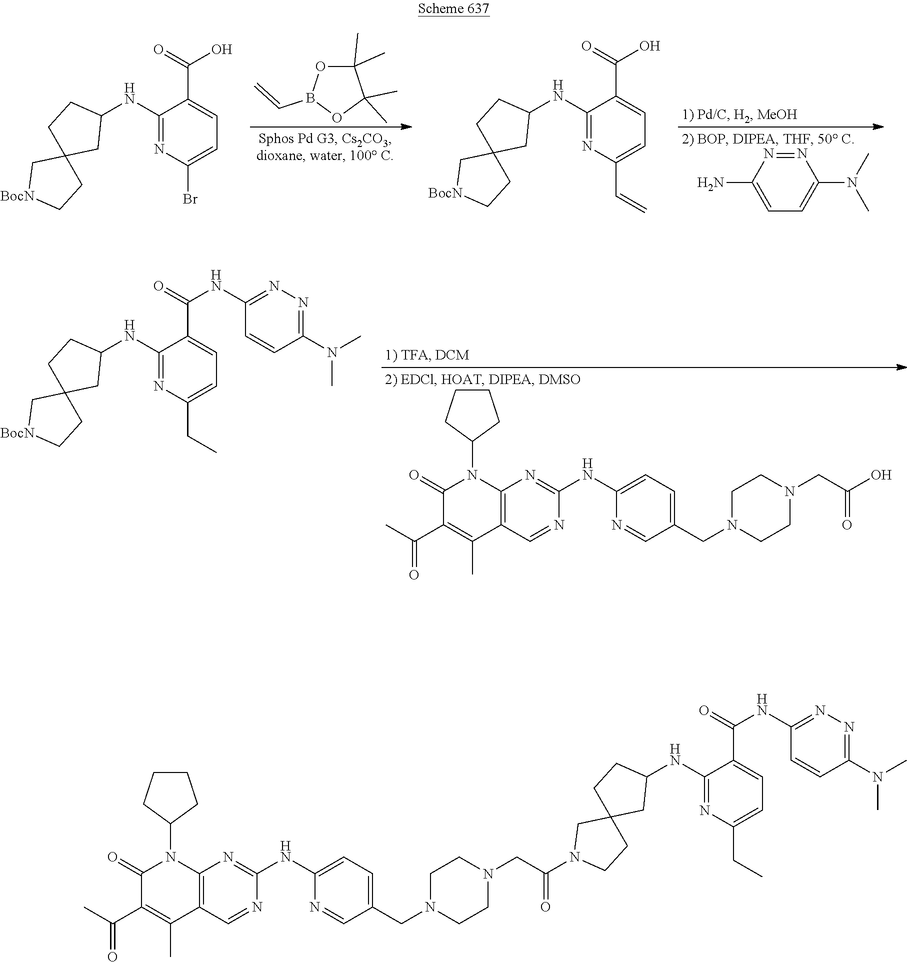

CPD-582 was synthesized following the standard procedures for preparing CPD-528 (4.5 mg, 2% yield over 5 steps) as a yellow solid. MS (ESI) m/z=912.1 [M+H]⁺.

### Example 638. 2-((2-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetyl)-2-azaspiro[4.4]nonan-7-yl)amino)-N-(6-(dimethylamino)pyridazin-3-yl)-6-isopropylnicotinamide (CPD-583)

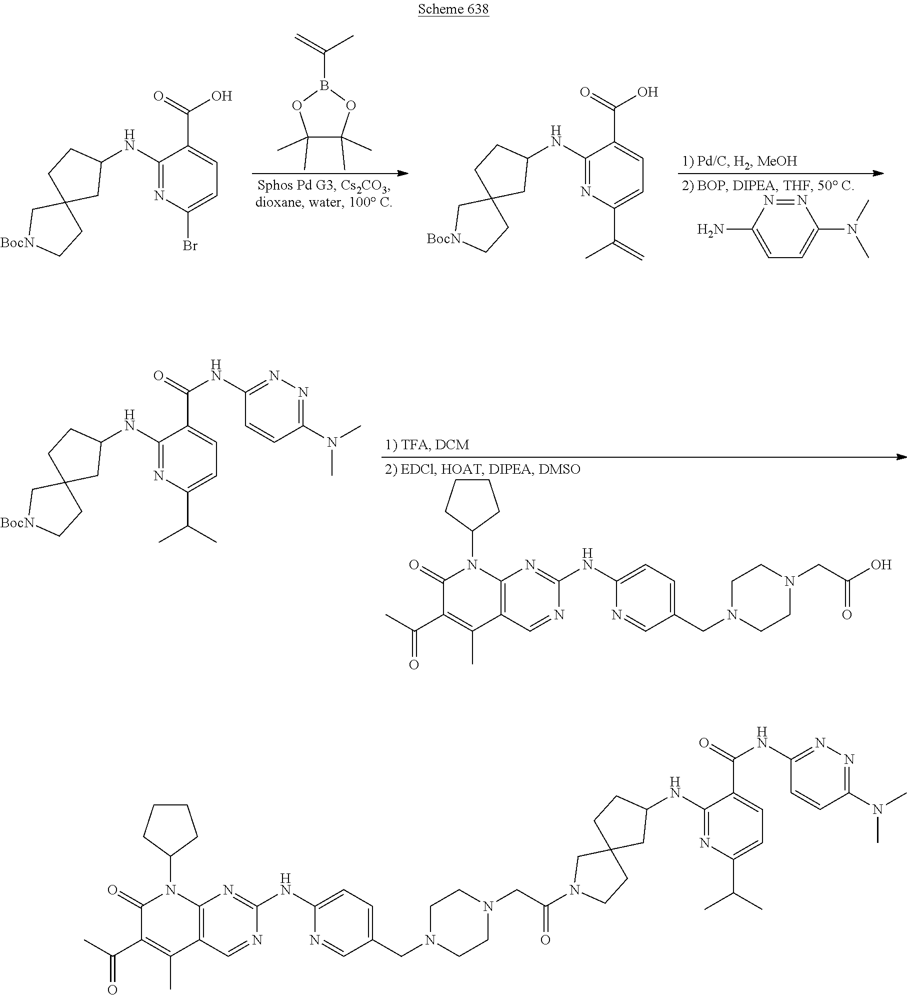

CPD-583 was synthesized following the standard procedures for preparing CPD-528 (6.1 mg, 3% yield over 5 steps) as a yellow solid. MS (ESI) m/z=925.8 [M+H]⁺.

### Example 639. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(5,6-dihydro-4H-pyrrolo[1,2-b]pyrazol-2-yl)-6-(isopropylamino) nicotinamide (CPD-584)

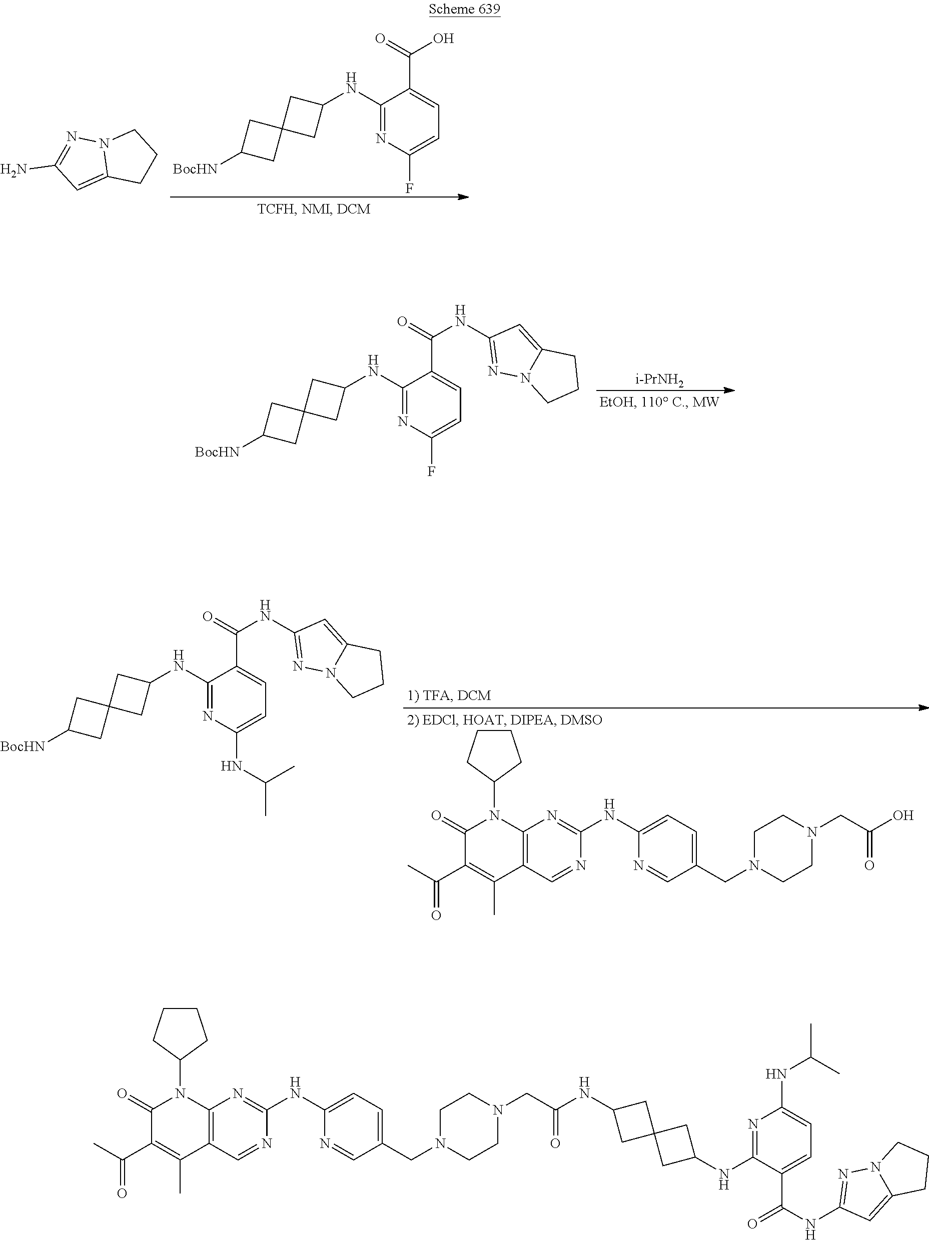

CPD-584 was synthesized following the standard procedures for preparing CPD-144 (2.6 mg, 3% yield over 4 steps) as a yellow solid. MS (ESI) m/z=912.1 [M+H]⁺.

### Example 640. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)propanamido)spiro[3.3]heptan-2-yl)amino)-N-(6-(dimethylamino)pyridazin-3-yl)-6-(isopropylamino)nicotinamide (CPD-585)

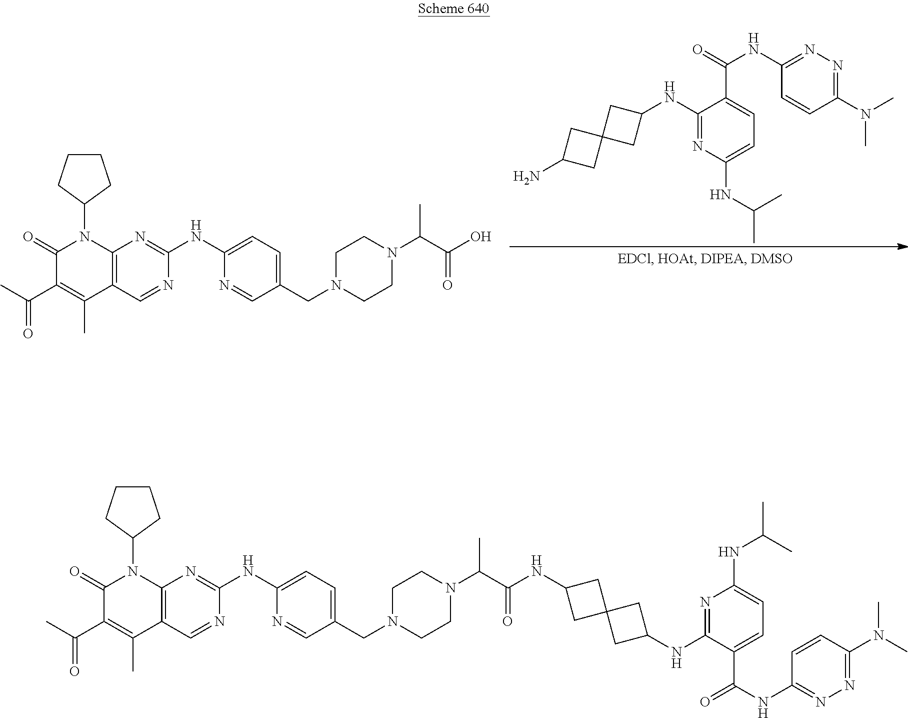 [SMILES: CC(=O)c1c(C)c2cnc(Nc3ccc(CN4CCN(C(C)C(=O)O)CC4)cn3)nc2n(C2CCCC2)c1=O ; CC(C)Nc1ccc(C(=O)Nc2ccc(N(C)C)nn2)c(NC2CC3(CC(N)C3)C2)n1 ; CC(=O)c1c(C)c2cnc(Nc3ccc(CN4CCN(C(C)C(=O)NC5CC6(C5)CC(Nc5nc(NC(C)C)ccc5C(=O)Nc5ccc(N(C)C)nn5)C6)CC4)cn3)nc2n(C2CCCC2)c1=O]

CPD-585 was synthesized following the standard procedure for preparing CPD-143 (3.6 mg, 40% yield) as a yellow solid. MS (ESI) m/z=940.9 [M+H]⁺.

### Example 641. 2-((6-(2-(4-(1-(6-((6-acetyl-8-cyclopentyl-5-methyl-7-oxo-7,8-dihydropyrido[2,3-d]pyrimidin-2-yl)amino)pyridin-3-yl)ethyl)piperazin-1-yl)acetamido)spiro[3.3]heptan-2-yl)amino)-N-(6-(dimethylamino)pyridazin-3-yl)-6-(isopropylamino)nicotinamide (CPD-586)

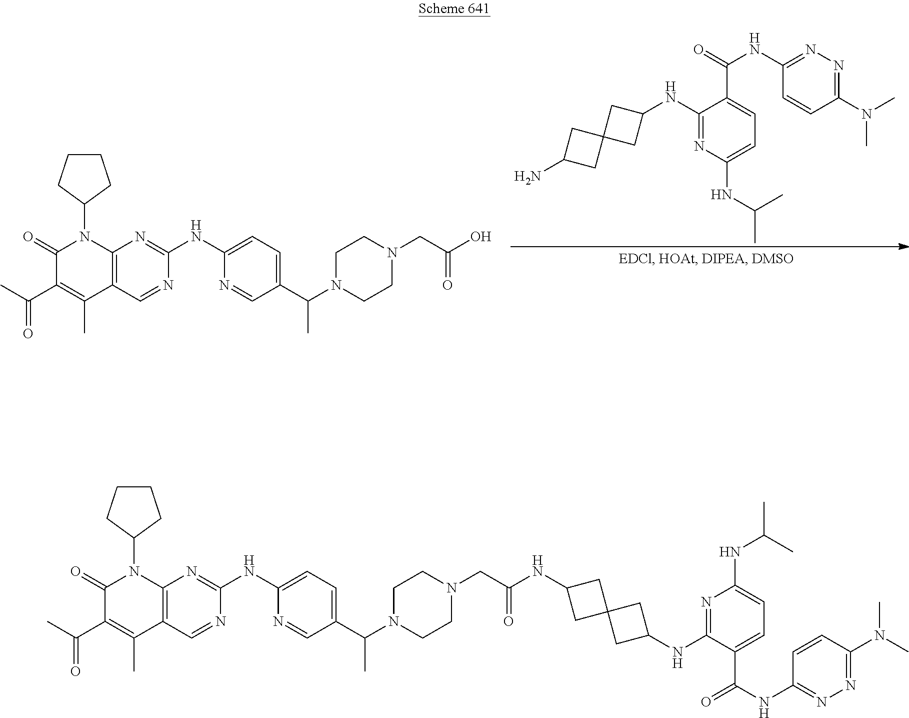 [SMILES: CC(=O)c1c(C)c2cnc(Nc3ccc(C(C)N4CCN(CC(=O)O)CC4)cn3)nc2n(C2CCCC2)c1=O ; CC(C)Nc1ccc(C(=O)Nc2ccc(N(C)C)nn2)c(NC2CC3(CC(N)C3)C2)n1 ; CC(=O)c1c(C)c2cnc(Nc3ccc(C(C)N4CCN(CC(=O)NC5CC6(C5)CC(Nc5nc(NC(C)C)ccc5C(=O)Nc5ccc(N(C)C)nn5)C6)CC4)cn3)nc2n(C2CCCC2)c1=O]

CPD-586 was synthesized following the standard procedure for preparing CPD-143 (3.2 mg, 36% yield) as a yellow solid. MS (ESI) m/z=940.9 [M+H]⁺.

### Example 642. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(1,5-dimethyl-1H-pyrazol-3-yl)-6-(isopropylamino)nicotinamide (CPD-587)

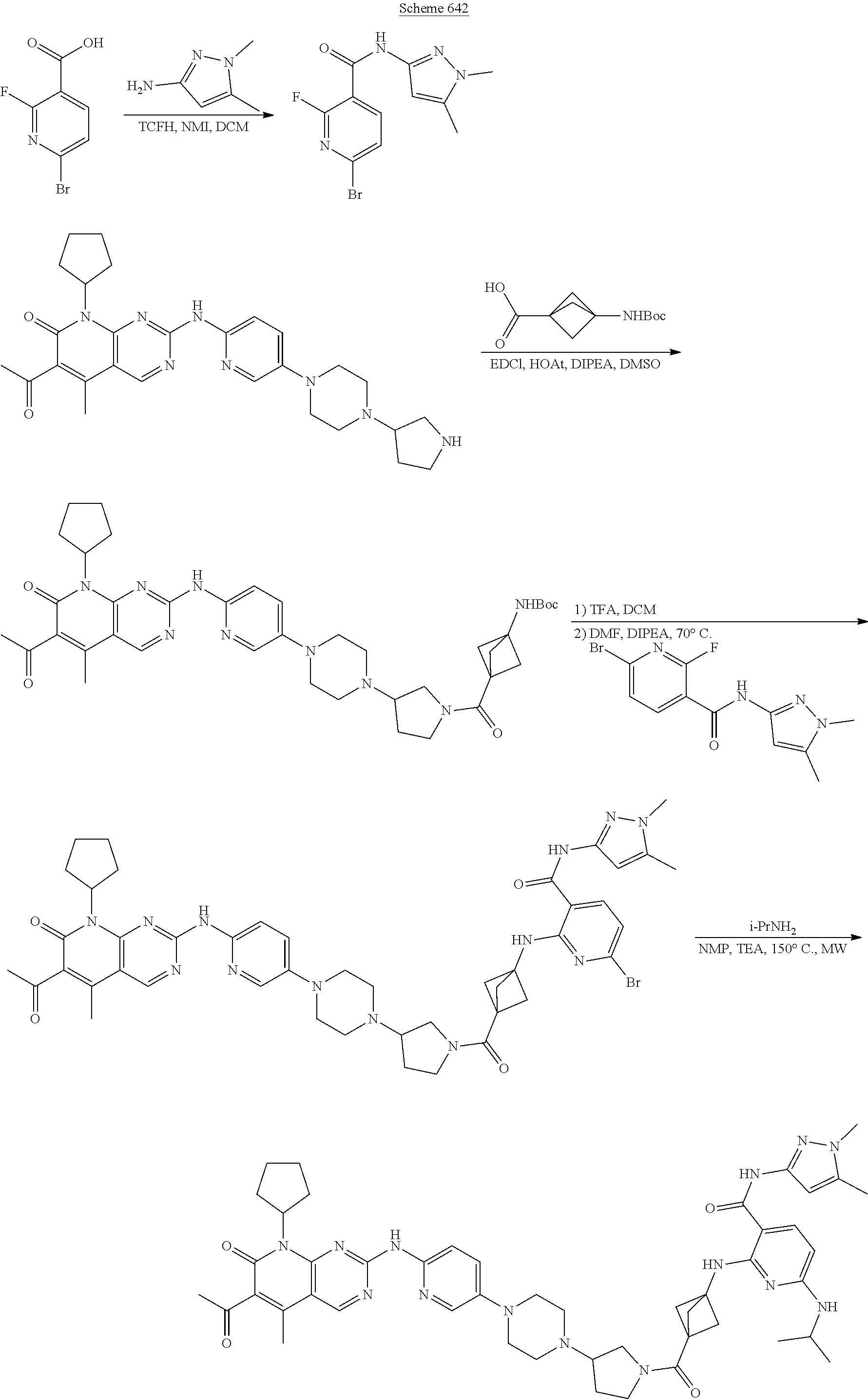

### Step 1. Synthesis of 6-bromo-N-(1,5-dimethyl-1H-pyrazol-3-yl)-2-fluoronicotinamide

To a mixture of 6-bromo-2-fluoronicotinic acid (50 mg, 0.23 mmol) and NMI (56 mg, 0.69 mmol) in dichloromethane (5 mL) were added 1,5-dimethyl-1H-pyrazol-3-amine (25 mg, 0.23 mmol) and TCFH (127.54 mg, 0.46 mmol). The mixture was stirred at rt for 3 h. The mixture was quenched with water (5 mL) and extracted with dichloromethane (3×10 mL). The combined organic phase was washed with brine, dried over Na₂SO₄, filtered and concentrated. The residue was purified by silica gel chromatography to provide the desired product (40 mg, 56% yield) as a white solid. MS (ESI) m/z=313.2 [M+H]⁺.

### Step 2. Synthesis of tert-butyl (3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)carbamate

To a mixture of 6-acetyl-8-cyclopentyl-5-methyl-2-((5-(4-(pyrrolidin-3-yl)piperazin-1-yl)pyridin-2-yl)amino)pyrido[2,3-d]pyrimidin-7(8H)-one (50 mg, 0.1 mmol), DIPEA (129.08 mg, 1.0 mmol) and HOAt (27 mg, 0.2 mmol) in DMSO (2 mL) were added EDCI (38 mg, 0.2 mmol) and 3-((tert-butoxycarbonyl)amino)bicyclo[1.1.1]pentane-1-carboxylic acid (22 mg, 0.1 mmol). The mixture was stirred at rt for 2 h. The reaction mixture was purified by prep-HPLC to provide the desired product (50 mg, 61% yield) as a light-yellow solid. MS (ESI) m/z=726.6 [M+H]⁺.

### Step 3. Synthesis of 6-acetyl-2-((5-(4-(1-(3-aminobicyclo[1.1.1]pentane-1-carbonyl)pyrrolidin-3-yl)piperazin-1-yl)pyridin-2-yl)amino)-8-cyclopentyl-5-methylpyrido[2,3-d]pyrimidin-7(8H)-one

To a solution of tert-butyl (3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)carbamate (50 mg, 0.07 mmol) in dichloromethane (5 mL) was added TFA (2 mL) at rt. The mixture was stirred at rt for 1 h. Then the solution was concentrated under reduced pressure to provide the crude product (40 mg, 78% yield) as a yellow solid. This compound was used directly in the next step without further purification. MS(ESI) m/z=626.6 [M+H]⁺.

### Step 4. Synthesis of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-6-bromo-N-(1,5-dimethyl-1H-pyrazol-3-yl)nicotinamide

To a mixture of 6-acetyl-2-((5-(4-(1-(3-aminobicyclo[1.1.1]pentane-1-carbonyl)pyrrolidin-3-yl)piperazin-1-yl)pyridin-2-yl)amino)-8-cyclopentyl-5-methylpyrido[2,3-d]pyrimidin-7(8H)-one (15 mg, 0.024 mmol) in DMF (1 mL) were added 6-bromo-N-(1,5-dimethyl-1H-pyrazol-3-yl)-2-fluoronicotinamide (7.5 mg, 0.024 mmol) and DIPEA (39 mg, 0.3 mmol). The mixture was stirred at 70 [symbol] for 5 h. After cooling down to rt, the reaction mixture was purified by prep-HPLC to provide the desired product (10 mg, 40% yield) as a yellow solid. MS (ESI) m/z=920.4[M+H]⁺.

### Step 5. Synthesis of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(1,5-dimethyl-1H-pyrazol-3-yl)-6-(isopropylamino)nicotinamide

To a solution of 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-6-bromo-N-(1,5-dimethyl-1H-pyrazol-3-yl)nicotinamide (10 mg, 0.011 mmol) in NMP (1 mL) were added TEA (33 mg, 0.33 mmol) and propan-2-amine (32 mg, 0.55 mmol). The mixture was stirred at 150 [symbol] for 1 h under microwave irradiation. After cooling down to rt, the reaction mixture was purified by prep-HPLC to provide the desired product (3 mg, 27% yield) as a yellow solid. MS (ESI) m/z=897.7 [M+H]⁺.

### Example 643. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(2-cyclopropyl-1-methyl-1H-imidazol-4-yl)-6-(isopropylamino)nicotinamide (CPD-588)

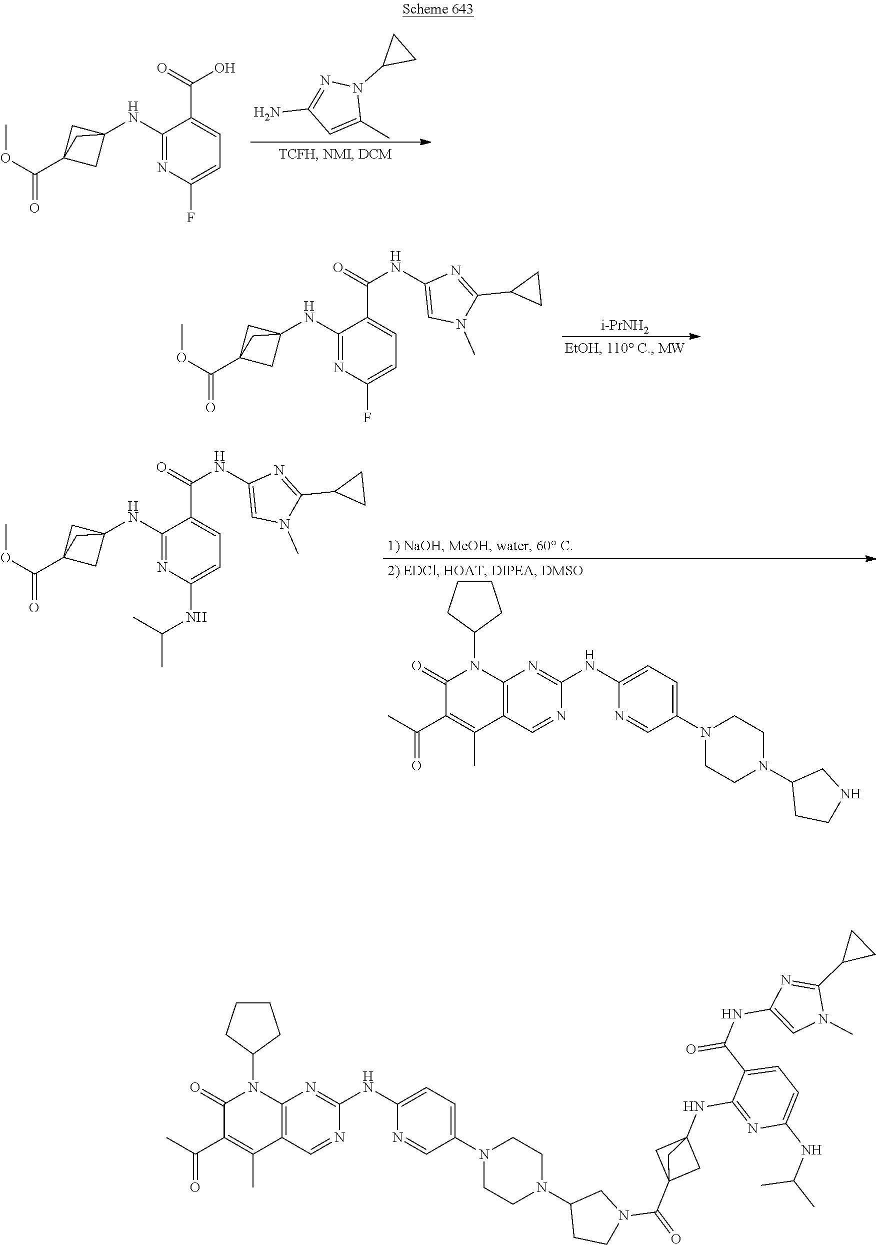

CPD-588 was synthesized following the standard procedures for preparing CPD-82 (3.8 mg, 7% yield over 4 steps) as a yellow solid. MS (ESI) m/z=923.8 [M+H]⁺.

### Example 644. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(2-cyclopropyl-1-methyl-1H-imidazol-4-yl)-6-(isopropylamino)nicotinamide (CPD-589)

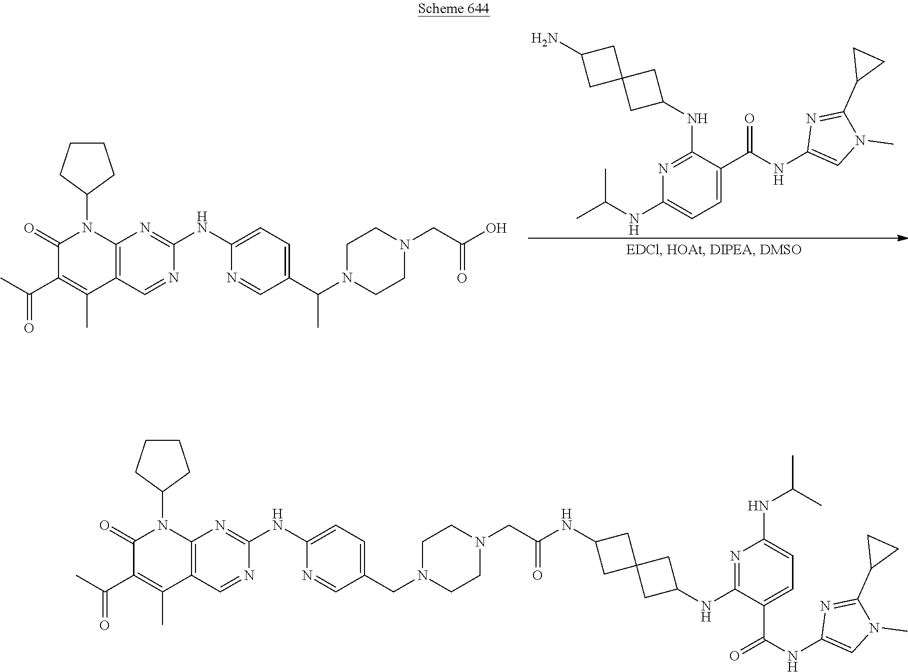 [SMILES: CC(=O)c1c(C)c2cnc(Nc3ccc(C(C)N4CCN(CC(=O)O)CC4)cn3)nc2n(C2CCCC2)c1=O ; CC(C)Nc1ccc(C(=O)Nc2cn(C)c(C3CC3)n2)c(NC2CC3(CC(N)C3)C2)n1 ; CC(=O)c1c(C)c2cnc(Nc3ccc(CN4CCN(CC(=O)NC5CC6(C5)CC(Nc5nc(NC(C)C)ccc5C(=O)Nc5cn(C)c(C7CC7)n5)C6)CC4)cn3)nc2n(C2CCCC2)c1=O]

CPD-589 was synthesized following the standard procedures for preparing CPD-143 (4.3 mg, 35% yield) as a yellow solid. MS (ESI) m/z=925.8 [M+H]⁺.

### Example 645. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(6-(dimethylamino)pyridazin-3-yl)-6-methylnicotinamide (CPD-590)

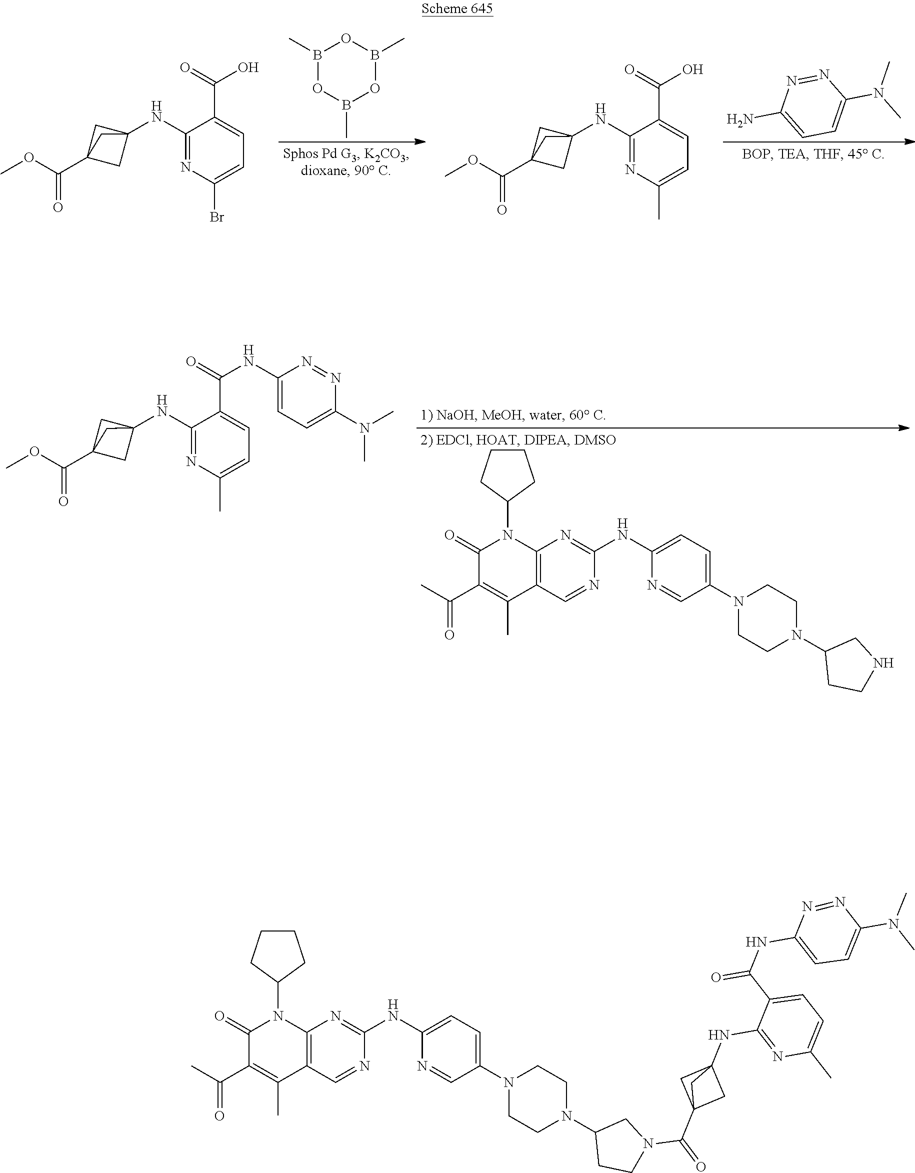

CPD-590 was synthesized following the standard procedures for preparing CPD-492 (4.0 mg, 4% yield over 4 steps) as a yellow solid. MS (ESI) m/z=881.7 [M+H]⁺.

### Example 646. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-6-(isopropylamino)-N-(5-methylisoxazol-3-yl)nicotinamide (CPD-591)

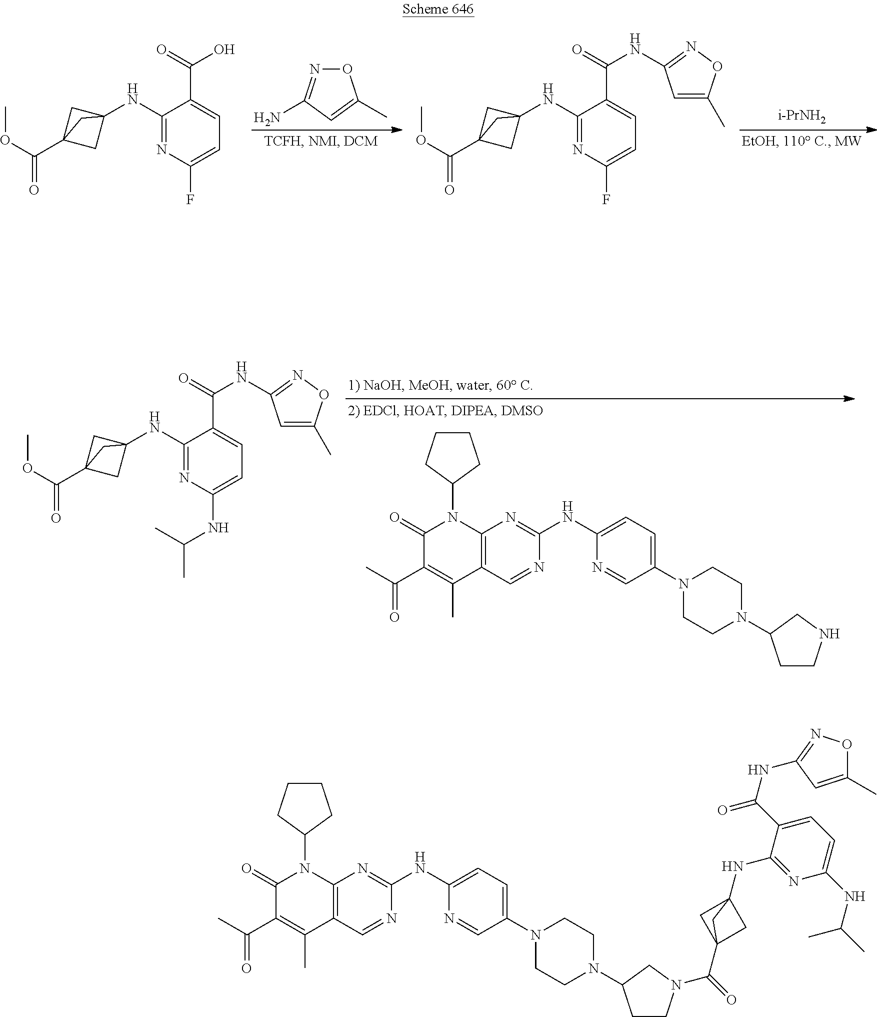

CPD-591 was synthesized following the standard procedures for preparing CPD-82 (2.6 mg, 2% yield over 4 steps) as a yellow solid. MS (ESI) m/z=884.8 [M+H]⁺.

### Example 647. 2-((3-(3-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)pyrrolidine-1-carbonyl)bicyclo[1.1.1]pentan-1-yl)amino)-N-(5,6-dihydro-4H-pyrrolo[1,2-b]pyrazol-2-yl)-6-(isopropylamino)nicotinamide (CPD-592)

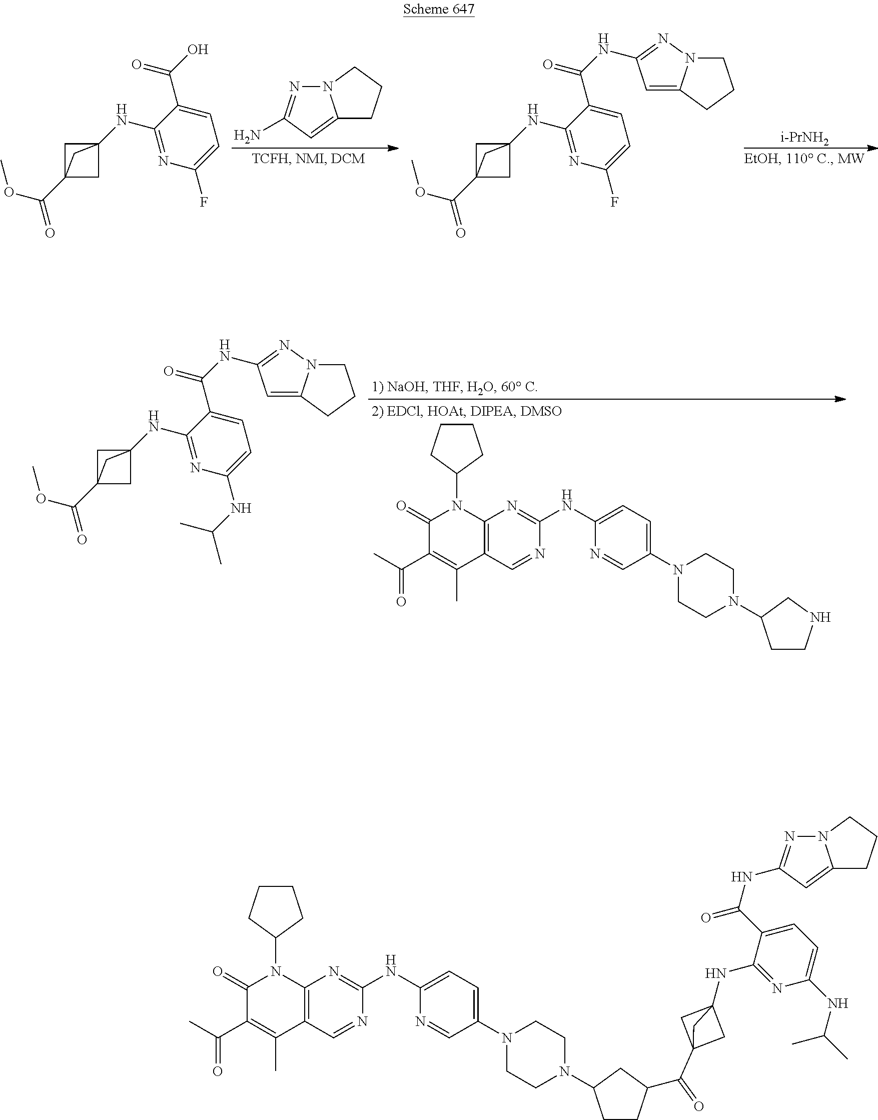

CPD-592 was synthesized following the standard procedures for preparing CPD-82 (2.3 mg, 3% yield over 4 steps) as a yellow solid. MS (ESI) m/z=909.7 [M+H]⁺.

### Example 648. 2-((6-(2-(1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)acetamido)spiro[3.3]heptan-2-yl)amino)-N-(6-(dimethylamino)pyridazin-3-yl)-6-(isopropylamino)nicotinamide (CPD-593)

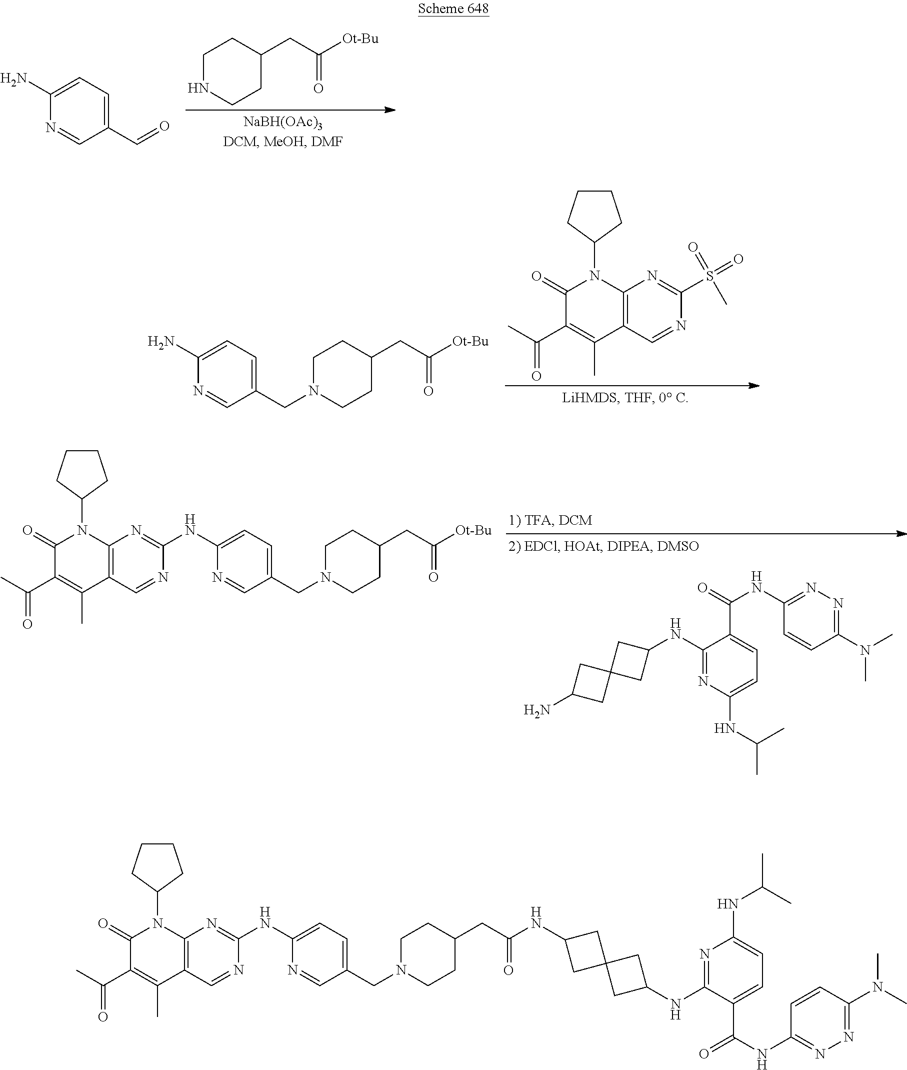

CPD-593 was synthesized following the standard procedures for preparing CPD-418 (2.9 mg, 5% yield over 4 steps) as a yellow solid. MS (ESI) m/z=925.9 [M+H]⁺.

### Example 649. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)-N-methylacetamido)spiro[3.3]heptan-2-yl)amino)-N-(6-(dimethylamino)pyridazin-3-yl)-6-(isopropylamino)nicotinamide (CPD-594)

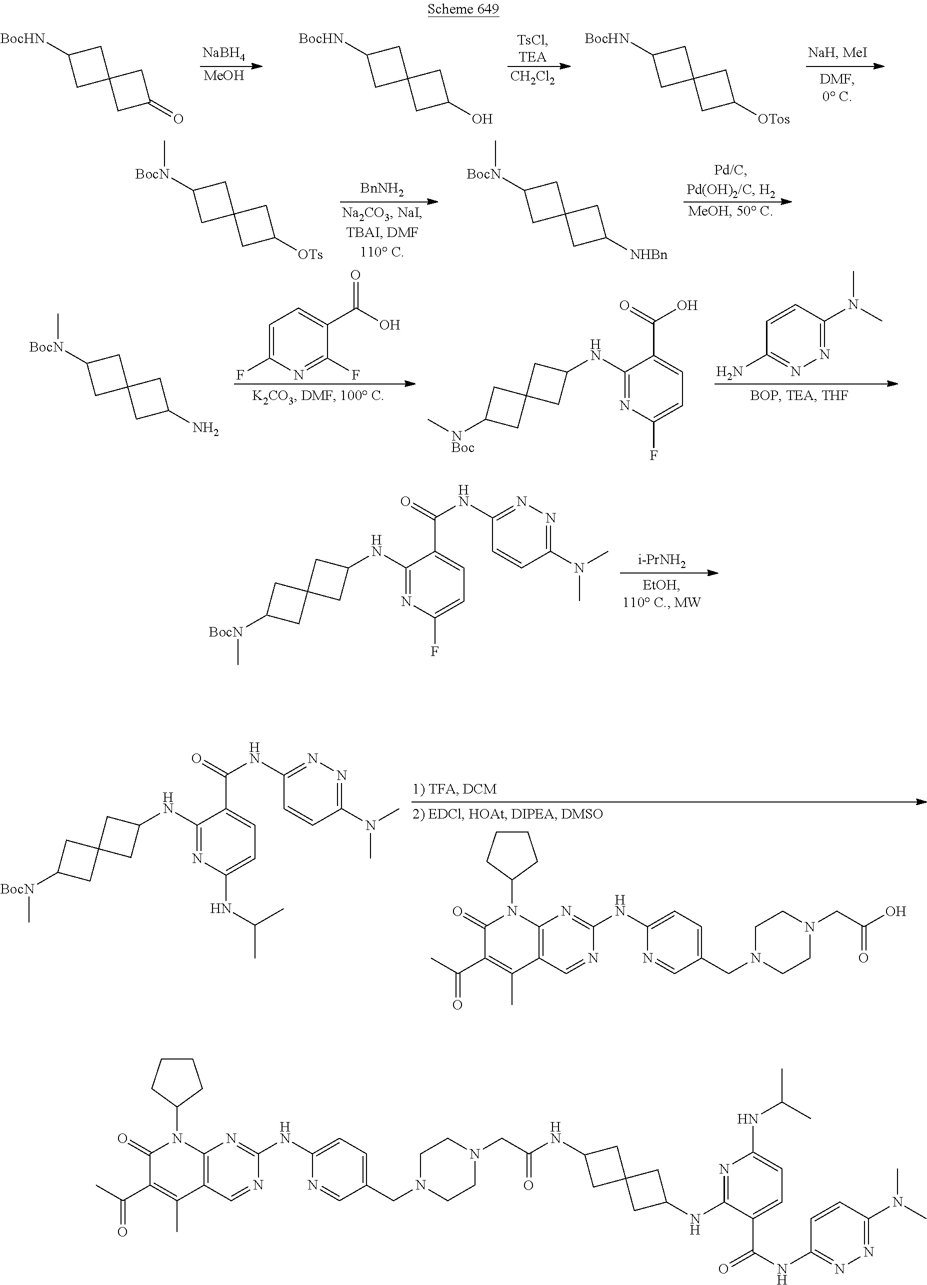

### Step 1. Synthesis of tert-butyl (6-hydroxyspiro[3.3]heptan-2-yl)carbamate

To a mixture of tert-butyl (6-oxospiro[3.3]heptan-2-yl)carbamate (400 mg, 1.8 mmol) in MeOH (6 mL) was added NaBH₄ (57 mg, 1.5 mmol) at 0° C. The mixture was stirred at rt for 2 h. The mixture was quenched with water (15 mL) and extracted with EtOAc (3×10 mL). The combined organic phase was washed with brine, dried over Na₂SO₄, filtered, and concentrated to give the crude compound (350 mg, 87% yield) as a white solid. This compound was used directly in the next step without further purification.

### Step 2. Synthesis of 6-((tert-butoxycarbonyl)amino)spiro[3.3]heptan-2-yl 4-methyl benzenesulfonate

To a solution of tert-butyl (6-hydroxyspiro[3.3]heptan-2-yl)carbamate (330 mg, 1.5 mmol) in dichloromethane (8 mL) and TEA (1 mL) was added 4-methylbenzenesulfonyl chloride (830 mg, 4.4 mmol) in portions at 0° C. The reaction mixture was stirred at rt for 24 h. The mixture was diluted with water (10 mL) and extracted with dichloromethane (3×10 mL). The combined organic phase was washed with brine, dried over Na₂SO₄, filtered and concentrated. The residue was purified by silica gel chromatography to provide the desired compound (350 mg, 63% yield) as a white solid.

### Step 3. Synthesis of 6-((tert-butoxycarbonyl)(methyl)amino)spiro[3.3]heptan-2-yl 4-methylbenzenesulfonate

A mixture of 6-((tert-butoxycarbonyl)amino)spiro[3.3]heptan-2-yl 4-methyl benzenesulfonate (350 mg, 0.9 mmol) and NaH (73 mg, 1.8 mmol, 60% in mineral oil) in DMF (4 mL) was stirred at 0° C. for 20 min, followed by adding Mel (388 mg, 2.8 mmol) at 0° C. The reaction mixture was stirred at 0° C. for 1 h. The mixture was quenched with aq. NH₄Cl solution (20 mL) and extracted with EtOAc (3×20 mL). The combined organic phase was washed with brine, dried over Na₂SO₄, filtered and concentrated. The residue was purified by silica gel chromatography to provide the desired compound (250 mg, 69% yield) as a white solid. MS (ESI) m/z=296.4 [M+H-100]⁺.

### Step 4. Synthesis of tert-butyl (6-(benzylamino)spiro[3.3]heptan-2-yl)(methyl)carbamate

A mixture of 6-((tert-butoxycarbonyl)(methyl)amino)spiro[3.3]heptan-2-yl 4-methyl benzenesulfonate (250 mg, 0.6 mmol), phenylmethanamine (203 mg, 1.9 mmol), Na₂CO₃ (335 mg, 3.2 mmol), NaI (95 mg, 0.6 mmol) and TBAI (233 mg, 0.6 mmol) in DMF (4 mL) was stirred at 100° C. for 16 h. The mixture was diluted with water (20 mL) and extracted with EtOAc (3×20 mL). The combined organic phase was washed with brine, dried over Na₂SO₄, filtered and concentrated. The residue was purified by silica gel chromatography to provide the desired compound (160 mg, 77% yield) as a white solid. MS (ESI) m/z=331.9 [M+H]⁺.

### Step 5. Synthesis of tert-butyl (6-aminospiro[3.3]heptan-2-yl)(methyl)carbamate

A mixture of tert-butyl (6-(benzylamino)spiro[3.3]heptan-2-yl)(methyl)carbamate (160 mg, 0.48 mmol), 10% Pd/C (50 mg) and 10% Pd(OH)₂/C (50 mg) in MeOH (5 mL) was degassed and stirred at 50° C. for 24 h under hydrogen balloon. The mixture was filtered and concentrated to provide the crude compound (100 mg, 86% yield) as a colorless oil. This compound was used directly in the next step without further purification.

The remaining steps were performed according to the procedures for preparing CPD-145 to provide the desired product (2.2 mg, 13% yield over 5 steps) as a yellow solid. MS (ESI) m/z=940.8 [M+H]⁺.

### Example 650. 2-((6-((1-((6-((6-acetyl-8-cyclopentyl-5-methyl-7-oxo-7,8-dihydropyrido[2,3-d]pyrimidin-2-yl)amino)pyridin-3-yl)methyl)piperidin-4-yl)(methyl)carbamoyl)spiro[3.3]heptan-2-yl)amino)-N-(6-(dimethylamino)pyridazin-3-yl)-6-(isopropylamino)nicotinamide (CPD-595)

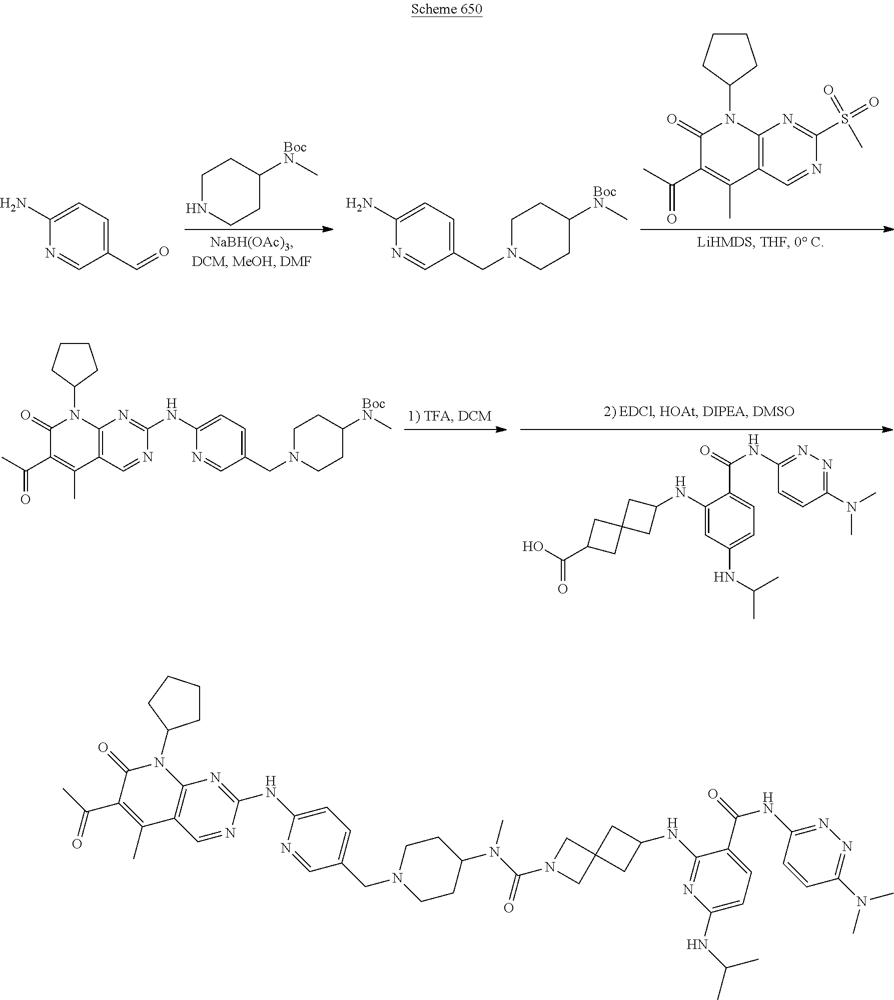

CPD-595 was synthesized following the standard procedures for preparing CPD-418 (2.9 mg, 6% yield over 4 steps) as a yellow solid. MS (ESI) m/z=925.9 [M+H]⁺.

### Example 651. 2-((6-(2-(1-((6-((6-acetyl-8-cyclopentyl-5-methyl-7-oxo-7,8-dihydropyrido[2,3-d]pyrimidin-2-yl)amino)pyridin-3-yl)methyl)pyrrolidin-3-yl)acetamido)spiro[3.3]heptan-2-yl)amino)-N-(6-(dimethylamino)pyridazin-3-yl)-6-(isopropylamino)nicotinamide (CPD-596)

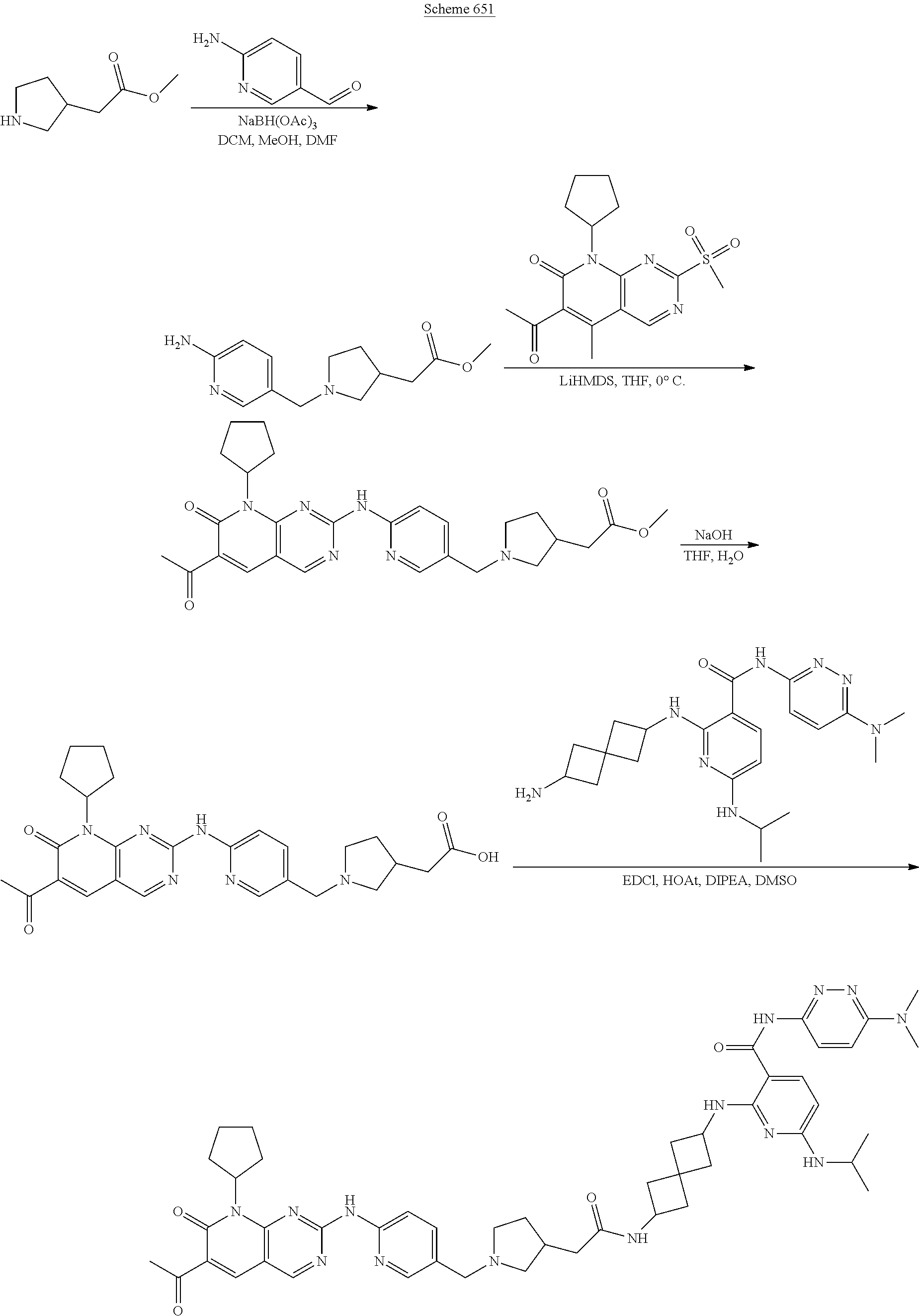

CPD-596 was synthesized following the standard procedures for preparing CPD-488 (2.5 mg, 2% yield over 4 steps) as a yellow solid. MS (ESI) m/z=911.9 [M+H]⁺.

### Example 652. 2-((trans-4-(3-(4-(4-((6-acetyl-8-cyclopentyl-5-methyl-7-oxo-7,8-dihydropyrido[2,3-d]pyrimidin-2-yl)amino)cyclohexyl)piperazin-1-yl)pyrrolidine-1-carbonyl)cyclohexyl)amino)-N-(6-(dimethylamino)pyridazin-3-yl)-6-ethylnicotinamide (CPD-597)

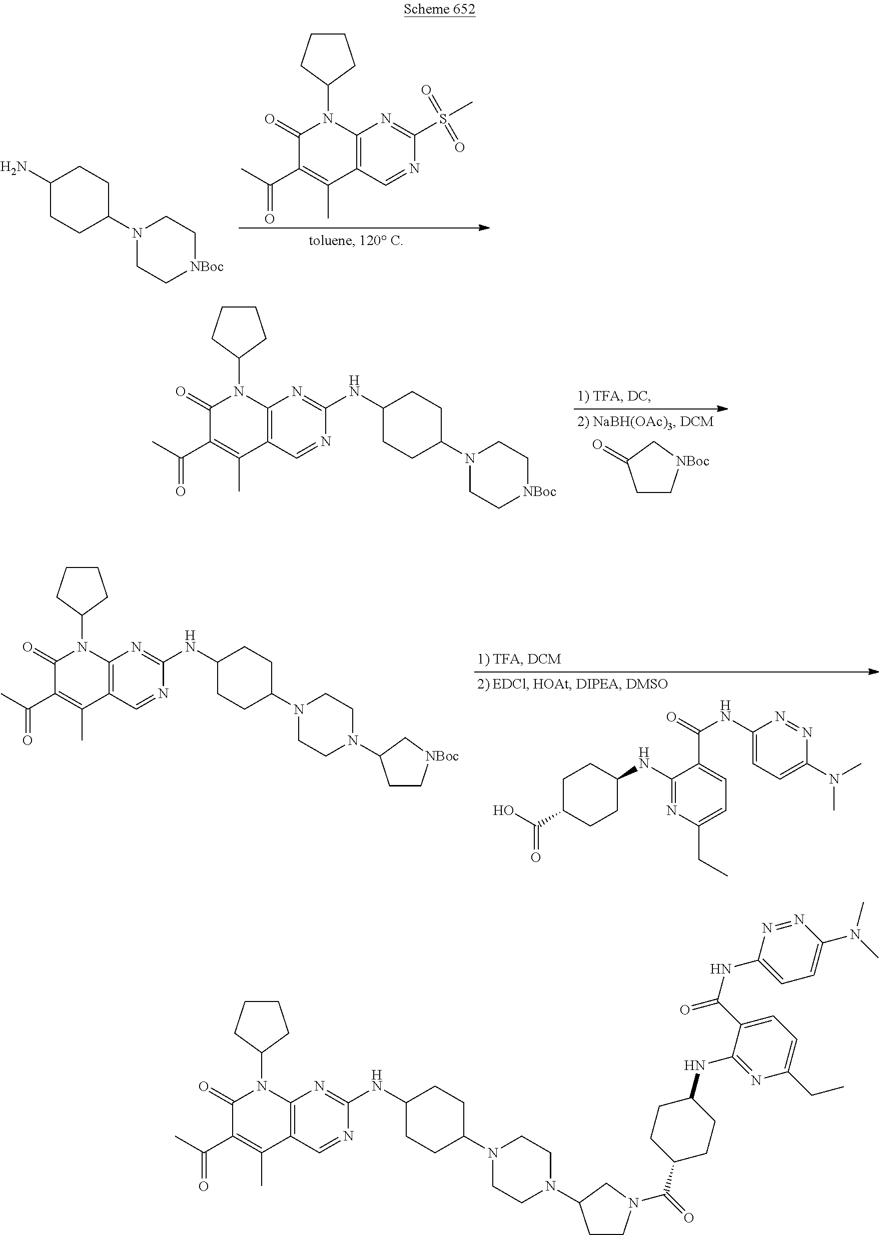

### Step 1. Synthesis of tert-butyl 4-(4-((6-acetyl-8-cyclopentyl-5-methyl-7-oxo-7,8-dihydropyrido[2,3-d]pyrimidin-2-yl)amino)cyclohexyl)piperazine-1-carboxylate

To a solution of tert-butyl 4-(4-aminocyclohexyl)piperazine-1-carboxylate (100 mg, 343.19 μmol) in toluene (10 mL) was added 6-acetyl-8-cyclopentyl-5-methyl-2-(methylsulfonyl)pyrido[2,3-d]pyrimidin-7(8H)-one (119.91 mg, 343.19 μmol) at rt. The mixture was stirred at 120° C. for 2 h. The reaction mixture was cooled to rt and concentrated. The resulting residue was purified by silica gel chromatography to give the desired product (120 mg, 62% yield) as a red solid. MS (ESI) m/z=533.8 [M+H]⁺.

### Step 2. Synthesis of 6-acetyl-8-cyclopentyl-5-methyl-2-((4-(piperazin-1-yl)cyclohexyl)amino)pyrido[2,3-d]pyrimidin-7(8H)-one

To a solution of tert-butyl 4-(4-((6-acetyl-8-cyclopentyl-5-methyl-7-oxo-7,8-dihydropyrido[2,3-d]pyrimidin-2-yl)amino)cyclohexyl)piperazine-1-carboxylate (120 mg, 214.02 μmol) in dichloromethane (4 mL) was added TFA (2 mL). The mixture was stirred at rt for 1 h. The reaction mixture was concentrated to give the crude product (70 mg, 710% yield) as a red oil, which was used directly in the next step without further purification. MS (ESI) m/z 461.5 [M+H]⁺.

The remaining steps were performed according to the procedures for preparing CPD-510 to provide the desired product (9.0 mg, 10% yield over 3 steps) as a yellow solid. MS (ESI) m/z=917.2 [M+H]⁺.

### Example 653. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-4-ethylbenzamide (CPD-598)

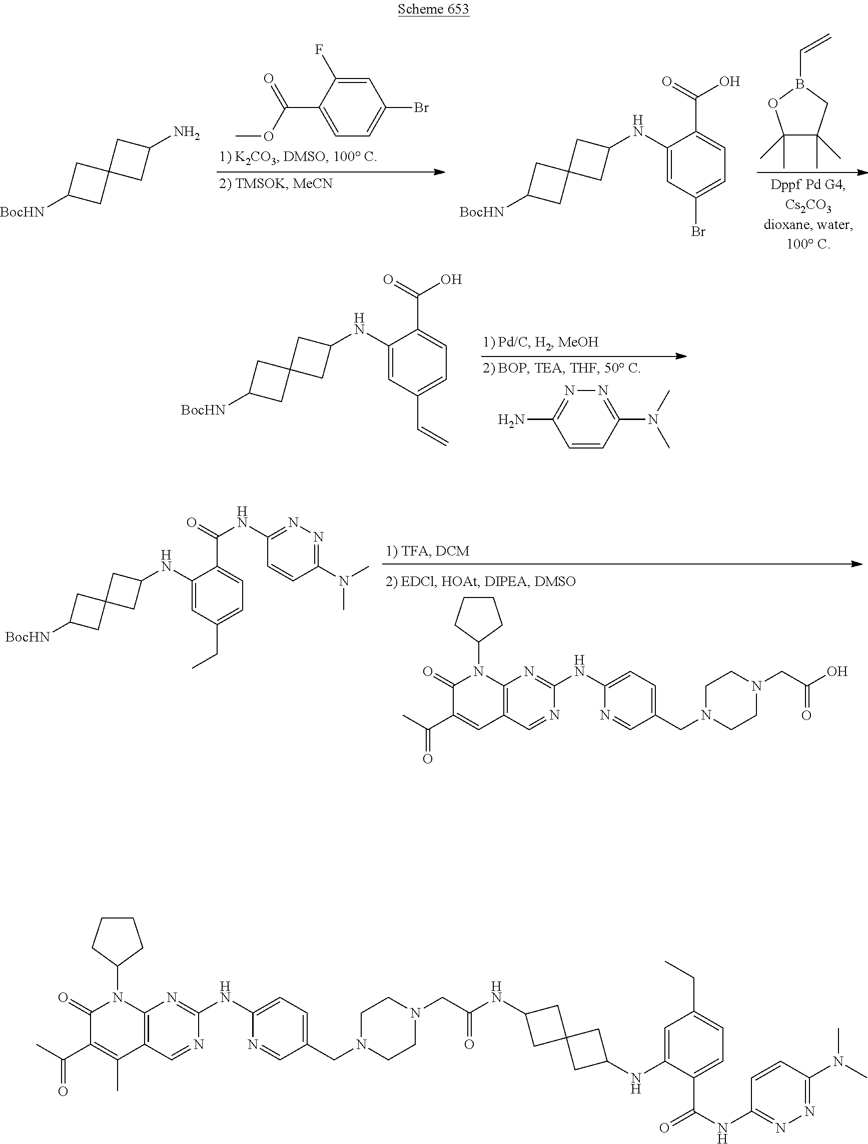

### Step 1. Synthesis of methyl 4-bromo-2-((6-((tert-butoxycarbonyl)amino)spiro[3.3]heptan-2-yl)amino)benzoate

To a solution of tert-butyl (6-aminospiro[3.3]heptan-2-yl)carbamate (700 mg, 3.09 mmol) in DMSO (10 mL) were added methyl 4-bromo-2-fluorobenzoate (720.78 mg, 3.09 mmol) and K₂CO₃ (427.48 mg, 3.09 mmol) at rt. The mixture was stirred at 100° C. for 16 h. The reaction mixture was cooled to rt and filtered. The filtrate was purified by reverse-phase chromatography to give the desired product (500 mg, 37% yield) as a pale-white solid. MS (ESI) m/z=383.2 [M+H-56]⁺.

### Step 2. Synthesis of 4-bromo-2-((6-((tert-butoxycarbonyl)amino)spiro[3.3]heptan-2-yl)amino)benzoic acid

To a solution of methyl 4-bromo-2-((6-((tert-butoxycarbonyl)amino)spiro[3.3]heptan-2-yl)amino)benzoate (500 mg, 1.14 mmol) in MeCN (10 mL) was added TMSOK (1.46 g, 11.38 mmol) at rt. The mixture was stirred at rt for 16 h. The reaction mixture was quenched with water (100 mL), acidified with aq. HCl (1 M), and extracted with dichloromethane (3×50 mL). The combined organic layers were washed with saturated brine (50 mL), dried over anhydrous sodium sulfate, filtered, and concentrated. The resulting residue was purified by reverse-phase chromatography to give the desired product (400 mg, 83% yield) as a pale-white solid. MS (ESI) m/z=369.2 [M+H-56]⁺.

### Step 3. Synthesis of 2-((6-((tert-butoxycarbonyl)amino)spiro[3.3]heptan-2-yl)amino)-4-vinylbenzoic acid

To a solution of 4-bromo-2-((6-((tert-butoxycarbonyl)amino)spiro[3.3]heptan-2-yl)amino)benzoic acid (100 mg, 235.12 μmol) in dioxane (5 mL) and water (1 mL) were added 4,4,5,5-tetramethyl-2-vinyl-1,3,2-dioxaborolane (36.21 mg, 235.12 μmol), DppfPd G4 (10.93 mg, 11.76 mol) and Cs₂CO₃ (76.61 mg, 235.12 mol) at rt. The mixture was stirred at 100° C. under inert atmosphere for 16 h. The reaction mixture was cooled to rt, filtered and concentrated. The resulting residue was purified by reverse-phase chromatography to give the desired product (40 mg, 46% yield) as a pale-white solid. MS (ESI) m/z=373.3 [M+H]⁺.

### Step 4. Synthesis of 2-((6-((tert-butoxycarbonyl)amino)spiro[3.3]heptan-2-yl)amino)-4-ethylbenzoic acid

To a solution of 2-((6-((tert-butoxycarbonyl)amino)spiro[3.3]heptan-2-yl)amino)-4-vinylbenzoic acid (40 mg, 107.39 μmol) in MeOH (4 mL) was added 5% Pd/C (5 mg). The mixture was stirred at rt under hydrogen atmosphere for 3 h. The reaction mixture was filtered and concentrated. The resulting residue was purified by silica gel chromatography to give the desired product (27 mg, 67% yield). MS (ESI) m/z=375.4 [M+H]⁺.

### Step 5. Synthesis of tert-butyl (6-((2-((6-(dimethylamino)pyridazin-3-yl)carbamoyl)-5-ethylphenyl)amino)spiro[3.3]heptan-2-yl)carbamate

To a solution of 2-((6-((tert-butoxycarbonyl)amino)spiro[3.3]heptan-2-yl)amino)-4-ethylbenzoic acid (27 mg, 72.10 μmol) in THF (4 mL) were added N³,N³-dimethylpyridazine-3,6-diamine (9.96 mg, 72.10 μmol), BOP (47.83 mg, 108.15 μmol) and TEA (36.48 mg, 360.51 μmol). The mixture was stirred at 50° C. for 16 h. The reaction mixture was cooled to rt and concentrated. The resulting residue was purified by silica gel chromatography to give the desired product (3 mg, 8% yield) as a pale-white solid. MS (ESI) m/z=495.6 [M+H]⁺.

### Step 6. Synthesis of 2-((6-aminospiro[3.3]heptan-2-yl)amino)-N-(6-(dimethylamino)pyridazin-3-yl)-4-ethylbenzamide

A solution of tert-butyl (6-((2-((6-(dimethylamino)pyridazin-3-yl)carbamoyl)-5-ethylphenyl)amino)spiro[3.3]heptan-2-yl)carbamate (3 mg, 6.07 μmol) in dichloromethane (2 mL) and TFA (1 mL) was stirred at rt for 1 h. The reaction mixture was concentrated to give the crude product (2 mg, 65% yield) as a yellow oil, which was used directly in the next step without further purification. MS(ESI) m/z=395.4 [M+H]⁺.

### Step 7. Synthesis of 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetamido)spiro[3.3]heptan-2-yl)amino)-N-(6-(dimethylamino)pyridazin-3-yl)-4-ethylbenzamide

To a mixture of 2-((6-aminospiro[3.3]heptan-2-yl)amino)-N-(6-(dimethylamino)pyridazin-3-yl)-4-ethylbenzamide (2 mg, 5.07 μmol), EDCI (1.46 mg, 7.60 μmol) and HOAt (1.04 mg, 7.60 μmol) in DMSO (1 mL) were added DIPEA (3.28 mg, 25.35 μmol) and 2-(4-((6-((6-acetyl-8-cyclopentyl-5-methyl-7-oxo-7,8-dihydropyrido[2,3-d]pyrimidin-2-yl)amino)pyridin-3-yl)methyl)piperazin-1-yl)acetic acid (2.63 mg, 5.07 mol). The mixture was stirred at rt for 16 h, before it was purified by prep-HPLC to provide the desired product (0.8 mg, 16% yield) as a yellow solid. MS (ESI) m/z=897.1 [M+H]⁺.

### Example 654. 2-((6-(2-(4-((6-((6-acetyl-8-cyclopentyl-5-methyl-7-oxo-7,8-dihydropyrido[2,3-d]pyrimidin-2-yl)amino)pyridin-3-yl)methyl)piperidin-1-yl)acetamido)spiro[3.3]heptan-2-yl)amino)-N-(6-(dimethylamino)pyridazin-3-yl)-6-(isopropylamino)nicotinamide (CPD-599)

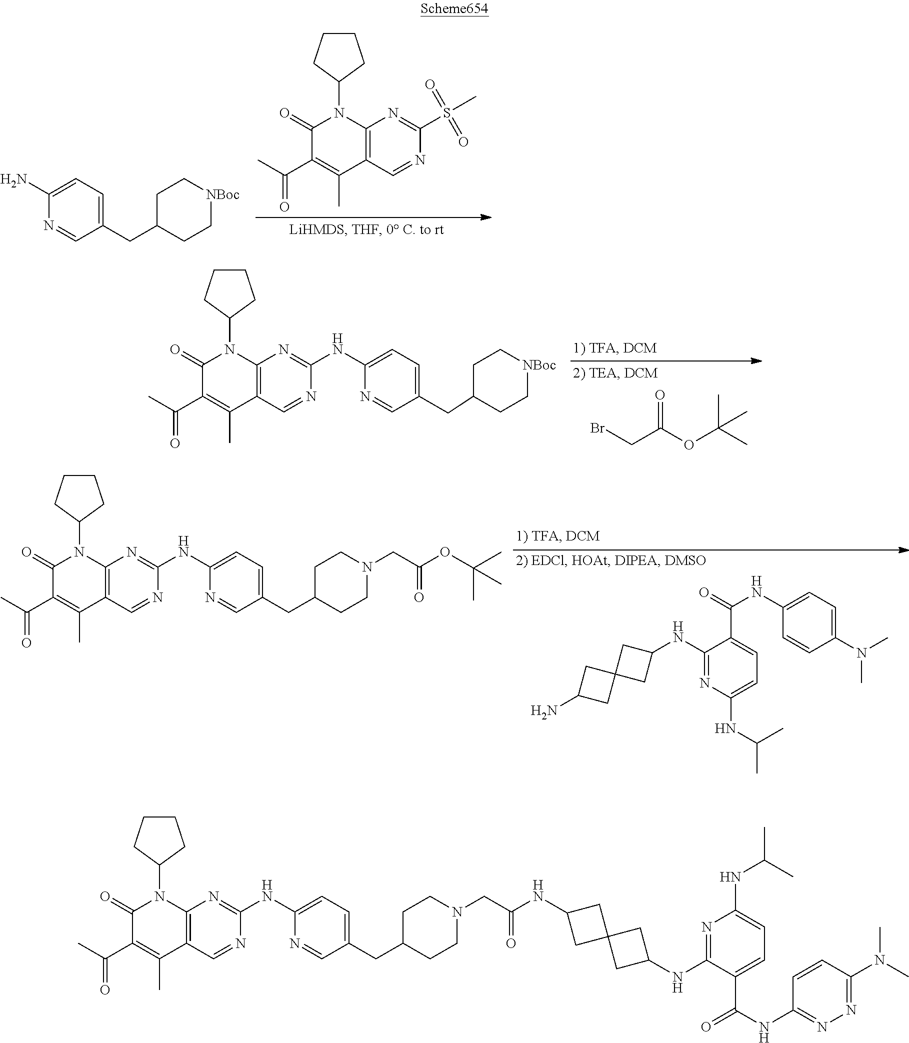

CPD-599 was synthesized following the standard procedures for preparing CPD-373 (4.2 mg, 1% yield over 5 steps) as a yellow solid. MS (ESI) m/z=925.5 [M+H]⁺.

### Example 655. N-(6-(dimethylamino)pyridazin-3-yl)-2-((3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropyl)amino)-6-(methylamino)nicotinamide (CPD-600)

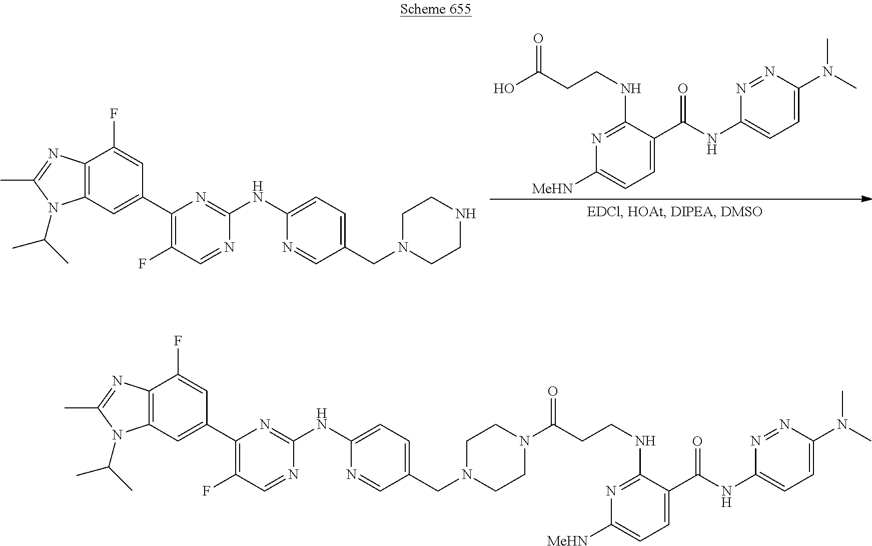

CPD-600 was synthesized following the standard procedure for preparing CPD-143 (2.5 mg, 32% yield) as a yellow solid. MS (ESI) m/z=820.7 [M+H]⁺.

### Example 656. N-(6-(dimethylamino)pyridazin-3-yl)-2-((4-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-4-oxobutyl)amino)-6-(methylamino)nicotinamide (CPD-601)

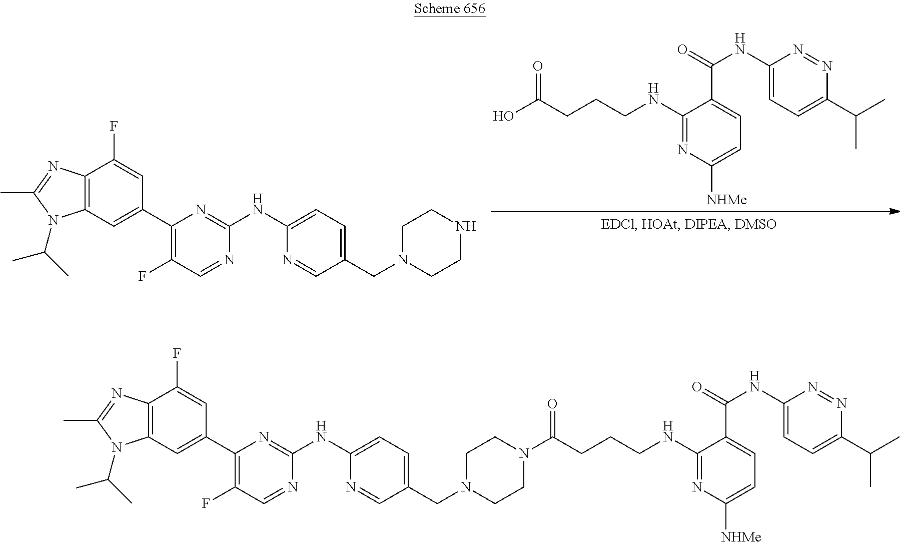 [SMILES: Cc1nc2c(F)cc(-c3nc(Nc4ccc(CN5CCNCC5)cn4)ncc3F)cc2n1C(C)C ; CNc1ccc(C(=O)Nc2ccc(C(C)C)nn2)c(NCCCC(=O)O)n1 ; CNc1ccc(C(=O)Nc2ccc(C(C)C)nn2)c(NCCCC(=O)N2CCN(Cc3ccc(Nc4ncc(F)c(-c5cc(F)c6nc(C)n(C(C)C)c6c5)n4)nc3)CC2)n1]

CPD-601 was synthesized following the standard procedure for preparing CPD-143 (2.4 mg, 30% yield) as a yellow solid. MS (ESI) m/z=834.7 [M+H]⁺.

### Example 657. N-(6-(dimethylamino)pyridazin-3-yl)-2-((5-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-5-oxopentyl)amino)-6-(methylamino)nicotinamide (CPD-602)

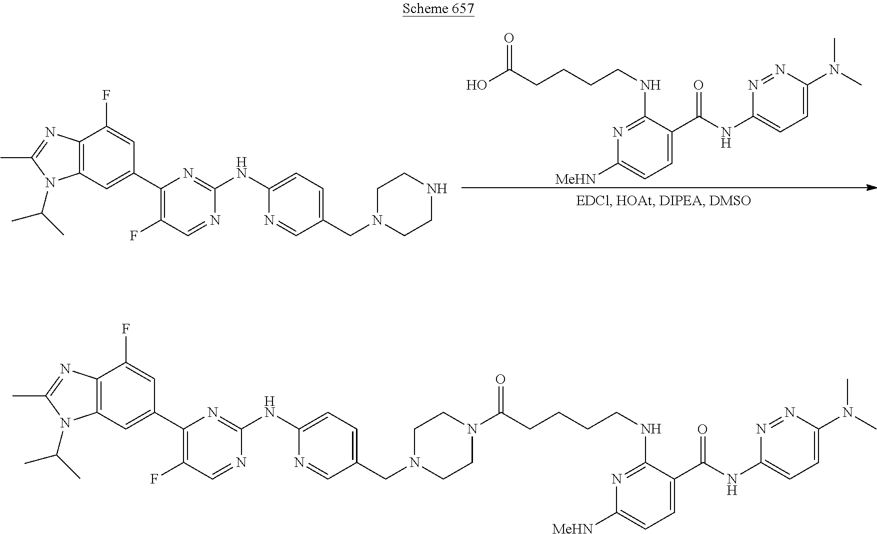

CPD-602 was synthesized following the standard procedure for preparing CPD-143 (2.3 mg, 29% yield) as a yellow solid. MS (ESI) m/z=848.7 [M+H]⁺.

### Example 658. N-(6-(dimethylamino)pyridazin-3-yl)-2-((6-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-6-oxohexyl)amino)-6-(methylamino)nicotinamide (CPD-603)

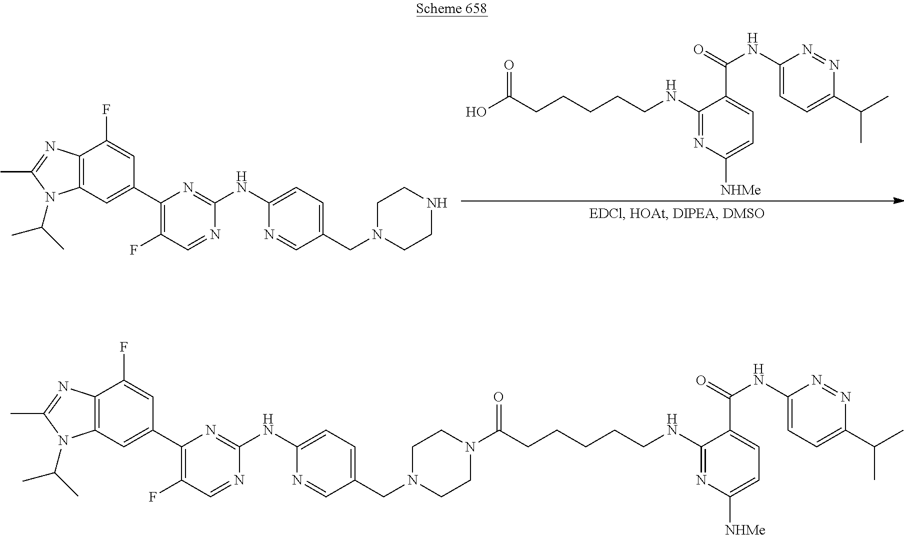 [SMILES: Cc1nc2c(F)cc(-c3nc(Nc4ccc(CN5CCNCC5)cn4)ncc3F)cc2n1C(C)C ; CNc1ccc(C(=O)Nc2ccc(C(C)C)nn2)c(NCCCCCC(=O)O)n1 ; CNc1ccc(C(=O)Nc2ccc(C(C)C)nn2)c(NCCCCCC(=O)N2CCN(Cc3ccc(Nc4ncc(F)c(-c5cc(F)c6nc(C)n(C(C)C)c6c5)n4)nc3)CC2)n1]

CPD-603 was synthesized following the standard procedure for preparing CPD-143 (2.3 mg, 28% yield) as a yellow solid. MS (ESI) m/z=862.7 [M+H]⁺.

### Example 659. N-(6-(dimethylamino)pyridazin-3-yl)-2-((7-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-7-oxoheptyl)amino)-6-(methylamino)nicotinamide (CPD-604)

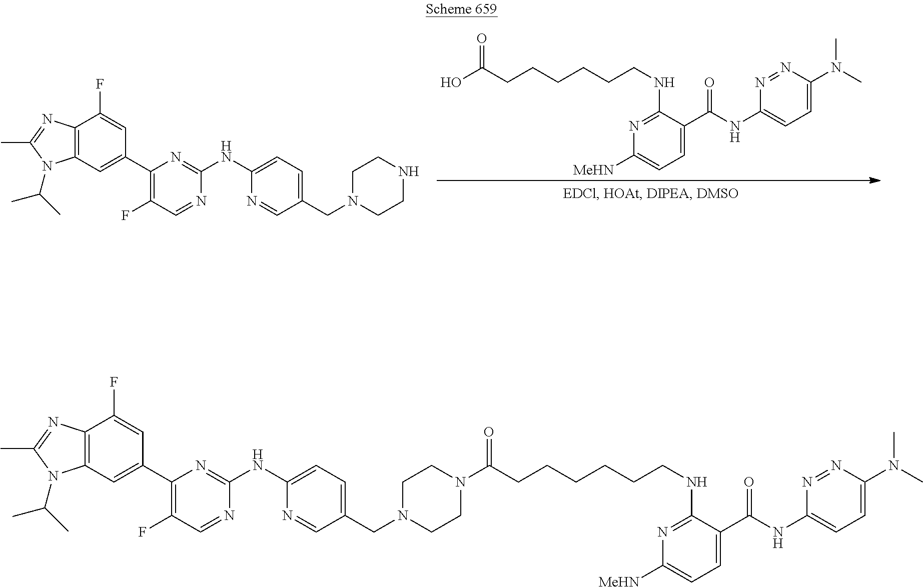

CPD-604 was synthesized following the standard procedure for preparing CPD-143 (3.0 mg, 36% yield) as a yellow solid. MS (ESI) m/z=876.7 [M+H]⁺.

### Example 660. N-(6-(dimethylamino)pyridazin-3-yl)-2-((8-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-8-oxooctyl)amino)-6-(methylamino)nicotinamide (CPD-605)

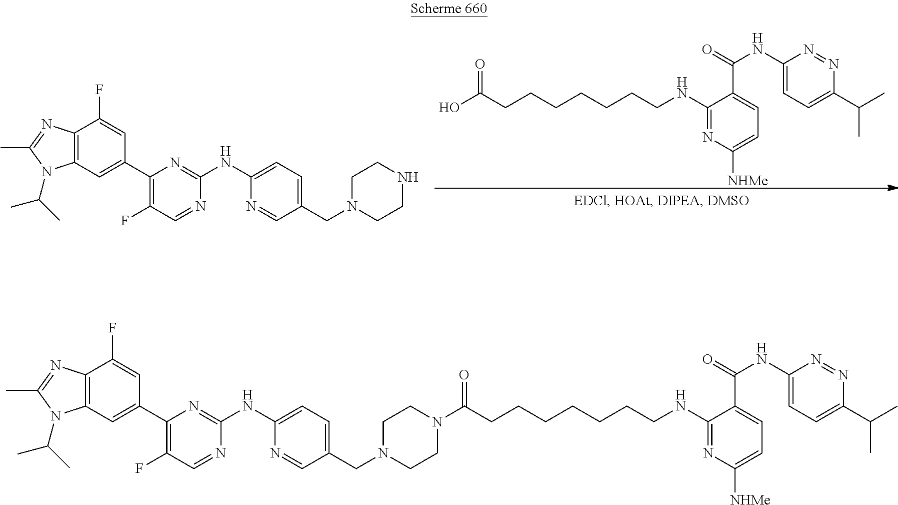 [SMILES: Cc1nc2c(F)cc(-c3nc(Nc4ccc(CN5CCNCC5)cn4)ncc3F)cc2n1C(C)C ; CNc1ccc(C(=O)Nc2ccc(C(C)C)nn2)c(NCCCCCCCC(=O)O)n1 ; CNc1ccc(C(=O)Nc2ccc(C(C)C)nn2)c(NCCCCCCCC(=O)N2CCN(Cc3ccc(Nc4ncc(F)c(-c5cc(F)c6nc(C)n(C(C)C)c6c5)n4)nc3)CC2)n1]

CPD-605 was synthesized following the standard procedure for preparing CPD-143 (2.3 mg, 27% yield) as a yellow solid. MS (ESI) m/z=890.7 [M+H]⁺.

### Example 661. N-(6-(dimethylamino)pyridazin-3-yl)-2-((2-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropoxy)ethyl)amino)-6-(methylamino)nicotinamide (CPD-606)

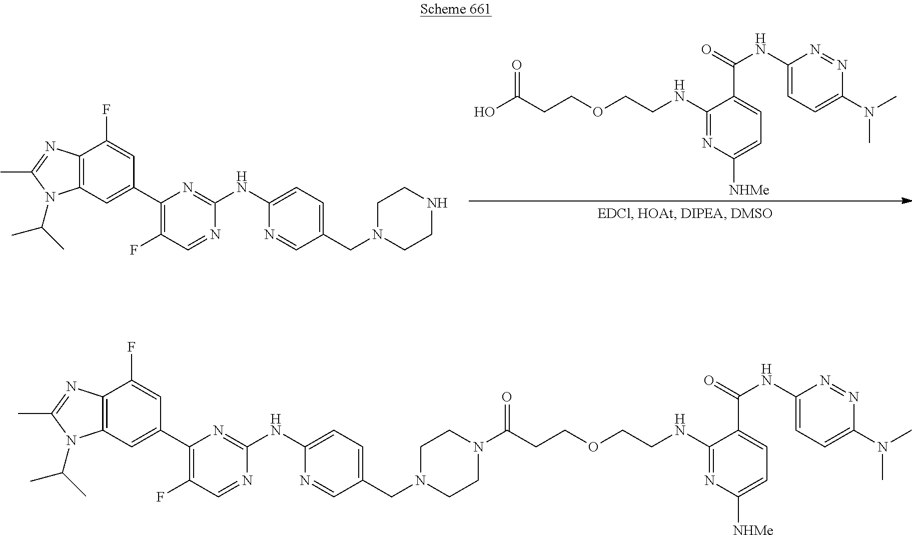

CPD-606 was synthesized following the standard procedure for preparing CPD-143 (2.2 mg, 27% yield) as a yellow solid. MS (ESI) m/z=864.7 [M+H]⁺.

### Example 662. N-(6-(dimethylamino)pyridazin-3-yl)-2-((2-(2-(3-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-3-oxopropoxy)ethoxy)ethyl)amino)-6-(methylamino)nicotinamide (CPD-607)

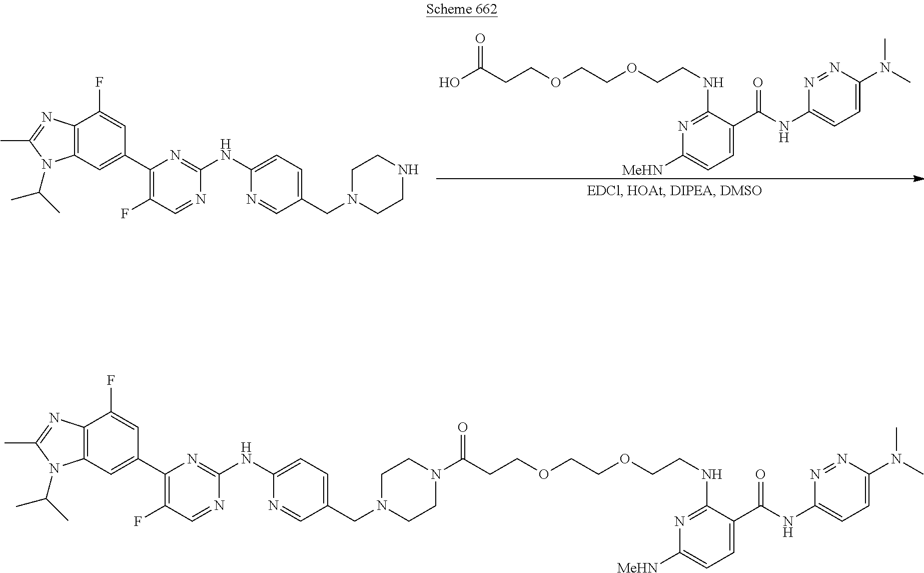

CPD-607 was synthesized following the standard procedure for preparing CPD-143 (2.6 mg, 34% yield) as a yellow solid. MS (ESI) nm/z=908.7 [M+H]⁺.

### Example 663. N-(6-(dimethylamino)pyridazin-3-yl)-2-((2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethyl)amino)-6-(methylamino)nicotinamide (CPD-608)

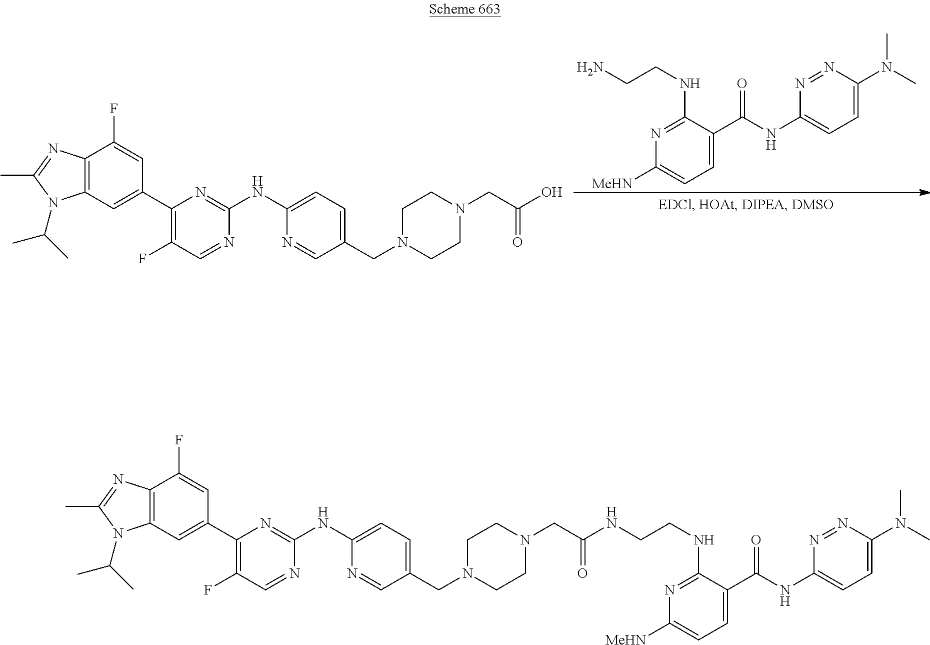

CPD-608 was synthesized following the standard procedure for preparing CPD-143 (2.7 mg, 21% yield) as a yellow solid. MS (ESI) m/z=849.7 [M+H]⁺.

### Example 664. N-(6-(dimethylamino)pyridazin-3-yl)-2-((2-(2-(4-((6-((5-fluoro-4-(4-fluoro-1-isopropyl-2-methyl-1H-benzo[d1]imidazol-6-yl)pyrimidin-2-yl)amino)pyridin-3-yl)methyl)piperazin-1-yl)acetamido)ethyl)amino)-6-(methylamino)nicotinamide (CPD-609)

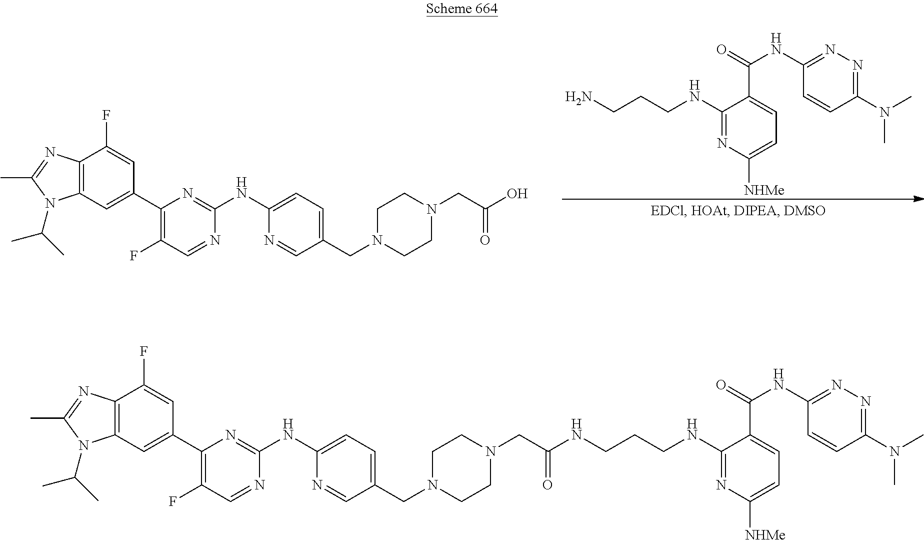

CPD-609 was synthesized following the standard procedure for preparing CPD-143 (2.1 mg, 29% yield) as a yellow solid. MS (ESI) m/z=863.7 [M+H]⁺.

### Example 665. N-(6-(dimethylamino)pyridazin-3-yl)-2-((4-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)butyl)amino)-6-(methylamino)nicotinamide (CPD-610)

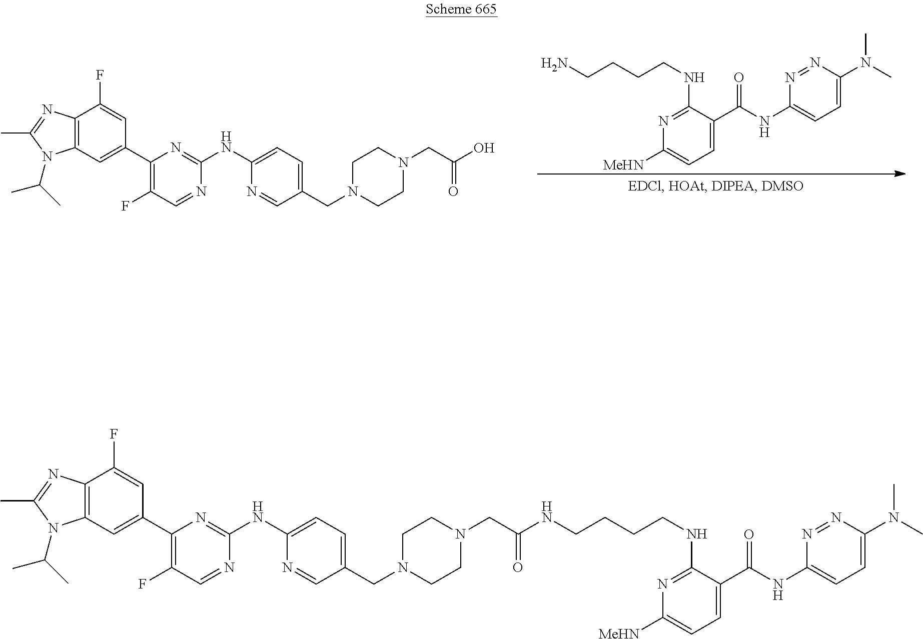

CPD-610 was synthesized following the standard procedure for preparing CPD-143 (2.6 mg, 39% yield) as a yellow solid. MS (ESI) m/z=877.7 [M+H]⁺.

### Example 666. N-(6-(dimethylamino)pyridazin-3-yl)-2-((5-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)pentyl)amino)-6-(methylamino)nicotinamide (CPD-611)

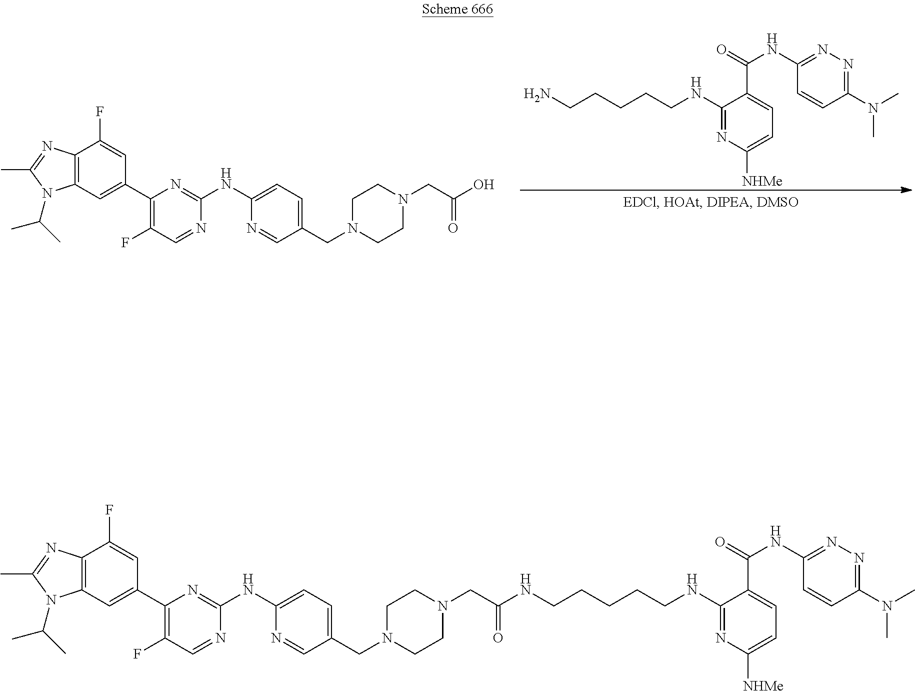

CPD-611 was synthesized following the standard procedure for preparing CPD-143 (2.6 mg, 35% yield) as a yellow solid. MS (ESI) m/z=891.7 [M+H]⁺.

### Example 667. N-(6-(dimethylamino)pyridazin-3-yl)-2-((6-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)hexyl)amino)-6-(methylamino)nicotinamide (CPD-612)

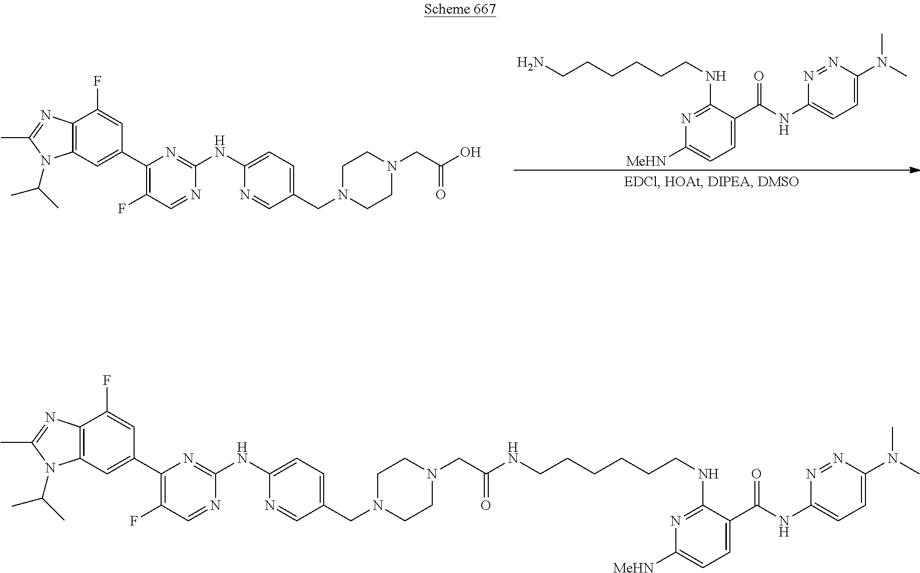

CPD-612 was synthesized following the standard procedure for preparing CPD-143 (2.7 mg, 35% yield) as a yellow solid. MS (ESI) m/z=905.7 [M+H]⁺.

### Example 668. N-(6-(dimethylamino)pyridazin-3-yl)-2-((7-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)heptyl)amino)-6-(methylamino)nicotinamide (CPD-613)

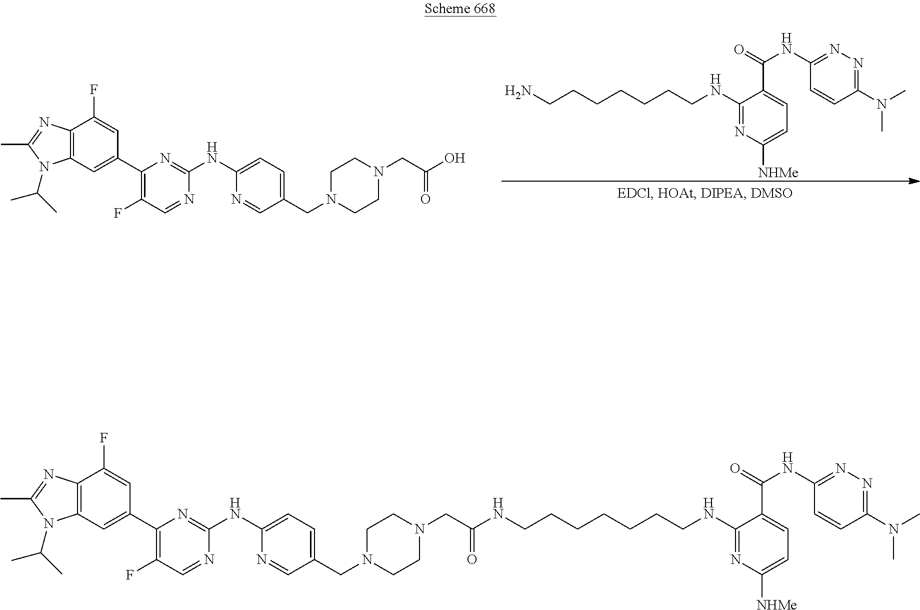

CPD-613 was synthesized following the standard procedure for preparing CPD-143 (2.4 mg, 31% yield) as a yellow solid. MS (ESI) m/z=919.8 [M+H]⁺.

### Example 669. N-(6-(dimethylamino)pyridazin-3-yl)-2-((8-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)octyl)amino)-6-(methylamino)nicotinamide (CPD-614)

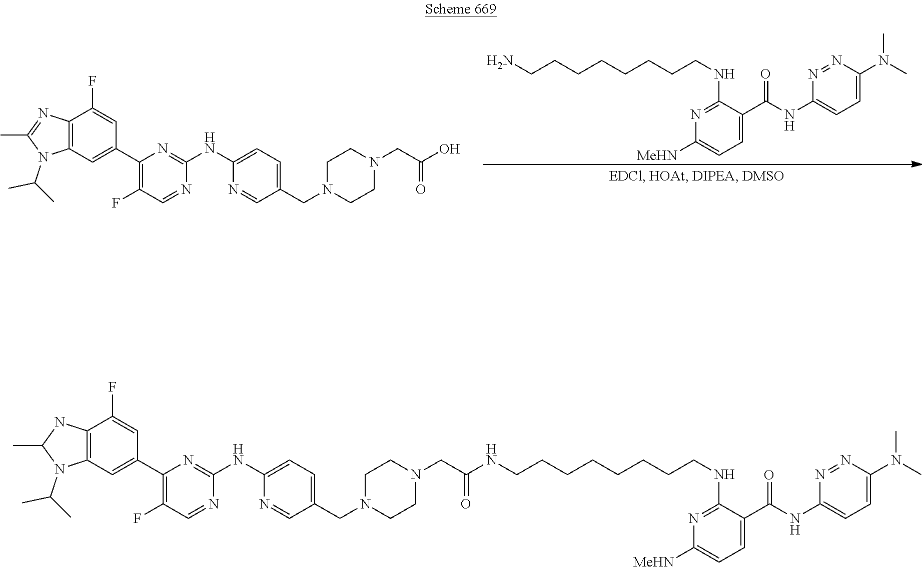

CPD-614 was synthesized following the standard procedure for preparing CPD-143 (1.3 mg, 16% yield) as a yellow solid. MS (ESI) m/z=933.8 [M+H]⁺.

### Example 670. N-(6-(dimethylamino)pyridazin-3-yl)-2-((2-(2-(2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)acetamido)ethoxy)ethyl)amino)-6-(methylamino)nicotinamide (CPD-615)

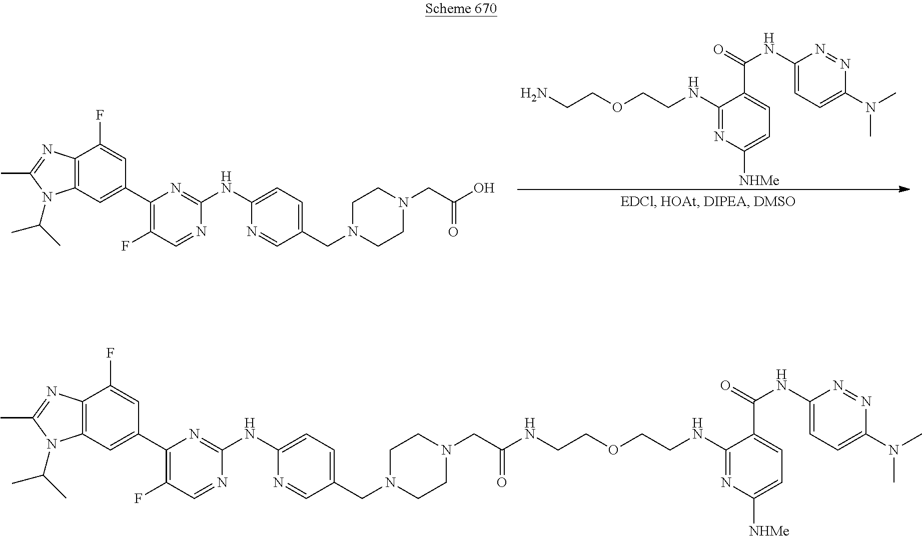

CPD-615 was synthesized following the standard procedure for preparing CPD-143 (2.3 mg, 35% yield) as a yellow solid. MS (ESI) m/z=893.7 [M+H]⁺.

### Example 671. 7-cyclopentyl-2-((5-(4-(3-((3-((6-(dimethylamino)pyridazin-3-yl)carbamoyl)-6-(methylamino)pyridin-2-yl)amino)propanoyl)piperazin-1-yl)pyridin-2-yl)amino)-N,N-dimethyl-7H-pyrrolo[2,3-d]pyrimidine-6-carboxamide (CPD-616)

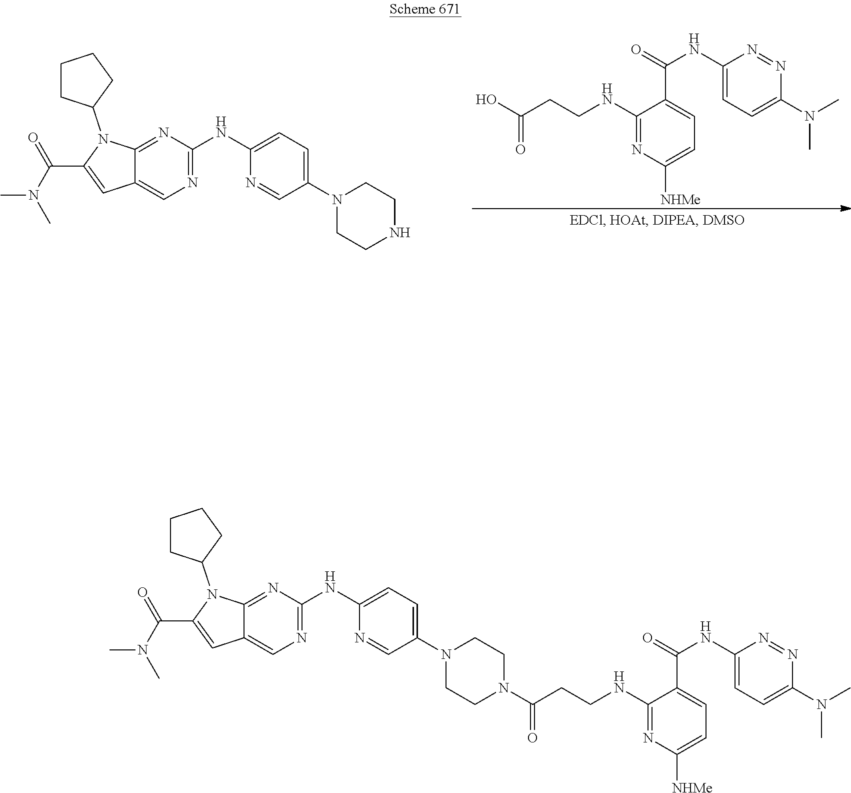

CPD-616 was synthesized following the standard procedure for preparing CPD-143 (2.5 mg, 31% yield) as a yellow solid. MS (ESI) m/z=776.6 [M+H]⁺.

### Example 672. 7-cyclopentyl-2-((5-(4-(4-((3-((6-(dimethylamino)pyridazin-3-yl)carbamoyl)-6-(methylamino)pyridin-2-yl)amino)butanoyl)piperazin-1-yl)pyridin-2-yl)amino)-N,N-dimethyl-7H-pyrrolo[2,3-d]pyrimidine-6-carboxamide (CPD-617)

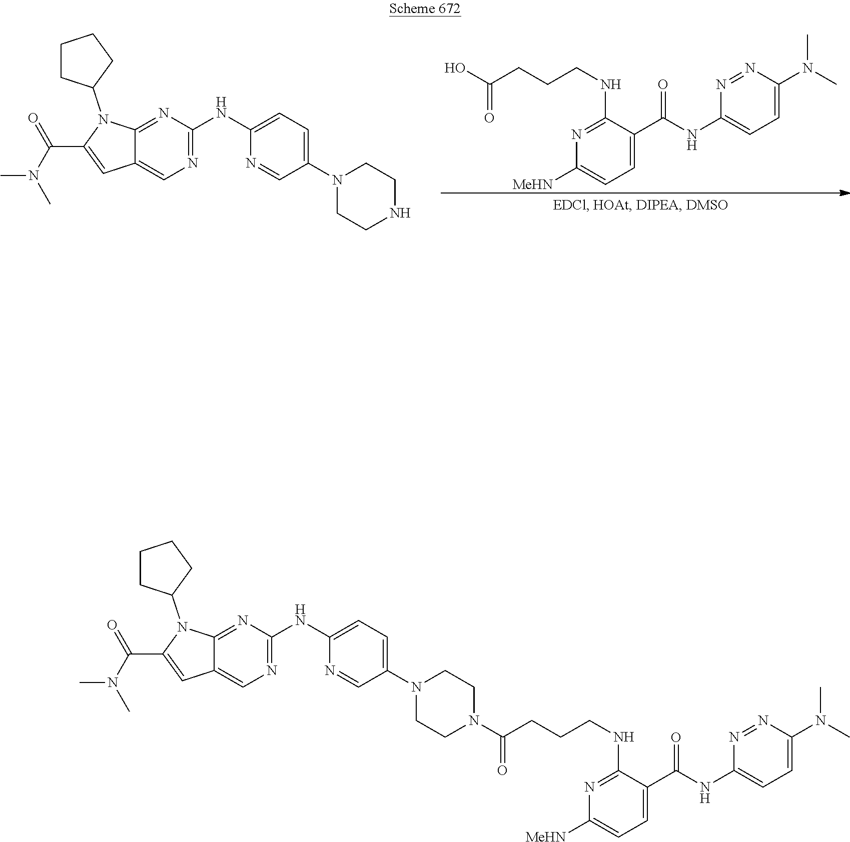

CPD-617 was synthesized following the standard procedure for preparing CPD-143 (2.0 mg, 24% yield) as a yellow solid. MS (ESI) m/z=790.6 [M+H]⁺.

### Example 673. 7-cyclopentyl-2-((5-(4-(5-((3-((6-(dimethylamino)pyridazin-3-yl)carbamoyl)-6-(methylamino)pyridin-2-yl)amino)pentanoyl)piperazin-1-yl)pyridin-2-yl)amino)-N,N-dimethyl-7H-pyrrolo[2,3-d]pyrimidine-6-carboxamide (CPD-618)

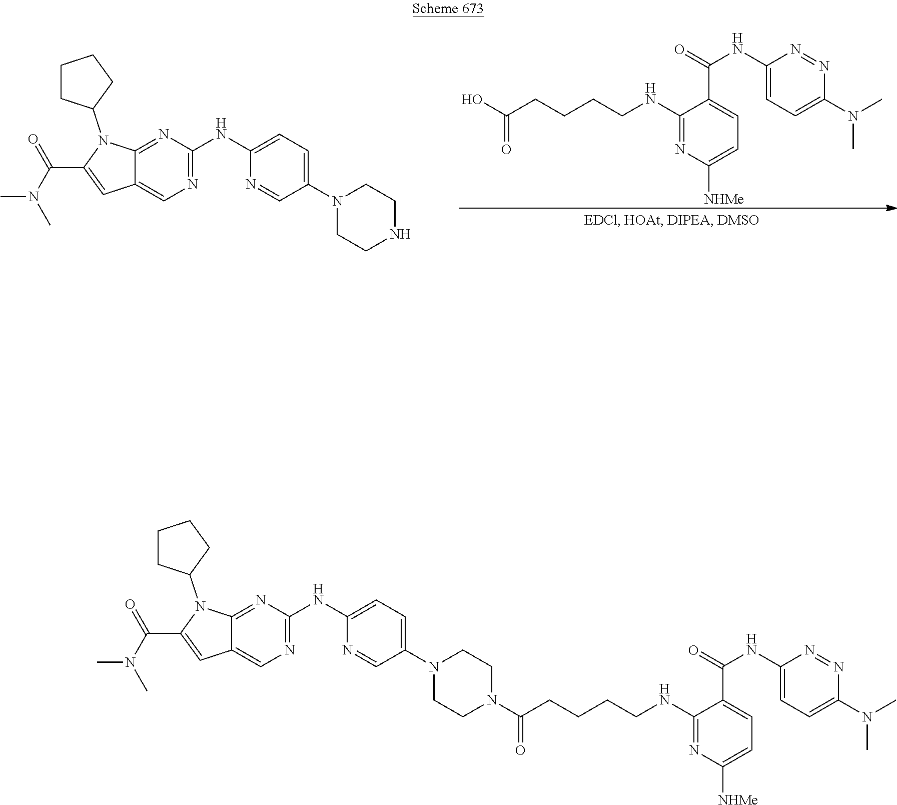

CPD-618 was synthesized following the standard procedure for preparing CPD-143 (2.6 mg, 30% yield) as a yellow solid. MS (ESI) m/z=804.7 [M+H]⁺.

### Example 674. 7-cyclopentyl-2-((5-(4-(5-((3-((6-(dimethylamino)pyridazin-3-yl)carbamoyl)-6-(methylamino)pyridin-2-yl)amino)pentanoyl)piperazin-1-yl)pyridin-2-yl)amino)-N,N-dimethyl-7H-pyrrolo[2,3-d]pyrimidine-6-carboxamide (CPD-619)

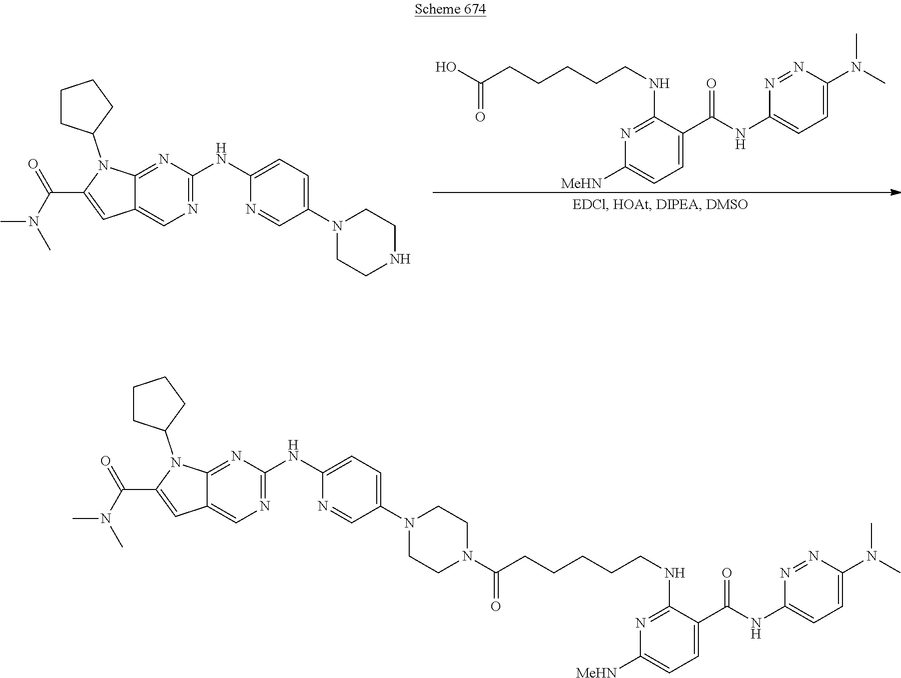

CPD-619 was synthesized following the standard procedure for preparing CPD-143 (2.7 mg, 31% yield) as a yellow solid. MS (ESI) m/z=818.7 [M+H]⁺.

### Example 675. 7-cyclopentyl-2-((5-(4-(7-((3-((6-(dimethylamino)pyridazin-3-yl)carbamoyl)-6-(methylamino)pyridin-2-yl)amino)heptanoyl)piperazin-1-yl)pyridin-2-yl)amino)-N,N-dimethyl-7H-pyrrolo[2,3-d]pyrimidine-6-carboxamide (CPD-620)

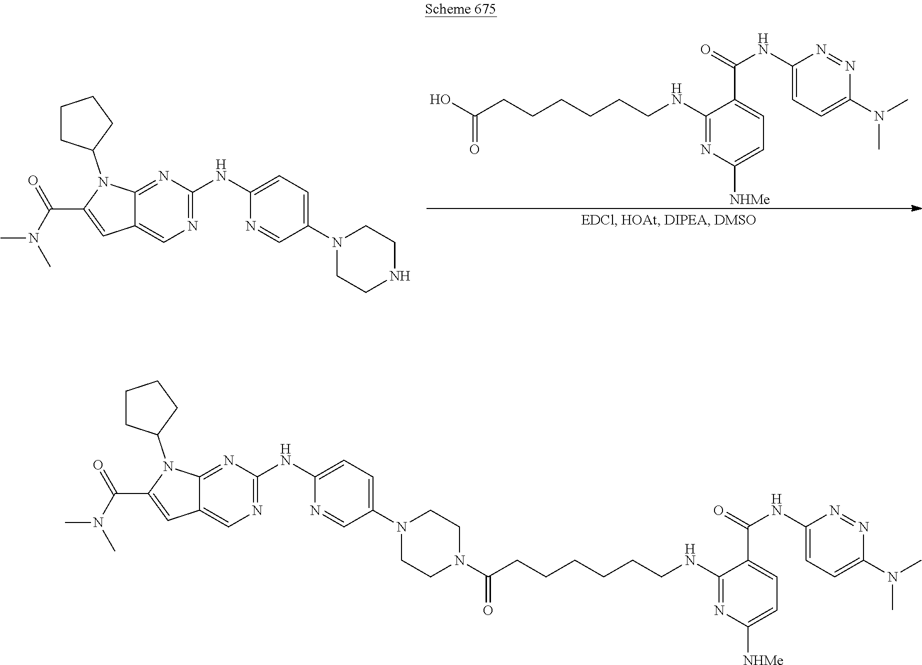

CPD-620 was synthesized following the standard procedure for preparing CPD-143 (2.7 mg, 36% yield) as a yellow solid. MS (ESI) m/z=832.7 [M+H]⁺.

### Example 676. 7-cyclopentyl-2-((5-(4-(8-((3-((6-(dimethylamino)pyridazin-3-yl)carbamoyl)-6-(methylamino)pyridin-2-yl)amino)octanoyl)piperazin-1-yl)pyridin-2-yl)amino)-N,N-dimethyl-7H-pyrrolo[2,3-d]pyrimidine-6-carboxamide (CPD-621)

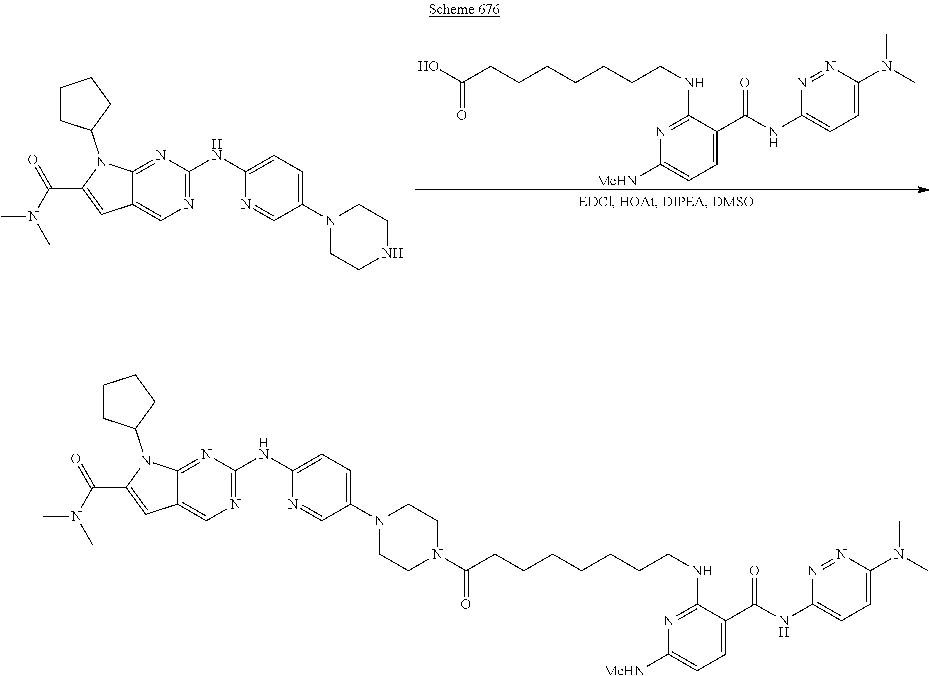

CPD-621 was synthesized following the standard procedure for preparing CPD-143 (2.4 mg, 31% yield) as a yellow solid. MS (ESI) m/z=846.7 [M+H]⁺.

### Example 677. 7-cyclopentyl-2-((5-(4-(3-(2-((3-((6-(dimethylamino)pyridazin-3-yl)carbamoyl)-6-(methylamino)pyridin-2-yl)amino)ethoxy)propanoyl)piperazin-1-yl)pyridin-2-yl)amino)-N,N-dimethyl-7H-pyrrolo[2,3-d]pyrimidine-6-carboxamide (CPD-622)

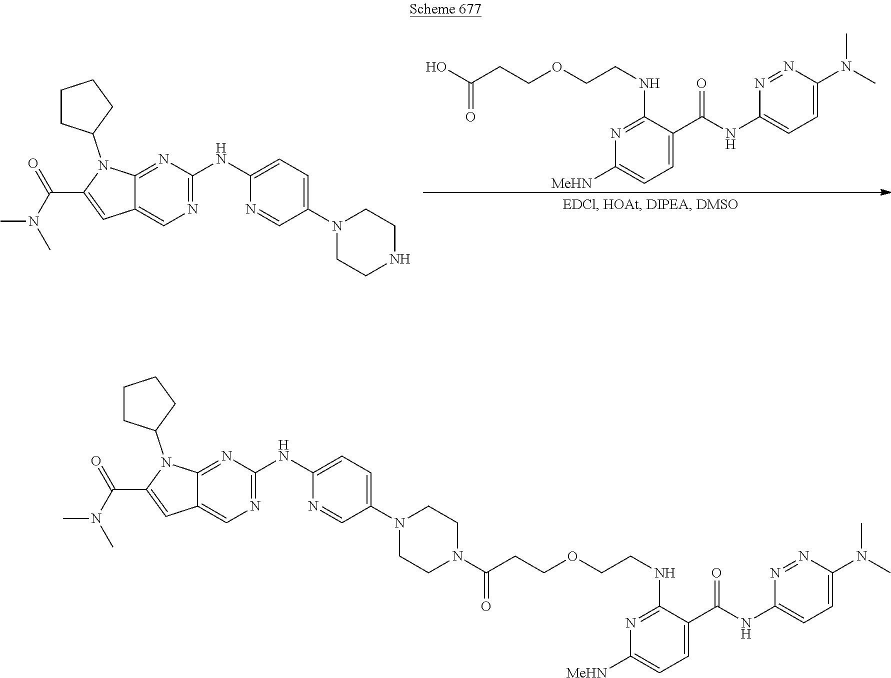

CPD-622 was synthesized following the standard procedure for preparing CPD-143 (1.7 mg, 20% yield) as a yellow solid. MS (ESI) m/z=820.6 [M+H]⁺.

### Example 678. 7-cyclopentyl-2-((5-(4-(3-(2-(2-((3-((6-(dimethylamino)pyridazin-3-yl)carbamoyl)-6-(methylamino)pyridin-2-yl)amino)ethoxy)ethoxy)propanoyl)piperazin-1-yl)pyridin-2-yl)amino)-N,N-dimethyl-7H-pyrrolo[2,3-d]pyrimidine-6-carboxamide (CPD-623)

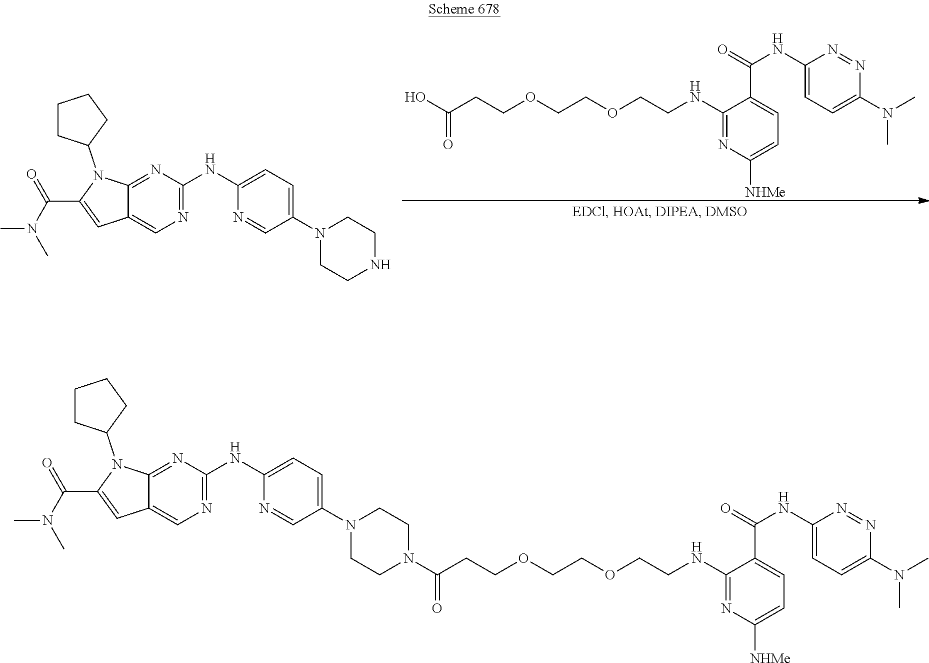

CPD-623 was synthesized following the standard procedure for preparing CPD-143 (2.6 mg, 29% yield) as a yellow solid. MS (ESI) m/z=864.6 [M+H]⁺.

### Example 679. 7-cyclopentyl-2-((5-(4-(2-((2-((3-((6-(dimethylamino)pyridazin-3-yl)carbamoyl)-6-(methylamino)pyridin-2-yl)amino)ethyl)amino)-2-oxoethyl)piperazin-1-yl)pyridin-2-yl)amino)-N,N-dimethyl-7H-pyrrolo[2,3-d]pyrimidine-6-carboxamide (CPD-624)

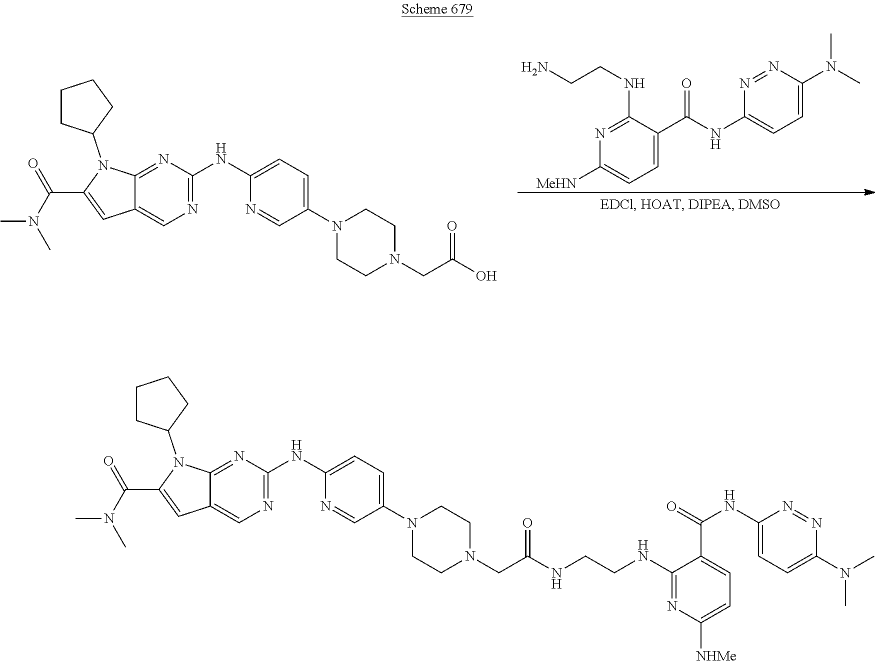

CPD-624 was synthesized following the standard procedure for preparing CPD-143 (2.1 mg, 30% yield) as a yellow solid. MS (ESI) m/z=805.6 [M+H]⁺.

### Example 680. 7-cyclopentyl-2-((5-(4-(2-((3-((3-((6-(dimethylamino)pyridazin-3-yl)carbamoyl)-6-(methylamino)pyridin-2-yl)amino)propyl)amino)-2-oxoethyl)piperazin-1-yl)pyridin-2-yl)amino)-N,N-dimethyl-7H-pyrrolo[2,3-d]pyrimidine-6-carboxamide (CPD-625)

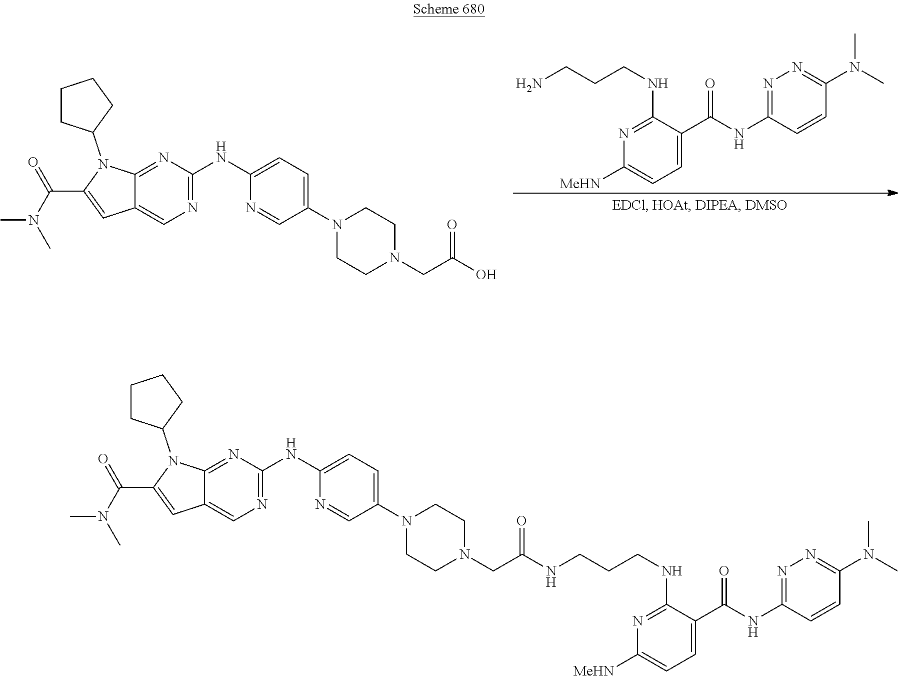

CPD-625 was synthesized following the standard procedure for preparing CPD-143 (2.7 mg, 40% yield) as a yellow solid. MS (ESI) m/z=819.6 [M+H]⁺.

### Example 681. 7-cyclopentyl-2-((5-(4-(2-((4-((3-((6-(dimethylamino)pyridazin-3-yl)carbamoyl)-6-(methylamino)pyridin-2-yl)amino)butyl)amino)-2-oxoethyl)piperazin-1-yl)pyridin-2-yl)amino)-N,N-dimethyl-7H-pyrrolo[2,3-d]pyrimidine-6-carboxamide (CPD-626)

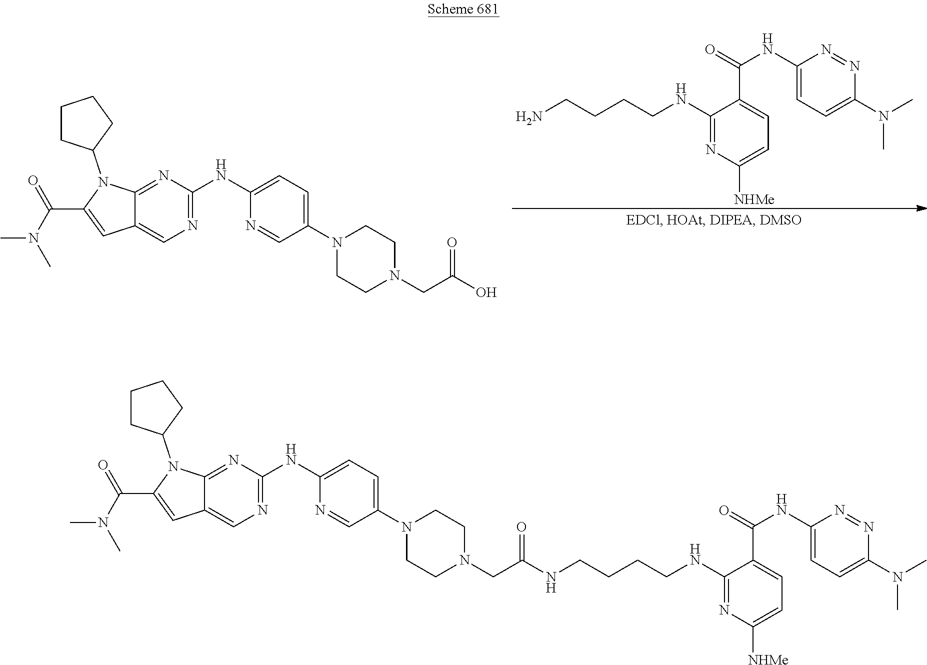

CPD-626 was synthesized following the standard procedure for preparing CPD-143 (2.8 mg, 36% yield) as a yellow solid. MS (ESI) mi/z=833.6 [M+H]⁺.

### Example 682. 7-cyclopentyl-2-((5-(4-(2-((5-((3-((6-(dimethylamino)pyridazin-3-yl)carbamoyl)-6-(methylamino)pyridin-2-yl)amino)pentyl)amino)-2-oxoethyl)piperazin-1-yl)pyridin-2-yl)amino)-N,N-dimethyl-7H-pyrrolo[2,3-d]pyrimidine-6-carboxamide (CPD-627)

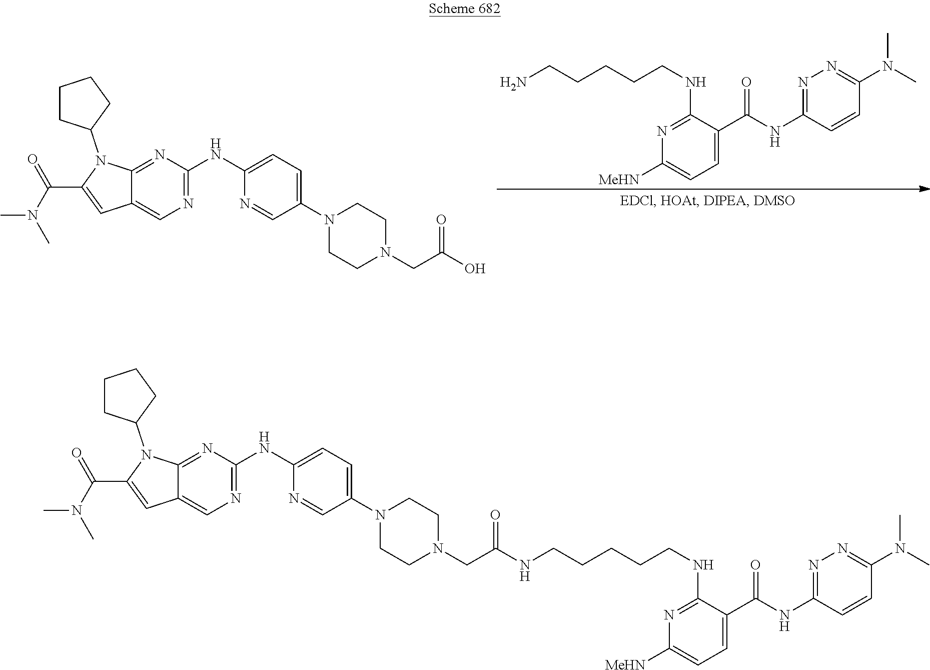

CPD-627 was synthesized following the standard procedure for preparing CPD-143 (3.1 mg, 39% yield) as a yellow solid. MS (ESI) m/z=847.7 [M+H]⁺.

### Example 683. 7-cyclopentyl-2-((5-(4-(2-((6-((3-((6-(dimethylamino)pyridazin-3-yl)carbamoyl)-6-(methylamino)pyridin-2-yl)amino)hexyl)amino)-2-oxoethyl)piperazin-1-yl)pyridin-2-yl)amino)-N,N-dimethyl-7H-pyrrolo[2,3-d]pyrimidine-6-carboxamide (CPD-628)

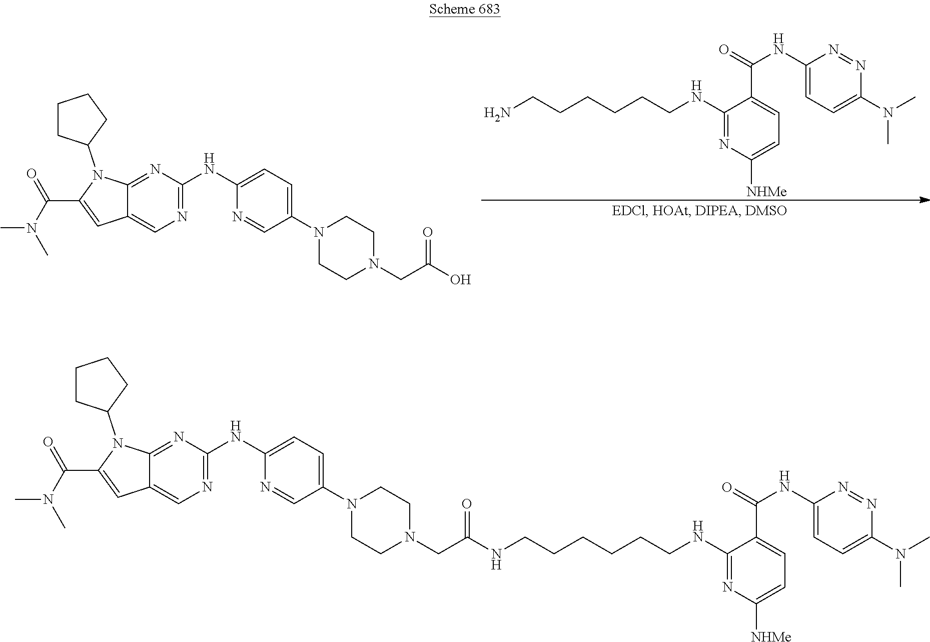

CPD-628 was synthesized following the standard procedure for preparing CPD-143 (2.4 mg, 30% yield) as a yellow solid. MS (ESI) m/z=861.6 [M+H]⁺.

### Example 684. 7-cyclopentyl-2-((5-(4-(2-((7-((3-((6-(dimethylamino)pyridazin-3-yl)carbamoyl)-6-(methylamino)pyridin-2-yl)amino)heptyl)amino)-2-oxoethyl)piperazin-1-yl)pyridin-2-yl)amino)-N,N-dimethyl-7H-pyrrolo[2,3-d]pyrimidine-6-carboxamide (CPD-629)

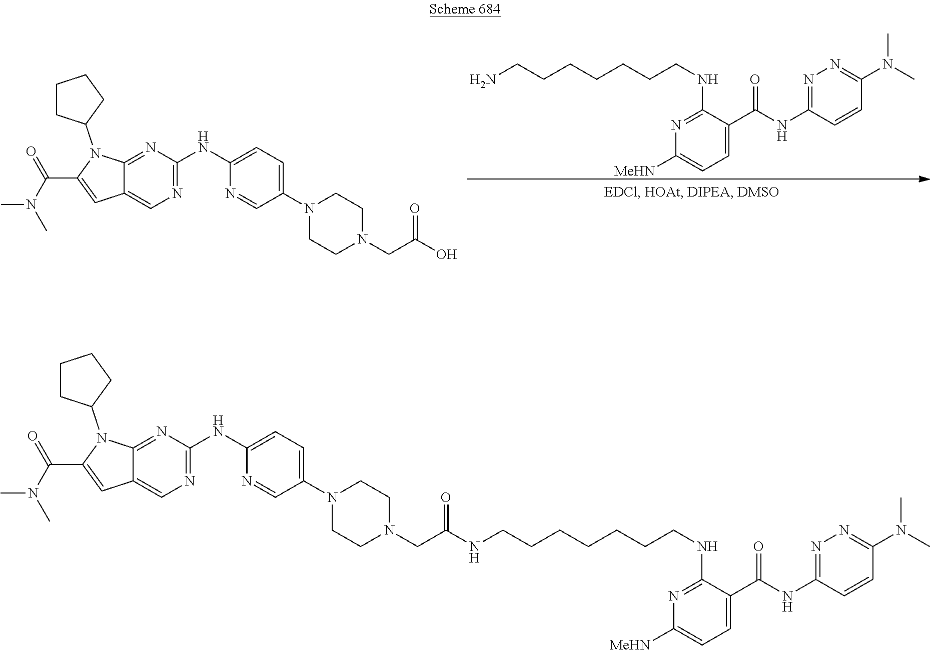

CPD-629 was synthesized following the standard procedure for preparing CPD-143 (2.9 mg, 40% yield) as a yellow solid. MS (ESI) m/z=875.6 [M+H]⁺.

### Example 685. 7-cyclopentyl-2-((5-(4-(2-((8-((3-((6-(dimethylamino)pyridazin-3-yl)carbamoyl)-6-(methylamino)pyridin-2-yl)amino)octyl) amino)-2-oxoethyl)piperazin-1-yl)pyridin-2-yl)amino)-N,N-dimethyl-7H-pyrrolo[2,3-d]pyrimidine-6-carboxamide (CPD-630)

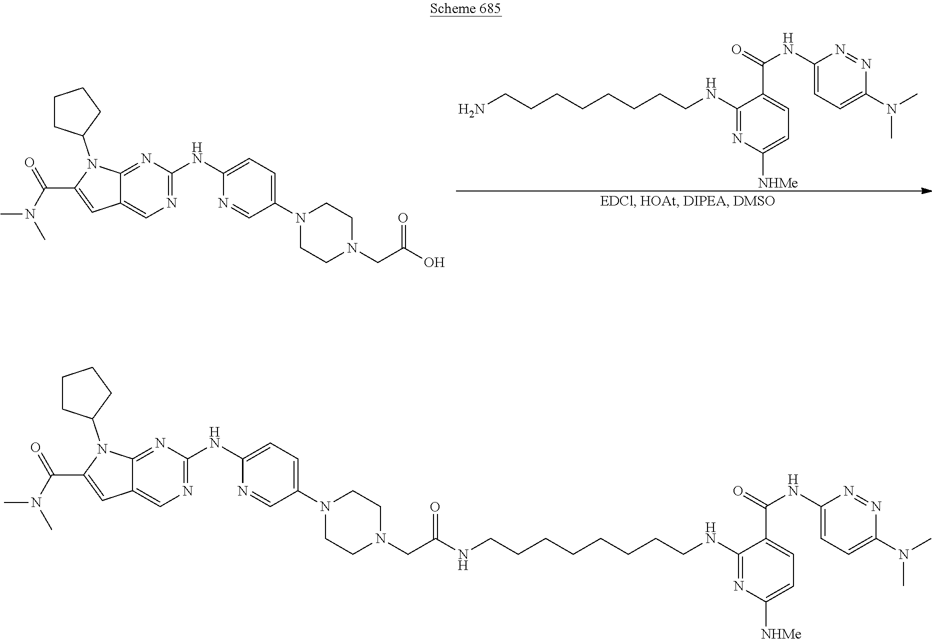

CPD-630 was synthesized following the standard procedure for preparing CPD-143 (2.4 mg, 29% yield) as a yellow solid. MS (ESI) m/z=889.6 [M+H]⁺.

### Example 686. 7-cyclopentyl-2-((5-(4-(2-((2-(2-((3-((6-(dimethylamino)pyridazin-3-yl)carbamoyl)-6-(methylamino)pyridin-2-yl)amino)ethoxy)ethyl)amino)-2-oxoethyl)piperazin-1-yl)pyridin-2-yl)amino)-N,N-dimethyl-7H-pyrrolo[2,3-d]pyrimidine-6-carboxamide (CPD-631)

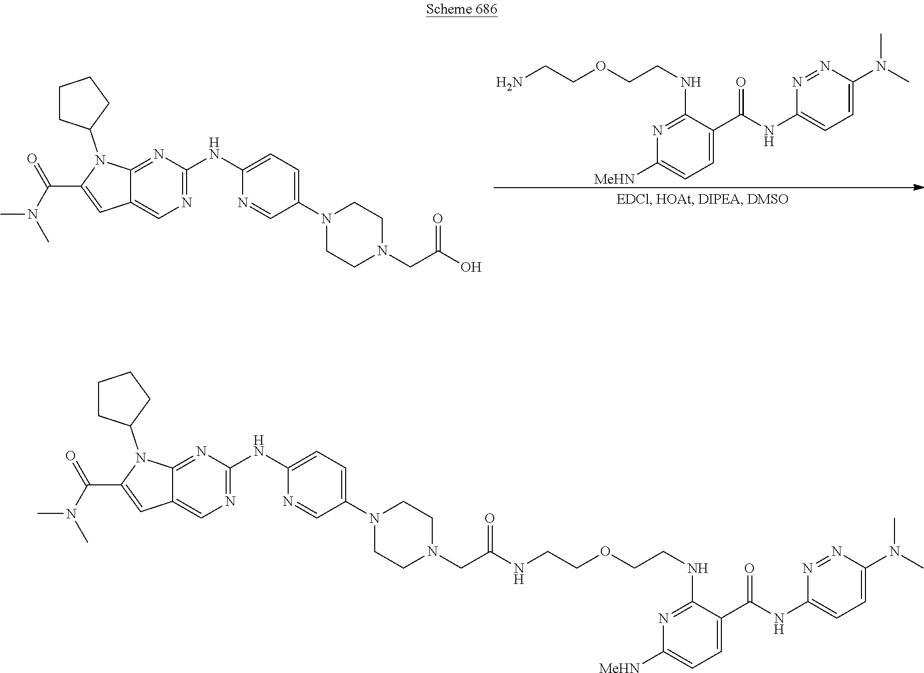

CPD-631 was synthesized following the standard procedure for preparing CPD-143 (2.3 mg, 29% yield) as a yellow solid. MS (ESI) m/z=849.6 [M+H]⁺.

### Example 687. N-(6-(dimethylamino)pyridazin-3-yl)-6-(methylamino)-2-((3-oxo-3-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)propyl)amino)nicotinamide (CPD-632)

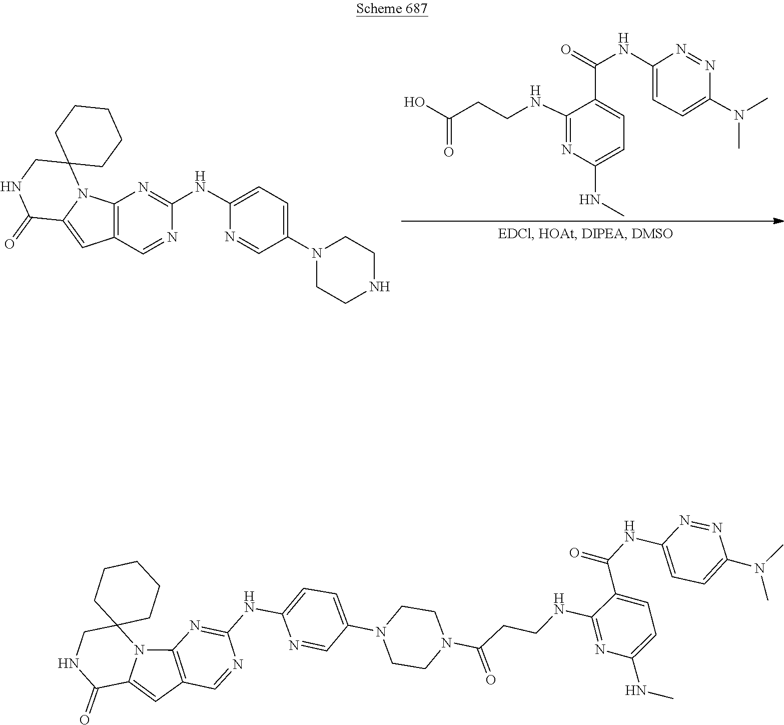 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCC(=O)O)n1 ; CNc1ccc(C(=O)Nc2ccc(N(C)C)nn2)c(NCCC(=O)N2CCN(c3ccc(Nc4ncc5cc6n(c5n4)C4(CCCCC4)CNC6=O)nc3)CC2)n1]

CPD-632 was synthesized following the standard procedure for preparing CPD-143 (2.2 mg, 25% yield) as a yellow solid. MS (ESI) m/z=774.8 [M+H]⁺.

### Example 688. N-(6-(dimethylamino)pyridazin-3-yl)-6-(methylamino)-2-((4-oxo-4-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amnion)pyridin-3-yl)piperazin-1-yl)butyl)amino)nicotinamide (CPD-633)

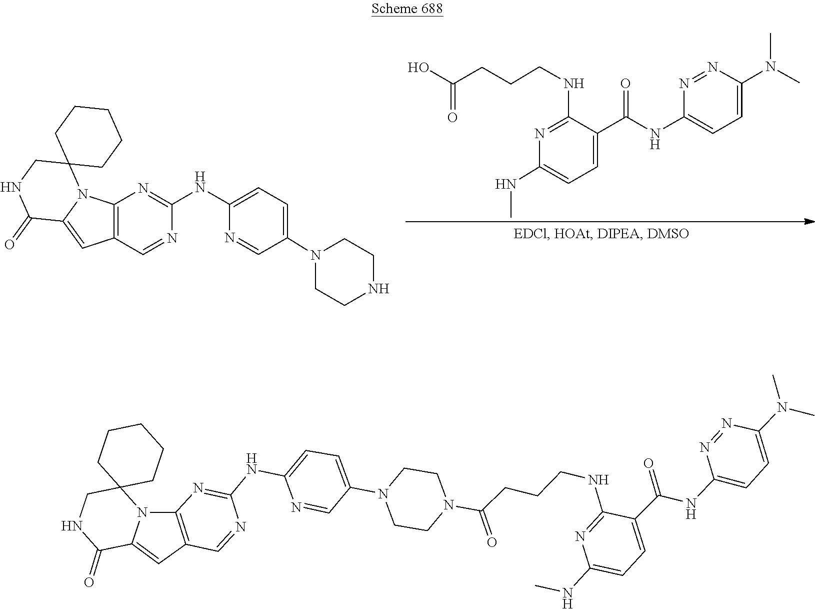

CPD-633 was synthesized following the standard procedure for preparing CPD-143 (2.5 mg, 28% yield) as a yellow solid. MS (ESI) m/z=788.7 [M+H]⁺.

### Example 689. N-(6-(dimethylamino)pyridazin-3-yl)-6-(methylamino)-2-((5-oxo-5-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)pentyl)amino)nicotinamide (CPD-634)

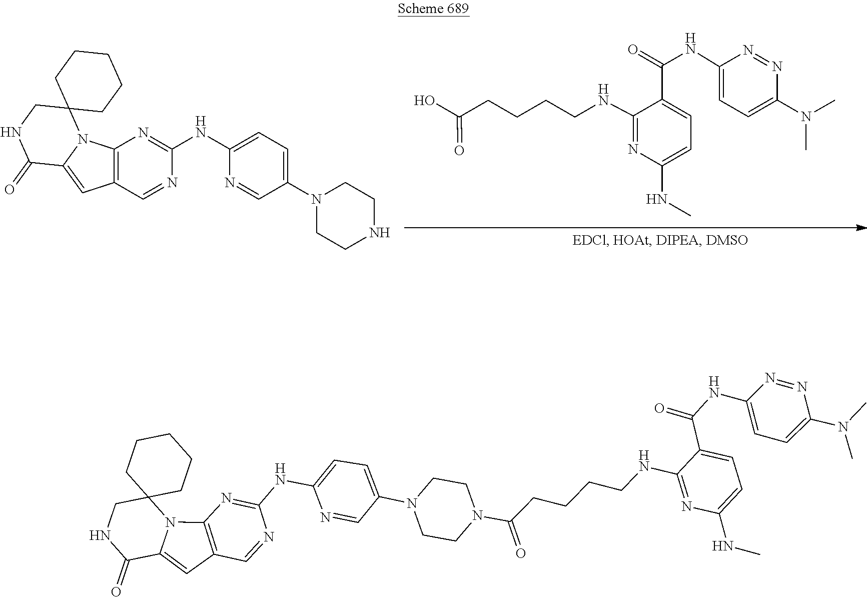 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCCCC(=O)O)n1 ; CNc1ccc(C(=O)Nc2ccc(N(C)C)nn2)c(NCCCCC(=O)N2CCN(c3ccc(Nc4ncc5cc6n(c5n4)C4(CCCCC4)CNC6=O)nc3)CC2)n1]

CPD-634 was synthesized following the standard procedure for preparing CPD-143 (2.5 mg, 27% yield) as a yellow solid. MS (ESI) m/z=802.6 [M+H]⁺.

### Example 690. N-(6-(dimethylamino)pyridazin-3-yl)-6-(methylamino)-2-((6-oxo-6-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)hexyl)amino)nicotinamide (CPD-635)

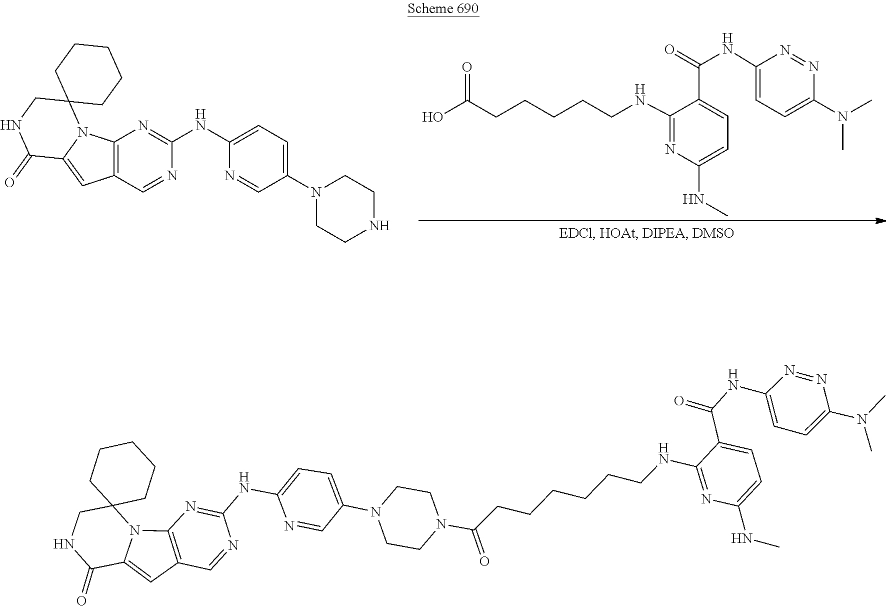 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCCCCC(=O)O)n1 ; CNc1ccc(C(=O)Nc2ccc(N(C)C)nn2)c(NCCCCCCC(=O)N2CCN(c3ccc(Nc4ncc5cc6n(c5n4)C4(CCCCC4)CNC6=O)nc3)CC2)n1]

CPD-635 was synthesized following the standard procedure for preparing CPD-143 (2.3 mg, 24% yield) as a yellow solid. MS (ESI) m/z=816.7 [M+H]⁺.

### Example 691. N-(6-(dimethylamino)pyridazin-3-yl)-6-(methylamino)-2-((7-oxo-7-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)heptyl)amino)nicotinamide (CPD-636)

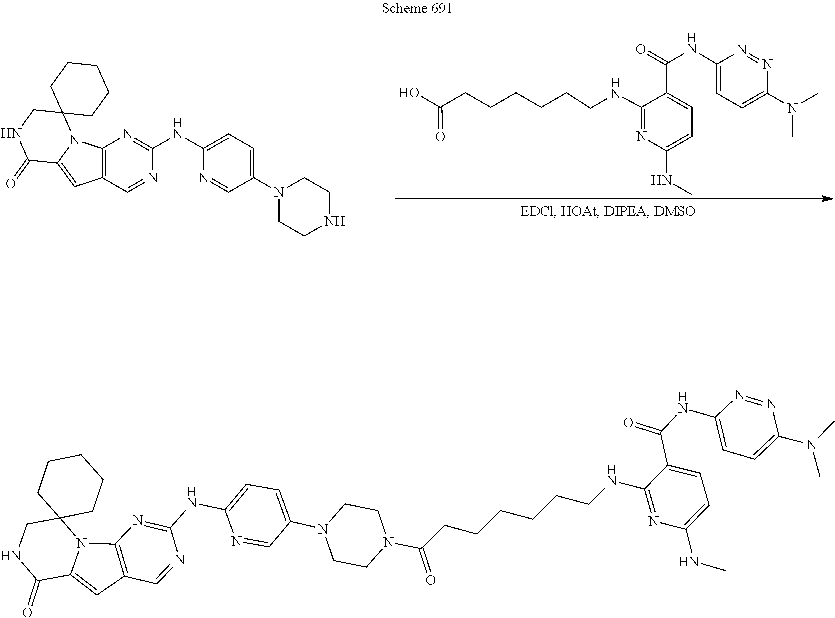 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCCCCCC(=O)O)n1 ; CNc1ccc(C(=O)Nc2ccc(N(C)C)nn2)c(NCCCCCCC(=O)N2CCN(c3ccc(Nc4ncc5cc6n(c5n4)C4(CCCCC4)CNC6=O)nc3)CC2)n1]

CPD-636 was synthesized following the standard procedure for preparing CPD-143 (2.6 mg, 27% yield) as a yellow solid. MS (ESI) m/z=830.7 [M+H]⁺.

### Example 692. N-(6-(dimethylamino)pyridazin-3-yl)-6-(methylamino)-2-((8-oxo-8-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)octyl)amino)nicotinamide (CPD-637)

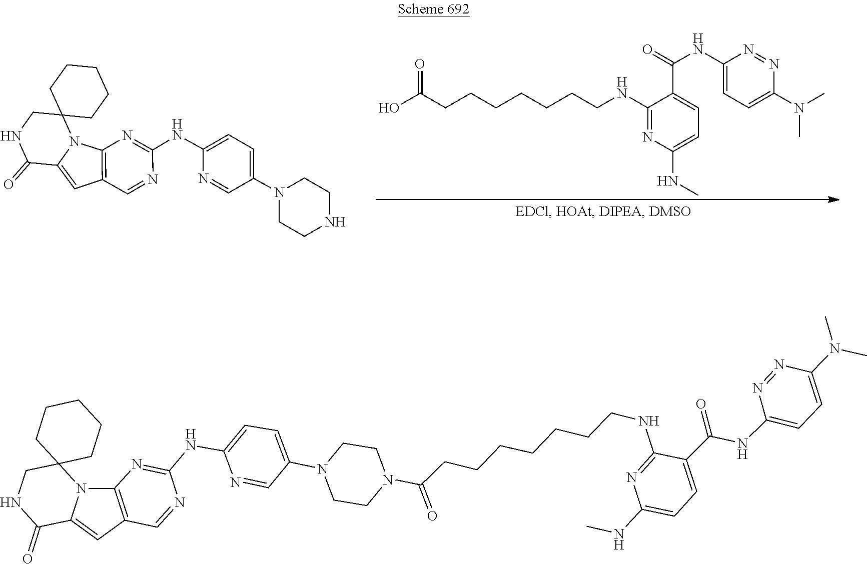 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCCCCCCC(=O)O)n1 ; CNc1ccc(C(=O)Nc2ccc(N(C)C)nn2)c(NCCCCCCCC(=O)N2CCN(c3ccc(Nc4ncc5cc6n(c5n4)C4(CCCCC4)CNC6=O)nc3)CC2)n1]

CPD-637 was synthesized following the standard procedure for preparing CPD-143 (2.2 mg, 23% yield) as a yellow solid. MS (ESI) m/z=844.7 [M+H]⁺.

### Example 693. N-(6-(dimethylamino)pyridazin-3-yl)-6-(methylamino)-2-((2-(3-oxo-3-(4-(6-((6′-oxo-7′,8′-dihydro-6′H-spiro[cyclohexane-1,9′-pyrazino[1′,2′:1,5]pyrrolo[2,3-d]pyrimidin-2′-yl)amino)pyridin-3-yl)piperazin-1-yl)propoxy)ethyl)amino)nicotinamide (CPD-638)

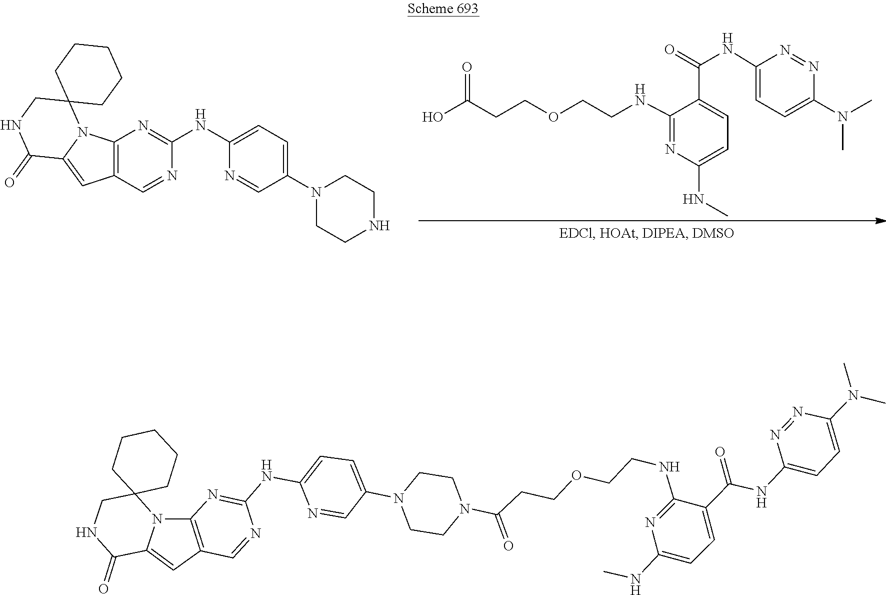

CPD-638 was synthesized following the standard procedure for preparing CPD-143 (5.4 mg, 57% yield) as a yellow solid. MS (ESI) m/z=818.7 [M+H]⁺.

### Example 694. N-(6-(dimethylamino)pyridazin-3-yl)-6-(methylamino)-2-((2-(2-(3-oxo-3-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)propoxy)ethoxy)ethyl)amino)nicotinamide (CPD-639)

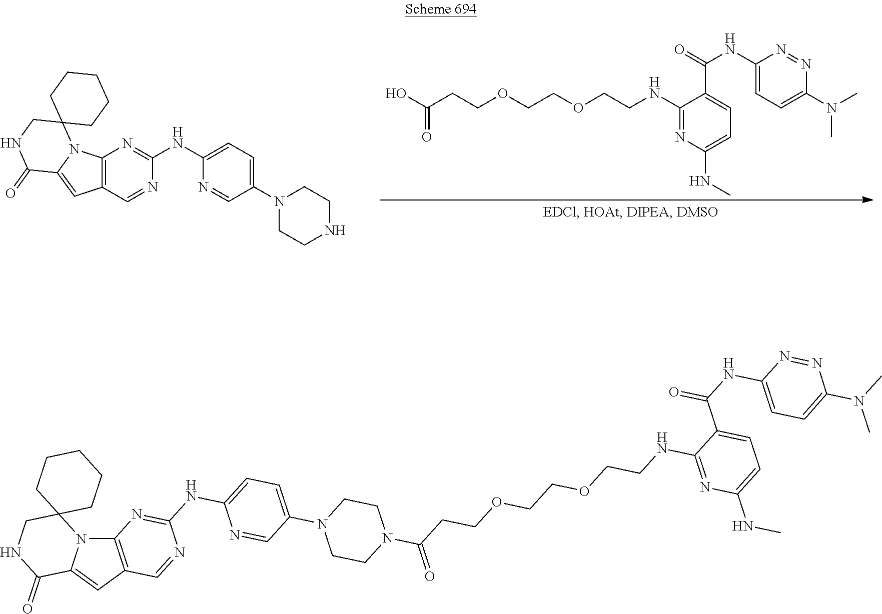 [SMILES: O=C1NCC2(CCCCC2)n2c1cc1cnc(Nc3ccc(N4CCNCC4)cn3)nc12 ; CNc1ccc(C(=O)Nc2ccc(N(C)C)nn2)c(NCCOCCOCCC(=O)O)n1 ; CNc1ccc(C(=O)Nc2ccc(N(C)C)nn2)c(NCCOCCOCCC(=O)N2CCN(c3ccc(Nc4ncc5cc6n(c5n4)C4(CCCCC4)CNC6=O)nc3)CC2)n1]

CPD-639 was synthesized following the standard procedure for preparing CPD-143 (5.5 mg, 55% yield) as a yellow solid. MS (ESI) m/z=862.7 [M+H]⁺.

### Example 695. N-(6-(dimethylamino)pyridazin-3-yl)-6-(methylamino)-2-((2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethyl)amino)nicotinamide (CPD-640)

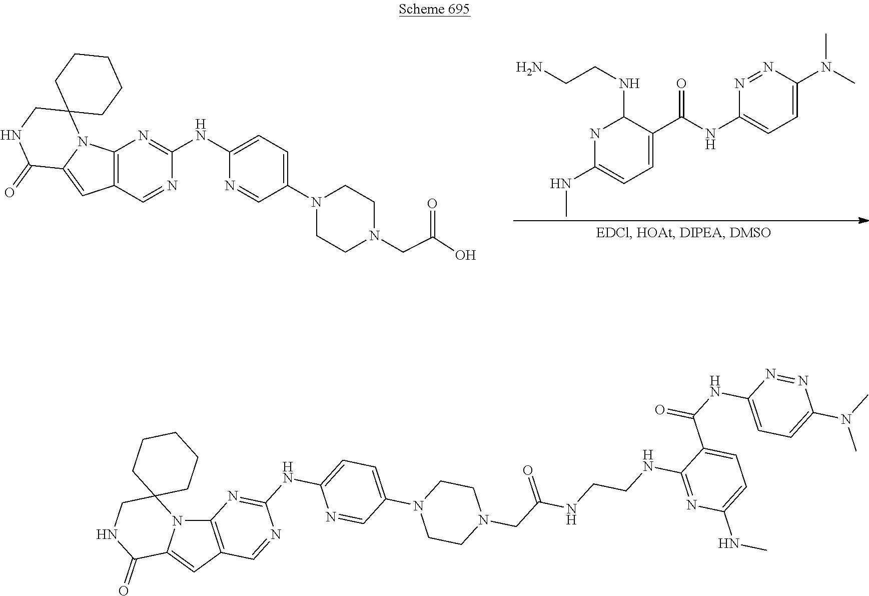

CPD-640 was synthesized following the standard procedure for preparing CPD-143 (2.1 mg, 27% yield) as a yellow solid. MS (ESI) m/z=803.7 [M+H]⁺.

### Example 696. N-(6-(dimethylamino)pyridazin-3-yl)-6-(methylamino)-2-((3-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)propyl)amino)nicotinamide (CPD-641)

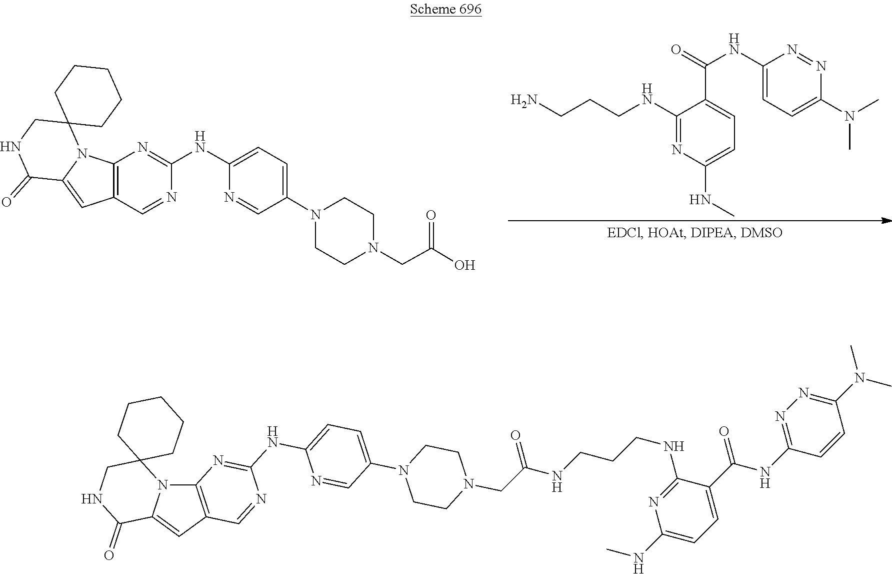 [SMILES: O=C(O)CN1CCN(c2ccc(Nc3ncc4cc5n(c4n3)C3(CCCCC3)CNC5=O)nc2)CC1 ; CNc1ccc(C(=O)Nc2ccc(N(C)C)nn2)c(NCCCN)n1 ; CNc1ccc(C(=O)Nc2ccc(N(C)C)nn2)c(NCCCNC(=O)CN2CCN(c3ccc(Nc4ncc5cc6n(c5n4)C4(CCCCC4)CNC6=O)nc3)CC2)n1]

CPD-641 was synthesized following the standard procedure for preparing CPD-143 (1.9 mg, 26% yield) as a yellow solid. MS (ESI) m/z=817.7 [M+H]⁺.

### Example 697. N-(6-(dimethylamino)pyridazin-3-yl)-6-(methylamino)-2-((4-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)butyl)amino)nicotinamide (CPD-642)

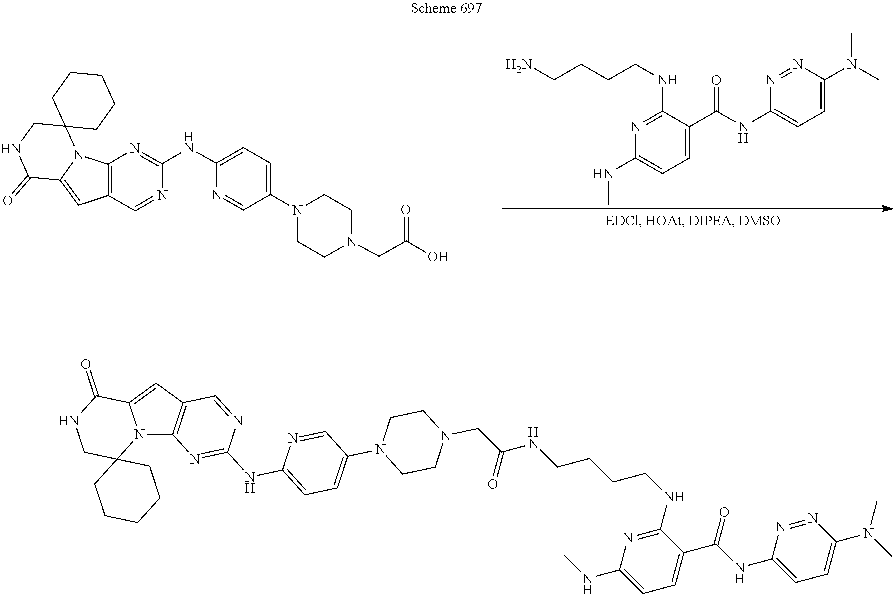 [SMILES: O=C(O)CN1CCN(c2ccc(Nc3ncc4cc5n(c4n3)C3(CCCCC3)CNC5=O)nc2)CC1 ; CNc1ccc(C(=O)Nc2ccc(N(C)C)nn2)c(NCCCCN)n1 ; CNc1ccc(C(=O)Nc2ccc(N(C)C)nn2)c(NCCCCNC(=O)CN2CCN(c3ccc(Nc4ncc5cc6n(c5n4)C4(CCCCC4)CNC6=O)nc3)CC2)n1]

CPD-642 was synthesized following the standard procedure for preparing CPD-143 (2.3 mg, 31% yield) as a yellow solid. MS (ESI) m/z=831.7 [M+H]⁺.

### Example 698. N-(6-(dimethylamino)pyridazin-3-yl)-6-(methylamino)-2-((5-(2-(4-(6-((6′-oxo-7′,8′-dihydro-6′H-spiro[cyclohexane-1,9′-pyrazino[1′,2′:1,5]pyrrolo[2,3-d pyrimidin]-2′-yl)amino)pyridin-3-yl)piperazin-1-yl)acetamido)pentyl)amino)nicotinamide (CPD-643)

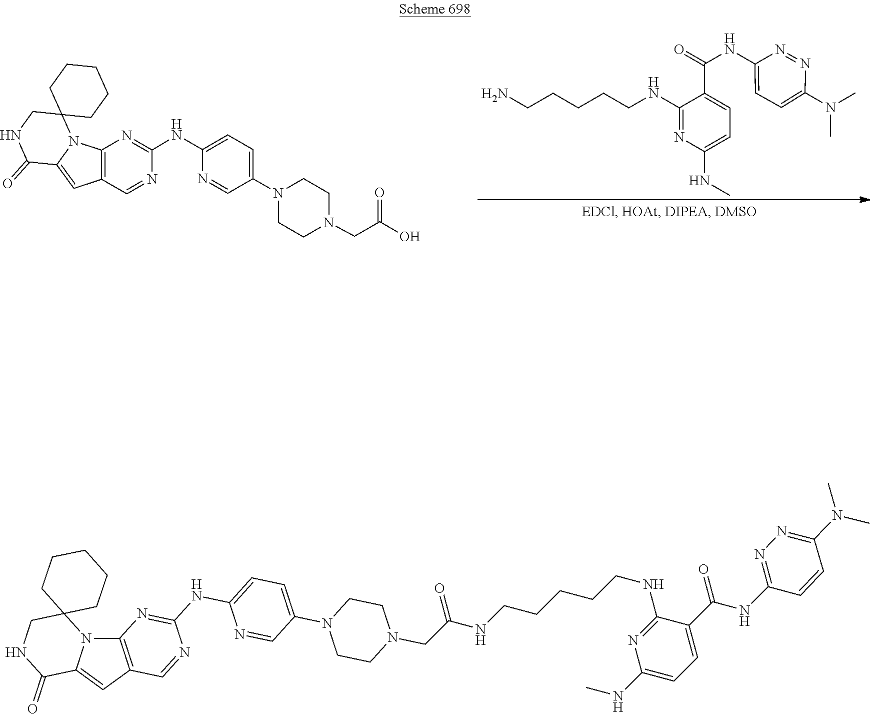

CPD-643 was synthesized following the standard procedure for preparing CPD-143 (2.2 mg, 28% yield) as a yellow solid. MS (ESI) m/z=845.8 [M+H]⁺.

### Example 699. N-(6-(dimethylamino)pyridazin-3-yl)-6-(methylamino)-2-((6-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)hexyl)amino)nicotinamide (CPD-644)

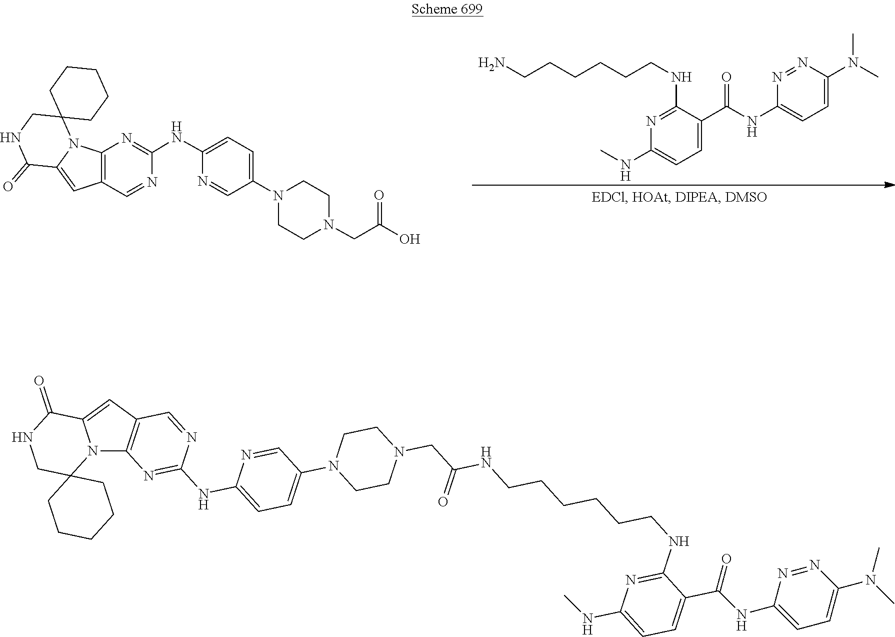 [SMILES: O=C(O)CN1CCN(c2ccc(Nc3ncc4cc5n(c4n3)C3(CCCCC3)CNC5=O)nc2)CC1 ; CNc1ccc(C(=O)Nc2ccc(N(C)C)nn2)c(NCCCCCCN)n1 ; CNc1ccc(C(=O)Nc2ccc(N(C)C)nn2)c(NCCCCCCNC(=O)CN2CCN(c3ccc(Nc4ncc5cc6n(c5n4)C4(CCCCC4)CNC6=O)nc3)CC2)n1]

CPD-644 was synthesized following the standard procedure for preparing CPD-143 (5.4 mg, 69% yield) as a yellow solid. MS (ESI) m/z=859.8 [M+H]⁺.

### Example 700. N-(6-(dimethylamino)pyridazin-3-yl)-6-(methylamino)-2-((7-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)heptyl)amino)nicotinamide (CPD-645)

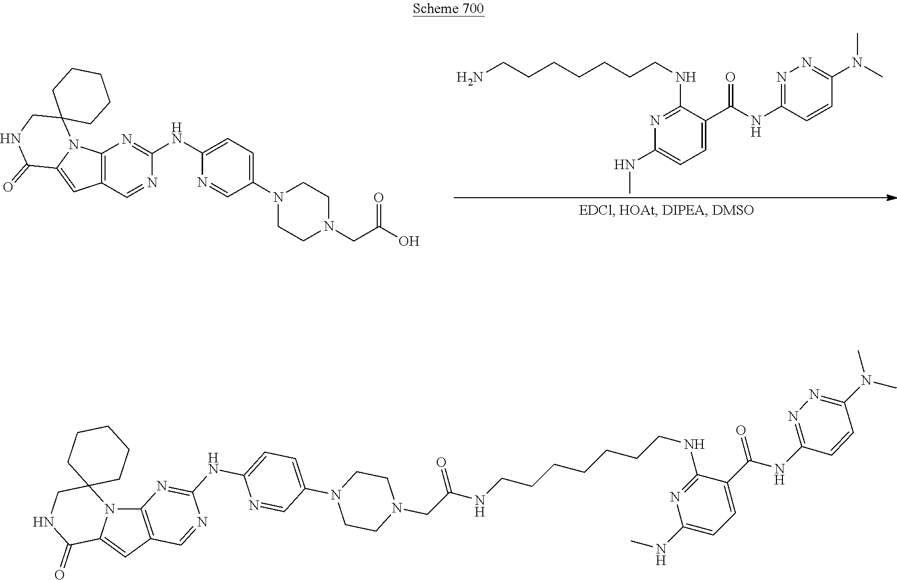

CPD-645 was synthesized following the standard procedure for preparing CPD-143 (1.8 mg, 23% yield) as a yellow solid. MS (ESI) m/z=873.8 [M+H]⁺.

### Example 701. N-(6-(dimethylamino)pyridazin-3-yl)-6-(methylamino)-2-((8-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)octyl)amino)nicotinamide (CPD-646)

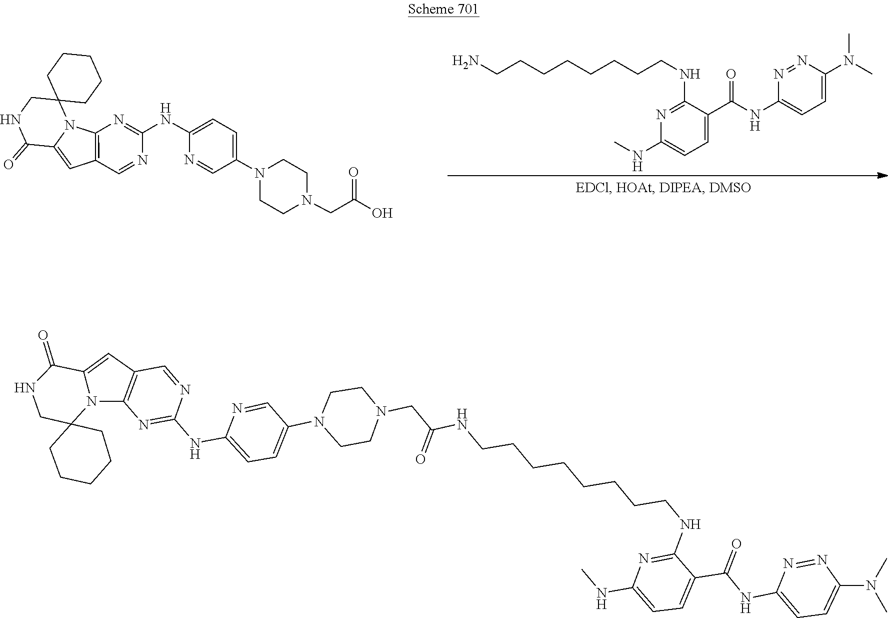 [SMILES: O=C(O)CN1CCN(c2ccc(Nc3ncc4cc5n(c4n3)C3(CCCCC3)CNC5=O)nc2)CC1 ; CNc1ccc(C(=O)Nc2ccc(N(C)C)nn2)c(NCCCCCCCCN)n1 ; CNc1ccc(C(=O)Nc2ccc(N(C)C)nn2)c(NCCCCCCCCNC(=O)CN2CCN(c3ccc(Nc4ncc5cc6n(c5n4)C4(CCCCC4)CNC6=O)nc3)CC2)n1]

CPD-646 was synthesized following the standard procedure for preparing CPD-143 (2.4 mg, 29% yield) as a yellow solid. MS (ESI) m/z=887.8 [M+H]⁺.

### Example 702. N-(6-(dimethylamino)pyridazin-3-yl)-6-(methylamino)-2-((2-(2-(2-(4-(6-((6′-oxo-7′,8′-dihydro-6′H-spiro[cyclohexane-1,9′-pyrazino[1′,2′:1,5]pyrrolo[2,3-d]pyrimidin]-2′-yl)amino)pyridin-3-yl)piperazin-1-yl)acetamido)ethoxy)ethyl)amino)nicotinamide (CPD-647)

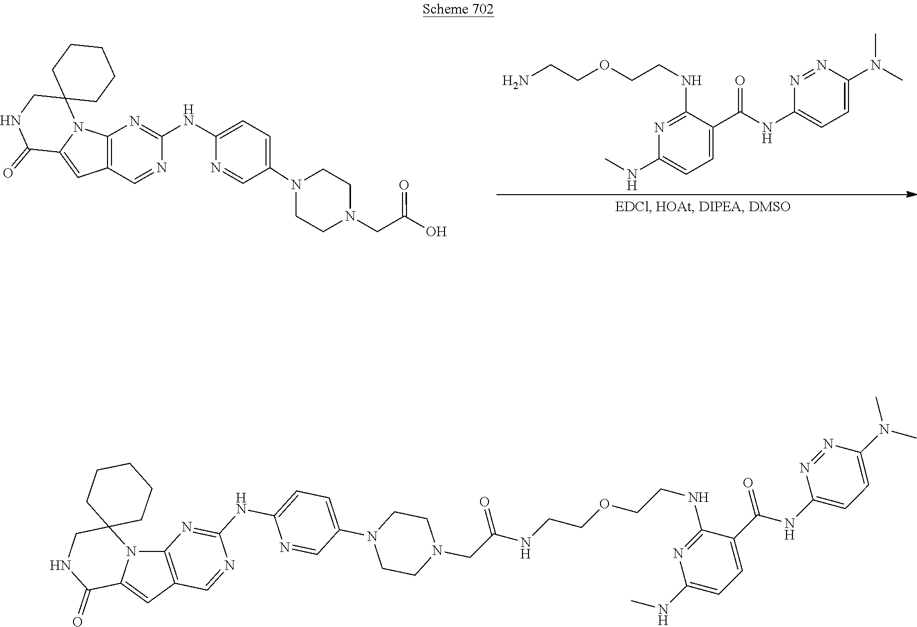

CPD-647 was synthesized following the standard procedure for preparing CPD-143 (1.8 mg, 23% yield) as a yellow solid. MS (ESI) m/z=847.7 [M+H]⁺.

### Example 703. N-(6-(dimethylamino)pyridazin-3-yl)-2-((3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropyl)amino)-6-(methylamino)nicotinamide (CPD-648)

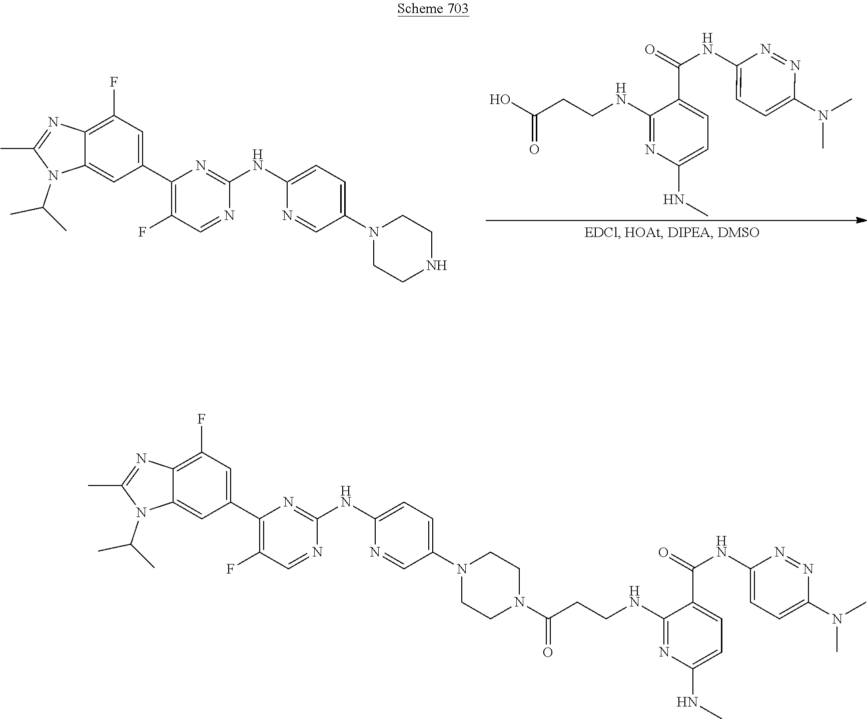

CPD-648 was synthesized following the standard procedure for preparing CPD-143 (9.4 mg, 68% yield) as a yellow solid. MS (ESI) m/z=806.9 [M+H]⁺.

### Example 704. N-(6-(dimethylamino)pyridazin-3-yl)-2-((4-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-4-oxobutyl)amino)-6-(methylamino)nicotinamide (CPD-649)

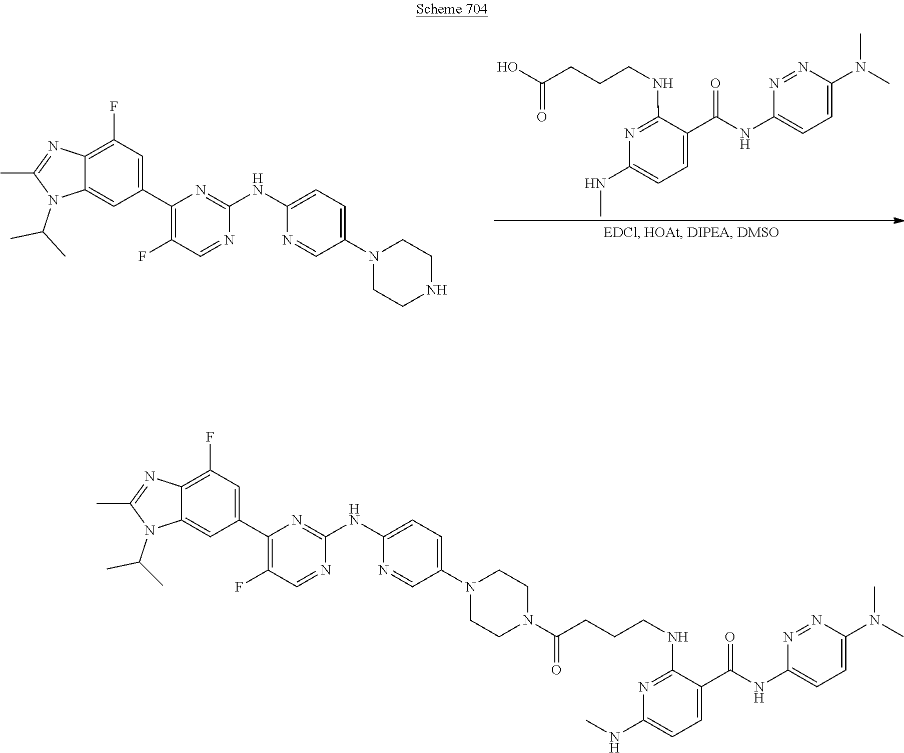

CPD-649 was synthesized following the standard procedure for preparing CPD-143 (2.0 mg, 14% yield) as a yellow solid. MS (ESI) m/z=820.9 [M+H]⁺.

### Example 705. N-(6-(dimethylamino)pyridazin-3-yl)-2-((5-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-5-oxopentyl)amino)-6-(methylamino)nicotinamide (CPD-650)

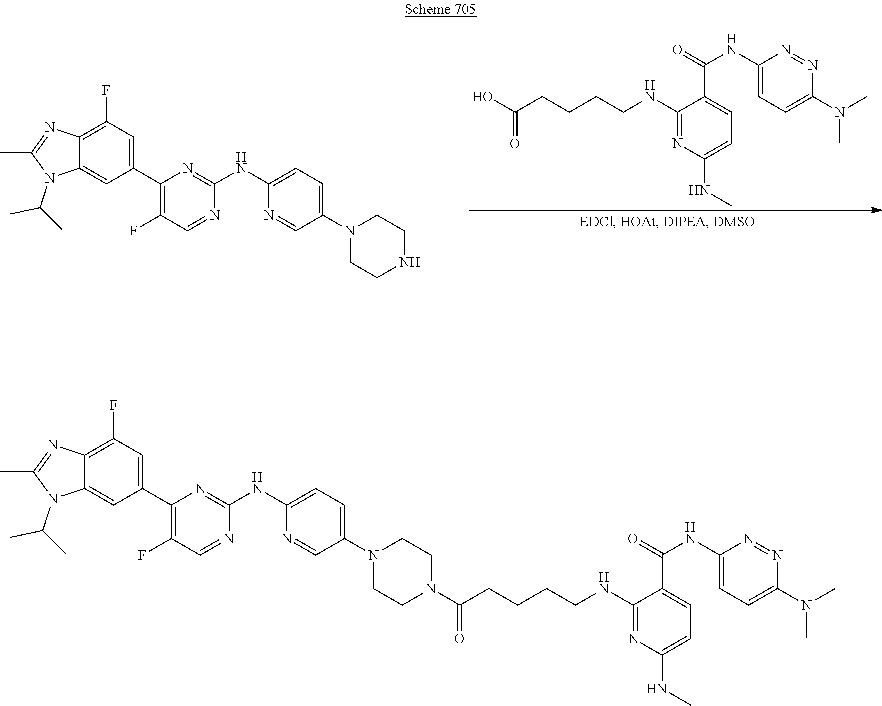

CPD-650 was synthesized following the standard procedure for preparing CPD-143 (4.3 mg, 30% yield) as a yellow solid. MS (ESI) m/z=834.9 [M+H]⁺.

### Example 706. N-(6-(dimethylamino)pyridazin-3-yl)-2-((6-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-6-oxohexyl)amino)-6-(methylamino)nicotinamide (CPD-651)

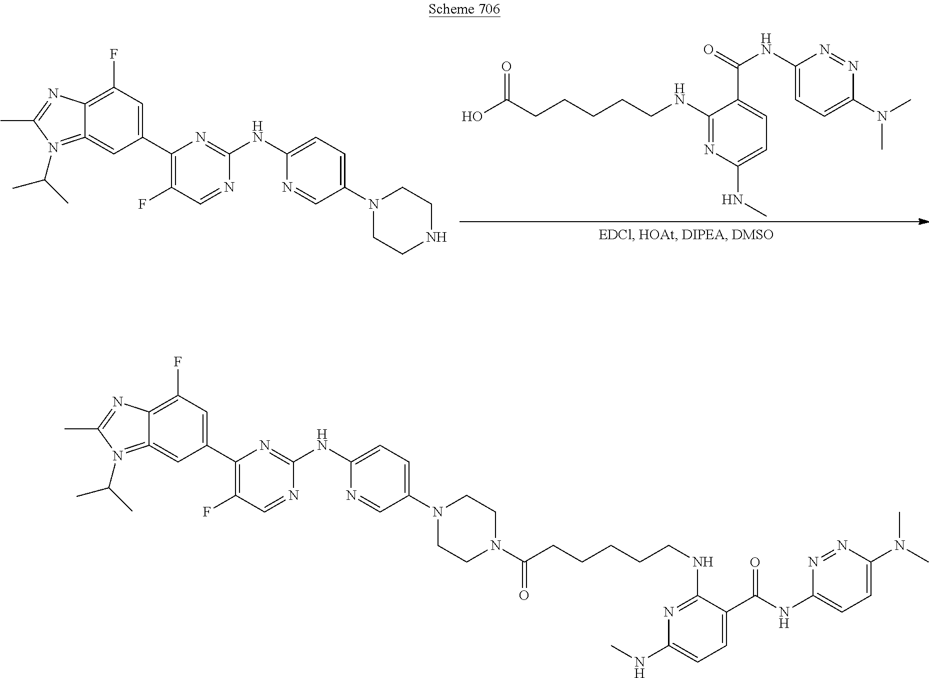

CPD-651 was synthesized following the standard procedure for preparing CPD-143 (2.2 mg, 15% yield) as a yellow solid. MS (ESI) m/z=849.0 [M+H]⁺.

### Example 707. N-(6-(dimethylamino)pyridazin-3-yl)-2-((7-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-7-oxoheptyl)amino)-6-(methylamino)nicotinamide (CPD-652)

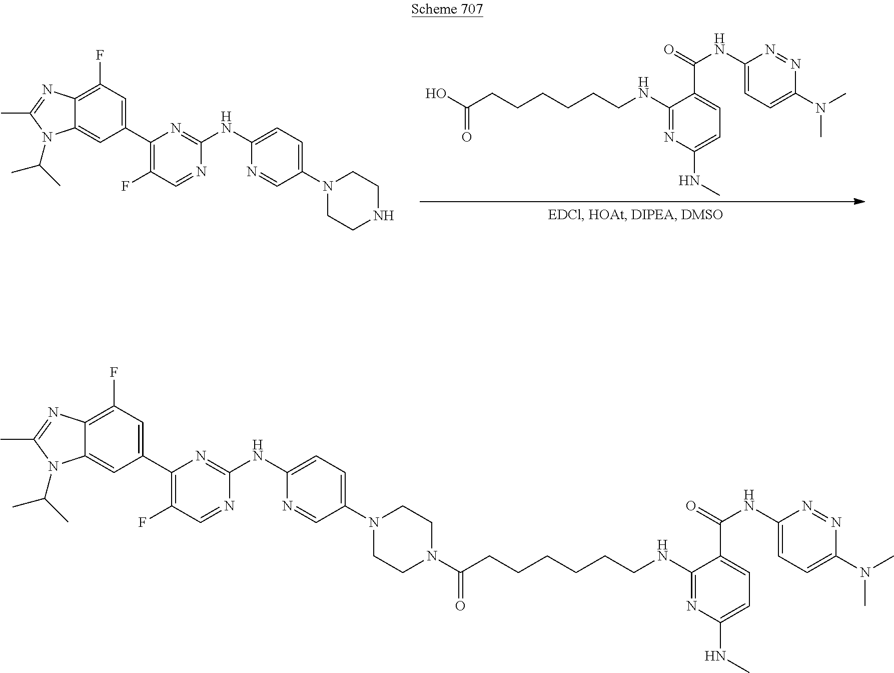

CPD-652 was synthesized following the standard procedure for preparing CPD-143 (6.9 mg, 47% yield) as a yellow solid. MS (ESI) m/z=862.9 [M+H]⁺.

### Example 708. N-(6-(dimethylamino)pyridazin-3-yl)-2-((8-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-8-oxooctyl)amino)-6-(methylamino)nicotinamide (CPD-653)

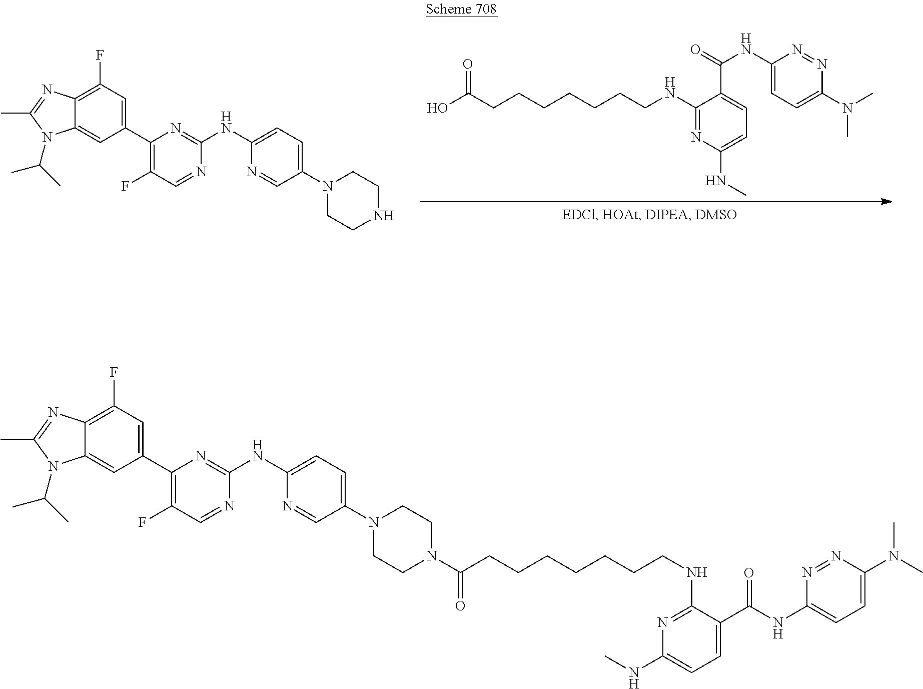

CPD-653 was synthesized following the standard procedure for preparing CPD-143 (6.4 mg, 43% yield) as a yellow solid. MS (ESI) m/z=876.2 [M+H]⁺.

### Example 709. N-(6-(dimethylamino)pyridazin-3-yl)-2-((2-(3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropoxy)ethyl)amino)-6-(methylamino)nicotinamide (CPD-654)

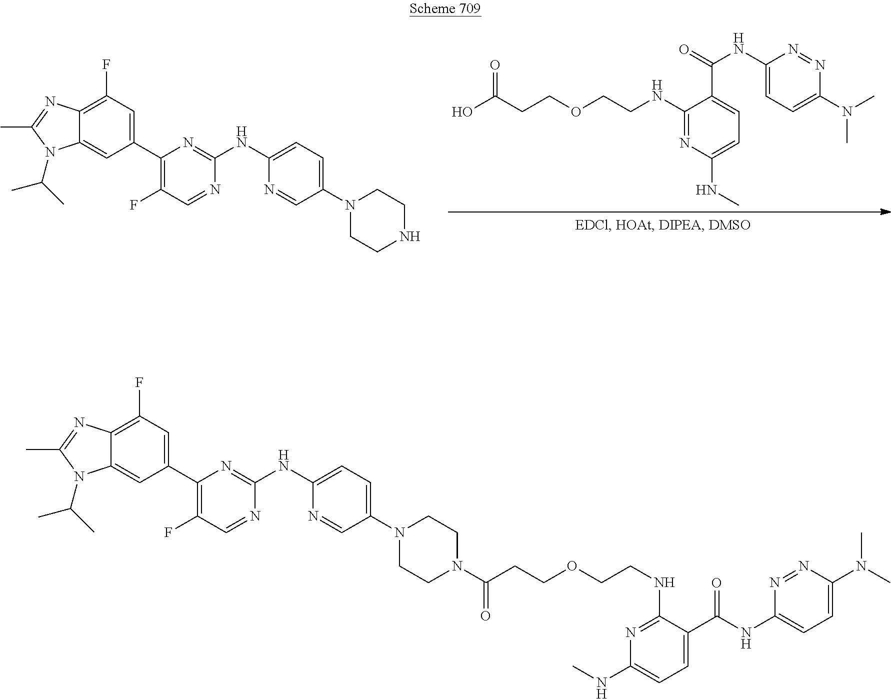

CPD-654 was synthesized following the standard procedure for preparing CPD-143 (8.6 mg, 59% yield) as a yellow solid. MS (ESI) m/z=850.9 [M+H]⁺.

### Example 710. N-(6-(dimethylamino)pyridazin-3-yl)-2-((2-(2-(3-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)-3-oxopropoxy)ethoxy)ethyl)amino)-6-(methylamino)nicotinamide (CPD-655)

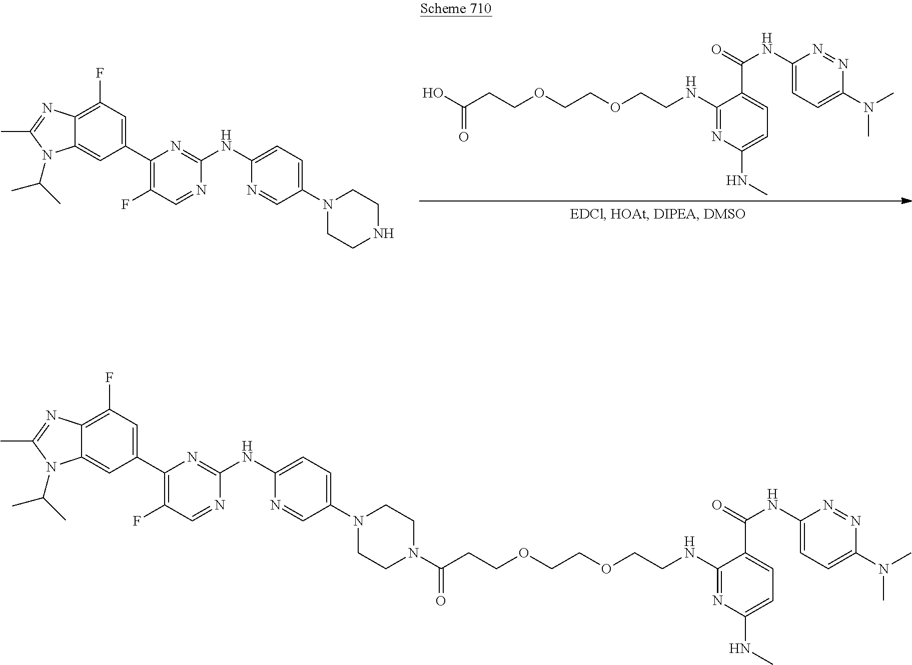

CPD-655 was synthesized following the standard procedure for preparing CPD-143 (5.5 mg, 36% yield) as a yellow solid. MS (ESI) m/z=895.0 [M+H]⁺.

### Example 711. N-(6-(dimethylamino)pyridazin-3-yl)-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)-6-(methylamino)nicotinamide (CPD-656)

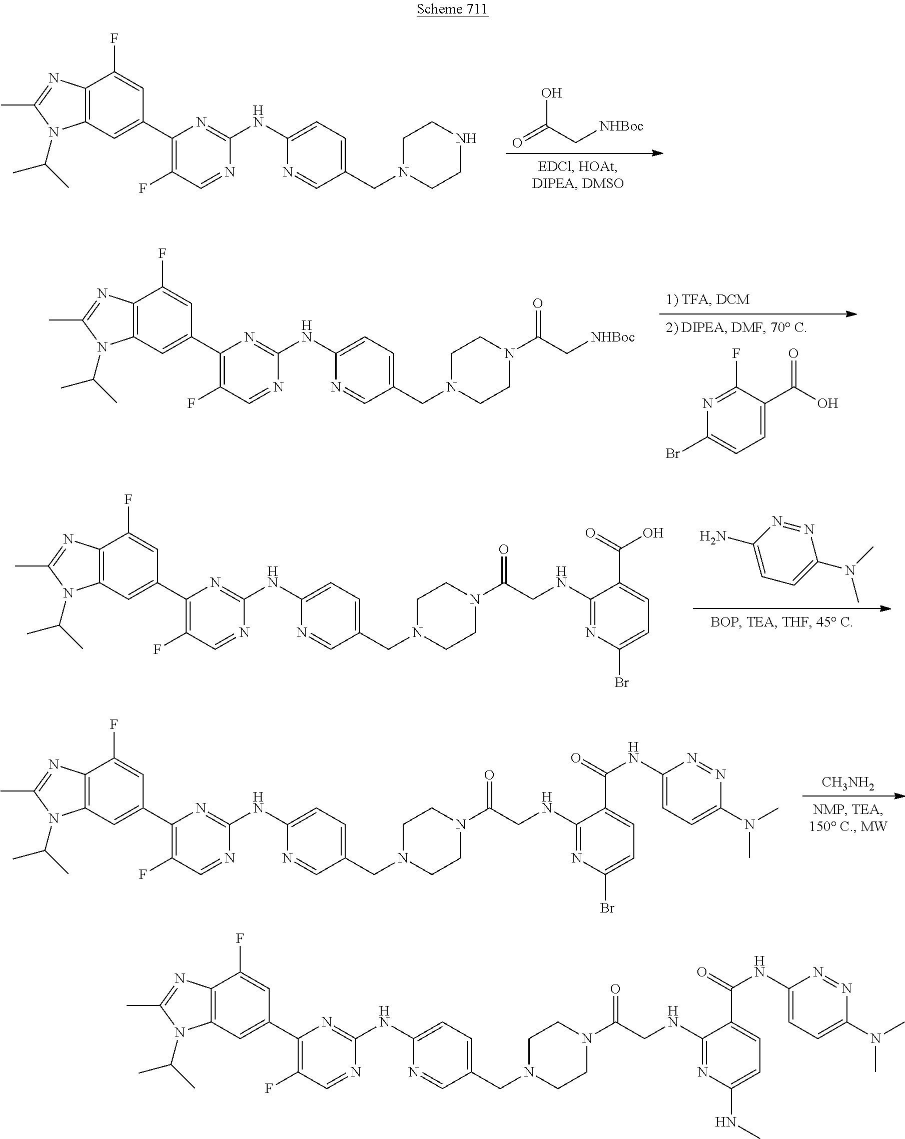

### Step 1. Synthesis of tert-butyl (2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)carbamate

To a mixture of 5-fluoro-4-(4-fluoro-1-isopropyl-2-methyl-1H-benzo[d]imidazol-6-yl)-N-(5-(piperazin-1-ylmethyl)pyridin-2-yl)pyrimidin-2-amine (70 mg, 0.15 mmol), DIPEA (189 mg, 1.46 mmol) and HOAt (41 mg, 0.29 mmol) in DMSO (3 mL) were added EDCI (56.08 mg, 0.29 mmol) and (tert-butoxycarbonyl)glycine (51 mg, 0.29 mmol) at rt. The mixture was stirred at rt for 15 h, before it was purified by reverse-phase chromatography to provide the desired product (70 mg, 75% yield) as a white solid. MS (ESI) m/z=636.4 [M+H]⁺.

### Step 2. Synthesis of 2-amino-1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethan-1-one

To a solution of tert-butyl (2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)carbamate (70 mg, 0. 11 mmol) in dichloromethane (5 mL) was added TFA (2 mL) at rt. The mixture was stirred at rt for 1 h. Then the solution was concentrated under reduced pressure to provide the crude product (60 mg, 84% yield) as a yellow solid. This compound was used directly in the next step without further purification. MS (ESI) m/z=536.4[M+H]⁺.

### Step 3. Synthesis of 6-bromo-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)nicotinic acid

To a mixture of 2-amino-1-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)ethan-1-one (60 mg, 0.11 mmol) in DMF (5 mL) were added 6-bromo-2-fluoronicotinic acid (24 mg, 0.11 mmol) and DIPEA (129 mg, 1 mol). The mixture was stirred at 90° C. for 18 h. The reaction mixture was diluted with water (30 mL) and extracted with dichloromethane (3×30 mL). The combined organic layers were washed with brine, dried with anhydrous sodium sulfate, filtered, and concentrated. The residue was purified by prep-HPLC to provide the desired product (50 mg, 53% yield) as a white solid. MS (ESI) m/z=736.0 [M+H]⁺.

### Step 4. Synthesis of 6-bromo-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)nicotinic acid

To a mixture of 6-bromo-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)nicotinic acid (50 mg, 0.068 mmol) and BOP (60 mg, 0.17 mmol) in THF (4.0 mL) were added N³,N³-dimethylpyridazine-3,6-diamine (9.4 mg, 0.068 mmol) and TEA (68 mg, 0.68 mmol). The mixture was stirred at 50° C. for 15 h.

The mixture was quenched with water (10 mL) and extracted with dichloromethane (3×15 mL). The combined organic phase was washed with brine, dried over Na₂SO₄, filtered and concentrated. The residue was purified by silica gel chromatography to provide the desired product (15 mg, 26% yield) as a yellow solid. MS(ESI) m/z=855.0 [M+H]⁺.

### Step 5. Synthesis of N-(6-(dimethylamino)pyridazin-3-yl)-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)-6-(methylamino)nicotinamide

To a mixture of 6-bromo-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)-amino)nicotinic acid (8 mg, 0.01 mmol) in NMP (1.5 mL) was added TEA (101 mg, 1.0 mmol) and propan-2-amine (32 mg, 0.55 mmol). The mixture was stirred at 150° C. for 1 h under microwave irradiation. The mixture was quenched with water (10 mL) and extracted with dichloromethane (3×15 mL). The combined organic phase was washed with brine, dried over Na₂SO₄, filtered and concentrated. The residue was purified by silica gel chromatography to provide the desired product (1.3 mg, 15% yield) as a light-yellow solid. MS (ESI) m/z=806.5[M+H]⁺.

### Example 712. N-(6-(dimethylamino)pyridazin-3-yl)-2-((2-(4-((6-((5-fluoro-4-(4-fluoro-1-isopropyl-2-methyl-1H-benzo[d]imidazol-6-yl)pyrimidin-2-yl)amino)pyridin-3-yl)methyl)piperazin-1-yl)-2-oxoethyl)amino)-6-(methylamino)nicotinamide (CPD-657)

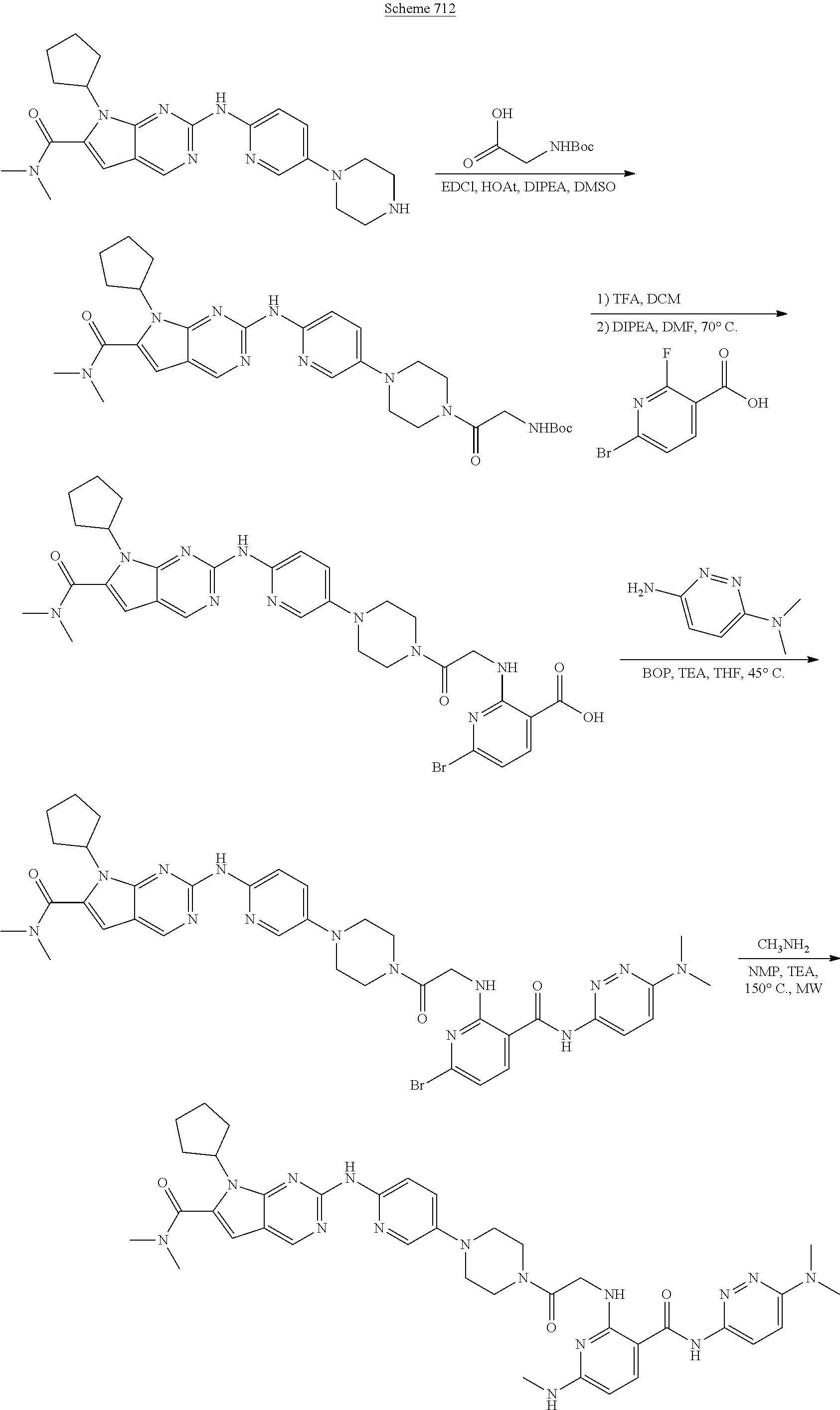

CPD-657 was synthesized following the standard procedures for preparing CPD-656 (2.3 mg, 3% yield over 5 steps) as a yellow solid. MS (ESI) m/z=762.5 [M+H]⁺.

### Example 713. N-(6-(dimethylamino)pyridazin-3-yl)-2-((2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethyl)amino)-6-(methylamino)nicotinamide (CPD-658)

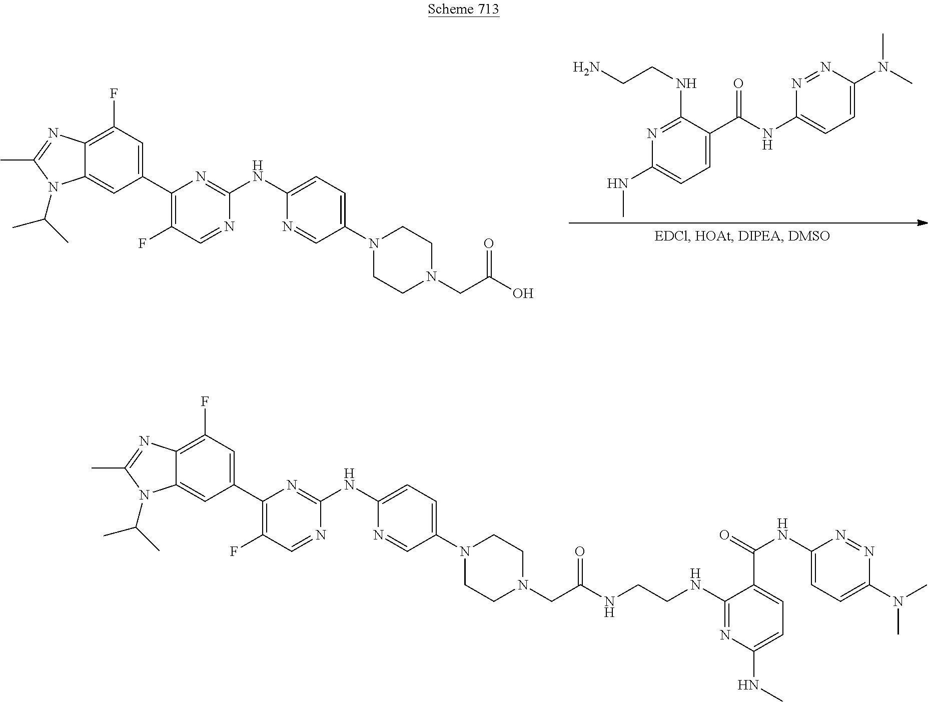

CPD-658 was synthesized following the standard procedure for preparing CPD-143 (2.3 mg, 18% yield) as a yellow solid. MS (ESI) m/z=835.2 [M+H]⁺.

### Example 714. N-(6-(dimethylamino)pyridazin-3-yl)-2-((3-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)propyl)amino)-6-(methylamino)nicotinamide (CPD-659)

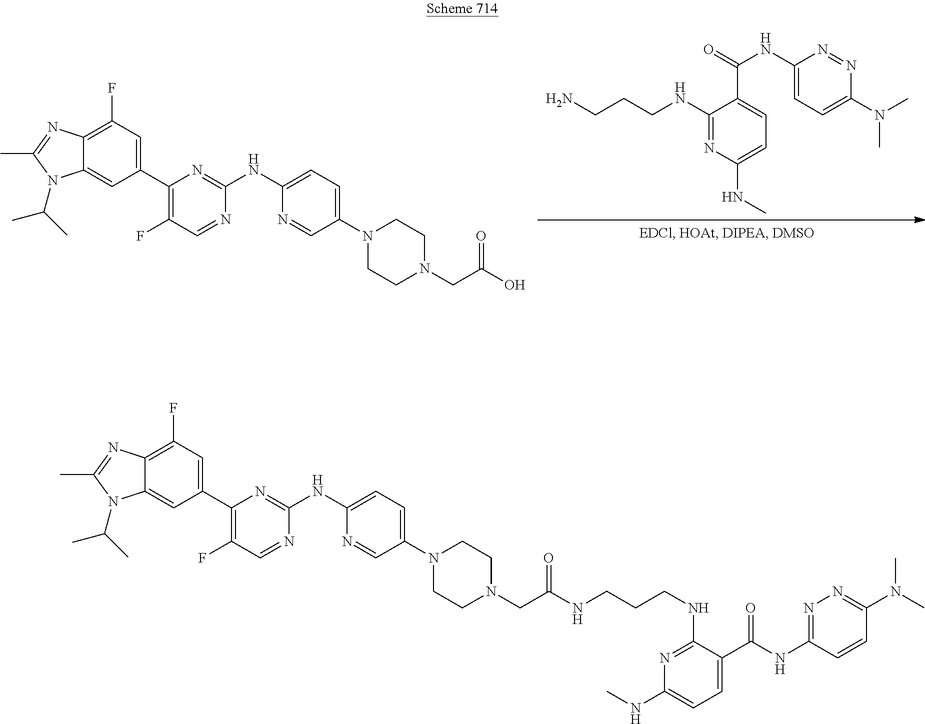

CPD-659 was synthesized following the standard procedure for preparing CPD-143 (2.6 mg, 20% yield) as a yellow solid. MS (ESI) nm/z=849.0 [M+H]⁺.

### Example 715. N-(6-(dimethylamino)pyridazin-3-yl)-2-((4-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)butyl)amino)-6-(methylamino)nicotinamide (CPD-660)

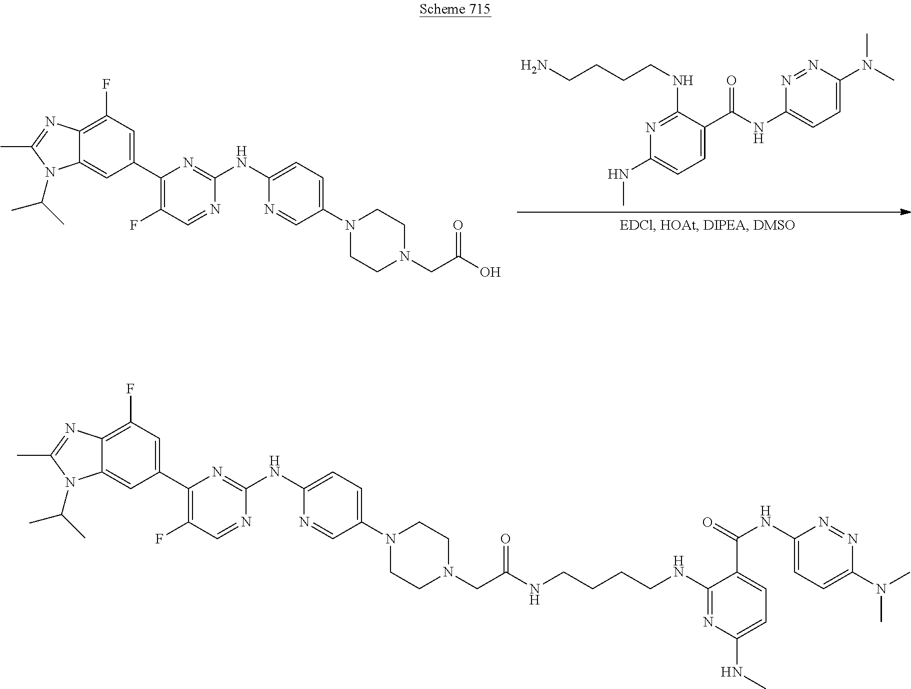

CPD-660 was synthesized following the standard procedure for preparing CPD-143 (2.9 mg, 22% yield) as a yellow solid. MS (ESI) m/z=863.2 [M+H]⁺.

### Example 716. N-(6-(dimethylamino)pyridazin-3-yl)-2-((5-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)pentyl)amino)-6-(methylamino)nicotinamide (CPD-661)

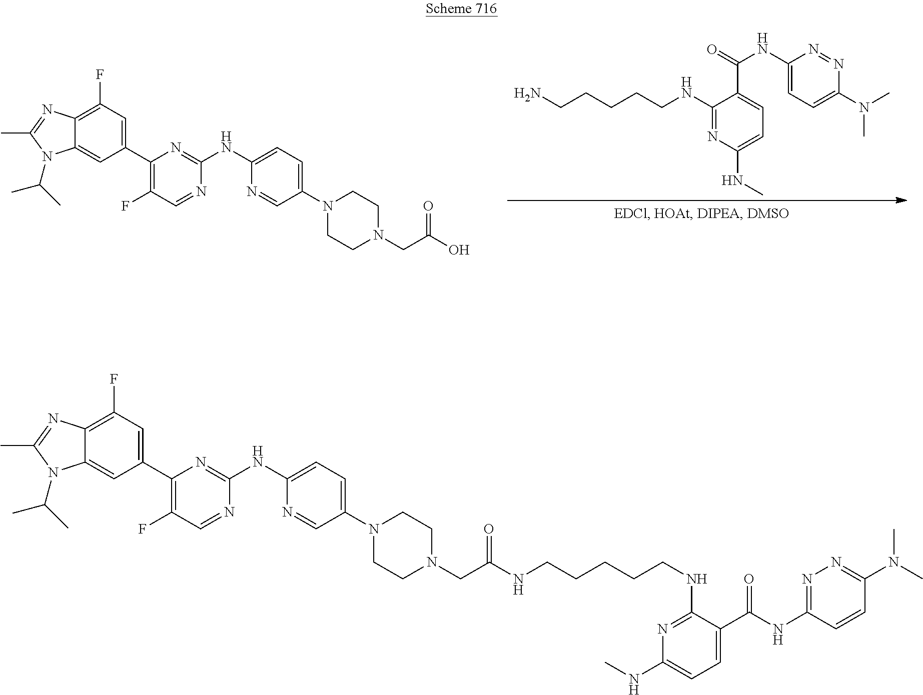

CPD-661 was synthesized following the standard procedure for preparing CPD-143 (4.3 mg, 32% yield) as a yellow solid. MS (ESI) m/z=877.2 [M+H]⁺.

### Example 717. N-(6-(dimethylamino)pyridazin-3-yl)-2-((6-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)hexyl)amino)-6-(methylamino)nicotinamide (CPD-662)

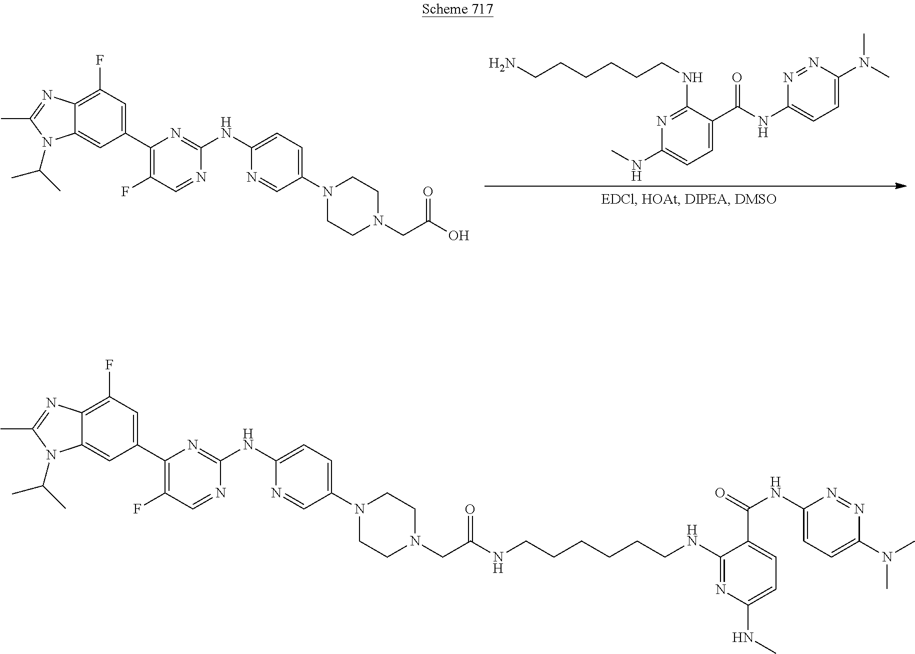

CPD-662 was synthesized following the standard procedure for preparing CPD-143 (2.9 mg, 22% yield) as a yellow solid. MS (ESI) m/z=891.2 [M+H]⁺.

### Example 718. N-(6-(dimethylamino)pyridazin-3-yl)-2-((7-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)heptyl)amino)-6-(methylamino)nicotinamide (CPD-663)

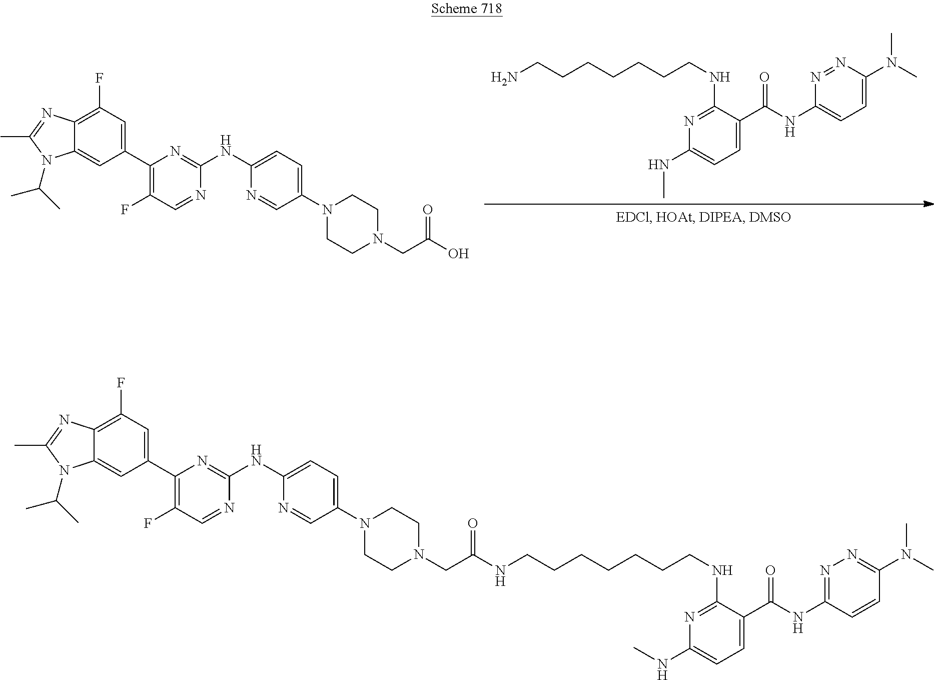

CPD-663 was synthesized following the standard procedure for preparing CPD-143 (4.1 mg, 30% yield) as a yellow solid. MS (ESI) m/z=905.3 [M+H]⁺.

### Example 719. N-(6-(dimethylamino)pyridazin-3-yl)-2-((8-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)octyl)amino)-6-(methylamino)nicotinamide (CPD-664)

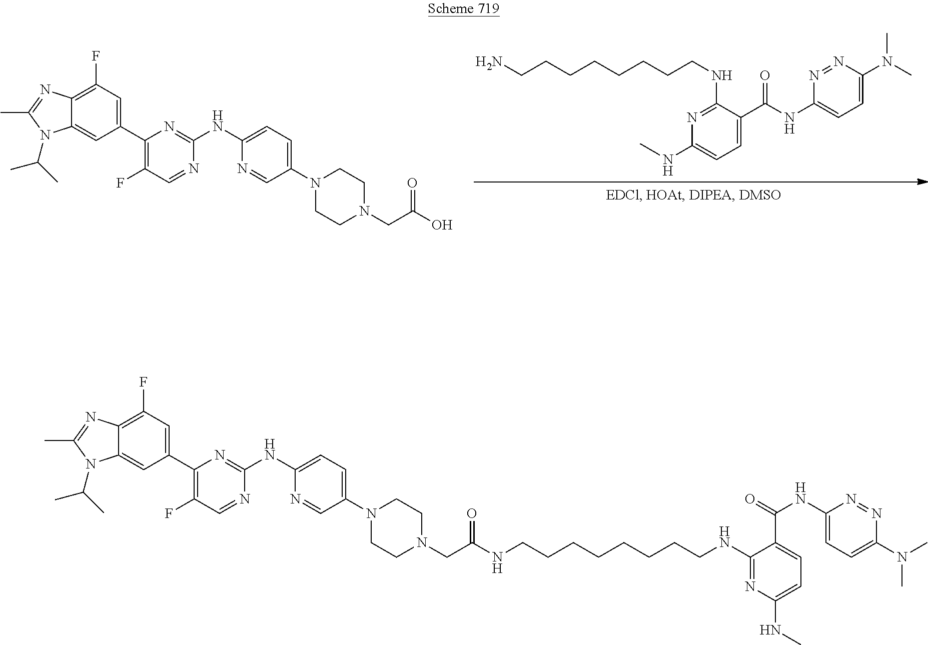

CPD-664 was synthesized following the standard procedure for preparing CPD-143 (4.8 mg, 35% yield) as a yellow solid. MS (ESI) m/z=919.3 [M+H]⁺.

### Example 720. N-(6-(dimethylamino)pyridazin-3-yl)-2-((2-(2-(2-(4-(6-((5-fluoro-4-(4-fluoro-1-isopropyl-2-methyl-1H-benzo[d]imidazol-6-yl)pyrimidin-2-yl)amino)pyridin-3-yl)piperazin-1-yl)acetamido)ethoxy)ethyl)amino)-6-(methylamino)nicotinamide (CPD-665)

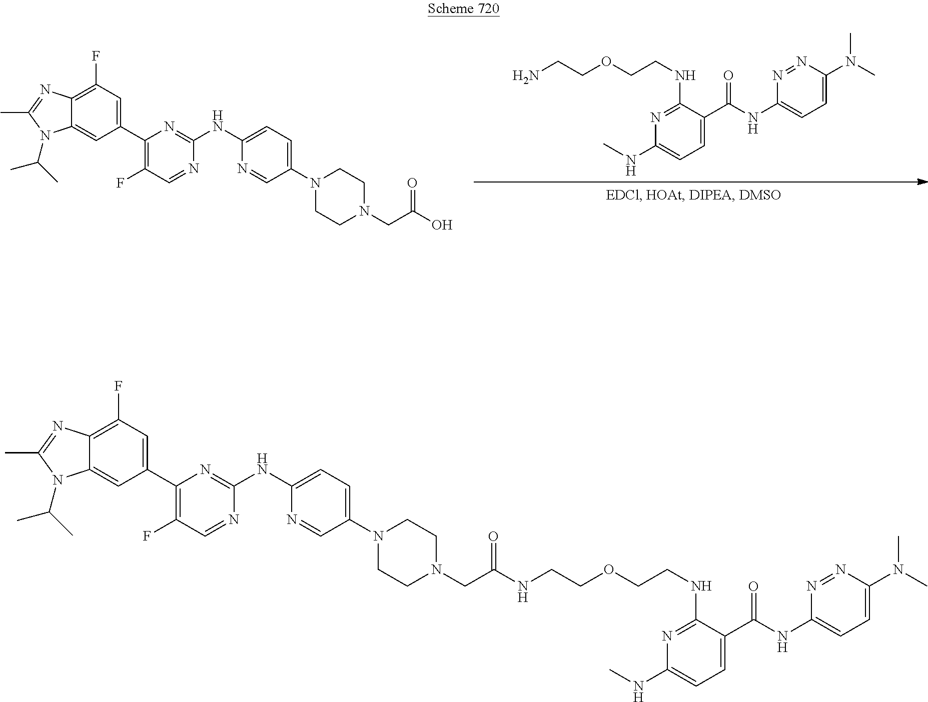

CPD-665 was synthesized following the standard procedure for preparing CPD-143 (4.1 mg, 35% yield) as a yellow solid. MS (ESI) m/z=880.0 [M+H]⁺.

### Example 721. 2-((3-((4-((8-cyclopentyl-7-oxo-7,8-dihydropyrido[2,3-d]pyrimidin-2-yl)amino)cyclohexyl)amino)-3-oxopropyl)amino)-N-(6-(dimethylamino)pyridazin-3-yl)-6-(methylamino)nicotinamide (CPD-666)

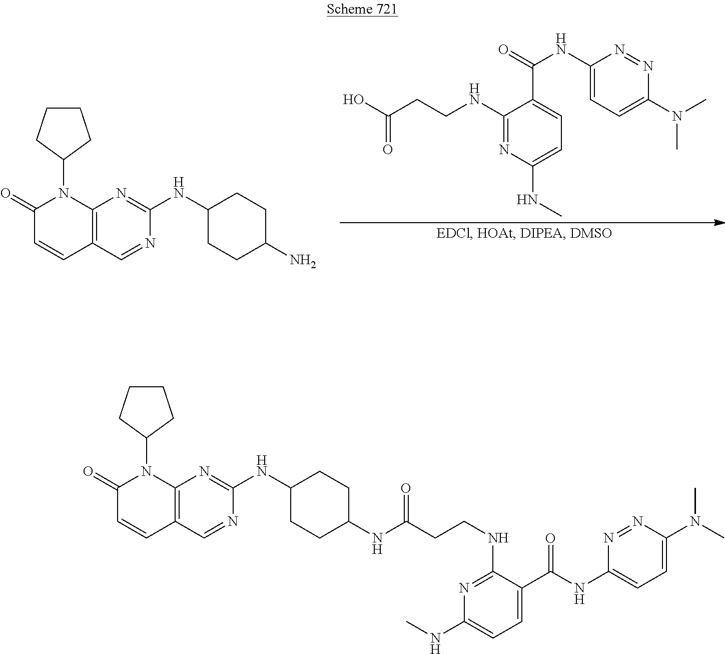

CPD-666 was synthesized following the standard procedure for preparing CPD-143 (1.6 mg, 10% yield) as a yellow solid. MS (ESI) m/z=669.2 [M+H]⁺.

### Example 722. 2-((4-((4-((8-cyclopentyl-7-oxo-7,8-dihydropyrido[2,3-d]pyrimidin-2-yl)amino)cyclohexyl)amino)-4-oxobutyl)amino)-N-(6-(dimethylamino)pyridazin-3-yl)-6-(methylamino)nicotinamide (CPD-667)

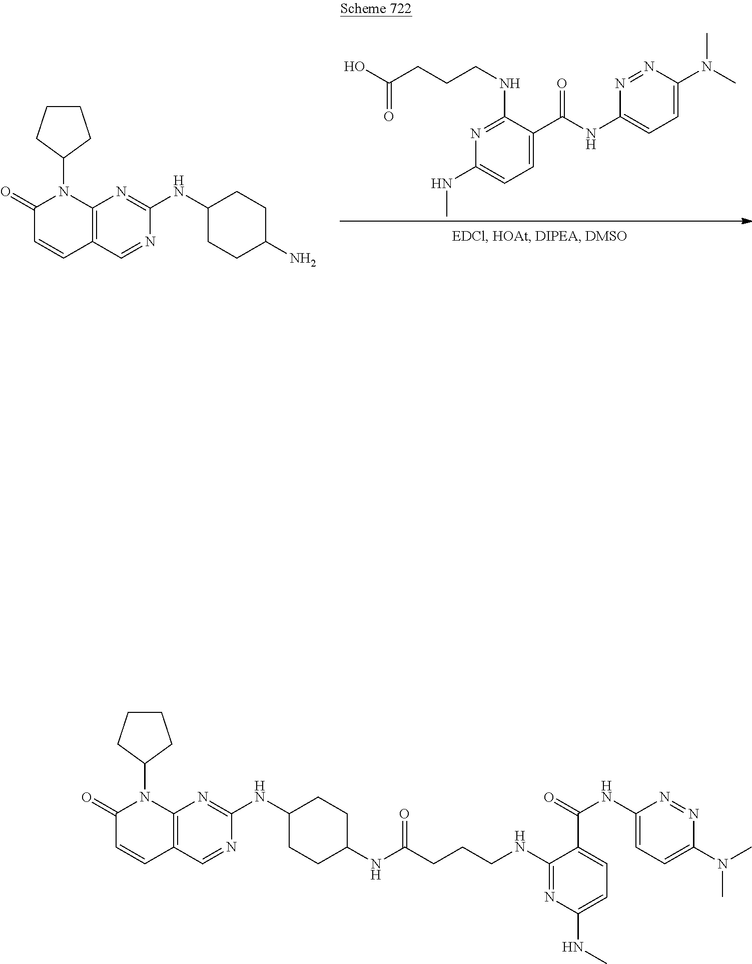 [SMILES: NC1CCC(Nc2ncc3ccc(=O)n(C4CCCC4)c3n2)CC1 ; CNc1ccc(C(=O)Nc2ccc(N(C)C)nn2)c(NCCCC(=O)O)n1 ; "EDCl, HOAt, DIPEA, DMSO" ; CNc1ccc(C(=O)Nc2ccc(N(C)C)nn2)c(NCCCC(=O)NC2CCC(Nc3ncc4ccc(=O)n(C5CCCC5)c4n3)CC2)n1]

CPD-667 was synthesized following the standard procedure for preparing CPD-143 (6.2 mg, 36% yield) as a yellow solid. MS (ESI) m/z=683.2 [M+H]⁺.

### Example 723. 2-((5-((4-((8-cyclopentyl-7-oxo-7,8-dihydropyrido[2,3-d]pyrimidin-2-yl)amino)cyclohexyl)amino)-5-oxopentyl)amino)-N-(6-(dimethylamino)pyridazin-3-yl)-6-(methylamino)nicotinamide (CPD-668)

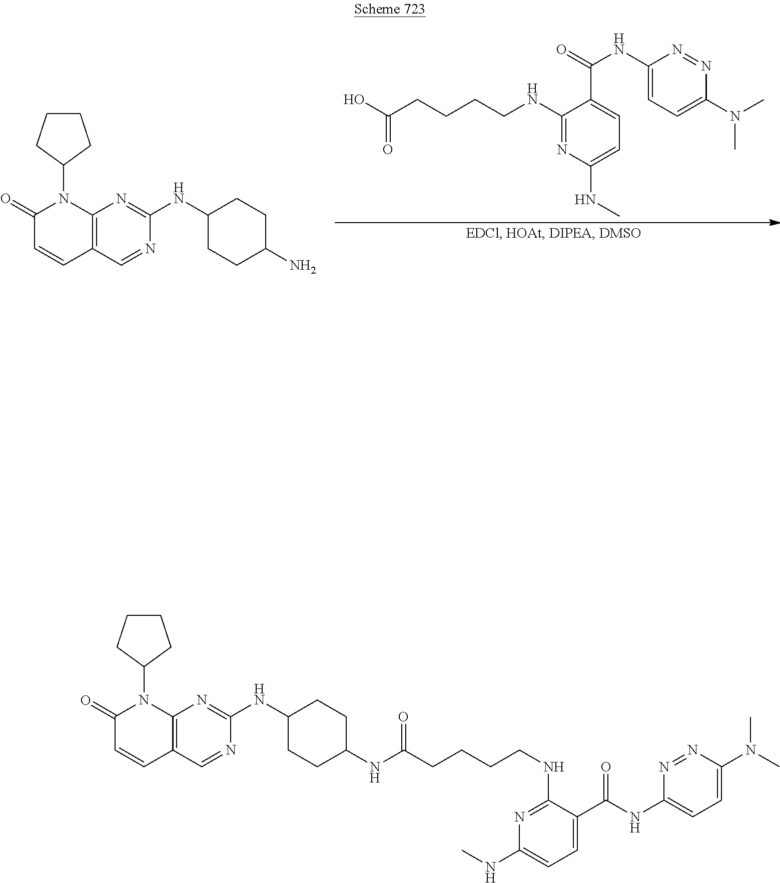 [SMILES: NC1CCC(Nc2ncc3ccc(=O)n(C4CCCC4)c3n2)CC1 ; CNc1ccc(C(=O)Nc2ccc(N(C)C)nn2)c(NCCCCC(=O)O)n1 ; "EDCl, HOAt, DIPEA, DMSO" ; CNc1ccc(C(=O)Nc2ccc(N(C)C)nn2)c(NCCCCC(=O)NC2CCC(Nc3ncc4ccc(=O)n(C5CCCC5)c4n3)CC2)n1]

CPD-668 was synthesized following the standard procedure for preparing CPD-143 (7.0 mg, 40% yield) as a yellow solid. MS (ESI) m/z=697.3 [M+H]⁺.

### Example 724. 2-((6-((4-((8-cyclopentyl-7-oxo-7,8-dihydropyrido[2,3-d]pyrimidin-2-yl)amino)cyclohexyl)amino)-6-oxohexyl)amino)-N-(6-(dimethylamino)pyridazin-3-yl)-6-(methylamino)nicotinamide (CPD-669)

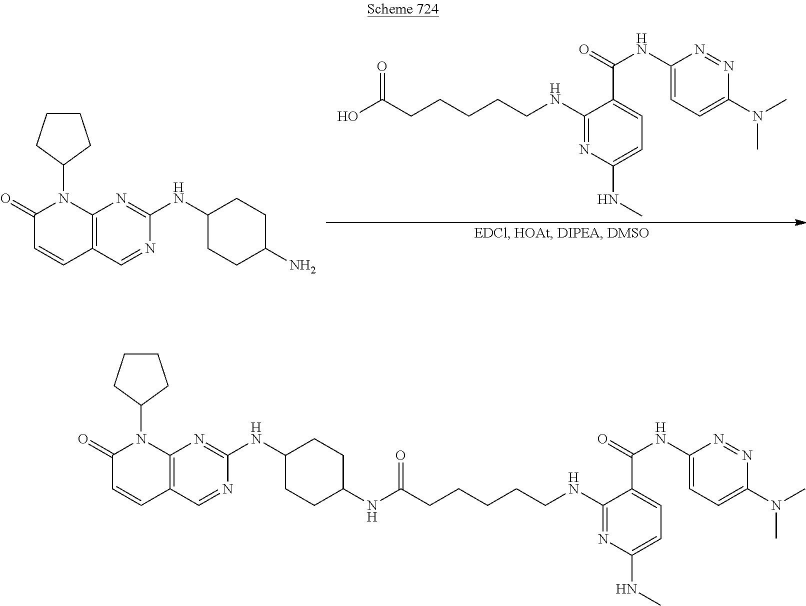 [SMILES: NC1CCC(Nc2ncc3ccc(=O)n(C4CCCC4)c3n2)CC1 ; CNc1ccc(C(=O)Nc2ccc(N(C)C)nn2)c(NCCCCCC(=O)O)n1 ; "EDCl, HOAt, DIPEA, DMSO" ; CNc1ccc(C(=O)Nc2ccc(N(C)C)nn2)c(NCCCCCC(=O)NC2CCC(Nc3ncc4ccc(=O)n(C5CCCC5)c4n3)CC2)n1]

CPD-669 was synthesized following the standard procedure for preparing CPD-143 (7.6 mg, 43% yield) as a yellow solid. MS (ESI) m/z=711.3 [M+H]⁺.

### Example 725. 2-((7-((4-((8-cyclopentyl-7-oxo-7,8-dihydropyrido[2,3-d]pyrimidin-2-yl)amino)cyclohexyl)amino)-7-oxoheptyl)amino)-N-(6-(dimethylamino)pyridazin-3-yl)-6-(methylamino)nicotinamide (CPD-670)

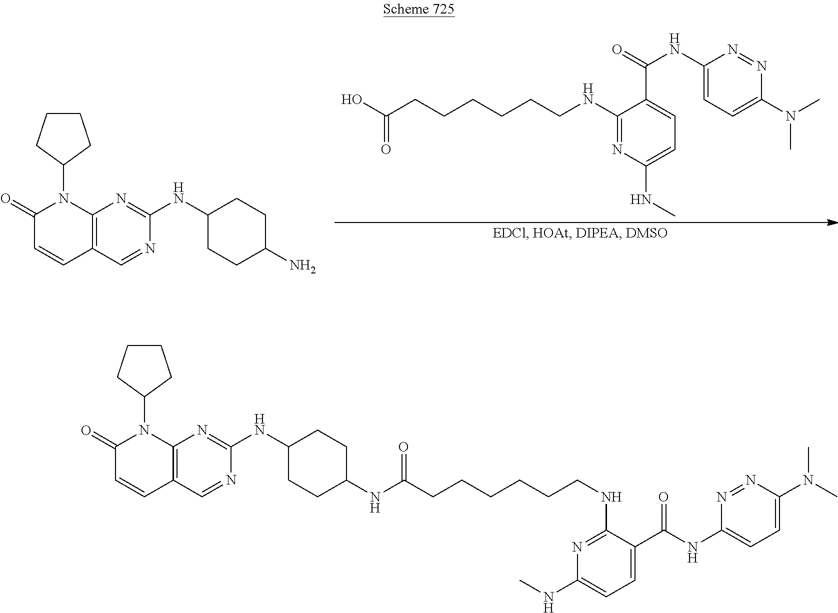 [SMILES: NC1CCC(Nc2ncc3ccc(=O)n(C4CCCC4)c3n2)CC1 ; CNc1ccc(C(=O)Nc2ccc(N(C)C)nn2)c(NCCCCCCC(=O)O)n1 ; "EDCl, HOAt, DIPEA, DMSO" ; CNc1ccc(C(=O)Nc2ccc(N(C)C)nn2)c(NCCCCCCC(=O)NC2CCC(Nc3ncc4ccc(=O)n(C5CCCC5)c4n3)CC2)n1]

CPD-670 was synthesized following the standard procedure for preparing CPD-143 (7.8 mg, 43% yield) as a yellow solid. MS (ESI) m/z=725.3 [M+H]⁺.

### Example 726. 2-((8-((4-((8-cyclopentyl-7-oxo-7,8-dihydropyrido[2,3-d]pyrimidin-2-yl)amino)cyclohexyl)amino)-8-oxooctyl)amino)-N-(6-(dimethylamino)pyridazin-3-yl)-6-(methylamino)nicotinamide (CPD-671)

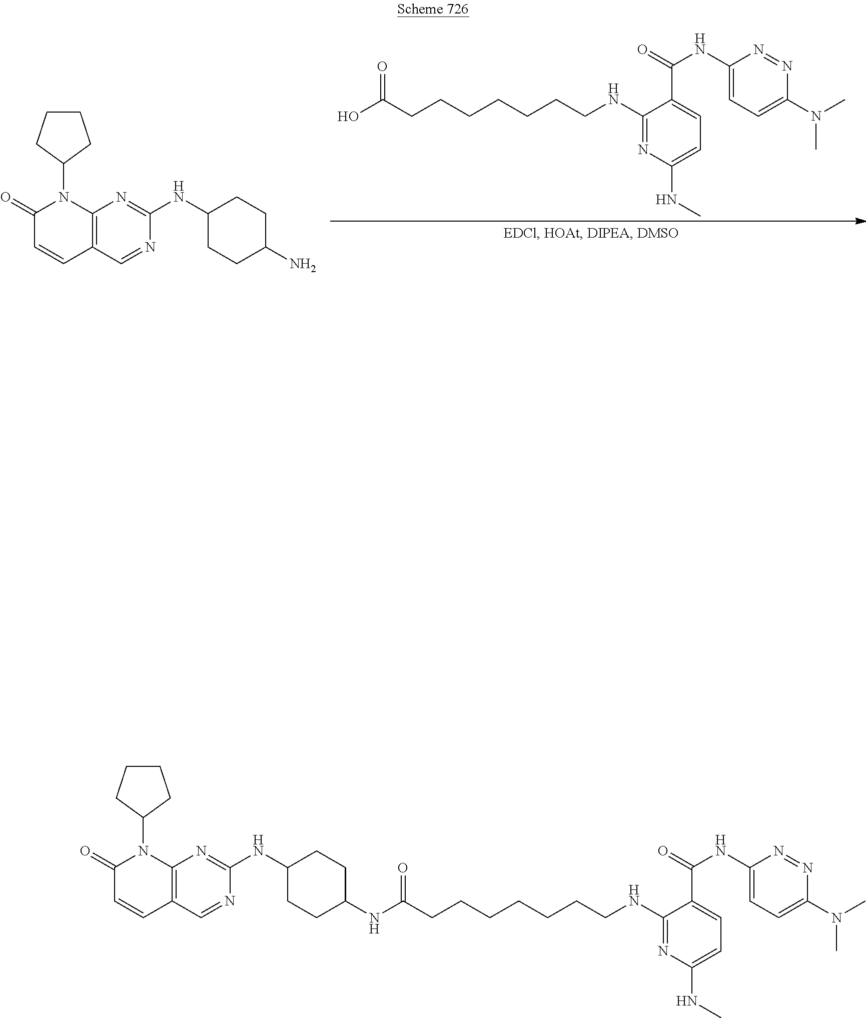 [SMILES: NC1CCC(Nc2ncc3ccc(=O)n(C4CCCC4)c3n2)CC1 ; CNc1ccc(C(=O)Nc2ccc(N(C)C)nn2)c(NCCCCCCCC(=O)O)n1 ; "EDCl, HOAt, DIPEA, DMSO" ; CNc1ccc(C(=O)Nc2ccc(N(C)C)nn2)c(NCCCCCCCC(=O)NC2CCC(Nc3ncc4ccc(=O)n(C5CCCC5)c4n3)CC2)n1]

CPD-671 was synthesized following the standard procedure for preparing CPD-143 (7.0 mg, 38% yield) as a yellow solid. MS (ESI) m/z=739.3 [M+H]⁺.

### Example 727. 2-((2-(3-((4-((8-cyclopentyl-7-oxo-7,8-dihydropyrido[2,3-d]pyrimidin-2-yl)amino)cyclohexyl)amino)-3-oxopropoxy)ethyl)amino)-N-(6-(dimethylamino)pyridazin-3-yl)-6-(methylamino)nicotinamide (CPD-672)

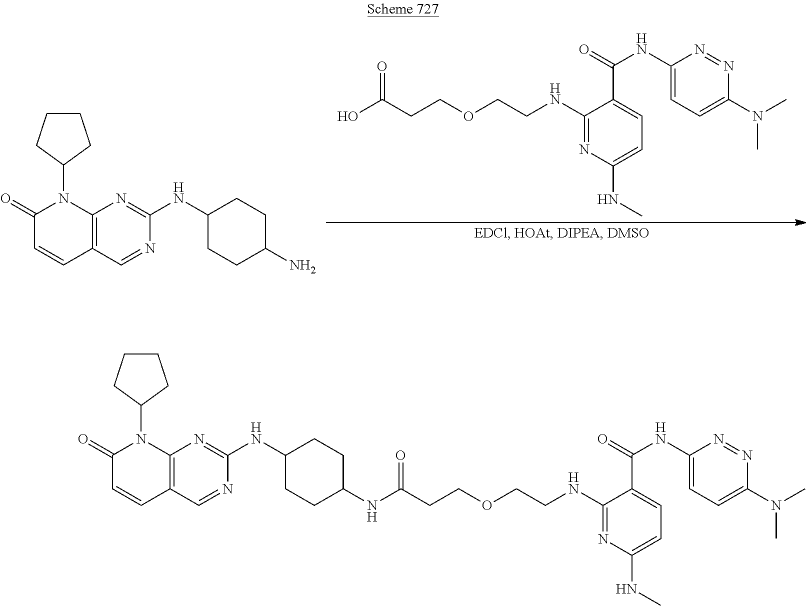 [SMILES: NC1CCC(Nc2ncc3ccc(=O)n(C4CCCC4)c3n2)CC1 ; CNc1ccc(C(=O)Nc2ccc(N(C)C)nn2)c(NCCOCCC(=O)O)n1 ; "EDCl, HOAt, DIPEA, DMSO" ; CNc1ccc(C(=O)Nc2ccc(N(C)C)nn2)c(NCCOCCC(=O)NC2CCC(Nc3ncc4ccc(=O)n(C5CCCC5)c4n3)CC2)n1]

CPD-672 was synthesized following the standard procedure for preparing CPD-143 (8.1 mg, 46% yield) as a yellow solid. MS (ESI) m/z=713.3 [M+H]⁺.

### Example 728. 2-((3-((2-((8-cyclopentyl-7-oxo-7,8-dihydropyrido[2,3-d]pyrimidin-2-yl)amino)spiro[3.5]nonan-7-yl)amino)-3-oxopropyl)amino)-N-(6-(dimethylamino)pyridazin-3-yl)-6-(methylamino)nicotinamide (CPD-673)

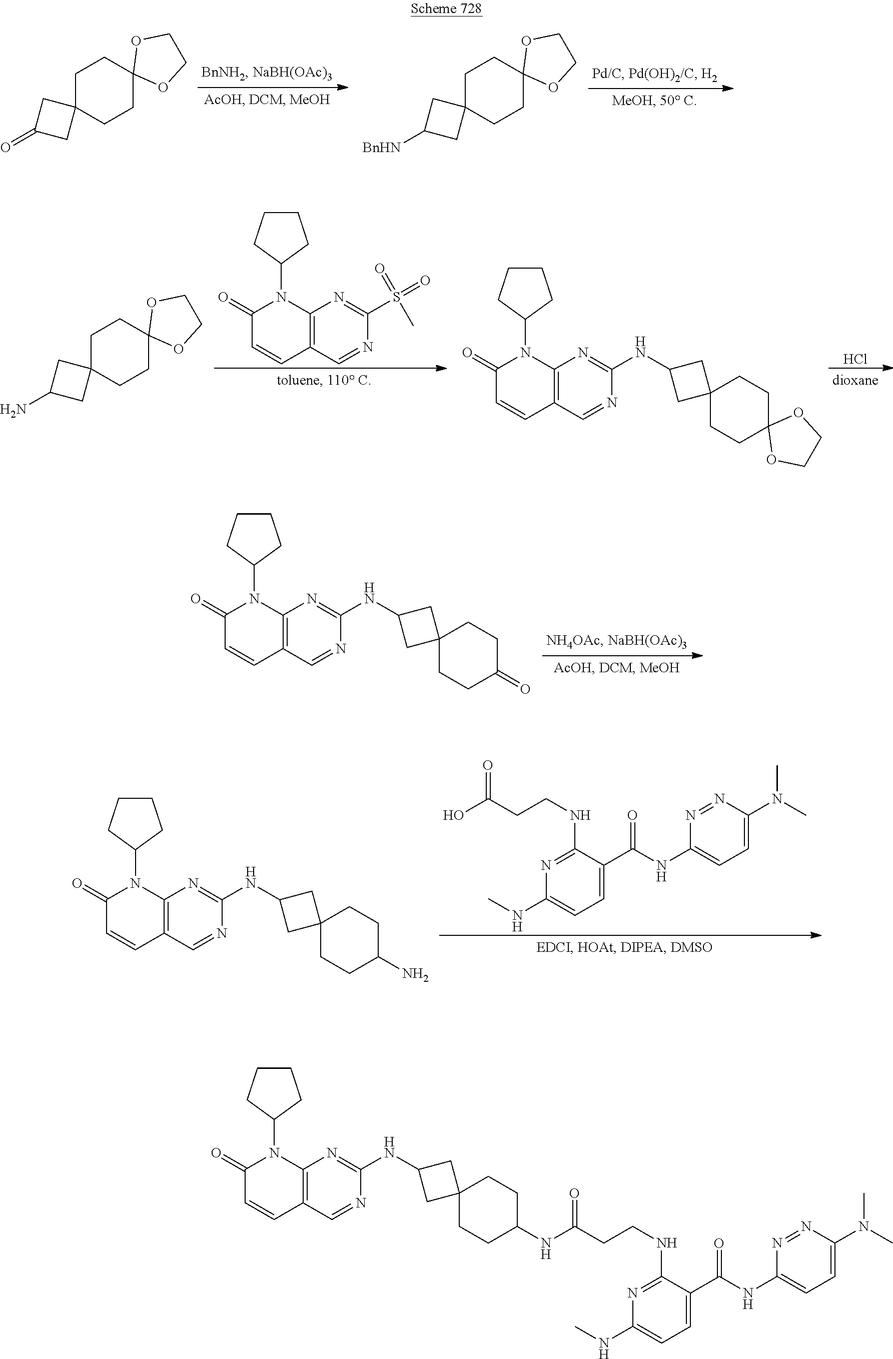

### Step 1. Synthesis of N-benzyl-8,11-dioxadispiro[3.2.4⁷.2⁴]tridecan-2-amine

A mixture of 8,11-dioxadispiro[3.2.4⁷.2⁴]tridecan-2-one (500 mg, 2.6 mmol) and phenylmethanamine (819 mg, 7.6 mmol) in AcOH (0.5 mL), dichloromethane (10 mL) and MeOH (2 mL) was stirred at rt for 2 h, followed by adding NaBH(OAc)₃ (1.3 g, 6.4 mmol) in portions. The mixture was stirred at rt for 24 h. The reaction mixture was diluted with aq. NaHCO₃ solution (15 mL, 5 N) and extracted with dichloromethane (3×25 mL). The combined organic phase was washed with brine, dried over Na₂SO₄, filtered, and concentrated to provide the crude compound (550 mg, 75% yield) as a white solid. This compound was used directly in the next step without further purification. MS (ESI) m/z=288.7 [M+H]⁺.

### Step 2. Synthesis of 8,11-dioxadispiro[3.2.4⁷.2⁴]tridecan-2-amine

A mixture of N-benzyl-8,11-dioxadispiro[3.2.4⁷.2⁴]tridecan-2-amine (550 mg, 1.9 mmol), 10% Pd/C (55 mg) and 10% Pd(OH)₂/C (55 mg) in MeOH (10 mL) was degassed and stirred under at 50° C. for 12 h under hydrogen balloon. The mixture was filtered and the cake was washed with dichloromethane (20 mL) and MeOH (20 mL). The filtrate was concentrated to provide the crude compound (350 mg, 93% yield) as a white solid. This compound was used directly in the next step without further purification. MS (ESI) m/z=198.4 [M+H]⁺.

### Step 3. Synthesis of 2-((8,11-dioxadispiro[3.2.4⁷0.24]tridecan-2-yl)amino)-8-cyclopentyl pyrido[2,3-d]pyrimidin-7(8H)-one

A mixture of 8,11-dioxadispiro[3.2.4⁷.2⁴]tridecan-2-amine (350 mg crude, 1.8 mmol) and 8-cyclopentyl-2-(methylsulfonyl)pyrido[2,3-d]pyrimidin-7(8H)-one (520 mg, 1.8 mmol) in toluene (10 mL) was stirred at 110° C. for 4 h. After cooling down to rt, the mixture was concentrated and purified by silica gel chromatography to provide the title compound (300 mg, 41% yield) as a white solid. MS (ESI) m/z=411.8 [M+H]⁺.

### Step 4. Synthesis of 8-cyclopentyl-2-((7-oxospiro[3.5]nonan-2-yl)amino)pyrido[2,3-d]pyrimidin-7(8H)-one

A solution of 2-((8,11-dioxadispiro[3.2.4⁷.2⁴]tridecan-2-yl)amino)-8-cyclopentylpyrido[2,3-d]pyrimidin-7(8H)-one (200 mg, 0.49 mmol) in HCl solution (6 mL, 4 M in dioxane) was stirred at rt for 12 h. The mixture was concentrated to give the crude compound (150 mg, 84% yield) as a white solid. This compound was used directly in the next step without further purification. MS (ESI) m/z=367.6 [M+H]⁺.

### Step 5. Synthesis of 2-((7-aminospiro[3.5]nonan-2-yl)amino)-8-cyclopentylpyrido[2,3-d]pyrimidin-7(8H)-one

A mixture of 8-cyclopentyl-2-((7-oxospiro[3.5]nonan-2-yl)amino)pyrido[2,3-d]pyrimidin-7(8H)-one (150 mg, 0.41 mmol) and NH₄OAc (158 mg, 2.1 mmol) in AcOH (0.5 mL), dichloromethane (6 mL) and MeOH (2 mL) was stirred at rt for 1 h, followed by adding NaBH(OAc)₃ (259 mg, 1.2 mmol). The mixture was stirred at rt for 12 h. The reaction mixture was diluted with aq. NaHCO₃ solution (8 mL, 5 N) and extracted with dichloromethane (3×20 mL). The combined organic phase was washed with brine, dried over Na₂SO₄, filtered and concentrated. The residue was purified by silica gel chromatography to provide the title compound (35 mg, 23% yield) as a white solid. MS (ESI) m/z=368.7 [M+H]⁺.

### Step 6. Synthesis of 2-((3-((2-((8-cyclopentyl-7-oxo-7,8-dihydropyrido[2,3-d]pyrimidin-2-yl)amino)spiro[3.5]nonan-7-yl)amino)-3-oxopropyl)amino)-N-(6-(dimethyl amino)pyridazin-3-yl)-6-(methylamino)nicotinamide

To a mixture of 3-((3-((6-(dimethylamino)pyridazin-3-yl)carbamoyl)-6-(methylamino)pyridin-2-yl)amino)propanoic acid (3.2 mg, 9.0 μmol), EDCI (3.9 mg, 20.4 mol) and HOAt (1.1 mg, 8.2 μmol) in DMSO (0.5 mL) were added DIPEA (5.3 mg, 40.8 μmol) and 2-((7-aminospiro[3.5]nonan-2-yl)amino)-8-cyclopentylpyrido[2,3-d]pyrimidin-7(8H)-one (3.0 mg, 8.2 μmol). The mixture was stirred at rt for 12 h. The mixture was purified by prep-HPLC to provide the title compound (0.70 mg, 11% yield) as a yellow solid. MS (ESI) m/z=709.3 [M+H]⁺.

### Example 729. 2-((4-((2-((8-cyclopentyl-7-oxo-7,8-dihydropyrido[2,3-d]pyrimidin-2-yl)amino)spiro[3.5]nonan-7-yl)amino)-4-oxobutyl)amino)-N-(6-(dimethylamino)pyridazin-3-yl)-6-(methylamino)nicotinamide (CPD-674)

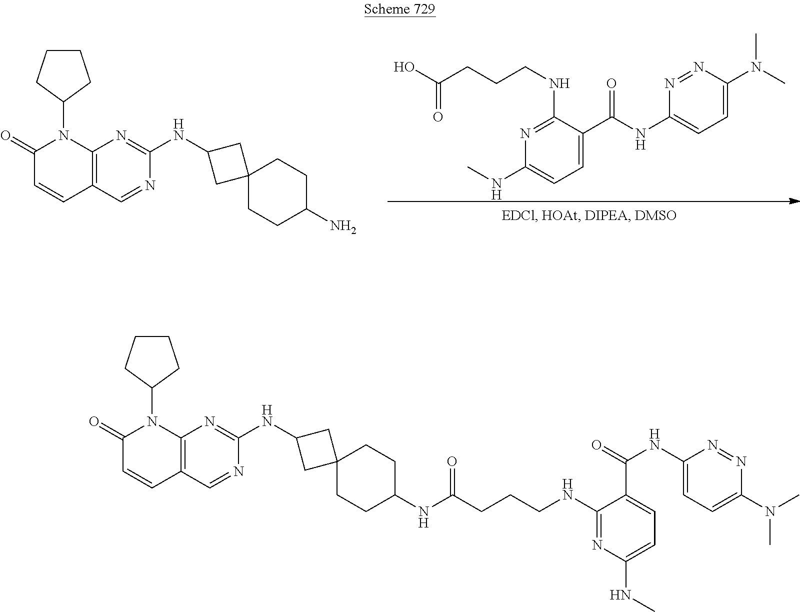 [SMILES: NC1CCC2(CC1)CC(Nc1ncc3ccc(=O)n(C4CCCC4)c3n1)C2 ; CNc1ccc(C(=O)Nc2ccc(N(C)C)nn2)c(NCCCC(=O)O)n1 ; "EDCl, HOAt, DIPEA, DMSO" ; CNc1ccc(C(=O)Nc2ccc(N(C)C)nn2)c(NCCCC(=O)NC2CCC3(CC2)CC(Nc2ncc4ccc(=O)n(C5CCCC5)c4n2)C3)n1]

CPD-674 was synthesized following the standard procedure for preparing CPD-143 (0.6 mg, 10% yield) as a yellow solid. MS (ESI) m/z=723.4 [M+H]⁺.

### Example 730. 2-((5-((2-((8-cyclopentyl-7-oxo-7,8-dihydropyrido[2,3-d]pyrimidin-2-yl)amino)spiro[3.5]nonan-7-yl)amino)-5-oxopentyl)amino)-N-(6-(dimethylamino)pyridazin-3-yl)-6-(methylamino)nicotinamide (CPD-675)

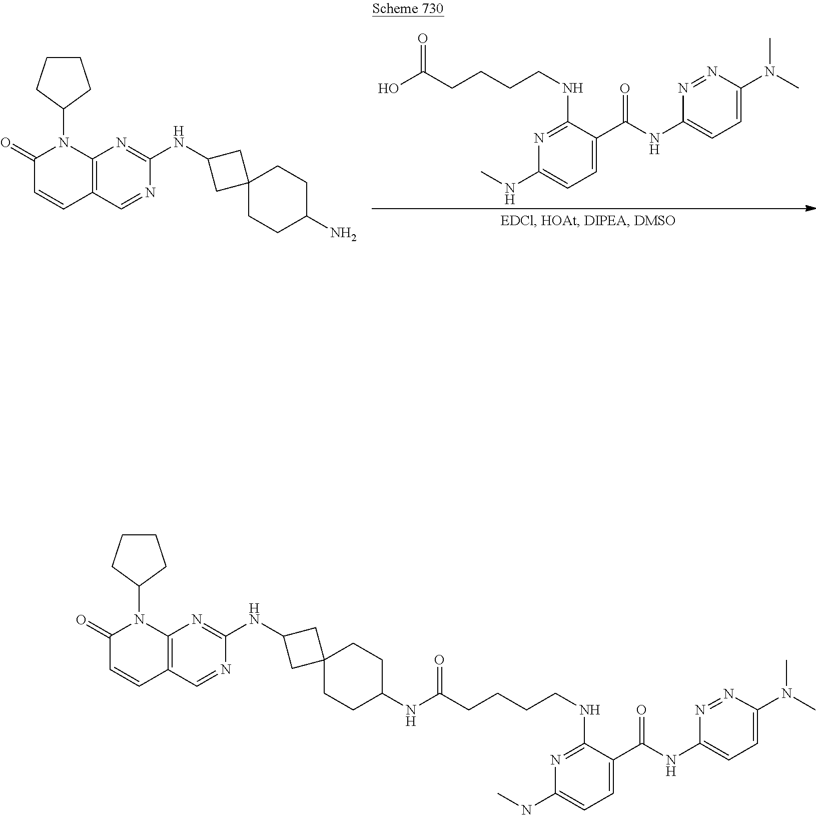

CPD-675 was synthesized following the standard procedure for preparing CPD-143 (0.6 mg, 10% yield) as a yellow solid. MS (ESI) m/z=727.4 [M+H]⁺.

### Example 731. 2-((6-((2-((8-cyclopentyl-7-oxo-7,8-dihydropyrido[2,3-d]pyrimidin-2-yl)amino)spiro[3.5]nonan-7-yl)amino)-6-oxohexyl)amino)-N-(6-(dimethylamino)pyridazin-3-yl)-6-(methylamino)nicotinamide (CPD-676)

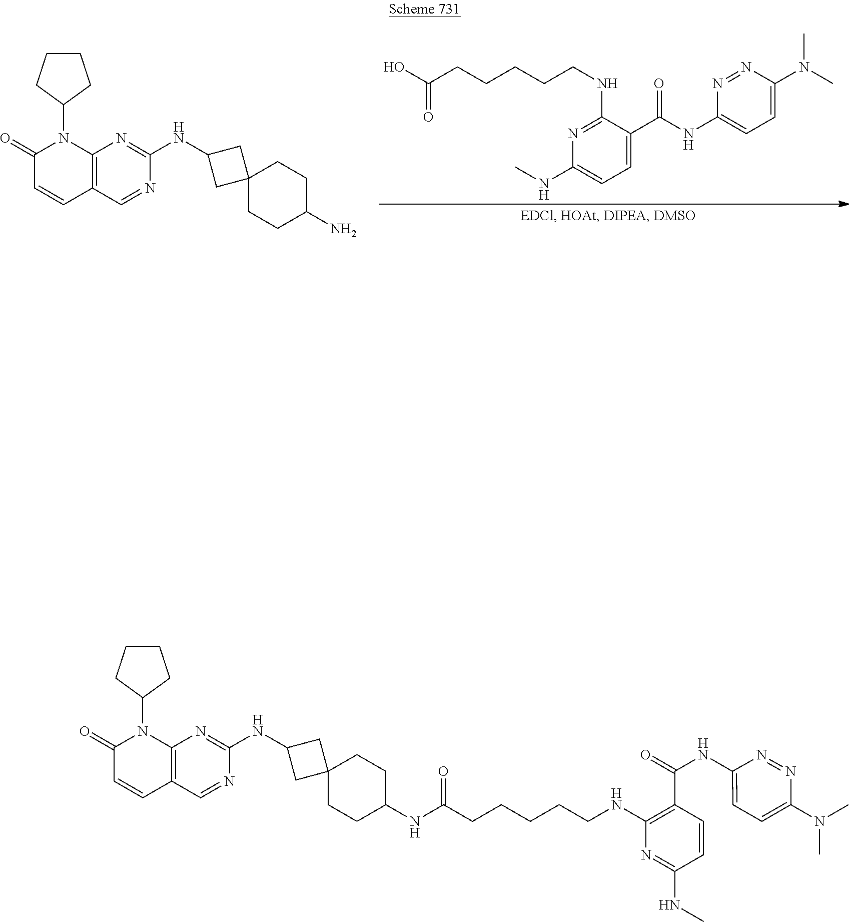

CPD-676 was synthesized following the standard procedure for preparing CPD-143 (0.51 mg, 8% yield) as a yellow solid. MS (ESI) m/z=751.4 [M+H]⁺.

### Example 732. 2-((7-((2-((8-cyclopentyl-7-oxo-7,8-dihydropyrido[2,3-d]pyrimidin-2-yl)amino)spiro[3.5]nonan-7-yl)amino)-7-oxoheptyl)amino)-N-(6-(dimethylamino)pyridazin-3-yl)-6-(methylamino)nicotinamide (CPD-677)

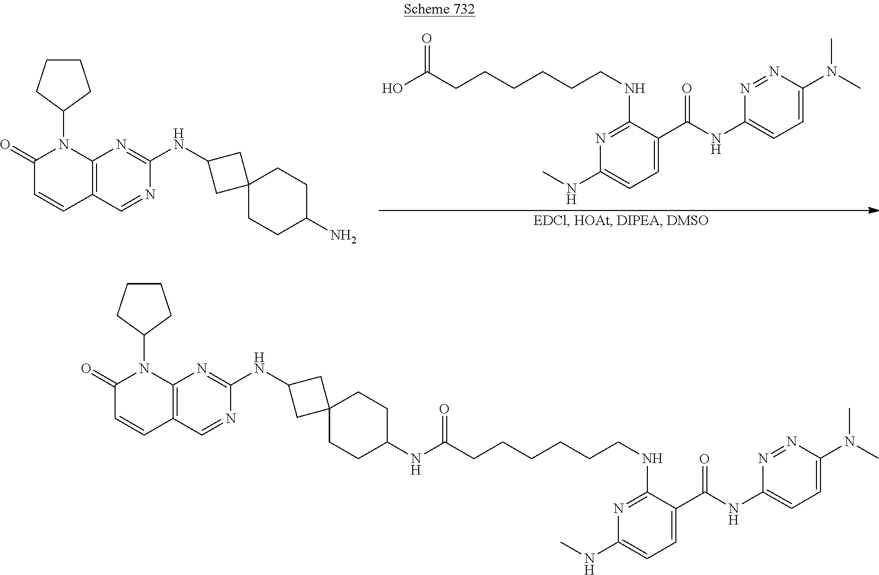

CPD-677 was synthesized following the standard procedure for preparing CPD-143 (0.71 mg, 11% yield) as a yellow solid. MS (ESI) m/z=765.5 [M+H]⁺.

### Example 733. 2-((8-((2-((8-cyclopentyl-7-oxo-7,8-dihydropyrido[2,3-d]pyrimidin-2-yl)amino)spiro[3.5]nonan-7-yl)amino)-8-oxooctyl)amino)-N-(6-(dimethylamino)pyridazin-3-yl)-6-(methylamino)nicotinamide (CPD-678)

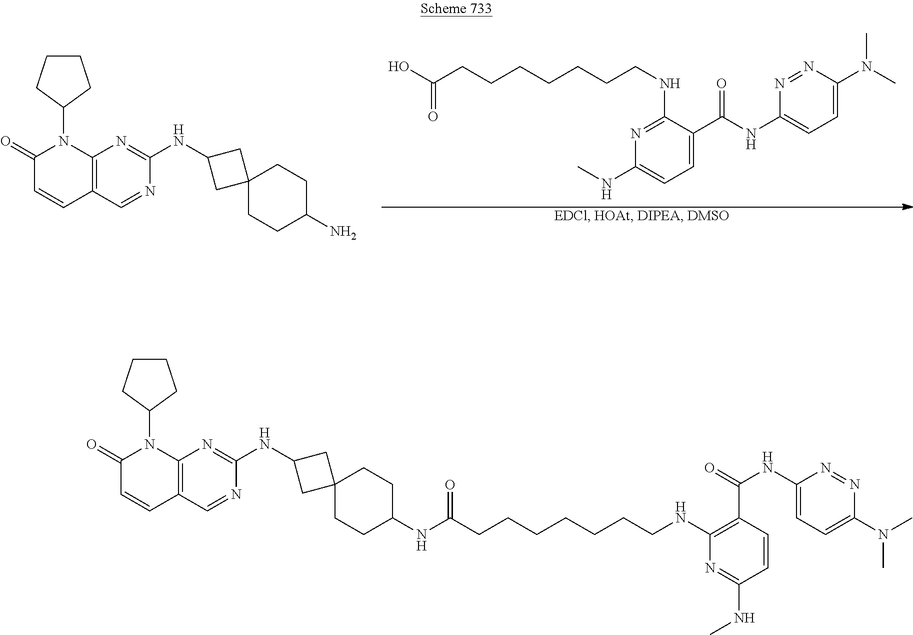

CPD-678 was synthesized following the standard procedure for preparing CPD-143 (0.71 mg, 11% yield) as a yellow solid. MS (ESI) m/z=765.5 [M+H]⁺.

### Example 734. 2-((2-(3-((2-((8-cyclopentyl-7-oxo-7,8-dihydropyrido[2,3-d]pyrimidin-2-yl)amino)spiro[3.5]nonan-7-yl)amino)-3-oxopropoxy)ethyl)amino)-N-(6-(dimethylamino)pyridazin-3-yl)-6-(methylamino)nicotinamide (CPD-679)

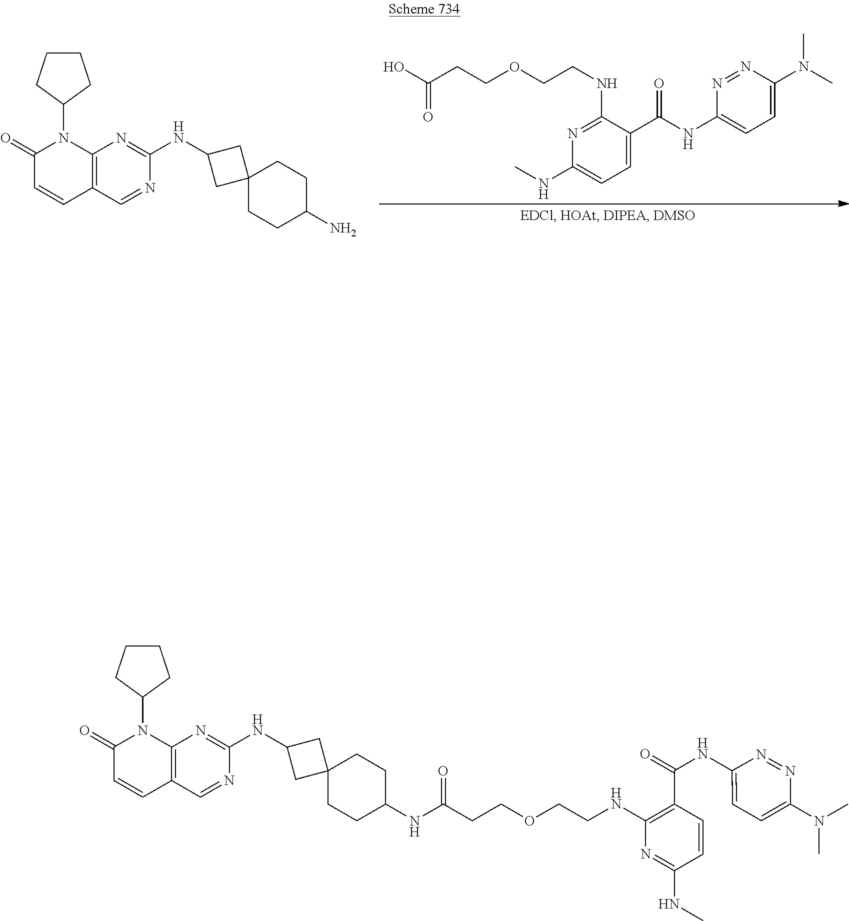

CPD-679 was synthesized following the standard procedure for preparing CPD-143 (0.9 mg, 15% yield) as a yellow solid. MS (ESI) m/z=753.3 [M+H]⁺.

### Example 735. CPD-10 Concentration-Dependently Reduced CCND1, CCND3, CDK4, FoxM1, cyclin A2, and p-Rb Protein Levels (FIG. 1A-1B)

Calu-1 cells were treated with heterobifunctional compound CPD-10, or CDK4/6 inhibitor palbociclib at indicated concentrations for 4 hours (FIG. 1A). MCF7 cells were deprived of serum for 2 hours followed by treatment with CPD-10, or palbociclib at indicated concentrations for 24 hours (FIG. 1B). CPD-10 reduced CCND1, CCND3, and CDK4 protein levels in a concentration-dependent manner in both cell lines. In contrast, palbociclib did not significantly change the levels of these proteins in Calu-1 cells, or even accumulated CCND1 protein levels in MCF7 cells. In addition, CPD-10 also inhibited downstream FoxM1 and cyclin A2 protein levels, and Rb phosphorylation in a concentration-dependent manner.

### Example 736. Heterobifunctional Compounds Reduced CCND1 and CDK4 Protein Levels (Table 6)

The cellular protein degradation results of selected heterobifunctional compounds are set forth in Table 6. Calu-(cells were treated with heterobifunctional compounds at 40 nM or 200 nM for 16 hours A: protein percentage degradation ≥800%; B: protein percentage degradation <80%, and ≥50%; C: protein percentage degradation <50%, and ≥30%; D; protein percentage degradation <300%; -, data not available.

TABLE 6
CCND1 and CDK4 percentage degradation in Calu-1 Cell.

| | CyclinD1 | CyclinD1 | CDK4 | CDK4 |
|---|---|---|---|---|
| | Degradation | Degradation | Degradation | Degradation |
| Cpd. No. | (40 nM) | (200 nM) | (40 nM) | (200 nM) |
| CPD-1 | D | C | D | C |
| CPD-2 | A | A | B | A |
| CPD-3 | A | A | B | B |
| CPD-4 | A | A | B | — |
| CPD-5 | A | A | B | — |
| CPD-6 | A | A | B | — |
| CPD-7 | A | A | B | — |
| CPD-8 | A | A | B | A |
| CPD-9 | A | A | B | A |
| CPD-10 | A | A | B | A |
| CPD-11 | A | A | B | A |
| CPD-12 | — | — | — | — |
| CPD-13 | — | — | — | — |
| CPD-14 | A | A | B | B |
| CPD-15 | D | D | D | D |
| CPD-16 | A | A | B | B |
| CPD-17 | D | D | D | C |
| CPD-18 | A | A | B | B |
| CPD-19 | D | — | D | — |
| CPD-20 | D | — | D | — |
| CPD-21 | B | — | B | — |
| CPD-22 | A | A | A | A |
| CPD-23 | D | D | D | D |
| CPD-24 | A | A | B | A |
| CPD-25 | D | A | D | B |
| CPD-26 | A | A | B | B |
| CPD-27 | A | A | A | A |
| CPD-28 | A | A | B | B |
| CPD-29 | A | A | B | A |
| CPD-30 | A | A | B | A |
| CPD-31 | D | A | D | B |
| CPD-32 | D | D | D | D |
| CPD-33 | A | A | B | A |
| CPD-34 | A | A | A | A |
| CPD-35 | A | A | B | B |
| CPD-36 | D | A | C | B |
| CPD-37 | A | A | B | B |
| CPD-38 | A | A | B | A |
| CPD-39 | A | A | B | B |
| CPD-40 | A | A | B | B |
| CPD-41 | A | A | C | B |
| CPD-42 | D | D | D | D |
| CPD-43 | A | A | D | B |
| CPD-44 | D | A | D | C |
| CPD-45 | D | A | D | D |
| CPD-46 | D | D | D | D |
| CPD-47 | D | A | D | B |
| CPD-48 | D | D | D | D |
| CPD-49 | B | A | B | B |
| CPD-50 | A | A | B | B |
| CPD-51 | A | A | B | B |
| CPD-52 | D | A | D | B |
| CPD-53 | A | A | B | B |
| CPD-54 | A | A | B | B |
| CPD-55 | D | D | D | D |
| CPD-56 | A | A | B | B |
| CPD-57 | A | A | C | A |
| CPD-58 | D | A | C | B |
| CPD-59 | A | A | B | B |
| CPD-60 | B | A | B | B |
| CPD-61 | D | D | D | D |
| CPD-62 | A | A | A | A |
| CPD-63 | A | A | A | A |
| CPD-64 | D | D | D | D |
| CPD-65 | D | D | D | D |
| CPD-66 | A | A | B | B |
| CPD-67 | D | D | D | D |
| CPD-68 | A | A | C | B |
| CPD-69 | D | A | C | B |
| CPD-70 | A | A | B | B |
| CPD-71 | D | D | D | D |
| CPD-72 | B | A | B | A |
| CPD-73 | A | A | B | B |
| CPD-74 | D | D | D | D |
| CPD-75 | A | A | C | B |
| CPD-76 | D | C | D | C |
| CPD-77 | D | D | D | D |
| CPD-78 | A | A | B | B |
| CPD-79 | A | A | B | B |
| CPD-80 | A | A | B | B |
| CPD-81 | A | A | C | C |
| CPD-82 | A | A | C | C |
| CPD-83 | D | C | D | C |
| CPD-84 | D | D | D | D |
| CPD-85 | D | A | D | B |
| CPD-86 | D | D | D | D |
| CPD-87 | D | D | D | D |
| CPD-88 | B | A | C | B |
| CPD-89 | B | A | B | B |
| CPD-90 | C | B | D | B |
| CPD-91 | D | B | D | C |
| CPD-92 | D | D | D | D |
| CPD-93 | B | A | C | B |
| CPD-94 | D | C | D | C |
| CPD-95 | D | D | D | D |
| CPD-96 | D | D | D | D |
| CPD-97 | B | A | B | A |
| CPD-98 | D | D | C | B |
| CPD-99 | D | A | C | B |
| CPD-100 | D | A | D | B |
| CPD-101 | A | A | B | A |
| CPD-102 | B | A | B | B |
| CPD-103 | A | A | A | A |
| CPD-104 | D | A | D | D |
| CPD-105 | A | A | D | B |
| CPD-106 | A | A | C | B |
| CPD-107 | D | D | D | D |
| CPD-108 | A | A | C | C |
| CPD-109 | A | A | B | B |
| CPD-110 | D | A | D | B |
| CPD-111 | D | A | D | B |
| CPD-112 | D | D | D | D |
| CPD-113 | A | A | C | B |
| CPD-114 | D | D | D | D |
| CPD-115 | A | A | B | B |
| CPD-116 | B | A | C | C |
| CPD-117 | A | A | B | B |
| CPD-118 | A | A | B | B |
| CPD-119 | D | A | D | C |
| CPD-120 | D | A | D | C |
| CPD-121 | D | A | D | C |
| CPD-122 | B | A | C | B |
| CPD-123 | D | A | D | B |
| CPD-124 | A | A | C | C |
| CPD-125 | A | A | C | C |
| CPD-126 | D | A | D | B |
| CPD-127 | D | D | D | D |
| CPD-128 | A | A | B | B |
| CPD-129 | A | A | B | B |
| CPD-130 | A | A | B | B |
| CPD-131 | D | A | D | B |
| CPD-132 | D | B | D | B |
| CPD-133 | B | A | C | B |
| CPD-134 | C | A | D | C |
| CPD-135 | A | A | C | B |
| CPD-136 | A | A | B | B |
| CPD-137 | B | A | C | C |
| CPD-138 | C | A | D | C |
| CPD-139 | A | A | B | B |
| CPD-140 | D | A | D | B |
| CPD-141 | C | C | D | D |
| CPD-142 | C | — | D | — |
| CPD-143 | D | A | D | B |
| CPD-144 | A | A | B | A |
| CPD-145 | D | A | D | B |
| CPD-146 | A | A | B | A |
| CPD-147 | C | A | C | B |
| CPD-148 | A | A | B | A |
| CPD-149 | A | A | C | B |
| CPD-150 | A | A | B | A |
| CPD-151 | A | A | B | A |
| CPD-152 | D | A | C | B |
| CPD-153 | D | D | D | D |
| CPD-154 | D | D | D | D |
| CPD-155 | A | A | B | A |
| CPD-156 | B | A | B | B |
| CPD-157 | A | A | B | A |
| CPD-158 | A | A | B | A |
| CPD-159 | D | A | D | B |
| CPD-160 | A | A | B | A |
| CPD-161 | A | A | B | A |
| CPD-162 | A | A | B | B |
| CPD-163 | A | A | B | B |
| CPD-164 | A | A | B | A |
| CPD-165 | A | A | B | A |
| CPD-166 | A | A | B | B |
| CPD-167 | A | A | A | A |
| CPD-168 | A | A | B | A |
| CPD-169 | D | B | D | C |
| CPD-170 | A | A | B | A |
| CPD-171 | A | A | A | A |
| CPD-172 | A | A | B | B |
| CPD-173 | A | A | A | A |
| CPD-174 | A | A | B | A |
| CPD-175 | D | A | D | B |
| CPD-176 | D | B | D | B |
| CPD-177 | D | B | D | B |
| CPD-178 | A | A | B | B |
| CPD-179 | A | A | B | B |
| CPD-180 | A | A | D | C |
| CPD-181 | A | A | C | B |
| CPD-182 | D | A | C | C |
| CPD-183 | D | B | B | B |
| CPD-184 | D | B | B | B |
| CPD-185 | C | A | D | C |
| CPD-186 | A | A | B | B |
| CPD-187 | D | A | D | B |
| CPD-188 | A | A | B | B |
| CPD-189 | A | A | B | B |
| CPD-190 | A | A | B | B |
| CPD-191 | D | A | D | C |
| CPD-192 | D | D | D | D |
| CPD-193 | A | A | B | B |
| CPD-194 | D | D | D | D |
| CPD-195 | C | A | D | B |
| CPD-196 | D | A | D | C |
| CPD-197 | D | A | D | C |
| CPD-198 | D | A | D | C |
| CPD-199 | A | A | B | B |
| CPD-200 | A | A | B | B |
| CPD-201 | C | A | C | B |
| CPD-202 | A | A | B | B |
| CPD-203 | A | A | B | B |
| CPD-204 | D | A | D | B |
| CPD-205 | A | A | B | B |
| CPD-206 | A | A | B | B |
| CPD-207 | B | A | C | B |
| CPD-208 | A | A | B | A |
| CPD-209 | A | A | C | B |
| CPD-210 | A | A | C | B |
| CPD-211 | A | A | C | C |
| CPD-212 | A | A | C | B |
| CPD-213 | B | A | C | B |
| CPD-214 | D | A | D | C |
| CPD-215 | D | D | D | D |
| CPD-216 | A | A | C | B |
| CPD-217 | B | A | B | B |
| CPD-218 | A | A | B | B |
| CPD-219 | A | A | B | B |
| CPD-220 | D | B | C | B |
| CPD-221 | A | A | B | B |
| CPD-222 | A | A | B | B |
| CPD-223 | A | A | B | B |
| CPD-224 | B | B | C | C |
| CPD-225 | A | A | C | B |
| CPD-226 | D | A | D | C |
| CPD-227 | D | D | D | D |
| CPD-228 | D | A | D | C |
| CPD-229 | C | A | C | B |
| CPD-230 | D | A | D | C |
| CPD-231 | B | A | C | B |
| CPD-232 | A | A | C | B |
| CPD-233 | D | A | D | C |
| CPD-234 | D | D | D | D |
| CPD-235 | A | A | D | B |
| CPD-236 | A | A | C | B |
| CPD-237 | A | A | B | B |
| CPD-238 | C | D | D | D |
| CPD-239 | A | A | C | B |
| CPD-240 | B | A | C | C |
| CPD-241 | A | A | B | B |
| CPD-242 | A | A | B | B |
| CPD-243 | A | A | B | B |
| CPD-244 | A | A | C | B |
| CPD-245 | A | A | C | B |
| CPD-246 | A | A | B | B |
| CPD-247 | B | A | B | B |
| CPD-248 | D | B | D | D |
| CPD-249 | A | A | B | B |
| CPD-250 | A | A | C | B |
| CPD-253 | B | A | D | D |
| CPD-255 | D | A | D | B |
| CPD-256 | D | D | D | D |
| CPD-257 | B | A | D | C |
| CPD-258 | B | A | C | B |
| CPD-259 | D | C | D | C |
| CPD-282 | B | A | D | D |
| CPD-300 | C | B | D | D |
| CPD-301 | A | A | D | D |
| CPD-302 | A | A | D | D |
| CPD-305 | A | A | C | C |
| CPD-306 | A | B | C | C |
| CPD-316 | B | B | C | C |
| CPD-322 | B | A | D | D |
| CPD-323 | A | A | D | D |
| CPD-324 | B | A | D | D |
| CPD-326 | B | B | D | D |
| CPD-328 | B | B | D | D |
| CPD-333 | B | A | D | D |
| CPD-338 | B | B | C | C |
| CPD-341 | B | A | D | D |
| CPD-342 | B | B | D | D |
| CPD-358 | B | B | D | D |
| CPD-359 | B | B | D | D |
| CPD-360 | B | B | D | D |
| CPD-367 | B | B | D | D |
| CPD-384 | B | A | D | D |
| CPD-399 | B | B | D | D |
| CPD-402 | B | B | D | D |
| CPD-411 | B | A | D | D |

A, degradation activity >80%; B, 50%< degradation activity <80%; C, 30%< degradation activity <50%; D, degradation activity <30%; —, not available

### Example 737. Heterobifunctional Compounds Suppressed the Viability of Calu-1, T47D or MCF7 cells (Table 7)

NSCLC Calu-1 or ER+ breast cancer T47D cells were treated with selected heterobifunctional compounds following a 10-point serial dilution for 3 or 6 days, respectively. Heterobifunctional compounds potently suppressed the viability of Calu-1, T47D), or MCF7 cells. The cell viability inhibition results of selected heterobifunctional compounds are set forth in Table 7.

Cells were treated with heterobifunctional compounds following a 10-point serial dilution for 3-6 days. In Calu-1 cells, A: anti-proliferation activity IC₅₀≤10 nM; B: anti-proliferation activity IC₅₀<50 nM, and ≥10 nM; C: anti-proliferation activity IC₅₀≥50 nM. In T47D cells, A: anti-proliferation activity IC₅₀≤50 nM; B: anti-proliferation activity IC₅₀≤100 nM, and ≥50 nM; C: anti-proliferation activity IC₅₀≥100 nM; -, data not available.

TABLE 7
Cellular Anti-proliferation activity (IC₅₀, nM) of selected
heterobifunctional compounds in Calu-1, T47D, or MCF7 cells.

| | GI IC₅₀ | GI IC₅₀ | GI IC₅₀ |
|---|---|---|---|
| | in Calu-1 | in T47D | in MCF-7 |
| Cpd. No. | (nM) | (nM) | (nM) |
| CPD-4 | B | — | B |
| CPD-5 | A | A | A |
| CPD-8 | A | A | A |
| CPD-10 | A | A | B |
| CPD-11 | B | B | A |
| CPD-14 | A | B | A |
| CPD-16 | A | A | A |
| CPD-18 | A | A | A |
| CPD-21 | B | B | C |
| CPD-22 | A | A | B |
| CPD-24 | B | C | C |
| CPD-26 | B | B | C |
| CPD-27 | A | B | B |
| CPD-28 | B | A | — |
| CPD-29 | B | B | — |
| CPD-30 | B | B | C |
| CPD-33 | B | B | C |
| CPD-34 | A | A | A |
| CPD-35 | B | B | — |
| CPD-37 | B | C | — |
| CPD-38 | A | B | C |
| CPD-39 | A | A | C |
| CPD-50 | A | B | A |
| CPD-51 | A | B | B |
| CPD-54 | A | B | A |
| CPD-57 | B | B | — |
| CPD-62 | A | A | A |
| CPD-63 | A | A | A |
| CPD-66 | A | B | C |
| CPD-73 | B | B | B |
| CPD-75 | B | A | — |
| CPD-79 | A | A | A |
| CPD-80 | A | A | — |
| CPD-81 | B | B | — |
| CPD-84 | B | B | C |
| CPD-85 | A | B | — |
| CPD-101 | A | B | A |
| CPD-103 | A | B | C |
| CPD-115 | A | A | C |
| CPD-129 | A | B | C |
| CPD-135 | B | C | A |
| CPD-136 | A | B | C |
| CPD-139 | A | A | — |
| CPD-143 | C | C | C |
| CPD-144 | B | — | — |
| CPD-146 | A | — | A |
| CPD-150 | A | — | B |
| CPD-155 | A | B | C |
| CPD-157 | A | — | B |
| CPD-158 | A | — | A |
| CPD-165 | A | — | C |
| CPD-167 | A | — | A |
| CPD-170 | A | — | B |
| CPD-171 | A | — | A |
| CPD-173 | A | — | A |
| CPD-174 | B | — | C |
| CPD-178 | A | — | B |
| CPD-186 | B | — | C |
| CPD-189 | B | — | C |
| CPD-190 | A | — | B |
| CPD-199 | A | — | B |
| CPD-200 | A | — | B |
| CPD-202 | — | — | A |
| CPD-203 | — | — | A |
| CPD-205 | — | — | A |
| CPD-206 | A | — | C |
| CPD-208 | A | — | A |
| CPD-209 | — | C | — |
| CPD-210 | — | | — |
| CPD-211 | — | | — |
| CPD-212 | — | | — |
| CPD-213 | — | | — |
| CPD-214 | — | | — |
| CPD-215 | — | | — |
| CPD-216 | — | | — |
| CPD-217 | — | | C |
| CPD-218 | — | B | B |
| CPD-219 | — | B | B |
| CPD-220 | — | | — |
| CPD-221 | — | | C |
| CPD-222 | — | A | B |
| CPD-223 | — | | B |
| CPD-224 | — | | A |
| CPD-225 | — | | — |
| CPD-226 | — | | — |
| CPD-227 | — | | — |
| CPD-228 | — | | — |
| CPD-229 | — | | — |
| CPD-230 | — | | — |
| CPD-231 | — | | — |
| CPD-232 | — | | B |
| CPD-233 | — | | — |
| CPD-234 | — | | — |
| CPD-235 | — | | — |
| CPD-236 | — | | — |
| CPD-237 | — | | C |
| CPD-238 | — | | — |
| CPD-239 | — | | — |
| CPD-240 | — | | — |
| CPD-241 | — | | B |
| CPD-242 | — | | C |
| CPD-243 | — | | — |
| CPD-244 | — | | B |
| CPD-245 | — | | — |
| CPD-246 | — | | — |
| CPD-247 | — | | — |
| CPD-248 | — | | — |
| CPD-249 | — | | — |
| CPD-250 | — | | B |
| CPD-251 | — | | C |
| CPD-252 | — | C | — |
| CPD-253 | — | C | A |
| CPD-254 | — | C | C |
| CPD-255 | — | | — |
| CPD-256 | — | | — |
| CPD-257 | — | | — |
| CPD-258 | — | | — |
| CPD-259 | — | | — |
| CPD-260 | — | | C |
| CPD-261 | — | | C |
| CPD-262 | — | | C |
| CPD-263 | — | | C |
| CPD-264 | — | | C |
| CPD-265 | — | C | — |
| CPD-266 | — | C | — |
| CPD-267 | — | | A |
| CPD-268 | — | | A |
| CPD-269 | — | | C |
| CPD-270 | C | | C |
| CPD-271 | C | | C |
| CPD-272 | A | | A |
| CPD-273 | C | | C |
| CPD-274 | B | | C |
| CPD-275 | C | | C |
| CPD-276 | C | | C |
| CPD-277 | A | | C |
| CPD-278 | B | | C |
| CPD-279 | B | | C |
| CPD-280 | A | | B |
| CPD-281 | B | | C |
| CPD-282 | A | | A |
| CPD-283 | A | | B |
| CPD-284 | A | | C |
| CPD-285 | B | | C |
| CPD-286 | C | | C |
| CPD-287 | C | | C |
| CPD-288 | C | | C |
| CPD-289 | C | | C |
| CPD-290 | — | | C |
| CPD-291 | — | | C |
| CPD-292 | — | | C |
| CPD-293 | — | | C |
| CPD-294 | — | | C |
| CPD-295 | — | | C |
| CPD-296 | — | | C |
| CPD-297 | — | | C |
| CPD-298 | — | | C |
| CPD-299 | — | | A |
| CPD-300 | — | | B |
| CPD-301 | — | | A |
| CPD-302 | — | | A |
| CPD-303 | — | | A |
| CPD-304 | — | | C |
| CPD-305 | — | | A |
| CPD-306 | — | | A |
| CPD-307 | — | | C |
| CPD-308 | — | | C |
| CPD-309 | — | | C |
| CPD-310 | — | | C |
| CPD-311 | — | | C |
| CPD-312 | — | | C |
| CPD-313 | — | | C |
| CPD-314 | — | | C |
| CPD-315 | — | | C |
| CPD-316 | — | | A |
| CPD-317 | — | | C |
| CPD-318 | — | | C |
| CPD-319 | — | | C |
| CPD-320 | — | | C |
| CPD-321 | — | | C |
| CPD-322 | — | | A |
| CPD-323 | — | | A |
| CPD-324 | — | | A |
| CPD-325 | — | | C |
| CPD-326 | — | | B |
| CPD-327 | — | | C |
| CPD-328 | — | | A |
| CPD-329 | — | | C |
| CPD-330 | — | | C |
| CPD-331 | — | | C |
| CPD-332 | — | | C |
| CPD-333 | — | | A |
| CPD-334 | — | | C |
| CPD-335 | — | | C |
| CPD-336 | — | | C |
| CPD-337 | — | | C |
| CPD-338 | — | | A |
| CPD-339 | — | | B |
| CPD-340 | — | | C |
| CPD-341 | — | | A |
| CPD-342 | — | | A |
| CPD-343 | — | | B |
| CPD-344 | — | | C |
| CPD-345 | — | | C |
| CPD-346 | — | | C |
| CPD-347 | — | | C |
| CPD-348 | — | | C |
| CPD-349 | — | | C |
| CPD-350 | — | | C |
| CPD-351 | — | | C |
| CPD-352 | — | | C |
| CPD-353 | — | | C |
| CPD-354 | — | | C |
| CPD-355 | — | | B |
| CPD-356 | — | | B |
| CPD-357 | — | | A |
| CPD-358 | — | | B |
| CPD-359 | — | | A |
| CPD-360 | — | | A |
| CPD-361 | — | | C |
| CPD-362 | — | | C |
| CPD-363 | — | | C |
| CPD-364 | — | | C |
| CPD-365 | — | | A |
| CPD-366 | — | | C |
| CPD-367 | — | | A |
| CPD-368 | — | | A |
| CPD-369 | — | | B |
| CPD-370 | — | | A |
| CPD-371 | — | | C |
| CPD-372 | — | | C |
| CPD-373 | — | | C |
| CPD-374 | — | | C |
| CPD-375 | — | | C |
| CPD-376 | — | | C |
| CPD-377 | — | | B |
| CPD-378 | — | | C |
| CPD-379 | — | | C |
| CPD-380 | — | | C |
| CPD-381 | — | | C |
| CPD-382 | — | | B |
| CPD-383 | — | | C |
| CPD-384 | — | | A |
| CPD-385 | — | | B |
| CPD-386 | — | | C |
| CPD-387 | — | | B |
| CPD-388 | — | | C |
| CPD-389 | — | | C |
| CPD-390 | — | | C |
| CPD-391 | — | | C |
| CPD-392 | — | | C |
| CPD-393 | — | | B |
| CPD-394 | — | | C |
| CPD-395 | — | | C |
| CPD-396 | — | | C |
| CPD-397 | — | | A |
| CPD-398 | — | | B |
| CPD-399 | — | | A |
| CPD-400 | — | | B |
| CPD-401 | — | | C |
| CPD-402 | — | | B |
| CPD-403 | — | | B |
| CPD-404 | — | | C |
| CPD-405 | — | | C |
| CPD-406 | — | | A |
| CPD-407 | — | | A |
| CPD-408 | — | | A |
| CPD-409 | — | | A |
| CPD-410 | — | | C |
| CPD-411 | — | | A |
| CPD-412 | — | | C |
| CPD-413 | — | | C |
| CPD-414 | — | | A |
| CPD-415 | — | | A |
| CPD-416 | — | | C |
| CPD-417 | — | | C |
| CPD-418 | — | | A |
| CPD-419 | — | | A |
| CPD-420 | — | | C |
| CPD-421 | — | | C |
| CPD-422 | — | | A |
| CPD-423 | — | | B |
| CPD-424 | — | | C |
| CPD-425 | — | | A |
| CPD-426 | — | | A |
| CPD-427 | — | | A |
| CPD-428 | — | | C |
| CPD-429 | — | | A |
| CPD-430 | — | | C |
| CPD-431 | — | | B |
| CPD-432 | — | | A |
| CPD-433 | — | | A |
| CPD-434 | — | | A |
| CPD-435 | — | | A |
| CPD-436 | — | | A |
| CPD-437 | — | | C |
| CPD-438 | — | | B |
| CPD-439 | — | | A |
| CPD-440 | — | | A |
| CPD-441 | — | | A |
| CPD-442 | — | | C |
| CPD-443 | — | | A |
| CPD-444 | — | | A |
| CPD-445 | — | | A |
| CPD-446 | — | | A |
| CPD-447 | — | | A |
| CPD-448 | — | | A |
| CPD-449 | — | | A |
| CPD-450 | — | | A |
| CPD-451 | — | | A |
| CPD-452 | — | | A |
| CPD-453 | — | | B |
| CPD-454 | B | | C |
| CPD-455 | A | | A |
| CPD-456 | A | | A |
| CPD-457 | A | | A |
| CPD-458 | A | | C |
| CPD-459 | A | | C |
| CPD-460 | A | | C |
| CPD-461 | A | | B |
| CPD-462 | B | | C |
| CPD-463 | A | | A |
| CPD-464 | A | | A |
| CPD-465 | A | | A |
| CPD-466 | A | | B |
| CPD-467 | A | | C |
| CPD-468 | B | | C |
| CPD-469 | A | | A |
| CPD-470 | A | | C |
| CPD-471 | A | | A |
| CPD-472 | A | | A |
| CPD-473 | A | | A |
| CPD-474 | A | | B |
| CPD-475 | A | | C |
| CPD-476 | A | | A |
| CPD-477 | B | | C |
| CPD-478 | A | | A |
| CPD-479 | A | | — |
| CPD-480 | B | | — |
| CPD-481 | A | | A |
| CPD-482 | A | | A |
| CPD-483 | C | | — |
| CPD-484 | B | | — |
| CPD-485 | A | | — |
| CPD-486 | A | | A |
| CPD-487 | A | | A |
| CPD-488 | A | | A |
| CPD-489 | C | | — |
| CPD-490 | C | | — |
| CPD-491 | A | | A |
| CPD-492 | A | | A |
| CPD-493 | A | | — |
| CPD-494 | C | | — |
| CPD-495 | C | | — |
| CPD-496 | A | | — |
| CPD-497 | C | | — |
| CPD-498 | C | | — |
| CPD-499 | A | | A |
| CPD-500 | A | | — |
| CPD-501 | A | | A |
| CPD-502 | B | | — |
| CPD-503 | A | | A |
| CPD-504 | A | | A |
| CPD-505 | A | | A |
| CPD-506 | A | | A |
| CPD-507 | C | | — |
| CPD-508 | C | | — |
| CPD-509 | C | | — |
| CPD-510 | C | | — |
| CPD-511 | C | | — |
| CPD-512 | B | | — |
| CPD-513 | C | | — |
| CPD-514 | C | | — |
| CPD-515 | A | | A |
| CPD-516 | B | | — |
| CPD-517 | C | | — |
| CPD-518 | A | | — |
| CPD-519 | A | | — |
| CPD-520 | A | | A |
| CPD-521 | B | | — |
| CPD-522 | A | | — |
| CPD-523 | C | | — |
| CPD-524 | A | | — |
| CPD-525 | A | | A |
| CPD-526 | A | | A |
| CPD-527 | A | | — |
| CPD-528 | A | | A |
| CPD-529 | A | | A |
| CPD-530 | B | | C |
| CPD-531 | A | | A |
| CPD-532 | A | | A |
| CPD-533 | A | | B |
| CPD-534 | A | | A |
| CPD-535 | B | | B |
| CPD-536 | A | | A |
| CPD-537 | A | | A |
| CPD-538 | A | | B |
| CPD-539 | B | | A |
| CPD-540 | C | | C |
| CPD-541 | A | | A |
| CPD-542 | A | | C |
| CPD-543 | A | | A |
| CPD-544 | A | | A |
| CPD-545 | B | | C |
| CPD-546 | A | | A |
| CPD-547 | A | | A |
| CPD-548 | A | | A |
| CPD-549 | A | | A |
| CPD-550 | A | | A |
| CPD-551 | A | | A |
| CPD-552 | C | | C |
| CPD-553 | C | | C |
| CPD-554 | C | | C |
| CPD-555 | A | | B |
| CPD-556 | A | | A |
| CPD-557 | — | | C |
| CPD-558 | — | | C |
| CPD-559 | B | | C |
| CPD-560 | A | | C |
| CPD-561 | A | | C |
| CPD-564 | B | | C |
| CPD-565 | A | | A |
| CPD-566 | A | | A |
| CPD-567 | A | | B |
| CPD-568 | A | | C |
| CPD-569 | C | | C |
| CPD-570 | A | | A |
| CPD-571 | A | | A |
| CPD-572 | A | | B |
| CPD-573 | — | | A |
| CPD-574 | — | | A |
| CPD-575 | — | | C |
| CPD-576 | — | | C |
| CPD-577 | — | | A |
| CPD-578 | — | | A |
| CPD-579 | — | | A |
| CPD-580 | — | | A |
| CPD-581 | — | | A |
| CPD-582 | — | | A |
| CPD-583 | — | | A |
| CPD-584 | — | | A |
| CPD-585 | — | | A |
| CPD-586 | — | | A |
| CPD-587 | — | | A |
| CPD-588 | — | | A |
| CPD-589 | A | | A |
| CPD-590 | — | | A |
| CPD-591 | — | | A |
| CPD-592 | A | | A |
| CPD-594 | — | | A |
| CPD-595 | — | | A |
| CPD-596 | — | | C |
| CPD-597 | — | | A |
| CPD-599 | — | | A |
| CPD-600 | — | | C |
| CPD-601 | — | | C |
| CPD-602 | — | | C |
| CPD-603 | — | | C |
| CPD-607 | — | | C |
| CPD-608 | — | | C |
| CPD-616 | C | | C |
| CPD-617 | — | | C |
| CPD-618 | — | | C |
| CPD-619 | — | | C |
| CPD-620 | — | | C |
| CPD-621 | — | | C |
| CPD-622 | — | | C |
| CPD-625 | — | | C |
| CPD-626 | — | | C |
| CPD-627 | — | | C |
| CPD-628 | — | | C |
| CPD-629 | — | | C |
| CPD-630 | — | | C |
| CPD-633 | — | | C |
| CPD-634 | — | | C |
| CPD-635 | — | | C |
| CPD-636 | — | | C |
| CPD-639 | — | | C |
| CPD-642 | — | | C |
| CPD-643 | — | | C |
| CPD-644 | — | | C |
| CPD-645 | — | | C |
| CPD-646 | — | | C |
| CPD-657 | — | | C |

Calu-1: A, IC₅₀ < 10 nM; B, 10 nm < IC₅₀ < 50 nM; C, 50 nM < IC₅₀
T47D: A, IC₅₀ < 50 nM; B, 50 nm < IC₅₀ < 100 nM; C, 100 nM < IC₅₀
MCF7: A, IC₅₀ < 50 nM; B, 50 nm < IC₅₀ < 100 nM; C, 100 nM < IC₅₀

### Example 738. Heterobifunctional Compounds Concentration-Dependently Reduced Cyclin D1 Protein Levels (Table 8)

MCF7 cells stably expressing HiBiT tagged target proteins were treated with heterobifunctional compounds at indicated concentrations for 16 hours. Specific exemplary compounds reduced cyclin D1 and CDK4 protein levels at 10 nM and 100 nM.

The cellular protein degradation results of selected heterobifunctional compounds measured by HiBiT are set forth in Table 8.

TABLE 8

| | % Cyclin D1 | % Cyclin D1 | % CDK4 | % CDK4 |
|---|---|---|---|---|
| | Relative | Relative | Relative | Relative |
| | Level | Level | Level | Level |
| Cpd. No. | (10 nM) | (100 nM) | (10 nM) | (100 nM) |
| CPD-253 | A | A | C | B |
| CPD-282 | A | A | B | B |
| CPD-300 | B | B | — | — |
| CPD-301 | A | A | B | B |
| CPD-302 | B | B | B | B |
| CPD-305 | A | A | B | B |
| CPD-306 | A | B | B | B |
| CPD-338 | A | A | B | B |
| CPD-358 | A | A | — | — |
| CPD-360 | A | A | B | B |
| CPD-367 | A | A | | - |
| CPD-368 | A | A | B | B |
| CPD-384 | B | B | B | B |
| CPD-399 | A | A | — | — |
| CPD-402 | A | A | — | — |
| CPD-403 | B | A | — | — |
| CPD-407 | A | A | B | B |
| CPD-414 | A | A | B | B |
| CPD-422 | A | B | C | B |
| CPD-425 | A | A | B | B |
| CPD-429 | B | B | B | B |
| CPD-446 | A | B | B | B |
| CPD-447 | B | A | B | B |
| CPD-448 | B | B | B | B |
| CPD-456 | B | B | B | B |
| CPD-469 | B | B | A | A |
| CPD-472 | B | B | B | A |
| CPD-473 | B | A | C | C |
| CPD-476 | B | — | — | — |
| CPD-478 | B | A | C | B |
| CPD-479 | B | — | — | — |
| CPD-480 | C | — | — | — |
| CPD-481 | B | — | C | C |
| CPD-482 | B | — | — | — |
| CPD-483 | C | — | — | — |
| CPD-484 | C | — | — | — |
| CPD-485 | B | — | — | — |
| CPD-486 | B | — | — | — |
| CPD-487 | B | — | B | B |
| CPD-488 | B | — | — | — |
| CPD-489 | C | — | — | — |
| CPD-490 | C | — | — | — |
| CPD-491 | B | — | C | C |
| CPD-492 | A | A | C | C |
| CPD-493 | B | — | — | — |
| CPD-494 | C | — | — | — |
| CPD-495 | C | — | — | — |
| CPD-496 | B | — | — | — |
| CPD-497 | C | — | — | — |
| CPD-498 | C | — | — | — |
| CPD-499 | A | A | C | B |
| CPD-500 | B | — | — | — |
| CPD-501 | B | — | C | C |
| CPD-502 | C | — | — | — |
| CPD-503 | B | — | B | B |
| CPD-504 | B | — | — | — |
| CPD-505 | A | A | C | C |
| CPD-506 | A | A | C | C |
| CPD-507 | C | — | — | — |
| CPD-508 | C | — | — | — |
| CPD-509 | C | — | — | — |
| CPD-510 | C | — | — | — |
| CPD-511 | C | — | — | — |
| CPD-512 | B | — | — | — |
| CPD-513 | C | — | — | — |
| CPD-514 | B | — | — | — |
| CPD-515 | B | — | C | C |
| CPD-516 | C | — | — | — |
| CPD-517 | C | — | — | — |
| CPD-518 | B | — | — | — |
| CPD-519 | B | — | — | — |
| CPD-520 | B | — | C | C |
| CPD-521 | B | — | — | — |
| CPD-522 | B | — | — | — |
| CPD-523 | C | — | — | — |
| CPD-524 | B | — | — | — |
| CPD-525 | B | — | C | C |
| CPD-526 | B | — | — | — |
| CPD-527 | B | — | — | — |
| CPD-528 | B | — | — | — |
| CPD-529 | A | A | C | C |
| CPD-530 | B | — | — | — |
| CPD-531 | A | A | B | B |
| CPD-532 | A | A | B | B |
| CPD-533 | A | A | — | — |
| CPD-534 | A | A | — | — |
| CPD-535 | B | — | — | — |
| CPD-536 | A | A | C | B |
| CPD-537 | A | A | C | C |
| CPD-538 | B | — | — | — |
| CPD-539 | B | — | — | — |
| CPD-540 | C | — | — | — |
| CPD-541 | B | — | — | — |
| CPD-542 | C | — | — | — |
| CPD-543 | B | — | — | — |
| CPD-544 | B | — | — | — |
| CPD-545 | C | — | — | — |
| CPD-546 | B | A | — | — |
| CPD-547 | B | — | — | — |
| CPD-548 | B | — | — | — |
| CPD-549 | B | — | — | — |
| CPD-550 | B | — | C | C |
| CPD-551 | — | — | C | B |
| CPD-552 | C | — | — | — |
| CPD-553 | C | — | — | — |
| CPD-554 | C | — | — | — |
| CPD-555 | C | — | — | — |
| CPD-556 | B | — | — | — |
| CPD-557 | C | A | C | C |
| CPD-558 | C | A | C | C |
| CPD-559 | B | A | — | — |
| CPD-560 | C | B | — | — |
| CPD-561 | C | B | — | — |
| CPD-562 | C | C | C | C |
| CPD-563 | C | C | C | C |
| CPD-564 | C | B | — | — |
| CPD-565 | B | B | C | B |
| CPD-566 | B | B | — | — |
| CPD-567 | B | B | — | — |
| CPD-568 | B | B | — | — |
| CPD-569 | C | B | — | — |
| CPD-570 | B | B | C | B |
| CPD-571 | A | A | — | — |
| CPD-572 | B | A | — | — |
| CPD-573 | B | B | B | B |
| CPD-574 | C | C | C | B |
| CPD-575 | C | C | C | B |
| CPD-576 | B | B | B | B |
| CPD-577 | C | B | C | B |
| CPD-578 | B | B | B | B |
| CPD-579 | B | B | B | B |
| CPD-580 | C | B | B | B |
| CPD-581 | B | B | B | B |
| CPD-582 | B | B | B | B |
| CPD-583 | B | B | B | B |
| CPD-584 | B | B | B | B |
| CPD-585 | B | B | B | B |
| CPD-586 | B | A | B | B |
| CPD-587 | B | B | B | B |
| CPD-588 | B | B | B | B |
| CPD-589 | C | B | B | B |
| CPD-590 | C | C | B | B |
| CPD-591 | B | B | B | B |
| CPD-592 | B | B | B | B |
| CPD-593 | C | C | C | C |
| CPD-594 | B | B | B | B |
| CPD-595 | C | C | C | B |
| CPD-596 | C | C | B | C |
| CPD-597 | C | B | C | B |
| CPD-598 | C | C | B | C |
| CPD-599 | C | B | B | B |
| CPD-600 | C | C | B | B |
| CPD-601 | C | C | B | B |
| CPD-602 | C | C | B | B |
| CPD-603 | C | C | B | B |
| CPD-604 | C | C | C | C |
| CPD-605 | C | C | C | C |
| CPD-606 | C | C | C | C |
| CPD-607 | C | C | C | C |
| CPD-608 | C | C | C | B |
| CPD-609 | C | C | C | C |
| CPD-610 | C | C | C | C |
| CPD-611 | C | C | C | C |
| CPD-612 | C | C | C | C |
| CPD-613 | C | C | C | C |
| CPD-614 | C | C | C | C |
| CPD-615 | C | C | C | C |
| CPD-616 | C | C | C | C |
| CPD-617 | C | C | C | C |
| CPD-618 | C | C | C | B |
| CPD-619 | C | C | B | B |
| CPD-620 | C | C | B | B |
| CPD-621 | B | C | B | B |
| CPD-622 | C | C | B | B |
| CPD-623 | C | C | C | C |
| CPD-624 | C | C | B | B |
| CPD-625 | C | C | B | B |
| CPD-626 | C | C | B | B |
| CPD-627 | C | C | B | B |
| CPD-628 | B | C | B | B |
| CPD-629 | B | C | B | B |
| CPD-630 | B | C | B | B |
| CPD-631 | C | C | C | C |
| CPD-632 | C | C | C | C |
| CPD-633 | C | C | B | C |
| CPD-634 | C | C | B | B |
| CPD-635 | C | C | B | B |
| CPD-636 | C | C | B | B |
| CPD-637 | C | C | C | C |
| CPD-638 | C | C | C | C |
| CPD-639 | C | C | C | C |
| CPD-640 | C | C | C | B |
| CPD-641 | C | C | C | C |
| CPD-642 | C | C | B | B |
| CPD-643 | C | C | C | B |
| CPD-644 | C | C | C | C |
| CPD-645 | C | C | C | C |
| CPD-646 | C | C | C | C |
| CPD-647 | C | C | C | C |
| CPD-648 | C | C | C | C |
| CPD-649 | C | C | C | C |
| CPD-650 | C | C | C | C |
| CPD-651 | C | C | C | C |
| CPD-652 | C | C | C | C |
| CPD-653 | C | C | C | C |
| CPD-654 | C | C | C | C |
| CPD-655 | C | C | C | C |
| CPD-656 | C | C | C | C |
| CPD-657 | C | C | C | C |
| CPD-658 | C | C | C | C |
| CPD-659 | C | C | C | C |
| CPD-660 | C | C | C | C |
| CPD-661 | C | C | C | C |
| CPD-662 | C | C | C | C |
| CPD-663 | C | C | C | B |
| CPD-664 | C | C | C | C |
| CPD-665 | C | C | B | C |
| CPD-666 | C | C | C | C |
| CPD-667 | C | C | C | C |
| CPD-668 | C | C | C | C |
| CPD-669 | C | C | C | C |
| CPD-670 | C | C | C | C |
| CPD-671 | C | C | C | C |
| CPD-672 | C | C | C | C |
| CPD-673 | C | C | C | C |
| CPD-674 | C | C | C | C |
| CPD-675 | C | C | C | C |
| CPD-676 | C | C | C | C |
| CPD-677 | C | C | C | C |
| CPD-678 | C | C | C | C |
| CPD-679 | C | C | C | C |

A, Relative protein level ≤55%; B, 55%< Relative protein level ≤85%; C, Relative protein level >85%

### Example 739. Heterobifunctional Compound-Mediated CCND Degradation Depended on Binding to CDK4 (FIG. 2A-2B)

T47D cells were treated with a dose range of heterobifunctional compound CPD-10, and its negative control compound CPD-143 for 48 hours (FIG. 2A). Negative control compound CPD-143 showed much weaker degradation potencies and downstream inhibition potencies compared with its corresponding active heterobifunctional compound CPD-10.

Calu-1, MCF7 or T47D cells were seeded in 96-well plates and treated with CPD-10, or CPD-143 following a 10-point serial dilution for 3-6 days (FIG. 2B). Negative control compound CPD-143 showed much weaker cellular anti-proliferation activities compared to its corresponding active heterobifunctional compound CPD-10.

### Example 740. CPD-10 Caused Cell Apoptosis in ER⁺ Breast Cancer Lines (FIG. 3A-3B)

T47D cells were treated with DMSO, palbociclib, heterobifunctional compound CPD-10 for 6 days at doses approximating IC₅₀ and IC₉₀ concentrations as indicated (FIG. 3A). HCC1428 cells were treated with DMSO, palbociclib, heterobifunctional compound CPD-10, or its negative control compound CPD-143 for 6 days at doses approximating IC₉₀ concentrations as indicated (FIG. 3B). Cells were harvested by trypsinization, staining was carried out using the Annexin V Apoptosis Detection Kit. Flow cytometric analysis showed that CPD-10 cause significant cell apoptosis (Annexin V⁺ population: 20.9% at 15 nM, 62.2% at 200 nM in T47D; 55.8% in HCC1428), while palbociclib or CPD-143 showed much less effect on cell apoptosis.

### Example 741. CPD-10 and CPD-39 Suppressed Cell Viability in T47D Palbociclib-Resistant Model (FIG. 4)

T47D cells were cultured long-term to resistance in the presence of 1 μM Palbociclib. Palbo resistance was determined by CellTiter-Lumi cell viability assay. T47D parental or palbo-resistant cells were treated with palbociclib, or heterobifunctional compounds CPD-10 or CPD-39 following a 10-point serial dilution for 6 days. Cell viability data showed that CPD-10 and CPD-39 remained effective in T47D palbo-resistant model.

### Example 742. The Pharmacokinetics of CPD-39 (FIG. 5)

A single dose of CPD-39 via 1 mg/kg intravenous (i.v.) injection or 10 mg/kg oral (p.o.) gavage was evaluated in mice. Plasma concentration of CPD-39 reported at each time points post dosing (5 min, 15 min, 30 min, 1 h, 2 h, 4 h, and 8 h via i.v.; 15 min, 30 min, 1 h, 2 h, 4 h, 6 h, 8 h, and 12 h via p.o.) represents the mean value derived from 3 experimental animals. Data showed around 15% oral bioavailability of CPD-39 over 12 hours.

### Example 743. Materials and Methods of Experiments Described Herein

Antibody and Reagent Anti-cyclin D1 (29785), anti-cyclin D2 (3741S), anti-cyclin D3 (2936S), anti-CDK4 (127905), anti-CDK6 (3136S), anti-phospho-Rb (8516S), anti-FoxM1 (54365), anti-cyclinA2(4656S), anti-vinculin antibody (187995) antibodies were purchased from Cell Signaling Technology. HRP-conjugated anti-α-tubulin antibody and anti-α-GAPDH antibody were purchased from GNI. Media, and other cell culture reagents were purchased from Thermo Fisher Scientific. CellTiter-Glo Luminescent Assay kit was purchased from Promega.

### Cell Culture and Transfection

Calu-1, T47D, MCF7, HCC1428 and other cells were cultured at 37° C. with 5% CO2 in RPMI 1640 or DMEM medium supplemented with 10% fetal bovine serum. Cells were authenticated using the short tandem repeat (STR) assays. Mycoplasma test results were negative. For palbo-resistant cell model, T47D cells were cultured long-term to resistance in the presence of 1 μM Palbociclib. Cells were deemed resistant when growing in the presence of Palbo at the same rate as parental cells.

### Immunoblotting

Cultured cells were washed with cold PBS once and lysed in cold RIPA buffer supplemented with protease inhibitors and phosphatase inhibitors (Beyotime Biotechnology). The solutions were then incubated at 4 [symbol] for 30 min with gentle agitation to fully lysed cells. Cell lysates were centrifuged at 13,000 rpm for 10 min at 4 [symbol] and pellets were discarded. Total protein concentrations in the lysates were determined following BCA assays (Beyotime Biotechnology). Cell lysates were mixed with Laemmli loading buffer and heated at 99° C. for 5 min. Proteins were resolved on SDS-PAGE and visualized using Western ECL Substrate kits on a ChemiDoc MP Imaging system (Bio-Rad). Protein bands were quantitated using the Image Lab software provided by Bio-Rad.

### Cell Viability Assay

Cells were seeded at a density of 2,000-5,000 cells per well in 96-well assay plates and treated with test compounds following a 10-point serial dilution for 3-6 days. Cell viability was determined using the CellTiter-Lumi assay kit according to the manufacturer's instructions. The dose-response curves were determined and IC₅₀ values were calculated using the GraphPad Prism software following a nonlinear regression (least squares fit) method. Data presented was mean±standard deviation (SD)unless otherwise indicated.

### HiBiT Assay

MCF7 cells stably expressing HiBiT tagged target proteins were seeded at a density of 5,000 cells per well in 384-well assay plates and treated with test compounds at indicated concentrations for 16 hours, followed by lysed with Nano-Glo HiBiT Lytic detection reagents according to the manufacturer's instructions (Promega, N3030). Luminescence was measured on a Tecan Spark microplate reader.

### Flow Cytometry

Assays were performed on the CytoFLEX Cytometer (Beckman Coulter), and data were analyzed using CytExpert software. For cell death and apoptosis analysis, cells were harvested by trypsinization, staining was carried out using the Annexin V Apoptosis Detection Kit (BD Biosciences). Briefly, cells were resuspended in 1× binding buffer and incubated with fluorochrome-conjugated Annexin V and 7-AAD for 15 min in darkness at room temperature.

### Mouse Pharmacokinetic (PK) Studies

Standard PK studies were conducted using male Swiss Albino mice by Sai Life Sciences. A single dose of test compound via intravenous (i.v.) injection or oral (p.o.) gavage was evaluated in fed mice. Plasma concentration of test compound reported at each time points represents the mean value derived from 3 experimental animals. Error bars represent +SD.

1. A heterobifunctional compound of Formula (I): 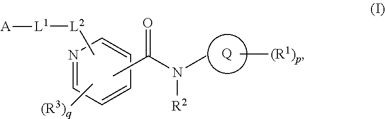
or a pharmaceutically acceptable salt thereof, wherein:
A is a protein binding moiety (PBM) capable of binding to CCND, CDK4 and/or CDK6;
L¹ is a bond or a bivalent linker;
L² is a bond, —NH—, —N(C₁-C₃ alkyl)-, —NHC(O)—, —N(C₁-C₃ alkyl)C(O)—, —C(O)NH—, —C(O)N(C₁-C₃ alkyl)-, —O—, —C₁-C₄-alkylene-, —C₂-C₄-alkenylene- or —C₂-C₄-alkynylene-;
Q is C₆-C₁₀ aryl, 5-10 membered heteroaryl, C₃-C₁₀ cycloalkyl or 4-10 membered heterocyclyl;
each R¹ is independently R^{1A}, R^{1B}, R^{1C}, R^{1D} or R^{1E};
each R^{1A} is independently H, C₁-C₆ alkyl, C₃-C₆ cycloalkyl, or C₁-C₆ heteroalkyl, where each said C₁-C₆ alkyl, C₃-C₆ cycloalkyl, or C₁-C₆ heteroalkyl is optionally substituted by one or more R^{4A};
each R^{1B}, R^{1C}, R^{1D} and R^{1E} is independently H, C₁-C₆ alkyl, C₁-C₆ heteroalkyl, C₁-C₆ alkoxy, OH, D, halo, CN, NO₂, NR^{5A}R^{5B}, C(═O)R^{5A}, C(═O)OR^{5A}, OC(═O)R^{5A}, C(O)NR^{5A}R^{5B}, N(R^{5A})C(O)R^{5B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl, or C₁-C₆ alkoxy is optionally substituted by one or more R^{4B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{4C};
R² is H or C₁-C₃ alkyl;
each R³ is independently R^{3A}, R^{3B} or R^{3C}; or two R³ may be taken together with the atoms to which they are attached to form a C₃-C₁₀ cycloalkyl, 4-10 membered heterocyclyl, C₆-C₁₀ aryl or 5-10 membered heteroaryl, where each said C₃-C₁₀ cycloalkyl, 4-10 membered heterocyclyl, C₆-C₁₀ aryl or 5-10 membered heteroaryl is optionally substituted by one or more R^{6A};
each R^{3A}, R^{3B} and R^{3C} is independently C₁-C₆ alkyl, C₁-C₆ heteroalkyl, C₁-C₆ alkoxy, OH, D, halo, CN, NO₂, NR^{7A}R^{7B}, C(═O)R^{7A}, C(═O)OR^{7A}, OC(═O)R^{7A}, C(O)NR^{7A}R^{7B}, N(R^{7A})C(O)R^{7B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{6B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{6C};
each R^{4A}, R^{4B} and R^{6B} is independently C₁-C₆ alkoxy, oxo, OH, D, halo, CN or NR^{8A}R^{8B};
each R^{4C}, R^{6A} and R^{6C} is independently C₁-C₄ alkyl, C₁-C₆ alkoxy, oxo, OH, D, halo, CN or NR^{9A}R^{9B};
each R^{5A}, R^{5B}, R^{7A}, R^{7B}, R^{8A}, R^{8B}, R^{9A} and R^{9B} is independently H, C₁-C₆ alkyl, C₁-C₆ heteroalkyl, C₃-C₁₀ cycloalkyl, 4-10 membered heterocyclyl, C₆-C₁₀ aryl or 5-10 membered heteroaryl, where each said C₁-C₆ alkyl or C₁-C₆ heteroalkyl is optionally substituted by one or more R^{10A}, and each said C₃-C₁₀ cycloalkyl, 4-10 membered heterocyclyl, C₆-C₁₀ aryl or 5-10 membered heteroaryl is optionally substituted by one or more R^{10B}; or
R^{5A} and R^{5B}, R^{7A} and R^{7B}, R^{8A} and R^{8B}, or R^{9A} and R^{9B} may be taken together with the N-atom to which they are attached to form a 4-10 membered heterocyclyl ring or 5-10 membered heteroaryl ring, where each said 4-10 membered heterocyclyl or 5-10 membered heteroaryl is optionally substituted by one or more R^{10C};
each R^{10A} is independently C₁-C₆ alkoxy, oxo, OH, D, halo, CN or NR^{11A}R^{11B};
each R^{10B} and R^{10C} is independently C₁-C₄ alkyl, C₁-C₆ alkoxy, oxo, OH, D, halo, CN or NR^{12A}R^{12B};
each R^{11A}, R^{11B}, R^{12A} and R^{12B} is independently H, C₁-C₄ alkyl, or C₃-C₆ cycloalkyl;
p is an integer from 0 to 4; and
q is an integer from 0 to 3;
with the proviso the compound of Formula (I) is not:
2-(3-(2-(2-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)ethoxy)ethoxy)propanamido)-N-(4,5-dimethylthiazol-2-yl)-6-methylnicotinamide; or
2-((7-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)acetamido)heptyl)amino)-N-(4,5-dimethylthiazol-2-yl)-6-methylnicotinamide.

2. The heterobifunctional compound of claim 1, having the structure of Formula (II): 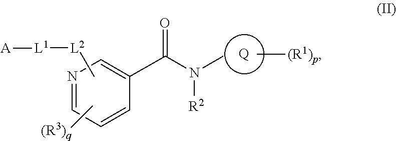
or a pharmaceutically acceptable salt thereof.

3. The heterobifunctional compound of claim 1 or 2, having the structure of Formula (III): 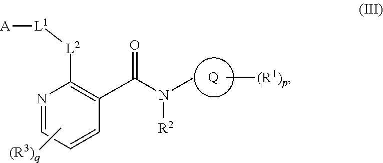
or a pharmaceutically acceptable salt thereof.

4. The heterobifunctional compound of any one of claims 1 to 3, having the structure of Formula (IV): 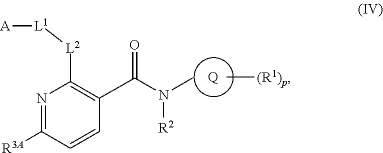
or a pharmaceutically acceptable salt thereof.

5. The heterobifunctional compound of any one of claims 1 to 4, or a pharmaceutically acceptable salt thereof, wherein Q is selected from the group consisting of: 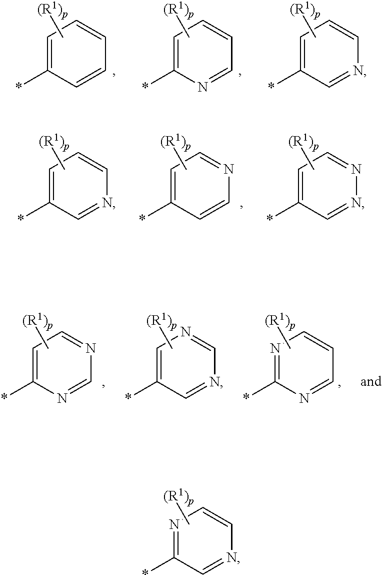
wherein * represents the point of attachment.

6. The heterobifunctional compound of any one of claims 1 to 4, or a pharmaceutically acceptable salt thereof, wherein Q is selected from the group consisting of: 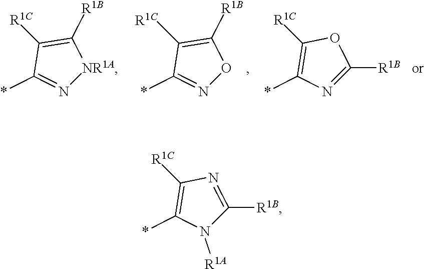
wherein * represents the point of attachment.

7. The heterobifunctional compound of claim 3, having the structure of Formula (V): 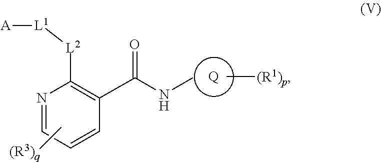
or a pharmaceutically acceptable salt thereof, wherein:
Q is a C₆ aryl or a 6-membered heteroaryl.

8. The heterobifunctional compound of claim 7, having the structure of Formula (V-A), (V-B), (V-C) or (V-D): 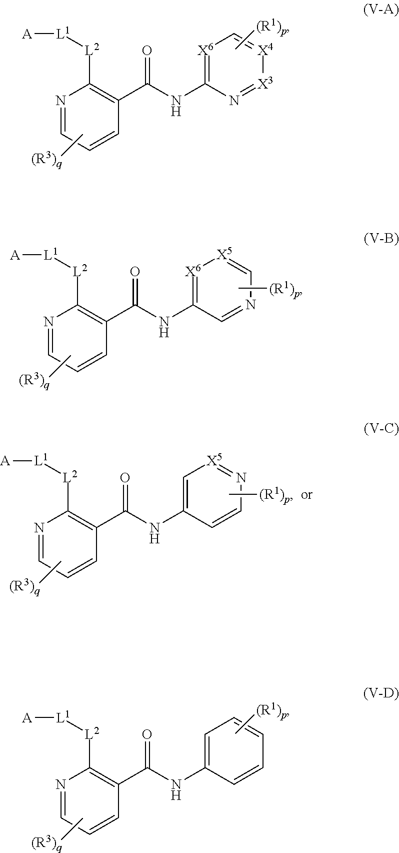
or a pharmaceutically acceptable salt thereof, wherein:
X³ is N or CR^{1B};
X⁴ is N or CR^{1C};
X⁵ is N or CR^{1D}; and
X⁶ is N or CR^{1E}.

9. The heterobifunctional compound of claim 3, having the structure of Formula (VI): 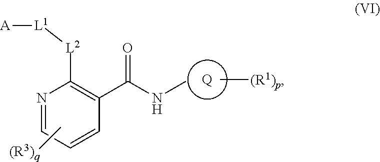
or a pharmaceutically acceptable salt thereof, wherein:
Q is a 5-membered heteroaryl.

10. The heterobifunctional compound of claim 4, having the structure of Formula (VII): 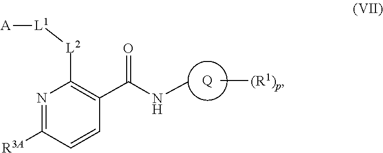
or a pharmaceutically acceptable salt thereof, wherein:
Q is a C₆ aryl or a 6-membered heteroaryl.

11. The heterobifunctional compound of claim 10, having the structure of Formula (VII-A), (VII-B), (VII-C) or (VII-D): 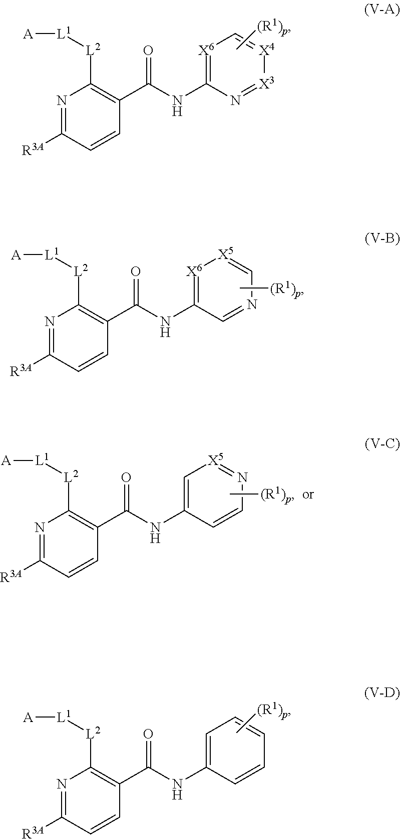
or a pharmaceutically acceptable salt thereof, wherein:
X³ is N or CR^{1B};
X⁴ is N or CR^{1C};
X⁵ is N or CR^{1D}; and
X⁶ is N or CR^{1E}.

12. The heterobifunctional compound of claim 4, having the structure of Formula (VIII): 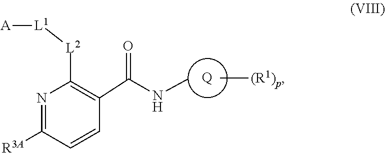
or a pharmaceutically acceptable salt thereof, wherein:
Q is a 5-membered heteroaryl.

13. The heterobifunctional compound of any one of claims 1 to 12, wherein A is a PBM having the structure of Formula (A): 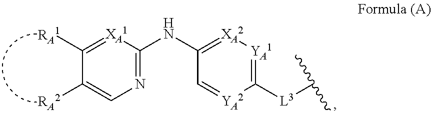
or a pharmaceutically acceptable salt thereof, wherein:
X_{A}¹, X_{A}², Y_{A}¹, and Y_{A}² are each independently CR_{A}⁴ or N;
R_{A}¹ is NR_{A}⁵R_{A}⁶, N(R_{A}⁵)C(═O)R_{A}⁶, optionally substituted C₆-C₁₂ aryl, or optionally substituted 5-13 membered heteroaryl;
R_{A}² is hydrogen, halogen, CN, NO₂, C₁-C₈ alkyl, C₁-C₈ haloalkyl, C₁-C₈ alkoxy, C₁-C₈ heteroalkyl, C₃-C₈ cycloalkyl, or 3-8 membered heterocyclyl; or
R_{A}¹ and R_{A}², together with the atom(s) to which they are attached optionally form an optionally substituted C₃-C₈ cycloalkyl, 3-8 membered heterocyclyl, C₆-C₁₂ aryl or 5-13 membered heteroaryl;
L¹ is a divalent group selected from —R_{A}^{3A}-R_{A}^{3B}-, wherein R_{A}^{3A} and R_{A}^{3B} are each independently a bond, —O—, —S—, —NR_{A}⁷—, —C(═O)—, —C(═O)NR_{A}⁷-, —S(═O)—, —S(═O)NR_{A}⁷—, —S(═O)₂—, —S(═O)₂NR_{A}⁷—, C₁-C₈ alkylene, C₂-C₈ alkenylene, C₂-C₈ alkynylene, C₁-C₈ heteroalkylene, C₂-C₈ heteroalkenylene, C₁-C₈ haloalkylene, C₃-C₁₂ cycloalkylene, 3-12 membered heterocyclene, C₆-C₁₂ arylene, or 5-13 membered heteroarylene, provided two —O— and/or —S— are not contiguous;
each R_{A}⁴ is independently selected from hydrogen, halogen, CN, NO₂, NR_{A}⁸R_{A}⁹, —C(═O)R_{A}¹⁰, —C(═O)OR_{A}¹⁰, —C(═O)NR_{A}⁸R_{A}⁹, —NR_{A}⁸C(═O)R_{A}¹⁰, C₁-C₈ alkyl, C₁-C₈ haloalkyl, C₁-C₈ alkoxy, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl;
R_{A}⁵ and R_{A}⁶ are independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl, or 5-13 membered heteroaryl; or
R_{A}⁵ and R_{A}⁶ together with the atom(s) to which they are connected optionally form a 3-20 membered heterocyclyl ring; and
R_{A}⁷, R_{A}⁸, R_{A}⁹ and R_{A}¹⁰ are each independently selected from hydrogen, C₁-C₈ alkyl, C₁-C₈ haloalkyl, C₁-C₈ alkoxyalkyl, C₁-C₈ heteroalkyl, C₃-C₈ cycloalkyl, 3-8 membered heterocyclyl, C₆-C₁₂ aryl or 5-13 membered heteroaryl; or R_{A}⁸ and R_{A}⁹ together with the atom(s) to which they are connected optionally form a 3-20 membered heterocyclyl ring.

14. The heterobifunctional compound of claim 13, wherein A is a PBM of Formula (A) having the structure of Formula (A1), (A2), or (A3): 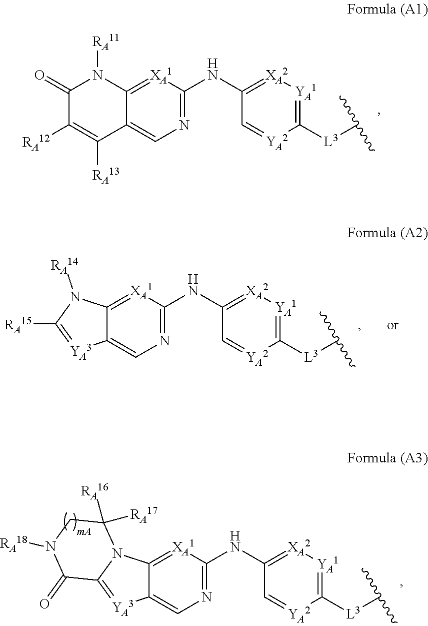
or a pharmaceutically acceptable salt thereof, wherein:
Y_{A}³ is CR_{A}¹⁹ or N;
R_{A}¹¹, R_{A}¹⁴ and R_{A}¹⁸ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, or 3-8 membered heterocyclyl, C₆-C₁₂ aryl, or 5-13 membered heteroaryl;
R_{A}¹² and R_{A}¹⁵ are each independently selected from R_{A}²⁰, COR_{A}²⁰, CO₂R_{A}²⁰, or CONR_{A}²⁰R_{A}²¹, wherein R_{A}²⁰ and R_{A}²¹ are independently selected from hydrogen, halogen, N, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or 3-8 membered heterocyclyl, or R_{A}²⁰ and R_{A}²¹, together with the atom(s) to which they are connected optionally form a 3-20 membered heterocyclyl ring;
R_{A}¹³ is selected from hydrogen, halogen, C₁-C₈ alkyl, C₁-C₈ haloalkyl, C₁-C₈ alkoxy, C₁-C₈ alkylamino, C₁-C₈ heteroalkyl, C₃-C₈ cycloalkyl, or 3-8 membered heterocyclyl;
R_{A}¹⁶ and R_{A}¹⁷ are each independently selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, 3-8 membered heterocyclyl, C₆-C₁₂ aryl, or 5-13 membered heteroaryl; or
R_{A}¹⁶ and R_{A}¹⁷, together with the atom(s) to which they are connected optionally form C₃-C₈ cycloalkyl, or 3-8 membered heterocyclyl; and
R_{A}¹⁹ are independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or 3-8 membered heterocyclyl; and
m_{A} is 0, 1, or 2.

15. The heterobifunctional compound of claim 13, wherein A is a PBM of Formula (A) having the structure of Formula (A4): 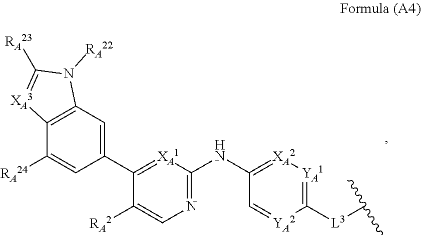
or a pharmaceutically acceptable salt thereof, wherein:
X_{A}³ is CR_{A}²⁵ or N;
R_{A}²² is selected from hydrogen, C₁-C₈ alkyl, C₁-C₈ haloalkyl, C₁-C₈ hydroxyalkyl, C₁-C₈ alkoxyalkyl, C₁-C₈ heteroalkyl, C₂-C₈ alkenyl, C₂-C₈ alkynyl, C₃-C₈ cycloalkyl, C₂-C₈ heterocyclyl, C₆-C₁₂ aryl, or 3-13 membered heteroaryl; and
R_{A}²³, R_{A}²⁴ and R_{A}²⁵ are each independently selected from hydrogen, halogen, CN, NO₂, C₁-C₈ alkyl, C₁-C₈ haloalkyl, C₁-C₈ hydroxyalkyl, C₁-C₈ alkoxyalkyl, C₁-C₈ heteroalkyl, C₁-C₈ alkoxy, C₁-C₈ alkylamino, C₂-C₈ alkenyl, C₂-C₈ alkynyl, C₃-C₈ cycloalkyl, or C₂-C₈ heterocyclyl.

16. The heterobifunctional compound of any one of claims 1 to 12, wherein A is a PBM of Formula (A) having the structure of any of Formulae (A5), (A6), (A7), (A8) or (A9):

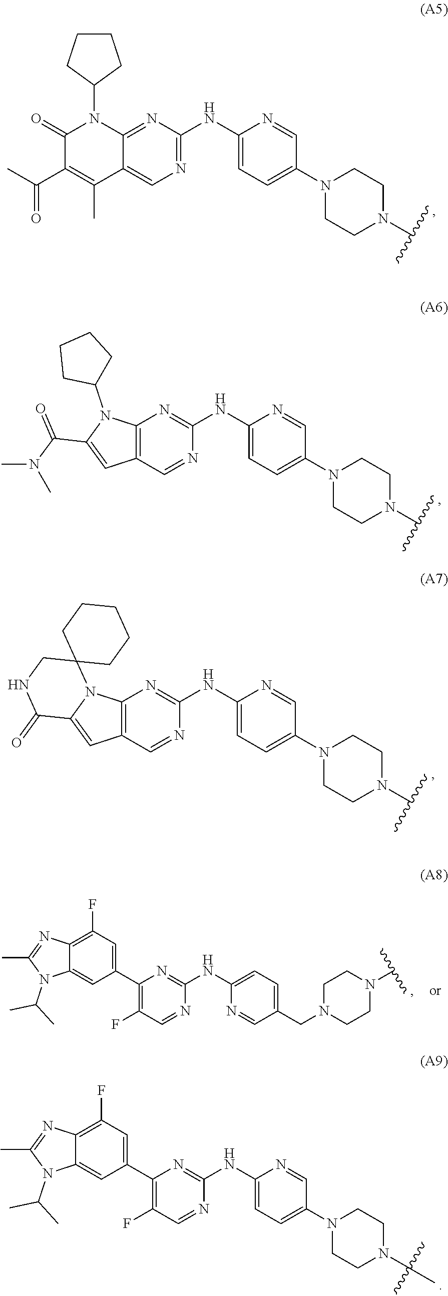 [SMILES: *N1CCN(c2ccc(Nc3ncc4c(C)c(C(C)=O)c(=O)n(C5CCCC5)c4n3)nc2)CC1 ; *N1CCN(c2ccc(Nc3ncc4cc(C(=O)N(C)C)n(C5CCCC5)c4n3)nc2)CC1 ; *N1CCN(c2ccc(Nc3ncc4cc5n(c4n3)C3(CCCCC3)CNC5=O)nc2)CC1 ; *N1CCN(Cc2ccc(Nc3ncc(F)c(-c4cc(F)c5nc(C)n(C(C)C)c5c4)n3)nc2)CC1 ; *N1CCN(c2ccc(Nc3ncc(F)c(-c4cc(F)c5nc(C)n(C(C)C)c5c4)n3)nc2)CC1]

17. The heterobifunctional compound of any one of claims 1 to 16, or a pharmaceutically acceptable salt thereof, wherein L is a bond or a bivalent linker of Formula (L-1): 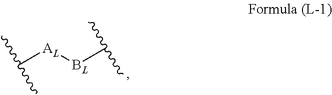
wherein:
each A_{L} and B_{L} is a bivalent moiety independently selected from the group consisting of a bond, R_{L}^{a}, R_{L}^{a}-R_{L}^{b}, R_{L}^{a}C(O)R_{L}^{b}, R_{L}^{a}C(O)OR_{L}^{b}, R_{L}^{a}OC(O)R_{L}^{b}, R_{L}^{a}C(O)N(R_{L}¹)R_{L}^{b}, R_{L}^{a}N(R_{L}¹)C(O)R_{L}^{b}, R_{L}^{a}C(S)N(R_{L}¹)R_{L}^{b}, R_{L}^{a}N(R_{L}¹)C(S)R_{L}^{b}, R_{L}^{a}OR_{L}^{b}, R_{L}^{a}SR_{L}^{b}, R_{L}^{a}SOR_{L}^{b}, R_{L}^{a}SO₂R_{L}^{b}, R_{L}^{a}SO₂N(R_{L}¹)R_{L}^{b}, R_{L}^{a}N(R_{L}¹)SO₂R_{L}^{b}, R_{L}^{a}N(R_{L}¹)R_{L}^{b}, and R_{L}^{a}N(R_{L}¹)C(O)N(R_{L}²)R_{L}^{b};
each R_{L}^{a} and R_{L}^{b} is independently selected from the group consisting of a bond, R_{L}^{r}, (C₁-C₈ alkylene)-R_{L}^{r}, R_{L}^{r}-(C₁-C₈ alkylene), (C₁-C₈ alkylene)-R_{L}^{r}—(C₁-C₈ alkylene), C₁-C₈ alkylene, C₂-C₈ alkenylene, C₂-C₈ alkynylene, C₂-C₈ heteroalkylene, C₃-C₈ heteroalkenylene, and C₃-C₈ heteroalkynylene, where each said C₁-C₈ alkylene, C₂-C₈ alkenylene, C₂-C₈ alkynylene, C₂-C₈ heteroalkylene, C₃-C₈ heteroalkenylene, or C₃-C₈ heteroalkynylene moiety is optionally substituted by one or more R_{L}^{c};
each R_{L}^{r} is independently selected from the group consisting of C₃-C₁₂ cycloalkylene, 3-12 membered heterocyclene, C₆-C₁₂ arylene, and 5-13 membered heteroarylene, where each said C₃-C₁₂ cycloalkylene or 3-12 membered heterocyclene is optionally substituted by one or more R_{L}^{d}, and each said C₆-C₁₂ arylene or 5-13 membered heteroarylene is optionally substituted by one or more R_{L}^{e};
each R_{L}¹ and R_{L}² is independently selected from the group consisting of H, C₁-C₈ alkyl, C₂-C₈ alkenyl, C₂-C₈ alkynyl, C₃-C₁₂ cycloalkyl, 3-12 membered heterocyclyl, C₆-C₁₂ aryl, and 5-13 membered heteroaryl, where each said C₁-C₈ alkyl, C₂-C₈ alkenyl or C₂-C₈ alkynyl is optionally substituted by one or more R_{L}^{f}, each said C₃-C₁₂ cycloalkyl or 3-12 membered heterocyclyl is optionally substituted by one or more R_{L}^{g}, and each said C₆-C₁₂ aryl or 5-13 membered heteroaryl is optionally substituted by one or more R_{L}^{h}; or
R_{L}^{a} and R_{L}^{b}, R_{L}¹ and R_{L}², R_{L}^{a} and R_{L}¹, R_{L}^{a} and R_{L}², R_{L}^{b} and R_{L}¹, or R_{L}^{b} and R_{L}² together with the atom(s) to which they are attached optionally form a C₃-C₁₂ carbocyclyl or 3-12 membered heterocyclyl;
each R_{L}^{e} and R_{L}^{f} is independently selected from the group consisting of F, OH, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl);
each R_{L}^{d} and R_{L}^{g} is independently selected from the group consisting of F, OH, C₁-C₄ alkyl, C₁-C₄ fluoroalkyl, C₃-C₆ cycloalkyl, C₁-C₄ alkoxy, C₃-C₆ cycloalkoxy, oxo, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); and
each R_{L}^{e} and R_{L}^{h} is independently selected from the group consisting of halo, OH, C₁-C₄ alkyl, C₁-C₄ fluoroalkyl, C₃-C₆ cycloalkyl, C₁-C₄ alkoxy, C₃-C₆ cycloalkoxy, oxo, CN, NH₂, NH(C₁-C₄ alkyl), N(C₁-C₄ alkyl)₂, NH(C₃-C₆ cycloalkyl), and N(C₁-C₄ alkyl)(C₃-C₆ cycloalkyl); or
two R_{L}^{c}, R_{L}^{d}, R_{L}^{c}, R_{L}^{f}, R_{L}^{g}, and R_{L}^{h} together with the atom(s) to which they are attached optionally form a C₃-C₁₂ carbocyclyl or 3-12 membered heterocyclyl.

18. The heterobifunctional compound or salt of claim 17, wherein L¹ comprises one or more R_{L}^{r} moieties selected from the group consisting of Formula (L-2), Formula (L-3), Formula (L-4), Formula (L-5) and Formula (L-6): 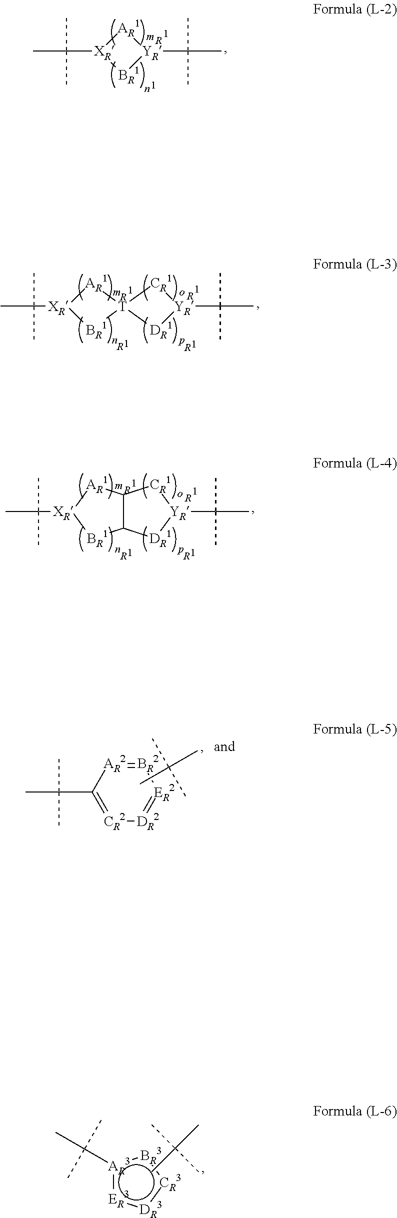
wherein:
X_{R}′ and Y_{R}′ are independently selected from N and CR_{R}^{b};
A_{R}¹, B_{R}¹, C_{R}¹ and D_{R}¹, at each occurrence, are independently selected from a bond, O, CO, SO, SO₂, C(O)NR_{R}^{b}, S(O)₂NR_{R}^{b}, NR_{R}^{b} and CR_{R}^{b}R_{R}^{c};
A_{R}², B_{R}², C_{R}², D_{R}², and E_{R}², at each occurrence, are independently selected from N and CR_{R}^{b};
A_{R}³, at each occurrence, is independently selected from N and C, and B_{R}³, C_{R}³, D_{R}³, and E_{R}³, at each occurrence, are independently selected from N, O, S, NR_{R}^{b} and CR_{R}^{b};
R_{R}^{b} and R_{R}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-12 membered carbocyclyl, optionally substituted 3-12 membered cycloalkoxy, optionally substituted 3-12 membered carbocyclylamino, optionally substituted 4-12 membered heterocyclyl, optionally substituted C₆-C₁₂ aryl, and optionally substituted 5-13 membered heteroaryl; or
two R_{R}^{b} and R_{R}^{c} together with the atom(s) to which they are attached optionally form a C₃-C₁₂ carbocyclyl or 3-12 membered heterocyclyl; and
m_{R}¹, n_{R}¹, o_{R}¹ and p_{R}¹ are independently selected from 0, 1, 2, 3, 4 and 5.

19. The heterobifunctional compound or salt of claim 17 or 18, wherein R_{L}^{r} is selected from the group consisting of:

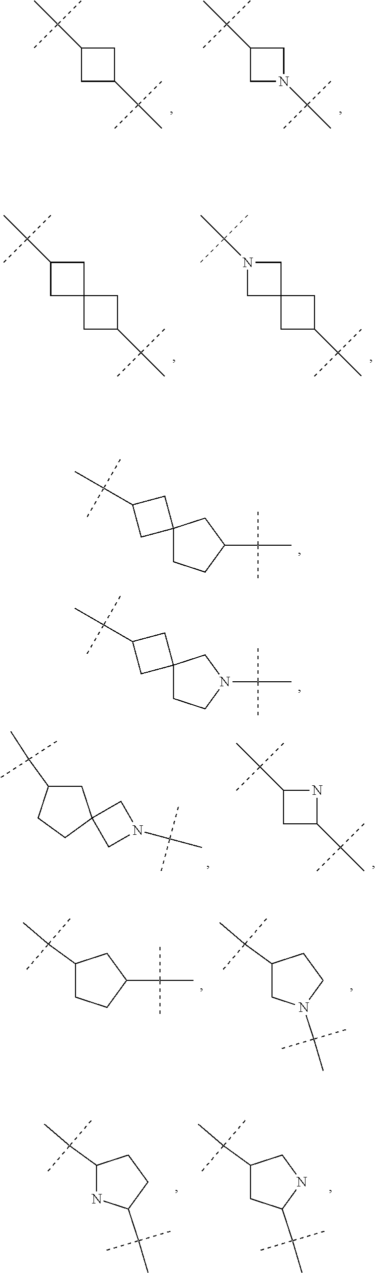

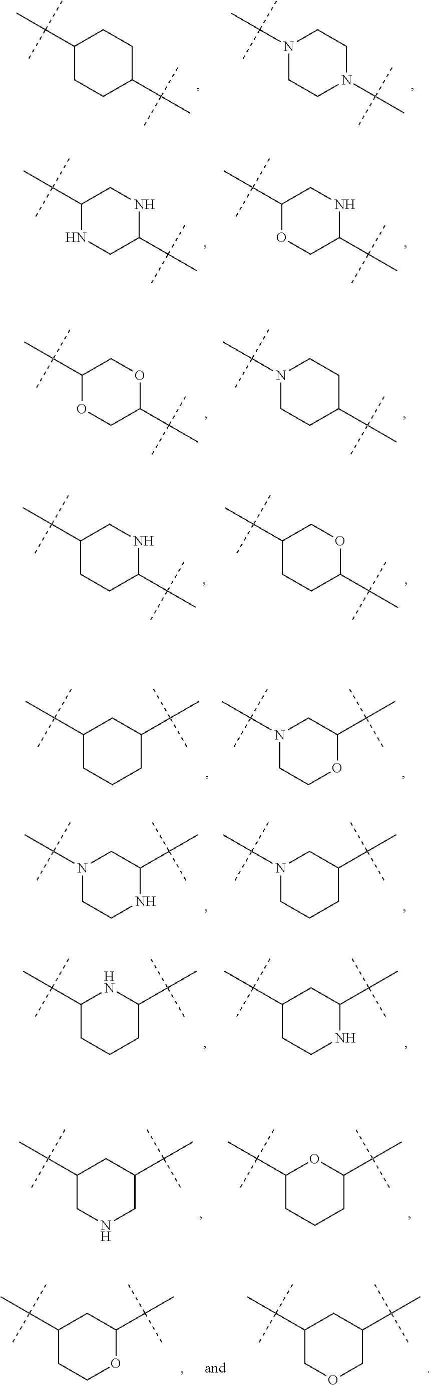

20. A pharmaceutical composition comprising the compound of any one of claims 1 to 19, or a pharmaceutically acceptable salt thereof, and at least one pharmaceutically acceptable excipient.

21. A compound of Formula (XIII): 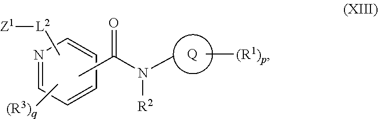
or a salt thereof, wherein:
Z¹ is L¹-Z² or L¹-G;
L¹ is a bond or a bivalent linker;
Z² is H, D, halo, C₁-C₄ alkyl, C₃-C₁₀ cycloalkyl, or 4-10 membered heterocyclyl, where each said C₁-C₄ alkyl, C₃-C₁₀ cycloalkyl, or 4-10 membered heterocyclyl is optionally substituted by halo, OH, oxo, C₁-C₄ alkoxy, NH₂, NH(C₁-C₄-alkyl), N(C₁-C₄-alkyl)₂;
G is a reactive functional group;
L² is a bond, —NH—, —N(C₁-C₃ alkyl)-, —NHC(O)—, —N(C₁-C₃ alkyl)C(O)—, —C(O)NH—, —C(O)N(C₁-C₃ alkyl)-, —O—, —C₁-C₄-alkylene-, —C₂-C₄-alkenylene- or —C₂-C₄-alkynylene-;
Q is C₆-C₁₀ aryl, 5-10 membered heteroaryl, C₃-C₁₀ cycloalkyl or 4-10 membered heterocyclyl;
each R¹ is independently R^{1A}, R^{1B}, R^{1C}, R^{1D} or R^{1E};
each R^{1A} is independently H, C₁-C₆ alkyl, C₃-C₆ cycloalkyl, or C₁-C₆ heteroalkyl, where each said C₁-C₆ alkyl, C₃-C₆ cycloalkyl, or C₁-C₆ heteroalkyl is optionally substituted by one or more R^{4A};
each R^{1B}, R^{1C}, R^{1D} and R^{1E} is independently H, C₁-C₆ alkyl, C₁-C₆ heteroalkyl, C₁-C₆ alkoxy, OH, D, halo, CN, NO₂, NR^{5A}R^{5B}, C(═O)R^{5A}, C(═O)OR^{5A}, OC(═O)R^{5A}, C(O)NR^{5A}R^{5B}, N(R^{5A})C(O)R^{5B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl, or C₁-C₆ alkoxy is optionally substituted by one or more R^{4B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{4C};
R² is H or C₁-C₃ alkyl;
each R³ is independently R^{3A}, R^{3B} or R^{3C}; or two R³ may be taken together with the atoms to which they are attached to form a C₃-C₁₀ cycloalkyl, 4-10 membered heterocyclyl, C₆-C₁₀ aryl or 5-10 membered heteroaryl, where each said C₃-C₁₆ cycloalkyl, 4-10 membered heterocyclyl, C₆-C₁₀ aryl or 5-10 membered heteroaryl is optionally substituted by one or more R^{6A};
each R^{3A}, R^{3B} and R^{3C} is independently C₁-C₆ alkyl, C₁-C₆ heteroalkyl, C₁-C₆ alkoxy, OH, D, halo, CN, NO₂, NR^{7A}R^{7B}, C(═O)R^{7A}, C(═O)OR^{7A}, OC(═O)R^{7A}, C(O)NR^{7A}R^{7B}, N(R^{7A})C(O)R^{7B}, C₃-C₁₀ cycloalkyl, C₃-C₁₀ cycloalkoxy, 4-10 membered heterocyclyl, C₆-C₁₀ aryl or 5-10 membered heteroaryl, where each said C₁-C₆ alkyl, C₁-C₆ heteroalkyl or C₁-C₆ alkoxy is optionally substituted by one or more R^{6B}, and each said C₃-C₁₀ cycloalkyl, C₃-C₁₀ cycloalkoxy, 4-10 membered heterocyclyl, C₆-C₁₀ aryl or 5-10 membered heteroaryl is optionally substituted by one or more R^{6C};
each R^{4A}, R^{4B} and R^{6B} is independently C₁-C₆ alkoxy, oxo, OH, D, halo, CN or NR^{8A}R^{8B};
each R^{4C}, R^{6A} and R^{6C} is independently C₁-C₄ alkyl, C₁-C₆ alkoxy, oxo, OH, D, halo, CN or NR^{9A}R^{9B};
each R^{5A}, R^{5B}, R^{7A}, R^{7B}, R^{8A}, R^{8B}, R^{9A} and R^{9B} is independently H, C₁-C₆ alkyl, C₁-C₆ heteroalkyl, C₃-C₁₀ cycloalkyl, 4-10 membered heterocyclyl, C₆-C₁₀ aryl or 5-10 membered heteroaryl, where each said C₁-C₆ alkyl or C₁-C₆ heteroalkyl is optionally substituted by one or more R^{10A}, and each said C₃-C₁₀ cycloalkyl, 4-10 membered heterocyclyl, C₆-C₁₀ aryl or 5-10 membered heteroaryl is optionally substituted by one or more R^{10B}; or
R^{5A} and R^{5B}, R^{7A} and R^{7B}, R^{8A} and R^{8B}, or R^{9A} and R^{9B} may be taken together with the N-atom to which they are attached to form a 4-10 membered heterocyclyl ring or 5-10 membered heteroaryl ring, where each said 4-10 membered heterocyclyl or 5-10 membered heteroaryl is optionally substituted by one or more R^{10C};
each R^{10A} is independently C₁-C₆ alkoxy, oxo, OH, D, halo, CN or NR^{11A}R^{11B};
each R^{10B} and R^{10C} is independently C₁-C₄ alkyl, C₁-C₆ alkoxy, oxo, OH, D, halo, CN or NR^{12A}R^{12B};
each R^{11A}, R^{11B}, R^{12A} and R^{12B} is independently H, C₁-C₄ alkyl, or C₃-C₆ cycloalkyl;
p is an integer from 0 to 4; and
q is an integer from 0 to 3;
with the proviso the compound of Formula (XIII) is not:
2-amino-N-(4,5-dimethylthiazol-2-yl)-6-methylnicotinamide;
tert-butyl (2-(2-(3-((3-((4,5-dimethylthiazol-2-yl)carbamoyl)-6-methylpyridin-2-yl)amino)-3-oxopropoxy)ethoxy)ethyl)carbamate;
2-(3-(2-(2-aminoethoxy)ethoxy)propanamido)-N-(4,5-dimethylthiazol-2-yl)-6-methylnicotinamide;
tert-butyl (7-((6-chloro-3-((4,5-dimethylthiazol-2-yl)carbamoyl)-pyridin-2-yl)amino)heptyl)carbamate;
tert-butyl (7-((3-((4,5-dimethylthiazol-2-yl)carbamoyl)-6-methylpyridin-2-yl)amino)heptyl)carbamate; or
2-((7-aminoheptyl)amino)-N-(4,5-dimethylthiazol-2-yl)-6-methylnicotinamide.

22. The compound of claim 21, having the structure of Formula (XIII-A), (XIII-B), (XIII-C) or (XIII-D): 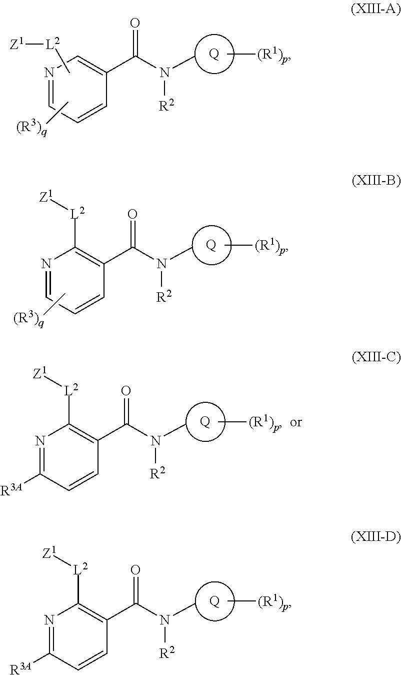
or a salt thereof.

23. The compound or salt of claim 21 or 22, wherein Q is selected from: 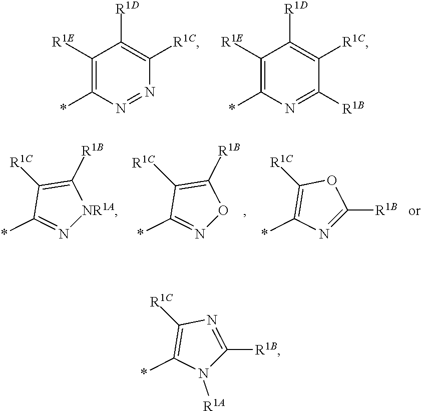
wherein * represents the point of attachment.

24. The compound or salt of any one of claims 21 to 23, Z¹ is L¹-G.

25. The compound or salt of any one of claims 21 to 24, wherein G is selected from the group consisting of protected or unprotected primary or secondary amine, carboxylic acid, carboxylate ester, halo, hydroxy, C₁-C₄ alkoxy, C₁-C₄ haloalkoxy, formyl, C₁-C₄ alkyl sulfonate ester, C₁-C₄ haloalkyl sulfonate ester, C₆-C₁₀ aryl sulfonate ester, boronic acid, boronate ester, and azide.

26. The compound or salt of any one of claims 21 to 23, wherein Z¹ is L¹-Z².

27. The compound or salt of any one of claims 21 to 26, wherein L¹ is a bond.

28. The compound or salt of any one of claims 21 to 26, wherein L¹ is a bivalent linker of Formula (L).

29. The compound or salt of any one of claims 21 to 26, wherein L¹ is a bivalent linker of Formula (L-1).

30. The compound or salt of any one of claims 21 to 26, wherein L¹ is a bivalent linker of Formula (J).

31. A method for the treatment of cancer in a subject in need thereof, comprising administering to the subject a therapeutically effective amount of a heterobifunctional compound of any one of claims 1 to 19, or a pharmaceutically acceptable salt thereof.

32. A method to degrade one or more proteins selected from CCND, CDK4 or CDK6, comprising administering a heterobifunctional compound of any one of claims 1 to 19, or a pharmaceutically acceptable salt thereof.

33. A method of degrading a protein selected from CCND, CDK4 and/or CDK6, comprising contacting the protein with a heterobifunctional compound of any one of claims 1 to 19, or a pharmaceutically acceptable salt thereof, wherein the contacting comprises contacting a cell comprising the protein with the heterobifunctional compound and results in degradation of the target protein.

34. A method of identifying a compound capable of degrading a protein selected from CCND, CDK4 and/or CDK6, comprising the steps of: (1) providing a test system for monitoring the degradation of the protein; and (2) determining whether a given test compound leads to degradation of the protein in said test system.


SEQUENCE LISTING (7 sequences)

SEQ ID NO: 1 - protein, 14 aa, Synthetic construct
ILPKVLHKRTLGLS

SEQ ID NO: 2 - protein, 14 aa, Synthetic construct
ILPKVWHKRELGLS

SEQ ID NO: 3 - protein, 13 aa, Synthetic construct
ILPKVLHKRTLGL

SEQ ID NO: 4 - protein, 13 aa, Synthetic construct
ILPKVLHKRTFGL

SEQ ID NO: 5 - protein, 9 aa, Synthetic construct
KVLHKRTLG

SEQ ID NO: 6 - protein, 13 aa, Synthetic construct
NFTSRLNRRASFP

SEQ ID NO: 7 - protein, 9 aa, Synthetic construct
SRLNRRASF